(19) United States
(12) Patent Application Publication
(10) Pub. No.: US 2024/0025906 A1
(43) Pub. Date: Jan. 25, 2024

(54) KINASE MODULATORS AND METHODS OF USE THEREOF

(71) Applicant: NEURON23, INC., South San Francisco, CA (US)

(72) Inventors: Zhonghua Pei, South San Francisco, CA (US); Gyanendra Kumar, South San Francisco, CA (US); Anh Truong, South San Francisco, CA (US); Claudio Aquino, South San Francisco, CA (US); Derek Kejia Wu, South San Francisco, CA (US); Andrew Tasker, South San Francisco, CA (US)

(21) Appl. No.: 18/210,766

(22) Filed: Jun. 16, 2023

Related U.S. Application Data
(60) Provisional application No. 63/353,104, filed on Jun. 17, 2022.

(51) Int. Cl. C07D 487/04

(52) U.S. Cl. CPC C07D 487/04

(57) ABSTRACT

The invention provides compounds that modulate the activity of protein kinases that are associated with human diseases, disorders, and conditions. In particular, compounds of the invention inhibit TYK2, a member of the Janus Kinase (JAK) family of non-receptor protein kinases.

### FIELD OF THE INVENTION

The invention provides compounds that modulate the activity of kinases, such as Tyrosine Kinase 2 (TYK2).

### BACKGROUND

A variety of medical conditions that affect millions of people are caused or exacerbated by unregulated activity of protein kinases. For example, aberrant kinase activity is associated with autoimmune diseases, inflammatory diseases, bone diseases, metabolic diseases, neurological and neurodegenerative diseases, cancer, cardiovascular diseases, allergies, asthma, Alzheimer's disease, Parkinson's disease, skin disorders, eye diseases, infectious diseases and hormone-related diseases. For many such disorders, however, no effective inhibitor or activator exists for the particular kinase that causes the disorder or its symptoms. Consequently, patients continue to suffer from an array of disorders due to the lack of suitable medicaments for their conditions.

### SUMMARY

Janus tyrosine kinase (JAK) family members are regulators of multiple signal transduction pathways initiated by membrane Type 1 and Type II cytokine receptors. There are 4 JAK family members including JAK1, JAK2, JAK3, and TYK2 (Schwartz et al. 2017). One such association is with signal transducer and activator of transcription (STAT) signal transduction mediated cytokine responses. The JAK-STAT signaling pathway is a chain of interactions between proteins in a cell, and is involved in processes such as immunity, cell division, cell death, and tumor formation (Aaronson et al Science 2002). The binding of Type I and Type II cytokine receptor ligands, such as interferons and interleukins, to cell-surface receptors, causes the receptors to dimerize, which brings the receptor-associated JAKs into close proximity (Jalini et al. Genes and Cancer 2011), and sets off a sequence of downstream changes. There is a large body of evidence establishing the contribution of JAK-dependent cytokines to immunopathology, and clinical benefit can be provided by blocking these cytokines with biologics and small-molecule inhibitors. Some examples of this are the blockade of IL-6 in rheumatoid arthritis or IL-12/IL-23 in inflammatory bowel disease (IBD) (Schwartz et al 2017).

The tyrosine kinase 2 (TYK2) member of the JAK family specifically plays a role in the downstream signaling of Interleukin (IL)-12, IL-23, and type I interferons (Baker and Isaacs, Ann Rheum Dis., 2018; Burke et al, Sci Trans Med. 2019). Like other JAK family members, TYK2 heterodimerizes with other JAK family members to provide ligand specificity and regulate downstream signal transduction pathways (FIG. 1). Many of these pathways are altered in diseases and drive chronic inflammation in IBD, Psoriasis, and systemic lupus erythematosus (SLE) (Schwartz et al, Nat Rev Drug Dis, 2017).

In addition to the role of TYK2 signaling cascades in disease there has been a strong body of genetic evidence of pointing to a role for TYK2. Genetic association studies have linked the TYK2 locus to an impact of the susceptibility in SLE, psoriasis, and multiple sclerosis (MS). This identification has been replicated and expanded in a number of recent analyses, and TYK2 is now recognized as a susceptibility gene in a variety of inflammatory and autoimmune diseases, including type I diabetes (TID). The common characteristic of these diseases are changes in immunological function and activation, and downstream damage to target organs (Li et al, PLOS One, 2020).

The use of small-molecule inhibitors of TYK2 have allowed for the confirmation of several of these hypotheses. Previous work in human derived PBMCs have demonstrated the ability of TYK2 inhibition to reduce IL-12/IL-23 signaling in rodents and humans TYK2 inhibition has also proven efficacious in preclinical models of disease for psoriasis and ulcerative colitis (Burke et al, Sci Trans Med, 2020). The preclinical effects in rodents have since translated to humans with deucravacitinib demonstrating efficacy in Psoriasis patients (Armstrong et al, Ann of Rheu Dis, 2020). The genetic contribution of TYK2 has also been confirmed preclinically with the use of TYK2 knockout (KO) or transgenic (TG) animals. For example, Type I interferon signaling is reduced in in TYK2 KO animals as compared to WT mice (Karaghiosoff, Immunity, 2000) and TG animals with the P1104 protective variant of TYK2 are almost completely protected in the experimental autoimmune encephalitis (EAE) mouse model of MS (Gorman et al, Frnt in Immunology, 2019). Together, this large body of evidence provides supportive data for the role of cytokine signaling, and the support for the development of safe TYK2 inhibitors for a variety of inflammatory disorders.

The invention provides compounds that modulate the activity of protein kinases that are associated with human diseases, disorders, and conditions. In particular, compounds of the invention inhibit TYK2, a member of the Janus Kinase (JAK) family of non-receptor protein kinases. Altered or unregulated activity of TYK2 promotes inflammation and is implicated in autoimmune diseases, such as psoriasis, lupus, multiple sclerosis, and inflammatory bowel disease. Thus, embodiments of the invention are useful as pharmaceutical compositions for treatment of such autoimmune conditions. The invention also provides methods of using the compounds to modulate kinase activity in cells and to treat conditions, such as autoimmune conditions, for which modulation of kinase activity provides a therapeutic benefit.

In an aspect, the compound of the invention is a compound of formula (I):

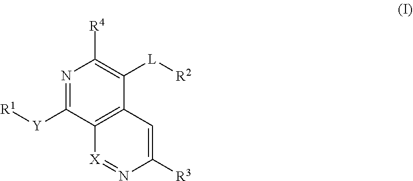

and pharmaceutically acceptable salts thereof,
wherein:
X is CH or N;
Y is CH₂, S, or NH;
L is a single bond, double bond, triple bond substituted or unsubstituted alkyl, heteroalkyl, alkoxy, heteroalkoxy, cycloalkyl, heterocycloalkyl, haloalkyl, halocycloalkyl, aryl, heteroaryl, —C(O)NH—, —NHC(O)—, O, NH, or S;
R¹ is alkyl, cycloalkyl, haloalkyl, halocycloalkyl, aryl, heteroaryl, methyl, CD₃, or H;
R² is halo, alkyl, branched alkyl, alkenyl, alkynyl, haloalkyl, alkoxy, cycloalkoxy, haloalkoxy, nitro, cyano, or a 5 or 6 membered substituted or unsubstituted aryl, or monocyclic or bicyclic heteroaryl ring optionally containing one or more heteroatoms independently selected from O, S, and N,
wherein the substitutions on the said 5 or 6 membered aryl or heteroaryl rings are: H, halo, alkyl, branched alkyl, alkenyl, alkynyl, haloalkyl, alkoxy, cycloalkoxy, haloalkoxy, thioalkyl, nitro, cyano, —CH₂-cycloalkyl, —CF₂-cycloalkyl, —CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —C(═O)NH₂, hydroxy, —COOH (and ester thereof), sulfonyl, alkylsulfonyl, arylsulfonyl, sulfonamide, amino, 3-6 membered cycloalkyl or heterocycloalkyl, 3-6 membered aryl or heteroaryl, any of which may have one or more substituents;

R³ is H, halo, alkyl, branched alkyl, alkenyl, alkynyl, cycloalkyl, haloalkyl, alkoxy, cycloalkoxy, haloalkoxy, nitro, cyano, aminoalkyl, aminocycloalkyl, aminoheterocycloalkyl, —NH-aryl, —NH-heteroaryl, —NH-phenyl, —NH₂, —NH—CH—CF₃, substituted or unsubstituted C(═O)cycloalkyl, substituted or unsubstituted —NH—C(═O)cycloalkyl, —NH—C(═O)alkyl, substituted or unsubstituted —NH—C(═O)cycloalkyl, substituted or unsubstituted aminoalkylaryl; and R⁴ is selected from a group consisting of: H, halo, alkyl, branched alkyl, alkenyl, alkynyl, cycloalkyl, haloalkyl, alkoxy, cycloalkoxy, haloalkoxy, nitro, cyano, and alkylhydroxyl.

In certain embodiments, Y is NH.

In certain embodiments, R₁ is methyl or ethyl.

In certain embodiments, R₁, is a single bond.

In certain embodiments, X is CH.

In certain embodiments, X is N.

In certain embodiments, R³ is:

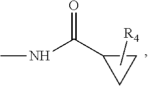

wherein R⁴ is H, halo, alkyl, branched alkyl, alkenyl, alkynyl, cycloalkyl, spirocycloalkyl haloalkyl, alkoxy, cycloalkoxy, haloalkoxy, nitro, or cyano.

In certain embodiments, R³ is:

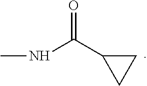

In certain embodiments, R² is:

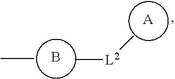

wherein L² is substituted or unsubstituted alkyl; heteroalkyl, alkoxy, heteroalkoxy, cycloalkyl, heterocycloalkyl, haloalkyl, halocycloalkyl, aryl, heteroaryl, or a bond linking the groups;
A or B are independently 5 or 6 membered substituted or unsubstituted aryl or heteroaryl ring optionally containing one or more heteroatoms independently selected from O, S, and N, wherein the substitutions on the said 5 or 6 membered aryl or heteroaryl ring are: H, halo; alkyl, branched alkyl, alkenyl, alkynyl, haloalkyl, alkoxy, cycloalkoxy, haloalkoxy, nitro, cyano, —CH₂-cycloalkyl, —CF₂-cycloalkyl, —CH(CH₃)-cycloalkyl, —CH₇-aryl, —CF₂-aryl, —CH(—CH)-aryl, C(═O)-alkyl, —C(═O)cycloalkyl, —C(═O)—NH-alkyl, —C(═O)NH₂, hydroxy, —COOH (and ester thereof), alkylsulfonyl, arylsulfonyl, sulfonamide, amino, 3-6 membered cycloalkyl or heterocycloalkyl, 3-6 membered aryl or heteroaryl, any of which may have one or more substituents.

In certain embodiments, R² is phenyl.

In certain embodiments, R² is:

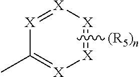

wherein each X is independently N or CH;
R⁵ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NH(C₂O)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O) R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, and substituted or unsubstituted heteroaryl, and n is 0, 1, 2, or 3.

In certain embodiments, R² is:

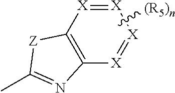

wherein Z is O or S, each X is independently N or CH;
R₅ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NHR⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, substituted or unsubstituted heteroaryl; and n is 0, 1, 2 or 3.

In certain embodiments, R² is:

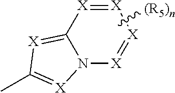

wherein each X is independently N or CH; R⁵ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroarkyl, substituted or unsubstituted spirobicycloheteroalkyl, substituted or unsubstituted aryl, substituted or unsubstituted heteroaryl, —NHR⁶, NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, substituted or unsubstituted heteroaryl; and n is 0, 1, 2 or 3.

In certain embodiments, L-R² is:

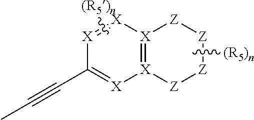

wherein each X is independently N or CH;
Z is independently O or NR⁶

R⁵ and R^{5′} is independently H, halogen, hydroxyl, —CN, alkyl; haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —N—H(CO)R⁶, —C(O)R⁶, —C(O)NH R⁶, —S(O)R⁶; S(O)NHR⁶, —S(O)(NH)R⁶, —S(O)(NMe)R⁶, —(CH₂)ₙS(O)R⁶, —(CH₂)ₙOR⁶, —P(O) R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted-bridgedbicycloheteroalkyl, substituted or unsubstituted spirobicycloheteroalkyl, substituted or unsubstituted aryl, or substituted or unsubstituted heteroaryl; and n=1, 2 or 3.

In certain embodiments, L-R² is:

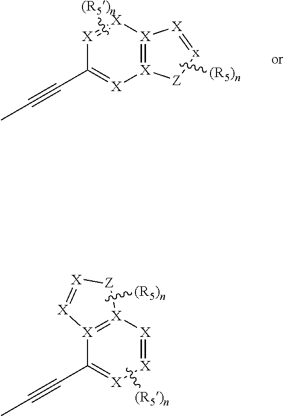

wherein Z is N or O,
each X is independently N or CH;
R⁵ and R^{5′} is independently H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted-bridgedbicycloheteroalkyl, substituted or unsubstituted spirobicycloheteroalkyl, substituted or unsubstituted aryl, or substituted or unsubstituted heteroaryl; and
n is 0, 1, 2, or 3.

In certain embodiments, L-R² is:

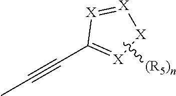

wherein R₅ is selected from a group consisting of: H, halogen, hydroxyl, —CN, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, S(O)NHR⁶, —S(O)(NH)R⁶, —S(O)(NMe)R⁶, —(CH₂)ₙS(O)R⁶, —P(O)R⁶R^{6′} where R⁶ and R^{6′} is independently alkyl, branched alkyl, haloalkyl, substituted or unsubstituted, cycloalkyl, substituted or un substituted cycloheteroalkyl, substituted or un substituted fusedbicycloheteroalkyl, substituted or unsubstituted bridgedbicycloheteroalkyl, substituted or unsubstituted spirobicycloheteroalkyl, substituted or unsubstituted aryl, and substituted or unsubstituted heteroaryl; and n is 0, 1, 2, or 3.

In another preferred embodiment, the compound of formula (I) is selected from the group consisting of:

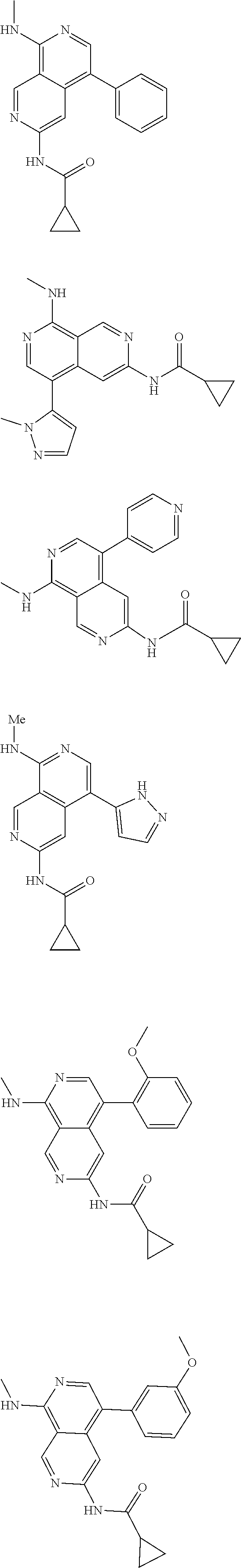

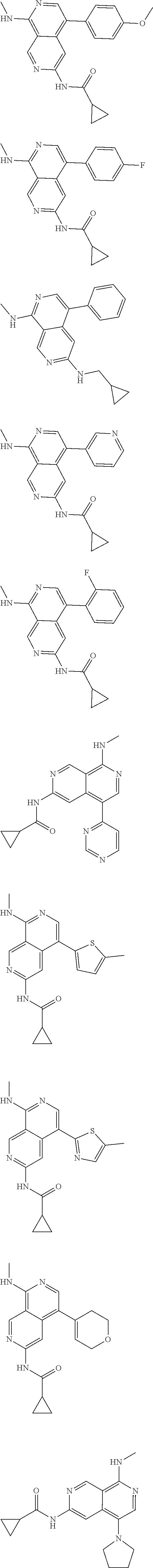

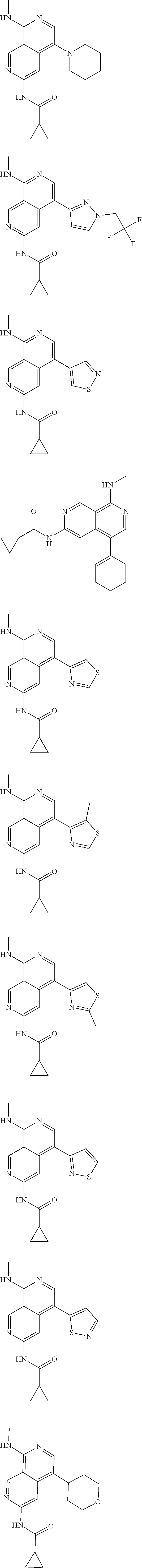

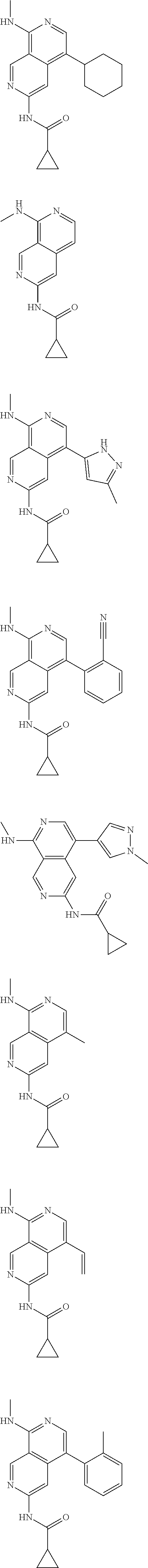

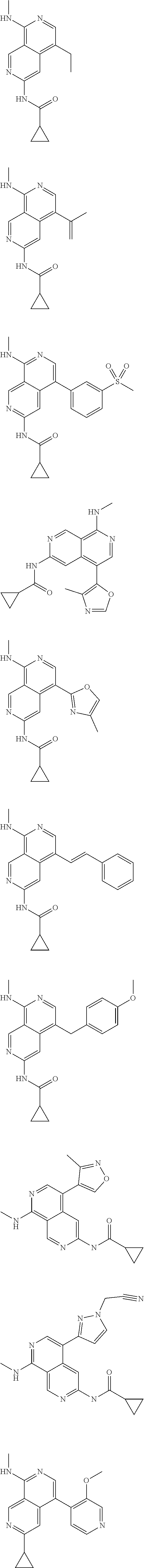

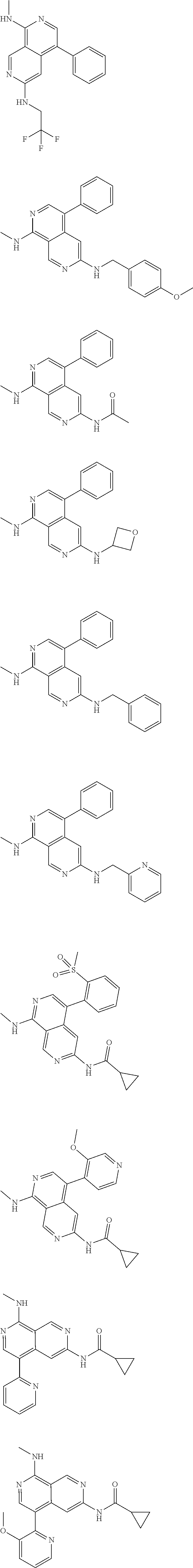

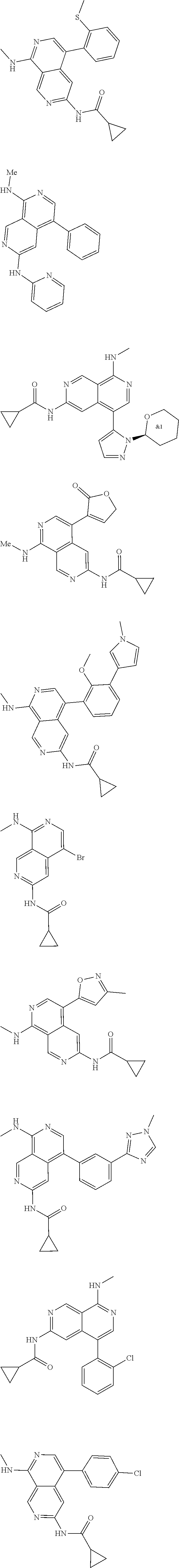

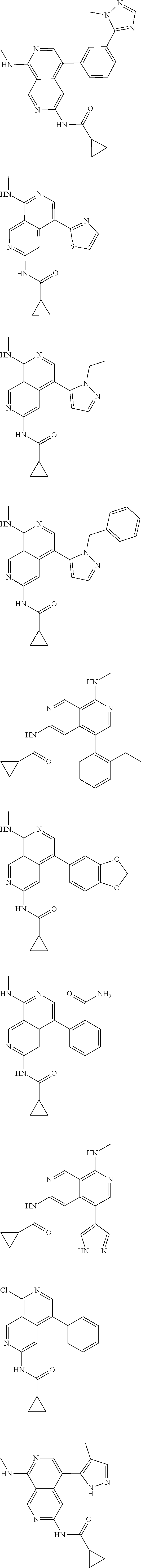

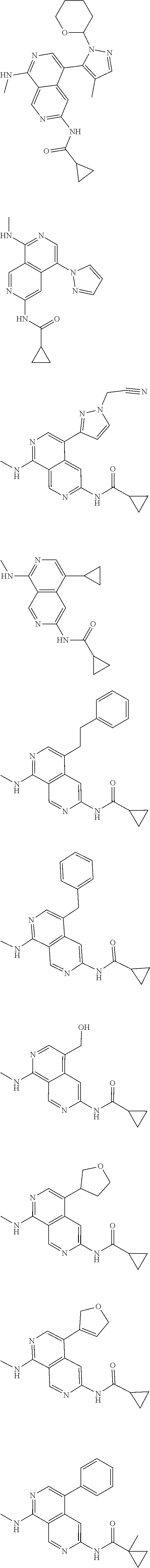

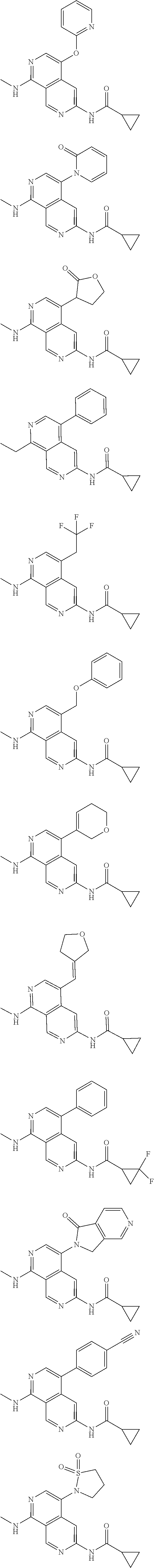

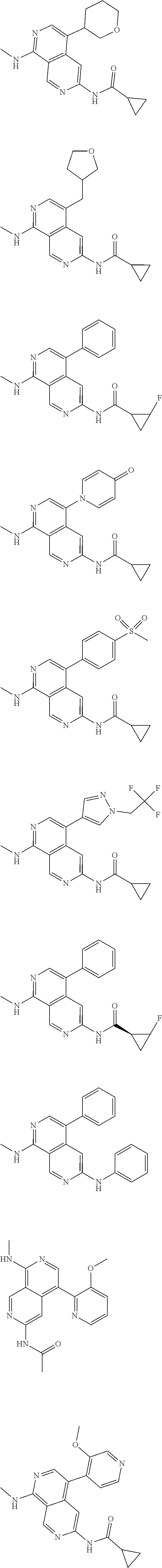

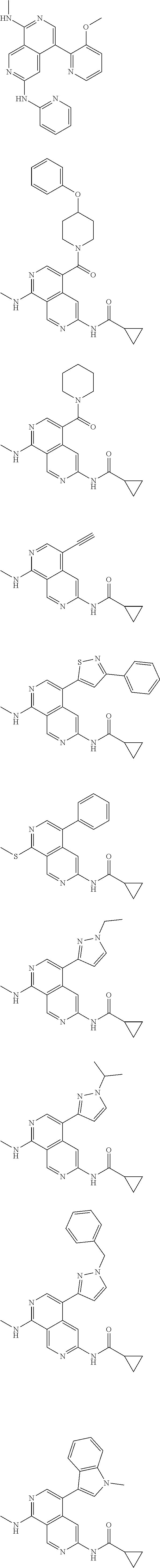

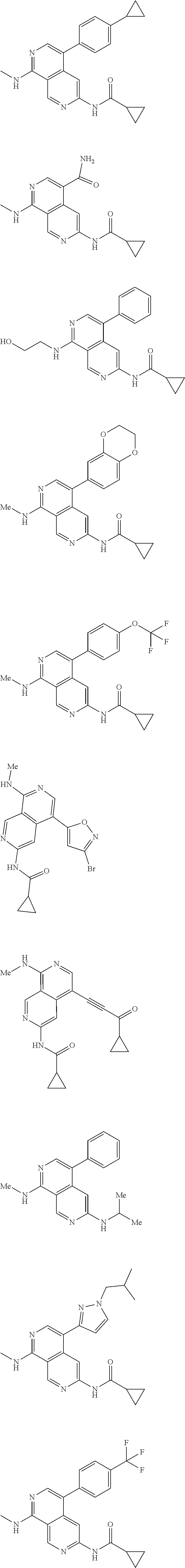

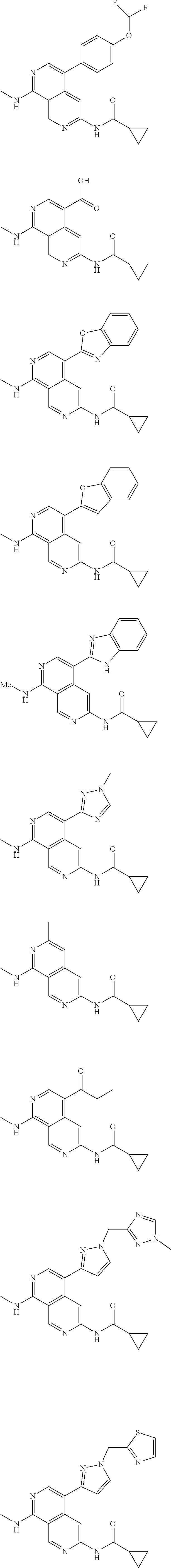

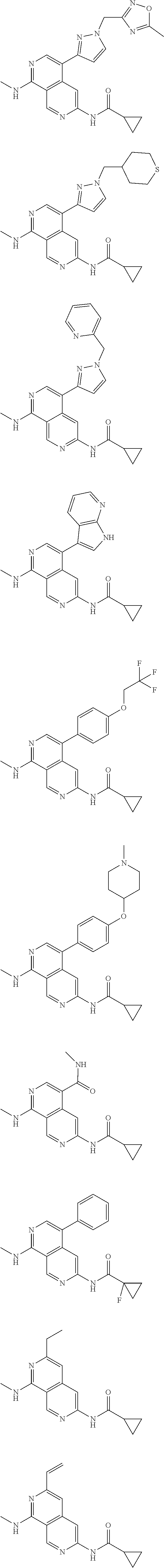

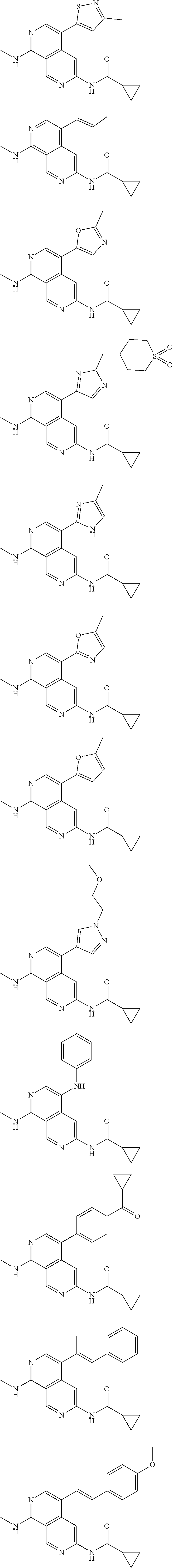

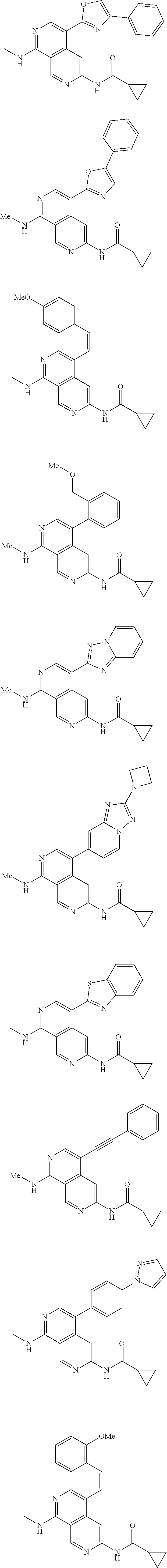

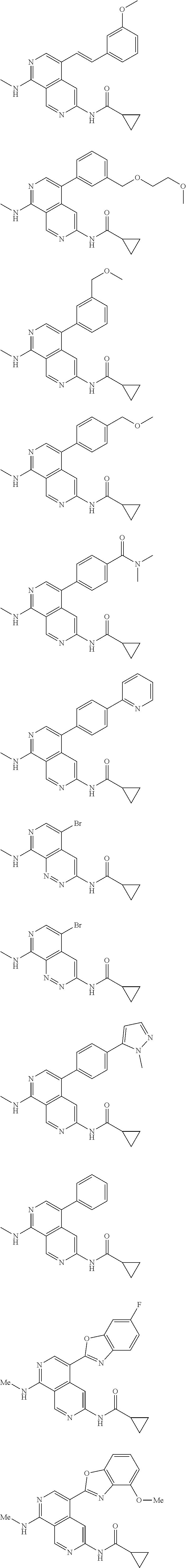

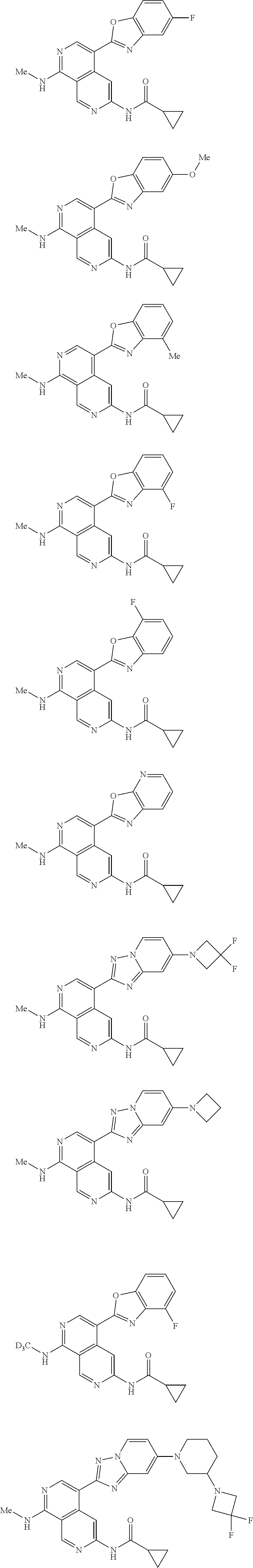

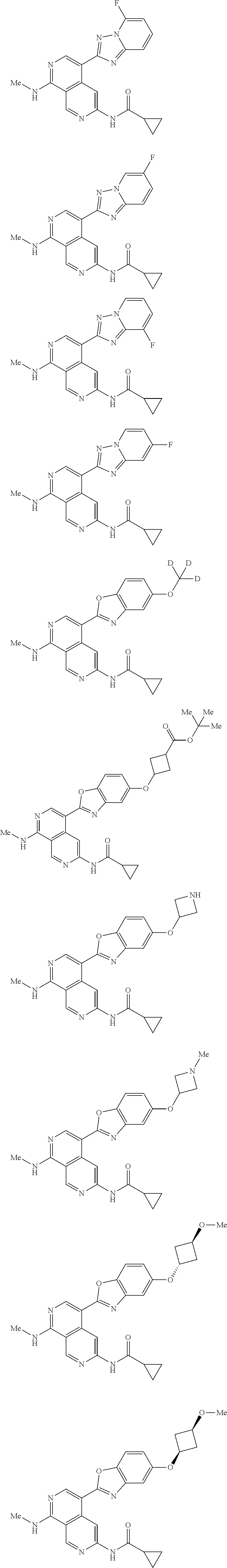

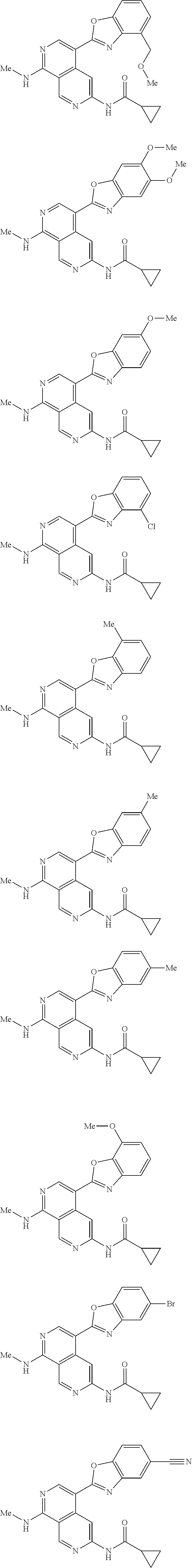

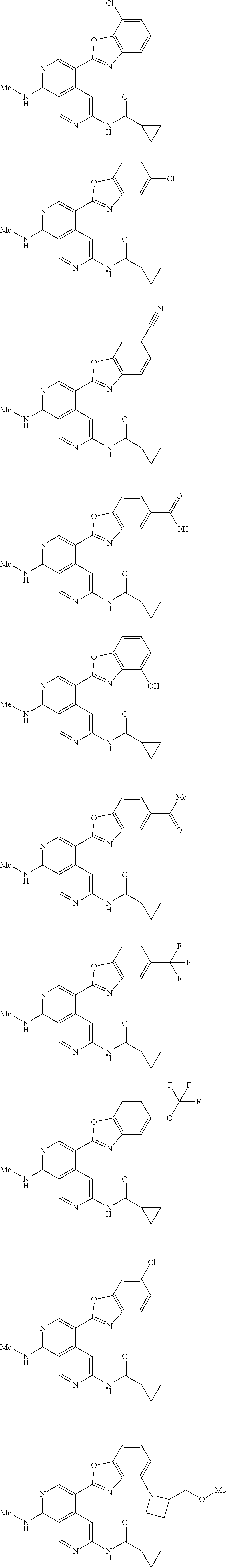

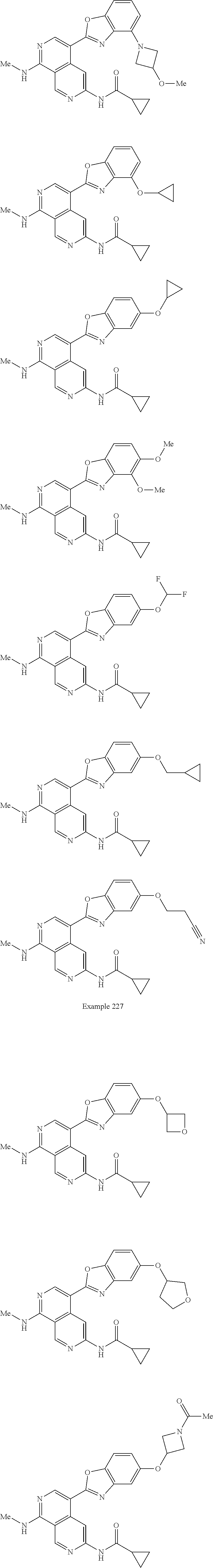

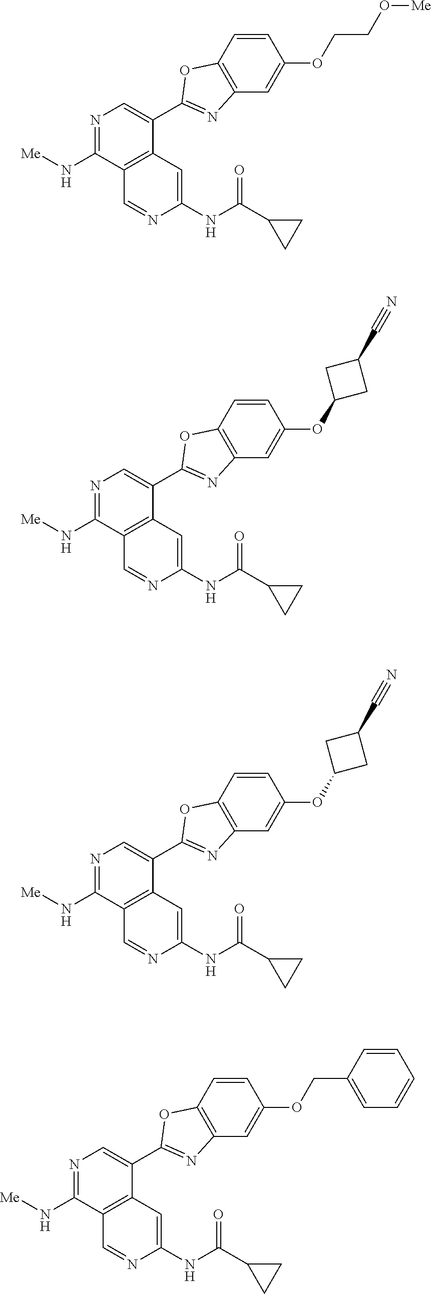 [SMILES: CNc1ncc(-c2nc3cc(OCCOC)ccc3o2)c2cc(NC(=O)C3CC3)ncc12 ; CNc1ncc(-c2nc3cc(O[C@H]4C[C@@H](C#N)C4)ccc3o2)c2cc(NC(=O)C3CC3)ncc12 ; CNc1ncc(-c2nc3cc(O[C@H]4C[C@H](C#N)C4)ccc3o2)c2cc(NC(=O)C3CC3)ncc12 ; CNc1ncc(-c2nc3cc(OCc4ccccc4)ccc3o2)c2cc(NC(=O)C3CC3)ncc12]

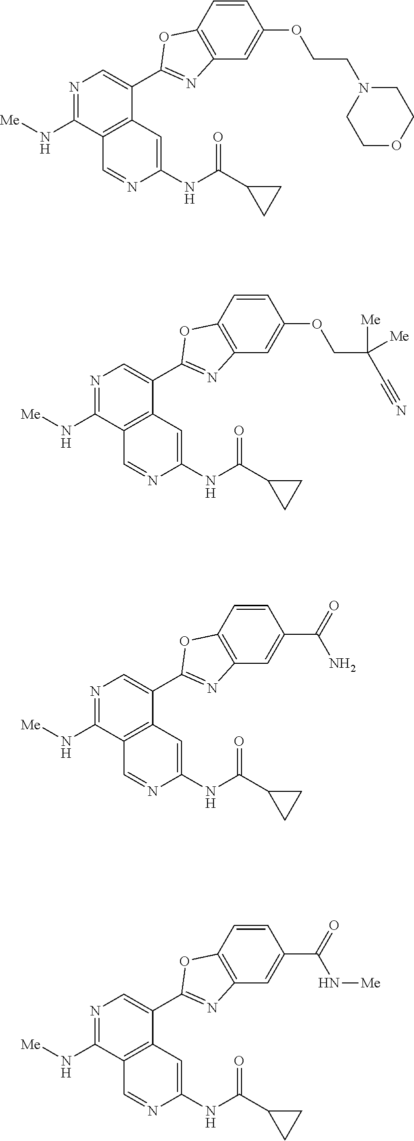 [SMILES: CNc1ncc(-c2nc3cc(OCCN4CCOCC4)ccc3o2)c2cc(NC(=O)C3CC3)ncc12 ; CNc1ncc(-c2nc3cc(OCC(C)(C)C#N)ccc3o2)c2cc(NC(=O)C3CC3)ncc12 ; CNc1ncc(-c2nc3cc(C(N)=O)ccc3o2)c2cc(NC(=O)C3CC3)ncc12 ; CNC(=O)c1ccc2oc(-c3cnc(NC)c4cnc(NC(=O)C5CC5)cc34)nc2c1]

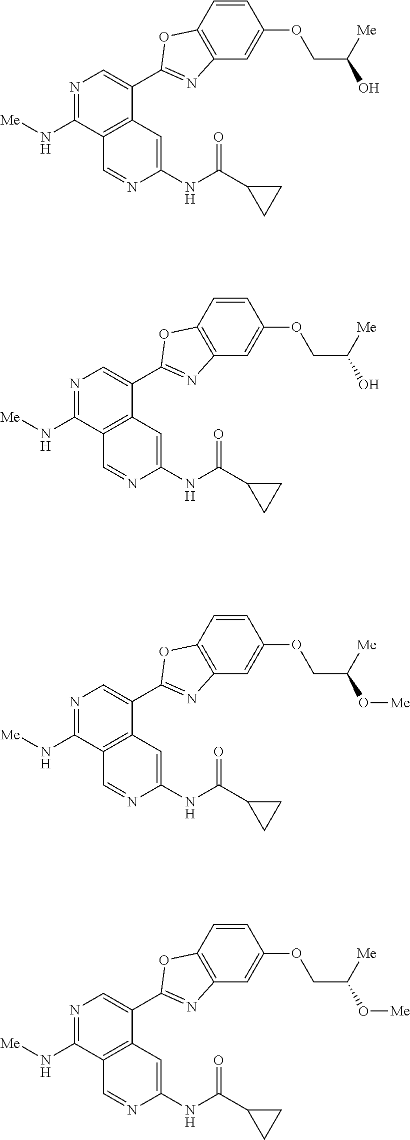 [SMILES: CNc1ncc(-c2nc3cc(OC[C@@H](C)O)ccc3o2)c2cc(NC(=O)C3CC3)ncc12 ; CNc1ncc(-c2nc3cc(OC[C@H](C)O)ccc3o2)c2cc(NC(=O)C3CC3)ncc12 ; CNc1ncc(-c2nc3cc(OC[C@@H](C)OC)ccc3o2)c2cc(NC(=O)C3CC3)ncc12 ; CNc1ncc(-c2nc3cc(OC[C@H](C)OC)ccc3o2)c2cc(NC(=O)C3CC3)ncc12]

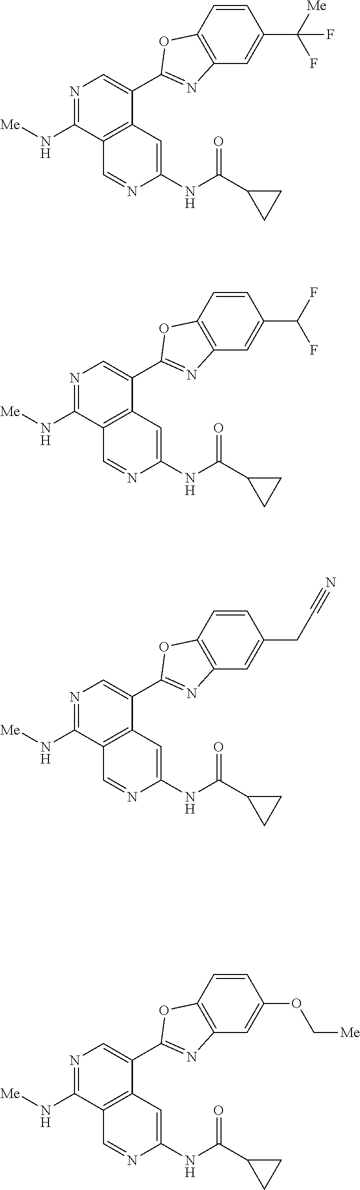 [SMILES: CNc1ncc(-c2nc3cc(C(C)(F)F)ccc3o2)c2cc(NC(=O)C3CC3)ncc12 ; CNc1ncc(-c2nc3cc(C(F)F)ccc3o2)c2cc(NC(=O)C3CC3)ncc12 ; CNc1ncc(-c2nc3cc(CC#N)ccc3o2)c2cc(NC(=O)C3CC3)ncc12 ; CCOc1ccc2oc(-c3cnc(NC)c4cnc(NC(=O)C5CC5)cc34)nc2c1]

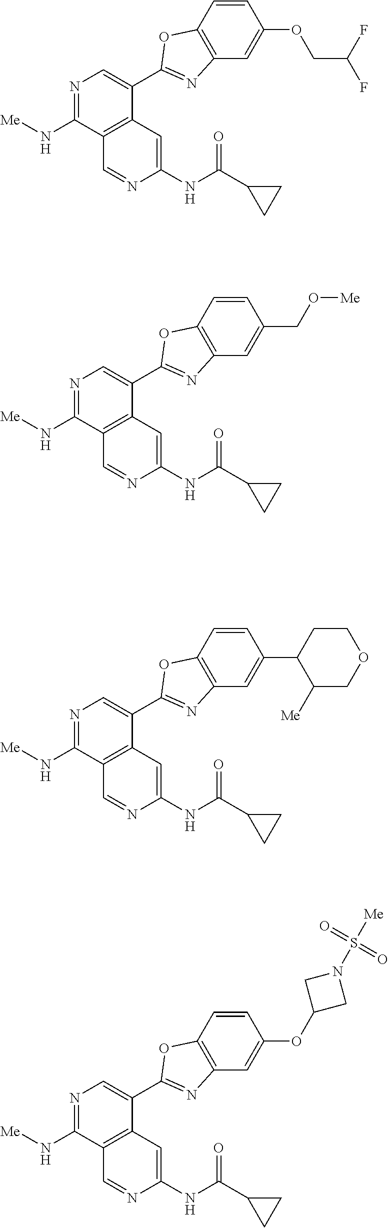 [SMILES: CNc1ncc(-c2nc3cc(OCC(F)F)ccc3o2)c2cc(NC(=O)C3CC3)ncc12 ; CNc1ncc(-c2nc3cc(COC)ccc3o2)c2cc(NC(=O)C3CC3)ncc12 ; CNc1ncc(-c2nc3cc(C4CCOCC4C)ccc3o2)c2cc(NC(=O)C3CC3)ncc12 ; CNc1ncc(-c2nc3cc(OC4CN(S(C)(=O)=O)C4)ccc3o2)c2cc(NC(=O)C3CC3)ncc12]

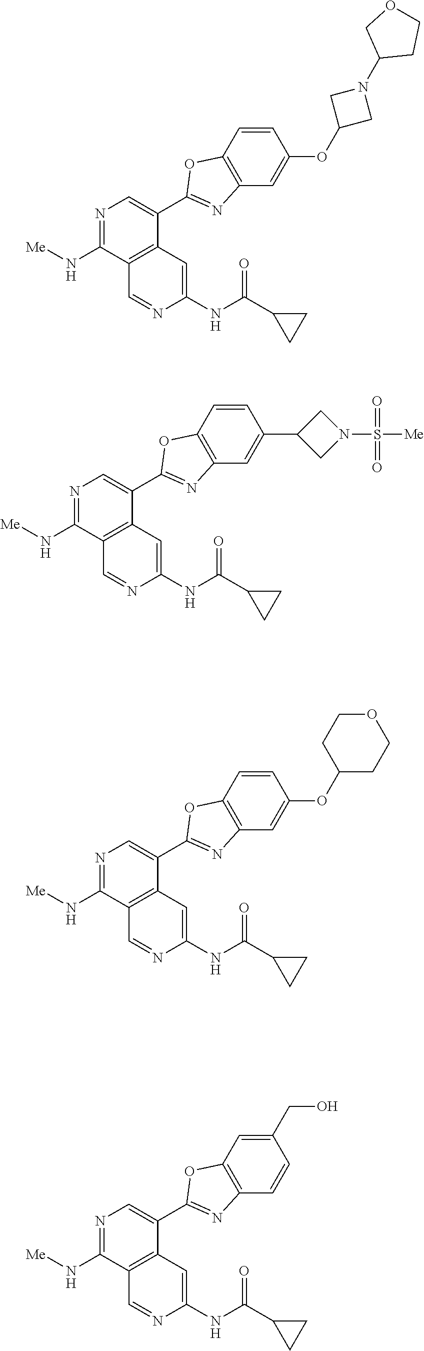 [SMILES: CNc1ncc(-c2nc3cc(OC4CN(C5CCOC5)C4)ccc3o2)c2cc(NC(=O)C3CC3)ncc12 ; CNc1ncc(-c2nc3cc(C4CN(S(C)(=O)=O)C4)ccc3o2)c2cc(NC(=O)C3CC3)ncc12 ; CNc1ncc(-c2nc3cc(OC4CCOCC4)ccc3o2)c2cc(NC(=O)C3CC3)ncc12 ; CNc1ncc(-c2nc3ccc(CO)cc3o2)c2cc(NC(=O)C3CC3)ncc12]

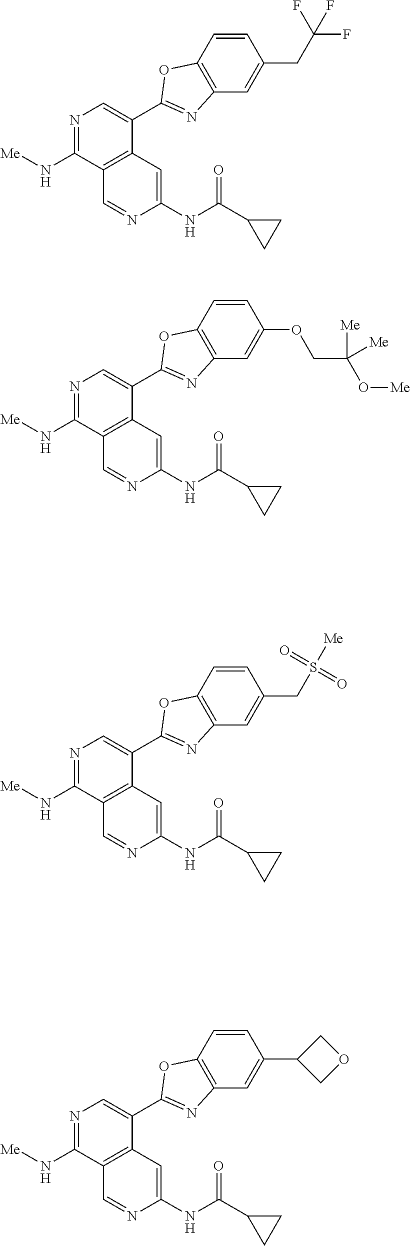 [SMILES: CNc1ncc(-c2nc3cc(CC(F)(F)F)ccc3o2)c2cc(NC(=O)C3CC3)ncc12 ; CNc1ncc(-c2nc3cc(OCC(C)(C)OC)ccc3o2)c2cc(NC(=O)C3CC3)ncc12 ; CNc1ncc(-c2nc3cc(CS(C)(=O)=O)ccc3o2)c2cc(NC(=O)C3CC3)ncc12 ; CNc1ncc(-c2nc3cc(C4COC4)ccc3o2)c2cc(NC(=O)C3CC3)ncc12]

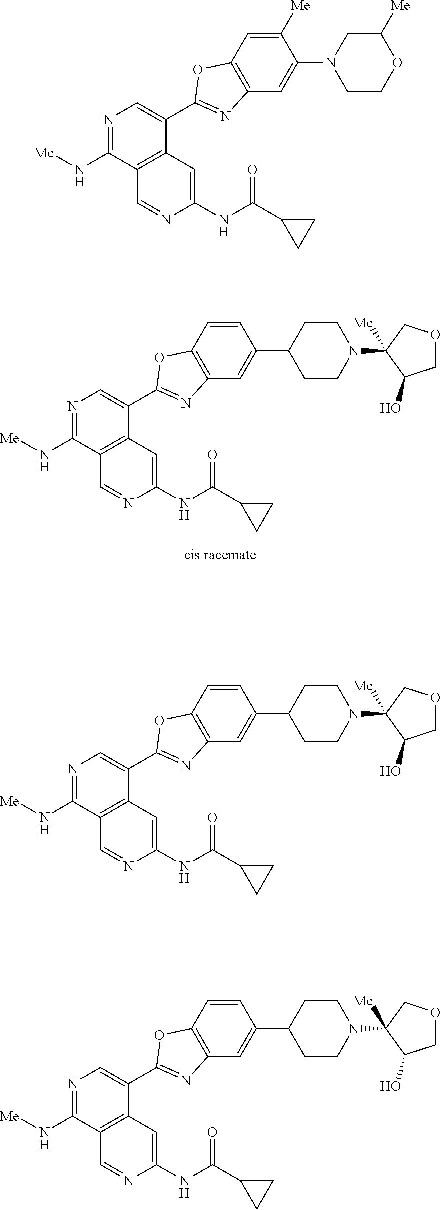 [SMILES: CNc1ncc(-c2nc3cc(N4CCOC(C)C4)c(C)cc3o2)c2cc(NC(=O)C3CC3)ncc12 ; CNc1ncc(-c2nc3cc(C4CCN([C@@]5(C)COC[C@H]5O)CC4)ccc3o2)c2cc(NC(=O)C3CC3)ncc12 ; CNc1ncc(-c2nc3cc(C4CCN([C@@]5(C)COC[C@H]5O)CC4)ccc3o2)c2cc(NC(=O)C3CC3)ncc12 ; CNc1ncc(-c2nc3cc(C4CCN([C@]5(C)COC[C@@H]5O)CC4)ccc3o2)c2cc(NC(=O)C3CC3)ncc12]

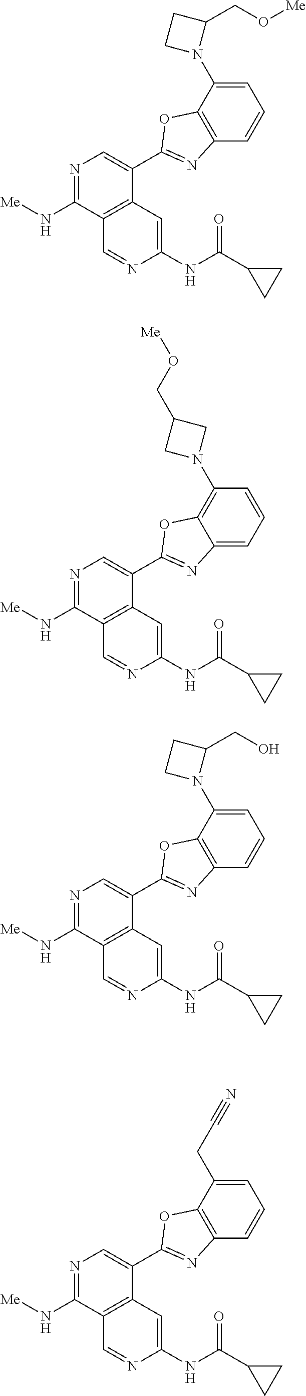

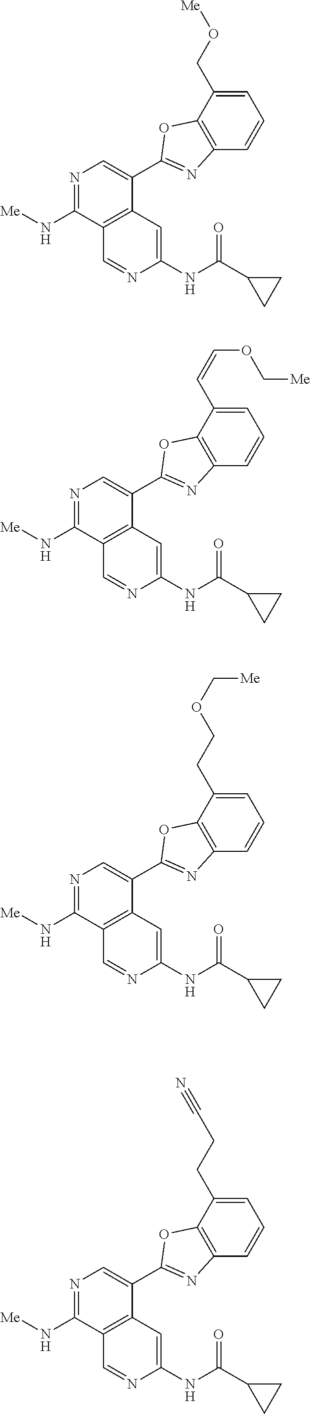 [SMILES: CNc1ncc(-c2nc3cccc(COC)c3o2)c2cc(NC(=O)C3CC3)ncc12 ; CCO/C=C\c1cccc2nc(-c3cnc(NC)c4cnc(NC(=O)C5CC5)cc34)oc12 ; CCOCCc1cccc2nc(-c3cnc(NC)c4cnc(NC(=O)C5CC5)cc34)oc12 ; CNc1ncc(-c2nc3cccc(CCC#N)c3o2)c2cc(NC(=O)C3CC3)ncc12]

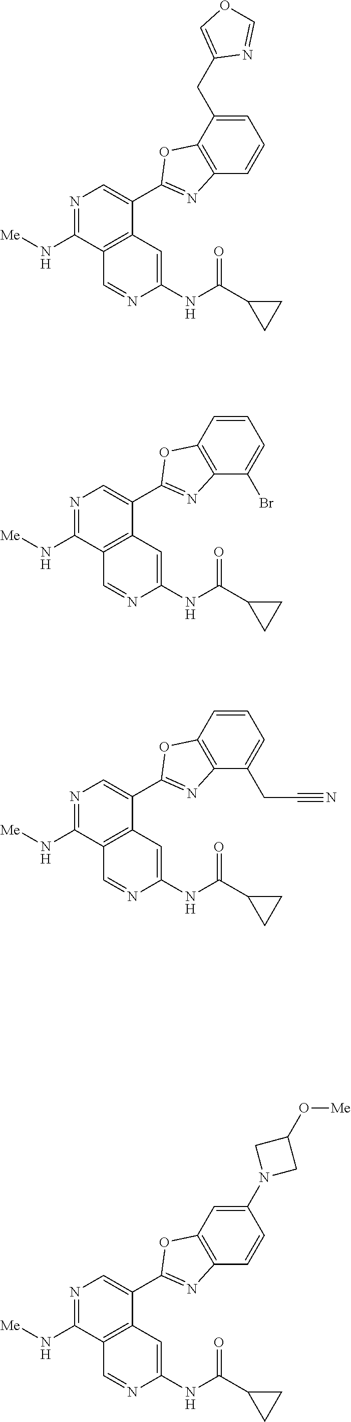 [SMILES: CNc1ncc(-c2nc3cccc(Cc4cocn4)c3o2)c2cc(NC(=O)C3CC3)ncc12 ; CNc1ncc(-c2nc3c(Br)cccc3o2)c2cc(NC(=O)C3CC3)ncc12 ; CNc1ncc(-c2nc3c(CC#N)cccc3o2)c2cc(NC(=O)C3CC3)ncc12 ; CNc1ncc(-c2nc3ccc(N4CC(OC)C4)cc3o2)c2cc(NC(=O)C3CC3)ncc12]

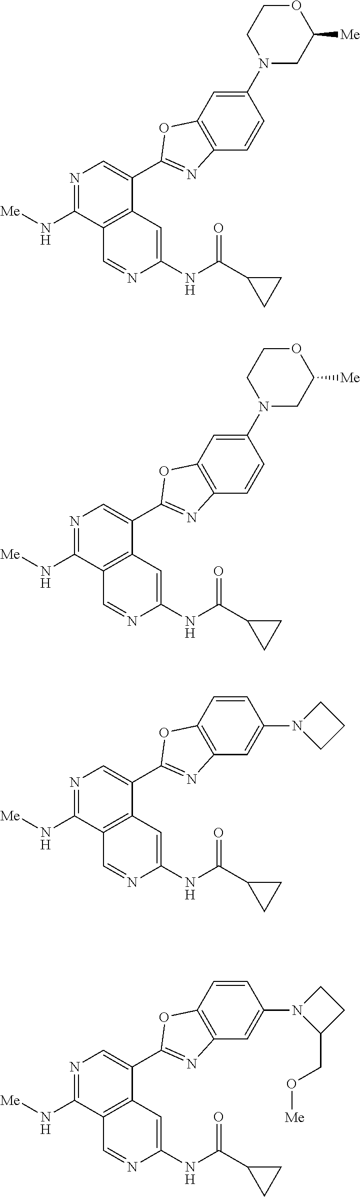

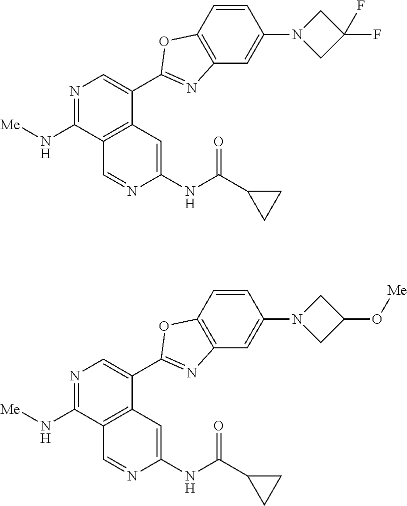 [SMILES: CNc1ncc(-c2nc3cc(N4CC(F)(F)C4)ccc3o2)c2cc(NC(=O)C3CC3)ncc12 ; CNc1ncc(-c2nc3cc(N4CC(OC)C4)ccc3o2)c2cc(NC(=O)C3CC3)ncc12]

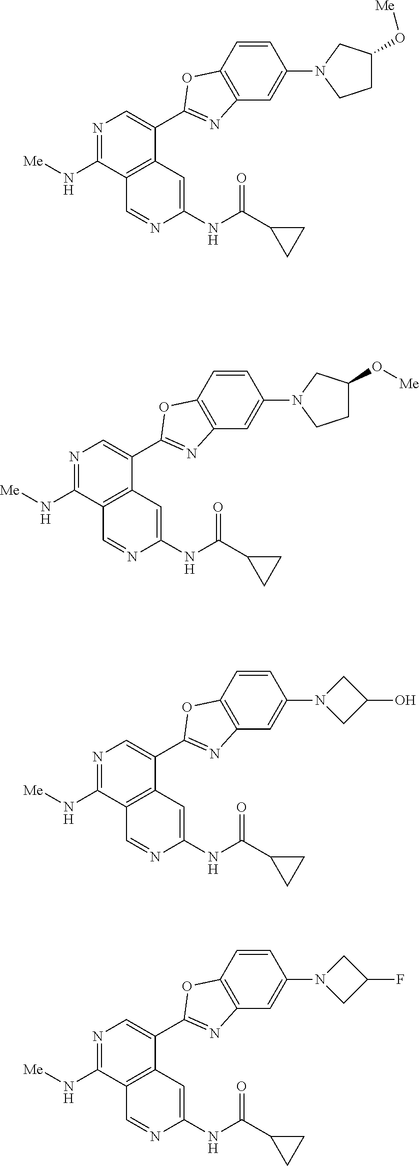 [SMILES: CNc1ncc(-c2nc3cc(N4CC[C@@H](OC)C4)ccc3o2)c2cc(NC(=O)C3CC3)ncc12 ; CNc1ncc(-c2nc3cc(N4CC[C@H](OC)C4)ccc3o2)c2cc(NC(=O)C3CC3)ncc12 ; CNc1ncc(-c2nc3cc(N4CC(O)C4)ccc3o2)c2cc(NC(=O)C3CC3)ncc12 ; CNc1ncc(-c2nc3cc(N4CC(F)C4)ccc3o2)c2cc(NC(=O)C3CC3)ncc12]

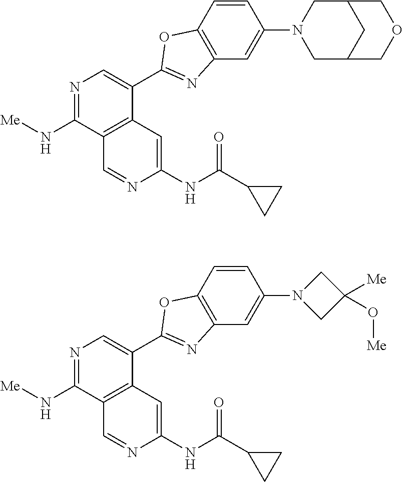 [SMILES: CNc1ncc(-c2nc3cc(N4CC5COCC(C5)C4)ccc3o2)c2cc(NC(=O)C3CC3)ncc12 ; CNc1ncc(-c2nc3cc(N4CC(C)(OC)C4)ccc3o2)c2cc(NC(=O)C3CC3)ncc12]

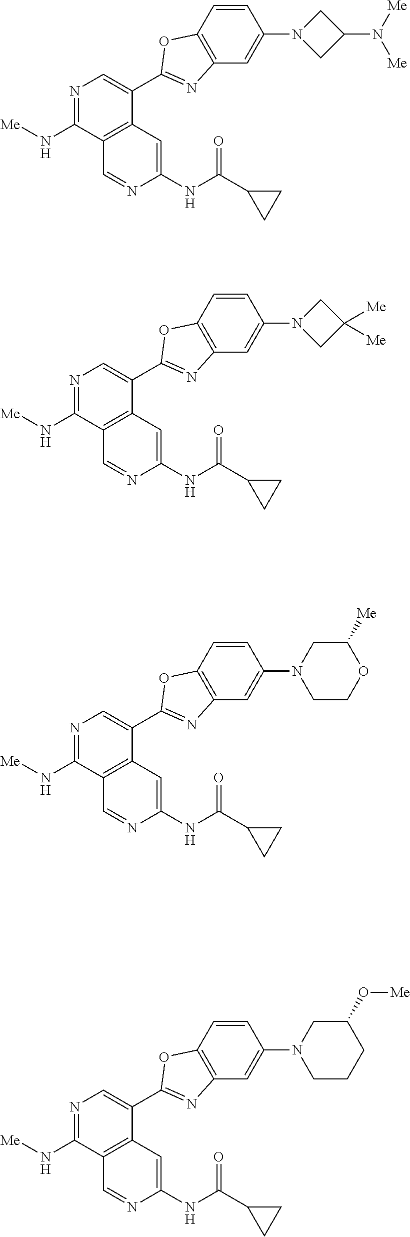 [SMILES: CNc1ncc(-c2nc3cc(N4CC(N(C)C)C4)ccc3o2)c2cc(NC(=O)C3CC3)ncc12 ; CNc1ncc(-c2nc3cc(N4CC(C)(C)C4)ccc3o2)c2cc(NC(=O)C3CC3)ncc12 ; CNc1ncc(-c2nc3cc(N4CCO[C@@H](C)C4)ccc3o2)c2cc(NC(=O)C3CC3)ncc12 ; CNc1ncc(-c2nc3cc(N4CCC[C@@H](OC)C4)ccc3o2)c2cc(NC(=O)C3CC3)ncc12]

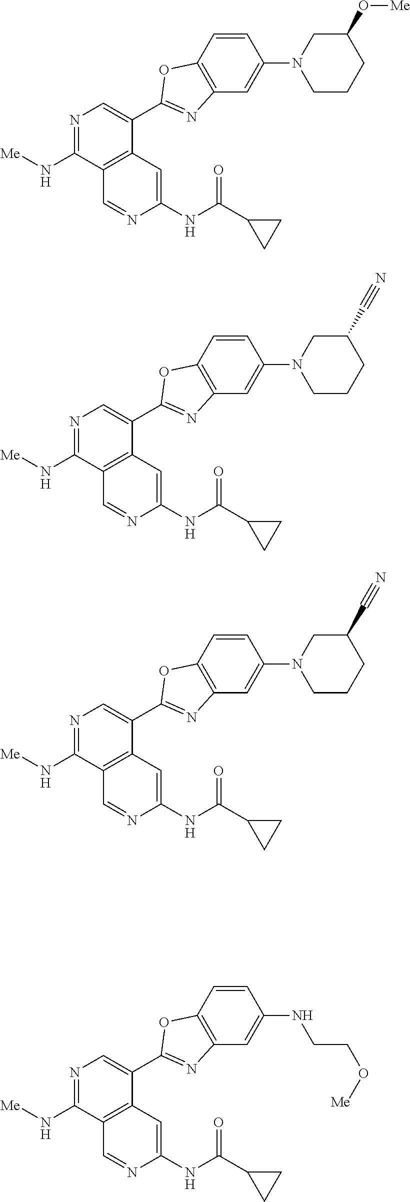 [SMILES: CNc1ncc(-c2nc3cc(N4CCC[C@H](OC)C4)ccc3o2)c2cc(NC(=O)C3CC3)ncc12 ; CNc1ncc(-c2nc3cc(N4CCC[C@@H](C#N)C4)ccc3o2)c2cc(NC(=O)C3CC3)ncc12 ; CNc1ncc(-c2nc3cc(N4CCC[C@H](C#N)C4)ccc3o2)c2cc(NC(=O)C3CC3)ncc12 ; CNc1ncc(-c2nc3cc(NCCOC)ccc3o2)c2cc(NC(=O)C3CC3)ncc12]

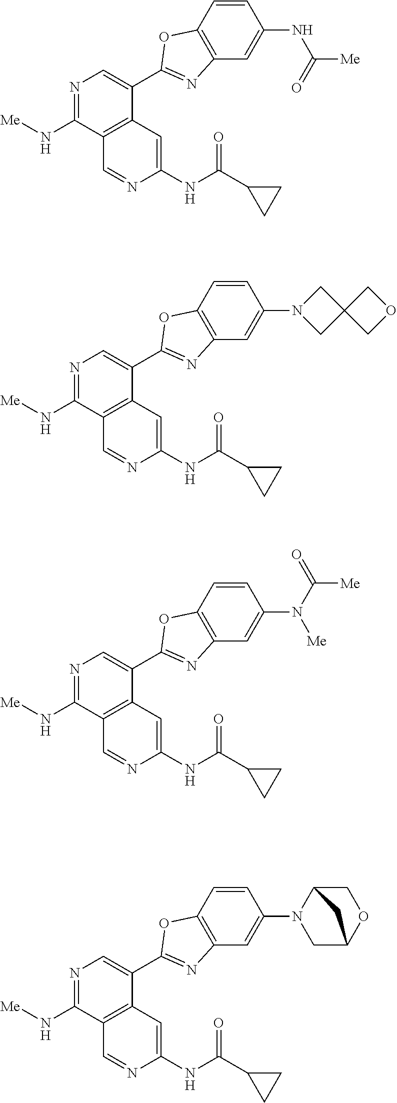 [SMILES: CNc1ncc(-c2nc3cc(NC(C)=O)ccc3o2)c2cc(NC(=O)C3CC3)ncc12 ; CNc1ncc(-c2nc3cc(N4CC5(COC5)C4)ccc3o2)c2cc(NC(=O)C3CC3)ncc12 ; CNc1ncc(-c2nc3cc(N(C)C(C)=O)ccc3o2)c2cc(NC(=O)C3CC3)ncc12 ; CNc1ncc(-c2nc3cc(N4C[C@@H]5C[C@H]4CO5)ccc3o2)c2cc(NC(=O)C3CC3)ncc12]

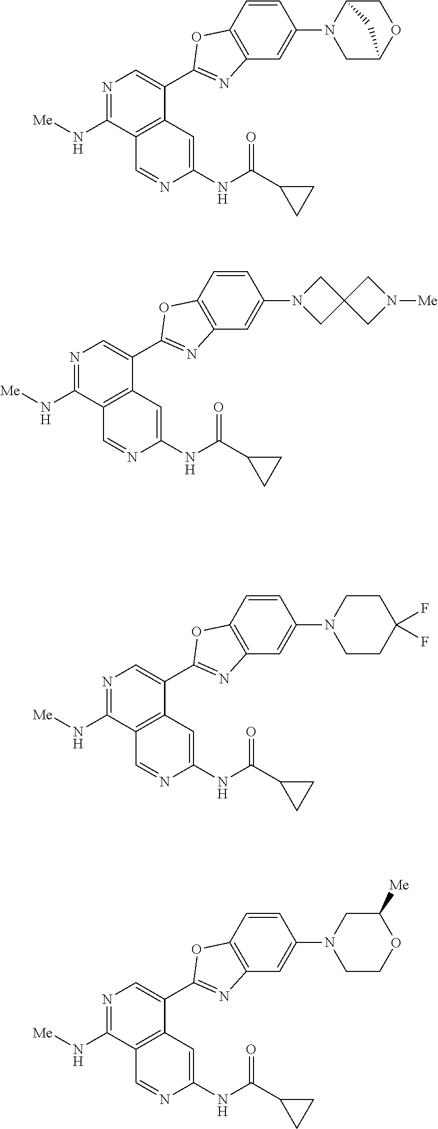 [SMILES: CNc1ncc(-c2nc3cc(N4C[C@H]5C[C@@H]4CO5)ccc3o2)c2cc(NC(=O)C3CC3)ncc12 ; CNc1ncc(-c2nc3cc(N4CC5(CN(C)C5)C4)ccc3o2)c2cc(NC(=O)C3CC3)ncc12 ; CNc1ncc(-c2nc3cc(N4CCC(F)(F)CC4)ccc3o2)c2cc(NC(=O)C3CC3)ncc12 ; CNc1ncc(-c2nc3cc(N4CCO[C@H](C)C4)ccc3o2)c2cc(NC(=O)C3CC3)ncc12]

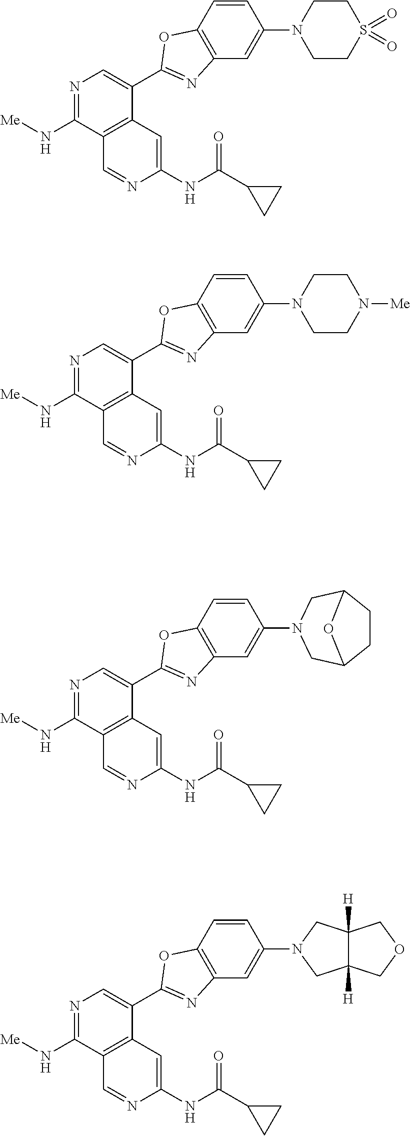 [SMILES: CNc1ncc(-c2nc3cc(N4CCS(=O)(=O)CC4)ccc3o2)c2cc(NC(=O)C3CC3)ncc12 ; CNc1ncc(-c2nc3cc(N4CCN(C)CC4)ccc3o2)c2cc(NC(=O)C3CC3)ncc12 ; CNc1ncc(-c2nc3cc(N4CC5CCC(C4)O5)ccc3o2)c2cc(NC(=O)C3CC3)ncc12 ; CNc1ncc(-c2nc3cc(N4C[C@H]5COC[C@H]5C4)ccc3o2)c2cc(NC(=O)C3CC3)ncc12]

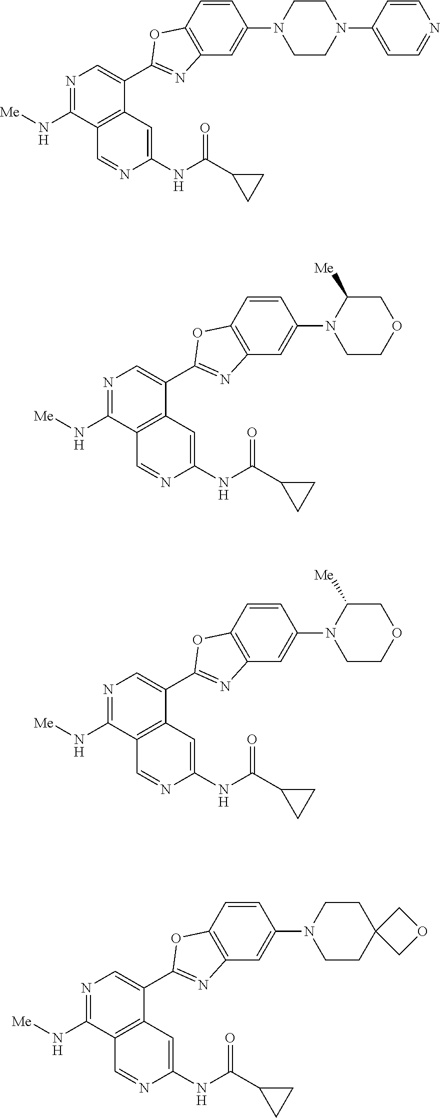 [SMILES: CNc1ncc(-c2nc3cc(N4CCN(c5ccncc5)CC4)ccc3o2)c2cc(NC(=O)C3CC3)ncc12 ; CNc1ncc(-c2nc3cc(N4CCOC[C@@H]4C)ccc3o2)c2cc(NC(=O)C3CC3)ncc12 ; CNc1ncc(-c2nc3cc(N4CCOC[C@H]4C)ccc3o2)c2cc(NC(=O)C3CC3)ncc12 ; CNc1ncc(-c2nc3cc(N4CCC5(CC4)COC5)ccc3o2)c2cc(NC(=O)C3CC3)ncc12]

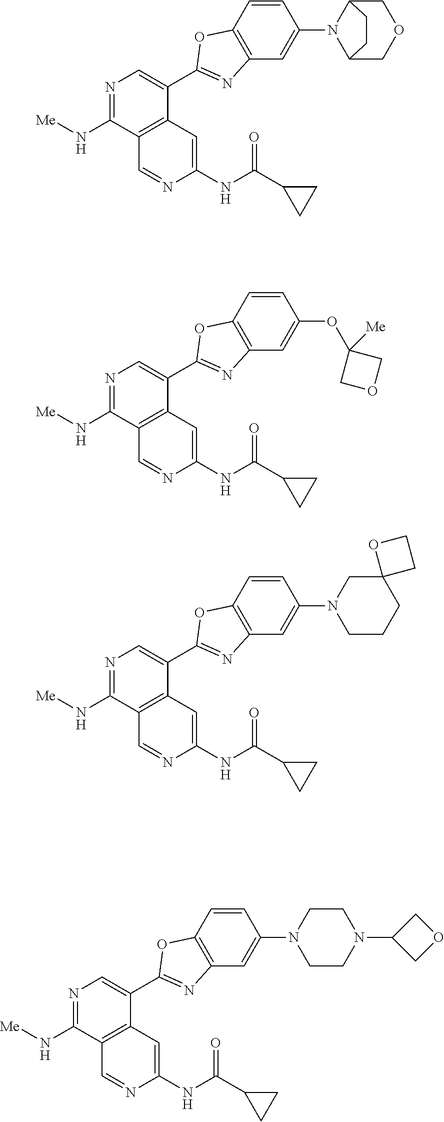 [SMILES: CNc1ncc(-c2nc3cc(N4C5CCC4COC5)ccc3o2)c2cc(NC(=O)C3CC3)ncc12 ; CNc1ncc(-c2nc3cc(OC4(C)COC4)ccc3o2)c2cc(NC(=O)C3CC3)ncc12 ; CNc1ncc(-c2nc3cc(N4CCCC5(CCO5)C4)ccc3o2)c2cc(NC(=O)C3CC3)ncc12 ; CNc1ncc(-c2nc3cc(N4CCN(C5COC5)CC4)ccc3o2)c2cc(NC(=O)C3CC3)ncc12]

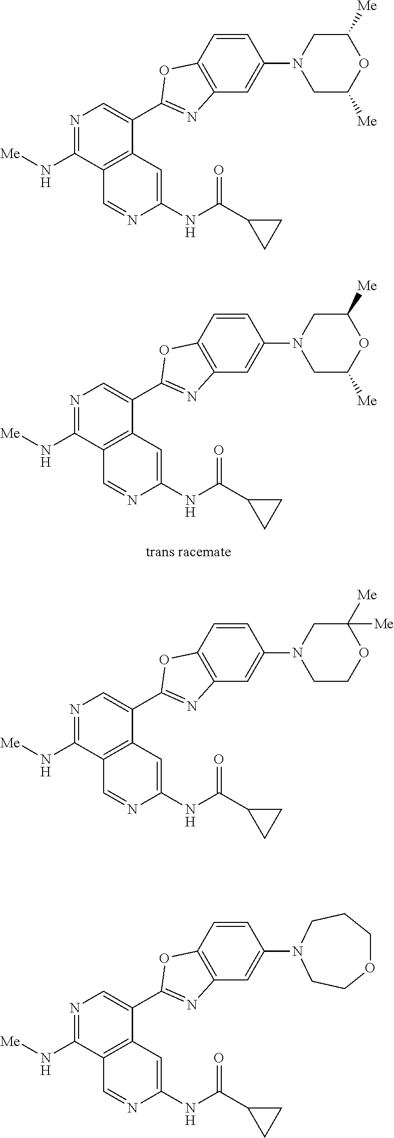 [SMILES: CNc1ncc(-c2nc3cc(N4C[C@@H](C)O[C@@H](C)C4)ccc3o2)c2cc(NC(=O)C3CC3)ncc12 ; CNc1ncc(-c2nc3cc(N4C[C@@H](C)O[C@H](C)C4)ccc3o2)c2cc(NC(=O)C3CC3)ncc12 ; CNc1ncc(-c2nc3cc(N4CCOC(C)(C)C4)ccc3o2)c2cc(NC(=O)C3CC3)ncc12 ; CNc1ncc(-c2nc3cc(N4CCCOCC4)ccc3o2)c2cc(NC(=O)C3CC3)ncc12]

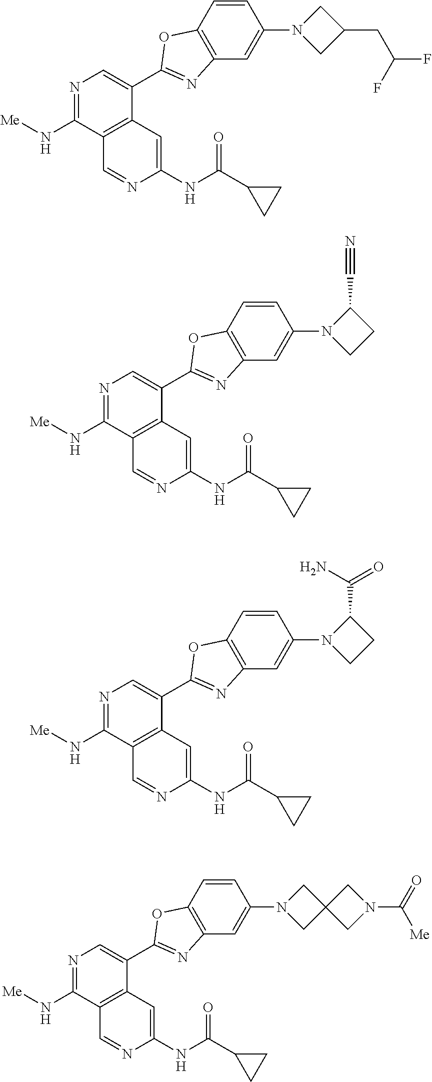 [SMILES: CNc1ncc(-c2nc3cc(N4CC(CC(F)F)C4)ccc3o2)c2cc(NC(=O)C3CC3)ncc12 ; CNc1ncc(-c2nc3cc(N4CC[C@H]4C#N)ccc3o2)c2cc(NC(=O)C3CC3)ncc12 ; CNc1ncc(-c2nc3cc(N4CC[C@H]4C(N)=O)ccc3o2)c2cc(NC(=O)C3CC3)ncc12 ; CNc1ncc(-c2nc3cc(N4CC5(CN(C(C)=O)C5)C4)ccc3o2)c2cc(NC(=O)C3CC3)ncc12]

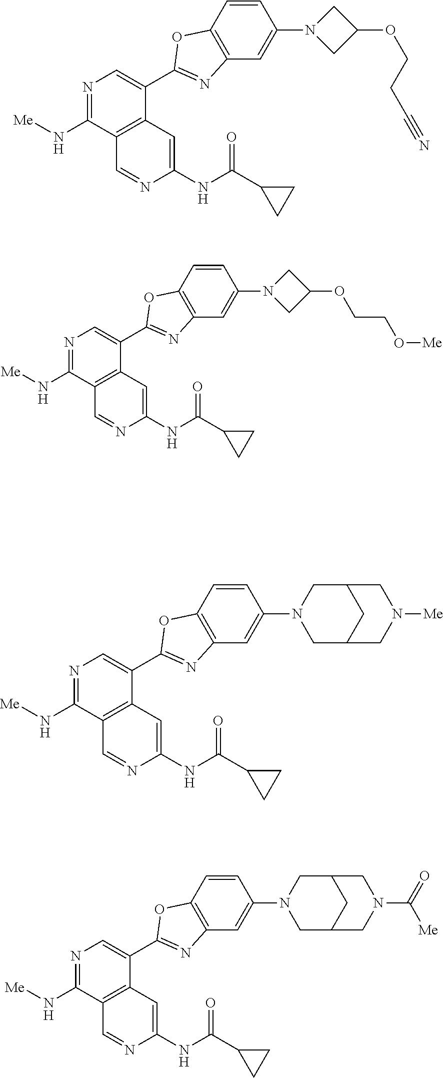 [SMILES: CNc1ncc(-c2nc3cc(N4CC(OCCC#N)C4)ccc3o2)c2cc(NC(=O)C3CC3)ncc12 ; CNc1ncc(-c2nc3cc(N4CC(OCCOC)C4)ccc3o2)c2cc(NC(=O)C3CC3)ncc12 ; CNc1ncc(-c2nc3cc(N4CC5CC(CN(C)C5)C4)ccc3o2)c2cc(NC(=O)C3CC3)ncc12 ; CNc1ncc(-c2nc3cc(N4CC5CC(CN(C(C)=O)C5)C4)ccc3o2)c2cc(NC(=O)C3CC3)ncc12]

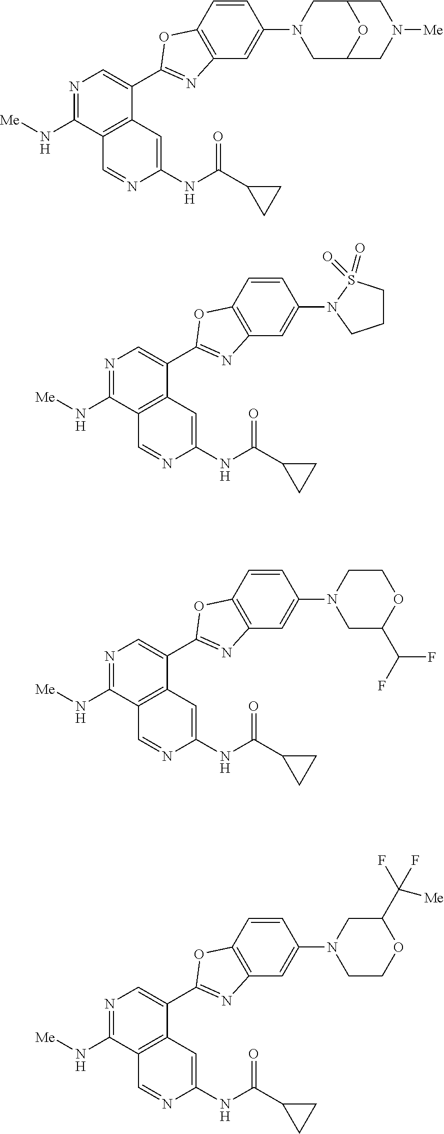 [SMILES: CNc1ncc(-c2nc3cc(N4CC5CN(C)CC(C4)O5)ccc3o2)c2cc(NC(=O)C3CC3)ncc12 ; CNc1ncc(-c2nc3cc(N4CCCS4(=O)=O)ccc3o2)c2cc(NC(=O)C3CC3)ncc12 ; CNc1ncc(-c2nc3cc(N4CCOC(C(F)F)C4)ccc3o2)c2cc(NC(=O)C3CC3)ncc12 ; CNc1ncc(-c2nc3cc(N4CCOC(C(C)(F)F)C4)ccc3o2)c2cc(NC(=O)C3CC3)ncc12]

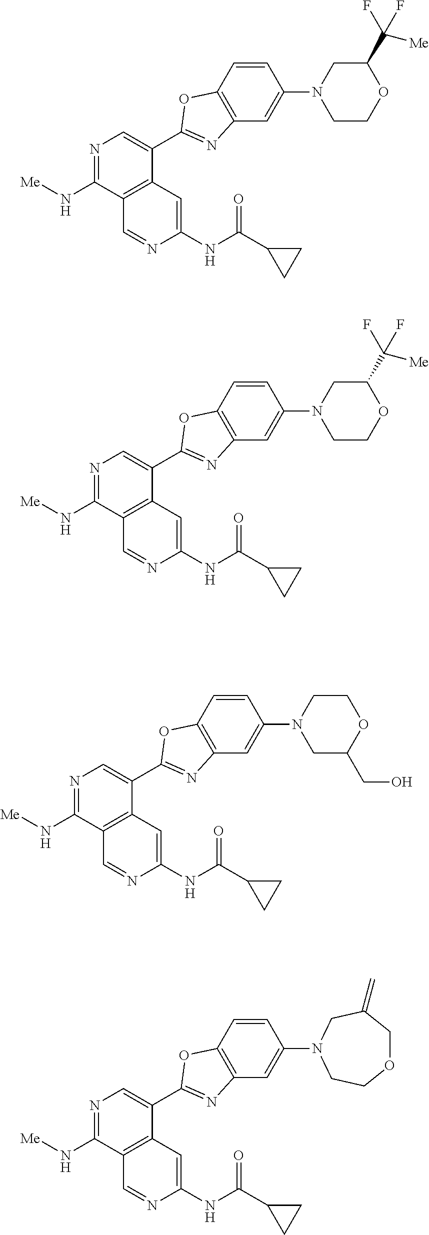 [SMILES: CNc1ncc(-c2nc3cc(N4CCO[C@H](C(C)(F)F)C4)ccc3o2)c2cc(NC(=O)C3CC3)ncc12 ; CNc1ncc(-c2nc3cc(N4CCO[C@@H](C(C)(F)F)C4)ccc3o2)c2cc(NC(=O)C3CC3)ncc12 ; CNc1ncc(-c2nc3cc(N4CCOC(CO)C4)ccc3o2)c2cc(NC(=O)C3CC3)ncc12 ; C=C1COCCN(c2ccc3oc(-c4cnc(NC)c5cnc(NC(=O)C6CC6)cc45)nc3c2)C1]

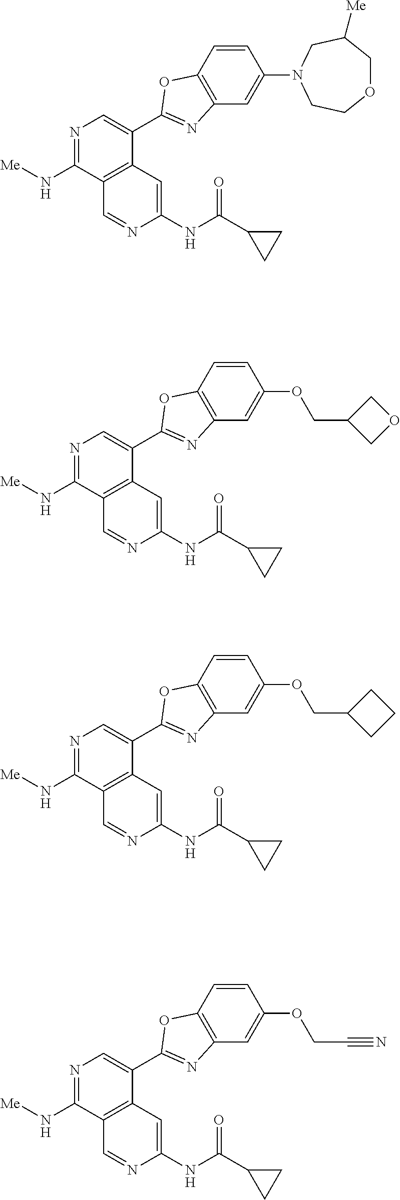 [SMILES: CNc1ncc(-c2nc3cc(N4CCOCC(C)C4)ccc3o2)c2cc(NC(=O)C3CC3)ncc12 ; CNc1ncc(-c2nc3cc(OCC4COC4)ccc3o2)c2cc(NC(=O)C3CC3)ncc12 ; CNc1ncc(-c2nc3cc(OCC4CCC4)ccc3o2)c2cc(NC(=O)C3CC3)ncc12 ; CNc1ncc(-c2nc3cc(OCC#N)ccc3o2)c2cc(NC(=O)C3CC3)ncc12]

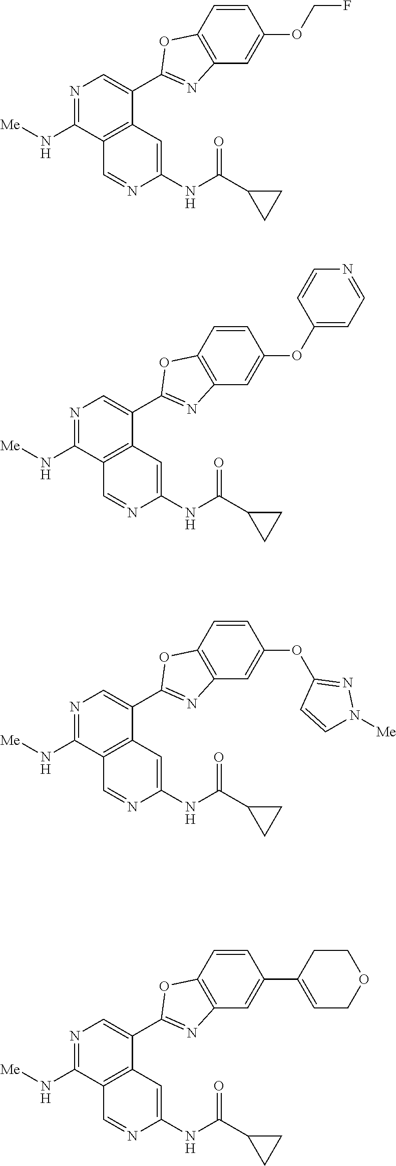 [SMILES: CNc1ncc(-c2nc3cc(OCF)ccc3o2)c2cc(NC(=O)C3CC3)ncc12 ; CNc1ncc(-c2nc3cc(Oc4ccncc4)ccc3o2)c2cc(NC(=O)C3CC3)ncc12 ; CNc1ncc(-c2nc3cc(Oc4ccn(C)n4)ccc3o2)c2cc(NC(=O)C3CC3)ncc12 ; CNc1ncc(-c2nc3cc(C4=CCOCC4)ccc3o2)c2cc(NC(=O)C3CC3)ncc12]

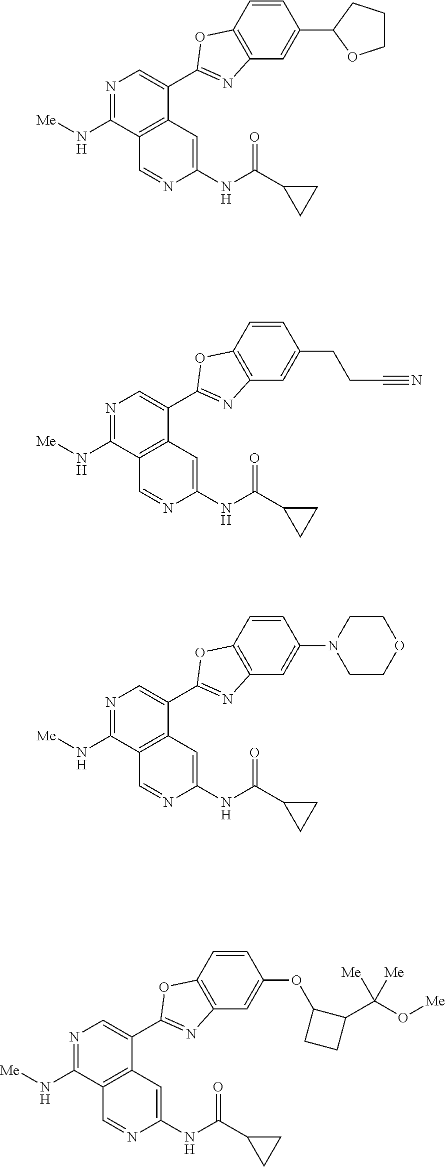 [SMILES: CNc1ncc(-c2nc3cc(C4CCCO4)ccc3o2)c2cc(NC(=O)C3CC3)ncc12 ; CNc1ncc(-c2nc3cc(CCC#N)ccc3o2)c2cc(NC(=O)C3CC3)ncc12 ; CNc1ncc(-c2nc3cc(N4CCOCC4)ccc3o2)c2cc(NC(=O)C3CC3)ncc12 ; CNc1ncc(-c2nc3cc(OC4CCC4C(C)(C)OC)ccc3o2)c2cc(NC(=O)C3CC3)ncc12]

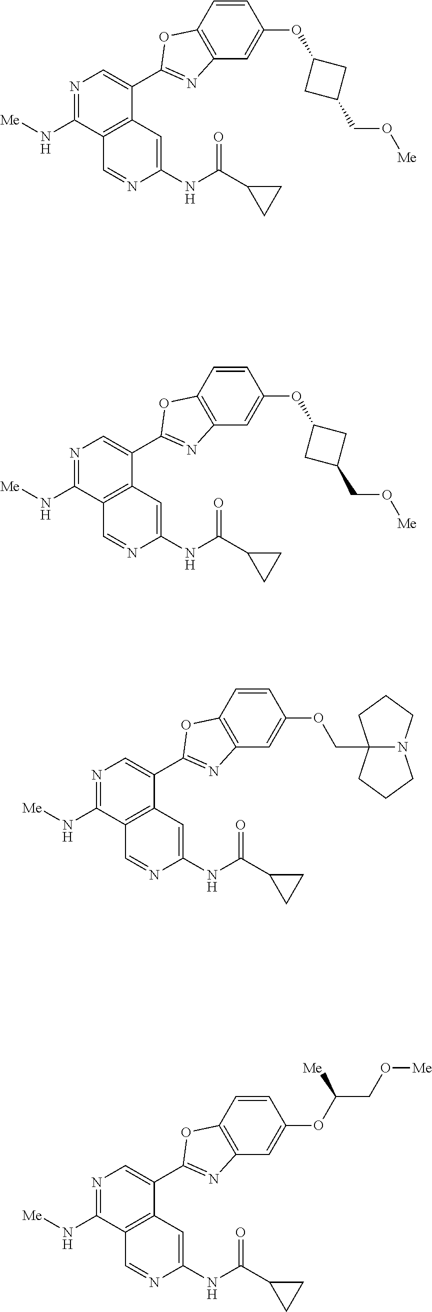 [SMILES: CNc1ncc(-c2nc3cc(O[C@H]4C[C@@H](COC)C4)ccc3o2)c2cc(NC(=O)C3CC3)ncc12 ; CNc1ncc(-c2nc3cc(O[C@H]4C[C@H](COC)C4)ccc3o2)c2cc(NC(=O)C3CC3)ncc12 ; CNc1ncc(-c2nc3cc(OCC45CCCN4CCC5)ccc3o2)c2cc(NC(=O)C3CC3)ncc12 ; CNc1ncc(-c2nc3cc(O[C@@H](C)COC)ccc3o2)c2cc(NC(=O)C3CC3)ncc12]

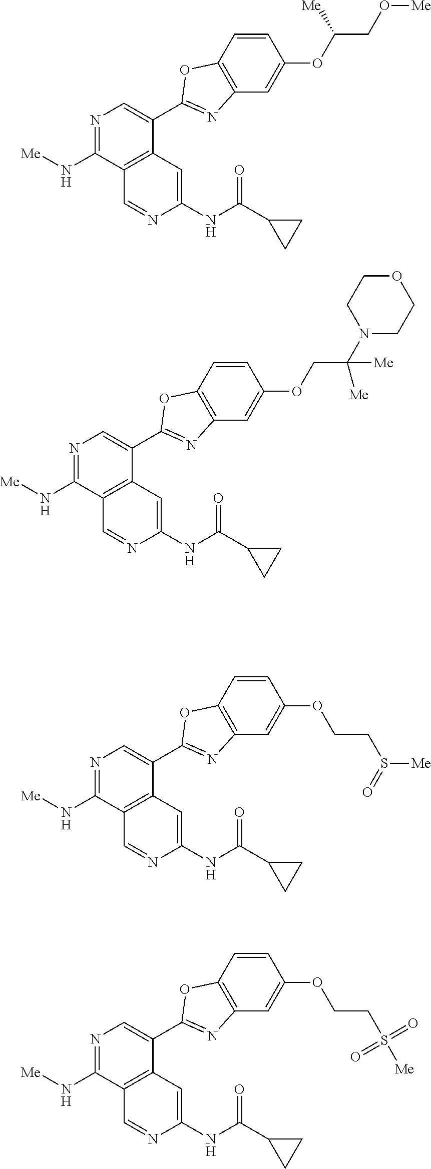 [SMILES: CNc1ncc(-c2nc3cc(O[C@H](C)COC)ccc3o2)c2cc(NC(=O)C3CC3)ncc12 ; CNc1ncc(-c2nc3cc(OCC(C)(C)N4CCOCC4)ccc3o2)c2cc(NC(=O)C3CC3)ncc12 ; CNc1ncc(-c2nc3cc(OCCS(C)=O)ccc3o2)c2cc(NC(=O)C3CC3)ncc12 ; CNc1ncc(-c2nc3cc(OCCS(C)(=O)=O)ccc3o2)c2cc(NC(=O)C3CC3)ncc12]

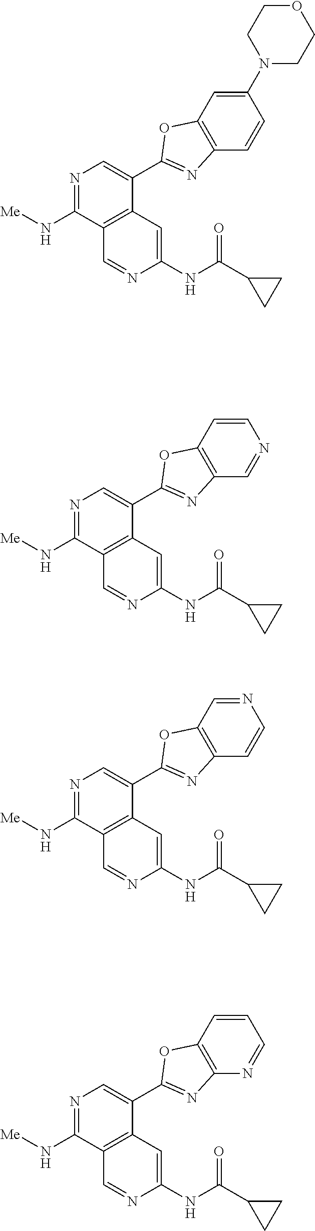 [SMILES: CNc1ncc(-c2nc3ccc(N4CCOCC4)cc3o2)c2cc(NC(=O)C3CC3)ncc12 ; CNc1ncc(-c2nc3cnccc3o2)c2cc(NC(=O)C3CC3)ncc12 ; CNc1ncc(-c2nc3ccncc3o2)c2cc(NC(=O)C3CC3)ncc12 ; CNc1ncc(-c2nc3ncccc3o2)c2cc(NC(=O)C3CC3)ncc12]

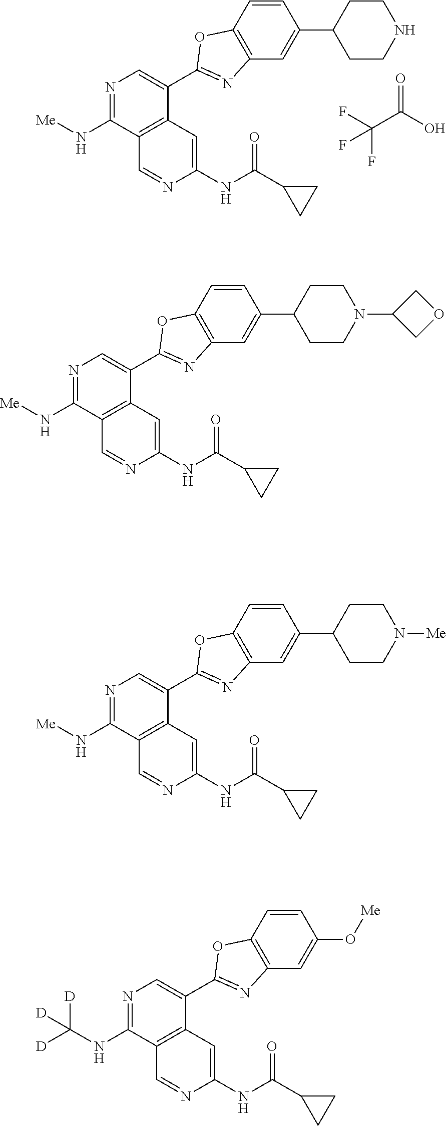

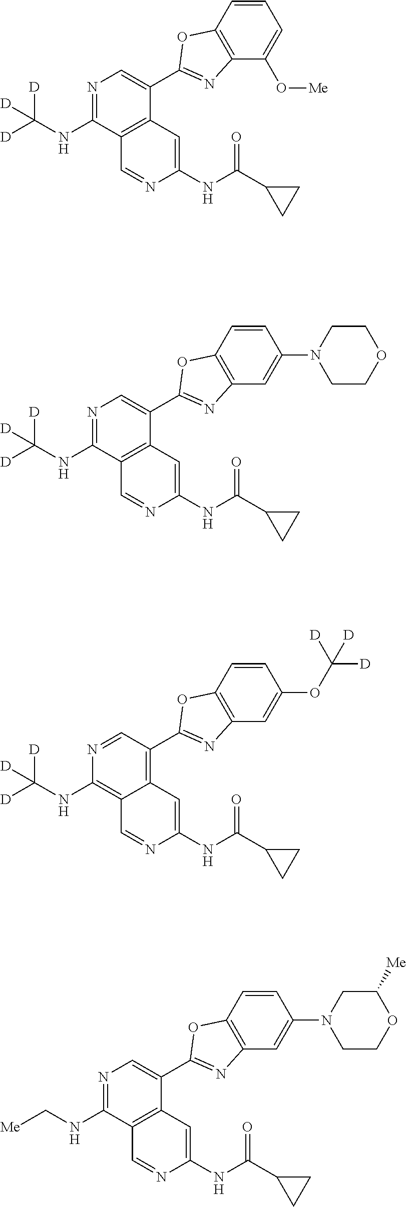 [SMILES: [2H]C([2H])([2H])Nc1ncc(-c2nc3c(OC)cccc3o2)c2cc(NC(=O)C3CC3)ncc12 ; [2H]C([2H])([2H])Nc1ncc(-c2nc3cc(N4CCOCC4)ccc3o2)c2cc(NC(=O)C3CC3)ncc12 ; [2H]C([2H])([2H])Nc1ncc(-c2nc3cc(OC([2H])([2H])[2H])ccc3o2)c2cc(NC(=O)C3CC3)ncc12 ; CCNc1ncc(-c2nc3cc(N4CCO[C@@H](C)C4)ccc3o2)c2cc(NC(=O)C3CC3)ncc12]

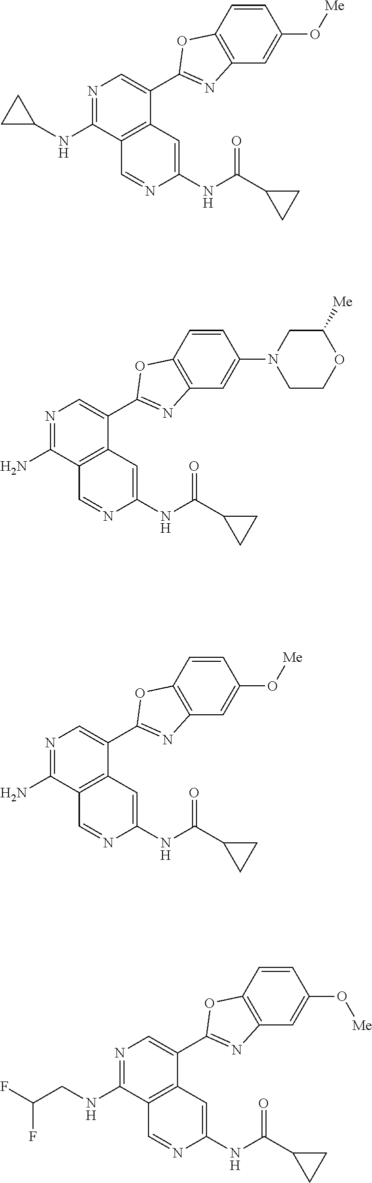 [SMILES: COc1ccc2oc(-c3cnc(NC4CC4)c4cnc(NC(=O)C5CC5)cc34)nc2c1 ; C[C@H]1CN(c2ccc3oc(-c4cnc(N)c5cnc(NC(=O)C6CC6)cc45)nc3c2)CCO1 ; COc1ccc2oc(-c3cnc(N)c4cnc(NC(=O)C5CC5)cc34)nc2c1 ; COc1ccc2oc(-c3cnc(NCC(F)F)c4cnc(NC(=O)C5CC5)cc34)nc2c1]

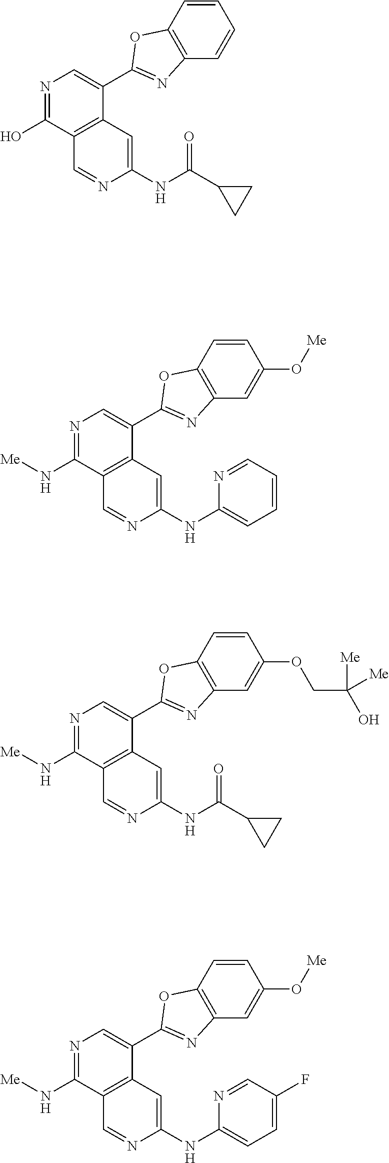 [SMILES: O=C(Nc1cc2c(-c3nc4ccccc4o3)cnc(O)c2cn1)C1CC1 ; CNc1ncc(-c2nc3cc(OC)ccc3o2)c2cc(Nc3ccccn3)ncc12 ; CNc1ncc(-c2nc3cc(OCC(C)(C)O)ccc3o2)c2cc(NC(=O)C3CC3)ncc12 ; CNc1ncc(-c2nc3cc(OC)ccc3o2)c2cc(Nc3ccc(F)cn3)ncc12]

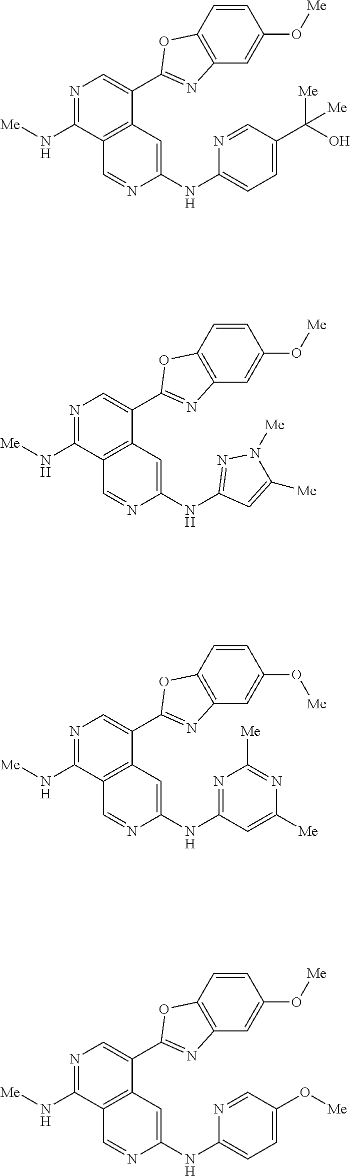 [SMILES: CNc1ncc(-c2nc3cc(OC)ccc3o2)c2cc(Nc3ccc(C(C)(C)O)cn3)ncc12 ; CNc1ncc(-c2nc3cc(OC)ccc3o2)c2cc(Nc3cc(C)n(C)n3)ncc12 ; CNc1ncc(-c2nc3cc(OC)ccc3o2)c2cc(Nc3cc(C)nc(C)n3)ncc12 ; CNc1ncc(-c2nc3cc(OC)ccc3o2)c2cc(Nc3ccc(OC)cn3)ncc12]

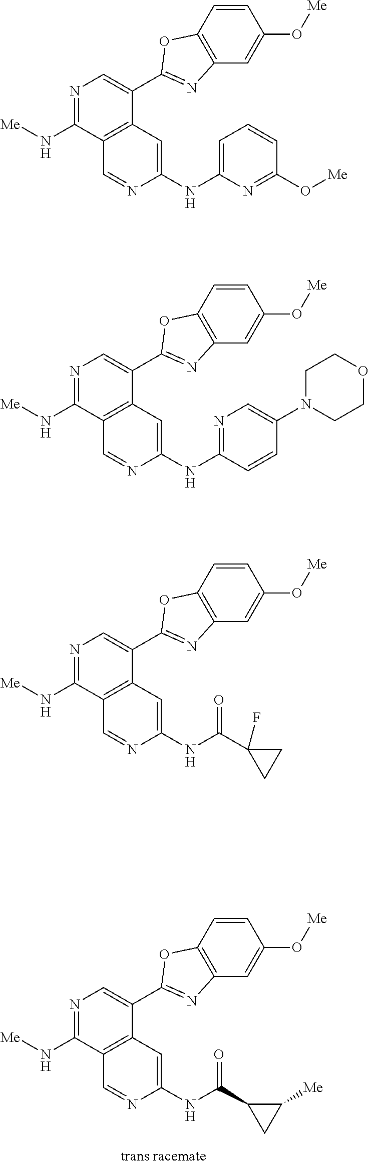 [SMILES: CNc1ncc(-c2nc3cc(OC)ccc3o2)c2cc(Nc3cccc(OC)n3)ncc12 ; CNc1ncc(-c2nc3cc(OC)ccc3o2)c2cc(Nc3ccc(N4CCOCC4)cn3)ncc12 ; CNc1ncc(-c2nc3cc(OC)ccc3o2)c2cc(NC(=O)C3(F)CC3)ncc12 ; CNc1ncc(-c2nc3cc(OC)ccc3o2)c2cc(NC(=O)[C@@H]3C[C@H]3C)ncc12]

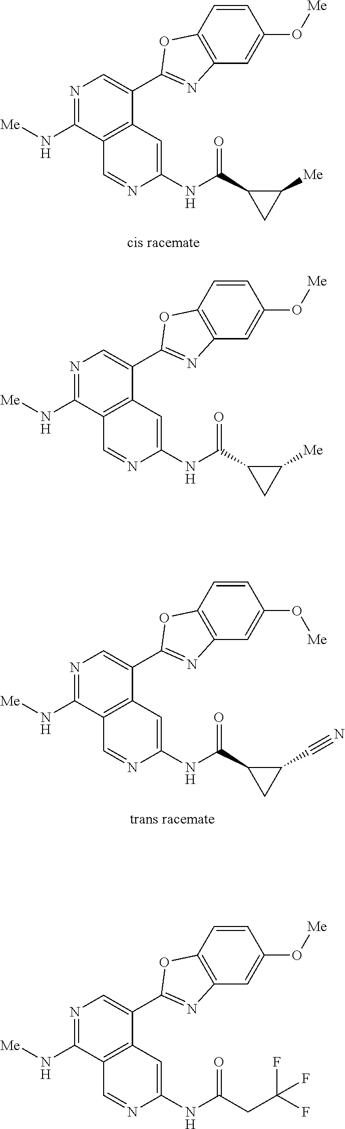 [SMILES: CNc1ncc(-c2nc3cc(OC)ccc3o2)c2cc(NC(=O)[C@@H]3C[C@@H]3C)ncc12 ; CNc1ncc(-c2nc3cc(OC)ccc3o2)c2cc(NC(=O)[C@H]3C[C@H]3C)ncc12 ; CNc1ncc(-c2nc3cc(OC)ccc3o2)c2cc(NC(=O)[C@@H]3C[C@H]3C#N)ncc12 ; CNc1ncc(-c2nc3cc(OC)ccc3o2)c2cc(NC(=O)CC(F)(F)F)ncc12]

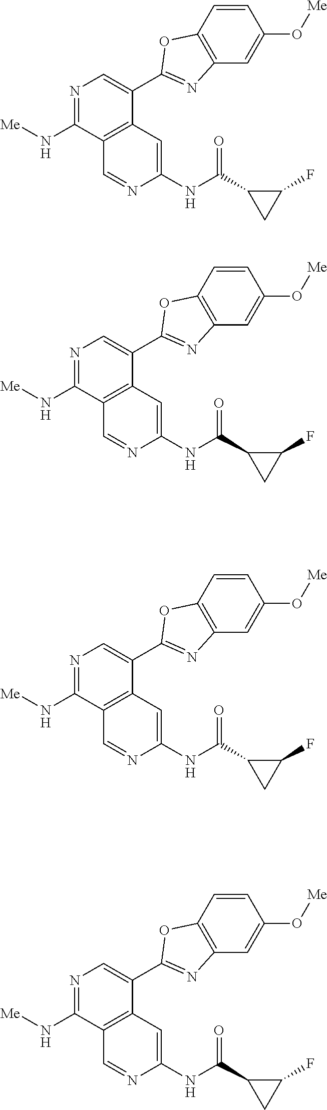 [SMILES: CNc1ncc(-c2nc3cc(OC)ccc3o2)c2cc(NC(=O)[C@H]3C[C@H]3F)ncc12 ; CNc1ncc(-c2nc3cc(OC)ccc3o2)c2cc(NC(=O)[C@@H]3C[C@@H]3F)ncc12 ; CNc1ncc(-c2nc3cc(OC)ccc3o2)c2cc(NC(=O)[C@H]3C[C@@H]3F)ncc12 ; CNc1ncc(-c2nc3cc(OC)ccc3o2)c2cc(NC(=O)[C@@H]3C[C@H]3F)ncc12]

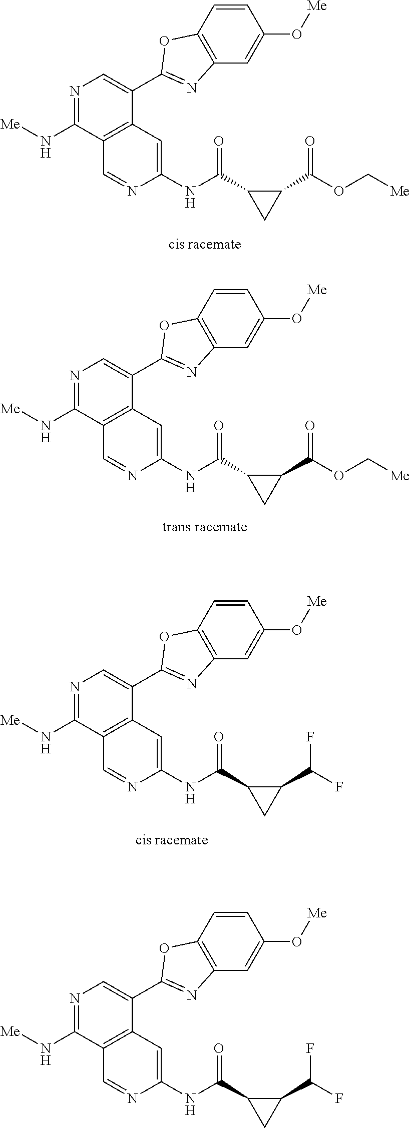 [SMILES: CCOC(=O)[C@@H]1C[C@@H]1C(=O)Nc1cc2c(-c3nc4cc(OC)ccc4o3)cnc(NC)c2cn1 ; CCOC(=O)[C@H]1C[C@@H]1C(=O)Nc1cc2c(-c3nc4cc(OC)ccc4o3)cnc(NC)c2cn1 ; CNc1ncc(-c2nc3cc(OC)ccc3o2)c2cc(NC(=O)[C@@H]3C[C@@H]3C(F)F)ncc12 ; CNc1ncc(-c2nc3cc(OC)ccc3o2)c2cc(NC(=O)[C@@H]3C[C@@H]3C(F)F)ncc12]

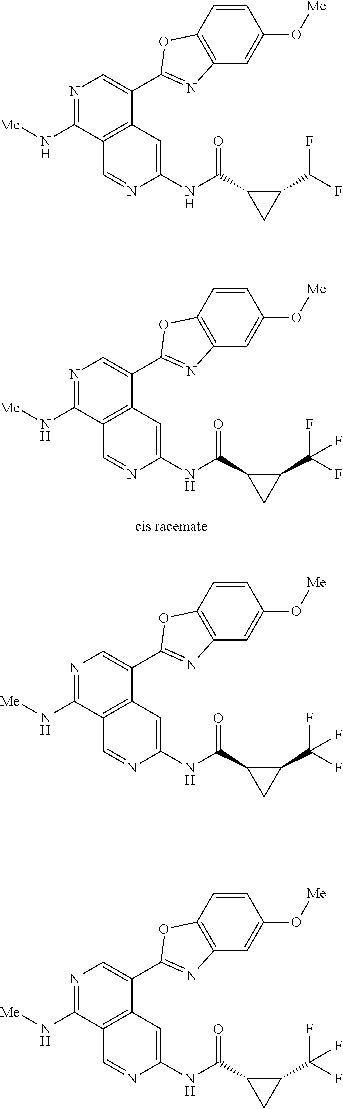 [SMILES: CNc1ncc(-c2nc3cc(OC)ccc3o2)c2cc(NC(=O)[C@H]3C[C@H]3C(F)F)ncc12 ; CNc1ncc(-c2nc3cc(OC)ccc3o2)c2cc(NC(=O)[C@@H]3C[C@@H]3C(F)(F)F)ncc12 ; CNc1ncc(-c2nc3cc(OC)ccc3o2)c2cc(NC(=O)[C@@H]3C[C@@H]3C(F)(F)F)ncc12 ; CNc1ncc(-c2nc3cc(OC)ccc3o2)c2cc(NC(=O)[C@H]3C[C@H]3C(F)(F)F)ncc12]

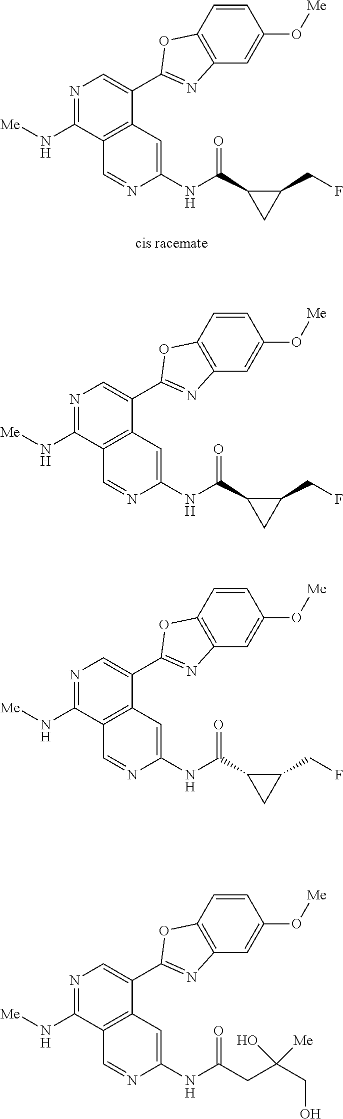 [SMILES: CNc1ncc(-c2nc3cc(OC)ccc3o2)c2cc(NC(=O)[C@@H]3C[C@@H]3CF)ncc12 ; CNc1ncc(-c2nc3cc(OC)ccc3o2)c2cc(NC(=O)[C@@H]3C[C@@H]3CF)ncc12 ; CNc1ncc(-c2nc3cc(OC)ccc3o2)c2cc(NC(=O)[C@H]3C[C@H]3CF)ncc12 ; CNc1ncc(-c2nc3cc(OC)ccc3o2)c2cc(NC(=O)CC(C)(O)CO)ncc12]

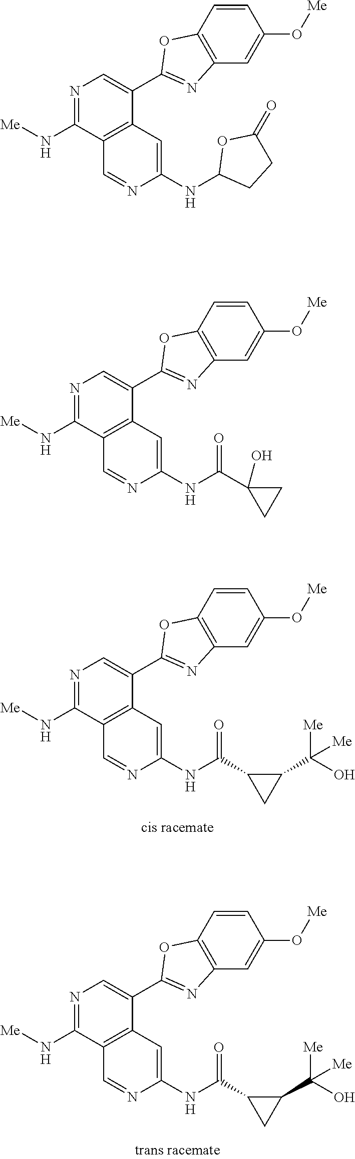 [SMILES: CNc1ncc(-c2nc3cc(OC)ccc3o2)c2cc(NC3CCC(=O)O3)ncc12 ; CNc1ncc(-c2nc3cc(OC)ccc3o2)c2cc(NC(=O)C3(O)CC3)ncc12 ; CNc1ncc(-c2nc3cc(OC)ccc3o2)c2cc(NC(=O)[C@H]3C[C@H]3C(C)(C)O)ncc12 ; CNc1ncc(-c2nc3cc(OC)ccc3o2)c2cc(NC(=O)[C@H]3C[C@@H]3C(C)(C)O)ncc12]

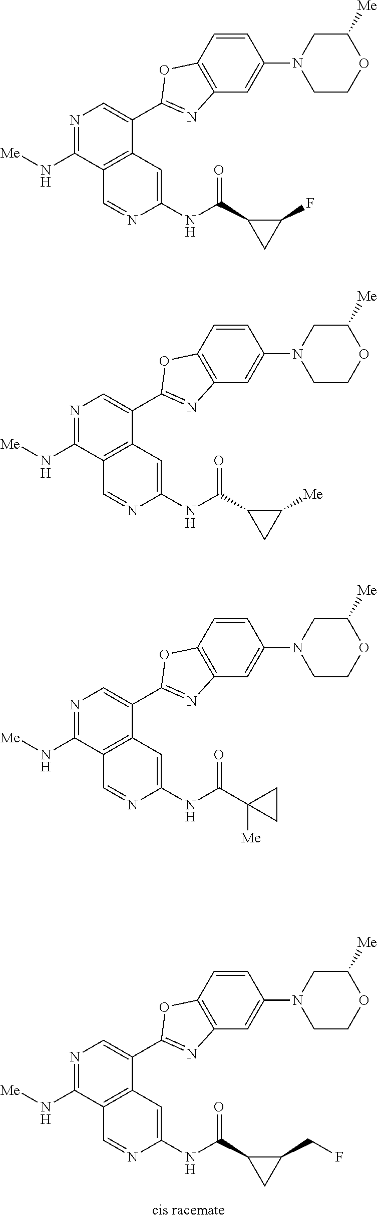 [SMILES: CNc1ncc(-c2nc3cc(N4CCO[C@@H](C)C4)ccc3o2)c2cc(NC(=O)[C@@H]3C[C@@H]3F)ncc12 ; CNc1ncc(-c2nc3cc(N4CCO[C@@H](C)C4)ccc3o2)c2cc(NC(=O)[C@H]3C[C@H]3C)ncc12 ; CNc1ncc(-c2nc3cc(N4CCO[C@@H](C)C4)ccc3o2)c2cc(NC(=O)C3(C)CC3)ncc12 ; CNc1ncc(-c2nc3cc(N4CCO[C@@H](C)C4)ccc3o2)c2cc(NC(=O)[C@@H]3C[C@@H]3CF)ncc12]

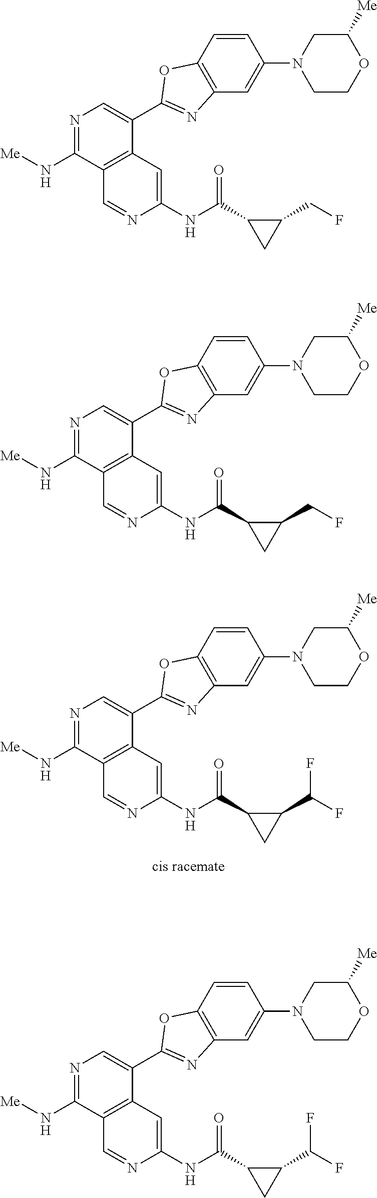 [SMILES: CNc1ncc(-c2nc3cc(N4CCO[C@@H](C)C4)ccc3o2)c2cc(NC(=O)[C@H]3C[C@H]3CF)ncc12 ; CNc1ncc(-c2nc3cc(N4CCO[C@@H](C)C4)ccc3o2)c2cc(NC(=O)[C@@H]3C[C@@H]3CF)ncc12 ; CNc1ncc(-c2nc3cc(N4CCO[C@@H](C)C4)ccc3o2)c2cc(NC(=O)[C@@H]3C[C@@H]3C(F)F)ncc12 ; CNc1ncc(-c2nc3cc(N4CCO[C@@H](C)C4)ccc3o2)c2cc(NC(=O)[C@H]3C[C@H]3C(F)F)ncc12]

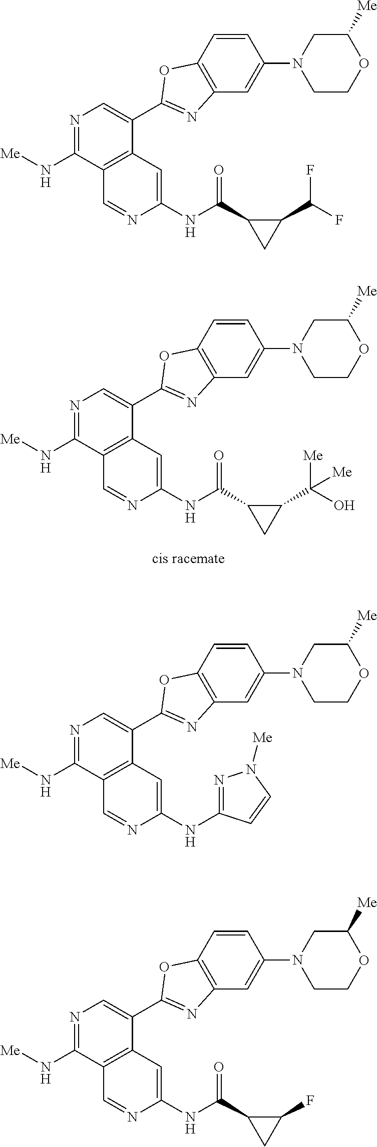 [SMILES: CNc1ncc(-c2nc3cc(N4CCO[C@@H](C)C4)ccc3o2)c2cc(NC(=O)[C@@H]3C[C@@H]3C(F)F)ncc12 ; CNc1ncc(-c2nc3cc(N4CCO[C@@H](C)C4)ccc3o2)c2cc(NC(=O)[C@H]3C[C@H]3C(C)(C)O)ncc12 ; CNc1ncc(-c2nc3cc(N4CCO[C@@H](C)C4)ccc3o2)c2cc(Nc3ccn(C)n3)ncc12 ; CNc1ncc(-c2nc3cc(N4CCO[C@H](C)C4)ccc3o2)c2cc(NC(=O)[C@@H]3C[C@@H]3F)ncc12]

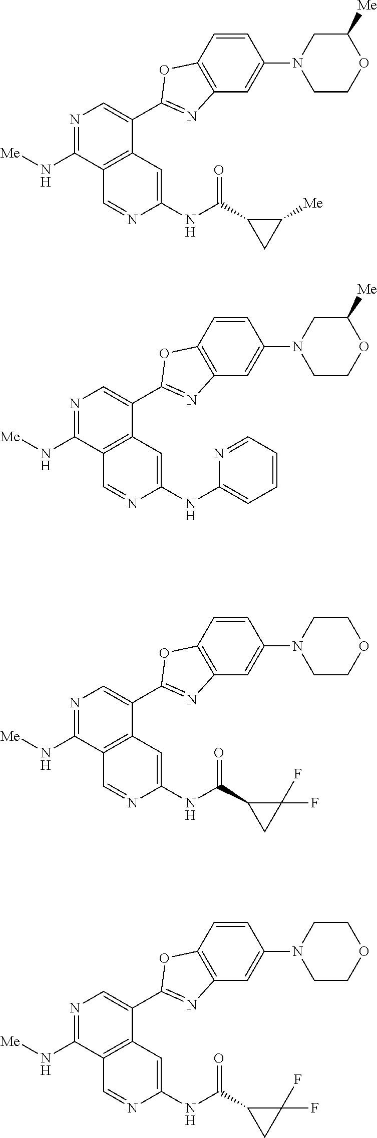 [SMILES: CNc1ncc(-c2nc3cc(N4CCO[C@H](C)C4)ccc3o2)c2cc(NC(=O)[C@H]3C[C@H]3C)ncc12 ; CNc1ncc(-c2nc3cc(N4CCO[C@H](C)C4)ccc3o2)c2cc(Nc3ccccn3)ncc12 ; CNc1ncc(-c2nc3cc(N4CCOCC4)ccc3o2)c2cc(NC(=O)[C@@H]3CC3(F)F)ncc12 ; CNc1ncc(-c2nc3cc(N4CCOCC4)ccc3o2)c2cc(NC(=O)[C@H]3CC3(F)F)ncc12]

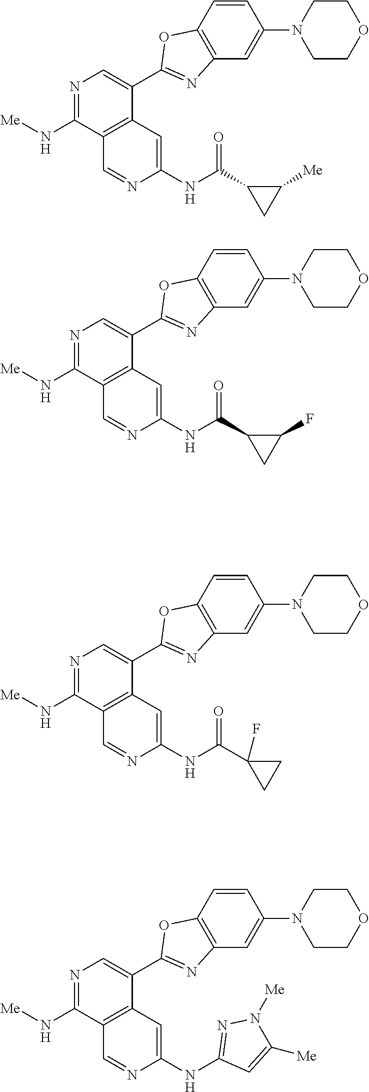

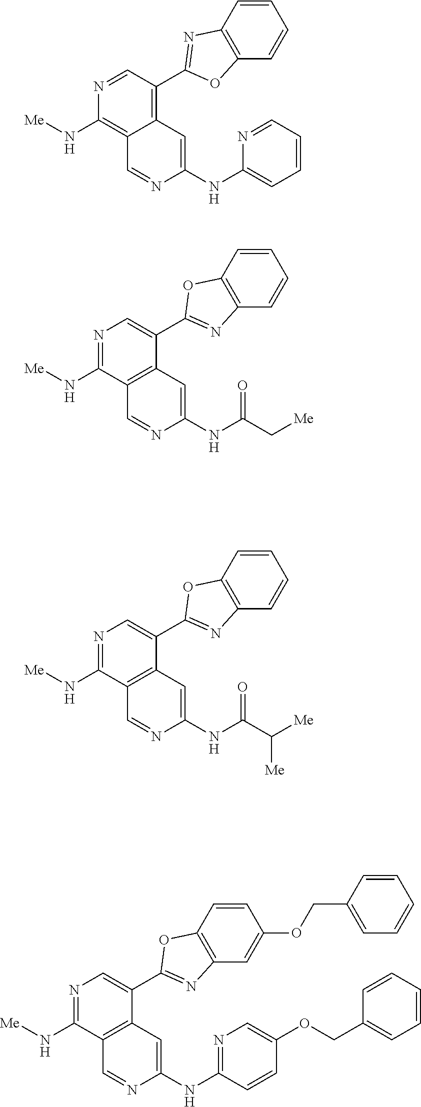 [SMILES: CNc1ncc(-c2nc3ccccc3o2)c2cc(Nc3ccccn3)ncc12 ; CCC(=O)Nc1cc2c(-c3nc4ccccc4o3)cnc(NC)c2cn1 ; CNc1ncc(-c2nc3ccccc3o2)c2cc(NC(=O)C(C)C)ncc12 ; CNc1ncc(-c2nc3cc(OCc4ccccc4)ccc3o2)c2cc(Nc3ccc(OCc4ccccc4)cn3)ncc12]

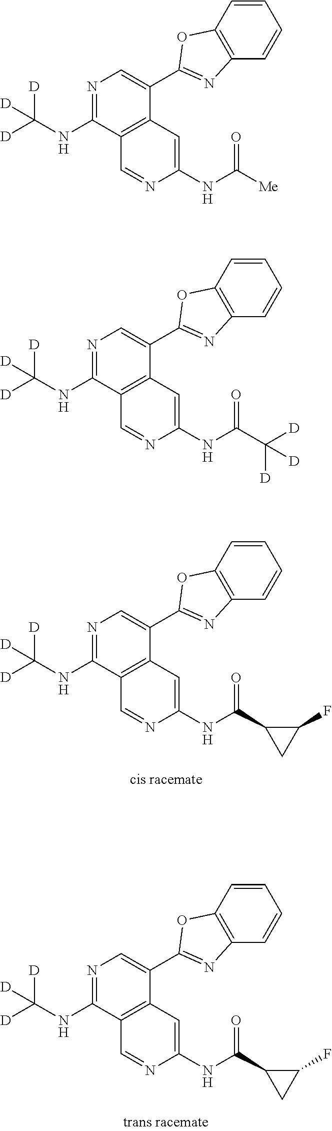 [SMILES: [2H]C([2H])([2H])Nc1ncc(-c2nc3ccccc3o2)c2cc(NC(C)=O)ncc12 ; [2H]C([2H])([2H])Nc1ncc(-c2nc3ccccc3o2)c2cc(NC(=O)C([2H])([2H])[2H])ncc12 ; [2H]C([2H])([2H])Nc1ncc(-c2nc3ccccc3o2)c2cc(NC(=O)[C@@H]3C[C@@H]3F)ncc12 ; [2H]C([2H])([2H])Nc1ncc(-c2nc3ccccc3o2)c2cc(NC(=O)[C@@H]3C[C@H]3F)ncc12]

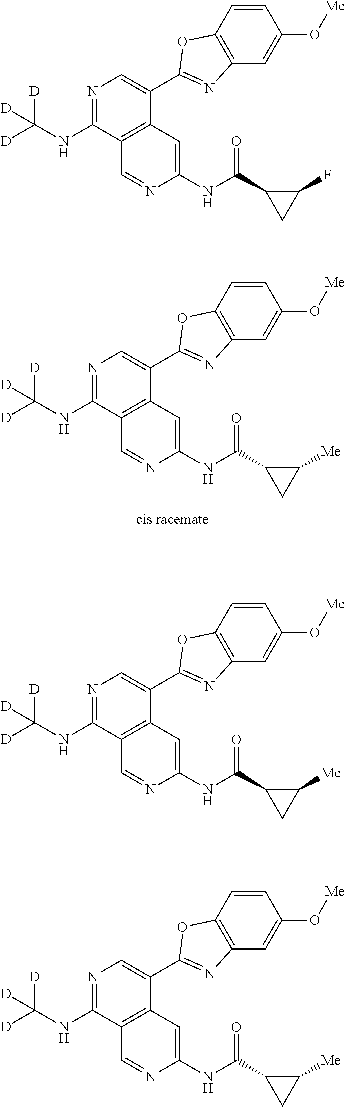 [SMILES: [2H]C([2H])([2H])Nc1ncc(-c2nc3cc(OC)ccc3o2)c2cc(NC(=O)[C@@H]3C[C@@H]3F)ncc12 ; [2H]C([2H])([2H])Nc1ncc(-c2nc3cc(OC)ccc3o2)c2cc(NC(=O)[C@H]3C[C@H]3C)ncc12 ; [2H]C([2H])([2H])Nc1ncc(-c2nc3cc(OC)ccc3o2)c2cc(NC(=O)[C@@H]3C[C@@H]3C)ncc12 ; [2H]C([2H])([2H])Nc1ncc(-c2nc3cc(OC)ccc3o2)c2cc(NC(=O)[C@H]3C[C@H]3C)ncc12]

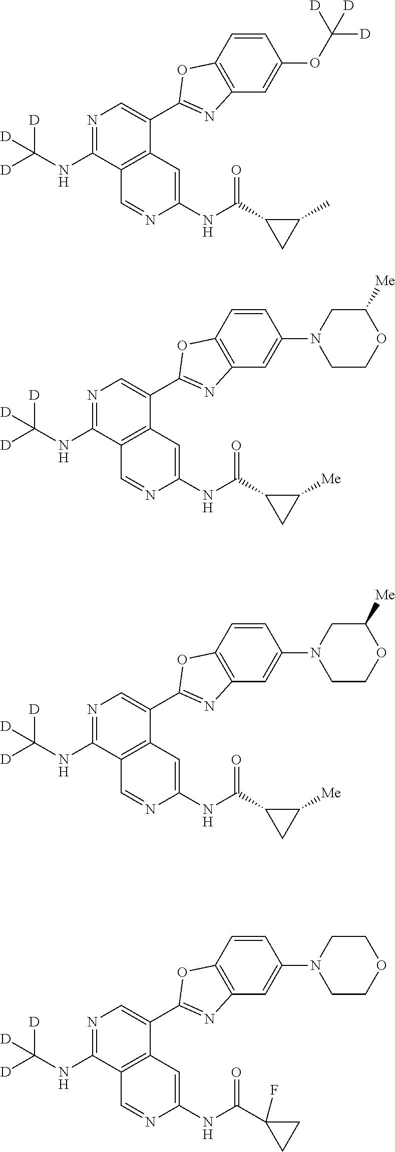 [SMILES: [2H]C([2H])([2H])Nc1ncc(-c2nc3cc(OC([2H])([2H])[2H])ccc3o2)c2cc(NC(=O)[C@H]3C[C@H]3C)ncc12 ; [2H]C([2H])([2H])Nc1ncc(-c2nc3cc(N4CCO[C@@H](C)C4)ccc3o2)c2cc(NC(=O)[C@H]3C[C@H]3C)ncc12 ; [2H]C([2H])([2H])Nc1ncc(-c2nc3cc(N4CCO[C@H](C)C4)ccc3o2)c2cc(NC(=O)[C@H]3C[C@H]3C)ncc12 ; [2H]C([2H])([2H])Nc1ncc(-c2nc3cc(N4CCOCC4)ccc3o2)c2cc(NC(=O)C3(F)CC3)ncc12]

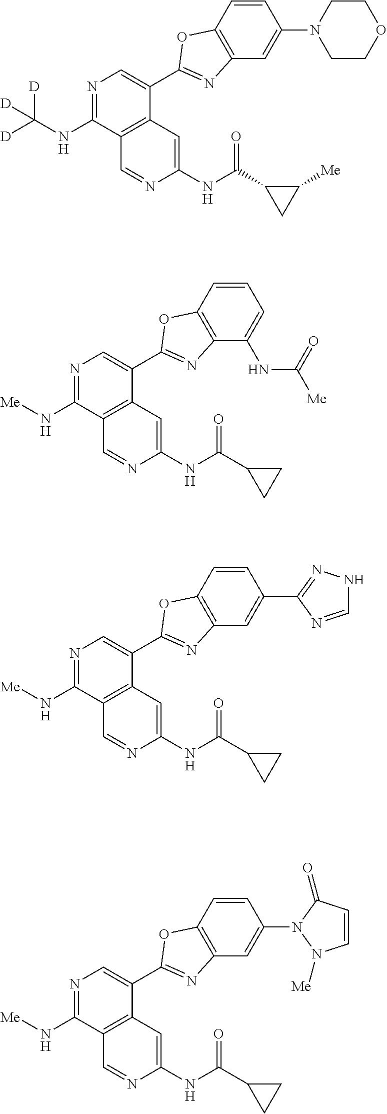 [SMILES: [2H]C([2H])([2H])Nc1ncc(-c2nc3cc(N4CCOCC4)ccc3o2)c2cc(NC(=O)[C@H]3C[C@H]3C)ncc12 ; CNc1ncc(-c2nc3c(NC(C)=O)cccc3o2)c2cc(NC(=O)C3CC3)ncc12 ; CNc1ncc(-c2nc3cc(-c4nc[nH]n4)ccc3o2)c2cc(NC(=O)C3CC3)ncc12 ; CNc1ncc(-c2nc3cc(-n4c(=O)ccn4C)ccc3o2)c2cc(NC(=O)C3CC3)ncc12]

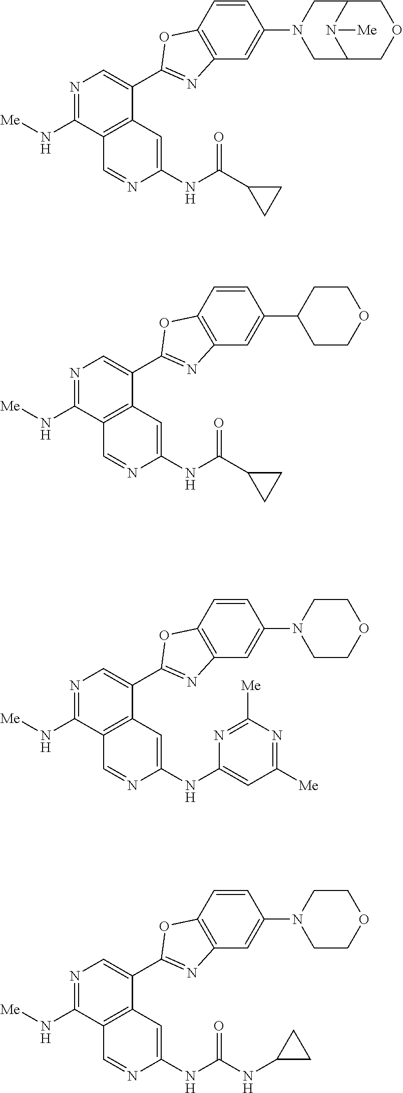 [SMILES: CNc1ncc(-c2nc3cc(N4CC5COCC(C4)N5C)ccc3o2)c2cc(NC(=O)C3CC3)ncc12 ; CNc1ncc(-c2nc3cc(C4CCOCC4)ccc3o2)c2cc(NC(=O)C3CC3)ncc12 ; CNc1ncc(-c2nc3cc(N4CCOCC4)ccc3o2)c2cc(Nc3cc(C)nc(C)n3)ncc12 ; CNc1ncc(-c2nc3cc(N4CCOCC4)ccc3o2)c2cc(NC(=O)NC3CC3)ncc12]

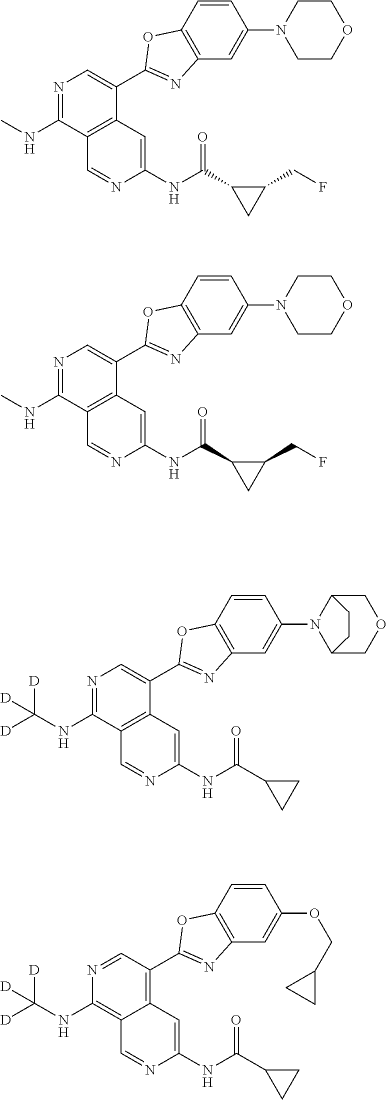 [SMILES: CNc1ncc(-c2nc3cc(N4CCOCC4)ccc3o2)c2cc(NC(=O)[C@H]3C[C@H]3CF)ncc12 ; CNc1ncc(-c2nc3cc(N4CCOCC4)ccc3o2)c2cc(NC(=O)[C@@H]3C[C@@H]3CF)ncc12 ; [2H]C([2H])([2H])Nc1ncc(-c2nc3cc(N4C5CCC4COC5)ccc3o2)c2cc(NC(=O)C3CC3)ncc12 ; [2H]C([2H])([2H])Nc1ncc(-c2nc3cc(OCC4CC4)ccc3o2)c2cc(NC(=O)C3CC3)ncc12]

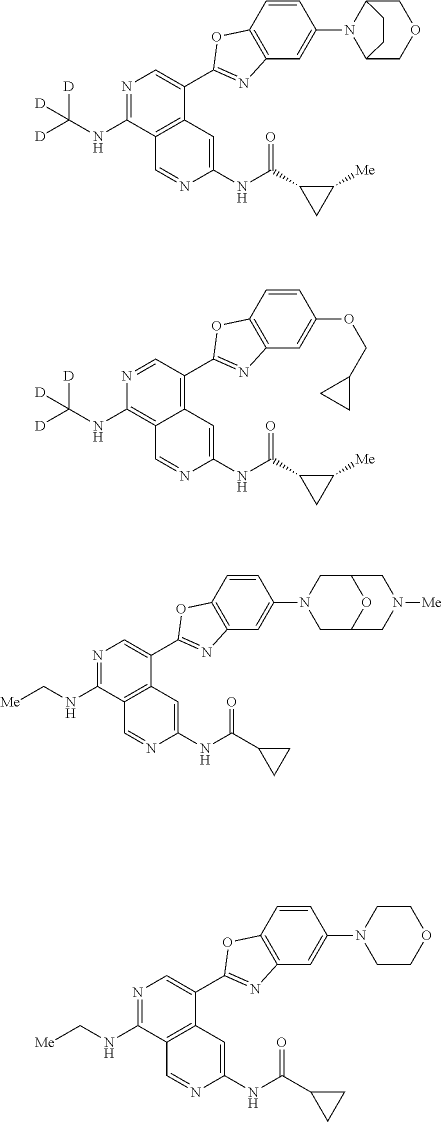 [SMILES: [2H]C([2H])([2H])Nc1ncc(-c2nc3cc(N4C5CCC4COC5)ccc3o2)c2cc(NC(=O)[C@H]3C[C@H]3C)ncc12 ; [2H]C([2H])([2H])Nc1ncc(-c2nc3cc(OCC4CC4)ccc3o2)c2cc(NC(=O)[C@H]3C[C@H]3C)ncc12 ; CCNc1ncc(-c2nc3cc(N4CC5CN(C)CC(C4)O5)ccc3o2)c2cc(NC(=O)C3CC3)ncc12 ; CCNc1ncc(-c2nc3cc(N4CCOCC4)ccc3o2)c2cc(NC(=O)C3CC3)ncc12]

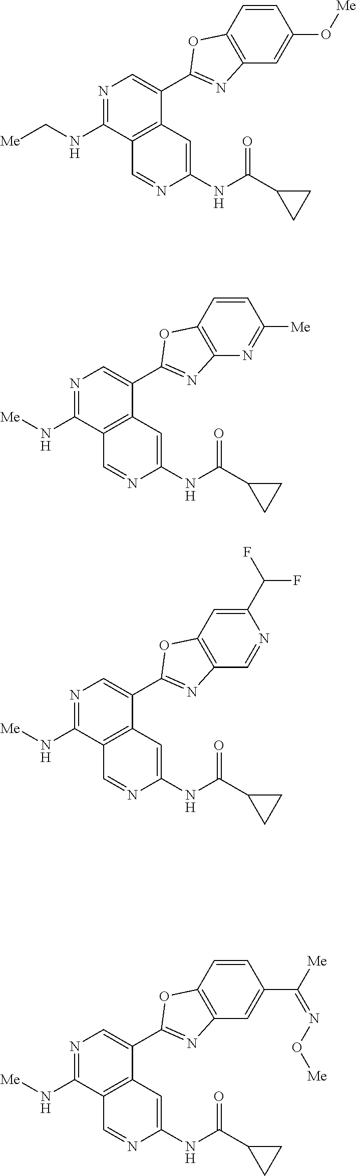

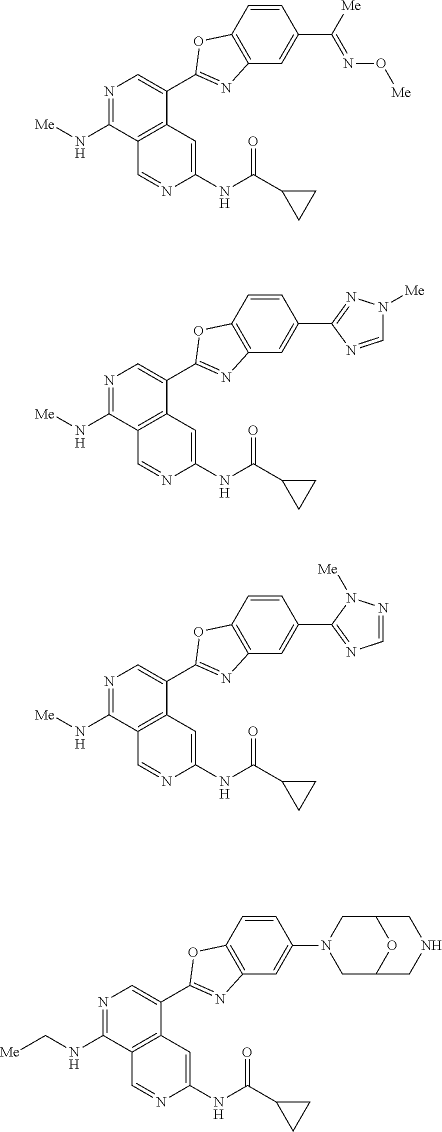 [SMILES: CNc1ncc(-c2nc3cc(/C(C)=N/OC)ccc3o2)c2cc(NC(=O)C3CC3)ncc12 ; CNc1ncc(-c2nc3cc(-c4ncn(C)n4)ccc3o2)c2cc(NC(=O)C3CC3)ncc12 ; CNc1ncc(-c2nc3cc(-c4ncnn4C)ccc3o2)c2cc(NC(=O)C3CC3)ncc12 ; CCNc1ncc(-c2nc3cc(N4CC5CNCC(C4)O5)ccc3o2)c2cc(NC(=O)C3CC3)ncc12]

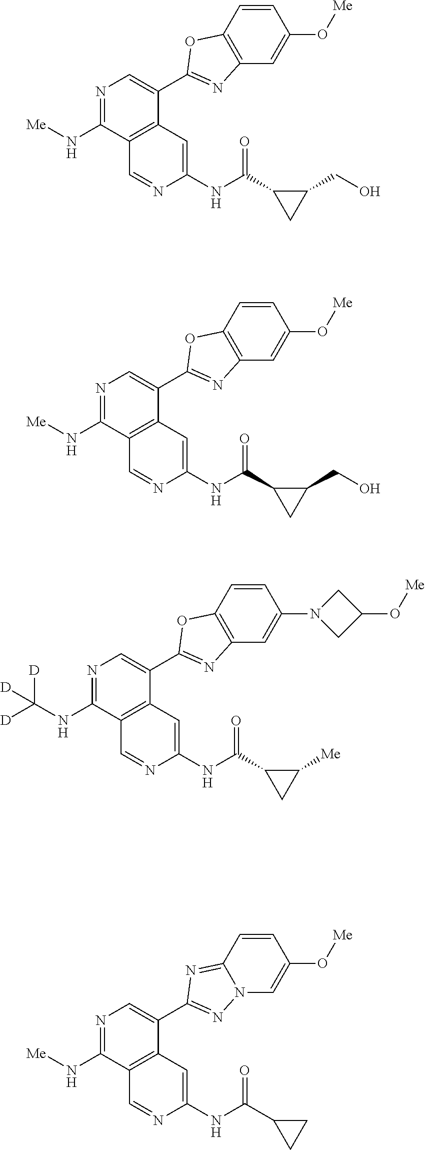 [SMILES: CNc1ncc(-c2nc3cc(OC)ccc3o2)c2cc(NC(=O)[C@H]3C[C@H]3CO)ncc12 ; CNc1ncc(-c2nc3cc(OC)ccc3o2)c2cc(NC(=O)[C@@H]3C[C@@H]3CO)ncc12 ; [2H]C([2H])([2H])Nc1ncc(-c2nc3cc(N4CC(OC)C4)ccc3o2)c2cc(NC(=O)[C@H]3C[C@H]3C)ncc12 ; CNc1ncc(-c2nc3ccc(OC)cn3n2)c2cc(NC(=O)C3CC3)ncc12]

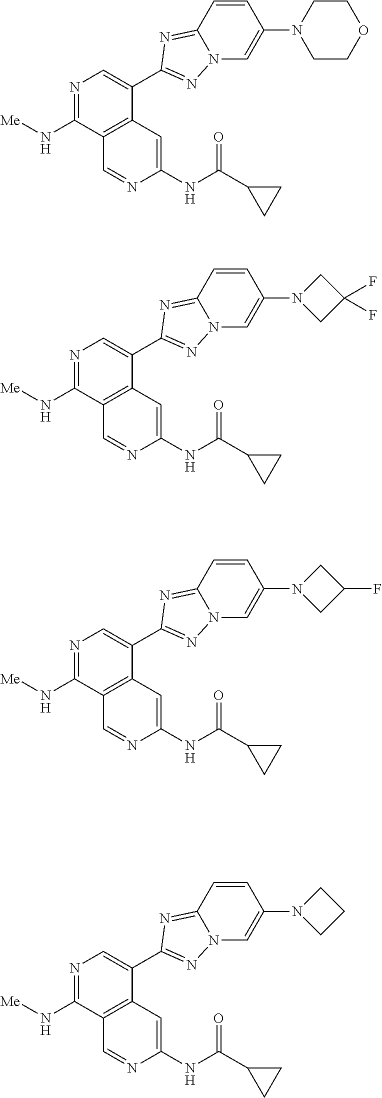 [SMILES: CNc1ncc(-c2nc3ccc(N4CCOCC4)cn3n2)c2cc(NC(=O)C3CC3)ncc12 ; CNc1ncc(-c2nc3ccc(N4CC(F)(F)C4)cn3n2)c2cc(NC(=O)C3CC3)ncc12 ; CNc1ncc(-c2nc3ccc(N4CC(F)C4)cn3n2)c2cc(NC(=O)C3CC3)ncc12 ; CNc1ncc(-c2nc3ccc(N4CCC4)cn3n2)c2cc(NC(=O)C3CC3)ncc12]

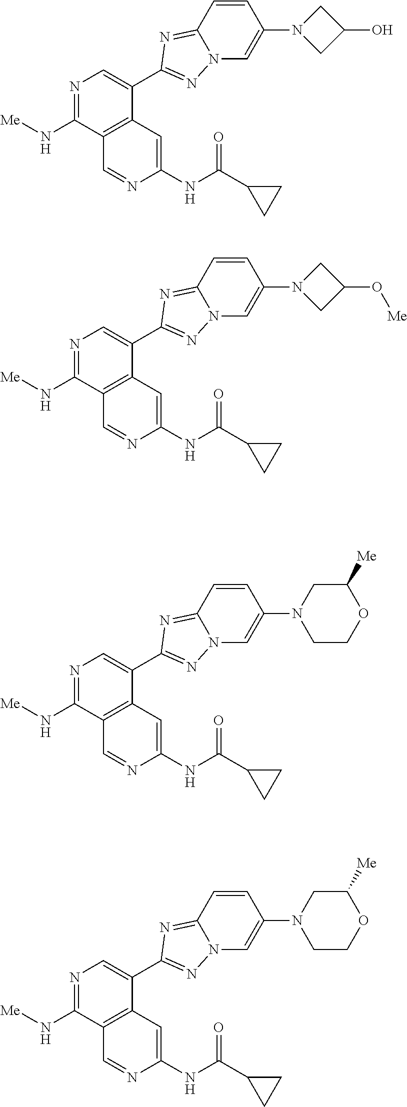

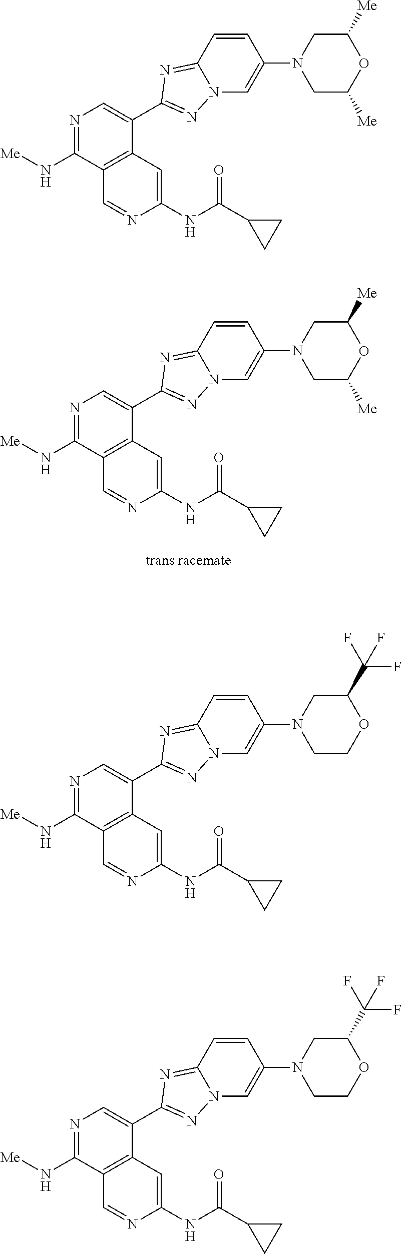 [SMILES: CNc1ncc(-c2nc3ccc(N4C[C@@H](C)O[C@@H](C)C4)cn3n2)c2cc(NC(=O)C3CC3)ncc12 ; CNc1ncc(-c2nc3ccc(N4C[C@@H](C)O[C@H](C)C4)cn3n2)c2cc(NC(=O)C3CC3)ncc12 ; CNc1ncc(-c2nc3ccc(N4CCO[C@H](C(F)(F)F)C4)cn3n2)c2cc(NC(=O)C3CC3)ncc12 ; CNc1ncc(-c2nc3ccc(N4CCO[C@@H](C(F)(F)F)C4)cn3n2)c2cc(NC(=O)C3CC3)ncc12]

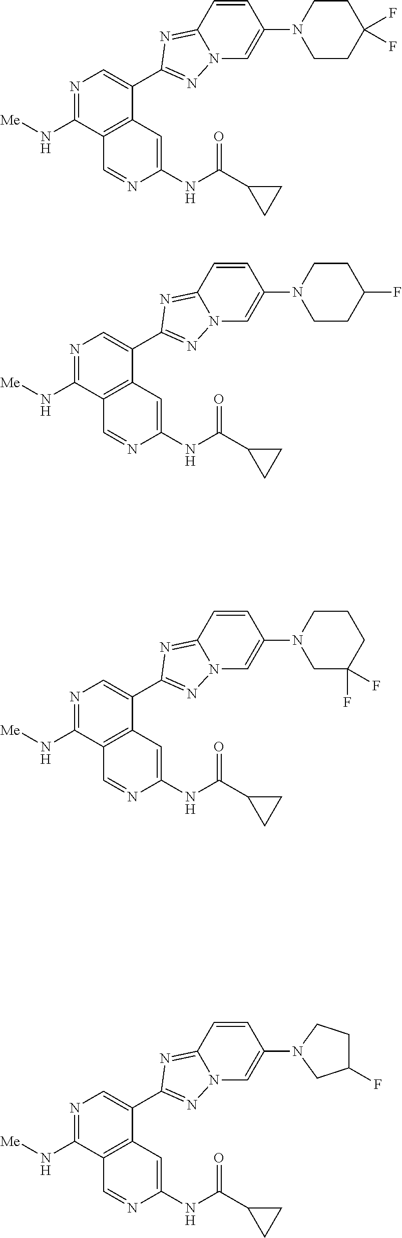 [SMILES: CNc1ncc(-c2nc3ccc(N4CCC(F)(F)CC4)cn3n2)c2cc(NC(=O)C3CC3)ncc12 ; CNc1ncc(-c2nc3ccc(N4CCC(F)CC4)cn3n2)c2cc(NC(=O)C3CC3)ncc12 ; CNc1ncc(-c2nc3ccc(N4CCCC(F)(F)C4)cn3n2)c2cc(NC(=O)C3CC3)ncc12 ; CNc1ncc(-c2nc3ccc(N4CCC(F)C4)cn3n2)c2cc(NC(=O)C3CC3)ncc12]

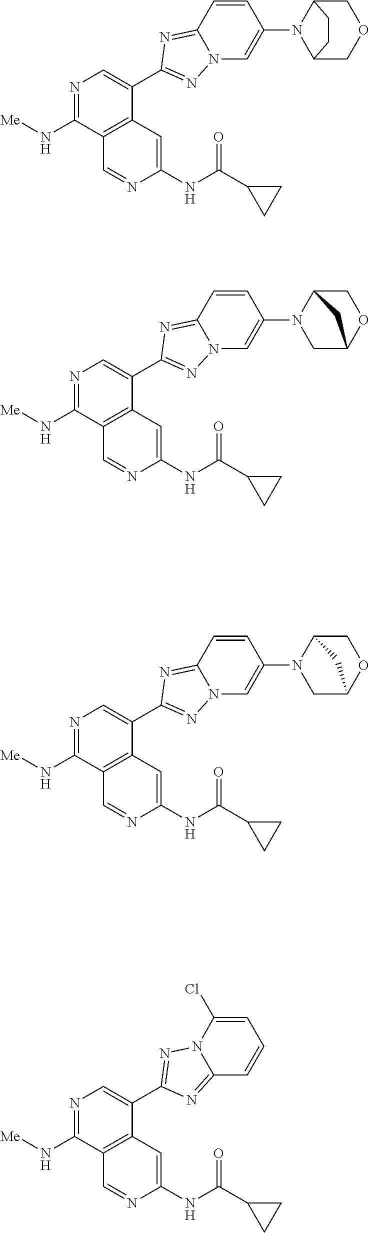 [SMILES: CNc1ncc(-c2nc3ccc(N4C5CCC4COC5)cn3n2)c2cc(NC(=O)C3CC3)ncc12 ; CNc1ncc(-c2nc3ccc(N4C[C@@H]5C[C@H]4CO5)cn3n2)c2cc(NC(=O)C3CC3)ncc12 ; CNc1ncc(-c2nc3ccc(N4C[C@H]5C[C@@H]4CO5)cn3n2)c2cc(NC(=O)C3CC3)ncc12 ; CNc1ncc(-c2nc3cccc(Cl)n3n2)c2cc(NC(=O)C3CC3)ncc12]

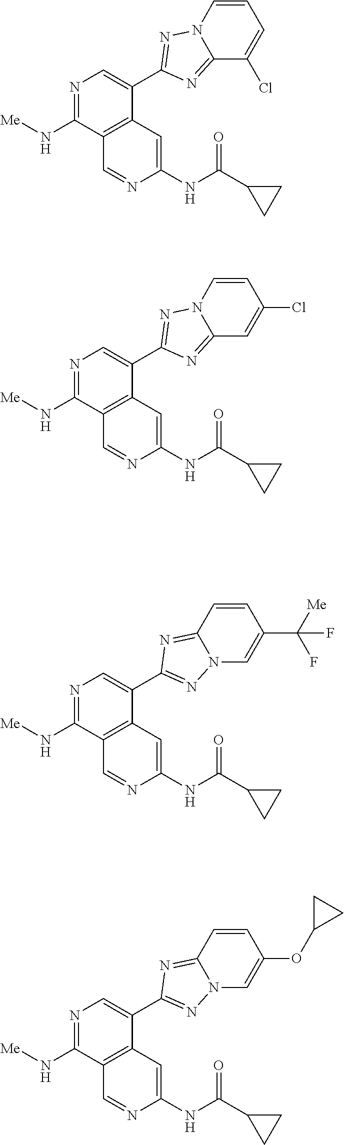 [SMILES: CNc1ncc(-c2nc3c(Cl)cccn3n2)c2cc(NC(=O)C3CC3)ncc12 ; CNc1ncc(-c2nc3cc(Cl)ccn3n2)c2cc(NC(=O)C3CC3)ncc12 ; CNc1ncc(-c2nc3ccc(C(C)(F)F)cn3n2)c2cc(NC(=O)C3CC3)ncc12 ; CNc1ncc(-c2nc3ccc(OC4CC4)cn3n2)c2cc(NC(=O)C3CC3)ncc12]

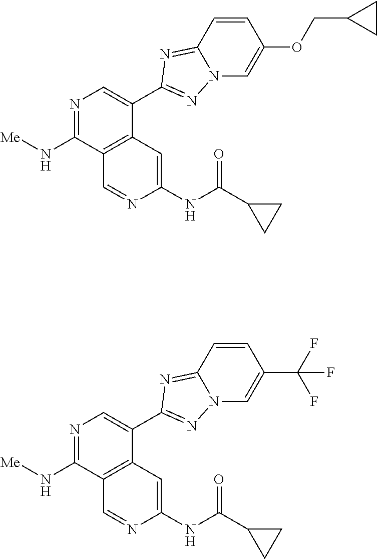 [SMILES: CNc1ncc(-c2nc3ccc(OCC4CC4)cn3n2)c2cc(NC(=O)C3CC3)ncc12 ; CNc1ncc(-c2nc3ccc(C(F)(F)F)cn3n2)c2cc(NC(=O)C3CC3)ncc12]

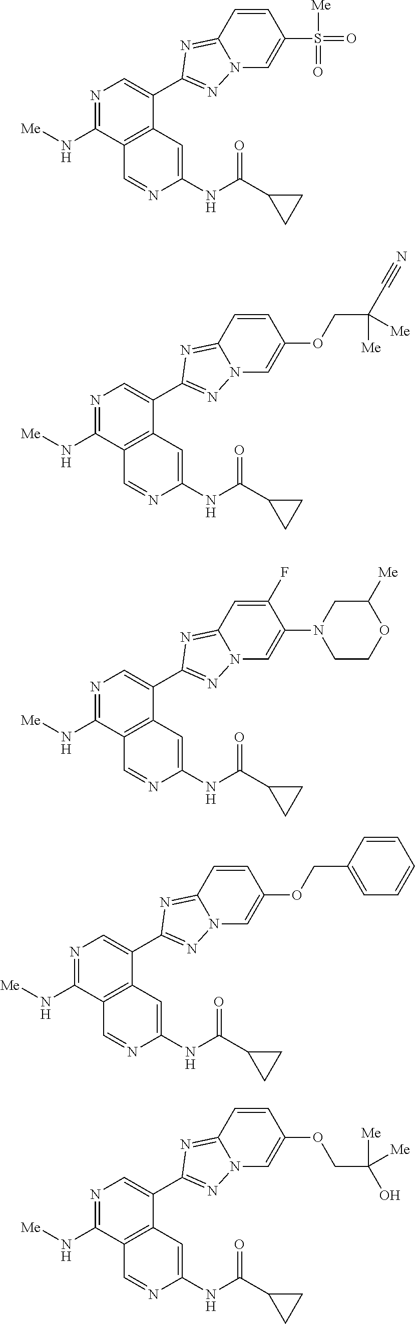

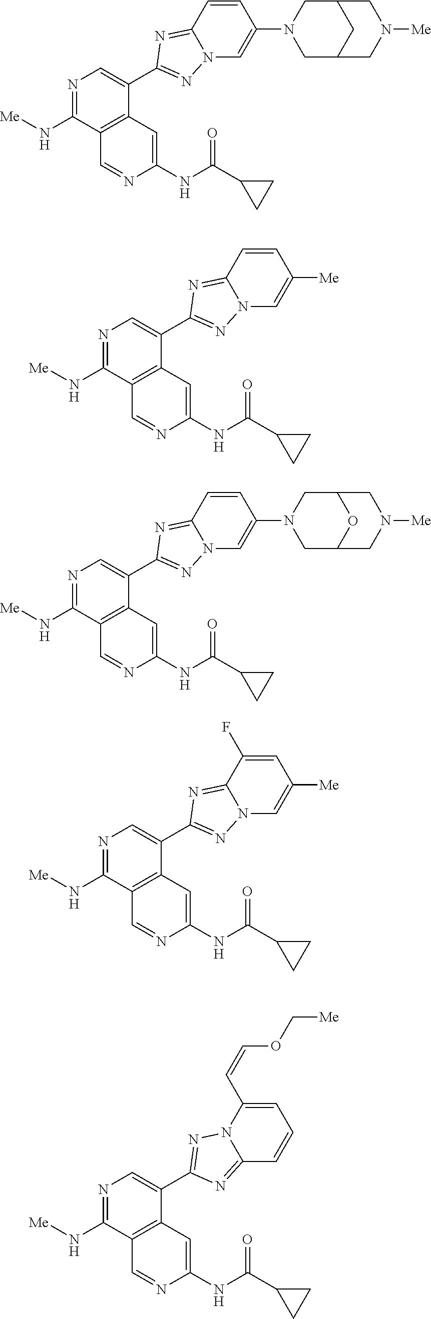

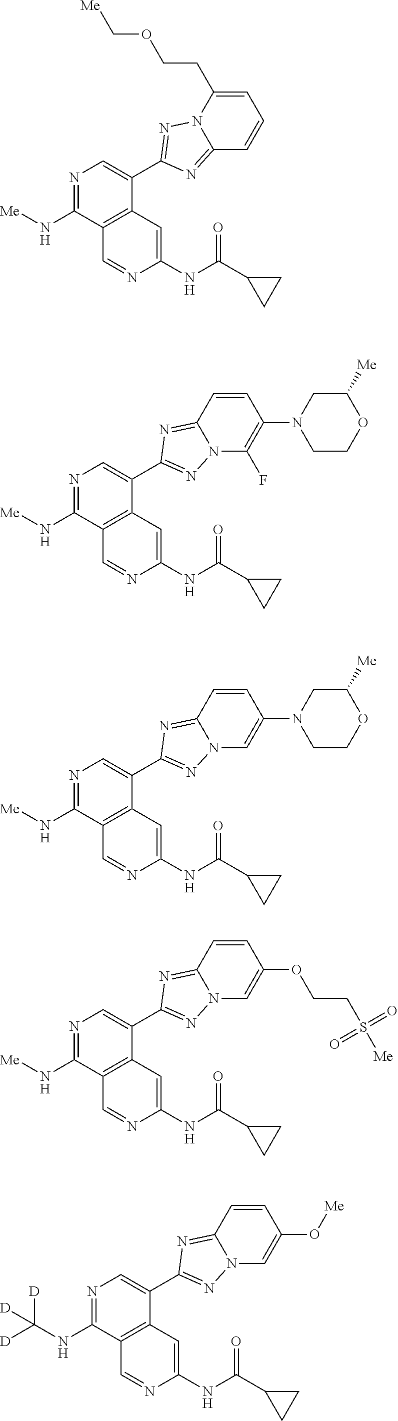

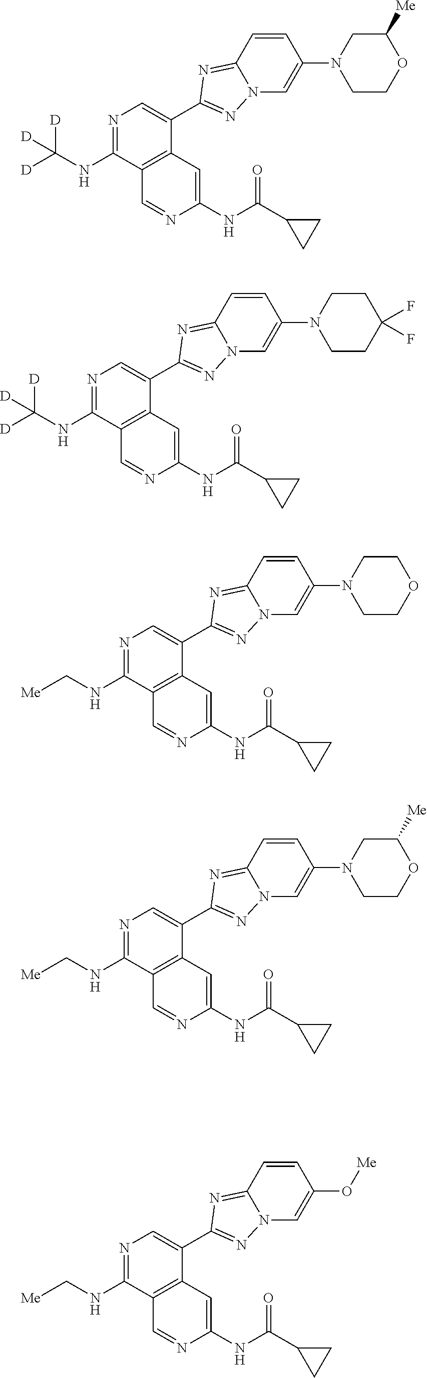 [SMILES: [2H]C([2H])([2H])Nc1ncc(-c2nc3ccc(N4CCO[C@H](C)C4)cn3n2)c2cc(NC(=O)C3CC3)ncc12 ; [2H]C([2H])([2H])Nc1ncc(-c2nc3ccc(N4CCC(F)(F)CC4)cn3n2)c2cc(NC(=O)C3CC3)ncc12 ; CCNc1ncc(-c2nc3ccc(N4CCOCC4)cn3n2)c2cc(NC(=O)C3CC3)ncc12 ; CCNc1ncc(-c2nc3ccc(N4CCO[C@@H](C)C4)cn3n2)c2cc(NC(=O)C3CC3)ncc12 ; CCNc1ncc(-c2nc3ccc(OC)cn3n2)c2cc(NC(=O)C3CC3)ncc12]

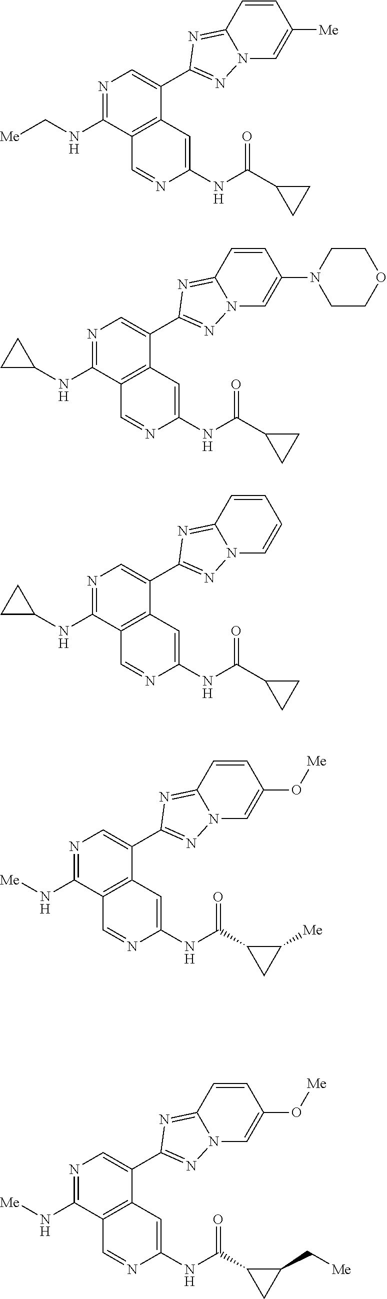

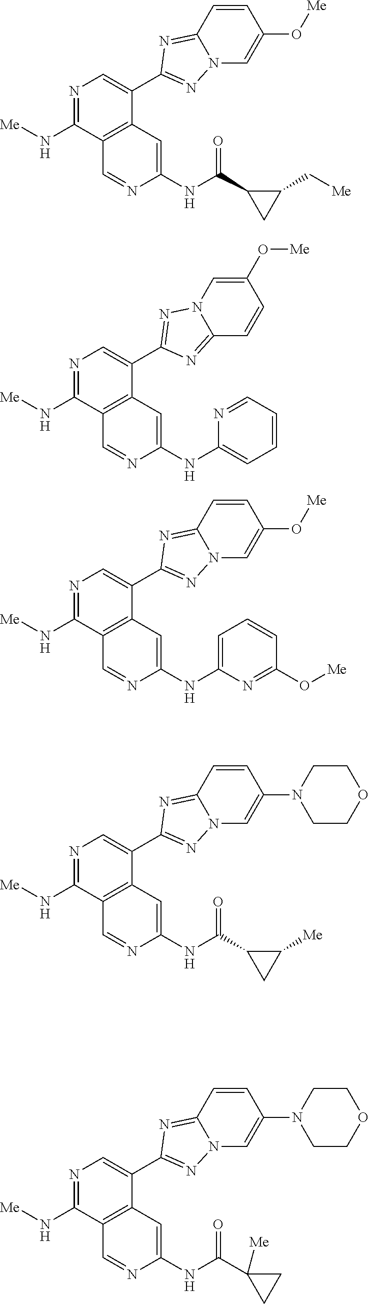

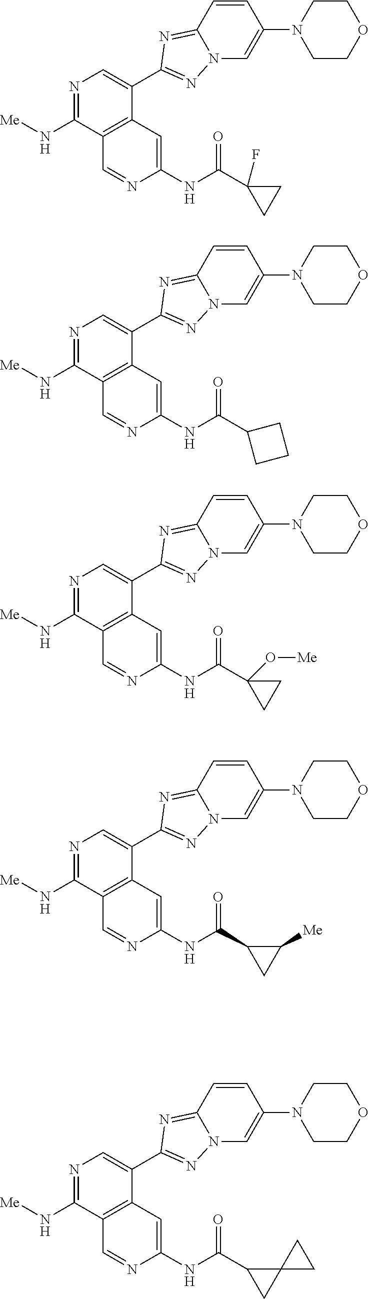

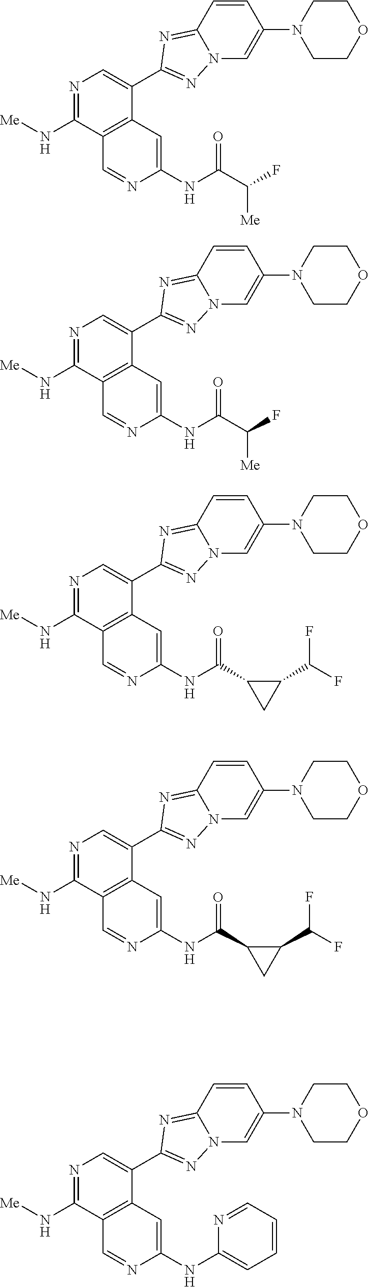

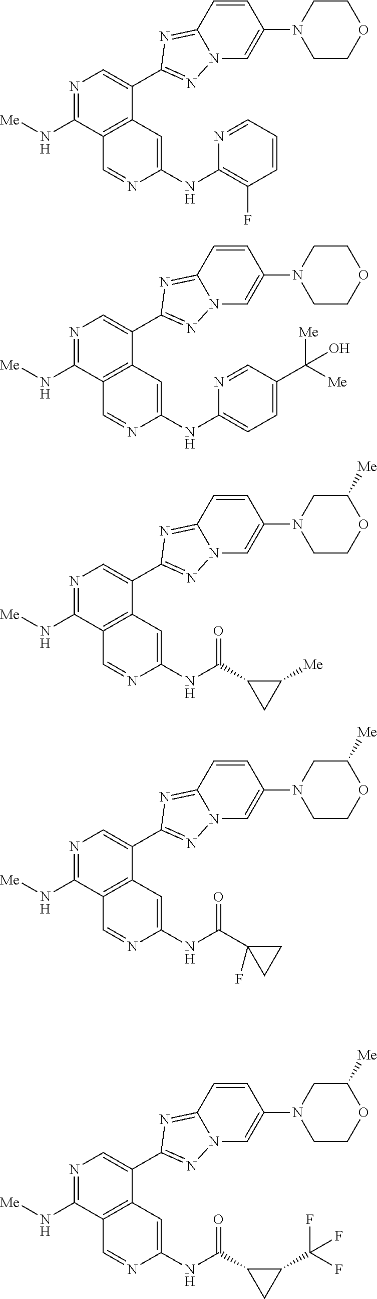

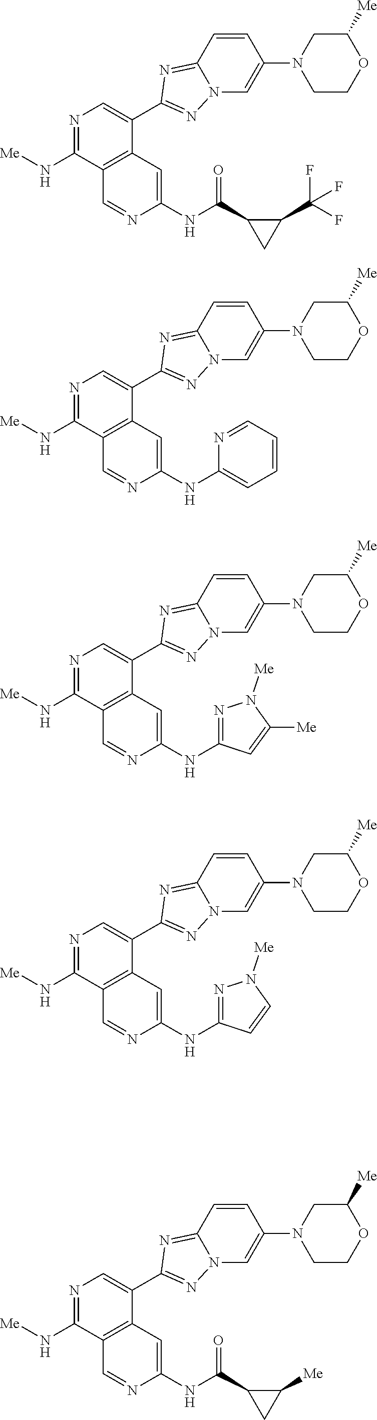 [SMILES: CNc1ncc(-c2nc3ccc(N4CCO[C@@H](C)C4)cn3n2)c2cc(NC(=O)[C@@H]3C[C@@H]3C(F)(F)F)ncc12 ; CNc1ncc(-c2nc3ccc(N4CCO[C@@H](C)C4)cn3n2)c2cc(Nc3ccccn3)ncc12 ; CNc1ncc(-c2nc3ccc(N4CCO[C@@H](C)C4)cn3n2)c2cc(Nc3cc(C)n(C)n3)ncc12 ; CNc1ncc(-c2nc3ccc(N4CCO[C@@H](C)C4)cn3n2)c2cc(Nc3ccn(C)n3)ncc12 ; CNc1ncc(-c2nc3ccc(N4CCO[C@H](C)C4)cn3n2)c2cc(NC(=O)[C@@H]3C[C@@H]3C)ncc12]

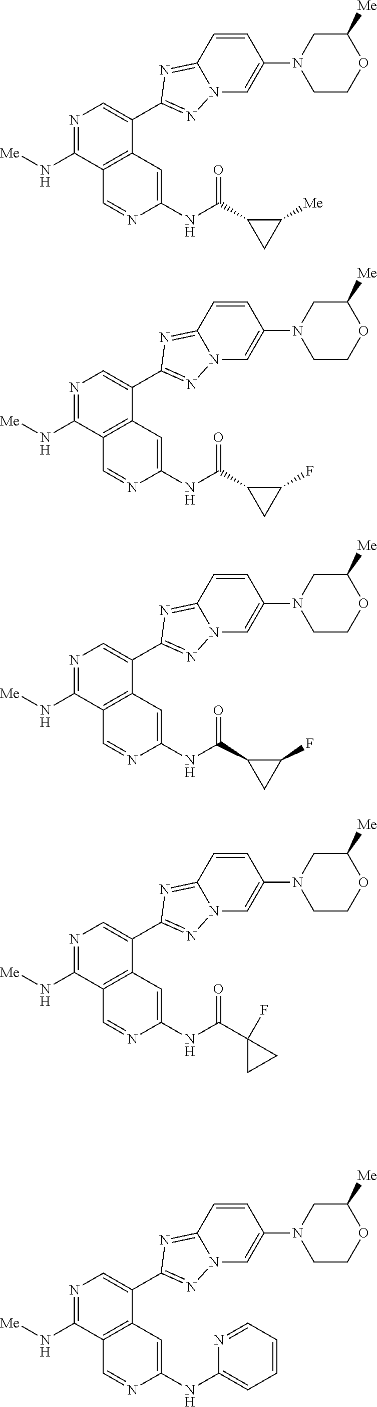 [SMILES: CNc1ncc(-c2nc3ccc(N4CCO[C@H](C)C4)cn3n2)c2cc(NC(=O)[C@H]3C[C@H]3C)ncc12 ; CNc1ncc(-c2nc3ccc(N4CCO[C@H](C)C4)cn3n2)c2cc(NC(=O)[C@H]3C[C@H]3F)ncc12 ; CNc1ncc(-c2nc3ccc(N4CCO[C@H](C)C4)cn3n2)c2cc(NC(=O)[C@@H]3C[C@@H]3F)ncc12 ; CNc1ncc(-c2nc3ccc(N4CCO[C@H](C)C4)cn3n2)c2cc(NC(=O)C3(F)CC3)ncc12 ; CNc1ncc(-c2nc3ccc(N4CCO[C@H](C)C4)cn3n2)c2cc(Nc3ccccn3)ncc12]

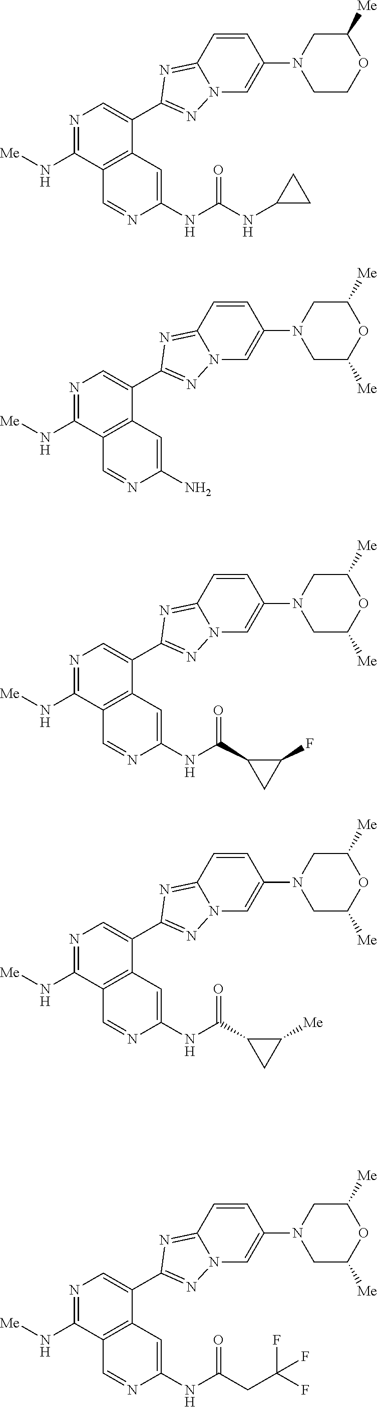 [SMILES: CNc1ncc(-c2nc3ccc(N4CCO[C@H](C)C4)cn3n2)c2cc(NC(=O)NC3CC3)ncc12 ; CNc1ncc(-c2nc3ccc(N4C[C@@H](C)O[C@@H](C)C4)cn3n2)c2cc(N)ncc12 ; CNc1ncc(-c2nc3ccc(N4C[C@@H](C)O[C@@H](C)C4)cn3n2)c2cc(NC(=O)[C@@H]3C[C@@H]3F)ncc12 ; CNc1ncc(-c2nc3ccc(N4C[C@@H](C)O[C@@H](C)C4)cn3n2)c2cc(NC(=O)[C@H]3C[C@H]3C)ncc12 ; CNc1ncc(-c2nc3ccc(N4C[C@@H](C)O[C@@H](C)C4)cn3n2)c2cc(NC(=O)CC(F)(F)F)ncc12]

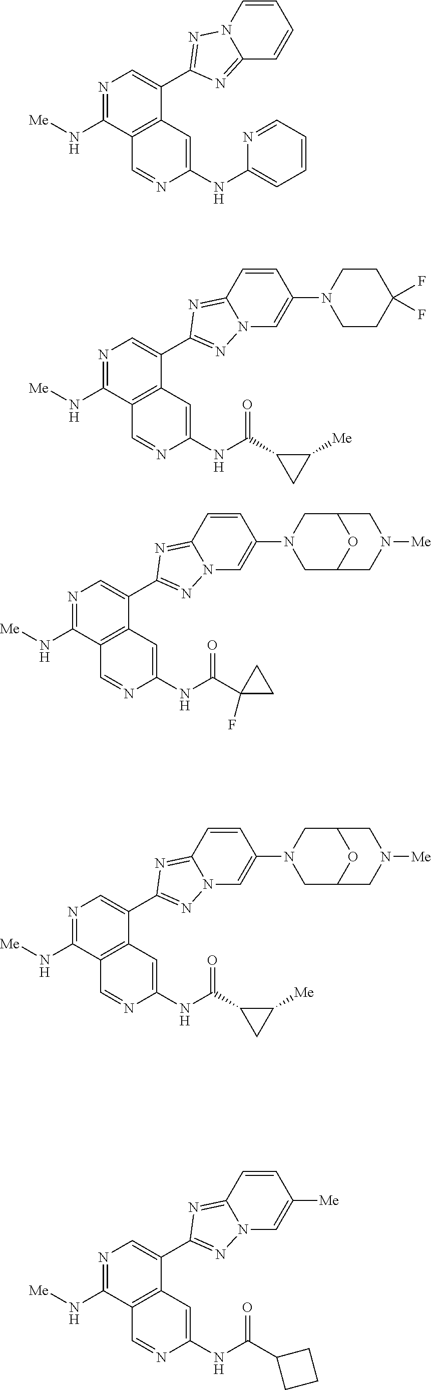

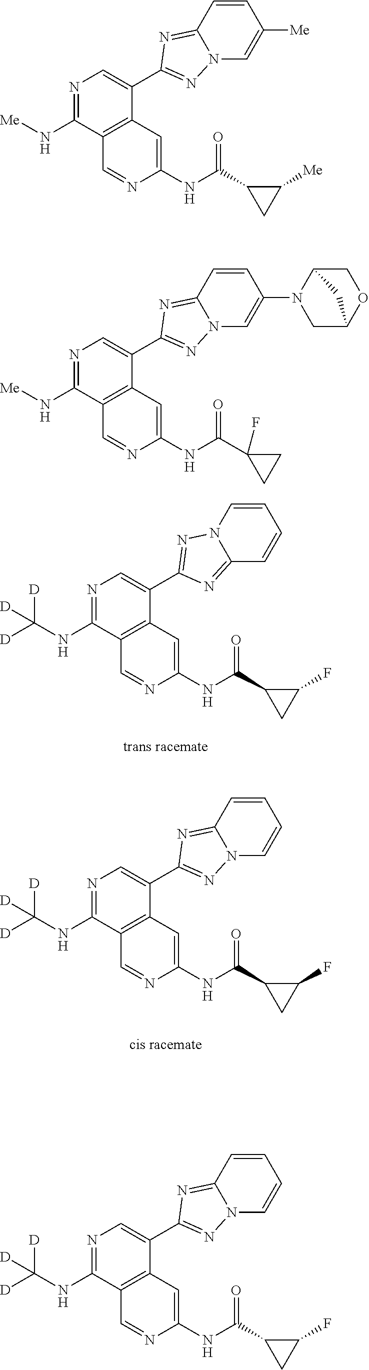

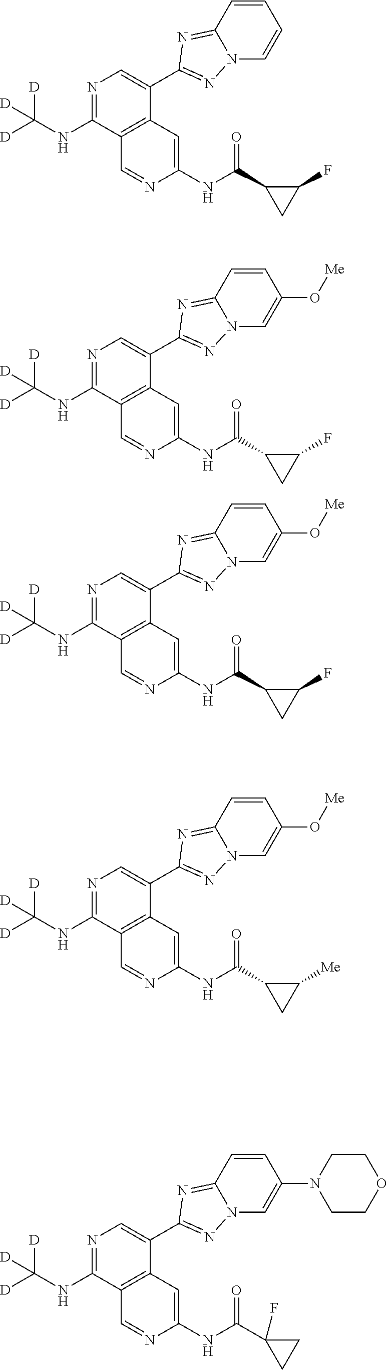

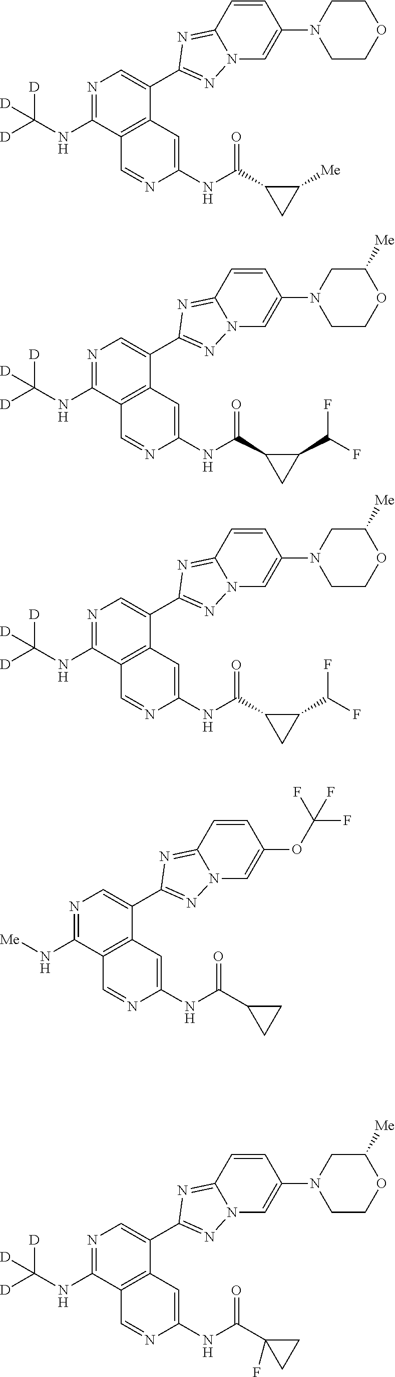 [SMILES: [2H]C([2H])([2H])Nc1ncc(-c2nc3ccc(N4CCOCC4)cn3n2)c2cc(NC(=O)[C@H]3C[C@H]3C)ncc12 ; [2H]C([2H])([2H])Nc1ncc(-c2nc3ccc(N4CCO[C@@H](C)C4)cn3n2)c2cc(NC(=O)[C@@H]3C[C@@H]3C(F)F)ncc12 ; [2H]C([2H])([2H])Nc1ncc(-c2nc3ccc(N4CCO[C@@H](C)C4)cn3n2)c2cc(NC(=O)[C@H]3C[C@H]3C(F)F)ncc12 ; CNc1ncc(-c2nc3ccc(OC(F)(F)F)cn3n2)c2cc(NC(=O)C3CC3)ncc12 ; [2H]C([2H])([2H])Nc1ncc(-c2nc3ccc(N4CCO[C@@H](C)C4)cn3n2)c2cc(NC(=O)C3(F)CC3)ncc12]

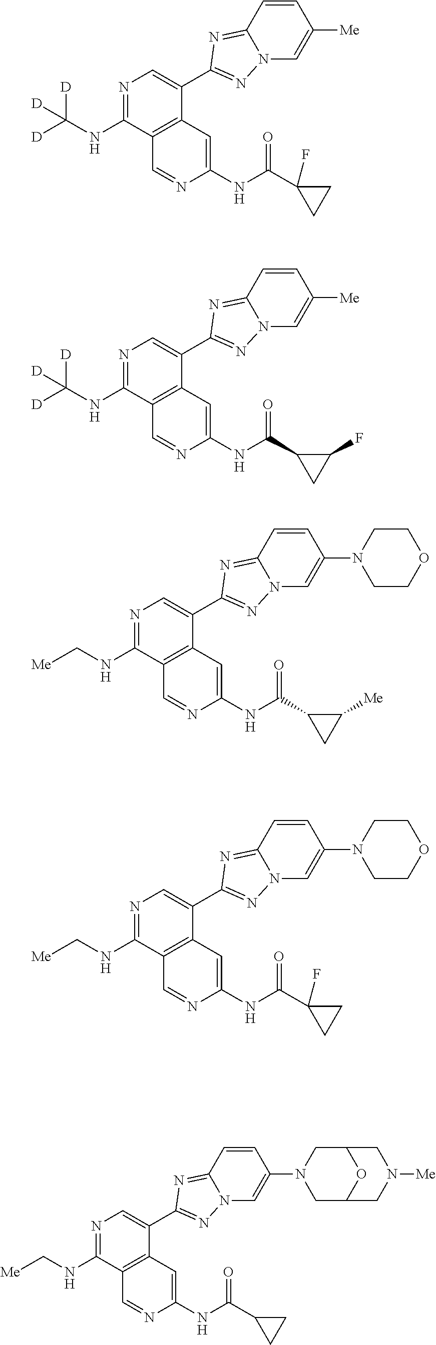

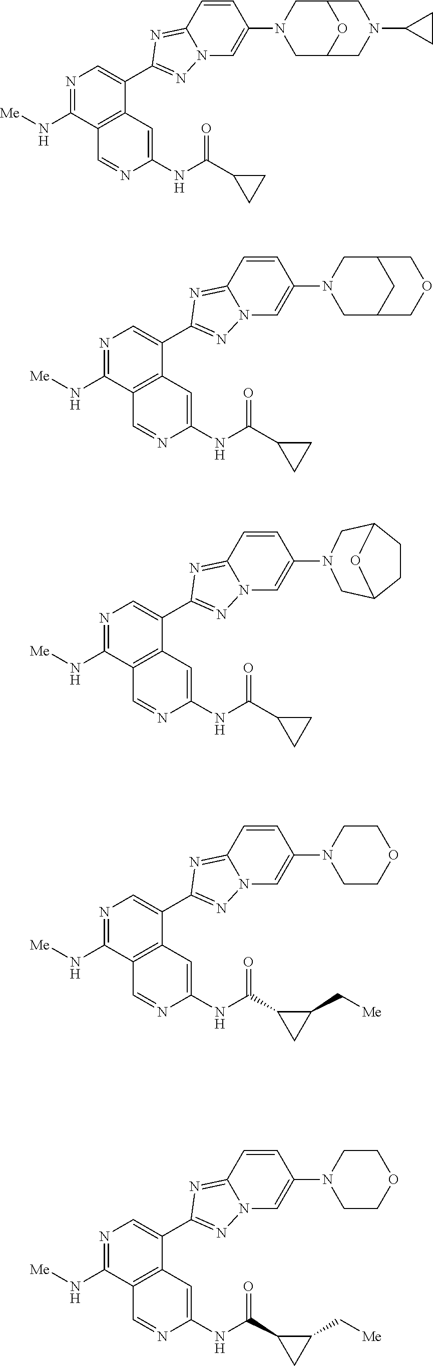 [SMILES: CNc1ncc(-c2nc3ccc(N4CC5CN(C6CC6)CC(C4)O5)cn3n2)c2cc(NC(=O)C3CC3)ncc12 ; CNc1ncc(-c2nc3ccc(N4CC5COCC(C5)C4)cn3n2)c2cc(NC(=O)C3CC3)ncc12 ; CNc1ncc(-c2nc3ccc(N4CC5CCC(C4)O5)cn3n2)c2cc(NC(=O)C3CC3)ncc12 ; CC[C@H]1C[C@@H]1C(=O)Nc1cc2c(-c3nc4ccc(N5CCOCC5)cn4n3)cnc(NC)c2cn1 ; CC[C@@H]1C[C@H]1C(=O)Nc1cc2c(-c3nc4ccc(N5CCOCC5)cn4n3)cnc(NC)c2cn1]

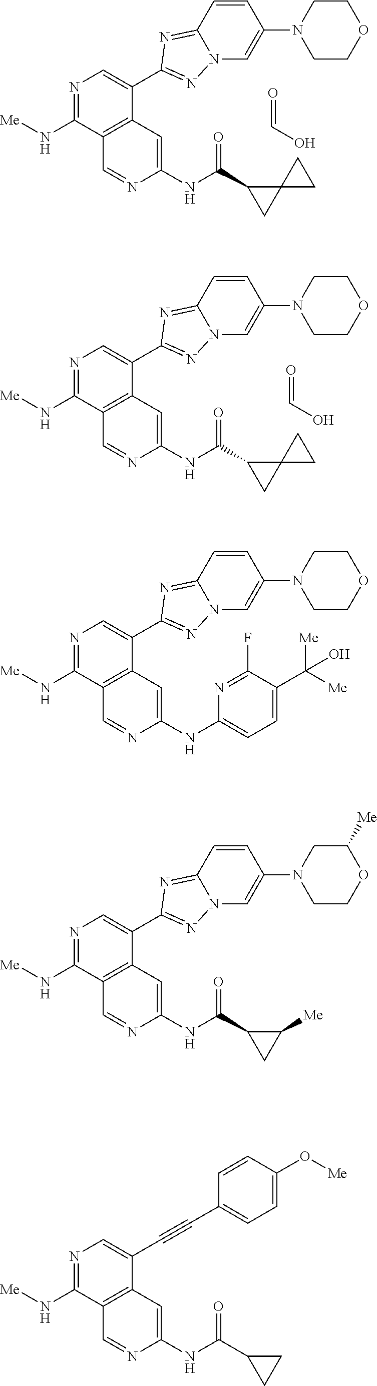

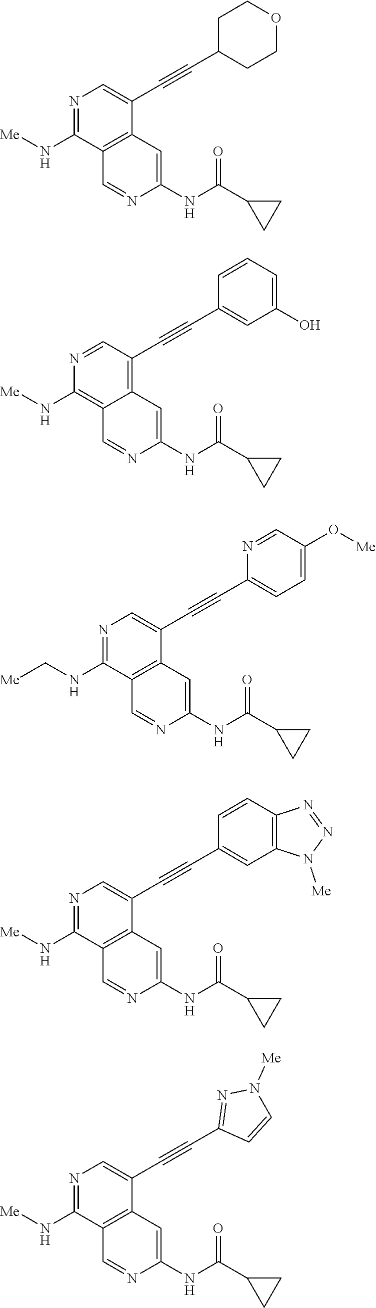

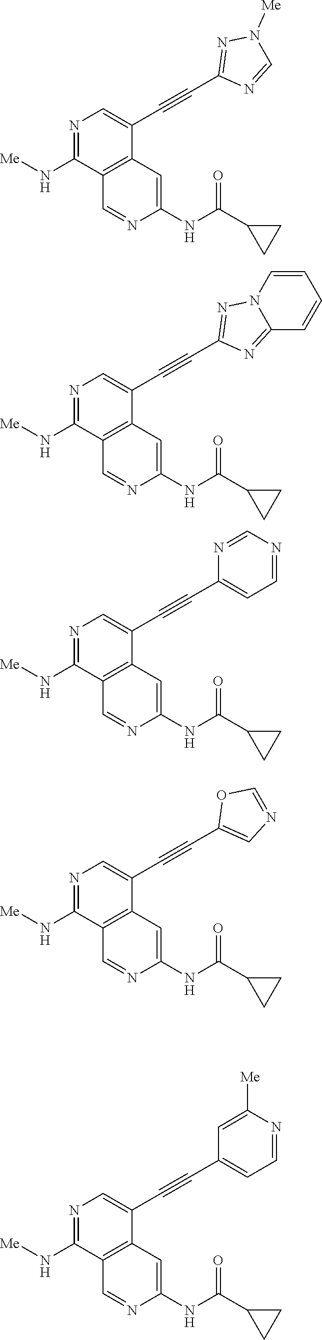

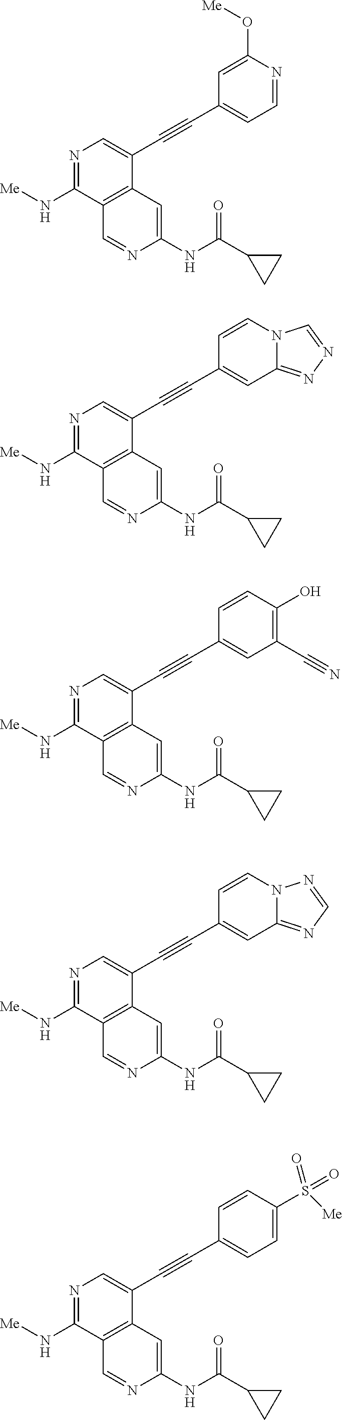

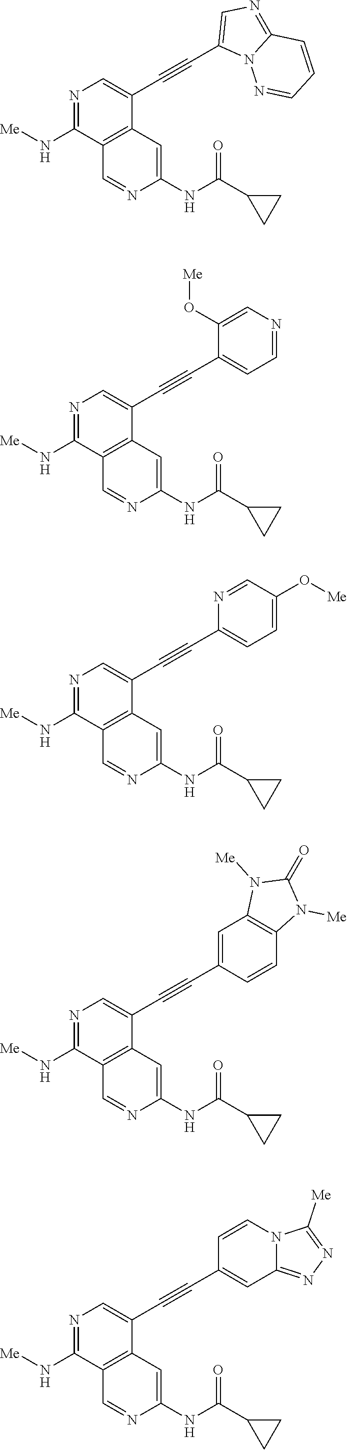 [SMILES: CNc1ncc(C#Cc2cnc3cccnn23)c2cc(NC(=O)C3CC3)ncc12 ; CNc1ncc(C#Cc2ccncc2OC)c2cc(NC(=O)C3CC3)ncc12 ; CNc1ncc(C#Cc2ccc(OC)cn2)c2cc(NC(=O)C3CC3)ncc12 ; CNc1ncc(C#Cc2ccc3c(c2)n(C)c(=O)n3C)c2cc(NC(=O)C3CC3)ncc12 ; CNc1ncc(C#Cc2ccn3c(C)nnc3c2)c2cc(NC(=O)C3CC3)ncc12]

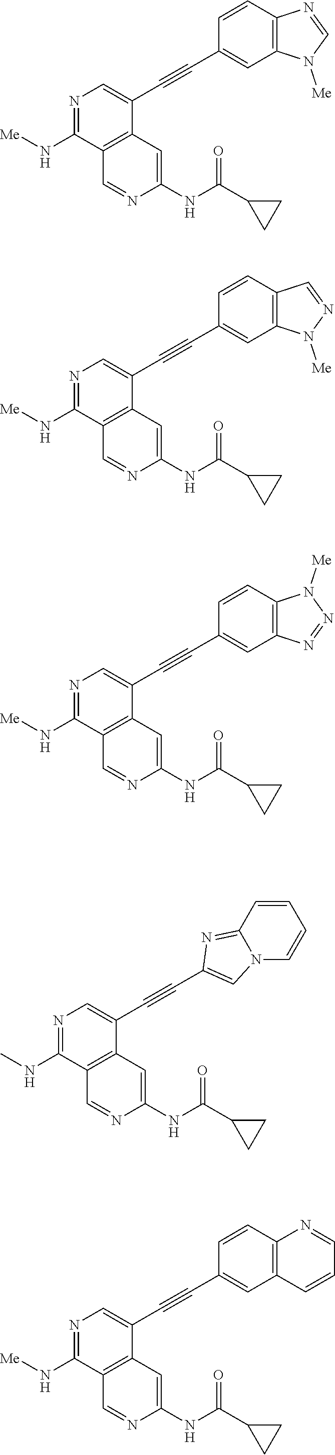 [SMILES: CNc1ncc(C#Cc2ccc3ncn(C)c3c2)c2cc(NC(=O)C3CC3)ncc12 ; CNc1ncc(C#Cc2ccc3cnn(C)c3c2)c2cc(NC(=O)C3CC3)ncc12 ; CNc1ncc(C#Cc2ccc3c(c2)nnn3C)c2cc(NC(=O)C3CC3)ncc12 ; CNc1ncc(C#Cc2cn3ccccc3n2)c2cc(NC(=O)C3CC3)ncc12 ; CNc1ncc(C#Cc2ccc3ncccc3c2)c2cc(NC(=O)C3CC3)ncc12]

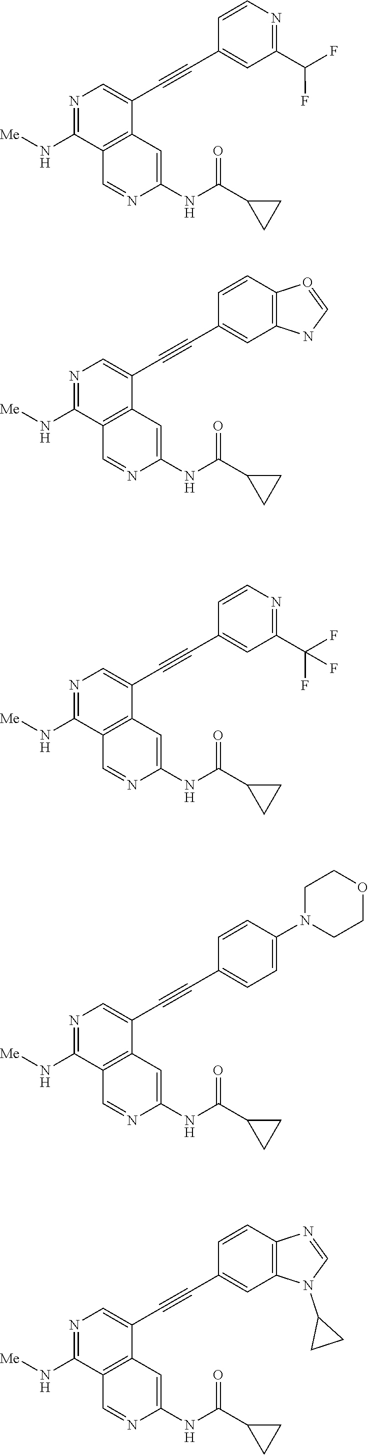

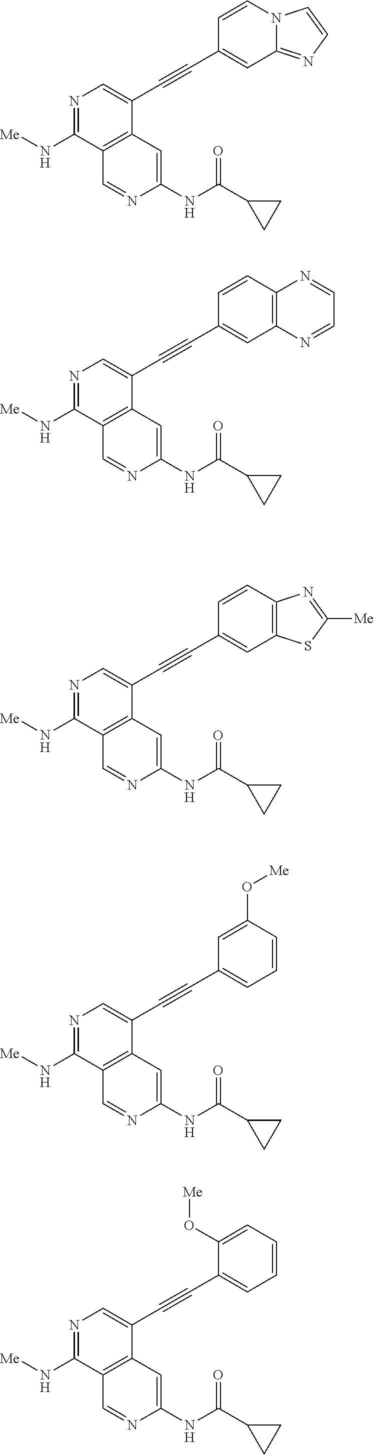 [SMILES: CNc1ncc(C#Cc2ccn3ccnc3c2)c2cc(NC(=O)C3CC3)ncc12 ; CNc1ncc(C#Cc2ccc3nccnc3c2)c2cc(NC(=O)C3CC3)ncc12 ; CNc1ncc(C#Cc2ccc3nc(C)sc3c2)c2cc(NC(=O)C3CC3)ncc12 ; CNc1ncc(C#Cc2cccc(OC)c2)c2cc(NC(=O)C3CC3)ncc12 ; CNc1ncc(C#Cc2ccccc2OC)c2cc(NC(=O)C3CC3)ncc12]

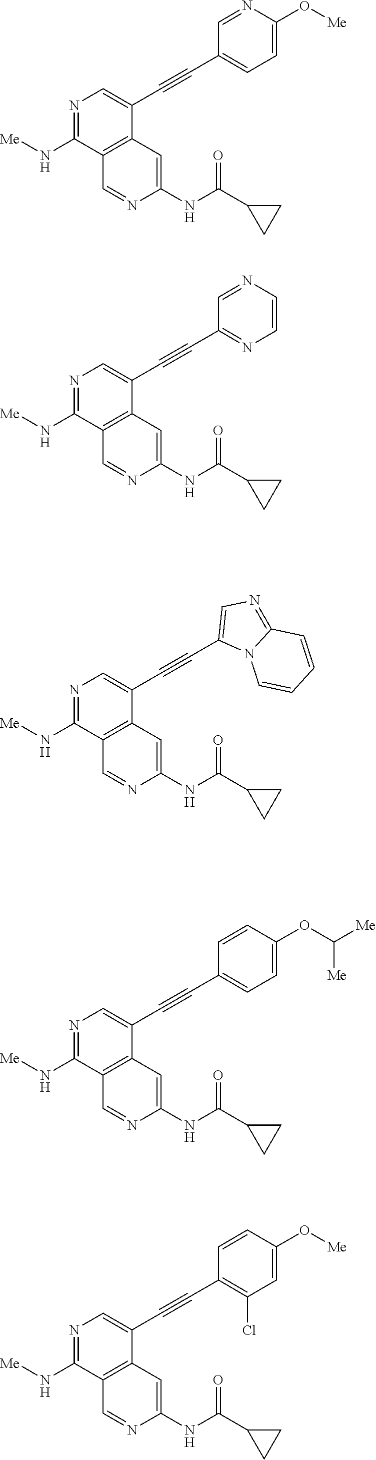 [SMILES: CNc1ncc(C#Cc2ccc(OC)nc2)c2cc(NC(=O)C3CC3)ncc12 ; CNc1ncc(C#Cc2cnccn2)c2cc(NC(=O)C3CC3)ncc12 ; CNc1ncc(C#Cc2cnc3ccccn23)c2cc(NC(=O)C3CC3)ncc12 ; CNc1ncc(C#Cc2ccc(OC(C)C)cc2)c2cc(NC(=O)C3CC3)ncc12 ; CNc1ncc(C#Cc2ccc(OC)cc2Cl)c2cc(NC(=O)C3CC3)ncc12]

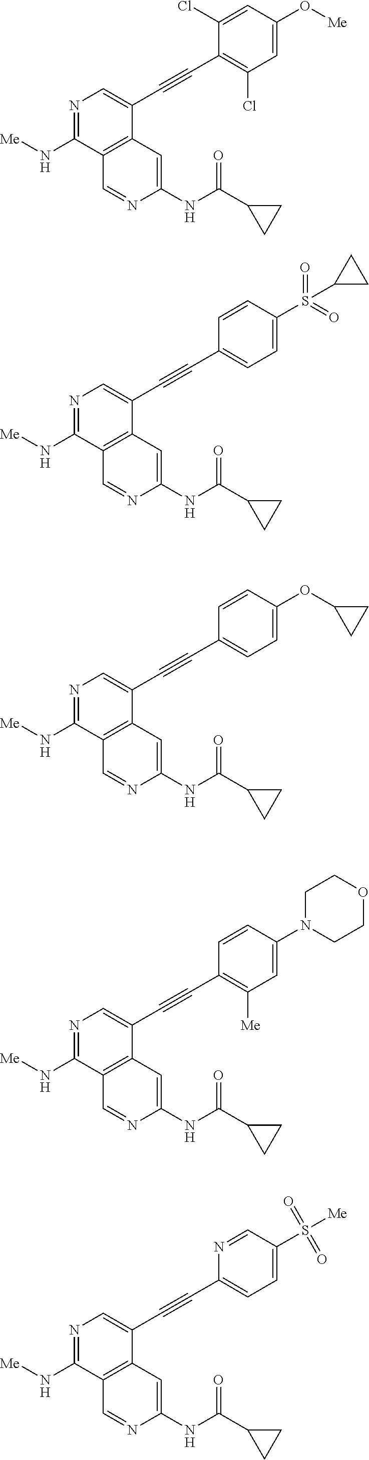 [SMILES: CNc1ncc(C#Cc2c(Cl)cc(OC)cc2Cl)c2cc(NC(=O)C3CC3)ncc12 ; CNc1ncc(C#Cc2ccc(S(=O)(=O)C3CC3)cc2)c2cc(NC(=O)C3CC3)ncc12 ; CNc1ncc(C#Cc2ccc(OC3CC3)cc2)c2cc(NC(=O)C3CC3)ncc12 ; CNc1ncc(C#Cc2ccc(N3CCOCC3)cc2C)c2cc(NC(=O)C3CC3)ncc12 ; CNc1ncc(C#Cc2ccc(S(C)(=O)=O)cn2)c2cc(NC(=O)C3CC3)ncc12]

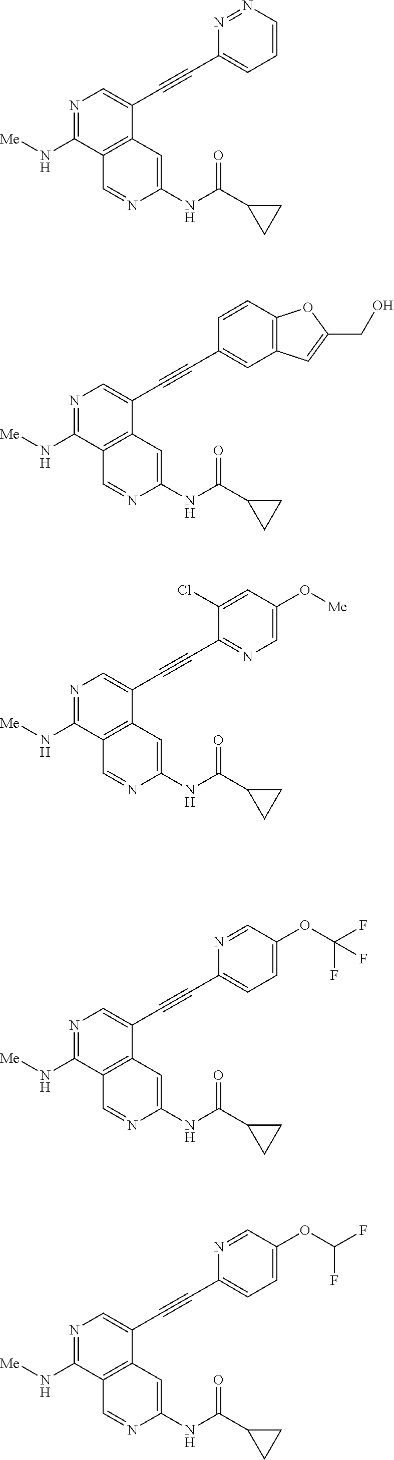 [SMILES: CNc1ncc(C#Cc2cccnn2)c2cc(NC(=O)C3CC3)ncc12 ; CNc1ncc(C#Cc2ccc3oc(CO)cc3c2)c2cc(NC(=O)C3CC3)ncc12 ; CNc1ncc(C#Cc2ncc(OC)cc2Cl)c2cc(NC(=O)C3CC3)ncc12 ; CNc1ncc(C#Cc2ccc(OC(F)(F)F)cn2)c2cc(NC(=O)C3CC3)ncc12 ; CNc1ncc(C#Cc2ccc(OC(F)F)cn2)c2cc(NC(=O)C3CC3)ncc12]

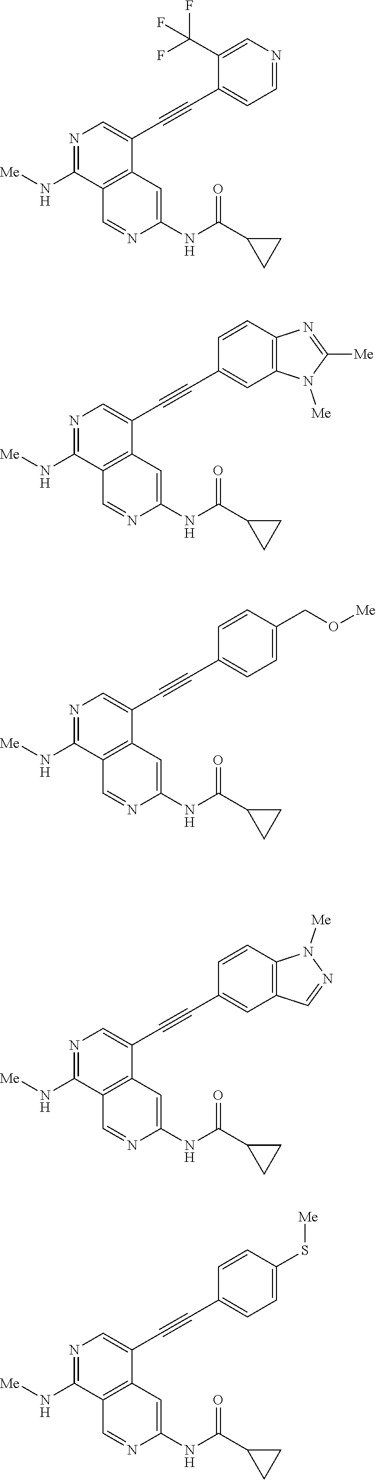 [SMILES: CNc1ncc(C#Cc2ccncc2C(F)(F)F)c2cc(NC(=O)C3CC3)ncc12 ; CNc1ncc(C#Cc2ccc3nc(C)n(C)c3c2)c2cc(NC(=O)C3CC3)ncc12 ; CNc1ncc(C#Cc2ccc(COC)cc2)c2cc(NC(=O)C3CC3)ncc12 ; CNc1ncc(C#Cc2ccc3c(cnn3C)c2)c2cc(NC(=O)C3CC3)ncc12 ; CNc1ncc(C#Cc2ccc(SC)cc2)c2cc(NC(=O)C3CC3)ncc12]

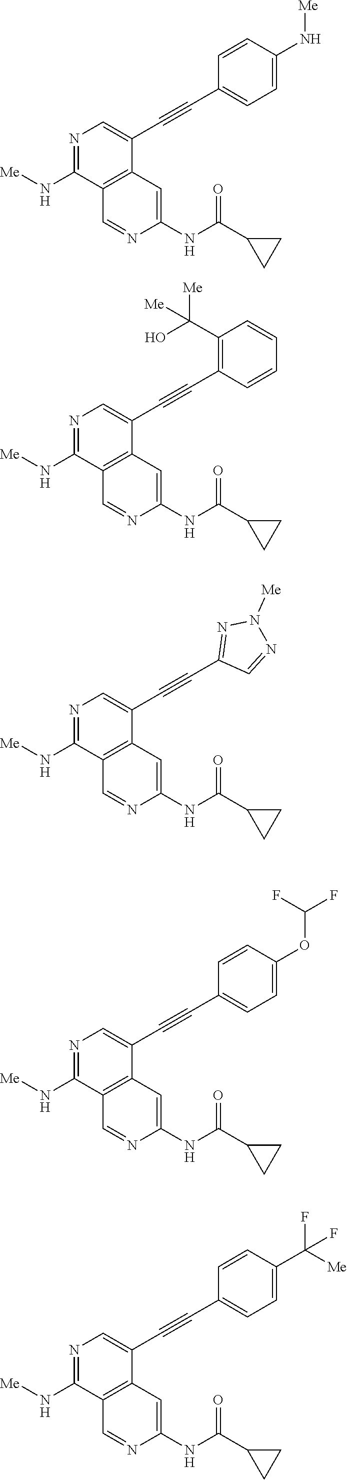

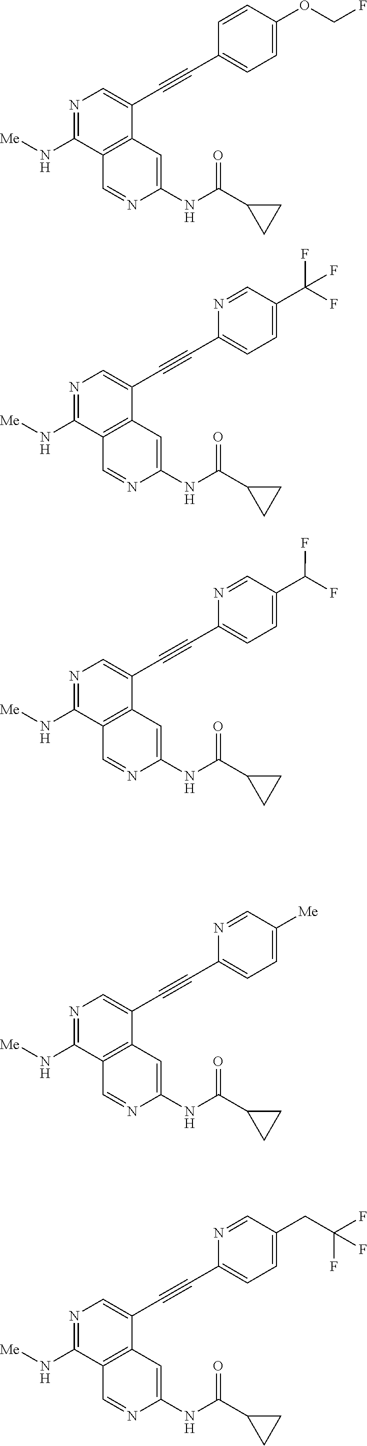

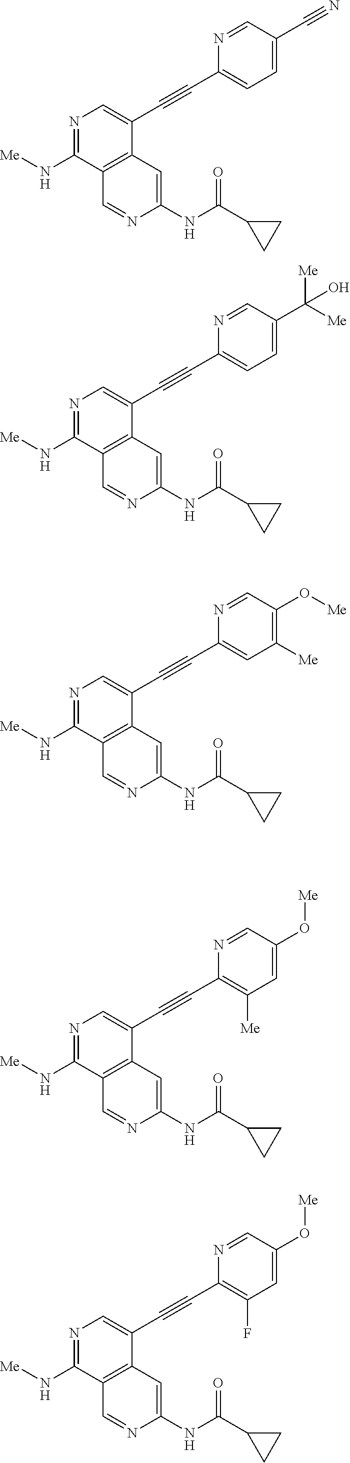

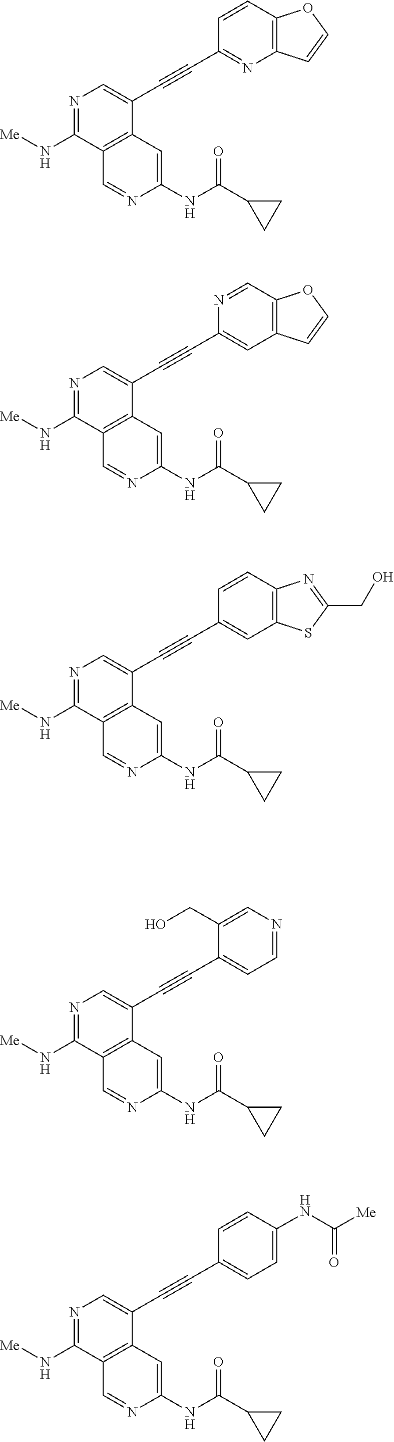 [SMILES: CNc1ncc(C#Cc2ccc3occc3n2)c2cc(NC(=O)C3CC3)ncc12 ; CNc1ncc(C#Cc2cc3ccoc3cn2)c2cc(NC(=O)C3CC3)ncc12 ; CNc1ncc(C#Cc2ccc3nc(CO)sc3c2)c2cc(NC(=O)C3CC3)ncc12 ; CNc1ncc(C#Cc2ccncc2CO)c2cc(NC(=O)C3CC3)ncc12 ; CNc1ncc(C#Cc2ccc(NC(C)=O)cc2)c2cc(NC(=O)C3CC3)ncc12]

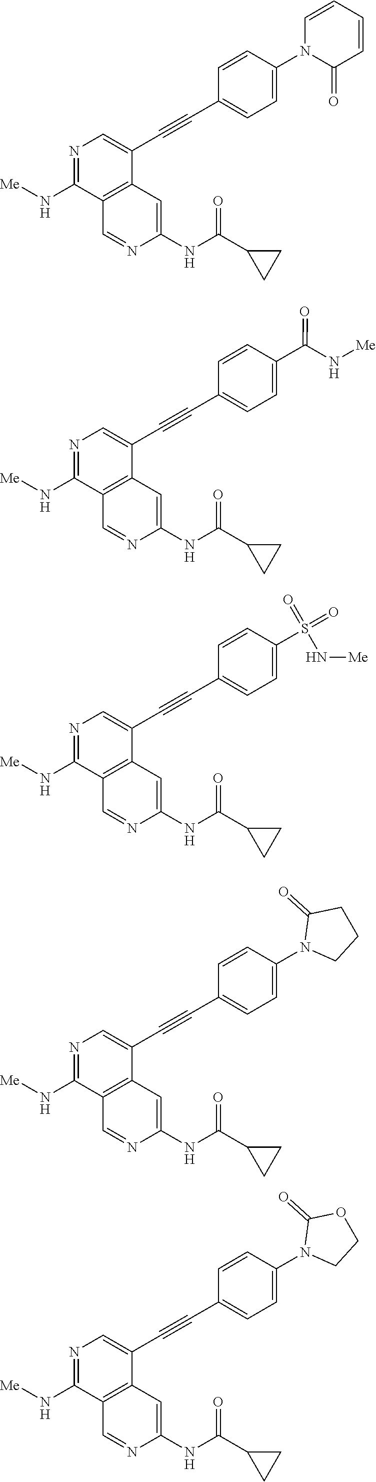

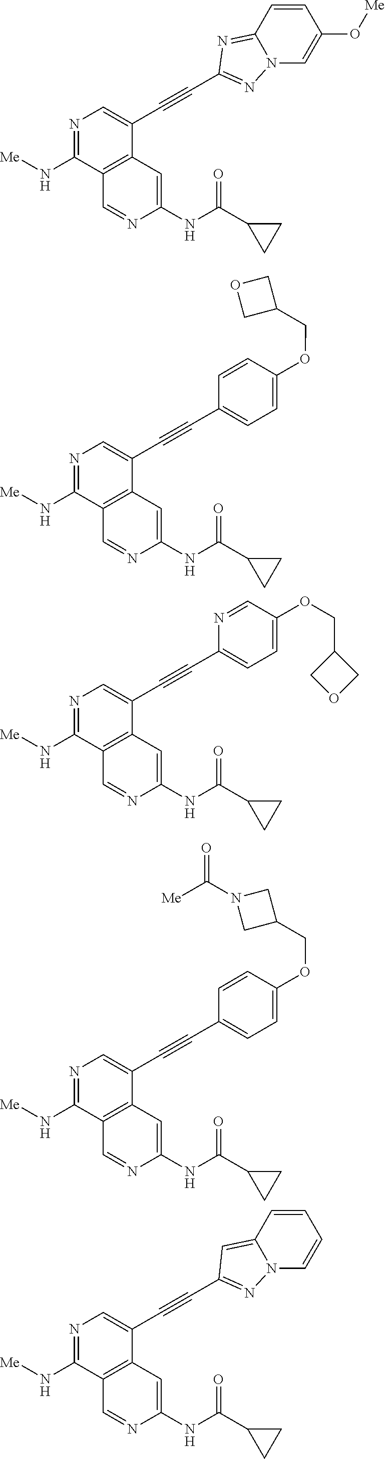

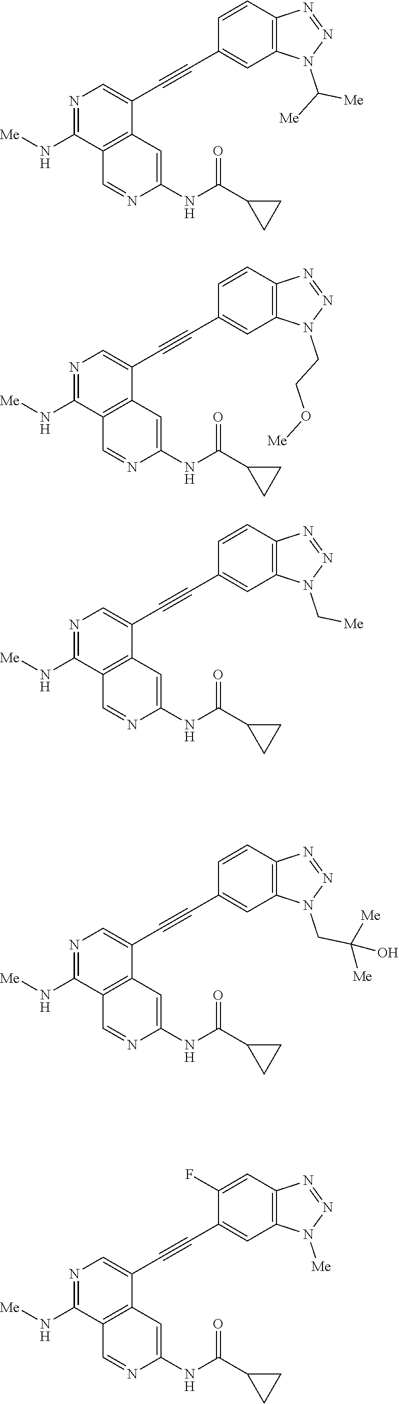

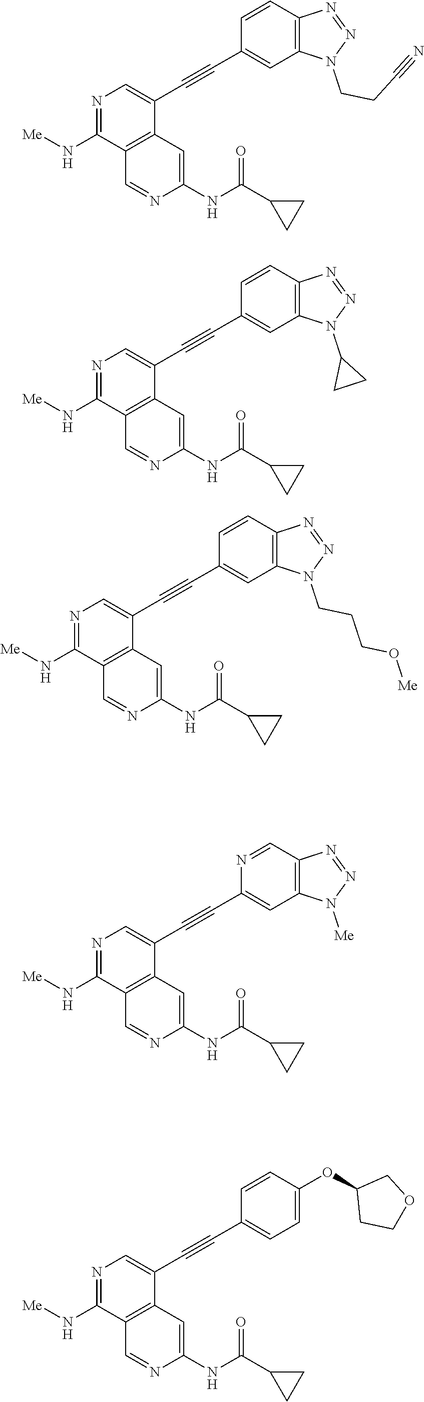

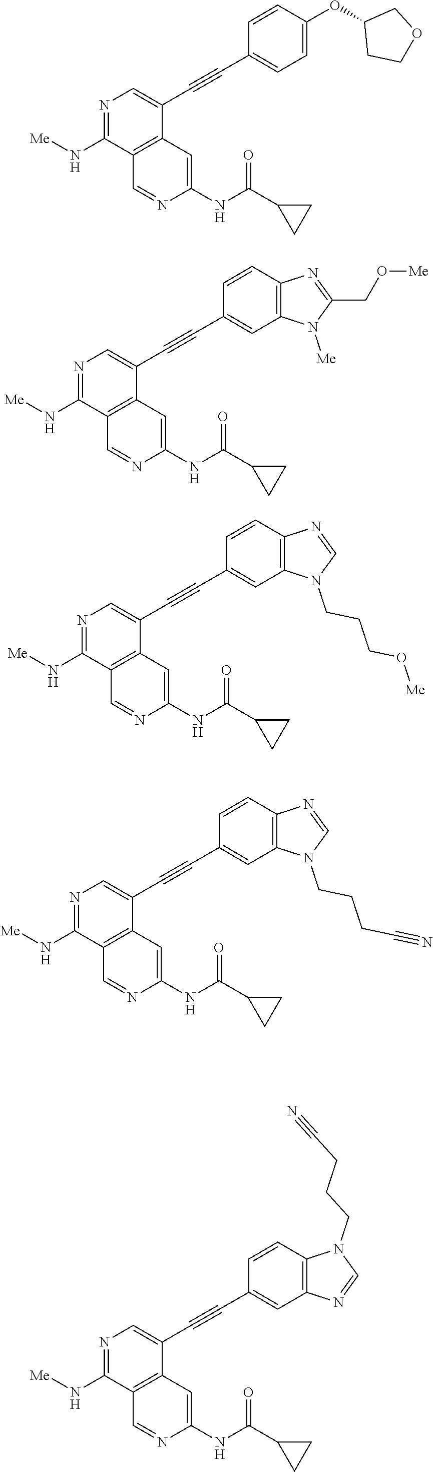

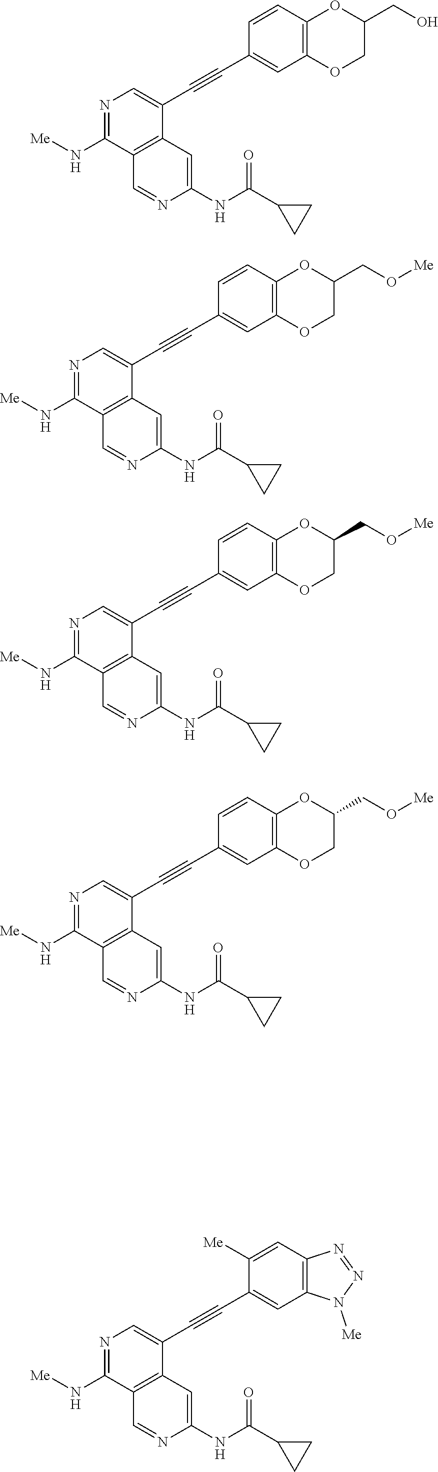

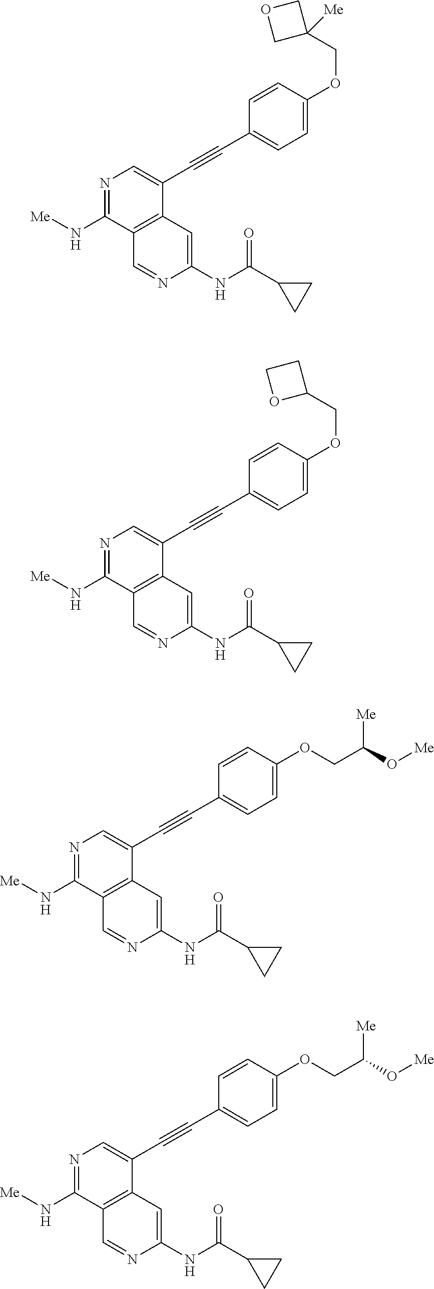 [SMILES: CNc1ncc(C#Cc2ccc(OCC3(C)COC3)cc2)c2cc(NC(=O)C3CC3)ncc12 ; CNc1ncc(C#Cc2ccc(OCC3CCO3)cc2)c2cc(NC(=O)C3CC3)ncc12 ; CNc1ncc(C#Cc2ccc(OC[C@@H](C)OC)cc2)c2cc(NC(=O)C3CC3)ncc12 ; CNc1ncc(C#Cc2ccc(OC[C@H](C)OC)cc2)c2cc(NC(=O)C3CC3)ncc12]

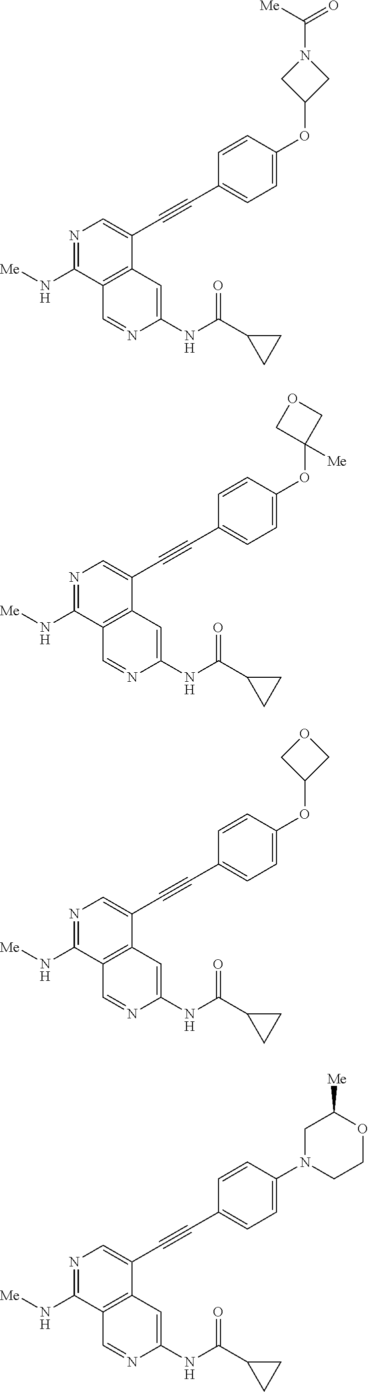

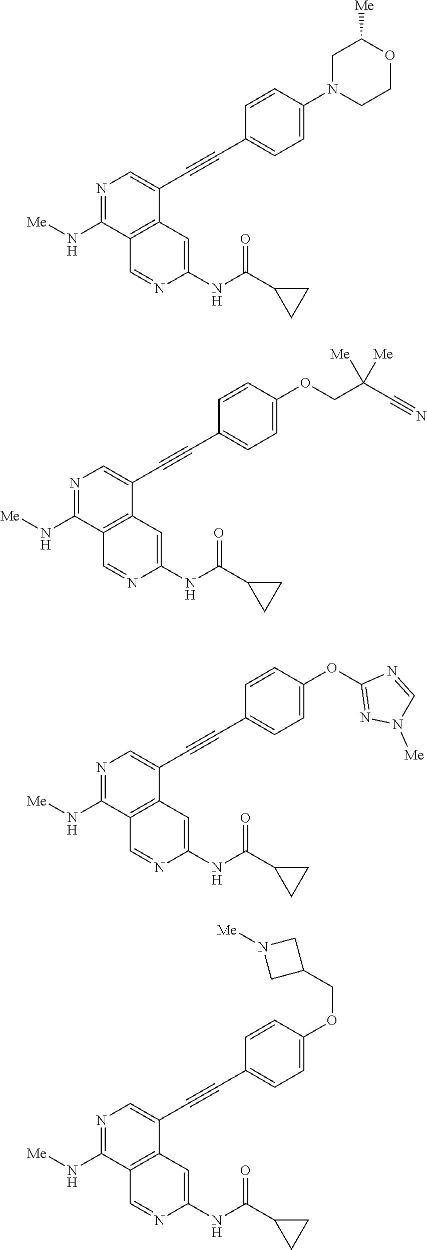

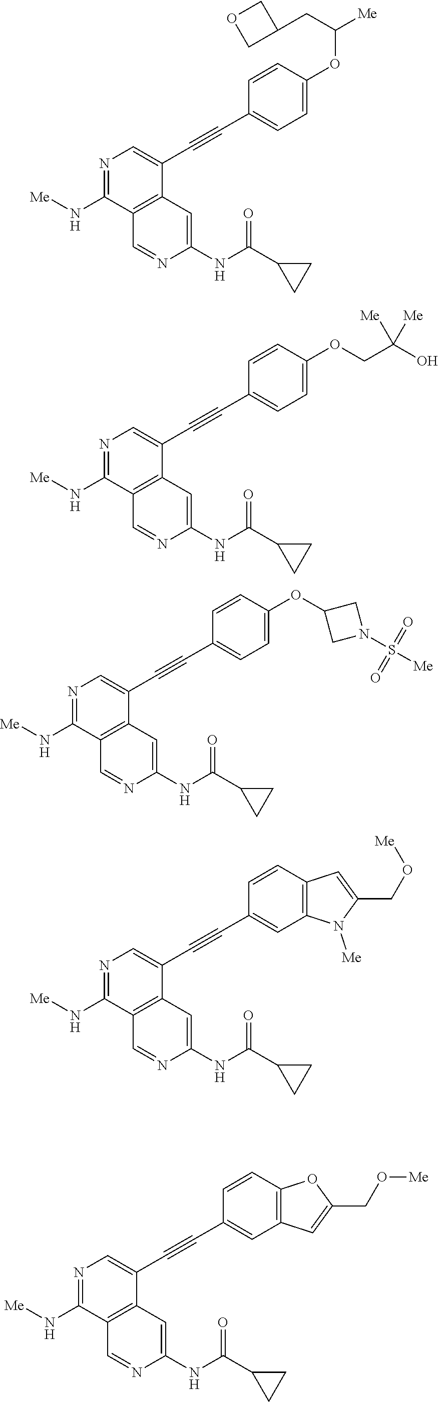

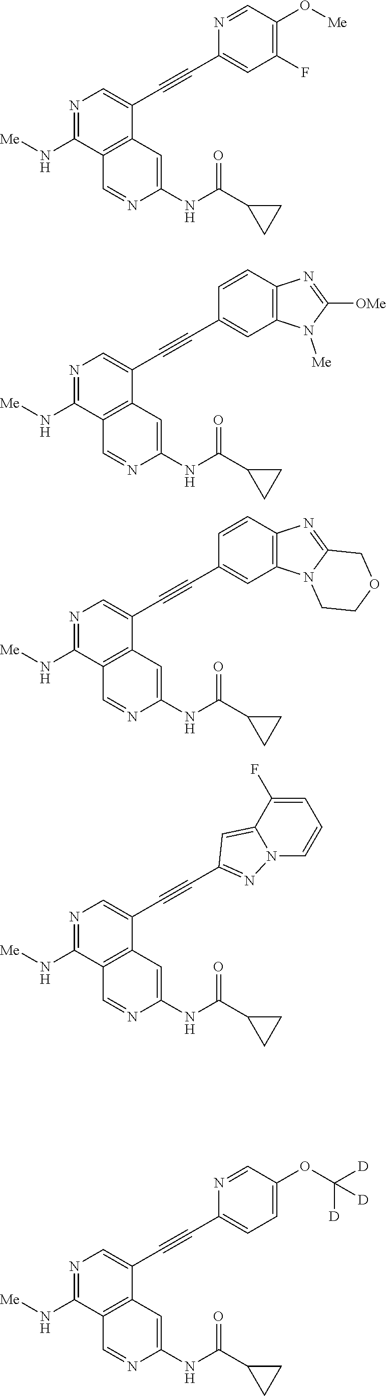

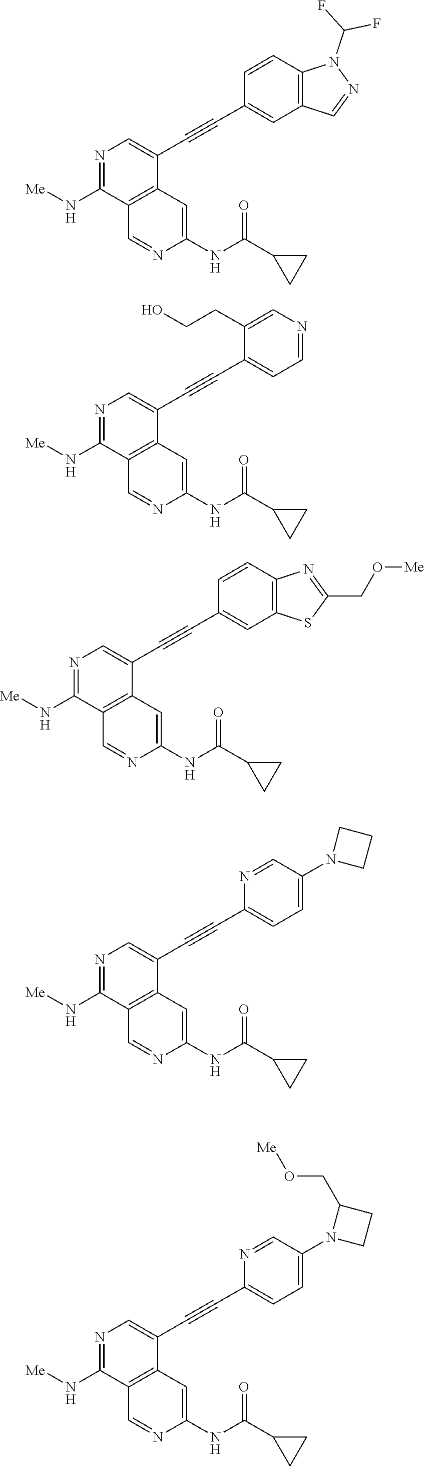

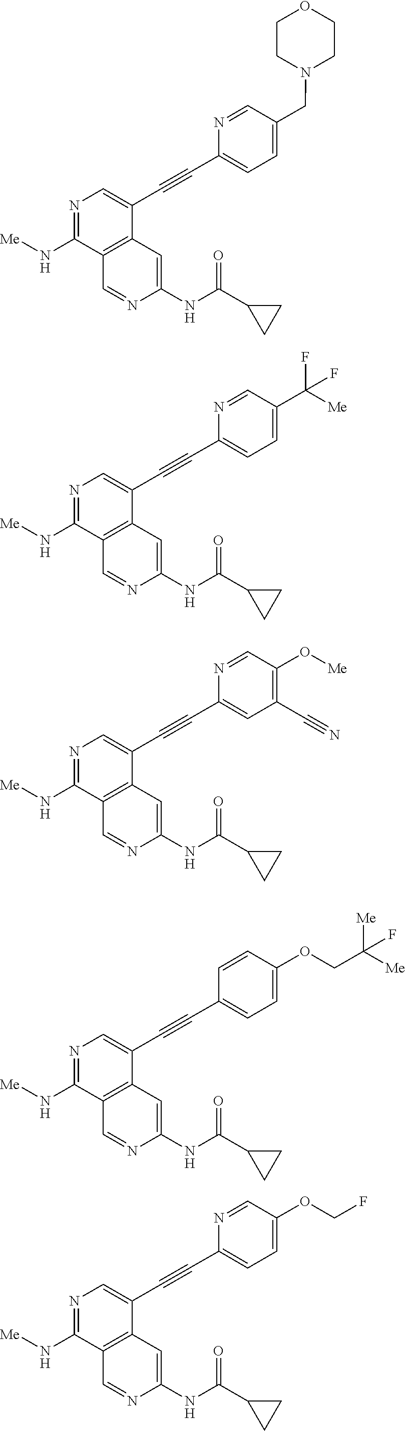

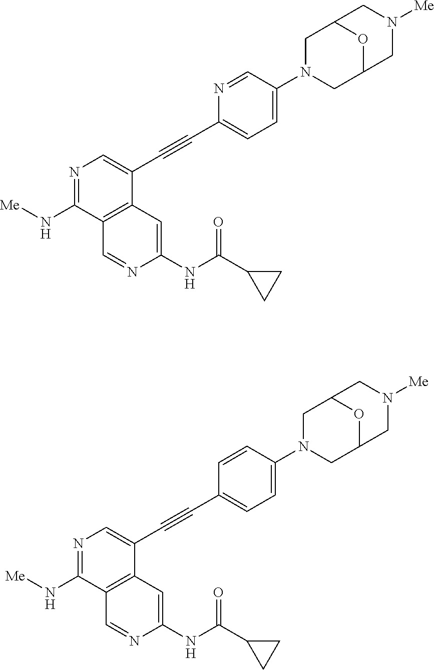 [SMILES: CNc1ncc(C#Cc2ccc(N3CC4CN(C)CC(C3)O4)cn2)c2cc(NC(=O)C3CC3)ncc12 ; CNc1ncc(C#Cc2ccc(N3CC4CN(C)CC(C3)O4)cc2)c2cc(NC(=O)C3CC3)ncc12]

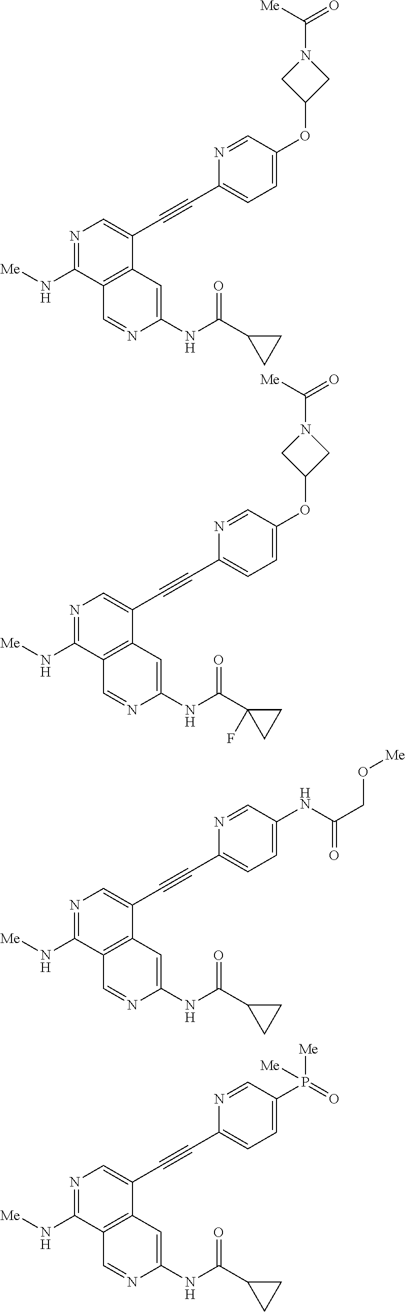

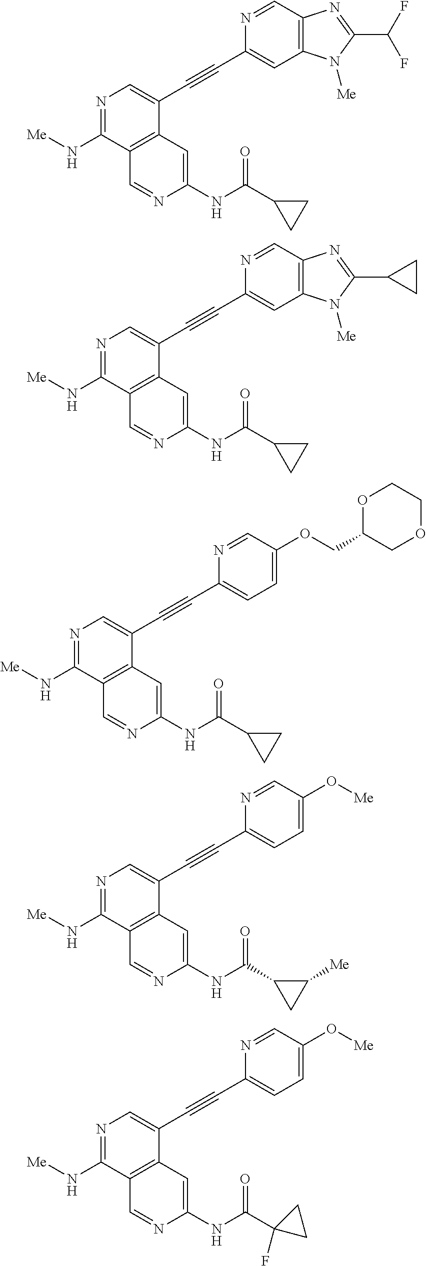

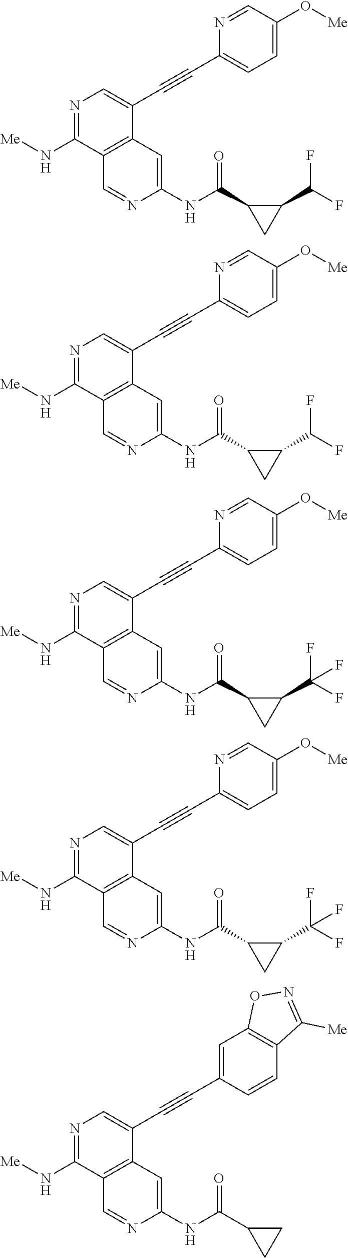

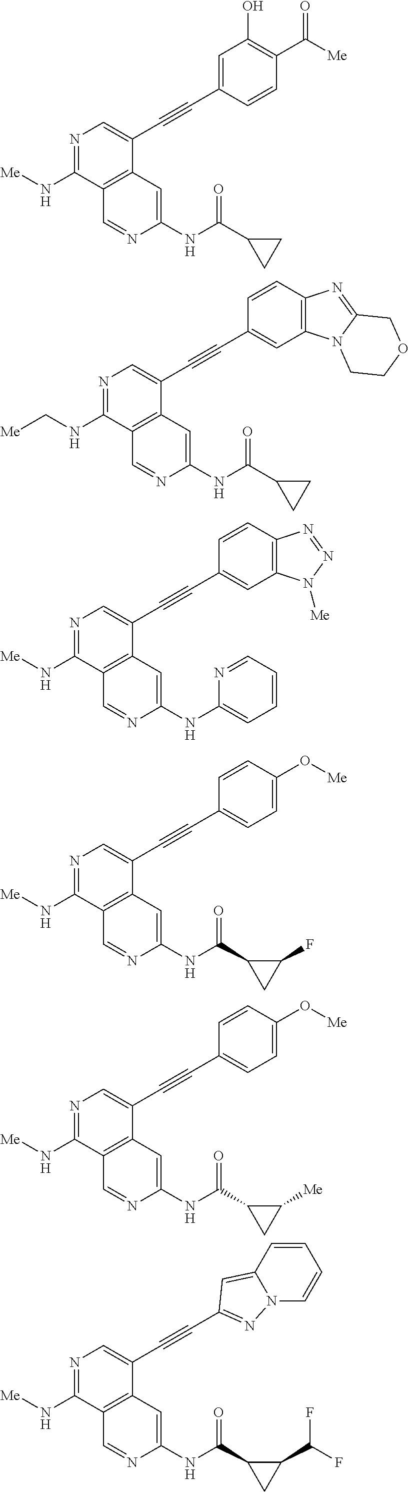

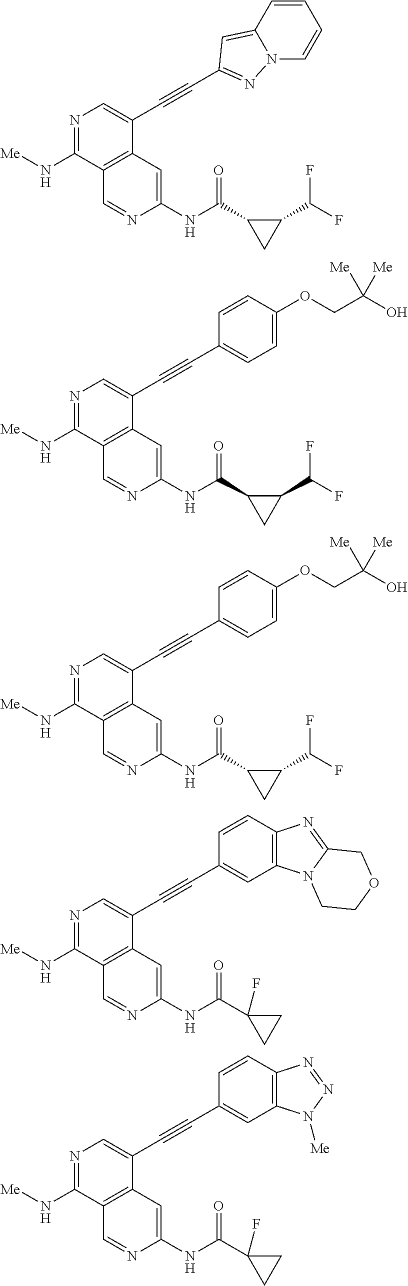

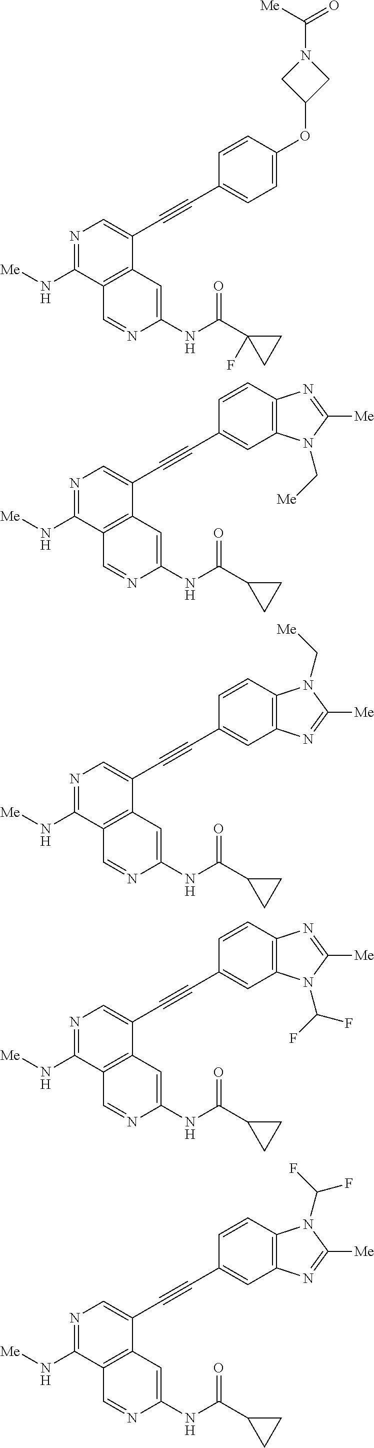

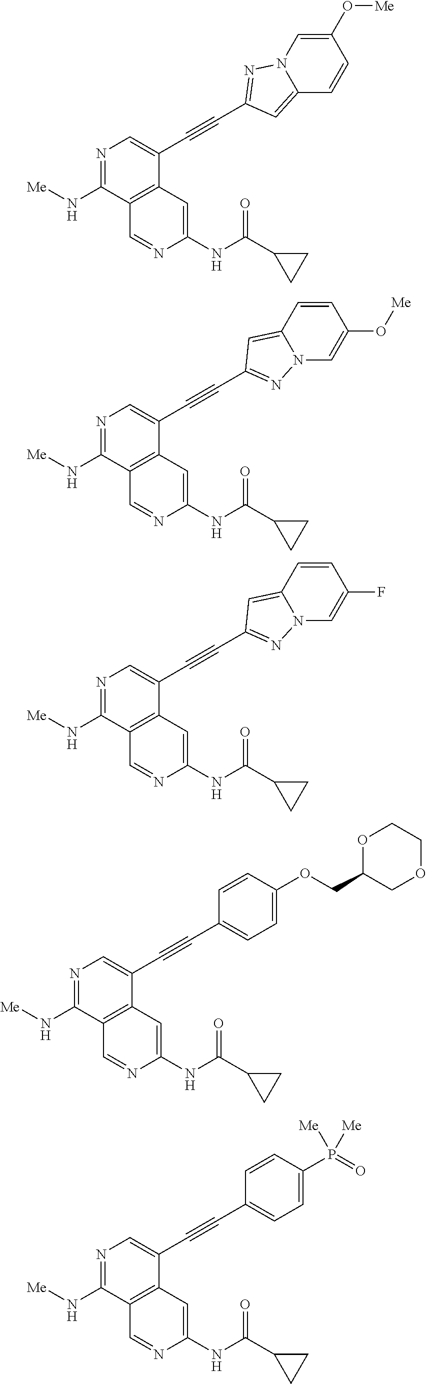

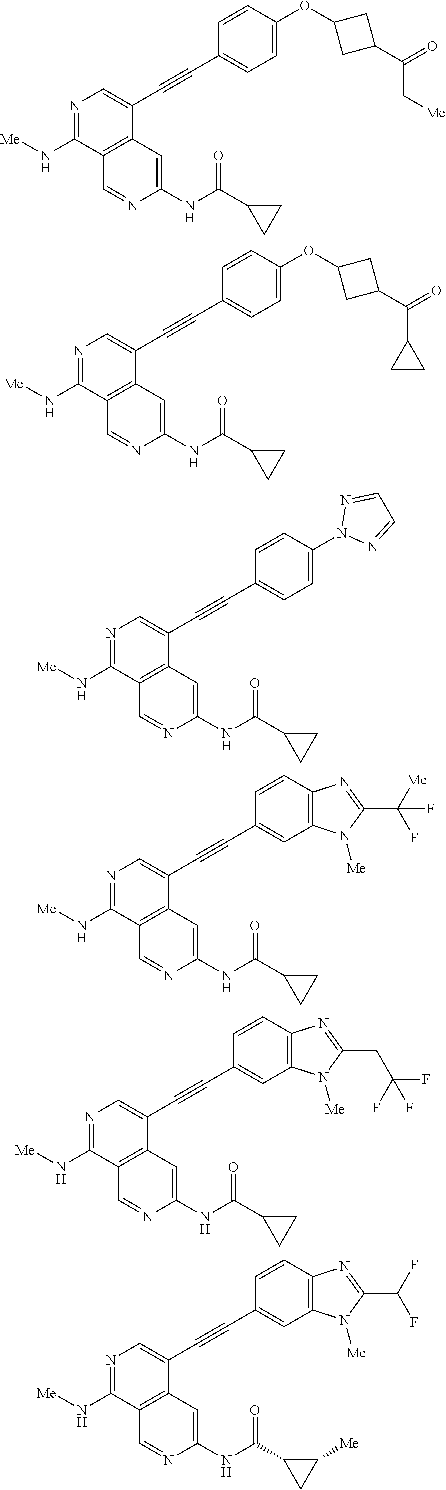

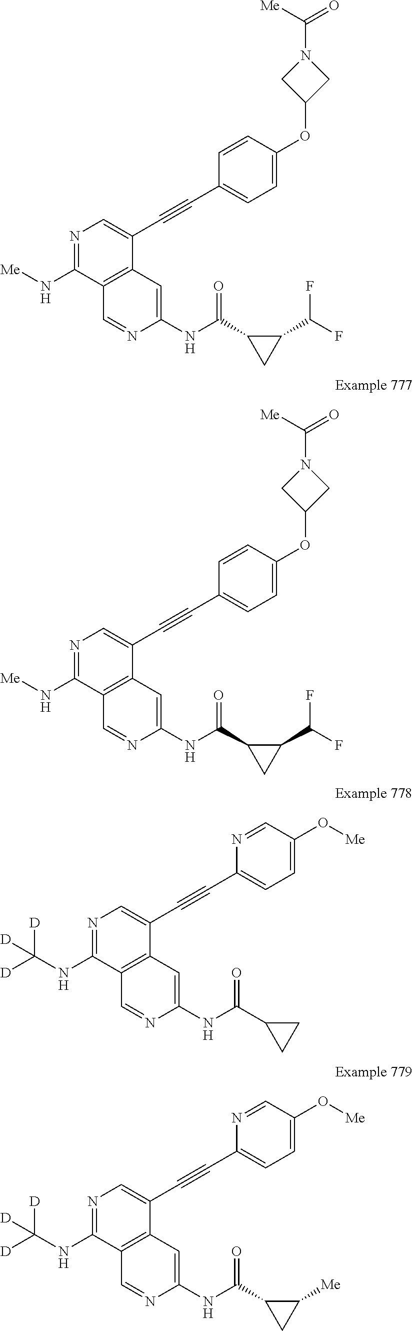 [SMILES: CNc1ncc(C#Cc2ccc(OC3CN(C(C)=O)C3)cc2)c2cc(NC(=O)[C@H]3C[C@H]3C(F)F)ncc12 ; CNc1ncc(C#Cc2ccc(OC3CN(C(C)=O)C3)cc2)c2cc(NC(=O)[C@@H]3C[C@@H]3C(F)F)ncc12 ; [2H]C([2H])([2H])Nc1ncc(C#Cc2ccc(OC)cn2)c2cc(NC(=O)C3CC3)ncc12 ; [2H]C([2H])([2H])Nc1ncc(C#Cc2ccc(OC)cn2)c2cc(NC(=O)[C@H]3C[C@H]3C)ncc12]

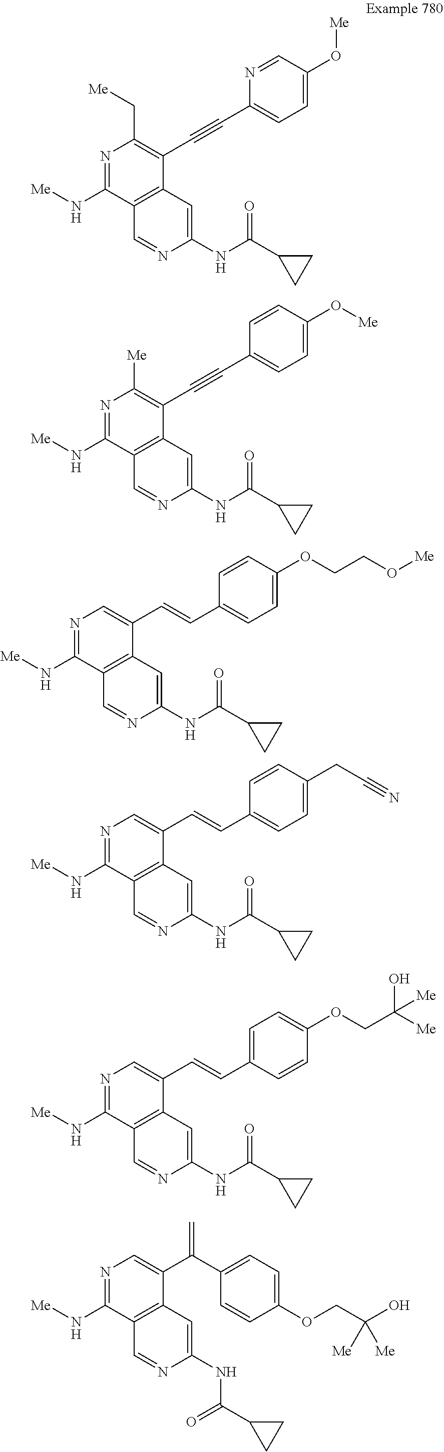

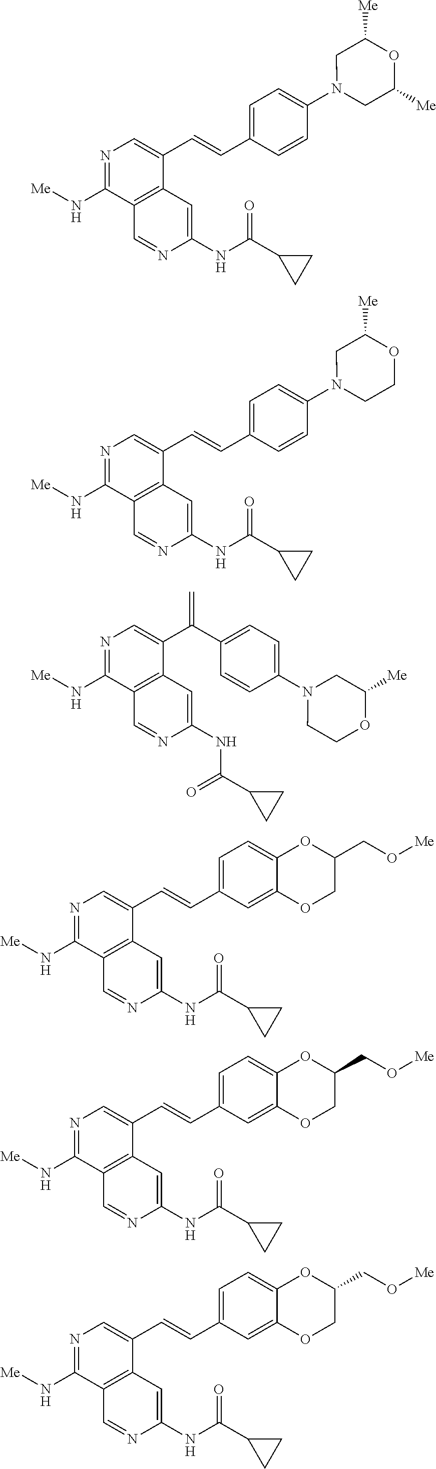

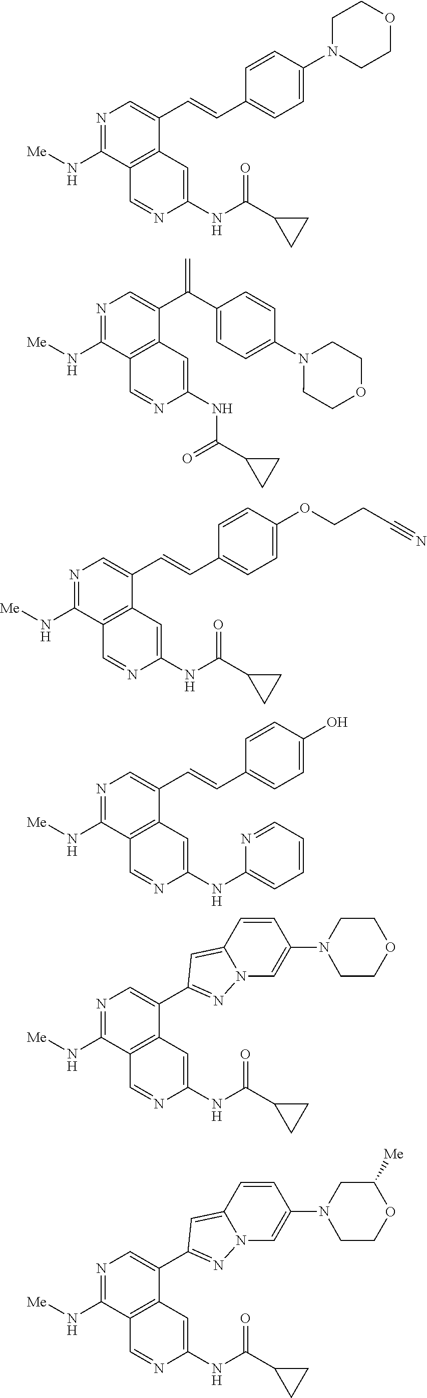

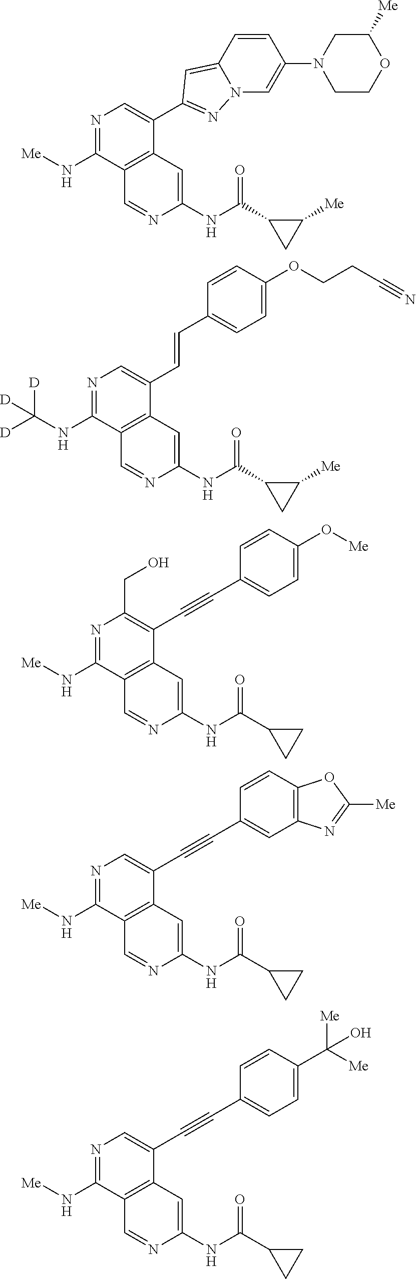

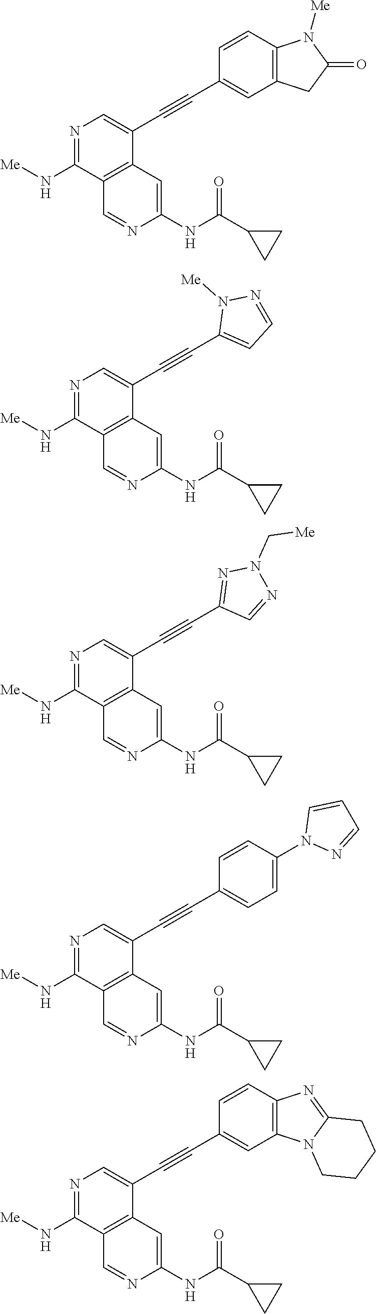

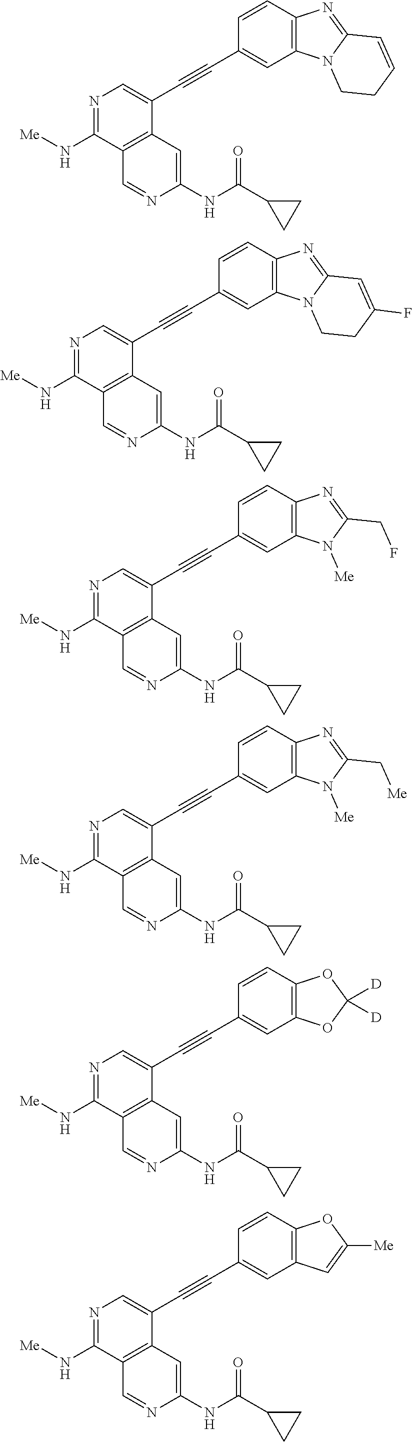

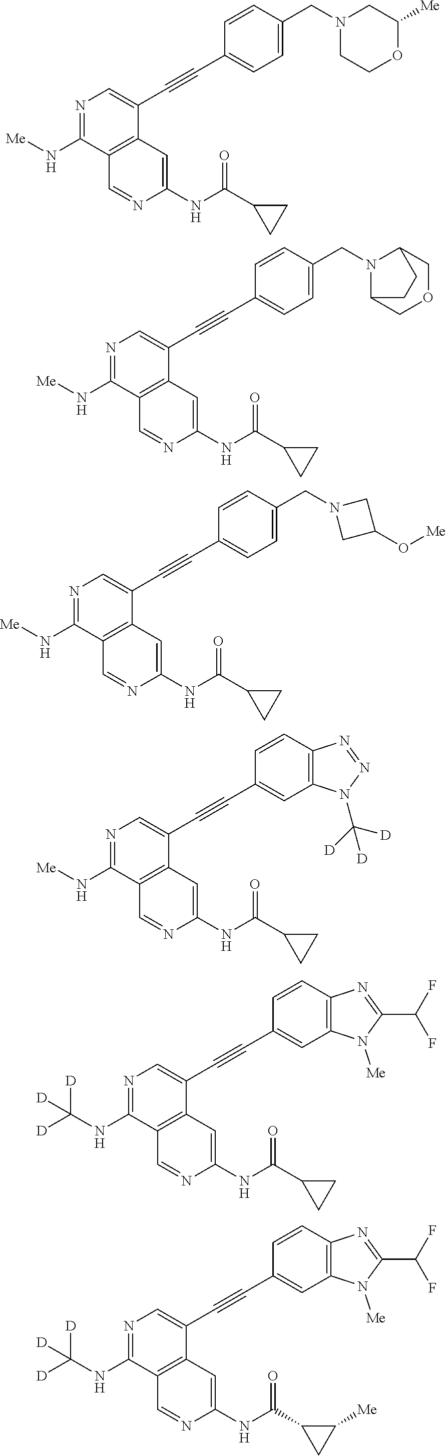

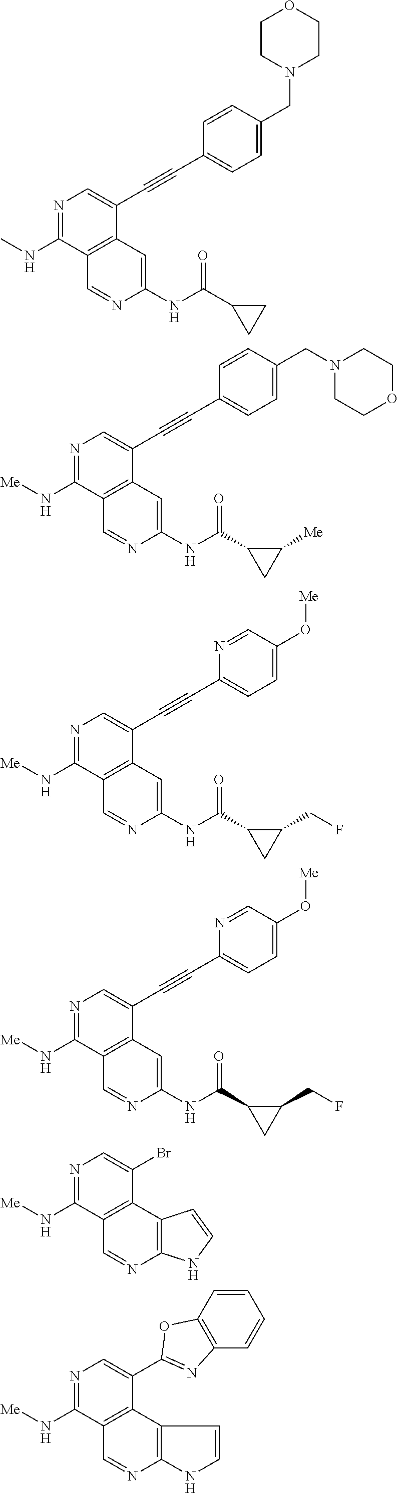

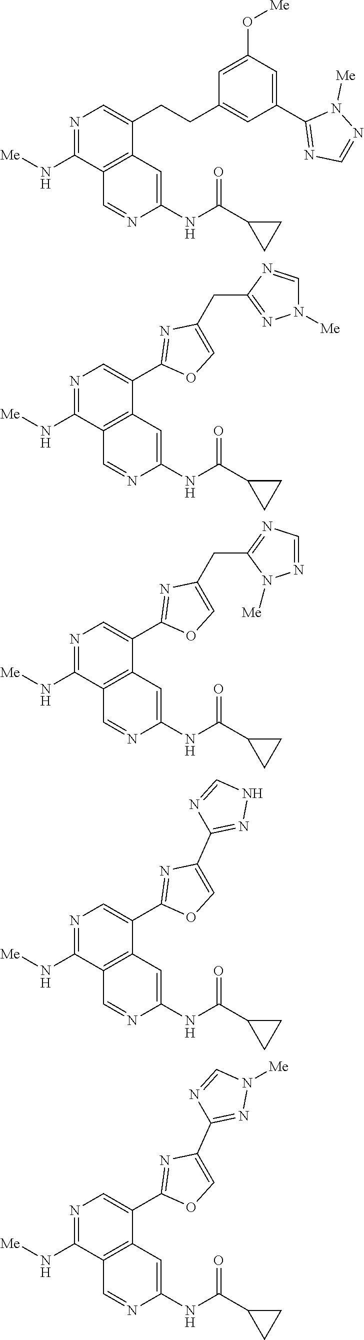

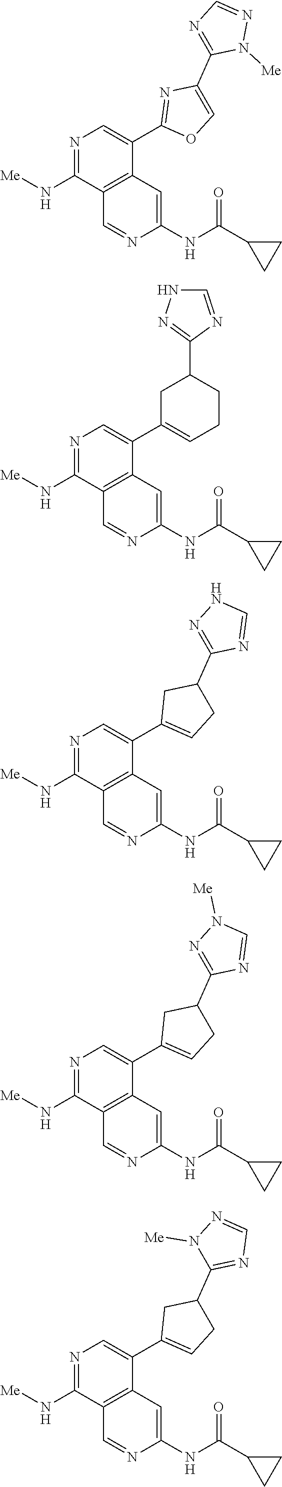

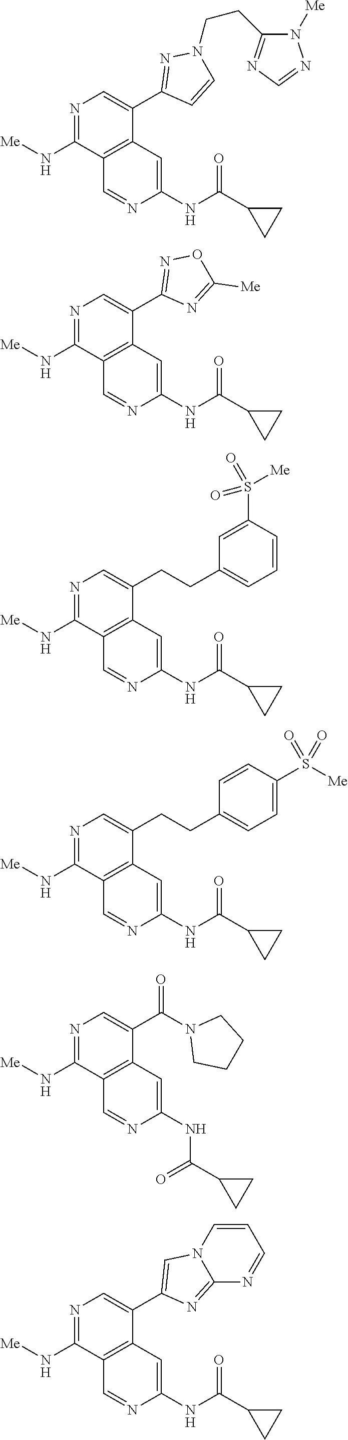

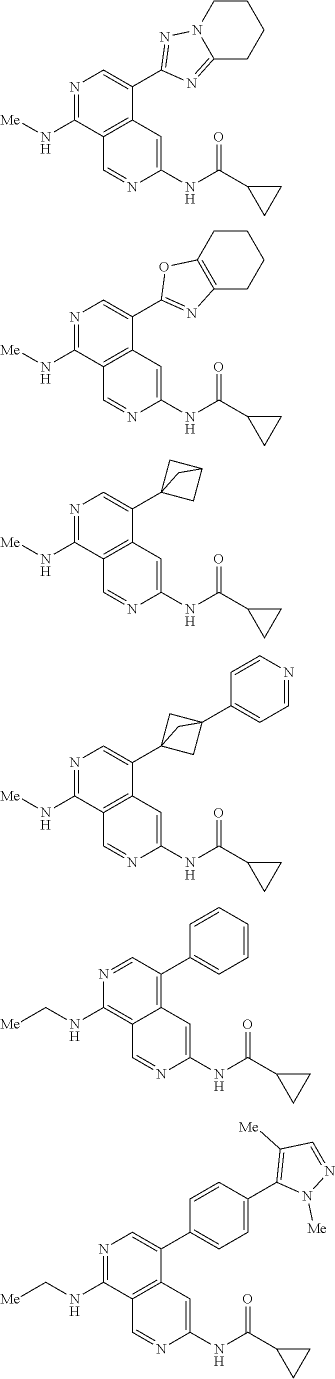

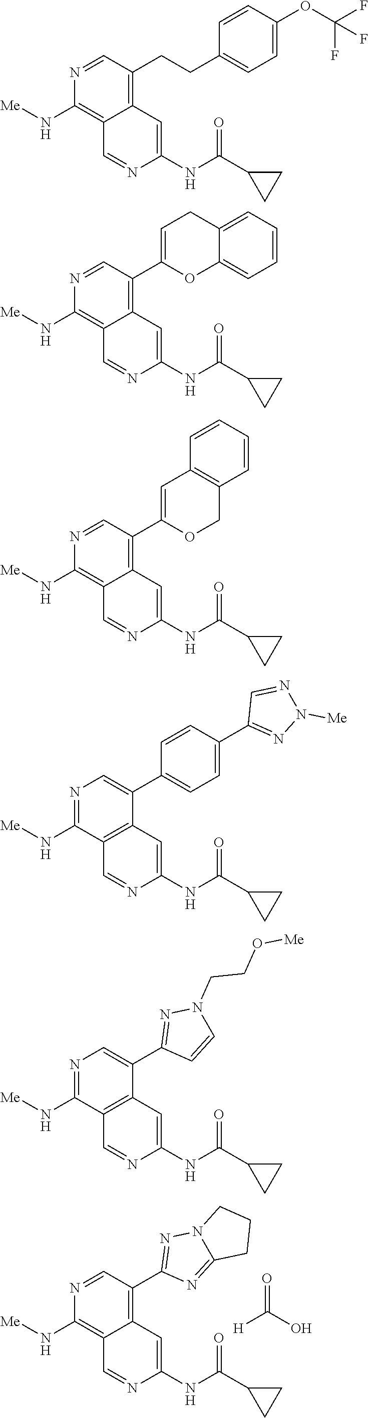

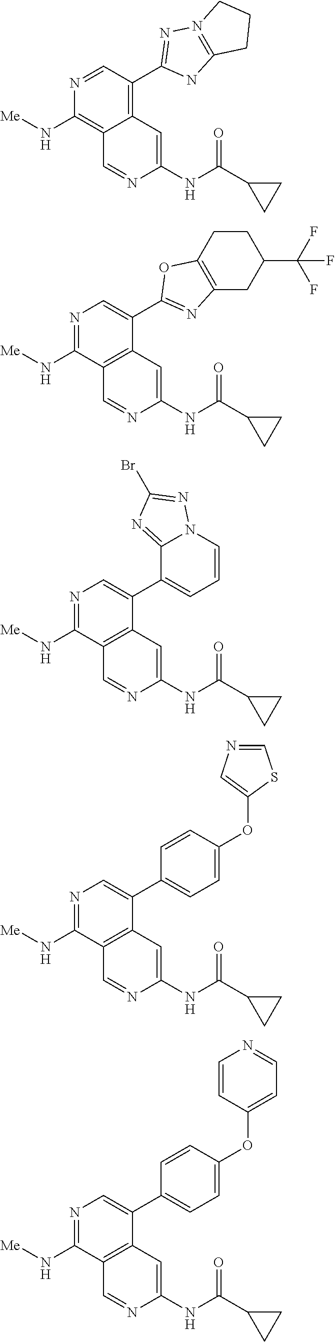

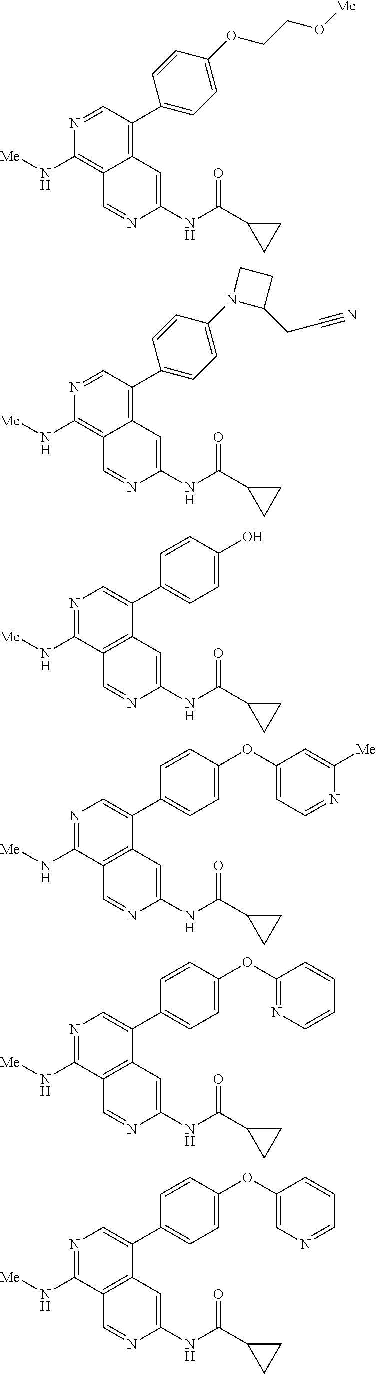

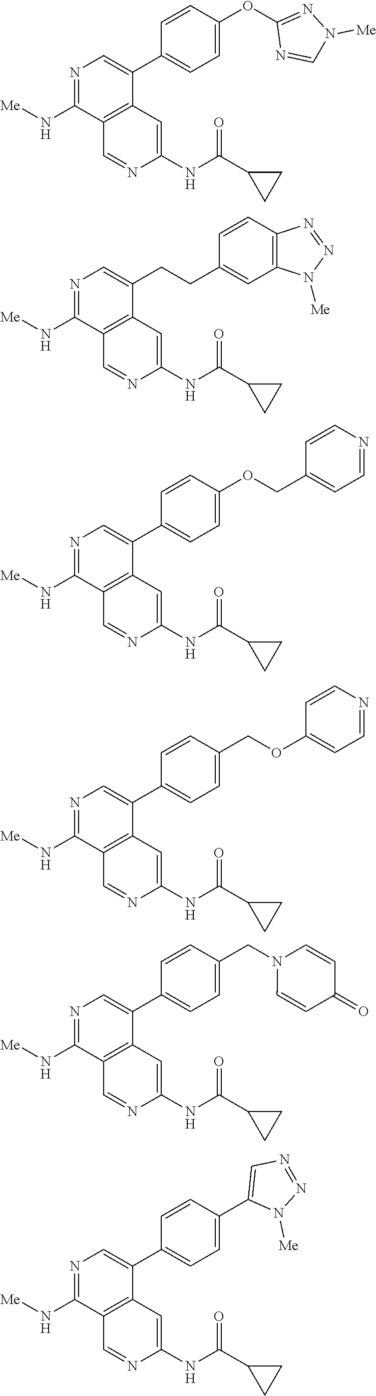

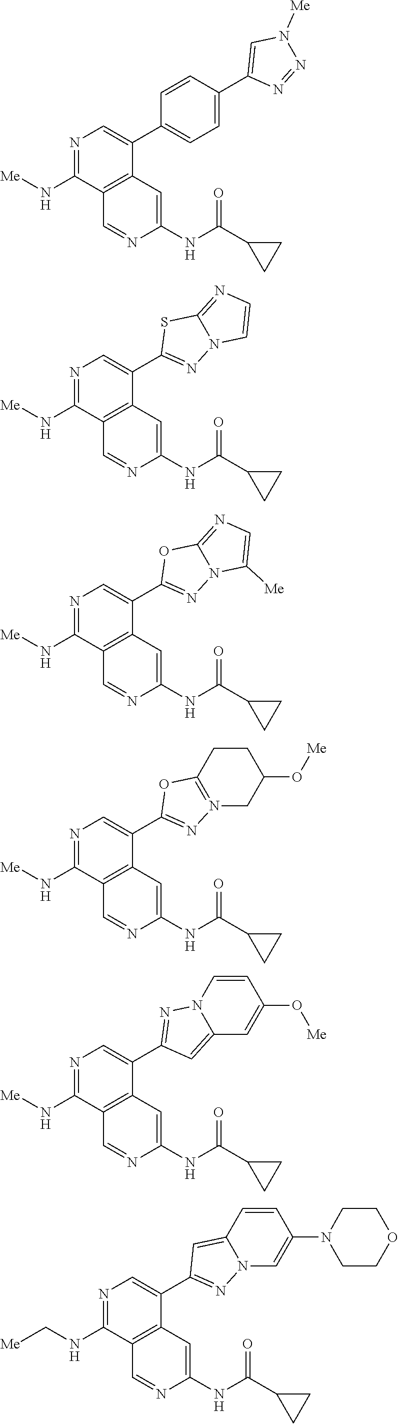

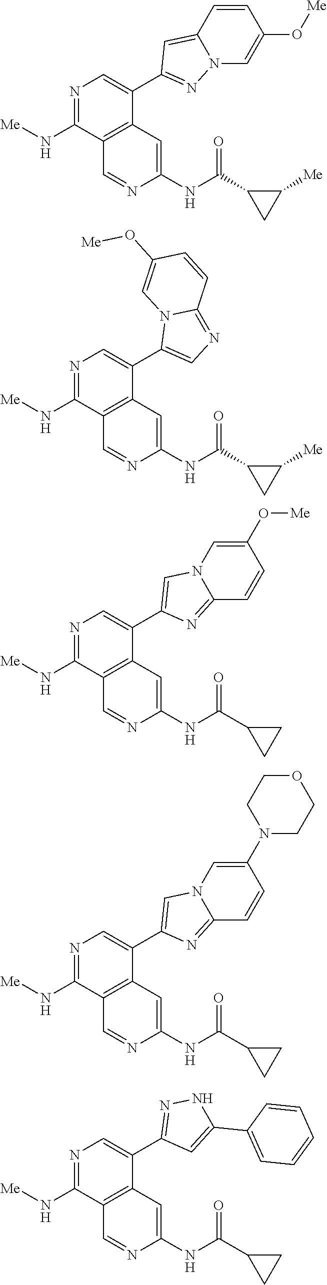

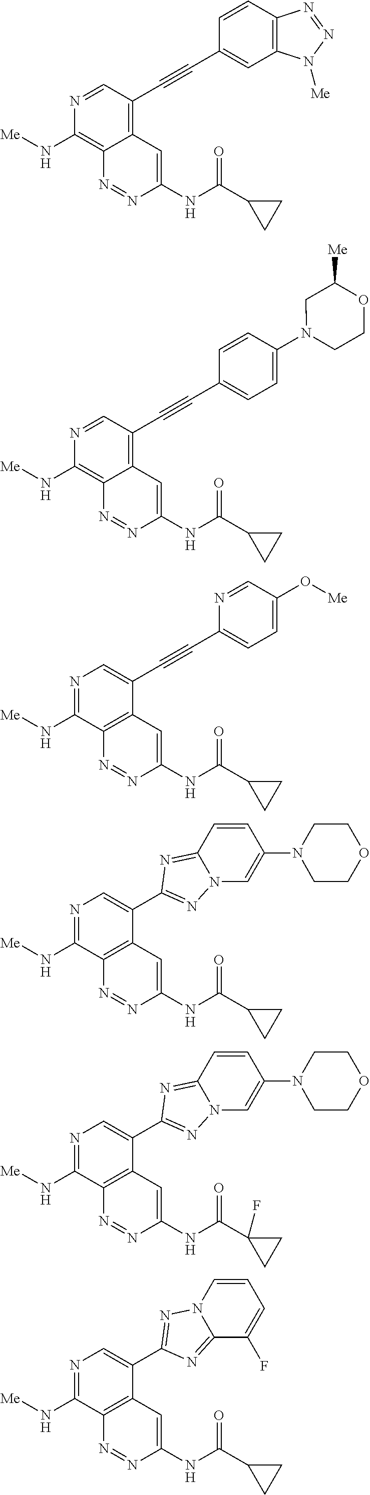

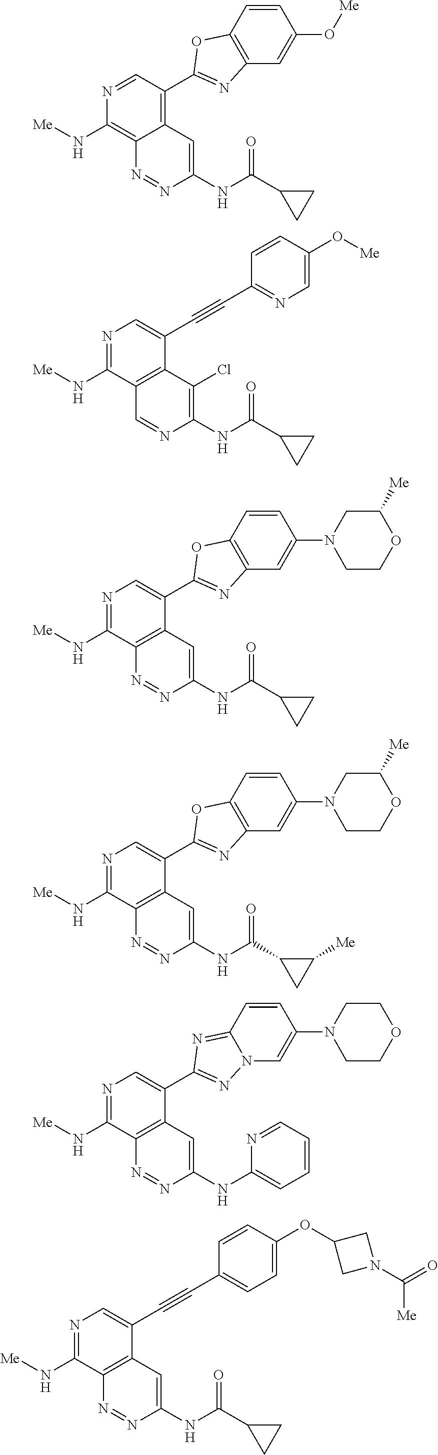

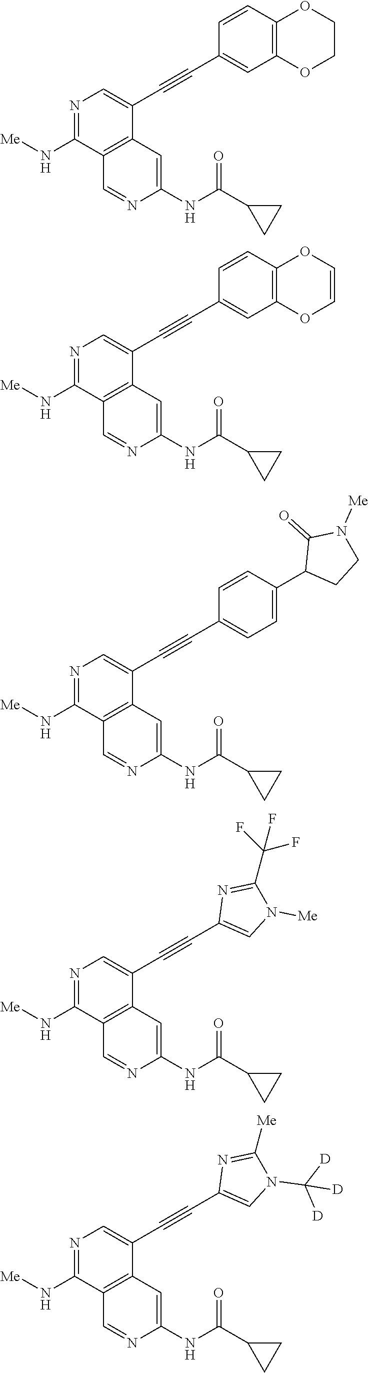

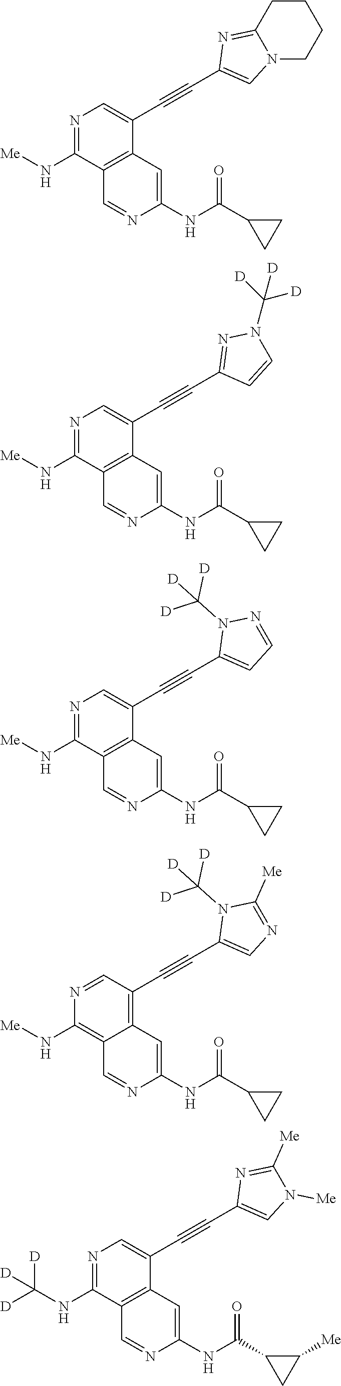

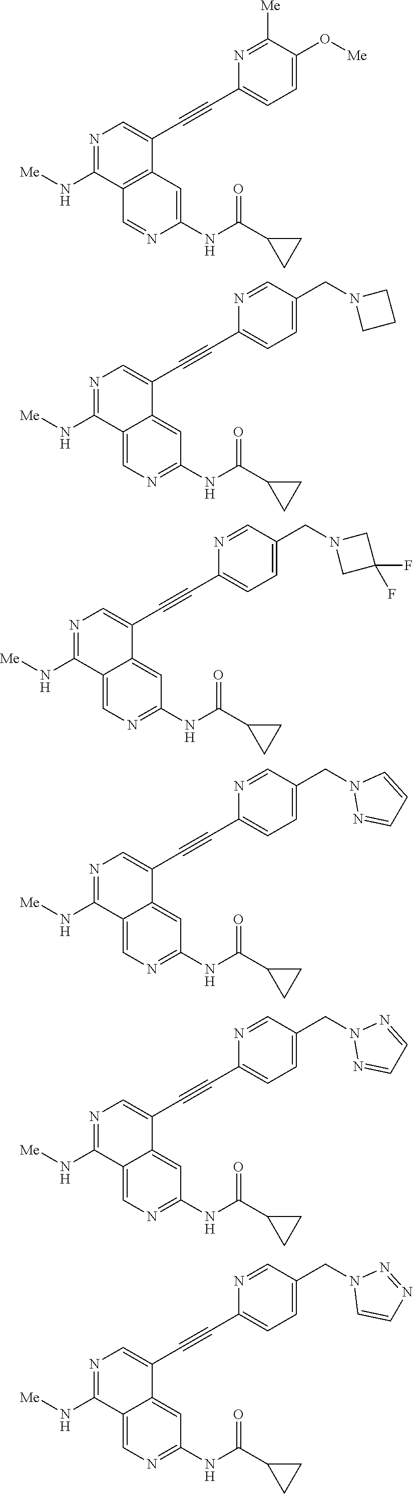

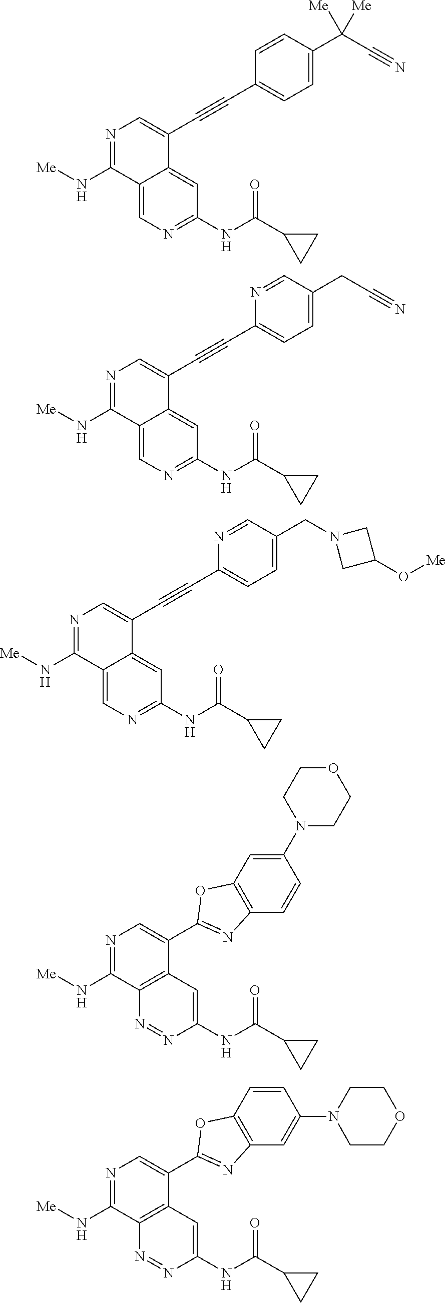

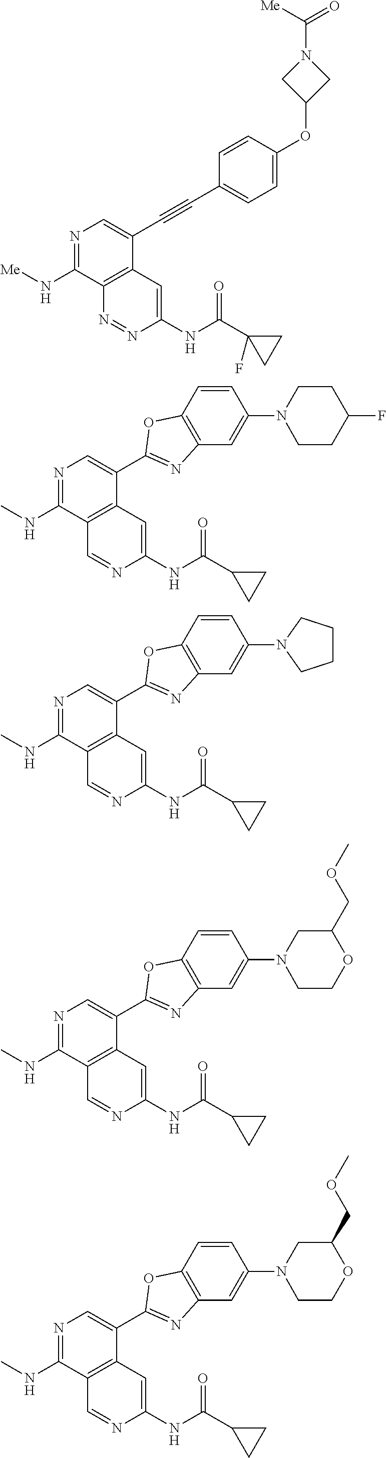

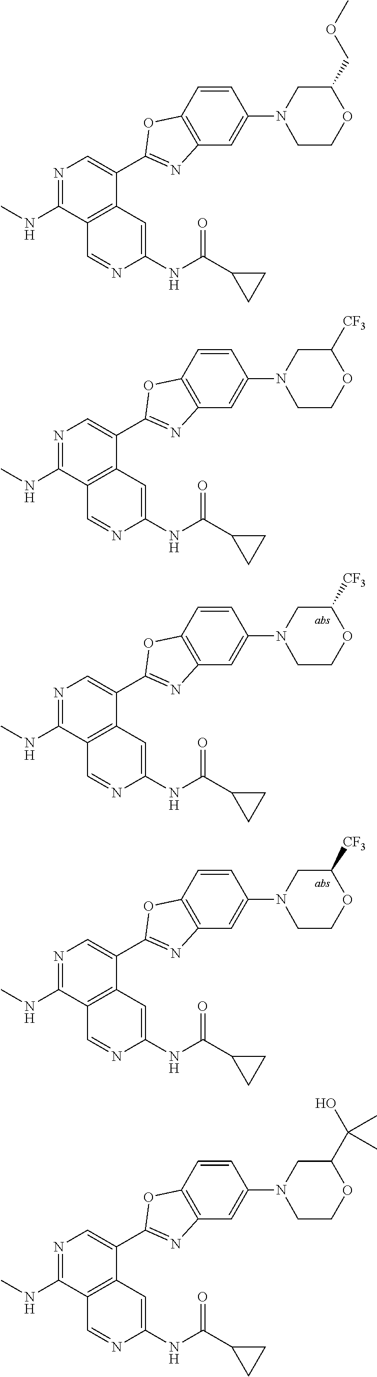

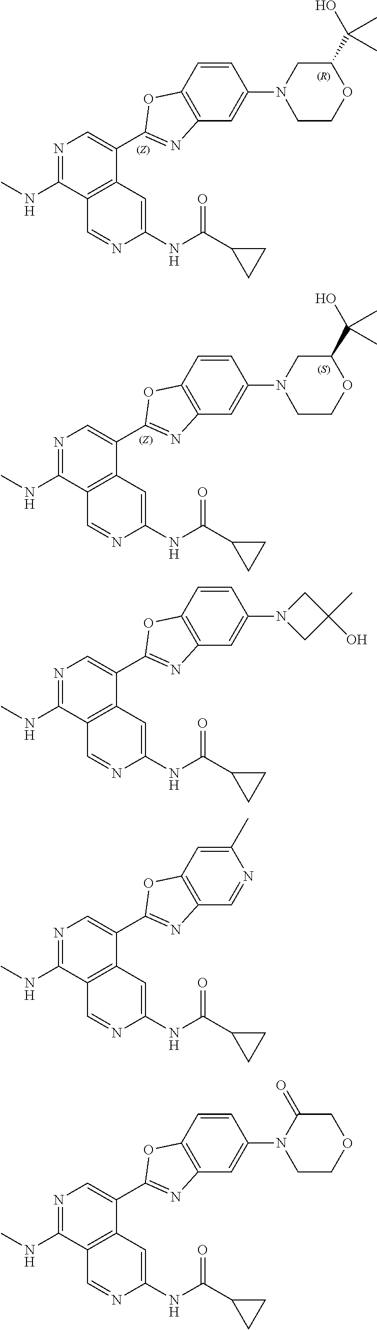

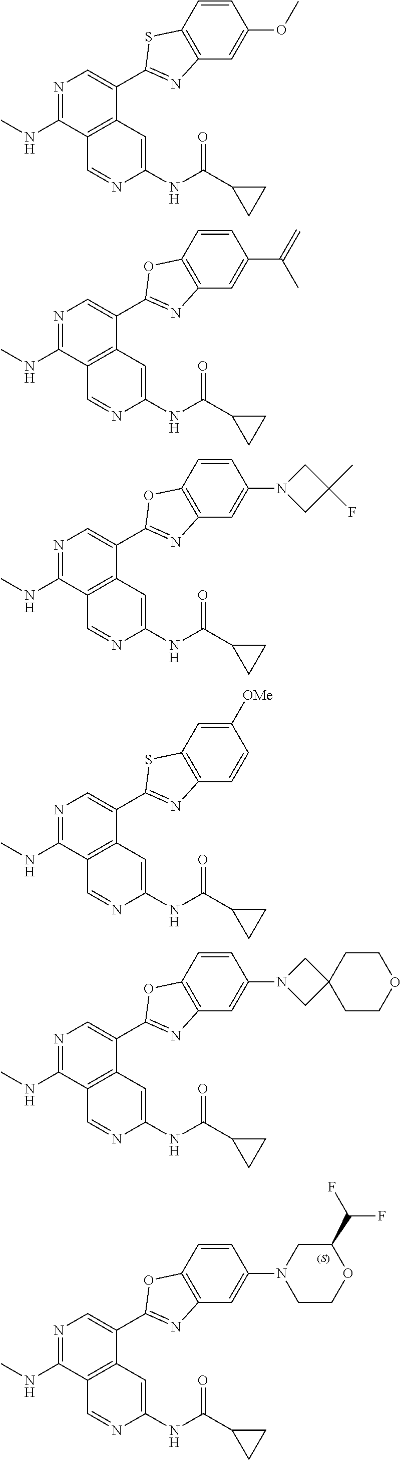

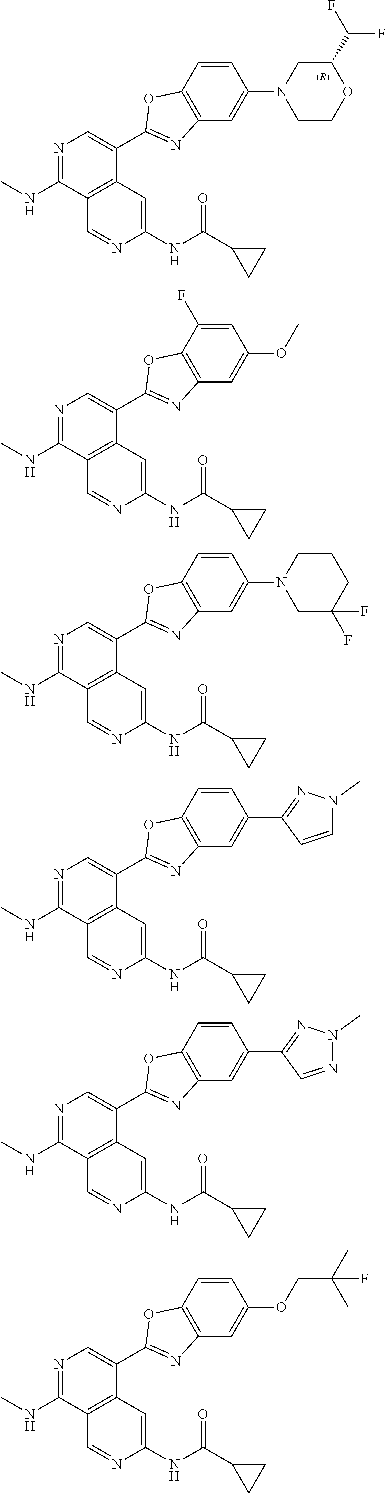

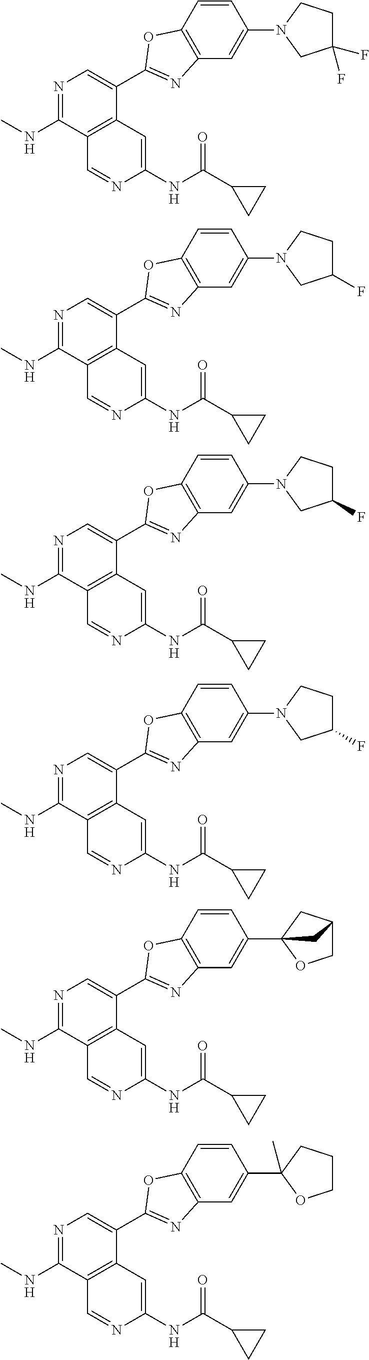

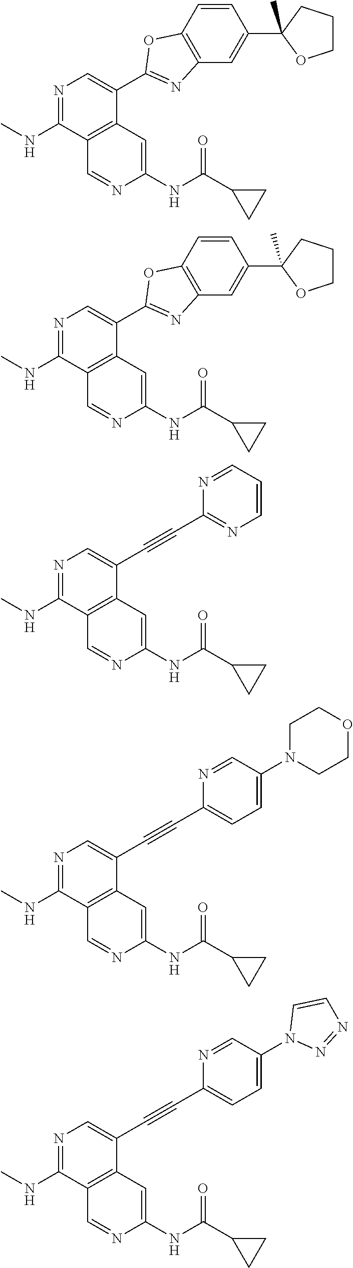

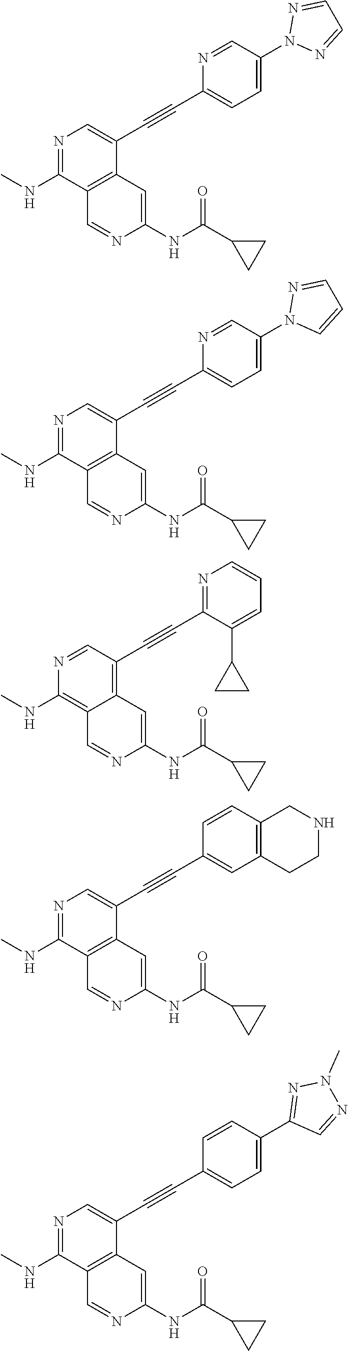

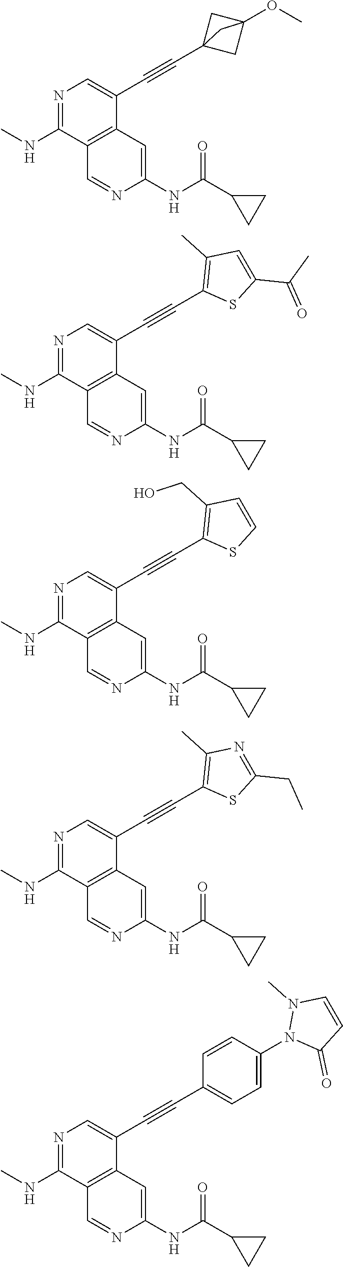

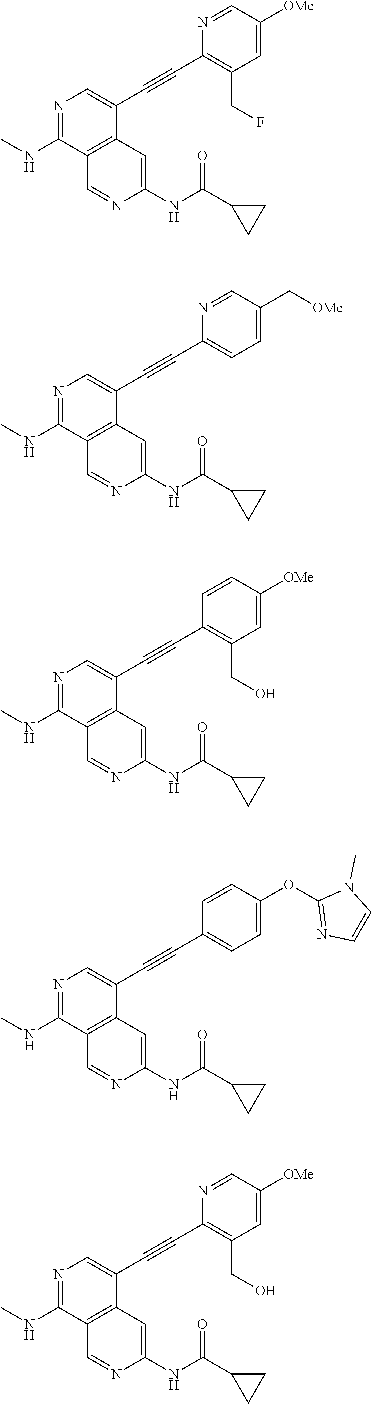 [SMILES: CNc1ncc(C#Cc2ncc(OC)cc2CF)c2cc(NC(=O)C3CC3)ncc12 ; CNc1ncc(C#Cc2ccc(COC)cn2)c2cc(NC(=O)C3CC3)ncc12 ; CNc1ncc(C#Cc2ccc(OC)cc2CO)c2cc(NC(=O)C3CC3)ncc12 ; CNc1ncc(C#Cc2ccc(Oc3nccn3C)cc2)c2cc(NC(=O)C3CC3)ncc12 ; CNc1ncc(C#Cc2ncc(OC)cc2CO)c2cc(NC(=O)C3CC3)ncc12]

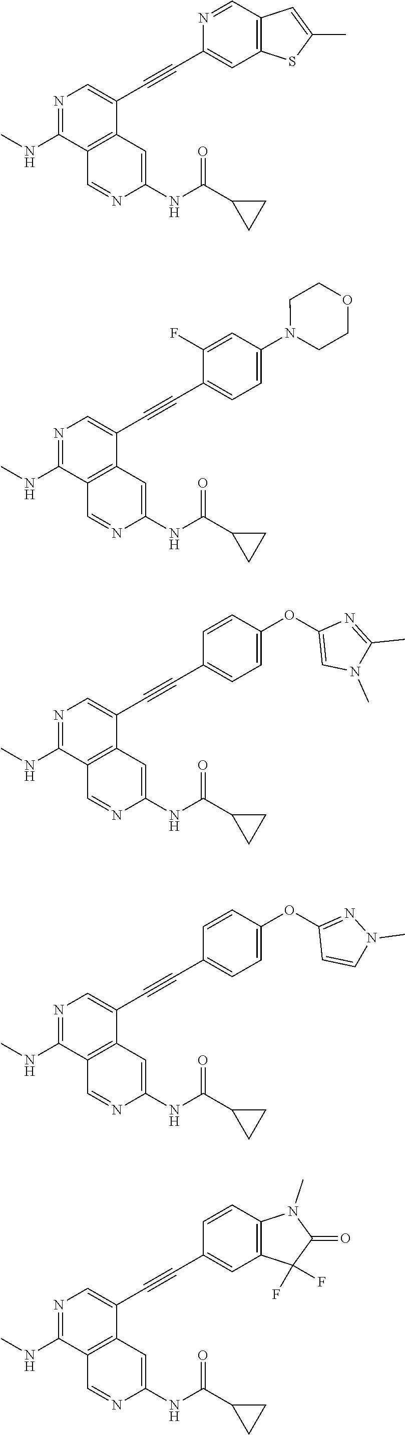

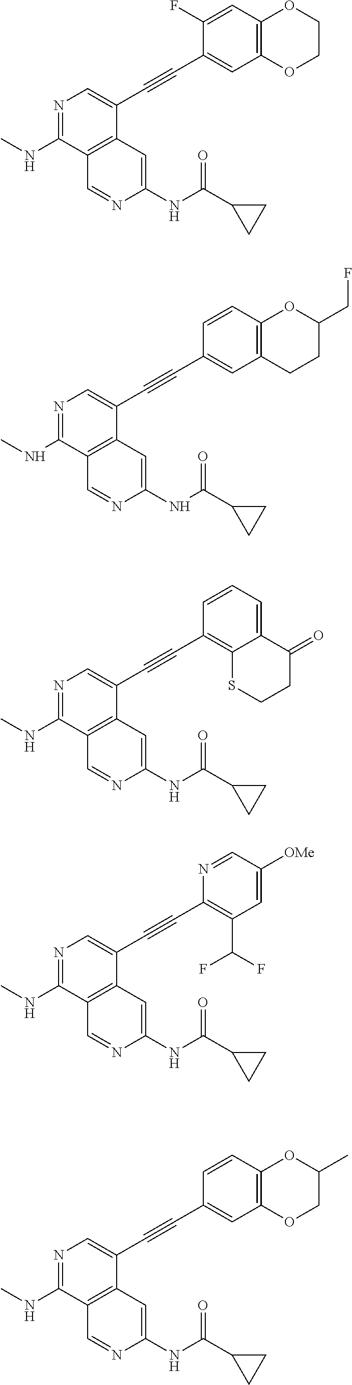

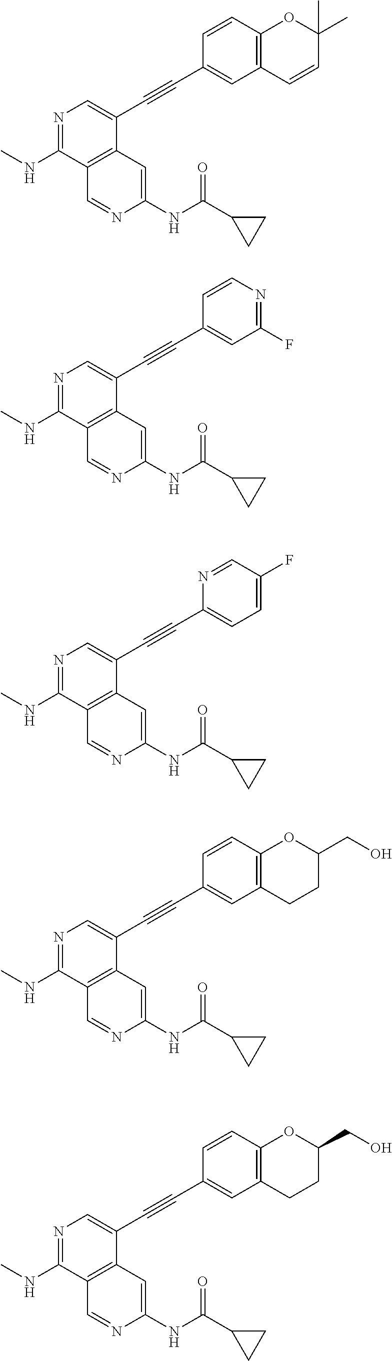

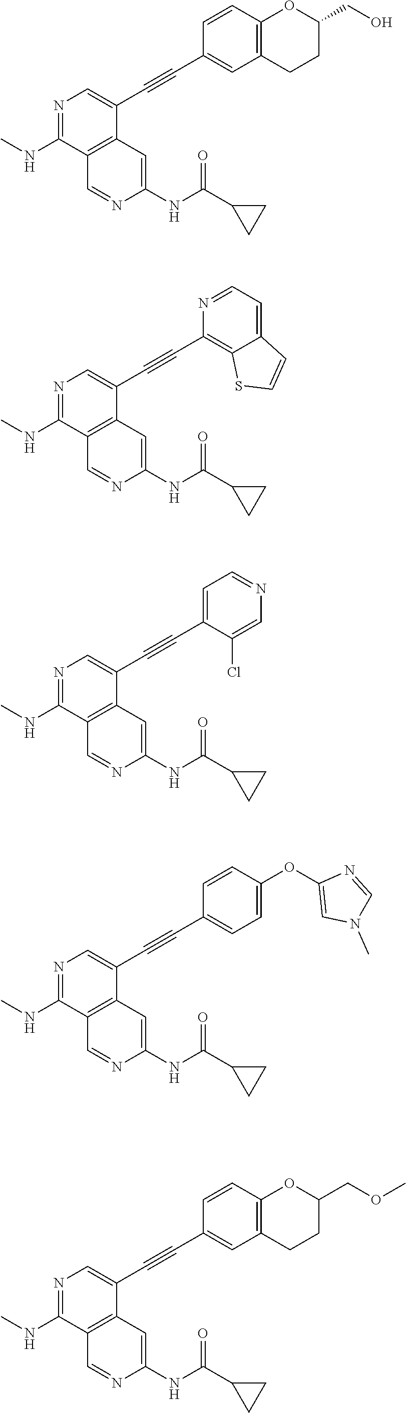

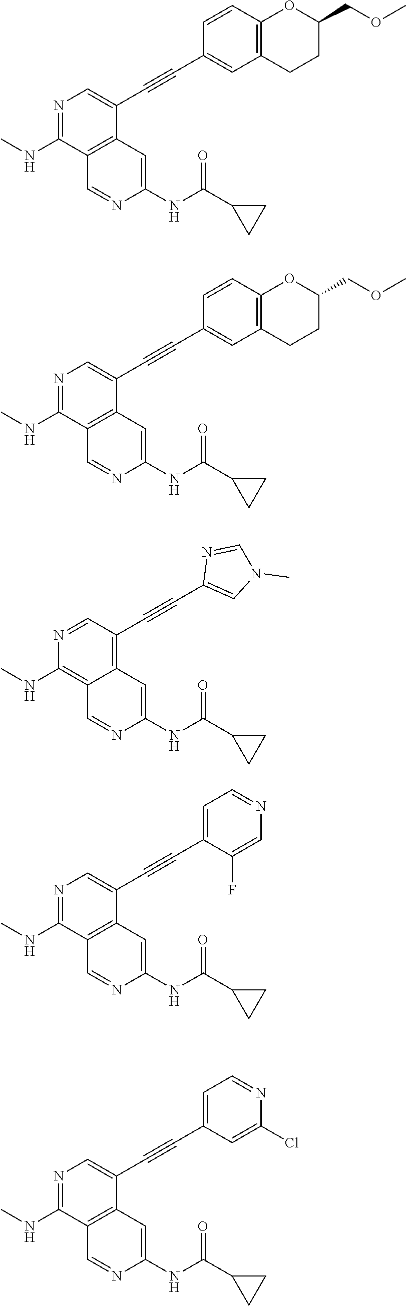

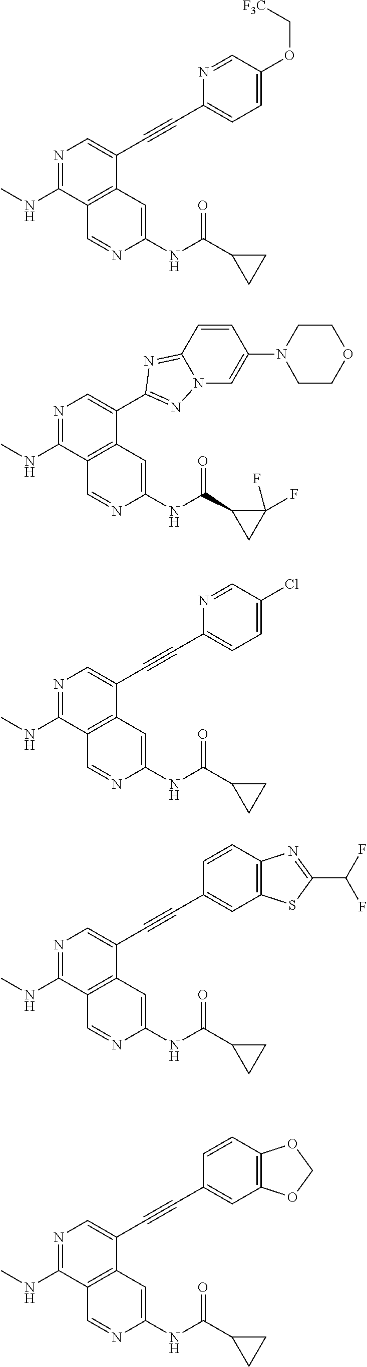

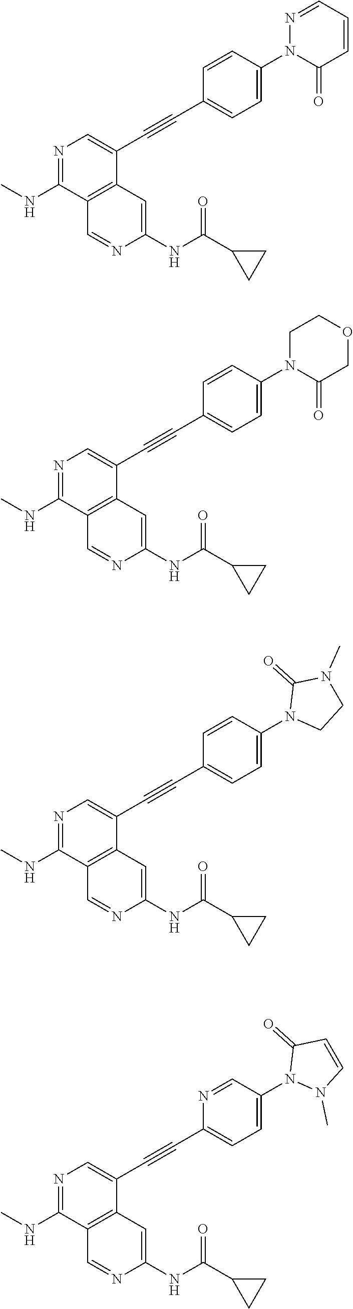 [SMILES: CNc1ncc(C#Cc2ccc(-n3ncccc3=O)cc2)c2cc(NC(=O)C3CC3)ncc12 ; CNc1ncc(C#Cc2ccc(N3CCOCC3=O)cc2)c2cc(NC(=O)C3CC3)ncc12 ; CNc1ncc(C#Cc2ccc(N3CCN(C)C3=O)cc2)c2cc(NC(=O)C3CC3)ncc12 ; CNc1ncc(C#Cc2ccc(-n3c(=O)ccn3C)cn2)c2cc(NC(=O)C3CC3)ncc12]

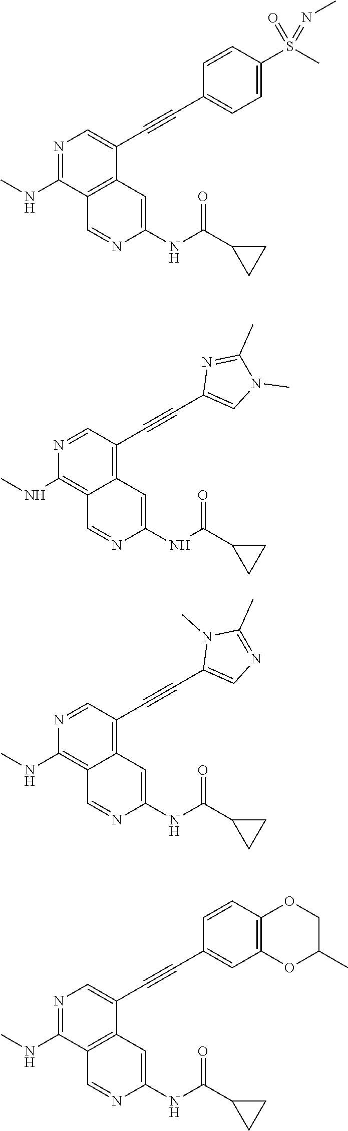

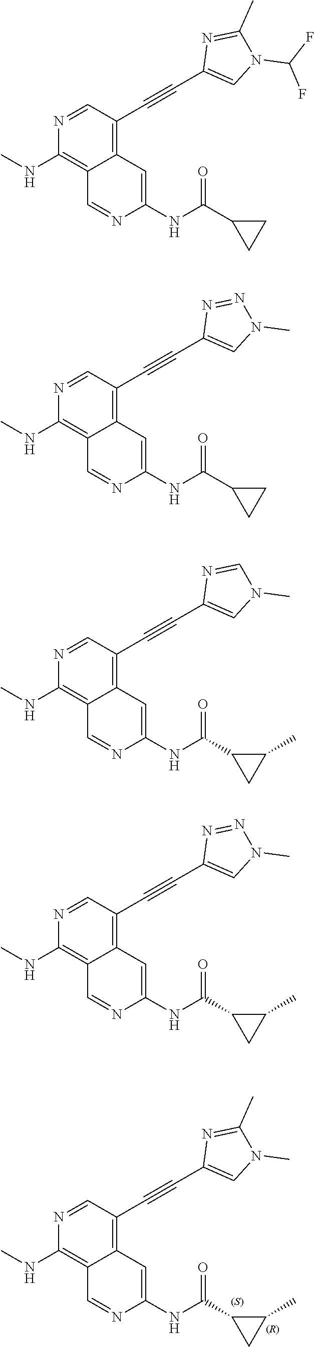

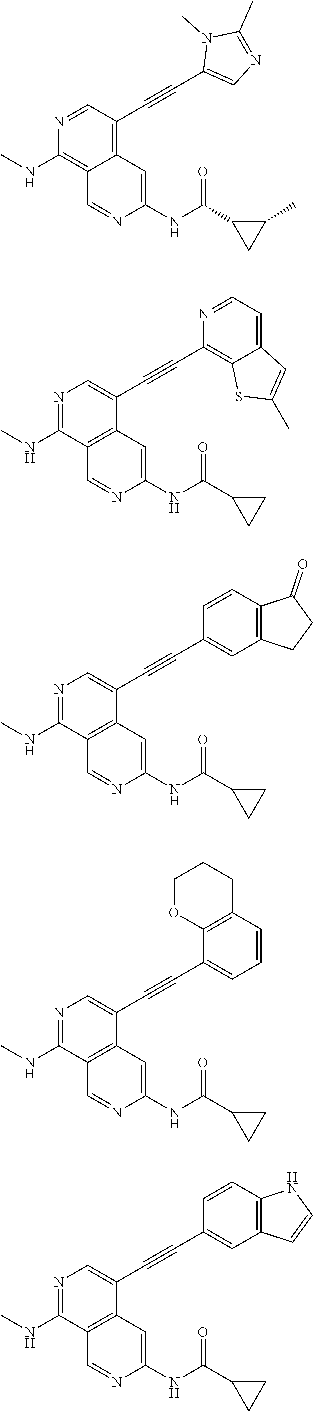

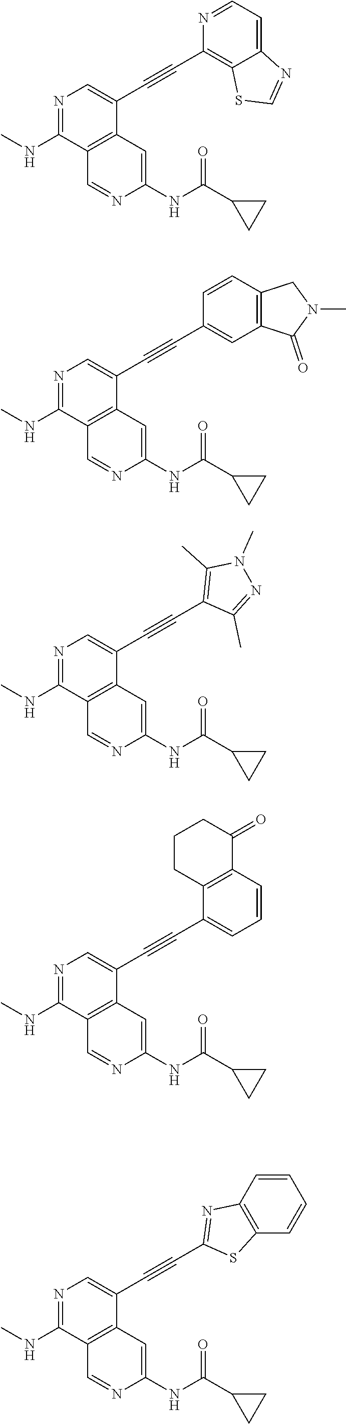

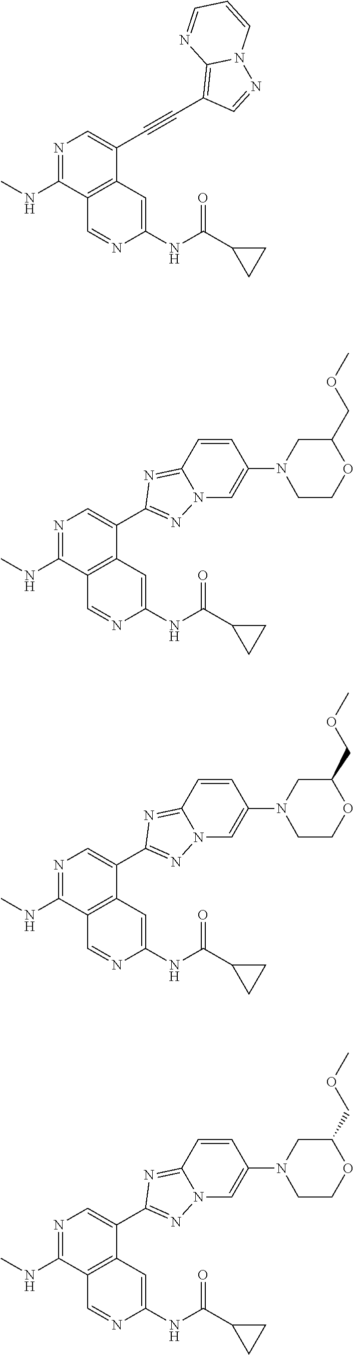

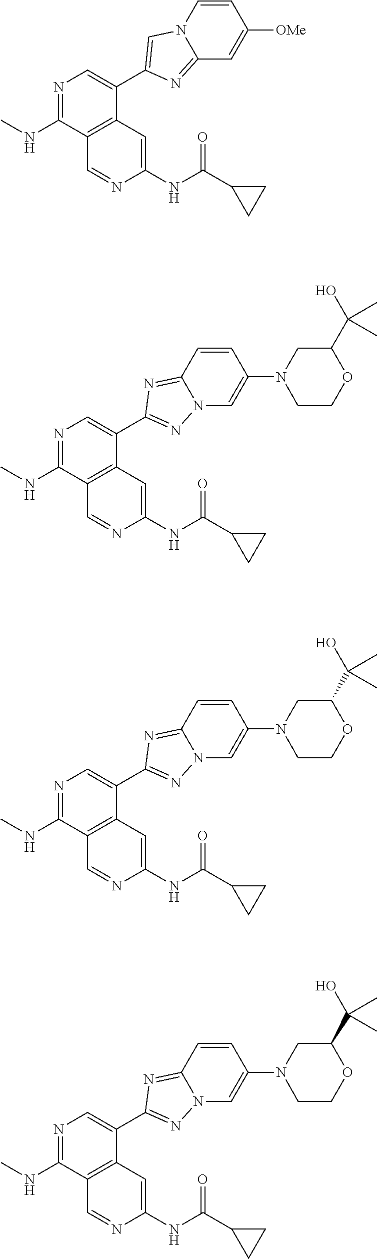

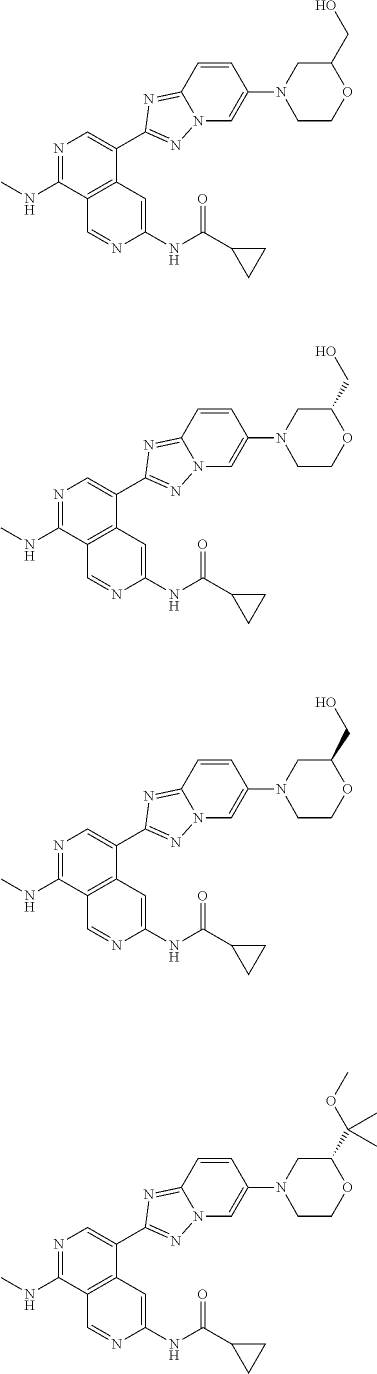 [SMILES: CNc1ncc(-c2nc3ccc(N4CCOC(CO)C4)cn3n2)c2cc(NC(=O)C3CC3)ncc12 ; CNc1ncc(-c2nc3ccc(N4CCO[C@@H](CO)C4)cn3n2)c2cc(NC(=O)C3CC3)ncc12 ; CNc1ncc(-c2nc3ccc(N4CCO[C@H](CO)C4)cn3n2)c2cc(NC(=O)C3CC3)ncc12 ; CNc1ncc(-c2nc3ccc(N4CCO[C@@H](C(C)(C)OC)C4)cn3n2)c2cc(NC(=O)C3CC3)ncc12]

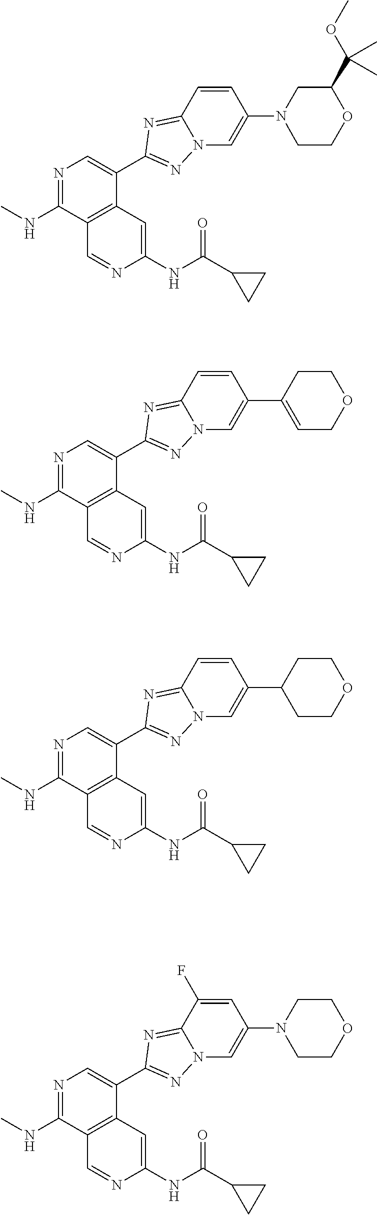

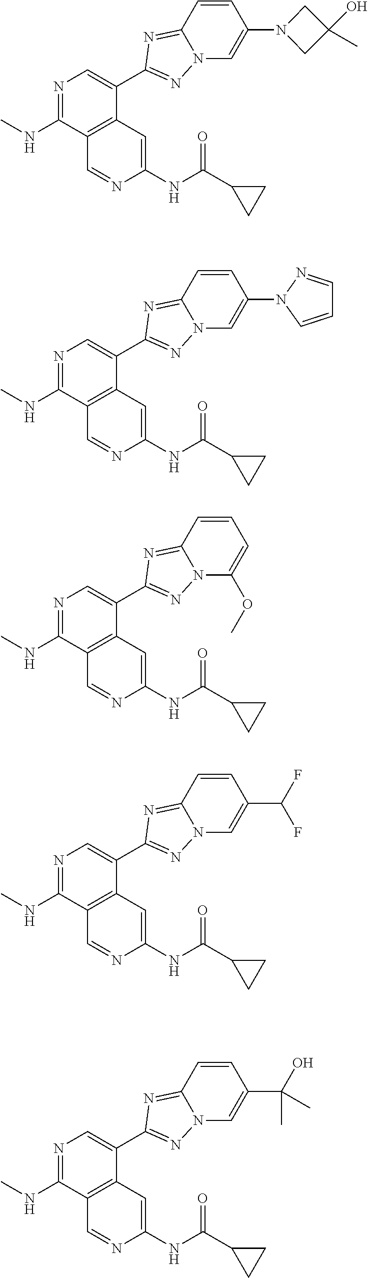

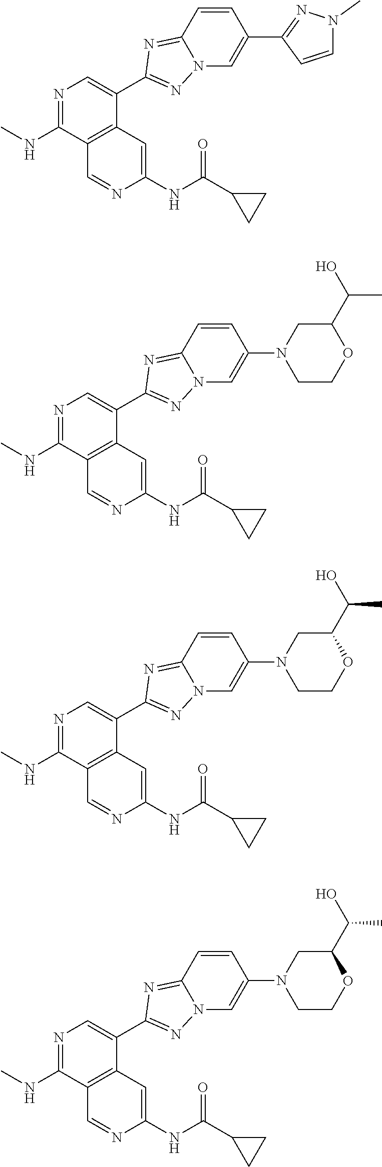

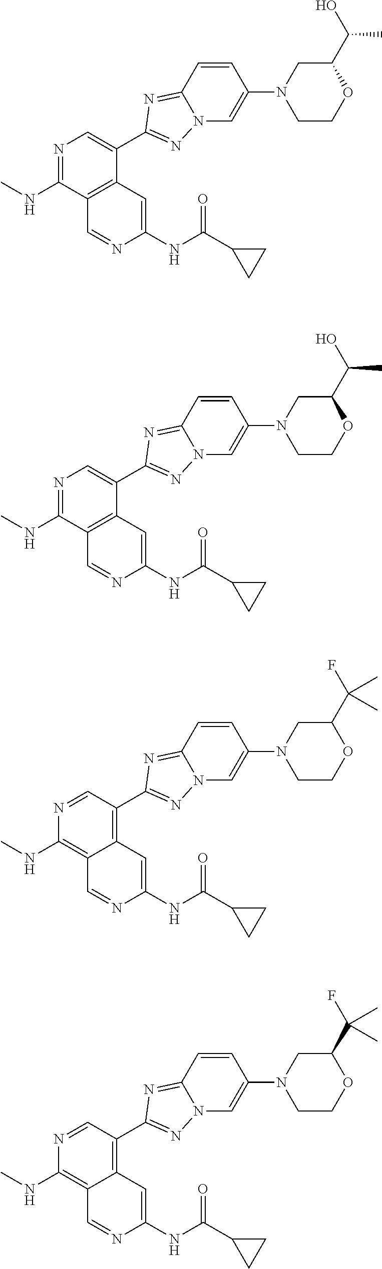

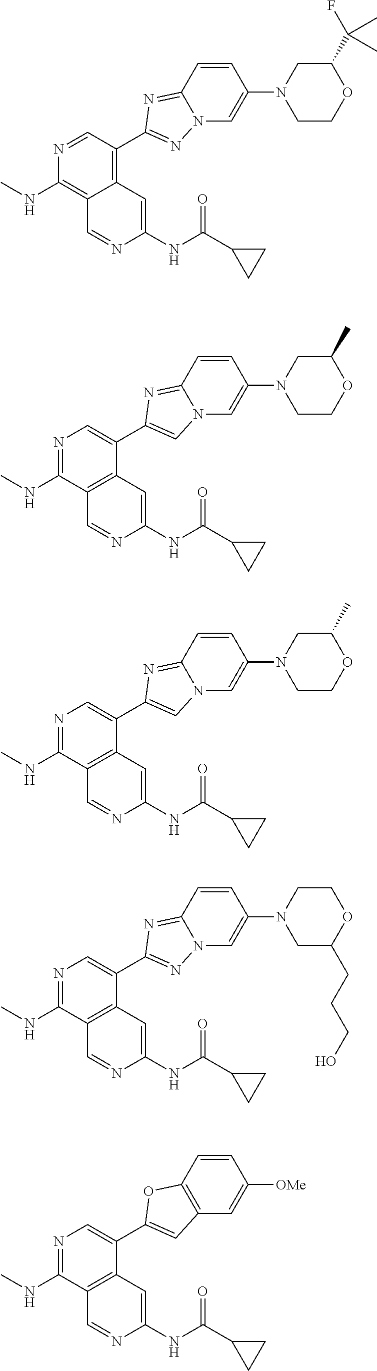

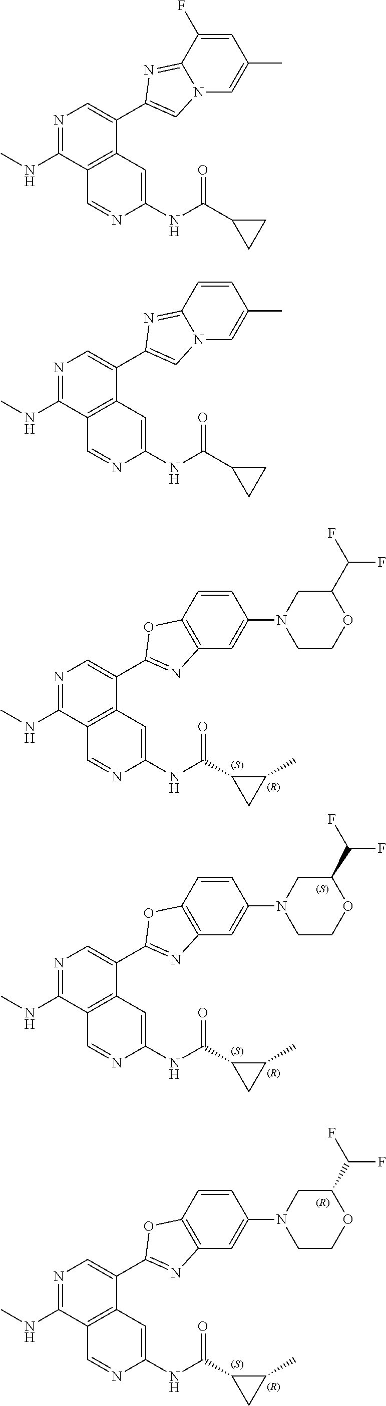

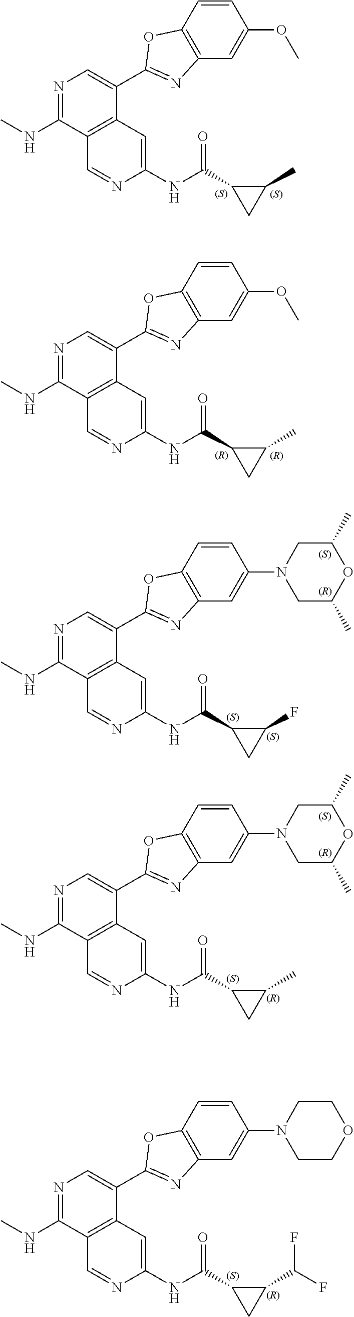

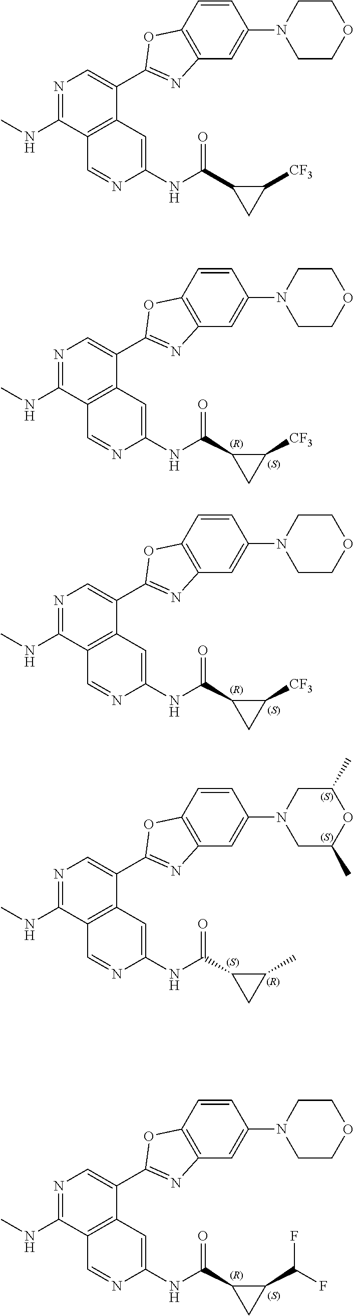

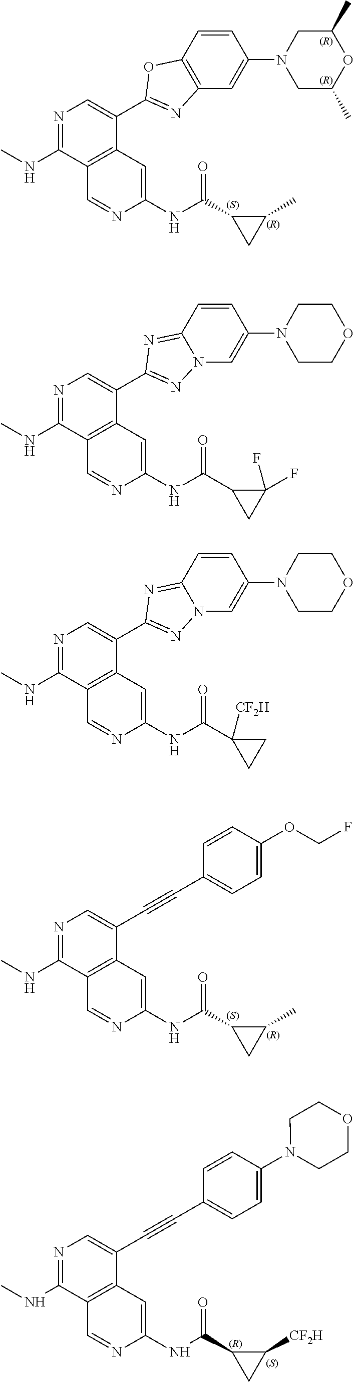

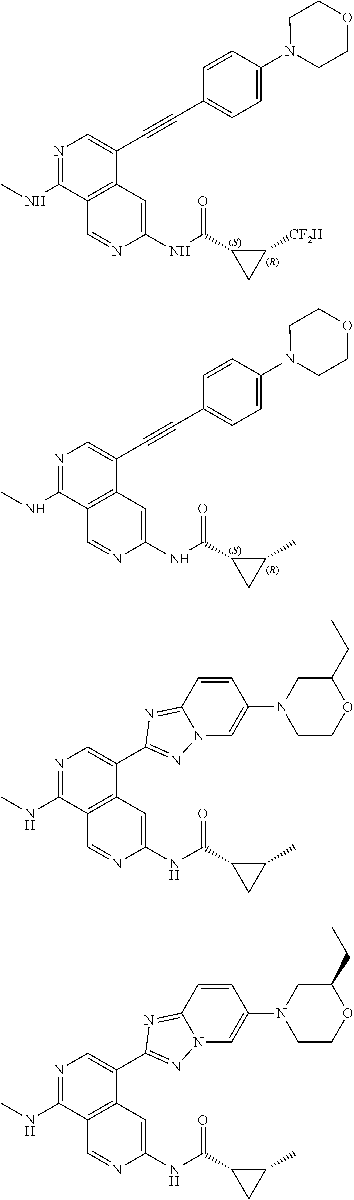

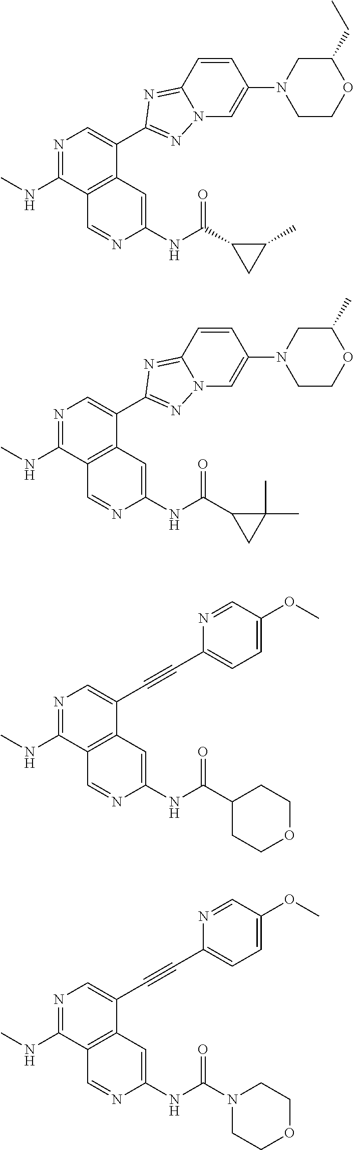

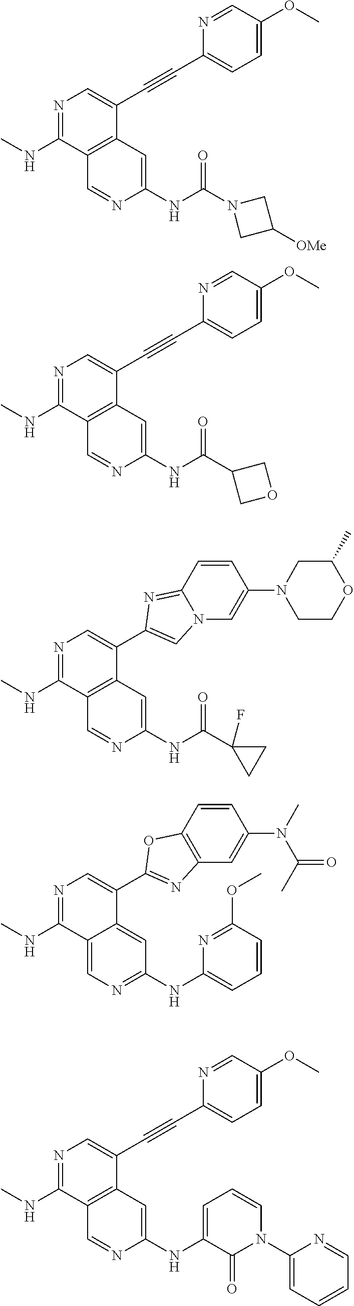

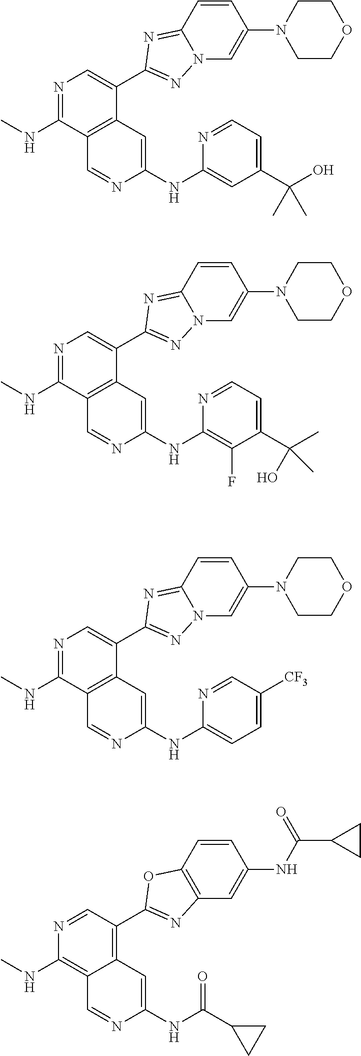 [SMILES: CNc1ncc(-c2nc3ccc(N4CCOCC4)cn3n2)c2cc(Nc3cc(C(C)(C)O)ccn3)ncc12 ; CNc1ncc(-c2nc3ccc(N4CCOCC4)cn3n2)c2cc(Nc3nccc(C(C)(C)O)c3F)ncc12 ; CNc1ncc(-c2nc3ccc(N4CCOCC4)cn3n2)c2cc(Nc3ccc(C(F)(F)F)cn3)ncc12 ; CNc1ncc(-c2nc3cc(NC(=O)C4CC4)ccc3o2)c2cc(NC(=O)C3CC3)ncc12]

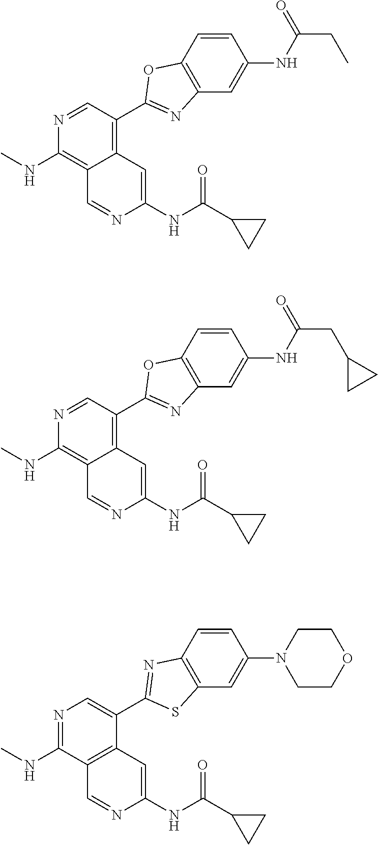

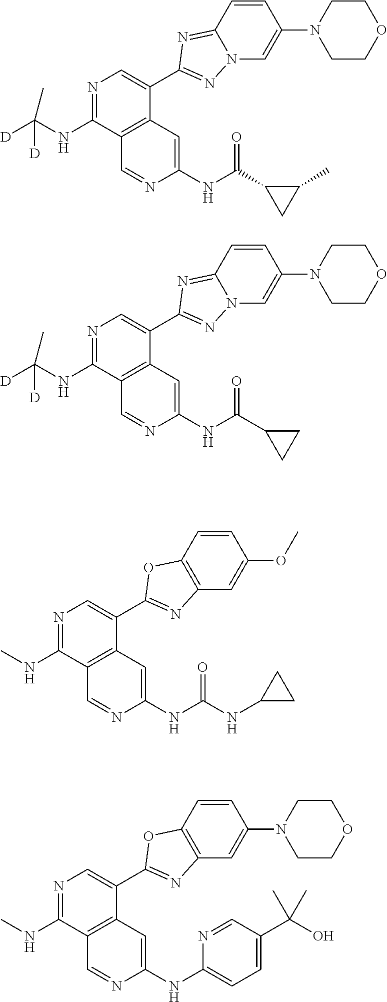

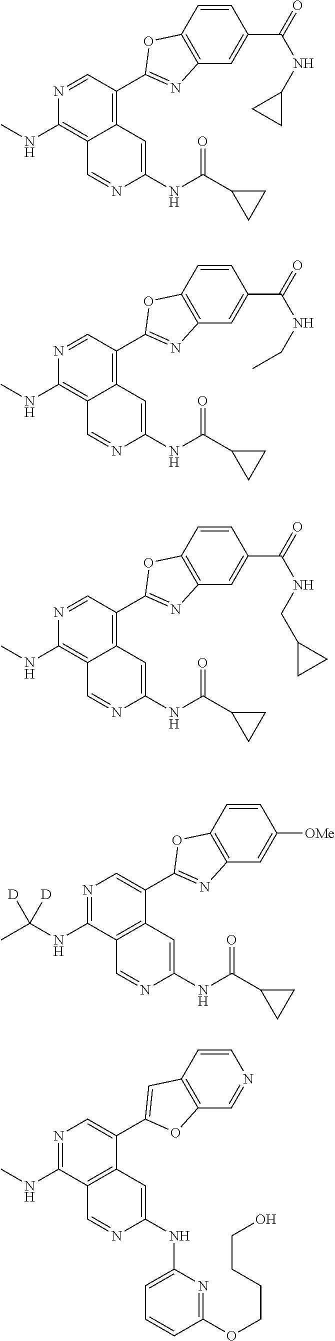 [SMILES: CNc1ncc(-c2nc3cc(C(=O)NC4CC4)ccc3o2)c2cc(NC(=O)C3CC3)ncc12 ; CCNC(=O)c1ccc2oc(-c3cnc(NC)c4cnc(NC(=O)C5CC5)cc34)nc2c1 ; CNc1ncc(-c2nc3cc(C(=O)NCC4CC4)ccc3o2)c2cc(NC(=O)C3CC3)ncc12 ; [2H]C([2H])(C)Nc1ncc(-c2nc3cc(OC)ccc3o2)c2cc(NC(=O)C3CC3)ncc12 ; CNc1ncc(-c2cc3ccncc3o2)c2cc(Nc3cccc(OCCCCO)n3)ncc12]

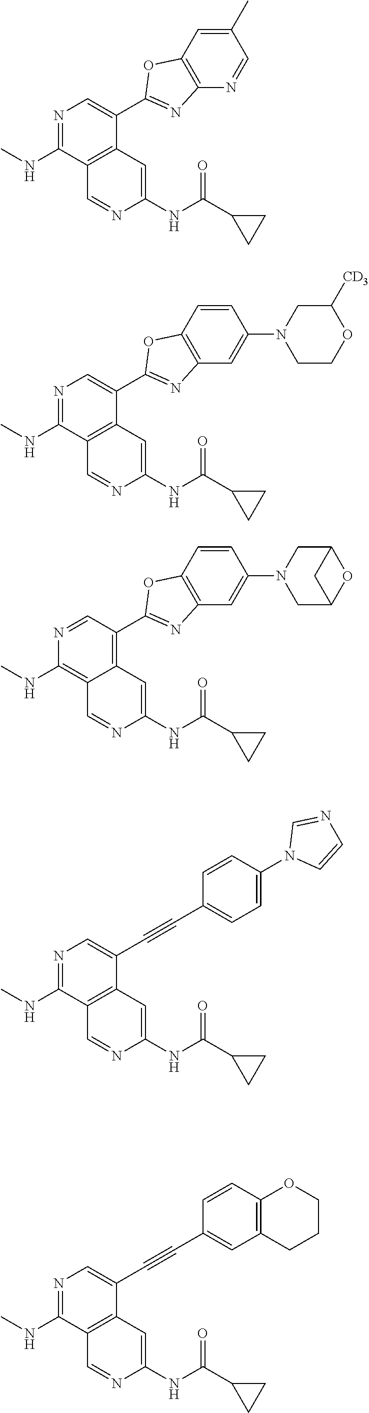

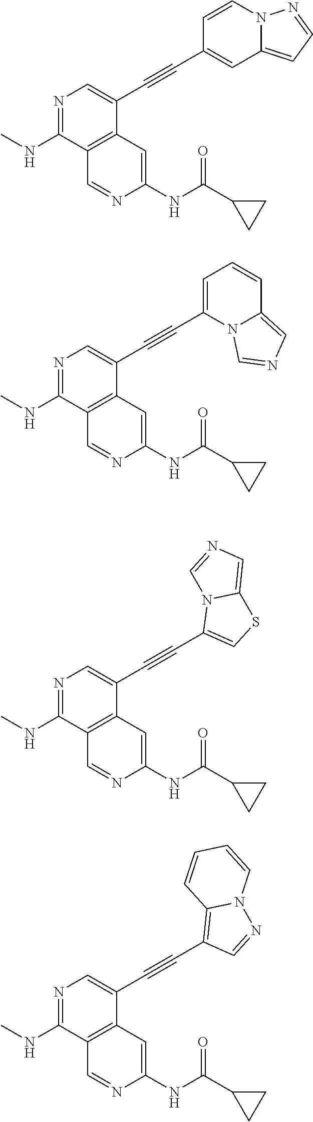

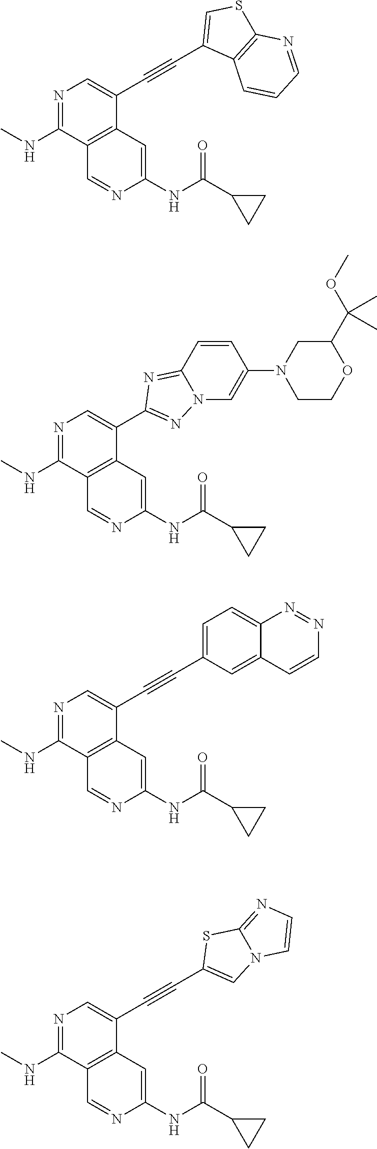

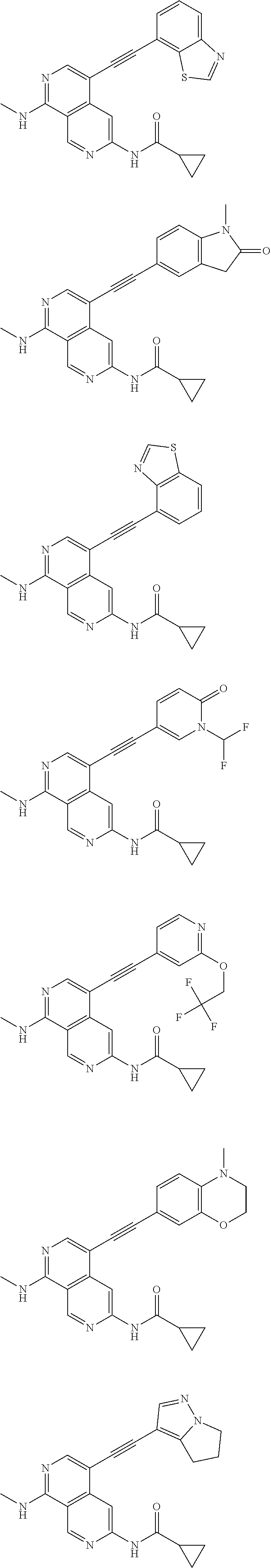

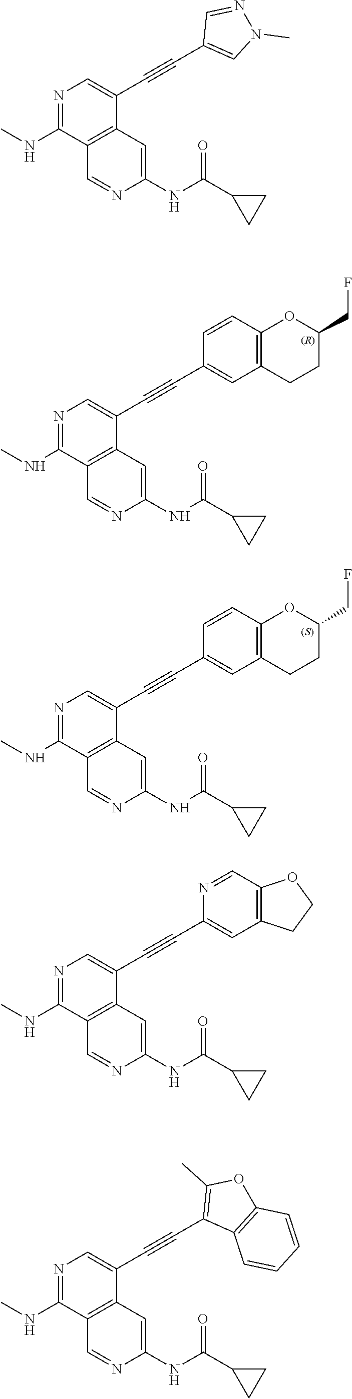

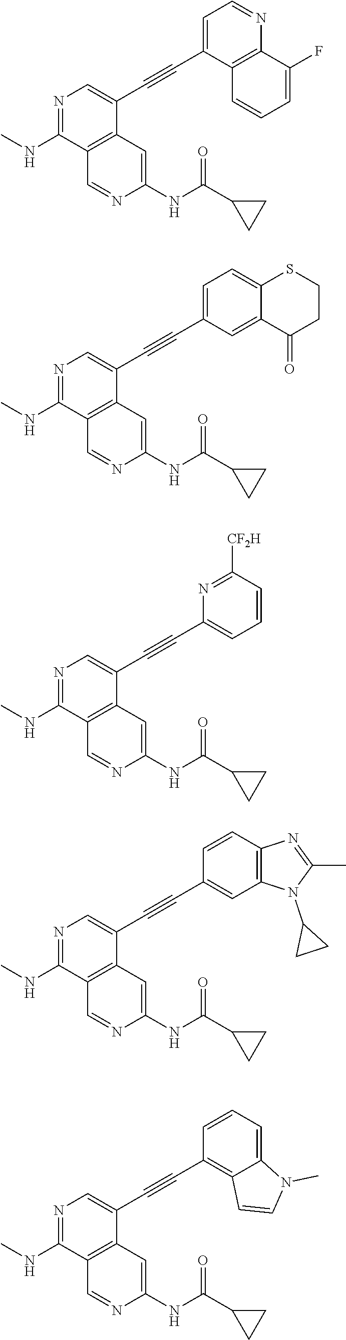

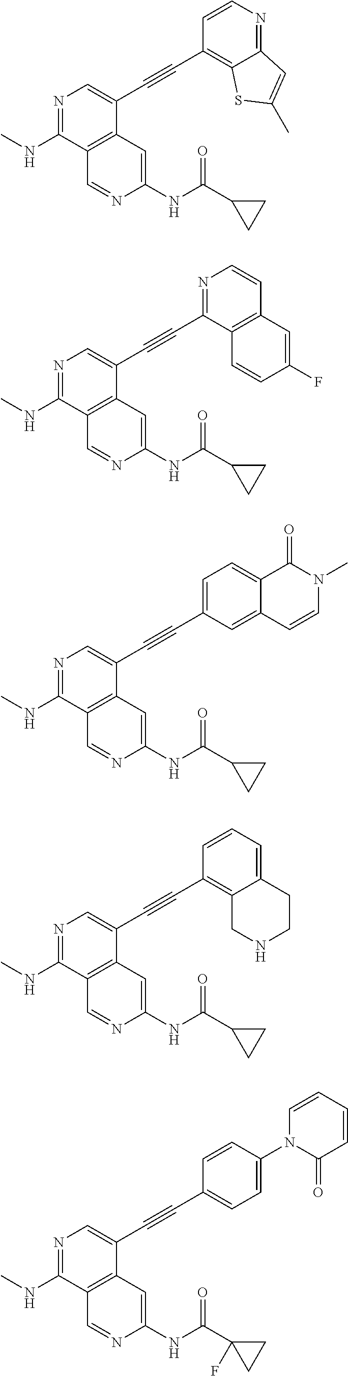

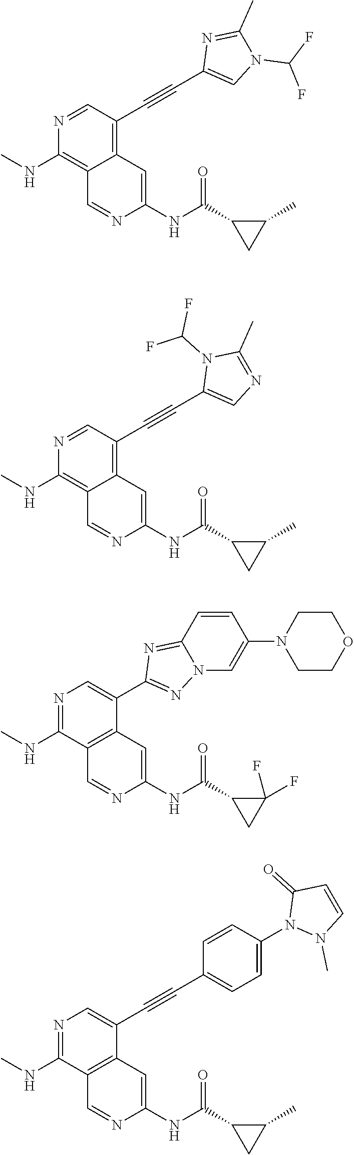

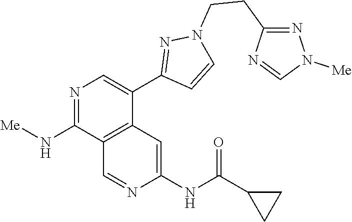 [SMILES: CNc1ncc(-c2ccn(CCc3ncn(C)n3)n2)c2cc(NC(=O)C3CC3)ncc12]

In another aspect, the invention provides pharmaceutical compositions containing one or more compounds of the invention, such as any of the compounds described above.

In another aspect, the invention provides methods of modulating the activity of a kinase by contacting cells containing a kinase with one or more compounds of the invention, such as any of those described above. The compound may inhibit activity of the kinase. The compound may increase activity of the kinase. The kinase may be a JAK family kinase. The kinase may be, LRRK2, NUAK1, or TYK2.

In another aspect, the invention provides methods of treating a condition in a subject by administering to the subject a compound of the invention, such as any of those described above. The condition may be characterized by elevated activity of a kinase. The condition may be characterized by altered activity of a kinase. The kinase may be a JAK family kinase. The kinase may be LRRK2, NUAK1, or TYK2. The condition may be an autoimmune disease, inflammatory disease, bone disease, metabolic disease, neurological or neurodegenerative disease, cancer, cardiovascular disease, allergies, asthma, Alzheimer's disease, Parkinson's disease, skin disorder, eye disease, infectious disease, or hormone-related disease.

In another aspect, the invention provides use of a compound of the invention, such as any of those described above, for making a medicament. In embodiments of the use, the medicament is useful for treating a condition in a subject.

In embodiments of the use the condition is characterize by elevated activity or altered activity of a kinase. In embodiments of the use, the kinase is a JAK family kinase. In embodiments of the use, the kinase is LRRK2, NUAK1, or TYK2.

In embodiments of the use, the condition is an autoimmune disease, inflammatory disease, bone disease, metabolic disease, neurological or neurodegenerative disease, cancer, cardiovascular disease, allergies, asthma, Alzheimer's disease, Parkinson's disease, skin disorder, eye disease, infectious disease, or hormone-related disease.

### BRIEF DESCRIPTION OF THE DRAWING(S)

FIG. 1 provides an overview of JAK and TYK2 signaling pathways.

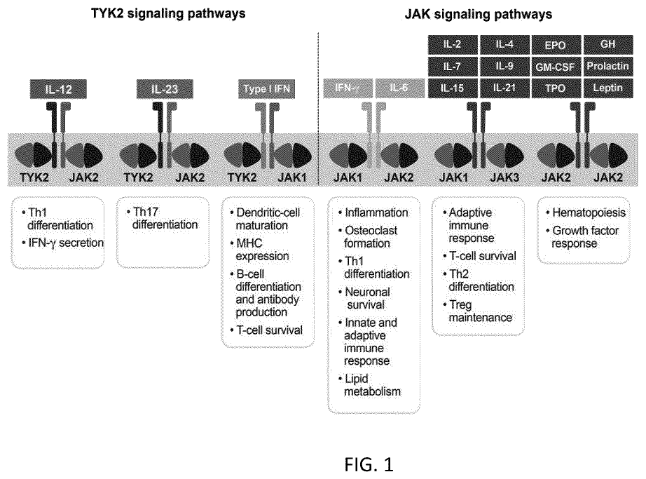

### DETAILED DESCRIPTION

### Chemical Definitions

The expression alkyl refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₃₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₄₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₄₋₃ alkyl”). In some embodiments, an alkyl group has 1 to 2 carbon atoms (“C₃₋₂ alkyl”). In some embodiments, an alkyl group has 1 carbon atom (“C₁ alkyl”). In some embodiments, an alkyl group has 2 to 6 carbon atoms (“C₂ alkyl”). Examples of C₁ alkyl groups include methyl (C₁), ethyl (C₂), n-propyl (C₁), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., from 1 to 5 substituents, 1 to 3 substituents, or 1 substituent. In certain embodiments, the alkyl group is unsubstituted C₁₋₄ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The expression heteroalkyl refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₁₀ alkyl”). In some embodiments, a heteroalkyl group is a saturated group having 1 to 4 carbon atoms and for 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl.”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”).

The expression alkenyl refers to a radical of a straight-chain or branched hydrocarbon group having from 2 to 20 carbon atoms, one or more carbon-carbon double bonds (e.g., 1, 2, 3, or 4 carbon-carbon double bonds) (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms “heteroC₂₋₅ alkenyl”). In some embodiments, a heteroalkenyl group has 2 to 4 carbon atoms, at least one double bond, and 1 or 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroC₂₋₆ alkenyl”). The expression cycloalkyl refers to a saturated or partially unsaturated (for example, a cycloalkenyl group) cyclic group that contains one or more rings, e.g., 2 or 3 rings, and contains from 3 to 14 ring carbon atoms, such as from 3 to 10 (e.g., 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression cycloheteroalkyl or heterocycloalkyl refers to a cycloalkyl group as defined above in which one or more (e.g., 1, 2, or 3) ring carbon atoms have been replaced by an oxygen, nitrogen, silicon, selenium, phosphorus or sulfur atom or a SO group or a SO₂ group. A cycloheteroalkyl or heterocycloalkyl group may have 1 or 2 rings containing from 3 to 10 (e.g., 3, 4, 5, 6 or 7) ring atoms (e.g., C, O, N or S). Cycloheteroalkyl or heterocycloalkyl groups include cycloheteroalkenyl or heterocycloalkenyl groups. The expression cycloheteroalkyl or heterocycloalkyl refers furthermore to groups that are substituted by fluorine, chlorine, bromine or iodine atoms or by OH, ═O, SH, ═S, NH₂, ═NH, N₃ or NO, groups. Examples are a piperidinyl, prolinyl, imidazolidinyl, piperazinyl, morpholinyl, urotro pinyl, pyrrolidinyl, tetrahydrothiophenyl, tetrahydropyranyl, tetrahydrofuryl or 2-pyrazolinyl group and also lactams, lactones, cyclic imides and cyclic anhydrides.

The expression alkylcycloalkyl refers to groups that contain both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings having from 3 to 10 (e.g., 3, 4, 5, 6 or 7) ring carbon atoms, and one or two alkyl or alkynyl groups having 1 or 2 to 6 carbon atoms.

The expression heteroalkylcycloalkyl refers to alkylcycloalkyl groups as defined above in which one or more (e.g., 1, 2 or 3) carbon atoms have been replaced by an oxygen, nitrogen, silicon, selenium, phosphorus or sulfur atom or a SO group or a SO₂ group. A heteroalkylcycloalkyl group preferably contains 1 or 2 rings having from 3 to 10 (e.g., 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalky, alkynytheterocycloalkyl, heteroalkylcycloalkyl, heteroalkylheterocycloalkyl and heteroalkylheterocycloalkenyl, the cyclic groups being saturated or mono-, di- or tri-unsaturated.

The expression aryl refers to an aromatic group that contains one or more rings, e.g., 2 or 3 rings, containing from 6 to 14 ring carbon atoms, such as from 6 to 10 ring carbon atoms. The expression aryl refers furthermore to groups that are substituted by fluorine, chlorine, bromine or iodine atoms or by CH₃, OH, SH, NH₂, N₃ or NO₂ groups. Examples are the phenyl, naphthyl, biphenyl, 2-fluorophenyl, anilinyl, 3-nitrophenyl or 4-hydroxyphenyl group.

The expression heteroaryl refers to an aromatic group that contains one or more rings, e.g., 2 or 3 rings, containing from 5 to 14 ring atoms, such as from 5 to 10 ring atoms, and contains one or more (e.g., 1, 2, 3 or 4) oxygen, nitrogen, phosphorus or sulfur ring atoms. The expression heteroaryl refers furthermore to groups that are substituted by fluorine, chlorine, bromine or iodine atoms or by CH₃, OH, SH, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, aryl-alkyl, arylalkenyl, aryl alkynyl, arylcycloalkyl, arycycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (e.g., 1, 2, 3 or 4) carbon atoms have been replaced by an oxygen, nitrogen, silicon, selenium, phosphorus, boron or sulfur atom, that is to say to groups containing both aryl or heteroaryl, respectively, and also alkyl, alkenyl, alkynyl and/or heteroalkyl and/or cycloalkyl and/or heterocycloalkyl groups in accordance with the above definitions. A heteroaralkyl group preferably contains one or two aromatic ring systems containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms.

Examples are arylheteroalkyl, arylheterocycloalkyl, arylheterocycloalkenyl, arylalkyl heterocycloalkyl, arylalkenylheterocycloalkyl, arylalkynylheterocycloalkyl, arylalkylhetero cycloalkenyl, heteroarylalkyl, heteroarylalkenyl, heteroarylalkynyl, heteroarylheteroalkyl, heteroarylcycloalkyl, heteroarylcycloalkenyl, heteroarylheterocycloalkyl, hetero arylheterocycloalkenyl, heteroarylalkylcyclo alkyl, heteroarylalkylheterocycloalkenyl, hetero arylheteroalkylcycloalkyl, heteroarylheteroalkylcycloalkenyl and heteroarylheteroalkylhetero cycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

As stated above, the expressions cycloalkyl, cycloheteroalkyl, heterocycloalkyl, alkylcycloalkyl, heteroalkylcycloalkyl, aryl, heteroaryl, aralkyl and heteroaralkyl also refer to groups that are substituted by fluorine, chlorine, bromine or iodine atoms or by CH₃, OH, ═O, SH, ═S, ═NH, N₃ or NO₂ groups.

The expression carbocyclyl or carbocyclic refers to a radical of a non-aromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms 10 (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (G), cyclooctenyl (G), bicyclo[2.2.1]heptanyl (C₇), bicyclo[2.2.2]octanyl (G), and the like. Exemplary C₃₋₁₀ carbocyclyl groups include, without 20 limitation, the aforementioned G-s carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4,5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted. (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms “C₅₋₆ cycloalkyl”). In some embodiments, a cycloalkyl group has 5 to 10 ring carbon atoms “C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (CO. Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The expression heterocyclyl or heterocyclic refers to a radical of a 3- to 14-membered non-aromatic ring system having ring carbon atoms and 1 to 4 ring heteroatoms, wherein each heteroatom is independently selected from nitrogen, oxygen, sulfur, boron, phosphorus, and silicon (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups 5 containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary, 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The expression optionally substituted means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Heteroatoms, such as nitrogen, may have substituents, such as any suitable substituent described herein which satisfies the valencies of the heteroatoms and results in the formation of a stable moiety.

For example and without limitation, optional substituents include fluorine, chlorine, bromine, and iodine atoms and CF₃, CN, OH, ═O, SH, ═S, NH₂, ═NH, N₃ and NO₂ groups. Optional substituents also include C₁-C₁₀ alkyl, C₂-C₁₀ alkenyl, C₁-C₁₀ heteroalkyl, C₃-C₁₆ cycloalkyl, C₂-C₁₇ heterocycloalkyl, C₁-C₂₀ alkylcycloalkyl, C₂-C₁₉ heteroalkylcycloalkyl, C₆-C₁₈ aryl, C₁₋₁₇ heteroaryl, C₇-C₂₀ aralkyl or C₂-C₁₉ heteroaralkyl, C₁-C₆ alkyl, C₂-C₆ alkyl, C₁-C₆ heteroalkyl, C₃-C₁₀ cycloalkyl, C₂-C₉ heterocycloalkyl, C₇-C₁₂ alkylcycloalkyl, heteroalkylcycloalkyl, C₆-C₁₀ aryl. C₁-C₉ heteroaryl, C₇-C₁₂ aralkyl, C₂-C₁₁ heteroaralkyl, and C₁-C₁₀ haloalkyl groups.

Exemplary substituents are F, C₁, Br, OH, SH, ═O, NH₂, amino, C₁₋₄ alkyl, C₁₋₄ heteroalkyl cyclopropyl, SF₅, NO, NO₂.

Other exemplary substituents are F, Cl, Br, OH, SH, ═O, NH₂, C₁₋₄ alkyl (e.g. methyl, ethyl, t-butyl NMe₂, CONH₂, CH₂NMe₂, NHSO₂Me, C(CH₃)₂CN, COMe, OMe, SMe, COOMe, COOEt, CH₂COOH, OCH₂COOH, COOH, SOMe, SO₂Me, cyclopropyl, SO₂, H₂, SO₂NHMe, SO₂CH₂CH₂OH, NHCH₂CH₂OH, CH₂CH₂OCH₃, SF₅, SO₂NMe₂, NO, NO₂, OCF₃, SO₂CF₃, CN or CF₃.

Other exemplary substituents are F, Cl, Br, Me, OMe, CN or CF₃.

The term halogen preferably refers to F, Cl, Br or I.

According to certain embodiments, all alkyl, alkenyl, alkynyl, heteroalkyl, aryl, heteroaryl, cycloalkyl, heterocycloalkyl, alkylcycloalkyl, heteroalkylcycloalkyl, aralkyl and heteroaralkyl groups described herein may optionally be substituted.

When an aryl, heteroaryl, cycloalkyl, alkycyloalkyl, heteroalkylcycloalkyl, heterocycloalkyl, aralkyl or heteroaralkyl group contains more than one ring, these rings may be bonded to each other via a single or double bond or these rings may be annulated.

Other optional substituents include, but are not limited to, halogen, —CN, —NO₂, —N₃, —SO₂H, —SO₃H, —OH, —OR^{aa}, —ON(R^{bb})₂, —N(R^{bb})²), —N(R^{bb})₃⁺X⁻, —N(OR^{cc})R^{bb}, —SH, —SR^{aa}, —SSR^{CC}, —C(O)R^{aa}, —CO₂H, —CHO, —C(OR^{cc})₂, —CO₂R^{aa}, —OC(O)R^{aa}, —OCO₂R^{aa}, —C(O)N(R^{bb})₂, —C(O)N(R^{aa})(R^{bb}), —OC(O)N(R^{bb})₂, —NR^{bb}C(O)R^{aa}, —NR^{bb}CO₂R^{aa}, —NR^{bb}C(O)N(R^{bb})₂, —C(NR^{bb})R^{aa}, —C(NR^{bb})OR^{aa}, —OC(NR^{bb})R^{aa}, —OC(NR^{bb})OR^{aa}, —C(NR^{bb})N(R^{bb})², —OC(NR^{bb})N(R^{bb})₂, —NR^{bb}C(NR^{bb})N(R^{bb})₂, —C(O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(O)R^{aa}, e.g. —S(O)R^{aa}, —OS(O)R^{aa}, —Si(R^{aa})₃, —OSi(R^{aa})₃—C(S)N(R^{bb})₂, —C(O)SR^{aa}, —C(S)SR^{aa}, —SC(S)SR^{aa}, —SC(O)SR^{aa}, —OC(O)SR^{aa}, —SC(O)OR^{aa}, —SC(O)R^{aa}, —P(O)₂R^{aa}, —OP(O)₂R^{aa}, —P(O)(R^{aa})₂, —OP(O)(R^{aa})₂, —OP(O)(OR^{cc})₂, —P(O)₂N(R^{bb)}₂, —OP(O)₂N(R^{bb})₂, —P(O)(NR^{bb})₂, —OP(O)(NR^{bb})₂, —NR^{bb}P(O)(OR^{cc})₂, —NR^{bb}P(O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, wherein each alkyl, alkenyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})², ═NNR^{bb}C(O)R^{aa}, ═NNR^{bb}C(O)OR^{aa}, ═NNR^{bb}S(O)₂R^{aa}, —NR^{bb}, or ═NOR^{cc}; in which:
each instance of R^{aa} is, independently, selected from C₁₋₁₀ alkyl, C₁₋₁₀ heteroalkyl, C₁₋₁₀ haloalkyl, C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered cycloalkyl, 3-14 membered cycloheteroalkyl, 3-14 membered heterocyclyl or 5-14 membered heteroaryl ring, wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 groups;
each instance of R^{bb} is, independently, selected from hydrogen, —OH, —OR^{aa}, —N(R^{aa})₂, —CN, —C(O)R^{aa}, —C(O)N(R^{cc})₂, —CO₂R^{aa}, —SO₂R^{aa}, —C(NR^{cc})OR^{aa}, —C(NR^{cc})N(R^{cc})₂, —SO₂N(R^{cc})₂, —SO₂R^{cc}, —SO₂OR^{cc}, —C(S)N(R^{cc})₂, —C(O)SR^{cc}, —C(S)SR^{cc}, —P(O)₂R^{aa}, —P(O)(R^{aa})₂, —P(O)₂N(R^{cc})₂, —P(O)(NR^{cc})₂, C₁₋₁₀ alkyl, C₁₋₁₀ heteroalkyl, C₁₋₁₀ haloalkyl, C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring; wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, carbocyclyl, heterocyclyl, aryl; and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(Rn); CX˜, —N(OR^{ee})R^{ff}, —SH, —SR^{ee}, —SSR^{ee}, —C(O)R^{ee}, —CO₂H, —CO₂R^{ee}, —OC(O)R^{ee}, —OCO₂R^{ee}, —C(O)N(R^{ff})₂, —OC(O)N(R^{ff})₂, —NR^{ff}C(O)R^{ee}, —NR^{ff}CO₂R^{ee}, —NR^{ff}C(O)N(R^{ff})₂, —C(NR^{ff})OR^{ee}, —OC(NR^{ff})R^{ee}, —OC(NR^{ff})OR^{ee}, —C(NR^{ff})N(R^{ff})₂, —OC(NR^{ff})N(R^{ff})₂, —NR^{ff}C(NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(O)R^{ee}, e.g., —S(O)R^{ee}, —Si(R^{ee})₃, —OSi(R^{ee})₃, —C(S)N(R^{ff})₂, —C(O)SR^{ee}, —C(S)SR^{ee}, —SC(S)SR^{ee}, —P(O)₂R^{ee}, —P(O)(R^{ee})₂, —OP(O)(R^{ee})², —OP(O)(OR^{ee})₂, C₁₋₆ alkyl, C₁₋₆ heteroalkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, wherein each alkyl, alkenyl, carbocyclyl, heterocyclyl, aryl, and heteroaryl is independently substituted with 0, 1, or 5 R^{gg} groups, or two geminal R^{dd} substituents can be joined to form ═O or ═S;
each instance of R^{ee} is, independently, selected from C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, wherein each alkyl, alkenyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, or two R^{ff} groups are joined to form a 3-14 membered heterocyclyl or 5-14 membered heteroaryl ring, wherein each alkyl, alkenyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, alkyl)₃⁺X⁻, —NH(C₁₋₆ alkyl)₂⁺X⁻, —NH₂(C₁₋₆ alkyl)⁺X⁻-MR⁺X⁻, N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(O)(C₁₋₆ alkyl), —CO₂H, —CO₂(C₁₋₆ alkyl), —OC(O)(C₁₋₆ alkyl), —OCO₂(C₁₋₆ alkyl), —C(O)NH₂, —C(O)N(C₁₋₆ alkyl)₂, OC(O)NH(C₁₋₆ alkyl), —NHC(O)(C₁₋₆ alkyl), —N(C₁₋₆ alkyl)C(O)(C₁₋₆ alkyl), —NHCO₂(C₁₋₆ alkyl), —NHC(O)N(C₁₋₆ alkyl)₂, —NHC(O)NH(C₁₋₆ alkyl), —NHC(O)NH₂, —C(NH)O(C₁₋₆alkyl), —OC(NH)(C₁₋₆ alkyl), —OC(NH)OC₁₋₆ alkyl, —C(NH)N(C₁₋₆ alkyl)₂, —C(NH)NH(C₁₋₆ alkyl), —C(NH)NH₂, —OC(NH)N(C₁₋₆ alkyl)₂, —OC(NH)NH(C₁₋₆ alkyl), —OC(NH)NH₂, —NHC(NH)N(C₁₋₆ alkyl)₂, —NHC(NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(S)N(C₁₋₆ alkyl)₂, C(S)NH(C₁₋₆ alkyl), C(S)NH₂, —C(O)S(C₁₋₆ alkyl), —C(S)SC₁₋₆ alkyl, —SC(S)SC₁₋₆ alkyl, —P(O)₂(C₁₋₆ alkyl), —P(O)(C₁₋₆ alkyl)₂, —OP(O)(C₁₋₆ alkyl)₂, —OP(O)(OC₁₋₆alkyl)₂, C₁₋₆ alkyl, C₁₋₆ haloalkyl, C₂₋₆ alkenyl, C₃₋₁₀ carbocyclyl, C₃₋₁₀ aryl, 3-10 membered heterocyclyl, 5-10 membered heteroaryl; or two geminal R^{gg} substituents can be joined to form ═O or ═S; wherein X⁻ is a counterion.

### Compounds

In certain aspects, the invention provides compounds of formula (I):

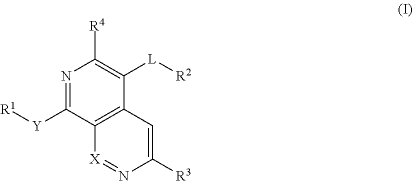

and pharmaceutically acceptable salts thereof,
wherein:
X is CH or N;
Y is CH₂, S, or NH;
L is a single bond, double bond, triple bond substituted or unsubstituted alkyl, heteroalkyl, alkoxy, heteroalkoxy, cycloalkyl, heterocycloalkyl, haloalkyl, halocycloalkyl, aryl; heteroaryl; —C(O)NH—, —NHC(O)—, O, NH, or S;
R¹ is alkyl, cycloalkyl, haloalkyl, halocycloalkyl, aryl, heteroaryl, methyl, CD₃, or H;
R² is H, halo, alkyl, branched alkyl, alkenyl, alkynyl, haloalkyl, alkoxy, cycloalkoxy, haloalkoxy, nitro, cyano, or a 5 or 6 membered substituted or unsubstituted aryl, or monocyclic or bicyclic heteroaryl ring optionally containing one or more heteroatoms independently selected from O, S, and N,
wherein the substitutions on the said 5 or 6 membered aryl or heteroaryl rings are: H, halo, alkyl, branched alkyl, alkenyl, alkynyl, haloalkyl, alkoxy, cycloalkoxy, haloalkoxy, thioalkyl, nitro; cyano, —CH₂-cycloalkyl, —CF₂-cycloalkyl, —CH(CH₃)-cycloalkyl, —CH₂-aryl, —CF₃, —CF₂-aryl, —CH(—CH₃)-aryl, C(═O)-alkyl, —C(═O)cycloalkyl, —C(═O)—NH-alkyl, —C(═O)NH₂, hydroxy, —COOH (and ester thereof), sulfonyl, alkylsulfonyl, aryl sulfonyl, sulfonamide, amino, 3-6 membered cycloalkyl or heterocycloalkyl, 3-6 membered aryl or heteroaryl, any of which may have one or more substituents;
R³ is H, halo, alkyl, branched alkyl, alkenyl, alkynyl, cycloalkyl, haloalkyl, alkoxy, cycloalkoxy, haloalkoxy, nitro, cyano, aminoalkyl, aminocycloalkyl, aminoheterocycloalkyl, —NH-aryl, —NH-heteroaryl, —NH-phenyl, —NH₂, —NH—CH—CF₃, substituted or unsubstituted C(═O)cycloalkyl, substituted or unsubstituted —NH—C(═O)cycloalkyl, substituted or unsubstituted —NH—C(═O)cycloalkyl, substituted or unsubstituted aminoalkylaryl; and R⁴ is selected from a group consisting of: H, halo, alkyl, branched alkyl, alkenyl, alkynyl, cycloalkyl, haloalkyl, alkoxy, cycloalkoxy, haloalkoxy, nitro, cyano, and alkylhydroxyl.

In certain embodiments, Y is NH.

In certain embodiments, R₁ is methyl or ethyl.

In certain embodiments, L is a single bond.

In certain embodiments, X is CH.

In certain embodiments, X is N.

In certain embodiments, R³ is:

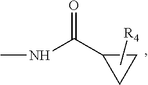

wherein R⁴ is H, halo, alkyl, branched alkyl, alkenyl, alkenyl; cycloalkyl, spirocycloalkyl, haloalkyl, alkoxy, cycloalkoxy, haloalkoxy, nitro, or cyano.

In certain embodiments, R³ is:

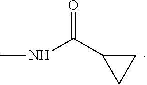

In certain embodiments, R² is:

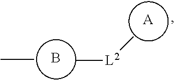

wherein L² is substituted or unsubstituted alkyl; heteroalkyl, alkoxy, heteroalkoxy, cycloalkyl, heterocycloalkyl, haloalkyl, halocycloalkyl, aryl, heteroaryl, or a bond linking the groups;

A or B are independently 5 or 6 membered substituted or unsubstituted aryl or heteroaryl ring optionally containing one or more heteroatoms independently selected from O, S, and N, wherein the substitutions on the said 5 or 6 membered aryl or heteroaryl ring are: H, halo; alkyl, branched alkyl, alkenyl, alkynyl, haloalkyl, alkoxy, cycloalkoxy, haloalkoxy, nitro, cyano, —CH₂-cycloalkyl, —CF₂-cycloalkyl, —CH(CH₃)-cycloalkyl, —CH₇-aryl, —CF₂-aryl, —CH(—CH₃)-aryl, C(═O)-alkyl, —C(═O)cycloalkyl, —C(═O)—NH-alkyl, —C(═)NH₂, hydroxy, —COOH (and ester thereof), alkylsulfonyl, arylsulfonyl, sulfonamide, amino, 3-6 membered cycloalkyl or heterocycloalkyl, 3-6 membered aryl or heteroaryl, any of which may have one or more substituents.

In certain embodiments, R² is phenyl.

In certain embodiments, R² is:

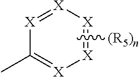

wherein each X is independently N or CH;
R⁵ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, and substituted or unsubstituted heteroaryl, and n is 0, 1, 2, or 3.

In certain embodiments, R² is:

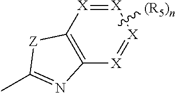

wherein Z is O or S, each X is independently N or CH;
R⁵ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NHR⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O) R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, substituted or unsubstituted heteroaryl; and n is 0, 1, 2 or 3.

In certain embodiments, R² is:

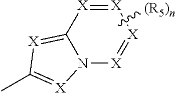

wherein each X is independently N or CH; R₅ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroarkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O) R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl; substituted or unsubstituted bridgedbicycloheteroalkyl, substituted or unsubstituted spirobicycloheteroalkyl, substituted or unsubstituted aryl, substituted or unsubstituted heteroaryl; and n is 0, 1, 2 or 3.

In certain embodiments, L-R² is:

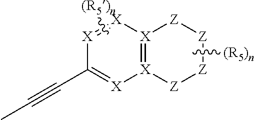

wherein each X is independently N or CH;
Z is independently O or NR⁶

R⁵ and R^{5′} is independently H, halogen, hydroxyl, —CN, alkyl; haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶; —S(O)NHR⁶, —S(O)(NH)R⁶, —S(O)(NMe)R⁶, —(CH₂)ₙS(O)R⁶, —(CH₂)ₙOR⁶, —P(O) R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted-bridgedbicycloheteroalkyl, substituted or unsubstituted spirobicycloheteroalkyl, substituted or unsubstituted aryl, or substituted or unsubstituted heteroaryl; and n=1, 2 or 3.

In certain embodiments, L-R² is:

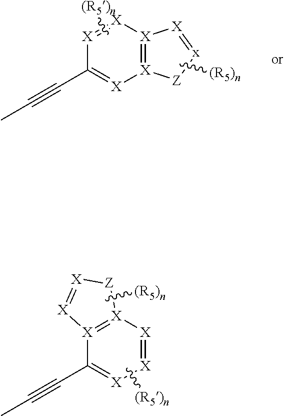

wherein Z is N or O,
each X is independently N or CH;
R₅ and R₅′ is independently H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted-bridgedbicycloheteroalkyl, substituted or unsubstituted spirobicycloheteroalkyl, substituted or unsubstituted aryl, or substituted or unsubstituted heteroaryl; and
n is 0, 1, 2, or 3.

In certain embodiments, L-R² is:

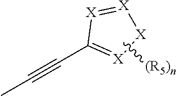

wherein R₅ is selected from a group consisting of: H, halogen, hydroxyl, —CN, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O) R⁶R^{6′} where R⁶ and R^{6′} is independently alkyl, branched alkyl, haloalkyl, substituted or unsubstituted, cycloalkyl, substituted or un substituted cycloheteroalkyl, substituted or un substituted fusedbicycloheteroalkyl, substituted or unsubstituted bridgedbicycloheteroalkyl, substituted or unsubstituted spirobicycloheteroalkyl, substituted or unsubstituted aryl, and substituted or unsubstituted heteroaryl; and n is 0, 1, 2, or 3.

In another preferred embodiment, the compound of formula (I) is selected from the group consisting of:

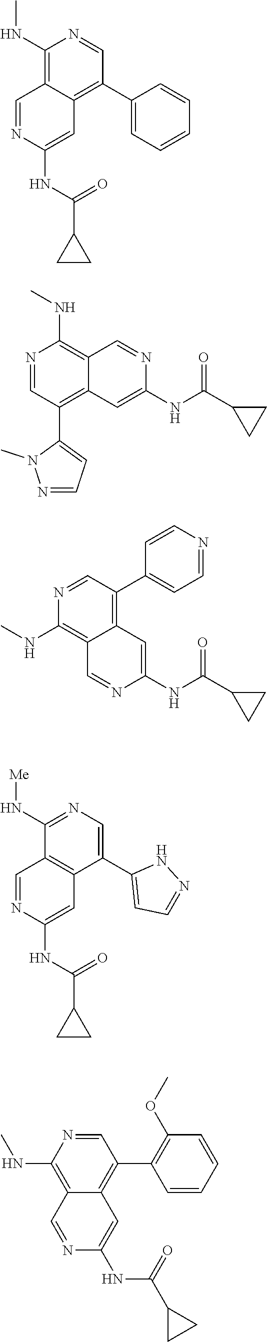

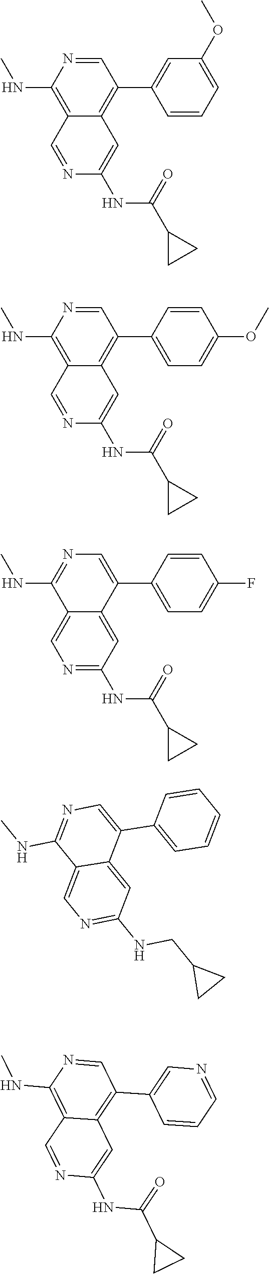

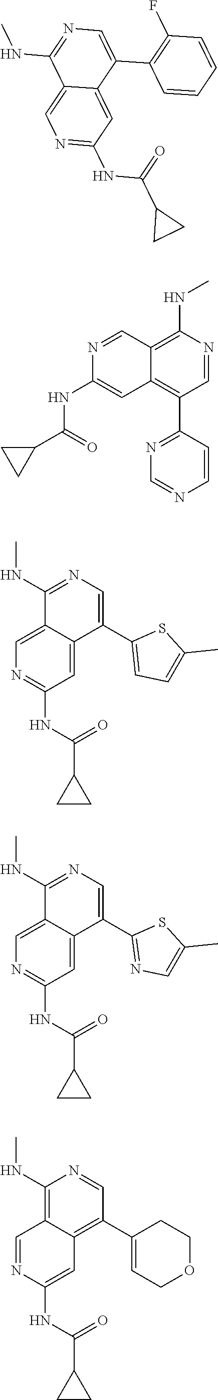 [SMILES: CNc1ncc(-c2ccccc2F)c2cc(NC(=O)C3CC3)ncc12 ; CNc1ncc(-c2ccncn2)c2cc(NC(=O)C3CC3)ncc12 ; CNc1ncc(-c2ccc(C)s2)c2cc(NC(=O)C3CC3)ncc12 ; CNc1ncc(-c2ncc(C)s2)c2cc(NC(=O)C3CC3)ncc12 ; CNc1ncc(C2=CCOCC2)c2cc(NC(=O)C3CC3)ncc12]

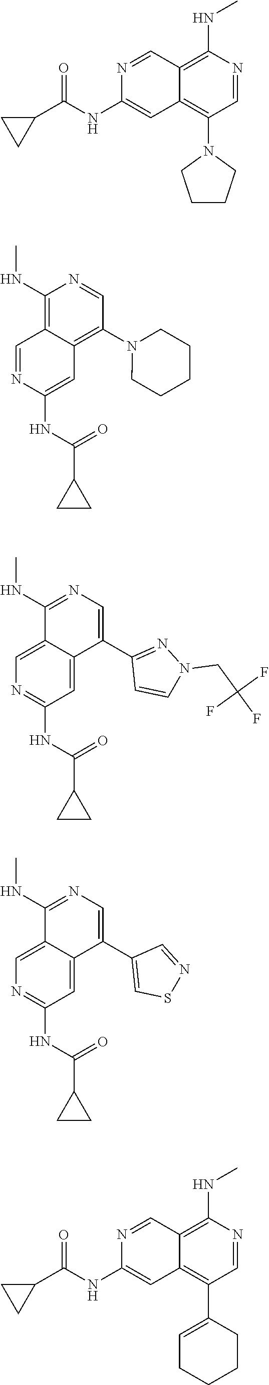 [SMILES: CNc1ncc(N2CCCC2)c2cc(NC(=O)C3CC3)ncc12 ; CNc1ncc(N2CCCCC2)c2cc(NC(=O)C3CC3)ncc12 ; CNc1ncc(-c2ccn(CC(F)(F)F)n2)c2cc(NC(=O)C3CC3)ncc12 ; CNc1ncc(-c2cnsc2)c2cc(NC(=O)C3CC3)ncc12 ; CNc1ncc(C2=CCCCC2)c2cc(NC(=O)C3CC3)ncc12]

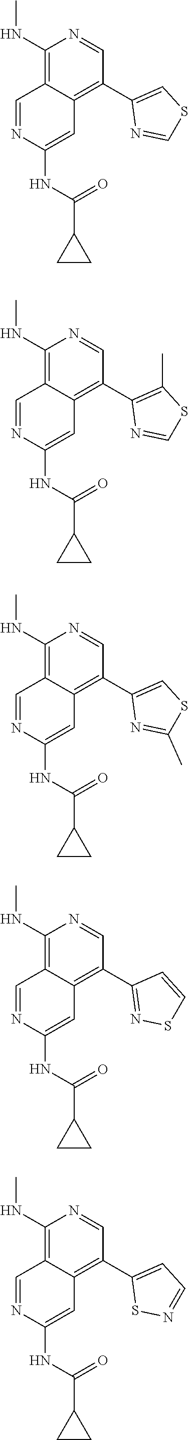 [SMILES: CNc1ncc(-c2cscn2)c2cc(NC(=O)C3CC3)ncc12 ; CNc1ncc(-c2ncsc2C)c2cc(NC(=O)C3CC3)ncc12 ; CNc1ncc(-c2csc(C)n2)c2cc(NC(=O)C3CC3)ncc12 ; CNc1ncc(-c2ccsn2)c2cc(NC(=O)C3CC3)ncc12 ; CNc1ncc(-c2ccns2)c2cc(NC(=O)C3CC3)ncc12]

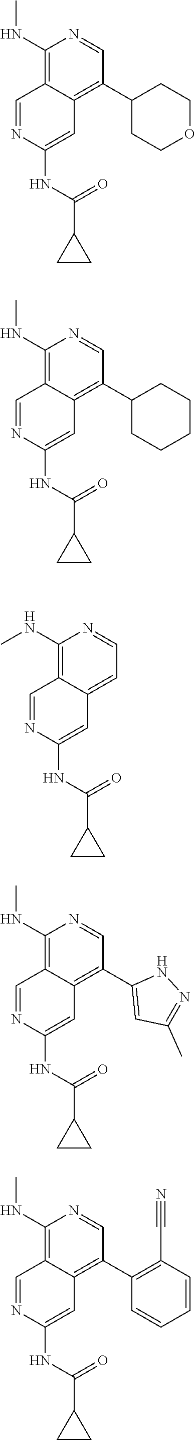 [SMILES: CNc1ncc(C2CCOCC2)c2cc(NC(=O)C3CC3)ncc12 ; CNc1ncc(C2CCCCC2)c2cc(NC(=O)C3CC3)ncc12 ; CNc1nccc2cc(NC(=O)C3CC3)ncc12 ; CNc1ncc(-c2cc(C)n[nH]2)c2cc(NC(=O)C3CC3)ncc12 ; CNc1ncc(-c2ccccc2C#N)c2cc(NC(=O)C3CC3)ncc12]

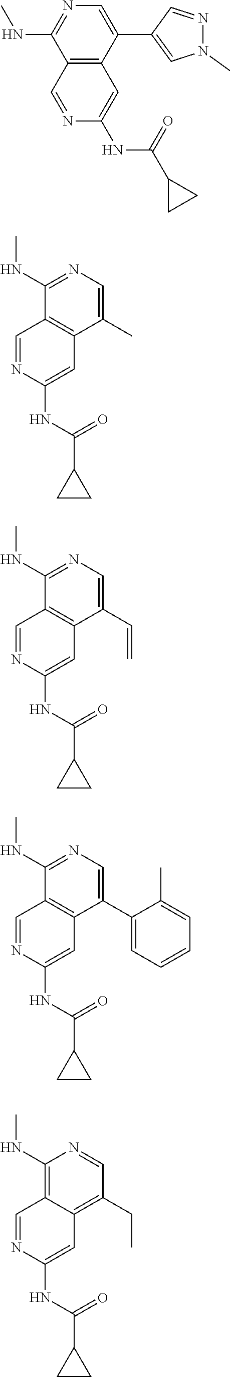

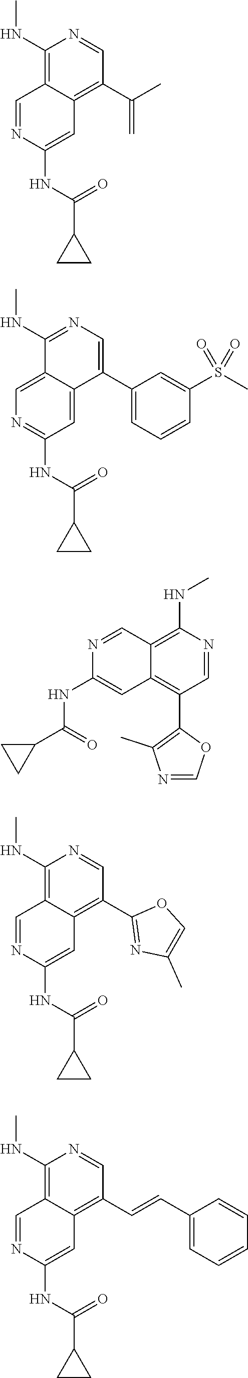

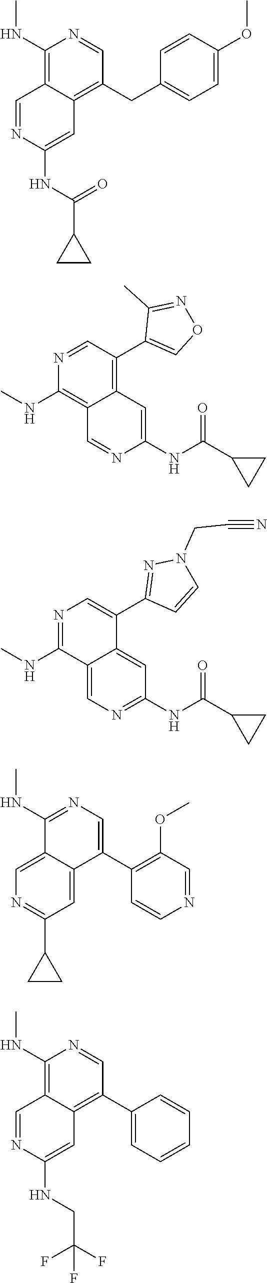

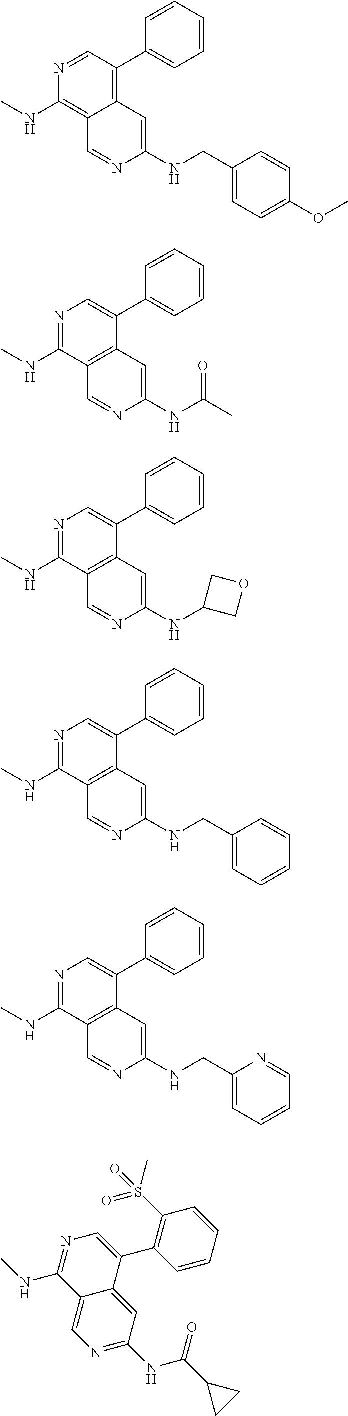

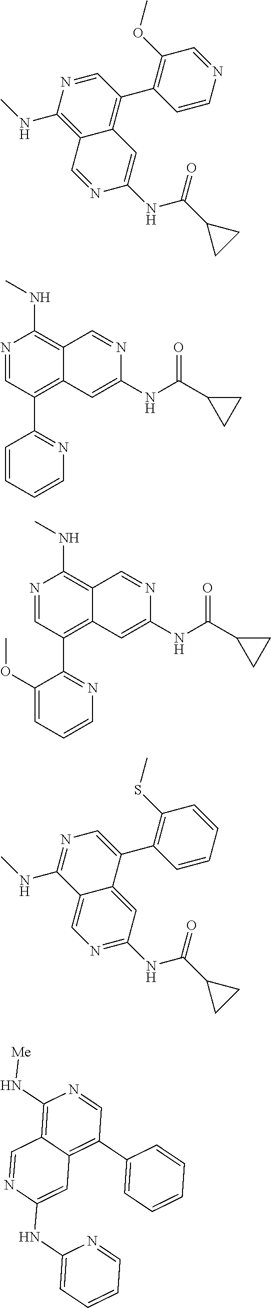

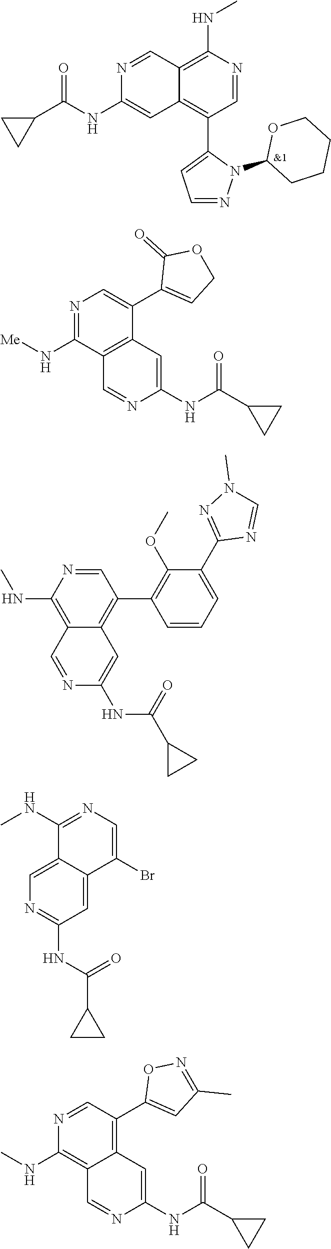

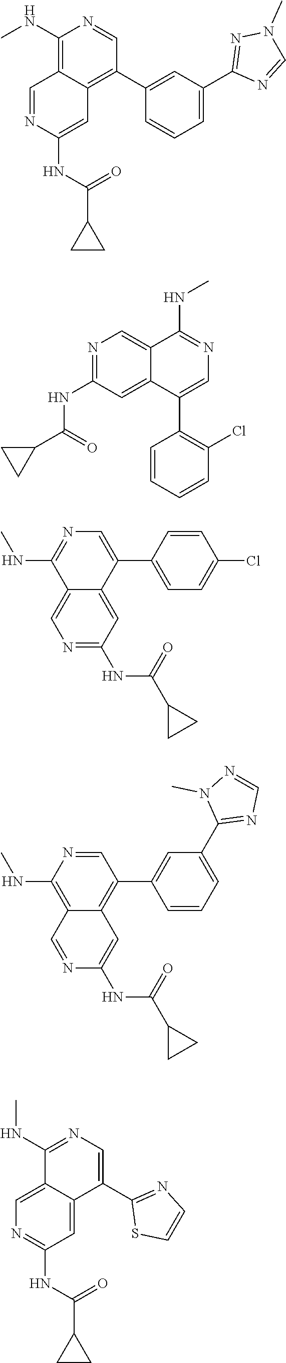

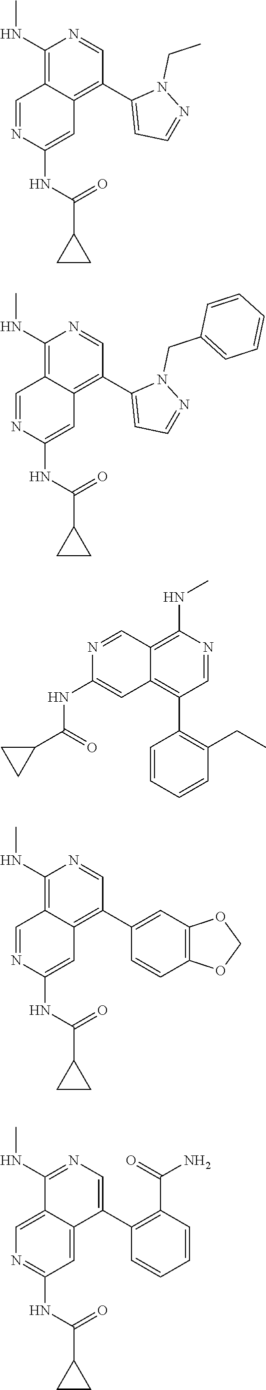 [SMILES: CCn1nccc1-c1cnc(NC)c2cnc(NC(=O)C3CC3)cc12 ; CNc1ncc(-c2ccnn2Cc2ccccc2)c2cc(NC(=O)C3CC3)ncc12 ; CCc1ccccc1-c1cnc(NC)c2cnc(NC(=O)C3CC3)cc12 ; CNc1ncc(-c2ccc3c(c2)OCO3)c2cc(NC(=O)C3CC3)ncc12 ; CNc1ncc(-c2ccccc2C(N)=O)c2cc(NC(=O)C3CC3)ncc12]

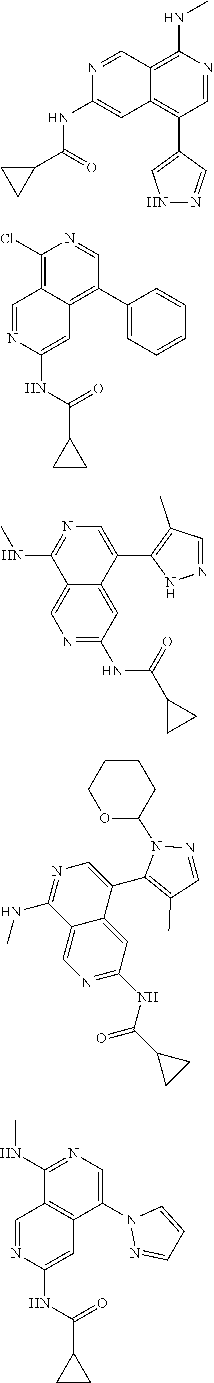

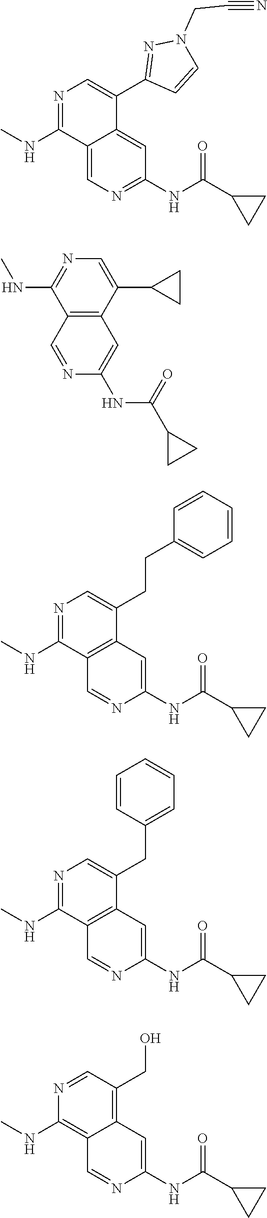

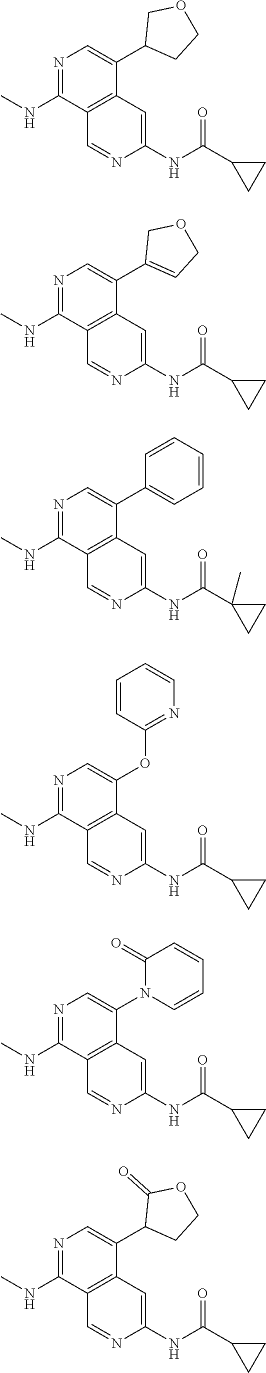 [SMILES: CNc1ncc(C2CCOC2)c2cc(NC(=O)C3CC3)ncc12 ; CNc1ncc(C2=CCOC2)c2cc(NC(=O)C3CC3)ncc12 ; CNc1ncc(-c2ccccc2)c2cc(NC(=O)C3(C)CC3)ncc12 ; CNc1ncc(Oc2ccccn2)c2cc(NC(=O)C3CC3)ncc12 ; CNc1ncc(-n2ccccc2=O)c2cc(NC(=O)C3CC3)ncc12 ; CNc1ncc(C2CCOC2=O)c2cc(NC(=O)C3CC3)ncc12]

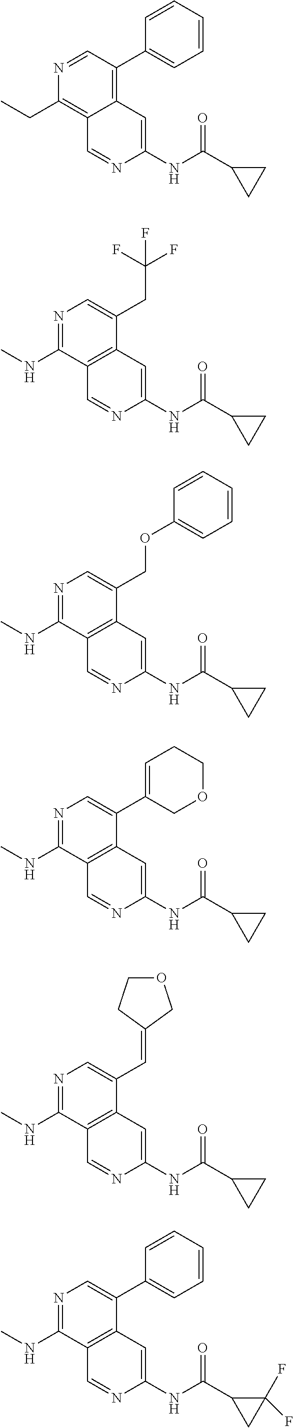

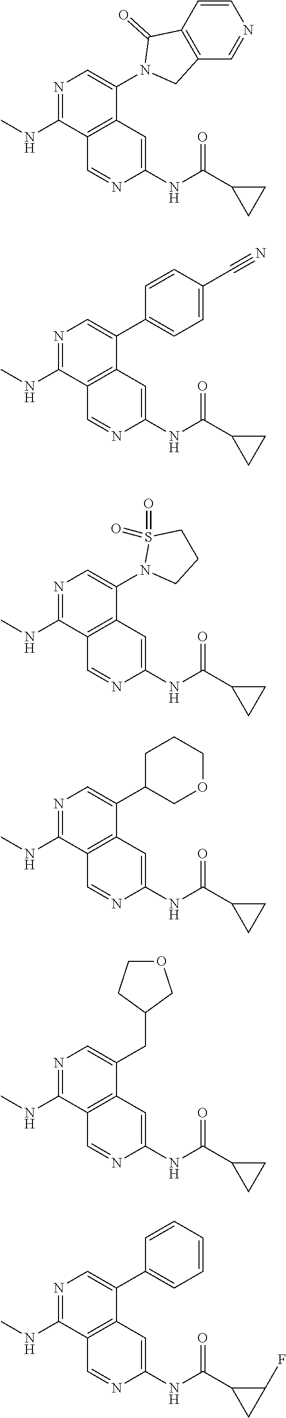

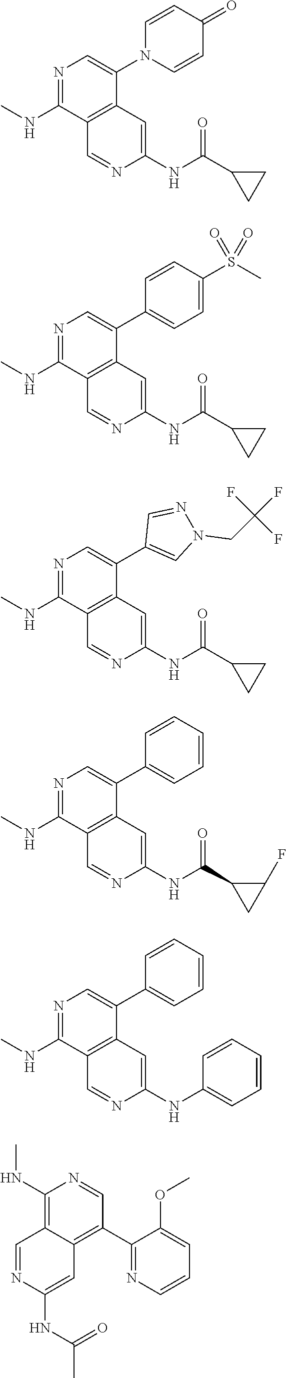

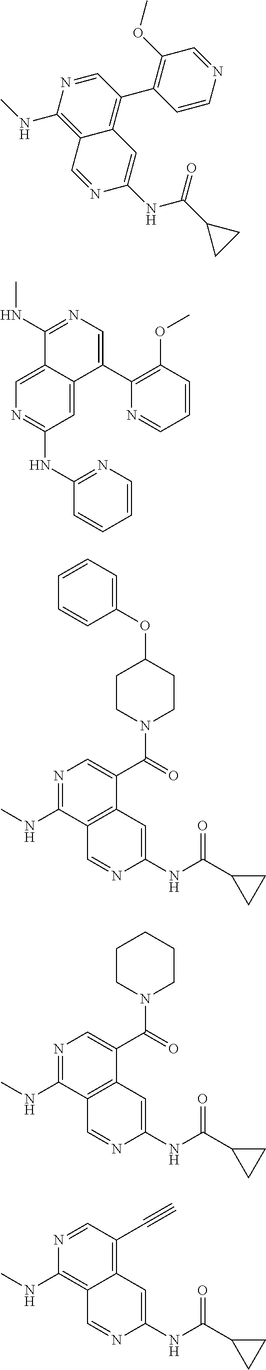

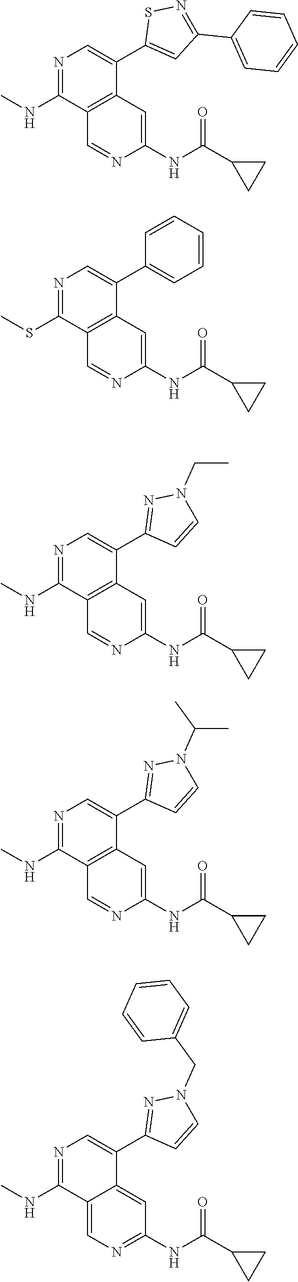 [SMILES: CNc1ncc(-c2cc(-c3ccccc3)ns2)c2cc(NC(=O)C3CC3)ncc12 ; CSc1ncc(-c2ccccc2)c2cc(NC(=O)C3CC3)ncc12 ; CCn1ccc(-c2cnc(NC)c3cnc(NC(=O)C4CC4)cc23)n1 ; CNc1ncc(-c2ccn(C(C)C)n2)c2cc(NC(=O)C3CC3)ncc12 ; CNc1ncc(-c2ccn(Cc3ccccc3)n2)c2cc(NC(=O)C3CC3)ncc12]

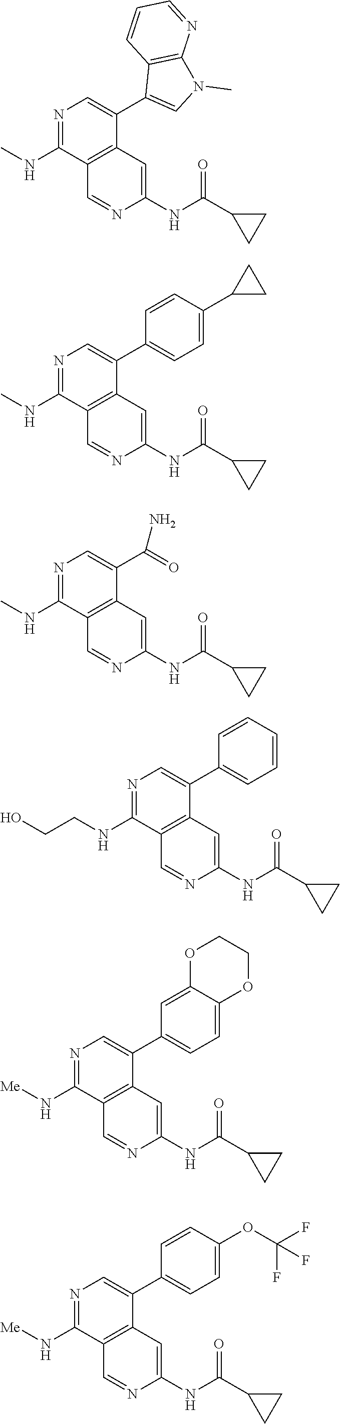

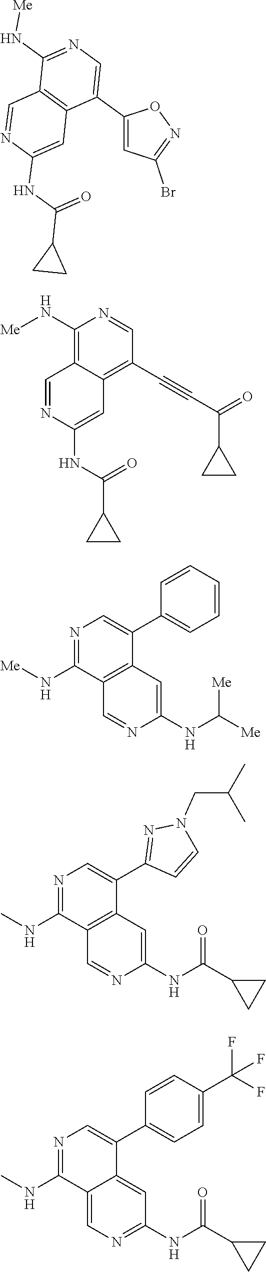

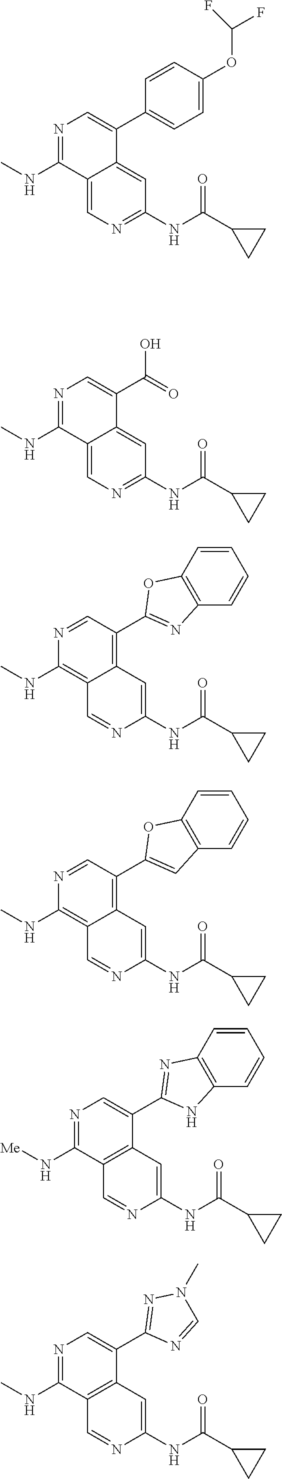

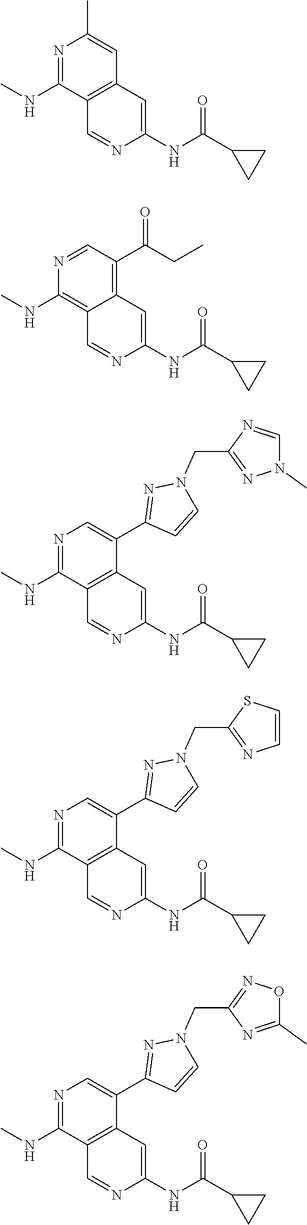

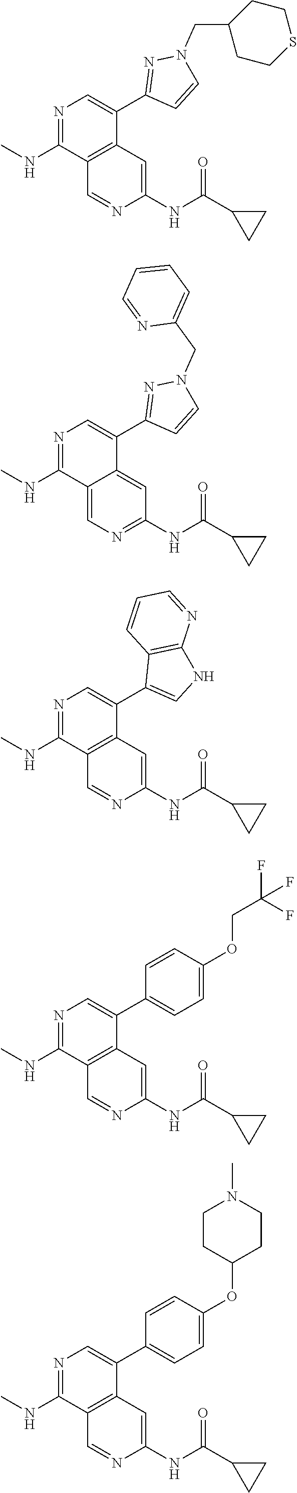

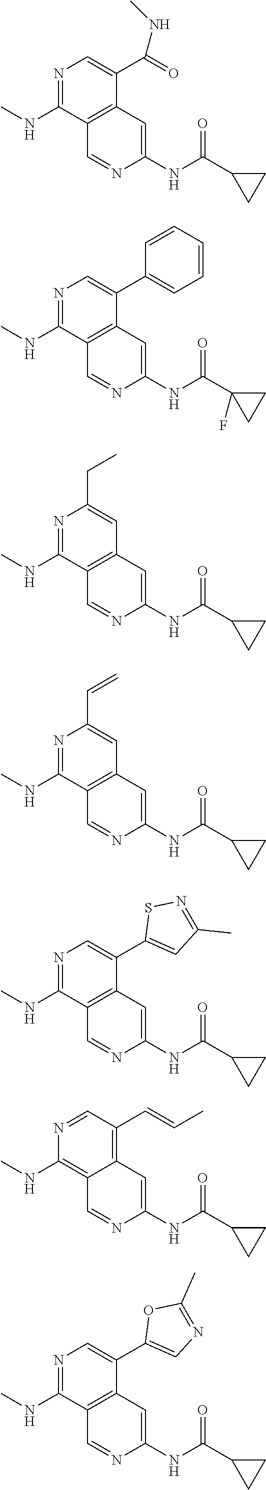

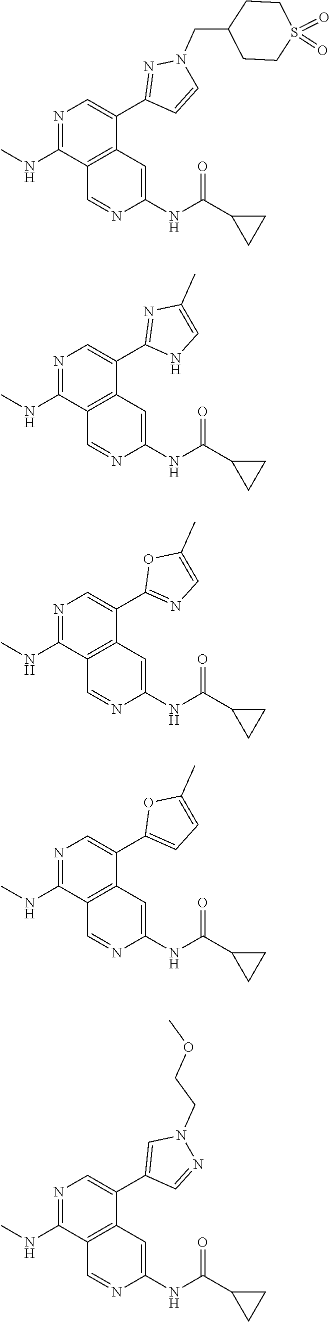 [SMILES: CNc1ncc(-c2ccn(CC3CCS(=O)(=O)CC3)n2)c2cc(NC(=O)C3CC3)ncc12 ; CNc1ncc(-c2nc(C)c[nH]2)c2cc(NC(=O)C3CC3)ncc12 ; CNc1ncc(-c2ncc(C)o2)c2cc(NC(=O)C3CC3)ncc12 ; CNc1ncc(-c2ccc(C)o2)c2cc(NC(=O)C3CC3)ncc12 ; CNc1ncc(-c2cnn(CCOC)c2)c2cc(NC(=O)C3CC3)ncc12]

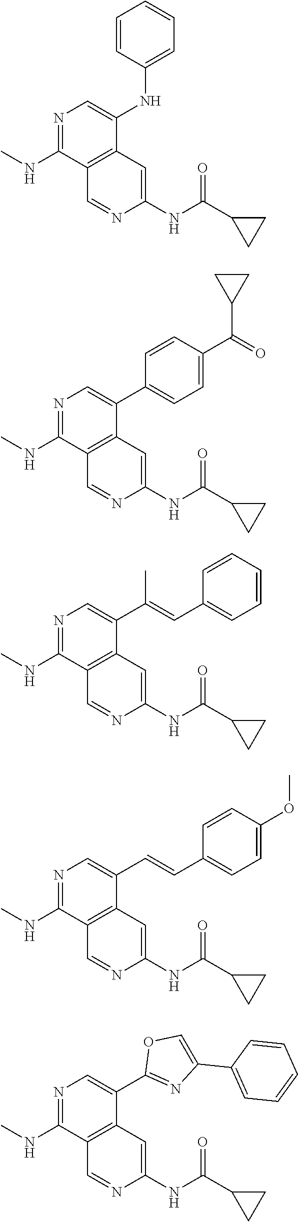

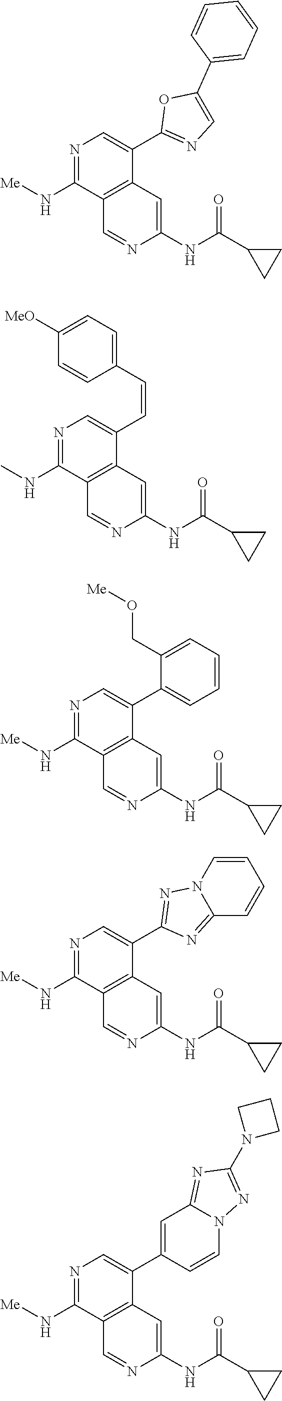

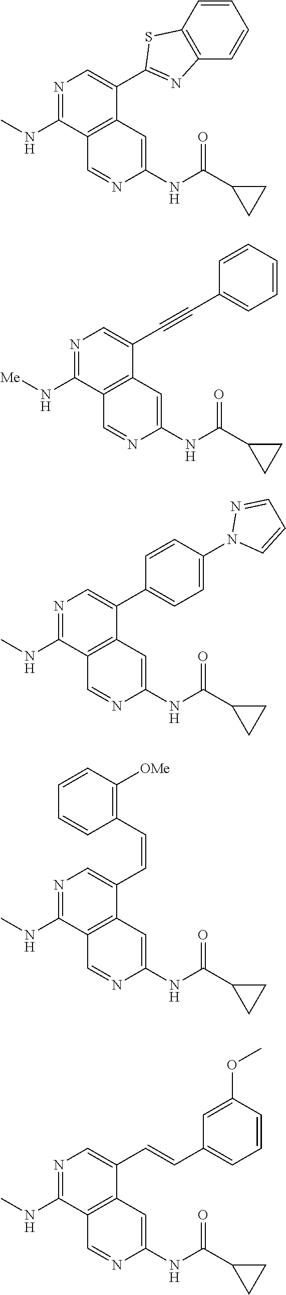

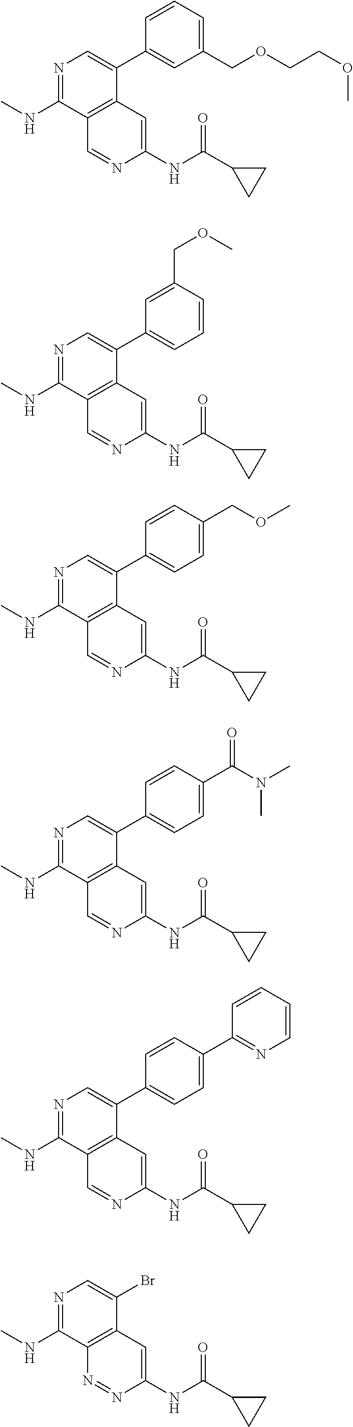

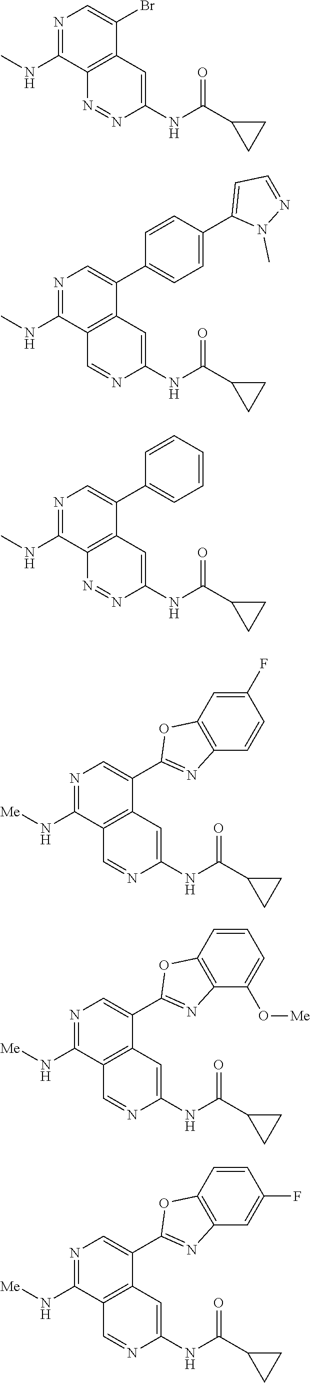

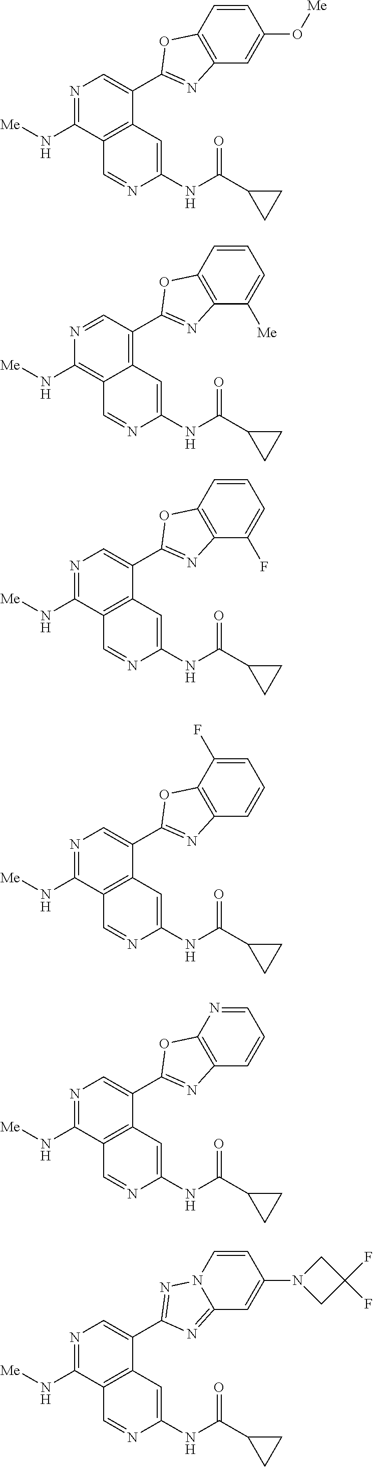

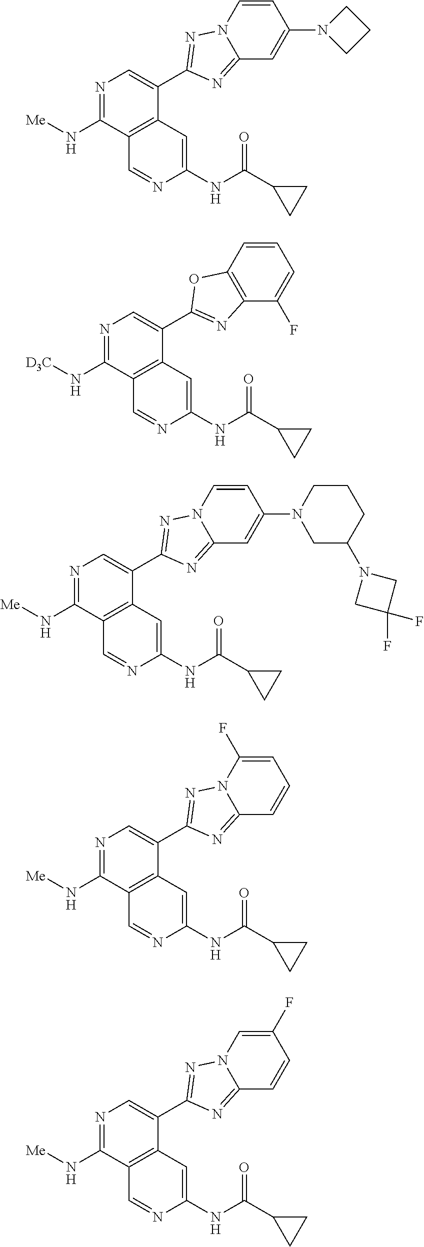

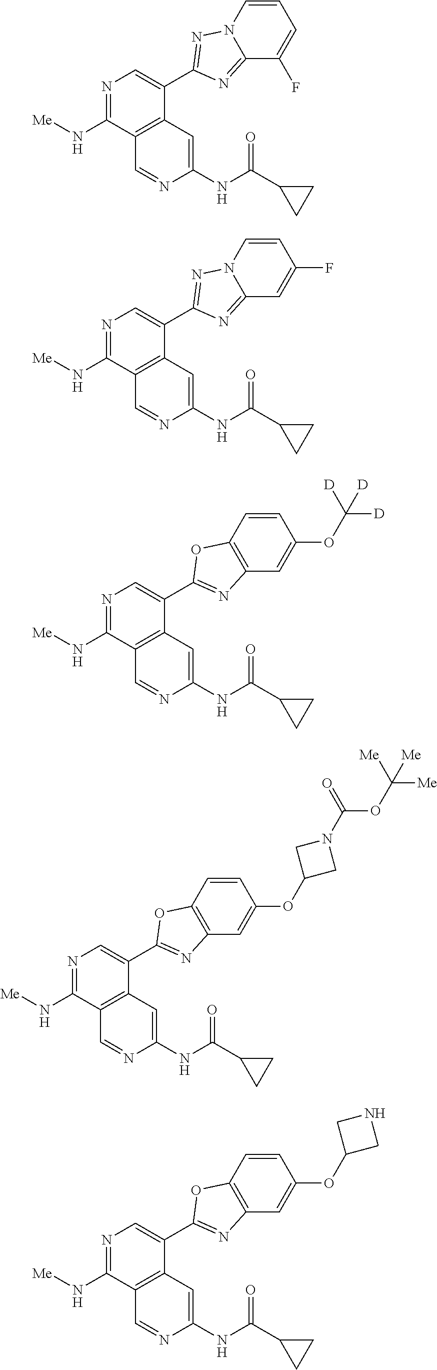

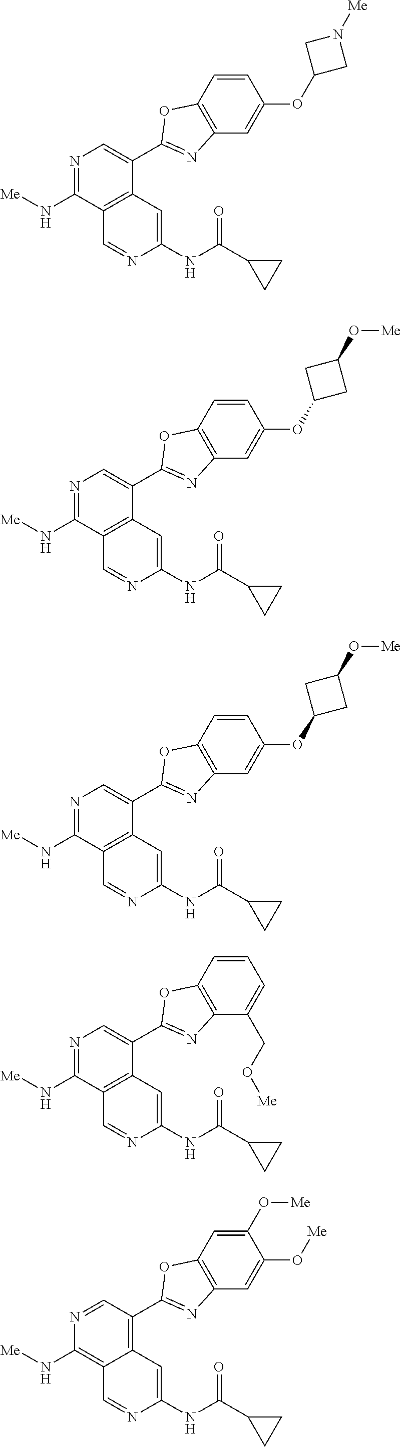

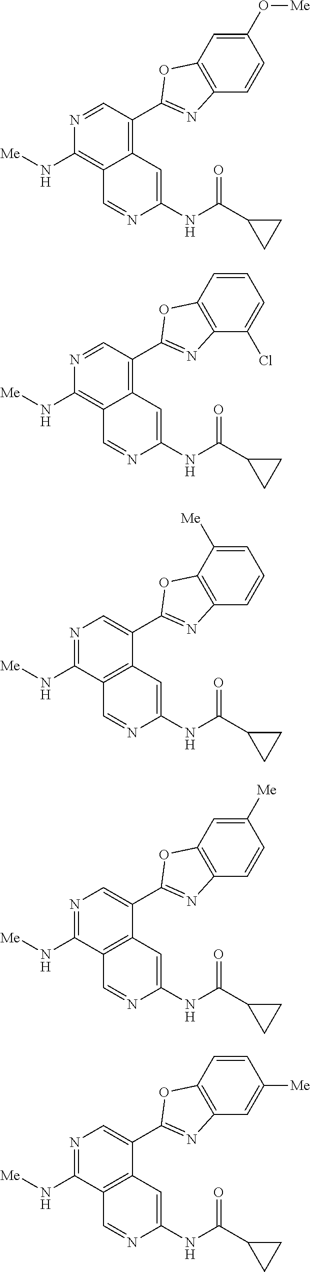

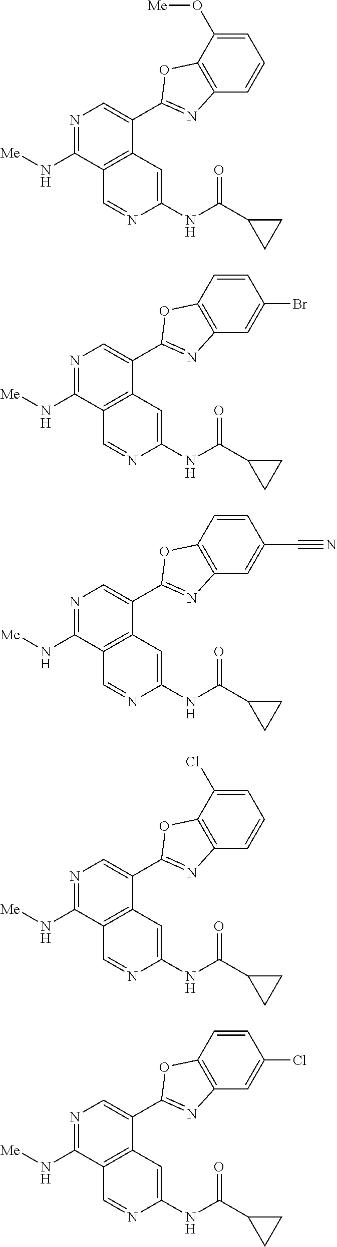

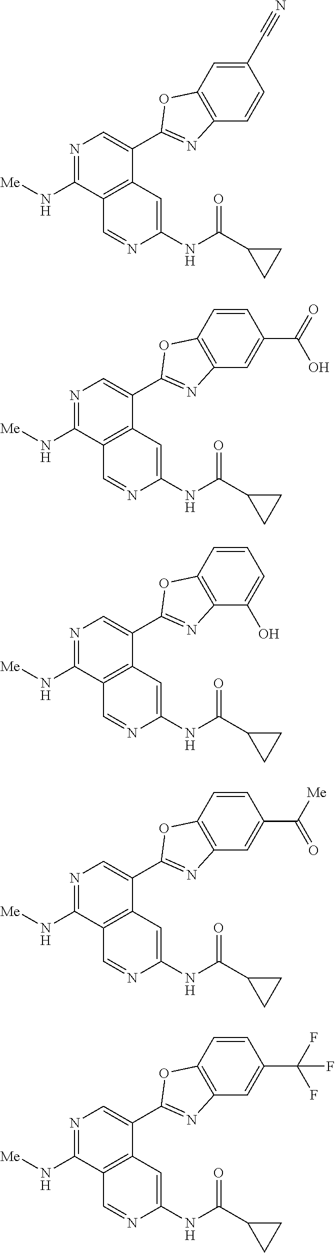

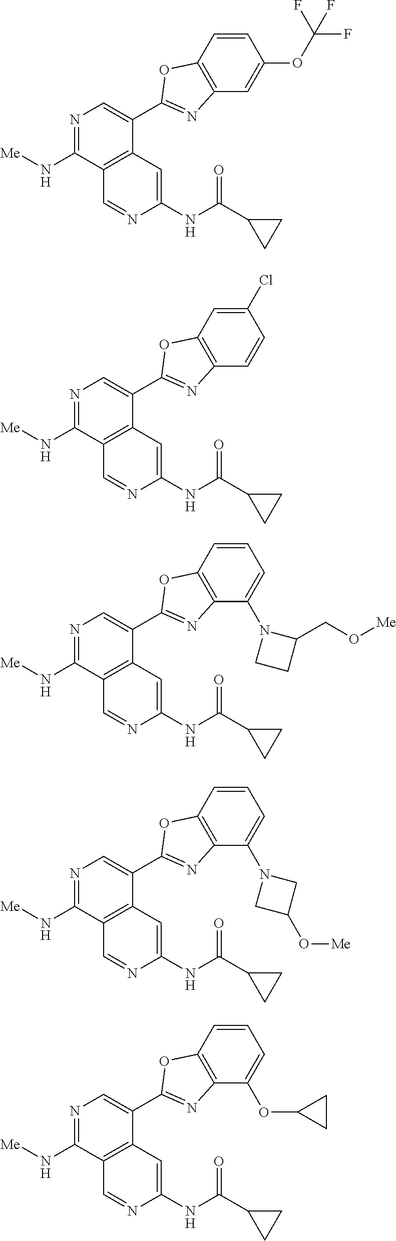

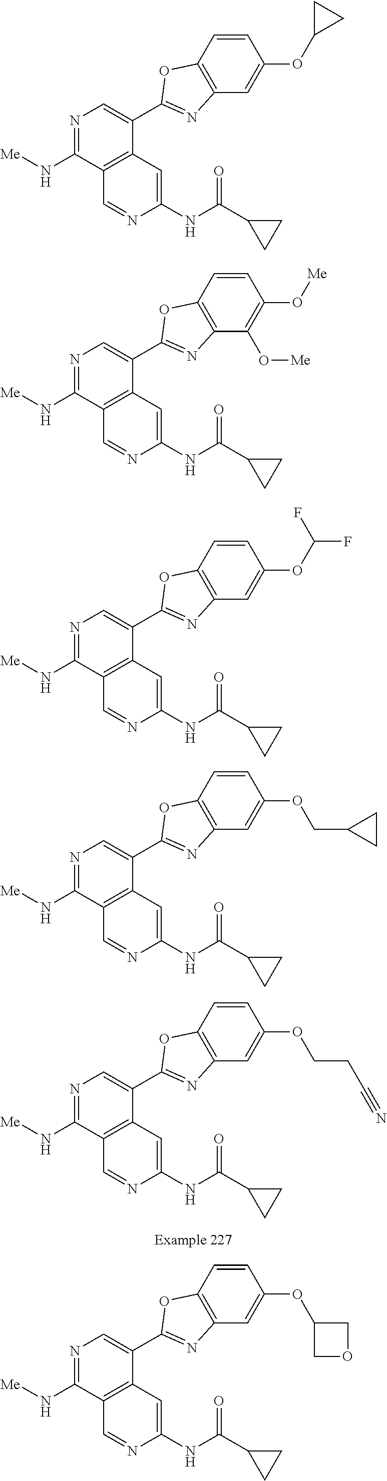

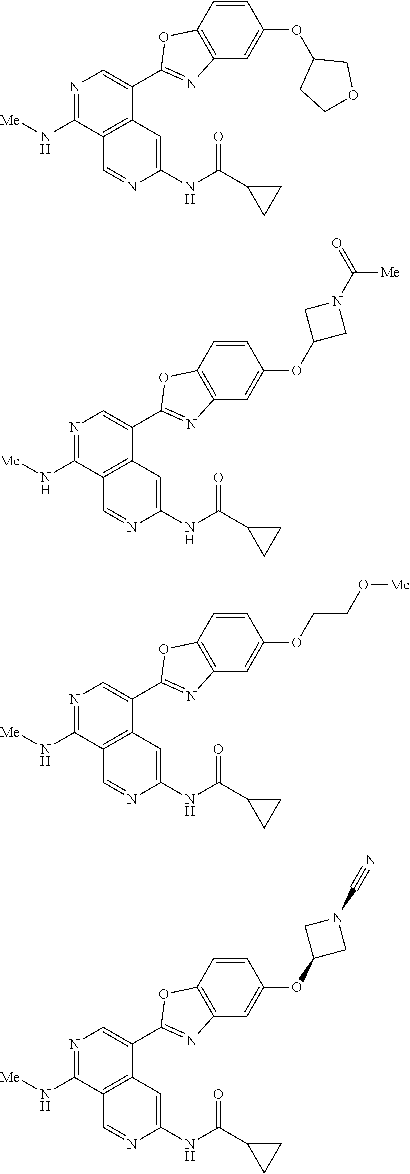

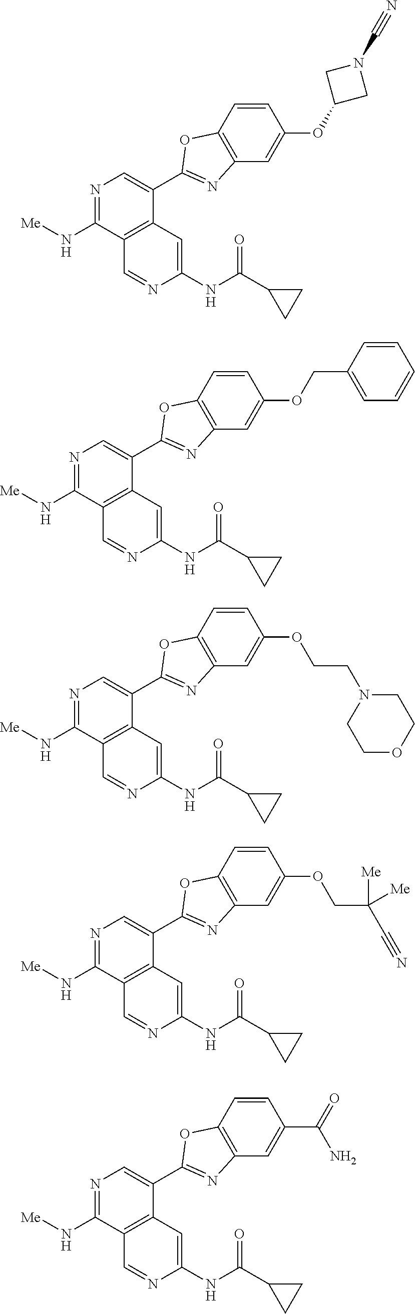

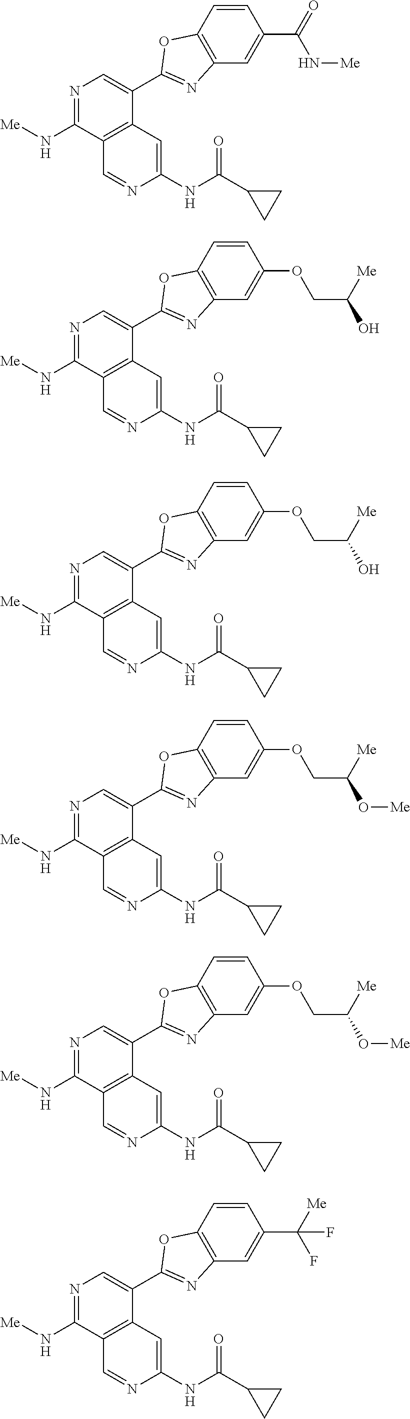

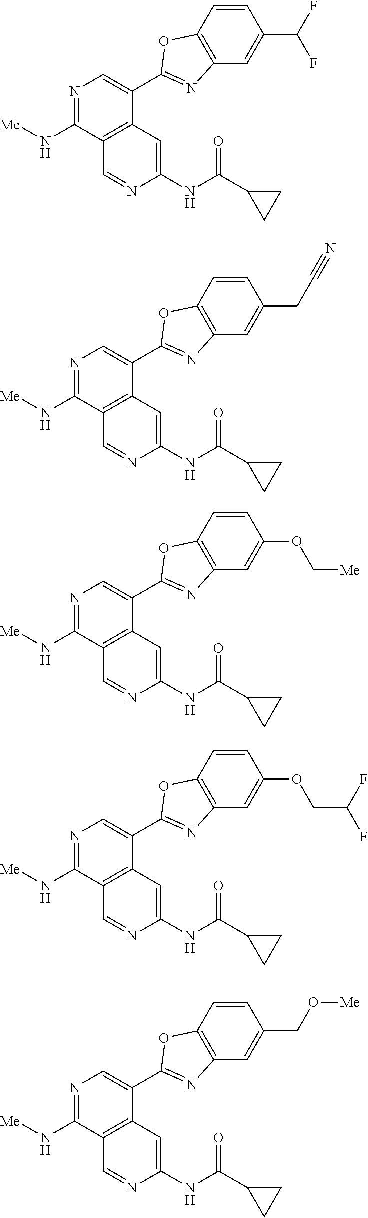

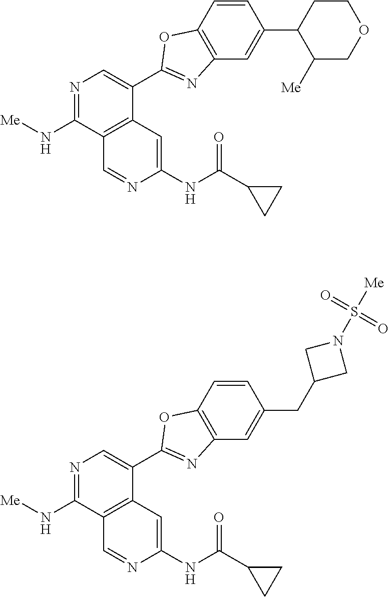 [SMILES: CNc1ncc(-c2nc3cc(C4CCOCC4C)ccc3o2)c2cc(NC(=O)C3CC3)ncc12 ; CNc1ncc(-c2nc3cc(CC4CN(S(C)(=O)=O)C4)ccc3o2)c2cc(NC(=O)C3CC3)ncc12]

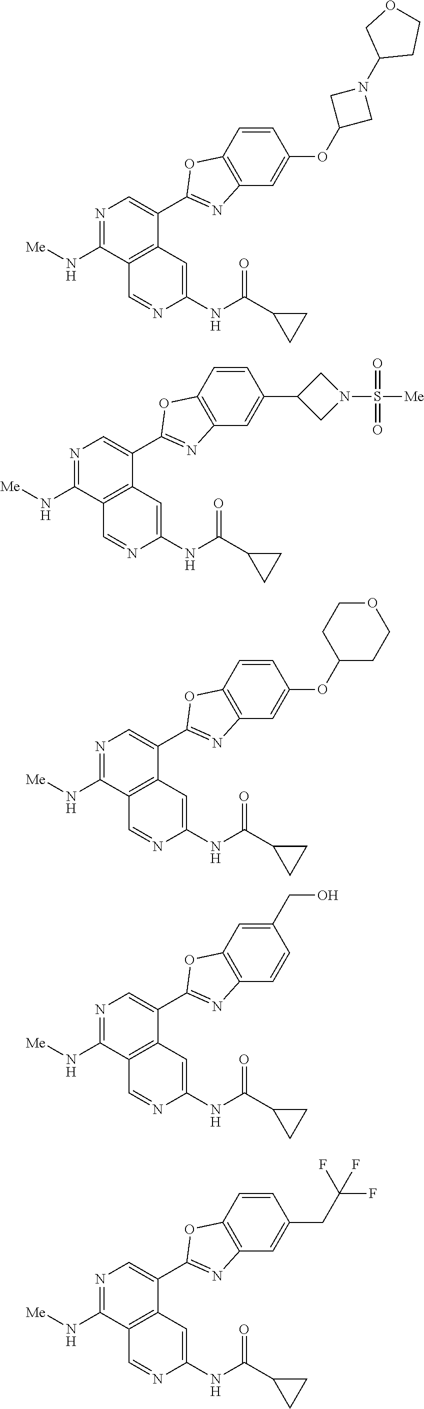

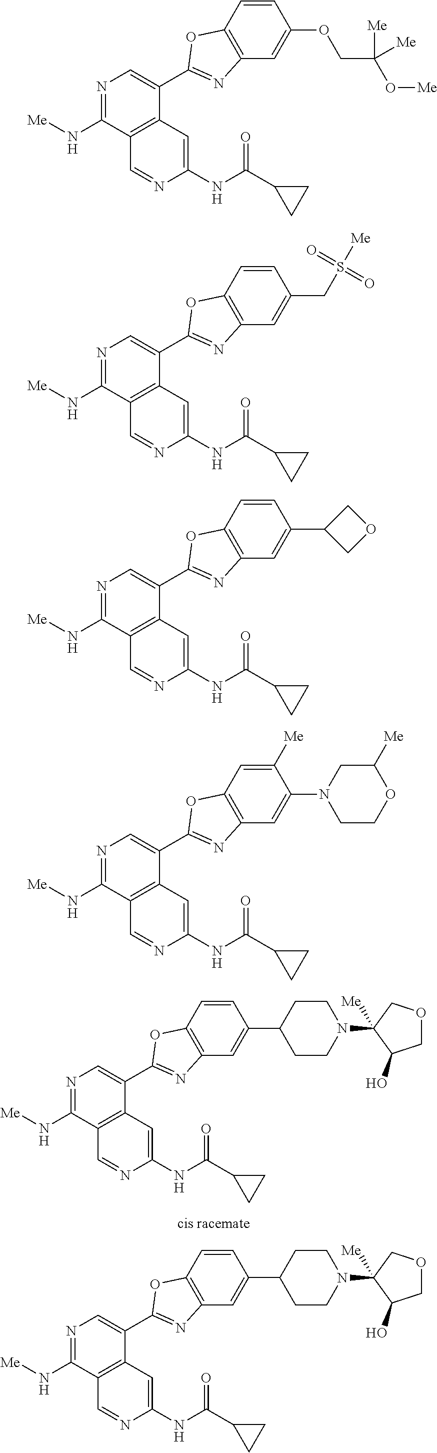

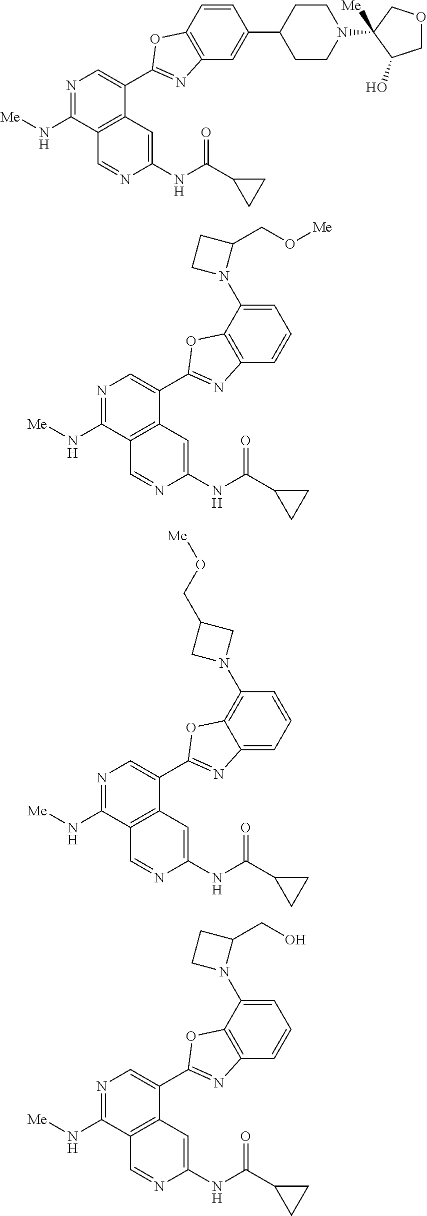

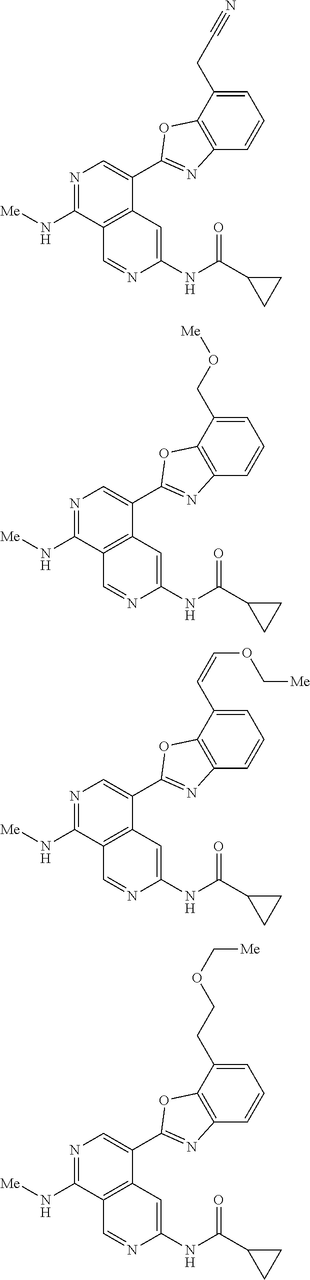

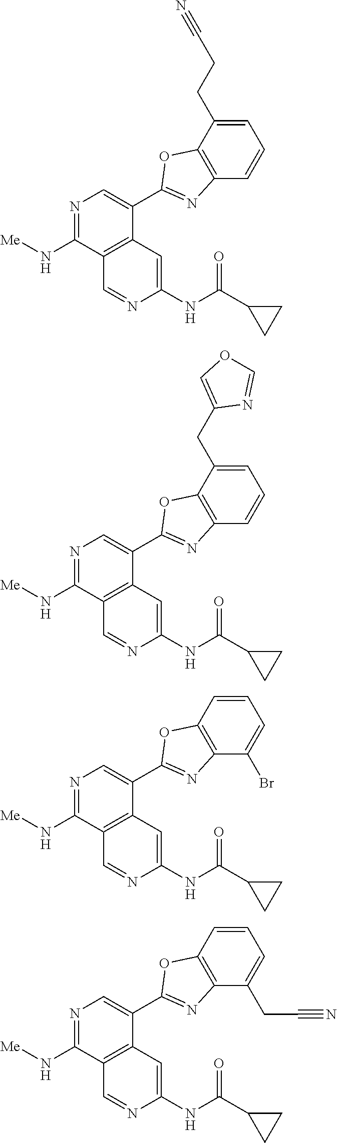

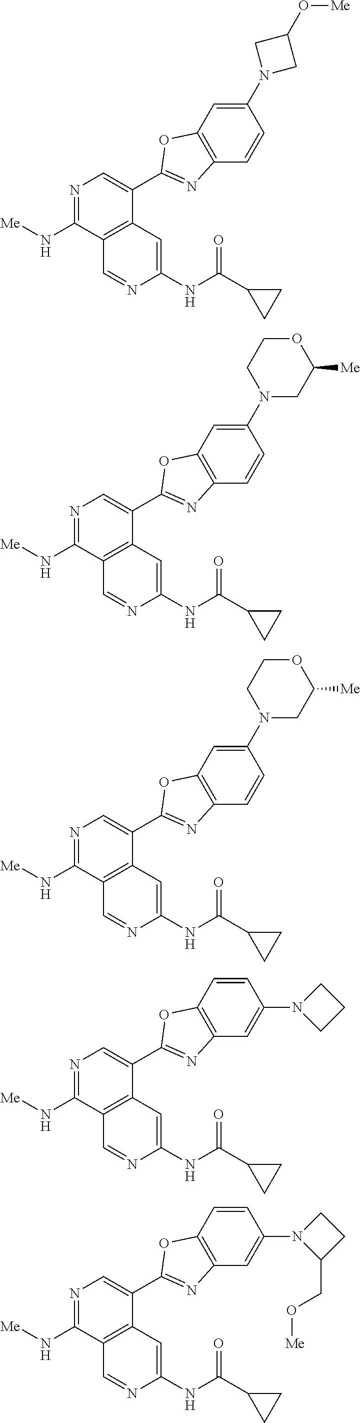

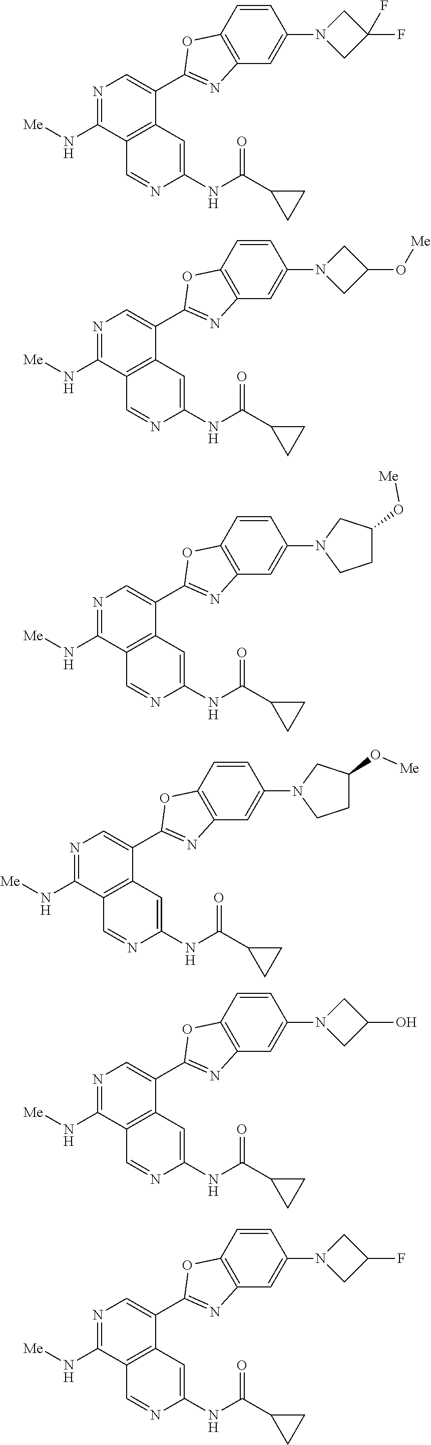

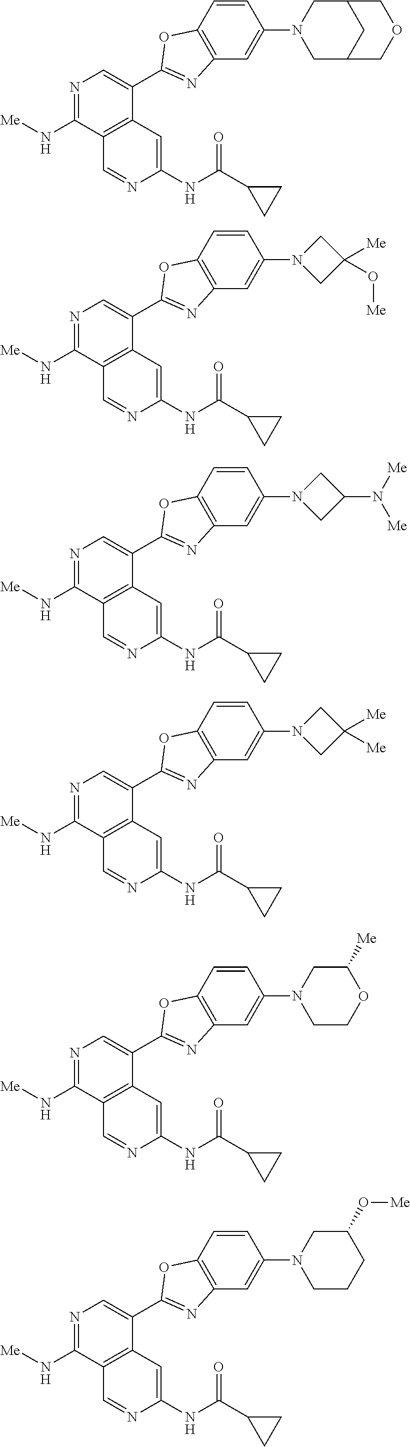

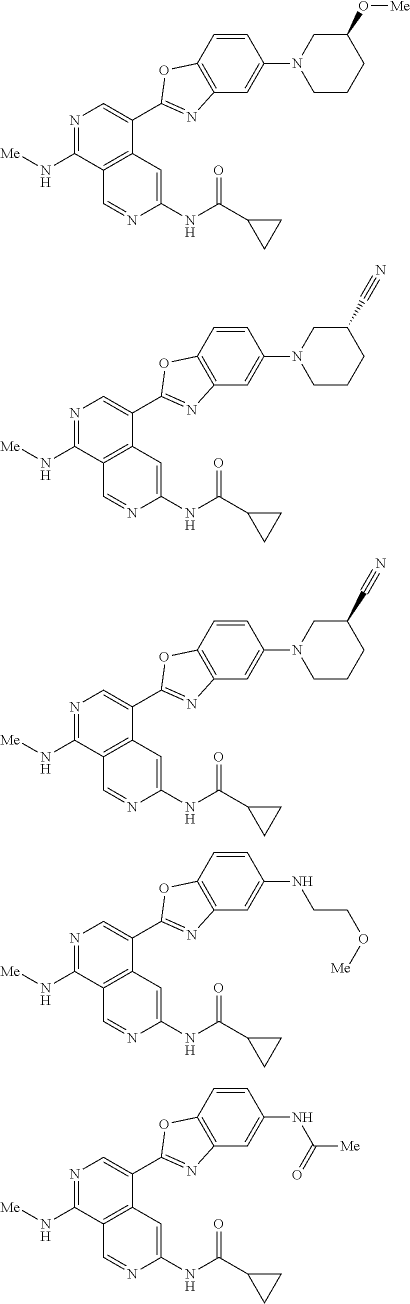

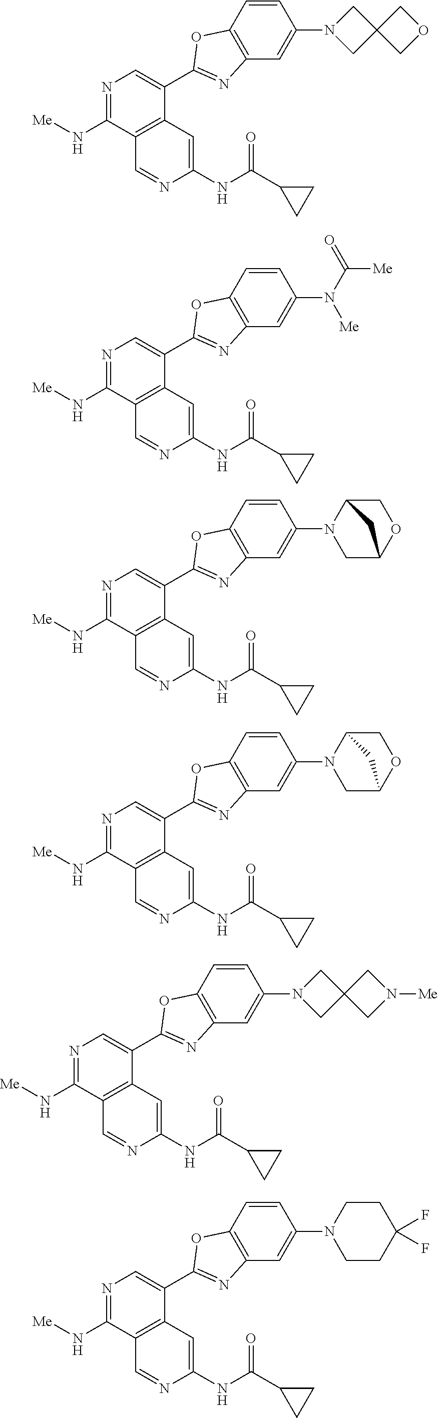

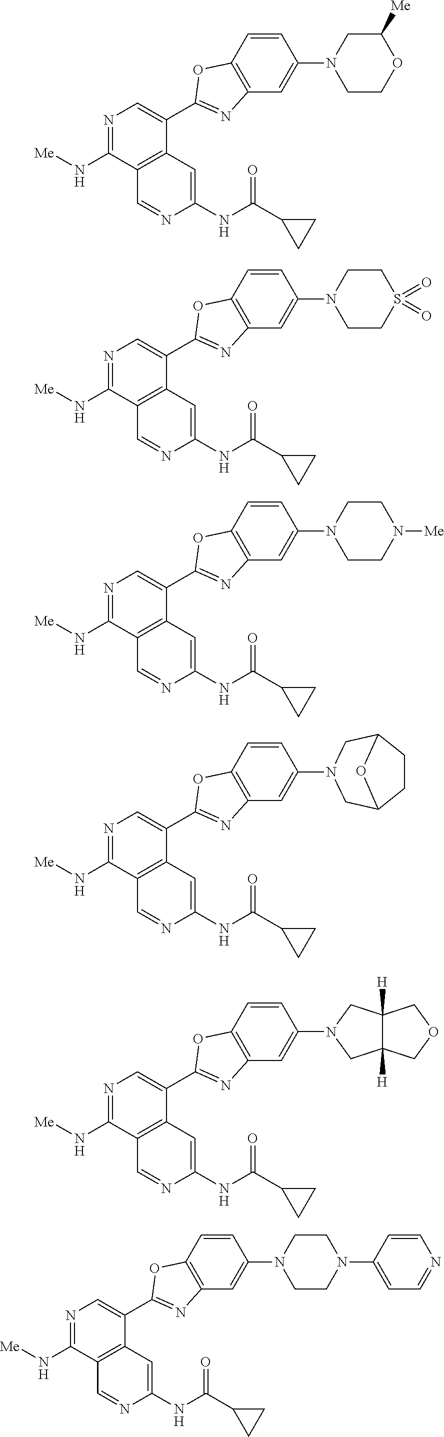

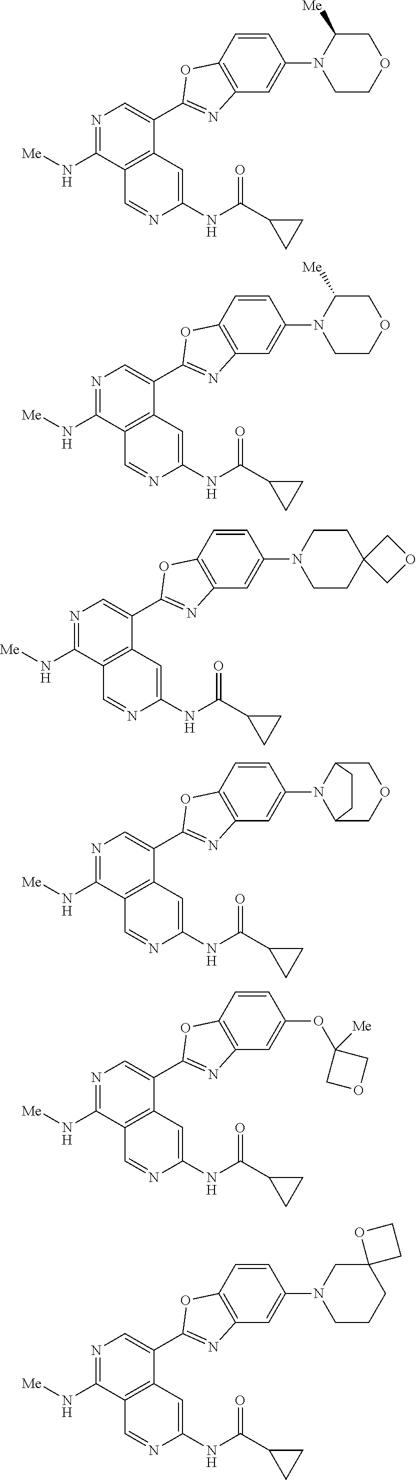

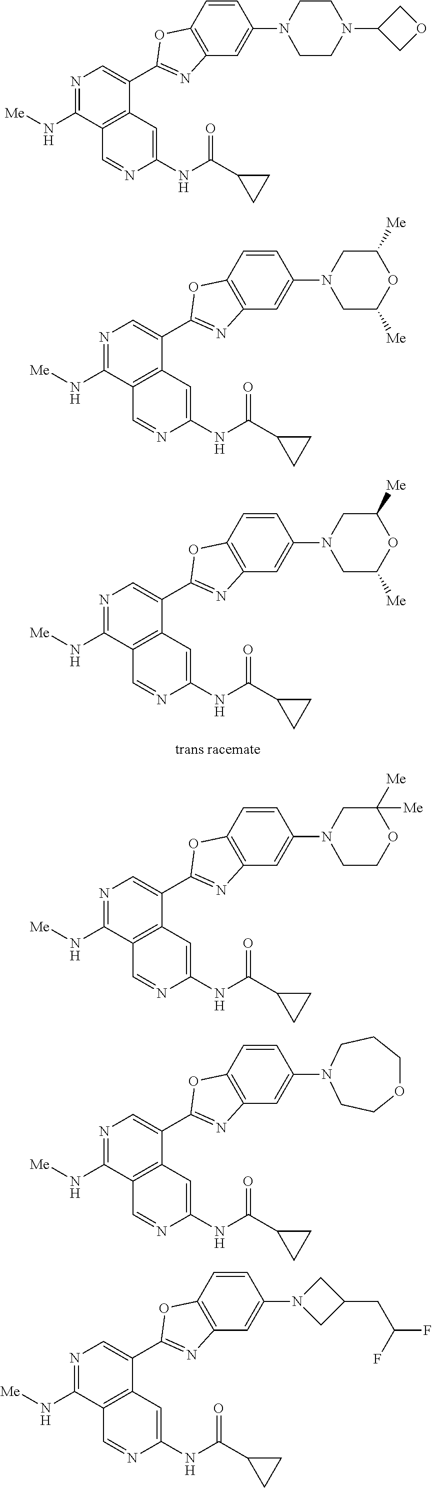

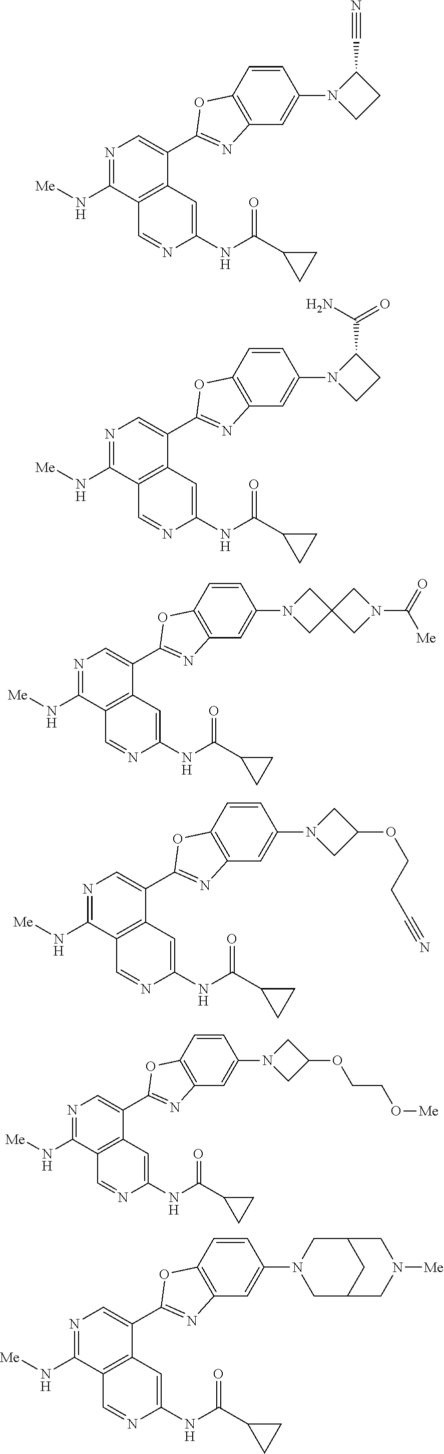

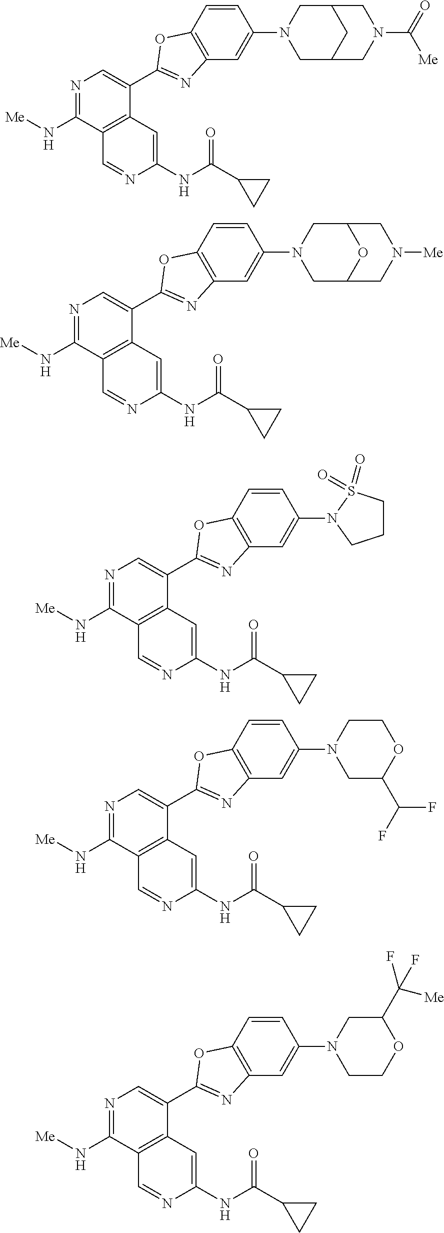

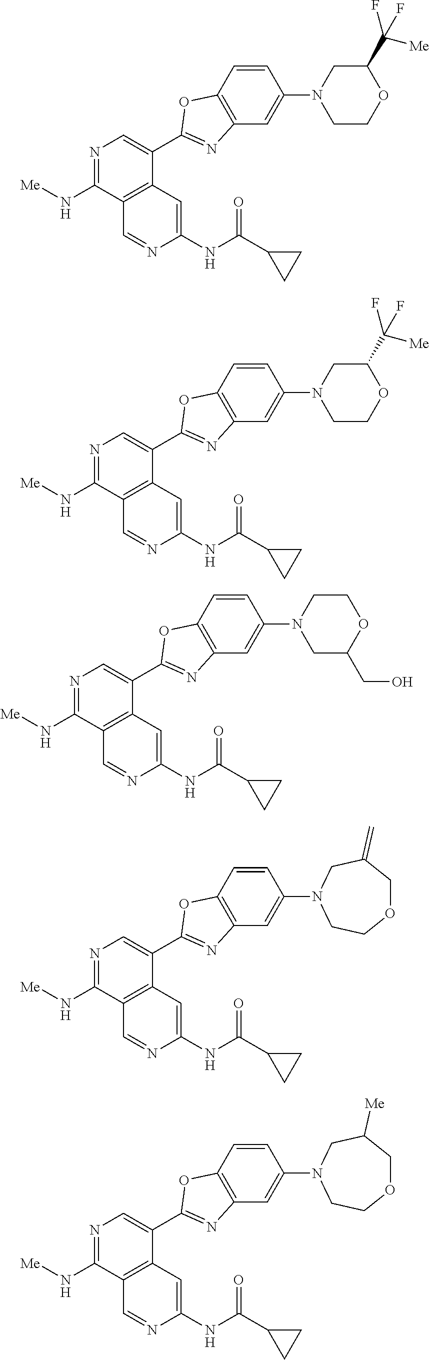

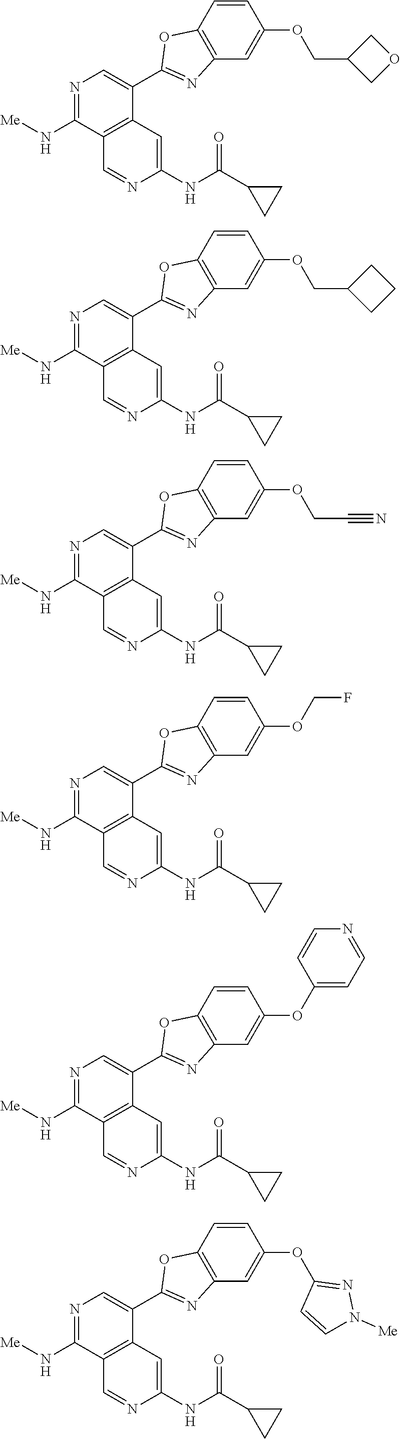

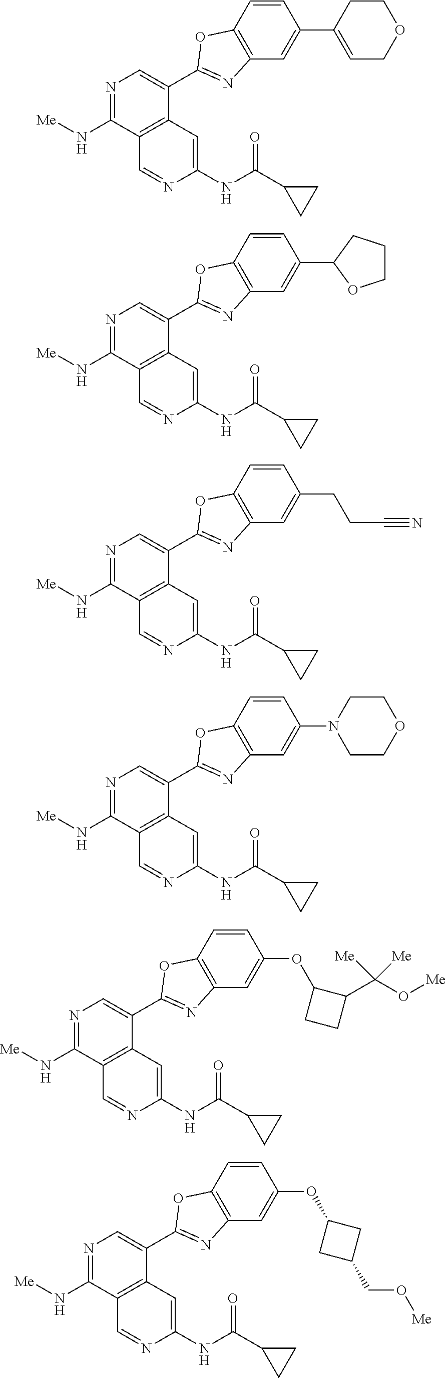

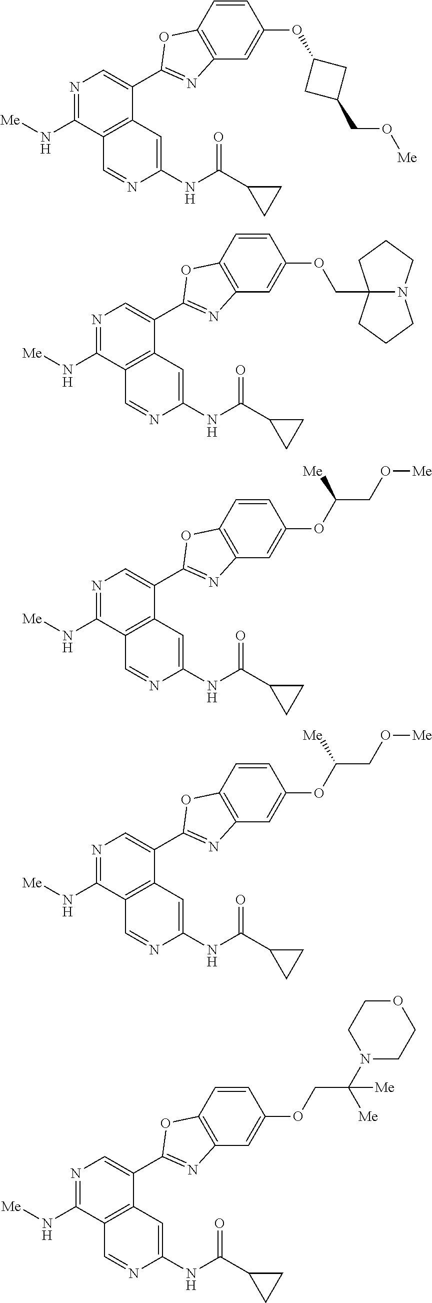

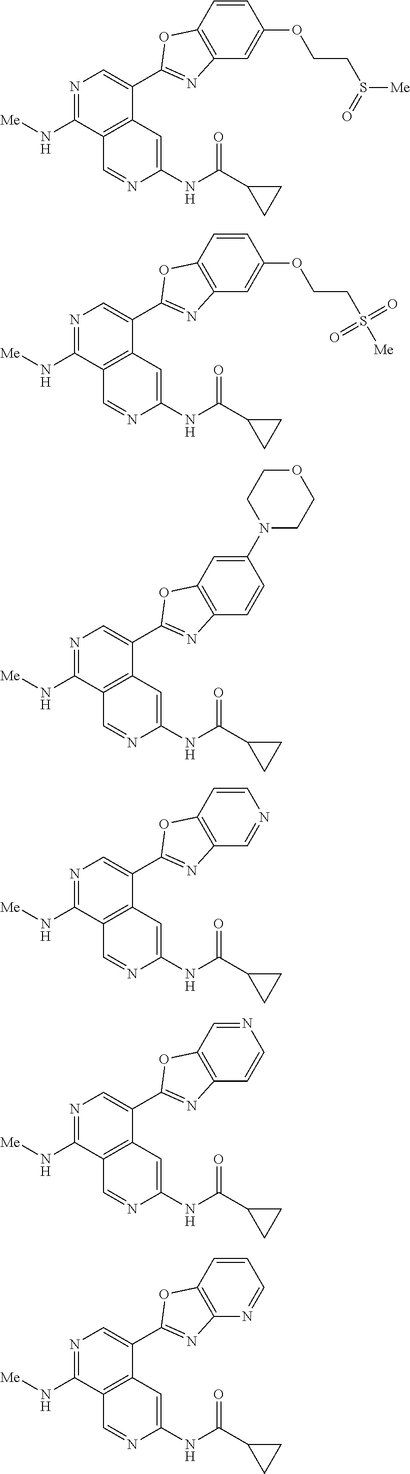

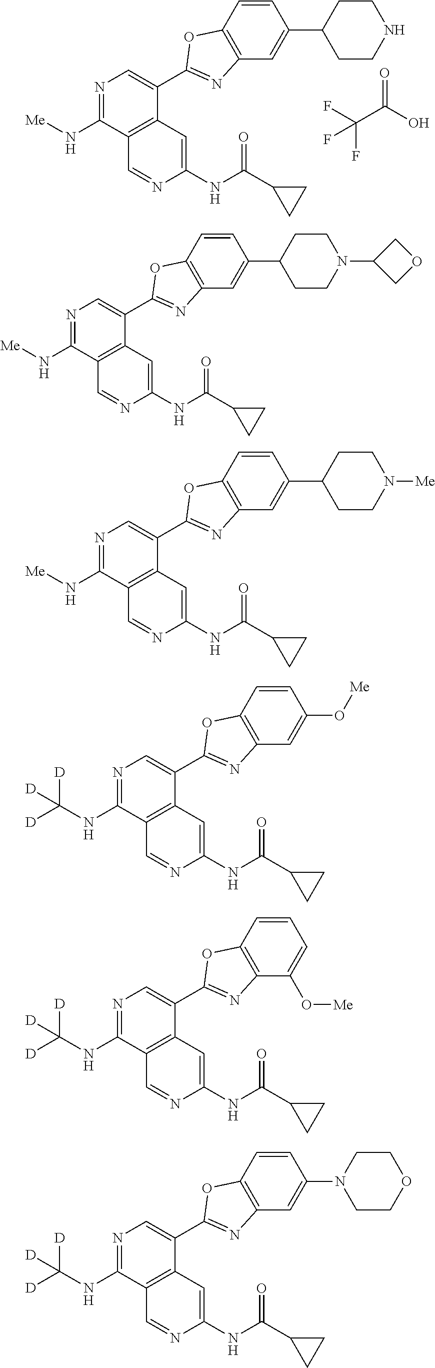

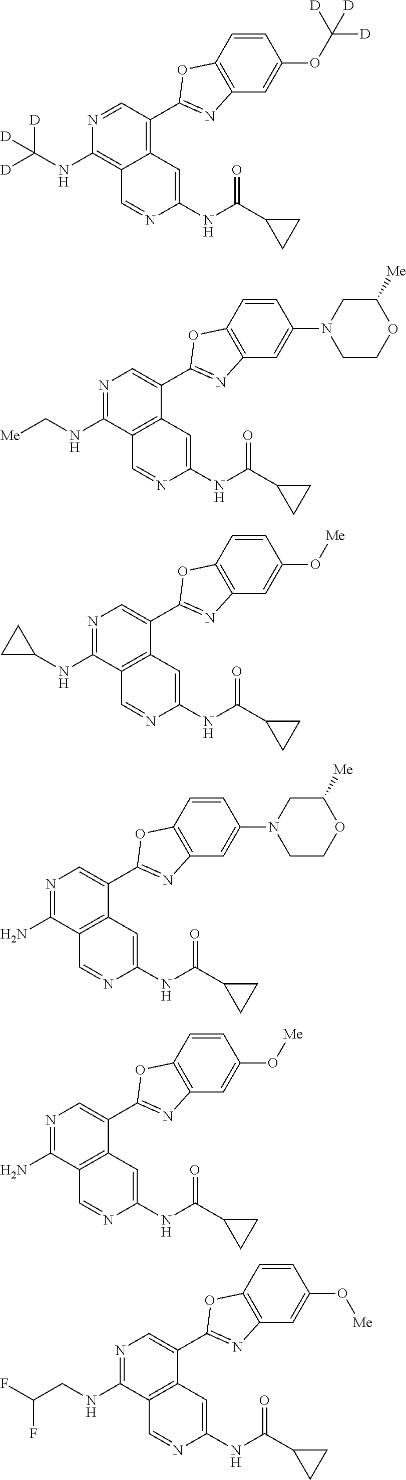

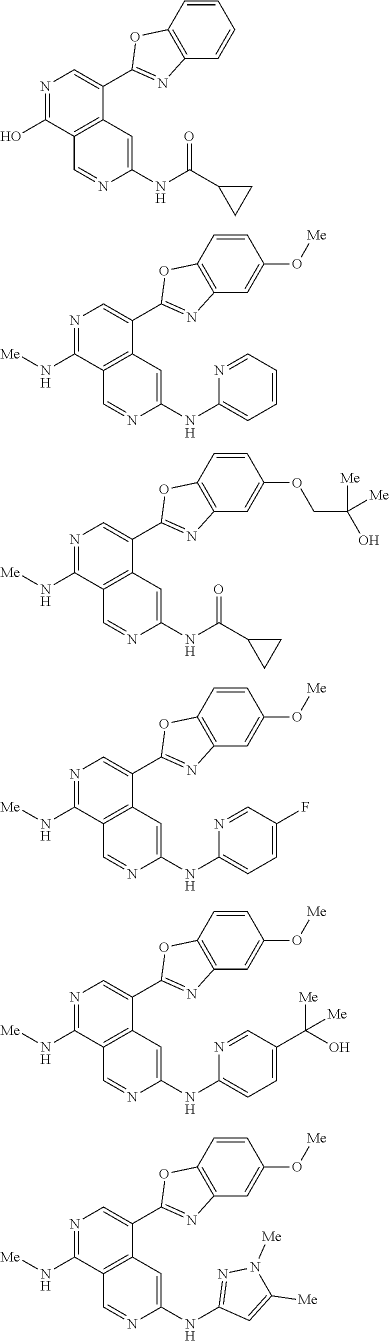

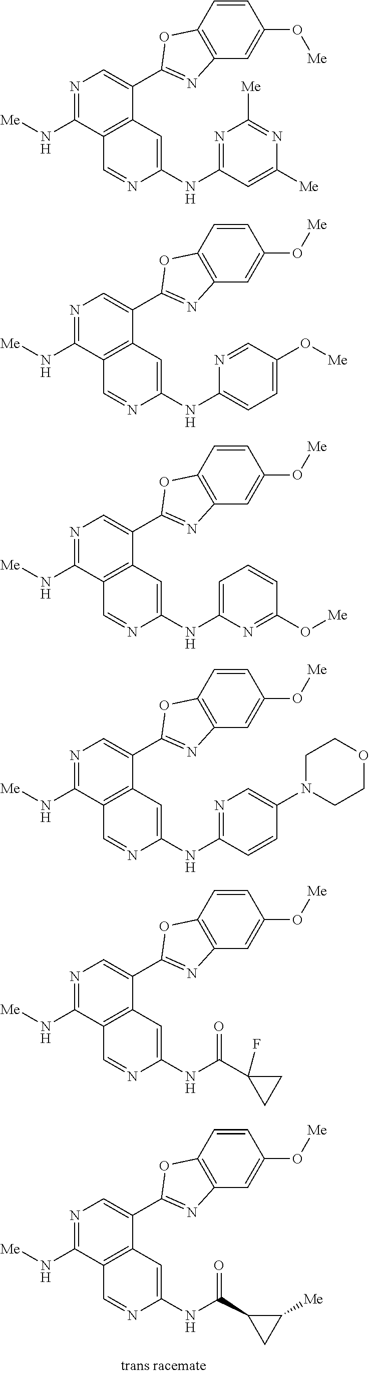

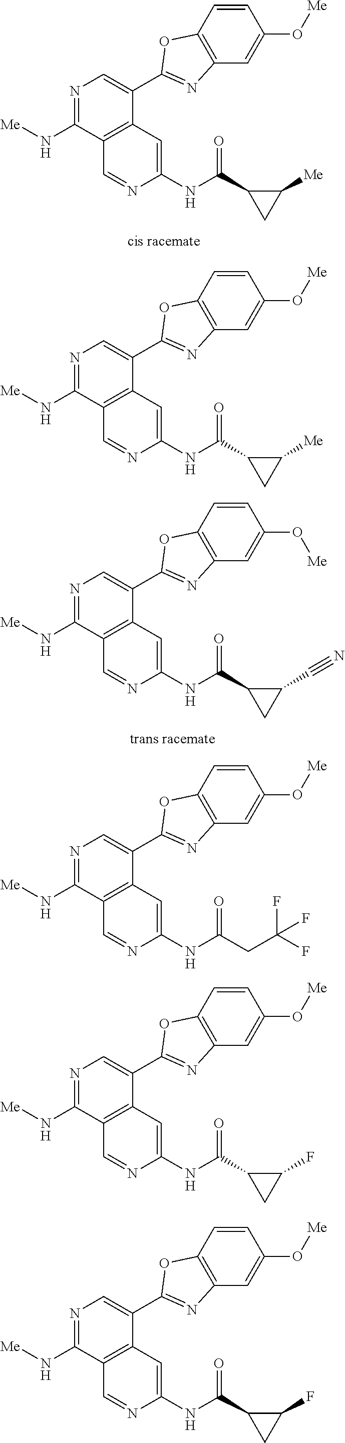

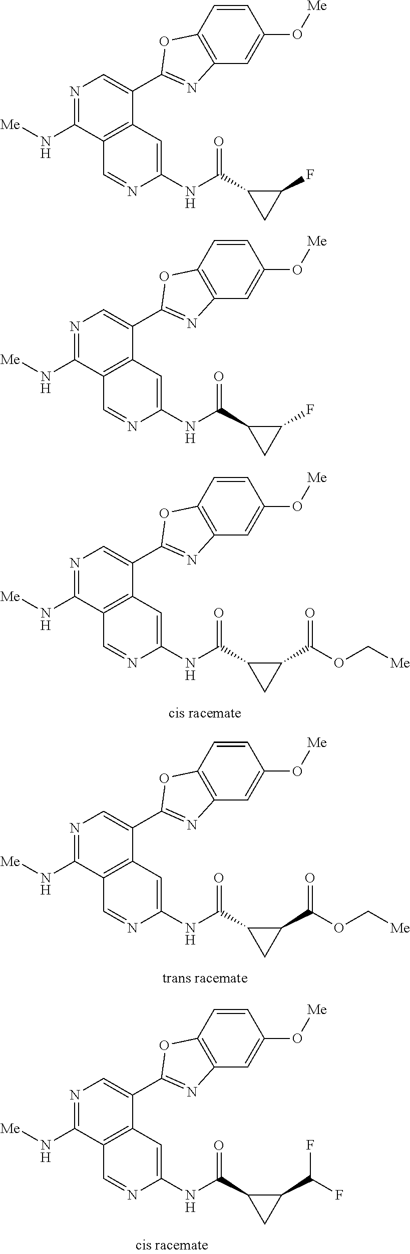

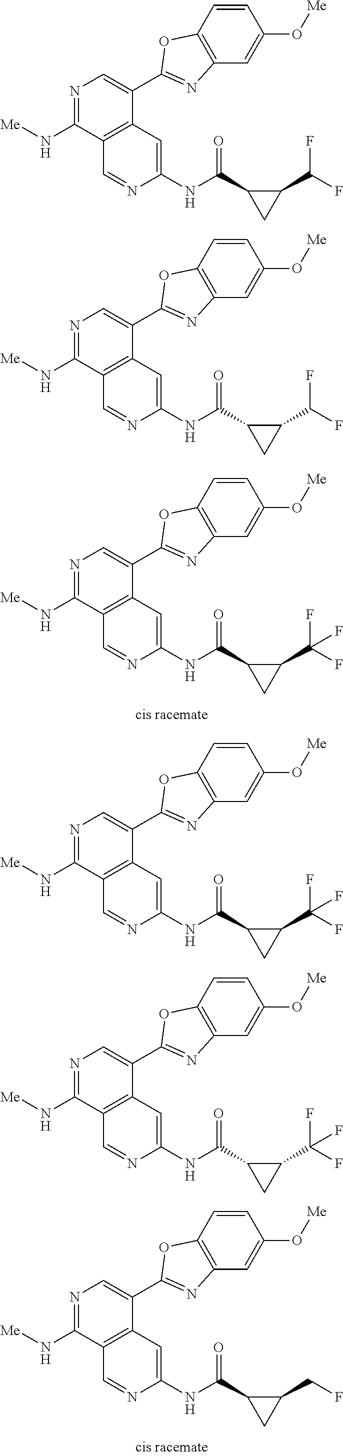

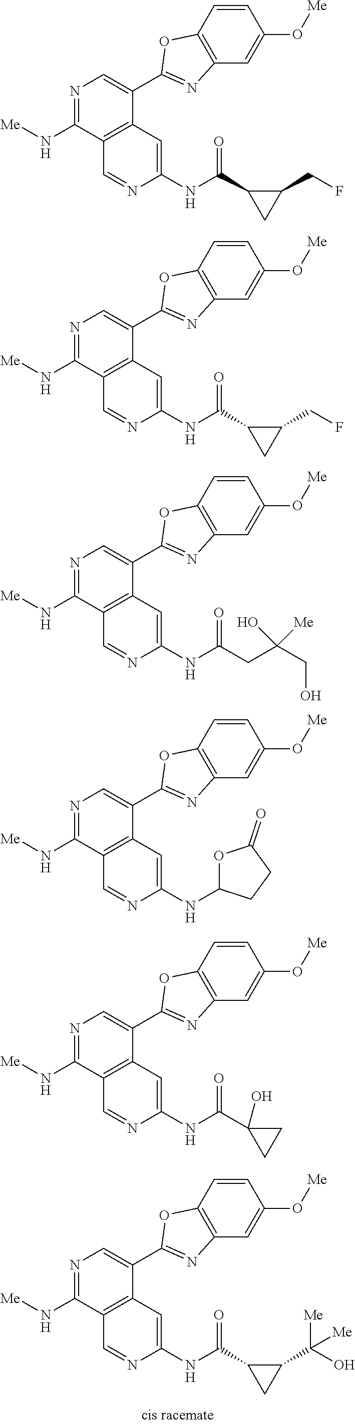

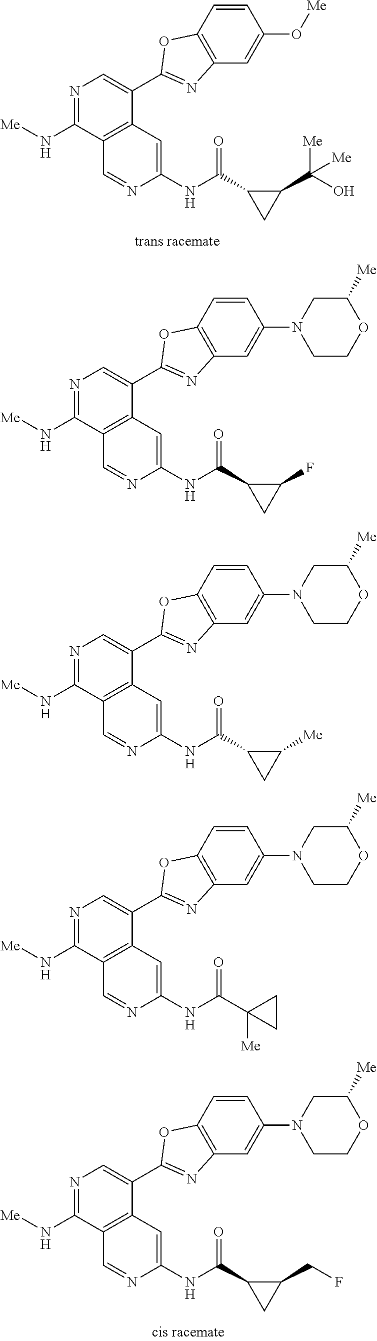 [SMILES: CNc1ncc(-c2nc3cc(OC)ccc3o2)c2cc(NC(=O)[C@H]3C[C@@H]3C(C)(C)O)ncc12 ; CNc1ncc(-c2nc3cc(N4CCO[C@@H](C)C4)ccc3o2)c2cc(NC(=O)[C@@H]3C[C@@H]3F)ncc12 ; CNc1ncc(-c2nc3cc(N4CCO[C@@H](C)C4)ccc3o2)c2cc(NC(=O)[C@H]3C[C@H]3C)ncc12 ; CNc1ncc(-c2nc3cc(N4CCO[C@@H](C)C4)ccc3o2)c2cc(NC(=O)C3(C)CC3)ncc12 ; CNc1ncc(-c2nc3cc(N4CCO[C@@H](C)C4)ccc3o2)c2cc(NC(=O)[C@@H]3C[C@@H]3CF)ncc12]

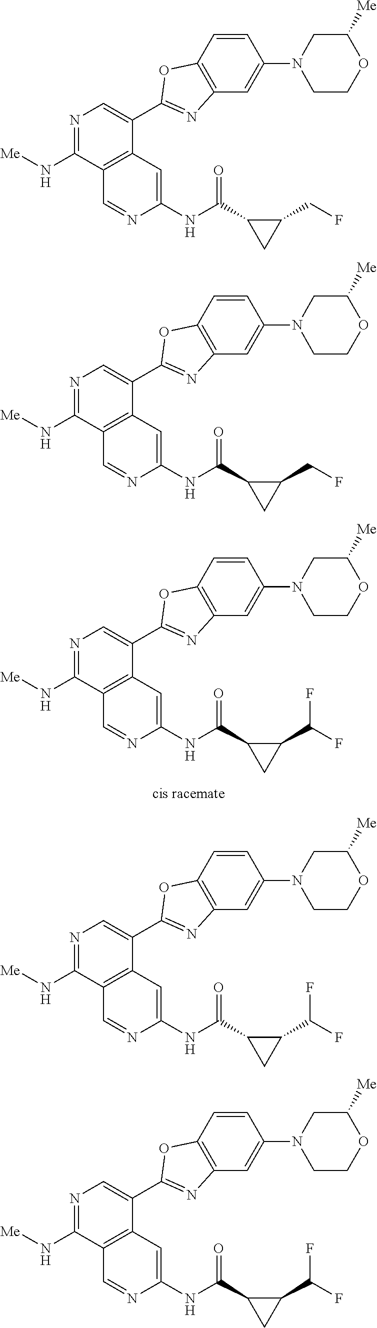 [SMILES: CNc1ncc(-c2nc3cc(N4CCO[C@@H](C)C4)ccc3o2)c2cc(NC(=O)[C@H]3C[C@H]3CF)ncc12 ; CNc1ncc(-c2nc3cc(N4CCO[C@@H](C)C4)ccc3o2)c2cc(NC(=O)[C@@H]3C[C@@H]3CF)ncc12 ; CNc1ncc(-c2nc3cc(N4CCO[C@@H](C)C4)ccc3o2)c2cc(NC(=O)[C@@H]3C[C@@H]3C(F)F)ncc12 ; CNc1ncc(-c2nc3cc(N4CCO[C@@H](C)C4)ccc3o2)c2cc(NC(=O)[C@H]3C[C@H]3C(F)F)ncc12 ; CNc1ncc(-c2nc3cc(N4CCO[C@@H](C)C4)ccc3o2)c2cc(NC(=O)[C@@H]3C[C@@H]3C(F)F)ncc12]

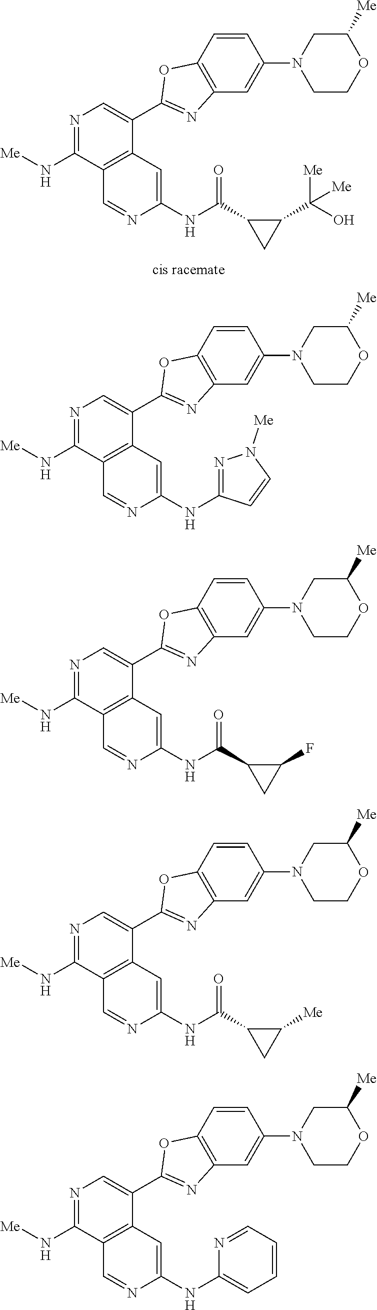 [SMILES: CNc1ncc(-c2nc3cc(N4CCO[C@@H](C)C4)ccc3o2)c2cc(NC(=O)[C@H]3C[C@H]3C(C)(C)O)ncc12 ; CNc1ncc(-c2nc3cc(N4CCO[C@@H](C)C4)ccc3o2)c2cc(Nc3ccn(C)n3)ncc12 ; CNc1ncc(-c2nc3cc(N4CCO[C@H](C)C4)ccc3o2)c2cc(NC(=O)[C@@H]3C[C@@H]3F)ncc12 ; CNc1ncc(-c2nc3cc(N4CCO[C@H](C)C4)ccc3o2)c2cc(NC(=O)[C@H]3C[C@H]3C)ncc12 ; CNc1ncc(-c2nc3cc(N4CCO[C@H](C)C4)ccc3o2)c2cc(Nc3ccccn3)ncc12]

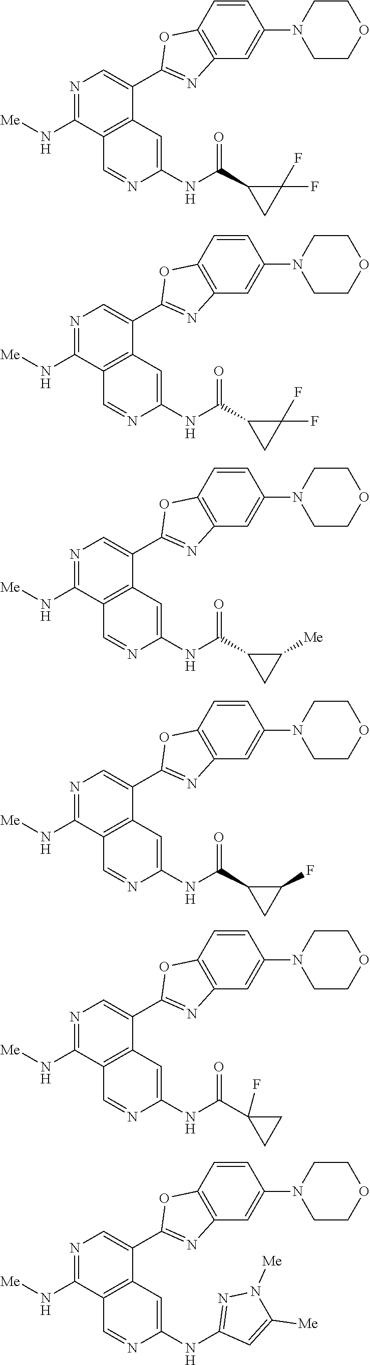

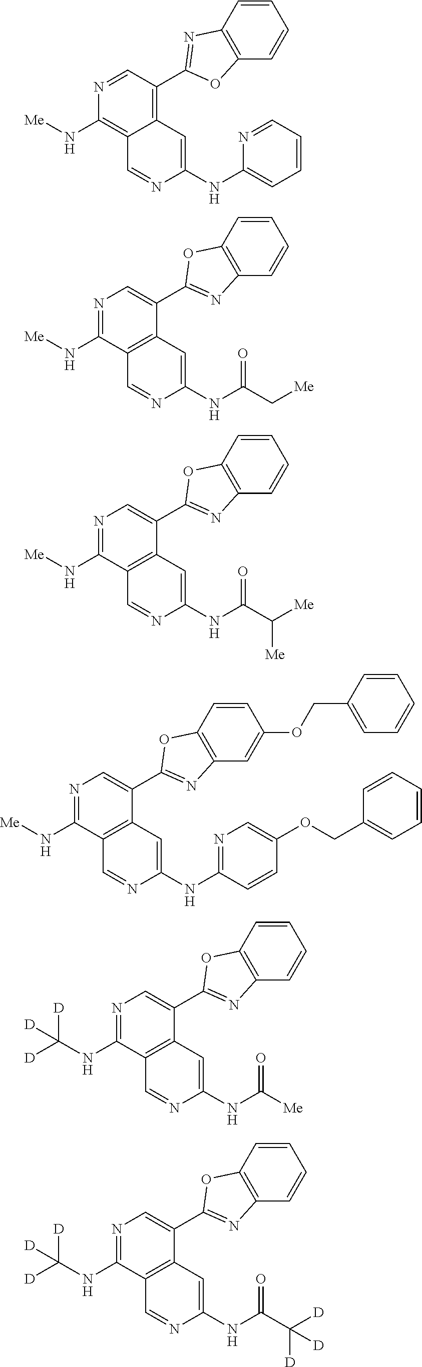

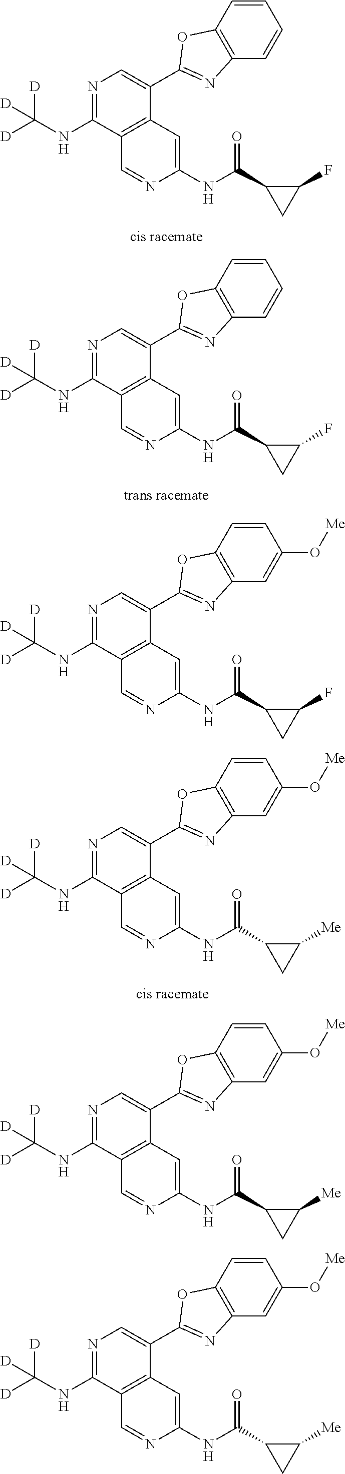

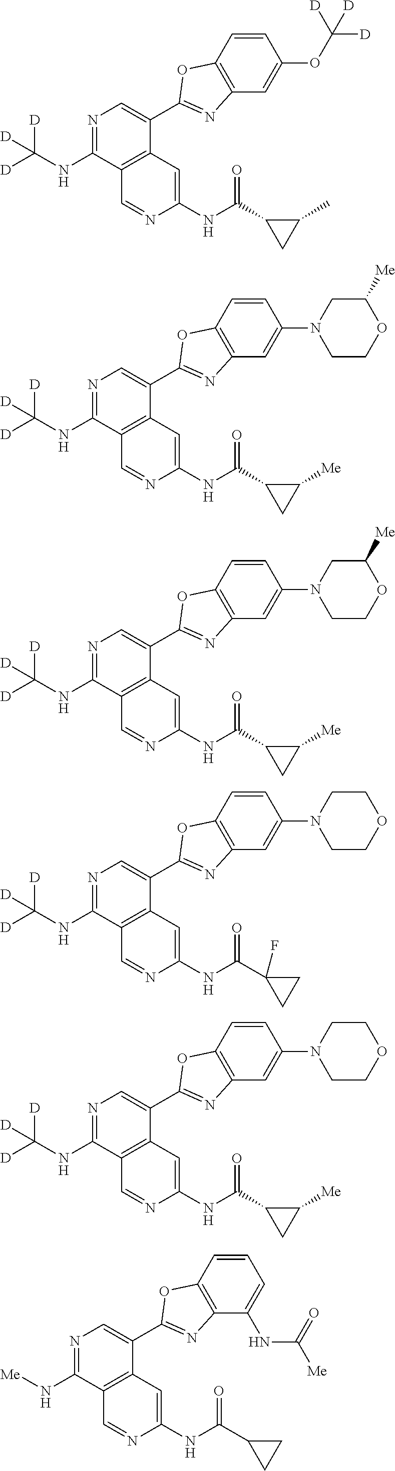

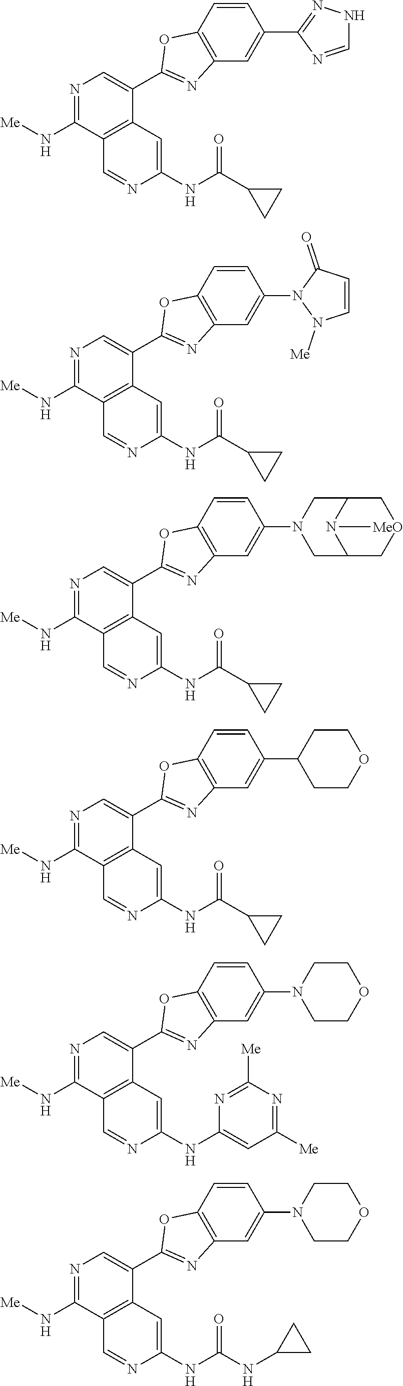

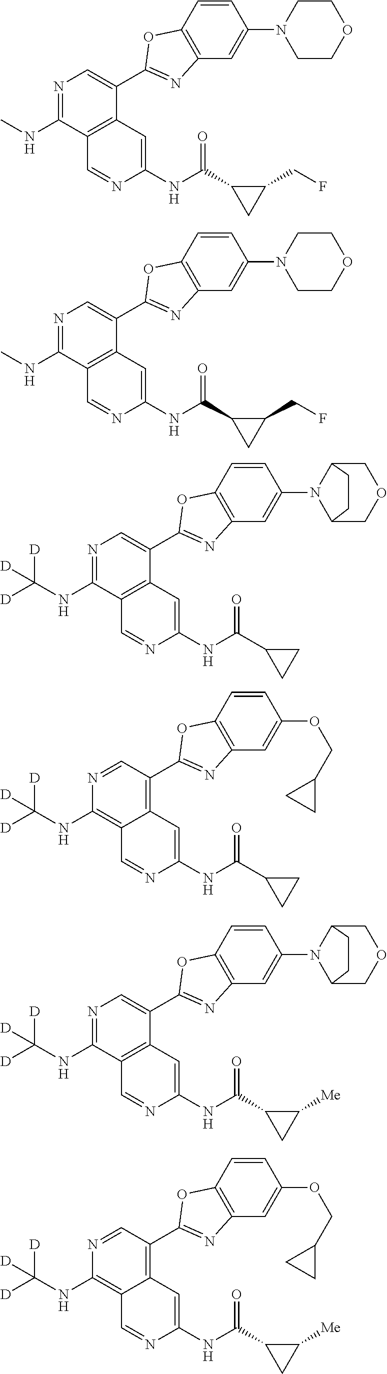

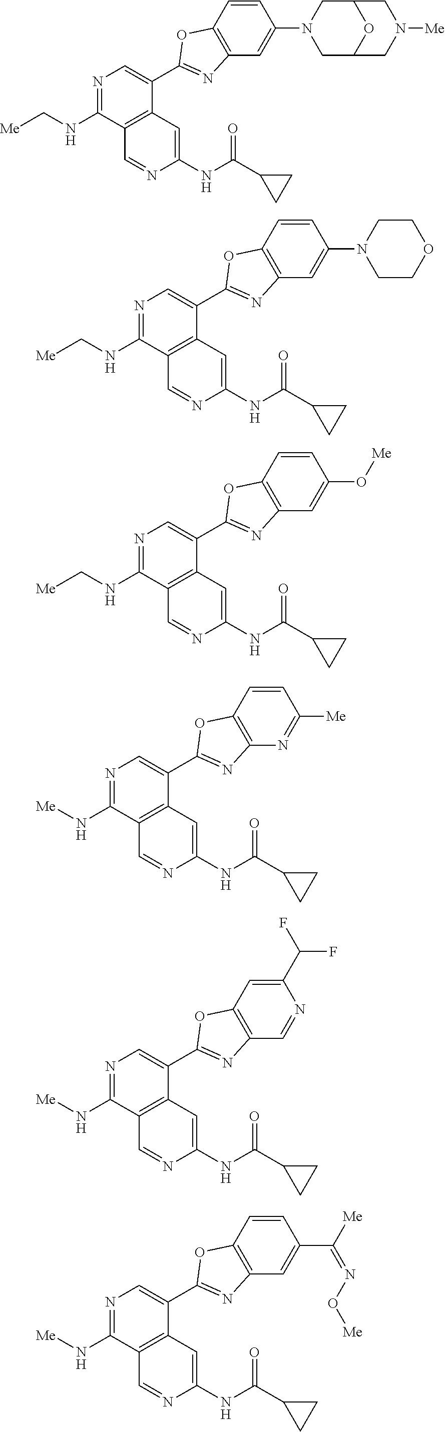

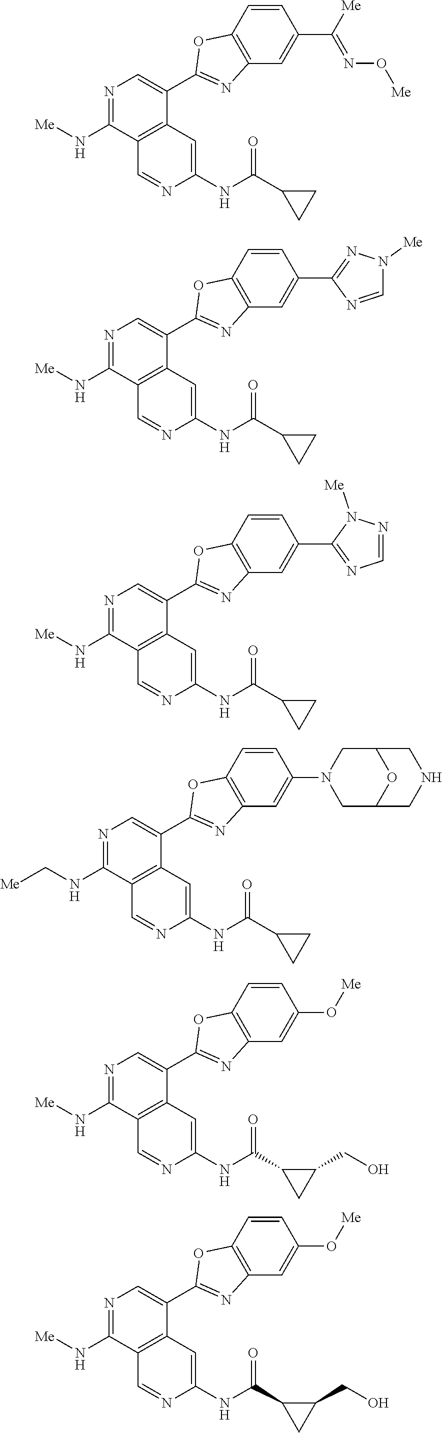

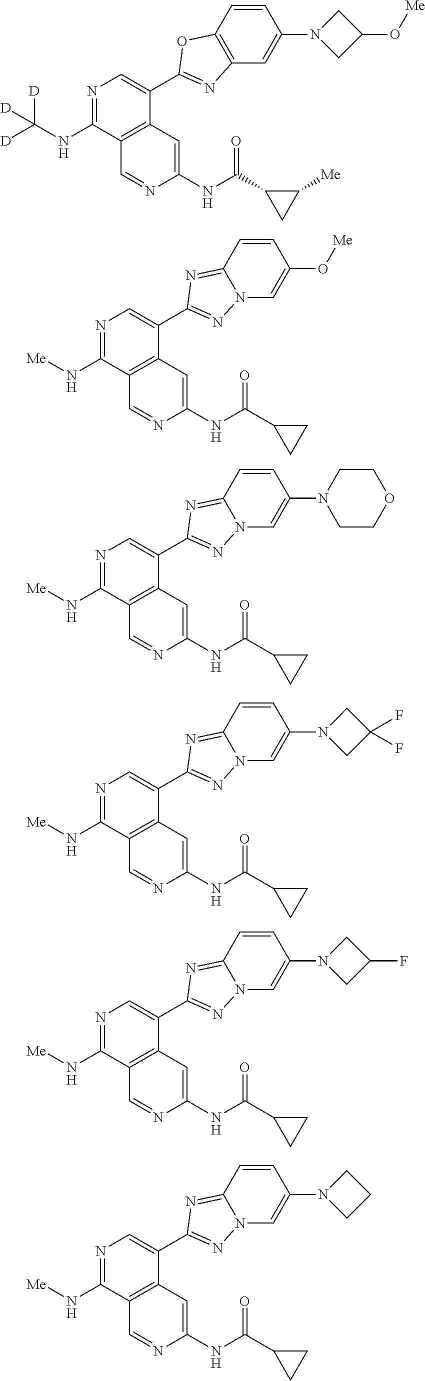

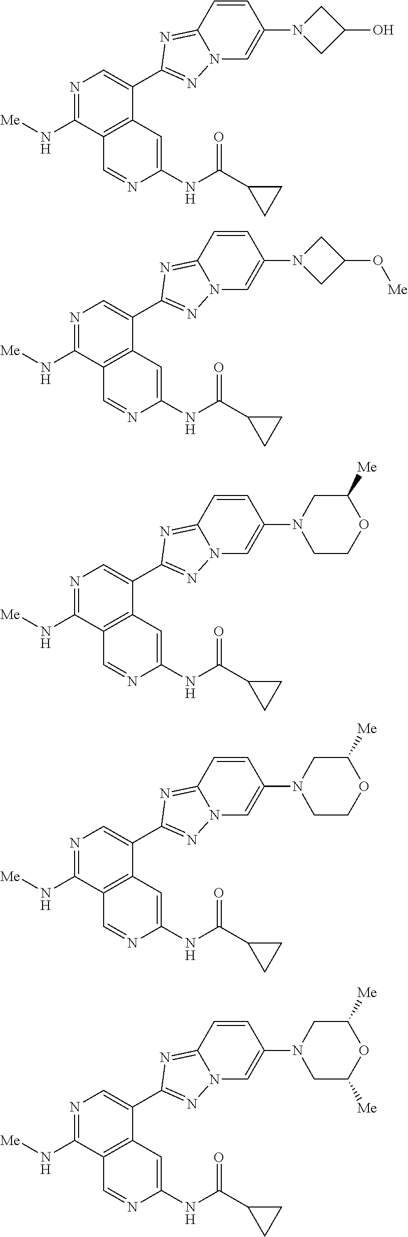

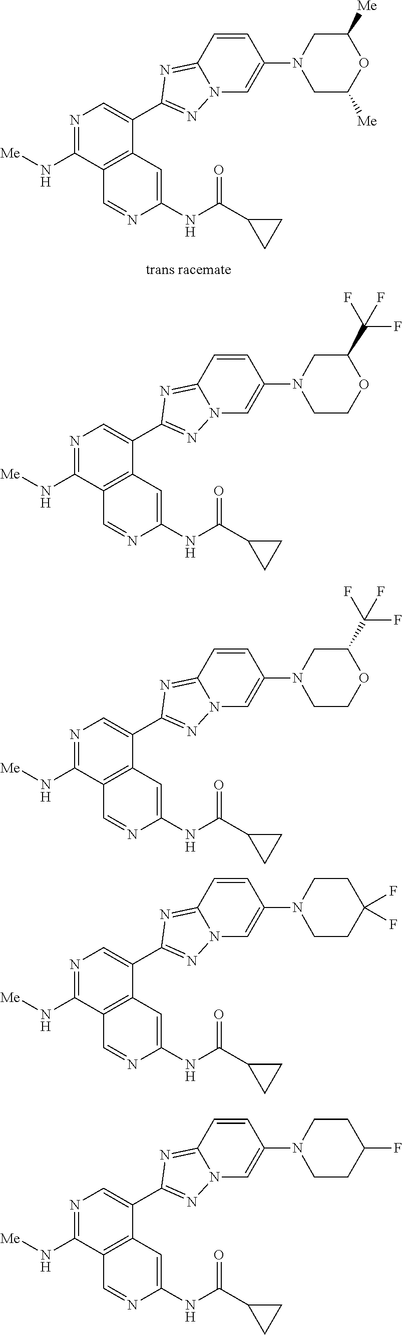

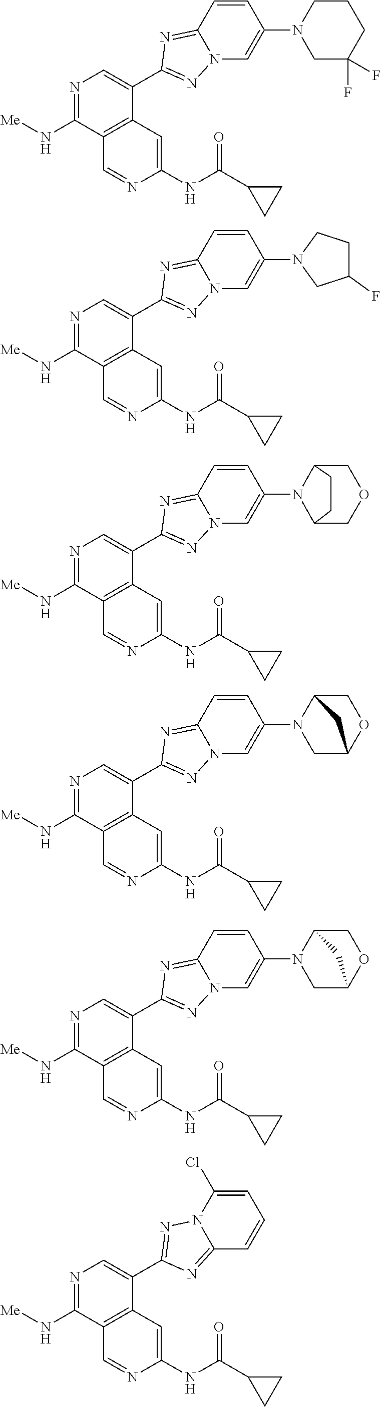

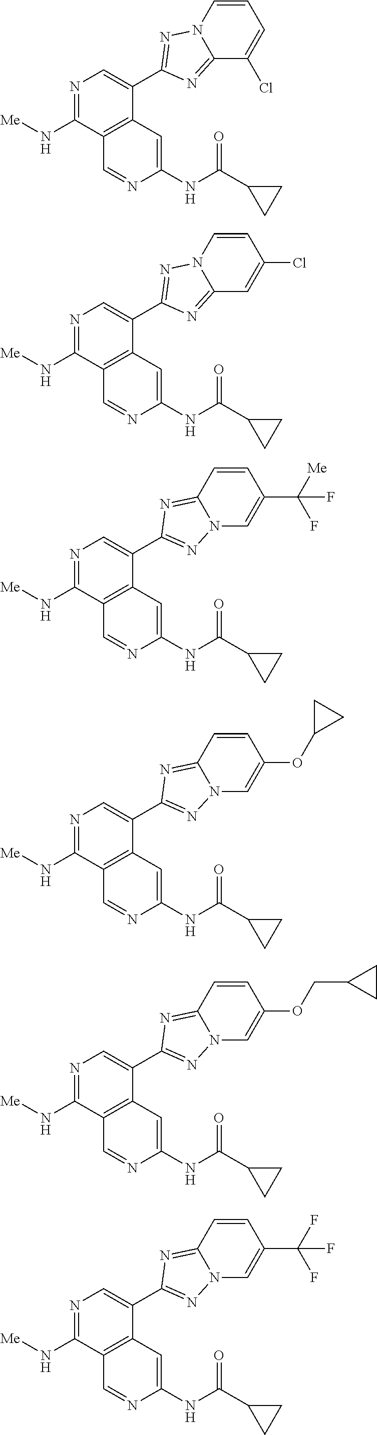

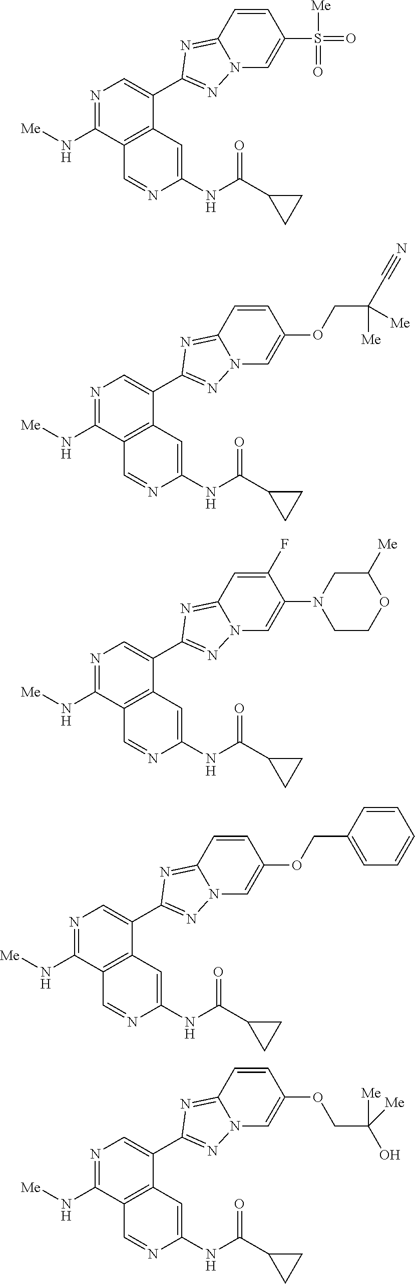

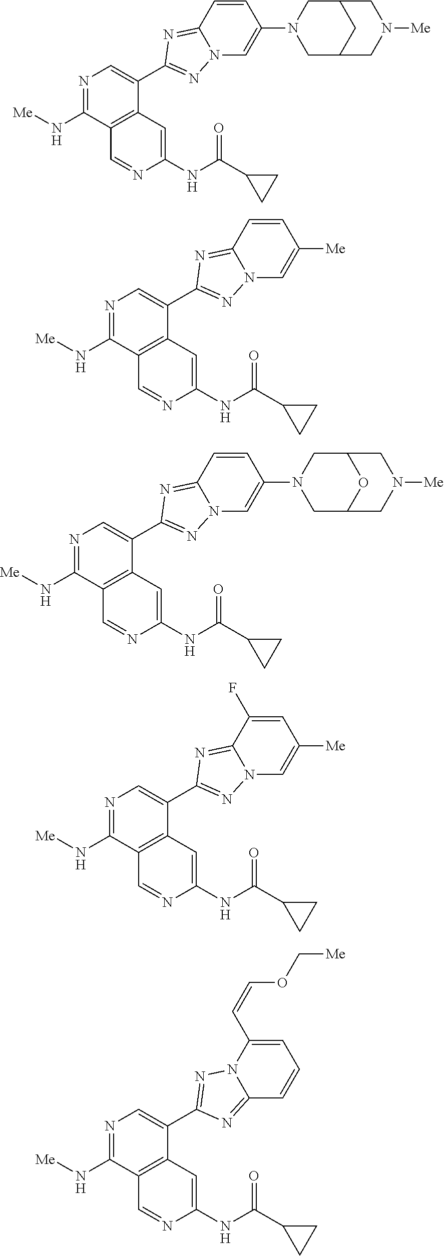

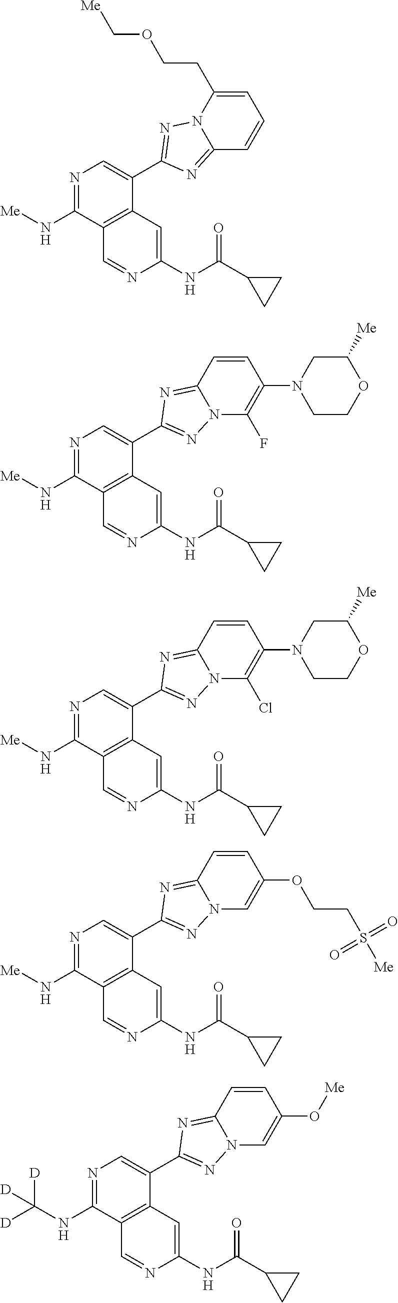

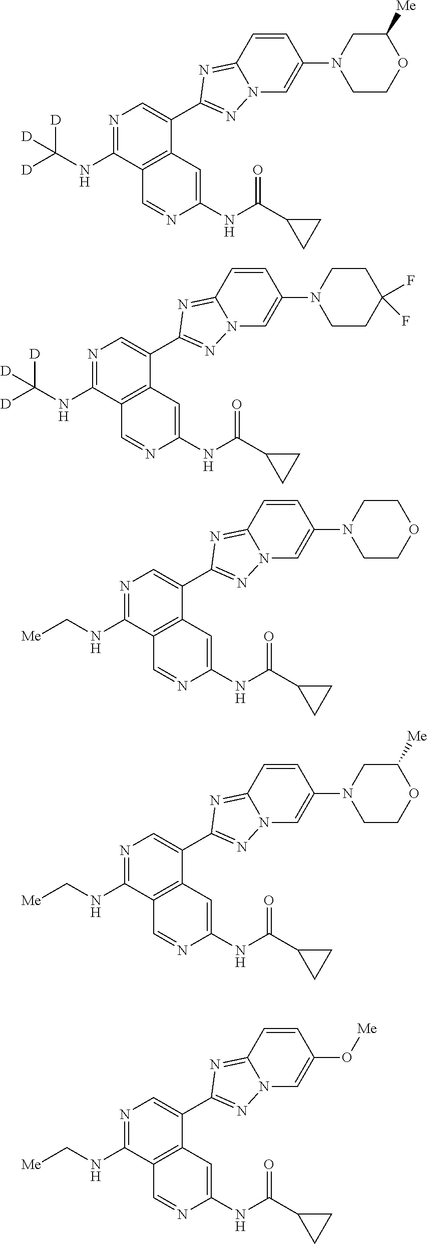

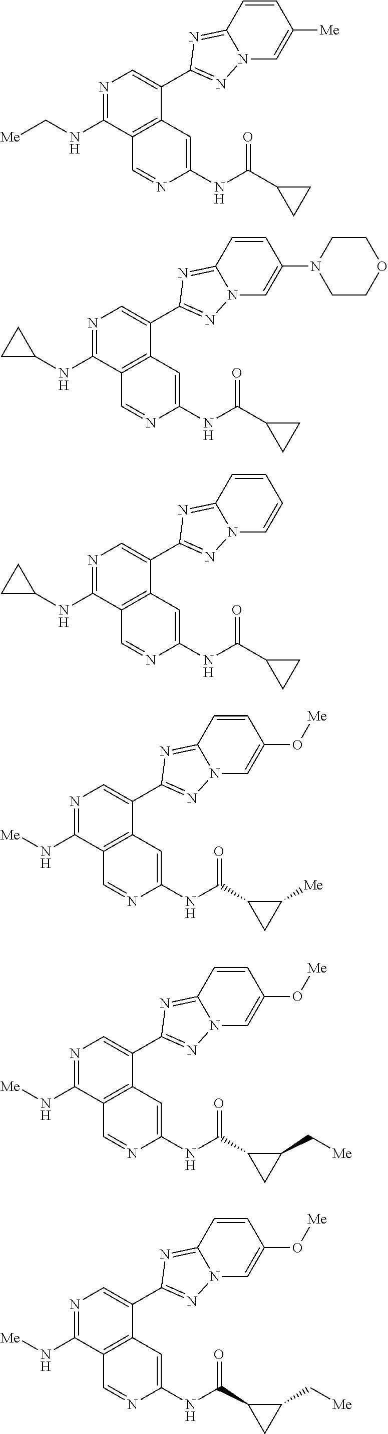

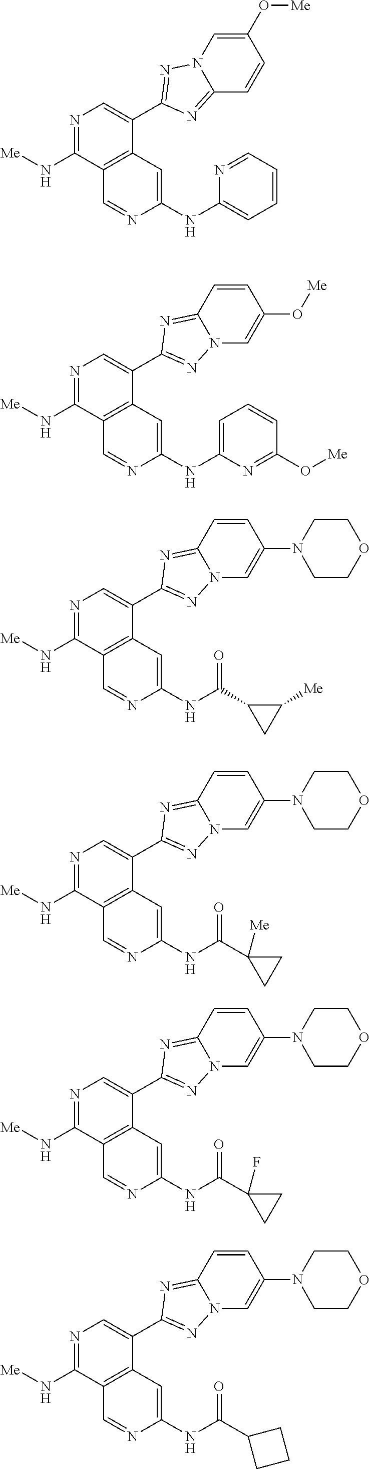

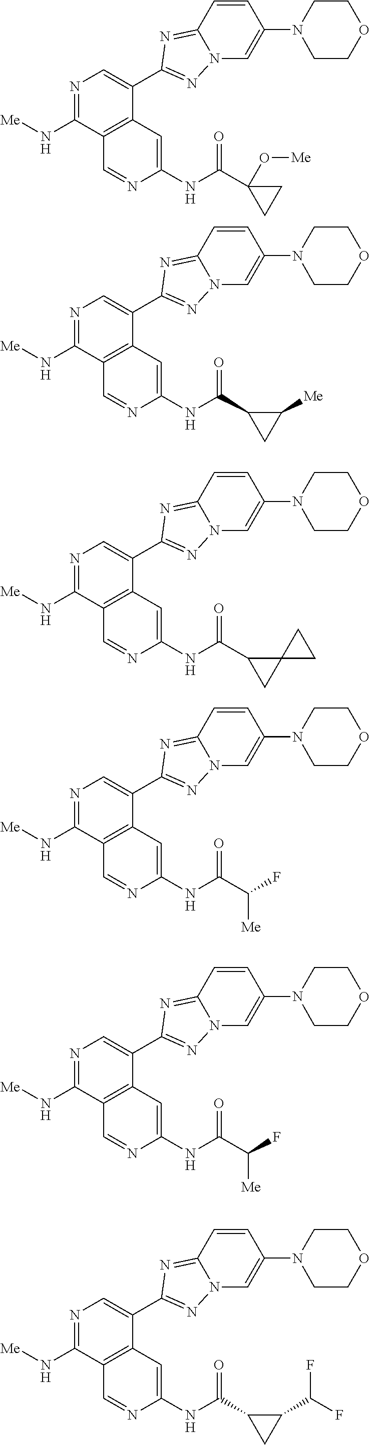

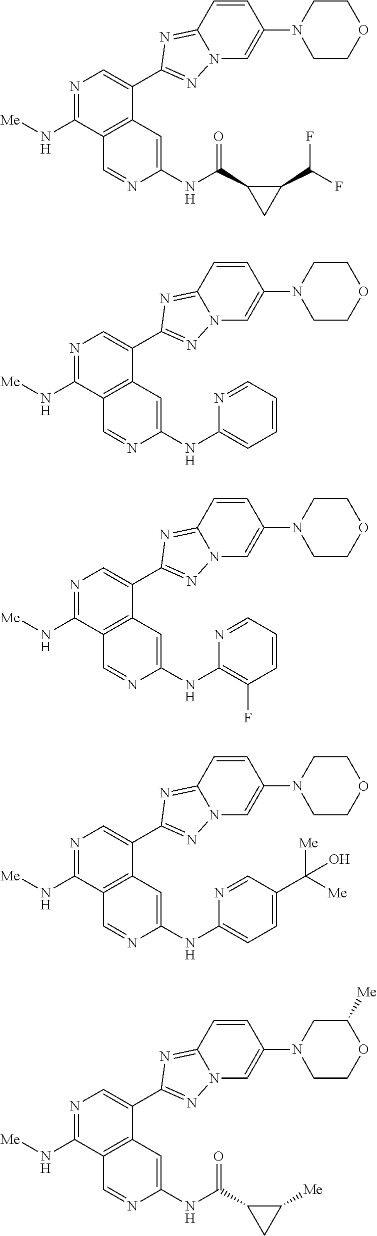

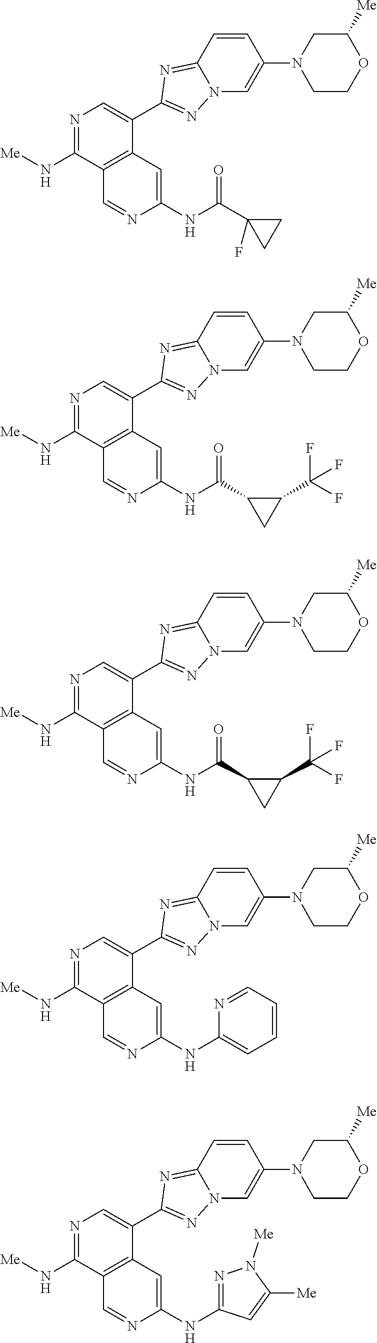 [SMILES: CNc1ncc(-c2nc3ccc(N4CCO[C@@H](C)C4)cn3n2)c2cc(NC(=O)C3(F)CC3)ncc12 ; CNc1ncc(-c2nc3ccc(N4CCO[C@@H](C)C4)cn3n2)c2cc(NC(=O)[C@H]3C[C@H]3C(F)(F)F)ncc12 ; CNc1ncc(-c2nc3ccc(N4CCO[C@@H](C)C4)cn3n2)c2cc(NC(=O)[C@@H]3C[C@@H]3C(F)(F)F)ncc12 ; CNc1ncc(-c2nc3ccc(N4CCO[C@@H](C)C4)cn3n2)c2cc(Nc3ccccn3)ncc12 ; CNc1ncc(-c2nc3ccc(N4CCO[C@@H](C)C4)cn3n2)c2cc(Nc3cc(C)n(C)n3)ncc12]

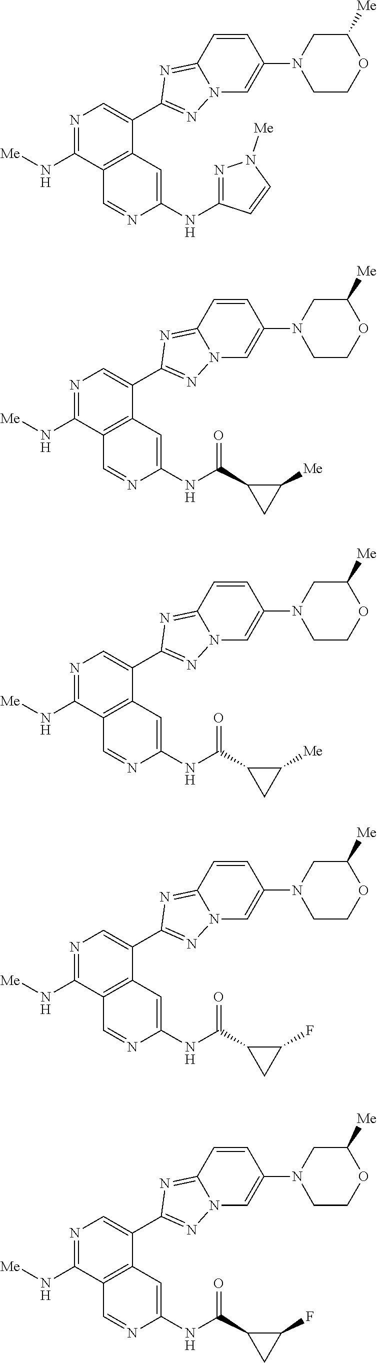 [SMILES: CNc1ncc(-c2nc3ccc(N4CCO[C@@H](C)C4)cn3n2)c2cc(Nc3ccn(C)n3)ncc12 ; CNc1ncc(-c2nc3ccc(N4CCO[C@H](C)C4)cn3n2)c2cc(NC(=O)[C@@H]3C[C@@H]3C)ncc12 ; CNc1ncc(-c2nc3ccc(N4CCO[C@H](C)C4)cn3n2)c2cc(NC(=O)[C@H]3C[C@H]3C)ncc12 ; CNc1ncc(-c2nc3ccc(N4CCO[C@H](C)C4)cn3n2)c2cc(NC(=O)[C@H]3C[C@H]3F)ncc12 ; CNc1ncc(-c2nc3ccc(N4CCO[C@H](C)C4)cn3n2)c2cc(NC(=O)[C@@H]3C[C@@H]3F)ncc12]

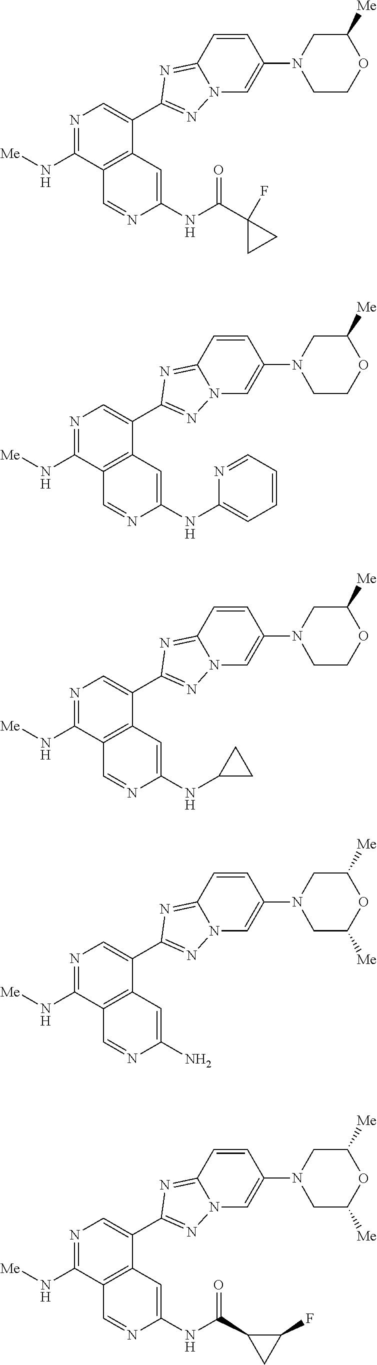 [SMILES: CNc1ncc(-c2nc3ccc(N4CCO[C@H](C)C4)cn3n2)c2cc(NC(=O)C3(F)CC3)ncc12 ; CNc1ncc(-c2nc3ccc(N4CCO[C@H](C)C4)cn3n2)c2cc(Nc3ccccn3)ncc12 ; CNc1ncc(-c2nc3ccc(N4CCO[C@H](C)C4)cn3n2)c2cc(NC3CC3)ncc12 ; CNc1ncc(-c2nc3ccc(N4C[C@@H](C)O[C@@H](C)C4)cn3n2)c2cc(N)ncc12 ; CNc1ncc(-c2nc3ccc(N4C[C@@H](C)O[C@@H](C)C4)cn3n2)c2cc(NC(=O)[C@@H]3C[C@@H]3F)ncc12]

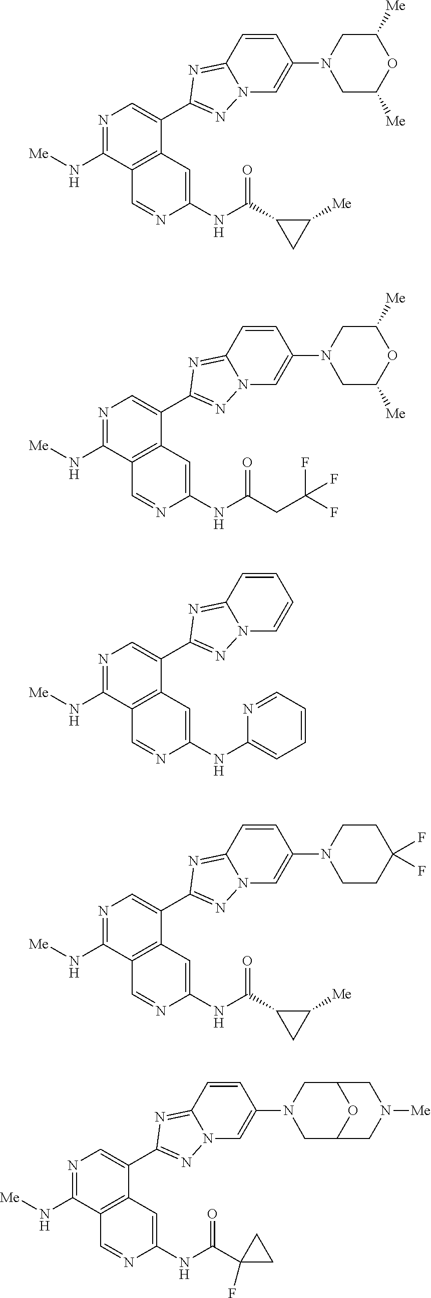

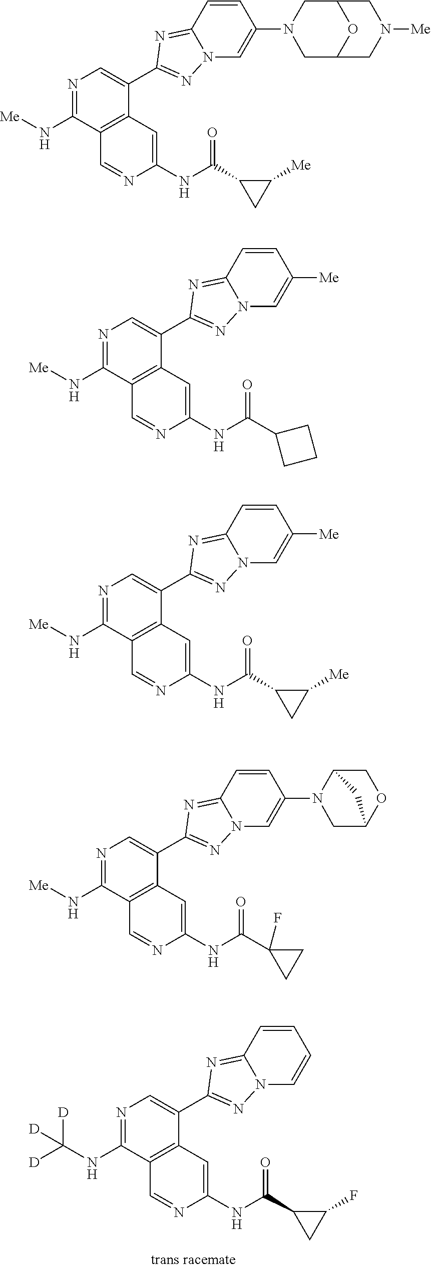

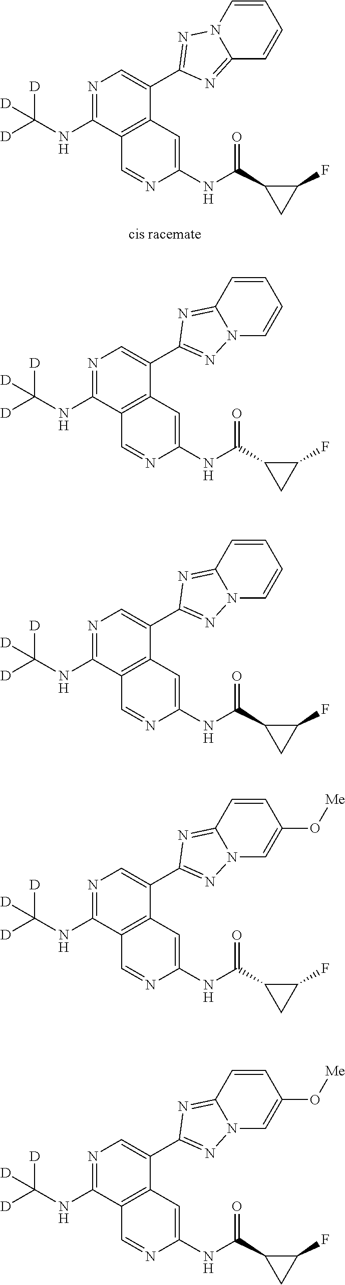 [SMILES: [2H]C([2H])([2H])Nc1ncc(-c2nc3ccccn3n2)c2cc(NC(=O)[C@@H]3C[C@@H]3F)ncc12 ; [2H]C([2H])([2H])Nc1ncc(-c2nc3ccccn3n2)c2cc(NC(=O)[C@H]3C[C@H]3F)ncc12 ; [2H]C([2H])([2H])Nc1ncc(-c2nc3ccccn3n2)c2cc(NC(=O)[C@@H]3C[C@@H]3F)ncc12 ; [2H]C([2H])([2H])Nc1ncc(-c2nc3ccc(OC)cn3n2)c2cc(NC(=O)[C@H]3C[C@H]3F)ncc12 ; [2H]C([2H])([2H])Nc1ncc(-c2nc3ccc(OC)cn3n2)c2cc(NC(=O)[C@@H]3C[C@@H]3F)ncc12]

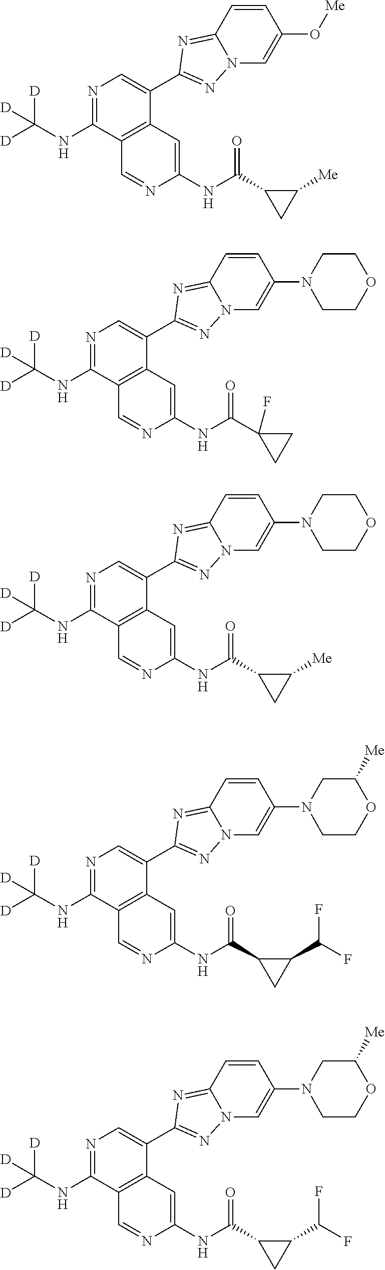

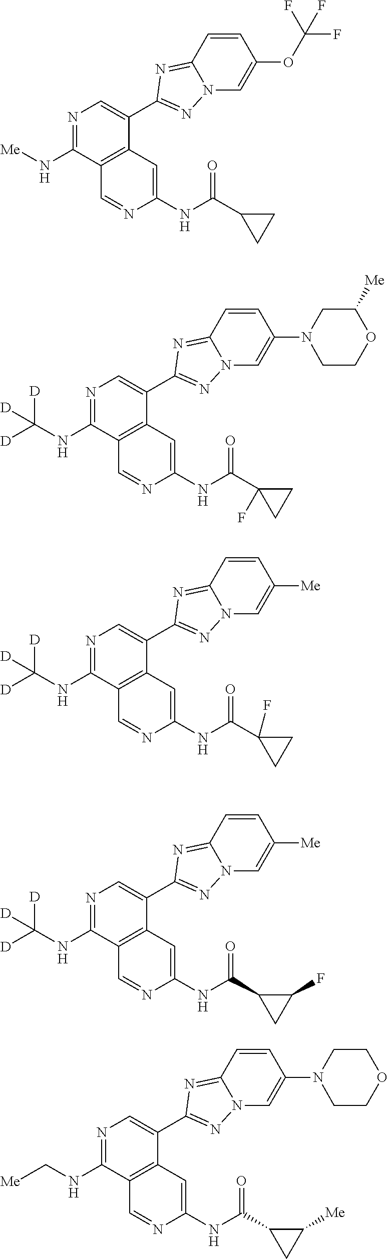

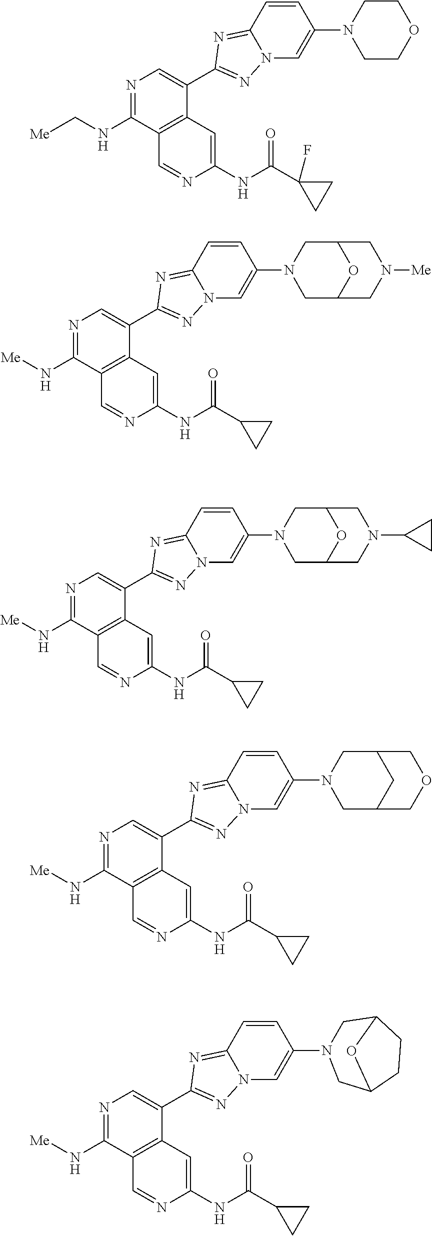

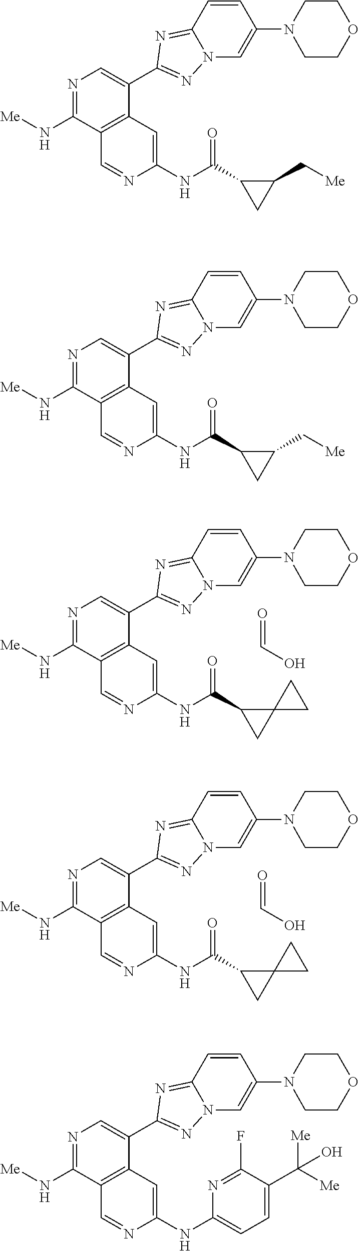

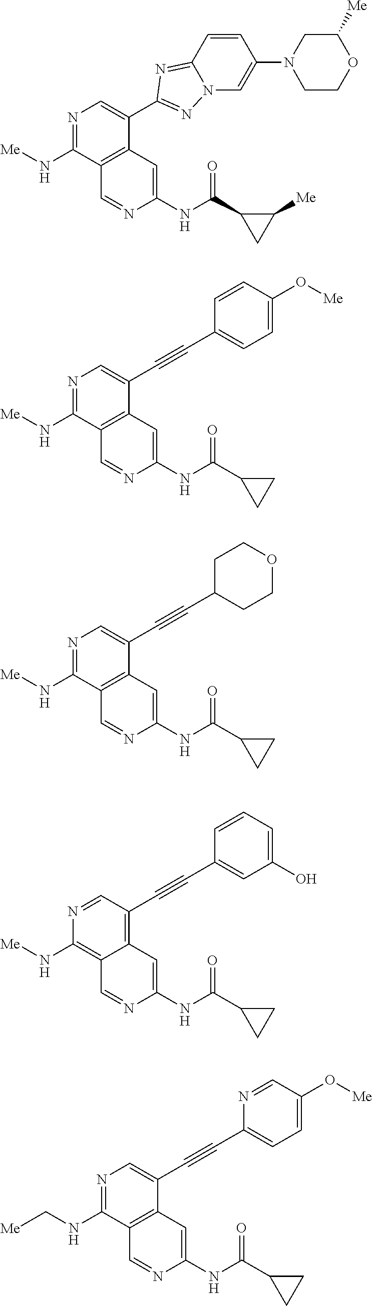

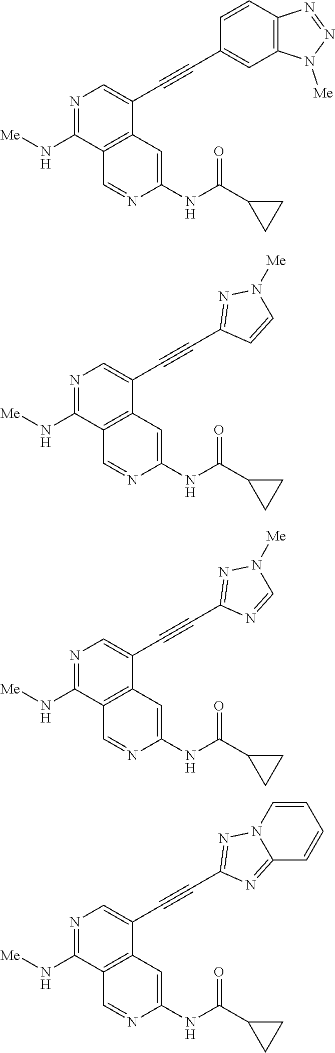

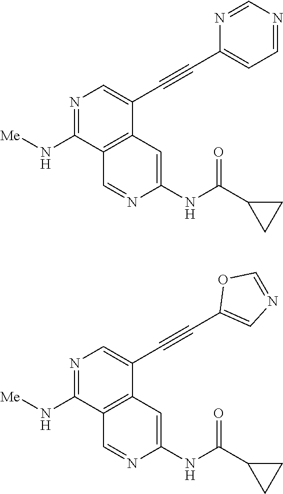

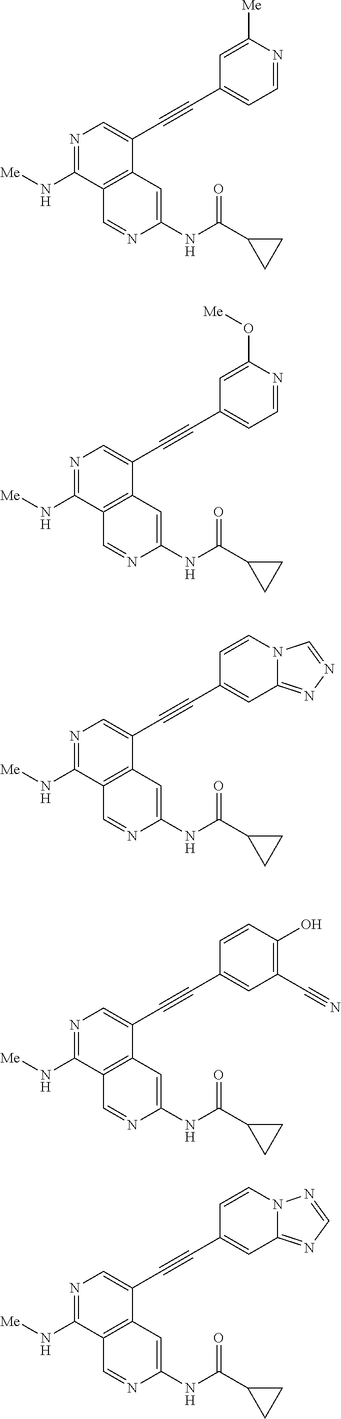

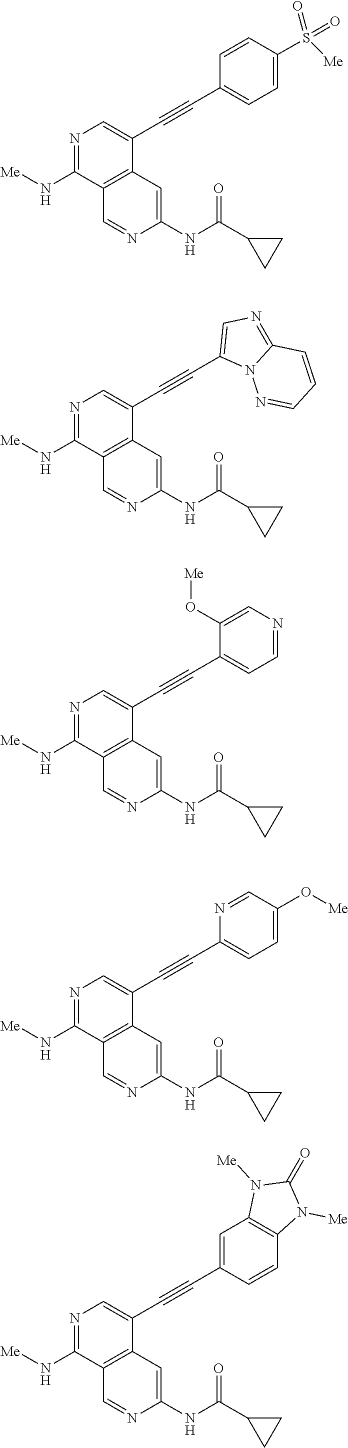 [SMILES: CNc1ncc(C#Cc2ccc(S(C)(=O)=O)cc2)c2cc(NC(=O)C3CC3)ncc12 ; CNc1ncc(C#Cc2cnc3cccnn23)c2cc(NC(=O)C3CC3)ncc12 ; CNc1ncc(C#Cc2ccncc2OC)c2cc(NC(=O)C3CC3)ncc12 ; CNc1ncc(C#Cc2ccc(OC)cn2)c2cc(NC(=O)C3CC3)ncc12 ; CNc1ncc(C#Cc2ccc3c(c2)n(C)c(=O)n3C)c2cc(NC(=O)C3CC3)ncc12]

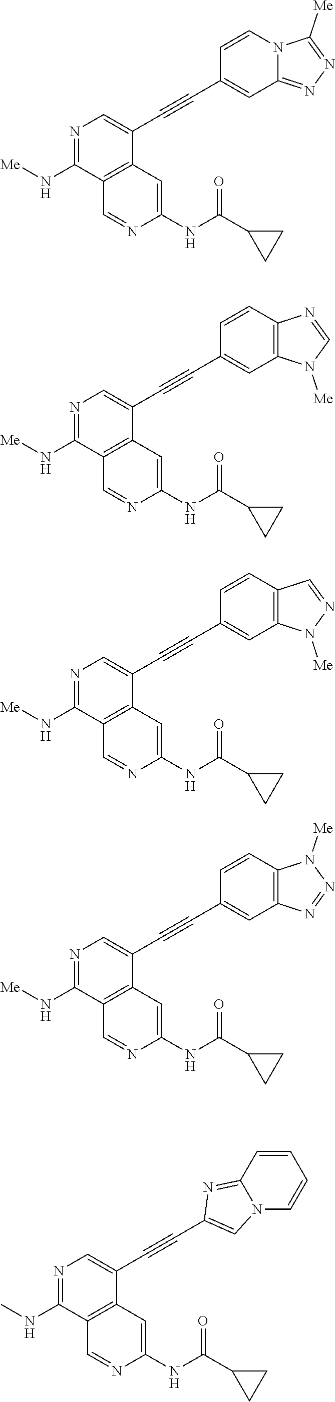

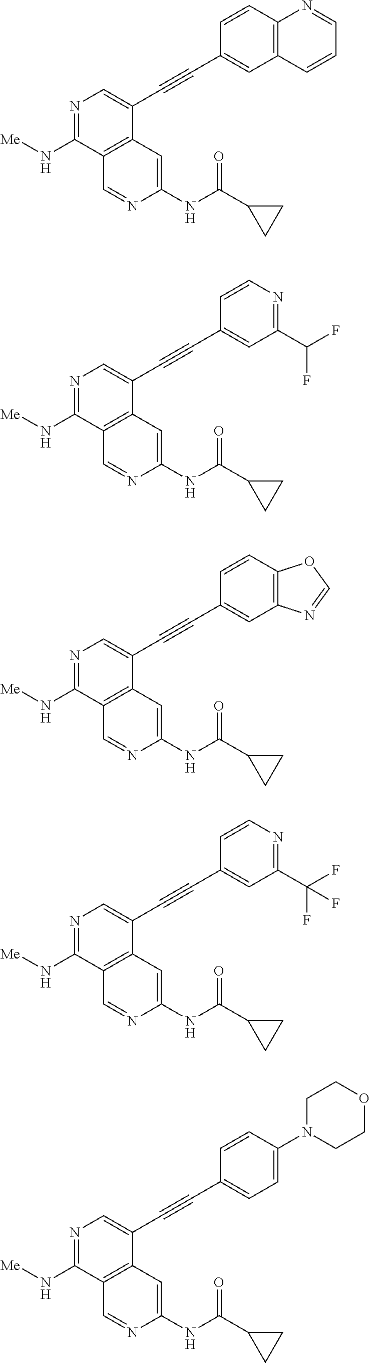

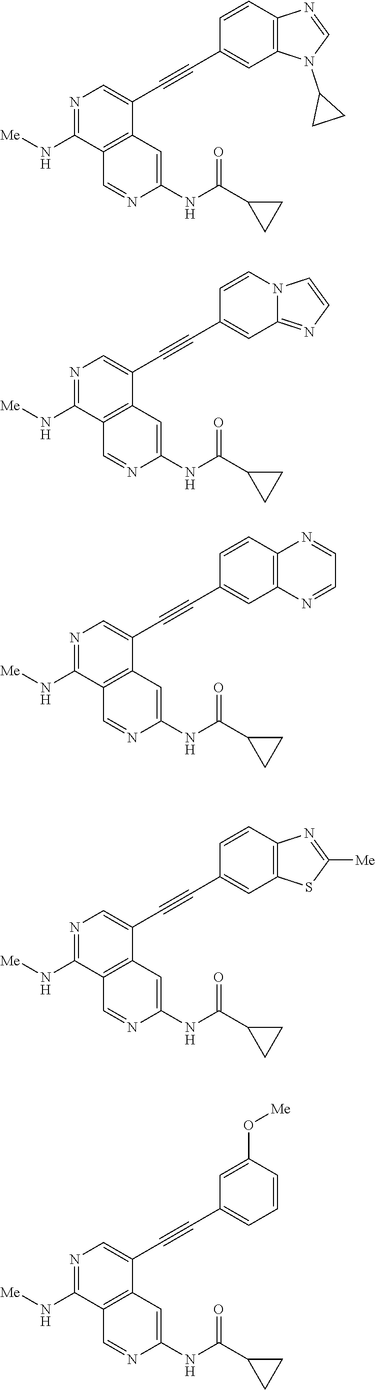

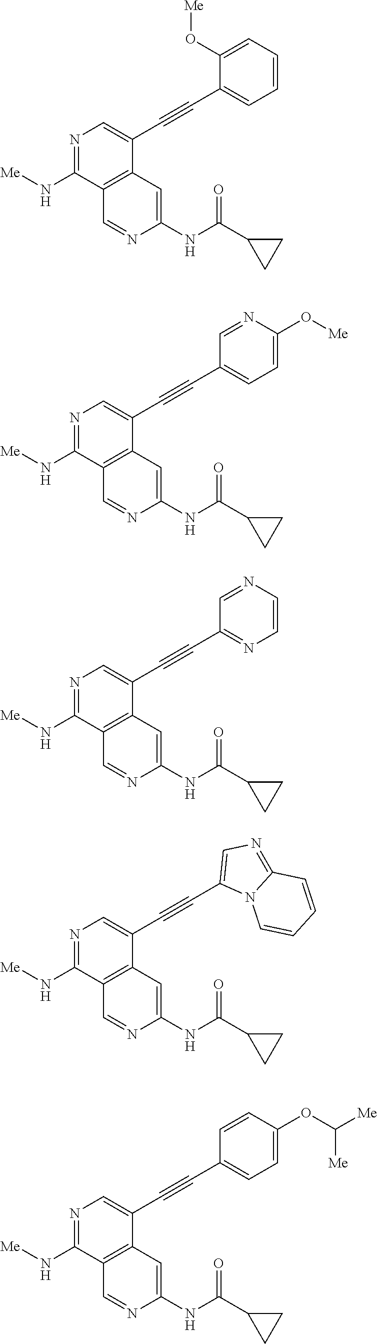

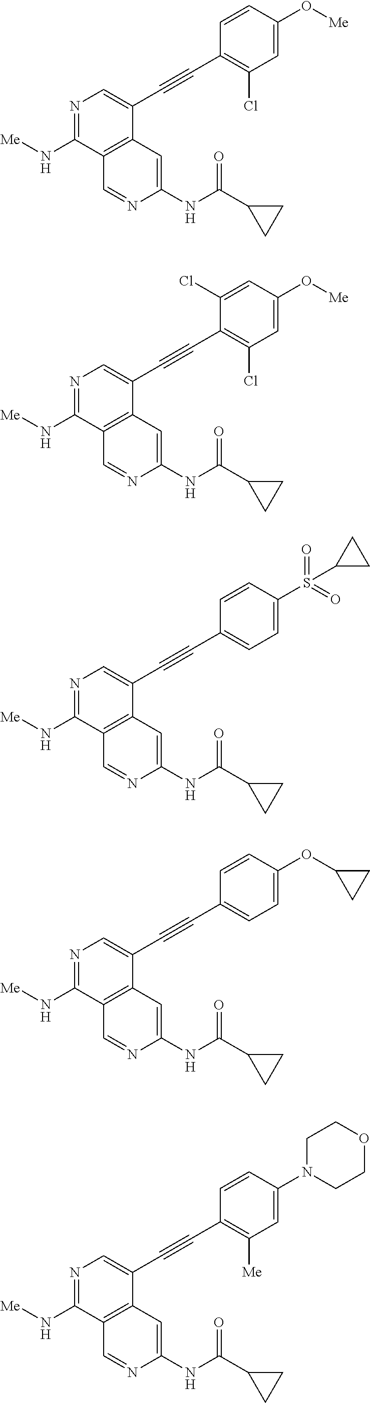

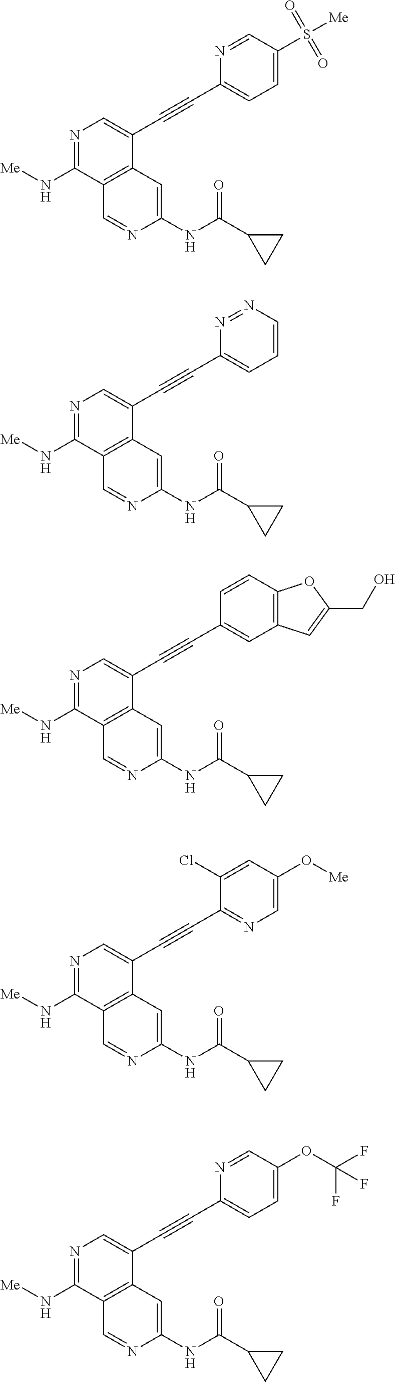

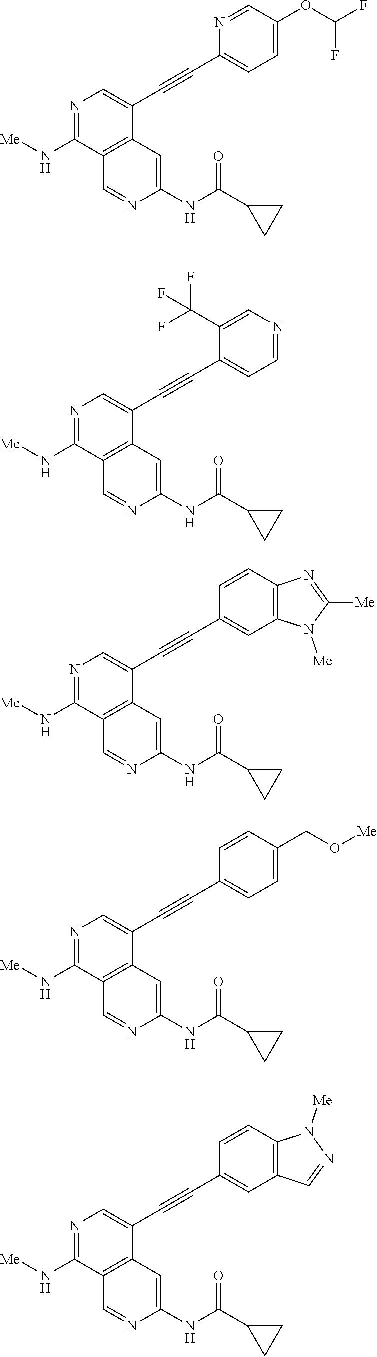

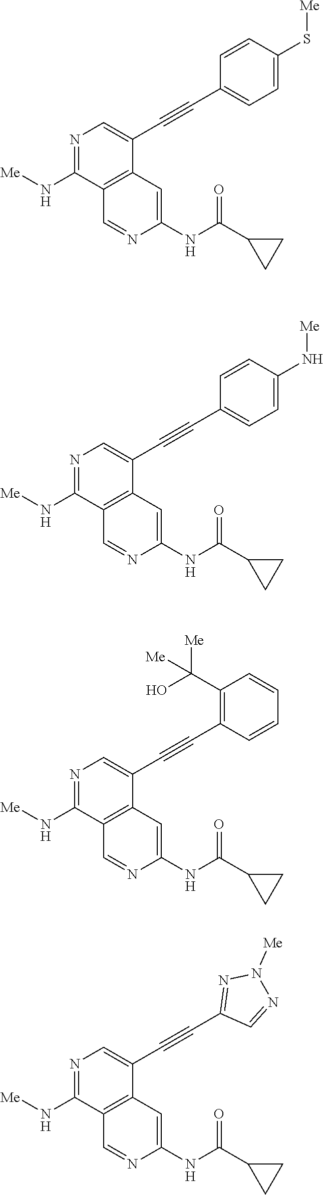

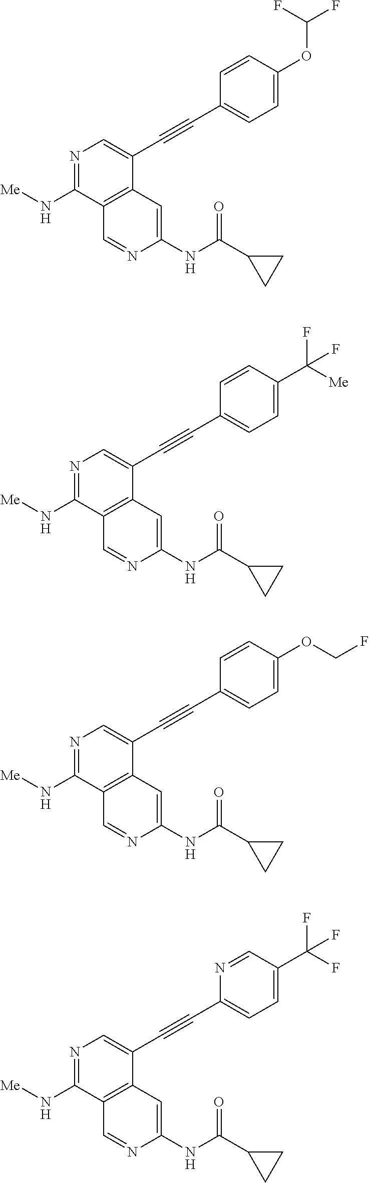 [SMILES: CNc1ncc(C#Cc2ccc(OC(F)F)cc2)c2cc(NC(=O)C3CC3)ncc12 ; CNc1ncc(C#Cc2ccc(C(C)(F)F)cc2)c2cc(NC(=O)C3CC3)ncc12 ; CNc1ncc(C#Cc2ccc(OCF)cc2)c2cc(NC(=O)C3CC3)ncc12 ; CNc1ncc(C#Cc2ccc(C(F)(F)F)cn2)c2cc(NC(=O)C3CC3)ncc12]

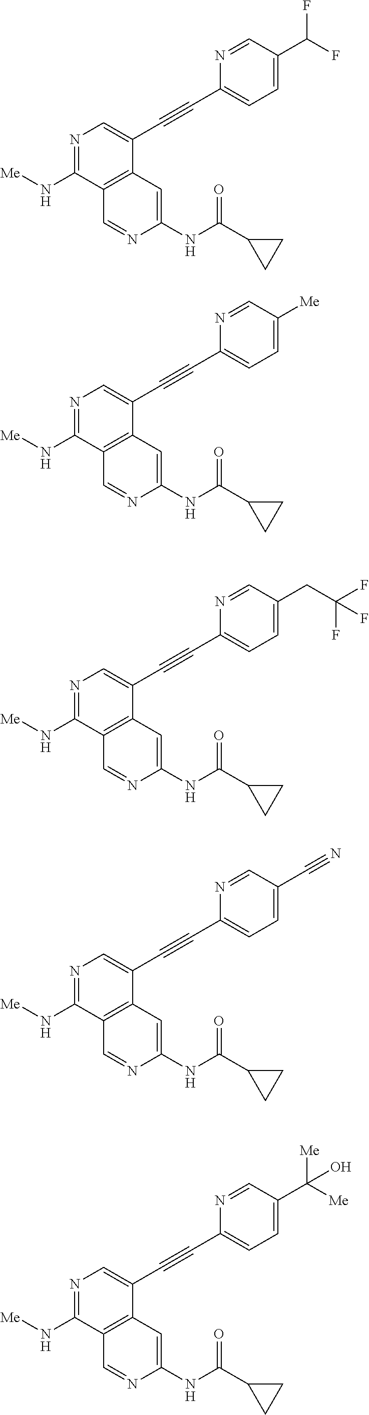

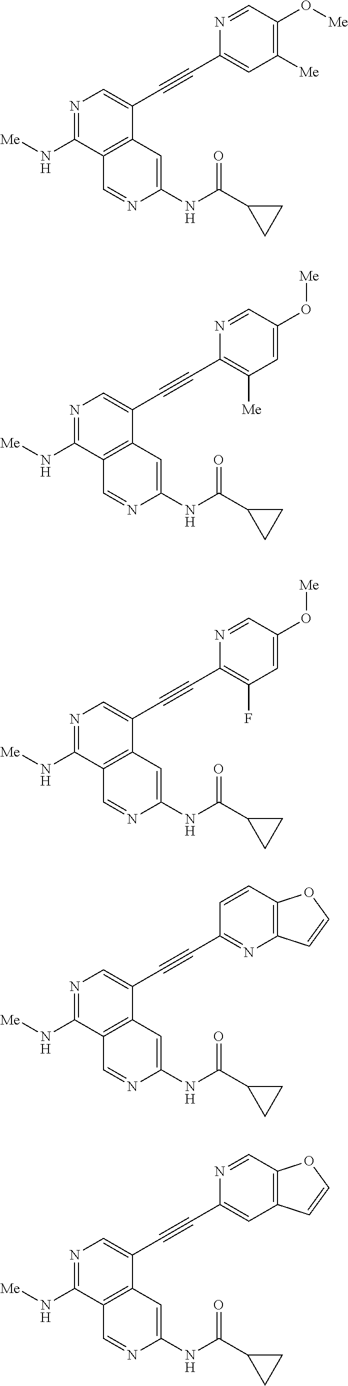

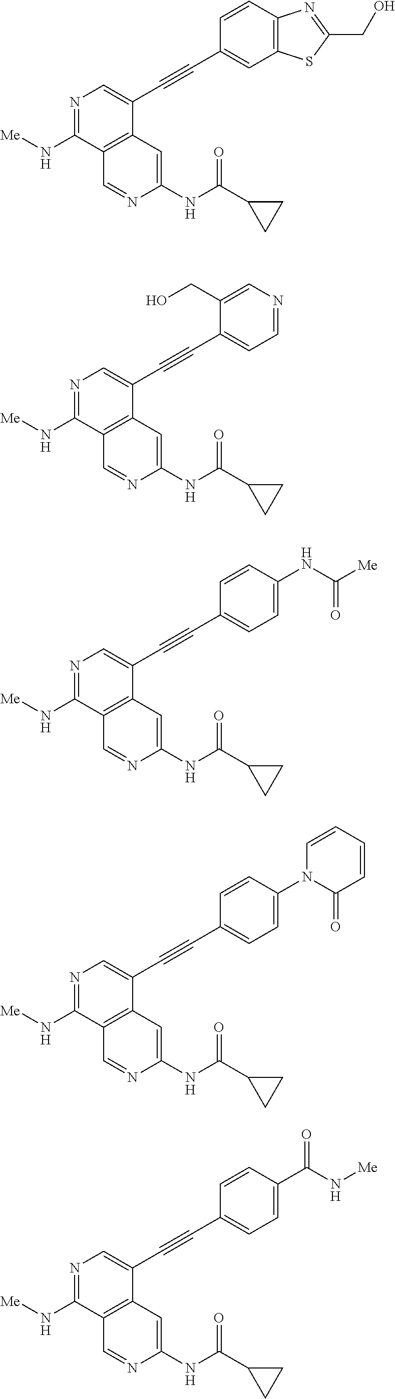 [SMILES: CNc1ncc(C#Cc2ccc3nc(CO)sc3c2)c2cc(NC(=O)C3CC3)ncc12 ; CNc1ncc(C#Cc2ccncc2CO)c2cc(NC(=O)C3CC3)ncc12 ; CNc1ncc(C#Cc2ccc(NC(C)=O)cc2)c2cc(NC(=O)C3CC3)ncc12 ; CNc1ncc(C#Cc2ccc(-n3ccccc3=O)cc2)c2cc(NC(=O)C3CC3)ncc12 ; CNC(=O)c1ccc(C#Cc2cnc(NC)c3cnc(NC(=O)C4CC4)cc23)cc1]

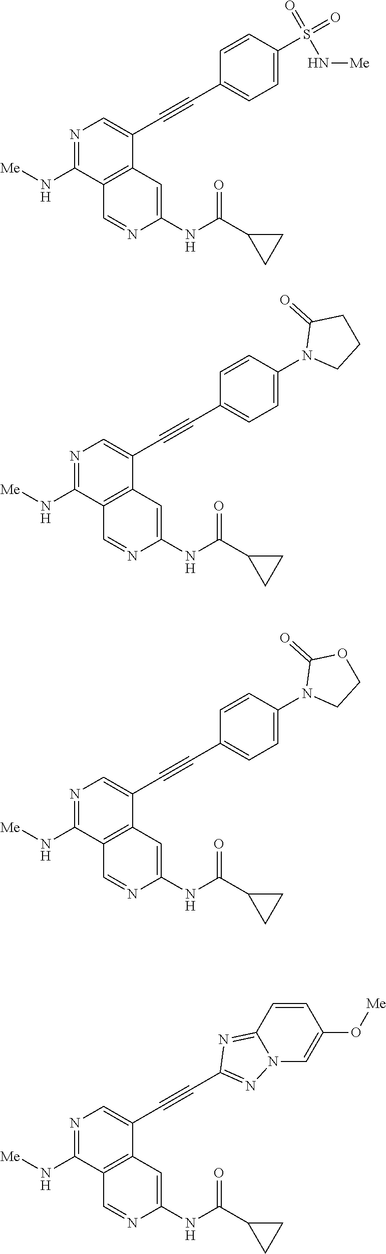

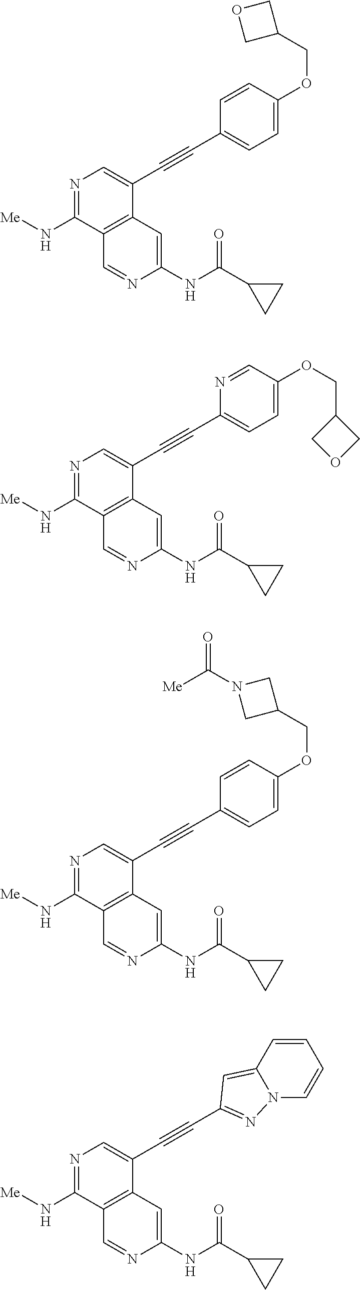 [SMILES: CNc1ncc(C#Cc2ccc(OCC3COC3)cc2)c2cc(NC(=O)C3CC3)ncc12 ; CNc1ncc(C#Cc2ccc(OCC3COC3)cn2)c2cc(NC(=O)C3CC3)ncc12 ; CNc1ncc(C#Cc2ccc(OCC3CN(C(C)=O)C3)cc2)c2cc(NC(=O)C3CC3)ncc12 ; CNc1ncc(C#Cc2cc3ccccn3n2)c2cc(NC(=O)C3CC3)ncc12]

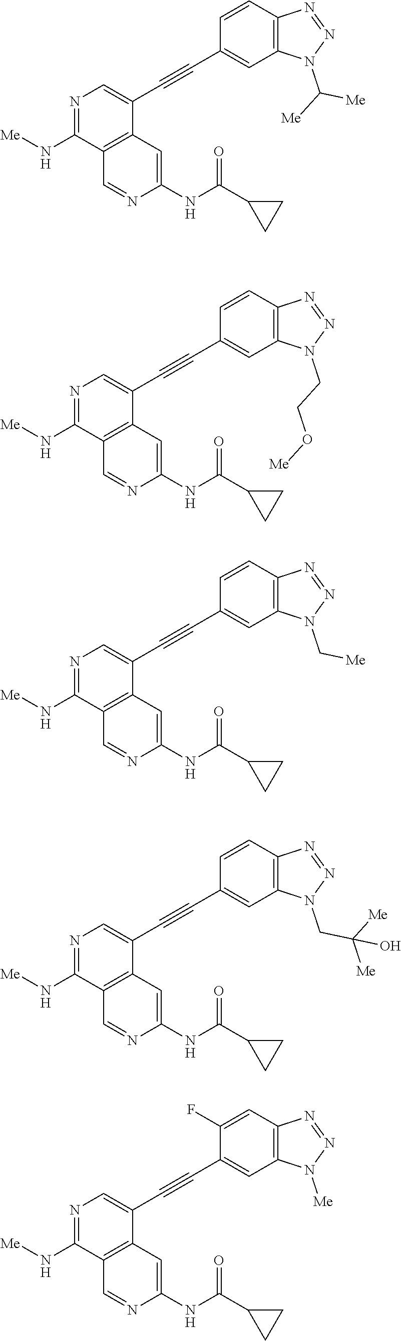

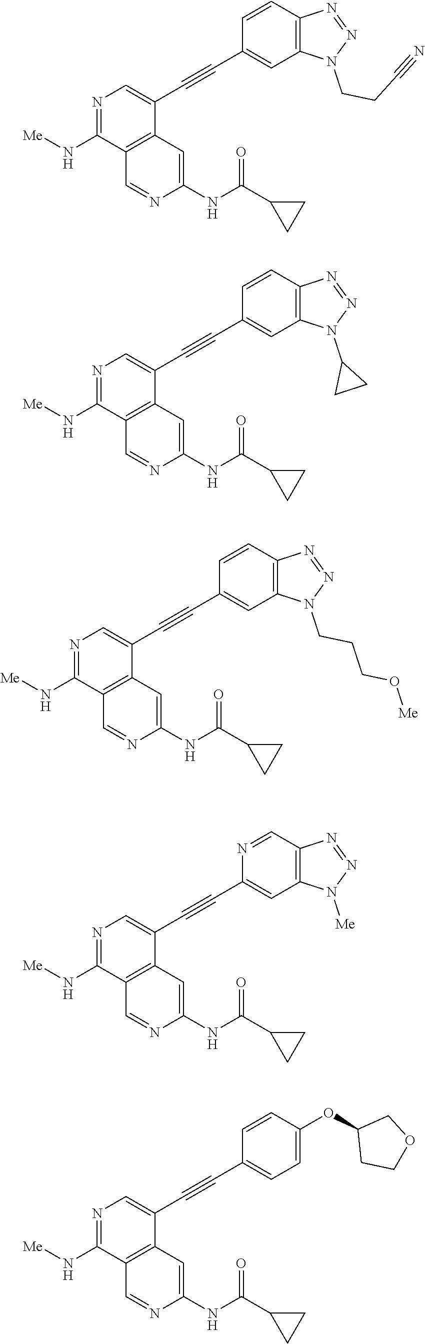

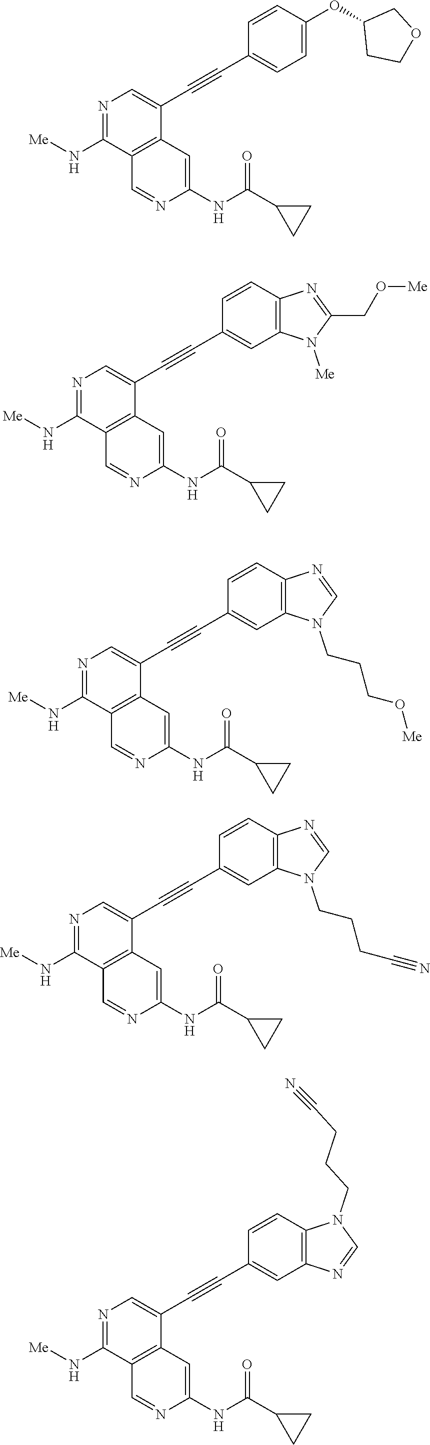

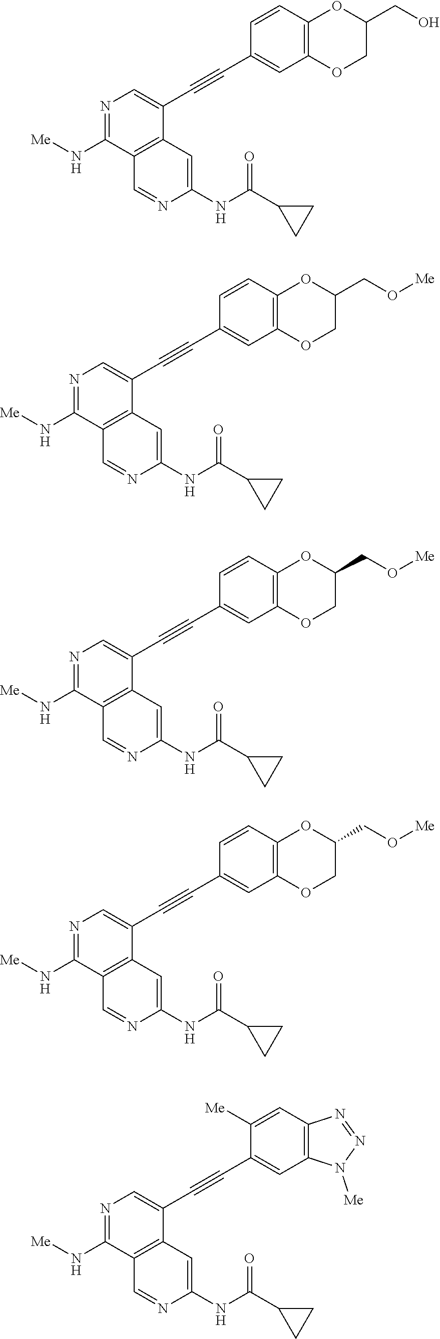

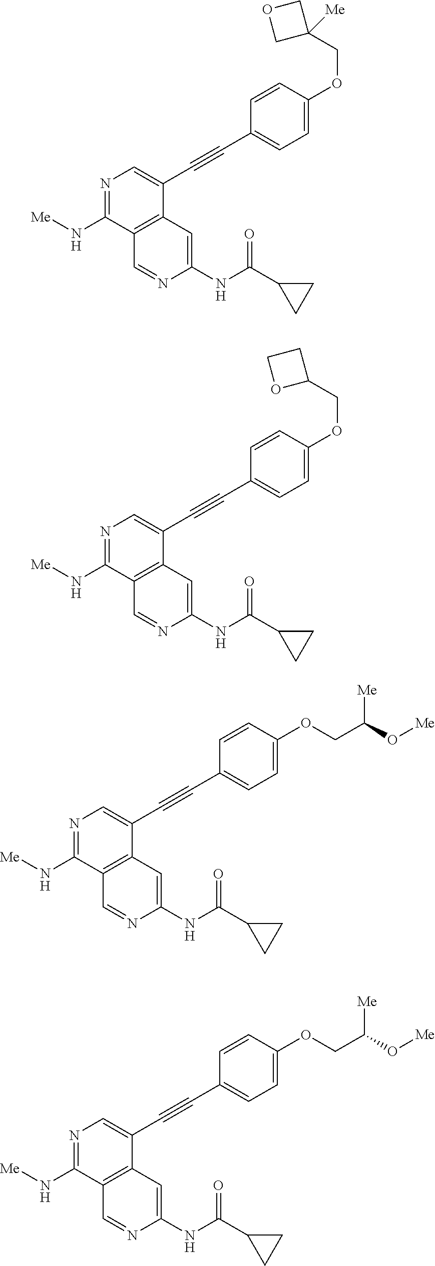

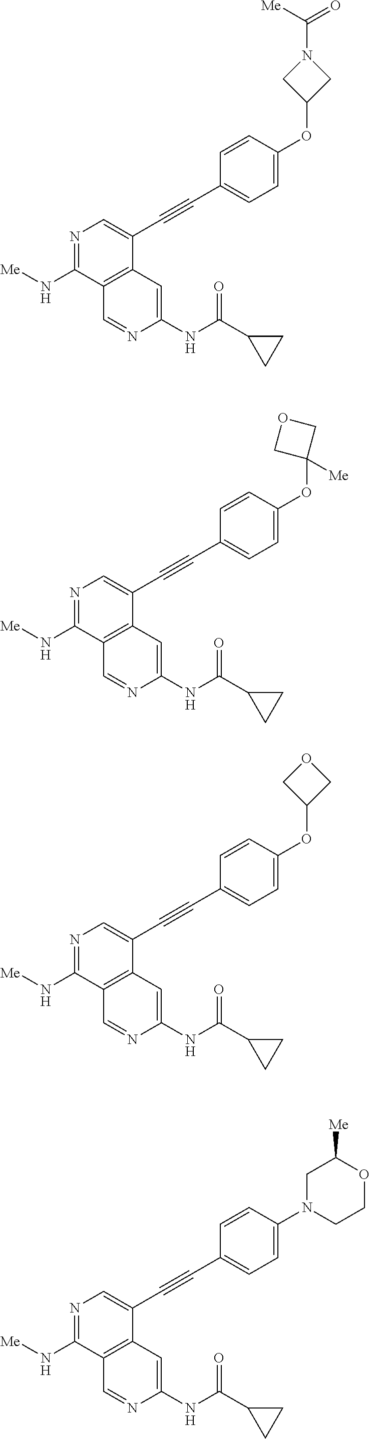 [SMILES: CNc1ncc(C#Cc2ccc(OC3CN(C(C)=O)C3)cc2)c2cc(NC(=O)C3CC3)ncc12 ; CNc1ncc(C#Cc2ccc(OC3(C)COC3)cc2)c2cc(NC(=O)C3CC3)ncc12 ; CNc1ncc(C#Cc2ccc(OC3COC3)cc2)c2cc(NC(=O)C3CC3)ncc12 ; CNc1ncc(C#Cc2ccc(N3CCO[C@H](C)C3)cc2)c2cc(NC(=O)C3CC3)ncc12]

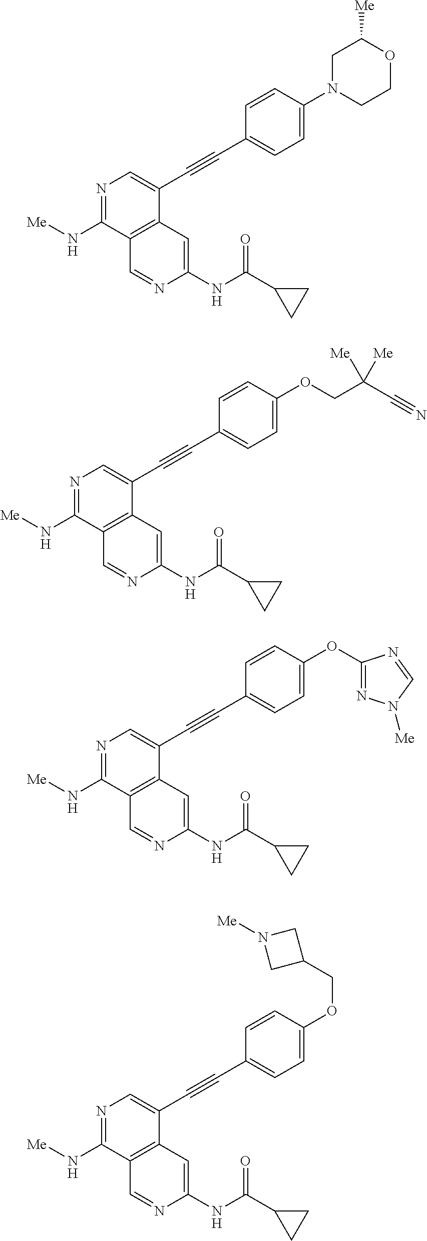

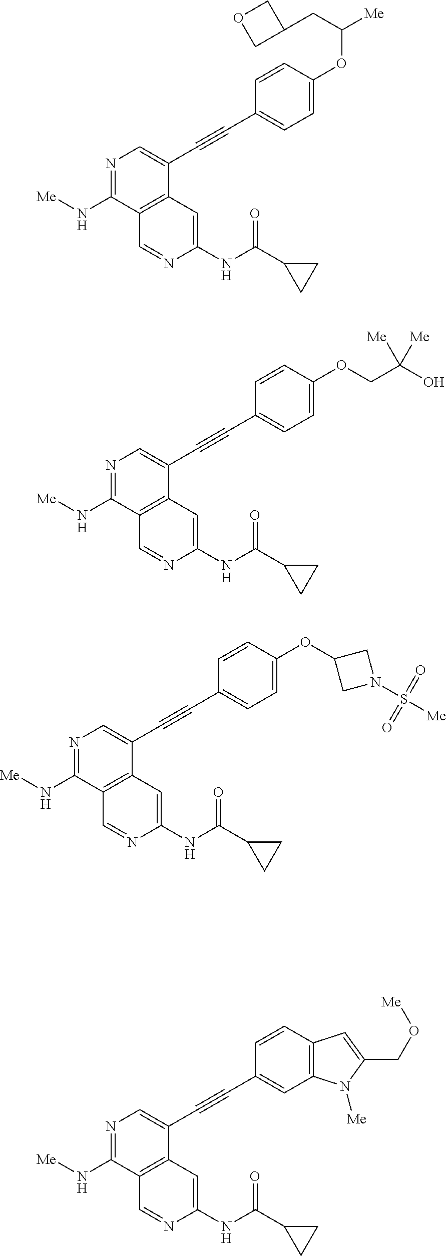 [SMILES: CNc1ncc(C#Cc2ccc(OC(C)CC3COC3)cc2)c2cc(NC(=O)C3CC3)ncc12 ; CNc1ncc(C#Cc2ccc(OCC(C)(C)O)cc2)c2cc(NC(=O)C3CC3)ncc12 ; CNc1ncc(C#Cc2ccc(OC3CN(S(C)(=O)=O)C3)cc2)c2cc(NC(=O)C3CC3)ncc12 ; CNc1ncc(C#Cc2ccc3cc(COC)n(C)c3c2)c2cc(NC(=O)C3CC3)ncc12]

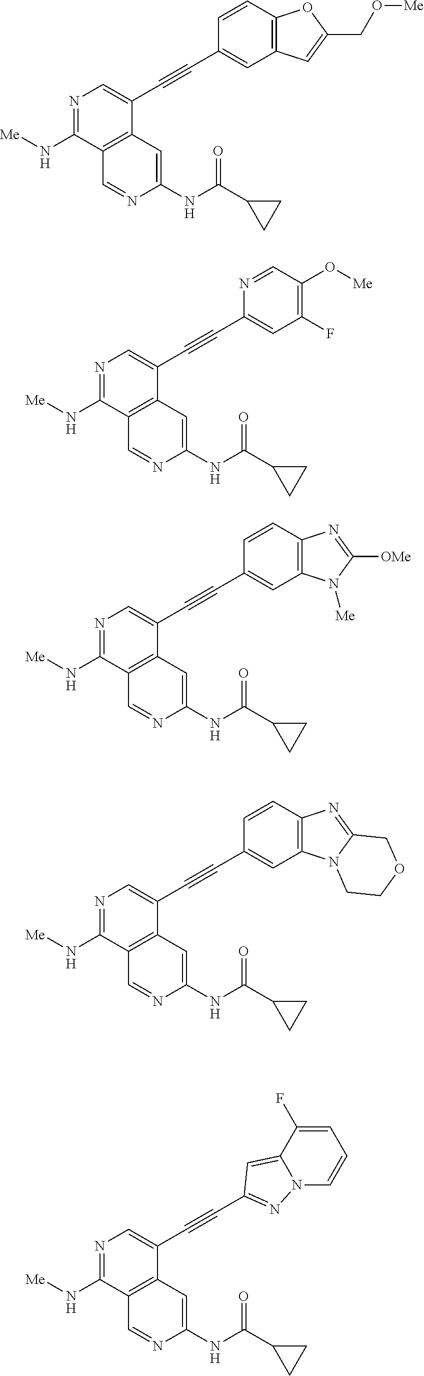

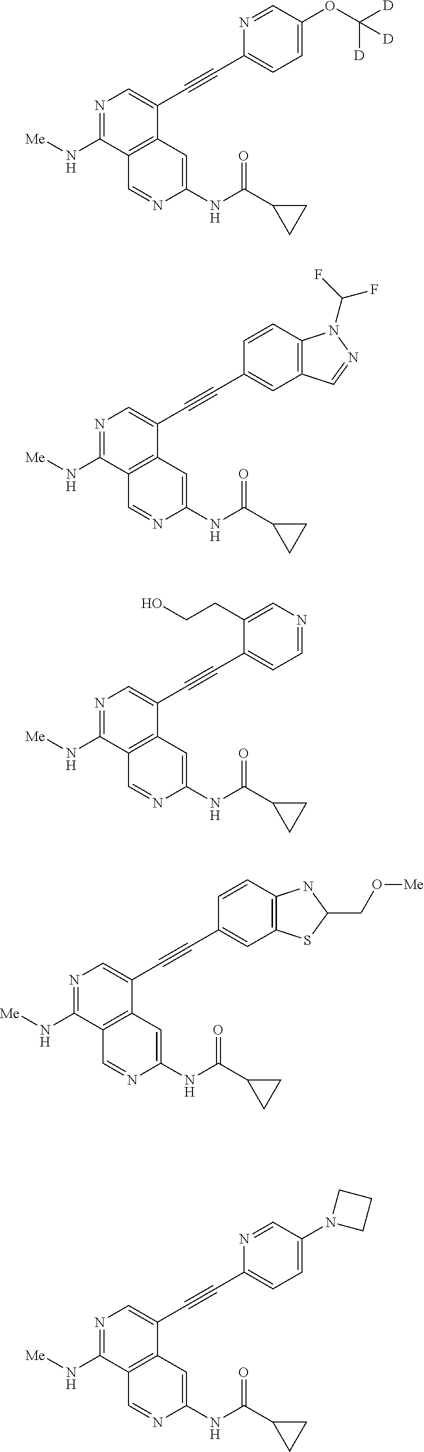 [SMILES: [2H]C([2H])([2H])Oc1ccc(C#Cc2cnc(NC)c3cnc(NC(=O)C4CC4)cc23)nc1 ; CNc1ncc(C#Cc2ccc3c(cnn3C(F)F)c2)c2cc(NC(=O)C3CC3)ncc12 ; CNc1ncc(C#Cc2ccncc2CCO)c2cc(NC(=O)C3CC3)ncc12 ; CNc1ncc(C#Cc2ccc3c(c2)SC(COC)N3)c2cc(NC(=O)C3CC3)ncc12 ; CNc1ncc(C#Cc2ccc(N3CCC3)cn2)c2cc(NC(=O)C3CC3)ncc12]

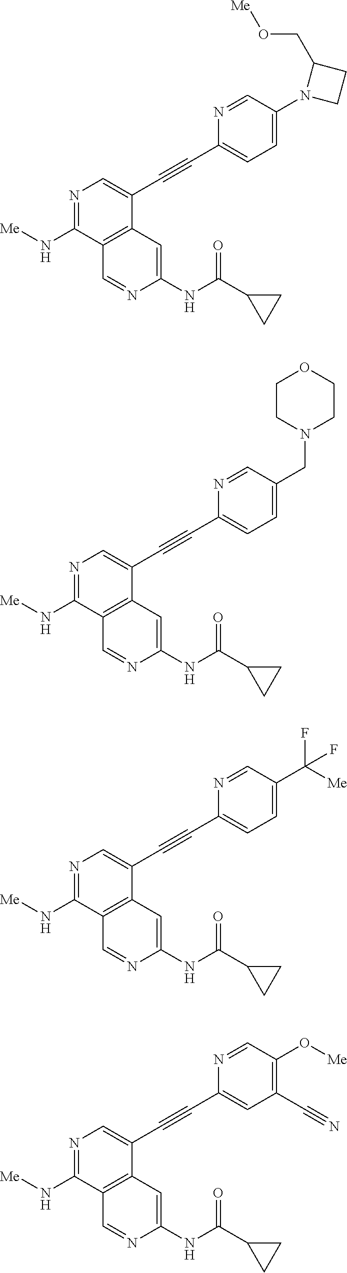 [SMILES: CNc1ncc(C#Cc2ccc(N3CCC3COC)cn2)c2cc(NC(=O)C3CC3)ncc12 ; CNc1ncc(C#Cc2ccc(CN3CCOCC3)cn2)c2cc(NC(=O)C3CC3)ncc12 ; CNc1ncc(C#Cc2ccc(C(C)(F)F)cn2)c2cc(NC(=O)C3CC3)ncc12 ; CNc1ncc(C#Cc2cc(C#N)c(OC)cn2)c2cc(NC(=O)C3CC3)ncc12]

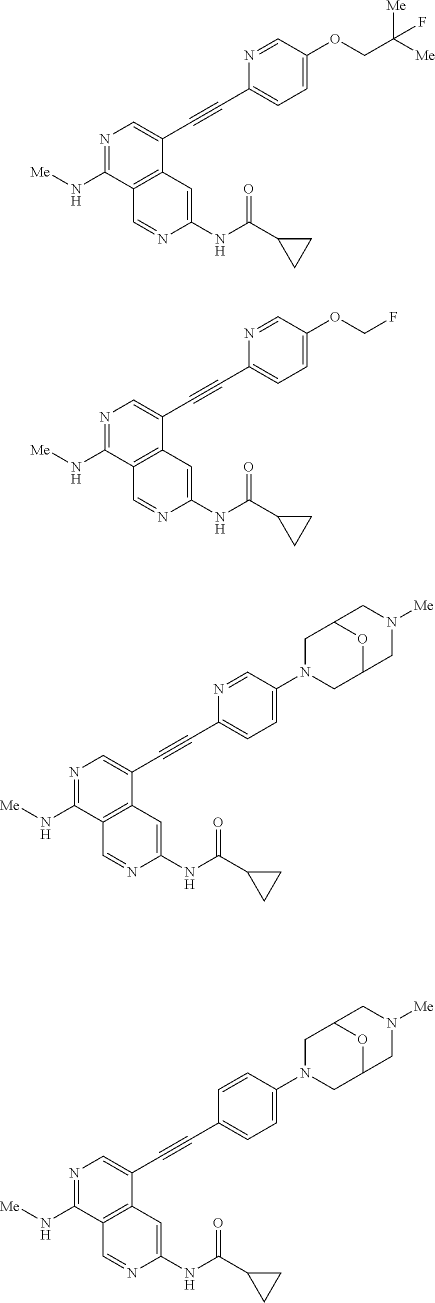 [SMILES: CNc1ncc(C#Cc2ccc(OCC(C)(C)F)cn2)c2cc(NC(=O)C3CC3)ncc12 ; CNc1ncc(C#Cc2ccc(OCF)cn2)c2cc(NC(=O)C3CC3)ncc12 ; CNc1ncc(C#Cc2ccc(N3CC4CN(C)CC(C3)O4)cn2)c2cc(NC(=O)C3CC3)ncc12 ; CNc1ncc(C#Cc2ccc(N3CC4CN(C)CC(C3)O4)cc2)c2cc(NC(=O)C3CC3)ncc12]

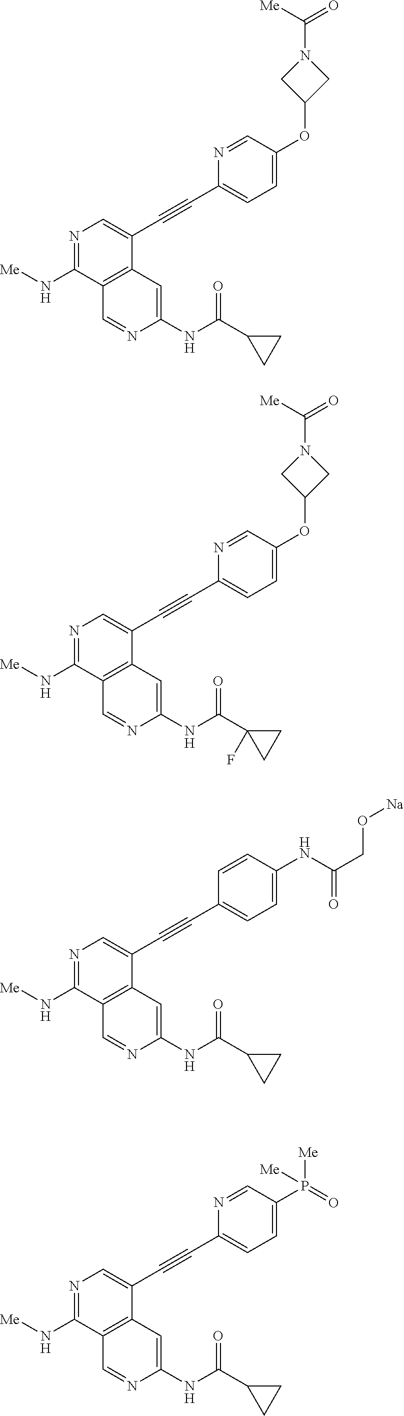

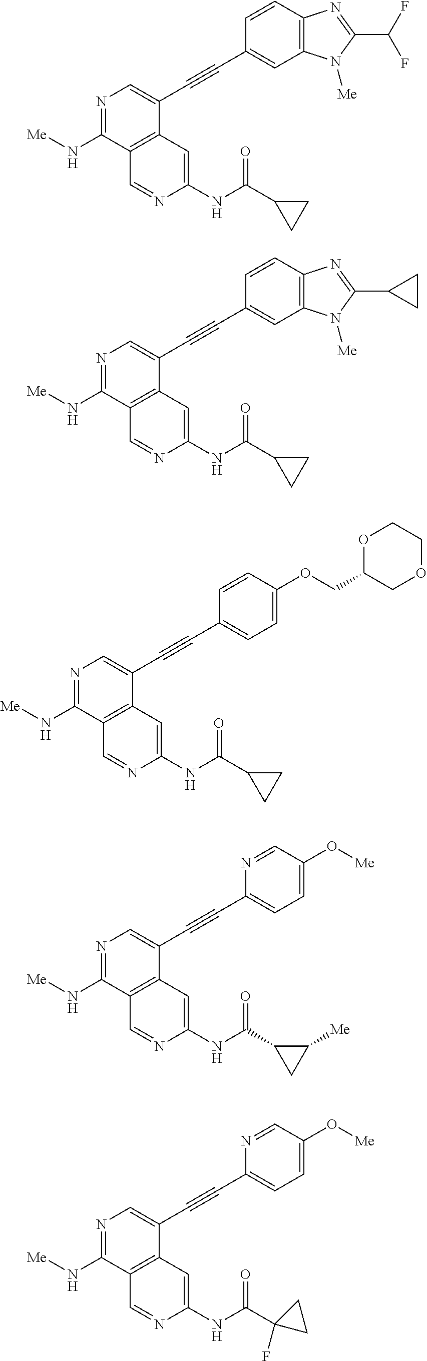

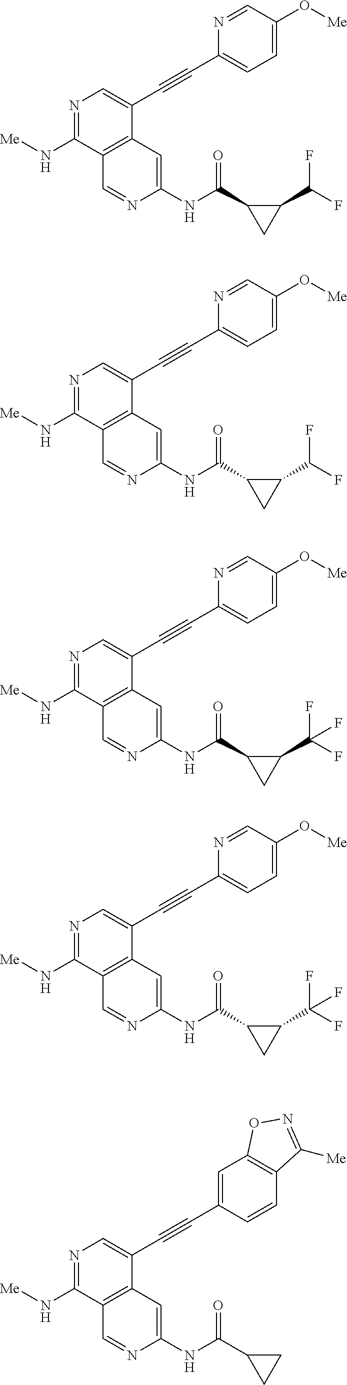

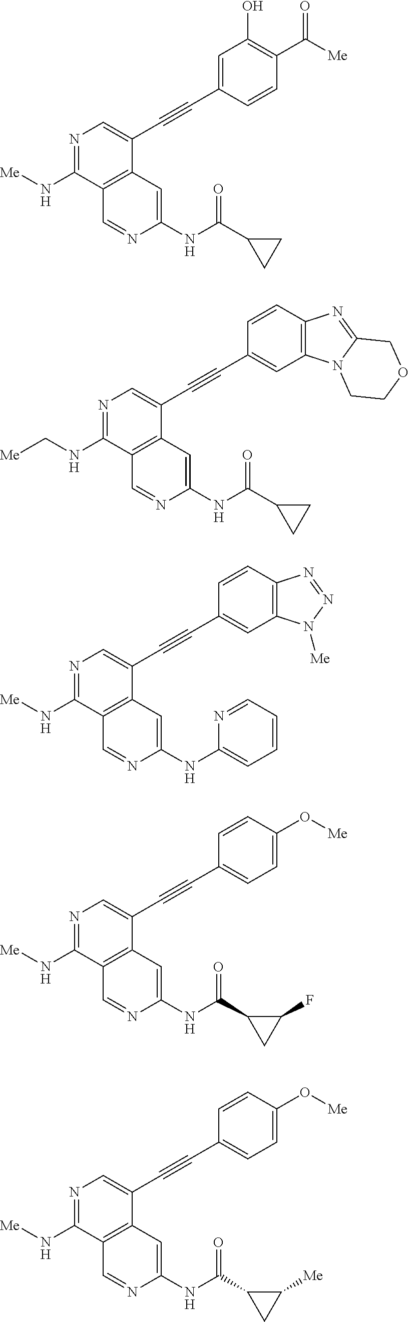

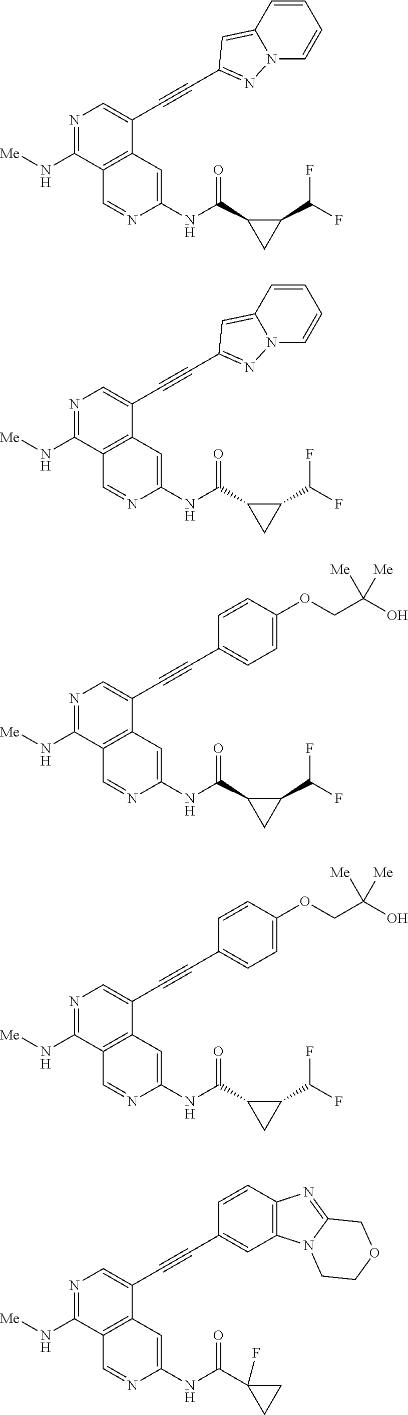

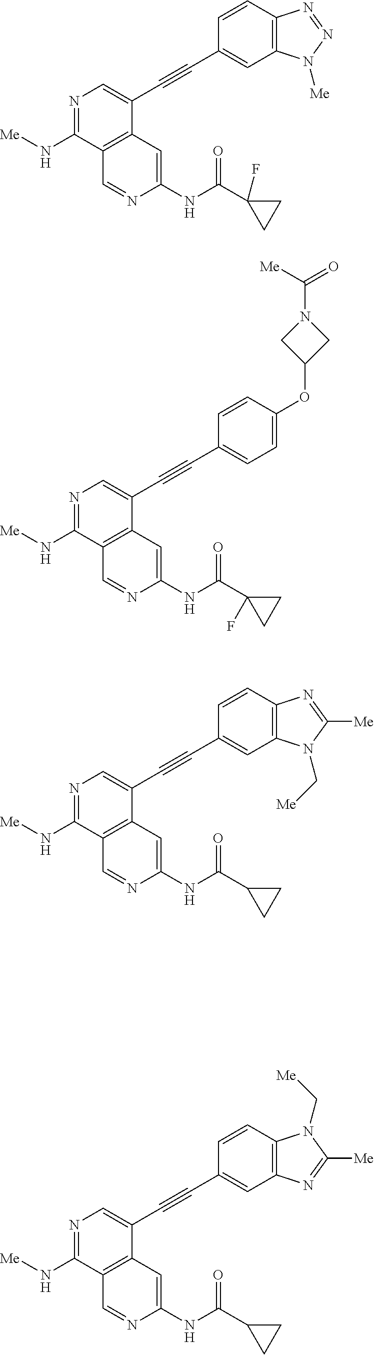

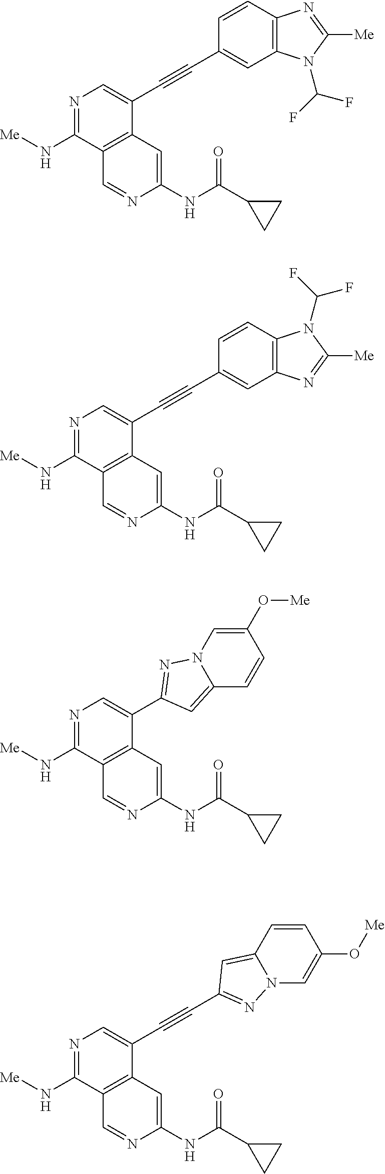 [SMILES: CNc1ncc(C#Cc2ccc3nc(C)n(C(F)F)c3c2)c2cc(NC(=O)C3CC3)ncc12 ; CNc1ncc(C#Cc2ccc3c(c2)nc(C)n3C(F)F)c2cc(NC(=O)C3CC3)ncc12 ; CNc1ncc(-c2cc3ccc(OC)cn3n2)c2cc(NC(=O)C3CC3)ncc12 ; CNc1ncc(C#Cc2cc3ccc(OC)cn3n2)c2cc(NC(=O)C3CC3)ncc12]

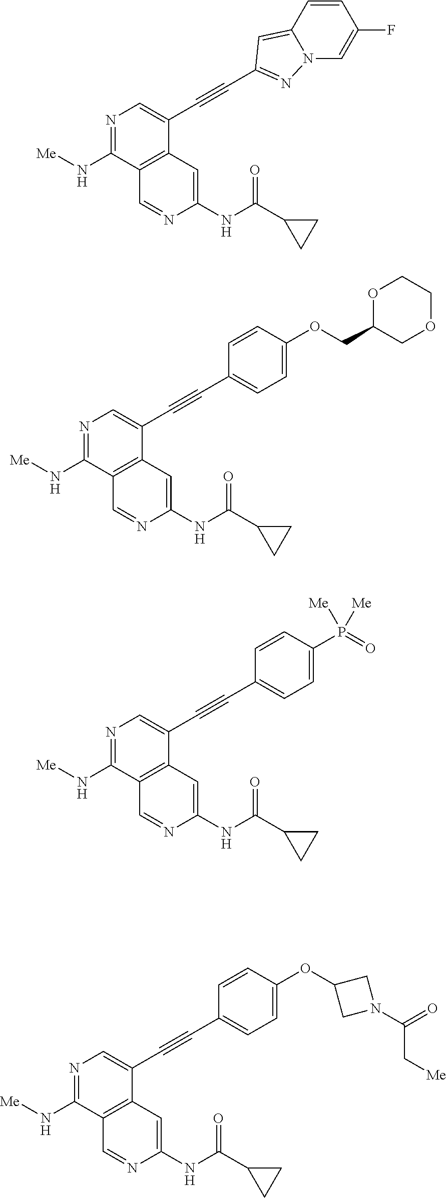 [SMILES: CNc1ncc(C#Cc2cc3ccc(F)cn3n2)c2cc(NC(=O)C3CC3)ncc12 ; CNc1ncc(C#Cc2ccc(OC[C@@H]3COCCO3)cc2)c2cc(NC(=O)C3CC3)ncc12 ; CNc1ncc(C#Cc2ccc(P(C)(C)=O)cc2)c2cc(NC(=O)C3CC3)ncc12 ; CCC(=O)N1CC(Oc2ccc(C#Cc3cnc(NC)c4cnc(NC(=O)C5CC5)cc34)cc2)C1]

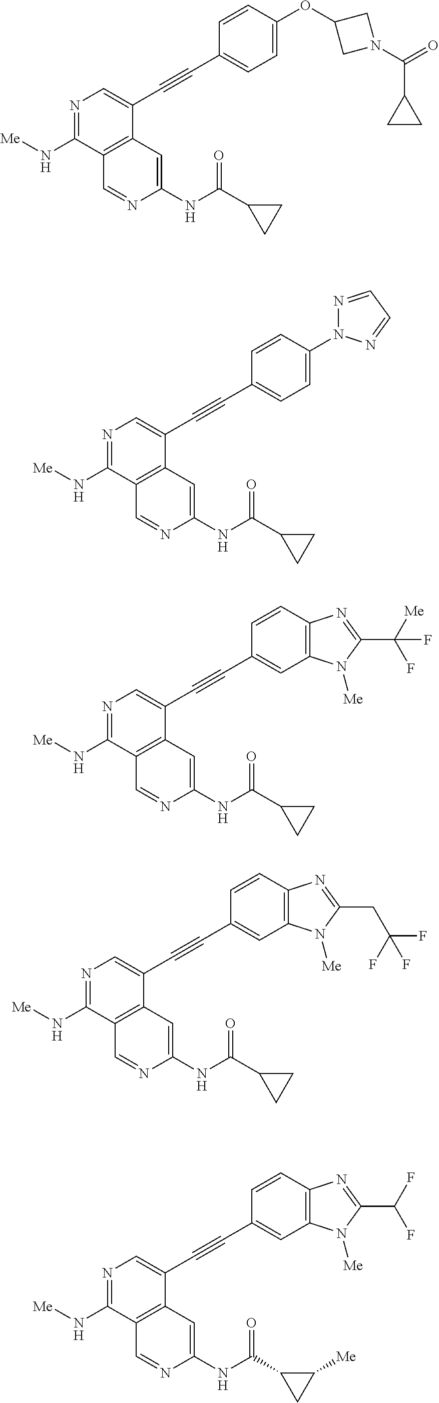

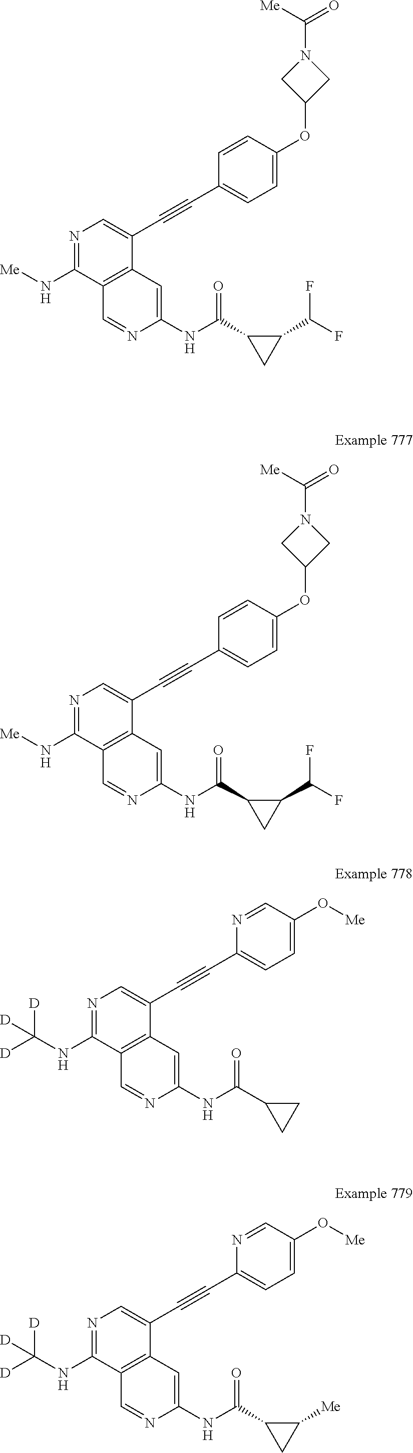 [SMILES: CNc1ncc(C#Cc2ccc(OC3CN(C(C)=O)C3)cc2)c2cc(NC(=O)[C@H]3C[C@H]3C(F)F)ncc12 ; CNc1ncc(C#Cc2ccc(OC3CN(C(C)=O)C3)cc2)c2cc(NC(=O)[C@@H]3C[C@@H]3C(F)F)ncc12 ; [2H]C([2H])([2H])Nc1ncc(C#Cc2ccc(OC)cn2)c2cc(NC(=O)C3CC3)ncc12 ; [2H]C([2H])([2H])Nc1ncc(C#Cc2ccc(OC)cn2)c2cc(NC(=O)[C@H]3C[C@H]3C)ncc12]

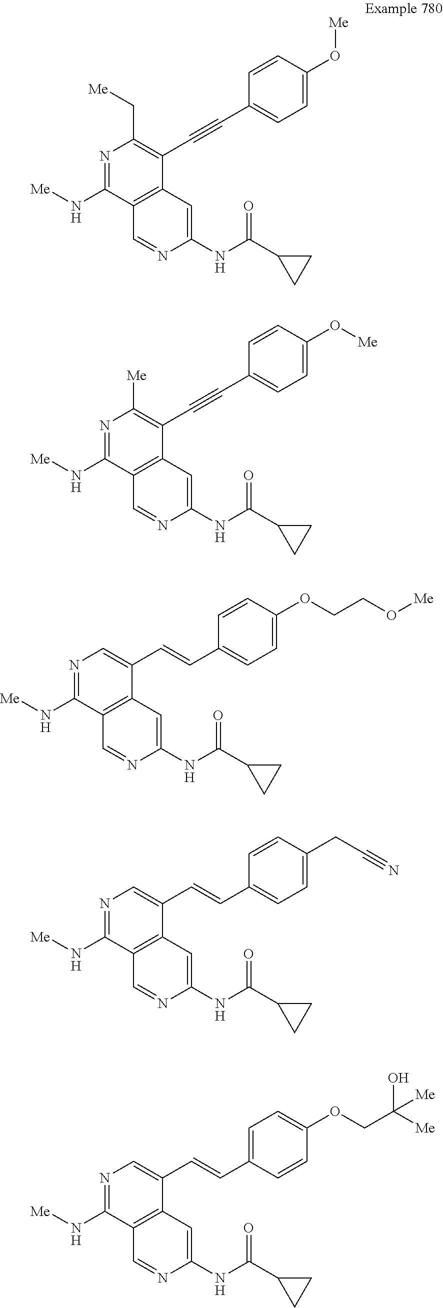

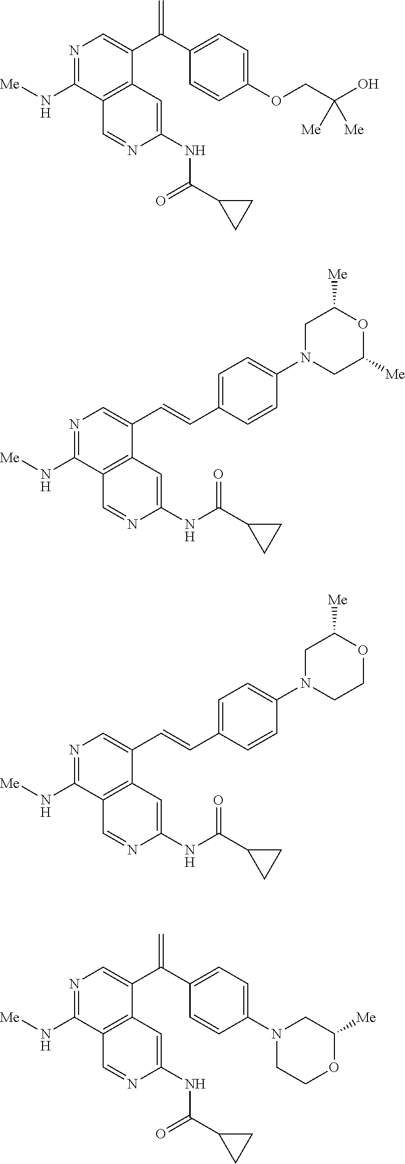 [SMILES: C=C(c1ccc(OCC(C)(C)O)cc1)c1cnc(NC)c2cnc(NC(=O)C3CC3)cc12 ; CNc1ncc(/C=C/c2ccc(N3C[C@@H](C)O[C@@H](C)C3)cc2)c2cc(NC(=O)C3CC3)ncc12 ; CNc1ncc(/C=C/c2ccc(N3CCO[C@@H](C)C3)cc2)c2cc(NC(=O)C3CC3)ncc12 ; C=C(c1ccc(N2CCO[C@@H](C)C2)cc1)c1cnc(NC)c2cnc(NC(=O)C3CC3)cc12]

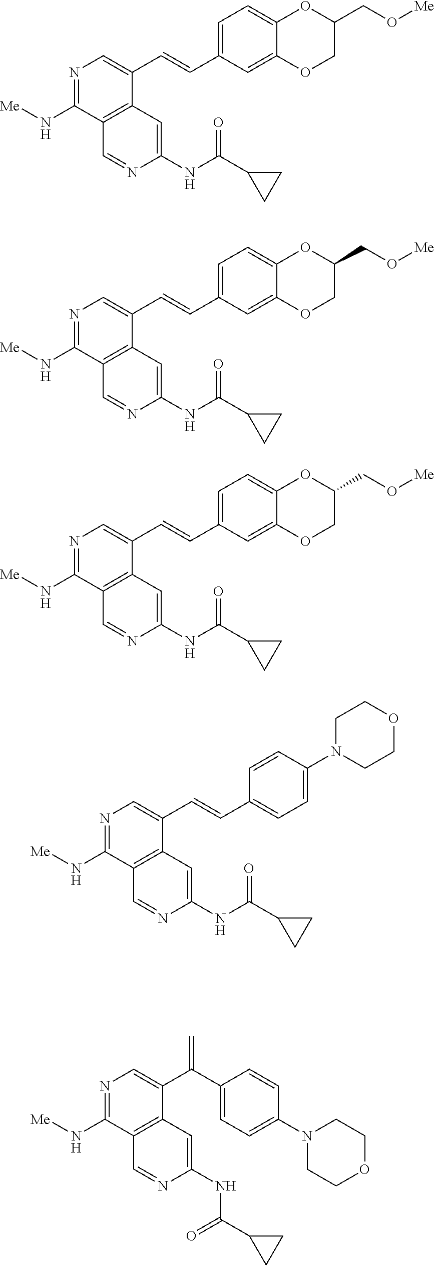

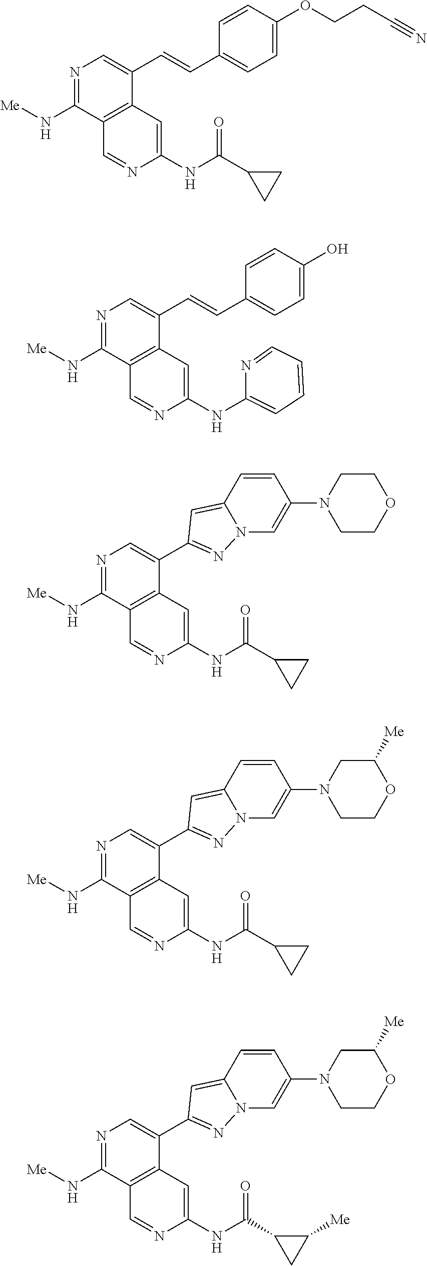 [SMILES: CNc1ncc(/C=C/c2ccc(OCCC#N)cc2)c2cc(NC(=O)C3CC3)ncc12 ; CNc1ncc(/C=C/c2ccc(O)cc2)c2cc(Nc3ccccn3)ncc12 ; CNc1ncc(-c2cc3ccc(N4CCOCC4)cn3n2)c2cc(NC(=O)C3CC3)ncc12 ; CNc1ncc(-c2cc3ccc(N4CCO[C@@H](C)C4)cn3n2)c2cc(NC(=O)C3CC3)ncc12 ; CNc1ncc(-c2cc3ccc(N4CCO[C@@H](C)C4)cn3n2)c2cc(NC(=O)[C@H]3C[C@H]3C)ncc12]

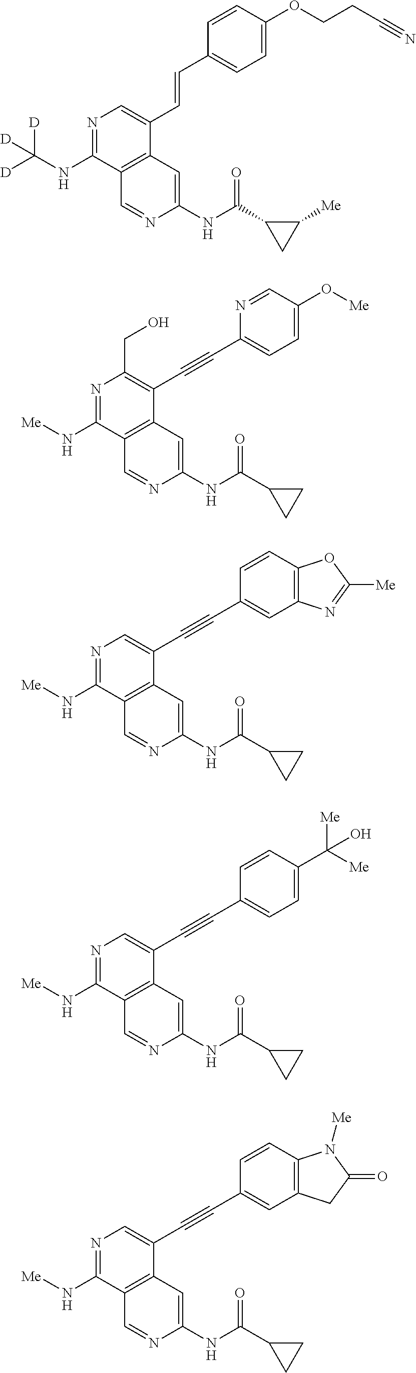

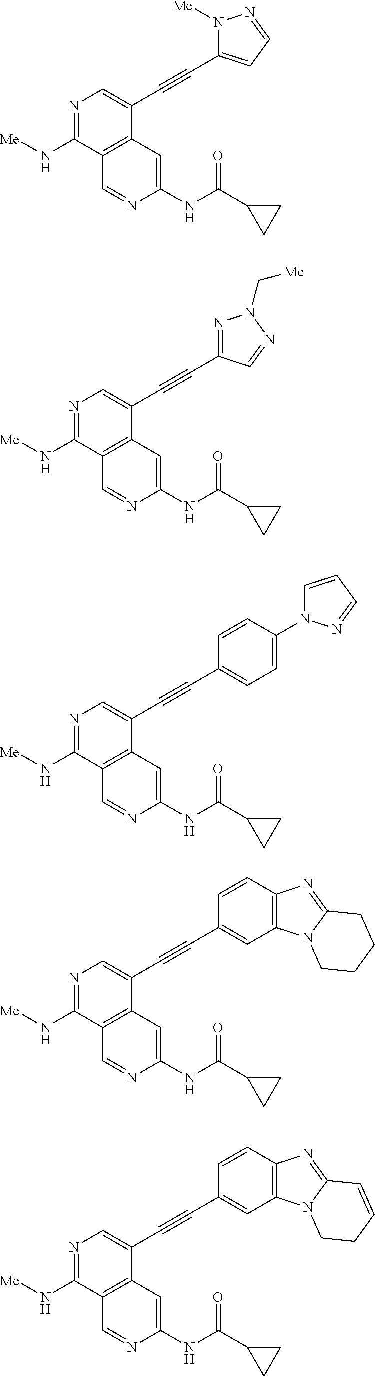

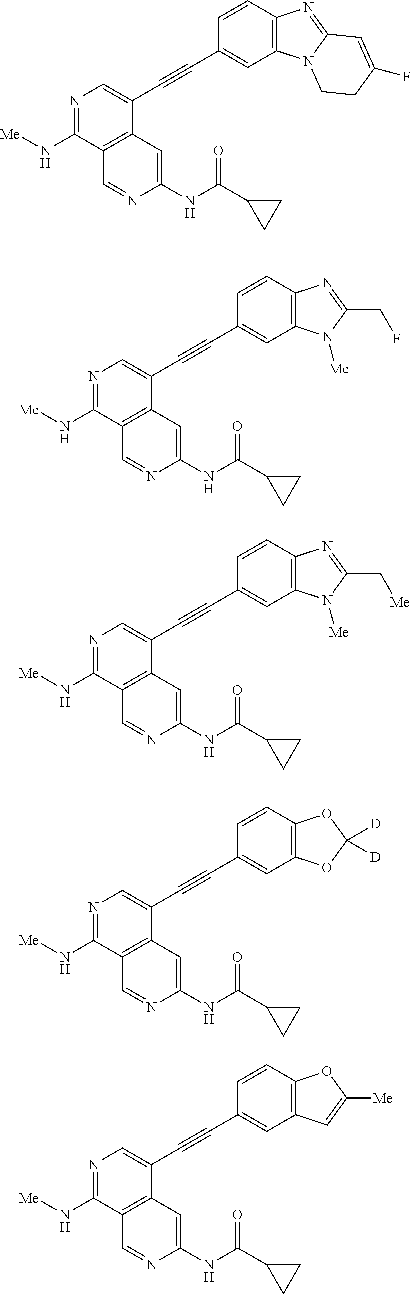

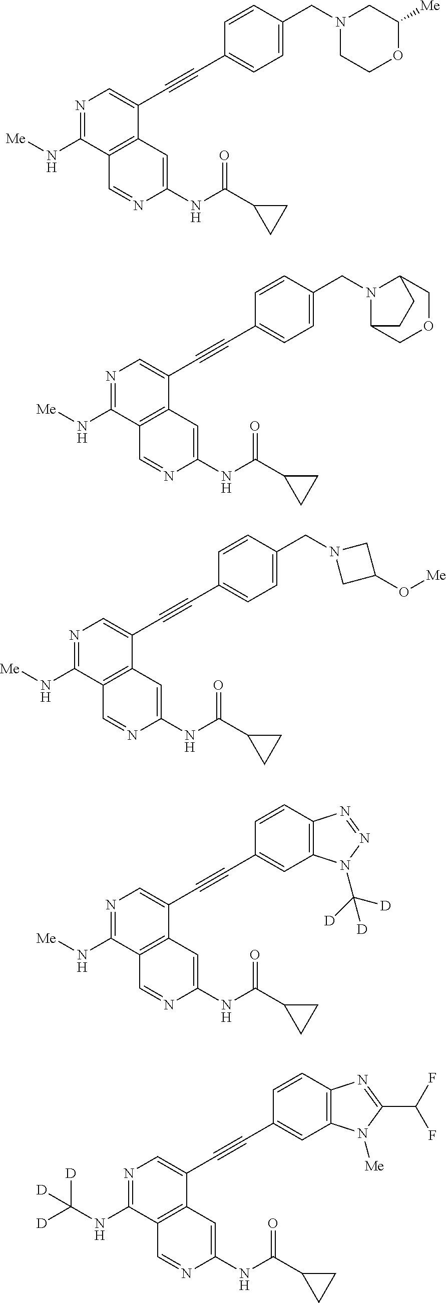

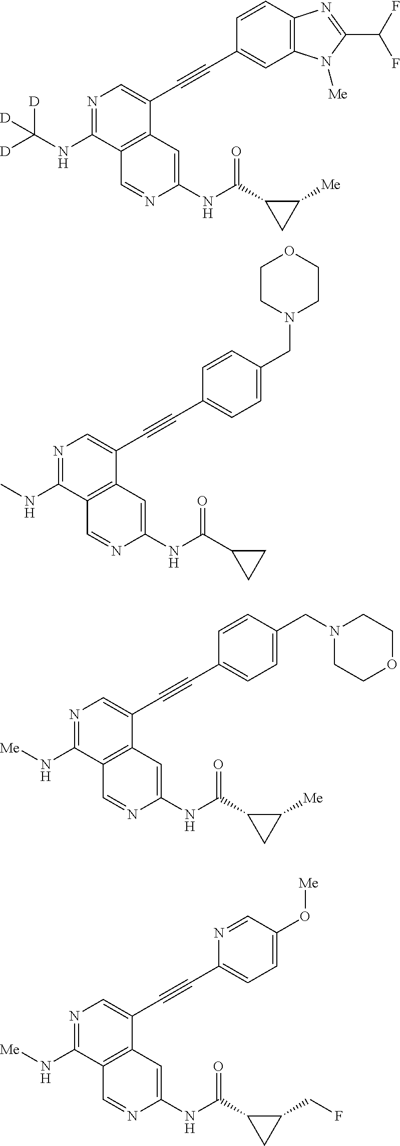

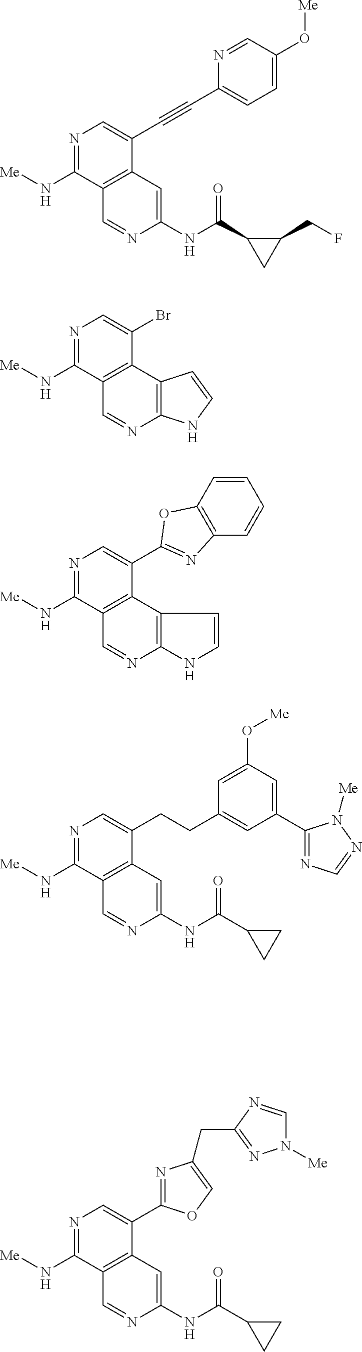 [SMILES: CNc1ncc(C#Cc2ccc(OC)cn2)c2cc(NC(=O)[C@@H]3C[C@@H]3CF)ncc12 ; CNc1ncc(Br)c2c1cnc1[nH]ccc12 ; CNc1ncc(-c2nc3ccccc3o2)c2c1cnc1[nH]ccc12 ; CNc1ncc(CCc2cc(OC)cc(-c3ncnn3C)c2)c2cc(NC(=O)C3CC3)ncc12 ; CNc1ncc(-c2nc(Cc3ncn(C)n3)co2)c2cc(NC(=O)C3CC3)ncc12]

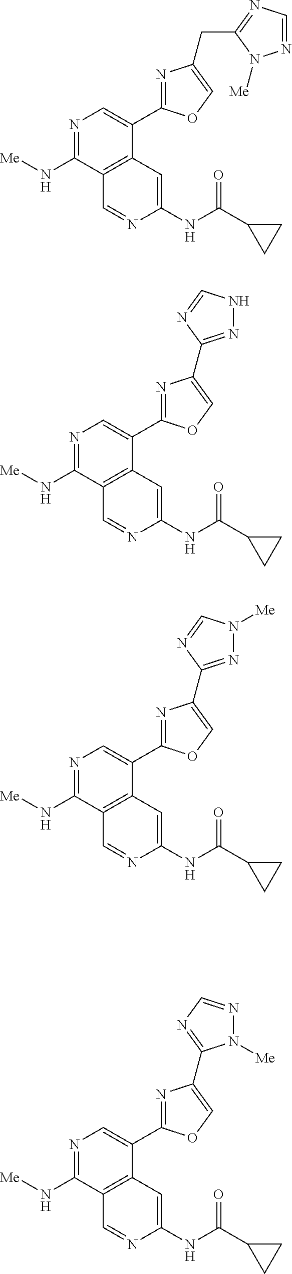

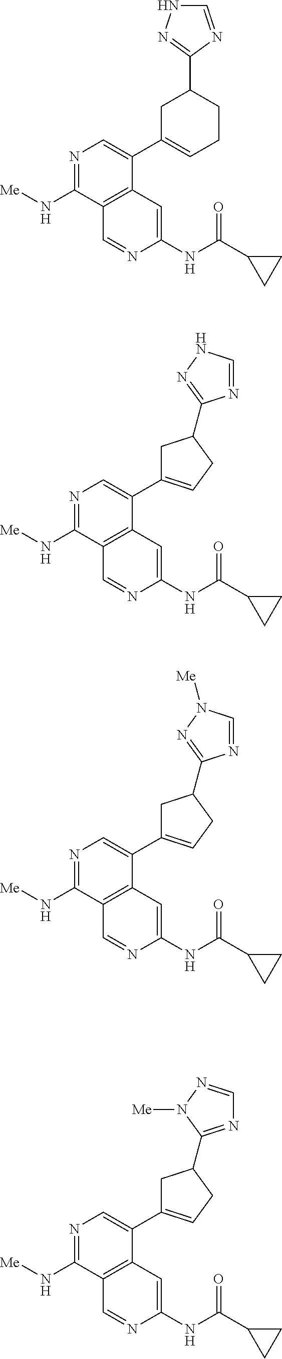 [SMILES: CNc1ncc(C2=CCCC(c3nc[nH]n3)C2)c2cc(NC(=O)C3CC3)ncc12 ; CNc1ncc(C2=CCC(c3nc[nH]n3)C2)c2cc(NC(=O)C3CC3)ncc12 ; CNc1ncc(C2=CCC(c3ncn(C)n3)C2)c2cc(NC(=O)C3CC3)ncc12 ; CNc1ncc(C2=CCC(c3ncnn3C)C2)c2cc(NC(=O)C3CC3)ncc12]

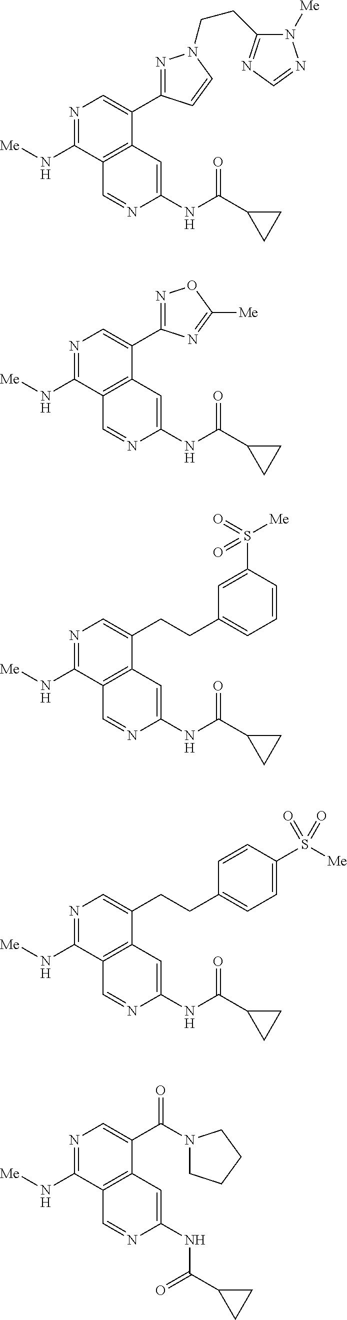

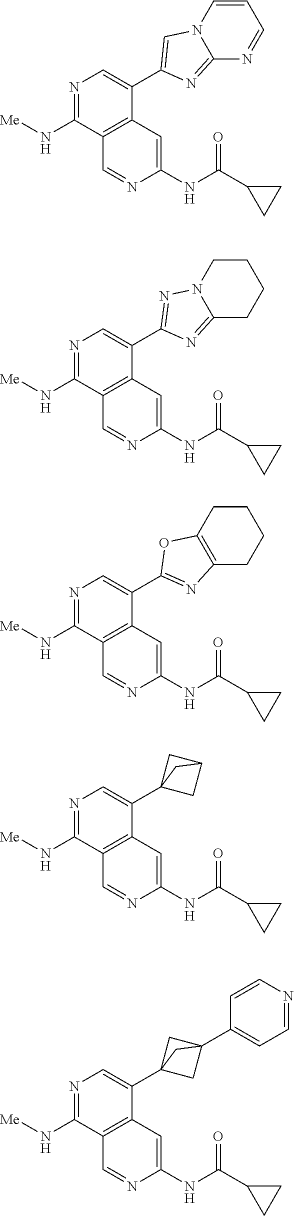

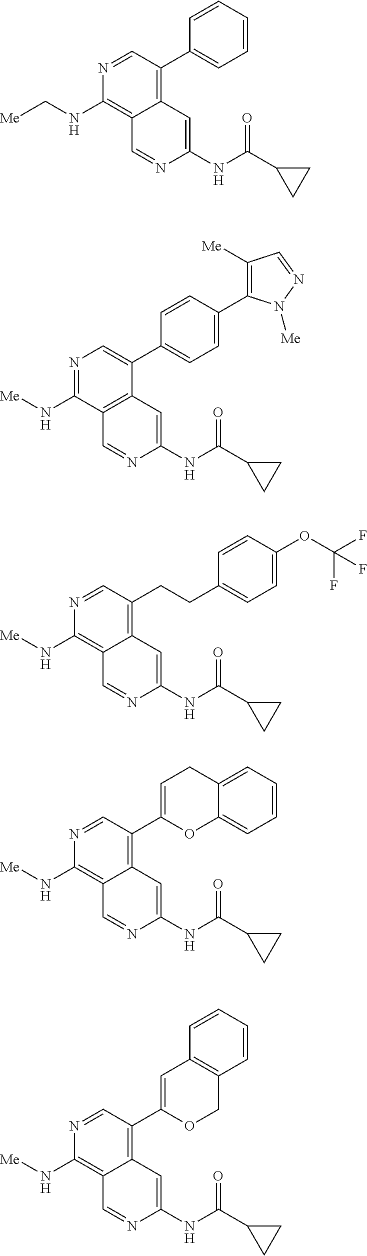

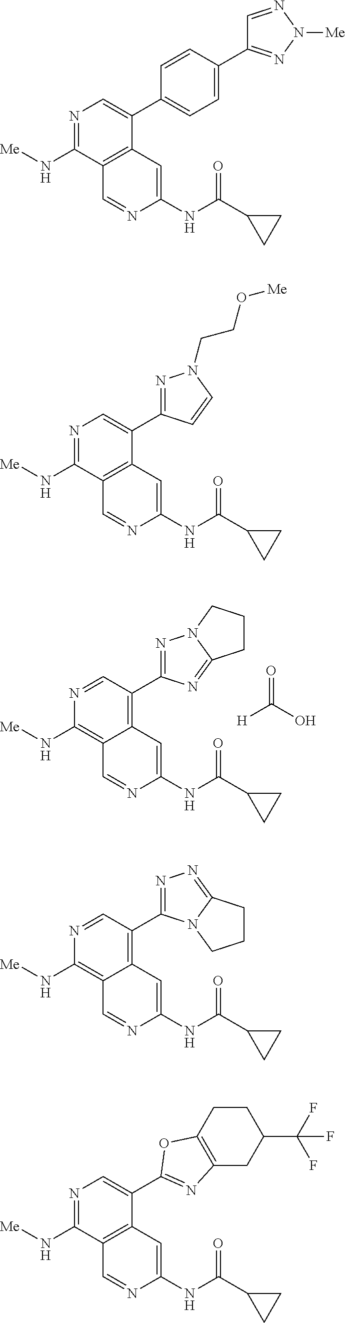

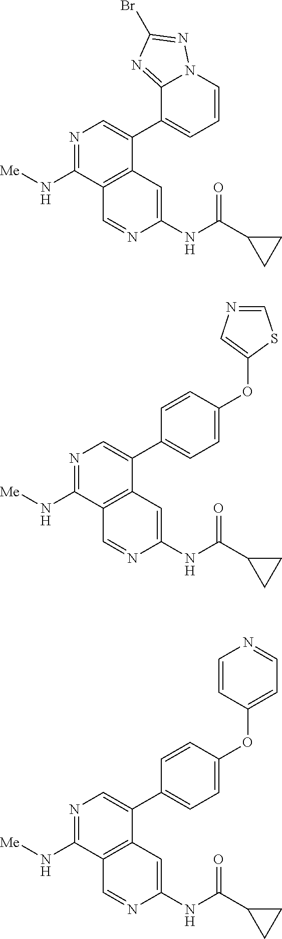

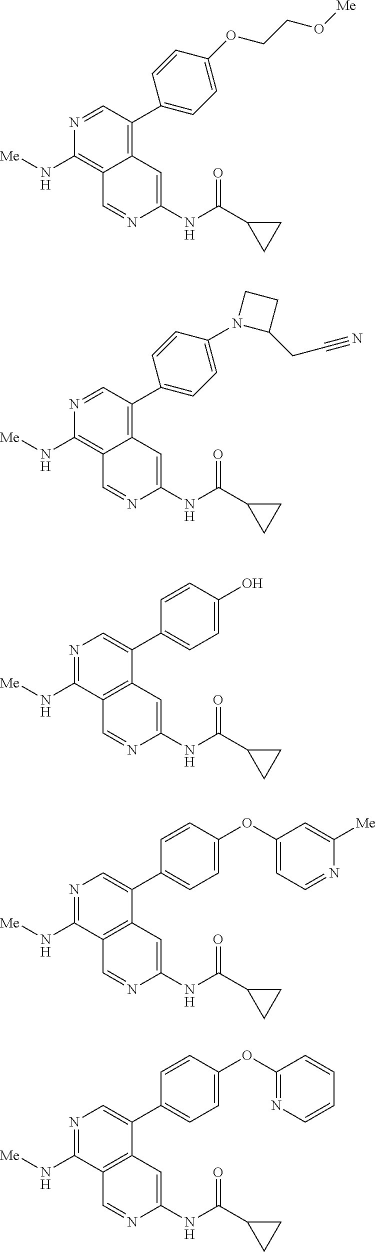

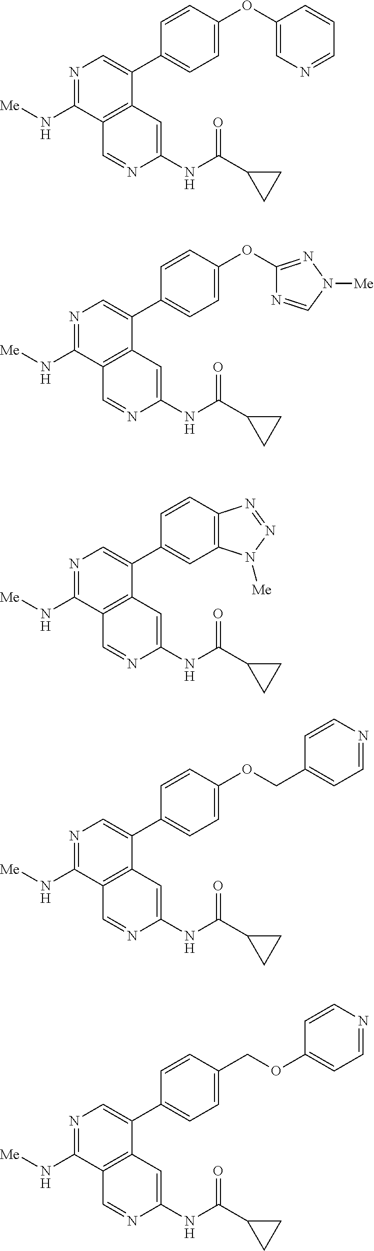 [SMILES: CNc1ncc(-c2ccc(Oc3cccnc3)cc2)c2cc(NC(=O)C3CC3)ncc12 ; CNc1ncc(-c2ccc(Oc3ncn(C)n3)cc2)c2cc(NC(=O)C3CC3)ncc12 ; CNc1ncc(-c2ccc3nnn(C)c3c2)c2cc(NC(=O)C3CC3)ncc12 ; CNc1ncc(-c2ccc(OCc3ccncc3)cc2)c2cc(NC(=O)C3CC3)ncc12 ; CNc1ncc(-c2ccc(COc3ccncc3)cc2)c2cc(NC(=O)C3CC3)ncc12]

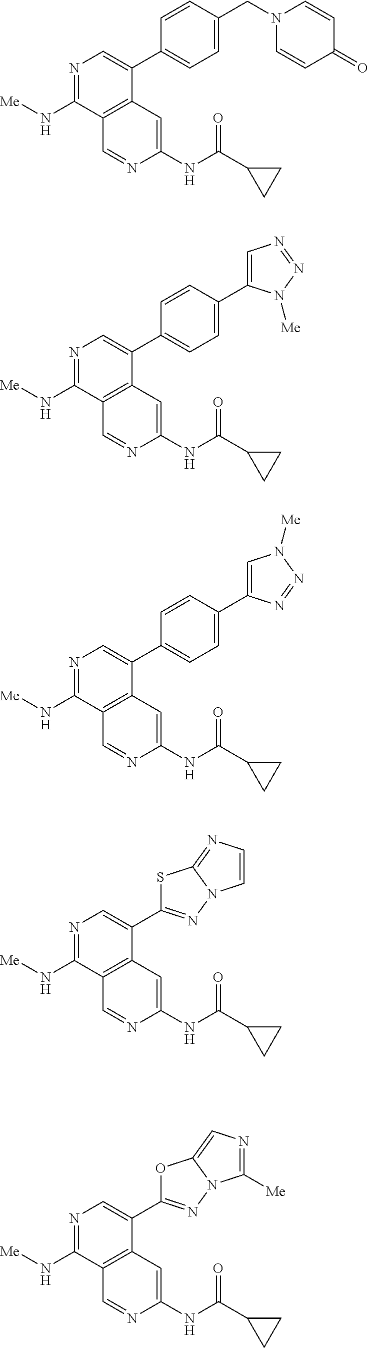

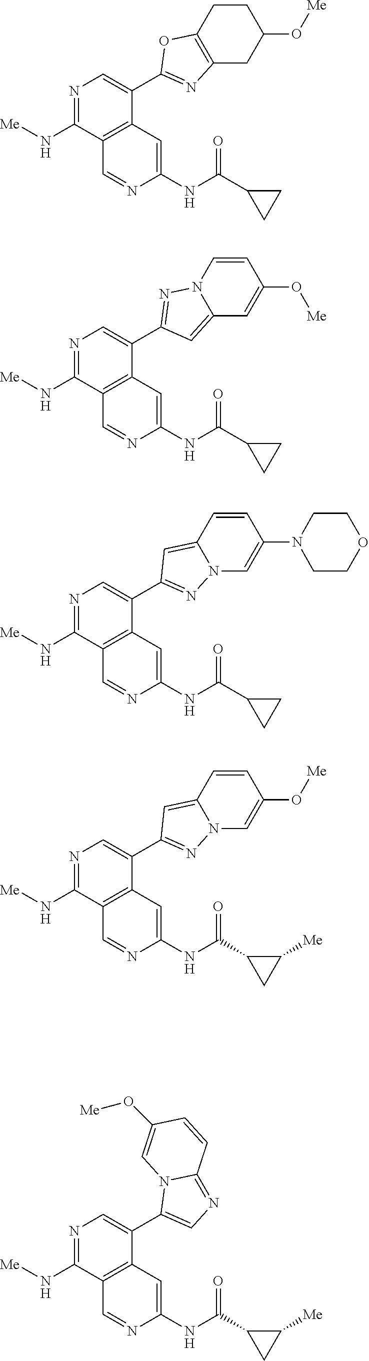

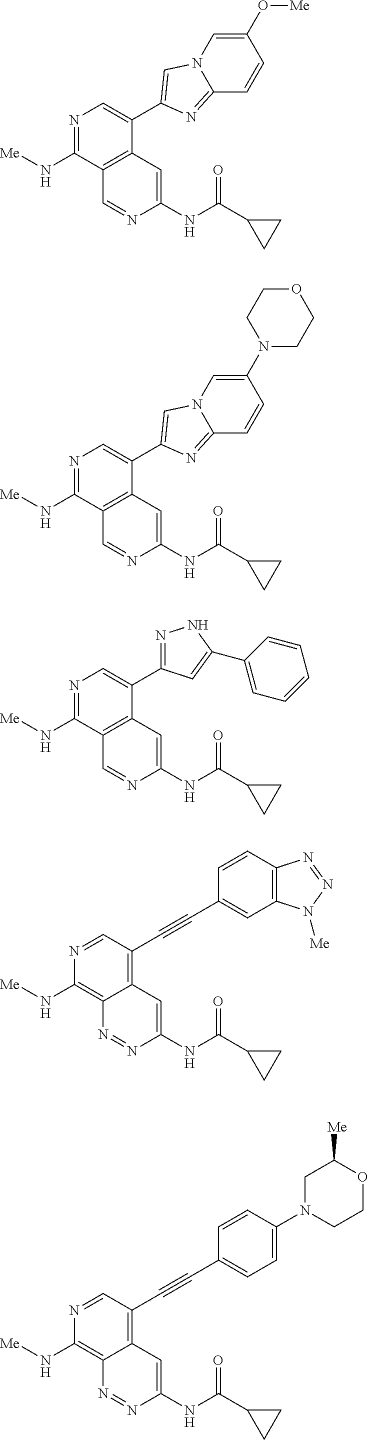

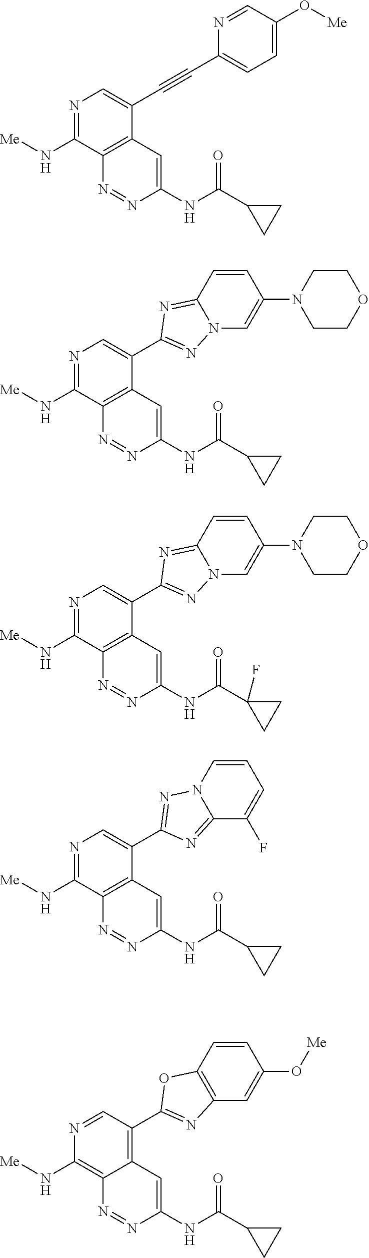

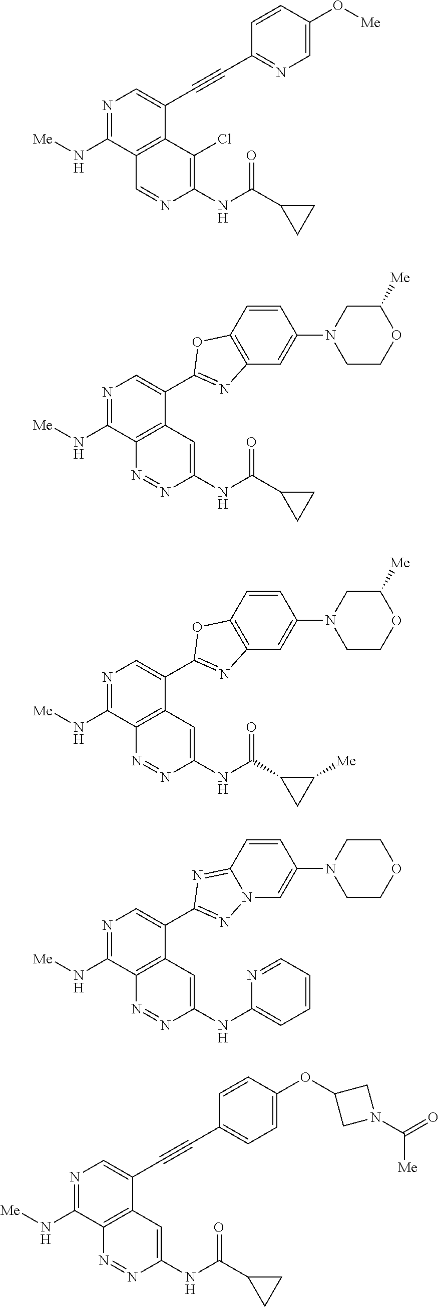

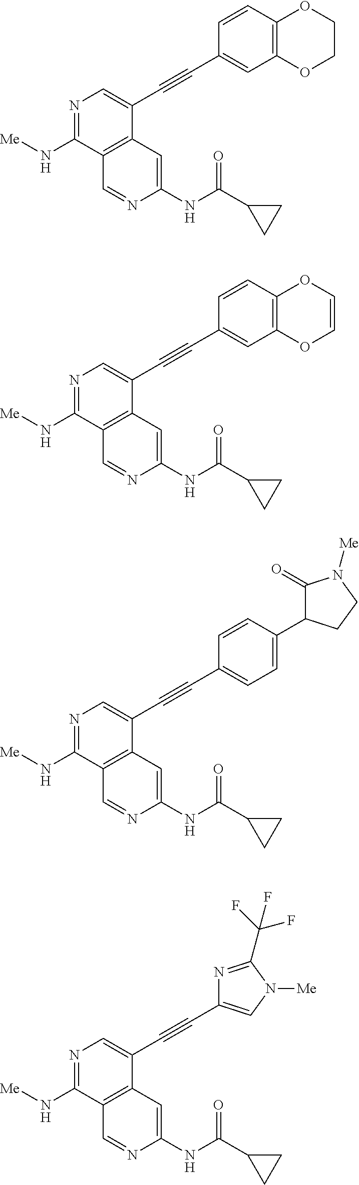 [SMILES: CNc1ncc(C#Cc2ccc3c(c2)OCCO3)c2cc(NC(=O)C3CC3)ncc12 ; CNc1ncc(C#Cc2ccc3c(c2)OC=CO3)c2cc(NC(=O)C3CC3)ncc12 ; CNc1ncc(C#Cc2ccc(C3CCN(C)C3=O)cc2)c2cc(NC(=O)C3CC3)ncc12 ; CNc1ncc(C#Cc2cn(C)c(C(F)(F)F)n2)c2cc(NC(=O)C3CC3)ncc12]

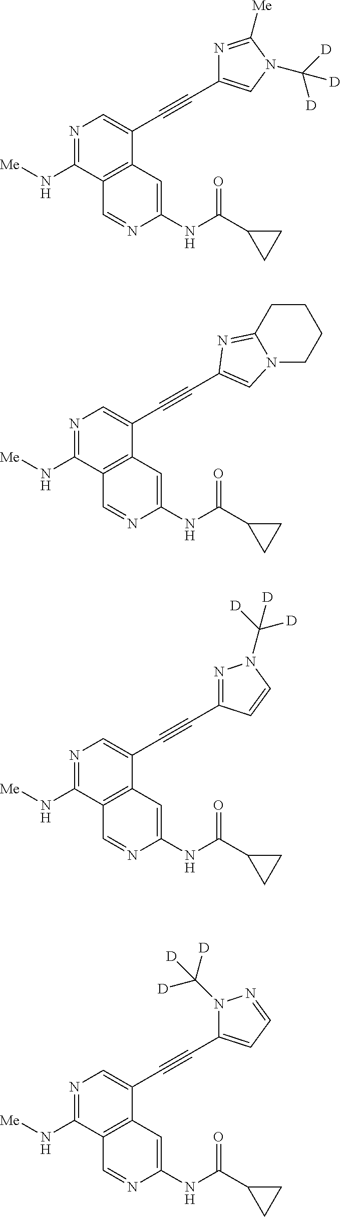

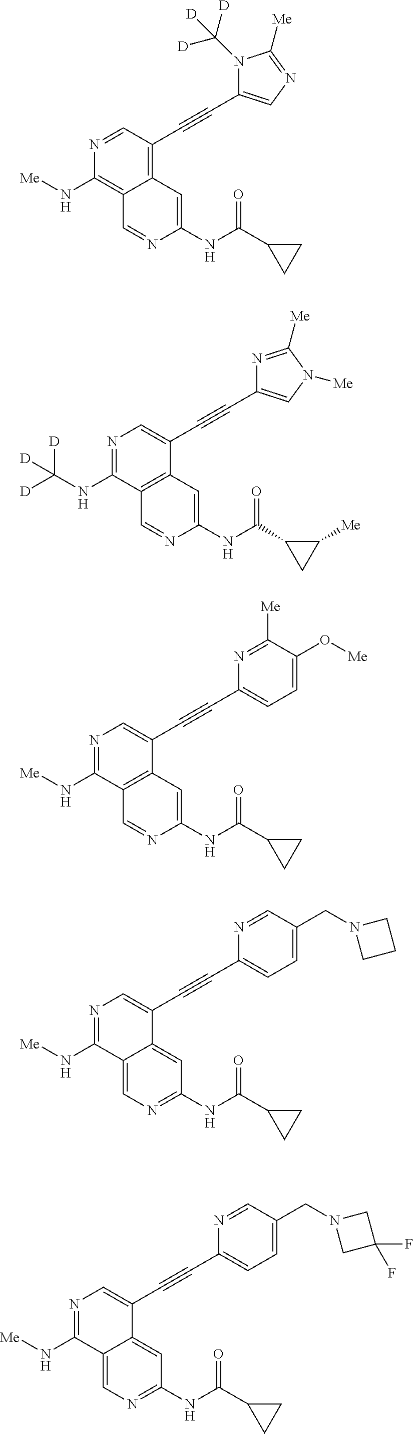

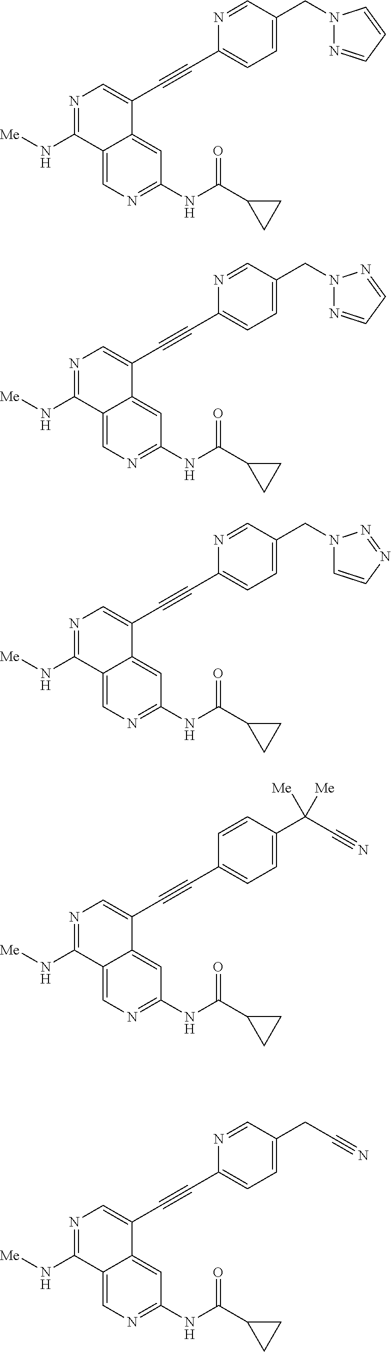

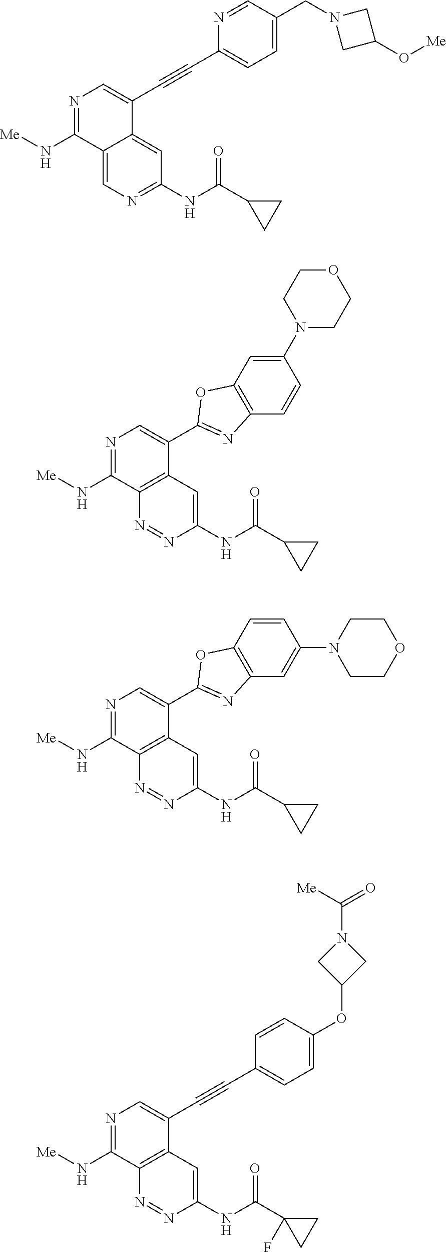 [SMILES: CNc1ncc(C#Cc2ccc(CN3CC(OC)C3)cn2)c2cc(NC(=O)C3CC3)ncc12 ; CNc1ncc(-c2nc3ccc(N4CCOCC4)cc3o2)c2cc(NC(=O)C3CC3)nnc12 ; CNc1ncc(-c2nc3cc(N4CCOCC4)ccc3o2)c2cc(NC(=O)C3CC3)nnc12 ; CNc1ncc(C#Cc2ccc(OC3CN(C(C)=O)C3)cc2)c2cc(NC(=O)C3(F)CC3)nnc12]

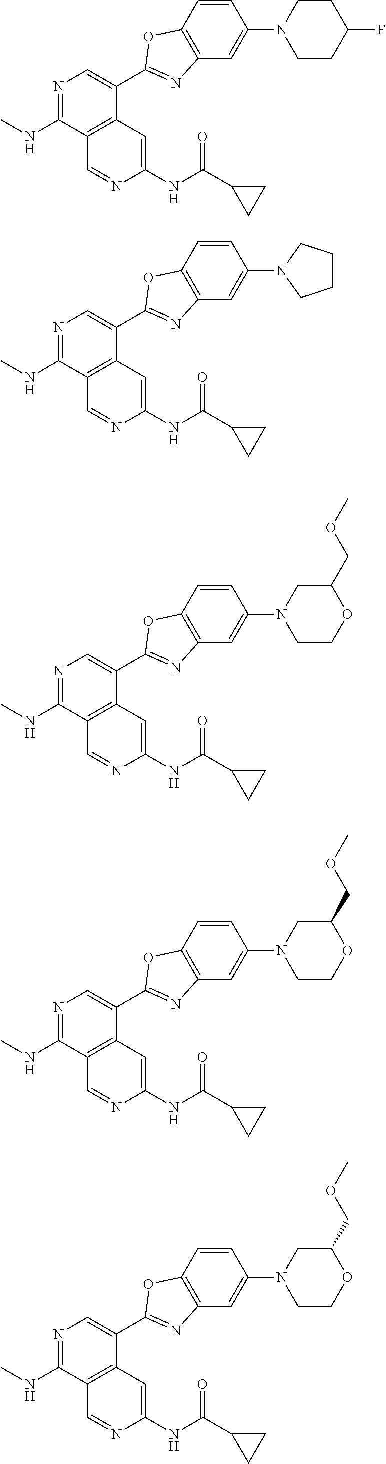

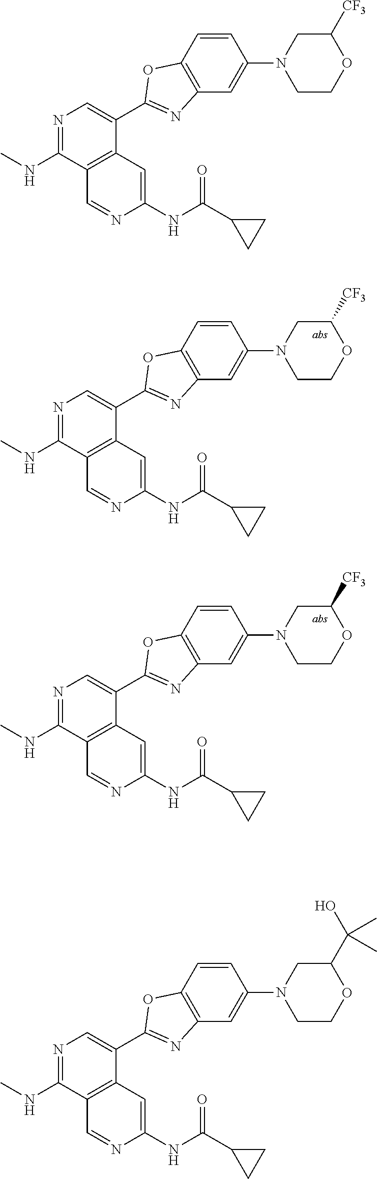 [SMILES: CNc1ncc(-c2nc3cc(N4CCOC(C(F)(F)F)C4)ccc3o2)c2cc(NC(=O)C3CC3)ncc12 ; CNc1ncc(-c2nc3cc(N4CCO[C@@H](C(F)(F)F)C4)ccc3o2)c2cc(NC(=O)C3CC3)ncc12 ; CNc1ncc(-c2nc3cc(N4CCO[C@H](C(F)(F)F)C4)ccc3o2)c2cc(NC(=O)C3CC3)ncc12 ; CNc1ncc(-c2nc3cc(N4CCOC(C(C)(C)O)C4)ccc3o2)c2cc(NC(=O)C3CC3)ncc12]

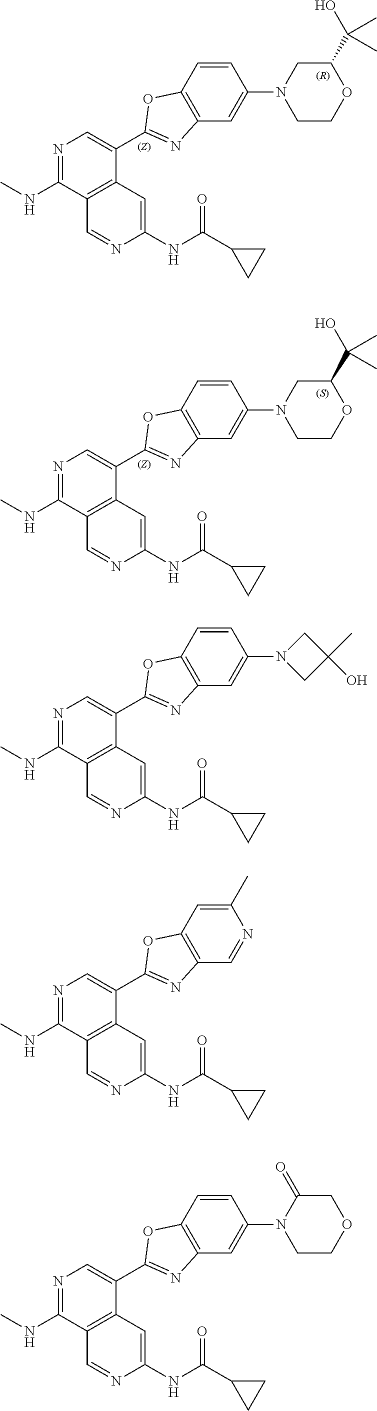

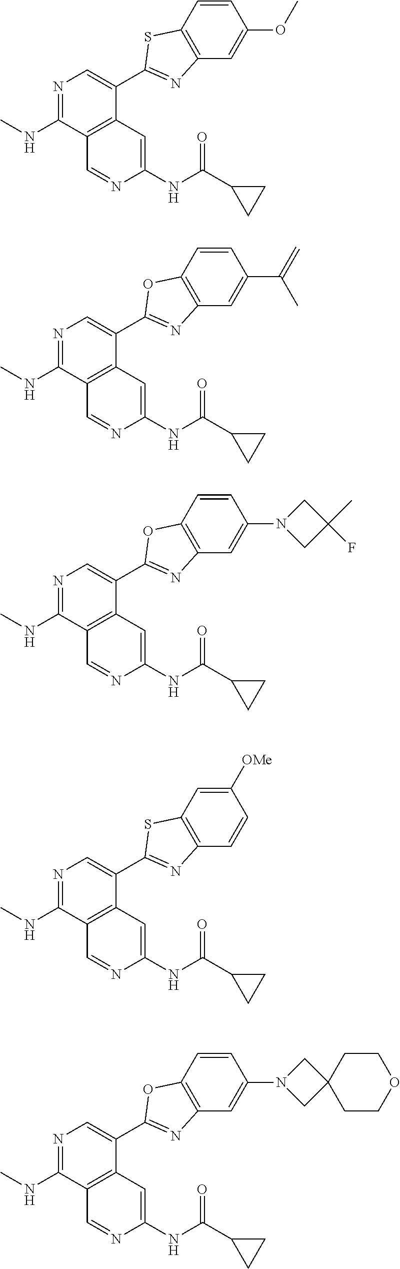

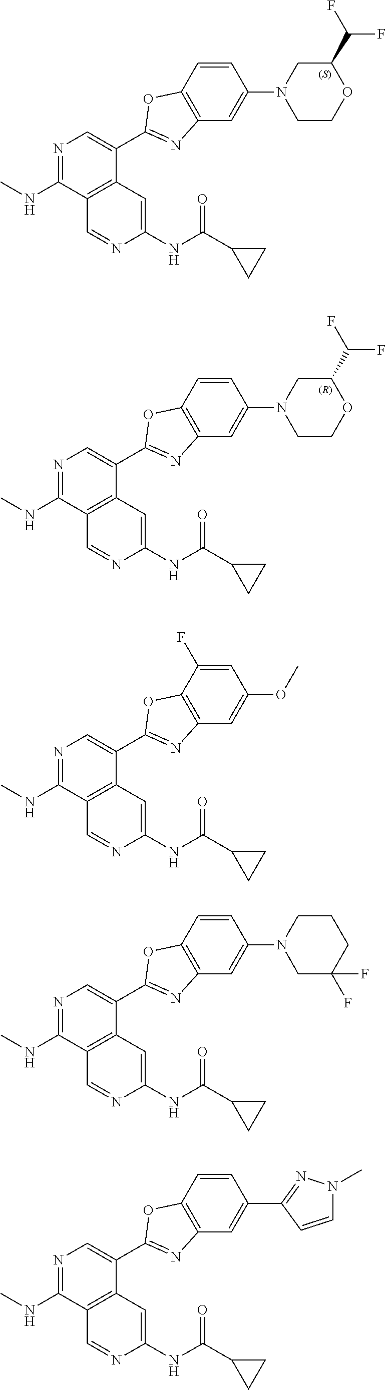

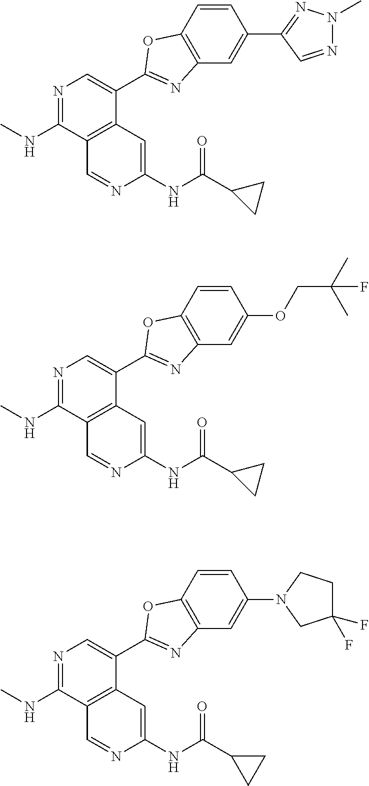

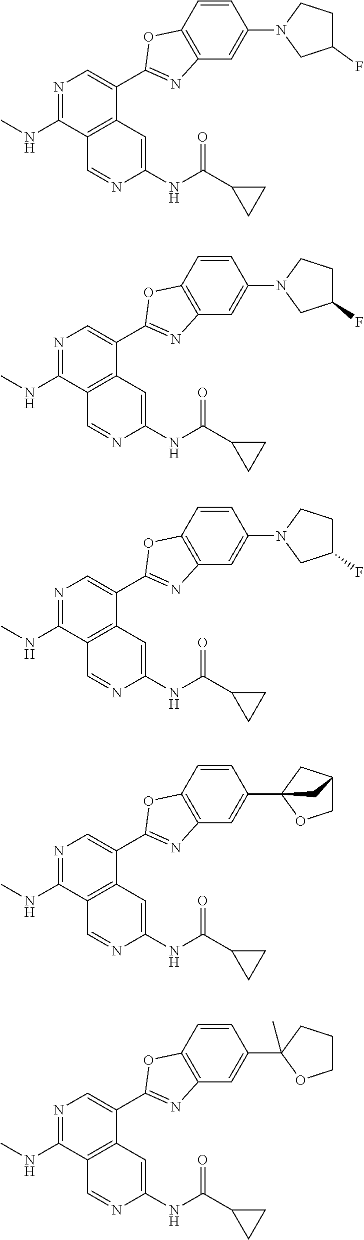

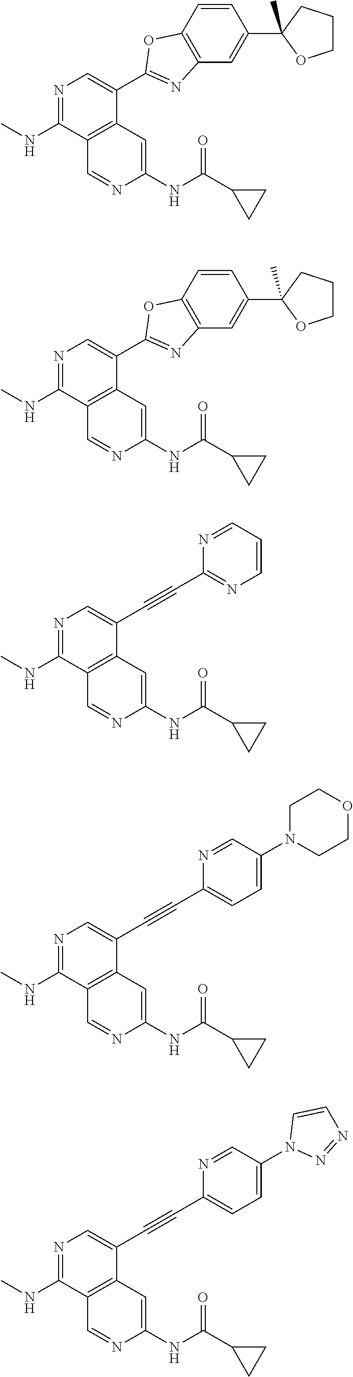

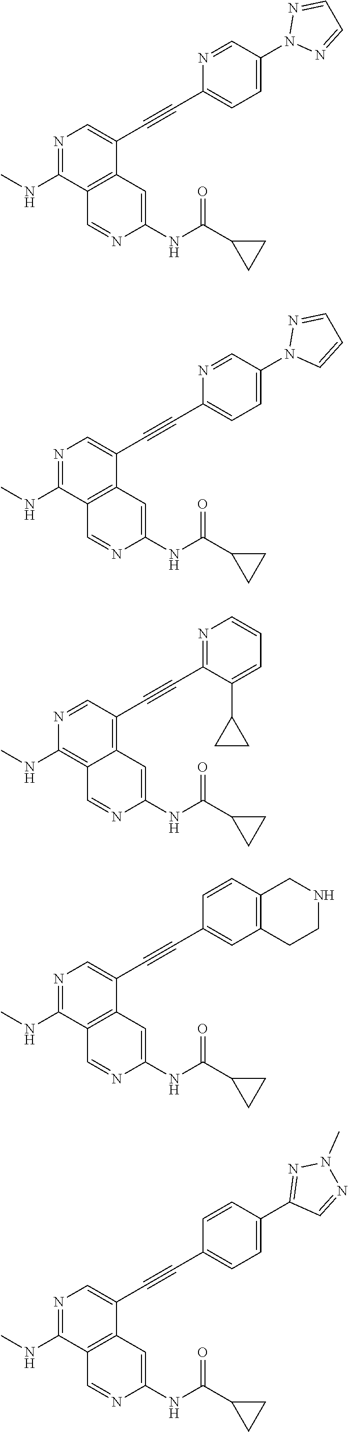

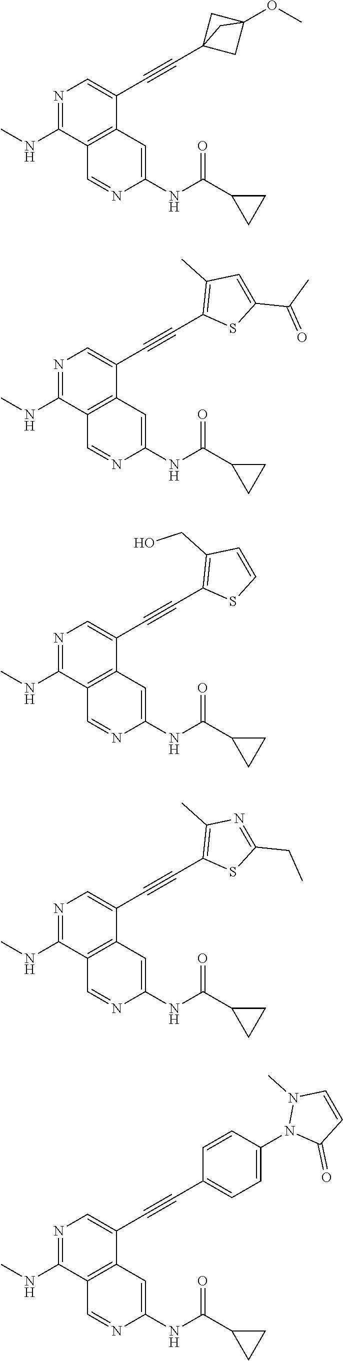

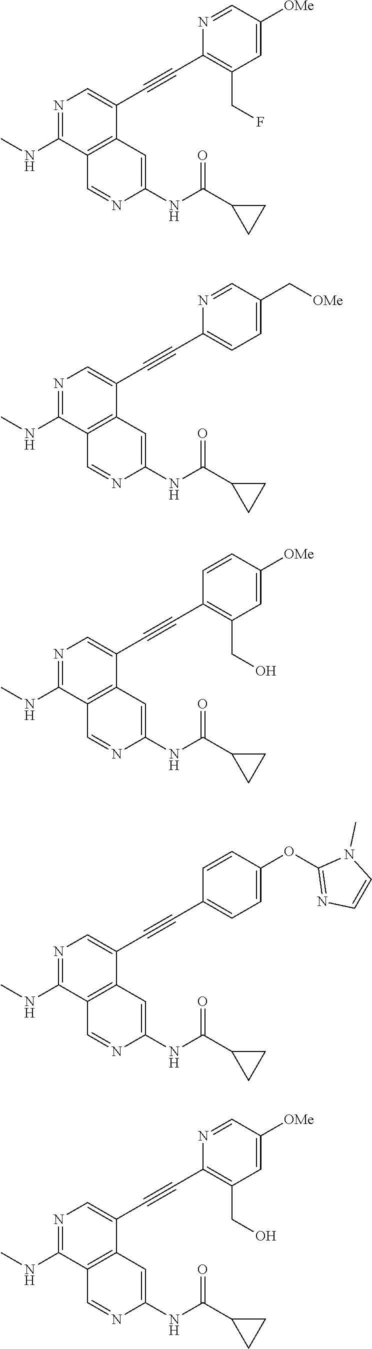

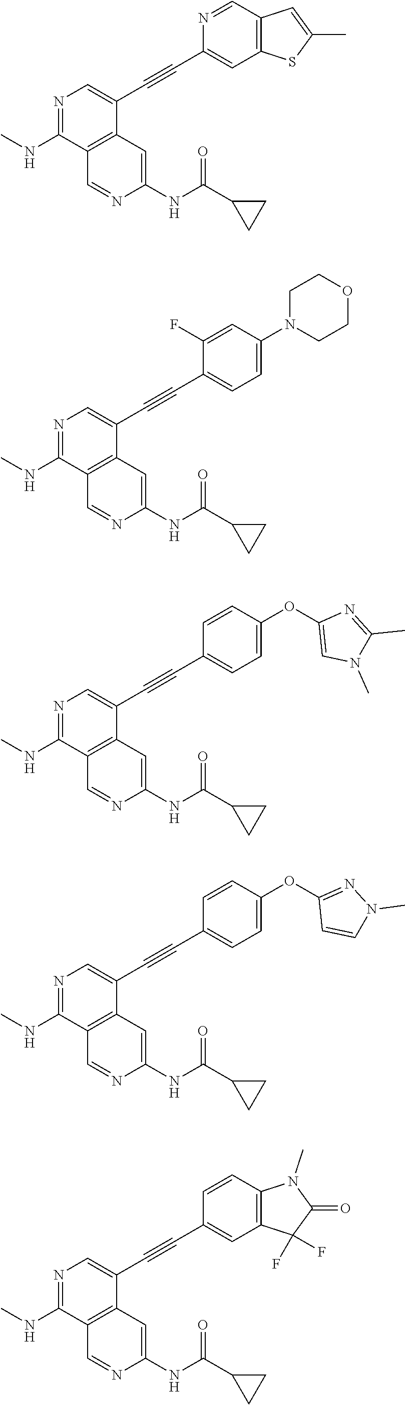

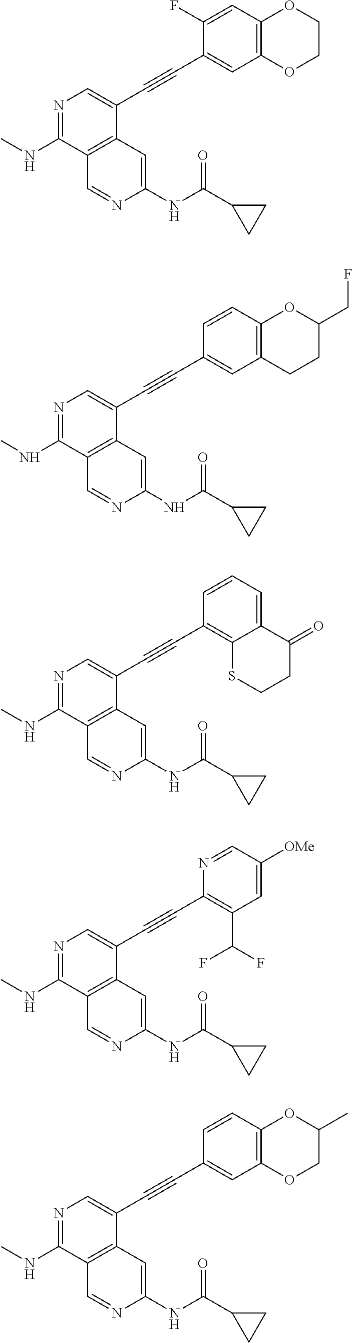

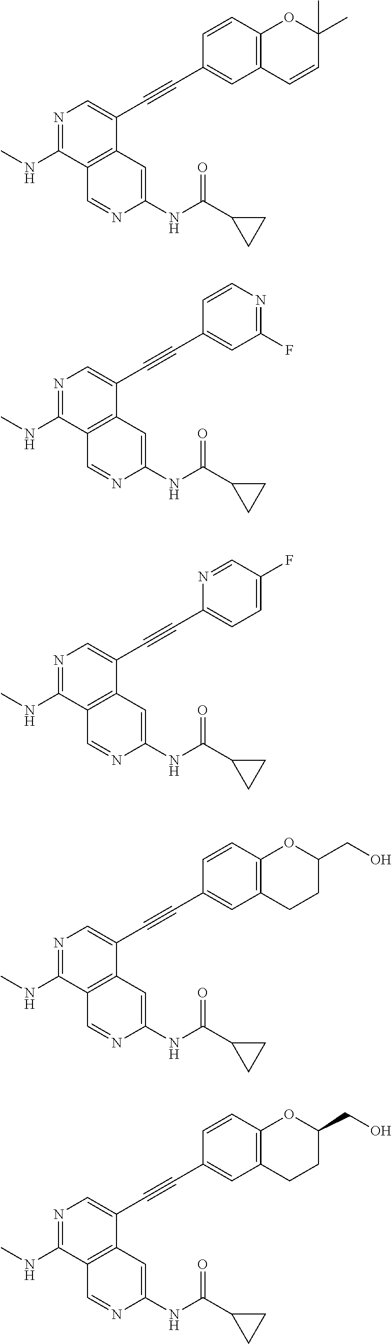

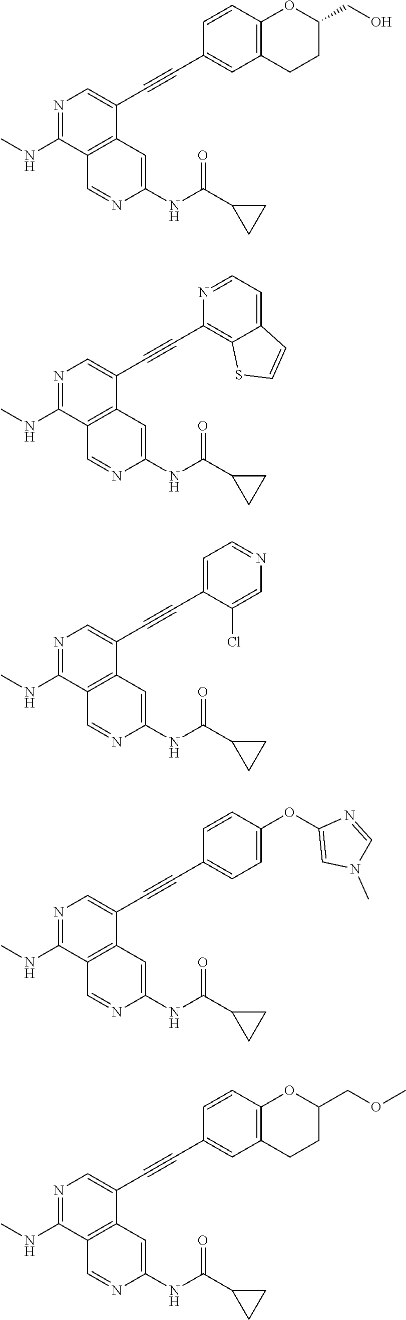

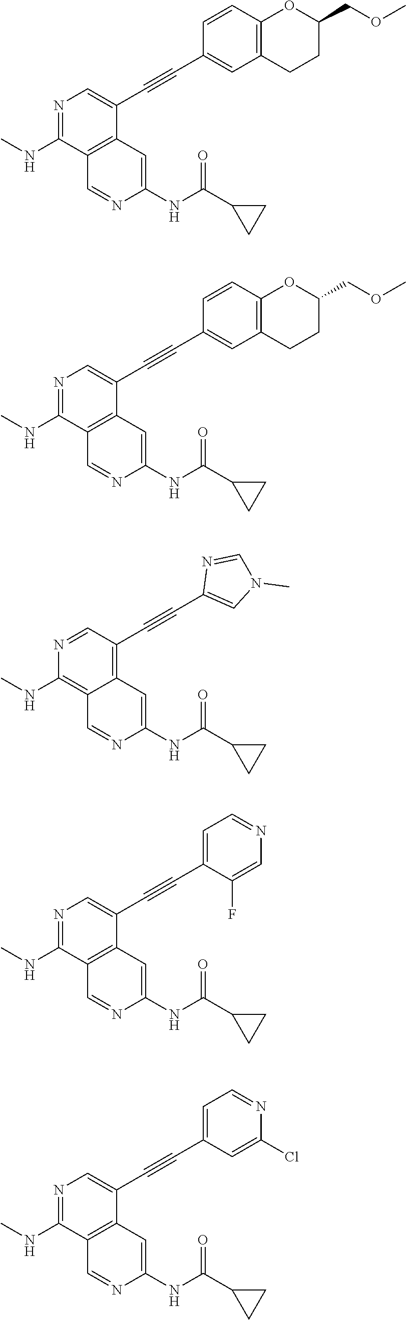

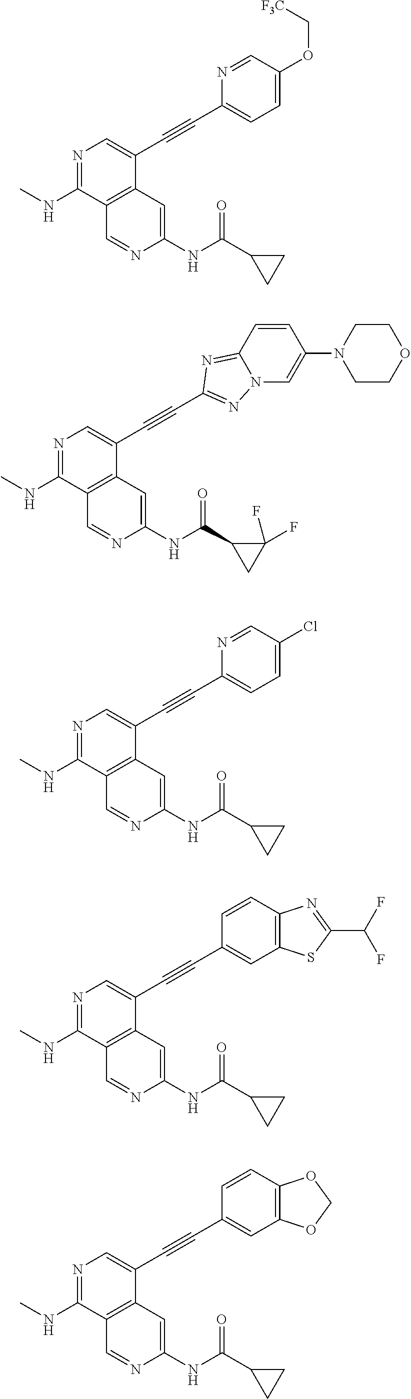

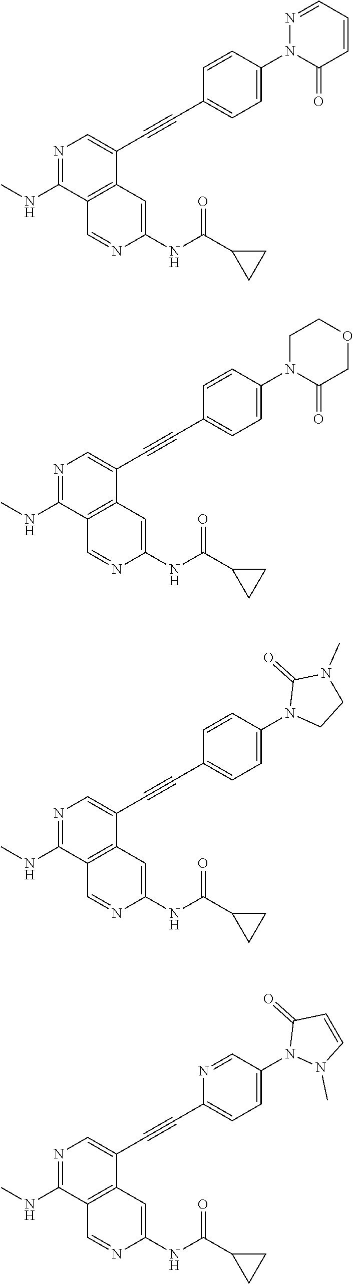 [SMILES: CNc1ncc(C#Cc2ccc(-n3ncccc3=O)cc2)c2cc(NC(=O)C3CC3)ncc12 ; CNc1ncc(C#Cc2ccc(N3CCOCC3=O)cc2)c2cc(NC(=O)C3CC3)ncc12 ; CNc1ncc(C#Cc2ccc(N3CCN(C)C3=O)cc2)c2cc(NC(=O)C3CC3)ncc12 ; CNc1ncc(C#Cc2ccc(-n3c(=O)ccn3C)cn2)c2cc(NC(=O)C3CC3)ncc12]

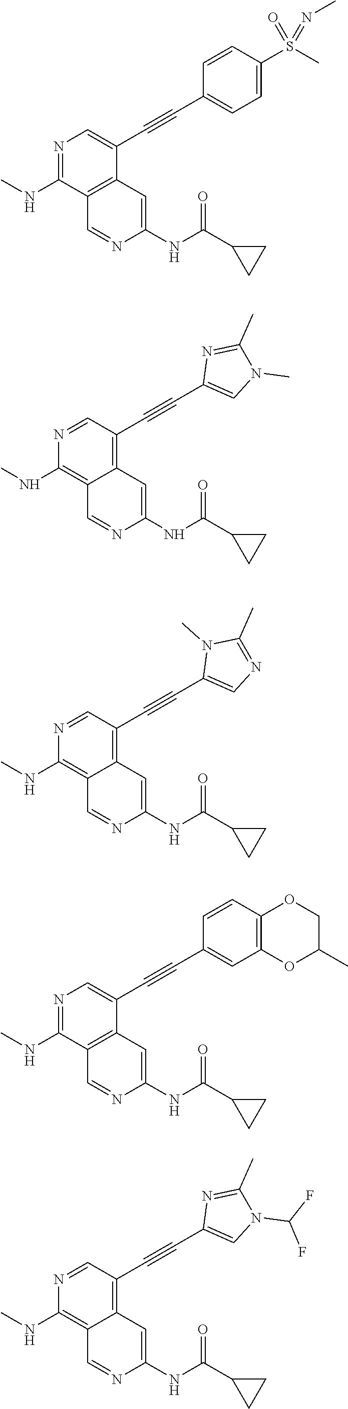

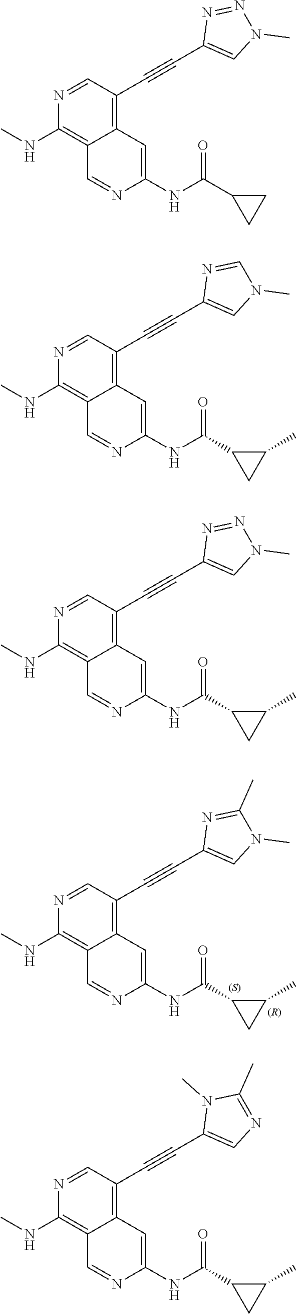

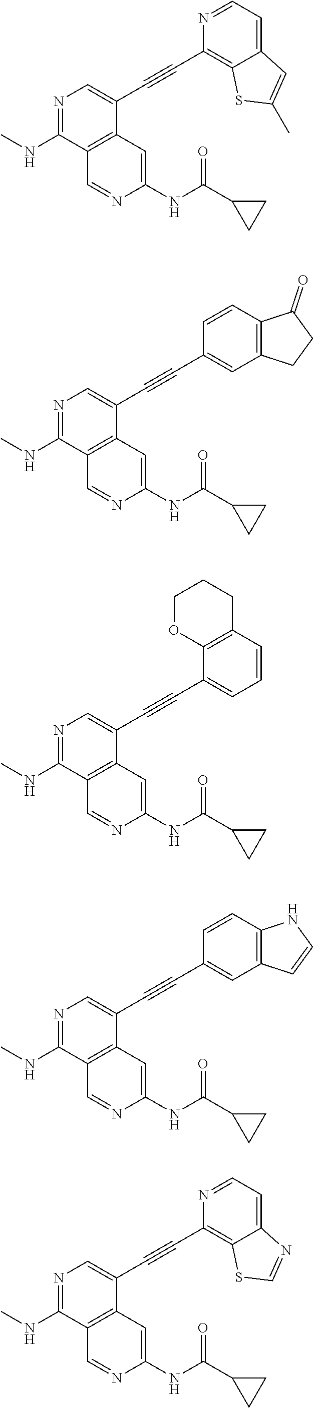

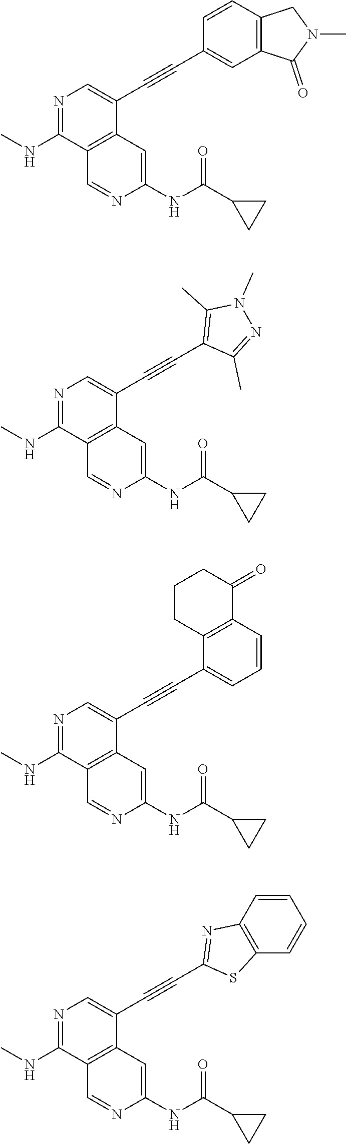

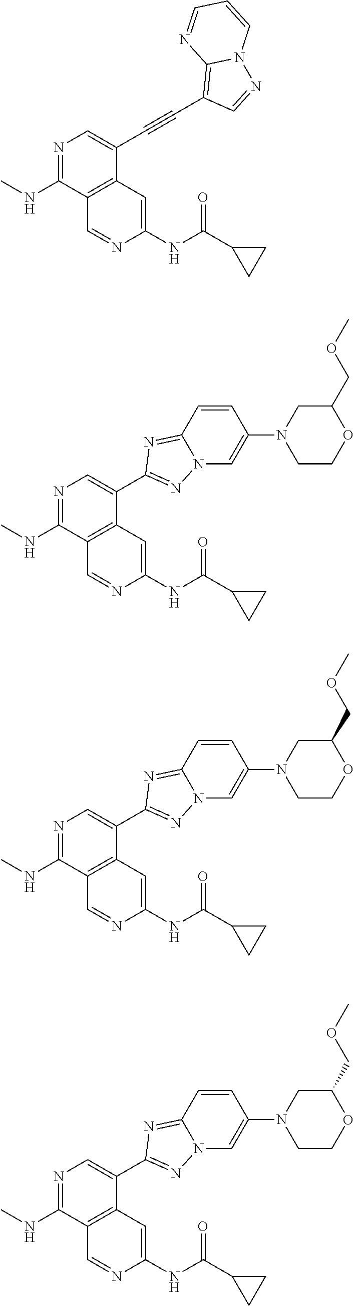

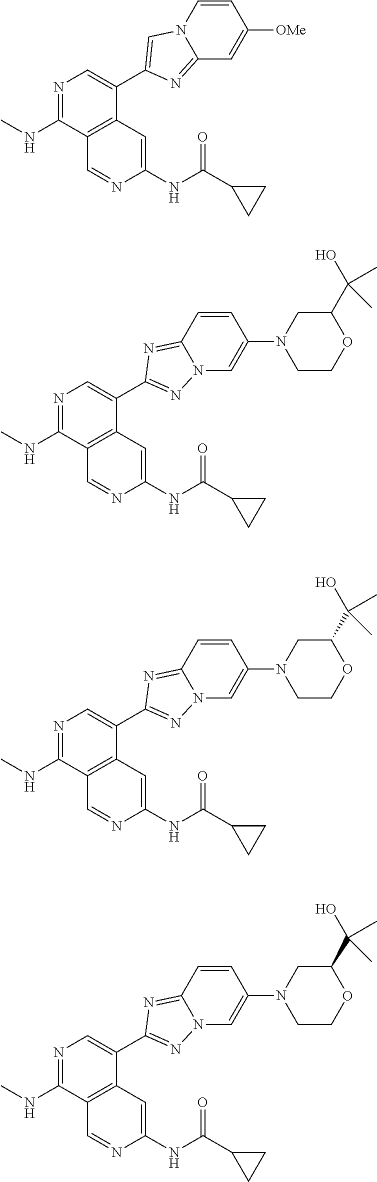

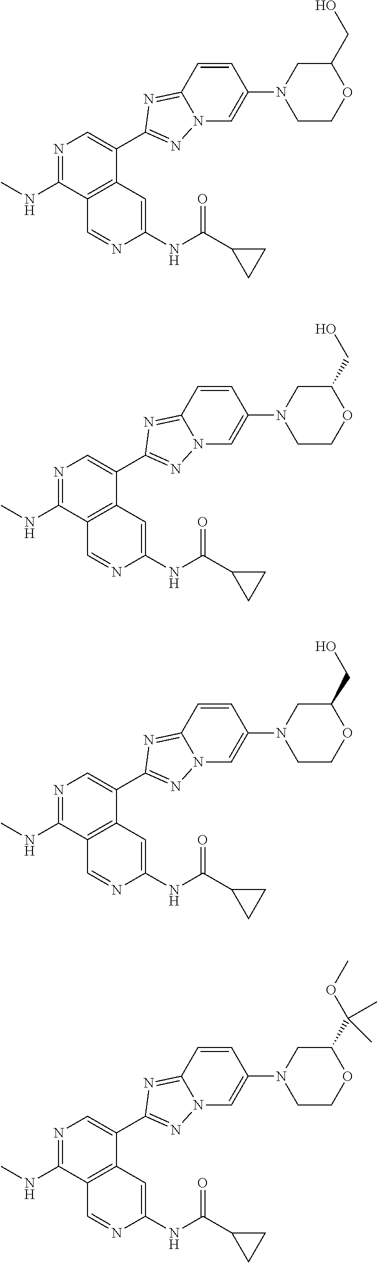 [SMILES: CNc1ncc(-c2nc3ccc(N4CCOC(CO)C4)cn3n2)c2cc(NC(=O)C3CC3)ncc12 ; CNc1ncc(-c2nc3ccc(N4CCO[C@@H](CO)C4)cn3n2)c2cc(NC(=O)C3CC3)ncc12 ; CNc1ncc(-c2nc3ccc(N4CCO[C@H](CO)C4)cn3n2)c2cc(NC(=O)C3CC3)ncc12 ; CNc1ncc(-c2nc3ccc(N4CCO[C@@H](C(C)(C)OC)C4)cn3n2)c2cc(NC(=O)C3CC3)ncc12]

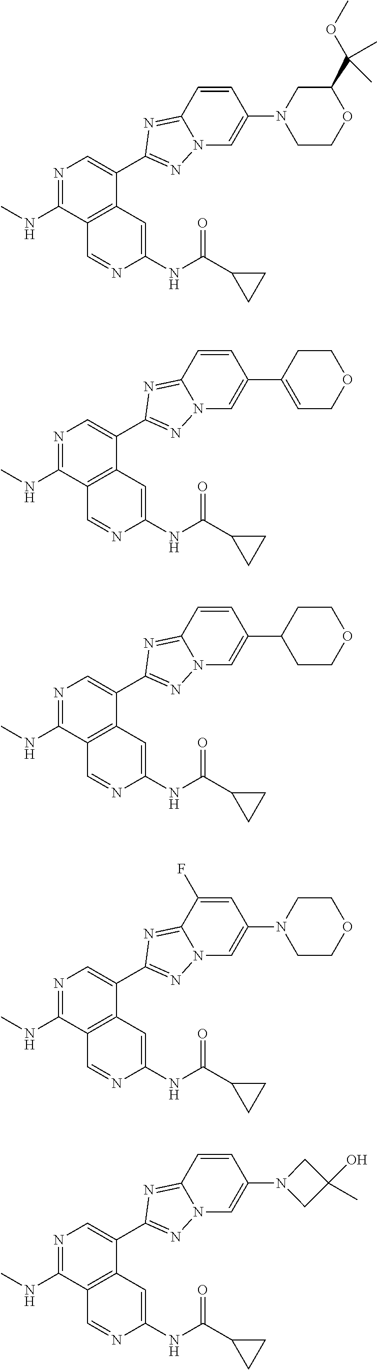

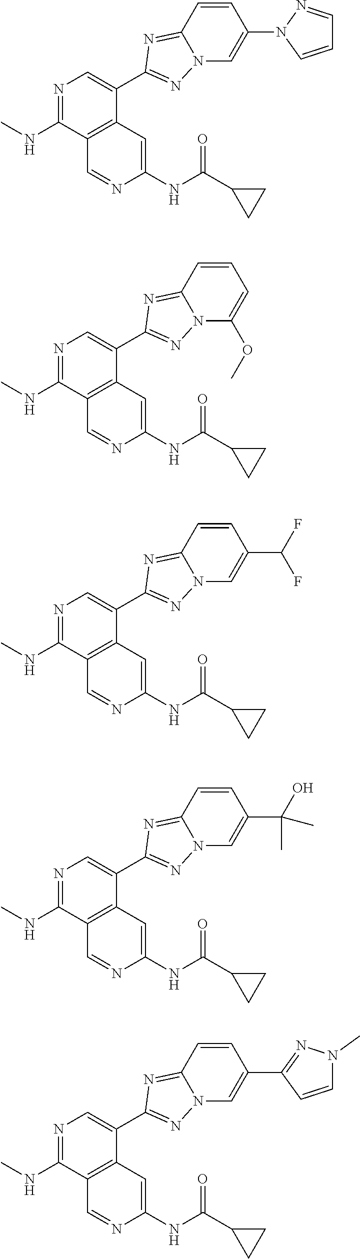

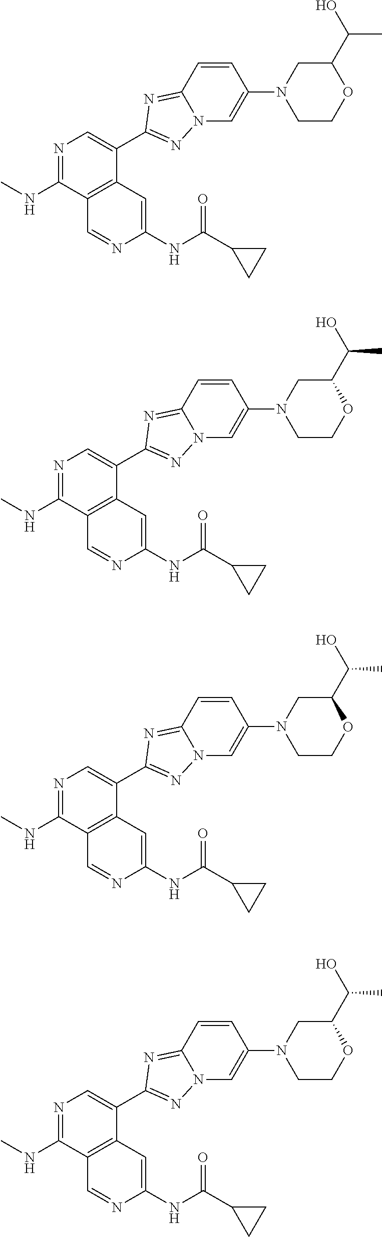

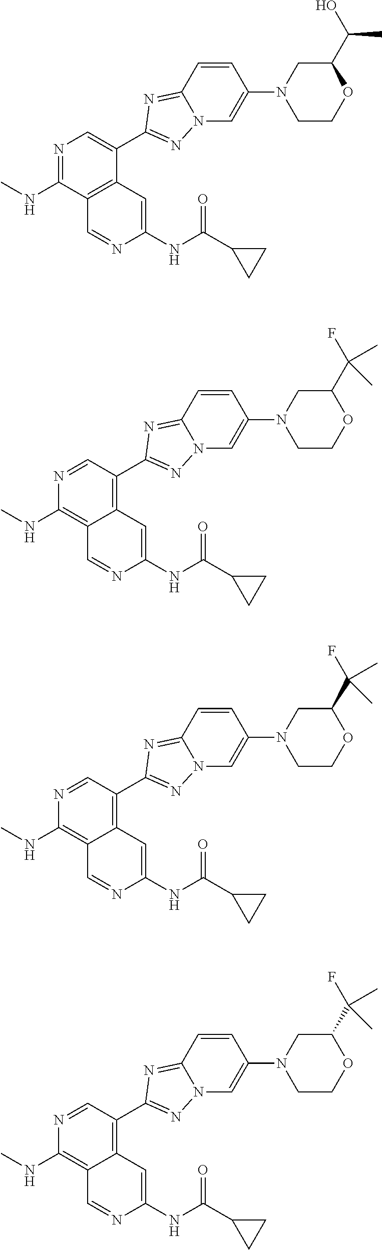

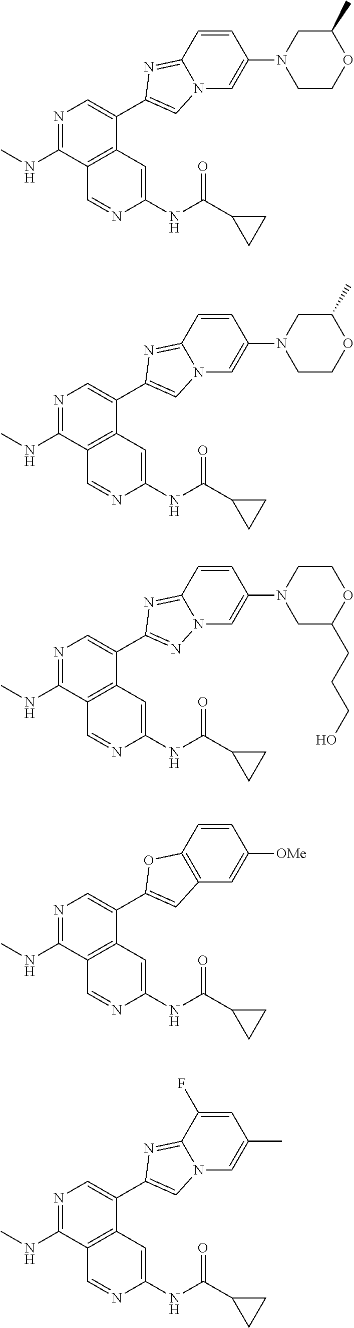

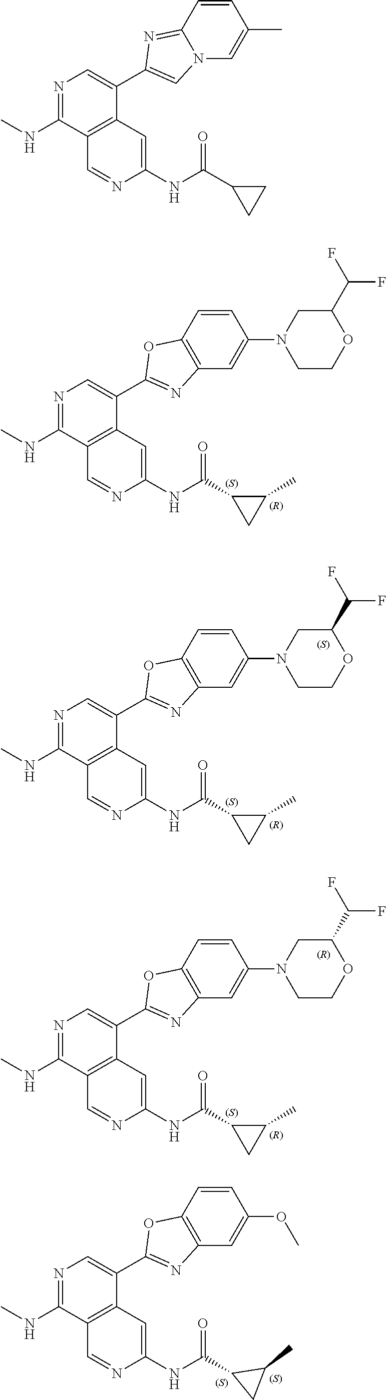

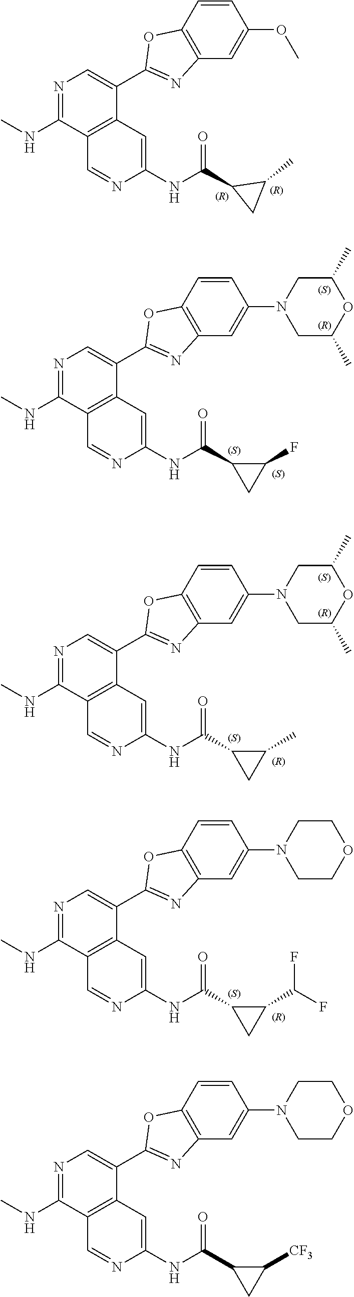

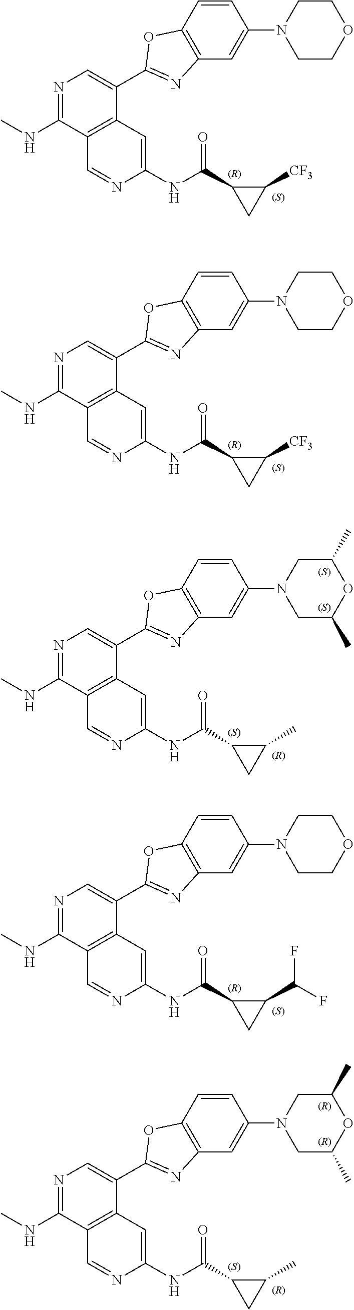

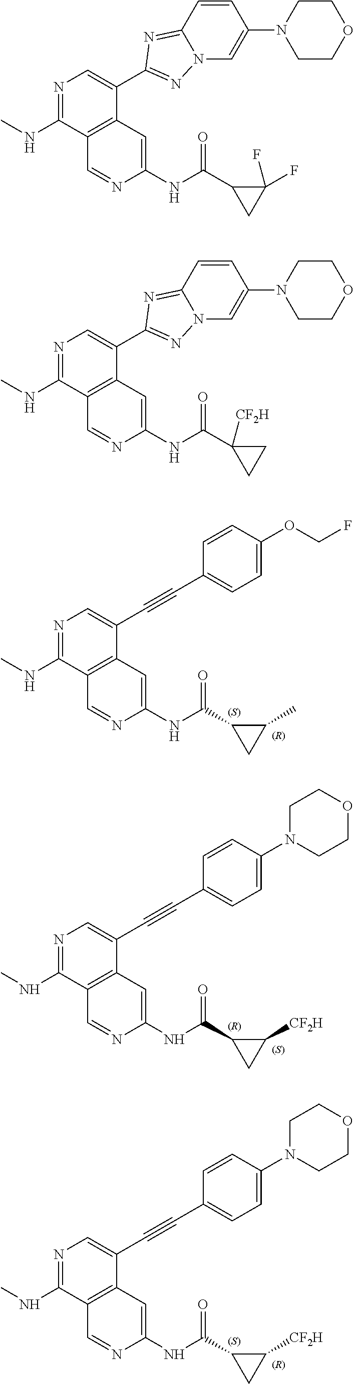

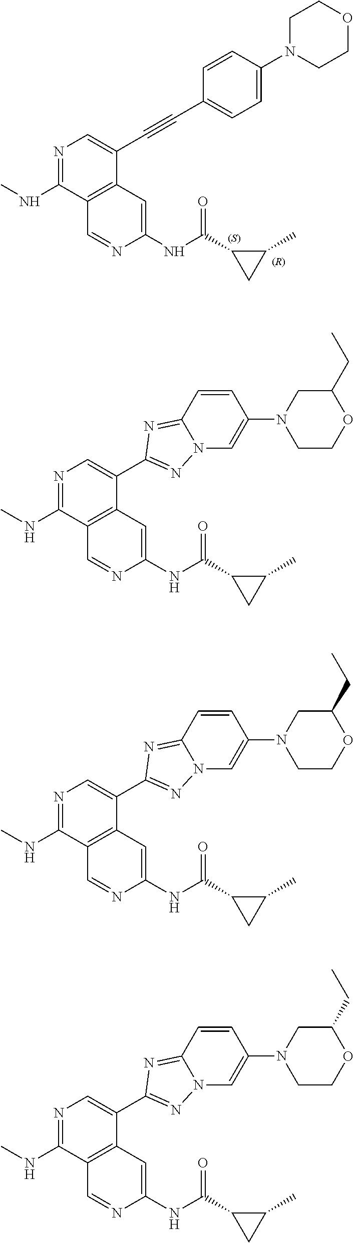

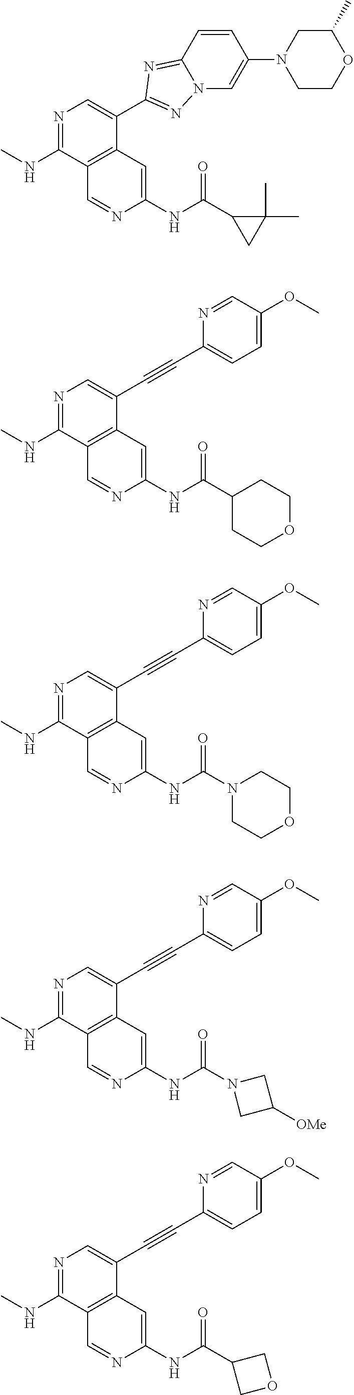

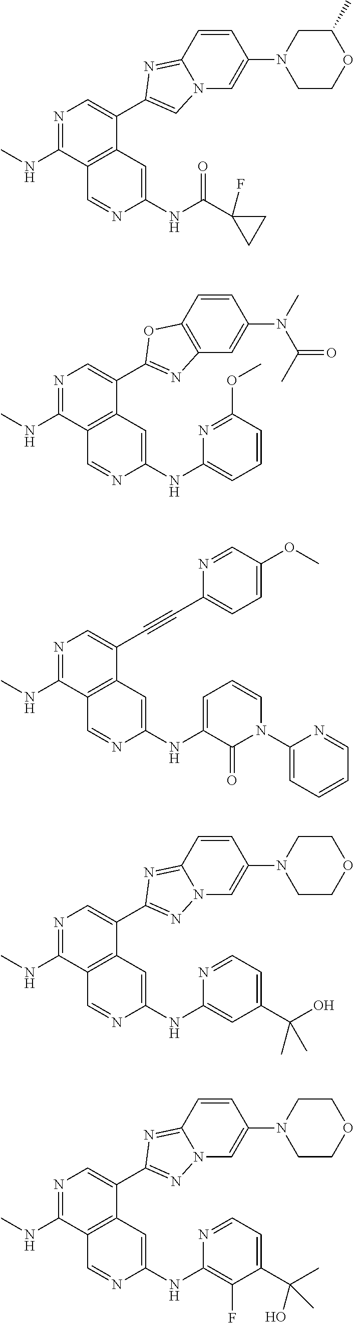

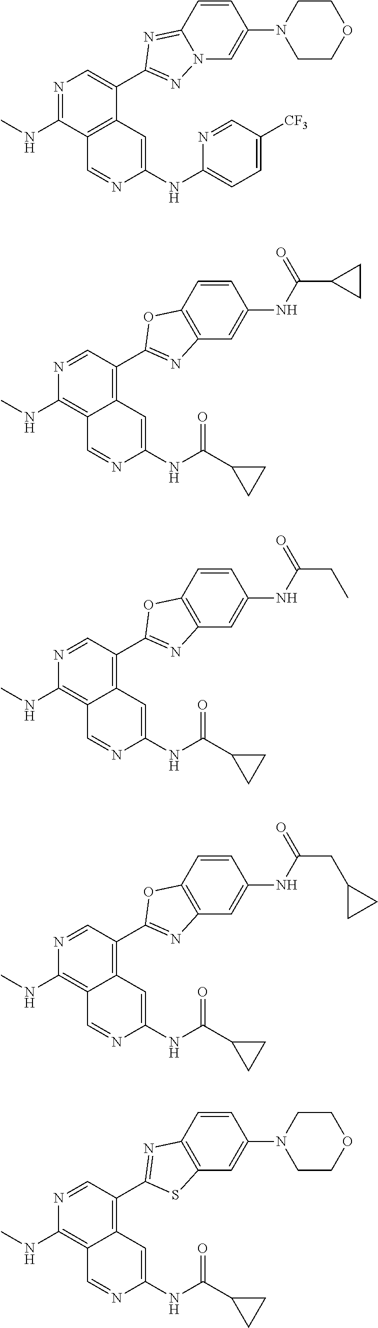

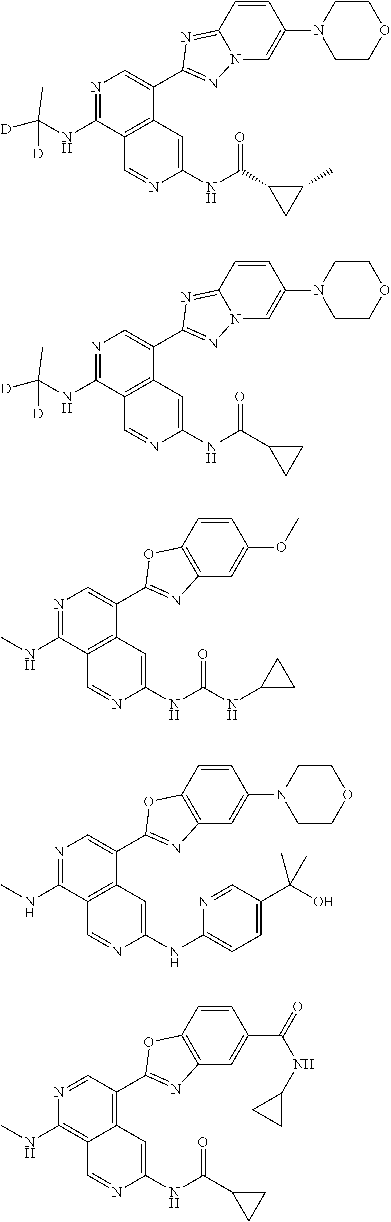

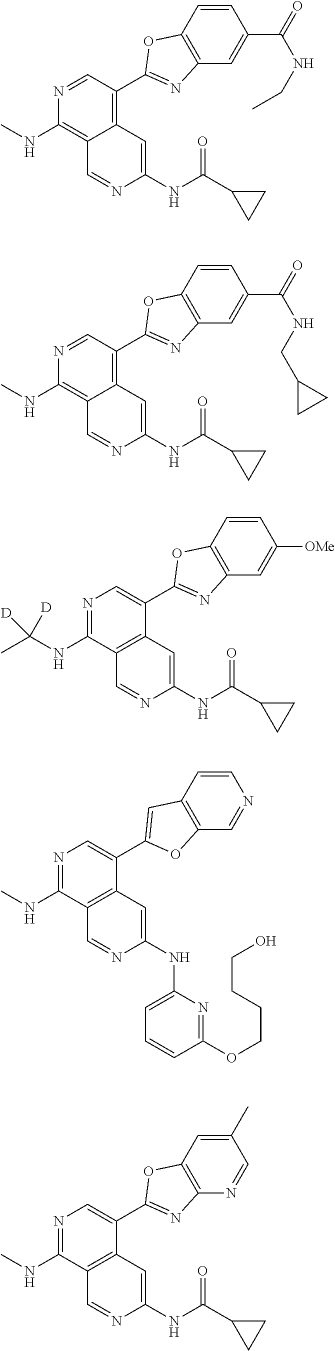

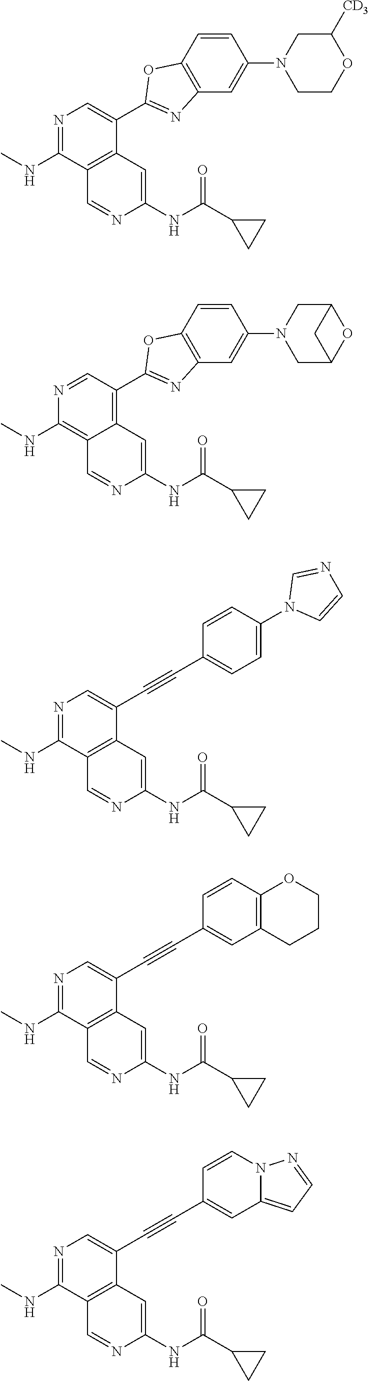

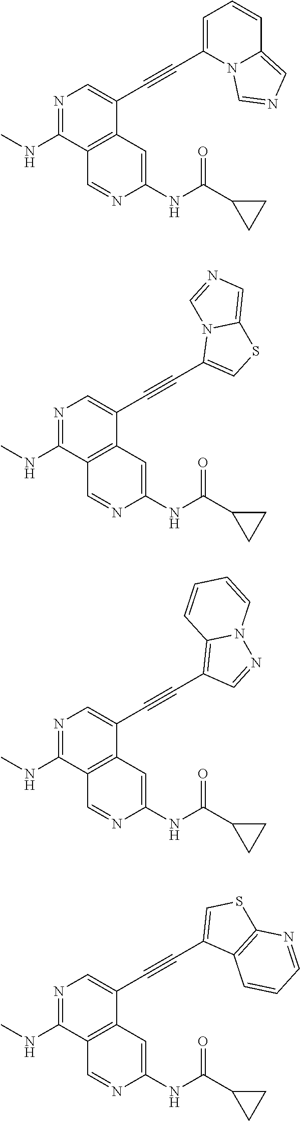

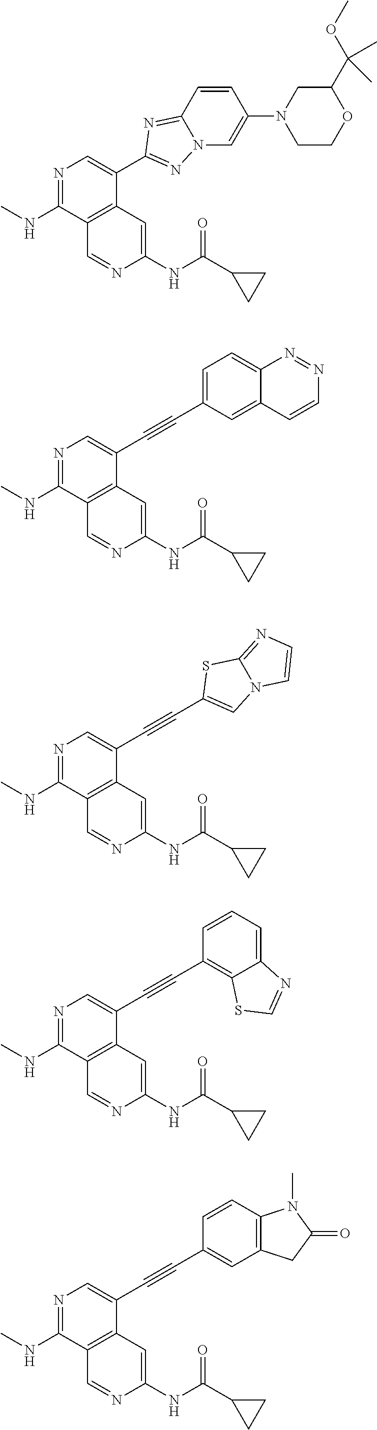

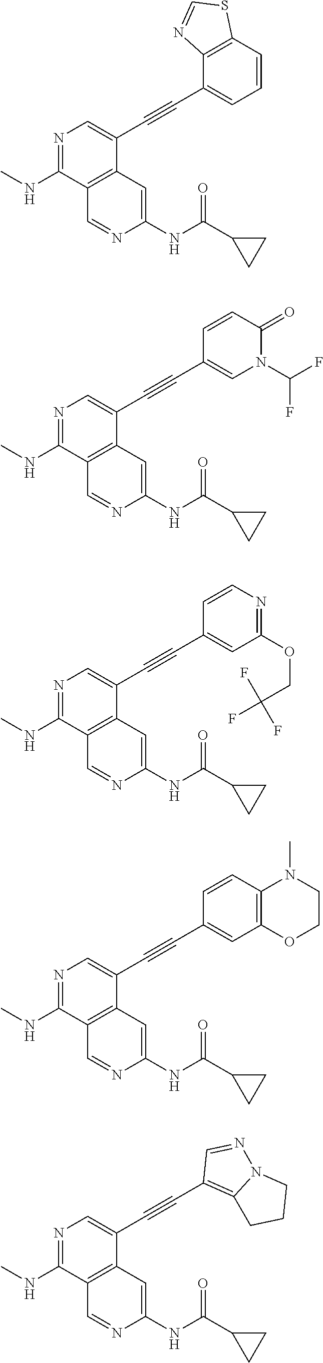

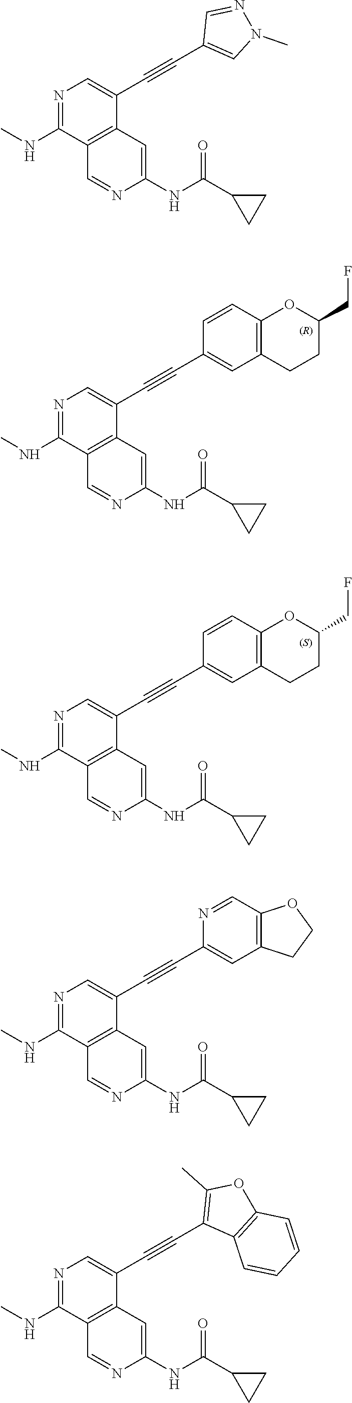

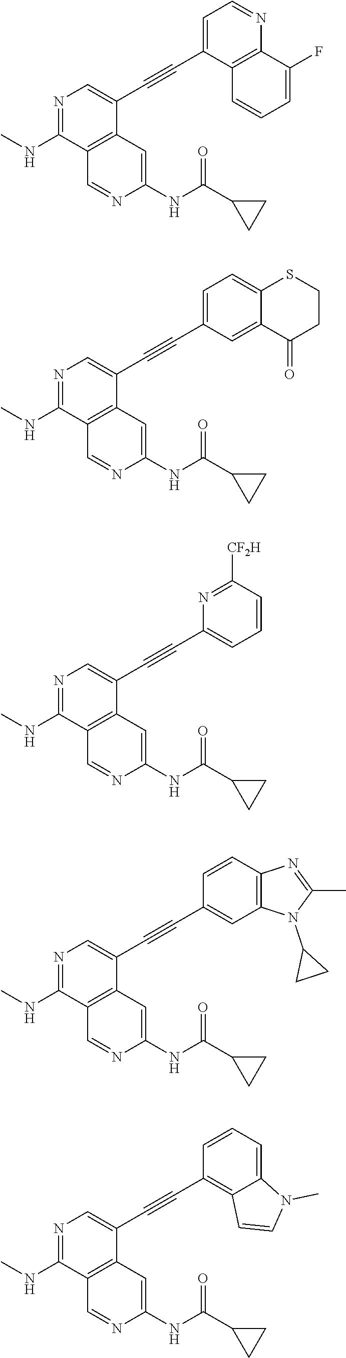

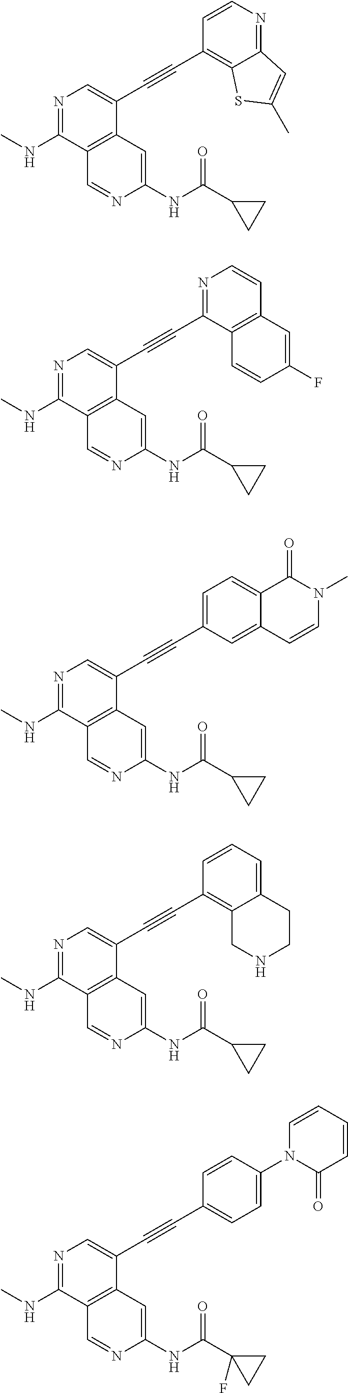

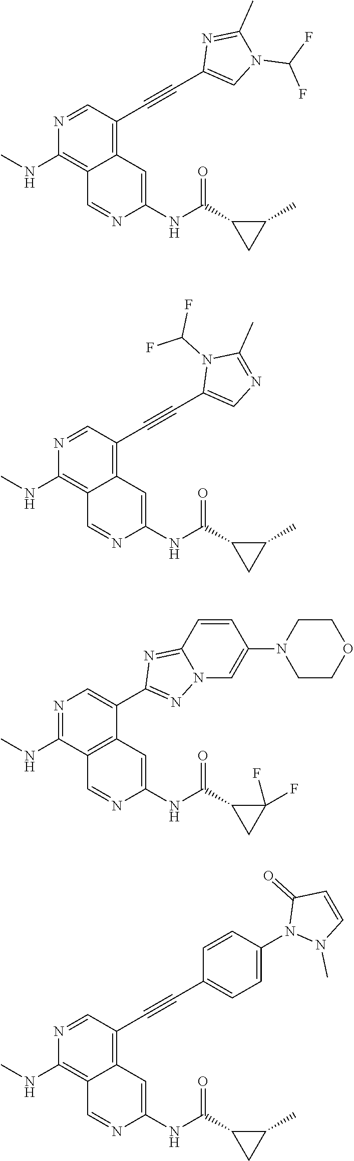

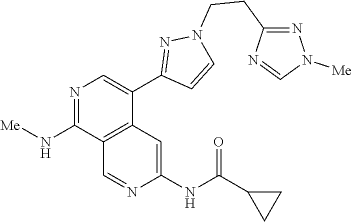 [SMILES: CNc1ncc(-c2ccn(CCc3ncn(C)n3)n2)c2cc(NC(=O)C3CC3)ncc12]

### Pharmaceutical Compositions

The present invention provides pharmaceutical compositions containing one or more compounds described above, or a pharmaceutically acceptable ester, prodrug, hydrate, solvate or salt of such a compound, optionally in combination with a pharmaceutically acceptable carrier. The invention further provides such compounds for the preparation of a medicament for the treatment of one or more diseases mentioned herein.

A pharmaceutical composition may contain one or more compounds of the invention in a therapeutically effective amount. A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage may be adjusted to the individual requirements in each particular case including the specific compound being administered, the route of administration, the condition being treated, as well as the patient being treated.

Compositions of the invention may include a vehicle for delivery of one or more compounds of the invention. For example, the composition may contain particles, such as nanoparticles, microparticles, liposomes, micelles, and virus particles.

Examples of pharmacologically acceptable salts of sufficiently basic compounds of the invention are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound of the invention may form alkali or earth alkali metal salts, for example sodium, potassium, lithium, calcium or magnesium salts; ammonium salts; or organic base salts, for example methyl amine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of the invention. Compounds of the invention may be solvated, especially hydrated. The hydratization/hydration may occur during the process of production or as a consequence of the hygroscopic nature of the initially water free compounds of the invention. The solvates and/or hydrates may e.g. be present in solid or liquid form.

It should be appreciated that certain compounds of the invention may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). All these tautomeric forms, geometrical or optical isomers (as well as racemates and diastereomers) and polymorphous forms are included in the invention. Since the compounds of the invention may contain asymmetric C-atoms, they may be present either as achiral compounds, mixtures of diastereomers, mixtures of enantiomers or as optically pure compounds. The present invention comprises both all pure enantiomers and all pure diastereomers, and also the mixtures thereof in any mixing ratio.

According to a further embodiment of the present invention, one or more hydrogen atoms of the compounds of the present invention may be replaced by deuterium. Deuterium modification improves the metabolic properties of a drug with little or no change in its intrinsic pharmacology. Deuterium substitution at specific molecular positions improves metabolic stability, reduces formation of toxic metabolites and/or increases the formation of desired active metabolites. Accordingly, the present invention also encompasses the partially and fully deuterated compounds of the invention. The term hydrogen also encompasses deuterium.

The therapeutic use of compounds according to the invention, their pharmacologically acceptable salts, solvates and hydrates, respectively, as well as formulations and pharmaceutical compositions also lie within the scope of the present invention. The pharmaceutical compositions according to the present invention may comprise at least one compound of the invention as an active ingredient and, optionally, carrier substances and/or adjuvants.

The present invention also relates to prodrugs which are composed of a compound of the invention and at least one pharmacologically acceptable protective group which will be cleaved off under physiological conditions, such as an alkoxy-, arylalkyloxy-, acyl-, acyloxymethyl group (e.g. pivaloyloxymethyl), an 2-alkyl-, 2-aryl- or 2-arylalkyl oxycarbonyl-2-alkylidene ethyl group or an acyloxy group as defined herein, e.g. ethoxy, benzyloxy, acetyl or acetyloxy or, especially for a compound of the invention, carrying a hydroxy group (—OH): a sulfate, a phosphate (—OPO₃ or —OCH₂OPO₃) or an ester of an amino acid. For example, compositions may contain pro-drugs of the hydroxy group of a compound of the invention.

As used herein, the term pharmaceutically acceptable ester especially refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and aikanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The present invention also relates to a prodrug, a biohydrolyzable ester, a biohydrolyzable amide, a polymorph, tautomer, stereoisomer, metabolite, N-oxide, biohydrolyzable carbamate, biohydrolyzable ether, physiologically functional derivative, atropisomer, or in vivo-hydrolysable precursor, diastereomer or mixture of diastereomers, chemically protected form, affinity reagent, complex, chelate and a stereoisomer of the compounds of the invention.

As mentioned above, therapeutically useful agents that contain compounds of the invention, their solvates, salts or formulations are also comprised in the scope of the present invention. In general, compounds of the invention will be administered by using the known and acceptable modes known in the art, either alone or in combination with any other therapeutic agent.

For oral administration such therapeutically useful agents can be administered by one of the following routes: oral, e.g. as tablets, dragees, coated tablets, pills, semisolids, soft or hard capsules, for example soft and hard gelatin capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatin capsules, the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Particularly useful are lipids, such as phospholipids (e.g., natural origin and/or with a particle size between 300 to 350 nm) in phosphate buffered saline (pH=7 to 8, e.g., 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 10 mg to about 10,000 mg, or from about 20 mg to about 1,000 mg, should be appropriate, although the upper limit may be exceeded when indicated. The daily dosage can be administered as a single dose or in divided doses, or for parenteral administration, it may be given as continuous infusion or subcutaneous injection,

### Methods of Making Compounds

The invention also provides methods of making compounds of the invention, such as those described above. Synthesis schemes for making specific compounds of Formula (I) are provided in the Examples below.

### Methods of Treating Conditions

The compounds and compositions of the invention modulate activity of one or more protein kinases. The compounds and compositions may inhibit, activate, or otherwise alter kinase activity. Consequently, the compounds and compositions may be used to diagnose, treat, or prevent a condition, such as a disease, disorder, or other condition for which modulation of kinase activity provides therapeutic benefit.

Diseases, disorders, and conditions that can be diagnosed and/or treated using compositions and methods of the invention include those associated with aberrant activity, e.g., increased activity, or decreased activity, of one or more kinases. The kinase may be a serine-threonine kinase or a tyrosine kinase, e.g., a receptor tyrosine kinase or non-receptor tyrosine kinase. The kinase may be a member of the JAK family. For example and without limitation, the kinase may be death-associated protein kinase 1 (DAPK1), leucine-rich repeat kinase 2 (LRRK2), NUAK family SNF1-like kinase 1 (NUAK1, also known as AMPK-related protein kinase 5 or ARKS), spleen tyrosine kinase (SYK), or non-receptor tyrosine-protein kinase TYK2 (TYK2), including mutants of any, of the aforementioned kinases.

The disease, disorder, or condition may be associated with aberrant DAPK1 activity, such as Alzheimer's disease, atherosclerosis, brain injury, breast cancer, such as triple negative breast cancer, cancer, ceramide and glutamate toxicity, drug resistance, e.g., resistance to cancer drugs, epilepsy, heart failure, ischemia, myofibrial degeneration, neurodegenerative disease, seizure, tumor metastasis, tumor suppression, ulcerative colitis, or viral infection.

The disease, disorder, or condition may be associated with aberrant LRRK2 activity, such as Alzheimer's disease, Crohn's disease, inflammatory bowel disease, an inflammatory disease, leprosy, neurodegenerative diseases, a non-skin cancer, or Parkinson's disease, including familial Parkinson's disease, sporadic Parkinson's disease, late-onset Parkinson's disease (PD), and type 8 Parkinson's disease.

The disease, disorder, or condition may be associated with aberrant MAKI activity, such as cancer, e.g., colorectal cancer, stomach cancer, endometrial cancer, or multiple myeloma, diabetes, fibrosis, a neurodegenerative diseases, or omphalocele.

The disease, disorder, or condition may be associated with aberrant SYK activity, such as an allergic disorder, anaphylactic shock, aneurysm, arteriosclerosis, asthma, an autoimmune disease, B-cell lymphoma, breast cancer, breast ductal carcinoma in situ (BCIS), chronic lymphocytic leukemia (CLL), diffuse large B cell lymphoma (DLBCL), eosinophilic inflammation, episcleritis, follicular lymphoma, a functional gastrointestinal disorder, fungal keratitis, gastric cancer, head and neck cancer, heart attack, hemolytic anemia, heparin-induced thrombocytopenia, immune thrombocytopenia purpura, infection, e.g., fungal, viral, or bacterial infection, keratoconjunctivitis sicca, mantle Cell Lymphoma (MCL), multiple sclerosis, myelodysplastic syndrome, myocardial infarction, Nasopharyngeal carcinoma, non-Hodgkins lymphoma, rheumatoid arthritis, scleritis, stroke, or systemic lupus.

The disease, disorder, or condition may be associated with aberrant TYK2 activity, such as autoimmune disorders. Crohn's disease, hyperimmunoglobulin E syndrome, inflammatory bowel disease, multiple sclerosis (MS), multiple sclerosis (MS), psoriasis, rheumatoid arthritis, systemic lupus erythematosus (SLE), type 1 diabetes (TIT), or ulcerative colitis.

The disease, disorder, or condition may be or include a respiratory tract/obstructive airways disease or disorder, such as rhinorrhea, tracheal constriction, airway contraction, acute-, allergic, atrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), pollinosis, asthma (such as bronchial, atopic, allergic, intrinsic, extrinsic, exercise-induced, cold air-induced, occupational, bacterial infection-induced, and dust asthma particularly chronic or inveterate asthma (e.g. late asthma and airways hyper-responsiveness)), bronchitis (including chronic, acute, arachidic, catarrhal, croupus, phthinoid and eosinophilic bronchitis), cardiobronchitis, pneumoconiosis, chronic inflammatory disease of the lung which result in interstitial fibrosis, such as interstitial lung disease (ILD) (e.g., idiopathic pulmonary fibrosis, or ILD associated with rheumatoid arthritis, or other autoimmune conditions), acute lung injury (ALI), adult respiratory distress syndrome (ARDS), chronic obstructive pulmonary, airways or lung disease (CORD, COAD, COLD or COPD, such as irreversible COPD), chronic sinusitis, conjunctivitis (e.g. allergic conjunctivitis), cystic fibrosis, extrinsic allergic alveolitis (like farmer's lung and related diseases), fibroid lung, hypersensitivity lung diseases, hypersensitivity pneumonitis, idiopathic interstitial pneumonia, nasal congestion, nasal polyposis, otitis media, and cough (chronic cough associated with inflammation or iatrogenic induced), pleurisy, pulmonary congestion, emphysema, bronchiectasis, sarcoidosis, lung fibrosis, including cryptogenic fibrosing alveolitis, fibrosis complicating anti-neoplastic therapy and chronic infection, including tuberculosis and aspergillosis and other fungal infections, vasculitic and thrombotic disorders of the lung vasculature, and pulmonary hypertension, acute viral infection including the common cold, and infection due to respiratory syncytial virus, influenza, coronavirus (including SARS) and adenovirus, allergic bronchopulmonary mycosis, emphysema, diffuse panbronchiolitis, systemic anaphylaxis or hypersensitivity responses, drug allergies (e.g., to penicillin, cephalosporins), insect sting allergies, and food related allergies which may have effects remote from the gut (such as migraine, rhinitis and eczema), anaphylactic shock, or vascular spasms.

The disease, disorder, or condition may be or include a bone and joint related disease or disorder, such as osteoporosis, arthritis (including rheumatic, infectious, autoimmune, chronic, malignant), seronegative spondyloarthropathies (such as ankylosing spondylitis, rheumatoid spondylitis, psoriatic arthritis, enthesopathy, Bechet's disease, Marie-Strumpell arthritis, arthritis of inflammatory bowel disease, and Reiter's disease), systemic sclerosis, osteoarthritis, osteoarthrosis, both primary and secondary to e.g. congenital hip dysplasia, cervical and lumbar spondylitis, and low back and neck pain, Still's disease, reactive arthritis and undifferentiated spondarthropathy, septic arthritis and other infection-related arthropathies and bone disorders such as tuberculosis, including Pott's disease and Poncet's syndrome, acute and chronic crystal-induced synovitis including urate gout, calcium pyrophosphate deposition disease, and calcium apatite related tendon, bursar and synovial inflammation, primary and secondary Sjogren's syndrome, systemic sclerosis and limited scleroderma, mixed connective tissue disease, and undifferentiated connective tissue disease, inflammatory myopathies including, polymalgia rheumatica, juvenile arthritis including idiopathic inflammatory arthritides of whatever joint distribution and associated syndromes, other joint disease (such as intervertebral disc degeneration or temporomandibular joint degeneration), rheumatic fever and its systemic complications, vasculitides including giant cell arteritis, Takayasu's arteritis, polyarteritis nodosa, microscopic polyarteritis, and vasculitides to associated with viral infection, hypersensitivity reactions, cryoglobulins, paraproteins, low back pain, Familial Mediterranean fever, Muckle-Wells syndrome, and Familial Hibenian Fever, Kikuchi disease, drug-induced arthalgias, tendonititides, polychondritis, and myopathies, osteoporosis, osteomalacia like osteoporosis, osteopenia, osteogenesis imperfecta, osteopetrosis, osteofibrosis, osteonecrosis, Paget's disease of bone, hypophosphatemia, Felty's syndrome, Still's disease, slack of artificial joint implant, sprain or strain of muscle or joint, tendinitis, fasciitis, periarthritis humeroscapularis, cervico-omo-brachial syndrome, or tenosynovitis.

The disease, disorder, or condition may be or include a skin or eye related disease or disorder, such as glaucoma, ocular hypertension, cataract, retinal detachment, psoriasis (including psoriasis vulgaris, pustular psoriasis, arthritic psoriasis, erythroderma psoriaticum), palmoplantar pustulosis, xerodoma, eczematous diseases (like atopic dermatitis, ultraviolet radiation dermatitis, contact dermatitis, and seborrheic dermatitis), phytodermatitis, photodermatitis, cutaneous eosinophilias, chronic skin ulcers, cutaneous lupus erythematosus, contact hypersensitivity/allergic contact dermatitis (including sensitivity to poison ivy, sumac, or oak), and eosinophilic folliculitis (Ofuji's disease), pruritus, drug eruptions, urticaria (acute or chronic, allergic or non-allergic), acne, erythema, dermatitis herpetiformis, scleroderma, vitiligo, lichen planus, lichen sclerosus et atrophica, pyodenna gangrenosum, skin sarcoid, pemphigus, ocular pemphigus, pemphigoid, epidermolysis bullosa, angioedema, vasculitides, toxic erythemas, cutaneous eosinophilias, alopecia areata, male-pattern baldness, Sweet's syndrome, Stevens-Johnson syndrome, Weber-Christian syndrome, erythema, multiforme, cellulitis, both, infective and non infective, panniculitis, cutaneous Lymphomas, non¬melanoma skin cancer and other dysplastic lesions, blepharitis, iritis, anterior and posterior uveitis, choroiditis, autoimmune, degenerative or inflammatory disorders affecting the retina, ophthalmitis including sympathetic ophthalmitis, sarcoidosis, xerosis infections including viral, fungal, and bacterial, allergic conjunctivitis, increased fibrosis, keloids, keloplasty, post surgical scars, epidermolysis bullosa, dry eye, ocular inflammation, allergic conjunctivitis, venial conjunctivitis, vernal keratoconjunctivitis, and giant papillary conjunctivitis, ocular angiogenesis, cornea damage and scar, all forms of macular degeneration, macular edema, macular dystrophy, abnormal wound healing, scleritis, episcleritis, pachydermia, peripheral ulcerative keratitis, fungal keratitis, herpetic keratitis, invasive aspergillosis; conical cornea, dystorphia epithelialis comeae, or severe intraocular inflammation.

The disease, disorder, or condition may be or include a gastrointestinal tract and abdominal related disease or disorder, such as celiac/coeliac disease (e.g. celiac sprue), cholecystitis, enteritis (including infectious, ischemic, radiation, drug-induced, and eosinophilic gastroenteritis), eosinophilic esophagitis, eosinophilic gastrointestinal inflammation, allergen induced diarrhea, enteropathy associated with seronegative arthropathies, gastritis, autoimmune atrophic gastritis, ischemic bowel disease, inflammatory bowel disease (Crohn's disease and ulcerative colitis), colitis, Mooren's ulcer, irritable bowel syndrome, necrotizing enterocolitis, gut ischemia, glossitis, gingivitis, periodontitis, oesophagitis, including reflex, proctitis, fibrosis and cirrhosis of the liver, pancreatitis, both acute and chronic, pancreatic fibrosis, pancreatic sclerosis, pancreatolithiasis, hepatic cirrhosis, hepatitis (congestive, autoimmune, acute, fulminant, chronic, drug-induced, alcoholic, lupoid, steatohepatitis and chronic viral), fatty liver, primary biliary cirrhosis, hepatic porphyria, and gastrointestinal related allergic disorders, spastic colon, diverticulitis, gastroenteric bleeding, Behcet's disease; partial liver resection, acute liver necrosis (e.g. necrosis caused by toxins, viral hepatitis, shock or anoxia), or hemolytic uremic syndrome.

The disease, disorder, or condition may be or include a hematological disease or disorder, such as anemias, coagulation, myeloproliferative disorders, hemorrhagic disorders, leukopenia, eosinophilic disorders, leukemias (e.g. myelogenous, lymphomas, plasma cell dyscrasias, disorders of the spleen, Band's disease, hemophilia, purpura (including idiopathic thrombocytopenic purpura), or Wiskott-Aldrich syndrome.

The disease, disorder, or condition may be or include a metabolic disease or disorder, such as obesity, anyloidosis, disturbances of the amino and acid metabolism like branched chain disease, hyperaminoacidemia, hyperaminoaciduria, disturbances of the metabolism of urea, hyperammonemia, mucopolysaccharidoses e.g. Maroteaux-Lamy syndrome, storage disease like glycogen storage diseases and lipid storage diseases, glycogenosis I diseases like Cori's disease, malabsorption diseases like intestinal carbohydrate malabsorption; oligosaccharidase deficiency like maltase-, lactase-, sucrase-insufficiency, disorders of the metabolism of fructose, disorders of the metabolism of galactose, galactosaemia; disturbances of carbohydrate utilization like diabetes, hypoglycemia, disturbances of pyruvate metabolism, hypolipidemia, hypolipoproteinemia, hyperlipidemia, hyperlipoproteinemia, carnitine or carnitine acyltransferase deficiency, disturbances of the porphyrin metabolism, porphyrins, disturbances of the purine metabolism, lysosomal diseases, metabolic diseases of nerves and nervous systems like gangliosidoses, sphingolipidoses, sulfatidoses, leucodystrophies, or Lesch Nyhan syndrome.

The disease, disorder, or condition may be or include a cerebellar dysfunction or disturbance of brain metabolism, such as dementia, Alzheimer's disease, Huntington's chores, Parkinson's disease, Pick's disease, toxic encepha-lopathy, demyelinating neuropathies like inflammatory neuropathy, Guillain-Barre syndrome; Meniere's disease and radiculopathy, primary and secondary metabolic disorders associated with hormonal defects like any disorder stemming from either an hyperfunction or hypofunction of some hormone-secreting endocrine gland and any combination thereof. Sipple's syndrome, pituitary gland dysfunction and its effects on other endocrine glands; such as the thyroid, adrenals, ovaries, and testes, acromegaly, hyper- and hypothyroidism, euthyroid goiter, euthyroid sick syndrome, thyroiditis, and thyroid cancer, over or underproduction of the adrenal steroid hormones, adrenogenital syndrome, Cushing's syndrome, Addison's disease of the adrenal cortex, Addison's pernicious anemia, primary and secondary aldosteronism, diabetes insipidus, diabetes mellitus, carcinoid syndrome, disturbances caused by the dysfunction of the parathyroid glands, pancreatic islet cell dysfunction, diabetes, disturbances of the endocrine system of the female like estrogen deficiency, resistant ovary syndrome; muscle weakness, myotonia. Duchenne's and other muscular dystrophies, dystrophia myotonica of Steinert, mitochondrial myopathies like disturbances of the catabolic metabolism in the muscle, carbohydrate and lipid storage myopathies, glycogenoses, myoglobinuria, malignant hyperthermia, polymyalgia rheumatics, dermatomyositis, multiple myositis, primary myocardial disease, cardiomyopathy; disorders of the ectoderm, neurofibromatosis, scleroderma and polyar teritis, Louis-Bar syndrome, von Hippel-Lindau disease; Sturge-Weber syndrome, tuberous sclerosis, amyloidosis, porphyria; sexual dysfunction of the male and female; confused states and seizures due to inappropriate secretion of antidiuretic hormone from the pituitary gland, Liddle's syndrome, Bartter's syndrome, Fanconi's I syndrome, or renal electrolyte wasting.

The disease, disorder, or condition may be or include a transplant rejection related condition, such as acute and chronic allograft rejection following solid organ transplant, for example, transplantation of kidney, heart, liver, lung, and cornea, chronic graft versus host disease, skin graft rejection, and bone marrow transplant rejection, or immunosuppression.

The disease, disorder, or condition may be or include a genitourinary related condition, such as nephritis (interstitial, acute interstitial (allergic), and glomerulonephritis), nephrotic syndrome, cystitis including acute and chronic (interstitial) cystitis and Hunner's ulcer, acute and chronic urethritis, prostatitis, epididymitis, oophoritis, salpingitis, vulvo vaginitis, vulvovaginal candidiasis, Peyronie's disease, and erectile dysfunction, renal disease, renal fibrosis, nephropyelitis, secondary contracted kidney, steroid dependent and steroid-resistant nephrosis, or Goodpasture's Syndrome.

The disease, disorder, or condition may be or include a CNS related disease or disorder, such as neurodegenerative diseases, Alzheimer's disease and other cementing disorders including CJD and nvCJD, amyloidosis, and other demyelinating syndromes, cerebral atherosclerosis and vasculitis, temporal arteritis, myasthenia gravis, acute and chronic so pain (acute, intermittent or persistent, whether of central or peripheral origin) including post-operative, visceral pain, headache, migraine, neuralgia (including trigeminal), atypical facial pain, joint and bone pain, pain arising from cancer and tumor invasion, neuropathic pain syndromes including diabetic, post-herpetic, and HIV-associated neuropathies, neurosarcoidosis, to brain injuries, cerebrovascular diseases and their consequences, Parkinson's disease, corticobasal degeneration, motor neuron disease, dementia, including ALS (Amyotrophic-lateral sclerosis), multiple sclerosis, traumatic brain injury, stroke, post-stroke, post-traumatic brain injury, and small-vessel cerebrovascular disease, dementias, vascular dementia, dementia with Lewy bodies, frontotemporal dementia and Parkinsonism linked 1 to chromosome 17, frontotemporal dementias, including Pick's disease, is progressive supranuclear palsy, corticobasal degeneration, Huntington's disease, thalamic degeneration, HIV dementia, schizophrenia with dementia, and Korsakoffs psychosis, within the meaning of the definition are also considered to be CNS disorders central and peripheral nervous system complications of malignant, infectious or autoimmune processes, algesia, cerebral infarction, attack, cerebral ischemia, head injury, spinal cord injury, myelopathic muscular atrophy, Shy-Drager syndrome, Reye's syndrome, progressive multifocal leukoencephalopathy, normal pressure hydrocephalus, sclerosing panencephalitis, frontal lobe type dementia, acute anterior poliomyelitis (poliomyelitis), poliomyelitis neurosis, viral encephalitis, allergic encephalomyelitis, epileptic encephalopathies, Creutzfeldt-Jakob disease, Kuru disease, bovine spongiform encephalopathy (mad cow disease), scrapie, epilepsy, cerebral amyloid angiopathy, depression, mania, manic-depressive psychosis, hereditary cerebellar ataxia, peripheral neuropathy, Nasu-Hakola syndrome, or Machado-Joseph disease.

The disease, disorder, or condition may be or include an inflammatory or immunological disease or disorder, such as general inflammation (of the ocular, nasal, pulmonary, and gastrointestinal passages), mastocytosis/mast cell disorders (cutaneous, systemic, mast cell activation syndrome, and pediatric mast cell diseases), mastitis (mammary gland), vaginitis, vasculitis (e.g., necrotizing, cutaneous, and hypersensitivity vasculitis), Wegener granulamatosis, myositis (including polymyositis, dermatomyositis), basophil related diseases including basophilic leukemia and basophilic leukocytosis, and eosinophil related diseases such as Churg-Strauss syndrome, eosinophilic granuloma, lupus erythematosus (such as, systemic lupus erythematosus, subacute cutaneous lupus erythematosus, and discoid lupus erythematosus), chronic thyroiditis, Hashimoto's thyroiditis, Grave's disease, type I diabetes, complications arising from diabetes mellitus, other immune disorders, eosinophilia fasciitis, hyper IgE syndrome, Addison's disease, antiphospholipid syndrome, immunodeficiency disease, acquired immune deficiency syndrome (AIDS), leprosy, Sezary syndrome, paraneoplastic syndromes, and other autoimmune disorders, fervescence, myositis, nervous diseases selected from multiple myositis, bursitis, Evans syndrome, leukotriene B4-mediated diseases, idiopathic hypoparathyroidism, nephrotic syndrome lupus, or immunosuppression.

The disease, disorder, or condition may be or include a cardiovascular disease or disorder, such as congestive heart failure, myocardial infarction; ischemic diseases of the heart, all kinds of atrial and ventricular arrhythmias, hypertension, cerebral trauma, occlusive vascular disease, stroke, cerebrovascular disorder, atherosclerosis, restenosis, affecting the coronary and peripheral is circulation, pericarditis, myocarditis, inflammatory and auto-immune cardiomyopathies including myocardial sarcoid, endocarditis, valvulitis, and aortitis including infective (e.g. syphilitic), hypertensive vascular diseases, peripheral vascular diseases, and atherosclerosis, vasculitides, disorders of the proximal and peripheral veins including phlebitis and thrombosis, including deep vein thrombosis and complications of varicose veins, aortic aneurism, periarteritis nodosa, cardiac fibrosis, post-myocardial infarction, idiopathic cardiomyopathy, or angioplasty.

The disease, disorder, or condition may be or include an oncological disease or disorder, such as common cancers (prostate, breast, lung, ovarian, pancreatic, bowel and colon, abdomen, stomach (and any other digestive system cancers), liver, pancreas, peritoneum, endocrine glands (adrenal, parathyroid, pituitary, testicles, ovary, thymus, thyroid), eye, head, neck, nervous system (central and peripheral), lymphatic system, blood, pelvic, skin, bone, soft tissue, spleen, thoracic, urogenital, and brain tumors), breast cancer, genitourinary cancer, lung cancer, gastrointestinal cancer, epidermoid cancer, melanoma, ovarian cancer, pancreas cancer, neuroblastoma, malignancies affecting the bone marrow (including the leukaemias) and lymphoproliferative systems, such as Hodgkin's and non-Hodgkin's lymphoma, B-cell lymphoma, follicular lymphoma, metastatic disease and tumor recurrences, and paraneoplastic syndromes, as well as hypergammaglobulinemia, lymphoproliferative diseases, disorders, and/or conditions, paraproteinemias, purpura (including idiopathic thrombocytopenic purpura), Waldenstron's Macroglobulinemia, Gaucher's Disease, histiocytosis, retinoblastoma and any other hyperproliferative disease, sarcomata, cachexia, tumor growth, tumor invasion, metastasis, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes e.g. acute or chronic myeloid leukaemia, or keratoleukoma.

The disease, disorder, or condition may be or include another disease or disorder, such as pain, migraine, sleep disorders, fever, sepsis, idiopathic thrombocytopenia pupura, post-operative adhesions, flushing, ischemic/reperfusion injury in the heart, brain, peripheral limbs, bacterial infection, viral infection, fungal infection, thrombosis, endotoxin shock, septic shock, thermal regulation including fever, Raynaud's disease, gangrene, diseases requiring anti-coagulation therapy, congestive heart failure, mucus secretion disorders, pulmonary hypotension, prostanoid-induced smooth muscle contract associated with dysmenorrhea and premature labor, premature delivery, reperfusion injury, burn, thermal injury, hemorrhage or traumatic shock, menstrual pain, menstrual cramp, dysmenorrhea, periodontosis, rickettsial infectious disease, protozoal disease, reproduction disease, toothache, pain after tooth extraction, Herpes zoster, Herpes simplex, retroperitoneal fibrosis, or various radiation injuries.

In certain embodiments, the disease is selected from the group consisting of an inflammatory disease, an autoimmune disease, an allergic disorder, and an ocular disorder. In certain embodiments, the disease is selected from the group consisting of pruritus, eczema, asthma, rhinitis, dry eye, ocular inflammation, allergic conjunctivitis, vernal conjunctivitis, vernal keratoconjunctivitis, giant papillary conjunctivitis, fungal keratitis and uveitis.

The method may include modulating the activity of one or more kinases in a subject, such as any of the kinase described above. The method may include inhibiting a kinase. The method may include activating, e.g., stimulating or enhancing the activity of, a kinase. The method may include modulating activity of a single kinase or preferentially modulating activity of a specific kinase over others. The method may include modulating activity of multiple kinases or preferentially modulating activity of two more specific kinases over others.

The method may include providing a compound of the invention. The method may include providing multiple compounds of the invention.

The method may include contacting cells containing a kinase with one or more compounds of the invention. For example and without limitation, contacting a cell with a compound may include exposing a cell to a compound, e.g., in a formulation, such as any of those described above; delivering a compound inside a cell; providing a compound to a subject and allowing a cell in the subject to become exposed to the compound. Contacting may be performed in vivo or in vitro. In vitro contact may include exposure of cells or tissue isolated from a subject. The method may include contacting cells with a single compound of the invention. The method may include contact cells with multiple compounds of the invention.

The method may include administration of a composition to a subject. The compositions may be provided by any suitable route of administration. For example and without limitation, the compositions may be administered buccally, by injection, dermally, enterally, intraarterially, intravenously, intranasally; e.g., by inhalation, intraocularly, orally, parenterally, pulmonarily, rectally, subcutaneously, systemically, topically, e.g., to the skin or eye, transdermally, or with or on an implantable medical device (e.g., stent or drug-eluting stent or balloon equivalents).

The method may include using a composition of the invention to diagnose a disease, disorder, or condition in a subject. For example, a radiolabeled form of a compound may be used a tracer in positron emission tomography (PET) to identify anatomical locations of aberrant kinase activity. PET is known in the art and described in, for example, Wadsak Wolfgang, Mitterhauser Markus (2010), “Basics and principles of radiopharmaceuticals for PET/CT”; European Journal of Radiology, 73 (3): 461-469. doi:10.1016/j.ejrad.2009.12.022; Bailey, D. L; D. W. Townsend; P. E, Valk; M. N. Maisey (2005), Positron Emission Tomography: Basic Sciences. Secaucus, NJ: Springer-Verlag, ISBN 1-85233-798-2; and Carlson, Neil (Jan. 22, 2012). Physiology of Behavior. Methods and Strategies of Research, 11th edition, Pearson, p. 151, ISBN 0205239390, the contents of each of which are incorporated herein by reference. The invention may include administering one or more compositions of the invention for both diagnostic and therapeutic purposes.

### EXAMPLES

### Example 1: Synthesis of N-(8-(methylamino)-5-phenyl-2,7-naphthyridin-3-yl)cyclopropanecarboxamide

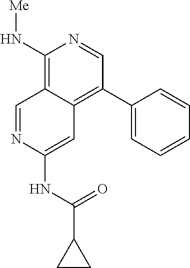 [SMILES: CNc1ncc(-c2ccccc2)c2cc(NC(=O)C3CC3)ncc12]

### Step 1: 4-bromo-6-chloro-2,7-naphthyridin-1(2H)-one

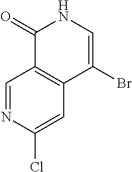 [SMILES: O=c1[nH]cc(Br)c2cc(Cl)ncc12]

To a solution of 6-chloro-2,7-naphthyridin-1-ol (1 g; 5.53 mmol; 1.00 eq.) in DMF (15 mL) under nitrogen was added NBS (1 g; 5.61 mmol; 1.00 eq.) at 0° C. and the reaction mixture was stirred at room temperature for 3 hours. The progress of the reaction was monitored via LCMS. The precipitated solids were collected by filtration and washed with water and then dried under vacuum to afford 4-bromo-6-chloro-2,7-naphthyridin-1(2H)-one (1.16 g, 81%) as a white solid. LCMS (ESI) m/z 258.9, [M+H]⁺.

### Step 2: 4-bromo-6-chloro-2-((2-trimethylsilylethoxy)methyl)-2,7-naphthyridin-1(2H)one

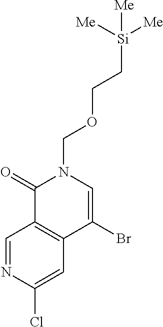 [SMILES: C[Si](C)(C)CCOCn1cc(Br)c2cc(Cl)ncc2c1=O]

To a mixture of 4-bromo-6-chloro-2,7-naphthyridin-1(2H)-one (1.16 g; 4.47 mmol; 1.00 eq.) and Cs₂CO₃ (2.9 g; 8.89 mmol; 2.00 eq.) in THF (20 mL) under nitrogen was added TBAT (166 mg; 0.449 mmol; 0.10 eq.) at 0° C. To this reaction mixture was added (2-(chloromethoxy)ethyl)-trimethylsilane (1.31 g; 7.89 mmol; 1.76 eq.) and the reaction mixture was stirred at room temperature for 1 hour. The desired product was observed via LCMS. The precipitated solids were collected by filtration and washed with THF (20 mL) and dried under vacuum to afford 4-bromo-6-chloro-2-((2-(trimethylsilyl)ethoxy)methyl)-2,7-naphthyridin-1(2H)-one as a white solid (1.08 g, 62%). LCMS (ESI) m/z 389.0, [M+H]⁺.

### Step 3: 6-chloro-4-phenyl-2-((2-(trimethylsilyl)ethoxy)methyl)-2,7-naphthyridin-1(2H)-one

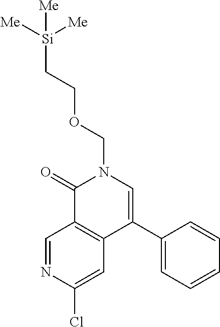 [SMILES: C[Si](C)(C)CCOCn1cc(-c2ccccc2)c2cc(Cl)ncc2c1=O]

To a stirring mixture of 4-bromo-6-chloro-2-((2-(trimethylsilyl)ethoxy)methyl)-2,7-naphthyridin-1(2H)-one (160 mg; 0.411 mmol; 1.00 eq.) and phenyl boronic acid (40 mg; 0.327 mmol; 0.80 eq.), Pd(PPh₃)₄ (47.6 mg; 0.041 mmol; 0.10 eq.) in DME/water (5:1, 6 mL) was added Na₂CO₃ (87.4 mg; 0.824 mmol; 2.00 eq.) at room temperature and the reaction mixture was stirred under nitrogen at 100° C. for 3 hours. The desired product was observed via LCMS. The reaction was concentrated under vacuum. The residue was purified by flash chromatography on silica gel column using EtOAc in petroleum ether (10-30%) as eluent to provide 6-chloro-4-phenyl-2-((2-(trimethylsilyl)ethoxy)methyl)-2,7-naphthyridin-1(2H)-one as a yellow solid (78 mg, 49%). LCMS (ESI) m/z 387.1, [M+H]⁺.

### Step 4: N-(8-oxo-5-phenyl-7-((2-trimethylsilyl)ethoxy)methyl)-7,8-dihydro-2,7-naphthyridin-3-yl)cyclopropanecarboxamide

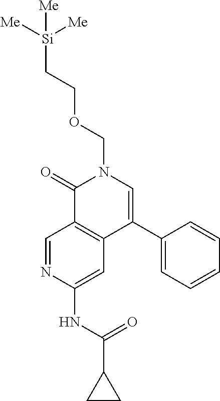 [SMILES: C[Si](C)(C)CCOCn1cc(-c2ccccc2)c2cc(NC(=O)C3CC3)ncc2c1=O]

A mixture of 6-chloro-4-phenyl-2-((2-(trimethylsilylethoxy)methyl)-2,7-naphthyridin-1(2H)-one (58 mg; 0.150 mmol; 1.00 eq.), Pd₂(dba)₃ (13.8 mg; 0.015 mmol; 0.10 eq.), XantPhos (17.4 mg; 0.030 mmol; 0.20 eq.), Cs₂CO₃ (98.0 mg 0.301 mmol; 2.00 eq.) and cyclopropane-carboxamide (38.3 mg; 0.450 mmol; 3.00 eq.) in dioxane (3 mL) was stirred at 110° C. for 2 hours. The mixture was allowed to cool down to room temperature and then concentrated under vacuum. The residue was purified via a silica gel column using EtOAc in petroleum ether (10-30%) as eluent to provide N-(8-oxo-5-phenyl-7-((2-(trimethylsilyl)ethoxy)methyl)-7,8-dihydro-2,7-naphthyridin-3-yl)cyclopropanecarboxamide as a white solid (60 mg, 92%). LCMS (ESI) m/z 436.2, [M+H]⁺.

### Step 5: N-(8-oxo-5-phenyl-7,8-dihydro-: 1-naphthyridin-3-yl)cyclopropanecarboxamide

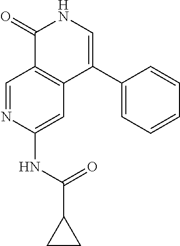 [SMILES: O=C(Nc1cc2c(-c3ccccc3)c[nH]c(=O)c2cn1)C1CC1]

To a solution of N-(8-oxo-5-phenyl-7-((2-(trimethylsilyl)ethoxy)methyl)-7,8-dihydro-2,7-naphthyridin-3-yl)cyclopropanecarboxamide (80 mg; 0.183 mmol; 1.00 eq.) in MeOH (0.5 mL) was added a solution of HCl in dioxane (4 M, 2 mL). The reaction mixture was stirred under nitrogen at room temperature for 1 hour. The desired product was observed via LCMS. The mixture was concentrated under vacuum to afford N-(8-oxo-5-phenyl-7,8-dihydro-2,7-naphthyridin-3-yl)cyclopropanecarboxamide as a yellow solid (50 mg, 89%). LCMS (ESI) m/z 306.1, [M+H]⁺.

### Step 6: N-(8-chloro-5-phenyl-2,7-naphthyridin-3-yl)cyclopropanecarboxamide

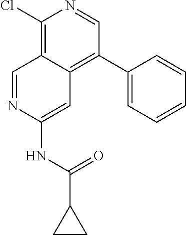 [SMILES: O=C(Nc1cc2c(-c3ccccc3)cnc(Cl)c2cn1)C1CC1]

N-(8-oxo-5-phenyl-7,8-dihydro-2,7-naphthyridin-3-yl)cyclopropanecarboxamide (50 mg; 0.162 mmol; 1.00 eq.) was dissolved in POCl₃ (2 mL). The resulting mixture was stirred at 100° C. for 1 h. The desired product was observed via LCMS. The solvent was concentrated under vacuum to afford N-(8-chloro-5-phenyl-2,7-naphthyridin-3-yl)cyclopropanecarboxamide as a yellow solid (35 mg, 66%), LCMS (ESI) m/z 324.1, [M+H]⁺.

### Step 7: N-(8-(methylamino)-5-phenyl-2,7-naphthyridin-3-yl)cyclopropanecarboxamide

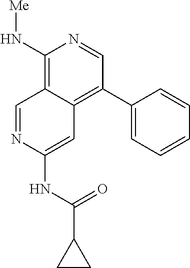 [SMILES: CNc1ncc(-c2ccccc2)c2cc(NC(=O)C3CC3)ncc12]

N-(8-chloro-5-phenyl-2,7-naphthyridin-3-yl)cyclopropanecarboxamide (35 mg; 0.108 mmol; 1.00 eq.) was dissolved in a solution of methylamine in THF (2M, 2 mL) and the reaction mixture was stirred at 60° C. under nitrogen for 12 hours. Upon completion, the mixture was concentrated under vacuum. The residue was purified by flash chromatography on pre-packed C18 column using 20-50% of MeCN in water (10 mmol/L NH₄HCO₃) as eluent to provide N-(8-(methylamino)-5-phenyl-2,7-naphthyridin-3-yl)cyclopropanecarboxamide as a white solid (5.2 mg, 15%). LCMS (ESI) m/z 319.1, [M+H]⁺. ¹H NMR (400 MHz, DMSO-d₆) δ 10.92 (s, 1H), 9.39 (s, 1H), 8.35 (s, 1H), 8.02-7.90 (m, 2H), 7.55-7.30 (m, 5H), 3.02 (d, J=4.4 Hz, 3H), 2.07-1.95 (m, 1H), 0.84-0.6 (m, 4H)

### Example 2: Synthesis of N-(8-(methylamino)-2,7-naphthyridin-3-yl)cyclopropanecarboxamide

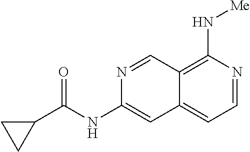 [SMILES: CNc1nccc2cc(NC(=O)C3CC3)ncc12]

### Step 1: 6-chloro-4-methylnicotinamide

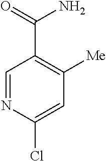 [SMILES: Cc1cc(Cl)ncc1C(N)=O]

To a stirred solution of 6-chloro-4-methylnicotinic acid (50.0 g; 0.292 mol; 1.00 eq.) in CH₂Cl₂(1. L) was added HATU (170 g; 0.447 mol; 1.50 eq.). The reaction was stirred for 20 min at room temperature. To this mixture was added NH₄Cl (155 g; 2.92 mol; 10.0 eq.) and DIPEA (113 g; 0.875 mol; 3.00 eq.) and stirred for 2 h at room temperature under nitrogen atmosphere. The desired product was observed via LCMS. The resulting mixture was diluted with water (1 L) and extracted with CH₂Cl₂ (200 mL×5). The organic lavers were washed with a sat. NaCl solution, dried over anhydrous Na₂SO₄. After filtration, the filtrate was concentrated under reduced pressure. The residue was purified by flash chromatography on silica gel column using EtOAc in petroleum ether (25-60%) as eluent to provide 6-chloro-4-methylnicotinamide as a brown solid (33 g, 66%). LCMS (ESI) m/z 171.0, [M+H]⁺.

### Step 2: (Z)-6-chloro-N-((dimethylamino)methylene)-4-methylnicotinamide

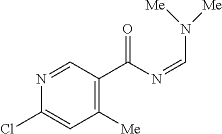 [SMILES: Cc1cc(Cl)ncc1C(=O)/N=C\N(C)C]

To a stirring mixture of 6-chloro-4-methylnicotinamide (51 g; 300 mmol; 1.00 eq.) in methyltetrahydrofuran (300 mL) was added DMF-DMA (53.5 g; 449 mmol 1.50 eq.). The resulting mixture was stirred for 1 hour at 80° C. under nitrogen atmosphere. The desired product was observed via LCMS. The mixture was cooled and concentrated under reduced pressure to afford (Z)-6-chloro-N-((dimethylamino)methylene)-4-methylnicotinamide as a brown solid (65.0 g, crude). The crude product was used in the next step directly without further purification. LCMS (ESI) m/z 226.1, [M+H]⁺.

### Step 3: 6-chloro-2,7-naphthyridin-1(2H)-one

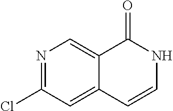 [SMILES: O=c1[nH]ccc2cc(Cl)ncc12]

To a solution of (Z)-6-chloro-N-((dimethylamino)methylene)-4-methylnicotinamide (60.0 g; 266 mmol; 1.00 eq.) in THF (387 mL) was added t-BuOK (1.0 Min THF, 400 mL) slowly at 0° C. and the reaction mixture was stirred for 0.5 h at 80° C. under nitrogen atmosphere. The desired product was detected via LCMS. The precipitated solids were collected by filtration and washed with THF (2×20 mL) to afford the crude product. The crude product was dissolved with water (500 mL), and acidified to pH=6 with a solution of HCl (1 M) and the precipitate solids were collected by filtration and washed with water. The solids were dried in vacuum to afford 6-chloro-2,7-naphthyridin-1(2H)-one as a brown solid (40 g, 83%). LCMS (ESI) m/z 181.0, [M+H]⁺. ¹H NMR (400 MHz, DMSO-d₆) δ 11.69 (s, 1H), 9.09 (s, 1H), 7.76 (s, 1H), 7.52-7.40 (m, 1H), 6.53 (d, J=7.2 Hz, 1H).

### Step 4: N-(8-hydroxy-2,7-naphthyridin-3-yl)cyclopropanecarboxamide

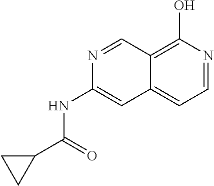 [SMILES: O=C(Nc1cc2ccnc(O)c2cn1)C1CC1]

To a stirring mixture of 6-chloro-2,7-naphthyridin-1(2H)-one (8.5 g, 47.1 mmol, 1.00 eq.) in 1,4-dioxane (500 mL) were added Pd₂(dba)³ (4.25 g, 4.64 mmol, 0.10 eq.), XantPhos (5.44 g, 9.40 mmol, 0.20 eq.), cyclopropanecarboxamide (162 g, 190 mmol, 4.00 eq.) and Cs₂CO₃ (30.6 g, 93.9 mmol, 2.00 eq.) under nitrogen atmosphere. The reaction was stirred at 110° C. for 3 h. The desired product was observed via LCMS. The reaction was concentrated in vacuo, the residue was purified by flash chromatography on silica gel column using EtOAc/petroleum ether (30-100%) and MeOH/CH₂Cl₂ (2-20%) as eluent to provide 8.3 g of the crude product. The crude was dissolved with a mixture solvent of CH₂Cl₂/EtOAc (2:1, 450 mL) and allowed to stir overnight, then filtered, the filter cake was washed with CH₂Cl₂ (200 mL) and dried over to afford N-(8-hydroxy-2,7-naphthyridin-3-yl)cyclopropanecarboxamide as a yellow solid (6.5 g, 60%). LCMS (ESI) m/z 230.1, [M+H]⁺.

### Step 5: N-(8-chloro-2,7-naphthyridin-3-yl)cyclopropanecarboxamide

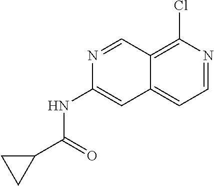 [SMILES: O=C(Nc1cc2ccnc(Cl)c2cn1)C1CC1]

N-(8-hydroxy-2,7-naphthyridin-3-yl)cyclopropanecarboxamide (4.4 g, 19.2 mmol, 1.00 eq.) was dissolved in POCl₃ (55 mL). The reaction was stirred at 100° C. for 30 min. The reaction was cooled down and then concentrated in vacuo. The residue was diluted with CH₂Cl₂ (300 mL) and neutralized to pH 7 with a saturated NaHCO₃ solution. The organic phase was concentrated in vacuo. The residue was purified by flash chromatography on silica gel column using EtOAc/CH₂Cl₂ (20-50%) to give N-(8-cloro-2,7-naphthyridin-3-yl)cyclopropanecarboxamide as off-white solid (1.85 g, 39%). LCMS (ESI) m/z 248.1, [M+H]⁺.

### Step 6: N-(8-(methylamino)-2,7-naphthyridin-3-yl)cyclopropanecarboxamide

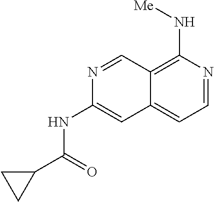 [SMILES: CNc1nccc2cc(NC(=O)C3CC3)ncc12]

To a stirring mixture of N-(8-chloro-2,7-naphthyridin-3-yl)cyclopropanecarboxamide (920 mg, 3.71 mmol, 1.00 eq.) in NMP (10 mL) were added methanamine hydrochloride (1.0 g; 14.8 mmol; 4.00 eq.) and DIPEA (2.4 g, 18.6 mmol, 5.00 eq.) under N₂. The reaction was stirred at 100° C. for 18 h. The desired product was observed via LCMS. The mixture was diluted with EtOAc (100 mL) and washed with brine (25 mL 5). The organic layer was concentrated under vacuum and purified by flash chromatography on pre-packed C18 column using 10-40% of MeCN in water (10 mmol/L NH₄HCO) to give N-(8-(methyl amino)-2,7-naphthyridin-3-yl)cyclopropanecarboxamide as a off-white solid (850 mg, 94%). 10 mg of the product was further purified by flash chromatography on pre-packed C18 column using 20%-60% of MeCN in water (10 M NH₄HCO₃) to provide N-(8-(methyl amino)-2,7-naphthyridin-3-yl)cyclopropanecarboxamide as a white solid (4.5 mg, 45%). LCMS (ESI) m/z 243.1, [M+H]⁺. ¹H NMR (400 MHz, DMSO-d₆) δ 10.91 (s, 1H), 9.30 (s, 1H), 8.16 (s, 1H), 7.95 (d, J=6.0 HZ, 1H), 7.87-7.80 (m, 1H), 6.74 (d, J=6.0 Hz, 1H), 2.96 (d, J=4.4 Hz, 3H), 2.09-2.00 (m, 1H), 0.88-0.78 (m, 4H)

### Example 3

Synthesis of N-(5-bromo-8-(methylamino)-2,7-naphthyridin-3-yl)cyclopropanecarboxamide

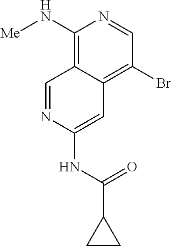 [SMILES: CNc1ncc(Br)c2cc(NC(=O)C3CC3)ncc12]

To a stirring mixture of N-(8-(methylamino)-2,7-naphthyridin-3-yl)cyclopropanecarboxamide (Example 2, step 6) (1.9 g, 7.84 mmol, 1.00 eq.) in DMF (12 mL) at 0° C. under nitrogen atmosphere, NBS (1.40 g, 7.84 mmol, 1.00 eq.) was added at 0° C. Then the reaction was warmed to room temperature for 1 h. The desired product was observed via LCMS. The resulting mixture was diluted with EtOAc (150 mL) and washed with brine (50 mL×5). The organic layer was dried; concentrated under vacuum and purified by flash chromatography on silica gel column using 2-10% of MeOH in CH₂Cl₂ to afford 2.5 g of the crude product. Then the crude product was purified by reverse phase preparative HPLC (Prep-C18, 5 μM OBD column, 19×250 mm, water; gradient elution of 40-50% MeCN in water over a 8 min period, where both water and MeCN contain 10 mmol/L NH₄HCO₃, flow rate: 60 mL/min, detector UV wavelength: 254 nm) to give N-(5-bromo-8-(methylamino)-2,7-naphthyridin-3-yl)cyclopropanecarboxamide as a white solid (327 mg, 13%). LCMS (ESI) m/z 321.0, [M+H]⁺. ¹H NMR (400 MHz, DMSO-d₆) δ 11.13 (s, 1H), 9.32 (s, 1H), 8.44 (s, 1H), 8.16 (s, 1H), 8.14-8.09 (m, 1H), 2.95 (d, J=4.4 Hz, 3H), 2.12-2.04 (m, 1H), 0.92-0.82 (m, 4H).

### Example 4: Synthesis of N-(8-(methylamino-5-(pyridin-2-yl)-2,7-naphthyridin-3-yl)cyclopropanecarboxamide

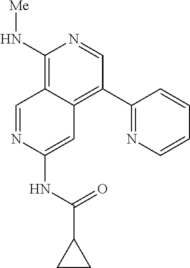 [SMILES: CNc1ncc(-c2ccccn2)c2cc(NC(=O)C3CC3)ncc12]

To a solution of N-(5-bromo-8-(methylamino)-2,7-naphthyridin-3-yl)cyclopropanecarboxamide (35.0 mg; 0.109 mmol; 1.00 eq.) in 1,4-dioxane (1.5 mL) were added 2-(tributylstannyl)pyridine (80.2 mg; 0.218 mmol; 2.00 eq.), CuI (4.1 mg; 0.022 mmol; 0.20 eq.), Pd(PPh₃)₄ (25.2 mg; 0.022 mmol; 0.20 eq.) and LiCl (116 mg; 0.273 mmol; 2.50 eq.). The reaction mixture was stirred for 3 h at 110° C. under nitrogen atmosphere. The resulting mixture was diluted with EtOAc (50 mL). The organic layers were washed with brine (5×3 mL), dried over anhydrous Na₂SO₄. After filtration, the filtrate was concentrated under reduced pressure. The residue was purified by flash chromatography on pre-packed C18 column using 20-50% of MeCN in water (10 mmol/L NH₄HCO₃) as eluent to provide N-(8-(methylamino)-5-(pyridin-2-yl)-2,7-naphthyridin-3-yl)cyclopropanecarboxamide as a white solid (4.6 mg, 13%). LCMS (ESI) m/z 320.1, [M+H]⁺, ¹H NMR (400 MHz, DMSO-d₆) δ 10.94 (s, 1H), 9.40 (s, 1H), 8.72 (s, 1H), 8.70-8.66 (m, 1H), 8.27-8.19 (m, 1H), 8.15 (s, 1H), 7.93-7.87 (m, 1H), 7.61-7.57 (m, 1H), 7.39-7.33 (m, 1H), 3.04 (d, J=4.4 Hz, 3H), 2.06-1.99 (m, 1H), 0.82-0.78 (m, 4H).

### Example 5: Synthesis of N-(5-(3-methoxypyridin-2-yl)-8-(methylamino)-2,7-naphthyridin-3-yl)cyclopropanecarboxamide

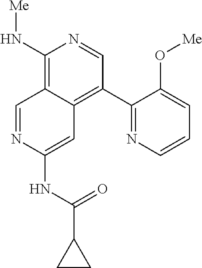 [SMILES: CNc1ncc(-c2ncccc2OC)c2cc(NC(=O)C3CC3)ncc12]

### Step 1: 3-methoxy-2-(tributylstannyl)pyridine

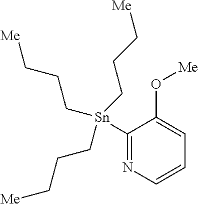 [SMILES: CCC[CH2][Sn]([CH2]CCC)([CH2]CCC)[c]1ncccc1OC]

To a solution of 2-chromo-3-methoxypyridine 500 mg; 2.65 mmol; 1.00 eq.) in 1,4-dioxane (5 mL) were added Pd₂(dba)₃ (244.8 mg; 0.267 mmol; 0.10 eq.), tricyclohexylphosphine (167.7 mg; 0.598 mmol; 0.22 eq.), LiCl (566.8 mg; 13.3 mmol; 5.00 eq.). To this mixture was added 1,1,1,2,2,2-hexabutyldistannane (2.32 g; 4.01 mmol; 1.50 eq.). The reaction was stirred at 110° C. for 1 h. The mixture was allowed to cool down to room temperature. The desired product was detected via LCMS. The resulting mixture was diluted with CH₂Cl₂. (100 mL) and washed with a saturated NaCl solution (3×5 mL). The organic layer was dried with Na₂SO₄, filtered and the solvent was removed under reduced pressure. The residue was purified by flash chromatography on neutral Al₂O₃ column using 1-10% of EtOAc in petroleum ether as eluent to provide 3-methoxy-2-(tributylstannyl)pyridine as a yellow oil (1.0 g, 93%). LCMS (ESI) m/z 400.2, [M+H]⁺.

### Step 2: N-(5-(3-methoxypyridin-2-yl)-8-(methylamino)-2,7-naphthyridin-3-yl)cyclopropane carboxamide

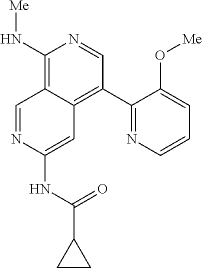 [SMILES: CNc1ncc(-c2ncccc2OC)c2cc(NC(=O)C3CC3)ncc12]

To a stirring mixture of N-(5-bromo-8-(methylamino)-2,7-naphthyridin-3-yl)cyclopropanecarboxamide (50 mg; 0.16 mmol; 1.00 eq.) in 1,4-dioxane (2 mL) were added 3-methoxy-2-(tributylstannyl)pyridine (312 mg; 0.78 mmol; 5.00 eq.), CuI (6 mg; 0.03 mmol; 0.20 eq.), Pd(PPh₃)₄ (36 m g; 0.03 mmol; 0.20 eq.), and LiCl (16 mg; 0.37 mmol; 2.50 eq.). The reaction was stirred for 5 h at 110° C. under nitrogen atmosphere. The resulting mixture was diluted with EtOAc (60 mL) an d washed with a sat. NaCl solution (2×5 mL), dried over anhydrous Na₂SO₄. After filtration, the organic layers filtrate was concentrated under reduced pressure. The residue was purified by a silica gel column using 2-10% of MeOH, in CH₂Cl₂ as eluent to provide the crude product. The crude product was purified by flash chromatography on pre-packed C18 column using 20-60% of MeCN in water (10 mmol/L NH₄HCO₃) to provide N-(5-(3-methoxypyridin-2-yl)-8-(methylamino)-2,7-naphthyridin-3-yl)cyclopropanecarboxamide as a white solid (22.8 mg, 41%). LCMS (ESI) m/z 350.2, [M+H]⁺; ¹H NMR (400 MHz, DMSO-d₆) δ 10.84 (s, 1H), 9.35 (s, 1H), 8.27-8.23 (m, 1H), 8.03-7.97 (m, 3H), 7.60-7.55 (m, 1H), 7.43-7.37 (m, 1H), 3.71 (s, 3H), 3.02 (d, J=4.4 Hz, 3H), 2.03-1.95 (m, 1H), 0.79-0.71 (m, 4H).

### Example 6: Synthesis of N-(5-(1-methyl-1H-pyrazol-5-yl)-8-(methylamino)-2,7-naphthyridin-3-yl)cyclopropanecarboxamide

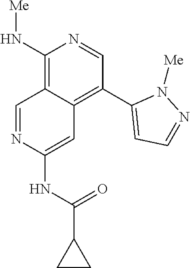 [SMILES: CNc1ncc(-c2ccnn2C)c2cc(NC(=O)C3CC3)ncc12]

A solution of N-(5-bromo-8-(methylamino)-2,7-naphthyridin-3-yl)cyclopropanecarboxamide (20 mg; 0.062 mmol; 1.00 eq.) in a mixture solvent of DME/water (5:1, 1.2 mL) were added (1-methyl-1H-pyrazol-5-yl)boronic acid (11.8 mg; 0.094 mmol; 1.5 eq.); Pd(PPh₃)₄(7.2 mg; 0.006 mmol; 0.10 eq.) and Na₂CO₃ (13.2 mg, 0.125 mmol; 2.00 eq.). The resulting mixture was stirred at 100° C. for 3 h under nitrogen atmosphere. The desired product was observed via LCMS. The resulting mixture was concentrated under reduced pressure. The residue was purified by flash chromatography on silica gel column using 3-10% of MeOH in CH₂Cl₂ as eluent to provide N-(5-(1-methyl-1H-pyrazol-5-yl)-8-(methylamino)-2,7-naphthyridin-3-yl)cyclopropane-carboxamide as a yellow solid (16 mg, crude). The crude product was purified by flash chromatography on pre-packed C18 column using 20-50% of MeCN in water (0.05% formic acid) as eluent to provide N-(5-(1-methyl-1H-pyrazol-5-yl)-8-(methylamino)-2,7-naphthyridin-3-yl)cyclopropanecarboxamide as a white solid (4.8 mg, 23%). LCMS (ESI) m/z 323.2, [M+H]⁺. NMR (400 MHz, DMSO-d₆) δ 10.98 (s, 1H), 9.39 (s, 1H), 8.20-8.11 (m, 1H), 8.01 (s, 1H), 7.98 (s, 1H), 7.54 (d, J=1.6 Hz, 1H), 7.30 (d, J=1.6 Hz, 1H), 3.64 (s, 3H), 2.97 (d, J=4.4 Hz, 3H), 2.10-1.98 (m, 1H), 0.90-0.78 (m, 4H).

### Examples 7-25

The Examples in Table 1 were prepared using a similar experimental procedure used to prepare Example 6 using N-(5-bromo-8-(methylamino)-2,7-naphthyridin-3-yl)cyclopropanecarboxamide (Example 3) as the common intermediate and appropriate boronic ester or acid.

TABLE 1

| Example | Structure and Name | MS and ¹H NMR |
|---|---|---|
| Example 7 | 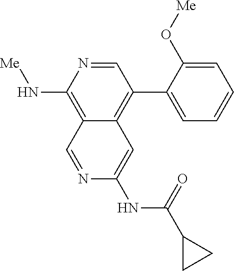 [SMILES: CNc1ncc(-c2ccccc2OC)c2cc(NC(=O)C3CC3)ncc12] | LCMS (ESI) m/z 349.2, [M + H]⁺. ¹H NMR (400 MHz, DMSO-d₆) δ 10.83 (s, 1H), 9.34 (s, 1H), 7.96-7.77 (m, 3H), 7.44-7.34 (m, 1H), 7.23-7.09 (m, 2H), 7.08-7.00 (m, 1H), 3.64 (s, 3H), 3.05- 2.96 (m, 3H), 2.06-1.93 (m, 1H), 0.82- 0.68 (m, 4H). |
| | N-(5-(2-methoxyphenyl)-8- | |
| | (methylamino)-2,7- | |
| | naphthyridin-3- | |
| | yl)cyclopropanecarboxamide | |
| Example 8 | 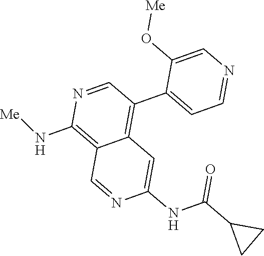 [SMILES: CNc1ncc(-c2ccncc2OC)c2cc(NC(=O)C3CC3)ncc12] | LCMS (ESI) m/z 350.2, [M + H]⁺. ¹H NMR (400 MHz, DMSO-d₆) δ 10.92 (s, 1H), 9.36 (s, 1H), 8.50 (s, 1H), 8.30 (d, J = 4.4 Hz, 1H), 8.09-8.04 (m, 1H), 7.90 (s, 2H), 7.26 (d, J = 4.4 Hz, 1H), 3.78 (s, 3H), 3.01 (d, J = 4.4 Hz, 3H), 2.06-1.96 (m, 1H), 0.79-0.74 (m, 4H). |
| | N-(5-(3-methoxypyridin-4-yl)- | |
| | 8-(methylamino)-2,7- | |
| | naphthyridin-3- | |
| | yl)cyclopropanecarboxamide | |
| Example 9 | 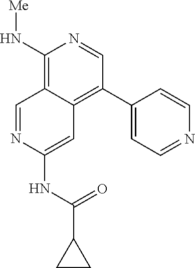 [SMILES: CNc1ncc(-c2ccncc2)c2cc(NC(=O)C3CC3)ncc12] | LCMS (ESI) m/z 320.1, [M + H]⁺. ¹H NMR (400 MHz, DMSO-d₆) δ 10.99 (s, 1H), 9.40 (s, 1H), 8.67-8.64 (m, 2H), 8.40 (s, 1H), 8.19-8.11 (m, 1H), 8.03 (s, 1H), 7.48-7.44 (m, 2H), 3.02 (d, J = 4.4 Hz, 3H), 2.07-1.98 (m, 1H), 0.82-0.78 (m, 4H). |
| | N-(8-methylamino)-5- | |
| | (pyridin-4-yl)-2,7- | |
| | naphthyridin-3- | |
| | yl)cyclopropanecarboxamide | |
| Example 10 | 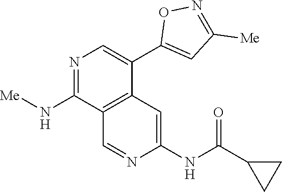 [SMILES: CNc1ncc(-c2cc(C)no2)c2cc(NC(=O)C3CC3)ncc12] | LCMS (ESI) m/z 324.1, [M + H]⁺. ¹H NMR (400 MHz, DMSO-d₆) δ 11.07 (s, 1H), 9.40 (s, 1H), 8.72 (s, 1H), 8.43- 8.39 (m, 1H), 8.39 (s, 1H), 6.57 (s, 1H), 3.03 (d, J = 4.4 Hz, 3H), 2.31 (s, 3H), 2.10-2.03 (m, 1H), 0.90-0.80 (m, 4H). |
| | N-(8-(methylamino)-5-(3- | |
| | methylisoxazol-5-yl)-2,7- | |
| | naphthyridin-3- | |
| | yl)cyclopropanecarboxamide | |
| Example 11 | 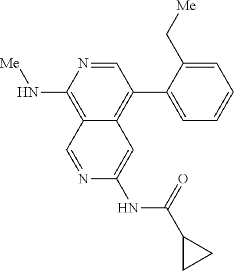 [SMILES: CCc1ccccc1-c1cnc(NC)c2cnc(NC(=O)C3CC3)cc12] | LCMS (ESI) m/z 347.2, [M + H]⁺. ¹H NMR (400 MHz, DMSO-d₆) δ 10.88 (s, 1H), 9.37 (s, 1H), 7.99-7.93 (m, 1H), 7.80 (s, 1H), 7.76 (s, 1H), 7.40-7.34 (m, 2H), 7.29-7.24 (m, 1H), 7.13-7.09 (m, 1H), 3.01 (d, J = 4.4 Hz, 3H), 2.44-2.33 (m, 1H), 2.32-2.21 (m, 1H), 2.00-1.93 (m, 1H), 0.95 (t, J = 7.2 Hz, 3H), 0.76- 0.71 (m, 4H). |
| | N-(5-(2-ethylphenyl)-8- | |
| | (methylamino)-2,7- | |
| | naphthyridin-3- | |
| | yl)cyclopropanecarboxamide | |
| Example 12 | 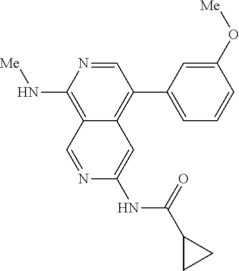 [SMILES: CNc1ncc(-c2cccc(OC)c2)c2cc(NC(=O)C3CC3)ncc12] | LCMS (ESI) m/z 349.2, [M + H]⁺. ¹H NMR (400 MHz, DMSO-d₆) δ 10.93 (s, 1H), 9.38 (s, 1H), 8.40 (s, 1H), 8.00- 7.96 (m, 1H), 7.96 (s, 1H), 7.42-7.36 (m, 1H), 7.00-6.93 (m, 3H), 3.80 (s, 3H), 3.01 (d, J = 4.4 Hz, 3H), 2.06-1.97 (m, 1H), 0.81-0.76 (m, 4H). |
| | N-(5-(3-methoxyphenyl)-8- | |
| | (methylamino)-2,7- | |
| | naphthyridin-3- | |
| | yl)cyclopropanecarboxamide | |
| Example 13 | 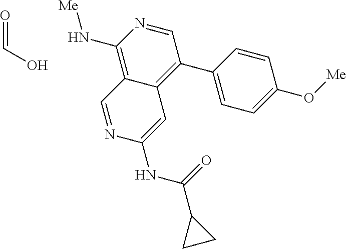 [SMILES: O=CO ; CNc1ncc(-c2ccc(OC)cc2)c2cc(NC(=O)C3CC3)ncc12] | LCMS (ESI) m/z 349.2, [M + H]⁺. ¹H NMR (400 MHz, DMSO-d₆) δ 10.91 (s, 1H), 9.37 (s, 1H), 8.32 (s, 1H), 8.16 (s, 1H), 7.96-7.87 (m, 2H), 7.35-7.29 (m, 2H), 7.08-7.02 (m, 2H), 3.83 (s, 3H), 3.01 (d, J = 4.0 Hz, 3H), 2.07-1.97 (m, 1H), 0.88-0.74 (m, 4H). |
| | N-(5-(4-methoxyphenyl)-8- | |
| | (methylamino)-2,7- | |
| | naphthyridin-3- | |
| | yl)cyclopropanecarboxamide | |
| | formate | |
| Example 14 | 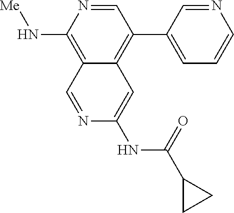 [SMILES: CNc1ncc(-c2cccnc2)c2cc(NC(=O)C3CC3)ncc12] | LCMS (ESI) m/z 320.1, [M + H]⁺. ¹H NMR (400 MHz, DMSO-d₆) δ 11.04 (s, 1H), 9.43 (s, 1H), 8.66-8.58 (m, 2H), 8.29 (s, 2H), 7.96 (s, 1H), 7.98-7.82 (m, 1H), 7.56-7.50 (m, 1H), 3.04 (d, J = 4.4 Hz, 3H), 2.06-1.98 (m, 1H), 0.82-0.76 (m, 4H). |
| | N-(8-(methylamino)-5- | |
| | (pyridin-3-yl)-2,7- | |
| | naphthyridin-3- | |
| | yl)cyclopropanecarboxamide | |
| Example 15 | 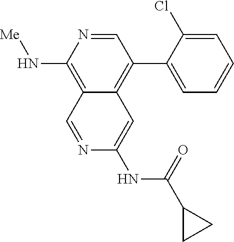 [SMILES: CNc1ncc(-c2ccccc2Cl)c2cc(NC(=O)C3CC3)ncc12] | LCMS (ESI) m/z 353.1, [M + H]⁺. ¹H NMR (400 MHz, DMSO-d₆) δ 10.90 (s, 1H), 9.38 (s, 1H), 8.08-8.00 (m, 1H), 7.85 (s, 1H), 7.83 (s, 1H), 7.66-7.57 (m, 1H), 7.52-7.42 (m, 2H), 7.42-7.35 (m, 1H), 3.02 (d, J = 4.4 Hz, 3H), 2.05-1.94 (m, 1H), 0.82-0.70 (m, 4H). |
| | N-(5-(2-chlorophenyl)-8- | |
| | (methylamino)-2,7- | |
| | naphthyridin-3- | |
| | yl)cyclopropanecarboxamide | |
| Example 16 | 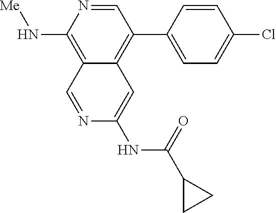 [SMILES: CNc1ncc(-c2ccc(Cl)cc2)c2cc(NC(=O)C3CC3)ncc12] | LCMS (ESI) m/z 353.1, [M + H]⁺. ¹H NMR (400 MHz, DMSO-d₆) δ 10.96 (s, 1H), 9.39 (s, 1H), 8.30 (s, 1H), 8.10- 7.88 (m, 2H), 7.55 (dd, J = 8.5, 2.2 Hz, 2H), 7.43 (dd, J = 8.5, 2.2 Hz, 2H), 3.01 (d, J = 4.4 Hz, 3H), 2.14-1.90 (m, 1H), 0.91-0.72 (m, 4H). |
| | N-(5-(4-chlorophenyl)-8- | |
| | (methylamino)-2,7- | |
| | naphthyridin-3- | |
| | yl)cyclopropanecarboxamide | |
| Example 17 | 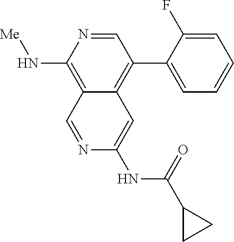 [SMILES: CNc1ncc(-c2ccccc2F)c2cc(NC(=O)C3CC3)ncc12] | LCMS (ESI) m/z 337.1, [M + H]⁺. ¹H NMR (400 MHz, DMSO-d₆) δ 10.93 (s, 1H), 9.39 (s, 1H), 8.12-8.00 (m, 2H), 7.93 (s, 1H), 7.53-7.28 (m, 4H), 3.02 (d, J = 4.4 Hz, 3H), 2.01-1.97 (m, 1H), 0.88-0.72 (m, 4H). |
| | N-(5-(2-fluorophenyl)-8- | |
| | (methylamino)-2,7- | |
| | naphthyridin-3- | |
| | yl)cyclopropanecarboxamide | |
| Example 18 | 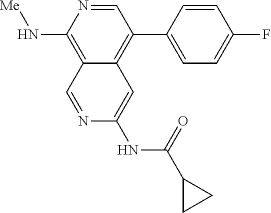 [SMILES: CNc1ncc(-c2ccc(F)cc2)c2cc(NC(=O)C3CC3)ncc12] | LCMS (ESI) m/z 337.1, [M + H]⁺. ¹H NMR (400 MHz, DMSO-d₆) δ 10.94 (s, 1H), 9.38 (s, 1H), 8.28 (s, 1H), 8.00- 7.94 (m, 1H), 7.92 (s, 1H), 7.47-7.39 (m, 2H), 7.35-7.27 (m, 2H), 3.01 (d, J = 4.4 Hz, 3H), 2.05-1.97 (m, 1H), 0.81- 0.74 (m, 4H). |
| | N-(5-(4-fluorophenyl)-8- | |
| | (methylamino)-2,7- | |
| | naphthyridin-3- | |
| | yl)cyclopropanecarboxamide | |
| Example 19 | 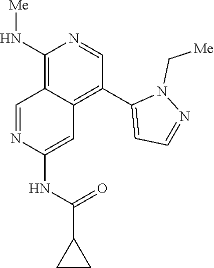 [SMILES: CCn1nccc1-c1cnc(NC)c2cnc(NC(=O)C3CC3)cc12] | LCMS (ESI) m/z 337.2, [M + H]⁺. ¹H NMR (400 MHz, Methanol-d₄) δ 9.26 (s, 1H), 7.99 (s, 1H), 7.91 (s, 1H), 7.65 (d, J = 1.6 Hz, 1H), 6.37 (d, J = 1.6 Hz, 1H), 3.98 (q, J = 7.2 Hz, 2H), 3.13 (s, 3H), 1.97-1.85 (m, 1H), 1.30 (t, J = 7.2 Hz, 3H), 1.00-0.82 (m, 4H). |
| | N-(5-(1-ethyl-1H-pyrazol-5-yl)- | |
| | 8-(methylamino)-2,7- | |
| | naphthyridin-3- | |
| | yl)cyclopropanecarboxamide | |
| Example 20 | 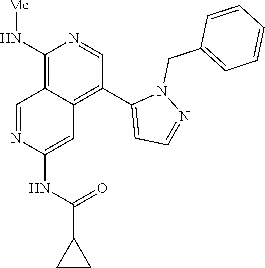 [SMILES: CNc1ncc(-c2ccnn2Cc2ccccc2)c2cc(NC(=O)C3CC3)ncc12] | LCMS (ESI) m/z 399.2, [M + H]⁺. ¹H NMR (400 MHz, DMSO-d₆) δ 10.95 (s, 1H), 9.35 (s, 1H), 8.18-8.11 (m, 1H), 7.96 (s, 1H), 7.84 (s, 1H), 7.65 (d, J = 1.6 Hz, 1H), 7.19-7.13 (m, 3H), 6.91-6.84 (m, 2H), 6.37 (d, J = 1.6 Hz, 1H), 5.11 (s, 2H), 2.98 (d, J = 4.4 Hz, 3H), 2.05- 1.97 (m, 1H), 0.84-0.75 (m, 4H). |
| | N-(5-(1-benzyl-1H-pyrazol-5- | |
| | yl)-8-(methylamino)-2,7- | |
| | naphthyridin-3- | |
| | yl)cyclopropanecarboxamide | |
| Example 21 | 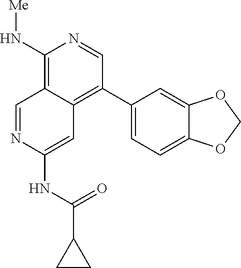 [SMILES: CNc1ncc(-c2ccc3c(c2)OCO3)c2cc(NC(=O)C3CC3)ncc12] | LCMS (ESI) m/z 363.1, [M + H]⁺. ¹H NMR (400 MHz, DMSO-d₆) δ 10.93 (s, 1H), 9.37 (s, 1H), 8.32 (s, 1H), 8.01-7.92 (m, 1H), 7.89 (s, 1H), 7.02 (d, J = 8.0 Hz, 1H), 6.95 (d, J = 1.6 Hz, 1H), 6.84 (dd, J = 8.0, 1.6 Hz, 1H), 6.09 (s, 2H), 3.04 (d, J = 4.4 Hz, 3H), 2.08-1.97 (m, 1H), 0.88- 0.75 (m, 4H). |
| | N-(5-(benzo[d][1,3]dioxol-5- | |
| | yl)-8-(methylamino)-2,7- | |
| | naphthyridin-3- | |
| | yl)cyclopropanecarboxamide | |
| Example 22 | 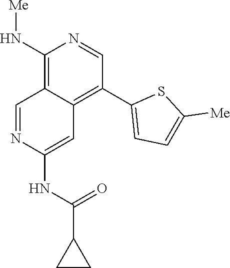 [SMILES: CNc1ncc(-c2ccc(C)s2)c2cc(NC(=O)C3CC3)ncc12] | LCMS (ESI) m/z 339.1, [M + H]⁺. ¹H NMR (400 MHz, DMSO-d₆) δ 10.99 (s, 1H), 9.37 (s, 1H), 8.61 (s, 1H), 8.09-8.00 (m, 1H), 8.02 (s, 1H), 6.95 (d, J = 3.2 Hz, 1H), 6.88 (d, J = 3.2 Hz, 1H), 3.00 (d, J = 4.4 Hz, 3H), 2.53 (s, 3H), 2.11- 2.00 (m, 1H), 0.92-0.76 (m, 4H). |
| | N-(8-(methylamino)-5-(5- | |
| | methylthiophen-2-yl)-2,7- | |
| | naphthyridin-3- | |
| | yl)cyclopropanecarboxamide | |
| Example 23 | 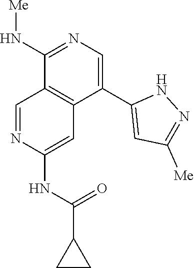 [SMILES: CNc1ncc(-c2cc(C)n[nH]2)c2cc(NC(=O)C3CC3)ncc12] | LCMS (ESI) m/z 323.2, [M + H]⁺. ¹H NMR (400 MHz, DMSO-d₆) δ 12.58 (s, 1H), 10.87 (s, 1H), 9.35 (s, 1H), 9.11- 8.43 (m, 1H), 8.20-7.83 (m, 2H), 6.23 (s, 1H), 3.00 (d, J = 4.4 Hz, 3H), 2.28 (s, 3H), 2.08-2.00 (m, 1H), 0.86-0.78 (m, 4H). |
| | N-(5-(3-methyl-1H-pyrazol-5- | |
| | yl)-8-(methylamino)-2,7- | |
| | naphthyridin-3-yl) | |
| | cyclopropanecarboxamide | |
| Example 24 | 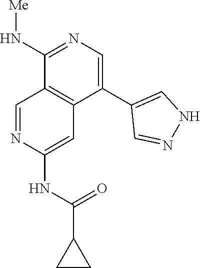 [SMILES: CNc1ncc(-c2cn[nH]c2)c2cc(NC(=O)C3CC3)ncc12] | LCMS (ESI) m/z 309.1, [M + H]⁺. ¹H NMR (400 MHz, DMSO-d₆) δ 13.00 (s, 1H), 10.97 (s, 1H), 9.36 (s, 1H), 8.53 (s, 1H), 8.02 (s, 1H), 7.97-7.65 (m, 3H), 3.10 (d, J = 4.4 Hz, 3H), 2.10-2.00 (m, 1H), 0.90-0.74 (m, 4H). |
| | N-(8-(methylamino)-5-(1H- | |
| | pyrazol-4-yl)-2,7- | |
| | naphthyridin-3- | |
| | yl)cyclopropanecarboxamide | |
| Example 25 | 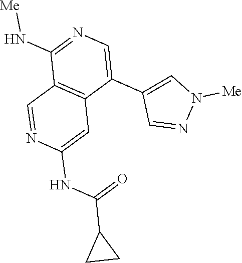 [SMILES: CNc1ncc(-c2cnn(C)c2)c2cc(NC(=O)C3CC3)ncc12] | LCMS (ESI) m/z 323.2, [M + H]⁺. ¹H NMR (400 MHz, DMSO-d₆) δ 10.95 (s, 1H), 9.35 (s, 1H), 8.48 (s, 1H), 7.97 (s, 1H), 7.89-7.87 (m, 1H), 7.86 (s, 1H), 7.59 (s, 1H), 3.91 (s, 3H), 2.98 (d, J = 4.4 Hz, 3H), 2.08-2.00 (m, 1H), 0.87-0.78 (m, 4H). |
| | N-(5-(1-methyl-1H-pyrazol-4- | |
| | yl)-8-(methylamino)-2,7- | |
| | naphthyridin-3- | |
| | yl)cyclopropanecarboxamide | |

### Example 26: Synthesis of N-(8-(methylamino)-5-(5-methylthiazol-4-yl)-2,7-naphthyridin-3-yl)cyclopropanecarboxamide

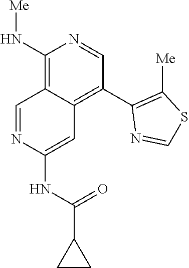 [SMILES: CNc1ncc(-c2ncsc2C)c2cc(NC(=O)C3CC3)ncc12]

To a stirring mixture of N-(8-(methylamino)-5-(4,4,5,5-tetramethyl-1,3,2-dioxaborolan-2-yl)-2,7-naphthyridin-3-yl)cyclopropanecarboxamide (Example 84, step 1) (60 mg; 0.163 mmol; 1.00 eq.) in a mixture solvent of 1,4-dioxane/water (10:1, 1.1 mL) were added 4-bromo-5-methylthiazole (43.5 mg; 0.244 mmol; 1.5 eq.), Pd(dppf)Cl₂ (11.9 mg; 0.016 mmol; 0.10 eq.) and Na₂CO₃ (34.5 mg; 0.325 mmol; 2.00 eq.). The reaction mixture was stirred for 1 h at 40° C. under nitrogen atmosphere. The reaction was monitored via LCMS. The residue was purified by flash chromatography on silica gel column using MeOH/CH₂Cl₂ (2%-10%) as eluent to afford N-(8-(methylamino)-5-(5-methylthiazol-4-yl)-2,7-naphthyridin-3-yl)cyclopropanecarboxamide (15 mg, crude). Then the crude product was purified by flash chromatography on pre-packed C18 column using 20-50% MeCN in water (10 mmol/L NH₄HCO₃) to provide N-(8-(methylamino)-5-(5-methylthiazol-4-yl)-2,7-naphthyridin-3-yl)cyclopropanecarboxamide as a white solid (4.9 mg, 9%). LCMS (ESI) m/z 340.1, [M+H]⁺. ¹H NMR (400 MHz, DMSO-d₆) δ 10.92 (s, 1.14), 9.38 (s, 1H), 9.03 (s, 1H), 8.12 (s, 1H), 8.10-8.03 (m, 1H), 7.99 (s, 1H), 3.03 (d, J=4.4 Hz, 3H), 2.35 (s, 3H), 2.10-1.98 (m, 1H), 0.88-0.72 (m, 4H).

### Example 27: Synthesis of N-(5-methyl-8-(methylamino)-2,7-naphthyridin-3-yl)cyclopropanecarboxamide

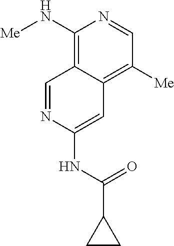 [SMILES: CNc1ncc(C)c2cc(NC(=O)C3CC3)ncc12]

To a solution of N-[5-bromo-8-(methylamino)-2,7-naphthyridin-3-yl]cyclopropanecarboxamide (40 mg; 0.125 mmol; 1.00 eq.) and K₃PO₄(53 mg; 0.250 mmol; 2.00 eq.) in dioxane/water (5:1, 4.8 mL) were added Pd(DtBPF)Cl₂ (8 mg; 0.012 mmol; 0.10 eq.) and trimethyl-1,3,5,2,4,6-trioxatriborinane (19 mg; 0.151 mmol; 1.22 eq.). After stirring for 2 h at 90° C. under a nitrogen atmosphere, the resulting mixture was concentrated under reduced pressure. The residue was purified by flash chromatography on silica gel column using MeOH in CH₂Cl₂ (2-6%) of as eluent to provide N-(8-amino-5-methyl-2,7-naphthyridin-3-yl)cyclopropanecarboxamide as a yellow solid (crude). The residue was purified by flash chromatography on pre-packed C18 column using 20%-60% of MeCN in water (10 mmol/L NH₄HCO₃) to provide N-(8-amino-5-methyl-2,7-naphthyridin-3-yl)cyclopropanecarboxamide as a white solid (15.0 mg, 46.9%), LCMS (ESI) m/z 257.1, [M+H]⁺. ¹H NMR (400 MHz, DMSO-d₆) δ 10.97 (s, 1H), 9.30 (s, 1H), 8.27 (s, 1H), 7.83 (s, 1H), 7.69-7.65 (m, 1H), 2.93 (d, J=4.4 Hz, 3H), 2.22 (s, 3H), 2.10-2.03 (m, 1H), 0.88-0.81 (m, 4H).

### Examples 28-38

The Examples in Table 2 were prepared using a similar experimental procedure used to prepare Example 27 using -[5-bromo-8-(methylamino)-2,7-naphthyridin-3-yl]cyclopropanecarboxamide as a common intermediate and appropriate boronic ester or acid or alkene,

TABLE 2

| Example | Structure and Name | MS and ¹H NMR |
|---|---|---|
| Example 28 | 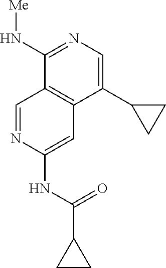 [SMILES: CNc1ncc(C2CC2)c2cc(NC(=O)C3CC3)ncc12] | LCMS (ESI) m/z 283.1, [M + H]⁺. ¹H NMR (400 MHz, DMSO-d₆) δ 10.96 (s, 1H), 9.30 (s, 1H), 8.63 (s, 1H), 7.79 (s, 1H), 7.74-7.69 (m, 1H), 2.93 (d, J = 4.4 Hz, 3H), 2.11-2.03 (m, 1H), 1.87-1.79 (m, 1H), 0.91-0.79 (m, 6H), 0.56-0.50 (m, 2H). |
| | N-(5-cyclopropyl-8- | |
| | (methylamino)-2,7- | |
| | naphthyridin-3- | |
| | yl)cyclopropanecarboxamide | |
| Example 29 | 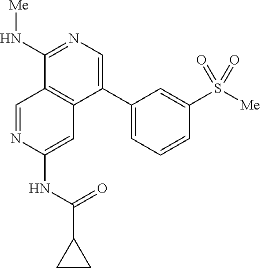 [SMILES: CNc1ncc(-c2cccc(S(C)(=O)=O)c2)c2cc(NC(=O)C3CC3)ncc12] | LCMS (ESI) m/z 397.1, [M + H]⁺. ¹H NMR (400 MHz, DMSO-d₆) δ 11.03 (s, 1H), 9.41 (s, 1H), 8.38 (s, 1H), 8.15-8.08 (s, 1H), 8.05 (s, 1H), 7.95- 7.91 (m, 2H), 7.83-7.75 (m, 2H), 3.31 (s, 3H), 3.03 (d, J = 4.4 Hz, 3H), 2.06-1.97 (m, 1H), 0.81-0.73 (m, 4H). |
| | N-(8-(methylamino)-5-(3- | |
| | (methylsulfonyl)phenyl)-2,7- | |
| | naphthyridin-3- | |
| | yl)cyclopropanecarboxamide | |
| Example 30 | 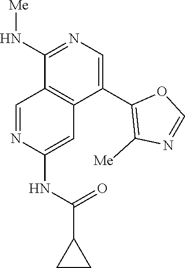 [SMILES: CNc1ncc(-c2ocnc2C)c2cc(NC(=O)C3CC3)ncc12] | LCMS (ESI) m/z 324.1, [M + H]⁺. ¹H NMR (400 MHz, DMSO-d₆) δ 11.01 (s, 1H), 9.39 (s, 1H), 8.41 (s, 1H), 8.30-8.23 (m, 1H), 8.19 (s, 1H), 8.05 (s, 1H), 3.03 (d, J = 4.4 Hz, 3H), 2.12 (s, 3H), 2.07-2.00 (m, 1H), 0.85- 0.78 (m, 4H). |
| | N-(8-(methylamino)-5-(4- | |
| | methyloxazol-5-yl)-2,7- | |
| | naphthyridin-3- | |
| | yl)cyclopropanecarboxamide | |
| Example 31 | 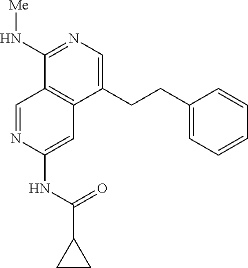 [SMILES: CNc1ncc(CCc2ccccc2)c2cc(NC(=O)C3CC3)ncc12] | LCMS (ESI) m/z 347.2, [M + H]⁺. ¹H NMR (400 MHz, DMSO-d₆) δ 10.99 (s, 1H), 9.32 (s, 1H), 8.46 (s, 1H), 7.77 (s, 1H), 7.76-7.70 (m, 1H), 7.32- 7.16 (m, 5H), 2.97-2.81 (m, 7H), 2.13-2.04 (m, 1H), 0.93-0.79 (m, 4H). |
| | N-(8-(methylamino)-5- | |
| | phenethyl-2,7-naphthyridin-3- | |
| | yl)cyclopropanecarboxamide | |
| Example 32 | 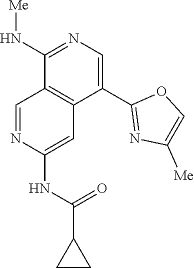 [SMILES: CNc1ncc(-c2nc(C)co2)c2cc(NC(=O)C3CC3)ncc12] | LCMS (ESI) m/z 324.1, [M + H]⁺. ¹H NMR (400 MHz, DMSO-d₆) δ 10.98 (s, 1H), 9.39 (s, 2H), 8.62 (s, 1H), 8.43- 8.37 (m, 1H), 7.87 (s, 1H), 3.04 (d, J = 4.4 Hz, 3H), 2.18 (s, 3H), 2.11- 2.03 (m, 1H), 0.90-0.80 (m, 4H). |
| | N-(8-(methylamino)-5-(4- | |
| | methyloxazol-2-yl)-2,7- | |
| | naphthyridin-3- | |
| | yl)cyclopropanecarboxamide | |
| Example 33 | 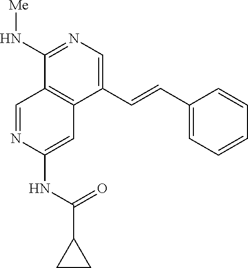 [SMILES: CNc1ncc(/C=C/c2ccccc2)c2cc(NC(=O)C3CC3)ncc12] | LCMS (ESI) m/z 345.2, [M + H]⁺. ¹H NMR (400 MHz, DMSO-d₆) δ 11.04 (s, 1H), 9.36 (s, 1H), 8.58 (s, 1H), 8.35 (s, 1H), 8.07-8.02 (m, 1H), 7.59- 7.55 (m, 2H), 7.43-7.35 (m, 3H), 7.30-7.24 (m, 1H), 7.08 (d, J = 16.0 Hz, 1H), 3.01 (d, J = 4.4 Hz, 3H), 2.11- 2.03 (m, 1H), 0.90-0.80 (m, 4H). |
| | (E)-N-(8-(methylamino)-5- | |
| | styryl-2,7-naphthyridin-3- | |
| | yl)cyclopropanecarboxamide | |
| Example 34 | 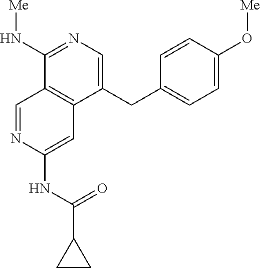 [SMILES: CNc1ncc(Cc2ccc(OC)cc2)c2cc(NC(=O)C3CC3)ncc12] | LCMS (ESI) m/z 363.2, [M + H]⁺. ¹H NMR (400 MHz, DMSO-d₆) δ 10.92 (s, 1H), 9.30 (s, 1H), 8.36 (s, 1H), 7.89 (s, 1H), 7.81-7.72 (m, 1H), 7.14- 7.10 (m, 2H), 6.82-6.77 (m, 2H), 3.91 (s, 2H), 3.68 (s, 3H), 2.95 (d, J = 4.4 Hz, 3H), 2.07-1.99 (m, 1H), 0.87-0.78 (m, 4H). |
| | N-(5-(4-methoxybenzyl)-8- | |
| | (methylamino)-2,7- | |
| | naphthyridin-3- | |
| | yl)cyclopropanecarboxamide | |
| Example 35 | 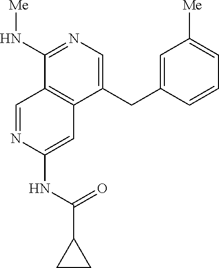 [SMILES: CNc1ncc(Cc2cccc(C)c2)c2cc(NC(=O)C3CC3)ncc12] | LCMS (ESI) m/z 333.2, [M + H]⁺. ¹H NMR (400 MHz, DMSO-d₆) δ 10.89 (s, 1H), 9.38 (s, 1H), 8.01-7.93 (m, 1H), 7.81 (s, 1H), 7.78 (s, 1H), 7.39- 7.25 (m, 3H), 7.19-7.13 (m, 1H), 3.02 (d, J = 4.4 Hz, 3H), 2.03 (s, 3H), 2.01-1.94 (m, 1H), 0.79-0.69 (m, 4H). |
| | N-(8-(methylamino)-5-(o- | |
| | tolyl)-2,7-naphthyridin-3- | |
| | yl)cyclopropanecarboxamide | |
| Example 36 | 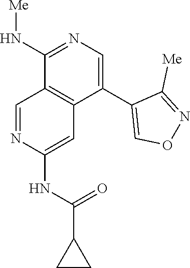 [SMILES: CNc1ncc(-c2conc2C)c2cc(NC(=O)C3CC3)ncc12] | LCMS (ESI) m/z 324.1, [M + H]⁺. ¹H NMR (400 MHz, DMSO-d₆) δ 11.02 (s, 1H), 9.38 (s, 1H), 8.93 (s, 1H), 8.12-8.06 (m, 1H), 8.02 (s, 1H), 7.95 (s, 1H), 3.01 (d, J = 4.4 Hz, 3H), 2.13 (s, 3H), 2.06-1.98 (m, 1H), 0.85- 0.78 (m, 4H). |
| | N-(8-(methylamino)-5-(3- | |
| | methylisoxazol-4-yl)-2,7- | |
| | naphthyridin-3- | |
| | yl)cyclopropanecarboxamide | |
| Example 37 | 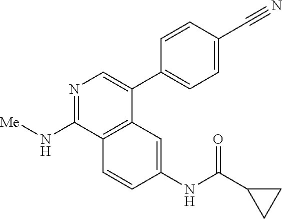 [SMILES: CNc1ncc(-c2ccc(C#N)cc2)c2cc(NC(=O)C3CC3)ccc12] | LCMS (ESI) m/z 344.1, [M + H]⁺. ¹H NMR (400 MHz, DMSO-d₆) δ 10.99 (s, 1H), 9.40 (s, 1H), 8.33 (s, 1H), 8.18-8.08 (m, 1H), 8.04-7.88 (m, 3H), 7.70-7.57 (m, 2H), 3.02 (d, J = 4.4 Hz, 3H), 2.09-1.95 (m, 1H), 0.90- 0.71 (m, 4H). |
| | N-(5-(4-cyanophenyl)-8- | |
| | (methylamino)-2,7- | |
| | naphthyridin-3- | |
| | yl)cyclopropanecarboxamide | |
| Example 38 | 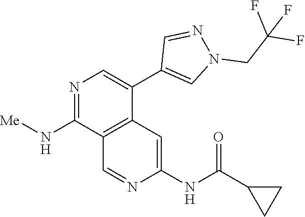 [SMILES: CNc1ncc(-c2cnn(CC(F)(F)F)c2)c2cc(NC(=O)C3CC3)ncc12] | LCMS (ESI) m/z 391.1, [M + H]⁺. ¹H NMR (400 MHz, DMSO-d₆) δ 10.97 (s, 1H), 9.36 (s, 1H), 8.47 (s, 1H), 8.03 (s, 1H), 8.00 (s, 1H), 7.96-7.91 (m, 1H), 7.78 (s, 1H), 5.25-5.16 (m, 2H), 2.99 (d, J = 4.4 Hz, 3H), 2.08- 2.00 (m, 1H), 0.87-0.78 (m, 4H). |
| | N-(8-(methylamino)-5-(1- | |
| | (2,2,2-trifluoroethyl)-1H- | |
| | pyrazol-4-yl)-2,7- | |
| | naphthyridin-3- | |
| | yl)cyclopropanecarboxamide | |

### Example 39: Synthesis of N-(5-(4-methyl-1-(tetrahydro-2H-pyran-2-yl)-1H-pyrazol-5-yl)-8-(methylamino)-2,7-naphthyridin-3-yl)cyclopropanecarboxamide

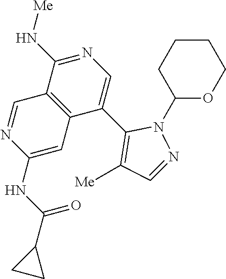 [SMILES: CNc1ncc(-c2c(C)cnn2C2CCCCO2)c2cc(NC(=O)C3CC3)ncc12]

To a stirring solution of N-(5-bromo-8-(methylamino)-2,7-naphthyridin-3-yl)cyclopropane-carboxamide (40 mg; 0.125 mmol; 1.00 eq.) in dioxane/water (5:1, 1.2 mL) under N₂ was added Pd(DtBPF)Cl₂ (8.1 mg; 0.012 mmol; 0.10 eq.), K₃PO₄(52.9 mg; 0.249 mmol; 2.00 eq.) and 4-methyl-1-(tetrahydro-2H-pyran-2-yl)-5-(4,4,5,5-tetramethyl-1,3,2-dioxaborolan-2-yl)-1H-pyrazole (72.8 mg; 0.249 mmol; 2.00 eq.) at room temperature. The reaction was stirred at 90° C. for 2 hours. The desired product could be detected by LCMS. The resulting mixture was concentrated under vacuum. The residue was purified by flash chromatography on silica gel column using MeOH/CH₂Cl₂ (1%-10%) as eluent to provide N-(5-(4-methyl-1-(tetrahydro-2H-pyran-2-yl)-1H-pyrazol-5-yl)-8-(methylamino)-2,7-naphthyridin-3-yl)cyclopropanecarboxamide (50 mg, crude). The crude product was purified by flash chromatography on pre-packed C18 column using 10%-70% of MeCN in water (10 mmol/L NH₄HCO₃) to provide N-(5-(4-methyl-1-(tetrahydro-2H-pyran-2-yl)-1H-pyrazol-5-yl)-8-(methylamino)-2,7-naphthyridin-3-yl)cyclopropanecarboxamide as a white solid (6.1 mg, 12%). LCMS (ESI) m/z 407.2, [M+H]⁺. ¹H NMR (400 MHz, DMSO-d₆) δ 10.96 (s, 1H), 9.39 (s, 1H), 8.24-8.11 (m, 1H), 7.96 (s, 1H), 7.78 (s, 1H), 7.50 (s, 1H), 4.84-4.75 (m, 1H), 3.93-3.81 (m, 1H), 3.41-3.29 (m, 1H), 3.03 (d, J=4.4 Hz, 3H), 2.40-2.26 (m, 1H), 2.05-1.70 (m, 6H), 1.54-1.30 (m, 3H), 0.88-0.70 (m, 4H).

### Example 40: Synthesis of N-(5-(4-methyl-1H-pyrazol-5-yl)-8-(methylamino)-2,7-naphthyridin-3-yl)cyclopropanecarboxamide

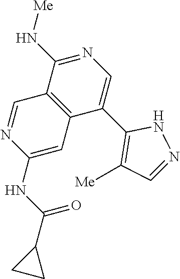 [SMILES: CNc1ncc(-c2[nH]ncc2C)c2cc(NC(=O)C3CC3)ncc12]

To a stirring mixture of N-(5-(4-methyl-1-(tetrahydro-2H-pyran-2-yl)-1H-pyrazol-5-yl)-8-(methyl amino)-2,7-naphthyridin-3-yl)cyclopropanecarboxamide (68 mg; 0.167 mmol; 1.00 eq.) in MeOH (1 mL) was added a solution of HCl in dioxane (4 M, 5 mL) at room temperature under nitrogen. The reaction was stirred at room temperature for 2 hours. The desired product was observed by LCMS. The resulting mixture was concentrated under vacuum. The residue was purified by flash chromatography on pre-packed C18 column using 10%-70% of MeCN in water (10 mmol/L NH₄CO₃) to provide N-(5-(4-methyl-1H-pyrazol-5-yl)-8-(methylamino)-2,7-naphthyridin-3-yl)cyclopropanecarboxamide as a white solid (10.5 mg, 19%). LCMS (ESI) m/z 323.2, [M+H]⁺. ¹H NMR (400 MHz, DMSO-d₆) δ 12.64 (s, 1H), 10.92 (s, 1H), 9.36 (s, 1H), 8.34-8.00 (m, 1H), 7.93 (s, 2H), 7.69-7.35 (m, 1H), 3.01 (d, J=4.4 Hz, 3H), 2.05-1.97 (m, 1H), 1.92 (s, 3H), 0.83-0.74 (m, 4H).

### Example 41 and Example 42: Synthesis of 2-(6-(cyclopropanecarboxamido)-1-(methylamino)-2,7-naphthyridin-4-yl)benzamide and N-(5-(2-cyanophenyl)-8-(methylamino)-2,7-naphthyridin-3-yl)cyclopropanecarboxamide

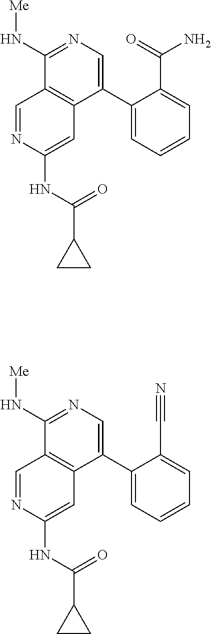 [SMILES: CNc1ncc(-c2ccccc2C(N)=O)c2cc(NC(=O)C3CC3)ncc12 ; CNc1ncc(-c2ccccc2C#N)c2cc(NC(=O)C3CC3)ncc12]

To a stirring solution of N-(5-bromo-8-(methylamino)-2,7-naphthyridin-3-yl)cyclopropane-carboxamide (100 mg; 0.311 mmol; 1.00 eq.) in 1,4-dioxane/water (10:1, 1.1 mL) were added (2-cyanophenyl)boronic acid (68.6 mg; 0.467 mmol; 1.5 eq.), Pd(dppf)Cl₂ (22.8 mg; 0.031 mmol; 0.10 eq.) and K₃PO₄ (132.2 mg; 0.622 mmol; 2.00 eq.). The reaction mixture was stirred for 1 h at 100° C. under nitrogen atmosphere. The resulting mixture was concentrated under reduced pressure. The residue was purified by flash chromatography on silica gel column using 25-60% of EtOAc in petroleum ether as eluent to afford 2-(6-(cyclopropanecarboxamido)-1-(methylamino)-2,7-naphthyridin-4-yl)benzamide (50 mg, crude) and N-(5-(2-cyanophenyl)-8-(methyl amino)-2,7-naphthyridin-3-yl)cyclopropanecarboxamide (15 mg, crude) separately. The two crude products were purified respectively by flash chromatography on pre-packed CIS column using 20-60% MeCN in water (10 mmol/L NH₄HCO₃) to provide 2-(6-(cyclopropanecarboxamido)-1-(methylamino)-2,7-naphthyridin-4-yl)benzamide as a white solid (28.8 mg, 26%) and N-(5-(2-cyanophenyl)-8-(methylamino)-2,7-naphthyridin-3-yl)cyclopropanecarboxamide as a white solid (4.3 mg, 4%). Analytical data for 2-(6-(cyclopropanecarboxamido)-1-(methylamino)-2,7-naphthyridin-4-yl)benzamide: LCMS (ESI) m/z 362.2, NMR (400 MHz, DMSO-d₆) δ 10.87 (s, 1H), 9.34 (s, 1H), 8.04 (s, 1H), 7.96-7.90 (m, 1H), 7.83 (s, 1H), 7.60-7.42 (m, 4H), 7.31 (dd, J=7.6, 1.2 Hz, 1H), 7.13 (s, 1H), 3.00 (d, J=4.4 Hz, 3H), 2.05-1.96 (m, 1H), 0.82-0.70 (m, 4H). Analytical data for N-(5-(2-cyanophenyl)-8-(methylamino)-2,7-naphthyridin-3-yl)cyclopropanecarboxamide: LCMS (ESI) m/z 344.1, [M+H]⁺. ¹H NMR (400 MHz, DMSO-d₆) δ 11.00 (s, 1H), 9.42 (s, 1H), 8.25-8.15 (m, 1H), 8.04-7.96 (m, 3H), 7.88-7.79 (m, 1H), 7.68-7.54 (2H), 3.04 (d, J=4.4 Hz, 3H), 2.05-1.95 (m, 1H), 0.81-0.70 (m, 4H).

### Example 43: Synthesis of N-(5-benzyl-8-(methylamino)-2,7-naphthyridin-3-yl)cyclopropanecarboxamide

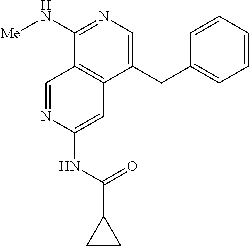 [SMILES: CNc1ncc(Cc2ccccc2)c2cc(NC(=O)C3CC3)ncc12]

To a stirring solution of N-(5-bromo-8-(methylamino)-2,7-naphthyridin-3-yl)cyclopropanecarboxamide (50 mg; 0.156 mmol; 1.00 eq.) in THF/water (10:1, 2.2 mL) were added potassium benzyltrifluoroborate (61.8 mg; 0.312 mmol; 2.00 eq.), Pd(OAc)₂ (3.5 mg; 0.016 mmol; 0.10 eq.), X-Phos (14.8 mg; 0.031 mmol; 0.20 eq.) and Cs₂CO₃ (101.4 mg; 0.312 mmol; 2.00 eq.). The reaction mixture was stirred for 12 h at 85 under nitrogen atmosphere. The reaction was monitored by LCMS. The resulting mixture was concentrated under reduced pressure. The residue was purified by flash chromatography on silica gel column using 0-10% of MeOH in CH₂Cl₂ as eluent to afford N-(5-benzyl-8-(methylamino)-2,7-naphthyridin-3-yl)cyclopropane-carboxamide (15 mg, crude). The crude product was purified by flash chromatography on pre-packed C18 column using 20-50% MeCN in water (10 mmol/L NH₄HCO₃) to provide N-(5-benzyl-8-(methylamino)-2,7-naphthyridin-3-yl)cyclopropanecarboxamide as a white solid (3.9 mg, 8%). LCMS (ESI) m/z 333.2, [M+H]⁺. ¹H NMR (400 MHz, DMSO-d₆) δ 10.93 (s, 1H), 9.31 (s, 1H), 8.37 (s, 1H), 7.93 (s, 1H), 7.83-7.75 (m, 1H), 7.30-7.21 (m, 4H), 7.19-7.10 (m, 1H), 3.99 (s, 2H), 2.96 (d, J=4.4 Hz, 3H), 2.10-2.00 (m, 1H), 0.90-0.75 (m, 4H).

### Example 44: Synthesis of N-(5-(hydroxymethyl)-8-(methylamino)-2,7-naphthyridin-3-yl)cyclopropanecarboxamide

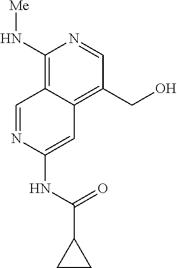 [SMILES: CNc1ncc(CO)c2cc(NC(=O)C3CC3)ncc12]

To a stirring mixture of N-(5-bromo-8-(methylamino)-2,7-naphthyridin-3-yl)cyclopropanecarboxamide (100 mg; 0.311 mmol; 1.00 eq.) in 1,4-dioxane (10 mL) were added Pd(PPh₃)₄ (36.1 mg; 0.031 mmol; 0.10 eq. and (tributylstannyl)methanol (300.9 mg; 0.937 mmol; 3.00 eq.). The reaction was stirred at 95° C. for 2.5 hours. The mixture was allowed to cool down to room temperature. The desired product was observed via LCMS. The solvent was removed under reduced pressure. The residue was purified by flash chromatography on pre-packed C18 column using 20%-70% MeCN in water (10 mmol/L NH₄HCO₃) as eluent to provide N-(5-(hydroxymethyl)-8-(methylamino)-2,7-naphthyridin-3-yl)cyclopropanecarboxamide as a white solid (60 mg, 70%). LCMS (ESI) m/z 273.1, [M+H]⁺. ¹H NMR (400 MHz, DMSO-d₆) δ 10.93 (s, 1H), 9.32 (s, 1H), 8.44 (s, 1H), 7.92 (s, 1H), 7.88-7.80 (m, 1H), 4.87 (t, J=5.0 Hz, 1H), 4.56 (d, J=5.0 Hz, 2H), 2.96 (d, 1=4.4 Hz, 3H), 2.12-2.01 (m, 1H), 0.91-0.78 (m, 4H).

### Example 45: Synthesis of N-(8-(methylamino)-5-(phenoxymethyl)-2,7-naphthyridin-3-yl)cyclopropanecarboxamide

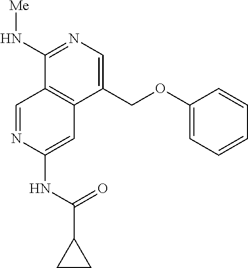 [SMILES: CNc1ncc(COc2ccccc2)c2cc(NC(=O)C3CC3)ncc12]

To a solution of N-(5-(hydroxymethyl)-8-(methylamino)-2,7-naphthyridin-3-yl)cyclopropanecarboxamide (30 mg; 0.110 mmol; 1.00 eq.) in THF (5 mL) were added phenol (104 mg; 1.10 mmol; 10.0 eq.), PPh₃ (55.7 mg; 0.212 mmol; 1.93 eq.) at room temperature. To this mixture reaction was added DIAD (43.3 mg; 0.214 mmol; 1.94 eq.) and stirred at room temperature for 1 h. The desired product was detected via LCMS. The solvent was removed under reduced pressure. The residue was purified by flash chromatography on silica gel column using MeOH/CH₂Cl₂ (2-10%) as eluent to provide N-(8-(methylamino)-5-(phenoxymethyl)-2,7-naphthyridin-3-yl)cyclopropanecarboxamide (10 mg, crude). The crude product was purified by reverse phase preparative HPLC (XBridge Prep OBD C18 Column, 30×150 mm, waters; gradient elution of 38%-48% MeCN in water over a 10 min period, where both water and MeCN contain 10 mmol/L NH₄HCO₃, flow rate: 60 mL/min, detector UV wavelength: 220 nm) to provide N-(8-(methylamino)-5-(phenoxymethyl)-2,7-naphthyridin-3-yl)cyclopropanecarboxamide as a white solid (1.4 mg, 3%). LCMS (ESI) m/z 349.2, [M+H]⁺. ¹H NMR (400 MHz, DMSO-d₆) δ 10.95 (s, 1H), 9.35 (s, 1H), 8.43 (s, 1H), 8.11 (s, 1H), 8.04-7.97 (m, 1H), 7.35-7.25 (m, 2H), 7.08-7.00 (m, 2H), 6.99-6.90 (m, 1H), 5.13 (s, 2H), 2.98 (d, J=4.4 Hz, 3H), 2.10-2.00 (m, 1H), 0.86-0.75 (m, 4H).

### Example 46: Synthesis of N-(8-(methylamino)-5-(4-(methylsulfonyl)phenyl)-2,7-naphthyridin-3-yl) cyclopropanecarboxamide

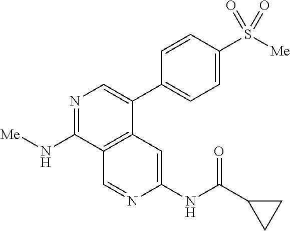 [SMILES: CNc1ncc(-c2ccc(S(C)(=O)=O)cc2)c2cc(NC(=O)C3CC3)ncc12]

To a stirring mixture of N-(5-bromo-8-(methylamino)-2,7-naphthyridin-3-yl)cyclopropanecarboxamide (30 mg; 0.093 mmol; 1.00 eq.) in 1,4-dioxane/water (5:1, 1.8 mL) were added XPhos (4.4 mg; 0.009 mmol; 0.10 eq.), XPhos Pd G3 (CAS: 1445085-55-1) (8.1 mg; 0.010 mmol; 0.10 eq.), K₃PO₄ (60 mg; 0.283 mmol; 2.00 eq.) and (4-(methylsulfonyl)phenyl)boronic acid (19 mg; 0.095 mmol; 1.00 eq.). The resulting solution was stirred under nitrogen atmosphere for 2 h at 90° C. The reaction was concentrated under vacuum. The residue was purified by flash chromatography on silica gel eluting methanol in dichloromethane (5-10%) as eluent to afford 50 mg of crude product. This crude was purified by flash chromatography on pre-packed C18 column using 20-50% of MeCN in water (10 mmol/L NH₄HCO₃) to provide N-(8-(methylamino)-5-(4-(methylsulfonyl)phenyl)-2,7-naphthyridin-3-yl)cyclopropanecarboxamide as a white solid (30.0 mg, 81%). LCMS (ESI) m/z 397.1, [M+H]⁺. ¹H NMR (400 MHz, DMSO-d₆) δ 10.99 (s, 1H), 9.41 (s, 1H), 8.37 (s, 1H), 8.15-8.12 (m, 1H), 8.05-8.02 (m, 2H), 8.01 (s, 1H), 7.73-7.69 (m, 2H), 3.30 (s, 3H), 3.03 (d, J=4.4 Hz, 3H), 2.06-1.98 (m, 1H), 0.82-0.76 (m, 4H).

### Example 47: Synthesis of N-(8-(methylamino)-5-(2-(methylthio)phenyl)-2,7-naphthyridin-3-yl)cyclopropanecarboxamide

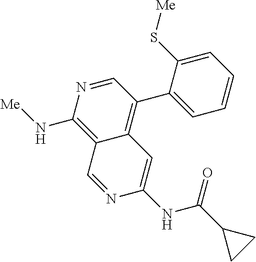 [SMILES: CNc1ncc(-c2ccccc2SC)c2cc(NC(=O)C3CC3)ncc12]

To a stilling mixture of N-(5-bromo-8-(methylamino)-2,7-naphthyridin-3-yl)cyclopropane-carboxamide (50 mg; 0.156 mmol; 1.00 eq.) in DME/water (5:1, 1.2 mL) were added (2-(methylthio)phenyl)boronic acid (78.5 mg; 0.467 mmol; 3.00 eq.), Pd(PPh₃)₄ (18.0 mg; 0.016 mmol; 0.10 eq.) and Na₂CO₃ (49.5 mg; 0.467 mmol; 3.00 eq.) at room temperature. The reaction was stirred under nitrogen at 100° C. for 3 h. The desired product was detected via LCMS. The resulting mixture was concentrated under vacuum. The residue was purified by flash chromatography on silica gel column using MeOH in CH₂Cl₂ (2-8%) as eluent to provide the 50 mg of the crude product. The crude product was purified by flash chromatography on pre-packed C18 column using 20-60% of MeCN in water (10 mmol/L NH₄HCO₃) to provide N-(8-(methylamino)-5-(2-(methylthio)phenyl)-2,7-naphthyridin-3-yl)cyclopropanecarboxamide as a white solid (3.9 mg, 6%). LCMS (ESI) m/z 365.1, [M+H]⁺. ¹H NMR (400 MHz, DMSO-d₆) δ 10.86 (s, 1H), 9.36 (s, 1H), 8.00-7.96 (m, 1H), 7.79 (s, 2H), 7.46-7.41 (m, 1H), 7.38-7.35 (m, 1H), 7.27-7.22 (m, 1H), 7.18-7.14 (m, 1H), 3.01 (d, J=4.4 Hz, 3H), 2.31 (s, 3H), 2.02-1.93 (m, 1H), 0.78-0.69 (m, 4H).

### Example 48: Synthesis of N-(8-(methylamino)-5-(2-(methylsulfonyl)phenyl)-2,7-naphthyridin-3-yl)cyclopropanecarboxamide

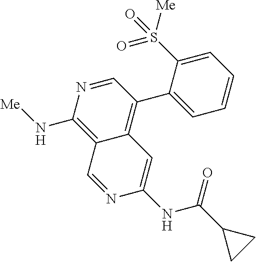 [SMILES: CNc1ncc(-c2ccccc2S(C)(=O)=O)c2cc(NC(=O)C3CC3)ncc12]

To a solution of N-(8-(methylamino)-5-(2-(methylthio)phenyl)-2,7-naphthyridin-3-yl)cyclopropanecarboxamide (14 mg; 0038 mmol; 1.00 eq.) in CH₂Cl₂ (1 mL) was added m-CPBA (13.3 mg; 0.077 mml; 2.00 eq.) at 0° C. The reaction was stirred under nitrogen at room temperature for 1 hour. The resulting mixture was concentrated under vacuum and purified by flash chromatography on silica gel column using 2-10% of MeOH in CH₂Cl₂ as eluent to provide N-(8-(methylamino)-5-(2-(methylsulfonyl)phenyl)-2,7-naphthyridin-3-yl)cyclopropanecarboxamide (8 mg, crude) and N-(8-(methylamino)-5-(2-(methylsulfinyl)phenyl)-2,7-naphthyridin-3-yl)cyclopropanecarboxamide (5 mg, crude) separately. The crude two products were purified respectively by flash chromatography on pre-packed C18 column using 20-60% of MeCN in water (10 mmol/L NH₄HCO₃) to provide N-(8-(methylamino)-5-(2-(methylsulfonyl)phenyl)-2,7-naphthyridin-3-yl)cyclopropanecarboxamide as a white solid (3.7 mg, 24%) and N-(8-(methylamino)-5-(2-(methylsulfinyl)phenyl)-2,7-naphthyridin-3-yl)cyclopropanecarboxamide as a white solid (0.8 mg, 5%). Analytical data for N-(8-(methylamino)-5-(2-(methylsulfonyl)phenyl)-2,7-naphthyridin-3-yl)cyclopropanecarboxamide: LCMS (ESI) m/z 397.1, [M+H]⁺. ¹H NMR (400 MHz, DMSO-d₆) δ 10.89 (s, 1H), 9.36 (s, 1H), 8.16-8.12 (m, 1H), 8.08-8.01 (m, 1H), 7.90 (s, 1H), 7.83-7.70 (m, 2H), 7.65 (s, 1H), 7.43-7.38 (m, 1H), 3.02 (d, J=4.4 Hz, 3H), 2.88 (s, 3H), 2.00-1.93 (m, 1H), 0.76-0.69 (m, 4H). Analytical data for N-(8-(methylamino)-5-(2-(methylsulfinyl)phenyl)-2,7-naphthyridin-3-yl)cyclopropanecarboxamide: LCMS (ESI) m/z 381.1, [M+H]⁺. ¹H NMR (400 MHz, DMSO-d₆) δ 10.96 (d, J=18.0 Hz, 1H), 9.39 (d, J=5.6 Hz, 1H), 8.17-8.08 (m, 1H), 7.99-7.61 (m, 5H), 7.35-7.30 (m, 1H), 3.02 (d, J=4.4, 3H), 2.56-2.25 (m, 3H), 2.02-1.94 (m, 1H), 0.79-0.74 (m, 4H).

### Example 49: Synthesis of N-(5-(2-methoxy-3-(1-methyl-1H-1,2,4-triazol-3-yl)phenyl)-8-(methylamino)-2,7-naphthyridin-3-yl)cyclopropanecarboxamide

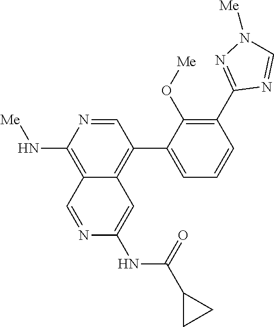 [SMILES: CNc1ncc(-c2cccc(-c3ncn(C)n3)c2OC)c2cc(NC(=O)C3CC3)ncc12]

### Step 1: 3-bromo-2-methoxybenzamide

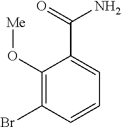 [SMILES: COc1c(Br)cccc1C(N)=O]

To a stirred solution of 3-bromo-2-methoxybenzoic acid (5.00 g; 21.6 mmol; 1.00 eq.) and DMF (0.321 g; 4.33 mmol; 0.20 eq.) in CH₂Cl₂ (50 mL) was added dropwise a solution of oxalyl dichloride (2 M) in CH₂Cl₂ (13.0 mL; 26.0 mmol; 1.20 eq.) at 0° C. The resulting mixture was stirred for 1 h at room temperature under nitrogen atmosphere. Then the resulting mixture was concentrated under reduced pressure. The crude product was added dropwise to a stirring solution of NH₃(g) (7 M) in MeOH (9.27 mL; 64.9 mmol; 3.00 eq.) at 0° C. The resulting mixture was stirred for 1 h at room temperature. The desired product was Observed via LCMS. The resulting mixture was concentrated under reduced pressure to provide 3-bronco-2-methoxybenzamide as a brown solid (4.98 g, 73%). LCMS (ESI) m/z: 230.0 [M+H]⁺.

### Step 2: 3-(3-bromo-2-methoxyphenyl)-1H-1,2,4-triazole

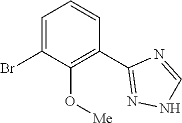 [SMILES: COc1c(Br)cccc1-c1nc[nH]n1]

3-bromo-2-methoxybenzamide (4.98 g; 21.7 mmol; 1.00 eq.) was dissolved in DMF-DMA (25.9 g; 217 mmol; 10.0 eq.). The resulting mixture was stirred under nitrogen for 0.5 h at 95° C. The resulting mixture was concentrated under reduced pressure to get the crude intermediate. The crude intermediate was dissolved in EtOH (10 mL) and then was added dropwise at 0° C. to a cold pre-treated solution (which contained a mixture of AcOH/EtOH (30 mL/120 mL) at 0° C. and hydrazine hydrate (80%) (10.6 mL; 217 mmol; 10.0 eq.)). The resulting reaction was stirred for 4 h at room temperature. The desired product was observed via LCMS. The reaction mixture was concentrated under reduced pressure. The resulting mixture was then added to the water (300 mL) and the precipitate solids were collected by filtration and washed with water (3×15 mL) and dried under reduced pressure to provide 3-(3-bromo-2-methoxyphenyl)-1H-1,2,4-triazole as a white solid (5.30 g, 96%). LCMS (ESI) m/z 254.0, [M+H]⁺.

### Step 3: 3-(3-bromo-2-methoxyphenyl)-1-methyl-1H-1,2,4-triazole

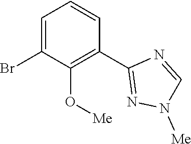 [SMILES: COc1c(Br)cccc1-c1ncn(C)n1]

A mixture of 3-(3-bromo-2-methoxyphenyl)-1H-1,2,4-triazole (5.30 g; 20.9 mmol; 1.00 eq.) and K₂CO₃ (80.65 g; 62.6 mmol; 3.00 eq.) in DMF (50 mL) was stirred for 30 min under nitrogen atmosphere. To the stirred mixture was added MeI (3.26 g; 22.9 mmol; 1.10 eq.) in DMF (10 mL) dropwise at 0° C. The final reaction mixture was stirred for 4 h at room temperature under nitrogen atmosphere. The desired product was observed via LCMS. The residue was purified by flash chromatography on silica gel column using 20%-80% of EtOAc in petroleum ether as eluent to provide 3-(3-bromo-2-triethoxyphenyl)-1-methyl-1H-1,2,4-triazole as a brown yellow oil (1.06 g, 19%). LCMS (ESI) m/z 268.0, [M+H]⁺. ¹H NMR (400 MHz, Methanol-d₆) δ 8.46 (s, 1H), 7.80 (dd, J=10.4, 2.4 Hz, 1H), 7.66 (dd, or J=10.4, 2.4 Hz, 1H), 7.12 (t, J=10.4 Hz, 1H), 4.00 (s, 3H), 3.76 (s, 3H).

### Step 4: 3-(2-methoxy-3-(4,4,5,5-tetramethyl-1,3,2-dioxaborolan-2-yl)phenyl)-1-methyl-1H-1,2,4-triazole

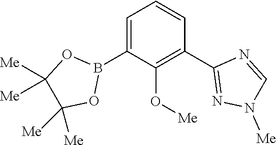 [SMILES: COc1c(B2OC(C)(C)C(C)(C)O2)cccc1-c1ncn(C)n1]

A mixture of 3-(3-bromo-2-methoxyphenyl)-1-methyl-1H-1,2,4-triazole (300 mg; 1.12 mmol; 1.00 eq.), 4,4,4′,4′,5,5,5′,5′-octamethyl-2,2′-bi(1,3,2-dioxaborolane) (852 mg; 3.36 mmol; 3.00 eq.), Pd(dppf)Cl₂·CH₂Cl₂ (91.7 mg; 0.113 mmol; 0.10 eq.) and KOAc (221 mg; 2.25 mmol; 2.01 eq.) in 1,4-dioxane (5 mL) was stirred for 2 h at 100° C. under nitrogen atmosphere. The desired product was observed via LCMS. The resulting mixture was concentrated under reduced pressure.

A mixture solvent of petroleum ether/EtOAc (10:1, 33 mL) was added and the solids were formed. The mixture was stirred for 2 h at room temperature. The precipitate solids were filtered with filter paper, the solids was dried under reduced pressure to provide 3-(2-methoxy-3-(4,4,5,5-tetramethyl-1,3,2-dioxaborolan-2-yl)phenyl)-1-methyl-1H-1,2,4-triazole as a brown solid (528 mg, crude). LCMS (ESI) m/z 316.2, [M+H]⁺.

### Step 5: N-(5-(2-methoxy-3-(1-methyl-1H-1,2,4-triazol-3-yl)phenyl)-8-(methylamino)-2,7-naphthyridin-3-yl)cyclopropanecarboxamide

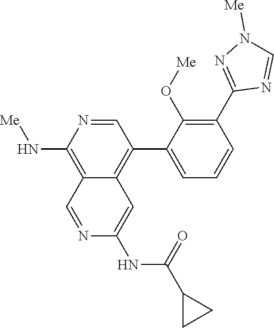 [SMILES: CNc1ncc(-c2cccc(-c3ncn(C)n3)c2OC)c2cc(NC(=O)C3CC3)ncc12]

A mixture of Na₂CO₃ (49.7 mg; 0.469 mmol; 3.00 eq.), 3-(2-methoxy-3-(4,4,5,5-tetramethyl-1,3,2-dioxaborolan-2-yl)phenyl)-1-methyl azole (crude, 57% purity) (256.3 mg; 0.468 mmol; 3.00 eq.), N-(5-bromo-8-(methylamino)-2,7-naphthyridin-3-yl)cyclopropanecarboxamide (50 mg; 0.156 mmol; 1.00 eq.) and Pd(PPh₃)₄ (18.1 mg; 0.016 mmol; 0.10 eq.) in DME/water (5:1, 1.2 mL) was stirred at 100° C. for 2 h under nitrogen atmosphere. The mixture was allowed to cool down to room temperature. The resulting mixture was concentrated under reduced pressure. The residue was purified by flash chromatography on silica gel column using MeOH in CH₂Cl₂ (2-5%) as eluent to provide N-(5-(2-methoxy-3-(1-methyl-1H-1,2,4-triazol-3-yl)phenyl)-8-(methylamino)-2,7-naphthyridin-3-yl)cyclopropanecarboxamide as a yellow solid (50 mg, crude). The crude product was purified by flash chromatography on pre-packed C18 column using 20%-50% MeCN in water (0.05% formic acid) as eluent to provide N-(5-(2-methoxy-3-(1-methyl-1H-1,2,4-triazol-3-yl)phenyl)-8-(methylamino)-2,7-naphthyridin-3-yl)cyclopropanecarboxamide formate as a white solid (35.5 mg, 53%). LCMS (ESI) m/z 430.2, [M+H]⁺. ¹H NMR (400 MHz, DMSO-d₆) δ 10.87 (s, 1H), 9.38 (s, 1H), 8.55 (s, 1H), 8.02-7.91 (m, 3H), 7.89-7.84 (m, 7.30 (s, 1H), 7.29 (s, 1H), 3.94 (s, 3H), 3.35 (s, 3H) 3.03 (d, J=4.4 Hz, 3H), 2.04-1.93 (m, 1H)), 0.78-0.71 (m, 4H).

### Example 50: Synthesis of N-(8-(methylamino)-5-(pyrimidin-4-yl)-2,7-naphthyridin-3-yl)cyclopropanecarboxamide

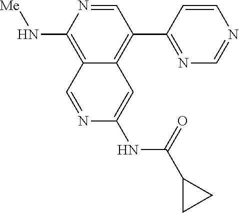 [SMILES: CNc1ncc(-c2ccncn2)c2cc(NC(=O)C3CC3)ncc12]

A mixture of CuI (3.6 mg; 0.019 mmol; 0.15 eq.), N-(5-bromo-8-(methylamino)-2,7-naphthyridin-3-yl)cyclopropanecarboxamide (40 mg; 0.125 mmol; 1.00 eq.) and Pd(PPh₃)₂Cl₂ (18.4 mg; 0.026 mmol; 0.21 eq.) in 1,4-dioxane (4 mL) was stirred at rt. To this mixture was added 4-(tributylstannyl)pyrimidine (138.8 mg; 0.376 mmol; 3.00 eq.). The reaction was stirred under nitrogen at 120° C. for 5 h. Then the additional of 4-(tributylstannyl)pyrimidine (138.8 mg; 0.376 mmol; 3.00 eq.) was added. The reaction was stirred at 120° C. overnight. The mixture was allowed to cool down to room temperature. The resulting mixture was concentrated under reduced pressure. The residue was purified by flash chromatography on silica gel column using MeOH in CH₂Cl₂ (1-5%) as eluent to provide the crude product (30 mg). The crude product was purified by flash chromatography on pre-packed C18 column using 20-50% MeCN in water (10 mmol/C, NH₄HCO₃) as eluent to provide N-(8-(methylamino)-5-(pyrimidin-4-yl)-2,7-naphthyridin-3-yl)cyclopropanecarboxamide as a white solid (15.1 mg, 37%), LCMS (ESI) m/z 321.1 [M+H]⁺. ¹H NMR. (400 MHz, DMSO-d₆) δ 10.98 (s, 1H), 9.41 (s, 1H), 9.23 (s, 1H), 8.99 (s, 1H), 8.83 (d, J=5.2 Hz, 1H), 8.42-8.32 (m, 2H), 7.78 (d, J=4.8 Hz, 1H), 3.06 (d, 4.0 Hz, 3H), 2.09-2.00 (m, 1H), 0.87-0.78 (m, 4H).

### Example 51: Synthesis of N-(8-(methylamino)-5-(1-(tetrahydro-2H-pyran-2-3H)-1H-pyrazol-5-yl)-2,7-naphthyridin-3-yl)cyclopropanecarboxamide

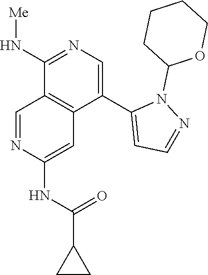 [SMILES: CNc1ncc(-c2ccnn2C2CCCCO2)c2cc(NC(=O)C3CC3)ncc12]

A mixture of N-(5-bromo-8-(methylamino)-2,7-naphthyridin-3-yl)cyclopropanecarboxamide (30 mg; 0.093 mmol; 1.00 eq.), 1-(tetrahydro-2H-pyran-2-yl)-5-(4,4,5,5-tetramethyl-1,3,2-dioxaborolan-2-yl)-1H-pyrazole (77.9 mg; 0.28 mmol; 3.00 eq.), Pd(PPh₃)₄. (10.8 mg; 0.009 mmol; 0.10 eq.) and Na₂CO₃(53.5 mg, 0.505 mmol, 5.40 eq.) in DME/water (5:1, 3.0 mL) was stirred for 2 h at 100° C. under nitrogen atmosphere. The desired product was observed via LCMS. The resulting mixture was concentrated under reduced pressure. The residue was purified by flash chromatography on silica gel column using 3-20% of MeOH in CH₂Cl₂ as eluent to provide N-(8-(methylamino)-5-(1-(tetrahydro-2H-pyran-2-yl)-1H-pyrazol-5-yl)-2,7-naphthyridin-3-yl)cyclopropanecarboxamide as a brown solid (26.9 mg, crude). The crude product was purified by flash chromatography on pre-packed C18 column using 20-50% of MeCN in water (10 mmol/L, NH₄HCO₃) as eluent to provide N-(8-(methylamino)-5-(1-(tetrahydro-2H-pyran-2-yl)-1H-pyrazol-5-yl)-2,7-naphthyridin-3-yl)cyclopropanecarboxamide as a white solid (10.6 mg, 28%). LCMS (ESI) m/z 393.2, [M+H]⁺. ¹H NMR (400 MHz, DMSO-d₆) δ 10.95 (s, 1H), 9.38 (s, 1H), 8.19-8.14 (m, 1H), 8.08 (s, 1H), 7.99 (s, 1H), 7.65 (d, J=1.6 Hz, 1H), 6.35 (d, J=1.6 Hz, 1H), 5.04-4.99 (m, 1H), 3.88-3.79 (m, 1H), 3.29-3.25 (m, 1H), 3.03 (d, J=4.4 Hz, 3H), 2.40-2.29 (m, 1H), 2.05-196 (m, 1H), 1.93-1.79 (m, 2H), 1.54-1.36 (m, 3H), 0.83-0.78 (m, 4H).

### Example 52: Synthesis of N-(8-(methylamino)-5-(1H-pyrazol-5-yl)-2,7-naphthyridin-3-yl)cyclopropanecarboxamide

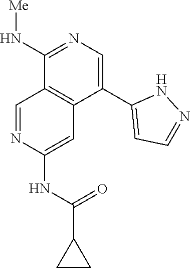 [SMILES: CNc1ncc(-c2ccn[nH]2)c2cc(NC(=O)C3CC3)ncc12]

To a solution of N-(8-(methylamino)-5-(1-(tetrahydro-2H-pyran-2-yl)-1H-pyrazol-5-yl)-2,7-naphthyridin-3-yl)cyclopropanecarboxamide (53.3 mg 0.136 mmol; 1.00 eq.) in MeOH (1 mL) was added a solution of HCl in dioxane (4 M, 4 mL). The reaction was stirred for 2 h at room temperature. The desired product was observed via LCMS. The resulting mixture was concentrated under reduced pressure. The residue was purified by flash chromatography on pre-packed C18 column using 20-40% of MeCN in water (10 mmol/L NH₄HCO₃) as eluent to provide N-(8-(methyl amino)-5-(1H-pyrazol-5-yl)-2,7-naphthyridin-3-yl)cyclopropane-carboxamide as a white solid (26.2 mg, 62%). LCMS (ESI) m/z 309.1, [M+H]⁺. ¹H NMR (400 MHz, DMSO-d₆) δ 12.92 (s, 1H), 10.88 (s, 1H), 9.36 (s, 1H), 9.11-8.43 (m, 1H), 8.27-7.53 (m, 3H), 6.58-6.41 (m, 1H), 3.01 (d, J=4.4 Hz, 3H), 2.08-2.00 (m, 1H), 0.86-0.78 (m, 4H).

### Example 53: Synthesis of N-(5-(3-(1-methyl-1H-1,2,4-triazol-3-yl)phenyl)-8-(methylamino)-2,7-naphthyridin-3-yl)cyclopropanecarboxamide

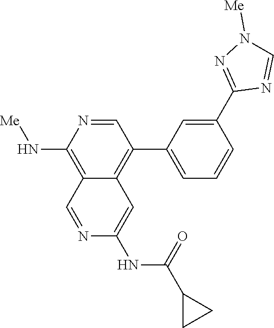 [SMILES: CNc1ncc(-c2cccc(-c3ncn(C)n3)c2)c2cc(NC(=O)C3CC3)ncc12]

### Step 1: 3-(3-bromophenyl)-1-methyl-1H-1,2,4-triazole and 5-(3-bromophenyl)-1-methyl-1H-1,2,4-triazole

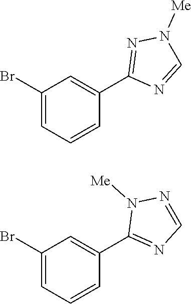 [SMILES: Cn1cnc(-c2cccc(Br)c2)n1 ; Cn1ncnc1-c1cccc(Br)c1]

To a stirring mixture of 3-(3-bromophenyl)-1H-1,2,4-triazole (1 g; 4.46 mmol: 1.00 eq.) in DMF (10 mL) at 0° C. was added K₂CO₃ (1.88 g; 13.63 mmol; 3.00 eq.) followed by a solution of CH₃I (697 mg; 4.91 mmol; 1.10 eq.) in DMF (1 mL) dropwise. The reaction mixture was added to a sat. NH₄Cl solution (30 mL). The resulting mixture was extracted with EtOAc (15 mL×2). The combined organic layers were washed with a sat. NaCl solution (5 mL×2), dried over anhydrous Na₂SO₄. After filtration, the filtrate was concentrated under reduced pressure. The residue was purified by flash chromatography on silica gel column using 30-70% of EtOAc in petroleum ether as eluent to provide 3-(3-bromophenyl)-1-methyl-1H-1,2,4-triazole as a yellow oil (732 mg, 68%) and 5-(3-bromophenyl)-1-methyl-1H-1,2,4-triazole as a yellow oil (70 mg, 6%). LCMS (ESI) m/z 238.0, [M+H]⁺. HNMR for 3-(3-bromophenyl)-1-methyl-1H-1,2,4-triazole: ¹H NMR (400 MHz, DMSO-d₆) δ 8.56 (s, 1H), 8.13-8.09 (m, 1H), 8.00-7.95 (m, 1H), 7.64-7.59 (m, 1H), 7.57-7.40 (m, 1H), 3.94 (s, 3H). HNMR for 5-(3-bromophenyl)-1-methyl-1H-1,2,4-triazole: ¹H NMR (400 MHz, DMSO-d₆) δ 8.03 (s, 1H), 7.98-7.95 (m, 1H), 7.82-7.74 (m, 2H), 7.56-7.51 1H), 3.99 (s, 3H).

### Step 2: 1-methyl-3-(3-(4,4,5,5-tetramethyl-1,3,2-dioxaborolan-2-yl)phenyl)-1H-1,2,4-triazole

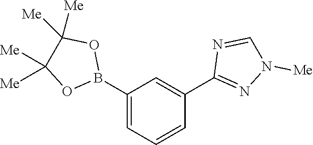 [SMILES: Cn1cnc(-c2cccc(B3OC(C)(C)C(C)(C)O3)c2)n1]

A mixture of 3-(3-bromophenyl)-1-methyl-1H-1,2,4-triazole (200 mg; 0.84 mmol; 1.00 eq.) and 4,4,4′,4′,5,5,5′,5′-octamethyl-2,2′-bi(1,3,2-dioxaborolane) (640 mg; 2.52 mmol; 3.00 eq.), Pd(dppf)Cl₂ (69 mg; 0.09 mmol; 0.10 eq.), KOAc (165 mg; 1.68 mmol; 2.00 eq.) in 1,4-dioxane (4 mL) was stirred for 2 h at 100° C. under nitrogen atmosphere. The solvent was concentrated under reduced pressure. The residue was dissolved in a mixture solvent of petroleum ether/EtOAc(10:1, 30 mL). The resulting mixture was filtered, the filter cake was washed with a mixture solvent of petroleum ether/EtOAc (10:1, 20 mL). The filtrate was concentrated under reduced pressure to provide 1-methyl-3-(3-(4,4,55-tetramethyl-1,3,2-dioxaborolan-2-yl)phenyl)-1H-1,2,4-triazole as a brown solid (350 mg, crude). LCMS (ESI) m/z 286.2, [M+H]⁺.

### Step 3: N-(5-(3-(1-methyl-1H-1,2,4-triazol-3-yl)phenyl)-8-(methylamino)-2,7-naphthyridin-3-yl)cyclopropanecarboxamide

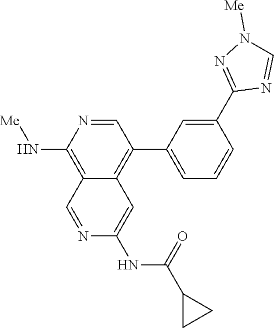 [SMILES: CNc1ncc(-c2cccc(-c3ncn(C)n3)c2)c2cc(NC(=O)C3CC3)ncc12]

A mixture of N-(5-(promo-8-(methylamino)-2,7-naphthyridin-3-yl)cyclopropanecarboxamide (40 mg; 0.12 mmol; 0.10 eq.), 1-methyl-3-(3-(4,4,5,5-tetramethyl-1,3,2-dioxaborolan-2-yl)phenyl)-1H-1,2,4-triazole (106.8 mg; 0.374 mmol; 3.00 eq.), Pd(PPh₃)₄ (14 mg; 0.01 mmol; 0.10 eq.), Na₂CO₃ (40 mg; 0.37 mmol; 3.00 eq.) in DME/water (5:1, 2.4 mL) was stirred for 3 h at 100° C., under nitrogen atmosphere. Upon completion, the resulting mixture was concentrated under reduced pressure. The residue was purified by flash chromatography on silica gel column using MeOH in CH₂Cl₂ (2-6%) as eluent to provide N-(5-(3-(1-methyl-1H-1,2,4-triazol-3-yl)phenyl)-8-(methylamino)-2,7-naphthyridin-3-yl)cyclopropanecarboxamide (20 mg, crude). The residue was purified by flash chromatography on pre-packed C18 column using 20-60% of MeCN in water (10 mmol/L NH₄HCO₃) to provide N-(5-(3-(1-methyl-1H-1,2,4-triazol-3-yl)phenyl)-8-(methylamino)-2,7-naphthyridin-3-yl)cyclopropanecarboxamide as a white solid (8.3 mg, 2%). LCMS (ESI) m/z 400.2, [M+H]⁺. ¹H NMR (400 MHz, DMSO-d₆) δ 11.00 (s, 1H), 9.44 (s, 1H), 8.54 (s, 1H), 8.35-8.31 (m, 1H), 8.31 (s, 1H), 8.05-8.0.1 (m, 1H), 7.98 (s, 1H), 7.92 (s, 1H), 7.61-7.55 (m, 1H), 7.47-7.42 (m, 1H), 3.93 (s, 3H), 3.05 (d, 4.4 Hz, 3H), 2.04-1.95 (m, 1H), 0.79-0.72 (m, 4H).

### Example 54: Synthesis of N-(5-(3-(1-methyl-1H-1,2,4-triazol-5-yl)phenyl)-8-(methylamino)-2,7-naphthyridin-3-yl)cyclopropanecarboxamide

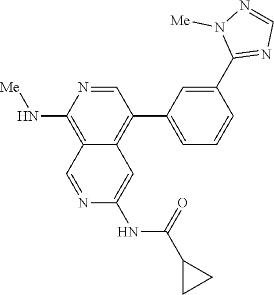 [SMILES: CNc1ncc(-c2cccc(-c3ncnn3C)c2)c2cc(NC(=O)C3CC3)ncc12]

### Step 1: 1-methyl-5-(3-(4,4,5,5-tetramethyl-1,3,2-dioxaborolan-2-yl)phenyl)-1-1,2,4-triazole

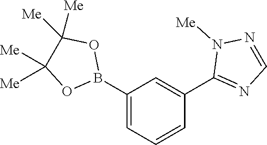 [SMILES: Cn1ncnc1-c1cccc(B2OC(C)(C)C(C)(C)O2)c1]

A mixture of 5-(3-bromophenyl)-1-methyl-1H-1,2,4-triazole (70 mg; 0.295 mmol; 1.00 eq.) and 4,4,4′,4′,5,5,5′,5′-octamethyl-2,2′-bi(1,3,2-dioxaborolane) (225 mg; 0.885 mmol; 3.00 eq.), Pd(dppf)Cl₂ (21.5 mg; 0.029 mmol; 0.10 eq.), KOAc (57.8 mg; 0.589 mmol; 2.00 eq.) in 1,4-dioxane (4 mL) was stirred for 2 h at 100° C. under nitrogen atmosphere. Upon completion, the reaction was concentrated under reduced pressure. The residue was dissolved in a mixture solvent of petroleum ether/EtOAc(10:1, 20 mL). The resulting mixture was filtered, the filter cake was washed with a mixture solvent of petroleum ether/EtOAc (10:1, 10 mL). The filtrate was concentrated under reduced pressure to provide 1-methyl-5-(3-(4,4,5,5-tetramethyl-1,3,2-dioxaborolan-2-yl)phenyl)-1H-1,2,4-triazole (100 mg, crude). The crude product was purified by flash chromatography on silica gel column using 30%-60% of EtOAc in petroleum ether as eluent to afford 1-methyl-5-(3-(4,4,5,5-tetramethyl-1,3,2-dioxaborolan-2-yl)phenyl)-1H-1,2,4-triazole as a brown solid (40 mg, 47%). LCMS (ESI) m/z 286.2, [M+H]⁺.

### Step 2: N-(5-(3-(1-methyl-1H-1,2,4-triazol-5-yl)phenyl)-8-(methylamino)-2,7-naphthyridin-3-yl)cyclopropanecarboxamide

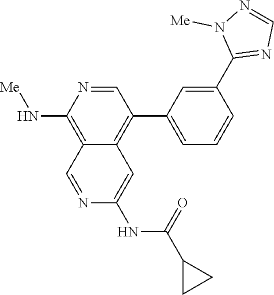 [SMILES: CNc1ncc(-c2cccc(-c3ncnn3C)c2)c2cc(NC(=O)C3CC3)ncc12]

A mixture of N-(5-bromo-8-(methylamino)-2,7-naphthyridin-3-yl)cyclopropanecarboxamide (40 mg; 0.12 mmol; 0.10 eq.), 1-methyl-5-(3-(4,4,5,5-tetramethyl-1,3,2-dioxaborolan-2-yl)phenyl)-1H-1,2,4-triazole (35.6 mg; 1.24 mmol; 1.00 eq.), Pd(PPh₃)₄ (14 mg; 0.01 mmol; 0.10 eq.), Na₂CO₃(39.7 mg; 0.37 mmol; 3.00 eq.) in DME/water (5:1, 2.4 mL) was stirred for 3 h at 100° C. under nitrogen atmosphere. After cooling down, the resulting mixture was concentrated under reduced pressure. The residue was purified by flash chromatography on silica gel column using MeOH in CH₂Cl₂ (2-6%) as eluent to provide N-(5-(3-(1-methyl-1H-1,2,4-triazol-5-yl)phenyl)-8-(methylamino)-2,7-naphthyridin-3-yl)cyclopropanecarboxamide (25 mg, crude). The crude product was purified by flash chromatography on pre-packed C18 column using 20%-60% of MeCN in water (10 mmol/L NH₄HCO₃) to provide N-(5-(3-(1-methyl-1H-1,2,4-triazol-5-yl)phenyl)-8-(methylamino)-2,7-naphthyridin-3-yl)cyclopropanecarboxamide as a white solid (16.7 mg, 29%). LCMS (ESI) m/z 400.2, [M+H]⁺. ¹H NMR (400 MHz, DMSO-d₆) δ 10.99 (s, 1H), 9.40 (s, 1H), 8.39 (s, 1H), 8.07-8.02 (m, 1H) 8.01 (s, 1H), 8.01 (s, 1H), 7.82-7.76 (m, 2H), 7.70-7.65 (m, 1H), 7.61-7.57 (m, 1H), 4.02 (s, 3H), 3.02 (d, J=4.4 Hz, 3H), 2.05-1.98 (m, 1H), 0.80-0.74 (m, 4H).

### Example 55: Synthesis of N-(8-(methylamino)-5-(pyrrolidin-1-yl)-2,7-naphthyridin-3-yl)cyclopropanecarboxamide

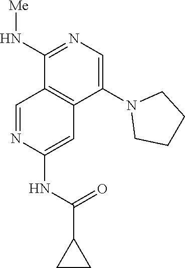 [SMILES: CNc1ncc(N2CCCC2)c2cc(NC(=O)C3CC3)ncc12]

A mixture of N-(5-bromo-8-(methylamino)-2,7-naphthyridin-3-yl)cyclopropanecarboxamide (40 mg; 0.125 mmol; 1.00 eq.), Pd-PEPPSI-IHeptCl (CAS: 1814936-54-3) (12.2 mg; 0.013 mmol; 0.10 eq.), Cs₂CO₃ (122.3 mg; 0.375 mmol; 3.00 eq.) and pyrrolidine (17.8 mg; 0.250 mmol; 2.00 eq.) in 1,4-dioxane (4 mL) was stirred at 100° C. for 16 h under nitrogen. The mixture was allowed to cool down to room temperature. The desired product was observed via LCMS. The residue was purified by flash chromatography on silica gel column using MeOH in CH₂Cl₂ (2-10%) as eluent to provide N-(8-(methylamino)-5-(pyrrolidin-1-yl)-2,7-naphthyridin-3-yl)cyclopropane-carboxamide (40 mg, crude). The crude product was purified by flash chromatography on pre-packed C18 column using 20%-50% MeCN in water (10 mmol/L NH₄HCO₃) as eluent to provide N-(8-(methylamino)-5-(pyrrolidin-1-yl)-2,7-naphthyridin-3-yl)cyclopropanecarboxamide as a yellow solid (23.5 mg, 60%). LCMS (ESI) m/z 312.2, [M+H]⁺. ¹H NMR (400 MHz, DMSO-d₆) δ 10.95 (s, 1H), 9.28 (s, 1H), 8.48 (s, 1H), 7.71 (s, 1H), 7.52-7.41 (m, 1H), 3.12-3.04 (m, 4H), 2.92 (d, J=4.4 Hz, 3H), 2.11-2.01 (m, 1H), 1.98-1.85 (m, 4H), 0.91-0.78 (m, 4H).

### Example 56: Synthesis of N-(8-(methylamino)-5-(piperidin-1-yl)-2,7-naphthyridin-3-yl)cyclopropanecarboxamide

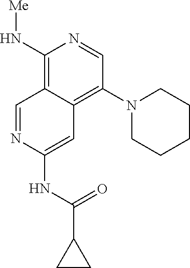 [SMILES: CNc1ncc(N2CCCCC2)c2cc(NC(=O)C3CC3)ncc12]

To a solution of N-(5-bromo-8-(methylamino)-2,7-naphthyridin-3-yl)cyclopropanecarboxamide (40 mg; 0.125 mmol; 1.00 eq.) in dioxane (2.00 mL) were added Pd-PEPPSI-IHeptCl (12.1 mg; 0.012 mmol; 0.10 eq.), Cs₂CO₃ (121.7 mg; 0.374 mmol; 3.00 eq.) and piperidine (21.2 mg; 0.249 mmol; 2.00 eq.) at room temperature. The reaction was stirred under nitrogen at 100° C. for 16 h. Upon completion, the resulting mixture was concentrated under vacuum. The residue was purified by flash chromatography on silica gel column using MeOH in CH₂Cl₂ (1-9%) as eluent to provide N-(8-(methylamino)-5-(piperidin-1-yl)-2,7-naphthyridin-3-yl)cyclopropane-carboxamide (30 mg, crude). The residue was purified by flash chromatography on pre-packed C18 column using 10%-70% of MeCN in water (10 mmol/L NH₄HCO₃) to provide N-(8-(methylamino)-5-(piperidin-1-yl)-2,7-naphthyridin-3-yl)cyclopropanecarboxamide as a white solid (21.9 mg, 54%). LCMS (ESI) m/z 326.2, [M+H]⁺. ¹H NMR (400 MHz, DMSO-d₆) δ 10.91 (s, 1H), 9.28 (s, 1H), 8.46 (s, 1H), 7.68 (s, 1H), 7.61-7.53 (m, 1H), 2.92 (d, J=4.4 Hz, 3H), 2.90-2.77 (m, 4H), 2.10-2.09 (m, 1H), 1.73-1.64 (m, 4H), 1.60-1.50 (m, 2H), 0.90-0.79 (m, 4H).

### Example 57: Synthesis of N-(8-(methylamino)-5-(1-pyrazol-1-yl)-2,7-naphthyridin-3-yl)cyclopropanecarboxamide

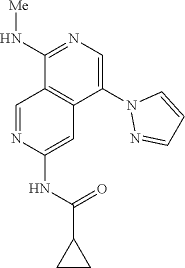 [SMILES: CNc1ncc(-n2cccn2)c2cc(NC(=O)C3CC3)ncc12]

To a mixture of N-(5-bromo-8-(methylamino)-2,7-naphthyridin-3-yl)cyclopropanecarboxamide (50 mg; 0.156 mmol; 1.00 eq.), CuI (29.6 mg; 0.158 mmol; 1.00 eq.), K₂CO₃ (43.1 mg; 0.312 mmol, 2.00 eq.) and (1R,2R)—N¹,N²-dimethylcyclohexane-1,2-diamine (40 mg; 0.281 mmol; 1.80 eq.) in DUE (5 mL, deoxygenated prior to use) was added 1H-pyrazole (13.0 mg; 0.191 mmol; 1.23 eq.). The reaction was stirred under nitrogen at 120° C. for 16 h. The mixture was allowed to cool down to room temperature. The desired product was observed via LCMS. The resulting mixture was diluted with EtOAc (70 mL), and then was washed with a saturated NaCl solution (5×10 mL). The organic phase was dried with Na₂SO₄, filtered and the solvent was removed under reduced pressure. The residue was purified by flash chromatography on silica gel column using MeOH/CH₂Cl₂ (2-10%) as eluent to provide N-(8-(methylamino)-5-(1H-pyrazol-1-yl)-2,7-naphthyridin-3-yl)cyclopropanecarboxamide (20 mg, crude). The crude product was purified by flash chromatography on pre-packed C18 column using 20%-50% MeCN in water (0.05% formic acid) as eluent to provide N-(8-(methyl amino)-5-(1H-pyrazol-1-yl)-2,7-naphthyridin-3-yl)cyclopropanecarboxamide as a white solid (10.6 mg, 22%). LCMS (ESI) m/z 309.1, [M+H]⁺. ¹H NMR (400 MHz, DMSO-d₆) δ 11.00 (s, 1H), 9.40 (s, 1H), 8.25-8.18 (m, 1H), 8.05 (s, 1H), 8.04 (s, 1H), 7.97 (d, J=2.4 Hz, 1H), 7.76 (d, J=1.8 Hz, 1H), 6.52 (t, J=2.1 Hz, 1H), 3.03 (d, J=4.3 Hz, 3H), 2.07-1.96 (m, 1H), 0.82-0.75 (m, 4H).

### Example 58: Synthesis of N-(5-(1,1-dioxidoisothiazolidin-2-yl)-8-(methylamino)-2,7-naphthyridin-3-yl)cyclopropanecarboxamide

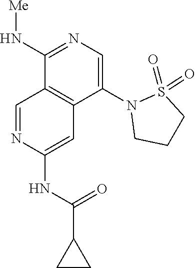 [SMILES: CNc1ncc(N2CCCS2(=O)=O)c2cc(NC(=O)C3CC3)ncc12]

A mixture of N-(5-bromo-8-(methylamino)-2,7-naphthyridin-3-yl)cyclopropanecarboxamide (40 mg; 0.125 mmol; 1.00 eq.), isothiazolidine 1,1-dioxide (15.1 mg; 0.124 mmol; 1.00 eq.), Pd-PEPPSI-IPentCl (10.9 mg; 0.012 mmol; 0.10 eq.) and Cs₂CO₃ (81.5 mg; 0.25 mmol; 2.00 eq.) in 1,4-dioxane (2 mL) was stirred for 12 h at 120° C. under nitrogen atmosphere. The reaction was monitored by LCMS. The residue was purified by flash chromatography on silica gel column using MeOH/CH₂Cl₂ (2-10%) as eluent to afford N-(5-(1,1-dioxidoisothiazolidin-2-yl)-8-(methylamino)-2,7-naphthyridin-3-yl)cyclopropanecarboxamide (12 mg, crude). The crude product was purified by flash chromatography on pre-packed C18 column using 20-50% MeCN in water (10 mmol/L NH₄HCO₃) to provide N-(5-(1,1-dioxidoisothiazolidin-2-yl)-8-(methylamino)-2,7-naphthyridin-3-yl)cyclopropanecarboxamide as a white solid (4.5 mg, 10%). LCMS (ESI) m/z 362.1, [M+H]⁺. ¹H NMR (400 MHz, DMSO-d₆) δ 11.01 (s, 1H), 9.34 (s, 1H), 8.38 (s, 1H), 8.14 (q, J=4.4 Hz, 1H), 8.04 (s, 1H), 3.65 (t, J=6.6 Hz, 2H), 3.45 (t, J=7.4 Hz, 2H), 2.99 (d, J=4.4 Hz, 3H), 2.50-2.42 (m, 2H), 2.12-2.02 (m, 1H), 0.92-0.79 (m, 4H).

### Example 59: Synthesis of N-(8-(methylamino)-5-(1-oxo-1,3-dihydro-2H-pyrrolo[3,4-c]pyridin-2-yl)-2,7-naphthyridin-3-yl)cyclopropanecarboxamide

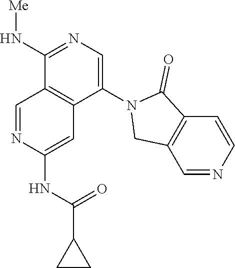 [SMILES: CNc1ncc(N2Cc3cnccc3C2=O)c2cc(NC(=O)C3CC3)ncc12]

To a solution of N-(5-bromo-8-(methylamino)-2,7-naphthyridin-3-yl)cyclopropanecarboxamide (40 mg; 0.12 mmol; 1.00 eq.) and 2,3-dihydro-1H-pyrrolo[3,4-c]pyridin-1-one (51 mg; 0.18 mmol; 1.50 eq.) in dioxane (6 mL) were added Cs₂CO₃ (81.5 mg; 0.25 mmol; 2.00 eq.) and Ephos Pd G₄ (CAS: 2132978-44-8) (11.4 mg; 0.012 mmol; 0.10 eq.), Ephos (CAS: 2118959-55-8) (6.6 mg; 0.012 mmol; 0.10 eq.). After stirring for 12 h at 120° C. under nitrogen atmosphere. The reaction was monitored by LCMS. The residue was purified by flash chromatography on silica gel column using MeOH/CH₂Cl₂ (2-6%) as eluent to provide N-(8-(methylamino)-5-(1-oxo-1,3-dihydro-2H-pyrrolo[3,4-c]pyridin-2-yl)-2,7-naphthyridin-3-yl)cyclopropanecarboxamide as a yellow solid (50 mg, crude). The residue was purified by flash chromatography on pre-packed C18 column using 20%-60% of MeCN in water (10 mmol/L NH₄HCO₃) to provide N-(8-(methylamino)-5-(1-oxo-1,3-dihydro-2H-pyrrolo[3,4-c]pyridin-2-yl)-2,7-naphthyridin-3-yl)cyclopropanecarboxamide as a white solid (43.9 mg, 91%). LCMS (ESI) m/z 375.1, [M+H]⁺. ¹H NMR (400 MHz, DMSO-d₆) δ 11.02 (s, 1H), 9.40 (s, 1H), 9.03 (s, 1H), 8.86 (d, J=5.2 Hz, 1H), 8.19-8.14 (m, 1H), 8.12 (s, 1H), 7.95 (s, 1H), 7.79 (d, J=5.2 Hz, 1H), 4.91 (s, 2H), 3.02 (d, J=4.4 Hz, 3H), 2.04-1.96 (m, 1H), 0.79-0.71 (m, 4H).

### Example 60: Synthesis of N-(8-(methylamino)-5-(4-oxopyridin-1(4H)-yl)-2,7-naphthyridin-3-yl)cyclopropanecarboxamide

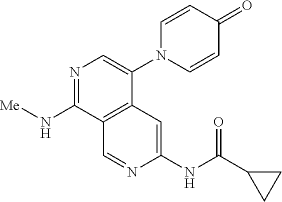 [SMILES: CNc1ncc(-n2ccc(=O)cc2)c2cc(NC(=O)C3CC3)ncc12]

To a solution of N-(5-bromo-8-(methylamino)-2,7-naphthyridin-3-yl)cyclopropanecarboxamide (100 mg; 0.311 mmol; 1.00 eq.) and pyridin-4-ol (177.9 mg; 1.871 mmol; 6.00 eq.) in DMSO (5 mL) were added dimethylglycine (28.9 mg; 0.280 mmol; 0.90 eq.), Cs₂CO₃ (203.9 mg; 0.626 mmol; 2.01 eq.) and CuI (29.6 mg; 0.155 mmol; 0.50 eq.). After stirring for 18 h at 120 DC under a nitrogen atmosphere. The desired product was observed via LCMS. The resulting mixture was purified by flash chromatography on pre-packed C18 column using 10-30% of MeCN in water (10 mmol/L NH₄HCO₃) as eluent to provide N-(8-(methylamino)-5-(4-oxopyridin-1(4H)-yl)-2,7-naphthyridin-3-yl)cyclopropanecarboxamide as a yellow solid (13.1 mg, 12%), LCMS (ESI) m/z 336.1, [M+H]⁺. ¹H NMR (400 MHz, DMSO-d₆) δ 11.11 (s, 1H), 9.42 (s, 1H), 8.36-8.25 (m, 1H), 8.14 (s, 1H), 7.95 (s, 1H), 7.68 (d, J=7.6 Hz, 2H), 6.21 (d, J=7.6 Hz, 2H), 2.99 (d, J=4.4 Hz, 3H), 2.09-2.00 (m, 1H), 0.90-0.70 (m, 4H).

### Example 61 and Example 62: Synthesis of N-(8-(methylamino)-5-(2-oxopyridin-1(2H)-yl)-2,7-naphthyridin-3-yl)cyclopropanecarboxamide and N-(8-(methylamino)-5-(pyridin-2-yloxy)-2,7-naphthyridin-3-yl)cyclopropanecarboxamide

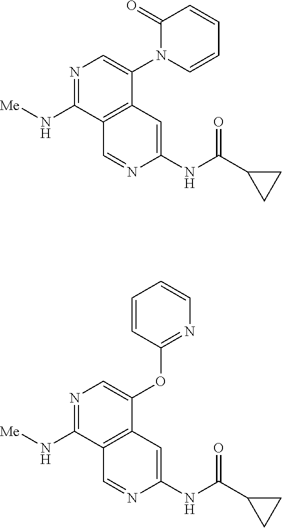 [SMILES: CNc1ncc(-n2ccccc2=O)c2cc(NC(=O)C3CC3)ncc12 ; CNc1ncc(Oc2ccccn2)c2cc(NC(=O)C3CC3)ncc12]

To a solution of N-(5-bromo-8-(methylamino)-2,7-naphthyridin-3-yl)cyclopropanecarboxamide (100 mg; 0.311 mmol; 1.00 eq.) and pyridin-2-ol (178 mg; 1.872 mmol; 6.00 eq.) in DMSO (3 mL) were added Cut (29.5 mg; 0.155 mmol; 0.50 eq.) and Cs₂CO₃ (203.9 mg; 0.626 mmol; 2.01 eq.), dimethylglycine (29 mg; 0.281 mmol; 0.90 eq.). After stirring for 3 days at 120° C. under a nitrogen atmosphere, the reaction was two peaks with desired product mass (17% & 21%). The resulting mixture was purified by flash chromatography on pre-packed C18 column using 10-30% of MeCN in water (10 mmol/L NH₄HCO₃) as eluent to provide 8 mg of the less polar peak and 20 mg of the more polar peak. Then the less peak was purified by reverse phase preparative HPLC (Prep-C18, XBridge Prep Phenyl OBD Column, 19×250 mm, water; gradient elution of 18-20% MeCN in water over a 10 min period, where both water and MeCN contain 10 mmol/L NH₄HCO₃, flow rate: 25 mL/min, detector UV wavelength: 254 nm) to provide N-(8-(methylamino)-5-(2-oxopyridin-1(2H)-yl)-2,7-naphthyridin-3-yl)cyclopropanecarboxamide as a white solid (3.4 mg, 3%). The more polar peak was purified by reverse phase preparative HPLC (Prep-C18, XBridge Shield RP18 OBD Column, 30×150 mm, waters; gradient elution of 30-40% MeCN in water over a 8 min period, where both water and MeCN contain 10 mmol/L NH₄HCO₃, flow rate: 60 mL/min, detector UV wavelength: 254 nm) to provide N-(8-(methylamino)-5-(pyridin-2-yloxy)-2,7-naphthyridin-3-yl)cyclopropanecarboxamide as a white solid (6.5 mg, 6%). LCMS (ESI) m/z 336.1, [M+H]⁺. ¹H NMR for N-(8-(methylamino)-5-(2-oxopyridin-1(2H)-yl)-2,7-naphthyridin-3-yl)cyclopropanecarboxamide: NMR (400 MHZ, DMSO-d₆) δ 11.01 (s, 1H), 9.40 (s, 1H), 8.28-8.16 (m, 1H), 7.94 (s, 1H), 7.77 (s, 1H), 7.65-7.54 (m, 2H), 6.51 (d, J=9.2 Hz, 1H), 6.34 (t, J=6.6 Hz, 1H), 3.02 (d, 4.4 Hz, 3H), 2.09-1.98 (m, 1H), 0.85-0.72 (m, 4H). HNMR for N-(8-(methylamino)-5-(pyridin-2-yloxy)-2,7-naphthyridin-3-yl)cyclopropanecarboxamide: ¹H NMR (400 MHz, DMSO-d₆) δ 10.97 (s, 1H), 9.37 (s, 1H), 8.08 (s, 1H), 8.03 (dd, J=4.8, 1.2 Hz, 1H), 7.91-7.80 (m, 3H), 7.15-7.06 (m, 2H), 2.99 (d, J=4.4 Hz, 3H), 2.09-2.00 (m, 1H), 0.85 0.74 (m, 4H).

### Example 63: Synthesis of N-(8-(methylamino)-5-(thiazol-2-yl)-2,7-naphthyridin-3-yl)cyclopropanecarboxamide

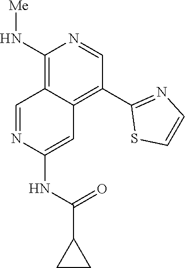 [SMILES: CNc1ncc(-c2nccs2)c2cc(NC(=O)C3CC3)ncc12]

To a stirring mixture of N-(5-bromo-8-(methylamino)-2,7-naphthyridin-3-yl)cyclopropanecarboxamide (50 mg; 0.156 mmol; 1.00 eq.) in dioxane (2 mL) were added Pd(PPh₃)₂Cl₂(21.9 mg; 0.031 mmol; 0.20 eq.), CuI (4.5 mg; 0.024 mmol; 0.15 eq.) and 2-(tributylstannyl)thiazole (116.5 mg; 0.311 mmol; 2.00 eq.) at room temperature under N₂, atmosphere. The reaction was stirred at 100° C. for 16 h. The desired product was observed via LCMS. The residue was purified by flash chromatography on silica gel column using MeOH/CH₂C₁, (2-8%) as eluent to provide N-(8-(methylamino)-5-(thiazol-2-yl)-2,7-naphthyridin-3-yl)cyclopropanecarboxamide (30 mg, crude). The residue was purified by flash chromatography on pre-packed C18 column using 15-70% of MeCN in water (10 mmol/L NH₄HCO₃) to provide N-(8-(methylamino)-5-(thiazol-2-yl)-2,7-naphthyridin-3-yl)cyclopropanecarboxamide as a yellow solid (17.7 mg, 34%). LCMS (ESI) m/z 326.1, [M+H]⁺. ¹H NMR (400 DMSO-d₆) δ 11.00 (s, 1H), 9.40 (s, 1H), 9.29 (s, 1H), 8.46 (s, 1H), 8.38-8.33 (m, 1H), 7.96 (d, J=3.2 Hz, 1H), 7.73 (d, J=3.2 Hz, 1H), 3.04 (d, J=4.4 Hz, 3H), 2.11-2.02 (m, 1H), 0.88-0.78 (m, 4H).

### Example 64: Synthesis of N-(8-(methylamino)-5-(1-(2,2,2-trifluoroethyl)-1H-pyrazol-3-yl)-2,7-naphthyridin-3-yl)cyclopropanecarboxamide

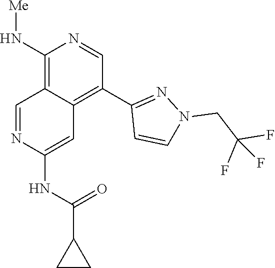 [SMILES: CNc1ncc(-c2ccn(CC(F)(F)F)n2)c2cc(NC(=O)C3CC3)ncc12]

### Step 1: 3-(4,4,5,5-tetramethyl-1,3,2-dioxaborolan-2-yl)-1-(2,2,2-trifluoroethyl)-1H-pyrazole

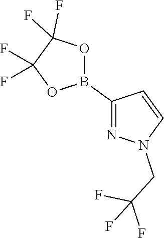 [SMILES: FC(F)(F)Cn1ccc(B2OC(F)(F)C(F)(F)O2)n1]

To a stirred solution of 5-(4,4,5,5-tetramethyl-1,3,2-dioxaborolan-2-yl)-1H-pyrazole (500 mg; 2.57 mmol; 1.00 eq.) in DMF (7 mL) was added NaH (60%) (256 mg; 6.40 mmol; 2.50 eq.) at 0° C. and stirred for 0.5 h at room temperature. To this resulting mixture was added 2,2,2-trifluoroethyl trifluoromethanesulfonate (597 mg; 2.57 mmol; 1.00 eq.) and stirred for 2 h at room temperature under nitrogen atmosphere. There were two peaks with the desired product mass. The reaction mixture was quenched by a sat. NH₄Cl solution (0.2 mL) at 0° C. The resulting mixture was diluted with EtOAc (100 mL) and washed with a sat. Na₄Cl solution (3×10 mL). The organic phase was concentrated under reduced pressure to provide 3-(4,4,5,5-tetramethyl-1,3,2-dioxaborolan-2-yl)-1-(2,2,2-trifluoroethyl)-1H-pyrazole as a yellow solid (507 mg, crude). The crude product was used in the next step directly without further purification. LCMS (ESI) m/z 277.1, [M+H]⁺.

### Step 2: N-(8-(methylamino)-5-(1-(2,2,2-trifluoroethyl-1H-pyrazol-3-yl)-2,7-naphthyridin-3-yl)cyclopropanecarboxamide

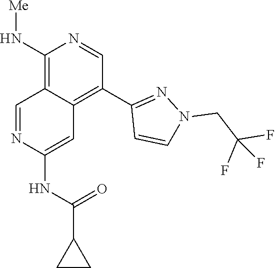 [SMILES: CNc1ncc(-c2ccn(CC(F)(F)F)n2)c2cc(NC(=O)C3CC3)ncc12]

A mixture of N-(5-bromo-8-(methylamino)-2,7-naphthyridin-3-yl)cyclopropanecarboxamide (50 mg; 0.156 mmol; 1.00 eq.), 3-(4,4,5,5-tetramethyl-1,3,2-dioxaborolan-2-yl)-1-(2,2,2-trifluoroethyl)-1H-pyrazole (129 mg; 0.46 mmol; 3.00 eq.), Pd(PPh₃)₄ (18 mg; 0.015 mmol; 0.01 eq.), Na₂CO₃ (50 mg; 0.47 mmol; 3.00 eq.) in DME/water (5:1, 0.4 mL, deoxygenated prior to use) was stirred for 3 h at 100° C. under nitrogen atmosphere. Upon completion, the resulting mixture was concentrated under reduced pressure. The residue was purified by flash chromatography on silica gel column using MeOH/CH₂Cl₂ (2-6%) petroleum ether as eluent to provide N-(8-(methyl amino)-5-(1-(2,2,2-trifluoroethyl)-1H-pyrazol-3-yl)-2,7-naphthyridin-3-yl)cyclopropanecarboxamide (40 mg, crude). The residue was purified by flash chromatography on pre-packed C18 column using 20%-60% of MeCN in water (10 mmol/L, NH₄HCO₃) to provide N-(8-(methylamino)-5-(1-(2,2,2-trifluoroethyl)-1H-pyrazol-3-yl)-2,7-naphthyridin-3-yl)cyclopropanecarboxamide as a white solid (30.6 mg, 50%). LCMS (ESI) m/z 391.1, [M+H]⁺. ¹H NMR (400 MHz, DMSO-d₆) δ 10.90 (s, 1H), 9.37 (s, 1H), 8.83 (s, 1H), 8.20 (s, 8.06-8.00 (m, 1H), 7.96 (d, J=2.0 Hz, 1H), 6.62 (d, J=2.0 Hz, 1H), 5.17 (q, J=9.2 Hz, 2H), 3.01 (d, J=4.4 Hz, 3H), 2.08-2.00 (m, 1H), 0.86-0.78 (m, 4H).

### Example 65: Synthesis of N-(8-(methylamino)-5-(thiazol-4-yl)-2,7-naphthyridin-3-yl)cyclopropanecarboxamide

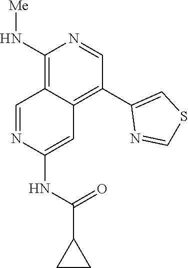 [SMILES: CNc1ncc(-c2cscn2)c2cc(NC(=O)C3CC3)ncc12]

A mixture of 4-(tributylstannyl)thiazole (175 mg; 0.468 mmol; 3.00 eq.), N-(5-bromo-8-(methylamino)-2,7-naphthyridin-3-yl)cyclopropanecarboxamide (50 mg; 0.156 mmol; 1.00 eq.), Pd(PPh₃)₂Cl₂ (13.5 mg; 0.019 mmol, 0.12 eq.) and Cid (2.7 mg; 0.014 mmol; 0.09 eq.) in dioxane (2 mL, deoxygenated prior to use) was stirred at 100° C. for 8 h under nitrogen atmosphere. The desired product was detected via. LCMS. The reaction was concentrated under reduced pressure and purified by flash chromatography on silica gel column using MeOH/CH₂Cl₂ (1-6%) to afford the crude product. The crude product was purified by flash chromatography on pre-packed C18 column using 10-37% of MeCN in water (10 mmol/L NH₄HCO₃) to afford N-(8-(methylamino)-5-(thiazol-4-yl)-2,7-naphthyridin-3-yl)cyclopropanecarboxamide as a light-yellow solid (8.3 mg, 16%). LCMS (ESI) m/z 326.1, [M+H]⁺. ¹H NMR (400 MHz, DMSO-d₆) δ 10.93 (5, 1H), 9.38 (s, 1H), 9.26 (d, J=2.0 Hz, 1H), 8.72 (s, 11=1), 8.25 (s, 1H), 8.10-8.04 (m, 1H), 7.78 (d, J=2.0 Hz, 1H), 3.02 (d, J=4.4 Hz, 3H), 2.07-2.00 (m, 1H), 0.85-0.78 (m, 4H).

### Example 66: Synthesis of N-(8-(methylamino)-5-(5-methylthiazol-2-yl)-2,7-naphthyridin-3-yl)cyclopropanecarboxamide

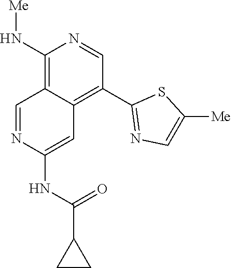 [SMILES: CNc1ncc(-c2ncc(C)s2)c2cc(NC(=O)C3CC3)ncc12]

A mixture of 1-(5-bromo-8-(methylamino)-2,7-naphthyridin-3-yl)cyclopropanecarboxamide (40.0 mg; 0.125 mmol, 1.00 eq.) and 5-methyl-2-(tributylstannyl)thiazole (241 mg; 0.625 mmol; 5.00 eq.), CuI (4.7 mg; 0.025 mmol; 0.2.0 eq.), Pd(PPh₃)₂Cl₂ (34.9 mg; 0.050 mmol; 0.40 eq.) in dioxane (2 mL, deoxygenated prior to use) was stirred for overnight at 100° C. under nitrogen atmosphere. The reaction was monitored by LCMS. The resulting mixture was concentrated under reduced pressure. The residue was purified by flash chromatography on silica gel column using MeOH/CH₂Cl₂ (2-10%) as eluent to provide the desired crude product. The crude product was purified by flash chromatography on pre-packed C18 column using 20-50% of MeCN in water (10 mmol/L NH₄HCO₃) as eluent to provide N-(8-(methylamino)-5-(5-methylthiazol-2-yl)-2,7-naphthyridin-3-yl)cyclopropanecarboxamide as a yellow solid (23.9 mg, 56%). LCMS (ESI) m/z 340.1, [M+H]⁺. ¹H NMR (400 MHz, DMSO-d₆) δ 10.98 (s, 1H), 9.39 (s, 1H), 9.24 (s, 1H), 8.37 (s, 1H), 8.33-8.28 (m, 1H), 7.63 (s, 1H), 3.03 (d, J=4.4 Hz, 3H), 2.50 (s, 3H), 2.09-2.01 (m, 1H), 0.87-0.78 (m, 4H).

### Example 67: Synthesis of N-(5-(3,6-dihydro-2H-pyran-4-yl)-8-(methylamino)-2,7-naphthyridin-3-yl)cyclopropanecarboxamide

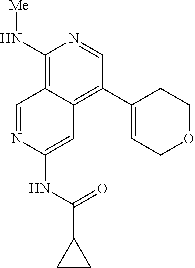 [SMILES: CNc1ncc(C2=CCOCC2)c2cc(NC(=O)C3CC3)ncc12]

A mixture of of N-(5-bromo-8-(methylamino)-2,7-naphthyridin-3-yl)cyclopropanecarboxamide (50 mg; 0.156 mmol; 1.00 eq.) and 2-(3,6-dihydro-2H-pyran-4-yl)-4,4,5,5-tetramethyl-1,3,2-dioxaborolane (98 mg; 0.466 mmol/L; 3.00 eq.) in DME/water (5:1, 1.2 mL, deoxygenated prior to use) was added Pd(PPh₃)₄ (18.1 mg; 0.015 mmol/L; 0.10 eq.) and Na₂CO₃ (33 mg; 0.311 mmol/L; 2.00 eq.) was stirred at 100° C. for 2 h under nitrogen atmosphere. The desired product was observed via LCMS. The solvent was concentrated under vacuum. The residue was purified by flash chromatography on silica gel column using MeOH in CH₂Cl₂ (2-10%) as eluent to provide the desired crude product. The crude product was purified by flash chromatography on pre-packed C18 column using 20-50% of MeCN in water (10 mmol/L NH₄HCO₃) as eluent to provide N-(5-(3,6-dihydro-2H-pyran-4-yl)-8-(methylamino)-2,7-naphthyridin-3-yl)cyclopropanecarboxamide as a white solid (35 mg, 69%). LCMS (ESI) m/z 325.2, [M+H]⁺. ¹H NMR (400 MHz, DMSO-d₆) δ 10.95 (s, 1H), 9.32 (s, 1H), 8.36 (s, 1H), 7.93-7.85 (m, 1H), 7.83 (s, 1H), 5.79 (s, 1H), 4.23 (d, 2.4 Hz, 2H), 3.85 (t, J=5.2 Hz, 2H), 2.97 (d, J=4.4 Hz, 3H), 2.40-2.31 (m, 2H), 2.11-2.00 (m, 1H), 0.91-0.77 (m, 4H).

### Example 68: Synthesis of N-(8-(methylamino)-5-(tetrahydro-2 pyran-4-yl)-2,7-naphthyridin-3-yl)cyclopropanecarboxamide

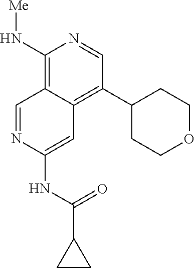 [SMILES: CNc1ncc(C2CCOCC2)c2cc(NC(=O)C3CC3)ncc12]

To a solution of N-(5-(3,6-dihydro-2H-pyran-4-yl)-8-(methylamino)-2,7-naphthyridin-3-yl)cyclopropanecarboxamide (Example 67) (30 mg; 0.092 mmol; 1.00 eq.) in MeOH (30 mL) was added 10% Pd/C (30 mg; 100% w/w) under nitrogen atmosphere. The mixture was hydrogenated at 40° C. for 4 hours under hydrogen atmosphere (2 atm). After filtration, the filtrate was concentrated under reduced pressure. The residue was purified by flash chromatography on silica gel column using 2-9% of MeOH in CH₂Cl₂ as eluent to provide N-(8-(methylamino)-5-(tetrahydro-2H-pyran-4-yl)-2,7-naphthyridin-3-yl)cyclopropanecarboxamide (15 mg, crude). The crude product was purified by flash chromatography on pre-packed C18 column using 15-50% of MeCN in water (10 mmol/L NH₄HCO₃) to provide N-(8-(methylamino)-5-(tetrahydro-2H-pyran-4-yl)-2,7-naphthyridin-3-yl)cyclopropanecarboxamide as a white solid (4.1 mg, 13%). LCMS (ESI) m/z 327.2, [M+H]⁺. ¹H NMR (400 MHz, DMSO-d₆) δ 10.97 (s, 9.32 (s, 1H), 8.45 (s, 1H), 7.90 (s, 1H), 7.75-7.70 (m, 1H), 4.02-3.94 (m, 2H), 3.55-3.45 (m, 2H), 3.06-2.97 (m, 1H), 2.94 (d, J=4.4 Hz, 3H), 2.10-2.02 (m, 1H), 1.81-1.68 (m, 4H), 0.91-0.78 (m, 4H).

### Example 69: Synthesis of N-(5-(cyclohex-1-en-1-yl)-8-(methylamino)-2,7-naphthyridin-3-yl)cyclopropanecarboxamide

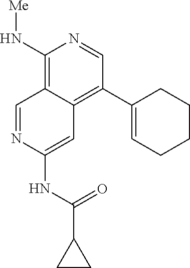 [SMILES: CNc1ncc(C2=CCCCC2)c2cc(NC(=O)C3CC3)ncc12]

A mixture of N-(5-bromo-8-(methylamino)-2,7-naphthyridin-3-yl)cyclopropanecarboxamide (50 mg; 0.156 mmol; 1.00 eq.) in a mixture solvent of DME/water (5:1, 2.4 mL) was added. Pd(PPh₃)₄ (18.0 mg; 0.016 mmol; 0.10 eq.), Na₂CO₃ (49.5 mg; 0.467 mmol; 3.00 eq.) and 2-(cyclohex-1-en-1-yl)-4,4,5,5-tetramethyl-1,3,2-dioxaborolane (97.5 mg; 0.469 mmol; 3.00 eq.) at room temperature. The reaction was stirred at 100° C. for 3 h under nitrogen atmosphere. The desired product was observed via LCMS. The resulting mixture was concentrated under vacuum. The residue was purified by flash chromatography on silica gel column using MeOH/CH₂Cl₂ (1-10%) as eluent to provide N-(5-(cyclohex-1-en-1-yl)-8-(methylamino)-2,7-naphthyridin-3-yl)cyclopropanecarboxamide (65 mg, crude). The crude product was purified by flash chromatography on pre-packed C18 column using 15%-60% of MeCN in water (10 mmol/L NH₄HCO₃) to provide N-(5-(cyclohex-1-en-1-yl)-8-(methylamino)-2,7-naphthyridin-3-yl)cyclopropanecarboxamide as a white solid (6.1 mg, 12%). LCMS (ESI) m/z 323.2, [M+H]⁺. ¹H NMR. (400 MHz, Methanol-d₄) δ 9.16 (s, 1H), 8.36 (s, 1.14), 7.70 (s, 1H), 5.78-5.71 (m, 1H), 3.06 (s, 3H), 2.34-2.22 (m, 4H), 1.97-1.90 (m, 1H), 1.89-1.75 (m, 4H), 1.06-1.01 (m, 2H), 0.95-0.89 (m, 2H).

### Example 70: Synthesis of N-(5-cyclohexyl-8-(methylamino)-2,7-naphthyridin-3-yl)cyclopropanecarboxamide

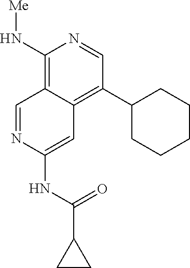 [SMILES: CNc1ncc(C2CCCCC2)c2cc(NC(=O)C3CC3)ncc12]

To a mixture of N-(5-(cyclohex-1-en-1-yl)-8-(methylamino)-2,7-naphthyridin-3-yl)cyclopropanecarboxamide (Example 69) (60 mg; 0.186 mmol; 1.00 eq.) in MeOH (30 mL) was added 10% Pd/C (60 mg; 100% w/w) under nitrogen atmosphere. The mixture was hydrogenated at 40° C. for 4 hours under hydrogen atmosphere (2 atm). The reaction was filtered through a Celite pad and concentrated under reduced pressure. The residue was purified by flash chromatography on silica gel column using MeOH/CH₂Cl₂ (1-9%) as eluent to provide N-(5-cyclohexyl-8-(methylamino)-2,7-naphthyridin-3-yl)cyclopropanecarboxamide (20 mg, crude). The crude product was purified by flash chromatography on pre-packed C18 column using 15%-70% of MeCN in water (10 mmol/L NH₃HCO₃) to provide 1-(5-cyclohexyl-8-(methylamino)-2,7-naphthyridin-3-yl)cyclopropanecarboxamide as a white solid (4.8 mg, 8%). LCMS (ESI) m/z 325.2, [M+H]⁺. ¹H NMR (400 MHz, DMSO-d₆) δ 10.93 (s, 1H), 9.30 (s, 1H), 8.44 (s, 1H), 7.87 (s, 1H), 7.68-7.63 (m, 1H), 2.93 (d, J=4.4 Hz, 3H), 2.78-2.69 (m, 1H), 2.10-2.02 (m, 1H), 1.89-1.71 (m, 51-1), 1.51-1.20 (m, 5H), 0.91-0.78 (m, 4H).

### Example 71: Synthesis N-(8-(methylamino)-5-vinyl-2,7-naphthyridin-3-yl)cyclopropanecarboxamide

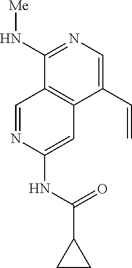 [SMILES: C=Cc1cnc(NC)c2cnc(NC(=O)C3CC3)cc12]

To a solution of N-(5-bromo-8-(methylamino)-2,7-naphthyridin-3-yl)cyclopropanecarboxamide (50 mg; 0.156 mmol; 1.00 eq.) in a mixture solvent of dioxane/water (5:1, 2.4 mL) were added Pd(DtBPF)Cl₂ (10.2 mg; 0.016 mmol; 0.10 eq.), K₃PO₄ (660.1 mg; 0.311 mmol; 2.00 eq.) and 4,4,5,5-tetramethyl-2-vinyl-1,3,2-dioxaborolane (72 mg; 0.467 mmol; 3.00 eq.) at room temperature. The reaction was stirred under nitrogen at 90° C. for 2 h under N₂ atmosphere. Upon completion, the resulting mixture was concentrated under vacuum. The residue was purified by flash chromatography on silica gel column using MeOH in CH₂Cl₂ (1-9%) as eluent to provide N-(8-(methylamino)-5-vinyl-2,7-naphthyridin-3-yl)cyclopropanecarboxamide (50 mg, crude). The crude product was purified by flash chromatography on pre-packed C18 column using 10%-70% of MeCN in water (10 mmol/L NH₄HCO₃) to provide N-(8-(methylamino)-5-vinyl-2,7-naphthyridin-3-yl)cyclopropanecarboxamide as a yellow solid (5.2 mg, 12%). LCMS (ESI) m/z 269.1, [M+H]⁺. ¹H NMR (400 MHz, DMSO-d₆) δ 11.01 (s, 1H), 9.33 (s, 1H), 8.48 (s, 1H), 8.19 (s, 1H), 8.02-7.95 (m, 1H), 6.92 (dd, J=17.6, 11.2 Hz, 1H), 5.66 (dd, J=17.6, 1.2 Hz, 1H), 5.25 (dd, J=11.2, 1.2 Hz, 1H), 2.98 (d, J=4.4 Hz, 3H), 2.09-2.03 (m, 1H), 0.89-0.80 (m, 4H).

### Example 72: Synthesis of N-(5-ethyl-8-(methylamino)-2,7-naphthyridin-3-yl)cyclopropanecarboxamide

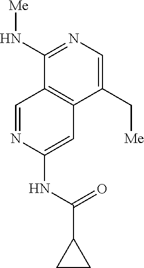 [SMILES: CCc1cnc(NC)c2cnc(NC(=O)C3CC3)cc12]

To a solution of N-(8-(methylamino)-5-vinyl-2,7-naphthyridin-3-yl)cyclopropanecarboxamide (Example 71) (43 mg; 0.160 mmol; 1.00 eq.) in MeOH (10 mL) was added 10% Pd/C (1.7 mg; 40% w/w) under nitrogen atmosphere. The mixture was hydrogenated at 40° C. for 2 hours under hydrogen atmosphere (2 atm). The reaction mixture was filtered through a Celite pad and concentrated under reduced pressure. The resulting mixture was concentrated under vacuum. The residue was purified by flash chromatography on silica gel column using MeOH in CH₂Cl₂ (1-9%) as eluent to provide N-(5-ethyl-8-(methylamino)-2,7-naphthyridin-3-yl)cyclopropanecarboxamide (35 mg, crude). The crude product was purified by flash chromatography on pre-packed C18 column using 10%-70% of MeCN in water (10 mmol/L NH₄HCO₃) to provide N-(5-ethyl-8-(methylamino)-2,7-naphthyridin-3-yl)cyclopropanecarboxamide as a white solid (20.8 mg, 48%). LCMS (ESI) m/z 271.1, [M+H]⁺. ¹H NMR (400 MHz, DMSO-d₆) δ 10.97 (s, 1H), 9.31 (s, 1H), 8.35 (s, 1H), 7.84 (s, 1H), 7.71-7.65 (m, 1H), 2.94 (d, 4.4 Hz, 3H), 2.70-2.63 (m, 21-1), 2.10-2.02 (m, 1H), 1.19 (t, J=7.6 Hz, 3H), 0.90-0.80 (m, 4H).

### Example 73: Synthesis of N-(8-(methylamino)-5-(prop-1-en-2-yl)-2,7-naphthyridin-3-yl)cyclopropanecarboxamide

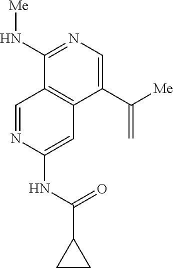 [SMILES: C=C(C)c1cnc(NC)c2cnc(NC(=O)C3CC3)cc12]

To a solution of N-(5-bromo-8-(methylamino)-2,7-naphthyridin-3-yl) cyclopropanecarboxamide (70 mg; 0.2.18 mmol; 1.00 eq.) and 4,4,5,5-tetramethyl-2-(prop-1-en-2-yl)-1,3,2-dioxaborolane (36 mg; 0.214 mmol; 1.00 eq.) in a mixture solvent of 1,4-dioxane/water (7:1, 2.4 were added Pd(DtBPF)Cl₂ (14 mg; 0.021 mmol; 0.10 eq.) and K₃PO₄ (91 mg; 0.429 mmol; 2.00 eq.). The mixture was stirred for 2 h at 90° C. under nitrogen atmosphere. The mixture was concentrated under vacuum. The residue was purified by flash chromatography on silica gel eluting using methanol in dichloromethane (5-10%) as eluent to afford the crude desired product. The crude product was further purified by flash chromatography on pre-packed C18 column using 20-50% of MeCN in water (10 mmol/L NH₄HCO₃) to provide N-(8-(methylamino)-5-(prop-1-en-2-yl)-2,7-naphthyridin-3-yl)cyclopropanecarboxamide as a white solid (5.0 mg) for delivery. LCMS (ESI) m/z 283.1, [M+H]⁺. ¹H NMR (400 MHz, DMSO-d₆) δ 10.94 (s, 1H), 9.32 (s, 1H), 8.39 (s, 1H), 7.86 (s, 1H), 7.85-7.82 (m, 1H), 5.31-5.28 (m, 1H), 5.00-4.97 (m, 1H), 2.96 (d, J=4.4 Hz, 3H), 2.08 (s, 3H), 2.06-2.00 (m, 1H), 0.88-0.78 (m, 4H)

### Example 74: Synthesis of 1%-(5-isopropyl-8-(methylamino)-2,7-naphthyridin-3-yl)cyclopropanecarboxamide

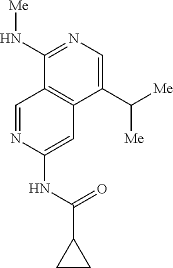 [SMILES: CNc1ncc(C(C)C)c2cc(NC(=O)C3CC3)ncc12]

To a stirring mixture of N-(8-(methylamino)-5-(prop-1-en-2-yl)-2,7-naphthyridin-3-yl)cyclopropanecarboxamide (Example 73) (60 mg; 0.2.13 mmol; 1.00 eq.) in MeOH (10 mL) was added 10% Pd/C (60 mg; 100% w/w). The reaction was stirred at 40° C. for 12 h under hydrogen atmosphere (2 atm). The desired product was observed via LCMS. The reaction was concentrated under reduced pressure and purified by flash chromatography on silica gel column using MeOH/CH₂Cl₂ (2-8%) to afford the desired crude product. The crude was further purified by reverse phase preparative HPLC (Prep-C18, 5 μM OBD column, 19×250 mm, water; gradient elution of 35-48% MeCN in water over 8 min, where both water and MeCN contain 10 mmol/L NH₄HCO₃, flow rate: 60 mL/min, detector UV wavelength: 254 nm) to afford N-(5-isopropyl-8-(methylamino)-2,7-naphthyridin-3-yl)cyclopropanecarboxamide as a white solid (11.4 mg, 18%). LCMS (ESI) m/z 285.2, [M+]⁺. ¹H NMR (400 MHz, DMSO-d₆) δ 10.97 (s, 1H); 9.31 (s, 1H), 8.45 (s, 1H), 7.91 (s, 1H), 7.71-7.66 (m, 1H), 3.21-3.12 (m, 1H), 2.94 (d, J=4.4 Hz, 3H), 2.10-2.02 (m, 1H), 1.27 (d, J=6.8 Hz, 6H), 0.89-0.80 (m, 4H).

### Example 75: Synthesis of N-(5-(2,5-dihydrofuran-3-yl)-8-(methylamino)-2,7-naphthyridin-3-yl)cyclopropanecarboxamide

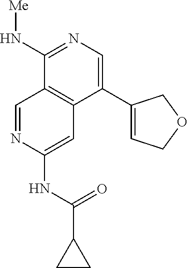 [SMILES: CNc1ncc(C2=CCOC2)c2cc(NC(=O)C3CC3)ncc12]

A mixture of N-(5-bromo-8-(methylamino)-2,7-naphthyridin-3-yl)cyclopropanecarboxamide (60 mg; 0.18 mmol; 1.00 eq.) and 2-(2,5-dihydrofuran-3-yl)-4,4,5,5-tetramethyl-1,3,2-dioxaborolane (44 mg; 0.22 mmol; 1.20 eq.), Pd(DtBPF)Cl₂. (12 mg; 0.01 mmol; 0.10 eq.), K₃PO₄(79 mg; 0.37 mmol; 2.00 eq.) in dioxane/water (5:1, 7.2 mL) was stirred for 2 h at 90° C. under nitrogen atmosphere. The reaction was monitored by LCMS. The resulting mixture was concentrated under reduced pressure. The residue was purified by flash chromatography on silica gel column using 2-6% of MeOH in CH₂Cl₂ as eluent to provide N-(5-(2,5-dihydrofuran-3-yl)-8-(methylamino)-2,7-naphthyridin-3-yl)cyclopropanecarboxamide (60 mg, crude). Then 10 mg of the crude product was purified by flash chromatography on pre-packed C18 column using 20%-60% of MeCN in water (10 mmol/L NH₄HCO₃) to provide N-(5-(2,5-dihydrofuran-3-yl)-8-(methylamino)-2,7-naphthyridin-3-yl)cyclopropanecarboxamide as a yellow solid (3.1 mg, 5%). LCMS (ESI) m/z 311.1, [M+H]⁺. ¹H NMR (400 MHz, DMSO-d₆) δ 11.06 (s, 1H), 9.35 (s, 1H), 8.68 (s, 1H), 8.08-8.03 (m, 1H), 7.94 (s, 1H), 6.21-6.19 (m, 1H), 4.94-4.89 (m, 2R), 4.82-4.77 (m, 2H), 2.98 (d, J=4.4 Hz, 3H), 2.11-2.03 (m, 1H), 0.90-0.79 (m, 4H).

### Example 76: Synthesis of N-(8-(methylamino)-5-(tetrahydrofuran-3-yl)-2,7-naphthyridin-3-yl)cyclopropanecarboxamide

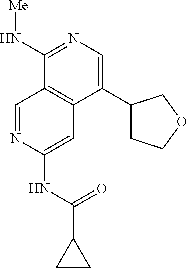 [SMILES: CNc1ncc(C2CCOC2)c2cc(NC(=O)C3CC3)ncc12]

To a solution of N-(5-(2,5-dihydrofuran-3-yl)-8-(methylamino)-2,7-naphthyridin-3-yl)cyclopropanecarboxamide (Example 75) (68 mg; 0.21 mmol; 1.00 eq.) in MeOH (100 mL) was added Pd/C (68 mg, 10% w/w) under nitrogen atmosphere. The mixture was hydrogenated at 30-40° C. under hydrogen atmosphere (2 atm) for 2 h. After filtration, the filtrate was concentrated under reduced pressure. The residue was purified by flash chromatography on pre-packed C18 column using 20%-60% of MeCN in water (10 mmol/l, NH₄HCO₃) to provide N-(8-(methylamino)-5-(tetrahydrofuran-3-yl)-2, 7-naphthyridin-3-yl)cyclopropanecarboxamide as a yellow solid (24.9 mg, 36%). LCMS (ESI) m/z 313.2, [M+H]⁺. ¹H NMR, (400 MHz, DMSO-d₆) 11.00 (s, 1H), 9.32 (s, 1H), 844 (s, 1H), 794 (s, 1H), 7.79-7.74 (m, 1H), 4.11-4.05 (m, 1H), 3.98-3.91 (m, 1H), 3.89-3.82 (m, 1H), 3.65-3.53 (m, 2H), 2.95 (d, J=4.4 Hz, 3H), 2.35-2.24 (m, 1H), 2.10-1.99 (m, 2H), 0.91-0.80 (m, 4H).

### Example 77: Synthesis of N-(5-(5,6-dihydro-2H-pyran-3-yl)-8-(methylamino)-2,7-naphthyridin-3-yl)cyclopropanecarboxamide

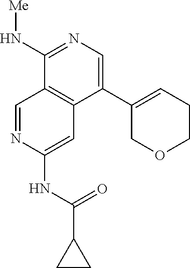 [SMILES: CNc1ncc(C2=CCCOC2)c2cc(NC(=O)C3CC3)ncc12]

A mixture of N-(5-bromo-8-(methylamino)-2,7-naphthyridin-3-yl)cyclopropanecarboxamide (40 mg; 0.125 mmol; 1.00 eq.) was added to 2-(5,6-dihydro-2/1-pyran-3-yl)-4,4,5,5-tetramethyl-1,3,2-dioxaborolane (52.0 mg; 0.248 mmol; 2.00 eq.), Pd(DtBPF)Cl₂ (8.12 mg; 0.012 mmol; 0.10 eq.) and K₃PO₄ (52.9 mg; 0.249 mmol; 2.00 eq.) in dioxane/water (5:1, 2.4 mL) was stirred at 90° C. for 2 h under nitrogen atmosphere. The desired product was observed via LCMS. The reaction mixture was concentrated in vacuo. The residue was purified by flash chromatography on silica gel column using MeOH/CH₂Cl₂(2-7%) to afford 65 mg of the crude desired product. The crude product was purified by flash chromatography on pre-packed C18 column using 10-40% of MeCN in water (10 mmol/L NH₄HCO₃) to afford N-(5-(5,6-dihydro-2H-pyran-3-yl)-8-(methylamino)-2,7-naphthyridin-3-yl)cyclopropanecarboxamide as a white solid (3.5 mg, 8%). LCMS (ESI) m/z 325.2, [M+H]⁺. ¹H NMR (400 MHz, DMSO-d₆) δ 10.93 (s, 1H), 9.30 (s, 1H), 8.36 (s, 1H), 7.90-7.85 (m, 1H), 7.81 (s, 1H), 5.86-5.83 (m, 1H), 4.18 (s, 2H), 3.83-3.77 (m, 2H), 2.96 (d, J=4.4 Hz, 3H), 2.31-2.22 (m, 2H), 2.09-2.01 (m, 1H), 0.89-0.79 (m, 4H).

### Example 78: Synthesis of N-(8-(methylamino)-5-(tetrahydro-2H-pyran-3-yl)-2,7-naphthyridin-3-yl)cyclopropanecarboxamide

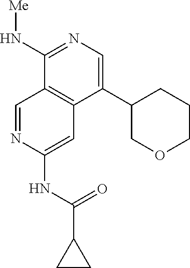 [SMILES: CNc1ncc(C2CCCOC2)c2cc(NC(=O)C3CC3)ncc12]

Under nitrogen atmosphere, To a stirring mixture of N-(5-(5,6-dihydro-2H-pyran-3-yl)-8-(methylamino)-2,7-naphthyridin-3-yl)cyclopropanecarboxamide (Example 77) (50 mg; 0.154 mmol; 1.00 eq.) in MeOH (50 mL) was added 10% Pd/C (50 mg; 100% w/w). The reaction was stirred at 40° C. overnight under hydrogen atmosphere (2 atm). The desired product was observed via LCMS. The reaction was concentrated under reduced pressure and purified by flash chromatography on silica gel column using MeOH/CH₂Cl₂. (2-8%) to afford 31.0 mg of the crude product, the crude was purified by reverse phase preparative HPLC (Prep-C18, 5 μM OBD column, 19×250 mm, water; gradient elution of 37% MeCN in water over a 8 min period, where both water and MeCN contain 0.1% formic acid, flow rate: 60 mL/min, detector UV wavelength: 254 nm) to afford N-(8-(methylamino)-5-(tetrahydro-2H-pyran-3-yl)-2,7-naphthyridin-3-yl)cyclopropanecarboxamide as a white solid (6.1 mg, 12%). LCMS (ESI) m/z 327.2, [M+H]⁺. ¹H NMR (400 MHz Methanol-d₄) δ 9.28 (s, 1H), 8.61 (s, 1H), 7.75 (s, 1H), 4.09-3.92 (m, 2H), 3.64-3.50 (m, 2H), 3.27-3.19 (m, 1H), 3.12 (s, 3H), 2.16-2.08 (m, 1H), 2.00-1.75 (m, 4H), 1.08-1.04 (m, 2H), 0.98-0.93 (m, 2H).

### Example 79: Synthesis of (E)-N-(5-((dihydrofuran-3(2H)-ylidene)methyl-8-(methylamino)-2,7-naphthyridin-3-yl)cyclopropanecarboxamide

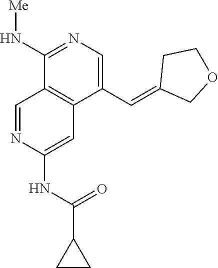 [SMILES: CNc1ncc(/C=C2\CCOC2)c2cc(NC(=O)C3CC3)ncc12]

A mixture of N-(5-(promo-8-(methylamino)-2,7-naphthyridin-3-yl)cyclopropanecarboxamide (60 mg; 0.187 mmol; 1.00 eq.), (E)-2-((dihydrofuran-3(2H)-ylidene)methyl)-4,4,5,5-tetramethyl-1,3,2-dioxaborolane (47.3 mg; 0.225 mmol; 1.21 eq.), Pd(DtBPF)Cl₂. (12.2 mg; 0.019 mmol; 0.10 eq.) and K₃PO₄ (79.7 mg; 0.375 mmol; 2.01 eq.) in 1,4-dioxane/water (5:1, 2.4 mL) was stirred for 2 h at 90° C. under nitrogen atmosphere. The desired product was observed via LCMS. The resulting mixture was concentrated under reduced pressure. The residue was purified by flash chromatography on silica gel column using MeOH/CH₂Cl₂ (2-10%) as eluent to provide the crude desired product. The crude product was further purified by reverse phase preparative HPLC (XBridge Shield RP18 OBD Column, 30×150 mm, waters; gradient elution of 30-40% MeCN in water over a 8 min period, where both water and MeCN contain 10 mmol/L NH₄HCO₃, flow rate: 60 mL/min, detector UV wavelength: 254 nm) to provide (E)-N-(5-((dihydrofuran-3(2H)-ylidene)methyl)-8-(methylamino)-2,7-naphthyridin-3-yl)cyclopropanecarboxamide as a yellow solid (50 mg, 82%). LCMS (ESI) m/z 325.2, [M+H]⁺. ¹H NMR (400 MHz, DMSO-d₆) 11.01 (s, 1H), 9.33 (s, 1H), 8.36 (s, 1H), 7.97-7.90 (m, 1H), 7.79 (s, 1H), 6.54 (s, 1H), 4.41-4.33 (m, 2H), 3.86-3.77 (m, 2H), 2.98 (d, J=4.4 Hz, 3H), 2.79-2.63 (m, 2H), 2.10-2.02 (m, 1H), 0.88-0.80 (m, 4H).

### Example 80: Synthesis of N-(8-(methylamino)-5-((tetrahydrofuran-3-yl)methyl)-2,7-naphthyridin-3-yl)cyclopropanecarboxamide

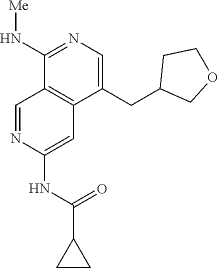 [SMILES: CNc1ncc(CC2CCOC2)c2cc(NC(=O)C3CC3)ncc12]

To a solution of (E)-N-(5-((dihydrofuran-3(2H)-ylidene)methyl)-8-(methylamino)-2,7-naphthyridin-3-yl)cyclopropanecarboxamide (Example 79) (40 mg; 0.123 mmol; 1.00 eq.) in MeOH (2.0 mL) was added 10% Pd/C (40 mg, 100% w/w). The mixture was hydrogenated at room temperature under hydrogen atmosphere (2 atm) for 2 h. The reaction was monitored via LCMS. After filtration, the filtrate was concentrated under reduced pressure to provide N-(8-(methylamino)-5-((tetrahydrofuran-3-yl)methyl)-2,7-naphthyridin-3-yl)cyclopropanecarboxamide as a white solid (60 mg, crude). The crude product was purified by flash chromatography on pre-packed. C18 column using 20-50% of MeCN in water (10 mmol/L NH₄HCO₃) as eluent to provide N-(8-(methylamino)-5-((tetrahydrofuran-3-yl)methyl)-2,7-naphthyridin-3-yl)cyclopropanecarboxamide as a white solid (15.6 mg, 38%). LCMS (ESI) m/z 327.2, [M+H]⁺. ¹H NMR (400 MHz, DMSO-d₆) δ 10.98 (s, 1H), 9.31 (s, 1H), 8.36 (s, 1H), 7.85 (s, 1H), 7.76-7.70 (m, 1H), 3.83-3.75 (m, 1H)), 3.70-3.57 (m, 2H)), 3.41-3.34 (m, 1H), 2.94 (d, J=4.4 Hz, 3H), 2.72-2.64 (m, 2H), 2.57-2.52 (m, 1H), 2.09-2.02 (m, 1H), 1.92-1.84 (m, 1H), 1.60-1.51 (m, 1H), 0.91-0.80 (m, 4H).

### Example 81: Synthesis of N-(8-(methylamino)-5-(2-methylthiazol-4-yl)-2,7-naphthyridin-3-yl)cyclopropanecarboxamide

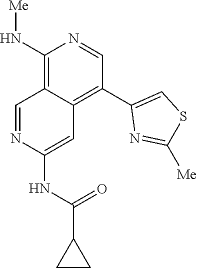 [SMILES: CNc1ncc(-c2csc(C)n2)c2cc(NC(=O)C3CC3)ncc12]

### Step 1: N-(8-(methylamino)-5-(4,4,5,5-tetramethyl-1,3,2-dioxaborolan-2-yl)-2,7-naphthyridin-3-yl)cyclopropanecarboxamide

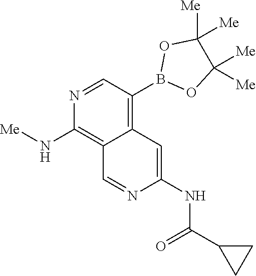 [SMILES: CNc1ncc(B2OC(C)(C)C(C)(C)O2)c2cc(NC(=O)C3CC3)ncc12]

A mixture of N-(5-bromo-8-(methylamino)-2,7-naphthyridin-3-yl)cyclopropanecarboxamide (500 mg; 1.557 mmol; 1.00 eq.) and 4,4,4′,4′,5,5,5′,5′-octamethyl-2,2′-bi(1,3,2-dioxaborolane) (3.96 g; 15.59 mmol; 10.00 (eq.), Pd(dppf)Cl₂ (128 mg; 0.175 mmol; 0.10 eq.), KOAc (306 mg; 3.118 mmol; 2.00 eq.) in 1,4-dioxane (24 mL) was stirred for 3 h at 100° C. under nitrogen atmosphere. The resulting mixture was concentrated under reduced pressure. The residue was dissolved in a mixture solvent of petroleum ether/EtOAc (10:1, 200 mL) and the precipitate was formed. The solids were collected by filtration and washed with a mixture solvent of petroleum ether/EtOAc(10:1, 20 mL). The solids were dried under reduced pressure to afford N-(8-(methylamino)-5-(4,4,5,5-tetramethyl-1,3,2-dioxaborolan-2-yl)-2,7-naphthyridin-3-yl)cyclopropanecarboxamide as a brown solid (916 mg, crude). LCMS (ESI) m/z 369.2, [M+H]⁺.

### Step N-(8-(methylamino)-5-(2-methylthiazol-4-yl)-2,7-naphthyridin-3-yl)cyclopropanecarboxamide

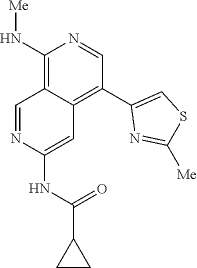 [SMILES: CNc1ncc(-c2csc(C)n2)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (100 mg; 0.272 mmol; 1.00 eq.), 4-bromo-2-methylthiazole (48.3 mg; 0.272 mmol; 1.00 eq.), Pd(DtBPF)Cl₂ (CAS: 95408-45-yl) (17.7 mg; 0.027 mmol; 0.10 eq.) and K₃PO₄. (115 mg; 0.544 mmol; 2.00 eq.) in dioxane/water (6:1, 2.9 mL) was stirred for 2 h at 90° C. under nitrogen atmosphere. The desired product was observed via LCMS. The resulting mixture was concentrated under reduced pressure. The residue was purified by flash chromatography on silica gel column using MeOH/CH₂Cl₂ (2-10%) as eluent to provide the crude product. The crude product was further purified by flash chromatography on pre-packed C18 column using 20-50% of MeCN in water (10 mmol/L, NH₄HCO₃) as eluent to provide N-(8-(methylamino)-5-(2-methylthiazol-4-yl)-2,7-naphthyridin-3-yl)cyclopropanecarboxamide as a white solid (20.7 mg, 21%), LCMS (ESI) m/z 340.1, [M+H]⁺. ¹H NMR (400 MHz, DMSO-d₆) δ 10.93 (s, 1H), 9.37 (s, 1H), 8.72 (s, 1H), 8.23 (s, 1H), 8.07-8.01 (m, 1H), 7.51 (s, 1H), 3.01 (d, J=4.4, 3H), 2.73 (s, 3H), 2.08-2.00 (m, 1H), 0.86-0.77 (m, 4H).

### Example 82: Synthesis of N-(5-(isothiazol-3-yl)-8-(methylamino)-2,7-naphthyridin-3-yl)cyclopropanecarboxamide

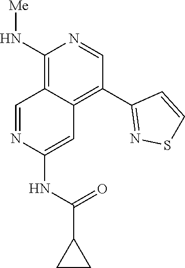 [SMILES: CNc1ncc(-c2ccsn2)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (Example 81, Step 1) (100 mg; 0.272 mmol; 1.00 eq.), 3-bromoisothiazole (44.5 mg; 0.272 mmol; 1.00 eq.), Pd(dppf)Cl₂. CH₂Cl₂ (22.1 mg; 0.027 mmol; 0.10 eq.) and K₃PO₄ (115 mg; 0.544 mmol; 2.00 eq.) in 1,4-dioxane/water (5:1, 3.0 mL) is was stirred for 2 h at 40° C. under nitrogen atmosphere. The desired product was observed via LCMS. The resulting mixture was concentrated under reduced pressure. The residue was purified by flash chromatography on silica gel column using NeOH/CH₂Cl₂. (2-10%) as eluent to provide the crude desired product. The crude product was further purified by flash chromatography on pre-packed C18 column using 20-50% of MeCN in water (10 mmol/L NH₄HCO₃) as eluent to provide N-(5-(isothiazol-3-yl)-8-(methylamino)-2,7-naphthyridin-3-yl)cyclopropanecarboxamide as a white solid (8.6 mg, 10%). LCMS (ESI) m/z 326.1, [M+H]⁺. ¹H NMR (400 MHz, DMSO-d₆) δ 10.95 (s, 1H), 9.39 (s, 1H), 9.17 (d, J=4.4 Hz, 1H), 9.14 (s, 1H), 8.42 (s, 1H), 8.23-8.17 (m, 1H), 7.72 (d, J=4.4 Hz, 1H), 3.03 (d, J=4.4 Hz, 3H), 2.08-2.01 (m, 1H), 0.86-0.78 (m, 4H).

### Examples 83 and 84

Each compound in Table 3 below was prepared using a similar procedure to prepare Example 82 using N-(8-(methylamino)-5-(4,4,5,5-tetramethyl-1,3,2-dioxaborolan-2-yl)-2,7-naphthyridin-3-yl)cyclopropanecarboxamide as the common intermediate and appropriate halogenated aromatic:

TABLE 3

| Example | Structure and Name | MS and ¹H NMR |
|---|---|---|
| Example 83 | 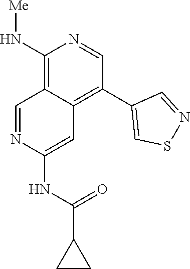 | LCMS (ESI) m/z 326.1, [M + H]⁺. ¹H NMR (400 MHz, DMSO-d₆) δ 11.01 (s, 1H), 9.40 (s, 1H), 9.08 (s, 1H), 8.74 (s, 1H), 8.37 (s, 1H), 8.13 (s, 1H), 8.12- 8.02 (m, 1H), 3.02 (d, J = 4.4 Hz, 3H), 2.10-2.00 (m, 1H), 0.90-0.74 (m, 4H). |
| | N-(5-(isothiazol-4-yl)-8- | |
| | (methylamino)-2,7- | |
| | naphthyridin-3- | |
| | yl)cyclopropanecarboxamide | |
| Example 84 | 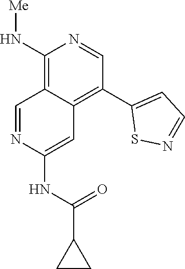 | LCMS (ESI) m/z 326.1, [M + H]⁺. ¹H NMR (400 MHz, DMSO-d₆) δ 11.09 (s, 1H), 9.41 (s, 1H), 8.66 (d, J = 1.6 Hz, 1H), 8.64 (s, 1H), 8.33-8.28 (m, 1H), 8.23 (s, 1H), 7.57 (d, J = 1.6 Hz, 1H), 3.03 (d, J = 4.4 Hz, 3H), 2.08-2.01 (m, 1H), 0.87- 0.78 (m, 4H). |
| | N-(5-(isothiazol-5-yl)-8- | |
| | (methylamino)-2,7- | |
| | naphthyridin-3- | |
| | yl)cyclopropanecarboxamide | |

### Example 85: Synthesis of N-(8-(methylamino)-5-(2-oxo-2,5-dihydrofuran-3-yl)-2,7-naphthyridin-3-yl)cyclopropanecarboxamide

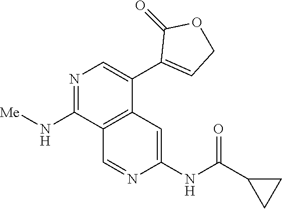 [SMILES: CNc1ncc(C2=CCOC2=O)c2cc(NC(=O)C3CC3)ncc12]

To a stirring mixture of solution of N-(8-(methylamino)-5-(4,4,5,5-tetramethyl-1,3,2-dioxaborolan-2-yl)-2,7-naphthyridin-3-yl)cyclopropanecarboxamide (Example 81, Step 1) (71 mg; 0.193 mmol; 1.00 eq.) in dioxane/water (5:1, 4.8 mL, deoxygenated prior to use) was added Pd(DtBPF)Cl₂ (12.6 mg; 0.019 mmol; 0.10 eq.), K₃PO₄ (810.8 mg; 0.385 mmol; 2.00 eq.) and 3-bromofuran-2(5H)-one (94.3 mg; 0.579 mmol; 3.00 eq.) at room temperature. The reaction was stirred at 90° C. for 2 h under nitrogen atmosphere. The desired product was Observed via LCMS. The resulting mixture was concentrated under vacuum. The residue was purified by flash chromatography on silica gel column using MeOH/CH₂Cl₂ (2-8%) as eluent to provide N-(8-(methylamino)-5-(2-oxo-2,5-dihydrofuran-3-yl)-2,7-naphthyridin-3-yl)cyclopropanecarboxamide as a yellow solid (20 mg, crude). The crude product was purified by flash chromatography on pre-packed C18 column using 20%-60% of MeCN in water (10 mmol/L NH₄HCO₃) to provide N-(8-(methylamino)-2,7-naphthyridin-3-yl)cyclopropanecarboxamide as a white solid (4.5 mg, 9%). Another residue was purified by flash chromatography on pre-packed C18 column using 20%-60% of MeCN in water (10 mmol/L NH₄HCO₃) to provide N-(8-(methylamino)-5-(2-oxo-2,5-di hydrofuran-3-yl)-2,7-naphthyridin-3-yl)cyclopropanecarboxamide as a white solid (2.1 mg, 3%). LCMS (ESI) m/z 325.1, [M+H]⁺. ¹H NMR (400 MHz, DMSO-d₆) δ 11.01 (s, 9.37 (s, 1H), 8.30 (s, 1H), 8.17-8.13 (m, 1H), 8.11 (s, 1H), 7.89-7.85 (m, 1H), 5.13-5.09 (m, 2H), 3.01 (d, J=4.4 Hz, 3H), 2.09-2.00 (m, 1H), 0.89-0.78 (m, 4H).

### Example 86: Synthesis of N-(8-(methylamino)-5-(2-oxotetrahydrofuran-3-yl)-2,7-naphthyridin-3-yl)cyclopropanecarboxamide

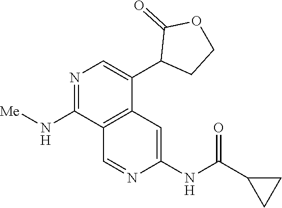 [SMILES: CNc1ncc(C2CCOC2=O)c2cc(NC(=O)C3CC3)ncc12]

To a solution of N-(8-(methylamino)-5-(2-oxo-2,5-di hydrofuran-3-yl)-2,7-naphthyridin-3-yl)cyclopropanecarboxamide (Example 85) (46 mg; 0.142 mmol; 1.00 eq.) in MeOH (20 mL) was added 10% Pd/C (46 mg; 100% w/w) under nitrogen atmosphere at room temperature. The mixture was hydrogenated at 40° C. for 16 h under hydrogen atmosphere (2 atm). After filtration, the filtrate was concentrated under reduced pressure. The residue was purified by flash chromatography on pre-packed C18 column using 20%-60% of MeCN in water (10 mmol/L-NH₄HCO₃) to provide N-(8-(methylamino)-5-(2-oxotetrahydrofuran-3-yl)-2,7-naphthyridin-3-yl)cyclopropanecarboxamide as a white solid (1.8 mg, 4%). LCMS (ESI) m/z 327.1, [M+H]⁺. ¹H NMR (400 MHz, DMSO-d₆) δ 11.00 (s, 1H), 9.35 (s, 1H), 8.23 (s, 1H), 7.95-7.91 (m, 1H), 7.91 (s, 1H), 4.54-4.37 (m, 2H), 4.18 (t, J=10.0 Hz, 1H), 2.96 (d, J=4.4 Hz, 3H), 2.68-2.56 (m, 1H), 2.46-2.35 (m, 1H), 2.11-2.02 (m, 1H), 0.90-0.80 (m, 4H).

### Example 87: Synthesis of N-(5-(1-(cyanomethyl)-1N-pyrazol-3-yl)-8-(methylamino)-2,7-naphthyridin-3-yl)cyclopropanecarboxamide

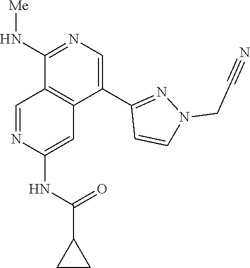 [SMILES: CNc1ncc(-c2ccn(CC#N)n2)c2cc(NC(=O)C3CC3)ncc12]

### Step 1: 2-(3-bromo-1-pyrazol-1-yl)acetonitrile

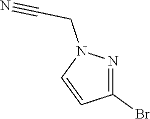

A mixture of 3-bromo-1H-pyrazole (150 mg; 1.02 mmol; 1.00 eq.), 2-bromoacetonitrile (159.1 mg; 1.327 mmol; 1.5 eq.) and Cs₂CO₃ (665 mg; 2.04 mmol; 2.00 eq.) in DMF (5 mL) was stirred for 6 h at room temperature. The reaction was monitored by LCMS. The reaction was diluted with EtOAc (80 mL) and washed with brine (2×10 mL), dried over anhydrous Na₂SO₄. After filtration, the filtrate was concentrated under reduced pressure. The residue was purified by flash chromatography on pre-packed C18 column using 20-50% MeCN in water (10 mmol/L NH₄HCO₃) to provide 2-(3-bromo-1H-pyrazol-1-yl)acetonitrile as a brown solid (140 mg, 74%). LCMS (ESI) m/z 186.0, [M+H]⁺.

### Step 2: N-(5-(1-(cyanomethyl)-1H-pyrazol-3-yl)-8-(methylamino)-2,7-naphthyridin-3-yl)cyclopropanecarboxamide

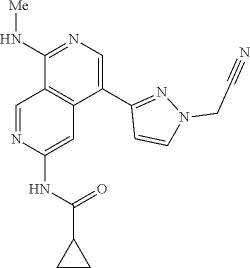 [SMILES: CNc1ncc(-c2ccn(CC#N)n2)c2cc(NC(=O)C3CC3)ncc12]

A mixture of 2-(3-bromo-1H-pyrazol-1-yl)acetonitrile (100 mg; 0.540 mmol: 2.00 eq.), N-(8-(methylamino)-5-(4,4,5,5-tetramethyl-1,3,2-dioxaborolan-2-yl)-2,7-naphthyridin-3-yl)cyclopropanecarboxamide (Example 81, step 1) (100 mg, 0.272 mmol, 1.00 equiv), Pd(DtBPF)Cl₂ (17.7 mg; 0.027 mmol; 0.10 eq.) and K₃PO₄(115.3 mg; 0.544 mmol; 2.00 eq.) in 1,4-dioxane/water (10:1, 2.2 mL) was stirred for 2 h at 90° C. under nitrogen atmosphere. The reaction was monitored by LCMS. The residue was purified by flash chromatography on silica gel column using MeOH/CH₂Cl₂ (2-10%) as eluent to afford. N-(5-(1-(cyanomethyl)-1H-pyrazol-3-yl)-8-(methylamino)-2,7-naphthyridin-3-yl)cyclopropanecarboxamide (10 mg, crude). The crude product was purified by flash chromatography on pre-packed. C18 column using 20-50% MeCN in water (10 mmol/L NH₄HCO₃) to provide N-(5-(1-(cyanomethyl)-1H-pyrazol-3-yl)-8-(methylamino)-2,7-naphthyridin-3-yl)cyclopropanecarboxamide as a white solid (2.7 mg, 3%). LCMS (ESI) m/z 348.1, [M+H]⁺. ¹H NMR (400 MHz, DMSO-d₆) δ 10.93 (s, 1H), 9.38 (s, 1H), 8.82 (s, 1H), 8.22 (s, 8.10-7.89 (m, 2H), 6.62 (s, 1H), 5.54 (s, 2H), 3.02 (d, J=4.4 Hz, 3H), 2.15-1.98 (m, 1H), 0.97-0.70 (m, 4H).

### Example 88: Synthesis of N-(8-(methylamino)-5-(2,2,2-trifluoroethyl)-2,7-naphthyridin-3-yl)cyclopropanecarboxamide

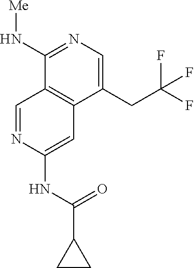 [SMILES: CNc1ncc(CC(F)(F)F)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (Example 81, step 1) (150 mg; 0.407 mmol; 1.00 eq.), 1,1,1-trifluoro-2-iodoethan (256.7 mg; 1.2.2 mmol; 3.00 eq.), XPhos Pd G₃ (34.5 mg; 0.040 mmol; 0.10 eq.), XPhos (19.4 mg; 0.040 mmol; 0.10 eq.) and K₃PO₄ (259.2 mg; 1.22 mmol; 3.00 eq.) in 1,4-dioxane/water (10:1, 2.2 mL, deoxygenated prior to use) was stirred for 3 h at 90° C. under nitrogen atmosphere. The reaction was monitored by LCMS. The resulting mixture was concentrated under reduced pressure. The residue was purified by flash chromatography on silica gel column using 2-10% of MeOH in CH₂Cl₂ as eluent to afford N-(8-(methylamino)-5-(2,2,2-de (20 mg, crude). The crude product was purified by flash chromatography on pre-packed C18 column using 20-50% MeCN in water (10 mmol/L NH₄HCO₃) to provide N-(8-(methylamino)-5-(2,2,2-trifluoroethyl)-2,7-naphthyridin-3-yl)cyclopropanecarboxamide as a white solid. (10.1 mg, 4%). LCMS (ESI) m/z 325.1, [M+H]⁺. ¹H NMR (400 Hz, DMSO-d₆) δ 11.02 (s, 1H), 9.34 (s, 1H), 8.38 (s, 1H), 8.10-7.98 (m, 2H), 3.75-3.60 (m, 2H), 2.97 (d, J=4.4 Hz, 3H), 2.12-2.01 (m, 1H), 0.95-0.80 (m, 4H).

### Example 89: Synthesis of N-(5-(3-ethoxypyridin-2-yl-8-(methylamino)-2,7-naphthyridin-3-yl) acetamide

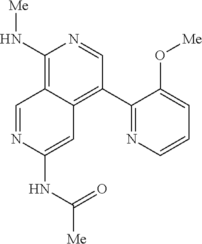 [SMILES: CNc1ncc(-c2ncccc2OC)c2cc(NC(C)=O)ncc12]

### Step 1: N-(8-hydroxy-2,7-naphthyridin-3-yl)acetamide

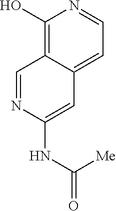 [SMILES: CC(=O)Nc1cc2ccnc(O)c2cn1]

A mixture of Pd₂(dba)₃ (250 mg; 0.273 mmol; 0.10 eq.) and XantPhos (325 mg; 0.562 mmol; 0.20 eq.), 6-chloro-2,7-naphthyridin-1-ol (500 mg; 2.77 mmol; 1.00 eq.), acetamide (650 mg; 11.0 mmol; 4.00 eq.) and Cs₂CO₃ (10.8 g; 5.53 mmol; 2.00 eq.) in dioxane (12 mL) was stirred at 110° C. for 2 h under nitrogen atmosphere. The desired product was observed via LCMS. The reaction was concentrated in vacuo, the residue was purified by flash chromatography on silica gel column using 30-100% of EtOAc in petroleum ether and 2-20% MeOH in CH₂Cl₂ as eluent to give crude product. The crude product was diluted with plenty CH₂Cl₂ (20 mL), then filtered, the filter cake was washed with plenty CH₂Cl₂ and dried over to give N-(8-hydroxy-2,7-naphthyridin-3-yl)acetamide as yellow solid (560 mg, 99%). LCMS (ESI) m/z 204.1 [M+H]⁺.

### Step 2: N-(8-chloro-2,7-naphthyridin-3-yl)acetamide

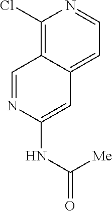 [SMILES: CC(=O)Nc1cc2ccnc(Cl)c2cn1]

N-(8-hydroxy-2,7-naphthyridin-3-yl)acetamide (250 mg; 1.23 mmol; 1.00 eq.) was dissolved in POCl₃ (6 mL) under nitrogen atmosphere. The reaction was stirred at 100° C. for 30 min. The desired product was monitored via LCMS. The reaction was cooled to rt and concentrated in vacuo, the resulting mixture was diluted with 100 mL CH₂Cl₂ and neutralized to pH 7 with a saturated NaHCO₃ solution. The organic phase was concentrated in vacuo. The residue was purified by flash chromatography on silica gel column using 10-30% of EtOAc in CH₂Cl₂ to give N-(8-chloro-2,7-naphthyridin-3-yl)acetamide as a light yellow solid (80 mg, 29%). LCMS (ER) m/z 222.0, [M+H]⁺.

### Step 3: N-(8-(methylamino)-2,7-naphthyridin-3-yl)acetamide

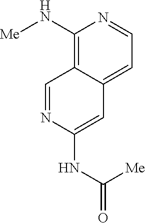 [SMILES: CNc1nccc2cc(NC(C)=O)ncc12]

N-(8-chloro-2,7-naphthyridin-3-yl)acetamide (80 mg; 0.361 mmol, 1.00 eq.) was added to a solution of methylamine in THF (2 M, 4 mL). The reaction was stirred at 60° C. for overnight. The progress of the reaction was monitored via LCMS. To this mixture was added an additional of methylamine solution in THF (2 M, 4 mL). The reaction was stirred at 60° C. for overnight. Upon completion, the resulting mixture was concentrated under vacuum. The residue was purified by flash chromatography on silica gel column using 2-14% of MeOH in CH₂Cl₂ to give N-(8-(methylamino)-2,7-naphthyridin-3-yl)acetamide as off-white solid (75 mg, 96%). LCMS (ESI) m/z 217.1, [M+H]⁺.

### Step 4: N-(5-bromo-8-(methylamino)-2,7-naphthyridin-3-yl)acetamide

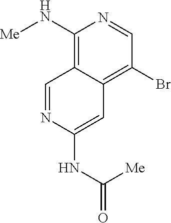 [SMILES: CNc1ncc(Br)c2cc(NC(C)=O)ncc12]

To a stirring mixture of N-(8-(methylamino)-2,7-naphthyridin-3-yl)acetamide (141 mg; 0.652 mmol; 1.00 eq.) in DMF (4 mL) at 0° C. was added NBS (116 mg; 0.652 mmol; 1.00 eq.) under nitrogen atmosphere. Then the reaction was stirred at room temperature for 1 h. Upon completion, the mixture was diluted with EtOAc (80 mL) and washed with brine (20 mL×5). The organic layer was dried, concentrated under vacuum and purified by flash chromatography on silica gel column using 2-6% MeOH in CH₂Cl₂ to give N-(5-bromo-8-(methylamino)-2,7-naphthyridin-3-yl)acetamide as off-white solid (166 mg, 86%). LCMS (ESI) m/z 295.0, [M+H]⁺.

### Step 5: N-(5-(3-methoxypyridin-2-yl)-8-(methylamino)-2,7-naphthyridin-3-yl)acetamide

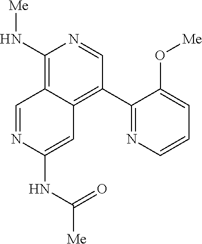 [SMILES: CNc1ncc(-c2ncccc2OC)c2cc(NC(C)=O)ncc12]

A mixture of LiCl (16.7 mg; 0.394 mmol; 2.50 eq.), CuI (6.1 mg; 0.032 mmol; 0.20 eq.) and Pd(PPh₃)₄. (37 mg; 0.032 mmol; 0.2.0 eq.) in dioxane (2.5 mL, deoxygenated prior to use), was added to N-(5-bromo-8-(methylamino)-2,7-naphthyridin-3-yl)acetamide (47 mg; 0.159 mmol; 1.00 eq.) and 3-methoxy-2-(tributylstannyl)pyridine (319 mg; 0.341 mmol; 5.00 eq.). The reaction mixture was stirred at 110° C. for 1 h under nitrogen atmosphere. The desired product was observed via LCMS. The reaction mixture was cooled to rt and concentered. The resulting residue was purified by flash chromatography on silica gel column using MeOH/CH₂Cl₂ (2-20%) to afford the crude desired product. The crude product was further purified by flash chromatography on pre-packed C18 column using 10-30% of MeCN in water (10 mmol/L NH₄HCO₃) to afford N-(5-(3-methoxypyridin-2-yl)-8-(methylamino)-2,7-naphthyridin-3-yl)acetamide as white solid (31.5 mg, 61%). LCMS (ESI) m/z 324.1, [M+H]⁺. ¹H NMR (400 MHz, DMSO-d₆) δ 10.54 (s, 1H), 9.34 (s, 1H), 8.28-8.24 (m, 1H), 8.01 (s, 1H), 8.00-7.97 (m, 2H), 7.60-7.56 (m, 1H), 7.44-7.39 (m, 1H), 3.73 (s, 3H), 3.02 (d, J=4.4 Hz, 3H), 2.05 (s, 3H).

### Example 90: Synthesis of N-(8-(methylamino)-5-phenyl-2,7-naphthyridin-3-yl)acetamide

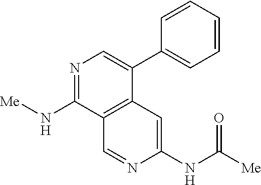 [SMILES: CNc1ncc(-c2ccccc2)c2cc(NC(C)=O)ncc12]

To a stirring mixture of N-(5-bromo-8-(methylamino)-2,7-naphthyridin-3-yl)acetamide (Example 89, step 4) (40 mg; 0.136 mmol; 1.00 eq.) in dioxane/water (10:1, 2.2 mL), was added to phenylboronic acid (33 mg, 0.272 mmol; 2.00 eq.), Pd(DtBPF)Cl₂ (8.8 mg; 0.014 mmol; 0.10 eq.) and K₃PO₄ (57.5 mg, 0.272 mmol; 0.20 eq.) under nitrogen atmosphere. The reaction was stirred at 90° C. for 2 h. The reaction mixture was cooled to rt. The resulting mixture was concentrated under reduced pressure. The residue was purified by flash chromatography on silica gel column using MeOH in CH₂Cl₂ (1-8%) as eluent to provide the crude product. The crude product was further purified by flash chromatography on pre-packed C18 column using 10-43% of MeCN in water (10 mmol/L NH₄HCO₃) as eluent to provide N-(8-(methylamino)-5-phenyl-2,7-naphthyridin-3-yl)acetamide as a off-white solid (12.0 mg, 30%). LCMS (ESI) m/z 293.1, [M+H]⁺. ¹H NMR (400 MHz, DMSO-d₆) δ 10.63 (s, 1H), 9.37 (s, 1H), 8.36 (s, 1H), 7.99-7.96 (m, 1H), 7.95 (s, 1H), 7.52-7.37 (m, 5H), 3.01 (d, J=4.4 Hz, 3H), 2.07 (s, 3H).

### Example 91: Synthesis of 4-(3-methoxypyridin-2-yl)-N¹-methyl-N⁶-(pyridin-2-yl)-2,7-naphthyridine-1,6-diamine

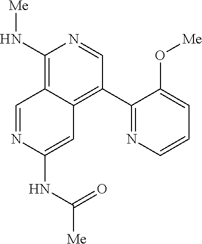 [SMILES: CNc1ncc(-c2ncccc2OC)c2cc(NC(C)=O)ncc12]

### Step 1: 6-chloro-4-iodo-2,7-naphthyridin-1-ol

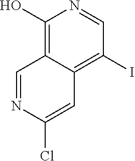 [SMILES: Oc1ncc(I)c2cc(Cl)ncc12]

A mixture of 6-chloro-2,7-naphthyridin-1-ol. (1 g; 5.53 mmol; 1.00 eq.) and 1-iodopyrrolidine-2,5-di one (1.87 g; 8.30 mmol; 1.50 eq.) in DMF (10 mL) was stirred for 5 h at room temperature under nitrogen atmosphere. The resulting mixture was quenched by adding water dropwise (50 mL) and stirred for 30 min at room temperature and there were a lot of precipitate formed. The mixture was filtered, and the solid was washed with water (10 mL). The solids were dried under reduced pressure to provide 6-chloro-4-iodo-2,7-naphthyridin-1-ol as a yellow solid (1.05 g, 61%). LCMS (ESI) m/z 306.9, [M+H]⁺.

### Step 2: 6-chloro-4-(3-methoxypyridin-2-yl)-2,7-naphthyridin-1(2H)-one

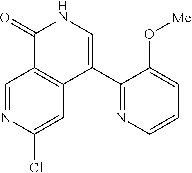 [SMILES: COc1cccnc1-c1c[nH]c(=O)c2cnc(Cl)cc12]

A mixture of 6-chloro-4-iodo-2,7-naphthyridin-1-ol (600 mg; 1.96 mmol; 1.00 eq.), 3-methoxy-2-(tributylstannyl)pyridine (2.34 g; 5.874 mmol; 3.00 eq.), Pd(PPh₃)₂Cl₂ (137 mg; 0.196 mmol; 0.10 eq.) and CuI (74.5 mg; 0.392 mmol; 0.20 eq.) in dioxane (5 mL) was stirred for 18 h at 60° C. under nitrogen atmosphere. The reaction was monitored by LCMS. The reaction mixture was cooled to rt and concentered. The resulting residue was purified flash chromatography on silica gel column using MeOH/CH₂Cl₂(2-10%) as eluent to provide 6-chloro-4-(3-methoxypyridin-2-yl)-2,7-naphthyridin-1(2H)-one as a yellow solid (240 mg, 42%). LCMS (ESI) m/z 288.0, [M+H]⁺.

### Step 3: 4-(3-ethoxypyridin-2-yl)-6-(pyridin-2-ylamino)-2,7-naphthyridin-1(2H)-one

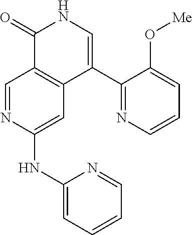 [SMILES: COc1cccnc1-c1c[nH]c(=O)c2cnc(Nc3ccccn3)cc12]

A mixture of 6-chloro-4-(3-methoxypyridin-2-yl)-2,7-naphthyridin-1(2H)-one (330 mg; 1.14 mmol; 1.00 eq.), pyridin-2-amine (216 mg; 2.29 mmol; 2.00 eq.), Pd₂(dba)₃ (105 mg; 0.115 mmol; 0.10 eq.), XantPhos (132 mg; 0.229 mmol; 0.20 eq.) and Cs₂CO₃ (934 mg; 2.87 mmol; 2.50 eq.) in dioxane (5 mL) was stirred for overnight at 110° C. under nitrogen atmosphere. The reaction was monitored by LCMS. The reaction mixture was cooled to rt, concentrated under reduced pressure and purified flash chromatography on silica gel column using MeOH/CH₂Cl₂ (5-20%) as eluent to provide 4-(3-methoxypyridin-2-yl)-6-(pyridin-2-ylamino)-2,7-naphthyridin-1(2H)-one as a white solid (350 mg, 88%). LCMS (ESI) m/z 346.1, [M+H]⁺.

### Step 4: 8-chloro-5-(3-methoxypyridin-2-yl)-N-(pyridin-2-yl)-2,7-naphthyridin-3-amine

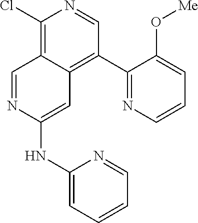 [SMILES: COc1cccnc1-c1cnc(Cl)c2cnc(Nc3ccccn3)cc12]

4-(3-methoxypyridin-2-yl)-6-(pyridin-2-ylamino)-2,7-naphthyridin-1(2H)-one (200 mg; 0.579 mmol; 1.00 eq.) and POCl₃ (5 mL) were mixed in a reaction flask. The resulting mixture was stirred for 1 h at 100° C. The reaction was monitored by LCMS. The resulting mixture was concentrated under reduced pressure to afford 8-chloro-5-(3-methoxypyridin-2-yl)-N-(pyridin-2-yl)-2,7-naphthyridin-3-amine as a brown solid (200 mg; 95%). LCMS (ESI) m/z 364.1, [M+H]⁺.

### Step 5: 4-(3-methoxypyridin-2-yl)-N¹-methyl-N⁶-(pyridin-2-yl)-2,7-naphthyridine-1,6-diamine

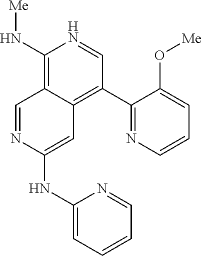

A mixture of 8-chloro-5-(3-methoxypyridin-2-yl)-N-(pyridin-2-yl)-2,7-naphthyridin-3-amine (200 mg; 0.551 mmol; 1.00 eq.), methanamine hydrochloride (184 mg; 2.73 mmol; 5.00 eq.) and DIPEA (706 mg; 5.47 mmol; 2.00 eq.) in NMP (5 mL) was stirred for 16 h at 100° C. The reaction was monitored by LCMS. The mixture was cooled down and concentrated. The resulting residue was purified by flash chromatography on pre-packed C18 column using 20-50% of MeCN in water (10 mmol/L NH₄HCO₃) as eluent to provide 4-(3-methoxypyridin-2-yl)-N¹-methyl-N⁶-(pyridin-2-yl)-2,7-naphthyridine-1,6-diamine as a yellow solid (60 mg, crude). The crude product was purified by reverse phase preparative HPLC (Prep-C18, XBridge Prep OBD C18 Column, 30×150 mm, waters; gradient elution of 35-45% MeCN in water over a 10 min period, where both water and MeCN contain 10 mmol/L NH₄HCO₃, flow rate: 60 mL/min, detector UV wavelength: 220 nm) to provide 4-(3-methoxypyridin-2-yl)-N¹-methyl-10-(pyridin-2-yl)-2,7-naphthyridine-1,6-diamine as a yellow solid (35.2 mg, 17%). LCMS (ESI) m/z 359.2, [M+H]⁺. ¹H NMR (400 MHz; DMSO-d₆) δ 9.78 (s, 1H), 9.29 (s, 1H), 8.28 (d, J=4.4 Hz, 1H), 8.06-8.03 (m, 1H), 8.00 (s, 1H), 7.96 (s, 1H), 7.92-7.87 (m, 1H), 7.68-7.59 (m, 2H), 7.48-7.34 (m, 2H), 6.86-6.78 (m, 1H), 3.74 (s, 3H), 3.02 (d, J=4.4 Hz, 3H).

### Example 92: Synthesis of N¹-methyl-4-phenyl-N⁶-(pyridin-yl)-2,7-naphthyridine-1,6-diamine

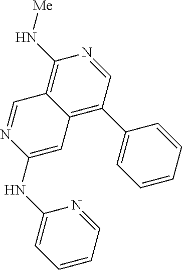 [SMILES: CNc1ncc(-c2ccccc2)c2cc(Nc3ccccn3)ncc12]

### Step 1: 6-chloro-4-phenyl-2,7-naphthyridin-1(2H)-one

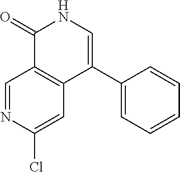 [SMILES: O=c1[nH]cc(-c2ccccc2)c2cc(Cl)ncc12]

A mixture of 6-chloro-4-iodo-2,7-naphthyridin-1-ol (Example 91, step 1) (400 mg; 1.305 mmol; 1.00 eq.), phenylboronic acid (477 mg; 3.91 mmol; 3.00 eq.), Pd(PPh₃)₄ (151 mg; 0.131 mmol, 0.10 eq.) and Na₂CO₃ (748 mg; 7.06 mmol; 5.41 eq.) in a mixture solvent of DME/water (5:1, 12 mL) was stirred for 1 h at 100° C. under nitrogen atmosphere. The reaction mixture was cooled to rt and the crude product mixture was concentrated under reduced pressure. The residue was purified flash chromatography on silica gel column using MeOH/CH₂Cl₂ (2-10%) eluent to provide 6-chloro-4-phenyl-2,7-naphthyridin-1(2H)-one as a yellow solid (228 mg, 68%). LCMS (ESI) m/z 257.0, [M+H]⁺.

### Step 2: 4-phenyl-6-(pyridin-2-ylamino)-2,7-naphthyridin-1(2H)-one

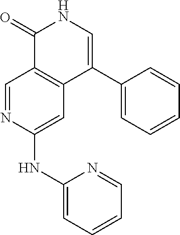 [SMILES: O=c1[nH]cc(-c2ccccc2)c2cc(Nc3ccccn3)ncc12]

A mixture of 6-chloro-4-phenyl-2,7-naphthyridin-1(2H)-one (200 mg; 0.779 mmol; 1.00 eq.), pyridin-2-amine (220 mg; 2.33 mmol; 3.00 eq.), Pd₂(dba)₃ (71.4 mg; 0.078 mmol; 0.10 eq.), XantPhos (90.2 mg; 0.156 mmol; 0.20 eq.) and Cs₂CO₃ (507.8 mg; 1.55 mmol; 2.00 eq.) in 1,4-dioxane (10 mL) was stirred for 2 h at 100° C. under nitrogen atmosphere. The desired product was observed via LCMS. The reaction mixture was concentrated under reduced pressure. The residue was purified flash chromatography on silica gel column using 2-10% of MeOH in CH₂Cl₂ as eluent to provide 4-phenyl-6-(pyridin-2-ylamino)-2,7-naphthyridin-1(2H)-one (98 mg, 40%). LCMS (ESI) m/z 315.1, [M+H]⁺.

### Step 3: 8-chloro-5-phenyl-N-(pyridin-2-yl)-2,7-naphthyridin-3-amine

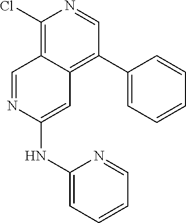 [SMILES: Clc1ncc(-c2ccccc2)c2cc(Nc3ccccn3)ncc12]

4-phenyl-6-(pyridin-2-ylamino)-2,7-naphthyridin-1(2H)-one (98 mg; 0.312 mmol; 1.00 eq.) was mixed with in POCl₃ (4 mL) and the mixture was stirred for 1 h at 100° C. under nitrogen atmosphere. Upon completion, the mixture was allowed to cool down to room temperature. The resulting mixture was concentrated under reduced pressure. The residue was dissolved in CH₂Cl₂ (50 mL). The mixture was basified to pH 7 with a sat. NaHCO₃ solution. The resulting mixture was extracted with CH₂Cl₂ (2×40 mL). The combined organic layers were washed with a sat. NaCl solution (1×5 mL), dried over anhydrous Na₂SO₄. After filtration, the filtrate was concentrated under reduced pressure. The residue was purified flash chromatography on silica gel column using 20-80% of EtOAc in petroleum ether as eluent to provide 8-chloro-5-phenyl-N-(pyridin-2-yl)-2,7-naphthyridin-3-amine as a light-yellow solid (43.2 mg, 41%). LCMS (ESI) 333.1, [M+H]⁺.

### Step 4: N¹-methyl-4-phenyl-N⁶-(pyridin-2-yl)-2,7-napthyridine-1,6-diamine

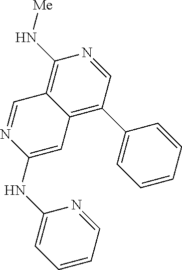 [SMILES: CNc1ncc(-c2ccccc2)c2cc(Nc3ccccn3)ncc12]

A mixture of 8-chloro-5-phenyl-N-(pyridin-2-yl)-2,7-naphthyridin-3-amine (43.2 mg; 0.130 mmol; 1.00 eq.), methanamine hydrochloride (18 mg; 0.267 mmol; 2.05 eq.) and DIPEA (83.9 mg 0.649 mmol; 5.00 eq.) in NMP (2 mL) was stirred for 12 h at 100° C. under nitrogen atmosphere. The reaction was cooled to rt. The resulting mixture was purified by flash chromatography on pre-packed C18 column using 20-50% of MeCN in water (10 mmol/L NH₄HCO₃) as eluent to provide N¹-methyl-4-phenyl-N⁶-(pyridin-2-yl)-2,7-naphthyridine-1,6-diamine as a white solid (20.0 mg, 47%). LCMS (ESI) m/z 328.1, [M+H]⁺. NMR (400 MHz, DMSO-d₆) δ 9.90 (s, 1H), 9.32 (s, 1H), 8.42 (s, 1H), 8.10-8.06 (m, 1H), 7.97-7.91 (m, 1H), 7.88 (s, 1H), 7.67-7.61 (m, 1H), 7.55-7.49 (m, 4H), 7.44-7.35 (m, 2H), 6.87-682 (m, 1H), 3.01 (d, J=4.4 Hz, 3H).

### Example 93: Synthesis of N⁶-(cyclopropylmethyl)-N¹-methyl-4-phenyl-2,7-naphthyridine-1,6-diamine

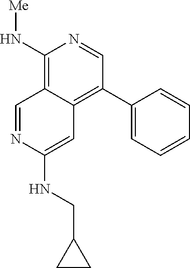 [SMILES: CNc1ncc(-c2ccccc2)c2cc(NCC3CC3)ncc12]

### Step 1: 6-((cyclopropylmethyl)amino)-4-phenyl-2,7-naphthyridin-1(2H)-one

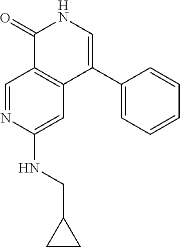 [SMILES: O=c1[nH]cc(-c2ccccc2)c2cc(NCC3CC3)ncc12]

A mixture of 6-chloro-4-phenyl-2,7-naphthyridin-1(2H)-one (Example 92, step 1) (261 mg; 1.02 mmol; 1.00 eq.), cyclopropylmethanamine (725 mg; 10.1 mmol; 10.0 eq.) and DIPEA (329 mg; 2.55 mmol; 2.50 eq.) in NMP (5 mL) was stirred for 12 h at 100° C. under nitrogen atmosphere. The reaction was cooled to rt. The resulting mixture was purified by flash chromatography on pre-packed C18 column using 20-50% of MeCN in water (10 mmol/L NH₄HCO₃) as eluent to provide 6-((cyclopropylmethylamino)-4-phenyl-2,7-naphthyridin-1(2H)-one as a light-yellow solid (145 mg, 48%). LCMS (ESI) m/z 292.1 [M+H]⁺.

### Step 2: 8-chloro-N-(cyclopropylmethyl)-5-phenyl-2,7-naphthyridin-3-amine

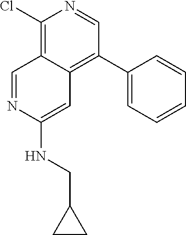 [SMILES: Clc1ncc(-c2ccccc2)c2cc(NCC3CC3)ncc12]

6-((cyclopropylmethylamino)-4-phenyl-2,7-naphthyridin-1(2H)-one (145 mg; 0.498 mmol, 1.00 eq.) was dissolved in POCl₃ (4 mL) and stirred for 1 at 100° C. under nitrogen atmosphere. The mixture was allowed to cool down to room temperature. The resulting mixture was concentrated under reduced pressure. The residue was dissolved in CH₂Cl₂ (55 mL). The mixture was basified to pH 7 with a sat. NaHCO₃ solution. The resulting mixture was extracted with CH₂Cl₂ (2×40 mL). The combined organic layers were washed with a sat. NaCl solution (1×5 mL), dried over anhydrous-Na₂SO₄. After filtration, the filtrate was concentrated under reduced pressure. The residue was purified by flash chromatography on silica gel column using EtOAc/petroleum ether (10-80%) as eluent to provide 8-chloro-N-(cyclopropylmethyl)-5-phenyl-2,7-naphthyridin-3-amine as a yellow solid (84.7 mg, 54%). LCMS (ESI) m/z 310.1, [M+H]⁺.

### Step 3: N⁶⁻(cyclopropylmethyl)-N¹-methyl-4-phenyl-2,7-naphthyridine-1,6-diamine

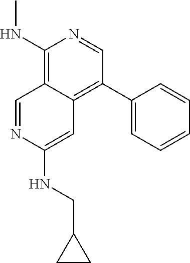 [SMILES: CNc1ncc(-c2ccccc2)c2cc(NCC3CC3)ncc12]

A mixture of 8-chloro-N-(cyclopropylmethyl)-5-phenyl-2,7-naphthyridin-3-amine (74.7 mg; 0.241 mmol; 1.00 eq.), methyl amine hydrochloride salt (48.6 mg; 0.72 mmol; 2.99 eq.) and DIPEA (155 mg; 1.20 mmol; 4.99 eq.) in NMP (3 mL) was stirred for 12 h at 120° C. under nitrogen atmosphere. The reaction mixture was cooled to rt and the resulting mixture was purified by flash chromatography on pre-packed C18 column using 20-50% of MeCN in water (10 mmol/L NH₄HCO₃) as eluent to provide N⁶-(cyclopropylmethyl)-N⁶-methyl-4-phenyl-2,7-naphthyridine-1,6-diamine as a white solid (33.5 mg, 45%). LCMS (ESI) m/z 305.2, [M+H]⁺. ¹H-NMR (400 MHz, DMSO-d₆) δ 9.10 (s, 1H), 7.66 (s, 1H), 7.65-7.62 (m, 1H), 7.49-7.44 (m, 2H), 7.41-7.34 (m, 3H), 6.91-6.86 (m, 1H), 6.41 (s, 1H), 3.13-3.08 (m, 2H), 2.96 (d, J=4.4 Hz, 3H), 1.04-0.93 (m, 1H), 0.44-0.38 (m, 2H), 0.19-0.14 (m, 2H).

### Example 94: Synthesis of N¹-methyl-4-phenyl-N⁶-(2,2,2-trifluoroethyl)-2,7-naphthyridine-1,6-diamine

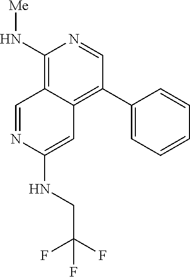 [SMILES: CNc1ncc(-c2ccccc2)c2cc(NCC(F)(F)F)ncc12]

### Step 1: 4-phenyl-6-((2,2,2-trifluormethyl)amino)-2,7-naphthyridin-1(2H)-one

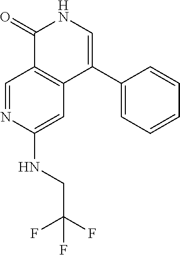 [SMILES: O=c1[nH]cc(-c2ccccc2)c2cc(NCC(F)(F)F)ncc12]

A mixture of 6-chloro-4-phenyl-2,7-naphthyridin-1(2H)-one (Example 92, step 1) (100 mg; 0.390 mmol; 1.00 eq.), 2,2,2-trifluoroethan-1-amine (77.2 mg; 0.779 mmol; 2.00 eq.), Pd-PEPPSI-IHeptCl (37.9 mg; 0.039 mmol; 0.10 eq.) and t-BuOK (87.4 mg; 0.779 mmol; 2.00 eq.) in 1,4-dioxane (10 mL) was stirred at 90° C. for 16 h under nitrogen atmosphere. The reaction was cooled to rt and the mixture was concentrated under reduced pressure. The residue was purified by flash chromatography on silica gel column using MeOH/CH₂Cl₂ (2-10%) as eluent to provide 4-phenyl-6-((2,2,2-trifluoroethyl)amino)-2,7-naphthyridin-1(2H)-one as an off-white solid (80.6 mg, 64%). LCMS (ESI) m/z 320.1, [M+H]⁺.

### Step 2: 8-chloro-5-phenyl-N-(2,2,2-trifluormethyl)-2,7-naphthyridin-3-amine

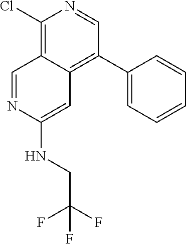 [SMILES: FC(F)(F)CNc1cc2c(-c3ccccc3)cnc(Cl)c2cn1]

4-phenyl-6-((2,2,2-trifluoroethyl)amino)-2,7-naphthyridin-1(2H)-one (80.6 mg; 0952 mmol; 1.00 eq.) was dissolved in POCl₃ (3 mL). The reaction mixture was stirred for 1 h at 100° C. under nitrogen atmosphere. The mixture was allowed to cool down to room temperature. The resulting mixture was concentrated under reduced pressure. The residue was dissolved in CH₂Cl₂ (50 mL). The mixture was basified to pH 7 with a sat. NaHCO₃ solution. The resulting mixture was extracted with CH₂Cl₂(2×40 mL). The combined organic layers were washed with a sat, —NaCl solution (1×5 mL), dried over anhydrous Na₂SO₄. After filtration, the filtrate was concentrated under reduced pressure. The residue was purified by flash chromatography on silica gel column using 20-80% of EtOAc in petroleum ether as eluent to provide 8-chloro-5-phenyl-N-(2,2,2-trifluoroethyl)-2,7-naphthyridin-3-amine as a yellow solid (76.6 mg, 89%), LCMS (ESI) m/z 338.1, [M+H]⁺.

### Step 3: N¹-methyl-4-phenyl-N⁶-(2,2,2-trifluoroethyl)-2,7-naphthyridine-1,6-diamine

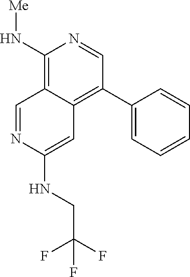 [SMILES: CNc1ncc(-c2ccccc2)c2cc(NCC(F)(F)F)ncc12]

A mixture of 8-chloro-5-phenyl-N-(2,2,2-trifluoroethyl)-2,7-naphthyridin-3-amine (66.6 mg; 0.197 mmol; 1.00 eq.), methanamine hydrochloride (39.9 mg; 0.591 mmol; 3.00 eq.) and DIPEA (127 mg; 0.984 mmol; 4999 eq.) in NMP (3 mL) was stirred for 12 h at 120° C. under nitrogen atmosphere. The reaction was cooled to rt. The resulting mixture was purified by flash chromatography on pre-packed C18 column using 20-50% of MeCN in water (10 mmol/L NH₄HCO₃) as eluent to provide N¹-methyl-4-phenyl-N⁶)-(2,2,2-trifluoroethyl)-2,7-naphthyridine-1,6-diamine as a white solid (41.3 mg, 63%). LCMS (ESI) m/z 333.1, [M+H]⁺. ¹H NMR (400 MHz, DMSO-d₆) δ 9.17 (s, 1H), 7.74 (s, 1H), 7.73-7.70 (m, 1H), 7.51-7.45 (m, 2H), 7.41-7.36 (m, 4H), 6.64 (s, 1H), 4.25-4.14 (m, 2H), 2.98 (d, J=4.4 Hz, 3H).

### Example 95: Synthesis of N-(8-chloro-5-phenyl-2,7-naphthyridin-3-yl)cyclopropanecarboxamide

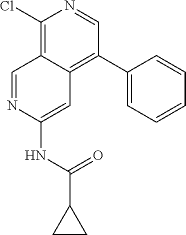 [SMILES: O=C(Nc1cc2c(-c3ccccc3)cnc(Cl)c2cn1)C1CC1]

### Step 1: N-(8-hydroxy-5-phenyl-2,7-naphthyridin-3-yl)cyclopropanecarboxamide

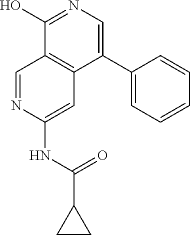 [SMILES: O=C(Nc1cc2c(-c3ccccc3)cnc(O)c2cn1)C1CC1]

To a stirring mixture of 6-chloro-4-phenyl-2,7-naphthyridin-1(2H)-one (Example 92, step 1) (1.1 g; 4.29 mmol; 1.00 eq.) in dioxane (30 mL) were added cyclopropanecarboxamide (1.46 g; 17.1 mmol; 4.00 eq.), Pd₂(dba)₃ (394.4 mg; 0.429 mmol; 0.10 eq.), XantPhos (495 mg; 0.859 mmol; 0.20 eq.) and Cs₂CO₃ (2.8 g; 8.58 mmol; 2.00 eq.) under nitrogen atmosphere. The reaction was stirred under nitrogen at 130 for overnight. Upon completion, the reaction was concentrated under reduced pressure and purified by flash chromatography on silica gel column using MeOH/CH₂Cl₂(3˜15%) to give N-(8-hydroxy-5-phenyl-2,7-naphthyridin-3-yl)cyclopropanecarboxamide as a light yellow solid (560 mg, crude). The crude product was diluted with water (20 mL), then filtered, the filter cake was washed with water and dried over to give N-(8-hydroxy-5-phenyl-2,7-naphthyridin-3-yl)cyclopropanecarboxamide as a yellow solid (180 mg, 13%). LCMS (ESI) m/z 306.1, [M+H]⁺.

### Step 2: N-(8-chloro-5-phenyl-2,7-naphthyridin-3-yl)cyclopropanecarboxamide

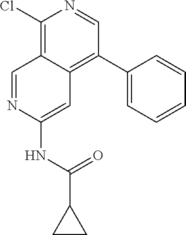 [SMILES: O=C(Nc1cc2c(-c3ccccc3)cnc(Cl)c2cn1)C1CC1]

N-(8-hydroxy-5-phenyl-2,7-naphthyridin-3-yl)cyclopropanecarboxamide (116 mg; 0.380 mmol; 1.00 eq.) was dissolved in phosphorus oxychloride (3 mL). The reaction was stirred under nitrogen at 100° C. for 1 h. Upon completion, the reaction was concentrated under reduced pressure. The resulting mixture was diluted with CH₂Cl₂ (50 mL). The resulting mixture was washed with a saturated NaHCO₃ solution. The solution was dried with Na₂SO₄, filtered and the solvent was removed under reduced pressure. The residue was purified by flash chromatography on silica gel column using 20-50% of EtOAc in petroleum ether to afford the crude product. The crude was further purified by reverse phase preparative HPLC (Prep-C18, 5 μM OBD column, 19×250 mm, waters; gradient elution of 46-56% MeCN in water over a 8 min period, where both water and MeCN contain 10 mmol/L NH₄HCO₃, flow rate: 60 mL/min, detector UV wavelength: 254 nm) to afford N-(8-chloro-5-phenyl-2,7-naphthyridin-3-yl)cyclopropanecarboxamide as a white solid (1.9 mg, 1%). LCMS (ESI) m/z 324.1, [M+H]⁺. ¹H NMR (400 DMSO-d₆) δ 11.35 (s, 1H), 9.52 (s, 1H), 8.54 (s, 1H), 8.33 (s, 1H), 7.62-7.51 (m, 5H), 2.10-2.03 (m, 1H), 0.85-0.78 (m, 4H).

### Example 96: Synthesis of N-(8-ethyl-5-phenyl-2,7-naphthyridin-3-yl)cyclopropanecarboxamide

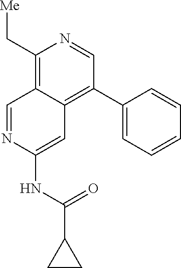 [SMILES: CCc1ncc(-c2ccccc2)c2cc(NC(=O)C3CC3)ncc12]

### Step 1: N-(5-phenyl-8-vinyl-2,7-naphthyridin-3-yl)cyclopropanecarboxamide

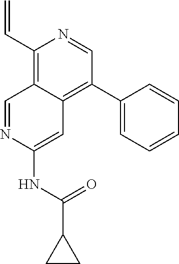 [SMILES: C=Cc1ncc(-c2ccccc2)c2cc(NC(=O)C3CC3)ncc12]

To a stirring mixture of N-(8-chloro-5-phenyl-2,7-naphthyridin-3-yl)cyclopropanecarboxamide (Example 95, step 2) (80 mg; 0.247 mmol; 1.00 eq.) dioxane/water (5:1, 6 mL), were added 4,4,5,5-tetramethyl-2-vinyl-1,3,2-dioxaborolane (76 mg; 0.493 mmol; 2.00 eq.), Pd(DtBPF)Cl₂ (16 mg; 0.025 mmol; 0.10 eq.) and K₃PO₄ (105 mg; 0.493 mmol; 2.00 eq.). The reaction was stirred under nitrogen at 90° C. for 2 h. Upon completion, the reaction was concentrated under reduced pressure. The residue was purified by flash chromatography on silica gel column using EtOAc/CH₂Cl₂ (10-30%) to afford N-(5-phenyl-8-vinyl-2,7-naphthyridin-3-yl)cyclopropanecarboxamide as a light yellow solid (60 mg, 77%). LCMS (ESI) m/z 316.1, [M+H]⁺.

### Step 2: N-(8-ethyl-5-phenyl-2,7-naphthyridin-3-yl)cyclopropanecarboxamide

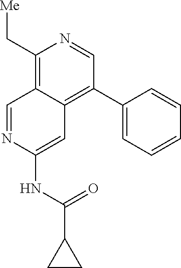 [SMILES: CCc1ncc(-c2ccccc2)c2cc(NC(=O)C3CC3)ncc12]

To a stirring mixture of N-(5-phenyl-8-vinyl-2,7-naphthyridin-3-yl)cyclopropanecarboxamide (50 mg; 0.159 mmol; 1.00 eq.) in MeOH (15 mL) was added 10% Pd/C (50 mg; 100% w/w). The reaction was stirred at 40° C. for 4 h under hydrogen atmosphere (2 atm). The resulting mixture was filtered, the filter cake was washed with a mixture solvent of CH₂Cl₂/MeOH (1:1, 50 mL). The filtrate was concentrated under reduced pressure. The crude product was purified by flash chromatography on pre-packed C18 column using 10-50% of MeCN in water (10 mmol/L NH₄HCO₃) to afford N-(8-ethyl-5-phenyl-2,7-naphthyridin-3-yl)cyclopropanecarboxamide as a off-white solid (12.5 mg, 24%). LCMS (ESI) m/z 318.2, [M+H]⁺, ¹H NMR (400 MHz, DMSO-d) δ 11.13 (s, 1H), 9.58 (s, 1H), 8.50 (s, 1H), 8.41 (s, 1H), 7.59-7.47 (m, 5H), 3.41 (q, J=7.2 Hz, 2H), 2.09-2.01 (m, 1H), 1.41 (t, J=7.2 Hz, 3H), 0.82-0.78 (m, 4H).

### Example 97: Synthesis of N¹-methyl-N⁶-(oxetan-3-yl)-4-phenyl-2,7-naphthyridine-1,6-diamine

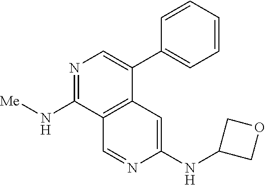 [SMILES: CNc1ncc(-c2ccccc2)c2cc(NC3COC3)ncc12]

### Step 1: 6-chloro-N-methyl-2,7-naphthyridin-1-amine

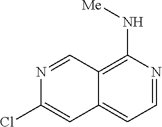 [SMILES: CNc1nccc2cc(Cl)ncc12]

To a stirring mixture of 6-chloro-2,7-naphthyridin-1(2H)-one (Example 2, step 3) (1 g; 5.53 mmol; 1.00 eq.) and PyBOP (5.7 g; 10.95 mmol, 2.00 eq.) in DMA (15 mL) were added methyl amine hydrochloride salt (1.11 g; 16.44 mmol; 3.00 eq.), DIEA (3.6 g; 27.85 mmol; 5.00 eq.). After stirring for 16 h at 80° C. under a nitrogen atmosphere, the reaction was cooled to rt. The resulting mixture was diluted with EtOAc (150 mL). The resulting solution was washed with a sat. NaCl solution (5×10 mL). The EtOAc was concentrated under vacuum. The residue was purified by flash chromatography on silica gel column using 10-40% of EtOAc in CH₂Cl₂ as eluent to provide 1.5 g of the crude product. The crude product was purified by flash chromatography on pre-packed C18 column using 20-50% of MeCN in water (10 mmol/L NH₄HCO₃) as eluent to provide 6-chloro-N-methyl-2,7-naphthyridin-1-amine as an off-white solid (676 mg, 63%). LCMS (ESI) m/z 194.0, [M+H]⁺.

### Step 2: 6-chloro-4-iodo-N-methyl-2,7-naphthyridin-1-amine

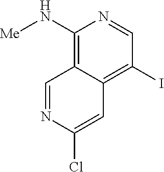 [SMILES: CNc1ncc(I)c2cc(Cl)ncc12]

To a solution of 6-chloro-N-methyl-2,7-naphthyridin-1-amine (612 mg; 3.16 mmol; 1.00 eq.) in DMF (12 mL) was added NIS (1.07 g; 4.75 mmol; 1.50 eq.) at 0° C. slowly. The mixture was stirred for 1 h at room temperature. The resulting mixture was diluted with EtOAc (150 mL) and then washed with a sat. NaCl solution (5×10 mL). The resulting solution (EtOAc) was dried with Na₂SO₄, and concentrated under vacuum. The residue was diluted with CH₂Cl₂ (15 mL) to precipitate the solids. The solids were collected by filtration and washed with CH₂Cl₂ to afford 6-chloro-4-iodo-N-methyl-2,7-naphthyridin-1-amine as a yellow solid (983 mg, 97%), LCMS (ESI) m/z 319.9, [M+H]⁺.

### Step 3: 6-chloro-N-methyl-4-phenyl-2,7-naphthyridin-1-amine

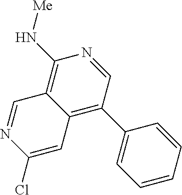 [SMILES: CNc1ncc(-c2ccccc2)c2cc(Cl)ncc12]

To a mixture of 6-chloro-4-iodo-N-methyl-2,7-naphthyridin-1-amine (950 mg, 2.97 mmol; 1.00 eq.) and phenylboronic acid (363 mg; 2.97 mmol; 1.00 eq.) in dioxane/water (10:1, 13.2 mL) were added K₃PO₄ (1.26 g; 5933 mmol; 2.00 eq.) and Pd(dppf)Cl₂·CH₂Cl₂ (242 mg; 0.297 mmol; 0.10 eq.). After stirring for 2 h at 60° C. under a nitrogen atmosphere, the reaction was cooled to rt. The resulting mixture was concentrated under reduced pressure. The residue was purified by flash chromatography on silica gel column using 20-50% of EtOAc in petroleum ether as eluent to provide 6-chloro-N-methyl-4-phenyl-2,7-naphthyridin-1-amine as a yellow solid (620 mg, 77%). LCMS (ESI) m/z 270.1, [M+H]⁺. ¹H NMR (400 MHz, DMSO-d₆) δ 9.45 (s, 1H), 8.32-8.21 (m, 1H), 8.10 (s, 1H), 7.60-7.50 (m, 2H), 7.49-7.38 (m, 4H), 3.04 (d, J=4.4 Hz, 3H).

### Step 4: N¹-methyl-N⁶-(oxetan-3-yl)-4-phenyl-2,7-naphthyridine-1,6-diamine

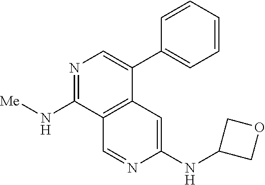 [SMILES: CNc1ncc(-c2ccccc2)c2cc(NC3COC3)ncc12]

A mixture of 6-chloro-N-methyl-4-phenyl-2,7-naphthyridin-1-amine (40 mg; 0.148 mmol; 1.00 e q), Pd-PEPPSI-IHeptCl (CAS: 1814936-54-3) (14.4 mg; 0.015 mmol; 0.10 eq.), t-BuOK. (33.3 m g; 0.297 mmol; 2.00 eq.) and oxetan-3-amine (10.8 mg; 0.148 mmol; 1.00 eq.) in dioxane (2 mL) was stirred at 90° C. for 2 hours. Upon completion, the resulting mixture was concentrated under vacuum. The residue was purified by flash chromatography on silica gel column using MeOH/CH₂Cl₂ (1-9%) of as eluent to provide N¹-methyl-N⁶-(oxetan-3-yl)-4-phenyl-2,7-naphthyridine-1,6-diamine (20 mg, crude). The crude product was purified by flash chromatography on pre-packed C18 column using 10%-70% of MeCN in water (10 mmol/L NH₄HCO₃) to provide N¹-methyl-N⁶-(oxetan-3-yl)-4-phenyl-2,7-naphthyridine-1,6-diamine as an orange solid (6.3 mg, 13%). LCMS (ESI) m/z 307.1, [M+H]⁺. ¹H NMR (400 MHz, DMSO-d₆) δ 9.11 (s, 1H), 7.71 (s, 1H), 7.69-7.6 4 (m, 1H), 7.53-7.45 (m, 3H), 7.42-7.35 (m, 3H), 6.41 (s, 1H), 4.95-4.85 (m, 1H), 4.79-4.73 (m, 2H), 4.45-4.40 (m, 2H), 2.96 (d, J=4.4 Hz, 3H).

Examples 98-100: Each compound in Table 4 was prepared using a similar experimental procedure to prepare Example 97 using 6-chloro-N-methyl-4-phenyl-2,7-naphthyridin-1-amine as the common intermediate and appropriate amines:

TABLE 4

| Example | Structure and Name | MS and ¹H NMR |
|---|---|---|
| Example 98 | 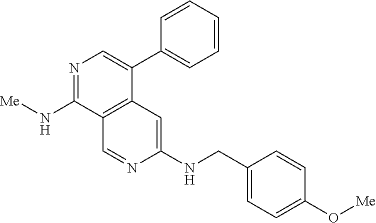 [SMILES: CNc1ncc(-c2ccccc2)c2cc(NCc3ccc(OC)cc3)ncc12] | LCMS (ESI) m/z 371.2, [M + H]⁺. ¹H NMR (400 MHz, DMSO-d₆) δ 9.10 (s, 1H), 7.68 (s, 1H), 7.66-7.60 (m, 1H), 7.47-7.40 (m, 2H), 7.39-7.26 (m, 4H), 7.18 (d, J = 8.4 Hz, 2H), 6.86 (d, J = 8.4 Hz, 2H), 6.37 (s, 1H), 4.35 (d, J = 5.6 Hz, 2H), 3.72 (s, 3H), 2.95 (d, J = 4.0 Hz, 3H). |
| | N⁶-(4-methoxybenzyl)-N¹-methyl- | |
| | 4-phenyl-2,7-naphthyridine-1,6- | |
| | diamine | |
| Example 99 | 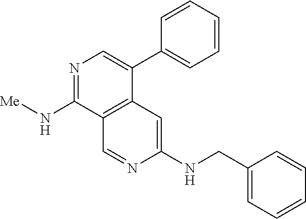 [SMILES: CNc1ncc(-c2ccccc2)c2cc(NCc3ccccc3)ncc12] | LCMS (ESI) m/z 341.2, [M + H]⁺. ¹H NMR (400 MHz, DMSO-d₆) δ 9.10 (s, 1H), 7.68 (s, 1H), 7.65-7.59 (m, 1H), 7.45-7.18 (m, 11H), 6.38 (s, 1H), 4.43 (d, J = 6.0 Hz, 2H), 2.95 (d, J = 4.4 Hz, 3H). |
| | N⁶-benzyl-N¹-methyl-4-phenyl- | |
| | 2,7-naphthyridine-1,6-diamine | |
| Example 100 | 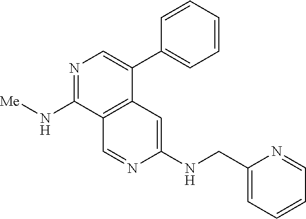 [SMILES: CNc1ncc(-c2ccccc2)c2cc(NCc3ccccn3)ncc12] | LCMS (ESI) m/z 342.2, [M + H]⁺. ¹H NMR (400 MHz, DMSO-d₆) δ 9.10 (s, 1H), 8.50 (d, J = 4.4 Hz, 1H), 7.79-7.60 (m, 3H), 7.51-7.40 (m, 3H), 7.38-7.32 (m, 1H), 7.31-7.20 (m, 4H), 6.43 (s, 1H), 4.53 (d, J = 5.6 Hz, 2H), 2.95 (d, J = 4.0 Hz, 3H). |
| | N¹-methyl-4-phenyl-N⁶-(pyridin- | |
| | 2-ylmethyl)-2,7-naphthyridine- | |
| | 1,6-diamine | |

### Example 101: Synthesis of 1-methyl-N-(8-(methylamino)-5-phenyl-2,7-naphthyridin-3-yl)cyclopropane-1-carboxamide

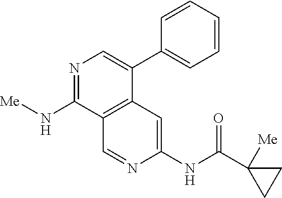 [SMILES: CNc1ncc(-c2ccccc2)c2cc(NC(=O)C3(C)CC3)ncc12]

To a stirring mixture of 6-chloro-N-methyl-4-phenyl-2,7-naphthyridin-1-amine (Example 97, Step 3) (30 mg; 0.111 mmol; 1.00 eq.) was added to 1-methylcyclopropane-1-carboxamide (21.9 mg; 0.221 mmol; 2.00 eq.), Pd₂(dba)₃ (10.2 mg; 0.011 mmol; 0.10 eq.), XantPhos (12.9 mg; 0.022 mmol; 0.20 eq.) and Cs₂CO₃ (72.1 mg; 0.221 mmol; 1.99 eq.) in 1,4-dioxane (2 mL) under nitrogen atmosphere. The reaction was stirred at 110° C. for 3 h. The desired product was observed via. LCMS. The reaction mixture was concentrated in vacuo. The residue was purified by flash chromatography on silica gel column using MeOH/CH₂Cl₂ (0-6%) to give the crude product. The crude product was further purified by flash chromatography on pre-packed C18 column using 20-55% of MeCN in water (10 mmol/L NH₄CO₃) to afford 1-methyl-N-(8-(methylamino)-5-phenyl-2,7-naphthyridin-3-yl)cyclopropane-1-carboxamide as an off-white solid (24.4 mg, 66%). LCMS (ESI) m/z 333.2, [M+H]⁺. ¹H NMR (400 MHz, DMSO-d₆) δ 9.59 (s, 1H), 9.40 (s, 1H), 8.31 (s, 1H), 8.03-7.98 (m, 1H), 7.94 (s, 1H), 7.52-7.36 (m, 5H), 3.02 (d, J=4.4 Hz, 3H), 1.41 (s, 3H), 1.11-1.07 (m, 2H), 0.65-0.61 (m, 2H)

### Example 102 was prepared using a similar experimental procedure to prepare Example 97

| Example | Structure and Name | MS and ¹H NMR |
|---|---|---|
| Example 102 | 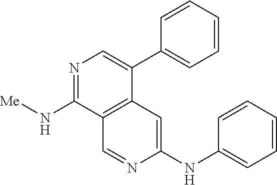 [SMILES: CNc1ncc(-c2ccccc2)c2cc(Nc3ccccc3)ncc12] | LCMS (ESI) m/z 327.2, [M + H]⁺. ¹H NMR (400 MHz, DMSO-d₆) δ 9.27 (s, 1H), 9.21 (s, 1H), 7.87-7.79 (m, 2H), 7.59-7.54 (m, 2H), 7.53-7.48 (m, 2H), 7.47-7.42 (m, 2H), 7.41- 7.37 (m, 1H), 7.31-7.22 (m, 2H), 6.95-6.86 (m, 2H), 3.01 (d, J = 4.4 Hz, 3H). |
| | N⁶-methyl-N⁶,4-diphenyl-2,7- | |
| | naphthyridine-1,6-diamine | |

Example 103 and Example 104: Synthesis of 2-fluoro-N-(8-(methylamino)-5-phenyl-2,7-naphthyridin-3-yl)cyclopropane-1-carboxamide (trans isomer: 103, cis isomer: 104)

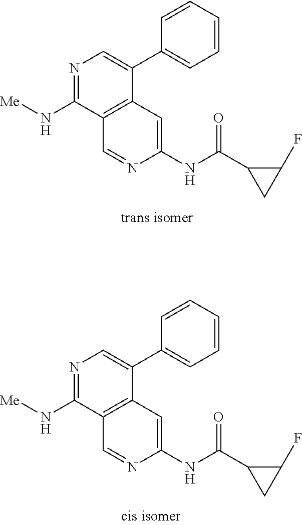 [SMILES: CNc1ncc(-c2ccccc2)c2cc(NC(=O)C3CC3F)ncc12 ; CNc1ncc(-c2ccccc2)c2cc(NC(=O)C3CC3F)ncc12]

### Step 1: 2-fluorocyclopropane-1-carboxamide

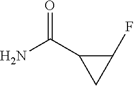

To a stirred solution of 2-fluorocyclopropane-1-carboxylic acid (200 mg; 1.92 mmol, 1.00 eq.) and DMF (28 mg; 0.383 mmol; 0.20 eq.) in CH₂Cl₂ (6 mL) was added dropwise a solution of oxalyl chloride (2 M) in CH₂Cl₂ (1.15 mL, 2.3 mmol; 1.20 eq.) at 0° C. The resulting mixture was stirred for 1 h at room temperature under nitrogen atmosphere. Upon completion, the resulting mixture was concentrated under reduced pressure. This crude product was added to a stirred solution of NH₃(g) in MeOH (7 M, 3 mL) dropwise at 0° C. The resulting mixture was stirred for 1 h at room temperature. The desired product was Observed by LCMS. The resulting mixture was concentrated under reduced pressure. The residue was purified by flash chromatography on silica gel column using MeOH/CH₂Cl₂ (1%-9%) as eluent to provide 2-fluorocyclopropane-1-carboxamide as a off-white solid (40 mg, 20%). LCMS (ESI) m/z 104.0, [M+H]⁺.

### Step 2: 2-fluoro-N-(8-(methylamino)-5-phenyl-2,7-naphthyridin-3-cyclopropane-1-carboxamide (cis isomer and trans isomer)

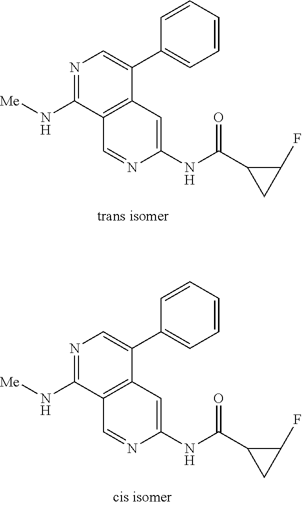 [SMILES: CNc1ncc(-c2ccccc2)c2cc(NC(=O)C3CC3F)ncc12 ; CNc1ncc(-c2ccccc2)c2cc(NC(=O)C3CC3F)ncc12]

To a stirring mixture of 6-chloro-N-methyl-4-phenyl-2,7-naphthyridin-1-amine (Example 97, step 3) (40 mg; 0.148 mmol; 1.00 eq.) in dioxane (2 mL) was added Pd₂(dba)₃ (13.6 mg; 0.015 mmol 0.10 eq.), XantPhos (17.2 mg; 0.030 mmol; 0.20 eq.), Cs₂CO₃ (96.6 mg; 0.296 mmol; 2.00 eq.) and 2-fluorocyclopropane-1-carboxamide (23 mg; 0.223 mmol; 1.50 eq.) at room temperature under nitrogen atmosphere. The reaction was stirred at 110° C. for 3 h. The desired product was detected by LCMS. The resulting mixture was concentrated under vacuum. The residue was purified by flash chromatography on silica gel column using MeOH/CH₂Cl₂ (1-9%) as eluent to provide two separated peaks with desired product mass. The less polar peak was the minor component (˜10 mg) compared to the more polar peak (30 mg). The two peaks were further purified separately by flash chromatography on pre-packed C18 column using 10%-50% of MeCN in water (10 mmol/L NH₄HCO₃) to provide 2-fluoro-N-(8-(methylamino)-5-phenyl-2,7-naphthyridin-3-yl)cyclopropane-1-carboxamide as a white solid (trans isomer) (3.8 mg, 7%) and 2-fluoro-N-(8-(methylamino)-5-phenyl-2,7-naphthyridin-3-yl)cyclopropane-1-carboxamide as a white solid (cis isomer) (21.5 mg, 43%). LCMS (ESI) m/z 337.1, [M+H]⁺. HNMR for trans isomer: ¹H NMR (400 MHz, DMSO-d₆) δ 11.09 (s, 1H), 9.39 (s, 1H), 8.30 (s, 1H), 8.01-7.96 (m, 1H), 7.94 (s, 1H), 7.50-7.36 (m, 5H), 4.96-4.75 (m, 1H), 3.01 (d, J=4.4 Hz, 3H), 2.60-2.54 (m, 1H), 1.54-1.42 (m, 1H), 1.26-1.16 (m, 1H). HNMR for cis isomer: ¹H NMR (400 MHz, DMSO-d₆) δ 10.97 (s, 1H), 9.39 (s, 1H), 8.34 (s, 1H), 8.01-7.96 (m, 1H), 7.94 (s, 1H), 7.53-7.37 (m, 5H), 5.01-4.78 (m, 1H), 3.02 (d, J=4.4 Hz, 3H), 2.25-2.16 (m, 1H), 1.66-1.53 (m, 1H), 1.18-1.08 (m, 1H).

### Example 105: Synthesis of 2,2-difluoro-N-(8-(methylamino)-5-phenyl-2,7-naphthyridin-3-yl)cyclopropane-1-carboxamide

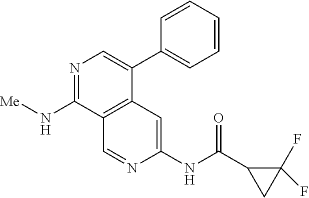 [SMILES: CNc1ncc(-c2ccccc2)c2cc(NC(=O)C3CC3(F)F)ncc12]

### Step 1: N-(2,4-dimethoxybenzyl)-2,2-difluorocyclopropane-1-carboxamide

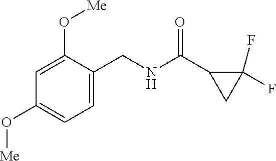 [SMILES: COc1ccc(CNC(=O)C2CC2(F)F)c(OC)c1]

A mixture of 2,2-difluorocyclopropane-1-carboxylic acid (200 mg; 1.63 mmol; 1.00 eq.) and HATU (741 mg; 1.94 mmol; 1.19 eq.) in DMF (2 mL) was stirred for 20 min at room temperature under nitrogen atmosphere. To this reaction mixture was added DIPEA (629 mg; 4.86 mmol; 3.00 eq.) and (2,4-dimethoxyphenyl)methanamine (543 mg; 3.24 mmol; 1.98 eq.). The resulting mixture was stirred for an additional 2 h at room temperature. The reaction was monitored by LCMS. The residue was purified by flash chromatography on pre-packed C18 column using 20%-60% of MeCN in water (10 mmol/L NH₄HCO₃) to provide N-(2,4-dimethoxybenzyl)-2,2-difluorocyclopropane-1-carboxamide as a yellow solid (393 mg, 88%). LC-MS: (ESI, m/z): [M+H]⁺=272.1.

### Step 2: 2,2-difluorocyclopropane-1-carboxamide

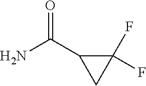

A mixture of N-(2,4-dimethoxybenzyl)-2,2-difluorocyclopropane-1-carboxamide (393 mg; 1.44 mmol; 1.00 eq.) in trifluoroacetaldehyde (3 mL) was stirred for 1 h at 70° C. under nitrogen atmosphere. The reaction was monitored by LCMS. The resulting mixture was concentrated under reduced pressure. The residue was purified by flash chromatography on silica gel column using 2-6% of MeOH in CH₂Cl₂ as eluent to provide 2,2-difluorocyclopropane-1-carboxamide as a white solid (170 mg, 97%). LCMS (ESI) m/z 122.0, [M+H]⁺.

### Step 3: 2-difluoro-N-(8-(methylamino)-5-phenyl-2,7-naphthyridin-3-yl)cyclopropane-carboxamide

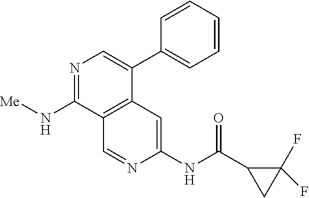 [SMILES: CNc1ncc(-c2ccccc2)c2cc(NC(=O)C3CC3(F)F)ncc12]

To a solution of 6-chloro-N-methyl-4-phenyl-2,7-naphthyridin-1-amine (Example 97, step 3) (50 mg; 0.185 mmol; 1.00 eq.) and 2,2-difluorocyclopropane-1-carboxamide (27 mg; 0.223 mmol; 1.20 eq.) in dioxane (4 mL) and Cs₂CO₃ (121.6 mg; 0.373 mmol, 2.00 eq.) was added Pd₂(dba)₃ (14 mg; 0.015 mmol; 0.10 eq.) and XantPhos (17 mg; 0.02 mmol; 0.20 eq.). After stirring for 3 h at 110° C. under a nitrogen atmosphere. The reaction was monitored by LCMS. Upon completion, the resulting mixture was concentrated under reduced pressure. The residue was purified by flash chromatography on silica gel column using 2-6% of MeOH in CH₂Cl₂ as eluent to provide 2,2-difluoro-N-(8-(methylamino)-5-phenyl-2,7-naphthyridin-3-yl)cyclopropane-1-carboxamide as a yellow solid (50 mg, crude). The residue was purified by flash chromatography on pre-packed C18 column using 20%-60% of MeCN in water (10 mmol/L NH₄HCO₃) to provide 2,2-difluoro-N-(8-(methylamino)-5-phenyl-2,7-naphthyridin-3-yl)cyclopropane-1-carboxamide as a white solid (24.5 mg, 46%). LCMS (ESI) m/z 355.1, [M+H]⁺. ¹H NMR (400 MHz, DMSO-d₆) δ 11.15 (s, 1H), 9.41 (s, 1H), 8.33 (s, 1H), 8.04-7.99 (m, 1H), 7.96 (s, 1H), 7.53-7.38 (m, 5H), 3.02 (d, J=4.4 Hz, 3H), 3.00-2.94 (m, 1H), 2.05-1.91 (m, 2H).

### Example 106: Synthesis of N-(8-(methylamino)-5-(1H-pyrrolo[2,3-b]pyridin-2,7-naphthyridin-3-yl)cyclopropanecarboxamide

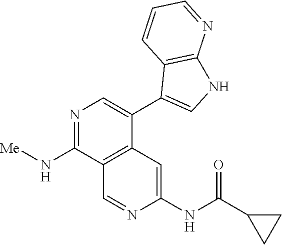 [SMILES: CNc1ncc(-c2c[nH]c3ncccc23)c2cc(NC(=O)C3CC3)ncc12]

A mixture of K₃PO₄(53 mg; 0.250 mmol; 2.00 eq.), N-(5-bromo-8-(methylamino)-2,7-naphthyridin-3-yl)cyclopropanecarboxamide (40 mg; 0.125 mmol; 1.00 eq.), 3-(4,4,5,5-tetramethyl-1,3,2-dioxaborolan-2-yl)-1H-pyrrolo[2,3-b]pyridine (36.6 mg; 0.150 mmol; 1.20 eq.) and 1,1′-bis(di-tert-butylphosphino)ferrocene palladium dichloride (8.1 mg; 0.012 mmol; 0.10 eq.) in 1,4-dioxane/water (5:1, 2.4 mL) was stirred at 90° C. for 2 h under nitrogen atmosphere. The mixture was allowed to cool down to room temperature and the solvent was concentrated under reduced pressure. The residue was purified by flash chromatography on silica gel column using MeOH/CH₂Cl₂ (1-10%) as eluent to provide the crude product. The crude product was further purified by flash chromatography on pre-packed C18 column using 20-50% MeCN in water (10 mmol/L NH₄HCO₃) as eluent to provide N-(8-(methylamino)-5-(1H-pyrrolo[2,3-b]pyridin-3-yl)-2,7-naphthyridin-3-yl)cyclopropanecarboxamide as a white solid (23.5 mg, 52%). LCMS (ESI) m/z 359.2, [M+H]⁺. ¹H NMR (400 MHz, DMSO-d₆) 110.88 (5, 1H), 10.92 (s, 1H), 9.41 (s, 1H), 8.36 (s, 1H), 8.28 (dd, J=4.8, 1.2 Hz, 1H), 8.10-7.96 (m, 2H), 7.75 (dd, J=4.8, 1.2 Hz, 1H), 7.58 (d, J=2.4 Hz, 1H), 7.06 (dd, J=8.0, 4.8 Hz, 1H), 3.04 (d, J=4.4 Hz, 3H), 2.05-1.94 (m, 1H), 0.81-0.68 (m, 4H).

### Examples 107-119

Each compound in Table 5 was prepared using a similar experimental procedure to prepare Example 106, using N-(5-bromo-8-(methylamino)-2,7-naphthyridin-3-yl)cyclopropanecarboxamide as the common intermediate and appropriate boronic ester or acid)

TABLE 5

| Example | Structure and Name | MS and ¹H NMR |
|---|---|---|
| Example 107 | 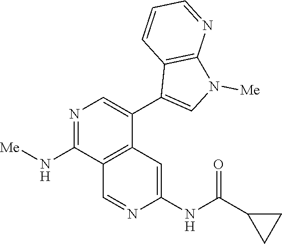 [SMILES: CNc1ncc(-c2cn(C)c3ncccc23)c2cc(NC(=O)C3CC3)ncc12] | LCMS (ESI) m/z 373.2, [M + H]⁺. ¹H NMR (400 MHz, DMSO-d₆) δ 10.89 (s, 1H), 9.40 (s, 1H), 8.33 (dd, J = 4.8, 1.6 Hz, 1H), 8.28 (s, 1H), 8.00 (s, 1H), 7.98- 7.90 (m, 1H), 7.71 (dd, J = 7.8, 1.6 Hz, 1H), 7.64 (s, 1H), 7.08 (dd, J = 7.8, 4.6 Hz, 1H), 3.92 (s, 3H), 3.03 (d, J = 4.4 Hz, 3H), 2.05-1.96 (m, 1H), 0.75-0.69 (m, 4H). |
| | N-(5-(1-methyl-1H- | |
| | pyrrolo[2,3-b]pyridin-3-yl)-8- | |
| | (methylamino)-2,7- | |
| | naphthyridin-3- | |
| | yl)cyclopropanecarboxamide | |
| Example 108 | 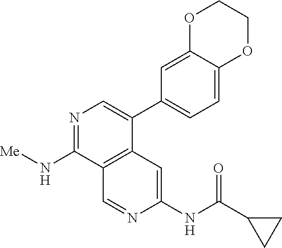 [SMILES: CNc1ncc(-c2ccc3c(c2)OCCO3)c2cc(NC(=O)C3CC3)ncc12] | LCMS (ESI) m/z 377.2, [M + H]⁺. ¹H NMR (400 MHz, DMSO-d₆) δ 10.91 (s, 1H), 9.36 (s, 1H), 8.32 (s, 1H), 7.95-7.89 (m, 1H), 7.87 (s, 1H), 6.95 (d, J = 8.0 Hz, 1H), 6.91-6.80 (m, 2H), 4.30 (s, 4H), 3.00 (d, J = 4.4 Hz, 3H), 2.08-1.98 (m, 1H), 0.85-0.75 (m, 4H). |
| | N-(5-(2,3- | |
| | dihydrobenzo[b][1,4]dioxin-6- | |
| | yl)-8-(methylamino)-2,7- | |
| | naphthyridin-3- | |
| | yl)cyclopropanecarboxamide | |
| Example 109 | 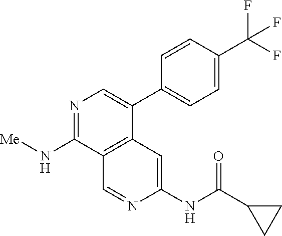 [SMILES: CNc1ncc(-c2ccc(C(F)(F)F)cc2)c2cc(NC(=O)C3CC3)ncc12] | LCMS (ESI) m/z 387.1, [M + H]⁺. ¹H NMR (400 MHz, DMSO-d₆) δ 10.99 (s, 1H), 9.40 (s, 1H), 8.34 (s, 1H), 8.13- 8.05 (m, 1H), 7.99 (s, 1H), 7.87-7.80 (m, 2H), 7.69-7.61 (m, 2H), 3.02 (d, J = 4.4 Hz, 3H), 2.06-1.97 (m, 1H), 0.83-0.73 (m, 4H). |
| | N-(8-(methylamino)-5-(4- | |
| | (trifluoromethyl)phenyl)-2,7- | |
| | naphthyridin-3- | |
| | yl)cyclopropanecarboxamide | |
| Example 110 | 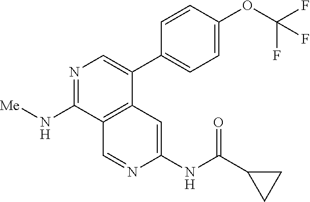 [SMILES: CNc1ncc(-c2ccc(OC(F)(F)F)cc2)c2cc(NC(=O)C3CC3)ncc12] | LCMS (ESI) m/z 403.1, [M + H]⁺. ¹H NMR (400 MHz, DMSO-d₆) δ 10.96 (s, 1H), 9.39 (s, 1H), 8.31 (s, 1H), 8.11- 8.00 (m, 1H), 7.96 (s, 1H), 7.65-7.40 (m, 4H), 3.02 (d, J = 4.4 Hz, 3H), 2.11-1.95 (m, 1H), 0.92-0.70 (m, 4H). |
| | N-(8-(methylamino)-5-(4- | |
| | (trifluoromethoxy)phenyl)-2,7- | |
| | naphthyridin-3- | |
| | yl)cyclopropanecarboxamide | |
| Example 111 | 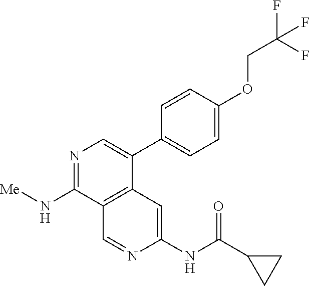 [SMILES: CNc1ncc(-c2ccc(OCC(F)(F)F)cc2)c2cc(NC(=O)C3CC3)ncc12] | LCMS (ESI) m/z 417.1, [M + H]⁺. ¹H NMR (400 MHz, DMSO-d₆) δ 10.93 (s, 1H), 9.37 (s, 1H), 8.31 (s, 1H), 7.99- 7.91 (m, 1H), 7.89 (s, 1H), 7.40-7.34 (m, 2H), 7.20-7.14 (m, 2H), 4.88-4.77 (m, 2H), 3.00 (d, J = 4.4 Hz, 3H), 2.07- 1.96 (m, 1H), 0.82-0.74 (m, 4H). |
| | N-(8-(methylamino)-5-(4- | |
| | (2,2,2-trifluoroethoxy)phenyl)- | |
| | 2,7-naphthyridin-3- | |
| | yl)cyclopropanecarboxamide | |
| Example 112 | 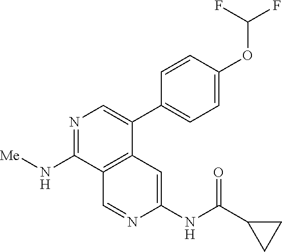 [SMILES: CNc1ncc(-c2ccc(OC(F)F)cc2)c2cc(NC(=O)C3CC3)ncc12] | LCMS (ESI) m/z 385.1, [M + H]⁺. ¹H NMR (400 MHz, DMSO-d₆) δ 10.96 (s, 1H), 9.38 (s, 1H), 8.31 (s, 1H), 8.04-7.96 (m, 1H), 7.92 (s, 1H), 7.48-7.43 (m, 2H), 7.33 (t, J = 74.0 Hz, 1H), 7.31- 7.27 (m, 2H), 3.01 (d, J = 4.4 Hz, 3H), 2.05-1.97 (m, 1H), 0.81-0.76 (m, 4H). |
| | N-(5-(4- | |
| | (difluoromethoxy)phenyl)-8- | |
| | (methylamino)-2,7- | |
| | naphthyridin-3- | |
| | yl)cyclopropanecarboxamide | |
| Example 113 | 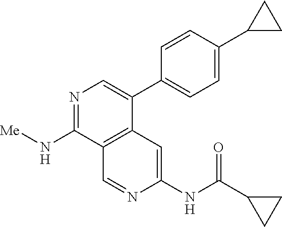 [SMILES: CNc1ncc(-c2ccc(C3CC3)cc2)c2cc(NC(=O)C3CC3)ncc12] | LCMS (ESI) m/z 359.2, [M + H]⁺. ¹H NMR (400 MHz, DMSO-d₆) δ 10.91 (s, 1H), 9.37 (s, 1H), 8.34 (s, 1H), 8.00- 7.91 (m, 1H), 7.89 (s, 1H), 7.28 (d, J = 8.0 Hz, 2H), 7.18 (d, J = 8.0 Hz, 2H), 3.01 (d, J = 4.4 Hz, 3H), 2.07-1.93 (m, 2H), 1.04-0.95 (m, 2H), 0.88-0.70 (m, 6H). |
| | N-(5-(4-cyclopropylphenyl)-8- | |
| | (methylamino)-2,7- | |
| | naphthyridin-3- | |
| | yl)cyclopropanecarboxamide | |
| Example 114 | 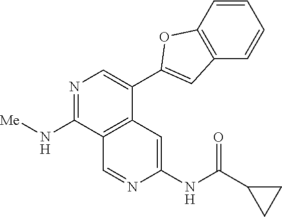 [SMILES: CNc1ncc(-c2cc3ccccc3o2)c2cc(NC(=O)C3CC3)ncc12] | LCMS (ESI) m/z 359.1, [M + H]⁺. ¹H NMR (400 MHz, DMSO-d₆) δ 11.07 (s, 1H), 9.42 (s, 1H), 8.81 (s, 1H), 8.45 (s, 1H), 8.34-8.29 (m, 1H), 7.72-7.58 (m, 2H), 7.36-7.24 (m, 2H), 7.08 (s, 1H), 3.05 (d, J = 4.4 Hz, 3H), 2.11-2.03 (m, 1H), 0.90-0.80 (m, 4H). |
| | N-(5-(benzofuran-2-yl)-8- | |
| | (methylamino)-2,7- | |
| | naphthyridin-3- | |
| | yl)cyclopropanecarboxamide | |
| Example 115 | 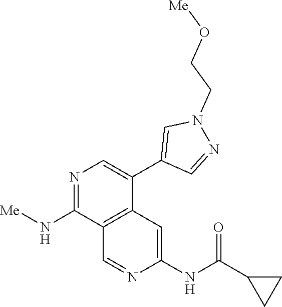 [SMILES: CNc1ncc(-c2cnn(CCOC)c2)c2cc(NC(=O)C3CC3)ncc12] | LCMS (ESI) m/z 367.2, [M + H]⁺. ¹H NMR (400 MHz, DMSO-d₆) δ 10.96 (s, 1H), 9.35 (s, 1H), 8.52 (s, 1H), 7.99 (s, 1H), 7.90 (s, 1H), 7.89-7.85 (m, 1H), 7.63 (s, 1H), 4.32 (t, J = 5.6 Hz, 2H), 3.75 (t, J = 5.6 Hz, 2H), 3.27 (s, 3H), 2.98 (d, J = 4.4 Hz, 3H), 2.08-2.00 (m, 1H), 0.86-0.78 (m, 4H). |
| | N-(5-(1-(2-methoxyethyl)-1H- | |
| | pyrazol-4-yl)-8-(methylamino)- | |
| | 2,7-naphthyridin-3- | |
| | yl)cyclopropanecarboxamide | |
| Example 116 | 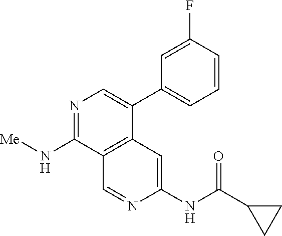 [SMILES: CNc1ncc(-c2cccc(F)c2)c2cc(NC(=O)C3CC3)ncc12] | LCMS (ESI) m/z 337.1, [M + H]⁺. ¹H NMR (400 MHz, DMSO-d₆) δ 10.95 (s, 1H), 9.39 (s, 1H), 8.34 (s, 1H), 8.05- 8.02 (m, 1H), 7.97 (s, 1H), 7.58-7.49 (m, 1H), 7.30-7.19 (m, 3H), 3.01 (d, J = 4.4 Hz, 3H), 2.06-1.96 (m, 1H), 0.82- 0.75 (m, 4H). |
| | N-(5-(3-fluorophenyl)-8- | |
| | (methylamino)-2,7- | |
| | naphthyridin-3- | |
| | yl)cyclopropanecarboxamide | |
| Example 117 | 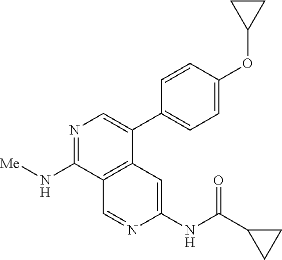 [SMILES: CNc1ncc(-c2ccc(OC3CC3)cc2)c2cc(NC(=O)C3CC3)ncc12] | LCMS (ESI) m/z 375.2, [M + H]⁺. ¹H NMR (400 MHz, DMSO-d₆) δ 10.93 (s, 1H), 9.37 (s, 1H), 8.33 (s, 1H), 7.98- 7.90 (m, 1H), 7.89 (s, 1H), 7.33 (d, J = 8.4 Hz, 2H), 7.16 (d, J = 8.4 Hz, 2H), 3.92-3.87 (m, 1H), 3.00 (d, J = 4.4 Hz, 3H), 2.10-1.98 (m, 1H), 0.90-0.71 (m, 8H). |
| | N-(5-(4-cyclopropoxyphenyl)- | |
| | 8-(methylamino)-2,7- | |
| | naphthyridin-3-yl) | |
| | cyclopropanecarboxamide | |
| Example 118 | 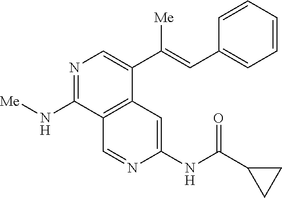 [SMILES: CNc1ncc(/C(C)=C/c2ccccc2)c2cc(NC(=O)C3CC3)ncc12] | LCMS (ESI) m/z 359.2, [M + H]⁺. ¹H NMR (400 MHz, DMSO-d₆) δ 10.97 (s, 1H), 9.35 (s, 1H), 8.38 (s, 1H), 7.93 (s, 1H), 7.92-7.87 (m, 1H), 7.47-7.38 (m, 4H), 7.33-7.24 (m, 1H), 6.53 (s, 1H), 2.98 (d, J = 4.4 Hz, 3H), 2.22 (s, 3H), 2.07-2.00 (m, 1H), 0.85-0.75 (m, 4H). |
| | (E)-N-(8-(methylamino)-5-(1- | |
| | phenylprop-1-en-2-yl)-2,7- | |
| | naphthyridin-3- | |
| | yl)cyclopropanecarboxamide | |
| Example 119 | 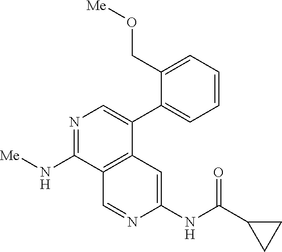 [SMILES: CNc1ncc(-c2ccccc2COC)c2cc(NC(=O)C3CC3)ncc12] | LCMS (ESI) m/z 363.2, [M + H]⁺. ¹H NMR (400 MHz, DMSO-d₆) δ 10.88 (s, 1H), 9.37 (s, 1H), 7.98-7.95 (m, 1H), 7.82 (s, 1H), 7.78 (s, 1H), 7.58-7.50 (m, 1H), 7.48-7.34 (m, 2H), 7.23-7.16 (m, 1H), 4.20-4.08 (m, 2H), 3.10 (s, 3H), 3.01 (d, J = 4.4 Hz, 3H), 2.02-1.94 (m, 1H), 0.80-0.68 (m, 4H). |
| | N-(5-(2- | |
| | (methoxymethyl)phenyl)-8- | |
| | (methylamino)-2,7- | |
| | naphthyridin-3- | |
| | yl)cyclopropanecarboxamide | |

### Example 120: Synthesis of 6-(cyclopropanecarboxamido)-1-(methylamino)-2,7-naphthyridine-4-carboxylic acid

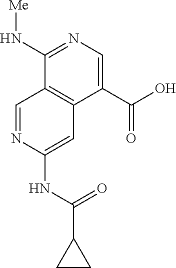 [SMILES: CNc1ncc(C(=O)O)c2cc(NC(=O)C3CC3)ncc12]

### Step 1: methyl 6-(cyclopropanecarboxamido)-1-(methylamino)-2,7-naphthyridine-4-carboxylate

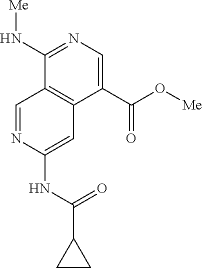 [SMILES: CNc1ncc(C(=O)OC)c2cc(NC(=O)C3CC3)ncc12]

To a solution of N-(5-bromo-8-(methylamino)-2,7-naphthyridin-3-yl)cyclopropanecarboxamide (200 mg: 0.623 mmol; 1.00 eq.) in a mixture solvent of DMSO/methanol. (2:1, 9 mL) was added Pd(dppf)Cl₂. CH₂Cl₂ (51 mg; 0.063 mmol; 0.10 eq.) and Et₃N (189 mg; 1.868 mmol; 3.00 eq.) in a pressure tank. The mixture was pressurized to 20 atm with carbon monoxide and stirred at 130° C. overnight. The reaction mixture was cooled to room temperature. Upon completion, the reaction solution was diluted with EtOAc (100 mL), washed with brine (3×20 mL), dried over Na₂SO₄. After filtration, the filtrate was concentrated under reduced pressure. The residue was purified by flash chromatography on silica gel column using 60-90% of EtOAc in CH₂Cl₂ as eluent to provide methyl 6-(cyclopropanecarboxamido)-1-(methylamino)-2,7-naphthyridine-4-carboxylate as a yellow solid (170 mg, 90%). LCMS (ESI) m/z 301.1, [M+H]⁺.

### Step 2: 6-(cyclopropanecarboxamido)-1-(methylamido-2,7-naphthyridine-4-carboxylic acid

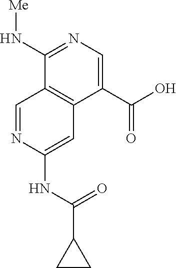 [SMILES: CNc1ncc(C(=O)O)c2cc(NC(=O)C3CC3)ncc12]

A mixture of methyl 6-(cyclopropanecarboxamido)-1-(methylamino)-2,7-naphthyridine-4-carboxylate (150 mg; 0.499 mmol; 1.00 eq.) and lithium hydroxide monohydrate (42 mg; 1.00 mmol; 2.00 eq.) in THF/water (3:1, 12 mL) was stirred overnight at room temperature under nitrogen atmosphere. Upon completion, the reaction mixture was concentrated under vacuum. The residue was dissolved in water (2 The mixture was acidified to pH 6 with 1 M ETC. The precipitated solids were collected by filtration and washed with water (2 The solids were dried under vacuum to afford 6-cyclopropaneamido-1-(methylamino)-2,7-naphthyridine-4-carboxylic acid as a white solid (140 mg, 97%). Then 10 mg of the crude product was further purified by flash chromatography on pre-packed C18 column using 10-60% of MeCN in water (0.05% formic acid) to provide 6-cyclopropaneamido-1-(methylamino)-2,7-naphthyridine-4-carboxylic acid as a white solid (2.9 mg). LCMS (ESI) m/z 287.1, [M+H]⁺. ¹H NMR (400 MHz, DMSO-d₆) δ 10.89 (s, 1H), 9.39 (s, 1H), 9.34 (s, 1H), 8.73 (s, 1H), 8.55-8.48 (m, 1H), 3.04 (d, J=4.4 Hz, 3H), 2.09-2.00 (m, 1H), 0.88-0.77 (m, 4H).

### Example 121: Synthesis of N-(8-(methylamino)-5-(4-phenoxypiperidine-1-carbonyl)-2,7-naphthyridin-3-yl)cyclopropanecarboxamide

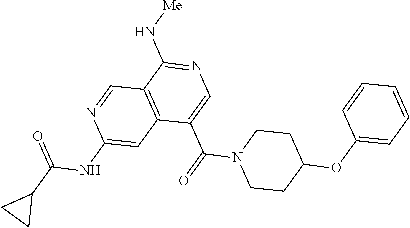 [SMILES: CNc1ncc(C(=O)N2CCC(Oc3ccccc3)CC2)c2cc(NC(=O)C3CC3)ncc12]

A mixture of 6-cyclopropaneamido-1-(methylamino)-2,7-naphthyridine-4-carboxylic acid (50 mg; 0.175 mmol; 1.00 eq.), 4-phenoxypiperidine (37.2 mg; 0.210 mmol; 1.20 eq.), HATU (100 mg; 0.263 mmol 1.51 eq.) and DIPEA (67.6 mg; 0.523 mmol; 2.99 eq.) in DMF (3 mL) was stirred for 2 h at room temperature. Upon completion, the reaction mixture was purified by flash chromatography on pre-packed C18 column using 20-50% of MeCN in water (10 mmol/L NH₄HCO₃) to provide N-(8-(methylamino)-5-(4-phenoxypiperidine-1-carbonyl)-2,7-naphthyridin-3-yl)cyclopropanecarboxamide as a white solid (59.1 mg, 75%). LCMS (ESI) m/z 446.2, [M+H]⁺. ¹H NMR (400 MHz, Methanol-d₄) δ 9.23 (s, 1H), 8.35 (s, 1H), 8.00 (s, 1H), 7.30-7.23 (m, 2H), 7.00-6.89 (m, 3H), 4.72-4.62 (m, 1H), 4.12-3.79 (m, 2H), 3.68-3.52 (m, 1H), 3.45-3.36 (m, 1H), 3.10 (s, 3H), 2.24-1.63 (m, 5H), 1.10-0.88 (m, 4H).

### Example 122: Synthesis of N-(8-(methylamino)-5-(piperidine-1-carbonyl)-2,7-naphthyridin-3-yl)cyclopropanecarboxamide

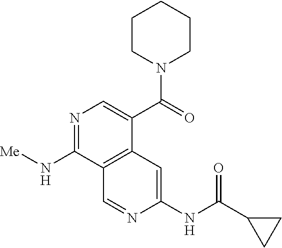 [SMILES: CNc1ncc(C(=O)N2CCCCC2)c2cc(NC(=O)C3CC3)ncc12]

A mixture of 6-(cyclopropanecarboxamido)-1-(methylamino)-2,7-naphthylidine-4-carboxylic acid (50 mg; 0.175 mmol; 1.00 eq.), piperidine (17.9 mg; 0.210 mmol; 1.20 eq.), HAITI (100 mg; 0.263 mmol; 1.50 eq.) and DIPEA (67.6 mg; 0.523 mmol; 3.00 eq.) in DMF (3 mL) was stirred for 1 h at room temperature. The desired product was observed via LCMS. The reaction mixture was purified by flash chromatography on pre-packed C18 column using 20-50% of MeCN in water (10 mmol/L, —NH₄HCO₃) to provide N-(8-(methylamino)-5-(piperidine-1-carbonyl)-2,7-naphthyridin-3-yl)cyclopropanecarboxamide as a white solid (42.6 mg, 69%). LCMS (ESI) m/z 354.2, [M+H]⁺. ¹H NMR (400 MHz, Methanol-d₄) δ 9.23 (s, 1H), 8.30 (s, 1H), 7.95 (s, 1.14), 3.86-3.74 (m, 2H), 3.39-3.34 (m, 2H), 3.10 (s, 3H), 1.97-1.90 (m, 1H), 1.81-1.44 (m, 6H), 1.04-0.89 (m, 4H).

### Example 123: Synthesis of 6-(cyclopropanecarboxamido)-1-(methylamino)-2,7-naphthyridine-4-carboxamide

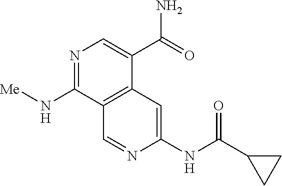 [SMILES: CNc1ncc(C(N)=O)c2cc(NC(=O)C3CC3)ncc12]

A mixture of 6-(cyclopropanecarboxamido)-1-(methylamino)-2,7-naphthyridine-4-carboxyli c acid (21.3 mg; 0.074 mmol; 1 eq.), NH₄Cl (19.7 mg; 0.371 mmol; 5.00 eq.), HATU (42.4 mg; 0.112 mmol; 1.50 eq.) and DIPEA (28.8 mg; 0.223 mmol; 3.00 eq.) in DMF (1.5 mL) was stirred for 1 h at room temperature. Upon completion, the reaction mixture was purified by flash chromatography on pre-packed C18 column using 20-50% of MeCN in water (10 mmol/L NH₄HCO₃) to provide 6-(cyclopropanecarboxamido)-1-(methylamino)-2,7-naphthyridine-4-carboxamide as a white solid (8.6 mg, 40%). LCMS (ESI) m/z 286.1, [M+H]⁺. ¹H NMR (400 MHz, DMSO-d₆) δ 10.86 (s, 1H), 9.32 (s, 1H), 8.96 (s, 1H), 8.31 (s, 1H), 8.24-8.19 (m, 1H), 7.67 (s, 1H), 7.10 (s, 1H), 3.00 (d, J=4.4 Hz, 3H), 2.09-2.01 (m, 1H), 0.90-0.78 (m, 4H).

### Example 124: Synthesis of 6-(cyclopropanecarboxamido)-N-methyl-1-(methylamino)-2,7-naphthyridine-4-carboxamide

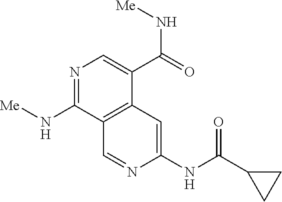 [SMILES: CNC(=O)c1cnc(NC)c2cnc(NC(=O)C3CC3)cc12]

To a stirring mixture of 6-cyclopropaneamido-1-(methylamino)-2,7-naphthyridine-4-carboxylic acid (26 mg; 0.09 mmol; 1.00 eq.) in DMF (1 mL) was added HAITI (51.8 mg; 0.136 mmol; 1.50 eq.). The reaction was stirred at room temperature for 20 m. To this reaction mixture was added DIPEA (35.1 mg; 0.272 mmol; 3.00 eq.) and MeNH₂·HCl (7.2 mg; 0.108 mmol; 1.2 eq.). The reaction was stirred at room temperature for 1 h. Upon completion, the resulting mixture was purified by flash chromatography on pre-packed C18 column using 20-80% of MeCN in water (10 mmol/L NH₄HCO₃) to provide 6-cyclopropaneamido-N-methyl-1-(methylamino)-2,7-naphthyridine-4-carboxamide as a white solid (1.4 mg, 9%), LCMS (ESI) m/z 300.1, [M+H]⁺. ¹H NMR (400 MHz, DMSO-d₆) δ 10.89 (s, 1H), 9.32 (s, 1H), 8.85 (s, 1H), 8.21 (s, 1H), 8.20-8.13 (m, 2H), 3.00 (d, J=4.4 Hz, 3H), 2.76 (d, J=4.4 Hz, 3H), 2.07-1.99 (m, 1H), 0.86-0.77 (m, 4H).

### Example 125: Synthesis of N-(5-ethynyl-8-(methylamino)-2,7-naphthyridin-3-yl)cyclopropanecarboxamide

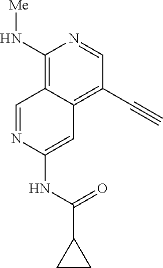 [SMILES: C#Cc1cnc(NC)c2cnc(NC(=O)C3CC3)cc12]

### Step 1: N-(8-(methylamino)-5-((trimethylsilyl)ethynyl)-2,7-naphthyridin-3-yl)cyclopropanecarboxamide

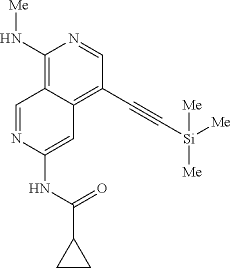 [SMILES: CNc1ncc(C#C[Si](C)(C)C)c2cc(NC(=O)C3CC3)ncc12]

To a stirring mixture of N-(5-bromo-8-(methylamino)-2,7-naphthyridin-3-yl)cyclopropanecarboxamide (500 mg; 1.557 mmol; 1.00 eq.) in DMF (7 mL, deoxygenated prior to use) was added Cut (9 mg; 0.047 mmol; 0.03 eq.), Pd(PPh₃)₂Cl₂ (21.9 mg; 0.031 mmol; 0.02 eq.), PPh₃ (8.2 mg; 0.031 mmol; 0.02 eq.), piperidine (205 mg; 2.411 mmol; 1.55 eq.) and ethynyltrimethylsilane (504.6 mg; 5.137 mmol; 3.30 eq.) at room temperature. The reaction was stirred at 90° C. for 16 h. The reaction mixture was monitored via LCMS. The reaction was diluted with EtOAc (100 mL), the organic phase was washed with a sat. NaCl solution (3×10 mL). The organic phase was dried with Na₂SO₄, filtered and the solvent was removed under reduced pressure. The residue was purified by flash chromatography on pre-packed C18 column using 20-60% of MeCN in water (10 mmol/L NH₄HCO₃) to provide N-(8-(methylamino)-5-((trimethylsilyl)ethynyl)-2,7-naphthyridin-3-yl)cyclopropanecarboxamide as a yellow solid (397 mg, 75%). LCMS (ESI) m/z 339.2 [M+H]⁺.

### Step 2: PV-(5-ethynyl-8-(methylamino)-2,7-naphthyridin-3-yl)cyclopropanecarboxamide

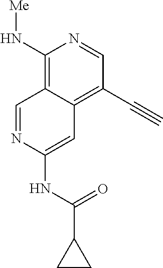 [SMILES: C#Cc1cnc(NC)c2cnc(NC(=O)C3CC3)cc12]

To a stirring mixture of N-(8-(methylamino)-5-((trimethylsilyl)ethynyl)-2,7-naphthyridin-3-yl)cyclopropanecarboxamide (397 mg; 1.17 mmol; 1.00 eq.) in MeOH (10 mL) was added K₂CO₃(431 mg; 3.16 mmol; 2.70 eq.) at room temperature under nitrogen atmosphere. The reaction was stirred at room temperature for 2 h. Upon completion, the reaction was diluted with EtOAc (100 mL), the organic phase was washed with saturated NaCl solution (2×10 mL). The organic phase was dried with Na₂SO₄, filtered and the solvent was removed under reduced pressure. The residue was purified by flash chromatography on silica gel column using 1-9% of MeOH in CH₂Cl₂ as eluent to provide N-(5-ethynyl-8-(methylamino)-2,7-naphthyridin-3-yl)cyclopropanecarboxamide as a yellow solid (300 mg, 96%). Then 10 mg of the crude product was purified by flash chromatography on pre-packed C18 column using 20-60% of MeCN in water (10 mmol/L NH₄HCO₃) to provide N-(5-ethynyl-8-(methylamino)-2,7-naphthyridin-3-yl)cyclopropanecarboxamide as a white solid (2.9 mg). LCMS (ESI) m/z 267.1, [M+H]⁺. ¹H NMR (400 MHz, DMSO-d₆) δ 11.06 (s, 1H), 9.34 (s, 1H), 8.52 (s, 1H), 8.31-8.24 (m, 1H), 8.21 (s, 1H), 4.35 (s, 1R), 2.99 (d J=4.4 Hz, 3H), 2.11-2.03 (m, 1H), 0.91-0.80 (m, 4H).

### Example 126: Synthesis of N-(5-(3-bromoisoxazol-5-yl)-8-(methylamino-2,7-naphthyridin-3-yl)cyclopropanecarboxamide

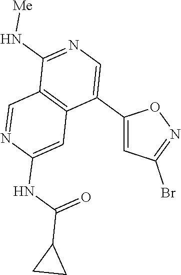 [SMILES: CNc1ncc(-c2cc(Br)no2)c2cc(NC(=O)C3CC3)ncc12]

To a stirring mixture of N-(5-ethynyl-8-(methylamino)-2,7-naphthyridin-3-yl)cyclopropanecarboxamide (80 mg; 0.300 mmol; 1.00 eq.) in EtOAc/water (5:1, 3.6 mL) was added KHCO₃(90.3 mg; 0.902 mmol; 3.00 eq.) and hydroxycarbonimidic dibromide (140.1 mg; 0.691 mmol; 2.30 eq.) at room temperature. The reaction was stirred at room temperature for 1 hour. Another portion of KHCO₃(90.3 mg; 0.902 mmol; 3.00 eq.) and hydroxycarbonimidic dibromide (140.1 mg; 0.691 mmol; 2.30 eq.) were added at room temperature. The reaction was stirred at room temperature for 1.5 h. The progress of the reaction was monitor via LCMS. The reaction was diluted with EtOAc (50 mL), the organic phase was washed with saturated NaCl solution (1×5 mL). The organic phase was dried with Na₂SO₄, filtered and the solvent was removed under reduced pressure. The residue was purified by flash chromatography on silica gel column using 1-9% of MeOH in CH₂Cl₂ as eluent to provide N-(5-(3-bromoisoxazol-5-yl)-8-(methylamino)-2,7-naphthyridin-3-yl)cyclopropanecarboxamide (70 mg, crude). Then 20 mg of the crude product was purified by reverse phase preparative HPLC (Prep-C18, 5 μM OBD column, 30×150 mm, waters; gradient elution of 40-48% MeCN in water over a 8 min period, where both water and MeCN contain 10 mmol/L NH₄HCO₃, flow rate: 60 mL/min, detector UV wavelength: 254 nm) to provide N-(5-(3-bromoisoxazol-5-yl)-8-(methylamino)-2,7-naphthyridin-3-yl)cyclopropanecarboxamide as a yellow solid (4.7 mg). LCMS (ESI) m/z 388.0, [M+H]⁺. ¹H NMR (400 MHz, DMSO-d₆) δ 11.12 (s, 1H), 9.42 (s, 1H), 8.72 (s, 1H), 8.60-8.54 (m, 1H), 8.46 (s, 1H), 7.03 (s, 1H), 3.05 (d, J=4.4 Hz, 3H), 2.12-2.02 (m, 1H), 0.90-0.82 (m, 4H).

### Example 127: Synthesis of N-(5-(3-cyclopropyl-3-oxoprop-1-yn-1-yl)-8-(methylamino)-2,7-naphthyridin-3-yl)cyclopropanecarboxamide

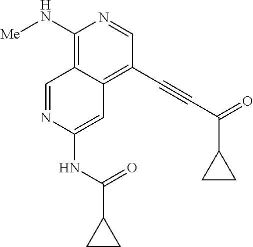 [SMILES: CNc1ncc(C#CC(=O)C2CC2)c2cc(NC(=O)C3CC3)ncc12]

### Step 1: 1-cyclopropyl-3-(trimethylsilyl)prop-2-yn-1-one

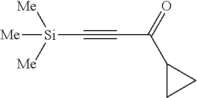 [SMILES: C[Si](C)(C)C#CC(=O)C1CC1]

To a stirring mixture of CuI (38.9 mg; 0.204 mmol; 0.04 eq.) and Pd(PPh₃)₂Cl₂ (71.6 mg; 0.102 mmol; 0.02 eq.) in Tiff (10 mL, deoxygenated prior to use) was added cyclopropanecarbonyl chloride (530.6 mg; 5.076 mmol; 1.00 eq.), Et₃N (519.9 mg; 5.138 mmol; 1.01 eq.) and ethynyltrimethylsilane (500 mg; 5.091 mmol; 1.00 eq.). The reaction was stirred at room temperature for 1 h under N₂ atmosphere. The desired product was observed via LCMS. The reaction was quenched with a saturated NH₄Cl solution (20 mL), then extracted with EtOAc (3×20 mL). The combined organic phase was washed with a saturated NaCl solution (1×5 mL). The solution was dried with Na₂SO₄, filtered and the solvent was removed under reduced pressure. The residue was purified by flash chromatography on silica gel column using 1-10% of EtOAc in petroleum ether as eluent to provide 1-cyclopropyl-3-(trimethylsilyl)prop-2-yn-1-one as a yellow oil (520 mg, 61%). LCMS (ESI) m/z 167.1, [M+H]⁺.

### Step 2: N-(5-(3-cyclopropyl-3-oxoprop-1-yn-1-yl)-8-(methylamino)-2,7-naphthyridin-3-yl)cyclopropanecarboxamide

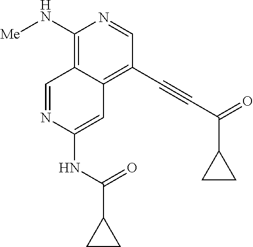 [SMILES: CNc1ncc(C#CC(=O)C2CC2)c2cc(NC(=O)C3CC3)ncc12]

A mixture of Pd(PPh₃)₂Cl₂ (45.6 mg; 0.062 mmol; 0.10 eq.), PPh₃, (32.7 mg; 0.124 mmol; 0.20 eq.), CuI (59.3 mg; 0.312 mmol; 0.50 eq.) and N-(5-bromo-8-(methylamino)-2,7-naphthyridin-3-yl)cyclopropanecarboxamide (200 mg; 0.625 mmol; 1.00 eq.) in DMF (10 mL, deoxygenated prior to use) was added Et₃N (145.1 mg; 1.43 mmol; 2.30 eq.) and 1-cyclopropyl-3-(trimethylsilyl)prop-2-yn-1-one (155.6 mg; 0.937 mmol; 1.50 eq.). To this mixture was added a solution of TBAF in THF (1 M) (0.93 mL; 0.93 mmol; 1.50 eq.). The reaction mixture was stirred at 90° C. for 3 hours. The resulting mixture was diluted with EtOAc (100 mL), the organic phase was washed with a saturated NaCl solution (3×10 mL). The organic phase was dried with Na₂SO₄, filtered and the solvent was removed under reduced pressure. The residue was purified by flash chromatography on silica gel column using 1-10% of MeOH in CH₂Cl₂ as eluent to provide the crude product. The crude product was purified by flash chromatography on pre-packed C18 column using 20-50% MeCN in water (10 mmol/L NH₄HCO₃) as eluent to provide N-(5-(3-cyclopropyl-3-oxoprop-1-yl)-1-yl)-8-(methylamino)-2,7-naphthyridin-3-yl)cyclopropanecarboxamide as a white solid (100 mg, 48%). Then 2.1 mg of the desired product was delivered. LCMS (ESI) m/z 335.1, [M+H]⁺. ¹H NMR (400 MHz, DMSO-d₆) δ 11.12 (5, 1H), 9.38 (s, 1H), 8.74-8.67 (m, 1H), 8.49 (s, 1H), 8.42 (s, 1H), 3.04 (d, J=4.4 Hz, 3H), 2.25-2.15 (m, 1H), 2.13-2.02 (m, 1H), 1.27-1.08 (m, 4H), 0.93-0.80 (m, 4H).

### Example 128: Synthesis of N-((methylamino)-5-propionyl-2,7-naphthyridin-3-yl)cyclopropanecarboxamide

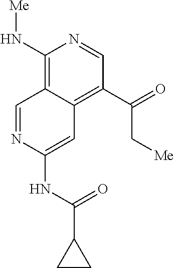 [SMILES: CCC(=O)c1cnc(NC)c2cnc(NC(=O)C3CC3)cc12]

### Step 1: (Z)—N-(5-(1-ethoxyprop-1-en-1-yl)-8-(methylamino)-2,7-naphthyridin-3-yl)cyclopropanecarboxamide

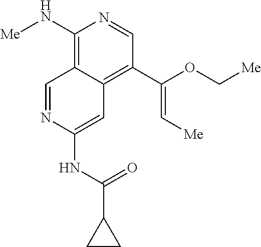 [SMILES: C/C=C(\OCC)c1cnc(NC)c2cnc(NC(=O)C3CC3)cc12]

To a stirring mixture of N-(5-bromo-8-(methylamino)-2,7-naphthyridin-3-yl)cyclopropanecarboxamide (50 mg; 0.156 mmol; 1.00 eq.) in ethylene glycol (2 mL, deoxygenated prior to use) was added Pd(OAc)₂ (0.7 mg; 0.003 mmol; 0.02 eq.), DPPP (2.6 mg; 0.006 mmol; 0.04 eq.), and Et₃N (31.6 mg; 0.312 mmol; 2.00 eq.). The reaction was stirred at 80° C. for 2 minutes, followed by the addition of (E)-1-ethoxyprop-1-ene (26.9 mg; 0.312 mmol; 2.00 eq.). The reaction was stirred at 100° C. for 1 h. The resulting mixture was diluted with EtOAc (50 mL), the organic phase was washed with a saturated NaCl solution (3×5 mL). The solution was dried with Na₂SO₄, filtered and the solvent was removed under reduced pressure. The residue was purified by flash chromatography on silica gel column using 1-10% of MeOH in CH₂Cl₂ as eluent to provide (Z)—N-(5-(1-ethoxyprop-1-en-1-yl)-8-(methylamino)-2,7-naphthyridin-3-yl)cyclopropanecarboxamide as a yellow solid (17 mg, 33%). LCMS (ESI) m/z 327.2, [M+H]⁺.

### Step 2: N-(5-(3-cyclopropyl-3-oxoprop-1-yn-1-yl)-8-(methylamino)-2,7-naphthyridin-3-yl)cyclopropanecarboxamide

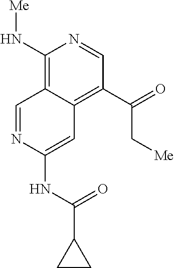 [SMILES: CCC(=O)c1cnc(NC)c2cnc(NC(=O)C3CC3)cc12]

To a stirring mixture of solution of (Z)—N-(5-(1-ethoxyprop-1-en-1-yl)-8-(methylamino)-2,7-naphthyridin-3-yl)cyclopropanecarboxamide (17 mg; 0.026 mmol; 1.00 eq.) in DMF (2 mL) was added a solution of HCl solution (0.26 mL, 5%) at 0° C. The reaction was stirred at room temperature for overnight. The desired product was observed via LCMS. The product mixture was purified by flash chromatography on pre-packed C18 column using 20-50% MeCN in water (10 mmol/L-NH₄HCO₃) as eluent to provide N-(5-(3-cyclopropyl-3-oxoprop-1-yn-1-yl)-8-(methyl amino)-2,7-naphthyridin-3-yl)cyclopropanecarboxamide as a yellow solid (1.9 mg, 24%). LCMS (ESI) m/z 299.1, [M+H]⁺. ¹H NMR (400 MHz, DMSO-d₆) δ 10.91 (s, 1H), 9.49 (s, 1H), 9.35 (s, 1H), 8.84 (s, 1H), 8.65-8.60 (m, 1H), 3.06 (d, J=4.4 Hz, 3H), 2.96 (q, J=7.2 Hz, 2H), 2.08-2.02 (m, 1H), 1.09 (t, J=7.2 Hz, 3H), 0.90-0.80 (m, 4H).

### Example 129: Synthesis of (E)-N-(8-(methylamino)-5-(prop-1-en-1-yl)-2,7-naphthyridin-3-yl)cyclopropanecarboxamide

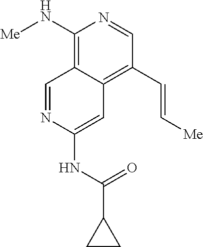 [SMILES: C/C=C/c1cnc(NC)c2cnc(NC(=O)C3CC3)cc12]

A mixture of N-(5-bromo-8-(methylamino)-2,7-naphthyridin-3-yl)cyclopropanecarboxamide (50 mg; 0.156 mmol; 1.00 eq.), Pd(OAc)₂ (0.7 mg; 0.003 mmol; 0.02 eq.) and DPPP (2.6 mg; 0.006 mmol; 0.04 eq.) in ethylene glycol (2 mL, deoxygenated prior to use). To this reaction mixture was added Et₃N (31.6 mg; 0.312 mmol; 2.00 eq.). The reaction was stirred at 80° C. for 2 minutes; followed by the addition of (E)-1-ethoxyprop-1-ene (26.9 mg; 0.312 mmol; 2.00 eq.). The reaction was stirred at 100° C. for 1 hours. The resulting mixture was diluted with EtOAc (50 mL), the organic phase was washed with a saturated NaCl solution (3×5 mL). The solution was dried with Na₂SO₄, filtered and the solvent was removed under reduced pressure. The residue was purified by flash chromatography on silica gel column using 110% of MeOH in CH₂Cl₂ as eluent to provide the crude product. Then the crude product was purified by reverse phase preparative HPLC (Prep-C18, 5 μM OBD column, 19×150 mm, waters; gradient elution of 22-32% MeCN in water over a 8 min period, where both water and MeCN contain 10 mmol/L NH₄HCO₃, flow rate: 60 mL/min, detector UV wavelength: 254 nm) to provide (L)-N-(8-(methylamino)-5-(prop-1-en-1-yl)-2,7-naphthyridin-3-yl)cyclopropanecarboxamide as a yellow solid (2.6 mg, 35%). LCMS (ESI) m/z 283.1, [M+H]⁺. ¹H NMR (400 MHz, DMSO-d₆) δ 11.00 (s, 1H), 9.32 (s, 1H), 8.43 (s, 1H), 8.08 (s, 1H), 7.92-7.85 (m, 1H), 6.57 (d, J=14.8 Hz, 1H), 6.13-6.03 (m, 1H), 2.96 (d, J=4.4 Hz, 3H), 2.10-2.02 (m, 1H), 1.89 (dd, J=6.4, 1.2 Hz, 3H), 0.90-0.79 (m, 4H).

### Example 130: Synthesis of N-(8-(methylamino)-5-(2-methyloxazol-5-yl)-2,7-naphthyridin-3-yl)cyclopropanecarboxamide

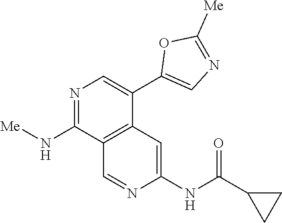 [SMILES: CNc1ncc(-c2cnc(C)o2)c2cc(NC(=O)C3CC3)ncc12]

To a stirring mixture of N-(5-ethynyl-8-(methyl amino)-2, 7-naphthyridin-3-yl)cyclopropanecarboxamide (50 mg; 0.188 mmol; 1.00 eq.) in MeCN (2.5 mL) was added (triphenyl((1,1,1-trifluoro-N-((trifluoromethyl)sulfonyl)methyl)sulfonamido)-phosphaneyl)gold (13.9 mg; 0.019 mmol; 0.10 eq.), 8-methylquinoline 1-oxide (38.9 mg; 0.244 mmol; 1.30 eq.) and methanesulfonic acid (20.0 mg; 0.208 mmol; 1.11 eq.) at room temperature. The reaction was stirred at 60° C. for 16 h. The desired product could be detected by LCMS. The resulting mixture was concentrated under vacuum. The residue was purified by flash chromatography on silica gel column using 1-9% of MeOH in CH₂Cl₂ as eluent to provide N-(8-(methyl amino)-5-(2-methyloxazol-5-yl)-2,7-naphthyridin-3-yl)cyclopropanecarboxamide (20 mg, crude). Then crude product was purified by reverse phase preparative HPLC (Prep-C18, 5 μM YMC-Actus Triart column, 30×150 mm, waters; gradient elution of 19-29% MeCN in water over a 10 min period, where both water and MeCN contain 10 mmol/L NH₄HCO₃, flow rate: 60 mL/min, detector UV wavelength: 254 nm) to provide N-(8-(methylamino)-5-(2-methyloxazol-5-yl)-2,7-naphthyridin-3-yl)cyclopropanecarboxamide as a yellow solid (2.1 mg, 3%), LCMS (ESI) m/z 324.1, [M+H]⁺. ¹H NMR (400 MHz, DMSO-d₆) δ 11.07 (s, 1H), 9.39 (s, 1H), 8.60 (s, 1H), 8.24 (s, 1H), 8.24-8.20 (m, 1H), 7.19 (s, 1H), 3.02 (d, J=4.4 Hz, 3H), 2.48 (s, 3H), 2.10-2.02 (m, 1H), 0.89-0.79 (m, 4H).

### Example 131: Synthesis of N-(5-(1-benzyl-1H-pyrazol-3-yl)-8-(methylamino)-2,7-naphthyridin-3-yl)cyclopropanecarboxamide

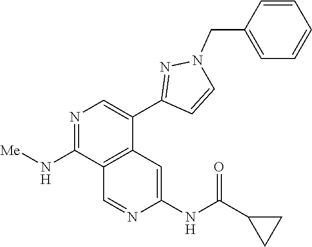 [SMILES: CNc1ncc(-c2ccn(Cc3ccccc3)n2)c2cc(NC(=O)C3CC3)ncc12]

### Step 1: (1-benzyl-1H-pyrazol-3-yl)boronic acid

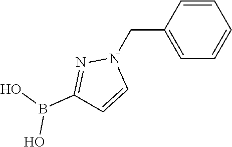 [SMILES: OB(O)c1ccn(Cc2ccccc2)n1]

To a stirring mixture of 3-(4,4,5,5-tetramethyl-1,3,2-dioxaborolan-2-yl)-1H-pyrazole (500 mg; 2.577 mmol; 1.00 eq.) in DMF (5 mL) was added NaH (206.1 mg; 5.154 mmol; 2.00 eq., 60%) at 0° C. and stirred at room temperature for 30 min under nitrogen atmosphere. To this reaction mixture was added (bromomethyl)benzene (528.8 mg; 3.092 mmol; 1.20 eq.) at room temperature. The resulting mixture was stirred for 1 h at room temperature. The desired product was observed via LCMS. The resulting mixture was quenched with a sat. NH₄Cl solution (0.2 mL). The resulting mixture was purified by flash chromatography on pre-packed C18 column using 10-40% of MeCN in water (10 mmol/L NH₄HCO₃) as eluent to provide (1-benzyl-1-pyrazol-3-yl)boronic acid as a off-white oil (180 mg, 24%). LCMS (ESI) m/z 203.1, [M+H]⁺.

### Step 2: N-(5-(1-benzyl-1H-pyrazol-3-yl)-8-(methylamino)-2,7-naphthyridin-3-yl)cyclopropanecarboxamide

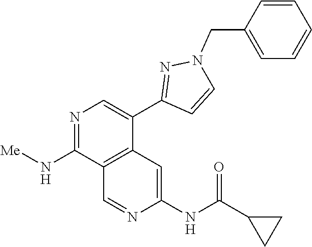 [SMILES: CNc1ncc(-c2ccn(Cc3ccccc3)n2)c2cc(NC(=O)C3CC3)ncc12]

To a stirring mixture of (1-(benzyl-1H-pyrazol-3-yl)boronic (31.4 mg; 0.156 mmol; 1.00 eq.) in a mixed solvent of 1,4-dioxane/water (5:1, 1.2 mL) was added XPhos Pd G₃ (13.2 mg; 0.016 mmol; 0.1 eq.), XPhos (7.4 mg; 0.016 mmol, 0.10 eq.), K₃PO₄ (99.1 mg; 0.468 mmol; 3.00 eq.) and N-(5-bromo-8-(methylamino)-2,7-naphthyridin-3-yl)cyclopropanecarboxamide (50 mg; 0.156 mmol; 1.00 eq.) at room temperature. The resulting mixture was stirred for 2 h at 90° C. under nitrogen atmosphere. Upon completion, the mixture was allowed to cool down to room temperature. The resulting mixture was concentrated under reduced pressure. The residue was purified by flash chromatography on silica gel column using 1-8% of MeOH in CH₂Cl₂ as eluent to provide 25 mg of the crude product. The crude product was purified by flash chromatography on pre-packed C18 column using 10-40% of MeCN in water (10 mmol/1. NH₄HCO₃) as eluent to provide N-(5-(1-benzyl-1H-pyrazol-3-yl)-8-(methylamino)-2, 7-naphthyridin-3-yl)cyclopropanecarboxamide as a off-white solid (21.0 mg, 33%). LCMS (ESI) m/z 399.2, [M+H]⁺. ¹H NMR (400 MHz, DMSO-d₆) 10.88 (s, 1H), 9.35 (s, 1H), 9.00 (s, 1H), 8.21 (s, 14), 7.96-7.92 (m, 1H), 7.90 (d, J=2.0 Hz, 1H), 7.45-7.28 (m, 5H), 6.53 (d, J=2.0 Hz, 1H), 5.37 (s, 2H), 3.00 (d, J=4.4 Hz, 31-1), 2.08-2.02 (m, 1H), 0.90-0.80 (m, 4H).

### Example 132: Synthesis of N-(8-(methylamino)-5-(4-((1-methylpiperidin-4-yl)oxy)phenyl)-2, 7-naphthyridin-3-yl)cyclopropanecarboxamide

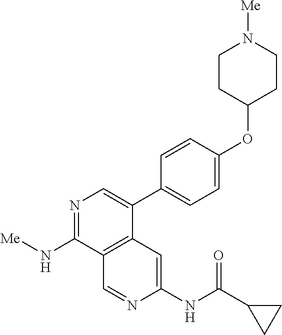 [SMILES: CNc1ncc(-c2ccc(OC3CCN(C)CC3)cc2)c2cc(NC(=O)C3CC3)ncc12]

### Step 1: 1-methyl-4-(4-4,4,5,5-tetramethyl-1,3,2-dioxaborolan-2-yl)phenoxy)piperidine

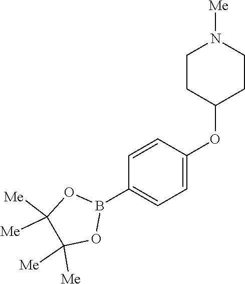 [SMILES: CN1CCC(Oc2ccc(B3OC(C)(C)C(C)(C)O3)cc2)CC1]

To a stirring mixture of 4-(4,4,5,5-tetramethyl-1,3,2-dioxaborolan-2-yl)phenol (100 mg; 0.454 mmol; 1.00 eq.) in THF (2 mL) was added PPh₃ (238.3 mg; 0.908 mmol; 2.00 eq.), 1-methylpiperidin-4-ol (104.6 mg; 09308 mmol; 2.00 eq.), and DIAD (183.7 mg; 0.908 mmol; 2.00 eq.). The reaction mixture was stirred overnight at room temperature under nitrogen atmosphere. The reaction was monitored by LCMS. Upon completion, the resulting mixture was concentrated under reduced pressure. The crude product was purified by flash chromatography on pre-packed C18 column using 20-50% MeCN in water (10 mmol/L NH₄HCO₃) to provide 1-methyl-4-(4-(4,4,5,5-tetramethyl-1,3,2-dioxaborolan-2-yl)phenoxy)piperidine as a white solid (48 mg; 34%). LCMS (ESI) m/z 318.2 [M+H]⁺.

### Step 2: N-(8-(methylamino)-5-(4-((1-methylpiperidin-4-yl)oxy)phenyl)-2,7-naphthyridin-3-yl)cyclopropanecarboxamide

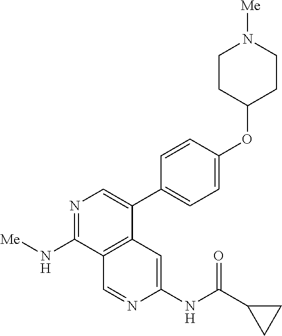 [SMILES: CNc1ncc(-c2ccc(OC3CCN(C)CC3)cc2)c2cc(NC(=O)C3CC3)ncc12]

A mixture of N-(5-bromo-8-(methylamino)-2,7-naphthyridin-3-yl)cyclopropanecarboxamide (36 mg; 0.112 mmol; 1.00 eq.), 1-methyl-4-(4-(4,4,5,5-tetramethyl-1,3,2-dioxaborolan-2-yl)phenoxy)piperidine (36 mg; 0.112 mmol; 1.00 eq.), Pd(DtBPF)Cl₂ (8 mg; 0.011 mmol; 0.10 eq.) and K₃PO₄(48 mg; 0.224 mmol; 2.00 eq.) in 1,4-dioxane/water (5:1, 1.2 mL) was stirred for 2 h at 90° C. under nitrogen atmosphere. The reaction was monitored by LCMS. The reaction mixture was concentrated under reduced pressure. The residue was purified by flash chromatography on silica gel column using 2-10% of MeOH in CH₂Cl₂ as eluent to afford the crude product. The crude product was re-purified by flash chromatography on pre-packed C18 column using 20-50% MeCN in water (10 mmol/L NH₄HCO₃) to provide N-(8-(methylamino)-5-(4-((1-methylpiperidin-4-yl)oxy)phenyl)-2,7-naphthyridin-3-yl)cyclopropanecarboxamide as a yellow solid (12.4 mg, 26%), LCMS (ESI) m/z 432.2, [M+H]⁺. ¹H NMR (400 MHz, Methanol-d₄) δ 9.25 (s, 1H), 8.39 (s, 1H), 7.83 (s, 1H), 7.44-7.36 (m, 2H), 7.19-7.11 (m, 2H), 4.83-4.79 (m, 1H), 3.52-3.39 (m, 4H), 3.12 (s, 3H), 2.93 (s, 3H), 2.31-2.11 (m, 4H), 1.99-1.90 (m, 1H), 0.99-0.86 (m, 4H)

### Example 133: Synthesis of N-(8-(methylamino)-5-(5-methyloxazol-2-yl)-2,7-naphthyridin-3-yl)cyclopropanecarboxamide

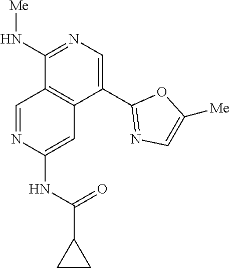 [SMILES: CNc1ncc(-c2ncc(C)o2)c2cc(NC(=O)C3CC3)ncc12]

A mixture of N-(5-bromo-8-(methylamino)-2,7-naphthyridin-3-yl)cyclopropanecarboxamide (60 mg; 0.187 mmol; 1.00 eq.), 5-methyl-2-(tributylstannyl)oxazole (104.3 mg; 0.280 mmol; 1.50 eq.), Pd(PPh₃)₄ (43.3 mg; 0.037 mmol; 0.20 eq.), CuI (7.12 mg; 0.037 mmol; 0.2 eq.) and LiCl (15.8 mg; 0.373 mmol; 2.00 eq.) in 1,4-dioxane (3 mL) was stirred for 3 h at 100° C. Upon completion, the resulting mixture was concentrated under reduced pressure. The residue was purified by flash chromatography on silica gel column using MeOH/CH₂Cl₂ (0-20%) as eluent to provide N-(8-(methylamino)-5-(5-methyloxazol-2-yl)-2,7-naphthyridin-3-yl)cyclopropanecarboxamide as a yellow solid (36.4 mg, crude). The crude product was purified by flash chromatography on pre-packed C18 column using 20-60% of MeCN in water (10 mmol/L NH₄HCO₃) to provide N-(8-(methyl amino)-5-(5-methyloxazol-2-yl)-2,7-naphthyridin-3-yl)cyclopropanecarboxamide as a yellow solid (5.7 mg, 9%). LCMS (ESI) m/z 324.1, [M+H]⁺. ¹H NMR (400 MHz, DMSO-d₆) δ 10.96 (s, 1H), 9.51 (s, 1H), 9.39 (s, 1H), 8.60 (s, 1H), 8.41-8.35 (m, 1H), 7.00 (s, 1H), 3.04 (d, J=4.4 Hz, 3H), 2.38 (s, 3H), 2.09-2.04 (m, 1H), 0.90-0.81 (m, 4H).

### Example 134: Synthesis of N-(8-(methylamino)-5-(phenylamino)-2,7-naphthyridin-3-yl)cyclopropanecarboxamide

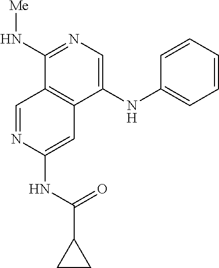 [SMILES: CNc1ncc(Nc2ccccc2)c2cc(NC(=O)C3CC3)ncc12]

A mixture of N-(5-bromo-8-(methylamino)-2,7-naphthyridin-3-yl)cyclopropanecarboxamide (40 mg; 0.125 mmol; 1.00 eq.), aniline (11.6 mg; 0.125 mmol; 1.00 eq.), Ethos Pd G₄ (11.5 mg; 0.013 mmol; 0.10 eq.), Ethos (7 mg; 0.013 mmol; 0.10 eq.) and Cs₂CO₃(81.2 mg; 0.250 mmol; 2.00 eq.) in 1,4-dioxane (1.5 mL) was stirred for 12 h at 100° C. under nitrogen atmosphere. The reaction was monitored by LCMS. The reaction was concentrated and the residue was purified by flash chromatography on silica gel column using MeOH/CH₂Cl₂ (2-10%) as eluent to afford the crude product. The crude product was purified by flash chromatography on pre-packed C18 column using 20-50% MeCN in water (10 mmol/L NH₄HCO₃) to provide N-(8-(methylamino)-5-(phenylamino)-2,7-naphthyridin-3-yl)cyclopropanecarboxamide as a yellow solid (28.4 mg, 69%). LCMS (ESI) m/z 334.2, [M+H]⁺. ¹⁻H NMR (400 MHz, DMSO-d₆) δ 10.90 (s, 1H), 9.34 (s, 1H), 8.26 (s, 1H), 7.88 (s, 1H), 7.84-7.81 (m, 1H), 7.42 (s, 1H), 7.07-7.01 (m, 2H), 6.60-6.49 (m, 3H), 2.98 (d, J=4.4 Hz, 3H), 2.09-1.99 (m, 1H), 0.82-0.71 (m, 4H).

### Example 135 and Example 136: Synthesis of (E)-N-(5-(4-methoxystyryl)-8-(methylamino)-2, 7-naphthyridin-3-yl)cyclopropanecarboxamide and (Z)—N-(5-(4-methoxystyryl)-8-(methylamino)-2,7-naphthyridin-3-yl)cyclopropanecarboxamide

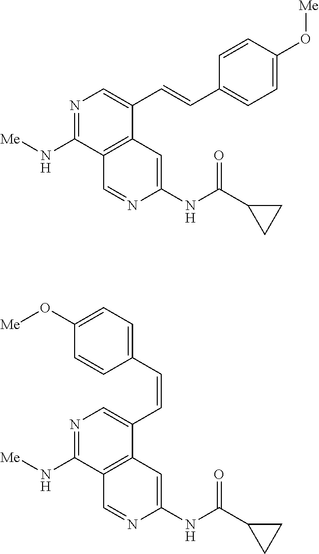 [SMILES: CNc1ncc(/C=C/c2ccc(OC)cc2)c2cc(NC(=O)C3CC3)ncc12 ; CNc1ncc(/C=C\c2ccc(OC)cc2)c2cc(NC(=O)C3CC3)ncc12]

To a stirring mixture of N-(5-bromo-8-(methylamino)-2,7-naphthyridin-3-yl)cyclopropanecarboxamide (50.0 mg; 0.156 mmol; 1.00 eq.) in DMF (2 mL) was added Pd(PPh₃)₂Cl₂ (10.9 mg; 0.016 mmol; 0.10 eq.), —Na₂CO₃ (330.0 mg; 0.312 mmol; 2.00 eq.) and 1-methoxy-4-vinylbenzene (20.8 mg; 0.156 mmol; 1.00 eq.) at room temperature. The resulting mixture was stirred for 2 h at 100° C. under nitrogen atmosphere. The desired product was observed via. LCMS. The mixture was cooled to room temperature. The resulting mixture was concentrated under reduced pressure. The residue was purified by flash chromatography on silica gel column using 1-6% of MeOH in CH₂Cl₂ as eluent to provide 15 mg mixture of the crude product. The mixture crude product was purified by reverse phase preparative HPLC (Prep-C18, XBridge Shield RP18 OBD column, 30×150 mm, waters; gradient elution of 35-45% MeCN in water over a 10 min period, where both water and MeCN contain 10 mmol/L NH₄HCO₃, flow rate: 60 mL/min, detector UV wavelength: 254 nm) to provide (E)-N-(5-(4-methoxystyryl)-8-(methylamino)-2,7-naphthyridin-3-yl)cyclopropanecarboxamide as a yellow solid (8.9 mg, 15%). LCMS (ESI) m/z 375.2, [M+H]⁺. ¹H NMR. (400 MHz, DMSO-d₆) δ 11.02 (s, 1H), 9.35 (s, 1H), 8.56 (s, 1H), 8.30 (s, 1H), 8.03-7.95 (m, 1H), 7.55-7.47 (m, 2H), 7.22 (d, J=16.4 Hz, 1H), 7.04-6.95 (m, 3H), 3.79 (s, 3H), 3.01 (d, J=4.4 Hz, 3H), 2.13-2.02 (m, 1H), 0.92-0.79 (m, 4H); And (4)-N-(5-(4-methoxystyryl)-8-(methylamino)-2,7-naphthyridin-3-yl)cyclopropanecarboxamide as a white solid (2.7 mg, 4%). LCMS (ESI) 375.2, [M+H]⁺. ¹H NMR (400 MHz, DMSO-d₆) δ 10.81 (s, 1H), 9.33 (s, 1H), 7.99 (s, 1H), 7.95-7.92 (m, 1H), 7.84 (s, 1H), 7.26-7.20 (m, 2H), 6.89-6.81 (m, 2H), 5.78 (d, J=1.2 Hz, 1H), 5.16 (d, J=1.2 Hz, 1H), 3.73 (s, 3H), 3.00 (d, J=4.4 Hz, 3H), 2.00-1.91 (m, 1H), 0.79-0.72 (m, 4H).

### Example 137: Synthesis of N-(8-(methylamino)-5-(phenylethynyl)-2,7-naphthyridin-3-yl)cyclopropanecarboxamide

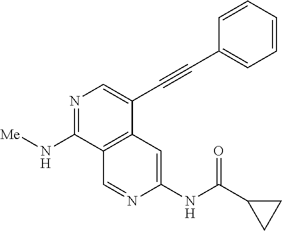 [SMILES: CNc1ncc(C#Cc2ccccc2)c2cc(NC(=O)C3CC3)ncc12]

A mixture of N-(5-bromo-8-(methylamino)-2,7-naphthyridin-3-yl)cyclopropanecarboxamide (100 mg; 0.311 mmol; 1.00 eq.), CuI (1.8 mg; 0.009 mmol; 0.02 eq.), Pd(PPh₃), Cl₂ (4.4 mg; 0.006 mmol; 0.02 eq.), piperidine (26.5 mg; 0.311 mmol; 1.00 eq.) and PPh₃ (1.6 mg; 0.006 mmol; 0.02 eq.) in DMF (3 mL) was stirred for 12 h at 90° C. under nitrogen atmosphere. The reaction was monitored via LCMS. The resulting mixture was concentrated under reduced pressure. The residue was purified by flash chromatography on silica gel column using 2-10% of MeOH in CH₂Cl₂ as eluent to afford the crude desired product. The product was re-purified by flash chromatography on pre-packed C18 column using 20-50% MeCN in water (10 mmol/L, NH₄HCO₃) to provide N-(8-(methylamino)-5-(phenylethynyl)-2,7-naphthyridin-3-yl)cyclopropanecarboxamide as a white solid (20.7 mg, 19.4%). LCMS (ESI) m/z 343.1, [M+H]⁺. ¹⁻H NMR (400 MHz, DMSO-d₆) δ 11.06 (s, 1H), 9.37 (s, 1H), 8.66 (s, 1H), 8.36-8.30 (m, 1H), 8.28 (s, 1H), 7.61-7.36 (m, 5H), 3.02 (d, J=4.4 Hz, 3H), 2.11-2.02 (m, 1H), 0.95-0.80 (m, 4H).

### Example 138: Synthesis of N-(8-(methylamino)-5-(3-methylisothiazol-5-yl)-2,7-naphthyridin-3-yl)cyclopropanecarboxamide

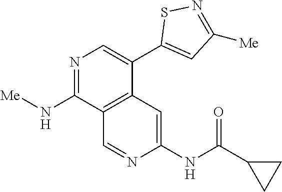 [SMILES: CNc1ncc(-c2cc(C)ns2)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (50.0 mg; 0.136 mmol; 1.00 eq.), 5-bromo-3-methylisothiazole (24.1 mg; 0.135 mmol; LOU eq.), XPhos Pd G₃ (11.5 mg; 0.014 mmol; 0.10 eq.), XPhos (6.5 mg; 0.014 mmol; 0.10 eq.) and K₃PO₄ (86.4 mg; 0.407 mmol; 3.00 eq.) in a mixture solvent of 1,4-dioxane/water (7:1, 2.4 mL) was stirred for 1 h at 90° C. under nitrogen atmosphere. The desired product was observed via LCMS. The resulting mixture was concentrated under reduced pressure. The residue was purified by flash chromatography on silica gel column using MeOH/CH₂Cl₂ (0-20%) as the eluent to provide a brown crude solid (30.1 mg). The crude product was purified by C18 column using 33-62% of MeCN in water (10 mmol/L NH₄HCO₃) as eluent to provide N-(8-(methylamino)-5-(3-methylisothiazol-5-yl)-2,7-naphthyridin-3-yl)cyclopropanecarboxamide as a white solid (14.9 mg, 32%). LCMS (ESI) m/z 340.1, [M+H]⁺. ¹⁻H NMR (400 MHz, DMSO-d₆) δ 11.07 (s, 1H), 9.40 (s, 1H), 8.62 (s, 1H), 8.30-8.27 (m, 1H), 8.19 (s, 1H), 7.34 (s, 1H), 3.02 (d, J=4.4 Hz, 3H), 2.48 (s, 3R), 2.09-2.04 (m, 1H), 0.90-0.78 (m, 4H).

### Examples 139-145: Each compound in Table 6 below was prepared following the same experimental procedure as described previously in Example 138

TABLE 6

| Example | Structure and Name | MS and ¹H NMR |
|---|---|---|
| Example 139 | 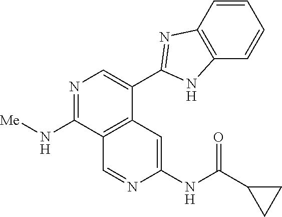 [SMILES: CNc1ncc(-c2nc3ccccc3[nH]2)c2cc(NC(=O)C3CC3)ncc12] | LCMS (ESI) m/z 359.2, [M + H]⁺. ¹H NMR (400 MHz, DMSO-d₆) δ 12.60 (s, 1H), 10.94 (s, 1H), 9.41 (s, 1H), 9.35 (s, 1H), 8.49 (s, 1H), 8.34-8.26 (m, 1H), 7.65 (d, J = 7.5 Hz, 1H), 7.55-7.48 (m, 1H), 7.26-7.15 (m, 2H), 3.06 (d, J = 4.4 Hz, 3H), 2.11-2.01 (m, 1H), 0.87-0.77 (m, 4H). |
| | N-(5-(1H-benzo[d]imidazol-2- | |
| | yl)-8-(methylamino)-2,7- | |
| | naphthyridin-3- | |
| | yl)cyclopropanecarboxamide | |
| Example 140 | 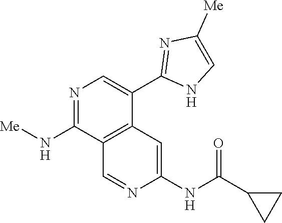 [SMILES: CNc1ncc(-c2nc(C)c[nH]2)c2cc(NC(=O)C3CC3)ncc12] | LCMS (ESI) m/z 323.2, [M + H]⁺. ¹H NMR (400 MHz, DMSO-d₆) δ 12.13 (s, 1H), 10.89 (s, 1H), 9.35 (s, 1H), 9.13 (s, 1H), 8.21 (s, 1H), 8.11-8.02 (m, 1H), 6.85 (s, 1H), 3.01 (d, J = 4.4 Hz, 3H), 2.22 (s, 3H), 2.07-2.03 (m, 1H), 0.82- 0.78 (m, 4H). |
| | N-(5-(4-methyl-1H-imidazol-2- | |
| | yl)-8-(methylamino)-2,7- | |
| | naphthyridin-3- | |
| | yl)cyclopropanecarboxamide | |
| Example 141 | 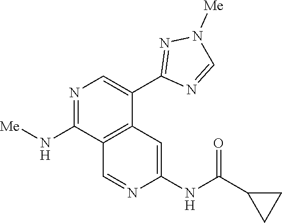 [SMILES: CNc1ncc(-c2ncn(C)n2)c2cc(NC(=O)C3CC3)ncc12] | LCMS (ESI) m/z 324.1, [M + H]⁺. ¹H NMR (400 MHz, DMSO-d₆) δ 10.95 (s, 1H), 9.40 (s, 1H), 9.36 (s, 1H), 8.56 (s, 1H), 8.53 (s, 1H), 8.44-8.25 (m, 1H), 3.94 (s, 3H), 3.04 (d, J = 4.4 Hz, 3H), 2.10-2.02 (m, 1H), 0.88-0.78 (m, 4H). |
| | N-(S-(1-methyl-1H-1,2,4- | |
| | triazol-3-yl)-8-(methylamino)- | |
| | 2,7-naphthyridin-3-yl) | |
| | cyclopropanecarboxamide | |
| Example 142 | 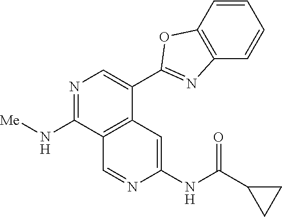 [SMILES: CNc1ncc(-c2nc3ccccc3o2)c2cc(NC(=O)C3CC3)ncc12] | LCMS (ESI) m/z 360.1, [M + H]⁺. ¹H NMR (400 MHz, DMSO-d₆) δ 11.04 (s, 1H), 9.68 (s, 1H), 9.44 (s, 1H), 8.92 (s, 1H), 8.71-8.60 (m, 1H), 7.80-7.70 (m, 2H), 7.49-7.36 (m, 2H), 3.09 (d, J = 3.2 Hz, 3H), 2.16-2.05 (m, 1H), 0.96-0.82 (m, 4H). |
| | N-(5-(benzo[d]oxazol-2-yl)-8- | |
| | (methylamino)-2,7- | |
| | naphthyridin-3- | |
| | yl)cyclopropanecarboxamide | |
| Example 143 | 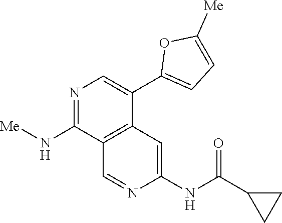 [SMILES: CNc1ncc(-c2ccc(C)o2)c2cc(NC(=O)C3CC3)ncc12] | LCMS (ESI) m/z 323.1, [M + H]⁺. ¹H NMR (400 MHz, DMSO-d₆) δ 11.01 (s, 1H), 9.37 (s, 1H), 8.75 (s, 1H), 8.22 (s, 1H), 8.15-8.04 (m, 1H), 6.50 (d, J = 3.2 Hz, 1H), 6.24 (d, J = 2.4 Hz, 1H), 3.01 (d, J = 4.4 Hz, 3H), 2.35 (s, 3H), 2.10- 2.04 (m, 1H), 0.92-0.82 (m, 4H). |
| | N-(8-(methylamino)-5-(5- | |
| | methylfuran-2-yl)-2,7- | |
| | naphthyridin-3-yl) | |
| | cyclopropanecarboxamide | |
| Example 144 | 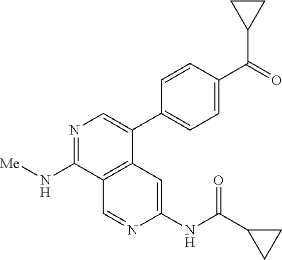 [SMILES: CNc1ncc(-c2ccc(C(=O)C3CC3)cc2)c2cc(NC(=O)C3CC3)ncc12] | LCMS (ESI) m/z 387.2, [M + H]⁺. ¹H NMR (400 MHz, DMSO-d₆) δ 10.99 (s, 1H), 9.41 (s, 1H), 8.40 (s, 1H), 8.16 (d, J = 8.0 Hz, 2H), 8.12-8.07 (m, 1H), 8.02 (s, 1H), 7.61 (d, J = 8.0 Hz, 2H), 3.03 (d, J = 4.4 Hz, 3H), 3.00-2.93 (m, 1H), 2.05-2.01 (m, 1H), 1.14-1.05 (m, 4H), 0.85-0.72 (m, 4H). |
| | N-(5-(4- | |
| | (cyclopropanecarbonyl)phenyl)- | |
| | 8-(methylamino)-2,7- | |
| | naphthyridin-3- | |
| | yl)cyclopropanecarboxamide | |
| Example 145 | 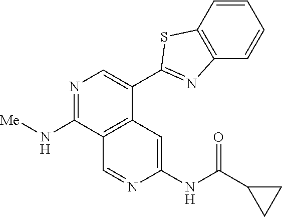 [SMILES: CNc1ncc(-c2nc3ccccc3s2)c2cc(NC(=O)C3CC3)ncc12] | LCMS (ESI) m/z 376.1, [M + H]⁺. ¹H NMR (400 MHz, DMSO-d₆) δ 11.03 (s, 1H), 9.59 (s, 1H), 9.44 (s, 1H), 8.62 (s, 1H), 8.54 (q, J = 4.4 Hz, 1H), 8.13 (d, J = 8.0 Hz, 1H), 8.00 (d, J = 8.0 Hz, 1H), 7.58-7.53 (m, 1H), 7.47-7.42 (m, 1H), 3.08 (d, J = 4.4 Hz, 3H), 2.11-2.04 (m, 1H), 0.95-0.81 (m, 4H). |
| | N-(5-(benzo[d]thiazol-2-yl)-8- | |
| | (methylamino)-2,7- | |
| | naphthyridin-3-yl) | |
| | cyclopropanecarboxamide | |

### Example 146: Synthesis of N-(8-(methylamino)-5-(3-phenylisothiazole-5-yl)-2,7-naphthyridin-3-yl)cyclopropanecarboxamide

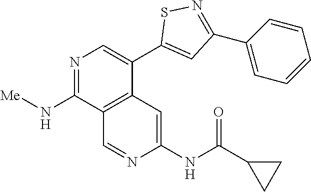 [SMILES: CNc1ncc(-c2cc(-c3ccccc3)ns2)c2cc(NC(=O)C3CC3)ncc12]

### Step 1: 5-chloro-3-phenylisothiazole

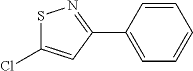 [SMILES: Clc1cc(-c2ccccc2)ns1]

To a stirring mixture of CuCl₂ (58 mg; 0.426 mmol; 1.50 eq.) and tert-butylnitrite (44 mg; 0.426 mmol, 1.50 eq.) in MeCN (1 mL). The reaction mixture was stirred for 2 min at 60° C. To this mixture was added a solution of 3-phenylisothiazol-5-amine (50 mg; 0.284 mmol; 1.00 eq.) in MeCN (0.5 mL) and the reaction mixture was stirred for 1 h at 60° C. The reaction was monitored by LCMS. The mixture was acidified to pH≈3 with a solution of HCl (1 N). The resulting mixture was extracted with CH₂Cl₂ (2×20 mL). The combined organic layers were washed with brine (1×5 mL), dried over anhydrous Na₂SO₄. After filtration, the filtrate was concentrated under reduced pressure. The residue was purified by flash chromatography on silica gel column using 5-15% of EtOAc in petroleum ether as eluent to afford 5-chloro-3-phenylisothiazole as a white solid (30 mg, 75%). LCMS (ESI) m/z 196.0, [M+H]⁺.

### Step 2: N-(8-(methylamino)-5-(3-phenylisothiazol-5-yl)-2,7-naphthyridin-3-yl)cyclopropane carboxamide

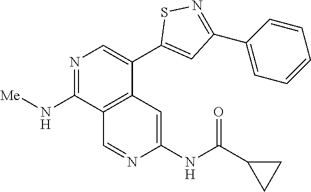 [SMILES: CNc1ncc(-c2cc(-c3ccccc3)ns2)c2cc(NC(=O)C3CC3)ncc12]

To a solution of N-(8-(methylamino)-5-(4,4,5,5-tetramethyl-1,3,2-dioxaborolan-2-yl)-2,7-naphthyridin-3-yl)cyclopropanecarboxamide (30 mg; 0.081 mmol; 1.00 eq.) in a mixture of 1,4-dioxane/water (5:1, 1.2 mL) was added 5-chloro-3-phenylisothiazole (16 mg; 0.081 mmol; 1.00 eq.), XPhos Pd G₃ (6.9 mg; 0.008 mmol; 0.10 eq.), XPhos (3.9 mg; 0.008 mmol; 0.10 eq.) and K₃PO₄ (34.6 mg; 0.162 mmol; 2.00 eq.). The resulting mixture was stirred for 3 h at 90° C. under nitrogen atmosphere. The reaction was monitored by LCMS. The residue was purified by flash chromatography on silica gel column using (MeOH/CH₂Cl₂: 2-10%) as eluent to afford N-(8-(methylamino)-5-(3-phenylisothiazol-5-yl)-2,7-naphthyridin-3-yl)cyclopropanecarboxamide (28.6 mg, crude). The crude product was re-purified by flash chromatography on pre-packed C18 column using 20-50% MeCN in water (10 mmol/L NH₄HCO₃) to provide N-(8-(methylamino)-5-(3-phenylisothiazol-5-yl)-2,7-naphthyridin-3-yl)cyclopropanecarboxamide as a white solid (8.6 mg, 27%). LCMS (ESI) m/z 402.1, [M+H]⁺. ¹H NMR (400 MHz, DMSO-d₆) δ 11.10 (s, 1H), 9.43 (s, 1H), 8.72 (s, 1H), 8.36-8.34 (m, 1H), 8.33 (s, 1H), 8.11 (s, 1H), 8.10-8.07 (m, 2H), 7.56-7.45 (m, 3H), 3.05 (d, J=4.4 Hz, 3H), 2.10-2.02 (m, 1H), 0.87-0.78 (m, 4H).

### Example 147: Synthesis of N-(5-(1-ethyl-1H-pyrazol-3-yl)-8-(methylamino)-2,7-naphthyridin-3-yl)cyclopropanecarboxamide

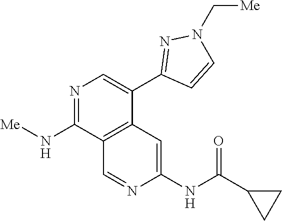 [SMILES: CCn1ccc(-c2cnc(NC)c3cnc(NC(=O)C4CC4)cc23)n1]

### Step 1: 3-bromo-1-ethyl-1H-pyrazole and 5-bromo-1-ethyl-1H-pyrazole

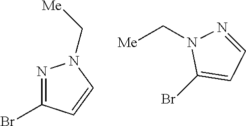 [SMILES: CCn1ccc(Br)n1 ; CCn1nccc1Br]

To a stirred solution of 3-bromo-1H-pyrazole (500.2 mg; 3.40 mmol, 1.00 eq.) and Cs₂CO₃ (2.21 g; 6.78 mmol; 2.00 eq.) in DMF (10 mL) was added iodoethane (796.6 mg; 5.10 mmol; 1.50 eq.). The resulting mixture was stirred for 2 h at room temperature. The desired product (two peaks with desired product mass) could be detected by LCMS. The resulting mixture was diluted with EtOAc (100 mL), A normal aqueous workup with EtOAc was followed. The organic combined organic layer was washed by brine (5 mL×3). The resulting EtOAc was dried over Na₂SO₄. After filtration, the filtrate was concentrated under reduced pressure. The residue was purified by flash chromatography on silica gel column using MeOH/CH₂Cl₂ (0-2%) to provide 3-bromo-1-ethyl-1H-pyrazole as a yellow oil (340.5 mg, 57%) and 5-bromo-1-ethyl-1H-pyrazole as a yellow oil (60 mg, 10%). LCMS (ESI) m/z 175.0, [M+H]⁺.

### Step 2: N-(5-(1-ethyl-1H-pyrazol-3-yl)-8-(methy(amino)-2,7-naphthyridin-3-yl) cyclopropanecarboxamide

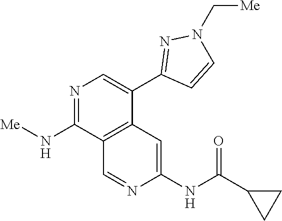 [SMILES: CCn1ccc(-c2cnc(NC)c3cnc(NC(=O)C4CC4)cc23)n1]

To a stirred solution of 3-bromo-1-ethyl-1H-pyrazole (36.8 mg; 0.21 mmol; 1.30 eq.) and N-(8-(methylamino)-5-(4,4,5,5-tetramethyl-1,3,2-dioxaborolan-2-yl)-2,7-naphthyridin-3-yl)cyclopropanecarboxamide (60 mg; 0.16 mmol; 1.00 eq.) in a mixture of 1,4-dioxane/water (10:1, 3.3 mL) were added XPhos Pd G₃ (13.7 mg; 0.016 mmol; 0.10 eq.), XPhos (7.7 mg; 0.016 mmol; 0.10 eq.), and K₃PO₄ (90 mg; 0.42 mmol; 2.00 eq.). The resulting mixture was stirred for 1 h at 90° C. under nitrogen atmosphere. The desired product was detected via LCMS. The resulting mixture was concentrated under reduced pressure. The residue was purified by flash chromatography on silica gel column using MeOH/CH₂Cl₂ (4-6%) to provide the crude product (20 mg). The crude product was further purified by flash chromatography on pre-packed C18 column using 50-70% MeCN/water (50-70% and 10 mmol/L NH₄HCO₃) to provide N-(5-(1-ethyl-1H-pyrazol-3-yl)-8-(methylamino)-2,7-naphthyridin-3-yl)cyclopropanecarboxamide as a off-white solid (10.2 mg, 30%). LCMS (ESI) m/z 337.2, [M+H]⁺. ¹H NMR. (400 MHz, DMSO-d₆) δ 10.87 (s, 1H), 9.35 (s, 1H), 9.01 (s, 1H), 8.20 (s, 1H), 7.96-7.91 (m, 1H), 7.81 (d, J=2.0 Hz, 1H), 6.47 (d, J=2.0 Hz, 1H), 4.19 (q, J=7.2 Hz, 2H), 3.00 (d, 4.4 Hz, 3H), 2.09-2.01 (m, 1H), 1.46 (t, J=7.2 Hz, 3H), 0.88-0.78 (m, 4H).

### Example 148: Synthesis of N-(5-(1-isopropyl-1H-pyrazol-3-yl)-8-(methylamino)-2,7-naphthyridin-3-yl)cyclopropanecarboxamide

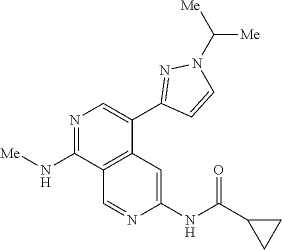 [SMILES: CNc1ncc(-c2ccn(C(C)C)n2)c2cc(NC(=O)C3CC3)ncc12]

### Step 1: 3-bromo-1-isopropyl-1H-pyrazole and 5-bromo-1-isopropyl-1H-pyrazole

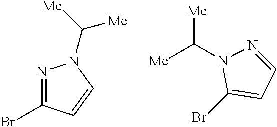 [SMILES: CC(C)n1ccc(Br)n1 ; CC(C)n1nccc1Br]

To a stirred solution of 3-bromo-1H-pyrazole (500.2 mg; 3.40 mmol; 1.00 eq.) and Cs₂CO₃ (2.21 g; 6.804 mmol; 2.00 eq.) in DMF (10 mL) was added 2-iodopropane (867.4 mg; 5.103 mmol; 1.50 eq.). The resulting mixture was stirred for 2 h at room temperature. The desired products (two peaks with desired product mass) were detected via LCMS. The resulting mixture was diluted with EtOAc (100 mL) and washed by brine (3×5 mL). The resulting mixture was concentrated under reduced pressure. The residue was purified by flash chromatography on silica gel column using MeOH/CH₂Cl₂ (1-2%) as eluent to provide 3-bromo-1-isopropylpyrazole as a yellow oil (370 mg, 57%) and 5-bromo-1-isopropyl-1H-pyrazole as a yellow oil (120 mg, 18%). LCMS (ESI) m/z 189.0, [M+H]⁺.

### Step 2: N-(5-(1-isopropyl-1H-pyrazol-3-yl)-8-(methylamino)-2,7-naphthyridin-3-yl)cyclopropanecarboxamide

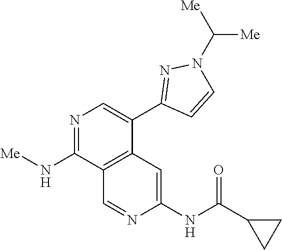 [SMILES: CNc1ncc(-c2ccn(C(C)C)n2)c2cc(NC(=O)C3CC3)ncc12]

To a stirred solution of 3-bromo-1-isopropylpyrazole (39.8 mg; 0.211 mmol; 1.30 eq.) and N-(8-(methylamino)-5-(4,4,5,5-tetramethyl-1,3,2-dioxaborolan-2-yl)-2,7-naphthyridin-3-yl)cyclopropanecarboxamide (60 mg; 0.16 mmol; 1.00 eq.) in a mixture solvent of 1,4-dioxane/water (10:1, 3.3 mL) were added XPhos Pd G₃ (13.7 mg; 0.016 mmol; 0.10 eq.), XPhos (7.7 mg; 0.016 mmol; 0.10 eq.) and K₃PO₄ (90 mg; 0.42 mmol; 2.00 eq.). The resulting mixture was stirred for 1 h at 90° C. under nitrogen atmosphere. The desired product was observed via LCMS. The resulting mixture was concentrated under reduced pressure. The residue was purified by flash chromatography on silica gel column using MeOH/CH₂Cl₂ (0-5%) as eluent to provide the title compound (20 mg, crude). This crude product was purified by flash chromatography on pre-packed C18 column using 20-60% MeCN in water (10 mmol/L NH₄HCO₃) to provide N-(5-(1-isopropyl-1H-pyrazol-3-yl)-8-(methylamino)-2,7-naphthyridin-3-yl)cyclopropanecarboxamide as a off-white solid (7.1 mg, 20%). LCMS (ESI) m/z 351.2, [M+H]⁺. ¹H NMR (400 MHz, DMSO-d₆) δ 10.87 (s, 1H), 9.35 (s, 1H), 9.09 (s, 1H), 8.21 (s, 1H), 7.98-7.92 (m, 1H), 7.83 (d, J=2.0 Hz, 1H), 6.47 (d, J=2.0 Hz, 1H), 4.60-4.48 (m, 1H), 3.00 (d, J=4.4 Hz, 3H), 2.09-2.01 (m, 1H), 1.50 (d, 6.8 Hz, 6H), 0.90-0.78 (m, 4H)

### Example 149: Synthesis of N-(5-(1-isobutyl-1H-pyrazol-3-yl)-8-(methylamino)-2,7-naphthyridin-3-yl)cyclopropanecarboxamide

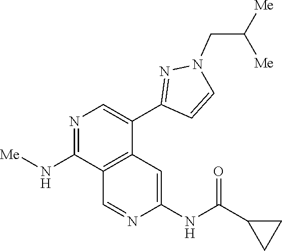 [SMILES: CNc1ncc(-c2ccn(CC(C)C)n2)c2cc(NC(=O)C3CC3)ncc12]

### Step 1: 3-bromo-1-isobutyl-1H-pyrazole and 5-bromo-1-isobutyl-1H-pyrazole

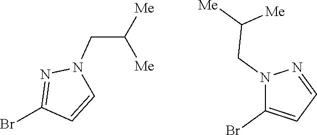 [SMILES: CC(C)Cn1ccc(Br)n1 ; CC(C)Cn1nccc1Br]

To a stirred solution of 3-bromo-1H-pyrazole (500.2 mg; 3.40 mmol; 1.00 eq.) and Cs₂CO₃ (2.21 g; 6.80 mmol; 2.00 eq.) in DMF (10 mL) was added 1-iodo-2-methylpropane (945.2 mg; 5.10 mmol; 1.5 eq.). The resulting mixture was stirred for 2 h at room temperature. The desired products (two peaks with desired product mass) were detected by LCMS. The resulting mixture was diluted with EtOAc (100 mL) and washed by brine (3×5 mL). The organic layer was concentrated under reduced pressure. The residue was purified by flash chromatography on a silica gel column using CH₂Cl₂ in petroleum ether (0-60%) as eluent to provide 3-bromo-1-isobutyl-1H-pyrazole as a yellow oil (150 mg, 21%) and 5-bromo-1-isobutyl-1H-pyrazole as a yellow oil (20 mg, 2%). LCMS (ESI) m/z 203.0, [M+H]⁺.

### Step 2: N-(5-(1-isobutyl-1H-pyrazol-3-yl)-8-(methylamino)-2,7-naphthyridin-3-yl)cyclopropanecarboxamide

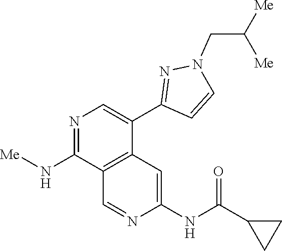 [SMILES: CNc1ncc(-c2ccn(CC(C)C)n2)c2cc(NC(=O)C3CC3)ncc12]

To a stirred solution of 3-bromo-1-isobutyl-1H-pyrazole (42.8 mg; 0.211 mmol; 1.30 eq.) and N-(8-(methylamino)-5-(4,4,5,5-tetramethyl-1,3,2-dioxaborolan-2-yl)-2,7-naphthyridin-3-yl)cyclopropanecarboxamide (60 mg; 0.16 mmol; 1.0 eq.) in a mixture solvent of 1,4-dioxane/water (10:1, 3.3 mL) were added XPhos Pd G₃ (13.7 mg; 0.016 mmol; 0.10 eq.), XPhos (7.7 mg; 0.016 mmol; 0.10 eq.) and K₃PO₄(90 mg; 0.42 mmol; 2.00 eq.). The resulting mixture was stirred for 1 h at 90° C. under nitrogen atmosphere. The desired product could be detected by LCMS. The resulting mixture was concentrated under reduced pressure. The residue was purified via a silica gel column using MeOH/CH₂Cl₂(0-5%) as eluent to provide the title compound (12 mg, crude). The crude product was purified by flash chromatography on pre-packed C18 column using 10-37% MeCN in water (10 mmol/L NH₄HCO₃) as eluent to provide N-(5-(1-isobutyl-1H-pyrazol-3-yl)-8-(methylamino)-2,7-naphthyridin-3-yl)cyclopropanecarboxamide as a off-white solid (6.9 mg, 23%). LCMS (ESI) m/z 365.2, [M+H]⁺. ¹H NMR (400 MHz, DMSO-d₆) δ 10.87 (s, 1H), 9.35 (s, 1H), 8.94 (s, 1H), 8.18 (s, 1H), 7.98-7.90 (m, 1H), 7.79 (d, J=2.0 Hz, 1H), 6.46 (d, J=2.0 Hz, 1H), 3.95 (d, J===7.2 Hz, 2H), 3.00 (d, J=4.4 Hz, 3H), 2.30-2.20 (m, 1H), 2.08-2.00 (m, 1H), 0.89 (d, J=6.8 Hz, 6H), 0.84-0.77 (m, 4H).

### Example 150: Synthesis of N-(5-(1-((1-methyl-1H-1,2,4-triazol-3-yl)methyl)-1H-pyrazol-3-yl)-8-(methylamino)-2,7-naphthyridin-3-yl)cyclopropanecarboxamide

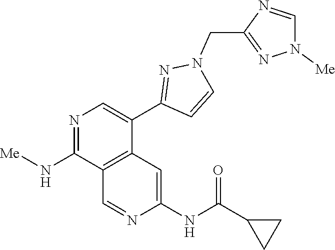 [SMILES: CNc1ncc(-c2ccn(Cc3ncn(C)n3)n2)c2cc(NC(=O)C3CC3)ncc12]

### Step 1: 3-((3-bromo-1H-pyrazol-1-yl)methyl)-1-methyl-1H-1,2,4-triazole

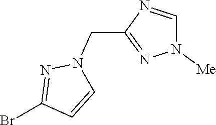 [SMILES: Cn1cnc(Cn2ccc(Br)n2)n1]

To a stirred solution of 3-promo-1H-pyrazole (212.2 mg; 1.442 mmol; 1.20 eq.) and Cs₂CO₃ (780.3 mg; 2.394 mmol; 2.00 eq.) in DMF (4 mL) was added 3-(chloromethyl)-1-methyl-1H-1,2,4-triazole hydrochloride (200.3 mg; 1.198 mmol; 1.00 eq.). The resulting mixture was stirred for 1 h at room temperature under nitrogen atmosphere. The desired product could be detected by LCMS. The resulting mixture was diluted with EtOAc (100 mL). The resulting mixture was washed with brine (3×5 mL). The organic layers were dried over anhydrous-Na₂SO₄. After filtration, the filtrate was concentrated under reduced pressure to provide 3-((3-bromo-1H-pyrazol-1-yl)methyl)-1-methyl-1H-1,2,4-triazole as a yellow oil (220 mg, 76%). LCMS (ESI) m/z 242.0, [M+H]⁺.

### Step 2: N-(5-(1-((1-methyl-1H-1,2,4-triazol-3-yl)methyl)-1H-pyrazol-3-yl)-8-(methylamino)-2,7-naphthyridin-3-yl)cyclopropanecarboxamide

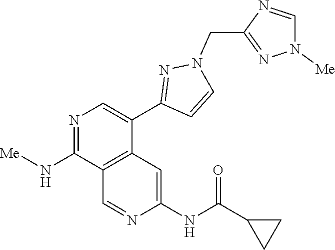 [SMILES: CNc1ncc(-c2ccn(Cc3ncn(C)n3)n2)c2cc(NC(=O)C3CC3)ncc12]

To a stirring solution of 3-((3-promo-1H-pyrazol-1-yl)methyl)-1-methyl-1H-1,2,4-triazole (85.2 mg; 0.352 mmol; 1.30 eq.) and N-(8-(methylamino)-5-(4,4,5,5-tetramethyl-1,3,2-dioxaborolan-2-yl)-2,7-naphthyridin-3-yl)cyclopropanecarboxamide (100.2 mg; 0.272 mmol; 1.00 eq.) in a mixture solvent of 1,4-dioxane/water (5:1, 2.4 mL) was added XPhos Pd G₃ (23.4 mg, 0.027 mmol; 0.10 eq.), XPhos (13.2 mg; 0.027 mmol; 0.10 eq.), K₃PO₄ (172.8 mg; 0.815 mmol; 3.00 eq.). The resulting mixture was stirred for 1 h at 90° C. under nitrogen atmosphere. The desired product was observed via LCMS. The resulting mixture was concentrated under reduced pressure. The residue was purified by flash chromatography on silica gel column using MeOH/CH₂Cl₂(5-20%) of as eluent to provide the crude product. The crude product was purified by reverse phase preparative HPLC (Prep-C18, XBridge Shield RP18 OBD column, 30×150 mm, waters; gradient elution of 15-25% MeCN in water over a 10 min period, where both water and MeCN contain 10 mmol/L NH₄HCO₃, flow rate: 10 mL/min, detector UV wavelength: 254 nm) to provide N-(5-(1-((1-methyl-1H-1,2,4-triazol-3-yl)methyl)-1H-pyrazol-3-yl)-8-(methylamino)-2,7-naphthyridin-3-yl)cyclopropanecarboxamide as a yellow solid (2.4 mg, 2%). LCMS (ESI) m/z 404.2, [M+H]⁺. ¹H NMR (400 MHz, DMSO-d₆) δ 10.88 (s, 1H), 9.35 (s, 1H), 8.84 (s, 1H), 8.43 (s, 1H), 8.17 (s, 1H), 8.00-7.92 (m, 1H), 7.85 (d, J=2.0 Hz, 1H), 6.50 (d, J=2.0 Hz, 1H), 5.38 (s, 2H), 3.85 (s, 3H), 3.00 (d, J=4.4 Hz, 3H), 2.10-2.00 (m, 1H), 0.90-0.78 (m, 4H).

### Example 151: Synthesis of N-(8-(methylamino)-5-(1-(thiazol-2-ylmethyl-1H-pyrazol-3-yl)-2, 7-naphthyridin-3-yl)cyclopropanecarboxamide

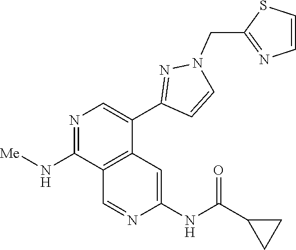 [SMILES: CNc1ncc(-c2ccn(Cc3nccs3)n2)c2cc(NC(=O)C3CC3)ncc12]

### Step 1: 2-((3-bromo-1H-pyrazol-1-yl)methyl)thiazole

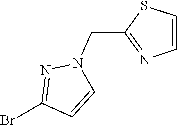 [SMILES: Brc1ccn(Cc2nccs2)n1]

A mixture of 3-bromo-1R-pyrazole (395.2 mg; 2,689 mmol; 1.20 eq.) in DMF (5 mL) was treated with NaH (107.8 mg; 4.492 mmol; 2.00 eq.) at 0° C. and stirred for 0.5 h at room temperature under nitrogen atmosphere. To this mixture was added 2-(chloromethyl)thiazole (300.0 mg; 2.246 mmol; 1.00 eq.) dropwise at 0° C. The resulting mixture was stirred for an additional of 2 h at room temperature. The desired product could be detected by LCMS. The reaction was quenched with a sat. NH₄Cl solution (50 mL) at 0° C. The resulting mixture was extracted with EtOAc (50 mL×3). The organic layers were washed with brine (10 mL) and dried over anhydrous Na₂SO₄. After filtration; the filtrate was concentrated under reduced pressure. The residue was purified via a silica gel column using 10-50% of CH₂Cl₂ in petroleum ether as eluent to provide 2-((3-bromo-1H-pyrazol-1-yl)methyl)thiazole as a yellow solid (309.0 mg, 56%). LCMS (ESI) m/z 243.9, [M+H]⁺.

### Step 2: N-(8-(methylamino)-5-(1-(thiazol-2-ylmethyl)-1H-pyrazol-3-yl)-2,7-naphthyridin-3-yl)cyclopropanecarboxamide

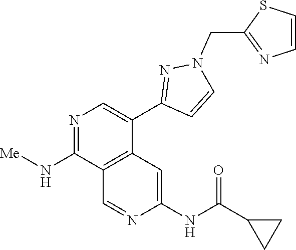 [SMILES: CNc1ncc(-c2ccn(Cc3nccs3)n2)c2cc(NC(=O)C3CC3)ncc12]

A mixture of 2((3-bromo-1H-pyrazol-1-yl)methyl)thiazole (66.2 mg; 0.271 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (100.0 mg; 0.272 mmol; 1.00 eq.) in a mixture solvent of 1,4-dioxane/water (5:1, 2.4 mL) was added XPhos Pd G₃ (22.9 mg; 0.027 mmol; 0.10 eq.) and XPhos (12.9 mg; 0.027 mmol; 0.10 eq.) and K₃PO₄. (172.9 mg; 0.815 mmol; 3.00 eq.). The resulting mixture was stirred 1 h at 90° C. under nitrogen atmosphere. The desired product could be detected by LCMS. The resulting mixture was concentrated under reduced pressure. The residue was purified by flash chromatography on silica gel column using 2-10% of MeOH in CH₂Cl₂ as eluent to provide the crude product. The crude product was purified by flash chromatography on pre-packed C18 column using 20-60% of MeCN in water (10 mmol/L NH₄HCO₃) to provide N-(8-(methylamino)-5-(1-(thiazol-2-ylmethyl)-1H-pyrazol-3-yl)-2,7-naphthyridin-3-yl)cyclopropanecarboxamide as a white solid (4.5 mg, 4%). LCMS (ESI) m/z 406.1, [M+H]⁺. ¹H NMR (400 MHz, DMSO-d₆) δ 10.90 (s, 1H), 9.36 (s, 1H), 8.85 (s, 1H), 8.21 (s, 1H), 8.02 (d, J=2.0 Hz, 1H), 8.01-7.97 (m, 1H), 7.79 (d, J=3.2 Hz, 1H), 7.69 (d, J=3.2 Hz, 1H), 6.57 (d, J=2.0 Hz, 1H), 5.75 (s, 2H), 3.10 (d, J=4.4 Hz, 3H), 2.09-2.01 (m, 1H), 0.88-0.76 (m, 4H).

### Example 152: Synthesis of N-(5-(1-((5-methyl-1,2,4-oxadiazol-3-yl)methyl)-4-pyrazol-3-yl)-8-(methylamino)-2,7-naphthyridin-3-yl)cyclopropanecarboxamide

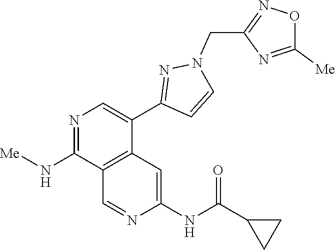 [SMILES: CNc1ncc(-c2ccn(Cc3noc(C)n3)n2)c2cc(NC(=O)C3CC3)ncc12]

### Step 1: 3-((3-bromo-1H-pyrazol-1-yl)methyl)-5-methyl-1,2,4-oxadiazole

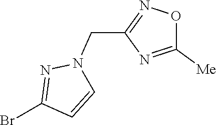 [SMILES: Cc1nc(Cn2ccc(Br)n2)no1]

A mixture of 3-bronco-1H-pyrazole (399 mg; 2.72 mmol; 1.20 eq.) in DMF (5.0 mL) was added Cs₂CO₃ (1.84 g; 5.66 mmol; 2.50 eq.) at room temperature followed by the addition of 3-(chloromethyl)-5-methyl-1,2,4-oxadiazole (300 mg; 2.26 mmol; 1.00 eq.) in portions at room temperature. The resulting mixture was stirred for 1 h at room temperature under nitrogen atmosphere. The desired product could be detected by LCMS. The mixture was allowed to cool down to room temperature. The resulting mixture was concentrated under reduced pressure. The residue was purified by flash chromatography on silica gel column using 25-60% of EtOAc in CH₂Cl₂ as eluent to provide 3((3-bromo-1H-pyrazol-1-yl)methyl)-5-methyl-1,2,4-oxadiazole as a off-white oil (150 mg, 27%). LCMS (ESI) m/z 243.0, [M+H]⁺.

### Step 2: N-(5-(1-((5-methyl-1,2,4-oxadiazol-3-yl)methyl)-1H-pyrazol-3-yl)-8-(methylamino-2,7-naphthyridin-3-yl)cyclopropanecarboxamide

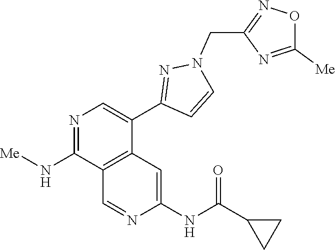 [SMILES: CNc1ncc(-c2ccn(Cc3noc(C)n3)n2)c2cc(NC(=O)C3CC3)ncc12]

A mixture of 3-((3-bromo-1H-pyrazol-1-yl)methyl)-5-methyl-1,2,4-oxadiazole (66.0 mg; 0.272 mmol; 1.00 eq.), N-(8-(methylamino)-5-(4,4,5,5-tetramethyl-1,3,2-dioxaborolan-2-yl)-2,7-naphthyridin-3-yl)cyclopropanecarboxamide) (100 mg; 0.272 mmol; 1.00 eq.) in a mixture solvent of 1,4-dioxane/water (5:1, 3 mL) was added Pd(DtBPF)Cl₂ (17.8 mg; 0.027 mmol; 0.100 eq.) and K₃PO₄ (115 mg; 0.542 mmol; 2.00 eq). The resulting mixture was stirred for 2 h at 90° C. under nitrogen atmosphere. The desired product was observed by LCMS. The residue was purified by flash chromatography on silica gel column using MeOH/CH₂Cl₂ (0-20%) as eluent to afford a yellow crude product (33 mg). The crude product was purified by C18 column using 25-60% of MeCN in water (10 mmol/L NH₄HCO₃) as eluent to provide N-(5-(1-((5-methyl-1,2,4-oxadiazol-3-yl)methyl)-1H-pyrazol-3-yl)-8-(methylamino)-2,7-naphthyridin-3-yl)cyclopropanecarboxamide as a white solid (14.6 mg, 13%). LCMS (ESI) m/z 405.2, [M+H]⁺. ¹H NMR (400 MHz, DMSO-d₆) δ 10.96 (s, 1H), 9.40 (s, 1H), 8.85 (s, 1H), 8.10 (s, 1H), 7.99 (d, J=2.0 Hz, 1H), 6.58 (d, J=2.0 Hz, 1H), 5.54 (s, 2H), 3.03 (d, J=4.4 Hz, 3H), 2.57 (s, 3H), 2.09-2.01 (m, 1H), 0.90-0.79 (m, 4H).

### Example 153A: Synthesis of N-(8-(methylamino)-5-(1-((tetrahydro-2H-thiopyran-4-yl)methyl)-1H-pyrazol-3-yl)-2,7-naphthyridin-3-yl)cyclopropanecarboxamide

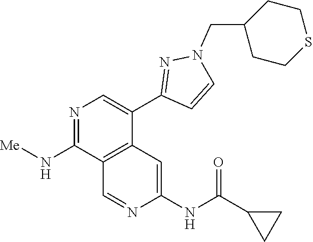 [SMILES: CNc1ncc(-c2ccn(CC3CCSCC3)n2)c2cc(NC(=O)C3CC3)ncc12]

### Step 1: (tetrahydro-2H-thiopyran-4-yl)methyl methanesulfonate

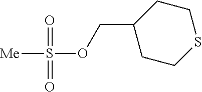 [SMILES: CS(=O)(=O)OCC1CCSCC1]

To a stirred solution of (tetrahydro-2H-thiopyran-4-yl)methanol (300.2 mg; 2.269 mmol; 1.00 eq.) and DIPEA (703.8 mg; 5.446 mmol; 2.4 eq.) in CH₂Cl₂ (4 mL) was added methanesulfonic anhydride (830.1 mg; 4.992 mmol; 2.20 eq.) in portions at 0° C. The resulting mixture was stirred for 1 h at room temperature. The residue was diluted with CH₇Cl₂ (80 mL) and washed with water mL). The organic layer was dried over anhydrous Na₂SO₄. After filtration, the filtrate was concentrated under reduced pressure to provide (tetrahydro-2H-thiopyran-4-yl)methyl methanesulfonate as a yellow solid (450 mg, 94%), LCMS (ESI) m/z 211.0, [M+H]⁺.

### Step 2: 3-bromo-1-((tetrahydro-2H-thiopyran-4-yl)methyl)-1H-pyrazole

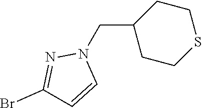 [SMILES: Brc1ccn(CC2CCSCC2)n1]

To a stirred solution of 3-bromo-1H-pyrazole (365.3 mg; 2,483 mmol; 1.49 eq.) and Cs₂CO₃ (1.08 g; 3.333 mmol; 2.00 eq.) in DMF (5 mL) was added (tetrahydro-2H-thiopyran-4-yl)methyl methanesulfonate (350.2 mg; 1.664 mmol; 1.00 eq.). The resulting mixture was stirred overnight at 50° C. under nitrogen atmosphere. The desired product could be detected by LCMS. The resulting mixture was diluted with CH₂Cl₂(70 mL). The resulting mixture was washed with a sat. NaCl solution (2×5 ML). The organic layers were dried over anhydrous Na₂SO₄. After filtration the filtrate was concentrated under reduced pressure. The residue was purified by flash chromatography on pre-packed C18 column using 55-60% of MeCN in water (10 mmol/L NH₄HCO₃) to provide 3-bromo-1-((tetrahydro-2H-thiopyran-4-yl)methyl)-1H-pyrazole as a white solid (190.2 mg, 43%), LCMS (ESI) m/z 261.0, [M+H]⁺.

### Step 3: N-(8-(methylamino)-5-(1-((tetrahydro-2H-thiopyran-4-yl)methyl)-1H-pyrazol-3-yl)-2,7-naphthyridin-3-yl)cyclopropanecarboxamide

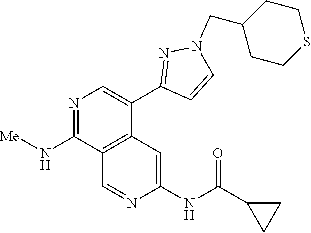 [SMILES: CNc1ncc(-c2ccn(CC3CCSCC3)n2)c2cc(NC(=O)C3CC3)ncc12]

To a stirred solution of 3-bromo-1-((tetrahydro-2H-thiopyran-4-yl)methyl)-1H-pyrazole (46.2 mg; 0.176 mmol; 1.30 eq.) and N-(8-(methylamino)-5-(4,4,5,5-tetramethyl-1,3,2-dioxaborolan-2-yl)-2,7-naphthyridin-3-yl)cyclopropanecarboxamide (50.2 mg; 0.136 mmol; 1.00 eq.) in a mixture solvent of 1,4-dioxane/water (5:1, 2.4 mL) was added XPhos Pd G₃ (11.5 mg; 0.014 mmol; 0.10 eq.), XPhos (6.5 mg; 0.014 mmol; 0.10 eq.), and K₃PO₄(86.2 mg; 0.405 mmol; 2.98 eq.). The resulting mixture was stirred for 1 h at 90° C. under nitrogen atmosphere. The desired product could be detected by LCMS. The resulting mixture was concentrated under reduced pressure. The residue was purified by flash chromatography on silica gel column using MeOH/CH₂Cl₂ (0-7%) as eluent to provide the crude product. The crude product was further purified by flash chromatography on pre-packed C18 column using 20-50% MeCN in water (0.05% formic acid) as eluent to provide N-(8-(methylamino)-5-(1-((tetrahydro-2H-thiopyran-4-yl)methyl)-1H-pyrazol-3-yl)-2,7-naphthyridin-3-yl)cyclopropanecarboxamide (5.2 mg, 9%). LCMS (ESI) m/z 423.2, [M+H]⁺. ¹H NMR (400 MHz, DMSO-d₆) δ 10.98 (s, 1H), 9.40 (s, 1H), 9.14 (s, 1H), 8.10 (s, 1H), 7.79 (d, 2.0 Hz, 1H), 6.48 (d, J=2.0 Hz, 1H), 4.00 (d, J=7.2 Hz, 2H), 3.04 (d, J=4.4 Hz, 3H), 2.75-2.65 (m, 2H), 2.59-2.52 (m, 2H), 2.30-2.19 (m, 1H), 2.09-2.02 (m, 1H), 1.85-1.77 (m, 2H), 1.36-1.26 (m, 2H), 0.89-0.79 (m, 4H).

### Example 153B: Synthesis of N-(5-(1-((1,1-dioxidotetrahydro-2H-thiopyran-4-yl)methyl)-1H-pyrazol-3-yl)-8-(methylamino)-2,7-naphthyridin-3-yl)cyclopropanecarboxamide

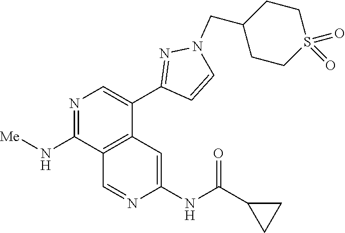 [SMILES: CNc1ncc(-c2ccn(CC3CCS(=O)(=O)CC3)n2)c2cc(NC(=O)C3CC3)ncc12]

### Step 1: 4((3-bromo-1H-pyrazol-1-yl)methyl)tetrahydro-2H-thiopyran 1,1-dioxide

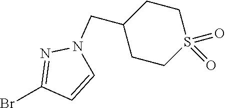 [SMILES: O=S1(=O)CCC(Cn2ccc(Br)n2)CC1]

A mixture of 3-bromo-1-((tetrahydro-2H-thiopyran-4-yl)methyl)-1H-pyrazole (50.2 mg; 0.191 mmol; 1.00 eq.) and m-CPBA (100.2 mg; 0.581 mmol; 3.00 eq.) in dichloromethane (2 mL) was stirred for 1 h at room temperature under air atmosphere. After completion of reaction, the mixture was diluted with CH₂Cl₂ (80 mL) and washed by water (2×10 mL). The organic layers were dried over anhydrous Na₂SO₄. After filtration, the filtrate was concentrated under reduced pressure. The residue was purified by flash chromatography on silica gel column using 0-5% of MeOH in CH₂Cl₂ as eluent to provide 4-((3-bromo-1H-pyrazol-1-yl)methyl)tetrahydro-2H-thiopyran 1,1-dioxide as a white solid (50.3 mg, 89%), LCMS (ESI) m/z 293.0, [M+H]⁺.

### Step 2: N-(5-(1-((1,1-dioxidotetrahydro-2H-thiopyran-4H)methyl)-1H-pyrazol-3-yl)-8-(methylamino)-2,7-naphthyridin-3-yl)cyclopropanecarboxamide

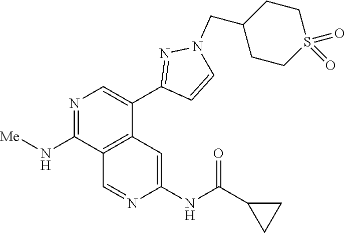 [SMILES: CNc1ncc(-c2ccn(CC3CCS(=O)(=O)CC3)n2)c2cc(NC(=O)C3CC3)ncc12]

To a stirred solution of 4-((3-bromo-1H-pyrazol-1-yl)methyl)tetrahydro-2H-thiopyran 1,1-dioxide (40.2 mg; 0.137 mmol, 1.00 eq.) and N-(8-(methylamino)-5-(4,4,5,5-tetramethyl-1,3,2-dioxaborolan-2-yl)-2,7-naphthyridin-3-yl)cyclopropanecarboxamide (50.1 mg; 0.136 mmol; 0.99 eq.) in a mixture solvent of 1,4-dioxane/water (5:1, 2.4 mL) was added XPhos Pd G₃ (11.5 mg; 0.014 mmol; 0.10 eq.), XPhos (6.5 mg; 0.014 mmol; 0.10 eq.) and K₃PO₄ (86.5 mg; 0.408 mmol; 2.97 eq.). The resulting mixture was stirred for 2 h at 90° C. under nitrogen atmosphere. The desired product could be detected by LCMS. The resulting mixture was concentrated under reduced pressure. The residue was purified by flash chromatography on silica gel column using MeOH/CH₂Cl₂ (5-8%) as eluent to provide crude product. The crude product was further purified by flash chromatography on pre-packed C18 column using 20-50% MeCN in water (10 mmol/L NH₄HCO₃) to afford N-(5-(1-((1,1-dioxidotetrahydro-2H-thiopyran-4-yl)methyl)-1H-pyrazol-3-yl)-8-(methylamino)-2,7-naphthyridin-3-yl)cyclopropanecarboxamide as a white solid (7.2 mg, 11%). LCMS (ESI) m/z 455.2, [M+H]⁺. NMR (400 MHz, DMSO-d₆) δ 10.93 (s, 1H), 9.35 (s, 1H), 9.17 (s, 1H), 8.21 (s, 1H), 7.98-7.95 (m, 1H), 7.79 (d, J=2.0 Hz, 1H), 6.47 (d, J=2.0 Hz, 1H), 4.09 (d, J=7.2 Hz, 2H), 3.21-3.02 (m, 4H), 3.00 (d, J=4.4 Hz, 3H), 2.63-2.56 (m, 1H), 2.10-2.03 (m, 1H), 1.87-1.80 (m, 2H), 1.73-1.62 (m, 21-f), 0.89-0.79 (m, 4H).

### Example 154: Synthesis of N-(8-(methylamino)-5-(1-(pyridin-2-ylmethyl)-1H-pyrazol-3-yl)-2,7-naphthyridin-3-yl)cyclopropanecarboxamide

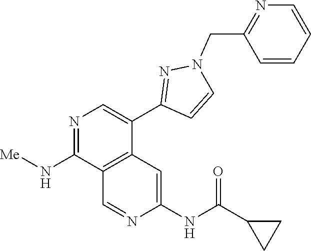 [SMILES: CNc1ncc(-c2ccn(Cc3ccccn3)n2)c2cc(NC(=O)C3CC3)ncc12]

### Step 1: 2-((3-bromo-1-pyrazol-1-yl)methyl)pyridine

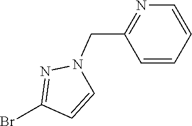 [SMILES: Brc1ccn(Cc2ccccn2)n1]

A mixture of 3-bromo-1/1-pyrazole (500 mg; 3.40 mmol; 1.00 eq.), 2-(bromomethyl)pyridine hydrobromide (1.29 g; 5.10 mmol; 1.50 eq.), and Cs₂CO₃ (30.33 g; 10.2 mmol; 3.00 eq.) in DMF (10.0 mL) was stirred for 3 h at room temperature under nitrogen atmosphere. The desired product was Observed via LCMS. The residue was dissolved in EtOAc (200 mL), washed with brine (3×20 mL). The organic solution was dried over anhydrous Na₂SO₄ and then concentrated under reduced pressure. The residue was purified by flash chromatography on silica gel column using 30-60% of EtOAc in petroleum ether as eluent to provide 2-((3-bromo-1H-pyrazol-1-yl)methyl)pyridine as a yellow crude oil (600 mg, 73%). LCMS (ESI) m/z 238.0, [M+H]⁺.

### Step 2: N-(8-(methylamino)-5-(1-(pyridin-2-ylmethyl)-1H-pyrazol-3-yl)-2,7-naphthyridin-3-yl)cyclopropanecarboxamide

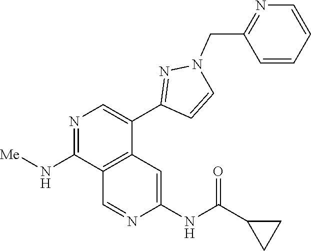 [SMILES: CNc1ncc(-c2ccn(Cc3ccccn3)n2)c2cc(NC(=O)C3CC3)ncc12]

To a stirring mixture of 2-((3-bromo-1H-pyrazol-1-yl)methyl)pyridine (25.9 mg; 0.109 mmol; 1.00 eq.), N-(8-(methylamino)-5-(4,4,5,5-tetramethyl-1,3,2-dioxaborolan-2-yl)-2,7-naphthyridin-3-yl)cyclopropanecarboxamide (40.0 mg; 0.109 mmol; 1.00 eq.) in 1,4-dioxane/water (5:1, 3 mL) was added XPhos Pd G₃ (9.19 mg; 0.011 mmol; 0.10 eq.), XPhos (5.18 mg; 0.011 mmol; 0.10 eq.) and K₃PO₄(69.2 mg; 0.327 mmol; 3.00 eq.) under nitrogen atmosphere. The mixture was stirred for 1 h at 90° C. The desired product was observed via LCMS. The resulting mixture was concentrated under reduced pressure. The residue was purified by flash chromatography on silica gel column using MeOH/CH₂Cl₂ (0-20%) as eluent to provide a yellow crude solid (20.0 mg). The crude product was further purified by reverse phase preparative HPLC (Prep-C18, 5 inn XBridge Prep Phenyl OBD Column, 19×150 mm, waters; gradient elution of MeCN/water (23-28%) over a 8 min period, where both water and MeCN contain 10 mmol/L NH₄HCO₃, flow rate: 60 mL/min, detector UV wavelength: 254 nm) to provide N-(8-(methylamino)-5-(1-(pyridin-2-ylmethyl)-1H-pyrazol-3-yl)-2,7-naphthyridin-3-yl)cyclopropanecarboxamide as a white solid (4.6 mg, 11%). LCMS (ESI) m/z 400.2, [M+H]⁺. ¹H NMR (400 MHz, DMSO-d₆) δ 10.91 (s, 1H), 9.35 (s, 1H), 8.93 (s, 1H), 8.56-8.54 (m, 1H), 8.20 (s, 1H), 7.99-7.97 (m, 1H), 7.96 (d, J=2.0 Hz, 1H), 7.80-7.75 (m, 1H), 7.34-7.30 (m, 1H), 7.27-7.24 (m, 1H), 6.56 (d, J=2.0 Hz, 1H), 5.49 (s, 2H), 3.00 (d, J=4.4 Hz, 3H), 2.07-2.01 (m, 1H), 0.87-0.78 (m, 4H).

### Example 155: Synthesis of N-(8-((2-hydroxyethyl)amino)-5-phenyl-2,7-naphthyridin-3-yl)cyclopropane carboxamide

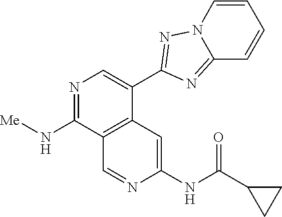 [SMILES: CNc1ncc(-c2nc3ccccn3n2)c2cc(NC(=O)C3CC3)ncc12]

### Step 1: 2-bromo-[1,2,4]triazolo[1,5-a]pyridine

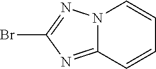

Toa mixture of tert-butyl nitrite (340 mg; 3.297 mmol; 1.47 eq.) in MeCN (12 mL) was added CuBr (480 mg; 3.346 mmol; 1.50 eq.). The reaction mixture was stirred at 60° C. for 10 min. To this mixture was added a solution of [1,2,4]triazolo[1,5-a]pyridin-2-amine (300 mg; 2.236 mmol; 1.00 eq.) in MeCN (5 mL) and stirred at 60° C. for 1 hour. The desired product was observed via LCMS. The reaction was filtered and washed with MeCN (3×10 mL). The filtrate was collected and concentrated under vacuum to afford 2-bromo-[1,2,4]triazolo[1,5-a]pyridine as a brown solid (300 mg, crude). This crude product was used in the next step without further purification. LCMS (ESI) m/z 198.0, [M+H]⁺.

### Step 2: N-(5-([1,2,4]triazolo[1,5-a]pyridin-2-yl)-8-(methylamino)-2,7-naphthyridin-3-yl)cyclopropanecarboxamide

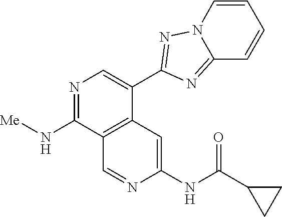 [SMILES: CNc1ncc(-c2nc3ccccn3n2)c2cc(NC(=O)C3CC3)ncc12]

To a mixture of XPhos Pd G₃ (92 mg; 0.109 mmol; 0.20 eq.), K₃PO₄ (350 mg; 1.649 mmol; 3.04 eq.) and XPhos (52 mg; 0.109 mmol; 0.20 eq.) in a mixture solvent of 1,4-dioxane/water (5:1, 6 mL) was added N-(8-(methylamino)-5-(4,4,5,5-tetramethyl-1,3,2-dioxaborolan-2-yl)-2,7-naphthyridin-3-yl)cyclopropanecarboxamide (200 mg; 0.543 mmol; 1 eq.) and 2-bromo-[1,2,4]triazolo[1,5-a]pyridine (107 mg; 0.540 mmol; 0.99 eq.) under nitrogen atmosphere. The reaction was stirred for 2 h at 60° C. The desired product was observed via LCMS. The reaction was concentrated under vacuum. The residue was purified by flash chromatography on silica gel column using 5-10% of MeOH in CH₂Cl₂ as eluent to afford the crude product. This crude product was purified by reverse phase preparative HPLC (Prep-C18, 5 μm XBridge Prep Phenyl OBD column, 19×150 mm, waters; gradient elution of 20-25% MeCN/water over a 8 min period, where both water and MeCN contain 10 mmol/L NH₄HCO₃, flow rate: 60 mL/mill, detector UV wavelength: 254 nm) to provide N-(5-([1,2,4]triazolo[1,5-a]pyridin-2-yl)-8-(methylamino)-2,7-naphthyridin-3-yl)cyclopropanecarboxamide as a off-white solid (12.5 mg, 6.4%). LCMS (ESI) m/z 360.1, [M+H]⁺. ¹H NMR (400 MHz, DMSO-d₆) δ 10.94 (s, 1H), 9.51 (s, 1H), 9.41 (s, 1H), 8.97-8.93 (m, 1H), 8.81 (s, 1H), 8.32-8.27 (m, 1H), 7.88-7.84 (m, 1H), 7.71-7.66 (m, 1H), 7.21-7.17 (m, 1H), 3.06 (d, J=4.4 Hz, 3H), 2.12-2.04 (m, 1H), 0.91-0.79 (m, 4H).

### Example 156: Synthesis of N-(5-(2-(azetidin-1-yl)-[1,2,4]triazolo[1,5-d]pyridin-7-yl)-8-(methylamino)-2,7-naphthyridin-3-yl)cyclopropanecarboxamide

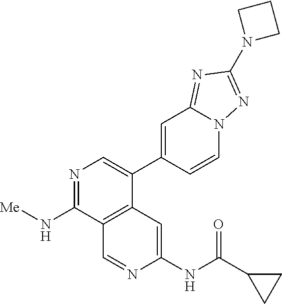 [SMILES: CNc1ncc(-c2ccn3nc(N4CCC4)nc3c2)c2cc(NC(=O)C3CC3)ncc12]

### Step 1: 2,7-dibromo-[1,2,4]triazolo[1,5-a]pyridine

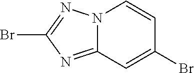 [SMILES: Brc1ccn2nc(Br)nc2c1]

To a solution of CuBr (504 mg; 3.51 mmol; 1.50 eq.) in MeCN (8.0 mL) was added t-BuONO (364 mg; 3.53 mmol; 1.50 eq.) with stirring. The mixture was stirred for 10 min at room temperature under nitrogen atmosphere. To this reaction mixture was added 7-bromo-[1,2,4]triazolo[1,5-a]pyridin-2-amine (500 mg; 2.35 mmol; 1.00 eq.) in MeCN (1 mL) was added in portions at room temperature. The reaction was stirred for 4 h at 80° C. The desired product could be detected by LCMS. The resulting mixture was diluted with EtOAc (200 mL), washed with brine (3×10 mL), dried over anhydrous Na₂SO₄. After filtration, the filtrate was concentrated under reduced pressure to provide 2,7-dibromo-[1,2,4]triazolo[1,5-a]pyridine as a green solid (194 mg, crude). LCMS (ESI) m/z 275.9, [M+H]⁺.

### Step 2: N-(5-(2-bromo-[1,2,4]triazolo[1,5-a]pyridin-7-yl)-8-(methylamino)-2,7-naphthyridin-3-yl)cyclopropanecarboxamide

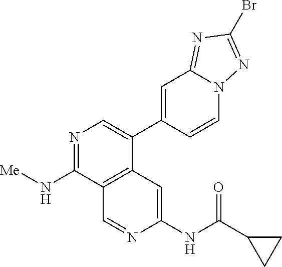 [SMILES: CNc1ncc(-c2ccn3nc(Br)nc3c2)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (194 mg; 0.527 mmol; 1.00 eq.) in a mixture solvent of 1.4-dioxane/water (5:1, 6 mL) was added 2,7-dibromo-[1,2,4]triazolo[1,5-a]pyridine (146 mg; 0.527 mmol; 1.00 eq.), Pd(DtBPF)Cl₂. (34.3 mg; 0.053 mmol; 0.10 eq.) and K₃PO₄ (22.3 mg; 1.05 mmol; 2.00 eq.). This reaction mixture was stirred for 1 h at room temperature under nitrogen atmosphere. The desired product was detected via LCMS. The resulting mixture was concentrated under reduced pressure. The residue was purified by flash chromatography on silica gel column using MeOH/CH₂Cl₂ (0-20%) as eluent to provide a yellow solid (118 mg, 51%). Then 10 mg of the crude product was purified by C18 column using 30-60% of MeCN in water (10 mmol/L NH₄HCO₃) as eluent to provide N-(5-(2-bromo-[1,2,4]triazolo[1,5-a]pyridin-7-yl)-8-(methylamino)-2,7-naphthyridin-3-yl)cyclopropanecarboxamide as a white solid (1.4 mg). LCMS (ESI) m/z 438.1, [M+H]⁺. ¹H NAM (400 MHz, DMSO-d₆) δ 11.02 (s, 1H), 9.42 (s, 1H), 9.06-9.02 (m, 1H), 8.39 (s, 1H), 8.23-8.18 (m, 1H), 8.12 (s, 1H), 7.86-7.84 (m, 7.36-7.33 (m, 1H), 3.04 (d, J=4.4 Hz, 3H), 2.06-1.98 (m, 1H), 0.82-0.76 (m, 4H).

### Step 3: N-(5-(2-(azetidin-1-yl)-[1,2,4]triazolo[1,5-a]pyridin-7-yl)-8-(methylamino)-2,7-naphthyridin-3-yl)cyclopropanecarboxamide

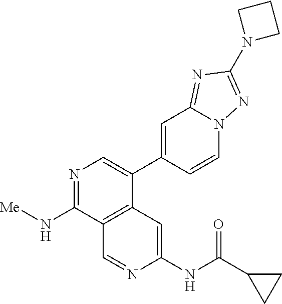 [SMILES: CNc1ncc(-c2ccn3nc(N4CCC4)nc3c2)c2cc(NC(=O)C3CC3)ncc12]

To a mixture of N-(5-(2-bromo-[1,2,4]triazolo[1,5-a]pyridin-7-yl)-8-(methylamino)-2,7-naphthyridin-3-yl)cyclopropanecarboxamide (40.0 mg; 0.091 mmol; 1.00 eq.) and in 1,4-dioxane (2.5 mL) was added Pd(DtBPF)Cl₂ (6 mg; 0.009 mmol; 0.10 eq.), Cs₂CO₃ (59.5 mg; 0.183 mmol; 2.00 eq.) and azetidine (21 mg, 0.36 mmol, 4 eq.). The reaction mixture was stirred for 3 h at 100° C. under nitrogen atmosphere. The desired product was observed via LCMS. The resulting mixture was concentrated under reduced pressure. The residue was purified by flash chromatography on silica gel column using MeOH/CH₂Cl₂ (0-20%) as eluent to afford an off-white crude solid. The residue was dissolved in DMSO (3 L). The precipitated solids were collected by filtration and washed with water (3×5 mL) to provide N-(5-(2-(azetidin-1-yl)-[1,2,4]triazolo[1,5-a]pyridin-7-yl)-8-(methylamino)-2,7-naphthyridin-3-yl)cyclopropanecarboxamide as an off-white solid (13.0 mg, 34%). LCMS (ESI) m/z 415.2, [M+H]⁺. ¹H NMR (400 MHz, DMSO-d₆) 10.99 (s, 1H), 9.40 (s, 1H), 8.74-8.70 (m, 1H), 8.36 (s, 1H), 8.14-8.09 (m, 1H), 8.05 (s, 1H), 7.46-7.44 (m, 1H), 7.01-6.98 (m, 1H), 4.08-4.00 (m, 4H), 3.02 (d, J=4.4 Hz 3H), 2.43-2.32 (m, 2H), 2.06-1.97 (m, 1H), 0.82-0.74 (m, 4H).

### Example 157: Synthesis of N-(8-(methylamino)-5-(4-phenyloxazol-2-yl)-2,7-naphthyridin-3-yl)cyclopropanecarboxamide

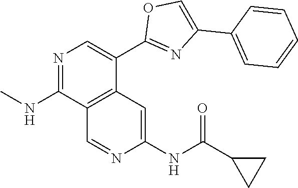 [SMILES: CNc1ncc(-c2nc(-c3ccccc3)co2)c2cc(NC(=O)C3CC3)ncc12]

### Step 1: 2-chloro-4-phenyloxazole

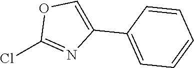 [SMILES: Clc1nc(-c2ccccc2)co1]

To a stirring mixture of 4-phenyloxazole (100 mg; 0.679 mmol; 1.00) eq.) in THF (4 mL) was added a solution of LiHMDS (1 M) in Tiff (0.82 mL; 0.815 mmol; 1.20 eq.) at −78° C. and stirred for 1 h at −0.78° C. under nitrogen atmosphere. To this reaction mixture was added a solution of perchloroethane in THF (322 mg; 1.36 mmol; 2.00 eq., 0.5 mL THF) was added at −0.78° C. and stirred for 0.5 h. Then reaction mixture was stirred for 0.5 h at room temperature. The reaction was quenched with a saturated ammonium chloride aqueous solution (20) mL). The reaction mixture was extracted with EtOAc (3×25 mL). The combined organic layers were washed with a saturated sodium chloride solution (2×10 mL), dried over anhydrous Na₂SO₄. After filtration, the filtrate was concentrated under reduced pressure. The residue was purified flash chromatography on silica gel column using 0-20% of EtOAc in petroleum ether as eluent to provide 2-chloro-4-phenyloxazole as a yellow solid (73.2 mg, 59%). LCMS (ESI) m/z 180.0, [M+H]⁺.

### Step 2: N-(8-(methylamino)-5-(4-phenyloxazol-2-yl)-2,7-naphthyridin-3-yl)cyclopropanecarboxamide

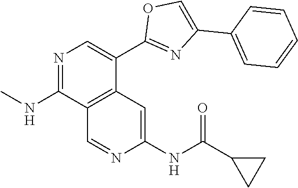 [SMILES: CNc1ncc(-c2nc(-c3ccccc3)co2)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (60 mg; 0.163 mmol; 1.00 eq.), 2-chloro-4-phenyloxazole (29.3 mg; 0.163 mmol; 1.00 eq.), XPhos Pd G₃ (13.8 mg; 0.016 mmol; 0.10 eq.), XPhos (7.78 mg; 0.016 mmol; 0.10 eq.) and K₃PO₄ (104 mg; 0.490 mmol; 3.00 eq.) in 1,4-dioxane/water (6:1, 3 mL) was stirred for 2 h at 90° C. The desired product was observed via LCMS. The resulting mixture was concentrated under reduced pressure. The residue was purified flash chromatography on silica gel column using 0-20% of MeOH in CH₂Cl₂ as eluent to provide the crude product. The crude product was purified by flash chromatography on pre-packed C18 column using 20-50% of MeCN in water (10 mmol/L NH₄HCO₃) as eluent to provide N-(8-(methylamino)-5-(5-methyloxazol-2-yl)-2,7-naphthyridin-3-yl)cyclopropanecarboxamide as an off-white solid (19.7 mg, 31%), LCMS (ESI) m/z 386.2, [M+H]⁺. ¹H NMR (400 MHz, DMSO-d₆) δ 11.02 (s, 1H), 9.88 (s, 1H), 9.42 (s, 1H), 8.74 (s, 1H), 8.66 (s, 1H), 8.49-8.46 (m, 1H), 8.03-7.99 (m, 2H), 7.50-7.31 (m, 3H), 3.07 (d, J=4.4 Hz, 3H), 2.13-2.09 (m, 11-1), 0.99-0.82 (m, 4H).

### Example 158: Synthesis of N-(8-(methylamino)-5-(5-phenyloxazol-2-yl)-2,7-naphthyridin-3-yl)cyclopropanecarboxamide

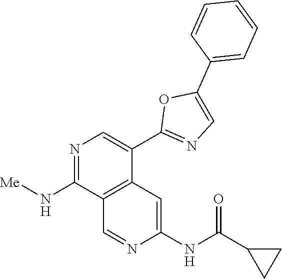 [SMILES: CNc1ncc(-c2ncc(-c3ccccc3)o2)c2cc(NC(=O)C3CC3)ncc12]

### Step 1: 2-chloro-5-phenyloxazole

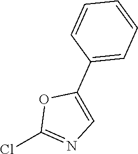 [SMILES: Clc1ncc(-c2ccccc2)o1]

To a stirring mixture of 5-phenyloxazole (100 mg; 0.689 mmol; 1.00 eq.) in THF (5 mL) was added a solution of LiHMDS (1 M) in THF (0.83 mL; 0.83 mmol; 1.20 eq.) at −78 T. The reaction was stirred at −78° C. under nitrogen atmosphere for 1 hour. To this reaction mixture was added a solution of perchloroethane in THF (322.8 mg; 1.364 mmol; 1.98 eq.; in 0.5 mL THF) at −78° C. and stirred for 0.5 h. Then the reaction was stirred at room temperature for 0.5 h. Upon completion, the reaction was quenched with a saturated NH₄Cl solution (10 mL), then extracted with EtOAc (3×10 mL), the combined organic phase was washed with a saturated NaCl solution (1×5 mL), The solution was dried with Na₂SO₄, filtered and the solvent was removed under reduced pressure. The residue was purified by flash chromatography on silica gel column using 1-10% of EtOAc in petroleum ether as eluent to provide 2-chloro-5-phenyloxazole as a yellow oil (90 mg, 72%). LCMS (ESI) m/z 180.0, [M+H]⁺.

### Step 2: N-(8-(methylamino)-5-(5-phenyloxazol-2-yl)-2,7-naphthyridin-3-yl)cyclopropanecarboxamide

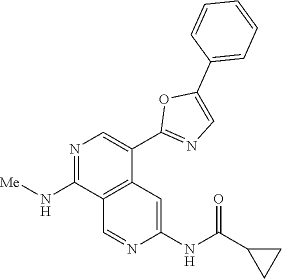 [SMILES: CNc1ncc(-c2ncc(-c3ccccc3)o2)c2cc(NC(=O)C3CC3)ncc12]

A mixture of K₃PO₄ (259.2 mg; 1.221 mmol; 3.00 eq.), XPhos Pd G₃ (34.5 mg; 0.041 mmol; 0.10 eq.), XPhos (19.4 mg; 0.041 mmol; 0.10 eq.) and N-(8-(methylamino)-5-(4,4,5,5-tetramethyl-1,3,2-dioxaborolan-2-yl)-2,7-naphthyridin-3-yl)cyclopropanecarboxamide (150 mg; 0.407 mmol; 1.00 eq.) in 1,4-dioxane/water (5:1, 6 mL) under N₂ atmosphere. To this mixture was added 2-chloro-5-phenyloxazole (73.0 mg; 0.406 mmol; 1.00 eq.). The reaction was stirred at 90° C. for 1 h. The mixture was allowed to cool to room temperature and the solvent was concentrated under reduced pressure. The residue was purified by flash chromatography on silica gel column using 1-10% of MeOH in CH₂Cl₂ as eluent to provide the crude product. The crude product was purified by flash chromatography on pre-packed C18 column using 20-0% MeCN in water (10 mmol/L NH₄HCO₃) as eluent to provide N-(8-(methylamino)-5-(5-phenyloxazol-2-yl)-2,7-naphthyridin-3-yl)cyclopropanecarboxamide as a yellow solid (38.4 mg, 24%). LCMS (ESI) m/z 386.2, [M+H]⁺. ¹H NMR (400 MHz, DMSO-d₆) δ 11.05 (s, 1H), 9.60 (s, 1H), 9.42 (s, 1H), 8.89 (s, 1H), 8.53-8.45 (m, 1H), 7.97-7.91 (m, 2H), 7.84 (s, 1H), 7.51-7.34 (m, 3H), 3.07 (d, 4.4 Hz, 3H), 2.16-2.06 (m, 1H), 0.97-0.82 (m, 4H).

### Example 159: Synthesis of N-(8-(methylthio)-5-phenyl-2,7-naphthyridin-3-yl)cyclopropanecarboxamide

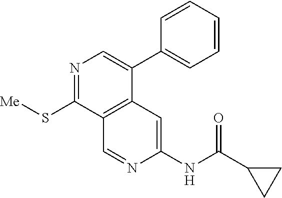 [SMILES: CSc1ncc(-c2ccccc2)c2cc(NC(=O)C3CC3)ncc12]

To a stirring mixture of N-(8-chloro-5-phenyl-2,7-naphthyridin-3-yl)cyclopropanecarboxamide (25 mg; 0.077 mmol; 1.00 eq.) in THF (1.5 mL) was added to sodium methanethiolate (21.6 mg; 0.308 mmol; 3.99 eq.). The reaction was stirred at room temperature for overnight. 92% desired product was detected on LCMS. The resulting mixture was concentrated under reduced pressure. The residue was purified by flash chromatography on pre-packed C18 column using 10-65% of MeCN in water (10 mmol/L NH₄. HCO₃) as eluent to provide N-(8-(methylthio)-5-phenyl-2,7-naphthyridin-3-yl)cyclopropanecarboxamide as a off-white solid (16.7 mg, 64%). LCMS (ESI) m/z 336.1, [M+H]⁺. ¹H NMR (400 MHz, DMSO-d₆) δ 11.19 (s, 1H), 9.40 (s, 1H), 8.48 (s, 1H), 8.36 (5, 1H), 7.58-7.48 (m, 5H), 2.72 (s, 3H), 2.10-2.00 (m, 1H), 0.85-0.74 (m, 4H).

### Example 160: Synthesis of N-(8-((2-hydroxyethyl)amino-5-phenyl-2,7-naphthyridin-3-yl)cyclopropanecarboxamide

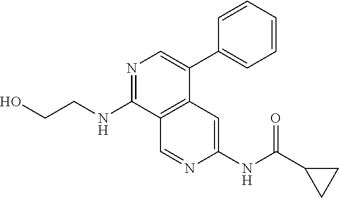 [SMILES: O=C(Nc1cc2c(-c3ccccc3)cnc(NCCO)c2cn1)C1CC1]

### Step 1: N-(8-((2-((tert-butyldimethylsilyl)oxy)ethyl)amino)-5-phenyl-2,7-naphthyridin-3-yl)cyclopropanecarboxamide

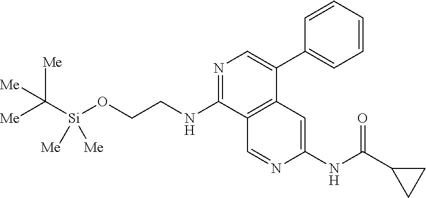 [SMILES: CC(C)(C)[Si](C)(C)OCCNc1ncc(-c2ccccc2)c2cc(NC(=O)C3CC3)ncc12]

To a mixture of N-(8-chloro-5-phenyl-2,7-naphthyridin-3-yl)cyclopropanecarboxamide (35 mg; 0.108 mmol; 1.00 eq.) and 2-((tert-butyldimethylsilyl)oxy)ethan-1-amine (57 mg; 0.325 mmol; 3.01 eq.) in 1,4-dioxane (3 mL) was added Pd-PEPPSI-IHeptCl (10.5 mg; 0.011 mmol; 0.10 eq.) and Cs₂CO₃ (70 mg; 0.215 mmol; 1.99 eq.). The reaction was stirred for 2 h at 90° C. under nitrogen. Upon completion, this reaction was concentrated under vacuum. The residue was purified by flash chromatography on silica gel column using 40-50% of EtOAc in petroleum ether as eluent to afford N-(8-((2-((tert-butyldimethylsilyl)oxy)ethylamino)-5-phenyl-2,7-naphthyridin-3-yl cyclopropanecarboxamide as a white solid (30 mg, 59%). LCMS (ESI) m/z 463.2, [M+H]⁺.

### Step 2: N-(8((2-hydroxyethyl)amino)-5-phenyl-2,7-naphthyridin-3-yl)cyclopropanecarboxamide

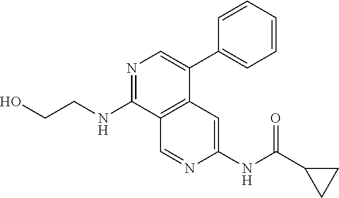 [SMILES: O=C(Nc1cc2c(-c3ccccc3)cnc(NCCO)c2cn1)C1CC1]

To a stirring mixture of N-(8-((2-((tert-butyldimethylsilyl)oxy)ethylamino)-5-phenyl-2,7-naphthyridin-3-yl)cyclopropanecarboxamide (30 mg, 0.065 mmol; 1.00 eq.) in CH₂Cl₂ (1 mL) was added a solution of HCl in dioxane (4 M, 2 mL) and stirred at 25° C. for 1 hour. The solvent was concentrated under vacuum. The residue was purified by flash chromatography on pre-packed C18 column using 50-80% MeCN in water (10 mmol/L NH₄HCO₃) as eluent to provide N-(8-((2-hydroxyethyl)amino)-5-phenyl-2,7-naphthyridin-3-yl)cyclopropanecarboxamide as a white solid (13.4 mg, 59%). LCMS (ESI) m/z 349.2, [M+H]⁺. ¹H NMR (400 MHz, DMSO-d₆) δ 10.95 (s, 1H), 9.44 (s, 1H), 8.34 (s, 7.98-7.92 (m, 1H), 7.90 (s, 1H), 7.53-7.46 (m, 2H), 7.44-7.37 (m, 3H), 4.80 (t, J=5.2 Hz, 1H), 3.70-3.59 (m, 4H), 2.05-1.98 (m, 1H), 0.83-0.74 (m, 4H).

### Example 161: Synthesis of 1-fluoro-N-(8-(methylamino)-5-phenyl-2,7-naphthyridin-3-yl)cyclopropane-1-carboxamide

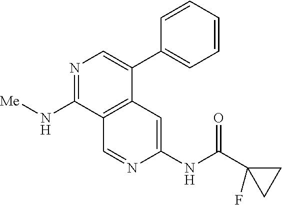 [SMILES: CNc1ncc(-c2ccccc2)c2cc(NC(=O)C3(F)CC3)ncc12]

### Step 1: N-(2,4-dimethoxybenzyl)-1-fluorocyclopropane-1-carboxamide

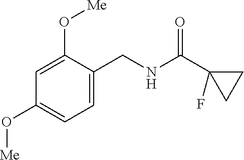 [SMILES: COc1ccc(CNC(=O)C2(F)CC2)c(OC)c1]

To a stirring mixture of 1-fluorocyclopropane-1-carboxylic acid (200 mg; 1.92 mmol; 1.00 eq.) in DMF (2.0 mL) was added HATU (877 mg; 2.31 mmol; 1.20 eq.) and stirred for 20 min at room temperature under nitrogen atmosphere followed by the addition of DIPEA (744 mg; 5.76 mmol; 3.00 eq.) and (2,4-dimethoxyphenyl)methanamine (642 mg; 3.84 mmol; 2.00 eq.) in portions. The reaction mixture was stirred for 3 h at room temperature. The mixture was diluted with EtOAc (100 mL), washed with brine (3×10 mL), dried over anhydrous Na₂SO₄. After filtration, the filtrate was concentrated under reduced pressure. The crude product mixture was purified by CIS column using 20-80% of MeCN in water (10 mmol/L NH₄HCO₃) as eluent to provide N-(2,4-dimethoxybenzyl)-1-fluorocyclopropane-1-carboxamide as a yellow oil (410 mg, 83%). LCMS (ESI) m/z 254.1, [M+H]⁺.

### Step 2: 1-fluorocyclopropane-1-carboxamide

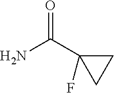

N-(2,4-dimethoxybenzyl)-1-fluorocyclopropane-1-carboxamide (410 mg; 1.25 mmol; 1.00 eq.) was dissolved in TFA (3.0 mL). The resulting mixture was stirred for 1 h at 70° C. under nitrogen atmosphere. Upon completion, the resulting mixture was concentrated under reduced pressure. The residue was purified by flash chromatography on silica gel column using MeOH/CH₂Cl₂ (0-20%) of as eluent to provide 1-fluorocyclopropane-1-carboxamide as a purple solid (180 mg, crude). LCMS (ESI) m/z 104.0, [M+H]⁺.

### Step 3: 1-fluoro-N-(8-(methylamino-5-phenyl-2,7-naphthyridin-3-yl)cyclopropane-1-carboxamide

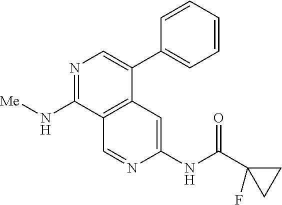 [SMILES: CNc1ncc(-c2ccccc2)c2cc(NC(=O)C3(F)CC3)ncc12]

A mixture of 1-fluorocyclopropane-1-carboxamide (38.2 mg; 0.371 mmol; 2.00 eq.), 6-chloro-N-methyl-4-phenyl-2,7-naphthyridin-1-amine (50.0 mg; 0.185 mmol; 1.00 eq.), Pd₂(dba)₃ (17.0 mg; 0.019 mmol; 0.10 eq.), XantPhos (21.5 mg; 0.037 mmol; 0.20 eq.) and Cs₂CO₃ (121 mg; 0.371 mmol; 2.00 eq.) in 1,4-dioxane (3.0 mL) was stirred for 1 h at 130° C. under nitrogen atmosphere. The desired product was observed by LCMS. The resulting mixture was concentrated under reduced pressure. The residue was purified by flash chromatography on silica gel column using 0-20% of MeOH in CH₂Cl₂ as eluent to afford a brown crude solid. The crude product was further purified by C18 column using 40-70% of MeCN in water (10 mmol/L, NH₄HCO₃) as eluent to provide 1-fluoro-N-(8-(methylamino)-5-phenyl-2,7-naphthyridin-3-yl)cyclopropane-1-carboxamide as as a white solid (31.3 mg, 50%). LCMS (ESI) m/z 337.1, [M+H]⁺. ¹H NMR (400 MHz, DMSO-d₆) δ 10.32 (s, 1H), 9.44 (s, 1H), 8.31 (s, 1H), 8.11-8.07 (m, 1H), 7.98 (s, 1H), 7.51-7.38 (m, 5H), 3.03 (d, J=4.4 Hz, 3H), 1.49-1.29 (m, 4H).

### Example 162: Synthesis of N⁶-isopropyl-N¹-methyl-4-phenyl-2,7-naphthyridine-1,6-diamine

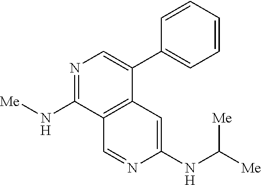 [SMILES: CNc1ncc(-c2ccccc2)c2cc(NC(C)C)ncc12]

To a stirring mixture of 6-chloro-N-methyl-4-phenyl-2,7-naphthyridin-1-amine (50 mg; 0.185 mmol; 1.00 eq.) in 1,4-dioxane (2 mL) was added to Pd-PEPPSI-IHeptCl (18 mg; 0.018 mmol; 0.10 eq.) and t-BuOK (41.6 mg; 0.371 mmol; 2.00 eq.) under N₂ atmosphere. To this reaction mixture was added propan-2-amine (43.8 mg; 0.741 mmol; 4.00 eq.). The reaction was stirred at 90° C. for 2 h. 47% desired product was detected on LCMS. The reaction mixture was filtered, and the filtrate was concentrated under pressure. The residue was purified by flash chromatography on silica gel column using MeOH in CH₂Cl₂ (1-8%) as eluent to provide the crude product. The crude product was purified by flash chromatography on pre-packed C18 column using 10-55% of MeCN in water (10 mmol/L NH₄HCO₃) as eluent to provide N⁶-isopropyl-N¹-methyl-4-phenyl-2,7-naphthyridine-1,6-diamine as a off-white solid (14.4 mg, 26%). LCMS (ESI) m/z 293.2, [M+H]⁺. ¹H NMR (400 MHz, DMSO-d₆) δ 9.10 (s, 1H), 7.66 (s, 1H), 7.61-7.56 (m, 1H), 7.51-7.32 (m, 5H), 6.62. (d, J=7.6 Hz, 1H), 6.36 (s, 1H), 4.00-3.88 (m, 1H), 2.96 (d, J=4.4 Hz, 3H), 1.11 (d, J=6.4 Hz, 6H).

### Example 163: Synthesis of N-(6-methyl-8-(methylamino)-2,7-naphthyridin-3-yl)cyclopropanecarboxamide

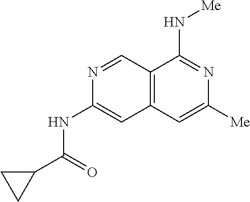 [SMILES: CNc1nc(C)cc2cc(NC(=O)C3CC3)ncc12]

### Step 1: 1-(tert-butyl) 3-methyl 2-(2-chloro-5-(methoxycarbonyl)pyridin-4-yl)malonate

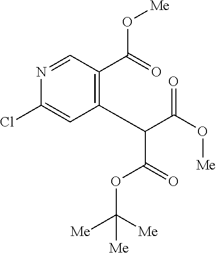 [SMILES: COC(=O)c1cnc(Cl)cc1C(C(=O)OC)C(=O)OC(C)(C)C]

A mixture of methyl 4,6-dichloronicotinate (5 g; 24.26 mmol; 1.00 eq.) and tert-butyl methyl malonate (6.5 g; 37.314 mmol; 1.54 eq.) in DMF (50 mL) was added Cs₂CO₃ (16 g; 49.10 mmol; 2.02 eq.) under nitrogen atmosphere. The resulting solution was stirred for 3 h at 40° C. The desired product was observed via LCMS. The resulting mixture was diluted with water (250 mL). The resulting solution was extracted with EtOAc (100 mL 2) and the organic layers were combined. The organic layer was dried over anhydrous sodium sulfate and concentrated under vacuum to provide 1-(tert-butyl) 3-methyl 2-(2-chloro-5-(methoxycarbonyl)pyridin-4-yl)malonate as a yellow oil (10.7 g, crude). LCMS (ESI) m/z 344.1, [M+H]⁺.

### Step 2: methyl 6-chloro-4-(2-methoxy-2-oxoethyl)nicotinate

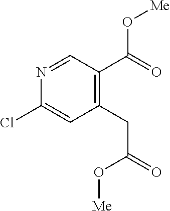 [SMILES: COC(=O)Cc1cc(Cl)ncc1C(=O)OC]

A mixture of 1-(tert-butyl) 3-methyl 2-(2-chloro-5-(methoxycarbonyl)pyridin-4-yl)malonate (10 g; 30.32 mmol; 1.00 eq.) in CH₂Cl₂ (50 mL) was added TFA (20 mL) under nitrogen atmosphere. The resulting solution was stirred for 12 h at room temperature. The reaction was concentrated under vacuum. The reaction mixture was diluted with water (300 mL). The resulting solution was adjusted pH to 7 and extracted with EtOAc (200 mL×2) and the organic layers were combined. The organic layer was dried over anhydrous sodium sulfate and concentrated under vacuum to provide methyl 6-chloro-4-(2-methoxy-2-oxoethyl)nicotinate as a off-white solid (5 g, crude). LCMS (ESI) m/z 244.0, [M+H]⁺.

### Step 3: 6-chloro-2,7-naphthyridine-1,3(2H,4H)-dione

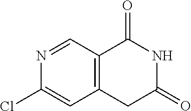 [SMILES: O=C1Cc2cc(Cl)ncc2C(=O)N1]

Methyl 6-chloro-4-(2-methoxy-2-oxoethyl)nicotinate (1 g; 2.052 mmol; 1.00 eq.) was dissolved in a solution of NH₃·H₂O (5 mL). The resulting mixture was stirred at 120° C. in a sealed tube for 12 hours. The reaction was cooled to rt. After filtration, the solid was washed with water and dried to provide 6-chloro-2,7-naphthyridine-1,3(2H,4H)-dione as a brown solid (300 mg, crude). LCMS (ESI) m/z 197.0, [M+H]⁺. ¹⁻H NMR (400 MHz, DMSO-d₆) δ 9.65 (s, 1H), 8.27 (s, 1H), 6.56 (s, 1H), 4.73 (s, 2H).

### Step 4: N-(6,8-dioxo-5,6,7,8-tetrahydro-2,7-naphthyridin-3-yl)cyclopropanecarboxamide

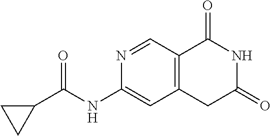 [SMILES: O=C1Cc2cc(NC(=O)C3CC3)ncc2C(=O)N1]

A mixture of Pd₂(dba)₃, (303 mg; 0.331 mmol; 0.10 eq.), XantPhos (383 mg; 0.662 mmol; 0.20 eq.) and Cs₂CO₃ (2.16 g; 663 mmol; 2.00 eq.) in 1,4-dioxane (30 mL) was added cyclopropanecarboxamide (1.12 g; 13.1 mmol; 4.00 eq.) and 6-chloro-2,7-naphthyridine-1,3(2H,4H)-dione (650 mg; 3.31 mmol; 1.00 eq.) under N₂ atmosphere. The resulting solution was stirred for 48 h at 130° C. Upon completion, this reaction was concentrated under vacuum. The residue was purified by flash chromatography on silica gel column using MeOH/CH₂Cl₂ (10-20%) as eluent to provide N-(6,8-di oxo-5,6,7,8-tetrahydro-2,7-naphthyridin-3-yl)cyclopropanecarboxamide as a brown solid (500 mg, 61%). LCMS (ESI) m/z 246.1, [M+H]⁺. ¹H NMR (400 MHz, DMSO-d₆) δ 11.28 (s, 1H), 11.19 (s, 1H), 8.86 (s, 1H), 8.06 (s, 1H), 4.04 (s, 2H), 2.12-1.96 (m, 1H), 0.94-0.74 (m, 4H).

### Step 5: 6-(cyclopropanecarboxamido)-2,7-naphthyridine-1-diyl bis(trifluoromethanesulfonate)

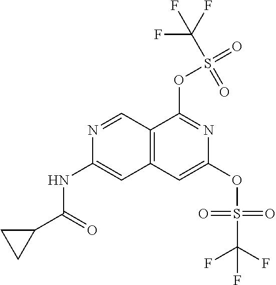 [SMILES: O=C(Nc1cc2cc(OS(=O)(=O)C(F)(F)F)nc(OS(=O)(=O)C(F)(F)F)c2cn1)C1CC1]

To a stirring mixture of N-(6,8-dioxo-5,6,7,8-tetrahydro-2,7-naphthyridin-3-yl)cyclopropanecarboxamide (400 mg; 1.63 mmol; 1.00 eq.) and pyridine (400 mg; 5.057 mmol; 3.10 eq.) in DCE (40 mL) was added a solution of Tf₂O (1.4 g; 4.96 mmol; 3.00 eq.) in DCE (5 mL) at 0° C. under nitrogen atmosphere. The resulting solution was stirred for 1 hour at 0° C. Upon completion, the reaction was diluted with CH₂Cl₂ (100 mL), the organic phase was washed with a saturated NaCl solution (3×5 mL). The organic phase was dried with Na₂SO₄, filtered and the solvent was removed under reduced pressure. The residue was purified by flash chromatography on pre-packed C18 column using 70-80% MeCN in water (10 mmol/L NH₄HCO₃) as eluent to provide 6-(cyclopropanecarboxamido)-2,7-naphthyridine-1,3-diyl bis(trifluoromethanesulfonate) as a off-white solid (450 mg, 54%). LCMS (ESI) m/z 510.0, [M+H]⁺.

### Step 6: N-(6-methyl-8-(methylamino)-2,7-naphthyridin-3-yl)cyclopropanecarboxamide

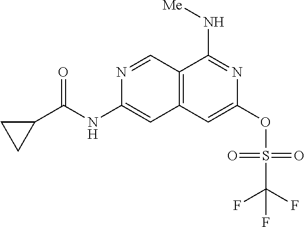 [SMILES: CNc1nc(OS(=O)(=O)C(F)(F)F)cc2cc(NC(=O)C3CC3)ncc12]

To a stirring mixture of 6-(cyclopropanecarboxamido)-2,7-naphthyridine-1,3-diyl bis(trifluoromethanesulfonate) (200 mg; 0.393 mmol 1.00 eq.) and methanamine hydrochloride (30 mg; 0.444 mmol; 1.13 eq.) in NMP (7 mL) was added DIPEA (100 mg; 0.774 mmol; 1.97 eq.) under nitrogen atmosphere. The resulting solution was stirred for 2 h at room temperature. Upon completion, the reaction was diluted with EtOAc (80 mL), the organic phase was washed with a saturated NaCl solution (3×5 mL). The organic phase was dried with Na₂SO₄, filtered and the solvent was removed under reduced pressure. The residue was purified by flash chromatography on silica gel column using 60-80% of EtOAc in petroleum ether as eluent to provide N-(6-methyl-8-(methylamino)-2,7-naphthyridin-3-yl)cyclopropanecarboxamide as a white solid (90 mg, 58%). LCMS (ESI) m/z 391.1 [M+H]⁺. ¹H NMR (400 MHz, DMSO-d₆) δ 11.09 (s, 1H), 9.37 (s, 1H), 8.64-8.58 (m, 1H), 8.29 (s, 1H), 683 (s, 1H), 2.94 (d, J=4.4 Hz, 3H), 2.10-2.01 (m, 1H), 0.88-0.81 (m, 4H).

### Step 7: N-(6-methyl-8-(methylamino)-2,7-naphthyridin-3-yl)cyclopropanecarboxamide

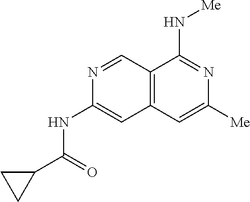 [SMILES: CNc1nc(C)cc2cc(NC(=O)C3CC3)ncc12]

To a stirring mixture of Pd(DtBPF)Cl₂ (11 mg; 0.017 mmol; 0.11 eq.) and K₃PO₄ (66 mg; 0.311 mmol, 2.02 eq.) in 1,4-dioxane/water (5:1, 6 mL) was added 2,4,6-trimethyl-1,3,5,2,4,6-trioxatriborinane (47 mg, 0.374 mmol; 2.44 eq.) and 6-(cyclopropanecarboxamido)-1-(methylamino)-2,7-naphthyridin-3-yl trifluoromethanesulfonate (60 mg; 0.154 mmol; 1 eq.) under nitrogen atmosphere. The resulting solution was stirred for 2 h at 90 The reaction was concentrated under vacuum. The reaction mixture was purified by flash chromatography on silica gel column using 60-80% of EtOAc in petroleum ether as eluent to afford the crude product and it was re-purified by flash chromatography on pre-packed C18 column using 70-80% MeCN in water (10 mmol/L NH₄HCO₃) as eluent to provide N-(6-methyl-8-(methylamino)-2,7-naphthyridin-3-yl)cyclopropanecarboxamide as a white solid (16.2 mg, 41%). LCMS (ESI) m/z 257.1 [M+H]⁺. ¹H NMR (400 MHz, DMSO-d₆) δ 10.85 (5, 1H), 9.22 (s, 1H), 8.05 (s, 1H), 7.81-7.73 (m, 1H), 6.57 (s, 1H), 2.95 (d, J=4.4 Hz, 3H), 2.34 (s, 3H), 2.11-1.98 (m, 1H), 0.89-0.78 (m, 4H).

### Example 164: Synthesis of N-(8-(methylamino)-6-vinyl-2,7-naphthyridin-3-yl)cyclopropanecarboxamide

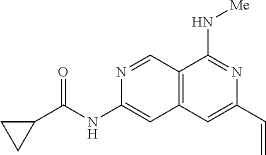 [SMILES: C=Cc1cc2cc(NC(=O)C3CC3)ncc2c(NC)n1]

A mixture of Pd(DtBPF)Cl₂ (8.4 mg; 0.013 mmol; 0.10 eq.) and K₃PO₄ (54 mg; 0.254 mmol; 1.99 eq.) in 1,4-dioxane/water (5:1, 4.8 mL) was added 4,4,5,5-tetramethyl-2-vinyl-1,3,2-dioxaborolane (24 mg; 0.156 mmol; 1.22 eq.), 6-cyclopropaneamido-1-(methylamino)-2,7-naphthyridin-3-yltrifluoromethanesulfonate (100 mg; 0.128 mmol; 1.00 eq.) under nitrogen atmosphere. The resulting solution was stirred for 2 h at 9° C. The reaction was concentrated under vacuum. The residue was purified by flash chromatography on silica gel column using 60-80% of EtOAc in petroleum ether as eluent to afford the crude product. The crude product was purified by flash chromatography on pre-packed C18 column using 70-80% MeCN in water (10 mmol/L, NH₄HCO₃) as eluent to provide N-(8-(methylamino)-6-vinyl-2,7-naphthyridin-3-yl)cyclopropanecarboxamide as a white solid (6.6 mg). LCMS (ESI)m/z 269.1, [M+H]⁺. ¹H-NMR (400 MHz, DMSO-d₆) δ 10.91 (s, 1H), 9.25 (s, 1H), 8.15 (s, 1H), 7.88-7.79 (m, 1H), 6.74 (s, 1H), 673-6.68 (m, 1H), 6.33 (dd, J=17.2, 2.4 Hz, 1H), 5.43 (dd, J=10.4, 2.4 Hz, 1H), 3.01 (d, J=4.4 Hz, 3H), 2.10-2.01 (m, 1H), 0.89-0.79 (m, 4H)

### Example 165: Synthesis of N-(6-ethyl-8-(methylamino)-2,7-naphthyridin-3-yl)cyclopropanecarboxamide

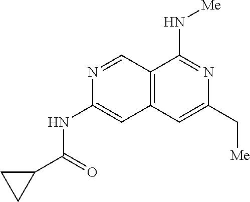 [SMILES: CCc1cc2cc(NC(=O)C3CC3)ncc2c(NC)n1]

To a stirring mixture of N-(8-(methylamino)-6-vinyl-2,7-naphthyridin-3-yl)cyclopropanecarboxamide (25 mg; 0.093 mmol; 1.00 eq.) in MeOH (3 mL) was added 10% Pd/C (25 mg; 100% w/w). The resulting solution was stirred for 3 h at 35° C. The desired product was observed via LCMS. After filtration, the filtrate was concentrated under vacuum. The residue was purified by flash chromatography on pre-packed C18 column using 70-80% MeCN in water (10 mmol/L NH₄HCO₃) as eluent to provide N-(6-ethyl-8-(methylamino)-2,7-naphthyridin-3-yl)cyclopropanecarboxamide as a white solid (15.5 mg, 61%), LCMS (ESI) m/z 271.1, [M+H]⁺. ¹H NMR (400 MHz, DMSO-d₄) δ 10.85 (s, 1H), 9.22 (s, 1H), 8.08 (s, 1H), 7.79-7.73 (m, 1H), 6.57 (s, 1H), 2.97 (d, J=4.4 Hz, 3H), 2.61 (q, J=7.6 Hz, 2H), 2.08-2.01 (m, 1H), 1.23 (t, 7.6 Hz, 3H), 0.88-0.79 (m, 4H).

### Example 166: Synthesis of N-(5-(4-(1H-pyrazol-1-yl)phenyl)-8-(methylamino)-2,7-naphthyridin-3-yl)cyclopropanecarboxamide

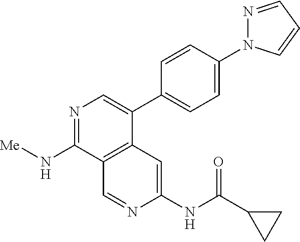 [SMILES: CNc1ncc(-c2ccc(-n3cccn3)cc2)c2cc(NC(=O)C3CC3)ncc12]

To a stirring mixture of N-(5-bromo-8-(methylamino)-2,7-naphthyridin-3-yl)cyclopropanecarboxamide (50 mg; 0.156 mmol; 1.00 eq.) in 1,4-dioxane/water (5:1, 2.4 mL), was added 1-(4-(4,4,5,5-tetramethyl-1,3,2-dioxaborolan-2-yl)phenyl)-1H-pyrazole (84.3 mg; 0.312 mmol; 2.00 eq.), Pd(DtBPF)Cl₂ (9.8 mg; 0.015 mmol; 0.10 eq.) and K₃PO₄ (66.2 mg; 0.312 mmol; 2.00 eq.) under nitrogen atmosphere. The reaction was stirred at 90° C. for 2 h. The desired product was observed via LCMS. The resulting mixture was concentrated under reduced pressure. The residue was purified by flash chromatography via a silica gel column using MeOH/CH₂Cl₂ (3-7%) as eluent to give crude product (34 mg). The product was re-purified by flash chromatography on pre-packed C18 column using 20-49% of MeCN in water (10 mmol/L NH₄HCO₃) as eluent to provide N-(5-(4-(1H-pyrazol-1-yl)phenyl)-8-(methylamino)-2,7-naphthyridin-3-yl)cyclopropanecarboxamide as a off-white solid (26.2 mg, 72%). LCMS (ESI) m/z 385.2, [M+H]⁺. ¹H NMR (400 MHz, DMSO-d₆) δ 10.95 (s, 1H), 9.39 (s, 1H), 8.59-8.57 (m, 1H), 8.38 (s, 1H), 8.02-7.93 (m, 4H), 7.79-7.77 (m, 1H), 7.54-7.52 (m, 2H), 6.59-6.57 (m, 1H), 3.02 (d, J=4.4 Hz, 3H), 2.05-1.99 (m, 1H), 0.81-0.74 (m, 4H).

### Examples 167-171

Each compound in Table 7 was prepared following the same experimental procedure that was previously described in the synthesis of compound in Example 166: using N-(5-bromo-8-(methylamino)-2,7-naplathyridin-3-yl)cyclopropanecarboxamide as the common intermediate and appropriate boronic ester or acid).

TABLE 7

| Example | Structure and Name | MS and ¹H NMR |
|---|---|---|
| Example 167 | 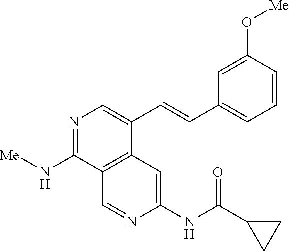 [SMILES: CNc1ncc(/C=C/c2cccc(OC)c2)c2cc(NC(=O)C3CC3)ncc12] | LCMS (ESI) m/z 375.2, [M + H]⁺. ¹H NMR (400 MHz, DMSO-d₆) δ 11.03 (s, 1H), 9.36 (s, 1H), 8.57 (s, 1H), 8.33 (s, 1H), 8.09-8.01 (m, 1H), 7.41-7.29 (m, 2H), 7.18-7.10 (m, 2H), 7.04 (d, J = 16.0 Hz, 1H), 6.90-6.82 (m, 1H), 3.80 (s, 3H), 3.01 (d, J = 4.4 Hz, 3H), 2.12- 2.04 (m, 1H), 0.92-0.81 (m, 4H). |
| | (E)-N-(5-(3-methoxystyryl)-8- | |
| | (methylamino)-2,7- | |
| | naphthyridin-3- | |
| | yl)cyclopropanecarboxamide | |
| Example 168 | 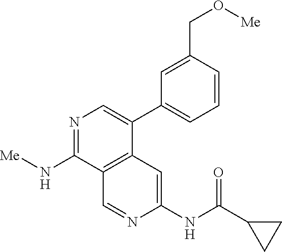 [SMILES: CNc1ncc(-c2cccc(COC)c2)c2cc(NC(=O)C3CC3)ncc12] | LCMS (ESI) m/z 363.2, [M + H]⁺. ¹H NMR (400 MHz, DMSO-d₆) δ 10.93 (s, 1H), 9.38 (s, 1H), 8.32 (s, 1H), 8.02- 7.94 (m, 1H), 7.92 (s, 1H), 7.49-7.43 (m, 1H), 7.37-7.30 (m, 3H), 4.47 (s, 2H), 3.32 (s, 3H), 3.01 (d, J = 4.4 Hz, 3H), 2.06-1.97 (m, 1H), 0.83-0.74 (m, 4H). |
| | N-(5-(3- | |
| | (methoxymethyl)phenyl)-8- | |
| | (methylamino)-2,7- | |
| | naphthyridin-3- | |
| | yl)cyclopropanecarboxamide | |
| Example 169 | 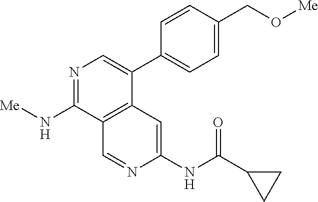 [SMILES: CNc1ncc(-c2ccc(COC)cc2)c2cc(NC(=O)C3CC3)ncc12] | LCMS (ESI) m/z 363.2, [M + H]⁺. ¹H NMR (400 MHz, DMSO-d₆) δ 10.93 (s, 1H), 9.38 (s, 1H), 8.36 (s, 1H), 7.99-7.95 (m, 1H), 7.93 (s, 1H), 7.46-7.36 (m, 4H), 4.48 (s, 2H), 3.36 (s, 3H), 3.01 (d, J = 4.4 Hz, 3H), 2.07-1.96 (m, 1H), 0.81-0.74 (m, 4H). |
| | N-(5-(4- | |
| | (methoxymethyl)phenyl)-8- | |
| | (methylamino)-2,7- | |
| | naphthyridin-3- | |
| | yl)cyclopropanecarboxamide | |
| Example 170 | 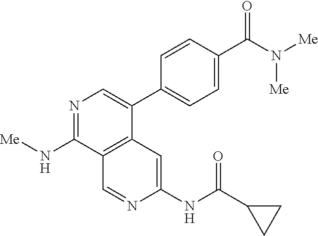 [SMILES: CNc1ncc(-c2ccc(C(=O)N(C)C)cc2)c2cc(NC(=O)C3CC3)ncc12] | LCMS (ESI) m/z 390.2, [M + H]⁺. ¹H NMR (400 MHz, DMSO-d₆) δ 10.94 (s, 1H), 9.39 (s, 1H), 8.37 (s, 1H), 8.06- 8.00 (m, 1H), 7.98 (s, 1H), 7.56-7.46 (m, 4H), 3.02 (d, J = 4.4 Hz, 9H), 2.07- 1.98 (m, 1H), 0.84-0.73 (m, 4H). |
| | 4-(6- | |
| | (cyclopropanecarboxamido)-1- | |
| | (methylamino)-2,7- | |
| | naphthyridin-4-yl)-N,N- | |
| | dimethylbenzamide | |
| Example 171 | 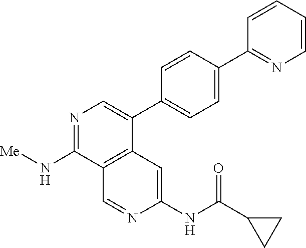 [SMILES: CNc1ncc(-c2ccc(-c3ccccn3)cc2)c2cc(NC(=O)C3CC3)ncc12] | LCMS (ESI) m/z 396.2, [M + H]⁺. ¹H NMR (400 MHz, DMSO-d₆) δ 10.96 (s, 1H), 9.40 (s, 1H), 7.72-7.69 (m, 1H), 8.43 (s, 1H), 8.24-8.18 (m, 2H), 8.09-7.98 (m, 3H), 7.96-7.87 (m, 1H), 7.58-7.52 (m, 2H), 7.42-7.34 (m, 1H), 3.03 (d, J = 4.4 Hz, 3H), 2.07-1.97 (m, 1H), 0.81-0.74 (m, 4H). |
| | N-(8-(methylamino)-5-(4- | |
| | (pyridin-2-yl)phenyl)-2,7- | |
| | naphthyridin-3- | |
| | yl)cyclopropanecarboxamide | |

### Example 172: Synthesis of N-(5-(3-((2-methoxyethoxy)methyl)phenyl)-8-(methylamino)-2,7-naphthyridin-3-yl)cyclopropanecarboxamide

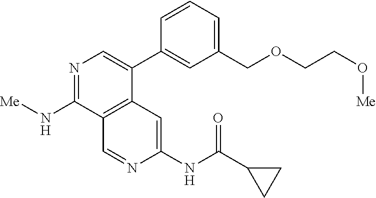 [SMILES: CNc1ncc(-c2cccc(COCCOC)c2)c2cc(NC(=O)C3CC3)ncc12]

### Step 1: 1-promo-3-((2-methoxyethoxy)methyl)benzene

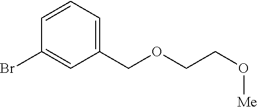 [SMILES: COCCOCc1cccc(Br)c1]

To a solution of (3-bromophenyl)methanol (500 mg; 2.673 mmol; 1.00 eq.) in DMF (5 mL) was treated with NaH (324.8 mg; 14.128 mmol; 5.28 eq.) at 0° C. and stirred for 0.5 h at room temperature under nitrogen atmosphere followed by the addition of 1-bromo-2-methoxyethane (743.1 mg; 5.346 mmol; 2.00 eq.). The resulting mixture was stirred for an additional of 3 h at room temperature. The desired product was observed via LCMS. The reaction was quenched with a sat. NH₄Cl solution at 0° C. The resulting mixture was extracted with EtOAc (3×30 mL). The combined organic layers were washed with brine (10×2 mL), dried over anhydrous Na₂SO₄. After filtration, the filtrate was concentrated under reduced pressure. The residue was purified by flash chromatography on silica gel column using 10-80% of CH₂Cl₂ in petroleum ether as eluent to provide 1-bromo-3-((2-methoxyethoxy)methyl)benzene as a colorless oil (532.5 mg, 81%).

### Step 2: N-(5-(3-((2-methoxyethoxy)methyl)phenyl)-8-(methylamino)-2,7-naphthyridin-3-yl)cyclopropanecarboxamide

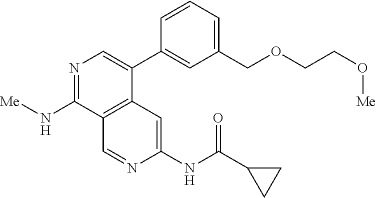 [SMILES: CNc1ncc(-c2cccc(COCCOC)c2)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (60.1 mg; 0.187 mmol; 1.00 eq.) and 1-bromo-3-((2-methoxyethoxy)methyl)benzene (49.7 mg; 0.187 mmol; 1.00 eq.), Pd(DtBPF)Cl₂ (12.3 mg; 0.019 mmol; 0.10 eq.) and K₃PO₄ (79.2 mg; 0.373 mmol; 2.00 eq.) in 1,4-dioxane/water (5:1, 3 mL) was stirred for 2 h at 90° C. under nitrogen atmosphere. The desired product could be detected by LCMS. The resulting mixture was concentrated under reduced pressure. The residue was purified by flash chromatography on silica gel column using MeOH/CH₂Cl₂ (2-10%) as eluent to provide crude product. The crude product was re-purified by flash chromatography on pre-packed C18 column using 20-50% of MeCN in water (10 mmol/1. NH₄HCO₃) as eluent to provide 36.7 mg of crude product. The product was re-purified by reverse phase preparative HPLC (Prep-C18, XBridge Prep OBD C18 Column, 30×150 mm, waters; gradient elution of 49-57% MeCN in water over a 8 min period, where both water and MeCN contain 10 mmol/L NH₄HCO₃, flow rate: 60 mL/min, detector UV wavelength: 254 nm) to afford N-(5-(3-((2-methoxyethoxy)methyl)phenyl)-8-(methy amino)-2,7-naphthyridin-3-yl)cyclopropanecarboxamide as a yellow solid (15.9 mg, 22%). LCMS (ESI) m/z 407.2, [M+H]⁺. ¹H NMR (400 MHz, DMSO-d₆) δ 10.92 (s, 1H), 9.38 (m, 1H), 8.33 (s, 1H), 8.00-7.96 (m, 1H), 7.92 (s, 1H), 7.49-7.43 (m, 1H), 7.38-7.30 (m, 3H), 4.55 (s, 2H), 3.65-3.57 (m, 2H), 3.54-3.47 (m, 2H), 3.24 (s, 3H), 3.01 (d, 4.4 Hz, 3H), 2.06-1.97 (m, 1H), 0.83-0.72 (m, 4H).

### Example 173: Synthesis of N-(5-(4-(1-methyl-1H-pyrazol-5-yl)phenyl)-8-(methylamino)-2,7-naphthyridin-3-yl)cyclopropanecarboxamide

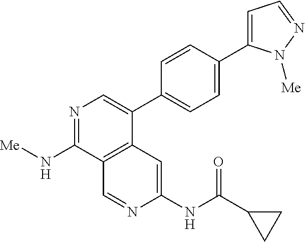 [SMILES: CNc1ncc(-c2ccc(-c3ccnn3C)cc2)c2cc(NC(=O)C3CC3)ncc12]

To a stirring mixture of N-(8-(methylamino)-5-(4,4,5,5-tetramethyl-1,3,2-dioxaborolan-2-yl)-2,7-naphthyridin-3-yl)cyclopropanecarboxamide (100 mg; 0.272 mmol; 1.00 eq.) and 5-(4-bromophenyl)-1-methylpyrazole (64.3 mg; 0.271 mmol; 1.00 eq.) in 1,4-dioxane/water (5:1, 3 mL) was added XPhos Pd G₃ (22.9 mg; 0.027 mmol; 0.10 eq.) and XPhos (12.9 mg; 0.027 mmol; 0.10 eq.) and K₃PO₄ (172.9 mg; 0.815 mmol; 3.00 eq.). The resulting solution was stirred for 1 h at 90° C. under nitrogen atmosphere. The desired product was observed via. LCMS. The mixture was allowed to cool down to room temperature. The resulting mixture was concentrated under reduced pressure. The residue was purified by flash chromatography on silica gel column using 2%-10% of MeOH in CH₂Cl₂ as eluent to provide 46 mg of crude product. The crude product was purified by flash chromatography on pre-packed C18 column using 20-50% of MeCN in water (10 mmol/L NH₄HCO₃) as eluent to provide N-(5-(4-(1-methyl-H-pyrazol-5-yl)phenyl)-8-(methylamino)-2,7-naphthyridin-3-yl)cyclopropanecarboxamide as a white solid (23.6 mg, 21%). LCMS (ESI) m/z 399.2, [M+H]⁺. ¹H NMR (400 MHz, DMSO-d₆) δ 10.97 (s, 1H), 9.40 (s, 1H), 8.41 (s, 1H), 8.07-8.02 (m, 1H), 8.00 (s, 1H), 7.68-7.66 (m, 2H), 7.56-7.54 (m, 3H), 6.49 (d, J=1.6 Hz, 1H), 3.94 (s, 3H), 3.02 (d, J=4.4 Hz, 3H), 2.07-1.99 (m, 1H), 0.81-0.77 (m, 4H)

### Example 174 and Example 175: Synthesis of (E)-N-(5-(2-methoxystyryl)-8-(methylamino)-2, 7-naphthyridin-3-yl)cyclopropanecarboxamide and (2)-N-(5-(2-methoxystyryl)-8-(methylamino)-2,7-naphthyridin-3-yl)cyclopropanecarboxamide

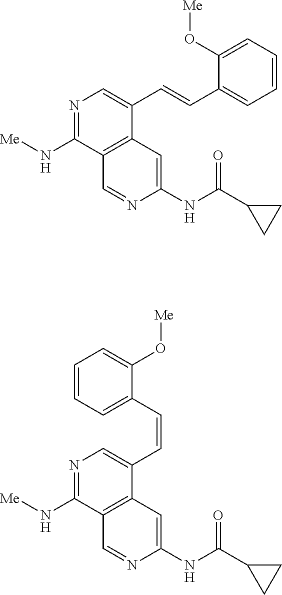 [SMILES: CNc1ncc(/C=C/c2ccccc2OC)c2cc(NC(=O)C3CC3)ncc12 ; CNc1ncc(/C=C\c2ccccc2OC)c2cc(NC(=O)C3CC3)ncc12]

To a stirring mixture of N-(5-bromo-8-(methylamino)-2,7-naphthyridin-3-yl)cyclopropanecarboxamide (60 mg; 0.187 mmol; 1.00 eq.) in DMF (2 mL) was added Pd(PPh₃)₂Cl₂ (13.1 mg; 0.019 mmol; 0.10 eq.), Na₂CO₃ (39.6 mg; 0.374 mmol; 2.00 eq.) and 1-methoxy-2-vinylbenzene (25.1 mg; 0.187 mmol; 1.00 eq.) at room temperature. The resulting mixture was stirred for 2 h at 100° C. under nitrogen atmosphere. The desired product was detected via LCMS. The mixture was allowed to cool down to room temperature. The resulting mixture was concentrated under reduced pressure. The residue was purified by flash chromatography on silica gel column using MeOH/CH₂Cl₂ (1-6%) as eluent to provide the crude desired product. The product was re-purified by reverse phase preparative HPLC (Prep-C18, gradient elution of 40-50% MeCN in water over a 10 min period, where both water and MeCN contain 10 mmol/L NH₄HCO₃, flow rate: 60 mL/min, detector UV wavelength: 254 nm) to provide (E)-N-(5-(2-methoxystyryl)-8-(methylamino)-2,7-naphthyridin-3-yl)cyclopropanecarboxamide as a yellow solid (4.2 mg, 6%) and (Z)—N-(5-(2-methoxystyryl)-8-(methylamino)-2,7-naphthyridin-3-yl)cyclopropanecarboxamide as a white solid (7.5 mg, 10%). LCMS (ESI) m/z 375.2, [M+H]⁺. HNMR for Example 174: ¹H NMR (400 MHz, DMSO-d₆) δ 11.02 (s, 1H), 9.35 (s, 1H), 8.57 (s, 1H), 8.29 (s, 1H), 8.07-7.99 (m, 1H), 7.65-7.55 (m, 1H), 7.40 (d, J=16.4 Hz, 1H), 7.32-7.17 (m, 2H), 7.08-6.96 (m, 2H), 3.86 (s, 3H), 3.00 (d, 4.4 Hz, 3H), 2.11-2.02 (m, 1H), 0.91-0.79 (m, 4H). HNMR for Example 175: ¹H NMR (400 MHz, DMSO-d₆) δ 10.78 (s, 1H), 9.29 (s, 1H), 8.26 (s, 1H), 7.88-7.81 (m, 1H), 7.77 (s, 1H), 7.27-7.20 (m, 1H), 7.10-7.06 (m, 1H), 7.01-6.95 (m, 1H), 6.90-6.80 (m, 1H), 5.64 (d, J=2.0 Hz, 1H), 5.42 (d, J=2.0 Hz, 1H), 3.63 (s, 3H), 2.97 (d, J=4.4 Hz, 3H), 2.03-1.96 (m, 1H), 0.82-0.74 (m, 4H).

### Example 176: Synthesis of N-(8-(methylamino)pyrido[3,4-c]pyridazin-3-yl)cyclopropanecarboxamide

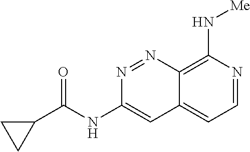 [SMILES: CNc1nccc2cc(NC(=O)C3CC3)nnc12]

### Step 1: 1-(tert-butyl) 5-ethyl (2)-2-((diphenylmethylene)amino)-3-methylpent-2-enedioate

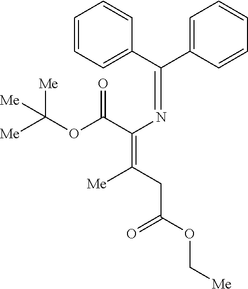 [SMILES: CCOC(=O)C/C(C)=C(\N=C(c1ccccc1)c1ccccc1)C(=O)OC(C)(C)C]

To a solution of PPh₃ (887 mg; 3.38 mmol; 0.05 eq.) in toluene (150 mL) at 0° C. was added t-BuOK (3.80 g; 33.8 mmol; 0.50 eq.) and t-BuOH (2.51 g; 33.8 mmol; 0.50 eq.). To this mixture was added a solution of tert-butyl 2-((diphenylmethylene)amino)acetate (20 g; 67.7 mmol; 1.00 eq.) in 150 mL toluene was added slowly and followed by addition of ethyl but-2-ynoate (7.59 g; 67.7 mmol; 1.00 eq.) slowly at 0° C. The reaction was stirred at 0° C. for 10 min. The reaction mixture was quenched with EtOH (150 mL). The resulting mixture was concentrated under reduced pressure. The residue was purified by flash chromatography on silica gel column using 20-60% of EtOAc in petroleum ether as eluent to provide 1-(tert-butyl) 5-ethyl (7)-2-((diphenylmethylene)amino)-3-methylpent-2-enedioate as a yellow solid (18 g, 65%). LCMS (ESI) m/z 408.2, [M+H]⁺.

### Step 2: tert-butyl 4-methyl-6-oxo-1,4,5,6-tetrahydropyridazine-3-carboxylate

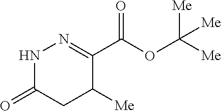 [SMILES: CC1CC(=O)NN=C1C(=O)OC(C)(C)C]

To a solution of hydrazine hydrochloride (7.05 g; 102 mmol; 3.99 eq.) in EtOH (50 mL) was added AcONa (8.45 g; 103 mmol; 4.00 eq.). To this mixture was added a solution of 1-tert-butyl) 5-ethyl (Z)-2-((diphenylmethylene)amino)-3-methylpent-2-enedioate (10.5 g; 25.7 mmol; 1.00 eq.) in EtOH (150 mL) was added slowly at 0° C. and the mixture was allowed to warm to 80° C. and stirred for 16 h. The resulting mixture was cooled to room temperature and concentrated under reduced pressure. The resulting mixture was diluted with water (200 mL) and extracted with EtOAc (3×100 mL). The combined organic layers were washed with a sat. NaCl solution (1×100 mL), dried over anhydrous Na₂SO₄. The organic layers were concentrated under reduced pressure. The residue was purified by flash chromatography on silica gel column using 20-60% of EtOAc in petroleum ether as eluent to provide tert-butyl 4-methyl-6-oxo-1,4,5,6-tetrahydropyridazine-3-carboxylate as a yellow solid (5.5 g, 58%). LCMS (ESI) m/z 213.1, [M+H]⁺.

### Step 3: 4-methyl-6-oxo-1,6-dihydropyridazine-3-carboxylic acid

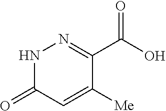 [SMILES: Cc1cc(=O)[nH]nc1C(=O)O]

To a solution of tert-butyl 4-methyl-6-oxo-1,4,5,6-tetrahydropyridazine-3-carboxylate (5 g; 23.5 mmol; 1.00 eq.) in AcOH (35 mL) was added Br₂ (7.5 g; 46.8 mmol; 2.00 eq.). The mixture was stirred for 30 min at 120° C. The desired product was observed via LCMS. The resulting mixture was added dropwise to water (100 mL) at 0° C. and stirred for 30 min at room temperature until precipitate solids were formed. The precipitated solids were collected by filtration to afford 3.5 g of the crude product as a yellow solid. The crude product was slurried in a mixed solvent of CH₂Cl₂/petroleum ether (1:1, 10 mL). After filtration, the filter cake was washed with a mixture solvent of CH₂Cl₂/petroleum ether (1:1, 4 mL). The solids were dried under reduced pressure to provide 4-methyl-6-oxo-1,6-dihydropyridazine-3-carboxylic acid as a brown solid (2.82 g, 77%). LCMS (ESI) m/z 155.0, [M+H]⁺.

### Step 4: methyl 4-methyl-6-oxo-1,6-dihydropyridazine-3-carboxylate

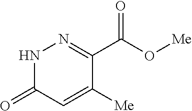 [SMILES: COC(=O)c1n[nH]c(=O)cc1C]

To a solution of 4-methyl-6-oxo-1,6-dihydropyridazine-3-carboxyli c acid (2.3 g; 14.92 mmol; 1.00 eq.) in MeOH (35 mL) was added SOCl₂ (2.13 g; 17.9 mmol, 1.20 eq.) at 0° C. The mixture was stirred for 3 h at 75° C. The desired product was observed via LCMS. Upon completion, the solids were collected by filtration and washed with MeOH (5×3 mL). The solid was dried under vacuum to provide methyl 4-methyl-6-oxo-1,6-dihydropyridazine-3-carboxylate as a yellow solid (2.5 g, 99%). LCMS (ESI) m/z 169.1, [M+H]⁺.

### Step 5: methyl 6-chloro-4-methylpyridazine-3-carboxylate

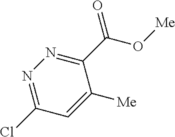 [SMILES: COC(=O)c1nnc(Cl)cc1C]

Methyl 4-methyl-6-oxo-1,6-dihydropyridazine-3-carboxylate (2.7 g; 16.0 mmol; 1.00 eq.) was dissolved in POCl₃ (20 mL). The reaction mixture was stirred for 40 min at 100° C. under nitrogen atmosphere. The desired product was detected by LCMS. The mixture was allowed to cool down to room temperature. The resulting mixture was concentrated under reduced pressure. The residue was dissolved in CH₂Cl₂ (250 mL). The mixture was basified to pH 9 with a saturated sodium bicarbonate solution. The resulting mixture was extracted with CH₂Cl₂ (2×150 mL). The combined organic layers were washed with saturated sodium chloride solution (1×50 mL), dried over anhydrous Na₂SO₄. After filtration, the filtrate was concentrated under reduced pressure. The residue was purified by flash chromatography on silica gel column using 10-40% of EtOAc in CH₂Cl₂ as eluent, to provide methyl 6-chloro-4-methylpyridazine-3-carboxylate as a white solid (2.76 g, 92%). LCMS (ESI) m/z 187.0, [M+H]⁺.

### Step 6: methyl 6-(cyclopropanecarboxamido)-4-methylpyridazine-3-carboxylate

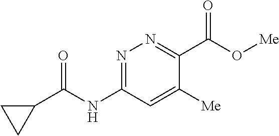 [SMILES: COC(=O)c1nnc(NC(=O)C2CC2)cc1C]

To a stirring mixture of methyl 6-chloro-4-methylpyridazine-3-carboxylate (600 mg; 3.22 mmol; 1.00 eq.) in 1,4-dioxane (20 mL) was added cyclopropanecarboxamide (1.09 g; 12.9 mmol; 4.00 eq.), Pd₂(dba)₃ (294 mg; 0.321 mmol; 0.10 eq.), XantPhos (372 mg; 0.643 mmol; 0.20 eq.) and Cs₂CO₃ (2.1 g; 6.40 mmol; 2.00 eq.). The resulting mixture was stirred for 2 h at 110° C. under nitrogen atmosphere. The desired product was observed via LCMS. The resulting mixture was concentrated under reduced pressure. The residue was purified by flash chromatography on silica gel column using 20-60% of EtOAc in petroleum ether as eluent, to provide methyl 6-(cyclopropanecarboxamido)-4-methylpyridazine-3-carboxylate as a yellow solid (320 mg, 42%). LCMS (ESI) m/z 236.1, [M+H]⁺.

### Step 7: 6-(cyclopropanecarboxamido)-4-methylpyridazine-3-carboxamide

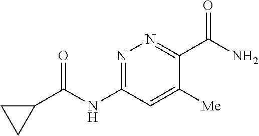 [SMILES: Cc1cc(NC(=O)C2CC2)nnc1C(N)=O]

Methyl 6-(cyclopropanecarboxamido)-4-methylpyridazine-3-carboxylate (320 mg; 1.36 mmol; 1.00 eq.) was dissolved in a solution of NH₃ in MeOH (7 M, 15 mL). The reaction mixture was stirred overnight at room temperature. The desired product was observed via LCMS. The reaction mixture was filtered through a filter paper and concentrated under reduced pressure to provide 6-(cyclopropanecarboxamido)-4-methylpyridazine-3-carboxamide as a white solid (280 mg, 93%). LCMS (ESI) m/z 221.1, [M+H]⁺.

### Step 8: (E)-6-(cyclopropanecarboxamido)-N-((dimethylamino)methylene)-4-methylpyridazine-3-carboxamide

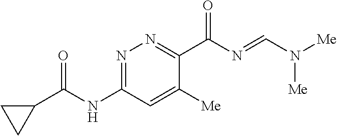 [SMILES: Cc1cc(NC(=O)C2CC2)nnc1C(=O)/N=C/N(C)C]

To a stirring solution of 6-(cyclopropanecarboxamido)-4-methylpyridazine-3-carboxamide (280 mg; 1.2.7 mmol; 1.00 eq.) in methyl tetrahydrofuran (30 mL) was added DMF-DMA (303 mg; 2.54 mmol; 2.00 eq.). The reaction mixture was stirred for 1 h at 80° C. under nitrogen atmosphere. The desired product could be detected by LCMS. The resulting mixture was concentrated under reduced pressure to provide (E)-6-(cyclopropanecarboxamido)-N-((dimethylamino)methylene)-4-methylpyridazine-3-carboxamide as a white solid (300 mg, crude). LCMS (ESI) m/z 276.1, [M+H]⁺.

### Step 9: N-(8-oxo-7,8-dihydropyrido[3,4-c]pyridazin-3-yl)cyclopropanecarboxamide

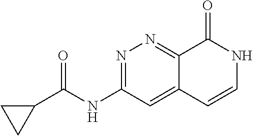 [SMILES: O=C(Nc1cc2cc[nH]c(=O)c2nn1)C1CC1]

To a stirring solution of (E)-6-(cyclopropanecarboxamido)-N-((dimethylamino)methylene)-4-methylpyridazine-3-carboxamide (300 mg; 1.09 mmol; 1.00 eq.) in THF (12 mL) was added a solution of t-BuOK in THF (1 M, 12 mL) at −40° C. and stirred for 2 h at room temperature under nitrogen atmosphere. The desired product was observed via LCMS. The resulting mixture was concentrated under reduced pressure. The mixture was diluted with water (1 mL) and acidified to pH≈6 with a solution of HCl (1 M) and solids were formed. The solids were filtered through a filter paper and dried under reduced pressure to provide N-(8-oxo-7,8-dihydropyrido[3,4-c]pyridazin-3-yl)cyclopropanecarboxamide as a yellow solid (133 mg, 95%). LCMS (ESI) m/z 231.1, [M+H]⁺.

### Step 10: N-(8-chloropyrido[3,4-c]pyridazin-3-cyclopropanecarboxamide

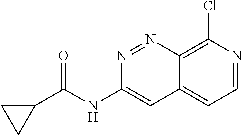 [SMILES: O=C(Nc1cc2ccnc(Cl)c2nn1)C1CC1]

N-(8-oxo-7,8-dihydropyrido[3,4-c]pyridazin-3-yl)cyclopropanecarboxamide (120 mg; 0.521 mmol; 1.00 eq.) was dissolved in POCK (5 mL). The resulting mixture was stirred for 2 h at 100° C. under nitrogen atmosphere. The desired product could be detected by LCMS. The mixture was allowed to cool down to room temperature. The resulting mixture was concentrated under reduced pressure. The residue was dissolved in CH₂Cl₂, (40 mL). The mixture was basified to pH≈9 with a saturated sodium bicarbonate solution. The resulting mixture was extracted with CH₂Cl₂ (3×50 mL). The combined organic layers were washed with saturated sodium chloride solution (1×10 mL), dried over anhydrous Na₂SO₄. After filtration, the filtrate was concentrated under reduced pressure to provide N-(8-chloropyrido[3,4-c]pyridazin-3-yl)cyclopropanecarboxamide as a black solid (75 mg, crude). LCMS (ESI) ink 249.0, [M+H]⁺.

### Step 11: N-(8-(methylamino)pyrido[3,4-c]pyridazin-3-yl)cyclopropanecarboxamide

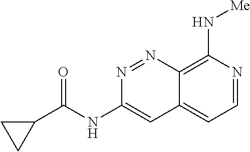 [SMILES: CNc1nccc2cc(NC(=O)C3CC3)nnc12]

To a stirring solution of N-(8-chloropyrido[3,4-c]pyridazin-3-yl)cyclopropanecarboxamide (75 mg; 0.302 mmol; 1.00 eq.) in NMP (3 mL) was added methanamine hydrochloride (60.7 mg; 0.905 mmol; 3.01 eq.) and DIPEA (195 mg; 1.51 mmol; 5.00 eq.). The reaction mixture was stirred for 1.5 h at 100° C. under nitrogen atmosphere. The desired product could be detected via LCMS. The reaction mixture was purified by flash chromatography on pre-packed C18 column using 20-70% of MeOH in water (10 mmol/L NH₄HCO₃) to provide the desired product as a yellow solid (45 mg, 61%). 10 mg sample was further purified by flash chromatography on pre-packed C18 column using 10-50% of MeCN in water (10 mmol/L NH₄HCO₃) to provide N-(8-(methylamino)pyrido[3,4-c]pyridazin-3-yl)cyclopropanecarboxamide as a yellow solid (1.4 mg, 14%). LCMS (ESI) m/z 244.1, [M+H]⁺. ¹H NMR (400 MHz, DMSO-d₆) δ 11.65 (s, 1H), 8.55-8.45 (m, 1H), 8.42 (s, 1H), 7.98 (d, J=5.6 Hz, 1H), 6.76 (d, J=5.6 Hz, 1H), 3.03 (d, J=4.4 Hz, 3H), 2.19-2.13 (m, 1H), 0.90-0.88 (m, 4H).

### Example 177: Synthesis of N-(5-bromo-8-(methylamino)pyrido[3,4-c]pyridazin-3-yl)cyclopropanecarboxamide

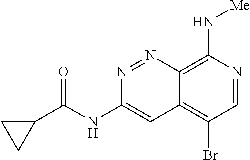 [SMILES: CNc1ncc(Br)c2cc(NC(=O)C3CC3)nnc12]

To a stirring solution of N-(8-(methylamino)pyrido[3,4-c]pyridazin-3-yl)cyclopropanecarboxamide (32 mg; 0.132 mmol; 1.00 eq.) in DMF (2 mL) was added N-bromosuccinimide (23.4 mg; 0.131 mmol; 1.00 eq.) at 0° C. and stirred for 1 h at room temperature. The mixture was diluted with EtOAc (50 mL) and washed with a saturated sodium chloride solution (2×5 mL). The organic layer was dried over anhydrous Na₂SO₄. After filtration, the filtrate was concentrated under reduced pressure. The crude product was purified by flash chromatography on pre-packed C18 column using 20-60% of MeCN in water (10 mmol/L to provide N-(5-bromo-8-(methylamino)pyrido[3,4-c]pyridazin-3-yl)cyclopropanecarboxamide as a white solid (35.5 mg, 83%); LCMS (ESI) m/z 322.0, [M+H]⁺. ¹H NMR (400 MHz, DMSO-d₆) δ 11.86 (s, 8.82-8.79 (m, 8.58 (s, 1H), 8.20 (s, 1H), 3.02 (d, J=4.4 Hz, 3H), 2.21-2.08 (m, 1H), 0.97-0.83 (m, 4H).

### Example 178: Synthesis of N-(8-(methylamino)-5-phenylpyrido[3,4-c]pyridazin-3-yl)cyclopropanecarboxamide

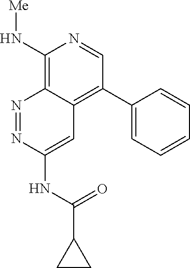 [SMILES: CNc1ncc(-c2ccccc2)c2cc(NC(=O)C3CC3)nnc12]

To a stirring solution of N-(5-bromo-8-(methylamino)pyrido[3,4-c]pyridazin-3-yl)cyclopropanecarboxamide (21.2 mg; 0.066 mmol; 1.00 eq.) in DME/water (5:1, 2.4 mL) was added phenylboronic acid (24.1 mg; 0.198 mmol; 3.00 eq.), Pd(PPh₃)₄ (70.6 mg; 0.007 mmol; 0.10 eq.) and Na₂CO₃ (21.0 mg; 0.198 mmol; 3.00 eq.). The resulting mixture was stirred for 1 h at 100° C. The resulting mixture was cooled to room temperature and concentrated under reduced pressure. The residue was purified by flash chromatography on silica gel column using 0-15% of MeOH in CH₂Cl₂ as eluent to provide N-(8-(methylamino)-5-phenylpyrido[3,4-c]pyridazin-3-yl)cyclopropanecarboxamide as a yellow solid (39.4 mg, crude). The crude product was further purified by flash chromatography on pre-packed C18 column using 20-50% of MeCN in water (10 mmol/L NH₄HCO₃) provide N-(8-(methylamino)-5-phenylpyrido[3,4-c]pyridazin-3-yl)cyclopropanecarboxamide as a white solid (13.8 mg, 65%). LCMS (ESE) m/z 320.1, [M+H]⁺. ¹H NMR (400 MHz, DMSO-d₆) δ 11.69 (s, 1H), 8.71-8.61 (m, 1H), 8.56 (s, 1H), 8.00 (s, 1H), 7.57-7.38 (m, 5H), 3.09 (d, J=4.4 Hz, 3H), 2.19-2.07 (m, 1H), 0.97-0.83 (m, 41).

### Examples 179-183

Each compound in Table 8 below was prepared following the same experimental procedure as previously described in Example 155 using N-(8-(methylamino)-5 (4,4,5,5-tetramethyl-1,3,2-dioxaborolan-2-yl)-2,7-naphthyridin-3-yl)cyclopropanecarboxamide as the common intermediate and appropriate halogenated aromatic starting material.

TABLE 8

| Example | Structure and Name | MS and ¹H NMR |
|---|---|---|
| Example 179 | 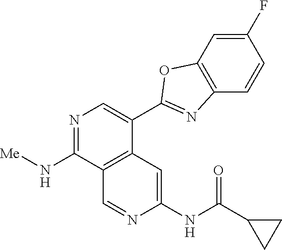 [SMILES: CNc1ncc(-c2nc3ccc(F)cc3o2)c2cc(NC(=O)C3CC3)ncc12] | LCMS (ESI) m/z 378.1, [M + H]⁺. ¹H NMR (400 MHz, DMSO-d₆) δ 11.04 (s, 1H), 9.64 (s, 1H), 9.43 (s, 1H), 8.87 (s, 1H), 8.71-8.63 (m, 1H), 7.81-7.75 (m, 2H), 7.29-7.19 (m, 1H), 3.09 (d, J = 4.4 Hz, 3H), 2.15-2.04 (m, 1H), 0.95-0.81 (m, 4H). |
| | N-(5-(6-fluorobenzo[d]oxazol- | |
| | 2-yl)-8-(methylamino)-2,7- | |
| | naphthyridin-3- | |
| | yl)cyclopropanecarboxamide | |
| Example 180 | 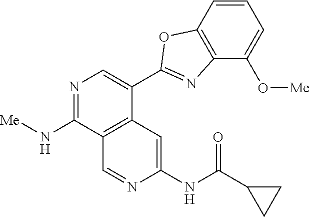 [SMILES: CNc1ncc(-c2nc3c(OC)cccc3o2)c2cc(NC(=O)C3CC3)ncc12] | LCMS (ESI) m/z 390.1, [M + H]⁺. ¹H NMR (400 MHz, DMSO-d₆) δ 11.04 (s, 1H), 9.65 (s, 1H), 9.44 (s, 1H), 8.88 (s, 1H), 8.66-8.58 (m, 1H), 7.33-7.26 (m, 2H), 6.92 (dd, J = 8.4, 3.6 Hz, 1H), 4.17 (s, 3H), 3.09 (d, J = 4.4 Hz, 3H), 2.13- 2.02 (m, 1H), 0.90-0.80 (m, 4H). |
| | N-(5-(4- | |
| | methoxybenzo[d]oxazol-2-yl)- | |
| | 8-(methylamino)-2,7- | |
| | naphthyridin-3- | |
| | yl)cyclopropanecarboxamide | |
| Example 181 | 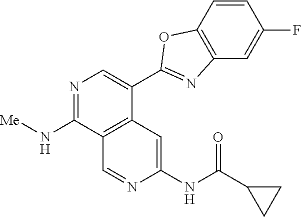 [SMILES: CNc1ncc(-c2nc3cc(F)ccc3o2)c2cc(NC(=O)C3CC3)ncc12] | LCMS (ESI) m/z 378.1, [M + H]⁺. ¹H NMR (400 MHz, DMSO-d₆) δ 11.04 (s, 1H), 9.62 (s, 1H), 9.44 (s, 1H), 8.91 (s, 1H), 8.74-8.66 (m, 1H), 7.80-7.72 (m, 1H), 7.64-7.57 (m, 1H), 7.28-7.19 (m, 1H), 3.09 (d, J = 4.4 Hz, 3H), 2.16-2.05 (m, 1H), 0.95-0.76 (m, 4H). |
| | N-(5-(5-fluorobenzo[d]oxazol- | |
| | 2-yl)-8-(methylamino)-2,7- | |
| | naphthyridin-3- | |
| | yl)cyclopropanecarboxamide | |
| Example 182 | 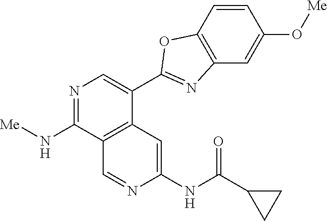 [SMILES: CNc1ncc(-c2nc3cc(OC)ccc3o2)c2cc(NC(=O)C3CC3)ncc12] | LCMS (ESI) m/z 390.1, [M + H]⁺. ¹H NMR (400 MHz, DMSO-d₆) δ 11.02 (s, 1H), 9.64 (s, 1H), 9.44 (s, 1H), 8.90 (s, 1H), 8.66-8.60 (m, 1H), 7.62 (d, J = 8.8 Hz, 1H), 7.28 (d, J = 2.4 Hz, 1H), 6.96 (dd, J = 8.8, 2.4 Hz, 1H), 3.85 (s, 3H), 3.09 (d, J = 4.4 Hz, 3H), 2.14-2.06 (m, 1H), 0.94- 0.82 (m, 4H). |
| | N-(5-(5- | |
| | methoxybenzo[d]oxazol-2-yl)- | |
| | 8-(methylamino)-2,7- | |
| | naphthyridin-3- | |
| | yl)cyclopropanecarboxamide | |
| Example 183 | 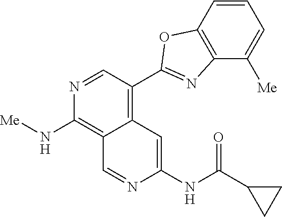 [SMILES: CNc1ncc(-c2nc3c(C)cccc3o2)c2cc(NC(=O)C3CC3)ncc12] | LCMS (ESI) m/z 374.2, [M + H]⁺. ¹H NMR (400 MHz, DMSO-d₆) δ 11.01 (s, 1H), 9.90 (s, 1H), 9.43 (s, 1H), 8.90 (s, 1H), 8.64-8.60 (m, 1H), 7.55- 7.49 (m, 1H), 7.30-7.16 (m, 2H), 3.09 (d, J = 4.4 Hz, 3H), 2.63 (s, 3H), 2.16- 2.05 (m, 1H), 0.95-0.78 (m, 4H). |
| | N-(8-(methylamino)-5-(4- | |
| | methylbenzo[d]oxazol-2-yl)- | |
| | 2,7-naphthyridin-3- | |
| | yl)cyclopropanecarboxamide | |

### Example 184: Synthesis of N-(5-(4-fluorobenzo(dioxazol-2-yl)-8-(methylamino)-2,7-naphthyridin-3-yl)cyclopropanecarboxamide

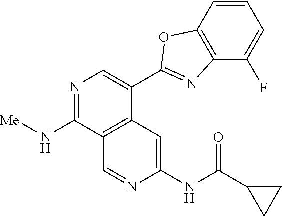 [SMILES: CNc1ncc(-c2nc3c(F)cccc3o2)c2cc(NC(=O)C3CC3)ncc12]

### Step 1: 4-fluorobenzo[d]oxazole-2(3H)-thione

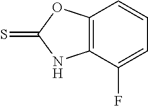 [SMILES: Fc1cccc2oc(=S)[nH]c12]

A stirring mixture of 2-amino-3-fluorophenol (1.0 g; 7.86 mmol; 1.0 eq.) and potassium O-ethyl carbonodithioate (1.39 g; 8.65 mmol; 1.1 eq.) in MeOH (20 mL) was stirred for 3 h at 70° C. under nitrogen atmosphere. The desired product was observed via LCMS. Upon completion; the resulting mixture was concentrated under vacuum. The residue was dissolved in water (20 mL). The mixture was acidified to pH=4 with conc. HCL. The solids were collected by filtration and washed with water (10 mL) to afford 4-fluorobenzo[d]oxazole-2(3H)-thione as a grey solid (500 mg, 37%). LCMS (ESI) m/z 170.0, [M+H]⁺.

### Step 2: 2-chloro-4-fluorobenzo[d]oxazole

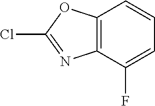 [SMILES: Fc1cccc2oc(Cl)nc12]

To a mixture of 4-fluorobenzo[d]oxazole-2(3H)-thione (500.0 mg; 2.95 mmol; 1.0 eq.) in SOCl₂ (3.2 mL; 44.3 mmol; 15.0 eq.) was added dropwise DMF (0.06 mL; 0.775 mmol; 0.26 eq.) at 0° C. The resulting mixture was stirred for 2.5 h at room temperature. The desired product was observed via LCMS. Upon completion, the resulting mixture was concentrated under reduced pressure. The residue was purified by flash chromatography on silica gel column using EtOAc in petroleum ether (25-60%) as eluent to provide 2-chloro-4-fluorobenzo[d]oxazole as a yellow solid (210 mg, 41%). LCMS (ESI) m/z 172.0, [M+H]⁺.

### Step 3: N-(5-(4-fluorobenzo[d]oxazol-2-yl)-8-(methylamino)-2,7-naphthyridin-3-yl)cyclopropanecarboxamide

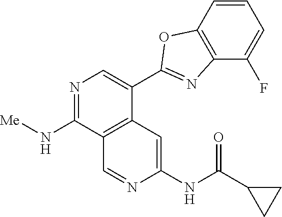 [SMILES: CNc1ncc(-c2nc3c(F)cccc3o2)c2cc(NC(=O)C3CC3)ncc12]

N-(5-(4-fluorobenzo[d]oxazol-2-yl)-8-(methylamino)-2,7-naphthyridin-3-yl)cyclopropanecarboxamide was synthesized using the same procedure that was previously described in Example 155 using N-(8-(methylamino)-5-(4,4,5,5-tetramethyl-1,3,2-dioxaborolan-2-yl)-2,7-naphthyridin-3-yl)cyclopropanecarboxamide and 2-chloro-4-fluorobenzo[d]oxazole as the starting materials; LCMS (ESI) m/z 378.1, [M+H]⁺. ¹H NMR (300 MHz, DMSO-d₆) δ 11.05 (s, 1H), 9.65 (s, 1H), 9.45 (s, 1H), 8.93 (s, 1H), 8.78-8.67 (m, 1H), 7.68-7.56 (m, 1H), 7.47-7.35 (m, 1H), 7.32-7.22 (m, 1H), 3.10 (d, J=4.4 Hz, 31-1), 2.16-2.03 (m, 1H), 0.99-0.82 (m, 4H).

### Example 185: Synthesis of N-(5-(7-fluorobenzo[d]oxazol-2-yl)-68-(methylamino)-2,7-naphthyridin-3-yl)cyclopropanecarboxamide

### Step

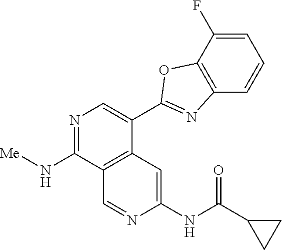 [SMILES: CNc1ncc(-c2nc3cccc(F)c3o2)c2cc(NC(=O)C3CC3)ncc12]

Step 1: N-(5-(hydroxymethyl)-8-(methylamino)-2,7-naphthyridin-3-yl)cyclopropanecarboxamide

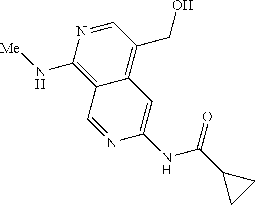 [SMILES: CNc1ncc(CO)c2cc(NC(=O)C3CC3)ncc12]

A mixture of N-(5-bromo-8-(methylamino)-2,7-naphthyridin-3-yl)cyclopropanecarboxamide (3.0 g; 9.37 mmol; 1.00 eq.), (tributylstannyl)methanol (9.05 g; 28.1 mmol; 3.00 eq.), Pd(PPh₃)₄ (1.08 g; 0.937 mmol; 0.10 eq.) in 1,4-dioxane (10 mL) was reacted overnight at 90° C. under N₂ atmosphere. The mixture was cooled to room temperature and concentrated under reduced pressure. The crude product mixture was purified by via silica gel column using 0-20% of MeOH in CH₂Cl₂ as eluent to provide N-(5-(hydroxymethyl)-8-(methylamino)-2,7-naphthyridin-3-yl)cyclopropanecarboxamide as a yellow solid (1.4 g, 54%). LCMS (ESI) m/z 273.1, [M+H]⁺.

### Step 2: N-(5-formyl-8-(methylamino)-2,7-naphthyridin-3-yl)cyclopropanecarboxamide

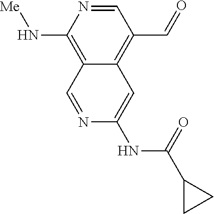 [SMILES: CNc1ncc(C=O)c2cc(NC(=O)C3CC3)ncc12]

Under N₂ atmosphere, to a mixture of N-(5-(hydroxymethyl)-8-(methylamino)-2,7-naphthyridin-3-yl)cyclopropanecarboxamide (1.12 g; 4,113 mmol; 1.00 eq.) in CH₂Cl₂ (150 mL) were added NMO (1.02 g; 8.72 mmol; 2.12 eq.) and TPAP (289 mg; 0.823 mmol; 0.20 eq.). The reaction was stirred for 2 h at 25° C., then another TPAP (289 mg; 0.823 mmol; 0.20 eq.) was added and stirred for another 2 h at 25° C. Upon completion, the mixture was concentration under reduced pressure. The residue was purified by flash chromatography on silica gel column using 0-20% of MeOH in CH₂Cl₂ as eluent to provide N-(5-formyl-8-(methylamino)-2,7-naphthyridin-3-yl)cyclopropanecarboxamide as a yellow solid (300 mg, 27%). LCMS (ESI) m/z 271.1, [M+H]⁺.

### Step 3: N-(5-(7-fluorobenzo[d]oxazol-2-yl)-8-(methylamino)-2,7-naphthyridin-3-yl)cyclopropanecarboxamide

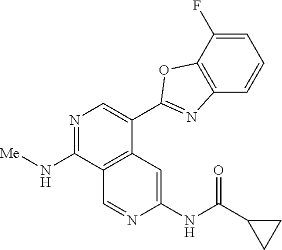 [SMILES: CNc1ncc(-c2nc3cccc(F)c3o2)c2cc(NC(=O)C3CC3)ncc12]

A mixture of N-(5-formyl-8-(methylamino)-2,7-naphthyridin-3-yl)cyclopropanecarboxamide (20.0 mg; 0.074 mmol; 1.00 eq.) and 2-amino-6-fluorophenol (9.4 mg; 0.0740 mmol; 1.00 eq.) in toluene (3 mil-) was reacted overnight at 110° C. under-N₂ atmosphere. The mixture was concentrated under reduced pressure. The residue was dissolved in CH₂Cl₂ (3 mL) and DDQ (18.5 mg; 0.0810 mmol; 1.10 eq.) was added under N₂ and the mixture reaction was stirred for 2 h at room temperature. Upon completion, the mixture was concentrated under reduced pressure. The residue was purified by flash chromatography on pre-packed C18 column using 5-100% of MeCN in water (10 mmol/L NH₄HCO₃) as eluent to afford N-(5-(7-fluorobenzo[d]oxazol-2-yl)-8-(methylamino)-2,7-naphthyridin-3-yl)cyclopropanecarboxamide as a grey solid (2.6 mg, 9%). LCMS (ESI) m/z 378.1, [M+H]⁺. ¹H NMR (400 MHz, DMSO-d₆) δ 11.05 (s, 1H), 9.65 (s, 1H), 9.44 (s, 1H), 8.94 (s, 1H), 8.75-8.71 (m, 1H), 7.65-7.60 (m, 1H), 7.45-7.23 (m, 2H), 3.10 (d, J=4.4 Hz, 3H), 2.16-2.02 (m, 1H), 0.94-0.82 (m, 4H).

### Example 186: Synthesis of N-(8-(methylamino)-5-(oxazolo[5,4-b]pyridin-2-yl)-2,7-naphthyridin-3-yl)cyclopropanecarboxamide

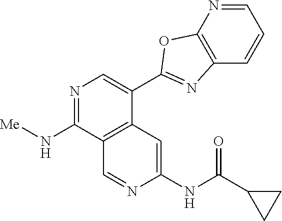 [SMILES: CNc1ncc(-c2nc3cccnc3o2)c2cc(NC(=O)C3CC3)ncc12]

### Step 1: 2-iodooxazolo[5,4-b]pyridine

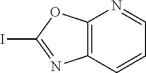

A solution of oxazolo[5,4-b]pyridin-2-amine (300 mg; 2.220 mmol; 1.00 eq.) in acetone (6 mL) was treated with NaI (682.2 mg; 4551 mmol; 2.05 eq.) at room temperature under air atmosphere, followed by the addition of t-BuONO (8.013 g; 77.70 mmol; 35.00 eq.) dropwise at room temperature. The resulting mixture was stirred for 3 days at room temperature. The desired product was observed via LCMS. Upon completion, the reaction mixture was concentrated under reduced pressure. The residue was purified via a silica gel column using 10-30% of EtOAc in petroleum ether as eluent to provide 2-iodooxazolo[5,4-b]pyridine as a colorless oil (320 mg, 58%). LCMS (ESt) m/z 246.9, [M+H]⁺.

### Step 2: N-(8-(methylamino)-5-(oxazolo[5,4-b]pyridin-2-yl)-2,7-naphthyridin-3-yl)cyclopropanecarboxamide

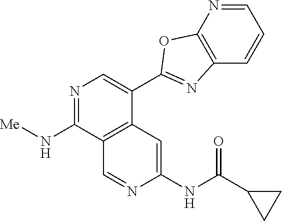 [SMILES: CNc1ncc(-c2nc3cccnc3o2)c2cc(NC(=O)C3CC3)ncc12]

N-(8-(methylamino)-5-(oxazolo[5,4-b]pyridin-2-yl)-2,7-naphthyridin-3-yl)cyclopropanecarboxamide was synthesized using similar procedure that was described in Example 155 using N-(8(methylamino)-5-(4,4,5,5-tetramethyl-1,3,2-dioxaborolan-2-yl)-2,7-naphthyridin-3-yl)cyclopropanecarboxamide and 2-iodooxazolo[5,4-b]pyridine as the starting materials. Data analysis for N-(8-(methylamino)-5-(oxazolo[5,4-b]pyridin-2-yl)-2,7-naphthyridin-3-yl)cyclopropanecarboxamide: LCMS (ESI) m/z 361.1, [M+H]⁺. ¹H NMR (400 MHz, DMSO-d₆) δ 11.06 (s, 1H), 9.68 (s, 1H), 9.45 (s, 1H), 8.97 (s, 1H), 8.79-8.72 (m, 1H), 8.32 (d, 4.4 Hz, 1H), 8.19 (d, J=7.6 Hz, 1H), 7.46 (dd, J=7.6, 4.4 Hz, 1H), 3.10 (d, J=4.4 Hz, 3H), 2.14-2.05 (m, 1H), 0.95-0.79 (m, 4H).

### Example 187: Synthesis of N-(5-(4-fluorobenzo[d]oxazol-2-yl)-8-((methyl-d₃)amino-2,7-naphthyridin-3-yl)cyclopropanecarboxamide

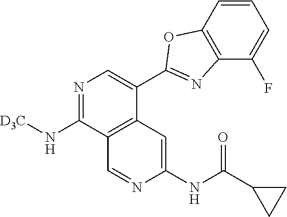 [SMILES: [2H]C([2H])([2H])Nc1ncc(-c2nc3c(F)cccc3o2)c2cc(NC(=O)C3CC3)ncc12]

### Step 1: 6-chloro-N-(methyl-d₃)-2,7-naphthyridin-1-amine

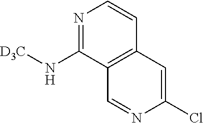 [SMILES: [2H]C([2H])([2H])Nc1nccc2cc(Cl)ncc12]

Under nitrogen atmosphere, 6-chloro-2,7-naphthyridin-1(2H)-one (4 g; 22.2 mmol; 1.00 eq.) was dissolved in DMA (100 mL). To the above mixture were added methan-d₃-amine hydrochloride (4.69 g; 66.5 mmol; 3 eq.), PyBOP (22.8 g; 43.8 mmol; 1.98 eq.) and DIPEA (14.4 g; 111 mmol; 5.03 eq.). The reaction was stirred at 80° C. overnight. The resulting mixture was cooled to rt and diluted with EtOAc (300 mL) and washed with brine (30 mL×5). The organic layer was dried with Na₂SO₄, filtered and concentrated in vacuo. The residue was purified by flash chromatography on pre-packed C18 column using 10-30% of MeCN in water (10 mmol/L NH₄HCO₃) as eluent to afford the crude product. The product was re-purified by flash chromatography on silica gel column using EtOAc in petroleum ether (10-36%) of as eluent to afford 6-chloro-N-(methyl-d₃)-2,7-naphthyridin-1-amine as a white solid (2.8 g, 64%). LCMS (ESI) m/z 197.1, [M+H]⁺.

### Step 2: N-(8-((methyl-d₃)amino)-2,7-naphthyridin-3-yl)cyclopropanecarboxamide

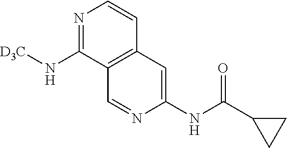 [SMILES: [2H]C([2H])([2H])Nc1nccc2cc(NC(=O)C3CC3)ncc12]

Under nitrogen atmosphere, 6-chloro-N-(methyl-d₃)-2,7-naphthyridin-1-amine (2.8 g; 14.2 mmol; 1.00 eq.) was dissolved in 1,4-dioxane (20 mL). To the above mixture was added cyclopropanecarboxamide (1.83 g; 21.4 mmol; 1.51 eq.), Pd₂(dba)₃ (10.3 g; 1.42 mmol; 0.10 eq.), XantPhos (1.64 g; 2.84 mmol; 0.20 eq.) and Cs₂CO₃ (9.3 g; 28.6 mmol; 2.02 eq.). The reaction was stirred at 110° C. overnight. The desired product was detected on LCMS. The reaction mixture was cooled to rt and concentrated under reduced pressure. The residue was purified by flash chromatography on silica gel column using MeOH in CH₂Cl₂ (10-36%) as eluent to afford the of crude product. The product was re-purified by flash chromatography on pre-packed C18 column using 10-30% of MeCN in water (10 mmol/L NH₄HCO₃) as eluent to afford N-(8-((methyl-d₃)amino)-2,7-naphthyridin-3-yl)cyclopropanecarboxamide as a off-white solid (3.26 g, 93%). LCMS (ESI) m/z 246.1, [M+H]⁺.

### Step 3: N-(5-bromo-8-((methyl-d₃)amino)-2,7-naphthyridin-3-yl)cyclopropanecarboxamide

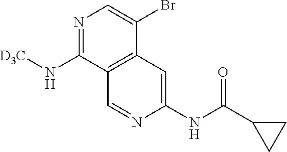 [SMILES: [2H]C([2H])([2H])Nc1ncc(Br)c2cc(NC(=O)C3CC3)ncc12]

Under nitrogen atmosphere, N-(8-((methyl-d₃)amino)-2,7-naphthyridin-3-yl)cyclopropane-carboxamide (3.26 g; 13.3 mmol; 1.00 eq.) was dissolved in DMF (25 mL) at 0° C., To the above mixture was added NBS (2.36 g; 13.3 mmol; 1.00 eq.) in portions. The reaction was then stirred at morn temperature for 1 h. The resulting mixture was diluted with EtOAc (300 mL) and washed with brine (50 mL×5). The organic layer was dried, concentrated under vacuum and purified by flash chromatography on silica gel column using MeOH in CH₂Cl₂ (2-7%) as eluent to give N-(5-bromo-8-((methyl-d₃)amino)-2,7-naphthyridin-3-yl)cyclopropanecarboxamide as a off-white solid (3.5 g, 81%). LCMS (ESI) m/z 324.0, [M+H]⁺. ¹H NMR (400 MHz, DMSO-d₆) δ 11.12 (s, 1H), 9.30 (s, 1H), 8.45 (s, 1H), 8.17 (s, 1H), 8.09-8.06 (m, 1H), 2.13-2.02 (m, 1H), 0.94-0.79 (m, 4H).

### Step 4: N-(8-((methyl-d₃)amino)-5-(4,4,5,5-tetramethyl-1,3,2-dioxaborolan-2-yl)-2,7-naphthyridin-3-yl)cyclopropanecarboxamide

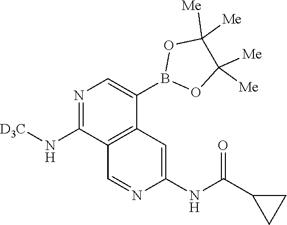 [SMILES: [2H]C([2H])([2H])Nc1ncc(B2OC(C)(C)C(C)(C)O2)c2cc(NC(=O)C3CC3)ncc12]

Under nitrogen atmosphere, N-(5-bromo-8-((methyl-d₃)am no)-2,7-naphthyridin-3-yl)cyclopropanecarboxamide (1 g; 3.08 mmol; 1 eq.) was dissolved in 1,4-dioxane (50 mL). To the above mixture was added 4,4,4′,4′,5,5,5′,5′-octamethyl-2,2′-bi(1,3,2-dioxaborolane) (7.86 g; 30.9 mmol; 10.0 eq.), Pd(dppf)Cl₂. CH₂Cl₂ (252 mg; 0.309 mmol, 0.10 eq.) and KOAc (606 mg; 6.17 mmol; 2.00 eq.). The reaction mixture was then stirred at 100° C. for 3 h. The resulting mixture was concentrated under reduced pressure. The residue was slurrying in a mixed solvent of petroleum ether/EtOAc (10:1, 100 mL) to precipitate solids. The solids were collected by filtration and washed with a mixed solvent of petroleum ether/EtOAc (10:1, 20 mL). The solids were dried under reduced pressure to afford N-(8-((methyl-d₃)amino)-5-(4,4,5,5-tetramethyl-1,3,2-dioxaborolan-2-yl)-2,7-naphthyridin-3-yl)cyclopropanecarboxamide as a brown solid (2.5 g, crude). LCMS (ESI) m/z 372.2, [M+H]⁺.

### Step 5: N-(5-(4-fluorobenzo[d]oxazol-2-yl)-8-((methyl-d₃)amino)-2,7-naphthyridin-3-yl)cyclopropanecarboxamide

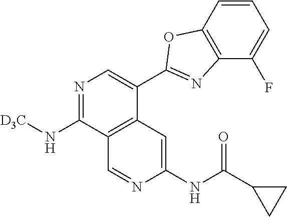 [SMILES: [2H]C([2H])([2H])Nc1ncc(-c2nc3c(F)cccc3o2)c2cc(NC(=O)C3CC3)ncc12]

N-(5-(4-fluorobenzo[d]oxazol-2-yl)-8-((methyl-d₃)amino)-2,7-naphthyridin-3-yl)cyclopropanecarboxamide was synthesized using a similar procedure that was previously described in Example 155 using N-(8-((methyl-d₃)amino)-5-(4,4,5,5-tetramethyl-1,3,2-dioxaborolan-2-yl)-2,7-naphthyridin-3-yl)cyclopropanecarboxamide and 2-chloro-4-fluorobenzo[d]oxazole as the starting materials. Analytical data for N-(5-(4-fluorobenzo[d]oxazol-2-yl)-8-((methyl-d₃)amino)-2,7-naphthyridin-3-yl)cyclopropanecarboxamide: LCMS (ESI) m/z 381.1, ¹H NMR (400 MHz, DMSO-d₆) δ 11.05 (s, 1H), 9.65 (s, 1H), 9.45 (s, 1H), 8.94 (s, 1H), 8.70 (s, 1H), 7.63-7.59 (m, 1H), 7.45-7.37 (m, 1H), 7.30-7.23 (m, 1H), 2.14-2.05 (m, 1H), 0.93-0.81 (m, 4H).

### Example 188: Synthesis of N-(5-(7-(azetidin-1-yl)-[1,2,4]triazolo[1,5-a]pyridin)-8-(methylamino)-2,7-naphthyridin-3-yl)cyclopropanecarboxamide

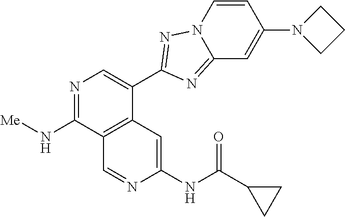 [SMILES: CNc1ncc(-c2nc3cc(N4CCC4)ccn3n2)c2cc(NC(=O)C3CC3)ncc12]

### Step 1: 7-bromo-2-chloro-[1,2,4]triazolo[1,5-a]pyridine

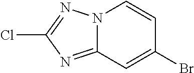 [SMILES: Clc1nc2cc(Br)ccn2n1]

A solution of 7-bromo-[1,2,4]triazolo[1,5-a]pyridin-2-amine (960 mg, 4.506 mmol; 1.00 eq.) and CuCl₂ (200 mg; 1.488 mmol, 0.33 eq.) in 12 M of hydrochloric acid (10 mL) was stirred at 5° C. for 5 min, followed by the addition of NaNO₂ (370 mg; 5.363 mmol; 1.19 eq.) in water (2 mL) was added and stirred at 5° C. for 0.5 h. Then this reaction was stirred at room temperature for 12 h. The desired product could be detected by LCMS. The reaction was added to water (30 mL) to precipitate solids. After filtration, the solid was washed with water to provide 7-bromo-2-chloro-[1,2,4]triazolo[1,5-a]pyridine as a white solid (900 mg, 85%). LCMS (ESI) m/z 231.9 [M+H]⁺.

### Step 2: 7-(azetidin-1-yl)-2-chloro-[1,2,4]triazolo[1,5-a]pyridine

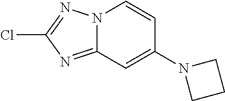 [SMILES: Clc1nc2cc(N3CCC3)ccn2n1]

Under nitrogen, a solution of 7-bromo-2-chloro-[1,2,4]triazolo[1,5-a]pyridine (400 mg; 1.721 mmol; 1.00 eq.) and Pd-PEPPSI-IHeptCl 3-chloropyridine (170 mg; 0.175 mmol; 0.10 eq.) in 1,4-dioxane (12 mL) was added azetidine (100 mg; 1.751 mmol; 1.02 eq.) and Cs₂CO₃ (1.12 g; 3.437 mmol; 2.00 eq.). The resulting solution was stirred for 1 h at 90° C. The desired product could be detected by LCMS. The reaction was cooled to room temperature and concentrated under vacuum. The residue was purified by flash chromatography on silica gel column using 20-30% of EtOAc in petroleum ether as eluent to provide 7-(azetidin-1-yl)-2-chloro-[1,2,4]triazolo[1,5-a]pyridine as a white solid (200 mg, 55%). LCMS (ESI) m/z 209.1, [M+H]⁺.

### Step 3: N-(5-(7-(azetidin-1-yl)-[1,2,4]triazolo[1,5-a]pyridin-2-yl)-8-(methylamino)-2,7-naphthyridin-3-yl)cyclopropanecarboxamide

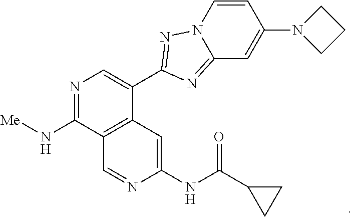 [SMILES: CNc1ncc(-c2nc3cc(N4CCC4)ccn3n2)c2cc(NC(=O)C3CC3)ncc12]

Under nitrogen, a mixture of XPhos Pd (115 mg; 0.136 mmol; 0.20 eq.), XPhos (65 mg; 0.136 mmol; 0.20 eq.) and K₃PO₄ (432 mg; 2.035 mmol; 3.00 eq.) in a mixture solvent of 1,4-dioxane/water (5:1, 9.6 mL) was added 7-(azetidin-1-yl)-2-chloro-[1,2,4]triazolo[1,5-a]pyridine (141 mg; 0.676 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (250 mg; 0.679 mmol; 1.00 eq.). The resulting solution was stirred for 1 h at 90° C. The desired product could be detected by LCMS. The reaction mixture was concentrated under vacuum. The residue was purified by flash chromatography on silica gel column using MeOH in CH₂Cl₂(10-17%) as eluent to afford the crude product. The crude product was re-purified by flash chromatography on pre-packed C18 column using 40-60% of MeCN in water (10 mmol/L NH₄HCO₃) to provide N-(5-(7-(azetidin-1-yl)-[1,2,4]triazolo[1,5-a]pyridin-2-yl)-8-(methylamino)-2,7-naphthyridin-3-yl)cyclopropanecarboxamide as a white solid (20.3 mg, 7%). LCMS (ESI) m/z 415.2, [M+H]⁺. ¹H NMR (400 MHz, DMSO-d₆) δ 10.89 (s, 1H), 9.47 (5, 1H), 9.38 (s, 1H), 8.75 (s, 1H), 8.58 (d, 7.2 Hz, 1H), 8.24-8.17 (m, 1H), 6.40 (dd, J=7.2, 2.4 Hz, 1H), 6.35 (d, J=2.4 Hz, 1H), 4.00 (t, J===7.6 Hz, 4H), 3.04 (d, J=4.4 Hz, 3H), 2.42-2.34 (m, 2H), 2.11-2.02 (m, 1H), 0.91-0.79 (m, 4H).

### Example 189: Synthesis of N-(5-(7-(3,3-difluoroazetidin-1-yl)-[1,2,4]triazolo[1,5-a]pyridin-2-yl)-8-(methylamino)-2,7-naphthyridin-3-yl)cyclopropanecarboxamide

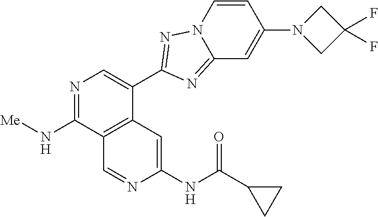 [SMILES: CNc1ncc(-c2nc3cc(N4CC(F)(F)C4)ccn3n2)c2cc(NC(=O)C3CC3)ncc12]

### Step 1: 2-chloro-7-(3,3-difluoroazetidin-1-yl)-[1,2,4]triazolo[1,5-a]pyridine

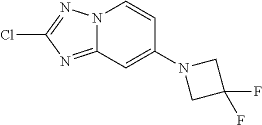 [SMILES: FC1(F)CN(c2ccn3nc(Cl)nc3c2)C1]

A mixture of 7-bromo-2-chloro-[1,2,4]triazolo[1,5-a]pyridine (Example 10, step 1) (100 mg; 0.430 mmol; 1.00 eq.), 3,3-difluoroazetidine hydrochloride (55.7 mg; 0.430 mmol; 1.00 eq.), Pd-PEPPSI-IHeptCl 3-chloropyridine (41.9 mg; 0.043 mmol; 0.10 eq.) and Cs₂CO₃(280 mg; 0.860 mmol; 2.00 eq.) in 1,4-dioxane (7.0 mL) was stirred for 2 h at 100° C. under nitrogen atmosphere. The desired product could be detected by LCMS. The resulting mixture was cooled to room temperature and concentrated under reduced pressure. The residue was purified by flash chromatography on silica gel column using EtOAc in petroleum ether (35-60%) as eluent to provide 2-chloro-7-(3,3-difluoroazetidin-1-yl)-[1,2,4]triazolo[1,5-a]pyridine as a white solid (103 mg, 96%). LCMS (ESI) m/z 245.0, [M+H]⁺.

### Step 2: N-(5-(7-(3,3-difluoroazetidin-1-yl)-[1,2,4]triazolo[1,5-a]pyridin-2-yl)-8-(methylamino)-2,7-naphthyridin-3-yl)cyclopropanecarboxamide

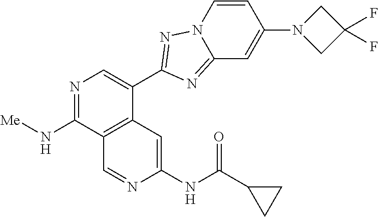 [SMILES: CNc1ncc(-c2nc3cc(N4CC(F)(F)C4)ccn3n2)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (100.0 mg; 0.272 mmol; 1.00 eq.), 2-chloro-7-(3,3-difluoroazetidin-1-yl)-[1,2,4]triazolo[1,5-a]pyridine (53.0 mg; 0.217 mmol; 0.80 eq.), XPhos Pd G₃ (22.3 mg; 0.026 mmol; 0.10 eq.), XPhos (12.9 mg; 0.027 mmol; 0.10 eq.) and K₃PO₃ (172.8 mg; 0.814 mmol; 3.00 eq.) in a mixture solvent of 1,4-dioxane/water (5:1, 2.4 mL) was stirred for 2 h at 90° C. under nitrogen atmosphere. The solvent was cooled to room temperature and concentrated, and the residue was purified by flash chromatography on silica gel column using MeOH in CH₂Cl₂ (0-20%) as eluent to give the crude product. The product was re-purified by C18 column using 20-80% of MeCN in water (10 mmol/L NH₄HCO₃) as eluent to provide N-(5-(7-(3,3-difluoroazetidin-1-yl)-[1,2,4]triazolo[1,5-a]pyridin-2-yl)-8-(methylamino)-2,7-naphthyridin-3-yl)cyclopropanecarboxamide as a white solid (14.1 mg, 12%). LCMS (ESI) m/z 451.2, [M+H]⁺. ¹H NMR (400 MHz, DMSO-d₆) δ 10.94 (s, 1H), 9.48 (s, 1H), 9.40 (s, 1H), 8.77 (s, 1H), 8.73 (d, J=7.2 Hz, 1H), 8.29-8.23 (m, 1H), 6.67 (d, J=2.0 Hz, 1H), 6.57 (dd, J=7.2, 2.0 Hz, 1H), 4.50 (t, J=12.4 Hz, 4H), 3.05 (d, J=4.4 Hz, 3H), 2.11-2.04 (m, 1H), 0.89-0.79 (m, 4H).

### Example 190: Synthesis of N-(5-(7-(3-(3,3-difluoroazetidin-1-yl)piperidin-1-yl)-[1,2,4]triazolo[1,5-a]pyridin-2-yl)-8-(methylamino)-2,7-naphthyridin-3-yl)cyclopropanecarboxamide

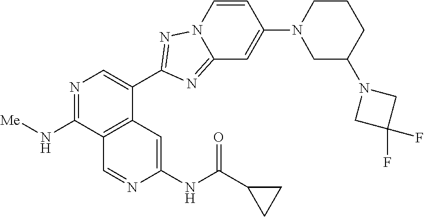 [SMILES: CNc1ncc(-c2nc3cc(N4CCCC(N5CC(F)(F)C5)C4)ccn3n2)c2cc(NC(=O)C3CC3)ncc12]

### Step 1: tert-butyl 3-(3,3-difluoroazetidin-1-yl)piperidine-1-carboxylate

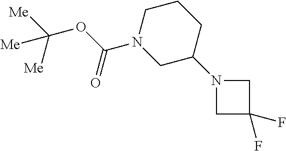 [SMILES: CC(C)(C)OC(=O)N1CCCC(N2CC(F)(F)C2)C1]

A mixture of tert-butyl 3-oxopiperidine-1-carboxylate (1.0 g; 5.02 mmol; 1.00 eq.), 3,3-difluoroazetidine hydrochloride (1.3 g; 10.0 mmol; 2.00 eq.) and DIPEA. (1.3 g; 10.0 mmol; 2 eq.) in CH₂Cl₂ (20.0 mL) was stirred for 2 h at room temperature under nitrogen atmosphere. To this mixture was added NaBH(OAc)₃ (3.2 g; 15.1 mmol; 3.00 eq.). The reaction was monitored by TLC. The resulting mixture was diluted with CH₂Cl₂ (200 mL). The organic layers were washed with brine (3×30 mL), dried over anhydrous Na₂SO₄. After filtration, the filtrate was concentrated under reduced pressure. The residue was purified by flash chromatography on silica gel column using 30-60% of EtOAc in petroleum ether as eluent to provide ten-butyl 3-(3,3-difluoroazetidin-1-yl)piperidine-1-carboxylate as a brown oil (1.1 g; 79%). LCMS (ESI) m/z 277.2, [M+H]⁺.

### Step 2: 3-(3,3-difluoroazetidin-1-yl)piperidine hydrochloride

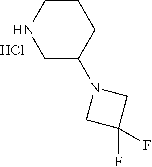 [SMILES: Cl ; FC1(F)CN(C2CCCNC2)C1]

A mixture of ten-butyl 3-(3,3-difluoroazetidin-1-yl)piperidine-1-carboxylate (200 mg; 0.724 mmol; 1.00 eq.) in HCl (gas)/dioxane (4M, 10 mL) was stirred for 1 h at room temperature. The desired product could be detected by LCMS. Upon completion, the resulting mixture was concentrated under reduced pressure to afford 3-(3,3-difluoroazetidin-1-yl)piperidine hydrochloride as a yellow solid (102 mg, crude), LCMS (ESI) m/z 177.1.

### Step 3: 2-chloro-7-(3-(3,3-difluoroazetidin-1-yl)piperidin-1-yl)-[1,2,4]triazolo[1,5-a]pyridine

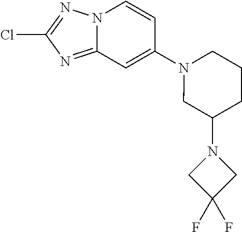 [SMILES: FC1(F)CN(C2CCCN(c3ccn4nc(Cl)nc4c3)C2)C1]

A mixture of 3-(3,3-difluoroazetidin-1-yl)piperidine hydrochloride (91.5 mg; 0.430 mmol; 1.00 eq.), 7-bromo-2-chloro-[1,2,4]triazolo[1,5-a]pyridine (100 mg; 0.430 mmol; 1.00 eq.), Pd-PEPPSI-IHeptCl, 3-chloropyridine (41.9 mg; 0.043 mmol; 0.10 eq.) and Cs₂CO₃ (280 mg; 0.859 mmol; 2.00 eq.) in 1,4-dioxane (10.0 ML) was stirred for 2 h at 100° C. under nitrogen atmosphere. The desired product could be detected by LCMS. The resulting mixture was concentrated under reduced pressure. The residue was purified by flash chromatography on silica gel column using 25-60% of EtOAc in petroleum ether as eluent to provide 2-chloro-7-(3-(3,3-difluoroazetidin-1-yl)piperidin-1-yl)-[1,2,4]triazolo[1,5-a]pyridine as a yellow solid (27.0 mg, 19%). LCMS (ESI) m/z 328.1, [M+H]⁺.

### Step 4: N-(5-(7-(3-(3,3-difluoroazetidin-1-yl)piperidin-1-yl)-[1,2,4]triazolo[1,5-a]pyridin-2-yl)-8-(methylamino)-2,7-naphthyridin-3-yl)cyclopropanecarboxamide

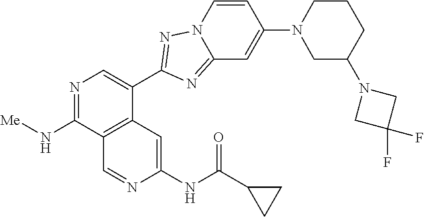 [SMILES: CNc1ncc(-c2nc3cc(N4CCCC(N5CC(F)(F)C5)C4)ccn3n2)c2cc(NC(=O)C3CC3)ncc12]

A mixture of N-(8-(methylamino)-5-(4,4,5,5-tetramethyl-1,3,2-dioxaborolan-2-yl)-2,7-(50.0 mg; 0.081 mmol; 0.99 eq.), 2-chloro-7-(3-(3,3-difluoroazetidin-1-yl)piperidin-1-yl)[1,2,4]triazolo[1,5-a]pyridine (27.0 mg; 0.082 mmol; 1.00 eq.), XPhos Pd G₃ (7.0 mg; 0.008 mmol; 0.10 eq.), XPhos (3.9 mg; 0.008 mmol; 0.10 eq.) and K₃PO₄ (52.0 mg; 0.245 mmol; 2.97 eq.) in a mixture solvent of 1,4-dioxane/water (5:1, 1.4 mL) was stirred for 1 h at 90° C. under nitrogen atmosphere. The desired product could be detected by LCMS. The resulting mixture was cooled to room temperature and concentrated under reduced pressure. The residue was purified by flash chromatography on silica gel column using Maki, in CH₂Cl₂ (0-20%) as eluent to give the crude product. The product was re-purified by C18 column using 40-60% of MeCN in water (10 mmol/L NH₄HCO₃) as eluent to provide N-(5-(7-(3-(3,3-difluoroazetidin-1-yl)piperidin-1-yl)-[1,2,4]triazolo[1,5-a]pyridin-2-yl)-8-(methylamino)-2,7-naphthyridin-3-yl)cyclopropanecarboxamide as a yellow solid (4.5 mg, 10%). LCMS (ESI) m/z 534.2, [M+H]⁺. ¹H NMR (400 MHz, DMSO-d₆) δ 10.92 (s, 1H), 9.45 (s, 1H), 9.40 (s, 1H), 8.73 (s, 1H), 8.58 (d, J=7.6 Hz, 1H), 8.34-8.21 (m, 1H), 7.01-6.93 (m, 2H), 3.79-3.69 (m, 6H), 3.05 (d, J=4.4 Hz, 3H), 3.02-2.92 (m, 1H), 2.81-2.72 (m, 1H), 2.47-2.39 (m, 1H), 2.11-2.02 (m, 1H), 1.88-1.74 (m, 2H), 1.59-1.46 (m, 1H), 1.32-1.18 (m, 1H), 0.89-0.79 (m, 4H).

### Example 191: Synthesis of N-(5-(6-fluoro-[1,2,4]triazolo[1,5-a]pyridin-2-yl)-8-(methylamino)-2,7-naphthyridin-3-yl)cyclopropanecarboxamide

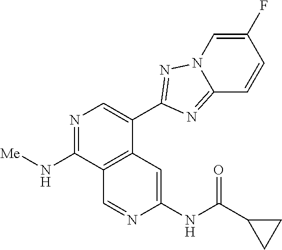 [SMILES: CNc1ncc(-c2nc3ccc(F)cn3n2)c2cc(NC(=O)C3CC3)ncc12]

### Step 1: 2-chloro-6-fluoro-[1,2,4]triazolo[1,5-a]pyridine

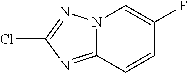 [SMILES: Fc1ccc2nc(Cl)nn2c1]

To a stirred solution of 6-fluoro-[1,2,4]triazolo[1,5-a]pyridin-2-amine (46.0 mg; 0.302 mmol; 1.0 eq.) and CuCl₂ (11.0 mg; 0.082 mmol; 0.27 eq.) in conc. HCl (0.5 mL) was added dropwise solution of NaNO₂ (25.0 mg; 0.362 mmol; 1.20 eq.) in water (0.2 mL) at 5° C. The resulting mixture was stirred for 30 min at 5° C. and then overnight at room temperature. The desired product could be detected by LCMS. The resulting mixture was diluted with water (10 mL). The resulting mixture was extracted with EtOAc (2×10 mL). The combined organic layers were washed with brine (20 mL) and dried over anhydrous Na₂SO₄. After filtration, the filtrate was concentrated under reduced pressure to afford 2-chloro-6-fluoro-[1,2,4]triazolo[1,5-a]pyridine as a white solid (50.0 mg, 96%). LCMS (ESI) m/z 172.0, [M+H]⁺.

### Step 2: N-(5-(6-fluoro-[1,2,4]triazolo[1,5-a]pyridin-2-yl)-8-(methylamino)-2,7-naphthyridin-3-yl)cyclopropanecarboxamide

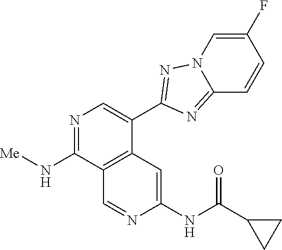 [SMILES: CNc1ncc(-c2nc3ccc(F)cn3n2)c2cc(NC(=O)C3CC3)ncc12]

Under nitrogen atmosphere, N-(8-(methylamino)-5-(4,4,5,5-tetramethyl-1,3,2-dioxaborolan-2-yl)-2,7-naphthyridin-3-yl)cyclopropanecarboxamide (100.0 mg; 0.272 mmol; 1.0 eq.), 2-chloro-6-fluoro-[1,2,4]triazolo[1,5-a]pyridine (46.5 mg; 0.272 mmol; 1.0 eq.), XPhos Pd G₃ (22.9 mg; 0.027 mmol; 0.1 eq.), XPhos (25.9 mg; 0.054 mmol; 0.2 eq.) and K₃PO₄(115.2 mg; 0.544 mmol, 2.0 eq.) were mixed in 1,4-dioxane/water (5:1, 2.4 mL). The reaction was stirred for 1 h at 90° C. The resulting mixture was cooled to room temperature and concentrated under reduced pressure. The residue was purified by flash chromatography on silica gel column using methanol in dichloromethane (3-7%) as eluent to give the crude product. The product was re-purified by flash chromatography on pre-packed C18 column using 20-49% of MeCN in water (10 mmol/L NH₄HCO₃) as eluent to provide N-(5-(6-fluoro-[1,2,4]triazolo[1,5-a]pyridin-2-yl)-8-(methylamino)-2,7-naphthyridin-3-yl)cyclopropanecarboxamide as a white solid (6.4 mg, 6.2%), LCMS (ESI) m/z 378.1, [M+H]⁺. ¹H NMR (400 MHz, DMSO-d₆) δ 10.94 (s, 1H), 9.43 (s, 1H), 9.41 (s, 1H), 9.33-9.20 (m, 1H), 8.77 (s, 1H), 8.33-8.28 (m, 1H), 7.96-7.91 (m, 1H), 7.84-7.77 (m, 1H), 3.06 (d, J=4.4 Hz, 3H), 2.11-2.03 (m, 1H), 0.88-0.79 (m, 4H)

### Example 192: Synthesis of N-(5-(5-fluoro-[1,2,4]triazolo[1,5-a]pyridin-2-yl)-8-(methylamino)-2,7-naphthyridin-3-yl)cyclopropanecarboxamide

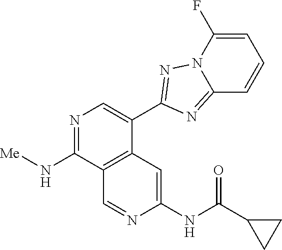 [SMILES: CNc1ncc(-c2nc3cccc(F)n3n2)c2cc(NC(=O)C3CC3)ncc12]

### Step 1: 2-chloro-5-fluoro-[1,2,4]triazolo[1,5-a]pyridine

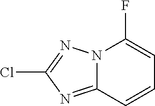 [SMILES: Fc1cccc2nc(Cl)nn12]

To a stirring mixture of 5-fluoro-[1,2,4]triazolo[1,5-a]pyridin-2-amine (46.0 mg; 0.302 mmol; 1.00 eq.) and CuCl₂ (11.0 mg; 0.173 mmol; 0.57 eq.) in cone. (0.5 mL) was added dropwise a solution of NaNO₂ (25.0 mg; 0.362 mmol; 1.20 eq.) in water (0.2 mL) at 5° C. The resulting mixture was stirred for 30 min at 5° C. under air atmosphere and then overnight at room temperature. Upon completion, the resulting mixture was diluted with water (20 mL). The resulting mixture was extracted with EtOAc (3×10 mL). The combined organic layers were washed with brine (10 mL), dried over anhydrous Na₂SO₄. After filtration, the filtrate was concentrated under reduced pressure to afford 2-chloro-5-fluoro-[1,2,4]triazolo[1,5-a]pyridine as a white solid (48 mg, 92%). LCMS (ESI) m/z 172.0, [M+H]⁺.

### Step 2: N-(5-(5-fluoro-[1,2,4]triazolo[1,5-a]pyridin-2-yl)-8-(methylamino)-2,7-naphthyridin-3-yl)cyclopropanecarboxamide

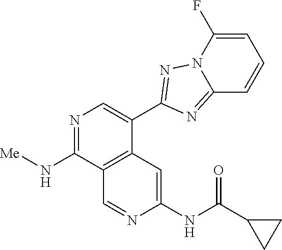 [SMILES: CNc1ncc(-c2nc3cccc(F)n3n2)c2cc(NC(=O)C3CC3)ncc12]

Under nitrogen atmosphere, N-(8-(methylamino)-5-(4,4,5,5-tetramethyl-1,3,2-dioxaborolan-2-yl)-2,7-naphthyridin-3-yl)cyclopropanecarboxamide (100.0 mg; 0.272 mmol; 1.0 eq.), 2-chloro-5-fluoro-[1,2,4]triazolo[1,5-a]pyridine (46.6 mg; 0.272 mmol; 1.0 eq.), XPhos Pd 3 (22.9 mg; 0.027 mmol; 0.1 eq.), XPhos (25.9 mg; 0.054 mmol; 0.2 eq.) and K₃PO₄ (115 mg; 0.544 mmol; 2.0 eq.) in a mixture solvent of 1,4-dioxane/water (5:1, 2.4 mL). The reaction was stirred for 1 h at 90° C. The resulting mixture was cooled to morn temperature and concentrated under reduced pressure. The residue was purified by flash chromatography on silica gel column using methanol in dichloromethane (3-7%) as eluent to give 21.5 mg crude product. The product was re-purified by flash chromatography on pre-packed C18 column using 20-49% of MeCN in water (10 mmol/L NH₄HCO₃) as eluent to provide N-(5-(5-fluoro-[1,2,4]triazolo[1,5-a]pyridin-2-yl)-8-(methylamino)-2,7-naphthyridin-3-yl)cyclopropanecarboxamide as a white solid (8.2 mg, 7.5%), LCMS (ESI) m/z 378.1, [M+H]⁺. ¹H NMR (400 MHz, DMSO-d₆) δ 10.93 (s, 1H), 9.55 (s, 1H), 9.42 (s, 1H), 8.86 (s, 1H), 8.40-8.30 (m, 1H), 7.85-7.68 (m, 2H), 7.19-7.08 (m, 1H), 3.07 (d, J=4.4 Hz, 3H), 2.15-1.99 (m, 1H), 0.95-0.74 (m, 4H.

### Example 193: Synthesis of N-(5-(8-fluoro-[1,2,4]-triazolo[1,5-a]pyridin-2-yl)-8-(methylamino)-2,7-naphthyridin-3-yl)cyclopropanecarboxamide

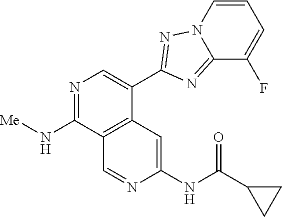 [SMILES: CNc1ncc(-c2nc3c(F)cccn3n2)c2cc(NC(=O)C3CC3)ncc12]

### Step 1: 2-chloro-8-fluoro-[1,2,4]triazolo[1,5-a]pyridine

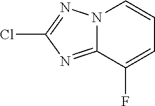 [SMILES: Fc1cccn2nc(Cl)nc12]

To a cooled, stirred suspension of 8-fluoro-[1,2,4]triazolo[1,5-a]pyridin-2-amine (200 mg; 1.315 mmol; 1.00 eq.) and CuCl₂ (46 mg; 0.342 mmol; 0.26 eq.) in a solution of hydrochloric acid (12M, 2.93 mL) at 5° C. was added dropwise a solution of NaNO₂ (109 mg; 1.580 mmol; 1.20 eq.) in water (0.6 mL). The mixture was stirred for 30 minutes at 5° C. then at room temperature for 18 hours. The yellow mixture was diluted with water (80 and the resulting precipitate was filtered, rinsed with water and dried to provide 2-chloro-8-fluoro-[1,2,4]triazolo[1,5-a]pyridine as a white solid (121 mg, 53%). LCMS m/z 172.0, [M+H]⁺.

### Step 2: N-(5-(8-fluoro-[1,2,4]triazolo[1,5-a]pyridin-2-yl)-8-(methylamino)-2,7-naphthyridin-3-yl)cyclopropanecarboxamide

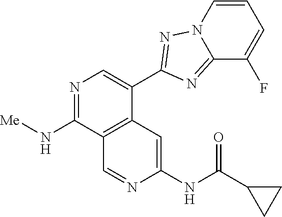 [SMILES: CNc1ncc(-c2nc3c(F)cccn3n2)c2cc(NC(=O)C3CC3)ncc12]

To a mixture of N-(8-(methylamino)-5-(4,4,5,5-tetramethyl-1,3,2-dioxaborolan-2-yl)-2,7-naphthyridin-3-yl)cyclopropanecarboxamide (150 mg; 0.407 mmol; 1.00 eq.) and 2-chloro-8-fluoro-[1,2,4]triazolo[1,5-a]pyridine (69.7 mg; 0.406 mmol; 1.00 eq.) in a mixture solvent of 1,4-dioxane/water (10:1, 3.3 mL) were added XPhos (38.8 mg; 0.081 mmol; 0.20 eq.), XPhos Pd G₃ (69 mg; 0.082 mmol; 0.20 eq.) and K₃PO₄ (172.8 mg; 0.814 mmol; 2.00 eq.). After stirring for 1 h at 90° C. under a nitrogen atmosphere. The resulting mixture was cooled to room temperature and concentrated under reduced pressure. The residue was purified by flash chromatography on silica gel column using MeOH in CH₂Cl₂ (2-10%) as eluent to provide 50 mg of crude product. The product was re-purified by flash chromatography on pre-packed C18 column using 20-50% of MeCN in water (10=ion NH₄HCO₃) as eluent to provide N-(5-(8-fluoro-[1,2,4]triazolo[1,5-a]pyridin-2-yl)-8-(methylamino)-2,7-naphthyridin-3-yl)cyclopropanecarboxamide as a white solid (18.8 mg, 12%). LCMS (ESI) m/z 378.1, [M+H]⁺. 1H NMR (400 MHz, DMSO-d₆) δ 10.96 (5, 1H), 9.50 (5, 1H), 9.42 (5, 1H), 8.87-8.82 (m, 2H), 8.38-8.32 (m, 1H), 7.67-7.60 (m, 1H), 7.21-7.14 (m, 1H), 3.06 (d, J=4.4 Hz, 3H), 2.12-2.03 (m, 1H), 0.89-0.78 (m, 4H).

### Example 194: Synthesis of N-(5-(7-fluoro-[1,2,4]triazolo[1,5-a]pyridin-2-yl)-8-(methylamino)-2,7-naphthyridin-3-yl)cyclopropanecarboxamide

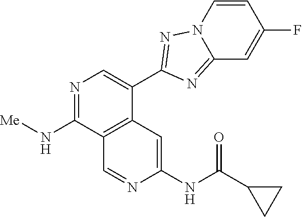 [SMILES: CNc1ncc(-c2nc3cc(F)ccn3n2)c2cc(NC(=O)C3CC3)ncc12]

### Step 1: 2-chloro-7-fluoro-[1,2,4]triazolo[1,5-a]pyridine

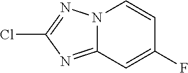 [SMILES: Fc1ccn2nc(Cl)nc2c1]

A mixture of 7-fluoro-[1,2,4]triazolo[1,5-a]pyridin-2-amine (200 mg; 1,315 mmol; 1.00 eq.) and CuCl₂ (45.9 mg; 0.342 mmol; 0.26 eq.) in 12 M HCl (2.9 mL) was stirred for 5 min at 5° C. Then NaNO₂ (108.8 mg; 1.578 mmol; 1.2 eq.) in water (0.6 mL) was added dropwise and stirred for 0.5 h at 5° C. then 12 h at room temperature under air atmosphere. The desired product could be detected by LCMS. The resulting mixture was diluted with water (50 mL). The resulting mixture was extracted with EtOAc (3×20 mL). The combined organic layers were washed with brine (1×5 mL) and dried over anhydrous Na₂SO₄. After filtration, the filtrate was concentrated under reduced pressure to afford 2-chloro-7-fluoro-[1,2,4]triazolo[1,5-a]pyridine as a off-white solid (170 mg, 75%). LCMS (ESI) m/z 172.0, [M+H]⁺.

### Step 2: N-(5-(7-fluoro-[1,2,4]triazolo[1,5-a]pyridin-2-yl)-8-(methylamino)-2,7-naphthyridin-3-yl)cyclopropanecarboxamide

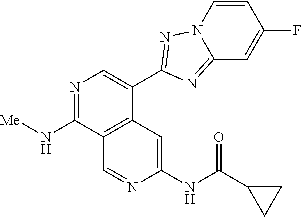 [SMILES: CNc1ncc(-c2nc3cc(F)ccn3n2)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (140 mg; 0.380 mmol; 1.00 eq.), XPhos Pd G₃ (64.3 mg; 0.076 mmol; 0.20 eq.), XPhos (44.5 mg; 0.076 mmol; 0.20 eq.) and K₃PO₄ (242.1 mg; 1.140 mmol; 3.00 eq.) in a mixture solvent of 1,4-dioxane/water (10:1, 5.5 mL) was stirred at 90° C. for 1 h under nitrogen atmosphere. The reaction was monitored by LCMS. The resulting mixture was cooled to room temperature and concentrated under reduced pressure. The residue was purified by flash chromatography on silica gel column using MeOH in CH₂Cl₂ (5-10%) as eluent to afford the crude product. The product was re-purified by flash chromatography on pre-packed C18 column using 20-50% of MeCN in water (10 mmol/L NH₄HCO₃) as eluent to provide N-(5-(7-fluoro-[1,2,4]triazolo[1,5-a]pyridin-2-yl)-8-(methylamino)-2,7-naphthyridin-3-yl)cyclopropanecarboxamide as a white solid (4.2 mg, 3%). LCMS (ESI) m/z 378.1, [M+H]⁺. NMR (400 MHz, DMSO-d₆) δ 10.94 (s, 1H), 9.48 (s, 1H), 9.41 (s, 1H), 9.05-9.00 (m, 1H), 8.81 (s, 1H), 8.35-8.29 (m, 1H), 7.80-7.74 (m, 1H), 7.24-7.18 (m, 1H), 3.06 (d, J=4.4 Hz, 3H), 2.12-2.13 (m, 1H), 0.89-0.78 (m, 4H).

### Example 195: Synthesis of N-(5-(5-(methoxy-d₃)benzo[d]oxazolo-naphthyridin-3-yl)cyclopropanecarboxamide

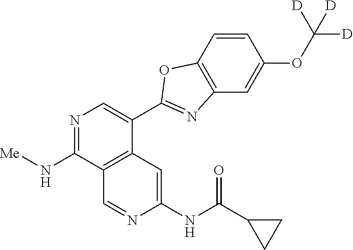 [SMILES: [2H]C([2H])([2H])Oc1ccc2oc(-c3cnc(NC)c4cnc(NC(=O)C5CC5)cc34)nc2c1]

### Step 1: 5-(methoxy-d₃)benzo[d]oxazole

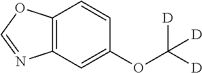 [SMILES: [2H]C([2H])([2H])Oc1ccc2ocnc2c1]

To a solution of benzo[d]oxazol-5-ol (250.0 mg; 1.85 mmol; 1.00 eq.) and Cs₂CO₃(1808 mg; 5.55 mmol; 3.00 eq.) in DMF (5 mL) was added iodomethane-d₃(804.6 mg; 5.55 mmol; 3.00 eq.). The resulting solution was stirred at 60° C. for 14 hours under nitrogen atmosphere. The solution was diluted with water (10 mL) and extracted with EtOAc (3×50 mL). The combined organic layers were washed with brine (3×25 mL), dried over anhydrous Na₂SO₄, filtered, and concentrated. The residue was purified via a silica gel column using 10-30% of EtOAc in petroleum ether as eluent to provide 5-(methoxy-d₃)benzo[d]oxazole as a pink solid (240.0 mg, 81.0%). LCMS (ESI) m/z 153.1, [M+H]⁺.

### Step 2: 2-chloro-5-(methoxy-d₃)benzo[d]oxazole

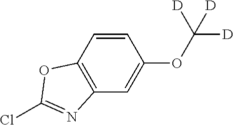 [SMILES: [2H]C([2H])([2H])Oc1ccc2oc(Cl)nc2c1]

To a solution of 5-(methoxy-d₃)benzo[d]oxazole (150 mg; 0.99 mmol; 1.00 eq.) in THF (20 mL) was added LiHMDS (1 M in THF, 1.2 mL) dropwise at −78° C. The resulting solution was stirred at −78° C. for 1 hour. To the above mixture was added hexachloroethane (466.7 mg; 1.972 mmol; 2.00 eq.) in portions at −78° C. The resulting mixture was stirred at room temperature for 0.5 hour. The reaction was quenched by a saturated NH₄Cl aqueous solution (10 mL), extracted with EtOAc (3×50 mL). The combined organic layers were washed with brine (3<10 mL), dried over anhydrous Na₂SO₄, filtered, and concentrated under reduced pressure. The residue was purified via a silica gel column using 10-30% of EtOAc in petroleum ether as eluent to provide 2-chloro-5-(methoxy-d₃)benzo[d]oxazole as a yellow solid (113 mg, 61.4%). LCMS (ESI) m/z 187.0, [M+H]⁺.

### Step 3: N-(5-(5-(methoxy-d₃)benzo[d]oxazol-2-yl)-8-(methylamino)-2,7-naphthyridin-3-yl)cyclopropanecarboxamide

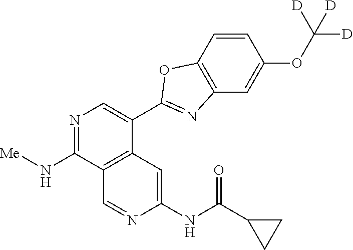 [SMILES: [2H]C([2H])([2H])Oc1ccc2oc(-c3cnc(NC)c4cnc(NC(=O)C5CC5)cc34)nc2c1]

A mixture of 2-chloro-5-(methoxy-d₃)benzo[d]oxazole (96.0 mg; 0.51 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (189.5 mg; 0.52 mmol; 1.00 eq.) in a mixture solvent of 1,4-dioxane/water (5:1, 5.4 mL). To the above solution were added XPhos Pd G₃ (43.6 mg; 0.052 mmol; 0.10 eq.), XPhos (24.5 mg; 0.051 mmol; 0.10 eq.) and K₃PO₄ (327.6 mg; 1.543 mmol; 3.00 eq.). The resulting mixture was stirred at 90° C. for 1 hour under nitrogen atmosphere. The solvent was concentrated under reduced pressure. The residue was purified via a silica gel column using 2-10% of MeOH in CH₂Cl₂ to afford a crude product. The crude product was further purified by flash chromatography on pre-packed C18 column using 20-60% of MeCN in water (10 mmol/L NH₄HCO₃) to provide N-(5-(5-(methoxy-d₃)benzo[d]oxazol-2-yl)-8-(methylamino)-2,7-naphthyridin-3-yl)cyclopropanecarboxamide as a yellow solid (29.8 mg, 14.7%). LCMS (ESI) m/z 393.2, [M+H]⁺. ¹H NMR (400 MHz, DMSO-d₆) 11.04 (s, 1H), 9.65 (s, 1H), 9.44 (s, 1H), 8.90 (s, 1H), 8.75-8.49 (m, 1H), 7.62 (d, J=8.8 HZ, 1H), 7.28 (d, J=2.4 Hz, 1.14), 7.01-6.81 (dd, J=8.8, 2.4 Hz, 1H), 3.09 (d, J=4.4 Hz, 3H), 2.19-2.02 (m, 1H), 1.00-0.76 (m, 4H).

### Example 196: Synthesis of tert-butyl 3-((2-(6-(cyclopropanecarboxamido)-1-(methylamino)-2,7-naphthyridin-4-yl)benzo[d]oxazol-5-yl)oxy)azetidine-1-carboxylate

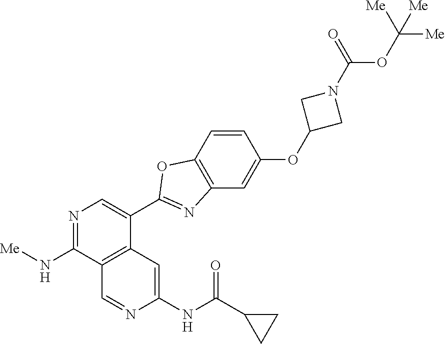 [SMILES: CNc1ncc(-c2nc3cc(OC4CN(C(=O)OC(C)(C)C)C4)ccc3o2)c2cc(NC(=O)C3CC3)ncc12]

### Step 1: text-butyl 3-(benzo[d]oxazol-5-yloxy)azetidine-1-carboxylate

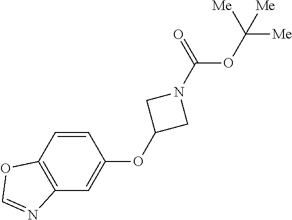 [SMILES: CC(C)(C)OC(=O)N1CC(Oc2ccc3ocnc3c2)C1]

To a solution of benzo[d]oxazol-5-ol (80.0 mg; 0.592 mmol; 1.00 eq.) and tert-butyl 3-bromoazetidine-1-carboxylate (419.3 mg; 1.776 mmol; 3.00 eq.) in DMF (5 mL) was added Cs₂CO₃(578.6 mg, 1.776 mmol 3.00 eq.) under nitrogen atmosphere. After stirring at 120° C. for 1 hour, the desired product was detected via LCMS. The resulting mixture was diluted with EtOAc (50 mL), washed with brine (3×10 ML). The organic layer was dried over anhydrous Na₂SO₄, filtered, and concentrated under reduced pressure. The crude product was purified by flash chromatography on silica gel column using 0-30% of EtOAc in petroleum ether as eluent to provide tort-butyl 3-(benzo[d]oxazol-5-yloxy)azetidine-1-carboxylate as a white solid (120.0 mg, 69.8%). LCMS (ESI) m/z 290.1, [M+H]⁺.

### Step 2: tert-butyl 3-((2-chlorobenzo[d]oxazol-5-yl)oxy)azetidine-1-carboxylate

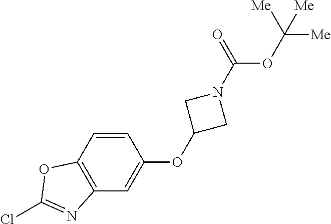 [SMILES: CC(C)(C)OC(=O)N1CC(Oc2ccc3oc(Cl)nc3c2)C1]

Teri-butyl 3-((2-chlorobenzo[d]oxazol-5-yl)oxy azetidine-1-carboxylate was synthesized using a similar procedure that was previously described in Example 195 by using tell-butyl 3-(benzo[d]oxazol-5-yloxy)azetidine-1-carboxylate and hexachloroethane as the starting material, LCMS (ESI) m/z 325.1.

### Step 3: tert-butyl 3-((2-(6-(cyclopropanecarboxamido)-1-(methylamino)-2,7-naphthyridin-4-yl)benzo[d]oxazol-5-yl)oxy)azetidine-1-carboxylate

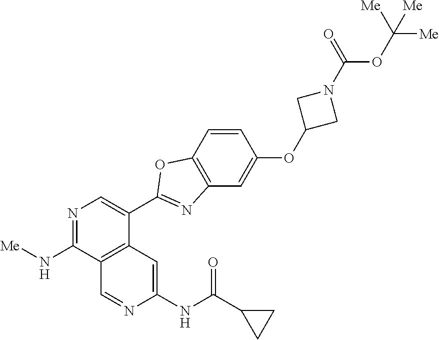 [SMILES: CNc1ncc(-c2nc3cc(OC4CN(C(=O)OC(C)(C)C)C4)ccc3o2)c2cc(NC(=O)C3CC3)ncc12]

tert-butyl 3-((2-(6-(cyclopropanecarboxamido)-1-(methylamino)-2,7-naphthyridin-4-yl)benzo[d]oxazol-5-yl)oxy)azetidine-1-carboxylate was synthesized using a similar procedure that was previously described in Example 195 by using N-(8-(methylamino)-5-(4,4,5,5-tetramethyl-1,3,2-dioxaborolan-2-yl)-2,7-naphthyridin-3-yl)cyclopropanecarboxamide and tert-butyl 3((2-chlorobenzo[d]oxazol-5-yl)oxy)azetidine-1-carboxylate as the starting material. LCMS (ESI) m/z 531.2, [M+H]⁺. ¹H NMR (400 MHz, DMSO-d₆) δ 11.05 (s, 1H), 9.58 (s, 1H), 9.44 (s, 1H), 8.89 (s, 1H), 8.70-8.62 (m, 1H), 7.65 (d, J=8.8 Hz, 1H), 7.15 (d, J=2.4 Hz, 1H), 6.97-6.89 (m, 1H), 5.17-5.07 (m, 1H), 4.41-4.28 (m, 2H), 3.90-3.80 (m, 2H), 3.09 (d, J=4.4 Hz 3H), 2.15-2.05 (m, 1H), 1.41 (s, 9H), 0.97-0.82 (m, 4H).

### Example 197: Synthesis of N-(5-(5-(azetidin-3-yloxy)benzo[d]oxazol-2-yl)-8-(methylamino)-2,7-naphthyridin-3-yl)cyclopropanecarboxamide

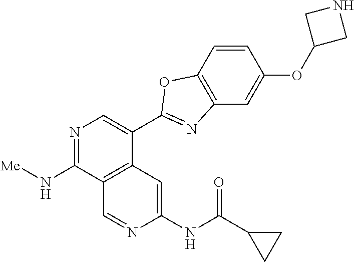 [SMILES: CNc1ncc(-c2nc3cc(OC4CNC4)ccc3o2)c2cc(NC(=O)C3CC3)ncc12]

To a stirring mixture of tert-butyl 3-((2-(6-(cyclopropanecarboxamido)-1-(methylamino)-2,7-naphthyridin-4-yl)benzo[d]oxazol-5-yl)oxy)azetidine-1-carboxylate (Example 196) (50.0 mg; 0.094 mmol; 1.00 eq.) in MeOH (1 mL) was added HCl (gas) (4 Min 1,4-dioxane, 2 mL) at 0° C. under nitrogen atmosphere. The resulting mixture was stirred at room temperature for 2 hours. The desired product was detected via LCMS. The resulting mixture was concentrated under reduced pressure. The residue was purified by flash chromatography on silica gel column using MeOH in CH₂Cl₂ (0-10%) as eluent to provide N-(5-(5-(azetidin-3-yloxy)benzo[d]oxazol-2-yl)-8-(methylamino)-2,7-naphthyridin-3-yl)cyclopropanecarboxamide as a yellow solid (2.1 mg, 5.1%). LCMS (ESI) m/z 431.2, [M+H]⁺. ¹H NMR (400 MHz, DMSO-d₆) 11.04 (s, 1H), 9.64 (s, 1H), 9.44 (s, 1H), 8.85 (s, 1H), 8.70-8.57 (m, 1.14), 7.63 (d, J=8.4 Hz, 1H), 7.08 (d, J=2.4 Hz, 1H), 6.97-6.81 (m, 1H), 5.14-5.03 (m, 1H), 3.92-3.75 (m, 2H), 3.62-3.51 (m, 2H), 3.09 (d, J=4.4 Hz, 3H), 2.16-2.04 (m, 1H), 0.98-0.79 (m, 4H).

### Example 198: Synthesis of N-(8-(methylamino)-5-(5-((1-methylazetidin-3-yl)oxy)benzo[d]oxazol-2-yl)-2,7-naphthyridin-3-yl)cyclopropanecarboxamide

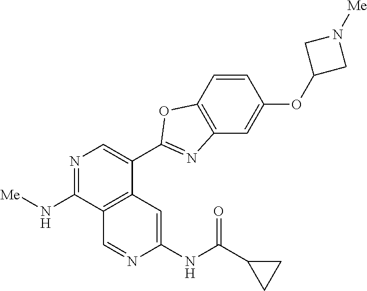 [SMILES: CNc1ncc(-c2nc3cc(OC4CN(C)C4)ccc3o2)c2cc(NC(=O)C3CC3)ncc12]

A solution of N-(5-(5-(azetidin-3-yloxy)benzo[d]oxazol-2-yl)-8-(methylamino)-2,7-naphthyridin-3-yl)cyclopropanecarboxamide (Example 197) (10.0 mg; 0.023 mmol; 1.00 eq.) and HCHO (37% w/w in water) (13.5 mg; 0.167 mmol; 7.17 eq.) in CH₂Cl₂ (2 mL) was stirred at room temperature for 2 hours under nitrogen atmosphere. To the above solution was added-NaBH(OAc)₃ (13.9 mg; 0.066 mmol; 2.84 eq.). The resulting mixture was stirred at room temperature for 2 hours. The desired product was detected via LCMS. The resulting mixture was concentrated under reduced pressure. The residue was purified by flash chromatography on silica gel column using MeOH in CH₂Cl₂ (0-10%) as eluent to provide N-(8-(methylamino)-5-(5-((1-methylazetidin-3-yl)oxy)benzo[d]oxazol-2-yl)-2,7-naphthyridin-3-yl)cyclopropanecarboxamide as a yellow solid (9.7 mg, 94.8%), LCMS (ESI) m/z 445.2, [M+H]⁺. ¹H NMR (400 MHz, DMSO-d₆) δ 11.04 (s, 1H), 9.58 (s, 1H), 9.44 (s, 1H), 8.88 (s, 8.69-8.61 (m, 1H), 7.62 (d, J=8.7 Hz, 1H), 7.11 (d, 2.5 Hz, 1H), 6.93-6.86 (m, 1H), 4.92-4.82 (m, 1H), 3.82-3.74 (m, 2H), 3.09 (d, J=4.4 Hz, R) 3H), 3.06-2.98 (m, 2H), 2.31 (s, 3H), 2.15-2.05 (m, 1H), 0.97-0.82 (m, 4H).

### Example 199: Synthesis of N-(5-(5-((1r,3r)-3-methoxycyclobutoxy)benzo[d]oxazol-2-yl)-8-(methylamino)-2,7-naphthyridin-3-yl)cyclopropanecarboxamide

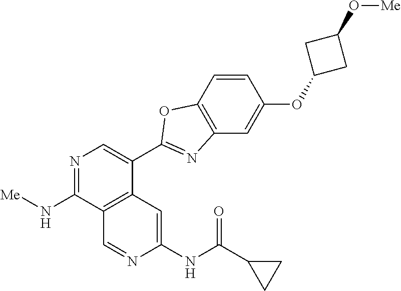 [SMILES: CNc1ncc(-c2nc3cc(O[C@H]4C[C@H](OC)C4)ccc3o2)c2cc(NC(=O)C3CC3)ncc12]

### Step 1: 5-((1r,3r)-3-methoxycyclobutoxy)benzo[d]oxazole

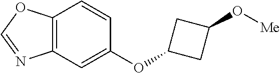 [SMILES: CO[C@H]1C[C@H](Oc2ccc3ocnc3c2)C1]

To a solution of benzo[d]oxazol-5-ol (110.0 mg; 0.814 mmol; 1.00 eq.) and (1s,3s)-3-methoxycyclobutan-1-ol (65.6 mg; 0.643 mmol; 0.79 eq.) in THF (15 mL) were added PPh₃ (234.8 mg; 0.895 mmol; 1.10 eq.) and DIAD (164.5 mg; 0.814 mmol; 1.00 eq.) under nitrogen atmosphere. After stirring at room temperature for 14 hours, the desired product was detected via LCMS. The resulting mixture was concentrated under reduced pressure. The residue was purified by flash chromatography on silica gel column using 0-50% of EtOAc in petroleum ether as eluent to provide 5-((1R,3R)-3-methoxycyclobutoxy)benzo[d]oxazole as a white solid (58.0 mg, 32.5%). LCMS (ESI) m/z 220.1, [M+H]⁺.

### Step 2: 2-chloro-5-((1r,3r)-3-methoxycyclobutoxy)benzo[d]oxazole

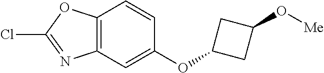 [SMILES: CO[C@H]1C[C@H](Oc2ccc3oc(Cl)nc3c2)C1]

2-chloro-5-((1r,3r)-3-methoxycyclobutoxy)benzo[d]oxazole was synthesized using a similar procedure that was previously described in Example 195 by using 5-((1r,3r)-3-methoxycyclobutoxy)benzo[d]oxazole and hexachloroethane as the starting material. LCMS (ESI) m/z 254.1, [M+H]⁺.

### Step 3: N-(5-(5-((1r,3r)-3-methoxycyclobutoxy)benzo[d]oxazol-2-yl)-8-(methylamino)-2,7-naphthyridin-3-yl)cyclopropanecarboxamide

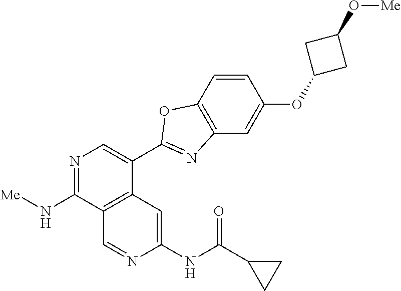 [SMILES: CNc1ncc(-c2nc3cc(O[C@H]4C[C@H](OC)C4)ccc3o2)c2cc(NC(=O)C3CC3)ncc12]

N-(5-(5-((1R,3R)-3-methoxycyclobutoxy)benzo[d]oxazol-2-yl)-8-(methylamino)-2,7-naphthyridin-3-yl)cyclopropanecarboxamide was synthesized using a similar procedure that was previously described in Example 195 by using N-(8-(methylamino)-5-(4,4,5,5-tetramethyl-1,3,2-dioxaborolan-2-yl)-2,7-naphthyridin-3-yl)cyclopropanecarboxamide and 2-chloro-5-((1r,3r)-3-methoxycyclobutoxy)benzo[d]oxazole as the starting material. LCMS (ESI) m/z 460.2, [M+H]⁺. ¹H NMR (400 MHz, DMSO-d₆) δ 11.05 (s, 1H), 9.59 (s, 1H), 9.44 (s, 1H), 8.88 (s, 1H), 8.69-8.60 (m, 1H), 7.62 (d, J=8.4 Hz, 1H), 7.10 (d, J=2.0 Hz, 1H), 6.93-6.86 (m, 1H), 4.98-4.90 (m, 1H), 4.16-4.06 (m, 1H), 3.19 (s, 3H), 3.09 (d, J=4.0 Hz, 3H), 2.52-2.4 ((m, 2H), 2.39-2.29 (m, 2H), 2.14-2.06 (m, 1H), 0.96-0.89 (m, 2H), 0.89-0.82 (m, 2H).

### Example 200: Synthesis of N-(5-(5-((1s,3s)-3-methoxycyclobutoxy)benzo[d]oxazol-2-yl)-8-(methylamino)-2,7-naphthyridin-3-yl)cyclopropanecarboxamide

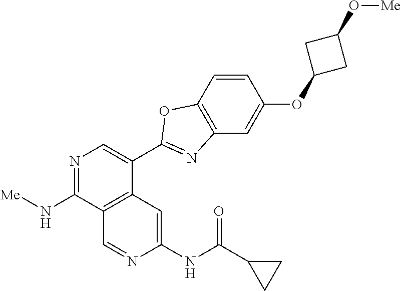 [SMILES: CNc1ncc(-c2nc3cc(O[C@H]4C[C@@H](OC)C4)ccc3o2)c2cc(NC(=O)C3CC3)ncc12]

### Step 1: 5-((1S,3S)-3-methoxycyclobutoxy)benzo[d]oxazole

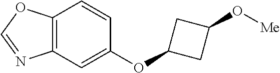 [SMILES: CO[C@H]1C[C@@H](Oc2ccc3ocnc3c2)C1]

To a solution of benzo[d]oxazol-5-ol (100.0 mg; 0.740 mmol; 1.00 eq.) and (1r,3r)-3-methoxycyclobutan-1-ol (59.9 mg; 0.587 mmol; 0.79 eq.) in THF (10 mL) were added PPh₃ (212.9 mg; 0.812 mmol; 1.10 eq.) and DIM) (150.0 mg; 0.742 mmol; 1.00 eq.) under nitrogen atmosphere. After stirring at 50° C. for 1 hour, the desired product was detected via LCMS. The resulting mixture was concentrated under reduced pressure. The residue was purified by flash chromatography on silica gel column using 0-50% of EtOAc in petroleum ether as eluent to provide 5-((1s,3s)-3-methoxycyclobutoxy)benzo[d]oxazole as a colorless oil (70.0 mg, 43.1%). LCMS (ESI) m/z 220.1, [M+H]⁺.

### Step 2: 2-chloro-5-((1s,3s)-3-methoxycyclobutoxy)benzo[d]oxazole

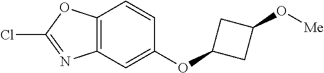 [SMILES: CO[C@H]1C[C@@H](Oc2ccc3oc(Cl)nc3c2)C1]

2-chloro-5-((1s,3s)-3-methoxycyclobutoxy)benzo[d]oxazole was synthesized using a similar procedure that was previously described in Example 195 by using 5-((1s,3s)-3-methoxycyclobutoxy)benzo[d]oxazole and hexachloroethane as the starting material. LCMS (ESI) m/z 254.1, [M+H]⁺.

### Step 3: N-(5-(5-((1S,3S)-3-methoxycyclobutoxy)benzo[d]oxazol-2-yl)-8-(methylamino)-2,7-naphthyridin-3-yl)cyclopropanecarboxamide

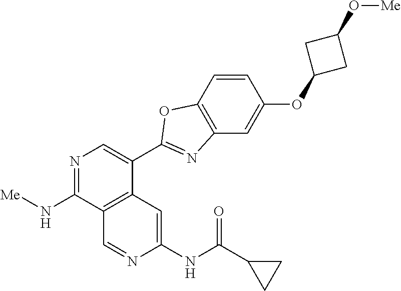 [SMILES: CNc1ncc(-c2nc3cc(O[C@H]4C[C@@H](OC)C4)ccc3o2)c2cc(NC(=O)C3CC3)ncc12]

N-(5-(5-((1S,3S)-3-methoxycyclobutoxy)benzo[d]oxazol-2-yl)-8-methylamino)-2,7-naphthyridin-3-yl)cyclopropanecarboxamide was synthesized using a similar procedure that was previously described in Example 195 by using N-(8-(methylamino)-5-(4,4,5,5-tetramethyl-3,2-dioxaborolan-2-yl)-2,7-naphthyridin-3-yl)cyclopropanecarboxamide and 2-chloro-5-((1s,3s)-3-methoxycyclobutoxy)benzo[d]oxazole as the starting material. LCMS (ESI) m/z 460.2, [M+H]⁺. ¹H NMR (400 MHz, DMSO-d₆) δ 11.04 (s, 1.14), 9.59 (s, 1H), 9.44 (s, 1H), 8.88 (s, 1H), 8.69-8.62 (m, 1H), 7.61 (d, J=8.8 Hz, 1H), 7.16 (d, J=2.4 Hz, 1H), 6.94-6.87 (m, 1H), 4.55-4.44 (m, 1H), 3.72-3.61 (m, 1H), 3.18 (s, 3H), 3.09 (d, J=4.4 Hz, 3H), 2.96-2.85 (m, 2H), 2.14-2.04 (m, 1H), 1.99-1.85 (m, 2H), 0.96-0.82 (m, 4H).

### Example 201: Synthesis of N-(5-(4-(methoxymethyl)benzo[d]oxazol-2-yl)-8-(methylamino)-2,7-naphthyridin-3-yl)cyclopropanecarboxamide

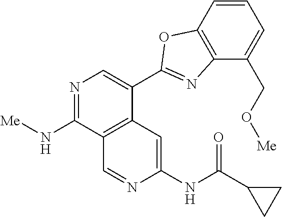 [SMILES: CNc1ncc(-c2nc3c(COC)cccc3o2)c2cc(NC(=O)C3CC3)ncc12]

### Step 1: 4-methylbenzo[d]oxazole

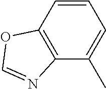

A mixture of 2-amino-3-methylphenol (5.01 g; 40.597 mmol; 1.00 eq.) and CH₃SO₃H (740.1 mg; 5.798 mmol; 0.20 eq.) in trimethyl orthoformate (35.7 mL) was stirred at room temperature for 2 hour under nitrogen atmosphere. Upon completion, the resulting mixture was concentrated under reduced pressure. The residue was purified by flash chromatography on silica gel column using 40-50% of EtOAc in petroleum ether as eluent to provide 4-methylbenzo[d]oxazole as a yellow solid (4.97 g, 91.9%). LCMS (ESI) m/z 134.1, [M+H]⁺.

### Step 2: 4-(bromomethyl)benzo[d]oxazole

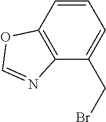 [SMILES: BrCc1cccc2ocnc12]

To a stirred solution of 4-methylbenzo[d]oxazole (4.32 g; 32.294 mmol; 1.00 eq.) in CCl₄ (50 mL) was added NBS (6.31 g; 35.391 mmol; 1.10 eq.) in batches and AIBN (240.3 mg; 1.461 mmol; 0.05 eq.). The resulting mixture was stirred at 40° C. for 4 hours under nitrogen atmosphere. The desired product was detected via LCMS. The resulting mixture was washed with water (2×10 mL). The organic layer was concentrated under reduced pressure. The residue was purified by flash chromatography on silica gel column using 10-30% of EtOAc in petroleum ether as eluent to provide 4-(bromomethyl)benzo[d]oxazole as an off-white solid (3.40 g, 50.1%). LCMS (ESI) m/z 212.0, [M+H]⁺.

### Step 3: 4-(methoxymethyl)benzo[d]oxazole

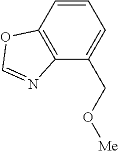 [SMILES: COCc1cccc2ocnc12]

A solution of 4-(bromomethyl)benzo[d]oxazole (1.51 g; 7.074 mmol; 1.00 eq.) and sodium methanolate (1.13 g; 20.917 mmol; 3.00 eq.) in MeOH (15 mL) was stirred at 60° C. overnight under nitrogen atmosphere. The desired product was detected via LCMS. The resulting mixture was diluted with water (50 mL) and acidified to pH=7 with 2 M HCl. The resulting mixture was extracted with EtOAc (3×20 mL). The combined organic layers were washed with brine (3×20 mL) and dried over anhydrous Na₂SO₄. After filtration, the filtrate was concentrated under reduced pressure. The residue was purified by flash chromatography on silica gel column using 10-30% of EtOAc in petroleum ether as eluent to provide 4-(methoxymethyl)benzo[d]oxazole as a yellow oil (900.2 mg, 77.9%). LCMS (ESI) m/z 164.1, [M+H]⁺.

### Step 4: 2-chloro-4-(methoxymethyl)benzo[d]oxazole

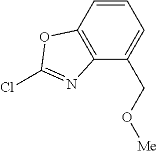 [SMILES: COCc1cccc2oc(Cl)nc12]

To a stirred solution of 4-(methoxymethyl)benzo[d]oxazole (100.2 mg; 0.612 mmol; 1.00 eq.) in THF (5 mL) was added LiHMDS (1 M in THF, 1 mL) dropwise at −78° C. After stirring at −78° C. for 1 hour, to the above mixture was added a solution of hexachloroethane (296.1 mg; 1.252 mmol; 2.00 eq.) in THF (0.5 mL). The resulting solution was stirred at −78° C. for another 1 hour. The desired product was detected via LCMS. The reaction was quenched with saturated NH₄Cl solution (10 mL) at 0° C. and extracted with EtOAc (3×20 mL). The combined organic layers were washed with brine (3×10 mL) and dried over anhydrous Na₂SO₄. After filtration, the filtrate was concentrated under reduced pressure. The residue was purified by flash chromatography on silica gel column using 10-30% of EtOAc in petroleum ether as eluent to provide 2-chloro-4-(methoxymethyl)benzo[s]oxazole as a colorless oil (60.1 mg, 49.5%). LCMS (ESI) m/z 198.0, [M+H]⁺.

### Step 5: N-(5-(4-(methoxymethyl)benzo[d]oxazol-2-yl)-8-(methylamino-2,7-naphthyridin-3-yl)cyclopropanecarboxamide

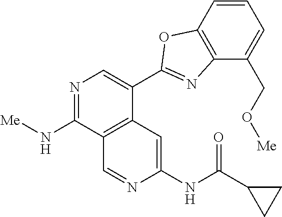 [SMILES: CNc1ncc(-c2nc3c(COC)cccc3o2)c2cc(NC(=O)C3CC3)ncc12]

To a stirred solution of 2-chloro-4-(methoxymethyl)benzo[d]oxazole (40.2 mg; 0.201 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (75.1 mg; 0.204 mmol; 1.00 eq.) in a mixture solvent of 1,4-dioxane/water (5:1, 1.8 mL) were added XPhos Pd G₃ (17.2 mg; 0.022 mmol; 0.01 eq.), XPhos (19.4 mg; 0.044 mmol; 0.02 eq.) and K₃PO₄ (129.0 mg; 0.602 mmol; 3.00 eq.). The resulting mixture was stirred at 90° C. for 1 hour under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide the crude product. The crude product was purified by flash chromatography on pre-packed C18 column using 50-60% of MeCN in water (10 mmol/L NH₄HCO₃) as eluent to provide N-(5-(4-(methoxymethyl)benzo[d]oxazol-2-yl)-8-(methylamino)-2,7-naphthyridin-3-yl)cyclopropanecarboxamide as a yellow solid (15.3 mg, 18.7%). LCMS (ESI) m/z 404.2, [M+H]⁺. ¹H NMR (400 MHz, DMSO-d₆) δ 11.04 (s, 1H), 10.01 (s, 1H), 9.44 (s, 1H), 8.93 (s, 1H), 8.68-8.63 (m, 1H), 7.70-7.63 (m, 1H), 7.42-7.33 (m, 2H), 4.87 (s, 2H), 3.43 (s, 3H), 3.10 (d, J=4.4 Hz, 3H), 2.16-2.06 (m, 1H), 0.95-0.82 (m, 4H).

### Example 202: Synthesis of N-(5-(5,6-dimethoxybenzo[d]oxazol-2-yl)-8-(ethylamino)-2,7-naphthyridin-3-yl)cyclopropanecarboxamide

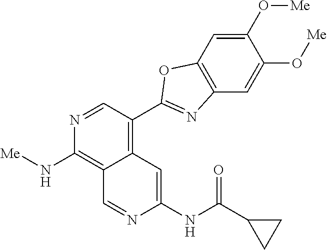 [SMILES: CNc1ncc(-c2nc3cc(OC)c(OC)cc3o2)c2cc(NC(=O)C3CC3)ncc12]

### Step 1: 5,6-dimethoxybenzo[d]oxazole-2(3H)-thione

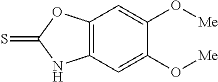 [SMILES: COc1cc2[nH]c(=S)oc2cc1OC]

A solution of 2-amino-4,5-dimethoxyphenol (150.0 mg; 0.729 mmol; 1.00 eq.) and potassium O-ethyl carbonodithioate (128.6 mg; 0.802 mmol; 1.10 eq.) in MeOH (3 mL) was stirred at 80° C. for 3 hours under nitrogen atmosphere. The resulting mixture was concentrated under reduced pressure. The residue was dissolved in water (10 mL), acidified to pH=4 with HCl (6 M). The precipitated solids were collected by filtration and washed with water (3×5 mL) to afford 5,6-dimethoxybenzo[d]oxazole-2(3H)-thione as a yellow solid (150.0 mg, 97.3%). LCMS (ESI) 212.0, [M+H]⁺.

### Step 2: 2-chloro-5,6-dimethoxybenzo[d]oxazole

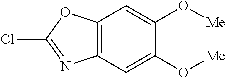 [SMILES: COc1cc2nc(Cl)oc2cc1OC]

To a stirred solution of 5,6-dimethoxybenzo[d]oxazole-2(3H)-thione (150.0 mg; 0.710 mmol; 1.00 eq.) in SOCl₂ (5 mL) was added DMF (0.1 mL; 0.411 mmol; 0.58 eq.) dropwise at 0° C. under nitrogen atmosphere. The resulting mixture was stirred at 80° C. for 0.5 hour. The resulting mixture was concentrated under reduced pressure to afford 2-chloro-5,6-dimethoxybenzo[d]oxazole as yellow solid (120 mg, crude). LCMS (ESI) m/z 214.0, [M+H]⁺.

Each intermediate in Table 9 below was prepared using a similar experimental procedure to prepare 2-chloro-5,6-dimethoxybenzo[d]oxazole, where 2-amino-4,5-dimethoxyphenol was replaced with the reagents as shown in Table 9 below.

TABLE 9

| Intermediate | Reagent | MS |
|---|---|---|
| 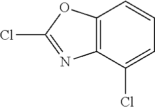 [SMILES: Clc1nc2c(Cl)cccc2o1] | 2-amino-3-chlorophenol | LCMS (ESI) m/z 188.0, [M + H]⁺. |
| 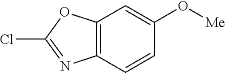 [SMILES: COc1ccc2nc(Cl)oc2c1] | 2-amino-5-methoxyphenol | LCMS (ESI) m/z 184.0, [M + H]⁺. |
| 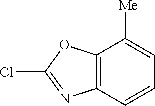 [SMILES: Cc1cccc2nc(Cl)oc12] | 2-amino-6-methylphenol | LCMS (ESI) m/z 168.0, [M + H]⁺. |
| 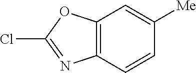 [SMILES: Cc1ccc2nc(Cl)oc2c1] | 2-amino-5-methylphenol | LCMS (ESI) m/z 168.0, [M + H]⁺. |
| 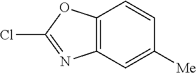 [SMILES: Cc1ccc2oc(Cl)nc2c1] | 2-amino-4-methylphenol | LCMS (ESI) m/z 168.0, [M + H]⁺. |

### Step 3: N-(5-(5,6-dimethoxybenzo[d]oxazol-2-yl)-8-(methylamino)-2,7-naphthyridin-3-yl)cyclopropanecarboxamide

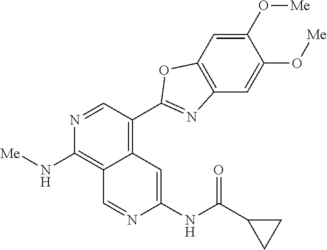 [SMILES: CNc1ncc(-c2nc3cc(OC)c(OC)cc3o2)c2cc(NC(=O)C3CC3)ncc12]

N-(5-(5,6-dimethoxybenzo[d]oxazol-2-yl)-8-(methylamino)-2,7-naphthyridin-3-yl)cyclopropanecarboxamide was synthesized using a similar procedure that was previously described in Example 195 by using N-(8-(methylamino)-5-(4,4,5,5-tetramethyl-1,3,2-dioxaborolan-2-yl)-2,7-naphthyridin-3-yl)cyclopropanecarboxamide and 2-chloro-5,6-dimethoxybenzo[d]oxazole as the starting material. LCMS (ESI) m/z 420.2, [M+H]⁺. ¹H NMR (400 MHz, DMSO-d₆) δ 11.02 (s, 1H), 9.64 (s, 1H), 9.43 (s, 1H), 8.83 (s, 1H), 8.68-8.40 (m, 1H), 7.44 (s, 1H), 7.31 (s, 1H), 3.86 (s, 6H), 3.08 (d, J=4.4 Hz, 3H), 2.19-2.07 (m, 1H), 1.00-0.77 (m, 4H).

### Examples 203-207

Each compound in Table 10 below was prepared using a similar experimental procedure to prepare Example 195, using N-(8-(methylamino)-5-(4,4,5,5-tetramethyl-1,3,2-dioxaborolan-2-yl)-2,7-naphthyridin-3-yl)cyclopropanecarboxamide as the common intermediate and appropriate 2-chlorobenzo[d]oxazole substrates.

TABLE 10

| Example | Structure and Name | MS and ¹H NMR |
|---|---|---|
| Example 203 | 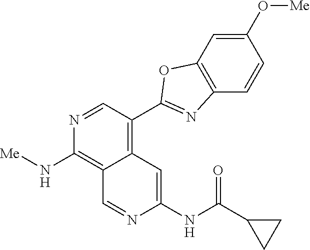 [SMILES: CNc1ncc(-c2nc3ccc(OC)cc3o2)c2cc(NC(=O)C3CC3)ncc12] | LCMS (ESI) m/z 390.1, [M + H]⁺. ¹H NMR (400 MHz, DMSO-d₆) δ 11.02 (s, 1H), 9.65 (s, 1H), 9.43 (s, 1H), 8.85 (s, 1H), 8.70-8.37 (m, 1H), 7.64 (d, J = 8.4 Hz, 1H), 7.37 (d, J = 2.0 Hz, 1H), 7.11-6.76 (m, 1H), 3.86 (s, 3H), 3.08 (d, J = 4.4 Hz, 3H), 2.18-2.01 (m, 1H), 1.00- 0.75 (m, 4H). |
| | N-(5-(6-methoxybenzo[d]oxazol-2-yl)- | |
| | 8-(methylamino)-2,7-naphthyridin-3- | |
| | yl)cyclopropanecarboxamide | |
| Example 204 | 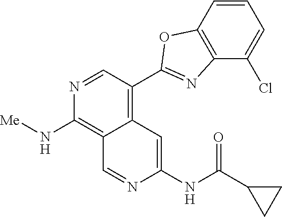 [SMILES: CNc1ncc(-c2nc3c(Cl)cccc3o2)c2cc(NC(=O)C3CC3)ncc12] | LCMS (ESI) m/z 394.1, [M + H]⁺. ¹H NMR (400 MHz, DMSO-d₆) δ 11.03 (s, 1H), 9.64 (s, 1H), 9.45 (s, 1H), 8.93 (s, 1H), 8.75-8.72 (m, 1H), 7.73 (d, J = 8.0 Hz, 1H), 7.47 (d, J = 8.0 Hz, 1H), 7.43-7.38 (m, 1H), 3.10 (d, J = 4.4 Hz, 3H), 2.11- 2.08 (m, 1H), 0.89-0.84 (m, 4H). |
| | N-(5-(4-chlorobenzo[d]oxazol-2-yl)-8- | |
| | (methylamino)-2,7-naphthyridin-3- | |
| | yl)cyclopropanecarboxamide | |
| Example 205 | 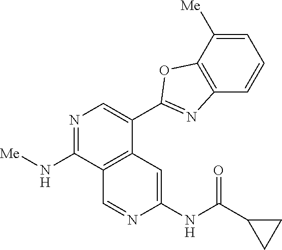 [SMILES: CNc1ncc(-c2nc3cccc(C)c3o2)c2cc(NC(=O)C3CC3)ncc12] | LCMS (ESI) m/z 374.2, [M + H]⁺. ¹H NMR (400 MHz, DMSO-d₆) δ 11.04 (s, 1H), 9.68 (s, 1H), 9.44 (s, 1H), 8.98 (s, 1H), 8.76-8.47 (m, 1H), 7.56 (d, J = 8.0 Hz, 1H), 7.33- 7.25 (m, 1H), 7.20 (d, J = 7.6 Hz, 1H), 3.10 (d, J = 4.4 Hz, 3H), 2.59 (s, 3H), 2.19-2.03 (m, 1H), 0.97- 0.77 (m, 4H). |
| | N-(8-(methylamino)-5-(7- | |
| | methylbenzo[d]oxazol-2-yl)-2,7- | |
| | naphthyridin-3- | |
| | yl)cyclopropanecarboxamide | |
| Example 206 | 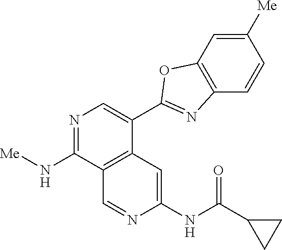 [SMILES: CNc1ncc(-c2nc3ccc(C)cc3o2)c2cc(NC(=O)C3CC3)ncc12] | LCMS (ESI) m/z 374.2, [M + H]⁺. ¹H NMR (400 MHz, DMSO-d₆) δ 11.02 (s, 1H), 9.65 (s, 1H), 9.43 (s, 1H), 8.89 (s, 1H), 8.63-8.60 (m, 1H), 7.62 (d, J = 8.4 Hz, 1H), 7.54 (s, 1H), 7.20 (d, J = 8.4 Hz, 1H), 3.09 (d, J = 4.4 Hz, 3H), 2.48 (s, 3H), 2.13-2.07 (m, 1H), 0.90- 0.85 (m, 4H). |
| | N-(8-(methylamino)-5-(6- | |
| | methylbenzo[d]oxazol-2-yl)-2,7- | |
| | naphthyridin-3- | |
| | yl)cyclopropanecarboxamide | |
| Example 207 | 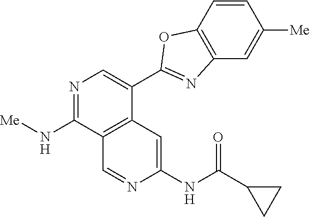 [SMILES: CNc1ncc(-c2nc3cc(C)ccc3o2)c2cc(NC(=O)C3CC3)ncc12] | LCMS (ESI) m/z 374.2, [M + H]⁺. ¹H NMR (400 MHz, DMSO-d₆) δ 11.03 (s, 1H), 9.69 (s, 1H), 9.43 (s, 1H), 8.89 (s, 1H), 8.67-8.59 (m, 1H), 7.63-7.53 (m, 2H), 7.23- 7.15 (m, 1H), 3.09 (d, J = 4.4 Hz, 3H), 2.46 (s, 3H), 2.15-2.04 (m, 1H), 0.93-0.88 (m, 2H), 0.88- 0.82 (m, 2H). |
| | N-(8-(methylamino)-5-(5- | |
| | methylbenzo[d]oxazol-2-yl)-2,7- | |
| | naphthyridin-3- | |
| | yl)cyclopropanecarboxamide | |

### Example 208: Synthesis of N-(5-(7-methoxybenzo[d]oxazol-2-yl)-8-(methylamino)-2,7-naphthyridin-3-yl)cyclopropanecarboxamide

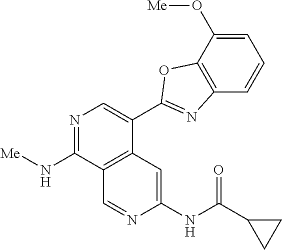 [SMILES: CNc1ncc(-c2nc3cccc(OC)c3o2)c2cc(NC(=O)C3CC3)ncc12]

### Step 1: 2-amino-6-methoxyphenol

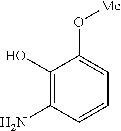

To a solution of 2-methoxy-6-nitrophenol (500.0 mg; 2.956 mmol; 1.00 eq.) in MeOH (10 mL) was added 10% Pd/C (150.0 mg; 30% w/w) under nitrogen atmosphere. The mixture was hydrogenated at 40 DC for 2 hours under hydrogen atmosphere (2 atm). After filtering through a celite pad, the filtrate was concentrated under reduced pressure. The residue was purified by flash chromatography on a silica gel column using 10-40% of EtOAc in petroleum ether as eluent to provide 2-amino-6-methoxyphenol as an off-white solid (392.0 mg, 89.5%), LCMS (ESI) m/z 140.1, [M+H]⁺.

### Step 2: 7-methoxybenzo[d]oxazole-2(3H)-thione

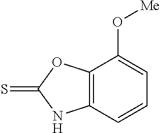 [SMILES: COc1cccc2[nH]c(=S)oc12]

7-methoxybenzo[d]oxazole-2(3H)-thione was synthesized using a similar procedure that was previously described in Example 202 by using 2-amino-6-methoxyphenol and potassium O-ethyl carbonodithioate as the starting material. LCMS (ESI) m/z 180.2, [M+H]⁺.

### Step 3: 2-chloro-7-methoxybenzo[d]oxazole

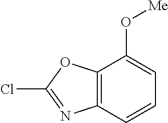 [SMILES: COc1cccc2nc(Cl)oc12]

2-chloro-7-methoxybenzo[d]oxazole was synthesized using a similar procedure that was previously described in Example 202, by using 7-methoxybenzo[d]oxazole-2(3H)-thione as the starting material. LCMS (ESI) m/z 184.0, [M+H]⁺.

### Step 4: N-(5-(7-methoxybenzo[d]oxazol-2-yl)-8-(methylamino)-2,7-naphthyridin-3-yl)cyclopropanecarboxamide

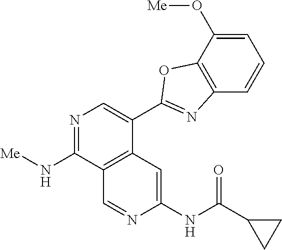 [SMILES: CNc1ncc(-c2nc3cccc(OC)c3o2)c2cc(NC(=O)C3CC3)ncc12]

N-(5-(7-methoxybenzo[d]oxazol-2-yl)-8-(methylamino)-2,7-naphthyridin-3-yl)cyclopropanecarboxamide was synthesized using a similar procedure that was previously described in Example 195 by using N-(8-(methylamino)-5-(4,4,5,5-tetramethyl-1,3,2-dioxaborolan-2-yl)-2,7-naphthyridin-3-yl)cyclopropanecarboxamide and 2-chloro-7-methoxybenzo[d]oxazole as the starting material. LCMS (ESI) m/z 390.1, [M+H]⁺. ¹H NMR (400 MHz, DMSO-d₆) δ 11.03 (s, 1H), 9.65 (s, 1H), 9.44 (s, 1H), 8.88 (s, 1H), 8.69-8.60 (m, 1H), 7.36 7.26 (m, 2H), 7.06-6.99 (m, 1H), 4.03 (s, 3H), 3.09 (d, 4.4 HZ, 3H), 2.15-2.04 (m, 1H), 0.97-0.79 (m, 4H).

### Example 209: Synthesis of N-(5-(5-bromobenzo[d]oxazol-2-yl)-8-(methylamino)-2,7-naphthyridin-3-yl)cyclopropanecarboxamide

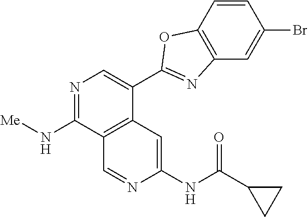 [SMILES: CNc1ncc(-c2nc3cc(Br)ccc3o2)c2cc(NC(=O)C3CC3)ncc12]

### Step 1: N-(5-(hydroxymethyl)-8-(methylamino)-2,7-naphthyridin-3-yl)cyclopropanecarboxamide

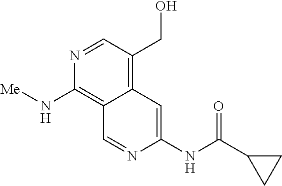 [SMILES: CNc1ncc(CO)c2cc(NC(=O)C3CC3)ncc12]

To a solution of N-(5-bromo-8-(methylamino)-2,7-naphthyridin-3-yl)cyclopropanecarboxamide (20.1 g; 62.271 mmol; 1.00 eq.) and Pd(PPh₃)₄ (14.4 g; 12.461 mmol; 020 eq.) in 1,4-dioxane (1 L) was added (tributylstannyl)methanol (60 g; 186.862 mmol; 3.00 eq.) under nitrogen atmosphere. The resulting solution was stirred for at 9° C. for 12 hours. The desired product was detected via LCMS. The reaction was concentrated under vacuum. The residue was purified by flash chromatography on silica gel column using 10-15% of MeOH in CH₂Cl₂ as eluent to provide N-(5-(hydroxymethyl)-8-(methylamino)-2,7-naphthyridin-3-yl)cyclopropanecarboxamide as a yellow solid (12 g, 70.8%), LCMS (ESI) m/z 273.1, [M+H]⁺.

### Step 2: N-(5-formyl-8-(methylamino)-2,7-naphthyridin-3-yl) cyclopropanecarboxamide

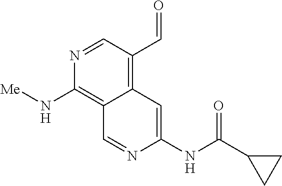 [SMILES: CNc1ncc(C=O)c2cc(NC(=O)C3CC3)ncc12]

A solution of N-[5-(hydroxymethyl)-8-(methylamino)-2,7-naphthyridin-3-yl]cyclopropanecarboxamide (12.05 g; 44.068 mmol; 1.00 eq.) in DMSO (240 mL) was heated until all of solids were dissolved. To this mixture were added CH₂Cl₂ (1200 mL) and pyridine (7 g; 88.496 mmol; 2.01 eq.) and Dess-Martin (20 g; 47.154 mmol; 1.07 eq.). The resulting solution was stirred at room temperature for 2 hours. Upon completion; the reaction mixture was filtered, and the filter cake was washed by CH₂Cl₂/MeOH. The filtrate was collected and concentrated under reduced pressure. The resulting mixture was diluted with EtOAc (1 L) and washed by brine (3×400 mL). The organic layer was dried over anhydrous sodium sulfate, filtered, and concentrated under vacuum until a large amount of precipitated product formed. The precipitated product were collected by filtration and washed with EtOAc to afford the crude product. This crude product was slurried in MeOH for 4 hours to provide N-(5-formyl-8-(methylamino)-2,7-naphthyridin-3-yl)cyclopropanecarboxamide as an off-white solid (6.5 g, 50.2%). LCMS (ESI) m/z 271.1, [M+H]⁺. ¹H NMR. (400 MHz, DMSO-d₆) δ 11.02 (s, 1H), 9.84 (s, 1H), 9.48 (s, 1H), 9.39 (s, 1H), 8.98-8.91 (m, 1H), 8.55 (s, 1H), 3.10 (d, J=4.4 Hz, 3H), 2.11-2.03 (m, 1H), 0.92-0.81 (m, 4H).

### Step 3: N-(5-(5-bromobenzo[d]oxazol-2-yl)-8-(methylamino)-2,7-naphthyridin-3-yl)cyclopropanecarboxamide

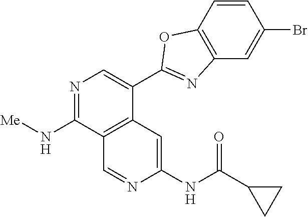 [SMILES: CNc1ncc(-c2nc3cc(Br)ccc3o2)c2cc(NC(=O)C3CC3)ncc12]

A solution of N-(5-formyl-8-(methylamino)-2,7-naphthyridin-3-yl)cyclopropanecarboxamide (1.00 g; 3.70 mmol; 1.00 eq.) and 2-amino-4-bromophenol (0.70 g; 3.72 mmol; 1.00 eq.) in toluene (40 mL) was heated at 110° C. for 16 hours. The mixture was concentrated under reduced pressure. The residue was dissolved by CH₂Cl₂ (40 mL), to this mixture was added DDQ (0.92 g; 4.05 mmol; 1.10 eq.). The resulting solution was stirred at room temperature for 2 hours. The solvent was concentrated under vacuum. The residue was purified by flash chromatography on pre-packed C18 column using 30-100% of MeOH in water (10 mmol/L NH₄HCO₃) as eluent to provide the crude product. The crude product was purified by flash chromatography on silica gel column using 0-20% of MeOH in CH₂Cl₂ as eluent to provide N-(5-(5-bromobenzo[d]oxazol-2-yl)-8-(methylamino)-2,7-naphthyridin-3-yl)cyclopropanecarboxamide as a yellow solid (520.0 mg, 32.2%). LCMS (ESI) m/z 438.0, [M+H]⁺. ¹H NMR (400 MHz, DMSO-d₆) δ11.05 (5, 1H), 9.64 (s, 1H), 9.44 (s, 1H), 8.93 (s, 1H), 8.82-8.59 (m, 1H), 7.95 (d, 1.6 Hz, 1H), 7.72 (d, J=8.8 Hz, 1H), 7.60-7.37 (m, 1H), 3.10 (d, J=4.4 Hz, 3H), 2.17-2.00 (m, 1H), 0.99-0.81 (m, 4H).

### Example 210: Synthesis of N-(5-(5-cyanobenzo[d]oxazol-2-yl)-8-(methylamino)-2,7-naphthyridin-3-yl)cyclopropanecarboxamide

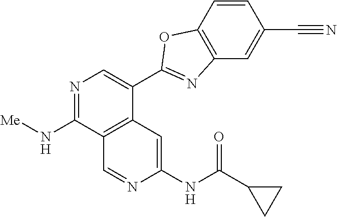 [SMILES: CNc1ncc(-c2nc3cc(C#N)ccc3o2)c2cc(NC(=O)C3CC3)ncc12]

A stirred solution of 3-amino-4-hydroxybenzonitrile (49.6 mg; 0.370 mmol; 1.00 eq.) and N-(5-formyl-8-(methylamino)-2,7-naphthyridin-3-yl)cyclopropanecarboxamide (100.0 mg; 0.370 mmol; 1.00 eq.) in a mixture solvent of DMSO/toluene (1:5, 12 mL) was stirred at 110° C. overnight. The resulting mixture was concentrated under reduced pressure to remove toluene. The resulting solution was diluted with CH₂Cl₂ (15 mL). To the mixture was added DDC). (920.4 mg; 0.407 mmol; 1.10 eq.). The reaction mixture was stirred at room temperature for 2 hours. The desired product was detected via LCMS. The resulting mixture was concentrated under reduced pressure. The residue was purified by flash chromatography on pre-packed C18 column using 20-80% of MeOH in water (10 mmol/L NH₄HCO₃) as eluent to provide the crude product. The crude product was dissolved in DMSO (2 mL) and dropwise into water (100 mL) and precipitate solids were formed. The precipitated solids were collected by filtration and washed with water (10 mL) to provide N-(5-(5-cyanobenzo[d]oxazol-2-yl)-8-(methylamino)-2,7-naphthyridin-3-yl)cyclopropanecarboxamide as a grey solid (63.9 mg, 43.4%). LCMS (ESI) m/z 385.1, [M+H]⁺. ¹H NMR (400 MHz, DMSO-d₆) δ 11.06 (s, 1H), 9.64 (s, 1H), 9.45 (s, 1H), 8.96 (s, 1H), 8.80-8.75 (m, 1H), 8.29 (d, 2.0 Hz, 1H), 7.94 (d, J=8.4 Hz, 1H), 7.85 (dd, 8.4, 2.0 Hz, 1H), 3.11 (d, J=4.4 Hz, 3H), 2.14-2.05 (m, 1H), 0.95-0.81 (m, 4H).

Each compound in Table 11 below was prepared using a similar experimental procedure to prepare Example 210, using N-(5-formyl-8-(methyl amino)-2,7-naphthyridin-3-yl)cyclopropanecarboxamide as the common intermediate and appropriate aminophenol.

TABLE 11

| Example | Structure and Name | MS and ¹H NMR |
|---|---|---|
| Example 211 | 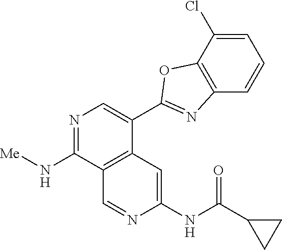 [SMILES: CNc1ncc(-c2nc3cccc(Cl)c3o2)c2cc(NC(=O)C3CC3)ncc12] | LCMS (ESI) m/z 394.1, [M + H]⁺. ¹H NMR (400 MHz, DMSO-d₆) δ 11.05 (s, 1H), 9.66 (s, 1H), 9.45 (s, 1H), 8.94 (s, 1H), 8.82-8.61 (m, 1H), 7.74 (d, J = 8.0 Hz, 1H), 7.49 (d, J = 8.0 Hz, 1H), 7.44-7.26 (m, 1H), 3.10 (d, J = 4.4 Hz, 3H), 2.19- 1.98 (m, 1H), 0.99-0.82 (m, 4H). |
| | N-(5-(7-chlorobenzo[d]oxazol-2-yl)-8- | |
| | (methylamino)-2,7-naphthyridin-3- | |
| | yl)cyclopropanecarboxamide | |
| Example 212 | 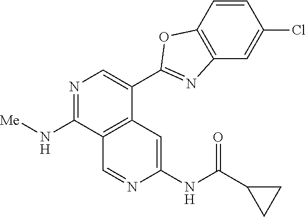 [SMILES: CNc1ncc(-c2nc3cc(Cl)ccc3o2)c2cc(NC(=O)C3CC3)ncc12] | LCMS (ESI) m/z 394.1, [M + H]⁺. ¹H NMR (400 MHz, DMSO-d₆) δ 11.04 (s, 1H), 9.63 (s, 1H), 9.44 (s, 1H), 8.93 (s, 1H), 8.8-8.59 (m, 1H), 7.82 (d, J = 2.0 Hz, 1H), 7.77 (d, J = 8.8 Hz, 1H), 7.52-7.18 (m, 1H), 3.10 (d, J = 4.4 Hz, 3H), 2.17- 2.00 (m, 1H), 1.00-0.72 (m, 4H). |
| | N-(5-(5-chlorobenzo[d]oxazol-2-yl)-8- | |
| | (methylamino)-2,7-naphthyridin-3- | |
| | yl)cyclopropanecarboxamide | |
| Example 213 | 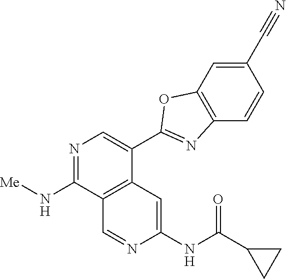 [SMILES: CNc1ncc(-c2nc3ccc(C#N)cc3o2)c2cc(NC(=O)C3CC3)ncc12] | LCMS (ESI) m/z 385.1, [M + H]⁺. ¹H NMR (400 MHz, DMSO-d₆) δ 11.07 (s, 1H), 9.67 (s, 1H), 9.43 (s, 1H), 8.95 (s, 1H), 8.84-8.79 (m, 1H), 8.32 (d, J = 1.2 Hz, 1H), 7.90 (d, J = 8.4 Hz, 1H), 7.81 (dd, J = 8.4, 1.2 Hz, 1H), 3.10 (d, J = 4.4 Hz, 3H), 2.14-2.06 (m, 1H), 0.94- 0.83 (m, 4H). |
| | N-(5-(6-cyanobenzo[d]oxazol-2-yl)-8- | |
| | (methylamino)-2,7-naphthyridin-3- | |
| | yl)cyclopropanecarboxamide | |
| Example 214 | 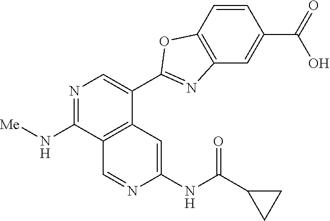 [SMILES: CNc1ncc(-c2nc3cc(C(=O)O)ccc3o2)c2cc(NC(=O)C3CC3)ncc12] | LCMS (ESI) m/z 404.1, [M + H]⁺. |
| | 2-(6-(cyclopropanecarboxamido)-1- | |
| | (methylamino)-2,7-naphthyridin-4- | |
| | yl)benzo[d]oxazole-5-carboxylic acid | |
| Example 215 | 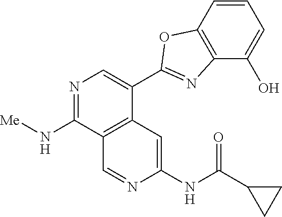 [SMILES: CNc1ncc(-c2nc3c(O)cccc3o2)c2cc(NC(=O)C3CC3)ncc12] | LCMS (ESI) m/z 376.1, [M + H]⁺. ¹H NMR (400 MHz, DMSO-d₆) δ 11.02 (s, 1H), 10.21 (s, 1H), 9.45 (s, 1H), 9.44 (s, 1H), 8.83 (s, 1H), 8.61-8.57 (m, 1H), 7.17-7.11 (m, 2H), 6.81-6.76 (m, 1H), 3.08 (d, J = 4.4 Hz, 3H), 2.11-2.04 (m, 1H), 0.92-0.80 (m, 4H). |
| | N-(5-(4-hydroxybenzo[d]oxazol-2-yl)- | |
| | 8-(methylamino)-2,7-naphthyridin-3- | |
| | yl)cyclopropanecarboxamide | |
| Example 216 | 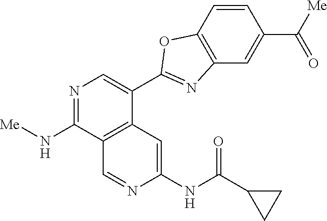 [SMILES: CNc1ncc(-c2nc3cc(C(C)=O)ccc3o2)c2cc(NC(=O)C3CC3)ncc12] | LCMS (ESI) m/z 402.1, [M + H]⁺. ¹H NMR (400 MHz, DMSO-d₆) δ 11.04 (s, 1H), 9.72 (s, 1H), 9.46 (s, 1H), 8.97 (s, 1H), 8.78-8.68 (m, 1H), 8.33 (d, J = 1.6 Hz, 1H), 8.03 (dd, J = 8.4, 1.6 Hz, 1H), 7.85 (d, J = 8.4 Hz, 1H), 3.11 (d, J = 4.4 Hz, 3H), 2.71 (s, 3H), 2.16-2.07 (m, 1H), 0.97-0.91 (m, 2H), 0.90- 0.85 (m, 2H). |
| | N-(5-(5-acetylbenzo[d]oxazol-2-yl)-8- | |
| | (methylamino)-2,7-naphthyridin-3- | |
| | yl)cyclopropanecarboxamide | |
| Example 217 | 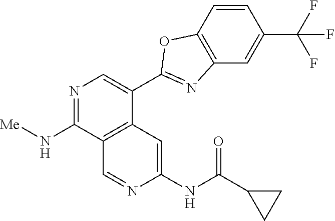 [SMILES: CNc1ncc(-c2nc3cc(C(F)(F)F)ccc3o2)c2cc(NC(=O)C3CC3)ncc12] | LCMS (ESI) m/z 428, [M + H]⁺. ¹H NMR (400 MHz, DMSO-d₆) δ 11.06 (s, 1H), 9.69 (s, 1H), 9.46 (s, 1H), 8.98 (s, 1H), 8.80-8.72 (m, 1H), 8.10 (d, J = 2.4 Hz, 1H), 7.97 (d, J = 8.4 Hz, 1H), 7.80-7.75 (m, 1H), 3.11 (d, J = 4.4 Hz, 3H), 2.15- 2.05 (m, 1H), 0.96-0.89 (m, 2H), 0.90-0.82 (m, 2H). |
| | N-(8-(methylamino)-5-(5- | |
| | (trifluoromethyl)benzo[d]oxazol-2- | |
| | yl)-2,7-naphthyridin-3- | |
| | yl)cyclopropanecarboxamide | |
| Example 218 | 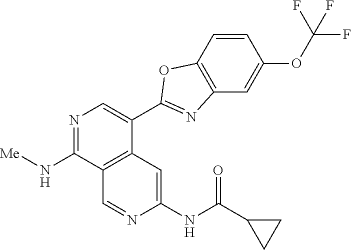 [SMILES: CNc1ncc(-c2nc3cc(OC(F)(F)F)ccc3o2)c2cc(NC(=O)C3CC3)ncc12] | LCMS (ESI) m/z 444.1, [M + H]⁺. ¹H NMR (400 MHz, DMSO-d₆) δ 11.05 (s, 1H), 9.65 (s, 1H), 9.45 (s, 1H), 8.94 (s, 1H), 8.77-8.70 (m, 1H), 7.86 (d, J = 8.8 Hz, 1H), 7.78 (d, J = 2.4 Hz, 1H), 7.39 (dd, J = 8.8, 2.4 Hz, 1H), 3.10 (d, J = 4.4 Hz, 3H), 2.16-2.05 (m, 1H), 0.96- 0.82 (m, 4H). |
| | N-(8-(methylamino)-5-(5- | |
| | (trifluoromethoxy)benzo[d]oxazol-2- | |
| | yl)-2,7-naphthyridin-3- | |
| | yl)cyclopropanecarboxamide | |
| Example 219 | 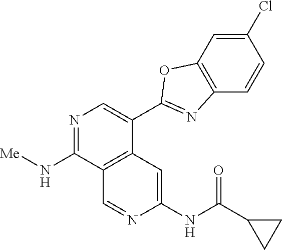 [SMILES: CNc1ncc(-c2nc3ccc(Cl)cc3o2)c2cc(NC(=O)C3CC3)ncc12] | LCMS (ESI) m/z 394.1, [M + H]⁺. ¹H NMR (400 MHz, DMSO-d₆) δ 11.04 (s, 1H), 9.63 (s, 1H), 9.44 (s, 1H), 8.92 (s, 1H), 8.74-8.69 (m, 1H), 7.82 (d, J = 2.0 Hz, 1H), 7.76 (d, J = 8.4 Hz, 1H), 7.43-7.40 (m, 1H), 3.09 (d, J = 4.4 Hz, 3H), 2.15- 2.07 (m, 1H), 0.95-0.84 (m, 4H). |
| | N-(5-(6-chlorobenzo[d]oxazol-2-yl)-8- | |
| | (methylamino)-2,7-naphthyridin-3- | |
| | yl)cyclopropanecarboxamide | |

### Example 220: Synthesis of N-(5-(4-(2-(methoxymethyl)azetidin-1-yl)benzo[d]oxazol-2-yl)-8-(methylamino)-2,7-naphthyridin-3-yl)cyclopropanecarboxamide

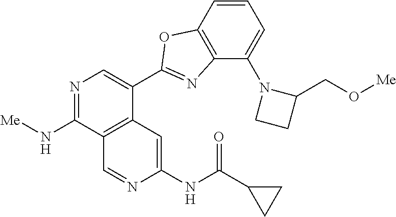 [SMILES: CNc1ncc(-c2nc3c(N4CCC4COC)cccc3o2)c2cc(NC(=O)C3CC3)ncc12]

### Step 1: tert-butyl 2-(methoxymethyl)azetidine-1-carboxylate

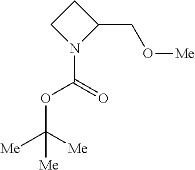 [SMILES: COCC1CCN1C(=O)OC(C)(C)C]

To a stirred solution of tert-butyl 2-(hydroxymethyl)azetidine-1-carboxylate (6.00 g; 32.045 mmol; 1.00 eq.) in THF (20 mL) was added NaH (60%) (1.55 g; 38.753 mmol; 1.21 eq.) at 0° C. under nitrogen atmosphere. The resulting mixture was stirred at room temperature for 0.5 hour. To the above solution was added iodomethane (6.80 g; 47.908 mmol; 1.50 eq.) dropwise at room temperature. The resulting mixture was stirred at room temperature for 1 hour under nitrogen atmosphere. LCMS indicated that the starting material was consumed. The reaction was quenched with a saturated NH₄Cl aqueous solution (100 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by flash chromatography on silica gel column using 0-30% of EtOAc in petroleum ether as eluent to provide tert-butyl 2-(methoxymethyl)azetidine-1-carboxylate as a light yellow oil (5.4 g, 83.7%). LCMS (ESI) m/z 202.1, [M+H]⁺.

### Step 2: 2-(methoxymethyl)azetidine hydrochloride

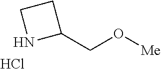

A solution of trot-butyl 2-(methoxymethyl)azetidine-1-carboxylate (1.70 g; 8.447 mmol, 1.00 eq.) and HCl(gas)(4 M in 1,4-dioxane, 3 mL) in MeOH (15 mL) was stirred at room temperature for 2 hours. The desired product was detected via LCMS. The resulting mixture was concentrated under reduced pressure to afford 2-(methoxymethyl)azetidine hydrochloride as a colorless oil (1.1 g, 94.6%). LCMS (ESI) m/z 102.2, [M+H]⁺.

### Step 3: 3-(2-(methoxymethyl)azetidin-1-yl)-2-nitrophenol

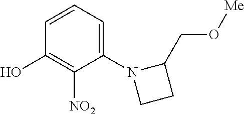 [SMILES: COCC1CCN1c1cccc(O)c1[N+](=O)[O-]]

To a stirred solution of 2-(methoxymethyl)azetidine hydrochloride (800.0 mg; 5.814 mmol; 1.00 eq.) and 3-fluoro-2-nitrophenol (1.00 g; 6.365 mmol; 1.09 eq.) in IMF (8 ml) was added KOH (163.0 mg; 2.905 mmol; 0.50 eq.) at room temperature. The resulting mixture was stirred at 90° C. for 2 hours. The desired product was detected via LCMS. The mixture was allowed to cool down to room temperature. The resulting mixture was diluted with brine (50 mL) and extracted with EtOAc (2×30 mL). The combined organic layers were washed with brine (5×20 mL), dried over anhydrous Na₂SO₄, filtered, and concentrated under reduced pressure. The residue was purified by flash chromatography on silica gel column using 10-50% of CH₂Cl₂ in petroleum ether as eluent to provide 3-(2-(methoxymethyl)azetidin-1-yl)-2-nitrophenol as a red solid (462.0 mg, 33.4%). LCMS (ESI) m/z 239.1, [M+H]⁺.

### Step 4: 2-amino-3-(2-(methoxymethyl)azetidin-1-yl)phenol

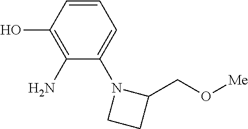 [SMILES: COCC1CCN1c1cccc(O)c1N]

To a solution of 3-(2-(methoxymethyl)azetidin-1-yl)-2-nitrophenol (462.0 mg; 1.939 mmol; 1.00 eq.) in WIRE (10 mL) was added 10% Pd/C (1.00 g; 100% w/w) under nitrogen atmosphere. The mixture was hydrogenated at room temperature for 14 hours under hydrogen atmosphere (2 atm). After filtration, the filtrate was concentrated under reduced pressure to afford 2-amino-3-(2-(methoxymethyl)azetidin-1-yl)phenol as a brown solid (320.0 mg, 79.2%), LCMS (ESI) m/z 209.1, [M+H]⁺.

### Step 5: N-(5-(4-(2-(methoxymethyl)azetidin-1-yl)benzo[d]oxazol-2-yl)-8-(methylamino)-2,7-naphthyridin-3-yl)cyclopropanecarboxamide

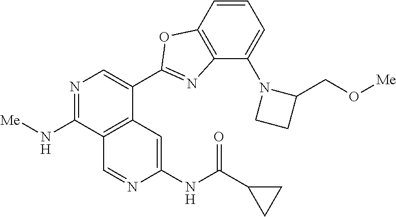 [SMILES: CNc1ncc(-c2nc3c(N4CCC4COC)cccc3o2)c2cc(NC(=O)C3CC3)ncc12]

A solution of 2-amino-3-(2-(methoxymethyl)azetidin-1-yl)phenol (30.8 mg; 0.148 mmol; 1.00 eq.) and N-(5-formyl-8-(methylamino)-2,7-naphthyridin-3-yl)cyclopropanecarboxamide (40.0 mg; 0.148 mmol; 1.00 eq.) in toluene (3 mL) was stirred at 110° C. for 24 hours. The desired product was detected via LCMS. The mixture was concentrated under reduced pressure. The residue was purified by flash chromatography on pre-packed. C18 column using 35-100% of MeCN in water (10 mmol/L NH₄HCO₃) to provide a crude product. The crude product was further purified by reverse phase preparative HPLC (XBridge Prep OBD C18 Column, 5 μm, 30×250 mm, waters; gradient elution of 55-63% MeCN in water over a 9 min period, where water contains 10 mmol/L NH₄HCO₃, flow rate: 60 mL/min, detector UV wavelength: 254 nm) to afford N-(5-(4-(2-(methoxymethyl)azetidin-1-yl)benzo[d]oxazol-2-yl)-8-(methylamino)-2,7-naphthyridin-3-yl)cyclopropanecarboxamide as a dark green solid (8.1 mg, 11.9%). LCMS (ESI) m/z 459.2, [M+H]⁺.

### Example 221: Synthesis of N-(5-(4-(3-methoxyazetidin-1-yl)benzo[d]oxazol-2-yl)-8-(methylamino)-2,7-naphthyridin-3-yl)cyclopropanecarboxamide

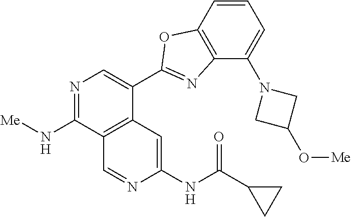 [SMILES: CNc1ncc(-c2nc3c(N4CC(OC)C4)cccc3o2)c2cc(NC(=O)C3CC3)ncc12]

### Step 1: 3-(3-methoxyazetidin-1-yl-2-nitrophenol

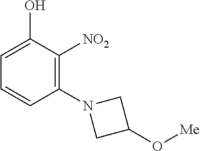 [SMILES: COC1CN(c2cccc(O)c2[N+](=O)[O-])C1]

To a stirred solution of 3-fluoro-2-nitrophenol (1.56 g; 9.930 mmol; 1.00 eq.) and 3-methoxyazetidine hydrochloride (2.46 g; 19.906 mmol; 2.00 eq.) in DMF (20 mL) was added KOH (300.0 mg; 5.347 mmol; 0.54 eq.). The resulting mixture was stirred at 90° C. for 2 hours. The desired product was detected via LCMS. The resulting mixture was diluted with CH₂Cl₂ (80 mL) and washed with water (4×20 ML). The organic layer was dried over anhydrous Na₂SO₄. After filtration, the filtrate was concentrated under reduced pressure. The residue was purified by flash chromatography on silica gel column using 90% of petroleum ether in CH₂Cl₂ as eluent to provide 3-(3-methoxyazetidin-1-yl)-2-nitrophenol as a red oil (1.0 g, 45.4%). LCMS (ESI) m/z 225.1.

### Step 2: 2-amino-3-(3-methoxyazetidin-1-yl)phenol

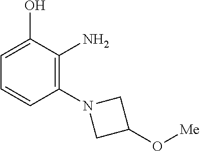 [SMILES: COC1CN(c2cccc(O)c2N)C1]

To a solution of 3-(3-methoxyazetidin-1-yl)-2-nitrophenol (400 mg; 1.784 mmol; 1 eq.) in MeOH (6 mL) was added 10% Pd/C (80.0 mg; 25% w/w) under nitrogen atmosphere. The mixture was hydrogenated at room temperature for 14 hours under hydrogen atmosphere (2 atm). The desired product was detected via. LCMS. The resulting mixture was filtered, the filter cake was washed with MeOH (2×2 mL). The filtrate was collected and concentrated under reduced pressure to afford 2-amino-3-(3-methoxyazetidin-1-yl)phenol as a black solid (320.0 mg, 92.4%). LCMS (ESI) m/z 195.1, [M+H]⁺.

### Step 3: N-(5-(4-(3-methoxyazetidin-1-yl)benzo[d]oxazol-2-yl)-8-(methylamino)-2,7-naphthyridin-3-yl)cyclopropanecarboxamide

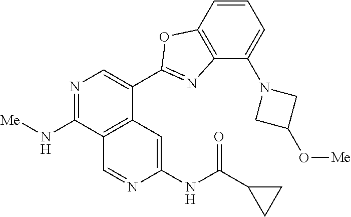 [SMILES: CNc1ncc(-c2nc3c(N4CC(OC)C4)cccc3o2)c2cc(NC(=O)C3CC3)ncc12]

A solution of N-(5-formyl-8-(methylamino)-2,7-naphthyridin-3-yl)cyclopropanecarboxamide (40.0 mg; 0.148 mmol; 1.00 eq.) and 2-amino-3-(3-methoxyazetidin-1-yl)phenol (28.7 mg; 0.148 mmol; 1.00 eq.) in toluene (3 mL) was stirred at 110° C. for 16 hours. The desired product was detected via LCMS. The mixture was concentrated under reduced pressure. The residue was purified by flash chromatography on pre-packed. C18 column using 5-100% of MeCN in water (10 mmol/L NH₄HCO₃) to provide a crude product. The crude product was purified by reverse phase preparative HPLC (Column: XBridge Prep OBD C18 Column, 5 μm, 30×150 mm, waters; gradient elution of 52-60% MeCN in water over a 11 min period, where water contains 10 mmol/L NH₄HCO₃, flow rate: 60 mL/min, detector UV wavelength: 254 nm) to afford N-(5-(4-(3-methoxyazetidin-1-yl)benzo[d]oxazol-2-yl)-8-(methylamino)-2,7-naphthyridin-3-yl)cyclopropanecarboxamide as a dark green solid (11.3 mg, 17.1%). LCMS (ESI) m/z 445.2, [M+H]⁺. ¹H NMR (400 MHz, DMSO-d₆) δ 11.02 (s, 1H), 9.83 (s, 1H), 9.43 (5, 1H), 8.88 (s, 1H), 8.68-8.78 (m, 1H), 7.27-7.10 (m, 1H), 6.97 (d, J=8.0, 1H), 6.27 (d, J=8.0, 1H), 4.67-4.49 (m, 2H), 4.46-4.30 (m, 1H), 4.18-3.89 (m, 2H), 3.33 (s, 3H), 3.09 (d, J=4.4 Hz, 3H), 2.17-2.05 (m, 1H), 0.96-0.75 (m, 4H).

### Example 222: Synthesis of N-(5-(4-cyclopropoxybenzo[d]oxazol-2-yl)-8-(methylamino)-2,7-naphthyridin-3-yl)cyclopropanecarboxamide

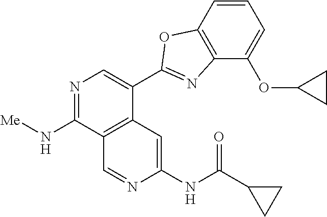 [SMILES: CNc1ncc(-c2nc3c(OC4CC4)cccc3o2)c2cc(NC(=O)C3CC3)ncc12]

### Step 1: 1-(benzyloxy)-3-fluoro-2-nitrobenzene

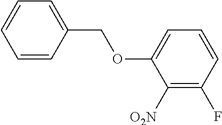 [SMILES: O=[N+]([O-])c1c(F)cccc1OCc1ccccc1]

To a stirred solution of 3-fluoro-2-nitrophenol (1.00 g; 6.365 mmol; 1.00 eq.) in acetone (40 mL) were added K₂CO₃ (2.64 g; 19.095 mmol; 3.00 eq.) and benzyl bromide (1.63 g; 9.547 mmol; 1.5 eq.) at 0° C. under nitrogen atmosphere. The mixture was stirred at 60° C. for 14 hours. The mixture was filtered, the filtrate was concentrated under reduced pressure. The residue was purified by flash chromatography on silica gel column using 0-30% of EtOAc in petroleum ether as eluent to provide 1-(benzyloxy)-3-fluoro-2-nitrobenzene as a colorless oil (1.3 g, 82.6%). ¹H NMR (300 MHz; DMSO-d₆) δ 7.72-7.55 (m, 1H); 7.47-7.08 (m, 7H), 5.34 (s, 2H).

### Step 2: 1-(benzyloxy)-3-cyclopropoxy-2-nitrobenzene

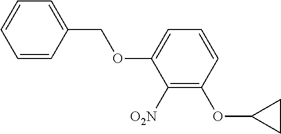 [SMILES: O=[N+]([O-])c1c(OCc2ccccc2)cccc1OC1CC1]

To a stirred solution of cyclopropanol (185.7 mg; 0.3201 mmol; 1.00 eq.) in THF (24 mL) was added NaH (60%) (1.28 g; 31.956 mmol; 10.00 eq.) at 0° C. under nitrogen atmosphere. The resulting solution was stirred at room temperature for 0.5 hour. To the mixture was added 1-(benzyloxy)-3-fluoro-2-nitrobenzidine (790.0 mg; 3.192 mmol; 1.00 eq.). The mixture was stirred at room temperature for 14 hours. The mixture was quenched with a saturated NH₄Cl solution (10 mL), extracted with EtOAc (3×50 mL). The combined organic layers were washed with brine (3×10 mL), dried over anhydrous Na₂SO₄. After filtration, the filtrate was concentrated under reduced pressure. The residue was purified by flash chromatography on silica gel column using 0-20% of EtOAc in petroleum ether as eluent to provide 1-(benzyloxy)-3-cyclopropoxy-2-nitrobenzene as a white solid (200.0 mg, 22.0%). ¹H NMR (300 MHz, DMSO-d₆) δ 7.57-7.26 (m, 6H), 7.13 (dd, J=8.6, 1.2 Hz, 1H), 7.00 (dd, J=8.6, 1.2 Hz, 1H), 5.25 (s, 2H), 4.13-3.95 (m, 1H), 0.90-0.57 (m, 4H).

### Step 3: 2-amino-3-cyclopropoxyphenol

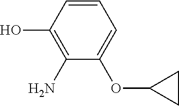 [SMILES: Nc1c(O)cccc1OC1CC1]

To a stirred solution of 1-(benzyloxy)-3-cyclopropoxy-2-nitrobenzene (100.0 mg; 0.351 mmol, 1.00 eq.) in MeOH, (12 mL) was added 10% Pd/C (35.0 mg; 35% w/w) under nitrogen atmosphere. The mixture was hydrogenated at room temperature for 2 hours under hydrogen atmosphere (2 atm). After filtration, the filtrate was concentrated under vacuum to afford 2-amino-3-cyclopropoxyphenol as a colorless crude oil (49.0 mg, 84.6%). LCMS (ESI) m/z 1662, [M+H]⁺.

### Step 4: N-(5-(4-cyclopropoxybenzo[d]oxazol-2-yl)-8-(methylamino)-2,7-naphthyridin-3-yl)cyclopropanecarboxamide

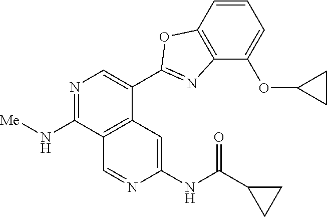 [SMILES: CNc1ncc(-c2nc3c(OC4CC4)cccc3o2)c2cc(NC(=O)C3CC3)ncc12]

N-(5-(4-cyclopropoxybenzo[d]oxazol-2-yl)-8-(methyl amino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-3-cyclopropoxyphenol as the starting material. LCMS (ESI) m/z 416.2, [M+H]⁺. ¹H NMR (400 MHz, DMSO-d₆) δ 11.04 (s, 1H), 9.74 (s, 1H), 9.44 (s, 114), 8.90 (s, 1H), 8.70-8.49 (m, 1H), 7.42-7.20 (m, 2H), 7.01 (d, J=7.6 Hz, 1H), 4.91-4.63 (m, 1H), 3.10 (d, J=4.4 Hz, 3H), 2.17-1.96 (m, 1H), 0.89-0.72 (m, 8H).

### Example 223: Synthesis of N-(5-(5-cyclopropoxybenzo[d]oxazol-2-yl)-8-(methylamino)-2,7-naphthyridin-3-yl)cyclopropanecarboxamide

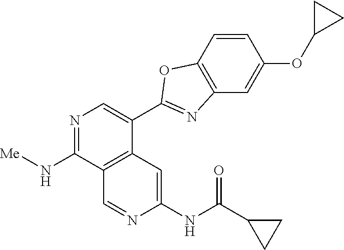 [SMILES: CNc1ncc(-c2nc3cc(OC4CC4)ccc3o2)c2cc(NC(=O)C3CC3)ncc12]

### Step 1: 1-(benzyloxy)-4-cyclopropoxybenzene

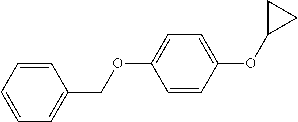 [SMILES: c1ccc(COc2ccc(OC3CC3)cc2)cc1]

To a solution of 4-(benzyloxy)phenol (2.00 g; 9.988=lot; 1.00 eq.) and Cs₂CO₃ (6.52 g; 20.011 mmol, 2.00 eq.) in DMF (10 mL) was added bromocyclopropane (3.63 g; 30.006 mmol; 3.00 eq.) via syringe. The resulting solution was stirred at 120° C. overnight. The desired product was detected via LCMS. The resulting mixture was diluted with EtOAc (200 mL) and washed with brine (5×30 mL). The organic phase was dried with Na₂SO₄ and concentrated under vacuum. The residue was purified by flash chromatography on silica gel column using 0-20% of EtOAc in petroleum ether as eluent to provide 1-(benzyloxy)-4-cyclopropoxybenzene as a red solid (1.30 g, 54.1%). LCMS (ESI) m/z 239.1, [M+H]⁺.

### Step 2: 4-cyclopropoxyphenol

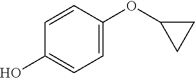 [SMILES: Oc1ccc(OC2CC2)cc1]

To a solution of 1-(benzyloxy)-4-cyclopropoxybenzene (1.30 g; 5.410 mmol; 1.00 eq.) in MeOH (40 mL) was added 10% Pd/C (390.0 mg; 30% w/w) under nitrogen atmosphere. The mixture was hydrogenated at 40° C. overnight under hydrogen atmosphere (2 atm). The desired product was detected via LCMS. The reaction was filtered, the filtrate was concentrated under vacuum. The residue was purified by flash chromatography on silica gel column using 0-20% of EtOAc in petroleum ether as eluent to provide 4-cyclopropoxyphenol as a white solid (560.0 mg, 68.9%). LCMS (ESI) m/z 149.1, [M+H]⁻.

### Step 3: 4-cyclopropoxy-2-nitrophenol

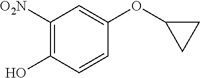 [SMILES: O=[N+]([O-])c1cc(OC2CC2)ccc1O]

NBS (546.0 mg; 3,068 mmol; 1.00 eq.) and AgNO₃ (521.0 mg; 3.067 mmol; 1.00 eq.) were mixed in MeCN (5 mL) at 70° C. The resulting white turbid solution was added dropwise to a solution of 4-cyclopropoxyphenol (460.0 mg; 3.063 mmol; 1.00 eq.) in MeCN (35 mL). The resulting mixture was stirred at 80° C. for 0.5 hour. The desired product was detected via LCMS. The resulting mixture was concentrated under vacuum. The residue was purified by flash chromatography on silica gel column using 0-20% of EtOAc in petroleum ether as eluent to provide 4-cyclopropoxy-2-nitrophenol as a yellow solid (236.0 mg, 39.4%). LCMS (ESI) m/z 194.1, [M+H]⁻.

### Step 4: 2-amino-4-cyclopropoxyphenol

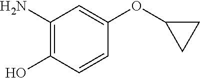 [SMILES: Nc1cc(OC2CC2)ccc1O]

To a solution of 4-cyclopropoxy-2-nitrophenol (100.0 mg; 0.512 mmol; 1.00 eq.) in MeOH (10 mL) was added 10% Pd/C (100.0 mg; 100% w/w) under nitrogen atmosphere. The mixture was hydrogenated at 40° C. overnight under hydrogen atmosphere (2 atm). The desired product was detected via LCMS. The resulting mixture was filtered, the filter cake was washed with MeOH (30 mL). The filtrate was concentrated under reduced pressure to provide 2-amino-4-cyclopropoxyphenol as a black solid (80.0 mg, 94.5%). LCMS (ESI) m/z 166.1, [M+H]⁺.

### Step 5: N-(5-(5-cyclopropoxybenzo[d]oxazol-2-yl)-8-(methylamino)-2,7-naphthyridin-3-yl)cyclopropanecarboxamide

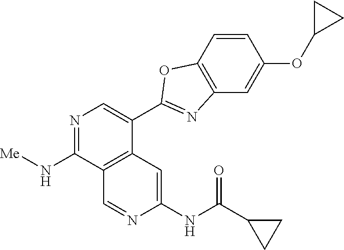 [SMILES: CNc1ncc(-c2nc3cc(OC4CC4)ccc3o2)c2cc(NC(=O)C3CC3)ncc12]

N-(5-(5-cyclopropoxybenzo[d]oxazol-2-yl)-8-(methylamino)-2,7-naphthyridin-3-yl)cyclopropanecarboxamide was synthesized using a similar procedure that was previously, described in Example 210, by using N-(5-formyl-8-(methylamino)-2,7-naphthyridin-3-yl)cyclopropanecarboxamide and 2-amino-4-cyclopropoxyphenol as the starting material. LCMS (ESI) m/z 416.2, [M+H]⁺. ¹H NMR (400 MHz, DMSO-d₆) δ 11.03 (s, 1H), 9.63 (s, 1H), 9.43 (s, 1H), 8.89 (s, 1H), 8.66-8.61 (m, 1H), 7.62 (d, J=8.8 Hz, 1H), 7.44 (d, J=2.4 Hz, 1H), 6.99 (dd, 8.8, 2.4 Hz, 1H), 4.01-3.93 (m, 1H), 3.09 (d, J=4.4 Hz, 3H), 2.15-2.05 (m, 1H), 0.96-0.83 (m, 6H), 0.76-0.66 (m, 2H).

### Example 224: Synthesis of N-(5-(4,5-dimethoxybenzo[d]oxazol-2-yl-8-(methylamino)-2,7-naphthyridin-3-yl)cyclopropanecarboxamide

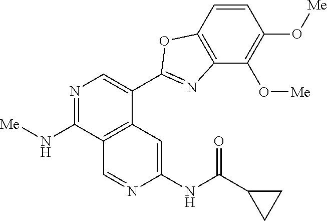 [SMILES: CNc1ncc(-c2nc3c(OC)c(OC)ccc3o2)c2cc(NC(=O)C3CC3)ncc12]

### Step 1: 3,4-dimethoxy-2-nitrobenzaldehyde

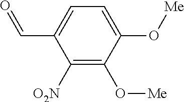 [SMILES: COc1ccc(C=O)c([N+](=O)[O-])c1OC]

A solution of 4-hydroxy-3-methoxy-2-nitrobenzaldehyde (1.18 g; 6.000 mmol; 1.00 eq.), K₂CO₃ (2.48 g; 18.001 mmol; 3.00 eq.) and methyl iodide (1.70 g; 12.000 mmol; 2.00 eq.) in DMF (12 mL) was stirred at room temperature for 14 hours under nitrogen atmosphere. The desired product was detected via LCMS. The resulting mixture was diluted with EtOAc (100 mL) and washed with brine 20 mL). The organic phase was dried over anhydrous Na₂SO₄ and concentrated under vacuum. The residue was purified by flash chromatography on silica gel column using 0-30% of EtOAc in petroleum ether as eluent to provide 3,4-dimethoxy-2-nitrobenzaldehyde as a yellow solid. (1.24 g, 97.9%). LCMS (ESI) ink 212.0, [M+H]⁺.

### Step 2: 3,4-dimethoxy-2-nitrophenol

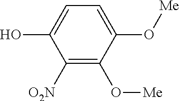 [SMILES: COc1ccc(O)c([N+](=O)[O-])c1OC]

To a stirred solution of 3,4-dimethoxy-2-nitrobenzaldehyde (527.9 mg; 2,500 mmol; 1.00 eq.) in CH₂Cl₂ (20 mL) were added m-CPBA (862.7 mg; 5.000 mmol; 2.00 eq.) and TFA (285.0 mg; 2.500 mmol, 1.00 eq.) at 0° C. under nitrogen atmosphere. The reaction was stirred at room temperature for 18 hours. The reaction was cooled to 0° C., excess reagent was quenched with 5% sodium hydrogen sulfite solution. The precipitated product were removed by filtration, washed with dichloromethane. The filtrate was washed with saturated sodium bicarbonate solution and brine, dried over anhydrous Na₂SO₄, filtered, and concentrated under vacuum to provide a yellow solid. This intermediate was suspended in methanol (9 mL), treated with 2 M NaOH (3 mL) at room temperature for 1 hour. The reaction was acidified with 1 N HCl. The solids were collected by filtration and washed with methanol to provide 3,4-dimethoxy-2-nitrophenol as a red solid (420.9 mg, 84.5%). LCMS (ESI) m/z 200.0, [M+H]⁺.

### Step 3: 2-amino-3,4-dimethoxyphenol

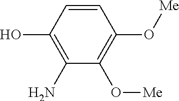 [SMILES: COc1ccc(O)c(N)c1OC]

To a solution of 3,4-dimethoxy-2-nitrophenol (420.0 mg; 2.109 mmol; 1.00 eq.) in MeOH (50 mL) was added 10% Pd/C (126.0 mg; 30% w/w) under nitrogen atmosphere. The mixture was hydrogenated at 40° C. for 2 hours under hydrogen atmosphere (2 atm). After being filtered through a celite pad and the filtrate was concentrated under reduced pressure to afford 2-amino-3,4-dimethoxyphenol as a brown-yellow solid (320.1 mg, 89.6%). LCMS (ESI) m/z 170.1, [M+H]⁺.

### Step 4: N-(5-(4,5-dimethoxybenzo[d]oxazol-2-yl)-8-(methylamino)-2,7-naphthyridin-3-yl)cyclopropanecarboxamide

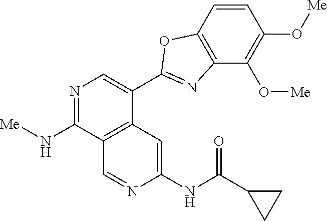 [SMILES: CNc1ncc(-c2nc3c(OC)c(OC)ccc3o2)c2cc(NC(=O)C3CC3)ncc12]

N-(5-(4,5-dimethoxybenzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-3,4-dimethoxyphenol as the starting material. LCMS (ESI) m/z 420.2. ¹H NMR (400 MHz, DMSO-d₆) δ 11.05 (s, 1H), 9.91 (s, 1H), 9.44 (s, 1H), 8.90 (s, 1H), 8.69-8.63 (m, 1H), 7.24 (d, =8.8 HZ, 1H), 7.04 (d, 8.8 Hz, 1H), 4.43 (s, 3H), 3.81 (s, 3R), 3.09 (d, J=4.4 Hz 3H), 2.12-2.04 (m, 1H), 0.90-0.82 (m, 4R).

### Example 225: Synthesis of N-(5-(5-(difluoromethoxy)benzo[d]oxazol-2-yl)-8-(methylamino)-2,7-naphthyridin-3-yl)cyclopropanecarboxamide

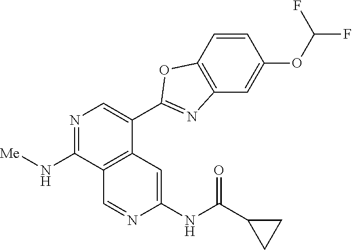 [SMILES: CNc1ncc(-c2nc3cc(OC(F)F)ccc3o2)c2cc(NC(=O)C3CC3)ncc12]

### Step 1: 1-(benzyloxy)-4-(difluoromethoxy)benzene

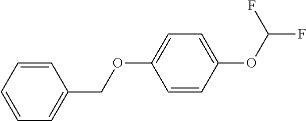 [SMILES: FC(F)Oc1ccc(OCc2ccccc2)cc1]

To a stirring mixture of 4-(benzyloxy)phenol (5.00 g; 4.994 mmol; 1.00 eq.), sodium 2-chloro-2,2-difluoroacetate (5.14 g; 7.491 mmol, 1.50 eq.) in DMF (50 mL) was added. Cs₂CO₃ (13.25 g; 9.988 mmol; 2.00 eq.) and heated to 120° C. for 1 hour under nitrogen atmosphere. The desired product was detected via LCMS. The reaction was quenched with water (30 mL), extracted with EtOAc (3 100 mL). The combined organic layers were washed with brine (3×20 mL), dried over anhydrous Na₂SO₄. After filtration, the filtrate was concentrated under reduced pressure. The residue was purified by flash chromatography on silica gel column using 10-50% of EtOAc in petroleum ether as eluent to provide 1-(benzyloxy)-4-(difluoromethoxy)benzene an off-white solid (3.80 g, 53.1%). LCMS (ESI) m/z 251.1, [M+H]⁺.

### Step 2: 4-(difluoromethoxy)phenol

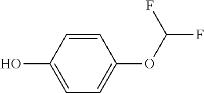 [SMILES: Oc1ccc(OC(F)F)cc1]

To a solution of 1-(benzyloxy)-4-(difluoromethoxy)benzene (3.80 g; 15.19 mmol; 1.00 eq.) in MeOH (100 mL) was added 10% Pd/C (1.14 g; 30% w/w) under nitrogen atmosphere. The mixture was hydrogenated at room temperature for 3 hours under hydrogen atmosphere (2 atm). After filtration, the filtrate was concentrated under reduced pressure to afford 4-(difluoromethoxy)phenol as a yellow solid (2.1 g, crude). LCMS (ESI) m/z 161.0, [M+H]⁺.

### Step 3: 4-(difluoromethoxy)-2-nitrophenol

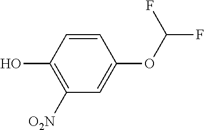 [SMILES: O=[N+]([O-])c1cc(OC(F)F)ccc1O]

A solution of 4-(difluoromethoxy)phenol (620.0 mg, 3.846 mmol; 1.00 eq.) and urea nitrate (472.3 mg; 3.846 mmol; 1.00 eq.) in a mixture solvent of MeCN/water (20:1, 2.1 mL) was stirred at 90° C. for 1 hour. The desired product was detected via LCMS. The reaction was diluted with water (2 mL) and extracted with EtOAc (3×10 mL). The combined organic layers were washed with brine (2×5 mL), dried over anhydrous Na₂SO₄. After filtration, the filtrate was concentrated under reduced pressure. The residue was purified by flash chromatography on silica gel column using 10-40% of EtOAc in petroleum ether as eluent to provide 4-(difluoromethoxy)-2-nitrophenol as a white solid (400.1 mg, 50.7%). LCMS (ESI) m/z 206.0, [M+H]⁺.

### Step 4: 2-amino-4-(difluoromethoxy)phenol

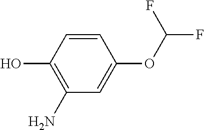 [SMILES: Nc1cc(OC(F)F)ccc1O]

To a solution of 4-(difluoromethoxy)-2-nitrophenol (400.1 mg; 1.950 mmol; 1.00 eq.) in MeOH (10 mL) was added 10% Pd/C (400.1 mg; 100% w/w) under nitrogen atmosphere. The mixture was hydrogenated at room temperature for 3 hours under hydrogen atmosphere (2 atm). After filtration, the filtrate was concentrated under reduced pressure. The residue was purified by flash chromatography on silica gel column using 0-50% of EtOAc in petroleum ether as eluent to provide 2-amino-4-(difluoromethoxy)phenol as a yellow solid (330.1 mg, 97.0%). LCMS (ESI) m/z 176.0, [M+H]⁺.

### Step 5: N-(5-(5-(difluoromethoxy)benzo[d]oxazol-2-yl)-8-(methylamino)-2,7-naphthyridin-3-yl)cyclopropanecarboxamide

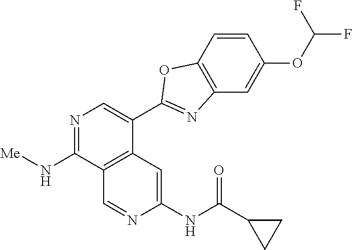 [SMILES: CNc1ncc(-c2nc3cc(OC(F)F)ccc3o2)c2cc(NC(=O)C3CC3)ncc12]

N-(5-(5-(difluoromethoxy)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(difluoromethoxy)phenol as the starting material. LCMS (ESI) m/z 426.1, [M+H]⁺. ¹H NMR (400 MHz, DMSO-d₆) δ 11.06 (s, 1H), 9.67 (s, 9.45 (s, 1H), 8.93 (s, 1H), 8.82-8.49 (m, 1H), 7.78 (d, J=8.8 Hz, 1H), 7.58 (d, J=2.4 Hz, 1H), 7.53-7.11 (m, 2H), 3.10 (d, J=4.4 Hz, 3H), 2.12-2.03 (m, 1H), 0.96-0.90 (m, 2H), 0.90-0.83 (m, 2H).

### Example 226: Synthesis of N-(5-(5-(cyclopropylmethoxy)benzo[d]oxazol-2-yl)-8-(methylamino)-2,7-naphthyridin-3-yl)cyclopropanecarboxamide

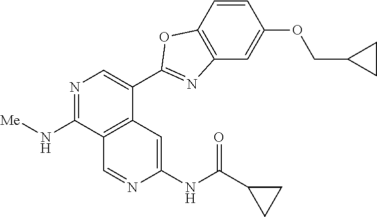 [SMILES: CNc1ncc(-c2nc3cc(OCC4CC4)ccc3o2)c2cc(NC(=O)C3CC3)ncc12]

### Step 1: 1-(benzyloxy)-4-(cyclopropylmethoxy)benzene

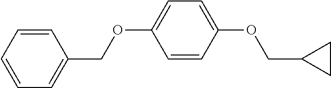 [SMILES: c1ccc(COc2ccc(OCC3CC3)cc2)cc1]

To a mixture of 4-(benzyloxy)phenol (1.00 g; 4.994 mmol; 1.00 eq.) and Cs₂CO₃ (3.26 g; 10.006 mmol; 2.00 eq.) in DMF (10 mL) was added (bromomethyl)cyclopropane (2.00 g; 14.814 mmol; 2.97 eq.) at room temperature. The reaction was stirred at 120° C. for 16 hours under nitrogen atmosphere. The desired product was detected via LCMS. The reaction was diluted with EtOAc (230 mL) and washed with brine (3×10 mL). The organic phase was dried with Na₂SO₄, filtered, and concentrated under reduced pressure. The residue was purified by flash chromatography on silica gel column using 2-10% of EtOAc in petroleum ether as eluent to provide 1-(benzyloxy)-4-(cyclopropylmethoxy)benzene as white solid (2.40 g crude). LCMS (ESI) m/z 255.1, [M+H]⁺.

### Step 2: 4-(cyclopropylmethoxy)phenol

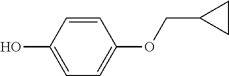 [SMILES: Oc1ccc(OCC2CC2)cc1]

To a solution of 1-(benzyloxy)-4-(cyclopropylmethoxy)benzene 0.20 g; 8.650 mmol; 1.00 eq. MeOH (100 mL) was added 10% Pd/C (660.0 mg; 30% w/w) under nitrogen atmosphere. The mixture was hydrogenated at room temperature for 5 hours under hydrogen atmosphere (2 atm). After filtration, the filtrate was concentrated under reduced pressure to afford 4-(cyclopropylmethoxy)phenol as purple oil (1.40 g, 98.5%), LCMS (ESI) m/z 165.1, [M+H]⁺.

### Step 3: 4-(cyclopropylmethoxy)-2-nitrophenol

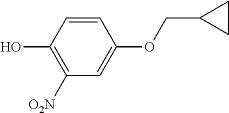 [SMILES: O=[N+]([O-])c1cc(OCC2CC2)ccc1O]

To a solution of 4-(cyclopropylmethoxy)phenol (1.00 g; 6.090 mmol; 1.00 eq.) in MeCN/water (95:1, 10 mL) was added urea nitrate (749.4 mg; 6.090 mmol; 1.00 eq.) under nitrogen atmosphere. The reaction mixture was stirred at 80° C. for 1 hour under microwave radiation. The desired product was detected via LCMS. The reaction was diluted with EtOAc (220 and washed with brine (3×10 mL). The organic phase was dried with Na₂SO₄, filtered, and concentrated under reduced pressure. The residue was purified by flash chromatography on silica gel column using 2-13 of EtOAc in petroleum ether as eluent to provide 4-(cyclopropylmethoxy)-2-nitrophenol as a orange solid (632.0 mg, 49.6%). LCMS (ESI) m/z 210.1, [M+H]⁺.

### Step 4: 2-amino-4-(cyclopropylmethoxy)phenol

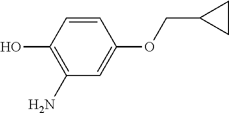 [SMILES: Nc1cc(OCC2CC2)ccc1O]

To a mixture of 4-(cyclopropylmethoxy)-2-nitrophenol (110.0 mg; 0.526 mmol; 1.00 eq.) in MeOH (20 mL) was added 10% Pd/C (110.0 mg; 100% w/w) under nitrogen atmosphere. The mixture was hydrogenated at room temperature for 3 hours under hydrogen atmosphere (2 atm). After filtration, the filtrate was concentrated under reduced pressure to afford 2-amino-4-(cyclopropylmethoxy)phenol as brown solid (82.0 mg, crude). LCMS (ESI) m/z 180.1, [M+H]⁺.

### Step 5: N-(5-(5-(cyclopropylmethoxy)benzo[d]oxazol-2-yl)-8-(methylamino)-2,7-naphthyridin-3-yl)cyclopropanecarboxamide

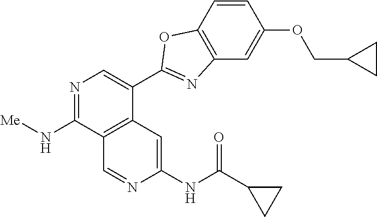 [SMILES: CNc1ncc(-c2nc3cc(OCC4CC4)ccc3o2)c2cc(NC(=O)C3CC3)ncc12]

N-(5-(5-(cyclopropylmethoxy)benzo[d]oxazol-2-yl)-8-(methylamino)-2,7-naphthyridin-3-yl)cyclopropanecarboxamide was synthesized using a similar procedure that was previously described in Example 210 by using N-(5-formyl-8-(methylamino)-2,7-naphthyridin-3-yl)cyclopropanecarboxamide and 2-amino-4-(cyclopropylmethoxy)phenol as the starting material. LCMS (ESI) m/z 430.2, [M+H]⁺. ¹H NMR (400 MHz, DMSO-d₆) δ 11.04 (s, 1H), 9.64 (s, 1H), 9.44 (s, 1H), 8.89 (s, 1H), 8.67-8.61 (m, 1H), 7.61 (d, J=8.8 Hz, 1H), 7.24 (s, 1H), 6.96 (d, 8.8 Hz, 1H), 3.91 (d, J=6.9 Hz, 2H), 3.09 (d, J=4.4 Hz, 3H), 2.14-2.05 (m, 1H), 1.30-1.21 (m, 1H), 0.96-0.81 (m, 4H), 0.64-0.55 (m, 2H), 0.41-0.33 (m, 2H).

### Example 227: Synthesis of N-(5-(5-(2-cyanoethoxy)benzo[d]oxazol-2-yl)-8-(methylamino)-2,7-naphthyridin-3-yl)cyclopropanecarboxamide

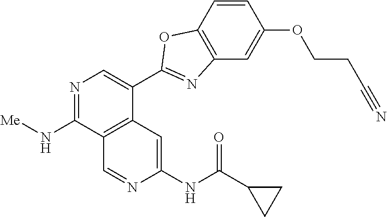 [SMILES: CNc1ncc(-c2nc3cc(OCCC#N)ccc3o2)c2cc(NC(=O)C3CC3)ncc12]

### Step 1: 3-(4-(benzyloxy)phenoxy)propanenitrile

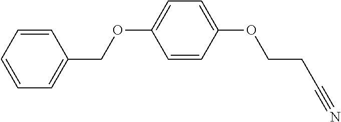 [SMILES: N#CCCOc1ccc(OCc2ccccc2)cc1]

A solution of 4-(benzyloxy)phenol (2.00 g; 9.980 mmol; 1.00 eq.), K₂CO₃ (138.1 mg; 0.999 mmol; 0.10 eq.) and t-BuOH (74.1 mg; 1.000 mmol; 0.10 eq.) in acrylonitrile (20 mL) was stirred at 80° C. for 2 hours under nitrogen atmosphere. The desired product was detected via LCMS. The resulting mixture was concentrated under vacuum. The residue was diluted with CH₂Cl₂ (200 mL), washed with a saturated sodium chloride solution (3×10 mL). The organic layer was dried over anhydrous Na₂SO₄. After filtration, the filtrate was concentrated under reduced pressure to provide 3-(4-(benzyloxy)phenoxy)propanenitrile as a white solid (2.50 g, 98.0%). LCMS (ESI) m/z 252.1, [M−H]⁻.

### Step 2: 3-(4-hydroxyphenoxy)propanenitrile

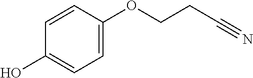 [SMILES: N#CCCOc1ccc(O)cc1]

To a solution of 3-(4-(benzyloxy)phenoxy)propanenitrile (1.00 g; 3.948 mmol; 1.00 eq.) in a mixture solvent of MeOH/CH₂Cl₂ (5:1, 180 mL) was added 10% Pd/C (300.0 mg; 30% w/w) under nitrogen atmosphere. The mixture was hydrogenated at room temperature for 2 hours under hydrogen atmosphere (2 atm). After filtration, the filtrate was concentrated under reduced pressure to provide 3-(4-hydroxyphenoxy)propanenitrile as a brown solid (846.0 mg, 98.1%). LCMS (ESI) m/z 162.1, [M−H]⁻.

### Step 3: 3-(4-hydroxy-3-nitrophenoxy)propanenitrile

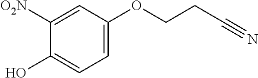 [SMILES: N#CCCOc1ccc(O)c([N+](=O)[O-])c1]

To a stirring mixture of 3-(4-hydroxyphenoxy)propanenitrile (426.0 mg; 2,610 mmol; 1.00 eq.) and urea nitrate (321.3 mg; 2.611 mmol; 1.00 eq.) in MeCN/water (19:1, 10 mL). The resulting mixture was stirred at 80° C. for 2 hours under nitrogen atmosphere. The desired product was detected via LCMS. The resulting mixture was concentrated under reduced pressure. The residue was purified by flash chromatography on silica gel column using 10-30% of EtOAc in petroleum ether as eluent to provide 3-(4-hydroxy-3-nitrophenoxy)propanenitrile as a yellow solid (342.0 mg, 63.0%). LEWIS (ESI) m/z 207.0, [M−H]⁻.

### Step 4: 3-(3-amino-4-hydroxyphenoxy)propanenitrile

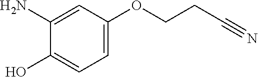 [SMILES: N#CCCOc1ccc(O)c(N)c1]

To a solution of 3-(4-hydroxy-3-nitrophenoxy)propanenitrile (249.0 mg; 1,196 mmol; 1.00 eq.) in MeOH/CH₂Cl₂ (3.5:1, 45 mL) was added 10% Pd/C (74.7 mg; 30% w/w) under nitrogen atmosphere. The mixture was hydrogenated at room temperature for 2 hours under hydrogen atmosphere (2 atm). After filtration, the filtrate was concentrated under reduced pressure to provide 3-(3-amino-4-hydroxyphenoxy)propanenitrile as a brown solid (232.9 mg, 99.1%). LCMS (ESI) in/z 179.1, [M+H]⁺.

### Step 5: N-(5-(5-(2-cyanoethoxy)benzo(dioxazol-2-yl)-8-(methylamino)-2,7-naphthyridin-3-yl)cyclopropanecarboxamide

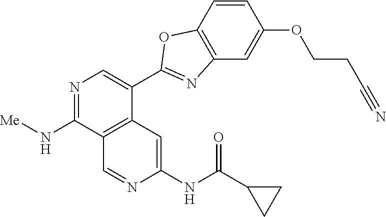 [SMILES: CNc1ncc(-c2nc3cc(OCCC#N)ccc3o2)c2cc(NC(=O)C3CC3)ncc12]

N-(5-(5-(2-cyanoethoxy)benzo[d]oxazol-2-yl)-8-(methyl amino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 3-(3-amino-4-hydroxyphenoxy)propanenitrile as the starting material, LCMS (ESI) nor/z: 429.2, [M+H]⁺. ¹H NMR, (400 MHz, DMSO-d₆) δ 11.03 (s, 1H), 9.65 (s, 1H), 9.44 (s, 1H), 8.90 (s, 1H), 8.67-8.63 (m, 1H), 7.65 (d, J=8.8 Hz, 1H), 7.35 (d, J=2.4 Hz, 1H), 7.01 (dd, J=8.8, 2.4 Hz, 1H), 4.30 (t, J=6.0 Hz, 2H), 3.09 (d, J=4.4 Hz, 3H), 3.04 (t, J=6.0 Hz, 2H), 2.14-2.07 (m, 1H), 0.95-0.82 (m, 4H).

### Example 228: Synthesis of N-(8-(methylamino)-5-(5-(oxetan-3-yloxy)benzo[d]oxazol-2-yl)-2,7-naphthyridin-3-yl)cyclopropanecarboxamide

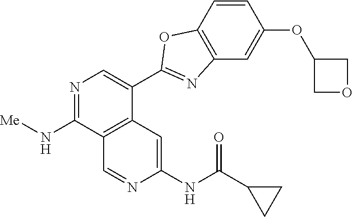 [SMILES: CNc1ncc(-c2nc3cc(OC4COC4)ccc3o2)c2cc(NC(=O)C3CC3)ncc12]

### Step 1: 3-(4-(benzyloxy)phenoxy)oxetane

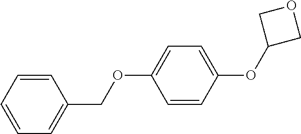 [SMILES: c1ccc(COc2ccc(OC3COC3)cc2)cc1]

To a stirring mixture of 4-(benzyloxy)phenol (2.00 g; 9,990 mmol; 1.00 eq.), 3-bromooxetane (4.10 g; 29.930 mmol; 3.00 eq.) and Cs₂CO₃ (9.76 g; 29.96 mmol; 2.00 eq.) in DMF (24 mL) was stirred at 100° C. for 14 hours under nitrogen atmosphere. The desired product was detected via, LCMS. The resulting mixture was diluted with EtOAc (100 mL) and washed with brine (3×10 mL). The organic layer was dried over anhydrous Na₂SO₄. After filtration, the filtrate was concentrated under reduced pressure. The residue was purified by flash chromatography on silica gel column using 15-40% of EtOAc in petroleum ether as eluent to provide 3-(4-(benzyloxy)phenoxy)oxetane as a white solid (1.43 g, 55.9%). LCMS (ESI) m/z 257.1, [M+H]⁺.

### Step 2: 4-(oxetan-3-yloxy)phenol

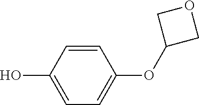 [SMILES: Oc1ccc(OC2COC2)cc1]

To a stirring mixture of 3-(4-(benzyloxy)phenoxy)oxetane (1.57 g; 6.125 mmol; 1.00 eq.) in MeOH (50 mL) was added 10% Pd/C (530.1 mg; 30% w/w) under nitrogen atmosphere. The mixture was hydrogenated at room temperature for 3 hours under hydrogen atmosphere (2 atm). After filtration, the filtrate was concentrated under reduced pressure to afford 4-(oxetan-3-yloxy)phenol as a yellow solid (860.0 mg, crude). LCMS (ESI) m/z 167.1, [M+H]⁺.

### Step 3: 2-nitro-4-(oxetan-3-yloxy)phenol

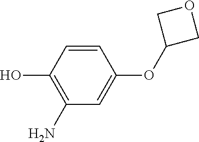 [SMILES: Nc1cc(OC2COC2)ccc1O]

A solution of 4-(oxetan-3-yloxy)phenol (860.0 mg; 5.175 mmol; 1.00 eq.) and urea nitrate (636.9 mg; 5.175 mmol; 1.00 eq.) in MeCN/water (20:1, 10 mL) was stirred at 80° C. for 2 hours under nitrogen atmosphere. The resulting mixture was diluted with water (30 mL), extracted with EtOAc (3×40 mL). The combined organic layers were washed with brine (3×10 mL), dried over anhydrous Na₂SO₄. After filtration, the filtrate was concentrated under reduced pressure. The residue was purified by flash chromatography on silica gel column using 0-10% of MeOH in CH₂Cl₂ as eluent to provide 2-nitro-4-(oxetan-3-yloxy)phenol as a grey solid (510.1 mg, 46.7%). LCMS (ESI) m/z 212.0, [M+H]⁺.

### Step 4: 2-amino-4-(oxetan-3-yloxy)phenol

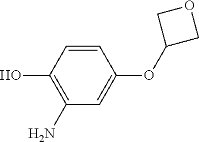 [SMILES: Nc1cc(OC2COC2)ccc1O]

A solution of 2-nitro-4-(oxetan-3-yloxy)phenol (190.1 mg; 0.794 mmol; 1.00 eq.) in MeOH (5 mL) was added 10% Pd/C (190.1 mg; 100% w/w) under nitrogen atmosphere. The mixture was hydrogenated at room temperature for 3 hours under hydrogen atmosphere (2 atm). After filtration, the filtrate was concentrated under reduced pressure. The residue was purified by flash chromatography on silica gel column using 0-5% of MeOH in CH₂Cl₂ as eluent to provide 2-amino-4-(oxetan-3-yloxy)phenol as a black solid (90.0 mg, 95.4%).

### Step 5: N-(8-(methylamino)-5-(5-(oxetan-3-yloxy)benzo[d]oxazol-2-yl)-2,7-naphthyridin-3-yl)cyclopropanecarboxamide

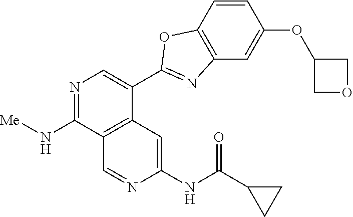 [SMILES: CNc1ncc(-c2nc3cc(OC4COC4)ccc3o2)c2cc(NC(=O)C3CC3)ncc12]

N-(8-(methylamino)-5-(5-(oxetan-3-yloxy)benzo[d]0 oxazol-2-yl)-2, 7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(oxetan-3-yloxy)phenol as the starting material. LCMS (ESI) m/z 432.2, [M+H]⁺. ¹H NMR (400 MHz, DMSO-d₆) δ 11.03 (s, 1H) 9.56 (s, 1H), 9.43 (s, 1H), 8.88 (s, 1H), 8.75-8.45 (m, 1H), 7.64 (d, J=8.8 Hz, 1H), 7.08 (d, J=2.4 Hz, 1H), 6.99-6.71 (m, 1H), 5.53-5.25 (m, 1H), 5.10-4.88 (m, 2H), 4.79-4.31 (m, 2H), 3.08 (d, J=4.4 Hz, 3H), 2.12-2.06 (m, 1H), 0.99-0.79 (m, 4H).

### Example 229: Synthesis of N-(8-(methylamino)-5-(5-((tetrahydrofuran-3-yl)oxy)benzo[d]oxazol-2-yl)-2,7-naphthyridin-3-yl) cyclopropanecarboxamide

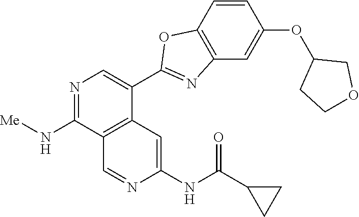 [SMILES: CNc1ncc(-c2nc3cc(OC4CCOC4)ccc3o2)c2cc(NC(=O)C3CC3)ncc12]

### Step 1: 3-(4-(benzyloxy)phenoxy)tetrahydrofuran

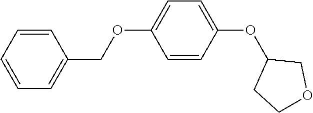 [SMILES: c1ccc(COc2ccc(OC3CCOC3)cc2)cc1]

A solution of 4-(benzyloxy)phenol (1.70 g; 8.490 mmol; 1.00 eq.), Cs₂CO₃ (5.53 g; 16.980 mmol; 2.00 eq.) and 3-bromotetrahydrofuran (2.56 g; 16.980 mmol; 2.00 eq.) in DMF (15 mL) was stirred at 120° C. overnight under nitrogen atmosphere. The resulting mixture was diluted with EtOAc (150 mL) and washed with brine (3×10 mL). The organic layer was dried over anhydrous Na₂SO₄. After filtration, the filtrate was concentrated under reduced pressure. The residue was purified by flash chromatography on silica gel column using 0-30% of EtOAc in petroleum ether as eluent to provide 3-(4-(benzyloxy)phenoxy)tetrahydrofuran as an off-white solid (1.21 g, 52.7%). LCMS (ESI) m/z 271.0, [M+H]⁺.

### Step 2: 4-((tetrahydrofuran-3-yl)oxy)phenol

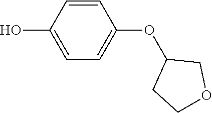 [SMILES: Oc1ccc(OC2CCOC2)cc1]

To a solution of 3-(4-(benzyloxy)phenoxy)tetrahydrofuran (1.21 g; 4.470 mmol; 1.00 eq.) in MeOH (100 mL) was added 10% Pd/C (363.1 mg; 30% w/w) under nitrogen atmosphere. The mixture was hydrogenated for 3 hours at room temperature under hydrogen atmosphere (2 atm). After filtration, the filtrate was concentrated under reduced pressure to afford 4-((tetrahydrofuran-3-yl)oxy)phenol as a yellow solid (850 mg, crude). LCMS (ESI) m/z 181.0, [M+H]⁺.

### Step 3: 2-nitro-4-((tetrahydrofuran-3-yl)oxy)phenol

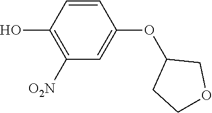 [SMILES: O=[N+]([O-])c1cc(OC2CCOC2)ccc1O]

A mixture of urea nitrate (273.1 mg; 2.220 mmol; 1.00 eq.) and 4-((tetrahydrofuran-3-yl)oxy)phenol (400.1 mg; 2.220 mmol; 1.00 eq.) in a mixture solvent of MeCN/water (20:1, 4.2 mL) was stirred at 80° C. for 1 hour under nitrogen atmosphere. The resulting mixture was diluted with CH₂Cl₂ (50 mL), washed with brine (2×10 mL). The organic phase was dried over anhydrous Na₂SO₄. After filtration, the filtrate was concentrated under reduced pressure. The residue was purified by flash chromatography on silica gel column using 10-40% of EtOAc in petroleum ether as eluent to provide 2-nitro-4-((tetrahydrofuran-3-yl)oxy)phenol as a yellow solid (340.2 mg, 68.0%). LCMS (ESI) m/z 226.1, [M+H]⁺.

### Step 4: 2-amino-4-((tetrahydrofuran-3-yl)oxy)phenol

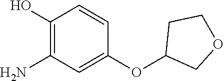 [SMILES: Nc1cc(OC2CCOC2)ccc1O]

A solution of 2-nitro-4-((tetrahydrofuran-3-yl)oxy)phenol (340.2 mg; 1,510 mmol; 1.00 eq.) in MeOH (20 mL) was added 10% Pd/C (340.2 mg; 100% w/w) under nitrogen atmosphere. The mixture was hydrogenated at room temperature for 3 hours under hydrogen atmosphere (2 atm). After filtration, the filtrate was concentrated under reduced pressure to afford 2-amino-4-((tetrahydrofuran-3-yl)oxy)phenol as a brown solid (240 mg, crude). LCMS (ESI) m/z 196.1, [M+H]⁺.

### Step 5: N-(8-(methylamino)-5-(5-((tetrahydrofuran-3-yl)oxy)benzo[d]oxazol-2-yl)-2,7-naphthyridin-3-yl)cyclopropanecarboxamide

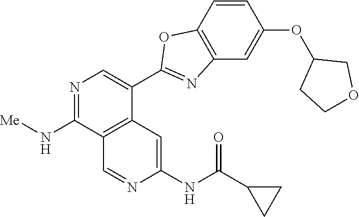 [SMILES: CNc1ncc(-c2nc3cc(OC4CCOC4)ccc3o2)c2cc(NC(=O)C3CC3)ncc12]

N-(8-(methylamino)-5-(5-((tetrahydrofuran-3-yl)oxy)benzo[d]oxazol-2-yl)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-((tetrahydrofuran-3-yl)oxy)phenol as the starting material. LCMS (ESI) m/z 446.2, [M+H]⁺. NMR (400 MHz, DMSO-d₆) 11.03 (s, 1H), 9.60 (s, 1H), 9.44 (s, 1H), 8.89 (s, 1H), 8.75-8.44 (m, 1H), 7.62 (d, J=8.8 Hz, 1H), 7.28 (d, J=2.4 Hz, 1H), 7.07-6.72 (m, 1H), 5.27-4.99 (m, 1H, 3.98-3.72 (m, 4H), 3.09 (d, J=4.4 Hz, 3H), 2.32-2.19 (m, 1H), 2.16-1.98 (m, 1H), 0.98-0.79 (m, 4H).

### Example 230: Synthesis of N-(5-(5-((1-acetylazetidin-3-yl)oxy)benzo[d]oxazol-2-yl)-8-(methylamino)-2,7-naphthyridin-3-yl)cyclopropanecarboxamide

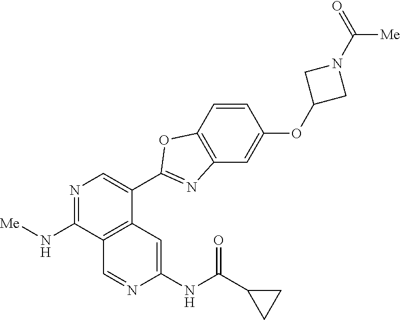 [SMILES: CNc1ncc(-c2nc3cc(OC4CN(C(C)=O)C4)ccc3o2)c2cc(NC(=O)C3CC3)ncc12]

### Step 1: tert-butyl 3-(1-(benzyloxy)phenoxy)azetidine-1-carboxylate

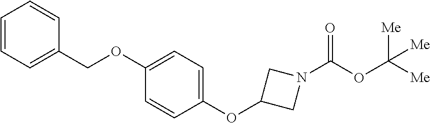 [SMILES: CC(C)(C)OC(=O)N1CC(Oc2ccc(OCc3ccccc3)cc2)C1]

A mixture of 4-(benzyloxy)phenol (2.00 g; 9.999 mmol; 1.00 eq.), Cs₂CO₃ (6.51 g; 19.998 mmol; 2.00 eq.) and tert-butyl 3-bromoazetidine-1-carboxylate (3.54 g; 14.997 mmol; 1.50 eq.) in DMF (20 mL) was stirred at 120° C. for 1 hour under nitrogen atmosphere. After the reaction was completed, the mixture was diluted with EtOAc (100 mL), washed with water (3×20 mL). The organic phase was concentrated under reduced pressure. The residue was purified by flash chromatography on silica gel column using 0-30% of EtOAc in petroleum ether as eluent to provide tert-butyl 3-(4-(benzyloxy)phenoxy)azetidine-1-carboxylate as a white solid (3.40 g, 95.6%).

### Step 2: 3-(4-(benzyloxy)phenoxy)azetidine hydrochloride

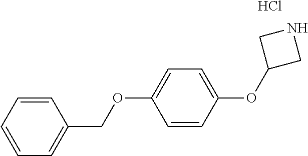 [SMILES: Cl ; c1ccc(COc2ccc(OC3CNC3)cc2)cc1]

To a stirred mixture of tert-butyl 3-(4-(benzyloxy)phenoxy)azetidine-1-carboxylate (2.40 g; 6.752 mmol; 1.00 eq.) in MeOH (10 mL) was added HCl (gas) (4 M in 1,4-dioxane, 20 mL) at room temperature. The resulting solution was stirred at room temperature for 2 hours. The mixture was concentrated under reduced pressure to afford 3-(4-(benzyloxy)phenoxy)azetidine hydrochloride as a white solid (1.90 g, 96.4%). LCMS (ESI) m/z 256.1, [M+H]⁺.

### Step 3: 1-(3-(4-(benzyloxy)phenoxy)azetidin-1-yl)ethan-1-one

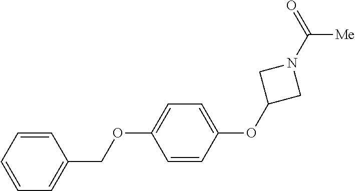 [SMILES: CC(=O)N1CC(Oc2ccc(OCc3ccccc3)cc2)C1]

To a solution of 3-(4-(benzyloxy)phenoxy)azetidine hydrochloride (2.00 g; 6.854 mmol; 1.00 eq.) and DIPEA (5.32 g; 41.193 mmol; 6.01 eq.) in CH₂Cl₂ (20.0 mL) was added acetic anhydride (1.40 g; 13.71 mmol; 2.00 eq.) under nitrogen atmosphere. The resulting mixture was stirred at room temperature for 3 hours. The desired product was detected via LCMS. The resulting mixture was diluted with CH₂Cl₂ (100 mL) and washed with brine. The organic phase was concentrated under reduced pressure. The residue was purified by flash chromatography on silica gel column using 0-7% of MeOH in CH₂Cl₂ as eluent to provide 1-(3-(4-(benzyloxy)phenoxy)azetidin-1-yl)ethan-1-one as an off-white solid (1.23 g, 59.1%). LCMS (ESI) m/z 298.1, [M+H]⁺.

### Step 4: 1-(3-(4-hydroxyphenoxy)azetidin-1-yl)ethan-1-one

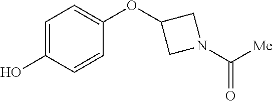 [SMILES: CC(=O)N1CC(Oc2ccc(O)cc2)C1]

To a stirring mixture of 1-(3-(4-(benzyloxy)phenoxy)azetidin-1-yl)ethan-1-one (1.23 g; 4.14 mmol; 1.00 eq.) in MeOH (80 mL) was added 10% Pd/C (1.23 g; 100% w/w) under nitrogen atmosphere. The mixture was hydrogenated at 40° C. for 2 hours under hydrogen atmosphere (2 atm). After filtering through a celite pad, the filtrate was concentrated under reduced pressure to afford 1-(3-(4-hydroxyphenoxy)azetidin-1-yl)ethan-1-one as a grey solid (682.0 mg, 74.7%). LCMS (ESI) m/z 208.1, [M+H]⁺.

### Step 5: 1-(3-(4-hydroxy-3-nitrophenoxy)azetidin-1-yl)ethan-1-one

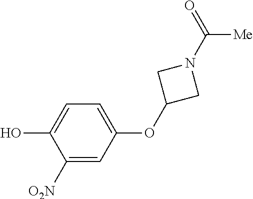 [SMILES: CC(=O)N1CC(Oc2ccc(O)c([N+](=O)[O-])c2)C1]

A solution of 1-(3-(4-hydroxyphenoxy)azetidin-1-yl)ethan-1-one (682.0 mg; 3.291 mmol, 1.00 eq.) and urea nitrate (400.9 mg; 3.26 mmol, 0.99 eq.) in a mixture of acetonitrile/water (20:1, 8 mL) was stirred at 80° C. for 1 hour. The desired product was detected via LCMS. The resulting mixture was diluted with EtOAc (100 mL) and washed with brine. The organic phase was concentrated under reduced pressure. The residue was purified by flash chromatography on silica gel column using 0-5% of MeOH in CH₂Cl₂ as eluent to provide 1-(3-(4-hydroxy-3-nitrophenoxy)azetidin-1-yl)ethan-1-one as a yellow solid (565.0 mg, 63.3%). LCMS (ESI) m/z 251.1, [M+H]⁺.

### Step 6: 1-(3-(3-amino-4-hydroxyphenoxy)azetidin-1-yl)ethan-1-one

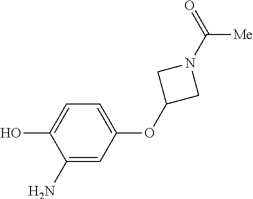 [SMILES: CC(=O)N1CC(Oc2ccc(O)c(N)c2)C1]

To a solution of 1-(3-(4-hydroxy-3-nitrophenoxy)azetidin-1-yl)ethan-1-one (515.0 mg; 2.042 mmol, 1.00 eq.) in MeOH (30 mL) was added 10% Pd/C (515.0 mg; 100% w/w) under nitrogen atmosphere. The mixture was hydrogenated at 40° C. for 2 hours under hydrogen atmosphere (2 atm). After filtration, the filtrate was concentrated under reduced pressure to afford 1-(3-(3-amino-4-hydroxyphenoxy)azetidin-1-yl)ethan-1-one as a brown solid (397.0 mg, 69.9%). LCMS (ESI) m/z 223.1, [M+H]⁺.

### Step 7: N-(5-(54(1-acetylazetidin-3-yl)oxy)benzo[d]oxazol-2-yl)-8-(methylamino)-2,7-naphthyridin-3-yl)cyclopropanecarboxamide

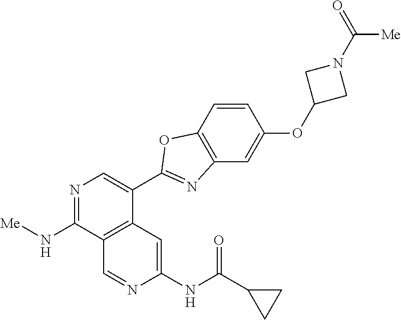 [SMILES: CNc1ncc(-c2nc3cc(OC4CN(C(C)=O)C4)ccc3o2)c2cc(NC(=O)C3CC3)ncc12]

N-(5-(5-((1-acetylazetidin-3-yl)oxy)benzo[d]oxazol-2-yl)-84 methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 1-(3-(3-amino-4-hydroxyphenoxy)azetidin-1-yl)ethan-1-one as the starting material, LCMS (ESI) m/z 473.2, [M+H]⁺. ¹H NMR (400 MHz, DMSO-d₆) δ 11.03 (s, 1H), 9.57 (s, 1H), 9.44 (s, 1H), 8.89 (5.1H), 8.69-8.60 (m, 1H), 7.66 (d, J=8.8 Hz, 1H), 7.17 (d, J=2.4 Hz, 1H), 6.94 (dd, J=8.8, 2.4 Hz, 1H), 5.20-5.11 (m, 1H), 4.67-4.54 (m, 1H), 4.37-4.28 (m, 1H), 4.17-4.09 (m, 1H), 3.88-3.76 (m, 1H), 3.09 (d, J=4.4 Hz, 3H), 2.16-2.05 (m, 1H), 1.82 (s, 3H), 0.96-0.80 (m, 4H).

### Example 231: Synthesis of N-(5-(5-(2-methoxyethoxy)benzo[d]oxazol-2-yl)-8-(methylamino 2,7-naphthyridin-3-yl)cyclopropanecarboxamide

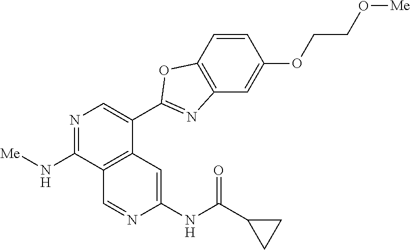 [SMILES: CNc1ncc(-c2nc3cc(OCCOC)ccc3o2)c2cc(NC(=O)C3CC3)ncc12]

### Step 1: 1-(benzyloxy)-4-(2-methoxyethoxy)benzene

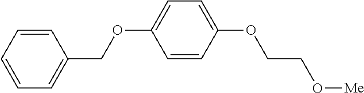 [SMILES: COCCOc1ccc(OCc2ccccc2)cc1]

To a stirring mixture of 4-(benzyloxy)phenol (5.00 g; 24.970 mmol; 1.00 eq.), 1-bromo-2-methoxyethane (10.35 g; 74,465 mmol; 2.98 eq.) in DMF (60 mL) was added Cs₂CO₃ (16.30 g; 50.028 mmol; 2.00 eq.). The resulting mixture was stirred at 120° C. overnight. The desired product was detected via LCMS. The mixture was allowed to cool down to room temperature. The resulting mixture was diluted with water (200 mL) and extracted with EtOAc (4×100 mL). The combined organic layers were washed with brine (4×100 mL), dried over anhydrous Na₂SO₄. After filtration, the filtrate was concentrated under reduced pressure. The residue was purified by flash chromatography on silica gel column using 50-70% of EtOAc in petroleum ether as eluent to provide 1-(benzyloxy)-4-(2-methoxyethoxy)-benzene as a white solid (5.85 g, 90.6%). LCMS (ESI) m/z 259.1, [M+H]⁺.

### Step 2: 4-(2-methoxyethoxy)phenol

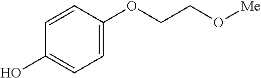 [SMILES: COCCOc1ccc(O)cc1]

To a stirring mixture of 1-(benzyloxy)-4-(2-methoxyethoxy)benzene (2.50 g; 9.678 mmol; 1.00 eq.) in MeOH (140 mL) was added 10% Pd/C (0.75 g, 30% w/w) under nitrogen atmosphere. The mixture was hydrogenated at room temperature for 3 hours under hydrogen atmosphere (2 atm). The resulting mixture was filtered, the filter cake was washed with MeOH (4×20 mL). The filtrate was collected and concentrated under reduced pressure to afford 4-(2-methoxyethoxy)phenol as a light brown solid (1.30 g, 79.8%). LCMS (ESI) m/z 169.1, [M+H]⁺.

### Step 3: 4-(2-methoxyethoxy)-2-nitrophenol

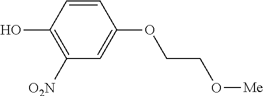 [SMILES: COCCOc1ccc(O)c([N+](=O)[O-])c1]

To a stirring mixture of urea nitrate (943.9 mg; 7.670 mmol; 1.00 eq.) in MeCN/water (95:5, 10 mL) was added 4-(2-methoxyethoxy)phenol (1.29 g; 7.670 mmol; 1.00 eq.). The resulting mixture was stirred at 80° C. for 1 hour under nitrogen atmosphere. The reaction mixture was reacted under a microwave irradiation at 80° C. for 1 hour. The mixture was allowed to cool down to room temperature. The resulting mixture was diluted with water (50 mL) and extracted with EtOAc (4×30 mL). The combined organic layers were washed with brine (3×30 mL), dried over anhydrous Na₂SO₄. After filtration, the filtrate was concentrated under reduced pressure. The solvent was concentrated under vacuum. The residue was purified by flash chromatography on silica gel column using 10-20% of EtOAc in petroleum ether as eluent to provide 4-(2-methoxyethoxy)-2-nitrophenol as a yellow solid (650.2 mg, 39.7%). LCMS (ESI) m/z 214.1, [M+H]⁺.

### Step 4: 2-amino-4-(2-methoxyethoxy)phenol

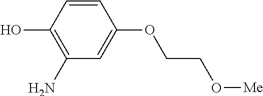 [SMILES: COCCOc1ccc(O)c(N)c1]

To a stirring mixture of 4-(2-methoxyethoxy)-2-nitrophenol (650.0 mg; 3.049 mmol; 1.00 eq.) in MeOH (22 was added 10% Pd/C (650 mg, 100% w/w) under nitrogen atmosphere. The mixture was hydrogenated at room temperature for 3 hours under hydrogen atmosphere (2 atm). The desired product was detected via LCMS. The resulting mixture was filtered, the filter cake was washed with MeOH (5×10 mL). The filtrate was concentrated under reduced pressure. The residue was purified by flash chromatography on silica gel column using 40-50% of EtOAc in petroleum ether as eluent to provide 2-amino-4-(2-methoxyethoxy)phenol as a brown oil (390.0 mg, 69.8%). LCMS (ESI) m/z 184.1, [M+H]⁺. Step 5: N-(5-(5-(2-methoxyethoxy)benzo[d]oxazol-2-yl)-8-(methylamino)-2,7-naphthyridin-3-yl)cyclopropanecarboxamide

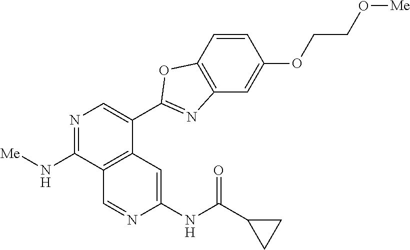 [SMILES: CNc1ncc(-c2nc3cc(OCCOC)ccc3o2)c2cc(NC(=O)C3CC3)ncc12]

N-(5-(5-(2-methoxyethoxy)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(2-methoxyethoxy)phenol as the starting material. LCMS (ESI) m/z 434.2, [M+H]⁺. ¹H NMR (400 MHz, DMSO-d₆) δ 11.05 (s, 1H), 9.64 (s, 1H), 9.44 (s, 1H), 8.90 (s, 1H), 8.69-8.63 (m, 1H), 7.62 (d, J=8.8 Hz, 1H), 7.30 (d, J=2.4 Hz, 1H), 6.97 (dd, J=8.8, 2.4 Hz, 1H), 4.22-4.18 (m, 2H), 3.73-3.69 (m, 2H), 3.34 (s, 3H), 3.09 (d, J=4.4 Hz, 3H), 2.14-2.06 (m, 1H), 0.93-0.84 (m, 4H).

### Example 232: Synthesis of N-(5-(5-((1s,3s)-3-cyanocyclobutoxy)benzo[d]oxazol-2-yl)-8-(methylamino)-2,7-naphthyridin-3-yl)cyclopropanecarboxamide

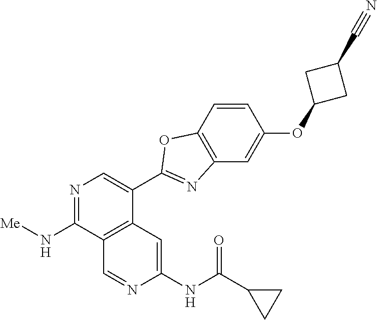 [SMILES: CNc1ncc(-c2nc3cc(O[C@H]4C[C@@H](C#N)C4)ccc3o2)c2cc(NC(=O)C3CC3)ncc12]

### Step 1: 3-cyanocyclobutyl ethanesulfonate

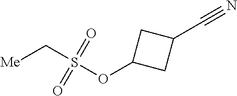 [SMILES: CCS(=O)(=O)OC1CC(C#N)C1]

To a stilling mixture of 3-hydroxycyclobutane-1-carbonitrile (500.0 mg; 5.148 mmol; 1.00 eq.) and Et₃N (1.56 g; 15.42 mmol; 2.99 eq.) in CH₂Cl₂ (15 mL) was added ethane sulfonyl chloride (993.0 mg; 7.72 mmol; 1.50 eq.) at 0° C. The reaction mixture was stirred at room temperature for 4 hours under nitrogen atmosphere. The desired product was detected via TLC. The resulting mixture was diluted with water (100 mL), extracted with EtOAc (3×100 mL). The combined organic layers were concentrated under vacuum to afford 3-cyanocyclobutyl ethanesulfonate as aN off-white oil (770.0 mg, crude). The crude product was used in the next step directly without further purification.

### Step 2: (1S,3S)-3-(4-(benzyloxy)phenoxy)cyclobutane-1-carbonitrile and (1R,3R)-3-(4-(benzyloxy)phenoxy)cyclobutane-1-carbonitrile

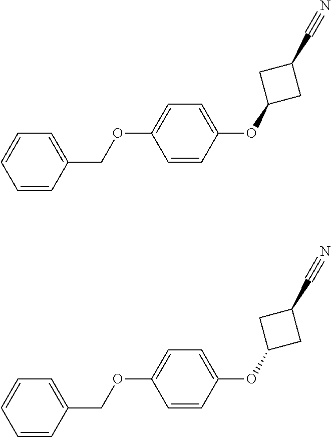 [SMILES: N#C[C@H]1C[C@@H](Oc2ccc(OCc3ccccc3)cc2)C1 ; N#C[C@H]1C[C@H](Oc2ccc(OCc3ccccc3)cc2)C1]

To a stirring mixture of 3-cyanocyclobutyl ethanesulfonate (770.0 mg; 4.069 mmol; 1.00 eq.), 4-(benzyloxy)phenol (978.0 mg; 4.884 mmol; 1.20 eq.) in IMF (10 was added Cs₂CO₃ (3.98 g; 12.22 mmol; 3.00 eq.). The resulting mixture was stirred at 85 DC overnight. The desired product was detected via LCMS. The resulting mixture was diluted with water (100 mL), extracted with EtOAc (3×100 mL). The combined organic layers were concentrated under vacuum. The residue was purified by flash chromatography on pre-packed C18 column using 50-70% of MeCN in water (10 mmol/L NH₄HCO₃) as eluent to provide the crude product. The crude product was purified by flash chromatography on silica gel column using 10-30% of EtOAc in petroleum ether as eluent to provide (1s,3s)-3-(4-(benzyloxy)phenoxy)cyclobutane-1-carbonitrile (the faster peak) as an off-white solid (539.0 mg, 47.2%) and (1r,3r)-3-(4-(benzyloxy)phenoxy)cyclobutane-1-carbonitrile (the slower peak) as an off-white solid (3060 mg, 26.9%). LCMS m/z 278.1, [M+H]⁺.

### Step 3: (1S,3S)-3-(4-hydroxyphenoxy)cyclobutane-1-carbonitrile

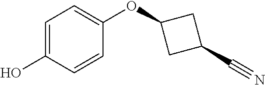 [SMILES: N#C[C@H]1C[C@@H](Oc2ccc(O)cc2)C1]

To a stirring mixture of (1S,3S)-3-(4-(benzyloxy)phenoxy)cyclobutane-1-carbonitrile (530.0 mg; 1.897 mmol, 1.00 eq.) in MeOH (1.0 mL) was added 10% Pd/C (53.0 mg; 10% w/w) under nitrogen atmosphere. The mixture was hydrogenated at room temperature for 1 hour under hydrogen atmosphere (2 atm). The desired product was detected via LCMS. The resulting mixture was filtered, the filter cake was washed with MeOH (3×10 mL) and the filtrate was concentrated under reduced pressure. The residue was purified by flash chromatography on silica gel column using 10-90% of EtOAc in petroleum ether as eluent to provide (1s,3s)-3-(4-hydroxyphenoxy)cyclobutane-1-carbonitrile as an off-white solid (310.0 mg; 86.3%). LCMS (ESI) m/z 188.1, [M+H]⁺.

### Step 4: (1s,3s)-3-(4-hydroxy-3-nitrophenoxy)cyclobutane-1-carbonitrile

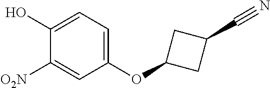 [SMILES: N#C[C@H]1C[C@@H](Oc2ccc(O)c([N+](=O)[O-])c2)C1]

To a stirring mixture of (1S,3S)-3-(4-hydroxyphenoxy)cyclobutane-1-carbonitrile (310.0 mg; 1.64 mmol; 1.00 eq.) in MeCN/water (19:1, 10 mL) was added urea nitrate (202.0 mg; 1.64 mmol; 1.00 eq.). The resulting mixture was stirred at 80° C. overnight. The desired product was detected via. LCMS. The resulting mixture was concentrated under reduced pressure. The residue was purified by flash chromatography on silica gel column using 20-80% of EtOAc in petroleum ether as eluent to provide (1s,3s)-3-(4-hydroxy-3-nitrophenoxy)cyclobutane-1-carbonitrile as a yellow solid (213.0 mg, 55.5%). LCMS (ESI) m/z 233.1, [M−H]⁻.

### Step 5: (1S,1S)-3-(3-amino-4-hydroxyphenoxy)cyclobutane-1-carbonitrile

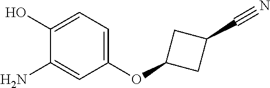 [SMILES: N#C[C@H]1C[C@@H](Oc2ccc(O)c(N)c2)C1]

To a stirring mixture of (1S,3S)-3-(4-hydroxy-3-nitrophenoxy)cyclobutane-1-carbonitrile (213.0 mg; 0.909 mmol; 1.00 eq.) in MeOH (10 mL) was added 10% Pd/C (22.6 Trig; 10% w/w) under nitrogen atmosphere. The mixture was hydrogenated at room temperature for 1 hour under hydrogen atmosphere (2 atm). The desired product was detected via LCMS. The resulting mixture was filtered, the filter cake was washed with MeOH (3×10 mL). The filtrate was concentrated under reduced pressure. The residue was purified by flash chromatography on silica gel column using 50-90% of EtOAc in petroleum ether as eluent to provide (1s,3s)-3-(3-amino-4-hydroxyphenoxy)cyclobutane-1-carbonitrile as a yellow solid (168.0 mg, 90.4%). LCMS (ESI) m/z 205.1, [M+H]⁺.

### Step 6: N-(5-(5-((1s,3s)-3-cyanocyclobutoxy)benzo[d]oxazol-2-yl)-8-(methylamino)-2,7-naphthyridin-3-yl)cyclopropanecarboxamide

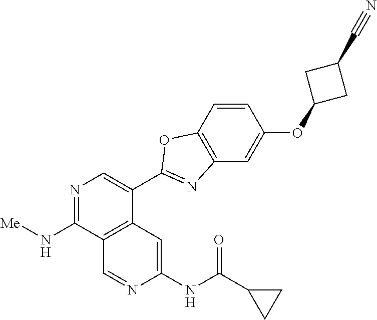 [SMILES: CNc1ncc(-c2nc3cc(O[C@H]4C[C@@H](C#N)C4)ccc3o2)c2cc(NC(=O)C3CC3)ncc12]

N-(5-(5-((1s,3s)-3-cyanocyclobutoxy)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and (1s,3s)-3-(3-amino-4-hydroxyphenoxy)cyclobutane-1-carbonitrile as the starting material. LCMS (ESI) m/z 455.2, [M+H]⁺. ¹H NMR (300 MHz, DMSO-d₆) δ 11.04 (s, 1H), 9.57 (s, 1H), 9.43 (s, 1H), 8.88 (s, 1H), 8.68-8.62 (m, 1H), 7.62 (d, J=8.8 Hz, 1H), 7.17 (d, J=2.4 Hz, 1H), 6.91 (dd, J=8.8, 2.4 Hz, 1H), 5.14-5.04 (m, 1H), 3.53-3.42 (m, 1H), 3.09 (d, 4.4 Hz, 3H), 2.90-2.80 (m, 2H), 2.60-2.52 (n 2H), 2.14-2.07 (m, 1H), 0.93-0.83 (m, 4H).

### Example 233: Synthesis of N-(5-(5-((1r,3r)-3-cyanocyclobutoxy)benzo[d]oxazol-2-yl)-8-(methylamino)-2,7-naphthyridin-3-yl)cyclopropanecarboxamide

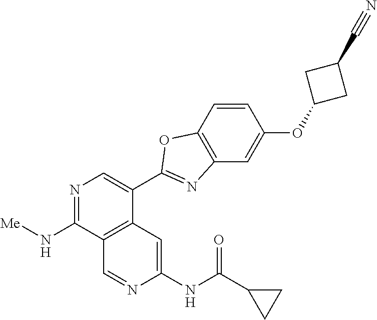 [SMILES: CNc1ncc(-c2nc3cc(O[C@H]4C[C@H](C#N)C4)ccc3o2)c2cc(NC(=O)C3CC3)ncc12]

### Step 1: (1R,3R)-3-(4-hydroxyphenoxy)cyclobutane-1-carbonitrile

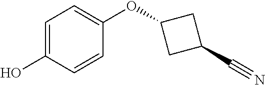 [SMILES: N#C[C@H]1C[C@H](Oc2ccc(O)cc2)C1]

To a stirring mixture of (1r,3r)-3-(4-(benzyloxy)phenoxy)cyclobutane-1-carbonitrile (Example 232, step 2) (300.0 mg; 1.074 mmol, 1.00 eq.) in MeOH (10 mL) added 10% Pd/C (30.0 mg; 10% w/w) under nitrogen atmosphere. The mixture was hydrogenated at room temperature overnight under hydrogen atmosphere (2 atm). The desired product was detected via LCMS. The resulting mixture was filtered, the filter cake was washed with MeOH (3×5 mL). The filtrate was concentrated under reduced pressure. The residue was purified by flash chromatography on silica gel column using 10-90% of EtOAc in petroleum ether as eluent to provide (1r,3r)-3-(4-hydroxyphenoxy)cyclobutane-1-carbonitrile as an off-white solid (185.0 mg, 91.0%). LCMS (ESI) m/z 188.1, [M−H]⁻.

### Step 2: (1R,3R)-3-(4-hydroxy-3-nitrophenoxy)cyclobutane-1-carbonitrile

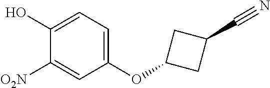 [SMILES: N#C[C@H]1C[C@H](Oc2ccc(O)c([N+](=O)[O-])c2)C1]

To a stilling mixture of (1R,3R)-3-(4-hydroxyphenoxy)cyclobutane-1-carbonitrile (180.0 mg; 0.951 mmol; 1.00 eq.) in a mixture solvent of MeCN/water (19:1, 10 nit) was added urea nitrate (117.0 mg; 0.951 mmol, 1.00 eq.). The reaction mixture was stirred at 80° C. overnight. The desired product was detected via LCMS. The resulting mixture was concentrated under reduced pressure. The residue was purified by flash chromatography on silica gel column using 20-90% of EtOAc in petroleum ether as eluent to provide (1r,3r)-3-(4-hydroxy-3-nitrophenoxy)cyclobutane-1-carbonitrile as a yellow solid (116.0 mg, 52.0%). LCMS (ESI) m/z 233.1, [M−H]⁻.

### Step 3: (1R,3R)-3-(3-amino-4-hydroxyphenoxy)cyclobutane-1-carbonitrile

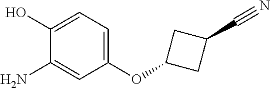 [SMILES: N#C[C@H]1C[C@H](Oc2ccc(O)c(N)c2)C1]

To a stirring mixture of (1R,3R)-3-(4-hydroxy-3-nitrophenoxy)cyclobutane-1-carbonitrile (116.0 mg; 0.495 mmol; 1.00 eq.) in MeOH (5 mL) was added 10% Pd/C (24.0 mg; 20% w/w) under nitrogen atmosphere. The mixture was hydrogenated at room temperature overnight under hydrogen atmosphere (2 atm). The desired product was detected via. LCMS. The resulting mixture was filtered, the filter cake was washed with MeOH (3×10 ML) and the filtrate was concentrated under reduced pressure. The residue was purified by flash chromatography on silica gel column using 50-90% of EtOAc in petroleum ether as eluent to provide (1R,3R)-3-(3-amino-4-hydroxyphenoxy)cyclobutane-1-carbonitrile as a yellow solid (63.0 mg, 62.2%). LCMS (ESI) m/z 205.1, [M+H]⁺.

### Step 4: N-(5-(5-((1R,3R)-3-cyanocyclobutoxy)benzo[d]oxazol-2-yl)-8-(methylamino)-2,7-naphthyridin-3-yl)cyclopropanecarboxamide

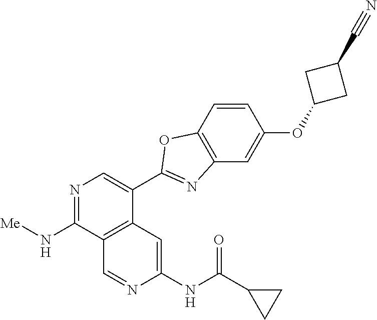 [SMILES: CNc1ncc(-c2nc3cc(O[C@H]4C[C@H](C#N)C4)ccc3o2)c2cc(NC(=O)C3CC3)ncc12]

N-(5-(5-((1R,3R)-3-cyanocyclobutoxy)benzo[d]oxazol-2-yl)-8-(methyl amino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and (1r,3r)-3-(3-amino-4-hydroxyphenoxy)cyclobutane-1-carbonitrile as the starting material. LCMS (ESE) m/z 455.2, [M+H]⁺. ¹H NMR (300 MHZ, DMSO-d₆) δ 11.04 (s, 1H), 9.57 (s, 1H), 9.43 (s, 1H), 8.88 (s, 1H), 8.68-8.62 (m, 1H), 7.63 (d, J=8.8 Hz, 1H), 7.16 (d, J=2.4 Hz, 1H), 6.91 (dd, J=8.8, 2.4 Hz, 1H), 4.88-4.75 (m, 1H), 3.16-3.10 (m, 1H), 3.09 (d, J=4.4 Hz, 3H), 3.03-2.91 (m, 2H), 2.46-2.31 (m, 2H), 2.14-2.06 (m, 0.93-0.83 (m, 4H).

### Example 234: Synthesis of N-(5-(5-(benzyloxy)benzo[d]oxazol-2-yl)-8-(methylamino)-2,7-naphthyridin-3-3-yl)cyclopropanecarboxamide

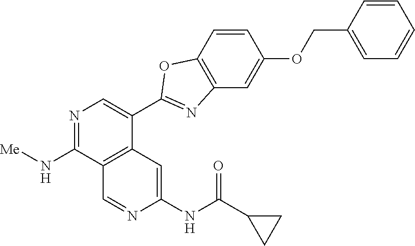 [SMILES: CNc1ncc(-c2nc3cc(OCc4ccccc4)ccc3o2)c2cc(NC(=O)C3CC3)ncc12]

### Step 1: 41-(benzyloxy)-2-nitrophenol

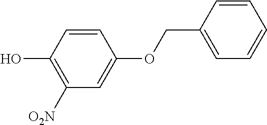 [SMILES: O=[N+]([O-])c1cc(OCc2ccccc2)ccc1O]

To a stirring mixture of 4-(benzyloxy)phenol (5.00 g; 24.970 mmol; 1.00 eq.) and urea nitrate (3.07 g; 24.970 mmol; 1.00 eq.) in a mixture solvent of MeCN/water (19:1, 100 mL) was reacted at 80° C. overnight. The desired product was detected via LCMS. The resulting mixture was concentrated under reduced pressure. The residue was purified by flash chromatography on silica gel column using 10-20% of EtOAc in petroleum ether as eluent to provide 4-(benzyloxy)-2-nitrophenol as a yellow solid (4.07 g, 66.1%). LCMS (ESI) m/z 246.1, [M+H]⁺.

### Step 2: 2-amino-4-(benzyloxy)phenol

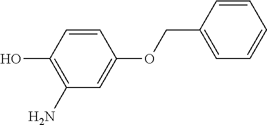 [SMILES: Nc1cc(OCc2ccccc2)ccc1O]

To a stirring mixture of 4-(benzyloxy)-2-nitrophenol (4.00 g; 16.31 mmol; 1.00 eq.) in EtOH/water (5:1, 120 mL) were added Fe (4.50 g; 80.58 mmol; 4.94 eq.) and NH₄Cl (6.98 g; 130.5 mmol; 8.00 eq.) at room temperature. The resulting solution was stirred at 80° C. for 2 hours. The desired product was detected via LCMS. The resulting mixture was filtered, and the filter cake was washed with EtOAc (5×20 mL). The filtrate was collected and concentrated under reduced pressure. The residue was purified by flash chromatography on silica gel column using 40-60% of EtOAc in petroleum ether as eluent to provide 2-amino-4-(benzyloxy)phenol as a yellow solid (2.00 g, 56.1%). LCMS (ESI) m/z 216.1, [M+H]⁺.

### Step 3: N-(5-(5-(benzyloxy)benzo[d]oxazol-2-yl)-8-(methylamino)-2,7-naphthyridin-3-yl)cyclopropanecarboxamide

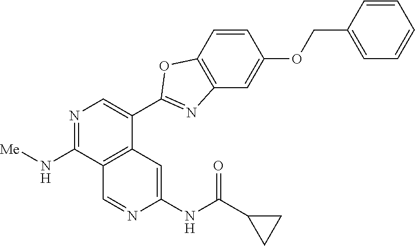 [SMILES: CNc1ncc(-c2nc3cc(OCc4ccccc4)ccc3o2)c2cc(NC(=O)C3CC3)ncc12]

N-(5-(5-(benzyloxy)benzo[d]oxazol-2-yl)-8-(methylamino)-2,7-naphthyridin-3-yl)cyclopropanecarboxamide was synthesized using a similar procedure that was previously, described in Example 210 by using N-(5-formyl-8-(methylamino)-2,7-naphthyridin-3-yl)cyclopropanecarboxamide and 2-amino-4-(benzyloxy)phenol as the starting material. LCMS (ESI) m/z 466.2, [M+H]⁺. ¹H NMR. (400 MHz, DMSO-d₆) δ 11.02 (s, 1H), 9.63 (s, 1H), 9.43 (s, 1H), 8.88 (s, 1H), 8.66-8.60 (m, 1H), 7.62 (d, J=8.8 Hz, 1H), 7.53-7.30 (m, 1H), 7.04 (dd, J=8.8, 2.4 Hz, 1H), 5.21 (s, 2H), 3.08 (d, J=4.4 Hz, 3H), 2.13-2.04 (m, 1H), 0.96-0.80 (m, 4H).

### Example 235: Synthesis of N-(8-(methylamino)-5-(5-(2-morpholinoethoxy)benzo[d]oxazol-2-yl)-2,7-naphthyridin-3-yl)cyclopropanecarboxamide

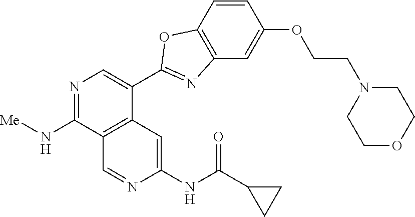 [SMILES: CNc1ncc(-c2nc3cc(OCCN4CCOCC4)ccc3o2)c2cc(NC(=O)C3CC3)ncc12]

### Step 1: 4-(2-(1-(benzyloxy)phenoxy)ethyl)morpholine

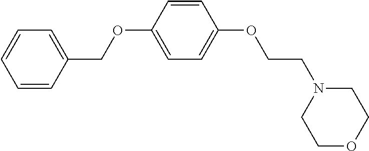 [SMILES: c1ccc(COc2ccc(OCCN3CCOCC3)cc2)cc1]

To a stirring mixture of 4-(benzyloxy)phenol (5.00 g; 24.970 mmol; 1.00 eq.) in DMF (60 mL) was added 4-(2-bromoethyl)morpholine hydrobromide (20.58 g; 74.910 mmol; 3.00 eq.) and Cs₂CO₃ (16.27 g; 49.940 mmol; 2.00 eq.) at room temperature. The reaction was stirred at 120° C. for 16 hours under nitrogen atmosphere. The desired product was detected via LCMS. The resulting mixture was concentrated under vacuum. The residue was purified by flash chromatography on silica gel column using 5-30% of EtOAc in petroleum ether as eluent to provide 4-(2-(4-(benzyloxy)phenoxy)ethyl)morpholine as a yellow oil (2.28 g, 29.1%). LCMS (ESI) m/z 314.2, [M+H]⁺.

### Step 2: 4-(2-morpholinoethoxy)phenol

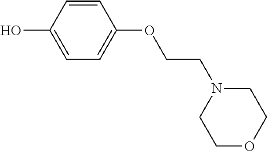 [SMILES: Oc1ccc(OCCN2CCOCC2)cc1]

To a stirring mixture of 4-(2-(4-(benzyloxy)phenoxy)ethyl)morpholine (2.28 g; 7.275 mmol; 1.00 eq.) in MeOH (80 mL) was added 10% Pd/C (684.0 mg; 30% w/w) under nitrogen atmosphere. The mixture was hydrogenated at room temperature for 3 hours under hydrogen atmosphere (2 atm). After filtration, the filtrate was concentrated under reduced pressure to afford 4-(2-morpholinoethoxy)phenol as brown solid (1.68 g, crude). LCMS (ESI) m/z 224.1 [M+H]⁺.

### Step 3: 4-(2-morpholinoethoxy)-2-nitrophenol

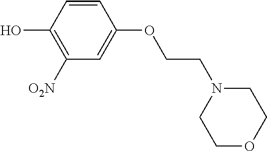 [SMILES: O=[N+]([O-])c1cc(OCCN2CCOCC2)ccc1O]

To a stirring mixture of 4-(2-morpholinoethoxy)phenol (1.68 g; 7,524 mmol; 1.00 eq.) in MeCN/water (5:1, 10 mL) was added urea nitrate (1.85 g; 15.048 mmol; 2.00 eq.) under nitrogen atmosphere. The reaction mixture was stirred at 100° C. for 1 hour under a microwave irradiation condition. The desired product was detected via LCMS. The reaction was diluted with EtOAc (400 mL) and washed with brine (3×10 mL). The organic phase was dried with Na₂SO₄, filtered, and concentrated under reduced pressure. The residue was purified by flash chromatography on silica gel column using 20-50% of EtOAc in petroleum ether as eluent to provide 4-(2-morpholinoethoxy)-2-nitrophenol as orange solid (346.0 mg, 17.1%). LCMS (ESI) m/z 269.1, [M+H]⁺.

### Step 4: 2-amino-4-(2-morpholinoethoxy)phenol

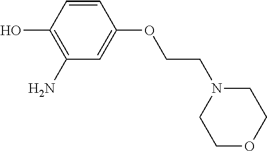 [SMILES: Nc1cc(OCCN2CCOCC2)ccc1O]

To a stirring mixture of 4-(2-morpholinoethoxy)-2-nitrophenol (200.0 mg; 0.746 mmol; 1.00 eq.) in MeOH (40 mL) was added 10% Pd/C (200.0 mg; 100% w/w) under nitrogen atmosphere. The mixture was hydrogenated at room temperature for 3 hours under hydrogen atmosphere (2 atm). After filtration, the filtrate was concentrated under reduced pressure to afford 2-amino-4-(2-morpholinoethoxy)phenol as brown solid (168.0 mg, crude). LCMS (ESI) m/z 239.1, [M+H]⁺.

### Step 5: N-(8-(methylamino)-5-(5-(2-morpholinoethoxy)benzo[d]oxazol-2-yl)-2,7-naphthyridin-3-yl)cyclopropanecarboxamide

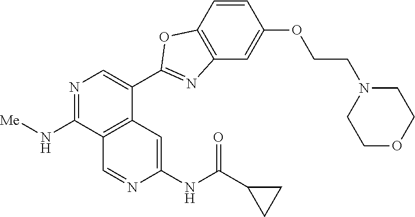 [SMILES: CNc1ncc(-c2nc3cc(OCCN4CCOCC4)ccc3o2)c2cc(NC(=O)C3CC3)ncc12]

N-(8-(methylamino)-5-(5-(2-morpholinoethoxy)benzo[d]oxazol-2-yl)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(2-morpholinoethoxy)phenol as the starting material. LCMS (ESI) m/z 489.2, [M+H]⁺. 1 H NMR (400 MHz, DMSO-d₆) δ 11.04 (s, 1H), 9.65 (5, 1H), 9.44 (s, 1H), 8.90 (s, 1H), 8.68-8.62 (m, 1H), 7.61 (d, J=8.8 Hz, 1H), 7.31 (d, J=2.4 Hz, 1H), 7.00-6.93 (m, 1H), 4.19 (t, 1=5.6 Hz, 2H), 3.64-3.57 (m, 4H), 3.09 (d, J=4.4 Hz, 3H), 2.74 (t, J=5.6 Hz, 2H), 2.61-2.52 (m, 4H), 2.16-2.05 (tri, 1H), 0.96-0.82 (m, 4H).

### Example 236: Synthesis of N-(5-(5-(2-cyano-2-methylpropoxy)benzo[d]oxazol-2-yl)-8-(methylamino)-2,7-naphthyridin-3-yl)cyclopropanecarboxamide

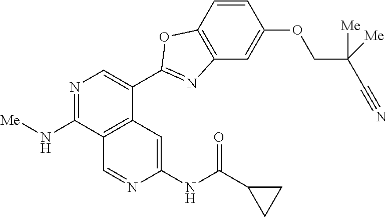 [SMILES: CNc1ncc(-c2nc3cc(OCC(C)(C)C#N)ccc3o2)c2cc(NC(=O)C3CC3)ncc12]

### Step 1: 2-cyano-2-methylpropyl ethanesulfonate

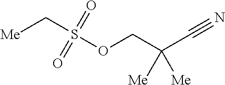 [SMILES: CCS(=O)(=O)OCC(C)(C)C#N]

To a stirring mixture of 3-hydroxy-2,2-di methylpropanenitrile (1.00 g; 10.087 mmol; 1.00 eq.) and Et₃N (3.06 g; 30.261 mmol; 3.00 eq.) in CH₂Cl₂ (40 mL) was added ethanesulfonyl chloride (1.94 g; 15.130 mmol; 1.5 eq.) dropwise at 0° C. The resulting mixture was stirred at room temperature for 2 hours under nitrogen atmosphere. The desired product was detected via TLC. The resulting mixture was diluted with CH₂Cl₂ (100 mL) and washed with brine (3×10 mL). The organic phase was concentrated under vacuum to afford 2-cyano-2-methylpropyl ethanesulfonate as a off-white oil (1.50 g, crude). The crude product was used in the next step directly without further purification.

### Step 2: 3-(4-(benzyloxy)phenoxy)-2,2-dimethylpropanenitrile

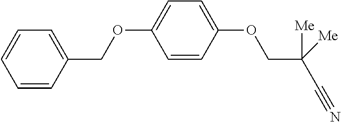 [SMILES: CC(C)(C#N)COc1ccc(OCc2ccccc2)cc1]

A stirring mixture of 2-cyano-2,2-dimethylethyl ethanesulfonate (1.50 g; 7.843 mmol; 1.00 eq.), 4-(benzyloxy)phenol (1.88 g; 9.412 mmol; 1.20 eq.) and Cs₂CO₃ (7.66 g; 23.529 mmol; 3.00 eq.) in DMF (8 mL) was stirred at 85° C. for 14 hours. The desired product was detected via LCMS. The resulting mixture was diluted with EtOAc (100 mL) and washed with brine (3×10 mL). The organic phase was concentrated under vacuum. The residue was purified by flash chromatography on pre-packed C18 column using 60-70% of MeCN in water (10 mmol/L NH₄HCO₃) as eluent to provide 3-(4-(benzyloxy)phenoxy)-2,2-dimethylpropanenitrile as a white solid (600.0 mg, 27.1%). LCMS (ESI) m/z 280.1, [M+H]⁺.

### Step 3: 3-(4-hydroxyphenoxy)-2,2-dimethylpropanenitrile

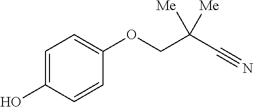 [SMILES: CC(C)(C#N)COc1ccc(O)cc1]

To a stirring mixture of 3-(4-(benzyloxy)phenoxy)-2,2-dimethylpropanenitrile (300.0 mg; 1.10 mmol; 1.00 eq.) in Maki, (30 mL) was added 10% Pd/C (60.0 mg; 20% w/w) under nitrogen atmosphere. The mixture was hydrogenated at room temperature for 14 hours under hydrogen atmosphere (2 atm). The desired product was detected via LCMS. After filtration, the filtrate was concentrated under reduced pressure. The residue was purified by flash chromatography on pre-packed C18 column using 40-70% of MeCN in water (10 mmol/L NH₄HCO₃) as eluent to provide 3-(4-hydroxyphenoxy)-2,2-dimethylpropanenitrile as a white solid (150.0 mg, 73.5%). LCMS (ESI) m/z 190.1, [M−H]⁻.

### Step 4: 3-(4-hydroxy-3-nitrophenoxy)-2,2-dimethylpropanenitrile

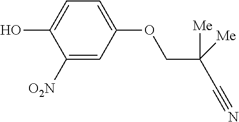 [SMILES: CC(C)(C#N)COc1ccc(O)c([N+](=O)[O-])c1]

A stirring mixture of 3-(4-hydroxyphenoxy)-2,2-dimethylpropanenitrile (200.0 mg; 1.046 mmol; 1.00 eq.) and urea nitrate (128.7 mg; 1.046 mmol; 1.00 eq.) in MeCN/water (19:1, 5 mL) was stirred at 80° C. for 14 hours. The desired product was detected via LCMS. The solvent was concentrated under vacuum. The residue was purified by flash chromatography on pre-packed C18 column using 50-70% of MeCN in water (10 mmol/L NH₄HCO₃) as eluent to provide 3-(4-hydroxy-3-nitrophenoxy)-2,2-dimethylpropanenitrile as a black solid (170.0 mg, 68.7%). LCMS (ESI) m/z 235.1, [M−H]⁻.

### Step 5: 3-(3-amino-4-hydroxyphenoxy)-2,2-dimethylpropanenitrile

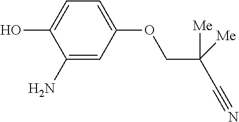 [SMILES: CC(C)(C#N)COc1ccc(O)c(N)c1]

To a stirring mixture of 3-(4-hydroxy-3-nitrophenoxy)-2,2-dimethylpropanenitrile (170.0 mg; 0.720 mmol; 1.00 eq.) in MeOH (10 mL) was added 10% Pd/C (34.0 mg; 20% w/w) under nitrogen atmosphere. The mixture was hydrogenated at room temperature for 12 hours under hydrogen atmosphere (2 atm). The desired product was detected via LCMS. After-filtration, the filtrate was concentrated under reduced pressure. The residue was purified by flash chromatography on pre-packed C18 column using 60-70% of MeCN in water (10 mmol/L NH₄HCO₃) as eluent to 3-(3-amino-4-hydroxyphenoxy)-2,2-dimethylpropanenitrile as a black solid (130.0 mg, 87.5%). LCMS (ESI) m/z 207.1, [M+H]⁺.

### Step 6: N-(5-(5-(2-cyano-2-methylpropoxy)benzo[d]oxazol-2-yl)-8-(methylamino)-2,7-naphthyridin-3-yl)cyclopropanecarboxamide

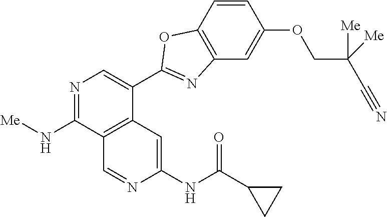 [SMILES: CNc1ncc(-c2nc3cc(OCC(C)(C)C#N)ccc3o2)c2cc(NC(=O)C3CC3)ncc12]

N-(5-(5-(2-cyano-2-methylpropoxy)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 3-(3-amino-4-hydroxyphenoxy)-2,2-dimethylpropanenitrile as the starting material. LCMS (ESI) m/z 457.2, [M+H]⁺. ¹H NMR (300 MHz, DMSO-d₆) δ 11.05 (s, 1H), 9.68 (s, 1H), 9.44 (s, 1H), 8.91 (s, 1H), 8.70-8.61 (m, 1H), 7.65 (d, J=8.8 Hz, 1H), 7.35 (d, J=2.4 Hz, 1H), 7.03 (dd, J=8.8, 2.4 Hz, 1H), 4.12 (5, 2H), 3.09 (d, J=4.4 Hz, 3H), 2.14-2.07 (m, 1H), 1.46 (s, 6H), 0.94-0.81 (m, 4H).

### Example 237: Synthesis of 2-(6-(cyclopropanecarboxamido naphthyridin-4-yl)benzo[d]oxazole-5-carboxamide

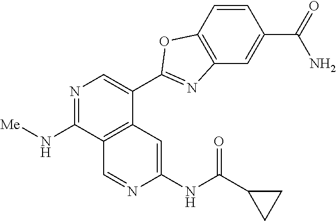 [SMILES: CNc1ncc(-c2nc3cc(C(N)=O)ccc3o2)c2cc(NC(=O)C3CC3)ncc12]

To a stirring mixture of N-(5-(5-cyanobenzo[d]oxazol-2-yl)-8-(methylamino)-2,7-naphthyridin-3-yl)cyclopropanecarboxamide (Example 210) (50.0 mg 0.130 mmol; 1.00 eq.) and K₂CO₃ (53.9 mg; 0.390 mmol; 3.00 eq.) in DMSO (3 mL) was added H₂O₂ (30% in water, 0.5 mL) at 0° C. The resulting solution was stirred at room temperature for 5 hours under nitrogen atmosphere. The reaction was quenched by a saturated Na₂SO₃ aqueous solution (2 mL). The mixture was purified by flash chromatography on pre-packed. C18 column using 30-100% of MeOH in water (10 mmol/L NH₄HCO₃) to provide 2-(6-(cyclopropanecarboxamido)-1-(methylamino)-2,7-naphthyridin-4-yl)benzo[d]oxazole-5-carboxamide as a light yellow solid (12.6 mg, 23.8%). LCMS (ESI) m/z 403.1, [M+H]⁺. ¹H NMR (400 MHz, DMSO-d₆) δ 11.05 (s, 1H), 9.74 (s, 1H), 9.45 (s, 1H), 8.95 (s, 1H), 8.80-8.60 (m, 1H), 8.30 (d, J=1.6 Hz, 1H), 8.16 (s, 1H), 7.96 (dd, J=8.4, 1.6 Hz, 1H), 7.78 (d, J=8.4 Hz, 1H), 7.41 (s, 1H), 3.10 (d, J=4.4 Hz, 3H), 2.18-2.03 (m, 1H), 0.99-0.80 (m, 4H).

### Example 238: Synthesis of 2-(6-(cyclopropanecarboxamido)-1-(methylamino)-2,7-naphthyridin-4-yl)-N-methylbenzo[d]oxazole-5-carboxamide

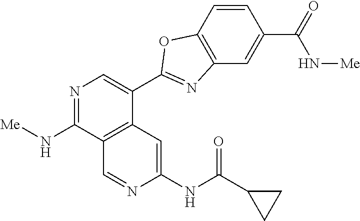 [SMILES: CNC(=O)c1ccc2oc(-c3cnc(NC)c4cnc(NC(=O)C5CC5)cc34)nc2c1]

To a stirring mixture of 2-(6-(cyclopropanecarboxamido)-1-(methylamino)-2,7-naphthyridin-4-yl)benzo[d]oxazole-5-carboxylic acid (60.0 mg; 0.149 mmol; 1.00 eq.) in DMF (4 mL) was added methanamine hydrochloride (40.1 mg; 0.596 mmol; 4.00 eq.), HATU (169.6 mg; 0.447 mmol, 3.00 eq.) and DIPEA (99.3 mg; 0.770 mmol; 5.00 eq). The resulting mixture was stirred at room temperature for 14 hours. The desired product was detected via LCMS. The resulting mixture was purified by flash chromatography on pre-packed C18 column using 50-90% of MeOH in water (10 NH₄HCO₃) as eluent to provide 2-(6-(cyclopropanecarboxamido)-1-(methylamino)-2,7-naphthyridin-4-yl)-N-methylbenzo[d]oxazole-5-carboxamide as a yellow brown solid (35.8 mg, 55.3%). (ESI) m/z 417.2, [M+H]⁺. ¹H NMR (400 MHz, DMSO-d₆) δ 11.05 (s, 1H), 9.76 (s, 1H), 9.45 (s, 1H), 8.95 (s, 1H), 8.73-8.69 (m, 1H), 8.64-8.61 (m, 1H), 8.25 (d, J=2.4 Hz, 1H), 7.92 (dd, J=8.8, 2.4 Hz, 1H), 7.79 (d, J=8.8 Hz, 1H), 3.10 (d, J=4.4 Hz, 3H), 2.83 (d, J=4.8 Hz, 3H), 2.14-2.07 (m, 1H), 0.94-0.84 (m, 4H).

### Example 239: Synthesis of (R)—N-(5-(5-(2-hydroxy-propoxy)benzo(methylamino)-2,7-naphthyridin-3-yl)cyclopropanecarboxamide

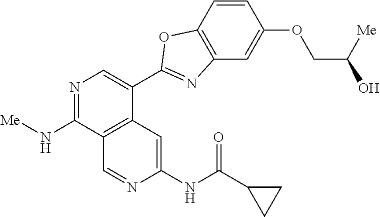 [SMILES: CNc1ncc(-c2nc3cc(OC[C@@H](C)O)ccc3o2)c2cc(NC(=O)C3CC3)ncc12]

### Step 1: (R)-1-(4-(benzyloxy)phenoxy)propan-2-al

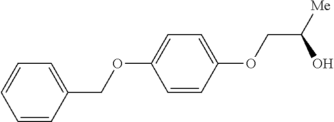 [SMILES: C[C@@H](O)COc1ccc(OCc2ccccc2)cc1]

A mixture of 4-(benzyloxy)phenol (1.00 g; 4.994 mmol; 1.00 eq.) and (R)-2-methyloxirane (348.0 mg; 5.993 mmol; 1.20 eq.), NaOH (99.8 mg; 0.250 mmol, 0.05 eq.) in water (8 mL) was stirred at 50° C. overnight under nitrogen atmosphere. The desired product was detected via LCMS. The resulting mixture was extracted with CH₂Cl₂ (3×20 mL). The combined organic layers were dried over anhydrous Na₂SO₄. After filtration, the filtrate was concentrated under reduced pressure. The residue was purified by flash chromatography on silica gel column using 30-60% of EtOAc in petroleum ether as eluent to provide (R)-1-(4-(benzyloxy)phenoxy)propan-2-ol as a white solid. (1.00 g, 77.5%). LCMS (ESI) m/z 257.1. [M−H]⁻.

### Step 2: (R)-4-(2-hydroxypropoxy)phenol

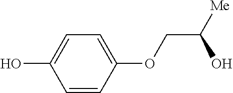 [SMILES: C[C@@H](O)COc1ccc(O)cc1]

To a stirring mixture of (R)-1-(4-(benzyloxy)phenoxy)propan-2-ol (800.0 mg; 3.097 mmol; 1.00 eq.) in MeOH (10 mL) was added 10% Pd/C (160.0 mg; 20% w/w) under nitrogen atmosphere. The mixture was hydrogenated at room temperature overnight under hydrogen atmosphere (2 atm). The desired product was detected via LCMS. After filtration, the filtrate was concentrated under reduced pressure. The residue was purified by flash chromatography on pre-packed. C18 column using 40-60% of MeCN in water (10 mmol/L, NH₄HCO₃) as eluent to (R)-4-(2-hydroxypropoxy)phenol as a white solid (450.0 mg, 86.3%). LCMS (ESI) m/z 167.1, [M−H]⁻.

### Step 3: (R)-4-(2-hydroxypropoxy)-2-nitrophenol

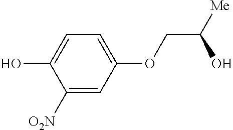 [SMILES: C[C@@H](O)COc1ccc(O)c([N+](=O)[O-])c1]

To a stirring mixture of (R)-4-(2-hydroxypropoxy)phenol (350.0 mg; 2.081 mmol; 1.00 eq.) and urea nitrate (256.1 mg; 2.081 mmol, 1.00 eq.) in MeCN/water (19:1, 5 mL) was stirred at 80° C. for 14 hours. The desired product was detected via LCMS. The solvent was concentrated under vacuum. The residue was purified by flash chromatography on pre-packed C18 column using 10-40% of MeCN in water (10 mmol/L NH₄CO₃) as eluent to provide (R)-4-(2-hydroxypropoxy)-2-nitrophenol as a black solid (200.0 mg, 45.1%). LCMS (ESI) m/z 212.1, [M−H]⁻.

### Step 4: (R)-2-amino-4-(2-hydroxypropoxy)phenol

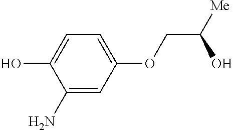 [SMILES: C[C@@H](O)COc1ccc(O)c(N)c1]

To a stirring mixture of (R)-4-(2-hydroxypropoxy)-2-nitrophenol (200.0 mg; 0.938 mmol; 1.00 eq.) in MeOH (10 mL) was added 10% Pd/C (40.0 m; 20% w/w) under nitrogen atmosphere. The mixture was hydrogenated at room temperature for 12 hours under hydrogen atmosphere (2 atm).

The desired product was detected via LCMS. After filtration, the filtrate was concentrated under reduced pressure. The residue was purified by flash chromatography on silica gel column using 10-90% of EtOAc in petroleum ether as eluent to provide (R)-2-amino-4-(2-hydroxypropoxy)phenol as a brown solid (100.0 mg, 58.1%). LCMS (ESI) m/z 184.1, [M+H]⁺.

### Step 5: (R)—N-(5-(5-(2-hydroxypropoxy)benzo[d]oxazol-2-yl)-8-(methylamino)-2,7-naphthyridin-3-yl)cyclopropanecarboxamide

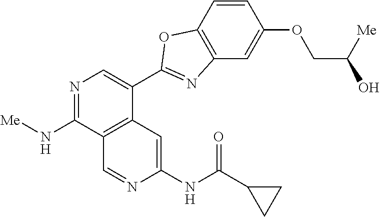 [SMILES: CNc1ncc(-c2nc3cc(OC[C@@H](C)O)ccc3o2)c2cc(NC(=O)C3CC3)ncc12]

(R)—N-(5-(5-(2-hydroxypropoxy)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and (R)-2-amino-4-(2-hydroxypropoxy)phenol as the starting material. LCMS (ESI) m/z 434.2, [M+H]⁺. ¹H NMR (300 MHz, DMSO-d₆) δ 11.04 (s, 1H), 9.65 (5, 1H), 9.43 (s, 1H), 8.89 (s, 1H), 8.68-8.63 (m, 1H), 7.61 (d, J=8.8 Hz, 1H), 7.26 (d, J=2.4 Hz, 1H), 6.96 (dd, J=8.8, 2.4 Hz, 1H), 4.90 (d, J=4.8 Hz, 1H), 4.04-3.87 (m, 3H), 3.08 (d, or 4.4 Hz, 3H), 2.14-2.06 (m, 1H), 1.18 (d, J=6.3 Hz, 3H), 0.95-0.81 (m, 4H).

### Example 240: Synthesis of (S)—N-(5-(5-(2-hydroxypropoxy)benzo[d]oxazol-2-yl)-8-(methylamino)-2,7-naphthyridin-3-yl)cyclopropanecarboxamide

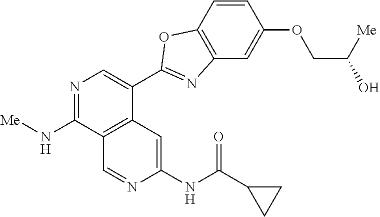 [SMILES: CNc1ncc(-c2nc3cc(OC[C@H](C)O)ccc3o2)c2cc(NC(=O)C3CC3)ncc12]

### Step 1: (S)-1-(4-(benzyloxy)phenoxy)propan-2-ol

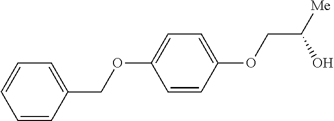 [SMILES: C[C@H](O)COc1ccc(OCc2ccccc2)cc1]

To a stirring mixture of 4-(benzyloxy)phenol (1.00 g; 4.994 mmol; 1.00 eq.), (S)-2-methyloxirane (348.0 mg; 5.993 mmol; 1.20 eq.) in water (8 mL) was added NaOH (99.8 mg; 0.25 mmol; 0.05 eq.) was stirred at 50° C. for 14 h under nitrogen atmosphere. Upon completion, the resulting mixture was extracted with CH₂Cl₂ (3×20 mL). The combined organic layers were dried over anhydrous Na₂SO₄, filtered, and concentrated under reduced pressure. The residue was purified via a silica gel column using 30-60% of EtOAc in petroleum ether as eluent to provide (S)-1-(4-(benzyloxy)phenoxy)propan-2-ol as a white solid (850 mg; 65.8%). LCMS (ESI) m/z 257.1, [M−H]⁻.

### Step 2: (S)-4-(2-hydroxypropoxy)phenol

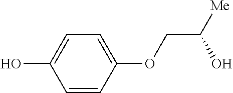 [SMILES: C[C@H](O)COc1ccc(O)cc1]

To a stirring mixture of (S)-1-(4-(benzyloxy)phenoxy)propan-2-ol (750.0 mg; 2.903 mmol; 1.00 eq.) in MeOH (10 mL) was added 10% Pd/C (150.0 mg; 20% w/w) under nitrogen atmosphere. The mixture was hydrogenated at room temperature for 12 hours under hydrogen atmosphere (2 atm). The desired product was detected via LCMS. After filtration, the filtrate was concentrated under reduced pressure. The residue was purified by flash chromatography on silica gel column using 10-60% of EtOAc in petroleum ether as eluent to provide (S)-4-(2-hydroxypropoxy)phenol as a white solid (400.0 mg, 81.9%). LCMS (ESI) m/z 167.1, [M−H]⁻.

### Step 3: (S)-4-(2-hydroxypropoxy)-2-nitrophenol

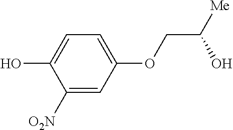 [SMILES: C[C@H](O)COc1ccc(O)c([N+](=O)[O-])c1]

To a stirring mixture of (S)-4-(2-hydroxypropoxy)phenol (350.0 mg; 2.081 mmol; 1.00 eq.) in a mixture solvent of MeCN/water (19:1, 5 mL) was added urea nitrate (256.1 mg; 2.081 mmol; 1.00 eq.). The resulting mixture was stirred at 80° C. for 14 hours. The desired product was detected via. LCMS. The solvent was concentrated under vacuum. The residue was purified by flash chromatography on pre-packed C18 column using 10-40% of MeCN in water (10 mmol/L NH₄HCO₃) as eluent to provide (S)-4-(2-hydroxypropoxy)-2-nitrophenol as a black solid (220.0 mg, 49.5%). LCMS (ESI) m/z 212.1, [M−H]⁻.

### Step 4: (S)-2-amino-4-(2-hydroxypropoxy)phenol

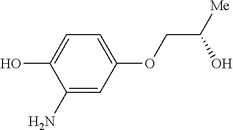 [SMILES: C[C@H](O)COc1ccc(O)c(N)c1]

To a stirring mixture of (S)-4-(2-hydroxypropoxy)-2-nitrophenol (220.0 mg; 1.032 mmol; 1.00 eq.) in MeOH (10 mL) was added 10% Pd/C (44.0 mg; 20% w/w) under nitrogen atmosphere. The mixture was hydrogenated at room temperature for 14 hours under hydrogen atmosphere (2 atm). The desired product was detected via LCMS. After filtration, the filtrate was concentrated under reduced pressure. The residue was purified by flash chromatography on silica gel column using 10-90% of EtOAc in petroleum ether as eluent to provide (S)-2-amino-4-(2 as a brown solid (120.0 mg, 63.4%). LCMS (ESI) m/z 184.1, [M+H]⁺.

### Step 5: (S)—N-(5-(5-(2-hydroxypropoxy)benzo[d]oxazol-2-yl)-8-(methylamino)-2,7-naphthyridin-3-yl)cyclopropanecarboxamide

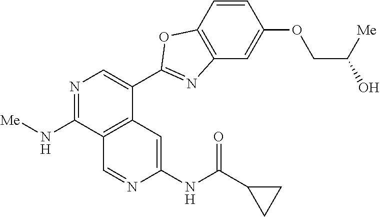 [SMILES: CNc1ncc(-c2nc3cc(OC[C@H](C)O)ccc3o2)c2cc(NC(=O)C3CC3)ncc12]

(S)—N-(5-(5-(2-hydroxypropoxy)benzo[d]oxazol-2-yl)-8-(methyl amino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and (S)-2-amino-4-(2-hydroxypropoxy)phenol as the starting material. LCMS (ESI) m/z 434.2, [M+H]⁺. ¹H NMR (300 MHz, DMSO-d₆) δ 11.04 (s, 1H), 9.65 (s, 1H), 9.43 (s, 1H), 8.89 (s, 1H), 8.68-8.63 (m, 1H), 7.61 (d, J=8.8 Hz, 1H), 7.26 (d, J=2.4 Hz, 1H), 6.96 (dd, J=8.8, 2.4 Hz, 1H), 4.90 (d, J=4.8 Hz, 1H), 4.04-3.87 (m, 3H), 3.08 (d, J=4.4 Hz, 3H), 2.14-2.06 (m, 1H), 1.18 (d, J=6.3 Hz, 3H), 0.95-0.81 (m, 4H).

### Example 241: Synthesis of (R)—N-(5-(5-(2-methoxypropoxy)benzo[d]oxazol-2-yl)-8-(methylamino)-2,7-naphthyridin-3-yl)cyclopropanecarboxamide

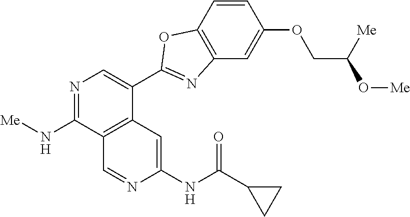 [SMILES: CNc1ncc(-c2nc3cc(OC[C@@H](C)OC)ccc3o2)c2cc(NC(=O)C3CC3)ncc12]

### Step 1: (R)-2-methoxypropyl methanesulfonate

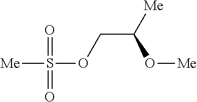

To a stirred solution of (R)-2-methoxypropan-1-ol (400.0 mg; 4.44 mmol; 1.00 eq.) and DIPEA (573.1 mg; 4.433 mmol; 1.00 eq.) in CH₂Cl₂ (8 mL) was added methanesulfonic anhydride (1.54 g; 8.875 mmol; 2.00 eq.) dropwise at 0° C. under nitrogen atmosphere. The resulting mixture was stirred for 2 hours at room temperature. The reaction was quenched by a saturated sodium bicarbonate (10 mH) solution, extracted with CH₂Cl₂. (3×30 mL). The combined organic layers were washed with brine (2×5 mL), dried over Na₂SO₄. After filtration, the filtrate was concentrated under reduced pressure to afford (R)-2-methoxypropyl methanesulfonate as a brown solid (660 mg, crude).

### Step 2: (R)-1-(benzyloxy)-4-(2-methoxypropoxy)benzene

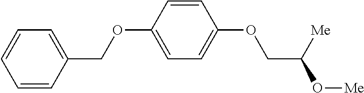 [SMILES: CO[C@H](C)COc1ccc(OCc2ccccc2)cc1]

To a stirring mixture of (R)-2-methoxypropyl methanesulfonate (660.0 mg; 3.924 mmol; 1.00 eq.) in DMF (3 mL) was added 4-(benzyloxy)phenol (392.1 mg; 1.958 mmol; 0.50 eq.) was added Cs₂CO₃ (1.92 g; 5.896 mmol; 1.50 eq.). The resulting solution was stirred at 100° C. overnight under nitrogen atmosphere. The resulting mixture was diluted with water (10 mL), extracted with EtOAc (3×30 mL). The combined organic layers were washed with brine (3×10 mL), dried over anhydrous Na₂SO₄, filtered; and concentrated under reduced pressure. The residue was purified by flash chromatography on silica gel column using 0-10% of MeOH in CH₂Cl₂ as eluent to provide (R)-1-(benzyloxy)-4-(2-methoxypropoxy)benzene as a yellow solid (430.1 mg, 40.2%). LCMS (ESI) m/z 273.1, [M+H]⁺.

### Step 3: (R)-4-(2-methoxypropoxy)phenol

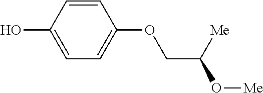 [SMILES: CO[C@H](C)COc1ccc(O)cc1]

To a solution of (R)-1-(benzyloxy)-4-(2-methoxypropoxy)benzene (430.1 mg; 1.579 mmol; 1.00 eq.) in MeOH (10 mL) was added 10% Pd/C (129.0 mg; 30% w/w) under nitrogen atmosphere. The mixture was hydrogenated at room temperature for 3 hours under hydrogen atmosphere (2 atm). After filtration, the filtrate was concentrated under reduced pressure. The residue was purified by flash chromatography on silica gel column using 15-50% of MeOH in CH₂Cl₂ as eluent to provide (R)-4-(2-methoxypropoxy)phenol as a yellow solid (280.0 mg, 97.3%), LCMS (ESI) m/z 183.1, [M+H]⁺.

### Step 4: (R)-4-(2-methoxypropoxy)-nitrophenol

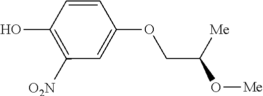 [SMILES: CO[C@H](C)COc1ccc(O)c([N+](=O)[O-])c1]

To a stirring mixture of (R)-4-(2-methoxypropoxy)phenol (200.0 mg; 1.098 mmol, 1.00 eq.) in a mixture solvent of MeCN/water (20:1, 4.2 mL) was added urea nitrate (134.0 mg; 1.089 mmol, 1.00 eq.). The mixture was stirred at 80° C. for 1 hour under nitrogen atmosphere. The resulting mixture was diluted with water (10 mL) and extracted with EtOAc (3×30 mL). The combined organic layers were washed with brine (3×10 mL), dried over anhydrous Na₂SO₄. After filtration, the filtrate was concentrated under reduced pressure. The residue was purified via a flask chromatography using 0-10% of MeOH in CH₂Cl₂ as eluent to provide (R)-4-(2-methoxypropoxy)-2-nitrophenol as a yellow liquid (250.1 mg, 90.0%). LCMS (ESI) m/z 228.1, [M+H]⁺.

### Step 5: (R)-2-amino-4-(2-methoxypropoxy)phenol

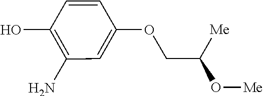 [SMILES: CO[C@H](C)COc1ccc(O)c(N)c1]

To a stirring mixture of (R)-4-(2-methoxypropoxy)-2-nitrophenol (110.1 mg; 0.484 mmol; 1.00 eq.) in MeOH (5 mL) was added 10% Pd/C (110.1 mg; 100% w/w) under nitrogen atmosphere. The mixture was hydrogenated at room temperature for 3 hours under hydrogen atmosphere (2 atm). After filtration, the filtrate was concentrated under reduced pressure to afford (R)-2-amino-4-(2-methoxypropoxy)phenol as a, yellow solid (81.2 mg, crude), LCMS (ESI) m/z 1.98, [M+H]⁺.

### Step 6: (R)—N-(5-(5-(2-methoxypropoxy)benzo[d]oxazol-2-yl)-8-(methylamino)-2,7-naphthyridin-3-yl)cyclopropanecarboxamide

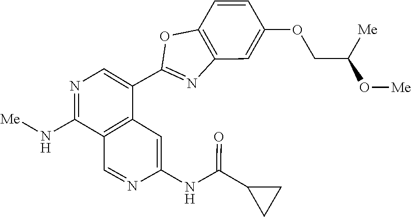 [SMILES: CNc1ncc(-c2nc3cc(OC[C@@H](C)OC)ccc3o2)c2cc(NC(=O)C3CC3)ncc12]

(R)—N-(5-(5-(2-methoxypropoxy)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and (R)-2-amino-4-(2-methoxypropoxy)phenol as the starting material. LCMS (ESI) m/z 448.2, [M+H]⁺. ¹H NMR (400 MHz, DMSO-d₆) δ 11.03 (s, 1H), 9.65 (s, 1H), 9.44 (s, 1H), 8.90 (s, 1H), 8.72-8.53 (m, 1H), 7.62 (d, J=8.8 Hz, 1H), 7.29 (d, J=2.4 Hz, 1H), 7.08-6.80 (m, 1H), 4.04 (d, J=4.8 Hz, 2H), 3.80-3.65 (m, 1H), 3.35 (s, 3H), 3.09 (d, J=4.4 Hz, 3H), 2.19-2.03 (m, 1H), 1.22 (d, J=6.4 Hz, 3H), 0.99-0.76 (m, 4H).

### Example 242: Synthesis of (S)—N-(5-(5-(2-methoxypropoxy)benzo[d]oxazol-2-yl)-8-(methylamino)-2,7-naphthyridin-3-yl)cyclopropanecarboxamide

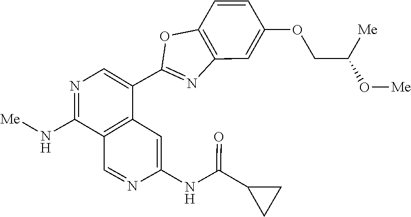 [SMILES: CNc1ncc(-c2nc3cc(OC[C@H](C)OC)ccc3o2)c2cc(NC(=O)C3CC3)ncc12]

### Step 1: (S)-2-methoxypropyl methanesulfonate

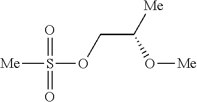

To a stirring mixture of (S)-2-methoxypropan-1-ol (500.0 mg; 5.548 mmol; 1.00 eq.) and DIEA (717.0 mg; 5.548 mmol; 1.00 eq.) in CH₂Cl₂ (12 mL) was added methanesulfonic anhydride (1.93 g; 11.096 mmol; 2.00 eq.) at 0° C. under nitrogen atmosphere. The reaction was stirred at room temperature for 2 hours. The reaction was quenched with a saturated NH₄HCO₃ solution (5 mL). The resulting mixture was diluted with CH₂Cl₂ (100 mL), washed by brine (3×10 mL), dried over anhydrous sodium sulfate, filtered, and concentrated under reduced pressure to afford (S)-2-methoxypropyl methanesulfonate as white oil (957.0 mg, crude).

### Step 2: (8)-1-(benzyloxy)-4-(2-methoxypropoxy)benzene

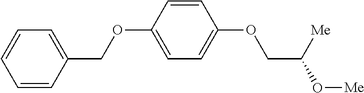 [SMILES: CO[C@@H](C)COc1ccc(OCc2ccccc2)cc1]

To a solution of (S)-2-methoxypropyl methanesulfonate (650.0 mg; 3.864 mmol; 2.00 eq.) in DMF (10 mL) was added 4-(benzyloxy)phenol (386.8 mg; 1.932=lot; 1.00 eq.) and Cs₂CO₃ (1.87 g; 5.757 mmol; 3.00 eq.) under nitrogen atmosphere. The reaction was stirred at 100° C. for 16 hours. Upon completion, the resulting mixture was diluted with EtoAc (200 mL) and washed by brine (3×20 mL). The organic layer was dried over anhydrous sodium sulfate and concentrated under vacuum. The residue was purified via a silica gel column using 10-60% of EtOAc in petroleum ether as eluent to provide (S)-1-(benzyloxy)-4-(2-methoxypropoxy)benzene as white oil (470.0 mg, 44.7%). LCMS (ESI) m/z 273.1, [M+H]⁺.

### Step 3: (S)-4-(2-methoxypropoxy)phenol

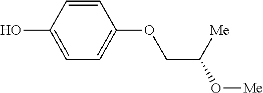 [SMILES: CO[C@@H](C)COc1ccc(O)cc1]

To a stirring mixture of (S)-1-(benzyloxy)-4-(2-methoxypropoxy)/benzene (470.0 mg; 1.666 mmol; 1.00 eq.) in MeOH (10 mL) was added 10% Pd/C (470.0 mg; 100% w/w) under nitrogen atmosphere. The mixture was hydrogenated at 40° C. for 3 hours under hydrogen atmosphere (2 atm). Upon completion, the filtrate was concentrated under reduced pressure. The residue was purified by flash chromatography on pre-packed C18 column using 20-60% of ACID in water (10 mmol/L, NH₄HCO₃) as eluent to provide (S)-4-(2-methoxypropoxy)phenol as white solid (295.0 mg, 93.9%).

### Step 4: (S)-4-(2-methoxypropoxy)-2-nitrophenol

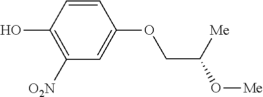 [SMILES: CO[C@@H](C)COc1ccc(O)c([N+](=O)[O-])c1]

To a solution of (S)-4-(2-methoxypropoxy)phenol (280.0 mg; 1.537 mmol; 1.00 eq.) in a mixture solvent of MeCN/water (20:1, 10 mL) was added urea nitrate (378.1 mg; 3.074 mmol; 2.00 eq.) at 0° C. under nitrogen atmosphere. The reaction was stirred at 80° C. for 1 hour. The resulting mixture was concentrated under reduced pressure. The residue was purified by flash chromatography on silica gel column using 10-40% of EtOAc in petroleum ether as eluent to provide (S)-4-(2-methoxypropoxy)-2-nitrophenol as white oil (192.0 mg, 54.9%). LCMS (ESI) m/z 228.1, [M+H]⁺.

### Step 5: (S)-2-amino-4-(2-methoxypropoxy)phenol

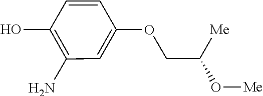 [SMILES: CO[C@@H](C)COc1ccc(O)c(N)c1]

To a stirring mixture of (8)-4-(2-methoxypropoxy)-2-nitrophenol (192.0 mg 0.845 mmol; 1.00 eq.) in MeOH (10 mL) added 10% Pd/C (192.0 mg; 100% w/w) under nitrogen atmosphere. The mixture was hydrogenated at 40° C. for 3 hours under hydrogen atmosphere (2 atm). The desired product was detected via After filtration, the filtrate was concentrated under reduced pressure to provide (S)-2-amino-4-(2-methoxypropoxy)phenol as white oil (114.0 mg, crude). LCMS (ESI) m/z 198.1, [M+H]⁺.

### Step 6: (S)—N-(5-(5-(2-methoxypropoxy)benzo[d]oxazol-2-yl)-8-(methylamino)-2,7-naphthyridin-3-yl)cyclopropanecarboxamide

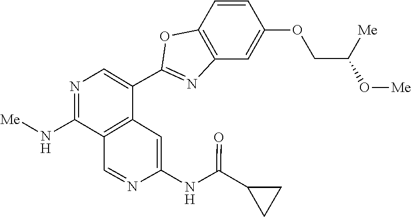 [SMILES: CNc1ncc(-c2nc3cc(OC[C@H](C)OC)ccc3o2)c2cc(NC(=O)C3CC3)ncc12]

(S)—N-(5-(5-(2-methoxy propoxy)benzo[d]oxazol-2-yl)-8-(methy amino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and (S)-2-amino-4-(2-methoxypropoxy)phenol as the starting material. LCMS (ESI) m/z 448.1, [M+H]⁺. ¹H NMR (400 MHz, DMSO-d₆) δ 11.03 (s, 1H), 9.65 (s, 1H), 9.44 (s, 1H), 8.90 (s, 1H), 8.68-8.61 (m, 1H), 7.62 (d, 8.8 Hz, 1H), 7.29 (d, J=2.4 Hz, 1H), 6.97 (d, J=8.8, 2.4 Hz, 1H), 4.04 (d, J=5.2 Hz, 2H), 3.77-3.65 (m, 1H), 3.32 (s, 3H), 3.09 (d, J===4.4 Hz, 3H), 2.15-2.05 (m, 1H), 1.22 (d, J=6.4 Hz, 3H), 0.96-0.82 (m, 4H).

### Example 243: Synthesis of N-(5-15-(1,1-difluoroethyl)benzo[d]oxazol-2-yl)-8-(methylamino 2,7-naphthyridin-3-yl)cyclopropanecarboxamide

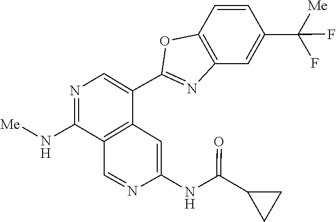 [SMILES: CNc1ncc(-c2nc3cc(C(C)(F)F)ccc3o2)c2cc(NC(=O)C3CC3)ncc12]

### Step 1: 1-(4-(benzyloxy)-3-nitrophenyl)ethan-1-one

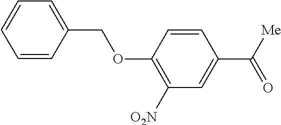 [SMILES: CC(=O)c1ccc(OCc2ccccc2)c([N+](=O)[O-])c1]

To a stirring mixture of 1-(4-hydroxy-3-nitrophenyl)ethan-1-one (2.00 g; 11.04 mmol; 1.00 eq.), K₂CO₃ (4.57 g; 33.067 mmol; 3.00 eq.) in acetone (30 mL) was added (bromomethyl)benzene (2.80 g; 16.371 mmol; 1.48 eq.) was stirred at 60° C. for 4 hours under nitrogen atmosphere. Upon completion, the reaction was concentrated under reduced pressure. The residue was purified via a silica gel column using 0-40% of EtOAc in petroleum ether as eluent to provide 1-(4-(benzyloxy)-3-nitrophenyl)ethan-1-one as a white solid (1.89 g, 61.2%). LCMS (ESI) m/z 272.1, [M+H]⁺.

### Step 2: 1-(benzyloxy)-4-(1,1-difluoroethyl)-2-nitrobenzene

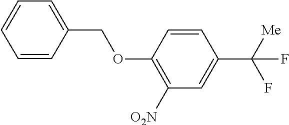 [SMILES: CC(F)(F)c1ccc(OCc2ccccc2)c([N+](=O)[O-])c1]

1-(4-(benzyloxy)-3-nitrophenyl)ethan-1-one (800.0 mg; 2.95 mmol; 1.00 eq.) was added to a solution of DAST (2.85 g; 17.694 mmol; 6.00 eq.) in CH₂Cl₂ at 0° C. under nitrogen atmosphere. The reaction was stirred at 40° C. for 48 hours. The desired product was detected via LCMS. The reaction was quenched by a saturated NaHCO₃ aqueous solution (20 mL). The mixture was extracted by CH₂Cl₂ (3×50 mL). The combined organic phase was dried over anhydrous Na₂SO₄, concentrated under reduce pressure. The residue was purified by flash chromatography on silica gel column using 0-20% of EtOAc in petroleum ether as eluent to provide 1-(benzyloxy)-4-(1,1-difluoroethyl)-2-nitrobenzene as a yellow solid (348.0 mg, 40.2%). LCMS (ESI) m/z 294.1, [M+H]⁺.

### Step 3: 2-amino-4-(1,1-difluoroethyl)phenol

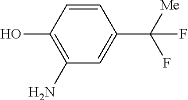 [SMILES: CC(F)(F)c1ccc(O)c(N)c1]

To a stirred mixture of 1-(benzyloxy)-4-(1,1-difluoroethyl)-2-nitrobenzene (100.0 mg; 0.341 mmol; 1.00 eq.) in EtOH (7 mL) was added 20% Pd(OH)₂/C (50.0 mg; 50% w/w) under nitrogen atmosphere. The mixture was hydrogenated at 40 for 2 hours under hydrogen atmosphere (2 atm). After being filtered through a celite pad, the filtrate was concentrated under reduced pressure to afford 2-amino-4-(1,1-difluoroethyl)phenol as a yellow solid (48.0 mg, 75.6%). The crude product was used to the next reaction without further purification. LCMS (ESI) m/z 174.1, [M+H]⁺.

### Step 4: N-(5-(5-(1,1-difluoroethyl)benzo[d]oxazol-2-yl)-8-(methylamino)-2,7-naphthyridin-3-yl)cyclopropanecarboxamide

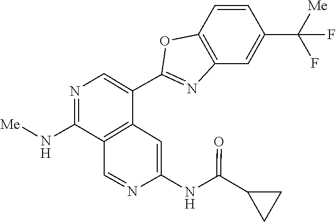 [SMILES: CNc1ncc(-c2nc3cc(C(C)(F)F)ccc3o2)c2cc(NC(=O)C3CC3)ncc12]

N-(5-(5-(1,1-difluoroethyl)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(1,1-difluoroethyl)phenol as the starting material. LCMS (ESI) m/z 424.2, [M+H]⁺. ¹H NMR (400 MHz, DMSO-d₆) δ 11.05 (s, 1H), 9.70 (s, 1H), 9.46 (s, 1H), 8.95 (s, 1H), 8.77-8.67 (m, 1H), 7.92 (d, J=2.0 Hz, 1H), 7.84 (d, J=8.4 Hz, 1H), 7.62-7.56 (m, 1H), 3.10 (d, J=4.4 Hz, 3H), 2.15-2.01 (m, 4H), 0.97-0.82 (m, 4H).

### Example 244: Synthesis of N-(5-(5-(difluoromethyl)benzo[d]oxazol-2-yl)-8-(methylamino)-2,7-naphthyridin-3-yl) cyclopropanecarboxamide

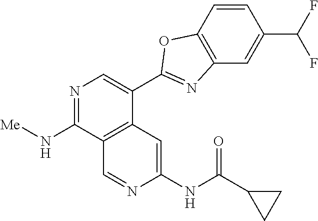 [SMILES: CNc1ncc(-c2nc3cc(C(F)F)ccc3o2)c2cc(NC(=O)C3CC3)ncc12]

### Step 1: 4-(benzyloxy)-3-nitrobenzaldehyde

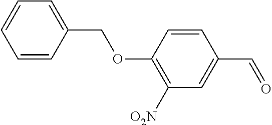 [SMILES: O=Cc1ccc(OCc2ccccc2)c([N+](=O)[O-])c1]

A mixture of 4-hydroxy-3-nitrobenzaldehyde (2.00 g; 11.967 mmol; 1.00 eq.), K₂CO₃ (40.96 g; 35.901 mmol; 3.00 eq.) and (bromomethyl)benzene (3.07 g; 17.951 mmol, 1.5 eq.) in acetone (15 mL) was stirred at 40° C. for 14 hours under nitrogen atmosphere. Upon completion, the resulting mixture was concentrated under reduced pressure. The residue was purified by flash chromatography on silica gel column using 0-30% of EtOAc in petroleum ether as eluent to provide 4-(benzyloxy)-3-nitrobenzaldehyde as a yellow solid (1.00 g, 32.4%). LCMS (ESI) m/z 258.1, [M+H]⁺.

### Step 2: 1-(benzyloxy)-4-(difluoromethyl)-2-nitrobenzene

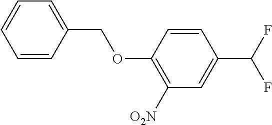 [SMILES: O=[N+]([O-])c1cc(C(F)F)ccc1OCc1ccccc1]

To a stirred solution of 4-(benzyloxy)-3-nitrobenzaldehyde (400.0 mg; 1.555 mmol; 1.00 eq.) in DCM (8 mL) was added DAST (751.9 mg; 4.665 mmol; 3.00 eq.) dropwise at 0° C. under nitrogen atmosphere. The resulting mixture was stirred at room temperature for 14 hours. The resulting mixture was quenched with a saturated sodium bicarbonate solution, extracted with dichloromethane. The combined organic layers were dried over Na₂SO₄, filtered, and concentrated under reduced pressure. The residue was purified by flash chromatography on silica gel column using 0-20% of EtOAc in petroleum ether as eluent to provide 1-(benzyloxy)-4-(difluoromethyl)-2-nitrobenzene as a yellow solid (470.0 mg, 97.4%).

### Step 3: 2-amino-4-(difluoromethyl)phenol

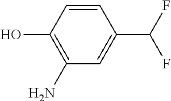 [SMILES: Nc1cc(C(F)F)ccc1O]

To a stirring mixture of 1-(benzyloxy)-4-(difluoromethyl)-2-nitrobenzene (250.0 mg; 0.895 mmol; 1.00 eq.) in EtOH (8 mL) was added 20% Pd(OH)₂/C (50.0 mg; 20% tip-/w) under nitrogen atmosphere. The mixture was hydrogenated at 40° C. for 2 hours under hydrogen atmosphere (2 atm). After being filtered through a celite pad, the filtrate was concentrated under reduced pressure to afford 2-amino-4-(difluoromethyl)phenol as a brown solid (125.0 mg, 82.4%). LCMS (ESI) m/z 160.0, [M+H]⁺.

### Step 4: N-(5-(5-(difluoromethyl)benzo[d]oxazol-2-yl)-8-(methylamino)-2,7-naphthyridin-3-yl)cyclopropanecarboxamide

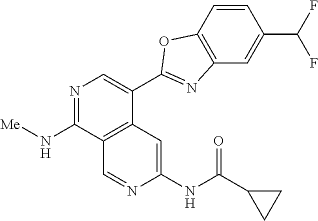 [SMILES: CNc1ncc(-c2nc3cc(C(F)F)ccc3o2)c2cc(NC(=O)C3CC3)ncc12]

N-(5-(5-(difluoromethyl)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(difluoromethyl)phenol as the starting material. LCMS (ESI) m/z 410.1, [M+H]⁺. NMR (400 MHz, DMSO-d₆) δ 11.05 (s, 1H), 9.73 (s, 1H), 9.45 (s, 1H), 8.95 (s, 1H), 8.77-8.66 (m, 1H), 7.97 (d, J=1.6 Hz, 1H), 7.88 (d, J=8.4 Hz, 1H), 7.61 (dd, J=8.4, 1.6 Hz, 1H), 7.19 (t, J=56.0 Hz, 1H), 3.10 (d, J=4.4 Hz, 3H), 2.16-2.05 (m, 1H), 0.97-0.81 (m, 4H).

### Example 245: Synthesis of N-(5-(5-(cyanomethyl)benzo[d]oxazol-2-yl)-8-(methylamino)-2,7-naphthyridin-3-yl)cyclopropanecarboxamide

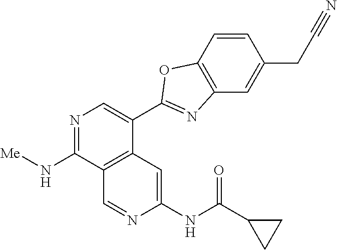 [SMILES: CNc1ncc(-c2nc3cc(CC#N)ccc3o2)c2cc(NC(=O)C3CC3)ncc12]

### Step 1: 2-(4-hydroxy-3-nitrophenyl)acetonitrile

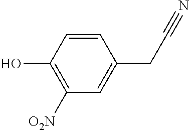 [SMILES: N#CCc1ccc(O)c([N+](=O)[O-])c1]

To a stirring mixture of 2-(4-hydroxyphenyl) acetonitrile (1.00 g; 7.51 mmol: 1.00 eq.) in a mixture solvent of MeCN/water (19:1, 12 mL) was added urea nitrate (924.2 mg; 7.51 mmol; 1.00 eq.). The reaction mixture was stirred at 80° C. for 2 hours. Upon completion, the mixture was concentrated under vacuum. The crude product was purified by flash chromatography on silica gel column using 10-20% of EtOAc in petroleum ether as eluent to provide 2-(4-hydroxy-3-nitrophenyl)acetonitrile as a yellow solid (564.4 mg, 19.2%). LCMS (ESI) m/z 177.0, [M−H]⁻.

### Step 2: 2-(3-amino-4-hydroxyphenyl)acetonitrile

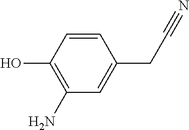 [SMILES: N#CCc1ccc(O)c(N)c1]

To a stirring mixture of 2-(4-hydroxy-3-nitrophenyl)acetonitrile (260.0 mg; 1.459 mmol; 1.00 eq.) in a mixture solvent of MeOH/CH₂Cl₂ (8:1, 22.5 mL) was added 10% Pd/C (77.6 mg; 30% w/w) under nitrogen atmosphere. The mixture was hydrogenated at room temperature for 2 hours under hydrogen atmosphere (2 atm). Upon completion, the resulting mixture was filtered, and concentrated under reduced pressure to provide 2-(3-amino-4-hydroxyphenyl)acetonitrile as a yellow solid (222.1 mg, 44.1%) LCMS (ESI) m/z 149.1, [M+H]⁺.

### Step 3: N-(5-(5-(cyanomethyl)benzo[d]oxazol-2-yl)-8-(methylamino)-2,7-naphthyridin-3-yl)cyclopropanecarboxamide

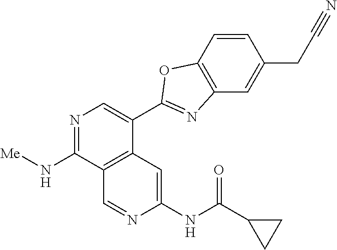 [SMILES: CNc1ncc(-c2nc3cc(CC#N)ccc3o2)c2cc(NC(=O)C3CC3)ncc12]

N-(5-(5-(cyanomethyl)benzo[d]oxazol-2-yl)-8-(methylamino)-2,7-naphthyridin-3-yl)cyclopropanecarboxamide was synthesized using a similar procedure that was previously, described in Example 210, by using N-(5-formyl-8-(methylamino)-2,7-naphthyridin-3-yl)cyclopropanecarboxamide and 2-(3-amino-4-hydroxyphenyl)acetonitrile as the starting material. LCMS (ESI) m/z 399.1, [M+H]⁺. ¹H NMR (400 MHz, DMSO-d₆) δ 11.04 (s, 1H), 9.74 (s, 1H), 9.44 (s, 1H), 8.93 (s, 1H), 8.71-8.65 (m, 1H), 7.80-7.70 (m, 2H), 7.37 (dd, J=8.4, 1.6 Hz, 1H), 4.18 (s, 2H), 3.09 (d, =4.4 Hz, 3H), 2.14-2.07 (m, 1H), 0.95-0.81 (m, 4H).

### Example 246: Synthesis of N-(5-(5-ethoxybenzo[d]oxazol-2-yl)-8-(methylamino)-2,7-naphthyridin-3-yl)cyclopropanecarboxamide

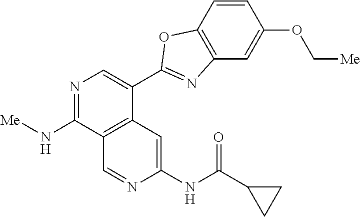 [SMILES: CCOc1ccc2oc(-c3cnc(NC)c4cnc(NC(=O)C5CC5)cc34)nc2c1]

### Step 1: 4-ethoxy-2-nitrophenol

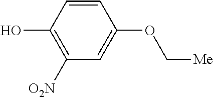 [SMILES: CCOc1ccc(O)c([N+](=O)[O-])c1]

To a stirring mixture of 4-ethoxyphenol (1.00 g; 3.619 mmol; 1.00 eq.) in a mixture solvent of MeCN/water (20:1, 10 mL) was added urea nitrate (445.1 mg; 3.619 mmol, 1.00 eq.) at 0° C. under nitrogen atmosphere. The reaction was stirred at 80° C. for 1 hour. Upon completion, the resulting mixture was concentrated under reduced pressure. The residue was purified by flash chromatography on silica gel column using 10-60% of EtOAc in petroleum ether as eluent to provide 4-ethoxy-2-nitrophenol as white oil (618.0 mg, 46.6%). LCMS (ESI) m/z 182.1, [M+H]⁺.

### Step 2: 2-amino-4-ethoxyphenol

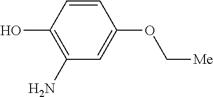 [SMILES: CCOc1ccc(O)c(N)c1]

To a stirred mixture of 4-ethoxy-2-nitrophenol (300.0 mg; 1.638 mmol; 1.00 eq.) in MeOH (25 mL) was added 10% Pd/C (300.0 mg; 100% w/w) under nitrogen atmosphere. The mixture was hydrogenated at 40° C. for 3 hours under hydrogen atmosphere (2 atm). Upon completion, the reaction mixture was filtered. The filtrate was concentrated under reduced pressure. The residue was purified by flash chromatography on pre-packed C18 column using 20-60% of ACN in water (10 mmol/L, NH₄HCO₃) as eluent to provide 2-amino-4-ethoxyphenol as white solid (260.0 mg, 90.1%). LCMS (ESI) m/z 154.1, [M+H]⁺.

### Step 3: N-(5-(5-ethoxybenzo[d]oxazol-2-yl-8-ethylamino)-2,7-naphthyridin-3-yl)cyclopropanecarboxamide

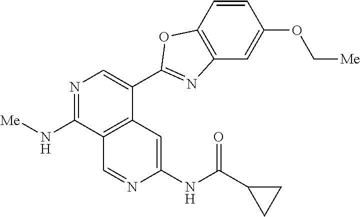 [SMILES: CCOc1ccc2oc(-c3cnc(NC)c4cnc(NC(=O)C5CC5)cc34)nc2c1]

N-(5-(5-ethoxybenzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-ethoxyphenol as the starting material. LCMS (ESI) m/z 404.2, [M+H]⁺. ¹H NMR (400 MHz, DMSO-d₆) δ 11.04 (s, 1H), 9.64 (s, 1H), 9.44 (s, 1H), 8.89 (s, 1H), 8.68-8.60 (m, 1H), 7.61 (d, J===8.8 Hz, 1H), 7.26 (d, J=2.4 Hz, 6.95 (dd, J=8.8, 2.4 Hz, 1H) 4.17-4.07 (m, 2H), 3.09 (d, J=4.4 Hz, 3H), 2.15-2.06 (m, 1H), 1.38 (t, J=7.2 Hz, 3H), 0.96-0.82 (m, 4H).

### Example 247: Synthesis of N-(5-(5-(2,2-difluoroethoxy)benzo[d]oxazol-2-yl)-8-(methylamino)-2,7-naphthyridin-3yl)cyclopropanecarboxamide

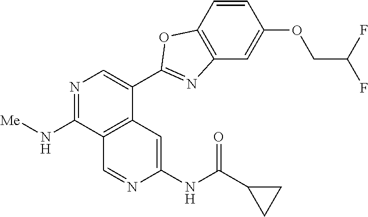 [SMILES: CNc1ncc(-c2nc3cc(OCC(F)F)ccc3o2)c2cc(NC(=O)C3CC3)ncc12]

### Step 1: 1-(benzyloxy)-4-(2,2-difluoroethoxy)benzene

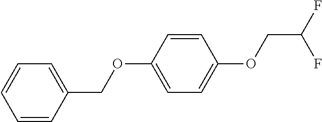 [SMILES: FC(F)COc1ccc(OCc2ccccc2)cc1]

To a stirred mixture 4-(benzyloxy)phenol (2.00 g; 9.981 mmol; 1.00 eq.) and Cs₂CO₃ (6.52 g; 20.001 mmol; 2.00 eq.) in DMF (20 mL) was added 2,2-difluoroethyl trifluoromethanesulfonate (2.57 g; 12.080 mmol; 1.20 eq.). The mixture was stirred at 60° C. for 1 hour under nitrogen atmosphere. The reaction mixture was cooled to RT and was diluted with water (100 mL) and precipitated product were collected by filtration and washed with water (50 mL) to afford 1-(benzyloxy)-4-(2,2-difluoroethoxy)benzene as a white solid (2.50 g, 94.7%). LCMS (ESI) m/z 265.1, [M+H]⁺.

### Step 2: 4-(2,2-difluoroethoxy)phenol

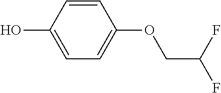 [SMILES: Oc1ccc(OCC(F)F)cc1]

To a stirring mixture of 1-(benzyloxy)-4-(2,2-difluoroethoxy)benzene (2.50 g; 9.462 mmol; 1.00 eq.) in MeOH (25 mL) was added 10% Pd/C (750.3 mg; 30% w/w) under nitrogen atmosphere. The mixture was hydrogenated at 40° C. for 1 hour under hydrogen atmosphere (2 atm). Upon completion, the resulting mixture was filtered. The filter cake was washed with MeOH. The filtrate was concentrated under reduced pressure to afford 4-(2,2-difluoroethoxy)phenol as a white solid (1.65 g, 99.6%). LCMS (ESI) m/z 175.0, [M+H]⁺.

### Step 3: 4-(2,2-difluoroethoxy)-2-nitrophenol

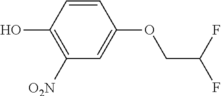 [SMILES: O=[N+]([O-])c1cc(OCC(F)F)ccc1O]

A mixture of urea nitrate (1.16 g; 9.426 mmol; 1.00 eq.) and 4-(2,2-difluoroethoxy)phenol (1.65 g; 9.474 mmol; 1.00 eq.) in a mixture solvent of MeCN/water (19:1, 15 mL) was stirred at 80° C. for 1 hour under nitrogen atmosphere. The desired product was detected via LCMS. The resulting mixture was diluted with water (100 mL) and extracted with EtOAc (3×50 mL). The combined organic layers were washed with brine (3×50 mL), dried over anhydrous Na₂SO₄, filtered, and concentrated under reduced pressure. The residue was purified by flash chromatography on silica gel column using 10-30% of EtOAc in petroleum ether as eluent to provide 4-(2,2-difluoroethoxy)-2-nitrophenol as a yellow solid (1.01 g, 48.4%). LCMS (ESI) m/z 218.0, [M−H]⁻.

### Step 4: 2-amino-4-(2,2-difluoroethoxy)phenol

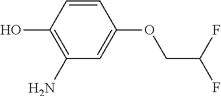 [SMILES: Nc1cc(OCC(F)F)ccc1O]

To a stirred mixture of 4-(2,2-difluoroethoxy)-2-nitrophenol (500.1 mg; 2,281 mmol; 1.00 eq.) in MeOH (5 mL) was added 10% Pd/C (150.2 mg; 30% w/w) under nitrogen atmosphere. The mixture was hydrogenated at 40° C. for 1 hour under hydrogen atmosphere (2 atm). The resulting mixture was filtered, the filter cake was washed with MeOH. The filtrate was concentrated under reduced pressure to afford 2-amino-4-(2,2-difluoroethoxy)phenol as a brown oil (400.2 mg, 92.6%). LCMS (ESI) m/z 190.1, [M+H]⁺.

### Step 5: N-(5-(5-(2,2-difluoroethoxy)benzo[d]oxazol-2-yl)-8-(methylamino)-2,7-naphthyridin-3-yl)cyclopropanecarboxamide

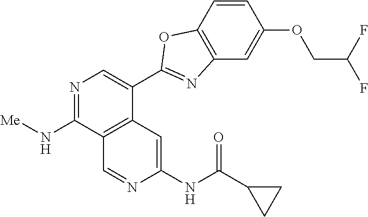 [SMILES: CNc1ncc(-c2nc3cc(OCC(F)F)ccc3o2)c2cc(NC(=O)C3CC3)ncc12]

N-(5-(5-(2,2-difluoroethoxy)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(2,2-difluoroethoxy)phenol as the starting material. LCMS (ESI) m/z 440.1, [M+H]⁺. ¹H NMR (400 MHz, DMSO-d₆) δ 11.04 (s, 1H), 9.65 (s, 1H), 9.43 (s, 1H), 8.90 (s, 1H), 8.69-8.61 (m, 1H), 7.65 (d, J=8.8 Hz, 1H), 7.40 (d, J=2.4 Hz, 1H), 7.03 (dd, J=8.8, 2.4 Hz, 1H), 6.60-6.27 (m, 1H), 4.50-4.36 (m, 2H), 3.09 (d, J=4.4 Hz, 3H), 2.16-2.06 (m, 1H), 0.96-0.82 (m, 4H).

### Example 248: Synthesis of N-(5-(5-(methoxymethyl)benzo[d]oxazol-2-yl)-8-(methylamino 2,7-naphthyridin-3-yl)cyclopropanecarboxamide

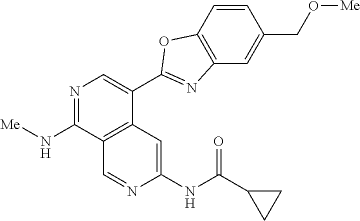 [SMILES: CNc1ncc(-c2nc3cc(COC)ccc3o2)c2cc(NC(=O)C3CC3)ncc12]

### Step 1: 4-(methoxymethyl)-2-nitrophenol

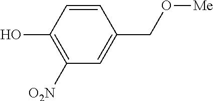 [SMILES: COCc1ccc(O)c([N+](=O)[O-])c1]

A mixture of 4-(methoxymethyl)phenol (200.1 mg; 1.442 mmol; 1.00 eq.) and HNO₃ (30% in water, 5 mL) in MeCN (10 mL) was stirred at room temperature for 1 hour under nitrogen atmosphere. The desired product was detected via LCMS. The reaction was diluted with water (2 mL) and extracted with EtOAc (2×20 mL). The combined organic layers were washed with brine (2×20 mL), dried over anhydrous Na₂SO₄, filtered, and concentrated under reduced pressure. The residue was purified by flash chromatography on silica gel column using 5-10% of EtOAc in petroleum ether as eluent to provide 4-(methoxymethyl)-2-nitrophenol as a yellow oil (140.2 mg, 52.8%). LCMS (ESI) m/z 182.1, [M+H]⁺.

### Step 2: 2-amino-4 (methoxymethyl)phenol

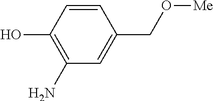 [SMILES: COCc1ccc(O)c(N)c1]

To a stirring mixture of 4-(methoxymethyl)-2-nitrophenol (140.2 mg; 0.764 mmol; 1.00 eq.) in MeOH (3 mL) was added 10% Pd/C (42.2 mg; 30% w/w) under nitrogen atmosphere. The mixture was hydrogenated at 40 for 1 hour under hydrogen atmosphere (2 atm). The desired product was detected via LCMS. The resulting mixture was filtered, the filter cake was washed with MeOH. The filtrate was concentrated under reduced pressure to afford 2-amino-4-(methoxymethyl)phenol as a white solid (70.2 mg, 59.7%). LCMS (ESI) m/z 154.1, [M+H]⁺.

### Step 3: N-(5-(5-(methoxymethyl)benzo[d]oxazol-2-yl)-8-(methylamino)-2,7-naphthyridin-3-yl)cyclopropanecarboxamide

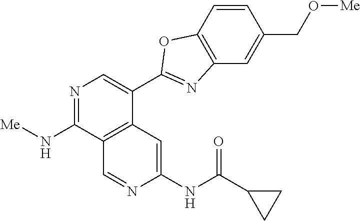 [SMILES: CNc1ncc(-c2nc3cc(COC)ccc3o2)c2cc(NC(=O)C3CC3)ncc12]

N-(5-(5-(methoxymethyl)benzo[d]oxazol-2-yl)-8-(methylamino)-2,7-naphthyridin-3-yl)cyclopropanecarboxamide was synthesized using a similar procedure that was previously, described in Example 210, by using N-(5-formyl-8-(methylamino)-2,7-naphthyridin-3-yl)cyclopropanecarboxamide and 2-amino-4-(methoxymethyl)phenol as the starting material. LCMS (ESI) m/z 404.2, [M+H]⁺. ¹H NMR (400 MHz, DMSO-d₆) δ 11.04 (s, 1H), 9.73 (s, 1H), 9.44 (s, 1H), 8.92 (s, 1H), 8.69-8.61 (m, 11-11), 7.74-7.67 (m, 2H), 7.37-7.32 (m, 1H), 4.56 (s, 2H), 3.33 (s, 3H), 3.10 (d, J=4.4 Hz, 3H), 2.15-2.06 (m, 1H), 0.96-0.82 (m, 4H).

### Example 249: Synthesis of N-(8-(methylamino)-5-(5-(3-methyltetrahydro-2/1-pyran-4-yl)benzo[d]oxazol-2-yl)-2,7-naphthyridin-3-yl)cyclopropanecarboxamide

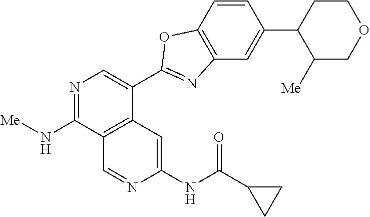 [SMILES: CNc1ncc(-c2nc3cc(C4CCOCC4C)ccc3o2)c2cc(NC(=O)C3CC3)ncc12]

### Step 1: 4-(4-(benzyloxy)phenyl)-3,6-dihydro-2H-pyran

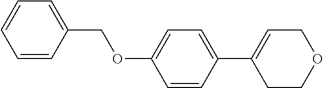 [SMILES: C1=C(c2ccc(OCc3ccccc3)cc2)CCOC1]

To a stirring mixture of 1-(benzyloxy)-4-bromobenzene (2.00 g; 7.600 mmol; 1.00 eq.) in a mixture solvent of 1,4-dioxane/water (5:1, 24 mL) were added XPhos (362.3 mg; 0.760 mmol; 0.10 eq.), XPhos Pd G₃ (643.3 mg; 0.760 mmol; 0.10 eq.), K₃PO₄ (4.84 g; 22.800 mmol; 3.00 eq.) and 2-(3,6-dihydro-2H-pyran-4-yl)-4,4,5,5-tetramethyl-1,3,2-dioxaborolane (1.59 g; 7.600 mmol; 1.00 eq.) under nitrogen atmosphere. The reaction was stirred at 90° C. for 1 hour. The desired product was detected via LCMS. The resulting mixture was cooled and concentrated under reduced pressure. The crude residue was purified by flash chromatography on silica gel column using 10-30% of EtOAc in petroleum ether as eluent to provide 4-(4-(benzyloxy) phenyl)-3,6-dihydro-2H-pyran as white oil (1.36 g, 67.5%). LCMS (ESI) m/z 267.1, [M+H]⁺.

### Step 2: 6-(4-(benzyloxy)phenyl)-3-oxabicyclo[4.1.0]heptane

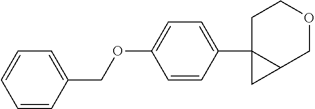 [SMILES: c1ccc(COc2ccc(C34CCOCC3C4)cc2)cc1]

A solution of diethylzinc (1 M in hexane, 9.7 mL) and diiodomethane (2.61 g; 9.762 mmol; 2.00 eq.) in CH₂Cl₂ (20 mL) was stirred for 10 mins at 0° C. under nitrogen atmosphere. To the above solution were added 4-(4-(benzyloxy) phenyl)-3,6-dihydro-2H-pyran (1.30 g; 4.881 mmol; 1.00 eq.) and trifluoroacetic acid (80 mg; 0.732 mmol; 0.15 eq.). The mixture was stirred at room temperature for 3 hours. The resulting mixture was diluted with CH₂HCl₂ (20 mL) and washed with brine (3×5 mL). The organic phase was dried over anhydrous sodium sulfate, filtered, and concentrated under reduced pressure. The residue was purified by flash chromatography on silica gel column using 0-20% of MeOH in CH₂Cl₂ as eluent to provide 6-(4-(benzyloxy)phenyl)-3-oxabicyclo[4.1.0]heptane as a yellow oil (700 mg, 51.1%).

### Step 3: 4-(3-methyltetrahydro-2H-pyran-4-yl)phenol

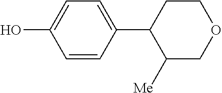 [SMILES: CC1COCCC1c1ccc(O)cc1]

To a solution of 6-(4-(benzyloxy)phenyl)-3-oxabicyclo[4.1.0]heptane (400.0 mg; 1,427 mmol; 1.00 eq.) in MeOH (2.0 mL) was added 10% Pd/C (400.0 mg; 100% w/w) under nitrogen atmosphere. The mixture was hydrogenated at 40° C. for 3 hours under hydrogen atmosphere (2 atm). LC-MS indicated that the starting material was consumed. After filtration, the filtrate was concentrated under reduced pressure to afford 4-(3-methyltetrahydro-2H-pyran-4-yl)phenol as a colorless oil (271.0 mg, crude). LCMS (ESI) m/z 191.1, [M+H]⁺.

### Step 4: 4-(3-methyltetrahydro-2H-pyran-4-yl)-2-nitrophenol

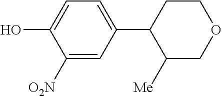 [SMILES: CC1COCCC1c1ccc(O)c([N+](=O)[O-])c1]

To a solution of 4-(3-methyltetrahydro-2H-pyran-4-yl)phenol (271.0 mg; 1.306 mmol; 1.00 eq.) in a mixture solvent of MeCN/water (20:1, 10 mL) was added urea nitrate (160.6 mg; 1.306 mmol; 1.00 eq.) at 0° C. under nitrogen atmosphere. The reaction was stirred at 80° C. for 1 hour. The desired product was detected via LCMS. The resulting mixture was concentrated under reduced pressure. The residue was purified by flash chromatography on silica gel column using 40-60% of EtOAc in petroleum ether as eluent to provide 4-(3-methyltetrahydro-2H-pyran-4-yl)-2-nitrophenol as colorless oil (252.0 mg, 81.4%). LCMS (ESI) m/z 236.1, [M−H]⁻.

### Step 5: 2-amino-4-(3-methyltetrahydro-2H-pyran-4-yl)phenol

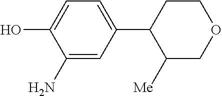 [SMILES: CC1COCCC1c1ccc(O)c(N)c1]

To a solution of 4-(3-methyltetrahydro-2H-pyran-4-yl)-2-nitrophenol (150.0 mg; 0.638 mmol; 1.00 eq.) in MeOH (10 mL) was added 10% Pd/C (150.0 mg; 100% w/w) under nitrogen atmosphere. The mixture was hydrogenated at 40° C. for 3 hours under hydrogen atmosphere (2 atm). The desired product was detected via LCMS. After filtration, the filtrate was concentrated under reduced pressure to provide 2-amino-4-(3-methyltetrahydro-2H-pyran-4-yl)phenol as colorless oil (129.0 mg, crude). LCMS (ESI) m/z 208.1, [M+H]⁺.

### Step 6: N-(8-(methylamino)-5-(5-(3-methyltetrahydro-2-pyran-4-yl)benzo[d]oxazol-2,7-naphthyridin-3-yl)cyclopropanecarboxamide

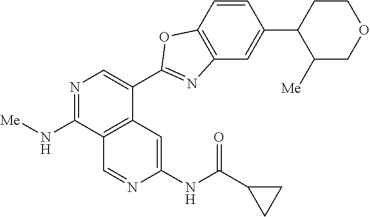 [SMILES: CNc1ncc(-c2nc3cc(C4CCOCC4C)ccc3o2)c2cc(NC(=O)C3CC3)ncc12]

N-(8-(methylamino)-5-(5-(3-m ethyltetrahydro-2H-pyran-4-yl)benzo[d]oxazol-2-yl)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(3-methyltetrahydro-2H-pyran-4-yl)phenol as the starting material. LCMS (ESI) m/z 458.2, [M+H]⁺. ¹H NMR (400 MHz, DMSO-d₆) δ 11.04 (s, 1H), 9.69 (s, 1H), 9.44 (s, 1H), 8.90 (s, 1H), 8.68-8.60 (m, 1H), 7.64 (d, J=8.4 Hz, 7.63 (d, J=1.2 Hz, 1H), 7.25 (dd, J=8.4, 1.2 Hz, 1H), 4.02-3.83 (m, 2H), 3.50-3.39 (m, 1H), 3.09 (d, J=4.4 Hz, 3H), 3.07-3.00 (m, 1H), 2.50-2.45 (m, 1H), 2.16-2.06 (m, 1H), 1.95-1.77 (m, 2H), 1.72-1.63 (m, 1H), 0.97-0.82 (m, 4H), 0.57 (d, J=6.8 Hz, 3H).

### Example 250: Synthesis of N-(8-(methylamino)-5-(5-((1-(methylsulfonyl)azetidin-3-yl)oxy)benzo[d]oxazol-2-yl)-2,7-naphthyridin-3-yl)cyclopropanecarboxamide

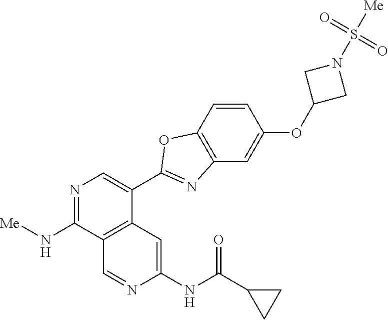 [SMILES: CNc1ncc(-c2nc3cc(OC4CN(S(C)(=O)=O)C4)ccc3o2)c2cc(NC(=O)C3CC3)ncc12]

### Step 1: 3-(4-(benzyloxy)phenoxy)-1-(methylsulfonyl)azetidine

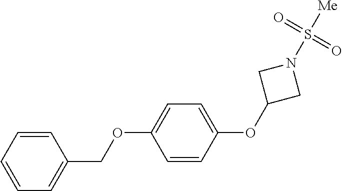 [SMILES: CS(=O)(=O)N1CC(Oc2ccc(OCc3ccccc3)cc2)C1]

To a solution of 3-(4-(benzyloxy)phenoxy)azetidine hydrochloride (Example 230, step 2) (500.0 mg; 1.714 mmol; 1.00 eq.) and DIPEA (664.4 mg; 5.142 mmol; 3.00 eq.) in dichloromethane (5 mL) was added methanesulfonic anhydride (447.7 mg; 2.571 mmol; 1.50 eq.) at 0° C. The resulting solution was stirred at room temperature for 1 hour. The desired product was detected via LCMS. The mixture was diluted with EtOAc (100 mL) and washed with brine (3×5 mL). The organic layer was dried with Na₂SO₄, filtered and the solvent was removed under reduced pressure. The residue was purified by flash chromatography on silica gel column using 1-3% of MeOH CH₂Cl₂ as eluent to provide 3-(4-(benzyloxy)phenoxy)-1-(methylsulfonyl)azetidine as a white solid (420.0 mg, 73.5%). LCMS (ESI) m/z 334.1, [M+H]⁺.

### Step 2: 4-((1-(methylsulfonyl)azetidin-3-yl)oxy)phenol

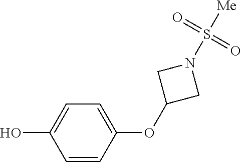 [SMILES: CS(=O)(=O)N1CC(Oc2ccc(O)cc2)C1]

To a stirring solution of 3-(4-(benzyloxy)phenoxy)-1-(methylsulfonyl)azetidine (420.0 mg; 1.260 mmol; 1.00 eq.) in MeOH (10 mL) was added 10% Pd/C (84.0 mg; 20% w/w) under nitrogen atmosphere. The mixture was hydrogenated at room temperature for 1 hour under hydrogen atmosphere (2 atm). The desired product was detected via LCMS. The resulting mixture was filtered, the filter cake was washed with MeOH. The filtrate was concentrated under a reduced pressure to afford 4-((1-(methylsulfonyl)azetidin-3-yl)oxy)phenol as an off-white solid (250.0 mg, 81.6%). LCMS (ESI) m/z 244.1, [M+H]⁺.

### Step 3: 4-#1-(methylsulfonyl)azetidin-3-yl)oxy)-2-nitrophenol

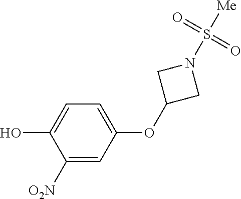 [SMILES: CS(=O)(=O)N1CC(Oc2ccc(O)c([N+](=O)[O-])c2)C1]

A mixture of 4-((1-(methylsulfonyl)azetidin-3-yl)oxy)phenol (250.0 mg; 1.028 mmol; 2.00 eq.) and urea nitrate (61.7 mg; 1.028 mmol; 1.00 eq.) in a mixture solvent of MeCN/water (19:1, 3 mL) was stirred at 80° C. for 1 hour under nitrogen atmosphere. Upon completion, the mixture was allowed to cool down to room temperature. The resulting mixture was concentrated under reduced pressure. The residue was purified by flash chromatography on silica gel column using 3-5% of MeOH in CH₂Cl₂ as eluent to provide 4-((1-(methylsulfonyl)azetidin-3-yl)oxy)-2-nitrophenol as an off-white solid (120.0 mg, 40.5%). LCMS (ESI) m/z 287.0, [M−H]⁻.

### Step 4: 2-amino-4-((1-(methylsulfonyl)azetidin-3-yl)oxy)phenol

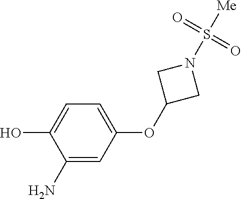 [SMILES: CS(=O)(=O)N1CC(Oc2ccc(O)c(N)c2)C1]

To a stirring mixture of 4((1-(methylsulfonyl)azetidin-3-yl)oxy)-2-nitrophenol (120.0 mg; 0.416 mmol; 1.00 eq.) in MeOH (5 mL) was added 10% Pd/C (42.1 mg; 35% w/w) under nitrogen atmosphere. The mixture was hydrogenated at room temperature for 1 hour under hydrogen atmosphere (2 atm). The desired product was detected via LCMS. The resulting mixture was filtered, the filter cake was washed with MeOH. The filtrate was concentrated under reduced pressure to afford 2-amino-4-((1-(methylsulfonyl)azetidin-3-yl)oxy)phenol as a light yellow solid (1000) mg, 93.2%). LCMS (ESI) m/z 259.1, [M+H]⁺.

### Step 5: N-(8-(methylamino)-5-(5-((1-(methylsulfonyl)azetidin-3-yl)oxy)benzo[d]oxazol-2-2,7-naphthyridin-3-yl)cyclopropanecarboxamide

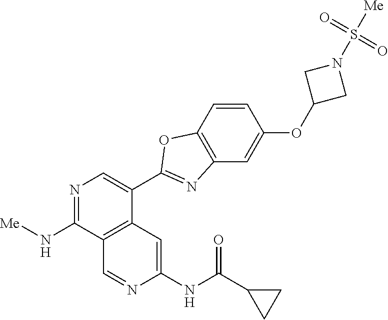 [SMILES: CNc1ncc(-c2nc3cc(OC4CN(S(C)(=O)=O)C4)ccc3o2)c2cc(NC(=O)C3CC3)ncc12]

N-(8-(methylamino)-5-(5-((1-(methylsulfonyl)azetidin-3-yl)oxy)benzo[d]oxazol-2-yl)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-((1-(methylsulfonyl)azetidin-3-yl)oxy)phenol as the starting material. LCMS (ESI) m/z 509.2, [M+H]⁺. ¹H NMR (300 MHz, DMSO-d₆) δ 11.02 (s, 1H), 9.57 (s, 1H), 9.44 (s, 1H), 8.90 (s, 1H), 8.69-8.61 (m, 1H), 7.66 (d, J=8.8 Hz, 1H), 7.21 (d, J=2.4 Hz, 1H), 6.96 (dd, J=8.8, 2.4 Hz, 1H), 5.22-5.13 (m, 1H), 4.42-4.32 (m, 2H), 4.01-3.92 (m, 2H), 3.10 (d, J=4.4 Hz, 3H), 3.09 (s, 3H), 2.14-2.06 (m, 1H), 0.94-0.83 (m, 4H).

### Example 251: Synthesis of N-(8-(methylamino)-5-(5-((1-(tetrahydrofuran-3-yl)azetidin-3-yl)oxy)benzo[d]oxazol-2-yl)-2,7-naphthyridin-3-yl)cyclopropanecarboxamide

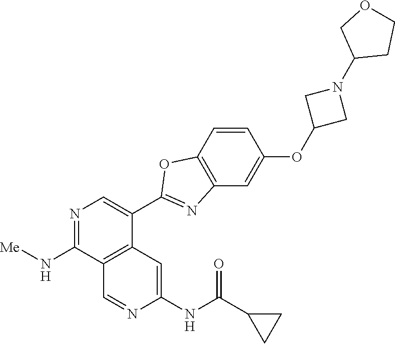 [SMILES: CNc1ncc(-c2nc3cc(OC4CN(C5CCOC5)C4)ccc3o2)c2cc(NC(=O)C3CC3)ncc12]

### Step 1: 3-(4-(benzyloxy)phenoxy)-1-(tetrahydrofuran-3-yl)azetidine

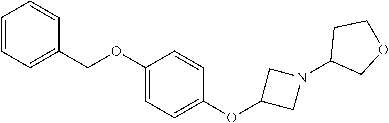 [SMILES: c1ccc(COc2ccc(OC3CN(C4CCOC4)C3)cc2)cc1]

To a mixture of 3-(4-(benzyloxy)phenoxy)azetidine hydrochloride (Example 230, step 2) (2.00 g; 6,854 mmol; 1.00 eq.) and di hydrofuran-3(2H)-one (590.0 mg; 6.854 mmol; 1.00 eq.) in CH₂Cl₂ (120 mL) was added NaBH(OAc); (2.91 g; 13.708 mmol; 2.00 eq.) in portions. The resulting solution was stirred at room temperature for 2 hours under nitrogen atmosphere. The resulting mixture was diluted with CH₂Cl₂ (200 mL) and washed with a saturated sodium bicarbonate solution (3×40 mL). The organic layer was dried over anhydrous sodium sulfate, filtered, and concentrated under vacuum. The residue was purified by flash chromatography on silica gel column using 2-10% of MeOH in CH₂Cl₂ as eluent to provide 3-(4-(benzyloxy)phenoxy)-1-(tetrahydrofuran-3-yl)azetidine as a brown solid (1.36 g, 61%). LCMS (ESI) m/z 326.2, [M+H]⁺.

### Step 2: 4-((1-(tetrahydrofuran-3-yl)azetidin-3-yl)oxy)phenol

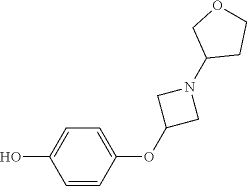 [SMILES: Oc1ccc(OC2CN(C3CCOC3)C2)cc1]

To a stirring mixture of 3-(4-(benzyloxy)phenoxy)-1-(tetrahydrofuran-3-yl)azetidine (1.36 g; 3,995 mmol; 1.00 eq.) in MeOH (10 mL) was added 10% Pd/C (1.36 g; 100% w/w) under nitrogen atmosphere. The mixture was hydrogenated at room temperature for 3 hours under hydrogen atmosphere (2 atm), After filtration, the filtrate was concentrated under reduced pressure to afford 4-((1-(tetrahydrofuran-3-yl)azetidin-3-yl)oxy)phenol as a brown solid (977 mg, crude). LCMS (ESI) m/z 236.1, [M+H]⁺.

### Step 3: 2-nitro-4-((1-(tetrahydrofuran-3-yl)azetidin-3-yl)oxy)phenol

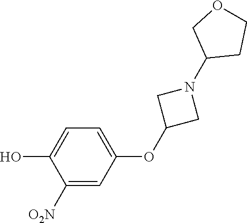 [SMILES: O=[N+]([O-])c1cc(OC2CN(C3CCOC3)C2)ccc1O]

To a stirring mixture of 4-((1-(tetrahydrofuran-3-yl)azetidin-3-yl)oxy)phenol (377.0 mg; 1.941 mmol 1.00 eq.) in a mixture of MeCN/water (95:5, 10 mL) was added urea nitrate (238.8 mg; 1.941 mmol; 1.00 eq.) at 0° C. under nitrogen atmosphere. The reaction was stirred at 80° C. for 1 hour. The resulting mixture was cooled and concentrated under reduced pressure. The residue was purified by flash chromatography on silica gel column using 0-10% of MeOH in CH₂Cl₂ as eluent to provide 2-nitro-4-((1-(tetrahydrofuran-3-yl)azetidin-3-yl)oxy)phenol as a red solid (230.0 mg, 50.0%). LCMS (ESI) m/z 281.1, [M+H]⁺.

### Step 4: 2-amino-4-((1-(tetrahydrofuran-3-yl)azetidin-3-yl)oxy)phenol

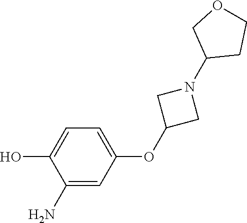 [SMILES: Nc1cc(OC2CN(C3CCOC3)C2)ccc1O]

To a solution of 2-nitro-44(1-(tetrahydrofuran-3-yl)azetidin-3-yl)oxy)phenol (160.0 mg; 0.571 mmol; 1.00 eq.) in a mixture solvent of MeOH/CH₂Cl₂(4:1, 16 mL) was added 10% Pd/C (160.0 mg; 100% w/w) under nitrogen atmosphere. The mixture was hydrogenated at 40 cc for 3 hours under hydrogen atmosphere (2 atm). The desired product was detected via LCMS. After filtration, the filtrate was concentrated under reduced pressure to provide 2-amino-4-((1-(tetrahydrofuran-3-yl)azetidin-3-yl)oxy)phenol as white oil (92.0 mg, crude). The crude product was taken directly to the next reaction without further purification. LCMS (ESI) m/z 251.1, [M+H]⁺.

### Step 5: N-(8-(methylamino)-5-(5-((1-(tetrahydrofuran-3-yl)azetidin-3-yl)oxy)benzo[d]oxazol-2-yl)-2,7-naphthyridin-3-yl)cyclopropanecarboxamide

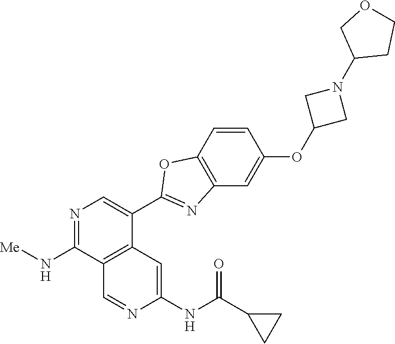 [SMILES: CNc1ncc(-c2nc3cc(OC4CN(C5CCOC5)C4)ccc3o2)c2cc(NC(=O)C3CC3)ncc12]

N-(8-(methylamino)-5-(5-((1-(tetrahydrofuran-3-yl)azetidin-3-yl)oxy)benzo[d]oxazol-2-yl)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-((1-(tetrahydrofuran-3-yl)azetidin-3-yl)oxy)phenol as the starting material. LCMS (ESI) m/z 501.2, [M+H]⁺. ¹H NMR (400 MHz, DMSO-d₆) 311.01 (s, 1H), 9.58 (s, 1H), 9.44 (s, 1H), 8.89 (s, 1H), 8.67-8.59 (m, 1H), 7.63 (d, J=8.8 Hz, 1H), 7.12 (d, J=2.4 Hz, 1H), 6.91 (dd, J=8.8, 2.4 Hz, 1H), 4.95-4.85 (m, 1H), 3.81-3.70 (m, 3H), 3.70-3.64 (m, 1H), 3.62-3.54 (m, 1H), 3.51-3.43 (m, 1H), 3.09 (d, J=4.4 Hz, 3H), 3.08-3.01 (m, 3H), 2.16-2.05 (m, 1H), 1.88-1.75 (m, 1H), 1.68-1.63 (m, 1H), 0.96-0.82 (m, 4H).

### Example 252: Synthesis of N-(8-(methylamino)-5-(5-(1-(methylsulfonyl)azetidin-3-yl)benzo[d]oxazol-2-yl)-2,7-naphthyridin-3-yl)cyclopropanecarboxamide

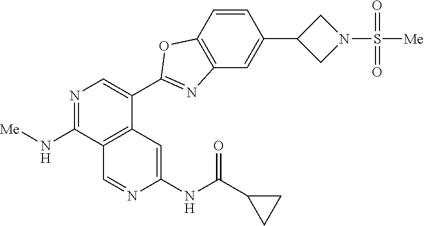 [SMILES: CNc1ncc(-c2nc3cc(C4CN(S(C)(=O)=O)C4)ccc3o2)c2cc(NC(=O)C3CC3)ncc12]

Seep 1: tert-butyl 3-(4-(benzyloxy)phenyl)azetidine-1-carboxylate

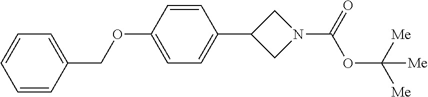 [SMILES: CC(C)(C)OC(=O)N1CC(c2ccc(OCc3ccccc3)cc2)C1]

To a solution of (4-(benzyloxy)phenyl)boronic acid (1.61 g; 7.064 mmol; 2.00 eq.) in i-PrOH (15 mL) was added nickel(II) iodide (CAS: 13462-90-3) (551.9 mg; 1.766 mmol, 0.50 eq.) and (1R,2R)-2-aminocyclohexan-1-ol (203.4 mg; 1.766 mmol, 0.50 eq.). To the above solution was added drop-wise a solution of NaHMDS in THF (2 M in THF, 3.5 mL) and tert-butyl 3-iodoazetidine-1-carboxylate (1.00 g; 3.532 mmol; 1.00 eq.) at room temperature. The resulting solution was stirred at 80° C. for 3 hours under nitrogen atmosphere. Upon completion, the resulting mixture was concentrated under vacuum. The residue was diluted with water (100 mL) and extracted with CH₂Cl₂. (3×100 MLA The combined organic layers were dried over anhydrous Na₂SO₄, filtered, and concentrated under reduced pressure. The residue was purified by flash chromatography on silica gel column using 30-70% of EtOAc in petroleum ether as eluent to provide tort-butyl 3-(4-(benzyloxy)phenyl)azetidine-1-carboxylate as a white solid (550.0 mg, 45.8%). LCMS (ESI) m/z 338.2, [M−H]⁻.

### Step 2: 3-(4-(benzyloxy)phenyl)azetidine

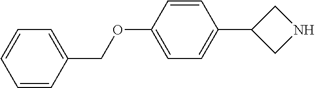 [SMILES: c1ccc(COc2ccc(C3CNC3)cc2)cc1]

To a solution of tert-butyl 3-(4-(benzyloxy)phenyl)azetidine-1-carboxylate (350.0 mg; 1.031 mmol; 1.00 eq.) in CH₂Cl₂ (10 mL) was added TEA (2 mL). The mixture was stirred at room temperature for 2 hours. LCMS indicated that the reaction was complete. The resulting mixture was concentrated under reduced pressure. The residue was purified by flash chromatography on pre-packed C18 column using 10-50% of MeCN in water (10 mmol/L NH₄HCO₃) as eluent to provide 3-(4-(benzyloxy)phenyl)azetidine as a white solid (180.0 mg, 72.9%). LCMS (ESI) m/z 240.1, [M+H]⁺.

### Step 3: 3-(4-(benzyloxy)phenyl)-1-(methylsulfonyl)azetidine

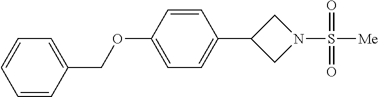 [SMILES: CS(=O)(=O)N1CC(c2ccc(OCc3ccccc3)cc2)C1]

To a solution of 3-(4-(benzyloxy)phenyl)azetidine (180.0 mg; 0.753 mmol; 1.00 eq.) and DIPEA (291.4 mg; 2.259 mmol; 3.00 eq.) in CH₂Cl₂ (10 mL) was added methanesulfonyl chloride (103.0 mg; 0.903 mmol; 1.20 eq.) at 0° C. The mixture was stirred at room temperature for 15 minutes under nitrogen atmosphere. The resulting mixture was diluted with CH₂Cl₂ (50 mL), washed with brine, dried over anhydrous Na₂SO₄, filtered, and concentrated under reduced pressure. The residue was purified by flash chromatography on silica gel column using 20-60% of EtOAc in petroleum ether as eluent to provide 3-(4-(benzyloxy)phenyl)-1-(methylsulfonyl)azetidine as a white solid (140.0 mg, 58.6%). LCMS (ESI) m/z 316.1, [M−H]⁻.

### Step 4: 4-(1-(methylsulfonyl)azetidin-3-yl)phenol

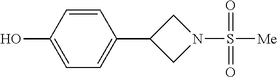 [SMILES: CS(=O)(=O)N1CC(c2ccc(O)cc2)C1]

To a solution of 3-(4-(benzyloxy)phenyl)-1-(methylsulfonyl)azetidine (140.0 mg; 0.441 mmol; 1.00 eq.) in MeOH (10 mL) was added 10% Pd/C (28.0 mg; 20% w/w) under nitrogen atmosphere. The mixture was hydrogenated at room temperature for 14 hours under hydrogen atmosphere (2 atm). The desired product was detected via LCMS. After filtration, the filtrate was concentrated under reduced pressure. The residue was purified by flash chromatography on pre-packed C18 column using 30-50% of MeCN in water (10 mmol/L. NH₄HCO₃) as eluent to provide 4-(1-(methylsulfonyl)azetidin-3-yl)phenol as a white solid (82.0 mg, 81.8%). LCMS (ESI) m/z 226.1, [M+H]⁺.

### Step 5: 4-(1-(methylsulfonyl)azetidin-3-yl)-2-nitrophenol

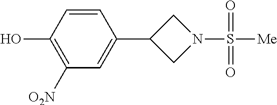 [SMILES: CS(=O)(=O)N1CC(c2ccc(O)c([N+](=O)[O-])c2)C1]

A solution of 4-(1-(methylsulfonyl)azetidin-3-yl)phenol (350.0 mg; 1.464 mmol; 1.00 eq.) and urea nitrate (180.1 mg; 1.464 mmol; 1.00 eq.) in a mixture of MeCN/water (19:1, 5 mL) was stirred at 80° C. overnight. The desired product was detected via LCMS. The mixture was concentrated under vacuum. The residue was purified by flash chromatography on pre-packed C18 column using 10-40% of MeCN in water (10 mmol/L NH₄HCO₃) as eluent to provide 4-(1-(methylsulfonyl)azetidin-3-yl)-2-nitrophenol as a black solid (200.0 mg, 50.2%). LCMS (ESI) m/z 271.0, [M+H]⁺.

### Step 6: 2-amino-4-(1-(methylsulfonyl)azetidin-3-yl)phenol

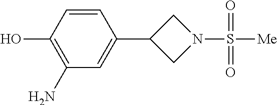 [SMILES: CS(=O)(=O)N1CC(c2ccc(O)c(N)c2)C1]

To a solution of 4-(1-(methylsulfonyl)azetidin-3-yl)-2-nitrophenol (120.0 mg; 0.441 mmol; 1.00 eq.) in MeOH (10 mL) was added 10% Pd/C (24.0 mg; 20% w/w) under nitrogen atmosphere. The mixture was hydrogenated at room temperature overnight under hydrogen atmosphere (2 atm). After filtration, the filtrate was concentrated under reduced pressure. The residue was purified by flash chromatography on silica gel column using 10-90% of EtOAc in petroleum ether as eluent to provide 2-amino-4-(1-(methylsulfonyl)azetidin-3-yl)phenol as a yellow solid (60.0 mg, 56.1%). LCMS (ESI) m/z 243.1, [M+H]⁺.

### Step 7: (N-(8-(methylamino)-5-(5-(1-(methylsulfonyl)azetidin-3-yl)benzo[d]oxazol-2-yl)-2,7-naphthyridin-3-yl)cyclopropanecarboxamide

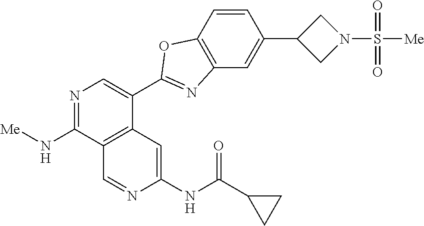 [SMILES: CNc1ncc(-c2nc3cc(C4CN(S(C)(=O)=O)C4)ccc3o2)c2cc(NC(=O)C3CC3)ncc12]

(N-(8-(methylamino)-5-(5-(1-(methylsulfonyl)azetidin-3-yl)benzo[d]oxazol-2-yl)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(1-(methylsulfonyl)azetidin-3-yl)phenol as the starting material. LCMS (ESI) m/z 493.2, [M+H]⁺. ¹H NMR (300 MHz, DMSO-d₆) δ 11.05 (s, 1H), 9.68 (s, 1H), 9.44 (s, 1H), 8.92 (s, 1H), 8.70-8.65 (m, 1H), 7.79-7.69 (m, 2H), 7.42-7.36 (m, 1H), 4.32-4.20 (m, 2H), 4.11-3.99 (m, 3H), 3.11 (s, 3H), 3.09 (d, J=4.4 Hz, 3H), 2.14-2.04 (m, 1H), 0.96-0.81 (m, 4H).

### Example 253: Synthesis of N-(8-(methylamino)-5-(5-((tetrahydro-2H-pyran-4-yl)oxy)benzo[d]oxazol-2-yl)-2,7-naphthyridin-3-yl)cyclopropanecarboxamide

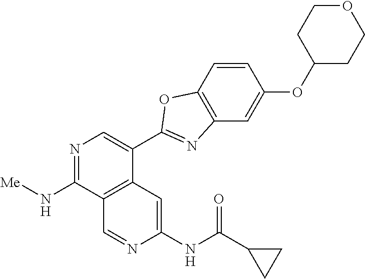 [SMILES: CNc1ncc(-c2nc3cc(OC4CCOCC4)ccc3o2)c2cc(NC(=O)C3CC3)ncc12]

### Step 1: 4-(4-(benzyloxy)phenoxy)tetrahydro-2H-pyran

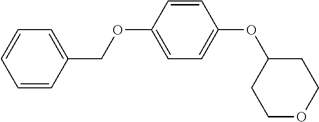 [SMILES: c1ccc(COc2ccc(OC3CCOCC3)cc2)cc1]

A solution of 4-(benzyloxy)phenol (1.00 g; 4.994 mmol; 1.00 eq.), 4-bromotetrahydro-2H-pyran (3.30 g; 19.980 mmol; 4.00 eq.) and Cs₂CO₃ (30.25 g; 9.988 mmol; 1.00 eq.) in DMF (10 mL) was stirred at 120° C. for 12 hours under nitrogen atmosphere. The desired product was detected via. LCMS. The residue was purified by flash chromatography on pre-packed C18 column using 20-100% of acetonitrile in water (10 mmol/L NH₄HCO₃) as eluent to provide 4-(4-(benzyloxy)phenoxy)tetrahydro-2H-pyran as a brown solid (588.0 mg, 41.0%). LCMS (ESI) m/z 285.1, [M+H]⁺.

### Step 2: 4-((tetrahydro-2H-pyran-4-yl)oxy)phenol

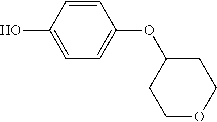 [SMILES: Oc1ccc(OC2CCOCC2)cc1]

To a solution of 4-(4-(benzyloxy)phenoxy)tetrahydro-2H-pyran (500.1 mg; 1.758 mmol; 1.00 eq.) in MeOH (10 mL) was added 10% Pd/C (150.1 mg; 30% w/w) under nitrogen atmosphere. The mixture was hydrogenated at room temperature for 3 hours under hydrogen atmosphere (2 atm). After filtration, the filtrate was concentrated under reduced pressure to afford 4-((tetrahydro-2H-pyran-4-yl)oxy)phenol as a brown solid (377.1 mg, crude). LCMS (ESI) m/z 195.1, [M+H]⁺.

### Step 3: 2-nitro-4-((tetrahydro-2H-pyran-4-yl)oxy)phenol

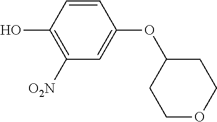 [SMILES: O=[N+]([O-])c1cc(OC2CCOCC2)ccc1O]

A solution of urea nitrate (238.8 mg; 1.941 mmol; 1.00 eq.) and 4-((tetrahydro-2H-pyran-4-yl)oxy)phenol (377.1 mg; 1.941 mmol; 1.00 eq.) in a mixture solvent of MeCN/water (20:1, 4.2 mL) was stirred at 80° C. for 1 hour under nitrogen atmosphere. The resulting mixture was diluted with water (10 mL), extracted with EtOAc (3×30 mL). The combined organic layers were washed with brine (3×10 mL), dried over anhydrous Na₂SO₄. After filtration, the filtrate was concentrated under reduced pressure. The residue was purified by flash chromatography on silica gel column using 0-10% of MeOH, in CH₂Cl₂ as eluent to provide 2-nitro-4-((tetrahydro-2H-pyran-4-yl)oxy)phenol as a red solid (230.1 mg, 50.0%). LCMS (ESI) m/z 240.1, [M+H]⁺.

### Step 4: 2-amino-4-((tetrahydro-2H-pyran-4-yl)oxy)phenol

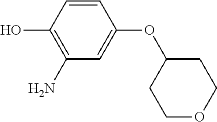 [SMILES: Nc1cc(OC2CCOCC2)ccc1O]

To a solution of 2-nitro-4-((tetrahydro-2H-pyran-4-yl)oxy)phenol (190.1 mg; 0.794 mmol; 1.00 eq.) in MeOH (5 mL) was added 10% Pd/C (190.1 mg; 100% w/w) under nitrogen atmosphere. The mixture was hydrogenated at room temperature for 3 hours under hydrogen atmosphere (2 atm). After filtration, the filtrate was concentrated under reduced pressure. The residue was purified by flash chromatography on silica gel column using 0-10% of MeOH in CH₂Cl₂ as eluent to provide 2-amino-4-((tetrahydro-2H-pyran-4-yl)oxy)phenol as a yellow solid (160.1 mg, 96.0%). LCMS (ESI) m/z 210.1, [M+H]⁺.

### Step 5: N-(8-(methylamino)-5-(5-((tetrahydro-2H-pyran-4-yl)oxy)benzo[d]oxazol-2-yl)-2,7-naphthyridin-3-yl)cyclopropanecarboxamide

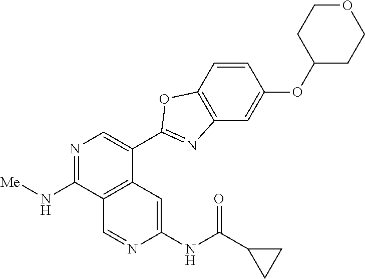 [SMILES: CNc1ncc(-c2nc3cc(OC4CCOCC4)ccc3o2)c2cc(NC(=O)C3CC3)ncc12]

N-(8-(methylamino)-5-(5-((tetrahydro-2H-pyran-4-yl)oxy)benzo[d]oxazol-2-yl)-2,7-naphthyridin-3-yl)cyclopropanecarboxamide was synthesized using a similar procedure that was previously described in Example 210, by using 3 and 2-amino-4-((tetrahydro-2H-pyran-4-yl)oxy)phenol as the starting material. LCMS (ESI) m/z 460.2, [M+H]⁺. ¹H NMR (400 MHz, DMSO-d₆) δ 11.04 (s, 1H), 9.61 (s, 1H), 944 (s, 1H), 8.89 (s, 1H), 8.70-8.57 (m, 1H), 7.62 (d, 8.8 Hz, 1H), 7.38 (d, J=2.4 Hz, 1H), 7.09-6.95 (m, 1H), 4.79-4.59 (m, 1H), 3.99-3.72 (m, 2H), 3.59-3.43 (m, 2H), 3.09 (d, J=4.4 Hz, 3H), 2.17-2.05 (m, 1H), 2.05-1.95 (m, 2H), 1.71-1.54 (m, 2H), 0.99-0.80 (m, 4H).

### Example 254: Synthesis of N-(5-(6-(hydroxymethyl)benzo[d]oxazol-2-yl)-8-(methylamino 2,7-naphthyridin-3-yl)cyclopropanecarboxamide

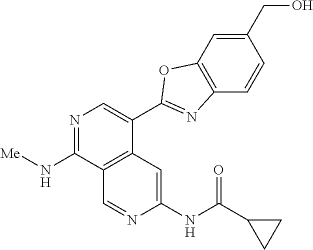 [SMILES: CNc1ncc(-c2nc3ccc(CO)cc3o2)c2cc(NC(=O)C3CC3)ncc12]

### Step 1: N-(5-(6-bromobenzo[d]oxazol-2-yl)-8-(methylamino)-2,7-naphthyridin-3-yl)cyclopropanecarboxamide

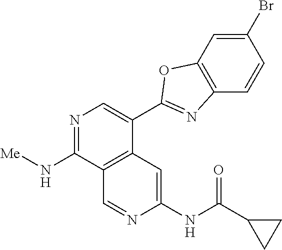 [SMILES: CNc1ncc(-c2nc3ccc(Br)cc3o2)c2cc(NC(=O)C3CC3)ncc12]

N-(5-(6-bromobenzo[d]oxazol-2-yl)-8-(methylamino)-2,7-naphthyridin-3-yl)cyclopropanecarboxamide was synthesized using a similar procedure that was previously, described in Example 210, by using N-(5-formyl-8-(methylamino)-2,7-naphthyridin-3-yl)cyclopropanecarboxamide and 2-amino-5-bromophenol as the starting material. LCMS (ESI) m/z 438.0, [M+H]⁺.

### Step 2: N-(5-(6-(hydroxymethyl)benzo[d]oxazol-2-yl-8-ethylamino)-2,7-naphthyridin-3-yl)cyclopropanecarboxamide

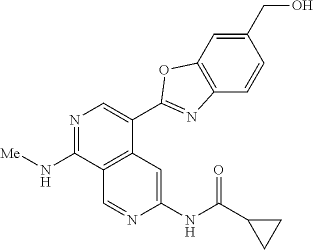 [SMILES: CNc1ncc(-c2nc3ccc(CO)cc3o2)c2cc(NC(=O)C3CC3)ncc12]

To a solution of N-(5-(6-bromobenzo[d]oxazol-2-yl)-8-(methylamino)-2,7-naphthyridin-3-yl)cyclopropanecarboxamide (176.0 mg; 0.402 mmol; 1.00 eq.) in 1,4-dioxane were added Pd(PPh₃)₄ (46.4 mg; 0.040 mmol; 0.10 eq.) and (tributylstannyl)methanol (389.4 mg; 1.214 mmol; 3.02 eq.) under nitrogen atmosphere. The mixture was stirred at 90 for 14 hours. The reaction mixture was cooled to room temperature and concentrated under reduced pressure. The residue was purified by flash chromatography on silica gel column using 0-8% of MeOH in CH₂Cl₂ as eluent to provide a crude product. The crude product was purified by flash chromatography on pre-packed C18 column using 0-75% of MeOH in water (10 mmol/L NH₄HCO₃) to provide N-(5-(6-(hydroxymethyl)benzo[d]oxazol-2-yl)-8-(methylamino)-2,7-naphthyridin-3-yl)cyclopropanecarboxamide as a yellow solid (6.0 mg, 3.8%). LCMS (ESI) m/z 390.1, [M+H]⁺. ¹H NMR (400 MHz, DMSO-d₆) δ 11.03 (s, 1H), 9.67 (s, 1H), 9.44 (s, 1H), 8.92 (s, 1H), 8.70-8.57 (m, 1H), 7.74-7.61 (m, 2H), 7.34 (d, J=8.0 Hz, 1H), 5.34 (t, J=5.6 Hz, 1H), 4.66 (d, J=5.6 Hz, 2H), 3.10 (d, J=4.4 Hz, 3H), 2.16-2.05 (m, 1H), 1.01-0.76 (m, 4H).

### Example 255: Synthesis of N-(8-(methylamino)-5-(5-(2,2,2-trifluoroethyl)benzo[d]oxazol-2-yl)-2,7-naphthyridin-3-yl)cyclopropanecarboxamide

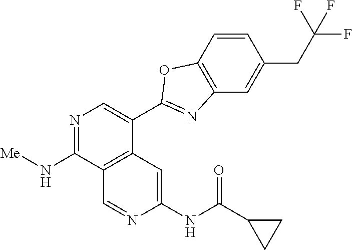 [SMILES: CNc1ncc(-c2nc3cc(CC(F)(F)F)ccc3o2)c2cc(NC(=O)C3CC3)ncc12]

### Step 1: 2-(4-(benzyloxy)-3-nitrophenyl)-4,4,5,5-tetramethyl-1,3,2-dioxaborolane

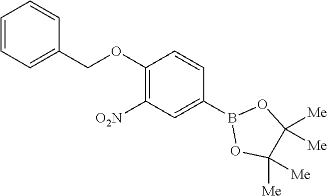 [SMILES: CC1(C)OB(c2ccc(OCc3ccccc3)c([N+](=O)[O-])c2)OC1(C)C]

To a stirred solution of 1-(benzyloxy)-4-promo-2-nitrobenzene (2.00 g; 6.491 mmol; 1.00 eq.) and 4,4,4′,4′,5,5,5′,5′-octamethyl-2,2′-bi(1,3,2-dioxaborolane) (1.81 g; 7.140 mmol; 1.10 eq.) in 1,4-dioxane (5 mL) were added Pd(dppf)C₁₋₂ (0.47 g; 0.649 mmol; 0.10 eq.) and KOAc (1.27 g; 12.982 mmol; 2.00 eq.). The resulting mixture was stirred at 100° C. for 3 hours under nitrogen atmosphere. The desired product was detected via LCMS. The mixture was allowed to cool to room temperature and concentrated under reduced pressure. The residue was purified by flash chromatography on silica gel column using 10-30% of CH₂Cl₂ in petroleum ether as eluent to provide 2-(4-(benzyloxy)-3-nitrophenyl)-4,4,5,5-tetramethyl-1,3,2-dioxaborolane as a colorless oil (1.20 g, 52.1%). LCMS (ESI) m/z 356.2, [M+H]⁺.

### Step 2: 1-(benzyloxy)-2-nitro-4-(2,2,2-trifluoroethyl)benzene

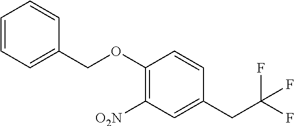 [SMILES: O=[N+]([O-])c1cc(CC(F)(F)F)ccc1OCc1ccccc1]

To a stirred solution of 2-(4-(benzyloxy)-3-nitrophenyl)-4,4,5,5-tetramethyl-1,3,2-dioxaborolane (1.00 g; 2.822 mmol; 1.00 eq.) and 1,1,1-trifluoro-2-iodoethane (650.0 mg, 3.100 mmol; 1.10 eq.) in a mixture solvent of 1,4-dioxane/water (5:1, 10 mL) were added XPhos Pd G₃ (238.2 mg; 0.282 mmol; 0.10 eq.), XPhos (268.3 mg; 0.564 mmol; 0.20 eq.) and K₃PO₄ (10.20 g; 5.644 mmol; 2.00 eq.). The resulting mixture was stirred at 90° C. for 1 hour under nitrogen atmosphere. The desired product was detected via LCMS. The mixture was allowed to cool to room temperature. The resulting mixture was concentrated under reduced pressure. The residue was purified by flash chromatography on pre-packed C18 column using 20-60% of MeCN in water (10 mmol/L NH₄HCO₃) as eluent to provide 1-(benzyloxy)-2-nitro-4-(2,2,2-trifluoroethyl)benzene as an off-white solid (120 mg, 13.7%). LCMS (ESI) m/z 312.1, [M+H]⁺.

### Step 3: 2-amino-4-(2,2,2-trifluoroethyl)phenol

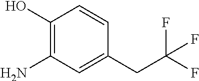 [SMILES: Nc1cc(CC(F)(F)F)ccc1O]

To a solution of 1-(benzyloxy)-2-nitro-4-(2,2,2-trifluoroethyl)benzene (180.0 mg; 0.578 mmol; 1.00 eq.) in MeOH (6 mL) was added 10% Pd/C (60.0 mg; 33% w/w) under nitrogen atmosphere. The mixture was hydrogenated at room temperature for 1 hour under hydrogen atmosphere (2 atm). The desired product was detected via LCMS. The resulting mixture was filtered, the filter cake was washed with MeOH. The filtrate was concentrated under reduced pressure to afford 2-amino-4-(2,2,2-trifluoroethyl)phenol as a light yellow oil (100.0 mg, 90.5%). LCMS (ESI) m/z 192.1, [M+H]⁺.

### Step 4: N-(8-(methylamino)-5-(5-(2,2,2-trifluoroethyl)benzo[d]oxazol-2-yl)-2,7-naphthyridin-3-yl)cyclopropanecarboxamide

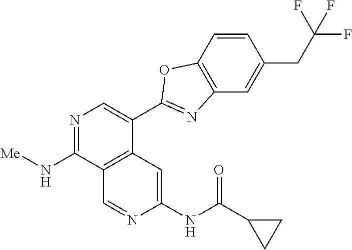 [SMILES: CNc1ncc(-c2nc3cc(CC(F)(F)F)ccc3o2)c2cc(NC(=O)C3CC3)ncc12]

N-(8-(methylamino)-5-(5-(2,2,2-trifluoroethyl)benzo[d]oxazol-2-yl)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(2,2,2-trifluoroethyl)phenol as the starting material. LCMS (ESI) m/z 442.1, [M+H]⁺. ¹H NMR (400 MHz, DMSO-d₆) δ 11.05 (s, 1H), 9.74 (s, 1H), 9.45 (s, 1H), 8.93 (s, 1H), 8.72-8.64 (m, 1H), 7.80-7.71 (m, 2H), 7.40-7.34 (m, 1H), 3.90-3.73 (m, 2H), 3.10 (d, J=4.4 Hz, 3H), 2.16-2.06 (m, 1H), 0.97-0.79 (m, 4H).

### Example 256: Synthesis of N-(5-(5-(2-methoxy-2-methylpropoxy)benzo[d]oxazol-2-yl)-8-(methylamino)-2,7-naphthyridin-3-yl)cyclopropanecarboxamide

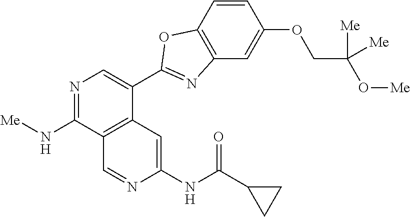 [SMILES: CNc1ncc(-c2nc3cc(OCC(C)(C)OC)ccc3o2)c2cc(NC(=O)C3CC3)ncc12]

### Step 1: 1-4-(benzyloxy)phenoxy)-2-methylpropan-2-ol

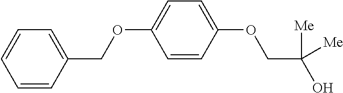 [SMILES: CC(C)(O)COc1ccc(OCc2ccccc2)cc1]

A solution of 4-(benzyloxy)phenol (1.00 g; 4.99 mmol; 1.00 eq.), 1-promo-2-methylpropan-2-ol (1.15 g; 7,491 mmol; 1.50 eq.) and Cs₂CO₃ (3.25 g; 9.98 mmol; 2.00 eq.) in DMF (10 mL) was stirred at 120° C. for 14 hours. The desired product was detected via LCMS. The resulting mixture was diluted with water (100 mL) to precipitate solids. The precipitated solids were collected by filtration and washed with water (2×10 mL) to provide 1-(4-(benzyloxy)phenoxy)-2-methylpropan-2-ol as a white solid (1.31 g, 96.1%). LCMS (ESI) m/z 271.1, [M−H]⁻.

### Step 2: 1-(benzyloxy)-4-(2-methoxy-2-methylpropoxy)benzene

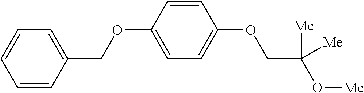 [SMILES: COC(C)(C)COc1ccc(OCc2ccccc2)cc1]

To a solution of 1-(4-(benzyloxy)phenoxy)-2-methylpropan-2-ol (1.20 g; 4.406 mmol; 1.00 eq.) in DMF (12 mL) was added NaH (60%) (441.0 mg; 11,012 mmol; 2.50 eq.) at 0° C. under nitrogen atmosphere. After stirring at room temperature for 0.5 hour, to the above solution was added MeI (1.25 g; 8.812 mmol; 2.00 eq.). The resulting solution was stirred at room temperature for 3 hours. The desired product was detected via LCMS. The reaction was quenched with saturated NH₄Cl solution (20 mL), extracted with EtOAc (3×50 mL). The combined organic layers were washed 13 with brine (2×20 mL), dried over Na₂SO₄. After filtration, the filtrate was concentrated under reduced pressure. The residue was purified by flash chromatography on pre-packed C18 column using 20-50% of MeCN in water (10 mmol/L NH₄HCO₃) as eluent to provide 1-(benzyloxy)-4-(2-methoxy-2-methylpropoxy)benzene as a yellow oil (1.20 g, 95.1%). LCMS (ESI) m/z 285.2, [M−H]⁻.

### Step 3: 4-(2-methoxy-2-methylpropoxy)phenol

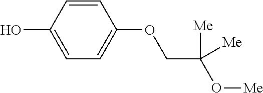 [SMILES: COC(C)(C)COc1ccc(O)cc1]

To a solution of 1-(benzyloxy)-4-(2-methoxy-2-methylpropoxy)benzene (1.20 g; 4.190 mmol; 1.00 eq.) in MeOH (15 mL) was added 10% Pd/C (600 mg; 50% w/w) under nitrogen atmosphere. The mixture was hydrogenated at room temperature overnight under hydrogen atmosphere (2 atm). The desired product was detected via. LCMS, After filtration, the filtrate was concentrated under reduced pressure. The residue was purified by flash chromatography on pre-packed C18 column using 30-50% of MeCN in water (10 mmol/L NH₄HCO₃) as eluent to provide 4-(2-methoxy-2-methylpropoxy)phenol as a light yellow oil (779.0 mg, 94.2%). LCMS (ESI) m/z 195.1, [M−H]⁻.

### Step 4: 4-(2-methoxy-2-methylpropoxy)-2-nitrophenol

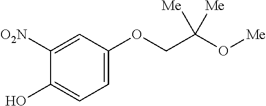 [SMILES: COC(C)(C)COc1ccc(O)c([N+](=O)[O-])c1]

A solution of 4-(2-methoxy-2-methylpropoxy)phenol (350.0 mg; 1.783 mmol; 1.00 eq.) and urea nitrate (219.6 mg; 1.784 mmol, 1.00 eq.) in a mixture solvent of MeCN/water (19:1, 10 mL) was stirred at 80° C. for 14 hours under nitrogen atmosphere. The desired product was detected via LCMS. The resulting mixture was concentrated under reduced pressure. The residue was purified by flash chromatography on silica gel column using 25-35% of EtOAc in petroleum ether as eluent to provide 4-(2-methoxy-2-methylpropoxy)-2-nitrophenol as a yellow solid (360.4 mg, 83.1%). LCMS (ESI) m/z 240.1, [M−H]⁻.

### Step 5: 2-amino-4-(2-methoxy-2-methylpropoxy)phenol

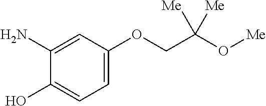 [SMILES: COC(C)(C)COc1ccc(O)c(N)c1]

To a solution of 4-(2-methoxy-2-methylpropoxy)-2-nitrophenol (180.0 mg; 0.746 mmol; 1.00 eq.) in MeOH (10 mL) was added 10% Pd/C (180.0 mg; 100% w/w) under nitrogen atmosphere. The mixture was hydrogenated at room temperature for 1 hour under hydrogen atmosphere (2 atm), The desired product was detected via LCMS. After filtration, the filtrate was concentrated under reduced pressure to provide 2-amino-4-(2-methoxy-2-methylpropoxy)phenol as a brown solid (157.1 mg, crude). LCMS (ESI) m/z 212.1, [M+H]⁺.

### Step 6: N-(5-(5-(2-methoxy-2-methylpropoxy)benzo[d]oxazol-2-yl)-8-(methylamino)-2,7-naphthyridin-3-yl)cyclopropanecarboxamide

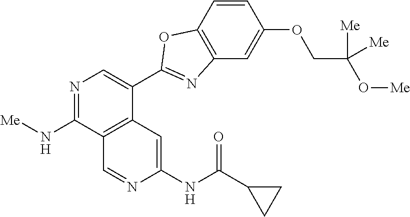 [SMILES: CNc1ncc(-c2nc3cc(OCC(C)(C)OC)ccc3o2)c2cc(NC(=O)C3CC3)ncc12]

N-(5-(5-(2-methoxy-2-methylpropoxy)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(2-methoxy-2-methylpropoxy)phenol as the starting material. LCMS (ESI) m/z 462.2, [M+H]⁺. ¹H MIR (400 MHz, DMSO-d₆) δ 11.03 (s, 1H), 9.66 (s, 1H), 9.44 (s, 1H), 8.89 (s, 1H), 8.66-8.61 (m, 1H), 7.61 (d, J=8.8 Hz, 1H), 7.30 (d, J=2.4 Hz, 1H), 6.98 (dd, J=8.8, 2.4 Hz, 1H), 3.94 (s, 2H), 3.20 (s, 3H), 3.09 (d, J=4.4 Hz, 3H), 2.14-2.06 (m, 1H), 1.25 (5, 6H), 0.96-0.81 (m, 4H).

### Example 257: Synthesis of N-(8-(methylamino)-5-(5-((methylsulfonyl)methyl)benzo[d]oxazol-2-yl)-2,7-naphthyridin-3-yl)cyclopropanecarboxamide

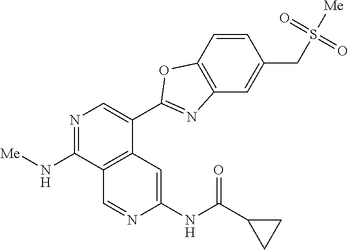 [SMILES: CNc1ncc(-c2nc3cc(CS(C)(=O)=O)ccc3o2)c2cc(NC(=O)C3CC3)ncc12]

### Step 1: 4-(benzyloxy)-3-nitrobenzaldehyde

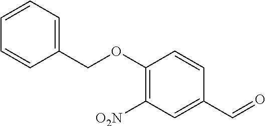 [SMILES: O=Cc1ccc(OCc2ccccc2)c([N+](=O)[O-])c1]

To a stirred solution of 4-hydroxy-3-nitrobenzaldehyde (5.00 g; 29,914 mmol; 1.00 eq.) and K₂CO₃ (6.11 g; 44.785 mmol; 1.50 eq.) in DMF (50 mL) was added (bromomethyl)benzene (6.61 g; 38.644 mmol; 1.30 eq.) at 0° C. The resulting solution was stirred at 65° C. overnight under nitrogen atmosphere. The desired product was detected via LCMS. The resulting mixture was diluted with water (100 mL) to precipitate solids. The precipitated solids were collected by filtration and washed with water (10 mL) just once to afford 4-(benzyloxy)-3-nitrobenzaldehyde as a yellow solid (6.20 g, 80.5%). LCMS (ESI) m/z 258.1, [M+H]⁺.

### Step 2: (4-(benzyloxy)-3-nitrophenyl)methanol

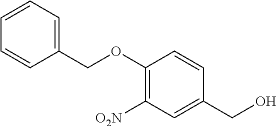 [SMILES: O=[N+]([O-])c1cc(CO)ccc1OCc1ccccc1]

To a stirred solution of 4-(benzyloxy)-3-nitrobenzaldehyde (4.01 g; 15.541 mmol; 1.00 eq.) in THF (40 mL) was added NaBH₄ (588.1 mg; 15.541 mmol; 1.00 eq.) in portions at 0° C. The mixture was stirred at room temperature for 1 hour under nitrogen atmosphere. The desired product was detected via LCMS. The reaction was quenched with water (10 mL) and extracted with EtOAc (3 50 mL). The combined organic layers were washed with brine (2×50 mL) and dried over anhydrous Na₂SO₄. After filtration, the filtrate was concentrated under reduced pressure to afford (4-(benzyloxy)-3-nitrophenyl)methanol as a yellow solid (2.70 g, 66.9%). LCMS (ESI) m/z 260, 1, [M+H]⁺.

### Step 3: 4-(benzyloxy)-3-nitrobenzyl methanesulfonate

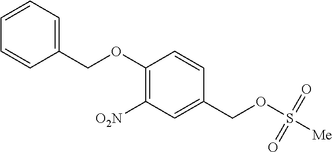 [SMILES: CS(=O)(=O)OCc1ccc(OCc2ccccc2)c([N+](=O)[O-])c1]

To a stirred solution of (4-(benzyloxy)-3-nitrophenyl)methanol (800.1 mg; 3.082 mmol; 1.00 eq.) and DIPEA (956.3 mg 7.39 mmol; 2.50 eq.) in CH₂Cl₂ (10 mL) was added methanesulfonic anhydride (1.18 g; 6,774 mmol; 2.00 eq.) at 0° C. The resulting mixture was stirred at room temperature for 2 hours under nitrogen atmosphere. The desired product was detected via LCMS. The mixture was diluted with CH₂Cl₂ (20 mL) and washed with water (2×5 mL). The organic layer was dried over anhydrous-Na₂SO₄. After filtration, the filtrate was concentrated under reduced pressure to afford 4-(benzyloxy)-3-nitrobenzyl methanesulfonate as a yellow oil (900.2 mg, 86.4%). LCMS (ESI) m/z 338.1, [M+H]⁺.

### Step 4: 1-(benzyloxy)-4-((methylsulfonyl)methyl)-2-nitrobenzene

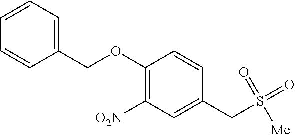 [SMILES: CS(=O)(=O)Cc1ccc(OCc2ccccc2)c([N+](=O)[O-])c1]

A solution of 4-(benzyloxy)-3-nitrobenzyl methanesulfonate (900.2 mg; 2.656 mmol; 1.00 eq.) and sodium methanesulfinate (360.3 mg; 3.524 mmol, 1.30 eq.) in NMP (3 mL) was stirred at 80° C. for 2 hours under nitrogen atmosphere. The desired product was detected via LCMS. The reaction was diluted with water (20 mL) and extracted with EtOAc (3×10 mL). The combined organic layers were washed with brine (3×10 mL) and dried over anhydrous-Na₂SO₄, After filtration, the filtrate was concentrated under reduced pressure to afford 1-(benzyloxy)-4-((methylsulfonyl)methyl)-2-nitrobenzene as a yellow solid (400.2 mg, 46.6%). LCMS (ESI) m/z 322.1 [M+H]⁺.

### Step 5: 2-amino-4-((methylsulfonyl)methyl)phenol

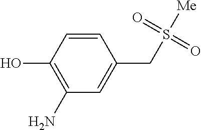 [SMILES: CS(=O)(=O)Cc1ccc(O)c(N)c1]

To a solution of 1-(benzyloxy)-4-(methanesulfonylmethyl)-2-nitrobenzene (80.3 mg; 0.240 mmol; 1.00 eq.) in Meth (5 mL) was added 10% Pd/C (25.3 mg; 30% w/w) under nitrogen atmosphere. The mixture was hydrogenated at 40° C. for 1 hour under hydrogen atmosphere (2 atm). The resulting mixture was filtered, the filter cake was washed with MeOH. The filtrate was concentrated under reduced pressure to afford 2-amino-4-((methylsulfonyl)methyl)phenol as a colorless oil (30.2 mg, 59.8%). LCMS (ESI) m/z 202.0, [M+H]⁺.

### Step 6: N-(8-(methylamino)-5-(5-((methylsulfonyl)methyl)benzo[d]oxazol-2-yl)-2,7-naphthyridin-3-yl)cyclopropanecarboxamide

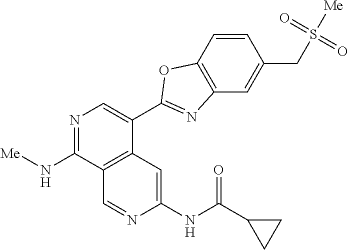 [SMILES: CNc1ncc(-c2nc3cc(CS(C)(=O)=O)ccc3o2)c2cc(NC(=O)C3CC3)ncc12]

N-(8-(methylamino)-5-(5-((methylsulfonyl)methyl)benzo[d]oxazol-2-yl)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-((methylsulfonyl)methyl)phenol as the starting material. LCMS (ESI) m/z 452.1, [M+H]⁺. ¹H NMR (400 MHz, DMSO-d₆) δ 11.07 (s, 1H), 9.79 (s, 1H), 9.46 (s, 1H), 8.93 (s, 1H), 8.79-8.71 (m, 1H), 7.84 (d, J=1.2 Hz, 1H), 7.77 (d, J=8.4 Hz, 1H), 7.43 (dd, J==8.4, 1.2 Hz, 1H), 4.65 (s, 2H), 3.10 (d, J=4.4 Hz, 3H), 2.92 (s, 3H), 2.15-2.06 (m, 1H), 0.94-0.84 (m, 4H).

### Example 258: Synthesis of N-(8-(methylamino)-5-(5-(oxetan-3-yl)benzo[d]oxazol-2-yl)-2,7-naphthyridin-3-yl)cyclopropanecarboxamide

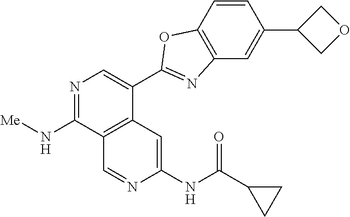 [SMILES: CNc1ncc(-c2nc3cc(C4COC4)ccc3o2)c2cc(NC(=O)C3CC3)ncc12]

### Step 1: 3-(4-(benzyloxy)phenyl)oxetane

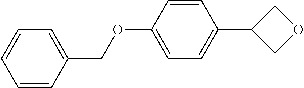 [SMILES: c1ccc(COc2ccc(C3COC3)cc2)cc1]

To a solution of (4-(benzyloxy)phenyl)boronic acid (1.00 g; 4.385 mmol; 2.20 eq.) in i-PrOH (20 mL) was added nickel(I) iodide (0.14 g; 0.439 mmol; 0.20 eq.), (1R,2R)-2-aminocyclohexan-1-ol (0.05 g; 0.439 mmol; 0.20 eq.) and NaHMDS (2.19 mL; 4.385 mmol; 2.20 eq.) dropwise at room temperature. To the above solution was added 3-iodooxetane (0.40 g; 2.192 mmol; 1.00 eq.). The resulting solution was stirred at 80° C. for 3 hours under nitrogen atmosphere. The resulting mixture was quenched with water (10 mL), extracted with CH₂Cl₂ (3×40 mL). The combined organic layers were washed with brine (2×10 mL), dried over anhydrous Na₂SO₄. After filtration, the filtrate was concentrated under reduced pressure. The residue was purified by flash chromatography on silica gel column using 0-20% of EtOAc in petroleum ether as eluent to provide 3-(4-(benzyloxy)phenyl)oxetane as a white solid (260.2 mg, 34.9%). LCMS (ESI) m/z 241.0, [M+H]⁺.

### Step 2: 4-(oxetan-3-yl)phenol

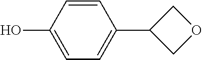 [SMILES: Oc1ccc(C2COC2)cc1]

To a solution of 3-(4-(benzyloxy)phenyl)oxetane (260.2 mg; 1.082 mmol; 1.00 eq.) in a mixture solvent of DMA/MeOH (1:1, 4 mL) was added 10% Pd/C (78.2 mg; 30% w/w) under nitrogen atmosphere. The mixture was hydrogenated at room temperature for 2 hours under hydrogen atmosphere (2 atm). After filtration, the filtrate was concentrated under reduced pressure. The resulting mixture was diluted with EtOAc (30 mL), washed with brine (3×5 mL). The organic layer was dried over anhydrous-Na₂SO₄. After filtration, the filtrate was concentrated under reduced pressure. The residue was purified by flash chromatography on silica gel column using 15-50% of EtOAc in petroleum ether as eluent to provide 4-(oxetan-3-yl)phenol as a white solid (130.1 mg, 80.0%). LCMS (ESI) m/z 151.0, [M+H]⁺.

### Step 3: 2-nitro-4-(oxetan-3-yl)phenol

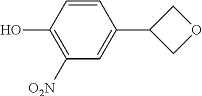 [SMILES: O=[N+]([O-])c1cc(C2COC2)ccc1O]

A solution of urea nitrate (81.9 mg; 0.666 mmol; 1.00 eq.) and 4-(oxetan-3-yl)phenol (100.1 mg; 0.666 mmol; 1.00 eq.) in a mixture solvent of MeCN/water (20:1, 4.2 mL) was stirred at 80° C. for 1 hour under nitrogen atmosphere. The resulting mixture was diluted with EtOAc (30 mL), washed with brine (2×10 mL), dried over anhydrous Na₂SO₄, filtered, and concentrated under reduced pressure. The resulting mixture was concentrated under reduced pressure. The residue was purified by flash chromatography on silica gel column using 10-40% of EtOAc in petroleum ether as eluent to provide 2-nitro-4-(oxetan-3-yl)phenol (100.0 mg, 76.9%) as an off-white solid. LCMS (ESI) m/z 196.1, [M+H]⁺.

### Step 4: 2-amino-4-(oxetan-3-yl)phenol

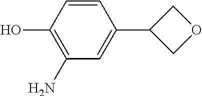 [SMILES: Nc1cc(C2COC2)ccc1O]

To a solution of 2-nitro-4-(oxetan-3-yl)phenol (100.0 mg; 0.512 mmol; 1.00 eq.) in MeOH (5 ml) was added 10% Pd/C (100.0 mg; 100% w/w) under nitrogen atmosphere. The mixture was hydrogenated at room temperature for 3 hours under hydrogen atmosphere (2 atm). After filtration, the filtrate was concentrated under reduced pressure to afford 2-amino-4-(oxetan-3-yl)phenol as a yellow solid (75 mg, crude). LCMS (ESI) m/z 166.1, [M+H]⁺.

### Step 5: N-(8-(methylamino)-5-(5-(oxetan-3-yl)benzo[d]oxazol-2-yl)-2,7-naphthyridin-3-yl)cyclopropanecarboxamide

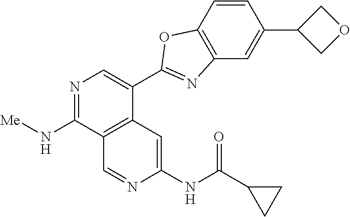 [SMILES: CNc1ncc(-c2nc3cc(C4COC4)ccc3o2)c2cc(NC(=O)C3CC3)ncc12]

N-(8-(methylamino)-5-(5-(oxetan-3-yl)benzo[d]oxazol-2-yl)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(oxetan-3-yl)phenol as the starting material. LCMS (ESI) m/z 416.2, [M+H]⁺. ¹H NMR (400 MHz, DMSO-d₆) δ 11.04 (s, 1H), 9.71 (s, 1H), 9.45 (s, 1H), 8.93 (s, 1H), 8.70-8.58 (m, 1H), 7.79 (d, J=2.4 Hz, 1H), 7.72 (d, J=8.0 Hz, 1H), 7.48-7.36 (m, 1H), 5.09-4.91 (m, 2H), 4.80-4.62 (m, 2H), 4.50-4.33 (m, 1H), 3.10 (d, J=4.4 Hz, 314), 2.14-2.09 (m, 1H), 0.97-0.89 (m, 2H), 0.89-0.80 (m, 2H).

### Example 259: Synthesis of N-(5-(6-methyl-5-(2-methylmorpholino)benzo[d]oxazol-2-yl)-8-(methylamino)-2,7-naphthyridin-3-yl)cyclopropanecarboxamide

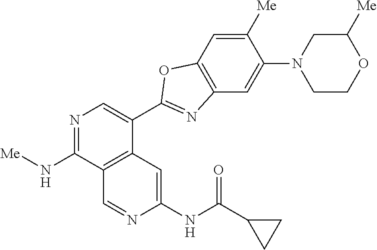 [SMILES: CNc1ncc(-c2nc3cc(N4CCOC(C)C4)c(C)cc3o2)c2cc(NC(=O)C3CC3)ncc12]

### Step 1: N-(5-(5-bromo-6-methylbenzo[d]oxazol-2-yl)-8-(methylamino)-2,7-naphthyridin-3-yl)cyclopropanecarboxamide

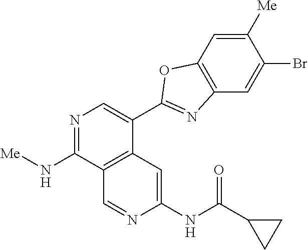 [SMILES: CNc1ncc(-c2nc3cc(Br)c(C)cc3o2)c2cc(NC(=O)C3CC3)ncc12]

A solution of N-(5-formyl-8-(methylamino)-2,7-naplathyridin-3-yl)cyclopropanecarboxamide (300.0 mg, 1.110 mmol; 1.00 eq.) and 2-amino-4-bromo-5-methylphenol (224.3 mg; 1.110 mmol; 1.00 eq.) in a mixture solvent of toluene/DMSO (5:1, 36 mL) was stirred at 110° C. for 12 hours. The resulting mixture was concentrated under reduced pressure to remove toluene. To the mixture was added CH₂Cl₂ (20 mL) and IDI). (277.1 mg; 1.221 mmol; 1.10 eq.). The resulting solution was stirred at room temperature for 2 hours under nitrogen atmosphere. LC-MS indicated that the starting material was consumed. The mixture was concentrated under reduced pressure. The residue was purified by flash chromatography on pre-packed. C18 column using 30-40% of MeOH in water (10 mmol/L NH₄HCO₃) as eluent to provide N-(5-(5-bromo-6-methylbenzo[d]oxazol-2-yl)-8-(methylamino)-2,7-naphthyridin-3-yl)cyclopropanecarboxamide as a light brown solid (470.0 mg, 93.6%). LCMS (ESI) m/z 452.1, [M+H]⁺.

### Step 2: N-(5-(6-methyl-5-(2-methylmorpholino)benzo[d]oxazol-2-yl)-8-(methylamino)-2,7-naphthyridin-3-yl)cyclopropanecarboxamide

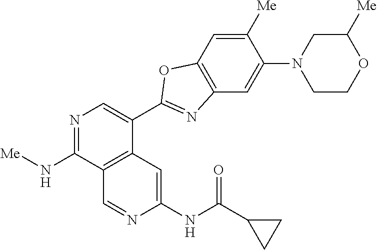 [SMILES: CNc1ncc(-c2nc3cc(N4CCOC(C)C4)c(C)cc3o2)c2cc(NC(=O)C3CC3)ncc12]

A stirring mixture of N-(5-(5-bromo-6-methylbenzo[d]oxazol-L-yl)-8-(methylamino)-2,7-naphthyridin-3-yl)cyclopropanecarboxamide (100.0 mg; 0.221 mmol; 1.00 eq.) and 2-amino-4-bromo-5-methylphenol (67.0 mg; 0.662 mmol; 3.00 eq.) in 1,4-dioxane (10 mL). To the mixture were added EPhos (24.0 mg; 0.045 mmol; 0.20 eq.), EPhos Pd G₄ (41.1 mg; 0.045 mmol; 0.20 eq.) and Cs₂CO₃ (288.1 mg; 0.884 mmol; 4.00 eq.). The resulting solution was stirred at 100° C. overnight under nitrogen atmosphere. The desired product was detected via LCMS. The mixture was concentrated under reduced pressure. The residue was purified by flash chromatography on silica gel column using 5-10% of MeOH in CH₂Cl₂ as eluent to provide a crude product. The residue was purified by flash chromatography on pre-packed C18 column using 30-40% of MeOH in water (10 mmol/L NH₄HCO₃) as eluent to provide N-(5-(6-methyl-5-(2-methylmorpholino)benzo[d]oxazol-2-yl)-8-(methylamino)-2,7-naphthyridin-3-yl)cyclopropanecarboxamide as a yellow solid (7.3 mg, 6.9%). LCMS (ESI) m/z 473.2, [M+H]⁺. ¹H NMR (400 MHz, DMSO-d₆) (δ 11.03 (s, 1H), 9.64 (s, 1H), 9.43 (s, 1H), 8.89 (s, 1H), 8.68 8.56 (m, 1H), 7.55 (s, 1H), 7.38 (s, 1H), 3.94-3.86 (m, 1H), 3.79-3.70 (m, 2H), 3.09 (d, J=4.4 Hz, 3H), 3.02-2.88 (m, 2H), 2.84-2.75 (m, 1H), 2.50 (s, 1H), 2.44-2.40 (m, 3H), 2.15-2.06 (m, 1H), 1.16 (d, J=6.0 HZ, 3H), 0.96-0.83 (m, 4H).

### Example 260: Synthesis of N-(5-(5-(1-(4-hydroxy-3-methyltetrahydrofuran-3-yl)piperidin-4-yl)benzo[d]oxazol-2-yl)-8-(methylamino)-2,7-naphthyridin-3-yl)cyclopropanecarboxamide (cis racemate)

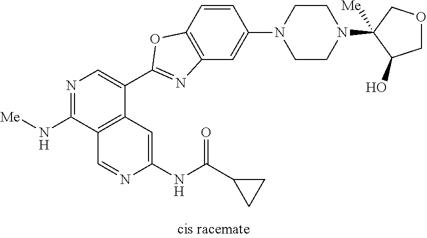 [SMILES: CNc1ncc(-c2nc3cc(N4CCN([C@@]5(C)COC[C@H]5O)CC4)ccc3o2)c2cc(NC(=O)C3CC3)ncc12]

### Step 1: tetrahydrofuran-3,4-dial (trans racemate)

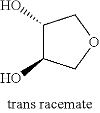

A solution of 3,6-dioxabicyclo[3.1.0]hexane (10.00 g; 116.158 mmol; 1.00 eq.) and H₂SO₄ (36.00 g; 367.085 mmol; 3.16 eq.) in water (200 mL) was stirred at 100° C. overnight under nitrogen atmosphere. The desired product was detected via LCMS. The mixture was allowed to cool down to room temperature. The resulting mixture was concentrated under reduced pressure. The residue was dissolved with THF (100 mL), and the insoluble solids were filtered out. The filtrate was concentrated under reduced pressure to afford tetrahydrofuran-3,4-diol (trans racemate) as a brown oil (12.0 g, 99.2%). LCMS (ESI) m/z 105.0, [M+H]⁺.

### Step 2: 4-((tert-butyldiphenylsilyl)oxy)tetrahydrofuran-3-ol (trans racemate)

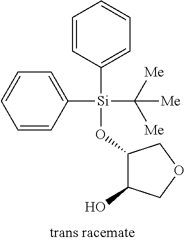 [SMILES: CC(C)(C)[Si](O[C@@H]1COC[C@H]1O)(c1ccccc1)c1ccccc1]

To a solution of tetrahydrofuran-3,4-diol (trans racemate) (12.00 g; 115.384 mmol; 1.00 eq.) in MeCN (300 mL) were added ice-imidazole (11.78 g; 173.076 mmol; 1.50 eq.) and tert-butylchlorodiphenylsilane (31.71 g; 115.384 mmol; 1.01 eq.) at room temperature. The mixture was stirred at 70° C. overnight under nitrogen atmosphere. The desired product was detected via LCMS. The mixture was allowed to cool down to room temperature. The resulting mixture was concentrated under reduced pressure. The residue was dissolved in EtOAc (500 mL) and washed with brine (2×500 mL). The organic layers were dried over anhydrous Na₂SO₄, filtered, and concentrated under reduced pressure. The residue was purified by flash chromatography on silica gel column using 25-60% of EtOAc in petroleum ether as eluent to afford 4-((tert-butyldiphenylsilyl)oxy)tetrahydrofuran-3-01 (trans racemate) as a white solid (28.00 g, 71.6%). LCMS (ESI) m/z 343.2, [M+H]⁺.

### Step 3: 4-tert-butyldiphenylsilyl)oxy)dihydrofuran-3(2H)-one

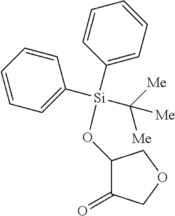 [SMILES: CC(C)(C)[Si](OC1COCC1=O)(c1ccccc1)c1ccccc1]

A stirring mixture of 4-((tart-butyldiphenylsilyl)oxy)tetrahydrofuran-3-ol (trans racemate) (28.00 g; 81,749 mmol; 1.00 eq.) and Dess-Martin (40.00 g; 94.308 mmol; 1.15 eq.) in CH₂Cl₂ (280 mL) was stirred at room temperature for 3 hours under nitrogen atmosphere. The desired product was detected via LCMS. The reaction was quenched by the addition of saturated NaHCO₃ solution (200 mL), extracted with CH₂Cl₂ (2×200 mL). The combined organic layers were washed with water (2×50 mL) and dried over anhydrous Na₂SO₄. After filtration, the filtrate was concentrated under reduced pressure. The residue was purified by flash chromatography on silica gel column using 25-60% of EtOAc in petroleum ether as eluent to afford 4-((tert-butyldiphenylsilyl)oxy)dihydrofuran-3(2H)-one as a colorless oil (22.00 g, 79.0%), LCMS (ESI) m/z 341.1, [M+H]⁺.

### Step 4: tert-butyl 4-(4-hydroxyphenyl)piperidine-1-carboxylate

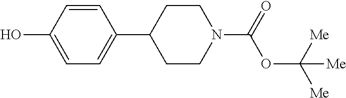 [SMILES: CC(C)(C)OC(=O)N1CCC(c2ccc(O)cc2)CC1]

To a solution of 4-(piperidin-4-yl)phenol hydrochloride (9.00 g; 42.113 mmol; 1.00 eq.) and DIPEA (17.00 g; 131.531 mmol; 3.12 eq.) in CH₂Cl₂ (100 mL) was added (Boc)₂O (11.00 g; 50.401 mmol; 1.20 eq.) at room temperature and stirred for 2 hours. The desired product was detected via LCMS. The mixture was diluted with CH₂Cl₂ (200 mL) and washed with water (2 100 mL). The organic layer was dried over anhydrous Na₂SO₄, filtered, and concentrated under reduced pressure. The residue was purified by flash chromatography on silica gel column using 10-30% of EtOAc in petroleum ether as eluent to afford tert-butyl 4-(4-hydroxyphenyl)piperidine-1-carboxylate as an off-white solid (10.00 g, 85.6%). LCMS (ESI) m/z 278.2, [M+H]⁺.

### Step 5: tert-butyl 4-(4-(benzyloxy)phenyl)piperidine-1-carboxylate

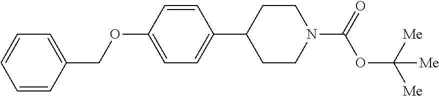 [SMILES: CC(C)(C)OC(=O)N1CCC(c2ccc(OCc3ccccc3)cc2)CC1]

To a solution of tert-butyl 4-(4-hydroxyphenyl)piperidine-1-carboxylate (11.20 g; 40.380 mmol; 1.00 eq.) and K₂CO₃ (11.2.0 g; 81.039 mmol; 2.01 eq.) in DMF (100 mL) was added (bromomethyl)benzene (10.40 g; 60.806 mmol, 1.51 eq.) at 0° C. The resulting solution was stirred at room temperature for 1 hour. The desired product was detected via LCMS. The resulting mixture was diluted with brine (200 mL) and extracted with EtOAc (3×100 mL). The combined organic layers were washed with brine (2×100 and dried over anhydrous Na₂SO₄, filtered, and concentrated under reduced pressure. The residue was purified by flash chromatography on silica gel column using 10-30% of EtOAc in petroleum ether as eluent to afford tort-butyl 4-(4-(benzyloxy)phenyl)piperidine-1-carboxylate as a colorless oil (14.00 g, 94.3%). LCMS (ESI) m/z 368.2, [M+H]⁺.

### Step 6: 4-(4-(benzyloxy)phenyl)piperidine hydrochloride

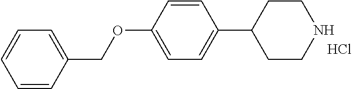 [SMILES: c1ccc(COc2ccc(C3CCNCC3)cc2)cc1 ; Cl]

To a solution of tert-butyl 4-(4-(benzyloxy)phenyl)piperidine-1-carboxylate (16.70 g; 45.444 mmol, 1.00 eq.) in CH₂Cl₂ (200 mL) was added HCl (gas) (4 M in 1,4-dioxane, 20 mL). The mixture was stirred at room temperature for 1 hour. The desired product was detected via LCMS. The precipitated product were collected by filtration and washed with CH₂Cl₂ (20 mL) just once to afford 4-(4-(benzyloxy)phenyl)piperidine hydrochloride as a white solid (13.00 g, 94.1%). LCMS (ESI) m/z 268.2, [M+H]⁺.

### Step 7: 3-(4-(4-(benzyloxy)phenyl)piperidin-1-yl)-4-tert-butyldiphenylsilyl)oxy)tetrahydrofuran-3-carbonitrile

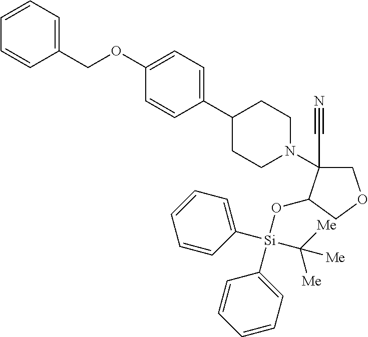 [SMILES: CC(C)(C)[Si](OC1COCC1(C#N)N1CCC(c2ccc(OCc3ccccc3)cc2)CC1)(c1ccccc1)c1ccccc1]

A solution of 4-(4-(benzyloxy)phenyl)piperidine hydrochloride (10.00 g; 32.913 mmol; 1 eq.) and AcOH (2.20 g; 36.635 mmol; 1.11 eq.) in dichloroethane (200 mL) was stirred at 50° C. for 1 hour. To the above mixture were added 4-((cert-butyldiphenylsilyl)oxy)dihydrofuran-3(2H)-one (17.0 g; 49.927 mmol; 1.52 eq.) and DIPEA (13.0 g; 100.583 mmol; 3.06 eq.) at 50° C. After stirring at 50° C. for 1 hour, to the above mixture was added TMSCN (5.00 g; 50.399 mmol; 1.53 eq.) at 50° C. and stirred at 50° C. for 14 hours. The desired product was detected via LCMS. The mixture was allowed to cool down to room temperature. The reaction was diluted with saturated NaHCO₃ solution (200 mL) and extracted with CH₂Cl₂ (2×200 mL). The combined organic layers were washed with water (2×100 mL) and dried over anhydrous Na₂SO₄. After filtration, the filtrate was concentrated under reduced pressure to afford 3-(4-(4-(benzyloxy)phenyl)piperidin-1-yl)-4-((tert-butyldiphenylsilyl)oxy)tetrahydrofuran-3-carbonitrile as a yellow oil (33.00 g, crude). The crude product was used in the next step directly without further purification. LCMS (ESI) m/z 617.3, [M+H]⁺.

### Step 8: 4-(4-(benzyloxy)phenyl)-1-(4-((tert-butyldiphenylsilyl)oxy)-3-methyltetrahydrofuran-3-yl)piperidine racemate)

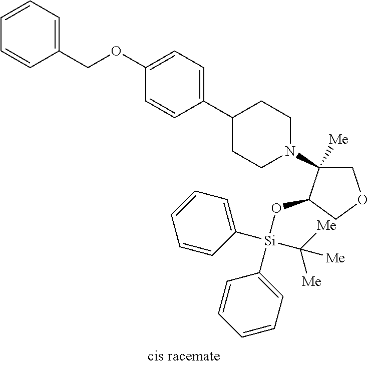 [SMILES: CC(C)(C)[Si](O[C@@H]1COC[C@]1(C)N1CCC(c2ccc(OCc3ccccc3)cc2)CC1)(c1ccccc1)c1ccccc1]

To a stirred solution of 3-(4-(4-(benzyloxy)phenyl)piperidin-1-yl)-4-((tert-butyldiphenylsilyl)oxy)tetrahydrofuran-3-carbonitrile (33.00 g; 53.495 mmol, 1 eq.) in THF (300 mL) was added methylmagnesium bromide (1 M in THF, 160.0 mL) drop-wise at 0° C. The resulting solution was stirred at 50° C. for 3 hours under nitrogen atmosphere. The desired product was detected via LCMS. The mixture was allowed to cool down to room temperature. Most of the solvent was removed under reduced pressure, the mixture was diluted with EtOAc (200 mL) and quenched with brine (100 mL) at 0° C. After separating the two phases, the aqueous phase was extracted with EtOAc (2×200 mL). The combined organic layers were washed with brine (2×100 mL) and dried over anhydrous Na₂SO₄, filtered, and concentrated under reduced pressure. The residue was purified by flash chromatography on silica gel column using 10-30% of EtOAc in petroleum ether as eluent to afford 4-(4-(benzyloxy)phenyl)-1-(4-((tert-butyldiphenylsilyl)oxy)-3-methyltetrahydrofuran-3-yl)piperidine (cis racemate) as a colorless oil (7.00 g, 21.6%). LCMS (ESI) m/z 606.3, [M+H]⁺.

### Step 9: 4-(1-(4-((tert-butyldiphenylsilyl)oxy)-3-methyltetrahydrofuran-3-yl)piperidin-4-yl)phenol (cis racemate)

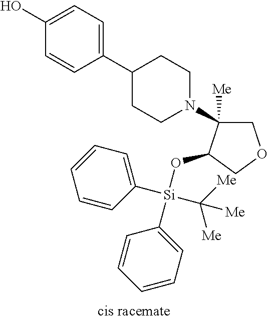 [SMILES: CC(C)(C)[Si](O[C@@H]1COC[C@]1(C)N1CCC(c2ccc(O)cc2)CC1)(c1ccccc1)c1ccccc1]

To a solution of 4-(4-(benzyloxy)phenyl)-1-(4-((tert-butyldiphenylsilyl)oxy)-3-methyltetrahydrofuran-3-yl)piperidine (7.00 g; 11.553 mmol; 1.00 eq.) in MeOH (100 mL) was added 10% Pd/C (0.70 g; 10% w/w) under nitrogen atmosphere. The mixture was hydrogenated at room temperature for 3 hours under hydrogen atmosphere (2 atm). After filtration, the filtrate was concentrated under reduced pressure to afford 4-(1-(4-((tert-butyldiphenylsilyl)oxy)-3-methyltetrahydrofuran-3-yl)piperidin-4-yl)phenol (cis racemate) as a white solid (5.00 g, 83.9%). LCMS (ESI) m/z 516.3, [M+H]⁺.

### Step 10: 4-(1-(4-tert-butyldiphenylsilyl)oxy)-3-methyltetrahydrofuran-3-yl)piperidin-4-yl)-2-nitrophenol (cis racemate)

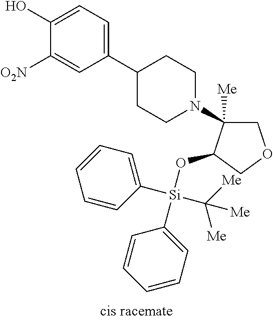 [SMILES: CC(C)(C)[Si](O[C@@H]1COC[C@]1(C)N1CCC(c2ccc(O)c([N+](=O)[O-])c2)CC1)(c1ccccc1)c1ccccc1]

A solution of 4-(1-(4-((tert-butyldiphenylsilyl)oxy)-3-methyltetrahydrofuran-3-yl)piperidin-4-yl)phenol (cis racemate) (5.00 g; 9,694 mmol; 1.00 eq.) and urea nitrate (2.50 g; 20.314 mmol, 2.10 eq.) in a mixture solvent of acetonitrile/water (20:1, 168 mL) was stirred at 80° C. for 1 hour under nitrogen atmosphere. The desired product was detected via LCMS. The mixture was allowed to cool down to room temperature. The acetonitrile was removed under reduced pressure, whereupon the precipitate product was collected by filtration and washed with water (2×20 mL) to afford the crude product. The crude product was purified by flash chromatography on silica gel column using 1-8% of MeOH in CH₂Cl₂ as eluent to afford 4-(1-(4-((tert-butyldiphenylsilyl)oxy)-3-methyltetrahydrofuran-3-yl)piperidin-4-yl)-2-nitrophenol (cis racemate) as a yellow solid (2.50 g, 45.9%). LCMS (ESI) m/z 561.3, [M+H]⁺.

### Step 11: 2-amino-4-(1-(4-((tert-butyldiphenylsilyl)oxy)-3-methyltetrahydrofuran-3-yl)piperidin-4-yl)phenol (cis racemate)

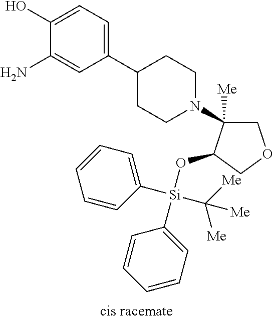 [SMILES: CC(C)(C)[Si](O[C@@H]1COC[C@]1(C)N1CCC(c2ccc(O)c(N)c2)CC1)(c1ccccc1)c1ccccc1]

To a solution of 4-(1-(4-((tert-butyldiphenylsilyl)oxy)-3-methyltetrahydrofuran-3-yl)piperidin-4-yl)-2-nitrophenol (cis racemate) (800.0 mg; 1.427 mmol, 1.00 eq.) in THF (50 mL) was added 10% Pd/C (0.20 g; 25% w/w) under nitrogen atmosphere. The mixture was hydrogenated at room temperature for 3 hours under hydrogen atmosphere (2 atm). After filtration, the filtrate was concentrated under reduced pressure to afford 2-amino-4-(1-(4-((tert-butyldiphenylsilyloxy)-3-methyltetrahydrofuran-3-yl)piperidin-4-yl)phenol (cis racemate) as an off-white solid (750.0 mg, crude). LCMS (ESI) m/z 531.3, [M+H]⁺.

### Step 12: N-(5-(5-(1-(4-hydroxy-3-methyltetrahydrofuran-3-yl)piperidin-4-yl)benzo[d]oxazol-2-yl)-8-(methylamino)-2,7-naphthyridin-3-yl)-cyclopropanecarboxamide (cis racemate)

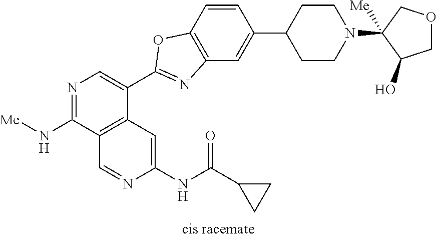 [SMILES: CNc1ncc(-c2nc3cc(C4CCN([C@@]5(C)COC[C@H]5O)CC4)ccc3o2)c2cc(NC(=O)C3CC3)ncc12]

N-(5-(5-(1-(4-hydroxy-3-methyltetrahydrofuran-3-yl)piperidin-4-yl)benzo[d]oxazol-2-yl)-8-(methylamino)-2,7-naphthyridin-3-yl)cyclopropanecarboxamide (cis racemate) was synthesized using a similar procedure that was previously described in Example 210, by using N-(5-formyl-8-(methylamino)-2,7-naphthyridin-3-yl)cyclopropanecarboxamide and 2-amino-4-(1-(4-((tert-butyldiphenylsilyl)oxy)-3-methy tetrahydrofuran-3-yl)piperidin-4-yl)phenol (cis racemate) as the starting material. LCMS (ESI) m/z 543.3, [M+H]⁺. ¹⁻H NMR (400 MHz, DMSO-d₆) δ 11.04 (s, 1H), 9.69 (s, 1H), 9.44 (s, 1H), 8.90 (s, 1H), 8.67-8.59 (m, 1H), 7.66-7.60 (m, 2H), 7.28 (dd, J=8.4, 1.6 Hz, 1H), 4.42-4.38 (m, 1H), 3.99-3.91 (m, 1H), 3.82-3.68 (m, 2H), 3.66-3.52 (m, 2H), 3.09 (d, J=4.4 HZ, 3H), 2.86-2.79 (m, 1H), 2.73-2.64 (m, 1H), 2.52-2.45 (m, 2H), 2.40-2.35 (m, 1H), 2.12-2.08 (m, 1H), 1.88-1.81 (m, 4H), 1.04 (s, 3H), 0.96-0.81 (m, 4H).

### Examples 261 and 262: Synthesis of N-(5-(5-(1-((3S,4S)-4-hydroxy-3-methyltetrahydrofuran-3-3/1)piperidin-4-yl)benzo[d]oxazol-2-yl)-8-(methylamino)-2,7-naphthyridin-3-yl)cyclopropanecarboxamide (Example 261) and N-(5-(5-(1-((3R,4R)-4-hydroxy-3-methyltetrahydrofuran-3-yl)piperidin-4-yl)benzo[d]oxazol-2-yl)-8-(methylamino)-2,7-naphthyridin-3-yl)cyclopropanecarboxamide (Example 262)

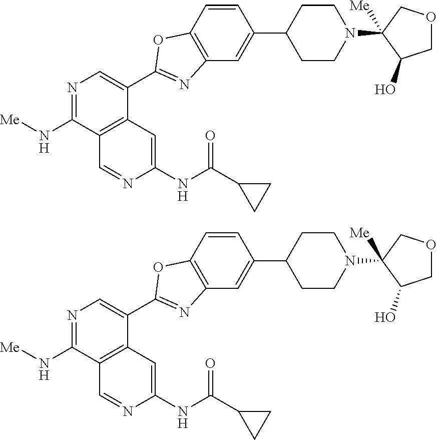

N-(5-(5-(1-(4-hydroxy-3-methyltetrahydrofuran-3-yl)piperidin-4-yl)benzo[d]oxazol-2-yl)-8-(methylamino)-2,7-naphthyridin-3-yl)cyclopropanecarboxamide (cis racemate) was separated by Prep-Chiral-HPLC (Column: CHIRALPAK IF, 2×25 cm, 5 μm; Mobile Phase A: Hex (0.5% 2 M NH₃-MeOH), Mobile Phase B: EtOH CH₂Cl₂=1:1; Flow rate: 20 mL/min; Gradient: 50% B to 50% B in 29 min) to afford N-(5-(5-(1-((3S,4S)-4-hydroxy-3-methyltetrahydrofuran-3-yl)piperidin-4-yl)benzo[d]oxazol-2-yl)-8-(methylamino)-2,7-naphthyridin-3-yl)cyclopropanecarboxamide (Example 261, the faster peak) and N-(5-(5-(1-(3R,4R)-4-hydroxy-3-methyltetrahydrofuran-3-yl)piperidin-4-yl)benzo[d]oxazol-2-yl)-8-(methylamino)-2,7-naphthyridin-3-yl)cyclopropanecarboxamide (Example 262, the slower peak) as yellow solids. The two configurations are arbitrarily assigned. LCMS (ESI) m/z 543.3, [M+H]⁺. HNMR for example 261, ¹H NMR (400 MHz, DMSO-d₆) δ 11.04 (s, 1H), 9.69 (s, 1H), 9.44 (s, 1H), 8.90 (s, 1H), 8.67-8.59 (m, 1H), 7.66-7.60 (m, 2H), 7.28 (dd, J=8.4, 1.6 Hz, 1H), 4.42-4.38 (m, 1H), 3.99-3.91 (m, 1H), 3.82-3.68 (m, 2H), 3.66-3.52 (m, 2H), 3.09 (d, 4.4 Hz, 3H), 2.86-2.79 (m, 1H), 2.73-2.64 (m, 1H), 2.52-2.45 (m, 2H), 2.40-2.35 (m, 1H), 2.12-2.08 (m, 1H), 1.88-1.81 (m, 4H), 1.04 (s, 3H), 0.96-0.81 (m, 4H).

HNMR for Example 262: 11.04 (s, 1H), 9.69 (s, 1H), 9.44 (s, 1H), 8.90 (s, 1H), 8.67-8.59 (m, 1H), 7.66-7.60 (m, 2H), 7.28 (dd, J=8.4, 1.6 Hz, 1H), 4.42-′4.38 (m, 1H), 3.99-3.91 (m, 1H), 3.82-3.68 (m, 2H), 3.66-3.52 (m, 2H), 3.09 (d, J=4.4 Hz, 3H), 2.86-2.79 (m, 1H), 2.73-2.64 (m, 1H), 2.52-2.45 (m, 2H), 2.40-2.35 (m, 1H), 2.12-2.08 (m, 1H), 1.88-1.81 (m, 4H), 1.04 (s, 3H), 0.96-0.81 (m, 4H).

### Example 263: Synthesis of N-(5-(7-(2-(methoxymethyl)azetidin-1-yl)benzo[d]oxazol-2-yl)-8-(methylamino)-2,7-naphthyridin-3-yl)cyclopropanecarboxamide

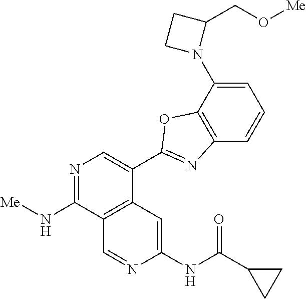 [SMILES: CNc1ncc(-c2nc3cccc(N4CCC4COC)c3o2)c2cc(NC(=O)C3CC3)ncc12]

### Step 1: N-(5-(7-bromobenzo[d]oxazol-2-yl)-8-(methylamino)-2,7-naphthyridin-3-yl)cyclopropanecarboxamide

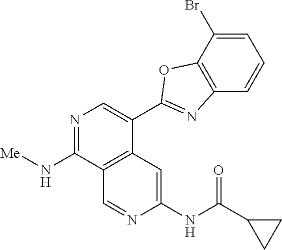 [SMILES: CNc1ncc(-c2nc3cccc(Br)c3o2)c2cc(NC(=O)C3CC3)ncc12]

A solution of 2-amino-6-bromophenol (139.1 mg; 0.740 mmol; 1.00 eq.) and N-(5-formyl-8-(methylamino)-2,7-naphthyridin-3-yl)cyclopropanecarboxamide (200.0 mg; 0.740 mmol; 1.00 eq.) in a mixture solvent of toluene/DMSO (5:1, 12.5 mL) was stirred at 110° C. for 36 hours. The mixture was concentrated under vacuum. The residue was dissolved by CH₂Cl₂ (12.5 mL), to this mixture was added DDQ (249.7 mg; 0.814 mmol; 1.10 eq.). The resulting solution was stirred at room temperature for 2 hours. The solvent was concentrated under reduced pressure. The residue was purified by flash chromatography on pre-packed C18 column using 30-100% of MeCN in water (10 mmol/L NH₄HCO₃) as eluent to provide N-(5-(7-bromobenzo[d]oxazol-2-yl)-8-(methylamino)-2,7-naphthyridin-3-yl)cyclopropanecarboxamide as a brown solid (200.0 mg, 61.6%). LCMS (ER) m/z 438.0, [M+H]⁺.

### Step 2: 11%-(5-(7-(2-(methoxymethyl)azetidin-1-yl)benzo[d]oxazol-2-yl)-8-(methylamino)-2,7-naphthyridin-3-yl)cyclopropanecarboxamide

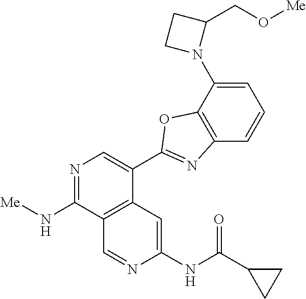 [SMILES: CNc1ncc(-c2nc3cccc(N4CCC4COC)c3o2)c2cc(NC(=O)C3CC3)ncc12]

A solution of N-(5-(7-bromobenzo[d]oxazol-2-yl)-8-(methyl amino)-2,7-naphthyridin-3-yl)cyclopropanecarboxamide (29.0 mg; 0.066 mmol; 1.00 eq.), 2-(methoxymethyl)azetidine hydrochloride (27.2 mg; 0.198 mmol; 3.00 eq.), Pd-PEPPSI-IHeptCl 3-chloropyridine (6.5 mg; 0.007 mmol; 0.10 eq.) and Cs₂CO₃ (43.1 mg; 0.132 mmol; 2.00 eq.) in 1,4-dioxane (1.5 ML) was stirred at 100° C. for 4 hours under nitrogen atmosphere. The mixture was concentrated under reduced pressure. The residue was purified by flash chromatography on silica gel column using 10-30% of MeOH in CH₂Cl₂ as eluent to provide a crude product. The crude product was purified by reverse phase preparative HPLC (Column: XBridge Prep OBD C18 Column, 30×150 mm, 5 μm, waters; gradient elution of 46-54% MeCN in water over a 8 min period, where water contains mmol/L NH₄HCO₃, flow rate: 60 mL/min, detector UV wavelength: 254 nm) to afford N-(5-(7-(2-(methoxymethyl)azetidin-1-yl)benzo[d]oxazol-2-yl)-8-(methylamino)-2,7-naphthyridin-3-yl)cyclopropanecarboxamide as a yellow solid (18.3 mg, 59.2%). LCMS (ESI) m/z 459.2, [M+H]⁺. ¹H NMR (400 MHz, DMSO-d₆) δ 11.03 (s, 1.14), 9.55 (s, 1H), 9.44 (s, 1H), 8.87 (s, 1H), 8.67-8.48 (m, 1H), 7.29-7.11 (m, 1H), 7.11-6.89 (m, 1H), 6.59 (d, J=8.0 Hz, 1H), 4.54-4.40 (m, 1H), 4.31-4.19 (m, 1H), 4.02-3.88 (m, 1H), 3.79-3.63 (m, 2H), 3.34 (s, 3H), 3.09 (d, J=4.4 Hz, 3H), 2.42-2.34 (m, 1H), 2.32-2.16 (m, 1H), 2.12-2.01 (m, 1R), 0.98-0.75 (m, 4H).

### Example 264: Synthesis of N-(5-(7-(3-(methoxymethyl)azetidin-1-yl)benzo[d]oxazol-2-yl)-8-(methylamino)-2,7-naphthyridin-3-yl)cyclopropanecarboxamide

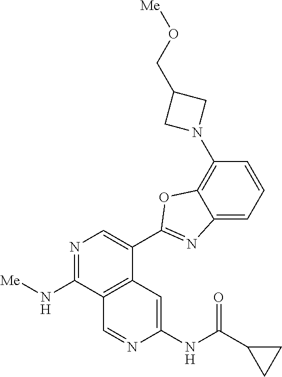 [SMILES: CNc1ncc(-c2nc3cccc(N4CC(COC)C4)c3o2)c2cc(NC(=O)C3CC3)ncc12]

N-(5-(7-(3-(methoxymethyl)azetidin-1-yl)benzo[d]oxazol-2-yl-8-(methylamino)-2,7-naphthyridin-3-yl)cyclopropanecarboxamide was synthesized using a similar procedure that was previously described in Example 263, by using N-(5-(7-bromobenzo[d]oxazol-2-yl)-8-(methylamino)-2,7-naphthyridin-3-yl)cyclopropanecarboxamide and 3-(methoxymethyl)azetidine hydrochloride as the starting material. LCMS (ESI) m/z 459.2, [M+H]⁺. ¹H NMR (400 MHz, DMSO-d₆) δ 11.04 (s, 1H), 9.51 (s, 1H), 9.44 (s, 1H), 8.89 (s, 1H), 8.75-8.40 (m, 1H), 7.25-7.10 (m, 1H), 7.05 (dd, J=8.0, 1.2 Hz, 1H), 6.36 (d, J=8.0 Hz, 1H), 4.33-4.11 (m, 2H), 3.99-3.81 (m, 2H), 3.60 (d, J=6.4 Hz, 2H), 3.31 (s, 3H), 3.09 (d, J=4.4 Hz, 3H), 3.04-2.93 (m, 1H), 2.19-2.00 (m, 1H), 1.00-0.76 (m, 4H).

### Example 265: Synthesis of N-(5-(7-(2-(hydroxymethyl)azetidin-t-yl)benzo[d]oxazol-2-yl)-8-(methylamino)-2,7-naphthyridin-3-yl)cyclopropanecarboxamide

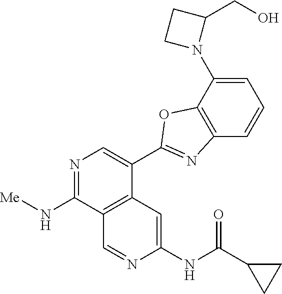 [SMILES: CNc1ncc(-c2nc3cccc(N4CCC4CO)c3o2)c2cc(NC(=O)C3CC3)ncc12]

N-(5-(7-bromobenzo[d]oxazol-2-yl)-8-(methylamino)-2,7-naphthyridin-3-yl)cyclopropanecarboxamide was synthesized using a similar procedure that was previously described in Example 263, by using N-(5-(7-bromobenzo[d]oxazol-2-yl)-8-(methylamino)-2,7-naphthyridin-3-yl)cyclopropanecarboxamide and (azetidin-2-yl)methanol hydrochloride as the starting material. LCMS (ESI) m/z 445.2, [M+H]⁺. ¹H NMR (400 MHz, DMSO-d₆) δ 11.05 (s, 1H), 9.59 (s, 1H), 9.44 (s, 1H), 8.88 (s, 1H), 8.67-8.60 (m, 1H), 7.20-7.11 (m, 1H), 7.05 (d, J=7.8 Hz, 1H), 6.60 (d, J=7.8 Hz, 1H), 4.93 (t, J=5.4 Hz, 1H), 4.43-4.34 (m, 1H), 4.28-4.18 (m, 1H), 3.99-3.89 (m, 1H), 3.81-3.75 (m, 2H), 3.09 (d, J=4.4 Hz, 3H), 2.41-2.22 (m, 2H), 2.15-2.04 (m, 1H), 0.94-0.81 (m, 4H).

### Example 266: Synthesis of N-(5-(7-(cyanomethyl)benzo[d]oxazol-2-yl)-8-(methylamino)-2,7-naphthyridin-3-yl)cyclopropanecarboxamide

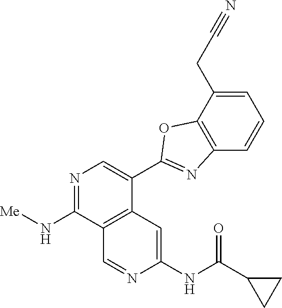 [SMILES: CNc1ncc(-c2nc3cccc(CC#N)c3o2)c2cc(NC(=O)C3CC3)ncc12]

To a solution of N-(5-(7-bromobenzo[d]oxazol-2-yl)-8-(methylamino)-2,7-naphthyridin-3-yl)cyclopropanecarboxamide (Example 263, step 1) (50.0 mg; 0.114 mmol; 1.00 eq.), 4-(4,4,5,5-tetramethyl-1,3,2-dioxaborolan-2-yl)isoxazole (26.7 mg; 0.137 mmol; 1.21 eq.) in DMSO (2 mL) were added Pd(dppf)Cl₂ (9.0 mg; 0.012 mmol; 0.11 eq.) and KF (9.9 mg; 0.171 mmol; 1.50 eq.) under nitrogen atmosphere. The resulting solution was stirred at 140° C. for 0.5 h. The desired product was detected via LCMS. The resulting mixture was diluted with EtOAc (20 mL) and washed by brine (3×5 mL). The organic phase was concentrated under reduced pressure. The residue was purified by flash chromatography on silica gel column using 0-10% of MeOH in CH₂Cl₂ as eluent to provide a crude product. The crude product was purified by flash chromatography on pre-packed C18 column using 20-60% of acetonitrile in water (10 mmol/L NH₄HCO₃) as eluent to provide N-(5-(7-(cyanomethyl)benzo[d]oxazol-2-yl)-8-(methylamino)-2,7-naphthyridin-3-yl)cyclopropanecarboxamide as a yellow solid (7.2 mg, 15.8%). LCMS (ESI) m/z 399.1, [M+H]⁺. ¹H NMR (400 MHz, DMSO-d₆) δ 11.08 (s, 1H), 9.74 (s, 1H), 9.45 (s, 1H), 9.03 (s, 1H), 8.75-8.67 (m, 1H), 7.74 (d, J=7.6 Hz, 1H), 7.46-7.34 (m, 2H), 4.44 (s, 2H), 3.10 (d, J=4.4 Hz, 3H), 2.16-2.06 (m, 1H), 0.97-0.83 (m, 4H).

### Example 267: Synthesis of N-(5-(7-(methoxymethyl)benzo[d]oxazol-2-yl)-8-(methylamino)-2,7-naphthyridin-3-yl)cyclopropanecarboxamide

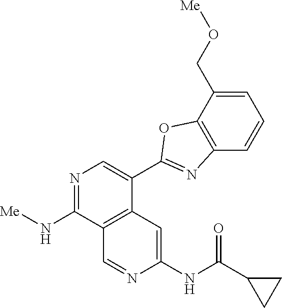 [SMILES: CNc1ncc(-c2nc3cccc(COC)c3o2)c2cc(NC(=O)C3CC3)ncc12]

To a solution of N-(5-(7-bromobenzo[d]oxazol-2-yl)-8-(methylamino)-2,7-naphthyridin-3-yl)cyclopropanecarboxamide (50.0 mg; 0.114 mmol; 1.00 eq.) in dioxane (3 mL) were added Pd(PPh₃)₄ (13.2 mg; 0.011 mmol; 0.10 eq.) and tributyl(methoxymethyl)stannane (115.0 mg; 0.343 mmol; 3.01 eq.) at room temperature. The reaction was stirred at 90° C. for 16 hours under nitrogen atmosphere. The desired product was detected via LCMS. The resulting mixture was concentrated under vacuum. The residue was purified by flash chromatography on silica gel column using 2-10% of MeOH in CH₂Cl, as eluent to provide the crude product. The crude product was purified by flash chromatography on pre-packed C18 column using 20-70% of MeCN in water (10 mmol/L NH₄HCO₃) to provide N-(5-(7-(methoxymethyl)benzo[d]oxazol-2-yl)-8-(methylamino)-2,7-naphthyridin-3-yl)cyclopropanecarboxamide as a yellow solid (21.3 mg, 46.3%). LCMS (ESI) m/z 404.2, [M+H]⁺. ¹H NMR (400 MHz, DMSO-d₆) δ 11.07 (s, 1H), 9.68 (s, 1H), 9.44 (s, 1H), 8.99 (s, 1H), 8.70-8.63 (m, 1H), 7.69 (dd, J=6.8, 2.0 Hz, 1H), 7.42-7.32 (m, 2H), 4.84 (s, 2H), 3.41 (s, 3H), 3.10 (d, 4.4 HZ, 3H), 2.16-2.07 (m, 1H), 0.96-0.90 (m, 2H), 0.89-0.84 (m, 2H).

### Example 268: Synthesis of (Z)—N-(5-(7-(2-ethoxyvinyl)benzo[d]oxazol-2-yl)-8-(methylamino 2,7-naphthyridin-3-yl)cyclopropanecarboxamide

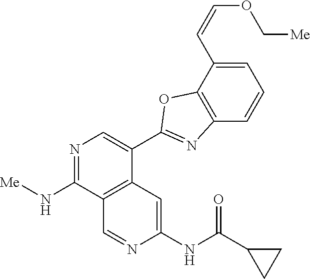 [SMILES: CCO/C=C\c1cccc2nc(-c3cnc(NC)c4cnc(NC(=O)C5CC5)cc34)oc12]

(Z)—N-(5-(7-(2-ethoxyvinyl)benzo[d]oxazol-2-yl)-8-(methylamino)-2,7-naphthyridin-3-yl)cyclopropanecarboxamide was synthesized using a similar procedure that was previously, described in Example 267 by using N-(5-(7-bromobenzo[d]oxazol-2-yl)-8-(methylamino)-2,7-naphthyridin-3-yl)cyclopropanecarboxamide and (Z)-tributyl(2-ethoxyvinyl)stannane as the starting material, LCMS (ESI) m/z 430.2, [M+H]⁺. ¹H NMR (400 MHz, DMSO-d₆) δ 11.06 (s, 1H), 9.71 (s, 1H), 9.44 (s, 1H), 9.02 (s, 1H), 8.71-8.58 (m, 1H), 7.92-7.88 (m, 1H), 7.53-7.50 (m, 1H), 7.35-7.27 (m, 1H), 6.65 (d, J=6.8 Hz, 1H), 5.87 (d, J=6.8 Hz, 1H), 4.17-4.09 (m, 2H), 3.10 (d, J=4.4 Hz, 3H), 2.16-2.05 (m, 1H), 1.33 (t, J=6.8 Hz, 3H), 0.99-0.79 (m, 4H).

### Example 269: Synthesis of N-(5-(7-(2-ethoxyethyl)benzo[d]oxazol-2-yl)-8-(methylamino)-2,7-naphthyridin-3-yl)cyclopropanecarboxamide

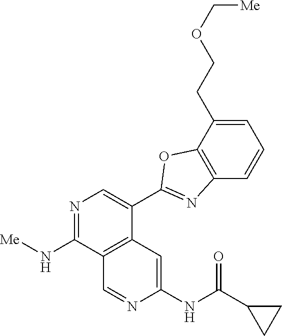 [SMILES: CCOCCc1cccc2nc(-c3cnc(NC)c4cnc(NC(=O)C5CC5)cc34)oc12]

To a solution of (Z)—N-(5-(7-(2-ethoxyvinyl)benzo[d]oxazol-2-yl)-8-(methylamino)-2,7-naphthyridin-3-yl)cyclopropanecarboxamide (Example 268) (25.0 mg; 0.058 mmol; 1.00 eq.) in MeOH (10 mL) was added 10% Pd/C (25.0 mg; 100% w/w) under nitrogen atmosphere. The mixture was hydrogenated for 14 hours at room temperature under hydrogen atmosphere (2 atm). After filtering through a celite pad, the filtrate was concentrated under reduced pressure. The residue was purified by flash chromatography on pre-packed C18 column using 20-60% of acetonitrile in water (10 mmol/L NH₄HCO₃) to provide N-(5-(7-(2-ethoxyethyl)benzo[d]oxazol-2-yl)-8-(methylamino)-2,7-naphthyridin-3-yl)cyclopropanecarboxamide as a brown yellow solid (17.4 mg, 69.2%). LCMS (ESI) m/z 432.2, [M+H]⁺. ¹H NMR (400 MHz, DMSO-d₆)d 11.06 (s, 1H), 9.64 (s, 1H), 9.46 (s, 1H), 8.96 (s, 1H), 8.77-8.61 (m, 1H), 7.60 (d, J=7.6 Hz, 1H), 7.36-7.20 (m, 2H), 3.78 (t, 6.8 Hz, 2H), 3.49 (q, J=7.0 Hz, 2H), 3.21 (t, J=6.8 Hz, 2H), 3.10 (d, J=4.4 Hz, 3H), 2.16-2.05 (m, 1H), 1.09 (t, 1=7.0 Hz, 3H), 0.97-0.79 (m, 4H).

### Example 270: Synthesis of N-(5-(7-(2-cyanoethyl)benzo[d]oxazol-2-yl)-8-(methylamino)-2,7-naphthyridin-3-yl)cyclopropanecarboxamide

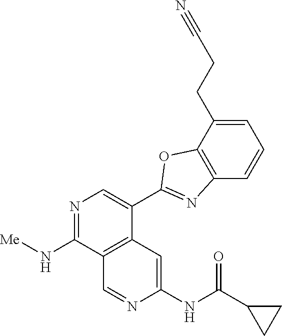 [SMILES: CNc1ncc(-c2nc3cccc(CCC#N)c3o2)c2cc(NC(=O)C3CC3)ncc12]

### Step 1: (E)-N-(5-(7-(2-cyanovinyl)benzo[d]oxazol-2-yl)-8-(methylamino)-2,7-naphthyridin-3-yl)cyclopropanecarboxamide

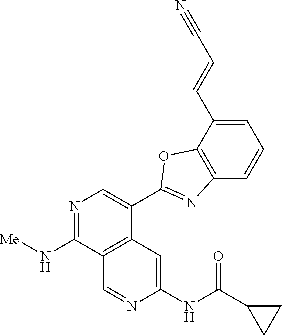 [SMILES: CNc1ncc(-c2nc3cccc(/C=C/C#N)c3o2)c2cc(NC(=O)C3CC3)ncc12]

To a solution of N-(5-(7-bromobenzo[d]oxazol-2-yl)-8-(methylamino)-2,7-naphthyridin-3-yl)cyclopropanecarboxamide (Example 263, step 1) (70.0 mg; 0.160 mmol; 1.00 eq.), Pd(PPh₃)₂Cl₂ (11.2 mg; 0.016 mmol; 0.10 eq.) and Na₂CO₃ (33.9 mg, 0.320 mmol; 2.00 eq.) in DMF (3 mL) was added acrylonitrile (84.7 mg; 1.596 mmol; 9.99 eq.) via syringe. The resulting solution was stirred at 120° C. overnight under nitrogen atmosphere. Upon completion, the resulting mixture was diluted with EtOAc (50 mL) and washed with brine (5×10 mL). The organic layer was dried with Na₂SO₄, filtered, and concentrated under vacuum. The residue was purified by flash chromatography on silica gel column using 0-10% of MeOH in CH₂Cl₂ as eluent to provide (E)-N-(5-(7-(2-cyanovinyl)benzo[d]oxazol-2-yl)-8-(methylamino)-2,7-naphthyridin-3-yl)cyclopropanecarboxamide as a yellow green solid (60.0 mg, 92.0%). LCMS (ER) m/z 411.1, [M+H]⁺.

### Step 2: N-(5-(7-(2-cyanoethyl)benzo(dioxazol-2-yl)-8-(methylamino)-2,7-naphthyridin-3-yl)cyclopropanecarboxamide

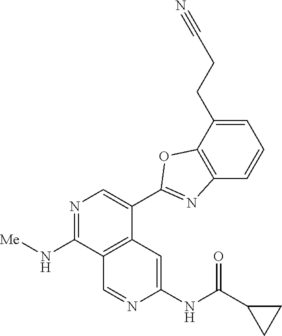 [SMILES: CNc1ncc(-c2nc3cccc(CCC#N)c3o2)c2cc(NC(=O)C3CC3)ncc12]

To a solution of (E)-N-(5-(7-(2-cyanovinyl)benzo[d]oxazol-2-yl)-8-(methylamino)-2,7-naphthyridin-3-yl)cyclopropanecarboxamide (50.0 mg; 0.122 mmol; 1.00 eq.) in MeOH (30 mL) was added 10% Pd/C (40.0 mg; 80% w/w) under nitrogen atmosphere. The mixture was hydrogenated at 40° C. for 6 hours under hydrogen atmosphere (2 atm). The desired product was detected via LCMS. The resulting mixture was filtered, the filter cake was washed with MeOH (30 mL). The filtrate was concentrated under reduced pressure. The residue was purified by reverse phase preparative HPLC (Prep-C18, 5 μM Xselect CSH F-Phenyl OBD column, 19×250 mm, waters; gradient elution of 32%-37% MeCN in water over a 8 min period, where water contains 0.1% formic acid, flow rate: 20 mL/min, detector UV wavelength: 254 nm) to provide N-(5-(7-(2-cyanoethyl)benzo[d]oxazol-2-yl)-8-(methylamino)-2,7-naphthyridin-3-yl)cyclopropanecarboxamide as a yellow solid (17.6 mg, 35.0%). LCMS (ESI) m/z 413.2, [M+H]⁺. ¹H NMR (400 MHz, DMSO-d₆) δ 11.32 (s, 1H), 9.97-9.83 (m, 1H), 9.69 (s, 1H), 9.65 (s, 1H), 8.75 (s, 1H), 7.74-7.67 (m, 1H), 7.42-7.36 (m, 2H), 3.31 (t, J=7.2 Hz, 2H), 3.19 (d, J=4.4 Hz, 3H), 3.05 (t, J=7.2 Hz, 2H), 2.15-2.09 (m, 1H), 0.97-0.87 (m, 4H).

### Example 271: Synthesis of N-(8-(methylamino)-5-(7-(oxazol-4-ylmethyl)benzo[d]oxazol-2-yl)-2,7-naphthyridin-3-yl)cyclopropanecarboxamide

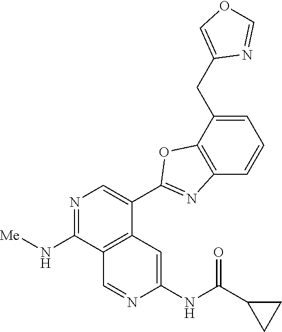 [SMILES: CNc1ncc(-c2nc3cccc(Cc4cocn4)c3o2)c2cc(NC(=O)C3CC3)ncc12]

### Step 1: N-(8-(methylamino)-5-(7-(4,4,5,5-tetramethyl-1,3,2-dioxaborolan-2-yl)benzo[d]oxazol-2-yl)-2,7-naphthyridin-3-yl)cyclopropanecarboxamide

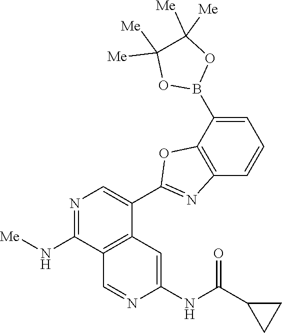 [SMILES: CNc1ncc(-c2nc3cccc(B4OC(C)(C)C(C)(C)O4)c3o2)c2cc(NC(=O)C3CC3)ncc12]

A solution of N-(5-(7-bromobenzo[d]oxazol-2-yl)-8-(methylamino)-2,7-naphthyridin-3-yl)cyclopropanecarboxamide (50.0 mg; 0.114 mmol; 1.00 eq.) and bis(pinacolato)diboron (290.0 mg; 1.142 mmol; 10.01 eq.), Pd(dppf)Cl₂·CH₂Cl₂ (9.3 mg; 0.011 mmol; 0.10 eq.), KOAc (22.4 mg; 0.228 mmol; 2.00 eq.) in dioxane (1.5 mL) was stirred at 100° C. for 3 hours under nitrogen atmosphere. The resulting mixture was concentrated under reduced pressure. The residue was dissolved in a mixture solvent of petroleum ether/EtOAc (10:1, 8 mL) to precipitate solids. The precipitated solids were collected by filtration and washed with a mixture solvent of petroleum ether/EtOAc (10:1, 8 mL). The solids were dried under reduced pressure to afford N-(8-(methylamino)-5-(7-(4,4,5,5-tetramethyl-1,3,2-dioxaborolan-2-yl)benzo[d]oxazol-2-yl)-2,7-naphthyridin-3-yl)cyclopropanecarboxamide as a brown solid (80.0 mg, crude). LCMS (ESI) m/z 486.2, [M+H]⁺.

### Step 2: 4-(bromomethyl)oxazole

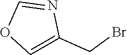

To a solution of oxazol-4-ylmethanol (100.0 mg; 1.009 mmol; 1.00 eq.) and EON (132.7 mg; 1.311 mmol; 1.30 eq.) in DMF (2 mL) was added methanesulfonyl methanesulfonate (457.0 mg; 2.623 mmol; 2.60 eq.) at 0° C. The reaction was stirred at 0° C. for 1 hour. To the above solution was added LiBr (245.4 mg; 2.826 mmol; 2.80 eq.) at 0° C. The reaction was stirred at 0° C. for 1 hour under nitrogen atmosphere. LC-MS indicated that the reaction was complete. The reaction was quenched with water and extracted with EtOAc (90 mL). The combined organic phase was washed with saturated brine (3×4 mL), dried with Na₂SO₄, filtered, and concentrated under reduced pressure. The residue was purified by flash chromatography on silica gel column using 2-25% of EtOAc in petroleum ether as eluent to provide 4-(bromomethyl)oxazole as yellow oil (100.0 mg, 61.5%). LCMS (ESI) m/z 161.9, [M+H]⁺.

### Step 3: N-(8-(methylamino)-5-(7-(oxazol-4-ylmethyl)benzo[d]oxazol-2-yl)-2,7-naphthyridin-3-yl)cyclopropanecarboxamide

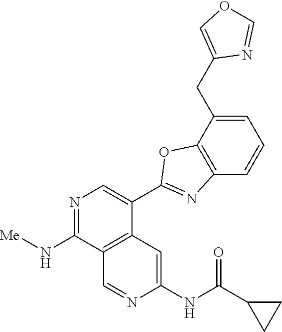 [SMILES: CNc1ncc(-c2nc3cccc(Cc4cocn4)c3o2)c2cc(NC(=O)C3CC3)ncc12]

To a solution of N-(8-(methylamino)-5-(7-(4,4,5,5-tetramethyl-1,3,2-dioxaborolan-2-yl) 71)benzo[d]oxazol-2-yl)-2,7-naphthyridin-3-yl)cyclopropanecarboxamide (80.0 mg; 0.165 mmol; 1.00 eq.) in a mixture of dioxane/water (5:1, 2.4 mL) was added XPhos Pd G₃ (14.2 mg; 0.017 mmol; 0.10 eq.), XPhos (8.3 mg; 0.017 mmol; 0.10 eq.), K₃PO₄ (105.6 mg; 0.495 mmol; 3.00 eq.) and 4-(bromomethyl)oxazole (265 mg; 0.164 mmol; 0.99 eq.) at room temperature. The reaction was stirred at 90° C. for 1 hour under nitrogen atmosphere. The resulting mixture was concentrated under vacuum. The residue was purified by flash chromatography on silica gel column using 2-10% of MeOH in CH₂Cl₂ as eluent to provide the crude product. The residue was purified by flash chromatography on pre-packed C18 column using 20-70% of MeCN in water (10 mmol/L NH₄HCO₃) to provide N-(8-(methylamino)-5-(7-(oxazol-4-ylmethyl)benzo[d]oxazol-2-yl)-2,7-naphthyridin-3-yl)cyclopropanecarboxamide as a yellow solid (9.0 mg, 12.3%). LCMS (ESI) m/z 441.2, [M+H]⁺. ¹H NMR (400 MHz, DMSO-d₆) δ 11.07 (s, 1H), 9.70 (s, 1H), 9.45 (s, 1H), 8.96 (s, 1H), 8.70-8.64 (m, 1H), 8.30 (s, 1H), 8.02. (s, 1H, 7.61 (d, J=7.6 Hz, 1H), 7.36-7.21 (m, 2H), 4.24 (s, 2H), 3.10 (d, J=4.0 Hz, 3H), 2.16-2.06 (m, 1H), 0.95-0.81 (m, 4H).

### Example 272: Synthesis of N-(5-(4-bromobenzo[d]oxazol-2-yl)-8-(methylamino)-2,7-naphthyridin-3-yl)cyclopropanecarboxamide

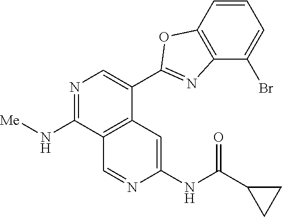 [SMILES: CNc1ncc(-c2nc3c(Br)cccc3o2)c2cc(NC(=O)C3CC3)ncc12]

A solution of 2-amino-3-bromophenol (139.1 mg; 0.740 mmol; 1.00 eq.) and N-(5-formyl-8-(methylamino)-2,7-naphthyridin-3-yl)cyclopropanecarboxamide (200.0 mg; 0.740 mmol; 1.00 eq.) in a mixture solvent of DMSO/xylene (1:5, 12 mL) was stirred at 110° C. for 48 hours. The desired product was detected via LCMS. The xylene was removed under vacuum. The residue was purified by flash chromatography on pre-packed C18 column using 20-70% of MeCN in water (10 mmol/L-NH₄HCO₃) to provide N-(5-(4-bromobenzo[d]oxazol-2-yl)-8-(methylamino)-2,7-naphthyridin-3-yl)cyclopropanecarboxamide as a brown yellow solid (4.2 mg, 1.2%). LCMS (ESI) m/z 438.0, [M+H]⁺. ¹H NMR (400 MHz, DMSO-d₆) δ 11.03 (s, 1H), 9.64 (s, 1H), 9.45 (s, 1H), 8.93 (s, 1H), 8.84-8.55 (m, 1H), 7.76 (d, J=8.0 Hz, 1H), 7.61 (d, J=8.0 Hz, 1H), 7.48-7.22 (m, 1H), 3.10 (d, J=4.4 Hz, 3H), 2.17-2.01 (m, 1H), 0.91-0.83 (m, 4H).

### Example 273: Synthesis of N-(5-(4-(cyanomethyl)benzo[d]oxazol-2-yl)-8-(methylamino)-2,7-naphthyridin-3-yl)cyclopropanecarboxamide

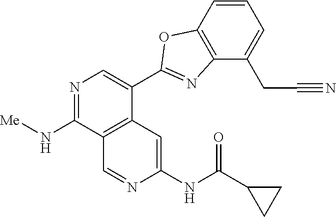 [SMILES: CNc1ncc(-c2nc3c(CC#N)cccc3o2)c2cc(NC(=O)C3CC3)ncc12]

### Step 1: N-(8-(methylamino)-5-(4-(4,4,5,5-tetramethyl-1,3,2-dioxaborolan-2-yl)benzo[d]oxazol-2-yl)-2,7-naphthyridin-3-yl)cyclopropanecarboxamide

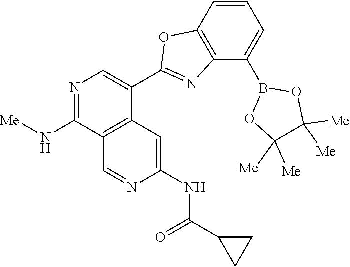 [SMILES: CNc1ncc(-c2nc3c(B4OC(C)(C)C(C)(C)O4)cccc3o2)c2cc(NC(=O)C3CC3)ncc12]

To a solution of N-(5-(4-bromobenzo[d]oxazol-2-yl)-8-(methylamino)-2,7-naphthyridin-3-yl)cyclopropanecarboxamide (Example 272) (50.0 mg; 0.114 mmol; 1.00 eq.) and Pin₂B₂(291.0 mg; 1.146 mmol; 10.05 eq.) in 1,4-dioxane (5 mL) were added KOAc (90.9 mg; 0.927 mmol; 8.13 eq.) and Pd(dppf)Cl₂·CH₂Cl₂ (8.9 mg; 0.011 mmol; 0.10 eq.) under nitrogen atmosphere. After stirring at 100° C. for 3 hours, the resulting mixture was concentrated under reduced pressure. The residue was slurred in a mixture of petroleum ether/EtOAc (10:1, 10 mL), whereupon the precipitated product was collected by filtration to afford N-(8-(methylamino)-5-(4-(4,4,5,5-tetramethyl-1,3,2-dioxaborolan-2-yl)benzo[d]oxazol-2-yl)-2,7-naphthyridin-3-yl)cyclopropanecarboxamide as a black solid (236.2 mg, crude). LCMS (ESI) m/z 486.2, [M+H]⁺.

### Step 2: N-(5-(4-(cyanomethyl)benzo[d]oxa ol-2-yl)-8-(methylamino)-2,7-naphthyridin-3-yl)cyclopropanecarboxamide

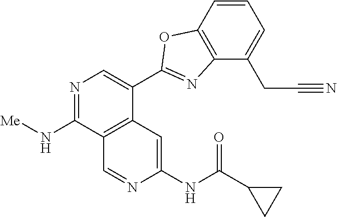 [SMILES: CNc1ncc(-c2nc3c(CC#N)cccc3o2)c2cc(NC(=O)C3CC3)ncc12]

A solution of N-(8-(methylamino)-5-(4-(4,4,5,5-tetramethyl-1,3,2-dioxaborolan-2-yl)benzo[d]oxazol-2-yl)-2,7-naphthyridin-3-yl)cyclopropanecarboxamide (200.0 mg; 0.412 mmol; 1.00 eq.) and 2-iodoacetonitrile (68.7 mg; 0.412 mmol; 1.00 eq.) in 1,4-dioxane/water (5:1, 12 mL). To the above solution were added K₃PO₄ (261.9 mg; 1.234 mmol; 3.00 eq.), XPhos Pd G₃ (34.7 mg; 0.041 mmol; 0.10 eq.) and XPhos (19.5 mg, 0.041 mmol, 0.10 eq.) under nitrogen atmosphere. The resulting solution was stirred at 90° C. for 1 hour. The resulting mixture was cooled to room temperature and concentrated under reduced pressure. The residue was purified by flash chromatography on silica gel column using 0-10% of MeOH in CH₂Cl₂ as eluent to provide N-(5-(4-(cyanomethyl)benzo[d]oxazol-2-yl)-8-(methylamino)-2,7-naphthyridin-3-yl)cyclopropanecarboxamide as a white solid (6.3 mg, 3.8%). LCMS (ESI) m/z 399.1, [M+H]⁺. ¹H NMR (400 MHz, DMSO-d₆) δ 11.07 (s, 1H), 9.96 (s, 1H); 9.45 (s, 1H), 8.94 (s, 1H), 8.75-8.68 (m, 1H), 7.78-7.69 (m, 1H), 7.48-7.38 (m, 2H), 4.38 (s, 2H), 3.10 (d, J 4.4 Hz, 3H), 2.15-2.06 (m, 1H), 0.96-0.90 (m, 2H), 0.90-0.84 (m, 2H).

### Example 274: Synthesis of N-(5-(6-(3-methoxyazetidin-1-yl)benzo[d]oxazol-2-yl)-8-(methylamino)-2,7-naphthyridin-3-yl)cyclopropanecarboxamide

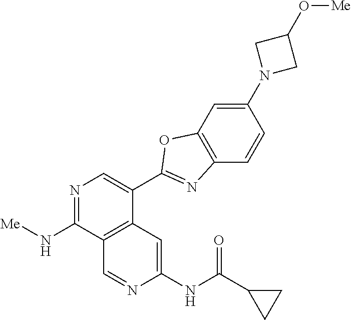 [SMILES: CNc1ncc(-c2nc3ccc(N4CC(OC)C4)cc3o2)c2cc(NC(=O)C3CC3)ncc12]

### Step 1: N-(5-(6-bromobenzo[d]oxazol-2-yl)-8-(methylamino)-2,7-naphthyridin-3-yl)cyclopropanecarboxamide

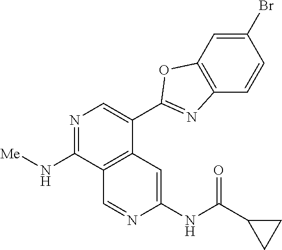 [SMILES: CNc1ncc(-c2nc3ccc(Br)cc3o2)c2cc(NC(=O)C3CC3)ncc12]

To a solution of 2-amino-5-bromophenol (138.5 mg; 0.737 mmol; 1.00 eq.) and N-(5-formyl-8-(methylamino)-2,7-naphthyridin-3-yl)cyclopropanecarboxamide (200.0 mg; 0.740 mmol; 1.00 eq.) in toluene (20 mL) was stirred at 110° C. for 16 hours. The resulting mixture was concentrated under vacuum and the residue was dissolved in CH₂Cl₂ (10 mL). To the above solution was added DDQ (185 mg; 0.815 mmol; 1.10 eq.). The reaction was stirred at room temperature for 2 hours under nitrogen atmosphere. The desired product was detected via LCMS. The resulting mixture was concentrated under vacuum. The residue was purified by flash chromatography on pre-packed C18 column using 20-70% of MeCN in water (10 mmol/L NH₄HCO₃) to provide N-(5-(6-bromobenzo[d]oxazol-2-yl)-8-(methylamino)-2,7-naphthyridin-3-yl)cyclopropanecarboxamide as a brown solid (157.0 mg, 48.4%). LCMS (ESI) m/z 438.0, [M+H]⁺.

### Step 2: N-(5-(6-(3-methoxyazetidin-1-yl)benzo[d]oxazol-2-yl)-8-(methylamino)-2,7-naphthyridin-3-yl)cyclopropanecarboxamide

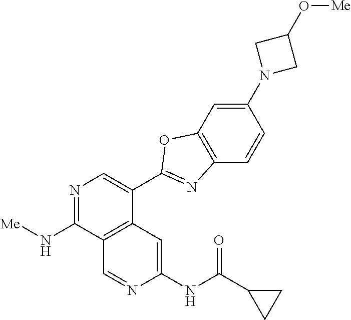 [SMILES: CNc1ncc(-c2nc3ccc(N4CC(OC)C4)cc3o2)c2cc(NC(=O)C3CC3)ncc12]

To a solution of N-(5-(6-bromobenzo[d]oxazol-2-yl)-8-(methylamino)-2,7-naphthyridin-3-yl)cyclopropanecarboxamide (50.0 mg; 0.114 mmol; LOU eq.) in 1,4-dioxane (3 mL) were added Pd-PEPPSI-IHeptCl 3-chloropyridine (11.1 mg; 0.011 mmol; 0.10 eq.), Cs₂CO₃ (186.5 mg; 0.572 mmol; 5.02 eq.) and 3-methoxyazetidine hydrochloride (42.4 mg; 0.343 mmol; 3.01 eq.) at room temperature. The reaction was stirred at 100° C. for 16 hours under nitrogen atmosphere. The desired product was detected via LCMS. The resulting mixture was concentrated under vacuum. The residue was purified by flash chromatography on silica gel column using 2-10% of MeOH in CH₂Cl₂ as eluent to provide the crude product. The crude product was purified by flash chromatography on pre-packed C18 column using 20-70% of MeOH in water (10 mmol/L NH₄HCO₃) to provide N-(5-(6-(3-methoxyazetidin-1-yl)benzo[d]oxazol-2-yl)-8-(methylamino)-2,7-naphthyridin-3-yl)cyclopropanecarboxamide as a yellow solid (28.4 mg, 56%). LCMS (ESI) m/z 445.2, [M+H]⁺. ¹H NMR (400 MHz, DMSO-d₆) δ 11.01 (s, 1H), 9.65 (s, 1H), 9.43 (s, 1H), 8.81 (s, 1H), 8.57-8.52 (m, 1H), 7.54 (d, J=8.6 Hz, 1H), 6.75 (d, J=2.0 Hz, 1H), 6.49 (d, 8.6 Hz, 1H), 4.39-4.32 (m, 1H), 4.16-4.08 (m, 2H), 3.72-3.64 (m, 2H), 3.27 (s, 3H), 3.08 (d, J=4.4 Hz, 3H), 2.16-2.03 (m, 1H), 0.94-0.89 (m, 2H), 0.88-0.84 (m, 2H).

### Example 275: Synthesis of (S)—N-(8-(methylamino)-5-(6-(2-methylmorpholino)benzo[d]oxazol-2-yl)-2,7-naphthyridin-3-yl)cyclopropanecarboxamide

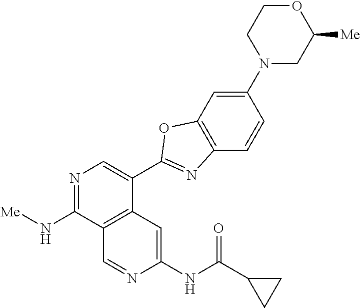 [SMILES: CNc1ncc(-c2nc3ccc(N4CCO[C@@H](C)C4)cc3o2)c2cc(NC(=O)C3CC3)ncc12]

To a solution of N-(5-(6-bromobenzo[d]oxazol-2-yl)-8-(methylamino)-2,7-naphthyridin-3-yl)cyclopropanecarboxamide (Example 274, step 1) (80.0 mg; 0.183 mmol; 1.00 eq.) in dioxane (3 mL) were added EPhos Pd G₄ (17.0 mg; 0.019 mmol; 0.10 eq.), EPhos (10.0 mg; 0.019 mmol; 0.10 eq.), Cs₂CO₃ (119.3 mg; 0.366 mmol; 2.01 eq.) and (S)-2-methylmorpholine (55.5 mg; 0.549 mmol; 3.01 eq.) at room temperature. The reaction was stirred at 100° C. for 16 hours under nitrogen atmosphere. The desired product was detected via LCMS. The resulting mixture was cooled to room temperature and concentrated under vacuum. The residue was purified by flash chromatography on silica gel column using 2-10% of MeOH, in CH₂Cl₂ as eluent to provide the crude product. The crude product was separated by Achiral-SFC (Column: GreenSep Naphthyl 3×25 cm, 5 μm; Mobile Phase A: CO₂, Mobile Phase B: MeOH (0.1% 2 M NH₃-MeOH); Flow rate: 75 mL/min; Gradient: isocratic 30% B; Wave Length: 254 nm; R_{T}(min): 7.75) to provide (S)—N-(8-(methylamino)-5-(6-(2-methylmorpholino)benzo[d]oxazol-2-yl)-2,7-naphthyridin-3-yl)cyclopropanecarboxamide as a yellow solid (25.5 mg, 30.4%). LCMS (ESI) m/z 459.2, [M+H]⁺. ¹H NMR (400 MHz, DMSO-d₆) δ 11.01 (s, 1H), 9.65 (s, 1H), 9.43 (s, 1H), 8.83 (s, 1H), 8.60-8.52 (m, 1H), 7.59 (d, 8.8 Hz, 1H), 7.27 (J=2.4 Hz, 1H), 7.05 (dd, J=8.8, 2.4 HZ, 1H), 3.98-3.90 (m, 1H), 3.75-3.62 (m, 3H), 3.41-3.38 (m, 1H), 3.08 (d, J=4.4 Hz, 3H), 2.79-2.68 (m, 1H), 2.47-2.37 (m, 1H), 2.15-2.05 (m, 1H), 1.19 (d, J=6.2 Hz, 314), 0.95-0.81 (m, 4H).

### Example 276: Synthesis of (R)—N-(8-(methylamino)-5-(6-(2-methylmorpholino)benzo[d]oxazol-2-yl)-2,7-naphthyridin-3-yl)cyclopropanecarboxamide

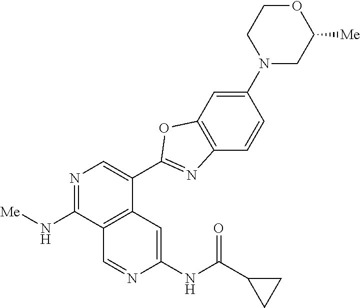 [SMILES: CNc1ncc(-c2nc3ccc(N4CCO[C@H](C)C4)cc3o2)c2cc(NC(=O)C3CC3)ncc12]

(R)—N-(8-(methylamino)-5-(6-(2-methylmorpholino)benzo[d]oxazol-2-yl)-2,7-naphthyridin-3-yl)cyclopropanecarboxamide was synthesized using a similar procedure that was previously described in Example 275, by using N-(5-(6-bromobenzo[d]oxazol-2-yl)-8-(methylamino)-2,7-naphthyridin-3-yl)cyclopropanecarboxamide and (R)-2-methylmorpholine as the starting material. LCMS (ESI) m/z 459.2, [M+H]⁺. ¹H NMR (400 MHz, DMSO-d₆) δ 11.01 (s, 1H), 9.65 (s, 1H), 9.43 (s, 1H), 8.83 (s, 1H), 8.60-8.52 (m, 1H), 7.59 (d, J=8.8 Hz, 1H), 7.27 (J=2.4 Hz, 1H), 7.05 (dd, J=8.8, 2.4 Hz, 1H), 3.98-3.90 (m, 1H), 3.75-3.62 (m, 3H), 3.41-3.38 (m, 1H), 3.08 (d, J=4.4 Hz, 3H), 2.79-2.68 (m, 1H), 2.47-2.37 (m, 1H), 2.15-2.05 (m, 1.19 (d, J=6.2 Hz, 314), 0.95-0.81 (m, 4H).

### Example 277: Synthesis of N-(5-(5-(azetidin-1-yl)benzo[d]oxazol-2-yl)-8-(methylamino)-2,7-naphthyridin-3-yl)cyclopropanecarboxamide

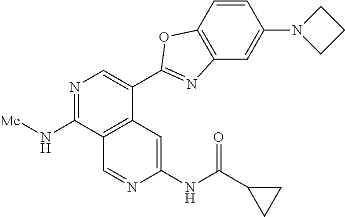 [SMILES: CNc1ncc(-c2nc3cc(N4CCC4)ccc3o2)c2cc(NC(=O)C3CC3)ncc12]

To a stirred solution of N-(5-(5-bromobenzo[d]oxazol-2-yl)-8-(methylamino)-2,7-naphthyridin-3-yl)cyclopropanecarboxamide (Example 209) (140.0 mg; 0.319 mmol; 1.00 eq.), Pd-PEPPSI-IHeptCl 3-chloropyridine (31.1 mg; 0.032 mmol; 0.10 eq.), Cs₂CO₃ (208.2 mg; 0.639 mmol, 2.00 eq.) in 1,4-dioxane (5 mL) was added azetidine (54.7 rag; 0.958 mmol; 3.00 eq.) under nitrogen atmosphere. The resulting solution was heated at 100° C. for 1 hour. The desired product was detected via LCMS. The mixture was concentrated under reduced pressure. The residue was purified by flash chromatography on silica gel column using 0-25% of MeOH in CH₂Cl₂ as eluent to provide the crude product. The crude product was purified by flash chromatography on pre-packed C18 column using 30-100% of MeCN in water (10 mmol/L NH₄HCO₃) as eluent to provide N-(5-(5-(azetidin-1-yl)benzo[d]oxazol-2-yl)-8-(methylamino)-2,7-naphthyridin-3-yl)cyclopropanecarboxamide as a yellow solid (85.8 mg, 64.8%). LCMS (ESI) m/z 415.2, [M+H]⁺. ¹H NMR (400 MHz, DMSO-d₆) δ 11.01 (s, 1H), 9.65 (s, 1H), 9.43 (s, 1H), 8.88 (s, 1H), 8.69-8.57 (m, 1.14), 7.52 (d, J=8.4 Hz, 1.14), 6.68 (d, J=2.0 Hz, 1H), 6.59-6.25 (m, 1H), 3.94-3.75 (m, 4H), 3.09 (d, J=4.4 Hz, 3H), 2.41-2.25 (m, 2H), 2.20-2.01 (m, 1H), 0.97-0.79 (m, 4H). Each compound in Table 12 below was prepared using a similar experimental procedure to prepare Example 277, using N-(5-(5-bromobenzo[d]oxazol-2-yl)-8-(methylamino)-2,7-naphthyridin-3-yl)cyclopropanecarboxamide as the common intermediate and appropriate amine.

TABLE 12

| Example | Structure and Name | MS and ¹H NMR |
|---|---|---|
| Example 278 | 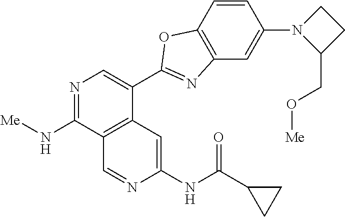 [SMILES: CNc1ncc(-c2nc3cc(N4CCC4COC)ccc3o2)c2cc(NC(=O)C3CC3)ncc12] | LCMS (ESI) m/z 459.2, [M + H]⁺. ¹H NMR (400 MHz, DMSO-d₆) δ 11.02 (s, 1H), 9.65 (s, 1H), 9.43 (s, 1H), 8.87 (s, 1H), 8.64-8.56 (m, 1H), 7.51 (d, J = 8.7 Hz, 1H), 6.89 (d, J = 2.3 Hz, 1H), 6.71-6.62 (m, 1H), 4.21- 4.10 (m, 1H), 3.99-3.90 (m, 1H), 3.73-3.64 (m, 1H), 3.63-3.49 (m, 2H), 3.38 (s, 3H), 3.09 (d, J = 4.4 Hz, 3H), 2.38-2.25 (m, 1H), 2.23-2.05 (m, 2H), 0.96-0.81 (m, 4H). |
| | N-(5-(5-(2- | |
| | (methoxymethyl)azetidin-1- | |
| | yl)benzo[d]oxazol-2-y1)-8- | |
| | (methylamino)-2,7-naphthyridin-3- | |
| | yl)cyclopropanecarboxamide | |
| Example 279 | 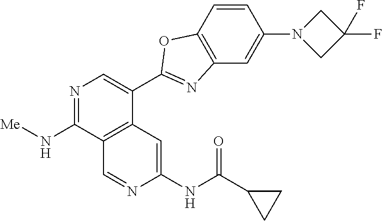 [SMILES: CNc1ncc(-c2nc3cc(N4CC(F)(F)C4)ccc3o2)c2cc(NC(=O)C3CC3)ncc12] | LCMS (ESI) m/z 451.2, [M + H]⁺. ¹H NMR (400 MHz, DMSO-d₆) δ 11.05 (s, 1H), 9.67 (s, 1H), 9.44 (s, 1H), 8.89 (s, 1H), 8.79-8.44 (m, 1H), 7.60 (d, J = 8.8 Hz, 1H), 6.91 (d, J = 2.4 Hz, 1H), 6.70-6.54 (m, 1H), 4.42- 4.26 (m, 4H), 3.09 (d, J = 4.4 Hz, 3H), 2.16-2.02 (m, 1H), 0.92-0.83 (m, 4H) |
| | N-(5-(5-(3,3-difluoroazetidin-1- | |
| | yl)benzo[d]oxazol-2-yl)-8- | |
| | (methylamino)-2,7-naphthyridin-3- | |
| | yl)cyclopropanecarboxamide | |
| Example 280 | 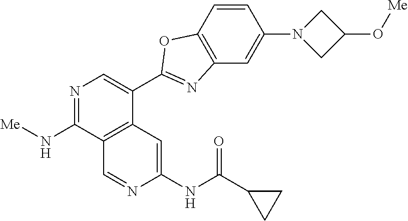 [SMILES: CNc1ncc(-c2nc3cc(N4CC(OC)C4)ccc3o2)c2cc(NC(=O)C3CC3)ncc12] | LCMS (ESI) m/z 445.2, [M + H]⁺. ¹H NMR (400 MHz, DMSO-d₆) δ 11.02 (s, 1H), 9.66 (s, 1H), 9.43 (s, 1H), 8.88 (s, 1H), 8.72-8.43 (m, 1H), 7.53 (d, J = 8.8 Hz, 1H), 6.72 (d, J = 2.4 Hz, 1H), 6.59-6.32 (m, 1H), 4.43- 4.25 (m, 1H), 4.20-4.02 (m, 2H), 3.80-3.52 (m, 2H), 3.27 (s, 3 H), 3.09 (d, J = 4.4 Hz, 3H), 2.20-2.00 (m, 1H), 0.99-0.79 (m, 4H). |
| | N-(5-(5-(3-methoxyazetidin-1- | |
| | yl)benzo[d]oxazol-2-y1)-8- | |
| | (methylamino)-2,7-naphthyridin-3- | |
| | yl)cyclopropanecarboxamide | |
| Example 281 | 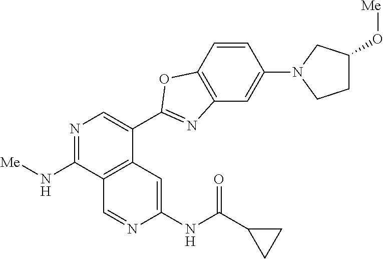 [SMILES: CNc1ncc(-c2nc3cc(N4CC[C@@H](OC)C4)ccc3o2)c2cc(NC(=O)C3CC3)ncc12] | LCMS (ESI) m/z 459.2, [M + H]⁺ ¹H NMR (400 MHz, DMSO-d₆) δ 11.03 (s, 1H), 9.66 (s, 1H), 9.43 (s, 1H), 8.88 (s, 1H), 8.63-8.57 (m, 1H), 7.52 (d, J = 8.8 Hz, 1H), 6.79 (d, J = 2.0 Hz, 1H), 6.62 (dd, J = 8.8, 2.0 Hz, 1H), 4.13-4.10 (m, 1H), 3.55-3.46 (m, 1H), 3.36 (s, 3H), 3.33-3.28 (m, 3H), 3.09 (d, J = 4.3 Hz, 3H), 2.19- 2.06 (m, 3H), 0.96-0.83 (m, 4H). |
| | (R)-N-(5-(5-(3-methoxypyrrolidin- | |
| | 1-yl)benzo[d]oxazol-2-y1)-8- | |
| | (methylamino)-2,7-naphthyridin-3- | |
| | yl)cyclopropanecarboxamide | |
| Example 282 | 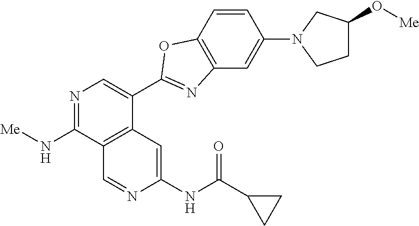 [SMILES: CNc1ncc(-c2nc3cc(N4CC[C@H](OC)C4)ccc3o2)c2cc(NC(=O)C3CC3)ncc12] | LCMS (ESI) m/z 459.2, [M + H]⁺. ¹H NMR (400 MHz, DMSO-d₆) δ 11.03 (s, 1H), 9.66 (s, 1H), 9.43 (s, 1H), 8.88 (s, 1H), 8.65-8.56 (m, 1H), 7.52 (d, J = 8.8 Hz, 1H), 6.79 (d, J = 2.4 Hz, 1H), 6.62 (dd, J = 8.8, 2.4 Hz, 1H), 4.16-4.08 (m, 1H), 3.55-3.45 (m, 2H), 3.31-3.26 (m, 5H), 3.08 (d, J = 4.4 Hz, 3H), 2.16-2.04 (m, 3H), 0.95-0.81 (m, 4H). |
| | (S)-N-(5-(5-(3-methoxypyrrolidin- | |
| | 1-yl)benzo[d]oxazol-2-y1)-8- | |
| | (methylamino)-2,7-naphthyridin-3- | |
| | yl)cyclopropanecarboxamide | |
| Example 283 | 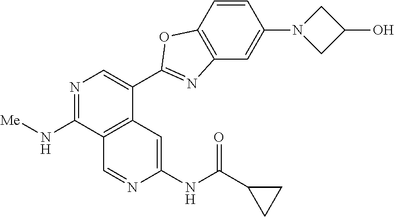 [SMILES: CNc1ncc(-c2nc3cc(N4CC(O)C4)ccc3o2)c2cc(NC(=O)C3CC3)ncc12] | LCMS (ESI) m/z 431.2, [M + H]⁺. ¹H NMR (400 MHz, DMSO-d₆) δ 11.02 (s, 1H), 9.65 (s, 1H), 9.43 (s, 1H), 8.87 (s, 1H), 8.65-8.57 (m, 1H), 7.52 (d, J = 8.8 Hz, 1H), 6.70 (d, J = 2.0 Hz, 1H), 6.53-6.44 (m, 1H), 5.61 (d, J = 6.4 Hz, 1H), 4.65-4.53 (m, 1H), 4.14 (t, J = 6.8 Hz, 2H), 3.58-3.51 (m, 2H), 3.09 (d, J = 4.4 Hz, 3H), 2.16-2.05 (m, 1H), 0.95-0.89 (m, 2H), 0.89-0.83 (m, 2H), |
| | N-(5-(5-(3-hydroxyazetidin-1- | |
| | yl)benzo[d]oxazol-2-y1)-8- | |
| | (methylamino)-2,7-naphthyridin-3- | |
| | yl)cyclopropanecarboxamide | |
| Example 284 | 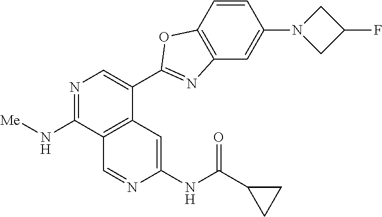 [SMILES: CNc1ncc(-c2nc3cc(N4CC(F)C4)ccc3o2)c2cc(NC(=O)C3CC3)ncc12] | LCMS (ESI) m/z 433.2, [M + H]⁺. ¹H NMR (400 MHz, DMSO-d₆) δ 11.02 (s, 1H), 9.65 (s, 1H), 9.43 (s, 1H), 8.88 (s, 1H), 8.69-8.45 (m, 1H), 7.56 (d, J = 8.8 Hz, 1H), 6.79 (d, J = 2.0 Hz, 1H), 6.61-6.39 (m, 1H), 5.66- 5.31 (m, 1H), 4.29-4.12 (m, 2H), 4.05-3.76 (m, 2H), 3.09 (d, J = 4.4 Hz, 3H), 2.19-2.03 (m, 1H), 0.97- 0.79 (m, 4H). |
| | N-(5-(5-(3-fluoroazetidin~1- | |
| | yl)benzo[d]oxazol-2-y1)-8- | |
| | (methylamino)-2,7-naphthyridin-3- | |
| | yl)cyclopropanecarboxamide | |
| Example 285 | 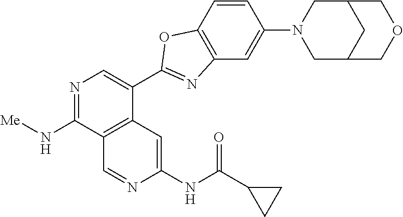 [SMILES: CNc1ncc(-c2nc3cc(N4CC5COCC(C5)C4)ccc3o2)c2cc(NC(=O)C3CC3)ncc12] | LCMS (ESI) m/z 485.2, [M + H]⁺. ¹H NMR (400 MHz, DMSO-d₆) δ 11.03 (s, 1H), 9.68 (s, 1H), 9.44 (s, 1H), 8.89 (s, 1H), 8.66-8.53 (m, 1H), 7.58- 7.50 (m, 1H), 7.06 (d, J = 2.4 Hz, 1H), 6.95 (d, J = 8.8 Hz, 1H), 3,90 (d, J = 10.8 Hz, 2H), 3.82 (d, J = 10.8 Hz, 2H), 3.68 (d, J = 10.8 Hz, 2H), 3.13- 3.05 (m, 5H), 2.15-2.04 (m, 1H), 2.00-1.87 (m, 3H), 1.83-1.72 (m, 1H), 0.95-0.87 (m, 2H), 0.87-0.81 (m, 2H). |
| | N-(5-(5-(3-oxa~7- | |
| | azabicyclo[3.3.1]nonan-7- | |
| | yl)benzo[d]oxazol-2-yl)-8- | |
| | (methylamino)-2,7-naphthyridin-3- | |
| | yl)cyclopropanecarboxamide | |
| Example 286 | 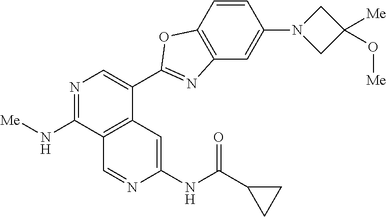 [SMILES: CNc1ncc(-c2nc3cc(N4CC(C)(OC)C4)ccc3o2)c2cc(NC(=O)C3CC3)ncc12] | LCMS (ESI) m/z 459.2, [M + H]⁺. ¹H NMR (300 MHz, DMSO-d₆) δ 11.01 (s, 1H), 9.65 (s, 1H), 9.43 (s, 1H), 8.87 (s, 1H), 8.68-8.57 (m, 1H), 7.53 (d, J = 8.4 Hz, 1H), 6.74 (d, J = 2.4 Hz, 1H), 6.55-6.48 (m, 1H), 3.76 (s, 4H), 3.22 (s, 3H), 3.09 (d, J = 4.4 Hz, 3H), 2.17-2.08 (m, 1H), 1.52 (s, 3H), 0.96-0.82 (m, 4H). |
| | N-(5-(5-(3-methoxy-3- | |
| | methylazetidin-1- | |
| | yl)benzo[d]oxazol-2-yl)-8- | |
| | (methylamino)-2,7-naphthyridin-3- | |
| | yl)cyclopropanecarboxamide | |
| Example 287 | 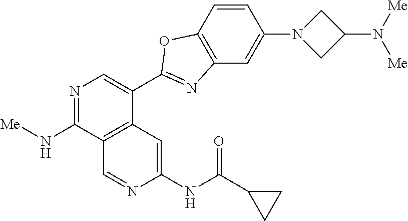 [SMILES: CNc1ncc(-c2nc3cc(N4CC(N(C)C)C4)ccc3o2)c2cc(NC(=O)C3CC3)ncc12] | LCMS (ESI) m/z 458.2, [M + H]⁺ . ¹H NMR (400 MHz, DMSO-d₆) δ 11.03 (s, 1H), 9.67 (s, 1H), 9.43 (s, 1H), 8.88 (s, 1H), 8.65-8.57 (m, 1H), 7.53 (d, J = 8.8 Hz, 1H), 6.71 (d, J = 2.4 Hz, 1H), 6.48 (dd, J = 8.8, 2.4 Hz, 1H), 4.00-3.99 (m, 2H), 3.62-3.59 (m, 2H), 3.28-3.20 (m, 1H), 3.09 (d, J = 4.4 Hz, 3H), 2.22-2.08 (m, 7H), 0.91-0.86 (m, 4H). |
| | N-(5-(5-(3- | |
| | (dimethylamino)azetidin-1- | |
| | yl)benzo[d]oxazol-2-y1)-8- | |
| | (methylamino)-2,7-naphthyridin-3- | |
| | yl)cyclopropanecarboxamide | |
| Example 288 | 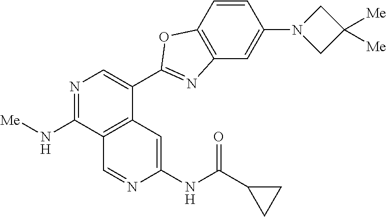 [SMILES: CNc1ncc(-c2nc3cc(N4CC(C)(C)C4)ccc3o2)c2cc(NC(=O)C3CC3)ncc12] | LCMS (ESI) m/z 443.2, [M + H]⁺. ¹H NMR (400 MHz, DMSO-d₆) δ 11.02 (s, 1H), 9.64 (s, 1H), 9.43 (s, 1H), 8.87 (s, 1H), 8.68-8.54 (m, 1H), 7.51 (d, J = 8.8 Hz, 1H), 6.67 (d, J = 2.0 Hz, 1H), 6.50-6.35 (m, 1H), 3.58 (s, 4H), 3.08 (d, J = 4.4 Hz, 3H), 2.15- 2.07 (m, 1H), 1.31 (s, 6H), 0.93-0.82 (m, 4H). |
| | N-(5-(5-(3,3-dimethylazetidin-1- | |
| | yl)benzo[d]oxazol-2-y1)-8- | |
| | (methylamino)-2,7-naphthyridin-3- | |
| | yl)cyclopropanecarboxamide | |

### Example 289: Synthesis of (S)—N-(8-(methylamino)-5-(5-(2-methylmorpholino)benzo[d]oxazol-2-yl)-2-yl)-2,7-naphthyridin-3-yl)cyclopropanecarboxamide

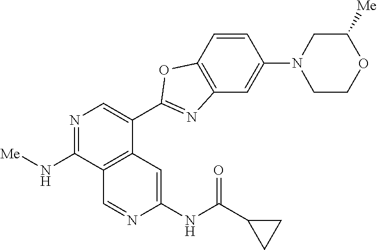 [SMILES: CNc1ncc(-c2nc3cc(N4CCO[C@@H](C)C4)ccc3o2)c2cc(NC(=O)C3CC3)ncc12]

To a stirred mixture of N-(5-(5-bromobenzo[d]oxazol-2-yl)-8-(methy amino)-2, 7-naphthyridin-3 yl)cyclopropanecarboxamide (560.0 mg; 1.278 mmol; 1.00 eq.), EPhos (136.7 mg; 0.256 mmol; 0.20 eq.), EPhos Pd G₄ (234.7 mg; 0.256 mmol; 0.20 eq.). Cs₂CO₃ (1665.2 mg; 5.112 mmol, 4.00 eq.) in 1,4-dioxane (34 mL) was added (S)-2-methylmorpholine (387.7 mg; 3.834 mmol; 3.00 eq.) under nitrogen atmosphere. The resulting mixture was stirred at 100° C. for 24 hours. The desired product was detected via LCMS. The resulting mixture was concentrated under reduced pressure. The residue was purified by flash chromatography on silica gel column using 5-25% of MeOH in CH₂Cl₂ as eluent to provide (5)-4-(8-(methylamino)-5-(5-(2-methylmorpholino)benzo[d]oxazol-2-yl)-2,7-naphthyridin-3-yl)cyclopropanecarboxamide as a yellow solid (143.1 mg, 24.2%). LCMS (ESI) m/z 459.2, [M+H]⁺. ¹H NMR (400 MHz, DMSO-d₆) δ 11.03 (5, 1H), 9.65 (s, 9.43 (s, 1H), 8.88 (s, 1H), 8.68-8.60 (m, 1H), 7.57 (d, J=8.8 Hz, 1H), 7.22 (d, J=2.4 Hz, 1H), 7.10-7.02 (m, 1H), 3.99-3.89 (m, 1H), 3.74-3.59 (m, 3H), 3.55-3.51 (m, 1H), 3.09 (d, J=4.4 Hz, 3H), 2.75-2.66 (m, 1H), 2.41-2.32 m, 1H), 2.12-2.06 (m, 1H), 1.19 (d, J=6.0 Hz, 3H), 0.96-0.82 (m, 4H).

Each compound in Table 13 below was prepared using a similar experimental procedure to prepare Example 289, using N-(5-(5-bromobenzo[d]oxazol-2-yl)-8-(methylamino)-2,7-naphthyridin-3-yl)cyclopropanecarboxamide as the common intermediate and appropriate amine.

TABLE 13

| Example | Structure and Name | MS and ¹H NMR |
|---|---|---|
| Example 290 | 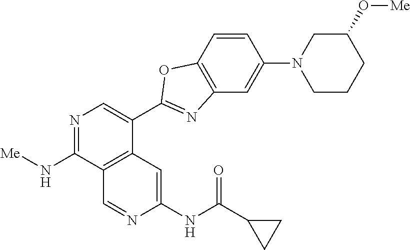 [SMILES: CNc1ncc(-c2nc3cc(N4CCC[C@@H](OC)C4)ccc3o2)c2cc(NC(=O)C3CC3)ncc12] | LCMS (ESI) m/z 473.2, [M + H]⁺. ¹H NMR (400 MHz, DMSO-d₆) δ 11.02 (s, 1H), 9.63 (s, 1H), 9.43 (s, 1H), 8.87 (s, 1H), 8.70-8.45 (m, 1H), 7.55 (d, J = 8.8 Hz, 1H), 7.21 (d, J = 2.4 Hz, 1H), 7.06 (dd, J = 8.8, 2.4 Hz, 1H), 3.69- 3.60 (m, 1H), 3.44-3.37 (m, 2H), 3.34 (s, 3H), 3.09 (d, J = 4.4 Hz, 3H), 2.86- 2.77 (m, 1H), 2.75-2.68 (m, 1H), 2.16- 2.06 (m, 1H), 2.05-1.94 (m, 1H), 1.90-1.77 (m, 1H), 1.67-1.52 (m, 1H), 1.39-1.29 (m, 1H), 0.96-0.81 (m, 4H). |
| | (R)-N-(5-(5-(3-methoxypiperidin-1- | |
| | yl)benzo[d]oxazol-2-yl)-8- | |
| | (methylamino)-2,7-naphthyridin-3- | |
| | yl)cyclopropanecarboxamide | |
| Example 291 | 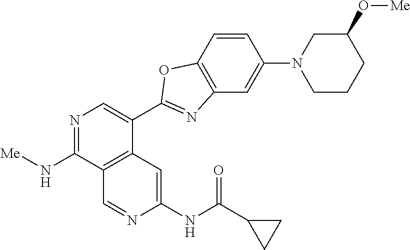 [SMILES: CNc1ncc(-c2nc3cc(N4CCC[C@H](OC)C4)ccc3o2)c2cc(NC(=O)C3CC3)ncc12] | LCMS (ESI) m/z 473.2, [M + H]⁺. ¹H NMR (400 MHz, DMSO-d₆) δ 11.03 (s, 1H), 9.64 (s, 1H), 9.44 (s, 1H), 8.87 (s, 1H), 8.66-8.58 (m, 1H), 7.55 (d, J = 8.8 Hz, 1H), 7.22 (d, J = 2.4 Hz, 1H), 7.06 (dd, J = 8.8, 2.4 Hz, 1H), 3.69- 3.60 (m, 1H), 3.45-3.36 (m, 2H), 3.34 (s, 3H), 3.08 (d, J = 4.4 Hz, 3H), 2.86- 2.75 (m, 1H), 2.74-2.65 (m, 1H), 2.15- 2.05 (m, 1H), 2.05-1.96 (m, 1H), 1.87-1.78 (m, 1H), 1.68-1.55 (m, 1H), 1.40-1.26 (m, 1H), 0.97-0.81 (m, 4H). |
| | (S)-N-(5-(5-(3-methoxypiperidin-1- | |
| | yl)benzo[d]oxazol-2-y1)-8- | |
| | (methylamino)-2,7-naphthyridin-3- | |
| | yl)cyclopropanecarboxamide | |
| Example 292 | 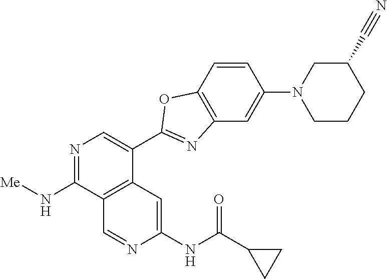 [SMILES: CNc1ncc(-c2nc3cc(N4CCC[C@@H](C#N)C4)ccc3o2)c2cc(NC(=O)C3CC3)ncc12] | LCMS (ESI) m/z 468.2, [M + H]⁺. ¹H NMR (300 MHz, DMSO-d₆) δ 11.01 (s, 1H), 9.64 (s, 1H), 9.43 (s, 1H), 8.88 (s, 1H), 8.67-8.50 (m, 1H), 7.59 (d, J = 8.8 Hz, 1H), 7.27 (d, J = 2.4 Hz, 1H), 7.15-7.98 (m, 1H), 3.41-3.37 (m, 2H), 3.29-3.12 (m, 3H), 3.09 (d, J = 4.4 Hz, 3H), 2.20-2.01 (m, 1H), 1.90- 1.69 (m, 4H), 0.99-0.80 (m, 4H). |
| | (R)-N-(5-(5-(3-cyanopiperidin-1- | |
| | yl)benzo[d]oxazol-2-y1)-8- | |
| | (methylamino)-2,7-naphthyridin-3- | |
| | yl)cyclopropanecarboxamide | |
| Example 293 | 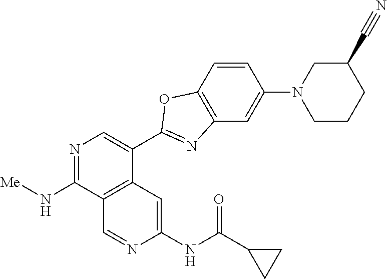 [SMILES: CNc1ncc(-c2nc3cc(N4CCC[C@H](C#N)C4)ccc3o2)c2cc(NC(=O)C3CC3)ncc12] | LCMS (ESI) m/z 468.2, [M + H]⁺. ¹H NMR (400 MHz, DMSO-d₆) δ 11.04 (s, 1H), 9.65 (s, 1H), 9.44 (s, 1H), 8.89 (s, 1H), 8.66-8.60 (m, 1H), 7.59 (d, J = 8.8 Hz, 1H), 7.27 (d, J = 2.4 Hz, 1H), 7.08 (d, J = 8.8, 2.4 Hz, 1H), 3.44- 3.37 (m, 2H), 3.28-3.11 (m, 3H), 3.09 (d, J = 4.4 Hz, 3H), 2.14-2.06 (m, 1H), 1.91-1.79 (m, 3H), 1.73-1.69 (m, 1H), 0.95-0.82 (m, 4H). |
| | (S)-N-(5-(5-(3-cyanopiperidin-1- | |
| | yl)benzo[djoxazol-2-yl)-8- | |
| | (methylamino)-2,7-naphthyridin-3- | |
| | yl)cyclopropanecarboxamide | |
| Example 294 | 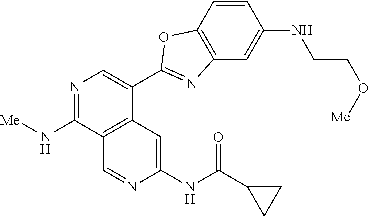 [SMILES: CNc1ncc(-c2nc3cc(NCCOC)ccc3o2)c2cc(NC(=O)C3CC3)ncc12] | LCMS (ESI) m/z 433.2, [M + H]⁺. ¹H NMR (400 MHz, Methanol-d₄) δ 9.71 (s, 1H), 9.25 (s, 1H), 8.74 (s, 1H), 7.46 (d, J = 8.8 Hz, 1H), 7.13 (d, J = 2.4 Hz, 1H), 6.86 (d, J = 8.8, 2.4 Hz, 1H), 3.71- 3.64 (m, 2H), 3.44 (s, 3H), 3.41-3.35 (m, 2H), 3.17 (s, 3H), 2.04-1.94 (m, 1H), 1.15-1.06 (m, 2H), 1.02-0.93 (m, 2H). |
| | N-(5-(5-((2- | |
| | methoxyethyl)amino)benzo[d]oxazol- | |
| | 2-yl)-8-(methylamino)-2,7- | |
| | naphthyridin-3- | |
| | yl)cyclopropanecarboxamide | |
| Example 295 | 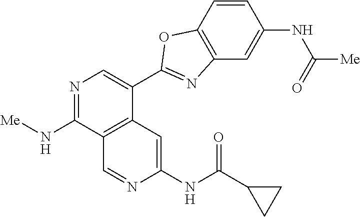 [SMILES: CNc1ncc(-c2nc3cc(NC(C)=O)ccc3o2)c2cc(NC(=O)C3CC3)ncc12] | LCMS (ESI) m/z 417.2, [M + H]⁺. ¹H NMR (400 MHz, DMSO-d₆) δ 11.05 (s, 1H), 10.09 (s, 1H), 9.76 (s, 1H), 9.44 (s, 1H), 8.91 (s, 1H), 8.70-8.59 (m, 1H), 8.16 (d, J = 2.0 Hz, 1H), 7.64 (d, J = 8.8 Hz, 1H), 7.9-7.39 (m, 1H), 3.09 (d, J = 4.4 Hz, 3H), 2.16-2.03 (m, 4H), 0.99-0.90 (m, 2H), 0.89-0.75 (m, 2H). |
| | N-(5-(5-acetamidobenzo[d]oxazol- | |
| | 2-yl)-8-(methylamino)-2,7- | |
| | naphthyridin-3- | |
| | yl)cyclopropanecarboxamide | |
| Example 296 | 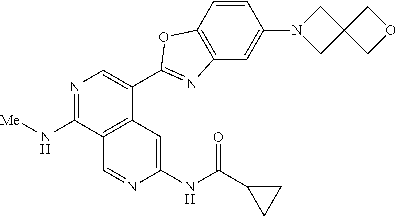 [SMILES: CNc1ncc(-c2nc3cc(N4CC5(COC5)C4)ccc3o2)c2cc(NC(=O)C3CC3)ncc12] | LCMS (ESI) m/z 457.2, [M + H]⁺. ¹H NMR (400 MHz, DMSO-d₆) δ 11.03 (s, 1H), 9.65 (s, 1H), 9.43 (s, 1H), 8.87 (s, 1H), 8.68 - 8.58 (m, 1H), 7.53 (d, J = 8.8 Hz, 1H), 6.72 (d, J = 2.0 Hz, 1H), 6.51-6.43 (m, 1H), 4.75 (s, 4H), 4.03 (s, 4H), 3.08 (d, J = 4.4 Hz, 3H), 2.16- 2.05 (m, 1H), 0.97-0.90 (m, 2H), 0.89- 0.80 (m, 2H). |
| | N-(5-(5-(2-oxa-6- | |
| | azaspiro[3.3]heptan-6- | |
| | yl)benzo[d]oxazol-2-y1)-8- | |
| | (methylamino)-2,7-naphthyridin-3- | |
| | yl)cyclopropanecarboxamide | |
| Example 297 | 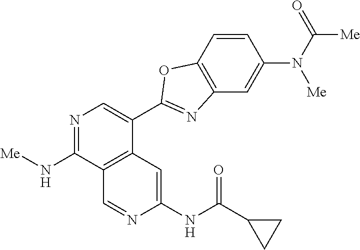 [SMILES: CNc1ncc(-c2nc3cc(N(C)C(C)=O)ccc3o2)c2cc(NC(=O)C3CC3)ncc12] | LCMS (ESI) m/z 431.2, [M + H]⁺. ¹H NMR (400 MHz, DMSO-d₆) δ 11.06 (s, 1H), 9.66 (s, 1H), 9.45 (s, 1H), 8.93 (s, 1H), 8.74-8.67 (m, 1H), 7.82-7.72 (m, 2H), 7.35 (d, J = 8.4 Hz, 1H), 3.22 (s, 3H), 3.10 (d, J = 4.4 Hz, 3H), 2.14- 2.05 (m, 1H), 1.80 (s, 3H), 0.95-0.82 (m, 4H). |
| | N-(5-(5-(N- | |
| | methylacetamido)benzo[d]oxazol- | |
| | 2-yl)-8-(methylamino)-2,7- | |
| | naphthyridin-3- | |
| | y1)cyclopropanecarboxamide | |
| Example 298 | 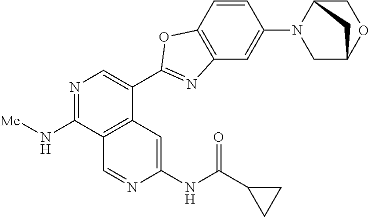 [SMILES: CNc1ncc(-c2nc3cc(N4C[C@@H]5C[C@H]4CO5)ccc3o2)c2cc(NC(=O)C3CC3)ncc12] | LCMS (ESI) m/z 457.2, [M + H]⁺. ¹H NMR (400 MHz, DMSO-d₆) δ 11.03 (s, 1H), 9.62 (s, 1H), 9.44 (s, 1H), 8.87 (s, 1H), 8.68-8.57 (m, 1H), 7.52 (d, J = 8.8 Hz, 1H), 6.92 (d, J = 2.0 Hz, 1H), 6.68 (dd, J = 8.8, 2.0 Hz, 1H), 4.70- 4.61 (m, 2H), 3.83-3.72 (m, 2H), 3.60 (d, J = 8.8 Hz, 1H), 3.08 (d, J = 4.4 Hz, 3H), 3.02 (d, J = 8.8 Hz, 1H), 2.15- 2.05 (m, 1H), 2.02-1.94 (m, 1H), 1.90- 1.83 (m, 1H), 0.95-0.81 (m, 4H). |
| | N-(5-(5-((15,4S)-2-oxa-5- | |
| | azabicyclo[2.2.1]heptan-5- | |
| | yl)benzo[d]oxazol-2-y1)-8- | |
| | (methylamino)-2,7-naphthyridin-3- | |
| | yl)cyclopropanecarboxamide | |
| Example 299 | 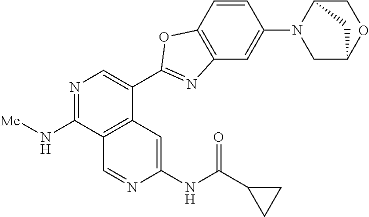 [SMILES: CNc1ncc(-c2nc3cc(N4C[C@H]5C[C@@H]4CO5)ccc3o2)c2cc(NC(=O)C3CC3)ncc12] | LCMS (ESI) m/z 457.2, [M + H]⁺. ¹H NMR (400 MHz, DMSO-d₆) δ 11.02 (s, 1H), 9.62 (s, 1H), 9.44 (s, 1H), 8.87 (s, 1H), 8.77-8.39 (m, 1H), 7.53 (d, J = 8.8 Hz, 1H), 6.92 (d, J = 2.0 Hz, 1H), 6.73-6.60 (m, 1H), 4.64 (d, J = 8.4 Hz, 2H), 3.83-3.71 (m, 2H), 3.60 (d, J = 9.2 Hz, 1H), 3.09 (d, J = 4.0 Hz, 3H), 3.02 (d, J = 9.2 Hz, 1H), 2.18-2.02 (m, 1H), 2.01-1.94 (m, 1H), 1.89-1.83 (m, 1H), 0.95 - 0.84 (m, 4H). |
| | N-(5-(5-((1R,4R)-2-oxa-5- | |
| | azabicyclo[2.2.1]heptan-5- | |
| | yl)benzo[d]oxazol-2-y1)-8- | |
| | (methylamino)-2,7-naphthyridin-3- | |
| | yl)cyclopropanecarboxamide | |
| Example 300 | 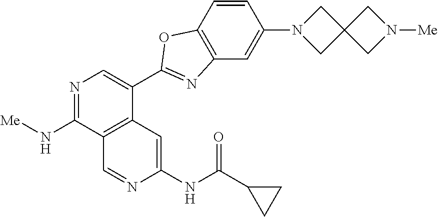 [SMILES: CNc1ncc(-c2nc3cc(N4CC5(CN(C)C5)C4)ccc3o2)c2cc(NC(=O)C3CC3)ncc12] | LCMS (ESI) m/z 470.2, [M + H]⁺. ¹H NMR (400 MHz, DMSO-d₆) δ 11.04 (s, 1H), 9.64 (s, 1H), 9.43 (s, 1H), 8.87 (s, 1H), 8.69-8.54 (m, 1H), 7.52 (d, J = 8.8 Hz, 1H), 6.70 (d, J = 2.4 Hz, 1H), 6.50-6.41 (m, 1H), 3.92 (s, 4H), 3.39 (s, 4H), 3.08 (d, J = 4.4 Hz, 3H), 2.27 (s, 3H), 2.12-2.02 (m, 1H), 0.97-0.81 (m, 4H). |
| | N-(5-(5-(6-methyl-2,6- | |
| | diazaspiro[3.3]heptan-2- | |
| | yl)benzo[d]oxazol-2-yl)-8- | |
| | (methylamino)-2,7-naphthyridin-3- | |
| | yl)cyclopropanecarboxamide | |
| Example 301 | 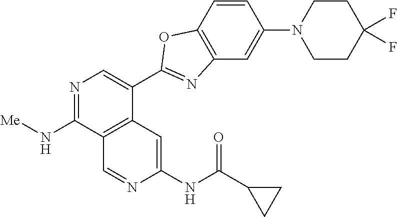 [SMILES: CNc1ncc(-c2nc3cc(N4CCC(F)(F)CC4)ccc3o2)c2cc(NC(=O)C3CC3)ncc12] | LCMS (ESI) m/z 479.2, [M + H]⁺. ¹H NMR (400 MHz, DMSO-d₆) δ 11.04 (s, 1H), 9.64 (s, 1H), 9.44 (s, 1H), 8.88 (s, 1H), 8.68-8.61 (m, 1H), 7.59 (d, J = 8.8 Hz, 1H), 7.31 (d, J = 2.4 Hz, 1H), 7.11 (d, J = 8.8 Hz, 1H), 3.39-3.33 (m, 4H), 3.09 (d, J = 4.4 Hz, 3H), 2.20- 2.06 (m, 5H), 0.95-0.81 (m, 4H). |
| | N-(5-(5-(4,4-difluoropiperidin-1- | |
| | yl)benzo[d]oxazol-2-y1)-8- | |
| | (methylamino)-2,7-naphthyridin-3- | |
| | yl)cyclopropanecarboxamide | |
| Example 302 | 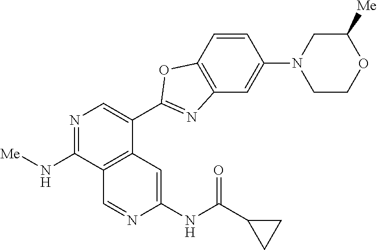 [SMILES: CNc1ncc(-c2nc3cc(N4CCO[C@H](C)C4)ccc3o2)c2cc(NC(=O)C3CC3)ncc12] | LCMS (ESI) m/z 459.2, [M + H]⁺. ¹H NMR (400 MHz, DMSO-d₆) δ 11.02 (s, 1H), 9.64 (s, 1H), 9.43 (s, 1H), 8.88 (s, 1H), 8.67-8.58 (m, 1H), 7.57 (d, J = 8.8 Hz, 1H), 7.22 (d, J = 2.4 Hz, 1H), 7.06 (d, J = 8.8, 2.4 Hz, 1H), 3.98- 3.90 (m, 1H), 3.77-3.57 (m, 3H), 3.58- 3.49 (m, 1H), 3.09 (d, J = 4.4 Hz, 3H), 2.78-2.67 (m, 1H), 2.44-2.34 (m, 1H), 2.15-2.06 (m, 1H), 1.19 (d, J = 6.4 Hz, 3H), 0.98-0.81 (m, 4H). |
| | (R)-N-(8-(methylamino)-5-(5-(2- | |
| | methylmorpholino)benzo[d]oxazol- | |
| | 2-yl)-2,7-naphthyridin-3- | |
| | yl)cyclopropanecarboxamide | |
| Example 303 | 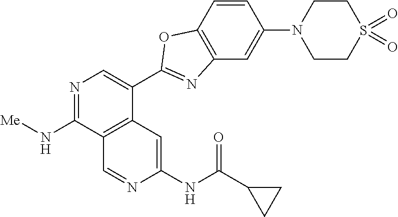 [SMILES: CNc1ncc(-c2nc3cc(N4CCS(=O)(=O)CC4)ccc3o2)c2cc(NC(=O)C3CC3)ncc12] | LCMS (ESI) m/z 493.2, [M + H]⁺. ¹H NMR (400 MHz, DMSO-d₆) δ 11.03 (s, 1H), 9.62 (s, 1H), 9.44 (s, 1H), 8.89 (s, 1H), 8.73-8.49 (m, 1H), 7.61 (d, J = 8.8 Hz, 1H), 7.38 (d, J = 2.4 Hz, 1H), 7.25-7.00 (m, 1H), 3.82-3.72 (m, 4H), 3.25-3.17 (m, 4H), 3.09 (d, J = 4.4 Hz, 3H), 2.18-2.04 (m, 1H), 0.97- 0.80 (m, 4H). |
| | N-(5-(5-(1,1- | |
| | dioxidothiomorpholino)benzo[d]ox | |
| | azol-2-yl)-8-(methylamino)-2,7- | |
| | naphthyridin-3- | |
| | yl)cyclopropanecarboxamide | |
| Example 304 | 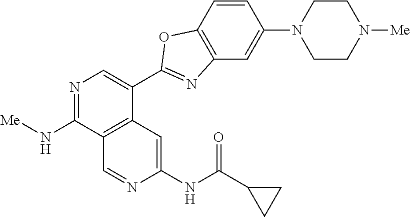 [SMILES: CNc1ncc(-c2nc3cc(N4CCN(C)CC4)ccc3o2)c2cc(NC(=O)C3CC3)ncc12] | LCMS (ESI) m/z 458.2, [M + H]⁺. ¹H NMR (400 MHz, DMSO-d₆) δ 11.03 (s, 1H), 9.64 (s, 1H), 9.44 (s, 1H), 8.88 (s, 1H), 8.66-8.58 (m, 1H), 7.56 (d, J = 8.8 Hz, 1H), 7.20 (d, J = 2.0 Hz, 1H), 7.09-7.01 (m, 1H), 3.33-3.30 (m, 4H), 3.26-3.14 (m, 4H), 3.09 (d, J = 4.4 Hz, 3H), 2.25 (s, 3H), 2.19-2.07 (m, 1H), 0.97-0.80 (m, 4H). |
| | N-(8-(methylamino)-5-(5-(4- | |
| | methylpiperazin-1- | |
| | yI)benzo[d]oxazol-2-y1)-2,7- | |
| | naphthyridin-3- | |
| | yl)cyclopropanecarboxamide | |
| Example 305 | 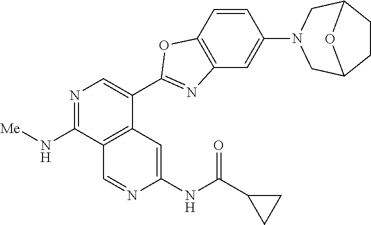 [SMILES: CNc1ncc(-c2nc3cc(N4CC5CCC(C4)O5)ccc3o2)c2cc(NC(=O)C3CC3)ncc12] | LCMS (ESI) m/z 471.2, [M + H]⁺. ¹H NMR (400 MHz, DMSO-d₆) δ 11.03 (s, 1H), 9.66 (s, 1H), 9.43 (s, 1H), 8.88 (s, 1H), 8.68-8.59 (m, 1H), 7.55 (d, J = 8.8 Hz, 1H), 7.12 (d, J = 2.4 Hz, 1H), 7.00-6.91 (m, 1H), 4.50-4.45 (m, 2H), 3.46 (d, J = 11.2 Hz, 2H), 3.09 (d, J = 4.4 Hz, 3H), 2.87 (d, J = 11.2 Hz, 2H), 2.17-2.04 (m, 1H), 1.97-1.80 (m, 4H), 0.97-0.81 (m, 4H). |
| | N-(5-(5-(8-oxa-3- | |
| | azabicyclo[3.2.1]octan-3- | |
| | ylbenzo[d]oxazol-2-yl)-8- | |
| | (methylamino)-2,7-naphthyridin-3- | |
| | yl)cyclopropanecarboxamide | |
| Example 306 | 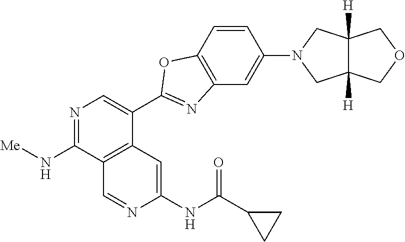 [SMILES: CNc1ncc(-c2nc3cc(N4C[C@H]5COC[C@H]5C4)ccc3o2)c2cc(NC(=O)C3CC3)ncc12] | LCMS (ESI) m/z 471.2, [M + H]⁺. ¹H NMR (400 MHz, DMSO-d₆) δ 11.03 (s, 1H), 9.67 (s, 1H), 9.43 (s, 1H), 8.88 (s, 1H), 8.64-8.57 (m, 1H), 7.53 (d, J = 8.8 Hz, 1H), 6.90 (d, J = 2.4 Hz, 1H), 6.73 (dd, J = 8.8, 2.4 Hz, 1H), 3.92- 3.84 (m, 2H), 3.63-3.55 (m, 2H), 3.44- 3.36 (m, 2H), 3.29-3.21 (m, 2H), 3.08 (d, J = 4.4 Hz, 3H), 3.06-2.99 (m, 2H), 2.16-2.05 (m, 1H), 0.96-0.81 (m, 4H). |
| | N-(8-(methylamino)-5-(5- | |
| | ((3aR,6a.S)-tetrahydro-1H-furo[3,4- | |
| | c]pyrrol-5(3H)-yl)benzo[d]oxazol- | |
| | 2-y1)-2,7-naphthyridin-3- | |
| | yl)cyclopropanecarboxamide | |
| Example 307 | 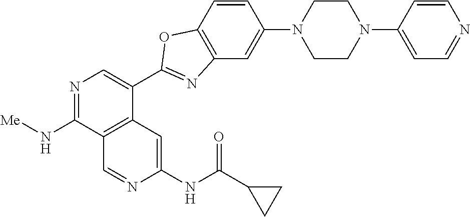 [SMILES: CNc1ncc(-c2nc3cc(N4CCN(c5ccncc5)CC4)ccc3o2)c2cc(NC(=O)C3CC3)ncc12] | LCMS (ESI) m/z 521.2, [M + H]⁺. ¹H NMR (400 MHz, DMSO-d₆) δ 11.04 (s, 1H), 9.66 (s, 1H), 9.44 (s, 1H), 8.89 (s, 1H), 8.67-8.52 (m, 1H), 8.20 (d, J = 5.6 Hz, 2H), 7.61 (d, J = 8.8 Hz, 1H), 7.30 (d, J = 2.4 Hz, 1H), 7.21-7.06 (m, 1H), 6.91 (d, J = 5.6 Hz, 2H), 3.59- 3.48 (m, 4H), 3.29 (s, 4H), 3.09 (d, J = 4.4 Hz, 3H), 2.17-2.04 (m, 1H), 0.95- 0.90 (m, 2H), 0.90-0.81 (m, 2H). |
| | N-(8-(methylamino)-5-(5-(4- | |
| | (pyridin-4-yl)piperazin-1- | |
| | yl)benzo[d]oxazol-2-yl)-2,7- | |
| | naphthyridin-3- | |
| | yl)cyclopropanecarboxamide | |
| Example 308 | 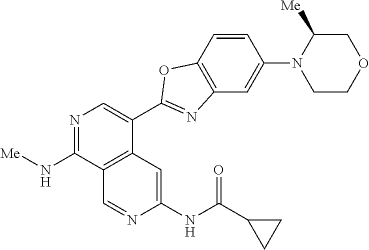 [SMILES: CNc1ncc(-c2nc3cc(N4CCOC[C@@H]4C)ccc3o2)c2cc(NC(=O)C3CC3)ncc12] | LCMS (ESI) m/z 459.2, [M + H]⁺ ¹H NMR (400 MHz, DMSO-d₆) δ 11.04 (s, 1H), 9.65 (s, 1H), 9.44 (s, 1H), 8.88 (s, 1H), 8.67-8.60 (m, 1H), 7.59 (d, J = 8.8 Hz, 1H), 7.22 (d, J = 2.0 Hz, 1H), 7.04 (dd, J = 8.8, 2.0 Hz, 1H), 3.95- 3.87 (m, 1H), 3.87-3.75 (m, 2H), 3.70- 3.59 (m, 2H), 3.17-3.03 (m, 5H), 2.16-2.05 (m, 1H), 0.96 (d, J = 6.4 Hz, 3H), 0.94-0.83 (m, 4H). |
| | (S)-N-(8-(methylamino)-5-(5-(3- | |
| | methylmorpholino)benzo[d]oxazol- | |
| | 2-yl)-2,7-naphthyridin-3- | |
| | yl)cyclopropanecarboxamide | |
| Example 309 | 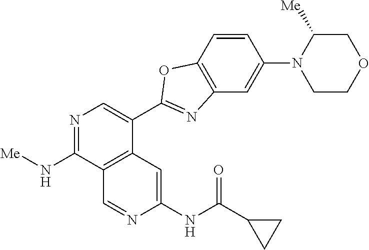 [SMILES: CNc1ncc(-c2nc3cc(N4CCOC[C@H]4C)ccc3o2)c2cc(NC(=O)C3CC3)ncc12] | LCMS (ESI) m/z 459.2, [M + H]⁺. ¹H NMR (400 MHz, DMSO-d₆) δ 11.03 (s, 1H), 9.64 (s, 1H), 9.44 (s, 1H), 8.88 (s, 1H), 8.68-8.50 (m, 1H), 7.59 (d, J = 8.8 Hz, 1H), 7.22 (d, J = 2.0 Hz, 1H), 7.11-6.99 (m, 1H), 3.93-3.90 (m, 1H), 3.84-3.79 (m, 2H), 3.69-3.61 (m, 2H), 3.12-3.08 (m, 5H), 2.15- 2.09 (m, 1H), 0.96 (d, J = 6.4 Hz, 3H), 0.94-0.89 (m, 2H), 0.89-0.84 (m, 2H). |
| | (R)-N-(8-(methylamino)-5-(5-(3- | |
| | methylmorpholino)benzo[d]oxazol- | |
| | 2-yl)-2,7-naphthyridin-3- | |
| | yl)cyclopropanecarboxamide | |
| Example 310 | 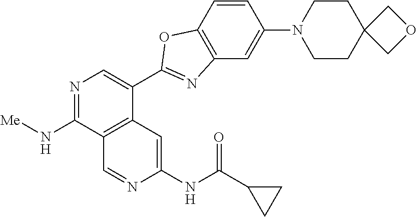 [SMILES: CNc1ncc(-c2nc3cc(N4CCC5(CC4)COC5)ccc3o2)c2cc(NC(=O)C3CC3)ncc12] | LCMS (ESI) m/z 485.2, [M + H]⁺. ¹H NMR (400 MHz, DMSO-d₆) δ 11.02 (s, 1H), 9.64 (s, 1H), 9.43 (s, 1H), 8.87 (s, 1H), 8.70-8.45 (m, 1H), 7.55 (d, J = 8.8 Hz, 1H), 7.21 (d, J = 2.4 Hz, 1H), 7.11-6.90 (m, 1H), 4.37 (s, 4H), 3.19- 3.00 (m, 7H), 2.20-2.02 (m, 1H), 2.00-1.80 (m, 4H), 0.94-0.82 (m, 4H). |
| | N-(5-(5-(2-oxa-7- | |
| | azaspiro[3.5]nonan-7- | |
| | yl)benzo[d]oxazol-2-y1)~8- | |
| | (methylamino)-2,7-naphthyridin-3- | |
| | yl)cyclopropanecarboxamide | |
| Example 311 | 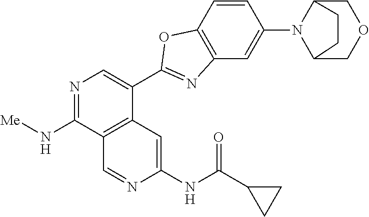 [SMILES: CNc1ncc(-c2nc3cc(N4C5CCC4COC5)ccc3o2)c2cc(NC(=O)C3CC3)ncc12] | LCMS (ESI) m/z 471.2, [M + H]⁺. ¹H NMR (400 MHz, DMSO-d₆) δ 11.02 (s, 1H), 9.60 (s, 1H), 9.44 (s, 1H), 8.87 (s, 1H), 8.64-8.59 (m, 1H), 7.54 (d, J = 8.8 Hz, 1H), 7.16 (d, J = 2.4 Hz, 1H), 6.98-6.91 (m, 1H), 4.30-4.23 (m, 2H), 3.79 (d, J = 10.8 Hz, 2H), 3.48 (d, J = 10.8 Hz, 2H), 3.09 (d, J = 4.4 Hz, 3H), 2.17-2.06 (m, 1H), 1.98-1.89 (m, 4H), 0.95-0.83 (m, 4H). |
| | N-(5-(5-(3-oxa-8- | |
| | azabicyclo[3.2.1]octan-8- | |
| | yl)benzo[d]oxazol-2-y1)-8- | |
| | (methylamino)-2,7-naphthyridin-3- | |
| | yl)cyclopropanecarboxamide | |
| Example 312 | 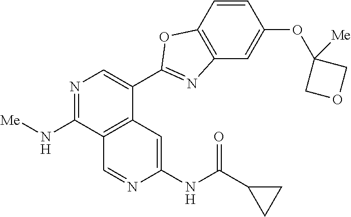 [SMILES: CNc1ncc(-c2nc3cc(OC4(C)COC4)ccc3o2)c2cc(NC(=O)C3CC3)ncc12] | LCMS (ESI) m/z 446.2, [M + H]⁺. ¹H NMR (400 MHz, DMSO-d₆) δ 11.05 (s, 1H), 9.73 (s, 1H), 9.45 (s, 1H), 8.93 (s, 1H), 8.70-8.64 (m, 1H), 7.77-7.68 (m, 2H), 7.39 (dd, J = 8.4, 1.6 Hz, 1H), 4.66 (s, 2H), 4.21 (s, 2H), 3.10 (d, J = 4.4 Hz, 3H), 2.16-2.10 (m, 1H), 2.09 (s, 3H), 0.96-0.80 (m, 4H). |
| | N-(8-(methylamino)-5-(5-((3- | |
| | methyloxetan-3- | |
| | yl)oxy)benzo[d]oxazol-2-yl)-2,7- | |
| | naphthyridin-3- | |
| | yl)cyclopropanecarboxamide | |
| Example 313 | 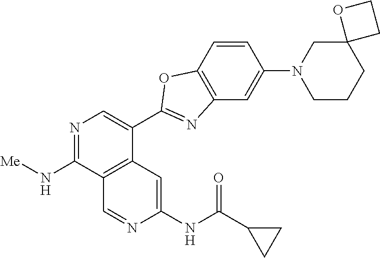 [SMILES: CNc1ncc(-c2nc3cc(N4CCCC5(CCO5)C4)ccc3o2)c2cc(NC(=O)C3CC3)ncc12] | LCMS (ESI) m/z 485.2, [M + H]⁺. ¹H NMR (300 MHz, DMSO-d₆) δ 11.01 (s, 1H), 9.64 (s, 1H), 9.44 (s, 1H), 8.88 (s, 1H), 8.66-8.45 (m, 1H), 7.57 (d, J = 8.4 Hz, 1H), 7.25 (d, J = 2.4 Hz, 1H), 7.10 (dd, J = 8.4, 2.4 Hz, 1H), 4.60- 4.43 (m, 2H), 3.55-3.48 (m, 1H), 3.21- 3.02 (m, 5H), 3.00-2.93 (m, 1H), 2.45-2.39 (m, 1H), 2.18-2.10 (m, 1H), 1.96-1.91 (m, 1H), 1.82-1.55 (m, 3H), 1.26-1.22 (m, 1H), 0.98- 0.80 (m, 4H). |
| | N-(5-(5-(1-oxa-6- | |
| | azaspiro[3.5]nonan-6- | |
| | yl)benzo[d]oxazol-2-yl)-8- | |
| | (methylamino)-2,7-naphthyridin-3- | |
| | yl)cyclopropanecarboxamide | |
| Example 314 | 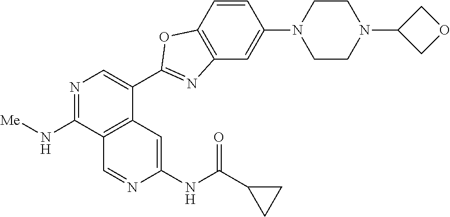 [SMILES: CNc1ncc(-c2nc3cc(N4CCN(C5COC5)CC4)ccc3o2)c2cc(NC(=O)C3CC3)ncc12] | LCMS (ESI) m/z 500.2, [M + H]⁺. ¹H NMR (400 MHz, DMSO-d₆) δ 11.02 (s, 1H), 9.64 (s, 1H), 9.44 (s, 1H), 8.88 (s, 1H), 8.70-8.49 (m, 1H), 7.57 (d, J = 8.8 Hz, 1H), 7.22 (d, J = 2.4 Hz, 1H), 7.15-6.95 (m, 1H), 4.63-4.55 (m, 2H), 4.54-4.38 (m, 2H), 3.52-3.43 (m, 1H), 3.27-3.18 (m, 4H), 3.09 (d, J = 4.4 Hz, 3H), 2.50-2.43 (m, 4H), 2.16- 2.07 (m, 1H), 0.93-0.83 (m, 4H). |
| | N-(8-(methylamino)-5-(5-(4- | |
| | (oxetan-3-yl)piperazin-1- | |
| | yl)benzo[d]oxazol-2-yl)-2,7- | |
| | naphthyridin-3- | |
| | yl)cyclopropanecarboxamide | |
| Example 315 | 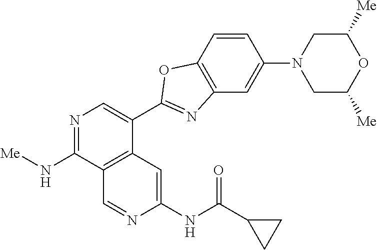 [SMILES: CNc1ncc(-c2nc3cc(N4C[C@@H](C)O[C@@H](C)C4)ccc3o2)c2cc(NC(=O)C3CC3)ncc12] | LCMS (ESI) m/z 473.2, [M + H]⁺. ¹H NMR (400 MHz, DMSO-d₆) δ 11.00 (s, 1H), 9.63 (s, 1H), 9.43 (s, 1H), 8.88 (s, 1H), 8.70-8.40 (m, 1H), 7.57 (d, J = 8.8 Hz, 1H), 7.22 (d, J = 2.4 Hz, 1H), 7.12-6.90 (m, 1H), 3.87-3.70 (m, 2H), 3.61 (d, J = 6.8 Hz, 2H), 3.09 (d, J = 4.4 Hz, 3H), 2.40-2.25 (m, 2H), 2.20-2.04 (m, 1H), 1.19 (d, J = 6.0 Hz, 6H), 0.97-0.82 (m, 4H). |
| | N-(5-(5-((2R,6S)~2,6- | |
| | dimethylmorpholino)benzo[d]oxazol- | |
| | 2-yl)-8-(methylamino)-2,7- | |
| | naphthyridin-3- | |
| | yl)cyclopropanecarboxamide | |
| Example 316 | 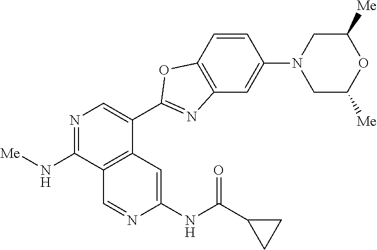 [SMILES: CNc1ncc(-c2nc3cc(N4C[C@@H](C)O[C@H](C)C4)ccc3o2)c2cc(NC(=O)C3CC3)ncc12] | LCMS (ESI) m/z 473.2, [M + H]⁺. ¹H NMR (300 MHz, DMSO-d₆) δ 11.01 (s, 1H), 9.64 (s, 1H), 9.44 (s, 1H), 8.89 (s, 1H), 8.69-8.58 (m, 1H), 7.57 (d, J = 8.7 Hz, 1H), 7.21 (d, J = 2.4 Hz, 1H), 7.04 (dd, J = 8.7, 2.4 Hz, 1H), 4.14- 4.08 (m, 2H), 3.28-3.19 (m, 2H), 3.09 (d, J = 4.4 Hz, 3H), 2.94-2.84 (m, 2H), 2.16-2.06 (m, 1H), 1.31-1.21 (m, 6H), 0.96-0.83 (m, 4H). |
| | trans racemate | |
| | N-(5-(5-(2,6- | |
| | dimethylmorpholino)benzo[d]oxazol- | |
| | 2-y1)-8-(methylamino)-2,7- | |
| | naphthyridin-3- | |
| | yl)cyclopropanecarboxamide (trans | |
| | racemate) | |
| Example 317 | 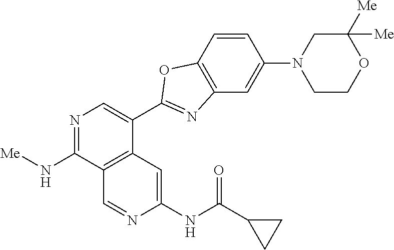 [SMILES: CNc1ncc(-c2nc3cc(N4CCOC(C)(C)C4)ccc3o2)c2cc(NC(=O)C3CC3)ncc12] | LCMS (ESI) m/z 473.2, [M + H]⁺. ¹H NMR (400 MHz, DMSO-d₆) δ 11.02 (s, 1H), 9.65 (s, 1H), 9.43 (s, 1H), 8.89 (s, 1H), 8.68-8.58 (m, 1H), 7.57 (d, J = 8.8 Hz, 1H), 7.21 (d, J = 2.4 Hz, 1H), 7.10-6.97 (m, 1H), 3.88-3.62 (m, 2H), 3.12-3.04 (m, 5H), 2.98 (s, 2H), 2.2-2.04 (m, 1H), 1.28 (s, 6H), 0.97- 0.86 (m, 4H). |
| | N-(5-(5-(2,2- | |
| | dimethylmorpholino)benzo[d]oxazol- | |
| | 2-yl)-8-(methylamino)-2,7- | |
| | naphthyridin-3- | |
| | yl)cyclopropanecarboxamide | |
| Example 318 | 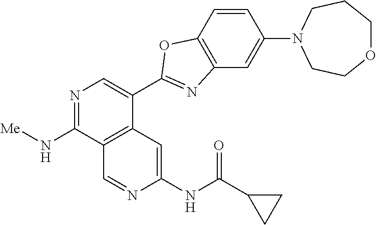 [SMILES: CNc1ncc(-c2nc3cc(N4CCCOCC4)ccc3o2)c2cc(NC(=O)C3CC3)ncc12] | LCMS (ESI) m/z 459.2, [M + H]⁺. ¹H NMR (400 MHz, DMSO-d₆) δ 11.02 (s, 1H), 9.59 (s, 1H), 9.43 (s, 1H), 8.86 (s, 1H), 8.64-8.55 (m, 1H), 7.51 (d, J = 8.8 Hz, 1H), 7.00 (d, J = 2.4 Hz, 1H), 6.87-6.80 (m, 1H), 3.83-3.73 (m, 2H), 3.68-3.56 (m, 6H), 3.08 (d, J = 4.4 Hz, 3H), 2.14-2.05 (m, 1H), 2.02- 1.91 (m, 2H), 0.95-0.89 (m, 2H), 0.88-0.82 (m, 2H). |
| | N-(5-(5-(1,4-oxazepan-4- | |
| | yl)benzo[d]oxazol-2-yl)-8- | |
| | (methylamino)-2,7-naphthyridin-3- | |
| | yl)cyclopropanecarboxamide | |

### Example 319: Synthesis of N-(5-(5-(3-(2,2-difluoroethyl)azetidin-1-yl)benzo[d]oxazol-2-yl)-8-(methylamino)-2,7-naphthyridin-3-yl)cyclopropanecarboxamide

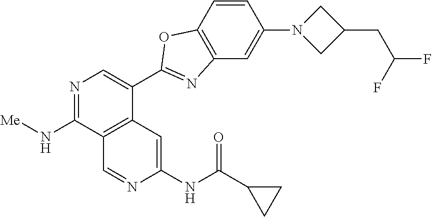 [SMILES: CNc1ncc(-c2nc3cc(N4CC(CC(F)F)C4)ccc3o2)c2cc(NC(=O)C3CC3)ncc12]

### Step 1: tert-butyl 3-(2-oxoethyl)azetidine-1-carboxylate

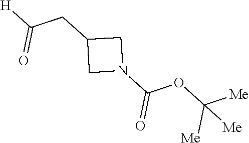 [SMILES: CC(C)(C)OC(=O)N1CC(CC=O)C1]

To a solution of tert-butyl 3-(2-methoxy-2-oxoethyl)azetidine-1-carboxylate (5.0 g; 21.808 mmol, 1.00 eq.) in CH₂Cl₂ (100 mL) was added DIBAL-H (1 M in hexane, 25 mL) drop-wise at −78° C. under nitrogen atmosphere. The resulting mixture was stirred at −78° C. for 10 min. The reaction was quenched by the addition of sodium sulfate decahydrate. The resulting mixture was filtered, the filtrate was concentrated under reduced pressure. The residue was purified by flash chromatography on silica gel column using 10-30% of EtOAc in petroleum ether as eluent to provide tort-butyl 3-(2-oxoethyl)azetidine-1-carboxylate as a light yellow oil (2.3 g, 52.9%). ¹H NMR (400 MHz, DMSO-d₆) δ 9.63 (s, 1H), 4.00-3.86 (m, 2H), 3.58-3.42 (m, 2H), 2.92-2.75 (m, 3H), 1.37 (s, 9H).

### Step 2: tert-butyl 3-(2,2-difluoroethyl)azetidine-1-carboxylate

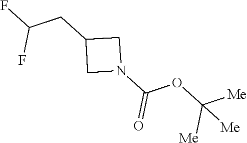 [SMILES: CC(C)(C)OC(=O)N1CC(CC(F)F)C1]

To a solution of tert-butyl 3-(2-oxoethyl)azetidine-1-carboxylate (2.30 g; 11,543 mmol; 1.00 eq.) in CH₂Cl₂ (100 mL) was added BAST (7.30 g; 32.996 mmol; 2.86 eq.) dropwise at 0° C. under nitrogen atmosphere. The resulting mixture was stirred at room temperature for 2 hours. The reaction was quenched by the addition of sat. NaHCO₃ aqueous solution (200 mL) and extracted with CH₂Cl₂ (3×300 mL). The combined organic layers were washed with water (100 mL), dried over anhydrous Na₂SO₄. After filtration, the filtrate was concentrated under reduced pressure. The residue was purified by flash chromatography on silica gel column using 10-30% of EtOAc in petroleum ether as eluent to provide tert-butyl 3-(2,2-difluoroethyl)azetidine-1-carboxylate as a light yellow oil (1.8 g, 70.5%).

### Step 3: 3-(2,2-difluoroethyl)azetidine hydrochloride

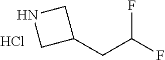

To a solution of tert-butyl 3-(2,2-difluoroethyl)azetidine-1-carboxylate (1.3 g, 5.876 mmol, 1 eq.) in MeOH (1.5 mL) was added 1-ICI (gas) (4 M in dioxane, 5 mL) at room temperature. The resulting mixture was stirred at room temperature for 1 hour under nitrogen atmosphere. The resulting mixture was concentrated under reduced pressure to afford 3-(2,2-difluoroethyl)azetidine hydrochloride as a light yellow oil (800.0 mg, 86.4%). LCMS (ESI) m/z 122.1, [M+H]⁺.

### Step 4: N-(5-(5-(3-(2,2-difluoroethyl)azetidin-1-yl)benzo[d]oxazol-2-yl)-8-(methylamino)-2,7-naphthyridin-3-yl)cyclopropanecarboxamide

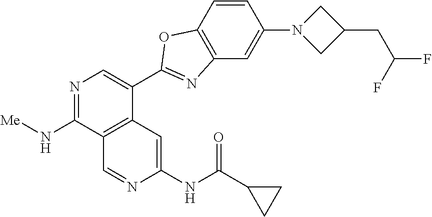 [SMILES: CNc1ncc(-c2nc3cc(N4CC(CC(F)F)C4)ccc3o2)c2cc(NC(=O)C3CC3)ncc12]

N-(5-(5-(3-(2,2-difluoroethyl)azetidin-1-yl)benzo[d]oxazol-2-yl)-8-(methylamino)-2,7-naphthyridin-3-yl)cyclopropanecarboxamide was synthesized using a similar procedure that was previously described in Example 277 by using 14′-(5-(5-bromobenzo[d]oxazol-2-yl)-71)-8-(methylamino)-2,7-naphthyridin-3-yl)cyclopropanecarboxamide and 3-(2,2-difluoroethyl)azetidine hydrochloride as the starting material. LCMS (ESI) m/z 479.2, [M+H]⁺. ¹H NMR (400 MHz, DMSO-d₆) 11.02 (s, 1H), 9.65 (5, 1H), 9.43 (s, 1H), 8.87 (s, 1H), 8.72 8.47 (m, 1H), 7.52 (d, J=8.4 Hz, 1H), 6.69 (d, J=2.0 Hz, 1H), 6.62-6.40 (m, 1H), 6.37-5.90 (m, 1H), 4.10-4.00 (m, 2H), 3.61-3.52 (m, 2H), 3.09 (d, J=4.4 Hz, 3H), 3.00-2.90 (m, 1H), 2.30-2.18 (m, 2H), 2.16-2.04 (m, 1H), 0.98-0.79 (m, 4H).

### Example 320: Synthesis of (S)—N-(5-(5-(2-cyanoazetidin-1-yl)benzo[d]oxazol-2-yl)-8-(methylamino)-2,7-naphthyridin-3-3(1)cyclopropanecarboxamide

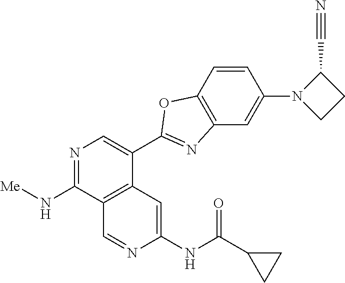 [SMILES: CNc1ncc(-c2nc3cc(N4CC[C@H]4C#N)ccc3o2)c2cc(NC(=O)C3CC3)ncc12]

### Step 1: (S)-azetidine-2-carbonitrile 2,2,2-trifluoroacetate

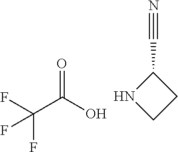

A solution of tert-butyl (S)-2-cyanoazetidine-1-carboxylate (50.0 mg, 0.274 mmol, 1.00 eq.) in a CH₂Cl₂/TFA (5:1, 4.8 mL) was stirred at room temperature for 1 hour under nitrogen atmosphere. The desired product was detected via LCMS. The resulting mixture was concentrated under vacuum to afford (S)-azetidine-2-carbonitrile 2,2,2-trifluoroacetate as white oil (81.0 mg, crude), LCMS (ESI) m/z 83.1, [M+H]⁺.

### Step 2: (S)—N-(5-(5-(2-cyanoazetidin-1-yl)benzo[d]oxazol-2-yl)-8-(methylamino)-2,7-naphthyridin-3-yl)cyclopropanecarboxamide

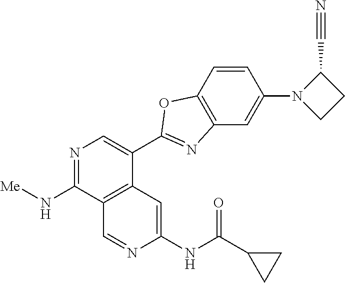 [SMILES: CNc1ncc(-c2nc3cc(N4CC[C@H]4C#N)ccc3o2)c2cc(NC(=O)C3CC3)ncc12]

(S)—N-(5-(5-(2-cyanoazetidin-1-yl)benzo[d]oxazol-2-yl)-8-(methylamino)-2,7-naphthyridin-3-yl)cyclopropanecarboxamide was synthesized using a similar procedure that was previously described in Example 289, by using N-(5-(5-bromobenzo[d]oxazol-2-yl)-8-(methylamino)-2,7-naphthyridin-3-yl)cyclopropanecarboxamide and (S)-azetidine-2-carbonitrile 2,2,2-trifluoroacetate as the starting material. LCMS (ESI) m/z 440.2, [M+H]⁺. ¹H NMR (400 MHz, Methanol-d₄) δ 9.76 (s, 1H), 9.28 (s, 1H), 8.91 (s, 1H), 7.59 (d, =8.8 Hz, 1H), 7.03 (d, J=2.4 Hz, 1H), 6.69 (dd, J=8.7, 2.4 Hz, 1H), 4.88-4.82 (m, 1H), 4.16-4.07 (m, 1H), 3.93-3.83 (m, 1H), 3.18 (s, 3H), 2.84-2.74 (m, 2H), 2.06-1.95 (m, 1H), 1.15-0.95 (m, 4H).

### Example 321: Synthesis of (S)-1-(2-(6-(cyclopropanecarboxamido)-1-(methylamino)-2,7-naphthyridin-4-yl)benzo[d]oxazol-5-1)azetidine-2-carboxamide

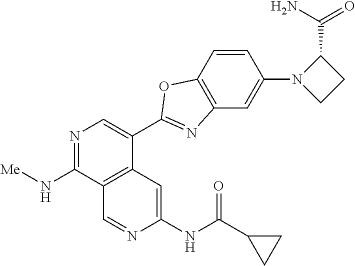 [SMILES: CNc1ncc(-c2nc3cc(N4CC[C@H]4C(N)=O)ccc3o2)c2cc(NC(=O)C3CC3)ncc12]

### Step 1: (S)-azetidine-2-carbonitrile hydrochloride

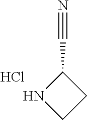

To a stirred solution of tert-butyl (9-2-cyanoazetidine-1-carboxylate (200.0 mg; 1.098 mmol; 1.00 eq.) in MeOH (8 mL) was added a solution of HCl in dioxane (4 N, 4 mL). The resulting solution was stirred for at room temperature 2 hours. The desired product was detected via LCMS. The resulting mixture was concentrated under vacuum to afford (S)-azetidine-2-carbonitrile hydrochloride as a white solid (194.0 mg, crude), LCMS (ESI) m/z 83.1, [M+H]⁺.

### Step 2: (8)-1-(2-(6-(cyclopropanecarboxamido)-1-(methylamino)-2,7-naphthyridin-4-yl)benzo[d]oxazol-5-yl)azetidine-2-carboxamide

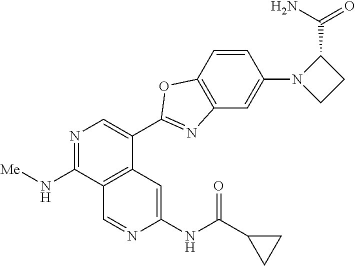 [SMILES: CNc1ncc(-c2nc3cc(N4CC[C@H]4C(N)=O)ccc3o2)c2cc(NC(=O)C3CC3)ncc12]

(S)-1-(2-(6-(cyclopropanecarboxamido)-1-(methylamino)-2,7-naphthyridin-4-yl)benzo[d]oxazol-5-yl)azetidine-2-carboxamide was synthesized using a similar procedure that was previously described in Example 277, by using N-(5-(5-bromobenzo[d]oxazol-2-yl)-8-(methylamino)-2,7-naphthyridin-3-yl)cyclopropanecarboxamide and (S)-azetidine-2-carbonitrile hydrochloride as the starting material. LCMS (ESI) m/z 458.2, [M+H]⁺. ¹H NMR (400 MHz, DMSO-d₄) δ 11.03 (s, 111), 9.67 (s, 1H), 9.43 (s, 1H), 8.89 (s, 1H), 8.67-8.60 (m, 1H), 7.63-7.55 (m, 2H), 7.29 (s, 1H), 6.70 (d, J=2.0 Hz, 1H), 6.48 (dd, J=8.4, 2.0 Hz, 1H), 4.23 (t, J=8.0 Hz, 1H), 4.02-3.93 (m, 1H), 3.74-3.64 (m, 1H), 3.08 (d, J=4.4 Hz, 3H), 2.60-2.55 (m, 1H), 2.43-2.32 (m, 1H), 2.15-2.05 (m, 1H), 0.94-0.82 (m, 4H).

### Example 322: Synthesis of N-(5-(5-(6-acetyl-2,6-diazaspiro[3.3]heptan-2-yl)benzo[d]oxazol-2-yl)-8-(methylamino)-2,7-naphthyridin-3-yl)cyclopropanecarboxamide

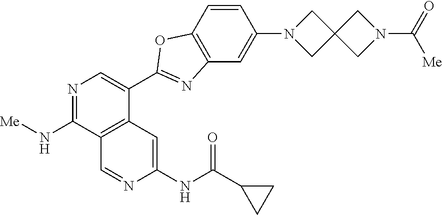 [SMILES: CNc1ncc(-c2nc3cc(N4CC5(CN(C(C)=O)C5)C4)ccc3o2)c2cc(NC(=O)C3CC3)ncc12]

### Step 1: left-butyl 6-acetyl-2,6-diazaspiro[3.3]heptane-2-carboxylate

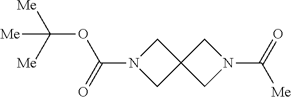 [SMILES: CC(=O)N1CC2(C1)CN(C(=O)OC(C)(C)C)C2]

To a solution of tell-butyl 2,6-diazaspiro[3.3]heptane-2-carboxylate (300.0 mg; 1.513 mmol; 1.00 eq.), DIEA (1172.7 mg; 9.073 mmol; 600 eq.) in CH₂Cl₂ (15 mL) was added acetic anhydride (309.0 mg; 3.027 mmol; 2.00 eq.) in portions at room temperature. The resulting mixture was stirred at room temperature overnight under nitrogen atmosphere. The desired product was detected via LCMS. The solution was quenched with water (10 mL), extracted with EtOAc (3×50 mL). The combined organic layers were washed with brine (3×10 mL), dried over anhydrous Na₂SO₄, filtered, and concentrated under reduced pressure. The residue was purified by flash chromatography on silica gel column using 10-30% of EtOAc in petroleum ether as eluent to provide tert-butyl 6-acetyl-2,6-diazaspiro[3.3]heptane-2-carboxylate as a white liquid (260 mg, 75.9° M. LCMS (ESI) m/z 241.1, [M+H]⁺.

### Step 2: 1-(2,6-diazaspiro[3:3]heptan-2-yl)ethan-1-one hydrochloride

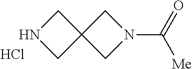

To a solution of tort-butyl 6-acetyl-2,6-diazaspiro[3.3]heptane-2-carboxylate (200.0 mg; 0.832 mmol; 1.00 eq.) in MeOH (1 mL) was added HCl (gas) (4 M in 1,4-dioxane, 5 mL). The resulting mixture was stirred at room temperature for 1 hour under nitrogen atmosphere. LC-MS indicated that the starting material was consumed. The solvent was concentrated under vacuum to afford 1-(2,6-diazaspiro[3.3]heptan-2-yl)ethan-1-one hydrochloride as a white solid (135.0 mg, 91.8%). LCMS (ESI) m/z 141.1, [M+H]⁺.

### Step 3: N-(5-(5-(6-acetyl-2,6-diazaspiro[3.3]heptan-2-11)benzo[d]oxazol-2-yl)-8-(methylamino)-2,7-naphthyridin-3-yl)cyclopropanecarboxamide

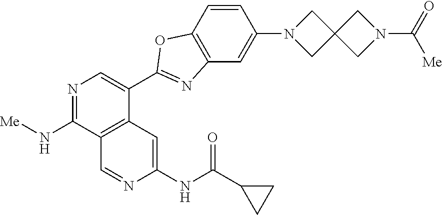 [SMILES: CNc1ncc(-c2nc3cc(N4CC5(CN(C(C)=O)C5)C4)ccc3o2)c2cc(NC(=O)C3CC3)ncc12]

N-(5-(5-(6-acetyl-2,6-diazaspiro[3.3]heptan-2-yl)benzo[d]oxazol-2-yl)-8-(methylamino)-2,7-naphthyridin-3-yl)cyclopropanecarboxamide as synthesized using a similar procedure that was previously described in Example 277 by using N-(5-(5-bromobenzo[d]oxazol-2-yl)-8-(methylamino)-2,7-naphthyridin-3-yl)cyopropanecarboxamide and 1-(2,6-diazaspiro[3.3]heptan-2-yl)ethan-1-one hydrochloride as the starting material. LCMS (ESI) m/z 498.2, [M+H]⁺. ¹H NMR (400 MHz, DMSO-d₆) δ 11.03 (s, 1H), 9.65 (s, 1H), 9.43 (s, 1H), 8.87 (s, 1H), 8.67_8.60 (m, 1H), 7.54 (d, J=8.8 Hz, 1H), 6.73 (d, J=2.4 Hz, 1H), 6.52-6.66 (m, 1H), 4.32 (s, 2H), 4.09-4.02 (m, 6H), 3.08 (d, J=4.4 Hz, 3H), 2.14-2.06 (m, 1H), 1.77 (s, 3H), 0.98-0.81 (m, 4H).

### Example 323: Synthesis of N-(5-(5-(3-(2-cyanoethoxy)azetidin-1-yl)benzo[d]oxazol-2-yl)-8-(methylamino)-2,7-naphthyridin-3-yl)cyclopropanecarboxamide

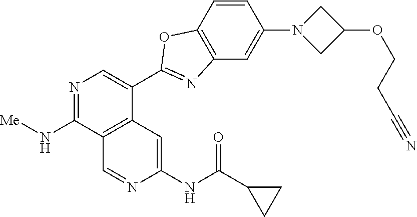 [SMILES: CNc1ncc(-c2nc3cc(N4CC(OCCC#N)C4)ccc3o2)c2cc(NC(=O)C3CC3)ncc12]

### Step 1: tert-butyl 3-(2-cyanoethoxy)azetidine-1-carboxylate

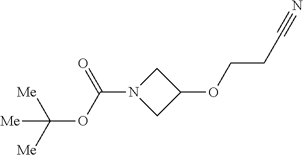 [SMILES: CC(C)(C)OC(=O)N1CC(OCCC#N)C1]

A solution of tert-butyl 3-hydroxyazetidine-1-carboxylate (1.00 g; 5.773 mmol; 1.00 eq.), K₂CO₃(79.8 mg; 0.577 mmol; 0.10 eq.) and t-BuOH (42.8 mg; 0.577 mmol; 0.10 eq.) in acrylonitrile (10 mL) was stirred at 80° C. for 2 hours under nitrogen atmosphere. The desired product was detected vis LCMS. The resulting mixture was concentrated under vacuum. The residue was dissolved in CH₂Cl₂ (200 mL) and washed with saturated sodium chloride solution (3×10 mL), the organic layer was dried over anhydrous Na₂SO₄. After filtration, the filtrate was concentrated under reduced pressure to provide tert-butyl 3-(2-cyanoethoxy)azetidine-1-carboxylate a yellow solid (1.10 g, 84.2%). LCMS (ESI) m/z 227.1, [M+H]⁺.

### Step 2: 3-(azetidin-3-yloxy)propanenitrile hydrochloride

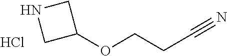

To a stirred solution of tert-butyl 3-(2-cyanoethoxy)azetidine-1-carboxylate (200.0 mg; 0.884 mmol; 1.00 eq.) in CH₂Cl₂ (O 0.5 mL) was added a solution of HCl in ether (2 M, 2 mL) dropwise at 0° C. under nitrogen atmosphere. The resulting mixture was stirred at room temperature for 3 hours. The resulting mixture was concentrated under vacuum to afford 3-(azetidin-3-yloxy)propanenitrile hydrochloride as a yellow solid (120 mg, crude). LCMS (ESI) m/z 127.1, [M+H]⁺.

### Step 3: N-(5-(5-(3-(2-cyanoethoxy)azetidin-1-yl)benzo[d]oxazol-2-yl)-8-(methylamino)-2,7-naphthyridin-3-yl)cyclopropanecarboxamide

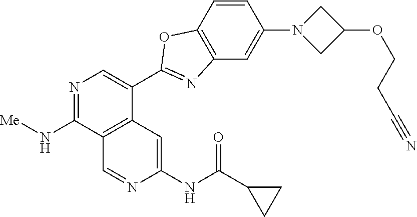 [SMILES: CNc1ncc(-c2nc3cc(N4CC(OCCC#N)C4)ccc3o2)c2cc(NC(=O)C3CC3)ncc12]

N-(5-(5-(3-(2-cyanoethoxy)azetidin-1-yl)benzo[d]oxazol-2-yl)-8-(methy amino)-2,7-naphthyridin-3-yl)cyclopropanecarboxamide was synthesized using a similar procedure that was previously described in Example 289, by using N-(5-(5-bromobenzo[d]oxazol-2-yl)-8-(methylamino)-2,7-naphthyridin-3-yl)cyclopropanecarboxamide and 3-(azetidin-3 yloxy)propanenitrile hydrochloride as the starting material. LCMS (ESI) m/z 484.2, [M+H]⁺. NMR (400 MHz, DMSO-d₆) δ 11.02 (s, 1H), 9.66 (s, 1H), 9.43 (s, 1H), 8.88 (s, 1H), 8.66-8.58 (m, 1H), 7.54 (d, J=8.4 Hz, 1H), 6.74 (d, J=1.2 Hz, 1H), 6.54-6.47 (m, 1H), 4.57-4.49 (m, 1H, 4.15 (d, J=6.4 Hz, 2H), 3.72-3.60 (m, 4H), 3.09 (d, J=4.4 Hz, 3H), 2.82 (d, J=6.4 Hz, 2H), 2.14-2.07 (m, 1H), 0.95-0.89 (m, 2H), 0.89-0.82 (m, 2H).

### Example 324: Synthesis of N-(5-(5-(3-(2-methoxyethoxy)azetidin-1-yl)benzo[d]oxazol-2-yl)-8-(methylamino)-2,7-naphthyridin-3-yl)cyclopropanecarboxamide

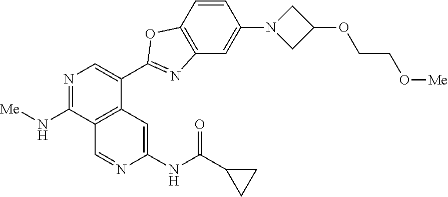 [SMILES: CNc1ncc(-c2nc3cc(N4CC(OCCOC)C4)ccc3o2)c2cc(NC(=O)C3CC3)ncc12]

### Step 1: tert-butyl 3-(2-methoxyethoxy)azetidine-1-carboxylate

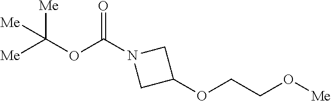 [SMILES: COCCOC1CN(C(=O)OC(C)(C)C)C1]

To a stirred solution of tert-butyl 3-hydroxyazetidine-1-carboxylate (2.00 g; 11.547 mmol; 1.00 eq.) and Cs₂CO₃ (1.12 g; 34.640 mmol; 3.00 eq.) in DMF (5 mL) was added 2-bromoethyl methyl ether (11 mL; 57.733 mmol; 5.00 eq.) dropwise at room temperature under nitrogen atmosphere. The resulting mixture was stirred at room temperature for 14 hours. The resulting mixture was concentrated under vacuum. The residue was purified by flash chromatography on silica gel column using 0-50% of EtOAc in petroleum ether as eluent to provide ten-butyl 3-(2-methoxyethoxy)azetidine-1-carboxylate as a colorless oil. (2.00 g, 74.8%) LCMS (ESI) m/z 232.1, [M+H]⁺.

### Step 2: 3-(2-ethoxyethoxy)azetidine hydrochloride

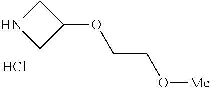

To a stirred solution of tert-butyl 3-(2-methoxyethoxy)azetidine-1-carboxylate (500.0 mg; 2.162 mmol; 1.00 eq.) in MeOH (5 mL) was added. HCl (gas) (l Min 1,4-dioxane, 25 mL) dropwise at 0° C. under nitrogen atmosphere. The resulting mixture was stirred at room temperature for 1 hour. The resulting mixture was concentrated under vacuum to afford 3-(2-methoxyethoxy)azetidine hydrochloride as a yellow solid (320 mg, crude). LCMS (ESI) m/z 132.1, [M+H]⁺.

### Step 3: N-(5-(5-(3-(2-methoxyethoxy)azetidin-1-yl)benzo[d]oxazol-2-yl)-8-(methylamino)-2,7-naphthyridin-3-yl)cyclopropanecarboxamide

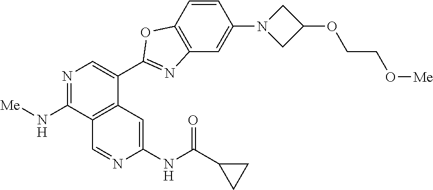 [SMILES: CNc1ncc(-c2nc3cc(N4CC(OCCOC)C4)ccc3o2)c2cc(NC(=O)C3CC3)ncc12]

N-(5-(5-(3-(2-methoxyethoxy)azetidin-1-yl)benzo[d]oxazol-2-yl)-8-(methylamino)-2,7-naphthyridin-3-yl)cyclopropanecarboxamide was synthesized using a similar procedure that was previously described in Example 289, by using N-(5-(5-bromobenzo[d]oxazol-2-yl)-8-(methylamino)-2,7-naphthyridin-3-yl)cyclopropanecarboxamide and 3-(2-methoxyethoxy)azetidine hydrochloride as the starting material. LCMS (ESI) m/z 489.2, [M+H]⁺. ¹H NMR (400 MHz, DMSO-d₆) δ 11.06 (s, 1H), 9.67 (5, 1H), 9.45 (s, 1H), 8.85 (s, 1H), 8.80-8.52 (m, 1H), 7.54 (d, J=8.4 Hz, 1H), 6.72 (d, J=1.6 Hz, 1H), 6.66-6.30 (m, 1H), 4.52-4.35 (m, 1H), 4.21-4.05 (m, 2H), 3.68-3.61 (m, 2H), 3.59-3.53 (m, 2H), 3.51-3.47 (m, 2H), 3.28 (s, 3H), 3.09 (d, J=4.4 Hz, 3H), 2.16-2.02 (m, 1H), 1.00-0.80 (m, 4H).

### Example Synthesis of N-(5-(5-(7-methyl-3,7-diazabicyclo[3.3.1]nonan-3-yl)benzo[d]oxazol-2-yl)-8-(methylamino)-2,7-naphthyridin-3-yl)cyclopropanecarboxamide

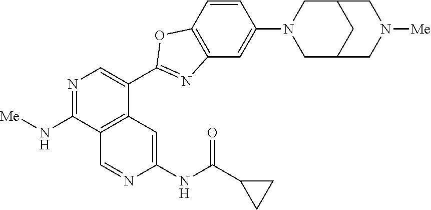 [SMILES: CNc1ncc(-c2nc3cc(N4CC5CC(CN(C)C5)C4)ccc3o2)c2cc(NC(=O)C3CC3)ncc12]

### Step 1: tert-butyl 7-(2-(6-(cyclopropanecarboxamido)-1-methylamino yl)benzo[d]oxazol-5-yl)-3,7-diazabicyclo[3.3.1]nonane-3-carboxylate

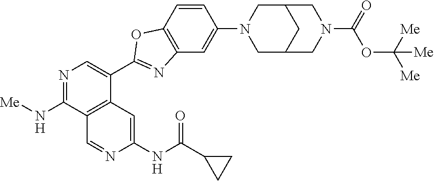 [SMILES: CNc1ncc(-c2nc3cc(N4CC5CC(CN(C(=O)OC(C)(C)C)C5)C4)ccc3o2)c2cc(NC(=O)C3CC3)ncc12]

A suspension of N-(5-(5-bromobenzo[d]oxazol-2-yl)-8-(methylamino)-2,7-n yl)cyclopropanecarboxamide (200.0 mg; 0.456 mmol; 1.00 eq.), tert-butyl 3,7-diazabicyclo[3.3.1]nonane-3-carboxylate (309.8 mg; 1.369 mmol; 3.00 eq.), EPhos (48.8 mg; 0.091 mmol; 0.20 eq.) and EPhos Pd G₄ (83.8 mg; 0.091 mmol; 0.20 eq.) and Cs₂CO₃ (746.4 mg; 2.291 mmol; 5.02 eq.) in 1,4-dioxane (10 mL) was stirred at 100° C. for 24 hours under nitrogen atmosphere. The desired product was detected via. LCMS. The resulting mixture was cooled to room temperature and concentrated under reduced pressure. The residue was purified by flash chromatography on silica gel column using 0-10% of MeOH in CH₂Cl₂ as eluent to provide the crude product. The residue was purified by flash chromatography on pre-packed C18 column using 30-100% of MeOH in water (10 mmol/L NH₄HCO₃) as eluent to provide tert-butyl 7-(2-(6-(cyclopropanecarboxamido)-1-(methyl amino)-2,7-naphthyridin-4-yl)benzo[d]oxazol-5-yl)-3,7-diazabicyclo[3.3.1]nonane-3-carboxylate as a yellow solid (96.0 mg, 34.2%). LCMS (ESI) m/z 584.3, [M+H]⁺.

### Step 2: N-(5-(5-(3,7-diazabicyclo[3.3.1]nonan-3-yl)benzo[d]oxazol-yl)-8-(methylamino 2,7-naphthyridin-3-yl)cyclopropanecarboxamide hydrochloride

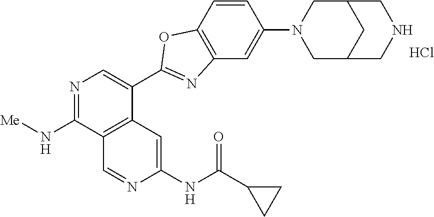 [SMILES: CNc1ncc(-c2nc3cc(N4CC5CNCC(C5)C4)ccc3o2)c2cc(NC(=O)C3CC3)ncc12 ; Cl]

To a stirred solution of tert-butyl 7-(2-(6-(cyclopropanecarboxamido)-1-(methylamino)-2,7-naphthyridin-4-yl)benzo[d]oxazol-5-yl)-3,7-diazabicyclo[3.3.1]nonane-3-carboxylate (100.0 mg; 0.170 mmol; 1.00 eq.) in MeOH (7.5 mL) was added HCl (gas) (4 M in 1,4-dioxane, 22.5 mL) at 0° C. under nitrogen atmosphere. The resulting mixture was stirred at room temperature for 3 hours. The resulting mixture was concentrated under vacuum to afford N-(5-(5-(3,7-diazabicyclo[3.3.1]nonan-3-yl)benzo[d]oxazol-2-yl)-8-(methylamino)-2,7-naphthyridin-3-yl)cyclopropanecarboxamide hydrochloride as a yellow solid (95 mg, crude), LCMS (ESI) m/z 484.2, [M+H]⁺.

### Step 3: N-(5-(5-(7-methyl-3,7-diazabicyclo[3.3.1]nonan-3-yl)benzo[d]oxazol-2-yl)-8-(methyl amino)-2,7-naphthyridin-3-yl)cyclopropanecarboxamide

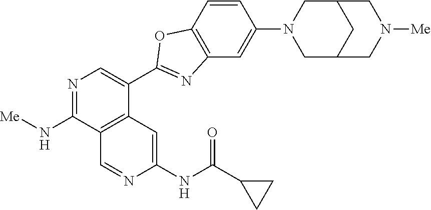 [SMILES: CNc1ncc(-c2nc3cc(N4CC5CC(CN(C)C5)C4)ccc3o2)c2cc(NC(=O)C3CC3)ncc12]

To a stirred solution of N-(5-(5-(3,7-diazabicyclo[3.3.1]nonan-3-yl)benzo[d]oxazol-2-yl)-8-(methyl amino)-2,7-naphthyridin-3-yl)cyclopropanecarboxamide hydrochloride (95.0 mg; 0.183 mmol; 1.00 eq.) and HCHO (37% w/w in water) (44.5 mg; 0.548 mmol; 3.00 eq.) in CH₂Cl₂ (18 mL) was DIPEA (236.1 mg; 1.827 mmol; 10.00 eq.) dropwise at room temperature. The resulting mixture was stirred at room temperature for 2 hours. To the above mixture was added NaBH(OAc)₃ (116.2 mg; 0.548 mmol; 3.00 eq.) in portions. The resulting mixture was stirred at room temperature for 14 hours. The resulting mixture was concentrated under reduced pressure. The residue was purified by flash chromatography on silica gel column using 0-10% of MeOH in CH₂Cl₂ as eluent to provide N-(5-(5-(7-methyl-3,7-diazabicyclo[3.3.1]nonan-3-yl)benzo[d]oxazol-2-yl)-8-(methylamino)-2,7-naphthyridin-3-yl)cyclopropanecarboxamide as a yellow solid (24.6 mg, 26.7%). LCMS (ESI) m/z 498.3, [M+H]⁺. ¹H NMR (400 MHz, DMSO-d₆) 11.03 (s, 1H), 9.69 (s, 1H), 9.43 (s, 1H), 8.89 (s, 1H), 8.65-8.53 (m, 1H), 7.52 (d, J=8.8 Hz, 1H), 6.95 (d, J=2.4 Hz, 1H), 6.89-6.80 (m, 1H), 3.63 (d, J=11.2 Hz, 2H), 3.14-3.06 (m, 5H), 2.94-2.85 (m, 2H), 2.20-2.05 (m, 8H), 1.72-1.65 (m, 1H), 1.60-1.52 (m, 1H), 0.96-0.83 (m, 4H).

### Example 326: Synthesis of N-(5-(5-(7-acetyl-3,7-diazabicyclo[3.3.1]nonan-3-yl)benzo[d]oxazol-2-yl)-8-(methylamino)-2,7-naphthyridin-3-yl)cyclopropanecarboxamide

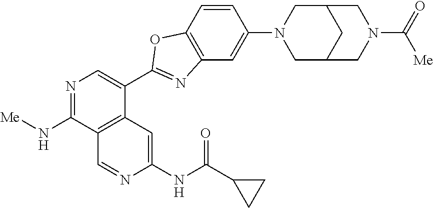 [SMILES: CNc1ncc(-c2nc3cc(N4CC5CC(CN(C(C)=O)C5)C4)ccc3o2)c2cc(NC(=O)C3CC3)ncc12]

A solution of N-(5-(5-(3,7-diazabicyclo[3.3.1]nonan-3-yl)benzo[d]oxazol-2-yl)-8-(methylamino)-2,7-naphthyridin-3-yl)cyclopropanecarboxamide hydrochloride (Example 325, step 2) (40.0 mg; 0.077 mmol; 1.00 eq.), acetic anhydride (12.6 mg; 0.123 mmol; 1.60 eq.) and DIEA (70.1 mg; 0.542 mmol; 7.05 eq.) in CH₂Cl₂(5 mL) was stirred at room temperature for 5 hours under nitrogen atmosphere. The desired product was detected via LCMS. The resulting mixture was concentrated under reduced pressure. The residue was purified by flash chromatography on pre-packed C18 column using 20-60% of MeCN in water (10 mmol/L NH₄HCO₃) to provide N-(5-(5-(7-acetyl-3,7-diazabicyclo[3.3.1]nonan-3-yl)benzo[d]oxazol-2-yl)-8-(methylamino)-2,7-naphthyridin-3-yl)cyclopropanecarboxamide as a yellow solid (13.1 mg, 32.4%). LCMS (ESI) m/z 526.2, [M+H]⁺. ¹H NMR (400 MHz, DMSO-d₆) δ 11.01 (s, 1H), 9.64 (s, 1H), 9.43 (s, 1H), 8.87 (s, 1H), 8.64-8.53 (m, 1H), 7.52 (d, J=8.8 Hz, 1H), 7.09 (d, J=2.4 Hz, 1H), 6.95 (dd, J=8.8, 2.4 Hz, 1H), 4.69-4.60 (m, 1H), 4.09-3.99 (m, 1H), 3.94-3.86 (m, 1H), 3.74-3.65 (m, 1H), 3.42-3.32 (m, 1H), 3.09 (d, J=4.4 Hz, 3H), 3.02-2.89 (m, 2H), 2.87-2.68 (m, 1H), 2.18-2.00 (m, 3H), 1.92-1.80 (m, 4H), 1.81-1.72 (m, 1H), 0.92-0.87 (m, 41).

### Example 327: Synthesis of N-(5-(5-(7-methyl-9-oxa-3,7-diazabicyclo[3.3.1]nonan-3-yl)benzo[d]oxazol-2-yl)-8-(methylamino)-2,7-naphthyridin-3-yl)cyclopropanecarboxamide

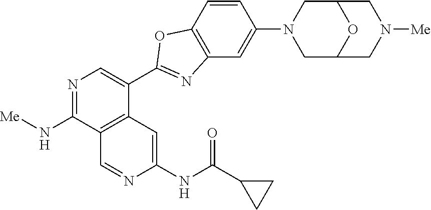 [SMILES: CNc1ncc(-c2nc3cc(N4CC5CN(C)CC(C4)O5)ccc3o2)c2cc(NC(=O)C3CC3)ncc12]

### Step 1: tert-butyl 7-(2-(6-(cyclopropanecarboxamido)-1-(methylamino)-2,7-naphthyridin-4-yl)benzo[d]oxazol-5-yl)-9-oxa-3,7-diazabicyclo[3.3.1]nonane-3-carboxylate

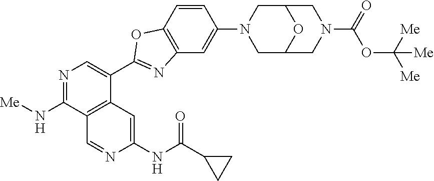 [SMILES: CNc1ncc(-c2nc3cc(N4CC5CN(C(=O)OC(C)(C)C)CC(C4)O5)ccc3o2)c2cc(NC(=O)C3CC3)ncc12]

A mixture of N-(5-(5-bromobenzo[d]oxazol-2-yl)-8-(methylamino)-2,7-naphthyridin-3-yl)cyclopropanecarboxamide (200.0 mg; 0.456 mmol; 1.00 eq.), tert-butyl 9-oxa-3,7-diazabicyclo[3.3.1]nonane-3-carboxylate (312.5 mg; 1.369 mmol; 3.00 eq.), EPhos Pd (14 (125.8 mg; 0.137 mmol; 0.30 eq.), EPhos (73.2 mg; 0.137 mmol; 0.30 eq.) and Cs₂CO₃ (446.1 mg; 1.369 mmol; 3.00 eq.) in 1,4-dioxane (12 mL) was stirred at 100° C. for 48 hours under nitrogen atmosphere. The desired product was detected via LCMS. The resulting mixture was concentrated under reduced pressure. The residue was purified by flash chromatography on silica gel column using 5-10% of MeOH in CH₂Cl₂ as eluent to afford tert-butyl 7-(2-(6-cyclopropanecarboxamido)-1-(methylamino)-2,7-naphthyridin-4-yl)benzo[d]oxazol-5-yl)-9-oxa-3,7-diazabicyclo[33.1]nonane-3-carboxylate as a yellow solid (200.0 mg, 67.4%). LCMS (ESI) m/z 586.3, [M+H]⁺.

### Step 2: N-(5-(5-(9-oxa-3,7-diazabicyclo[3.3.1]nonan-3-yl)benzo[d]oxazol-2-yl)-8-(methylami no)-2,7-naphthyridin-3-yl)cyclopropanecarboxamide hydrochloride

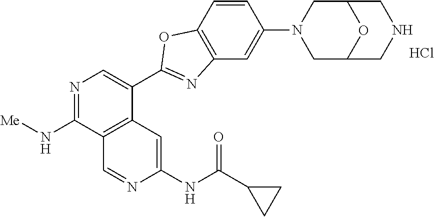 [SMILES: CNc1ncc(-c2nc3cc(N4CC5CNCC(C4)O5)ccc3o2)c2cc(NC(=O)C3CC3)ncc12 ; Cl]

A mixture of tert-butyl 7-(2-(6-(cyclopropanecarboxamido)-1-(methylamino)-2, 7-naphthyridin-4-yl)benzo[d]oxazol-5-yl)-9-oxa-3,7-diazabicyclo[3.3.1]nonane-3-carboxylate (200.0 mg; 0.341 mmol; 1.00 eq.) in CH₂Cl₂ (20 mL) was added a solution of HCl in MeOH (4N, 5 mL). The resulting solution was stirred at room temperature for 2 hours under nitrogen atmosphere. LC-MS indicated that the reaction was complete. The solvent was concentrated under vacuum to afford N-(5-(5-(9-oxa-3,7-diazabicyclo[3.3.1]nonan-3-yl)benzo[d]oxazol-2-yl)-8-(methylamino)-2,7-naphthyridin-3-yl)cyclopropanecarboxamide hydrochloride (150.0 mg, 79.9%). LCMS (ESI) m/z 486.2, [M+H]⁺.

### Step 3: N-(5-(5-(7-methyl-9-oxa-3,7-diazabicyclo[3.3.1]nonan-3-yl)benzo[d]oxazol-2-yl)-8-(methylamino)-2,7-naphthyridin-3-yl)cyclopropanecarboxamide

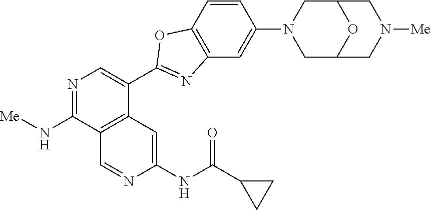 [SMILES: CNc1ncc(-c2nc3cc(N4CC5CN(C)CC(C4)O5)ccc3o2)c2cc(NC(=O)C3CC3)ncc12]

A mixture of N-(5-(5-(9-oxa-3,7-diazabicyclo[3.3.1]nonan-3-yl)benzo[d]oxazol-2-yl)-8-(methylamino)-2,7-naphthyridin-3-yl)cyclopropanecarboxamide hydrochloride (120.0 mg; 0.247 mmol; 1.00 eq.) and HCHO (37% w/w in water, 37.1 mg) in CH₂Cl₂ (10 mL) was stirred at room temperature for 0.5 hour. To the above solution was added NaBH(OAc)₃ (157.2 mg; 0.742 mmol; 3.00 eq.). The resulting solution was stirred at room temperature for an additional of 2 hours. The desired product was detected via LCMS. The resulting mixture was concentrated under reduced pressure. The residue was purified by flash chromatography on silica gel column using 5-10% of MeOH in CH₂Cl₂ where CH₂Cl₂ (0.1% triethylamine) as eluent to afford a crude product. The residue was purified by preparative Achiral-SFC with the following conditions (Column: GreenSep Basic 3×15 cm, 5 μm; Mobile Phase A: CO₂, Mobile Phase B: MeOH (0.1% 2 M NH₃-MeOH); Flow rate: 75 mL/rain; Gradient: isocratic 50% B; Wave Length: 254 nm; R_{T}(min): 2.47) to afford 3-(5-(5-(7-methyl-9-oxa-3,7-diazabicyclo[3.3.1]nonan-3-yl)benzo[d]oxazol-2-yl)-8-(methylamino)-2,7-naphthyridin-3-yl)cyclopropanecarboxamide as a yellow solid (44.1 mg, 35.4%). LCMS (ESI) m/z 500.2, [M+H]⁺. ¹H NMR, (400 MHz, DMSO-d₆) δ 11.03 (s, 1H), 9.69 (s, 1H), 9.45 (s, 1H), 8.90 (s, 1H), 8.72-8.52 (m, 1H), 7.59 (d, J=8.8 Hz, 1H), 7.20 (d, J=2.4 Hz, 1H), 7.00 (dd, =8.8, 2.4 Hz, 1H), 4.22-4.11 (s, 2H), 3.70 (d, J=11.6 Hz, 2H), 3.39-3.00 (m, 7H), 2.90-2.58 (m, 2R), 2.50-2.19 (m, 3H), 2.15-2.02 (m, 1H), 0.98-0.76 (m, 4H).

### Example 328: Synthesis of N-(5-(5-(1,1-dioxidoisothiazolidin-2-yl)benzo[d]oxazol-2-yl)-8-(methylamino)-2,7-naphthyridin-3-yl)cyclopropanecarboxamide

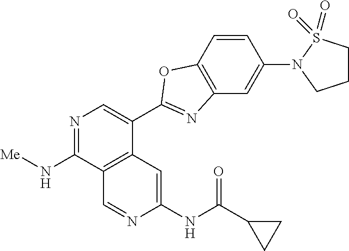 [SMILES: CNc1ncc(-c2nc3cc(N4CCCS4(=O)=O)ccc3o2)c2cc(NC(=O)C3CC3)ncc12]

A solution of N-(5-(5-bromobenzo[d]oxazol-2-yl)-8-(methylamino)-2,7-naphthyridin-3-yl)cyclopropanecarboxamide (50.0 mg; 0.114 mmol; LOU eq.) and isothiazolidine 1,1-dioxide (20.7 mg; 0.171 mmol; 1.50 eq.) in 1,4-dioxane mL). To the above solution were added t-BuXPhos Pd G₃ (9.0 mg; 0.011 mmol; 0.10 eq.), t-BuXPhos (4.8 mg; 0.011 mmol; 0.10 eq.) and Cs₂CO₃ (74.3 mg; 0.228 mmol; 2.00 eq.). The resulting solution was stirred at 90° C. for 14 hours under nitrogen atmosphere. The desired product was detected via LCMS. The resulting mixture was concentrated under reduced pressure. The residue was purified by flash chromatography on silica gel column using 5-10% of MeOH in CH₂Cl₂ as eluent to afford a crude product. The crude product was purified by flash chromatography on pre-packed. C18 column using 30-60% of MeOH in water (10 mmol/L NH₄HCO₃) as eluent to provide N-(5-(5-(1,1-dioxidoisothiazolidin-2-yl)benzo[d]oxazol-2-yl)-8-(methylamino)-2,7-naphthyridin-3-yl)cyclopropanecarboxamide as a yellow solid (43.4 mg, 73.8%). LCMS (ESP) m/z 479.1 [M+H]⁺. ¹H NMR, (400 MHz, DMSO-d₆) 1105 (s, 1H), 9.71 (s, 14), 9.45 (s, 1H), 8.93 (s, 8.75-8.65 (m, 1H), 7.75 (d, J=8.8 Hz, 1H), 7.57 (d, J=2.4 HZ, 1H), 7.32-7.25 (m, 1H), 3.91-3.82 (m, 2H), 3.60-3.53 (m, 2H), 3.10 (d, J=4.4 Hz, 3H), 2.49-2.41 (m, 2H), 2.16-2.08 (m, 1H), 0.98-0.91 (m, 2H), 0.90-0.82 (m, 2H).

### Example 329: Synthesis of N-(5-(5-(2-(difluoromethyl)morpholino)benzo[d]oxazol-2-yl)-8-(methylamino)-2,7-naphthyridin-3-yl)cyclopropanecarboxamide

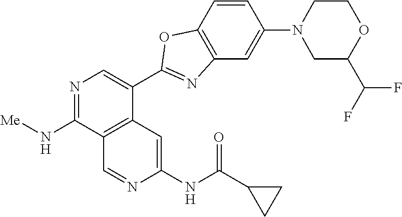 [SMILES: CNc1ncc(-c2nc3cc(N4CCOC(C(F)F)C4)ccc3o2)c2cc(NC(=O)C3CC3)ncc12]

### Step 1: tert-butyl 2-formylmorpholine-4-carboxylate

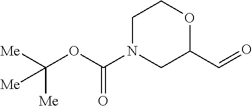 [SMILES: CC(C)(C)OC(=O)N1CCOC(C=O)C1]

To a solution of tert-butyl 2-(hydroxymethyl)morpholine-4-carboxylate (10.00 g; 46.027 mmol; 1.00 eq.) in CH₂Cl₂ (100 mL) was added Dess-Martin (23.40 g; 55.170 mmol; 1.20 eq.) in batches at 0° C. The resulting solution was stirred at 0° C. for 0.5 hour and at room temperature for addition 2 hours. The desired product was detected via TLC. The mixture was poured into a saturated sodium bicarbonate solution (100 mL). The organic phase was washed with brine (2×30 mL), dried over anhydrous sodium sulfate, and concentrated under reduced pressure to provide tert-butyl 2-formylmorpholine-4-carboxylate as a yellow oil (7.70 g, crude). The crude product was used in the next step without further purification.

### Step 2: tert-butyl 2-(difluoromethyl)morpholine-4-carboxylate

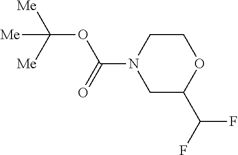 [SMILES: CC(C)(C)OC(=O)N1CCOC(C(F)F)C1]

To a solution of Teri-butyl 2-formylmorpholine-4-carboxylate (1.00 g; 4.646 mmol; 1.00 eq.) in dichloromethane (20 mL) was added diethylaminosulphurtrifluoride (1.72 g; 10.685 mmol; 2.30 eq.) at 0° C. The mixture was stirred at room temperature for 14 hours. The desired product was detected via LCMS. The reaction mixture was quenched with saturated NaHCO₃ solution (20 mL) and extracted with dichloromethane (3×30 mL). The combined organic layers were dried over anhydrous Na₂SO₄ and concentrated under reduced pressure. The residue was purified by flash chromatography on silica gel column using 10-50% of EtOAc in petroleum ether as eluent to provide Teri-butyl 2-(difluoromethyl)morpholine-4-carboxylate as a yellow oil (600.0 mg, 54.4%). LCMS (ESI) m/z 238.1, [M+H]⁺.

### Step 3: 2-(difluoromethyl)morpholine hydrochloride

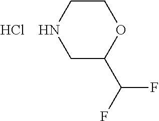

A solution of Teri-butyl 2-(difluoromethyl)morpholine-4-carboxylate (100.0 mg; 0.422 mmol; 1.00 eq.) in HCl (gas) (4 M in 1,4-dioxane, 3 mL) was stirred at room temperature for 1 hour. The resulting mixture was concentrated under reduced pressure to provide 2-(difluoromethyl)morpholine hydrochloride as a yellow oil (60.0 mg, 82.0%). LCMS (ESI) 138.1, [M+H]⁺.

### Step 4: N-(5-(5-(2-(difluoromethyl)morpholino)benzo[d]oxazol-2-yl)-8-(methylamino)-2,7-naphthyridin-3-yl)cyclopropanecarboxamide

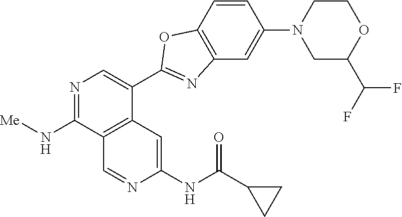 [SMILES: CNc1ncc(-c2nc3cc(N4CCOC(C(F)F)C4)ccc3o2)c2cc(NC(=O)C3CC3)ncc12]

N-(5-(5-(2-(difluoromethyl)morpholino)benzo[d]oxazol-2-yl)-8-(methy amino)-2,7-naphthyridin-3-yl)cyclopropanecarboxamide was synthesized using a similar procedure that was previously described in Example 289, by using N-(5-(5-bromobenzo[d]oxazol-2-yl)-8-(methylamino)-2,7-naphthyridin-3-yl)cyclopropanecarboxamide and (difluoromethyl)morpholine hydrogen chloride as the starting material. LCMS (ESI) m/z 495.2, [M+H]⁺. ¹H NMR (400 MHz, DMSO-d₆) δ 11.03 (s, 1H), 9.65 (s, 1H), 9.44 (s, 1H), 8.89 (s, 1H), 8.66-8.59 (m, 1H), 7.61 (d, 8.8 Hz, 1H), 7.28 (d, J=2.4 Hz, 1H), 7.10 (dd, J=8.8, 2.4 Hz, 1H), 6.30-5.98 (m, 1H), 4.11-3.89 (m, 2H), 3.81-3.72 (m, 1H), 3.67-3.49 (m, 2H), 3.09 (d, J=4.4 Hz, 3H), 2.87-2.65 (m, 2H), 2.14-2.07 (m, 1H), 0.94-0.81 (m, 4H).

### Example 330, Example 331, and Example 332: Synthesis of N-(5-(5-(2-(1,1-difluoroethyl)morpholino)benzo[d]oxazol-2-yl)-8-(methylamino)-2,7-naphthyridin-3-yl)cyclopropanecarboxamide (Example 330), (S)—N-(5-(5-(2-(1,1-difluoromethyl)morpholino)benzo[d]oxazol-2-yl)-8-(methylamino)-2,7-naphthyridin-3-yl)cyclopropanecarboxamide (331) and (R)—N-(5-(5-(2-(1,1-difluoromethyl)morpholino)benzo[d]oxazol-2-yl)-8-(methylamino)-2,7-naphthyridin-3-yl)cyclopropanecarboxamide (Example 332)

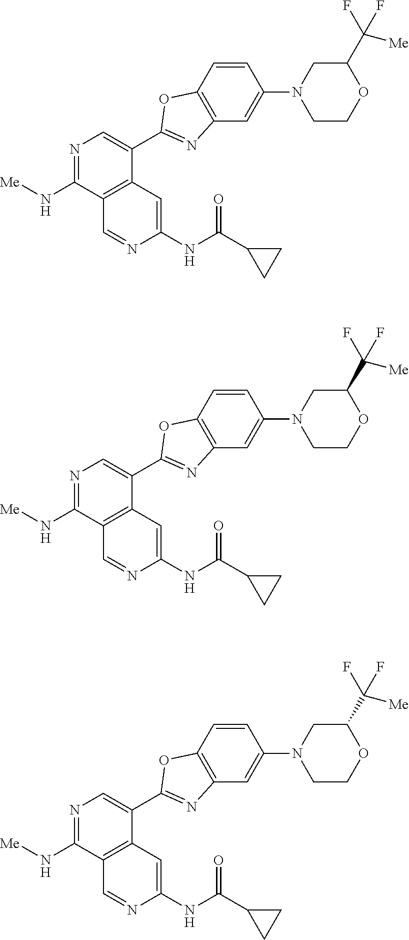 [SMILES: CNc1ncc(-c2nc3cc(N4CCOC(C(C)(F)F)C4)ccc3o2)c2cc(NC(=O)C3CC3)ncc12 ; CNc1ncc(-c2nc3cc(N4CCO[C@H](C(C)(F)F)C4)ccc3o2)c2cc(NC(=O)C3CC3)ncc12 ; CNc1ncc(-c2nc3cc(N4CCO[C@@H](C(C)(F)F)C4)ccc3o2)c2cc(NC(=O)C3CC3)ncc12]

### Step 1: text-butyl 2-(1,1-difluoroethyl)morpholine-4-carboxylate

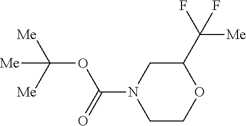 [SMILES: CC(C)(C)OC(=O)N1CCOC(C(C)(F)F)C1]

To a solution of tert-butyl 2-acetylmorpholine-4-carboxylate (250.0 mg; 1.090 mmol; 1.00 eq.) in CHCl₃ (3 mL) was added BAST (965.0 mg; 4.362 mmol; 4.00 eq.) dropwise at 0° C. under nitrogen atmosphere. The reaction was stirred at 60° C. for 16 hours. The desired product was detected via LCMS. The reaction was quenched with saturated NaHCO₃ solution at 0° c. The resulting mixture was extracted with EtOAc (2×50 mL). The combined organic layers were washed with brine (3×10 mL), dried over anhydrous Na₂SO₄, filtered, and concentrated under reduced pressure. The residue was purified by flash chromatography on silica gel column using 5-20% of EtOAc in petroleum ether as eluent to provide tert-butyl 2-(1,1-difluoroethyl)morpholine-4-carboxylate as a white soild (198 mg, 72.2%), LCMS (ESI) m/z 252.1, [M+H]⁺.

### Step 2: 2-(1,1-difluoroethyl)morpholine hydrochloride

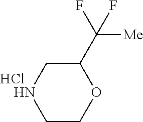

To a solution of tert-butyl 2-(1,1-difluoroethyl)morpholine-4-carboxylate (198.0 mg; 0.788 mmol, 1.00 eq.) in EtOAc (1 mL) was added a solution of HCl in EtOAc (4 N, 5 nit) at 0° C. The reaction was stirred at room temperature for 1 hour. The mixture was concentrated under vacuum to afford 2-(1,1-difluoroethyl)morpholine hydrochloride as a white solid (139.0 mg, crude). LCMS (ESI) m/z 152.1, [M+H]⁺.

### Step 3: N-(5-(5-(2-(1,1-difluoroethyl)morpholino)benzo[d]oxazol-2-yl)-8-(methylamino)-2,7-naphthyridin-3-yl)cyclopropanecarboxamide

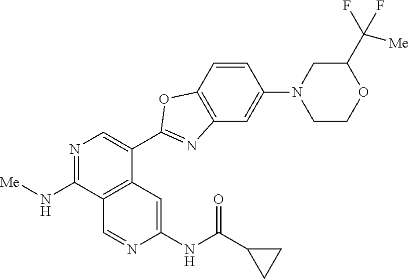 [SMILES: CNc1ncc(-c2nc3cc(N4CCOC(C(C)(F)F)C4)ccc3o2)c2cc(NC(=O)C3CC3)ncc12]

N-(5-(5-(2-(1,1-difluoroethyl)morpholino)benzo[d]oxazol-2-yl)-8-(methylamino)-2,7-naphthyridin-3-yl)cyclopropanecarboxamide was synthesized using a similar procedure that was previously described in Example 289 by using N-[5-(5-bromo-1,3-benzoxazol-2-yl)-8-(methylamino)-2,7-naphthyridin-3-yl]cyclopropanecarboxamide and 2-(1,1-difluoroethyl)morpholine hydrochloride as the starting material. LCMS (ESI) m/z 509.2, [M+H]⁺. NMR (400 MHz, DMSO-d₆) δ 11.03 (s, 1H), 9.64 (s, 1H), 9.44 (s, 1H), 8.89 (s, 1H), 8.67-8.59 (m, 1H), 7.61 (d, J=8.8 Hz, 1H), 7.31 (d, J=2.4 Hz, 1H), 7.16-7.09 (m, 1H), 4.11-4.04 (m, 3.98-3.86 (m, 1H), 3.83-3.69 (m, 1H), 3.68-3.54 (m, 2H), 3.09 (d, J=4.4 Hz, 3H), 2.85-2.67 (m, 2H), 2.16-2.05 (m, 1H), 1.70 (t, J=20.0 Hz, 3H), 0.96-0.82 (m, 4H).

### Step 4: (S)—N-(5-(5-(2-(1,1-difluoroethyl)morpholino)benzo[d]oxazol-2-yl)-8-(methylamino)-2,7-naphthyridin-3-yl) cyclopropanecarboxamide and (R)—N-(5-(5-(2-(1,1-difluoroethyl)morpholino)benzo[d]oxazol-2-yl)-8-(methylamino)-2,7-naphthyridin-3-yl)cyclopropanecarboxamide

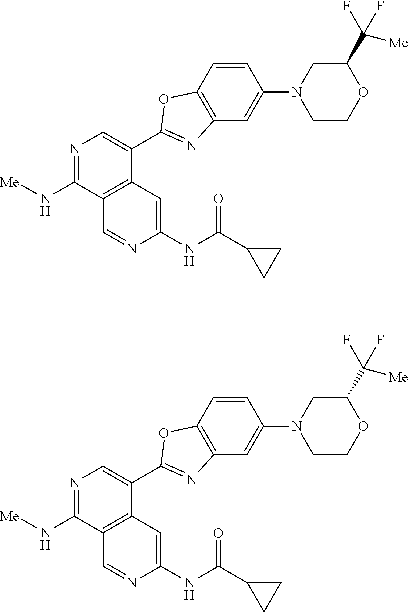 [SMILES: CNc1ncc(-c2nc3cc(N4CCO[C@H](C(C)(F)F)C4)ccc3o2)c2cc(NC(=O)C3CC3)ncc12 ; CNc1ncc(-c2nc3cc(N4CCO[C@@H](C(C)(F)F)C4)ccc3o2)c2cc(NC(=O)C3CC3)ncc12]

N-(5-(5-(2-(1,1-difluoroethyl)morpholino)benzo[d]oxazol-2-yl)-8-(methylamino)-2,7-naphthyridin-3-yl)cyclopropanecarboxamide was separated by Chiral-HPLC (Column: CHIRAL ART Cellulose-SC, 2×25 cm, 5 μm; Mobile Phase A: Hex(0.5% 2 M NH₃-MeOH), Mobile Phase B: EtOH:DCM=1:1; Flow rate: 20 mL/min; Gradient: 35% B to 35% B in 12 min; Wave Length: 220/254 nm; R_{T1}(min): 6.87; R_{T2}(min): 10.57) to afford (S)—N-(5-(5-(2-(1,1-difluoroethyl)morpholino)benzo[d]oxazol-2-yl)-8-(methylamino)-2,7-naphthyridin-3-yl)cyclopropanecarboxamide (Example 331, the faster peak) as a yellow solid and (R)—N-(5-(5-(2-(1,1-difluoroethyl)morpholino)benzo[d]oxazol-2-yl)-8-(methy amino)-2,7-naphthyridin-3-yl)cyclopropanecarboxamide (Example 332, the slower peak) as a yellow solid. The two configurations are arbitrarily assigned, LCMS (ESI) m/z 509.2, [M+H]⁺.

HNMR for example 331: ¹H NMR (400 MHz, DMSO-d₆) δ 11.03 (s, 1H), 9.63 (s, 1H), 9.44 (s, 1H), 8.89 (s, 1H), 8.67-8.61 (m, 1H), 7.61 (d, J=8.8 Hz, 1H), 7.31 (d, J=2.4 Hz, 1H), 7.12 (dd, 8.8, 2.4 Hz, 1H), 4.12-4.04 (m, 1H), 3.98-3.85 (m, 1H), 3.83-3.72 (m, 1H), 3.67-3.54 (m, 2H), 3.09 (d, J=4.4 Hz, 3H), 2.85-2.67 (m, 2H), 2.16-2.05 (m, 1H), 1.70 (t, J=20.0 Hz, 3H), 0.96-0.81 (m, 4H).

HNMR for example 332: ¹H NMR (400 MHz, DMSO-d₆) δ 11.03 (s, 1H), 9.63 (s, 1H), 9.44 (s, 1H), 8.89 (s, 1H), 8.67-8.61 (m, 1H), 7.61 (d, J=8.8 Hz, 1H), 7.31 (d, J=2.4 Hz, 1H), 7.12 (dd, J=8.8, 2.4 Hz, 1H), 4.12-4.04 (m, 1H), 3.98-3.85 (m, 1H), 3.83-3.72 (m, 1H), 3.67-3.54 (m, 2H), 3.09 (d, J=4.4 Hz, 3H), 2.85-2.67 (m, 2H), 2.16-2.05 (m, 1H), 1.70 (t, J=20.0 Hz, 3H), 0.96-0.81 (m, 4H).

### Example 333: Synthesis of N-(5-(5-(2-(hydroxymethyl)morpholino)benzo[d]oxazol-2-yl)-8-(methylamino)-2,7-naphthyridin-3-yl)cyclopropanecarboxamide

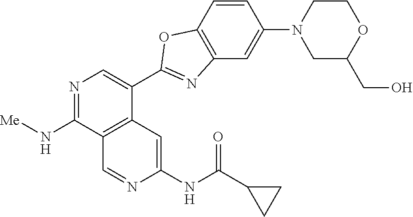 [SMILES: CNc1ncc(-c2nc3cc(N4CCOC(CO)C4)ccc3o2)c2cc(NC(=O)C3CC3)ncc12]

### Step 1: N-(5-(5-(2-((benzyloxy)methyl)morpholino)benzo[d]oxazol-2-yl)-8-(methylamino)-2, 7-naphthyridin-3-yl)cyclopropanecarboxamide

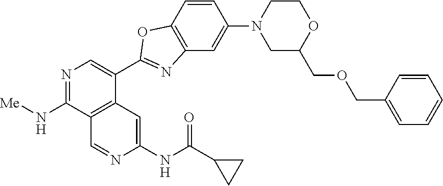 [SMILES: CNc1ncc(-c2nc3cc(N4CCOC(COCc5ccccc5)C4)ccc3o2)c2cc(NC(=O)C3CC3)ncc12]

A solution of 2-((benzyloxy)methyl)morpholine (166.8 mg; 0.684 mmol; 3.00 eq.), N-(5-(5-bromobenzo[d]oxazol-2-yl)-8-(methylamino)-2,7-naphthyridin-3-yl)cyclopropanecarboxamide (100.0 mg; 0.228 mmol; 1.00 eq.), EPhos Pd G₄ (41.9 mg; 0.046 mmol; 0.20 eq.), EPhos (24.4 mg; 0.046 mmol; 0.20 eq.) and Cs₂CO₃ (297.3 mg; 0.912 mmol; 4.00 eq.) in 1,4-dioxane (2 mL) was stirred at 100° C. overnight under nitrogen atmosphere. The desired product was detected via LCMS. The resulting mixture was concentrated under reduced pressure. The residue was purified by flash chromatography on silica gel column using 5-10% of MeOH in CH₂Cl₂ as eluent to afford N-(5-(5-(2-((benzyloxy)methyl)morpholino)benzo[d]oxazol-2-yl)-8-(methylamino)-2,7-naphthyridin-3-yl)cyclopropanecarboxamide as a yellow solid (80.0 mg, 57.8%). LCMS (ESI) m/z 565.2, [M+H]⁺.

### Step 2: N-(5-(5-(2-(hydroxymethyl)morpholino)benzo[d]oxazol-2-yl)-8-(methylamino)-2,7-naphthyridin-3-yl)cyclopropanecarboxamide

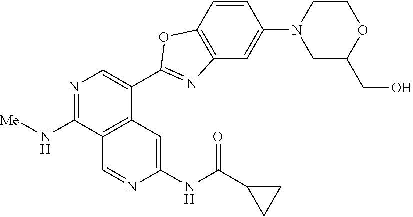 [SMILES: CNc1ncc(-c2nc3cc(N4CCOC(CO)C4)ccc3o2)c2cc(NC(=O)C3CC3)ncc12]

To a solution of N-(5-(5-(2-((benzyloxy)methyl)morpholino)benzo[d]oxazol-2-yl)-8-(methylamino)-2,7-naphthyridin-3-yl)cyclopropanecarboxamide (80.0 mg; 0.142 mmol; 1.00 eq.) in MeOH (15 mL) was added 10% Pd/C (80.0 mg; 100% w/w) under nitrogen atmosphere. The mixture was hydrogenated at room temperature overnight under hydrogen atmosphere (2 atm). The desired product was detected via LCMS. After filtration, the filtrate was concentrated under reduced pressure. The residue was purified by flash chromatography on pre-packed CIS column using 50-80% of MeOH in water (10 mmol/L NH₄HCO₃) as eluent to provide N-(5-(5-(2-(hydroxy methyl)morpholino)benzo[d]oxazol-2-yl)-8-(methylamino)-2,7-naphthyridin-3-yl)cyclopropanecarboxamide as a yellow solid (7.7 mg, 11.3%). LCMS (ESI) m/z 475.2, [M+H]⁺. ¹H NMR (400 MHz, DMSO-d₆) δ 11.03 (s, 1H), 9.66 (s, 1H), 9.44 (s, 1H), 8.89 (s, 1H), 8.65-8.59 (m, 1H), 7.59 (d, J=8.8 Hz, 1H), 7.22 (d, 2.4 Hz, 1H), 7.11-7.02 (m, 1H), 4.85-4.78 (m, 1H), 4.01-3.93 (m, 1H), 3.75-3.68 (m, 1H), 3.68-3.57 (m, 2H), 3.57-3.43 (m, 3H), 3.09 (d, J=4.4 Hz, 3H), 2.79-2.70 (m, 1H), 2.50-2.46 (m, 1H), 2.14-2.06 (m, 1H), 0.98-0.88 (m, 4H).

### Example 334: Synthesis of N-(8-(methylamino)-5-(5-(6-m ethylene-1,4-oxazepan-41-yl)benzo[d]oxazol-2-yl)-2,7-naphthyridin-3-yl)cyclopropanecarboxamide

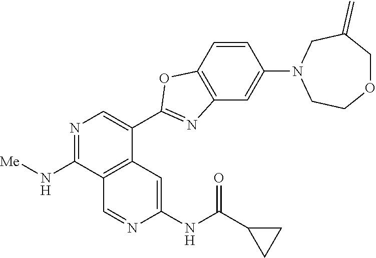 [SMILES: C=C1COCCN(c2ccc3oc(-c4cnc(NC)c5cnc(NC(=O)C6CC6)cc45)nc3c2)C1]

A solution of 4-oxa-7-azaspiro[2.5]octane hydrochloride (81.9 mg; 0.549 mmol; 3.00 eq.) and N-(5-(5-bromobenzo[d]oxazol-2-yl)-8-(methylamino)-2,7-naphthyridin-3-yl)cyclopropanecarboxamide (80.0 mg; 0.183 mmol; 1.00 eq.) in dioxane (4 mL). To the above solution was added EPhos Pd G₄ (19.5 mg; 0.037 mmol; 0.20 eq.), EPhos (19.5 mg; 0.037 mmol; 0.20 eq.) and Cs₂CO₃ (237.8 mg; 0.732 mmol; 4.00 eq.) under nitrogen atmosphere. The resulting mixture was stirred at 100° C. overnight. The resulting mixture was concentrated under reduced pressure. The residue was purified by flash chromatography on silica gel column using 0-10% of MeOH in CH₂Cl₂ as eluent to provide N-(8-(methylamino)-5-(5-(6-methylene-1,4-oxazepan-4-yl)benzo[d]oxazol-2-yl)-2,7-naphthyridin-3-yl)cyclopropanecarboxamide as a yellow solid (22.0 mg, 24.4%). LCMS (ESI) m/z 471.1, [MH-H]⁺. ¹H NMR (400 MHz, DMSO-d₆) 11.01 (s, 1H), 9.54 (s, 1H), 9.43 (s, 1H), 8.85 (s, 1H), 8.69-8.39 (m, 1H), 7.52 (d, J=8.8 Hz, 1H), 7.03 (d, J=2.4 Hz, 1H), 6.90-6.71 (m, 1H, 5.18 (s, 1H), 5.08 (s, 1H), 4.29 (s, 2H), 3.96 (s, 2H), 3.87-3.70 (m, 4H), 3.08 (d, J=4.4 Hz, 3H), 2.17-1.98 (m, 1H), 0.97-0.79 (m, 4H).

### Example 335: Synthesis of N-(5-(5-(6-methyl-1,4-oxazepan-4-yl)benzo[d]oxazol-2-yl)-8-(methylamino)-2,7-naphthyridin-3-yl)cyclopropanecarboxamide

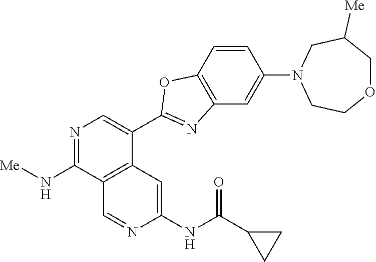 [SMILES: CNc1ncc(-c2nc3cc(N4CCOCC(C)C4)ccc3o2)c2cc(NC(=O)C3CC3)ncc12]

To a solution of N-(8-(methylamino)-5-(5-(6-methylene-1,4-oxazepan-4-yl)benzo[d]oxazol-2-yl)-2,7-naphthyridin-3-O cyclopropanecarboxamide (Example 334) (16.0 mg; 0.034 mmol; 1.00 eq.) in MeOH (10 mL) was added 10% Pd/C (5.3 mg; 30% w/w) under nitrogen atmosphere. The mixture was hydrogenated at 40° C. for 2 hours under hydrogen atmosphere (2 atm). After filtering through a celite pad, the filtrate was concentrated under reduced pressure. The residue was purified by flash chromatography on pre-packed C18 column using 20-50% of MeCN in water (10 NH₄HCO₃) as eluent to provide N-(5-(5-(6-methyl-1,4-oxazepan-4-yl)benzo[d]oxazol-2-yl)-8-(methylamino)-2,7-naphthyridin-3-yl)cyclopropanecarboxamide as a yellow solid (5.2 mg, 32.3%). LCMS (ESI) m/z 473.2, [M+H]⁺. ¹H NMR (400 MHz, DMSO-d₆) δ 11.01 (s, 1H), 9.57 (s, 1H), 9.43 (s, 1H), 8.86 (s, 1H), 8.64-8.55 (m, 1H), 7.50 (d, J=9.0 Hz, 1H), 7.00 (d, J=2.5 10 Hz, 1H), 6.84 (dd, J=9.0, 2.5 Hz, 1H), 3.94-3.79 (m, 3H), 3.78-3.66 (m, 1H), 3.57-3.49 (m, 1H), 3.48-3.42 (m, 1H), 3.42-3.34 (m, 1H), 3.12-3.00 (m, 4H), 2.30-2.17 (m, 1H), 2.14-2.05 (m, 1H), 0.94-0.88 (m, SH), 0.89-0.83 (m, 2H).

### Example 336: Synthesis of N-(8-(methylamino)-5-(5-(oxetan-3-ylmethoxy)benzo[d]oxazol-2-yl)-2,7-naphthyridin-3-yl)cyclopropanecarboxamide

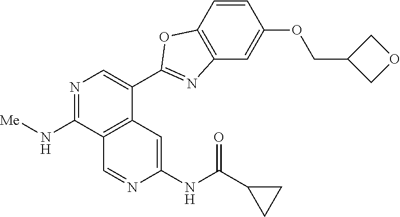 [SMILES: CNc1ncc(-c2nc3cc(OCC4COC4)ccc3o2)c2cc(NC(=O)C3CC3)ncc12]

### Step 1: N-(5-(5-hydroxybenzo[d]oxazol-2-yl)-8-methylamino)-2,7-naphthyridin-3-yl)cyclopropanecarboxamide

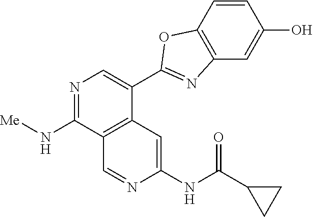 [SMILES: CNc1ncc(-c2nc3cc(O)ccc3o2)c2cc(NC(=O)C3CC3)ncc12]

To a solution of N-(5-(5-(benzyloxy)(benzo[d]oxazol-2-yl)-8-(methylamino)-2,7-naphthyridin-3-yl)cyclopropanecarboxamide (Example 234) (1.36 g; 2.922 mmol; 1.00 eq.) in a mixture solvent of DMA/MeOH (1:1, 40 mL) was added 10% Pd/C (3.40 g, 250% w/w) under nitrogen atmosphere. The mixture was hydrogenated at room temperature for 1 hour under hydrogen atmosphere (2 atm). The desired product was detected via. LCMS. The mixture was filtered through a celite pad and the filtrate was concentrated under reduced pressure. The residue was purified by flash chromatography on pre-packed C18 column using 70-90% of MeOH in water (10 mmol/L NH₄HCO₃) as eluent to provide N-(5-(5-hydroxybenzo[d]oxazol-2-yl)-8-(methylamino)-2,7-naphthyridin-3-yl)cyclopropanecarboxamide as a brown solid (800.0 mg, 72.1%). LCMS (ESI) m/z 376.1, [M+H]⁺.

### Step 2: N-(8-(methylamino)-5-(5-(oxetan-3-ylmethoxy)benzo[d]oxazol-2-yl)-2,7-naphthyridin-3-yl)cyclopropanecarboxamide

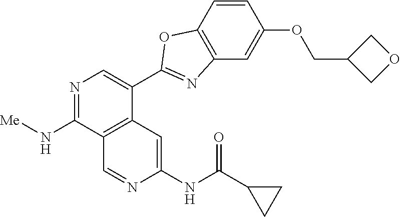 [SMILES: CNc1ncc(-c2nc3cc(OCC4COC4)ccc3o2)c2cc(NC(=O)C3CC3)ncc12]

A solution of N-(5-(5-hydroxybenzo[d]oxazol-2-yl)-8-(methylamino)-2,7-naphthyridin-3-yl)cyclopropanecarboxamide (50.0 mg; 0.133 mmol; 1.00 eq.), 3-(bromomethyl)oxetane (20.1 mg; 0.133 mmol; 1.00 eq.) and Cs₂CO₃ (86.8 mg; 0.266 mmol; 2.00 eq.) in DMF (2 mL) was stirred at 50° C. for 1 hour. The desired product was detected via LCMS. The reaction solution was diluted with EtOAc (50 mL), washed with brine (3×10 mL), dried over Na₂SO₄, filtered, and concentrated under reduced pressure. The residue was purified by flash chromatography on silica gel column using 0-20% of MeOH in CH₂Cl₂ as eluent to provide the crude product. The crude product was purified by flash chromatography on pre-packed C18 column using 60-80% of MeCN in water (10 mmol/L NH₄HCO₃) as eluent to provide N-(8-(methylamino)-5-(5-(oxetan-3-ylmethoxy)benzo[d]oxazol-2-yl)-2,7-naphthyridin-3-yl)cyclopropanecarboxamide as a yellow solid (14.7 mg, 241%). LCMS (ISI) m/z 446.2, [M+H]⁺. ¹H NMR (400 MHz, DMSO-d₆) δ 11.02 (s, 1H), 9.65 (s, 1H), 9.43 (s, 1H), 8.89 (s, 1H), 8.66-8.61 (m, 1H), 7.61 (d, J=8.8 Hz, 1H), 7.33 (d, J=2.4 Hz, 1H), 6.98 (dd, J=8.8, 2.4 Hz, 1H), 4.78-4.70 (m, 2H), 4.51-4.44 (m, 2H), 4.31 (d, J=6.8 Hz, 2H), 3.50-3.40 (m, 1H), 3.09 (d, J=4.4 Hz, 3H), 2.15-2.05 (m, 1H), 0.97-0.87 (m, 4H).

### Examples 337 and 338

Each compound in Table 14 below was prepared using a similar experimental procedure to prepare Example 336, using N-(5-(5-hydroxybenzo[d]oxazol-2-yl)-8-(methylamino)-2,7-naphthyridin-3-yl)cyclopropanecarboxamide as the common intermediate and appropriate alkyl halide,

TABLE 14

| Example | Structure and Name | MS and ¹H NMR |
|---|---|---|
| Example 337 | 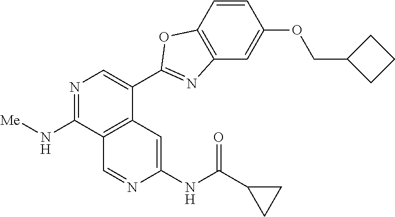 [SMILES: CNc1ncc(-c2nc3cc(OCC4CCC4)ccc3o2)c2cc(NC(=O)C3CC3)ncc12] | LCMS (ESI) m/z 444.2, [M + H]⁺. ¹H NMR (400 MHz, DMSO-d₆) δ 11.03 (s, 1H), 9.65 (s, 1H), 9.44 (s, 1H), 8.89 (s, 1H), 8.66-8.61 (m, 1H), 7.60 (d, J = 8.8 Hz, 1H), 7.27 (d, J = 2.4 Hz, 1H), 6.95 (dd, J = 8.8, 2.4 Hz, 1H), 4.04 (d, J = 6.8 Hz, 2H), 3.09 (d, J = 4.4 Hz, 3H), 2.81-2.72 (m, 1H), 2.15-2.05 (m, 3H), 1.99-1.81 (m, 4H), 0.97-0.81 (m, 4H). |
| | N-(5-(5- | |
| | (cyclobutylmethoxy)benzo[d]oxazol- | |
| | 2-yl)-8-(methylamino)-2,7- | |
| | naphthyridin-3- | |
| | yl)cyclopropanecarboxamide | |
| Example 338 | 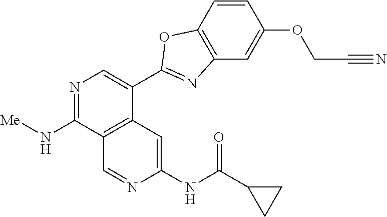 [SMILES: CNc1ncc(-c2nc3cc(OCC#N)ccc3o2)c2cc(NC(=O)C3CC3)ncc12] | LCMS (ESI) m/z 415.1, [M + H]⁺, ¹H NMR (400 MHz, DMSO-d₆) δ 11.05 (s, 1H), 9.66 (s, 1H), 9.44 (s, 1H), 8.92 (s, 1H), 8.671-8.63 (m, 1H), 7.71 (d, J = 8.8 Hz, 1H), 7.50 (d, J = 2.4 Hz, 1H), 7.09 (dd, J = 8.8, 2.4 Hz, 1H), 5.39 (s, 2H), 3.09 (d, J = 4.4 Hz, 3H), 2.13-2.08 (m, 1H), 0.95-0.83 (m, 4H). |
| | N-(5-(5- | |
| | (cyanomethoxy)benzo[d]oxazol-2- | |
| | yl)-8-(methylamino)-2,7- | |
| | naphthyridin-3- | |
| | yl)cyclopropanecarboxamide | |

### Example 339: Synthesis of N-(5-(5-(fluoromethoxy)benzo[d]oxazol-2-yl)-8-(methylamino 2,7-naphthyridin-3-yl)cyclopropanecarboxamide

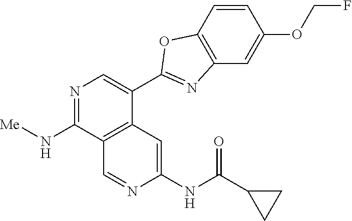 [SMILES: CNc1ncc(-c2nc3cc(OCF)ccc3o2)c2cc(NC(=O)C3CC3)ncc12]

To a stirred mixture of N-(5-(5-hydroxybenzo[d]oxazol-2-yl)-8-(methylamino)-2,7-naphthyridin-3-yl)cyclopropanecarboxamide (90.0 mg; 0.240 mmol; 1.00 eq.) and Cs₂CO₃ (234.3 mg; 0.719 mmol; 3.00 eq.) in DMF (5 mL) was added fluoroiodomethane (57.5 mg; 0.360 mmol; 1.50 eq.). The resulting mixture was stirred at 60° C. for 2 hours. The mixture was purified by flash chromatography on pre-packed C18 column using 20-60% of MeOH in water (10 mmol/L NH₄HCO₃) to provide N-(5-(5-(fluoromethoxy)benzo[d]oxazol-2-yl)-8-(methylamino)-2,7-naphthyridin-3-yl)cyclopropanecarboxamide as a yellow solid (53.8 mg, 52.1%), LCMS (ESI) m/z 408.1, [M+H]⁺. ¹H NMR (400 MHz, DMSO-d₆) δ 11.06 (5, 1H), 9.73 (s, 1H), 9.43 (s, 1H), 8.91 (s, 1H), 8.79-8.62 (m, 1H), 7.71 (d, J=8.8 Hz, 1H), 7.48 (d, J=2.4 Hz, 1H), 7.19-7.07 (m, 1H), 6.00 (d, J=54.4 Hz, 2H), 3.08 (d, J=4.4 Hz, 3H), 2.16-2.07 (m, 1H), 0.96-0.82 (m, 4H).

### Example 340: Synthesis of N-(8-(methylamino)-5-(5-(pyridin-4-yloxy)benzo[d]oxazol-2-yl)-2,7-naphthyridin-3-yl)cyclopropanecarboxamide

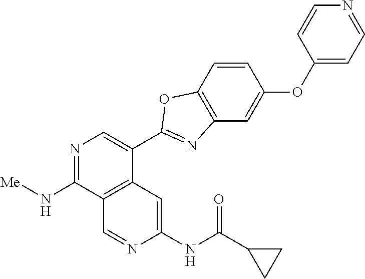 [SMILES: CNc1ncc(-c2nc3cc(Oc4ccncc4)ccc3o2)c2cc(NC(=O)C3CC3)ncc12]

A solution of N-(5-(5-hydroxybenzo[d]oxazol-2-yl)-8-(methylamino)-2,7-naphthyridin-3-yl)cyclopropanecarboxamide (45.0 mg; 0.120 mmol; 1.00 eq.), 4-iodopyridine (49.2 mg; 0.240 mmol; 2.00 eq.), (1R,2R)—N¹,N²-dimethylcyclohexane-1,2-diamine (6.8 mg; 0.048 mmol; 0.40 eq.), Cut (4.6 mg; 0.024 mmol; 0.20 eq.) and K₂CO₃ (33.1 mg; 0.239 mmol; 2.00 eq.) in DMSO (3 mL) was stirred at 120° C. overnight under nitrogen atmosphere. The desired product was detected via LCMS. The mixture was purified by flash chromatography on pre-packed C18 column using 20-80% of MeOH in water (10 mmol/L NH₄HCO₃) as eluent to provide N-(8-(methylamino)-5-(5-(pyridin-4-yloxy)benzo[c]oxazol-2-yl)-2,7-naphthyridin-3-yl)cyclopropanecarboxamide as a yellow solid (25.8 mg, 47.1%). LCMS (ESI) m/z 453.2, [M+H]⁺. ¹H NMR (400 MHz, DMSO-d₆) δ 11.03 (s, 1H), 9.64 (s, 1H), 9.45 (s, 1H), 8.94 (s, 1H), 8.72-8.09 (m, 1H), 8.49-8.43 (m, 2H), 7.83 (d, J=8.8 Hz, 1H), 7.57 (d, J===2.4 Hz, 1H), 7.21 (dd, J=8.8, 2.4 Hz, 1H), 6.99-6.93 (m, 2H), 3.10 (d, J=4.4 Hz, 3H), 2.12-2.05 (m, 1H), 0.91-0.82 (m, 4H).

### Example 341: Synthesis of N-(5-(5-((1-methyl-1H-pyrazol-3-yl)oxy)benzo[d]oxazol-2-yl)-8-(methylamino)-2,7-naphthyridin-3-yl)cyclopropanecarboxamide

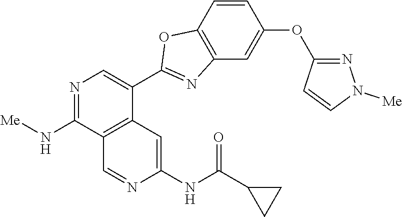 [SMILES: CNc1ncc(-c2nc3cc(Oc4ccn(C)n4)ccc3o2)c2cc(NC(=O)C3CC3)ncc12]

A solution of N-(5-(5-hydroxybenzo[d]oxazol-2-yl)-8-(methylamino)-2,7-naphthyridin-3-yl)cyclopropanecarboxamide (50.0 mg; 0.133 mmol; 1.00 eq.), 3-iodo-1-methyl-1H-pyrazole (83.1 mg; 0.399 mmol; 3.00 eq.), (1R,2R)—N¹,N²-dimethylcyclohexane-1,2-diamine (28.4 mg, 0.200 mmol; 1.50 eq.), CuI (19.0 mg; 0.100 mmol; 0.75 eq.) and K₂CO₃ (36.8 mg; 0.266 mmol; 2.00 eq.) in DMSO (9 mL) was stirred at 200° C. for 2 hours under microwave conditions. The desired product was detected via LCMS. The resulting mixture was concentrated under vacuum and purified by flash chromatography on silica gel column using 0-10% of MeOH in CH₂Cl₂ as eluent to afford the crude product. The crude product was purified by re-crystallization with a mixture solvent of MeOH/CH₂Cl₂ (1:10, 5 mL) to provide N-(5-(5-((1-methyl-1H-pyrazol-3-yl)oxy)benzo[d]oxazol-2-yl)-8-(methylamino)-2,7-naphthyridin-3-yl)cyclopropanecarboxamide as a yellow solid (7.6 mg, 11.7%). LCMS (ESI) m/z 456.2, [M+H]⁺. ¹H NMR (400 MHz, DMSO-d₆) δ 11.03 (s, 1H), 9.64 (s, 1H), 9.44 (s, 1H), 8.91 (s, 1H), 8.69-8.64 (m, 1H), 7.70 (d, J=8.8 Hz, 1H), 7.66 (d, J===2.0 Hz, 1H), 7.37 (d, J=2.4 Hz, 1H), 7.13 (dd, J=8.8, 2.4 Hz, 1H), 5.86 (d, J=2.0 Hz, 1H), 3.77 (s, 3H), 3.09 (d, J=4.4 Hz, 3H), 2.12-2.06 (m, 1H), 0.95-0.82 (m, 4H).

### Example 342: Synthesis of N-(5-(5-(3,6-dihydro-2H-pyran-4-yl)benzo[d]oxazol-2-yl)-8-(methylamino)-2,7-naphthyridin-3-yl)cyclopropanecarboxamide

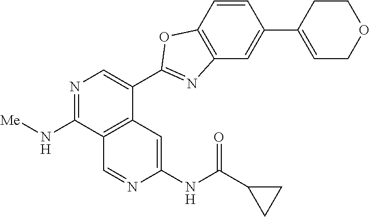 [SMILES: CNc1ncc(-c2nc3cc(C4=CCOCC4)ccc3o2)c2cc(NC(=O)C3CC3)ncc12]

To a solution of N-(5-(5-bromobenzo[d]oxazol-2-yl)-8-(methylamino)-2,7-naphthyridin-3-yl)cyclopropanecarboxamide (40.0 mg; 0.091 mmol; 1.00 eq.) in a mixture solvent of 1,4-dioxane/water (5:1, 3.6 mL) were added 2-(3,6-dihydro-2H-pyran-4-yl)-4,4,5,5-tetramethyl-1,3,2-dioxaborolane (19.1 mg; 0.091 mmol; 1.00 eq.), XPhos (8.7 mg; 0.018 mmol; 0.20 eq.), XPhos Pd G₃ (15.4 mg; 0.018 mmol; 0.20 eq.), and K₃PO₄ (58.1 mg; 0.273 mmol; 3.00 eq.) under nitrogen atmosphere. The reaction was stirred at 90° C. for 1 hour. The desired product was detected via LCMS. The resulting mixture was concentrated under reduced pressure. The residue was purified by flash chromatography on silica gel column using 2-10% of MeOH in CH₂Cl₂ as eluent to provide N-(5-(5-(3,6-dihydro-2H-pyran-4-yl)benzo[d]oxazol-2-yl)-8-(methylamino)-2,7-naphthyridin-3-yl)cyclopropanecarboxamide (crude). The crude product was purified by flash chromatography on pre-packed C18 column using 20-80% of MeOH in water (10 mmol/L NH₃HCO₃) as eluent to provide N-(5-(5-(3,6-dihydro-2H-pyran-4-yl)benzo[d]oxazol-2-yl)-8-(methylamino)-2,7-naphthyridin-3-yl)cyclopropanecarboxamide as yellow solid (5.4 mg, 13.4%). LCMS (ESI) m/z 442.2, [M+H]⁺. ¹H NMR (400 MHz, DMSO-D₆) δ 11.05 (s, 1H), 9.68 (s, 1H), 9.45 (s, 1H), 8.92 (s, 1H), 8.70-8.64 (m, 1H), 7.77 (d, J=1.6 Hz, 1H), 7.70 (d, J=8.8 Hz, 1H), 7.51 (dd, J=8.8, 1.6 Hz, 1H), 6.34 (s, 1H), 4.30-4.23 (m, 2H), 3.91-3.83 (m, 2H), 3.10 (d, 1=4.4 Hz, 3H), 2.60-2.53 (m, 2H), 2.15-2.05 (m, 1H), 0.94-0.84 (m, 4H).

### Example 343: Synthesis of N-(8-(methylamino)-5-(5-(tetrahydrofuran-2-yl)benzo[d]oxazol-2-yl)-2,7-naphthyridin-3-yl)cyclopropanecarboxamide

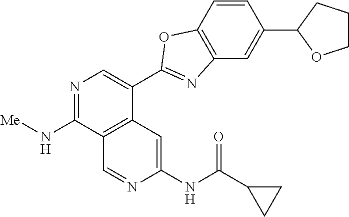 [SMILES: CNc1ncc(-c2nc3cc(C4CCCO4)ccc3o2)c2cc(NC(=O)C3CC3)ncc12]

### Step 1: N-(5-(5-(4,5-dihydrofuran-2-yl)benzo[d]oxazol-2-yl)-8-(methylamino)-2,7-naphthyridin-3-yl)cyclopropanecarboxamide

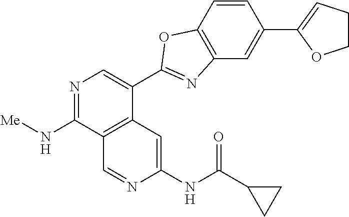 [SMILES: CNc1ncc(-c2nc3cc(C4=CCCO4)ccc3o2)c2cc(NC(=O)C3CC3)ncc12]

A solution of N-(5-(5-bromobenzo[d]oxazol-2-yl)-8-(methylamino)-2,7-naphthyridin-3-yl)cyclopropanecarboxamide (100.0 mg; 0.228 mmol; 1.00 eq.), tributyl(4,5-dihydrofuran-2-yl)stannane (245.0 mg; 0.682 mmol; 2.99 eq.), CuI (8.7 mg; 0.046 mmol; 0.20 eq.), Pd(PPh₃)₄ (52.7 mg; 0.046 mmol; 0.20 eq.) and LiCl (24.0 mg; 0.566 mmol; 2.48 eq.) in 1,4-dioxane (2.4 mL) was stirred at 100° C. for 2 hours under nitrogen atmosphere. The desired product was detected via LCMS. The mixture was concentrated under reduced pressure to provide N-(5-(5-(4,5-dihydrofuran-2-yl)benzo[d]oxazol-2-yl)-8-(methylamino)-2,7-naphthyridin-3-yl)cyclopropanecarboxamide as a black solid (414.0 mg, crude). The crude product was used for the next step without further purification. LCMS (ESI) m/z 428.2, [M+H]⁺.

### Step 2: N-(8-(methylamino)-5-(5-(tetrahydrofuran-2-yl)benzo[d]oxazol-2-yl)-2,7-naphthyridin-3-yl)cyclopropanecarboxamide

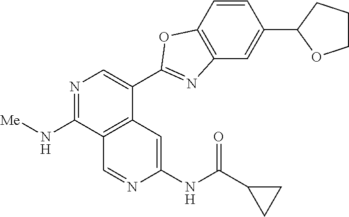 [SMILES: CNc1ncc(-c2nc3cc(C4CCCO4)ccc3o2)c2cc(NC(=O)C3CC3)ncc12]

To a solution of N-(5-(5-(4,5-dihydrofuran-2-yl)benzol oxazol-2-yl)-8-(methylamino)-2,7-naphthyridin-3-yl)cyclopropanecarboxamide (414.0 mg; 0.969 mmol; 1.00 eq.) in triethylsilane (9 mL) was added TPA (9 mL). The mixture was stirred at room temperature for 2 hours. The desired product was detected via LCMS. The reaction was quenched with water (20 mL), extracted with CH₂Cl₂ (3×30 mL). The combined organic layers were dried with Na₂SO₄ and concentrated under vacuum. The residue was purified by flash chromatography on silica gel column using 0-10% of MeOH in CH₂Cl₂ as eluent to provide the crude product. The crude product was purified by Prep-Achiral-SFC (Column: DAICEL DCpak P4VP 3×25 cm, 5 μm; Mobile Phase A: CO₂, Mobile Phase B: MeOH (0.1% 2 M NH₃-MeOH); Flow rate: 60 mL/min; Gradient: isocratic 50% B) to provide N-(8-(methylamino)-5-(5-(tetrahydrofuran-2-yl)benzo[d]oxazol-2-yl)-2,7-naphthyridin-3-yl)cyclopropanecarboxamide as a light yellow solid (17.1 mg, 4.1%). (ESI) m/z 430.2, [M+H]⁺. ¹H NMR (400 MHz, DMSO-d₆) 11.02. (s, 1H), 9.70 (s, 1H), 9.44 (s, 1H), 8.91 (s, 1H), 8.68-8.61 (m, 1H), 7.68-7.64 (m, 2H), 7.35-7.31 (m, 1H), 4.96 (t, J=7.2 Hz, 1H), 4.11-4.03 (m, 1H), 3.89-3.81 (m, 1H), 3.09 (d, J=4.4 Hz, 3H), 2.42-2.31 (m, 1H), 2.14-1.94 (m, 3H), 1.78-1.68 (m, 1H), 0.95-0.82 (m, 4H).

### Example 344: Synthesis of N-(5-(5-(2-cyanoethyl)benzo[d]oxazol-2-yl)-8-(methylamino)-2,7-naphthyridin-3-yl)cyclopropanecarboxamide

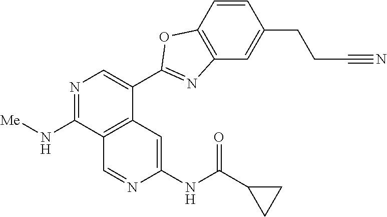 [SMILES: CNc1ncc(-c2nc3cc(CCC#N)ccc3o2)c2cc(NC(=O)C3CC3)ncc12]

### Step 1: (E)-N-(5-(5-(2-cyanovinyl)benzo[d]oxazol-2-yl)-8-(methylamino)-2,7-naphthyridin-3-yl)cyclopropanecarboxamide

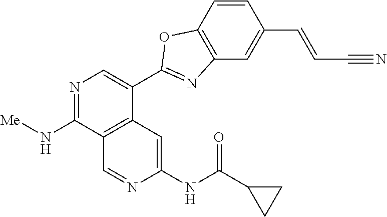 [SMILES: CNc1ncc(-c2nc3cc(/C=C/C#N)ccc3o2)c2cc(NC(=O)C3CC3)ncc12]

A solution of N-(5-(5-bromobenzo[d]oxazol-2-yl)-8-(methylamino)-2,7-naphthyridin-3-yl)cyclopropanecarboxamide (100.0 mg; 0.228 mmol; 1.00 eq.), acrylonitrile (121.0 mg; 2.280 mmol; 9.99 eq.), Pd(PPh₃)₂Cl₂ (16.0 mg; 0.023 mmol; 0.10 eq.) and Na₂CO₃ (47.9 mg; 0.452 mmol: 1.98 eq.) in DMF (2 mL) was stirred at 100° C. for 14 hours under nitrogen atmosphere. The desired product was detected via LCMS. The resulting mixture was diluted with EtOAc (50 mL) and washed with brine (3×10 mL). The organic phase was dried with Na₂SO₄ and concentrated under vacuum. The residue was purified by flash chromatography on silica gel column using 20-70% of EtOAc in petroleum ether as eluent to provide (E)-N-(5-(5-(2-cyanovinyl)benzo[d]oxazol-2-yl)-8-(methy amino)-2,7-naphthyridin-3-yl)cyclopropanecarboxamide as an orange solid (70.0 mg, 74.7%). LCMS (ESI) m/z 411.1, [M+H]⁺.

### Step 2: N-(5-(5-(2-cyanoethyl)benzo(dioxazol-2-yl)-8-(methylamino)-2,7-naphthyridin-3-yl)cyclopropanecarboxamide

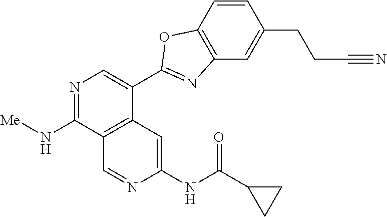 [SMILES: CNc1ncc(-c2nc3cc(CCC#N)ccc3o2)c2cc(NC(=O)C3CC3)ncc12]

To a solution of N-(5-(5-(2-cyanovinyl)benzo[d]oxazol-2-yl)-8-(methylamino)-2,7-naphthyridin-3-yl)cyclopropanecarboxamide (60.0 mg; 0.146 mmol; 1.00 eq.) in MeOH (40 mL) was added 10% Pd/C (60.0 mg; 100% w/w) under nitrogen atmosphere. The mixture was hydrogenated at 40° C. for 5 hours under hydrogen atmosphere (2 atm). The desired product was detected via LCMS. The resulting mixture was filtered, the filter cake was washed with MeOH (3×30 mL). The filtrate was collected and concentrated under reduced. The residue was purified by flash chromatography on silica gel column using 0-6% of MeOH in CH₂Cl₂ as eluent to provide the crude product. The crude product was purified by Prep-Achiral-SFC (Column: DAICEL DCpak P4VP 3×25 cm, 5 μm; Mobile Phase A: CO₂, Mobile Phase B: MeOH (0.1% 2 M NH₃-MeOH); Flow rate: 60 mL/min; Gradient: isocratic 47% B) to provide N-(5-(5-(2-cyanoethyl)benzo[d]oxazol-2-yl)-8-(methylamino)-2,7-naphthyridin-3-yl)cyclopropanecarboxamide as a yellow solid (22.8 mg, 37.5%). LCMS (ESI) m/z 413.2, [M+H]⁺. ¹H NMR (400 MHz, DMSO-d₆) δ 11.03 (s, 1H), 9.71 (s, 1H), 9.44 (s, 1H), 8.91 (s, 1H), 8.71-8.65 (m, 1H), 7.72 (d, 2.4 Hz, 1H), 7.68 (d, 8.8 Hz, 1H), 7.31 (dd, 8.8, 2.4 Hz, 1H), 3.09 (d, J=4.4 Hz, 3H), 3.04 (t, J=7.2 Hz, 2H), 2.90 (t, J=7.2 Hz, 2H), 2.14-2.06 (m, 1H), 0.94-0.82 (m, 4H).

### Example 345: Synthesis of N-8-(methylamino)-5-(5-morpholinobenzo[d]oxazol-2-yl)-2,7-naphthyridin-3-yl)cyclopropanecarboxamide

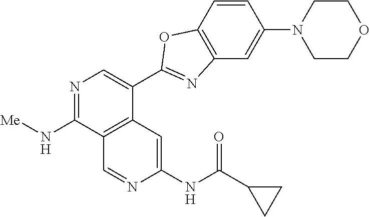 [SMILES: CNc1ncc(-c2nc3cc(N4CCOCC4)ccc3o2)c2cc(NC(=O)C3CC3)ncc12]

### Step 1: 2-methyl-5-morpholinobenzo[d]oxazole

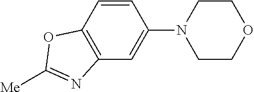 [SMILES: Cc1nc2cc(N3CCOCC3)ccc2o1]

A solution of 5-bronco-2-methylbenzo[d]oxazole (5.00 g; 23.58 mmol; 1.00 eq.), morpholine (3.29 g; 37.72 mmol; 1.60 eq.), BrettPhos Pd G₃ (3.85 g; 4.24 mmol; 0.18 eq.), BrettPhos (1.14 g; 2.12 mmol; 0.09 eq.) and Cs₂CO₃ (19.98 g; 61.32 mmol 2.60 eq.) in 1,4-dioxane (170 mL) was stirred at 100° C. overnight under nitrogen atmosphere. The desired product was detected via LCMS. The resulting mixture was concentrated under reduced pressure. The residue was purified by flash chromatography on silica gel column using 30-50% of EtOAc in petroleum ether as eluent to provide 2-methyl-5-morpholinobenzo[d]oxazole as a red oil (2.97 g, 57.1%). LCMS (ESI) m/z 219.1, [M+H]⁺.

### Step 2: 2-amino-4-morpholinophenol

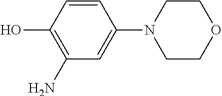 [SMILES: Nc1cc(N2CCOCC2)ccc1O]

To a solution of 2-methyl-5-morpholinobenzo[d]oxazole (2.97 g; 13,608 mmol; 1.00 eq.) in EtOH (80 mL) was added HCl (4 M in water, 24 mL). The reaction mixture was stirred at 100 DC for 4 hours. The desired product was detected via LCMS. The resulting mixture was concentrated under reduced pressure to afford 2-amino-4-morpholinophenol as yellow solid (2.97 g, crude). LCMS (ESI) m/z 195.1, [M+H]⁺.

### Step 3: 5-morpholinobenzo[d]oxazole

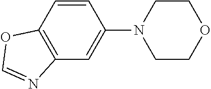 [SMILES: c1nc2cc(N3CCOCC3)ccc2o1]

A solution of 2-amino-4-morpholinophenol (2.97 g; 15,291 mmol; 1.00 eq.) in trimethyl orthoformate (80 mL) was stirred at 100° C. overnight. The desired product was detected via LCMS. The resulting mixture was concentrated under reduced pressure. The residue was purified by flash chromatography on silica gel column using 30-50% of EtOAc in petroleum ether as eluent to provide 5-morpholinobenzo[d]oxazole as a yellow solid (2.68 g, 85.1%). LCMS (ESI) m/z 205.1, [M+H]⁺. ¹H NMR (400 MHz, DMSO-d₆) δ 8.62 (s, 1H), 7.61 (d, J=8.8 Hz, 1H), 7.28 (d, 2.4 Hz, 1H), 7.13 (dd, 8.8, 2.4 Hz, 1H), 3.82-3.75 (m, 4H), 3.18-3.11 (m, 4H).

### Step 4: N-(8-(methylamino)-5-(5-morpholinobenzo[d]oxazol-2-yl)-2,7-naphthyridin-3-yl)cyclopropanecarboxamide

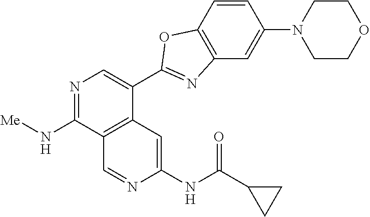 [SMILES: CNc1ncc(-c2nc3cc(N4CCOCC4)ccc3o2)c2cc(NC(=O)C3CC3)ncc12]

A stirred solution of 5-morpholinobenzo[d]oxazole (0.76 g; 3.740 mmol; 1.20 eq.), N-(5-bromo-8-(methylamino)-2,7-naphthyridin-3-yl)cyclopropanecarboxamide (1.00 g; 3.110 mmol; 1.00 eq.), Pd(PPh₃)₄ (0.36 g; 0.310 mmol; 0.10 eq.), Cs₂CO₃ (3.04 g; 9.340 mmol; 3.00 eq.) in DMF (15 mL) was stirred at 110° C. for 16 hours under nitrogen atmosphere. The solvent was removed under vacuum. The residue was purified by flash chromatography on silica gel column using 0-25% of NeOH in CH₂Cl₂ as eluent to provide N-(8-(methylamino)-5-(5-morpholinobenzo[d]oxazol-2-yl)-2,7-naphthyridin-3-yl)cyclopropanecarboxamide (1.2 g, 86.7%) as a yellow solid. LCMS (ESI) m/z 445.2, [M+H]⁺. ¹H NMR (400 MHz, DMSO-d₆) δ 11.03 (s, 1H), 9.65 (5, 1H), 9.44 (s, 1H), 8.88 (s, 1H), 8.65-8.60 (m, 1H), 7.58 (d, J=8.8 Hz, 1H), 7.23 (d, J=2.4 Hz, 1H), 7.06 (dd, 8.8, 2.4 Hz, 1H), 3.90-3.70 (m, 4H), 3.23-3.12 (m, 4H), 3.09 (d, J=4.3 Hz, 3H), 2.17-2.03 (m, 1H), 0.99-0.77 (m, 4H).

### Example 346: Synthesis of N-(5-5-(2-(2-methoxypropan-2-yl)cyclobutoxy)benzo[d]oxazol-2-yl)-8-(methylamino)-2,7-naphthyridin-3-yl)cyclopropanecarboxamide

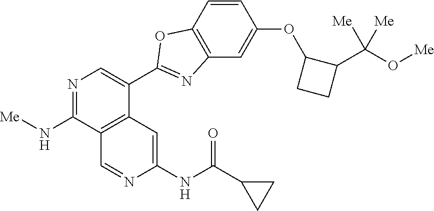 [SMILES: CNc1ncc(-c2nc3cc(OC4CCC4C(C)(C)OC)ccc3o2)c2cc(NC(=O)C3CC3)ncc12]

### Step 1: methyl 2,2-diethoxycyclobutane-1-carboxylate

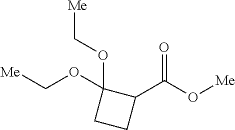 [SMILES: CCOC1(OCC)CCC1C(=O)OC]

The distilling flask was charged with t-BuOK (5.69 g; 50.743 mmol; 1.00 eq.) and 2-bromo-1,1-diethoxyethane (10.0 g; 50.743 mmol; 1.00 eq.) was slowly added at 0° C. for 3 hours under nitrogen atmosphere. The exothermic reaction turned yellow and diethoxyethene was distilled off (65° C.-170° C., oil bath) as a 1:1 mixture with t-BuOH. Methyl acrylate (2.18 g; 23.232 mmol; 0.50 eq.) was added and the reaction mixture was further diluted with t-BuOH (14 mL), stirred at 90° C. for five days. The solvent was concentrated under vacuum. The residue was purified by flash chromatography on silica gel column using 0-30% of EtOAc in petroleum ether as eluent to provide methyl 2,2-diethoxycyclobutane-1-carboxylate as a colourless liquid (3.01 g, 65.9%).

### Step 2: 2-(2,2-diethoxycyclobutyl)propan-2-ol

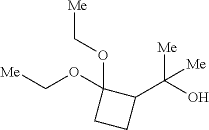 [SMILES: CCOC1(OCC)CCC1C(C)(C)O]

To a stirred solution of methyl 2,2-diethoxycyclobutane-1-carboxylate (1.50 g; 7.417 mmol; 1.00 eq.) in diethyl ether (25 mL) was added bromo(methyl)magnesium (1 M in THF) (37.1 mL; 37.1 mmol; 5.00 eq.) dropwise at 0° C. under nitrogen atmosphere. The resulting mixture was stirred at room temperature for 2 hours. The reaction was quenched with water (10 mL) at 0° C. and extracted with CH₂Cl₂(3×50 mL). The combined organic layers were dried over anhydrous Na₂SO₄, concentrated under reduced pressure to afford 2-(2,2-diethoxycyclobutyl)propan-2-ol as a colourless liquid (1.49 g, crude).

### Step 3: 1,1-diethoxy-2-(2-methoxypropan-2-yl)cyclobutane

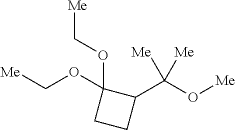 [SMILES: CCOC1(OCC)CCC1C(C)(C)OC]

To a stirred solution of 2-(2,2-diethoxycyclobutyl)propan-2-ol (750.0 mg, 3.707 mmol, 1.00 eq.) in THF (35 mL) was added NaH (60%, 296.6 mg; 7.415 mmol, 2.00 eq.) slowly at 0° C. under nitrogen atmosphere. After stirring for 30 minutes, to the above solution was added methyl iodide (1.57 g; 11.122 mmol; 3.00 eq.). The resulting solution was stirred at room temperature overnight. The reaction was quenched with ice water (10 mL) and extracted with CH₂Cl₂ (3×20 mL). The combined organic extracts were dried over anhydrous Na₂SO₄ and concentrated under vacuum. The residue was purified by flash chromatography on silica gel column using 0-30% of EtOAc in petroleum ether as eluent to provide 1,1-diethoxy-2-(2-methoxypropan-2-yl)cyclobutane as a colorless liquid (650.0 mg, 81.0%).

### Step 4: 2-(2-methoxypropan-2-yl)cyclobutan-1-one

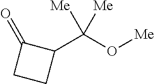

A mixture of 1,1-diethoxy-2-(2-methoxypropan-2-yl)cyclobutane (500.0 mg; 2.311 mmol; 1.00 eq.) and formic acid (1.27 g; 27.735 mmol; 12.00 eq.) was stirred at 40° C. for 5 minutes. The resulting mixture was diluted with water (10 mL) and extracted with CH₂Cl₂. (3×10 mL). The combined organic phase was concentrated under reduced pressure to afford 2-(2-methoxypropan-2-yl)cyclobutan-1-one as a colorless liquid (300.0 mg, crude). The crude product was used to next step without purification.

### Step 5: 2-(2-methoxypropan-2-yl)cyclobutan-1-ol

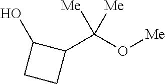

To a stirred solution of 2-(2-methoxypropan-2-yl)cyclobutan-1-one (412.0 mg; 2.897 mmol; 1.00 eq.) in THF (8 mL) was added NaBH₄ (218.1 mg; 5.765 mmol; 2.00 eq.) in portions at 0° C. under nitrogen atmosphere. The resulting mixture was stirred at room temperature for 2 hours. The reaction was quenched with saturated NH₄Cl solution at 0° C., extracted with CH₂Cl₂ (3>10 mL). The combined organic phases were concentrated under reduced pressure to afford 2-(2-methoxypropan-2-yl)cyclobutan-1-ol as a colorless oil (411.0 mg, crude).

### Step 6: 5-(2-(2-methoxypropan-2-yl)cyclobutoxy)benzo[d]oxazole

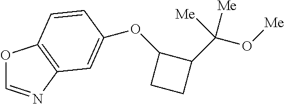 [SMILES: COC(C)(C)C1CCC1Oc1ccc2ocnc2c1]

To the mixture of 2-(2-methoxypropan-2-yl)cyclobutan-1-ol (411.0 mg; 2.850 mmol; 2.00 eq.), benzo[d]oxazol-5-ol (192.3 mg; 1.425=mmol; 1.00 eq.) and PPh₃ (746.7 mg; 2.850 mmol; 2.00 eq.) in THF (10 mL) was added DIAD (576.2 mg, 2.850 mmol, 2.00 eq.) at 0° C. The resulting solution was stirred at 50° C. overnight under nitrogen atmosphere. The desired product was detected via LCMS. The reaction was concentrated under vacuum. The residue was purified by flash chromatography on silica gel column using 0-50% of EtOAc in petroleum ether as eluent to provide 5-(2-(2-methoxypropan-2-yl)cyclobutoxy)benzo[d]oxazole as a colorless oil (24.8 mg; 3.3%).

### Step 7: N-(5-(5-(2-(2-methoxypropan-2-yl)cyclobutoxy)benzo[d]oxazol-2-yl)-8-(methylamino)-2,7-naphthyridin-3-yl)cyclopropanecarboxamide

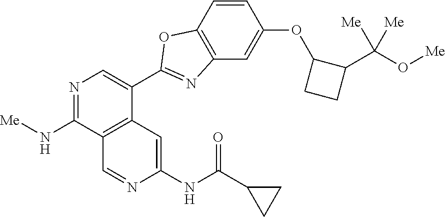 [SMILES: CNc1ncc(-c2nc3cc(OC4CCC4C(C)(C)OC)ccc3o2)c2cc(NC(=O)C3CC3)ncc12]

N-(5-(5-(2-(2-methoxypropan-2-yl)cyclobutoxy)benzo[d]oxazol-2-yl)-8-(methylamino)-2,7-naphthyridin-3-yl)cyclopropanecarboxamide was synthesized using a similar procedure that was previously described in Example 345 by using N-(5-bromo-8-(methylamino)-2,7-naphthyridin-3-yl)cyclopropanecarboxamide and 5-(2-(2-methoxypropan-2-yl)cyclobutoxy)benzo[d]oxazole as the starting material. LCMS (ESI) m/z 502.2, [M+H]⁺. ¹H NMR (400 MHz, DMSO-d₆) δ 11.04 (s, 1H), 9.63 (s, 1H), 9.44 (s, 1H), 8.89 (s, 1H), 8.70-8.62 (m, 1H), 7.65-7.55 (m, 1H), 7.23-7.13 (m, 1H), 6.97-6.87 (m, 1H), 4.70-4.60 (m, 1H), 4.15-4.33 (m, 1H), 3.16 (s, 3H), 3.09 (d, J=4.4 Hz, 3H), 2.64-2.56 (m, 1H), 2.43-2.32 (m, 1H), 2.15-2.05 (m, 1H), 1.80-1.68 (m, 1H), 1.64-1.53 (m, 1H), 1.26 (d, J=4.4 Hz, 3H), 1.11 (d, J=11.4 Hz, 3H), 0.95-0.81 (m, 4H).

### Example 347: Synthesis of N-(5-(5-((1s,3s)-3-(methoxymethyl)cyclobutoxy)benzo[d]oxazol-2-yl)-8-(methylamino)-2,7-naphthyridin-3-yl)cyclopropanecarboxamide

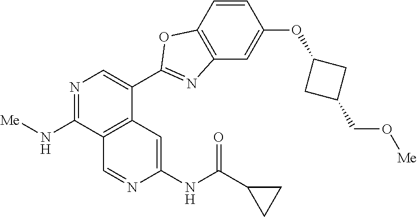 [SMILES: CNc1ncc(-c2nc3cc(O[C@H]4C[C@@H](COC)C4)ccc3o2)c2cc(NC(=O)C3CC3)ncc12]

### Step 1: methyl (1s,3s)-3-(benzo[d]oxazol-5-yloxy)cyclobutane-1-carboxylate

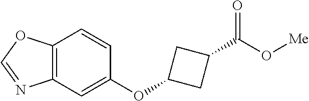 [SMILES: COC(=O)[C@H]1C[C@@H](Oc2ccc3ocnc3c2)C1]

To a solution of benzo[d]oxazol-5-ol (200.0 mg; 1.480 mmol; 1.00 eq.), methyl (1r,3r)-3-hydroxycyclobutane-1-carboxylate (154.0 mg; 1.183 mmol; 0.80 eq.) and PPh₃ (216.0 mg; 0.824 mmol; 1.11 eq.) in THF (17 mL) was added DIAD (150.0 mg; 0.742 mmol; 1.00 eq.) dropwise. The resulting mixture was stirred at room temperature for 14 hours. The desired product was detected via LCMS. The reaction was concentrated under reduced pressure. The residue was purified by flash chromatography on silica gel column using 45-55% of EtOAc in petroleum ether as eluent to provide methyl (1s,3s)-3-(benzo[d]oxazol-5-yloxy)cyclobutane-1-carboxylate as a pink solid (304.4 mg, 83.2%). LCMS (ESI) m/z 248.1, [M+H]⁺.

### Step 2: t(1s,3s)-3-(benzo[d]oxazol-5-yloxy)cyclobutyl)methanol

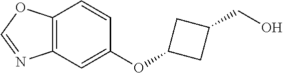 [SMILES: OC[C@H]1C[C@@H](Oc2ccc3ocnc3c2)C1]

To a stirred solution of methyl (1s,2s)-3-(benzo[d]oxazol-5-yloxy)cyclobutane-1-carboxylate (304.0 mg; 1.230 mmol; 1.00 eq.) in MeOH (5 mL) was added NaBH₄ (468.1 mg; 12.371 mmol; 10.06 eq.) in portions at 0° C. under nitrogen atmosphere. The resulting mixture was stirred at room temperature for 5 hours. The desired product was detected via LCMS. The reaction was quenched with saturated-NH₄Cl solution (20 mL), and extracted with EtOAc (2×30 mL). The organic layer was dried over anhydrous Na₂SO₄. After filtration, the filtrate was concentrated under reduced pressure. The residue was purified by flash chromatography on silica gel column using 10-25% of EtOAc in petroleum ether as eluent to provide (1s,3s)-3-(benzo[d]oxazol-5-yloxy)cyclobutylmethanol as a brown solid (80.0 mg, 29.1%). LCMS (ESI) m/z 220.1, [M+H]⁺.

### Step 3: 5-((1s,3s)-3-(methoxymethyl)cyclobutoxy)benzo[d]oxazole

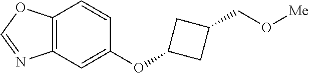 [SMILES: COC[C@H]1C[C@@H](Oc2ccc3ocnc3c2)C1]

To a stirred solution of ((1s,3s)-3-(benzo[d]oxazol-5-yloxy)cyclobutyl)methanol (80.0 mg; 0.365 mmol; 1.00 eq.) in THF (8 mL) was added NaH (60%) (74.0 mg; 3.084 mmol; 8.45 eq.) at 0° C. under nitrogen atmosphere. After stirring at room temperature for 0.5 hour, to the above mixture was added iodomethane (104.0 mg; 0.733 mmol; 2.01 eq.) dropwise at 0° C. The resulting mixture was stirred at room temperature for 2 hours. The desired product was detected via LCMS. The reaction was quenched with saturated NH₄Cl solution (20 mL). The resulting mixture was extracted with EtOAc, (3×20 mL). The combined organic layers were washed with brine (2×5 mL), dried over anhydrous Na₂SO₄. After filtration, the filtrate was concentrated under reduced pressure. The residue was purified by flash chromatography on silica gel column using 30-55% of EtOAc in petroleum ether as eluent to provide 5-((1s,3s)-3-(methoxymethyl)cyclobutoxy)benzo[d]oxazole as a colorless oil (66.0 mg, 77.1%), LCMS (ESI) m/z 234.1, [M+H]⁺.

### Step 4: N-(5-(5-((1s,3s)-3-(methoxymethyl)cyclobutoxy)benzo[d]oxazol-2-yl)-8-(methylamino-2,7-naphthyridin-3-yl)cyclopropanecarboxamide

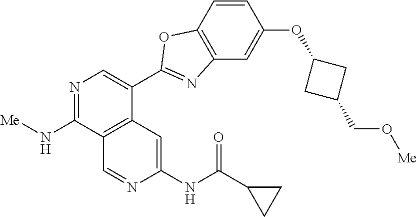 [SMILES: CNc1ncc(-c2nc3cc(O[C@H]4C[C@@H](COC)C4)ccc3o2)c2cc(NC(=O)C3CC3)ncc12]

N-(5-(5-((1s,3s)-3-(methoxymethyl)cyclobutoxy)benzo[d]oxazol-2-yl)-8-(methylamino)-2, naphthyridin-3-yl)cyclopropanecarboxamide was synthesized using a similar procedure that was previously described in Example 345 by using N-(5-bromo-8-(methylamino)-2,7-naphthyridin-3-yl)cyclopropanecarboxamide and 5-((1s,3s)-3-(methoxymethyl)cyclobutoxy)benzo[d]oxazole as the starting material. LCMS (ESI) m/z 474.2, [M+H]⁺. ¹H NMR (400 MHz, DMSO-d₆) δ 11.03 (s, 1H), 9.59 (s, 1H), 9.43 (s, 1H), 8.87 (5, 1H), 8.66-8.61 (m, 1H), 7.60 (d, J=8.8 Hz, 1H), 7.13 (d, J=2.4 Hz, 11H), 6.89 (dd, J=8.8, 2.4 HZ, 1H), 4.75-4.65 (m, 1H), 3.36 (d, J=6.0 Hz, 2H), 3.26 (s, 3H), 3.08 (d, J=4.4 Hz, 3H), 2.60-2.53 (m, 2H), 2.30-2.08 (m, 2H), 1.90-1.80 (m, 2H), 0.95-0.81 (m, 4H).

### Example 348: Synthesis of N-(5-(5-((1r,3r)-3-(methoxymethyl)cyclobutoxy)benzo[d]oxazol-2-yl)-8-(methylamino)-2,7-naphthyridin-3-yl)cyclopropanecarboxamide

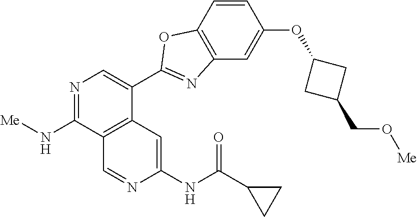 [SMILES: CNc1ncc(-c2nc3cc(O[C@H]4C[C@H](COC)C4)ccc3o2)c2cc(NC(=O)C3CC3)ncc12]

### Step 1: methyl (1r,3r)-3-(benzo[d]oxazol-5-yloxy)cyclobutane-1-carboxylate

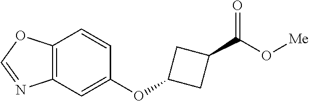 [SMILES: COC(=O)[C@H]1C[C@H](Oc2ccc3ocnc3c2)C1]

To a solution of benzo[d]oxazol-5-ol (100.0 mg; 0.740 mmol, 1.00 eq.), methyl (1s,3s)-3-hydroxycyclobutane-1-carboxylate (77.0 mg; 0.592 mmol; 0.80 eq.) and PPh₃ (216.1 mg; 0.824 mmol; 1.11 eq.) in THF (5 mL) was added DIAD (149.6 mg; 0.740 mmol; 1.00 eq.) dropwise at room temperature under nitrogen atmosphere. The resulting mixture was stirred at room temperature for 14 hours. The desired product was detected via LCMS. The resulting mixture was concentrated under reduced pressure. The residue was purified by flash chromatography on silica gel column using 0-50% of EtOAc in petroleum ether as eluent to provide methyl (1r,3r)-3-(benzo[d]oxazol-5-yloxy)cyclobutane-1-carboxylate as a pink solid (122.0 mg, 66.6%). LCMS (ESI) m/z 248.1, [M+H]⁺.

### Step 2: ((1r,3r)-3-benzo[d]oxazol-5-yloxy)cyclobutyl)methanol

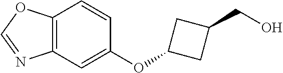 [SMILES: OC[C@H]1C[C@H](Oc2ccc3ocnc3c2)C1]

To a stirred solution of methyl (1r,3r)-3-(benzo[d]oxazol-5-yloxy)cyclobutane-1-carboxylate (80.1 mg; 0.324 mmol; 1.00 eq.) in MeOH (5 mL) was added NaBH₄ (213.6 mg, 5.647 mmol, 17.42 eq.) in portions at 0° C. under nitrogen atmosphere. The desired product was detected via LCMS. The resulting mixture was quenched by water (1 mL), concentrated under reduced pressure. The residue was purified by flash chromatography on silica gel column using 0-50% of EtOAc in petroleum ether as eluent to provide ((1r,3r)-3-(benzo[d]oxazol-5-yloxy)cyclobutyl)methanol as a white solid (40.0 mg, 56.3%). LCMS (ESI) m/z 220.1, [M+H]⁺.

### Step 3: 5-(1r,3r)-3-(methoxymethyl)cyclobutoxy)benzo[d]oxazole

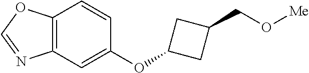 [SMILES: COC[C@H]1C[C@H](Oc2ccc3ocnc3c2)C1]

To a stirred solution of ((1r,3r)-3-(benzo[d]oxazol-5-yloxy)cyclobutyl)methanol (30.0 mg; 0.137 mmol; 1.00 eq.) in THF (3 mL) was added NaH (60%) (15.9 mg; 0.667 mmol; 4.87 eq.) at 0° C. under nitrogen atmosphere. After stirring at room temperature for 0.5 hour, to the above solution was added CH₃I (39.0 mg; 0.275 mmol; 2.01 eq.) dropwise. The resulting mixture was stirred at room temperature for an additional 0.5 hour. The desired product was detected via LCMS. The resulting mixture was quenched by saturated NH₄Cl solution (10 mL), extracted with EtOAc (3×10 mL). The combined organic phases were dried over anhydrous Na₂SO₄. After filtration, the filtrate was concentrated under reduced pressure. The residue was purified by flash chromatography on silica gel column using 30-50% of EtOAc in petroleum ether as eluent to provide 5-(1r,3r)-3-(methoxymethyl)cyclobutoxy)benzo[d]oxazole as a white solid (15.0 mg, 46.9%). LCMS (ESI) m/z 234.1, [M+H]⁺.

### Step 4: N-(5-(5-((1r,3r)-3-(methoxymethyl)cyclobutoxy)benzo[d]oxazol-211)-8-(methylamino)-2,7-naphthyridin-3-yl)cyclopropanecarboxamide

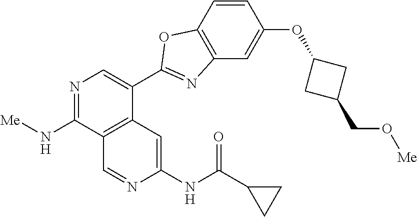 [SMILES: CNc1ncc(-c2nc3cc(O[C@H]4C[C@H](COC)C4)ccc3o2)c2cc(NC(=O)C3CC3)ncc12]

N-(5-(5-((1r,3r)-3-(methoxymethyl)cyclobutoxy)benzo[d]oxazol-2-yl)-8-(methyl amino)-2,7-naphthyridin-3-yl)cyclopropanecarboxamide was synthesized using a similar procedure that was previously described in Example 345 by using N-(5-bromo-8-(methylamino)-2,7-naphthyridin-3-yl)cyclopropanecarboxamide and 54(1r,3r)-3-(methoxymethyl)cyclobutoxy)benzo[d]oxazole as the starting material. LCMS (ESI) m/z 474.2. ¹H NMR (400 MHz, DMSO-d₆) δ 11.04 (s, 1H), 9.58 (s, 9.44 (s, 1H), 8.88 (s, 1H), 8.69-8.61 (m, 1H), 7.61 (d, J=8.8 Hz, 1H), 7.09 (d, 2.4 Hz, 1H), 6.92-6.85 (m, 1H), 4.96-4.85 (m, 1H), 3.45 (d, J=6.9 Hz, 2H), 3.31 (s, 3H), 3.09 (d, J=4.4 Hz, 3H), 2.39-1.94 (m, 6H), 0.96-0.82 (m, 4H).

### Example 349: Synthesis of N-(8-(methylamino)-5-(5-((tetrahydro-1H-pyrrolizin-7a(5H)-yl)methoxy)benzo[d]oxazol-2-yl)-2,7-naphthyridin-3-yl)cyclopropanecarboxamide

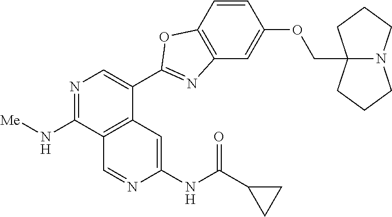 [SMILES: CNc1ncc(-c2nc3cc(OCC45CCCN4CCC5)ccc3o2)c2cc(NC(=O)C3CC3)ncc12]

### Step 1: 5-tetrahydro-1H-pyrrolizin-7a(5H)-yl)methoxy)benzo[d]oxazole

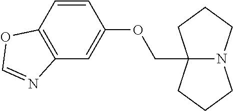 [SMILES: c1nc2cc(OCC34CCCN3CCC4)ccc2o1]

To a solution of benzo[d]oxazol-5-ol (300.0 mg; 2.220 mmol; 1.00 eq.) and (tetrahydro-1H-pyrrolizin-7a(5H)-yl)methanol (470.0 mg; 3,328 mmol; 1.50 eq.) in THF (10 mL) were added PPh₃ (634.0 mg; 2.417 mmol; 1.09 eq.) and DIAD (448.0 mg; 2.216 mmol; 1.00 eq.). The reaction was stirred at 50° C. for 14 hours under nitrogen atmosphere. The desired product was detected via LCMS. The solvent was concentrated under vacuum. The residue was purified by flash chromatography on pre-packed C18 column using 20-70% of MeI N in water (10 mmol/L NH₄HCO₃) to provide 5-((tetrahydro-1H-pyrrolizin-7a(5H)-yl)methoxy)benzo[d]oxazole as an off-white solid (145.0 mg, 25.2%). LCMS (ESI) m/z 259.1, [M+H]⁺.

### Step 2: N-(8-(methylamino)-5-(5-((tetrahydro-1H-pyrrolizin-7a(5H)-yl)methoxy)benzo[d]oxazol-2-yl)-2,7-naphthyridin-3-yl)cyclopropanecarboxamide

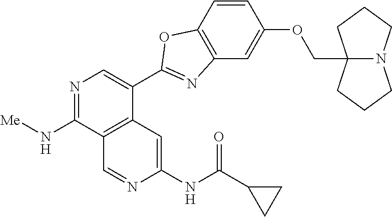 [SMILES: CNc1ncc(-c2nc3cc(OCC45CCCN4CCC5)ccc3o2)c2cc(NC(=O)C3CC3)ncc12]

N-(8-(methylamino)-5-(5-((tetrahydro-1H-pyrrolizin-7a(5H)-yl)methoxy)benzo[d]oxazol-2-yl)-2,7-naphthyridin-3-yl)cyclopropanecarboxamide was synthesized using a similar procedure that was previously described in Example 345, by using N-(5-bromo-8-(methylamino)-2,7-naphthyridin-3-yl)cyclopropanecarboxamide and 5-((tetrahydro-1H-pyrrolizin-7a(5H)-yl)methoxy)benzo[d]oxazole as the starting material. LCMS (ESI) m/z 499.1, [M+H]⁺. ¹H NMR (400 MHz, DMSO-d₆) δ 11.03 (s, 1H), 9.67 (s, 1H), 9.44 (s, 1H), 8.89 (s, 1H), 8.68-8.60 (m, 1H), 7.60 (d, J=8.8 Hz, 1H), 7.24 (d, J=2.4 Hz, 1H), 6.95 (dd, J=8.8, 2.4 Hz, 1H), 3.72 (s, 2H), 3.09 (d, J===4.4 Hz, 3H), 3.01-2.90 (m, 2H), 2.62-2.51 (m, 2H), 2.16-2.05 (m, 1H), 2.00-1.88 (m, 2H), 1.88-1.70 (m, 4H), 1.68-1.52 (m, 2H), 0.96-0.78 (m, 4H).

### Example 350: Synthesis of (S)—N-(5-(5-((1-methoxypropan-2-yl)oxy)benzo[d]oxazol-2-yl)-8-(methylamino)-2,7-naphthyridin-3-yl)cyclopropanecarboxamide

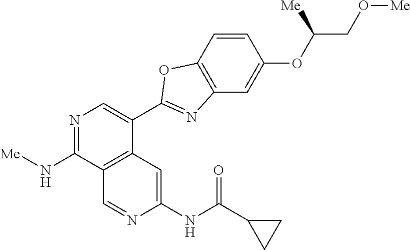 [SMILES: CNc1ncc(-c2nc3cc(O[C@@H](C)COC)ccc3o2)c2cc(NC(=O)C3CC3)ncc12]

### Step 1: (S)-5-((1-methoxypropan-2-yl)oxy)benzo[d]oxazole

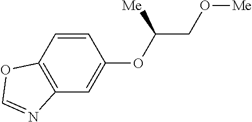 [SMILES: COC[C@H](C)Oc1ccc2ocnc2c1]

To a mixture of benzo[d]oxazol-5-ol (200.0 mg; 1.480 mmol; 1.00 eq.), PPh₃ (427.0 mg; 1.628 mmol; 1.10 eq.) and (R)-1-methoxypropan-2-ol (106.7 mg, 1.184 mmol; 0.80 eq.) in THF (8 mL) was added DIAD (300.0 mg; 1.480 mmol; 1.00 eq.) at 0° C. under nitrogen atmosphere. The reaction was stirred at room temperature for 3 hours. The desired product was detected via LCMS. The resulting mixture was concentrated under reduced pressure. The residue was purified by flash chromatography on silica gel column using 10-30% of EtOAc in petroleum ether as eluent to provide (S)-5-((1-methoxypropan-2-yl)oxy)benzo[d]oxazole as yellow solid (243.0 mg, 79.2%). LCMS (ESI) m/z 208.1, [M+H]⁺.

### Step 2: (S)—N-(5-(5-((1-methoxypropan-2-yl)oxy)benzo[d]oxazol-2-yl)-8-(methylamino)-2,7-naphthyridin-3-yl)cyclopropanecarboxamide

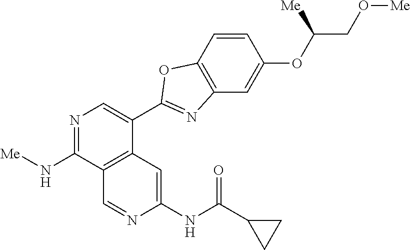 [SMILES: CNc1ncc(-c2nc3cc(O[C@@H](C)COC)ccc3o2)c2cc(NC(=O)C3CC3)ncc12]

(S)—N-(5-(5-((1-methoxypropan-2-yl)oxy)benzo[d]oxazol-2-yl-8-(methylamino)-2,7-naphthyridin-3-yl)cyclopropanecarboxamide was synthesized using a similar procedure that was previously described in Example 345 by using N-(5-bromo-8-(methylamino)-2,7-naphthyridin-3-yl)cyclopropanecarboxamide and (S)-5-(((1-methoxypropan-2-yl)oxy)benzo[d]oxazole as the starting material. LCMS (ESI) m/z 448.2, [M+H]⁺. ¹H NMR (400 MHz, DMSO-d₆) δ 11.04 (s, 1H), 9.62 (s, 1H), 944 (s, 1H), 8.89 (s, 1H), 8.68-8.60 (m, 1H), 7.60 (d, 8.8 Hz, 1H), 7.31 (d, J=2.4 Hz, 1H), 6.96 (dd, J=8.8, 2.4 Hz, 1H), 4.75-4.64 (m, 1H), 3.61-3.44 (m, 2H), 3.32 (s, 3H), 3.09 (d, 4.4 Hz, 3H), 2.16-2.05 (m, 1H), 1.26 (d, J===6.2 Hz, 3H), 0.96-0.82 (m, 4H).

### Example 351: Synthesis of (R)—N-(5-(5-((1-methoxypropan-2-yl)oxy)benzo[d]oxazol-2H)-8-(methylamino)-2,7-naphthyridin-3-yl)cyclopropanecarboxamide

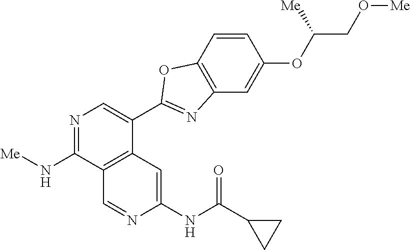 [SMILES: CNc1ncc(-c2nc3cc(O[C@H](C)COC)ccc3o2)c2cc(NC(=O)C3CC3)ncc12]

### Step 1: (R)-5-((1-methoxypropan-2-yl)oxy)benzo[d]oxazole

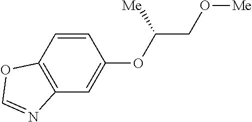 [SMILES: COC[C@@H](C)Oc1ccc2ocnc2c1]

To a solution of benzo[d]oxazol-5-ol (200.0 mg; 1.480 mmol; 1.00 eq.), (S)-1-methoxypropan-2-ol (106.7 mg; 1.184 mmol; 0.80 eq.) and PPh₃ (427.0 mg; 1.628 mmol; 1.10 eq.) in THF (10 mL) was added DIAD (299 mg; 1,479 mmol; 1.00 eq.) at 0° C. under nitrogen atmosphere. The reaction was stirred at room temperature for 3 hours. The desired product was detected via LCMS. The resulting mixture was concentrated under vacuum. The residue was purified by flash chromatography on silica gel column using 0-30% of EtOAc in petroleum ether as eluent to provide (R)-5-((1-methoxypropan-2-yl)oxy)benzo[d]oxazole as a pink liquid (171.0 mg, 55.7%). LCMS (ESI) m/z 208.1, [M+H]⁺.

### Step 2: (R)—N-(5-(5-((1-methoxypropan-2-yl)oxy)benzo[d]oxazol-2-yl)-8-(methylamino)-2,7-naphthyridin-3-yl)cyclopropanecarboxamide

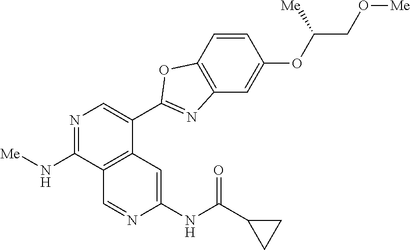 [SMILES: CNc1ncc(-c2nc3cc(O[C@H](C)COC)ccc3o2)c2cc(NC(=O)C3CC3)ncc12]

(R)—N-(5-(5-((1-methoxypropan-2-yl)oxy)benzo[d]oxazol-2-yl)-8-(methylamino)-2,7-naphthyridin-3-yl)cyclopropanecarboxamide was synthesized using a similar procedure that was previously described in Example 345 by using N-(5-bromo-8-(methylamino)-2,7-naphthyridin-3-yl)cyclopropanecarboxamide and (R)-5-((1-methoxypropan-2-yl)oxy)benzo[d]oxazole as the starting material. LCMS (ESI) m/z 448.2, [M+H]⁺. ¹H NMR (400 MHz, DMSO-d₆) δ 11.04 (s, 1H), 9.62 (s, 1H), 9.44 (s, 1H), 8.89 (s, 1H), 8.68-8.60 (m, 1H), 7.60 (d, J=8.8 Hz, 1H), 7.31 (d, J=2.4 Hz, 1H), 6.96 (dd, 8.8, 2.4 Hz, 1H), 4.75-4.64 (m, 1H), 3.57-3.45 (m, 2H), 3.35 (s, 3H), 3.09 (d, J=4.4 Hz, 3H), 2.16-2.05 (m, 1H), 1.26 (d, J=6.2 Hz, 3H), 0.96-0.81 (m, 4H).

### Example 352: Synthesis of N-(5-(5-(2-methyl-2-morpholinopropoxy)benzo[d]oxazol-2-yl)-8-(methylamino)-2,7-naphthyridin-3-yl)cyclopropanecarboxamide

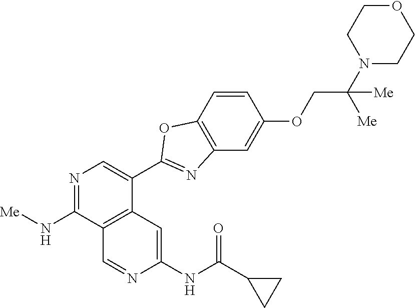 [SMILES: CNc1ncc(-c2nc3cc(OCC(C)(C)N4CCOCC4)ccc3o2)c2cc(NC(=O)C3CC3)ncc12]

### Step 1: 5-(2-methyl-2-morpholinopropoxy)benzo[d]oxazole

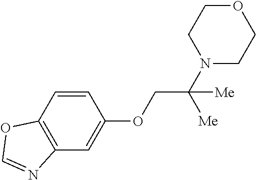 [SMILES: CC(C)(COc1ccc2ocnc2c1)N1CCOCC1]

To a solution of benzo[d]oxazol-5-ol (100.0 mg; 0.740 mmol; 1.00 eq.), 2-methyl-2-morpholinopropan-1-ol (94.3 mg; 0.592 mmol; 0.80 eq.) and PPh₃ (213.5 mg; 0.814 mmol, 1.10 eq.) in THF (5 mL) was added MAD (149.7 mg; 0.740 mmol; 1.00 eq.) drop-wise at 0° C. under nitrogen atmosphere. The resulting mixture was stirred at 50° C. for 14 hours. The resulting mixture was diluted with EtOAc (30 mL) and washed with brine (2×10 mL). The organic layer was dried over anhydrous Na₂SO₄. After filtration, the filtrate was concentrated under reduced pressure. The residue was purified by flash chromatography on silica gel column using 15-50% of EtOAc in petroleum ether as eluent to provide 5-(2-methyl-2-morpholinopropoxy)benzo[d]oxazole as a white solid (150.0 mg, 73.3%). LCMS (ESI) m/z 277.1, [M+H]⁺.

### Step 2: N-(5-(5-(2-methyl-2-morpholinopropoxy)benzo[d]oxazol-2-yl)-8-(methylamino)-2,7-naphthyridin-3-yl)cyclopropanecarboxamide

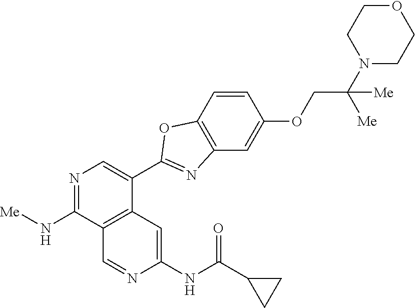 [SMILES: CNc1ncc(-c2nc3cc(OCC(C)(C)N4CCOCC4)ccc3o2)c2cc(NC(=O)C3CC3)ncc12]

A mixture of 5-(2-methyl-2-morpholinopropoxy)benzo[d]oxazole (50.3 mg; 0.182 mmol; 1.17 eq.), N-(5-bromo-8-(methylamino)-2,7-naphthyridin-3-yl)cyclopropanecarboxamide (50.0 mg; 0.156 mmol; 1.00 eq.), Pd(PPh₃)₄ (10.8 mg; 0.009 mmol; 0.06 eq.) and Cs₂CO₃ (152.2 mg; 0.467 mmol; 3.00 eq.) in DMF (5 mL) was stirred at 90° C. for 14 hours under nitrogen atmosphere. After the reaction was completed, the resulting mixture was diluted with EtOAc (30 mL). The organic phase was washed with brine (2×10 mL), dried over anhydrous Na₂SO₄, filtered, and concentrated under reduced pressure. The residue was purified by flash chromatography on silica gel column using 0-10% of MeOH in CH₂Cl₂ as eluent to provide N-(5-(5-(2-methyl-2-morpholinopropoxy)benzo[d]oxazol-2-yl)-8-(methylamino)-2,7-naplathyridin-3-yl)cyclopropanecarboxamide as a yellow solid (6.7 mg, 8.2%). LCMS (ESI) m/z 517.2, [M+H]⁺. ¹H NMR (400 MHz, DMSO-d₆) δ 11.05 (s, 1H), 9.64 (s, 1H), 9.45 (s, 1H), 8.90 (s, 1H), 8.70-8.61 (m, 1H), 7.62 (d, J=8.4 Hz, 1H), 7.32 (d, J=2.4 Hz, 1H), 7.09-6.98 (m, 1H), 3.69-3.58 (m, 4H), 3.09 (d, J=4.4 Hz, 3H), 2.67-2.53 (m, 6H), 2.15-2.06 (m, 1H), 1.28 (s, 6H), 0.93-0.84 (m, 4H).

### Example 353: Synthesis of N-(8-(methylamino)-5-(5-(2-(methylsulfinyl)ethoxy) benzo[d]oxazol-2-yl)-2,7-naphthyridin-3-yl)cyclopropanecarboxamide

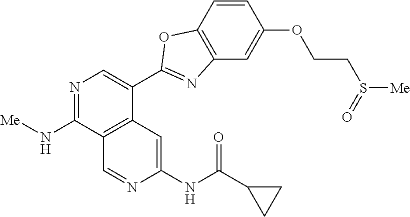 [SMILES: CNc1ncc(-c2nc3cc(OCCS(C)=O)ccc3o2)c2cc(NC(=O)C3CC3)ncc12]

### Step 1: 5-(2-(methylthio)ethoxy)benzo[d]oxazole

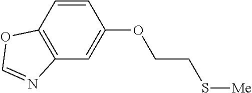 [SMILES: CSCCOc1ccc2ocnc2c1]

To a solution of benzo[d]oxazol-5-ol (200.1 mg; 1.481 mmol; 0.80 eq.), 2-(methylthio)ethan-1-ol (170.6 mg; 1.851 mmol; 1.00 eq.) and PPh₃ (534.1 mg; 2.036 mmol; 1.10 eq.) in THF (5 mL) was added diisopropyl (E)-diazene-1,2-dicarboxylate (374.3 mg, 1.851 mmol, 1.00 eq.) at 0° C. under nitrogen atmosphere. The resulting solution was stirred at 50° C. for 5 hours under nitrogen atmosphere. The desired product was detected via LCMS. The reaction was concentrated under vacuum. The residue was purified by flash chromatography on silica gel column using 0-15% of Maki: in CH₂Cl₂ as eluent to provide 5-(2-(methylthio)ethoxy)benzo[d]oxazole as a white solid (200.0 mg, 49.5%). LCMS (ESI) m/z 210.0, [M+H]⁺.

### Step 2: N-(8-(methylamino)-5-(5-(2-(methylthio)ethoxy)benzo[d]oxazol-2-yl)-2,7-naphthyridin-3-yl)cyclopropanecarboxamide

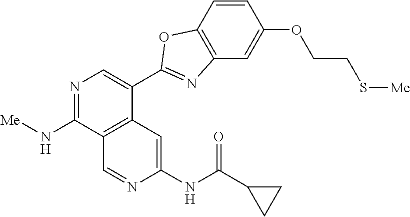 [SMILES: CNc1ncc(-c2nc3cc(OCCSC)ccc3o2)c2cc(NC(=O)C3CC3)ncc12]

A stirred mixture of 5-(2-(methylthio)ethoxy)benzo[d]oxazole (78.2 mg; 0.373 mmol; 1.20 eq.), N-(5-bromo-8-(methyl amino)-2,7-naphthyridin-3-yl)cyclopropanecarboxamide (100.0 mg; 0.311 mmol; 1.00 eq.), Cs₂CO₃ (304.3 mg; 0.934 mmol; 3.00 eq.) and Pd(PPh₃)₄ (350.9 mg; 0.031 mmol; 0.10 eq.) in DMF (10 mL) was stirred at 110° C. for 16 hours under nitrogen atmosphere. The reaction mixture was cooled to room temperature and concentrated under reduced pressure. The residue was purified by flash chromatography on silica gel column using 2-10% of MeOH in CH₂Cl₂ to afford N-(8-(methylamino)-5-(5-(2-(methylthio)ethoxy)benzo[d]oxazol-2-yl)-2,7-naphthyridin-3-yl)cyclopropanecarboxamide as a yellow solid (100.0 mg, 67.9%). LCMS (ESI) m/z 450.1, [M+H]⁺.

### Step 3: N-(8-(methylamino)-5-(5-(2-(methylsulfinyl)ethoxy)benzo[d]oxazol-2-yl)-2,7-naphthyridin-3-yl)cyclopropanecarboxamide

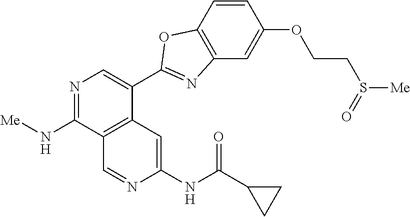 [SMILES: CNc1ncc(-c2nc3cc(OCCS(C)=O)ccc3o2)c2cc(NC(=O)C3CC3)ncc12]

To a solution of N-(8-(methylamino)-5-(5-(2-(methy thio)ethoxy)benzo[d]oxazol-2-yl)-2,7-naphthyridin-3-yl)cyclopropanecarboxamide (40.0 mg, 0.089 mmol; 1.00 eq.) in CH₂Cl₂ (10 mL) was added 3-chlorobenzoperoxoic acid (46.1 mg; 0.267 mmol; 3.00 eq.) at 0° C. The mixture was stirred at room temperature for 3 hours. The desired product was detected via LCMS. The precipitated solids were collected by filtration and washed with CH₂Cl₂ (3×10 mL). The solids were purified by flash chromatography on silica gel column using 0-15% of MeOH in CH₂Cl₂ as eluent to provide N-(8-(methylamino)-5-(5-(2-(methylsulfinyl)ethoxy)benzo[d]oxazol-2-yl)-2,7-naphthyridin-3-yl)cyclopropanecarboxamide as a yellow solid (13.0 mg, 31.1%), LCMS (ESI) m/z 466.1, [M+H]⁺. ¹H NMR (400 MHz, DMSO-d₆) δ 11.04 (s, 1H), 9.65 (s, 1H), 9.44 (s, 1H), 8.90 (s, 1H), 8.70-8.61 (m, 1H), 7.64 (d, J=8.8 Hz, 1H), 7.36 (d, J=2.4 Hz, 1H), 7.04-6.97 (m, 1H), 4.54-4.38 (m, 2H), 3.32-3.28 (m, 1H), 3.12-3.07 (m, 4H), 2.67 (s, 3H), 2.14-2.07 (m, 1H), 0.94-0.90 (m, 2H), 0.89-0.83 (m, 2H).

### Example 354: Synthesis of N-(8-(methylamino)-5-(5-(2-(methylsulfonyl)ethoxy)benzo[d]oxazol-2-yl)-2,7-naphthyridin-3-yl)cyclopropanecarboxamide

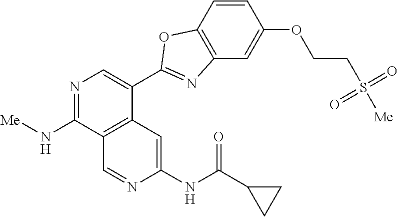 [SMILES: CNc1ncc(-c2nc3cc(OCCS(C)(=O)=O)ccc3o2)c2cc(NC(=O)C3CC3)ncc12]

To a mixture of N-(8-(methylamino)-5-(5-(2-(methylsulfinyl)ethoxy)benzo[d]oxazol-2-yl)-2,7-naphthyridin-3-yl)cyclopropanecarboxamide (Example 353) (80.0 mg; 0.172 mmol; 1.00 eq.) in NMP (5 mL) was added 3-chlorobenzoperoxoic acid (29.6 mg; 0.172 mmol; 1.00 eq.) at 0° C. The mixture was stirred at room temperature for 3 hours. The desired product was detected via LCMS. The reaction was quenched with saturated-Na₂S₂O₃ aqueous solution (5 mL) at 0° C. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by flash chromatography on pre-packed C18 column using 10-80% of MeCN in water (10 mmol/L NH₄HCO₃) as eluent to provide N-(8-(methylamino)-5-(5-(2-(methylsulfonyl)ethoxy)benzo[d]oxazol-2-yl)-2,7-naphthyridin-3-yl)cyclopropanecarboxamide as a yellow solid (28.0 mg, 33.0%). LCMS (ESI) m/z 482.1, [M+H]⁺. ¹H NMR (400 MHz, DMSO-d₆) δ 11.03 (s, 1H), 9.56 (s, 1H), 9.44 (s, 1H), 8.90 (s, 1H), 8.71-8.61 (m, 1H), 7.65 (d, J=8.8 Hz, 1H), 7.38 (d, J=2.4 Hz, 1H), 7.06-7.00 (m, 1H), 4.51-4.42 (m, 2H), 3.70-3.62 (m, 2H), 3.17-3.07 (m, 6H), 2.16-2.07 (m, 1H), 0.95-0.83 (m, 4H).

### Example 355: Synthesis of N-(8-(methylamino)-5-(6-morpholinobenzo[d]oxazol-2-yl)-2,7-naphthyridin-3-yl)cyclopropanecarboxamide

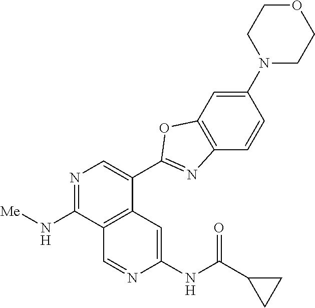 [SMILES: CNc1ncc(-c2nc3ccc(N4CCOCC4)cc3o2)c2cc(NC(=O)C3CC3)ncc12]

### Step 1: 2-methyl-6-morpholinobenzo[d]oxazole

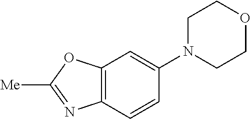 [SMILES: Cc1nc2ccc(N3CCOCC3)cc2o1]

A solution of 6-bromo-2-methyl-1,3-benzoxazole (1.02 g; 4.716 mmol; 1.00 eq.), morpholine (657.4 mg; 7.546 mmol; 1.60 eq.), BrettPhos Pd G₃ (769.4 mg; 0.848 mmol; 0.18 eq.), BrettPhos (227.8 mg; 0.414 mmol; 0.09 eq.) and Cs₂CO₃ (4.00 g; 12.276 mmol; 2.60 eq.) in 1,4-dioxane (34 mL) was stirred at 100° C. for 14 hours under nitrogen atmosphere. The desired product was detected via LCMS. The resulting mixture was concentrated under reduced pressure. The residue was purified by flash chromatography on silica gel column using 30-45% of EtOAc in petroleum ether as eluent to provide 2-methyl-6-morpholinobenzo[d]oxazole as a yellow solid (631.8 mg, 61.3%), LCMS (ESI) m/z 219.1, [M+H]⁺.

### Step 2: 2-amino-5-morpholinophenol

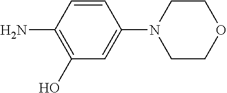 [SMILES: Nc1ccc(N2CCOCC2)cc1O]

To a solution of 2-methyl-6-(morpholin-4-yl)-1,3-benzoxazole (630.0 mg; 2.887 mmol; 1.00 eq.) in EtOH (20 mL) was added a solution of HCl (4 M in water, 6 mL). The resulting solution was stirred at 100° C. for 4 hours. The desired product was detected via LCMS. The resulting mixture was concentrated under reduced pressure to afford 2-amino-5-morpholinophenol as a white solid (630.0 mg, crude). The crude product was used in the next step directly without further purification, LCMS (ESI) m/z 195.1, [M+H]⁺.

### Step 3: 6-morpholinobenzo[d]oxazole

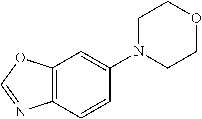 [SMILES: c1nc2ccc(N3CCOCC3)cc2o1]

A solution of 2-amino-5-(morpholin-4-yl)phenol (630.0 mg; 3.244 mmol; 1.00 eq.) in trimethyl orthoformate (20 mL) was stirred at 100° C. overnight under nitrogen atmosphere. The desired product was detected via LCMS. The resulting mixture was concentrated under reduced pressure. The residue was purified by flash chromatography on silica gel column using 30-40% of EtOAc in petroleum ether as eluent to provide 6-morpholinobenzo[d]oxazole as a yellow solid (563.5 mg, 85.0%). LCMS (ESI) m/z 205.1, [M+H]⁺.

### Step 4: N-(8-(methylamino)-5-(6-morpholinobenzo[d]oxazol-2-yl)-2,7-naphthyridin-3-yl)cyclopropanecarboxamide

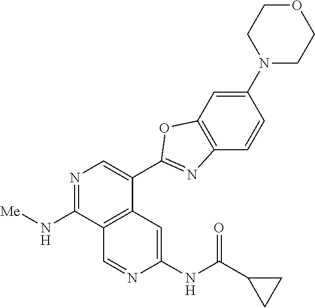 [SMILES: CNc1ncc(-c2nc3ccc(N4CCOCC4)cc3o2)c2cc(NC(=O)C3CC3)ncc12]

N-(8-(methylamino)-5-(6-morpholinobenzo[d]oxazol-2-yl)-2,7-naphthyridin-3-yl)cyclopropanecarboxamide was synthesized using a similar procedure that was previously described in Example 345, by using N-(5-bromo-8-(methylamino)-2,7-naphthyridin-3-yl)cyclopropanecarboxamide and 6-morpholinobenzo[d]oxazole as the starting material. LCMS (ESI) m/z 445.2, [M+H]⁺. ¹H NMR (400 MHz, DMSO-d₆) δ 11.00 (s, 1H), 9.64 (s, 1H), 9.43 (s, 1H), 8.83 (s, 1H), 8.58-8.52 (m, 1H), 7.59 (d, J=8.8 Hz, 1H), 7.27 (d, J=2.4 Hz, 1H), 7.05 (dd, =8.8, 2.4 Hz, 1H), 3.82-3.74 (m, 4H), 3.22-3.15 (m, 4H), 3.08 (d, 4.4 Hz, 3H), 2.14-2.05 (m, 1H), 0.95-0.81 (m, 4H).

### Example 356: Synthesis of N-(8-(methylamino)-5-(oxazolo[4,5-c]pyridin-2-yl)-2,7-naphthyridin-3-yl)cyclopropanecarboxamide

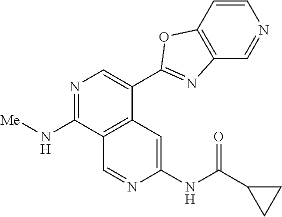 [SMILES: CNc1ncc(-c2nc3cnccc3o2)c2cc(NC(=O)C3CC3)ncc12]

### Step 1: methyl 6-(cyclopropanecarboxamido)-1-(methylamino)-2,7-naphthyridine-4-carboxylate

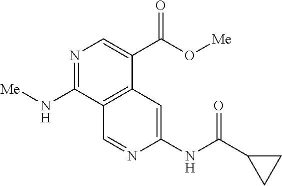 [SMILES: CNc1ncc(C(=O)OC)c2cc(NC(=O)C3CC3)ncc12]

To a solution of N-(5-bromo-8-(methylamino)-2,7-naphthyridin-3-yl)cyclopropanecarboxamide (3.00 g; 9.341 mmol; 1.00 eq.) in a mixture solvent of DMSO/MeOH (2:1, 27 mL) was added Pd(dppf)Cl₂·CH₂Cl₂ (760.9 mg; 0.934 mmol; 0.10 eq.) and Et₃N (2.84 g; 28.065 mmol; 3.00 eq.) in a pressure tank. The mixture was stirred at 130° C. overnight under carbon monoxide atmosphere (20 atm). The reaction mixture was cooled to room temperature. The desired product was detected via LCMS. The reaction solution was diluted with EtOAc, (500 mL), washed with brine (3×50 mL). The organic layer was dried over Na₂SO₄. After filtration, the filtrate was concentrated under reduced pressure. The residue was purified by flash chromatography on silica gel column using 60-90% of EtOAc in petroleum ether as eluent to provide methyl 6-cyclopropaneamido-1-(methylamino)-2,7-naphthyridine-4-carboxylate as a yellow solid (2.80 g, 99.8%). LCMS (ESI) m/z 301.1, [M+H]⁺.

### Step 2: 66(cyclopropanecarboxamido)-1-(methylamino)-2,7-naphthyridine-4-carboxylic acid

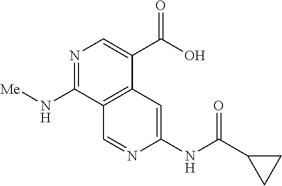 [SMILES: CNc1ncc(C(=O)O)c2cc(NC(=O)C3CC3)ncc12]

To a solution of methyl 6-cyclopropaneamido-1-(methylamino)-2,7-naphthyridine-4-carboxylate (1.00 g; 3.330 mmol; 1.00 eq.) in THF (60 mL) was added a solution of LiOH (280.0 mg; 11.691 mmol; 3.51 eq.) in water (20 mL) at 0° C. The mixture was stirred at room temperature overnight under nitrogen atmosphere. LC-MS indicated that the reaction was complete. The solvent was removed under reduced pressure, the mixture was acidified to pH=5 using a solution of HCl (1N). Then the precipitated product was collected by filtration and washed with water. The solids were dried under vacuum to provide 6-cyclopropaneamido-1-(methylamino)-2,7-naphthyridine-4-carboxylic acid as a brown solid (840 mg, 88.1%). LCMS (ESI) m/z 287.1, [M+H]⁺.

### Step 3: 6-(cyclopropanecarboxamido)-N-(4-hydroxypyridin-3-yl)-1-(methylamino)-2,7-naphthyridine-4-carboxamide

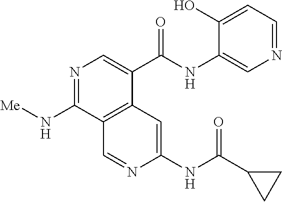 [SMILES: CNc1ncc(C(=O)Nc2cnccc2O)c2cc(NC(=O)C3CC3)ncc12]

To a solution of 6-(cyclopropanecarboxamido)-1-(methylamino)-2,7-naphthyridine-4-carboxylic acid (250.0 mg; 0.872 mmol; 1.00 eq.) and PyAOP (550.0 mg; 1.055 mmol; 1.21 eq.) in DMA (3 mL) were added 3-aminopyridin-4-01 (100.0 mg; 0.907 mmol; 1.04 eq.), DIPEA (337.5 mg; 2.612 mmol; 2.99 eq.) and DMAP (12.5 mg; 0.101 mmol; 0.12 eq.). The mixture was stirred at room temperature for 1 hour. The desired product was detected via LCMS. The resulting mixture was purified by flash chromatography on pre-packed C18 column using 20-60% of MeCN in water (10 mmol/L NH₄HCO₃) as eluent to afford 6-(cyclopropanecarboxamido)-N-(4-hydroxypyridin-3-yl)-1-(methylamino)-2,7-naphthyridine-4-carboxamide as a off-white solid (200.0 mg, 60.5%). LCMS (ESI) m/z 379.1, [M+H]⁺.

### Step 4: N-(8-(methylamino)-5-(oxazolo[4,5-c]pyridin-2-yl)-2,7-naphthyridin-3-yl)cyclopropanecarboxamide

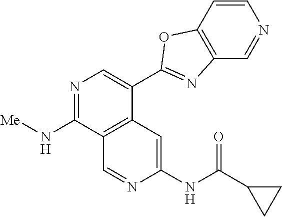 [SMILES: CNc1ncc(-c2nc3cnccc3o2)c2cc(NC(=O)C3CC3)ncc12]

A solution of 6-(cyclopropanecarboxamido)-N-(4-hydroxypyridin-3-yl)-1-(methylamino)-2,7-naphthyridine-4-carboxamide (200.0 mg; 0.529 mmol; 1.00 eq.) and P₂O₅(400.0 mg; 2.818 mmol; 5.33 eq.) in NMP (8 mL) was stirred at 110° C. for 1 hour under nitrogen atmosphere. The desired product was detected via LCMS. The mixture was purified by flash chromatography on pre-packed C18 column using 20-50% of MeCN in water (10 mmol/L, NH₄HCO₃) as eluent to afford N-(8-(methylamino)-5-(oxazolo[4,5-c]pyridin-2-yl)-2,7-naphthyridin-3-yl)cyclopropanecarboxamide as an off-white solid (12.2 mg, 6.2%), LCMS (ESI) m/z 361.1, [M+H]⁺. ¹H NMR (400 MHz, DMSO-d₆) δ 11.07 (s, 1H), 9.68 (s, 1H), 9.46 (s, 1H), 9.04 (d, J=1.0 Hz, 1H), 8.98 (s, 1H), 8.80-8.73 (m, 1H), 8.56 (d, J=5.2 Hz, 1H), 7.84 (dd, J=5.2, 1.0 Hz, 1H), 3.11 (d, J=4.4 Hz, 3H), 2.11-2.08 (m, 1H), 0.97-0.84 (m, 4H).

### Example 357: Synthesis of N-(8-(methylamino)-5-(oxazolo[5,4-c]pyridin-2-yl)-2,7-naphthyridin-3-yl)cyclopropanecarboxamide

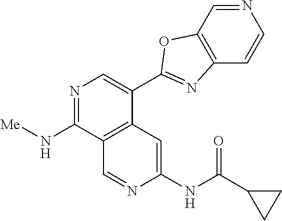 [SMILES: CNc1ncc(-c2nc3ccncc3o2)c2cc(NC(=O)C3CC3)ncc12]

### Step 1: 6-(cyclopropanecarboxamide-N-(3-iodopyridin-4-yl)-1-(methylamino)-2,7-naphthyridine-4-carboxamide

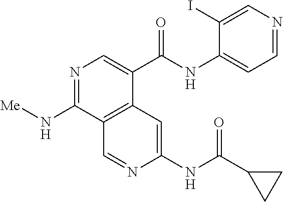 [SMILES: CNc1ncc(C(=O)Nc2ccncc2I)c2cc(NC(=O)C3CC3)ncc12]

To a stirred solution of 6-(cyclopropanecarboxamido)-1-(methylamino)-2,7-naphthyridine-4-carboxylic acid (Example 356, step 2) (300.1 mg; 1.048 mmol; 1.00 eq.) and 3-iodopyridin-4-amine (138.2 mg; 1.252 mmol; 1.20 eq.) in DMA (5 mL) were added EDCI (300.2 mg; 1.565 mmol, 1.50 eq.) and DMAP (192.3 mg; 1.572 mmol, 1.50 eq.). The resulting mixture was stirred at room temperature overnight. The desired product was detected via LCMS. The mixture was purified by flash chromatography on pre-packed. C18 column using 30-50% of MeCN in water (10 mmol/L, NH₄HCO₃) as eluent to provide 6-(cyclopropanecarboxamido)-N-(3-iodopyridin-4-yl)-1-(methylamino)-2,7-naphthyridine-4-carboxamide as an off-white solid (120.2 mg, 30.2%). LCMS (JEST) m/z 489.0, [M+H]⁺.

### Step 2: N-(8-(methylamino)-5-(oxazolo[5,4-c]pyridin-2-yl)-2,7-naphthyridin-3-3/1)cyclopropanecarboxamide

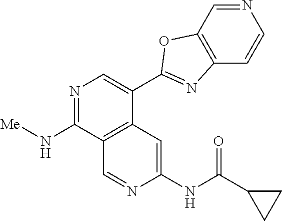 [SMILES: CNc1ncc(-c2nc3ccncc3o2)c2cc(NC(=O)C3CC3)ncc12]

To a stirred solution of 6-(cyclopropanecarboxamido)-N-(3-iodopyridin-4-yl)-1-(methylamino)-2,7-naphthyridine-4-carboxamide (30.1 mg; 0.061 mmol; 1.00 eq.) and Cull (2.4 mg; 0.013 mmol; 0.20 eq.) in 1,4-dioxane (1 mL) were added 1.10-phenanthroline (2.1 mg; 0.012 mmol; 0.20 eq.) and Cs₂CO₃ (40.2 mg; 0.123 mmol; 2.00 eq.). The resulting mixture was stirred at 100° C. for 14 hours under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide N-(8-(methylamino)-5-(oxazolo[5,4-c]pyridin-2-yl)-2,7-naphthyridin-3-yl)cyclopropanecarboxamide as a yellow solid (7.3 mg, 32.9%). LCMS (ESI) m/z 361.1, [M+H]⁺.

### Example 358: Synthesis of N-(8-(methylamino)-5-(oxazolo[4,5-b]pyridin-2-yl)-2,7-naphthyridin-3-yl)cyclopropanecarboxamide

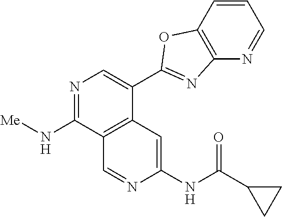 [SMILES: CNc1ncc(-c2nc3ncccc3o2)c2cc(NC(=O)C3CC3)ncc12]

### Step 1: 6-(cyclopropanecarboxamido)-N-(3-hydroxypyridin-2-yl)-1-(methylamino)-2,7-naphthyridine-4-carboxamide

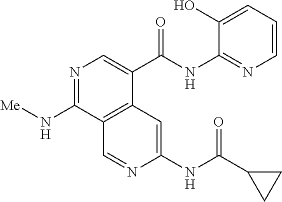 [SMILES: CNc1ncc(C(=O)Nc2ncccc2O)c2cc(NC(=O)C3CC3)ncc12]

To a stirred solution of 6-(cyclopropaneamido)-1-(methylamino)-2,7-naphthyridine-4-carboxylic acid (Example 356, step 2) (110.1 mg; 0.384 mmol, 1.00 eq.) and 2-aminopyridin-3-ol (51.2 mg; 0.463 mmol; 1.20 eq.) in DMA (2 mL) were added EDCI (110.2 mg; 0.574 mmol; 1.50 eq.) and DMAP (70.1 mg; 0.573 mmol; 1.50 eq.). The resulting mixture was stirred at room temperature for 2 hours under nitrogen atmosphere. The desired product was detected via LCMS. The mixture was purified by flash chromatography on pre-packed C18 column using 30-50% of MeCN in water (10 mmol/L NH₄HCO₃) as eluent to provide 6-(cyclopropanecarboxamido)-N-(3-hydroxypyridin-2-yl)-1-(methylamino)-2,7-naphthyridine-4-carboxamide as a white solid (40.3 mg, 27.5%). LCMS (ESI) m/z 379.1, [M+H]⁺.

### Step 2: N-(8-(methylamino)-5-(oxazolo[4,5-b]pyridin-2-yl)-2,7-naphthyridin-3-yl)cyclopropanecarboxamide

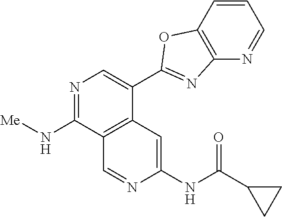 [SMILES: CNc1ncc(-c2nc3ncccc3o2)c2cc(NC(=O)C3CC3)ncc12]

A solution of 6-(cyclopropanecarboxamido)-N-(3-hydroxypyridin-2-yl)-1-(methylamino)-2,7-naphthyridine-4-carboxamide (40.3 mg; 0.106 mmol; 1.00 eq.) and P₂O₅(88.2 mg; 0.620 mmol; 6.00 eq.) in dry NMP (1 mL) was stirred at 140° C. overnight under nitrogen atmosphere. The desired product was detected via LCMS. The mixture was purified by flash chromatography on pre-packed C18 column using 30-50% of MeCN in water (10 mmol/L NH₄HCO₃) as eluent to provide N-(8-(methylamino)-5-(oxazolo[4,5-b]pyridin-2-yl)-2,7-naphthyridin-3-yl)cyclopropanecarboxamide as a brown solid (3.7 mg, 10.2%). LCMS (ESI) m/z 361.1, [M+H]⁺. ¹H NMR (400 MHz, DMSO-d₆) δ 11.09 (s, 1H), 9.75 (s, 1H), 9.46 (s, 1H), 9.00 (s, 1H), 8.83-8.78 (m, 1H), 8.49 (d, J=4.8 Hz, 1H), 8.16 (d, J=8.0 Hz, 1H), 7.40 (dd, J=8.0, 4.8 Hz, 1H), 3.11 (d, J=4.4 Hz, 3H), 2.15-2.07 (m, 1H), 0.96-0.82 (m, 4H).

### Example 359: Synthesis of N-(8-(methylamino)-5-(5-(piperidin-4-yl)benzo[d]oxazol-2-yl)-2,7-naphthyridin-3-yl)cyclopropanecarboxamide 2,2,2-trifluoroacetate

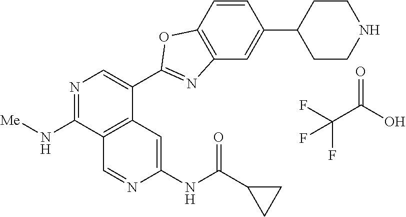

### Step 1: tert-butyl 4-(4-hydroxy-3-nitrophenyl)-3,6-dihydropyridine-1(2H)-carboxylate

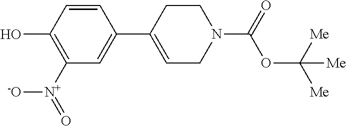

A solution of 4-bromo-2-nitrophenol (1.00 g; 4.588 mmol; 1.00 eq.) and tert-butyl 4-(4,4,5,5-tetramethyl-1,3,2-dioxaborolan-2-yl)-3,6-dihydropyridine-1(2H)-carboxylate (2.84 g; 9.184 mmol; 2.00 eq.) in a mixture solvent of 1,2-dimethoxyethane/water (4:1, 20 mL). To the above solution were added Pd(PPh₃)₄ (530.2 mg; 0.458 mmol; 0.10 eq.) and K₂CO₃ (633.9 mg; 4.587 mmol; 2.00 equiv) under nitrogen atmosphere. The resulting solution was stirred at 100° C. for 2 hours. The desired product was detected via LCMS. The reaction was concentrated under vacuum. The residue was purified by flash chromatography on silica gel column using 30-70% of EtOAc in petroleum ether as eluent to provide tert-butyl 4-(4-hydroxy-3-nitrophenyl)-3,6-dihydropyridine-1(2H)-carboxylate as a yellow solid (1.4 g, 95.9%). LCMS (ESI) m/z 321.1, [M+H]⁺.

### Step 2: tert-butyl 4-(3-amino-4-hydroxyphenyl)piperidine-1-carboxylate

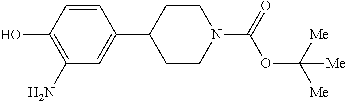 [SMILES: CC(C)(C)OC(=O)N1CCC(c2ccc(O)c(N)c2)CC1]

A solution of tort-butyl 4-(4-hydroxy-3-nitrophenyl)-3,6-dihydropyridine-1(2H)-carboxylate (900.0 mg; 5.310 mmol, 1.00 eq.) in MeOH (20 mL) was added 10% Pd/C (450.0 mg; 10% w/w) under nitrogen atmosphere. The mixture was hydrogenated at room temperature for 2 hours under hydrogen atmosphere (2 atm). LC-MS indicated that the starting material was consumed. After filtration, the filtrate was concentrated under reduced pressure to afford tert-butyl 4-(3-amino-4 hydroxyphenyl)piperidine-1-carboxylate as a yellow solid (800.0 mg, crude). The crude product was used in the next step directly without further purification. LCMS (ESI) m/z 293.1, [M+H]⁺.

### Step 3: tert-butyl 4-(2-(6-(cyclopropanecarboxamido)-4-(methylamino)-2,7-naphthyridin-4-yl)benzo[d]oxazol-5-yl)piperidine-1-carboxylate

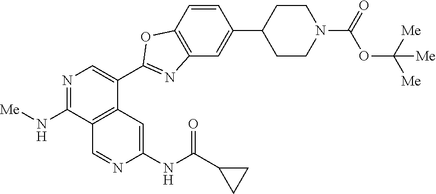 [SMILES: CNc1ncc(-c2nc3cc(C4CCN(C(=O)OC(C)(C)C)CC4)ccc3o2)c2cc(NC(=O)C3CC3)ncc12]

A solution of tell-butyl 4-(3-amino-4-hydroxyphenyl)-3,6-dihydropyridine-1(2H)-carboxylate (535.0 mg; 1.830 mmol; 1.00 eq.) and N-(5-formyl-8-(methylamino)-2,7-naphthyridin-3-yl)cyclopropanecarboxamide (494.6 mg; 1.830 mmol; 1.00 eq.) in toluene/DMSO (5:1, 12 mL) was stirred at 110° C. overnight under air atmosphere. The resulting mixture was concentrated under reduced pressure. The residue was dissolved in CH₂Cl₂ (10 mL). To the above mixture was added DDQ (456.9 mg; 2.013 mmol; 1.10 eq.). The resulting mixture was stirred at room temperature for 2 hours. The resulting mixture was concentrated under reduced pressure. The residue was purified by flash chromatography on pre-packed C18 column using 20-50% of MeCN in water (10 mmol/L NH₄HCO₃) as eluent to provide tert-butyl 4-(2-(6-(cyclopropanecarboxamido)-1-(methylamino)-2,7-naphthyridin-4-yl)benzo[d]oxazol-5-yl)piperidine-1-carboxylate as a yellow solid (200.0 mg, 19.14%). LCMS (ESI) m/z 543.2, [M+H]⁺.

### Step 4: N-(8-(methylamino)-5-(5-(piperidin-4-yl)benzo[d]oxazol-2-yl)-2,7-naphthyridin-3-yl)cyclopropanecarboxamide 2,2,2-trifluoroacetate

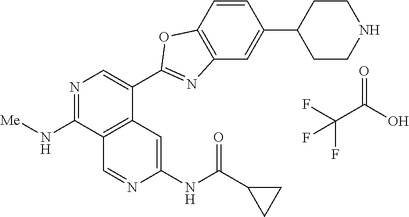

To a solution of tert-butyl 4-(2-(6-(cyclopropanecarboxamido)-1-(methyl amino)-2,7-naphthyridin-4-yl)benzo[d]oxazol-5-yl)piperidine-1-carboxylate (100.0 g; 0.184 mmol; 1.00 eq.) in MeOH (10 mL) was added a solution of HCl in dioxane (4N, 2 mL). The mixture was stirred at room temperature for 1 hour. The desired product was detected via. LCMS. The reaction was concentrated under vacuum. The residue was purified by flash chromatography on pre-packed C18 column using 25-80% of MeOH in water (0.1% 2,2,2-trifluoroacetic acid) to provide N-(8-(methyl amino)-5-(5-(piperidin-4-yl)benzo[d]oxazol-2-yl)-2,7-naphthyridin-3-yl)cyclopropanecarboxamide 2,2,2-trifluoroacetate as a yellow solid (20.7 mg, 25.0%). LCMS (ESI) m/z 443.2, [M+H]⁺. ¹H, NMR (400 MHZ, DMSO-d₆) δ 11.13 (s, 1H), 9.74 (s, 1H), 9.48 (s, 1H), 9.01-8.94 (m, 1H), 8.85 (s, 1H), 8.70-8.63 (m, 1H), 8.41-8.38 (m, 1H), 7.71 (d, J=8.4 Hz, 1H), 7.60 (d, J=2.4 Hz, 1H), 7.27 (dd, J=8.4, 2.4 Hz, 1H), 3.50-3.41 (m, 2H), 3.19-2.97 (m, 6H), 2.19-2.09 (m, 1H), 2.09-1.99 (m, 2H), 1.99-1.71 (m, 2H), 0.99-0.80 (m, 4H).

### Example 360: Synthesis of N-(8-(methylamino)-5-(5-(1-(oxetan-3-yl)piperidin-4-yl)benzo[d]oxazol-2-yl)-2,7-naphthyridin-3-yl) cyclopropanecarboxamide

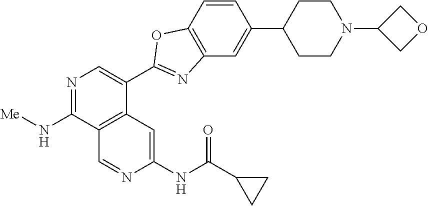 [SMILES: CNc1ncc(-c2nc3cc(C4CCN(C5COC5)CC4)ccc3o2)c2cc(NC(=O)C3CC3)ncc12]

A mixture of N-(8-(methylamino)-5-(5-(piperidin-4-yl)benzo[d]oxazol-2-yl)-2,7-naphthyridin-3-yl)cyclopropanecarboxamide (Example 359) (100.0 mg; 0.226 mmol; 1.00 eq.), oxetan-3-one (33.1 mg; 0.459 mmol; 2.00 eq.) and DIEA (146.1 mg; 1.130 mmol; 5.00 eq.) in CH₂Cl₂ (5 mL) was stirred at room temperature for 2 hours. To the above mixture was added NaBH(OAc)₃ (95.8 mg; 0.452 mmol; 2.00 eq). The resulting solution was stirred at room temperature for 8 hours. The desired product was detected via LCMS. The resulting mixture was concentrated under reduced pressure. The residue was purified by flash chromatography on silica gel column using 0-15% of MeOH in CH₂Cl₂ (0.1% trimethylamine) as eluent to afford a crude product. The crude product was slurried in MeOH (3 mL) at room temperature for 3 hours. After filtration, the solid was collect to provide N-(8-(methylamino)-5-(5-(1-(oxetan-3-yl)piperidin-4-yl)benzo[d]oxazol-2-yl)-2,7-naphthyridin-3-yl)cyclopropanecarboxamide as a light yellow solid (17.3 mg, 15.0%), LCMS (ESI) m/z 499.2, [MH-H]⁺. ¹H NMR (400 MHz, DMSO-d₆) δ 11.03 (s, 1H), 9.68 (s, 1H), 9.44 (s, 111), 8.89 (s, 1H), 8.70-8.60 (m, 1H), 7.64-7.59 (m, 2H), 7.29-7.27 (m, 4.58-4.53 (m, 2H), 4.52-4.41 (m, 2H), 3.47-3.39 (m, 1H), 3.09 (d, J=4.4 Hz, 3H), 2.90-2.82 (m, 2H), 2.70-2.65 (m, 1H), 2.14-2.07 (m, 1H), 1.93-1.73 (m, 6H), 0.96-0.85 (m, 4H).

### Example 361: Synthesis of N-(8-(methylamino)-5-(5-(1-ethylpiperidin-d-yl)benzo[d]oxazol-2-yl)-2,7-naphthyridin-3-yl)cyclopropanecarboxamide

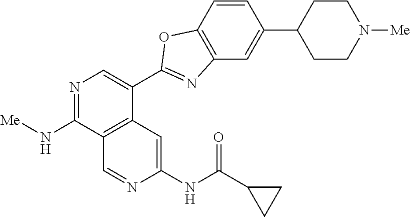 [SMILES: CNc1ncc(-c2nc3cc(C4CCN(C)CC4)ccc3o2)c2cc(NC(=O)C3CC3)ncc12]

A mixture of N-(8-(methylamino)-5-(5-(piperidin-4-yl)benzo[d]oxazol-2-yl)-2,7-naphthyridin-3-yl)cyclopropanecarboxamide 2,2,2-trifluoroacetate (Example 359) (90.0 mg; 0.203 mmol; 1.00 eq.), HCHO (37% in water, 0.1 mL) and DIEA (262.9 mg; 2.034 mmol; 10.00 eq.) in CH₂Cl₂ (10 mL) was stirred at room temperature for 2 hours. To the above mixture was added NaBH(OAc)₃ (129.3 mg; 0.610 mmol; 3.00 eq.), the resulting solution was stirred at room temperature overnight. The desired product was detected via LCMS. The resulting mixture was concentrated under reduced pressure. The residue was purified by flash chromatography on silica gel column using 0-15% of MeOH in CH₂Cl₂ (0.1% trimethylamine) as eluent to provide a crude product. The crude product was purified by flash chromatography on pre-packed C18 column using 20-80% of MeOH in water (10 mmol/L NH₄HCO₃) as eluent to provide N-(8-(methylamino)-5-(5-(1-methylpiperidin-4-yl)benzo[d]oxazol-2-yl)-2,7-naphthyridin-3-yl)cyclopropanecarboxamide as a yellow solid (12.1 mg, 12.6%). LCMS (ESI) m/z 457.2, [M+H]⁺. ¹H NMR (400 MHz, DMSO-d₆) 11.05 (s, 1H), 9.69 (s, 1H), 9.44 (s, 1H), 8.90 (s, 1H), 8.71-8.62 (m, 1H), 7.67 (d, J=8.0 Hz, 1H), 7.60 (d, J=2.0 Hz, 1H), 7.32-7.26 (in H), 3.30-3.17 (m, 2H), 3.09 (d, J=4.4 Hz, 3H), 2.89-2.82 (m, 1H), 2.78-2.70 (m, 1H), 2.66-2.56 (m, 4H), 2.16-2.07 (m, 1H), 2.04-1.81 (m, 4H), 0.93-0.86 (m, 4H).

### Example 362: Synthesis of N-(5-(5-methoxybenzo[d]oxazol-2-yl)-8-((methyl-d₃)amino)-2,7-naphthyridin-3-yl)cyclopropanecarboxamide

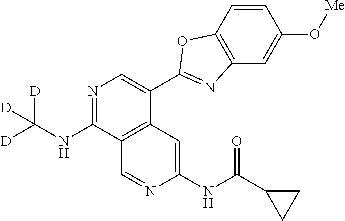 [SMILES: [2H]C([2H])([2H])Nc1ncc(-c2nc3cc(OC)ccc3o2)c2cc(NC(=O)C3CC3)ncc12]

### Step 1: 6-chloro-N-(methyl-d₃)-2,7-naphthyridin-1-amine

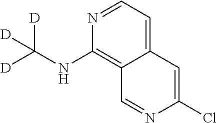 [SMILES: [2H]C([2H])([2H])Nc1nccc2cc(Cl)ncc12]

To a solution of 6-chloro-2,7-naphthyridin-1(2H)-one (10.00 g; 55.370 mmol; 1.00 eq.) and PyBOP (60.00 g; 115.290 mmol; 2.08 eq.) in DMA (200 mL) was added DIEA (60.05 g; 464.220 mmol; 8.38 eq.) at room temperature. The mixture was stirred at 40° C. for 3 hours under nitrogen atmosphere. To the above solution was added methan-d₃-amine hydrochloride (10.1 g; 41.750 mmol; 2.56 eq.). The resulting mixture was stirred at 40° C. for 16 hours. The desired product was detected via LCMS. The resulting mixture was diluted with brine (600 mL) and extracted with EtOAc (3×200 mL). The combined organic lavers were washed with brine (3×100 mL) and dried over anhydrous-Na₂SO₄, After filtration, the filtrate was concentrated under reduced pressure. The residue was purified by flash chromatography on silica gel column using 2-6% of MeOH in CH₂Cl₂ as eluent to afford 6-chloro-N-(methyl-d₃)-2,7-naphthyridin-1-amine as an off-white solid (10.0 g, 91.8%). LCMS (ESI) m/z 197.1, [M+H]⁺.

### Step 2: N-(8-((methyl-d₃)amino)-2,7-naphthyridin-3-yl)cyclopropanecarboxamide

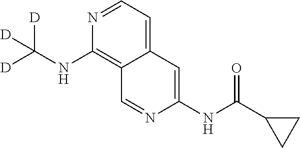 [SMILES: [2H]C([2H])([2H])Nc1nccc2cc(NC(=O)C3CC3)ncc12]

To a solution of 6-chloro-N-(methyl-d₃)-2,7-naphthyridin-1-amine (10.0 g; 50.850 mmol; 1.00 eq.) and cyclopropanecarboxamide (6.50 g; 76.370 mmol; 1.50 eq.) in 1,4-dioxane (100 mL) were added Cs₂CO₃ (7.8 g; 102.45 mmol; 2.00 eq.), Pd₂(dba)-3 (5.00 g; 5.460 mmol; 0.11 eq.) and XantPhos (600 g; 10.360 mmol; 0.20 eq.) at room temperature. The resulting mixture was stirred at 110° C. for 16 hours under nitrogen atmosphere. The desired product was detected via LCMS. The mixture was allowed to cool down to room temperature. The resulting mixture was diluted with EtOAc (500 mL). The resulting mixture was filtered, the filtrate was concentrated under reduced pressure. The residue was purified by flash chromatography on silica gel column using 3-10% of MeOH, in CH₂Cl₂ as eluent to afford N-(8-((methyl-d₃)amino)-2,7-naphthyridin-3-yl)cyclopropanecarboxamide as a white solid (11.12 g, 88.1%), LCMS (ESI) m/z 246.1, [M+H]⁺.

### Step 3: N-(5-bromo-8-((methyl-d₃)amino)-2,7-naphthyridin-3-yl)cyclopropanecarboxamide

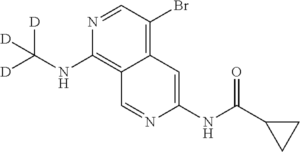 [SMILES: [2H]C([2H])([2H])Nc1ncc(Br)c2cc(NC(=O)C3CC3)ncc12]

A solution of N-(8-((methyl-d₃)amino)-2,7-naphthyridin-3-yl)cyclopropanecarboxamide (9.00 g; 36.690 mmol; 1.00 eq.) and NBS (5.22 g; 29.350 mmol; 0.80 eq.) in DMF (30 mL) was stirred at room temperature for 1 hour. The desired product was detected via LCMS. The resulting mixture was diluted with water (300 mL). The precipitated solids were collected by filtration and washed with water (100 mL). The solids were dried under vacuum to afford N-(5-bromo-8-((methyl-d₃)amino)-2,7-naphthyridin-3-yl)cyclopropanecarboxamide as a white solid (9.50 g, 79.8%). LCMS (ESI) m/z 324.0, [M+H]⁺.

### Step 4: N-(5-(hydroxymethyl)-8-((methyl-d₃)amino)-2,7-naphthyridin-3-yl)cyclopropanecarboxamide

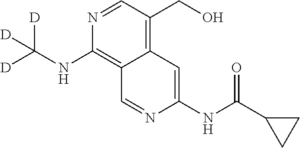 [SMILES: [2H]C([2H])([2H])Nc1ncc(CO)c2cc(NC(=O)C3CC3)ncc12]

A solution of (tributylstannyl)methanol (3.00 g; 9.343 mmol; 3.03 eq.), N-(5-bromo-8-((methyl-d₃)amino)-2,7-naphthyridin-3-yl)cyclopropanecarboxamide (1.00 g; 3.085 mmol; 1.00 eq.) and Pd(PPh₃)₄ (O 0.80 g; 0.692 mmol; 0.22 eq.) in 1,4-dioxane (50 mL) was stirred at 100° C. for 12 hours under nitrogen atmosphere. The mixture was concentrated under vacuum. The residue was purified by flash chromatography on silica gel column using 0-25% of MeOH in CH₂Cl₂ as eluent to provide N-(5-(hydroxymethyl)-8-((methyl-d₃)amino)-2,7-naphthyridin-3-yl)cyclopropanecarboxamide as a brown solid (500.0 mg, 58.9%). LCMS (ESI) m/z 276.1, [M+H]⁺.

### Step 5: N-(5-formyl-8-((methyl-d₃ amino)-2,7-naphthyridin-3-yl)cyclopropanecarboxamide

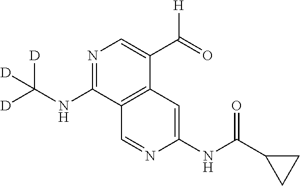 [SMILES: [2H]C([2H])([2H])Nc1ncc(C=O)c2cc(NC(=O)C3CC3)ncc12]

A solution of N-(5-(hydroxymethyl)-8-((methyl-d₃)amino)-2,7-naphthyridin-3-yl)cyclopropanecarboxamide (500.0 mg; 1.816 mmol; 1.00 eq.) in DMSO (10 mL) was heated until all of solids were dissolved. To this mixture were added CH₂Cl₂ (50 mL), pyridine (300.0 mg; 3.793 mmol; 2.09 eq.) and Dess-Martin (800.0 mg; 1.886 mmol; 1.04 eq.). The resulting solution was stirred at room temperature for 2 hours. The desired product was detected via LCMS. The mixture was filtered, and the filter cake was washed by CH₂Cl₂/MeOH. The filtrate was collected and concentrated under reduced pressure. The resulting mixture was diluted with EtOAc (300 mL) and washed by brine (3×50 mL). The organic layer was dried over anhydrous sodium sulfate and concentrated under vacuum until a large amount of solid precipitated. The precipitated product was collected by filtration to afford the crude product. This crude product was slurred in MeOH for 4 hours to afford N-(5-formyl-8-((methyl-d₃)amino)-2,7-naphthyridin-3-yl)cyclopropanecarboxamide as an offs-white solid (250.0 mg, 50.3%), LCMS (ESI) m/z 274.1, [M+H]⁺.

### Step 6: N-(5-(5-methoxybenzo[d]oxazol-2-yl)-8-((methyl-yl)cyclopropanecarboxamide

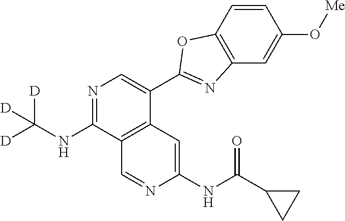 [SMILES: [2H]C([2H])([2H])Nc1ncc(-c2nc3cc(OC)ccc3o2)c2cc(NC(=O)C3CC3)ncc12]

N-(5-(5-methoxybenzo[d]oxazol-2-yl)-8-((methyl-d₃)amino)-2,7-naphthyridin-3-yl)cyclopropanecarboxamide was synthesized using a similar procedure that was previously, described in Example 210, by using N-(5-formyl-8-((methyl-d₃)amino)-2,7-naphthyridin-3-yl)cyclopropanecarboxamide and 2-amino-4-methoxyphenol as the starting material. LCMS (ESI) m/z 393.2, [M+H]⁺. ¹H NMR (400 MHz, DMSO-d₆) δ 11.03 (s, 1H), 9.64 (s, 1H), 9.44 (s, 1H), 8.89 (s, 1H), 8.65-8.58 (m, 1H), 7.62 (d, J=8.8 Hz, 1H), 7.28 (d, J=2.4 Hz, 1H), 7.00-6.92 (m, 1H), 3.86 (s, 3H), 2.15-2.06 (m, 1H), 0.94-0.83 (m, 4H).

### Example 363: Synthesis of N-(5-(4-methoxybenzo[d]oxazol-2-yl)-8-((methyl-d₃)amino)-2,7-naphthyridin-3-yl)cyclopropanecarboxamide

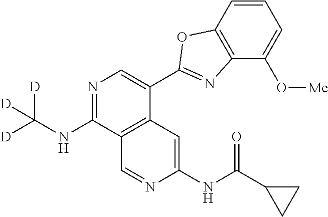 [SMILES: [2H]C([2H])([2H])Nc1ncc(-c2nc3c(OC)cccc3o2)c2cc(NC(=O)C3CC3)ncc12]

### Step 1: N-(8-((methyl-d₃)amino)-5-(4,4,5,5-tetramethyl-1,3,2-dioxaborolan-2-yl)-2,7-naphthyridin-3-yl)cyclopropanecarboxamide

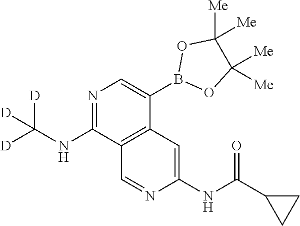 [SMILES: [2H]C([2H])([2H])Nc1ncc(B2OC(C)(C)C(C)(C)O2)c2cc(NC(=O)C3CC3)ncc12]

A mixture of N-(5-bromo-8-((methyl-d₃)amino)-2,7-naphthyridin-3-yl)cyclopropanecarboxamide (1.50 g; 4.627 mmol; 1.00 eq.), 4,4,4′,4′,5,5,5′,5′-octamethyl-2,2′-bi(1,3,2-dioxaborolane) (11.80 g; 46.468 mmol; 10.04 eq.), Pd(dppf)Cl₂. CH₂Cl₂ (3790.0 mg; 0.465 mmol; 0.10 eq.) and KOAc (910.0 mg; 9.272 mmol, 2.00 eq.) in 1,4-dioxane (75 mL) was stirred at 100° C. for 2 hours under nitrogen atmosphere. The desired product was detected via LCMS. The resulting mixture was concentrated under reduced pressure. The residue was dissolved in petroleum ether/EtOAc (10:1, 50 mL), whereupon the precipitated product was isolated via filtration and washed with petroleum ether/EtOAc(10:1, 20 mL). The solids were dried under reduced pressure to afford N-(8-((methyl-d₃)amino)-5-(4,4,5,5-tetramethyl-1,3,2-dioxaborol an-2-yl)-2,7-naphthyridin-3-yl)cyclopropanecarboxamide as a brown crude solid (2.6 g, crude). LCMS (ESI) m/z 372.2, [M+H]⁺.

### Step 2: N-(5-(4-methoxybenzo(dioxazol-2-yl)-8-((methyl-d₃)amino)-2,7-naphthyridin-3-yl) cyclopropanecarboxamide

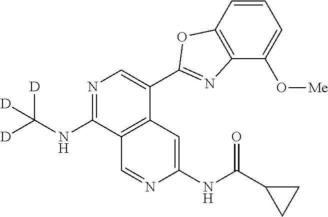 [SMILES: [2H]C([2H])([2H])Nc1ncc(-c2nc3c(OC)cccc3o2)c2cc(NC(=O)C3CC3)ncc12]

A suspension of N-(8-((methyl-d₃)amino)-5-(4,4,5,5-tetramethyl-1,3,2-dioxaborolan-2-yl)-2,7-naphthyridin-3-yl)cyclopropanecarboxamide (276.0 mg; 0.743 mmol; 1.00 eq.), 2-chloro-4-methoxybenzo[d]oxazole (135.0 mg; 0.735 mmol; 0.99 eq.), XPhos Pd G₃ (62.9 mg; 0.074 mmol; 0.10 eq.), XPhos (35.4 mg; 0.074 mmol; 0.10 eq.) and K₃PO₄ (473.0 mg; 2.228 mmol; 3.00 eq.) in 1,4-dioxane/water (5:1, 7.2 mL) was stirred at 90° C. for 1 hour under nitrogen atmosphere. The desired product was detected via LCMS. The resulting mixture was cooled to room temperature and concentrated under reduced pressure. The residue was purified by flash chromatography on silica gel column using 0-10% of MeOH in (HCl as eluent to provide the crude product. The crude product was purified by C18 column using 30-50% of MeCN in water (10 mmol/L NH₄HCO₃) as eluent to provide N-(5-(4-methoxybenzo[d]oxazol-2-yl)-8-((methyl-d₃)amino)-2,7-naphthyridin-3-yl)cyclopropanecarboxamide as a light yellow solid (54.7 mg, 18.7%). LCMS (ESI) m/z 393.2, [M+H]⁺. ¹H NMR (400 MHz, DMSO-d₆)d 11.03 (s, 9.64 (s, 1H), 9.43 (s, 1H), 8.86 (s, 1H), 8.62-8.58 (m, 1H), 7.33-7.24 (m, 2H), 6.94-6.89 (m, 1H), 4.17 (s, 3H), 2.13-2.05 (m, 1H), 0.90-0.82 (m, 4H).

### Example 364: Synthesis of N-(8-((methyl-d₃)amino)-5-(5-morpholinobenzo[d]oxazol-2-yl)-2,7-naphthyridin-3-yl)cyclopropanecarboxamide

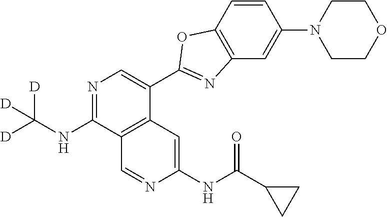 [SMILES: [2H]C([2H])([2H])Nc1ncc(-c2nc3cc(N4CCOCC4)ccc3o2)c2cc(NC(=O)C3CC3)ncc12]

A solution of 5-morpholinobenzo[d]oxazole (Example 345, step 3) (39.3 mg; 0.192 mmol; 1.20 eq.) and N-(5-bromo-8-((methyl-di)amino)-2,7-naphthyridin-3-yl)cyclopropanecarboxamide (52.0 mg; 0.160 mmol; 1.00 eq.) in DMF (2 mL). To the above solution were added Pd(PPh₃)₄ (18.5 mg; 0.016 mmol; 0.10 eq.) and Cs₂CO₃ (156.8 mg; 0.481 mmol; 3.00 eq.) under nitrogen atmosphere. The resulting solution was stirred at 110° C. overnight. The desired product was detected via. LCMS. The mixture was concentrated under reduced pressure. The residue was purified by flash chromatography on silica gel column using 5-25% of MeOH in CH₂Cl₂ as eluent to provide N-(8-((methyl-d₃)amino)-5-(5-morpholinobenzo[d]oxazol-2-yl)-2,7-naphthyridin-3-yl)cyclopropanecarboxamide as a yellow solid (51.8 mg, 69.1%). LC MS (ESI) m/z 448.2, [M+H]⁺. ¹H NMR (400 MHz, DMSO-d₆) δ 11.02 (s, 1H), 9.65 (s, 1H), 9.44 (s, 1H), 8.88 (s, 1H), 8.69-8.46 (m, 1H), 7.58 (d, J=8.8 Hz, 1H), 7.23 (d, J=2.4 Hz, 1H), 7.10-6.91 (m, 1H), 3.88-3.70 (m, 4H), 3.22.-3.10 (m, 4H), 2.18-2.02 (m, 1H), 0.98-0.80 (m, 4H).

### Example 365: Synthesis of N-(5-(5-(methoxy-d₃)benzo[d]oxazol-2-yl)-8-((methyl-d₃)amino)-2,7-naphthyridin-3-yl)cyclopropanecarboxamide

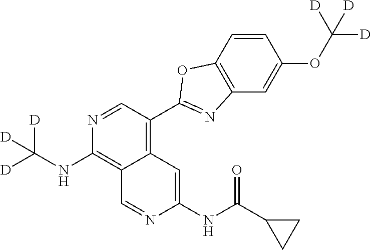 [SMILES: [2H]C([2H])([2H])Nc1ncc(-c2nc3cc(OC([2H])([2H])[2H])ccc3o2)c2cc(NC(=O)C3CC3)ncc12]

### Step 1: 5-(methoxy-d₃)benzo[d]oxazole

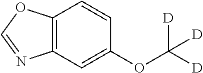 [SMILES: [2H]C([2H])([2H])Oc1ccc2ocnc2c1]

A solution of benzo[d]oxazol-5-ol (250.0 mg; 1.850 mmol, 1.00 eq.), iodomethane-d; (804.6 mg; 5,551 mmol; 3.00 eq.) and Cs₂CO₃ (1808.4 mg; 5.550 mmol; 3.00 eq.) in DMF (5 mL) was stirred at 60° C. overnight under nitrogen atmosphere. The desired product was detected via LCMS. The solution was diluted with water (10 mL), extracted with EtOAc (3×50 mL). The combined organic layers were washed with brine (3×10 mL), dried over anhydrous Na₂SO₄. After filtration, the filtrate was concentrated under reduced pressure. The residue was purified by flash chromatography on silica gel column using 10-30% of EtOAc in petroleum ether as eluent to provide 5-(methoxy-d₃)benzo[d]oxazole as a pink solid (240.0 mg, 81.0 ?/O ). LCMS (ESI) m/z 153.1, [M+H]⁺.

### Step 2: N-(5-(5-(methoxy-d₃)benzo[d]oxazol-2-yl)-8-((methyl-d₃)amino)-2,7-naphthyridin-3-yl)cyclopropanecarboxamide

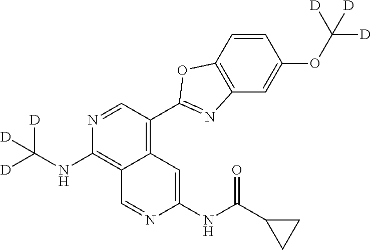 [SMILES: [2H]C([2H])([2H])Nc1ncc(-c2nc3cc(OC([2H])([2H])[2H])ccc3o2)c2cc(NC(=O)C3CC3)ncc12]

N-(5-(5-(methoxy-d₃)benzo[d]oxazol-2-yl)-8-((methyl-d₃)amino)-2,7-naphthyridin-3-yl)cyclopropanecarboxamide was synthesized using a similar procedure that was previously, described in Example 364 by using N-(5-bromo-8-((methyl-d₃)amino)-2,7-naphthyridin-3-yl)cyclopropanecarboxamide and 5-(methoxy-d₃)benzo[d]oxazole as the starting material. LCMS (ESI) m/z 396.2 [M+H]⁺. ¹H NMR (400 MHz, DMSO-d₆) δ 11.15 (s, 1H), 9.68 (s, 1H), 9.51 (s, 114), 9.30-8.96 (m, 1H), 8.78 (s, 1H), 7.65 (d, J=8.8 Hz, 1H), 7.30 (d, J=2.4 Hz, 1H), 6.05-6.94 (m, 1H), 2.17-2.08 (m, 0.95-0.85 (m, 4H).

### Example 366: Synthesis of (S)—N-(8-(ethylamino)-5-(5-(2-methylmorpholino)benzo[d]oxazol-2-yl)-2,7-naphthyridin-3-yl)cyclopropanecarboxamide

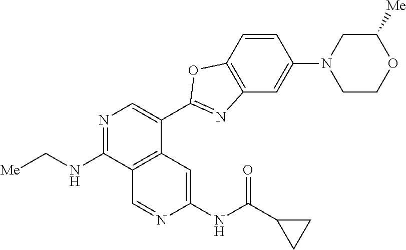 [SMILES: CCNc1ncc(-c2nc3cc(N4CCO[C@@H](C)C4)ccc3o2)c2cc(NC(=O)C3CC3)ncc12]

### Step 1: N-(8-(ethylamino)-5-vinyl-2,7-naphthyridin-3-yl)cyclopropanecarboxamide

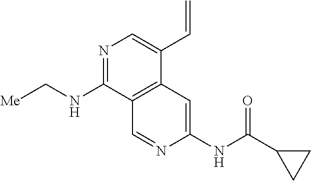 [SMILES: C=Cc1cnc(NCC)c2cnc(NC(=O)C3CC3)cc12]

To a solution of Pd(DtBPF)Cl₂ (0.40 g; 0.614 mmol; 0.21 eq.) and K₃PO₄ (1.60 g; 7.538 mmol; 2.53 eq.) in 1,4-dioxane/water (5.124 mL) were added N-(5-bromo-8-(ethylamino)-2,7-naphthyridin-3-yl)cyclopropanecarboxamide (Example 594, step 3) (1 g; 2.983 mmol; 1.00 eq.) and 4,4,5,5-tetramethyl-2-vinyl-1,3,2-dioxaborolane (0.80 g; 5,194 mmol; 1.74 eq.) under nitrogen atmosphere. The resulting solution was stirred at 90° C. for 2 hours. The reaction mixture was concentrated under vacuum. The residue was purified by flash chromatography on silica gel column using 2-10% of MeOH in CH₂Cl₂ to afford N-(8-(ethylamino)-5-vinyl-2,7-naphthyridin-3-yl)cyclopropanecarboxamide as a brown solid (800 mg, 94.9%). LCMS (ESI) m/z 283.1, [M+H]⁺.

### Step 2: N-(8-(ethylamino-5-formyl-2,7-naphthyridin-3-yl)cyclopropanecarboxamide

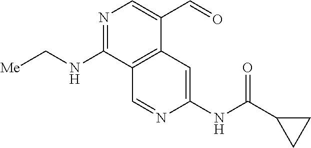 [SMILES: CCNc1ncc(C=O)c2cc(NC(=O)C3CC3)ncc12]

A solution of N-(8-(ethylamino)-5-vinyl-2,7-naphthyridin-3-yl)cyclopropanecarboxamide (350 mg; 1.240 mmol, 1.00 eq.) and 2,6-dimethylpyridine (266.0 mg; 2,482 mmol; 2.00 eq.) in a mixture solvent of 1,4-dioxane/water (1:1, 20 mL). To the above solution were added NaIO₄ (1.07 g; 5.003 mmol; 4.04 eq.) and K₂OsO₄·2H₂O (45.0 mg; 0.122 mmol; 0.10 eq.) at 0° C. under nitrogen atmosphere. The resulting solution was stirred at room temperature for 2 hours. The reaction was concentrated under vacuum. The residue was purified by flash chromatography on pre-packed C18 column using 30-60% of MeCN in water (10 mmol/L NH₄HCO₃) as eluent to provide N-(8-(ethylamino)-5-formyl-2,7-naphthyridin-3-yl)cyclopropanecarboxamide as a yellow solid (150 mg, 42.6%), LCMS (ESI) m/z 285.1, [M+H]⁺.

### Step 3: N-(5-(5-bromobenzo[d]oxazol-2-yl)-8-(ethylamino)-2,7-naphthyridin-3-yl)-cyclopropanecarboxamide

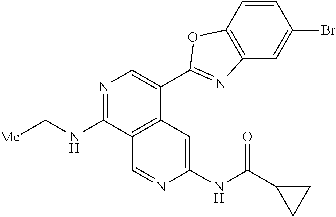 [SMILES: CCNc1ncc(-c2nc3cc(Br)ccc3o2)c2cc(NC(=O)C3CC3)ncc12]

A solution of N-(8-(ethylamino)-5-formyl-2,7-naphthyridin-3-yl)cyclopropanecarboxamide (100 mg; 0.352 mmol; 1.00 eq.) and 2-amino-4-bromophenol (70.0 mg; 0.372 mmol; 1.06 eq.) in toluene (3 mL) was stirred at 110° C. for 12 hours under nitrogen atmosphere. The reaction was concentrated under vacuum, the residue was dissolved in CH₂Cl₂ (5 mL). To the above solution was added DDQ (90.0 mg; 0.396 mmol; 1.13 eq.). The resulting mixture was stirred at room temperature for 2 hours. The reaction was concentrated under vacuum. The residue was purified by flash chromatography on pre-packed C18 column using 40-70% of MeCN in water (10 mmol/L: NH₄HCO₃) to provide N-(5-(5-bromobenzo[d]oxazol-2-yl)-8-(ethylamino)-2,7-naphthyridin-3-yl)cyclopropanecarboxamide as a brown solid (80 mg, 50.2%). LCMS (ESI) m/z 452.1, [M+H]⁺.

### Step 4: (S)—N-(8-(ethylamino)-5-(5-(2-methylmorpholin)benzo[d]oxazol-2-yl)-2,7-naphthyridin-3-yl)cyclopropanecarboxamide

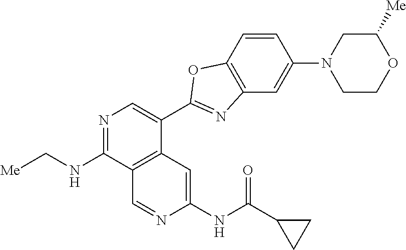 [SMILES: CCNc1ncc(-c2nc3cc(N4CCO[C@@H](C)C4)ccc3o2)c2cc(NC(=O)C3CC3)ncc12]

To a solution of EPhos Pd G₄ (35.0 mg; 0.038 mmol; 0.22 eq.), EPhos (30.0 mg; 0.038 mmol; 0.22 eq.) and Cs₂CO₃ (230.0 mg; 0.706 mmol; 3.99 eq.) in 1,4-dioxane (5 mL) were added N-(5-(5-bromobenzo[d]oxazol-2-yl)-8-(ethyl amino)-2,7-naphthyridin-3-yl)cyclopropanecarboxamide (80.0 mg; 0.177 mmol; 1.00 eq.) and (S)-2-methylmorpholine (80.0 mg; 0.791 mmol; 4.47 eq.) under nitrogen atmosphere. The resulting solution was stirred at 100° C. for 12 hours. The reaction was concentrated under vacuum. The residue was purified by flash chromatography on silica gel column using 2-10% of MeOH in CH₂Cl₂ to provide (S)-4-(8-(ethylamino)-5-(5-(2-methylmorpholino)benzo[d]oxazol-2-yl)-2,7-naphthyridin-3-yl)cyclopropanecarboxamide as a yellow solid (32.9 mg, 39.4%). LCMS (ESI) m/z 473.2, [M+H]⁺. ¹H NMR (400 MHz, DMSO-d₆) δ 11.04 (5, 1H), 9.64 (s, 1H), 9.48 (s, 1H), 8.86 (s, 1H), 8.71-8.64 (m, 1H), 7.57 (d, 8.8 Hz, 1H), 7.22 (d, J=2.4 Hz, 1H), 7.06 (dd, 1=8.8, 2.4 Hz, 1H), 3.98-3.90 (m, 1H), 3.75-3.58 (m, 5H), 3.57-3.49 (m, 1H), 2.78-2.67 (m, 1H), 2.45-2.37 (m, 1H), 2.14-2.09 (m, 1H), 1.28 (t, J=7.2 Hz, 3H), 1.19 (d, J=6.4 Hz, 3H), 0.97-0.81 (m, 4H).

### Example 367: Synthesis of N-(8-(cyclopropylamino)-5-(5-methoxybenzo[d]oxazol-2-yl)-2,7-naphthyridin-3-yl)cyclopropanecarboxamide

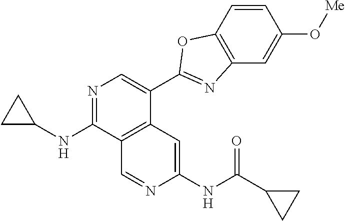 [SMILES: COc1ccc2oc(-c3cnc(NC4CC4)c4cnc(NC(=O)C5CC5)cc34)nc2c1]

To a solution of 5-methoxybenzo[d]oxazole (51.7 mg; 0.347 mmol; 1.20 eq.) and N-(5-bromo-8-(cyclopropylamino)-2,7-naphthyridin-3-yl)cyclopropanecarboxamide (100.0 mg; 0.288 mmol; 1.00 eq.) in DMF (3 mL) were added Pd(PPh₃)₄ (33.4 mg; 0.029 mmol; 0.10 eq.) and Cs₂CO₃ (282.7 mg; 0.868 mmol; 3.01 eq.) at room temperature. The reaction was stirred at 110° C. for 1 hour under nitrogen atmosphere. The desired product was detected via LCMS. The reaction was diluted with EtOAc (90 mL) and washed with saturated brine (3×10 mL). The organic phase was dried with Na₂SO₄, filtered and concentrated under reduced pressure. The residue was purified by flash chromatography on silica gel column using 1-9% of MeOH in CH₂Cl₂ as eluent to provide N-(8-(cyclopropylamino)-5-(5-methoxybenzo[d]oxazol-2-yl)-2,7-naphthyridin-3-yl)cyclopropanecarboxamide as a yellow solid (71.0 mg, 59.3%), LCMS (ESI) m/z 416.2, [M+H]⁺. 1H NMR (400 MHz, DMSO-d₆) δ 11.06 (s, 1H), 9.65 (s, 1H), 9.47 (s, 1H), 8.92 (s, 1H), 8.60 8.54 (m, 1H), 7.63 (d, J=8.8 Hz, 1H), 7.29 (d, J=2.4 Hz, 1H), 6.97 (dd, J=8.8, 2.4 Hz, 1H), 3.86 (s, 3H), 3.20-3.10 (m, 1H), 2.16-2.05 (m, 1H), 0.96-0.79 (m, 6H), 0.76-0.67 (m, 2H).

### Example 368: Synthesis of (S)—N-(8-amino-5-(5-(2-methylmorpholino)benzo[d]oxazol-2-yl)-2,7-naphthyridin-3-yl)cyclopropanecarboxamide

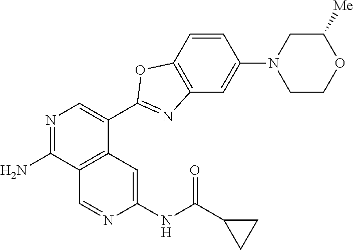 [SMILES: C[C@H]1CN(c2ccc3oc(-c4cnc(N)c5cnc(NC(=O)C6CC6)cc45)nc3c2)CCO1]

### Step 1: 6-chloro-N-(4-methoxybenzyl)-2,7-naphthyridin-1-amine

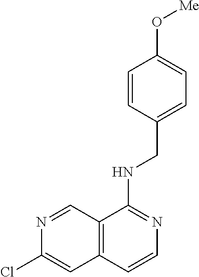 [SMILES: COc1ccc(CNc2nccc3cc(Cl)ncc23)cc1]

To a solution of 6-chloro-2H-2,7-naphthyridin-1-one (2.00 g; 11.075 mmol, 1.00 eq.), PyBOP (11.50 g; 22.098 mmol, 2.00 eq.) and DIEA (7.16 g; 55.375 mmol; 5.00 eq.) in DMA (30 mL) was added (4-methoxyphenyl)methanamine (3.04 g; 22.160 mmol; 2.00 eq.) under nitrogen atmosphere. The resulting solution was stirred at 90° C. overnight. The desired product was detected via LCMS. The resulting mixture was diluted with water (150 mL) and extracted with EtOAc (3×120 mL). The combined organic layers were concentrated under reduced pressure. To the mixture was added water (200 mL) to precipitate solids. The precipitated solids were collected by filtration and slurried in CH₂Cl₂ (200 mL). The solids were collected by filtration and washed with CH₂Cl₂ (2×20 mL) to afford 6-chloro-N-(4-methoxybenzyl)-2,7-naphthyridin-1-amine as off-white solid (4.2 g, crude). LCMS (ESI) m/z 301.1, [M+H]⁺.

### Step 2: N-(8-((4-methoxybenzyl)amino)-2,7-naphthyridin-3-yl)cyclopropanecarboxamide

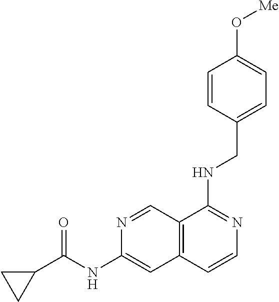 [SMILES: COc1ccc(CNc2nccc3cc(NC(=O)C4CC4)ncc23)cc1]

A solution of 6-chloro-N-(4-methoxybenzyl)-2,7-naphthyridin-1-amine (2.3 g; 7.673 mmol; 1.00 eq.), cyclopropanecarboxamide (0.99 g; 11.633 mmol; 1.52 eq.), Pd₂(dba)₃ (702.0 mg; 0.767 mmol; 0.10 equiv), XantPhos (888.0 mg; 1.535 mmol; 0.20 eq.) and Cs₂CO₃ (50.00 g; 15.346 mmol; 2.00 eq.) in 1,4-dioxane (50 mL) was stirred at 110° C. for 14 hours under nitrogen atmosphere. The desired product was detected via LCMS. The resulting mixture was filtered and concentrated under reduced pressure. The residue was purified by flash chromatography on silica gel column using 5-10% of MeOH in CH₂Cl₂ as eluent to provide N-(8-((4-methoxybenzyl)amino)-2,7-naphthyridin-3-yl)cyclopropanecarboxamide as an off-white solid (1 g, 65.5%). LCMS (ESI) m/z 349.2, [M+H]⁺.

### Step 3: N-(5-bromo-8-((4-methoxybenzyl)amino)-2,7-naphthyridin-3-yl)cyclopropanecarboxamide

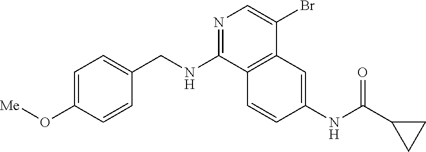 [SMILES: COc1ccc(CNc2ncc(Br)c3cc(NC(=O)C4CC4)ccc23)cc1]

To a solution of N-(8-((4-methoxybenzyl)amino)-2,7-naphthyridin-3-yl)cyclopropanecarboxamide (760.0 mg; 2.181 mmol; 1.00 eq.) in DMF (8 mL) was added NBS (388.0 mg; 2.180 mmol; 1.00 eq.) at 0° C. under nitrogen atmosphere. The resulting mixture was stirred at room temperature for 1 hour. The resulting mixture was diluted with EtOAc (150 mL) and washed with brine (5×30 mL). The organic layer was dried over anhydrous Na₂SO₄. After filtration, the filtrate was concentrated under reduced pressure. The residue was purified by flash chromatography on silica gel column using 5-10% of MeOH in CH₂Cl₂ as eluent to provide N-(5-bromo-8-((4-methoxybenzyl)amino)-2,7-naphthyridin-3-yl)cyclopropanecarboxamide as light yellow solid (580 mg, 62.3%). LCMS (ESI) m/z 427.1, [M+H]⁺.

### Step 4: N-(8-((4-methoxybenzyl)amino)-5-vinyl-2,7-naphthyridin-3-yl)cyclopropanecarboxamide

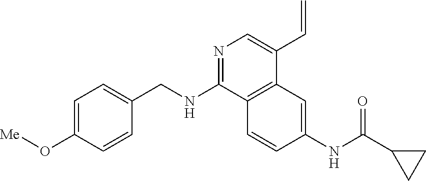 [SMILES: C=Cc1cnc(NCc2ccc(OC)cc2)c2ccc(NC(=O)C3CC3)cc12]

A solution of 4,4,5,5-tetramethyl-2-vinyl-1,3,2-dioxaborolane (206.0 mg; 1.337 mmol; 1.50 eq.) and N-(5-bromo-8-((4-methoxybenzyl)amino)-2,7-naphthyridin-3-yl)cyclopropanecarboxamide (380.0 mg; 0.889 mmol; 1.00 eq.) in a mixture solvent of 1,4-dioxane/water (5:1, 6 mL). To the above solution were added Pd(DtBPF)Cl₂ (58.0 mg; 0.089 mmol; 0.10 eq.) and K₃PO₄ (377.1 mg; 1.776 mmol; 2.00 eq.). The resulting mixture was stirred at 90° C. for 1 hour under nitrogen atmosphere. The desired product was detected via LCMS. The resulting mixture was concentrated under reduced pressure. The residue was purified by flash chromatography on silica gel column using 0-3% of MeOH in CH₂Cl₂ as eluent to provide N-(8-((4-methoxybenzyl)amino)-5-vinyl-2,7-naphthyridin-3-yl)cyclopropanecarboxamide as light yellow solid (200 mg, 60.8%). LCMS (ESI) m/z 375.2, [M+H]⁺.

### Step 5: N-(5-formyl-8-((4-methoxybenzyl)amino)-2,7-naphthyridin-3-yl)cyclopropanecarboxamide

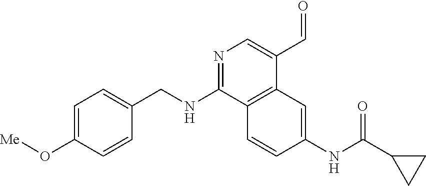 [SMILES: COc1ccc(CNc2ncc(C=O)c3cc(NC(=O)C4CC4)ccc23)cc1]

To a stirred solution of N-(8-((4-methoxybenzyl)amino)-5-vinyl-2,7-naphthyridin-3-yl)cyclopropanecarboxamide (200.0 mg; 0.534 mmol; 1.00 eq.) in a mixture solvent of 1,4-dioxane/water (1:1, 20 were added 2,6-dimethylpyridine (114.0 mg; 1,064 mmol; 1.99 eq.), NaIO₄ (457 mg; 2.137 mmol; 4.00 eq.) and K₂OsO₄·2H₂O (21.6 mg; 0.059 mmol; 0.11 eq.) in portions at 0° C. The resulting mixture was stirred at 0° C. for 2 hours. The desired product was detected via LCMS. The precipitated product was collected by filtration and washed with water (3×20 mL) to afford N-(5-formyl-8-((4-methoxybenzyl)amino)-2,7-naphthyridin-3-yl)cyclopropanecarboxamide as light yellow solid (650 mg, crude). LCMS (ESI) m/z 377.2, [M+H]⁺.

### Step 6: N-(5-(5-bromobenzo[d]oxazol-2-yl)-8((4-ethoxybenzyl)amino)-2,7-naphthyridin-3-yl)cyclopropanecarboxamide

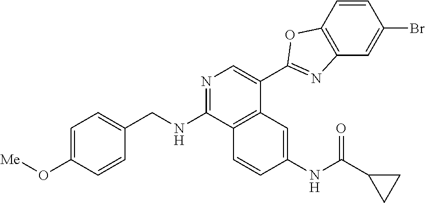 [SMILES: COc1ccc(CNc2ncc(-c3nc4cc(Br)ccc4o3)c3cc(NC(=O)C4CC4)ccc23)cc1]

A solution of N-(5-formyl-8-((4-methoxybenzyl)amino)-2,7-naphthyridin-3-yl)cyclopropanecarboxamide (380.0 mg; 1.010 mmol; 1.00 eq.) and 2-amino-4-bromophenol (189.8 mg; 1.009 mmol; 1.00 eq.) in toluene (10 mL) was stirred at 115° C. for 12 hours under nitrogen atmosphere. The mixture was concentrated under reduced pressure, the residue was dissolved in CH₂Cl₂ (10 mL). To the above mixture was added DDQ (252.1 mg; 1.11 mmol; 1.10 eq.) in portions at room temperature. The resulting mixture was stirred at room temperature for an additional 2 hours. The mixture was concentrated under reduced pressure. The residue was purified by flash chromatography on pre-packed C18 column using 40-60% of MeOH in water (10 mmol/L NH₄HCO₃) as eluent to provide N-(5-(5-bromobenzo[d]oxazol-2-yl)-8-((4-methoxybenzyl)amino)-2,7-naphthyridin-3-yl)cyclopropanecarboxamide as a off-white solid (158.0 mg, 28.8%). LCMS (ESI) m/z 544.1, [M+H]⁺.

### Step 7: (S)—N-(8-((4-methoxybenzyl)amino)-5-(5-(2-methylmorpholino)benzo[d]oxazol-2-yl)-2,7-naphthyridin-3-yl)cyclopropanecarboxamide

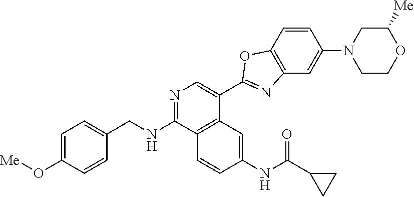 [SMILES: COc1ccc(CNc2ncc(-c3nc4cc(N5CCO[C@@H](C)C5)ccc4o3)c3cc(NC(=O)C4CC4)ccc23)cc1]

A solution of N-(5-(5-bromobenzo[d]oxazol-2-yl)-8-((4-methoxybenzyl)amino)-2,7-naphthyridin-3-yl)cyclopropanecarboxamide (70 mg; 0.129 mmol; 1 eq.), (S)-2-methylmorpholine (39.0 mg; 0.386 mmol; 3.00 eq.), EPhos Pd G₄ (23.6 mg; 0.026 mmol; 0.20 eq.), EPhos (13.7 mg; 0.026 mmol; 0.20 eq.) and Cs₂CO₃ (167.5 mg; 0.514 mmol; 4.00 eq.) in 1,4-dioxane (7 mL) was stirred at 110° C. for 14 hours under nitrogen atmosphere. The desired product was detected via LCMS. The mixture was allowed to cool down to room temperature. The resulting mixture was concentrated under reduced pressure. The residue was purified by flash chromatography on silica gel column using 3-6% of MeOH in CH₂Cl₂ as eluent to provide (S)—N-(8-((4-methoxybenzyl)amino)-5-(5-(2-methylmorpholino)benzo[d]oxazol-2-yl)-2,7-naphthyridin-3-yl)cyclopropanecarboxamide as an off-white solid (54.0 mg, 74.3%). LCMS (ESI) m/z 565.2, [M+H]⁺.

### Step 8: (SYN-(8-amino-5-(5-(2-methylmorpholino)benzo[d]oxazol-2-yl)-2,7-naphthyridin-3-yl)cyclopropanecarboxamide

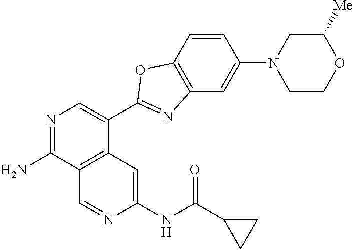 [SMILES: C[C@H]1CN(c2ccc3oc(-c4cnc(N)c5cnc(NC(=O)C6CC6)cc45)nc3c2)CCO1]

A solution of (S)—N-(8-((4-methoxybenzyl)amino)-5-(5-(2-methylmorpholino)benzo[d]oxazol-2-yl)-2,7-naphthyridin-3-yl)cyclopropanecarboxamide (70.0 mg; 0.124 mmol; 1.00 eq.) in TFA (4 mL) was stirred at 75° C. overnight. The desired product was detected via LCMS. The resulting mixture was concentrated under reduced pressure. The mixture was neutralized to pH=7 with saturated NaHCO₃ solution, extracted with EtOAc (4×15 mL). The combined organic layers were washed with brine (4×10 mL), dried over anhydrous Na₂SO₄. After filtration, the filtrate was concentrated under reduced pressure. The residue was purified by flash chromatography on silica gel column using 3-10% of MeOH in CH₂Cl₂ as eluent to provide (9-N-(8-amino-5-(5-(2-methylmorpholino)benzo[d]oxazol-2-yl)-2,7-naphthyridin-3-yl)cyclopropanecarboxamide as a yellow solid (31.6 mg, 57.3%), LCMS (ESI) m/z 445.2, [M+H]⁺. ¹H NMR (400 MHz, DMSO-d₆) δ 11.02 (s, 1H), 9.64 (s, 1H), 9.47 (s, 1H), 8.82 (s, 1H), 8.01 (s, 2H), 7.57 (d, J=8.8 Hz, 1H), 7.22 (d, J=2.4 Hz, 1H), 7.05 (dd, J=8.8, 2.4 Hz, 1H), 3.97-3.91 (m, 1H), 3.77-3.67 (m, 2H), 3.65-3.59 (m, 1H), 3.56-3.49 (m, 1H), 2.78-2.66 (m, 1H), 2.43-2.35 (m, 1H), 2.15-2.08 (m, 1H), 1.19 (d, J=6.4 Hz, 3H), 0.94-0.90 (m, 2H), 0.89-0.85 (m, 2H).

### Example 369: Synthesis of N-(8-amino-5-(5-methoxybenzo[d]oxazol-2-yl)-2,7-naphthyridin-3-yl)cyclopropanecarboxamide

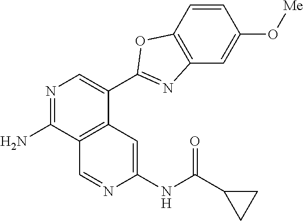 [SMILES: COc1ccc2oc(-c3cnc(N)c4cnc(NC(=O)C5CC5)cc34)nc2c1]

### Step 1: N-(5-(5-methoxybenzo[d]oxazol-2-yl)-8-((4-methoxybenzyl)amino)-2,7-naphthyridin-3-yl)cyclopropanecarboxamide

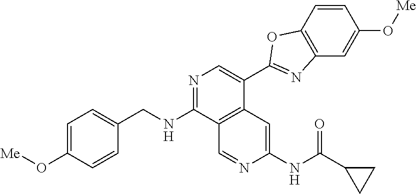 [SMILES: COc1ccc(CNc2ncc(-c3nc4cc(OC)ccc4o3)c3cc(NC(=O)C4CC4)ncc23)cc1]

To a stirred solution of N-(5-bromo-8-((4-methoxybenzyl)amino)-2,7-naphthyridin-3-yl)cyclopropanecarboxamide (Example 368, step 3) (100.0 mg; 0.234 mmol; 1.00 eq.) and 5-methoxybenzo[d]oxazole (41.9 mg; 0.281 mmol; 1.20 eq.) in DMF (5 mL) were added Pd(PPh₃)₄ (27.1 mg; 0.023 mmol; 0.10 eq.) and Cs₂CO₃ (228.7 mg; 0.702 mmol; 3.00 eq.). The resulting solution was stirred at 110 for 14 hours under nitrogen atmosphere. The desired product was detected via LCMS. The mixture was allowed to cool down to room temperature. The resulting mixture was diluted with water (10 mL) and extracted with EtOAc (3×10 mL). The combined organic layers were washed with brine (3×10 mL), dried over anhydrous Na₂SO₄. After filtration, the filtrate was concentrated under reduced pressure. The residue was purified by flash chromatography on silica gel column using 5-10% of MeOH in CH₂Cl₂ as eluent to provide N-(5-(5-methoxybenzo[d]oxazol-2-yl)-8-((4-methoxybenzyl)amino)-2,7-naphthyridin-3-yl)cyclopropanecarboxamide as a yellow solid (110.0 mg, 94.8%) LCMS (ESI) m/z 496.2, [M+H]⁺.

### Step 2: N-(8-amino-5-(5-methoxybenzo[d]oxazol-2-yl)-2,7-naphthyridin-3-yl)cyclopropanecarboxamide

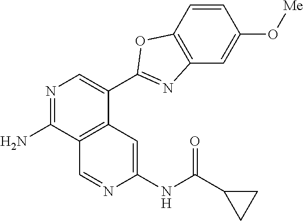 [SMILES: COc1ccc2oc(-c3cnc(N)c4cnc(NC(=O)C5CC5)cc34)nc2c1]

A solution of N-(5-(5-methoxybenzo[d]oxazol-2-yl)-8-((4-methoxybenzyl)amino)-2,7-naphthyridin-3-yl)cyclopropanecarboxamide (100.0 mg; 0.202 mmol; 1.00 eq.) in TFA (5 mL) was stirred at 75° C. overnight. The desired product could be detected by LCMS. The resulting mixture was diluted with toluene (10 mL) and concentrated under reduced pressure. The residue was purified by flash chromatography on silica gel column using 5-10% of MeOH in CH₂Cl₂ as eluent to provide N-(8-amino-5-(5-methoxybenzo[d]oxazol-2-yl)-2, 7-naphthyridin-3-yl)cyclopropanecarboxamide as a yellow solid (44.0 mg, 58.0%). LCMS (ESI) m/z 376.1, [M+H]⁺. ¹H NMR (400 MHz, DMSO-d₆) δ 11.03 (s, 1H), 9.64 (s, 1H), 9.47 (s, 1H), 8.83 (s, 1H), 8.03 (s, 2H), 7.62 (d, J=8.8 Hz, 1H), 7.28 (d, J=2.4 Hz, 1H), 6.95 (d, J=8.8, 2.4 Hz, 1H), 3.86 (s, 3H), 2.13-2.08 (m, 1H), 0.94-0.88 (m, 4H).

### Example 370: Synthesis of N-(8-((2,2-difluoroethyl)amino)-5-(5 oxybenzo[d]oxazol-2-yl)-2,7-naphthyridin-3-yl)cyclopropanecarboxamide

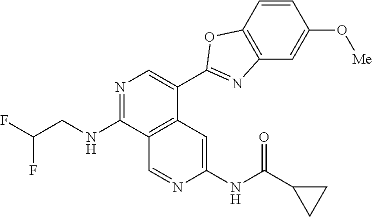 [SMILES: COc1ccc2oc(-c3cnc(NCC(F)F)c4cnc(NC(=O)C5CC5)cc34)nc2c1]

### Step 1: 1,6-dichloro-2,7-naphthyridine

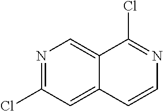 [SMILES: Clc1cc2ccnc(Cl)c2cn1]

A solution of 6-chloro-2,7-naphthyridin-1(2H)-one (500.1 mg; 2.760 mmol; 1.00 eq.) in phosphorus oxychloride (5 mL) was stirred at 110° C. for 2 hours under nitrogen atmosphere. The desired product was detected vis LCMS. The resulting mixture was concentrated under reduced pressure. The residue was dissolved with CH₂Cl₂ (50 mL), washed with saturated sodium bicarbonate (1×5 mL) and brine (2×5 mL). The organic layer was dried over anhydrous Na₂SO₄. After filtration, the filtrate was concentrated under reduced pressure to afford 1,6-dichloro-2,7-naphthyridine as a yellow solid (605.1 mg, crude). LCMS m/z 199.0, [M+H]⁺.

### Step 2: 6-chloro-N-(2,2-difluoroethyl)-2,7-naphthyridin-1-amine

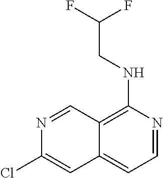 [SMILES: FC(F)CNc1nccc2cc(Cl)ncc12]

A solution of 1,6-dichloro-2,7-naphthyridine (605.1 mg; 3.040 mmol; 1.00 eq.), DIPEA (1.57 g; 12.160 mmol; 4.00 eq.) and 2,2-difluoroethan-1-amine (295.1 mg; 3.648 mmol; 1.20 eq.) in NMP (6 mL) was stirred at 100° C. for 5 hours under nitrogen atmosphere. The desired product was detected vis LCMS. The resulting mixture was diluted with EtOAc (50 mL), washed with brine (3×5 mL). The organic phase was dried over anhydrous Na₂SO₄. After filtration, the filtrate was concentrated under reduced pressure. The residue was purified by flash chromatography on silica gel column using 0-20% of EtOAc in petroleum ether as eluent to provide 6-chloro-N-(2,2-difluoroethyl)-2,7-naphthyridin-1-amine as a yellow solid (488.1 mg, 65.8%). LCMS (ESI) m/z 244.0, [M+H]⁺.

### Step 3: N-(8-((2,2-difluoroethyl amino)-2,7-naphthyridin-3-yl)cyclopropanecarboxamide

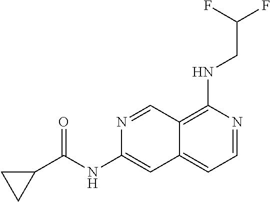 [SMILES: O=C(Nc1cc2ccnc(NCC(F)F)c2cn1)C1CC1]

A solution of 6-chloro-N-(2,2-difluoroethyl)-2,7-naphthyridin-1-amine (488.1 mg; 2.000 mmol; 1.00 eq.), cyclopropanecarboxamide (255.0 mg; 3.000 mmol, 1.50 eq.), Pd₂(dba)₃, (183.2 mg; 0.200 mmol; 0.10 eq.), XantPhos (231.3 mg; 0.401 mmol; 0.20 eq.) and Cs₂CO₃ (1.30 g; 4.00 mmol; 2.00 eq.) in 1,4-dioxane (5 mL) was stirred at 110° C. for 4 hours under nitrogen atmosphere. The desired product was detected via LCMS. The resulting mixture was concentrated under reduced pressure. The residue was purified by flash chromatography on silica gel column using 0-5% of MeOH in CH₂Cl₂ as eluent to provide N-(8-((2,2-difluoroethyl)amino)-2,7-naphthyridin-3-yl)cyclopropanecarboxamide as a yellow solid (430.2 mg, 73.4%). LCMS (ESI) m/z 293.1, [M+H]⁺.

### Step 4: N-(5-bromo-8-((2,2-difluoroethyl)amino)-2,7-naphthyridin-3-yl)cyclopropanecarboxamide

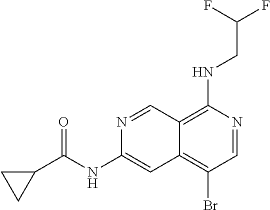 [SMILES: O=C(Nc1cc2c(Br)cnc(NCC(F)F)c2cn1)C1CC1]

To a stirred solution of N-(8-((2,2-difluoroethyl)amino)-2,7-naphthyridin-3-yl)cyclopropanecarboxamide (430.2 mg; 1.470 mmol; 1.00 eq.) in DMF (3 mL) was added NBS (261.1 mg; 1.470 mmol; 1.00 eq.) in portions at 0° C. under nitrogen atmosphere. The mixture was stirred at 0° C. for 1 hour. The desired product was detected vis LCMS. The reaction was quenched by the addition of water (15 mL) and extracted with EtOAc (3×20 mL). The combined organic layers were washed with brine (4×7 mL), dried over anhydrous Na₂SO₄, filtered, and concentrated under reduced pressure to provide N-(5-bromo-8-((2,2-difluoroethyl)amino)-2,7-naphthyridin-3-yl)cyclopropanecarboxamide as a yellow solid (500.2 mg, 91.5%). LCMS (ESI) m/z 371.0, [M+H]⁺.

### Step 5: N-(8-((2,2-difluoroethyl)amino)-5-(4,4,5,5-tetramethyl-1,3,2-dioxaborolan-2-yl)-2,7-naphthyridin-3-yl)cyclopropanecarboxamide

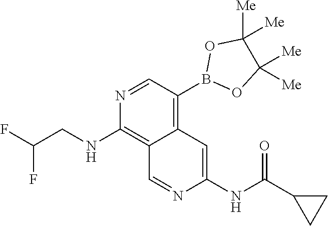 [SMILES: CC1(C)OB(c2cnc(NCC(F)F)c3cnc(NC(=O)C4CC4)cc23)OC1(C)C]

A solution of N-(5-bromo-8-((2,2-difluoroethyl)amino)-2,7-naphthyridin-3-yl)cyclopropanecarboxamide (250.1 mg; 0.674 mmol; 1.00 eq.), KOAc (132.2 mg; 1,348 mmol; 2.00 eq.), Pd(dppf)Cl₂CH₂Cl₂ (54.8 mg; 0.067 mmol; 0.10 eq.) and 4,4,4′,4′5,5,5′,5′-octamethyl-2,2′-bi(1,3,2-dioxaborolane) (1.71 g; 6.740 mmol, 10.00 eq.) in 1,4-dioxane (10 mL) was stirred at 100 for 3 hours under nitrogen atmosphere. The desired product was detected vis LCMS. The resulting mixture was concentrated under reduced pressure. The residue was dissolved in a petroleum ether/EtOAc (10:1, 10 mL), whereupon, the precipitated solids were collected by filtration and washed with a mixture solvent of petroleum ether/EtOAc(10:1, 5 mL). The solids were dried under reduced pressure to afford N-(8-((2,2-difluoroethyl)amino)-5-(4,4,5,5-tetramethyl-1,3,2-dioxaborolan-2-yl)-2,7-naphthyridin-3-yl)cyclopropanecarboxamide as a grey, solid (210.1 mg, crude). LCMS (ESI) m/z 419.2, [M+H]⁺.

### Step 6: N-(8-((2,2-difluoroethyl)amino)-5-(5-methoxybenzo[d]oxazol-2-yl)-2,7-naphthyridin-3-yl)cyclopropanecarboxamide

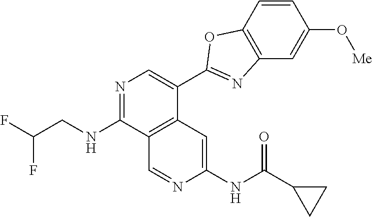 [SMILES: COc1ccc2oc(-c3cnc(NCC(F)F)c4cnc(NC(=O)C5CC5)cc34)nc2c1]

A solution of N-(8-((2,2-difluoroethyl)amino)-5-(4,4,5,5-tetramethyl-1,3,2-dioxaborolan-2-yl)-2,7-naphthyridin-3-yl)cyclopropanecarboxamide (210.1 mg; 0.502 mmol; 1.00 eq.), XPhos Pd (42.5 mg; 0.050 mmol; 0.10 eq.), XPhos (47.8 mg; 0.100 mmol; 0.20 eq.), K₃PO₄(213.1 mg; 1.004 mmol; 2.00 eq.) and 2-chloro-5-methoxybenzo[d]oxazole (92.1 mg; 0.502 mmol; 1.00 eq.) in 1,4-dioxane/water (5:1, 2.4 mL) was stirred at 90° C. for 1 hour under nitrogen atmosphere. The desired product was detected vis LCMS. The resulting mixture was concentrated under reduced pressure. The residue was purified by flash chromatography on silica gel column using 0-5% of MeOH in CH₂Cl₂ as eluent to provide the crude product. The crude product was purified by reverse phase preparative HPLC (Prep-C18, 5 μM XSelect CSH OBD column, 19×250 mm, waters; gradient elution of 80-85% MeOH in water over a 8 min period, where water contains 10 mmol/L NH₄HCO₃, flow rate: 20 mL/min, detector UV wavelength: 254 nm) to provide N-(8-((2,2-difluoroethyl)amino)-5-(5-methoxybenzo[,]oxazol-2-yl)-2,7-naphthyridin-3-yl)cyclopropanecarboxamide as a brown solid (7.2 mg, 3.2%). LCMS (ESI) m/z 440.1, [M+H]⁺. NMR (400 MHz, DMSO-d₆) δ 11.13 (s, 114), 9.68 (s, 1H), 9.55 (s, 1H), 8.89 (s, 1H), 8.91-8.86 (m, 1H), 7.64 (d, J=8.8 Hz, 1H), 7.31 (d, J=2.4 Hz, 1H), 6.98 (dd, J=8.8, 2.4 Hz, 1H), 6.49-6.11 (m, 1H), 4.12-3.97 (m, 2H), 3.86 (s, 3H), 2.19-2.04 (m, 1H), 0.98-0.79 (m, 4H).

### Example 371: Synthesis of N-(5-(benzo[d]oxazol-2-yl)-8-hydroxy-2,7-naphthyridin-3-yl)cyclopropanecarboxamide

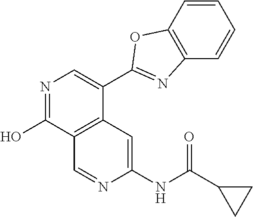 [SMILES: O=C(Nc1cc2c(-c3nc4ccccc4o3)cnc(O)c2cn1)C1CC1]

### Step 1: 6-chloro-4-iodo-2,7-naphthyridin-1-ol

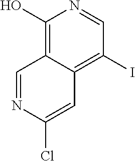 [SMILES: Oc1ncc(I)c2cc(Cl)ncc12]

To a stirred solution of 6-chloro-2,7-naphthyridin-1-ol (1.05 g; 5.540 mmol; 1.00 eq.) in DMF (20 mL) was added NIS (1.87 g; 8.312 mmol; 1.50 eq.) in portions at 0° C. under nitrogen atmosphere. The mixture was stirred at room temperature for 3 hours. The desired product was detected via LCMS. The precipitated solids were collected by filtration and washed with CH₂Cl₂ (2×10 mL). The solids were dried under vacuum to afford 6-chloro-4-iodo-2,7-naphthyridin-1-ol as an off-white solid (1.47 g, 86.6%). LCMS (ESI) ink 306.9, [M+H]⁺.

### Step 2: 4-(benzo[d]oxazol-2-yl)-6-chloro-2,7-naphthyridin-1-ol

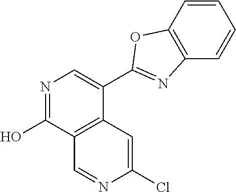 [SMILES: Oc1ncc(-c2nc3ccccc3o2)c2cc(Cl)ncc12]

To a solution of 6-chloro-4-iodo-2,7-naphthyridin-1-ol (1.12 g; 3.654 mmol, 1.00 eq.), CuI (0.10 g; 0.548 mmol; 0.15 eq.), Pd(PPh₃)²Cl₂ (0.51 g; 0.731 mmol; 0.20 eq.) in 1,4-dioxane (24 mL) was added 2-(tributylstannyl)benzo[d]oxazole (7.46 g; 18.270 mmol; 5.00 eq.). The mixture was stirred at 80° C. for 1 hour under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide 4-(benzo[d]oxazol-2-yl)-6-chloro-2,7-naphthyridin-1-ol as a brown solid (700.0 mg, 64.3%). LCMS (ESI) m/z 298.0, [M+H]⁺.

### Step 3: N-(5-(benzo[d]oxazol-2-yl)-8-hydroxy-2,7-naphthyridin-3-yl)cyclopropanecarboxamide

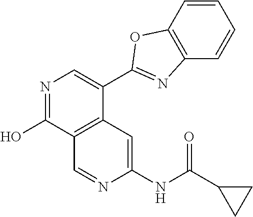 [SMILES: O=C(Nc1cc2c(-c3nc4ccccc4o3)cnc(O)c2cn1)C1CC1]

A stirred solution of 4-(benzo[d]oxazol-2-yl)-6-chloro-2,7-naphthyridin-1-ol (50.0 mg; 0.168 mmol, 1.00 eq.), Pd₂(dba)₃ (15.4 mg; 0.017 mmol; 0.10 eq.), Xantphos (9.7 mg; 0.017 mmol, 0.10 eq.), Cs₂CO₃ (164.2 mg; 0.504 mmol; 3.00 eq.) and cyclopropanecarboxamide (42.7 mg; 0.504 mmol; 3.00 eq.) in 1,4-dioxane (5 mL) was stirred at 130° C. for 14 hours under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide the crude product. The crude product was purified by flash chromatography on pre-packed C18 column using 0-40% of MeOH in water (10 mmol/L NH₄HCO₃) as eluent to provide N-(5-(benzo[d]oxazol-2-yl)-8-hydroxy-2,7-naphthyridin-3-yl)cyclopropanecarboxamide as a yellow solid (3.2 mg, 5.4%). LCMS (ESI) m/z 347.1, [M+H]⁺.

### Example 372: Synthesis of 4-(5-methoxybenzo(dioxazol-2-yl)-N¹-methyl-N⁶-(pyridin-2-yl)-2,7-naphthyridine-1,6-diamine

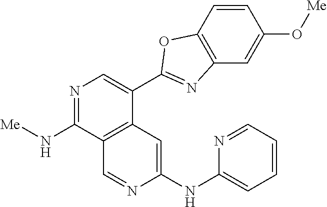 [SMILES: CNc1ncc(-c2nc3cc(OC)ccc3o2)c2cc(Nc3ccccn3)ncc12]

### Step 1: 5-methoxybenzo[d]oxazole

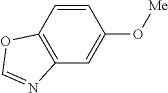 [SMILES: COc1ccc2ocnc2c1]

A solution of 2-amino-4-methoxyphenol (6.00 g; 43.118 mmol; 1.00 eq.) in trimethoxymethane (50 mL) was stirred at 100° C. for 3 hours under nitrogen atmosphere. The desired product was detected via LCMS. The mixture was concentrated under vacuum. The residue was purified by flash chromatography on silica gel column using 10-20% of EtOAc in petroleum ether to afford 5-methoxybenzo[d]oxazole as an off-white solid (6.2 g, 96.4%). LCMS (ESI) m/z 150.1, [M+H]⁺.

### Step 2: N-(5-(5-methoxybenzo[d]oxazol-2-yl)-8-(methylamino)-2,7-naphthyridin-3-yl)cyclopropanecarboxamide

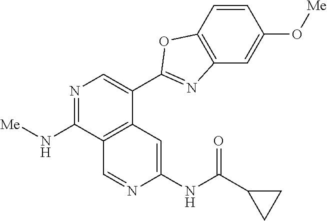 [SMILES: CNc1ncc(-c2nc3cc(OC)ccc3o2)c2cc(NC(=O)C3CC3)ncc12]

A solution of N-(5-bromo-8-(methylamino)-2,7-naphthyridin-3-yl)cyclopropanecarboxamide (500.0 mg; 1.557 mmol; 1.00 eq.), 5-methoxybenzo[d]oxazole (278.6 mg; 1.868 mmol; 1.20 eq.), Pd(PPh₃)₄ (107.9 mg; 0.093 mmol; 0.06 eq.) and Cs₂CO₃ (1521.7 mg; 4.670 mmol; 3.00 eq.) in DMF (10 mL) was stirred at 110° C. overnight under nitrogen atmosphere. The desired product was detected via LCMS. The mixture was diluted with EtOAc (3×100 mL) and washed with brine (3×10 mL), dried over anhydrous Na₂SO₄, filtered, and concentrated under reduced pressure. The residue was purified by flash chromatography on silica gel column using 2-10% of MeOH in CH₂Cl₂ to afford N-(5-(5-methoxybenzo[d]oxazol-2-yl)-8-(methylamino)-2,7-naphthyridin-3-yl)cyclopropanecarboxamide as a yellow solid (550.0 mg, 90.9%). LCMS (ESI) m/z 390.1, [M+H]⁺.

### Step 3: 4-(5-methoxybenzo[d]oxazol-2-yl)-N¹-methyl-2,7-naphthyridine-1,6-diamine

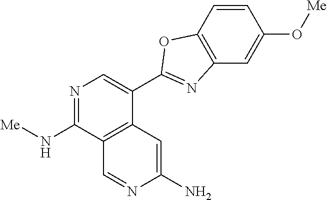 [SMILES: CNc1ncc(-c2nc3cc(OC)ccc3o2)c2cc(N)ncc12]

To a stirred solution of N-(5-(5-methoxybenzo[d]oxazol-2-yl)-8-(methylamino)-2,7-naphthyridin-3-yl)cyclopropanecarboxamide (550.0 mg; 1.412 mmol; 1.00 eq.) in a mixture solvent of DMSO/MeOH (1:5, 30 mL) was added NaOH (1.9 M in water, 7.5 mL) at room temperature. The resulting solution was stirred at 60° C. overnight. The mixture was concentrated under vacuum to remove MeOH. To this mixture was added water (100 mL), and a large amount of precipitated product was formed. The precipitated product was collected by filtration and washed with water (3×100 mL) to afford 4-(5-methoxybenzo[d]oxazol-2-yl)-N¹-methyl-2,7-naphthyridine-1,6-diamine as a yellow solid (400.0 mg, 88.1%). LCMS (ER) m/z 322.3, [M+H]⁺.

### Step 4: 4-(5-methoxybenzo[d]oxazol-2-yl)-N¹-methyl-N⁶-(pyridin-2-yl)-2,7-naphthyridine-1,6-diamine

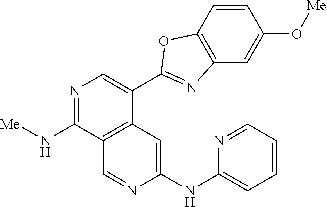 [SMILES: CNc1ncc(-c2nc3cc(OC)ccc3o2)c2cc(Nc3ccccn3)ncc12]

A solution of 4-(5-methoxybenzo[d]oxazol-2-yl)-N¹-methyl-2,7-naphthyridine-1,6-diamine (80.0 mg; 0.249 mmol; 1.00 eq.), 2-chloropyridine (28.3 mg; 0.249 mmol; 1.00 eq.), Pd₂(dba)₃ (27.4 mg; 0.030 mmol; 0.12 eq.), XantPhos (34.6 mg; 0.060 mmol; 0.24 eq.), Cs₂CO₃ (162.2 mg; 0.498 mmol; 2.00 eq.) in 1,4-dioxane (5 mL) was stirred at 130° C. for 5 hours under nitrogen atmosphere. The desired product was detected via LCMS. The mixture was concentrated under reduced pressure. The residue was purified by flash chromatography on silica gel column using 10-20% of MeOH in CH₂Cl₂ as eluent to provide a crude product. The crude product was purified by flash chromatography on pre-packed C18 column using 20-70% of MeCN in water (10 mmol/L NH₄HCO₃) to provide 4-(5-methoxybenzo[d]oxazol-2-yl)-N¹-methyl-N⁶-(pyridin-2-yl)-2,7-naphthyridine-1,6-diamine as a yellow solid (41.6 mg, 41.1%). LCMS (ESI) m/z 399.1, [M+H]⁺. ¹H NMR (400 MHz, DMSO-d₆) δ 10.09 (s, 1H), 9.71 (s, 1H), 9.37 (s, 1H), 8.87 (s, 1H), 8.60-8.49 (m, 1H), 8.48-8.31 (m, 1H), 7.76-7.69 (m, 1H), 7.65 (d, J=8.4 Hz, 1H), 7.45 (d, J=8.4 1H), 7.32 (d, J=2.4 Hz, 1H), 7.03-6.88 (m, 2H), 3.87 (s, 3H), 3.09 (d, J=4.4 Hz, 3H).

### Example 373: Synthesis of N-(5-(5-(2-hydroxy-2-methylpropoxy)benzo[d]oxazol-2-yl)-8-(methylamino)-2,7-naphthyridin-3-yl)cyclopropanecarboxamide

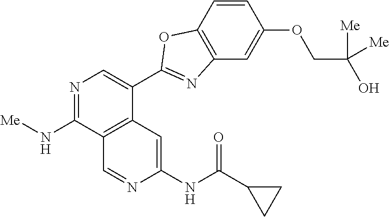 [SMILES: CNc1ncc(-c2nc3cc(OCC(C)(C)O)ccc3o2)c2cc(NC(=O)C3CC3)ncc12]

### Step 1: 1-(4-(benzyloxy)phenoxy)-2-methylpropan-2-ol

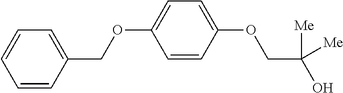 [SMILES: CC(C)(O)COc1ccc(OCc2ccccc2)cc1]

A mixture of 1-bromo-2-methylpropan-2-ol (764.1 mg; 4.994 mmol; 2.00 eq.), 4-(benzyloxy)phenol (500.0 mg; 2,497 mmol; 1.00 eq.) and Cs₂CO₃ (1.62 g; 4.994 mmol; 2.00 eq.) in DMF (5 mL) was stirred at 120° C. for 12 hours. The desired product was detected via LCMS. The solids were filtered out and the filtrate was concentrated under reduced pressure. The residue was purified by flash chromatography on pre-packed. C18 column using 50-70% of MeCN in water (10 mmol/L, NH₄HCO₃) as eluent to provide 1-(4-(benzyloxy)phenoxy)-2-methylpropan-2-ol as a white solid (340.0 mg, 50.0%). LCMS (ESI) m/z 271.1, [M+H]⁺.

### Step 2: 4-(2-hydroxy-2-methylpropoxy)phenol

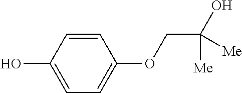 [SMILES: CC(C)(O)COc1ccc(O)cc1]

To a stirring mixture of 1-(4-(benzyloxy)phenoxy)-2-methylpropan-2-ol (340.0 mg; 1.248 mmol; 1.00 eq.) in MeOH (30 mL) was added 10% Pd/C (68.0 mg; 20% w/w) under nitrogen atmosphere. The mixture was hydrogenated at room temperature overnight under hydrogen atmosphere (2 atm). The resulting mixture was filtered, the filter cake was washed with MeOH (3×10 mL). The filtrate was concentrated under reduced pressure. The residue was purified by flash chromatography on pre-packed C18 column using 30-60% of MeCN in water (10 mmol/L NH₄HCO₃) as eluent to provide 4-(2-hydroxy-2-methylpropoxy)phenol as a white solid (200.0 mg, 87.9%). LCMS (ESI) m/z 181.1, [M−H]⁻.

### Step 3: 4-(2-hydroxy-2-methylpropoxy)-2-nitrophenol

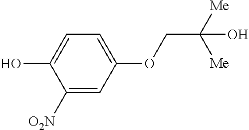 [SMILES: CC(C)(O)COc1ccc(O)c([N+](=O)[O-])c1]

A mixture of 4-(2-hydroxy-2-methylpropoxy)phenol (200.0 mg; 1.098 mmol, 1.00 eq.) and urea nitrate (135.0 mg; 1.098 mmol; 1.00 eq.) in MeCN/water (19:1, 5 mL) was stirred at 80° C. overnight. The desired product was detected via LCMS. The resulting mixture was concentrated under reduced pressure. The residue was purified by flash chromatography on pre-packed C18 column using 30-60% of MeCN in water (10 mmol/L NH₄HCO₃) as eluent to provide 4-(2-hydroxy-2-methylpropoxy)-2-nitrophenol as a black solid (140.0 mg, 56.1%). LCMS (ESI) m/z 226.1, [M−H]⁻.

### Step 4: 2-amino-4-(2-hydroxy-2-methylpropoxy)phenol

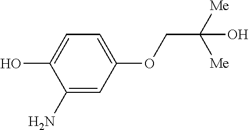 [SMILES: CC(C)(O)COc1ccc(O)c(N)c1]

To a stirring mixture of 4-(2-hydroxy-2-methylpropoxy)-2-nitrophenol (120.0 mg; 0.528 mmol; 1.00 eq.) in MeOH (10 mL) was added 10% Pd/C (24.0 Trig; 20% w/w) under nitrogen atmosphere. The mixture was hydrogenated at room temperature overnight under hydrogen atmosphere (2 atm). The desired product was detected via LCMS. The resulting mixture was filtered, the filter cake was washed with MeOH (3×5 mL). The filtrate was concentrated under reduced pressure. The residue was purified by flash chromatography on pre-packed C18 column using 35-55% of MeCN in water (10 mmol/L NH₄HCO₃) as eluent to provide 2-amino-4-(2-hydroxy-2-methylpropoxy)phenol as a black solid (80.0 mg, 76.8%). LCMS (ER) m/z 198.1, [M+H]⁺.

### Step 5: N-(5-(5-(2-hydroxy-2-methylpropoxy)benzo[d]oxazol-2-yl)-8-(methylamino)-2,7-naphthyridin-3-yl)cyclopropanecarboxamide

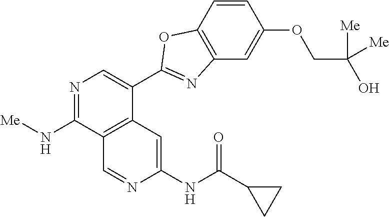 [SMILES: CNc1ncc(-c2nc3cc(OCC(C)(C)O)ccc3o2)c2cc(NC(=O)C3CC3)ncc12]

N-(5-(5-(2-hydroxy-2-methylpropoxy)benzo[d]oxazol-2-yl)-8-(methylamino)-2,7-naphthyridin-3-yl)cyclopropanecarboxamide was synthesized using a similar procedure that was previously described in Example 210, by using N-(5-formyl-8-(methylamino)-2,7-naphthyridin-3-yl)cyclopropanecarboxamide and 2-amino-4-(2-hydroxy-2-methylpropoxy)phenol as the starting material. LCMS (ESI) m/z 448.2, [M+H]⁺. ¹H NMR (300 MHz, DMSO-d₆) δ 11.05 (s, 1H), 9.67 (s, 1H), 9.43 (s, 1H), 8.88 (s, 1H), 8.70-8.61 (m, 1H), 7.60 (d, J=8.8 Hz, 1H), 7.26 (d, J=2.4 Hz, 1H), 6.97 (dd, J=8.8, 2.4 Hz, 1H), 4.66 (s, 1H), 3.81 (s, 2H), 3.08 (d, J=4.4 Hz, 3H), 2.14-2.06 (m, 1H), 1.24 (s, 6H), 0.95-0.81 (m, 4H).

### Examples 374-379

Each compound in Table 15 below was prepared using a similar experimental procedure to prepare Example 372, using 4-(5-methoxybenzo[d]oxazol-2-yl)-N¹-methyl-2,7-naphthyridine-1,6-diamine as the common intermediate and appropriate aryl halide.

TABLE 15

| Example | Structure and Name | MS and ¹H NMR |
|---|---|---|
| Example 374 | 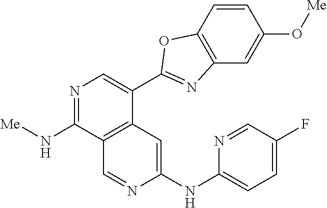 [SMILES: CNc1ncc(-c2nc3cc(OC)ccc3o2)c2cc(Nc3ccc(F)cn3)ncc12] | LCMS (ESI) m/z 417.1, [M + H]⁺. |
| | N⁶-(5-fluoropyridin-2-y1)-4-(5- | |
| | methoxybenzo[d]oxazol-2-yl)-N¹- | |
| | methyl-2,7-naphthyridine-1,6- | |
| | diamine | |
| Example 375 | 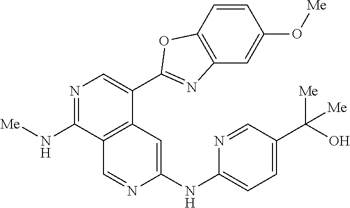 [SMILES: CNc1ncc(-c2nc3cc(OC)ccc3o2)c2cc(Nc3ccc(C(C)(C)O)cn3)ncc12] | LCMS (ESI) m/z 457.2, [M + H]⁺. ¹H NMR (400 MHz, DMSO-d₆) δ 9.99 (s, 1H), 9.67 (s, 1H), 9.35 (s, 1H), 8.84 (s, 1H), 8.52-8.47 (m, 2H), 7.79 (dd, J = 8.8, 2.4 Hz, 1H), 7.64 (d, J = 8.8 Hz, 1H), 7.41 (d, J = 8.8 Hz, 1H), 7.29 (d, J = 2.4 Hz, 1H), 6.98 (dd, J = 8.8, 2.4 Hz, 1H), 5.10 (s, 1H), 3.86 (s, 3H), 3.08 (d, J = 4.4 Hz, 3H), 1.49 (s, 6H). |
| | 2-(6-((5-(5-methoxybenzo[d]oxazol- | |
| | 2-y1)-8-(methylamino)-2,7- | |
| | naphthyridin-3-yl)amino)pyridin- | |
| | 3-yl)propan-2-ol | |
| Example 376 | 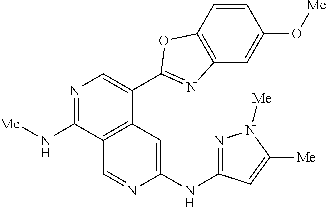 [SMILES: CNc1ncc(-c2nc3cc(OC)ccc3o2)c2cc(Nc3cc(C)n(C)n3)ncc12] | LCMS (ESI) m/z 416.2, [M + H]⁺. ¹H NMR (400 MHz, DMSO-d₆) δ 9.48 (s, 1H), 9.25 (s, 1H), 9.05 (s, 1H), 8.79 (s, 1H), 8.43-8.36 (m, 1H), 7.63 (d, J = 8.8 Hz, 1H), 7.26 (d, J = 2.4 Hz, 1H), 6.96 (dd, J = 8.8, 2.4 Hz, 1H), 6.02 (s, 1H), 3.85 (s, 3H), 3.74 (s, 3H), 3.06 (d, J = 4.4 Hz, 3H), 2.28 (s, 3H). |
| | N⁶-(1,5-dimethyl-1H-pyrazol-3-yl)- | |
| | 4-(5-methoxybenzo[d]oxazol-2-yl)- | |
| | N¹-methyl-2,7-naphthyridine-1,6- | |
| | diamine | |
| Example 377 | 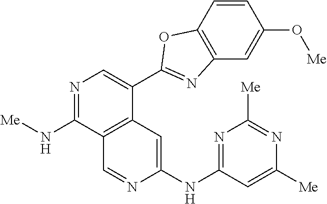 [SMILES: CNc1ncc(-c2nc3cc(OC)ccc3o2)c2cc(Nc3cc(C)nc(C)n3)ncc12] | LCMS (ESI) m/z 428.2, [M + H]⁺. ¹H NMR (400 MHz, DMSO-d₆) δ 10.37 (s, 1H), 9.92 (s, 1H), 9.41 (s, 1H), 8.89 (s, 1H), 8.69 -8.32 (m, 1H), 7.66 (d, J = 8.8 Hz, 1H), 7.28 (d, J = 2.4 Hz, 1H), 7.13-6.80 (m, 2H), 3.85 (s, 3H), 3.09 (d, J = 4.4 Hz, 3H), 2.64 (s, 3H), 2.34 (s, 3H). |
| | N⁶-(2,6-dimethylpyrimidin-4-yl)-4- | |
| | (5-methoxybenzo[d]oxazol-2-yl)- | |
| | N¹-methyl-2,7-naphthyridine-1,6- | |
| | diamine | |
| Example 378 | 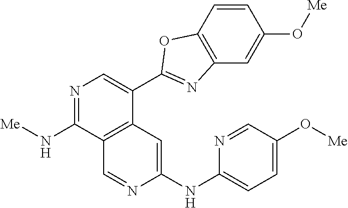 [SMILES: CNc1ncc(-c2nc3cc(OC)ccc3o2)c2cc(Nc3ccc(OC)cn3)ncc12] | LCMS (ESI) m/z 429.2, [M + H]⁺. ¹H NMR (400 MHz, DMSO-d₆) δ 9.90 (s, 1H), 9.48 (s, 1H), 9.33 (s, 1H), 8.83 (s, 1H), 8.50-8.44 (m, 1H), 8.11 (d, J = 2.8 Hz, 1H), 7.69-7.61 (m, 1H), 7.50-7.41 (m, 2H), 7.33 (d, J = 2.4 Hz, 1H), 7.01-6.93 (m, 1H), 3.87 (s, 3H), 3.85 (s, 3H), 3.08 (d, J = 4.4 Hz, 3H). |
| | 4-(5-methoxybenzo[d]oxazol-2-yl)- | |
| | N⁶-(5-methoxypyridin-2-yl)-N¹- | |
| | methyl-2,7-naphthyridine-1,6- | |
| | diamine | |
| Example 379 | 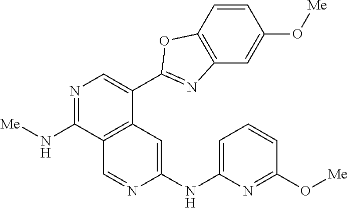 [SMILES: CNc1ncc(-c2nc3cc(OC)ccc3o2)c2cc(Nc3cccc(OC)n3)ncc12] | LCMS (ESI) m/z 429.2, [M + H]⁺. ¹H NMR (400 MHz, DMSO-d₆) δ 10.01 (s, 1H), 9.43-9.33 (m, 2H), 8.84 (s, 1H), 8.59-8.49 (m, 1H), 7.68-7.59 (m, 2H), 7.25 (d, J = 2.4 Hz, 1H), 7.01- 6.91 (m, 2H), 6.36 (d, J = 8.0 Hz, 1H), 3.85 (s, 6H), 3.09 (d, J = 4.4 Hz, 3H). |
| | 4-(5-methoxybenzo[d]oxazol-2-yl)- | |
| | N⁶-(6-methoxypyridin-2-yl)-N¹. | |
| | methyl-2,7-naphthyridine-1,6- | |
| | diamine | |

### Example 380: Synthesis of 4-(5-methoxybenzo[d]oxazol-2-yl)-N¹-methyl-N⁶-(5-morpholinopyridin-2-yl)-2,7-naphthyridine-1,6-diamine

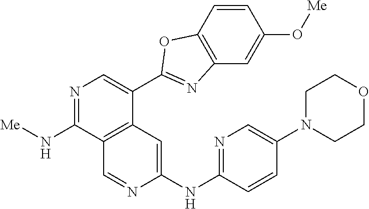 [SMILES: CNc1ncc(-c2nc3cc(OC)ccc3o2)c2cc(Nc3ccc(N4CCOCC4)cn3)ncc12]

### Step 1: 4-(6-chloropyridin-3-yl)morpholine

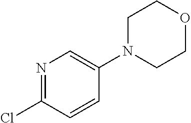 [SMILES: Clc1ccc(N2CCOCC2)cn1]

To a stirred solution of 2-chloro-5-iodopyridine (500.0 mg; 2.598 mmol; 1.00 eq.), Pd₂(dba)₃ (95.6 mg; 0.104 mmol; 0.05 eq.), XantPhos (120.8 mg; 0.209 mmol; 0.10 eq.) and i-BuOK (351.5 mg; 3.132 mmol; 1.50 eq.) in toluene (10 mL) was added morpholine (236.5 mg; 2,715 mmol; 1.30 eq.) at room temperature under nitrogen atmosphere. The resulting mixture was stirred at 100° C. for 4 hours. The resulting mixture was concentrated under reduced pressure. The residue was purified by flash chromatography on silica gel column using 0-25% of EtOAc in petroleum ether as eluent to provide 4-(6-chloropyridin-3-yl)morpholine as a yellow solid (400.0 mg, 92.6%). LCMS (ESI) m/z 199.1, [M+H]⁺.

### Step 2: 4-(5-methoxybenzo[d]oxazol-2-yl)-N¹-methyl-N⁶-(5-morpholinopyridin-2-yl)-2,7-naphthyridine-1,6-diamine

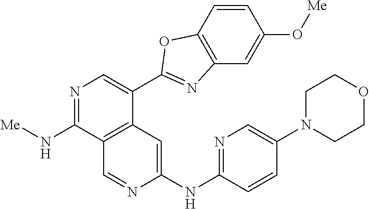 [SMILES: CNc1ncc(-c2nc3cc(OC)ccc3o2)c2cc(Nc3ccc(N4CCOCC4)cn3)ncc12]

4-(5-methoxybenzo[d]oxazol-2-yl)-N¹-methyl-N⁶-(5-morpholinopyridin-2-yl)-2,7-naphthyridine-1,6-diamine was synthesized using a similar procedure that was previously described in Example 372, by using 4-(5-methoxybenzo[d]oxazol-2-yl)-N¹-methyl-2,7-naphthyridine-1,6-diamine and 4-(6-chloropyridin-3-yl)morpholine as the starting material. LCMS (ESI) m/z 484.2, [M+H]⁺. ¹H NMR (400 MHz, DMSO-d₆) δ 9.82 (s, 1H), 9.45 (s, 1H), 9.32 (s, 1H), 8.82 (s, 1H), 8.49-8.41 (m, 1H), 8.06 (d, J=2.8 Hz, 1H), 7.64 (d, J=8.8 Hz, 1H), 7.51-7.39 (m, 2H), 7.31 (d, J=2.4 Hz, 1H), 7.00-6.96 (m, 1H), 3.86 (s, 3H), 3.81-3.76 (m, 4H), 3.19-3.11 (m, 4H), 3.08 (d, J=4.4 Hz, 3H).

### Example 381: Synthesis of 1-fluoro-N-(5-(5-methoxybenzo[d]oxazol-2-yl)-8-(methylamino)-2,7-naphthyridin-3-yl)cyclopropane-1-carboxamide

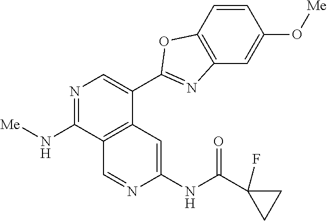 [SMILES: CNc1ncc(-c2nc3cc(OC)ccc3o2)c2cc(NC(=O)C3(F)CC3)ncc12]

To a solution of 4-(5-methoxybenzo[d]oxazol-2-yl)-N¹-methyl-2,7-naphthyridine-1,6-diamine (80.0 mg; 0.249 mmol; 1.00 eq.) and 1-fluorocyclopropane-1-carboxylic acid (23.3 mg; 0.224 mmol; 0.90 eq.) in pyridine (5 mL) was added POCl₃ (114.5 mg; 0.747 mmol; 3.00 eq.) dropwise at 0° C. The resulting mixture was stirred at room temperature for 1 hour. The desired product was detected via LCMS. The solution was quenched by the addition of water (10 mL), extracted with EtOAc (3×50 mL). The combined organic layers were washed with brine (3×10 mL), dried over anhydrous Na₂SO₄, filtered, and concentrated under reduced pressure. The residue was purified by flash chromatography on silica gel column using 5-10% of MeOH in CH₂Cl₂ as eluent to afford a crude product. The crude product was purified by flash chromatography on pre-packed C18 column using 30-60% of MeOH in water (10 mmol/L NH₄HCO₃) as eluent to provide 1-fluoro-N-(5-(5-methoxybenzo[d]oxazol-2-yl)-8-(methylamino)-2,7-naphthyridin-3-yl)cyclopropane-1-carboxamide as a yellow solid (45.4 mg, 43.4%). LCMS (ESI) m/z 408.1 [M+H]⁺. ¹H NMR (400 MHz, DMSO-d₆) δ 10.44 (s, 1H), 9.68 (s, 1H), 9.50 (s, 1H), 8.94 (s, 1H), 8.80-8.70 (m, 1H), 7.64 (d, J=10.0 Hz, 1H), 7.29 (d, J=2.4 Hz, 1H), 7.00-6.94 (m, 1H), 3.85 (s, 3H), 3.11 (d, J=4.4 Hz, 3H), 1.52-1.42 (m, 4H).

### Examples 382-392

Each compound in Table 15 below was prepared using a similar experimental procedure to prepare Example 381, using 4-(5-methoxybenzo[d]oxazol-2-yl)-N¹-methyl-2,7-naphthyridine-1,6-diamine as the common intermediate and appropriate acid.

TABLE 15

| Example | Structure and Name | MS and ¹H NMR |
|---|---|---|
| Example 382 | 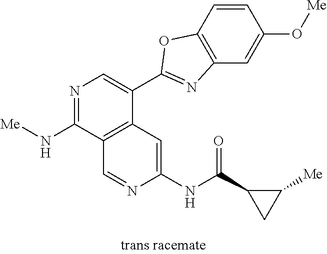 [SMILES: CNc1ncc(-c2nc3cc(OC)ccc3o2)c2cc(NC(=O)[C@@H]3C[C@H]3C)ncc12] | LCMS (ESI) m/z 404.2, [M + H]⁺. ¹H NMR (400 MHz, DMSO-d₆) δ 10.93 (s, 1H), 9.62 (s, 1H), 9.43 (s, 1H), 8.89 (s, 1H), 8.65-8.60 (m, 1H), 7.62 (d, J = 8.8 Hz, 1H), 7.28 (s, 1H), 6.95 (d, J = 8.8 Hz, 1H), 3.86 (s, 3H), 3.09 (d, J = 4.4 Hz, 3H), 1.90 - 1.81 (m, 1H), 1.37-1.30 (m, 1H), 1.19-1.06 (m, 4H), 0.76-0.67 (m, 1H). |
| | N-(5-(5-methoxybenzo[d]oxazol-2- | |
| | yl)-8-(methylamino)-2,7- | |
| | naphthyridin-3-y1)-2- | |
| | methylcyclopropane-1~ | |
| | carboxamide (trans racemate) | |
| Example 383 | 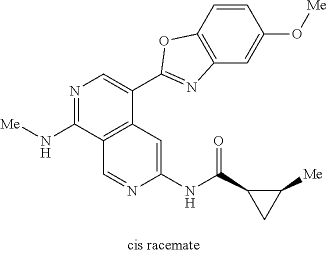 [SMILES: CNc1ncc(-c2nc3cc(OC)ccc3o2)c2cc(NC(=O)[C@@H]3C[C@@H]3C)ncc12] | LCMS (ESI) m/z 404.2, [M + H]⁺. ¹H NMR (400 MHz, DMSO-d₆) δ 10.91 (s, 1H), 9.62 (s, 1H), 9.43 (s, 1H), 8.89 (s, 1H), 8.66-8.60 (m, 1H), 7.62 (d, J = 8.8 Hz, 1H), 7.29 (s, 1H), 6.97 (d, J = 8.8 Hz, 1H), 3.86 (s, 3H), 3.09 (d, J = 4.4 Hz, 3H), 2.16-2.06 (m, 1H), 1.36-1.26 (m, 1H), 1.17 (d, J = 6.2 Hz, 3H), 1.05-0.97 (m, 1H), 0.92- 0.85 (m, 1H). |
| | N-(5-(5-methoxybenzo[d]oxazol-2- | |
| | yl)-8-(methylamino)-2,7- | |
| | naphthyridin-3-y1)-2- | |
| | methylcyclopropane-1- | |
| | carboxamide (cis racemate) | |
| Example 384 | 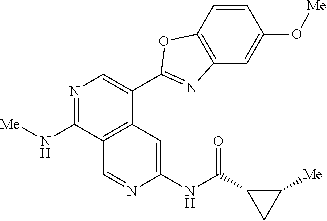 [SMILES: CNc1ncc(-c2nc3cc(OC)ccc3o2)c2cc(NC(=O)[C@H]3C[C@H]3C)ncc12] | LCMS (ESI) m/z 404.2. ¹H NMR (400 MHz, DMSO-d₆) δ 10.92 (s, 1H), 9.62 (s, 1H), 9.43 (s, 1H), 8.89 (s, 1H), 8.67-8.59 (m, 1H), 7.62 (d, J = 8.8 Hz, 1H), 7.29 (d, J = 2.4 Hz, 1H), 7.00-6.93 (m, 1H), 3.86 (s, 3H), 3.09 (d, J = 4.4 Hz, 3H), 2.16-2.06 (m, 1H), 1.38-1.26 (m, 1H), 1.17 (d, J = 6.1 Hz, 3H), 1.07-0.97 (m, 1H), 0.93-0.85 (m, 1H). |
| | (1S,2R)-N-(5-(5- | |
| | methoxybenzo[d]oxazol-2-yl)-8- | |
| | (methylamino)-2,7-naphthyridin-3- | |
| | y1)-2-methylcyclopropane-1- | |
| | carboxamide | |
| Example 385 | 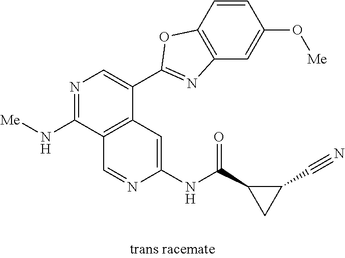 [SMILES: CNc1ncc(-c2nc3cc(OC)ccc3o2)c2cc(NC(=O)[C@@H]3C[C@H]3C#N)ncc12] | LCMS (ESI) m/z 415.1, [M + H]⁺. ¹H NMR (400 MHz, DMSO-d₆) δ 11.34 (s, 1H), 9.66 (s, 1H), 9.47 (s, 1H), 8.93 (s, 1H), 8.75-8.65 (m, 1H), 7.62 (d, J = 8.8 Hz, 1H), 7.27 (d, J = 2.0 Hz, 1H), 7.01-6.89 (m, 1H), 3.85 (s, 3H), 3.10 (d, J = 4.4 Hz, 3H), 2.64- 2.59 (m, 1H), 2.36-2.30 (m, 1H), 1.63-1.54 (m, 1H), 1.54-1.42 (m, 1H). |
| | 2-cyano-N-(5-(5- | |
| | methoxybenzo[d]oxazol-2-y1)-8- | |
| | (methylamino)-2,7-naphthyridin-3- | |
| | yl)cyclopropane-1-carboxamide | |
| | (trans racemate) | |
| Example 386 | 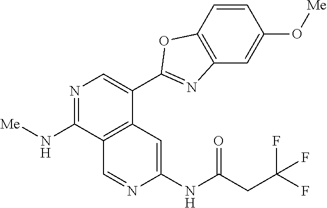 [SMILES: CNc1ncc(-c2nc3cc(OC)ccc3o2)c2cc(NC(=O)CC(F)(F)F)ncc12] | LCMS (ESI) m/z 432.1, [M + H]⁺. ¹H NMR (400 MHz, DMSO-d₆) δ 11.10 (s, 1H), 9.69 (s, 1H), 9.46 (s, 1H), 8.93 (s, 1H), 8.71-8.67 (m, 1H), 7.63 (d, J = 8.8 Hz, 1H), 7.29 (d, J = 2.4 Hz, 1H), 6.97 (d, J = 8.8, 2.4 Hz, 1H), 3.86 (s, 3H), 3.75-3.67 (m, 2H), 3.10 (d, J = 4.4 Hz, 3H). |
| | 3,3,3-trifluoro-N-(5-(5- | |
| | methoxybenzo[d]oxazol-2-yl)-8- | |
| | (methylamino)-2,7-naphthyridin-3- | |
| | yl)propanamide | |
| Example 387 | 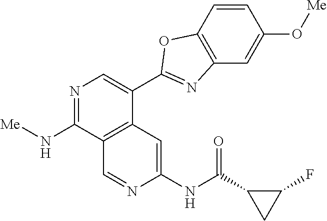 [SMILES: CNc1ncc(-c2nc3cc(OC)ccc3o2)c2cc(NC(=O)[C@H]3C[C@H]3F)ncc12] | LCMS (ESI) m/z 408.1 [M + H]⁺. ¹H NMR (400 MHz, DMSO-d₆) δ 11.09 (s, 1H), 9.66 (s, 1H), 9.45 (s, 1H), 8.91 (s, 1H), 8.70-8.59 (m, 1H), 7.63 (d, J = 8.8 Hz, 1H), 7.30 (d, J = 2.4 Hz, 1H), 7.00-6.90 (m, 1H), 5.10- 4.86 (m, 1H), 3.86 (s, 3H), 3.09 (d, J = 4.4 Hz, 3H), 2.37-2.25 (m, 1H), 1.81-1.67 (m, 1H), 1.29-1.16 (m, 1H). |
| | (1R,2R)-2-fluoro-N-(5-(5- | |
| | methoxybenzo[d]oxazol-2-y1)-8- | |
| | (methylamino)-2,7-naphthyridin-3- | |
| | yl)cyclopropane-1-carboxamide | |
| Example 388 | 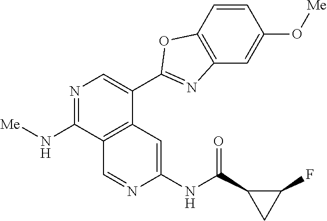 [SMILES: CNc1ncc(-c2nc3cc(OC)ccc3o2)c2cc(NC(=O)[C@@H]3C[C@@H]3F)ncc12] | LCMS (ESI) m/z 408.1. ¹H NMR (400 MHz, DMSO-d₆) δ 11.08 (s, 1H), 9.66 (s, 1H), 9.45 (s, 1H), 8.91 (s, 1H), 8.70-8.62 (m, 1H), 7.63 (d, J = 8.9 Hz, 1H), 7.30 (d, J = 2.6 Hz, 1H), 7.02-6.93 (m, 1H), 5.18-4.79 (m, 1H), 3.86 (s, 3H), 3.10 (d, J = 4.4 Hz, 3H), 2.35-2.25 (m, 1H), 1.82- 1.67 (m, 1H), 1.29-1.16 (m, 1H). |
| | (1S,2S)-2-fluoro-N-(5-(5- | |
| | methoxybenzo[d]oxazol-2-y1)-8- | |
| | (methylamino)-2,7-naphthyridin-3- | |
| | yl)cyclopropane-1-carboxamide | |
| Example 389 | 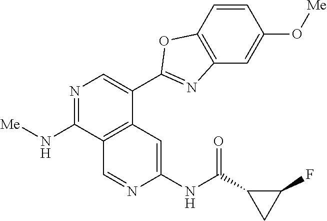 [SMILES: CNc1ncc(-c2nc3cc(OC)ccc3o2)c2cc(NC(=O)[C@H]3C[C@@H]3F)ncc12] | LCMS (ESI) m/z 408.1 [M + H]⁺. ¹H NMR (400 MHz, DMSO-d₆) δ 11.22 (s, 1H), 9.65 (s, 1H), 9.46 (s, 1H), 8.88 (s, 1H), 8.82-8.71 (m, 1H), 7.62 (d, J = 8.8 Hz, 1H), 7.27 (d, J= 2.4 Hz, 1H), 7.00-6.95 (m, 1H), 5.10- 4.86 (m, 1H), 3.86 (s, 3H), 3.10 (d, J = 4.4 Hz, 3H), 2.71-2.59 (m, 1H), 1.65-1.52 (m, 1H), 1.40-1.30 (m, 1H). |
| | (1R,2S)-2-fluoro-N-(5-(5- | |
| | methoxybenzo[d]oxazol-2-yl)-8- | |
| | (methylamino)-2,7-naphthyridin-3- | |
| | yl)cyclopropane-1-carboxamide | |
| Example 390 | 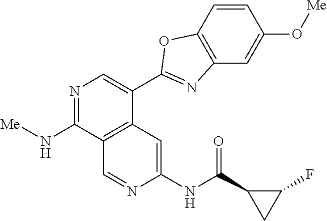 [SMILES: CNc1ncc(-c2nc3cc(OC)ccc3o2)c2cc(NC(=O)[C@@H]3C[C@H]3F)ncc12] | LCMS (ESI) m/z 408.1. ¹H NMR (400 MHz, DMSO-d₆) δ 11.21 (s, 1H), 9.64 (s, 1H), 9.45 (s, 1H), 8.91 (s, 1H), 8.70-8.62 (m, 1H), 7.62 (d, J = 8.8 Hz, 1H), 7.26 (d, J = 2.5 Hz, 1H), 7.00-6.92 (m, 1H), 5.17-4.77 (m, 1H), 3.85 (s, 3H), 3.09 (d, J = 4.5 Hz, 3H), 2.71-2.58 (m, 1H), 1.67- 1.48 (m, 1H), 1.41-1.28 (m, 1H). |
| | (1S,2R)-2-fluoro-N-(5-(5- | |
| | methoxybenzo[d]oxazol-2-yl)-8- | |
| | (methylamino)-2,7-naphthyridin-3- | |
| | yl)cyclopropane-1-carboxamide | |
| Example 391 | 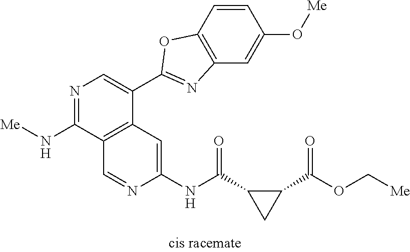 [SMILES: CCOC(=O)[C@@H]1C[C@@H]1C(=O)Nc1cc2c(-c3nc4cc(OC)ccc4o3)cnc(NC)c2cn1] | LCMS (ESI) m/z 462.2, [M + H]⁺. ¹H NMR (400 MHz, DMSO-d₆) δ 11.03 (s, 1H), 9.60 (s, 1H), 9.44 (s, 1H), 8.90 (s, 1H), 8.74-8.51 (m, 1H), 7.61 (d, J = 9.2 Hz, 1H), 7.22 (d, J = 2.4 Hz, 1H), 7.08-6.81 (m, 1H), 4.10- 4.01 (m, 2H), 3.85 (s, 3H), 3.09 (d, J = 4.4 Hz, 3H), 2.48-2.43 (m, 1H), 2.24-2.18 (m, 1H), 1.54-1.49 (m, 1H), 1.29-1.22 (m, 1H), 1.17-1.11 (m, 3H). |
| | ethyl 2-((5-(5- | |
| | methoxybenzo[d]oxazol-2-yl)-8- | |
| | (methylamino)-2,7-naphthyridin-3- | |
| | yl)carbamoyl)cyclopropane-1- | |
| | carboxylate (cis racemate) | |
| Example 392 | 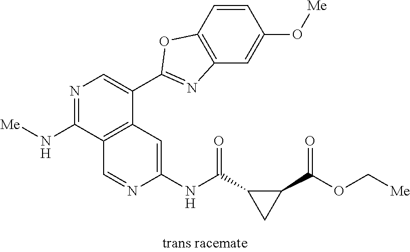 [SMILES: CCOC(=O)[C@H]1C[C@@H]1C(=O)Nc1cc2c(-c3nc4cc(OC)ccc4o3)cnc(NC)c2cn1] | LCMS (ESI) m/z 462.2, [M + H]⁺. |
| | ethyl 2-((5-(5- | |
| | methoxybenzo[d]oxazol-2-y1)-8- | |
| | (methylamino)-2,7-naphthyridin-3- | |
| | yl)carbamoyl)cyclopropane-1- | |
| | carboxylate (trans racemate) | |

Example 393, Example 394, and Example 395: Synthesis of 2-(difluoromethyl)-N-(5-(5-methoxybenzo[d]oxazol-2-yl)-8-(methylamino)-17-naphthyridin-3-yl)cyclopropane-1-carboxamide (cis racemate) (Example 393). (1R,2S)-2-(difluoromethyl)-N-(5-(5-methoxybenzo[d]oxazol-2-yl)-8-(methylamino)-17-naphthyridin-3-yl)cyclopropane-1-carboxamide (Example 394) and (1S,2R)-2-(difluoromethyl)-N-(5-(5-methoxybenzo[d]oxazol-2-yl)-8-(methylamino)-2,7-naphthyridin-3-yl)cyclopropane-1-carboxamide (Example 395)

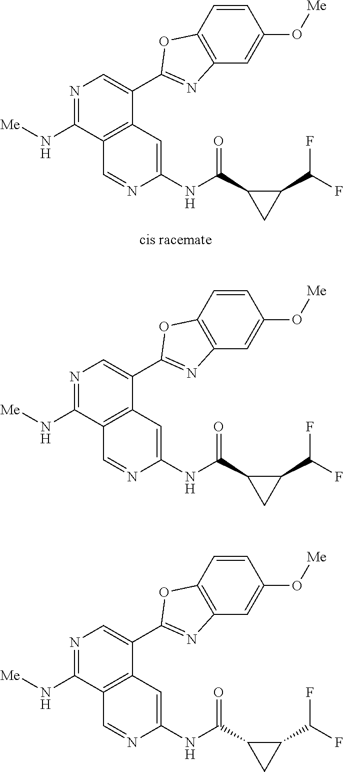 [SMILES: CNc1ncc(-c2nc3cc(OC)ccc3o2)c2cc(NC(=O)[C@@H]3C[C@@H]3C(F)F)ncc12 ; CNc1ncc(-c2nc3cc(OC)ccc3o2)c2cc(NC(=O)[C@@H]3C[C@@H]3C(F)F)ncc12 ; CNc1ncc(-c2nc3cc(OC)ccc3o2)c2cc(NC(=O)[C@H]3C[C@H]3C(F)F)ncc12]

### Step 1: 2-(difluoromethyl)-N-(5-(5-methoxybenzo[d]oxazol-2-yl)-8-(methylamino)-2,7-naphthyridin-3-yl)cyclopropane-1-carboxamide

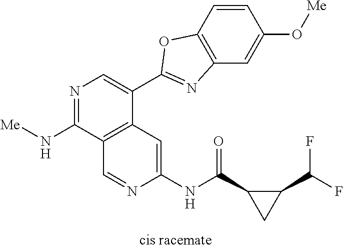 [SMILES: CNc1ncc(-c2nc3cc(OC)ccc3o2)c2cc(NC(=O)[C@@H]3C[C@@H]3C(F)F)ncc12]

2-(difluoromethyl)-N-(5-(5-methoxybenzo[d]oxazol-2-yl)-8-(methylamino)-2,7-naphthyridin-3-yl)cyclopropane-1-carboxamide (cis racemate) was synthesized using a similar procedure that was previously described in Example 381, by using 4-(5-methoxybenzo[d]oxazol-2-yl)-N¹-methyl-2,7-naphthyridine-1,6-diamine and 2-(difluoromethyl)cyclopropane-1-carboxylic acid (cis racemate) as the starting material. LCMS (ESI) m/z 440.1, [M+H]⁺. ¹H NMR (400 MHz, DMSO-d₆) δ 11.27 (s, 1H), 9.64 (s, 1H), 9.46 (s, 1H), 8.92 (s, 1H), 8.72-8.64 (m, 1H), 7.63 (d, J=8.8 Hz, 1H), 7.30 (d, J=2.5 Hz, 1H), 7.01-6.93 (m, 1H), 6.25-5.95 (m, 1H), 3.86 (s, 3H), 3.10 (d, J=4.4 Hz, 3H), 2.47-2.38 (m, 1H), 1.99-1.87 (m, 1H), 1.43-1.29 (m, 2H).

### Step 2: (1R,2S)-2-(difluoromethyl)-N-(5-(5-methoxybenzo[d]oxazol-2-yl)-8-(methylamino)-2,7-naphthyridin-3-yl) cyclopropane-1-carboxamide and (1S,2R)-2-(difluoromethyl)-N-(5-(5-methoxybenzo[d]oxazol-2-yl)-8-(methylamino)-2,7-naphthyridin-3-yl)cyclopropane-1-carboxamide

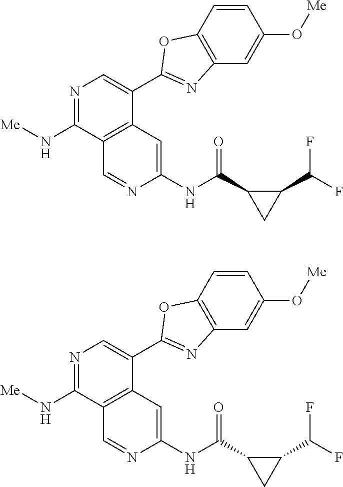 [SMILES: CNc1ncc(-c2nc3cc(OC)ccc3o2)c2cc(NC(=O)[C@@H]3C[C@@H]3C(F)F)ncc12 ; CNc1ncc(-c2nc3cc(OC)ccc3o2)c2cc(NC(=O)[C@H]3C[C@H]3C(F)F)ncc12]

2-(difluoromethyl)-N-(5-(5-methoxybenzo[d]oxazol-2-yl)-8-(methylamino)-2,7-naphthyridin-3-yl)cyclopropane-1-carboxamide (cis racemate) was separated by Prep-Chiral-HPLC (Column: CHIRAL ART Cellulose-SA, 2×25 cm, 5 um; Mobile Phase A: Hex (0.5% 2 M NH₃·MeOH), Mobile Phase B: IPA:DCM=1:1; Flow rate:20 mL/min Gradient: 30 B to 30 B in 43 min; 220/254 nm; R_{T1}: 27.73; R_{T2}: 40.71.) to provide (1R,2S)-2-(difluoromethyl)-N-(5-(5-methoxybenzo[d]oxazol-2-yl)-8-(methylamino)-2,7-naphthyridin-3-yl)cyclopropane-1-carboxamide (Example 394, the faster peak) as a yellow solid and (1S,2R)-2-(difluoromethyl)-N-(5-(5-methoxybenzo[d]oxazol-2-yl)-8-(methylamino)-2,7-naphthyridin-3-yl)cyclopropane-1-carboxamide (Example 395, the slower peak) as a yellow solid. The two configurations are arbitrarily assigned. LCMS (ESI) m/z 440.1, [M+H]⁺.

HNMR for Example 394:¹H NMR (400 MHz, DMSO-d₆) δ 11.27 (s, 1H), 9.64 (s, 1H), 9.46 (s, 1H), 8.92 (s, 1H), 8.72-8.64 (m, 1H), 7.63 (d, J=8.8 Hz, 7.30 (d, J=2.5 Hz, 1H), 7.01-6.93 (m, 1H), 6.25-5.95 (m, 1H), 3.86 (s, 3H), 3.10 (d, J=4.4 Hz, 3H), 2.47-2.38 (m, 1H), 1.99-1.87 (m, 1H), 1.43-1.29 (m, 2H).

HNMR for Example 395 ¹H NMR (400 MHz, DMSO-d₆) δ 11.27 (s, 1H), 9.64 (s, 1H), 9.46 (s, 1H), 8.92 (s, 1H), 8.72-8.64 (m, 1H), 7.63 (d, J=8.8 Hz, 7.30 (d, J=2.5 Hz, 1H), 7.01-6.93 (m, 1H), 6.25-5.95 (m, 1H), 3.86 (s, 3H), 3.10 (d, J=4.4 Hz, 3H), 2.47-2.38 (m, 1H), 1.99-1.87 (m, 1H), 1.43-1.29 (m, 2H).

Example 396, Example 397, and Example 398: Synthesis of N-(5-(5-methoxybenzo[d]oxazol-2-yl)-8-(methylamino)-2,7-naphthyridin-3-yl)-2-(trifluoromethyl)cyclopropane-1-carboxamide (cis racemate) (Example 396), (1R,2R)—N-(5-(5-methoxybenzo[d]oxazol-2-yl)-8-(methylamino)-2,7-naphthyridin-3-yl)-2-(trifluoromethyl)cyclopropane-1-carboxamide (Example 397) and (1S,2R)—N-(5-(5-methoxybenzo[d]oxazol-2-yl)-8-(methylamino)-2,7-naphthyridin-3-yl)-2-(trifluoromethyl)cyclopropane-1-carboxamide (Example 398)

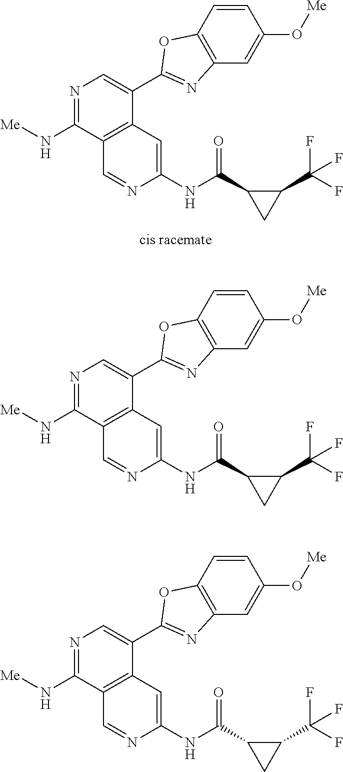 [SMILES: CNc1ncc(-c2nc3cc(OC)ccc3o2)c2cc(NC(=O)[C@@H]3C[C@@H]3C(F)(F)F)ncc12 ; CNc1ncc(-c2nc3cc(OC)ccc3o2)c2cc(NC(=O)[C@@H]3C[C@@H]3C(F)(F)F)ncc12 ; CNc1ncc(-c2nc3cc(OC)ccc3o2)c2cc(NC(=O)[C@H]3C[C@H]3C(F)(F)F)ncc12]

### Step 1: N-(5-(5-methoxybenzo(dioxazol-2-yl)-8-(methylamino)-2,7-naphthyridin-3-yl)-2-(trifluoromethyl)cyclopropane-1-carboxamide (cis racemate)

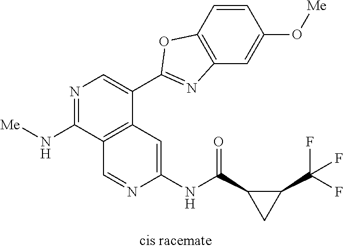 [SMILES: CNc1ncc(-c2nc3cc(OC)ccc3o2)c2cc(NC(=O)[C@@H]3C[C@@H]3C(F)(F)F)ncc12]

N-(5-(5-methoxybenzo[d]oxazol-2-yl)-8-(methylamino)-2,7-naphthyridin-3-yl)-2-(trifluoromethyl)cyclopropane-1-carboxamide (cis racemate) was synthesized using a similar procedure that was previously described in Example 381 by using 4-(5-methoxybenzo[d]oxazol-2-yl)-N¹-methyl-2,7-naphthyridine-1,6-diamine and 2-(trifluoromethyl)cyclopropane-1-carboxylic acid (cis racemate) as the starting material. LCMS (ESI) m/z 458.1, [M+H]⁺.

### Step 2: (1R,2S)—N-(5-(5-methoxybenzoxazol-2-yl)-8-(methylamino)-2,7-naphthyridin-3-yl)-2-(trifluoromethyl)cyclopropane-1-carboxamide and (1S,2R)—N-(5-(5-methoxybenzo[d]oxazol-2-yl)-8-(methylamino)-2,7-naphthyridin-3-yl)-2-(trifluoromethyl)cyclopropane-1-carboxamide

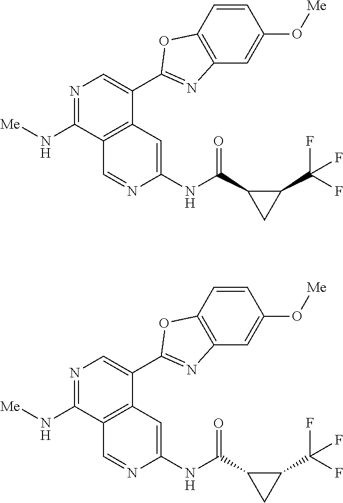 [SMILES: CNc1ncc(-c2nc3cc(OC)ccc3o2)c2cc(NC(=O)[C@@H]3C[C@@H]3C(F)(F)F)ncc12 ; CNc1ncc(-c2nc3cc(OC)ccc3o2)c2cc(NC(=O)[C@H]3C[C@H]3C(F)(F)F)ncc12]

N-(5-(5-methoxybenzo[d]oxazol-2-yl)-8-(methylamino)-2,7-naphthyridin-3-yl)-2-(trifluoromethyl)cyclopropane-1-carboxamide (cis racemate) was separated by Prep-Chiral-HPLC (Column: CHIRAL ART Amylose-SA, 2×25 cm, 5 um; Mobile Phase A: Hex (0.5% 2 M NH₃·MeOH), Mobile Phase B: EtOH:DCM=1:1; Flow rate:20 mL/min; Gradient: 30 B to 30 B in 26 min; 220/254 nm; R_{T1}: 15.163 min; R_{T2}: 23.627 min.) to provide (1R,2S)—N-(5-(5-methoxybenzo[d]oxazol-2-yl)-8-(methylamino)-2,7-naphthyridin-3-yl)-2-(trifluoromethyl)cyclopropane-1-carboxamide (Example 397, the faster peak) and (1S,2R)—N-(5-(5-methoxybenzo[d]oxazol-2-yl)-8-(methylamino)-2,7-naphthyridin-3-yl)-2-(trifluoromethyl)cyclopropane-1-carboxamide (Example 398, the slower peak) as yellow solids. The two configurations are arbitrarily assigned. LCMS (ESI) m/z 458.1, [M+H]⁺.

HNMR for Example 397, NMR (400 MHz, DMSO-d₆) δ 11.18 (s, 1H), 9.61 (s, 1H), 9.45 (s, 1H), 8.91 (s, 1H), 8.71-8.62 (m, 1H), 7.62 (d, J=8.8 Hz, 1H), 7.28 (d, J=2.4 Hz, 1H), 7.01-6.94 (m, 1H), 3.86 (s, 3H), 3.09 (d, J=4.4 Hz, 3H), 2.48-2.42 (m, 1H) 2.33-2.20 (m, 1H), 1.66-1.57 (m, 1H), 1.41-1.31 (m, 1H).

HNMR for Example 398, ¹H NMR (400 MHz, DMSO-d₆) δ11.18 (s, 1H), 9.61 (s, 1H), 9.45 (s, 1H), 8.91 (s, 1H), 8.71-8.62 (m, 1H), 7.62 (d, J=8.8 Hz, 1H), 7.28 (d, J=2.4 Hz, 1H), 7.01-6.94 (m, 1H), 3.86 (s, 3H), 3.09 (d, J=4.4 Hz, 3H), 2.48-2.42 (m, 1H), 2.33-2.20 (m, 1H), 1.66-1.57 (m, 1H), 1.41-1.31 (m, 1H).

### Example 399, Example 400, and Example 401: Synthesis of 2-(fluoromethyl)-N-(5-(5-methoxybenzo[d]oxazol-2-yl)-8-(methylamino)-2,7-naphthyridin-3-yl)cyclopropane-1-carboxamide (cis racemate) (Example 399), (1R,2S)-2-(fluoromethyl)-N-(5-(5-methoxybenzo[d]oxazol-2-yl)-8-(methylamino)-2,7-naphthyridin-3-yl)cyclopropane-16 carboxamide (Example 400) and (1S,2R)-2-(fluoromethyl)-N-(5-(5-methoxybenzo[d]oxazol-2-yl)-8-(methylamino)-2,7-naphthyridin-3-yl)cyclopropane-1-carboxamide (Example 401)

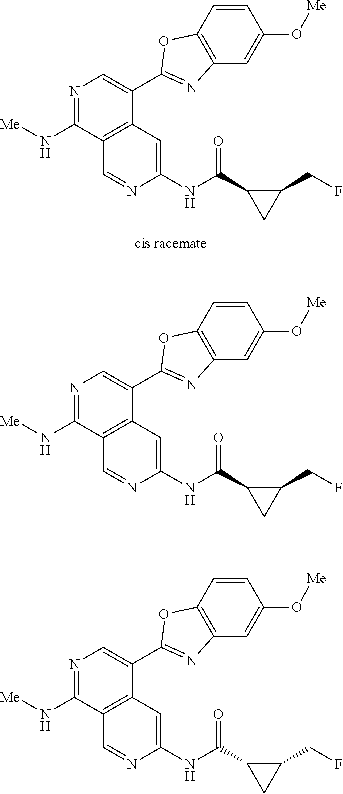 [SMILES: CNc1ncc(-c2nc3cc(OC)ccc3o2)c2cc(NC(=O)[C@@H]3C[C@@H]3CF)ncc12 ; CNc1ncc(-c2nc3cc(OC)ccc3o2)c2cc(NC(=O)[C@@H]3C[C@@H]3CF)ncc12 ; CNc1ncc(-c2nc3cc(OC)ccc3o2)c2cc(NC(=O)[C@H]3C[C@H]3CF)ncc12]

### Step 1: 2-(fluoromethyl)-N-(5-(5-methoxybenzo[d]oxazol-2-yl)-8-(methylamino)-2,7-naphth cyclopropane-1-carboxamide (cis racemate)

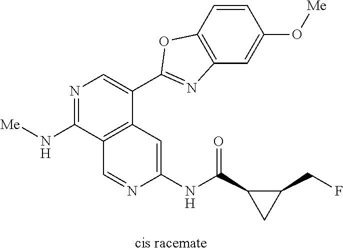 [SMILES: CNc1ncc(-c2nc3cc(OC)ccc3o2)c2cc(NC(=O)[C@@H]3C[C@@H]3CF)ncc12]

2-(fluoromethyl)-N-(5-(5-methoxybenzo[d]oxazol-2-yl)-8-(methylamino)-2,7-naphthyridin-3-yl)cyclopropane-1-carboxamide (cis racemate) was synthesized using a similar procedure that was previously described in Example 381, by using 4-(5-methoxybenzo[d]oxazol-2-yl)-N¹-methyl-2,7-naphthyridine-1,6-diamine and 2-(fluoromethyl)cyclopropane-1-carboxylic acid (cis racemate) as the starting material. LCMS (ESI) m/z 422.2, [M+H]⁺. ¹H NMR (400 MHz, DMSO-d₆) δ 11.10 (s, 1H), 9.63 (s, 1H), 9.45 (s, 1H), 8.91 (s, 1H), 8.72-8.60 (m, 1H), 7.63 (d, J=8.8 Hz, 1H), 7.30 (d, J=2.4 Hz, 1H), 6.97 (dd, J=8.8, 2.4 Hz, 1H), 4.88-4.68 (m, 1H), 4.67-4.45 (m, 1H), 3.86 (s, 3H), 3.09 (d, J=4.6 Hz, 3H), 2.41-2.27 (m, 1H), 1.86-1.72 (m, 1H), 1.23-1.17 (m, 1H), 1.16-1.09 (m, 1H).

### Step 2: (1R,2S)-2-(fluoromethyl)-N-(5-(5-methoxybenzo[d]oxazol-2-yl)-8-(methylamino)-2,7-naphthyridin-3-yl)cyclopropane-1-carboxamide and (1S,2R)-2-(fluoromethyl)-N-(5-(5-methoxybenzo[d]oxazol-2-yl)-8-(methylamino)-2,7-naphthyridin-3-yl)cyclopropane-1-carboxamide

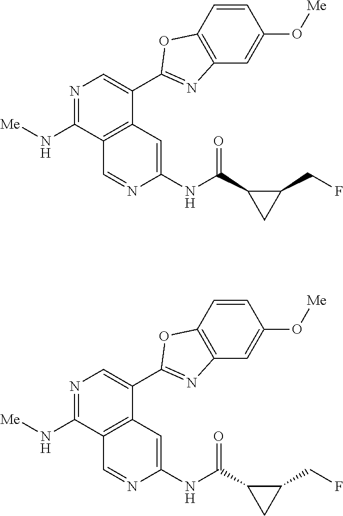 [SMILES: CNc1ncc(-c2nc3cc(OC)ccc3o2)c2cc(NC(=O)[C@@H]3C[C@@H]3CF)ncc12 ; CNc1ncc(-c2nc3cc(OC)ccc3o2)c2cc(NC(=O)[C@H]3C[C@H]3CF)ncc12]

2-(fluoromethyl)-N-(5-(5-methoxybenzo[d]oxazol-2-yl)-8-(methylamino)-2,7-naphthyridin-3-yl)cyclopropane-1-carboxamide (cis racemate) was separated by Prep-Chiral-HPLC (Column: CHIRALPAK IG, 2×25 cm, 5 um; Mobile Phase A: Hex (0.5% 2 M NH₃·MeOH), Mobile Phase B: EtOH:DCM:=1:1; Flow rate:20 mL/min; (=gradient: 30% B to 30% B in 20 min; 220/254 nm; R_{T1}: 14.69 min; R_{T2}: 17.15 min.) to provide (1R,2S)-2-(fluoromethyl)-N-(5-(5-methoxybenzo[d]oxazol-2-yl)-8-(methylamino)-2,7-naphthyridin-3-yl)cyclopropane-1-carboxamide (Example 400, the faster peak) and (1S,2R)-2-(fluoromethyl)-N-(5-(5-methoxybenzo[d]oxazol-2-yl)-8-(methylamino)-2,7-naphthyridin-3-yl)cyclopropane-1-carboxamide (Example 401, the slower peak) as yellow solids. The two configurations are arbitrarily assigned. LCMS (ESI) m/z 422.2, [M+H]⁺.

HNMR for Example 400: ¹H NMR. (400 MHz, DMSO-d₆) δ 11.10 (s, 1H), 9.63 (s, 1H), 9.45 (s, 1H), 8.91 (s, 1H), 8.72-8.60 (m, 1H), 7.63 (d, J=8.8 Hz, 1H), 7.30 (d, J=2.4 Hz, 1H), 6.97 (dd, 8.8, 2.4 Hz, 1H), 4.88-4.68 (m, 1H), 4.67-4.45 (m, 1H), 3.86 (s, 3H), 3.09 (d, J=4.6 Hz, 3H), 2.41-2.27 (m, 1H), 1.86-1.72 (m, 1H), 1.23-1.17 (m, 1H), 1.16-1.09 (m, 1H).

HNMR for Example 401: ¹H NMR (400 MHz, DMSO-d₆) δ 11.10 (s, 1H), 9.63 (s, 1H), 9.45 (s, 1H), 8.91 (s, 1H), 8.72-8.60 (m, 1H), 7.63 (d, J=8.8 Hz, 1H), 7.30 (d, J=2.4 Hz, 1H), 6.97 (dd, J=8.8, 2.4 Hz, 1H), 4.88-4.68 (m, 1H), 4.67-4.45 (m, 1H), 3.86 (s, 3H), 3.09 (d, J=4.6 Hz, 3H), 2.41-2.27 (m, 1H), 1.86-1.72 (m, 1H), 1.23-1.17 (m, 1H), 1.16-1.09 (m, 1H).

### Example 402: Synthesis of 3,4-dihydroxy-N-(5-(5-methoxybenzo[d]oxazol-2-yl)-8-(methylamino)-2,7-naphthyridin-3-yl)-3-methylbutanamide

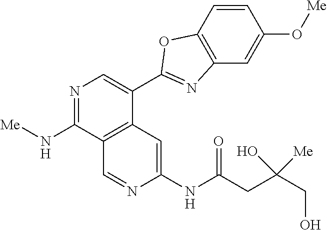 [SMILES: CNc1ncc(-c2nc3cc(OC)ccc3o2)c2cc(NC(=O)CC(C)(O)CO)ncc12]

### Step 1: N-(5-(5-methoxybenzo[d]oxazol-2-yl)-8-(methylamino methylbut-3-enamide

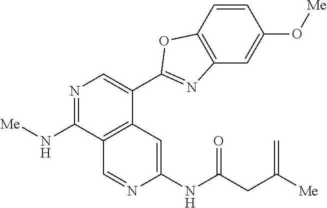 [SMILES: C=C(C)CC(=O)Nc1cc2c(-c3nc4cc(OC)ccc4o3)cnc(NC)c2cn1]

To a solution of 4-(5-methoxybenzo[d]oxazol-2-yl)-N¹-methyl-2,7-naphthyridine-1,6-diamine (50.0 mg; 0.156 mmol; 1.00 eq.) in pyridine (4 mL; 0.006 mmol, 0.20 eq.) was added 3-methylbut-3-enoyl chloride (18.4 mg; 0.155 mmol; 1.00 eq.) at 0° C. The reaction was stirred at room temperature for 1 hour under nitrogen atmosphere. The desired product was detected via LCMS. The reaction was diluted with EtOAc (60 and washed with brine (3×4 The organic phase was dried with Na₂SO₄, filtered, and concentrated under reduced pressure. The residue was purified by flash chromatography on silica gel column using 2-10% of MeOH in CH₂Cl₂ as eluent to provide N-(5-(5-methoxybenzo[d]oxazol-2-yl)-8-(methylamino)-2,7-naphthyridin-3-yl)-3-methylbut-3-enamide as a yellow solid (54.0 mg, 85.8%), LCMS (ESI) m/z 404.2, [M+H]⁺.

### Step 2: 3,4-dihydroxy-N-(5-(5-methoxybenzo[d]oxazol-2-yl)-8-(methylamino)-2,7-naphthyridin-3-yl)-3-methylbutanamide

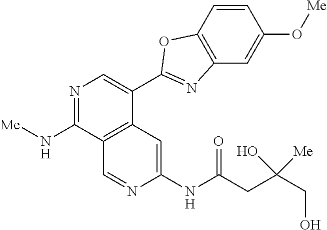 [SMILES: CNc1ncc(-c2nc3cc(OC)ccc3o2)c2cc(NC(=O)CC(C)(O)CO)ncc12]

To a solution of N-(5-(5-methoxybenzo[d]oxazol-2-yl)-8-(methylamino)-2,7-naphthyridin-3-yl)-3-methylbut-3-enamide (40.0 Trig; 0.099 mmol; 1.00 eq.) in a mixture solvent of acetone/water (4:1, 3 mL) was added K₂OsO₂(OH)₄ (2.2 mg; 0.006 mmol; 0.06 eq.) and 4-methylmorpholin-4-ium-4-olate (23.2 mg; 0.198 mmol; 2.00 eq.) at room temperature. The reaction was stirred for 36 hours at room temperature under nitrogen atmosphere. The desired product was detected via LCMS. The resulting mixture was concentrated under vacuum. The residue was purified by flash chromatography on silica gel column using 2-10% of MeOH in CH₂Cl₂ as eluent to provide the crude product. The crude product was purified by flash chromatography on pre-packed C18 column using 20-70% of MeCN in water (10 mmol/L NH₄HCO₃) to provide 3,4-dihydroxy-N-(5-(5-methoxybenzo[d]oxazol-2-yl)-8-(methylamino)-2,7-naphthyridin-3-yl)-3-methylbutanamide as a yellow solid (4.1 mg, 9.4%). LCMS (ESI) m/z 438.2, [M+H]⁺.

### Example 403: Synthesis of 5-(((5-(5-methoxybenzo[d]oxazol-naphthyridin-3-yl)amino)dihydrofuran-2(3H)-one

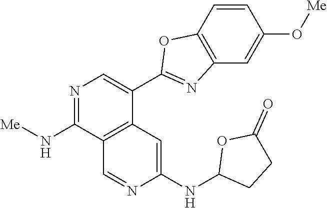 [SMILES: CNc1ncc(-c2nc3cc(OC)ccc3o2)c2cc(NC3CCC(=O)O3)ncc12]

5-((5-(5-methoxybenzo[d]oxazol-2-yl)-8-(methylamino)-2,7-naphthyridin-3-yl)amino)dihydrofuran-2(3H)-one was synthesized using a similar procedure that was previously described in Example 381, by using 4-(5-methoxybenzo[d]oxazol-2-yl)-N¹-methyl-2,7-naphthyridine-1,6-diamine and 2-methoxycyclopropane-1-carboxylic acid as the starting material. LCMS (ESI) m/z 406.1, [M+H]⁺. ¹H NMR (400 MHz, DMSO-d₆) δ 9.74 (s, 1H), 9.50 (s, 1H), 8.94 (s, 1H), 8.80-8.71 (m, 1H), 7.64 (d, J=8.8 Hz, 1H), 7.29 (d, J=2.4 Hz, 1H), 7.01-6.93 (m, 1H), 6.37-6.33 (m, 1H), 6.23 (d, J=6.4 Hz, 1H), 3.86 (s, 3H), 3.11 (d, J=4.4 Hz, 3H), 2.94-2.80 (m, 1H), 2.55-2.50 (m, 1H), 2.45-2.32 (m, 1H), 1.98-1.87 (m, 1H).

### Example 404: Synthesis of 1-hydroxy-N-(5-(5-methoxybenzo[d]oxazol-2-yl)-8-(methylamino)-2,7-naphthyridin-3-yl)cyclopropane-1-carboxamide

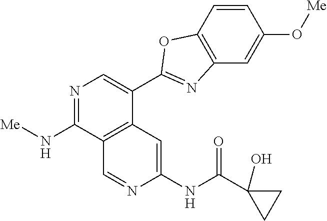 [SMILES: CNc1ncc(-c2nc3cc(OC)ccc3o2)c2cc(NC(=O)C3(O)CC3)ncc12]

### Step 1: methyl 1-(benzyloxy)-cyclopropane-1-carboxylate

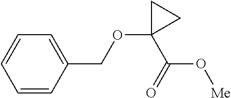 [SMILES: COC(=O)C1(OCc2ccccc2)CC1]

To a stirred solution of methyl 1-hydroxycyclopropane-1-carboxylate (1.00 g; 8.612 mmol; 1.00 eq.) in DMF (10 mL) was added NaH (60%) (0.41 g; 17.224 mmol; 2.00 eq.) at 0° C. The mixture was stirred at 0° C. for 30 minutes under nitrogen atmosphere. To this mixture was added (bromomethyl)benzene (1.77 g; 10.334 mmol, 1.20 eq.). The resulting solution was stirred at room temperature overnight under nitrogen atmosphere. The reaction was quenched with saturated NH₄Cl aqueous solution (5 mL) and extracted with EtOAc (3×50 mL). The combined organic lavers were washed with brine (3×50 mL), dried over anhydrous Na₂SO₄, After filtration, the filtrate was concentrated under reduced pressure. The residue was purified by flash chromatography on silica gel column using 0-20% of EtOAc in petroleum ether as eluent to provide methyl 1-(benzyloxy)cyclopropane-1-carboxylate as a white solid (300.0 mg, 16.8%). ¹H NMR (400 MHz, DMSO-d₆) δ 7.39-7.26 (m, 5H), 4.60 (s, 2H), 3.69 (s, 3H), 1.31-1.20 (m, 4H).

### Step 2: 1-(benzyloxy)cyclopropane-1-carboxylic acid

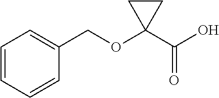 [SMILES: O=C(O)C1(OCc2ccccc2)CC1]

To a solution of methyl 1-(benzyloxy)cyclopropane-1-carboxylate (300.0 mg; 1.455 mmol; 1.00 eq.) in a mixture solvent of THF/MeOH (1:1, 5 mL) was added a solution of NaOH (174.5 mg; 4.363 mmol; 3.00 eq.) in water (2.2 mL). The resulting solution was stirred at room temperature for 2 hours. The solvent was concentrated under vacuum. The residue was purified by flash chromatography on pre-packed C18 column using 10-70% of MeCN/THF (3:1) in water (10 mmol/L NH₄HCO₃) as eluent to provide 1-(benzyloxy)cyclopropane-1-carboxylic acid as a white solid (200.0 mg, 71.5%).

### Step 3: 1-(benzyloxy)-N-(5-(5-methoxybenzo[d]oxazol-2-yl)-8-(methylamino)-2,7-naphthyridin-3-yl)cyclopropane-1-carboxamide

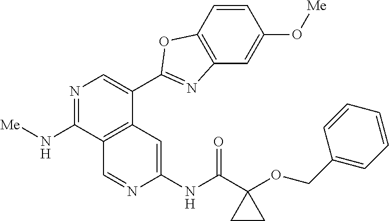 [SMILES: CNc1ncc(-c2nc3cc(OC)ccc3o2)c2cc(NC(=O)C3(OCc4ccccc4)CC3)ncc12]

To a stirred mixture of 4-(5-methoxybenzo[d]oxazol-2-yl)-N¹-methyl-2,7-naphthyridine-1,6-diamine (150.0 mg; 0.480 mmol; 1.00 eq.) and 1-(benzyloxy)cyclopropane-1-carboxylic acid (83.1 mg; 0.432 mmol; 0.90 eq.) in pyridine (10 mL) was added POCl₃ (220.8 mg; 1.440 mmol; 3.00 eq.) dropwise at 0° C. The resulting mixture was stirred at room temperature for 30 min. The reaction was quenched with water (10 mL) and extracted with EtOAc (3×50 mL). The combined organic layers were washed with brine (3×10 mL), dried over anhydrous-Na₂SO₄. After filtration, the filtrate was concentrated under reduced pressure. The residue was purified by flash chromatography on silica gel column using 5-25% of MeOH in CH₂Cl₂ to afford 1-(benzyloxy)-N-(5-(5-methoxybenzo[d]oxazol-2-yl)-8-(methylamino)-2,7-naphthyridin-3-yl)cyclopropane-1-carboxamide as a yellow solid (100.0 mg, 38.7%). LCMS (ESI) m/z 496.1, [M+H]⁺.

### Step 4: 1-hydroxy-N-(5-(5-methoxybenzo[d]oxazol-2-yl)-8-(methylamino)-2,7-naphthyridin-3-yl)cyclopropane-1-carboxamide

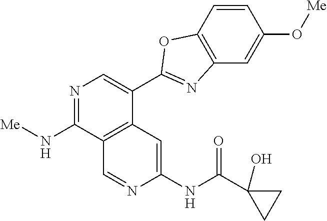 [SMILES: CNc1ncc(-c2nc3cc(OC)ccc3o2)c2cc(NC(=O)C3(O)CC3)ncc12]

To a solution of 1-(benzyloxy)-N-(5-(5-methoxybenzo[d]oxazol-2-yl)-8-(methylamino)-2,7-naphthyridin-3-O cyclopropane-1-carboxamide (100.0 mg; 0.202 mmol; 1.00 eq.) in THF (20 mL) was added 10% Pd/C (100.0 mg; 100% w/w) under nitrogen atmosphere. The resulting solution was stirred at room temperature for 36 hours under hydrogen atmosphere (2 atm). The desired product was detected via LCMS. After filtration, the filtrate was concentrated under reduced pressure. The residue was purified by flash chromatography on pre-packed C18 column using 10-70% of MeCN/THF (3:1) in water (10 mmol/L NH₄HCO₃) as eluent to provide 1-hydroxy-N-(5-(5-methoxybenzo[d]oxazol-2-yl)-8-(methylamino)-2,7-naphthyridin-3-yl)cyclopropane-1-carboxamide as a yellow solid (26.3 mg, 31.2%). LCMS (ESI) m/z 406.1, [M+H]⁺. NMR (400 MHz, DMSO-d₆) δ 9.69 (s, 1H), 9.66 (s, 1H), 9.45 (s, 1H), 8.93 (5, 1H), 8.78-8.66 (m, 1H), 7.63 (d, J=8.8 Hz, 1H), 7.28 (d, J=2.4 Hz, 1H), 7.04-6.94 (m, 1H), 6.92 (5, 1H), 3.85 (s, 3H), 3.09 (d, J=4.4 Hz, 3H), 1.37-1.24 (m, 2H), 1.18-1.05 (m, 2H).

### Example 405: Synthesis of 2-(2-hydroxypropan-2-yl)-N-(5-(5-methoxybenzo[d]oxazol-2-yl)-8-(methylamino)-2,7-naphthyridin-3-yl)cyclopropane-1-carboxamide (cis racemate)

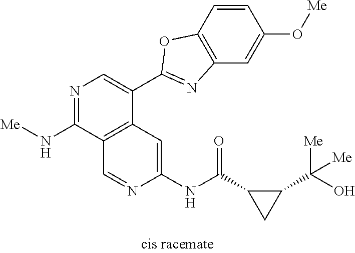 [SMILES: CNc1ncc(-c2nc3cc(OC)ccc3o2)c2cc(NC(=O)[C@H]3C[C@H]3C(C)(C)O)ncc12]

To a stirred solution of ethyl 2-((5-(5-methoxybenzo[d]oxazol-2-yl)-8-(methylamino)-2,7-naphthyridin-3-yl)carbamoyl)cyclopropane-1-carboxylate (ci s racemate, Example 391) (40.0 mg; 0.087 mmol; 1.00 eq.) in THF (4 mL) was added methylmagnesium bromide (1 M in THF; 0.3 mL) drop-wise at 0° C. under nitrogen atmosphere. The resulting mixture was stirred at 0° C. overnight. The reaction was quenched by the addition of water (2 mL), extracted with CH₂Cl₂ (3×10 mL). The combined organic layers were washed with brine (2×10 mL), dried over anhydrous Na₂SO₄. After filtration, the filtrate was concentrated under reduced pressure. The residue was purified by flash chromatography on silica gel column using 0-1.0% of MeOH in CH₂Cl₂ as eluent to provide 2-(2-hydroxypropan-2-yl)-N-(5-(5-methoxybenzo[d]oxazol-2-yl)-8-(methylamino)-2,7-naphthyridin-3-yl)cyclopropane-1-carboxamide (cis racemate) as a yellow solid (4.4 mg, 10.4%). LCMS (ESI) m/z 448.2, [M+H]⁺. ¹H NMR. (400 MHz, DMSO-d₆) δ 11.29 (s, 1H), 9.64 (s, 1H), 9.45 (s, 1H), 8.92 (s, 1H), 8.71-8.48 (m, 1H), 7.61 (d, J=8.8 Hz, 1H), 7.23 (d, J=2.4 Hz, 1H), 7.04-6.91 (m, 1H), 5.24 (s, 1H), 3.85 (s, 3H), 3.10 (d, J=4.4 Hz, 3H), 2.08-2.01 (m, 1H), 1.57-1.49 (m, 1H), 1.48-1.42 (m, 1H), 1.26 (s, 3H), 1.24 (s, 3H), 1.20-1.14 (m, 1H).

### Example 406: Synthesis of 2-(2-hydroxypropan-2-yl)-N-(5-(5-methoxybenzo[d]oxazol-2-yl)-8-(methylamino)-2,7-naphthyridin-3-yl)cyclopropane-1-carboxamide (trans racemate)

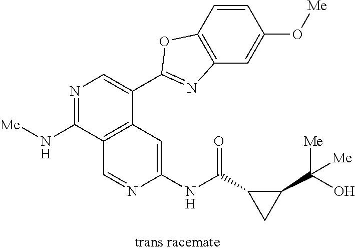 [SMILES: CNc1ncc(-c2nc3cc(OC)ccc3o2)c2cc(NC(=O)[C@H]3C[C@@H]3C(C)(C)O)ncc12]

To a stirred solution of ethyl 2-(5-(5-methoxybenzo[d]oxazol-2-yl)-8-(methylamino-2,7-naphthyridin-3-yl)carbamoyl)cyclopropane-1-carboxylate (trans racemate, Example 392) (75.0 mg; 0.163 mmol; 1.00 eq.) in THF (6 mL) was added methylmagnesium bromide (1 Min THF; 8.6 mL; 53.00 eq.) drop-wise at 0° C. under nitrogen atmosphere. The resulting mixture was stirred at room temperature overnight. The reaction mixture was quenched by the addition of water (1 mL), extracted with EtOAc (3×10 mL). The combined organic layers were washed with brine (2×10 mL), dried over anhydrous Na₂SO₄. After filtration, the filtrate was concentrated under reduced pressure. The residue was purified by flash chromatography on silica gel column using 0-10% of MeOH in CH₂Cl₂ as eluent to provide 2-(2-hydroxypropan-2-yl)-N-(5-(5-methoxybenzo[d]oxazol-2-yl)-8-(methylamino)-2,7-naphthyridin-3-yl)cyclopropane-1-carboxamide (trans racemate) as a yellow solid (26.2 mg, 35.7%). LCMS (ESI) m/z 448.2, [M+H]⁺. ¹H NMR (400 MHz, DMSO-d₆) δ 10.91 (s, 1H), 9.66 (s, 1H), 9.43 (s, 1H), 8.89 (s, 1H), 8.79-8.26 (m, 1H), 7.63 (d, J=8.4 Hz, 1H), 7.29 (d, J=2.4 Hz, 1H), 7.07-6.71 (m, 1H), 4.27 (s, 1H), 3.86 (s, 3H), 3.10 (d, J=4.4 Hz, 3H), 2.19-1.97 (m, 1H), 1.53-1.40 (m, 1H), 1.18 (s, 6H), 1.02-0.91 (m, 2H).

### Example 407: Synthesis of (1S,2,S)-2-fluoro-N-(8-(methylamino)-5-(5-((S)-2-methylmorpholino)benzo[d]oxazol-2-yl)-2,7-naphthyridin-3-yl)cyclopropane-1-carboxamide

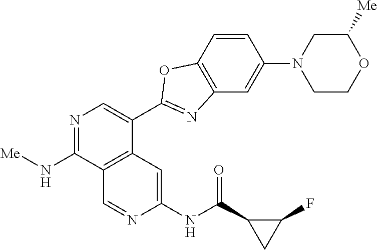 [SMILES: CNc1ncc(-c2nc3cc(N4CCO[C@@H](C)C4)ccc3o2)c2cc(NC(=O)[C@@H]3C[C@@H]3F)ncc12]

### Step 1: (S)—N¹-methyl-4-(5-(2-methylmorpholino)benzo[d]oxazol-2-yl)-2,7-naphthyridine-1-6-diamine

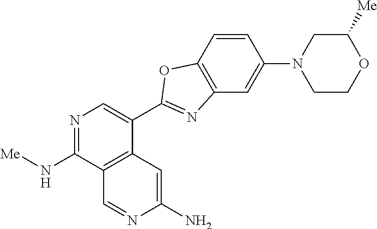 [SMILES: CNc1ncc(-c2nc3cc(N4CCO[C@@H](C)C4)ccc3o2)c2cc(N)ncc12]

To a solution of (S)—N-(8-(methylamino)-5-(5-(2-methylmorpholino)benzo[d]oxazol-2-yl)-2,7-naphthyridin-3-yl)cyclopropanecarboxamide (Example 289) (1.20 g; 2.620 mmol; 1.00 eq.) in a mixture solvent of DMSO/MeOH (1:5, 60 mL) was added a solution of NaOH (1.05 g; 26.200 mmol; 10.00 eq.) in water (7 mL). The reaction was stirred at 60° C. for 18 hours. The mixture was concentrated under vacuum to remove MeOH, to this mixture was added H₂O (200 mL) to precipitate solids. The solids were collected by filtration and washed with H₂O (3×20 mL) to provide (S)—N¹-methyl-4-(5-(2-methylmorpholino)benzo[d]oxazol-2-yl)-2,7-naphthyridine-1,6-diamine as a yellow solid (910.0 mg, 88.2%). LCMS (ER) m/z 391.2, [M+H]⁺.

### Step 2: (1S,2S)-2-fluoro-N-(8-(methylamino)-5-(5-((S)-2-methylmorpholino)benzo[d]oxazol-2-yl)-2,7-naphthyridin-3-yl)cyclopropane-1-carboxamide

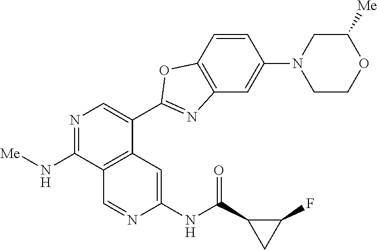 [SMILES: CNc1ncc(-c2nc3cc(N4CCO[C@@H](C)C4)ccc3o2)c2cc(NC(=O)[C@@H]3C[C@@H]3F)ncc12]

To a solution of (S)-M-methyl-4-(5-(2-methyl morpholino)benzo[d]oxazol-2-yl)-2,7-naphthyridine-1,6-diamine (80.0 mg; 0.205 mmol; 1.00 eq.) and (1S,2S)-2-fluorocyclopropane-1-carboxylic acid (19.1 mg; 0.184 mmol; 0.90 eq.) in pyridine (5 mL) was added POCl₃ (114.5 mg; 0.747 mmol; 3.00 eq.) dropwise at 0° C. The resulting mixture was stirred at room temperature for 2 hours. The solution was quenched by the addition of water (10 mL), extracted with EtOAc (3×50 mL). The combined organic layers were washed with brine (3×10 mL), dried over anhydrous Na₂SO₄. After filtration, the filtrate was concentrated under reduced pressure. The residue was purified by flash chromatography on silica gel column using 5-10% of MeOH in CH₂Cl₂ as eluent to afford a crude product. The crude product was purified by flash chromatography on pre-packed C18 column using 30-60% of MeOH in water (10 mmol/L NH₄HCO₃) as eluent to provide (1S,2S)-2-fluoro-N-(8-(methylamino)-5-(5-((S)-2-methylmorpholino)benzo[d]oxazol-2-yl)-2,7-naphthyridin-3-yl)cyclopropane-1-carboxamide as a yellow solid (30.5 mg, 30.3%). LCMS (ESI) m/z 477.2, [M+H]⁺. ¹H NMR (400 MHz, DMSO-d₆) δ 11.08 (s, 1H), 9.66 (s, 1H), 9.45 (s, 1H), 8.90 (s, 1H), 8.70-8.60 (m, 1H), 7.59 (d, J==9.2 HZ, 1H), 7.24 (d, J=2.4 Hz, 1H), 7.10-7.01 (m, 1H), 5.10-4.87 (m, 1H), 3.97-3.91 (m, 1H), 3.76-3.60 (m, 3H), 3.56-3.51 (m, 1H), 3.09 (d, J=4.4 Hz, 3H), 2.77-2.68 (m, 1H), 2.44-2.36 (m, 1H), 2.34-2.28 (m, 1H), 1.80-1.69 (m, 1H), 1.28-1.17 (m, 4H).

### Example 408 Synthesis of (1S,2R)-2-methyl-N-(8-(methylamino)-5-(5-((S)-2-methylmorpholino)benzo[d]oxazol-2-yl)-2,7-naphthyridin-3-yl)cyclopropane-1-carboxamide

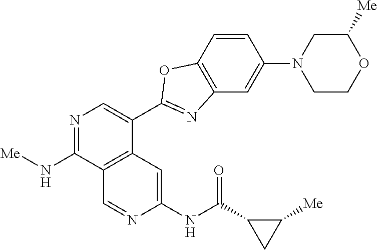 [SMILES: CNc1ncc(-c2nc3cc(N4CCO[C@@H](C)C4)ccc3o2)c2cc(NC(=O)[C@H]3C[C@H]3C)ncc12]

(1S,2R)-2-methyl-N-(8-(methylamino)-5-(5-((S)-2-methylmorpholino)benzo[d]oxazol-2-yl)-2,7-naphthyridin-3-yl)cyclopropane-1-carboxamide was synthesized using a similar procedure that was previously described in Example 407, by using (S)—N¹-methyl-4-(5-(2-methylmorpholino)benzo[d]oxazol-2-yl)-2,7-naphthyridine-1,6-diamine and (1S,2R)-2-methylcyclopropane-1-carboxylic acid as the starting material. LCMS (ESI) 473.2, [M+H]⁺. ¹H NMR (400 MHz, DMSO-d₆) δ 10.91 (s, 1H), 9.60 (s, 1H), 9.43 (s, 1H), 8.88 (s, 1H), 8.67-8.59 (m, 1.14), 7.58 (d, J=8.8 Hz, 1H), 7.24 (d, J=2.4 Hz, 1H), 7.11-7.02 (m, 1H), 3.98-3.89 (m, 1H), 3.75-3.59 (m, 3H), 3.56-3.52 (m, 1H), 3.09 (d, J=4.4 Hz, 3H), 2.76-2.68 (m, 1H), 2.44-2.36 (m, 1H), 2.17-2.08 (m, 1H), 1.38-1.28 (m, 1H), 1.21-1.14 (m, 6H), 1.07-0.98 (m, 1H), 0.94-0.86 (m, 1H).

### Example 409: Synthesis of (S)-1-methyl-N-(8-(methylamino)-5-(5-(2-methylmorpholino)benzo[d]oxazol-2-yl)-2,7-naphthyridin-3-yl)cyclopropane-4-carboxamide

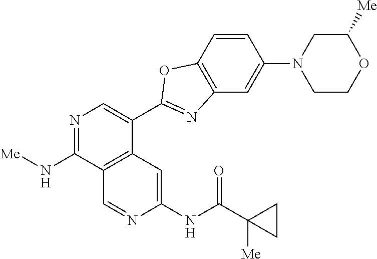 [SMILES: CNc1ncc(-c2nc3cc(N4CCO[C@@H](C)C4)ccc3o2)c2cc(NC(=O)C3(C)CC3)ncc12]

(S)-1-methy N-(8-(methylamino)-5-(5-(2-methylmorpholino)benzo[d]oxazol-2-yl)-2,7-naphthyridin-3-yl)cyclopropane-1-carboxamide was synthesized using a similar procedure that was previously described in Example 407, by using (S)—N¹-methyl-4-(5-(2-methylmorpholino)benzo[d]oxazol-210-2,7-naphthyridine-1,6-diamine and 1-methylcyclopropane-1-carboxylic acid as the starting material. LCMS (ESI) m/z 473.2, [M+H]⁺. ¹H NMR (400 MHz, DMSO-d₆) δ 9.74 (s, 1H), 9.61 (s, 1H), 9.46 (s, 1H), 8.90 (s, 1H), 8.71-8.61 (m, 1.14), 7.59 (d, J=8.8 Hz, 1H), 7.25 (d, J=2.0 Hz, 1H), 7.10-7.03 (m, 1H), 3.98-3.90 (m, 1H), 3.74-3.60 (m, 3H), 3.56-3.50 (m, 1H), 3.09 (d, J=4.4 Hz, 3H), 2.77-2.67 (m, 1H), 2.44-2.355 (m, 1H), 1.49 (s, 3H), 1.27-1.18 (m, 5H), 0.75-0.69 (m, 2H).

### Examples 410, 411, and 412

### Synthesis of 2-(fluoromethyl)-N-(8-(methylamino)-5-(5-((S)-2-methylmorpholino)benzo[d]oxazol-2-yl)-2,7-naphthyridin-3-yl)cyclopropane-1-carboxamide (cis racemate) (Example 410), (1S,2R)-2-(fluoromethyl)-N-(8-(methylamino)-5-(5-((S)-2-methylmorpholino)benzo[d]oxazol-2-yl)-2,7-naphthyridin-3-yl)cyclopropane-1-carboxamide (Example 411) and (1R,2S)-2-(fluoromethyl)-N-(8-(methylamino)-5-(5-((S)-2-methylmorpholino)benzo(dioxazol-2-yl)-2,7-naphthyridin-3-yl)cyclopropane-1-carboxamide (Example 412)

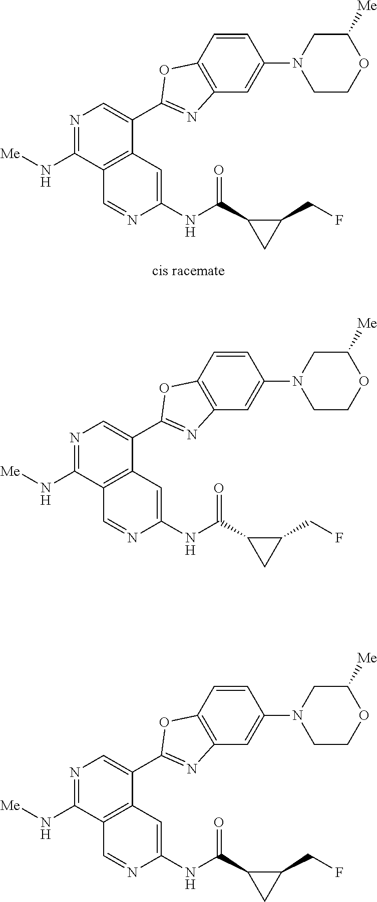 [SMILES: CNc1ncc(-c2nc3cc(N4CCO[C@@H](C)C4)ccc3o2)c2cc(NC(=O)[C@@H]3C[C@@H]3CF)ncc12 ; CNc1ncc(-c2nc3cc(N4CCO[C@@H](C)C4)ccc3o2)c2cc(NC(=O)[C@H]3C[C@H]3CF)ncc12 ; CNc1ncc(-c2nc3cc(N4CCO[C@@H](C)C4)ccc3o2)c2cc(NC(=O)[C@@H]3C[C@@H]3CF)ncc12]

### Step 1: ethyl 2-(hydroxymethyl)cyclopropane-1-carboxylate (cis racemate)

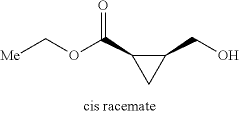

A solution of 2-(ethoxycarbonyl)cyclopropane-1-carboxylic acid (cis racemate) (2.00 g; 12.646 mmol; 1.00 eq.) and THF (0 mL) was cooled to 0° C., to this solution was added BH₃-THF (1 M in THF, 19 mL) drop-wise over 30 minutes under nitrogen atmosphere. The resultant mixture was stirred at room temperature overnight. After the reaction was completed, it was quenched with EtOH (10 mL) in ice/water bath. The mixture was concentrated under reduced pressure. The residue was purified by flash chromatography on silica gel column using EtOAc in petroleum ether (0-50%) as eluent to provide ethyl 2-(hydroxymethyl)cyclopropane-1-carboxylate (cis racemate) as a colorless liquid (2.58 g, crude).

### Step 2: ethyl 2-(fluoromethyl)cyclopropane-1-carboxylate (cis racemate)

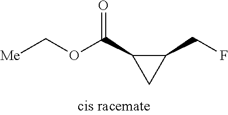

A solution of ethyl 2-(hydroxymethyl)cyclopropane-1-carboxylate (cis racemate) (2.58 g; 17.895 mmol, 1.00 eq.) in CH₂Cl₂ (15 mL) was added to a solution of BAST (3.98 g; 35,791 mmol, 2.00 eq.) in CH₂Cl₂ (15 mL) at 0° C. The resulting solution was stirred at room temperature for 14 hours under nitrogen atmosphere. The mixture was poured into saturated-NaHCO₃ aqueous solution (20 mL) and extracted with CH₂Cl₂ (3×50 mL). The combined organic layers were washed with brine (2×10 mL), dried over anhydrous Na₂SO₄. After filtration, the filtrate was concentrated to afford ethyl 2-(fluoromethyl)cyclopropane-1-carboxylate (cis racemate) as a colorless oil (3.67 g, crude).

### Step 3: 2-(fluoromethyl)cyclopropane-1-carboxylic acid (res racemate)

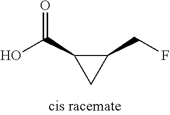

To a solution lithium hydroxide monohydrate (1.05 g; 25.109 mmol; 1.00 eq.) in a mixture solvent of THF/H₂O (1:1, 30 mL) was added ethyl 2-(fluoromethyl)cyclopropane-1-carboxylate (cis racemate) (3.67 g; 25.109 mmol; 1.00 eq.). The resulting mixture was stirred at room temperature for 2 hours. The THF was removed under reduced pressure and the aqueous phase was adjusted to pH=3 with 2 M HCl. The mixture was extracted with CH₂Cl₂ (3×10 mL). The combined organic layers were concentrated. The residue was purified by flash chromatography on silica gel column using 0-50% of EtOAc in petroleum ether as eluent to provide 2-(fluoromethyl)cyclopropane-1-carboxylic acid (cis racemate) as a white solid (173.2 mg, 11.5%).

### Step 4: 2-(fluoromethyl)-N-(8-(methylamino)-5-(5-((S)-2-methylmorpholino)benzo[d]oxazol-2-yl)-2,7-naphthyridin-3-yl)cyclopropane-1-carboxamide (cis racemate)

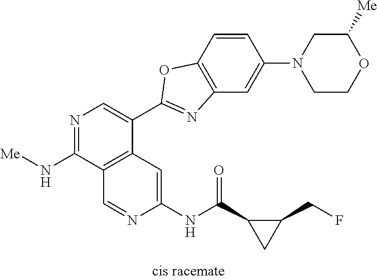 [SMILES: CNc1ncc(-c2nc3cc(N4CCO[C@@H](C)C4)ccc3o2)c2cc(NC(=O)[C@@H]3C[C@@H]3CF)ncc12]

2-(fluoromethyl)-N-(8-(methylamino)-5-(5-((S)-2-methylmorpholino)benzo[d]oxazol-2-yl)-2,7-naphthyridin-3-yl)cyclopropane-1-carboxamide (cis racemate) was synthesized using a similar procedure that was previously described in Example 407, by using (S)—N¹-methyl-4-(5-(2-methylmorpholino)benzo[d]oxazol-2-yl)-2,7-naphthyridine-1,6-diamine and 2-(fluoromethyl)cyclopropane-1-carboxylic acid (cis racemate) as the starting material. LCMS (ESI) m/z [M+H]⁺. ¹H NMR (400 MHz, DMSO-d₆) 11.09 (s, 1H), 9.61 (s, 1H), 9.44 (s, 1H), 8.89 (s, 1H), 8.70-8.57 (m, 1H), 7.58 (d, J=8.8 Hz, 1H), 7.24 (d, J=2.4 Hz, 1H), 7.07 (dd, 8.8, 2.4 Hz, 1H), 4.91-4.67 (m, 1H), 4.67-4.45 (m, 1H), 4.01-3.90 (m, 1H), 3.77-3.66 (m, 2H), 3.66-3.47 (m, 2H), 3.09 (d, J=4.4 Hz, 3H), 2.77-2.66 (m, 1H), 2.44-2.35 (m, 1H), 2.35-2.28 (m, 1H), 1.87-1.71 (m, 1H), 1.26-1.12 (m, SH).

### Step 5: (1S,2R)-2-(fluoromethyl)-N-(8-(methylamino)-5-(5-((S)-2-methylmorpholino)benzo[d]oxazol-2-yl)-2,7-naphthyridin-3-yl)cyclopropane-1-carboxamide and (1R,2S)-2-(fluoromethyl)-N-(8-(methylamino)-5-(5-((S)-2-methylmorpholino)benzo[d]oxazol-2-yl)-2,7-naphthyridin-3-yl)cyclopropane-1-carboxamide

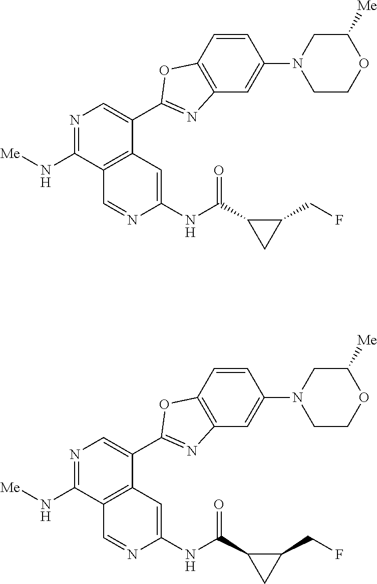 [SMILES: CNc1ncc(-c2nc3cc(N4CCO[C@@H](C)C4)ccc3o2)c2cc(NC(=O)[C@H]3C[C@H]3CF)ncc12 ; CNc1ncc(-c2nc3cc(N4CCO[C@@H](C)C4)ccc3o2)c2cc(NC(=O)[C@@H]3C[C@@H]3CF)ncc12]

2-(fluoromethyl)-N-(8-(methylamino)-5-(5-((S)-2-methylmorpholino)benzo[d]oxazol-2-yl)-2,7-naphthyridin-3-yl)cyclopropane-1-carboxamide (cis racemate) was separated by Prep-Chiral-HPLC (Column: CHIRALPAK IH, 2×25 cm, 5 um; Mobile Phase A: Hex (0.5% 2 M NH₃·MeOH), Mobile Phase B: EtOH:DCM=1:1; Flow rate:20 ml/min, Gradient: 20% B to 20% B in 24 min; 220/254 nm; R_{T1}: 15.66 min; R_{T2}: 21.01 min.) to provide (1S,2R)-2-(fluoromethyl)-N-(8-(methylamino)-5-(5-((,9-2-methylmorpholino)benzo[d]oxazol-2-yl)-2,7-naphthyridin-3-yl)cyclopropane-1-carboxamide (Example 410, the faster peak) and (1R,2S)-2-(fluoromethyl)-N-(8-(methylamino)-5-(5-((S)-2-methylmorpholino)benzo[d]oxazol-2-yl)-2,7-naphthyridin-3-yl)cyclopropane-1-carboxamide (Example 411, the slower peak) as yellow solids. The two configurations are arbitrarily assigned. LCMS (ESI) m/z 491.2, [M+H]⁺.

HNMR for Example 411: ¹H NMR (400 MHz, DMSO-d₆) δ 11.09 (s, 1H), 9.61 (s, 1H), 9.44 (s, 1H), 8.89 (s, 1H), 8.70-8.57 (m, 1H), 7.58 (d, J=8.8 Hz, 1H), 7.24 (d, J=2.4 Hz, 1H), 7.07 (dd, J=8.8, 2.4 Hz, 1H), 4.91-4.67 (m, 1H), 4.67-4.45 (m, 1H), 4.01-3.90 (m, 1H), 3.77-3.66 (m, 2H), 3.66-3.47 (m, 2H), 3.09 (d, J=4.4 Hz, 3H), 2.77-2.66 (m, 1H), 2.44-2.35 (m, 1H), 2.35-2.28 (m, 1H), 1.87-1.71 (m, 1H), 1.26-1.12 (m, 5H).

HNMR for Example 412: ¹H NMR (400 MHz, DMSO-d₆) δ 11.09 (s, 1H), 9.61 (s, 1H), 9.44 (s, 1H), 8.89 (s, 1H), 8.70-8.57 (m, 1H), 7.58 (d, J=8.8 Hz, 1H), 7.2.4 (d, J=2.4 Hz, 1H), 7.07 (dd, J=8.8, 2.4 Hz, 1H), 4.91-4.67 (m, 1H), 4.67-4.45 (m, 1H), 4.01-3.90 (m, 1H), 3.77-3.66 (m, 2H), 3.66-3.47 (m, 2H), 3.09 (d, J=4.4 Hz, 3H), 2.77-2.66 (m, 1H), 2.44-2.35 (m, 1H), 2.35-2.28 (m, 1H), 1.87-1.71 (m, 1H), 1.26-1.12 (m, 5H).

### Examples 413, 414, and 415

### Synthesis of 2-(difluoromethyl)-N-(8-methylamino)-5-(5-((S)-2-methylmorpholino benzo[d]oxazol-2-yl)-2,7-naphthyridin-3-yl)cyclopropane-1-carboxamide (cis racemate) (Example 413), (1S,2R)-2-(difluoromethyl)-N-(8-(methylamino)-5-(545)-2-methylmorpholino)benzo[d]oxazol-2-yl)-2,7-naphthyridin-3-yl)cyclopropane-1-carboxamide (Example 414) and (1R,2S)-2-(difluoromethyl)-N-(8-(methylamino)-5-(5-((S)-2-methylmorpholino)benzo[d]oxazol-2-yl)-2,7-naphthyridin-3-yl)cyclopropane-1-carboxamide (Example 415)

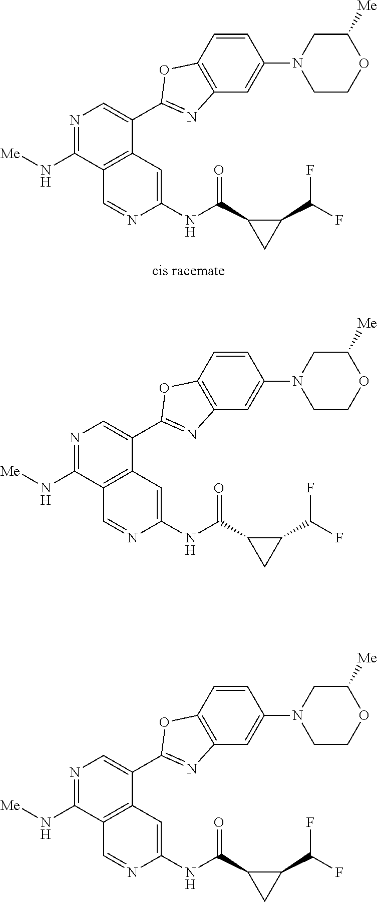 [SMILES: CNc1ncc(-c2nc3cc(N4CCO[C@@H](C)C4)ccc3o2)c2cc(NC(=O)[C@@H]3C[C@@H]3C(F)F)ncc12 ; CNc1ncc(-c2nc3cc(N4CCO[C@@H](C)C4)ccc3o2)c2cc(NC(=O)[C@H]3C[C@H]3C(F)F)ncc12 ; CNc1ncc(-c2nc3cc(N4CCO[C@@H](C)C4)ccc3o2)c2cc(NC(=O)[C@@H]3C[C@@H]3C(F)F)ncc12]

### Step 1: 2-(difluoromethyl)-N-(8-(methylamino)-5-(5-((8)-2-methylmorpholino)benzo[d]oxazol-2-yl)-2,7-naphthyridin-3-yl)cyclopropane-1-carboxamide (cis casemate)

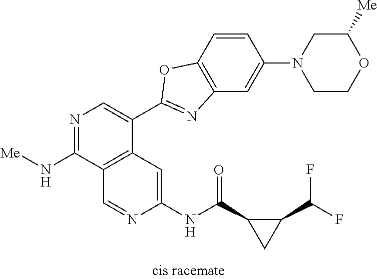 [SMILES: CNc1ncc(-c2nc3cc(N4CCO[C@@H](C)C4)ccc3o2)c2cc(NC(=O)[C@@H]3C[C@@H]3C(F)F)ncc12]

2(difluoromethyl)-N-(8-(methylamino)-5-(5-((S)-2-methylmorpholino)benzo[d]oxazol-2-yl)-2,7-naphthyridin-3-yl)cyclopropane-1-carboxamide (cis racemate) was synthesized using a similar procedure that was previously described in Example 407, by using (S)—N¹-methyl-4-(5-(2-methylmorpholino)benzo[d]oxazol-2-yl)-2,7-naphthyridine-1,6-diamine and (difluoromethyl)cyclopropane-1-carboxylic acid (cis racemate) as the starting material. LCMS (ESI) m/z 509.2 [M+H]⁺. ¹H NMR (400 MHz, DMSO-d₆) δ 11.27 (s, 1H), 9.61 (s, 1H), 9.46 (s, 1H), 8.90 (s, 1H), 8.69-8.62 (m, 1H), 7.59 (d, J=8.8 Hz, 1H), 7.25 (d, J=2.0 Hz, 1H), 7.11-7.03 (m, 1H), 6.27-5.92 (m, 1H), 3.99-3.91 (m, 1H), 3.74-3.60 (m, 3H), 3.55-3.50 (m, 1H), 3.09 (d, J=4.4 Hz, 3H), 2.76-2.69 (m, 1H), 2.48-2.37 (m, 2H), 1.99-1.88 (m, 1H), 1.41-1.30 (m, 2H), 1.19 (d, J=6.4 Hz, 3H).

### Step 2: (1S,2R)-2-(difluoromethyl)-N-(8-(methylamino)-5-(5-((S)-2-methylmorpholino)benzo[d]oxazol-2-yl)-2,7-naphthyridin-3-yl)cyclopropane-1-carboxamide and (1R,2S)-2-(difluoromethyl)-N-(8-(methylamino)-5-(5-((5)-2-methylmorpholino)benzo[d]oxazol-2-yl)-2,7-naphthyridin-3-yl)cyclopropane-1-carboxamide

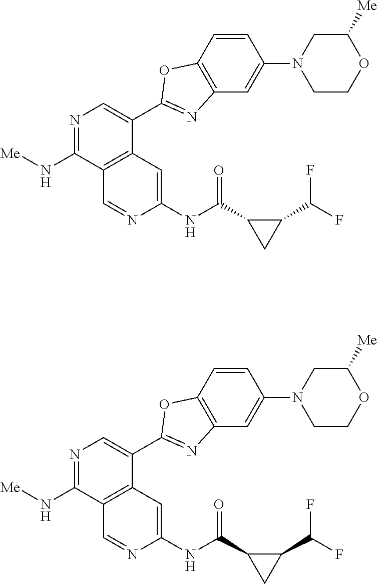 [SMILES: CNc1ncc(-c2nc3cc(N4CCO[C@@H](C)C4)ccc3o2)c2cc(NC(=O)[C@H]3C[C@H]3C(F)F)ncc12 ; CNc1ncc(-c2nc3cc(N4CCO[C@@H](C)C4)ccc3o2)c2cc(NC(=O)[C@@H]3C[C@@H]3C(F)F)ncc12]

2-(difluoromethyl)-N-(8-(methylamino)-5-(5-((S)-2-methylmorpholino)benzo[d]oxazol-2-yl)-2,7-naphthyridin-3-yl)cyclopropane-1-carboxamide (cis racemate) was separated by Chiral-HPLC (Column: Chiral ART Cellulose-SA, 2×25 cm, 5 μm; Mobile Phase A: Hex (0.5% 2 M NH₃-MeOH); Mobile Phase B: EtOH: CH₂Cl₂=1:1; Flow rate: 20 mL/min; Gradient: 30% B to 30% B in 25 min; Wave Length: 220/254 nm; R_{T1}(min): 19.75; R_{T2}(min): 23.65) to afford (1S,2R)-2-(difluoromethyl)-N-(8-(methylamino)-5-(5-((S)-2-methylmorpholino)benzo[d]oxazol-2-yl)-2,7-naphthyridin-3-yl)cyclopropane-1-carboxamide (Example 414, the faster peak) as a yellow solid (18.5 mg, 10.0%) and (1R,2S)-2-(difluoromethyl)-N-(8-(methylamino)-5-(5-((S)-2-methylmorpholino)benzo[d]oxazol-2-yl)-2,7-naphthyridin-3-yl)cyclopropane-1-carboxamide (Example 415, the slower peak) as a yellow solid (28.2 mg, 18.0%). The two configurations are arbitrarily assigned. LCMS (ESI) m/z 509.2 [M+H]⁺.

HNMR for Example 414: ¹H NMR (400 MHz, DMSO-d₆) δ 11.28 (s, 1H), 9.61 (s, 1H), 9.46 (s, 1H), 8.90 (s, 1H), 8.70-8.60 (m, 1H), 7.59 (d, J=8.8 Hz, 1H), 7.25 (d, J=2.4 Hz, 1H), 7.10-7.02 (m, 1H), 6.25-5.91 (m, 1H), 3.99-3.91 (m, 1H), 3.74-3.60 (m, 3H), 3.55-3.51 (m, 1H), 3.09 (d, J=4.4 Hz, 3H), 2.78-2.67 (m, 1H), 2.49-2.38 (m, 2H), 1.99-1.89 (m, 1H), 1.43-1.30 (m, 2H), 1.19 (d, J=6.0 Hz, 314).

HNMR for Example 415: ¹H NMR (400 MHz, DMSO-d₆) δ 11.27 (s, 1H), 9.61 (s, 1H), 9.46 (s, 1H), 8.90 (s, 1H), 8.70-8.62 (m, 1H), 7.59 (d, J=8.8 Hz, 1H), 7.25 (d, J=2.0 Hz, 1H), 7.10-7.01 (m, 1H), 6.24-5.90 (m, 1H), 3.98-3.91 (m, 1H), 3.75-3.60 (m, 3H), 3.55-3.51 (m, 1H), 3.09 (d, J=4.4 Hz, 3H), 2.70-2.70 (m, 1H), 2.48-2.36 (m, 2H), 1.99-1.88 (m, 1H), 1.42-1.30 (m, 2H), 1.19 (d, J=6.0 Hz, 3H).)

### Example 416: Synthesis of 2-(2-hydroxypropan-2-yl)-N-(8-(methylamino)-5-(5-((5)-2-methylmorpholino)benzo[d]oxazol-2-yl)-2,7-naphthyridin-3-yl)cyclopropane-1-carboxamide (cis racemate)

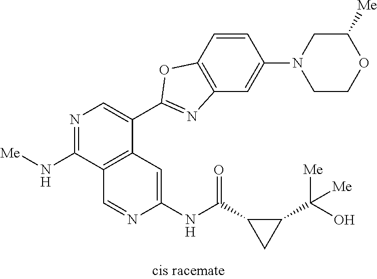 [SMILES: CNc1ncc(-c2nc3cc(N4CCO[C@@H](C)C4)ccc3o2)c2cc(NC(=O)[C@H]3C[C@H]3C(C)(C)O)ncc12]

### Step 1: ethyl 2-((8-(methylamino)-5-(5-((S)-2-methylmorpholino)benzo[d]oxazol-2-yl)-2,7-naphthyridin-3-yl)carbamoyl)cyclopropane-1-carboxylate (cis racemate)

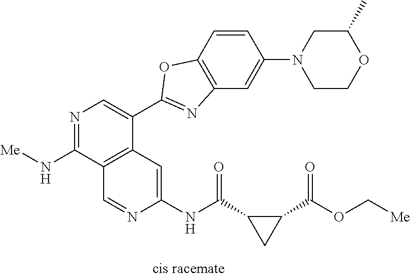 [SMILES: CCOC(=O)[C@@H]1C[C@@H]1C(=O)Nc1cc2c(-c3nc4cc(N5CCO[C@@H](C)C5)ccc4o3)cnc(NC)c2cn1]

To a stirred solution of (S)—N¹-methyl-4-(5-(2-methylmorpholino)benzo[d]oxazol-2-yl)-2,7-naphthyridine-1,6-diamine (100.0 mg; 0.256 mmol; 1.00 eq.) and 2-(ethoxycarbonyl)cyclopropane-1-carboxylic acid (cis racemate) (36.4 mg; 0.230 mmol; 0.90 eq.) in pyridine (4 mL) was added POCl₃ (117.8 mg; 0.768 mmol; 3.00 eq.) drop-wise at 0° C. under nitrogen atmosphere. The resulting mixture was stirred at room temperature for 0.5 hour. The reaction was quenched by the addition of water (10 mL), extracted with EtOAc (3×10 mL). The combined organic layers were washed with brine (2×10 mL), dried over anhydrous Na₂SO₄. After filtration, the filtrate was concentrated under reduced pressure. The residue was purified by flash chromatography on silica gel column using 0-10% of MeOH in CH₂Cl₂ as eluent to provide ethyl ethyl 2((8-(methylamino)-5-(5((S)-2-methylmorpholino)benzo[d]oxazol-2-yl)-2,7-naphthyridin-3-yl)carbamoyl)cyclopropane-1-carboxylate (cis racemate) as a yellow solid (70.0 mg, 51.5%). LCMS (ESI) m/z 531.2, [M+H]⁺.

### Step 2: 2-(2-hydroxypropan-2-yl)-N-(8-(methylamino)-5-(5-((S)-2-methylmorpholino)benzo[d]oxazol-2-yl)-2,7-naphthyridin-3-yl)cyclopropane-1-carboxamide (cis racemate)

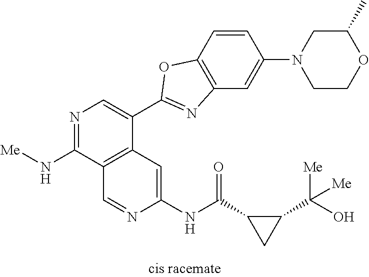 [SMILES: CNc1ncc(-c2nc3cc(N4CCO[C@@H](C)C4)ccc3o2)c2cc(NC(=O)[C@H]3C[C@H]3C(C)(C)O)ncc12]

To a stirred solution of ethyl 2-((8-(methylamino)-5-(5-((S)-2-methylmorpholino)benzo[d]oxazol-2-yl)-2,7-naphthyridin-3-yl)carbamoyl)cyclopropane-1-carboxylate (cis racemate) (60.0 mg; 0.113 mmol; 1.00 eq.) in THF (5 mL) was added methylmagnesium bromide (1 M in THF; 0.3 mL) dropwise at 0° C. under nitrogen atmosphere. The resulting mixture was stirred at room temperature for 3 hours. The reaction was quenched by the addition of water mL), extracted with EtOAc (3×10 mL). The combined organic layers were washed with brine (2×7 mL), dried over anhydrous Na₂SO₄. After filtration, the filtrate was concentrated under reduced pressure. The residue was purified by flash chromatography on silica gel column using 0-10% of MeOH in CH₂Cl₂ as eluent to provide 2-(2-hydroxypropan-2-yl)-N-(8-(methylamino)-5-(5-((5)-2-methylmorpholino)benzo[d]0 oxazol-2-yl)-2,7-naphthyridin-3-yl)cyclopropane-1-carboxamide (cis racemate) as a yellow solid (41.4 mg, 69.8%). LCMS (ESI) m/z 517.2, [M+H]⁺. ¹H NMR (400 MHz, DMSO-d₆) δ 11.29 (s, 1H), 9.62 (s, 1H), 9.45 (s, 1H), 8.91 (s, 1H), 8.71-8.62 (m, 1H), 7.56 (d, J=8.8 Hz, 1H), 7.19 (d, J=2.4 Hz, 1H), 7.12-7.04 (m, 1H), 5.23 (s, 1H), 3.99-3.91 (m, 1H), 3.76-3.65 (m, 2H), 3.60 (d, J=11.6 Hz, 1H), 3.53-3.46 (m, 1H), 3.09 (d, J=4.4 Hz, 3H), 2.77-2.67 (m, 1H), 2.42-2.34 (m, 1H), 2.10-1.99 (m, 1H), 1.59-1.44 (m, 2H), 1.24 (d, 9.6 Hz, 6H), 1.20-1.14 (m, 4H).

### Example 417: Synthesis of (S)—N¹-methyl-N⁶-(1-methyl-1H-pyrazol-3-yl)-4-(5-(2-methylmorpholino)benzo[d]oxazol-2-yl)-2,7-naphthyridine-1,6-diamine

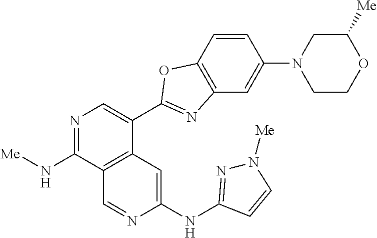 [SMILES: CNc1ncc(-c2nc3cc(N4CCO[C@@H](C)C4)ccc3o2)c2cc(Nc3ccn(C)n3)ncc12]

A solution of (S)—N¹-methyl-4-(5-(2-methylmorpholino)benzo[d]oxazol-2-yl)-2,7-naphthyridine-1,6-diamine (80.0 mg; 0.205 mmol; 1.00 eq.), 3-bromo-1-methylpyrazole (32.9 mg; 0.204 mmol; 1.00 eq.), EPhos Pd G₄ (18.8 mg; 0.020 mmol; 0.10 eq.), EPhos (10.9 mg; 0.020 mmol; 0.10 eq.) and Cs₂CO₃ (267.0 mg; 0.819 mmol; 4.00 eq.) in 1,4-dioxane (2 mL) was stirred at 120° C. for 3 hours under nitrogen atmosphere. The desired product was detected via LCMS. The resulting mixture was concentrated under reduced pressure. The residue was purified by flash chromatography on silica gel column using 5-10% of MeOH in CH₂Cl₂ as eluent to afford a crude product. The crude product was purified by preparative Achiral-SFC (Column: GreenSep Basic 3×15 cm, 5 urn; Mobile Phase A: CO₂, Mobile Phase B: IPA (0.1% 2 M NH₃-MeOH); Flow rate: 75 mL/min; Gradient: isocratic 38% B; Wave Length: 254 nm; R_{T1}(min): 6.03) to afford (S)—N¹-methyl-N⁶-(1-methyl-1H-pyrazol-3-yl)-4-(5-(2-methylmorpholino)benzo[d]oxazol-2-yl)-2,7-naphthyridine-1,6-diamine as a yellow solid (39.6 mg, 39.0%). LCMS (ESI) m/z 471.2, [M+H]⁺. ¹H NMR (400 MHz, DMSO-d₆) δ 9.61 (s, 1H), 9.27 (s, 1H), 9.04 (s, 1H), 8.78 (s, 1H), 8.48-8.35 (m, 1H), 7.63-7.55 (m, 2H), 7.23 (d, J=2.4 Hz, 1H), 7.10-7.02 (m, 1H), 6.19 (d, J=2.4 Hz, 1H), 3.99-3.91 (m, 1H), 3.86 (s, 3H), 3.79-3.65 (m, 2H), 3.64-3.58 (m, 1H), 3.55-3.46 (m, 1H), 3.06 (d, J=4.4 Hz, 3H), 2.77-2.68 (m, 1H), 2.42-2.33 (m, 1H), 1.19 (d, J=6.4 Hz, 3H).

### Example 418: Synthesis of (1S,2S)-2-fluoro-N-(8-(methylamino)-5-(5-((R)-2-methylmorpholino)benzo[d]oxazol-2-yl)-2,7-naphthyridin-3-yl)cyclopropane-1-carboxamide

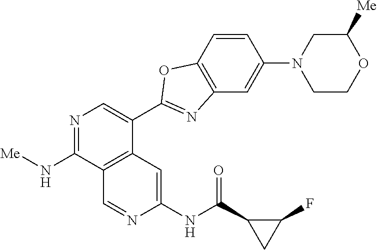 [SMILES: CNc1ncc(-c2nc3cc(N4CCO[C@H](C)C4)ccc3o2)c2cc(NC(=O)[C@@H]3C[C@@H]3F)ncc12]

### Step 1: (R)—N¹-methyl-4-(5-(2-methylmorpholino)benzo[d]oxazol-2-yl)-2,7-naphthyridine-1,6-diamine

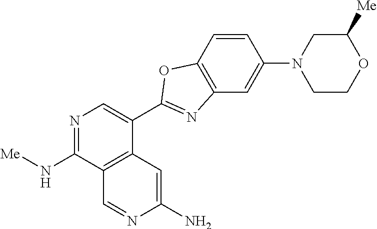 [SMILES: CNc1ncc(-c2nc3cc(N4CCO[C@H](C)C4)ccc3o2)c2cc(N)ncc12]

(R)—N¹-methyl-4-(5-(2-methylmorpholino)benzo[d]oxazol-2-yl)-2,7-naphthyridine-1,6-diamine was synthesized using a similar procedure that was previously described in Example 407, by using (R)—N-(8-(methylamino)-5-(5-(2-methylmorpholino)benzo[d]oxazol-2-yl)-2,7-naphthyridin-3-yl)cyclopropanecarboxamide (Example 302) as the starting material. LCMS (ESI) m/z 391.2, [M+H]⁺.

### Step (1S,2S)-2-fluoro-N-(8-(methylamino)-5-(5-((R)-2-methylmorpholino)benzo[d]oxazol-2-yl)-2,7-naphthyridin-3-yl)cyclopropane-1-carboxamide

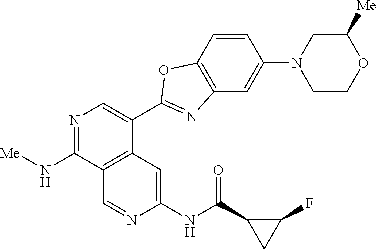 [SMILES: CNc1ncc(-c2nc3cc(N4CCO[C@H](C)C4)ccc3o2)c2cc(NC(=O)[C@@H]3C[C@@H]3F)ncc12]

To a solution of (R)—N¹-methyl-4-(5-(2-methylmorpholino)benzo[d]oxazol-2-yl)-2,7-naphthyridine-1,6-diamine (100.0 mg; 0.256 mmol; 1.00 eq.) and (1S,2S)-2-fluorocyclopropane-1-carboxylic acid (24.0 mg; 0.231 mmol; 0.90 eq.) in pyridine (5 mL) was added POCl₃ (114.5 mg; 0.747 mmol; 3.00 eq.) dropwise at 0° C. The resulting mixture was stirred at room temperature for 2 hours. The desired product was detected via LCMS. The solution was quenched by the addition of water (10 mL), extracted with EtOAc (3×50 mL). The combined organic layers were washed with brine (3×10 mL), dried over anhydrous Na₂SO₄. After filtration, the filtrate was concentrated under reduced pressure. The residue was purified by flash chromatography on silica gel column using 5-10% of MeOH in CH₂Cl₂ as eluent to afford a crude product. The residue was purified by flash chromatography on pre-packed C18 column using 50-80% of MeOH in water (10 mmol/L NH₄HCO₃) as eluent to provide (1S,2S)-2-fluoro-N-(8-(methylamino)-5-(5-((R)-2-methyl morpholino)benzo[d]oxazol-2-yl)-2,7-naphthyridin-3-yl)cyclopropane-1-carboxamide as a yellow solid (64.4 mg, 52.8%). LCMS (ESI) m/z 477.2, [M+H]⁺. ¹H NMR (400 MHz, DMSO-d₆) δ 11.07 (s, 1H), 9.65 (s, 1H), 9.45 (s, 1H), 8.89 (s, 1H), 8.77-8.48 (m, 1H), 7.59 (d, J=8.8 Hz, 1H), 7.24 (d, J=2.4 Hz, 1H), 7.15-7.89 (m, 1H), 5.13-4.78 (m, 1H), 3.98-3.89 (m, 1H), 3.78-3.60 (m, 3H), 3.55-3.46 (m, 1H), 3.09 (d, J=4.4 Hz, 3H), 2.77-2.66 (m, 1H), 2.45-2.37 (m, 1H), 2.37-2.26 (m, 1H), 1.82-1.63 (m, 1H), 1.31-1.10 (m, 4H).

### Example 419: Synthesis of (1S,2R)-2-methyl-N-(8-(methylamino)-5-(5-((R)-2-methylmorpholino)benzo[d]oxazol-2-yl)-2,7-naphthyridin-3-yl)cyclopropane-1-carboxamide

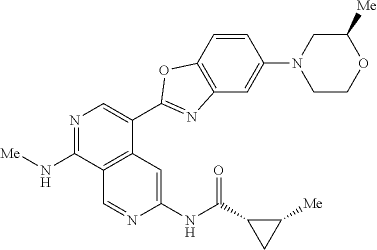 [SMILES: CNc1ncc(-c2nc3cc(N4CCO[C@H](C)C4)ccc3o2)c2cc(NC(=O)[C@H]3C[C@H]3C)ncc12]

To a stirred solution of (R)—N¹-methyl-4-(5-(2-methylmorpholino)benzo[d]oxazol-2-yl)-2,7-naphthyridine-1,6-diamine (250.0 mg; 0.640 mmol; 1.00 eq.) and (1S,2R)-2-methylcyclopropane-1-carboxylic acid (80 mg; 0.799 mmol; 1.25 eq.) in pyridine (5 mL) was added POCl₃ (350.0 mg; 2.283 mmol; 3.57 eq.) dropwise at 0° C. under nitrogen atmosphere. The resulting solution was stirred at room temperature for 1 hour. The reaction was quenched with water (5 mL), extracted with EtOAc (3>20 mL). The combined organic layer was dried over anhydrous sodium sulfate and concentrated under vacuum. The residue was purified by flash chromatography on silica gel column using 5-10% of MeOH in CH₂Cl₂ to afford the crude product. This crude product was purified by flash chromatography on pre-packed C18 column using 60-90% of MeCN in water (10 mmol/L NH₄HCO₃) to provide (1S,2R)-2-methyl-N-(8-(methylamino)-5-(5-((R)-2-methylmorpholino)benzo[d]oxazol-2-yl)-2,7-naphthyridin-3-yl)cyclopropane-1-carboxamide as a yellow solid (133.9 mg, 44.2%). LCMS (ESI) m/z 473.2, [M+H]⁺. ¹H NMR (400 MHz, DMSO-d₆) δ 10.91 (s, 1H), 9.60 (s, 1H), 9.43 (s, 1H), 8.88 (s, 1H), 8.67-8.59 (m, 1H), 7.58 (d, J=8.8 Hz, 1H), 7.24 (d, J=2.4 Hz, 1H), 7.07 (dd, J=8.8, 2.4 Hz, 1H), 3.98-3.90 (m, 1H), 3.75-3.59 (m, 3H), 3.56-3.47 (m, 1H), 3.09 (d, J=4.4 HZ, 3H), 2.77-2.67 (m, 1H), 2.43-2.37 (m, 1H), 2.15-2.07 (m, 1H), 1.36-1.28 (m, 1H), 1.22-1.13 (m, 6H), 1.06-0.99 (m, 1H), 0.94-0.85 (m, 1H).

### Example 420: Synthesis of (R)—N¹-methyl-4-(5-(2-methylmorpholino)benzo[d]oxazol-2-yl)-N⁶-(pyridin-2-yl)-2,7-naphthyridine-1,6-diamine

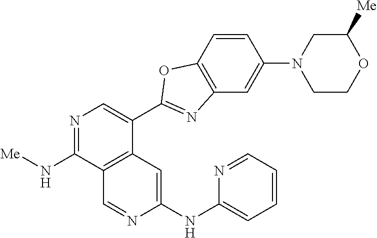 [SMILES: CNc1ncc(-c2nc3cc(N4CCO[C@H](C)C4)ccc3o2)c2cc(Nc3ccccn3)ncc12]

To a solution of 2-chloropyridine (23.2 mg; 0.204 mmol; 1.00 eq.) and (R)—N¹-methyl-4-(5-(2-methylmorpholino)benzo[d]oxazol-2-yl)-2,7-naphthyridine-1,6-diamine (80.0 mg; 0.205 mmol; 1.00 eq.) in dioxane (3 mL) were added Pd₂(dba)₃ (45.1 mg; 0.049 mmol; 0.24 eq.), XantPhos (57.0 mg; 0.099 mmol; 0.48 eq.) and Cs₂CO₃ (133.7 mg; 0.410 mmol; 2.00 eq.) at room temperature. The reaction was stirred at 130° C. for 3 hours under nitrogen atmosphere. The desired product was detected via LCMS. The resulting mixture was concentrated under vacuum. The residue was purified by flash chromatography on silica gel column using 2-10% of MeOH in CH₂Cl₂ as eluent to provide the crude product. The crude product was purified by flash chromatography on pre-packed C18 column using 20-70% of MeCN in water (10 mmol/L NH₄HCO₃) to provide (R)—N₁-methyl-4-(5-(2-methylmorpholino)benzo[d]oxazol-2-yl)-N⁶-(pyridin-2-yl)-2,7-naphthyridine-1,6-diamine as a yellow solid (28.0 mg, 29.2%). LCMS (ESI) m/z 468.2, [M+H]⁺. ¹H NMR (400 MHz, DMSO-d₆) δ 10.07 (s, 1H), 9.66 (s, 1H), 9.37 (s, 1H), 8.85 (s, 1H), 8.53-8.47 (m, 1H), 8.42-8.36 (m, 1H), 7.76-7.67 (m, 1H), 7.61 (d, J=8.8 Hz, 1H), 7.50-7.43 (m, 1H), 7.27 (d, J=2.4 Hz, 1H), 7.11-7.03 (m, 1H), 7.01-6.93 (m, 1H), 4.00-3.92 (m, 1H), 3.78-3.66 (m, 2H), 3.62-3.58 (m, 1H), 3.55-3.49 (m, 1H), 3.09 (d, J=4.4 Hz, 3H), 2.79-2.68 (m, 1H), 2.46-2.36 (m, 1H), 1.19 (d, J=6.2 Hz, 3H).

### Example 421: Synthesis of (S)-2,2-difluoro-N-(8-(methylamino)-5-morpholinobenzo[d]oxazol-2-yl)-2,7-naphthyridin-3-yl)cyclopropane-1-carboxamide

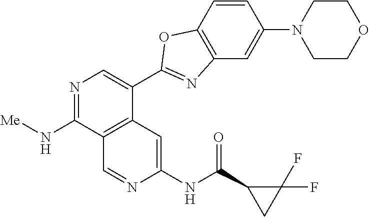 [SMILES: CNc1ncc(-c2nc3cc(N4CCOCC4)ccc3o2)c2cc(NC(=O)[C@@H]3CC3(F)F)ncc12]

### Step 1: N¹-methyl-4-5-morpholinobenzo[d]oxazol-2-yl)-2,7-naphthyridine-1,6-diamine

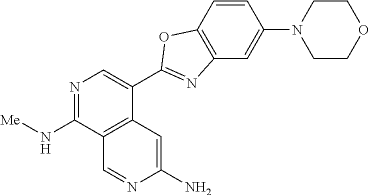 [SMILES: CNc1ncc(-c2nc3cc(N4CCOCC4)ccc3o2)c2cc(N)ncc12]

To a solution of N-(8-(methylamino)-5-(5-morpholinobenzo[d]oxazol-2-yl)-2,7-naphthyridin-3-yl)cyclopropanecarboxamide (Example 345) (1.20 g; 2.700 mmol; 1.00 eq.) in a mixture solvent of DMSO/THF (1:5, 60 mL) was added a solution of NaOH (1.08 g; 27.000 mmol; 10.00 eq.) in water (7 mL). The reaction was stirred at 60° C. overnight. The mixture was concentrated under vacuum to remove THF, to this mixture was added H₂O (250 mL), whereupon the precipitated product was isolated by filtration and washed with H₂O (3×20 mL) to provide N¹-methyl-4-(5-morpholinobenzo[d]oxazol-2-yl)-2,7-naphthyridine-1,6-diamine as a yellow solid (900.0 mg, 88.6%). LCMS (ESI) m/z 377.2, [M+H]⁺.

### Step 1: (S)-2,2-difluoro-N-(8-(methylamino)-5-(5-morpholinobenzo[d]oxazol-2-yl)-2,7-naphthyridin-3-yl)cyclopropane-1-carboxamide

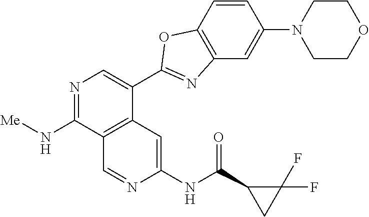 [SMILES: CNc1ncc(-c2nc3cc(N4CCOCC4)ccc3o2)c2cc(NC(=O)[C@@H]3CC3(F)F)ncc12]

A mixture of N¹-methyl-4-(5-morpholinobenzo[d]oxazol-2-yl)-2,7-naphthyridine-1,6-diamine (80.0 mg, 0.213 mmol, 1.00 eq) and (5)-2,2-difluorocyclopropane-1-carboxylic acid (23.4 mg, 0.192 mmol, 0.90 eq.) in pyridine 5.0 mL). To the above mixture was added POCl₃ (97.8 mg, 0.64 mmol, 3.00 equiv) dropwise at 0° C. The resulting mixture was stirred at room temperature for 1 hour. The reaction was quenched by the addition of H₂O (10 mL), extracted with EtOAc (3×50 mL). The combined organic layers were washed with brine (1×25 mL), dried over Na₂SO₄, filtered, and concentrated under reduced pressure. The residue was purified by flash chromatography on silica gel column using 2-10% of MeOH in CH₂Cl₂ as eluent to afford a crude product. The residue was purified by flash chromatography on pre-packed C18 column using 40-70% of MeOH in water (10 mmol/I, NH₄HCO₃) as eluent to provide (S)-2,2-difluoro-N-(8-(methylamino)-5-(5-morpholinobenzo[d]oxazol-2-yl)-2,7-naphthyridin-3-yl)cyclopropane-1-carboxamide (62.2 mg, 58.8%) as a yellow solid. LCMS (ESI) m/z 481.1, [M+H]⁺. ¹H NMR (400 MHz, DMSO-d₆) δ 11.25 (s, 1H), 9.67 (s, 1H), 9.46 (s, 1H), 8.91 (s, 1H), 8.71-8.60 (m, 1H), 7.59 (d, J=8.8 Hz, 1H), 7.23 (d, J=2.4 Hz, 1H), 7.11-7.02 (m, 1H), 3.82-3.75 (m, 4H), 3.20-3.13 (m, 4H), 3.13-3.01 (m, 4H), 2.19-2.01 (m, 2H).

### Examples 422-425

Each compound in Table 16 below was prepared using a similar experimental procedure to prepare Example 421, using ethyl-4-(5-morpholinobenzo[d]oxazol-2-yl)-2,7-naphthyridine-1,6-di amine as the common intermediate and appropriate acid.

TABLE 16

| Example | Structure and Name | MS and ¹H NMR |
|---|---|---|
| Example 422 | 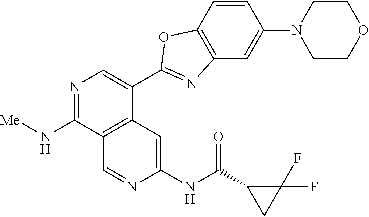 [SMILES: CNc1ncc(-c2nc3cc(N4CCOCC4)ccc3o2)c2cc(NC(=O)[C@H]3CC3(F)F)ncc12] | LCMS (ESI) m/z 481.2, [M + H]⁺. ¹H NMR (400 MHz, DMSO-d₆) δ 11.24 (s, 1H), 9.67 (s, 1H), 9.46 (s, 1H), 8.91 (s, 1H), 8.71-8.59 (m, 1H), 7.59 (d, J = 8.8 Hz, 1H), 7.23 (d, J = 2.4 Hz, 1H), 7.17-6.95 (m, 1H), 3.81- 3.77 (m, 4H), 3.20-3.14 (m, 4H), 3.10 (d, J = 4.4 Hz, 4H), 2.19-2.01 (m, 2H). |
| | (R)-2,2-difluoro-N-(8- | |
| | (methylamino)-5-(5- | |
| | morpholinobenzo[d]oxazol-2-y1)- | |
| | 2,7-naphthyridin-3- | |
| | yl)cyclopropane-1-carboxamide | |
| Example 423 | 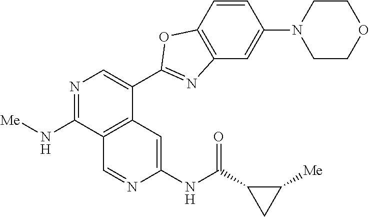 [SMILES: CNc1ncc(-c2nc3cc(N4CCOCC4)ccc3o2)c2cc(NC(=O)[C@H]3C[C@H]3C)ncc12] | LCMS (ESI) m/z 459.2, [M + H]⁺. ¹H NMR (400 MHz, DMSO-d₆) δ 10.92 (s, 1H), 9.62 (s, 1H), 9.43 (s, 1H), 8.88 (s, 1H), 8.66-8.58 (m, 1H), 7.59 (d, J = 8.8 Hz, 1H), 7.24 (d, J = 2.0 Hz, 1H), 7.11-7.02 (m, 1H), 3.82- 3.73 (m, 4H), 3.21-3.12 (m, 4H), 3.09 (d, J = 4.4 Hz, 3H), 2.16-2.09 (m, 1H), 1.36-1.27 (m, 1H), 1.17 (d, J = 6.4 Hz, 3H), 1.05-0.96 (m, 1H), 0.92-0.85 (m, 1H). |
| | (1S,2R)-2-methyl-N-(8- | |
| | (methylamino)-5-(5- | |
| | morpholinobenzo[d]oxazol-2-yl)- | |
| | 2,7-naphthyridin-3- | |
| | yl)cyclopropane-1-carboxamide | |
| Example 424 | 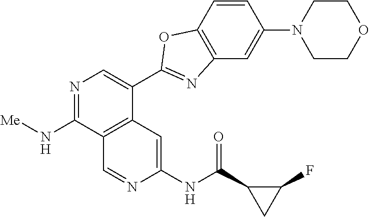 [SMILES: CNc1ncc(-c2nc3cc(N4CCOCC4)ccc3o2)c2cc(NC(=O)[C@@H]3C[C@@H]3F)ncc12] | LCMS (ESI) m/z 463.1, [M + H]⁺. ¹H NMR (400 MHz, DMSO-d₆) δ 11.08 (s, 1H), 9.66 (s, 1H), 9.44 (s, 1H), 8.90 (s, 1H), 8.69-8.61 (m, 1H), 7.59 (d, J = 8.8 Hz , 1H), 7.24 (d, J = 2.4 Hz ,1H), 7.10-7.03 (m, 1H), 5.10- 4.86 (m, 1H), 3.82-3.76 (m, 4H), 3.20-3.12 (m, 4H), 3.09 (d, J = 4.4 Hz, 3H), 2.36-2.28 (m, 1H), 1.80- 1.69 (m, 1H), 1.28-1.19 (m, 1H). |
| | (1S,2S)-2-fluoro-N-(8- | |
| | (methylamino)-5-(5- | |
| | morpholinobenzo[d]oxazol-2-yl)- | |
| | 2,7-naphthyridin-3- | |
| | yl)cyclopropane-1-carboxamide | |
| Example 425 | 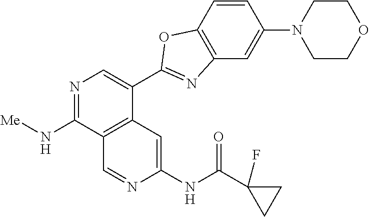 [SMILES: CNc1ncc(-c2nc3cc(N4CCOCC4)ccc3o2)c2cc(NC(=O)C3(F)CC3)ncc12] | LCMS (ESI) m/2 463.2, [M + H]⁺. ¹H NMR (400 MHz, DMSO-d₆) δ 10.42 (s, 1H), 9.68 (s, 1H), 9.49 (s, 1H), 8.93 (s, 1H), 8.79-8.70 (m, 1H), 7.59 (d, J = 9.2 Hz, 1H), 7.23 (d, J = 2.4 Hz, 1H), 7.11-6.99 (m, 1H), 3.86- 3.74 (m, 4H), 3.22-3.11 (m, 4H), 3.10 (d, J = 4.4 Hz, 3H), 1.54-1.41 (m, 4H). |
| | 1-fluoro-N-(8-(methylamino)-5-(5- | |
| | morpholinobenzo[d]oxazol-2-yl)- | |
| | 2,7-naphthyridin-3- | |
| | yl)cyclopropane-1-carboxamide | |

### Example 426: Synthesis of N⁶-(1,5-dimethyl-1/1-pyrazol-3-yl)-N¹-methyl-4-(5-morpholinobenzo[d]oxazol-2-yl)-2,7-naphthyridine-1,6-diamine

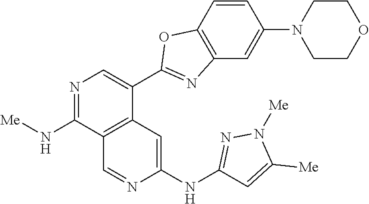 [SMILES: CNc1ncc(-c2nc3cc(N4CCOCC4)ccc3o2)c2cc(Nc3cc(C)n(C)n3)ncc12]

A solution of N¹-methyl-4-(5-morpholinobenzo[d]oxazol-2-yl)-2,7-naphthyridine-1,6-diamine (60.0 mg; 0.159 mmol; 1.00 eq.), 3-bromo-1,5-dimethyl-1H-pyrazole (27.9 mg, 0.159 mmol; 1.00 eq.), EPhos Pd G₄ (14.6 mg; 0.016 mmol; 0.10 eq.), EPhos (8.5 mg; 0.016 mmol; 0.10 eq.) and Cs₂CO₃ (207.7 mg: 0.636 mmol; 4.00 eq.) in 1,4-dioxane (4 mL) was stirred at 100° C. for 5 hours under nitrogen atmosphere. The desired product was detected via LCMS. The resulting mixture was concentrated under vacuum. The residue was purified by flash chromatography on silica gel column using 0-10% of Maki: in CH₂Cl₂ as eluent to afford N₆-(1,5-dimethyl-1H-pyrazol-3-yl)-N¹-methyl-4-(5-morpholinobenzo[d]oxazol-2-yl)-2,7-naphthyridine-1,6-diamine as a yellow solid (35.0 mg, 45.1%). LCMS (ESI) m/z 471.2, [M+H]⁺. ¹H NMR (400 MHz, DMSO-d₆) δ 9.48 (s, 1H), 9.25 (s, 1H), 9.05 (s, 1H), 8.77 (s, 1H), 8.40-8.35 (m, 1H), 7.59 (d, J=8.8 Hz, 1H), 7.22 (d, J=2.4 Hz, 1H), 7.06 (dd, J=8.8, 2.4 Hz, 1H), 6.02 (s, 1H), 3.82-3.76 (m, 4H), 3.74 (s, 3H), 3.20-3.11 (m, 4H), 3.06 (d, J=4.4 Hz, 3H), 2.28 (s, 3H).

### Example 427: Synthesis of 4-(benzo[d]oxazol-2-yl)-1H-methyl-N⁶-(pyridin-2-yl)-2,7-naphthyridine-1,6-diamine

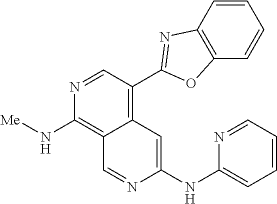 [SMILES: CNc1ncc(-c2nc3ccccc3o2)c2cc(Nc3ccccn3)ncc12]

### Step 1: 2-tributylstannyl)benzo[d]oxazole

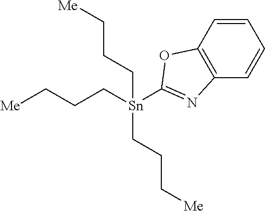 [SMILES: CCC[CH2][Sn]([CH2]CCC)([CH2]CCC)[c]1nc2ccccc2o1]

To a solution of benzo[d]oxazole (5.00 g; 41.97 mmol; 1.00 eq.) in THF (250 mL) was added n-butyllithium solution (2.5 M in hexane, 25 mL) dropwise at −78° C. under nitrogen atmosphere. The reaction mixture was stirred at −78° C. for 1 hour, to this solution was added tributylchlorostannane (14.00 g; 43.01 mmol; 1.02 eq.) dropwise and the mixture was stirred −78° C. for another 0.5 hour. The resulting mixture was diluted with hexane (150 mL), washed with brine (3×10 mL) and KF (10% w/w) (3×10 mL). The organic layer was dried over anhydrous Na₂SO₄ and concentrated under reduced pressure to afford 2-(tributylstannyl)benzo[d]oxazole as a black oil (17.3 g, crude). The crude product was used in the next step without further purification. LCMS (ESI) m/z 410.1, [M+H]⁺.

### Step 2: 4-(benzo[d]oxazol-2-yl)-6-chloro-N-methyl-2,7-naphthyridin-1-amine

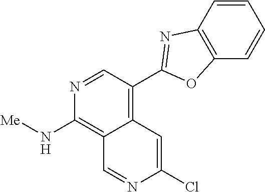 [SMILES: CNc1ncc(-c2nc3ccccc3o2)c2cc(Cl)ncc12]

To a stirred solution of 2-(tributylstannyl)benzo[d]oxazole (1.28 g; 3.136 mmol; 5.01 eq.) and 6-chloro-4-iodo-N-methyl-2,7-naphthyridin-1-amine (200.0 mg; 0.626 mmol; 1.00 eq.) in dioxane (5 mL) was added Pd(PPh₃)₂Cl₂(88.0 mg; 0.125 mmol; 0.20 eq.) and CuI (17.8 mg; 0.093 mmol; 0.15 eq.). The resulting mixture was stirred at 110° C. for 2 hours under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide 4-(benzo[d]oxazol-2-yl)-6-chloro-N-methyl-2,7-naphthyridin-1-amine as a yellow solid (150.0 mg, 77.1%). LCMS (ESI) m/z 311.1, [M+H]⁺.

### Step 3: 4-(benzo[d]oxazol-2-N¹-(pyridin-2-yl)-2,7-naphthyridine-1,6-diamine

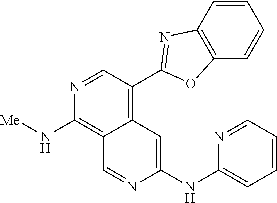 [SMILES: CNc1ncc(-c2nc3ccccc3o2)c2cc(Nc3ccccn3)ncc12]

To a stirred solution of 4-(benzo[d]oxazol-2-yl)-6-chloro-N-methyl-2,7-naphthyridin-1-amine (150.0 mg; 0.483 mmol, 1.00 eq.) and pyridin-2-amine (135.9 mg; 1.445 mmol; 2.99 eq.) in dioxane (17 mL) was added Pd₂(dba)₃ (50.3 mg; 0.055 mmol; 0.11 eq.), XantPhos (56.1 mg; 0.097 mmol; 0.20 eq.) and Cs₂CO₃ (315.0 mg; 0.967 mmol; 2.00 eq.). The resulting mixture was stirred at 110° C. for 4 hours under nitrogen atmosphere. Upon completion, the resulting mixture was concentrated under reduced pressure. The residue was purified by flash chromatography on silica gel column using 0-10% of MeOH in CH₂Cl₂ as eluent to provide a crude product. The residue was purified by flash chromatography on pre-packed C18 column using 40-70% of MeOH in water (10 mmol/L NH₄HCO₃) as eluent to provide 4-(benzo[d]oxazol-2-yl)-N¹-methyl-N⁶-(pyridin-2-yl)-2,7-naphthyridine-1,6-diamine as a yellow solid (143.8 mg, 80.8%). LCMS (ESI) m/z 369.1, [M+H]⁺. ¹H NMR (400 MHz, DMSO-d₆) δ 10.09 (s, 1H), 9.77 (s, 1H), 9.36 (s, 1H), 8.89 (s, 1H), 8.58-8.47 (m, 1H), 8.45-8.36 (m, 1H), 7.85-7.68 (m, 3H), 7.47-7.35 (m, 3H), 7.01-6.93 (m, 1H), 3.08 (d, J=4.4 Hz, 3H).

### Example 428: Synthesis of N-(5-(benzo[d]oxazol-2-yl)-8-(methylamino)-2,7-naphthyridin-3-yl)propionamide

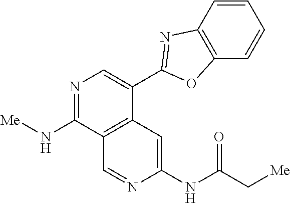 [SMILES: CCC(=O)Nc1cc2c(-c3nc4ccccc4o3)cnc(NC)c2cn1]

To a stirring mixture of 4-(benzo[d]oxazol-2-yl)-6-chloro-N-methyl-2,7-naphthyridin-1-amine (Example 427, step 2) (60.0 mg; 0.193 mmol; 1 eq.) in 1,4-dioxane (6 mL) were added propionamide (28.2 mg; 0.386 mmol; 2.00 eq.), Pd₂(dba)₃ (17.6 mg; 0.019 mmol; 0.10 eq.), XantPhos (22.3 mg; 0.039 mmol; 0.20 eq.) and Cs₂CO₃ (125.8 mg; 0.386 mmol; 2.00 eq.). The resulting mixture was stirred at 110° C. for 1 hour under nitrogen atmosphere. The desired product was detected via LCMS. The mixture was allowed to cool down to room temperature. The mixture was concentrated under reduced pressure. The residue was purified by flash chromatography on silica gel column using 5-10% of MeOH in CH₂Cl₂ as eluent to provide a crude product. The residue was purified by flash chromatography on pre-packed C18 column using 30-60% of MeOH in water (10 mmol/L NH₃HCO₃) as eluent to provide N-(5-(benzo[d]oxazol-2-yl)-8-(methylamino)-2,7-naphthyridin-3-yl)propionamide as a yellow solid (37.5 mg, 55.9%). LCMS (ESI) m/z 348.1, [M+H]⁺. ¹H NMR (400 MHz, DMSO-d₆) 10.70 (s, 1H), 9.73 (s, 1H), 9.43 (s, 1H), 8.93 (s, 1H), 8.69-8.63 (m, 1H), 7.80-7.74 (m, 2H), 7.41-7.39 (m, 2H), 3.09 (d, J=4.4 Hz, 3H), 2.51-2.48 (m, 2H), 1.15-1.11 (m, 3H).

### Example 429: Synthesis of N-(5-(benzo[d]oxazol-2-yl)-8-(methylamino)-2,7-naphthyridin-3-yl)isobutyramide

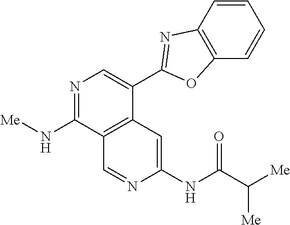 [SMILES: CNc1ncc(-c2nc3ccccc3o2)c2cc(NC(=O)C(C)C)ncc12]

A solution of 4-(benzo[d]oxazol-2-yl)-6-chloro-N-methyl-2,7-naphthyridin-1-amine (Example 427, step 2) (60.0 mg; 0.193 mmol; 1.00 eq.), isobutyramide (33.6 mg; 0.386 mmol; 2.00 eq.), Pd₂(dba)₂, (17.6 mg; 0.019 mmol; 0.10 eq.), XantPhos (22.3 mg; 0.039 mmol; 0.20 eq.) and Cs₂CO₃ (125.8 mg; 0.386 mmol; 2.00 eq.) in 1,4-dioxane (2 was stirred at 110° C. for 2 hours under nitrogen atmosphere. The desired product was detected via LCMS. The reaction was concentrated under pressure. The residue was purified by flash chromatography on silica gel column using MeOH in CH₂Cl₂ (0-5%) as eluent to provide a crude product. The crude product was dissolved in DMSO (1 mL), and was added dropwise into water (20 mL) and a precipitate formed. The solids were collected by filtration and washed with water (10 mL) to afford 1-(5-(benzo[d]oxazol-2-yl)-8-(methylamino)-2,7-naphthyridin-3-yl)isobutyramide as a light yellow solid (50.6 mg, 70.1%). LCMS (ESI) m/z 362.2, [M+H]⁺.

### Example 430: Synthesis of 4-(5-(benzyloxy)benzo[d]oxazol-2-yl)-N⁶-(5-(benzyloxy)pyridin-2-yl)-N¹-methyl-2,7-naphthyridine-1,6-diamine

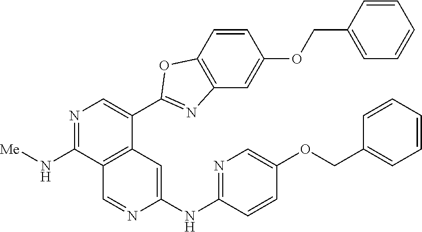 [SMILES: CNc1ncc(-c2nc3cc(OCc4ccccc4)ccc3o2)c2cc(Nc3ccc(OCc4ccccc4)cn3)ncc12]

### Step 1: 4-(5-(benzyloxy)benzo[d]oxazol-2-yl)-N¹-methyl-2,7-naphthyridine-1,6-diamine

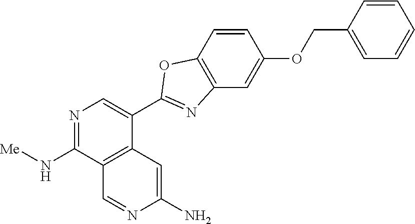 [SMILES: CNc1ncc(-c2nc3cc(OCc4ccccc4)ccc3o2)c2cc(N)ncc12]

To a solution of N-(5-(5-(benzyloxy)benzo[d]oxazol-2-yl)-8-(methylamino)-2,7-naphthyridin-3-yl)cyclopropanecarboxamide (Example 234) (80.0 mg 0.170 mmol; 1.00 eq.) in a mixture solvent of DMSO/MeOH (1:5, 4.8 mL) was added a solution of NaOH (68.8 mg; 1.720 mmol; 10.01 eq.) in water (1.3 ML) at 0° C. The reaction mixture was stirred at 60° C. overnight under nitrogen atmosphere. The desired product was detected via LCMS. The resulting mixture was concentrated under reduced pressure. The residue was purified by flash chromatography on pre-packed C18 column using 20-80% of MeCN in water (10 mmol/L NH₄HCO₃) as eluent to provide 4-(5-(benzyloxy)benzo[d]oxazol-2-yl)-M-methyl-2,7-naphthyridine-1,6-diamine as a yellow solid (60.0 mg, 87.8%). LCMS (ESI) m/z 398.2, [M+H]⁺.

### Step 2: 4-(5-(benzyloxy)benzo[d]oxazol-2-yl)-N⁶-(5-(benzyloxy)pyridin-2-yl)-N¹-methyl-2,7-naphthyridine-1,6-diamine

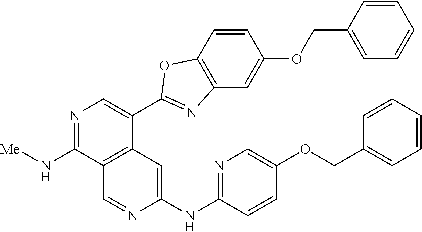 [SMILES: CNc1ncc(-c2nc3cc(OCc4ccccc4)ccc3o2)c2cc(Nc3ccc(OCc4ccccc4)cn3)ncc12]

To a stirring mixture of 4-(5-(benzyloxy)benzo[d]oxazol-2-yl)-N¹-methyl-2,7-naphthyridine-1,6-diamine (60.0 mg; 0.150 mmol; 1.00 eq.), 5-(benzyloxy)-2-chloropyridine (33.1 mg; 0.151 mmol; 1.00 eq.) in 1,4-dioxane (5 mL) were added Pd₂(dba)₃ (13.8 mg; 0.015 mmol; 0.10 eq.), XantPhos (17.5 mg; 0.030 mmol; 0.20 eq.) and Cs₂CO₃ (98.4 mg; 0.302 mmol 2.00 eq.). The resulting mixture was stirred at 130° C. for 2 hours under nitrogen atmosphere. The desired product was detected via LCMS. The resulting mixture was concentrated under reduced pressure. The residue was purified by flash chromatography on silica gel column using 0-20% of MeOH in CH₂Cl₂ as eluent to provide the crude product. The crude product was purified by flash chromatography on pre-packed C18 column using 20-80% of MeCN in water (10 mmol/L NH₄HCO₃) as eluent to provide 4-(5-(benzyloxy)benzo[d]oxazol-2-yl)-N⁶-(5-(benzyloxy)pyridin-2-yl)-N¹-methyl-2,7-naphthyridine-1,6-diamine as a yellow solid (24.0 mg, 27.1%). LCMS (ESI) m/z 581.2, [M+H]⁺.

### Example 431: Synthesis of N-(5-(benzo[d]oxazol-2-yl)-8-((methyl-d₃)amino)-2,7-naphthyridin-3-yl)acetamide

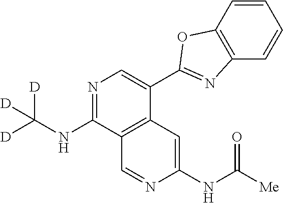 [SMILES: [2H]C([2H])([2H])Nc1ncc(-c2nc3ccccc3o2)c2cc(NC(C)=O)ncc12]

### Step 1: 6-chloro-iodo-N-(methyl-d₃)-2,7-naphthyridin-1-amine

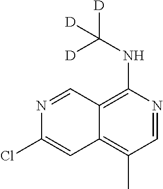 [SMILES: [2H]C([2H])([2H])Nc1ncc(C)c2cc(Cl)ncc12]

To a solution of 6-chloro-N-(methyl-d₃)-2,7-naphthyridin-1-amine (328.0 mg; 1.667 mmol; 1.00 eq.) in DMF (5 mL) was added NIS (562.7 mg, 2.501 mmol; 1.50 eq.) in portions at 0° C. under nitrogen atmosphere. The mixture was stirred at room temperature for 1 hour. The desired product was detected via LCMS. The resulting mixture was diluted with EtOAc (80 mL) and washed with brine (3×10 mL). The organic phase was dried with Na₂SO₄, concentrated under vacuum. The residue was slurried in DCM (5 mL) at room temperature for 3 hours to precipitate solids. The solids were collected by filtration to afford 6-chloro-4-iodo-N-(methyl-d₃)-2,7-naphthyridin-1-amine as a yellow solid (420.0 mg, 78.0%). LCMS (ESI) m/z 323.0, [M+H]⁺.

### Step 2: 4-(benzo[d]oxazol-2-yl)-6-chloro-N-(methyl-d₃)-2,7-naphthyridin-1-amine

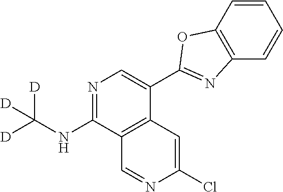 [SMILES: [2H]C([2H])([2H])Nc1ncc(-c2nc3ccccc3o2)c2cc(Cl)ncc12]

To a stirring mixture of 6-chloro-4-iodo-N-(methyl-d₃)-2,7-naphthyridin-1-amine (204.0 mg; 0.632 mmol; 1.00 eq.) and 2-(tributylstannyl)benzo[d]oxazole (1.30 g; 3.185 mmol; 5.04 eq.) in 1,4-dioxane (17 mL) was added CuI (18.0 mg; 0.094 mmol; 0.15 eq.) and Pd(PPh₃)₂Cl₂ (88.7 mg; 0.126 mmol; 0.2 eq.) under nitrogen atmosphere. After stirring at 110° C. for 2 hours, the desired product was detected via LCMS. The resulting mixture was concentrated under reduced pressure. The residue was purified by flash chromatography on silica gel column using 100% of EtOAc as eluent to provide 4-(benzo[d]oxazol-2-yl)-6-chloro-N-(methyl-d₃)-2,7-naplathyridin-1-amine as a yellow solid (160.0 mg, 80.6%). LCMS (ESI) m/z 314.1, [M+H]⁺.

### Step 3: N-(5-(benzo[d]oxazol-2-yl)-8-((methyl-d₃)amino)-2,7-naphthyridin-3-yl)acetamide

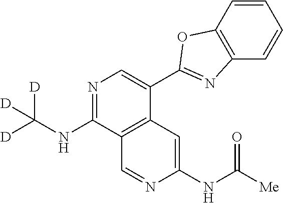 [SMILES: [2H]C([2H])([2H])Nc1ncc(-c2nc3ccccc3o2)c2cc(NC(C)=O)ncc12]

To a stirring mixture of 4-(benzo[d]oxazol-2-yl)-6-chloro-N-(methyl-d₃)-2,7-naplathyridin-1-amine (70.0 mg; 0.223 mmol; 1.00 eq.), acetamide (20.0 mg, 0.338 mmol; 1.52 eq.), XantPhos (25.8 mg; 0.044 mmol; 0.20 eq.) in 1,4-dioxane (7 mL) were added Cs₂CO₃ (146.0 mg; 0.448 mmol; 2.01 eq.) and Pd₂(dba)₃ (22.4 mg; 0.024 mmol; 0.11 eq.) under nitrogen atmosphere. After stirring at 110° C. for 4 hours, the desired product was detected via LCMS. The resulting mixture was concentrated under reduced pressure. The residue was purified by flash chromatography on silica gel column using 0-10% of MeOH in CH₂Cl₂ as eluent to provide a crude product. The crude product was purified by flash chromatography on pre-packed. C18 column using 0-100% of MeOH in water (10 mmol/L NH₄HCO₃) to provide N-(5-(benzo[d]oxazol-2-yl)-8-((methyl-d₃)amino)-2,7-naphthyridin-3-yl)acetamide as a yellow solid (29.0 mg, 38.6%). LCMS (ESI) m/z 337.1, [M+H]⁺. ¹H NMR (400 MHz, DMSO-d₆) δ 10.75 (s, 1H), 9.73 (s, 1H), 9.43 (s, 1H), 8.93 (s, 1H), 8.64 (s, 1H), 7.81-7.70 (m, 2H), 7.45-7.35 (m, 2H), 2.18 (s, 3H).

### Example 432: Synthesis of N-(5-(benzo[d]oxazol-2-yl)-8-((methyl-d₃)amino)-2,7-naphthyridin-3-yl)acetamide-2,2,2-d₃

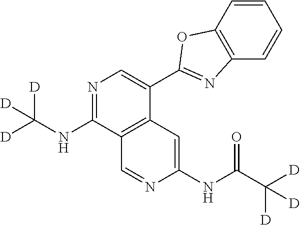 [SMILES: [2H]C([2H])([2H])Nc1ncc(-c2nc3ccccc3o2)c2cc(NC(=O)C([2H])([2H])[2H])ncc12]

### Step 1: N-(2,4-dimethoxybenzyl)acetamide-2,2,2-d₃

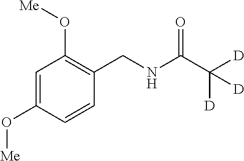 [SMILES: [2H]C([2H])([2H])C(=O)NCc1ccc(OC)cc1OC]

A solution of acetic-2,2,2-d₃ acid (2.00 g; 31.213 mmol; 1.00 eq.) and HATU (14.20 g; 37.345 mmol; 1.2.0 eq.) in DMF (50 mL) was stirred at room temperature for 20 minutes under nitrogen atmosphere. To the above solution were added DIPEA (12.00 g; 92,845 mmol; 2.97 eq.) and (2,4-dimethoxyphenyl)methanamine (10.40 g; 62,198 mmol; 1.99 eq.). The resulting solution was stirred at room temperature for 2 hours under nitrogen atmosphere. The desired product was detected via LCMS. The resulting mixture was diluted with EtOAc (300 mL) and washed with brine (5×50 mL). The organic phase was dried with Na₂SO₄ and concentrated under reduced pressure. The residue was purified by flash chromatography on pre-packed C18 column using 10-30% of MeCN in water (10=ion NH₄HCO₃) as eluent to provide N-(2,4-dimethoxybenzyl)acetamide-2,2,2-d₃ as a light yellow solid (4.00 g, 60.3%). LCMS (ESI) m/z 213.1, [M+H]⁺.

### Step 2: acetamide-2,2,2-d₃

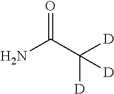

A solution of N-(2,4-dimethoxybenzyl)acetamide-2,2,2-d₃(1.00 g; 4.711 mmol; 1.00 eq.) in TFA (10 mL) was stirred at 70° C. for 1 hour. The desired product was detected via LCMS. The resulting mixture was concentrated under reduced pressure. The residue was purified by flash chromatography on silica gel column using 0-15% of MeOH in CH₂Cl₂ as eluent to provide acetamide-2,2,2-d₃ as a light-yellow solid (290.0 mg, 99.3%). LCMS (PSI) m/z 63.1 [M+H]⁺.

### Step 3: N-(5-(benzo[d]oxazol-2-yl)-8-((methyl-d)amino)-2,7-naphthyridin-3-yl)acetamide-2,2,2-d₃

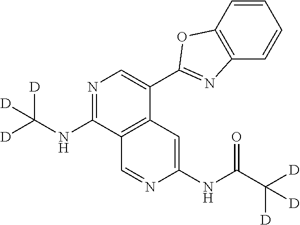 [SMILES: [2H]C([2H])([2H])Nc1ncc(-c2nc3ccccc3o2)c2cc(NC(=O)C([2H])([2H])[2H])ncc12]

A solution of 4-(benzo[d]oxazol-2-yl)-6-chloro-N-(methyl-d₃)-2,7-naphthyridin-1-amine (Example 431, step 2) (60.0 mg; 0.191 mmol; 1.00 eq.), Pd₂(dba)₃ (17.5 mg; 0.019 mmol; 0.10 eq.), Xantphos (22.1 mg; 0.038 mmol; 0.20 eq.), Cs₂CO₃ (124 mg; 0.381 mmol; 1.99 eq.) and acetamide-2,2,2-d₃(47.4 mg; 0.763 mmol; 3.99 eq.) in 1,4-dioxane (5 mL) was stirred at 110° C. for 4 hours under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide the crude product. The crude product was repurified by C18 column using 30-60% of MeCN in water (10 mmol/L, NH₄HCO₃) as eluent to provide N-(5-(benzo[d]oxazol-2-yl)-8-((methyl-d₃)amino)-2,7-naphthyridin-3-yl)acetamide-2,2,2-d₃ as a light yellow solid (15.8 mg, 24.3%). LCMS (ESI) m/z 340.2, [M+H]⁺. ¹H NMR (400 MHz, DMSO-d₆) δ 10.73 (s, 1H), 9.72 (s, 1H), 9.43 (s, 1H), 8.93 (s, 1H), 8.65-8.59 (m, 1H), 7.79-7.70 (m, 2H), 7.44-7.35 (m, 2H)

### Examples 433 and 434

Synthesis of N-(5-(benzo[d]oxazol-2-yl)-8-((methyl-d₃)amino)-2,7-naphthyridin-3-yl)-2-fluorocyclopropane-1-carboxamide (cis racemate) (Example 433) and N-(5-(benzo[d]oxazol-2-yl)-8-((methyl-d₃)amino)-2,7-naphthyridin-3-yl)-2-fluorocyclopropane-1-carboxamide (trans racemate) (Example 434)

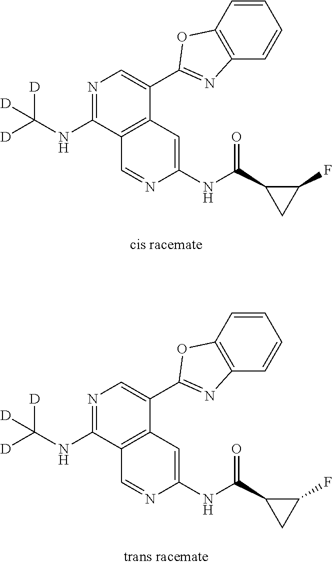 [SMILES: [2H]C([2H])([2H])Nc1ncc(-c2nc3ccccc3o2)c2cc(NC(=O)[C@@H]3C[C@@H]3F)ncc12 ; [2H]C([2H])([2H])Nc1ncc(-c2nc3ccccc3o2)c2cc(NC(=O)[C@@H]3C[C@H]3F)ncc12]

### Step 1: 2-fluorocyclopropane-1-carboxamide (cis racemate

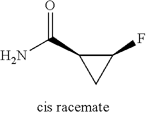

To a solution of 2-fluorocyclopropane-1-carboxylic acid (cis racemate) (1.00 g; 9.608 mmol, 1.00 eq.) in CH₂Cl₂ (30 mL) was added DMF (152.0 mg; 2.079 mmol; 0.22 eq.) at room temperature and oxalyl chloride (2 M in CH₂Cl₂, 50.8 mL) at 0° C. The reaction was stirred at room temperature for 1 hour under nitrogen atmosphere. The resulting mixture was concentrated under vacuum. The residue was dissolved in CH₂Cl₂ (3 mL) and added dropwise into a stirred solution of NH₃ (gas) (7 M in MeOH, 20 mL) at 0° C. under nitrogen atmosphere. The reaction was stirred at room temperature for 1 hour. Upon completion, the resulting mixture was concentrated under vacuum. The residue was purified by flash chromatography on silica gel column using 2-10% of MeOH in CH₂Cl₂ as eluent to provide 2-fluorocyclopropane-1-carboxamide (cis racemate) as a white solid (416.0 ring, 42.0%), LCMS (ESI) m/z 104.0, [M+H]⁺.

### Step 2: N-(5-(benzo[d]oxazol-2-yl)-8-((methyl-d₃)amino)-2,7-naphthyridin-3-yl)-2-fluorocyclopropane-1-carboxamide (cis racemate) and N-(5-(benzo[d]oxazol-2-yl)-8-((methyl-d₃)amino)-2,7-naphthyridin-3-yl)-2-fluorocyclopropane-1-carboxamide (trans racemate)

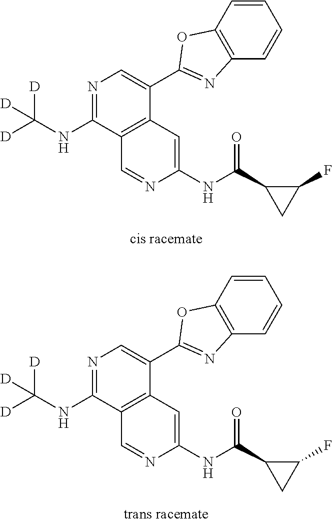 [SMILES: [2H]C([2H])([2H])Nc1ncc(-c2nc3ccccc3o2)c2cc(NC(=O)[C@@H]3C[C@@H]3F)ncc12 ; [2H]C([2H])([2H])Nc1ncc(-c2nc3ccccc3o2)c2cc(NC(=O)[C@@H]3C[C@H]3F)ncc12]

To a stirring mixture of 4-(benzo[d]oxazol-2-yl)-6-chloro-N-(methyl-d₃)-2,7-naphthyridin-1-amine (Example 431, step 2) (60.0 mg; 0.191 mmol; 1.00 eq.) in dioxane (3 mL) was added Pd₂(dba)₃ (17.5 mg; 0.019 mmol; 0.10 eq.), XantPhos (22.0 mg; 0.038 mmol; 0.20 eq.), Cs₂CO₃ (125.0 mg; 0.384 mmol; 2.01 eq.) and 2-fluorocyclopropane-1-carboxamide (40.0 mg; 0.388 mmol; 2.03 eq.) at room temperature. The reaction was stirred at 110° C. for 1 hour under nitrogen atmosphere. The resulting mixture was concentrated under vacuum. The residue was purified by flash chromatography on silica gel column using 2-10% of MeOH in CH₂Cl₂ as eluent to provide the mixture of cis and trans isomers. The mixture was purified by flash chromatography on pre-packed C18 column using 20-70% of MeCN in water (10 mmol/L NH₄HCO₃) as eluent to provide N-(5-(benzo[d]oxazol-2-yl)-8-((methyl-d₃)amino)-2,7-naphthyridin-3-yl)-2-fluorocyclopropane-1-carboxamide (cis racemate) as a yellow solid (17.1 mg, 23.5%) and N-(5-(benzo[d]oxazol-2-yl)-8-((methyl-d₃)amino)-2,7-naphthyridin-3-yl)-2-fluorocyclopropane-1-carboxamide (trans racemate) as a yellow solid (13.4 mg, 18.4%). LCMS (ESI) m/z 381.1, [M+H]⁺.

HNMR for Example 433: ¹H NMR (400 MHz, DMSO-d₆) δ 11.08 (s, 1H), 9.70 (s, 1H), 9.46 (s, 1H), 8.94 (s, 1H), 8.70-8.62 (m, 1H), 7.82-7.70 (m, 2H), 7.44-7.35 (m, 2H), 5.10-4.83 (m, 1H), 2.35-2.25 (m, 1H), 1.82-1.67 (m, 1H), 1.29-1.16 (m, 1H).

HNMR for Example 434: ¹H NMR (400 MHz, DMSO-d₆) δ 11.25 (s, 1H), 9.69 (s, 1H), 9.49 (s, 114), 8.96-8.82 (m, 2H), 7.81-7.71 (m, 2H), 7.45-7.36 (m, 2H), 5.11-4.85 (m, 1H), 2.72-2.58 (m, 1H), 1.65-1.50 (m, 1H), 1.41-1.28 (m, 1H).

### Example 435: Synthesis of (1S,2S)-2-fluoro-N-(5-(5-methoxybenzo[d]oxazol-2-yl)-8-((methyl-d₃)amino)-2,7-naphthyridin-3-yl)cyclopropane-1-carboxamide

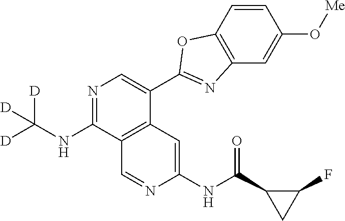 [SMILES: [2H]C([2H])([2H])Nc1ncc(-c2nc3cc(OC)ccc3o2)c2cc(NC(=O)[C@@H]3C[C@@H]3F)ncc12]

### Step 1: N-(5-(5-methoxybenzo[d]oxazol-2-yl)-8-((methyl-amino)-2,7-naphthyridin-3-yl)cyclopropanecarboxamide

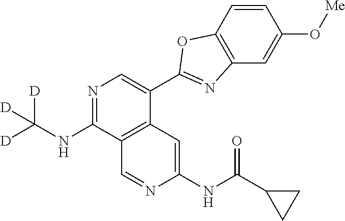 [SMILES: [2H]C([2H])([2H])Nc1ncc(-c2nc3cc(OC)ccc3o2)c2cc(NC(=O)C3CC3)ncc12]

To a stirring mixture of 5-methoxybenzo[d]oxazole (276.0 mg; 1.850 mmol; 1.20 eq.) and N-(5-bromo-8-((methyl-d₃)amino)-2,7-naphthyridin-3-yl)cyclopropanecarboxamide (500.1 mg; 1.542 mmol; 1.00 eq.) in DMF (5 mL) were added Pd(PPh₃)₄ (178.2 mg; 0.154 mmol; 0.10 eq.) and Cs₂CO₃ (1.51 g; 4.626 mmol; 3.00 eq.). The resulting solution was stirred at 110° C. for 4 hours under nitrogen atmosphere. The desired product was detected via. LCMS. The resulting mixture was diluted with EtOAc (30 mL) and washed with brine (3×5 mL). The organic layer was dried over anhydrous Na₂SO₄. After filtration, the filtrate was concentrated under reduced pressure. The residue was purified by flash chromatography on silica gel column using 0-10% of MeOH in CH₂Cl₂ as eluent to provide N-(5-(5-methoxybenzo[d]oxazol-2-yl)-8-((methyl-d₃)amino)-2,7-naphthyridin-3-yl)cyclopropanecarboxamide as a yellow solid (570.1 mg, 94.1%). LCMS (ESI) m/z 393.2, [M+H]⁺.

### Step 2: 4-(5-methoxybenzo[d]oxazol-2-yl)-N¹-(methyl-d₃)-2,7-naphthyridine-1,6-diamine

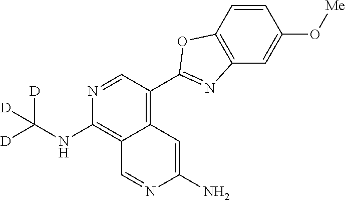 [SMILES: [2H]C([2H])([2H])Nc1ncc(-c2nc3cc(OC)ccc3o2)c2cc(N)ncc12]

To a stirring solution of N-(5-(5-methoxybenzo[d]oxazol-2-yl)-8-((methyl-d₃)amino)-2,7-naphthyridin-3-yl)cyclopropanecarboxamide (570.1 mg; 1.452 mmol; 1.00 eq.) in DMSO/water/MeOH (1:1.5:5, 22.5 mL) was-NaOH (580.9 mg; 14.525 mmol; 10.00 eq.). The resulting mixture was stirred at 60° C. overnight. The desired product was detected via LCMS. The resulting mixture was concentrated under reduced pressure to remove MeOH. The solids were collected by filtration and washed with water (3×5 mL) to provide 4-(5-methoxybenzo[d]oxazol-2-yl)-N¹-(methyl-d₃)-2,7-naphthyridine-1,6-diamine as a yellow solid (410.1 mg, 87.0%), LCMS (ESI) m/z 325.1, [M+H]⁺.

### Step 3: (1S,2S)-2-fluoro-N-(5-(5-methoxybenzo[d]oxazol-2-yl)-8-((methyl-d₃)amino)-2,7-naphthyridin-3-yl)cyclopropane-1-carboxamide

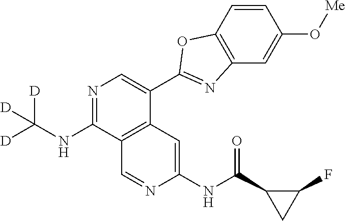 [SMILES: [2H]C([2H])([2H])Nc1ncc(-c2nc3cc(OC)ccc3o2)c2cc(NC(=O)[C@@H]3C[C@@H]3F)ncc12]

To a stirred mixture of 4-(5-methoxybenzo[d]oxazol-2-yl)-N¹-methyl-d₃)-2,7-naphthyridine-1,6-diamine (100.1 mg; 0.308 mmol; 1.00 eq.) and (1S,2S)-2-fluorocyclopropane-1-carboxylic acid (28.8 mg; 0.277 mmol; 0.90 eq.) in pyridine (4 mL) was added POCl₃ (141.8 mg; 0.924 mmol; 3.00 eq.) dropwise at 0° C., under nitrogen atmosphere. The resulting mixture was stirred at room temperature for 1 hour under nitrogen atmosphere. The reaction was quenched with water (10 mL) at 0° C. and extracted with EtOAc (3×20 mL). The combined organic layers were washed with brine (2×10 mL), dried over anhydrous Na₂SO₄. After filtration, the filtrate was concentrated under reduced pressure. The residue was purified by flash chromatography on silica gel column using 0-20% of MeOH in CH₂Cl₂ as eluent to afford (1S,2S)-2-fluoro-N-(5-(5-methoxybenzo[d]oxazol-2-yl)-8-((methyl-d₃)amino)-2,7-naphthyridin-3-yl)cyclopropane-1-carboxamide as a yellow solid (27.6 mg, 20.8%). LCMS (ESI) m/z 411.2, [M+H]⁺. ¹H NMR (400 MHz, DMSO-d₆) 11.32 (s, 1H), 9.73 (s, 1H), 9.64 (s, 1H), 8.64 (s, 1H), 7.69 (d, 8.8 Hz, 1H), 7.34 (d, J=2.4 Hz, 1H), 7.09-6.95 (m, 1H), 5.13-4.90 (m, 1H), 3.87 (s, 3H), 2.36-2.27 (m, 1H), 1.90-1.62 (m, 1H), 1.37-1.14 (m, 1H).

### Examples 436, 437, and 438

Synthesis of N-(5-(5-methoxybenzo[d]oxazol-2-yl)-8-((methyl-d₃)amino)-2,7-naphthyridin-3-yl)-2-methylcyclopropane-4-carboxamide (cis racemate) (Example 436), (1R,2S)—N-(5-((5-methoxybenzo[d]oxazol-2-yl)-8-((methyl-d₃)amino)-2,7-naphthyridin-3-yl)-2-methylcyclopropane-1-carboxamide (Example 437) and (1S,2R)—N-(5-(5-methoxybenzo[d]oxazol-2-3H)-8-((methyl-d₃)amino)-2,7-naphthyridin-3-yl)-2-methylcyclopropane-1-carboxamide (Example 438)

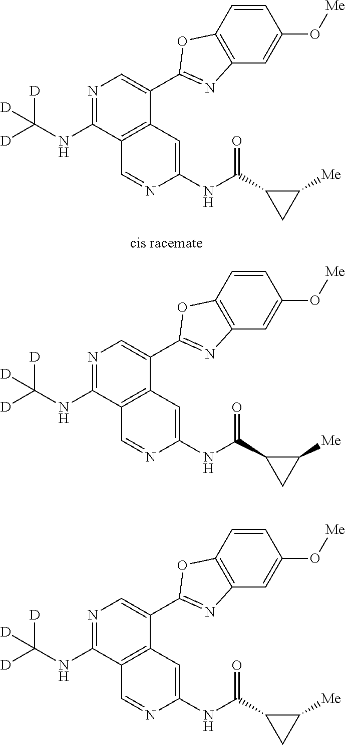 [SMILES: [2H]C([2H])([2H])Nc1ncc(-c2nc3cc(OC)ccc3o2)c2cc(NC(=O)[C@H]3C[C@H]3C)ncc12 ; [2H]C([2H])([2H])Nc1ncc(-c2nc3cc(OC)ccc3o2)c2cc(NC(=O)[C@@H]3C[C@@H]3C)ncc12 ; [2H]C([2H])([2H])Nc1ncc(-c2nc3cc(OC)ccc3o2)c2cc(NC(=O)[C@H]3C[C@H]3C)ncc12]

### Step 1: N-(5-(5-methoxybenzo[d]oxazol-2-yl)-8-((methyl-d₃)amino)-2,7-naphthyridin-3-yl)-2-methylcyclopropane-1-carboxamide (cis racemate)

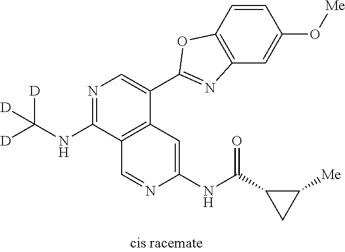 [SMILES: [2H]C([2H])([2H])Nc1ncc(-c2nc3cc(OC)ccc3o2)c2cc(NC(=O)[C@H]3C[C@H]3C)ncc12]

N-(5-(5-methoxybenzo[d]oxazol-2-yl)-8-((methyl-d₃)amino)-2,7-naphthyridin-3-yl)-2-methylcyclopropane-1-carboxamide (cis racemate) was synthesized using a similar procedure that was previously described in Example 435, by using 4-(5-methoxybenzo[d]oxazol-2-yl)-8-(methyl-d₃)-2,7-naphthyridine-1,6-diamine and 2-methylcyclopropane-1-carboxylic acid (cis racemate) as the starting material. LCMS (ESI) m/z 407.2, [M+H]⁺. NMR (400 MHz, DMSO-d₆) δ 10.90 (s, 1H), 9.62 (s, 1H), 9.43 (s, 1H), 8.90 (s, 1H), 8.70-8.47 (m, 1H), 7.62 (d, J=8.8 Hz, 1H), 7.29 (d, J=2.0 Hz, 1H), 7.05-6.88 (m, 1H), 3.86 (s, 3H), 2.15-2.04 (m, 1H), 1.38-1.29 (m, 1H), 1.17 (d, J=6.4 Hz, 3H), 1.06-0.98 (m, 1H), 0.92-0.87 (m, 1H).

### Step 2: (1R,2S)—N-(5-(5-methoxybenzo[d]oxazol-2-yl)-8-((methyl-d₃)amino)-2,7-naphthyridin-3-yl)-2-methylcyclopropane-1-carboxamide (Example 437) and (1S,2R)—N-(5-(5-methoxybenzo[d]oxazol-2-yl)-8-((methyl-d₃)amino)-2,7-naphthyridin-3-yl)-2-methylcyclopropane-1-carboxamide (Example 438)

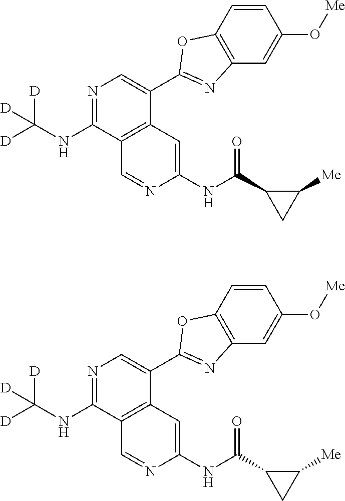 [SMILES: [2H]C([2H])([2H])Nc1ncc(-c2nc3cc(OC)ccc3o2)c2cc(NC(=O)[C@@H]3C[C@@H]3C)ncc12 ; [2H]C([2H])([2H])Nc1ncc(-c2nc3cc(OC)ccc3o2)c2cc(NC(=O)[C@H]3C[C@H]3C)ncc12]

N-(5-(5-methoxybenzo[d]oxazol-2-yl)-8-((methyl-d₃)amino)-2,7-naphthyridin-3-yl)-2-methylcyclopropane-1-carboxamide (cis racemate) was separated by Chiral-HPLC (Column: Chiral ART Cellulose-SA, 2×25 cm, 5 urn; Mobile Phase A: Hex (0.5% 2 M NH₃-MeOH), Mobile Phase B: IPA: CH₂Cl₂=1:1; Flow rate: 20 mL/min; Gradient: 30% B to 30% B in 30 min; Wave Length: 220/254 nm; R_{T1}(min): 19.86; R_{T2}(min): 27.22) to afford (1R,2S)—N-(5-(5-methoxybenzo[d]oxazol-2-yl)-8-((methyl-d₃)amino)-2,7-naphthyridin-3-yl)-2-methylcyclopropane-1-carboxamide (Example 437, the faster peak) as a yellow solid (18.8 mg, 10.7%) and (1S,2R)—N-(5-(5-methoxybenzo[d]oxazol-2-yl)-8-((methyl-d₃)amino)-2,7-naphthyridin-3-yl)-2-methylcyclopropane-1-carboxamide (Example 438, the slower peak) as a yellow solid (17.8 mg, 10.1%). The two configurations are arbitrarily assigned. LCMS (ESI) m/z 407.2, [M+H]⁺.

HNMR for Example 437: ¹H NMR (400 MHz, DMSO-d₆) δ 10.90 (s, 1H), 9.61 (s, 1H), 9.43 (s, 1H), 8.89 (s, 1H), 8.70-8.48 (m, 1H), 7.62 (d, J=8.8 Hz, 1H), 7.29 (d, 2.4 Hz, 1H), 7.08-6.89 (m, 1H), 3.86 (s, 3H), 2.19-2.05 (m, 1H), 1.39-1.28 (m, 1H), 1.17 (d, J=6.4 Hz, 3H), 1.09-0.96 (m, 1H), 0.95-0.80 (m, 1H).

HNMR for Example 438: ¹H NMR (400 MHz, DMSO-d₆) δ 10.90 (s, 1H), 9.61 (s, 1H), 9.43 (s, 1H), 8.89 (s, 1H), 8.70-8.48 (m, 1H), 7.62 (d, J=8.8 Hz, 1H), 7.29 (d, J=2.4 Hz, 1H), 7.08-6.89 (m, 1H), 3.86 (s, 3H), 2.19-2.05 (m, 1H), 1.39-1.28 (m, 1H), 1.17 (d, J===6.4 Hz, 3H), 1.09-0.96 (m, 1H), 0.95-0.80 (m, 1H).

### Example 439

Synthesis of (1S,2R)—N-(5-(5-(methoxy-d)benzo[d]oxazol-2-yl)-8-((methyl-d₃)amino)-2,7-naphthyridin-3-yl)-2-methylcyclopropane-1-carboxamide

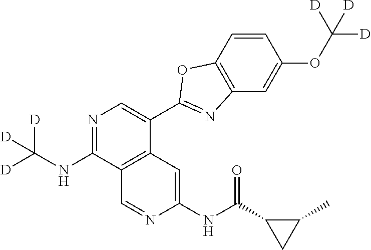 [SMILES: [2H]C([2H])([2H])Nc1ncc(-c2nc3cc(OC([2H])([2H])[2H])ccc3o2)c2cc(NC(=O)[C@H]3C[C@H]3C)ncc12]

### Step 1: 4-(5-(methoxy-d₃)benzo[d]oxazol-2-yl)-N¹-(methyl-d₃)-2,7-naphthyridine-1,6-diamine

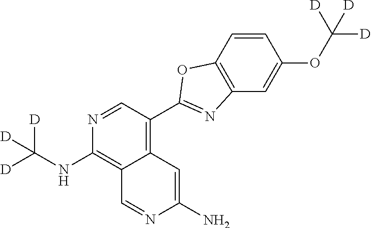 [SMILES: [2H]C([2H])([2H])Nc1ncc(-c2nc3cc(OC([2H])([2H])[2H])ccc3o2)c2cc(N)ncc12]

4-(5-(methoxy-d₃)benzo[d]oxazol-2-yl)-N¹-(methyl-d₃)-2,7-naphthyridine-1,6-diamine was synthesized using a similar procedure that was previously described in Example 435 by using N-(5-(5-(methoxy-d₃)benzo[d]oxazol-2-yl)-8-((methyl-d₃)amino)-2,7-naphthyridin-3-yl)cyclopropanecarboxamide (Example 365) as the starting material, LCMS (ESI) m/z 328.2 [M+H]⁺.

### Step 2: (1S,2R)—N-(5-(5-(methoxy-d₃)benzo[d]oxazol-2-yl)-8-((methyl-d₃)amino-2,7-naphthyridin-3-yl)-2-methylcyclopropane-1-carboxamide

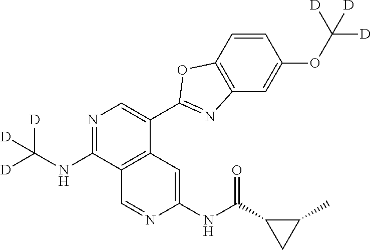 [SMILES: [2H]C([2H])([2H])Nc1ncc(-c2nc3cc(OC([2H])([2H])[2H])ccc3o2)c2cc(NC(=O)[C@H]3C[C@H]3C)ncc12]

(1S,2R)—N-(5-(5-(methoxy-d₃)benzo[d]oxazol-2-yl)-8-((methyl-d₃)amino)-2,7-naphthyridin-3-yl)-2-methylcyclopropane-1-carboxamide was synthesized using a similar procedure that was previously described in Example 435 by using 4-(5-(methoxy-d₃)benzo[d]oxazol-2-yl)-N¹-(methyl-d₃)-2,7-naphthyridine-1,6-diamine and (1S,2R)-2-methylcyclopropane-1-carboxylic acid as the starting material. LCMS (ESI) m/z 410.2 [M+H]⁺. ¹H NMR (400 MHz, DMSO-d₆) δ 11.21 (s, 1H), 10.29-9.89 (m, 1H), 9.87-9.79 (s, 1H), 9.79-9.61 (s, 1H), 8.61 (s, 1H), 7.69 (d, J=8.4 Hz, 1H), 7.33 (d, J=2.8 Hz, 1H), 7.09-7.00 (m, 1H), 2.19-2.09 (m, 1H), 1.40-1.30 (m, 1H), 1.16 (d, J=6.0 Hz, 3H), 1.10-1.00 (m, 1H), 0.95-0.89 (m, 1H).

### Example 440: Synthesis of (1S,2R)-2-methyl-N-(8-((methyl-d₃)amino)-5-(5-((S)-2-methylmorpholino)benzo[d]oxazol-2-yl)-2,7-naphthyridin-3-yl)cyclopropane-1-carboxamide)

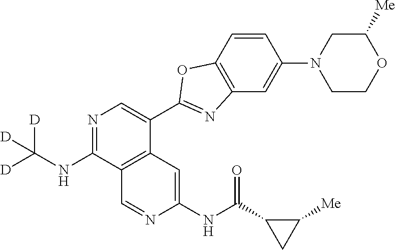 [SMILES: [2H]C([2H])([2H])Nc1ncc(-c2nc3cc(N4CCO[C@@H](C)C4)ccc3o2)c2cc(NC(=O)[C@H]3C[C@H]3C)ncc12]

### Step 1: (S)—N-(8-((methyl-d₃ amino)-5-(5-(2-methylmorpholino)benzo[d]oxazol-2-yl)-2,7-naphthyridin-3-yl)cyclopropanecarboxamide

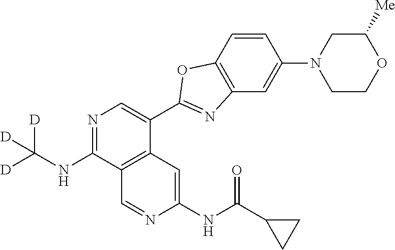 [SMILES: [2H]C([2H])([2H])Nc1ncc(-c2nc3cc(N4CCO[C@@H](C)C4)ccc3o2)c2cc(NC(=O)C3CC3)ncc12]

To a stirring mixture of (S)-5-(2-methylmorpholino)benzo[d]oxazole (800.0 mg, 3.684 mmol, 1.20 eq.) and N-(5-bromo-8-((methyl-d₃)amino)-2,7-naphthyridin-3-yl)cyclopropanecarboxamide (990.1 mg; 3.07 mmol; 1.00 eq.) in DMF (25 mL) were added Pd(PPh₃)₄ (354.3 mg; 0.307 mmol; 0.10 eq.) and Cs₂CO₃ (3.01 g; 9.21 mmol; 3.00 eq.) under nitrogen atmosphere. The resulting mixture was stirred at 110° C. for 12 hours. The desired product was detected via LCMS. The mixture was concentrated under reduced pressure. The residue was purified by flash chromatography on silica gel column using 5-10% of MeOH in CH₂Cl₂ to afford (S)—N-(8-((methyl-di)amino)-5-(5-(2-methylmorpholino)benzo[d]oxazol-2-yl)-2,7-naphthyridin-3-yl)cyclopropanecarboxamide as a yellow solid (1.2 g, 84.7%). LCMS (ESI) m/z 462.2, [M+H]⁺.

### Step 2: (S)—N¹(methyl-d₃)-4-(5-(2-methylmorpholino)benzo[d]oxazol-2-yl)-2,7-naphthyridine-1,6-diamine

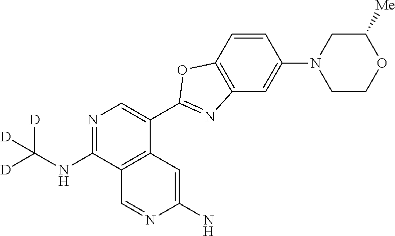 [SMILES: [2H]C([2H])([2H])Nc1ncc(-c2nc3cc(N4CCO[C@@H](C)C4)ccc3o2)c2cc(N)ncc12]

A mixture of (S)—N-(8-((methyl-d₃)amino)-5-(5-(2-methylmorpholino)benzo[d]oxazol-2-yl)-2,7-naphthyridin-3-yl)cyclopropanecarboxamide (1.2 g; 2.600 mmol, 1.00 eq.) in DMSO (6 mL). To the above solution were added MeOH (30 mL) and a solution of NaOH (1.2 g; 30.002 mmol; 11.54 eq.) in water (10 mL). The resulting mixture was stirred at 60° C. for 12 hours. The desired product was detected via LCMS. The mixture was concentrated under vacuum to remove MeOH. To the mixture was added water (200 mL) to precipitate solids. The solids were collected by filtration and washed with water to afford (S)—N¹-(methyl-d₃)-4-(5-(2-methylmorpholino)benzo[d]oxazol-2-yl)-2,7-naphthyridine-1,6-diamine as a yellow solid (0.92 g, 90.1%). LCMS (ESI) m/z 394.2, [M+H]⁺.

### Step 3: (1S,2R)-2-methyl-N-(8-((methyl-d₃)amino)-5-(5-((S)-2-methylmorpholino)benzo[d]oxazol-2-yl)-2,7-naphthyridin-3-yl)cyclopropane-1-carboxamide

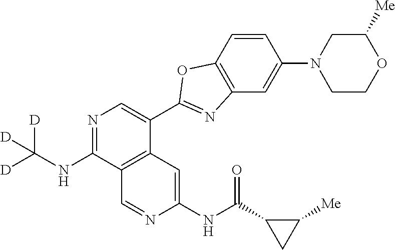 [SMILES: [2H]C([2H])([2H])Nc1ncc(-c2nc3cc(N4CCO[C@@H](C)C4)ccc3o2)c2cc(NC(=O)[C@H]3C[C@H]3C)ncc12]

A mixture of (S),N¹-(methyl-d₃)-4-(5-(2-methylmorpholino)benzo[d]oxazol-2-yl)-2,7-naphthyridine-1,6-diamine (500.0 mg; 1.271 mmol; 1.00 eq.) and (1S,2R)-2-methylcyclopropane-1-carboxylic acid (120.0 mg; 1.199 mmol; 0.94 eq.) in pyridine (10 mL) was added. POCl₃ (600 mg; 3.913 mmol; 3.08 eq.) dropwise at 0° C. under nitrogen atmosphere. The resulting solution was stirred at room temperature for 1 hour. The desired product was detected via LCMS. The reaction was quenched by a saturated NaHCO₃ aqueous solution (1 M in water, 30 mL), extracted with EtOAc/THF (3:1, 3×100 mL). The combined organic layer was dried over anhydrous sodium sulfate and concentrated under vacuum. The residue was purified by flash chromatography on silica gel column using 5-10% of Maki: in CH₂Cl₂ to afford a crude product. The crude product was purified by flash chromatography on pre-packed C18 column using 20-50% of THF/MeCN (1:3) in water (10 mmol/L, NH₄HCO₃) to provide (1S,2R)-2-methyl-N-(8-((methyl-d₃)amino)-5-(5-((S)-2-methylmorpholino)benzo[d]oxazol-2-yl)-2,7-naphthyridin-3-yl)cyclopropane-1-carboxamide as a yellow solid (307.8 mg, 50.9%). LCMS (ESI) m/z 476.2, [M+H]⁺. ¹H NMR (400 MHz, DMSO-d₆) δ 10.91 (s, 1H), 9.61 (s, 1H), 9.43 (s, 1H), 8.88 (s, 1H), 8.62-8.55 (m, 1H), 7.57 (d, J=8.8 Hz, 1H), 7.23 (d, J=2.4 Hz, 1H), 7.06 (dd, J=8.8, 2.4 Hz, 1H), 3.97-3.89 (m, 1H), 3.73-3.66 (m, 2H), 3.62-3.58 (m, 1H), 3.53-3.48 (m, 1H), 2.77-2.68 (m, 1H), 2.46-2.37 (m, 1H), 2.15-2.09 (m, 1H), 1.39-1.26 (m, 1H), 1.25-1.12 (m, 6H), 1.06-0.99 (m, 1H), 0.94-0.86 (m, 1H).

### Example 441: Synthesis of (1S,2R)-2-methyl-N-(8-((methyl-d₃)amino)-5-(5-((R)-2-methylmorpholino)benzo[d]oxazol-2-yl)-2,7-naphthyridin-3-yl)cyclopropane-1-carboxamide

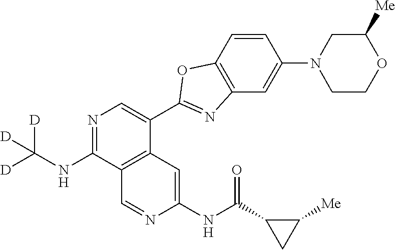 [SMILES: [2H]C([2H])([2H])Nc1ncc(-c2nc3cc(N4CCO[C@H](C)C4)ccc3o2)c2cc(NC(=O)[C@H]3C[C@H]3C)ncc12]

### Step 1: (R)—N-(8-((methyl-d₃)amino)-5-(5-(2-methylmorpholino)benzo[d]oxazol-2-yl)-2,7-naphthyridin-3-yl)cyclopropanecarboxamide

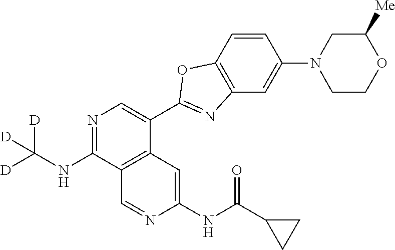 [SMILES: [2H]C([2H])([2H])Nc1ncc(-c2nc3cc(N4CCO[C@H](C)C4)ccc3o2)c2cc(NC(=O)C3CC3)ncc12]

(R)—N-(8-((methyl-d₃)amino)-5-(5-(2-methylmorpholino)benzo[d]oxazol-2-yl)-2,7-naphthyridin-3-yl)cyclopropanecarboxamide was synthesized using a similar procedure that was previously described in Example 440 by using N-(5-bromo-8-((methyl-d₃)amino)-2,7-naphthyridin-3-yl)cyclopropanecarboxamide and (R)-5-(2-methylmorpholino)benzo[d]oxazole as the starting material. LCMS (ESI) m/z 462.2, [M+H]⁺.

### Step 2: (R)—N¹-(methyl-d₃)-4-(5-(2-methylmorpholino)benzo[d]oxazol-2-yl)-2,7-naphthyridine-1,6-diamine

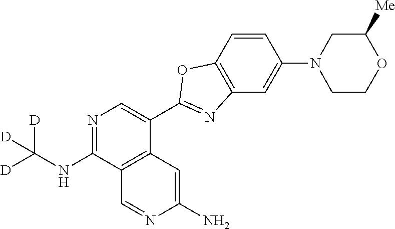 [SMILES: [2H]C([2H])([2H])Nc1ncc(-c2nc3cc(N4CCO[C@H](C)C4)ccc3o2)c2cc(N)ncc12]

(R)—N¹-(methyl-d₃)-4-(5-(2-methylmorpholino)benzo[d]oxazol-2-yl)-2,7-naphthyridine-1,6-diamine was synthesized using a similar procedure that was previously described in Example 440 by using (R)—N-(8-((methyl-d₃)amino)-5-(5-(2-methylmorpholino)benzo[d]0 oxazol-2-yl)-2,7-naphthyridin-3-yl)cyclopropanecarboxamide as the starting material. LCMS (ESI) m/z 394.2, [M+H]⁺.

### Step 3: (1S,2S)-2-methyl-N-(8-((methyl-d₃)amino)-5-(5-((R)-2-methylmorpholino)benzo[d]oxazol-2-yl)-2,7-naphthyridin-3-yl)cyclopropane-1-carboxamide

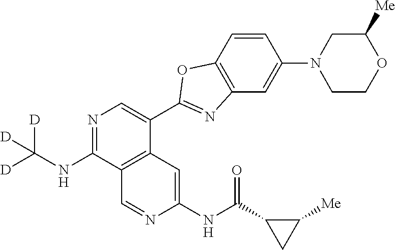 [SMILES: [2H]C([2H])([2H])Nc1ncc(-c2nc3cc(N4CCO[C@H](C)C4)ccc3o2)c2cc(NC(=O)[C@H]3C[C@H]3C)ncc12]

(1S,2R)-2-methyl-N-(8-((methyl-d₃)amino)-5-(5-((R)-2-methylmorpholino)benzo[d]oxazol-2-yl)-2,7-naphthyridin-3-yl)cyclopropane-1-carboxamide was synthesized using a similar procedure that was previously described in Example 440 by using (R)—N¹-(methyl-d₃)-4-(5-(2-methylmorpholino)benzo[d]oxazol-2-yl)-2,7-naphthyridine-1,6-diamine and (1S,2R)-2-methylcyclopropane-1-carboxylic acid as the starting material. LCMS (ESI) m/z 476.2, [M+H]⁺. ¹H NMR (400 MHz, DMSO-d₆) 10.89 (s, 1H), 9.60 (s, 1H), 9.43 (s, 1H), 8.88 (s, 1H), 8.66-8.49 (m, 1H), 7.57 (d, 8.8 Hz, 1H), 7.23 (d, J=2.0 Hz, 1H), 7.13-6.96 (m, 1H), 3.98-3.90 (m, 1H), 3.74-3.66 (m, 2H), 3.62 (d, J=11.2 Hz, 1H), 3.55-3.49 (m, 1H), 2.76-2.68 (m, 1H), 2.44-2.36 (m, 1H), 2.18-2.08 (m, 1H), 1.37-1.29 (m, 1H), 1.24-1.14 (m, 6H), 1.08-1.00 (m, 1H), 0.96-0.81 (m, 1H).

### Example 442: Synthesis of 1-fluoro-N-(8-((methyl-d₃)amino)-5-(5-morpholinobenzo[d]oxazol-2-yl)-2,7-naphthyridin-3-yl)cyclopropane-1-carboxamide

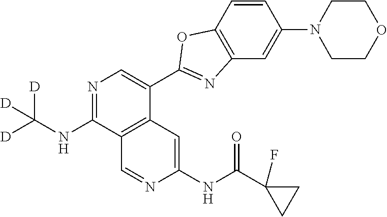 [SMILES: [2H]C([2H])([2H])Nc1ncc(-c2nc3cc(N4CCOCC4)ccc3o2)c2cc(NC(=O)C3(F)CC3)ncc12]

### Step 1: N¹-(methyl-d₃)-4-(5-morpholinobenzo[d]oxazol-2-yl)-2,7-naphthyridine-1,6-diamine

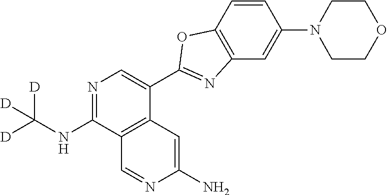 [SMILES: [2H]C([2H])([2H])Nc1ncc(-c2nc3cc(N4CCOCC4)ccc3o2)c2cc(N)ncc12]

N¹-(methyl-d₃)-4-(5-morpholinobenzo[d]oxazol-2-yl)-2,7-naphthyridine-1,6-diamine was synthesized using a similar procedure that was previously described in Example 440 by using (8-((methyl-d₃)amino)-5-(5-morpholinobenzo[d]oxazol-2-yl)-2,7-naphthyridin-3-yl)cyclopropanecarboxamide (Example 364) as the starting material. LCMS (ESI) m/z 380.2, [M+H]⁺.

### Step 2: 1-fluoro-N-(8-((methyl-d₃)amino)-5-(5-morpholinobenzo[d]oxazol-2-yl)-2,7-naphthyridin-3-yl)cyclopropane-1-carboxamide

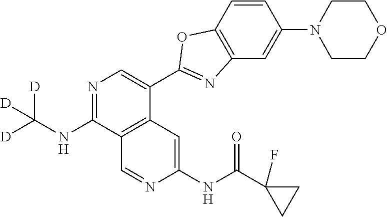 [SMILES: [2H]C([2H])([2H])Nc1ncc(-c2nc3cc(N4CCOCC4)ccc3o2)c2cc(NC(=O)C3(F)CC3)ncc12]

1-fluoro-N-(8-((methyl-d₃)amino)-5-(5-morpholinobenzo[d]oxazol-2-yl)-2,7-naphthyridin-3-yl)cyclopropane-1-carboxamide was synthesized using a similar procedure that was previously described in example 440, by using N¹-methyl-d₃)-4-(5-morpholinobenzo[d]oxazol-2-yl)-2,7-naphthyridine-1,6-diamine and 1-fluorocyclopropane-1-carboxylic acid as the starting material. LCMS (ESI) m/z 466.2, [M+H]⁺. ¹HNMR (400 MHz, DMSO-d₆) δ 10.39 (s, 1H), 9.68 (s, 1H), 9.49 (s, 1H), 8.93 (s, 1H), 8.77-8.28 (s, 1H), 7.59 (d, 8.8 Hz, 1H), 7.24 (d, J=2.0 Hz, 1H), 7.15-6.89 (m, 1H), 3.88-3.68 (m, 4H), 3.21-3.06 (m, 4H), 1.58-1.39 (m, 4H).

### Example 443: Synthesis of (1S,2R)-2-methyl-N-(8-((methyl-d₃)amino)-5-(5-morpholinobenzo[d]oxazol-2-yl)-2,7-naphthyridin-3-yl)cyclopropane-1-carboxamide

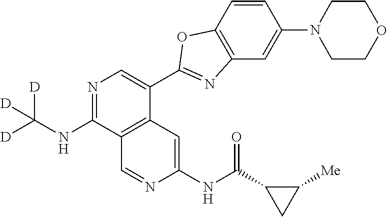 [SMILES: [2H]C([2H])([2H])Nc1ncc(-c2nc3cc(N4CCOCC4)ccc3o2)c2cc(NC(=O)[C@H]3C[C@H]3C)ncc12]

(1S,2R)-2-methyl-N-(8-((methyl-d)amino)-5-(5-morpholinobenzo[d]oxazol-2-yl)-2,7-naphthyridin-3-yl)cyclopropane-1-carboxamide was synthesized using a similar procedure that was previously described in Example 440 by using N¹-methyl-d₃)-4-(5-morpholinobenzo[d]oxazol-2-yl)-2,7-naphthyridine-1,6-diamine and (1S,2R)-2-methylcyclopropane-1-carboxylic acid as the starting material. LCMS (ESI) m/z 462.2, [M+H]⁺. ¹H NMR (400 MHz, DMSO-d₆) δ 10.91 (s, 1H), 9.61 (s, 1H), 9.43 (s, 1H), 8.88 (s, 1H), 8.69-8.48 (m, 1H), 7.58 (d, J=9.2 Hz, 1H), 7.24 (d, J=2.4 Hz, 1H), 7.13-6.98 (m, 1H), 3.89-3.70 (m, 4H), 3.23-3.08 (m, 4H), 2.15-2.09 (m, 1H), 1.41-1.22 (m, 1H), 1.17 (d, J=6.4 Hz, 3H), 1.10-0.99 (m, 1H), 0.99-0.81 (m, 1H).

### Example 444: Synthesis of N-(5-(4-acetamidobenzo[d]oxazol-2-yl)-8-(methylamino)-2,7-naphthyridin-3-yl)cyclopropanecarboxamide

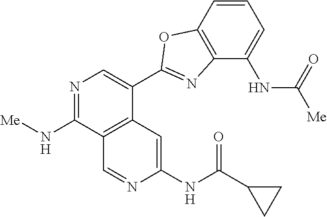 [SMILES: CNc1ncc(-c2nc3c(NC(C)=O)cccc3o2)c2cc(NC(=O)C3CC3)ncc12]

A solution of N-(5-(4-bromobenzo[d]oxazol-2-yl)-8-(methylamino)-2,7-naphthyridin-3-yl)cyclopropanecarboxamide (Example 272) (50.0 mg; 0.114 mmol; 1.00 eq), EPhos Pd G₄ (10.4 mg; 0.011 mmol, 0.10 eq), EPhos (6.1 mg; 0.011 mmol; 0.10 eq), Cs₂CO₃ (148.7 mg; 0.456 mmol; 4.00 eq) and acetamide (20.2 mg; 0.342=mmol; 3.00 eq) in 1,4-dioxane (3 mL) was stirred at 120° C. overnight under nitrogen atmosphere. The desired product was detected via LCMS. The resulting mixture was concentrated under reduced pressure. The residue was purified by flash chromatography on silica gel column using 2-10% of MeOH in CH₂Cl₂ to afford the crude product. The crude product was re-crystallized with a mixture of MeOH/CH₂Cl₂ (1:7, 10 mL) to afford N-(5-(4-acetamidobenzo[d]oxazol-2-yl)-8-(methylamino)-2,7-naphthyridin-3-yl)cyclopropanecarboxamide as a yellow solid (15.5 mg, 32.4%). LCMS (ESI) m/z 417.1, [M+H]⁺. ¹H NMR (400 MHz, DMSO-d₆) δ 11.14 (s, 1H), 10.20 (s, 1H), 9.45 (s, 1H), 9.26 (5, 1H), 8.91 (5, 1H), 8.75-8.64 (m, 1H), 7.98 (d, J=8.0 Hz, 1H), 7.44 (d, J=4.4 Hz, 1H), 7.37-7.29 (m, 1H), 3.09 (d, J=4.4 Hz, 3H), 2.26 (s, 3H), 2.18-2.09 (m, 1H), 0.94-0.84 (m, 4H).

### Example 445: Synthesis of N-(5-(5-(1H-1,2,4-triazol-3-yl)benzo)-8-(methylamino)-2,7-naphthyridin-3-yl)cyclopropanecarboxamide

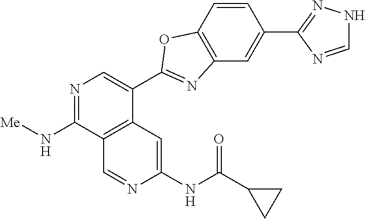 [SMILES: CNc1ncc(-c2nc3cc(-c4nc[nH]n4)ccc3o2)c2cc(NC(=O)C3CC3)ncc12]

A mixture of 2-(6-(cyclopropanecarboxamido)-1-(methylamino)-2,7-naphthyridin-4-benzo[d]oxazole-5-carboxamide (Example 237) (30 mg; 0.075 mmol; 1.00 eq.) and DMF-DMA (88.8 mg; 0.750 mmol; 10.00 eq.) was stirred at 95° C. for 1 hour. The resulting mixture was concentrated under reduced pressure. This crude mixture was re-dissolved in EtOH (1 mL) which was named ‘solution A’. Another solution of AcOH (1 mL) in EtOH (4 mL) was allowed to cool down to −10° C., to this mixture was added NH₂NH₂·H₂O (80%) (37.3 mg; 0.750 mmol; 10.00 eq.) and ‘solution A’ dropwise at −10° C. The resulting mixture was stirred at room temperature overnight. The desired product was detected via LCMS. The solvent was concentrated under reduced pressure. The residue was purified by flash chromatography on pre-packed C18 column using 20-70% of MeCN in water (10 mmol/L NH₄HCO₃) as eluent to provide N-(5-(5-(1H-1,2,4-triazol-3-yl)benzo[d]oxazol-2-yl)-8-(methylamino)-2,7-naphthyridin-3-yl)cyclopropanecarboxamide (5.2 mg, 16.0%). LCMS (ESI) m/z 427.2, [M+H]⁺. ¹H NMR (400 MHz, DMSO-d₆) 14.16 (s, 1H), 11.07 (s, 1H), 9.80 (s, 1H), 9.46 (s, 1H), 8.96 (s, 1H), 8.81-8.60 (m, 2H), 8.43-8.29 (m, 1H), 8.11-8.02 (m, 1H), 7.96-7.72 (m, 1H), 3.11 (d, J=4.4 Hz, 3H), 2.19-2.02 (m, 1H), 0.98-0.85 (m, 4H).

### Example 446: Synthesis of N-(5-(5-(2-methyl-5-oxo-2,5-dihydro-1H-pyrazol-1-yl)benzo[d]oxazol-2-yl)-8-(methylamino)-2,7-naphthyridin-3-yl)cyclopropanecarboxamide

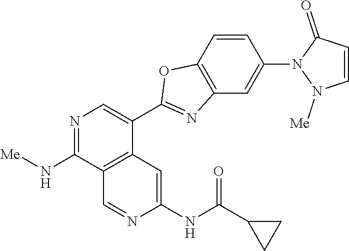 [SMILES: CNc1ncc(-c2nc3cc(-n4c(=O)ccn4C)ccc3o2)c2cc(NC(=O)C3CC3)ncc12]

### Step 1: 1-methyl-2-(2-methylbenzo[d]oxazol-5-yl)-1,2-dihydro-3H-pyrazol-3-one

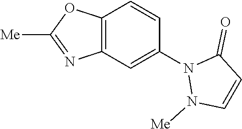 [SMILES: Cc1nc2cc(-n3c(=O)ccn3C)ccc2o1]

To a stirred solution of 5-bromo-2-methylbenzo[d]oxazole (200.0 mg; 0.943 mmol; 1.00 eq.) and 1-methyl-1,2-dihydro-3H-pyrazol-3-one (92.5 mg; 0.943 mmol; 1.00 eq.) in DMF (2 mL) were added N¹,N²-dimethylcyclohexane-1,2-diamine (trans racemate) (26.8 trig; 0.189 mmol; 0.20 eq.), CuI (17.9 mg; 0.094 mmol; 0.10 eq.), K₂CO₃ (391.0 mg; 2.829 mmol; 3.00 eq.). The resulting mixture was stirred at 120° C. for 1 hour under nitrogen atmosphere. The desired product was detected via LCMS. The mixture was allowed to cool down to room temperature. The mixture was purified by flash chromatography on pre-packed C18 column using 20-60% of MeOH in water (10 mmol/L NH₄HCO₃) as eluent to provide 1-methyl-2-(2-methylbenzo[d]oxazol-5-yl)-1,2-dihydro-3H-pyrazol-3-one as a white solid (64.9 mg, 30.1%). LCMS (ESI) m/z 230.1, [M+H]⁺.

### Step 2: 2-(3-amino-4-hydroxyphenyl)-1-methyl-1,2-dihydro-3H-pyrazol-3-one

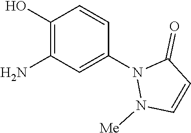 [SMILES: Cn1ccc(=O)n1-c1ccc(O)c(N)c1]

To a solution of 1-methyl-2-(2-methylbenzo[d]oxazol-5-yl)-1,2-dihydro-3H-pyrazol-3-one (60.0 mg; 0.262 mmol; 1.00 eq.) in EtOH (1 mL) was added a solution of HCl (4N, 1 mL). The mixture was stirred at 100° C. for 4 hours under nitrogen atmosphere. The desired product was detected via LCMS. The mixture was allowed to cool down to room temperature and concentrated under reduced pressure to afford 2-(3-amino-4-hydroxyphenyl)-1-methyl-1,2-dihydro-3/1-pyrazol-3-one as a brown solid (40.0 mg, 74.5%). LCMS (ESI) m/z 206.1, [M+H]⁺.

### Step 3: 2-(benzo[d]oxazol-5-yl)-1-methyl-1,2-dihydro-3H-pyrazol-3-one

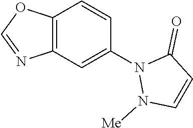 [SMILES: Cn1ccc(=O)n1-c1ccc2ocnc2c1]

A solution of 2-(3-amino-4-hydroxyphenyl)-1-methyl-1,2-dihydro-3H-pyrazol-3-one (40.0 mg; 0.195 mmol; 1.00 eq.) in trimethoxymethane (3 mL) was stirred at 100° C. overnight under nitrogen atmosphere. LC-NIS indicated that the starting material was consumed. The mixture was allowed to cool down to room temperature and the resulting mixture was concentrated under reduced pressure. The residue was purified by flash chromatography on silica gel column using 10-30% of EtOAc in petroleum ether as eluent to provide 2-(benzo[d]oxazol-5-yl)-1-methyl-1,2-dihydro-3H-pyrazol-3-one as a brown solid (40.2 mg, 95.4%). LCMS (ESI) m/z 216.1, [M+H]⁺.

### Step 4: N-(5-(5-(2-methyl-5-oxo-2,5-dihydro-1H-pyrazol-1-yl(benzo[d]oxazol-2-yl)-8-(methylamino)-2,7-naphthyridin-3-yl)cyclopropanecarboxamide

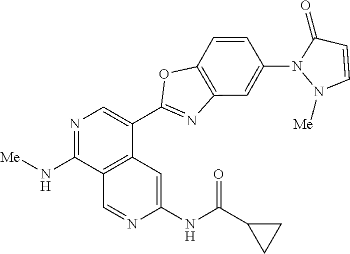 [SMILES: CNc1ncc(-c2nc3cc(-n4c(=O)ccn4C)ccc3o2)c2cc(NC(=O)C3CC3)ncc12]

To a stirred solution of 2-(benzo[d]oxazol-5-yl)-1-methyl-1,2-dihydro-3H-pyrazol-3-one (40.2 mg; 0.187 mmol; 1.20 eq.) and N-(5-bromo-8-(methylamino)-2,7-naphthyridin-3-yl)cyclopropanecarboxamide (50.0 mg; 0.156 mmol; 1.00 eq.) in DMF (1 mL) were added Pd(PPh₃)₄ (18.0 mg; 0.016 mmol; 0.10 eq.) and Cs₂CO₃ (152.2 mg; 0.468 mmol; 3.00 eq.) at room temperature. The resulting mixture was stirred at 110° C. overnight under nitrogen atmosphere. The desired product was detected via LCMS. The mixture was allowed to cool down to room temperature and concentrated under reduced pressure. The residue was purified by flash chromatography on silica gel column using 5-8% of MeOH in CH₂Cl₂ as eluent to provide N-(5-(5-(2-methyl-5-oxo-2,5-dihydro-1H-pyrazol-1-yl)benzo[d]oxazol-2-yl)-8-(methylamino)-2,7-naphthyridin-3-yl)cyclopropanecarboxamide as a brown solid (13.9 mg, 19.5%). LCMS (ESI) 456.2, [M+H]⁺. ¹H NMR (400 MHz; DMSO-d₆) δ 11.06 (s, 1H), 9.72. (s, 1H), 9.45 (s, 1H), 8.95 (s, 1H), 8.73-8.68 (m, 1H), 7.96 (d, J=3.6 Hz, 1H), 7.85 (d, J=8.8 Hz, 1H), 7.67 (d, J=2.0 Hz, 1H), 7.31 (dd, J=8.8, 2.0 Hz, 1H), 5.45 (d, J=3.6 Hz, 1H), 3.20 (s, 3H), 3.10 (d, J=4.4 Hz, 3H), 2.16-2.05 (m, 1H), 0.97-0.81 (m, 4H).

### Example 447: Synthesis of N-(5-(5-(9-methyl-3-oxa-7,9-diazabicyclo[3.3.1]nonan-7-yl)benzo[d]oxazol-2-yl)-8-(methylamino)-2,7-naphthyridin-3-yl)cyclopropanecarboxamide

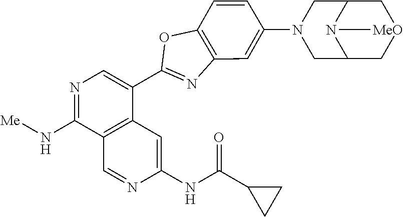

To a stirred mixture of Cs₂CO₃ (500.0 mg; 1.535 mmol; 6.73 eq.), EPhos (30.0 mg; 0.056 mmol; 0.25 eq.) and EPhos Pd G₄ (45.0 mg; 0.049 mmol; 0.21 eq.) in 1,4-dioxane (10 mL) were added N-(5-(5-bromobenzo[d]oxazol-2-yl)-8-(methylamino)-2,7-naphthyridin-3-yl)cyclopropanecarboxamide (100.0 mg; 0.228 mmol; 1.00 eq.) and 9-methyl-3-oxa-7,9-diazabicyclo[3.3.1]nonane hydrochloride (120.0 mg; 0.844 mmol; 3.70 eq.) under nitrogen atmosphere. The resulting solution was stirred at 100° C. for 48 hours. The desired product was detected via. LCMS. The reaction mixture was concentrated under vacuum. The residue was purified by flash chromatography on silica gel column using 2-10% of MeOH in CH₂Cl₂ as eluent to afford the crude product. The crude product was purified by flash chromatography on pre-packed C18 column using 50-70% of THF/ACN (1:3) in water (10 mmol/L, NH₄HCO₃) as eluent to provide N-(5-(5-(9-methyl-3-oxa-7,9-diazabicyclo[3.3.1]nonan-7-yl)benzo[d]oxazol-2-yl)-8-(methylamino)-2,7-naphthyridin-3-yl)cyclopropanecarboxamide as a yellow solid (13.4 mg, 11.8%). LCMS (ESI) m/z 500.2, [M+H]⁺. ¹H NMR (400 MHz, DMSO-d₆) δ 11.03 (s, 1H), 9.69 (s, 1H), 9.44 (s, 1H), 8.89 (s, 1H), 8.69-8.55 (m, 1H), 7.56 (d, 9.2 Hz, 1H), 7.08 (d, J=2.4 Hz, 1H), 6.95 (dd, J=9.2, 2.4 Hz, 1H), 3.92-3.76 (m, 4H), 3.58-3.46 (m, 2H), 3.31-3.25 (m, 2H), 3.09 (d, 4.4 Hz, 3H), 2.87-2.79 (m, 2H), 2.49 (s, 3H), 2.16-2.07 (m, 1H), 0.99-0.82 (m, 4H).

### Example 448: Synthesis of N-(8-(methylamino)-5-(5-(tetrahydro-2H-pyran-4-yl)benzo[d]oxazol-2-yl)-2,7-naphthyridin-3-yl)cyclopropanecarboxamide

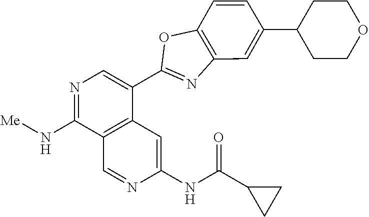 [SMILES: CNc1ncc(-c2nc3cc(C4CCOCC4)ccc3o2)c2cc(NC(=O)C3CC3)ncc12]

To a stirred solution of N-(5-(5-(3,6-dihydro-2H-pyran-4-yl)benzo[d]oxazol-2-yl)-8-(methylamino)-2,7-naphthyridin-3-yl)cyclopropanecarboxamide (Example 342) (80.0 mg; 0.181 mmol; 1.00 eq) in THF (20 mL) was added 10% Pd/C (40.0 mg; 100% w/w) under nitrogen atmosphere. The resulting mixture was hydrogenated at room temperature for 14 hours under hydrogen atmosphere (2 atm). The desired product was detected via LCMS. After filtration, the filtrate was concentrated under reduced pressure. The residue was purified by flash chromatography on silica gel column using 0-15% of MeOH in CH₂Cl₂ as eluent to afford the crude product. The crude product was dissolved in DMSO (3 mL) and added dropwise into water (30 mL), whereupon the precipitated product was collected via filtration and washed with water (3×5 mL) to provide N-(8-(methylamino)-5-(5-(tetrahydro-2H-pyran-4-yl)benzo[d]oxazol-2-yl)-2,7-naphthyridin-3-yl)cyclopropanecarboxamide as a light yellow solid (10.2 mg, 12.1%). LCMS (ESI) m/z 444.2, [M+H]⁺. ¹H NMR (400 MHz, DMSO-d₆) δ 11.04 (s, 1H), 9.68 (s, 1H), 9.44 (s, 1H), 8.90 (s, 1H), 8.71-8.40 (m, 1H), 7.71-7.51 (m, 2H), 7.39-7.22 (m, 1H), 4.09-3.83 (m, 2H), 3.56-3.40 (m, 2H), 3.09 (d, J=4.4 Hz, 3H), 3.01-2.89 (m, 1H), 2.17-2.04 (m, 1H), 1.90-1.68 (m, 4H), 0.93-0.82 (m, 4R).

### Example 449: Synthesis of N⁶-2,6-dimethylpyrimidin-4-yl)-N¹-methyl-4-(5-morpholinobenzo[d]oxazol-2-yl)-2,7-naphthyridine-1,6-diamine

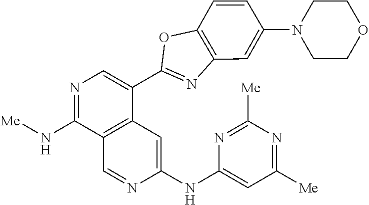 [SMILES: CNc1ncc(-c2nc3cc(N4CCOCC4)ccc3o2)c2cc(Nc3cc(C)nc(C)n3)ncc12]

To a stirred solution of N¹-methyl-4-(5-morpholinobenzo[d]oxazol-2-yl)-2,7-naphthyridine-1,6-diamine (80.0 mg; 0.213 mmol; 1.00 eq.) in DMA (3 mL) were added Pd₂(dba)₃ (23.4 mg; 0.026 mmol, 0.12 eq.), XantPhos (30.0 mg; 0.052 mmol, 0.24 eq.), Cs₂CO₃ (138.7 mg; 0.426 mmol; 2.00 eq.) and 4-chloro-2,6-dimethylpyrimidine (30.2 mg; 0.212 mmol; 1.00 eq.) under nitrogen atmosphere. The resulting solution was stirred at 130° C. for 2 hours under nitrogen atmosphere. The desired product was detected via LCMS. The mixture was purified by flash chromatography on pre-packed C18 column using 20-90% of MeOH in water (10 mmol/L NH₄HCO₃) as eluent to provide the crude product. The product was purified by flash chromatography on silica gel column using 2-10% of MeOH in CH₂Cl₂ as eluent to provide N⁶-(2,6-dimethylpyrimidin-4-yl)-N¹-methyl-4-(5-morpholinobenzo[d]oxazol-2-yl)-2,7-naphthyridine-1,6-diamine as a yellow solid (24.0 mg, 23.4%). LCMS (ESI) m/z 483.2, [M+H]⁺. ¹H NMR (400 MHz, DMSO-d₆) δ 10.59 (s, 1H), 9.94 (s, 1H), 9.43 (s, 1H), 8.88 (s, 1H), 8.69-8.44 (m, 1H), 7.62 (d, J=8.8 Hz, 1H), 7.25 (d, J=2.0 Hz, 1H), 7.19-6.75 (m, 2H), 3.84-3.77 (m, 4H), 3.19-3.12 (m, 4H), 3.10 (d, J=4.4 Hz, 3H), 2.66 (s, 3H), 2.38 (s, 3H).

### Example 450: Synthesis of 1-cyclopropyl-3-(8-(methylamino)-5-(5-morpholinobenzo[d]oxazol-2-yl)-2,7-naphthyridin-3-yl)urea

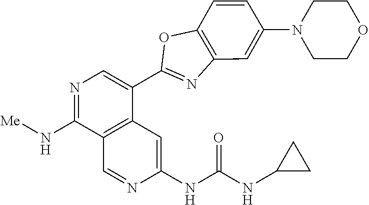 [SMILES: CNc1ncc(-c2nc3cc(N4CCOCC4)ccc3o2)c2cc(NC(=O)NC3CC3)ncc12]

To a stirred solution of N¹-methyl-4-(5-morpholinobenzo[d]oxazol-2-yl)-2,7-naphthyridine-1,6-diamine (Example 421, step 1) (100.0 mg; 0.266 mmol; 1.00 eq.) in DMF (14 mL) was added NaH (60%) (38.2 mg; 1.596 mmol; 6.00 eq.) at 0° C. under nitrogen atmosphere. The resulting mixture was stirred at room temperature for 0.5 hour. To the mixture was added a solution of phenyl cyclopropylcarbamate (61.2 mg; 0.346 mmol, 1.30 eq.) in DMF (1 mL). The mixture was stirred at 80° C. for 4 hours. The desired product was detected via LCMS. The reaction was quenched with saturated. NH₄Cl solution (1 mL), and the solvent was removed under reduced pressure. The residue was purified by flash chromatography on silica gel column using 2-10% of MeOH in CH₂Cl₂ as eluent to afford 1-cyclopropyl-3-(8-(methylamino)-5-(5-morpholinobenzo[d]oxazol-2-yl)-2,7-naphthyridin-3-yl)urea as a yellow solid (28.1 mg, 23.0%). LCMS (ESI) m/z 460.2, [M+H]⁺. ¹H NMR (400 MHz, DMSO-d₆) δ 9.32 (s, 1H), 9.24 (s, 1H), 9.20 (s, 1H), 8.83 (s, 1H), 8.63-8.51 (m, 1H), 7.59 (d, J=8.8 Hz, 1H), 7.44 (s, 1H), 7.24 (d, J=2.0 Hz, 1H), 7.11-7.01 (m, 3.88-3.75 (m, 4H), 3.21-3.11 (m, 4H), 3.07 (d, J=4.4 Hz, 3H), 2.70-2.60 (m, 1H), 0.77-0.67 (m, 2H), 0.54-0.41 (m, 2H).

### Examples 451 and 452: Synthesis of (1S,2R)-2-(fluoromethyl)-N-(8-(methylamino)-5-(5-morpholinobenzo[d]oxazol-2-yl)-2,7-naphthyridin-3-371)cyclopropane-1-carboxamide (Example 451) and (1R,2S)-2-(fluoromethyl)-N-(8-(methylamino)-5-(5-morpholinobenzo[d]oxazol-2-yl)-2,7-naphthyridin-3-yl)cyclopropane-1-carboxamide (Example 452)

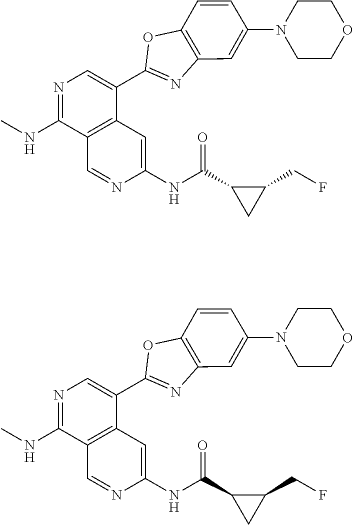 [SMILES: CNc1ncc(-c2nc3cc(N4CCOCC4)ccc3o2)c2cc(NC(=O)[C@H]3C[C@H]3CF)ncc12 ; CNc1ncc(-c2nc3cc(N4CCOCC4)ccc3o2)c2cc(NC(=O)[C@@H]3C[C@@H]3CF)ncc12]

To a stirred solution of N¹-methyl-4-(5-morpholinobenzo[d]oxazol-2-yl)-2,7-naphthyridine-1,6-diamine (Example 421, step 1) (120.0 mg; 0319 mmol; 1.00 eq.) and 2-(fluoromethyl)cyclopropane-1-carboxylic acid (cis racemate) (Example 410, step 3) (33.8 mg; 0.286 mmol; 0.90 eq.) in pyridine (5 mL) was added POCl₃ (146.6 mg; 0.956 mmol; 3.00 eq.) dropwise at 0° C. The resulting mixture was stirred at room temperature for 1 hour. The desired product was detected via LCMS. The solution was quenched with water (2 mL) and extracted with EtOAc (3×20 mL). The combined organic layers were washed with brine (5 mL), dried over anhydrous Na₂SO₄, filtered, and concentrated under reduced pressure. The residue was purified by flash chromatography on silica gel column using 5-10% of MeOH in CH₇Cl₂ as eluent to afford the cis racemate. The cis racemate was separated by Prep-Chiral-SFC (Column: XA-YMC Cellulose-SZ; 4.6×100 mm, 3 um; Mobile Phase A: CO₂; Flow rate: 100 mL/min; Gradient: isocratic 45% B; Column Temperature(° C.): 35; Back Pressure(bar): 100; Wave Length: 220 nm; R_{T1}(min): 6.08; R_{T2}(min): 8.13) to afford (1S,2R)-2-(fluoromethyl)-N-(8-(methylamino)-5-(5-morpholinobenzo[d]oxazol-2-yl)-2,7-naphthyridin-3-yl)cyclopropane-1-carboxamide (Example 451, the faster peak) as a yellow solid (59.4 mg, 37.7%) and (1R,2S)-2-(fluoromethyl)-N-(8-(methylamino)-5-(5-morpholinobenzo[d]oxazol-2-yl)-2,7-naphthyridin-3-yl)cyclopropane-1-carboxamide (Example 452, the slower peak) as a yellow solid (59.4 mg, 37.7%). The two configurations are arbitrarily assigned. LCMS (ESI) m/z 477.2 [M+H]⁺.

HNMR for Example 451: ¹H NMR (400 MHz, DMSO-d₆) δ 10.98 (s, 1H), 9.62 (s, 1H), 944 (s, 1H), 8.89 (s, 1H), 8.87-8.42 (m, 1H), 7.59 (d, J=8.8 Hz, 1H), 7.2.4 (d, J=2.4 Hz, 1H), 7.07 (dd, J=8.8, 2.3 Hz, 1H), 4.90-4.67 (m, 1H), 4.67-4.46 (m, 1H), 3.88-3.62 (m, 4H), 3.21-3.12 (m, 4H), 3.09 (d, J=4.4 Hz, 3H), 2.39-2.27 (m, 1H), 1.90-1.71 (m, 1H), 1.26-1.10 (m, 2H).
HNMR for Example 452: ¹H, NMR (400 MHz, DMSO-d₆) δ 9.62 (s, 1H), 9.44 (s, 1H), 8.89 (s, 1H), 7.58 (d, J=8.8 Hz, 1H), 7.24 (d, J=2.4 Hz, 1H), 7.06 (dd, J=8.8, 2.4 Hz, 1H), 4.88-4.69 (m, 1H), 4.69-4.46 (m, 1H), 3.87-3.71 (m, 4H), 3.20-3.12 (m, 4H), 3.09 (d, J=4.4 Hz, 3H), 2.37-2.30 (m, 1H), 1.85-1.74 (m, 1H), 1.23-1.11 (m, 2H).

### Example 453: Synthesis of N-(5-(5-(3-oxa-8-azabicyclo[3.2.1]octan-8-yl)benzo[d]oxazol-2-yl)-8-((methyl-d₃)amino)-2,7-naphthyridin-3-yl)cyclopropanecarboxamide

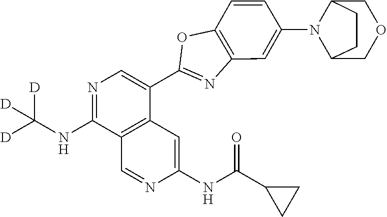 [SMILES: [2H]C([2H])([2H])Nc1ncc(-c2nc3cc(N4C5CCC4COC5)ccc3o2)c2cc(NC(=O)C3CC3)ncc12]

### Step 1: 5-(3-oxa-8-azabicyclo[3.2.1]octan-8-yl)-2-methylbenzo[d]oxazole

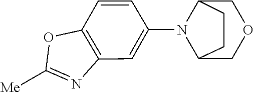 [SMILES: Cc1nc2cc(N3C4CCC3COC4)ccc2o1]

To a stirred solution of 3-oxa-8-azabicyclo[3.2.1]octane hydrochloride (1.56 g; 11.176 mmol; 1.60 eq.) and 5-bronco-2-methylbenzo[d]oxazole (1.50 g; 6,958 mmol; 1.00 eq.) in 1,4-dioxane (10 mL) were added. BrettPhos (687.2 mg; 1.227 mmol; 0.20 eq.), BrettPhos Pd. G₃ (579.0 mg; 0.613 mmol; 0.10 eq.) and Cs₂CO₃ (6.90 g; 21.059 mmol; 3.00 eq.). The mixture was stirred at 110° C. overnight under nitrogen atmosphere. The desired product was detected via LCMS. The mixture was allowed to cool down to room temperature. The resulting mixture was concentrated under reduced pressure. The residue was purified by flash chromatography on silica gel column using 10-30% of EtOAc in petroleum ether as eluent to provide 5-(3-oxa-8-azabicyclo[3.2.1]octan-8-yl)-2-methylbenzo[d]oxazole as a yellow solid (180.0 mg, 13.8%). LCMS (ESI) m/z 245.1; [M+H]⁺.

### Step 2: 2-amino-4-(3-oxa-8-azabicyclo[3.2.1]octan-8-yl)phenol

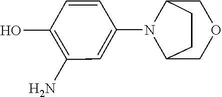 [SMILES: Nc1cc(N2C3CCC2COC3)ccc1O]

To a solution of 5-(3-oxa-8-azabicyclo[3.2.1]octan-8-yl)-2-methylbenzo[d]oxazole (180.0 mg; 0.737 mmol; 1.00 eq.) in EtOH (5 mL) was added a solution of HCl (4 M in water, 2 mL). The mixture was stirred at 100° C. for 4 hours under nitrogen atmosphere. The desired product was detected via LCMS. The mixture was allowed to cool down to room temperature and concentrated under reduced pressure to afford 2-amino-4-(3-oxa-8-azabicyclo[3.2.1]octan-8-yl)phenol as a brown solid (160.2 mg, crude). The crude product was taken directly to the next reaction without further purification. LCMS (ESI) m/z 221.1, [M+H]⁺.

Step 3: 5-(3-oxa-8-azabitcyclo[3.2.1]octan-8-yl)benzo[d]oxazole

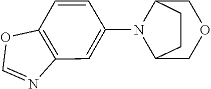 [SMILES: c1nc2cc(N3C4CCC3COC4)ccc2o1]

A solution of 2-amino-4-(3-oxa-8-azabicyclo[3.2.1]octan-8-yl)phenol. (160.2 mg; 0.817 mmol; 1.00 eq.) in trimethoxymethane (5 mL) was stirred at 110° C. overnight under nitrogen atmosphere. The desired product was detected via LCMS. The mixture was allowed to cool down to room to temperature and concentrated under reduced pressure. The residue was purified by flash chromatography on silica gel column using 20-30% of EtOAc in petroleum ether as eluent to provide 5-(3-oxa-8-azabicyclo[3.2.1]octan-8-yl)benzo[d]oxazole as a brown solid (120.0 mg, 65.7%). LCMS (ESI) m/z 231.1, [M+H]⁺.

### Step 4: 11-(56(5-(3-oxa-8-azabicyclo[3.2.1]-octan-8-yl)benzo[d]oxazol-2-yl)-8-((methyl-d₃)amino)-2,7-naphthyridin-3-yl)cyclopropanecarboxamide

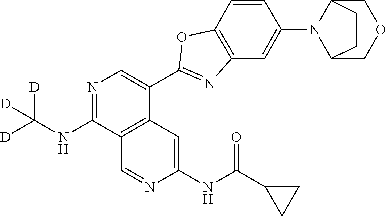 [SMILES: [2H]C([2H])([2H])Nc1ncc(-c2nc3cc(N4C5CCC4COC5)ccc3o2)c2cc(NC(=O)C3CC3)ncc12]

To a stirred solution of 5-(3-oxa-8-azabicyclo[3.2.1]octan-8-yl)benzo[d]oxazole (49.0 mg; 0.213 mmol 1.20 eq.) and N-(5-bromo-8-((methyl-d₃)amino)-2,7-naphthyridin-3-yl)cyclopropanecarboxamide (60.2 mg; 0.185 mmol; 1.00 eq.) in DMF (1 mL) were added Pd(PPh₃)₄ (21.3 mg; 0.018 mmol; 0.10 eq.) and Cs₂CO₃ (180.3 mg; 0.553 mmol; 3.00 eq.). The resulting mixture was stirred at 110° C. overnight under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide the crude product. The crude product was recrystallized with a mixture of solvent (CH₂Cl₂/MeOH, 1:1, 5 mL) to afford N-(5-(5-(3-oxa-8-azabicyclo[3.2.1]octan-8-yl)benzo[d]oxazol-2-yl)-8-((methyl-d₃)amino)-2,7-naphthyridin-3-yl)cyclopropanecarboxamide as a yellow solid (75.4 mg, 86.0%). LCMS (ESI) m/z 474.2, [M+H]⁺. ¹H NMR (400 MHz, DMSO-d₆) 11.02. (s, 1H), 9.59 (s, 1H), 9.43 (s, 1H), 8.86 (s, 1H), 8.59 (5, 1H), 7.53 (d, J=8.8 HZ, 1H), 7.16 (d, J=2.4 HZ, 1H), 6.94 (dd, J=8.8, 2.4 Hz, 1H), 4.26-4.20 (m, 2H), 3.79 (d, J=10.4 Hz, 2H), 3.48 (d, J=10.4 Hz, 2H), 2.15-2.06 (m, 1H), 1.99-1.89 (m, 4H), 0.95-0.81 (m, 4H).

### Example 454: Synthesis of N-(5-(5-(cyclopropylmethoxy)benzo[d]oxazol-2-yl-8-((methyl-d₃)amino)-2,7-naphthyridin-3-yl)cyclopropanecarboxamide

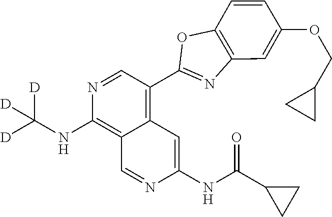 [SMILES: [2H]C([2H])([2H])Nc1ncc(-c2nc3cc(OCC4CC4)ccc3o2)c2cc(NC(=O)C3CC3)ncc12]

### Step 1: 5-(cyclopropylmethoxy)benzo[d]oxazole

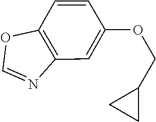 [SMILES: c1nc2cc(OCC3CC3)ccc2o1]

To a solution of benzo[d]oxazol-5-ol (200.0 mg; 1.480 mmol; 1.00 eq.) and Cs₂CO₃ (10.45 g; 4.440 mmol, 3.00 eq.) in DMF (5.0 mL) was added (bromomethyl)cyclopropane (299.7 mg; 2.220 mmol; 1.50 eq.). The reaction was stirred at room temperature for 2 hours. The desired product was detected via LCMS. The resulting mixture was diluted with EtOAc (60 mL), washed with brine (3×5 mL), and concentrated under vacuum. The residue was purified by flash chromatography on pre-packed C18 column using 70-90% of MeOH in water (10 mmol/L NH₄HCO₃) as eluent to provide 5-(cyclopropylmethoxy)benzo[d]oxazole as a yellow oil (267.6 mg, 95.2%). LCMS (ESI) m/z 190.1, [M+H]⁺.

### Step 2: N-(5-(5-(cyclopropylmethoxy)benzo[d]oxazol-2-yl)-8-((methyl-d₃)amino)-2,7-naphthyridin-3-yl)cyclopropanecarboxamide

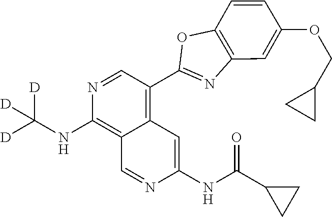 [SMILES: [2H]C([2H])([2H])Nc1ncc(-c2nc3cc(OCC4CC4)ccc3o2)c2cc(NC(=O)C3CC3)ncc12]

A solution of 5-(cyclopropylinethoxy)benzo[d]oxazole (70.0 mg; 0.370 mmol; 1.50 eq.), N-(5-bromo-8-((methyl-613)amino)-2,7-naphthyridin-3-yl)cyclopropanecarboxamide (80.0 mg; 0.247 mmol; 1.00 eq.), Pd(PPh₃)₄ (17.1 mg; 0.015 mmol; 0.06 eq.) and Cs₂CO₃ (241.2 mg; 0.740 mmol; 3.00 eq.) in DMF (5 mL) was stirred at 110° C. for 2 hours under nitrogen atmosphere. The desired product was detected via LCMS. The resulting mixture was concentrated under reduced pressure. The residue was purified by flash chromatography on silica gel column using 0-5% of MeOH iii CH₂Cl₂ as eluent to provide N-(5-(5-(cyclopropylmethoxy)benzo[d]oxazol-2-yl)-8-((methyl-d₃)amino)-2,7-naphthyridin-3-yl)cyclopropanecarboxamide as a light yellow solid (63.7 mg, 59.6%). LCMS (ESI) m/z 433.2, [M+H]⁺. ¹H NMR (400 MHz, DMSO-d₆) δ 11.04 (s, 1H), 9.64 (s, 1H), 9.43 (s, 1H), 8.89 (s, 1H), 8.63 (s, 1H), 7.61 (d, J=8.8 Hz, 1H), 7.24 (d, J=2.4 Hz, 1H), 6.96 (dd, J=8.8, 2.4 Hz, 1H), 3.91 (d, J=6.8 Hz, 2H), 2.15-2.05 (m, 1H), 1.30-1.22 (m, 1H), 0.93-0.83 (m, 4H), 0.62-0.57 (m, 2H), 0.40-0.32 (m, 2H).

### Example 455: Synthesis of (1S,2R)—N-(5-(5-(3-oxa-8-azabicyclo[3.2.1]octan-8-yl)benzo[d]oxazol-2-yl)-8-((methyl-d₃)amino)-2,7-naphthyridin-3-yl)-2-methylcyclopropane-1-carboxamide

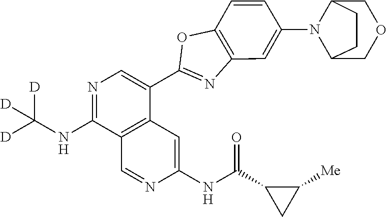 [SMILES: [2H]C([2H])([2H])Nc1ncc(-c2nc3cc(N4C5CCC4COC5)ccc3o2)c2cc(NC(=O)[C@H]3C[C@H]3C)ncc12]

### Step 1: (1S,2R)-11-(5-bromo-8-((methyl-d₃)amino)-2,7-naphthyridin-3-yl)-2-methylcyclopropane-1-carboxamide

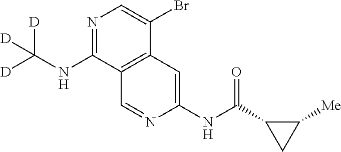 [SMILES: [2H]C([2H])([2H])Nc1ncc(Br)c2cc(NC(=O)[C@H]3C[C@H]3C)ncc12]

To a solution of (1S,2R)-2-methyl-N-(8-((methyl-d₃)amino)-2,7-naphthyridin-3-yl)cyclopropane-1-carboxamide (500.1 mg; 1.928 mmol, 1.00 eq.) in DMF (5 mL) was added NBS (343.2 mg; 1,928 mmol; 1.00 eq.) in portions at 0° C. The resulting mixture was stirred at room temperature for 1 hour under nitrogen atmosphere. The desired product was detected via LCMS. The mixture was added dropwise into water (30 mL), whereupon the precipitated product was isolated via filtration, washed with water (3×10 mL) and dried under reduced pressure to afford (1S,2R)—N-(5-bromo-8-((methyl-d₃)amino)-2,7-naphthyridin-3-yl)-2-methylcyclopropane-1-carboxamide as a yellow solid (480.1 mg, 73.5%). LCMS (ESI) m/z 338.1 [M+H]⁺.

### Step 2: (1S,2R)—N-05-(5-(3-oxa-8-azabicyclo[3.2.1]octan-8-yl)benzo[d]oxazol-2-yl)-8-((methyl 43)amino)-2,7-naphthyridin-3-yl)-2-methylcyclopropane-1-carboxamide

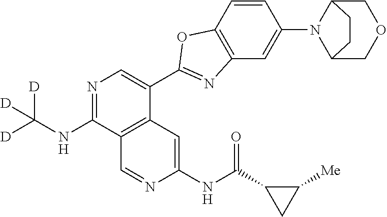 [SMILES: [2H]C([2H])([2H])Nc1ncc(-c2nc3cc(N4C5CCC4COC5)ccc3o2)c2cc(NC(=O)[C@H]3C[C@H]3C)ncc12]

To a stirred solution of (1S,2R)—N-(5-bromo-8-((methyl-d₃)amino)-2,7-naphthyridin-3-yl)-2-methylcyclopropane-1-carboxamide (60.2 mg; 0.177 mmol, 1.00 eq.) and 5-(3-oxa-8-azabicyclo[3.2.1]octan-8-yl)benzo[d]oxazole (Example 453, step 3) (48.6 mg; 0.211 mmol; 1.19 eq.) in DMF (1 mL) were added Pd(PPh₃)₄. (20.5 mg; 0.018 mmol; 0.10 eq.) and Cs₂CO₃ (173.9 mg; 0.534 mmol; 3.00 eq.). The resulting mixture was stirred at 110° C. overnight under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide the crude product. Then the crude product was recrystallized with a mixture solvent of CH₂Cl₂/MeOH (1:1, 5 mL) to afford (1S,2R)—N-(5-(5-(3-oxa-8-azabicyclo[3.2.1]octan-8-yl)benzo[d]oxazol-2-yl)-8-((methyl-d₃)amino)-2,7-naphthyridin-3-yl)-2-methylcyclopropane-1-carboxamide as a yellow solid (62.3 mg, 72.0%). LCMS (ESI) m/z 488.2, [M+H]⁺. ¹H NMR (400 MHz, DMSO-d₆) 10.90 (s, 1H), 9.55 (s, 1H), 9.42 (s, 1H), 8.86 (s, 1H), 8.58 (s, 1H), 7.53 (d, J=8.8 Hz, 1H), 7.17 (d, J=2.4 Hz, 1H), 6.95 (dd, J=8.8, 2.4 Hz, 1H), 4.26-4.20 (m, 2H), 3.79 (d, J=10.4 Hz, 2H), 3.48 (d, J=10.4 Hz, 2H), 2.15-2.07 (m, 1H), 1.99-1.89 (m, 4H), 1.35-1.27 (m, 1H), 1.16 (d, J=6.0 Hz, 3H), 1.05-0.98 (m, 1H), 0.91-0.85 (m, 1H).

### Example 456: Synthesis of (1S,2R)—N-(5-(5-(cyclopropylmethoxy)benzo[d]oxazol-2-yl)-8-((methyl-d₃)amino)-2,7-naphthyridin-3-yl)-2-methylcyclopropane-1-carboxamide

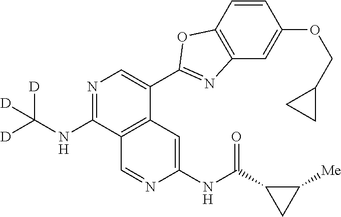 [SMILES: [2H]C([2H])([2H])Nc1ncc(-c2nc3cc(OCC4CC4)ccc3o2)c2cc(NC(=O)[C@H]3C[C@H]3C)ncc12]

A solution of 5-(cyclopropylmethoxy)benzo[d]oxazole (Example 454, step 1) (53.7 mg; 0.284 mmol; 1.20 eq.), (1S,2R)—N-(5-bromo-8-((methyl-d₃)amino)-2,7-naphthyridin-3-yl)-2-methylcyclopropane-1-carboxamide (Example 455, step 1) (80.0 mg; 0.237 mmol; 1.00 eq.), Pd(PPh₃)₄ (16.4 mg; 0.014 mmol; 0.06 eq.) and Cs₂CO₃ (231.2 mg; 0.711 mmol; 3.00 eq.) in DMF (4.0 mL) was stirred at 110° C. for 2 hours under nitrogen atmosphere. The desired product was detected via LCMS. The resulting mixture was concentrated under vacuum. The residue was purified by flash chromatography on silica gel column using 0-5% of MeOH in CH₂Cl₂ as eluent to provide (1S,2R)—N-(5-(5-(cyclopropylmethoxy)benzo[d]oxazol-2-yl)-8-((methyl-d₃)amino)-2,7-naphthyridin-3-yl)-2-methylcyclopropane-1-carboxamide as a yellow solid (63.5 mg, 60.0%). LCMS (ESI) m/z 447.2, [M+H]⁺. ¹H NMR (400 MHz, DMSO-d₆) δ 10.91 (s, 1H), 9.61 (s, 1H), 9.42 (s, 1H), 8.88 (s, 1H), 8.60 (s, 1H), 7.61 (d, 8.8 Hz, 1H), 7.24 (d, J=2.4 Hz, 1H), 6.97 (dd, J=8.8, 2.4 Hz, 1H), 3.91 (d, J=6.8 Hz, 2H), 2.16-2.08 (m, 1H), 1.36-1.24 (m, 2H), 1.16 (d, J=6.0 Hz, 3H), 1.04-0.99 (m, 1H), 0.92-0.89 (m, 1H), 0.62-0.57 (m, 2H), 0.40-0.34 (m, 2H).

### Example 457: Synthesis of N-(8-(ethylamino)-5-(5-(7-methyl-9-oxa-3,7-diazabicyclo[3.11]nonan-3-yl)benzo[d]oxazol-2-yl)-2,7-naphthyridin-3-yl)cyclopropanecarboxamide

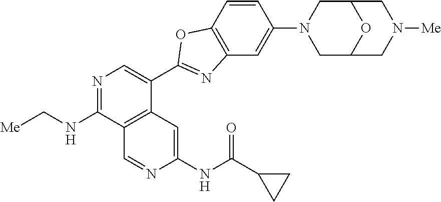 [SMILES: CCNc1ncc(-c2nc3cc(N4CC5CN(C)CC(C4)O5)ccc3o2)c2cc(NC(=O)C3CC3)ncc12]

### Step 1: tert-bated 7-(2-methylbenzo[d]oxazol-5-yl)-9-oxa-3,7-diazabicyclo[3.3.1]nonane-3-carboxylate

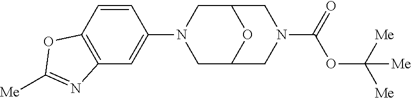 [SMILES: Cc1nc2cc(N3CC4CN(C(=O)OC(C)(C)C)CC(C3)O4)ccc2o1]

A stirred solution of 5-bromo-2-methylbenzo[d]oxazole (100.0 mg; 0.472 mmol; 1.0) eq.), tert-butyl 9-oxa-3,7-diazabicyclo[3.3.1]nonane-3-carboxylate (172.3 mg; 0.755 mmol; 1.60 eq.), BrettPhos (45.6 mg; 0.085 mmol; 0.18 eq.), BrettPhos Pd G₃ (38.5 mg; 0.042 mmol; 0.09 eq.), Cs₂CO₃ (399.5 mg; 1.226 mmol; 2.60 eq.) in 1,4-dioxane (4 mL) was stirred at 100° C. overnight under nitrogen atmosphere. The desired product was detected via LCMS. The solvent was concentrated under vacuum. The residue was purified by flash chromatography on silica gel column using 2-10% of EtOAc in petroleum ether as eluent to provide tert-butyl 7-(2-methylbenzo[d]oxazol-5-yl)-9-oxa-3,7-diazabicyclo[3.3.1]nonane-3-carboxy late as a white solid (95.0 mg, 56.1%). LCMS (ESI) m/z 360.2, [M+H]⁺.

### Step 2: 3-(2-methylbenzo[d]oxazol-5-yl)-9-oxa-3,7-diazabicyclo[3.3.1]nonane 2,2,2-trifluoroacetate

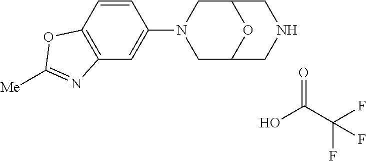

To a stirred solution of tort-butyl 7-(2-methylbenzo[d]oxazol-5-yl)-9-oxa-3,7-diazabicyclo[3.3.1]nonane-3-carboxylate (95.0 mg; 0.264 mmol; 1.00 eq.) in CH₂Cl₂ (5 mL) was added 2,2,2-trifluoroacetic acid (1 mL). The resulting mixture was stirred at room temperature for 0.5 hour. The desired product was detected via LCMS. The solvent was concentrated under reduced pressure to provide 3-(2-methylbenzo[d]oxazol-5-yl)-9-oxa-3,7-diazabicyclo[3.3.1]nonane 2,2,2-trifluoroacetate as a yellow solid (68.0 mg, crude). LCMS (ESI) m/z 260.1, [M+H]⁺.

### Step 3: 3-methyl-7-(2-methylbenzo[d]oxazol-5-371)-9-oxa-3,7-diazabicyclo[3.3.1]nonane

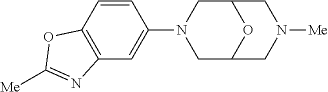 [SMILES: Cc1nc2cc(N3CC4CN(C)CC(C3)O4)ccc2o1]

To a stirred solution of 3-(2-methylbenzo[d]oxazol-5-yl)-9-oxa-3,7-diazabicyclo[3.3.1]nonane 2,2,2-trifluoroacetate (68.0 mg 0.262 mmol; 1.00 eq.) and DIEA (33.9 mg; 0.262 mmol; 1.00 eq.) in CH₂Cl₂ (5 mL) was added HCHO (35% w/w in water, 22.4.5 mg). The resulting solution was stirred at room temperature for 0.5 hour. To the mixture was added NaBH(OAc)₃ (166.7 mg; 0.787 mmol; 3.00 eq.). The mixture was stirred at room temperature for 2 hours. The reaction was quenched by water (2 mL) and the mixture was concentrated under reduced pressure. The residue was purified by flash chromatography on silica gel column using 2-10% of MeOH in CH₂Cl₂ (0.1% Et₃N) to afford 3-methyl-7-(2-methylbenzo[d]oxazol-5-yl)-9-oxa-3,7-diazabicyclo[3.3.1]nonane as a white solid (30.0 mg, 20.9%), LCMS (ESI) m/z 274.2, [M+H]+.

### Step 4: 2-amino-4-(7-methyl-9-oxa-3,7-diazabicyclo[3.3.1]nonan-3-yl)phenol

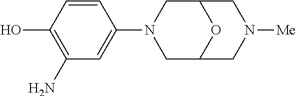 [SMILES: CN1CC2CN(c3ccc(O)c(N)c3)CC(C1)O2]

To a stirred solution of 3-methyl-7-(2-methylbenzo[d]oxazol-5-yl)-9-oxa-3,7-diazabicyclo[3.3.1]nonane (30.0 mg; 0.110 mmol; 1.00 eq.) m EtOH (5 mL) was added HCl (4 M in water, 2 mL). The mixture was stirred at 100° C. for 4 hours under nitrogen atmosphere. The desired product was detected via. LCMS. The solvent was concentrated under reduced pressure to provide 2-amino-4-(7-methyl-9-oxa-3,7-diazabicyclo[3.3.1]nonan-3-yl)phenol as a yellow solid. (30.0 mg, crude). LCMS (ESI) m/z 250.2; [M+H]⁺.

### Step 5: 3-(benzo[d]oxazol-5-yl)-7-methyl-9-oxa-3,7-diazabicyclo[3.3.1]nonane

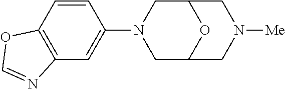 [SMILES: CN1CC2CN(c3ccc4ocnc4c3)CC(C1)O2]

A stirred solution of 2-amino-4-(7-methyl-9-oxa-3,7-diazabicyclo[3.3.1]nonan-3-yl)phenol (40.0 mg; 0.160 mmol; 1.00 eq.) in trimethyl orthoformate (3 mL) was stirred at 100° C. overnight under nitrogen atmosphere. The resulting mixture was cooled and concentrated under reduced pressure. The residue was purified by flash chromatography on silica gel column using 2-10% of MeOH in CH₂Cl₂ to afford 3-(benzo[d]oxazol-5-yl)-7-methyl-9-oxa-3,7-diazabicyclo[3.3.1]nonane as a yellow solid (37.0 mg, 88.9%), LCMS (ESI) m/z 260.2, [M+H]⁺.

### Step 6: N-(8-(ethylamino)-5-(5-(7-methyl-9-oxa-3,7-diazabicyclo[3.3.1]nonan-3-yl)benzo[d]oxazol-2-yl)-2,7-naphthyridin-3-yl)cyclopropanecarboxamide

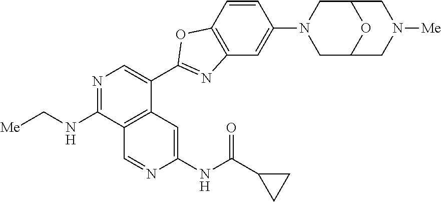 [SMILES: CCNc1ncc(-c2nc3cc(N4CC5CN(C)CC(C4)O5)ccc3o2)c2cc(NC(=O)C3CC3)ncc12]

A stirring mixture of 3-(benzo[d]oxazol-5-yl)-7-methyl-9-oxa-3,7-diazabicyclo[3.3.1]nonane (32.5 mg; 0.125 mmol, 1.20 eq.), N-(5-bromo-8-(ethylamino)-2,7-naphthyridin-3-yl)cyclopropanecarboxamide (35.0 mg; 0.104 mmol; 1.00 eq.), Pd(PPh₃)₄ (12.1 mg; 0.010 mmol; 0.10 eq.), Cs₂CO₃ (102.1 mg; 0.312 mmol; 3.00 eq.) in DMF (2 mL) was stirred at 110° C. for 14 hours under nitrogen atmosphere. The desired product was detected via LCMS. The solvent was concentrated under vacuum. The residue was purified by flash chromatography on silica gel column using 2-10% of MeOH in CH₂Cl₂ to afford a crude product. The crude product was purified by flash chromatography on pre-packed C18 column using 20-60% of MeCN in water (10 mmol/L NH₄HCO₃) to provide N-(8-(ethylamino)-5-(5-(7-methyl-9-oxa-3,7-diazabicyclo[3.3.1]nonan-3-yl)benzo[d]oxazol-2-yl)-2,7-naphthyridin-3-yl)cyclopropanecarboxamide as a yellow solid (9.1 mg, 16.5%). LCMS (ESI) m/z 514.2, [M+H]⁺. ¹H NMR (400 MHz, DMSO-d₆) δ 11.04 (s, 1H), 9.68 (s, 1H), 9.48 (s, 1H), 8.88 (s, 1H), 8.66-8.51 (m, 1H), 7.59 (d, J=8.4 Hz, 1H), 7.20-6.80 (m, 2H), 4.12-4.05 (m, 2H); 3.70-3.62 (m, 4H), 3.11-3.06 (m, 2H), 2.90-2.88 (m, 2H), 2.38-2.30 (m, 2H), 2.20-2.00 (m, 4H), 1.28 (t, J=6.8 Hz, 3H), 0.94-0.82 (m, 4H).

### Example 458: Synthesis of N-(8-(ethylamino)-5-(5-morpholinobenzo[d]oxazol-2-yl)-2,7-naphthyridin-3-yl)cyclopropanecarboxamide

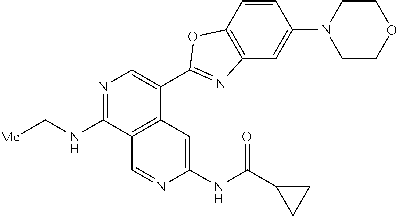 [SMILES: CCNc1ncc(-c2nc3cc(N4CCOCC4)ccc3o2)c2cc(NC(=O)C3CC3)ncc12]

A stirred solution of N-(5-bromo-8-(ethylamino)-2,7-naphthyridin-3-yl)cyclopropanecarboxamide (300.0 mg; 0.895 mmol; 1.00 eq.), Pd(PPh₃)₄ (103.7 mg; 0.090 mmol; 0.10 eq.), Cs₂CO₃ (878.4 mg; 2.696 mmol; 3.01 eq.) and 5-morpholinobenzo[d]oxazole (Example 345, step 3) (220.0 mg; 1.077 mmol; 1.20 eq.) in DMF (5 mL) was stirred at 110° C. for 16 hours under nitrogen atmosphere. The reaction mixture was cooled to room temperature and concentrated under vacuum. The residue was purified by flash chromatography on silica gel column using 2-10% of MeOH in CH₂Cl₂ as eluent to provide the crude product. The product was purified by flash chromatography on pre-packed C18 column using 20-100% of MeOH in water (10 mmol/L NH₄HCO₃) as eluent to provide N-(8-(ethylamino)-5-(5-morpholinobenzo[d]oxazol-2-yl)-2,7-naphthyridin-3-yl)cyclopropanecarboxamide as a yellow solid (309.7 mg, 75.4%). LCMS (ESI) m/z 459.2, [M+H]⁺. ¹H NMR (400 MHz, DMSO-d₆) δ 11.05 (s, 1H), 9.65 (s, 1H), 9.48 (s, 1H), 8.86 (s, 1H), 8.60-8.53 (m, 1H), 7.58 (d, J=8.8 Hz, 1H), 7.23 (d, J=2.4 Hz, 1H), 7.11-7.02 (m, 1H), 3.84-3.75 (m, 4H), 3.70-3.60 (m, 2H), 3.21-3.12 (m, 4H), 2.15-2.07 (m, 1H), 1.28 (t, J=7.2 Hz, 3H), 0.94-0.83 (m, 4H).

### Example 459: Synthesis of N-(8-(ethylamino)-5-(5-methoxybenzo[d]oxazol-2-yl)-2,7-naphthyridin-3-yl)cyclopropanecarboxamide

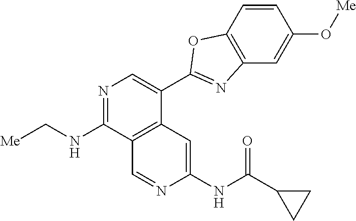 [SMILES: CCNc1ncc(-c2nc3cc(OC)ccc3o2)c2cc(NC(=O)C3CC3)ncc12]

A stirred solution of 5-methoxybenzo[d]oxazole (107.0 mg; 0.717 mmol; 1.20 eq.), N-(5-bromo-8-(ethylamino)-2,7-naphthyridin-3-yl)cyclopropanecarboxamide (200.0 mg; 0.597 mmol; 1.00 eq.), Pd(PPh₃)₄ (69.2 mg; 0.060 mmol; 0.10 eq.) and Cs₂CO₃ (585.6 mg; 1.797 mmol; 3.01 eq.) in DMF (5 mL) was stirred at 110° C. for 16 hours under nitrogen atmosphere. The desired product was detected via LCMS. The resulting mixture was concentrated under vacuum. The residue was purified by flash chromatography on silica gel column using 2-10% of MeOH in CH₂Cl₂ as eluent to provide the crude product. The crude product was purified by flash chromatography on pre-packed C18 column using 20-100% of MeOH in water (10 mmol/NH₄HCO₃) as eluent to provide N-(8-(ethylamino)-5-(5-methoxybenzo[d]oxazol-2-yl)-2,7-naphthyridin-3-yl)cyclopropanecarboxamide as a yellow solid (85.0 mg, 35.3%), LCMS (ESI) m/z 404.2, [M+H]⁺. ¹H NMR (400 MHz, DMSO-d₆) 11.06 (s, 1H), 9.65 (s, 1H), 9.48 (s, 1H), 8.88 (s, 1H), 8.63 8.56 (m, 1H), 7.62 (d, J=8.8 Hz, 1H), 7.28 (d, J=2.4 Hz, 1H), 7.01-6.92 (m, 1H), 3.86 (s, 3H), 3.69-3.60 (m, 2H), 2.18-2.07 (m, 1.28 (t, J=7.2 Hz, 3H), 0.94-0.82 (m, 4H).

### Example 460: Synthesis of 1V-(8-(methylamino)-5-(5-methyloxazolo[4,5-b]pyridin-2-yl)-2,7-naphthyridin-3-yl)cyclopropanecarboxamide

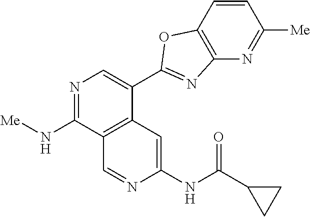 [SMILES: CNc1ncc(-c2nc3nc(C)ccc3o2)c2cc(NC(=O)C3CC3)ncc12]

### Step 1: 5-methyloxazolo[4,5-b]pyridine

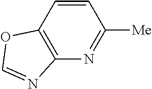

A solution of 2-amino-6-methylpyridin-3-ol (300.0 mg; 2.417 mmol; 1.00 eq.) in trimethyl orthoformate (8 mL) was stirred at 100° C. for 14 hours. The desired product was detected via LCMS. The resulting mixture was concentrated under reduced pressure. The residue was purified by flash chromatography on silica gel column using 30-70% of EtOAc in petroleum ether as eluent to provide 5-methyloxazolo[4,5-b]pyridine as a yellow solid (227.6 mg, 70.2%). LCMS (ESI) m/z 135.0, [M+H]⁺.

### Step 2: N-(8-(methylamino)-5-(5-methyloxazolo[4,5-b]pyridin-2-yl)-2,7-naphthyridin-3-yl)cyclopropanecarboxamide

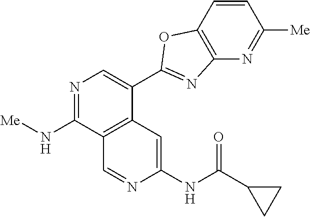 [SMILES: CNc1ncc(-c2nc3nc(C)ccc3o2)c2cc(NC(=O)C3CC3)ncc12]

A solution of N-(5-bromo-8-(methylamino)-2,7-naphthyridin-3-yl)cyclopropanecarboxamide (80.0 mg; 0.249 mmol; 1.00 eq.), 5-methyloxazolo[4,5-b]pyridine (50.4 mg; 0.376 mmol; 1.51 eq.), Pd(PPh₃)₄ (17.3 mg; 0.015 mmol; 0.06 eq.) and Cs₂CO₃ (243.5 mg; 0.747 mmol; 3.00 eq.) in DMF (5 mL) was stirred at 110° C. for 2 hours under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide N-(8-(methylamino)-5-(5-methyloxazolo[4,5-b]pyridin-2-yl)-2,7-naphthyridin-3-yl)cyclopropanecarboxamide as an off-white solid (30.0 trig, 32.1%). LCMS (ESI) m/z 375.1, [M+H]⁺. ¹H NMR (400 MHz, DMSO-d₆) δ 11.05 (s, 1H), 9.75 (s, 1H), 9.45 (s, 1H), 8.97 (s, 1H), 8.79-8.74 (m, 1H), 8.03 (d, J=8.0 Hz, 1H), 7.26 (d, J=8.0 Hz, 1H), 3.11 (d, J=4.4 Hz, 3H), 2.60 (s, 3H), 2.15-2.08 (m, 1H), 0.97-0.91 (m, 2H), 0.90-0.83 (m, 2H).

### Example 461: Synthesis of N-(5-(6-(difluoromethyl)oxazolo[4,5-d]pyridin-2-yl)-8-(methylamino)-2,7-naphthyridin-3-yl)cyclopropanecarboxamide

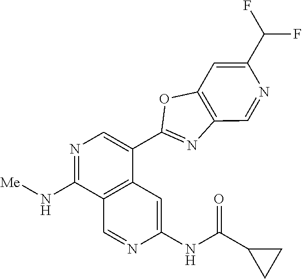 [SMILES: CNc1ncc(-c2nc3cnc(C(F)F)cc3o2)c2cc(NC(=O)C3CC3)ncc12]

### Step 1: 5-amino-1-(difluoromethyl)pyridin-4-ol

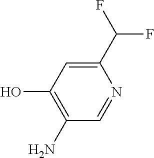 [SMILES: Nc1cnc(C(F)F)cc1O]

To a solution of 2-(difluoromethyl)-5-nitropyridin-4-ol (200.0 mg, 1.052 mmol; 1.00 eq) in EtOH (20 mL) was added 20% Pd(OH)₂/C (40.0 mg; 20% w/w) under nitrogen atmosphere. The resulting mixture was stirred at room temperature for 3 hours under hydrogen atmosphere (2 atm). The desired product was detected via LCMS. The resulting mixture was filtered, and concentrated under reduced pressure to afford 5-amino-2-(difluoromethyl)pyridin-4-ol as a yellow solid (160 mg, crude). LCMS (ESI) m/z 161.0, [M+H]⁺.

### Step 2: 6-(difluoromethyl)oxazolo[4,5-c]pyridine

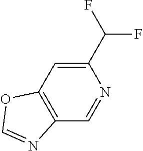 [SMILES: FC(F)c1cc2ocnc2cn1]

A solution of 5-amino-2-(difluoromethyl)pyridin-4-ol (160.0 mg; 0.999 mmol; 1.00 eq) in trimethyl orthoformate (5 mL) was stirred at 100° C. overnight under nitrogen atmosphere. The desired product was detected via LCMS. The resulting mixture was concentrated under reduced pressure. The residue was purified by flash chromatography on silica gel column using 0-15% of MeOH in CH₂Cl₂ as eluent to afford 6-(difluoromethyl)oxazolo[4,5-c]pyridine as an off-white solid (90 mg, 50.3%). LCMS (ESI) m/z 171.0, [M+H]⁺.

### Step 3: N-(5-(6-(difluoromethyl)oxazolo[4,5-c]pyridin-2-yl)-8-(methylamino)-2,7-naphthyridin-3-yl)cyclopropanecarboxamide

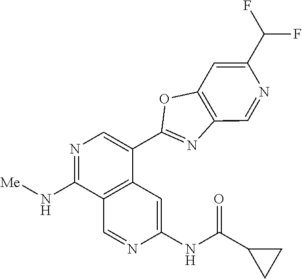 [SMILES: CNc1ncc(-c2nc3cnc(C(F)F)cc3o2)c2cc(NC(=O)C3CC3)ncc12]

N-(5-(6-(difluoromethyl)oxazolo[4,5-c]pyridin-2-yl)-8-(methy amino)-2,7-naphthyridin-3-yl)cyclopropanecarboxamide was synthesized using a similar procedure that was previously described in example 345, by using N-(5-bromo-8-(methylamino)-2,7-naphthyridin-3-yl)cyclopropanecarboxamide and 6-(difluoromethyl)oxazolo[4,5-c]pyridine as the starting material. LCMS (ESI) m/z 411.1, [M+H]⁺. ¹H NMR (400 MHz, DMSO-d₆) δ 11.10 (s, 1H), 9.71 (s, 1H), 9.46 (s, 1H), 9.11 (s, 1H), 9.00 (s, 1H), 8.58-8.46 (m, 1H), 8.15 (s, 1H), 7.11 (t, J=55.2 Hz, 1H), 3.11 (d, J=4.4 Hz, 3H), 2.13-2.09 (m, 1H), 0.94-0.88 (m, 4H).

### Examples 462 and 463: Synthesis of (Z)—N-(5-(5-(1-(methoxyimino)ethyl)benzo[d]oxazol-2-yl)-8-(methylamino)-2,7-naphthyridin-3-yl)cyclopropanecarboxamide (Example 462) and (E)-N-(5-(5-(1-(methoxyimino)ethyl)benzo[d]oxazol-2-yl)-8-(methylamino)-2,7-naphthyridin-3-yl)cyclopropanecarboxamide (Example 463)

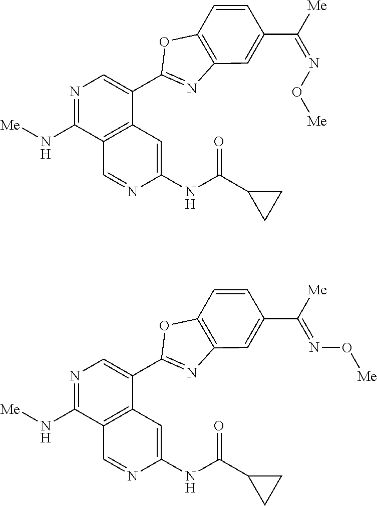 [SMILES: CNc1ncc(-c2nc3cc(/C(C)=N\OC)ccc3o2)c2cc(NC(=O)C3CC3)ncc12 ; CNc1ncc(-c2nc3cc(/C(C)=N/OC)ccc3o2)c2cc(NC(=O)C3CC3)ncc12]

### Step 1: N-(5-(5-acetylbenzo[d]oxazol-2-yl)-8-(methylamino)-2,7-naphthyridin-3-yl)cyclopropanecarboxamide

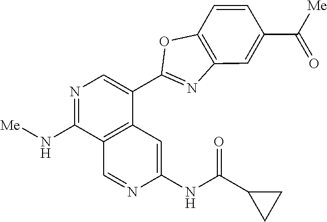 [SMILES: CNc1ncc(-c2nc3cc(C(C)=O)ccc3o2)c2cc(NC(=O)C3CC3)ncc12]

A solution of N-(5-bromo-8-(methylamino)-2,7-naphthyridin-3-yl)cyclopropanecarboxamide (2000) mg; 0.623 mmol; 1.00 eq.), 1-(benzo[d]oxazol-5-yl)ethan-1-one (120.0 mg; 0.745 mmol; 1.20 eq.), Pd(PPh₃)₄ (72.2 mg; 0.062 mmol; 0.10 eq.) and Cs₂CO₃ (611.2 mg; 1.876 mmol; 3.01 eq.) in DMF (5 mL) was stirred at 110° C. for 2 hours under nitrogen atmosphere. LC-MS indicated that the starting material was consumed. The resulting mixture was concentrated under vacuum. The residue was purified by flash chromatography on silica gel column using 2-10% of MeOH in CH₂Cl₂ as eluent to provide N-(5-(5-acetylbenzo[d]oxazol-2-yl)-8-(methylamino)-2,7-naphthyridin-3-yl)cyclopropanecarboxamide as yellow solid (105.0 mg, 42.0%). LCMS (ESI) m/z 402.1, [M+H]⁺.

### Step 2: (2)-N-(5-(5-(1-(methoxyamino)ethyl)benzo[d]oxazol-2-yl)-8-(methylamino)-2,7-naphthyridin-3-yl)cyclopropanecarboxamide and (E)-N-(5-(5-(1-(methoxy-amino)ethyl)benzo[d]oxazol-2-yl)-8-(methylamino)-2,7-naphthyridin-3-yl)cyclopropanecarboxamide

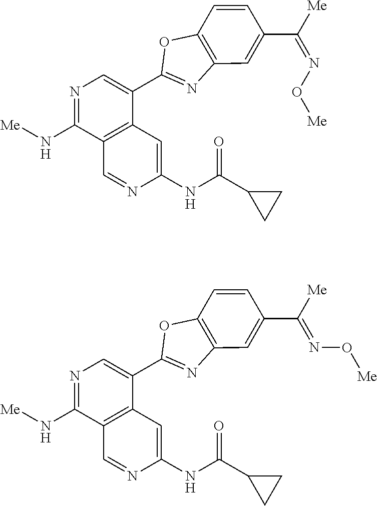 [SMILES: CNc1ncc(-c2nc3cc(/C(C)=N\OC)ccc3o2)c2cc(NC(=O)C3CC3)ncc12 ; CNc1ncc(-c2nc3cc(/C(C)=N/OC)ccc3o2)c2cc(NC(=O)C3CC3)ncc12]

A solution of N-(5-(5-acetylbenzo[d]oxazol-2-yl)-8-(methylamino)-2,7-naphthyridin-3-yl)cyclopropanecarboxamide (90.0 mg; 0.224 mmol; 1.00 eq.) and O-methylhydroxylamine hydrochloride (28.0 mg; 0.335 mmol; 1.50 eq.) in pyridine (4 mL) was stirred at 80° C. for 16 hours under nitrogen atmosphere. The desired product was detected via LCMS. The solvent was concentrated under vacuum. The residue was purified by flash chromatography on pre-packed C18 column using 20-100% of MeOH in water (10 mmol/L NH₄HCO₃) as eluent to provide (Z)—N-(5-(5-(1-(methoxyimino)ethyl)benzo[d]oxazol-2-yl)-8-(methylamino)-2,7-naphthyridin-3-yl)cyclopropanecarboxamide (Example 462) as a yellow solid (4.0 mg, 4.1%) and (E)-N-(5-(5-(1-(methoxyimino)ethyl)benzo[d]oxazol-2-yl)-8-(methylamino)-2,7-naphthyridin-3-yl)cyclopropanecarboxamide (Example 463) as a yellow solid (47.7 mg, 49.4%). LCMS (ESI) m/z 431.2, [M+H]⁺.

HNMR for Example 462: ¹H NMR (400 MHz, DMSO-d₆) δ 11.09 (s, 1H), 9.72 (s, 1H), 9.48 (s, 1H), 8.91 (s, 1H), 8.86-8.82 (m, 1H), 7.90 (s, 1H), 7.77 (d, J=8.4 Hz, 1H), 7.54 (d, J=8.4 Hz, 1H), 3.79 (s, 3H), 3.11 (d, J=4.4 Hz, 3H), 2.24 (s, 3H), 2.16-2.05 (m, 1H), 0.96-0.82 (m, 4H).

HNMR for Example 463: ¹H NMR (400 MHz, DMSO-d₆) δ 11.06 (s, 1H), 9.71 (s, 1H), 9.45 (s, 1H), 8.94 (s, 1H), 8.75-8.67 (m, 1H), 7.98 (s, 1H), 7.81-7.69 (m, 2H), 3.97 (s, 3H), 3.10 (d, J=4.4 Hz, 3H), 2.29 (s, 3H), 2.16-2.06 (m, 1H), 0.97-0.83 (m, 4H).

Examples 464 and 465: Synthesis of N-(5-(5-(1-methyl-1H-1,2,4-triazol-3-yl)benzo[d]oxazol-2-yl)-8-(methylamino)-2,7-naphthyridin-3-yl)cyclopropanecarboxamide (Example 464) and N-(5-(5-(1-methyl-1H-1,2,4-triazol-5-yl)benzo[d]oxazol-2-yl)-8-(methylamino)-2,7-(Example 465)

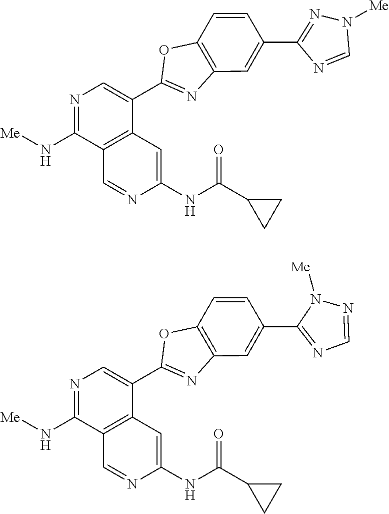 [SMILES: CNc1ncc(-c2nc3cc(-c4ncn(C)n4)ccc3o2)c2cc(NC(=O)C3CC3)ncc12 ; CNc1ncc(-c2nc3cc(-c4ncnn4C)ccc3o2)c2cc(NC(=O)C3CC3)ncc12]

To a stirred solution of N-(5-(5-(1H-1,2,4-triazol-3-yl)benzo[d]oxazol-2-yl)-8-(methylamino)-2,7-naphthyridin-3-yl)cyclopropanecarboxamide (Example 445) (170.0 mg; 0.399 mmol; 1.00 eq.) and K₂CO₃ (110.2 mg; 0.798 mmol; 2.00 eq.) in DMF (2 mL) was added methyl iodide (62.2 mg; 0.439 mmol; 1.10 eq.) dropwise at 0° C. The resulting mixture was stirred at room temperature for 3 hours under nitrogen atmosphere. The reaction was quenched by the addition of saturated sodium bicarbonate solution (3 mL) and extracted with EtOAc (3×10 mL). The combined organic layers were washed with brine (2×10 mL), dried over anhydrous Na₂SO₄. After filtration, the filtrate was concentrated under reduced pressure. The residue was purified by flash chromatography on silica gel column using 0-10% of MeOH in CH₂Cl₂ as eluent to provide N-(5-(5-(1-methyl-1H-1,2,4-triazol-3-yl)benzo[d]oxazol-2-yl)-8-(methylamino)-2,7-naphthyridin-3-yl)cyclopropanecarboxamide (Example 464) as a yellow solid (49.0 mg, 26.4%) and N-(5-(5-(1-methyl-1H-1,2,4-triazol-5-yl)(benzo[d]oxazol-2-yl)-8-(methy amino)-2,7-naphthyridin-3-yl)cyclopropanecarboxamide (Example 465) as an off-white solid (2.2 mg, 1.1%). LCMS (ESI) m/z 441.2, [M+H]⁺.

HNMR for Example 464: ¹H NMR (400 MHz, DMSO-d₆) 11.07 (s, 1H), 9.80 (s, 1H), 9.45 (s, 1H), 8.95 (s, 1H), 8.75-8.65 (m, 1H), 8.57 (s, 1H), 8.27 (d, J=1.6 Hz, 1H), 8.04 (dd, J=8.4, 1.6 Hz, 1H), 7.81 (d, J=8.4 Hz, 1H), 3.96 (s, 3H), 3.10 (d, J=4.4 Hz, 3H), 2.16-2.06 (m, 1H), 1.00-0.81 (m, 4H).

HNMR for Example 465: ¹H NMR (400 MHz, DMSO-d₆) δ 11.07 (s, 1H), 9.68 (s, 1H), 9.46 (s, 1H), 8.97 (s, 1H), 8.80-8.67 (m, 1H), 8.12 (d, J=1.6 Hz, 1H), 8.04 (s, 1H), 7.91 (d, J=8.4 Hz, 1H), 7.81-7.74 (m, 1H), 4.04 (s, 3H), 3.11 (d, J=4.4 Hz, 3H), 2.15-2.03 (m, 1H), 0.96-0.79 (m, 4H).

### Example 466: Synthesis of N-(5-(5-(9-oxa-3,7-diazabicyclo[3.3.1]nonan-3-yl)benzo[d]oxazol-2-yl)-8-(ethylamino)-2,7-naphthyridin-3-yl)cyclopropanecarboxamide

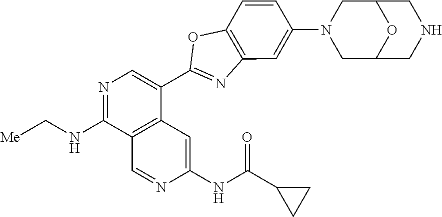 [SMILES: CCNc1ncc(-c2nc3cc(N4CC5CNCC(C4)O5)ccc3o2)c2cc(NC(=O)C3CC3)ncc12]

### Step tert-butyl 7-(2-(6-(cyclopropanecarboxamido)-1-(ethylamino)-2,7-naphthyridin-4-yl)benzo[d]oxazol-5-yl)-9-oxa-3,7-diazabicyclo[3.3.1]nonane-3-carboxylate

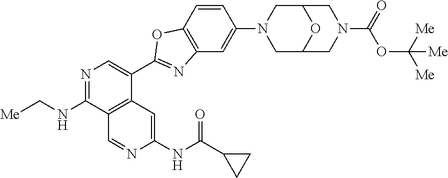 [SMILES: CCNc1ncc(-c2nc3cc(N4CC5CN(C(=O)OC(C)(C)C)CC(C4)O5)ccc3o2)c2cc(NC(=O)C3CC3)ncc12]

A solution of N-(5-(5-bromobenzo[d]oxazol-2-yl)-8-(ethylamino-2,7-naphthyridin-3-yl)cyclopropanecarboxamide (Example 366, Step 3) (150.0 mg; 0.332 mmol; 1.00 eq.), tert-butyl 9-oxa-3,7-diazabicyclo[3.3.1]nonane-3-carboxylate (302.8 mg; 1.326 mmol; 4.00 eq.), EPhos Pd G₄ (60.9 mg; 0.066 mmol; 0.20 eq.), EPhos (35.5 mg; 0.066 mmol; 0.20 eq.), Cs₂CO₃ (432.2 mg; 1.327 mmol; 4.00 eq.) in 1,4-dioxane (10 mL) was stirred at 100° C. for 72 hours under nitrogen atmosphere. The desired product was detected via LCMS. The resulting mixture was concentrated under reduced pressure. The residue was purified by flash chromatography on silica gel column using 5-10% of MeOH in CH₂Cl₂ as eluent to provide tert-butyl 7-(2-(6-(cyclopropanecarboxamido)-1-(ethylamino)-2,7-naphthyridin-4-yl)benzo[d]oxazol-5-yl)-9-oxa-3,7-diazabicyclo[3.3.1]nonane-3-carboxylate as a yellow solid (88.0 mg, 44.2%). LCMS (ESI) m/z 600.0, [M+H]⁺.

### Step 2: N-(5-(5-(9-oxa-3,7-diazabicyclo[3.3.1]nonan-3-yl)benzo[d]oxazol-2-yl)-8-(ethylamino)-2,7-naphthyridin-3-yl)cyclopropanecarboxamide

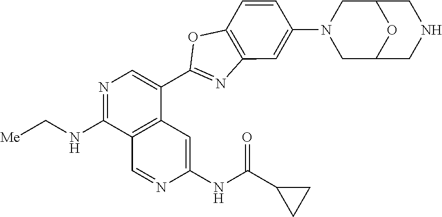 [SMILES: CCNc1ncc(-c2nc3cc(N4CC5CNCC(C4)O5)ccc3o2)c2cc(NC(=O)C3CC3)ncc12]

To a stirred solution of tert-butyl 7-(2-(6-(cyclopropanecarboxamido)-1-(ethylamino)-2,7-naphthyridin-4-yl)benzo[d]oxazol-5-yl)-9-oxa-3,7-diazabicyclo[3.3.1]nonane-3-carboxylate (70.0 mg; 0.117 mmol, 1 eq.) in MeOH (2 mL) was added HCl (gas) (4 M in 1,4-dioxane, 7 mL). The resulting solution was stirred at room temperature for 1 hour. The desired product was detected via. LCMS. The mixture was concentrated under reduced pressure. The residue was purified by flash chromatography on pre-packed C18 column using 50-70% of Maki: in water (10 mmol/L NH₄HCO₃) as eluent to provide N-(5-(5-(9-oxa-3,7-diazabicyclo[3.3.1]nonan-3-yl)benzo[d]oxazol-2-yl)-8-(ethylamino)-2,7-naphthyridin-3-yl)cyclopropanecarboxamide as a yellow solid (48.2 mg, 80.3%). LCMS (ESI) m/z 500.2, [M+H]⁺. ¹H NMR (400 MHz, DMSO-d₆) δ 11.05 (s, 1H), 9.68 (s, 1H), 9.48 (s, 1H), 8.88 (s, 1H), 8.58 (t, J=5.6 Hz, 1H), 7.61 (d, J=8.8 1H), 7.30 (d, J=2.0 Hz, 1H), 7.12 (dd, J=8.8, 2.4 Hz, 1H), 3.85-3.76 (m, 4H), 3.67-3.62 (m, 2H), 3.16-3.10 (m, 7H), 2.16-2.07 (m, 1H), 1.29 (t, J=7.2 Hz, 3H), 0.94-0.84 (m, 4H).

### Examples 467 and 468: Synthesis of (1S,2R)-2-(hydroxymethyl)-N-(5-(5-methoxybenzo[d]oxazol-2-yl)-8-(methylamino)-2,7-naphthyridin-3-yl)cyclopropane-1-carboxamide (Example 467) and (1R,2S)-2-(hydroxy methyl)-N-(5-(5-methoxybenzo[d]oxazol-2-yl)-8-(methylamino)-2,7-naphthyridin-3-yl)cyclopropane-1-carboxamide (Example 468)

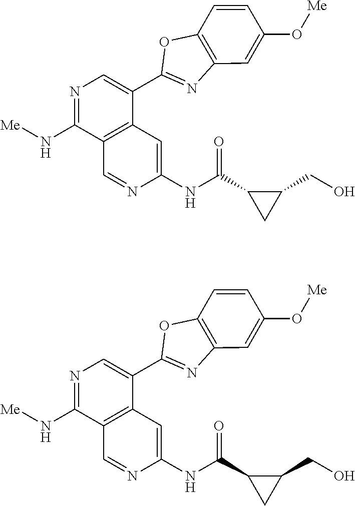 [SMILES: CNc1ncc(-c2nc3cc(OC)ccc3o2)c2cc(NC(=O)[C@H]3C[C@H]3CO)ncc12 ; CNc1ncc(-c2nc3cc(OC)ccc3o2)c2cc(NC(=O)[C@@H]3C[C@@H]3CO)ncc12]

### Step 1: ethyl 2-((trityloxy)methyl)cyclopropane-1-carboxylate (cis racemate)

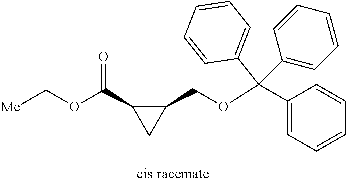 [SMILES: CCOC(=O)[C@@H]1C[C@@H]1COC(c1ccccc1)(c1ccccc1)c1ccccc1]

To a solution of ethyl 2-(hydroxymethyl)cyclopropane-1-carboxylate (cis racemate) (200.0 mg; 1,387 mmol; 1.00 eq.) in CH₂Cl₂ (3 mL) were added (chloromethanetriyl)tribenzene (580.1 mg; 2.081 mmol; 1.50 eq.), DMAP (84.7 mg; 0.693 mmol; 0.50 eq.) and Et₃N (224.6 mg; 2.220 mmol; 1.60 eq.) at room temperature under nitrogen atmosphere. The resulting mixture was stirred at room temperature for 14 hours. The resulting mixture was concentrated under reduced pressure. The residue was purified by flash chromatography on silica gel column using 0-20% of EtOAc in petroleum ether as eluent to provide ethyl 2-((trityloxy)methyl)cyclopropane-1-carboxylate (cis racemate) as a white solid (419.2 mg, 78.1%). LCMS (ESI) m/z 387.2 [M+H]⁺.

### Step 2: 2-((trityloxy)methyl)cyclopropane-1-carboxylic acid (cis racemate)

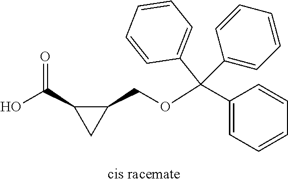 [SMILES: O=C(O)[C@@H]1C[C@@H]1COC(c1ccccc1)(c1ccccc1)c1ccccc1]

To a solution of NaOH (219.9 mg; 5.498 mmol; 5.00 eq.) in a mixture solvent of THF/EtOH/H₂O (1:1:1, 9 mL) was added ethyl 2-((trityloxy)methyl)cyclopropane-1-carboxylate (425.0 mg; 1.100 mmol; 1.00 eq.) at room temperature. The mixture was stirred at 70° C. for 2 hours. The resulting mixture was concentrated under reduced pressure and acidified to pH=4 with 3 M HCl aqueous solution. The aqueous layer was extracted with CH₂Cl₂ (3×40 mL). The combined organic layers were dried over anhydrous Na₂SO₄. After filtration, the filtrate was concentrated under reduced pressure to afford 2-((trityloxy)methyl)cyclopropane-1-carboxylic acid (cis racemate) as a white solid (369.4 mg, 93.7%).

### Step 3: N-(5-(5-methoxybenzo[d]oxazol-2-yl)-8-(methylamino)-2,7-naphthyridin-3-yl)-2-((trityloxy)methyl)cyclopropane-1-carboxamide (cis racemate)

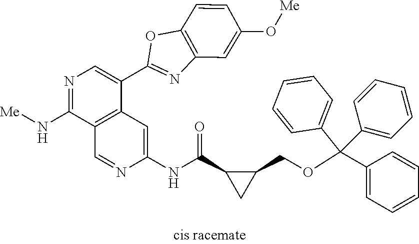 [SMILES: CNc1ncc(-c2nc3cc(OC)ccc3o2)c2cc(NC(=O)[C@@H]3C[C@@H]3COC(c3ccccc3)(c3ccccc3)c3ccccc3)ncc12]

To a stirred solution of 4-(5-methoxybenzo[d]oxazol-2-yl)-N¹-methyl-2,7-naphthyridine-1,6-diamine (Example 372, step 3) (150.0 mg; 0.467 mmol; 1.00 eq.) and 2-((trityloxy)methyl)cyclopropane-1-carboxylic acid (cis racemate) (150.5 mg; 0.420 mmol; 0.90 eq.) in pyridine (4 mL) was added POCl₃ (214.7 mg; 1.400 mmol; 3.00 eq.) dropwise at 0° C. under nitrogen atmosphere. The resulting mixture was stirred at room temperature for 0.5 hour. The desired product was detected via. LCMS. The reaction was quenched by the addition of water (1 mL) at 0° C. The resulting mixture was concentrated under reduced pressure. The residue was purified by flash chromatography on silica gel column using 0-20% of MeOH in CH₂Cl₂ as eluent to provide N-(5-(5-methoxybenzo[d]oxazol-2-yl)-8-(methylamino)-2,7-naphthyridin-3-yl)-2-((trityloxy)methyl)cyclopropane-1-carboxamide (cis racemate) as a yellow solid (185.1 mg, 59.9%). LCMS (ESI) m/z 662.3, [M+H]⁺.

### Step 4: (1S,2R)-2-(hydroxymethyl)-N-(5-(5-methoxybenzo[d]oxazol-2-yl)-8-(methylamino)-2,7-naphthyridin-3-yl)cyclopropane-1-carboxamide and (1R,2S)-2-(hydroxymethyl)-N-(5-(5-methoxybenzo[d]oxazol-2-yl)-8-(methylamino-2,7-naphthyridin-3-yl)cyclopropane-1-carboxamide

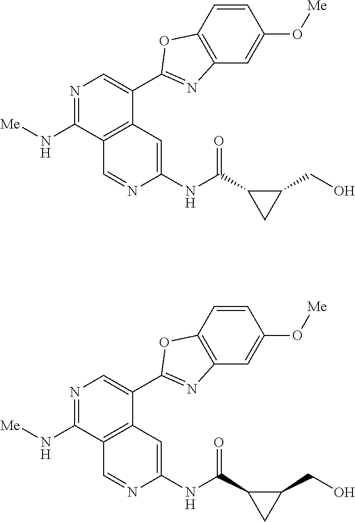 [SMILES: CNc1ncc(-c2nc3cc(OC)ccc3o2)c2cc(NC(=O)[C@H]3C[C@H]3CO)ncc12 ; CNc1ncc(-c2nc3cc(OC)ccc3o2)c2cc(NC(=O)[C@@H]3C[C@@H]3CO)ncc12]

To a stirred solution of N-(5-(5-methoxybenzo[d]oxazol-2-yl)-8-(methylamino)-2,7-naphthyridin-3-yl)-2-((trityloxy)methyl)cyclopropane-1-carboxamide (cis racemate) (180.0 mg; 0.272 mmol; 1.00 eq.) in CH₂Cl₂ (5 mL) was added TFA (0.25 mL) dropwise at room temperature under nitrogen atmosphere. The resulting mixture was stirred at room temperature for 10 minutes. The reaction was quenched by the addition of NaHCO₃ aqueous solution at 0° C. The resulting mixture was extracted with CH₂Cl₂ (3×20 mL). The combined organic layers were concentrated under reduced pressure. The residue was purified by flash chromatography on silica gel column using 0-20% of MeOH in CH₂Cl₂ as eluent to provide the cis racemate. The cis racemate was separated by Prep-Chiral-HPLC (Column: Chiral ART Cellulose-SA, 2×25 cm, 5 μm; Mobile Phase A: Hex (0.5% 2 M NH₃-MeOH), Mobile Phase B: EtOH:DCM=1:1; Flow rate: 20 mL/ruin; Gradient: 30% B to 30% B in 23 min; Wave Length: 220/254 nm; R_{T1}(min): 14.84; R_{T2}(min): 18.25) to afford (1S,2R)-2-(hydroxymethyl)-N-(5-(5-methoxybenzo[d]oxazol-2-yl)-8-(methyl amino)-2,7-naphthyridin-3-yl)cyclopropane-1-carboxamide (Example 467, the faster peak) as a yellow solid (12.0 mg, 10.5%) and (1R,2S)-2-(hydroxymethyl)-N-(5-(5-methoxybenzo[d]oxazol-2-yl)-8-(methylamino)-2,7-naphthyridin-3-yl)cyclopropane-1-carboxamide (Example 468, the slower peak) as a yellow solid (12.9 mg, 11.1%). The two configurations are arbitrarily assigned. LCMS (ESI) m/z 420.2, [M+H]⁺.

HNMR for Example 467: ¹H NMR (400 MHz; DMSO-d₆) δ 10.94 (s, 1H), 9.63 (s, 1H), 9.43 (s, 1H), 8.89 (s, 1H), 8.71-8.60 (m, 1H), 7.63 (d, J=8.8 Hz, 1H), 7.29 (d, J=2.4 Hz, 1H), 6.97 (dd, J=8.8, 2.4 Hz, 1H), 4.52 (s, 1H), 3.86 (s, 3H), 3.73-3.64 (m, 1.14), 3.59-3.49 (m, 1H), 3.09 (d, J=4.4 Hz, 3H), 2.21-2.13 (m, 1H), 1.61-1.42 (m, 1H), 1.08-0.98 (m, 2H).

HNMR for Example 468: ¹H NMR (400 MHz, DMSO-d₆) δ 10.94 (s, 1H), 9.63 (s, 1H), 943 (s, 1H), 8.89 (s, 1H), 8.69-8.60 (m, 1H), 7.63 (d, J=8.8 Hz, 1H), 7.29 (d, J=2.4 Hz, 1H), 6.97 (dd, J=8.8, 2.4 Hz, 1H), 4.52 (s, 1H), 3.86 (s, 3H), 3.71-3.64 (m, 1H), 3.60-3.52 (m, 1H), 3.09 (d, J=4.4 Hz, 3H), 2.22-2.13 (m, 1H), 1.56-1.45 (m, 1H), 1.10-0.98 (m, 2H).

### Example 469: Synthesis of (1S,2R)—N-(5-(5-(3-methoxyazetidin-1-yl)benzo[d]oxazol-2-yl)-8-((methyl-d₃)amino)-2,7-naphthyridin-3-yl)-2-methylcyclopropane-1-carboxamide

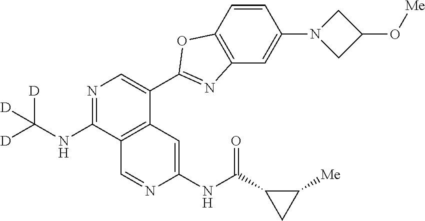 [SMILES: [2H]C([2H])([2H])Nc1ncc(-c2nc3cc(N4CC(OC)C4)ccc3o2)c2cc(NC(=O)[C@H]3C[C@H]3C)ncc12]

### Step 1: N-(5-(5-bromobenzo[d]oxazol-2-yl)-8-methyl-d₃)amino-2,7-naphthyridin-3-yl)cyclopropanecarboxamide

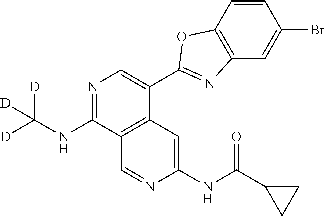 [SMILES: [2H]C([2H])([2H])Nc1ncc(-c2nc3cc(Br)ccc3o2)c2cc(NC(=O)C3CC3)ncc12]

A solution of 2-amino-4-bromophenol (137.6 mg; 0.732 mmol; 1.00 eq.) and N-(5-formyl-8-((methyl-d₃)amino)-2,7-naphthyridin-3-yl)cyclopropanecarboxamide (200.0 mg; 0.732 mmol; 1.00 eq.) and in toluene (8 mL) was stirred at 110° C. for 16 hours. The mixture was concentrated under vacuum. The residue was dissolved in CH₂Cl₂ (8 mL), to this solution was added DDQ (182.7 mg; 0.805 mmol; 1.10 eq.). The resulting mixture was stirred at room temperature for 2 hours. The solvent was concentrated under vacuum. The residue was purified by flash chromatography on pre-packed C18 column using 20-60% of MeCN in water (10 mmol/L NH₄HCO₃) as eluent to provide the crude product. The crude product was purified by flash chromatography on silica gel column using 2-10% of MeOH in CH₂Cl₂ to afford N-(5-(5-bromobenzo[d]oxazol-2-yl)-8-((methyl-d₃)amino)-2,7-naphthyridin-3-yl)cyclopropanecarboxamide as a yellow solid (180.0 mg, 55.74%). LCMS (ESI) m/z 441.0, [M+H]⁺.

### Step 2: N-(5-(5-(3-methoxyazetidin-yl)benzo oxazol-2-yl)-8-((methyl-d₃)amino)-2,7-naphthyridin-3-yl)cyclopropanecarboxamide

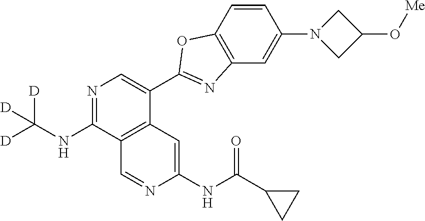 [SMILES: [2H]C([2H])([2H])Nc1ncc(-c2nc3cc(N4CC(OC)C4)ccc3o2)c2cc(NC(=O)C3CC3)ncc12]

A solution of N-(5-(5-bromobenzo[d]oxazol-2-yl)-8-((methyl-d₃)amino)-2,7-naphthyridin-3-yl)cyclopropanecarboxamide (140.0 mg; 0.317 mmol, 1.00 eq), 3-methoxyazetidine hydrochloride (117.6 mg; 0.952 mmol; 3.00 eq), EPhos Pd G₄ (58.3 mg; 0.063 mmol; 0.20 eq), EPhos (33.9 mg; 0.063 mmol; 0.20 eq) and Cs₂CO₃ (516.8 mg; 1.586 mmol; 5.00 eq) in 1,4-dioxane (5 mL) was stirred at 100° C. for 14 hours under nitrogen atmosphere. The desired product was detected via. LCMS. The mixture was concentration under reduced pressure. The residue was purified by flash chromatography on silica gel column using 0-15% of MeOH in CH₂Cl₂ as eluent to afford N-(5-(5-(3-methoxyazetidin-1-yl)(benzo[d]oxazol-2-yl)-8-((methyl-d₃)amino)-2,7-naphthyridin-3-yl)cyclopropanecarboxamide as a yellow solid (120 mg, 84.5%). LCMS (ESI) m/z 448.2, [M+H]⁺.

### Step 3: 445(3-methoxyazetidin-1-yl)benzo[d]oxazol-2-yl)-N¹-(methyl-d₃)-2,7-naphthyridine-4,6-diamine

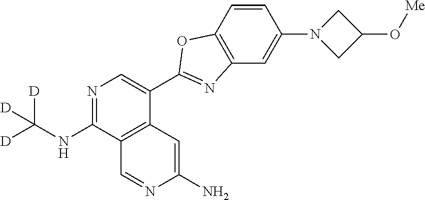 [SMILES: [2H]C([2H])([2H])Nc1ncc(-c2nc3cc(N4CC(OC)C4)ccc3o2)c2cc(N)ncc12]

To a stirred solution of N-(5-(5-(3-methoxyazetidin-1-yl)benzo[d]oxazol-2-yl)-8-((methyl-d₃)amino)-2,7-naphthyridin-3-yl)cyclopropanecarboxamide (123.0 mg; 0.275 mmol; 1.00 eq) in a mixture solvent of THF/DMSO (5/1, 12 mL) was added a solution of NaOH (109.9 mg; 2.748 mmol; 10.00 eq) in H₂O (3 mL). The resulting mixture was stirred at 60° C. overnight. The desired product was detected via LCMS. The THF was removed under reduced pressure. The resulting mixture was diluted with water (50 mL) to precipitate solids. The precipitated solids were collected by filtration and washed with water (3×10 mL). The solids were dried under reduced pressure to afford 4-(5-(3-methoxyazetidin-1-yl)benzo[d]oxazol-2-yl)-N¹-(methyl-d₃)-2,7-naphthyridine-1,6-diamine as a yellow solid (80.0 mg, 68.2%). LCMS (ESI) m/z 380.1, [M+H]⁺.

### Step 4: (1S,2R)—N-(5-(5-(3-methoxyazetidin-1-yl)benzoldioxazol-2-yl)-8-((methyl-d₃)amino)-2,7-naphthyridin-3-yl)-2-methylcyclopropane-1-carboxamide

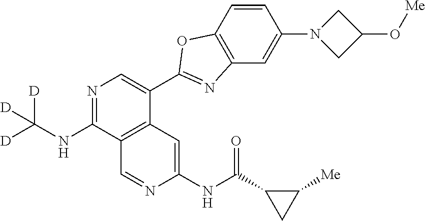 [SMILES: [2H]C([2H])([2H])Nc1ncc(-c2nc3cc(N4CC(OC)C4)ccc3o2)c2cc(NC(=O)[C@H]3C[C@H]3C)ncc12]

(1S,2R)—N-(5-(5-(3-methoxyazetidin-1-yl)benzo[d]oxazol-2-yl)-8-((methyl-d₃)amino)-2,7-naphthyridin-3-yl)-2-methylcyclopropane-1-carboxamide was synthesized using a similar procedure that was previously described in Example 421, by using 4-(5-(3-methoxyazetidin-1-yl)benzo[d]oxazol-2-yl)-N¹-methyl-d₃)-2,7-naphthyridine-1,6-diamine and (1S,2R)-2-methylcyclopropane-1-carboxylic acid as the starting material. LCMS (ESI) m/z 462.2, [M+H]⁺. NMR (400 MHz, DMSO-d₆) δ 10.91 (s, 1H), 9.62 (s, 1H), 9.42 (s, 1H), 8.87 (s, 1H), 8.58 (s, 1H), 7.53 (d, J=8.4 Hz, 1H), 6.72 (d, J=2.4 Hz, 1H), 6.50-6.48 (m, 1H), 4.36-4.33 (m, 1H), 4.13-4.10 (m, 2H), 3.66-3.63 (m, 2H), 3.27 (s, 3H), 2.13-2.08 (m, 1H), 1.33-1.30 (m, 1H), 1.16 (d, 6.0 Hz, 3H), 1.06-0.97 (m, 1H), 0.93-0.85 (m, 1H).

### Example 470: Synthesis of N-(5-(6-methoxy-[1,2,4]triazolo[1,5-a]pyridin-2-yl)-8-(methylamino)-2,7-naphthyridin-3-yl)cyclopropanecarboxamide

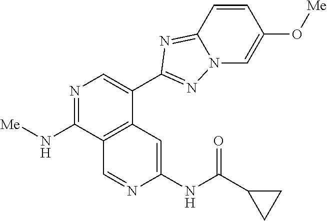 [SMILES: CNc1ncc(-c2nc3ccc(OC)cn3n2)c2cc(NC(=O)C3CC3)ncc12]

### Step 1: O-ethyl N-((5-methoxypyridin-2-yl)carbamothioyl)carbamate

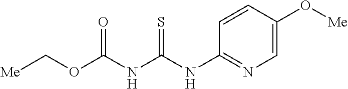 [SMILES: CCOC(=O)NC(=S)Nc1ccc(OC)cn1]

A solution of 5-methoxypyridin-2-amine (10.00 g; 80.55 mmol; 1.00 eq.) and O-ethyl carbonisothiocyanatidate (11.60 g; 88.61 mmol; 1.10 eq.) in 1,4-dioxane (10 mL) was stirred at room temperature overnight under nitrogen atmosphere. The desired product was detected via LCMS. The resulting mixture was concentrated under reduced pressure to afford O-ethyl N-((5-methoxypyridin-2-yl)carbamothioyl)carbamate as a yellow solid (20.00 g, 88.7%). LCMS (ESI) ink 255.2, [M+H]⁺.

### Step 2: 6-methoxy-[1,2,4]triazolo[1,5-a]pyridin-2-amine

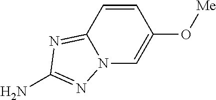 [SMILES: COc1ccc2nc(N)nn2c1]

To a stirred solution of O-ethyl N-((5-methoxypyridin-2-yl)carbamothioyl)carbamate (20.00 g; 78.34 mmol: 1.00 eq) in a mixture solvent of MeOH/EtOH (1:1, 200 mL) were added NH₂OH·HCl (10.80 g, 156.69 mmol; 2.00 eq.) and DIEA (20.20 g; 78.34 mmol; 2.00 eq.) dropwise at room temperature. The resulting mixture was stirred at 70° C. for 4 hours. Upon completion, the resulting mixture was concentrated under reduced pressure. The residue was purified by flash chromatography on silica gel column using 5-8% of MeOH in CH₂Cl₂ as eluent to provide 6-methoxy-[1,2,4]triazolo[1,5-a]pyridin-2-amine as a off-white solid (12.00 g, 93.3%). LCMS (ESI) m/z 165.1, [M+H]⁺.

### Step 3: 2-chloro-6-methyl-[1,2,4]triazolo[1,5-a]pyridine

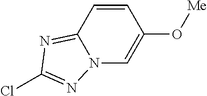 [SMILES: COc1ccc2nc(Cl)nn2c1]

To a stirred solution of 6-methoxy-[1,2,4]triazolo[1,5-a]pyridin-2-amine (12.00 g; 73.1 mmol; 1.00 eq.) and CuCl₂ (2.90 g; 21.6 mmol; 0.30 eq.) in HCl (12 M in water, 120 ML) was added a solution of NaNO₂ (12.00 g; 173.93 mmol; 2.38 eq.) in water (30 mL) at 0° C. under nitrogen atmosphere. The resulting mixture was stirred at room temperature for 1 hour. The desired product was detected via LCMS. The mixture was neutralized to pH=7 with a saturated. Na₂CO₃ aqueous solution. The resulting mixture was extracted with EtOAc (2×200 mL). The combined organic layers were washed with brine (2×100 mL), dried over anhydrous Na₂SO₄, filtered, and concentrated under reduced pressure to afford 2-chloro-6-methoxy-[1,2,4]triazolo[1,5-a]pyridine as a white solid (4.50 g, 33.5%). LCMS (ESI) m/z 184.0, [M+H]⁺.

### Step 4: N-(5-(6-methoxy-[1,2,4]triazolo[1,5-a]pyridin-2-yl)-8-(methylamino)-2,7-naphthyridin-3-yl)cyclopropanecarboxamide

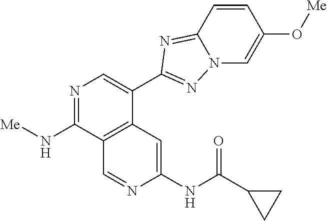 [SMILES: CNc1ncc(-c2nc3ccc(OC)cn3n2)c2cc(NC(=O)C3CC3)ncc12]

To a stirring mixture of N-(8-(methylamino)-5-(4,4,5,5-tetramethyl-1,3,2-dioxaborolan-2-yl)-2,7-naphthyridin-3-yl)cyclopropanecarboxamide (1.00 g; 2.72 mmol; 1.00 eq.) and 2-chloro-6-methoxy-[1,2,4]triazolo[1,5-a]pyridine (250.0 mg; 1.36 mmol; 0.50 eq.) in a mixture solvent of 1,4-dioxane/water (5:1, 12 mL) were added K₃PO₄ (864.0 mg; 4.1 mmol; 1.50 eq.), XPhos Pd G₃ (345.0 mg; 0.41 mmol; 0.15 eq.) and XPhos (194.0 mg; 0.41 mmol; 0.15 eq.). The mixture was stirred at 110° C. for 1 hour under nitrogen atmosphere. Upon completion, the resulting mixture was concentrated under reduced pressure. The residue was purified by flash chromatography on silica gel column using 1-9% of MeOH in CH₂Cl₂ as eluent to provide the crude product. The product was purified by flash chromatography on pre-packed C18 column using 30-70% of MeCN in water (10 mmol/L NH₄HCO₃) as eluent to provide N-(5-(6-methoxy-[1,2,4]triazolo[1,5-a]pyridin-2-yl)-8-(methylamino)-2,7-naphthyridin-3-yl)cyclopropanecarboxamide as a white solid (100.7 mg, 9.5%). LCMS (ESI) m/z 390.2, [M+H]⁺. ¹H NMR (400 MHz, DMSO-d₆) δ 10.92 (s, 1H), 9.41 (s, 2H), 8.73 (s, 1H), 8.67 (d, J=2.0 Hz, 1H), 8.29-8.21 (m, 1H), 7.78 (d, J=9.6 Hz, 1H), 7.46 (dd, J=9.6, 2.0 Hz, 1H), 3.91 (s, 3H), 3.05 (d, J=4.4 Hz, 3H), 2.13-2.02 (m, 1H), 0.91-0.78 (m, 4H).

### Example 471: Synthesis of N-(8-(methylamino)-5-(6-morpholino-[1,2,4]triazolo[1,5-a]pyridin-2-yl)-2,7-naphthyridin-3-yl)cyclopropanecarboxamide

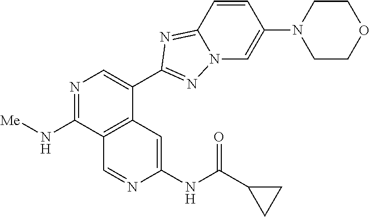 [SMILES: CNc1ncc(-c2nc3ccc(N4CCOCC4)cn3n2)c2cc(NC(=O)C3CC3)ncc12]

### Step 1: 6-bromo-2-chloro-[1,2,4]triazolo[1,5-a]pyridine

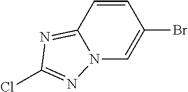 [SMILES: Clc1nc2ccc(Br)cn2n1]

A mixture of 6-bromo-[1,2,4]triazolo[1,5-a]pyridin-2-amine (5.00 g; 23.47 mmol; 1.00 eq.) and CuCl₂ (1.12 g; 17.60 mmol; 0.75 eq.) in HCl (12 Min water, 100 mL). To the above mixture was added dropwise a solution of NaNO₂ (1.94 g; 28.16 mmol; 1.20 eq.) in water (20 mL) over 10 minutes at 0° C. The resulting mixture was stirred at room temperature overnight. The precipitated solids were filtered, washed with water (3×100 mL) to afford 6-bromo-2-chloro-[1,2,4]triazolo[1,5-a]pyridine as a white solid (5.02 g, 81.6%). LCMS (ESI) m/z 231.9, [M+H]⁺.

### Step 2: 4-(2-chloro-[1,2,4]triazolo[1,5-a]pyridin-6-yl)morpholine

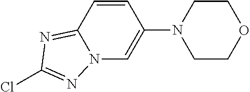 [SMILES: Clc1nc2ccc(N3CCOCC3)cn2n1]

To a stirring mixture of 6-bromo-2-chloro-[1,2,4]triazolo[1,5-a]pyridine (500.0 mg; 2.15 mmol; 1.00 eq.) in 1,4-dioxane (10 mL) was added Pd-PEPPSI-IHeptCl chloropyridine (209.4 mg; 0.215 mmol; 0.10 eq.), Cs₂CO₃ (10.40 g; 4.302 mmol; 2.00 eq.) and morpholine (187.3 mg; 2.151 mmol, 1.00 eq.). The mixture was stirred at 100° C. for 16 hours under nitrogen atmosphere. The desired product was detected via LCMS. The resulting mixture was concentrated under reduced pressure. The residue was purified by flash chromatography via a silica gel column using 10-60% of EtOAc in petroleum ether as eluent to provide 4-(2-chloro-[1,2,4]triazolo[1,5-a]pyridin-6-yl) morpholine as a white solid (320.0 mg, 62.3%). LCMS (ESI) m/z 2391, [M+H]⁺.

Each intermediate in Table 17 below was prepared using a similar experimental procedure to prepare 4-(2-chloro[1,2,4]triazolo[1,5-a]pyridin-6-yl)morpholine, where morpholine was replaced with the reagent as shown Table 17 below.

TABLE 17

| Intermediate | Reagent | MS |
|---|---|---|
| 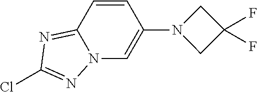 [SMILES: FC1(F)CN(c2ccc3nc(Cl)nn3c2)C1] | 3,3-difluoroazetidine hydrochloride | LCMS (ESI) m/z 245.0, [M + H ]⁺. |
| 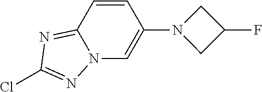 [SMILES: FC1CN(c2ccc3nc(Cl)nn3c2)C1] | 3-fluoroazetidine | LCMS (ESI) m/z 227.0, [M + H]⁺. |
| 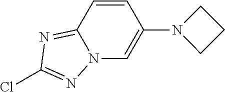 [SMILES: Clc1nc2ccc(N3CCC3)cn2n1] | azetidine | LCMS (ESI) m/z 209.1, [M + H]⁺. |
| 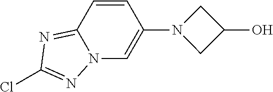 [SMILES: OC1CN(c2ccc3nc(Cl)nn3c2)C1] | azetidin-3-ol hydrochloride | LCMS (ESI) m/z 225.0, [M + H]⁺. |
| 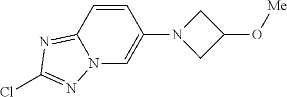 [SMILES: COC1CN(c2ccc3nc(Cl)nn3c2)C1] | 3-methoxyazetidine hydrochloride | LCMS (ESI) m/z 239.1, [M + H]⁺. |
| 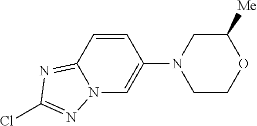 [SMILES: C[C@@H]1CN(c2ccc3nc(Cl)nn3c2)CCO1] | (2R)-2-methylmorpholine | LCMS (ESI) m/z 253.1, [M + H]⁺. |
| 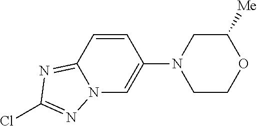 [SMILES: C[C@H]1CN(c2ccc3nc(Cl)nn3c2)CCO1] | (S)-2-methylmorpholine | LCMS (ESI) m/z 253.1 [M + H]⁺. |
| 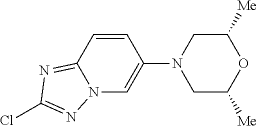 [SMILES: C[C@@H]1CN(c2ccc3nc(Cl)nn3c2)C[C@H](C)O1] | (2R,6S)-2,6- dimethylmorpholine | LCMS (ESI) m/z 267.1, [M + H]⁺. |
| 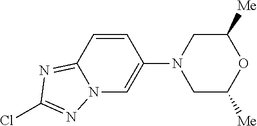 [SMILES: C[C@@H]1CN(c2ccc3nc(Cl)nn3c2)C[C@@H](C)O1] | 2,6-dimethylmorpholine (trans racemate) | LCMS (ESI) m/z 267.1, [M + H]⁺. |
| 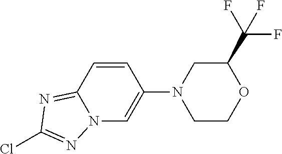 [SMILES: FC(F)(F)[C@@H]1CN(c2ccc3nc(Cl)nn3c2)CCO1] | (S)-2- (trifluoromethyl)morpholine hydrochloride | LCMS (ESI) m/z 307.0, [M + H]⁺. |
| 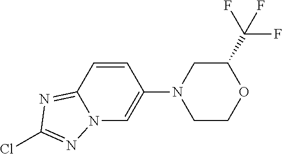 [SMILES: FC(F)(F)[C@H]1CN(c2ccc3nc(Cl)nn3c2)CCO1] | (R)-2- (trifluoromethyl)morpholine hydrochloride | LCMS (ESI) m/z 307.0, [M + H]⁺. |
| 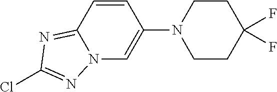 [SMILES: FC1(F)CCN(c2ccc3nc(Cl)nn3c2)CC1] | 4,4-difluoropiperidine | LCMS (ESI) m/z 273.1, [M + H]⁺. |
| 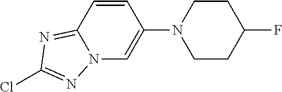 [SMILES: FC1CCN(c2ccc3nc(Cl)nn3c2)CC1] | 4-fluoropiperidine hydrochloride | LCMS (ESI) m/z 255.1, [M + H]⁺. |
| 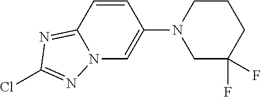 [SMILES: FC1(F)CCCN(c2ccc3nc(Cl)nn3c2)C1] | 3,3-difluoropiperidine hydrochloride | LCMS (ESI) m/z 273.1, [M + H]⁺. |
| 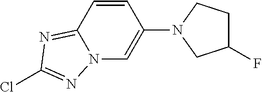 [SMILES: FC1CCN(c2ccc3nc(Cl)nn3c2)C1] | 3-fluoropyrrolidine hydrochloride | LCMS (ESI) m/z 241.1, [M + H]⁺. |
| 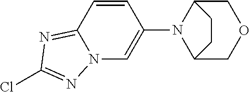 [SMILES: Clc1nc2ccc(N3C4CCC3COC4)cn2n1] | 3-oxa-8- azabicyclo[3.2.1]octane hydrochloride | LCMS (ESI) m/z 265.1, [M + H]⁺. |
| 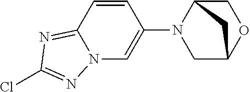 [SMILES: Clc1nc2ccc(N3C[C@@H]4C[C@H]3CO4)cn2n1] | (1S,4S)-2-oxa-5- azabicyclo[2.2.1]heptane hydrochloride | LCMS (ESI) m/z 251.1, [M + H]⁺. |
| 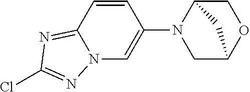 [SMILES: Clc1nc2ccc(N3C[C@H]4C[C@@H]3CO4)cn2n1] | (1R,4R)-2-oxa-5- azabicyclo[2.2.1]heptane hydrochloride | LCMS (ESI) m/z 251.1, [M + H]⁺. |

### Step 3: N-(8-(methylamino)-5-(6-morpholino-[1,2,4]triazolo[1,5-a]pyridin-2-yl)2,7-naphthyridin-3-yl)cyclopropanecarboxamide

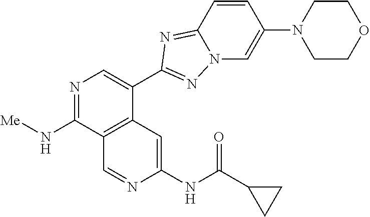 [SMILES: CNc1ncc(-c2nc3ccc(N4CCOCC4)cn3n2)c2cc(NC(=O)C3CC3)ncc12]

To a stirring mixture of N-(8-(methylamino)-5-(4,5,5-tetramethyl-1,3,2-dioxaborolan-2-yl)-2,7-naphthyridin-3-yl)cyclopropanecarboxamide (500.0 mg; 1.36 mmol; 1.00 eq.) in a mixture solvent of 1,4-dioxane/water (0.5:1, 12 mL) was added XPhos (194.1 mg; 0.41 mmol; 0.30 eq.), XPhos Pd. G₃ (344.8 mg; 0.41 mmol; 0.30 eq.), K₃PO₄(864.6 mg; 4.07 mmol; 3.00 eq.) and 4-(2-chloro-[1.2.4]triazolo[1,5-a]pyridin-6-yl)morpholine (320.0 mg; 1.36 mmol; 1.00 eq.). The mixture was stirred at 110° C. for 1 hour under nitrogen atmosphere. The desired product was detected via LCMS. The solvent was concentrated under vacuum The residue purified by flash chromatography on silica gel column using 2-10% of MeOH in CH₂Cl₂ as eluent to afford N-(8-(methylamino)-5-(6-morpholino-[1,2,4]triazolo[1,5-a]pyridin-2-yl)-2,7-naphthyridin-3-yl)cyclopropanecarboxamide as a yellow solid (211.4 mg, 35.0%). LCMS (ESI) m/z 445.2, [M+H]⁺. ¹H NMR (400 MHz, DMSO-d₆) δ 10.91 (s, 1H), 9.40 (s, 2H), 8.72 (s, 1H), 8.33 (d, J=2.0 Hz, 1H), 8.26-8.20 (m, 1H), 7.74 (d, J=9.6 Hz, 1H), 7.67 (dd, J=9.6, 2.0 Hz, 1H), 3.83-3.76 (m, 4H), 3.20-3.13 (m, 4H), 3.05 (d, J=4.4 Hz, 3H), 2.12-2.02 (m, 1H), 0.91-0.78 (m, 4H).

### Examples 472-489

Each compound in Table 18 below was prepared using a similar experimental procedure to prepare Example 471, using N-(8-(methylamino)-5-(4,4,5,5-tetramethyl-1,3,2-dioxaborolan-2-yl)-2,7-naphthyridin-3-yl)cyclopropanecarboxamide as the common intermediate and appropriate 2-chloro-[1,2,4]triazolo[1,5-a]pyridine substrates.

TABLE 18

| Example | Structure and Name | MS and ¹H NMR |
|---|---|---|
| Example 472 | 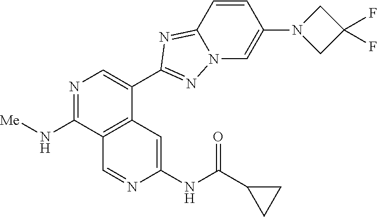 | LCMS (ESI) m/z 451.2, [M + H]⁺. ¹H NMR (400 MHz, DMSO-d₆) δ 10.92 (s, 1H), 9.41 (s, 1H), 9.40 (s, 1H), 8.73 (s, 1H), 8.30 (d, J = 2.0 Hz, 1H), 8.26-8.22 (m, 1H), 7.78 (d, J = 9.2 Hz, 1H), 7.25 (dd, J = 9.2, 2.0 Hz, 1H), 4.45-4.33 (m, 4H), 3.05 (d, J = 4.4 Hz, 3H), 2.10-2.04 (m, 1H), 0.80- 0.80 (m, 4H). |
| | N-(5-(6-(3,3-difluoroazetidin-1- | |
| | yl)-[1,2,4]triazolo[1,5-a]pyridin- | |
| | 2-yl)-8-(methylamino)-2,7- | |
| | naphthyridin-3- | |
| | yl)cyclopropanecarboxamide | |
| Example 473 | 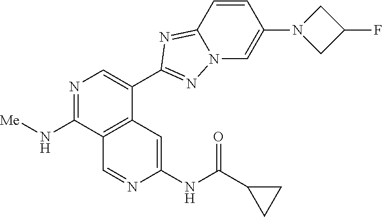 | LCMS (ESI) m/z 433.2, [M + H]⁺. ¹H NMR (400 MHz, DMSO-d₆) δ 10.96 (s, 1H), 9.42 (s, 1H), 9.41 (s, 1H), 8.70 (s, 1H), 8.40-8.23 (m, 1H), 8.16 (d, J = 2.0 Hz, 1H), 7.75 (d, J = 9.6 Hz, 1H), 7.20 (dd, J = 9.6, 2.0 Hz, 1H), 5.62-5.41 (m, 1H), 4.32-4.18 (m, 2H), 4.06-3.93 (m, 2H), 3.05 (d, J = 4.4 Hz, 3H), 2.13-2.02 (m, 1H), 0.90-0.78 (m, 4H). |
| | N-(5-(6-(3-fluoroazetidin-1-yl)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |
| Example 474 | 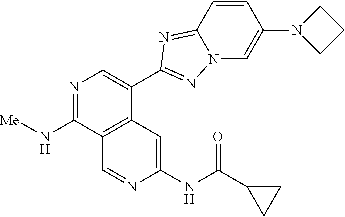 | LCMS (ESI) m/z 415.2, [M + H]⁺. ¹H NMR (400 MHz, DMSO-d₆) δ 10.92 (s, 1H), 9.41 (s, 1H), 9.39 (s, 1H), 8.71 (s, 1H), 8.26-8.19 (m, 1H), 8.05 (d, J = 2.0 Hz, 1H), 7.71 (d, J = 9.6 Hz, 1H), 7.13 (dd, J = 9.6, 2.0 Hz, 1H), 3.96-3.84 (m, 4H), 3.05 (d, J = 4.4 Hz, 3H), 2.41-2.29 (m, 2H), 2.11- 2.03 (m, 1H), 0.91-0.79 (m, 4H). |
| | N-(5-(6-(azetidin-1-yl)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |
| Example 475 | 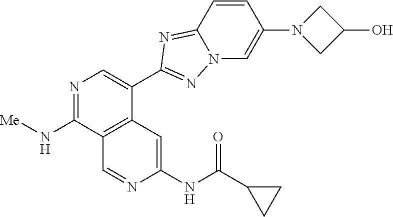 | LCMS (ESI) m/z 431.2, [M + H]⁺. ¹H NMR (400 MHz, DMSO-d₆) δ 10.92 (s, 1H), 9.40 (s, 1H), 9.39 (s, 1H), 8.70 (s, 1H), 8.26-8.20 (m, 1H), 8.05 (d, J = 2.0 Hz, 1H), 7.70 (d, J = 9.6 Hz, 1H), 7.16 (dd, J = 9.6, 2.0 Hz, 1H), 5.68 (d, J = 6.4 Hz, 1H), 4.66- 4.54 (m, 1H), 4.20-4.12 (m, 2H), 3.63-3.55 (m, 2H), 3.05 (d, J = 4.4 Hz, 3H), 2.12-2.02 (m, 1H), 0.90- 0.78 (m, 4H). |
| | N-(5-(6-(3-hydroxyazetidin-1-yl)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |
| Example 476 | 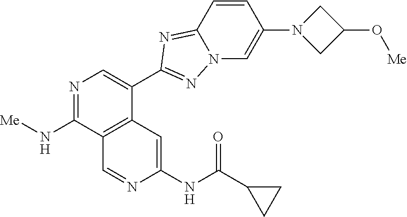 | LCMS (ESI) m/z 445.2, [M + H]⁺. ¹H NMR (400 MHz, DMSO-d₆) δ 10.94 (s, 1H), 9.42 (s, 1H), 9.40 (s, 1H), 8.70 (s, 1H), 8.36-8.19 (m, 1H), 8.06 (d, J = 2.0 Hz, 1H), 7.72 (d, J = 9.6 Hz, 1H), 7.16 (dd, J = 9.6, 2.0 Hz, 1H), 4.40-4.30 (m, 1H), 4.18-4.09 (m, 2H), 3.75-3.65 (m, 2H), 3.27 (s, 3H), 3.05 (d, J = 4.4 Hz, 3H), 2.12- 2.00 (m, 1H), 0.91-0.78 (m, 4H). |
| | N-(5-(6-(3-methoxyazetidin-1-yl)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |
| Example 477 | 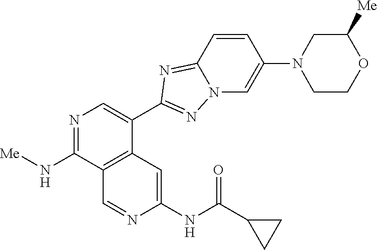 | LCMS (ESI) m/z 459.2, [M + H]⁺. ¹H NMR (400 MHz, DMSO-d₆) δ 10.89 (s, 1H), 9.41 (s, 1H), 9.40 (s, 1H), 8.70 (s, 1H), 8.48-8.11 (m, 2H), 7.74 (d, J = 9.6 Hz, 1H), 7.68 (dd, J = 9.6, 2.0 Hz, 1H), 3.99-3.89 (m, 1H), 3.78- 3.57 (m, 3H), 3.54-3.46 (m, 1H), 3.05 (d, J = 4.4 Hz, 3H), 2.79-2.68 (m, 1H), 2.47-2.37 (m, 1H), 2.13- 1.96 (m, 1H), 1.18 (d, J = 6.9 Hz, 3H), 0.91-0.78 (m, 4H). |
| | (R)-N-(8-(methylamino)-5-(6-(2- | |
| | methylmorpholino)- | |
| | [1,2,4]triazolo[1,5-a]pyridin-2- | |
| | yl)-2,7-naphthyridin-3- | |
| | yl)cyclopropanecarboxamide | |
| Example 478 | 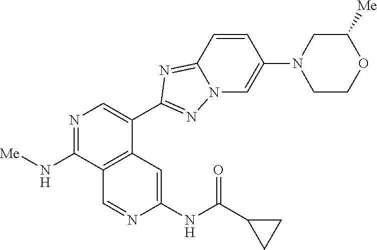 | LCMS (ESI) m/z 459.2, [M + H]⁺. ¹H NMR (400 MHz, DMSO-d₆) δ 10.93 (s, 1H), 9.40 (s, 1H), 9.39 (s, 1H), 8.72 (s, 1H), 8.33 (d, J = 2.0 Hz, 1H), 8.30-8.20 (m, 1H), 7.74 (d, J = 9.6 Hz, 1H), 7.68 (dd, J = 9.6, 2.0 Hz, 1H), 4.00-3.89 (m, 1H), 3.78-3.57 (m, 3H), 3.54-3.47 (m, 1H), 3.05 (d, J = 4.4 Hz, 3H), 2.79-2.67 (m, 1H), 2.48-2.38 (m, 1H), 2.10-2.01 (m, 1H), 1.18 (d, J = 6.0 Hz, 3H), 0.92- 0.80 (m, 4H). |
| | (S)-N-(8-(methylamino)-5-(6-(2- | |
| | methylmorpholino)- | |
| | [1,2,4]triazolo[1,5-a]pyridin-2- | |
| | yl)-2,7-naphthyridin-3- | |
| | yl)cyclopropanecarboxamide | |
| Example 479 | 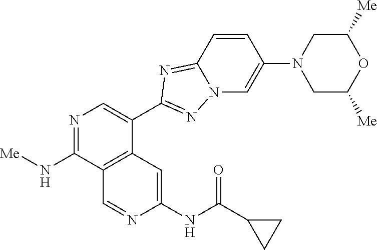 | LCMS (ESI) m/z 473.2, [M + H]⁺. ¹H NMR (400 MHz, DMSO-d₆) δ 10.92 (s, 1H), 9.40 (s, 1H), 9.38 (s, 1H), 8.71 (s, 1H), 8.32 (d, J = 2.0 Hz, 1H), 8.27-8.20 (m, 1H), 7.73 (d, J = 9.6 Hz, 1H), 7.69 (dd, J = 9.6, 2.0 Hz, 1H), 3.82-3.70 (m, 2H), 3.65-3.56 (m, 2H), 3.05 (d, J = 4.4 Hz, 3H), 2.38-2.28 (m, 2H), 2.12-2.02 (m, 1H), 1.18 (d, J = 6.4 Hz, 6H), 0.91-0.78 (m, 4H). |
| | N-(5-(6-((2R,6S)-2,6- | |
| | dimethylmorpholino)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |
| Example 480 | 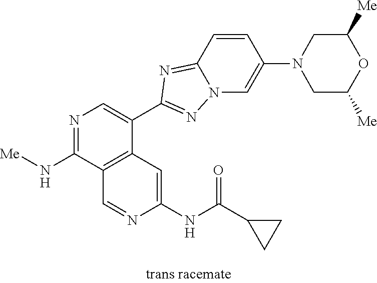 | LCMS (ESI) m/z 473.2, [M + H]⁺. ¹H NMR (400 MHz, DMSO-d₆) δ 10.91 (s, 1H), 9.40 (s, 2H), 8.73 (s, 1H), 8.31 (d, J = 2.0 Hz, 1H), 8.27-8.21 (m, 1H), 7.73 (d, J = 9.6 Hz, 1H), 7.65 (d, J = 9.6, 2.0 Hz, 1H), 4.17-4.05 (m, 2H), 3.24-3.18 (m, 2H), 3.05 (d, J = 4.4 Hz, 3H), 2.92-2.86 (m, 2H), 2.10-2.03 (m, 1H), 1.25 (d, J = 6.4 Hz, 6H), 0.90-0.80 (m, 4H). |
| | N-(5-(6-(2,6- | |
| | dimethylmorpholino)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |
| | (trans racemate) | |
| Example 481 | 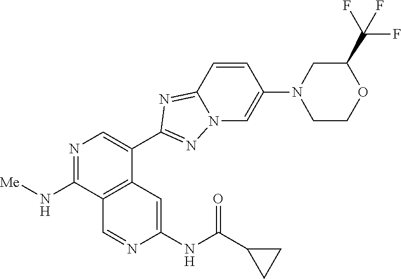 | LCMS (ESI) m/z 513.2, [M + H]⁺. ¹H NMR (400 MHz, DMSO-d₆) δ 10.91 (s, 1H), 9.40 (s, 1H), 9.38 (s, 1H), 8.72 (s, 1H), 8.55-8.47 (m, 1H), 8.27- 8.22 (m, 1H), 7.81-7.71 (m, 2H), 4.45-4.36 (m, 1H), 4.16-4.09 (m, 1H), 3.90-3.71 (m, 2H), 3.62-3.55 (m, 1H), 3.05 (d, J = 4.4 Hz, 3H), 2.95-2.79 (m, 2H), 2.09-2.00 (m, 1H), 0.90-0.80 (m, 4H). |
| | (S)-N-(8-(methylamino)-5-(6-(2- | |
| | (trifluoromethyl)morpholino)- | |
| | [1,2,4]triazolo[1,5-a]pyridin-2- | |
| | yl)-2,7-naphthyridin-3- | |
| | yl)cyclopropanecarboxamide | |
| Example 482 | 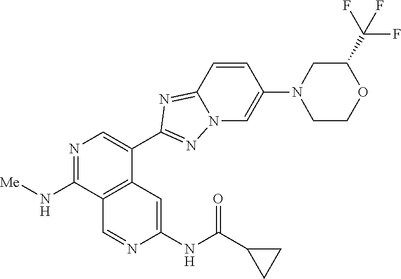 | LCMS (ESI) m/z 513.2, [M + H]⁺. ¹H NMR (400 MHz, DMSO-d₆) δ 10.91 (s, 1H), 9.40 (s, 1H), 9.38 (s, 1H), 8.72 (s, 1H), 8.55-8.47 (m, 1H), 8.27- 8.22 (m, 1H), 7.81-7.71 (m, 2H), 4.45-4.36 (m, 1H), 4.16-4.09 (m, 1H), 3.90-3.71 (m, 2H), 3.62-3.55 (m, 1H), 3.05 (d, J = 4.4 Hz, 3H), 2.95- 2.79 (m, 2H), 2.09-2.00 (m, 1H), 0.90-0.80 (m, 4H). |
| | (R)-N-(8-(methylamino)-5-(6-(2- | |
| | (trifluoromethyl)morpholino)- | |
| | [1,2,4]triazolo[1,5-a]pyridin-2- | |
| | yl)-2,7-naphthyridin-3- | |
| | yl)cyclopropanecarboxamide | |
| Example 483 | 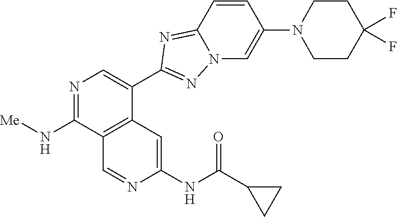 | LCMS (ESI) m/z 479.2, [M + H]⁺. ¹H NMR (400 MHz, DMSO-d₆) δ 10.92 (s, 1H), 9.40 (s, 1H), 9.38 (s, 1H), 8.70 (s, 1H), 8.45 (d, J = 2.0 Hz, 1H), 8.28-8.20 (m, 1H), 7.74 (d, J = 9.6 Hz, 1H), 7.70 (dd, J = 9.6, 2.0 Hz, 1H), 3.36-3.32 (m, 4H), 3.04 (d, J = 4.4 Hz, 3H), 2.19-2.05 (m, 5H), 0.90- 0.80 (m, 4H). |
| | N-(5-(6-(4,4-difluoropiperidin-1- | |
| | yl)-[1,2,4]triazolo[1,5-a]pyridin- | |
| | 2-yl)-8-(methylamino)-2,7- | |
| | naphthyridin-3- | |
| | yl)cyclopropanecarboxamide | |
| Example 484 | 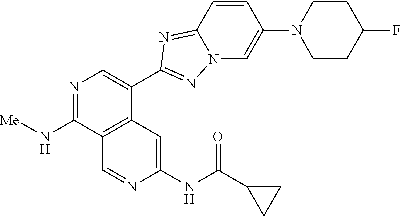 | LCMS (ESI) m/z 461.2, [M + H]⁺. ¹H NMR (400 MHz, DMSO-d₆) δ 10.92 (s, 1H), 9.40 (s, 1H), 9.39 (s, 1H), 8.70 (s, 1H), 8.37 (d, J = 2.0 Hz, 1H), 8.28-8.20 (m, 1H), 7.72 (d, J = 9.6 Hz, 1H), 7.68 (dd, J = 9.6, 2.0 Hz, 1H), 4.94-4.81 (m, 1H), 3.39-3.28 (m, 2H), 3.25-3.12 (m, 2H), 3.04 (d, J = 4.4 Hz, 3H), 2.1-1.98 (m, 3H), 1.93-1.91 (m, 2H), 0.90-0.78 (m, 4H). |
| | N-(5-(6-(4-fluoropiperidin-1-yl)- | |
| | [1,2,4]triazolo[1,5-a]pyridin-2- | |
| | yl)-8-(methylamino)-2,7- naphthyridin-3- | |
| | yl)cyclopropanecarboxamide | |
| Example 485 | 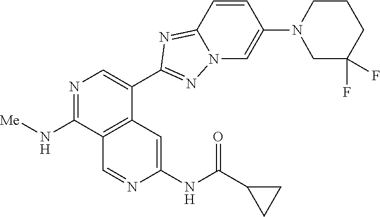 | LCMS (ESI) m/z 479.2, [M + H]⁺. ¹H NMR (400 MHz, DMSO-d₆) δ 10.92 (s, 1H), 9.40 (s, 1H), 9.38 (s, 1H), 8.71 (s, 1H), 8.45 (d, J = 2.0 Hz, 1H), 8.30-8.20 (m, 1H), 7.77-7.66 (m, 2H), 3.57-3.47 (m, 2H), 3.31-3.24 (m, 2H), 3.05 (d, J = 4.4 Hz, 3H), 2.14-2.00 (m, 3H), 1.90-1.81 (m, 2H), 0.89-0.78 (m, 4H). |
| | N-(5-(6-(3,3-difluoropiperidin-1- | |
| | yl)-[1,2,4]triazolo[1,5-a]pyridin- | |
| | 2-yl)-8-(methylamino)-2,7- | |
| | naphthyridin-3- | |
| | yl)cyclopropanecarboxamide | |
| Example 486 | 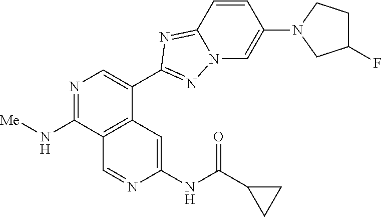 | LCMS (ESI) m/z 447.2, [M + H]⁺. ¹H NMR (400 MHz, DMSO-d₆) δ 10.92 (s, 1H), 9.43 (s, 1H), 9.40 (s, 1H), 8.71 (s, 1H), 8.26-8.20 (m, 1H), 8.11 (d, J = 2.0 Hz, 1H), 7.73 (d, J = 9.6 Hz, 1H), 7.39 (dd, J = 9.6, 2.0 Hz, 1H), 5.57-5.43 (m, 1H), 3.67-3.55 (m, 2H), 3.47-3.44 (m, 2H), 3.04 (d, J = 4.4 Hz, 3H), 2.33-2.20 (m, 2H), 2.10-2.04 (m, 1H), 0.88-0.80 (m, 4H). |
| | N-(5-(6-(3-fluoropyrrolidin-1-yl)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |
| Example 487 | 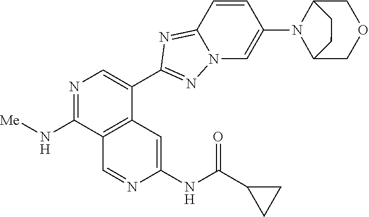 | LCMS (ESI) m/z 471.2, [M + H]⁺. ¹H NMR (400 MHz, DMSO-d₆) δ 10.92 (s, 1H), 9.40 (s, 1H), 9.36 (s, 1H), 8.68 (s, 1H), 8.46 (d, J = 2.0 Hz, 1H), 8.25-8.19 (m, 1H), 7.73 (d, J = 9.6 Hz, 1H), 7.58 (dd, J = 9.6, 2.0 Hz, 1H), 4.25-4.21 (m, 2H), 3.83-3.75 (m, 2H), 3.52-3.45 (m, 2H), 3.05 (d, J = 4.4 Hz, 3H), 2.10-2.03 (m, 1H), 2.00-1.90 (m, 4H), 0.87-0.81 (m, 4H). |
| | N-(5-(6-(3-oxa-8- | |
| | azabicyclo[3.2.1]octan-8-yl)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |
| Example 488 | 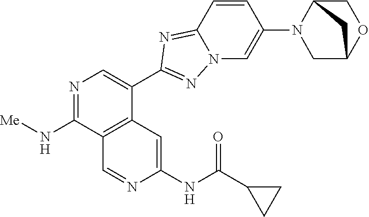 | LCMS (ESI) m/z 457.2, [M + H]⁺. ¹H NMR (400 MHz, DMSO-d₆) δ 10.89 (s, 1H), 9.40 (s, 1H), 9.39 (s, 1H), 8.69 (s, 1H), 8.24 (d, J = 2.4 Hz, 1H), 8.23-8.18 (m, 1H), 7.71 (d, J = 9.6 Hz, 1H), 7.40 (dd, J = 9.6, 2.4 Hz, 1H), 4.72-4.62 (m, 2H), 3.77 (s, 2H), 3.62-3.53 (m, 1H), 3.05 (d, J = 4.4 Hz, 3H), 3.04-2.99 (m, 1H), 2.12- 1.85 (m, 3H), 0.93-0.77 (m, 4H). |
| | N-(5-(6-((1S,4S)-2-oxa-5- | |
| | azabicyclo[2.2.1]heptan-5-yl)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |
| Example 489 | 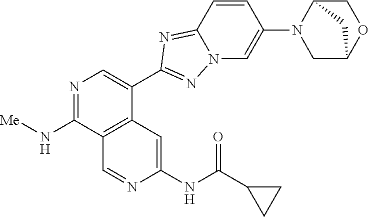 | LCMS (ESI) m/z 457.2, [M + H]⁺. ¹H NMR (400 MHz, DMSO-d₆) δ 10.91 (s, 1H), 9.40 (s, 1H), 9.39 (s, 1H), 8.69 (s, 1H), 8.24 (d, J = 2.0 Hz, 1H), 8.23-8.18 (m, 1H), 7.72 (d, J = 9.6 Hz, 1H), 7.40 (dd, J = 9.6, 2.0 Hz, 1H), 4.71-4.64 (m, 2H), 3.79-3.75 (m, 2H), 3.62-3.55 (m, 1H), 3.09- 3.02 (m, 1H), 3.05 (d, J = 4.4 Hz, 3H), 2.11-2.02 (m, 1H), 1.99-1.86 (m, 1H), 1.93-1.85 (m, 1H), 0.89-0.78 (m, 4H). |
| | N-(5-(6-((1R,4R)-2-oxa-5- | |
| | azabicyclo[2.2.1]heptan-5-yl)- | |
| | [1,2,4]triazolo[1,5-a]pyridin-2- | |
| | yl)-8-(methylamino)-2,7- | |
| | naphthyridin-3- | |
| | yl)cyclopropanecarboxamide | |

### Example 490: Synthesis of N-(5-(5-chloro-[1,2,4]triazolo[1,5-a]pyridin-2-yl)-8-(methylamino)-2,7-naphthyridin-3-yl)cyclopropanecarboxamide

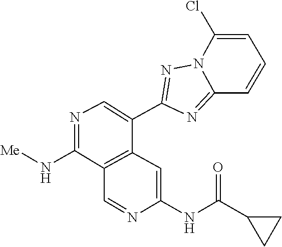 [SMILES: CNc1ncc(-c2nc3cccc(Cl)n3n2)c2cc(NC(=O)C3CC3)ncc12]

### Step 1: 2-bromo-5-chloro-[1,2,4]triazolo[1,5-a]pyridine

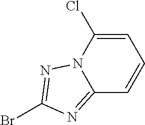 [SMILES: Clc1cccc2nc(Br)nn12]

To a solution of t-BuNO₂ (459.8 trig; 4.459 mmol; 1.50 eq.) in MeCN (5 mL) was added CuBr (638.4 mg; 4.450 mmol; 1.50 eq.). After stirring at 60° C. for 10 minutes, to this mixture was added a solution of 5-chloro-[1,2,4]triazolo[1,5-a]pyridin-2-amine (500.0 mg; 2.966 mmol; 1.00 eq.) in MeCN (10 mL) dropwise at room temperature. The resulting mixture was stirred at 60° C. for 1 hour. The desired product was detected via LCMS. The mixture was allowed to cool down to room temperature. The resulting mixture was diluted with water (50 mL) and extracted with EtOAc (3×20 mL). The combined organic layers were washed with brine (3×10 mL), dried over anhydrous Na₂SO₄, filtered, and concentrated under reduced pressure. The residue was purified by flash chromatography, on silica gel column, using 20-40% of EtOAc in petroleum ether as eluent to provide 2-bromo-5-chloro-[1,2,4]triazolo[1,5-a]pyridine as a yellow solid (390.0 mg, 56.5%). LCMS (ESI) m/z 231.9, [M+H]⁺.

### Step 2: N-(5-(5-chloro[1,2,4]triazolo[1,5-a]pyridin-2-yl)-8-(methylamino))-2,7-naphthyridin-3-yl)cyclopropanecarboxamide

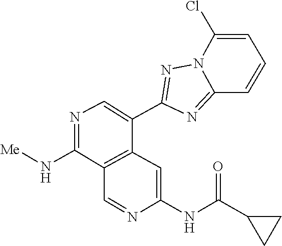 [SMILES: CNc1ncc(-c2nc3cccc(Cl)n3n2)c2cc(NC(=O)C3CC3)ncc12]

To a stirring mixture of 2-bromo-5-chloro-[1,2,4]triazolo[1,5-c]pyridine (350.0 mg; 1,506 mmol; 1.00 eq.) in 1,4-dioxane/water (5:1, 18 mL) were added N-(8-(methylamino)-5-(4,4,5,5-tetramethyl-1,3,2-dioxaborolan-2-yl)-2,7-naphthyridin-3-yl)cyclopropanecarboxamide (900.0 mg; 2,444 mmol; 1.62 eq.). To the above mixture were added Pd(dppf)C12. CH₂Cl₂ (123.3 mg; 0.151 mmol; 0.10 eq.) and CsF (460.0 mg; 3.028 mmol; 2.01 eq.). The resulting solution was stirred at 110° C. overnight under nitrogen atmosphere. Upon completion, the mixture was allowed to cool down to room temperature. The resulting mixture was concentrated under reduced pressure. The residue was purified by flash chromatography on silica gel column using 1-6% of MeOH in CH₂Cl₂ as eluent to provide the crude product. The crude product was purified by Prep-Achiral-SFC (Column: DAICEL DCpak P4VP, 3×25 cm, 5 μm Mobile Phase A: CO₂, Mobile Phase B: Mani (0.1% 2 M-NH₃-MeOH); Flow rate: 60 ml/min; Gradient: isocratic 35% B) to provide N-(5-(5-chloro-[1,2,4]triazolo[1,5-a]pyridin-2-yl)-8-(methylamino)-2,7-naphthyridin-3-yl)cyclopropanecarboxamide as an off-white solid (6.3 mg, 1.1%). LCMS (ESI) m/z 394.1, [M+H]⁺. ¹H NMR (400 MHz, DMSO-d₆) δ 10.93 (s, 1H), 9.58 (s, 1H), 9.42 (s, 1H), 8.85 (s, 1H), 8.40-8.30 (m, 1H), 7.89 (d, J=8.8 Hz, 1H), 7.76-7.66 (m, 1H), 7.46 (d, J=7.2 Hz, 1H), 3.07 (d, J=4.4 Hz, 3H), 2.10-2.00 (m, 1H), 0.90-0.80 (m, 4H).

### Example 491: Synthesis of N-(5-(8-chloro-[1,2,4]triazolo[1,5-a]pyridin-2-yl)-8-(methylamino)-2,7-naphthyridin-3-yl)cyclopropanecarboxamide

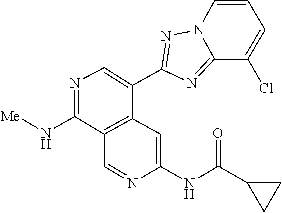 [SMILES: CNc1ncc(-c2nc3c(Cl)cccn3n2)c2cc(NC(=O)C3CC3)ncc12]

### Step 1: 2-bromo-8-chloro[1,2,4]triazolo[1,5-a]pyridine

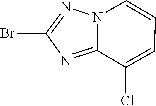 [SMILES: Clc1cccn2nc(Br)nc12]

A solution of tert-butyl nitrite (244.6 mg; 2.372 mmol; 2.00 eq.) and CuBr (340.4 mg; 2.372 mmol; 2.00 eq.) in MeCN (5 mL) was stirred at 60° C. for 10 minutes. To the above solution was added a solution of 8-chloro-[1,2,4]triazolo[1,5-a]pyridin-2-amine (200.0 mg; 1.186 mmol; 1.00 eq.) in MeCN (5 mL) dropwise at room temperature. The resulting mixture was stirred at 60° C. for 1 hour. The desired product was detected via LCMS. The mixture was allowed to cool down to room temperature. The mixture was acidified to pH=4 with a 1 M HCl solution. The resulting mixture was extracted with EtOAc (2×25 mL), The combined organic layers were washed with brine (3×5 mL), dried over anhydrous Na₇SO₄, filtered, and concentrated under reduced to pressure to afford 2-bromo-8-chloro-[1,2,4]triazolo[1-5-a]pyridine as a yellow solid (178.0 mg, 64.7%). LCMS (ESI) m/z 231.9, [M+H]⁺.

### Step 2: N-(5-(8-chloro-[1,2,4]triazolo[1,5-a]pyridin-2-yl)-8-(methylamino)-2,7-naphthyridin-3-yl)cyclopropanecarboxamide

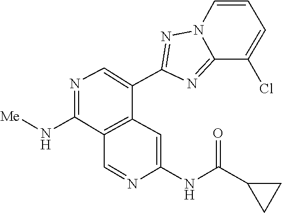 [SMILES: CNc1ncc(-c2nc3c(Cl)cccn3n2)c2cc(NC(=O)C3CC3)ncc12]

N-(5-(8-chloro-[1,2,4]triazolo[1,5-a]pyridin-2-yl)-8-(methylamino)-2,7-naplathyridin-3-yl)cyclopropanecarboxamide was synthesized using a similar procedure that was previously described in Example 470, by using N-(8-(methylamino)-5-(4,4,5,5-tetramethyl-1,3,2-dioxaborolan-2-yl)-2,7-naphthyridin-3-yl)cyclopropanecarboxamide and 2-bromo-8-chloro-[1,2,4]triazolo[1,5-a]pyridine as the starting material. LCMS (ESI) m/z 394.1, [M+H]⁺. ¹H NMR (400 MHz, DMSO-d₆) δ 10.95 (s, 1H), 9.50 (s, 1H), 9.42 (s, 1H), 8.95 (d, J=6.8 Hz, 1H), 8.85 (s, 1H), 8.40-8.32 (m, 1H), 7.88 (d, J=7.6 Hz, 1H), 7.23-7.15 (m, 1H), 3.07 (d, J=4.4 Hz, 3H), 2.12-2.03 (m, 1H), 0.91-0.79 (m, 4H).

### Example 492

### Synthesis of N-(5-(7-methoxy-[1,2,4]triazolo[1,5-a]pyridin-2-yl)-8-(methylamino)-2,7-naphthyridin-3-yl)cyclopropanecarboxamide

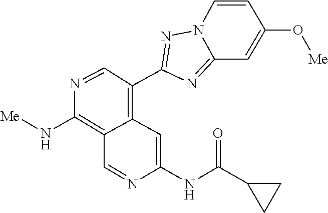 [SMILES: CNc1ncc(-c2nc3cc(OC)ccn3n2)c2cc(NC(=O)C3CC3)ncc12]

### Step 1: 7-bromo-2-chloro-[1,2,4]triazolo[1,5-a]pyridine

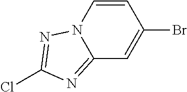 [SMILES: Clc1nc2cc(Br)ccn2n1]

To a stirring mixture of 7-bromo-[1,2,4]triazolo[1,5-a]pyridin-2-amine (1.00 g; 4.694 mmol; 1.00 eq.) and CuCl₂ (378.6 mg; 2.81 mmol; 0.60 eq.) in HCl (12 M in water, 15 mL) was added a solution of NaNO₂ (390.1 mg; 5.654 mmol; 1.20 eq.) in water (3 mL) dropwise at 0° C. The reaction mixture was stirred at room temperature overnight. The desired product was detected via LCMS. The resulting mixture was added dropwise to water (100 mL) and precipitates were formed. The precipitated solids were collected by filtration and washed with water (3×20 mL) to provide 7-bromo-2-chloro-[1,2,4]triazolo[1,5-a]pyridine as a yellow solid (950.0 mg, 87.0%). LCMS (ESI) m/z 231.9, [M+H]⁺.

### Step 2: 2-chloro-7-methoxy-[1,2,4]triazolo[1,5-a]pyridine

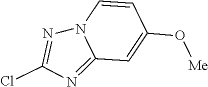 [SMILES: COc1ccn2nc(Cl)nc2c1]

To a stirring mixture of 7-bromo-2-chloro-[1,2,4]triazolo[1,5-a]pyridine (200.0 mg; 0.860 mmol; 1.00 eq.), Pd₂(dba)₃ (94.5 mg; 0.103 mmol; 0.12 eq.), t-BuXPhos (87.9 mg; 0.207 mmol; 0.24 eq.) and Cs₂CO₃ (840.9 mg; 2.581 mmol; 3.00 eq.) in toluene (8 mL) was added MeOH (27.3 mg; 0.852 mmol; 0.99 eq.) under nitrogen atmosphere. The mixture was stirred at 90° C. for 2 hours. The desired product was detected via LCMS. The solvent was concentrated under vacuum. The residue was purified by flash chromatography on silica gel column using 0-10% of MeOH in CH₂Cl₂ as eluent to provide 2-chloro-7-methoxy-[1,2,4]triazolo[1,5-a]pyridine as a yellow solid (138.2 mg, 87.1%). LCMS (ESI) m/z 184.0, [M+H]⁺.

### Step 3: N-(5-(7-methoxy-[1,2,4]triazolo[1,5-a]pyridin-2-yl)-8-(methylamino)-2,7-naphthyridin-3-yl)cyclopropanecarboxamide

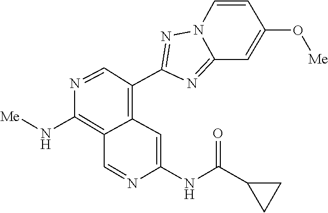 [SMILES: CNc1ncc(-c2nc3cc(OC)ccn3n2)c2cc(NC(=O)C3CC3)ncc12]

N-(5-(7-methoxy-[1,2,4]triazolo[1,5-a]pyridin-2-yl)-8-(methylamino)-2,7-naphthyridin-3-yl)cyclopropanecarboxamide was synthesized using a similar procedure that was previously described in Example 470 by using N-(8-(methylamino)-5-(4,4,5,5-tetramethyl-1,3,2-dioxaborolan-2-yl)-2,7-naphthyridin-3-yl)cyclopropanecarboxamide and 2-chloro-7-methoxy-[1,2,4]triazolo[1,5-a]pyridine as the starting material. LCMS (ESI) m/z 390.2, ¹H NMR (400 MHz, DMSO-d₆) δ 10.92 (s, 1H), 9.47 (s, 1H), 9.40 (s, 1H), 8.78 (s, 1H), 8.75 (d, J=7.6 Hz, 1H), 8.28-8.23 (m, 1H), 7.24 (d, J=2.4 Hz, 1H), 6.82 (dd, J=7.6, 2.4 Hz, 1H), 3.93 (s, 3H), 3.05 (d, J=4.4 Hz, 3H), 2.1.1-2.04 (m, 1H), 0.88-0.80 (m, 4H).

### Example 493: Synthesis of N-(5-(6-(1,1-difluoroethyl)-[1,2,4]triazolo[1,5-a]pyridin-2-yl)-8-(methylamino)-2,7-naphthyridin-3-yl)cyclopropanecarboxamide

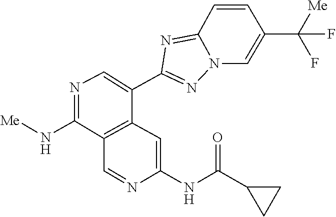 [SMILES: CNc1ncc(-c2nc3ccc(C(C)(F)F)cn3n2)c2cc(NC(=O)C3CC3)ncc12]

### Step 1: 2-chloro-6-(1-ethoxyvinyl)-[1,2,4]triazolo[1,5-a]pyridine

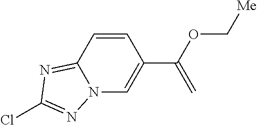

A mixture of 6-bromo-2-chloro-[1,2,4]triazolo[1,5-a]pyridine (200.0 mg; 0.860 mmol; 1.00 eq.), tributyl(1-ethoxyethenyl)stannane (373.0 mg; 1.033 mmol; 1.20 eq.) and Pd(PPh₃)₂Cl₂ (30.0 mg; 0.043 mmol; 0.05 eq.) in DMF (5 mL) was stirred at 60° C. for 2 hours under nitrogen atmosphere. The desired product was detected via LCMS. The mixture was diluted with water (30 mL) and extracted with EtOAc (3×30 mL). The combined organic layers were washed with brine (2×20 mL), dried over anhydrous Na₂SO₄. The organic layer was concentrated under vacuum. The residue was purified by flash chromatography on silica gel column using 10-30% of EtOAc in petroleum ether as eluent to provide 2-chloro-6-(1-ethoxyvinyl)-[1,2,4]triazolo[1,5-a]pyridine as a white solid (188.0 mg, 97.7%). LCMS (ESI) m/z 224.1, [M+H]⁺.

### Step 2: 1-(2-chloro-[1,2,4]triazolo[1,5-a]pyridin-6-yl)ethan-1-one

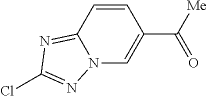 [SMILES: CC(=O)c1ccc2nc(Cl)nn2c1]

To a solution of 2-chloro-6-(1-ethoxyvinyl)-[1,2,4]triazolo[1,5-a]pyridine (400.0 mg; 1.788 mmol; 1.00 eq.) in DMF (5 mL) was added HCl (3 M in water, 2.5 mL) at 0° C. The resulting mixture was stirred at room temperature for 3 hours. The desired product was detected via LCMS. The mixture was purified by flash chromatography on pre-packed C18 column using 50-80% of MeOH in water (10 mmol/L NH₄HCO₃) as eluent to provide 1-(2-chloro-[1,2,4]triazolo[1,5-a]pyridin-6-yl)ethan-1-one as a white solid (253.0 mg, 72.3%). LCMS (ESI) m/z 196.0, [M+H]⁴.

### Step 3: 2-chloro-6-(1,1-difluoroethyl)-[1,2,4]triazolo[1,5-a]pyridine

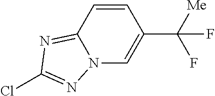 [SMILES: CC(F)(F)c1ccc2nc(Cl)nn2c1]

A solution of 1-(2-chloro-[1,2,4]triazolo[1,5-a]pyridin-6-yl)ethan-1-one (224.0 mg; 1.145 mmol; 1.00 eq.) in diethylaminosulfur trifluoride (5 mL) was stirred at room temperature overnight under nitrogen atmosphere. The desired product was detected via LCMS. The mixture was quenched with a saturated NaHCO₃ aqueous solution (30 mL) and extracted with EtOAc (3×30 mL). The combined organic layers were washed with brine (2×10 mL), dried over anhydrous Na₂SO₄, and concentrated under reduced pressure. The residue was purified by flash chromatography on pre-packed C18 column using 30-50% of MeCN in water (10 mmol/L NH₄HCO₃) as eluent to provide 2-chloro-6-(1,1-difluoroethyl)-[1,2,4]triazolo[1,5-a]pyridine as a yellow solid (200.0 mg, 80.3%). LCMS (ESI) rah 218.0, [M+H]⁺.

### Step 4: N-(5-(6-(1,1-difluoroethyl)-[1,2,4]triazolo[1,5-a]pyridin-2-yl)-8-(methylamino)-2,7-naphthyridin-3-yl)cyclopropanecarboxamide

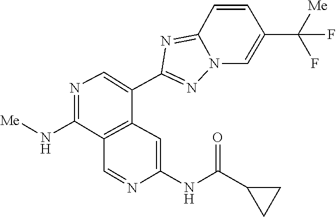 [SMILES: CNc1ncc(-c2nc3ccc(C(C)(F)F)cn3n2)c2cc(NC(=O)C3CC3)ncc12]

N-(5-(6-(1,1-difluoroethyl)-[1,2,4]triazolo[1,5-a]pyridin-2-yl)-8-(methylamino)-2,7-naphthyridin-3-yl)cyclopropanecarboxamide was synthesized using a similar procedure that was previously described in Example 470 by using N-(8-(methylamino)-5-(4,4,5,5-tetramethyl-1,3,2-dioxaborolan-2-yl)-2,7-naphthyridin-3-yl)cyclopropanecarboxamide and 2-chloro-6-(1,1-difluoroethyl)-[1,2,4]triazolo-[1,5-a]pyridine as the starting material. LCMS (ESI) m/z 424.2, ¹H NMR (400 MHz, DMSO-d₆) δ 10.97 (s, 1H), 9.49 (s, 1H), 9.42 (s, 1H), 9.23 (d, J=2.0 Hz, 1H), 8.84 (s, 1H), 8.40-8.29 (m, 1H), 7.98 (d, J=9.6 Hz, 1H), 7.86 (dcl, J=9.6, 2.0 Hz, 3.07 (d, J=4.4 Hz, 3H), 2.14 (t, J=18.8 Hz, 3H), 2.10-2.04 (m, 1H), 0.91-0.79 (m, 4H).

### Example 493A: Synthesis of N-(8-(methylamino)-5-(6-(trifluoromethoxy)-[1,2,4]triazolo[1,5-a]pyridin-2-yl)-2,7-naplathyridin-3-yl)cyclopropanecarboxamide

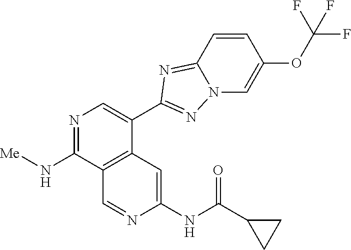 [SMILES: CNc1ncc(-c2nc3ccc(OC(F)(F)F)cn3n2)c2cc(NC(=O)C3CC3)ncc12]

### Step 1: N-(4-methoxybenzyl)-5-(trifluoromethoxy)pyridin-2-amine

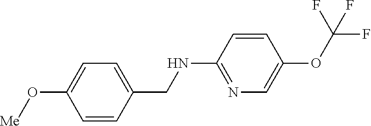 [SMILES: COc1ccc(CNc2ccc(OC(F)(F)F)cn2)cc1]

To a solution of (4-methoxyphenyl)methanamine (694.4 mg; 5.062 mmol; 2.00 eq.) in NMP (3 mL) were added 2-chloro-5-(trifluoromethoxy)pyridine (500.0 mg; 2.531 mmol; 1.00 eq.) and DIPEA (981.4 mg; 7.593 mmol; 3.00 eq.) at room temperature. The resulting mixture was stirred at 140° C. overnight under nitrogen atmosphere. The desired product was detected via LCMS. The mixture was allowed to cool down to room temperature. The resulting mixture was diluted with water (20 mL) and extracted with EtOAc (3×30 mL). The combined organic layers were washed with brine (3×10 mL), dried over anhydrous Na₂SO₄, filtered, and concentrated under reduced pressure. The residue was purified by flash chromatography on silica gel column using 5-8% of MeOH in CH₂Cl₂ as eluent to provide N-(4-methoxybenzyl)-5-(trifluoromethoxy)pyridin-2-amine as a yellow solid (300.0 mg, 39.1%). LCMS (ESI) m/z 299.1, [M+H]⁺.

### Step 2: 5-(trifluoromethoxy)pyridin-2-amine

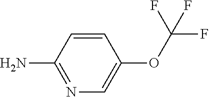 [SMILES: Nc1ccc(OC(F)(F)F)cn1]

A solution of N-(4-methoxybenzyl)-5-(trifluoromethoxy)pyridin-2-amine (300.0 mg; 1.006 mmol; 1.00 eq.) in TFA (3 mL) was stirred at 60° C. for 1 hour under nitrogen atmosphere. The desired product was detected via LCMS. The mixture was allowed to cool down to room temperature. The resulting mixture was diluted with water (20 mL) and neutralized to pH=7 with a saturated NaHCO₃ aqueous solution. The resulting mixture was extracted with EtOAc (3×20 mL). The combined organic layers were washed with brine (3×10 mL), dried over anhydrous Na₂SO₄, filtered, and concentrated under reduced pressure. The residue was purified by flash chromatography on silica gel column using 20-30% of CH₂Cl₂ in petroleum ether as eluent to provide 5-(trifluoromethoxy)pyridin-2-amine as a light yellow solid (150.0 mg, 83.7%). LCMS (ESI) m/z 179.0, [M+H]⁺.

### Step 3: 6-(trifluoromethoxy)-[1,2,4]triazolo[1,5-a]pyridin-2-amine

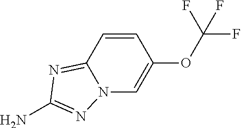 [SMILES: Nc1nc2ccc(OC(F)(F)F)cn2n1]

A mixture of 5-(trifluoromethoxy)pyridin-2-amine (200.0 mg; 1.123 mmol; 1.00 eq.) and O-ethyl carbonisothiocyanatidate (162.0 mg; 1.235 mmol; 1.10 eq.) in 1,4-dioxane (2 mL) was stirred at room temperature overnight under nitrogen atmosphere. The desired product was detected via LCMS. The resulting mixture was concentrated under reduced pressure to afford the intermediate. The intermediate was dissolved in a mixture solvent of MeOH/EtOH (1:1, 2 mL). To this crude mixture were added hydroxylamine hydrochloride (117.0 mg; 1.684 mmol; 1.50 eq.) and DIPEA (217.7 mg; 1.684 mmol; 1.50 eq.). The resulting mixture was stirred at 60° C. for 2 hours under nitrogen atmosphere. Upon completion, the mixture was allowed to cool down to room temperature. The resulting mixture was concentrated under reduced pressure. The residue was purified by flash chromatography on silica gel column using 10-30% of EtOAc in petroleum ether as eluent to provide 6-(trifluoromethoxy)-[1,2,4]triazolo[1,5-a]pyridin-2-amine as a light yellow solid (150.0 mg, 61.2%). LCMS (ESI) m/z 219.0, [M+H]⁺.

### Step 4: 2-chloro-6-(trifluoromethoxy)-[1,2,4]triazolo[1,5-a]pyridine

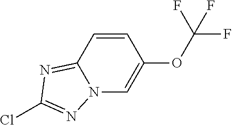 [SMILES: FC(F)(F)Oc1ccc2nc(Cl)nn2c1]

To a stirred solution of 6-(trifluoromethoxy)-[1,2,4]triazolo[1,5-a]pyridin-2-amine (150.0 mg; 0.688 mmol; 1.00 eq.) in HCl (12 M in water, 4 mL) was added CuCl₂ (27.7 mg; 0.206 mmol; 0.30 eq.) and a solution of NaNO₂ (71.2 mg; 1.032 mmol; 1.50 eq.) in water (1 mL) at 0° C. The resulting mixture was stirred at room temperature for 1 hour. The desired product was detected via LCMS. The resulting mixture was diluted with water (20 mL). The mixture was neutralized to pH=7 with a saturated NaHCO₃ solution and extracted with EtOAc (3×50 mL). The combined organic layers were washed with brine (3×10 mL) and dried over anhydrous Na₂SO₄. The organic layer was concentrated under reduced pressure to afford 2-chloro-6-(trifluoromethoxy)-[1,2,4]triazolo[1,5-a]pyridine as a off-white solid (120.0 mg, 73.5%). LCMS (ESI) m/z 238.0, [M+H]⁺.

### Step 5: N-(8-(methylamino)-5-(6-(trifluoromethoxy)-[1,2,4]triazolo[1,5-a]pyridin-2-yl)-2,7-naphthyridin-3-yl)cyclopropanecarboxamide

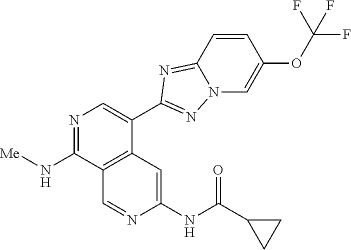 [SMILES: CNc1ncc(-c2nc3ccc(OC(F)(F)F)cn3n2)c2cc(NC(=O)C3CC3)ncc12]

N-(8-(methylamino)-5-(6-(trifluoromethoxy)-[1,2,4]triazolo[1,5-a]pyridin-2-yl)-2,7-naphthyridin-3-yl)cyclopropanecarboxamide was synthesized using a similar procedure that was previously described in Example 470 by using N-(8-(methylamino)-5-(4,4,5,5-tetramethyl-1, 3,2-dioxaborolan-2-yl)-2,7-naphthyridin-3-yl)cyclopropanecarboxamide and 2-chloro-6-(trifluoromethoxy)-[1,2,4]triazolo[1,5-a]pyridine as the starting material. LCMS (ESI) m/z 444.1, [M+H]⁺. ¹H NMR (300 MHz, DMSO-d₆) δ 1.0.94 (s, 1H), 9.52-9.47 (m, 1H), 9.45 (s, 1H), 9.42 (s, 1H), 8.81 (s, 1H), 8.38-8.31 (m, 1H), 8.03-7.96 (m, 1H), 7.89-7.79 (m, 1H), 3.07 (d, J=4.4 Hz, 3H), 2.1.5-1.99 (m, 1H), 0.90-0.80 (m, 4H).

### Example 494: Synthesis of N-(5-(6-cyclopropoxy-[1,2,4]triazolo[1,5-a]pyridin-2-yl)-8-(methylamino)-2,7-naphthyridin-3-yl)cyclopropanecarboxamide

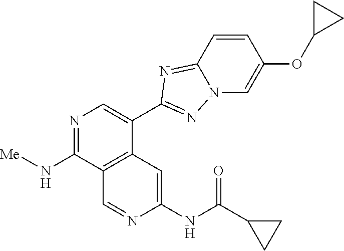 [SMILES: CNc1ncc(-c2nc3ccc(OC4CC4)cn3n2)c2cc(NC(=O)C3CC3)ncc12]

### Step 1: 5-cyclopropoxy-2-nitropyridine

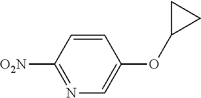 [SMILES: O=[N+]([O-])c1ccc(OC2CC2)cn1]

To a stirred solution of cyclopropanol (740.1 mg; 12.740 mmol; 1.00 eq.) in THF (20 mL) was added NaH (60%) (1.16 g; 29.004 mmol; 2.1.0 eq.) in portions at 0° C. The mixture was stirred at room temperature for 0.5 hour under nitrogen atmosphere. To the above solution was added 5-fluoro-2-nitropyridine (2.01 g; 14.071 mmol; 1.10 eq.) at 0° C. The resulting mixture was stirred at room temperature for 1 hour. The desired product was detected via LCMS. The reaction was quenched with saturated. NH₄Cl solution (50 mL) and extracted with EtOAc (3×20 mL). The combined organic layers were washed with brine (3×10 mL) and dried over anhydrous Na₂SO₄, filtered, and concentrated under reduced pressure. The residue was purified by flash chromatography on silica gel column using 20-30% of EtOAc in petroleum ether as eluent to provide 5-cyclopropoxy-2-nitropyridine as a yellow solid (1.50 g, 59.1%). LCMS (ESI) m/z 181.1, [M+H]⁺.

### Step 2: 5-cyclo propoxypyridin-2-amine

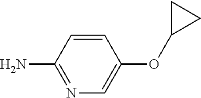 [SMILES: Nc1ccc(OC2CC2)cn1]

To a stirring mixture of 5-cyclopropoxy-2-nitropyridine (560.1 mg; 3.101 mmol; 1.00 eq.) in MeOH (6 mL) was added 10% Pd/C (170.2 mg; 30% w/w) under nitrogen atmosphere. The mixture was hydrogenated at 40° C. for 1 hour under hydrogen atmosphere (2 atm). The desired product was detected via LCMS. The resulting mixture was filtered, the filter cake was washed with MeOH (5 mL). The filtrate was concentrated under reduced pressure to afford 5-cyclopropoxypyridin-2-amine as a black oil (470.0 mg, 98.6%). LCMS (ESI) m/z 151.1, [M+H]⁺.

### Step 3: ethyl N-[(5-cyclopropoxypyridin-2-yl)carbamothioyl]carbamate

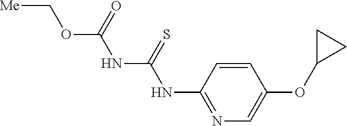 [SMILES: CCOC(=O)NC(=S)Nc1ccc(OC2CC2)cn1]

A solution of 5-cyclopropoxypyridin-2-amine (460.1 mg; 3.060 mmol; 1.00 eq.) and O-ethyl carbonisothiocyanatidate (442.1 mg; 3.372 mmol; 1.10 eq.) in 1,4-dioxane (5 mL) was stirred at room temperature overnight under nitrogen atmosphere. The desired product was detected via LCMS. The resulting mixture was concentrated under reduced pressure to afford ethyl N-[(5-cyclopropoxypyridin-2-yl)carbamothioyl]carbamate as a yellow solid (800.1 mg, crude). LCMS (ESI) m/z 282.1, [M+H]⁺.

### Step 4: 6-cyclopropoxy-[1,2,4]triazolo[1,5-a]pyridin-2-amine

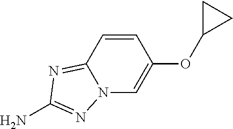 [SMILES: Nc1nc2ccc(OC3CC3)cn2n1]

To solution of ethyl N-[(5-cyclopropoxypyridin-2-yl)carbamothioyl]carbamate (800.1 mg; 2.952 mmol; 1.00 eq.) and hydroxylamine hydrochloride (306.0 mg; 4.401 mmol; 1.50 eq.) in a mixture solvent of MeOH/EtOH (1:1, 5 mL) was added DIPEA (571.1 mg; 4.411 mmol; 1.00 eq.). The mixture was stirred at 60° C. for 4 hours under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide 6-cyclopropoxy-[1,2,4]triazolo[1,5-a]pyridin-2-amine as a white solid (190.1 mg, 33.8%). LCMS (ESI) m/z 191.1, [M+H]⁺.

### Step 5: 2-bromo-6-cyclopropoxy-[1,2,4]triazolo[1,5-a]pyridine

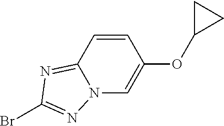 [SMILES: Brc1nc2ccc(OC3CC3)cn2n1]

To a stirring mixture of 6-cyclopropoxy-[1,2,4]triazolo[1,5-a]pyridin-2-amine (150.3 mg; 0.782 mmol; 0.60 eq.) and CuBr (168.2 mg; 1.172 mmol; 1.00 eq.) in MeCN (3 mL) was added tert-butyl nitrite (121.1 mg; 1.171 mmol; 1.00 eq.). The resulting mixture was stirred at 60° C. for 1 hour. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide 2-bromo-6-cyclopropoxy-[1,2,4]triazolo[1,5-a]pyridine as a white solid (70.2 mg, 34.9%). LCMS (ESI) m/z 254.0, [M+H]⁺.

### Step 6: N-(5-(6-cyclopropoxy-[1,2,4]triazolo[1,5-a]pyridin-2-yl)-8-(methylamino)-2,7-naphthyridin-3-yl)cyclopropanecarboxamide

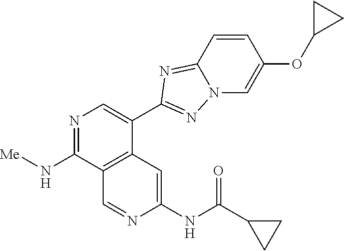 [SMILES: CNc1ncc(-c2nc3ccc(OC4CC4)cn3n2)c2cc(NC(=O)C3CC3)ncc12]

N-(5-(6-cyclopropoxy-[1,2,4]triazolo[1,5-a]pyridin-2-yl)-8-(methylamino)-2,7-naphthyridin-3-yl)cyclopropanecarboxamide was synthesized using a similar procedure that was previously described in Example 470, by using N-(8-(methylamino)-5-(4,4,5,5-tetramethyl-1,3,2-dioxaborolan-2-yl)-2,7-naphthyridin-3-yl)cyclopropanecarboxamide and 2-bromo-6-cyclopropoxy-[1,2,4]triazolo[1,5-a]pyridine as the starting material. LCMS (ESI) m/z 416.2, [M+H]⁺. ¹H NMR (400 MHz, DMSO-d₆) δ 10.98 (s, 1H), 9.42 (s, 1H), 9.41 (s, 1H), 8.78 (d, J=2.0 Hz, 1H), 8.69 (s, 1H), 8.48-8.28 (m, 1H), 7.79 (d, J=9.6 Hz, 1H), 7.47 (d, J=9.6, 2.0 Hz, 1H), 4.1.2-4.02 (m, 1H), 3.07 (d, J=4.4 Hz, 3H), 2.12-2.02 (m, 1H), 0.97-0.63 (m, 8H).

### Example 495: Synthesis of N-(5-(6-(cyclopropylmethoxy)-[1,2,4]triazolo[1,5-a]pyridin-2-yl)-8-(methylamino)-2,7-naphthyridin-3-yl)cyclopropanecarboxamide

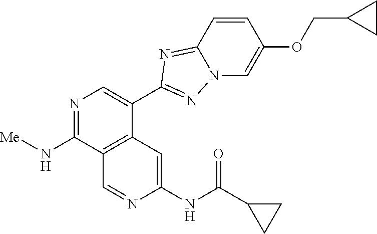 [SMILES: CNc1ncc(-c2nc3ccc(OCC4CC4)cn3n2)c2cc(NC(=O)C3CC3)ncc12]

### Step 1: 5-(cyclopropylmethoxy)-2-nitropyridine

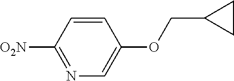 [SMILES: O=[N+]([O-])c1ccc(OCC2CC2)cn1]

To a stirred solution of cyclopropylmethanol (0.51 g; 7.038 mmol; 1.00 eq.) in THF (10 mL) was added NaH: (60%) (0.51 g; 21.1.14 mmol; 3.00 eq.) at 0° C. under nitrogen atmosphere. After stirring at room temperature for 0.5 hour, to the solution was added 5-fluoro-2-nitropyridine (1.00 g; 7.038 mmol; 1.00 eq.) at 0° C. The resulting mixture was stirred at room temperature for 2 hours. The desired product was detected via LCMS. The reaction was quenched with a saturated NH₄Cl aqueous solution (10 mL) and extracted with EtOAc (3×10 mL). The combined organic layers were washed with water (3×10 mL) and dried over anhydrous Na₂SO₄, filtrated, and concentrated under reduced pressure. The residue was purified by flash chromatography on silica gel column using 10-30% of EtOAc in petroleum ether as eluent to provide 5-(cyclopropylmethoxy)-2-nitropyridine as an off-white solid (1.00 g, 73.2%). LCMS (ESI) m/z 195.1, [M+H]⁺.

### Step 2: 5-(cyclopropylmethoxy)pyridin-2-amine

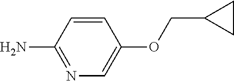 [SMILES: Nc1ccc(OCC2CC2)cn1]

To a solution of 5-(cyclopropylmethoxy)-2-nitropyridine (500.0 mg; 2.575 mmol; 1.00 eq.) in MeOH (1.0 mL) was added 10% Pd/C (500.0 mg; 1.00% w/w) under nitrogen atmosphere. The mixture was hydrogenated at room temperature for 3 hours under hydrogen atmosphere (2 atm). After filtration, the filtrate was concentrated under reduced pressure to afford 5-(cyclopropylmethoxy)pyridin-2-amine as a white solid (420.0 mg, crude). LCMS (ESI) m/z 165.1, [M+H]⁺.

### Step 3: ethyl N-{[5-(cyclopropylmethoxy)pyridin-2-yl]carbamothioyl}carbamate

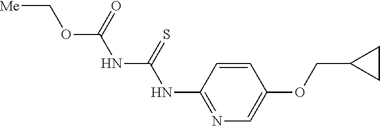 [SMILES: CCOC(=O)NC(=S)Nc1ccc(OCC2CC2)cn1]

A solution of 5-(cyclopropylmethoxy)pyridin-2-amine (450.0 mg; 2.740 mmol; 1.00 eq.) and O-ethyl carbonisothiocyanatidate (395.4 mg; 3.014 mmol; 1.10 eq.) in 1,4-dioxane (5 mL) was stirred at room temperature overnight under nitrogen atmosphere. The desired product was detected via LCMS. The resulting mixture was concentrated under reduced pressure. The residue was purified by flash chromatography on silica gel column using 10-30% of CH₂Cl₂ in petroleum ether as eluent to provide ethyl N-{[5-(cyclopropylmethoxy)pyridin-2-yl]carbamothioyl}carbamate as a brown solid (600.0 mg, 74.1%). LCMS (ESI) m/z 296.1, [M+H]⁺.

### Step 4: 6-(cyclopropylmethoxy)-[1,2,4]triazolo[1,5-a]pyridin-2-amine

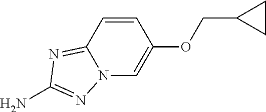 [SMILES: Nc1nc2ccc(OCC3CC3)cn2n1]

To a solution of ethyl N-{[5-(cyclopropylmethoxy)pyridin-2-yl]carbamothioyl}carbamate (500.0 mg; 1.693 mmol; 1.00 eq.) and hydroxylamine hydrochloride (176.5 mg; 2.540 mmol; 1.50 eq.) in a mixture solvent of MeOH/EtOH (1:1, 12 mL) was added DIPEA (328.2 mg; 2.540 mmol; 1.50 eq.) at room temperature under nitrogen atmosphere. The resulting mixture was stirred at 60° C. for 2 hours. The desired product was detected via LCMS. The mixture was allowed to cool down to room temperature. The resulting mixture was concentrated under reduced pressure. The residue was purified by flash chromatography on silica gel column using 1.0-30% of EtOAc in petroleum ether as eluent to provide 6-(cyclopropylmethoxy)-[1,2,4]triazolo[1,5-a]pyridin-2-amine as a white solid (170.0 mg, 49.2%). LCMS (ESI) m/z 205.1, [M+H]⁺.

### Step 5: 2-chloro-6-(cyclopropylmethoxy)-[1,2,4]triazolo[1,5-a]pyridine

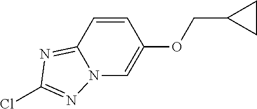 [SMILES: Clc1nc2ccc(OCC3CC3)cn2n1]

To a solution of 6-(cyclopropylmethoxy)-[1,2,4]triazolo[1,5-a]pyridin-2-amine (170.0 mg; 0.832 mmol; 1.00 eq.) in HCl (12 M in water, 2 mL) was added CuCl₂ (33.6 mg; 0.250 mmol; 0.30 eq.), and a solution of NaNO₂ (86.2 mg; 1.248 mmol; 1.50 eq.) in water (0.5 mL) at 0° C. The resulting mixture was stirred at room temperature for 1 hour. The desired product was detected via LCMS. The resulting mixture was diluted with water (20 mL) and neutralized to pH=7 with a saturated NaHCO₃ aqueous solution. The resulting mixture was extracted with EtOAc (3×30 mL). The combined organic layers were washed with brine (3×10 mL), dried over anhydrous Na₂SO₄, filtered, and concentrated under reduced pressure to afford 2-chloro-6-(cyclopropylmethoxy)-[1,2,4]triazolo[1,5-a]pyridine as an off-white solid (60.0 mg, 32.2%). LCMS (ESI) m/z 224.1, [M+H]⁺.

### Step 6: N-(5-(6-(cyclopropylmethoxy)-[1,2,4]triazolo[1,5-a]pyridin-2-yl)-8-(methylamino)-2,7-naphthyridin-3-yl)cyclopropanecarboxamide

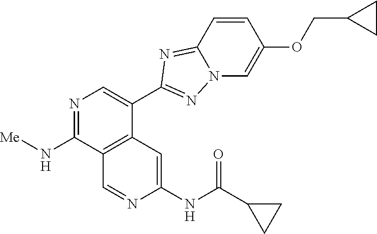 [SMILES: CNc1ncc(-c2nc3ccc(OCC4CC4)cn3n2)c2cc(NC(=O)C3CC3)ncc12]

N-(5-(6-(cyclopropylmethoxy)-[1,2,4]triazolo[1,5-a]pyridin-2-yl)-8-(methylamino)-2,7-naphthyridin-3-yl)cyclopropanecarboxamide was synthesized using a similar procedure that was previously described in Example 470 by using N-(8-(methylamino)-5-(4,4,5,5-tetramethyl-1,3,2-dioxaborolan-2-yl)-2,7-naphthyridin-3-yl)cyclopropanecarboxamide and 2-chloro-6-(cyclopropylmethoxy)-[1,2,4]triazolo[1,5-a]pyridine as the starting material. LCMS (ESI) m/z 430.2, [M+H]⁺. ¹H NMR (300: MHz, DMSO-d₆) δ 1.0.90 (s, 1H), 9.40 (s, 1H), 9.39 (s, 1H), 8.72 (s, 1H), 8.61 (d, J=2.0 Hz, 1H), 8.28-8.20 (m, 1H), 7.77 (d, J=9.6 Hz, 1H), 7.48 (dd, J=9.6, 2.0 Hz, 1H), 3.95 (d, J=9.6 HZ, 2H),3.05 (d, J=4.4 Hz, 3H), 2.14-2.00 (m, 1H), 1.36-1.21 (m, 1H), 0.92-0.76 (m, 4H), 0.68-0.54 (m, 2H), 0.44-0.33 (m, 2H).

### Example 496: Synthesis of N-(8-(methylamino)-5-(6-(trifluoromethyl)-[1,2,4]triazolo[1,5-a]pyridin-2-yl)-2,7-naphthyridin-3-yl)cyclopropanecarboxamide

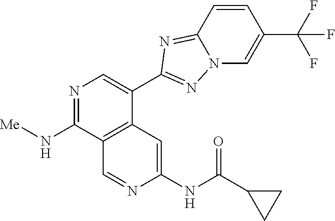 [SMILES: CNc1ncc(-c2nc3ccc(C(F)(F)F)cn3n2)c2cc(NC(=O)C3CC3)ncc12]

### Step 1: N-{[5-(trifluoromethyl)pyridin-2-yl]carbamothioyl}carbamate

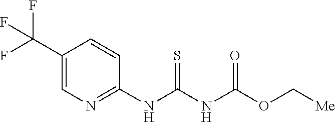 [SMILES: CCOC(=O)NC(=S)Nc1ccc(C(F)(F)F)cn1]

A mixture of 5-(trifluoromethyl)pyridin-2-amine (1.00 g; 6.168 mmol; 1.00 eq.) and O-ethyl carbonisothiocyanatidate (0.89 g; 6.785 mmol; 1.10 eq.) in 1,4-dioxane (10 mL) was stirred at room temperature overnight under nitrogen atmosphere. The desired product was detected via LCMS. The resulting mixture was concentrated under reduced pressure. The residue was purified by flash chromatography on silica gel column using 10-30% of CH₂Cl₂ in petroleum ether as eluent to provide ethyl N-{[5-(trifluoromethyl)pyridin-2-yl]carbamothioyl}carbamate as a brown solid (1.30 g, 71.9%). LCMS (ESI) m/z 294.0, [M+H]⁺.

### Step 2: 6-(trifluoromethyl)-[1,2,4]triazolo[1,5-a]pyridin-2-amine

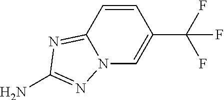 [SMILES: Nc1nc2ccc(C(F)(F)F)cn2n1]

To a solution of ethyl N-{[5-(trifluoromethyl)pyridin-2-yl]carbamothioyl}carbamate (1.50 g; 5.12 mmol; 1.00 eq.) and hydroxylamine hydrochloride (0.53 g; 7.67 mmol; 1.50 eq.) in a mixture solvent of MeOH/EtOH (1:1, 15 mL) was added DIPEA (0.99 g; 7.67 mmol; 1.50 eq.) at room temperature. The resulting mixture was stirred at 60° C. for 2 hours under nitrogen atmosphere. The desired product was detected via LCMS. The mixture was allowed to cool down to room temperature. The resulting mixture was concentrated under reduced pressure. The residue was purified by flash chromatography on silica gel column using 1.0-30% of EtOAc in petroleum ether as eluent to provide 6-(trifluoromethyl)-[1,2,4]triazolo[1,5-a]pyridin-2-amine as an off-white solid (700.0 mg, 67.7%). LCMS (ESI) m/z 203.0, [M+H]⁺.

### Step 3: 2-chloro-6-(trifluoromethyl)-[1,2,4]triazolo[1,5-a]pyridine

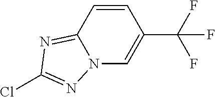 [SMILES: FC(F)(F)c1ccc2nc(Cl)nn2c1]

To a stirring solution of 6-(trifluoromethyl)-[1,2,4]triazolo[1,5-a]pyridin-2-amine (700.0 mg; 3.463 mmol; 1.00 eq.) in MI (12 M in water, 8 mL) was added CuCl₂ (1.397 mg; 1.039 mmol; 0.30 eq.), and a solution of NaNO₂ (358.4 mg; 5.194 mmol; 1.50 eq.) in water (2 mL) at 0° C. The mixture was stirred at room temperature for 1 hour under nitrogen atmosphere. The desired product was detected via LCMS. The resulting mixture was diluted with water (20 mL) and neutralized to pH=7 with a saturated NaHCO₃ aqueous solution. The resulting mixture was extracted with EtOAc (3×50 mL). The combined organic layers were washed with water (3×20 mL), dried over anhydrous Na₂SO₄, filtered, and concentrated under reduced pressure to afford 2-chloro-6-(trifluoromethyl)-[1,2,4]triazolo[1,5-a]pyridine as a light yellow solid (450.0 mg, 58.6%). LCMS (ESI) m/z 222.0, [M+H]⁺.

### Step 4: N-(8-(methylamino)-5-(6-(trifluoromethyl)-[1,2,4]triazolo[1,5-a]pyridin-2-yl)-2,7-naphthyridin-3-yl)cydopropanecarboxamide

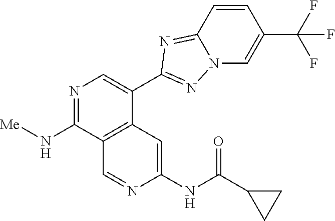 [SMILES: CNc1ncc(-c2nc3ccc(C(F)(F)F)cn3n2)c2cc(NC(=O)C3CC3)ncc12]

N-(8-(methylamino)-5-(6-(trifluoromethyl)-[1,2,4]triazolo[1,5-a]pyridin-2-yl)-2,7-naphthyridin-3-yl)cyclopropanecarboxamide was synthesized using a similar procedure that was previously described in Example 470 by using N-(8-(methylamino)-5-(4,4,5,5-tetramethyl-1,3,2-dioxaborolan-2-yl)-2,7-naphthyridin-3-yl)cyclopropanecarboxamide and 2-chloro-6-(trifluoromethyl)-[1,2,4]triazolo[1,5-a]pyridine as the starting material. LCMS (ESI) m/z 428.1, [M+H]⁺. ¹H NMR (300 MHz, DMSO-d₆) δ 10.95 (s, 1H), 9.61-9.5 (m, 1H), 9.49 (s, 1H), 9.43 (s, 1H), 8.86 (s, 1H), 8.41-8.34 (m, 1H), 8.09-8.02 (m, 1H), 7.98-7.93 (m, 1H), 3.08 (d, J=4.4 Hz, 3H), 2.16-2.01 (m, 1H), 0.92-0.78 (m, 4H).

### Example 497: Synthesis of NV (8-(methylamino)-5-(6-(methylsulfanyl)-[1,2,4]triazolo[1,5-a]pyridin-2-yl)-2,7-naphthyridin-3-yl)cyclopropanecarboxamide

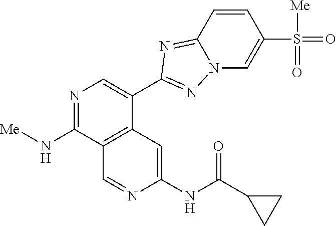 [SMILES: CNc1ncc(-c2nc3ccc(S(C)(=O)=O)cn3n2)c2cc(NC(=O)C3CC3)ncc12]

### Step 1: ethyl N-[(5-methanesulfonylpyridin-2-yl)carbamothioyl]carbamate

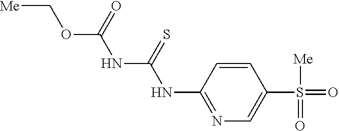 [SMILES: CCOC(=O)NC(=S)Nc1ccc(S(C)(=O)=O)cn1]

A solution of 5-methanesulfonylpyridin-2-amine (500.3 mg; 2.901 mmol; 1.00 eq.) and O-ethyl carbonisothiocyanatidate (420.1 mg; 3.205 mmol; 1.10 eq.) in 1,4-dioxane (5 mL) was stirred at room temperature overnight under nitrogen atmosphere. The desired product was detected via LCMS. The resulting mixture was concentrated under reduced pressure. The residue was purified by flash chromatography on silica gel column using 10-20% of EtOAc in petroleum ether as eluent to provide ethyl N-[(5-methanesulfonylpyridin-2-yl)carbamothioyl]carbamate as a yellow solid (750.2 mg, 85.1%). LCMS (ESI) m/z 304.0, [M+H]⁺.

### Step 2: 6-(methylsulfonyl)-[1,2,4]triazolo[1,5-a]pyridin-2-amine

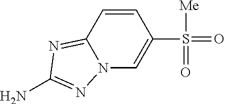 [SMILES: CS(=O)(=O)c1ccc2nc(N)nn2c1]

To a stirring mixture of ethyl N-[(5-methanesulfonylpyridin-2-yl)carbamothioyl]carbamate (700.1 mg; 2.300 mmol; 1.00 eq.) and hydroxylamine hydrochloride (239.2 mg; 3.434 mmol; 1.50 eq.) in MeOH/EtOH (1:1, 5 mL) was added DIPEA (447.3 mg; 3.454 mmol; 1.50 eq.). The resulting mixture was stirred at 60° C. for 4 hours. The desired product was detected via LCMS. The precipitated solids were collected by filtration and washed with MeOH (1 mL) to afford 6-methanesulfonyl-[1,2,4]triazolo[1,5-a]pyridin-2-amine as a white solid (320.2 mg, 65.3%). LCMS (ESI) m/z 213.0, [M+H]⁺.

### Step 3: 2-chloro-6-(methylsulfonyl)-[1,2,4]triazolo[1,5-a]pyridine

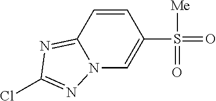 [SMILES: CS(=O)(=O)c1ccc2nc(Cl)nn2c1]

To a stirred solution of 6-methanesulfonyl-[1,2,4]triazolo[1,5-a]pyridin-2-amine (150.0 mg; 0.701 mmol; 1.00 eq.) and CuCl₂ (28.3 mg; 0.211 mmol; 1.00 eq.) in HCl (12 M in water, 2 mL) was added a solution of NaNO₂ (58.2 mg; 0.841 mmol, 1.19 eq.) in water (0.5 mL) dropwise at 0° C. The mixture was stirred at room temperature for 1 hour. The desired product was detected via LCMS. The reaction was diluted with water (20 mL) and neutralized to pH 7 with a saturated NaHCO₃ aqueous solution. The resulting mixture was extracted with EtOAc (3×20 mL). The combined organic layers were washed with brine (3×10 mL), dried over anhydrous Na₂SO₄, filtered, and concentrated under reduced pressure. The residue was purified by flash chromatography on silica gel column using 5-8% of MeOH in CH₂Cl₂ as eluent to provide 2-chloro-6-methanesulfonyl-[1,2,4]triazolo[1,5-a]pyridine as a white solid (60.1 mg, 36.6%). LCMS (ESI) m/z 232.0, [M+H]⁺.

### Step 4: N-(8-(methylamino)-5-(6-(methylsulfonyl)-[1,2,4]triazolo[1,5-a]pyridin-2-yl)-2,7-naphthyridin-3-yl)cyclopropanecarboxamide

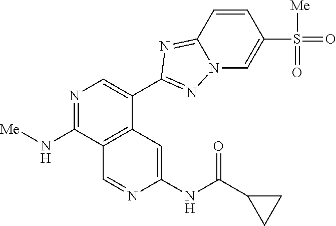 [SMILES: CNc1ncc(-c2nc3ccc(S(C)(=O)=O)cn3n2)c2cc(NC(=O)C3CC3)ncc12]

N-(8-(methylamino)-5-(6-(methylsulfonyl)-[1,2,4]triazolo[1,5-a]pyridin-2-yl)-2,7-naphthyridin-3-yl)cyclopropanecarboxamide was synthesized using a similar procedure that was previously described in Example 470, by using N-(8-(methylamino)-5-(4,4,5,5-tetramethyl-1,3,2-dioxaborolan-2-yl)-2,7-naphthyridin-3-yl)cyclopropanecarboxamide and 2-chloro-6-(methylsulfonyl)-[1,2,4]triazolo[1,5-a]pyridine as the starting material. LOVES (ESI) m/z 438.1, [M+H]⁺. ¹H NMR (300 MHz, DMSO-d₆) δ 10.97 (s, 1H), 9.63-9.59 (m, 1H), 9.42 (s, 1H), 9.37 (s, 1H), 8.94 (s, 1H), 8.45-8.40 (m, 1H), 8.09-8.02 (m, 2H), 3.42 (s, 3H), 3.05 (d, J=4.4 Hz, 3H), 2.12-2.02 (m, 1H), 0.91-0.81 (m, 4H).

### Example 498: Synthesis of N-(5-(6-(2-cyano-2-methylpropoxy)-[1,2,4]triazolo[1,5-a]pyridin-2-yl)-8-(methylamino)-2,7-naphthyridin-3-yl)cyclopropanecarboxamide

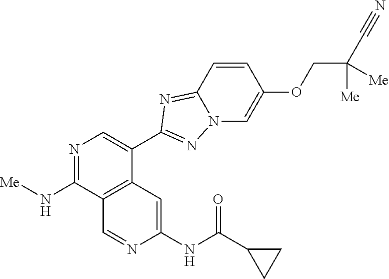 [SMILES: CNc1ncc(-c2nc3ccc(OCC(C)(C)C#N)cn3n2)c2cc(NC(=O)C3CC3)ncc12]

### Step 1: 2,2-dimethyl-3-((6-nitropyridin-3-yl)oxy)propanenitrile

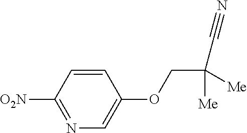 [SMILES: CC(C)(C#N)COc1ccc([N+](=O)[O-])nc1]

To a stirred solution of 3-hydroxy-2,2-dimethylpropanenitrile (523.1 mg; 5.272 mmol; 1.00 eq.) in THF (8 mL) was added NaH (60%) (465.1 mg; 11.622 mmol; 2.50 eq.) in portions at 0° C. After stirring at room temperature for 0.5 hour, to the mixture was added 5-fluoro-2-nitropyridine (830.2 mg; 5.843 mmol; 1.1.0 eq.) at 0° C. The resulting solution was stirred at room temperature for 1 hour under nitrogen atmosphere. The desired product was detected via LCMS. The reaction was quenched with a saturated NH₄Cl aqueous solution (30 mL) and extracted with EtOAc (3×20 mL). The combined organic layers were washed with brine (3×10 mL), dried over anhydrous Na₂SO₄, filtered, and concentrated under reduced pressure. The residue was purified by flash chromatography on silica gel column using 10-15% of EtOAc in petroleum ether as eluent to provide 2,2-dimethyl-3-((6-nitropyridin-3-yl)oxy)propanenitrile as a yellow solid (1.01 g, 85.6%). LCMS (ESI) m/z 222.1, [M+H]⁺.

### Step 2: 3-((6-aminopyridin-3-yl)oxy)-2,2-dimethylpropanenitrile

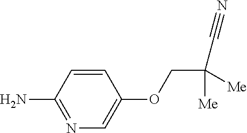 [SMILES: CC(C)(C#N)COc1ccc(N)nc1]

To a stirring mixture of 2,2-dimethyl-3-((6-nitropyridin-3-yl)oxy)propanenitrile (800.0 mg; 3.610 mmol; 1.00 eq.) in MeOH (10 mL) added 10% Pd/C (260.1 mg; 30% w/w) under nitrogen atmosphere. The mixture was hydrogenated at 40° C. for 1 hour under hydrogen atmosphere (2 atm). The desired product was detected via LCMS. The resulting mixture was filtered, the filter cake was washed with MeOH. The filtrate was concentrated under reduced pressure to afford 3-((6-aminopyridin-3-yl)oxy)-2,2-dimethylpropanenitrile as a yellow oil (590.1 mg, 85.3%). LCMS (ESI) m/z 192.1, [M+H]⁺.

### Step 3: ethyl N-{[5-(2-cyano-2,2-dimethylethoxy)pyridin-2-yl]carbamothioyl}carbamate

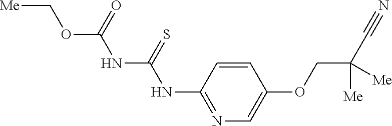 [SMILES: CCOC(=O)NC(=S)Nc1ccc(OCC(C)(C)C#N)cn1]

A solution of 3-((6-aminopyridin-3-yl)oxy)-2,2-dimethylpropanenitrile (590.1 mg; 3.081 mmol; 1.00 eq.) and O-ethyl carbonisothiocyanatidate (445.2 mg; 3.394 mmol; 1.10 eq.) in 1,4-dioxane (6 mL) was stirred at room temperature overnight under nitrogen atmosphere. The desired product was detected via LCMS. The resulting mixture was concentrated under reduced pressure to afford ethyl N-{[5-(2-cyano-2,2-dimethylethoxy)pyridin-2-yl]carbamothioyl}carbamate as a yellow solid (950.2 mg, crude). LCMS (ESI) m/z 323.1, [M+H]⁺.

### Step 4: 3-((2-amino-[1,2,4]triazolo[1,5-a]pyridin-6-yl)oxy)-2,2-dimethylpropanenitrile

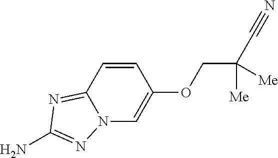 [SMILES: CC(C)(C#N)COc1ccc2nc(N)nn2c1]

To a solution of ethyl N-{[5-(2-cyano-2,2-dimethylethoxy)pyridin-2-yl]carbamothioyl}carbamate (950.2 mg; 3.722 mmol; 1.00 eq.) and hydroxylamine hydrochloride (384.3 mg; 5.512 mmol; 1.50 eq.) in MeOH/EtOH. (1:1, 5 mL) was added DIPEA (720.1 mg; 5.572 mmol; 1.50 eq.). The resulting mixture was stirred at 60° C. for 3 hours. LCMS indicated that the starting material was consumed. The mixture was allowed to cool down to room temperature. The resulting mixture was concentrated under reduced pressure. The residue was purified by flash chromatography, on silica gel column, using 5-8% of MeOH in 01202 as eluent to provide 3-((2-amino-[1,2,4]triazolo[1,5-a]pyridin-6-yl)oxy)-2,2-dimethylpropanenitrile as a white solid (500.1 mg, 58.0%). LCMS (ESI) m/z 232.1, [M+H]⁺.

### Step 5: 3-((2-chloro-[1,2,4]triazolo[1,5-a]pyridin-6-yl)oxy)-2,2-dimethylpropanenitrile

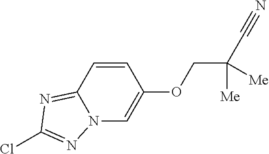 [SMILES: CC(C)(C#N)COc1ccc2nc(Cl)nn2c1]

To a stirred solution of 3-((2-amino-[1,2,4]triazolo[1,5-a]pyridin-6-yl)oxy)-2,2-dimethylpropanenitrile (400.3 mg; 1.730 mmol; 1.00 eq.) and CuCl₂ (69.2 mg; 0.513 mmol; 0.30 eq.) in HCl (12 M: in water, 4 mL) was added a solution of NaNO₂ (1.444 mg; 2.082 mmol; 1.20 eq.) in water (1 mL) dropwise at 0° C. The resulting solution was stirred at room temperature for 1 hour. The desired product was detected via LCMS. The mixture was neutralized to pH=7 with a saturated Na₂CO₃ aqueous solution. The resulting mixture was extracted with EtOAc (3×20 mL). The combined organic layers were washed with brine (3×10 mL), dried over anhydrous Na₂SO₄, filtered, and concentrated under reduced pressure to afford 3-((2-chloro-[1,2,4]triazolo[1,5-a]pyridin-6-yl)oxy)-2,2-dimethylpropanenitrile as a white solid (400.1 mg, 92.2%). LCMS (ESI) m/z 251.1, [M+H]⁺.

### Step 6: N-(5-(6-(2-cyano-2-methylpropoxy)-[1,2,4]triazolo[1: a]pyridin-2-yl)-8-(methylamino)-2,7-naphthyridin-3-yl)cyclopropanecarboxamide

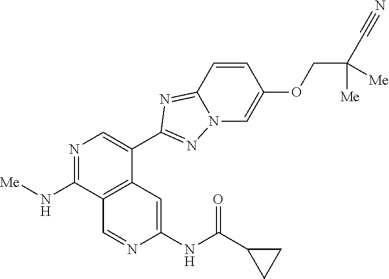 [SMILES: CNc1ncc(-c2nc3ccc(OCC(C)(C)C#N)cn3n2)c2cc(NC(=O)C3CC3)ncc12]

N-(5-(6-(2-cyano-2-methylpropoxy)-[1,2,4]triazolo[1,5-a]pyridin-2-yl)-8-(methylamino)-2,7-naphthyridin-3-yl)cyclopropanecarboxamide was synthesized using a similar procedure that was previously described in Example 470, by using N-(8-(methylamino)-5-(4,4,5,5-tetramethyl-1,3,2-dioxaborolan-2-yl)-2,7-naphthyridin-3-yl)cyclopropanecarboxamide and 3-((2-chloro-[1,2,4]triazolo[1,5-a]pyridin-6-yl)oxy)-2,2-dimethylpropanenitrile as the starting material. LCMS (ESI) m/z 457.2, [M+H]⁺. ¹H NMR (400 MHz, DMSO-d₆) δ 10.94 (s, 1H), 9.42 (s, 1H), 9.41 (s, 1H), 8.77 (d, J=2.0 Hz, 1H), 8.76 (s, 1H), 8.32-8.21 (m, 1H), 7.81 (d, J=9.6 Hz, 1H), 7.55 (d, J=9.6, 2.0 Hz, 1H), 4.19 (s, 2H), 3.06 (d, J=4.4 Hz, 3H), 2.13-2.03 (m, 1H), 1.47 (s, 6H), 0.90-0.78 (m, 4H).

### Example 499: Synthesis of N-(5-(7-fluoro-6-(2-methylmorpholino)-[1,2,4]triazolo[1,5-a]pyridin-2-yl)-8-(methylamino)-2,7-naphthyridin-3-yl)cyclopropanecarboxamide

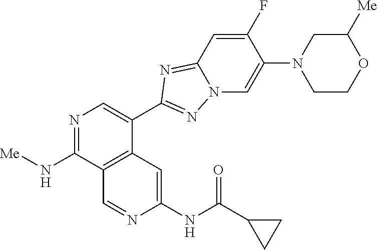 [SMILES: CNc1ncc(-c2nc3cc(F)c(N4CCOC(C)C4)cn3n2)c2cc(NC(=O)C3CC3)ncc12]

### Step 1: O-ethyl N-((5-bromo-4-fluoropyridin-2-yl)carbamothioyl)carbamate

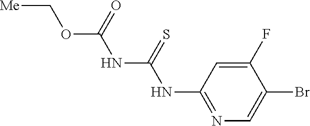 [SMILES: CCOC(=O)NC(=S)Nc1cc(F)c(Br)cn1]

A solution of 5-bromo-4-fluoropyridin-2-amine (1.00 g; 5.236 mmol; 1.00 eq.) and O-ethyl carbonisothiocyanatidate (0.76 g; 5.760 mmol; 1.10 eq.) in 1,4-dioxane (5 mL) was stirred at room temperature overnight under nitrogen atmosphere. LCMS indicated that the starting material was consumed. The resulting mixture was concentrated under reduced pressure to afford O-ethyl. N-((5-bromo-4-fluoropyridin-2-yl)carbamothioyl)carbamate as a orange solid (1.50 g, crude). LCMS (ESI) m/z 322.0, [M+H]⁺.

### Step 2: 6-bromo-7-fluoro-[1,2,4]triazolo[1,5-a]pyridin-2-amine

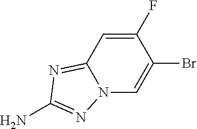 [SMILES: Nc1nc2cc(F)c(Br)cn2n1]

To a stirred solution of O-ethyl N-((5-bromo-4-fluoropyridin-2-yl)carbamothioyl)carbamate (2.50 g; 7.760 mmol; 1.00 eq.) and hydroxylamine hydrochloride (0.81 g; 11.640 mmol; 1.50 eq.) in a mixture solvent of MeOH/EtOH (1:1, 6 mL) was added DIPEA (1.50 g; 11.640 mmol; 1.50 eq.) at room temperature. The resulting mixture was stirred at 60° C. for 2 hours under nitrogen atmosphere. The desired product was detected via LCMS. The mixture was allowed to cool down to room temperature. The resulting mixture was concentrated under reduced pressure. The residue was purified by flash chromatography, on silica gel column, using 5-8% of MeOH in CH₂Cl₂ as eluent to provide 6-bromo-7-fluoro-[1,2,4]triazolo[1,5-a]pyridin-2-amine as a brown solid (1.20 g, 66.9%). LCMS (ESI) m/z 231.0, [M+H]⁺.

### Step 3: 6-bromo-2-chloro-7-fluoro-[1,2,4]triazolo[1,5-a]pyridine

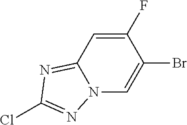 [SMILES: Fc1cc2nc(Cl)nn2cc1Br]

To a stirred solution of 6-bromo-7-fluoro-[1,2,4]triazolo[1,5-a]pyridin-2-amine (400.0 mg; 1.731 mmol; 1 eq.) in HCl (12 M in water, 5 mL) were added CuCl₂ (69.8 mg; 0.519 mmol; 0.30 eq.) and a solution of NaNO₂ (179.2 mg; 2.597 mmol; 1.50 eq.) in water (1 mL) at 0° C. The resulting mixture was stirred at room temperature for 1 hour under nitrogen atmosphere. The reaction was completed by LCMS and was diluted with water (20 mL) and neutralized to pH=7 with a saturated NaHCO₃ solution. The mixture was extracted with EtOAc (3×50 mL), the combined organic layers were washed with brine (3×20 mL), dried over anhydrous Na₂SO₄, filtered, and concentrated under reduced pressure to afford 6-bromo-2-chloro-7-fluoro-[1,2,4]triazolo[1,5-a]pyridine as an off-white solid (150.0 mg, 34.6%). LCMS (ESI) m/z 249.9, [M+H]⁺.

### Step 4: 4-(2-chloro-7-fluoro-[1,2,4]triazolo[1,5-a]pyridin-6-yl)-2-methylmorpholine

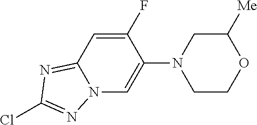 [SMILES: CC1CN(c2cn3nc(Cl)nc3cc2F)CCO1]

To a mixture of 6-bromo-2-chloro-7-fluoro-[1,2,4]triazolo[1,5-a]pyridine (100.0 mg; 0.399 mmol; 1.00 eq.) and 2-methylmorpholine (44.4 mg; 0.439 mmol; 1.10 eq.) in 1,4-dioxane (2 mL) were added Pd-PEPPSI-IHeptCl 3-chloropyridine (38.9 mg; 0.040 mmol; 0.10 eq.), Cs₂CO₃ (390.3 mg; 1.197 mmol; 3.00 eq.). The resulting mixture was stirred at 100° C. for 3 hours under nitrogen atmosphere. The desired product was detected via LCMS. The mixture was allowed to cool down to room temperature. The resulting mixture was concentrated under reduced pressure. The residue was purified by flash chromatography, on silica gel column, using 30-50% of EtOAc in petroleum ether as eluent to provide 4-(2-chloro-7-fluoro-[1,2,4]triazolo[1,5-a]pyridin-6-yl)-2-methylmorpholine as a light yellow solid (70.0 mg, 64.8%). LCMS (ESI) m/z 271.1, [M+H]⁺.

### Step 5: N-(5-(7-fluoro-6-(2-methylmorpholino)-[1,2,4]triazolo[1,5-a]pyridin-2-yl)-8-(methylamino)-2,7-naphthyridin-3-yl)cyclopropanecarboxamide

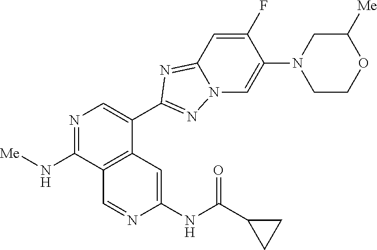 [SMILES: CNc1ncc(-c2nc3cc(F)c(N4CCOC(C)C4)cn3n2)c2cc(NC(=O)C3CC3)ncc12]

N-(5-(7-fluoro-6-(2-methylmorpholino)-[1,2,4]triazolo[1,5-a]pyridin-2-yl)-8-(methylamino)-2,7-naphthyridin-3-yl)cyclopropanecarboxamide was synthesized using a similar procedure that was previously described in Example 470, by using N-(8-(methylamino)-5-(4,4,5,5-tetramethyl-1,3,2-dioxaborolan-2-yl)-2,7-naphthyridin-3-yl)cyclopropanecarboxamide and 4-(2-chloro-7-fluoro-[1,2,4]triazolo[1,5-a]pyridin-6-yl)-2-methylmorpholine as the starting material. LCMS (ESI) m/z 477.2, [M+H]⁺. ¹H NMR (400 MHz, DMSO-d₆) δ 10.93 (s, 1H), 9.40 (s, 1H), 9.36 (s, 1H), 8.73 (s, 1H), 8.60 (d, J=7.6 Hz, 1H), 8.32-8.24 (m, 1H), 7.81 (d, J=12.0 Hz, 1H), 3.96-3.88 (m, 1H), 3.79-3.67 (m, 2H), 3.31-3.18 (m, 2H), 3.05 (d, J=4.4 Hz, 3H), 2.91-2.80 (m, 1H), 2.61-2.54 (m, 1H), 2.13-2.02 (m, 1H), 1.16 (d, J=6.0 Hz, 3H), 0.91-0.79 (m, 4H).

### Example 500: Synthesis of N-(5-(6-(benzyloxy)-[1,2,4]triazolo[1,5-a]pyridin-2-yl)-8-(methylamino)-2,7-naphthyridin-3-yl)cyclopropanecarboxamide

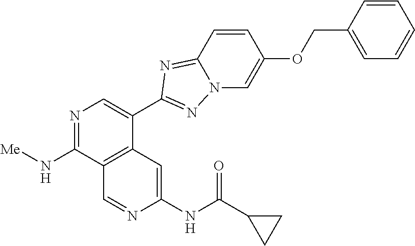 [SMILES: CNc1ncc(-c2nc3ccc(OCc4ccccc4)cn3n2)c2cc(NC(=O)C3CC3)ncc12]

### Step 1: ethyl N-{[5-(benzyloxy)pyridin-2-yl]carbamothioyl}carbamate

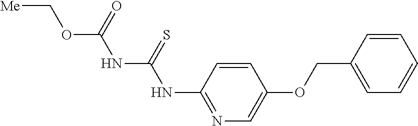 [SMILES: CCOC(=O)NC(=S)Nc1ccc(OCc2ccccc2)cn1]

A solution of 5-(benzyloxy)pyridin-2-amine (1.00 g; 4.990 mmol; 1.00 eq.) and O-ethyl carbonisothiocyanatidate (0.72 g; 5.490 mmol; 1.10 eq.) in 1,4-dioxane (10 mL) was stirred at room temperature overnight under nitrogen atmosphere. LCMS indicated that starting material was consumed. The resulting mixture was concentrated under reduced pressure to afford ethyl N-{[5-(benzyloxy)pyridin-2-yl]carbamothioyl}carbamate as a brown solid (1.72 g, crude). LCMS (ESI) m/z 332.1, [M+H]⁺

### Step 2: 6-(benzyloxy)-[1,2,4]triazolo[1,5-a]pyridin-2-amine

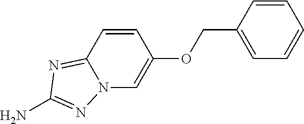 [SMILES: Nc1nc2ccc(OCc3ccccc3)cn2n1]

To a solution of ethyl N-{[5-(benzyloxy)pyridin-2-yl]carbamothioyl}carbamate (1.20 g; 3.621 mmol; 1.00 eq.) in a mixture solvent of EtOH/MeOH. (1:1, 6 mL) were added NH₂OH·HCl (0.38 g; 5.431 mmol; 1.50 eq.) and DIPEA (1.01 g; 7.834 mmol; 2.00 eq.) at room temperature. The mixture was stirred at 60° C. overnight under nitrogen atmosphere. The reaction was completed by LCMS. The resulting mixture was concentrated under reduced pressure. The residue was purified by flash chromatography, on silica gel column, using 0-10% of MeOH in CH₂Cl₂ as eluent to provide 6-(benzyloxy)-[1,2,4]triazolo[1,5-a]pyridin-2-amine as a off-white solid (530.0 mg, 60.9%). LCMS (ESI) m/z 241.1, [M+H]⁺.

### Step 3: 6-(benzyloxy)-2-chloro-[1,2,4]triazolo[1,5-a]pyridine

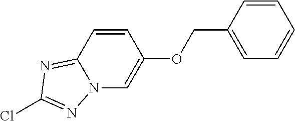 [SMILES: Clc1nc2ccc(OCc3ccccc3)cn2n1]

To a stirred solution of 6-(benzyloxy)-[1,2,4]triazolo[1,5-a]pyridin-2-amine (530.0 mg; 2.223 mmol; 1.00 eq.) and CuCl₂ (89.6 mg; 0.667 mmol; 0.30 eq.) in HCl (12 M in water, 4 mL) was added a solution of NaNO₂ (230.0 mg; 3.334 mmol; 1.50 eq.) in water (1 mL) dropwise at 0° C. The resulting mixture was stirred at room temperature for 2 hours under nitrogen atmosphere. The desired product was detected via LCMS. The mixture was neutralized to pH===7 with NaOH solution and precipitate solids were formed. The precipitated solids were collected by filtration and washed with water (3×3 mL) to provide 6-(benzyloxy)-2-chloro-[1,2,4]triazolo[1,5-a]pyridine as a grey solid (480.0 mg, 83.1%). LCMS (ESI) m/z 260.1, [M+H]⁺.

### Step 4: N-(5-(6-(benzyloxy)-[1,2,4]triazolo[1,5-a]pyridin-2-yl)-8-(methylamino)-2,7-naphthyridin-3-yl)cyclopropanecarboxamide

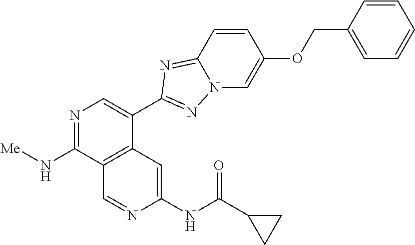 [SMILES: CNc1ncc(-c2nc3ccc(OCc4ccccc4)cn3n2)c2cc(NC(=O)C3CC3)ncc12]

N-(5-(6-(benzyloxy)-[1,2,4]triazolo[1,5-a]pyridin-2-yl)-8-(methylamino)-2,7-naphthyridin-3-yl)cyclopropanecarboxamide was synthesized using a similar procedure that was previously described in Example 470, by using N-(8-(methylamino)-5-(4,4,5,5-tetramethyl-1,3,2-1.0 dioxaborolan-2-yl)-2,7-naphthyridin-3-yl)cyclopropanecarboxamide and 6-(benzyloxy)-2-chloro-[1,2,4]triazolo[1,5-a]pyridine as the starting material. LCMS (ESI) m/z 466.2, [M+H]⁺. ¹H NMR (400 MHz, DMSO-d₆) δ 10.92 (s, 1H), 9.41 (s, 1H), 9.40 (s, 1H), 8.77 (d, J=1.6 Hz, 1H), 8.73 (s, 1H), 8.30-8.20 (m, 1H), 7.79 (d, J=9.6 Hz, 1H), 7.58-7.49 (m, 3H), 7.49-7.32 (m, 3H), 5.24 (s, 2H), 3.06 (d, J=4.4 Hz, 3H), 2.13-2.01 (m, 1H), 0.93-0.75 (m, 4H).

### Example 501: Synthesis of N-(5-(6-(2-hydroxy-2-methylpropoxy)-[1,2,4]triazolo[1,5-a]pyridin-2-yl)-8-(methylamino)-2,7-naphthyridin-3-yl)cyclopropanecarboxamide

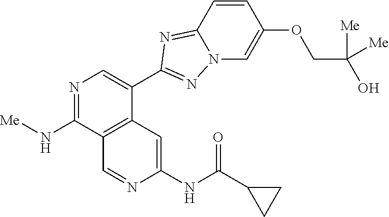 [SMILES: CNc1ncc(-c2nc3ccc(OCC(C)(C)O)cn3n2)c2cc(NC(=O)C3CC3)ncc12]

### Step 1: 2-methyl-1-((6-nitropyridin-3-yl)oxy)propan-2-ol

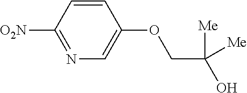 [SMILES: CC(C)(O)COc1ccc([N+](=O)[O-])nc1]

To a stirred solution of 2-methylpropane-1,2-diol (0.70 g; 7.742 mmol; 1.10 eq.) in THF (5 mL) was added NaH (60%) (0.20 g; 8.446 mmol; 1.20 eq.) in portions at 0° C. After stirring at room temperature for 0.5 hour under nitrogen atmosphere, To the above solution was 5-fluoro-2-nitropyridine (1.00 g; 7.038 mmol; 1.00 eq.) at 0° C. The resulting solution was stirred at room temperature for 2 hours. The desired product was detected via LCMS. The reaction was quenched with saturated NH₄Cl solution (20 mL) and extracted with EtOAc (3×30 mL). The combined organic layers were washed with brine (3×10 mL), dried over anhydrous Na₂SO₄, filtered, and concentrated under reduced pressure to afford 2-methyl-1-((6-nitropyridin-3-yl)oxy)propan-2-ol as a brown oil (1.00 g, 67.0%). LCMS (ESI) m/z 213.1, [M+H]⁺.

### Step 2: 1-((6-aminopyridin-3-yl)oxy)-2-methylpropan-2-ol

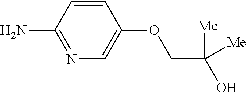 [SMILES: CC(C)(O)COc1ccc(N)nc1]

To a solution of 2-methyl-1-((6-nitropyridin-3-yl)oxy)propan-2-ol (500.0 mg; 2.545 mmol; 1.00 eq.) in MeOH (10 mL) was added 10% Pd/C (100.0 mg; 20% w/w) under nitrogen atmosphere. The mixture was hydrogenated at 40° C. for 1 hour under hydrogen atmosphere (2 atm). The desired product was detected via LCMS. The resulting mixture was filtered, the filter cake was washed with MeOH. The filtrate was concentrated under reduced pressure to afford 1-((6-aminopyridin-3-yl)oxy)-2-methylpropan-2-ol as a colorless oil (405.0 mg, 87.3%). LCMS (ESI) m/z 183.1, [M+H]⁺.

### Step 3: ethyl N-{[5-(2-hydroxy-2-methylpropoxy)pyridin-2-yl]carbamothioyl}carbamate

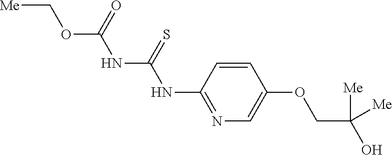 [SMILES: CCOC(=O)NC(=S)Nc1ccc(OCC(C)(C)O)cn1]

A mixture of 1-((6-aminopyridin-3-yl)oxy)-2-methylpropan-2-ol (400.0 mg; 2.1.95 mmol; 1.00 eq.) and O-ethyl carbonisothiocyanatidate (316.7 mg; 2.414 mmol; 1.10 eq.) in 1,4-dioxane (10 mL) was stirred at room temperature overnight under nitrogen atmosphere. The desired product was detected via LCMS. The resulting mixture was concentrated under reduced pressure to afford ethyl N-{[5-(2-hydroxy-2-methylpropoxy)pyridin-2-yl]carbamothioyl}carbamate as a orange solid (600.0 mg, crude). LCMS (ESI) m/z 314.1, [M+H]⁺.

### Step 4: 1-((2-amino-[1,2,4]triazolo[1,5-a]pyridin-6-yl)oxy)-2-methylpropan-2-ol

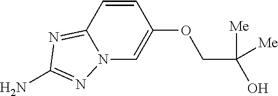 [SMILES: CC(C)(O)COc1ccc2nc(N)nn2c1]

To a solution of ethyl N-{[5-(2-hydroxy-2-methylpropoxy)pyridin-2-yl]carbamothioyl}carbamate (500.0 mg; 1.596 mmol; 1.00 eq.) and hydroxylamine hydrochloride (166.3 mg; 2.394 mmol; 1.50 eq.) in a mixture solvent of EtOH/MeOH (1:1, 10 mL) was added DIPEA (309.3 mg; 2.394 mmol; 1.50 eq.) at room temperature. The resulting mixture was stirred at 60° C. for 2 hours under nitrogen atmosphere. LCMS indicated that the starting material was consumed. The mixture was allowed to cool down to room temperature. The resulting mixture was concentrated under reduced pressure. The residue was purified by flash chromatography, on silica gel column, using 5-8% of MeOH in CH₂Cl₂ as eluent to provide 1-((2-amino-[1,2,4]triazolo[1,5-a]pyridin-6-yl)oxy)-2-methylpropan-2-ol as a light yellow solid (280.0 mg, 79.0%). LCMS (ESI) m/z 223.1, [M+H]⁺.

### Step 5: 1-((2-chloro-[1,2,4]triazolo[1,5-a]pyridin-6-yl)oxy)-2-methylpropan-2-ol

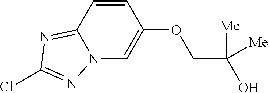 [SMILES: CC(C)(O)COc1ccc2nc(Cl)nn2c1]

To a stirred solution of 1-((2-amino-[1,2,4]triazolo[1,5-a]pyridin-6-yl)oxy)-2-methylpropan-2-ol (300.0 mg; 1.350 mmol; 1.00 eq.) in HCl (12 M in water, 4 mL) was added CuCl₂ (54.4 mg; 0.405 mmol; 0.30 eq.) and a solution of NaNO₂ (139.7 mg; 2.025 mmol; 1.50 eq.) in water (1 mL) at 0° C. The resulting solution was stirred at room temperature for 1 hour under nitrogen atmosphere. The desired product was detected via LCMS. The resulting mixture was diluted with water (20 mL) and neutralized to pH=7 with a saturated NaHCO₃ aqueous solution. The resulting mixture was extracted with EtOAc (3×50 mL). The combined organic layers were washed with brine (3×20 mL), dried over anhydrous Na₂SO₄, filtered, and concentrated under reduced pressure to afford 1-((2-chloro-[1,2,4]triazolo[1,5-a]pyridin-6-yl)oxy)-2-methylpropan-2-ol as an off-white solid (120.0 mg, 36.8%). LCMS (ESI) m/z 242.1, [M+H]⁺.

### Step 6: N-(5-(6-(2-hydroxy-2-methylpropoxy)-[1,2,4]triazolo[1,5-a]pyridin-2-yl)-8-(methylamino)-2,7-naphthyridin-3-yl)cyclopropanecarboxamide

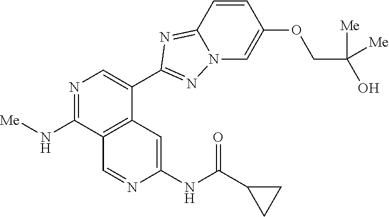 [SMILES: CNc1ncc(-c2nc3ccc(OCC(C)(C)O)cn3n2)c2cc(NC(=O)C3CC3)ncc12]

N-(5-(6-(2-hydroxy-2-methylpropoxy)-[1,2,4]triazolo[1,5-a]pyridin-2-yl)-8-(methylamino)-2,7-naphthyridin-3-yl)cyclopropanecarboxamide was synthesized using a similar procedure that was previously described in Example 470 by using N-(8-(methylamino)-5-(4,4,5,5-tetramethyl-1,3,2-dioxaborolan-2-yl)-2,7-naphthyridin-3-yl)cyclopropanecarboxamide and 1-((2-chloro-[1,2,4]triazolo[1,5-a]pyridin-6-yl)oxy)-2-methyl propan-2-ol as the starting material. LCMS (ESI) m/z 448.2, [M+H]⁺. ¹H NMR (400 MHz, DMSO-d₆) δ 10.96 (s, 1H), 9.43 (s, 2H), 8.70 (s, 1H), 8.66 (d, J=2.0 Hz, 1H), 8.48-8.35 (m, 1H), 7.77 (d, J=9.6 Hz, 1H), 7.49 (dd, J=9.6, 2.0 Hz, 1H), 4.71 (s, 1H), 3.88 (s, 2H), 3.07 (d, J=4.4 Hz, 3H), 2.13-2.02 (m, 1H), 1.25 (s, 6H), 0.91-0.78 (m, 4H).

### Example 502: Synthesis of N-(5-(6-(7-methyl-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide

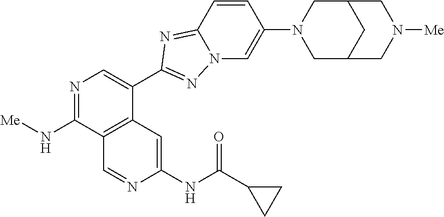 [SMILES: CNc1ncc(-c2nc3ccc(N4CC5CC(CN(C)C5)C4)cn3n2)c2cc(NC(=O)C3CC3)ncc12]

### Step 1: tert-butyl 7-(2-chloro-[1,2,4]triazolo[1,5-a]pyridin-6-yl)-3,7-diazabicyclo[3.3.1]nonane-3-carboxylate

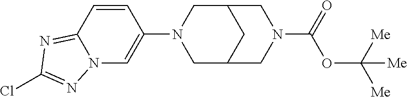 [SMILES: CC(C)(C)OC(=O)N1CC2CC(C1)CN(c1ccc3nc(Cl)nn3c1)C2]

To a solution of 6-bromo-2-chloro-[1,2,4]triazolo[1,5-a]pyridine (180.0 mg; 0.774 mmol; 1.00 eq.) and tert-butyl 3,7-diazabicyclo[3.3.1]nonane-3-carboxylate (175.2 mg; 0.774 mmol; 1.00 eq.) in 1,4-dioxane (10 mL) were added Cs₂CO₃ (1.00 g; 3.097 mmol; 4.00 eq.) and Pd-PEPPSI-IHeptCl 3-chloropyridine (75.4 mg; 0.077 mmol; 0.10 eq.). The resulting mixture was stirred at 100° C. for 3 hours under nitrogen atmosphere. The desired product was detected via LCMS. The solvent was concentrated under vacuum. The residue was purified by flash chromatography, on silica gel column, using 0-15% of MeOH: in CH₂Cl₂ as eluent to provide tert-butyl 7-(2-chloro-[1,2,4]triazolo[1,5-a]pyridin-6-yl)-3,7-diazabicyclo[3.3.1]nonane-3-carboxylate as a yellow solid (150.0 mg, 48.7%). LCMS (ESI) m/z 378.2, [M+H]⁺.

### Step 2: tert-butyl 7-(2-(6-(cyclopropanecarboxamido)-1-(methylamino)-2,7-naphthyridin-4-yl)-[1,2,4]triazolo[1,5-a]pyridin-6-yl)-3,7-diazabicyclo[3.3.1]nonane-3-carboxylate

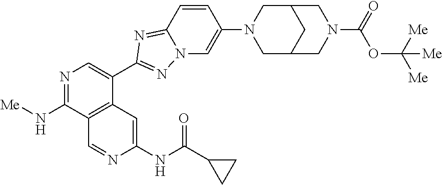 [SMILES: CNc1ncc(-c2nc3ccc(N4CC5CC(CN(C(=O)OC(C)(C)C)C5)C4)cn3n2)c2cc(NC(=O)C3CC3)ncc12]

To a solution of tert-butyl 7-(2-chloro-[1,2,4]triazolo[1,5-a]pyridin-6-yl)-3,7-diazabicyclo[3.3.1]nonane-3-carboxylate (140.0 mg; 0.370 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (137.6 mg; 0.374 mmol; 1.00 eq.) in a mixture solvent of 1,4-dioxane/water (5:1, 10 mL) were added XPhos Pd G₃ (94.2 mg; 0.112 mmol; 0.30 eq.), XPhos (53.0 mg; 0.112 mmol; 0.30 eq.) and K₃PO₄ (236.8 mg; 1.116 mmol; 3.00 eq.). The resulting mixture was stirred at 110° C. for 1 hour under nitrogen atmosphere. The desired product was detected via LCMS. The solvent was concentrated under vacuum. The residue was purified by flash chromatography on silica gel column using 0-15% of MeOH in CH₂Cl₂ as eluent to provide tert-butyl 7-(2-(6-(cyclopropanecarboxamido)-1-(methylamino)-2,7-naphthyridin-4-yl)-[1,2,4]triazolo[1,5-a]pyridin-6-yl)-3,7-diazabicyclo[3.3.1]nonane-3-carboxylate as a yellow solid (90.0 mg, 39.5%). LCMS (ESI) m/z 584.3, [M+H]⁺.

### Step 3: N-(5-(6-(3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide hydrochloride

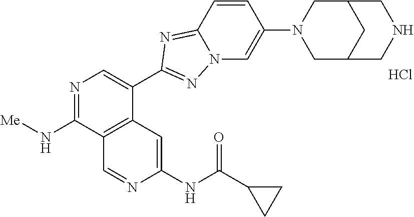 [SMILES: CNc1ncc(-c2nc3ccc(N4CC5CNCC(C5)C4)cn3n2)c2cc(NC(=O)C3CC3)ncc12 ; Cl]

A solution of tert-butyl 7-(2-(6-(cyclopropanecarboxamido)-1-(methylamino)-2,7-naphthyridin-4-yl)-[1,2,4]triazolo[1,5-a]pyridin-6-yl)-3,7-diazabicyclo[3.3.1]nonane-3-carboxylate (90.0 mg; 0.154 mmol; 1.00 eq.) in MeOH (1 mL) was added HCl (gas) (4 M in 1,4-dioxane, 4 mL). The resulting solution was stirred at room temperature for 1 hour. The desired product was detected via LCMS. The solvent was concentrated under vacuum to afford N-(5-(6-(3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide hydrochloride as a yellow solid (90.0 mg, crude). The crude product was used for the next step directly without further purification. LCMS (ESI) m/z 484.2, [M+H]⁺.

### Step 4: N-(5-(6-(7-methyl-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide

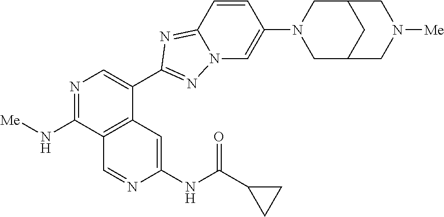 [SMILES: CNc1ncc(-c2nc3ccc(N4CC5CC(CN(C)C5)C4)cn3n2)c2cc(NC(=O)C3CC3)ncc12]

A solution of N-(5-(6-(3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide hydrochloride (90.0 mg; 0.186 mmol; 1.00 eq.), HCHO (33% w/w in water, 50.7 mg; 3.00 eq.) and DIPEA (240.5 mg; 1.861 mmol; 10.00 eq.) in CH₂Cl₂ (10 mL) was stirred at room temperature for 2 hours. To the above mixture was added NaBH(OAc)₃ (118.3 mg; 0.558 mmol; 3.00 eq.). The resulting solution was stirred at room temperature overnight. The desired product was detected via LCMS. The solvent was concentrated under vacuum. The residue was purified by flash chromatography, on silica gel column, using 0-15% of MeOH in CH₂Cl₂ (0.1% Et N) as eluent to provide the crude product. The crude product was slurried in MeOH (3 mL) at room temperature for 3 hours to precipitate solids. The solids were collected by filtration to provide N-(5-(6-(7-methyl-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide as an off-white solid (20.7 mg, 21.8%). LCMS (ESI) m/z 498.3, [M+H]⁺. ¹H NMR (400 MHz, DMSO-d₆) δ 10.91 (s, 1H), 9.44 (s, 1H), 9.39 (s, 1H), 8.73 (s, 1H), 8.23-8.18 (m, 1H), 8.03 (d, J=2.0 Hz, 1H), 7.67 (d, J=9.6 Hz, 1H), 7.58 (dd, J=9.6, 2.0 Hz, 1H), 3.69-3.60 (m, 2H), 3.22-3.09 (m, 2H), 3.08 (d, J=4.4 Hz, 3H), 2.91-2.85 (m, 2H), 2.20-2.08 (m, 8H), 1.72-1.66 (m, 1H), 1.60-1.54 (m, 1H), 0.89-0.80 (m, 4H).

### Example 503: Synthesis of 1V-(5-(6-methyl-[1,2,4]triazolo[1,5-a]pyridin-2-yl)-8-(methylamino)-2,7-naphthyridin-3-yl)cyclopropanecarboxamide

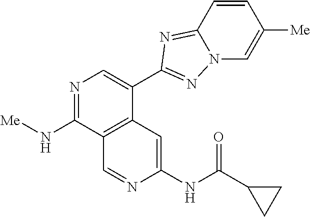 [SMILES: CNc1ncc(-c2nc3ccc(C)cn3n2)c2cc(NC(=O)C3CC3)ncc12]

### Step 1: ethyl N-[(5-methylpyridin-2-yl)carbamothioyl]carbamate

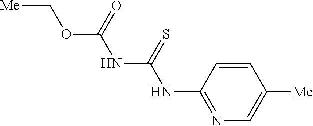 [SMILES: CCOC(=O)NC(=S)Nc1ccc(C)cn1]

A mixture of 5-methylpyridin-2-amine (5.00 g; 46.201 mmol; 1.00 eq.) and O-ethyl carbonisothiocyanatidate (6.65 g; 50.860 mmol; 1.10 eq.) in 1,4-dioxane (20 mL) was stirred at room temperature overnight. The desired product was detected via LCMS. The resulting mixture was concentrated under reduced pressure to afford ethyl N-[(5-methylpyridin-2-yl)carbamothioyl]carbamate as a orange solid (10.0 g, crude). LCMS (ESI) m/z 240.1, [M+H]⁺.

### Step 2: 6-methyl-[1,2,4]triazolo[1,5-a]pyridin-2-amine

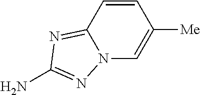 [SMILES: Cc1ccc2nc(N)nn2c1]

To a solution of ethyl N-[(5-methylpyridin-2-yl)carbamothioyl]carbamate (8.70 g; 36.221 mmol; 1.00 eq.) in a mixture solvent of MeOH/EtOH (1:1, 100 mL) were added NH₂OH·HCl (5.70 g; 54.331 mmol; 1.50 eq.) and DIPEA (10.50 g; 54.331 mmol; 1.50 eq.) dropwise at room temperature. The resulting mixture was stirred at 70° C. for 2 hours. LCMS indicated that starting material was consumed. The mixture was allowed to cool down to room temperature. The resulting mixture was concentrated under reduced pressure. The residue was purified by flash chromatography, on silica gel column, using 5-8% of MeOH in CH₂Cl₂ as eluent to provide 6-methyl-[1,2,4]triazolo[1,5-a]pyridin-2-amine as a white solid (5.00 g, 93.2%). LCMS (ESI) m/z 1.491, [M+H]⁺.

### Step 3: 2-chloro-6-methyl-[1,2,4]triazolo[1,5-a]pyridine

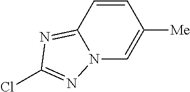 [SMILES: Cc1ccc2nc(Cl)nn2c1]

To a mixture of 6-methyl-[1,2,4]triazolo[1,5-a]pyridin-2-amine (5.00 g; 33.745 mmol; 1.00 eq.) in HCl (12 M in water, 12 mL) were added CuCl₂ (1.36 g; 10.123 mmol; 0.30 eq.) and a solution of NaNO₂ (4.66 g; 67.490 mmol; 2.00 eq.) in water (4 mL) dropwise at 0° C. The mixture was stirred at room temperature for 1 hour. The desired product was detected via LCMS. The reaction was quenched by the addition of a saturated NaHCO₃ aqueous solution (50 mL) and extracted with EtOAc (3×50 mL). The combined organic layers were washed with brine (3×30 mL), dried over anhydrous Na₂SO₄. The organic layer was concentrated under reduced pressure to afford 2-chloro-6-methyl-[1,2,4]triazolo[1,5-a]pyridine as an off-white solid (2.60 g, 46.0%). LCMS (ESI) m/z 168.0, [M+H]⁺.

### Step 4: N-(5-(6-methyl-[1,2,4]triazolo[1,5-a]pyridin-2-yl)-8-(methylamino)-2,7-naphthyridin-3-yl)cyclapropanecarboxamide

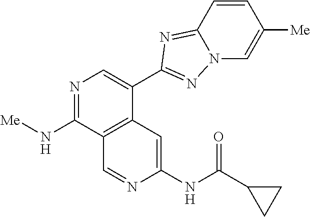 [SMILES: CNc1ncc(-c2nc3ccc(C)cn3n2)c2cc(NC(=O)C3CC3)ncc12]

N-(5-(6-methyl-[1,2,4]triazolo[1,5-a]pyridin-2-yl)-8-(methylamino)-2,7-naphthyridin-3-yl)cyclopropanecarboxamide was synthesized using a similar procedure that was previously described in Example 470, by using N-(8-(methylamino)-5-(4,4,5,5-tetramethyl-1,3,2-dioxaborolan-2-yl)-2,7-naphthyridin-3-yl)cyclopropanecarboxamide and 2-chloro-6-methyl-[1,2,4]triazolo[1,5-a]pyridine as the starting material. LCMS (ESI) m/z 374.2, [M+H]⁺. ¹H NMR (400 MHz, DMSO-d₆) δ 1.0.93 (s, 1H), 9.49 (s, 1H), 9.40 (s, 1H), 8.79 (s, 1H), 8.78 (d, J=2.0 Hz, 1H), 8.31-8.23 (m, 1H), 7.76 (d, J=9.6 Hz, 1H), 7.55 (dd, J=9.6, 2.0 Hz, 1H), 3.06 (d, J=4.4 Hz, 3H), 2.42 (s, 3H), 2.14-2.03 (m, 1H), 0.90-0.79 (m, 4H).

### Example 504: Synthesis of N-(5-(6-(⁷-methyl-9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide

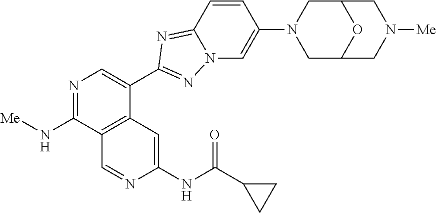 [SMILES: CNc1ncc(-c2nc3ccc(N4CC5CN(C)CC(C4)O5)cn3n2)c2cc(NC(=O)C3CC3)ncc12]

### Step 1: tert-butyl 7-(2-chloro-[1,2,4]triazolo[1,5-a]pyridin-6-yl)-9-oxa-3,7-diazabicyclo[3.3.1]nonane-3-carboxylate

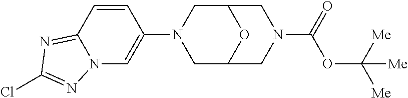 [SMILES: CC(C)(C)OC(=O)N1CC2CN(c3ccc4nc(Cl)nn4c3)CC(C1)O2]

A mixture of 6-bromo-2-chloro-[1,2,4]triazolo[1,5-a]pyridine (500.0 mg; 2,151 mmol; 1.00 eq.), tert-butyl 9-oxa-3,7-diazabicyclo[3.3.1]nonane-3-carboxylate (491.0 mg; 2.151 mmol; 1.00 eq.), Pd-PEPPSI-IHeptCl 3-chloropyridine (209.5 mg; 0.215 mmol; 0.10 eq.) and Cs₂CO₃ (2.80 g; 8.603 mmol, 4.00 eq.) in 1,4-dioxane (10 mL) was stirred at 100° C. overnight under nitrogen atmosphere. After the reaction was completed, the reaction was allowed to cool to room temperature and concentrated under reduced pressure. The residue was purified by flash chromatography, on silica gel column, using 20-40% of EtOAc in petroleum ether as eluent to provide tert-butyl 7-(2-chloro-[1,2,4]triazolo[1,5-a]pyridin-6-yl)-9-oxa-3,7-diazabicyclo[3.3.1]nonane-3-carboxylate as a white solid (400.0 mg, 47.9%). LCMS (ESI) m/z 380.1, [M+H]⁺.

### Step 2: tert-butyl 7-(2-(6-(cyclopropanecarboxamido)-1-(methlamino)-2,7-naphthyridin-4-yl)-[1,2,4]triazolo[1,5-a]pyridin-6-yl)-9-oxa-3,7-diazabicyclo[3.3.1]nonane-3-carboxylate

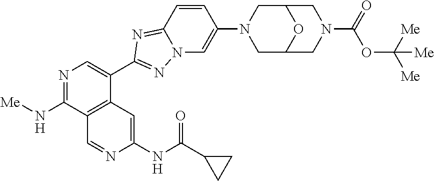 [SMILES: CNc1ncc(-c2nc3ccc(N4CC5CN(C(=O)OC(C)(C)C)CC(C4)O5)cn3n2)c2cc(NC(=O)C3CC3)ncc12]

A mixture of tert-butyl 7-(2-chloro-[1,2,4]triazolo[1,5-a]pyridin-6-yl)-9-oxa-3,7-diazabicyclo[3.3.1]nonane-3-carboxylate (500.0 mg; 1.316 mmol; 1.00 eq.), N-(8-(methylamino)-5-(4,4,5,5-tetramethyl-1,3,2-dioxaborolan-2-yl)-2,7-naphthyridin-3-yl)cyclopropanecarboxamide (484.7 mg; 1.316 mmol; 1.00 eq.), XPhos Pd G₃ (334.3 mg; 0.395 mmol; 0.30 eq.), XPhos (188.3 mg; 0.395 mmol; 0.30 eq.) and K₃PO₄ (838.2 mg; 3.949 mmol; 3.00 eq.) in a mixture solvent of 1,4-di oxane/water (5:1, 12 mL) was stirred at 110° C. for 1 hour under nitrogen atmosphere. The desired product was detected via LCMS. The resulting mixture was concentrated under reduced pressure. The residue was purified by flash chromatography on silica gel column using 5-10% of MeOH in CH₂Cl₂ as eluent to afford tert-butyl 7-(2-(6-(cyclopropanecarboxamido)-1-(methylamino)-2,7-naphthyridin-4-yl)-[1,2,4]triazolo[1,5-a]pyridin-6-yl)-9-oxa-3,7-diazabicyclo[3.3.1]nonane-3-carboxylate as a yellow solid (420.0 mg, 52.9%). LCMS (ESI) m/z 586.3, [M+H]⁺.

### Step 3: N-(5-(6-(9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide 2,2,2-trifluoroacetate

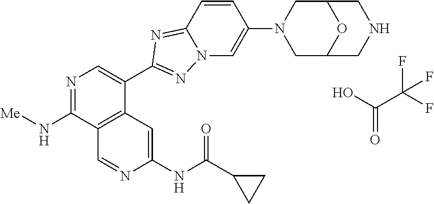

To a solution of ten-butyl 7-(2-(6-(cyclopropanecarboxamido)-1-(methylamino)-2,7-naphthyridin-4-yl)-[1,2,4]triazolo[1,5-a]pyridin-6-yl)-9-oxa-3,7-diazabicyclo[3.3.1]nonane-3-carboxylate (1.00 g; 1.707 mmol; 1.00 eq.) in CH₂Cl₂ (50 mL) was added trifluoroacetic acid (10 mL). The mixture was stirred at room temperature for 0.5 hour under nitrogen atmosphere. The desired product was detected via LCMS. The resulting mixture was concentrated under reduced pressure to afford N-(5-(6-(9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide 2,2,2-trifluoroacetate as a yellow solid (834.0 mg, crude). LCMS (ESI) m/z 486.2, [M+H]⁺.

### Step 4: N-(5-(6-(7-methyl-9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide

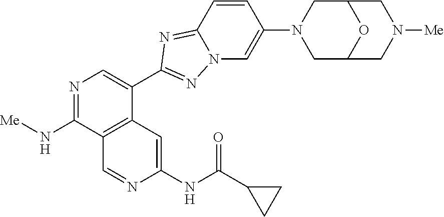 [SMILES: CNc1ncc(-c2nc3ccc(N4CC5CN(C)CC(C4)O5)cn3n2)c2cc(NC(=O)C3CC3)ncc12]

To a mixture of N-(5-(6-(9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide 2,2,2-trifluoroacetate (400.0 mg; 0.824 mmol; 1.00 eq.) and DIPEA (106.5 mg; 0.824 mmol; 1.00 eq.) in CH₂Cl₂ (10 mL) was added HCHO (35% w/w in water, 74.2 mg; 2.471 mmol; 3.00 eq.). The mixture was stirred at room temperature for 2 hours. To the above mixture was added NaBH(AcO)₃ (523.8 mg; 2.471 mmol; 3.00 eq.), the reaction was stirred at room temperature for another 1.5 hours. The desired product was detected via LCMS. The resulting mixture was concentrated under reduced pressure. The residue was purified by flash chromatography on silica gel column using 5-10% of MeOH in CH₂Cl₂ (0.1% Et₃N) as eluent to afford the crude product. The crude product was purified by recrystallization with a mixture solvent of CH₂Cl₂/MeOH (7:1, 30 mL) to 1.0 provide N-(5-(6-(7-methyl-9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide as a white solid (275.6 mg, 65.1%). LCMS (ESI) m/z 500.2, [M+H]⁺. ¹H NMR (400 MHz, DMSO-d₆) δ 10.92 (s, 1H), 9.44 (s, 1H), 9.40 (s, 1H), 8.74 (s, 1H), 8.26-8.18 (m, 1H), 8.14 (d, J=2.0 Hz, 1H), 7.72 (d, J=9.6 Hz, 1H), 7.63 (dd, J=9.6, 2.0 Hz, 1H), 4.11-4.02 (m, 2H), 3.70-3.63 (m, 2H), 3.15-3.06 (m, 2H), 3.05 (d, J=4.4 Hz, 3H), 2.92-2.84 (m, 2H), 2.36-2.29 (m, 2H), 2.11-2.01 (m, 4H), 0.89-0.81 (m, 4H).

### Example 505: Synthesis of N-(5-(8-fluoro-6-methyl-[1,2,4]triazolo[1,5-a]pyridin-2-yl)-8-(methylamino)-2,7-naphthyridin-3-yl)cyclopropanecarboxamide

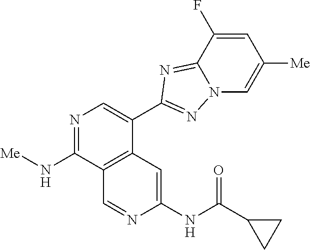 [SMILES: CNc1ncc(-c2nc3c(F)cc(C)cn3n2)c2cc(NC(=O)C3CC3)ncc12]

### Step 1: ethyl N-[(3-fluoro-5-methylpyridin-2-yl)carbamothioyl]carbamate

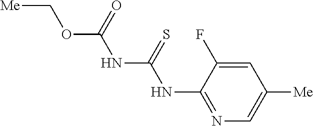 [SMILES: CCOC(=O)NC(=S)Nc1ncc(C)cc1F]

A solution of 3-fluoro-5-methylpyridin-2-amine (200.3 mg; 1.582 mmol; 1.00 eq.) and O-ethyl carbonisothiocyanatidate (228.2 mg; 1.734 mmol; 1.10 eq.) in 1,4-dioxane (3 mL) was stirred at room temperature overnight under nitrogen atmosphere. The desired product was detected via LCMS. The resulting mixture was concentrated under reduced pressure to afford ethyl N-[(3-fluoro-5-methylpyridin-2-yl)carbamothioyl]carbamate as a yellow solid (400.1 mg, 98.0%). LCMS (ESI) m/z 258.1, [M+H]⁺.

### Step 2: 8-fluoro-6-methyl-[1,2,4]triazolo[1,5-a]pyridin-2-amine

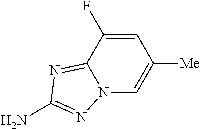 [SMILES: Cc1cc(F)c2nc(N)nn2c1]

To a stirred solution of ethyl N-[(3-fluoro-5-methylpyridin-2-yl)carbamothioyl]carbamate (380.2 mg; 1.472 mmol; 1.00 eq.) and hydroxylamine hydrochloride (1.519 mg; 2.184 mmol; 1.50 eq.) in a mixture solvent of MeOH/EtOH (1:1, 5 mL) was added DIPEA (284.4 mg; 2.202 mmol; 1.50 eq.). The resulting mixture was stirred at 70° C. for 2 hours. The desired product was detected via LCMS. The precipitated solids were collected by filtration and washed with MeOH (1 mL) to afford 8-fluoro-6-methyl-[1,2,4]triazolo[1,5-a]pyridin-2-amine as a white solid (170.2 mg, 69.2%). LCMS (ESI) m/z 167.1, [M+H]⁺.

### Step 3: 2-chloro-8-fluoro-6-methyl-[1,2,4]triazolo[1,5-a]pyridine

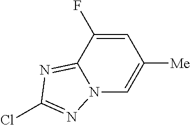 [SMILES: Cc1cc(F)c2nc(Cl)nn2c1]

To a stirred solution of 8-fluoro-6-methyl-[1,2,4]triazolo[1,5-a]pyridin-2-amine (170.2 mg; 1.081 mmol; 1.00 eq.) and CuCl₂ (32.1 mg; 0.231 mmol; 0.20 eq.) in HCl (1.2 M in water, 4 mL) was added a solution of NaNO₂ (149.3 mg; 2.164 mmol; 2.00 eq.) in water (1 mL) dropwise at 0° C. The mixture was stirred at room temperature for 1 hour. The desired product was detected via LCMS. The reaction was diluted with water (20 mL) and neutralized to pH=7 with a saturated Na₂CO₃ solution. The resulting mixture was extracted with EtOAc (3×20 mL). The combined organic layers were washed with brine (3×1.0 mL) and dried over anhydrous Na₂SO₄, filtered, and concentrated under reduced pressure to afford 2-chloro-8-fluoro-6-methyl-[1,2,4]triazolo[1,5-a]pyridine as a white solid (160.1 mg, 79.5%). LCMS (ESI) m/z 186.0, [M+H]⁺.

### Step 4: N-(5-(8-fluoro-6-methyl-[1,2,4]triazolo[1,5-a]pyridin-2-yl)-8-(methylanilino)-2,7-naphthyridin-3-yl)cyclopropanecarboxamide

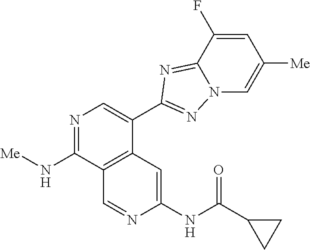 [SMILES: CNc1ncc(-c2nc3c(F)cc(C)cn3n2)c2cc(NC(=O)C3CC3)ncc12]

N-(5-(8-fluoro-6-methyl-[1,2,4]triazolo[1,5-a]pyridin-2-yl)-8-(methylamino)-2,7-naphthyridin-3-yl)cyclopropanecarboxamide was synthesized using a similar procedure that was previously described in Example 470, by using N-(8-(methylamino)-5-(4,4,5,5-tetramethyl-1,3,2-dioxaborolan-2-yl)-2,7-naphthyridin-3-yl)cyclopropanecarboxamide and 2-chloro-8-fluoro-6-methyl-[1,2,4]triazolo[1,5-a]pyridine as the starting material. LCMS (ESI) m/z 392.2, [M+H]⁺. ¹H NMR (400 MHz, DMSO-d₆) δ 10.95 (s, 1H), 9.47 (s, 1H), 9.42 (s, 1H), 8.82 (s, 1H), 8.72-8.70 On, 1H), 8.36-8.30 (m, 1H), 7.59-7.53 (m, 1H), 3.06 (d, J=4.4 Hz, 3H), 2.41 (s, 3H), 2.12-2.04 (m, 1H), 0.90-0.80 (m, 4H).

### Example 506: Synthesis of (Z)—N-(5-(5-(2-ethoxyvinyl)-[1,2,4]triazolo[1,5-a]pyridin-2-yl)-8-(methylamino)-2,7-naphthyridin-3-yl)cyclopropanecarboxamide

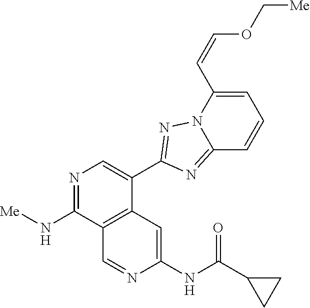 [SMILES: CCO/C=C\c1cccc2nc(-c3cnc(NC)c4cnc(NC(=O)C5CC5)cc34)nn12]

### Step 1: 5-bronco-2-chloro-[1,2,4]triazolo[1,5-a]pyridine

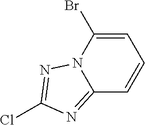 [SMILES: Clc1nc2cccc(Br)n2n1]

To a stirred mixture of 5-bromo-[1,2,4]triazolo[1,5-a]pyridin-2-amine (2.00 g; 9.388 mmol; 1 eq.) and CuCl₂ (329.5 mg; 2.451 mmol; 0.26 eq.) in hydrochloric acid (12 M in water, 20.9 mL) was added a solution of NaNO₂ (777.0 mg; 11.262 mmol; 1.20 eq.) in water (4.3 mL) dropwise at 5° C. The mixture was stirred at 5° C. for 30 minutes and at room temperature for 18 hours. The desired product was detected via LCMS. The yellow mixture was diluted with water. (100 mL) and precipitates were formed. The solids were collected by filtration, washed with water and dried under pressure to afford 5-bromo-2-chloro-[1,2,4]triazolo[1,5-a]pyridine as a off-white solid (1.8 g, 82.4%). LCMS (ESI) m/z 231.9, [M+H]⁺.

### Step 2: (Z)-2-chloro-5-(2-ethoxyvinyl)-[1,2,4]triazolo[1,5-a]pyridine

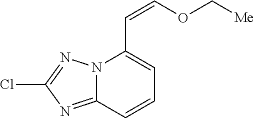 [SMILES: CCO/C=C\c1cccc2nc(Cl)nn12]

To a stirring mixture of 5-bromo-2-chloro-[1,2,4]triazolo[1,5-a]pyridine (210.0 mg; 0.909 mmol; 1.00 eq.) in 1,4-dioxane (1.0 mL) was added (Z)-tributyl(2-ethoxyvinyl)stannane (658.1 mg; 1.818 mmol; 2.00 eq.), Pd(PPh₃)₂Cl₂ (127.6 mg; 0.181 mmol; 0.20 eq.) and CuI (25.9 mg; 0.136 mmol; 0.15 eq.). The mixture was stirred at 90° C. for 1 hour under nitrogen atmosphere. The desired product was detected via LCMS. The reaction was concentrated under pressure. The residue was purified by flash chromatography on silica gel column using 0-20% of EtOAc in petroleum ether as eluent to provide (Z)-2-chloro-5-(2-ethoxyvinyl)-[1,2,4]triazolo[1,5-a]pyridine as a light yellow solid (135.0 mg, 66.6%). LCMS (ESI) m/z 224.1, [M+H]⁺.

### Step 3: (Z)—N-(5-(5-(2-ethoxyvinyl)-[1,2,4]triazolo[1,5-a]pyridin-2-yl)-8-(methylamino)-2,7-naphthyridin-3-yl)cydopropanecarboxamide

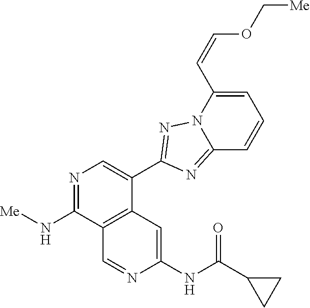 [SMILES: CCO/C=C\c1cccc2nc(-c3cnc(NC)c4cnc(NC(=O)C5CC5)cc34)nn12]

(Z)—N-(5-(5-(2-ethoxyvinyl)-[1,2,4]triazolo[1,5-a]pyridin-2-yl)-8-(methylamino)-2,7-naphthyridin-3-yl)cyclopropanecarboxamide was synthesized using a similar procedure that was previously described in Example 470, by using N-(8-(methylamino)-5-(4,4,5,5-tetramethyl-1,3,2-dioxaborolan-2-yl)-2,7-naphthyridin-3-yl)cyclopropanecarboxamide and (Z)-2-chloro-5-(2-ethoxyvinyl)-[1,2,4]triazolo[1,5-a]pyridine as the starting material. LCMS (ESI) m/z 430.2, [M+H]⁺. ¹H NMR (400 MHz, DMSO-d₆) δ 10.97 (s, 1H), 9.94 (s, 1H), 9.41 (s, 1H), 8.95 (s, 1H), 8.32-8.27 (m, 1H), 7.68-7.55 (m, 3H), 6.97 (d, J 7.2 Hz, 1H), 6.61 (d, J=7.2 Hz, 1H), 4.24 (q, 0.1=7.2 Hz, 2H), 3.06 (d, J=4.4 Hz, 3H), 2.15-2.04 (m, 1H), 1.37 (d, J=7.2 Hz, 3H), 0.96-0.81 (m, 4H).

### Example 507: Synthesis of N-(5-(5-(2-ethoxyethyl)-[1,2,4]triazolo[1,5-a]pyridin-2-yl)-8-(methylamino)-2,7-naphthyridin-3-yl)cyclopropanecarboxamide

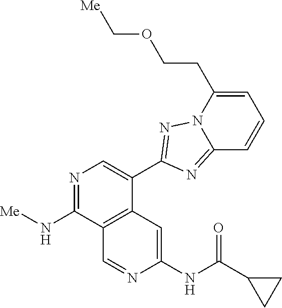 [SMILES: CCOCCc1cccc2nc(-c3cnc(NC)c4cnc(NC(=O)C5CC5)cc34)nn12]

A solution of (Z)—N-(5-(5-(2-ethoxyvinyl)-[1,2,4]triazolo[1,5-a]pyridin-2-yl)-8-(methylamino)-2,7-naphthyridin-3-yl)cyclopropanecarboxamide (Example 506) (52.0 mg; 0.121 mmol; 1.00 eq.) in MeOH (3 mL) was added 10% Pd/C (52.0 mg; 100% w/w) under nitrogen atmosphere. The mixture was hydrogenated at room temperature overnight under hydrogen atmosphere (2 atm). LCMS indicated that the starting material was consumed. The resulting mixture was filtered, the filtrate was concentrated under reduced pressure. The residue was purified by flash chromatography on pre-packed C18 column using 10-46% of MeOH in water (10 mmol/L NH₄HCO₃) as eluent to provide N-(5-(5-(2-ethoxyethyl)-[1,2,4]triazolo[1,5-a]pyridin-2-yl)-8-(methylamino)-2,7-naphthyridin-3-yl)cyclopropanecarboxamide as an off-white solid (12.8 mg, 23.3%). LCMS (ESI) m/z 432.2, [M+H]⁺. ¹H NMR (400 MHz, DMSO-d₆) δ 1.0.96 (s, 1H), 9.77 (s, 1H), 9.41 (s, 1H), 8.91 (s, 1H), 8.33-8.26 (m, 1H), 7.73 (d, J=8.8 Hz, 1H), 7.63 (dd, J=8.8, 6.8 Hz, 1H), 7.08 (d, J=6.8 Hz, UT), 3.94 (t, J=6.4 Hz, 2H), 3.54-3.44 (m, 4H), 3.07 (d, 4.4 Hz, 3H), 2.14-2.03 (m, 1H), 1.07 (t, J=6.8 Hz, 3H), 0.91-0.79 (m, 41-1).

### Example 508: Synthesis of (S)—N-(5-(5-fluoro-6-(2-methylmorpholino)-[1,2,4]triazolo[1,5-a]pyridin-2-yl)-8-(methylamino)-2,7-naphthyridin-3-yl)cyclopropanecarboxamide

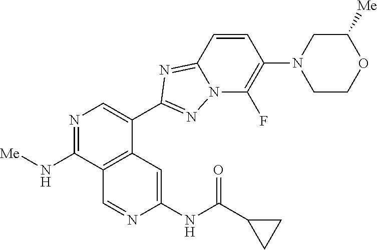 [SMILES: CNc1ncc(-c2nc3ccc(N4CCO[C@@H](C)C4)c(F)n3n2)c2cc(NC(=O)C3CC3)ncc12]

A solution of (S)—N-(8-(methylamino)-5-(6-(2-methylmorpholino)-[1,2,4]triazolo[1,5-a]pyridin-2-yl)-2,7-naphthyridin-3-yl)cyclopropanecarboxamide (Example 478) (35.0 mg; 0.076 mmol; 1.00 eq.) and Selectfluor (35.2 mg; 0.099 mmol; 1.30 eq.) in MeCN (5 mL) was stirred at room temperature for 1.5 hours under nitrogen atmosphere. The desired product was detected via LCMS. The reaction was quenched with water (10 mL) at room temperature. The resulting mixture was extracted with EtOAc (3×15 mL). The combined organic layers were washed with brine (2×5 mL), dried over anhydrous Na₂SO₄, filtered, and concentrated under reduced pressure. The residue was purified by flash chromatography on silica gel column using 0-10% of MeOH in CH₂Cl₂ as eluent to provide (S)—N-(5-(5-fluoro-6-(2-methylmorpholino)-[1,2,4]triazolo[1, 5-a]pyridin-2-yl)-8-(methylamino)-2,7-naphthyridin-3-yl)cyclopropanecarboxamide as a pink solid (6.4 mg, 17.1%). LCMS (ESI) m/z 477.2, [M+H]⁺. ¹H NMR (400 MHz, DMSO-d₆) δ 10.93 (s, 1H), 9.53 (s, 1H), 9.41 (s, 1H), 8.83 (s, 1H), 8.34-8.30 (m, 1H), 7.85-7.71 (m, 1H), 7.69 (d, J=9.6 Hz, 1H), 3.95-3.88 (m, 1H), 3.77-3.68 (m, 2H), 3.25-3.10 (m, 2H), 3.06 (d, J=4.4 Hz, 3H), 3.04-2.95 (m, 1H), 2.72-2.65 (m, 1H), 2.10-2.01 (m, 1H), 1.15 (d, J=6.0 Hz, 3H), 0.90-0.80 (m, 4H).

### Example 509: Synthesis of (S)—N-(5-(5-chloro-6-(2-methylmorpholino)-[1,2,4]triazolo[1,5-a]pyridin-2-yl)-8-(methylamino)-2,7-naphthyridin-3-yl)cyclopropanecarboxamide

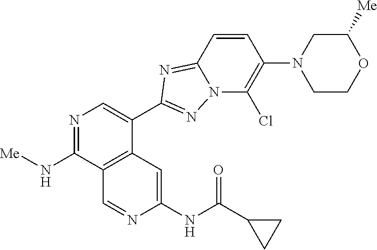 [SMILES: CNc1ncc(-c2nc3ccc(N4CCO[C@@H](C)C4)c(Cl)n3n2)c2cc(NC(=O)C3CC3)ncc12]

A solution of (S)—N-(8-(methylamino)-5-(6-(2-methylmorpholino)-[1,2,4]triazolo[1,5-a]pyridin-2-yl)-2,7-naphthyridin-3-yl)cyclopropanecarboxamide (Example 478) (76.0 mg; 0.166 mmol; 1.00 eq.) and NCS (22.1 mg; 0.166 mmol; 1.00 eq.) in DMF (4 mL) was stirred at 60° C. for 1.5 hours under nitrogen atmosphere. The desired product was detected via LCMS. The mixture was purified by flash chromatography on pre-packed C18 column using 60-80% of MeOH in water (10 mmol/L NH₄HCO₃) as eluent to provide (S)—N-(5-(5-chloro-6-(2-methylmorpholino)-[1,2,4]triazolo[1, 5-a]pyridin-2-yl)-8-(methylamino)-2,7-naphthyridin-3-yl)cyclopropanecarboxamide as a white solid (56.1 mg, 68.1%). LCMS (ESI) m/z 493.2, [M+H]⁺. ¹H NMR (400 MHz, DMSO-d₆) δ 10.92 (s, 1H), 9.57 (s, 1H), 9.41 (s, 1H), 8.82 (s, 1H), 8.34-8.30 (m, 1H), 7.89-7.76 (m, 2H), 3.96-3.89 (m, 1H), 3.80-3.68 (m, 2H), 3.16-3.09 (m, 2H), 3.06 (d, J=4.4 Hz, 3H), 3.01-2.90 (m, 1H), 2.72-2.63 (m, 1H), 2.13-2.02 (m, 1H), 1.16 (d, J=6.0 Hz, 3H), 0.90-0.78 (m, 4H).

### Example 510: Synthesis of N-(8-(methylamino)-5-(6-(2-(methylsulfonyl)ethoxy)-[1,2,4]triazolo[1,5-a]pyridin-2-yl)-2,7-naphthyridin-3-yl)cyclopropanecarboxamide

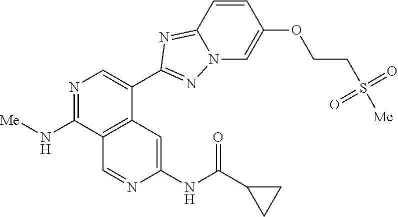 [SMILES: CNc1ncc(-c2nc3ccc(OCCS(C)(=O)=O)cn3n2)c2cc(NC(=O)C3CC3)ncc12]

### Step 1: N-(5-(6-hydroxy-[1,2,4]triazolo[1,5-a]pyridin-2-yl)-8-(methylamino)-2,7-naphthyridin-3-yl)cyclopropanecarboxamide

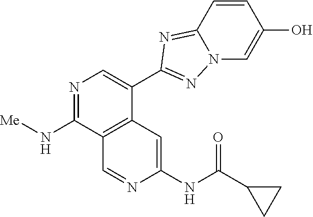 [SMILES: CNc1ncc(-c2nc3ccc(O)cn3n2)c2cc(NC(=O)C3CC3)ncc12]

To a solution of N-(5-(6-(benzyloxy)-[1,2,4]triazolo[1,5-a]pyridin-2-yl)-8-(methylamino)-2,7-naphthyridin-3-yl)cyclopropanecarboxamide (Example 500) (200.0 mg; 0.430 mmol; 1.00 eq.) in THF (10 mL) was added 10% Pd/C (200.0 mg; 100% w/w) under nitrogen atmosphere. The mixture was hydrogenated at 40° C. for 1 hour under hydrogen atmosphere (2 atm). The desired product was detected via LCMS. The resulting mixture was filtered, the filter cake was washed with THF (5 mL). The filtrate was concentrated under reduced pressure to afford N-(5-(6-hydroxy-[1,2,4]triazolo[1,5,a]pyridin-2-yl)-8-(methylamino)-2,7-naphthyridin-3-yl)cyclopropanecarboxamide as a light brown solid (130.0 mg, 80.7%). LCMS (ESI) m/z 376.1, [M+H]⁺.

### Step 2: N-(8-(methylamino)-5-(6-(2-(methylthio)ethoxy)-[1,2,4]triazolo[1,5-a]pyridin-2-yl)-2,7-naplathyridin-3-yl)cyclopropanecarboxamide

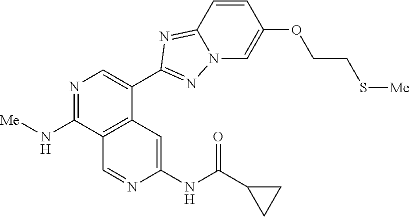 [SMILES: CNc1ncc(-c2nc3ccc(OCCSC)cn3n2)c2cc(NC(=O)C3CC3)ncc12]

To a solution of (2-chloroethyl)(methyl)sulfane (38.9 mg; 0.352 mmol; 1.10 eq.) and N-(5-(6-hydroxy-[1,2,4]triazolo[1,5-a]pyridin-2-yl)-8-(methylamino)-2,7-naphthyridin-3-yl)cyclopropanecarboxamide (120.0 mg; 0.320 mmol; 1.00 eq.) in DMF (2 mL) was added Cs₂CO₃ (208.0 mg; 0.640 mmol; 2.00 eq.). The mixture was stirred at 100° C. for 2 hours under nitrogen atmosphere. The desired product was detected via LCMS. The resulting mixture was diluted with water (10 mL) and extracted with EtOAc (3×10 mL). The combined organic layers were washed with brine (2×1.0 mL), dried over anhydrous Na₂SO₄, filtered, and concentrated under reduced pressure. The residue was purified by flash chromatography on silica gel column using 5-8% of MeOH in CH₂Cl₂ as eluent to provide N-(8-(methylamino)-5-(6-(2-(methylthio)ethoxy)-[1,2,4]triazolo[1,5-a]pyridin-2-yl)-2,7-naphthyridin-3-yl)cyclopropanecarboxamide as a white solid (55.0 mg, 38.3%). LCMS (ESI) m/z 450.2, [M+H]⁺.

### Step 3: N-(8-(methylamino)-5-(6-(2-(methylsulfonyl)ethoxy)-[1,2,4]triazolo[1,5-a]pyridin-2-yl)-2,7-naphthyridin-3-yl)cyclopropanecarboxamide

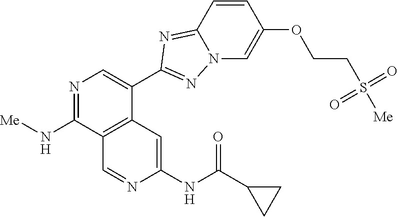 [SMILES: CNc1ncc(-c2nc3ccc(OCCS(C)(=O)=O)cn3n2)c2cc(NC(=O)C3CC3)ncc12]

A solution of N-(8-(methylamino)-5-(6-(2-(methyl thio)ethoxy)-[1,2,4]triazolo[1,5-a]pyridin-2-yl)-2,7-naphthyridin-3-yl)cyclopropanecarboxamide (50.0 mg; 0.111 mmol; 1.00 eq.) and m-CPBA (57.6 mg; 0.333 mmol; 3.00 eq.) in CH₂Cl₂ (3 mL) was stirred at room temperature for 2 hours under nitrogen atmosphere. The desired product was detected via LCMS. The reaction was quenched with a saturated Na₂SO₃ solution (20 mL) and extracted with CH₂Cl₂ (3×20 mL). The combined organic layers were washed with brine (3×10 mL), dried over anhydrous Na₂SO₄. After filtration, the filtrate was concentrated under reduced pressure. The residue was purified by flash chromatography on silica gel column using 20-30% of THF in CH₂Cl₂ as eluent to provide the crude product. The crude product was repurified by Prep-Achiral-SFC (Column: GreenSep Basic 3×15 cm, 5 μm; Mobile Phase A: CO₂, Mobile Phase B: MeOH (0.1% 2 M NH₃-MeOH); Flow rate: 75 mL/min; Gradient: isocratic 30% B) to provide N-(8-(methylamino)-5-(6-(2-(methylsulfonyl)ethoxy)-[1,2,4]triazolo[1,5-a]pyridin-2-yl)-2,7-naphthyridin-3-yl)cyclopropanecarboxamide as an off-white solid (8.1 mg, 15.1%). LCMS (ESI) m/z 482.2, [M+H]⁺. ¹H NMR (400 MHz, DMSO-d₆) δ 10.92 (s, 1H), 9.41 (s, 1H), 9.40 (s, 1H), 8.83 (d, J=2.0 Hz, 1H), 8.74 (s, 1H), 8.28-8.23 (m, 1H), 7.80 (d, J=9.6 Hz, 1H), 7.51. (dd, J=9.6, 2.0 Hz, 1H), 4.51 (t, J=5.6 Hz, 2H), 3.69 (t, J=5.6 Hz, 2H), 3.11 (s, 3H), 3.06 (d, J=4.4 Hz, 3H), 2.13-2.02 (m, 1H), 0.91-0.78 (m, 4H).

### Example 511: Synthesis of N-(5-(6-methoxy-[1,2,4]triazolo[1,5-a]pyridin-2-yl)-8-((methyl-d₃)amino)-2,7-naphthyridin-3-yl)cyclapropanecarboxamide

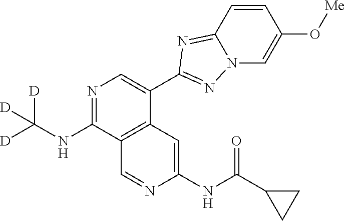 [SMILES: [2H]C([2H])([2H])Nc1ncc(-c2nc3ccc(OC)cn3n2)c2cc(NC(=O)C3CC3)ncc12]

### Step 1: 6-chloro-N-(methyl-d₃)-2,7-naphthyridin-1-amine

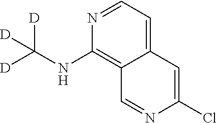 [SMILES: [2H]C([2H])([2H])Nc1nccc2cc(Cl)ncc12]

To a solution of 6-chloro-2,7-naphthyridin-1(2H)-one (10.00 g; 55.370 mmol; 1.00 eq.) and PyBOP (60.00 g; 115.290 mmol; 2.08 eq.) in DMA (200 mL) was added DIEA (60.00 g; 464.220 mmol; 8.38 eq.) at room temperature. The resulting solution was stirred at 40° C. for 3 hours under nitrogen atmosphere. To the above solution was added methan-d₃-amine hydrochloride (10.00 g; 41.750 mmol; 2.56 eq.). The resulting mixture was stirred at 40° C. for 1.6 hours. LCMS indicates that the starting material was consumed. The resulting mixture was diluted with brine (600 mL) and extracted with EtOAc (3×200 mL). The organic layers were washed with brine (3×200 mL), dried over anhydrous Na₂SO₄, filtered, and concentrated under reduced pressure. The residue was purified by flash chromatography on silica gel column using 2-6% of MeOH in CH₂Cl₂ as eluent to afford 6-chloro-N-(methyl-d₃)-2,7-naphthyridin-1-amine as a off-white solid (10.00 g, 91.8%). LCMS (ESI) m/z 197.1, [M+H]⁺.

### Step 2: N-(8-((methyl-d₃)amino)-2,7-naphthyridin-3-yl)cyclopropanecarboxamide

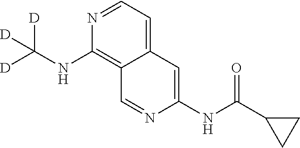 [SMILES: [2H]C([2H])([2H])Nc1nccc2cc(NC(=O)C3CC3)ncc12]

To a solution of 6-chloro N-(methyl-d₃)-2,7-naphthyridin-1-amine (10.00 g; 50.850 mmol; 1.00 eq.) and cyclopropanecarboxamide (6.50 g; 76.370 mmol; 1.50 eq.) in 1,4-dioxane (100 mL) were added Cs₂CO₃ (7.8 g; 102.45 mmol; 2.00 eq.) and Pd₂(dba)₃ (5.00 g; 5.460 mmol; 0.11 eq.) and XantPhos (6.00 g; 1.0.360 mmol; 0.20 eq.) at room temperature under nitrogen atmosphere. The resulting mixture was stirred at 110° C. for 16 hours. The desired product was detected via LCMS. The mixture was allowed to cool down to room temperature. The resulting mixture was diluted with EtOAc (500 mL). The resulting mixture was filtered, the filter cake was washed with EtOAc. The filtrate was concentrated under reduced pressure. The residue was purified by flash chromatography on silica gel column using 3-10% of MeOH in CH₂Cl₂ as eluent to afford N-(8-((methyl-d₃)amino)-2,7-naphthyridin-3-yl)cyclopropanecarboxamide as a white solid (11.00 g, 88.1%). LCMS (ESI) m/z 246.1, [M+H]⁺.

### Step 3: N-(5-bromo-8-((methyl-d₃)amino)-2,7-naphthyridin-3-yl)cyclopropanecarboxamide

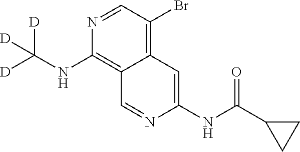 [SMILES: [2H]C([2H])([2H])Nc1ncc(Br)c2cc(NC(=O)C3CC3)ncc12]

A solution of N-(8-((methyl-d₃)amino)-2,7-naphthyridin-3-yl)cyclopropanecarboxamide (9.00 g; 36.690 mmol; 1.00 eq.) and NBS (5.22 g; 29.350 mmol; 0.80 eq.) in DMF (30 mL) was stirred at room temperature for 1 hour. The desired product was detected via LCMS. The resulting mixture was diluted with water (300 mL). The precipitated solids were collected by filtration and washed with water (2×30 mL). The solids were dried under reduced pressure o afford N-(5-bromo-8-((methyl-d₃)amino)-2,7-naphthyridin-3-yl)cyclopropanecarboxamide as a white solid (9.50 g, 79.8%). LCMS (ESI) m/z 324.0, [M+H]⁺.

Each intermediate in Table 19 below was prepared using a similar experimental procedure to prepare N-(5-bromo-8-((methyl-d₃)amino)-2,7-naphthyridin-3-yl)cyclopropanecarboxamide, where methan-d₃-amine hydrochloride was replaced with the reagent as shown Table 19 below.

TABLE 19

| Intermediate | Reagent | MS |
|---|---|---|
| 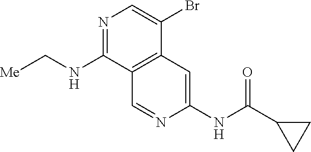 [SMILES: CCNc1ncc(Br)c2cc(NC(=O)C3CC3)ncc12] | ethanamine | LCMS (ESI) m/z 334.1, [M + H]⁺. |
| 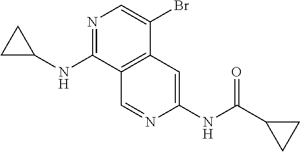 [SMILES: O=C(Nc1cc2c(Br)cnc(NC3CC3)c2cn1)C1CC1] | cyclopropanamine | LCMS (ESI) m/z 347.2, [M + H]⁺. |

### Step 4: N-(8-((methyl-d₃)amino)-5-(4,4,5,5-tetramethyl-1,3,2-dioxaborolan-2-yl)-2,7-naphthyridin-3-yl)cyclopropanecarboxamide

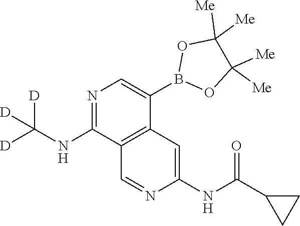 [SMILES: [2H]C([2H])([2H])Nc1ncc(B2OC(C)(C)C(C)(C)O2)c2cc(NC(=O)C3CC3)ncc12]

A mixture of N-(5-bromo-8-((methyl-d₃)amino)-2,7-naphthyridin-3-yl)cyclopropanecarboxamide (1.50 g; 4.627 mmol; 1.00 eq.), 4,4,4′,4′,5,5,5′,5′-octamethyl-2,2′-bi(1,3,2-dioxaborolane) (11.80 g; 46.468 mmol; 10.04 eq.), Pd(dppf)Cl₂·CH₂Cl₂ (379.0 mg; 0.465 mmol; 0.10 eq.) and KOAc (910.0 mg; 9.272 mmol; 2.00 eq.) in 1,4-dioxane (75 mL) was stirred at 100° C. for 2 hours under nitrogen atmosphere. The desired product was detected via LCMS. The resulting mixture was concentrated under reduced pressure. The residue was slurried in a mixture solvent of petroleum ether/EtOAc (10:1, 50 mL) to precipitate solids. The precipitated solids were collected by filtration and washed with a mixture solvent of petroleum ether/EtOAc(10:1, 20 mL). The solids were dried under reduced pressure to afford N-(8-((methyl-d₃)amino)-5-(4,4,5,5-tetramethyl-1,3,2-dioxaborolan-2-yl)-2,7-naphthyridin-3-yl)cyclopropanecarboxamide as a brown crude solid (2.6 g, crude). LCMS (ESI) m/z 372.2, [M+H]⁺.

### Step 5: N-(5-(6-methoxy-[1,2,4]triazolo[1,5-a]pyridin-2-yl)-8-((methyl-d₃)amino)-2,7-naphthyridin-3-yl)cyclopropanecarboxamide

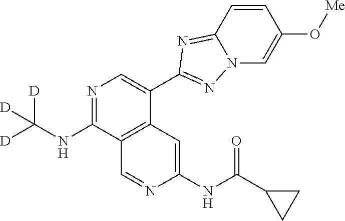 [SMILES: [2H]C([2H])([2H])Nc1ncc(-c2nc3ccc(OC)cn3n2)c2cc(NC(=O)C3CC3)ncc12]

A solution of 2-chloro-6-methoxy-[1,2,4]triazolo[1,5-a]pyridine (Example 470, Step 3) (200.0 mg; 1.089 mmol; 1.00 eq.) and N-(8-((methyl-d₃)amino)-5-(4,4,5,5-tetramethyl-1,3,2-dioxaborolan-2-yl)-2,7-naphthyridin-3-yl)cyclopropanecarboxamide (404.0 mg; 1.088 mmol; 1.00 eq.) in a mixture solvent of 1,4-dioxane/water (5:1, 9.6 mL). To the above solution were added XPhos Pd G₃ (276.0 mg; 0.326 mmol; 0.30 eq.), XPhos (155.0 mg; 0.325 mmol; 0.30 eq.) and K₃PO₄ (693.0 mg; 3.265 mmol; 3.00 eq.). The reaction was stirred at 110° C. for 1.5 hours under nitrogen atmosphere. The desired product was detected via LCMS. The resulting mixture was concentrated under reduced pressure. The residue was purified by flash chromatography on silica gel column using 1-9% of MeOH in CH₂Cl₂ as eluent to provide the product (312.0 mg, 72.9%). 50 mg of the product was further purified by flash chromatography on pre-packed C18 column using 5-50% of MeCN in water (10 mmol/L NH₄HCO₃) as eluent to provide N-(5-(6-methoxy-[1,2,4]triazolo[1,5-a]pyridin-2-yl)-8-((methyl-d₃)amino)-2,7-naphthyridin-3-yl)cyclopropanecarboxamide as a light yellow solid (25.2 mg). LCMS (ESI) m/z 393.2, [M+H]⁺. ¹H NMR (400 MHz, DMSO-d₆) δ 10.91 (s, 1H), 9.40 (s, 2H), 8.73 (s, 1H), 8.67 (d, J=2.0 Hz, 1H), 8.24-8.20 (m, 1H), 7.78 (d, J=9.6 Hz, 1H), 7.46 (dd, J=9.6, 2.0 Hz, 1H), 3.90 (s, 3H), 2.12-2.03 (m, 1H), 0.91-0.80 (m, 4H).

### Examples 512 and 513

Each compound in Table 4 below was prepared using a similar experimental procedure to prepare in Example 511, using N-(8-((methyl-d₃)amino)-5-(4,4,5,5-tetramethyl-1,3,2-dioxaborolan-2-yl)-2,7-naphthyridin-3-yl)cyclopropanecarboxamide as the common intermediate and appropriate 2-chloro-[1,2,4]triazolo[1,5-a]pyridine substrates.

| Example | Structure and Name | MS and ¹H NMR |
|---|---|---|
| Example 512 | 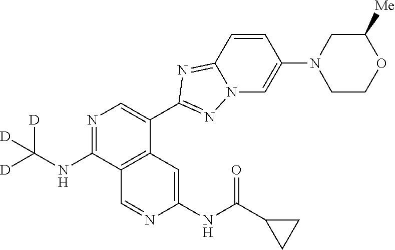 | LCMS (ESI) m/z 462.2 [M + H]⁺. ¹H NMR (400 MHz, DMSO-d₆) δ 10.93 (s, 1H), 9.40 (s, 1H), 9.39 (s, 1H), 8.71 (s, 1H), 8.33 (d, J = 2.0 Hz, 1H), 8.27- 8.18 (m, 1H), 7.74 (d, J = 9.6 Hz, 1H), 7.69 (d, J = 9.6, 2.0 Hz, 1H), 3.99- 3.91 (m, 1H), 3.78-3.60 (m, 3H), 3.54-3.47 (m, 1H), 2.79-2.69 (m, 1H), 2.46-2.36 (m, 1H), 2.11-2.02 (m, 1H), 1.18 (d, J = 6.0 Hz, 3H), 0.91- 0.81 (m, 4H). |
| | (R)-N-(8-((methyl-d₃)amino)-5- | |
| | (6-(2-methylmorpholino)- | |
| | [1,2,4]triazolo[1,5-a]pyridin-2- | |
| | yl)-2,7-naphthyridin-3- | |
| | yl)cyclopropanecarboxamide | |
| Example 513 | 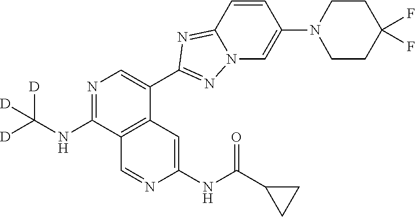 | LCMS (ESI) m/z 482.2, [M + H]⁺. ¹H NMR (400 MHz, DMSO-d₆) δ 10.92 (s, 1H), 9.40 (s, 1H), 9.38 (s, 1H), 8.70 (s, 1H), 8.45 (d, J = 2.0 Hz, 1H), 8.24- 8.18 (m, 1H), 7.74 (d, J = 9.6 Hz, 1H), 7.70 (dd, J = 9.6, 2.0 Hz, 1H), 3.36-3.32 (m, 4H), 2.19-2.05 (m, 5H), 0.89-0.80 (m, 4H). |
| | N-(5-(6-(4,4-difluoropiperidin-1- | |
| | yl)-[1,2,4]triazolo[1,5-a]pyridin- | |
| | 2-yl)-8-((methyl-d₃)amino)-2,7- | |
| | naphthyridin-3- | |
| | yl)cyclopropanecarboxamide | |

### Example 514: Synthesis of N-(8-(ethylamino)-5-(6-morpholino-[1,2,4]triazolo[1,5-a]pyridin-2-yl)-2,7-naphthyridin-3-yl)cyclopropanecarboxamide

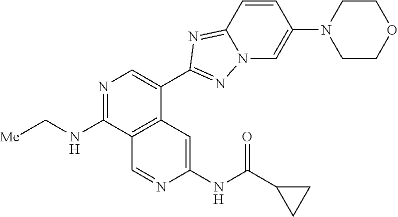 [SMILES: CCNc1ncc(-c2nc3ccc(N4CCOCC4)cn3n2)c2cc(NC(=O)C3CC3)ncc12]

### Step 1: N-(8-(ethylamino)-5-(4,4,5,5-tetramethyl-1,3,2-dioxaborolan-2-yl)-2,7-naphthyridin-3-yl)cyclopropanecarboxamide

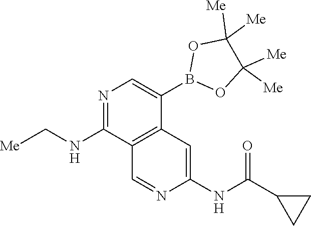 [SMILES: CCNc1ncc(B2OC(C)(C)C(C)(C)O2)c2cc(NC(=O)C3CC3)ncc12]

A mixture of N-(5-bromo-8-(ethylamino)-2,7-naphthyridin-3-yl)cyclopropanecarboxamide (500.0 mg; 1.492 mmol; 1.00 eq.), 4,4,4′,4′,5,5,5′,5′-octamethyl-2,2′-bi(1,3,2-dioxaborolane) (1.89 g; 7.458 mmol; 5.00 eq.), Pd(dppf)Cl₂·CH₂Cl₂ (243.0 mg; 0.298 mmol; 0.20 eq.) and KOAc (292.8 mg; 2.983 mmol; 2.00 eq.) in 1,4-dioxane (10 mL) under nitrogen atmosphere. The mixture was stirred at 100° C. for 2 hours. The desired product was detected via LCMS. The resulting mixture was concentrated under reduced pressure. The residue was dissolved with CH₂Cl₂ (15 mL), the insoluble solids were removed by filtration. Most of the solvent in filtrate was removed under vacuum, and the remaining 2 mL of solution was added dropwise into n-hexane (50 mL) and precipitates were formed. The solids were collected by filtration to afford N-(8-(ethylamino)-5-(4,4,5, 5-tetramethyl-1,3,2-dioxaborolan-2-yl)-2,7-naphthyridin-3-yl)cyclopropanecarboxamide as a brown solid (400.0 mg, 51.9%). LCMS (ESI) m/z 383.2 [M+H]⁺.

### Step 2: N-(8-(ethylamino)-5-(6-morpholino-[1,2,4]triazolo[1,5-a]pyridin-2-yl)-2,7-naphthyridin-3-yl)cyclopropanecarboxamide

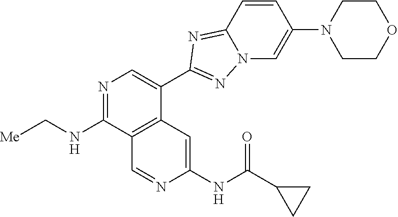 [SMILES: CCNc1ncc(-c2nc3ccc(N4CCOCC4)cn3n2)c2cc(NC(=O)C3CC3)ncc12]

A mixture of N-(8-(ethylamino)-5-(4,4,5,5-tetramethyl-1,3,2-dioxaborolan-2-yl)-2,7-naphthyridin-3-yl)cyclopropanecarboxamide(481.3 mg; 1.260 mmol; 1.00 eq.) and 4-(2-chloro-[1,2,4]triazolo[1,5-a]pyridin-6-yl)morpholine (300.0 mg; 1.257 mmol; 1.00 eq.), XPhos Pd G₃ (160.6 mg; 0.189 mmol; 0.15 eq.), XPhos (90.1 mg; 0.189 mmol; 0.15 eq.), K₃PO₄ (801.3 mg; 3.780 mmol; 3.00 eq.) in a mixture solvent of 1,4-dioxane/water (5:1, 24 mL) was stirred at 110° C. for 1 hour under nitrogen atmosphere. The desired product was detected via LCMS. The resulting mixture was concentrated under reduced pressure. The residue was purified by flash chromatography on silica gel column using 1-6% of MeOH in CH₂Cl₂ as eluent to provide N-(8-(ethylamino)-5-(6-morpholino-[1,2,4]triazolo[1,5-a]pyridin-2-yl)-2,7-naphthyridin-3-yl)cyclopropanecarboxamide as an off-white solid (180 mg, 31.2%). LCMS (ESI) m/z 459.2, [M+H]⁺. ¹H NMR (400 MHz, DMSO-d₆) δ 10.93 (s, 1H), 9.44 (s, 1H), 9.38 (s, 1H), 8.69 (s, 1H), 8.33 (d, J=2.0 Hz, 1H), 8.20-8.15 (m, 1H), 7.74 (d, J=9.6 Hz, 1H), 7.67 (dd, J=9.6, 2.0 Hz, 1H), 3.83-3.77 (m, 4H), 3.65-3.57 (m, 2H), 3.19-3.13 (m, 4H), 2.12-2.04 (m, 1H), 1.27 (t, J=7.2 Hz, 3H), 0.89-0.82 (m,

### Examples 515-517

Each compound in Table 20 below was prepared using a similar experimental procedure to prepare in Example 514, using N-(8-(ethylamino)-5-(4,4,5,5-tetramethyl-1,3,2-dioxaborolan-2-yl)-2,7-naphthyridin-3-yl)cyclopropanecarboxamide as the common intermediate and appropriate 2-chloro-[1,2,4]triazolo[1,5-a]pyridine substrates.

TABLE 20

| Example | Structure and Name | MS and ¹H NMR |
|---|---|---|
| Example 515 | 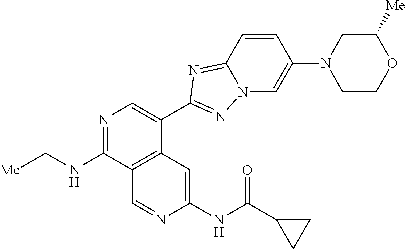 | LCMS (ESI) m/z 473.2, [M + H]⁺. ¹H NMR (400 MHz, DMSO-d₆) δ 10.95 (s, 1H), 9.46 (s, 1H), 9.39 (s, 1H), 8.68 (s, 1H), 8.33 (d, J = 2.0 Hz, 1H), 8.30- 8.17 (m, 1H), 7.74 (d, J = 9.6 Hz, 1H), 7.68 (dd, J = 9.6, 2.0 Hz, 1H), 4.00-3.90 (m, 1H), 3.75-3.57 (m, 5H), 3.54-3.47 (m, 1H), 2.80-2.69 (m, 1H), 2.47-2.37 (m, 1H), 2.12- 2.02 (m, 1H), 1.27 (d, J = 7.2 Hz, 3H), 1.17 (d, J = 6.0 Hz, 3H), 0.89-0.80 (m, 4H). |
| | (S)-N-(8-(ethylamino)-5-(6-(2- | |
| | methylmorpholino)- | |
| | [1,2,4]triazolo[1,5-a]pyridin-2- | |
| | yl)-2,7-naphthyridin-3- | |
| | yl)cyclopropanecarboxamide | |
| Example 516 | 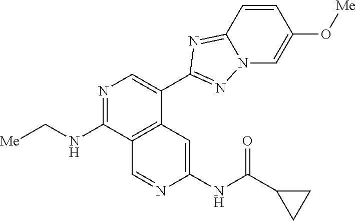 | LCMS (ESI) m/z 404.2, [M + H]⁺. ¹H NMR (400 MHz, DMSO-d₆) δ 10.94 (s, 1H), 9.45 (s, 1H), 9.40 (s, 1H), 8.71 (s, 1H), 8.67 (d, J = 2.0 Hz, 1H), 8.25- 8.16 (m, 1H), 7.78 (d, J = 9.6 Hz, 1H), 7.46 (dd, J = 9.6, 2.0 Hz, 1H), 3.91 (s, 3H), 3.67-3.56 (m, 2H), 2.12- 2.01 (m, 1H), 1.27 (d, J = 7.2 Hz, 3H), 0.89-0.80 (m, 4H). |
| | N-(8-(ethylamino)-5-(6-methoxy- | |
| | [1,2,4]triazolo[1,5-a]pyridin-2- | |
| | yl)-2,7-naphthyridin-3- | |
| | yl)cyclopropanecarboxamide | |
| Example 517 | 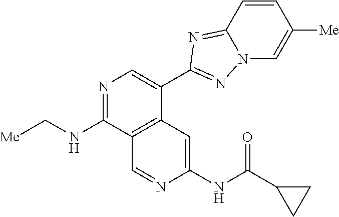 | LCMS (ESI) m/z 388.2, [M + H]⁺. ¹H NMR (400 MHz, DMSO-d₆) δ 10.95 (s, 1H), 9.48 (s, 1H), 9.45 (s, 1H), 8.79- 8.77 (m, 2H), 8.23-8.18 (m, 1H), 7.76 (d, J = 9.2 Hz, 1H), 7.55 (dd, J = 9.2, 1.6 Hz, 1H), 3.64-3.60 (m, 2H), 2.41 (s, 3H), 2.11-2.05 (m, 1H), 1.28 (t, J = 7.2 Hz, 3H), 0.88-0.81 (m, 4H). |
| | N-(8-(ethylamino)-5-(6-methyl- | |
| | [1,2,4]triazolo[1,5-a]pyridin-2- | |
| | yl)-2,7-naphthyridin-3- | |
| | yl)cyclopropanecarboxamide | |

### Example 518: Synthesis of N-(8-(cyclopropylamino)-5-(6-morpholino-[1,2,4]triazolo[1,5-a]pyridin-2-yl)-2,7-naphthyridin-3-yl)cyclopropanecarboxamide

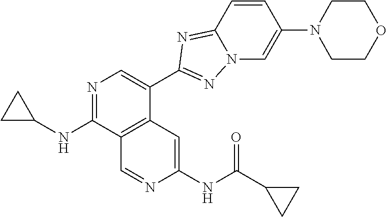

### Step 1: N-(8-(cyclopropylamino)-5-(4,4,5,5-tetramethyl-1,3,2-dioxaborolan-2-yl)-2,7-naphthyridin-3-yl)cyclopropanecarboxamide

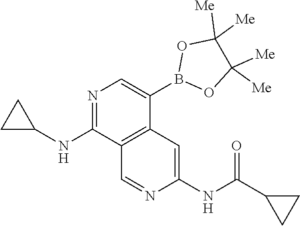 [SMILES: CC1(C)OB(c2cnc(NC3CC3)c3cnc(NC(=O)C4CC4)cc23)OC1(C)C]

A mixture of N-(5-bromo-8-(cyclopropylamino)-2,7-naphthyridin-3-yl)cyclopropanecarboxamide (200.0 mg; 0.576 mmol; 1.00 eq.), and Pin₂B₂ (1.46 g; 5.760 mmol; 10.00 eq.) in 1,4-dioxane (10 mL) under nitrogen atmosphere. To the above mixture were added Pd(dppf)Cl₂·CH₂Cl₂ (46.9 mg; 0.058 mmol; 0.10 eq.) and KOAc (113.0 mg; 1.152 mmol; 2.00 eq.). The resulting mixture was stirred at 100° C. overnight. The desired product was detected via LCMS. The mixture was allowed to cool down to room temperature and concentrated under reduced pressure. The residue was dissolved in a mixture of petroleum ether/EtOAc (10:1, 15 mL) and the precipitated product was isolated via filtration and washed with a mixture solvent of petroleum ether/EtOAc(10:1, 5 mL). The solids were dried under reduced pressure to afford N-(8-(cyclopropylamino)-5-(4,4,5,5-tetramethyl-1,3,2-dioxaborolan-2-yl)-2,7-naphthyridin-3-yl)cyclopropanecarboxamide as a brown solid (300.0 mg, crude). LCMS (ESI) m/z 395.2, [M+H]⁺.

### Step 2: N-(8-(cyclopropylamino)-5-(6-morpholino-[1,2,4]triazolo[1,5-a]pyridin-2-yl)-2,7-naphthyridin-3-yl)cyclopropanecarboxamide

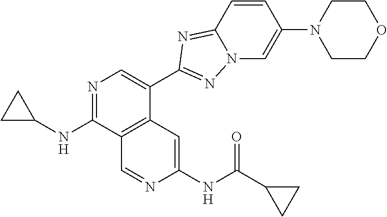 [SMILES: O=C(Nc1cc2c(-c3nc4ccc(N5CCOCC5)cn4n3)cnc(NC3CC3)c2cn1)C1CC1]

A mixture of N-(8-(cyclopropylamino)-5-(4,4,5,5-tetramethyl-1,3,2-dioxaborolan-2-yl)-2,7-naphthyridin-3-yl)cyclopropanecarboxamide (264.3 mg; 0.670 mmol; 1.00 eq.), XPhos Pd G₃ (170.2 mg; 0.201 mmol; 0.30 eq.), XPhos (95.8 mg; 0.201 mmol; 0.30 eq.), K₃PO₄ (426.8 mg; 2.010 mmol; 3.00 eq.) and 4-(2-chloro-[1,2,4]triazolo[1,5-a]pyridin-6-yl)morpholine (160.0 mg; 0.670 mmol; 1.00 eq.) in a mixture of 1,4-dioxane/water (5:1, 9.6 mL) was stirred at 90° C. for 1 hour under nitrogen atmosphere. The desired product was detected via LCMS. The resulting mixture was concentrated under reduced pressure. The residue was purified by flash chromatography on silica gel column using 5-10% of MeOH in CH₂Cl₂ as eluent to afford the crude product. The product was purified by flash chromatography on pre-packed C18 column using 20-100% of MeOH in water (10 mmol/L NH₄HCO₃) as eluent to provide N-(8-(cyclopropylamino)-5-(6-morpholino-[1,2,4]triazolo[1,5-a]pyridin-2-yl)-2,7-naphthyridin-3-yl)cyclopropanecarboxamide as a yellow solid (50.7 mg, 16.0%). LCMS (ESI) m/z 471.2, [M+H]⁺. ¹H NMR (400 MHz, DMSO-d₆) δ 10.92 (s, 1H), 9.42 (s, 1H), 9.39 (s, 1H), 8.74 (s, 1H), 8.38-8.28 (m, 1H), 8.25-8.11 (m, 1H), 7.79-7.63 (m, 2H), 3.87-3.71 (m, 4H), 3.20-3.12 (m, 41-1), 3.12-3.03 (m, 1H), 2.14-1.99 (m, 1H), 0.94-0.60 (m, 8H).

### Example 519: Synthesis of N-(5-([1,2,4]triazolo[1,5-a]pyridin-2-yl)-8-(cyclopropylamino)-2,7-naphthyridin-3-yl)cyclopropanecarboxamide

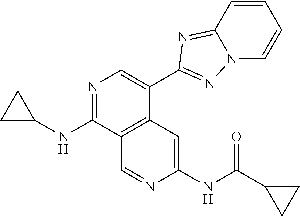 [SMILES: O=C(Nc1cc2c(-c3nc4ccccn4n3)cnc(NC3CC3)c2cn1)C1CC1]

A mixture of N-(8-(cyclopropylamino)-5-(4,4,5,5-tetramethyl-1,3,2-dioxaborolan-2-yl)-2,7-naphthyridin-3-yl)cyclopropanecarboxamide (Example 518, Step 1) (205.4 mg; 0.521 mmol, 1.00 eq.), 2-chloro-[1,2,4]triazolo[1,5-a]pyridine (80.0 mg; 0.521 mmol, 1.00 eq.), Mhos Pd G₃ (132.3 mg; 0,156 mmol; 0.30 eq.), XPhos (74.5 mg; 0.156 mmol; 0.30 eq.) and K₃PO₄ (331.7-1.563 mmol; 3.00 eq.) in a mixture of 1,4-dioxane/water (5:1, 9 mL) was stirred at 110° C. for 1 hour under nitrogen atmosphere. The desired product was detected via LCMS. The resulting mixture was concentrated under reduced pressure. The residue was purified by flash chromatography on silica gel column using 5-10% of MeOH in CH₂Cl₂ as eluent to afford the crude product. The crude product was purified by Prep-Achiral-SFC (Column: Viridis BEH Prep 2-EP OBD Column 30×150 mm, 5 μm; Mobile Phase A: CO₂, Mobile Phase B: MeOH (0.1% 2 M NH₃-MeOH); Flow rate: 60 ma/min; Gradient: isocratic 25% B) to afford N-(5-([1,2,4]triazolo[1,5-a]pyridin-2-34)-8-(cyclopropylamino)-2,7-naphthyridin-3-yl)cyclopropanecarboxamide as a yellow solid (1.6.2 mg, 8.0%). LCMS (ESI) m/z 386.2, [M+H]⁺.

### Example 520: Synthesis of (1S,2R)—N-(5-(6-methoxy-[1,2,4]triazolo[1,5-a]pyridin-2-yl)-8-(methylamino)-2,7-naphthyridin-3-yl)-2-methylcyclopropane-1-carboxamide

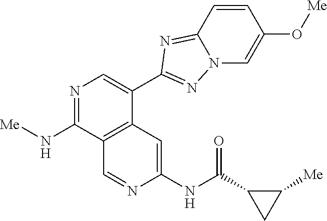 [SMILES: CNc1ncc(-c2nc3ccc(OC)cn3n2)c2cc(NC(=O)[C@H]3C[C@H]3C)ncc12]

### Step 1: 4-(6-methoxy-[1,2,4]triazolo[1,5-a]pyridin-2-yl)-N¹-methyl-2,7-naphthyridine-1,6-diamine

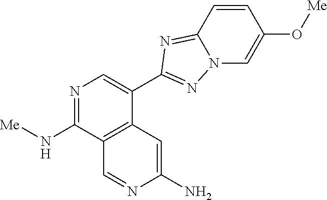 [SMILES: CNc1ncc(-c2nc3ccc(OC)cn3n2)c2cc(N)ncc12]

To a solution of N-(5-(6-methoxy-[1,2,4]triazolo[1,5-a]pyridin-2-yl)-8-(methylamino)-2,7-naphthyridin-3-yl)cyclopropanecarboxamide (Example 470) (280.0 mg; 0.719 mmol; 1.00 eq.) in a mixture solvent of DMSO/MeOH (1:5, 18 mL) was added a solution of NaOH (287.6 mg; 7.191 mmol; 10.00 eq.) in water (4.5 mL) dropwise at 0° C. The resulting mixture was stirred at 60° C. overnight. The desired product was detected via LCMS. The resulting mixture was concentrated under reduced pressure. The residue was purified by flash chromatography on pre-packed C18 column using 40-50% of MeOH in water (10 mmol/L NH₄HCO₃) as eluent to provide 4-(6-methoxy-[1,2,4]triazolo[1,5-a]pyridin-2-yl)-N¹-methyl-2,7-naphthyridine-1,6-diamine as a yellow solid (210.0 mg, 90.8%). LCMS (ESI) m/z 322.1, [M+H]⁺.

### Step 2: (1S,2R)—N-(5-(6-methoxy-[1,2,4]triazolo[1,5-a]pyridiin-2-yl)-8-(methylamino)-2,7-naphthyridin-3-yl)-2-methylcyclopropane-1-carboxamide

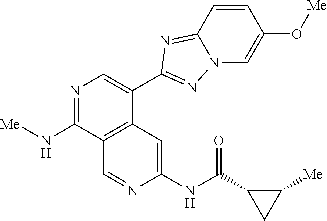 [SMILES: CNc1ncc(-c2nc3ccc(OC)cn3n2)c2cc(NC(=O)[C@H]3C[C@H]3C)ncc12]

To a stirred solution of 4-(6-methoxy-[1,2,4]triazolo[1,5-a]pyridin-2-yl)-N¹-methyl-2,7-naphthyridine-1,6-diamine (70.0 mg; 0.218 mmol; 1.00 eq.) and (1S,2R)-2-methylcyclopropane-1-carboxylic acid (19.6 mg; 0.196 mmol; 0.90 eq.) in pyridine (3 mL) was added POCl₃ (100.2 mg; 0.654 mmol; 3.00 eq.) dropwise at 0° C. The resulting mixture was stirred at room temperature for 0.5 hour under nitrogen atmosphere. The desired product was detected via LCMS. The reaction was quenched with water at 0′C. The resulting mixture was concentrated under reduced pressure. The residue was purified by flash chromatography on silica gel column using 1-6% of MeOH in CH₂Cl₂ as eluent to provide the crude product. The product was purified by flash chromatography on pre-packed C18 column using 70-90% of MeOH in water (10 mmol/L NH₄HCO₃) as eluent to provide (1S,2R)—N-(5-(6-methoxy-[1,2,4]triazolo[1,5-a]pyridin-2-yl)-8-(methylamino)-2,7-naphthyridin-3-yl)-2-methylcyclopropane-1-carboxamide as a white solid (32.5 mg, 36.9%). LCMS (ESI) m/z 404.2, [M+H]⁺. ¹H NMR (400 MHz, DMSO-d₆) δ 10.81 (s, 1H), 9.40 (s, 1H), 9.39 (s, 1H), 8.73 (s, 1H), 8.69 (d, J=2.0 Hz, 1H), 8.30-8.23 (m, 1H), 7.79 (d, J=9.6 Hz, 1H), 7.47 (dd, J=9.6, 2.0 Hz, 1H), 3.91 (s, 3H), 3.06 (d, J=4.4 Hz, 3H), 2.13-2.03 (m, 1H), 1.34-1.22 (m, 1H), 1.13 (d, J=6.0 Hz, 3H), 1.03-0.96 (m, 1H), 0.87-0.81 (m, 1H).

### Examples 521 and 522: Synthesis of (1S,2S)-2-ethyl-N-(5-(6-methoxy-[1,2,4]triazolo[1,5-a]pyridin-2-yl)-8-(methylamino)-2,7-naphthyridin-3-yl)cyclopropane-1-carboxamide (Example 521) and (1R,2R)-2-ethyl-N-(5-(6 methoxy-[1,2,4]triazolo[1,5-a]pyridin-2-yl)-8-(methylamino)-2,7-naphthyridin-3-yl)cyclopropane-1-carboxamide (Example 522)

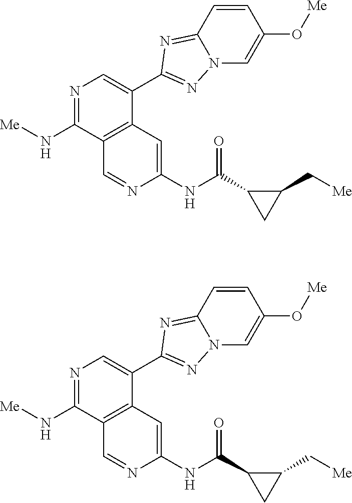 [SMILES: CC[C@H]1C[C@@H]1C(=O)Nc1cc2c(-c3nc4ccc(OC)cn4n3)cnc(NC)c2cn1 ; CC[C@@H]1C[C@H]1C(=O)Nc1cc2c(-c3nc4ccc(OC)cn4n3)cnc(NC)c2cn1]

To a stirred solution of 4-(6-methoxy-[1,2,4]triazolo[1,5-a]pyridin-2-yl)-N¹-methyl-2,7-naphthyridine-1,6-diamine (Example 520, Step 1) (98.0 mg; 0.31 mmol; 1.00 eq.) and 2-ethylcyclopropane-1-carboxylic acid (trans racemate) (31.3 mg; 0.274 mmol; 0.90 eq.) in pyridine (4 mL) was added POCl₃ (140.1 mg; 0.91 mmol; 3.00 eq.) at 0° C. The reaction was stirred at room temperature for 0.5 hour under nitrogen atmosphere. The desired product was detected via LCMS. The resulting mixture was diluted with EtOAc (90 mL), washed with brine (3×4 mL). The organic layer was dried over anhydrous Na₂SO₄, filtered, and concentrated under reduced pressure. The residue was purified by flash chromatography on silica gel column using 2-10% of MeOH in CH₂Cl₂ as eluent to provide the trans racemate. The trans racemate was separated by Prep-Chiral-HPLC (Column: CHIRAL ART Cellulose-SC, 2×25 cm, 5 μm; Mobile Phase A: Hex (0.5% 2 M NH₃-MeOH)-HPLC, Mobile Phase B: EtOH: CH₂Cl₂=1:1; Flow rate: 20 mL/min; Gradient: 50% B to 50% B in 1.2.5 min) to afford (1S,2S)-2-ethyl-N-(5-(6-methoxy-[1,2,4]triazolo[1,5-a]pyridin-2-yl)-8-(methylamino)-2,7-naphthyridin-3-yl)cyclopropane-1-carboxamide (Example 521, the faster peak) as a yellow solid (23.9 mg, 18.7%) and (1R,2R)-2-ethyl-N-(5-(6-methoxy-[1,2,4]triazolo[1,5-a]pyridin-2-yl)-8-(methylamino)-2,7-naphthyridin-3-yl)cyclopropane-1-carboxamide (Example 522, the slower peak) as a yellow solid (27.4 mg, 21.5%). The two configurations are arbitrarily assigned. LCMS (ESI) m/z 418.2, [M+H]⁺.

HNMR for Example 521: ¹H NMR (400 MHz, DMSO-d₆) δ 10.88 (s, 1H), 9.41 (s, 1H), 9.40 (s, 1H), 8.72 (s, 1H), 8.67 (d, J=2.0 Hz, 1H), 8.33-8.20 (m, 1H), 7.78 (d, J=9.6 Hz, 1H), 7.46 (dd, J=9.6, 2.0 Hz, 1H), 3.91 (s, 3H), 3.05 (d, J=4.4 Hz, 3H), 1.91-1.83 (m, 1H), 1.46-1.21 (m, 3H), 1.09-0.93 (m, 4H), 0.76-0.67 (m, 1H).

HNMR for Example 522: ¹H NMR (400 MHz, DMSO-d₆) δ 10.88 (s, 1H), 9.41 (s, 1H), 9.40 (s, 1H), 8.72 (s, 1H), 8.67 (d, J=2.0 Hz, 1H), 8.33-8.20 (m, 1H), 7.78 (d, J=9.6 Hz, 1H), 7.46 (dd, J=9.6, 2.0 Hz, 1H), 3.91 (s, 3H), 3.05 (d, J=4.4 Hz, 3H), 1.91-1.83 (m, 1H), 1.46-1.21 (m, 3H), 1.09-0.93 (m, 4H), 0.76-0.67 (m, 1H).

### Example 523: Synthesis of 4-(6-methoxy-[1,2,4]triazolo[1,5-a]pyridin-2-yl)-N¹-methyl-N⁶-(pyridin-2-yl)-2,7-naphthyridine-1,6-diamine

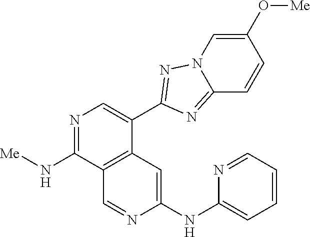 [SMILES: CNc1ncc(-c2nc3ccc(OC)cn3n2)c2cc(Nc3ccccn3)ncc12]

To a stirring mixture of Cs₂CO₃ (200.0 mg; 0.614 mmol; 2.19 eq.), Xantphos (40.0 mg; 0.069 mmol; 0.25 eq.) and Pd₂(dba)₃ (50.0 mg; 0.055 mmol; 0.19 eq.) in 1,4-dioxane (8 mL) was added 4-(6-methoxy-[1,2,4]triazolo[1,5-a]pyridin-2-yl)-N¹-methyl-2,7-naphthyridine-1,6-diamine (Example 520, Step 1) (90.0 mg; 0.280 mmol; 1.00 eq.) and 2-chloropyridine (35.0 mg; 0.308 mmol; 1.10 eq.) under nitrogen atmosphere. The resulting solution was stirred at 130° C. for 12 hours. The desired product was detected via LCMS. The resulting solution was concentrated under vacuum. The residue was purified by by flash chromatography, on silica gel column, using 6-10% of MeOH in CH₂Cl₂ to afford the crude product. The crude product was purified by flash chromatography on pre-packed C18 column using 40-70% of MeCN in water (10 mmol/L NH₄HCO₃) as eluent to provide 4-(6-methoxy-[1,2,4]triazolo[1,5-a]pyridin-2-yl)-N¹-methyl-N⁶-(pyridin-2-yl)-2,7-naphthyridine-1,6-diamine as a light yellow solid (36.2 mg, 32.4%). LCMS (ESI) m/z 399.2, [M+H]⁺. ¹H NMR (300 MHz, DMSO-d₆) δ 9.89 (s, 1H), 9.56 (s, 1H), 9.34 (s, 1H), 8.75 (s, 1H), 8.67 (d, J=2.8 Hz, 1H), 8.37-8.28 (m, 1H), 8.27-8.19 (m, 1H), 7.82-7.75 (m, 1H), 7.73-7.68 (m, 1H), 7.51-7.42 (m, 2H), 6.94-6.88 (m, 1H), 3.92 (s, 3H), 3.05 (d, J=4.4 Hz, 3H).

### Example 524: Synthesis of 4-(6-methoxy-[1,2,4]triazolo[1,5-a]pyridin-2-yl)-N⁶-(6-methoxypyridin-2-yl)-N¹-methyl-2,7-naphthyridine-1,6-diamine

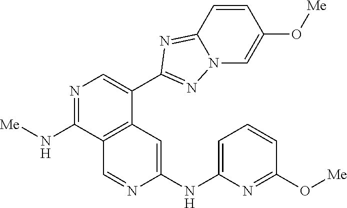 [SMILES: CNc1ncc(-c2nc3ccc(OC)cn3n2)c2cc(Nc3cccc(OC)n3)ncc12]

To a stirring mixture of 4-(6-methoxy-[1,2,4]triazolo[1,5-a]pyridin-2-yl)-N¹-methyl-2,7-naphthyridine-1,6-diamine (Example 520, Step 1) (90.0 mg; 0.280 mmol; 1.00 eq.) in 1,4-dioxane (3 mL) were added 2-chloro-6-methoxypyridine (40.6 mg; 0.283 mmol; 1.01 eq.), Pd₂(dba)₃ (25.6 mg; 0.028 mmol; 0.10 eq.), XantPhos (32.4 mg; 0.056 mmol; 0.20 eq.) and Cs₂CO₃ (182.5 mg; 0.560 mmol; 2.00 eq.). The reaction was stirred at 130° C. for 3 hours under nitrogen atmosphere. The desired product was detected via LCMS. The mixture was concentrated under pressure. The residue was purified by flash chromatography on silica gel column using 1-6% of MeOH in CH₂Cl₂ as eluent to provide the crude product. The product was repurified by flash chromatography on pre-packed C18 column using 10-80% of MeOH in water (10 mmol/L NH₄HCO₃) as eluent to provide 4-(6-methoxy-[1,2,4]triazolo[1,5-a]pyridin-2-yl)-N⁶-(6-methoxypyridin-2-yl)-N¹-methyl-2,7-naphthyridine-1,6-diamine as an off-white solid (30.0 mg, 24.9%). LCMS (ESI) m/z 429.2, [M+H]⁺. ¹H NMR (400 MHz, DMSO-d₆) δ 9.87 (s, 1H), 9.35 (s, 1H), 9.17 (s, 1H), 8.70 (d, J=2.0 Hz, 1H), 8.61 (s, 1H), 8.18-8.10 (m, 1H), 7.76 (d, J=9.6 Hz, 1H), 7.62-7.53 (m, 1H), 7.48 (dd, J=9.6, 2.0 Hz, 1H), 6.87 (d, J=8.0 Hz, 1H), 6.28 (d, J=8.0 Hz, 1H), 3.89 (s, 3H), 3.52 (s, 3H), 3.05 (d, J=4.4 Hz, 3H).

### Example 525: Synthesis of (1S,2R)-2-methyl-N-(8-(methylamino)-5-(6-morpholino-[1,2,4]triazolo[1,5-a]pyridin-2-yl)-2,7-naphthyridin-3-yl)cyclopropane-1-carboxamide

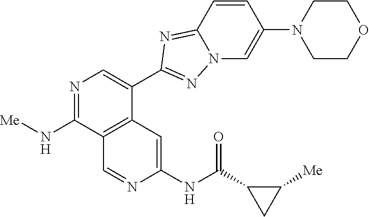 [SMILES: CNc1ncc(-c2nc3ccc(N4CCOCC4)cn3n2)c2cc(NC(=O)[C@H]3C[C@H]3C)ncc12]

### Step 1: N-methyl-4-(6-morpholino-[1,2,4]triazolo[1,5-a]pyridin-2-yl)-2,7-naphthyridine-1,6-diamine

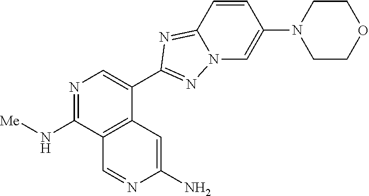 [SMILES: CNc1ncc(-c2nc3ccc(N4CCOCC4)cn3n2)c2cc(N)ncc12]

To a stirred solution of N-(8-(methylamino)-5-(6-morpholino-[1,2,4]triazolo[1,5-a]pyridin-2-yl)-2,7-naphthyridin-3-yl)cyclopropanecarboxamide (Example 471) (1.40 g; 3.151 mmol; 1.00 eq.) in a mixture of solvents: DMSO/MeOH/water (7:14:1, 37 mL) was added NaOH (1.30 g; 32.501 mmol; 10.32 eq.) at room temperature under nitrogen atmosphere. The resulting mixture was stirred at 60° C. for 16 hours. The desired product was detected via LCMS. The resulting mixture was concentrated under reduced pressure to remove MeOH. To the remaining mixture was added water (50 mL) and precipitates were formed. The precipitated solids were collected by filtration and washed with water. The solids were dried under reduced pressure to afford M-methyl-4-(6-morpholino-[1,2,4]triazolo[1,5-a]pyridin-2-yl)-2,7-naphthyridine-1,6-diamine as a brown solid (1.02 g, 84.3%). LCMS (ESI) m/z 377.2, [M+H]⁺.

Each intermediate in Table 21 below was prepared using a similar experimental procedure to prepare N¹-methyl-4-(6-morpholino-[1,2,4]triazolo[1, 5-a]pyridin-2-yl)-2,7-naphthyridine-1,6-diamine, where N-(8-(methylamino)-5-(6-morpholino-[1,2,4]triazolo[1,5-a]pyridin-2-yl)-2,7-naphthyridin-3-yl)cyclopropanecarboxamide was replaced with the starting material as shown Table 21 below.

| Intermediate | Starting material | MS |
|---|---|---|
| 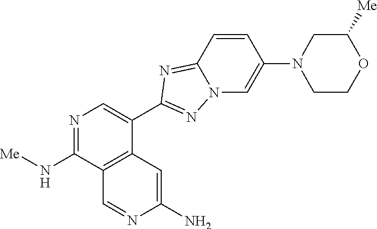 | (S)-N-(8-(methylamino)-5-(6- (2-methylmorpholino)- [1,2,4]triazolo[1,5-a]pyridin- 2-yl)-2,7-naphthyridin-3- yl)cyclopropanecarboxamide | LCMS (ESI) m/z 391.0, [M + H]⁺. |
| 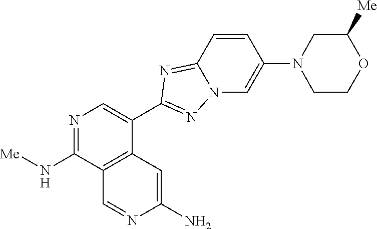 | (R)-N-(8-(methylamino)-5-(6- (2-methylmorpholino)- [1,2,4]triazolo[1,5-a]pyridin- 2-yl)-2,7-naphthyridin-3- yl)cyclopropanecarboxamide | LCMS (ESI) m/z 391.2. [M + H]⁺. |

### Step 2: (1S,2R)-2-methyl-N-(8-(methylamino)-5-(6-morpholino-[1,2,4]triazolo[1,5-a]pyridin-2-yl)-2,7-naphthyridin-3-yl)cyclopropane-1-carboxamide

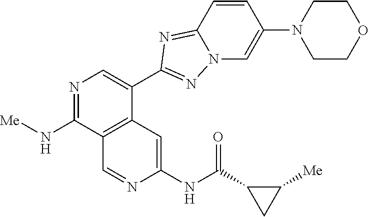 [SMILES: CNc1ncc(-c2nc3ccc(N4CCOCC4)cn3n2)c2cc(NC(=O)[C@H]3C[C@H]3C)ncc12]

To a stirred solution of N1-methyl-4-(6-morpholino-[1,2,4]triazolo[1,5-a]pyridin-2-yl)-2,7-naphthyridine-1,6-diamine (34 mg; 0.09 mmol; 1.00 eq.) and (1S,2R)-2-methylcyclopropane-1-5 carboxylic acid (8.1 mg; 0.08 mmol; 0.90 eq.) in pyridine (0.7 mL) was added POCl₃ (41 mg; 0.27 mmol; 3.00 eq.) dropwise at 0° C. under nitrogen atmosphere. The mixture was stirred at room temperature for 0.5 hour. The mixture was slowly quenched with water (1 mL) and basified to pH=7 with saturated NaHCO₃ solution. The resulting mixture was extracted with EtOAc (3×10 mL), the combined organic phase was concentrated under vacuum. The residue was purified by flash chromatography on pre-packed C18 column using 20-100% of MeCN/THF=3:1 in water (10 mmol/L NH₄HCO₃) as eluent to provide (1S,2R)-2-methyl N-(8-(methyl amino)-5-(6-morpholino-[1,2,4]triazolo[1,5-a]pyridin-2-yl)-2,7-naphthyridin-3-yl)cyclopropane-1-carboxamide (23 mg, 56.0%) as an off-white solid.

### Examples 526-531

Each compound in Table 22 below was prepared using a similar experimental procedure to prepare Example 525, using N¹-methyl-4-(6-morpholino-[1,2,4]triazolo[1,5-a]pyridin-2-yl)-2,7-naphthyridine-1,6-diamine as the common intermediate and appropriate acid.

TABLE 22

| Example | Structure and Name | MS and ¹H NMR |
|---|---|---|
| Example 526 | 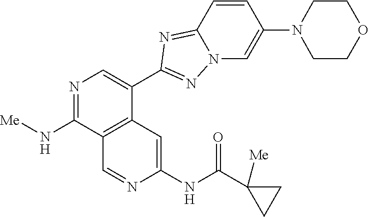 | LCMS (ESI) m/z 459.2, [M + H]⁺. ¹H NMR (400 MHz, DMSO-d₆) δ 9.60 (s, 1H), 9.42 (s, 1H), 9.36 (s, 1H), 8.74 (s, 1H), 8.35 (d, J = 2.0 Hz, 1H), 8.30- 8.25 (m, 1H), 7.75 (d, J = 9.6 Hz, 1H), 7.68 (dd, J = 9.6, 2.0 Hz, 1H), 3.80- 3.78 (m, 4H), 3.18-3.15 (m, 4H), 3.05 (d, J = 4.4 Hz, 3H), 1.46 (s, 3H), 1.18-1.15 (m, 2H), 0.70-0.68 (m, 2H). |
| | 1-methyl-N-(8-(methylamino)-5- | |
| | (6-morpholino- | |
| | [1,2,4]triazolo[1,5-a]pyridin-2- | |
| | yl)-2,7-naphthyridin-3- | |
| | yl)cycloproapne-1-carboxamide | |
| Example 527 | 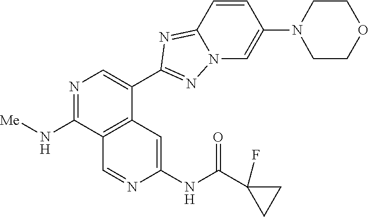 | LCMS (ESI) m/z 463.2, [M + H]⁺. ¹H NMR (400 MHz, DMSO-d₆) δ 10.32 (s, 1H), 9.46 (s, 1H), 9.44 (s, 1H), 8.80 (s, 1H), 8.35 (d, J = 2.0 Hz, 1H), 8.34- 8.31 (m, 1H), 7.75 (d, J = 9.6 Hz, 1H), 7.69 (dd, J = 9.6, 2.0 Hz, 1H), 3.87-3.66 (m, 4H), 3.20-3.13 (m, 4H), 3.07 (d, J = 4.4 Hz, 3H), 1.52- 1.36 (m, 4H). |
| | 1-fluoro-N-(8-(methylamino)-5- | |
| | (6-morpholino- | |
| | [1,2,4]triazolo[1,5-a]pyridin-2- | |
| | yl)-2,7-naphthyridin-3- | |
| | yl)cyclopropane-1-carboxamide | |
| Example 528 | 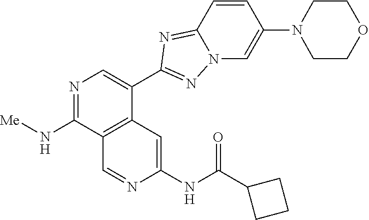 | LCMS (ESI) m/z 459.2, [M + H]⁺. ¹H NMR (400 MHz, DMSO-d₆) δ 10.43 (s, 1H), 9.46 (s, 1H), 9.38 (s, 1H), 8.73 (s, 1H), 8.36 (d, J = 2.0 Hz, 1H), 8.26- 8.21 (m, 1H), 7.77 (d, J = 9.6 Hz, 1H), 7.70 (dd, J = 9.6, 2.0 Hz, 1H), 3.83-3.77 (m, 4H), 3.49-3.36 (m, 1H), 3.21-3.15 (m, 4H), 3.05 (d, J = 4.4 Hz, 3H), 2.33-2.06 (m, 4H), 2.03- 1.78 (m, 2H). |
| | N-(8-(methylamino)-5-(6- | |
| | morpholino-[1,2,4]triazolo[1,5- | |
| | a]pyridin-2-yl)-2,7-naphthyridin- | |
| | 3-yl)cyclobutanecarboxamide | |
| Example 529 | 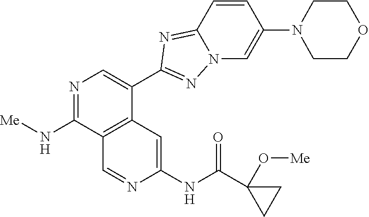 | LCMS (ESI) m/z 475.2, [M + H]⁺. ¹H NMR (400 MHz, DMSO-d₆) δ 9.62 (s, 1H), 9.46 (s, 1H), 9.43 (s, 1H), 8.79 (s, 1H), 8.39-8.27 (m, 2H), 7.82-7.60 (m, 2H), 3.87-3.73 (m, 4H), 3.41 (s, 3H), 3.22-3.12 (m, 4H), 3.06 (d, J = 4.4 Hz, 3H), 1.33-1.16 (m, 4H). |
| | 1-methoxy-N-(8-(methylamino)- | |
| | 5-(6-morpholino- | |
| | [1,2,4]triazolo[1,5-a]pyridin-2- | |
| | yl)-2,7-naphthyridin-3- | |
| | yl)cyclopropane-1-carboxamide | |
| Example 530 | 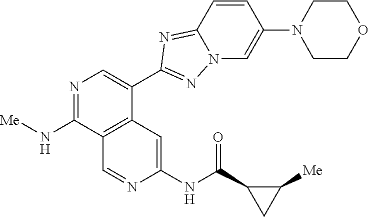 | LCMS (ESI) m/z 459.2, [M + H]⁺. ¹H NMR (400 MHz, DMSO-d₆) δ 10.85 (s, 1H), 9.41 (s, 1H), 9.39 (s, 1H), 8.67 (s, 1H), 8.34 (d, J = 2.0 Hz, 1H), 8.32- 8.20 (m, 1H), 7.76 (d, J = 9.6 Hz, 1H), 7.69 (dd, J = 9.6, 2.0 Hz, 1H), 3.82-3.78 (m, 4H), 3.20-3.15 (m, 4H), 3.06 (d, J = 4.4 Hz, 3H), 2.12- 2.03 (m, 1H), 1.36-1.22 (m, 1H), 1.12 (d, J = 6.0 Hz, 3H), 1.04-0.96 (m, 1H), 0.88-0.80 (m, 1H). |
| | (1R,2S)-2-methyl-N-(8- | |
| | (methylamino)-5-(6-morpholino- | |
| | [1,2,4]triazolo[1,5-a]pyridin-2- | |
| | yl)-2,7-naphthyridin-3- | |
| | yl)cycloproapne-1-carboxamide | |
| Example 531 | 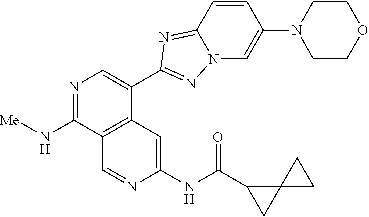 | LCMS (ESI) m/z 471.2, [M + H]⁺. |
| | N-(8-(methylamino)-5-(6- | |
| | morpholino-[1,2,4]triazolo[1,5- | |
| | a]pyridin-2-yl)-2,7-naphthyridin- | |
| | 3-yl)spiro[2.2]pentane-1- | |
| | carboxamide | |

### Examples 532 and 533: Synthesis of (R)-2-fluoro-N-(8-(methylamino)-5-(6-morpholino-[1,2,4]triazolo[1,5-a]pyridin-2-yl)-2,7-naphthyridin-3-yl)propanamide (Example 532) and (S)-2-fluoro-N-(8-(methylamino)-5-(6-morpholino-[1,2,4]triazolo[1,5-a]pyridin-2-yl)-2,7-naphthyridin-3-yl)propanamide (Example 533)

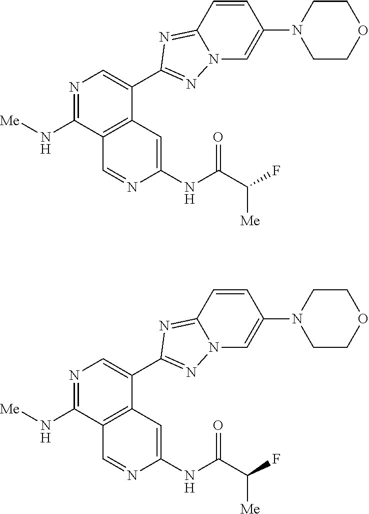 [SMILES: CNc1ncc(-c2nc3ccc(N4CCOCC4)cn3n2)c2cc(NC(=O)[C@@H](C)F)ncc12 ; CNc1ncc(-c2nc3ccc(N4CCOCC4)cn3n2)c2cc(NC(=O)[C@H](C)F)ncc12]

To a solution of N¹-methyl-4-(6-morpholino-[1,2,4]triazolo[1,5-a]pyridin-2-yl)-2,7-naphthyridine-1,6-diamine (100.0 mg; 0.27 mmol; 1.00 eq.) and 2-fluoropropanoic acid (22.0 mg; 0.24 mmol; 0.90 eq.) in pyridine mL) was added POCl₃ (122.0 mg; 0.8 mmol; 3.00 eq.) dropwise at 0° C. The reaction was stirred at room temperature for 0.5 hour under nitrogen atmosphere. The desired product was detected via LCMS. The resulting mixture was diluted with EtOAc (90 mL), washed with brine (3×5 mL). The organic layer was dried over anhydrous Na₂SO₄, filtered, and concentrated under reduced pressure. The residue was purified by flash chromatography on silica gel column using 2-10% of MeOH in CH₂C17 as eluent to provide the racemate. The racemate was separated by Prep-Chiral-HPLC (Column: CHIRALPAK IE, 2×25 cm, 5 μm; Mobile Phase A: Hex (0.5% 2 M NH₃-MeOH), Mobile Phase B: MeOH:CH₂Cl₂=1:1; Flow rate: 20 mL/min; Gradient: 40% B to 40% B in 35 min) to afford (R)-2-fluoro-N-(8-(methylamino)-5-(6-morpholino-[1,2,4]triazolo[1,5-a]pyridin-2-yl)-2,7-naphthyridin-3-yl)propanamide (Example 532, the faster peak) as a yellow solid (21.7 mg, 18.1%) and (S)-2-fluoro-N-(8-(methylamino)-5-(6-morpholino-[1,2,4]triazolo[1,5-a]pyridin-2-yl)-2,7-naphthyridin-3-yl)propanamide (Example 533, the slower peak) as a yellow solid (15.7 mg, 13.1%). The two configurations are arbitrarily assigned. LCMS (ESI) m/z 451.2, [M+H]⁺.

HNMR for Example 532: ¹H NMR (400 MHz, DMSO-d₆) δ 10.66 (s, 1.14), 9.55 (s, 1.14), 9.43 (s, 1H), 8.80 (s, 1H), 8.34 (d, J=2.0 Hz, 1H), 8.33-8.29 (m, 1H), 7.76 (d, J=9.6 Hz, 1H), 7.69 (dd, J=9.6, 2.0 Hz, 1H), 5.41-5.21 (m, 1H), 3.83-3.76 (m, 4H), 3.21-3.14 (m, 4H), 3.06 (d, J=4.4 Hz, 3H), 1.61-1.50 (m, 3H).

HNMR for Example 533: ¹H NMR (400 MHz, DMSO-d₆) 10.66 (s, 1H), 9.55 (s, 1H), 9.43 (s, 1H), 8.80 (s, 1H), 8.34 (d, J=2.0 Hz, 1H), 8.33-8.29 (m, 1H), 7.76 (d, J=9.6 Hz, 1H), 7.69 (dd, J=9.6, 2.0 Hz, 1H), 5.41-5.21 (m, 1H), 3.83-3.76 (m, 4H), 3.21-3.14 (m, 4H), 3.06 (d, J=4.4 Hz, 3H), 1.61-1.50 (m, 3H).

### Example 534 and 535: Synthesis of (1S,2R)-2-(difluoromethyl)-N-(8-(methylamino)-5-(6-morpholino-[1,2,4]triazolo[1,5-a]pyridin-2-yl)-2,7-naphthyridin-3-yl)cyclopropane-1-carboxamide (Example 534) and (1R,2S)-2-(difluoromethyl)-N-(8-(methylamino)-5-(6-morpholino-[1,2,4]triazolo[1,5-a]pyridin-2-yl)-2,7-naphthyridin-3-yl)cyclopropane-1-carboxamide (Example 535)

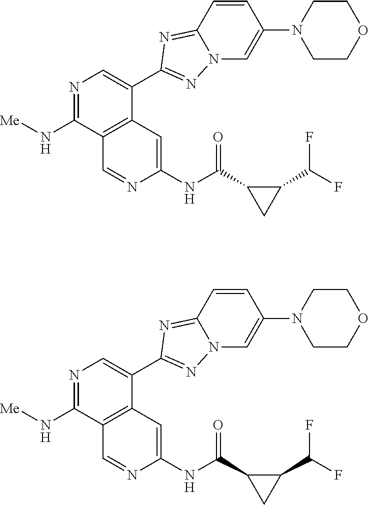 [SMILES: CNc1ncc(-c2nc3ccc(N4CCOCC4)cn3n2)c2cc(NC(=O)[C@H]3C[C@H]3C(F)F)ncc12 ; CNc1ncc(-c2nc3ccc(N4CCOCC4)cn3n2)c2cc(NC(=O)[C@@H]3C[C@@H]3C(F)F)ncc12]

To a stirred solution of N¹-methyl-4-(6-morpholino-[1,2,4]triazolo[1,5-a]pyridin-2-yl)-2,7-naphthyridine-1,6-diamine (100.0 mg; 0.27 mmol; 1.00 eq.) and 2-(difluoromethyl)cyclopropane-1-carboxylic acid (32.5 mg; 0.239 mmol; 0.90 eq.) in pyridine (3 mL) was added POCl₃ (122.0 mg; 0.80 mmol; 3.00 eq.) dropwise at 0° C. The reaction was stirred at room temperature for 0.5 hour under nitrogen atmosphere. LC-MS indicated that the reaction was complete. The mixture was diluted with EtOAc (90 mL) and washed with brine (3×5 mL). The organic layer was dried with Na₂SO₄, filtered, and the solvent was removed under reduced pressure. The residue was purified by flash chromatography, on silica gel column, using 2-10% of MeOH in CH₂Cl₂ as eluent to provide the racemic mixture. The racemic mixture was separated by Prep-Chiral-HPLC (Column: CHIRAL ART Amylose-SA, 2×25 cm, 5 μm; Mobile Phase A: Hex (0.5% 2 M NH₃-MeOH), Mobile Phase B: EtOH: CH₂Cl₂=1:1; Flow rate: 20 mL/min; Gradient: 45% B to 45% B in 17.5 min) to afford (1S,2R)-2-(difluoromethyl)-N-(8-(methylamino)-5-(6-morpholino-[1,2,4]triazolo[1,5-a]pyridin-2-yl)-2,7-naphthyridin-3-yl)cyclopropane-1-carboxamide (Example 534, the faster peak) as a yellow solid (16.9 mg, 12.8%) and (1R,2S)-2-(difluoromethyl)-N-(8-(methylamino)-5-(6-morpholino-[1,2,4]triazolo[1,5-a]pyridin-2-yl)-2,7-naphthyridin-3-yl)cyclopropane-1-carboxamide (Example 535, the slower peak) as a yellow solid (15.7 mg, 11.9%). The two configurations are arbitrarily assigned. LCMS (ESI) m/z 495.2, [M+H]⁺.

HNMR for Example 534: ¹H NMR (400 MHz, DMSO-d₆) δ 11.17 (s, 1H), 9.42 (s, 1H), 9.41 (s, 1H), 8.74 (s, 1H), 8.35 (d, J=2.0 Hz, 1H), 8.33-8.25 (m, 1H), 7.75 (d, J=9.6 Hz, 1H), 7.68 (dd, J=9.6, 2.0 Hz, 1H), 6.21-5.88 (m, 1H), 3.83-3.76 (m, 4H), 3.20-3.13 (m, 4H), 3.06 (d, J=4.4 Hz, 3H), 2.47-2.37 (m, 1H), 1.94-1.84 (m, 1H), 1.36-1.26 (m, 2H).

HNMR for Example 535: ¹H NMR (400 MHz, DMSO-d₆) δ 11.17 (s, 1H), 9.42 (s, 1H), 9.41 (s, 1H), 8.74 (s, 1H), 8.35 (d, J=2.0 Hz, 1H), 8.33-8.25 (m, 1H), 7.75 (d, J=9.6:Hz, 1H), 7.68 (dd, J=9.6, 2.0 Hz, 1H), 6.21-5.88 (m, 1H), 3.83-3.76 (m, 4H), 3.20-3.13 (m, 4H), 3.06 (d, J=4.4 Hz, 3H), 2.47-2.37 (m, 1H), 1.94-1.84 (m, 1H), 1.36-1.26 (m, 2H).

### Example 536: Synthesis of N¹-methyl-4-(6-morpholino-[1,2,4]triazolo[1,5-a]pyridin-2-yl)-N⁶-(pyridin-2-yl)-2,7-naphthyridine-1,6-diamine

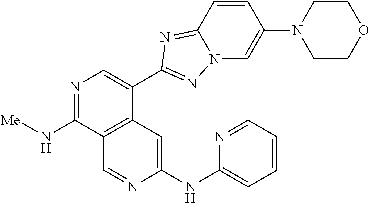 [SMILES: CNc1ncc(-c2nc3ccc(N4CCOCC4)cn3n2)c2cc(Nc3ccccn3)ncc12]

To a mixture of N¹-methyl-4-(6-morpholino-[1,2,4]triazolo[1,5-a]pyridin-2-yl)-2,7-naphthyridine-1,6-diamine (70.0 mg; 0.186 mmol; 1.00 eq.) in 1,4-dioxane (7 mL) was added Pd₂(dba)₃ (10.4 mg; 0.045 mmol; 0.24 eq.), XantPhos (51.6 mg; 0.089 mmol; 0.48 eq.), Cs₂CO₃ (121.1 mg; 0.372 mmol; 2.00 eq.) and 2-chloropyridine (21.0 mg; 0.186 mmol; 1.00 eq.). The mixture was stirred at 130° C. for 2 hours under nitrogen atmosphere. The desired product was detected via LCMS. The resulting mixture was concentrated under reduced pressure. The residue was purified by flash chromatography on silica gel column using 2-10% of MeOH in CH₂Cl₂ as eluent to provide the crude product. The crude product was purified by flash chromatography on pre-packed C18 column using 20-80% of MeOH in water (10 mmol/L NH₄HCO₃) as eluent to provide N¹-methyl-4-(6-morpholino-[1,2,4]triazolo[1,5-a]pyridin-2-yl)-N⁶-(pyridin-2-yl)-2,7-naphthyridine-1,6-diamine as a yellow solid (22.7 mg, 26.9%). LCMS (ESI) m/z 454.2, [M+H]⁺. ¹H NMR (400 MHz, DMSO-d₆) δ 9.90 (s, 1H), 9.54 (s, 1H), 9.33 (s, 1H), 8.73 (s, 1H), 8.34 (d, J=2.0 Hz, 1H), 8.31 (d, J=1.2 Hz, 1H), 8.14-8.09 (m, 1H), 7.76 (d, J=9.6 Hz, 1H), 7.72-7.64 (m, 2H), 7.46 (d, J=8.0 Hz, 1H), 6.96-6.88 (m, 1H), 3.85-3.78 (m, 4H), 3.21-3.14 (m, 4H), 3.05 (d, J=4.4 Hz, 3H).

### Example 537: Synthesis of N⁶-(3-fluoropyridin-2-yl)-M-methyl-4-(6-morpholino-[1,2,4]triazolo[1,5-a]pyridin-2-yl)-2,7-naphthyridine-1,6-diamine

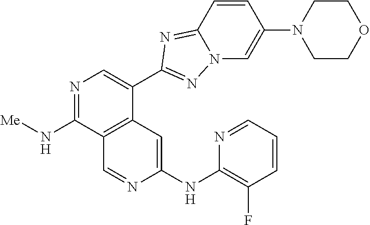 [SMILES: CNc1ncc(-c2nc3ccc(N4CCOCC4)cn3n2)c2cc(Nc3ncccc3F)ncc12]

N⁶-(3-fluoropyridin-2-yl)-N¹-methyl-4-(6-morpholino-[1,2,4]triazolo[1,5-a]pyridin-2-yl)-2,7-naphthyridine-1,6-diamine was synthesized using a similar procedure that was previously described in Example 536, by using N¹-methyl-4-(6-morpholino-[1,2,4]triazolo[1,5-a]pyridin-2-yl)-2,7-naphthyridine-1,6-diamine and 2-chloro-3-fluoropyridine as the starting material. LCMS (ESI) m/z 472.2, [M+H]⁺. ¹H NMR (400 MHz, DMSO-d₆) δ 9.58 (s, 1H), 9.34 (s, 1H), 9.28 (s, 1H), 8.78 (s, 1H), 8.33 (d, J=2.0 Hz, 1H), 8.21-8.13 (m, 2H), 7.75 (d, J=9.6 Hz, 1H), 7.71-7.65 (m, 2H), 7.11-7.01 (m, 1H), 3.84-3.77 (m, 4H), 3.21-3.14 (m, 4H), 3.06 (d, J=4.4 Hz, 3H).

### Example 538: Synthesis of 2-(6-((8-(methylamino)-5-(6-morpholino-[1,2,4]triazolo[1,5-a]pyridin-2-yl)-2,7-naphthyridin-3-yl)amino)pyridin-3-yl)propan-2-ol

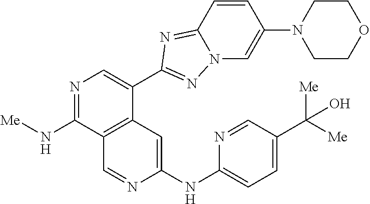 [SMILES: CNc1ncc(-c2nc3ccc(N4CCOCC4)cn3n2)c2cc(Nc3ccc(C(C)(C)O)cn3)ncc12]

A stirring mixture of N¹-methyl-4-(6-morpholino-[1,2,4]triazolo[1,5-a]pyridin-2-yl)-2,7-naphthyridine-1,6-diamine (80.0 mg; 0.213 mmol; 1.00 eq.), 2-(6-bromopyridin-3-yl)propan-2-ol (137.7 mg; 0.639 mmol; 3.00 eq.), EPhos Pd G₄ (19.5 mg; 0.021 mmol; 0.10 eq.), EPhos (11.3 mg; 0.021 mmol; 0.1.0 eq.) and Cs₇CO₃ (138.4 mg; 0.426 mmol; 2.00 eq.) in 1,4-dioxane (4.0 mL) was stirred at 100° C. for 2 hours under nitrogen atmosphere. The desired product was detected via LCMS. The resulting mixture was concentrated under vacuum. The residue was purified by flash chromatography on silica gel column using 0-1.2% of MeOH in CH₂Cl₂ as eluent to provide 2-(6-((8-(methylamino)-5-(6-morpholino-[1,2,4]triazolo[1,5-a]pyridin-2-yl)-2,7-naphthyridin-3-yl)amino)pyridin-3-yl)propan-2-ol as a yellow solid (28.9 mg, 25.4%). LCMS (ESI) m/z 512.2, [M+H]⁺. ¹H NMR (400 MHz, DMSO-d₆) δ 9.83 (s, 1H), 9.68 (s, 1H), 9.31 (s, 1H), 8.75 (s, 1H), 8.43 (d, J=2.4 Hz, 1H), 8.32 (d, J=1.6 Hz, 1H), 8.13-8.06 (m, 7.78-7.71 (m, 3H), 7.36 (d, J=8.8 Hz, 1H), 5.04 (s, 1H), 3.83-3.79 (m, 4H), 3.22-3.15 (m, 4H), 3.04 (d, J=4.4 Hz, 3H), 1.48 (s, 6H).

### Example 539: Synthesis of (1S,2R)-2-methyl-N-(8-(methylamino)-5-(6-((S)-2-1.0 methylmorpholino)-[1,2,4]triazolo[1,5-a]pyridin-2-yl)-2,7-naphthyridin-3-yl)cyclopropane-1-carboxamide

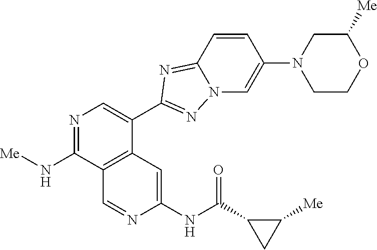 [SMILES: CNc1ncc(-c2nc3ccc(N4CCO[C@@H](C)C4)cn3n2)c2cc(NC(=O)[C@H]3C[C@H]3C)ncc12]

To a stirred mixture of (S)—N¹-methyl-4-(6-(2-methylmorpholino)-[1,2,4]triazolo[1,5-a]pyridin-2-yl)-2,7-naphthyridine-1,6-diamine (300.0 mg; 0.77 mmol; 1.00 eq.) and (1S,2R)-2-methylcyclopropane-1-carboxylic acid (69.2 mg; 0.69 mmol; 0.90 eq.) in pyridine (10 mL) was added POCl₃ (354.6 mg; 2.31 mmol; 3.00 eq.) dropwise at 0′C. The resulting mixture was stirred at room temperature for 1 hour under nitrogen atmosphere. The desired product was detected via LCMS. The mixture was diluted with EtOAc (100 mL) and washed with brine (3×5 mL). The organic layer was dried with Na₂SO₄, filtered and the solvent was removed under reduced pressure. The residue was purified by flash chromatography on silica gel column using 5-10% of MeOH in CH₂Cl₂ as eluent to afford the crude product. The crude product was purified by flash chromatography on pre-packed C18 column using 30-60% of MeOH in water (10 mmol/L NH₄HCO₃) as eluent to provide (1S,2R)-2-methyl-N-(8-(methylamino)-5-(6-(0)-2-methylmorpholino)-[1,2,4]triazolo[1,5-a]pyridin-2-yl)-2,7-naphthyridin-3-yl)cyclopropane-1-carboxamide as a yellow solid (215.6 mg, 58.8%). LCMS (ESI) m/z 473.2, [M+H]⁺. ¹H NMR (400 MHz, DMSO-d₆) δ 10.80 (s, 1H), 9.39 (s, 1H), 9.37 (s, 1H), 8.71 (s, 1H), 8.33 (d, J=2.0 Hz, 1H), 8.27-8.19 (m, 1H), 7.74 (d, J=9.6 Hz, 1H), 7.68 (dd, J=9.6, 2.0 Hz, 1H), 3.99-3.90 (m, 1H), 3.77-3.57 (m, 3H), 3.55-3.47 (m, 1H), 3.05 (d, J=4.4 Hz, 3H), 2.79-2.68 (m, 1H), 2.46-2.36 (m, 1H), 2.13-2.03 (m, 1H), 1.35-1.21 (m, 1H), 1.18 (d, J=6.0 Hz, 3H), 1.12 (d, J=6.0 Hz, 3H), 1.03-0.94 (m, 1H), 0.88-0.79 (m, 1H).

### Example 540: Synthesis of (S)-1-fluoro-N-(8-(methylamino)-5-(6-(2-methylmorpholino)-[1,2,4]triazolo[1,5-a]pyridin-2-yl)-2,7-naphthyridin-3-yl)cyclopropane-1-carboxamide

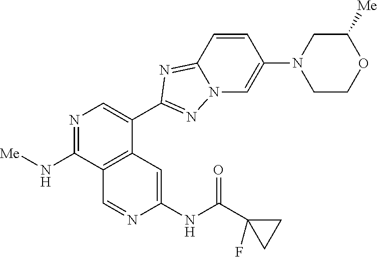 [SMILES: CNc1ncc(-c2nc3ccc(N4CCO[C@@H](C)C4)cn3n2)c2cc(NC(=O)C3(F)CC3)ncc12]

(S)-1-fluoro-N-(8-(methylamino)-5-(6-(2-methylmorpholino)-[1,2,4]triazolo[1,5-a]pyridin-2-yl)-2,7-naphthyridin-3-yl)cyclopropane-1-carboxamide was synthesized using a similar procedure that was previously described in Example 539 by using (S)—N¹-methyl-4-(6-(2-methylmorpholino)-[1,2,4]triazolo[1,5-a]pyridin-2-yl)-2,7-naphthyridine-1,6-diamine and 1-fluorocyclopropane-1-carboxylic acid as the starting material. LCMS (ESI) m/z 477.2, [M+H]⁺. ¹H NMR (400 MHz, DMSO-d₆) δ 10.32 (s, 1H), 9.46 (s, 1H), 9.43 (s, 1H), 8.79 (s, 1H), 8.42-8.28 (m, 2H), 7.75 (d, J=9.6 Hz, 1H), 7.69 (dd, J=9.6, 2.0 Hz, 1H), 4.00-3.89 (m, 1H), 3.78-3.58 (m, 3H), 3.55-3.48 (m, 1H), 3.07 (d, J=4.4 Hz, 3H), 2.79-2.65 (m, 1H), 2.48-2.37 (m, 1H), 1.52-1.38 (m, 4H), 1.18 (d, J=6.0 Hz, 3H).

### Example 541 and 542: Synthesis of (1S,2R)—N-(8-(methylamino)-5-(6-((S)-2-methylmorpholino)-[1,2,4]triazolo[1,5-a]pyridin-2-yl)-2,7-naphthyridin-3-yl)-2-(trifluoromethyl)cyclopropane-1-carboxamide (Example 541) and (1R,2S)—N-(8-(methylamino)-5-(6-((S)-2-methylmorpholino)-[1,2,4]triazolo[1,5-a]pyridin-2-yl)-2,7-naphthyridin-3-yl)-2-(trifluoromethyl)cyclopropane-1-carboxamide (Example 542)

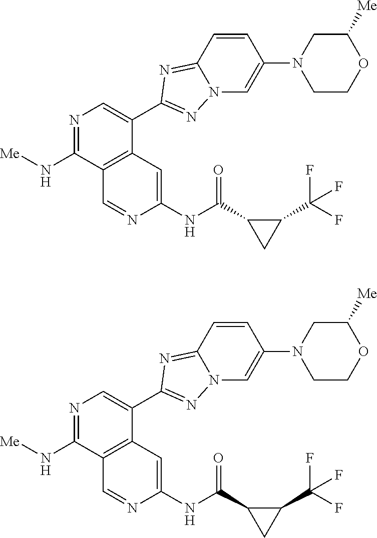 [SMILES: CNc1ncc(-c2nc3ccc(N4CCO[C@@H](C)C4)cn3n2)c2cc(NC(=O)[C@H]3C[C@H]3C(F)(F)F)ncc12 ; CNc1ncc(-c2nc3ccc(N4CCO[C@@H](C)C4)cn3n2)c2cc(NC(=O)[C@@H]3C[C@@H]3C(F)(F)F)ncc12]

To a stirred solution of (S)—N¹-methyl-4-(6-(2-methylmorpholino)-[1,2,4]triazolo[1,5-c]pyridin-2-yl)-2,7-naphthyridine-1,6-diamine (100.0 mg; 0.256 mmol; 1.00 eq.) and 2-(trifluoromethyl)cyclopropane-1-carboxylic acid (cis racemate) (36.0 mg; 0.234 mmol; 0.91 eq.) in pyridine (5 mL) was added POCl₃ (117.7 mg; 0.768 mmol; 100 eq.) dropwise at 0° C. The reaction was stirred at room temperature for 0.5 hour under nitrogen atmosphere. The desired product was detected via LCMS. The resulting mixture was diluted with EtOAc (90 mL), washed with brine (3×5 mL). The organic layer was dried over anhydrous Na₂SO₄, filtered, was concentrated under reduced pressure. The residue was purified by flash chromatography on silica gel column using 2-10% of MeOH in CH₂Cl₂ as eluent to provide the cis racemate. The cis racemate was separated by Chiral-HPLC (Column: CHIRAL ART Cellulose-SC, 2×25 cm, 5 μm; Mobile Phase A: Hex (0.5% 2 M NH₃-MeOH)-HPLC, Mobile Phase B: EtOH:CH₂Cl₂=1:1-HPLC; Flow rate: 20 mL/min; Gradient: 45% B to 45% B in 15.4 min) to afford (1S,2R)—N-(8-(methylamino)-5-(6-((S)-2-methylmorpholino)-[1,2,4]triazolo[1,5-a]pyridin-2-yl)-2,7-naphthyridin-3-yl)-2-(trifluoromethyl)cyclopropane-1-carboxamide (Example 541, the faster peak) as a yellow solid (15.3 mg, 11.3%) and (1R,2S)—N-(8-(methylamino)-5-(6-((S)-2-methylmorpholino)-[1,2,4]triazolo[1,5-a]pyridin-2-0)-2,7-naphthyridin-3-34)-2-(trifluoromethyl)cyclopropane-1-carboxamide (Example 542, the slower peak) as a yellow solid (19.2 mg, 14.2%). The two configurations are arbitrarily assigned. LCMS (ESI) m/z 527.2, [M+H]⁺. HNMR for Example 541: ¹H NMR (400 MHz, DMSO-d₆) δ 11.06 (s, 1H), 9.41 (s, 1H), 9.39 (s, 1H), 8.73 (s, 1H), 8.31 (d, J=2.0 Hz, 1H), 8.29-8.24 (m, 1H), 7.74 (d, J=9.6 Hz, 1H), 7.69 (dd, J=9.6, 2.0 Hz, 1H), 4.01-3.90 (m, 1H), 3.76-3.47 (m, 4H), 3.05 (d, J=4.4 Hz, 3H), 2.79-2.68 (m, 1H), 2.48-2.36 (m, 2H), 2.32-2.17 (m, 1H), 1.61-1.52 (m, 1H), 1.36-1.30 (m, 1H), 1.18 (d, J=6.4 Hz, 3H).

HNMR for Example 542: ¹H NMR (400 MHz, DMSO-d₆) δ 11.06 (s, 1H), 9.41 (s, 1H), 9.39 (s, 1H), 8.73 (s, 1.14), 8.31 (d, J=2.0 Hz, 1.111), 8.29-8.24 (m, 1H), 7.74 (d, J=9.6 Hz, 1H), 7.69 (dd, J=9.6, 2.0 Hz, 1H), 4.01-3.90 (m, 1H), 3.76-3.47 (m, 4H), 3.05 (d, J=4.4 Hz, 3H), 2.79-2.68 (m, 1H), 2.48-2.36 (m, 2H), 2.32-2.17 (m, 1H), 1.61-1.52 (m, 1H), 1.36-1.30 (m, 1H), 1.18 (d, J=6.4 Hz, 3H).

### Example 543: Synthesis of (S)—N¹-methyl-4-(6-(2-methylmorpholino)-[1,2,4]triazolo[1,5-a]pyridin-2-yl)-N⁶-(pyridin-2-yl)-2,7-naphthyridine-1,6-diamine

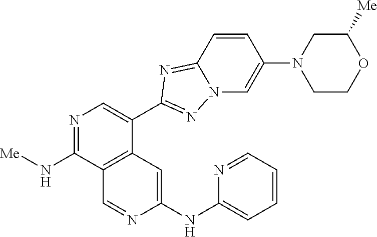 [SMILES: CNc1ncc(-c2nc3ccc(N4CCO[C@@H](C)C4)cn3n2)c2cc(Nc3ccccn3)ncc12]

A mixture of (S)—N¹-methyl-4-(6-(2-methylmorpholino)-[1,2,4]triazolo[1,5-a]pyridin-2-yl)-2,7-naphthyridine-1,6-diamine (180.0 mg; 0.461 mmol; 1.00 eq.), 2-chloropyridine (62.8 mg; 0.553 mmol; 1.20 eq.), Pd₂(dba)₃ (101.3 mg; 0.111 mmol; 0.24 eq.), XantPhos (128.0 mg; 0.221 mmol; 0.48 eq.) and Cs₂CO₃ (300.4 mg; 0.922 mmol; 2.00 eq.) in 1,4-dioxane (5 mL) was stirred at 130° C. for 3 hours under nitrogen atmosphere. Upon completion, the resulting mixture was concentrated under reduced pressure. The residue was purified by flash chromatography on silica gel column using 2-1.0% of MeOH in CH₂Cl₂ as eluent to afford the crude product. The crude product was purified by flash chromatography on pre-packed C18 column using 40-70% of MeOH in water (10 mmol/L NH₄HCO₃) as eluent to provide (S)—N¹-methyl-4-(6-(2-methylmorpholino)-[1,2,4]triazolo[1,5-a]pyridin-2-yl)-N⁶-(pyridin-2-yl)-2,7-naphthyridine-1,6-diamine as a yellow solid (31.2 mg, 12.9%). LCMS (ESI) m/z 468.2, NMR (400 MHz, DMSO-d₆) δ 10.01 (s, 1H), 9.53 (s, 1H), 9.36 (s, 1H), 8.69 (s, 1H), 8.40-8.24 (m, 3H), 7.77 (d, J=9.6 Hz, 1H), 7.74-7.66 (m, 2H), 7.48 (d, J=8.4 Hz, 1H), 6.99-6.89 (m, 1H), 4.00-3.92 (m, 1H), 3.79-3.59 (m, 3H), 3.59-3.48 (m, 1H), 3.06 (d, J=4.4 Hz, 3H), 2.80-2.79 (m, 1H), 2.49-2.39 (m, 1H), 1.18 (d, J=6.4 Hz, 3H).

### Example 544: Synthesis of (S)—N⁶-(1,5-dimethyl-1H-pyrazol-3-yl)-N¹-methyl-4-(6-(2-methylmorpholino)-[1,2,4]triazolo[1,5-a]pyridin-2-yl)-2,7-naphthyridine-1,6-diamine

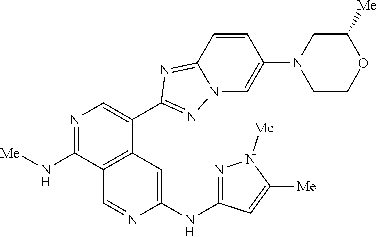 [SMILES: CNc1ncc(-c2nc3ccc(N4CCO[C@@H](C)C4)cn3n2)c2cc(Nc3cc(C)n(C)n3)ncc12]

To a stirred mixture of (S)—N¹-methyl-4-(6-(2-methylmorpholino)-[1,2,4]triazolo[1,5-a]pyridin-2-yl)-2,7-naphthyridine-1,6-diamine (60.0 mg; 0.154 mmol; 1.00 eq.) in 1,4-dioxane (3 mL) was added EPhos Pd G₄ (14.1 mg; 0.015 mmol; 0.10 eq.), EPhos (8.2 mg; 0.015 mmol; 0.10 eq.), Cs₂CO₃ (200.6 mg; 0.616 mmol; 4.01 eq.) and 3-bromo-1,5-dimethyl-1H-pyrazole (26.8 mg; 0.153 mmol; 1.00 eq.) at room temperature. The reaction was stirred at 120° C. for 3 hours under nitrogen atmosphere. After completion, the resulting mixture was cooled to RT and concentrated under vacuum. The residue was purified by flash chromatography on silica gel column using 2-10% of MeOH in CH₂Cl₂ as eluent to provide (S)—N⁶-(1,5-dimethyl-1H-pyrazol-3-yl)-N¹-methyl-4-(6-(2-methylmorpholino)-[1,2,4]triazolo[1,5-a]pyridin-2-yl)-2,7-naphthyridine-1,6-diamine as a yellow solid (34.3 mg, 46.0%). LCMS (ESI) m/z 485.2, [M+H]⁺. ¹H NMR (400 MHz, DMSO-d₆) δ 9.29 (s, 1H), 9.22 (s, 1H), 8.95 (s, 1H), 8.69 (s, 1H), 8.31 (d, J=2.0 Hz, 1H), 8.05-7.97 (m, 1H), 7.77-7.65 (m, 2H), 6.03 (s, 1H), 3.99-3.91 (m, 1H), 3.77-3.65 (m, 2H), 3.70 (s, 3H), 3.63-3.49 (m, 2H), 3.02 (d, J=4.4 Hz, 3H), 2.77-2.66 (m, 1H), 2.45-2.35 (m, 1H), 2.28 (5, 3H), 1.1.8 (d, J=6.4 Hz, 3H).

### Example 545: Synthesis of (S)—N¹-methyl-N⁶-(1-methyl-1H-pyrazol-3-yl)-4-(6-(2-methylmorpholino)-[1,2,4]triazolo[1,5-a]pyridin-2-yl)-2,7-naphthyridine-1,6-diamine

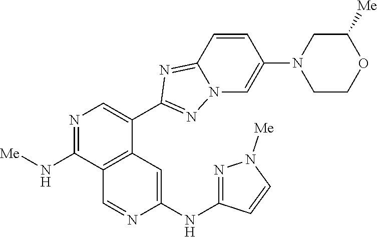 [SMILES: CNc1ncc(-c2nc3ccc(N4CCO[C@@H](C)C4)cn3n2)c2cc(Nc3ccn(C)n3)ncc12]

(S)—N¹-methyl-N⁶-(1-methyl-1H-pyrazol-3-yl)-4-(6-(2-methylmorpholino)-[1,2,4]triazolo[1,5-a]pyridin-2-yl)-2,7-naphthyridine-1,6-diamine was synthesized using a similar procedure that was previously described in Example 544 by using (S)—N¹-methyl-4-(6-(2-methylmorpholino)-[1,2,4]triazolo[1,5-a]pyridin-2-yl)-2,7-naphthyridine-1,6-diamine and 3-bromo-1-methyl-1H-pyrazole as the starting material. LCMS (ESI) m/z 471.2, [M+H]⁺. ¹H NMR (400 MHz, DMSO-d₆) δ 9.44 (s, 1H), 9.24 (s, 1H), 9.00 (s, 1H), 8.68 (s, 1H), 8.33 (d, J=2.0 Hz, 1H), 8.10-8.00 (m, 1H), 7.78-7.66 (m, 2H), 7.57 (d, J=2.4 Hz, 1H), 6.1.5 (d, J=2.4 Hz, 1H), 4.01-3.90 (m, 1H), 3.83 (s, 3H), 3.77-3.65 (m, 2H), 3.63-3.48 (m, 2H), 3.03 (d, J=4.4 Hz, 3H), 2.77-2.67 (m, 1H), 2.45-2.35 (m, 1H), 1.18 (d, J=6.4 Hz, 3H).

### Example 546: Synthesis of (1R,2S)-2-methyl 1N-(8-(methylamino)-5-(6-((R)-2-methylmorpholino)-[1,2,4]triazolo[1,5-a]pyridin-2-yl)-2,7-naphthyridin-3-yl)cyclopropane-1-carboxamide

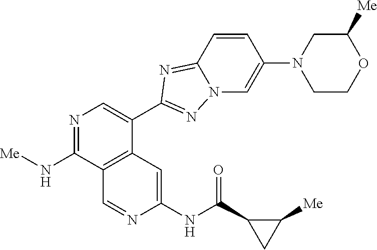 [SMILES: CNc1ncc(-c2nc3ccc(N4CCO[C@H](C)C4)cn3n2)c2cc(NC(=O)[C@@H]3C[C@@H]3C)ncc12]

To a stirred solution of (R)—N¹-methyl-4-(6-(2-methylmorpholino)-[1,2,4]triazolo[1,5-a]pyridin-2-yl)-2,7-naphthyridine-1,6-diamine (100.0 mg; 0.256 mmol; 1.00 eq.) and (1R,19-2-methylcyclopropane-1-carboxylic acid (23.0 mg; 0.230 mmol; 0.90 eq.) in pyridine (5 mL) was added POCl₃ (117.8 mg; 0.768 mmol; 3.00 eq.) dropwise at 0 GC. The resulting mixture was stirred at room temperature for 0.5 hour under nitrogen atmosphere. The desired product was detected via LCMS. The reaction solution was added to water (10 mL) and extracted with EtOAc (3×50 mL). The combined organic layers were washed with brine (3×5 mL), dried over Na₂SO₄, filtered, and concentrated under reduced pressure. The residue was purified by flash chromatography on silica gel column using 5-10% of MeOH in CH₂Cl₂ as eluent to afford the crude product. The crude product was purified by flash chromatography on pre-packed C18 column using 50-80% of MeOH in water (10 mmol/L NH₄HCO₃) as eluent to provide (1R,2S)-2-methyl-N-(8-(methyl amino)-5-(6-((R)-2-methyl morpholino)-[1,2,4]triazolo[1, 5-a]pyridin-2-yl)-2,7-naphthyridin-3-yl)cyclopropane-1-carboxamide as a yellow solid (47.8 mg, 39.8%). LCMS (ESI) m/z 473.2, [M+H]⁺. ¹H NMR (400 MHz, DMSO-d₆) δ 10.80 (s, 1H), 9.39 (s, 1H), 9.37 (s, 1H), 8.71 (s, 1H), 8.33 (d, J=2.0 Hz, 1H), 8.27-8.19 (m, 1H), 7.74 (d, J=9.6 Hz, 1H), 7.68 (dd, J=9.6, 2.0 Hz, 1H), 3.99-3.90 (m, 1H), 3.77-3.57 (m, 3H), 3.55-3.47 (m, 1H), 3.05 (d, J=4.4 Hz, 3H), 2.79-2.68 (m, 1H), 2.46-2.36 (m, 1H), 2.13-2.03 (m, 1H), 1.35-1.21 (m, 1H), 1.18 (d, J=6.0 Hz, 3H), 1.12 (d, J=6.0 Hz, 3H), 1.03-0.94 (m, 1H), 0.88-0.79 (m, 1H).

### Examples 547-550

Each compound in Table 23 below was prepared using a similar experimental procedure to prepare in Example 546, using (R) N¹-methyl-4-(6-(2-methylmorpholino)-[1,2,4]triazolo[1,5-a]pyridin-2-yl)-2,7-naphthyridine-1,6-diamine as the common intermediate and appropriate acid.

TABLE 23

| Example | Structure and Name | MS and ¹H NMR |
|---|---|---|
| Example 547 | 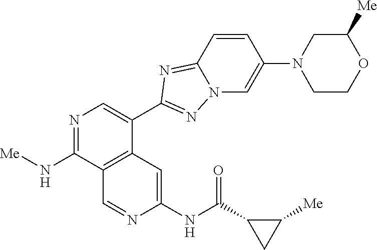 | LCMS (ESI) m/z 473.2, [M + H]⁺. ¹H NMR (400 MHz, DMSO-d₆) δ 10.80 (s, 1H), 9.39 (s, 1H), 9.37 (s, 1H), 8.70 (s, 1H), 8.34 (d, J = 2.0 Hz, 1H), 8.28- 8.20 (m, 1H), 7.74 (d, J = 9.6 Hz, 1H), 7.69 (dd, J = 9.6, 2.0 Hz, 1H), 4.00-3.91 (m, 1H), 3.79-3.58 (m, 3H), 3.52-3.47 (m, 1H), 3.05 (d, J = 4.4 Hz, 3H), 2.79-2.69 (m, 1H), 2.47- 2.38 (m, 1H), 2.12-2.02 (m, 1H), 1.36-1.23 (m, 1H), 1.18 (d, J = 6.0 Hz, 3H), 1.12 (d, J = 6.0 Hz, 3H), 1.02- 0.95 (m, 1H), 0.89-0.80 (m, 1H). |
| | (1S,2R)-2-methyl-N-(8- | |
| | (methylamino)-5-(6-((R)-2- | |
| | methylmorpholino)- | |
| | [1,2,4]triazolo[1,5-a]pyridin-2- | |
| | yl)-2,7-naphthyridin-3- | |
| | yl)cyclopropane-1-carboxamide | |
| Example 548 | 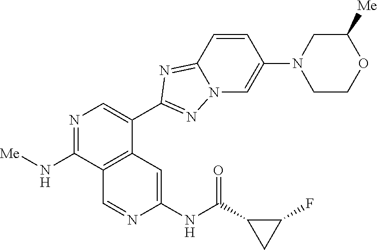 | LCMS (ESI) m/z 477.2, [M + H]⁺. ¹H NMR (400 MHz, DMSO-d₆) δ 10.97 (s, 1H), 9.41 (s, 1H), 9.40 (s, 1H), 8.73 (s, 1H), 8.33 (d, J = 2.0 Hz, 1H), 8.30- 8.22 (m, 1H), 7.75 (d, J = 9.6 Hz, 1H), 7.69 (d, J = 9.6, 2.0 Hz, 1H), 5.08- 4.83 (m, 1H), 4.00-3.92 (m, 1H), 3.78-3.58 (m, 3H), 3.53-3.48 (m, 1H), 3.06 (d, J = 4.4 Hz, 3H), 2.79- 2.69 (m, 1H), 2.45-2.18 (m, 1H), 2.33-2.22 (m, 1H), 1.77-1.63 (m, 1H), 1.29-1.19 (m, 1H), 1.18 (d, J = 6.0 Hz, 3H). |
| | (1R,2R)-2-fluoro-N-(8- | |
| | (methylamino)-5-(6-((R)-2- | |
| | methylmorpholino)- | |
| | [1,2,4]triazolo[1,5-a]pyridin-2- | |
| | yl)-2,7-naphthyridin-3- | |
| | yl)cyclopropane-1-carboxamide | |
| Example 549 | 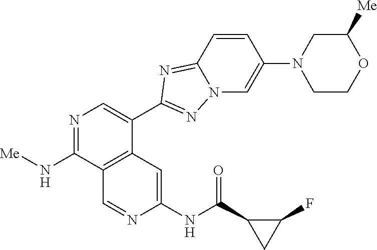 | LCMS (ESI) m/z 477.2 [M + H]⁺. ¹H NMR (400 MHz, DMSO-d₆) δ 10.97 (s, 1H), 9.41 (s, 1H), 9.40 (s, 1H), 8.73 (s, 1H), 8.33 (d, J = 2.0 Hz, 1H), 8.30- 8.22 (m, 1H), 7.75 (d, J = 9.6 Hz, 1H), 7.69 (d, J = 9.6, 2.0 Hz, 1H), 5.08- 4.83 (m, 1H), 4.00-3.92 (m, 1H), 3.78-3.58 (m, 3H), 3.53-3.48 (m, 1H), 3.06 (d, J = 4.4 Hz, 3H), 2.79- 2.69 (m, 1H), 2.45-2.18 (m, 1H), 2.33-2.22 (m, 1H), 1.77-1.63 (m, 1H), 1.29-1.19 (m, 1H), 1.18 (d, J = 6.0 Hz, 3H). |
| | (1S,2S)-2-fluoro-N-(8- | |
| | (methylamino)-5-(6-((R)-2- | |
| | methylmorpholino)- | |
| | [1,2,4]triazolo[1,5-a]pyridin-2- | |
| | yl)-2,7-naphthyridin-3- | |
| | yl)cyclopropane-1-carboxamide | |
| Example 550 | 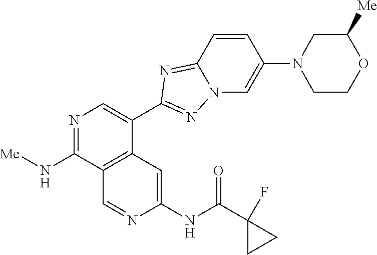 | LCMS (ESI) m/z 477.2, [M + H]⁺. ¹H NMR (400 MHz, DMSO-d₆) δ 10.32 (s, 1H), 9.46 (s, 1H), 9.43 (s, 1H), 8.80 (s, 1H), 8.38-8.31 (m, 2H), 7.75 (d, J = 9.6 Hz, 1H), 7.69 (dd, J = 9.6, 2.0 Hz, 1H), 3.98-3.91 (m, 1H), 3.76- 3.59 (m, 3H), 3.55-3.46 (m, 1H), 3.07 (d, J = 4.4 Hz, 3H), 2.79-2.68 (m, 1H), 2.46-2.36 (m, 1H), 1.54- 1.35 (m, 4H), 1.18 (d, J = 6.0 Hz, 3H). |
| | (R)-1-fluoro-N-(8-(methylamino)- | |
| | 5-(6-(2-methylmorpholino)- | |
| | [1,2,4]triazolo[1,5-a]pyridin-2- | |
| | yl)-2,7-naphthyridin-3- | |
| | yl)cyclopropane-1-carboxamide | |

### Example 551: Synthesis of (R)—N¹-methyl-4-(6-(2-methylmorpholino)[1,2,4]triazolo[1,5-a]pyridin-2-yl)-N⁶-(pyridin-2-yl)-2,7-naphthyridine-1,6-diamine

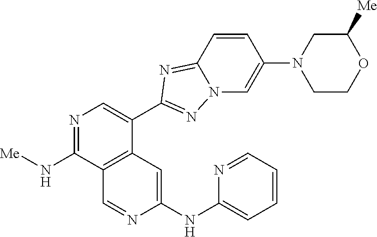 [SMILES: CNc1ncc(-c2nc3ccc(N4CCO[C@H](C)C4)cn3n2)c2cc(Nc3ccccn3)ncc12]

A mixture of (R)-M-methyl-4-(6-(2-methylmorpholino)-[1,2,4]triazolo[1,5-a]pyridin-2-yl)-2,7-naphthyridine-1,6-diamine (80.0 mg; 0.205 mmol; 1.00 eq.), 2-chloropyridine (23.1 mg; 0.205 mmol; 1.00 eq.), Pd₂(dba)₃ (45.0 mg; 0.049 mmol; 0.24 eq.), XantPhos (56.9 mg; 0.098 mmol; 0.48 eq.) and Cs₂CO₃ (133.5 mg; 0.410 mmol; 2.00 eq.) in 1,4-dioxane (2 mL) was stirred at 130° C. overnight under nitrogen atmosphere. LC-MS indicated that the reaction was complete. The resulting mixture was concentrated under reduced pressure. The residue was purified by flash chromatography, on silica gel column, using 2-1.0% of MeOH in CH₂Cl₂ as eluent to afford the crude product. The crude product was purified by flash chromatography on pre-packed C18 column using 50-80% of MeOH in water (10 mmol/L NH₄HCO₃) as eluent to provide (R)—N¹-methyl-4-(6-(2-methylmorpholino)-[1,2,4]triazolo[1,5-a]pyridin-2-yl)-N⁶-(pyridin-2-yl)-2,7-naphthyridine-1,6-diamine as a yellow solid (29.0 mg, 28.9%). LCMS (ESI) m/z 468.2, [M+H]⁺. ¹H NMR (400 MHz, DMSO-d₆) δ 9.88 (s, 1H), 9.52 (s, 1H), 9.33 (s, 1H), 8.73 (s, 1H), 8.35-8.27 (m, 2H), 8.15-8.08 (m, 1H), 7.76 (d, J=9.6 Hz, 1H), 7.73-7.64 (m, 2H), 7.51-7.44 (m, 1H), 6.98-6.88 (m, 1H), 4.01-3.91 (m, 1H), 3.79-3.59 (m, 3H), 3.55-3.48 (m, 1H), 3.05 (d, J=4.4 Hz, 3H), 2.80-2.72 (m, 1H), 2.46-2.40 (m, 1H), 1.18 (d, J=6.0 Hz, 3H).

### Example 552: Synthesis of (R)-1-cyclopropyl-3-(8-(methylamino)-5-(6-(2-methylmorpholino)-[1,2,4]triazolo[1,5-a]pyridin-2-yl)-2,7-naphthyridin-3-yl)urea

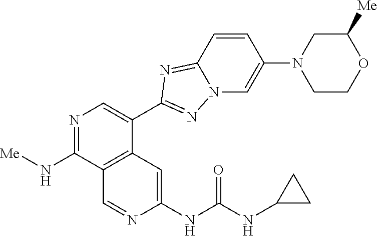

A solution of (R)—N¹-methyl-4-(6-(2-methyl morpholino)-[1,2,4]triazolo[1,5-a]pyridin-2-yl)-2,7-naphthyridine-1,6-diamine (100.0 mg; 0.256 mmol; 1.00 eq.) and isocyanatocyclopropane (106.4 mg; 1.280 mmol; 5.00 eq.) in THF (5 mL) was stirred at 60° C. overnight under nitrogen atmosphere. The desired product was detected via LCMS. The resulting mixture was concentrated under reduced pressure. The residue was purified by flash chromatography, on silica gel, column using 5-10% of MeOH in CH₂Cl₂ as eluent to afford (R)-1-cyclopropyl-3-(8-(methylamino)-5-(6-(2-methylmorpholino)-[1,2,4]triazolo[1, 5-a]pyridin-2-yl)-2,7-naphthyridin-3-yl)urea as a yellow solid (29.7 mg, 23.6%). LCMS (ESI) m/z 474.2, [M+H]⁺. ¹H NMR (400 MHz, DMSO-d₆) δ 9.29 (s, 1H), 9.13 (s, 1H), 8.95 (s, 1H), 8.68 (s, 1H), 8.33 (d, J=2.0 Hz, 1H), 8.20-8.10 (m, 1H), 7.75 (d, J=9.6 Hz, 1H), 7.69 (dd, J=9.6, 2.0 Hz, 1H), 7.55 (s, 1H), 3.99-3.91 (m, 1H), 3.78-3.58 (m, 3H), 3.55-3.48 (m, 1H), 3.04 (d, J=4.4 Hz, 3H), 2.79-2.58 (m, 2H), 2.47-2.37 (m, 1H), 1.18 (d, J=6.0 Hz, 3H), 0.73-0.62 (m, 2H), 0.49-0.41 (m, 2H).

### Example 553: Synthesis of 4-(6-((2R,6S)-2,6-dimethylmorpholino)-[1,2,4]triazolo[1,5-a]pyridin-2-yl)-N¹-methyl-2,7-naphthyridine-1,6-diamine

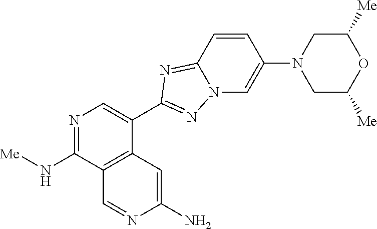

To a solution of N-(5-(6-((2R,6S)-2,6-dimethylmorpholino)-[1,2,4]triazolo[1,5-a]pyridin-2-yl)-8-(methyl amino)-2,7-naphthyridin-3-yl)cyclopropanecarboxamide (Example 479) (474.0 mg; 1.003 mmol; 1.00 eq.) in MeOH (30 mL) was added a solution of NaOH (401.2 mg; 10.031 mmol; 10.00 eq.) in water (9 mL) at 0° C. The resulting mixture was stirred at 60° C. overnight. The desired product was detected via LCMS. The reaction was allowed to cool to room temperature, the precipitated solids were collected by filtration and washed with water (3×5 mL). The solids were dried under vacuum to afford 4-(6-((2R,6S)-2,6-dimethylmorpholino)-[1,2,4]triazolo[1,5-a]pyridin-2-yl)-N¹-methyl-2,7-naphthyridine-1,6-diamine as a light yellow solid (364.0 mg, 89.1%). 10 mg of the desired product was further purified by flash chromatography on pre-packed C18 column using 20-60% of MeOH in water (10 mmol/L NH₄HCO₃) as eluent to provide 4-(6-((2R,6S)-2,6-dimethylmorpholino)-[1,2,4]triazolo[1,5-a]pyridin-2-yl)-N¹-methyl-2,7-naphthyridine-1,6-diamine as a light yellow solid. (2.8 mg, 28.0%). LCMS (ESI) m/z 405.2, [M+H]⁺.

### Example 554: Synthesis of (1S,2S)—N-(5-(6-((2R,6S)-2,6-dimethylmorpholino)-[1,2,4]triazolo[1,5-a]pyridin-2-yl)-8-(methylamino)-2,7-naphthyridin-3-yl)-2-fluoracyclopropane-1-carboxamide

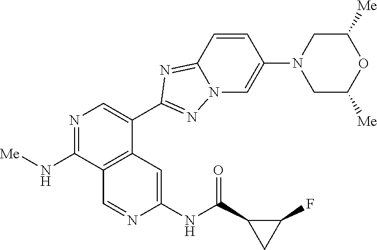

To a stirred solution of 4-(6-((2R,6S)-2,6-dimethylmorpholino)-[1,2,4]triazolo[1,5-a]pyridin-2-yl)-N¹-methyl-2,7-naphthyridine-1,6-diamine (Example 553) (100.0 mg; 0.247 mmol; 1.00 eq.) and (1S,2S)-2-fluorocyclopropane-1-carboxylic acid (23.0 mg; 0.221 mmol; 0.89 eq.) in pyridine (10 mL) was added POCl₃ (113.6 mg; 0.741 mmol; 3.00 eq.) dropwise at 0° C. The reaction was stirred at room temperature for 0.5 hour under nitrogen atmosphere. The desired product was detected via LCMS. The mixture was diluted with EtOAc (90 mL) and washed with brine (3×8 mL). The organic layer was dried over Na₂SO₄ and the solvent was removed under reduced pressure. The residue was purified by flash chromatography on silica gel column using 2-10% of MeOH in CH₂Cl₂ as eluent to provide (1S,2S) N-(5-(6-((2R,6S)-2,6-dimethylmorpholino)-[1,2,4]triazido[1,5-a]pyridin-2-yl)-8-(methylamino)-2,7-naphthyridin-3-yl)-2-fluorocyclopropane-1-carboxamide as a yellow solid (32.6 mg, 26.8%). LCMS (ESI) m/z 491.2, [M+H]⁺. ¹H NMR (400 MHz, DMSO-d₆) δ 10.97 (s, 1H), 9.41 (s, 1H), 9.39 (s, 1H), 8.72 (s, 1H), 8.33 (d, J=2.0 Hz, 1H), 8.30-8.22 (m, 1H), 7.79-7.67 (m, 2H), 5.08-4.82 (m, 1H), 3.82-3.71 (m, 2H), 3.65-3.58 (m, 2H), 3.05 (d, J=4.4 Hz, 3H), 2.37-2.22 (m, 3H), 1.76-1.61 (m, 1H), 1.25-1.11 (m, 7H).

### Examples 555 and 556

Each compound in Table 24 below was prepared using a similar experimental procedure to prepare in Example 554, using 4-(6-((2R,6S)-2,6-di methylmorpholino)-[1,2,4]triazolo[1,5-a]pyridin-2-yl)-N¹-methyl-2,7-naphthyridine-1,6-diamine as the common intermediate and appropriate acid.

| Example | Structure and Name | MS and ¹H NMR |
|---|---|---|
| Example 555 | 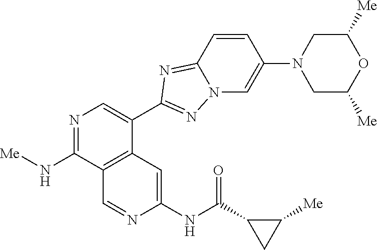 | LCMS (ESI) m/z 487.2, [M + H]⁺. ¹H NMR (400 MHz, DMSO-d₆) δ 10.78 (s, 1H), 9.39 (s, 1H), 9.36 (s, 1H), 8.70 (s, 1H), 8.33 (d, J = 2.0 Hz, 1H), 8.25- 8.19 (m, 1H), 7.74 (d, J = 9.6 Hz, 1H), 7.68 (dd, J = 9.6, 2.0 Hz, 1H), 3.80-3.70 (m, 2H), 3.65-3.55 (m, 2H), 3.05 (d, J = 4.4 Hz, 3H), 2.36- 2.31 (m, 2H), 2.10-2.05 (m, 1H), 1.32-1.23 (m, 1H), 1.18 (d, J = 6.4 Hz, 6H), 1.12 (d, J = 6.0 Hz, 3H), 1.01- 0.96 (m, 1H), 0.86-0.80 (m, 1H). |
| | (1S,2R)-N-(5-(6-((2R,6S)-2,6- | |
| | dimethylmorpholino)- | |
| | [1,2,4]triazolo[1,5-a]pyridin-2- | |
| | yl)-8-(methylamino)-2,7- | |
| | naphthyridin-3-yl)-2- | |
| | methylcyclopropane-1- | |
| | carboxamide | |
| Example 556 | 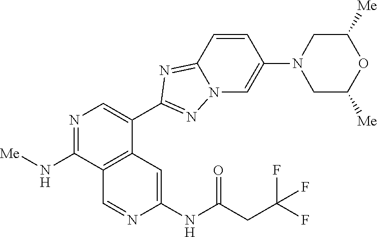 | LCMS (ESI) m/z 515.2, [M + H]⁺. ¹H NMR (400 MHz, DMSO-d₆) δ 11.31 (s, 1H), 10.10-9.90 (m, 1H), 9.62 (s, 1H), 9.58 (s, 1H), 8.47 (s, 1H), 8.35 (d, J = 2.0 Hz, 1H), 7.81 (d, J = 9.6 Hz, 1H), 7.76 (dd, J = 9.6, 2.0 Hz, 1H), 3.78-3.61 (m, 6H), 3.17 (d, J = 4,4 Hz, 3H), 2.38-2.32 (m, 2H), 1.18 (d, J = 6.0 Hz, 6H). |
| | N-(5-(6-((2R,6S)-2,6- | |
| | dimethylmorpholino)- | |
| | [1,2,4]triazolo[1,5-a]pyridin-2- | |
| | yl)-8-(methylamino)-2,7- | |
| | naphthyridin-3-yl)-3,3,3- | |
| | trifluoropropanamide | |

### Example 557: Synthesis of 4-([1,2,4]triazolo[1,5-a]pyridin-2-yl)-N¹-methyl-N⁶-(pyridin-2-yl)-2,7-naphthyridine-1,6-diamine

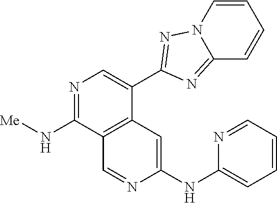 [SMILES: CNc1ncc(-c2nc3ccccn3n2)c2cc(Nc3ccccn3)ncc12]

### Step 1: 2-chloro-[1,2,4]triazolo[1,5-a]pyridine

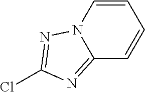

A stirred mixture of [1,2,4]triazolo[1,5-a]pyridin-2-amine (1.50 g; 11.18 mmol; 1.00 eq.) and CuCl₂ (0.45 g; 3.36 mmol; 0.30 eq.) in HCl (12 M in water, 24.8 mL) was stirred at 0° C. for 5 minutes. To the above mixture was added a solution of NaNO₂ (0.93 g; 13.418 mmol; 1.20 eq.) in water (5.3 mL) dropwise at 0° C. The resulting mixture was stirred at 0° C. for 0.5 hour and at room temperature for additional 3 hours. The desired product was detected via LCMS. The resulting mixture was diluted with water (50 mL). The mixture was neutralized to pH=7 with saturated NaHCO₃ solution. The resulting mixture was extracted with EtOAc (3×30 mL). The combined organic layers were washed with brine (3×10 mL), dried over anhydrous Na₂SO₄. After filtration, the filtrate was concentrated under reduced pressure to provide 2-chloro-[1,2,4]triazolo[1,5-a]pyridine as a white solid (1.68 g, 97.8%). LCMS (ESI) m/z 154.0, [M+H]⁺.

### Step 2: N-(5-([1,2,4]triazolo[1,5-a]pyridin-2-yl)-8-(methylamino)-2,7-naphthyridin-3-yl)cyclopropanecarboxamide

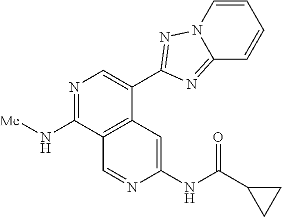 [SMILES: CNc1ncc(-c2nc3ccccn3n2)c2cc(NC(=O)C3CC3)ncc12]

To a stirring mixture of XPhos Pd G₃ (420.0 mg; 0.50 mmol; 0.20 eq.), XPhos (235.0 mg; 0.49 mmol; 0.20 eq.) and K₃PO₄ (1.55 g; 7.30 mmol; 2.99 eq.) in a mixture solvent of 1,4-dioxane/water (5:1, 12 mL) was added N=(8-(methylamino)-5-(4,4,5,5-tetramethyl-1,3,2-dioxaborolan-2-yl)-2,7-naphthyridin-3-yl)cyclopropanecarboxamide (900.0 mg; 2.44 mmol; 1.00 eq.) and 2-chloro-[1,2,4]triazolo[1,5-a]pyridine (375.0 mg; 2.44 mmol; 1.00 eq.) under nitrogen atmosphere. The resulting solution was stirred at 90° C. for 3 hours. The desired product was detected via LCMS. This reaction was cooled to RT and concentrated under vacuum. The residue was purified by flash chromatography on silica gel column using 2-10% of MeOH in CH₂Cl₂ as eluent to provide N-(5-([1,2,4]triazolo[1,5-a]pyridin-2-yl)-8-(methylamino)-2,7-naphthyridin-3-yl)cyclopropanecarboxamide as a brown solid (300.0 mg, 34.2%). LCMS (ESI) m/z 360.1, [M+H]⁺.

### Step 3: 4-([1,2,4]triazolo[1,5-a]pyridin-2-yl)-N¹-methyl-2,7-naphthyridine-1,6-diamine

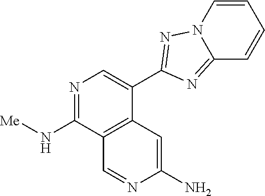 [SMILES: CNc1ncc(-c2nc3ccccn3n2)c2cc(N)ncc12]

To a stirred solution of N-(5-([1,2,4]triazolo[1,5-a]pyridin-2-yl)-8-(methylamino)-2,7-naphthyridin-3-yl)cyclopropanecarboxamide (280.0 mg; 0.640 mmol; 1.00 eq.) in DMSO/MeOH (1:4, 15 mL) was added a solution of NaOH (270.0 mg; 6.751 mmol; 9.58 eq.) in water (4 mL) at 0° C. The resulting mixture was stirred at 60° C. for 4 hours. Upon completion, this reaction mixture was concentrated under vacuum. The residue was purified by flash chromatography on pre-packed C18 column using 50-70% of MeCN in water (10 mmol/L NH₄HCO₃) as eluent to provide 4-([1,2,4]triazolo[1,5-a]pyridin-2-yl)-N¹-methyl-2,7-naphthyridine-1,6-diamine as a yellow solid (200.0 mg, 87.7%). LCMS (ESI) m/z 292.1, [M+H]⁺.

### Step 4: 4-([1,2,4]triazolo[1,5-a]pyridin-2-yl)-N¹-methyl-N⁶-(pyridin-2-yl)-2,7-naphthyridine-1,6-diamine

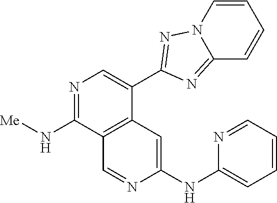 [SMILES: CNc1ncc(-c2nc3ccccn3n2)c2cc(Nc3ccccn3)ncc12]

To a stirring mixture of Pd₂(dba)₃ (38.0 mg; 0.04 mmol; 0.12 eq.), Xantphos (48.0 mg; 0.083 mmol; 0.24 eq.) and Cs₂CO₃ (225.0 mg; 0.69 mmol; 2.01 eq.) in 1,4-dioxane (7 mL) was added 2-chloropyridine (39.0 mg; 0.34 mmol; 1.00 eq.) and 4-([1,2,4]triazolo[1,5-a]pyridin-2-yl)-N¹-methyl-2,7-naphthyridine-1,6-diamine (100.0 mg; 0.34 mmol; 1.00 eq.) under nitrogen atmosphere. The resulting solution was stirred at 130° C. for 4 hours. The desired product was detected via LCMS. The reaction was concentrated under vacuum. The residue was purified by flash chromatography on silica gel column using 2-10% of MeOH in CH₂Cl₂ as eluent to afford 4-([1,2,4]triazolo[1,5-a]pyridin-2-yl)-N′-methyl N′-(pyridin-2-yl)-2,7-naphthyridine-1,6-diamine as a light yellow solid (41.6 mg, 32.9%). LCMS (ESI) m/z 369.1, [M+H]⁺. ¹H NMR (400 MHz, DMSO-d₆) δ 9.94 (s, 1H), 9.70 (s, 1H), 9.35 (s, 1H), 8.97 (d, J=6.8 Hz, 1H), 8.85 (s, 1H), 8.43-8.33 (m, 1H), 8.21-8.15 (m, 1H), 7.87 (d, J=8.8 Hz, 1H), 7.73-7.68 (m, 2H), 7.41 (d, J=8.4 Hz, 1H), 7.23-7.18 (m, 1H), 6.96-6.90 (m, 1H), 3.06 (d, J=4.4 Hz, 3H).

### Example 558: Synthesis of (1S,2R)—N-(5-(6-(4,4-difluoropiperidin-1-yl)-[1,2,4]triazolo[1,5-a]pyridin-2-yl)-8-(methylamino)-2,7-naphthyridin-3-yl)-2-methylcyclopropane-1-carboxamide

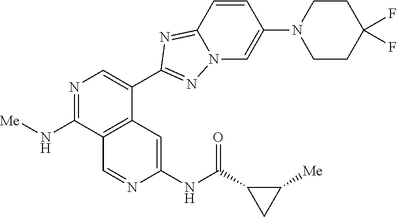

### Step 1: 4-(6-(4,4-difluoropiperidin-1-yl)-[1,2,4]triazolo[1,5-a]pyridin-2-yl)-N¹-methyl-2,7-naphthyridine-1,6-diamine

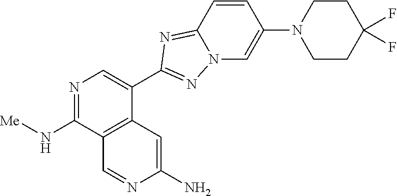

To a solution of N-(5-(6-(4,4-difluoropiperidin-1-yl)-[1,2,4]triazolo[1,5-a]pyridin-2-yl)-8-(methylamino)-2,7-naphthyridin-3-yl)cyclopropanecarboxamide (Example 483) (145.0 mg; 0.303 mmol; 1.00 eq.) in MeOH (14 mL) was added a solution of NaOH (121.2 mg; 3.030 mmol; 1.0.00 eq.) in water. (2 mL) at 0° C. The resulting mixture was stirred at 60° C. overnight.

The desired product was detected via LCMS. The reaction mixture was cooled to RT and the solvent was concentrated under vacuum. The residue was purified by flash chromatography on pre-packed C18 column using 20-95% of MeOH in water (10 mmol/L NH₄HCO₃) as eluent to provide 4-(6-(4,4-difluoropiperidin-1-yl)-[1,2,4]triazolo[1,5-a]pyridin-2-yl)-N¹-methyl-2,7-naphthyridine-1,6-diamine as a yellow solid (80.0 mg, 61.1%). LCMS (ESI) m/z 411.2, [M+H]⁺.

### Step 2: (1S,2R)—N-(5-(6-(4,4-difluoropiperidin-1-yl)[1,2,4]triazolo[1,5-a]pyridin-2-yl)-8-(methylamino)-2,7-naphthyridin-3-yl)-2-methylcyclopropane-1-carboxamide

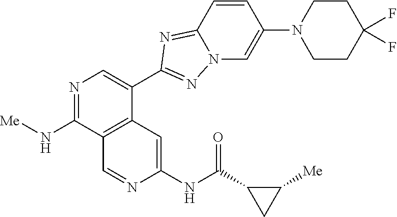

To a stirred solution of 4-(6-(4,4-difluoropiperidin-1-yl)-[1,2,4]triazolo[1,5-a]pyridin-2-yl)-N¹-methyl-2,7-naphthyridine-1,6-diamine (75.0 mg; 0.183 mmol; 1.00 eq.) and (1S,2R)-2-methylcyclopropane-1-carboxylic acid (16.5 mg; 0.165 mmol; 0.90 eq.) in pyridine (5 mL) was added POCl₃ (84.0 mg; 0.548 mmol; 3.00 eq.) dropwise at 0° C. The resulting mixture was stirred at room temperature for 0.5 hour under nitrogen atmosphere. LC-MS indicated that the reaction was complete. The resulting mixture was diluted with EtOAc (100 mL), washed with brine (2×10 mL), dried over anhydrous Na₂SO₄, filtered, and concentrated under reduced pressure. The residue was purified by flash chromatography on silica gel column using 2-1.5% of MeOH in CH₂Cl₂ as eluent to afford (1S,2R)—N-(5-(6-(4,4-difluoropiperidin-1-yl)-[1,2,4]triazolo[1,5-a]pyridin-2-yl)-8-(methyl amino)-2,7-naphthyridin-3-yl)-2-methylcyclopropane-1-carboxamide as a light yellow solid (29.8 mg, 32.7%). LCMS (ESI) m/z 493.2, [M+H]⁺. ¹H NMR (400 MHz, DMSO-d₆) δ 10.80 (s, 1H), 9.39 (s, 1H), 9.36 (s, 1H), 8.69 (s, 1H), 8.46 (d, J=2.0 Hz, 1H), 8.26-8.21 (m, 1H), 7.75 (d, J=9.6 Hz, 1H), 7.70 (dd, J=9.6, 2.0 Hz, 1H), 3.36-3.32 (m, 4H), 3.04 (d, J=4.4 Hz, 3H), 2.19-2.04 (m, 5H), 1.32-1.27 (m, 1H), 1.12 (d, J=6.0 Hz, 3H), 1.02-0.95 (m, 1H), 0.86-0.80 (m, 1H).

### Example 559: Synthesis of 1-fluoro-N-(5-(6-(7-methyl-9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropane-1-carboxamide

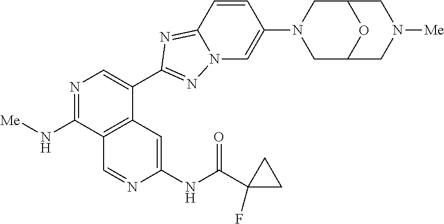

### Step 1: N¹-methyl-4-(6-(7-methyl-9-oxa-3,7-diazabicyclo[3.3.1]nonan-3-yl)-[1,2,4]triazolo[1,5-a]pyridin-2-yl)-2,7-naphthyridine-1,6-diamine

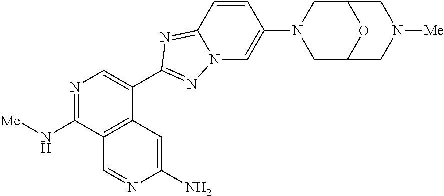

To a solution of N-(5-(6-(7-methyl-9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide (Example 504) (200.0 mg; 0.400 mmol; 1.00 eq.) in a mixture solvent of DMSO/MeOH (1:5, 6 mL) was added a solution of NaOH (160.1 mg; 4.003 mmol; 10.00 eq.) in water (1.5 mL) at 0° C. The mixture was stirred at 70° C. overnight. The desired product was detected via LCMS. The precipitated product was collected by filtration and washed with water (3×5 mL). The solids were dried under vacuum to afford N¹-methyl-4-(6-(7-methyl-9-oxa-3,7-diazabicyclo[3.3.1]nonan-3-yl)[1,2,4]triazolo[1,5-a]pyridin-2-yl)-2,7-naphthyridine-1,6-diamine as a yellow solid (90.0 mg, 52.2%). LCMS (I m/z 432.2, [M+H]⁺.

### Step 2: 1-fluoro-N-(5-(6-(7-methyl-9-oxa-3,7-diazabicyclo[3.3.1]nonan-3-yl)[1,2,4]triazolo[1,5-a]pyridin-2-yl)-8-(methylamino)-2,7-naphthyridin-3-yl)cyclopropane-1-carboxamide

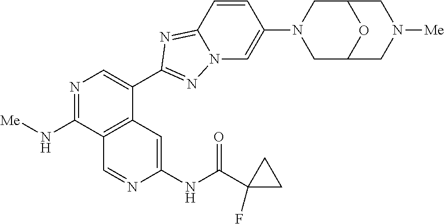

To a stirred solution of N¹-methyl-4-(6-(7-methyl-9-oxa-3,7-diazabicyclo[3.3.1]nonan-3-yl)-[1,2,4]triazolo[1,5-a]pyridin-2-yl)-2,7-naphthyridine-1,6-diamine (80.0 mg; 0.19 mmol; 1.00 eq.) and 1-fluorocyclopropane-1-carboxylic acid (17.3 mg; 0.166 mmol, 0.90 eq.) in pyridine (4 mL) was added POCl₃ (85.5 mg; 0.56 mmol; 3.01 eq.) dropwise at 0° C. The mixture was stirred at room temperature for 10 minutes under nitrogen atmosphere. The desired product was detected via LCMS. The reaction solution was diluted with EtOAc (50 mL), washed with water (2×5 The organic layer was concentrated under reduced pressure. The residue was purified by flash chromatography on silica gel column using 10-20% of MeOH in CH₂Cl₂ (0.1% Et₃N) as eluent to provide the crude product. The crude product was dissolved in DMSO (1.5 mL), and added dropwise into water (25 mL), whereupon precipitated product was isolated via filtration and washed with water (2×5 mL) to provide 1-fluoro-N-(5-(6-(7-methyl-9-oxa-3,7-diazabicyclo[3.3.1]nonan-3-yl)[1,2,4]triazolo[1,5-a]pyridin-2-yl)-8-(methy amino)-2,7-naphthyridin-3-yl)cyclopropane-1-carboxamide as an off-white solid (29.2 mg, 30.4%). LCMS (ESI) m/z 518.2, [M+H]⁺. ¹H NMR (400 MHz, DMSO-d₆) δ 1.0.30 (s, 1H), 9.48 (s, 1H), 9.46 (s, 1H), 8.82 (s, 1H), 8.34-8.30 (m, 8.16 (d, J=2.0 Hz, 1H), 7.72 (d, J=9.6 Hz, 1H), 7.62 (d, J=9.6, 2.0 Hz, 1H), 4.10-4.00 (m, 2H), 3.71-3.65 (m, 2H), 3.14-3.08 (m, 2H), 3.07 (d, J=4.4 Hz, 3H), 2.92-2.85 (m, 2H), 2.38-2.28 (m, 2H) 2.08 (s, 3H), 1.54-1.39 (m, 41-1).

### Example 560: Synthesis of (1S,2R)-2-methyl-N-(5-(6-(7-methyl-9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropane-1-carboxamide

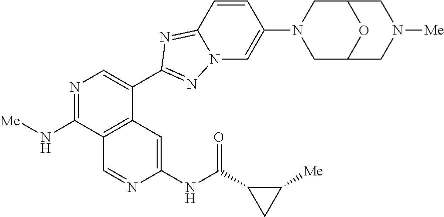

(1S,2R)-2-methyl-N-(5-(6-(7-methyl-9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropane-1-carboxamide was synthesized using a similar procedure that was previously described in Example 559, by using N¹-methyl-4-(6-(7-methyl-9-oxa-3,7-diazabicyclo[3.3.1]nonan-3-yl)-[1,2,4]triazolo[1,5-a]pyridin-2-yl)-2,7-naphthyridine-1,6-diamine (Example 559, Step 1) and (1S,2R)-2-methylcyclopropane-1-carboxylic acid as the starting material. LCMS (ESI) m/z 514.3, [M+H]⁺. ¹H NMR (400 MHz, DMSO-d₆) δ 10.80 (s, 1H), 9.41 (s, 1H), 9.39 (s, 1H), 8.73 (s, 1H), 8.27-8.15 (m, 2H), 7.75 (d, J=9.6 Hz, 1H), 7.65 (d, J=9.6, 2.0 Hz, 1H), 4.1.5-4.03 (m, 2H), 3.72-3.63 (m, 2H), 3.16-3.07 (m, 2H), 3.05 (d, J=4.4 Hz, 3H), 3.00-2.86 (m, 2H), 2.23-2.01 (m, 6H), 1.34-1.22 (m, 1H), 1.13 (d, J=6.0 Hz, 3H), 1.03-0.94 (m, 1H), 0.86-0.79 (m, 1H).

### Example 561: Synthesis of N-(5-(6-methyl-[1,2,4]triazolo[1,5-a]pyridin-2-yl)-8-(methylamino)-2,7-naphthyridin-3-yl)cyclobutanecarboxamide

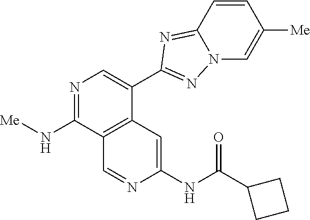 [SMILES: CNc1ncc(-c2nc3ccc(C)cn3n2)c2cc(NC(=O)C3CCC3)ncc12]

### Step 1: N¹-methyl-4-(6-methyl-[1,2,4]triazolo[1,5-a]pyridin-2-yl)-2,7-naphthyridine-1,6-diamine

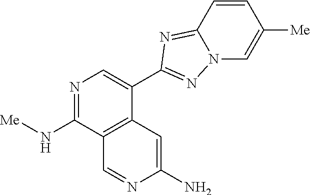 [SMILES: CNc1ncc(-c2nc3ccc(C)cn3n2)c2cc(N)ncc12]

To a stirred solution of N-(5-(6-methyl-[1,2,4]triazolo[1,5-a]pyridin-2-yl)-8-(methylamino)-2,7-naphthyridin-3-yl)cyclopropanecarboxamide (Example 503) (500.0 mg; 1.34 mmol; 1.00 eq.) MeOH/DMSO (3:5, 8 mL) was added a solution of NaOH (535.5 mg; 13.39 mmol; 10.00 eq.) in water (1 mL) at 0° C. The mixture was stirred at 60° C. overnight. The desired product was detected via LCMS. The reaction mixture was allowed to cool down to room temperature and concentrated under reduced pressure. The residue was purified by flash chromatography on pre-packed C18 column using 20-60% of MeCN in water (10 mmol/L NH₄HCO₃) to provide N¹-methyl-4-(6-methyl-[1,2,4]triazolo[1,5-a]pyridin-2-yl)-2,7-naphthyridine-1,6-diamine as a light yellow solid (400.0 mg, 97.8%). LCMS (ESI) m/z 306.1, [M+H]⁺.

### Step 2: N-(5-(6-methyl-[1,2,4]triazolo[1,5-a]pyridin-2-yl)-8-(methylamino)-2,7-naphthyridin-3-yl)cyclobutanecarboxamide

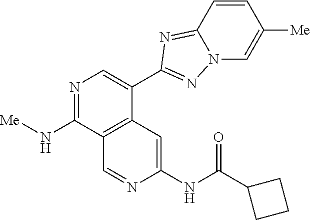 [SMILES: CNc1ncc(-c2nc3ccc(C)cn3n2)c2cc(NC(=O)C3CCC3)ncc12]

To a stirred solution of N¹-methyl-4-(6-methyl-[1,2,4]triazolo[1,5-a]pyridin-2-yl)-2,7-naphthyridine-1,6-diamine (70.0 mg; 0.229 mmol; 1.00 eq.) and cyclobutanecarboxylic acid (18.4 mg; 0.18 mmol; 0.80 eq.) in pyridine (2 mL) was added POCl₃ (105.4 mg; 0.69 mmol; 3.00 eq.) dropwise at 0° C. The resulting mixture was stirred at room temperature for 0.5 hour under nitrogen atmosphere. The desired product was detected via LCMS. The resulting mixture was quenched with water (10 mL) and extracted with EtOAc (3×20 mL). The combined organic layers were washed with brine (2×5 mL), dried over anhydrous Na₂SO₄, filtered, and concentrated under reduced pressure. The residue was purified by flash chromatography on silica gel column using 5-8% of MeOH in CH₂Cl₂ as eluent to provide the crude product. The crude product was repurified by recrystallization with a mixture solvent of MeOH/CH₂Cl₂ (1:10, 5 mL) to afford N-(5-(6-methyl-[1,2,4]triazolo[1,5-a]pyridin-2-yl)-8-(methylamino)-2,7-naphthyridin-3-yl)cyclobutanecarboxamide as a white solid (28.5 mg, 31.7%). LCMS (ESI) m/z 388.2, [M+H]⁺. ¹H NMR (400 MHz, DMSO-d₆) δ 10.45 (s, 1H), 9.56 (s, 1H), 9.39 (s, 1H), 8.83 (d, J=2.0 Hz, 1H), 8.80 (s, 1H), 8.31-8.23 (m, 1H), 7.78 (d, J=9.6 Hz, 1H), 7.57 (dd, J=9.6, 2.0 Hz, 1H), 3.50-3.37 (m, 1H), 3.05 (d, J=4.4 Hz, 3H), 2.43 (s, 3H), 2.34-2.19 (m, 2H), 2.19-2.06 (m, 2H), 2.03-1.90 (m, 1H), 1.88-1.76 (m, 1H).

### Example 562: Synthesis of (1S,2R)-2-methyl-N-(5-(6-methyl-[1,2,4]triazolo[1,5-a]pyridin-2-yl)-8-(methylamino)-2,7-naphthyridin-3-yl)cyclopropane-1-carboxamide

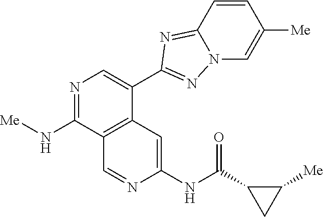 [SMILES: CNc1ncc(-c2nc3ccc(C)cn3n2)c2cc(NC(=O)[C@H]3C[C@H]3C)ncc12]

(1S,2R)-2-methyl-N-(5-(6-methyl-[1,2,4]triazolo[1,5-a]pyridin-2-yl)-8-(methylamino)-2,7-naphthyridin-3-yl)cyclopropane-1-carboxamide was synthesized using a similar procedure that was previously described in Example 561, by using N¹-methyl-4-(6-methyl-[1,2,4]triazolo[1,5-a]pyridin-2-yl)-2,7-naphthyridine-1,6-diamine (Example 561, Step 1) and (1S,2R)-2-methylcyclopropane-1-carboxylic acid as the starting material. LCMS (ESI) m/z 388.2, [M+H]⁺. ¹H NMR (400 MHz, DMSO-d₆) δ 10.81 (s, 1H), 9.48 (s, 1H), 9.40 (s, 1H), 8.82-8.76 (m, 2H), 8.31-8.22 (m, 1H), 7.76 (d, J=9.6 Hz, 1H), 7.55 (dd, J=9.6, 2.0 Hz, 1H), 3.06 (d, J=4.4 Hz, 3H), 2.42 (s, 3H), 2.14-2.04 (m, 1H), 1.34-1.22 (m, 1H), 1.14 (d, J=6.0 Hz, 3H), 1.04-0.94 (m, 1H), 0.89-0.80 (m, 1H).

### Example 563: Synthesis of N-(5-(6-((1R,4R)-2-oxa-5-azabicyclo[2.2.1]heptan-5-yl)-[1,2,4]triazolo[1,5-a]pyridin-2-yl)-8-(methylamino)-2,7-naphthyridin-3-yl)-1-fluorocyclopropane-1-carboxamide

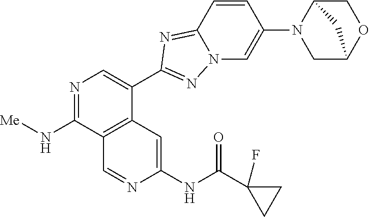 [SMILES: CNc1ncc(-c2nc3ccc(N4C[C@H]5C[C@@H]4CO5)cn3n2)c2cc(NC(=O)C3(F)CC3)ncc12]

### Step 1: 4-(6-((1R,4R)-2-oxa-5-azabicyclo[2.2.1]heptan-5-yl)-[1,2,4]triazolo[1,5-a]pyridin-2-yl)-N¹-methyl-2,7-naphthyridine-1,6-diamine

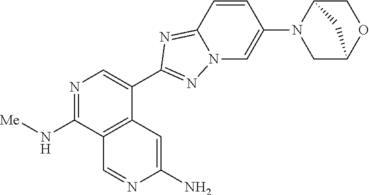 [SMILES: CNc1ncc(-c2nc3ccc(N4C[C@H]5C[C@@H]4CO5)cn3n2)c2cc(N)ncc12]

To a stirred solution of N-(5-(6-((1R,4R)-2-oxa-5-azabicyclo[2.2.1]heptan-5-yl)-[1,2,4]triazolo[1,5-a]pyridin-2-yl)-8-(methylamino)-2,7-naphthyridin-3-yl)cyclopropanecarboxamide (Example 489) (1.500 mg; 0.329 mmol; 1.00 eq.) in MeOH/DMSO (3:5, 6 mL) was added a solution of NaOH (131.4 mg; 3.290 mmol; 10.00 eq.) in water (2 mL) at 0° C. The mixture was stirred at 60° C. overnight. The desired product was detected via LCMS. The mixture was allowed to cool down to room temperature. The resulting mixture was concentrated under reduced pressure. The residue was purified by flash chromatography on pre-packed C18 column using 20-60% of MeCN in water (10 mmol/L NH₄HCO₃) as eluent to provide 4-(6-((1R,4R)-2-oxa-5-azabicyclo[2.2.1]heptan-5-yl)-[1,2,4]triazolo[1,5-a]pyridin-2-yl)-N¹-methyl-2,7-naphthyridine-1,6-diamine as a green solid (90.0 mg, 70.5%). LCMS (ESI) m/z 389.2, [M+H]⁺.

### Step 2: N-(5-(6-((1R,4R)-2-oxa-5-azabicyclo[2.2.1]heptan-5-yl)-[1,2,4]triazolo[1,5-a]pyridin-2-yl)-8-(methylamino)-2,7-naphthyridin-3-yl)-1-fluorocyclopropane-1-carboxamide

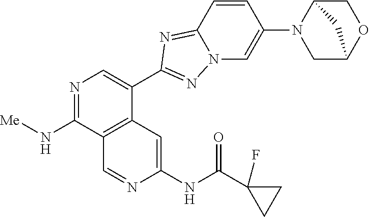 [SMILES: CNc1ncc(-c2nc3ccc(N4C[C@H]5C[C@@H]4CO5)cn3n2)c2cc(NC(=O)C3(F)CC3)ncc12]

To a stirred solution of 4-(6-((1R,4R)-2-oxa-5-azabicyclo[2.2.1]heptan-5-yl)-[1,2,4]triazolo[1,5-a]pyridin-2-yl)-N¹-methyl-2,7-naphthyridine-1,6-diamine (80.0 mg; 0.21 mmol; 1.00 eq.) and 1-fluorocyclopropane-1-carboxylic acid (16.1 mg; 0.154 mmol; 0.75 eq.) in pyridine (2.5 mL) was added POCl₃ (94.7 mg; 0.62 mmol; 3.00 eq.) dropwise at 0° C. The resulting mixture was stirred at room temperature for 0.5 hour under nitrogen atmosphere. The desired product was detected via LCMS. The resulting mixture was diluted with water (10 mL) and extracted with EtOAc (3×20 mL). The combined organic layers were washed with brine (3×5 mL) and dried over anhydrous Na₂SO₄, filtered, and concentrated under reduced pressure. The residue was purified by flash chromatography on silica gel column using 5-8% of MeOH in CH₂Cl₂ as eluent to provide the crude product. The crude product was purified by recrystallization with MeOH/CH₂Cl₂ (1:10, 8 mL) to afford N-(5-(6-((1R,4R)-2-oxa-5-azabicyclo[2.2.1]heptan-5-yl)-[1,2,4]triazolo[1, 5-a]pyridin-2-yl)-8-(methyl amino)-2,7-naphthyridin-3-yl)-1-fluorocyclopropane-1-carboxamide as a light yellow solid (45.2 mg, 45.5%). LCMS (ESI) m/z 475.2, [M+H]⁺. ¹H NMR (400 MHz, DMSO-d₆) δ 10.32 (s, 1H), 9.46 (s, 1H), 9.44 (s, 1H), 8.78 (s, 1H), 8.34-8.27 (m, 1H), 8.26 (d, J=2.0 Hz, 1H), 7.73 (d, J=9.6 Hz, 1H), 7.41 (dd, J=9.6, 2.0 Hz, 1H), 4.71-4.65 (m, 2H), 3.80-3.74 (m, 2H), 3.62-3.56 (m, 1H), 3.07 (d, J=4.4 Hz, 3H), 3.06-3.01 (m, 1H), 2.02-1.95 (m, 1H), 1.92-1.85 (m, 1H), 1.54-1.34 (m, 4H).

### Example 564 and 565: Synthesis of N-(5-([1,2,4]triazolo[1,5-a]pyridin-2-yl)-8-((methyl-d₃)amino)-2,7-naphthyridin-3-yl)-2-fluorocyclopropane-1-carboxamide (tran racemate) (Example 564) and N-(5-([1,2,4]triazolo[1,5-a]pyridin-2-yl)-8-((methyl-d₃)amino)-2,7-naphthyridin-3-yl)-2-fluorocyclopropane-1-carboxamide (cis racemate) (Example 565)

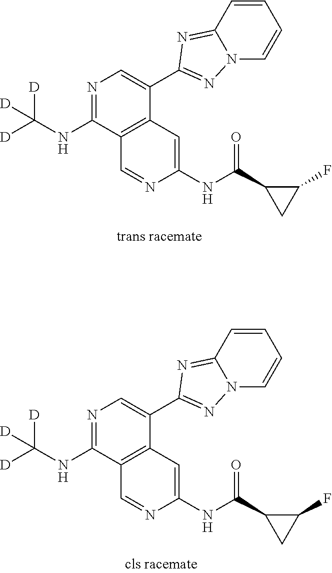 [SMILES: [2H]C([2H])([2H])Nc1ncc(-c2nc3ccccn3n2)c2cc(NC(=O)[C@@H]3C[C@H]3F)ncc12 ; [2H]C([2H])([2H])Nc1ncc(-c2nc3ccccn3n2)c2cc(NC(=O)[C@@H]3C[C@@H]3F)ncc12]

### Step 1: 2-fluoro-N-(8-((methyl-d₃)amino)-2,7-naphthyridin-3-yl)cyclopropane-1-carboxamide

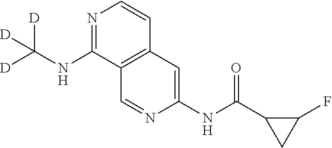 [SMILES: [2H]C([2H])([2H])Nc1nccc2cc(NC(=O)C3CC3F)ncc12]

To a stirred mixture of 6-chloro-N-(methyl-d₃)-2,7-naphthyridin-1-amine (220.0 mg; 1.122 mmol; 1.00 eq.) in 1,4-dioxane (7 mL) was added Pd₂(dba)₃ (102.6 mg; 0.1.1 mmol; 0.10 eq.), XantPhos (129.7 mg; 0.22 mmol; 0.20 eq.), Cs₂CO₃ (731.5 mg; 2.24 mmol; 2.00 eq.) and 2-fluorocyclopropane-1-carboxamide (trans racemate) (231.1 mg; 2.24 mmol; 2.00 eq.). The mixture was stirred at 90° C. for 3 hours under nitrogen atmosphere. The desired product was detected via LCMS. The resulting mixture was concentrated under vacuum. The residue was purified by flash chromatography on silica gel column using 2-10% of MeOH in CH₂Cl₂ as eluent to provide 2-fluoro-.N-(8-((methyl-d₃)amino)-2,7-naphthyridin-3-yl)cyclopropane-1-1.0 carboxamide as a black solid (220.0 mg, 74.5%). LCMS (ESI) m/z 264.1, [M+H]⁺.

### Step 2: N-(5-bromo-8-((methyl-d₃)amino)-2,7-naphthyridin-3-yl)-2-fluorocyclopropane-1-carboxamide

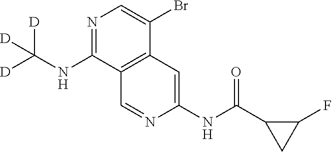 [SMILES: [2H]C([2H])([2H])Nc1ncc(Br)c2cc(NC(=O)C3CC3F)ncc12]

To a stirred solution of 2-fluoro-N-(8-((methyl-d₃)amino)-2,7-naphthyridin-3-yl)cyclopropane-1-carboxamide (220.0 mg; 0.836 mmol; 1.00 eq.) in DMF (5 mL) was added NIBS (149.0 mg; 0.837 mmol; 1.00 eq.) at 0° C. The reaction was stirred at room temperature for 1 hour under nitrogen atmosphere. The desired product was detected via LCMS. The mixture was diluted with EtOAc (90 mL) and washed with brine (3×5 mL). The organic layer was dried with Na₂SO₄ and concentrated under reduced pressure. The residue was purified by flash chromatography on silica gel column using 2-10% of MeOH in CH₂Cl₂ as eluent to provide N-(5-bromo-8-((methyl-d₃)amino)-2,7-naphthyridin-3-yl)-2-fluorocyclopropane-1-carboxamide as a yellow solid (135.0 mg, 47.0%). LCMS (ESI) m/z 342.0, [M+H]⁺.

### Step 3: 2-fluoro-N-(8-((methyl-d₃)amino)-5-(4,4,5,5-tetramethyl-1,3,2-dioxaborolan-2-yl)-2,7-naphthyridin-3-yl)cyclopropane-11-carboxamide (mixture of trans and cis)

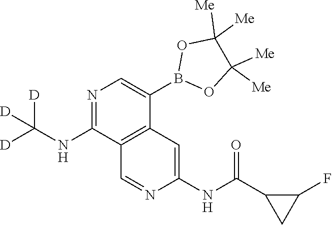 [SMILES: [2H]C([2H])([2H])Nc1ncc(B2OC(C)(C)C(C)(C)O2)c2cc(NC(=O)C3CC3F)ncc12]

To a solution of N-(5-bromo-8-((methyl-d₃)amino)-2,7-naphthyridin-3-yl)-2-fluorocyclopropane-1-carboxamide (135.0 mg; 0.395 mmol; 1.00 eq.) in 1,4-dioxane (4.5 mL) was added 4,4,5,5-tetramethyl-2-(4,4,5,5-tetramethyl-1,3,2-dioxaborolan-2-yl)-1,3,2-dioxaborolane (1.00 g; 3.950 mmol; 10.00 eq.), Pd(dppf)Cl₂ (28.9 mg; 0.039 mmol; 0.10 eq.) and KOAc (77.4 mg; 0.790 mmol; 2.00 eq.). The reaction was stirred at 100° C. for 2 hours under nitrogen atmosphere. The desired product was detected via LCMS. The resulting mixture was concentrated under vacuum. The residue was slurried in petroleum ether/EtOAc (10:1, 7 mL) at room temperature for 3 hours whereupon the precipitated product were isolated via filtration and washed with petroleum ether/EtOAc(10:1, 3 mL). The precipitated product were dried under reduced pressure to afford 2-fluoro-N-(8-((methyl-d₃)amino)-5-(4,4,5,5-tetramethyl-1,3,2-dioxaborolan-2-yl)-2,7-naphthyridin-3-yl)cyclopropane-1-carboxamide as a black solid (190.0 mg, crude). LCMS (ESI) m/z 390.2, [M+H]⁺.

### Step 4: N-(5-([1,2,4]triazolo[1,5-a]pyridin-2-yl)-8-((methyl-d₃)amino)-2,7-naphthyridin-3-yl)-2-fluorocyclopropane-1-carboxamide (trans racemate) and N-(5-([1,2,4]triazolo[1,5-a]pyridin-2-yl)-8-((methyl-d₃)amino)-2,7-naphthyridin-3-yl)-2-fluorocyclopropane-1-carboxamide (cis racemate)

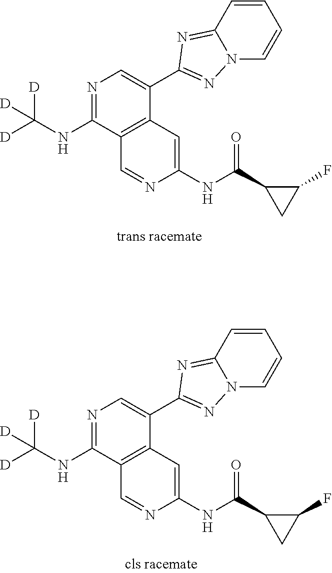 [SMILES: [2H]C([2H])([2H])Nc1ncc(-c2nc3ccccn3n2)c2cc(NC(=O)[C@@H]3C[C@H]3F)ncc12 ; [2H]C([2H])([2H])Nc1ncc(-c2nc3ccccn3n2)c2cc(NC(=O)[C@@H]3C[C@@H]3F)ncc12]

To a stirred mixture of 2-fluoro-N-(8-((methyl-d₃)amino)-5-(4,4,5,5-tetramethyl-1,3,2-dioxaborolan-2-yl)-2,7-naphthyridin-3-yl)cyclopropane-1-carboxamide (180.0 mg; 0.46 mmol; 1.00 eq.) in a mixture solvent of 1,4-dioxane/water (5:1, 6 mL) were added XPhos Pd G₃ (39.1 mg; 0.05 mmol; 0.10 eq.), XPhos (22.0 mg; 0.05 mmol; 0.10 eq.), K₃PO₄ (293.5 mg; 1.38 mmol; 2.99 eq.) and 2-chloro-[1,2,4]triazolo[1,5-a]pyridine (106.2 mg; 0.69 mmol, 1.50 eq.). The reaction was stirred at 90° C. for 1 hour under nitrogen atmosphere. The desired product was detected via LCMS. The resulting mixture was concentrated under vacuum. The residue was purified by flash chromatography, on silica gel column, using 2-10% of MeOH in CH₂Cl₂ as eluent to provide the crude product. The crude product was purified by flash chromatography on pre-packed C18 column using 20-70% of MeCN in water (10 mmol/L NH₄HCO₃) as eluent to provide N-(5-([1,2,4]triazolo[1,5-a]pyridin-2-yl)-8-((methyl-d₃)amino)-2,7-naphthyridin-3-yl)-2-fluorocyclopropane-1-carboxamide as a white solid (Example 564, trans racemate) (4.1 mg, 2.3%) and N-(5-([1,2,4]triazolo[1,5-a]pyridin-2-yl)-8-((methyl-d₃)amino)-2,7-naphthyridin-3-yl)-2-fluorocyclopropane-3-carboxamide as a white solid (Example 564, cis racemate) (14.1 mg, 8.0%). LCMS (ESI) m/z 381.2, [M+H]⁺.

HNMR for Example 564: ¹H NMR (400 MHz, Methanol-d₄) δ 9.55 (s, 1H), 9.26 (s, 1H), 8.90 (d, J=6.8 Hz, 1H), 8.83 (s, 1H), 7.79 (d, J=8.8 Hz, 1H), 7.76-7.67 (m, 1H), 7.25-7.16 (m, 1H), 5.02-4.95 (m, 1H), 2.58-2.41 (m, 1H), 1.60-1.38 (m, 2H).

HNMR for Example 565: ¹H NMR (400 MHz, DMSO-d₆) δ 10.98 (s, 1H), 9.52 (s, 1H), 9.42 (s, 1H), 8.96 (d, J=6.8 Hz, 1H), 8.82 (s, 1H), 8.33-8.25 (m, 1H), 7.87 (d, J=8.8 Hz, 1H), 7.74-7.65 (m, 1H), 7.24-7.18 (m, 1H), 5.07-4.84 (m, 1H), 2.34-2.22 (m, 1H), 1.76-1.62 (m, 3H), 1.26-1.13 (m, 1H).

### Example 566: Synthesis of (1R,2R)—N-(5-([1,2,4]triazolo[1,5-a]pyridin-2-yl)-8-((methyl-d₃)amino)-2,7-naphthyridin-3-yl)-2-fluorocyclopropane-1-carboxamide

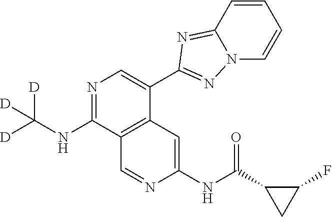 [SMILES: [2H]C([2H])([2H])Nc1ncc(-c2nc3ccccn3n2)c2cc(NC(=O)[C@H]3C[C@H]3F)ncc12]

### Step 1: N-(5-([1,2,4]triazolo[1,5-a]pyridin-2-yl)-8-(methyl-d₃)amino)-2,7-naplathyridin-3-yl)cyclopropanecarboxamide

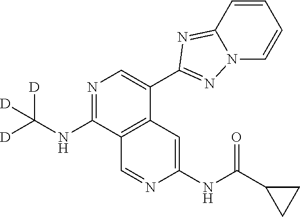 [SMILES: [2H]C([2H])([2H])Nc1ncc(-c2nc3ccccn3n2)c2cc(NC(=O)C3CC3)ncc12]

A suspension of N-(8-((methyl-d₃)amino)-5-(4,4,5,5-tetramethyl-1,3,2-dioxaborolan-2-yl)-2,7-naphthyridin-3-yl)cyclopropanecarboxamide (483.5 mg; 1.302 mmol; 1.00 eq.), 2-chloro-[1,2,4]triazolo[1,5-a]pyridine (200.0 mg; 1.302 mmol; 1.00 eq.), XPhos Pd G₃ (330.7 mg; 0.391 mmol; 0.30 eq.), XPhos (186.3 mg; 0.391 mmol; 0.30 eq.) and K₃PO₄ (829.3 mg; 3.907 mmol; 3.00 eq.) in 1,4-dioxane/water (5:1, 6 mL) was stirred at 110° C. for 1 hour under nitrogen atmosphere. Upon completion, the resulting mixture was concentrated under reduced pressure. The residue was purified by flash chromatography, on silica gel column, using 5-10% of MeOH: in CH₂Cl₂ as eluent to afford N-(5-([1,2,4]triazolo[1,5-a]pyridin-2-yl)-8-((methyl-d₃)amino)-2,7-naphthyridin-3-yl)cyclopropanecarboxamide as a yellow solid (250.0 mg, 50.3%). LCMS (ESI) m/z 363.2, [M+H]⁺.

### Step 2: 4-([1,2,4]triazolo[1,5-a]pyridin-2-yl)-N¹-(methyl-d₃)-2,7-naphthyridine-1,6-diamine

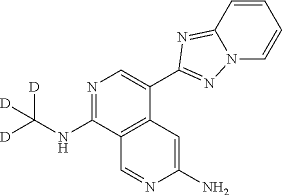 [SMILES: [2H]C([2H])([2H])Nc1ncc(-c2nc3ccccn3n2)c2cc(N)ncc12]

To a solution of N-(5-([1,2,4]triazolo[1,5-a]pyridin-2-yl)-8-((methyl-d₃)amino)-2,7-naphthyridin-3-yl)cyclopropanecarboxamide (200.0 mg; 0.552 mmol; 1.00 eq.) in MeOH/DMSO (5:1, 18 mL) was added a solution of NaOH (220.8 mg; 5.520 mmol; 10.00 eq.) in water (4.5 mL) at 0° C. The resulting mixture was stirred at 60° C. overnight. The reaction mixture was cooled, concentrated under vacuum. The resulting mixture was added dropwise to water (100 mL), whereupon the precipitated product was collected by filtration and washed with water (5 mL) to afford 4-([1,2,4]triazolo[1,5-a]pyridin-2-yl)-N¹-(methyl-d₃)-2,7-naphthyridine-1,6-diamine as a brown solid (160.0 mg, 94.6%). LCMS (ESI) m/z 295.1, [M+H]⁺.

### Step 3: (1R,2R)—N-(5-([1,2,4]triazolo[1,5-a]pyridin-2-yl)-8-((methyl-d₃)amino)-2,7-naphthyridin-3-371)-2-fluorocyclopropane-1-carboxamide

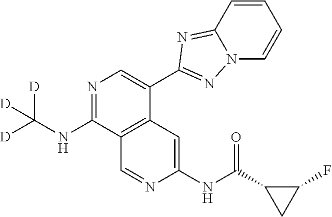 [SMILES: [2H]C([2H])([2H])Nc1ncc(-c2nc3ccccn3n2)c2cc(NC(=O)[C@H]3C[C@H]3F)ncc12]

To a mixture of 4-([1,2,4]triazolo[1,5-a]pyridin-2-yl)-N¹-(methyl-d₃)-2,7-naphthyridine-1,6-diamine (80.0 mg; 0.272 mmol; 1.00 eq.) and (1R,2R)-2-fluorocyclopropane-1-carboxylic acid (25.5 mg; 0.245 mmol; 0.90 eq.) in pyridine (5 mL) was added POCl₃ (125.0 mg; 0.815 mmol; 3.00 eq.) dropwise at 0° C. The resulting mixture was stirred at room temperature for 0.5 hour under nitrogen atmosphere. LC-MS indicated that the starting material was consumed. The mixture was diluted with EtOAc (100 mL), washed with brine (2×5 mL). The organic layer was dried over Na₂SO₄ and concentrated under reduced pressure. The residue was purified by flash chromatography on silica gel column using 5-10% of MeOH in CH₂Cl₂ as eluent to afford the crude product. The crude product was purified by flash chromatography on pre-packed C18 column using 30-60% of MeOH in water (10 mmol/L NH₄HCO₃) as eluent to provide (1R,2R)—N-(5-([1,2,4]triazolo[1,5-a]pyridin-2-yl)-8-((methyl-d₃)amino)-2,7-naphthyridin-3-yl)-2-fluorocyclopropane-1-carboxamide as a yellow solid (21.7 mg, 20.6%). LCMS (ESI) m/z 381.2, [M+H]⁺. ¹H NMR (400 MHz, DMSO-d₆) δ 11.03 (s, 1H), 9.53 (s, 1H), 9.45 (s, 1H), 9.00-8.91 (m, 1H), 8.79 (s, 1H), 8.60-8.30 (m, 1H), 7.91-7.83 (m, 1H), 7.75-7.65 (m, 1H), 7.25-7.14 (m, 1H), 5.10-4.80 (m, 1H), 2.32-2.20 (m, 1H), 1.76-1.62 (m, 1H), 1.25-1.15 (m, 1H).

### Example 567: Synthesis of (1S,2S)—N-(5-([1,2,4]triazolo[1,5-a]pyridin-2-yl)-8-((methyl-d₃)amino)-2,7-naphthyridin-3-yl)-2-fluorocyclopropane-1-carboxamide

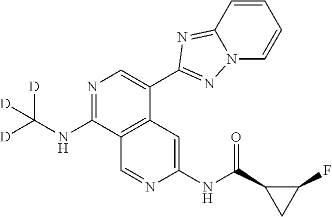 [SMILES: [2H]C([2H])([2H])Nc1ncc(-c2nc3ccccn3n2)c2cc(NC(=O)[C@@H]3C[C@@H]3F)ncc12]

(1S,2S)—N-(5-([1,2,4]triazolo[1,5-a]pyridin-2-yl)-8-((methyl-d₃)amino)-2,7-naphthyridin-3-yl)-2-fluorocyclopropane-1-carboxamide was synthesized using a similar procedure that was previously described in Example 566 by using 4-([1,2,4]triazolo[1,5-a]pyridin-2-yl)-N¹-(methyl-d₃)-2,7-naphthyridine-1,6-diamine and (1S,2S)-2-fluorocyclopropane-1-carboxylic acid as the starting material. LCMS (ESI) m/z 381.2, [M+H]⁺. ¹H NMR (400 MHz, DMSO-d₆) δ 10.99 (s, 1H), 9.52 (s, 1H), 9.43 (s, 1H), 8.99-8.93 (m, 1H), 8.81 (s, 1H), 8.40-8.25 (m, 1H), 7.91-7.84 (m, 1H), 7.74-7.65 (m, 1H), 7.24-7.17 (m, 1H), 5.06-4.85 (m, 1H), 2.36-2.22 (m, 1H), 1.76-1.62 (m, 1H), 1.26-1.13 (m, 1H).

### Example 568: Synthesis of (1R,2R)-2-fluoro-N-(5-(6-methoxy-[1,2,4]triazolo[1,5-a]pyridin-2-yl)-8-((methyl-d₃)amino)-2,7-naphthyridin-3-yl)cyclopropane-1-carboxamide

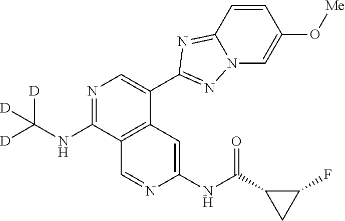 [SMILES: [2H]C([2H])([2H])Nc1ncc(-c2nc3ccc(OC)cn3n2)c2cc(NC(=O)[C@H]3C[C@H]3F)ncc12]

### Step 1: 4-(6-methoxy-[1,2,4]triazolo[1,5-a]pyridin-2-yl)-N¹-(methyl-d₃)-2,7-naphthyridine-1,6-diamine

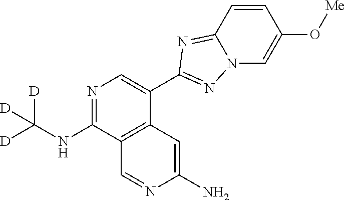 [SMILES: [2H]C([2H])([2H])Nc1ncc(-c2nc3ccc(OC)cn3n2)c2cc(N)ncc12]

To a stirred solution of N-(5-(6-methoxy-[1,2,4]triazolo[1,5-a]pyridin-2-yl)-8-((methyl-d₃)amino)-2,7-naphthyridin-3-yl)cyclopropanecarboxamide (Example 511) (280.0 mg; 0.713 mmol; 1.00 eq.) in DMSO/MeOH (1:5, 15.6 mL) was added a solution of NaOH (285.2 mg; 7.130 mmol; 10.00 eq.) in water (4 mL) at 0° C. The resulting mixture was stirred at 60° C. overnight. After the reaction was complete, MeOH was removed under vacuum. To the mixture was added water (50 mL), whereupon the precipitated product were collected by filtration and washed with water (2×10 mL) to afford 4-(6-methoxy-[1,2,4]triazolo[1,5-a]pyridin-2-yl) N¹-(methyl-d₃)-2,7-naphthyridine-1,6-diamine as a yellow solid (160.0 mg, 68.3%). LCMS (ESI) m/z 325.1, [M+H]⁺.

### Step 2: (1R,2R)-2-fluoro-N-(5-(6-methoxy-[1,2,4]triazolo[1,5-a]pyridin-2-yl)-8-((methyl-d₃)amino)-2,7-naphthyridin-3-yl)cyclopropane-1-carboxamide

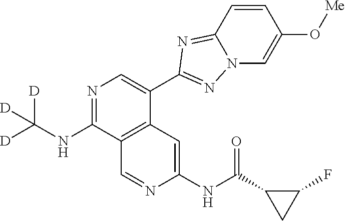 [SMILES: [2H]C([2H])([2H])Nc1ncc(-c2nc3ccc(OC)cn3n2)c2cc(NC(=O)[C@H]3C[C@H]3F)ncc12]

To a solution of 4-(6-methoxy-[1,2,4]triazolo[1,5-a]pyridin-2-yl)-N¹-(methyl-d₃)-2,7-naphthyridine-1,6-diamine (70.0 mg; 0.22 mmol; 1.00 eq.) and (1R,2R)-2-fluorocyclopropane-1-carboxylic acid (20.2 mg; 0.19 mmol; 0.90 eq.) in pyridine (2.5 mL) was added POCl₃ (99.3 mg; 0.65 mmol; 3.00 eq.) at 0° C. under nitrogen atmosphere. The resulting mixture was stirred at room temperature for 0.5 hour. LC-MS indicated that the reaction was complete. After the mixture was quenched by water (2 mL), the solution was purified by flash chromatography on pre-packed C18 column using 20-100% of MeOH in water (10 mmol/L NH₄HCO₃) as eluent to provide (1R,2R)-2-fluoro-N-(5-(6-methoxy-[1,2,4]triazolo[1,5-a]pyridin-2-yl)-8-((methyl-d₃)amino)-2,7-naphthyridin-3-yl)cyclopropane-1-carboxamide as a yellow solid (59.6 mg, 63.4%). LCMS (ESI) m/z 411.2, [M+H]⁺. ¹H NMR (400 MHz, DMSO-d₆) δ 11.45 (s, 1H), 9.64 (s, 1H), 9.54 (s, 1H), 8.73 (s, 1H), 8.72 (d, J=2.0 Hz, 1H), 8.45-8.35 (m, 1H), 7.86 (d, J=9.6 Hz, 1H), 7.53 (dd, J=9.6, 2.0 Hz, 1H), 5.10-4.81 (m, 1H), 3.92 (s, 3H), 2.34-2.28 (m, 1H), 1.77-1.65 (m, 1H), 1.29-1.19 (m, 1H).

### Example 569: Synthesis of (1S,2S)-2-fluoro-N-(5-(6-methoxy-[1,2,4]triazolo[1,5-a]pyridin-2-yl)-8-((methyl-d₃)amino)-2,7-naphthyridin-3-yl)cyclopropane-1-carboxamide

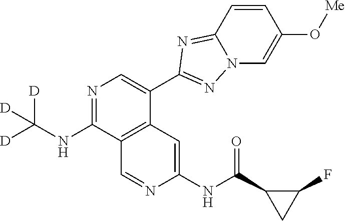 [SMILES: [2H]C([2H])([2H])Nc1ncc(-c2nc3ccc(OC)cn3n2)c2cc(NC(=O)[C@@H]3C[C@@H]3F)ncc12]

(1S,2S)-2-fluoro-N-(5-(6-methoxy-[1,2,4]triazolo[1,5-a]pyridin-2-yl)-8-((methyl-d₃)amino)-2,7-naphthyridin-3-yl)cyclopropane-1-carboxamide was synthesized using a similar procedure that was previously described in Example 568 by using 4-(6-methoxy-[1,2,4]triazolo[1,5-a]pyridin-2-yl)-N^{a}-(methyl-d₃)-2,7-naphthyridine-1,6-diamine and (1S,2S)-2-fluorocyclopropane-1-carboxylic acid as the starting material. LCMS (ESI) m/z 411.2, [M+H]⁺. ¹H NMR (400 MHz, DMSO-d₆) δ 1.0.98 (s, 1H), 9.42 (s, 1H), 9.41 (s, 1H), 8.74 (s, 1H), 8.69 (d, J=2.0 Hz, 1H), 8.29-8.21 (s, 1H), 7.80 (d, J=9.6 Hz, 1H), 7.46 (dd, J=9.6, 2.0 Hz, 1H), 5.07-4.84 (m, 1H), 3.91 (s, 3H), 2.31-2.22 (m, 1H), 1.78-1.62 (m, 1H), 1.21-1.13 (m, 1H).

### Example 570: Synthesis of(1S,2R)—N-(5-(6-methoxy-[1,2,4]triazolo[1,5-a]pyridin-2-yl)-8-((methyl-d₃)amino)-2,7-naphthyridin-3-yl)-2-methylcyclopropane-1-carboxamide

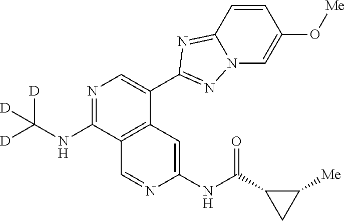 [SMILES: [2H]C([2H])([2H])Nc1ncc(-c2nc3ccc(OC)cn3n2)c2cc(NC(=O)[C@H]3C[C@H]3C)ncc12]

(1S,2R)—N-(5-(6-methoxy-[1,2,4]triazolo[1,5-a]pyridin-2-yl)-8-((methyl-d₃)amino)-2,7-naphthyridin-3-yl)-2-methylcyclopropane-1-carboxamide was synthesized using a similar procedure that was previously described in Example 568, by using 4-(6-methoxy-[1,2,4]triazolo[1,5-a]pyridin-2-yl)-N¹-(methyl-d₃)-2,7-naphthyridine-1,6-diamine and (1S,2R)-2-methylcyclopropane-1-carboxylic acid as the starting material. LCMS (ESI) m/z 407.2, [M+H]⁺. ¹H NMR (400 MHz, DMSO-d₆) δ 10.92 (s, 1H), 9.47 (s, 1H), 9.43 (s, 1H), 8.68 (s, 1H), 8.67 (d, J=2.0 Hz, 1H), 8.53-8.39 (m, 1H), 7.80 (d, J=9.6 Hz, 1H), 7.49 (d, J=9.6, 2.0 Hz, 1H), 3.91 (s, 3H), 2.15-2.04 (m, 1H), 1.38-1.27 (m, 1H), 1.12 (d, J=6.0 Hz, 3H), 1.06-0.96 (m, 1H), 0.91-0.80 (m, 1H).

### Example 571: Synthesis of 1-fluoro-N-(8-((methyl-d₃)amino)-5-(6-morpholino-[1,2,4]triazolo[1,5-a]pyridin-2-yl)-2,7-naphthyridin-3-yl)cyclopropane-1-carboxamide

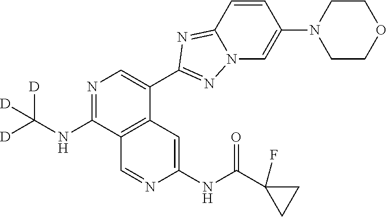 [SMILES: [2H]C([2H])([2H])Nc1ncc(-c2nc3ccc(N4CCOCC4)cn3n2)c2cc(NC(=O)C3(F)CC3)ncc12]

### Step 1: N-(8-((methyl-d₃)amino)-5-(6-morpholino-[1,2,4]triazolo[1,5-a]pyridin-2-yl)-2,7-naphthyridin-3-yl)cyclopropanecarboxamide

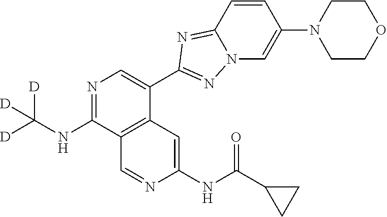 [SMILES: [2H]C([2H])([2H])Nc1ncc(-c2nc3ccc(N4CCOCC4)cn3n2)c2cc(NC(=O)C3CC3)ncc12]

A stirring mixture of N-(8-((methyl-d₃)amino)-5-(4,4,5,5-tetramethyl-1,3,2-dioxaborolan-2-yl)-2,7-naphthyridin-3-yl)cyclopropanecarboxamide (466.6 mg; 1.257 mmol; 1.00 eq.), 4-(2-chloro-[1,2,4]triazolo[1,5-a]pyridin-6-yl)morpholine (300.0 mg; 1.257 mmol; 1.00 eq.), XPhos Pd G₃ (319.2 mg; 0.377 mmol; 0.30 eq.), XPhos (179.8 mg; 0.377 mmol; 0.30 eq.), K₃PO₄ (800.4 mg; 3.771 mmol; 3.00 eq.) in 1,4-dioxane/water (5:1, 12 mL) was reacted at 110° C. for 2 hours under nitrogen atmosphere. The desired product was detected via LCMS. The mixture was concentrated under reduced pressure. The residue was purified by flash chromatography on silica gel column using 0-1.5% of MeOH in CH₂Cl₂ as eluent to provide N-(8-((methyl-d₃)amino)-5-(6-morpholino-[1,2,4]triazolo[1,5-a]pyridin-2-yl)-2,7-naphthyridin-3-yl)cyclopropanecarboxamide as a brown solid (155.0 mg, 27.6%). LCMS (ESI) m/z 448.2, [M+H]⁺.

### Step 2: N¹-(methyl-d₃)-4-(6-morpholino-[1,2,4]triazolo[1,5-a]pyridin-2-yl)-2,7-naphthyridine-1,6-diamine

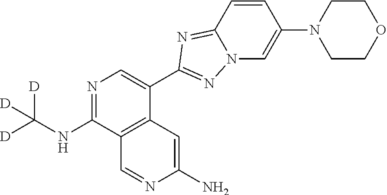 [SMILES: [2H]C([2H])([2H])Nc1ncc(-c2nc3ccc(N4CCOCC4)cn3n2)c2cc(N)ncc12]

To a solution of N-(8-((methyl-d₃)amino)-5-(6-morpholino-[1,2,4]triazolo[1,5-a]pyridin-2-yl)-2,7-naphthyridin-3-yl)cyclopropanecarboxamide (155.5 mg; 0.347 mmol; 1.00 eq.) in a mixture solution of DMSO/MeOH (1:5, 12 mL) was added a solution of NaOH (138.8 mg; 3.470 mmol; 10.00 eq.) in water (1.5 mL) at 0° C. The reaction was stirred at 60° C. overnight. The reaction mixture was cooled and MeOH was removed under vacuum. The remaining mixture was added dropwise to water (100 mL), whereupon the precipitated product was isolated via filtration and washed with water (3×10 mL) to afford N¹-(methyl-d₃)-4-(6-morpholino-[1,2,4]triazolo[1,5-a]pyridin-2-yl)-2,7-naphthyridine-1,6-diamine as a grey solid (100.0 mg, 75.9%). LCMS (ESI) m/z 380.2, [M+H]⁺.

### Step 3: 1-fluoro-N-(8-((methyl-d₃)amino)-5-(6-morpholino-[1,2,4]triazolo[1,5-a]pyridin-2-yl)-2,7-naphthyridin-3-yl)cyclopropane-1-carboxamide

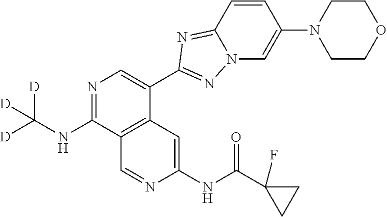 [SMILES: [2H]C([2H])([2H])Nc1ncc(-c2nc3ccc(N4CCOCC4)cn3n2)c2cc(NC(=O)C3(F)CC3)ncc12]

To a stirred solution of N¹-(methyl-d₃)-4-(6-morpholino-[1,2,4]triazolo[1,5-a]pyridin-2-yl)-2,7-naphthyridine-1,6-diamine (100.0 mg; 0.264 mmol; 1.00 eq.), 1-fluorocyclopropane-1-carboxylic acid (24.7 mg; 0.24 mmol; 0.90 eq.) in pyridine (5 mL) was added POCl₃ dropwise (121.2 mg; 0.79 mmol; 3.00 eq.) at 0° C. under nitrogen atmosphere and reacted at room temperature for 0.5 h. The mixture was diluted with EtOAc (100 mL), washed with brine (3×5 mL). The combined organic layers were concentrated under reduced pressure. The residue was purified by flash chromatography, on silica gel column, using 10-30% of MeOH in CH₂Cl₂ as eluent to provide the crude product. The crude product was repurified by flash chromatography on pre-packed C18 column using 20-1.00% of MeOH in water (10 mmol/L NH₄HCO₃) as eluent to provide 1-fluoro-N-(8-((methyl-d₃)amino)-5-(6-morpholino-[1,2,4]triazolo[1,5-a]pyridin-2-yl)-2,7-naphthyridin-3-yl)cyclopropane-1-carboxamide as an off-white solid (35.1 mg, 28.3%). LCMS (ESI) m/z 466.2, [M+H]⁺. ¹H NMR (400 MHz, DMSO-d₆) δ 10.33 (s, 1H), 9.46 (s, 1H), 9.45 (s, 1H), 8.79 (s, 1H), 8.44-8.30 (m, 2H), 7.75 (d, J=9.6 Hz, 1H), 7.69 (dd, J=9.6, 2.0 Hz, 1H), 3.84-3.76 (m, 4H), 3.22-3.11 (m, 4H), 1.52-1.37 (m, 4H).

### Example 572: Synthesis of (1S,2R)-2-methyl-N-(8-((methyl-d₃)amino)-5-(6-morpholino-[1,2,4]triazolo[1,5-a]pyridin-2-yl)-2,7-naphthyridin-3-yl)cyclopropane-1-carboxamide

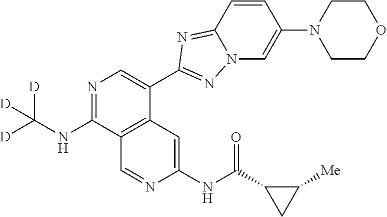 [SMILES: [2H]C([2H])([2H])Nc1ncc(-c2nc3ccc(N4CCOCC4)cn3n2)c2cc(NC(=O)[C@H]3C[C@H]3C)ncc12]

To a solution of N¹-(methyl-d₃)-4-(6-morpholino-[1,2,4]triazolo[1,5-a]pyridin-2-yl)-2,7-naphthyridine-1,6-diamine (Example 571, Step 2) (70.0 mg; 0.184 mmol; 1.00 eq.) and (1S,2R)-2-methylcyclopropane-1-carboxylic acid (1.6.6 mg; 0.166 mmol; 0.90 eq.) in pyridine (5 mL) was added POCl₃ (84.9 mg; 0.554 mmol; 3.00 eq.) dropwise at 0° C. under nitrogen atmosphere. The resulting mixture was stirred at room temperature for 0.5 hour. The desired product was detected via LCMS. The mixture was diluted with EtOAc (90 mL) and washed with brine (3×5 mL). The organic layer was dried over Na₂SO₄ and the solvent was removed under reduced pressure. The residue was purified by flash chromatography on silica gel column using 5-10% of MeOH in CH₂Cl₂ as eluent to afford the crude product. The crude product was purified by flash chromatography on pre-packed C18 column using 60-80% of MeOH in water (1.0 mmol/L NH₄HCO₃) as eluent to provide (1S,2R)-2-methyl-N-(8-((methyl-d₃)amino)-5-(6-morpholino-[1,2,4]triazolo[1,5-a]pyridin-2-yl)-2,7-naphthyridin-3-yl)cyclopropane-1-carboxamide as a yellow solid (23.0 mg, 26.6%). LCMS (ESI) m/z 462.2, [M+H]⁺. ¹H NMR (400 MHz, DMSO-d₆) δ 10.80 (s, 1H), 9.39 (s, 1H), 9.38 (s, 1H), 8.70 (s, 1H), 8.34 (d, J=2.0 Hz, 1H), 8.29-8.19 (m, 1H), 7.75 (d, J=9.6 Hz, 1H), 7.68 (dd, J=9.6, 2.0 Hz, 1H), 3.83-3.76 (m, 4H), 3.20-3.12 (m, 4H), 2.12-2.01 (m, 1H), 1.34-1.23 (m, 1H), 1.12 (d, J=6.0 Hz, 3H), 1.04-0.95 (m, 1H), 0.88-0.80 (m, 1H).

### Example 573 and 574: Synthesis of (1R,2S)-2-(difluoromethyl)-N-(8-((methyl-d₃)amino)-5-(6-((S)-2-methylmorpholino)-[1,2,4]triazolo[1,5-a]pyridin-2-yl)-2,7-naphthyridin-3-yl)cyclopropane-1-carboxamide (Example 573) and (1S,2R)-2-(difluoromethyl)-N-(8-((methyl-d₃)amino)-5-(6-((S)-2-methylmorpholino)-[1,2,4]triazolo[1,5-a]pyridin-2-yl)-2,7-naphthyridin-3-yl)cyclopropane-1-carboxamide (Example 574)

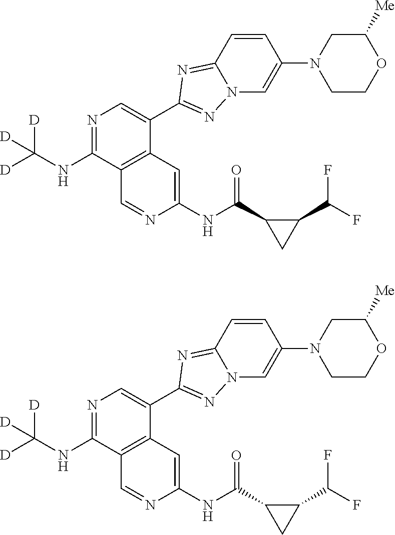 [SMILES: [2H]C([2H])([2H])Nc1ncc(-c2nc3ccc(N4CCO[C@@H](C)C4)cn3n2)c2cc(NC(=O)[C@@H]3C[C@@H]3C(F)F)ncc12 ; [2H]C([2H])([2H])Nc1ncc(-c2nc3ccc(N4CCO[C@@H](C)C4)cn3n2)c2cc(NC(=O)[C@H]3C[C@H]3C(F)F)ncc12]

### Step 1: (S)—N-(8-((methyl-d₃)amino)-5-(6-(2-methylmorpholino)-[1,2,4]triazolo[1,5-a]pyridin-2-yl)-2,7-naphthyridin-3-yl)cyclopropanecarboxamide

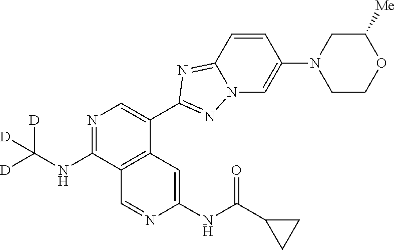 [SMILES: [2H]C([2H])([2H])Nc1ncc(-c2nc3ccc(N4CCO[C@@H](C)C4)cn3n2)c2cc(NC(=O)C3CC3)ncc12]

To a stirring mixture of XPhos Pd G₃ (440.0 mg; 0.520 mmol; 0.30 eq.), XPhos (250.0 mg; 0.521 mmol; 0.30 eq.) and K₃PO₄ (1.10 g; 5.211 mmol; 3.01 eq.) in a mixture solvent of 1,4-dioxane/water (5:1, 12 mL) were added (S)-4-(2-chloro-[1,2,4]triazolo[1,5-a]pyridin-6-yl)-2-methylmorpholine (440.0 mg; 1.743 mmol; 1.01 eq.) and N-(8-((methyl-d₃)amino)-5-(4,4,5,5-tetramethyl-1,3,2-dioxaborolan-2-yl)-2,7-naphthyridin-3-yl)cyclopropanecarboxamide (646.6 mg; 1.743 mmol; 1.00 eq.). The resulting solution was stirred at 11.0° C. for 2 hours under nitrogen atmosphere. The desired product was detected via LCMS. The mixture was concentrated under vacuum. The residue was purified by flash chromatography on silica gel column using 5-10% of MeOH: in CH₂Cl₂ as eluent to afford (S)—N-(8-((methyl-d₃)amino)-5-(6-(2-methylmorpholino)-[1,2,4]triazolo[1, 5-a]pyridin-2-yl)-2,7-naphthyridin-3-yl)cyclopropanecarboxamide as a brown solid (350.0 mg, 43.9%). LCMS (ESI) m/z 462.2, [M+H]⁺.

### Step 2: (S)—N¹-(methyl-d₃)-4-(6-(2-methylmorpholine)-[1,2,4]triazolo[1,5-a]pyridin-2-yl)-2,7-naphthyridine-1,6-diamine

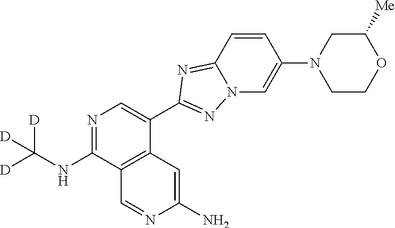 [SMILES: [2H]C([2H])([2H])Nc1ncc(-c2nc3ccc(N4CCO[C@@H](C)C4)cn3n2)c2cc(N)ncc12]

To a stirred solution of (S)—N-(8-((methyl-d₃)amino)-5-(6-(2-methylmorpholino)-[1,2,4]triazolo[1,5-a]pyridin-2-yl)-2,7-naphthyridin-3-yl)cyclopropanecarboxamide (350.0 mg; 0.76 mmol; 1.00 eq.) in MeOH (15 mL) was added a solution of NaOH (300.0 mg; 7.50 mmol; 9.89 eq.) in water (5 mL) at 0° C. The resulting solution was stirred at 60° C. for 12 hours. The desired product was detected via LCMS. The mixture was concentrated under vacuum. The residue was purified by flash chromatography on pre-packed C18 column using 50-70% of MeCN in water (10 mmol/L NH₄HCO₃) as eluent to provide (S)—N¹-(methyl-d₃)-4-(6-(2-methylmorpholino)-[1,2,4]triazolo[1,5-a]pyridin-2-yl)-2,7-naphthyridine-1,6-diamine as a yellow solid (250.0 mg, 83.8%). LCMS (ESI) m/z 394.2, [M+H]⁺.

### Step 3: (1R,2S)-2-(difluoromethyl)-N-(8-((methyl-d₃)amino)-5-(6-((S)-2-methylmorpholino)-[1,2,4]triazolo[1,5-a]pyridin-2-yl)-2,7-naphthyridin-3-yl)cyclopropane-1-carboxamide and (1S,2R)-2-(difluoromethyl)-N-(8-((methyl-d₃)amino)-5-(6-((S)-2-methylmorpholino)-[1,2,4]triazolo[1,5-a]pyridin-2-yl)-2,7-naphthyridin-3-yl)cyclopropane-1-carboxamide

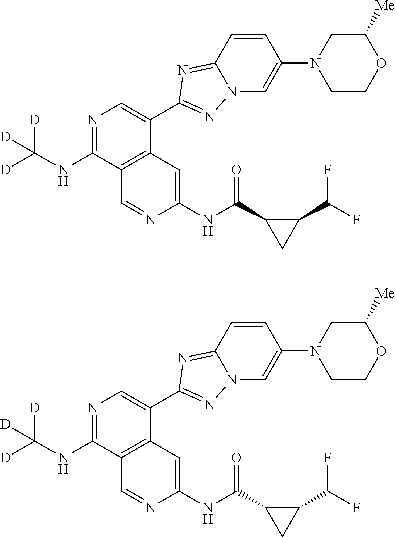 [SMILES: [2H]C([2H])([2H])Nc1ncc(-c2nc3ccc(N4CCO[C@@H](C)C4)cn3n2)c2cc(NC(=O)[C@@H]3C[C@@H]3C(F)F)ncc12 ; [2H]C([2H])([2H])Nc1ncc(-c2nc3ccc(N4CCO[C@@H](C)C4)cn3n2)c2cc(NC(=O)[C@H]3C[C@H]3C(F)F)ncc12]

To a stirred solution of (S)—N¹-(methyl-d₃)-4-(6-(2-methylmorpholino)-[1,2,4]triazolo[1,5-a]pyridin-2-yl)-2,7-naphthyridine-1,6-diamine (170.0 mg; 0.432 mmol; 1.00 eq.) and 2-(difluoromethyl)cyclopropane-1-carboxylic acid (cis racemate) (51.0 mg; 0.375 mmol; 0.87 eq.) in pyridine (10 mL) was added POCl₃ (198.0 mg; 1.291 mmol; 2.99 eq.) dropwise at 0° C. under nitrogen atmosphere. The resulting solution was stirred at room temperature for 0.5 hour. The reaction was quenched with water (1 mL) and concentrated under vacuum. The residue was purified by flash chromatography, on silica gel column, using 5-10% of MeOH in CH₂Cl₂ as eluent to afford the cis racemate. The cis racemic mixture was separated by Prep-Chiral-HPLC (Column: CHIRAL ART Cellulose-SC, 2×25 cm, 5 μm; Mobile Phase A: Hex (0.5% 2 M NH₃-MeOH), Mobile Phase B: EtOH: CH₂Cl₂=1:1; Flow rate: 20 mL/min; Gradient: 60% B to 60% B in 12.5 min) to afford (1R,2S)-2-(difluoromethyl)-N-(8-((methyl-d₃)amino)-5-(6-((S)-2-methylmorpholino)-[1,2,4]triazolo[1,5-a]pyridin-2-yl)-2,7-naphthyridin-3-yl)cyclopropane-1-carboxamide (Example 574, the faster peak) as a yellow solid (44.9 mg, 20.1%) and (1S,2R)-2-(difluoromethyl)-N-(8-((methyl-d₃)amino)-5-(6-((S)-2-methylmorpholino)-[1,2,4]triazolo[1,5-a]pyridin-2-yl)-2,7-naphthyridin-3-yl)cyclopropane-1-carboxamide (Example 574, the slower peak) as a yellow solid (45.5 mg, 20.6%). The two configurations are arbitrarily assigned. LCMS (ESI) m/z 512.2, [M+H]⁺.

HNMR for Example 573: ¹H NMR (400 MHz, DMSO-d₆) δ 11.16 (s, 1H), 9.43 (s, 1H), 9.41 (s, 1H), 8.70 (s, 1H), 8.34 (d, J=2.0 Hz, 1H), 8.31-8.28 (m, 1H), 7.75 (d, J=9.6 Hz, 1H), 7.69 (dd, J=9.6, 2.0 Hz, 1H), 6.20-5.90 (m, 1H), 4.00-3.90 (m, 1H), 3.82-3.60 (m, 3H), 3.58-3.50 (m, 1H), 2.78-2.71 (m, 1H), 2.46-2.39 (m, 2H), 1.99-1.86 (m, 1H), 1.39-1.86 (m, 2H), 1.18 (d, J=6.0 Hz, 3H).

HNMR for Example 574: ¹H NMR (400 MHz, DMSO-d₆) δ 11.16 (s, 1H), 9.43 (s, 1H), 9.41 (s, 1H), 8.70 (s, 1H), 8.34 (d, J=2.0 Hz, 1H), 8.31-8.28 (m, 1H), 7.75 (d, J=9.6 Hz, 1H), 7.69 (dd, J=9.6, 2.0 Hz, 1H), 6.20-5.90 (m, 1H), 4.00-3.90 (m, 1H), 3.82-3.60 (m, 3H), 3.58-3.50 (m, 1H), 2.78-2.71 (m, 1H), 2.46-2.39 (m, 2H), 1.99-1.86 (m, 1H), 1.39-1.86 (m, 2H), 1.18 (d, J=6.0 Hz, 3H).

### Example 576: Synthesis of (S)-1-fluoro-N-(8-((methyl-d₃)amino)-5-(6-(2-methylmorpholino)-[1,2,4]triazolo[1,5-a]pyridin-2-yl)-2,7-naphthyridin-3-yl)cyclopropane-1-carboxamide

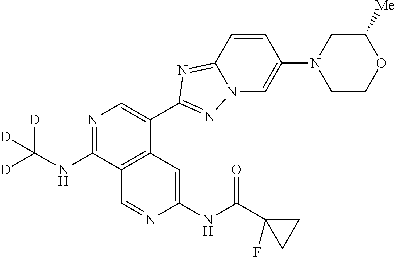 [SMILES: [2H]C([2H])([2H])Nc1ncc(-c2nc3ccc(N4CCO[C@@H](C)C4)cn3n2)c2cc(NC(=O)C3(F)CC3)ncc12]

To a stirred solution of (S)—N¹-(methyl-d₃)-4-(6-(2-methylmorpholino)-[1,2,4]triazolo[1,5-a]pyridin-2-yl)-2,7-naphthyridine-1,6-diamine (Example 574, Step 2) (76.0 mg; 0.19 mmol; 1.00 eq.) and 1-fluorocyclopropane-1-carboxylic acid (18.1 mg; 0.17 mmol; 0.90 eq.) in pyridine (3 mL) was added POCl₃ (88.7 mg; 0.58 mmol; 3.00 eq.) dropwise at 0° C. The reaction was stirred at room temperature for 0.5 hour under nitrogen atmosphere. The desired product was detected via LCMS. The mixture was diluted with EtOAc (90 mL), washed with brine (3×5 mL), dried with Na₂SO₄, and concentrated under reduced pressure. The residue was purified by flash chromatography on silica gel column using 2-10% of MeOH in CH₂Cl₂ as eluent to provide (S)-1-fluoro-N-(8-((methyl-d₃)amino)-5-(6-(2-methylmorpholino)-[1,2,4]triazolo[1,5-a]pyridin-2-yl)-2,7-naphthyridin-3-yl)cyclopropane-1-carboxamide as a white solid (27.1 mg, 29.2%). LCMS (ESI) m/z 480.2, [M+H]⁺. ¹H NMR (400 MHz, DMSO-d₆) δ 10.32 (s, 1H), 9.46 (s, 1H), 9.43 (s, 1H), 8.80 (s, 1H), 8.34 (d, J=2.0 Hz, 1H), 8.34-8.30 (m, 1H), 7.75 (d, J=9.6 Hz, 1H), 7.69 (dd, J=9.6, 2.0 Hz, 1H), 3.98-3.90 (m, 1H), 3.77-3.58 (m, 3H), 3.55-3.46 (m, 1H), 2.78-2.67 (m, 1H), 2.46-2.36 (m, 1H), 1.54-1.35 (m, 4H), 1.18 (d, J=6.0 Hz, 3H).

### Example 577: Synthesis of 1-fluoro-N-(8-((methyl-d₃)amino)-5-(6-methyl-[1,2,4]triazolo[1,5-a]pyridin-2-yl)-2,7-naphthyridin-3-yl)cyclopropane-1-carboxamide

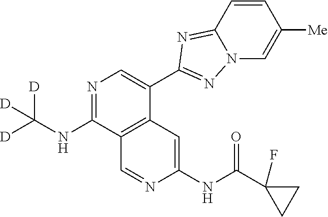 [SMILES: [2H]C([2H])([2H])Nc1ncc(-c2nc3ccc(C)cn3n2)c2cc(NC(=O)C3(F)CC3)ncc12]

### Step 1: N-(8-((methyl-d₃)amino)-5-(6-methyl-[1,2,4]triazolo[1,5-a]pyridin-2-yl)-2,7-naphthyridin-3-yl)cyclopropanecarboxamide

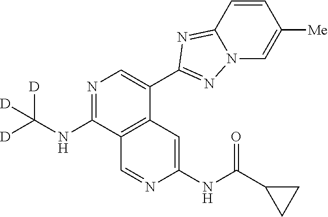 [SMILES: [2H]C([2H])([2H])Nc1ncc(-c2nc3ccc(C)cn3n2)c2cc(NC(=O)C3CC3)ncc12]

A stirred mixture of 2-chloro-6-methyl-[1,2,4]triazolo[1,5-a]pyridine (400.0 mg; 2.387 mmol; 1.00 eq.) and N-(8-((methyl-d₃)amino)-5-(4,4,5,5-tetramethyl-1,3,2-dioxaborolan-2-yl)-2,7-naphthyridin-3-yl)cyclopropanecarboxamide (1.15 g; 3.103 mmol; 1.30 eq.) in a mixture solvent of 1,4-dioxane/water (5:1, 12 mL). To the above mixture were added XPhos Pd G₃ (606.0 mg; 0.716 mmol; 0.30 eq.), XPhos (341.3 mg; 0.716 mmol; 0.30 eq.) and K₃PO₄ (1519.8 mg; 7.161 mmol; 3.00 eq.). The resulting mixture was stirred at 110° C. for 1 hour under nitrogen atmosphere. The desired product was detected via LCMS. The mixture was allowed to cool down to room temperature and the reaction mixture was concentrated under reduced pressure. The residue was purified by flash chromatography on silica gel column using 2-5% of MeOH in CH₂Cl₂ as eluent to provide N-(8-((methyl-d(3)amino)-5-(6-methyl-[1,2,4]triazolo[1,5-a]pyridin-2-yl)-2,7-naphthyridin-3-yl)cyclopropanecarboxamide as a light yellow solid (300.0 mg, 33.4%). LCMS (ESI) m/z 377.2, [M+H]⁺.

### Step 2: N¹-(methyl-d₃)-4-(6-methyl-[1,2,4]triazolo[1,5-a]pyridin-2-yl)-2,7-naphthyridine-1,6-diamine

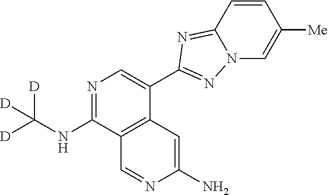 [SMILES: [2H]C([2H])([2H])Nc1ncc(-c2nc3ccc(C)cn3n2)c2cc(N)ncc12]

To a stirred solution of N-(8-((methyl-d₃)amino)-5-(6-methyl-[1,2,4]triazolo[1,5-a]pyridin-2-yl)-2,7-naphthyridin-3-yl)cyclopropanecarboxamide (300.0 mg; 0.797 mmol; 1.00 eq.) in a mixture solvent of MeOH/DMSO (3:5, 16 mL) was added a solution of Na₀₁₁(318.8 mg; 7.970 mmol; 10.00 eq.) in water (2 mL) at 0° C. The mixture was stirred at 60° C. overnight. The desired product was detected via LCMS. The mixture was allowed to cool down to room temperature. The mixture was concentrated under reduced pressure. The residue was purified by flash chromatography on pre-packed C18 column using 20-60% of MeOH in water (10 mmol/L NH₄HCO₃) as eluent to provide N¹-(methyl-d₃)-4-(6-methyl-[1,2,4]triazolo[1,5-a]pyridin-2-yl)-2,7-naphthyridine-1,6-diamine as a yellow solid (150.0 mg, 61.0%). LCMS (ESI) m/z 309.2, [M+H]⁺.

### Step 3: 1-fluoro-N-(8-((methyl-d₃)amino)-5-(6-methyl-[1,2,4]triazolo[1,5-a]pyridin-2-yl)-2,7-naphthyridin-3-yl)cyclopropane-1-carboxamide

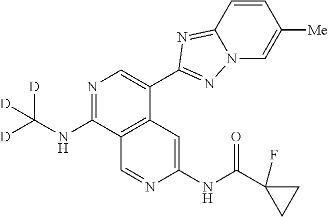 [SMILES: [2H]C([2H])([2H])Nc1ncc(-c2nc3ccc(C)cn3n2)c2cc(NC(=O)C3(F)CC3)ncc12]

To a stirred solution of N¹-(methyl-d₃)-4-(6-methyl-[1,2,4]triazolo[1,5-a]pyridin-2-yl)-2,7-naphthyridine-1,6-diamine (150.0 mg; 0.486 mmol; 1.00 eq.) and 1-fluorocyclopropane-1-carboxylic acid (35.6 mg; 0.342 mmol; 0.90 eq.) in pyridine (5 mL) was added POCl₃ (174.9 mg; 1.140 mmol; 3.00 eq.) dropwise at 0° C. The resulting mixture was stirred at room temperature for 0.5 hour under nitrogen atmosphere. The desired product was detected via LCMS. The reaction was quenched by water (5 mL) and extracted with EtOAc (3×10 mL). The combined organic layers were washed with brine (3×5 mL) and dried over anhydrous Na₂SO₄. After filtration, the filtrate was concentrated under reduced pressure. The residue was purified by flash chromatography on silica gel column using 5-8% of MeOH in CH₂Cl₂ as eluent to provide the crude product. The crude product was purified by recrystallization with a mixture solvent of CH₂Cl₂/MeOH (10:1, 5 mL) to afford 1-fluoro-N-(8-((methyl-d₃)amino)-5-(6-methyl-[1,2,4]triazolo[1,5-a]pyridin-2-yl)-2,7-naphthyridin-3-yl)cyclopropane-1-carboxamide as an off-white solid (100.8 mg, 66.8%). LCMS (ESI) m/z 395.2, [M+H]⁺. ¹H NMR (400 MHz, DMSO-d₆) δ 10.31 (s, 1H), 9.54 (s, 1H), 9.46 (s, 1H), 8.87 (s, 1H), 8.80 (d, J=2.0 Hz, 1H), 8.38-8.30 (m, 1H), 7.76 (d, J=9.6 Hz, 1H), 7.55 (dd, J=9.6, 2.0 Hz, 1H), 2.41 (s, 3H), 1.54-1.33 (m, 4H).

### Example 578: Synthesis of (1S,2S)-2-fluoro-N-(8-((methyl-d₃)amino)-5-(6-methyl-[1,2,4]triazolo[1,5-a]pyridin-2-yl)-2,7-naphthyridin-3-yl)cyclopropane-1-carboxamide

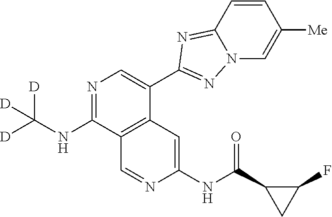 [SMILES: [2H]C([2H])([2H])Nc1ncc(-c2nc3ccc(C)cn3n2)c2cc(NC(=O)[C@@H]3C[C@@H]3F)ncc12]

To a stirred solution of N¹-(methyl-d₃)-4-(6-methyl-[1,2,4]triazolo[1,5-a]pyridin-2-yl)-2,7-naphthyridine-1,6-diamine (Example 577, Step 2) (70.0 mg; 0.227 mmol; 1 eq.) and (1S,2S)-2-fluorocyclopropane-1-carboxylic acid (18.9 mg; 0.182 mmol; 0.80 eq.) in pyridine (2 mL) was added POCl₃ (104.4 mg; 0.681 mmol; 3.00 eq.) dropwise at 0° C. The resulting mixture was stirred at room temperature for 0.5 hour under nitrogen atmosphere. The desired product was detected via LCMS. The reaction was quenched by the addition of water (10 mL) and extracted with EtOAc (3×20 mL). The combined organic layers were washed with brine (3×10 mL), dried over anhydrous Na₂SO₄. After filtration, the filtrate was concentrated under reduced pressure. The residue was purified by flash chromatography on silica gel column using 5-8% of MeOH in CH₂Cl₂ as eluent to provide the crude product. The crude product was purified by recrystallization with a mixture solvent of MeOH/CH₂Cl₂ (1:10, 5 mL) to afford (1S,2S)-2-fluoro-N-(8-((methyl-d₃)amino)-5-(6-methyl-[1,2,4]triazolo[1,5-a]pyridin-2-yl)-2,7-naphthyridin-3-yl)cyclopropane-1-carboxamide as a white solid (28.6 mg, 31.3%). LCMS (ESI) m/z 395.2, [M+H]⁺. ¹H NMR (400 MHz, DMSO-d₆) δ 10.98 (s, 1H), 9.49 (s, 1H), 9.42 (s, 1H), 8.80 (s, 1H), 8.79 (d, J=2.0 Hz, 1H), 8.29-8.23 (m, 1H), 7.77 (d, J=9.6 Hz, 1H), 7.55 (dd, 0.1=9.6, 2.0 Hz, 1H), 5.09-4.80 (m, 1H), 2.42 (s, 3H), 2.34-2.22 (m, 1H), 1.77-1.62 (m, 1H), 1.26-1.1.3 (m, 1H).

### Example 579: Synthesis of (1S,2R)—N-(8-(ethylamino)-5-(6-morpholino-[1,2,4]triazolo[1,5-a]pyridin-2-yl)-2,7-naphthyridin-3-yl)-2-methylcyclopropane-1-carboxamide

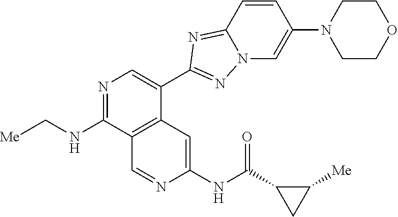 [SMILES: CCNc1ncc(-c2nc3ccc(N4CCOCC4)cn3n2)c2cc(NC(=O)[C@H]3C[C@H]3C)ncc12]

### Step 1: N¹-ethyl-4-(6-morpholino-[1,2,4]triazolo[1-5-a]pyridin-2-yl)-2,7-naphthyridine-1,6-diamine

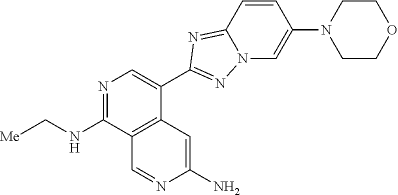 [SMILES: CCNc1ncc(-c2nc3ccc(N4CCOCC4)cn3n2)c2cc(N)ncc12]

To a solution of N-(8-(ethylamino)-5-(6-morpholino-[1,2,4]triazolo[1,5-a]pyridin-2-yl)-2,7-naphthyridin-3-yl)cyclopropanecarboxamide (Example 514) (250 mg; 0.545 mmol; 1.00 eq.) in a mixture solvent of DMSO/MeOH (1:5, 18 mL) was added a solution of NaOH (218.1 mg; 5.453 mmol; 10.00 eq.) in water (4.5 mL) dropwise at 0° C. The resulting mixture was stirred at 60° C. overnight. LC-MS indicated that the reaction was complete. The resulting mixture was concentrated under reduced pressure. The residue was purified by flash chromatography on pre-packed C18 column using 30-50% of MeOH in water (10 mmol/L NH₄HCO₃) as eluent to provide N¹-ethyl-4-(6-morpholino-[1,2,4]triazolo[1,5-a]pyridin-2-yl)-2,7-naphthyridine-1,6-diamine as a light brown solid (170.0 mg, 79.8%). LCMS (ESI) m/z 391.2, [M+H]⁺.

### Step 2: (1S,2R)—N-(8-(ethylamino)-5-(6-morpholino-[1,2,4]triazolo[1,5-a]pyridin-2-yl)-2,7-naphthyridin-3-yl)-2-methylcyclopropane-1-carboxamide

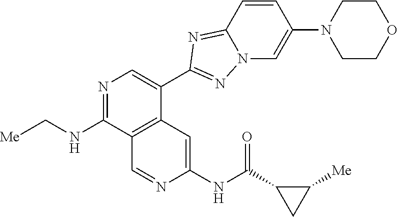 [SMILES: CCNc1ncc(-c2nc3ccc(N4CCOCC4)cn3n2)c2cc(NC(=O)[C@H]3C[C@H]3C)ncc12]

To a stirred solution of N¹-ethyl-4-(6-morpholino-[1,2,4]triazolo[1,5-a]pyridin-2-yl)-2,7-naphthyridine-1,6-diamine (70.0 mg; 0.179 mmol; 1.00 eq.) and (1S,2R)-2-methylcyclopropane-1-carboxylic acid (16.2 mg; 0.162 mmol; 0.90 eq.) in pyridine (3 mL) was added POCl₃ (82.5 mg; 0.538 mmol; 3.00 eq.) dropwise at 0° C. The resulting mixture was stirred at room temperature for 0.5 hour under nitrogen atmosphere. The desired product was detected via LCMS. The reaction was quenched by the addition of water (1 mL) at 0° C. The resulting mixture was concentrated under reduced pressure. The residue was purified by flash chromatography on silica gel column using 1-6% of MeOH in CH₂Cl₂ as eluent to provide the crude product. The product was purified by flash chromatography on pre-packed C18 column using 50-80% of MeOH in water (10 mmol/L NH₄HCO₃) as eluent to provide (1S,2R)—N-(8 (ethylamino)-5-(6-morpholino-[1,2,4]triazolo[1,5-a]pyridin-2-yl)-2,7-naphthyridin-3-yl)-2-methylcyclopropane-1-carboxamide as an off-white solid (31.2 mg, 36.8%). LCMS (ESI) m/z 473.2, [M+H]⁺. ¹H NMR (400 MHz, DMSO-d₆) δ 10.81 (s, 1H), 9.44 (s, 1H), 9.37 (s, 1H), 8.69 (s, 1H), 8.34 (d, J=2.0 Hz, 1H), 8.18-8.15 (m, 1H), 7.75 (d, J=9.6 Hz, 1H), 7.69 (dd, J=9.6, 2.0 Hz, 1H), 3.82-3.76 (m, 4H), 3.64-3.58 (m, 2H), 3.19-3.14 (m, 4H), 2.12-2.04 (m, 1H), 1.31-1.24 (m, 4H), 1.13 (d, J=6.0 Hz, 3H), 1.01-0.95 (m, 1H), 0.86-0.80 (m, 1H).

### Example 580: Synthesis of N-(8-(ethylamino)-5-(6-morpholino-[1,2,4]triazolo[1,5-a]pyridin-2-yl)-2,7-naphthyridin-3-yl)-1-fluorocyclopropane-1-carboxamide

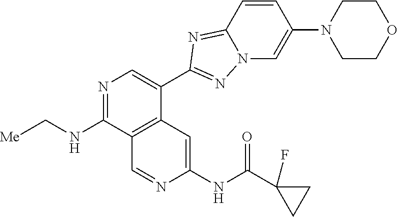 [SMILES: CCNc1ncc(-c2nc3ccc(N4CCOCC4)cn3n2)c2cc(NC(=O)C3(F)CC3)ncc12]

To a stirred solution of N¹-ethyl-4-(6-morpholino-[1,2,4]triazolo[1,5-a]pyridin-2-yl)-2,7-naphthyridine-1,6-diamine (Example 579, Step 1) (75.0 mg; 0.192 mmol; 1.00 eq.) and 1-fluorocyclopropane-1-carboxylic acid (17.9 mg; 0.172 mmol; 0.90 eq.) in pyridine (3 mL) was added POCl₃ (88.3 mg; 0.576 mmol; 3.00 eq.) dropwise at 0′C. The resulting mixture was stirred at room temperature for 0.5 hour under nitrogen atmosphere. The desired product was detected via LCMS. The reaction was quenched by the addition of water (1 mL) at 0° C. The resulting mixture was concentrated under reduced pressure. The residue was purified by flash chromatography on silica gel column using 1-6% of MeOH in CH₂Cl₂ as eluent to provide the crude product. The crude product was purified by flash chromatography on pre-packed C18 column using 50-80% of MeOH in water (1.0 mmol/L NH₄HCO₃) as eluent to provide N-(8-(ethylamino)-5-(6-morpholino-[1,2,4]triazolo[1,5-a]pyridin-2-yl)-2,7-naphthyridin-3-yl)-1-fluorocyclopropane-1-carboxamide as a white solid (47.3 mg, 51.6%). LCMS (ESI) m/z 477.2, [M+H]⁺. . ¹H NMR (400 MHz, DMSO-d₆) δ 10.31 (s, 1H), 9.50 (s, 1H), 9.43 (s, 1H), 8.78 (s, 1H), 8.35 (d, J=2.0 Hz, 1H), 8.30-8.25 (m, 1H), 7.75 (d, J=9.6 Hz, 1H), 7.68 (dd, J=9.6, 2.0 Hz, 1H), 3.84-3.76 (m, 4H), 3.68-3.58 (m, 2H), 3.17-3.10 (m, 4H), 1.49-1.36 (m, 4H), 1.27 (t, J=7.2 Hz, 3H).

### Example 581: Synthesis of N-(8-(ethylamino)-5-(6-(7-methyl-9-oxa-3,7-diazabicyclo[3.3.1]nonan-3-yl)-[1,2,4]triazolo[1,5-a]pyridin-2-yl)-2,7-naphthyridin-3-yl)cyclopropanecarboxamide

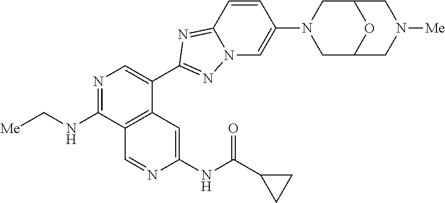 [SMILES: CCNc1ncc(-c2nc3ccc(N4CC5CN(C)CC(C4)O5)cn3n2)c2cc(NC(=O)C3CC3)ncc12]

### Step 1: tert-butyl 7-(2-chloro-[1,2,4]triazolo[1,5-a]pyridin-6-yl)-9-oxa-3,7-diazabicyclo[3.3.1]nonane-3-carboxylate

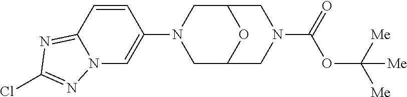 [SMILES: CC(C)(C)OC(=O)N1CC2CN(c3ccc4nc(Cl)nn4c3)CC(C1)O2]

A stirring mixture of 6-bromo-2-chloro-[1,2,4]triazolo[1,5-a]pyridine (700.0 mg; 3.010 mmol; 1.00 eq.), tert-butyl 9-oxa-3,7-diazabicyclo[3.3.1]nonane-3-carboxylate (687.4 mg; 3.010 mmol; 1.00 eq.), Pd-PEPPSI-IHeptCl 3-chloropyridine (293.3 mg; 0.301 mmol; 0.10 eq.) and Cs₂CO₃ (3.92 g; 12.040 mmol; 4.00 eq.) in 1,4-dioxane (28 mL) was stirred at 100° C. overnight under nitrogen atmosphere. The desired product was detected via LCMS. The resulting mixture was concentrated under reduced pressure. The residue was purified by flash chromatography on silica gel column using 30-70% of EtOAc in petroleum ether as eluent to provide tort-butyl 7-(2-chloro-[1,2,4]triazolo[1,5-a]pyridin-6-yl)-9-oxa-3,7-diazabicyclo[3.3.1]nonane-3-carboxylate as a yellow solid (828.7 mg, 71.0%). LCMS (ESI) m/z 380.1, [M+H]⁺.

### Step 2: 3-(2-chloro-[1,2,4]triazolo[1,5-a]pyridin-6-yl)-9-oxa-3,7-diazabicyclo[3.3.1]nonane hydrochloride

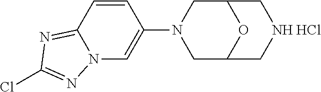

To a stirring mixture of tert-butyl 7-(2-chloro-[1,2,4]triazolo[1,5-a]pyridin-6-yl)-9-oxa-3,7-diazabicyclo[3.3.1]nonane-3-carboxylate (520.0 mg; 1.368 mmol; 1.00 eq.) in MeOH (10 mL) was added HCl (gas) (4 M in 1,4-dioxane, 4 mL). The resulting solution was stirred at room temperature for 2 hours. The desired product was detected via LCMS. The resulting mixture was concentrated under reduced pressure to afford 3-(2-chloro-[1,2,4]triazolo[1,5-a]pyridin-6-yl)-9-oxa-3,7-diazabicyclo[3.3.1]nonane hydrochloride as a colorless oil (500.0 mg, crude). LCMS (ESI) m/z 280.1, [M+H]⁺.

### Step 3: 3-(2-chloro-[1,2,4]triazolo[1,5-a]pyridin-6-yl)-7-methyl-9-oxa-3,7-diazabicyclo[3.3.1]nonane

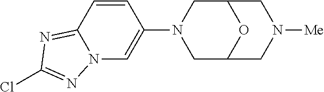 [SMILES: CN1CC2CN(c3ccc4nc(Cl)nn4c3)CC(C1)O2]

To a solution of 3-(2-chloro-[1,2,4]triazolo[1,5-a]pyridin-6-yl)-9-oxa-3,7-diazabicyclo[3.3.1]nonane hydrochloride (500.0 mg; 1.581 mmol; 1.00 eq.), HCHO (35% w/w in water, 1.35 g) and DIEA (694.9 mg; 5.377 mmol; 3.40 eq.) in dichloroethane/MeOH (4:1, 25 mL) was added NaBH(OAc)₃ (1.14 g; 5.379 mmol; 3.40 eq.). The mixture was stirred at room temperature for 1 hour under nitrogen atmosphere. The desired product was detected via LCMS. The mixture was concentrated under reduced pressure. The residue was purified by flash chromatography on pre-packed C18 column using 20-45% of ACN in water (1.0 mmol/L NH₄HCO₃) as eluent to provide 3-(2-chloro-[1,2,4]triazolo[1,5-a]pyridin-6-yl)-7-methyl-9-oxa-3,7-diazabicyclo[3.3.1]nonane as a white solid (350.0 mg, 75.3%). LCMS (ESI) m/z 294.1, [M+H]⁺.

### Step 4: N-(8-(ethylimino)-5(6-(7-methyl-9-oxa-3,7-diazabicyclo[3.3.1]nonan-3-yl)-[1,2,4]triazolo[1,5-a]pyridin-2-yl)-2,7-naphthyridin-3-yl)cyclopropanecarboxamide

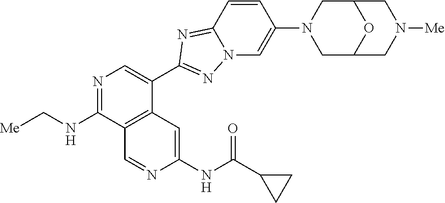

A suspension of 3-(2-chloro-[1,2,4]triazolo[1,5-a]pyridin-6-yl)-7-methyl-9-oxa-3,7-diazabicyclo[3.3.1]nonane (70.0 mg; 0.238 mmol; 1.00 eq.), N-(8-(ethylamino)-5-(4,4,5,5-tetramethyl-1,3,2-dioxaborolan-2-yl)-2,7-naphthyridin-3-yl)cyclopropanecarboxamide (91.0 mg; 0.24 mmol; 1.00 eq.), XPhos Pd G₃ (60.5 mg; 0.072 mmol; 0.30 eq.), XPhos (34.1 mg; 0.072 mmol; 0.30 eq.) and K₃PO₄ (151.7 mg; 0.72 mmol; 3.00 eq.) in 1,4-dioxane/water (5:1, 8.4 mL) was stirred at 110° C. for 1 hour under nitrogen atmosphere. Upon completion, the resulting mixture was concentrated under reduced pressure. The residue was purified by flash chromatography on silica gel column using 0-10% of MeOH in CH₂Cl₂ (0.1% Et₃N) as eluent to afford the crude product. This crude product was recrystallized by a mixed solvent of hexane/MeOH (5:1, 10 mL) to provide N-(8-(ethylamino)-5-(6-(7-methyl-9-oxa-3,7-diazabicyclo[3.3.1]nonan-3-yl)-[1,2,4]triazolo[1,5-a]pyridin-2-yl)-2,7-naphthyridin-3-yl)cyclopropanecarboxamide as an off-white solid (31.7 mg, 25.9%). LCMS (ESI) m/z 514.3, [M+H]⁺. ¹H NMR (400 MHz, DMSO-d₆) δ 10.93 (s, 1H), 9.44 (s, 1H), 9.42 (s, 1H), 8.71 (s, 1H).

### Example 582: Synthesis of N-(5-(6-(7-cyclopropyl-9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide

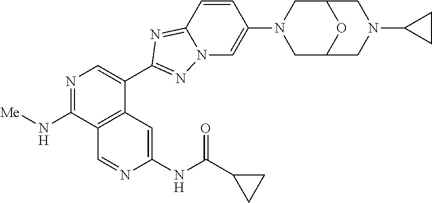

### Step 1: 3-(2-chloro-[1,2,4]triazolo[1,5-a]pyridin-6-yl)-7-cyclopropyl-9-oxa-3,7-diazabicyclo[3.3.1]nonane

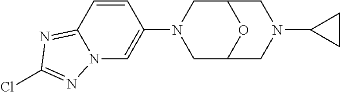 [SMILES: Clc1nc2ccc(N3CC4CN(C5CC5)CC(C3)O4)cn2n1]

A suspension of 3-(2-chloro-[1,2,4]triazolo[1,5-a]pyridin-6-yl)-9-oxa-3,7-diazabicyclo[3.3.1.]nonane hydrochloride (Example 581, Step 2) (170.0 mg; 0.538 mmol; 1.00 eq.), cyclopropylboronic acid (92.3 mg; 1.076 mmol; 2.00 eq.), Cu(OAc)₂ (97.6 mg; 0.538 mmol; 1.00 eq.), 2,2′-bipyridine (83.9 mg; 0.538 mmol; 1.00 eq.) and Na₂CO₃ (113.9 mg; 1.075 mmol; 2.00 eq.) in dichloroethane (12 mL) was stirred at 70° C. for 2 hours under oxygen atmosphere. The desired product was detected via LCMS. The resulting mixture was concentrated under reduced pressure. The residue was purified by flash chromatography on silica gel column using 10-30% of EtOAc in petroleum ether as eluent to provide 3-(2-chloro-[1,2,4]triazolo[1,5-a]pyridin-6-yl)-7-cyclopropyl-9-oxa-3,7-diazabicyclo[3.3.1]nonane as a yellow solid (102.9 mg, 58.8%). LCMS (ESI) m/z 320.1, [M+H]⁺.

### Step 2: N-(5-(6-(7-cyclopropyl-9-oxa-3,7-diazabicyclo[3.3.1]nonan-3-yl)-[1,2,4]triazolo[1,5-a]pyridin-2-yl)-8-(methylamino)-2,7-naphthyridin-3-yl)cyclopropanecarboxamide

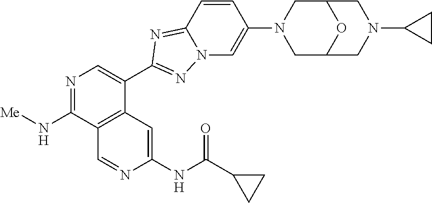

A stirring mixture of N-(8-(methyl amino)-5-(4,4,5,5-tetramethyl-1,3,2-dioxaborolan-2-yl)-2,7-naphthyridin-3-yl)cyclopropanecarboxamide (100.0 mg; 0.272 mmol; 1.00 eq.), 3-(2-chloro-[1,2,4]triazolo[1,5-a]pyridin-6-yl)-7-cyclopropyl-9-oxa-3,7-diazabicyclo[3.3.1]nonane (86.8 mg; 0.271 mmol; 1.00 eq.), XPhos Pd G₃ (68.9 mg; 0.081 mmol; 0.30 eq.), XPhos (38.8 mg; 0.081 mmol; 0.30 eq.) and K₃PO₄ (1.729 mg; 0.81 mmol; 3.00 eq.) in 1,4-dioxane/water (5:1, 12 mL) was stirred at 110° C. for 1 hour under nitrogen atmosphere. LC-MS indicated that the starting material was consumed. The resulting mixture was concentrated under reduced pressure. The residue was purified by flash chromatography on silica gel column using 0-10% of MeOH in CH₂Cl₂ as eluent to provide N-(5-(6-(7-cyclopropyl-9-oxa-3,7-diazabicyclo[3.3.1]nonan-3-yl)-[1,2,4]triazolo[1, 5-a]pyridin-2-yl)-8-(methyl amino)-2,7-naphthyridin-3-yl)cyclopropanecarboxamide as a light yellow solid (51.4 mg, 36.0%). LCMS (ESI) m/z 526.3, ¹H NMR (400 MHz, DMSO-d₆) δ 10.90 (s, 1H), 9.43 (s, 1H), 9.40 (s, 1H), 8.72 (s, 1H), 8.29-8.15 (m, 1H), 8.11 (d, J=1.6 Hz, 1H), 7.66 (d, J=9.6 Hz, 1H), 7.53 (dd, J=9.6, 2.4 Hz, 1H), 4.01-3.96 (m, 2H), 3.67 d, J=1.1.2 Hz, 2H), 3.27-3.18 (m, 2H), 3.04 (d, J=4.4 Hz, 3H), 2.97 (d, J=11.2 Hz, 2H), 2.68-2.61 (m, 2H), 2.13-2.03 (m, 1H), 1.48-1.39 (m, 1H), 0.92-0.80 (m, 4H), 0.32-0.23 (m, 2H), 0.10-0.02 (m, 2H).

### Example 583: Synthesis of N-(5-(6-(3-oxa-7-azabicyclo[3.3.1]nonan-7-yl)-[1,2,4]triazolo[1,5-a]pyridin-2-yl)-8-(methylamino)-2,7-naphthyridin-3-yl)cyclopropanecarboxamide

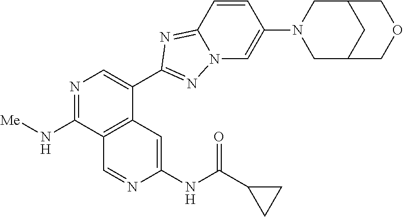

### Step 1: 7-(2-chloro-[1,2,4]triazolo[1,5-a]pyridin-6-yl)-3-oxa-7-azabicyclo[3.3.1]nonane

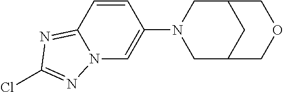

A stirring mixture of 6-bromo-2-chloro-[1,2,4]triazolo[1,5-a]pyridine (200.0 mg; 0.860 mmol; 1.00 eq.), Pd-PEPPSI-IHeptCl 3-chloropyridine (83.7 mg; 0.086 mmol; 0.10 eq.), Cs₂CO₃ (560.6 mg; 1.72 mmol; 2.00 eq.) and 3-oxa-7-azabicyclo[3.3.1]nonane (109.4 mg; 0.860 mmol; 1.00 eq.) in 1,4-dioxane (4 mL) was stirred at 100° C. overnight under nitrogen atmosphere. After the reaction was completed, the mixture was concentrated under reduced pressure. The residue was purified by flash chromatography on silica gel column using 0-10% of MeOH in CH₂Cl₂ as eluent to provide 7-(2-chloro-[1,2,4]triazolo[1,5-a]pyridin-6-yl)-3-oxa-7-azabicyclo[3.3.1]nonane as a yellow solid (120.1 mg, 50.0%). LCMS (ESI) m/z 279.1, [M+H]⁺.

### Step 2: N-(5-(6-(3-oxa-7-azabicyclo[3.3.1]nonan-7-yl)-[1,2,4]triazolo[1,5-a]pyridin-2-yl)-8-(methylamino)-2,7-naphthyridin-3-yl)cyclopropanecarboxamide

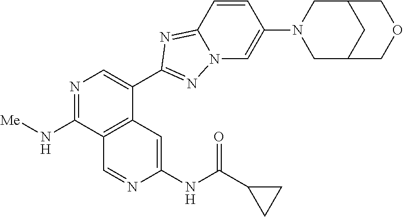

A stirring mixture of 7-(2-chloro-[1,2,4]triazolo[1,5-a]pyridin-6-yl)-3-oxa-7-azabicyclo[3.3.1]nonane (75.7 mg; 0.272 mmol; 1.00 eq.), XPhos (38.8 mg; 0.082 mmol; 0.30 eq.), XPhos Pd G₃ (68.9 mg; 0.082 mmol; 0.30 eq.), K₃PO₄ (172.9 mg; 0.816 mmol; 3.00 eq.) and N-(8-(methylamino)-5-(4,4,5,5-tetramethyl-1,3,2-dioxaborolan-2-yl)-2,7-naphthyridin-3-yl)cyclopropanecarboxamide (100.0 mg; 0.272 mmol; 1.00 eq.) in 1,4-dioxane/water (5:1, 2.4 mL) was stirred at 110° C. for 1 hour under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide N-(5-(6-(3-oxa-7-azabicyclo[3.3.1]nonan-7-yl)-[1,2,4]triazolo[1,5-a]pyridin-2-yl)-8-(methylamino)-2,7-naphthyridin-3-yl)cyclopropanecarboxamide as a yellow solid (42.1 mg, 31.9%). LCMS (ESI) m/z 485.2, [M+H]⁺. ¹H NMR (400 MHz, DMSO-d₆) δ 10.92 (s, 1H), 9.44 (s, 1H), 9.40 (s, 1H), 8.74 (s, 1H), 8.28-8.19 (m, 1H), 8.12 (d, J=2.4 Hz, 1H), 7.74-7.64 (m, 2H), 3.91 (d, J=11.2 Hz, 2H), 3.79 (d, J=11.2 Hz, 2H), 3.68 (d, J=11.2 Hz, 2H), 3.09-2.99 (m, 5H), 2.11-2.01 (m, 1H), 1.96-1.89 (m, 3H), 1.84-1.74 (m, 1H), 0.90-0.80 (m, 4H).

### Example 584: Synthesis of N-(5-(6-(8-oxa-3-azabicyclo[3.2.1]octan-3-yl)-[1,2,4]triazolo[1,5-a]pyridin-2-yl)-8-(methylamino)-2,7-naphthyridin-3-yl)cyclopropanecarboxamide

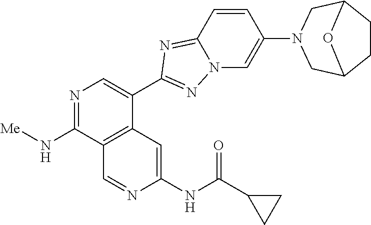

### Step 1: 3-(2-chloro-[1,2,4]triazolo[1,5-a]pyridin-6-yl)-8-oxa-3-azabicyclo[3.2.1]octane

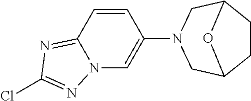

A stirring mixture of 6-bromo-2-chloro-[1,2,4]triazolo[1,5-a]pyridine (200.0 mg; 0.860 mmol; 1.00 eq.), Pd-PEPPSI-IHeptCl 3-chloropyridine (83.7 mg; 0.086 mmol; 0.10 eq.), Cs₂CO₃ (560.6 mg; 1.720 mmol; 2.00 eq.) and 8-oxa-3-azabicyclo[3.2.1]octane hydrochloride (128.7 mg; 0.860 mmol; 1.00 eq.) in 1,4-dioxane (4 mL) was stirred at 100° C. overnight under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide 3-(2-chloro-[1,2,4]triazolo[1,5-a]pyridin-6-yl)-8-oxa-3-azabicyclo[3.2.1]octane as an off-white solid (150.0 mg, 65.8%). LCMS (ESI) m/z 265.1, [M+H]⁺.

### Step 2: PV-(5-(6-(8-oxa-3-azabicyclo[3.2.1]octan-3-yl)[1,2,4]triazolo[1,5-a]pyridin-2-yl)-8-(methylamino)-2,7-naphthyridin-3-yl)cyclopropancarboxamide

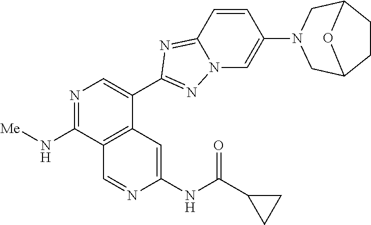

A mixture of 3-chloro-[1,2,4]triazolo[1,5-a]pyridin-6-0)-8-oxa-3-azabicyclo[3.2.1]octane (71.9 mg; 0.272 mmol; 1.00 eq.), XPhos (38.8 mg, 0.082 mmol; 0.30 eq.), XPhos Pd (13 (68.9 mg; 0.082 mmol; 0.30 eq.), K₃PO₁ (172.9 mg; 0.816 mmol, 3.00 eq.) and N-(8-(methylamino)-5-(4,4,5,5-tetramethyl-1,3,2-dioxaborolan-2-yl)-2,7-naphthyridin-3-yl)cyclopropanecarboxamide (100.0 mg; 0.272 mmol; 1.00 eq.) in 1,4-dioxane/water (5:1, 2.4 mL) was stirred 110° C. for 1 hour under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide N-(5-(6-(8-oxa-3-azabicyclo[3.2.1]octan-3-yl)-[1,2,4]triazolo[1,5-a]pyridin-2-yl)-8-(methylamino)-2,7-naphthyridin-3-yl)cyclopropanecarboxamide as a yellow solid (28.8 mg, 22.3%). LCMS (ESI) m/z 471.2, [M+H]⁺. ¹H NMR (400 MHz, DMSO-d₆) δ 10.91 (s, 1H), 9.41 (s, 1H), 9.40 (s, 1H), 8.73 (s, 1H), 8.32-8.00 (m, 2H), 7.79-7.49 (m, 2H), 4.60-4.32 (m, 2H), 3.48-3.39 (m, 2H), 3.05 (d, J=4.4 Hz, 3H), 2.98-2.82 (m, 2H), 2.1.1-2.03 (m, 1H), 1.98-1.82 (m, 4H), 0.89-0.80 (m, 4H).

### Example 585 and 586: Synthesis of (1S,2S)-2-ethyl-N-(8-(methylamino)-5-(6-morpholino-[1,2,4]triazolo[1,5-a]pyridin-2-yl)-2,7-naphthyridin-3-yl)cyclopropane-1-carboxamide (Example 585) and (1R,2R)-2-ethyl-N-(8-(methylamino)-5-(6-morpholino-[1,2,4]triazolo[1,5-a]pyridin-2-yl)-2,7-naphthyridin-3-yl)cyclopropane-1-carboxamide (Example 586)

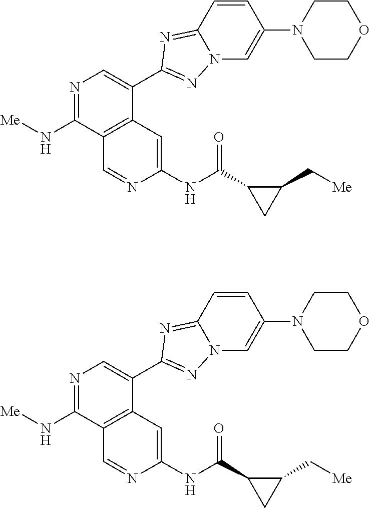

To a solution of N¹-methyl-4-(6-morpholino-[1,2,4]triazolo[1,5-a]pyridin-2-yl)-2,7-naphthyridine-1,6-diamine (Example 525, Step 1) (120.0 mg; 0.32 mmol; 1.00 eq.) and 2-ethylcyclopropane-1-carboxylic acid (trans racemate) (32.7 mg; 0.29 mmol; 0.90 eq.) in pyridine (3 mL) was added POCl₃ (146.5 mg; 0.96 mmol; 3.00 eq.) dropwise at 0° C. The resulting mixture was stirred at room temperature for 0.5 hour under nitrogen atmosphere. LC-MS indicated that the reaction was complete. The reaction was quenched by the addition of water (5 mL) and extracted with EtOAc (3×30 mL). The combined organic layers were washed with brine (3×10 mL), dried over anhydrous Na₂SO₄, filtered, and concentrated under reduced pressure. The residue was purified by flash chromatography on silica gel column using 0-10% of MeOH in CH₂Cl₂ as eluent to provide the trans racemate. The trans racemate was separated by Prep-Chiral-HPLC (Column: Lux 5 um Cellulose-2 2.12×25 cm, 5 μm; Mobile Phase A: Hex(0.5% 2 M NH₃-MeOH), Mobile Phase B: MeOH:EtOH=1:1; Flow rate: 20 mL/min; Gradient: 70% B to 70% B in 15 min; Wave Length: 220/254 nm; R_{T1}(min): 8.65; R_{T2}(min): 11.62) to afford (1S,2S)-2-ethyl-N-(8-(methylamino)-5-(6-morpholino-[1,2,4]triazolo[1,5-a]pyridin-2-yl)-2,7-naphthyridin-3-yl)cyclopropane-1-carboxamide (Example 585, the faster peak) as a yellow solid (33.5 mg, 22.2%) and (1R,2R)-2-ethyl-N-(8-(methylamino)-5-(6-morpholino-[1,2,4]triazolo[1,5-a]pyridin-2-yl)-2,7-naphthyridin-3-yl)cyclopropane-1-carboxamide (Example 586, the slower peak) as a yellow solid (36.9 mg, 24.4%). The two configurations are arbitrarily assigned. LCMS (ESI) m/z 473.2, [M+H]⁺.

HNMR for Example 585: ¹H NMR (400 MHz, DMSO-d₆) δ 10.85 (s, 1H), 9.40 (s, 1H), 9.39 (s, 1H), 8.72 (s, 1H), 8.34 (d, J=1.6 Hz, 1H), 8.30-8.29 (m, 1H), 7.79-7.67 (m, 2H), 3.84-3.76 (m, 4H), 3.21-3.13 (m, 4H), 3.05 (d, J=4.4 Hz, 3H), 1.91-1.82 (m, 1H), 1.47-1.33 (m, 1H), 1.32-1.21 (m, 2H), 1.09-1.01 (m, 1H), 1.00-0.93 (m, 3H), 0.76-0.68 (m, 1H).

HNMR for Example 586: ¹HNMR (400 MHz, DMSO-d₆) δ 10.92 (s, 1H), 9.40 (s, 1H), 9.39 (s, 1H), 8.72 (s, 1H), 8.34 (d, J=1.6 Hz, 1H), 8.30-8.29 (m, 1H), 7.79-7.67 (m, 2H), 3.84-3.76 (m, 4H), 3.21-3.13 (m, 4H), 3.05 (d, J=4.4 Hz, 3H), 1.91-1.82 (m, 1H), 1.47-1.33 (m, 1H), 1.32-1.21 (m, 2H), 1.09-1.01 (m, 1H), 1.00-0.93 (m, 3H), 0.76-0.68 (m, 1H).

### Example 587 and 588: Synthesis of (R)—N-(8-(methylamino)-5-(6-morpholino-[1,2,4]triazolo[1,5-a]pyridin-2-yl)-2,7-naphthyridin-3-yl)spiro[2.2]pentane-1-carboxamide formate (Example 587) and (S)—N-(8-(methylamino)-5-(6-morpholino-[1,2,4]triazolo[1,5-a]pyridin-2-yl)-2,7-naphthyridin-3-yl)spiro[2.2]pentane-1-carboxamide formate (Example 588)

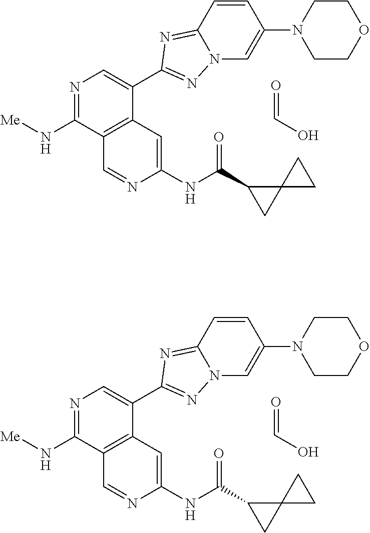

N-(8-(methylamino)-5-(6-morpholino-[1,2,4]triazolo[1,5-a]pyridin-2-yl)-2,7-naphthyridin-3-yl)spiro[2.2]pentane-1-carboxamide (Example 531) was separated by Prep-Chiral-HPLC (Column: Lux Sum Cellulose-2 2.12×25 cm, 5 μm; Mobile Phase A: Hex(0.5% 2M NH₃-MeOH), Mobile Phase B: MeOH:EtOH===1:1; Flow rate: 20 mL/min; Gradient: 70% B to 70% B in 19 min; Wave Length: 220/254 nm; R_{T1}(min): 12.74; R_{T2}(min): 17.49) to afford (R)—N-(8-(methylamino)-5-(6-morpholino-[1,2,4]triazolo[1,5-a]pyridin-2-yl)-2,7-naphthyridin-3-yl)spiro[2.2]pentane-1-carboxamide formate (Example 587, the faster peak) and (S)—N-(8-(methylamino)-5-(6-morpholino-[1,2,4]triazolo[1,5-a]pyridin-2-yl)-2,7-naphthyridin-3-yl)spiro[2.2]pentane-1-carboxamide formate (Example 588, the slower peak) as yellow solids. The two configurations are arbitrarily assigned. LCMS (ESI) m/z 471.2, [M+H]⁺.

HNMR for Example 587: ¹H NMR (400 MHz, DMSO-d₆) δ 10.72 (s, 0.1H), 9.39 (s, 0.1H), 9.36 (s, 1H), 8.70 (s, 1H), 8.36 (d, J=2.4 Hz, 1H), 8.32 (s, 1H), 8.28-8.21 (m, 1H), 7.77 (d, J=9.6 Hz, 1H), 7.69 (dd, J=9.6, 2.4 Hz, 1H), 3.83-3.76 (m, 4H), 3.20-3.13 (m, 4H), 3.05 (d, J=4.4 Hz, 3H), 2.45-2.39 (m, 1H), 1.48-1.40 (m, 1H), 1.38-1.30 (m, 1H), 0.94-0.82 (m, 3H), 0.80-0.73 (m, 1H).

HNMR for Example 588: ¹H NMR (400 MHz, DMSO-d₆) δ 10.72 (s, 1H), 9.39 (s, 1H), 9.36 (s, 1H), 8.70 (s, 1H), 8.36 (d, J=2.4 Hz, 1H), 8.32 (s, 1H), 8.28-8.21 (m, 1H), 7.77 (d, J=9.6 Hz, 1H), 7.69 (dd, J=9.6, 2.4 Hz, 1H), 3.83-3.76 (m, 4H), 3.20-3.13 (m, 4H), 3.05 (d, J=4.4 Hz, 3H), 2.45-2.39 (m, 1H), 1.48-1.40 (m, 1H), 1.38-1.30 (m, 1H), 0.94-0.82 (m, 3H), 0.80-0.73 (m, 1H).

### Example 589: Synthesis of 2-(2-fluoro-6-08-(methylamino)-5-(6-morpholino-[1,2,4]triazolo[1,5-a]pyridin-2-yl)-2,7-naphthyridin-3-yl)amino)pyridin-3-yl)propan-2-ol

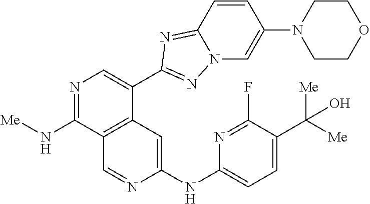

A suspension of N¹-methyl-4-(6-morpholino-[1,2,4]triazolo[1,5-c]pyridin-2-yl)-2,7-naphthyridine-1,6-diamine (Example 525, Step 1) (60.0 mg; 0.16 mmol, 1.00 eq), 2-(6-bromo-2-fluoropyridin-3-yl)propan-2-ol (37.3 mg; 0.16 mmol; 1.00 eq), EPhos Pd G₄ (14.6 mg; 0.016 mmol; 0.10 eq), EPhos (8.5 mg; 0.016 mmol; 0.10 eq) and Cs₂CO₃ (208.2 mg; 0.64 mmol; 4.00 eq) in 1,4-dioxane (5 mL) was stirred at 120° C. overnight under nitrogen atmosphere. Upon completion, the resulting mixture was concentrated under reduced pressure. The residue was purified by flash chromatography on silica gel column using 0-15% of MeOH in CH₂Cl₂ as eluent to afford 2-(2-fluoro-6-((8-(methylamino)-5-(6-morpholino-[1,2,4]triazolo[1,5-a]pyridin-2-yl)-2,7-naphthyridin-3-yl)amino)pyridin-3-yl)propan-2-ol as an off-white solid (36.7 mg, 42.7%). LCMS (ESI) m/z 530.2, [M+H]⁺. ¹H NMR (400 MHz, DMSO-d₆) δ 10.17 (s, 1H), 9.87 (s, 1H), 9.34 (s, 1H), 8.84 (s, 1H), 8.36 (s, 1H), 8.21-8.12 (m, 1H), 8.03-7.95 (m, 1H), 7.77-7.69 (m, 2H), 7.27-7.19 (m, 1H), 5.28 (s, 1H), 3.86-3.78 (m, 4H), 3.21-3.14 (m, 4H), 3.04 (d, J=4.4 Hz, 3H), 1.51 (s, 6H).

### Example 590: Synthesis of (1R,2S)-2-methyl-N-(8-(methylamino)-5-(6-((S)-2-methylmorpholino)-[1,2,4]triazolo[1,5-a]pyridin-2-yl)-2,7-naphthyridin-3-yl)cyclopropane-11-carboxamide

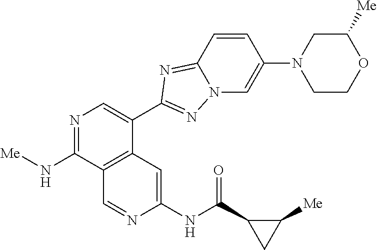

To a stirred solution of (S) N¹-methyl-4-(6-(2-methylmorpholino)-[1,2,4]triazolo[1,5-a]pyridin-2-yl)-2,7-naphthyridine-1,6-diamine (55.0 mg; 0.14 mmol; 1.00 eq.) and (1R,2S)-2-methylcyclopropane-1-carboxylic acid (12.7 mg; 0.13 mmol; 0.90 eq.) in pyridine (3 mL) was added POCl₃ (65.0 mg; 0.42 mmol; 3.01 eq.) dropwise at 0° C. The resulting solution was stirred at room temperature for 0.5 hour under nitrogen atmosphere. The resulting mixture was quenched with water (5 mL) and extracted with EtOAc (3×20 mL). The combined organic layers were washed with brine (3×1.0 mL), dried over anhydrous Na₂SO₄, filtered, and concentrated under reduced pressure. The residue was purified by flash chromatography, on silica gel column, using 0-10% of MeOH in CH₂Cl₂ as eluent to provide a crude product. The crude product was purified by flash chromatography on pre-packed C18 column using 20-100% of MeOH in water (10 mmol/L NH₄HCO₃) as eluent to provide (1R,2S)-2-methyl-5(8-(methylamino)-5-(6-((5)-2-methylmorpholino)-[1,2,4]triazolo[1,5-a]pyridin-2-yl)-2,7-naphthyridin-3-yl)cyclopropane-1-carboxamide as a yellow solid (26.0 mg, 39.0%). LCMS (ESI) m/z 473.2, [M+H]⁺. ¹H NMR (400 MHz, Methanol-d₄) δ 9.58 (s, 1H), 9.26 (s, 1H), 8.75 (s, 1H), 8.44 (d, J=1.2 Hz, 1H), 7.71-7.61 (m, 2H), 4.08-4.00 (m, 1H), 3.88-3.77 (m, 2H), 3.60-3.54 (m, 1H), 3.51-3.44 (m, 1H), 3.15 (s, 3H), 2.90-2.80 (m, 1H), 2.54-2.45 (m, 1H), 2.08-2.00 (m, 1H), 1.49-1.39 (m, 1H), 1.27 (d, J=6.4 Hz, 3H), 1.22 (d, J=6.4 Hz, 3H), 1.11-0.99 (m, 2H).

### Example 591: Synthesis of N-(5-((4-methoxyphenyl)ethynyl)-8-(methylamino)-2,7-naphthyridin-3-yl)cyclopropanecarboxamide

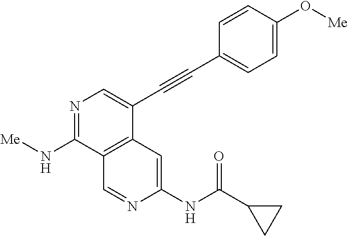 [SMILES: CNc1ncc(C#Cc2ccc(OC)cc2)c2cc(NC(=O)C3CC3)ncc12]

To a solution of N-(5-bromo-8-(methylamino)-2,7-naphthyridin-3-yl)cyclopropanecarboxamide (500.0 mg; 1.557 mmol; 1.00 eq.) and 1-ethynyl-4-methoxybenzene (266.0 mg; 2.013 mmol; 1.29 eq.) in DMF (15 mL) were added XPhos (74.0 mg; 0.155 mmol; 0.10 eq.), Et₃N (627.0 mg; 6.196 mmol; 3.98 eq.), XPhos Pd G₃ (131.0 mg; 0.155 mmol; 0.10 eq.) and CuI (30.0 mg; 0.158 mmol; 0.10 eq.). The resulting solution was stirred at 90° C. for 1 hour under nitrogen atmosphere. The desired product was detected via LCMS. The resulting mixture was diluted with EtOAc (50 mL) and washed with brine (3×10 mL). The organic layer was dried over anhydrous Na₂SO₄. After filtration, the filtrate was concentrated under reduced pressure. The residue was purified by flash chromatography on silica gel column using 1-9% of MeOH in CH₂Cl₂ as eluent to provide N-(5-((4-methoxyphenyl)ethynyl)-8-(methylamino)-2,7-naphthyridin-3-yl)cyclopropanecarboxamide as a yellow solid (303.6 mg, 52.3%). LCMS (ESI) m/z 373.2, [M+H]⁺. ¹H NMR (400 MHz, DMSO-d₆) δ 11.05 (s, 1H), 9.37 (s, 1H), 8.64 (s, 1H), 8.30-8.25 (m, 1H), 8.24 (s, 1H), 7.50 (d, J=8.8 Hz, 2H), 7.02 (d, J=8.8 Hz, 2H), 3.81 (s, 3H), 3.02 (d, J=4.4 Hz, 3H), 2.15-2.03 (m, 1H), 0.94-0.80 (m, 4H).

### Examples 592 and 593

Each compound in Table 25 below was prepared using a similar experimental procedure to prepare example 591, using N-(5-bromo-8-(methylamino)-2,7-naphthyridin-3-yl)cyclopropanecarboxamide as the common intermediate and appropriate alkyne.

TABLE 25

| Example | Structure and Name | MS and ¹H NMR |
|---|---|---|
| Example 592 | 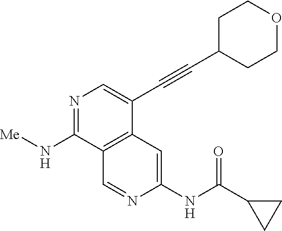 [SMILES: CNc1ncc(C#CC2CCOCC2)c2cc(NC(=O)C3CC3)ncc12] | LCMS (ESI) m/z 351.2, [M + H]⁺. |
| | N-(8-(methylamino)-5- | |
| | ((tetrahydro-2H-pyran-4- | |
| | yl)ethynyl)-2,7-naphthyridin- | |
| | 3-yl)cyclopropanecarboxamide | |
| Example 593 | 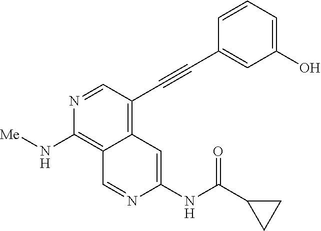 [SMILES: CNc1ncc(C#Cc2cccc(O)c2)c2cc(NC(=O)C3CC3)ncc12] | LCMS (ESI) m/z 359.1, [M + H]⁺. ¹H NMR (400 MHz, DMSO-d₆) δ 11.04 (s, 1H), 9.65 (s, 1H), 9.37 (s, 1H), 8.62 (s, 1H), 8.33-8.25 (m, 2H), 7.27-7.19 (m, 1H), 7.00 (d, J = 7.6 Hz, 1H), 6.90 (d, J = 1.6 Hz, 1H), 6.84-6.77 (m, 1H), 3.02 (d, J = 4.4 Hz, 3H), 2.14-2.04 (m, 1H), 0.95-0.80 (m, 4H). |
| | N-(5-((3- | |
| | hydroxyphenyl)ethynyl)-8- | |
| | (methylamino)-2,7- | |
| | naphthyridin-3- | |
| | yl)cyclopropanecarboxamide | |

### Example 594: Synthesis of N-(8-(ethylamino)-5-((5-methoxypyridin-2-yl)ethynyl)-2,7-naphthyridin-3-yl)cyclopropanecarboxamide

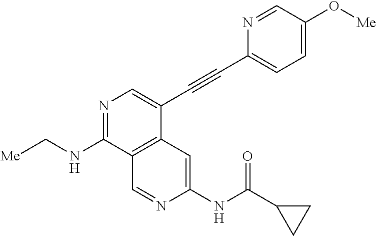 [SMILES: CCNc1ncc(C#Cc2ccc(OC)cn2)c2cc(NC(=O)C3CC3)ncc12]

### Step 1: 6-chloro-N-ethyl-2,7-naphthyridin-1-amine

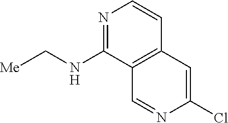 [SMILES: CCNc1nccc2cc(Cl)ncc12]

To a solution of 6-chloro-2,7-naphthyridin-1(2H)-one (12.50 g; 69.210 mmol; 1.00 eq.) and PyBOP (73.01 g; 140.271 mmol; 2.03 eq.) in DMA (150 mL) was added DIEA (46.01 g; 355.901 mmol; 5.14 eq.) at room temperature. The resulting solution was stirred at 40° C. for 3 hours under nitrogen atmosphere. To the above solution was added ethanamine in EtOH (30% w/w) (53.00 g; 352.665 mmol; 5.10 eq.). The reaction was stirred at 40° C. for another 16 hours under nitrogen atmosphere. The desired product was detected via LCMS. The resulting mixture was diluted with water (500 mL) and extracted with CH₂Cl₂ (2×200 mL). The organic layers were washed with water (2×200 mL) and dried over anhydrous Na₂SO₄. After filtration, the filtrate was concentrated under reduced pressure to precipitate solids. The precipitated solids were collected by filtration and washed with CH₂Cl₂ (20 mL) to afford 6-chloro-N-ethyl-2,7-naphthyridin-1-amine as a off-white solid (10.01 g, 69.5%). LCMS (ESI) m/z 208.1, [M+H]⁺.

### Step 2: N-(8-(ethylamino)-2,7-naphthyridin-3-yl)cyclopropanecarboxamide

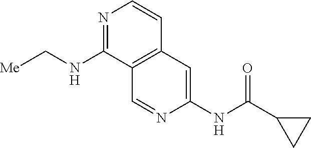 [SMILES: CCNc1nccc2cc(NC(=O)C3CC3)ncc12]

To a solution of 6-chloro-N-ethyl-2,7-naphthyridin-1-amine (9.40 g; 45.260 mmol; 1.00 eq.) and cyclopropanecarboxamide (5.8 g; 68.150 mmol; 1.51 eq.) in 1,4-dioxane (150 mL) was added XantPhos (5.20 g; 8.980 mmol; 0.20 eq.), Cs₂CO₃ (30.00 g; 92.071 mmol; 2.03 eq.) and Pd₂(dba)₃ (4.30 g; 4.691 mmol; 0.10 eq.) at room temperature under nitrogen atmosphere. The resulting mixture was stirred at 110° C. for 16 hours. The desired product was detected via LCMS. The mixture was allowed to cool down to room temperature. The resulting mixture was concentrated under reduced pressure and purified by flash chromatography on silica gel column using 2-8% of MeOH in CH₂Cl₂ as eluent to afford N-(8-(ethylamino)-2,7-naphthyridin-3-yl)cyclopropanecarboxamide as a white solid (10.00 g, 86.1%).LCMS (ESI) m/z 257.1, [M+H]⁺.

### Step 3: N-(5-bromo-8-(ethylamino)-2,7-naphthyridin-3-yl)cyclopropanecarboxamide

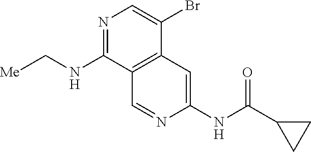 [SMILES: CCNc1ncc(Br)c2cc(NC(=O)C3CC3)ncc12]

To a solution of N-(8-(ethylamino)-2,7-naphthyridin-3-yl)cyclopropanecarboxamide (9.00 g; 35.190 mmol; 1.00 eq.) in DMF (30 mL) was added NBS (5.22 g; 29.350 mmol; 0.80 eq.) in portions. The resulting solution was stirred at room temperature for 1 hour under nitrogen atmosphere. The desired product was detected via LCMS. The resulting mixture was diluted with water (500 mL), whereupon the precipitated solids were collected via filtration, washed with water (100 mL) and dried under reduced pressure to afford N-(5-bromo-8-(ethylamino)-2,7-naphthyridin-3-yl)cyclopropanecarboxamide as an off-white solid (1.0.02 g, 99.5%). LCMS (ESI) m/z 335.0, [M+H]⁺.

### Step 4: N-(8-(ethylamino)-5-((5-methoxypyridin-2-yl)ethynyl)-2,7-naphthyridin-3-yl)cyclopropanecarboxamide

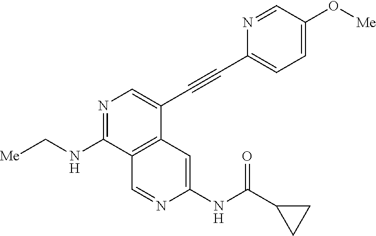 [SMILES: CCNc1ncc(C#Cc2ccc(OC)cn2)c2cc(NC(=O)C3CC3)ncc12]

N-(8-(ethylamino)-5-((5-methoxypyridin-2-yl)ethynyl)-2,7-naphthyridin-3-yl)cyclopropanecarboxamide was synthesized using a similar procedure that was previously described in Example 591, by using N-(5-bromo-8-(ethylamino)-2,7-naphthyridin-3-yl)cyclopropanecarboxamide and 2-ethynyl-5-methoxypyridine as the starting material. LCMS (ESI) m/z 388.2, [M+H]⁺. ¹H NMR (300 MHz, DMSO-d₆) δ 11.08 (s, 1H), 9.43 (s, 1H), 8.62 (s, 1H), 8.36-8.25 (m, 3H), 7.60 (d, J=8.7 Hz, 1H), 7.50-7.44 (m, 1H), 3.89 (s, 3H), 3.66-3.51 (m, 2H), 2.15-2.02 (m, 1H), 1.26 (t, J=6.9 Hz, 3H), 0.94-0.79 (m, 4H).

### Example 595: Synthesis of N-(5-((1-methyl-1H-benzo[d][1,2,3]triazol-6-yl)ethynyl)-8-(methylamino)-2,7-naphthyridin-3-yl)cyclopropanecarboxamide

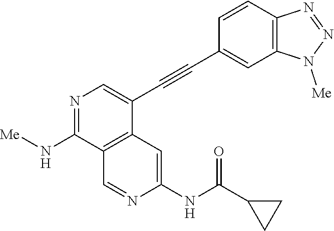 [SMILES: CNc1ncc(C#Cc2ccc3nnn(C)c3c2)c2cc(NC(=O)C3CC3)ncc12]

### Step 1: N-(8-(methylimino)-5-((trimethylsilyl)ethynyl)-2,7-naphthyridin-3-yl)cyclopropane carboxamide

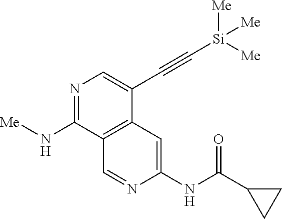 [SMILES: CNc1ncc(C#C[Si](C)(C)C)c2cc(NC(=O)C3CC3)ncc12]

To a stirred solution of N-(5-bromo-8-(methylamino)-2,7-naphthyridin-3-yl)cyclopropanecarboxamide (5.00 g; 15.570 mmol; 1.00 eq.) in DMF (50 mL) were added Pd(dppf)Cl₂·CH₂Cl₂ (6.34 g; 7.780 mmol; 0.50 eq.), CuI (1.48 g; 7.770 mmol; 0.50 eq.), DIEA. (12.07 g; 93.390 mmol; 6.00 eq.) and ethynyltrimethylsilane (3.06 g; 31.160 mmol; 2.00 eq.). The resulting solution was stirred at 50° C. overnight. The desired product was detected via LCMS. The mixture was purified by flash chromatography on pre-packed C18 column using 20-1.00% of MeCN in water (5 mmol/L NH₄HCO₃) as eluent to provide N-(8-(methylamino)-5-((trimethylsilyl)ethynyl)-2,7-naphthyridin-3-yl)cyclopropanecarboxamide (5.0 g, 95.0%) as a yellow solid. LCMS (ESI) m/z 339.2, [M+H]⁺.

### Step 2: N-(5-ethynyl-8-(methylamino)-2,7-naphthyridin-3-yl)cyclopropanecarboxamide

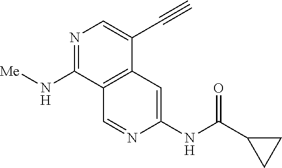 [SMILES: C#Cc1cnc(NC)c2cnc(NC(=O)C3CC3)cc12]

To a mixture of N-(8-(methylamino)-5-((trimethylsilyl)ethynyl)-2,7-naphthyridin-3-yl)cyclopropanecarboxamide (5.00 g; 14.770 mmol; 1.00 eq.) in MeOH (100 mL) was added K₂CO₃ (5.46 g; 39.510 mmol; 2.67 eq.). The resulting solution was stirred at room temperature for 2 h under nitrogen atmosphere. The mixture was concentrated under vacuum. The residue was purified by flash chromatography on silica gel column using 0-20% of MeOH in CH₂Cl₂ as eluent to provide N-(5-ethynyl-8-(methylamino)-2,7-naphthyridin-3-yl)cyclopropanecarboxamide (3.0 g, 76.3%) as a white solid. LCMS (ESI) m/z 267.1, [M+H]⁺.

### Step 3: N-(5-((1-methyl-1H-benzo[d][1,2,3]triazol-6-yl)ethynyl)-8-(methylamino)-2,7-naphthyridin-3-yl)cyclopropanecarboxamide

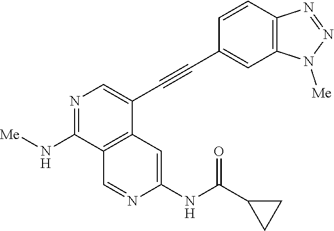 [SMILES: CNc1ncc(C#Cc2ccc3nnn(C)c3c2)c2cc(NC(=O)C3CC3)ncc12]

To a solution of 6-bromo-1-methyl-1H-benzo[d][1,2,3]triazole (160.0 mg; 0.755 mmol; 0.67 eq.), CuI (47.1 mg; 0.247 mmol; 0.22 eq.), XPhos (120.0 mg; 0.252 mmol; 0.22 eq.) and XPhos Pd G₃ (200.0 mg; 0.236 mmol; 0.21 eq.) in DMF (8 mL) were added N-(5-ethynyl-8-(methylamino)-2,7-naphthyridin-3-yl)cyclopropanecarboxamide (300.0 mg; 1.1.27 mmol; 1.00 eq.) and Et₃N (240.0 mg; 2.372 mmol; 2.11 eq.) under nitrogen atmosphere. The resulting solution was stirred at 90° C. for 1 hour. The reaction was diluted with EtOAc (80 mL) and washed by brine (2×30 mL). The organic layer was dried over anhydrous Na₂SO₄. After filtration, the filtrate was concentrated under reduced pressure. The residue was purified by flash chromatography on silica gel column using 5-10% of MeOH in CH₂Cl₂ as eluent to provide N-(5-((1-methyl-1H-benzo[d][1,2,3]triazol-6-yl)ethynyl)-8-(methylamino)-2,7-naphthyridin-3-yl)cyclopropanecarboxamide as a yellow solid (86.2 mg, 20.1%). LCMS (ESI) m/z 398.2, [M+H]⁺. ¹H NMR (400 MHz, DMSO-d₆) δ 11.14 (s, 1H), 9.39 (s, 1H), 8.77 (s, 1H), 8.43-8.27 (m, 1H), 8.32 (s, 1H), 8.1.7 (s, 1H), 8.09 (d, J=8.4 Hz, 1H), 7.53 (dd, J=8.4, 1.0 Hz, 1H), 4.34 (s, 3H), 3.03 (d, J=4.4 Hz, 3H), 2.13-2.08 (m, 1H), 0.94-0.82 (m, 41-1).

### Examples 596-601 and 606-676

Each compound in Table 26 below was prepared using a similar experimental procedure to prepare Example 595, using N-(5-ethynyl-8-(methylamino)-2,7-naphthyridin-3-yl)cyclopropanecarboxamide as the common intermediate and appropriate aryl halide.

TABLE 26

| Example | Structure and Name | MS and ¹H NMR |
|---|---|---|
| Example 596 | 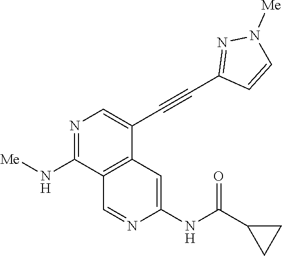 [SMILES: CNc1ncc(C#Cc2ccn(C)n2)c2cc(NC(=O)C3CC3)ncc12] N-(5-((1-methyl-1H-pyrazol-3- yl)ethynyl)-8-(methylamino)-2,7- naphthyridin-3- yl)cyclopropanecarboxamide | LCMS (ESI) m/z 347.0, [M + H]⁺. ¹H NMR (400 MHz, Methanol-d₄) δ 9.21 (s, 1H), 8.65 (s, 1H), 8.21 (s, 1H), 7.64 (d, J = 2.4 Hz, 1H), 6.59 (d, J = 2.4 Hz, 1H), 3.94 (s, 3H), 3.11 (s, 3H), 2.02-1.91 (m, 1H), 1.09-1.01 (m, 2H), 0.97- 0.88 (m, 2H). |
| Example 597 | 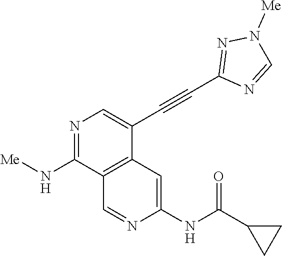 [SMILES: CNc1ncc(C#Cc2ncn(C)n2)c2cc(NC(=O)C3CC3)ncc12] N-(5-((1-methyl-1H-1,2,4-triazol-3- yl)ethynyl)-8-(methylamino)-2,7- naphthyridin-3- yl)cyclopropanecarboxamide | LCMS (ESI) m/z 348.1, [M + H]⁺. ¹H NMR (400 MHz, DMSO-d₆) δ 11.09 (s, 1H), 9.38 (s, 1H), 8.57 (s, 1H), 8.53 (s, 1H), 8.45-8.41 (m, 1 H), 8.34 (s, 1H), 3.92 (s, 3H), 3.03 (d, J = 4.4 Hz, 3H), 2.12-2.02 (m, 1H), 0.92-0.79 (m, 4H). |
| Example 598 | 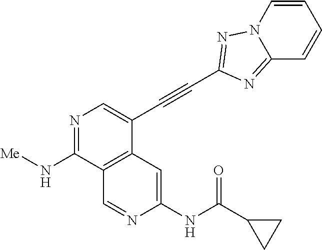 [SMILES: CNc1ncc(C#Cc2nc3ccccn3n2)c2cc(NC(=O)C3CC3)ncc12] N-(5-([1,2,4]triazolo[1,5-a]pyridin-2- ylethynyl)-8-(methylamino)-2,7- naphthyridin-3- yl)cyclopropanecarboxamide | LCMS (ES) m/z 384.1, [M + H]⁺. ¹H NMR (400 MHz, DMSO-d₆) δ 11.13 (s, 1H), 9.43 (s, 1H), 8.97 (d, J = 6.8 Hz, 1H), 8.64-8.59 (m, 1H), 8.59 (s, 1H), 8.42 (s, 1H), 7.87 (d, J = 8.4 Hz, 1H), 7.79- 7.70 (m, 1H), 7.31-7.26 (m, 1H), 3.06 (d, J = 4.4 Hz, 3H), 2.14- 2.04 (m, 1H), 0.90-0.83 (m, 4H). |
| Example 599 | 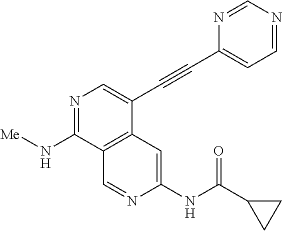 [SMILES: CNc1ncc(C#Cc2ccncn2)c2cc(NC(=O)C3CC3)ncc12] N-(8-(methylamino)-5-(pyrimidin-4- ylethynyl)-2,7-naphthyridin-3- yl)cyclopropanecarboxamide | LCMS (ESI) m/z 345.1, [M + H]⁺. ¹H NMR (400 MHz, DMSO-d₆) δ 11.13 (s, 1H), 9.40 (s, 1H), 9.19 (d, J = 1.2 Hz, 1H), 8.85 (d, J = 5.2 Hz, 1H), 8.63 (s, 1H), 8.62- 8.58 (m, 1H), 8.44 (s, 1H), 7.65 (dd, J = 5.2, 1.2 Hz , 1H), 3.05 (d, J = 4.4 Hz, 3H), 2.13-2.07 (m, 1H), 0.98-0.82 (m, 4H). |
| Example 600 | 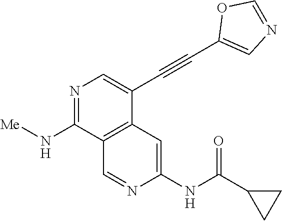 [SMILES: CNc1ncc(C#Cc2cnco2)c2cc(NC(=O)C3CC3)ncc12] N-(8-(methylamino)-5-(oxazol-5- ylethynyl)-2,7-naphthyridin-3- yl)cyclopropanecarboxamide | LCMS (ESI) m/z 334.1, [M + H]⁺. ¹H NMR (400 MHz, DMSO-d₆) δ 11.10 (s, 1H), 9.39 (s, 1H), 8.57- 8.45 (m, 3H), 8.35 (s, 1H), 7.60 (s, 1H), 3.04 (d, J = 4.4 Hz, 3H), 2.15- 2.05 (m, 1H), 0.93-0.86 (m, 4H). |
| Example 601 | 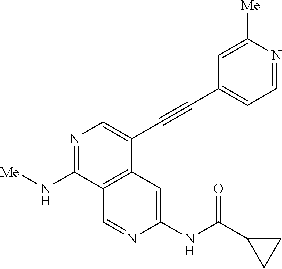 [SMILES: CNc1ncc(C#Cc2ccnc(C)c2)c2cc(NC(=O)C3CC3)ncc12] N-(8-(methylamino)-5-((2- methylpyridin-4-yl)ethynyl)-2,7- naphthyridin-3- yl)cyclopropanecarboxamide | LCMS (ESI) m/z 358.2, [M + H]⁺. ¹H NMR (400 MHz, DMSO-d₆) δ 11.12 (s, 1H), 9.39 (s, 1H), 8.69 (s, 1H), 8.55-8.40 (m, 2H), 8.33 (s, 1H), 7.44 (s, 1H), 7.28 (d, J = 5.2 Hz, 1H), 3.04 (d, J = 4.4 Hz, 3H), 2.50 (s, 3H), 2.13-2.07 (m, 1H), 0.92-0.86 (m, 4H). |
| Example 606 | 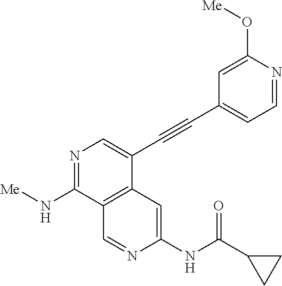 [SMILES: CNc1ncc(C#Cc2ccnc(OC)c2)c2cc(NC(=O)C3CC3)ncc12] N-(5-((2-methoxypyridin-4- yl)ethynyl)-8-(methylamino)-2,7- naphthyridin-3- yl)cyclopropanecarboxamide | LCMS (ESI) m/z 374.2, [M + H]⁺. ¹H NMR (400 MHz, DMSO-d₆) δ 11.12 (s, 1H), 9.39 (s, 1H), 8.66 (s, 1H), 8.49-8.42 (m, 1H), 8.34 (s, 1H), 8.22 (d, J = 5.2 Hz, 1H), 7.09 (dd, J = 5.2, 1.2 Hz, 1H), 6.93 (d, J = 1.2 Hz, 1H), 3.89 (s, 3H), 3.03 (d, J = 4.4 Hz, 3H), 2.13-2.06 (m, 1H), 0.91-0.84 (m, 4H). |
| Example 607 | 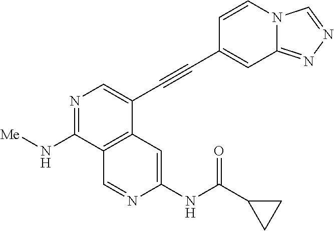 [SMILES: CNc1ncc(C#Cc2ccn3cnnc3c2)c2cc(NC(=O)C3CC3)ncc12] N-(5-([1,2,4]triazolo[4,3-a]pyridin-7- ylethynyl)-8-(methylamino)-2,7- naphthyridin-3- yl)cyclopropanecarboxamide | LCMS (ESI) m/z 384.1, [M + H]⁺. ¹H NMR (400 MHz, DMSO-d₆) δ 11.14 (s, 1H), 9.40 (s, 1H), 9.28 (s, 1H), 8.70 (s, 1H), 8.62 (d, J = 7.2 Hz, 1H), 8.50-8.43 (m, 1H), 8.36 (s, 1H), 7.92 (d, J = 1.2 Hz, 1H), 7.05 (dd, J = 7.2, 1.2 Hz, 1H), 3.04 (d, J = 4.4 Hz, 3H), 2.16-2.05 (m, 1H), 0.96-0.90 (m, 2H), 0.90- 0.83 (m, 2H). |
| Example 608 | 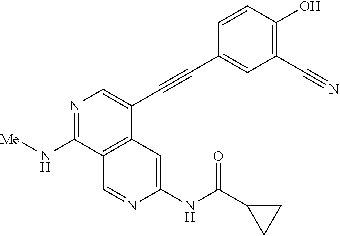 [SMILES: CNc1ncc(C#Cc2ccc(O)c(C#N)c2)c2cc(NC(=O)C3CC3)ncc12] N-(5-((3-cyano-4- hydroxyphenyl)ethynyl)-8- (methylamino)-2,7-naphthyridin-3- yl)cyclopropanecarboxamide | LCMS (ESI) m/z 384.1, [M + H]⁺. ¹H NMR (400 MHz, DMSO-d₆) δ 11.62 (s, 1H), 11.10 (s, 1H), 9.37 (s, 1H), 8.64 (s, 1H), 8.37-8.31 (m, 1H), 8.23 (s, 1H), 7.84 (d, J = 2.0 Hz, 1H), 7.64 (dd, J = 8.4, 2.0 Hz, 1H), 7.07 (d, J = 8.4 Hz, 1H), 3.04 (d, J = 4.4 Hz, 3H), 2.10- 2.04 (m, 1H), 0.92-0.81 (m, 4H). |
| Example 609 | 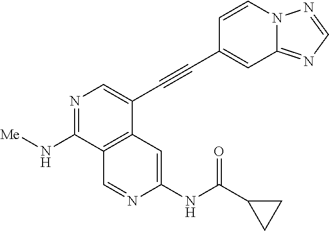 [SMILES: CNc1ncc(C#Cc2ccn3ncnc3c2)c2cc(NC(=O)C3CC3)ncc12] N-(5-([1,2,4]triazolo[1,5-a]pyridin-7- ylethynyl)-8-(methylamino)-2,7- naphthyridin-3- yl)cyclopropanecarboxamide | LCMS (ESI) m/z 384.1, [M + H]⁺. ¹H NMR (400 MHz, DMSO-d₆) δ 11.13 (s, 1H), 9.40 (s, 1H), 9.02 (d, J = 7.2 Hz, 1H), 8.71 (s, 1H), 8.57 (s, 1H), 8.51-8.49 (m, 1H), 8.37 (s, 1H), 7.98 (s, 1H), 7.27 (d, J = 7.2 Hz, 1H), 3.05 (d, J = 4.4 Hz, 3H), 2.11 (m, 1H), 0.97-0.83 (m, 4H). |
| Example 610 | 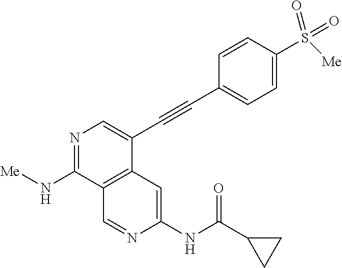 [SMILES: CNc1ncc(C#Cc2ccc(S(C)(=O)=O)cc2)c2cc(NC(=O)C3CC3)ncc12] N-(8-(methylamino)-5-((4- (methylsulfonyl)phenyl)ethynyl)-2,7- naphthyridin-3- yl)cyclopropanecarboxamide | LCMS (ESI) m/z 421.1 [M + H]⁺. ¹H NMR (400 MHz, DMSO-d₆) δ 11.14 (s, 1H), 9.41 (s, 1H), 8.69 (s, 1H), 8.69-8.43 (m, 1H), 8.34 (s, 1H), 7.99 (d, J = 8.4 Hz, 2H), 7.80 (d, J = 8.4 Hz, 2H), 3.27 (s, 3H), 3.05 (d, J = 4.4 Hz, 3H), 2.13- 2.07 (m, 1H), 0.94-0.93 (m, 2H), 0.92-0.91 (m, 2H). |
| Example 611 | 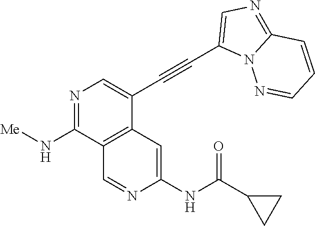 [SMILES: CNc1ncc(C#Cc2cnc3cccnn23)c2cc(NC(=O)C3CC3)ncc12] N-(5-(imidazo[1,2-b]pyridazin-3- ylethynyl)-8-(methylamino)-2,7- naphthyridin-3- yl)cyclopropanecarboxamide | LCMS (ESI) m/z 384.1, [M + H]⁺. ¹H NMR (400 MHz, DMSO-d₆) δ 11.06 (s, 1H), 9.40 (s, 1H), 8.71- 8.65 (m, 2H), 8.47-8.38 (m, 1H), 8.36 (s, 1H), 8.28-8.21 (m, 1H), 8.16 (s, 1H), 7.37 (dd, J = 9.2, 4.4 Hz, 1H), 3.05 (d, J = 4.4 Hz, 3H), 2.14-2.04 (m, 1H), 0.89-0.76 (m, 4H). |
| Example 612 | 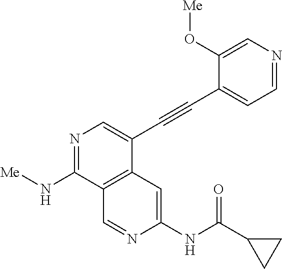 [SMILES: CNc1ncc(C#Cc2ccncc2OC)c2cc(NC(=O)C3CC3)ncc12] N-(5-((3-methoxypyridin-4- yl)ethynyl)-8-(methylamino)-2,7- naphthyridin-3- yl)cyclopropanecarboxamide | LCMS (ESI) m/z 374.2 [M + H]⁺. ¹H NMR (400 MHz, DMSO-d₆) δ 11.09 (s, 1H), 9.39 (s, 1H), 8.67 (s, 1H), 8.49-8.42 (m, 2H), 8.31 (s, 1H), 8.23 (d, J = 4.8 Hz, 1H), 7.44 (d, J = 4.8 Hz, 1H), 4.03 (s, 3H), 3.04 (d, J = 4.4 Hz, 3H), 2.11- 2.08 (m, 1H), 0.91-0.86 (m, 4H). |
| Example 613 | 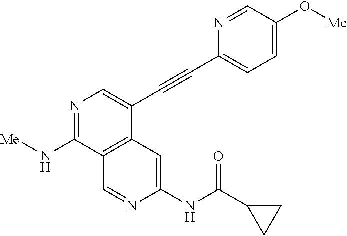 [SMILES: CNc1ncc(C#Cc2ccc(OC)cn2)c2cc(NC(=O)C3CC3)ncc12] N-(5-((5-methoxypyridin-2- ylethynyl)-8-(methylamino)-2,7- naphthyridin-3- yl)cyclopropanecarboxamide | LCMS (ESI) m/z 374.2, [M + H]⁺. ¹H NMR (400 MHz, DMSO-d₆) δ 11.08 (s, 1H), 9.38 (s, 1H), 8.62 (s, 1H), 8.40-8.29 (m, 3H), 7.60 (d, J = 8.6 Hz, 1H), 7.47 (dd, J = 8.6, 3.0 Hz, 1H), 3.88 (s, 3H), 3.03 (d, J = 4.4 Hz, 3H), 2.09-2.04 (m, 1H), 0.89-0.82 (m, 4H). |
| Example 614 | 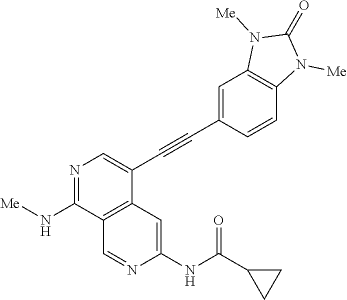 [SMILES: CNc1ncc(C#Cc2ccc3c(c2)n(C)c(=O)n3C)c2cc(NC(=O)C3CC3)ncc12] N-(5-((1,3-dimethyl-2-oxo-2,3- dihydro-1H-benzo[d]imidazol-5- yl)ethynyl)-8-(methylamino)-2,7- naphthyridin-3- yl)cyclopropanecarboxamide | LCMS (ESI) m/z 427.2, [M + H]⁺. ¹H NMR (400 MHz, DMSO-d₆) δ 11.10 (s, 1H), 9.38 (s, 1H), 8.76 (s, 1H), 8.31-8.26 (m, 1H), 8.24 (s, 1H), 7.46 (d, J = 1.5 Hz, 1H), 7.30 (dd, J = 8.0, 1.5 Hz, 1H), 7.22 (d, J = 8.0 Hz, 1H), 3.38 (s, 3H), 3.36 (s, 3H), 3.02 (d, J = 4.4 Hz, 3H), 2.15-2.05 (m, 1H), 0.94-0.81 (m, 4H). |
| Example 615 | 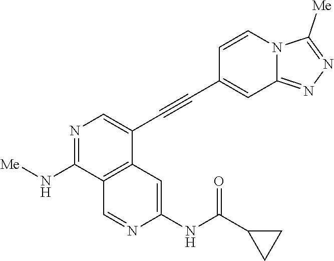 [SMILES: CNc1ncc(C#Cc2ccn3c(C)nnc3c2)c2cc(NC(=O)C3CC3)ncc12] N-(5-((3-methyl-[1,2,4]triazolo[4,3- a]pyridin-7-yl)ethynyl)-8- (methylamino)-2,7-naphthyridin-3- yl)cyclopropanecarboxamide | LCMS (ESI) m/z 398.0, [M + H]⁺. ¹H NMR (400 MHz, DMSO-d₆) δ 11.13 (s, 1H), 9.39 (s, 1H), 8.71 (s, 1H), 8.55-8.42 (m, 2H), 8.35 (s, 1H), 7.84 (s, 1H), 7.04 (d, J = 7.2 Hz, 1H), 3.04 (d, J = 4.4 Hz, 3H), 2.71 (s, 3H), 2.13-2.08 (m, 1H), 0.94-0.85 (m, 4H). |
| Example 616 | 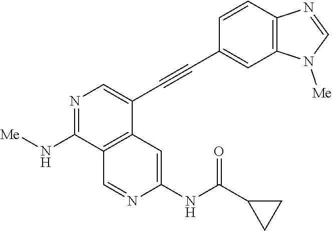 [SMILES: CNc1ncc(C#Cc2ccc3ncn(C)c3c2)c2cc(NC(=O)C3CC3)ncc12] N-(5-((1-methyl-1H- benzo[d]imidazol-6-yl)ethynyl)-8- (methylamino)-2,7-naphthyridin-3- yl)cyclopropanecarboxamide | LCMS (ESI) m/z 397.2 [M + H]⁺. ¹H NMR (400 MHz, DMSO-d₆) δ 11.12 (s, 1H), 9.39 (s, 1H), 8.79 (s, 1H), 8.32-8.28 (m, 3H), 7.92 (s, 1H), 7.69 (d, J = 8.4 Hz, 1H), 7.42- 7.37 (m, 1H), 3.89 (s, 3H), 3.03 (d, J = 4.4 Hz, 3H), 2.12-2.08 (m, 1H), 0.92-0.85 (m, 4H). |
| Example 617 | 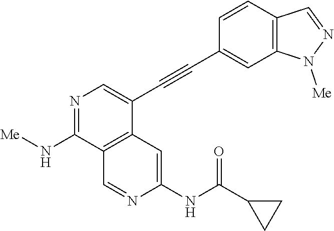 [SMILES: CNc1ncc(C#Cc2ccc3cnn(C)c3c2)c2cc(NC(=O)C3CC3)ncc12] N-(5-((1-methyl-1H-indazol-6- ylethynyl)-8-(methylamino)-2,7- naphthyridin-3- yl)cyclopropanecarboxamide | LCMS (ESI) m/z 397.2, [M + H]⁺. ¹H NMR (400 MHz, DMSO-d₆) δ 11.12 (s, 1H), 9.39 (s, 1H), 8.80 (s, 1H), 8.43-8.33 (m, 1H), 8.30 (s, 1H), 8.09 (s, 1H), 7.99 (s, 1H), 7.81 (d, J = 8.4 Hz, 1H), 7.34- 7.25 (m, 1H), 4.10 (s, 3H), 3.03 (d, J = 4.4 Hz, 3H), 2.17-2.06 (m, 1H), 0.98-0.77 (m, 4H). |
| Example 618 | 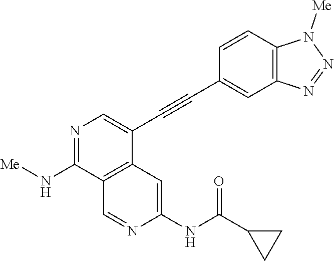 [SMILES: CNc1ncc(C#Cc2ccc3c(c2)nnn3C)c2cc(NC(=O)C3CC3)ncc12] N-(5-((1-methyl-1H- benzo[d][1,2,3]triazol-5-yl)ethynyl)-8- (methylamino)-2,7-naphthyridin-3- yl)cyclopropanecarboxamide | LCMS (ESI) m/z 398.0, ¹H NMR (400 MHz, DMSO-d₆) δ 11.10 (s, 1H), 9.39 (s, 1H), 8.74 (s, 1H), 8.37-8.34 (m, 1H), 8.32 (s, 1H), 8.21 (s, 1H), 7.96 (d, J = 8.6 Hz, 1H), 7.73 (d, J = 8.6 Hz, 1H), 4.35 (s, 3H), 3.04 (d, J = 4.4 Hz, 3H), 2.16-2.05 (m, 1H), 0.96-0.81 (m, 4H). |
| Example 619 | 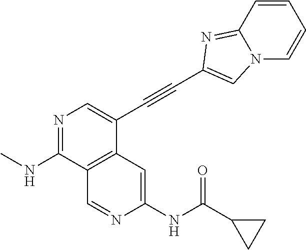 [SMILES: CNc1ncc(C#Cc2cn3ccccc3n2)c2cc(NC(=O)C3CC3)ncc12] N-(5-(imidazo[1,2-a]pyridin-2- ylethynyl)-8-(methylamino)-2,7- naphthyridin-3- yl)cyclopropanecarboxamide | LCMS (ESI) m/z 383.2, [M + H]⁺. ¹H NMR (400 MHz, DMSO-d₆) δ 11.07 (s, 1H), 9.38 (s, 1H), 8.59 (s, 1H), 8.57-8.51 (m, 1H), 8.40- 8.35 (m, 1H), 8.32 (s, 1H), 8.25 (s, 1H), 7.60-7.51 (m, 1H), 7.37- 7.27 (m, 1H), 6.99-6.91 (m, 1H), 3.03 (d, J = 4.4 Hz, 3H), 2.14- 2.03 (m, 1H), 0.93-0.81 (m, 4H). |
| Example 620 | 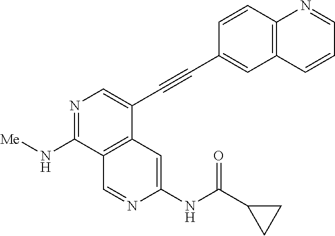 [SMILES: CNc1ncc(C#Cc2ccc3ncccc3c2)c2cc(NC(=O)C3CC3)ncc12] N-(8-(methylamino)-5-(quinolin-6- ylethynyl)-2,7-naphthyridin-3- yl)cyclopropanecarboxamide | LCMS (ESI) m/z 394.2, [M + H]⁺. ¹H NMR (400 MHz, DMSO-d₆) δ 11.12 (s, 1H), 9.40 (s, 1H), 8.93 (dd, J = 4.4, 1.6, 1H), 8.74 (s, 1H), 8.42-8.38 (m, 2H), 8.35 (s, 1H), 8.25 (d, J = 1.6 Hz, 1H), 8.07 (d, J = 8.8 Hz, 1H), 7.87 (dd, J = 8.8, 1.6 Hz, 1H), 7.61 (dd, J = 8.4, 4.4 Hz, 1H), 3.04 (d, J = 4.4 Hz, 3H), 2.14-2.08 (m, 1H), 0.98-0.81 (m, 4H). |
| Example 621 | 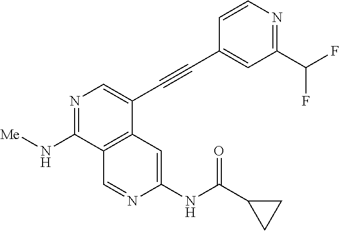 [SMILES: CNc1ncc(C#Cc2ccnc(C(F)F)c2)c2cc(NC(=O)C3CC3)ncc12] N-(5-((2-(difluoromethyl)pyridin-4- yl)ethynyl)-8-(methylamino)-2,7- naphthyridin-3- yl)cyclopropanecarboxamide | LCMS (ESI) m/z 394.1, [M + H]⁺. ¹H NMR (400 MHz, DMSO-d₆) δ 11.14 (s, 1H), 9.39 (s, 1H), 8.80- 8.69 (m, 2H), 8.60-8.51 (m, 1H), 8.38 (s, 1H), 7.83 (s, 1H) 7.64 (d, J = 4.8 Hz, 1H), 6.98 (t, J = 54.8 Hz, 1H), 3.04 (d, J = 4.4 Hz, 3H), 2.13-2.06 (m, 1H), 0.95-0.88 (m, 4H). |
| Example 622 | 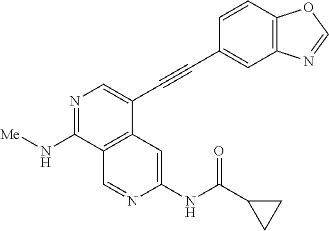 [SMILES: CNc1ncc(C#Cc2ccc3ocnc3c2)c2cc(NC(=O)C3CC3)ncc12] N-(5-(benzo[d]oxazol-5-ylethynyl)-8- (methylamino)-2,7-naphthyridin-3- yl)cyclopropanecarboxamide | LCMS (ESI) m/z 384.1, [M + H]⁺. ¹H NMR (400 MHz, DMSO-d₆) δ 11.10 (s, 1H), 9.38 (s, 1H), 8.85 (s, 1H), 8.71 (s, 1H), 8.38-8.35 (m, 1H), 8.31 (s, 1H), 7.99 (s, 1H), 7.87 (d, J = 8.4 Hz, 1H), 7.64 (d, J = 8.4 Hz, 1H), 3.03 (d, J = 4.4 Hz, 3H), 2.14-2.05 (m, 1H), 0.94- 0.88 (m, 2H), 0.88-0.80 (m, 2H). |
| Example 623 | 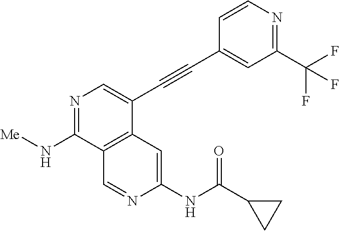 [SMILES: CNc1ncc(C#Cc2ccnc(C(F)(F)F)c2)c2cc(NC(=O)C3CC3)ncc12] N-(8-(methylamino)-5-((2- (trifluoromethyl)pyridin-4- yl)ethynyl)-2,7-naphthyridin-3- yl)cyclopropanecarboxamide | LCMS (ESI) m/z 412.1, [M + H]⁺. ¹H NMR (400 MHz, DMSO-d₆) δ 11.16 (s, 1H), 9.39 (s, 1H), 8.81 (d, J = 4.8 Hz, 1H), 8.77 (s, 1H), 8.59-8.54 (m, 1H), 8.39 (s, 1H), 8.06 (s, 1H), 7.77 (d, J = 4.8 Hz, 1H), 3.04 (d, J = 4.4 Hz, 3H), 2.13- 2.08 (m, 1H), 0.98-0.82 (m, 4H). |
| Example 624 | 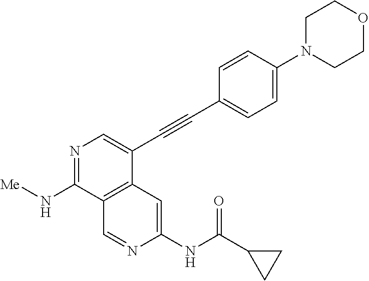 [SMILES: CNc1ncc(C#Cc2ccc(N3CCOCC3)cc2)c2cc(NC(=O)C3CC3)ncc12] N-(8-(methylamino)-5-((4- morpholinophenyl)ethynyl)-2,7- naphthyridin-3- yl)cyclopropanecarboxamide | LCMS (ESI) m/z 428.1, [M + H]⁺. ¹H NMR (400 MHz, DMSO-d₆) δ 11.06 (s, 1H), 9.36 (s, 1H), 8.65 (s, 1H), 8.27-8.23 (m, 1H), 8.22 (s, 1H), 7.42 (d, J = 8.8 Hz, 2H), 6.99 (d, J = 8.8 Hz, 2H), 3.78-3.71 (m, 4H), 3.23-3.15 (m, 4H), 3.01 (d, J = 4.4 Hz, 3H), 2.14-2.03 (m, 1H), 0.94-0.80 (m, 4H). |
| Example 625 | 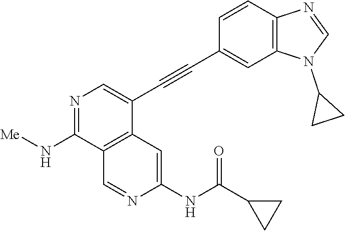 [SMILES: CNc1ncc(C#Cc2ccc3ncn(C4CC4)c3c2)c2cc(NC(=O)C3CC3)ncc12] N-(5-((1-cyclopropyl-1H- benzo[d]imidazol-6-yl)ethynyl)-8- (methylamino)-2,7-naphthyridin-3- yl)cyclopropanecarboxamide | LCMS (ESI) m/z 423.2, [M + H]⁺. ¹H NMR (400 MHz, DMSO-d₆) δ 11.11 (s, 1H), 9.39 (s, 1H), 8.77 (s, 1H), 8.35-8.29 (m, 3H), 7.89 (s, 1H), 7.69 (d, J = 8.4 Hz, 1H), 7.41 (dd, J = 8.4, 1.6 Hz, 1H), 3.61- 3.52 (m, 1H), 3.03 (d, J = 4.4 Hz, 3H), 2.14-2.06 (m, 1H), 1.19- 1.05 (m, 4H), 0.92-0.81 (m, 4H). |
| Example 626 | 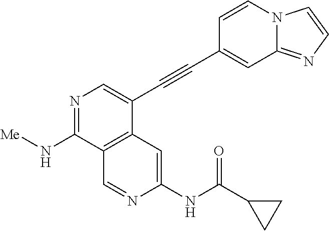 [SMILES: CNc1ncc(C#Cc2ccn3ccnc3c2)c2cc(NC(=O)C3CC3)ncc12] N-(5-(imidazo[1,2-a]pyridin-7- ylethynyl)-8-(methylamino)-2,7- naphthyridin-3- yl)cyclopropanecarboxamide | LCMS (ESI) m/z 383.2, [M + H]⁺. ¹H NMR (400 MHz, DMSO-d₆) δ 11.10 (s, 1H), 9.39 (s, 1H), 8.70 (s, 1H), 8.61 (dd, J = 7.0, 1.0 Hz, 1H), 8.43-8.36 (m, 1H), 8.34 (s, 1H), 8.00 (s, 1H), 7.77-7.64 (m, 2H), 7.00 (dd, J = 7.0, 1.5 Hz, 1H), 3.04 (d, J = 4.4 Hz, 3H), 2.16- 2.03 (m, 1H), 1.02-0.74 (m, 4H). |
| Example 627 | 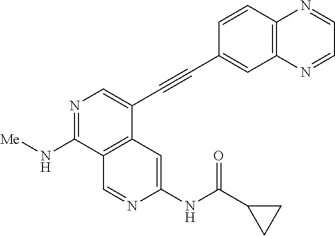 [SMILES: CNc1ncc(C#Cc2ccc3nccnc3c2)c2cc(NC(=O)C3CC3)ncc12] N-(8-(methylamino)-5-(quinoxalin-6- ylethynyl)-2,7-naphthyridin-3- yl)cyclopropanecarboxamide | LCMS (ESI) m/z 395.2, [M + H]⁺. ¹H NMR (400 MHz, DMSO-d₆) δ 11.12 (s, 1H), 9.40 (s, 1H), 9.01 (d, J = 1.6 Hz, 1H), 8.96 (d, J = 1.6 Hz, 1H), 8.75 (s, 1H), 8.48-8.42 (m, 1H), 8.39 (s, 1H), 8.24 (s, 1H), 8.15 (d, J = 8.4 Hz, 1H), 7.98 (dd, J = 8.8, 1.6 Hz, 1H), 3.05 (d, J = 4.4 Hz, 3H), 2.17-2.08 (m, 1H), 0.99-0.82 (m, 4H). |
| Example 628 | 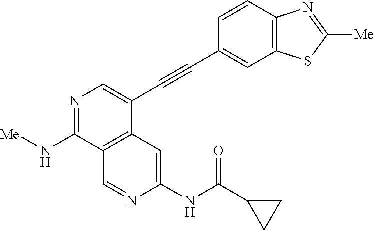 [SMILES: CNc1ncc(C#Cc2ccc3nc(C)sc3c2)c2cc(NC(=O)C3CC3)ncc12] N-(8-(methylamino)-5-((2- methylbenzo[d]thiazol-6-yl)ethynyl)- 2,7-naphthyridin-3- yl)cyclopropanecarboxamide | LCMS (ESI) m/z 414.1, [M + H]⁺. ¹H NMR (400 MHz, DMSO-d₆) δ 11.11 (s, 1H), 9.38 (s, 1H), 8.69 (s, 1H), 8.39-8.35 (m, 1H), 8.30 (s, 1H), 8.28 (d, J = 1.6 Hz, 1H), 7.95 (d, J = 8.4 Hz, 1H), 7.64 (dd, J = 8.4, 1.6 Hz, 1H), 3.03 (d, J = 4.4 Hz, 3H), 2.83 (s, 3H), 2.14-2.06 (m, 1H), 0.94-0.82 (m, 4H). |
| Example 629 | 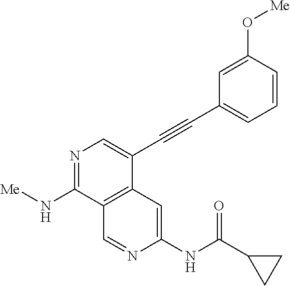 [SMILES: CNc1ncc(C#Cc2cccc(OC)c2)c2cc(NC(=O)C3CC3)ncc12] N-(5-((3-methoxyphenyl)ethynyl)-8- (methylamino)-2,7-naphthyridin-3- yl)cyclopropanecarboxamide | LCMS (ESI) m/z 373.2, [M + H]⁺. ¹H NMR (400 MHz, DMSO-d₆) δ 11.11 (s, 1H), 9.38 (s, 1H), 8.74 (s, 1H), 8.39-8.36 (m, 1H), 8.28 (s, 1H), 7.40-7.32 (m, 1H), 7.24- 7.11 (m, 2H), 7.02-6.93 (m, 1H), 3.84 (s, 3H), 3.03 (d, J = 4.4 Hz, 3H), 2.15-2.03 (m, 1H), 0.95- 0.78 (m, 4H). |
| Example 630 | 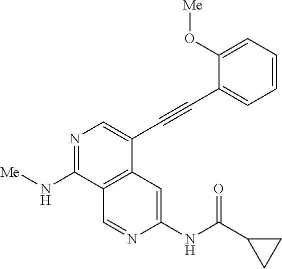 [SMILES: CNc1ncc(C#Cc2ccccc2OC)c2cc(NC(=O)C3CC3)ncc12] N-(5-((2-methoxyphenyl)ethynyl)-8- (methylamino)-2,7-naphthyridin-3- yl)cyclopropanecarboxamide | LCMS (ESI) m/z 373.2, [M + H]⁺. ¹H NMR (400 MHz, DMSO-d₆) δ 11.03 (s, 1H), 9.37 (s, 1H), 8.66 (s, 1H), 8.31-8.27 (m, 1H), 8.25 (s, 1H), 7.53-7.50 (m, 1H), 7.40- 7.34 (m, 1H), 7.12-7.08 (m, 1H), 7.02-6.96 (m, 1H), 3.90 (s, 3H), 3.02 (d, J = 4.4 Hz, 3H), 2.12- 2.06 (m, 1H), 0.92-0.83 (m, 4H). |
| Example 631 | 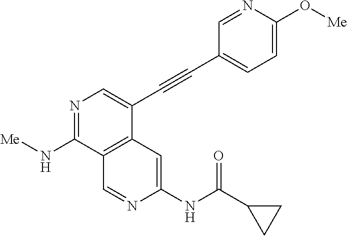 [SMILES: CNc1ncc(C#Cc2ccc(OC)nc2)c2cc(NC(=O)C3CC3)ncc12] N-(5-((6-methoxypyridin-3- yl)ethynyl)-8-(methylamino)-2,7- naphthyridin-3- yl)cyclopropanecarboxamide | LCMS (ESI) m/z 374.2, [M + H]⁺. ¹H NMR (400 MHz, DMSO-d₆) δ 11.08 (s, 1H), 9.37 (s, 1H), 8.64 (s, 1H), 8.40 (d, J = 2.0 Hz, 1H), 8.37- 8.30 (m, 1H), 8.28 (s, 1H), 7.86 (dd, J = 8.4, 2.4 Hz, 1H), 6.93 (d, J = 8.4 Hz, 1H), 3.91 (s, 3H), 3.02 (d, J = 4.4 Hz, 3H), 2.11-2.06 (m, 1H), 0.92-0.81 (m, 4H). |
| Example 632 | 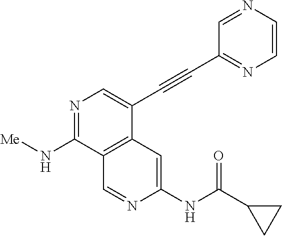 [SMILES: CNc1ncc(C#Cc2cnccn2)c2cc(NC(=O)C3CC3)ncc12] N-(8-(methylamino)-5-(pyrazin-2- ylethynyl)-2,7-naphthyridin-3- yl)cyclopropanecarboxamide | LCMS (ESI) m/z 345.1, [M + H]⁺. ¹H NMR (400 MHz, DMSO-d₆) δ 11.11 (s, 1H), 9.40 (s, 1H), 8.85 (s, 1H), 8.69 (d, J = 2.0 Hz, 1H), 8.65 (s, 1H), 8.60 (d, J = 2.0 Hz, 1H), 8.65-8.59 (m, 1H), 8.41 (s, 1H), 3.05 (d, J = 4.4 Hz, 3H), 2.15- 2.06 (m, 1H), 0.94-0.82 (m, 4H). |
| Example 633 | 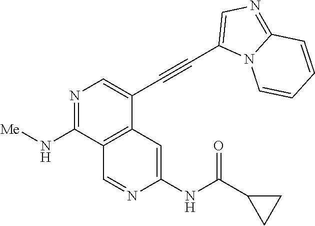 [SMILES: CNc1ncc(C#Cc2cnc3ccccn23)c2cc(NC(=O)C3CC3)ncc12] N-(5-(imidazo[1,2-a]pyridin-3- ylethynyl)-8-(methylamino)-2,7- naphthyridin-3- yl)cyclopropanecarboxamide | LCMS (ESI) m/z 383.2, [M + H]⁺. ¹H NMR (400 MHz, DMSO-d₆) δ 11.18 (s, 1H), 9.41 (s, 1H), 8.76 (d, J = 6.4 Hz, 1H), 8.71 (s, 1H), 8.41-8.39 (m, 1H), 8.38 (s, 1H), 8.00 (s, 1H), 7.74 (d, J = 8.8 Hz, 1H), 7.51-7.42 (m, 1H), 7.16- 7.10 (m, 1H), 3.05 (d, J = 4.4 Hz, 3H), 2.14-2.08 (m, 1H), 0.98- 0.83 (m, 4H). |
| Example 634 | 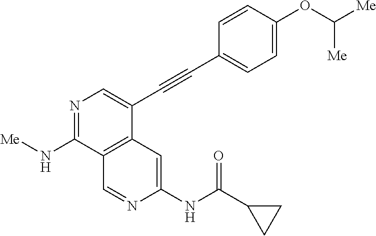 [SMILES: CNc1ncc(C#Cc2ccc(OC(C)C)cc2)c2cc(NC(=O)C3CC3)ncc12] N-(5-((4-isopropoxyphenyl)ethynyl)- 8-(methylamino)-2,7-naphthyridin-3- yl)cyclopropanecarboxamide | LCMS (ESI) m/z 401.2, [M + H]⁺. ¹H NMR (400 MHz, DMSO-d₆) δ 11.05 (s, 1H), 9.37 (s, 1H), 8.63 (s, 1H), 8.35-8.26 (m, 1H), 8.24 (s, 1H), 7.47 (d, J = 8.8 Hz, 2H), 6.98 (d, J = 8.8 Hz, 2H), 4.72-4.64 (m, 1H), 3.02 (d, J = 4.4 Hz, 3H), 2.11- 2.07 (m, 1H), 1.31 (d, J = 6.8 Hz, 6H), 0.92-0.81 (m, 4H). |
| Example 635 | 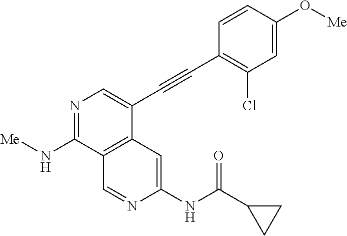 [SMILES: CNc1ncc(C#Cc2ccc(OC)cc2Cl)c2cc(NC(=O)C3CC3)ncc12] N-(5-((2-chloro-4- methoxyphenyl)ethynyl)-8- (methylamino)-2,7-naphthyridin-3- yl)cyclopropanecarboxamide | LCMS (ESI) m/z 407.0, [M + H]⁺. ¹H NMR (400 MHz, DMSO-d₆) δ 11.03 (s, 1H), 9.38 (s, 1H), 8.64 (s, 1H), 8.38-8.30 (m, 1H), 8.27 (s, 1H), 7.58 (d, J = 8.8 Hz, 1H), 7.20 (d, J = 2.8 Hz, 1H), 7.09-6.88 (m, 1H), 3.83 (s, 3H), 3.03 (d, J = 4.4 Hz, 3H), 2.15-1.98 (m, 1H), 0.93- 0.80 (m, 4H). |
| Example 636 | 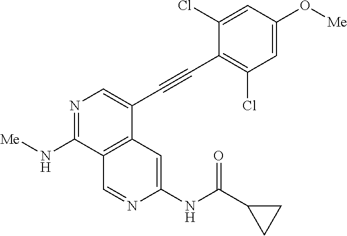 [SMILES: CNc1ncc(C#Cc2c(Cl)cc(OC)cc2Cl)c2cc(NC(=O)C3CC3)ncc12] N-(5-((2,6-dichloro-4- methoxyphenyl)ethynyl)-8- (methylamino)-2,7-naphthyridin-3- yl)cyclopropanecarboxamide | LCMS (ESI) m/z 441.1, [M + H]⁺. ¹H NMR (400 MHz, DMSO-d₆) δ 11.03 (s, 1H), 9.39 (s, 1H), 8.65 (s, 1H), 8.49-8.38 (m, 1H), 8.28 (s, 1H), 7.24 (s, 2H), 3.84 (s, 3H), 3.03 (d, J = 4.4 Hz, 3H), 2.12- 2.01 (m, 1H), 0.89-0.80 (m, 4H). |
| Example 637 | 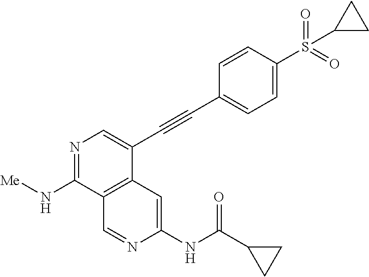 [SMILES: CNc1ncc(C#Cc2ccc(S(=O)(=O)C3CC3)cc2)c2cc(NC(=O)C3CC3)ncc12] N-(5-((4- (cyclopropylsulfonyl)phenyl)ethynyl)- 8-(methylamino)-2,7-naphthyridin-3- yl)cyclopropanecarboxamide | LCMS (ESI) m/z 447.1, [M + H]⁺. ¹H NMR (400 MHz, DMSO-d₆) δ 11.11 (s, 1H), 9.39 (s, 1H), 8.68 (s, 1H), 8.48-8.41 (m, 1H), 8.35 (s, 1H), 7.95 (d, J = 8.4 Hz, 2H), 7.79 (d, J = 8.4 Hz, 2H), 3.04 (d, J = 4.4 Hz, 3H), 2.96-2.87 (m, 1H) 2.13-2.05 (m, 1H), 1.20-1.04 (m, 4H), 0.95-0.81 (m, 4H). |
| Example 638 | 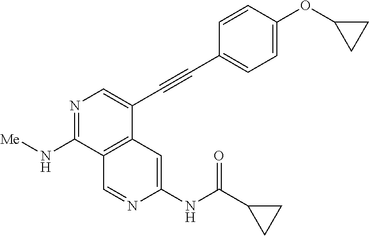 [SMILES: CNc1ncc(C#Cc2ccc(OC3CC3)cc2)c2cc(NC(=O)C3CC3)ncc12] N-(5-((4- cyclopropoxyphenyl)ethynyl)-8- (methylamino)-2,7-naphthyridin-3- yl)cyclopropanecarboxamide | LCMS (ESI) m/z 399.2, [M + H]⁺. ¹H NMR (400 MHz, DMSO-d₆) δ 11.05 (s, 1H), 9.37 (s, 1H), 8.64 (s, 1H), 8.29-8.26 (m, 1H), 8.25 (s, 1H), 7.50 (dd, J = 8.8, 2.4 Hz, 2H), 7.12 (dd, J = 8.8, 2.4 Hz, 2H), 3.94-3.89 (m, 1H), 3.02 (d, J = 4.4 Hz, 3H), 2.11-2.06 (m, 1H), 0.92-0.79 (m, 6H), 0.72- 0.68 (m, 2H). |
| Example 639 | 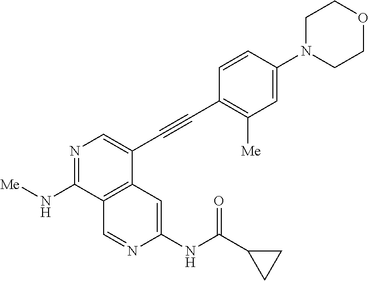 [SMILES: CNc1ncc(C#Cc2ccc(N3CCOCC3)cc2C)c2cc(NC(=O)C3CC3)ncc12] N-(5-((2-methyl-4- morpholinophenyl)ethynyl)-8- (methylamino)-2,7-naphthyridin-3- yl)cyclopropanecarboxamide | LCMS (ESI) m/z 442.2, [M + H]⁺. ¹H NMR (400 MHz, DMSO-d₆) δ 11.08 (s, 1H), 9.38 (s, 1H), 8.67 (s, 1H), 8.38-8.25 (m, 1H), 8.20 (s, 1H), 7.36 (d, J = 8.8 Hz, 1H), 6.91 (d, J = 2.0 Hz, 1H), 6.82 (dd, J = 8.4, 2.0 Hz, 1H), 3.78-3.71 (m, 4H), 3.22-3.15 (m, 4H), 3.03 (d, J = 4.4 Hz, 3H), 2.48 (s, 3H), 2.13- 2.02 (m, 1H), 0.91-0.79 (m, 4H). |
| Example 640 | 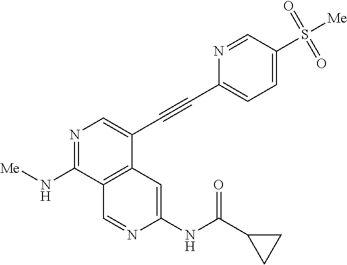 [SMILES: CNc1ncc(C#Cc2ccc(S(C)(=O)=O)cn2)c2cc(NC(=O)C3CC3)ncc12] N-(8-(methylamino)-5-((5- (methylsulfonyl)pyridin-2- yl)ethynyl)-2,7-naphthyridin-3- yl)cyclopropanecarboxamide | LCMS (ESI) m/z 422.1, [M + H]⁺. ¹H NMR (400 MHz, DMSO-d₆) δ 11.13 (s, 1H), 9.40 (s, 1H), 9.06 (d, J = 2.4 Hz, 1H), 8.65 (s, 1H), 8.59-8.51 (m, 1H), 8.43 (s, 1H), 8.40-8.34 (m, 1H), 7.85 (d, J = 8.4 Hz , 1H), 3.31 (s, 3H), 3.05 (d, J = 4.4 Hz, 3H), 2.18-1.98 (m, 1H), 0.99-0.79 (m, 4H). |
| Example 641 | 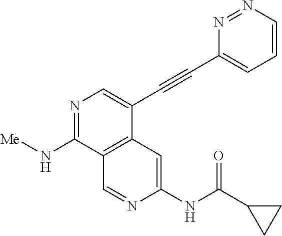 [SMILES: CNc1ncc(C#Cc2cccnn2)c2cc(NC(=O)C3CC3)ncc12] N-(8-(methylamino)-5-(pyridazin-3- ylethynyl)-2,7-naphthyridin-3- yl)cyclopropanecarboxamide | LCMS (ESI) m/z 345.2, [M + H]⁺. ¹H NMR (400 MHz, DMSO-d₆) δ 11.13 (s, 1H), 9.41 (s, 1H), 9.21 (dd, J = 5.2, 1.6 Hz, 1H), 8.65 (s, 1H), 8.58-8.50 (m, 1H), 8.43 (s, 1H), 7.88 (dd, J = 8.4, 1.6 Hz, 1H), 7.79 (dd, J = 8.4, 5.2 Hz, 1H), 3.05 (d, J = 4.4 Hz, 3H), 2.12- 2.06 (m, 1H), 0.93-0.82 (m, 4H). |
| Example 642 | 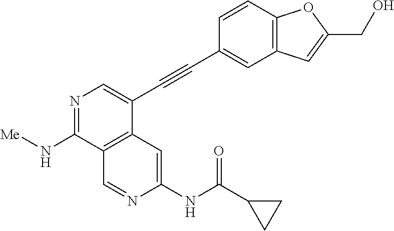 [SMILES: CNc1ncc(C#Cc2ccc3oc(CO)cc3c2)c2cc(NC(=O)C3CC3)ncc12] N-(5-((2-(hydroxymethyl)benzofuran- 5-yl)ethynyl)-8-(methylamino)-2,7- naphthyridin-3- yl)cyclopropanecarboxamide | LCMS (ESI) m/z 413.2, [M + H]⁺. ¹H NMR (400 MHz, DMSO-d₆) δ 11.07 (s, 1H), 9.37 (s, 1H), 8.68 (s, 1H), 8.30-8.28 (m, 2H), 7.83 (d, J = 1.8 Hz, 1H), 7.62 (d, J = 8.4 Hz, 1H), 7.47 (dd, J = 8.4, 1.8 Hz, 1H), 6.79 (s, 1H), 5.53 (t, J = 6.0 Hz, 1H), 4.60 (d, J = 5.9 Hz, 2H), 3.03 (d, J = 4.4 Hz, 3H), 2.10- 2.06 (m, 1H), 0.95-0.80 (m, 4H). |
| Example 643 | 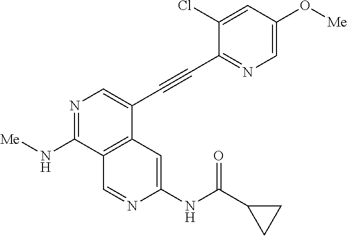 [SMILES: CNc1ncc(C#Cc2ncc(OC)cc2Cl)c2cc(NC(=O)C3CC3)ncc12] N-(5-((3-chloro-5-methoxypyridin-2- yl)ethynyl)-8-(methylamino)-2,7- naphthyridin-3- yl)cyclopropanecarboxamide | LCMS (ESI) m/z 408.1, [M + H]⁺. ¹H NMR (400 MHz, DMSO-d₆) δ 11.05 (s, 1H), 9.39 (s, 1H), 8.63 (s, 1H), 8.48-8.39 (m, 1H), 8.34 (s, 1H), 8.29 (d, J = 2.8 Hz, 1H), 7.74 (d, J = 2.8 Hz, 1H), 3.91 (s, 3H), 3.04 (d, J = 4.4 Hz, 3H), 2.12- 2.03 (m, 1H), 0.91-0.80 (m, 4H). |
| Example 644 | 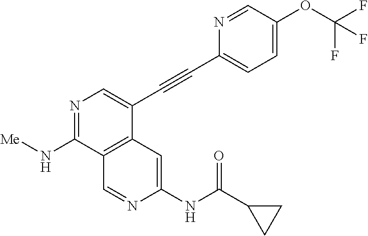 [SMILES: CNc1ncc(C#Cc2ccc(OC(F)(F)F)cn2)c2cc(NC(=O)C3CC3)ncc12] N-(8-(methylamino)-5-((5- (trifluoromethoxy)pyridin-2- yl)ethynyl)-2,7-naphthyridin-3- yl)cyclopropanecarboxamide | LCMS (ESI) m/z 428.1, [M + H]⁺. ¹H NMR (400 MHz, DMSO-d₆) δ 11.12 (s, 1H), 9.39 (s, 1H), 8.70 (d, J = 2.8 Hz, 1H), 8.62 (s, 1H), 8.52-8.47 (m, 1H), 8.38 (s, 1H), 8.01 (dd, J = 8.8, 2.4 Hz, 1H), 7.76 (d, J = 8.8 Hz, 1H), 3.04 (d, J = 4.4 Hz, 3H), 2.11-2.05 (m, 1H), 0.91-0.86 (m, 4H). |
| Example 645 | 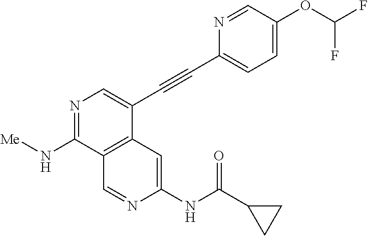 [SMILES: CNc1ncc(C#Cc2ccc(OC(F)F)cn2)c2cc(NC(=O)C3CC3)ncc12] N-(5-((5-(difluoromethoxy)pyridin-2- yl)ethynyl)-8-(methylamino)-2,7- naphthyridin-3- yl)cyclopropanecarboxamide | LCMS (ESI) m/z 410.1, [M + H]⁺. ¹H NMR (400 MHz, DMSO-d₆) δ 11.11 (s, 1H), 9.39 (s, 1H), 8.62 (s, 1H), 8.52 (d, J = 2.8 Hz, 1H), 8.45- 8.44 (m, 1H), 8.36 (s, 1H), 7.78- 7.75 (m, 1H), 7.71 (d, J = 8.4 Hz, 1H), 7.40 (t, J = 73.2 Hz, 1H), 3.04 (d, J = 4.4 Hz, 3H), 2.11- 2.05 (m, 1H), 0.91-0.86 (m, 4H). |
| Example 646 | 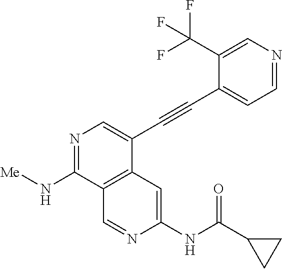 [SMILES: CNc1ncc(C#Cc2ccncc2C(F)(F)F)c2cc(NC(=O)C3CC3)ncc12] N-(8-(methylamino)-5-((3- (trifluoromethyl)pyridin-4- yl)ethynyl)-2,7-naphthyridin-3- yl)cyclopropanecarboxamide | LCMS (ESI) m/z 412.1, [M + H]⁺. ¹H NMR (400 MHz, DMSO-d₆) δ 11.10 (s, 1H), 9.40 (s, 1H), 8.99 (s, 1H), 8.90 (d, J = 4.8 Hz, 1H), 8.64- 8.60 (m, 1H), 8.57 (s, 1H), 8.34 (s, 1H), 7.73 (d, J = 4.8 Hz, 1H), 3.05 (d, J = 4.4 Hz, 3H), 2.14- 2.03 (m, 1H), 0.94-0.81 (m, 4H). |
| Example 647 | 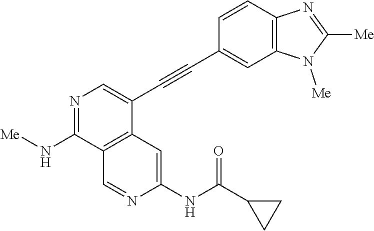 [SMILES: CNc1ncc(C#Cc2ccc3nc(C)n(C)c3c2)c2cc(NC(=O)C3CC3)ncc12] N-(5-((1,2-dimethyl-1H- benzo[d]imidazol-6-yl)ethynyl)-8- (methylamino)-2,7-naphthyridin-3- yl)cyclopropanecarboxamide | LCMS (ESI) m/z 411.2, [M + H]⁺. ¹H NMR (400 MHz, DMSO-d₆) δ 11.12 (s, 1H), 9.39 (s, 1H), 8.80 (s, 1H), 8.33-8.29 (m, 1H), 8.27 (s, 1H), 7.85 (s, 1H), 7.55 (d, J = 8.4 Hz, 1H), 7.33 (dd, J = 8.4, 1.6 Hz, 1H), 3.79 (s, 3H), 3.03 (d, J = 4.5 Hz, 3H), 2.57 (s, 3H), 2.12-2.08 (m, 1H), 0.93-0.82 (m, 4H). |
| Example 648 | 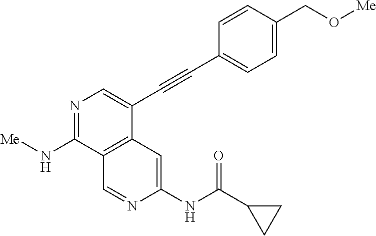 [SMILES: CNc1ncc(C#Cc2ccc(COC)cc2)c2cc(NC(=O)C3CC3)ncc12] N-(5-((4- (methoxymethyl)phenyl)ethynyl)-8- (methylamino)-2,7-naphthyridin-3- yl)cyclopropanecarboxamide | LCMS (ESI) m/z 387.1, [M + H]⁺. ¹H NMR (400 MHz, DMSO-d₆) δ 11.06 (s, 1H), 9.37 (s, 1H), 8.66 (s, 1H), 8.34-8.31 (m, 1H), 8.28 (s, 1H), 7.55 (d, J = 8.0 Hz, 2H), 7.38 (d, J = 8.0 Hz, 2H), 4.45 (s, 2H), 3.32 (s, 3H), 3.03 (d, J = 4.4 Hz, 3H), 2.11-2.05 (m, 1H), 0.92- 0.89 (m, 4H). |
| Example 649 | 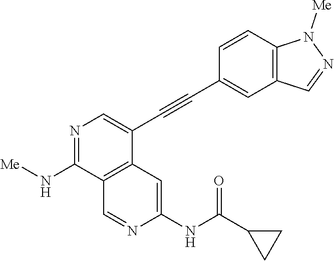 [SMILES: CNc1ncc(C#Cc2ccc3c(cnn3C)c2)c2cc(NC(=O)C3CC3)ncc12] N-(5-((1-methyl-1H-indazol-5- yl)ethynyl)-8-(methylamino)-2,7- naphthyridin-3- yl)cyclopropanecarboxamide | LCMS (ESI) m/z 397.2, [M + H]⁺. ¹H NMR (400 MHz, DMSO-d₆) δ 11.08 (s, 1H), 9.38 (s, 1H), 8.71 (s, 1H), 8.34-8.27 (m, 2H), 8.10 (s, 1H), 7.99 (s, 1H), 7.74 (d, J = 8.8 Hz, 1H), 7.57 (dd, J = 8.8, 1.4 Hz, 1H), 4.09 (s, 3H), 3.03 (d, J = 4.4 Hz, 3H), 2.15-2.03 (m, 1H), 0.96- 0.81 (m, 4H). |
| Example 650 | 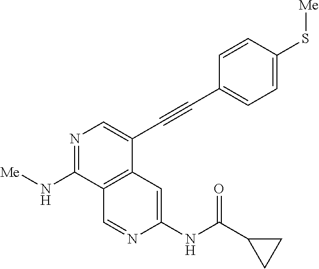 [SMILES: CNc1ncc(C#Cc2ccc(SC)cc2)c2cc(NC(=O)C3CC3)ncc12] N-(8-(methylamino)-5-((4- (methylthio)phenyl)ethynyl)-2,7- naphthyridin-3- yl)cyclopropanecarboxamide | LCMS (ESI) m/z 389.1, [M + H]⁺. ¹H NMR (400 MHz, DMSO-d₆) δ 11.08 (s, 1H), 9.37 (s, 1H), 8.65 (s, 1H), 8.35-8.30 (m, 1H), 8.27 (s, 1H), 7.54-7.44 (m, 2H), 7.37- 7.29 (m, 2H), 3.02 (d, J = 4.4 Hz, 3H), 2.55 (s, 3H), 2.15-2.04 (m, 1H), 0.97-0.81 (m, 4H). |
| Example 651 | 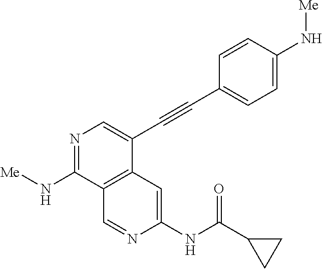 [SMILES: CNc1ccc(C#Cc2cnc(NC)c3cnc(NC(=O)C4CC4)cc23)cc1] N-(8-(methylamino)-5-((4- (methylamino)phenyl)ethynyl)-2,7- naphthyridin-3- yl)cyclopropanecarboxamide | LCMS (ESI) m/z 372.2, [M + H]⁺. ¹H NMR (400 MHz, DMSO-d₆) δ 11.04 (s, 1H), 9.35 (s, 1H), 8.63 (s, 1H), 8.27-8.16 (m, 2H), 7.35- 7.23 (m, 2H), 6.65-6.51 (m, 2H), 6.16-6.07 (m, 1H), 3.01 (d, J = 4.4 Hz, 3H), 2.72 (d, J = 5.6 Hz, 3H), 2.15-2.06 (m, 1H), 0.99- 0.82 (m, 4H). |
| Example 652 | 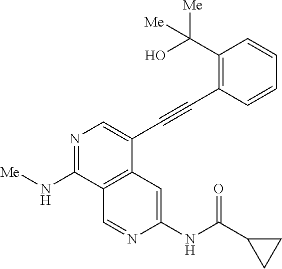 [SMILES: CNc1ncc(C#Cc2ccccc2C(C)(C)O)c2cc(NC(=O)C3CC3)ncc12] N-(5-((2-(2-hydroxypropan-2- yl)phenyl)ethynyl)-8-(methylamino)- 2,7-naphthyridin-3- yl)cyclopropanecarboxamide | LCMS (ESI) m/z 401.2, [M + H]⁺. ¹H NMR (400 MHz, DMSO-d₆) δ 11.05 (s, 1H), 9.38 (s, 1H), 8.61 (s, 1H), 8.36-8.29 (m, 1H), 8.26 (s, 1H), 7.84-7.78 (m, 1H), 7.59- 7.51 (m, 1H), 7.40-7.32 (m, 1H), 7.30-7.23 (m, 1H), 5.10 (s, 1H), 3.03 (d, J = 4.4 Hz, 3H), 2.14- 2.05 (m, 1H), 1.72 (s, 6H), 0.96- 0.78 (m, 4H). |
| Example 653 | 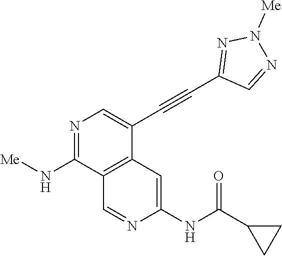 [SMILES: CNc1ncc(C#Cc2cnn(C)n2)c2cc(NC(=O)C3CC3)ncc12] N-(5-((2-methyl-2H-1,2,3-triazol-4- yl)ethynyl)-8-(methylamino)-2,7- naphthyridin-3- yl)cyclopropanecarboxamide | LCMS (ESI) m/z 348.2, [M + H]⁺. ¹H NMR (400 MHz, DMSO-d₆) δ 11.09 (s, 1H), 9.38 (s, 1H), 8.54 (s, 1H), 8.42-8.39 (m, 1H), 8.31 (s, 1H), 8.04 (s, 1H), 4.21 (s, 3H), 3.03 (d, J = 4.4 Hz, 3H), 2.10- 2.04 (m, 1H), 0.88-0.83 (m, 4H). |
| Example 654 | 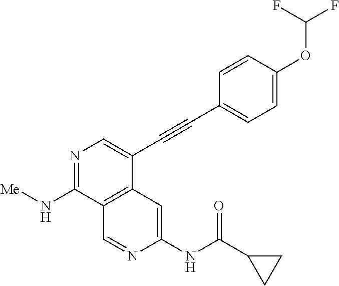 [SMILES: CNc1ncc(C#Cc2ccc(OC(F)F)cc2)c2cc(NC(=O)C3CC3)ncc12] N-(5-((4- (difluoromethoxy)phenyl)ethynyl)-8- (methylamino)-2,7-naphthyridin-3- yl)cyclopropanecarboxamide | LCMS (ESI) m/z 409.1, [M + H]⁺. ¹H NMR (400 MHz, DMSO-d₆) δ 11.09 (s, 1H), 9.38 (s, 1H), 8.65 (s, 1H), 8.38-8.33 (m, 1H), 8.29 (s, 1H), 7.66-7.60 (m, 2H), 7.33 (t, J = 73.6 Hz, 1H), 7.29-7.22 (m, 2H), 3.02 (d, J = 4.4 Hz, 3H), 2.14- 2.03 (m, 1H), 0.95-0.80 (m, 4H). |
| Example 655 | 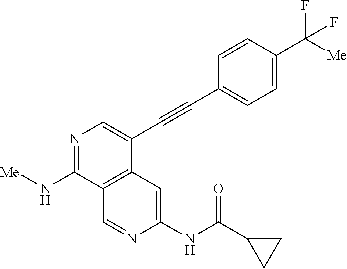 [SMILES: CNc1ncc(C#Cc2ccc(C(C)(F)F)cc2)c2cc(NC(=O)C3CC3)ncc12] N-(5-((4-(1,1- difluoroethyl)phenyl)ethynyl)-8- (methylamino)-2,7-naphthyridin-3- ylcyclopropanecarboxamide | LCMS (ESI) m/z 407.2, [M + H]⁺. ¹H NMR (400 MHz, DMSO-d₆) δ 11.09 (s, 1H), 9.38 (s, 1H), 8.67 (s, 1H), 8.43-8.37 (m, 1H), 8.31 (s, 1H), 7.73-7.61 (m, 4H), 3.03 (d, J = 4.4 Hz, 3H), 2.14-2.06 (m, 1H), 2.00 (t, J = 18.8 Hz, 3H), 0.95-0.83 (m, 4H). |
| Example 656 | 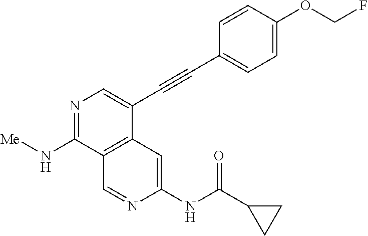 [SMILES: CNc1ncc(C#Cc2ccc(OCF)cc2)c2cc(NC(=O)C3CC3)ncc12] N-(5-((4- (fluoromethoxy)phenyl)ethynyl)-8- (methylamino)-2,7-naphthyridin-3- yl)cyclopropanecarboxamide | LCMS (ESI) m/z 391.1, [M + H]⁺. ¹H NMR (400 MHz, DMSO-d₆) δ 11.06 (s, 1H), 9.37 (s, 1H), 8.65 (s, 1H), 8.35-8.29 (m, 1H), 8.28 (s, 1H), 7.63-7.53 (m, 2H), 7.22- 7.17 (m, 2H), 5.92 (d, J = 54.0 Hz, 2H), 3.02 (d, J = 4.4 Hz, 3H), 2.15- 2.03 (m, 1H), 0.96-0.81 (m, 4H). |
| Example 657 | 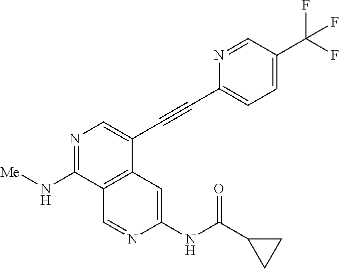 [SMILES: CNc1ncc(C#Cc2ccc(C(F)(F)F)cn2)c2cc(NC(=O)C3CC3)ncc12] N-(8-(methylamino)-5-((5- (trifluoromethyl)pyridin-2- yl)ethynyl)-2,7-naphthyridin-3- yl)cyclopropanecarboxamide | LCMS (ESI) m/z 412.1, [M + H]⁺. ¹H NMR (400 MHz, DMSO-d₆) δ 11.13 (s, 1H), 9.40 (s, 1H), 9.02 (d, J = 2.0 Hz, 1H), 8.65 (s, 1H), 8.57-8.50 (m, 1H), 8.42 (s, 1H), 8.33-8.27 (m, 1H), 7.82 (d, J = 8.4 Hz, 1H), 3.05 (d, J = 4.4 Hz, 3H), 2.14-2.05 (m, 1H), 0.95- 0.81 (m, 4H). |
| Example 658 | 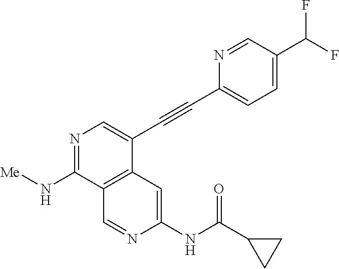 [SMILES: CNc1ncc(C#Cc2ccc(C(F)F)cn2)c2cc(NC(=O)C3CC3)ncc12] N-(5-((5-(difluoromethyl)pyridin-2- ylethynyl)-8-(methylamino)-2,7- naphthyridin-3- yl)cyclopropanecarboxamide | LCMS (ESI) m/z 394.1, [M + H]⁺. ¹H NMR (400 MHz, DMSO-d₆) δ 11.11 (s, 1H), 9.40 (s, 1H), 8.81 (s, 1H), 8.64 (s, 1H), 8.53-8.48 (m, 1H), 8.40 (s, 1H), 8.09 (d, J = 8.2 Hz, 1H), 7.76 (d, J = 8.2 Hz, 1H), 7.19 (d, J = 55.2 Hz, 1H), 3.05 (d, J = 4.4 Hz, 3H), 2.15-2.03 (m, 1H), 0.95-0.83 (m, 4H). |
| Example 659 | 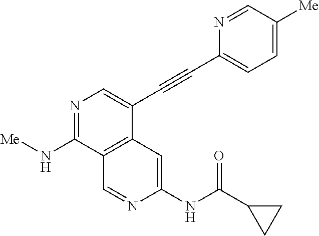 [SMILES: CNc1ncc(C#Cc2ccc(C)cn2)c2cc(NC(=O)C3CC3)ncc12] N-(8-(methylamino)-5-((5- methylpyridin-2-yl)ethynyl)-2,7- naphthyridin-3- yl)cyclopropanecarboxamide | LCMS (ESI) m/z 358.1, [M + H]⁺. ¹H NMR (400 MHz, DMSO-d₆) δ 11.09 (s, 1H), 9.38 (s, 1H), 8.62 (s, 1H), 8.46 (s, 1H), 8.43-8.36 (m, 1H), 8.33 (s, 1H), 7.69 (d, J = 8.0 Hz, 1H), 7.53 (d, J = 8.0 Hz, 1H), 3.03 (d, J = 4.4 Hz, 3H), 2.34 (s, 3H), 2.12-2.04 (m, 1H), 0.90- 0.83 (m, 4H). |
| Example 660 | 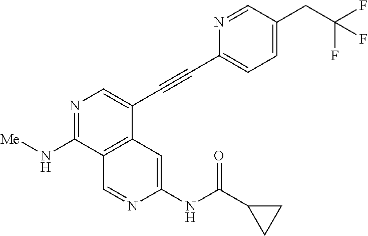 [SMILES: CNc1ncc(C#Cc2ccc(CC(F)(F)F)cn2)c2cc(NC(=O)C3CC3)ncc12] N-(8-(methylamino)-5-((5-(2,2,2- trifluoroethyl)pyridin-2-yl)ethynyl)- 2,7-naphthyridin-3- yl)cyclopropanecarboxamide | LCMS (ESI) m/z 426.1 [M + H]⁺. ¹H NMR (400 MHz, DMSO-d₆) δ 11.11 (s, 1H), 9.39 (s, 1H), 8.65- 8.58 (m, 2H), 8.50-8.40 (m, 1H), 8.36 (s, 1H), 7.88 (d, J = 8.0 Hz, 1H), 7.66 (d, J = 8.0 Hz, 1H), 3.89- 3.75 (m, 2H), 3.04 (d, J = 4.4 Hz, 3H), 2.14-2.02 (m, 1H), 0.94- 0.80 (m, 4H). |
| Example 661 | 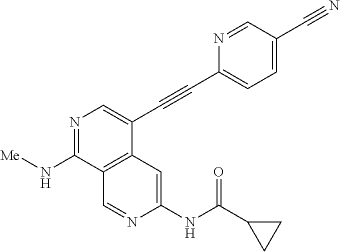 [SMILES: CNc1ncc(C#Cc2ccc(C#N)cn2)c2cc(NC(=O)C3CC3)ncc12] N-(5-((5-cyanopyridin-2-yl)ethynyl)- 8-(methylamino)-2,7-naphthyridin-3- yl)cyclopropanecarboxamide | LCMS (ESI) m/z 369.1, [M + H]⁺. ¹H NMR (400 MHz, DMSO-d₆) δ 11.13 (s, 1H), 9.40 (s, 1H), 9.04 (d, J = 2.0 Hz, 1H), 8.63 (s, 1H), 8.60-8.53 (m, 1H), 8.43 (s, 1H), 8.38 (dd, J = 8.4, 2.0 Hz, 1H), 7.77 (d, J = 8.0 Hz, 1H), 3.05 (d, J = 4.4 Hz, 3H), 2.15-2.04 (m, 1H), 0.95-0.81 (m, 4H). |
| Example 662 | 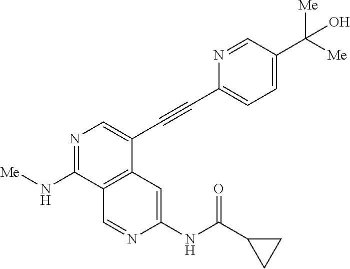 [SMILES: CNc1ncc(C#Cc2ccc(C(C)(C)O)cn2)c2cc(NC(=O)C3CC3)ncc12] N-(5-((5-(2-hydroxypropan-2- yl)pyridin-2-yl)ethynyl)-8- (methylamino)-2,7-naphthyridin-3- yl)cyclopropanecarboxamide | LCMS (ESI) m/z 402.2, [M + H]⁺. ¹H NMR (400 MHz, DMSO-d₆) δ 11.10 (s, 1H), 9.39 (s, 1H), 8.71 (d, J = 2.0 Hz, 1H), 8.63 (s, 1H), 8.43-8.39 (m, 1H), 8.34 (s, 1H), 7.90 (dd, J = 8.0, 2.0 Hz, 1H), 7.57 (d, J = 8.0 Hz, 1H), 5.39 (s, 1H), 3.03 (d, J = 4.4 Hz, 3H), 2.11- 2.05 (m, 1H), 1.48 (s, 6H), 0.91- 0.86 (m, 4H). |
| Example 663 | 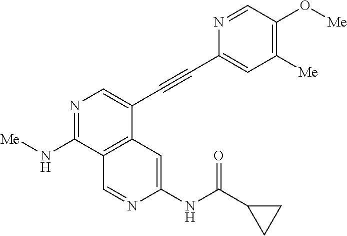 [SMILES: CNc1ncc(C#Cc2cc(C)c(OC)cn2)c2cc(NC(=O)C3CC3)ncc12] N-(5-((5-methoxy-4-methylpyridin-2- yl)ethynyl)-8-(methylamino)-2,7- naphthyridin-3- yl)cyclopropanecarboxamide | LCMS (ESI) m/z 388.2, [M + H]⁺. ¹H NMR (400 MHz, DMSO-d₆) δ 11.10 (s, 1H), 9.38 (s, 1H), 8.65 (s, 1H), 8.41-8.36 (m, 1H), 8.30 (s, 1H), 8.28 (s, 1H), 7.55 (s, 1H), 3.95 (s, 3H), 3.03 (d, J = 4.4 Hz, 3H), 2.20 (s, 3H), 2.14-2.03 (m, 1H), 0.93-0.80 (m, 4H). |
| Example 664 | 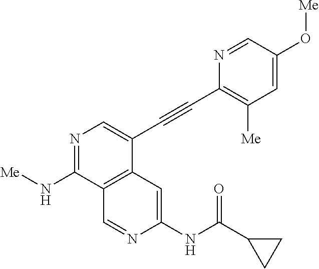 [SMILES: CNc1ncc(C#Cc2ncc(OC)cc2C)c2cc(NC(=O)C3CC3)ncc12] N-(5-((5-methoxy-3-methylpyridin-2- yl)ethynyl)-8-(methylamino)-2,7- naphthyridin-3- yl)cyclopropanecarboxamide | LCMS (ESI) m/z 388.2, [M + H]⁺. ¹H NMR (400 MHz, DMSO-d₆) δ 11.07 (s, 1H), 9.39 (s, 1H), 8.64 (s, 1H), 8.39-8.32 (m, 1H), 8.32 (s, 1H), 8.15 (d, J = 2.4 Hz, 1H), 7.39 (d, J = 2.4 Hz, 1H), 3.87 (s, 3H), 3.03 (d, J = 4.4 Hz, 3H), 2.54 (s, 3H), 2.13-2.04 (m, 1H), 0.89- 0.82 (m, 4H). |
| Example 665 | 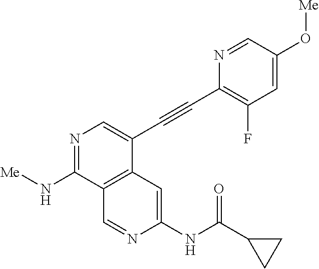 [SMILES: CNc1ncc(C#Cc2ncc(OC)cc2F)c2cc(NC(=O)C3CC3)ncc12] N-(5-((3-fluoro-5-methoxypyridin-2- yl)ethynyl)-8-(methylamino)-2,7- naphthyridin-3- yl)cyclopropanecarboxamide | LCMS (ESI) m/z 392.1, [M + H]⁺. ¹H NMR (400 MHz, DMSO-d₆) δ 11.06 (s, 1H), 9.39 (s, 1H), 8.57 (s, 1H), 8.48-8.41 (m, 1H), 8.33 (s, 1H), 8.24 (d, J = 2.4 Hz, 1H), 7.61 (dd, J = 10.8, 2.4 Hz, 1H), 3.92 (s, 3H), 3.04 (d, J = 4.4 Hz, 3H), 2.13- 2.02 (m, 1H), 0.90-0.80 (m, 4H). |
| Example 666 | 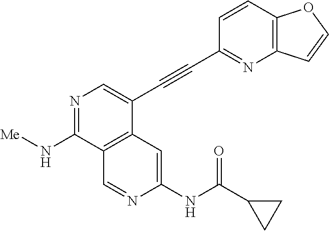 [SMILES: CNc1ncc(C#Cc2ccc3occc3n2)c2cc(NC(=O)C3CC3)ncc12] N-(5-(furo[3,2-b]pyridin-5-ylethynyl)- 8-(methylamino)-2,7-naphthyridin-3- yl)cyclopropanecarboxamide | LCMS (ESI) m/z 384.1, [M + H]⁺. ¹H NMR (400 MHz, DMSO-d₆) δ 11.11 (s, 1H), 9.39 (s, 1H), 8.65 (s, 1H), 8.45-8.35 (m, 3H), 8.16- 8.11 (m, 1H), 7.60 (d, J = 8.4 Hz, 1H), 7.17 (d, J = 2.0 Hz, 1H), 3.04 (d, J = 4.4 Hz, 3H), 2.13-2.05 (m, 1H), 0.95-0.80 (m, 4H). |
| Example 667 | 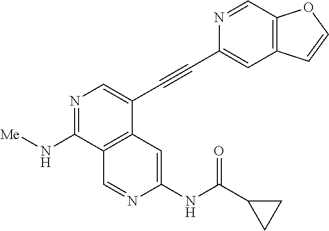 [SMILES: CNc1ncc(C#Cc2cc3ccoc3cn2)c2cc(NC(=O)C3CC3)ncc12] N-(5-(furo[2,3-c]pyridin-5-ylethynyl)- 8-(methylamino)-2,7-naphthyridin-3- yl)cyclopropanecarboxamide | LCMS (ESI) m/z 384.1, [M+H]+. 1H NMR (400 MHZ, DMSO-d6) 8 11.09 (s, 1H), 9.39 (s, 1H), 8.99 (s, 1H), 8.65 (s, 1H), 8.41-8.30 (m, 3H), 8.00 (s, 1H), 7.12 (s, 1H), 3.04 (d, J = 4.4 Hz, 3H), 2.14- 2.06 (m, 1H), 0.93-0.83 (m, 4H). |
| Example 668 | 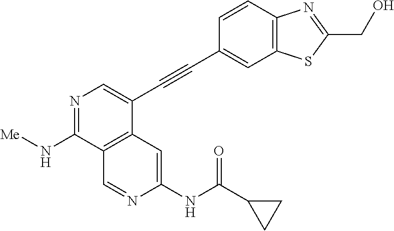 [SMILES: CNc1ncc(C#Cc2ccc3nc(CO)sc3c2)c2cc(NC(=O)C3CC3)ncc12] N-(5-((2- (hydroxymethyl)benzo[d]thiazol-6- yl)ethynyl)-8-(methylamino)-2,7- naphthyridin-3- yl)cyclopropanecarboxamide | LCMS (ESI) m/z 430.1, [M + H]⁺. ¹H NMR (400 MHz, DMSO-d₆) δ 11.11 (s, 1H), 9.39 (s, 1H), 8.70 (s, 1H), 8.40-8.33 (m, 2H), 8.31 (s, 1H), 7.97 (d, J = 8.4 Hz, 1H), 7.69- 7.62 (m, 1H), 6.32 (t, J = 6.0 Hz, 1H), 4.89 (d, J = 6.0 Hz, 2H), 3.03 (d, J = 4.4 Hz, 3H), 2.15-2.04 (m, 1H), 0.95-0.81 (m, 4H). |
| Example 669 | 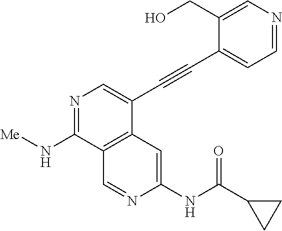 [SMILES: CNc1ncc(C#Cc2ccncc2CO)c2cc(NC(=O)C3CC3)ncc12] N-(5-((3-(hydroxymethyl)pyridin-4- yl)ethynyl)-8-(methylamino)-2,7- naphthyridin-3- yl)cyclopropanecarboxamide | LCMS (ESI) m/z 374.1, [M + H]⁺. ¹H NMR (400 MHz, DMSO-d₆) δ 11.11 (s, 1H), 9.39 (s, 1H), 8.70 (s, 1H), 8.63 (s, 1H), 8.51 (d, J = 5.2 Hz, 1H), 8.50-8.41 (m, 1H), 8.36 (s, 1H), 7.44 (d, J = 5.2 Hz, 1H), 5.35 (t, J = 5.6 Hz, 1H), 4.83 (d, J = 5.6 Hz, 2H), 3.04 (d, J = 4.4 Hz, 3H), 2.18-2.01 (m, 1H), 0.99- 0.79 (m, 4H). |
| Example 670 | 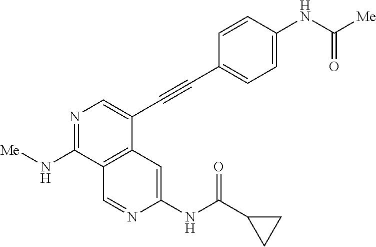 [SMILES: CNc1ncc(C#Cc2ccc(NC(C)=O)cc2)c2cc(NC(=O)C3CC3)ncc12] N-(5-((4-acetamidophenyl)ethynyl)-8- (methylamino)-2,7-naphthyridin-3- yl)cyclopropanecarboxamide | LCMS (ESI) m/z 400.1, [M + H]⁺. ¹H NMR (400 MHz, DMSO-d₆) δ 11.07 (s, 1H), 10.12 (s, 1H), 9.37 (s, 1H), 8.64 (s, 1H), 8.32-8.26 (m, 1H), 8.25 (s, 1H), 7.65 (d, J = 8.8 Hz, 2H), 7.49 (d, J = 8.8 Hz, 2H), 3.02 (d, J = 4.4 Hz, 3H), 2.12- 2.08 (m, 1H), 2.08 (s, 3H), 0.98- 0.81 (m, 4H). |
| Example 671 | 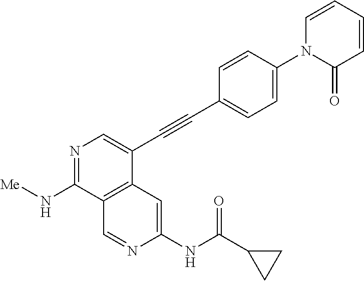 [SMILES: CNc1ncc(C#Cc2ccc(-n3ccccc3=O)cc2)c2cc(NC(=O)C3CC3)ncc12] N-(8-(methylamino)-5-((4-(2- oxopyridin-1(2H)-yl)phenyl)ethynyl)- 2,7-naphthyridin-3- yl)cyclopropanecarboxamide | LCMS (ESI) m/z 436.2, [M + H]⁺. ¹H NMR (400 MHz, DMSO-d₆) δ 11.09 (s, 1H), 9.39 (s, 1H), 8.69 (s, 1H), 8.44-8.34 (m, 1H), 8.32 (s, 1H), 7.80-7.62 (m, 3H), 7.59- 7.46 (m, 3H), 6.51 (d, J = 9.2 Hz, 1H), 6.41-6.29 (m, 1H), 3.04 (d, J = 4.4 Hz, 3H), 2.14-2.04 (m, 1H), 0.99-0.79 (m, 4H). |
| Example 672 | 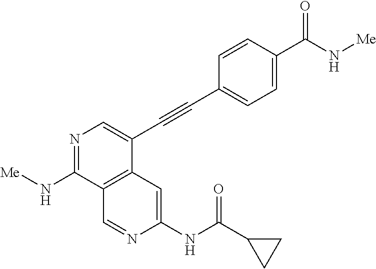 [SMILES: CNC(=O)c1ccc(C#Cc2cnc(NC)c3cnc(NC(=O)C4CC4)cc23)cc1] 4-((6-(cyclopropanecarboxamido)-1- (methylamino)-2,7-naphthyridin-4- yl)ethynyl)-N-methylbenzamide | LCMS (ESI) m/z 400.2, [M + H]⁺. ¹H NMR (400 MHz, DMSO-d₆) δ 11.09 (s, 1H), 9.38 (s, 1H), 8.68 (s, 1H), 8.55-8.47 (m, 1H), 8.40- 8.34 (m, 1H), 8.31 (s, 1H), 7.90 (d, J = 8.1 Hz, 2H), 7.64 (d, J = 8.1 Hz, 2H), 3.03 (d, J = 4.4 Hz, 3H), 2.81 (d, J = 4.5 Hz, 3H), 2.15- 2.04 (m, 1H), 0.96-0.81 (m, 4H). |
| Example 673 | 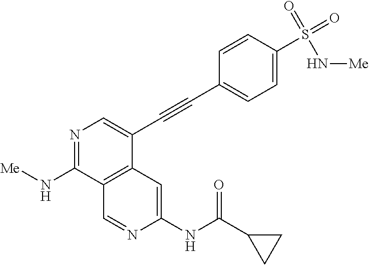 [SMILES: CNc1ncc(C#Cc2ccc(S(=O)(=O)NC)cc2)c2cc(NC(=O)C3CC3)ncc12] N-(8-(methylamino)-5-((4-(N- methylsulfamoyl)phenyl)ethynyl)-2,7- naphthyridin-3- yl)cyclopropanecarboxamide | LCMS (ESI) m/z 436.1, [M + H]⁺. ¹H NMR (400 MHz, DMSO-d₆) δ 11.12 (s, 1H), 9.39 (s, 1H), 8.67 (s, 1H), 8.46-8.40 (m, 1H), 8.34 (s, 1H), 7.87-7.81 (m, 2H), 7.80- 7.73 (m, 2H), 7.57-7.51 (m, 1H), 3.04 (d, J = 4.4 Hz, 3H), 2.46 (d, J = 4.8 Hz, 3H), 2.14-2.05 (m, 1H), 0.94-0.82 (m, 4H). |
| Example 674 | 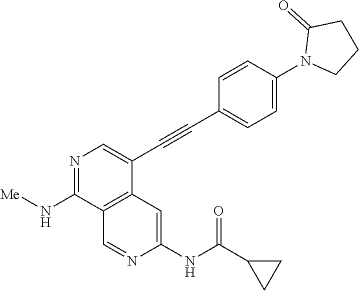 [SMILES: CNc1ncc(C#Cc2ccc(N3CCCC3=O)cc2)c2cc(NC(=O)C3CC3)ncc12] N-(8-(methylamino)-5-((4-(2- oxopyrrolidin-1-yl)phenyl)ethynyl)- 2,7-naphthyridin-3- yl)cyclopropanecarboxamide | LCMS (ESI) m/z 426.2, [M + H]⁺. ¹H NMR (400 MHz, DMSO-d₆) δ 11.08 (s, 1H), 9.37 (s, 1H), 8.66 (s, 1H), 8.34-8.29 (m, 1H), 8.27 (s, 1H), 7.76 (d, J = 8.8 Hz, 2H), 7.56 (d, J = 8.8 Hz, 2H), 3.88 (t, J = 7.2 Hz, 2H), 3.02 (d, J = 4.4 Hz, 3H), 2.55-2.50 (m, 2H), 2.12-2.04 (m, 3H), 0.94-0.81 (m, 4H). |
| Example 675 | 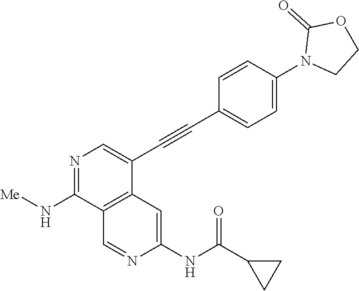 [SMILES: CNc1ncc(C#Cc2ccc(N3CCOC3=O)cc2)c2cc(NC(=O)C3CC3)ncc12] N-(8-(methylamino)-5-((4-(2- oxooxazolidin-3-yl)phenyl)ethynyl)- 2,7-naphthyridin-3- yl)cyclopropanecarboxamide | LCMS (ESI) m/z 428.2, [M + H]⁺. ¹H NMR (400 MHz, DMSO-d₆) δ 11.08 (s, 1H), 9.38 (s, 1H), 8.67 (s, 1H), 8.35-8.29 (m, 1H), 8.27 (s, 1H), 7.72-7.54 (m, 4H), 4.47 (d, J = 8.0 Hz, 2H), 4.11 (d, J = 8.0 Hz, 2H), 3.02 (d, J = 4.4 Hz, 3H), 2.13-2.04 (m, 1H), 0.95-0.80 (m, 4H). |
| Example 676 | 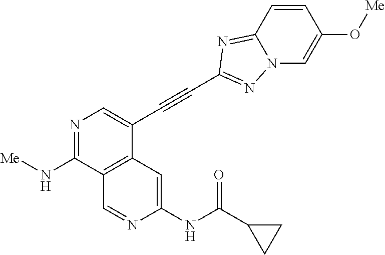 [SMILES: CNc1ncc(C#Cc2nc3ccc(OC)cn3n2)c2cc(NC(=O)C3CC3)ncc12] N-(5-((6-methoxy-[1,2,4]triazolo[1,5- a]pyridin-2-yl)ethynyl)-8- (methylamino)-2,7-naphthyridin-3- yl)cyclopropanecarboxamide | LCMS (ESI) m/z 414.2, [M + H]⁺. ¹H NMR (400 MHz, DMSO-d₆) δ 11.11 (s, 1H), 9.40 (s, 1H), 8.71 (d, J = 2.0 Hz, 1H), 8.59 (s, 1H), 8.51-8.47 (m, 1H), 8.41 (s, 1H), 7.77 (d, J = 9.6 Hz, 1H), 7.52 (dd, J = 9.6, 2.4 Hz, 1H), 3.89 (s, 3H), 3.05 (d, J = 4.4 Hz, 3H), 2.10- 2.06 (m, 1H), 0.87-0.84 (m, 4H). |

### Example 677: Synthesis of N-(8-(methylamino)-5-((4-(oxetan-3-ylmethoxy)phenyl)ethynyl)-2,7-naphthyridin-3-yl)cyclopropanecarboxamide

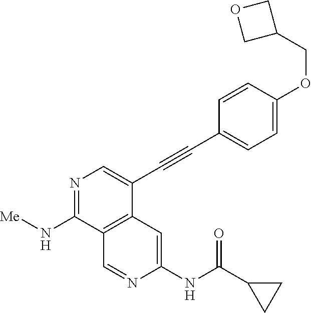 [SMILES: CNc1ncc(C#Cc2ccc(OCC3COC3)cc2)c2cc(NC(=O)C3CC3)ncc12]

### Step 1: 3-((4-bromophenoxy)methyl)oxetane

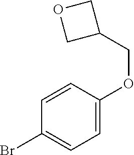 [SMILES: Brc1ccc(OCC2COC2)cc1]

A solution of 4-bromophenol (500 mg; 2.890 mmol; 1.00 eq.), 3-(bromomethyl)oxetane (872.8 mg; 5.780 mmol; 2.00 eq.) and Cs₂CO₃ (2824.8 mg; 8.670 mmol; 3.00 eq.) in DMF (6 mL) was stirred at 60° C. for 2 hours. The desired product was detected via LCMS. The solution was diluted with water (10 mL) and extracted with EtOAc (3×50 mL). The combined organic layers were washed with brine (3×25 mL) and dried over anhydrous Na₂SO₄. After filtration, the filtrate was concentrated under reduced pressure. The residue was purified by flash chromatography on silica gel column using 10-30% of EtOAc in petroleum ether as eluent to provide 3-((4-bromophenoxy)methyl)oxetane as a white solid (574.0 mg, 81.7%). LCMS (ESI) m/z 243.1, [M+H]⁺.

### Step 2: N-(8-(methylamino)-5-((4-(oxetan-3-ylmethoxy)phenyl)ethynyl)-2,7-naphthyridin-3-yl)cyclopropanecarboxamide

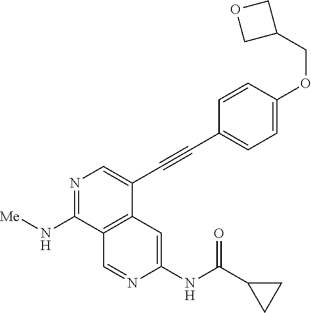 [SMILES: CNc1ncc(C#Cc2ccc(OCC3COC3)cc2)c2cc(NC(=O)C3CC3)ncc12]

N-(8-(methylamino)-5-((4-(oxetan-3-ylmethoxy)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-((4-bromophenoxy)methyl)oxetane as the starting material. LCMS (ESI) m/z 429.2, [M+H]⁺. ¹H NMR (400 MHz, DMSO-d₆) δ 11.07 (s, 1H), 9.37 (s, 1H), 8.65 (s, 1H), 8.38-8.26 (m, 1H), 8.25 (s, 1H), 7.50 (d, J=8.8 Hz, 2H), 7.04 (d, J=8.8 Hz, 2H), 4.75-4.71 (m, 2H), 4.46-4.45 (m, 2H), 4.27 (d, J=6.8 Hz, 2H), 3.45-3.38 (m, 1H), 3.02 (d, J=4.4 Hz, 3H), 2.11-2.06 (m, 1H), 0.92-0.90 (m, 4H).

### Example 678: Synthesis of N-(8-(methylamino)-5-((5-(oxetan-3-ylmethoxy)pyridin-2-yl)ethynyl)-2,7-naphthyridin-3-yl)cyclopropanecarboxamide

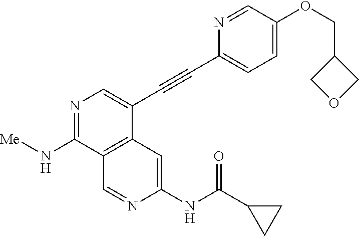 [SMILES: CNc1ncc(C#Cc2ccc(OCC3COC3)cn2)c2cc(NC(=O)C3CC3)ncc12]

### Step 1: 2-bromo-5-(oxetan-3-ylmethoxy)pyridine

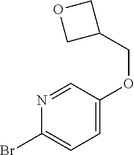 [SMILES: Brc1ccc(OCC2COC2)cn1]

A solution of 6-bromopyridin-3-ol (100.0 mg; 0.575 mmol; 1.00 eq.), 3-(bromomethyl)oxetane (173.5 mg; 1.149 mmol; 2.00 eq.) and Cs₂CO₃ (561.7 mg; 1.724 mmol; 3.00 eq.) in DMF (2 mL) was stirred at 50° C. for 2 hours under nitrogen atmosphere. The solution was diluted with water (10 mL) and extracted with EtOAc (3×20 mL). The combined organic layers were washed with brine (3×10 mL) and dried over anhydrous Na₂SO₄. After filtration, the filtrate was concentrated under reduced pressure. The residue was purified by flash chromatography on silica gel column using 20-40% of EtOAc in petroleum ether as eluent to provide 2-bromo-5-(oxetan-3-ylmethoxy)pyridine as a white solid (80.0 mg, 57.0%). LCMS (ESI) m/z 244.1, [M+H]⁺.

### Step 2: N-(8-(methylamino)-5-((5-(oxetan-3-ylmethoxy)pyridin-2-yl)ethynyl)-2,7-naphthyridin-3-yl)cyclopropanecarboxamide

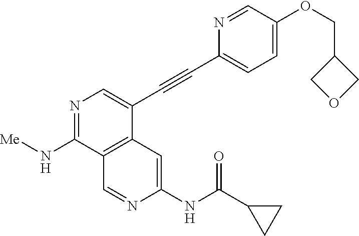 [SMILES: CNc1ncc(C#Cc2ccc(OCC3COC3)cn2)c2cc(NC(=O)C3CC3)ncc12]

N-(8-(methylamino)-5-((5-(oxetan-3-ylmethoxy)pyridin-2-yl)ethynyl)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 2-bromo-5-(oxetan-3-ylmethoxy)pyridine as the starting material. LCMS (ESI) m/z 430.2, [M+H]⁺. ¹H NMR (400 MHz, DMSO-d₆) δ 11.09 (s, 1H), 9.38 (s, 1H), 8.62 (s, 1H), 8.40-8.33 (m, 2H), 8.32 (s, 1H), 7.60 (d, J=8.8 Hz, 1H), 7.55-7.50 (m, 1H), 4.77-4.69 (m, 2H), 4.49-4.43 (m, 2H), 4.35 (d, J=6.8 Hz, 2H), 3.50-3.39 (m, 1H), 3.03 (d, J=4.4 Hz, 3H), 2.12-2.05 (m, 1H), 0.91-0.81 (m, 4H).

### Example 679: Synthesis of N-(5-((4-((1-acetylazetidin-3-yl)methoxy)phenyl)ethynyl)-8-(methylamino)-2,7-naphthyridin-3-yl)cyclopropanecarboxamide

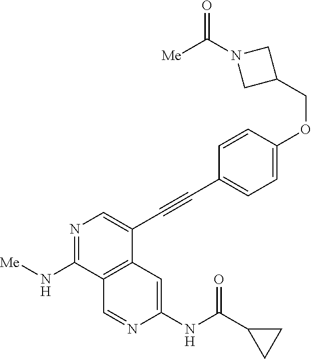 [SMILES: CNc1ncc(C#Cc2ccc(OCC3CN(C(C)=O)C3)cc2)c2cc(NC(=O)C3CC3)ncc12]

### Step 1: 1-(3-((4-bromophenoxy)methyl)azetidin-1-yl)ethan-1-one

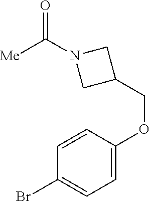 [SMILES: CC(=O)N1CC(COc2ccc(Br)cc2)C1]

A solution of 4-bromophenol (500.0 mg; 2.890 mmol; 1.00 eq.), 1-(3-(hydroxymethyl)azetidin-1-yl)ethan-1-one (298.6 mg; 2.312 mmol; 0.80 eq.), DIAD (587.2 mg; 2.904 mmol; 1.00 eq.) and PPh₃ (833.8 mg; 3.1.79 mmol; 1.10 eq.) in THF (12.5 mL) was stirred at room temperature for 14 hours under nitrogen atmosphere. The desired product was detected via LCMS. The resulting mixture was concentrated under vacuum and purified by flash chromatography on pre-packed C18 column using 30-50% of MeOH in water (10 mmol/L NH₄HCO₃) as eluent to provide 1-(3-((4-bromophenoxy)methyl)azetidin-1-yl)ethan-1-one as a yellow solid (215.0 mg, 19.9%).

### Step 2: N-(5-((4-((1-acetylazetidin-3-yl)methoxy)phenyl)ethynyl)-8-(methylamino)-2,7-naphthyridin-3-yl)cyclopropanecarboxamide

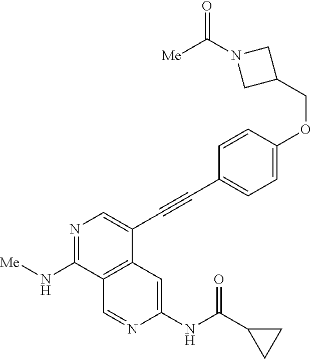 [SMILES: CNc1ncc(C#Cc2ccc(OCC3CN(C(C)=O)C3)cc2)c2cc(NC(=O)C3CC3)ncc12]

N-(5-((4-((1-acetylazetidin-3-yl)methoxy)phenyl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and i-(3-((4-bromophenoxy)methyl)azetidin-1-yl)ethan-1-one as the starting material. LCMS (ESI) m/z 470.2, [M+H]⁺. ¹H NMR (400 MHz, DMSO-d₆) δ 11.07 (s, 1H), 9.37 (s, 1H), 8.65 (s, 1H), 8.32-8.27 (m, 1H), 8.24 (s, 1H), 7.51-7.49 (m, 2H), 7.04-7.02 (m, 2H), 4.26-4.18 (m, 3H), 3.97-3.92 (m, 2H), 3.67-3.63 (m, 1H), 3.06-2.96 (m, 4H), 2.08-2.07 (m, 1H), 1.76 (s, 3H), 0.90-0.87 (m, 4H).

### Example 680: Synthesis of N-(8-(methylamino)-5-(pyrazolo[1,5-a]pyridin-2-ylethynyl)-2,7-naphthyridin-3-yl)cyclopropanecarboxamide

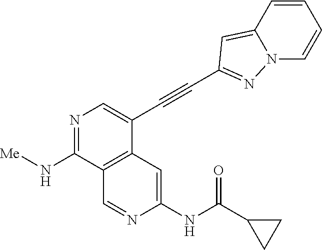 [SMILES: CNc1ncc(C#Cc2cc3ccccn3n2)c2cc(NC(=O)C3CC3)ncc12]

### Step 1: pyrazolo[1,5-a]pyridin-2-yl trifluoromethanesulfonate

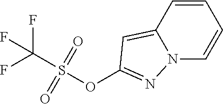 [SMILES: O=S(=O)(Oc1cc2ccccn2n1)C(F)(F)F]

To a stirred solution of pyrazolo[1,5-a]pyridin-2-ol (200.0 mg; 1.490 mmol; 1.00 eq.) in a mixture solvent of THF/DMF (1:1, 6 mL) was added NaH (60% dispersion in mineral oil, 59.6 mg; 1.490 mmol; 1.00 eq.) at 0° C. under nitrogen atmosphere. After stirring for 10 minutes, to the above solution was added 1,1,1-trifluoro-N-phenyl-N-((trifluoromethyl)sulfonyl)methanesulfonamide (639.1 mg; 1.782 mmol; 1.20 eq.) dropwise at 0° C. The resulting mixture was stirred at room temperature for 1 hour. The desired product was detected via LCMS. The reaction was quenched by the addition of water/ice (10 mL) at 0° C. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by flash chromatography on silica gel column using 0-1.5% of EtOAc in petroleum ether as eluent to provide pyrazolo[1,5-a]pyridin-2-yl trifluoromethanesulfonate as off-white oil (400.1 mg, 96.0%). LCMS (ESI) m/z 267.0, [M+H]⁺.

### Step 2: N-(8-(methylamino)-5-(pyrazolo[1,5-a]pyridin-2-ylethynyl)-2,7-naphthyridin-3-yl)cyclopropanecarboxamide

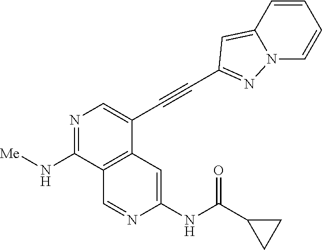 [SMILES: CNc1ncc(C#Cc2cc3ccccn3n2)c2cc(NC(=O)C3CC3)ncc12]

N-(8-(methylamino)-5-(pyrazolo[1,5-a]pyridin-2-ylethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and pyrazolo[1,5-a]pyridin-2-yl trifluoromethanesulfonate as the starting material. LCMS (ESI) m/z 383.2, [M+H]⁺. ¹H NMR (400 MHz, DMSO-d₆) δ 11.10 (s, 1H), 9.39 (s, 1H), 8.73-8.66 (m, 1H), 8.61 (s, 1H), 8.44-8.37 (m, 1H), 8.35 (s, 1H), 7.75-7.69 (m, 1H), 7.33-7.24 (m, 1H), 7.02-6.95 (m, 1H), 6.87 (s, 1H), 3.04 (d, J=4.4 Hz, 3H), 2.15-2.03 (m, 1H), 0.96-0.82 (m, 4H).

### Example 681: Synthesis of N-(5-((1-isopropyl-1H-benzo[d][1,2,3]triazol-6-yl)ethynyl)-8-(methylimino)-2,7-naphthyridin-3-yl)cyclopropanecarboxamide

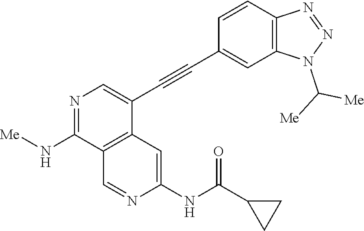 [SMILES: CNc1ncc(C#Cc2ccc3nnn(C(C)C)c3c2)c2cc(NC(=O)C3CC3)ncc12]

### Step 1: 5-bromo N-isopropyl-2-nitroaniline

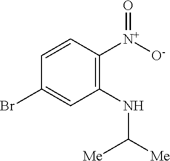 [SMILES: CC(C)Nc1cc(Br)ccc1[N+](=O)[O-]]

To a solution of 4-bromo-2-fluoro-1-nitrobenzene (2.00 g; 9.091 mmol; 1.00 eq.) and propan-2-amine (0.60 g; 10.000 mmol; 1.10 eq.) in DMF (20 mL) was added K₂CO₃ (2.50 g; 18.182 mmol; 2.00 eq.) at room temperature. The resulting mixture was stirred at room temperature for 12 hours. The desired product was detected via LCMS. The resulting mixture was diluted with water (20 mL) and extracted with EtOAc (3×20 mL). The combined organic layers were washed with brine (3×20 mL) and dried over anhydrous Na₂SO₄. After filtration, the filtrate was concentrated under reduced pressure to afford 5-bromo-N-isopropyl-2-nitroaniline as an orange solid (2.00 g, 84.0%). LCMS (ESI) m/z 259.0, [M+H]⁺.

### Step 2: 5-bromo-N¹-isopropylbenzene-1,2-diamine

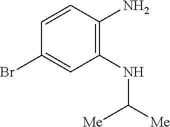 [SMILES: CC(C)Nc1cc(Br)ccc1N]

A solution of 5-bromo-N-isopropyl-2-nitroaniline (1.00 g; 3.859 mmol; 1.00 eq.) and Fe (2.20 g; 38.590 mmol; 10.00 eq.) in AcOH (10 mL) was stirred at 70° C. for 1 hour under nitrogen atmosphere. The desired product was detected via LCMS. The mixture was allowed to cool down to room temperature. The resulting mixture was diluted with water (20 mL). The mixture was neutralized to pH=7 with saturated NaHCO₃ solution and extracted with EtOAc (3×20 mL). The combined organic layers were washed with water (3×20 mL) and dried over anhydrous Na₂SO₄. After filtration, the filtrate was concentrated under reduced pressure to afford in 5-bromo-N¹-isopropylbenzene-1,2-diamine as a brown oil (600.0 mg, 68.0%). LCMS (ESI) m/z 229.0, [M+H]⁺.

### Step 3: 6-bromo-1-isopropyl-1H-benzo[d][1,2,3]triazole

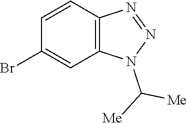 [SMILES: CC(C)n1nnc2ccc(Br)cc21]

To a solution of 5-bromo N^{f}-isopropylbenzene-1,2-diamine (300.0 mg; 1.309 mmol; 1.00 eq.) in HCl (12 M, 4 mL) was added a solution of NaNO₂ (117.4 mg; 1.702 mmol; 1.30 eq.) in water (1 mL) dropwise at 0° C. The resulting solution was stirred at room temperature for 1 hour. The desired product was detected via LCMS. The resulting mixture was diluted with water (20 mL). The mixture was neutralized to pH=7 with a saturated NaHCO₃ solution. The aqueous phase was extracted with EtOAc (3×50 mL). The combined organic layers were washed with water (3 1.5×20 mL) and dried over anhydrous Na₂SO₄. The solvent was concentrated under reduced pressure. The residue was purified by flash chromatography on silica gel column using 0-5% of MeOH in CH₂Cl₂ to afford 6-bromo-1-isopropyl-1H-benzo[d][1,2,3]triazole as a brown solid (240.0 mg, 76.0%). LCMS (ER) m/z 240.0, [M+H]⁺.

### Step 4: N-(5-((1-isopropyl-1H-benzo[d][1,2,3]triazol-6-yl)ethynyl)-8-(methylamino)-2,7-naphthyridin-3-yl)cyclopropanecarboxamide

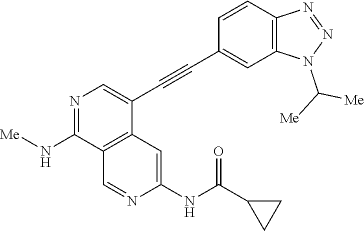 [SMILES: CNc1ncc(C#Cc2ccc3nnn(C(C)C)c3c2)c2cc(NC(=O)C3CC3)ncc12]

N-(5-((1-isopropyl-1H-benzo[d][1,2,3]triazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-1-isopropyl-1H-benzo[d][1,2,3]triazole as the starting material. LCMS (ESI) m/z 426.2, [M+H]⁺. ¹H NMR (400 MHz, DMSO-d₆) δ 11.16 (s, 1H), 9.40 (s, 1H), 8.84 (s, 1H), 8.45-8.40 (m, 1H), 8.33 (s, 1H), 8.24 (s, 1H), 8.10 (d, J=8.8 Hz, 1H), 7.53 (dd, J=8.8, 1.2 Hz, 1H), 5.35-5.24 (m, 1H), 3.04 (d, J=4.4 Hz, 3H), 2.17-2.07 (m, 1H), 1.69 (d, J=6.8 Hz, 6H), 0.99-0.77 (m, 4H).

### Example 682: Synthesis of N-(5-((1-(2-methoxyethyl)-1H-benzo[d][1,2,3]triazol-6-yl)ethynyl)-8-(methylamino)-2,7-naphthyridin-3-yl)cyclopropanecarboxamide

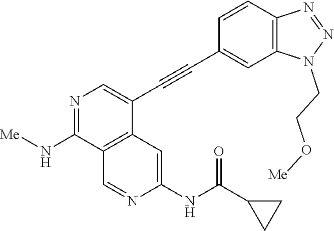 [SMILES: CNc1ncc(C#Cc2ccc3nnn(CCOC)c3c2)c2cc(NC(=O)C3CC3)ncc12]

### Step 1: 5-bromo-N-(2-methoxyethyl)-2-nitroaniline

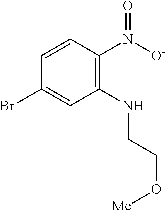 [SMILES: COCCNc1cc(Br)ccc1[N+](=O)[O-]]

To a solution of 4-bromo-2-fluoro-1-nitrobenzene (2.01 g; 9.091 mmol; 1.00 eq.) and 2-methoxyethan-1-amine (0.71 g; 10.000 mmol; 1.10 eq.) in DMF (5 mL) was added K₂CO₃ (2.50 g; 18.182 mmol; 2.00 eq.) at room temperature. The resulting mixture was stirred at room temperature for 12 hours. The desired product was detected via LCMS. The resulting mixture was diluted with water (1.0 mL) and extracted with EtOAc (3×10 mL). The combined organic layers were washed with water (3×10 mL), dried over anhydrous Na₂SO₄. After filtration, the filtrate was concentrated under reduced pressure to afford 5-bromo-N-(2-methoxyethyl)-2-nitroaniline as an orange solid (2.00 g, 79.0%). LCMS (ESI) m/z 275.0, [M+H]⁺.

### Step 2: 5-bromo-N¹-(2-methoxyethyl)benzene-1,2-diamine

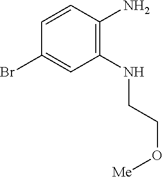 [SMILES: COCCNc1cc(Br)ccc1N]

A solution of 5-bromo-N-(2-methoxyethyl)-2-nitroaniline (1.00 g; 3.635 mmol; 1.00 eq.) and Fe (2029.9 mg; 36.350 mmol; 10.00 eq.) in AcOH (10 mL) was stirred at 70° C. for 3 hours under nitrogen atmosphere. The desired product was detected via LCMS. The mixture was allowed to cool down to room temperature. The mixture was diluted with water (10 mL) and neutralized to pH=7 with saturated NaHCO₃ solution. The aqueous phase was extracted with EtOAc (3×50 mL). The combined organic layers were washed with water (3×30 mL), dried over anhydrous Na₂SO₄. After filtration, the filtrate was concentrated under reduced pressure to afford 5-bromo-N¹-(2-methoxyethyl)benzene-1,2-diamine as a brown oil (600.0 mg, 67.0%). LCMS (ESI) m/z 245.0, [M+H]⁴.

### Step 3: 6-bromo-1-(2-methoxyethyl)-1H-benzo[d][1,2,3]triazole

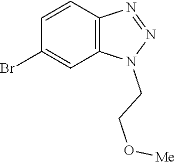 [SMILES: COCCn1nnc2ccc(Br)cc21]

To a solution of 5-bromo-N¹-(2-methoxyethyl)benzene-1,2-diamine (200.0 mg; 0.816 mmol; 1.00 eq.) in HCl (4 M, 4 mL) was added a solution of NaNO₂ (73.2 mg; 1.061 mmol; 1.30 eq.) in water (1 mL) at 0° C. The resulting solution was stirred at room temperature for 1 hour. The desired product was detected via LCMS. The resulting mixture was diluted with water (20 mL). The mixture was neutralized to pH=7 with saturated NaHCO₃ solution. The aqueous phase was extracted with EtOAc (3×50 mL). The combined organic layers were washed with water (3×50 mL) and dried over anhydrous Na₂SO₄. The organic layer was concentrated under reduced pressure to afford 6-bromo-1-(2-methoxyethyl)-1H-benzo[d][1,2,3]triazole (200.0 mg, 95.0%) as a brown solid. LCMS (ESI) m/z 256.0, [M+H]⁺.

### Step 4: N-(5-((1-(2-methoxyethyl)-1H-benzo[d][1,2,3]triazol-6-yl)ethynyl)-8-(methylamino)-2,7-naphthyridin-3-yl)cyclopropanecarboxamide

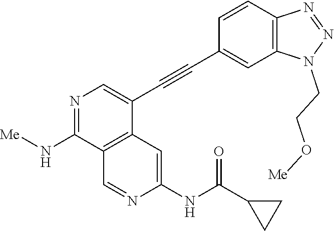 [SMILES: CNc1ncc(C#Cc2ccc3nnn(CCOC)c3c2)c2cc(NC(=O)C3CC3)ncc12]

N-(5-((1-(2-methoxyethyl)-1H-benzo[d][1,2,3]triazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-1-(2-methoxyethyl)-1H-benzo[d][1,2,3]triazole and as the starting material. LCMS (ESI) m/z 442.2, [M+H]⁺. ¹H NMR (300 MHz, DMSO-d₆) δ 11.11 (s, 1H), 9.39 (s, 1H), 8.77 (s, 1H), 8.43-8.36 (m, 1H), 8.33 (s, 1H), 8.17 (d, J=1.2 Hz, 1H), 8.10 (d, J=8.6 Hz, 1H), 7.54 (dd, J=8.6, 1.2 Hz, 1H), 4.93 (t, J=5.1 Hz, 2H), 3.88 (t, J=5.1 Hz, 2H), 3.24 (s, 3H), 3.04 (d, J=4.4 Hz, 3H), 2.18-2.06 (m, 1H), 0.98-0.81 (m, 4H).

### Example 683: Synthesis of N-(5-((1-ethyl-1H-benzo[d][1,2,3]triazol-6-yl)ethynyl)-8-(methylamino)-2,7-naphthyridin-3-yl)cyclopropanecarboxamide

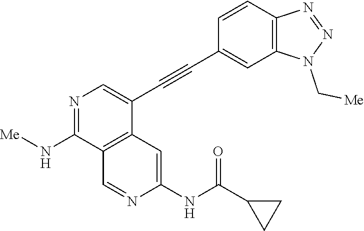 [SMILES: CCn1nnc2ccc(C#Cc3cnc(NC)c4cnc(NC(=O)C5CC5)cc34)cc21]

### Step 1: 5-bromo-N-ethyl-2-nitroaniline

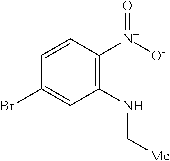 [SMILES: CCNc1cc(Br)ccc1[N+](=O)[O-]]

To a stirred solution of 4-bromo-2-fluoro-1-nitrobenzene (2.11 g; 9.092 mmol; 1.00 eq.) and ethylamine (814.1 mg; 18.051 mmol; 2.00 eq.) in DMF (10 mL) was added K₂CO₃ (3.78 g; 27.352 mmol; 3.00 eq.). The resulting mixture was stirred at room temperature for 12 hours under nitrogen atmosphere. The desired product was detected via LCMS. The resulting mixture was diluted with water (50 mL) and extracted with EtOAc (3×50 mL). The combined organic layers were washed with brine (3×20 mL) and dried over anhydrous Na₂SO₄. After filtration, the filtrate was concentrated under reduced pressure to afford 5-bromo-N-ethyl-2-nitroaniline as a yellow solid (2.16 g, 96.9%). LCMS (ESI) m/z 245.0, [M+H]⁺.

### Step 2: 5-bromo-N¹-ethylbenzene-1,2-diamine

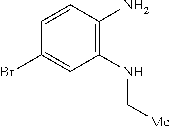 [SMILES: CCNc1cc(Br)ccc1N]

A solution of 5-bromo-N-ethyl-2-nitroaniline (1.10 g; 4.081 mmol; 1.00 eq.) and Fe (2.31 g; 41.182 mmol; 10.02 eq.) in AcOH. (1.5 mL) was stirred at 70° C. for 1 hour under nitrogen atmosphere. The desired product was detected via LCMS. The mixture was allowed to cool down to room temperature. The resulting mixture was diluted with water. (50 mL) and extracted with EtOAc (3×20 mL). The combined organic layers were washed with brine (3×20 mL) and dried over anhydrous Na₂SO₄. After filtration, the filtrate was concentrated under reduced pressure. The residue was purified by flash chromatography on silica gel column using 10-30% of EtOAc in petroleum ether as eluent to provide 5-bromo-N¹-ethylbenzene-1,2-diamine as a brown oil (580.2 mg, 66.0%). LCMS (ESI) m/z 215.0, [M+H]⁺.

### Step 3: 6-bromo-1-ethyl-1H benzo[d][1,2,3]triazole

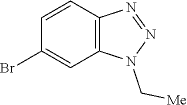 [SMILES: CCn1nnc2ccc(Br)cc21]

To a stirred solution of 5-bromo-N¹-ethylbenzene-1,2-diamine (200.1 mg; 0.930 mmol; 1.00 eq.) in HCl (4 M, 4 mL) was added dropwise a solution of NaNO₂ (110.2 mg; 1.594 mmol; 1.70 eq.) in water (1 mL) at 0° C. The resulting mixture was stirred at room temperature for 1 hour under nitrogen atmosphere. The desired product was detected via LCMS. The resulting mixture was diluted with water (50 mL) and neutralized to pH=7 with saturated NaHCO₃ solution. The aqueous phase was extracted with EtOAc (3×20 mL). The combined organic layers were washed with brine (3×20 mL) and dried over anhydrous Na₂SO₄. After filtration, the filtrate was concentrated under reduced pressure to afford 6-bromo-1-ethyl-1H-benzo[d][1,2,3]triazole as a yellow oil (180.1 mg, 85.6%). LCMS (ESI) m/z 226.0, [M+H]⁺.

### Step 4: N-(5-((1-ethyl-1H-benzo[d][1,2,3]triazol-6-yl)ethynyl)-8-(methylamino)-2,7-naphthyridin-3-yl)cydopropanecarboxamide

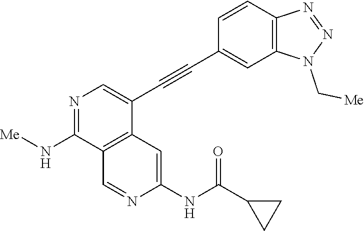 [SMILES: CCn1nnc2ccc(C#Cc3cnc(NC)c4cnc(NC(=O)C5CC5)cc34)cc21]

N-(5-((1-ethyl-1H-benzo[d][1,2,3]triazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-1-ethyl-1H-benzo[d][1,2,3]triazole as the starting material. LCMS (ESI) m/z 412.2, [M+H]⁺. ¹H NMR (400 MHz, DMSO-d₆) δ 11.1.6 (s, 1H), 9.40 (s, 1H), 8.81 (s, 1H), 8.44-8.38 (m, 1H), 8.33 (s, 1H), 8.22 (s, 1H), 8.10 (d, J=8.6 Hz, 1H), 7.53 (d, J=8.6 Hz, 1H), 4.85-4.73 (m, 2H), 3.04 (d, J=4.4 Hz, 3H), 2.16-2.06 (m, 1H), 1.56 (t, J=7.2 Hz, 3H), 0.95-0.85 (m, 4H).

### Example 684: Synthesis of N-(5-((1-(2-hydroxy-2-methylpropyl)-1H-benzo[d][1,2,3]triazol-6-yl)ethynyl)-8-(methylamino)-2,7-naphthyridin-3-yl)cyclopropanecarboxamide

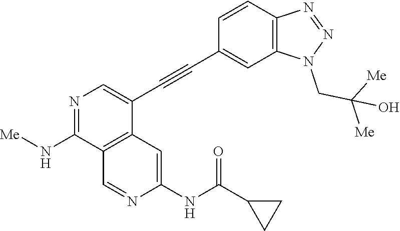 [SMILES: CNc1ncc(C#Cc2ccc3nnn(CC(C)(C)O)c3c2)c2cc(NC(=O)C3CC3)ncc12]

### Step 1: 1-((5-bromo-2-nitrophenyl)amino)-2-methylpropan-2-ol

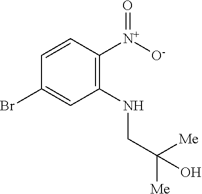 [SMILES: CC(C)(O)CNc1cc(Br)ccc1[N+](=O)[O-]]

To a stirred solution of 4-bromo-2-fluoro-1-nitrobenzene (2.10 g; 9.091 mmol; 1.00 eq.) and 1-amino-2-methylpropan-2-ol (894.2 mg; 10.021 mmol; 1.10 eq.) in DMF (10 mL) was added K₂CO₃ (2.52 g; 18.232 mmol; 2.00 eq.). The resulting mixture was stirred at room temperature for 12 hours under nitrogen atmosphere. The desired product was detected via LCMS. The mixture was diluted with water (50 mL) and extracted with EtOAc (3×20 mL). The combined organic layers were washed with brine (3×20 mL) and dried over anhydrous Na₂SO₄. After filtration, the filtrate was concentrated under reduced pressure to afford 1-((5-bromo-2-nitrophenyl)amino)-2-methylpropan-2-ol as a yellow solid (2.30 g, 87.5%). LCMS (ESI) m/z 289.0, [M+H]⁺.

### Step 2: 1-((2-amino-5-bromophenyl)amino)-2-methylpropan-2-ol

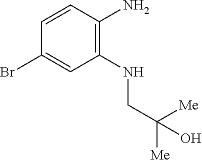 [SMILES: CC(C)(O)CNc1cc(Br)ccc1N]

To a stirred solution of 1-((5-bromo-2-nitrophenyl)amino)-2-methylpropan-2-ol (500.2 mg; 1.721 mmol; 1.00 eq.) in EtOH (10 mL) was added SnCl₂ (1.31 g; 6.883 mmol; 4.00 eq.). The resulting solution was stirred at 80° C. for 3 hours under nitrogen atmosphere. The desired product was detected via LCMS. The mixture was allowed to cool down to room temperature. The resulting mixture was concentrated under reduced pressure. The residue was purified by flash chromatography on silica gel column using 10-30% of EtOAc in petroleum ether as eluent to provide 1-((2-amino-5-bromophenyl)amino)-2-methylpropan-2-ol (400.2 mg, 89.2%). LCMS (ESI) m/z 259.0, [M+H]⁺.

### Step 3: 1-(6-bromo-1H-benzo[d][1,2,3]triazol-1-yl)-2-methylpropan-2-ol

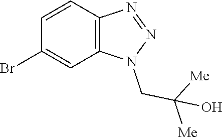 [SMILES: CC(C)(O)Cn1nnc2ccc(Br)cc21]

To a stirred solution of 1-((2-amino-5-bromophenyl)amino)-2-methylpropan-2-ol (200.3 mg; 0.772 mmol; 1.00 eq.) in HCl (12 M, 4 mL) was added a solution of NaNO₂ (90.5 mg; 1.312 mmol; 1.80 eq.) in water (1 mL) dropwise at 0° C. The resulting mixture was stirred at room temperature for 1 hour under nitrogen atmosphere. The desired product was detected via LCMS. The mixture was diluted with water (20 mL) and neutralized to pH=7 with saturated NaHCO₃ solution. The resulting mixture was extracted with EtOAc (3×20 mL). The combined organic layers were washed with brine (3×20 mL) and dried over anhydrous Na₂SO₄. After filtration, the filtrate was concentrated under reduced pressure to afford 1-(6-bromo-1H-benzo[d][1,2,3]triazol-1-yl)-2-methylpropan-2-ol as a brown oil (150.3 mg, 71.9%). LCMS (ESI) m/z 270.0, [M+H]⁺.

### Step 4: N-(5-((1-(2-hydroxy-2-methylpropyl)-1H-benzo[d][1,2,3]triazol-6-yl)ethynyl)-8-(methylamino)-2,7-naphthyridin-3-yl)cyclopropanecarboxamide

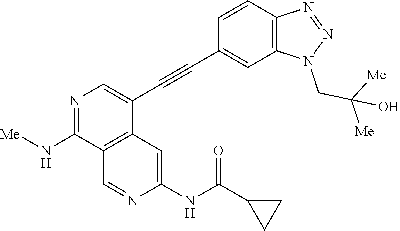 [SMILES: CNc1ncc(C#Cc2ccc3nnn(CC(C)(C)O)c3c2)c2cc(NC(=O)C3CC3)ncc12]

N-(5-((1-(2-hydroxy-2-methyl propyl)-1H-benzo[d][1,2,3]triazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 1-(6-bromo-1H-benzo[d][1,2,3]triazol-1-yl)-2-methylpropan-2-ol as the starting material. LCMS (ESI) m/z 456.2, [M+H]⁺. ¹H NMR (400 MHz, DMSO-d₆) δ 11.12 (s, 1H), 9.39 (s, 1H), 8.73 (s, 1H), 8.42-8.36 (m, 1H), 8.33 (s, 1H), 8.13 (d, J=1.2 Hz, 1H), 8.08 (d, J=8.6 Hz, 1H), 7.54-7.49 (m, 1H), 4.83 (s, 1H), 4.64 (s, 2H), 3.04 (d, J=4.4 Hz, 3H), 2.15-2.04 (m, 1H), 1.20 (s, 6H), 0.95-0.90 (m, 2H), 0.90-0.82 (m, 2H).

### Example 685: Synthesis of N-(5-((5-fluoro-1-methyl-1H-benzo[d][1,2,3]triazol-6-yl)ethynyl)-8-(methylamino)-2,7-naphthyridin-3-yl)cyclopropanecarboxamide

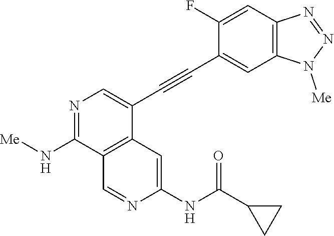 [SMILES: CNc1ncc(C#Cc2cc3c(cc2F)nnn3C)c2cc(NC(=O)C3CC3)ncc12]

### Step 1: 5-bromo-4-fluoro-N-methyl-2-nitroaniline

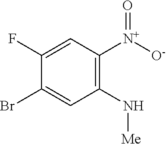 [SMILES: CNc1cc(Br)c(F)cc1[N+](=O)[O-]]

To a solution of 1-bromo-2,5-difluoro-4-nitrobenzene (2.00 g; 8.404 mmol; 1.00 eq.) and methylamine hydrochloride (0.60 g; 9.244 mmol; 1.10 eq.) in DMF (20 mL) was added K₂CO₃ (3.50 g; 25.212 mmol; 3.00 eq.) at room temperature. The resulting mixture was stirred at room temperature for 12 hours. The desired product was detected via LCMS. The resulting mixture was diluted with water (20 mL) and extracted with EtOAc (3×50 mL). The combined organic layers were washed with water (3×20 mL), dried over anhydrous Na₂SO₄. After filtration, the filtrate was concentrated under reduced pressure. The residue was purified by flash chromatography on silica gel column using 10-30% of EtOAc in petroleum ether as eluent to provide 5-bromo-4-fluoro-N-methyl-2-nitroaniline as a yellow solid (1.80 g, 86.1%). LCMS (ESI) m/z 249.0, [M+H]⁺.

### Step 2: 5-bromo-4-fluoro-M-methylbenzene-1,2-diamine

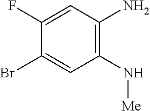 [SMILES: CNc1cc(Br)c(F)cc1N]

A solution of 5-bromo-4-fluoro-N-methyl-2-nitroaniline (200.0 mg; 0.803 mmol; 1.00 eq.) and Fe (448.4 mg; 8.030 mmol; 10.00 eq.) in AcOH (4 mL) was stirred at 70° C. for 1 hour under nitrogen atmosphere. The desired product was detected via LCMS. The mixture was allowed to cool down to room temperature. The resulting mixture was diluted with water (20 mL). The mixture was neutralized to pH=7 with saturated NaHCO₃ solution. The mixture was extracted with EtOAc (3×50 mL). The combined organic layers were washed with water (3×50 mL), dried over anhydrous Na₂SO₄. After filtration, the filtrate was concentrated under reduced pressure to 5-bromo-4-fluoro-N¹-methylbenzene-1,2-diamine as a brown oil (170.0 mg, 96.0%). LCMS (ESI) m/z 219.0, [M+H]⁺.

### Step 3: 6-bromo-5-fluoro-1-methyl-1H-benzo[d][1,2,3]triazole

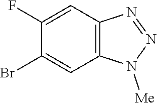 [SMILES: Cn1nnc2cc(F)c(Br)cc21]

To a stirred solution of 5-bromo-4-fluoro-N¹-methylbenzene-1,2-diamine (170.0 mg; 0.776 mmol; 1.00 eq.) in HCl (12 M, 4 mL) was added a solution of NaNO₂ (107.1 mg; 1.552 mmol; 2.00 eq.) in water (1 mL) at 0° C. The resulting solution was stirred at room temperature for 1 hour. The desired product was detected via LCMS. The mixture was diluted with water (20 mL). The mixture was neutralized to pH=7 with saturated NaHCO₃ solution and extracted with EtOAc (3×50 mL). The combined organic layers were washed with water (3×50 mL), dried over anhydrous Na₂SO₄. The solvent was concentrated under reduced pressure to afford 6-bromo-5-fluoro-1-methyl-1/1-benzo[d][1,2,3]triazole as an off-white solid (150.0 mg, 84.0%). LCMS (ESI) m/z 230.0, [M+H]⁺.

### Step 4: N-(5-((5-fluoro-1-methyl-1H-benzo[d][1,2,3]triazol-6-yl)ethynyl)-8-(methylamino)-2,7-naphthyridin-3-yl)cyclopropanecarboxamide

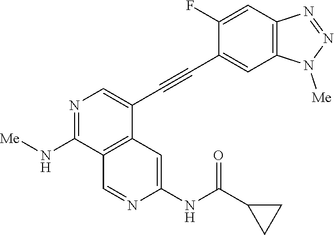 [SMILES: CNc1ncc(C#Cc2cc3c(cc2F)nnn3C)c2cc(NC(=O)C3CC3)ncc12]

N-(5-((5-fluoro-1-methyl-1H-benzo[d][1,2,3]triazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-5-fluoro-1-methyl-1H-benzo[d][1,2,3]triazole and as the starting material. LCMS (ESI) m/z 416.2, [M+H]⁺. ¹H NMR (400 MHz, DMSO-d₆) δ 11.12 (s, 1H), 9.39 (s, 1H), 8.72 (s, 1H), 8.49-8.41 (m, 1H), 8.34 (s, 1H), 8.28 (d, J=6.0 Hz, 1H), 8.04 (d, J=9.1 Hz, 1H), 4.35 (s, 3H), 3.04 (d, J=4.4 Hz, 3H), 2.14-2.06 (m, 1H), 0.93-0.81 (m, 4H).

### Example 686: Synthesis of N-(5-((1-(2-cyanoethyl)-1H-benzo[d][1,2,3]triazol-6-yl)ethynyl)-8-(methylamino)-2,7-naphthyridin-3-yl)cyclopropanecarboxamide

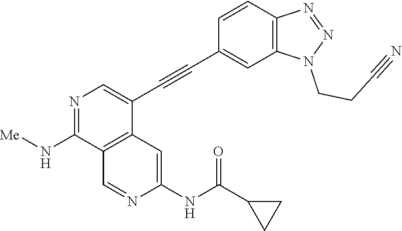 [SMILES: CNc1ncc(C#Cc2ccc3nnn(CCC#N)c3c2)c2cc(NC(=O)C3CC3)ncc12]

### Step 1: 3-((5-bromo-2-nitrophenyl)amino)propanenitrile

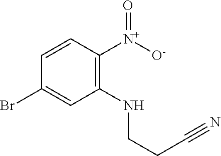 [SMILES: N#CCCNc1cc(Br)ccc1[N+](=O)[O-]]

To a stirred solution of 4-bromo-2-fluoro-1-nitrobenzene (2.00 g; 8.404 mmol; 1.00 eq.) and 3-aminopropanenitrile (0.60 g; 9.244 mmol; 1.10 eq.) in DMF (10 mL) was added K₂CO₃ (3.50 g; 25.212 mmol; 3.00 eq.) at room temperature. The resulting mixture was stirred at room temperature for 12 hours. The desired product was detected via LCMS. The resulting mixture was diluted with water (20 mL) and extracted with EtOAc (3×50 mL). The combined organic layers were washed with water (3×50 mL), dried over anhydrous Na₂SO₄. After filtration, the filtrate was concentrated under reduced pressure. The residue was purified by flash chromatography on silica gel column using 10-30% of EtOAc in petroleum ether as eluent to provide 3-((5-bromo-2-nitrophenyl)amino)propanenitrile (1.80 g, 86.0%) as a yellow solid. LCMS (ESI) m/z 270.0, [M+H]⁺.

### Step 2: 3-((2-amino-5-bromophenyl)amino)propanenitrile

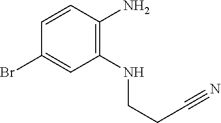 [SMILES: N#CCCNc1cc(Br)ccc1N]

A solution of 3-((5-bromo-2-nitrophenyl)amino)propanenitrile (200.0 mg; 0.803 mmol; 1.00 eq.) and Fe (448.5 mg; 8.030 mmol; 1.0.00 eq.) in AcOH (4 mL) was stirred at 70° C. for 1 hour under nitrogen atmosphere. The desired product was detected via LCMS. The mixture was allowed to cool down to room temperature. The resulting mixture was diluted with water (20 mL) and neutralized to pH=7 with saturated NaHCO₃ solution. The mixture was extracted with EtOAc (3×50 mL). The combined organic layers were washed with water (3×50 mL), dried over anhydrous Na₂SO₄. After filtration, the filtrate was concentrated under reduced pressure to afford 3-((2-amino-5-bromophenyl)amino)propanenitrile (170.0 mg, 96.0%) as a brown oil. LCMS (ESI) m/z 240.0, [M+H]⁺.

### Step 3: 3-(6-bromo-1H-benzo[d][1,2,3]triazol-1-yl)propanenitrile

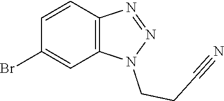 [SMILES: N#CCCn1nnc2ccc(Br)cc21]

To a stirred solution of 3-((2-amino-5-bromophenyl)amino)propanenitrile (170.0 mg; 0.776 mmol; 1.00 eq.) in HCl (4 M, 4 mL) was added a solution of NaNO₂ (107.1 mg; 1.552 mmol; 2.00 eq.) in water (1 mL) at 0° C. The resulting solution was stirred at room temperature for 1 hour. The desired product was detected via LCMS. The resulting mixture was diluted with water (10 mL) and neutralized to pH=7 with saturated NaHCO₃ solution. The mixture was extracted with EtOAc (3×20 mL). The combined organic layers were washed with water (3×20 mL), dried over anhydrous Na₂SO₄. After filtration, the filtrate was concentrated under reduced pressure to afford 3-(6-bromo-1H-benzo[d][1,2,3]triazol-1-yl)propanenitrile (150.0 mg, 84.0%) as an off-white solid. LCMS (ESI) m/z 251.0, [M+H]⁺.

### Step 4: N-(5-((1-(2-cyanoethyl)-1H-benzo[d][1,2,3]triazol-6-yl)ethynyl)-8-(methylanilino)-2,7-naphthyridin-3-yl)cyclopropanecarboxamide

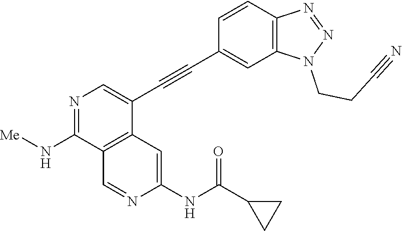 [SMILES: CNc1ncc(C#Cc2ccc3nnn(CCC#N)c3c2)c2cc(NC(=O)C3CC3)ncc12]

N-(5-((1-(2-cyanoethyl)-1H-benzo[d][1,2,3]triazol-6-yl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 3-(6-bromo-1H-benzo[d][1,2,3]triazol-1-yl)propanenitrile as the starting material. LCMS (ESI) m/z 437.2, [M+H]⁺. ¹H NMR (400 MHz, DMSO-d₆) δ 11.14 (s, 1H), 9.40 (s, 1H), 8.74 (s, 1H), 8.45-8.40 (m, 1H), 8.33 (s, 1H), 8.28 (s, 1H), 8.15 (d, J=8.6 Hz, 1H), 7.58 (dd, J=8.6, 1.4 Hz, 1H), 5.07 (t, J=6.4 Hz, 2H), 3.30 (t, J=6.4 Hz, 2H), 3.04 (d, J=4.4 Hz, 3H), 2.14-2.05 (m, 1H), 0.97-0.89 (m, 2H), 0.89-0.82 (m, 2H).

### Example 687: Synthesis of N-(5-((1-cyclopropyl-1H-benzo[d][1,2,3]triazol-6-yl)ethynyl)-8-(methylimino)-2,7-naphthyridin-3-yl)cyclopropanecarboxamide

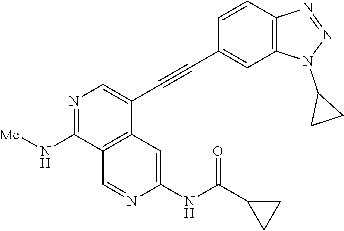 [SMILES: CNc1ncc(C#Cc2ccc3nnn(C4CC4)c3c2)c2cc(NC(=O)C3CC3)ncc12]

### Step 1: 5-bromo-N-cyclopropyl-2-nitroaniline

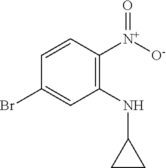 [SMILES: O=[N+]([O-])c1ccc(Br)cc1NC1CC1]

To a stirred solution of 4-bromo-2-fluoro-1-nitrobenzene (3.01 g; 13.632 mmol; 1.00 eq.) and cyclopropanamine (860.2 mg; 15.061 mmol; 1.10 eq.) in DMF (30 mL) was added K₂CO₃ (3.78 g; 27.351 mmol; 2.00 eq.). The resulting mixture was stirred at room temperature for 12 hours under nitrogen atmosphere. The desired product was detected via LCMS. The mixture was diluted with water (50 mL) and extracted with EtOAc (3×30 mL). The combined organic layers were washed with brine (3×30 mL) and dried over anhydrous Na₂SO₄. After filtration, the filtrate was concentrated under reduced pressure to afford 5-bromo-N-cyclopropyl-2-nitroaniline as a yellow oil (3.41 g, 96.9%). LCMS (ESI) m/z 257.0, [M+H]⁺.

### Step 2: 5-bromo-N¹-cyclopropylbenzene-1,2-diamine

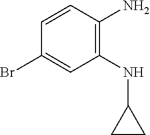 [SMILES: Nc1ccc(Br)cc1NC1CC1]

To a stirred solution of 5-bromo-N-cyclopropyl-2-nitroaniline (200.2 mg; 0.778 mmol; 1.00 eq.) and Fe (218.2 mg; 3.904 mmol, 5.02 eq.) in a mixture solvent of EtOH/water (5:1, 2 mL) was added NH₄Cl (208.3 mg; 3.881 mmol; 5.00 eq.). The resulting mixture was stirred at 80° C. for 1 hour under nitrogen atmosphere. The desired product was detected via LCMS. The mixture was allowed to cool down to room temperature. The reaction was diluted with water (50 mL) and extracted with EtOAc (3×20 mL). The combined organic layers were washed with brine (3×20 mL) and dried over anhydrous Na₂SO₄. After filtration, the filtrate was concentrated under reduced pressure to afford 5-bromo-N¹-cyclopropylbenzene-1,2-diamine as a brown oil (160.1 mg, 90.5%). LCMS (ESI) m/z 227.0, [M+H]⁺.

### Step 3: 6-bromo-1-cyclopropyl-1H-benzo[d][1,2,3]triazole

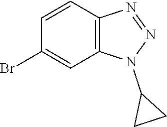 [SMILES: Brc1ccc2nnn(C3CC3)c2c1]

To a stirred solution of 5-bromo-N¹-cyclopropylbenzene-1,2-diamine (160.0 mg; 0.791 mmol; 1.00 eq.) in HCl (12 M, 4 mL) was added a solution of NaNO₂ (60.1 mg; 0.871 mmol; 1.80 eq.) in water (1 mL) dropwise at 0° C. The resulting mixture was stirred at room temperature for 1 hour under nitrogen atmosphere. The desired product was detected via LCMS. The reaction was diluted with water (50 mL) and neutralized to pH=7 with saturated Na₂CO₃ solution. The resulting mixture was extracted with EtOAc (3×20 mL). The combined organic layers was washed with brine (3×20 mL) and dried over anhydrous Na₂SO₄. After filtration, the filtrate was concentrated under reduced pressure. The residue was purified by flash chromatography on silica gel column using 10-20% of EtOAc in petroleum ether as eluent to provide 6-bromo-1-cyclopropyl-1H-benzo[d][1,2,3]triazole as a white solid (60.3 mg, 31.8%). LCMS (ESI) m/z 238.0, [M+H]⁺.

### Step 4: N-(5-((1-cyclopropyl-1H-benzo[d][1,2,3]triazol-6-yl)ethynyl)-8-(methylamino)-2,7-naphthyridin-3-yl)cydopropanecarboxamide

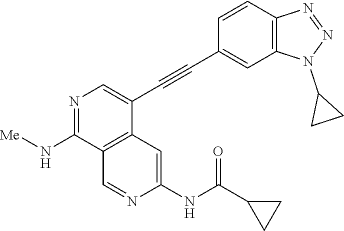 [SMILES: CNc1ncc(C#Cc2ccc3nnn(C4CC4)c3c2)c2cc(NC(=O)C3CC3)ncc12]

N-(5-((1-cyclopropyl-1H-benzo[d][1,2,3]triazol-6-yl)ethynyl)-8-(methylamino)-2,7-naphthyridin-3-yl)cyclopropanecarboxamide was synthesized using a similar procedure that was previously described in Example 595 by using N-(5-ethynyl-8-(methylamino)-2,7-naphthyridin-3-yl)cyclopropanecarboxamide and 6-bromo-1-cyclopropyl-1H-benzo[d][1,2,3]triazole as the starting material. LCMS (ESI) m/z 424.2, [M+H]⁺. ¹H NMR (400 MHz, DMSO-d₆) δ 11.13 (s, 1H), 9.37 (s, 1H), 8.79 (s, 1H), 8.45-8.38 (m, 1H), 8.35 (s, 1H), 8.14-8.06 (m, 2H), 7.55 (dd, J=8.6, 1.4 Hz, 1H), 4.10-4.00 (m, 1H), 3.04 (d, J=4.4 Hz, 3H), 2.16-2.05 (m, 1H), 1.36-1.26 (m, 4H), 0.93-0.83 (m, 4H).

### Example 688: Synthesis of N-(5-((1-(3-methoxypropyl)-1H-benzo[d][1,2,3]triazol-6-yl)ethynyl)-8-(methylamino)-2,7-naphthyridin-3-yl)cyclopropanecarboxamide

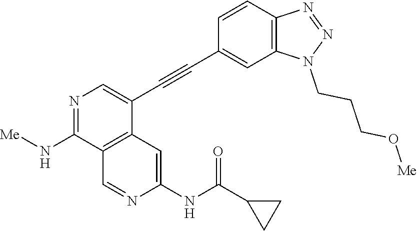 [SMILES: CNc1ncc(C#Cc2ccc3nnn(CCCOC)c3c2)c2cc(NC(=O)C3CC3)ncc12]

### Step 1: 5-bromo-N-(3-methoxypropyl)-2-nitroaniline

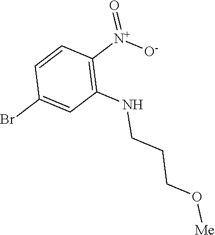 [SMILES: COCCCNc1cc(Br)ccc1[N+](=O)[O-]]

To a stirred solution of 4-bromo-2-fluoro-1-nitrobenzene (2.01 g; 9.091 mmol; 1.00 eq.) and 3-methoxypropylamine (894.5 mg; 10.021 mmol; 1.10 eq.) in DMF (20 mL) was added K₂CO₃ (2.51 g; 18.085 mmol; 2.00 eq.). The resulting mixture was stirred at room temperature for 12 hours under nitrogen atmosphere. The desired product was detected via LCMS. The resulting mixture was diluted with water (50 mL) and extracted with EtOAc (3×20 mL). The combined organic layers were washed with brine (3×20 mL) and dried over anhydrous Na₂SO₄. After filtration, the filtrate was concentrated under reduced pressure to afford 5-bromo-N-(3-methoxypropyl)-2-nitroaniline as a yellow solid (2.50 g, 95.1%). LCMS (ESI) m/z 289.0, [M+H]⁺.

### Step 2: 5-bromo-N¹-(3-methoxypropyl)benzene-1,2-diamine

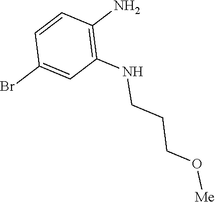 [SMILES: COCCCNc1cc(Br)ccc1N]

A solution of 5-bromo-N-(3-methoxypropyl)-2-nitroaniline (1.01 g; 3.445 mmol; 1.00 eq.) and Fe (1.91 g; 34.020 mmol; 10.0 eq.) in AcOH (15 mL) was stirred at 70° C. for 2 hours under nitrogen atmosphere. The desired product was detected via LCMS. The mixture was allowed to cool down to room temperature. The resulting mixture was diluted with water (50 mL) and neutralized to pH=7 with saturated NaHCO₃ solution. The resulting mixture was extracted with EtOAc (3×20 mL). The combined organic layers were washed with brine (3×20 mL) and dried over anhydrous Na₂SO₄. After filtration, the filtrate was concentrated under reduced pressure. The residue was purified by flash chromatography on silica gel column using 10-20% of EtOAc in petroleum ether as eluent to provide 5-bromo-N¹-(3-methoxypropyl))benzene-1,2-diamine as a black oil (800.3 mg, 89.2%). LCMS (ESI) m/z 259.0, [M+H]⁺.

### Step 3: 6-bromo-1-(3-methoxypropyl)-1N-benzo[d][1,2,3]triazole

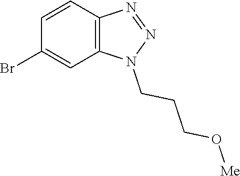 [SMILES: COCCCn1nnc2ccc(Br)cc21]

To a stirred solution of 5-bromo-N¹-(3-methoxypropyl)benzene-1,2-diamine (200.0 mg; 0.772 mmol; 1.00 eq.) in HCl (12 M, 4 mL) was added a solution of NaNO₂ (58.5 mg; 0.849 mmol; 1.10 eq.) in water (1 mL) dropwise at 0° C. The resulting mixture was stirred at room temperature for 1 hour under nitrogen atmosphere. The desired product was detected via LCMS. The resulting mixture was diluted with water (50 mL) and neutralized to pH==7 with saturated NaHCO₃ solution. The resulting mixture was extracted with EtOAc (3×20 mL). The combined organic layers were washed with brine (3×20 mL) and dried over anhydrous Na₂SO₄. After filtration, the filtrate was concentrated under reduced pressure. The residue was purified by flash chromatography on silica gel column using 10-15% of EtOAc in petroleum ether as eluent to provide 6-bromo-1-(3-methoxypropyl)-1H-benzo[d][1,2,3]triazole as a brown oil (90.3 mg, 43.1%). LCMS (ESI) m/z 270.1, [M+H]⁺.

### Step 4: N-(5-((1-(3-methoxypropyl)-1H-benzo[d][1,2,3]triazol-6-yl)ethynyl)-8-(methylamino)-2,7-naphthyridin-3-yl)cyclopropanecarboxamide

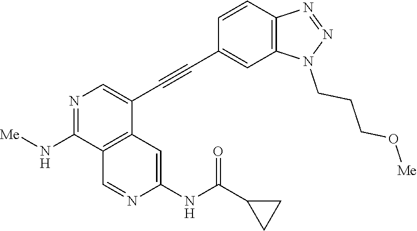 [SMILES: CNc1ncc(C#Cc2ccc3nnn(CCCOC)c3c2)c2cc(NC(=O)C3CC3)ncc12]

N-(5-((1-(3-methoxypropyl)-1H-benzo[d][1,2,3]triazol-6-yl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 6-bromo-1-(3-methoxypropyl)-1H-benzo[d][1,2,3]triazole as the starting material. LCMS (ESI) m/z 456.2, [M+H]⁺. ¹H NMR (400 MHz, DMSO-d₆) δ 11.13 (s, 1H), 9.39 (s, 1H), 8.77 (s, 1H), 8.43-8.31 (m, 2H), 8.16-8.07 (m, 2H), 7.54 (dd, J=8.6, 1.2 Hz, 1H), 4.79 (t, J=6.8 Hz, 2H), 3.33 (t, J=6.8 Hz, 2H), 3.22 (s, 3H), 3.04 (d, J=4.4 Hz, 3H), 2.20-2.06 (m, 2H), 2.14-2.05 (m, 1H), 0.96-0.82 (m, 4H).

### Example 689: Synthesis of N-(5-((1-methyl-1H-[1,2,3]triazolo[4,5-c]pyridin-6-yl)ethynyl)-8-(methylamino)-2,7-naphthyridin-3-yl)cyclopropanecarboxamide

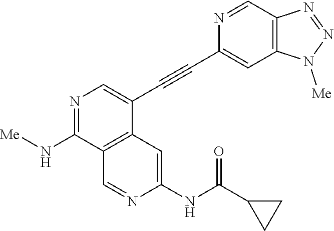 [SMILES: CNc1ncc(C#Cc2cc3c(cn2)nnn3C)c2cc(NC(=O)C3CC3)ncc12]

### Step 1: 2-bromo-N-methyl-5-nitropyridin-4-amine

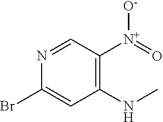

To a solution of 2,4-dibromo-5-nitropyridine (1.00 g; 3.547 mmol; 1.00 eq.) in THF (20 mL) was added methylamine (2 M in THF, 3.6 mL) at 0° C. The resulting solution was stirred at room temperature for 12 hours. The desired product was detected via LCMS. The solvent was concentrated under vacuum. The residue was purified by flash chromatography on silica gel column using 0-50% of EtOAc in petroleum ether as eluent to provide 2-bromo-N-methyl-5-nitropyridin-4-amine as a yellow solid (460.0 mg, 53.1%). LCMS (ESI) m/z 231.9, [M+H]⁺.

### Step 2: 6-bromo-N⁴-methylpyridine-3,4-diamine

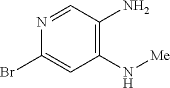

A solution of 2-bromo-N-methyl-5-nitropyridin-4-amine (460.0 mg; 1.982 mmol; 1.00 eq.) and Fe (442.8 mg; 7.928 mmol; 4.00 eq.) in AcOH (14 mL) was stirred at 70° C. for 3 hours. The desired product was detected via LCMS. The resulting mixture was filtered, the filtrate was neutralized to pH=7 with saturated K₂CO₃ solution. The mixture was extracted with EtOAc (3×50 mL). The combined organic layers were washed with brine (3×50 mL), dried over anhydrous Na₂SO₄. After filtration, the filtrate was concentrated under reduced pressure. The residue was purified by flash chromatography on silica gel column using 0-50% of EtOAc in petroleum ether as eluent to provide 6-bromo-N⁴-methylpyridine-3,4-diamine as a yellow solid (320.0 mg, 77.5%). LCMS (ESI) m/z 202.1, [M+H]⁺.

### Step 3: 6-bromo-1-methyl-1H-[1,2,3]triazolo[4,5-c]pyridine

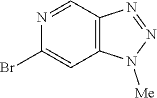 [SMILES: Cn1nnc2cnc(Br)cc21]

To a solution of 6-bromo N⁴-methylpyridine-3,4-diamine (300.0 mg; 1.485 mmol; 1.00 eq.) in HCl (12 M, 4 mL) was added a solution of NaNO₂ (121.9 mg; 1.767 mmol; 1.20 eq.) in water (1 mL) at 0° C. The resulting mixture was stirred at room temperature for 1 hour under nitrogen atmosphere. The desired product was detected via LCMS. The mixture was neutralized to pH=7 with saturated NaHCO₃ solution and extracted with EtOAc (3×50 mL). The combined organic layers were washed with brine (3×20 mL), dried over anhydrous Na₂SO₄. After filtration, the filtrate was concentrated under reduced pressure. The residue was purified by flash chromatography on silica gel column using 0-50% of EtOAc in petroleum ether as eluent to provide 6-bromo-1-methyl-1H-[1,2,3]triazolo[4,5-c]pyridine as a yellow solid (300.0 mg, 90.1%). LCMS (ESI) m/z 212.9, [M+H]⁺.

### Step 4: N-(5-((1-methyl-1H-[1,2,3]triazolo[4,5-c]pyridin-6-yl)ethynyl)-8-(methylamino)-2,7-naphthyridin-3-yl)cyclopropanecarboxamide

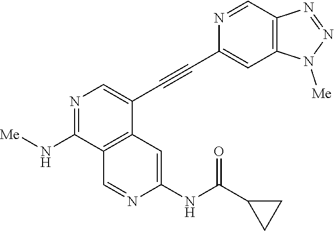 [SMILES: CNc1ncc(C#Cc2cc3c(cn2)nnn3C)c2cc(NC(=O)C3CC3)ncc12]

N-(5-((1-methyl-1H-[1,2,3]triazolo[4,5-c]pyridin-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-1-methyl-1H-[1,2,3]triazolo[4,5-c]pyridine as the starting material. LCMS (ESI) m/z 399.1, [M+H]⁺. ¹H NMR (400 MHz, DMSO-d₆) δ 11.14 (s, 1H), 9.60 (s, 1H), 9.46 (s, 1H), 8.73 (s, 1H), 8.50-8.41 (m, 1H), 8.38 (s, 1H), 8.27 (s, 1H), 4.37 (s, 3H), 3.04 (d, J=4.4 Hz, 3H), 2.15-2.08 (m, 1H), 0.94-0.85 (m, 4H).

### Example 690: Synthesis of (R)—N-(8-(methylamino)-5-((4-((tetrahydrofuran-3-yl)oxy)phenyl)ethynyl)-2,7-naphthyridin-3-yl)cyclopropanecarboxamide

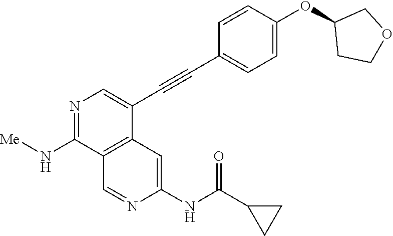 [SMILES: CNc1ncc(C#Cc2ccc(O[C@@H]3CCOC3)cc2)c2cc(NC(=O)C3CC3)ncc12]

### Step 1: (5)-tetrahydrofuran-3-yl methanesulfonate

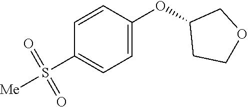 [SMILES: CS(=O)(=O)c1ccc(O[C@H]2CCOC2)cc1]

To a stirred solution of (S)-tetrahydrofuran-3-ol (500.0 mg; 5.675 mmol; 1.00 eq.) and DIEA (733.5 mg; 5.675 mmol; 1.00 eq.) in CH₂Cl₂ (1.0 mL) was added methanesulfonic anhydride (1.98 g; 11.350 mmol; 2.00 eq.) at 0° C. under nitrogen atmosphere. The resulting mixture was stirred at room temperature for 14 hours. The resulting mixture was quenched with saturated NaHCO₃ solution (10 mL) and extracted with EtOAc (3×35 mL). The organic layer was dried over anhydrous Na₂SO₄. After filtration, the filtrate was concentrated under reduced pressure to afford (S)-tetrahydrofuran-3-yl methanesulfonate as a brown oil (499.5 mg, crude). The crude product was used in the next step directly without further purification.

### Step 2: (R)-3-(4-bromophenoxy)tetrahydrofuran

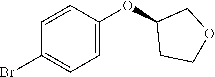 [SMILES: Brc1ccc(O[C@@H]2CCOC2)cc1]

A solution of 4-bromophenol (260.0 mg; 1.503 mmol; 1.00 eq.), (S)-tetrahydrofuran-3-yl methanesulfonate (499.5 mg; 3.006 mmol; 2.00 eq.) and Cs₂CO₃ (1.47 g; 4.508 mmol; 3.00 eq.) in DMF (5 mL) was stirred at 100° C. for 14 hours under nitrogen atmosphere. The mixture was diluent with water (20 mL) and extracted with EtOAc (3×30 mL). The combined organic layers were washed with brine (5×10 mL) and dried over anhydrous Na₂SO₄. After filtration, the filtrate was concentrated under reduced pressure. The residue was purified by flash chromatography on silica gel column using 10-30% of EtOAc in petroleum ether as eluent to provide (R)-3-(4-bromophenoxy)tetrahydrofuran as a light yellow oil (270.0 mg, 71.7%). 4H NMR (400 MHz, DMSO-d₆) δ 7.53-7.37 (m, 2H), 7.01-6.81 (m, 2H), 5.09-4.95 (m, 1H), 3.97-3.66 (m, 4H), 2.31-2.13 (m, 1H), 2.02-1.83 (m, 1H).

### Step 3: (R)—N-(8-(methylamino)-5-((4-((tetrahydrofuran-3-yl)oxy)phenyl)ethynyl)-2,7-naphthyridin-3-yl)cyclopropanecarboxamide

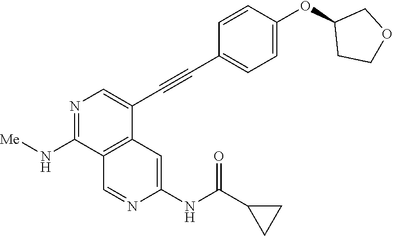 [SMILES: CNc1ncc(C#Cc2ccc(O[C@@H]3CCOC3)cc2)c2cc(NC(=O)C3CC3)ncc12]

(R)—N-(8-(methylamino)-5-((4-((tetrahydrofuran-3-yl)oxy)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and (R)-3-(4-bromophenoxy)tetrahydrofuran as the starting material. LCMS (ESI) m/z 429.2, [M+H]⁺. ¹H NMR (400 MHz, DMSO-d₆) δ 11.07 (s, 1H), 9.37 (s, 1H), 8.64 (s, 1H), 8.32-8.26 (m, 1H), 8.25 (s, 1H), 7.52-7.47 (m, 2H), 7.07-6.98 (m, 2H), 5.12-5.07 (m, 1H), 3.95-3.74 (m, 4H), 3.02 (d, J=4.4 Hz, 3H), 2.33-2.21 (m, 1H), 2.12-2.04 (m, 1H), 2.04-1.94 (m, 1H), 0.91-0.83 (m, 4H).

### Example 691: Synthesis of (S)—N-(8-(methylamino)-5-((4-((tetrahydrofuran-3-yl)oxy)phenyl)ethynyl)-2,7-naphthyridin-3-yl)cyclopropanecarboxamide

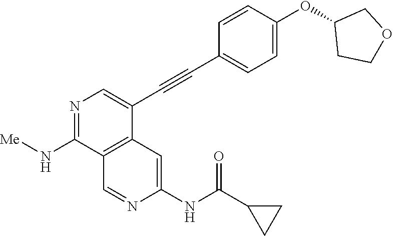 [SMILES: CNc1ncc(C#Cc2ccc(O[C@H]3CCOC3)cc2)c2cc(NC(=O)C3CC3)ncc12]

### Step 1: (R)-tetrahydrofuran-3-yl methanesulfonate

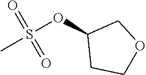

A solution of (R)-tetrahydrofuran-3-ol (500.0 mg; 5.675 mmol; 1.00 eq.), methanesulfonic anhydride (1977.0 mg; 11.350 mmol; 2.00 eq.) and DIEA (733.0 mg; 5.671 mmol; 1.00 eq.) in CH₂Cl₂ (10 mL) was stirred at room temperature for 2 hours under nitrogen atmosphere. The resulting mixture was quenched with NaHCO₃ aqueous solution (20 mL) and extracted with EtOAc (3×50 mL). The combined organic layers were washed with brine (3×25 mL) and dried over anhydrous Na₂SO₄. After filtration, the filtrate was concentrated under reduced pressure to afford (R)-tetrahydrofuran-3-yl methanesulfonate as a brown oil (916 mg, crude). ¹H NMR (400 MHz, DMSO-d₆) δ 5.36-5.25 (m, 1H), 3.91-3.69 (m, 4H), 3.22 (s, 3H), 2.28-2.02 (m, 2H).

### Step 2: (S)-3-(4-bromophenoxy)tetrahydrofuran

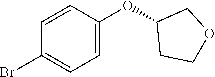 [SMILES: Brc1ccc(O[C@H]2CCOC2)cc1]

A solution of (R)-tetrahydrofuran-3-yl methanesulfonate (499.5 mg; 3.006 mmol; 2.00 eq.), 4-bromophenol (260.0 mg; 1.503 mmol; 1.00 eq.) and Cs₂CO₃ (1468.9 mg; 4.508 mmol; 3.00 eq.) in DMF (5 mL) was stirred at 100° C. for 14 hours under nitrogen atmosphere. The desired product was detected via LCMS. The solution was diluted with water (10 mL) and extracted with EtOAc (3×50 mL). The combined organic layers were washed with brine (3×25 mL) and dried over anhydrous Na₂SO₄. After filtration, the filtrate was concentrated under reduced pressure. The residue was purified by flash chromatography on silica gel column using 10-30% of EtOAc in petroleum ether as eluent to provide (S)-3-(4-bromophenoxy)tetrahydrofuran as a white solid (304.0 mg, 83.2%). LCMS (ESI) m/z 243.2, [M+H]⁺.

### Step 3: (S)—N-(8-(methylamino)-5-((4-((tetrahydrofuran-3-yl)oxy)phenyl)ethynyl)-2,7-naphthyridin-3-yl)cyclopropanecarboxamide

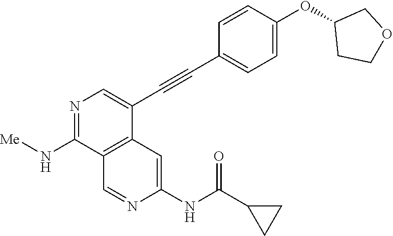 [SMILES: CNc1ncc(C#Cc2ccc(O[C@H]3CCOC3)cc2)c2cc(NC(=O)C3CC3)ncc12]

(S)—N-(8-(methyl amino)-5-((4-((tetrahydrofuran-3-yl)oxy)phenyl)ethynyl)-2,7-naphthyridin-3-yl)cyclopropanecarboxamide as synthesized using a similar procedure that was previously described in Example 595, by using N-(5-ethynyl-8-(methylamino)-2,7-naphthyridin-3-yl)cyclopropanecarboxamide and (S)-3-(4-bromophenoxy)tetrahydrofuran as the starting material. LCMS (ESI) m/z 429.2, [M+H]⁺. ¹H NMR (400 MHz, DMSO-d₆) δ 11.07 (s, 1H), 9.37 (s, 1H), 8.64 (s, 1H), 8.32-8.26 (m, 1H), 8.25 (s, 1H), 7.53-7.48 (m, 2H), 7.05-6.98 (m, 2H), 5.13-5.05 (m, 1H), 3.95-3.72 (m, 4H), 3.02 (d, J=4.4 Hz, 3H), 2.32-2.1.5 (m, 1H), 2.13-2.02 (m, 1H), 2.02-1.92 (m, 1H), 0.93-0.79 (m, 4H).

### Example 692: Synthesis of N-(5-((2-(methoxymethyl)-1-methyl-1H-benzoimidazol-6-yl)ethynyl)-8-(methylamino)-2,7-naphthyridin-3-yl)cyclopropanecarboxamide

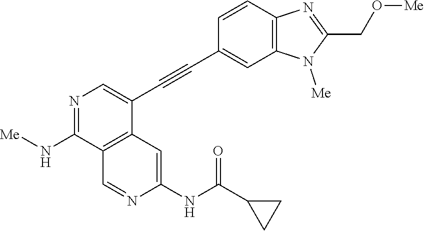 [SMILES: CNc1ncc(C#Cc2ccc3nc(COC)n(C)c3c2)c2cc(NC(=O)C3CC3)ncc12]

### Step 1: (6-bromo-1-methyl-1H-benzo[d]imidazol-2-yl)methanol

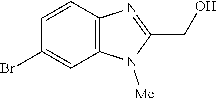 [SMILES: Cn1c(CO)nc2ccc(Br)cc21]

A solution of 5-bromo-N¹-methylbenzene-1,2-diamine (500.0 mg; 2.487 mmol; 1.00 eq.) and 2-hydroxyacetic acid (945.5 mg; 12.435 mmol; 5.00 eq.) in HCl (3 M, 5 mL) was stirred at 100° C. for 2 hours. The desired product was detected via LCMS. The mixture was basified to pH=8 with saturated NaHCO₃ solution. The mixture was filtered, the filter cake was collected to provide (6-bromo-1-methyl-1H-benzo[d]imidazol-2-yl)methanol as a brown solid (521.0 mg, 85.7%). LCMS (ESI) m/z 241.0, [M+H]⁺.

### Step 2: 6-bromo-2-(methoxymethyl)-1-methyl-1H-benzo[d]imidazole

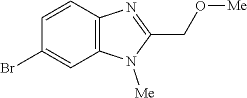 [SMILES: COCc1nc2ccc(Br)cc2n1C]

To a stirred solution of (6-bromo-1-methyl-1H-benzo[d]imidazol-2-yl)methanol (420.0 mg; 1.742 mmol; 1.00 eq.) in THF (20.0 mL) was added NaH (60% dispersion in mineral oil, 209.0 mg; 5.226 mmol; 3.00 eq.) at 0° C. After stirring for 10 minutes to the above solution was added MeI (247.2 mg; 1.742 mmol; 1.00 eq.). The resulting solution was stirred at room temperature for 3 hours under nitrogen atmosphere. The desired product was detected via LCMS. The reaction was quenched by the addition of water (10 mL) and extracted with EtOAc (1×50 mL). The organic layer was washed with brine (3×5 mL) and concentrated under vacuum. The residue was purified by flash chromatography on silica gel column using 0-10% of EtOAc in petroleum ether as eluent to provide 6-bromo-2-(methoxymethyl)-1-methyl-1H-benzo[d]imidazole as a brown oil (171.6 mg, 37.0%). LCMS (ESI) m/z 255.0, [M+H]⁺. 1.5 Step 3: N-(5-((2-(methoxymethyl)-1-methyl-1H-benzo[d]imidazol-6-yl)ethynyl)-8-(methylamino)-2,7-naphthyridin-3-yl)cyclopropanecarboxamide

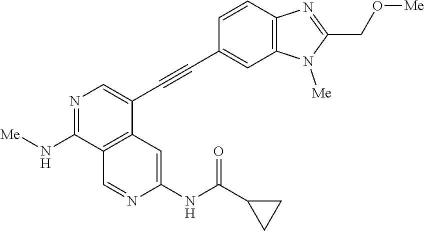 [SMILES: CNc1ncc(C#Cc2ccc3nc(COC)n(C)c3c2)c2cc(NC(=O)C3CC3)ncc12]

N-(5-((2-(methoxymethyl)-1-methyl-1H-benzo[d]imidazol-6-yl)ethynyl)-8-(methyl amino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-2-(methoxymethyl)-1-methyl-1H-benzo[d]imidazole as the starting material. LCMS (ESI) m/z 441.2, [M+H]⁺. ¹H NMR (400 MHz, DMSO-d₆) δ 11.11 (s, 1H), 9.39 (s, 1H), 8.80 (s, 1H), 8.32-8.29 (m, 1H), 8.28 (s, 1H), 7.92 (d, J=2.0 Hz, 1H), 7.66 (d, J=8.4 Hz, 1H), 7.41-7.36 (m, 1H), 4.73 (s, 2H), 3.87 (s, 3H), 3.35 (s, 3H), 3.03 (d, J=4.4 Hz, 3H), 2.11-2.08 (m, 1H), 0.93-0.84 (m, 4H).

### Example 693: Synthesis of N-(5-((1-(3-methoxypropyl)-1H-benzo[d]imidazol-6-yl)ethynyl)-8-(methylamino)-2,7-naphthyridin-3-yl)cyclopropanecarboxamide

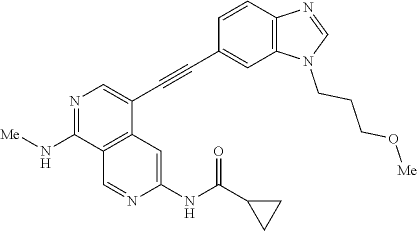 [SMILES: CNc1ncc(C#Cc2ccc3ncn(CCCOC)c3c2)c2cc(NC(=O)C3CC3)ncc12]

### Step 1: 6-bromo-1-(3-methoxypropyl)-1H-benzo[d]imidazole

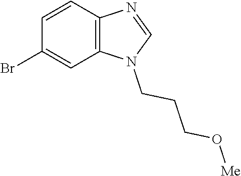 [SMILES: COCCCn1cnc2ccc(Br)cc21]

A solution of 5-bromo-N¹43-methoxypropyl)benzene-1,2-diamine (400.3 mg; 1.534 mmol; 1.00 eq.) in trimethoxymethane (4 mL) was stirred at 100° C. for 2 hours under nitrogen atmosphere. The desired product was detected via LCMS. The mixture was allowed to cool down to room temperature. The resulting mixture was concentrated under reduced pressure. The residue was purified by flash chromatography on silica gel column using 10-30% of EtOAc in petroleum ether as eluent to provide 6-bromo-1-(3-methoxypropyl)-1H-benzo[d]imidazole as a white solid (180.3 mg, 43.3%). LCMS (ESI) m/z 269.0, [M+H]⁺.

### Step 2: N-(5-((1-(3-methoxypropyl)-1H-benzo[d]imidazol-6-yl)ethynyl)-8-(methylamino)-2,7-naphthyridin-3-yl)cyclopropanecarboxamide

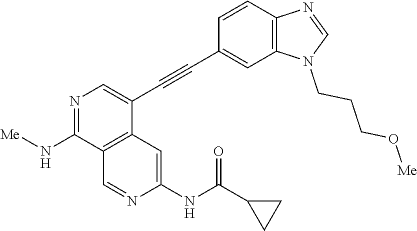 [SMILES: CNc1ncc(C#Cc2ccc3ncn(CCCOC)c3c2)c2cc(NC(=O)C3CC3)ncc12]

N-(5-((1-(3-methoxypropyl)-1H-benzo[d]imidazol-6-yl)ethynyl)-8-(methylamino)-2,7-naphthyridin-3-yl)cyclopropanecarboxamide was synthesized using a similar procedure that was previously described in Example 595 by using N-(5-ethynyl-8-(methylamino)-2,7-naphthyridin-3-yl)cyclopropanecarboxamide and 6-bromo-1-(3-methoxypropyl)-1H-benzo[d]imidazole as the starting material. LCMS (ESI) m/z 455.2, [M+H]⁺. ¹H NMR (300 MHz, DMSO-d₆) δ 11.08 (s, 1H), 9.38 (s, 1H), 8.77 (s, 1H), 8.35-8.26 (m, 3H), 7.89 (s, 1H), 7.70 (d, J=8.1 Hz, 1H), 7.43-7.35 (m, 1H), 4.35 (t, J=6.9 Hz, 2H), 3.30 (t, J=6.9 Hz, 2H), 3.24 (s, 3H), 3.03 (d, J=4.4 Hz, 3H), 2.10-2.04 (m, 3H), 0.97-0.80 (m, 4H).

### Example 694: Synthesis of N-(5-((1-(3-cyanopropyl)-1H-benzo[d]imidazol-6-yl)ethynyl)-8-(methylamino)-2,7-naphthyridin-3-yl)cyclopropanecarboxamide

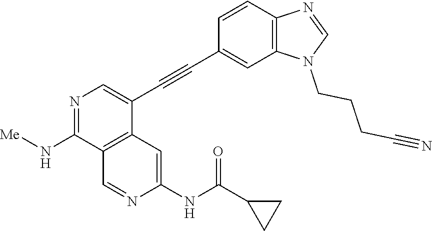 [SMILES: CNc1ncc(C#Cc2ccc3ncn(CCCC#N)c3c2)c2cc(NC(=O)C3CC3)ncc12]

### Step 1: 4-(6-bromo-1H-benzo[d]imidazol-1-yl)butanenitrile

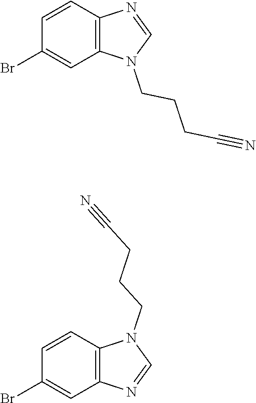 [SMILES: N#CCCCn1cnc2ccc(Br)cc21 ; N#CCCCn1cnc2cc(Br)ccc21]

To a stirred solution of 6-bromo-1H-benzo[d]imidazole (1.0 g; 5.076 mmol; 1.00 eq.) and Cs₂CO₃ (4.96 g; 15.228 mmol; 3.00 eq.) in DMF (16 mL) was added 4-bromobutanenitrile (1.50 g; 10.152 mmol; 2.00 eq.) under nitrogen atmosphere. The reaction was stirred at room temperature for 1 hour. The desired product was detected via LCMS. The resulting mixture was diluted with EtOAc (200 mL) and washed by brine (3×20 mL). The organic layer was dried over anhydrous sodium sulfate. After filtration, the filtrate was concentrated under reduced pressure. The residue was purified by flash chromatography on silica gel column using 20-70% of EtOAc in petroleum ether as eluent to provide the mixture. The mixture was separated by Achiral-SFC (Column: DAICEL DCpak P4VP 3×25 cm, 5 μm; Mobile Phase A: CO₂, Mobile Phase B: MeOH (0.1% 2 M NH₃-MeOH); Flow rate: 60 mL/min; Gradient: isocratic 12% B; Wave Length: 254 nm; R_{T1} (min): 8.37; R_{T2} (min): 10.27) to provide 4-(6-bromo-1H-benzo[d]imidazol-1-yl)butanenitrile as a white oil (345.0 mg, 25.7%) and 4-(5-bromo-1H-benzo[d]imidazol-1-yl)butanenitrile as a white oil (419.0 mg, 31.2%). LCMS (ESI) m/z 264.0.

### Step 2: N-(5-((1-(3-cyanopropyl)-1H-benzo[d]imidazol-6-yl)ethynyl)-8-(methylamino)-2,7-naphthyridin-3-yl)cyclopropanecarboxamide

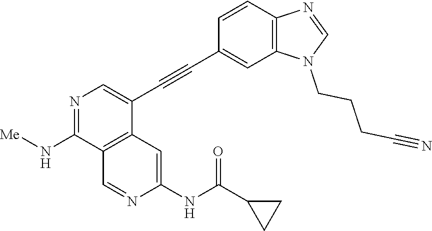 [SMILES: CNc1ncc(C#Cc2ccc3ncn(CCCC#N)c3c2)c2cc(NC(=O)C3CC3)ncc12]

N-(54(1-(3-cyanopropyl)-1H-benzo[d]imidazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 4-(6-bromo-1H-benzo[d]imidazol-1-yl)butanenitrile as the starting material. LCMS (ESI) m/z 450.1, [M+H]⁺. ¹H NMR (400 MHz, DMSO-d₆) δ 11.08 (s, 1H), 9.38 (s, 1H), 8.75 (s, 1H), 8.45-8.30 (m, 3H), 7.94 (s, 1H), 7.71 (d, J=8.4 Hz, 1H), 7.42 (d, J=8.4 Hz, 1H), 4.38 (t, J=7.2 Hz, 2H), 3.03 (d, J=4.4 Hz, 3H), 2.59-2.53 (m, 2H), 2.24-2.13 (m, 2H), 2.13-2.05 (m, 1H), 0.95-0.81 (m, 4H).

### Example 695: Synthesis of N-(5-((1-(3-cyanopropyl)-1/1-benzo[d]imidazol-5-yl)ethynyl)-8-(methylamino)-2,7-naphthyridin-3-yl)cyclopropanecarboxamide

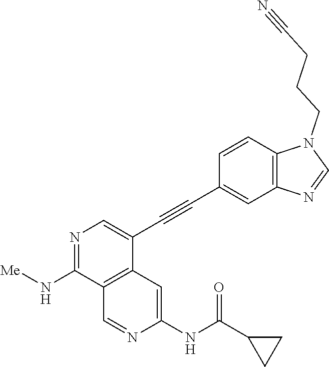 [SMILES: CNc1ncc(C#Cc2ccc3c(c2)ncn3CCCC#N)c2cc(NC(=O)C3CC3)ncc12]

N-(5-((1-(3-cyanopropyl)-1H-benzo[d]imidazol-5-yl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 4-(5-bromo-1H-benzo[d]imidazol-1-yl)butanenitrile as the starting material. LCMS (ESI) m/z 450.1, [M+H]⁺. ¹H NMR (400 MHz, DMSO-d₆) δ 11.08 (s, 1H), 9.38 (s, 1H), 8.72 (s, 1H), 8.35 (s, 1H), 8.33-8.29 (m, 1H), 8.29 (s, 1H), 7.85 (s, 1H), 7.73 (d, J=8.3 Hz, 1H), 7.50 (d, J=8.3 Hz, 1H), 4.38 (t, J=7.2 Hz, 2H), 3.03 (d, J=4.4 Hz, 3H), 2.59-2.53 (m, 2H), 2.20-2.06 (m, 3H), 0.93-0.80 (m, 4H).

### Example 696: Synthesis of N-(5-((2-(hydroxymethyl)-2,3-dihydrobenzo[b][1,4]dioxin-6-yl)ethynyl)-8-(methylamino)-2,7-naphthyridin-3-yl)cyclopropanecarboxamide

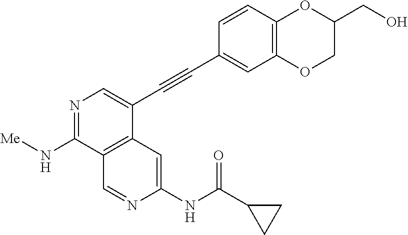 [SMILES: CNc1ncc(C#Cc2ccc3c(c2)OCC(CO)O3)c2cc(NC(=O)C3CC3)ncc12]

### Step 1: 4-bromo-2-(oxiran-2-ylmethoxy)benzaldehyde

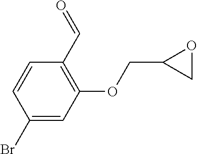 [SMILES: O=Cc1ccc(Br)cc1OCC1CO1]

A solution of 4-bromo-2-hydroxybenzaldehyde (2.00 g; 9.940 mmol; 1.00 eq.), 2-(chloromethyl)oxirane (1.10 g; 11.928 mmol; 1.20 eq.) and K₂CO₃ (2.75 g; 19.880 mmol; 2.00 eq.) in DMF (20 mL) was stirred at 80° C. for 16 hours. The desired product was detected via LCMS. The resulting mixture was diluted with EtOAc (3×50 mL) and washed with water (2×30 mL). The organic layer was dried over anhydrous Na₂SO₄. After filtration, the filtrate was concentrated under reduced pressure. The residue was purified by flash chromatography on silica gel column using 20-35% of EtOAc in petroleum ether as eluent to provide the crude product. The crude product was purified by flash chromatography on pre-packed C18 column using 70-90% of MeOH in water (0.1% trifluoroacetic acid) as eluent to provide 4-bromo-2-(oxiran-2-ylmethoxy)benzaldehyde as a yellow solid (726.0 mg, 23.5%). LCMS (ESI) m/z 257.0, [M+H]⁺.

### Step 2: 4-bromo-2-(oxiran-2-ylmethoxy)phenyl formate

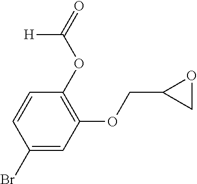 [SMILES: O=COc1ccc(Br)cc1OCC1CO1]

A solution of 4-bromo-2-(oxiran-2-ylmethoxy)benzaldehyde (626.1 mg; 2.430 mmol; 1.00 eq.) and m-CPBA (840.1 mg; 4.860 mmol; 2.00 eq.) in CH₂Cl₂ (15 mL) was stirred at 50° C. for 5 hours. The desired product was detected via LCMS. The reaction was quenched by the addition of aqueous Na₂S₂O₈ solution (12 mL). The resulting mixture was extracted with CH₂Cl₂ (3×10 mL). The combined organic layers were washed with saturated sodium bicarbonate (3×6 mL), dried over anhydrous Na₂SO₄. After filtration, the filtrate was concentrated under reduced pressure to afford 4-bromo-2-(oxiran-2-ylmethoxy)phenyl formate as a yellow oil (547.2 mg, 48.3%). LCMS (ESI) m/z 273.1, [M+H]⁺.

### Step 3: (6-bromo-2,3-dihydrobenzo[b][1,4]dioxin-2-yl)methanol

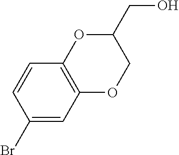 [SMILES: OCC1COc2cc(Br)ccc2O1]

A solution of 4-bromo-2-(oxiran-2-ylmethoxy)phenyl formate (547.2 mg; 2.000 mmol; 1.00 eq.) and NaOH (120.1 mg; 3.000 mmol; 1.50 eq.) in a mixture solvent of THE/water. (4:1, 12.5 mL) was stirred at room temperature for 2 hours. The desired product was detected via LCMS. The mixture was acidified to pH=5 with HCl (2 M). The resulting mixture was extracted with EtOAc (3×10 mL). The combined organic layers were washed with brine (2×10 mL), dried over anhydrous Na₂SO₄. After filtration, the filtrate was concentrated under reduced pressure to afford (6-bromo-2,3-dihydrobenzo[b][1,4]dioxin-2-yl)methanol as a yellow oil (377.2 mg, 68.8%). LCMS (ESI) m/z 245.1, [M+H]⁺.

### Step 4: N-(5-((2-(hydroxymethyl)-2,3-dihydrobenzo[b][1,4]dioxin-6-yl)ethynyl)-8-(methylamino)-2,7-naphthyridin-3-yl)cyclopropanecarboxamide

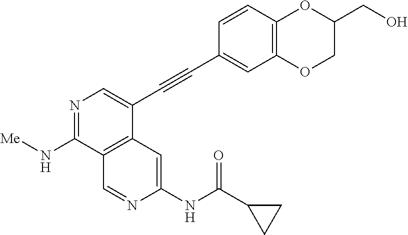 [SMILES: CNc1ncc(C#Cc2ccc3c(c2)OCC(CO)O3)c2cc(NC(=O)C3CC3)ncc12]

N-(5-((2-(hydroxymethyl)-2,3-dihydrobenzo[b][1,4]dioxin-6-yl)ethynyl)-8-(methylamino)-2,7-naphthyridin-3-yl)cyclopropanecarboxamide was synthesized using a similar procedure that was previously described in Example 595, by using-(5-ethynyl-8-(methylamino)-2,7-naphthyridin-3-yl)cyclopropanecarboxamide and (6-bromo-2,3-dihydrobenzo[b][1,4]dioxin-2-yl)methanol as the starting material. LCMS (ESI) m/z 431.2, [M+H]⁺. ¹H NMR (400 MHz, DMSO-d₆) δ 11.07 (s, 1H), 9.36 (s, 1H), 8.62 (s, 1H), 8.31-8.25 (m, 1H), 8.24 (s, 1H), 7.13-7.00 (m, 2H), 6.96-6.92 (m, 1H), 5.10 (d, J=5.6 Hz, 1H), 4.41-4.32 (m, 1H), 4.29-4.18 (m, 1H), 4.11-4.00 (m, 1H), 3.71-3.55 (m, 2H), 3.01 (d, J=4.4 Hz, 3H), 2.15-2.03 (m, 1H), 0.96-0.79 (m, 4H).

### Examples 697, 698 and 699

### Synthesis of N-(5-((2-(methoxymethyl)-2,3-dihydrobenzo[b][1,4]dioxin-6-yl)ethynyl)-8-(methylamino)-2,7-naphthyridin-3-yl)cyclopropanecarboxamide (Example 697), (S)—N-(5-((2-(methoxymethyl)-2,3-dihydrobenzo[b][1,4]dioxin-6-yl)ethynyl)-8-(methylamino)-2,7-naphthyridin-3-yl)cyclopropanecarboxamide (Example 698) and (R)—N-(5-((2-(methoxymethyl)-2,3-dihydrobenzo[b][1,4]dioxin-6-yl)ethynyl)-8-(methyl amino)-2,7-naphthyridin-3-yl)cyclopropanecarboxamide (Example 699)

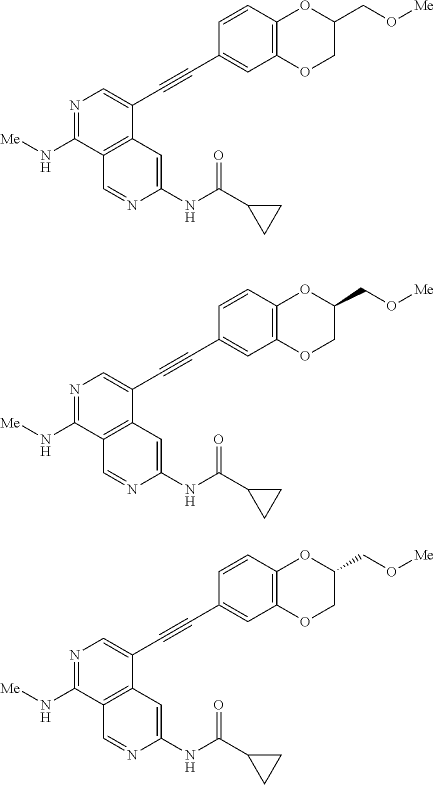

### Step 1: 6-bromo-2-(methoxymethyl)-2,3-dihydrobenzo[b][1,4]dioxine

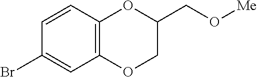 [SMILES: COCC1COc2cc(Br)ccc2O1]

To a stirred solution of (6-bromo-2,3-dihydro-1,4-benzodioxin-2-yl)methanol (Example 696, Step 3) (327.3 mg; 1.336 mmol; 1.00 eq.) in THF (6 mL) was added NaH (60% dispersion in mineral oil, 80.1 mg; 2.004 mmol; 1.5 eq.) at 0° C. under nitrogen atmosphere. The reaction mixture was stirred at room temperature for 0.5 hour. To the above solution was MeI (284.3 mg; 2.004 mmol; 1.50 eq.), the resulting solution was stirred at room temperature for 4 hours. The desired product was detected via LCMS. The reaction was quenched by the addition of water (10 mL). The resulting mixture was diluted with EtOAc (50 mL) and washed with brine (3×5 mL). The organic layer was concentrated under vacuum. The residue was purified by flash chromatography on silica gel column using 5-10% of EtOAc in petroleum ether as eluent to provide 6-bromo-2-(methoxymethyl)-2,3-dihydrobenzo[b][1,4]dioxine as a yellow oil (374.5 mg, 43.6%). LCMS (ESI) m/z 259.0, [M+H]⁺.

### Step 2: N-(5-((2-(methoxymethyl)-2,3-dihydrobenzo[b][1,4]dioxin-6-yl)ethynyl)-8-(methylamino)-2,7-naphthyridin-3-yl)cyclopropanecarboxamide

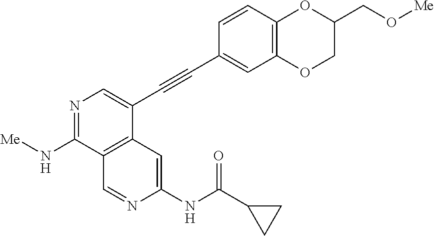 [SMILES: CNc1ncc(C#Cc2ccc3c(c2)OCC(COC)O3)c2cc(NC(=O)C3CC3)ncc12]

N-(5-((2-(methoxymethyl)-2,3-dihydrobenzo[b][1,4]dioxin-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-2-(methoxymethyl)-2,3-dihydrobenzo[b][1,4]dioxine as the starting material. LCMS (ESI) m/z 445.2, [M+H]⁺. ¹H NMR (400 MHz, DMSO-d₆) δ 11.06 (s, 1H), 9.36 (s, 1H), 8.61 (s, 1H), 8.42-8.31 (m, 1H), 8.23 (s, 1H), 7.05-7.03 (m, 2H), 6.95 (d, J=8.8 Hz, 1H), 4.38-4.34 (m, 2H), 4.07-4.04 (m, 1H), 3.61 (d, J=4.4 Hz, 2H), 3.33 (s, 3H), 3.01 (d, J=4.4 Hz, 3H), 2.10-2.04 (m, 1H), 0.90-0.84 (m, 4H).

### Step 3: (S)—N-(5-((2-(methoxymethyl)-2,3-dihydrobenzo[b][1,4]dioxin-6-yl)ethynyl)-8-(methylamino)-2,7-naphthyridin-3-yl)cyclopropanecarboxamide and (R)N-(5-((2-(methoxymethyl)-2,3-dihydrobenzo[b][1,4]dioxin-6-yl)ethynyl)-8-(methylamino)-2,7-naphthyridin-3-yl)cyclopropanecarboxamide

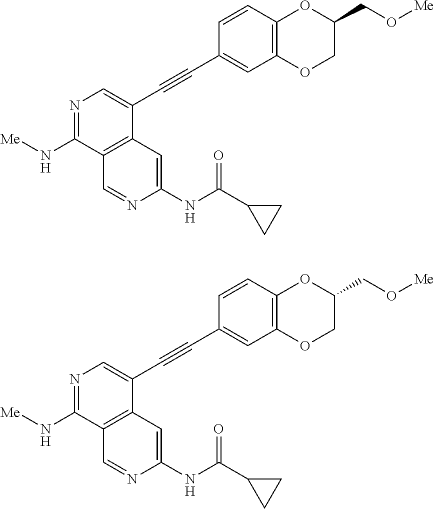 [SMILES: CNc1ncc(C#Cc2ccc3c(c2)OC[C@H](COC)O3)c2cc(NC(=O)C3CC3)ncc12 ; CNc1ncc(C#Cc2ccc3c(c2)OC[C@@H](COC)O3)c2cc(NC(=O)C3CC3)ncc12]

N-(5-((2-(methoxymethyl)-2,3-dihydrobenzo[b][1,4]dioxin-6-yl)ethynyl)-8-(methylamino)-2,7-naphthyridin-3-yl)cyclopropanecarboxamide was separated by Chiral-HPLC (column, CHIRALPAK IH, 3×25 cm, 5 μm; Mobile Phase A: CO₂, Mobile Phase B: IPA:CH₂Cl₂=1:1 (0.1% 2 M NH₃-MeOH); Flow rate: 140 mL/min; Gradient: isocratic 40% B; Wave Length: 254 nm; R_{T1}(min): 14.18; R_{T2}(min): 15.98) to provide (S)—N-(5-((2-(methoxymethyl)-2,3-dihydrobenzo[b][1,4]dioxin-6-yl)ethynyl)-8-(methylamino)-2,7-naphthyridin-3-yl)cyclopropanecarboxamide (Example 698, the faster peak) as yellow solid and (R)—N-(5-((2-(methoxymethyl)-2,3-dihydrobenzo[b][1,4]dioxin-6-yl)ethynyl)-8-(methylamino)-2,7-naphthyridin-3-yl)cyclopropanecarboxamide (Example 699, the slower peak) as a yellow solid. The two configurations are arbitrarily assigned. LCMS (ESI) m/z 445.2, [M+H]⁺.

HNMR for Example 698: ¹H NMR (400 MHz, DMSO-d₆) δ 11.06 (s, 1H), 9.36 (s, 1H), 8.61 (s, 1H), 8.42-8.31 (m, 1H), 8.23 (s, 1H), 7.05-7.03 (m, 2H), 6.95 (d, J=8.8 Hz, 1H), 4.38-4.34 (m, 2H), 4.07-4.04 (m, 1H), 3.61 (d, J=4.4 Hz, 2H), 3.33 (s, 3H), 3.01 (d, J=4.4 Hz, 3H), 2.10-2.04 (m, 1H), 0.90-0.84 (m, 4H).

HNMR for Example 699: ¹H NMR (400 MHz, DMSO-d₆) δ 11.06 (s, 1H), 9.36 (s, 1H), 8.61 (s, 1H), 8.42-8.31 (m, 1H), 8.23 (s, 1H), 7.05-7.03 (m, 2H), 6.95 (d, J=8.8 Hz, 1H), 4.38-4.34 (m, 2H), 4.07-4.04 (m, 1H), 3.61 (d, J=4.4 Hz, 2H), 3.33 (s, 3H), 3.01 (d, J=4.4 Hz, 3H), 2.10-2.04 (m, 1H), 0.90-0.84 (m, 4H).

### Example 700: Synthesis of N-(5-((1,5-dimethyl-1H-benzo[d][1,2,3]triazol-6-yl)ethynyl)-8-(methylamino)-2,7-naphthyridin-3-yl)cyclopropanecarboxamide

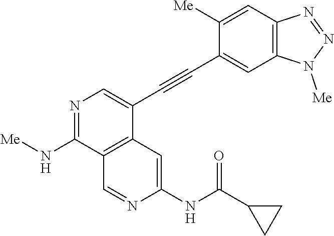 [SMILES: CNc1ncc(C#Cc2cc3c(cc2C)nnn3C)c2cc(NC(=O)C3CC3)ncc12]

### Step 1: 5-bromo-N,4-dimethyl-2-nitroaniline

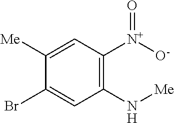 [SMILES: CNc1cc(Br)c(C)cc1[N+](=O)[O-]]

To a stirred solution of 1-bromo-5-fluoro-2-methyl-4-nitrobenzene (2.00 g; 8.404 mmol; 1.00 eq.) and methylamine hydrochloride (0.60 g; 9.244 mmol; 1.10 eq.) in DMF (10 mL) was added K₂CO₃ (3.50 g; 25.212 mmol; 3.00 eq.) at room temperature. The resulting mixture was stirred at room temperature for 12 hours under nitrogen atmosphere. The desired product was detected via LCMS. The resulting mixture was diluted with water (20 mL) and extracted with EtOAc (3×50 mL). The combined organic layers were washed with water (3×20 mL), dried over anhydrous Na2SO4. After filtration, the filtrate was concentrated under reduced pressure. The residue was purified by flash chromatography on silica gel column using 10-30% of EtOAc in petroleum ether as eluent to provide 5-bromo-N,4-dimethyl-2-nitroaniline as a yellow solid (1.80 g, 86.0%). LCMS (ESI) m/z 245.0, [M+H]⁺.

### Step 2: 5-bromo N¹,4-dimethylbenzene-1,2-diamine

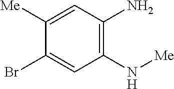 [SMILES: CNc1cc(Br)c(C)cc1N]

A solution of 5-bromo-N,4-dimethyl-2-nitroaniline (500.0 mg; 2.040 mmol; 1.00 eq.) and Fe (1139.3 mg; 20.400 mmol; 10.00 eq.) in AcOH (5 mL) was stirred at 70° C. for 3 hours under nitrogen atmosphere. The desired product was detected via LCMS. The mixture was allowed to cool down to room temperature. The resulting mixture was diluted with water (20 mL) and neutralized to pH 7 with saturated NaHCO₃ solution. The resulting mixture was extracted with EtOAc (3×30 mL). The combined organic layers were washed with water (3×30 mL), dried over anhydrous Na₂SO₄. After filtration, the filtrate was concentrated under reduced pressure to afford 5-bromo N¹,4-dimethylbenzene-1,2-diamine as an off-white solid (420.0 mg, 95.0%). LCMS (ESI) m/z 215.0, [M+H]⁺.

### Step 3: 6-bromo-1,5-dimethyl-1H-benzo[d][1,2,3]triazole

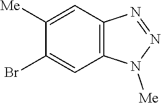 [SMILES: Cc1cc2nnn(C)c2cc1Br]

To a stirred solution of 5-bromo-N¹,4-dimethylbenzene-1,2-diamine (500.0 mg; 2.325 mmol; 1.00 eq.) in HCl (12 M, 10 mL) was added a solution of NaNO₂ (240.6 mg; 3.488 mmol; 1.50 eq.) in water (2 mL) dropwise at 0° C. The resulting solution was stirred at room temperature for 1 hour. The desired product was detected via LCMS. The resulting mixture was diluted with water (20 mL) and neutralized to pH=7 with saturated NaHCO₃ solution. The resulting mixture was extracted with EtOAc (3×30 mL). The combined organic layers were washed with water (3×30 mL), dried over anhydrous Na₇SO₄. After filtration, the filtrate was concentrated under reduced pressure to afford 6-bromo-1,5-dimethyl-1H-benzo[d][1,2,3]triazole as a light yellow solid (250.0 mg, 47.0%). LCMS (ESI) m/z 226.0, [M+H]⁺.

### Step 4: N-(5-((1,5-dimethyl-1H-benzo[d][1,2,3]triazol-6-yl)ethynyl)-8-(methylamino)-2,7-naphthyridin-3-yl)cyclopropanecarboxamide

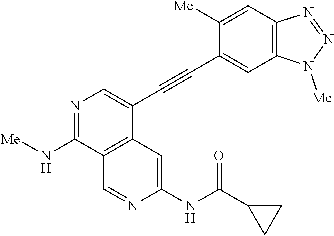 [SMILES: CNc1ncc(C#Cc2cc3c(cc2C)nnn3C)c2cc(NC(=O)C3CC3)ncc12]

N-(5-((1,5-dimethyl-1H-benzo[d][1,2,3]triazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-1,5-dimethyl-1H benzo[d][1,2,3]triazole as the starting material. LCMS (ESI) m/z 412.2, [M+H]⁺. ¹H NMR (400 MHz, DMSO-d₆) δ 11.11 (s, 1H), 9.40 (s, 1H), 8.76 (s, 1H), 8.46-8.36 (m, 1H), 8.33 (s, 1H), 8.1.2 (s, 1H), 7.96 (s, 1H), 4.32 (s, 3H), 3.04 (d, J=4.4 Hz, 3H), 2.68 (s, 3H), 2.14-2.05 (m, 1H), 0.94-0.81 (m, 4H).

### Example 701: Synthesis of N-(8-(methylamino)-5-((4-((3-methyloxetan-3-yl)methoxy)phenyl)ethynyl)-2,7-naphthyridin-3-yl)cyclopropanecarboxamide

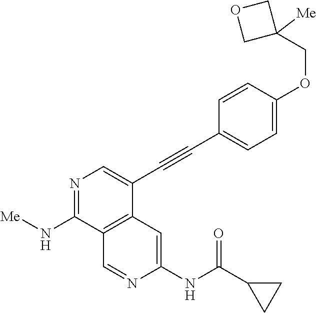 [SMILES: CNc1ncc(C#Cc2ccc(OCC3(C)COC3)cc2)c2cc(NC(=O)C3CC3)ncc12]

### Step 1: 3-((4-bromophenoxy)methyl)-3-methyloxetane

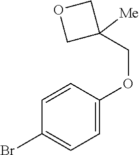 [SMILES: CC1(COc2ccc(Br)cc2)COC1]

To a stirred solution of 4-bromophenol (300.0 mg; 1.734 mmol; 1.00 eq.), Cs₂CO₃ (1.69 g; 5.202 mmol; 3.00 eq.) in DMF (5 mL) was added 3-(bromomethyl)-3-methyloxetane (572.3 mg; 3.468 mmol; 2.00 eq.) dropwise at room temperature under nitrogen atmosphere. The resulting mixture was stirred at 50° C. for 2 hours. The desired product was detected via LCMS. The resulting mixture was diluted with brine (20 mL) and extracted with EtOAc (2×50 mL). The combined organic layers were washed with brine (3×10 mL), dried over anhydrous Na₂SO₄. After filtration, the filtrate was concentrated under reduced pressure to provide 3-((4-bromophenoxy)methyl)-3-methyloxetane as a yellow solid (300 mg, crude). The crude product was used in the next step directly without further purification. LCMS (ESI) m/z 257.0, [M+H]⁺.

### Step 2:11′-(8-(methylamino)-5-((4-((3-methyloxetan-3-yl)methoxy)phenyl)ethynyl)-2,7-naphthyridin-3-yl)cyclopropanecarboxamide

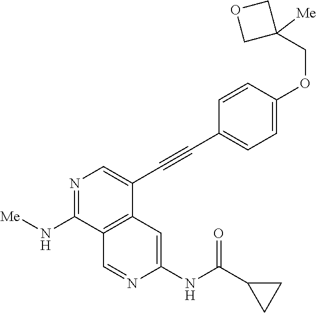 [SMILES: CNc1ncc(C#Cc2ccc(OCC3(C)COC3)cc2)c2cc(NC(=O)C3CC3)ncc12]

N-(8-(methylamino)-5-((4-((3-methyloxetan-3-yl)methoxy)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-((4-bromophenoxy)methyl)-3-methyloxetane as the starting material. LCMS (ESI) m/z 443.2, [M+H]⁺. ¹H NMR (400 MHz, DMSO-d₆) δ 11.07 (s, 1H), 9.37 (s, 1H), 8.65 (s, 1H), 8.37-8.18 (m, 2H), 7.51 (d, J=8.4 Hz, 2H), 7.06 (d, J=8.4 Hz, 2H), 4.51 (d, J=6.0 Hz, 2H), 4.32 (d, J=6.0 Hz, 2H), 4.11 (s, 2H), 3.02 (d, J=4.4 Hz, 3H), 2.12-2.02 (m, 1H), 1.38 (s, 3H), 0.91-0.81 (m, 4H).

### Example 702: Synthesis of N-(8-(methylamino)-5-((4-(oxetan-2-ylmethoxy)phenyl)ethynyl)-2,7-naphthyridin-3-yl)cyclopropanecarboxamide

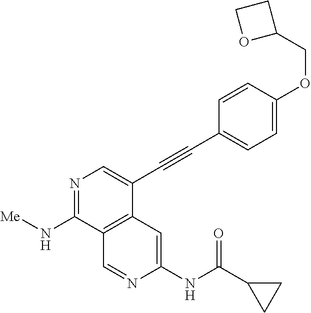 [SMILES: CNc1ncc(C#Cc2ccc(OCC3CCO3)cc2)c2cc(NC(=O)C3CC3)ncc12]

### Step 1: oxetan-2-ylmethyl methanesulfonate

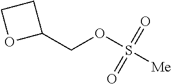

To a solution of oxetan-2-ylmethanol (200.0 mg; 2.271 mmol; 1.00 eq.) and DIPEA (293.3 mg, 2.270 mmol, 1.00 eq.) in CH₂Cl₂ (4 mL) was added methanesulfonic anhydride (790.8 mg, 4.540 mmol, 2.00 eq.) drop/vise at 0° C. under nitrogen atmosphere. The resulting mixture was stirred at room temperature for 2 hours under nitrogen atmosphere. The reaction was quenched by the addition of water (5 mL). The resulting mixture was extracted with CH₂Cl₂ (3×10 mL). The combined organic layers were washed with brine (2×5 mL), dried over anhydrous Na₂SO₄. After filtration, the filtrate was concentrated under reduced pressure to afford oxetan-2-ylmethyl methanesulfonate as a brown oil (550.1 mg, crude). The crude product was used in the next step directly without further purification. LCMS (ESI) m/z 167.0, [M+H]⁺.

### Step 2: 2-((4-bromophenoxy)methyl)oxetane

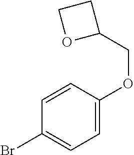 [SMILES: Brc1ccc(OCC2CCO2)cc1]

A solution of 4-bromophenol (286.2 mg; 1.651 mmol; 1.00 eq.), oxetan-2-ylmethyl methanesulfonate (550.1 mg; 3.302 mmol; 2.00 eq.) and Cs₂CO₃ (1.61 g; 4.963 mmol; 3.00 eq.) in DMF (3 mL) was stirred at 50° C. for 2 hours under nitrogen atmosphere. The resulting mixture was diluted with EtOAc (50 mL) and washed with brine (2×10 mL). The organic layer was dried over anhydrous Na₂SO₄. After filtration, the filtrate was concentrated under reduced pressure. The residue was purified by flash chromatography on silica gel column using 0-15% of EtOAc in petroleum ether as eluent to provide 2-((4-bromophenoxy)methyl)oxetane as an off-white oil (150.0 mg, 37.1%). LCMS (ESI) m/z 243.0, [M+H]⁺.

### Step 3: N-(8-(methylamino)-5-((4-(oxetan-2-ylmethoxy)phenyl)ethynyl)-2,7-naphthyridin-3-yl)cyclopropanecarboxamide

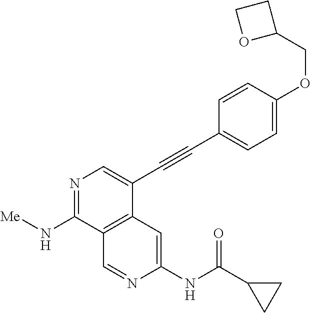 [SMILES: CNc1ncc(C#Cc2ccc(OCC3CCO3)cc2)c2cc(NC(=O)C3CC3)ncc12]

N-(8-(methyl amino)-5-((4-(oxetan-2-ylmethoxy)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 2-((4-bromophenoxy)methyl)oxetane as the starting material. LCMS (ESI) m/z 429.2, [M+H]⁺. ¹H NMR (400 MHz, DMSO-d₆) δ 11.10 (s, 1H), 9.37 (s, 1H), 8.65 (s, 1H), 8.32-8.26 (m, 1H), 8.25 (s, 1H), 7.55-7.43 (m, 2H), 7.11-6.96 (m, 2H), 4.15 (t, J=6.4 Hz, 2H), 3.15-3.05 (m, 1H), 3.02 (d, J=4.4 Hz, 3H), 2.82-2.73 (m, 1H), 2.63-2.55 (m, 1H), 2.14-1.86 (m, 3H), 0.97-0.82 (m, 4H).

### Example 703: Synthesis of (R)—N-(5-((4-(2-methoxypropoxy)phenyl)ethynyl)-8-(methylamino)-2,7-naphthyridin-3-yl)cyclopropanecarboxamide

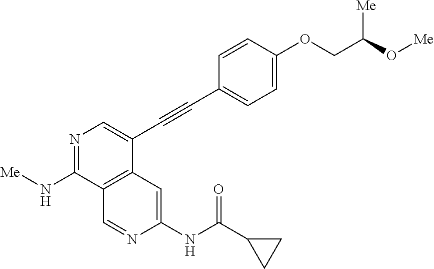 [SMILES: CNc1ncc(C#Cc2ccc(OC[C@@H](C)OC)cc2)c2cc(NC(=O)C3CC3)ncc12]

### Step 1: (R)-2-methoxypropyl methanesulfonate

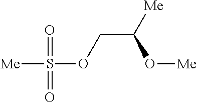

To a stirred solution of (R)-2-methoxypropan-1-ol. (100.0 mg; 1.110 mmol; 1.00 eq.) and DIEA. (143.0 mg; 1.106 mmol; 1.00 eq.) in CH₂Cl₂ (4 mL) was added methanesulfonic anhydride (386.0 mg; 2.216 mmol; 2.00 eq.) under nitrogen atmosphere. The resulting solution was stirred at room temperature for 2 hours. The reaction was concentrated under vacuum to afford (R)-2-methoxypropyl methanesulfonate as a brown oil (186.0 mg crude). This crude product was used in the next step directly without further purification.

### Step 2: (R)-1-bromo-4-(2-methoxypropoxy)benzene

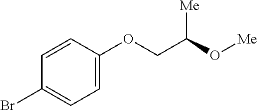 [SMILES: CO[C@H](C)COc1ccc(Br)cc1]

To a stirred solution of 4-bromophenol (95.0 mg; 0.549 mmol; 1.00 eq.) and Cs₂CO; (539.0 mg; 1.654 mmol; 3.01 eq.) in DMF (3 mL) was added (R)-2-methoxypropyl methanesulfonate (186.0 mg; 1.106 mmol; 2.01 eq.) under nitrogen atmosphere. The resulting solution was stirred at 50° C. for 2 hours. The resulting mixture was diluted with water (30 mL), extracted with EtOAc (3×30 mL). The combined organic layers were washed with brine (3×10 mL), dried over anhydrous Na₂SO₄. After filtration, the filtrate was concentrated under reduced pressure. The residue was purified by flash chromatography on silica gel column using 5-10% of MeOH in CH₂Cl₂ as eluent to provide (R)-1-bromo-4-(2-methoxypropoxy)benzene (80.0 mg, 59.4%) as a yellow oil.

### Step 3: (R)—N-(5-((4-(2-methoxypropoxy)phenyl)ethynyl)-8-(methylamino)-2,7-naphthyridin-3-yl)cyclopropanecarboxamide

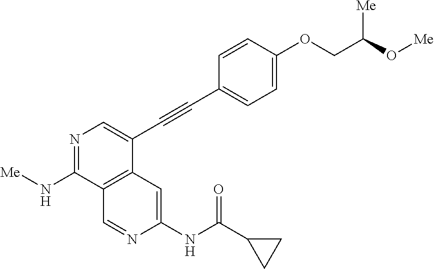 [SMILES: CNc1ncc(C#Cc2ccc(OC[C@@H](C)OC)cc2)c2cc(NC(=O)C3CC3)ncc12]

(R)—N-(5-((4-(2-methoxypropoxy)phenyl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and (R)-1-bromo-4-(2-methoxypropoxy)benzene as the starting material. LCMS (ESI) m/z 431.2, [M+H]⁺. ¹H NMR (400 MHz, DMSO-d₆) δ 11.07 (s, 1H), 9.37 (s, 1H), 8.64 (s, 1H), 8.31-8.27 (m, 1H), 8.25 (s, 1H), 7.48 (d, J=8.4 Hz, 2H), 7.02 (d, J=8.4 Hz, 2H), 3.98 (d, J=5.2 Hz, 2H), 3.72-3.68 (m, 1H), 3.32 (s, 3H), 3.02 (d, J=4.4 Hz, 3H), 2.12-2.08 (m, 1H), 1.19 (d, J=6.0 Hz, 3H), 0.92-0.81 (m, 4H).

### Example 704: Synthesis of (S)—N-(5-((4-(2-methoxypropoxy)phenyl)ethynyl)-8-(methylamino)-2,7-naphthyridin-3-yl)cyclopropanecarboxamide

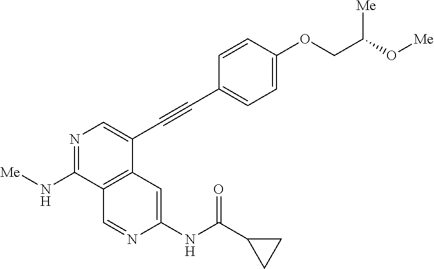 [SMILES: CNc1ncc(C#Cc2ccc(OC[C@H](C)OC)cc2)c2cc(NC(=O)C3CC3)ncc12]

### Step 1: (S)-2-methoxypropyl methanesulfonate

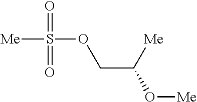

To a stirred solution of (S)-2-methoxypropan-1-ol (100.0 mg; 1.110 mmol; 1.00 eq.) and DIEA. (143.0 mg; 1.106 mmol; 1.00 eq.) in CH₂Cl₂ (4 mL) was added methanesulfonic anhydride (386.0 mg; 2.216 mmol; 2.00 eq.) at 0° C. The reaction was stirred at room temperature for 2 hours under nitrogen atmosphere. The reaction was quenched with water (1 mL). The resulting mixture was diluted with CH₂Cl₂ (50 mL) and washed with saturated NaHCO₃ solution (3×5 mL). The organic layer was dried over anhydrous Na₂SO₄. After filtration, the filtrate was concentrated under reduced pressure to afford (S)-2-methoxypropyl methanesulfonate as a white oil (282.0 mg, crude).

### Step 2: (S)-1-bromo-4-(2-methoxypropoxy)benzene

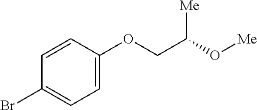 [SMILES: CO[C@@H](C)COc1ccc(Br)cc1]

A solution of 4-bromophenol (134.7 mg; 0.779 mmol; 1.00 eq.), Cs₂CO₃ (761.2 mg; 2.337 mmol; 3.00 eq.) and (S)-2-methoxypropyl methanesulfonate (262.0 mg; 1.558 mmol; 2.00 eq.) in DMF (4 mL) was stirred at 50° C. for 2 hours under nitrogen atmosphere. The desired product was detected via LCMS. The solvent was concentrated under vacuum. The residue was purified by flash chromatography on silica gel column using 10-50% of EtOAc in petroleum ether as eluent to provide (S)-1-bromo-4-(2-methoxypropoxy)benzene as a white oil (69.0 mg, 36.1%). LCMS (ESI) m. /z 245.1, [M+H]⁺.

### Step 3: (S)—N-(5-((4-(2-methoxypropoxy)phenyl)ethynyl)-8-(methylamino)-2,7-naphthyridin-3-yl)cyclopropanecarboxamide

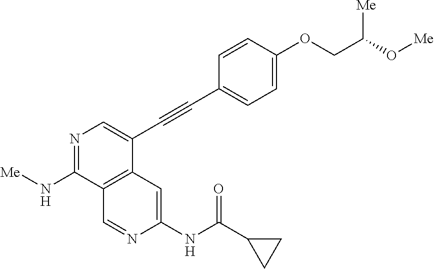 [SMILES: CNc1ncc(C#Cc2ccc(OC[C@H](C)OC)cc2)c2cc(NC(=O)C3CC3)ncc12]

(S)—N-(5-((4-(2-methoxypropoxy)phenyl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and (5)-1-bromo-4-(2-methoxypropoxy)benzene as the starting material. LCMS (ESI) m/z 431.2, [M+H]⁺. ¹H NMR (400 MHz, DMSO-d₆) δ 11.05 (s, 1H), 9.37 (s, 1H), 8.64 (s, 1H), 8.29-8.26 (m, 1H), 8.25 (s, 1H), 7.49 (d, J=8.8 Hz, 2H), 7.02 (d, J=8.8 Hz, 2H), 3.99 (d, J=4.8 Hz, 2H), 3.74-3.63 (m, 1H), 3.33 (s, 3H), 3.02 (d, J=4.4 Hz, 3H), 2.14-2.03 (m, 1H), 1.19 (d, J=6.4 Hz, 3H), 0.90-0.80 (m, 41-1).

### Example 705: Synthesis of N-(5-((4-((1-acetylazetidin-3-yl)oxy)phenyl)ethynyl)-8-(methylamino)-2,7-naphthyridin-3-yl)cyclopropanecarboxamide

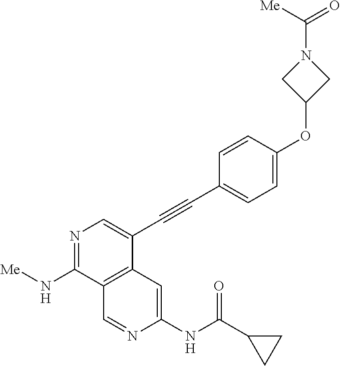 [SMILES: CNc1ncc(C#Cc2ccc(OC3CN(C(C)=O)C3)cc2)c2cc(NC(=O)C3CC3)ncc12]

### Step 1: tert-butyl 3-(4-bromophenoxy)azetidine-1-carboxylate

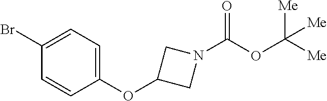 [SMILES: CC(C)(C)OC(=O)N1CC(Oc2ccc(Br)cc2)C1]

A solution of 4-bromophenol (1.00 g; 5.780 mmol; 1.00 eq.), tert-butyl 3-bromoazetidine-1-carboxylate (2.05 g; 8.682 mmol; 1.50 eq.) and Cs₂CO₃ (3.78 g; 11.602 mmol; 2.00 eq.) in DMF (15 mL) was stirred at 100° C. for 1 hour under nitrogen atmosphere. The resulting mixture was diluted with EtOAc (100 mL) and washed with brine (3×20 mL). The organic layer was dried by anhydrous Na₂SO₄. After filtration, the filtrate was concentrated under reduced pressure. The residue was purified by flash chromatography on silica gel column using 0-20% of EtOAc in petroleum ether as eluent to provide tort-butyl 3-(4-bromophenoxy)azetidine-1-carboxylate as a off-white solid (1.95 g, 92.5%).

### Step 2: 3-(4-bromophenoxy)azetidine hydrochloride

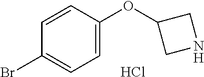 [SMILES: Brc1ccc(OC2CNC2)cc1 ; Cl]

To a stirred solution of tert-butyl 3-(4-bromophenoxy)azetidine-1-carboxylate (1.00 g; 3.047 mmol; 1.00 eq.) in MeOH (5 mL) was added HCl (gas) (4 M in 1,4-dioxane, 10 mL). The resulting solution was stirred at room temperature for 1 hour. The desired product was detected via LCMS. The solvent was concentrated under vacuum to afford 3-(4-bromophenoxy)azetidine hydrochloride as a off-white solid (1.10 g, crude). LCMS (ESI) m/z 228.0, [M+H]⁺.

### Step 3: 1-(3-(4-bromophenoxy)azetidin-1-yl)ethan-1-one

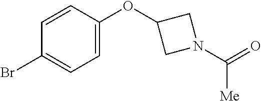 [SMILES: CC(=O)N1CC(Oc2ccc(Br)cc2)C1]

To a stirred solution of 3-(4-bromophenoxy)azetidine hydrochloride (1.10 g; 4.158 mmol; 1.00 eq.) and DIEA (1.34 g; 10.395 mmol; 2.50 eq.) in CH₂Cl₂ (15 mL) was added acetic anhydride (0.85 g; 8.316 mmol; 2.00 eq.) dropwise at 0° C. under nitrogen atmosphere. The resulting mixture was stirred at room temperature for 2 hours. The solvent was concentrated under vacuum. The residue was purified by flash chromatography on silica gel column using 0-30% of EtOAc in petroleum ether as eluent to provide 1-(3-(4-bromophenoxy)azetidin-1-yl)ethan-1-one as a white solid (1.10 g, 97.9%). LCMS (ESI) m/z 270.0, [M+H]⁺.

### Step 4: N-(5-((4-((1-acetylazetidin-3-yl)oxy)phenyl)ethynyl)-8-(methylamino)-2,7-naphthyridin-3-yl)cyclopropanecarboxamide

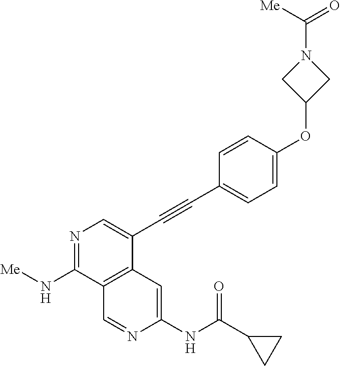 [SMILES: CNc1ncc(C#Cc2ccc(OC3CN(C(C)=O)C3)cc2)c2cc(NC(=O)C3CC3)ncc12]

N-(5-((4-((1-acetylazetidin-3-yl)oxy)phenyl)ethynyl)-8-(methylamino)-2,7-naphthyridin-3-yl)cyclopropanecarboxamide was synthesized using a similar procedure that was previously described in Example 595, by using Nr(5-ethynyl-8-(methylamino)-2,7-naphthyridin-3-yl)cyclopropanecarboxamide and 1-(3-(4-bromophenoxy)azetidin-1-yl)ethan-1-one as the starting material. LCMS (ESI) m/z 456.2, [M+H]⁺. ¹H NMR (400 MHz, DMSO-d₆) δ 11.06 (s, 1H), 9.37 (s, 1H), 8.64 (s, 1H), 8.34-8.27 (m, 1H), 8.25 (s, 1H), 7.55-7.48 (m, 2H), 6.97-6.90 (m, 2H), 5.04-5.13 (m, 1H), 4.54-4.61 (m, 1H), 4.26-4.35 (m, 1H), 4.07-4.15 (m, 1H), 3.75-3.82 (m, 1H), 3.02 (d, J=4.4 Hz, 3H), 2.04-2.14 (m, 1H), 1.80 (s, 3H), 0.95-0.79 (m, 4H).

### Example 706: Synthesis of N-(8-(methylamino)-5-((4-((3-methyloxetan-3-yl)oxy)phenyl)ethynyl)-2,7-naphthyridin-3-yl)cyclopropanecarboxamide

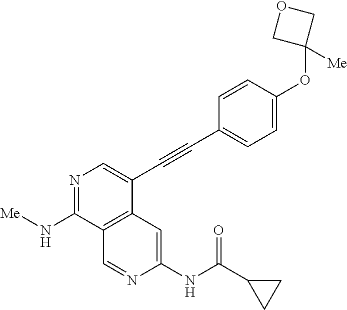 [SMILES: CNc1ncc(C#Cc2ccc(OC3(C)COC3)cc2)c2cc(NC(=O)C3CC3)ncc12]

### Step 1: diethyl 2-(4-bromophenoxy)malonate

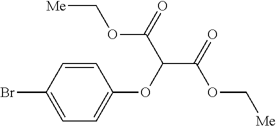 [SMILES: CCOC(=O)C(Oc1ccc(Br)cc1)C(=O)OCC]

To a stirred solution of 4-bromophenol (1.00 g; 5.780 mmol; 1.00 eq.) in MeCN (6 mL) was added diethyl 2-chloromalonate (1.13 g; 5.791 mmol; 1.01 eq.) and K₂CO₃ (2.01 g; 14.507 mmol; 2.51 eq.) under nitrogen atmosphere. The resulting solution was stirred at room temperature for 12 hours. The solvent was concentrated under vacuum. The residue was purified by flash chromatography on silica gel column using 10-20% of EtOAc in petroleum ether as eluent to provide diethyl 2-(4-bromophenoxy)malonate as a colorless solid (1.5 g, 78.4%).

### Step 2: diethyl 2-(4-bromophenoxy)-2-methylmalonate

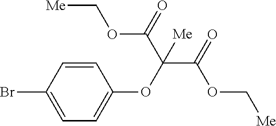 [SMILES: CCOC(=O)C(C)(Oc1ccc(Br)cc1)C(=O)OCC]

To a stirred solution of diethyl 2-(4-bromophenoxy)malonate (1.60 g; 4.831 mmol; 1.00 eq.) and Cs₂CO₃ (4.74 g; 14.551 mmol; 3.01 eq.) in DMF (15 mL) was added MeI (1.38 g; 9.694 mmol; 2.01 eq.) under nitrogen atmosphere. The resulting solution was stirred at room temperature for 12 hours. The resulting mixture was diluted with water (100 mL) extracted with EtOAc (3×100 mL). The combined organic layers were washed with brine (3×100 mL), dried over anhydrous Na₂SO₄. After filtration, the filtrate was concentrated under reduced pressure. The residue was purified by flash chromatography on silica gel column using 10-20% of EtOAc in petroleum ether as eluent to provide diethyl 2-(4-bromophenoxy)-2-methylmalonate as a colorless liquid (1.1 g, 65.9%).

### Step 3: 2-(4-bromophenoxy)-2-methylpropane-1,3-diol

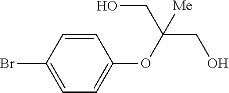 [SMILES: CC(CO)(CO)Oc1ccc(Br)cc1]

To a stirred solution of diethyl 2-(4-bromophenoxy)-2-methylmalonate (1000.0 mg; 2.896 mmol; 1.00 eq.) in MeOH (6 mL) was added NaBH₄ (330.0 mg; 8.72 mmol; 3.01 eq.) in portions at 0° C. under nitrogen atmosphere. The resulting solution was stirred at 0° C. for 1 hour. The reaction was quenched by the addition of Na₂SO₄.10H₂O at room temperature. The solids were filtered out, the filtrate was concentrated under reduced pressure. The residue was purified by flash chromatography on silica gel column using 5-10% of MeOH in CH₂Cl₂ to provide 2-(4-bromophenoxy)-2-methylpropane-1,3-diol as a white solid (800.0 mg, 88.1%). LCMS (ESI) m/z 261.0, [M+H]⁺.

### Step 4: 2-(4-bromophenoxy)-3-hydroxy-2-methylpropyl 4-methylbenzenesulfonate

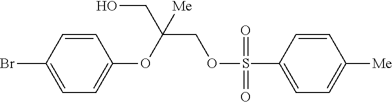 [SMILES: Cc1ccc(S(=O)(=O)OCC(C)(CO)Oc2ccc(Br)cc2)cc1]

To a solution of 2-(4-bromophenoxy)-2-methylpropane-1,3-diol (600.0 mg; 2.298 mmol; 1.00 eq.) in CH₂Cl₂ (20 mL) were added Et₃N (699.1 mg; 6.908 mmol; 3.01 eq.) and DMAP (56.2 mg; 0.458 mmol; 0.20 eq.) under nitrogen atmosphere. The resulting solution was stirred at room temperature for 1 hour. To the above solution was added TsCl (482.5 mg; 2.528 mmol; 1.10 eq.) dropwise at 0° C. The resulting solution was stirred at 0° C. for 2 hours. The mixture was diluted with CH₂Cl₂ (150 mL) and washed with brine (3×50 mL). The organic layer was concentrated under reduced pressure to afford 2-(4-bromophenoxy)-3-hydroxy-2-methylpropyl 4-methylbenzenesulfonate as a brown oil (950 mg, crude). The crude product was used in the next step directly without further purification. LCMS (ESI) m/z 415.0, [M+H]⁺.

### Step 5: 3-(4-bromophenoxy)-3-methyloxetane

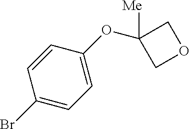 [SMILES: CC1(Oc2ccc(Br)cc2)COC1]

To a stirred solution of 2-(4-bromophenoxy)-3-hydroxy-2-methylpropyl 4-methylbenzenesulfonate (950.0 mg; 2.300 mmol; 1.00 eq.) in THF (30 mL) was added sodium hydride (60% dispersion in mineral oil, 184.1 mg; 4.600 mmol; 2.00 eq.) at 0° C. under nitrogen atmosphere. The mixture was stirred at room temperature for 2 hours. The reaction mixture was quenched by the addition of water (20 mL) and extracted with CH₂Cl₂ (3×25 mL). The organic layers were combined and concentrated under reduced pressure. The residue was purified by flash chromatography on silica gel column using 10-20% of EtOAc in petroleum ether as eluent to provide 3-(4-bromophenoxy)-3-methyloxetane as a yellow liquid (330 mg, 59.1.%).

### Step 6: N-(8-(methylamino)-5-((4-((3-methyloxetan-3-yl)oxy)phenyl)ethynyl)-2,7-naphthyridin-3-yl)cyclopropanecarboxamide

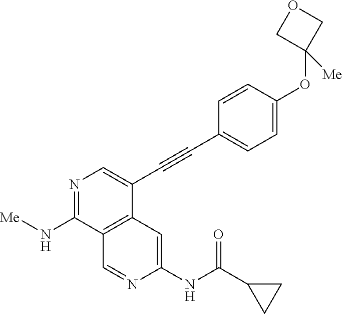 [SMILES: CNc1ncc(C#Cc2ccc(OC3(C)COC3)cc2)c2cc(NC(=O)C3CC3)ncc12]

N-(8-(methylamino)-5-((4-((3-methyloxetan-3-yl)oxy)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-(4-bromophenoxy)-3-methyloxetane as the starting material. LCMS (ESI) m/z 429.2, [M+H]⁺. ¹H NMR (400 MHz, DMSO-d₆) δ 11.06 (s, 1H), 9.37 (s, 1H), 8.62 (s, 1H), 8.31-8.27 (m, 1H), 8.25 (s, 1H), 7.47 (d, J=8.4 Hz, 2H), 6.76 (d, J=8.4 Hz, 2H), 4.77 (d, J=6.8 Hz, 2H), 4.62 (d, J=6.8 Hz, 2H), 3.02 (d, J=4.4 Hz, 3H), 2.11-2.05 (m, 1H), 1.69 (s, 3H), 0.91-0.80 (m, 4H).

### Example 707: Synthesis of N-(8-(methylamino)-5-((4-(oxetan-3-yloxy)phenyl)ethynyl)-2,7-naphthyridin-3-yl)cyclopropanecarboxamide

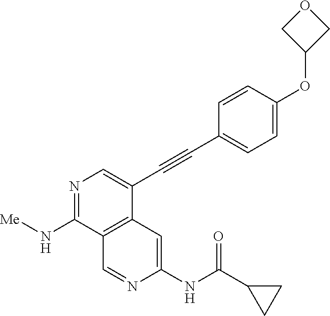 [SMILES: CNc1ncc(C#Cc2ccc(OC3COC3)cc2)c2cc(NC(=O)C3CC3)ncc12]

### Step 1: 3-(4-bromophenoxy)oxetane

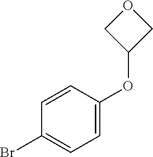 [SMILES: Brc1ccc(OC2COC2)cc1]

To a stirred solution of 4-bromophenol (500.0 mg; 2.890 mmol; 1.00 eq.) and Cs₂CO₃ (2.83 g; 8.698 mmol; 3.01 eq.) in DMF (5 mL) was added 3-bromooxetane (790.0 mg; 5.767 mmol; 2.00 eq.) under nitrogen atmosphere. The resulting solution was stirred at 50° C. for 2 hours. The resulting mixture was diluted with water (30 mL) and extracted with EtOAc (3×30 mL). The combined organic layers were washed by brine (3×10 mL), dried over anhydrous Na₂SO₄. After filtration, the filtrate was concentrated under reduced pressure. The residue was purified by flash chromatography on silica gel column using 20-30% of EtOAc in petroleum ether as eluent to provide 3-(4-bromophenoxy)oxetane as a yellow solid (400.0 mg, 60.4%).

### Step 2: N-(8-(methylamino)-5-((4-(oxetan-3-yloxy)phenyl)ethynyl)-2,7-naphthyridin-3-yl)cyclopropanecarboxamide

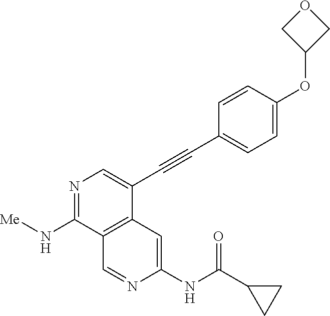 [SMILES: CNc1ncc(C#Cc2ccc(OC3COC3)cc2)c2cc(NC(=O)C3CC3)ncc12]

N-(8-(methylamino)-5-((4-(oxetan-3-yloxy)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-(4-bromophenoxy)oxetane as the starting material. LCMS (ESI) m/z 415.2, [M+H]⁺. ¹H NMR. (400 MHz, DMSO-d₆) δ 11.06 (s, 1H), 9.36 (s, 1H), 8.63 (s, 1H), 8.31-8.27 (m, 1H), 8.25 (s, 1H), 7.53-7.44 (m, 2H), 6.91-6.83 (m, 2H), 5.39-5.31 (m, 1H), 5.03-4.95 (m, 2H), 4.65-4.55 (m, 2H), 3.02 (d, J=4.4 Hz, 3H), 2.13-2.04 (m, 1H), 0.92-0.82 (m, 4H).

### Example 708: Synthesis of (R)—N-(8-(methylamino)-5-((4-(2-methylmorpholino)phenyl)ethynyl)-2,7-naphthyridin-3-yl)cyclopropanecarboxamide

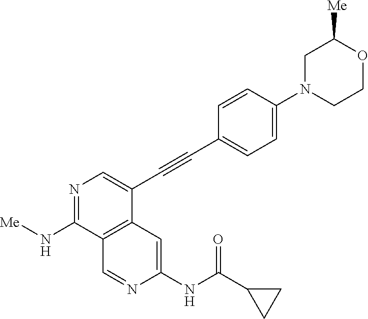 [SMILES: CNc1ncc(C#Cc2ccc(N3CCO[C@H](C)C3)cc2)c2cc(NC(=O)C3CC3)ncc12]

### Step 1: (R)-4-(4-bromophenyl)-2-methylmorpholine

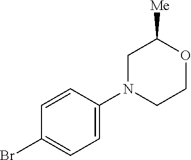 [SMILES: C[C@@H]1CN(c2ccc(Br)cc2)CCO1]

To a stirred solution of 1,4-dibromobenzene (233.2 mg; 0.988 mmol; 2.00 eq.) and (8)-2-methylmorpholine (50.0 mg; 0.494 mmol; 1.00 eq.) in toluene (1 mL) were added Pd₂(dba)₃ (45.3 mg; 0.049 mmol; 0.10 eq.), BINAP (61.6 mg; 0.099 mmol; 0.20 eq.), t-BuONa (66.5 mg; 0.692 mmol; 1.40 eq.) under nitrogen atmosphere. The resulting mixture was stirred at 80° C. for 14 hours. The desired product was detected via LCMS. The mixture was allowed to cool down to room temperature. The resulting mixture was concentrated under reduced pressure. The residue was purified by flash chromatography on silica gel column using 10-30% of EtOAc in petroleum ether as eluent to provide (R)-4-(4-bromophenyl)-2-methylmorpholine as a light-yellow solid (100.0 mg, 79.1%). LCMS (ESI) m/z 256.0, [M+H]⁺.

### Step 2: (R)—N-(8-(methylamino)-5-((4-(2-methylmorpholino)phenyl)ethynyl)-2,7-naphthyridin-3-yl)cyclopropanecarboxamide

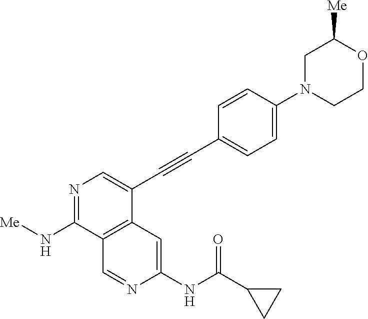 [SMILES: CNc1ncc(C#Cc2ccc(N3CCO[C@H](C)C3)cc2)c2cc(NC(=O)C3CC3)ncc12]

(R)—N-(8-(methylamino)-5-((4-(2-methylmorpholino)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and (R)-4-(4-bromophenyl)-2-methylmorpholine as the starting material. LCMS (ESI) m/z 442.2, [M+H]⁺. ¹H NMR (300 MHz, DMSO-d₆) δ 11.04 (s, 1H), 9.36 (s, 1H), 8.65 (s, 1H), 8.27-8.18 (m, 2H), 7.47-7.36 (m, 2H), 7.04-6.93 (m, 2H), 3.98-3.86 (m, 1H), 3.74-3.54 (m, 4H), 3.02 (d, J=4.4 Hz, 3H), 2.78-2.63 (m, 1H), 2.44-2.35 (m, 1H), 2.16-2.02 (m, 1H), 1.17 (d, J=6.1 Hz, 3H), 0.96-0.78 (m, 4H).

### Example 709: Synthesis of (S)—N-(8-(methylamino)-5-((4-(2-methylmorpholino)phenyl)ethynyl)-2,7-naphthyridin-3-yl)cyclopropanecarboxamide

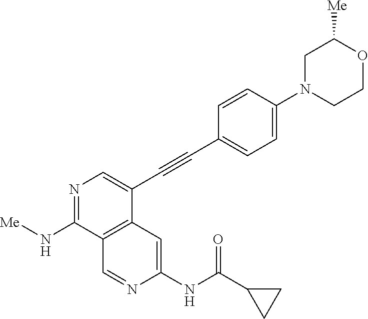 [SMILES: CNc1ncc(C#Cc2ccc(N3CCO[C@@H](C)C3)cc2)c2cc(NC(=O)C3CC3)ncc12]

### Step 1: (S)-4-(4-bromophenyl)-2-methylmorpholine

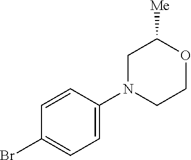 [SMILES: C[C@H]1CN(c2ccc(Br)cc2)CCO1]

To a stirred solution of 1,4-dibromobenzene (233.2 mg; 0.988 mmol; 2.00 eq.) and (S)-2-methylmorpholine (50.0 mg; 0.494 mmol; 1.00 eq.) in toluene (1 mL) were added Pd₂(dba)₃ (45.3 mg; 0.049 mmol; 0.10 eq.), BINAP (61.6 mg; 0.099 mmol; 0.20 eq.) t-BuONa (66.5 mg; 0.692 mmol; 1.40 eq.) under nitrogen atmosphere. The resulting mixture was stirred at 80° C. for 14 hours. The desired product was detected via LCMS. The mixture was allowed to cool down to room temperature. The solvent was concentrated under reduced pressure. The residue was purified by flash chromatography on silica gel column using 10-30% of EtOAc in petroleum ether as eluent to provide (S)-4-(4-bromophenyl)-2-methylmorpholine as a light-yellow solid (105.0 mg, 83.0%). LCMS (ESI) m/z 256.0, [M+H]⁺.

### Step 2: .S)—N-(8-(methylamino)-5-((4-(2-methylmorpholino)phenyl)ethynyl)-2,7-naphthyridin-3-yl)cyclopropanecarboxamide

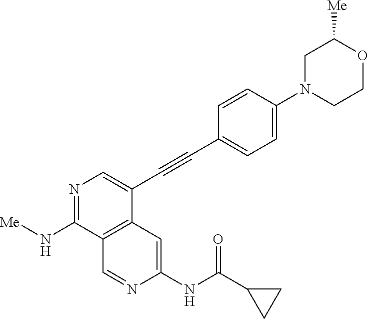 [SMILES: CNc1ncc(C#Cc2ccc(N3CCO[C@@H](C)C3)cc2)c2cc(NC(=O)C3CC3)ncc12]

(S)—N-(8-(methylamino)-5-((4-(2-methylmorpholino)phenyl)ethynyl)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and (S)-4-(4-bromophenyl)-2-methylmorpholine as the starting material. LCMS (ESI) m/z 442.2, [M+H]⁺. ¹H NMR (400 MHz, DMSO-d₆) δ 11.05 (s, 1H), 9.36 (s, 1H), 8.65 (s, 1H), 8.30-8.23 (m, 1H), 8.22 (s, 1H), 7.45-7.38 (m, 2H), 7.04-6.96 (m, 2H), 3.96-3.92 (m, 1H), 3.75-3.56 (m, 4H), 3.01 (d, J=4.4 Hz, 3H), 2.76-2.65 (m, 1H), 2.44-2.31 (m, 1H), 2.10-2.06 (m, 1H), 1.17 (d, J=6.2 Hz, 3H), 0.93-0.80 (m, 4H).

### Example 710: Synthesis of N-(5-((4-(2-cyano-2-methylpropoxy)phenyl)ethynyl)-8-(methylamino)-2,7-naphthyridin-3-yl)cyclopropanecarboxamide

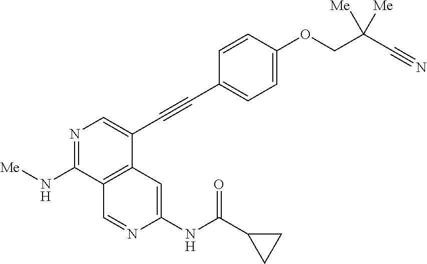 [SMILES: CNc1ncc(C#Cc2ccc(OCC(C)(C)C#N)cc2)c2cc(NC(=O)C3CC3)ncc12]

### Step 1: 2-cyano-2-methylpropyl 4-methylbenzenesulfonate

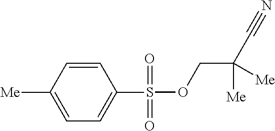 [SMILES: Cc1ccc(S(=O)(=O)OCC(C)(C)C#N)cc1]

To a solution of 3-hydroxy-2,2-dimethylpropanenitrile (200.0 mg; 2.017 mmol; 1.00 eq.), Et₃N (612.4 mg; 6.051 mmol; 3.00 eq.) and DMAP (49.3 mg; 0.403 mmol; 0.20 eq.) in CH₂Cl₂ (10 mL) was added 4-methylbenzenesulfonyl chloride (423.0 mg; 2.219 mmol; 1.1.0 eq.) at 0° C. The reaction was stirred at room temperature for 3 hours. The desired product was detected via LCMS. The reaction was quenched with water. The resulting mixture was diluted with CH₂Cl₂ (50 ml) and washed with saturated NH₄HCO₃ solution (3×10 mL). The organic layer was dried over anhydrous sodium sulfate. After filtration, the filtrate was concentrated under reduced pressure to afford 2-cyano-2-methylpropyl 4-methylbenzenesulfonate as a white oil (579.0 mg, crude). LCMS (ESI) m/z 254.1, [M+H]⁺.

### Step 2: 3-(4-bromophenoxy)-2,2-dimethylpropanenitrile

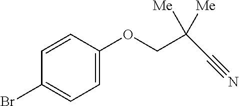 [SMILES: CC(C)(C#N)COc1ccc(Br)cc1]

A solution of 2-cyano-2-methylpropyl 4-methylbenzenesulfonate (579.0 mg; 2.286 mmol; 2.00 eq.), 4-bromophenol (197.0 mg; 1.139 mmol; 1.00 eq.) and Cs₂CO₃ (1.11 g; 3.407 mmol; 3.00 eq.) in DMF (4 mL) was stirred at 50° C. for 2 hours under nitrogen atmosphere. The desired product was detected via LCMS. The solvent was concentrated under vacuum. The residue was purified by flash chromatography on silica gel column using 10-50% of EtOAc in petroleum ether as eluent to provide 3-(4-bromophenoxy)-2,2-dimethylpropanenitrile as a white oil (116.0 mg, 19.9%). LCMS (ESI) m/z 254.0, [M+H]⁺.

### Step 3: N-(5-((4-(2-cyano-2-methylpropoxy)phenyl)ethynyl)-8-(methylamino)-2,7-naphthyridin-3-yl)cyclopropanecarboxamide

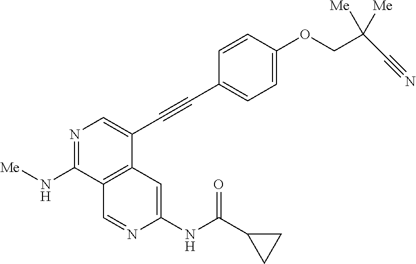 [SMILES: CNc1ncc(C#Cc2ccc(OCC(C)(C)C#N)cc2)c2cc(NC(=O)C3CC3)ncc12]

N-(5-((4-(2-cyano-2-methylpropoxy)phenyl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-(4-bromophenoxy)-2,2-dimethylpropanenitrile as the starting material. LCMS (ESI) m/z 440.2, [M+H]⁺. ¹H NMR (400 MHz, DMSO-d₆) δ 11.06 (s, 1H), 9.37 (s, 1H), 8.66 (s, 1H), 8.31-8.26 (m, 1H), 8.25 (s, 1H), 7.57-7.49 (m, 2H), 7.12-7.04 (m, 2H), 4.09 (s, 2H), 3.02 (d, J=4.4 Hz, 3H), 2.12-2.04 (m, 1H), 1.44 (s, 6H), 0.94-0.80 (m, 4H).

### Example 711: Synthesis of N-(5-((4-((1-methyl-1H-1,2,4-triazol-3-yl)oxy)phenyl)ethynyl)-8-(methylamino)-2,7-naphthyridin-3-yl)cyclopropanecarboxamide

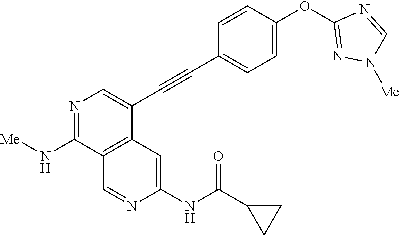 [SMILES: CNc1ncc(C#Cc2ccc(Oc3ncn(C)n3)cc2)c2cc(NC(=O)C3CC3)ncc12]

### Step 1: 3-(4-bromophenoxy)-1-methyl-1H-1,2,4-triazole

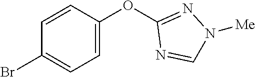 [SMILES: Cn1cnc(Oc2ccc(Br)cc2)n1]

A solution of 4-bromophenol (500.0 mg; 2.891 mmol; 1.00 eq.), 3-bromo-1-methyl-1H-1,2,4-triazole (936.0 mg; 5.782 mmol; 2.00 eq.) and t-BuOK (388.1 mg; 3.469 mmol; 1.20 eq.) in DMSO (7 mL) was stirred at 180° C. for 1 hour under microwave radiation. The desired product was detected via LCMS. The resulting mixture was diluted with EtOAc (100 mL) and washed with brine (3×20 mL). The organic layer was dried over anhydrous Na₂SO₄. After filtration, the filtrate was concentrated under reduced pressure. The residue was purified by flash chromatography on pre-packed C18 column using 0-40% of MeCN in water (10 mmol/L NH₄HCO₃) as eluent to provide 3-(4-bromophenoxy)-1-methyl-1H-1,2,4-triazole as brown oil (204.1 mg, 1.9.0%). LCMS (ESI) m/z 254.0, [M+H]⁺.

### Step 2: N-(5-((4-((1-methyl-1H-1,2,4-triazol-3-yl)oxy)phenyl)ethynyl)-8-(methylamino)-2,7-naphthyridin-3-yl)cyclopropanecarboxamide

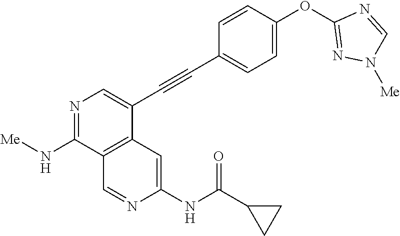 [SMILES: CNc1ncc(C#Cc2ccc(Oc3ncn(C)n3)cc2)c2cc(NC(=O)C3CC3)ncc12]

N-(5-((4-((1-methyl-1H-1,2,4-triazol 3-yl)oxy)phenyl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-(4-bromophenoxy)-1-methyl-1H-1,2,4-triazole as the starting material. LCMS (ESI) m/z 440.2, [M+H]⁺. ¹H NMR (400 MHz, DMSO-d₆) δ 11.06 (s, 1H), 9.37 (s, 1H), 8.65 (s, 1H), 8.36 (s, 1H), 8.35-8.30 (m, 1H), 8.26 (s, 1H), 7.62-7.54 (m, 2H), 7.29-7.21 (m, 2H), 3.81 (s, 3H), 3.03 (d, J=4.4 Hz, 3H), 2.13-2.04 (m, 1H), 0.97-0.83 (m, 4H).

### Example 712: Synthesis of N-(8-(methylamino)-5-((4-((1-methylazetidin-3-yl)methoxy)phenyl)ethynyl)-2,7-naphthyridin-3-yl)cyclopropanecarboxamide

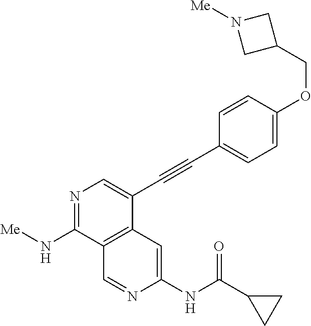 [SMILES: CNc1ncc(C#Cc2ccc(OCC3CN(C)C3)cc2)c2cc(NC(=O)C3CC3)ncc12]

### Step 1: tert-butyl 3-((4-bromophenoxy)methyl)azetidine-1-carboxylate

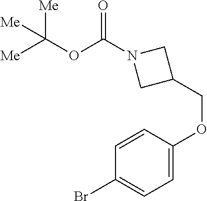 [SMILES: CC(C)(C)OC(=O)N1CC(COc2ccc(Br)cc2)C1]

A solution of tert-butyl 3-(bromomethyl)azetidine-1-carboxylate (1.08 g; 4.335 mmol; 1.50 eq.), 4-bromophenol (500.0 mg; 2.890 mmol; 1.00 eq.) and Cs₂CO₃ (1.88 g; 5.780 mmol; 2.00 eq.) in DMF (10.0 mL) was stirred at 50° C. for 1 hour. The desired product was detected via LCMS. The reaction mixture was added dropwise into 150 mL water. The precipitated solids were collected by filtration and washed with water (3×3 mL) to provide tert-butyl 3-((4-bromophenoxy)methyl)azetidine-1-carboxylate as a white solid (908.0 mg, 89.6%). LCMS (ESI) m/z 342.1, [M+H]⁺.

### Step 2: 3-((4-bromophenoxy)methyl)azetidine hydrochloride

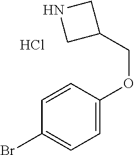 [SMILES: Brc1ccc(OCC2CNC2)cc1 ; Cl]

To a solution of tert-butyl 3-(4-bromophenoxymethyl)azetidine-1-carboxylate (400.0 mg; 1.169 mmol; 1.00 eq.) in MeOH (2.0 mL) was added HCl (gas) (4 M in 1,4-dioxane; 6.0 mL). The reaction mixture was stirred at room temperature for 1 hour. The desired product was detected via LCMS. The resulting mixture was concentrated under reduced pressure to provide 3-((4-bromophenoxy)methyl)azetidine hydrochloride as a white solid (327.5 mg, 85.2%). LCMS (ESI) m/z 242.0, [M+H]⁺.

### Step 3: 3-((4-bromophenoxy)methyl)-1-methylazetidine

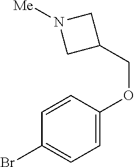 [SMILES: CN1CC(COc2ccc(Br)cc2)C1]

A solution of 3-(4-bromophenoxymethyl)azetidine hydrochloride (327.9 mg; 1.177 mmol; 1.00 eq.) and formaldehyde (37% w/w in water) (1.14 g; 14.124 mmol; 12.00 eq.) in CH₂Cl₂ (5 mL) was stirred at room temperature for 4 hours. To the above solution was added NaBH(OAc)₃ (2.99 g; 14.124 mmol; 12.00 eq.). The resulting solution was stirred at room temperature for 1 hour. The desired product was detected via LCMS. The mixture was diluted with water (30 mL) and neutralized to pH=8 with saturated NaHCO₃ solution. The mixture was extracted with EtOAc (3×15 mL). The organic layer was dried over anhydrous Na₂SO₄. After filtration, the filtrate was concentrated under reduced pressure to provide 3-((4-bromophenoxy)methyl)-1-methylazetidine as a yellow oil (258.7 mg, 83.1%). LCMS (ESI) m/z 256.0, [M+H]⁺.

### Step 4: N-(8-(methylamino)-5-((4-((1-methylazetidin-3-yl)methoxy)phenyl)ethynyl)-2,7-naphthyridin-3-yl)cyclopropanecarboxamide

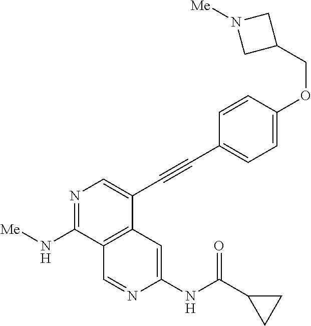 [SMILES: CNc1ncc(C#Cc2ccc(OCC3CN(C)C3)cc2)c2cc(NC(=O)C3CC3)ncc12]

N-(8-(methyl amino)-5-((4-((1-methylazetidin-3-yl)methoxy)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-((4-bromophenoxy)methyl)-1-methylazetidine as the starting material. LCMS (ESI) m/z 442.2, [M+H]⁺. ¹H NMR (400 MHz, DMSO-d₆) δ 11.05 (s, 1H), 9.37 (s, 1H), 8.64 (s, 1H), 8.28-8.24 (m, 2H), 7.49 (d, J=8.4 Hz, 2H), 7.02 (d, J=8.4 Hz, 2H), 4.15 (d, J=6.8 Hz, 2H), 3.49-3.40 (m, 2H), 3.20-3.08 (m, 2H), 3.02 (d, J=4.4 Hz, 3H), 2.85-2.79 (m, 1H), 2.31 (s, 3H), 2.11-2.06 (m, 1H), 0.90-0.84 (m, 4H).

### Example 713: Synthesis of N-(8-(methylamino)-5-((4-((1-(oxetan-3-yl)propan-2-yl)oxy)phenyl)ethynyl)-2,7-naphthyridin-3-yl)cyclopropanecarboxamide

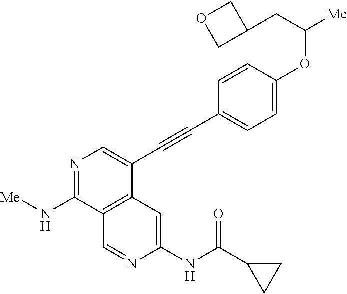 [SMILES: CNc1ncc(C#Cc2ccc(OC(C)CC3COC3)cc2)c2cc(NC(=O)C3CC3)ncc12]

### Step 1: 1-(oxetan-3-yl)propan-2-ol

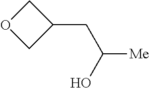

To a solution of 1-(oxetan-3-yl)propan-2-one (300.0 mg; 2.628 mmol; 1.00 eq.) in MeOH (18 mL) at 0° C. was added NaBH₄ (996.0 mg; 26.328 mmol; 10.02 eq.) in portions under nitrogen atmosphere. The reaction was stirred at room temperature for 3 hours. The reaction was quenched by the addition of saturated NH₄Cl solution at 0° C. The resulting mixture was extracted with EtOAc (3×30 mL). The combined organic layers were dried over anhydrous Na₂SO₄. After filtration, the filtrate was concentrated under reduced pressure to afford 1-(oxetan-3-yl)propan-2-ol as light yellow oil (220.0 mg, 72.0%).

### Step 2: 3-(2-(4-bromophenoxy)propyl)oxetane

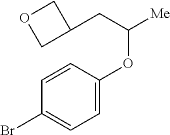 [SMILES: CC(CC1COC1)Oc1ccc(Br)cc1]

To a solution of 4-bromocyclohexan-1-ol (298.0 mg; 1.664 mmol; 1.00 eq.) and 1-(oxetan-3-yl)propan-2-ol (200.0 mg; 1.722 mmol; 1.03 eq.) in THF (12 mL) was added PPh₃ (496.0 mg; 1.891 mmol; 1.14 eq.) and DIAD (348.0 mg; 1.721 mmol; 1.03 eq.) under nitrogen atmosphere. The reaction was stirred at 50° C. for 14 hours. The desired product was detected via LCMS. The resulting mixture was concentrated under reduced pressure. The residue was purified by flash chromatography on silica gel column using 10-50% of EtOAc in petroleum ether as eluent to provide 3-(2-(4-bromophenoxy)propyl)oxetane as a light yellow oil (225.0 mg, 49.8%). LCMS (ESI) m/z 271.0, [M+H]⁺.

### Step 3: NV (8-(methylamino)-5-((4-((1-(oxetan-3-yl)propan-2-yl)oxy)phenyl)ethynyl)-2,7-naphthyridin-3-yl)cyclopropanecarboxamide

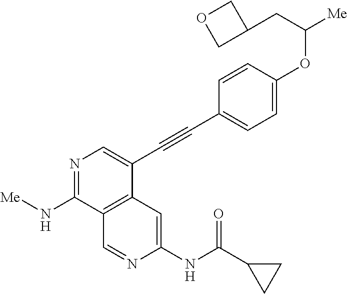 [SMILES: CNc1ncc(C#Cc2ccc(OC(C)CC3COC3)cc2)c2cc(NC(=O)C3CC3)ncc12]

N-(8-(methylamino)-5-((4-((1-(oxetan-3-yl)propan-2-yl)oxy)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-(2-(4-bromophenoxy)propyl)oxetane as the starting material. LCMS (ESI) m/z 457.1, [M+H]⁺. ¹H NMR (400 MHz, DMSO-d₆) δ 11.06 (s, 1H), 9.37 (s, 1H), 8.63 (s, 1H), 8.31-8.25 (m, 11), 8.24 (s, 1H), 7.51-7.43 (m, 2H), 7.00-6.92 (m, 2H), 4.67-4.59 (m, 2H), 4.59-4.47 (m, 1H), 4.45-4.28 (m, 2H), 3.20-3.10 (m, 1H), 3.02 (d, J=4.4 Hz, 3H), 2.1.0-1.94 (m, 3H), 1.22 (d, J=6.1 Hz, 3H), 0.93-0.80 (m, 4H).

### Example 714: Synthesis of N-(5-((4-(2-hydroxy-2-methylpropoxy)phenyl)ethynyl)-8-(methylamino)-2,7-naphthyridin-3-yl)cyclopropanecarboxamide

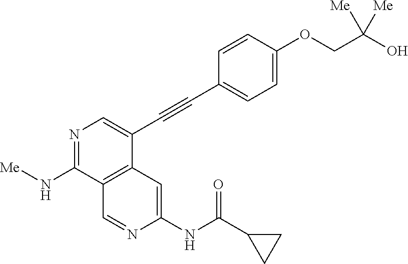 [SMILES: CNc1ncc(C#Cc2ccc(OCC(C)(C)O)cc2)c2cc(NC(=O)C3CC3)ncc12]

### Step 1: 1-(4-bromophenoxy)-2-methylpropan-2-ol

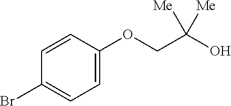 [SMILES: CC(C)(O)COc1ccc(Br)cc1]

To a solution of 4-bromophenol (500.1 mg; 2.891 mmol; 1.00 eq.) and Cs₂CO₃ (1.90 g; 5.782 mmol; 2.00 eq.) in DMF (10 mL) was added 1-bromo-2-methylpropan-2-ol (486.2 mg; 3.170 mmol; 1.10 eq.). The resulting solution was stirred at 100° C. for 2 hours under nitrogen atmosphere. The resulting mixture was diluted with EtOAc (100 mL) and washed with brine (3×20 mL). The organic layer was dried over anhydrous Na₂SO₄. After filtration, the filtrate was concentrated under reduced pressure. The residue was purified by flash chromatography on silica gel column using 10-30% of EtOAc in petroleum ether as eluent to provide 1-(4-bromophenoxy)-2-methylpropan-2-ol as an off-white oil (220.1 mg, 30.2%).

### Step 2: N-(5-((4-(2-hydroxy-2-methylpropoxy)phenyl)ethynyl)-8-(methylamino)-2,7-naphthyridin-3-yl)cyclopropanecarboxamide

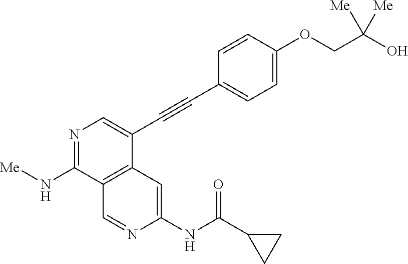 [SMILES: CNc1ncc(C#Cc2ccc(OCC(C)(C)O)cc2)c2cc(NC(=O)C3CC3)ncc12]

N-(5-((4-(2-hydroxy-2-methylpropoxy)phenyl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 1-(4-bromophenoxy)-2-methylpropan-2-ol as the starting material. LCMS (ESI) m/z 431.2, [M+H]⁺. ¹H NMR (400 MHz, DMSO-d₆) δ 11.07 (s, 1H), 9.37 (s, 1H), 8.64 (s, 1H), 8.31-8.27 (m, 1H), 8.25 (s, 1H), 7.49 (d, J=8.8 Hz, 2H), 7.01 (d, J=8.8 Hz, 2H), 4.65 (s, 1H), 3.77 (s, 2H), 3.02 (d, J=4.4 Hz, 3H), 2.13-2.02 (m, 1H), 1.23 (s, 6H), 0.95-0.81 (m, 4H).

### Example 715: Synthesis of N-(8-(methylamino)-5-((4-((1-(methylsulfonyl)azetidin-3-yl)oxy)phenyl)ethynyl)-2,7-naphthyridin-3-yl)cyclopropanecarboxamide

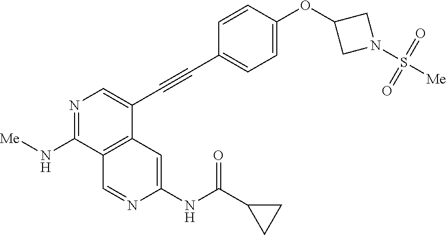 [SMILES: CNc1ncc(C#Cc2ccc(OC3CN(S(C)(=O)=O)C3)cc2)c2cc(NC(=O)C3CC3)ncc12]

### Step 1: tert-butyl 3-(4-bromophenoxy)azetidine-1-carboxylate

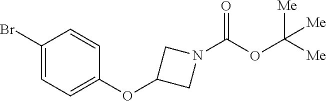 [SMILES: CC(C)(C)OC(=O)N1CC(Oc2ccc(Br)cc2)C1]

A solution of 4-bromophenol (500.0 mg; 2.890 mmol; 1.00 eq.), tert-butyl 3-bromoazetidine-1-carboxylate (1.02 g; 4.335 mmol; 1.50 eq.) and Cs₂CO₃ (2.80 g; 8.670 mmol; 3.00 eq.) in DMF (10.0 mL) was stirred at 1.20° C. for 1 hour. The desired product was detected via LCMS. The resulting mixture was diluted with EtOAc (60 mL) and washed with brine (3×5 ML), The organic layer was concentrated under vacuum. The residue was purified by flash chromatography on pre-packed C18 column using 50-80% of MeOH in water (10 mmol/L NH₄HCO₃) as eluent to provide tert-butyl 3-(4-bromophenoxy)azetidine-1-carboxylate as a yellow solid (1.35 g, 67.7%). LCMS (ESI) m/z 328.1, [M+H]⁺.

### Step 2: 3-(4-bromophenoxy)azetidine hydrochloride

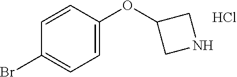 [SMILES: Brc1ccc(OC2CNC2)cc1 ; Cl]

A solution of tert-butyl 3-(4-bromophenoxy)azetidine-1-carboxylate (500.0 mg; 1.523 mmol; 1.00 eq.) in HCl (gas) (4 M in MeOH, 5.0 mL) was stirred at room temperature for 1 hour. The desired product was detected via LCMS. The resulting mixture was concentrated under reduced pressure to provide 3-(4-bromophenoxy)azetidine hydrochloride as a white solid (454.2 mg, 67.6%). LCMS (ESI) m/z 264.0, [M+H]⁺.

### Step 3: 3-(4-bromophenoxy)-1-(methylsulfonyl)azetidine

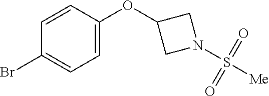 [SMILES: CS(=O)(=O)N1CC(Oc2ccc(Br)cc2)C1]

To a stirred solution of 3-(4-bromophenoxy)azetidine hydrochloride (400.0 mg; 1.512 mmol; 1.00 eq.) and Et₃N (459.0 mg; 4.536 mmol; 3.00 eq.) in CH₂Cl₂ (5.0 mL) was added methanesulfonic anhydride (395.0 mg; 2.268 mmol; 1.50 eq.). The reaction mixture was stirred at room temperature for 1 hour. The desired product was detected via LCMS. The resulting mixture was concentrated under vacuum. The residue was purified by flash chromatography on silica gel column using 40-50% of EtOAc in petroleum ether as eluent to provide3-(4-bromophenoxy)-1-(methylsulfonyl)azetidine as a yellow solid (427.8 mg, 87.1%). LCMS (ESI) m/z 306.0, [M+H]⁺.

### Step 4: N-(8-(methylamino)-5-((4-((1-(methylsulfonyl)azetidin-3-yl)oxy)phenyl)ethynyl)-2,7-naphthyridin-3-yl)cyclopropanecarboxamide

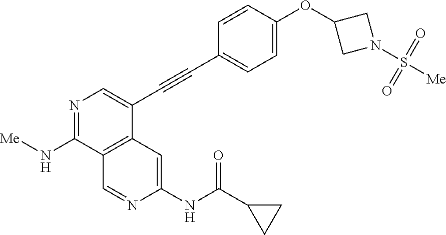 [SMILES: CNc1ncc(C#Cc2ccc(OC3CN(S(C)(=O)=O)C3)cc2)c2cc(NC(=O)C3CC3)ncc12]

N-(8-(methylamino)-5-((4-((1-(methylsulfonyl)azetidin-3-yl)oxy)phenyl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-(4-bromophenoxy)-1-(methylsulfonyl)azetidine as the starting material. LCMS (ESI) m/z 492.2, [M+H]⁺. ¹H NMR (400 MHz, DMSO-d₆) δ 11.07 (s, 1H), 9.37 (s, 1H), 8.64 (s, 1H), 8.31-8.25 (m, 2H), 7.53-7.49 (m, 2H), 6.97-6.94 (m, 2H), 5.13-5.07 (m, 1H), 4.36-4.32 (m, 2H), 3.97-3.93 (m, 2H), 3.09 (s, 3H), 3.02 (d, J=4.4 Hz, 3H), 2.09-2.07 (m, 1H), 0.90-0.83 (m, 4H).

### Example 716: Synthesis of N-(5-((2-(methoxymethyl)-1-methyl-1H-indol-6-yl)ethynyl)-8-(methylamino)-2,7-naphthyridin-3-yl)cyclopropanecarboxamide

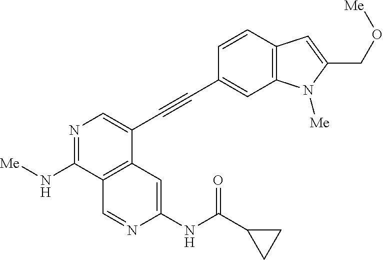 [SMILES: CNc1ncc(C#Cc2ccc3cc(COC)n(C)c3c2)c2cc(NC(=O)C3CC3)ncc12]

### Step 1: (6-bromo-1-methyl-1H-indol-2-yl)methanol

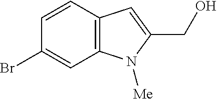 [SMILES: Cn1c(CO)cc2ccc(Br)cc21]

To a solution of methyl 6-bromo-1-methyl-1H-indole-2-carboxylate (300.0 mg; 1.119 mmol; 1.00 eq.) in THF (5 mL) was added DIBAL-H (319.8 mg; 2.249 mmol; 2.01 eq.) at 0° C. The reaction was stirred at room temperature for 1 hour. The desired product was detected via LCMS. The reaction was quenched by the addition of MeOH at 0° C. The solvent was concentrated under reduced pressure. The residue was purified by flash chromatography on silica gel column using 25-35% of EtOAc in petroleum ether as eluent to provide (6-bromo-1-methyl-1H-indol-2-yl)methanol as off-white solid (160.0 mg, 58.9%). LCMS (ESI) m/z 240.0, [M+H]⁺.

### Step 2: 6-bromo-2-(methoxymethyl)-1-methyl-1H-indole

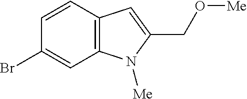 [SMILES: COCc1cc2ccc(Br)cc2n1C]

To a solution of (6-bromo-1-methyl-1H-indol-2-yl)methanol (160.0 mg; 0.666 mmol; 1.00 eq.) in THF (3 mL) was added NaH (60% dispersion in mineral oil, 132.7 mg; 3.317 mmol; 4.98 eq.) at 0° C. under nitrogen atmosphere. The reaction was stirred at room temperature for 0.5 hour. To the above solution was added methyl iodide (189.1 mg; 1.332 mmol; 2.00 eq.) at 0° C. The reaction was stirred at room temperature for 12 hours. The desired product was detected via LCMS. The reaction was quenched by the addition of saturated NH₄Cl solution at 0′C. The resulting mixture was diluted with EtOAc (50 mL) and washed with brine (2×20 mL). The organic layer was dried was dried anhydrous Na₂SO₄. After filtration, the filtrate was concentrated under reduced pressure. The residue was purified by flash chromatography on silica gel column using 20-35% of EtOAc in petroleum ether as eluent to provide 6-bromo-2-(methoxymethyl)-1-methyl-1H-indole as off-white solid (90.0 mg, 52.1%). LCMS (ESI) m/z 254.0, [M+H]⁺.

### Step 3: N-(5-((2-(methoxymethyl)-1-methyl-1H-indol-6-yl)ethynyl)-8-(methylamino)-2,7-naphthyridin-3-yl)cyclopropanecarboxamide

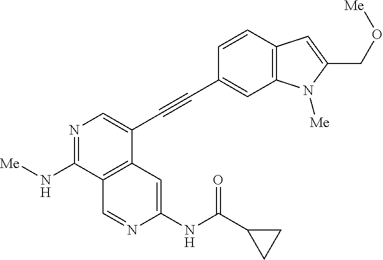 [SMILES: CNc1ncc(C#Cc2ccc3cc(COC)n(C)c3c2)c2cc(NC(=O)C3CC3)ncc12]

N-(5-((2-(methoxymethyl)-1-methyl-1-1-indol-6-yl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 6-bromo-2-(methoxymethyl)-1-methyl-1H-indole as the starting material. LCMS (ESI) m/z 440.2, [M+H]⁺. ¹H NMR (400 MHz, DMSO-d₆) δ 11.11 (s, 1H), 9.38 (s, 1H), 8.83 (s, 1H), 8.31-8.27 (m, 1H), 8.26 (s, 1H), 7.82 (s, 1H), 7.56 (d, J=8.0 Hz, 1H), 7.20 (dd, J=8.0, 1.2 Hz, 1H), 6.52 (s, 1H), 4.62 (s, 2H), 3.79 (s, 3H), 3.31 (s, 3H), 3.02 (d, J=4.4 Hz, 3H), 2.14-2.08 (m, 1H), 0.94-0.82 (m, 4H).

### Example 717: Synthesis of N-(5-((2-(methoxymethyl)benzofuran-5-yl)ethynyl)-8-(methylamino)-2,7-naphthyridin-3-yl)cyclopropanecarboxamide

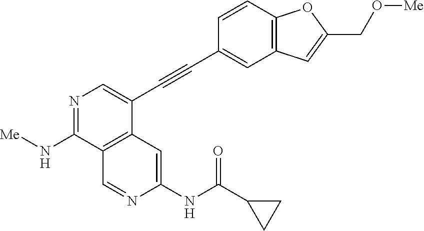 [SMILES: CNc1ncc(C#Cc2ccc3oc(COC)cc3c2)c2cc(NC(=O)C3CC3)ncc12]

### Step 1: (5-bromobenzofuran-2-yl)methanol

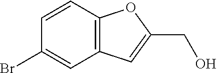 [SMILES: OCc1cc2cc(Br)ccc2o1]

To a stirred solution of ethyl 5-bromobenzofuran-2-carboxylate (500.0 mg; 1.858 mmol; 1.00 eq.) in MeOH (15.0 mL) was added NaBH₄ (702.9 mg; 18.580 mmol; 10.00 eq.) in portions at 0° C. under nitrogen atmosphere. The reaction mixture was stirred at room temperature for 14 hours. The desired product was detected via LCMS. The resulting mixture was diluted with EtOAc (50 mL) and washed with brine (3×5 mL). The organic layer was concentrated under vacuum. The residue was purified by flash chromatography on silica gel column using 30-50% of EtOAc in petroleum ether as eluent to provide (5-bromobenzofuran-2-yl)methanol as a white solid (323.4 mg, 76.5%). LCMS (ESI) m/z 227.0, [M+H]⁺.

### Step 2: 5-bromo-2-(methoxymethyl)benzofuran

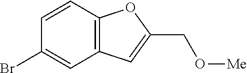 [SMILES: COCc1cc2cc(Br)ccc2o1]

To a stirred solution of (5-bromobenzofuran-2-yl)methanol (429.0 mg; 1.889 mmol; 1.00 eq.) in THF (1.0.0 mL) was added NaH (60% dispersion in mineral oil, 11.3.3 mg; 2.833 mmol; 1.50 eq.) at 0° C. under nitrogen atmosphere. The reaction mixture was stirred at room temperature for 0.5 hour. To the above solution was added MeI (402.2 mg; 2.833 mmol; 1.50 eq.). The resulting solution was stirred at room temperature for 2 hours. The desired product was detected via LCMS. The resulting mixture was diluted with EtOAc (50 mL) and washed with brine (3×10 mL). The organic layer was concentrated under vacuum. The residue was purified by flash chromatography on silica gel column using 5-10% of EtOAc in petroleum ether as eluent to provide 5-bromo-2-(methoxymethyl)benzofuran as a white solid (353.3 mg, 76.7%). LCMS (ESI) m/z 241.0, [M+H]⁺.

### Step 3: N-(5-((2-(methoxymethyl)benzofuran-5-yl)ethynyl)-8-(methylamino)-2,7-naphthyridin-3-yl)cyclopropanecarboxamide

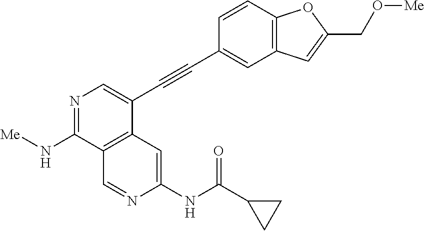 [SMILES: CNc1ncc(C#Cc2ccc3oc(COC)cc3c2)c2cc(NC(=O)C3CC3)ncc12]

N-(5-((2-(methoxymethyl)benzofuran-5-yl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 5-bromo-2-(methoxymethyl)benzofuran as the starting material. LCMS (ESI) m/z 427.2, [M+H]⁺. ¹H NMR (400 MHz, DMSO-d₆) δ 11.08 (s, 1H), 9.38 (s, 1H), 8.69 (s, 1H), 8.32-8.30 (m, 2H), 7.85 (d, J=1.2 Hz, 1H), 7.67 (d, J=8.4 Hz, 1H), 7.51-7.49 (m, 1H) 6.95 (s, 1H) 4.57 (s, 2H), 3.34 (s, 3H), 3.03 (d, J=4.4 Hz, 3H), 2.12-2.07 (m, 1H), 0.92-0.90 (m, 4H).

### Example 718: Synthesis of N-(5-((4-fluoro-5-methoxypyridin-2-yl)ethynyl)-8-(methylamino)-2,7-naphthyridin-3-yl)cyclopropanecarboxamide

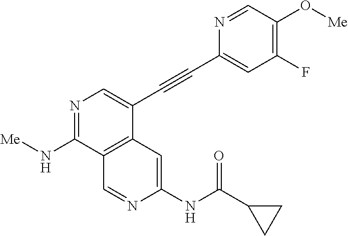 [SMILES: CNc1ncc(C#Cc2cc(F)c(OC)cn2)c2cc(NC(=O)C3CC3)ncc12]

### Step 1: 2-bromo-4-fluoro-5-methoxypyridine

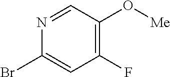

To a stirred solution of 6-bromo-4-fluoropyridin-3-ol (200.0 mg; 1.042 mmol; 1.00 eq.) and Cs₂CO₃ (1.02 g; 3.126 mmol; 3.00 eq.) in DMF (3 mL) was added MeI (147.8 mg; 1.042 mmol; 1.00 eq.) dropwise at 0° C. under nitrogen atmosphere. The resulting mixture was stirred at room temperature for 1 hour. The reaction was quenched by the addition of water (5 mL) at room temperature. The resulting mixture was extracted with EtOAc (3×15 mL). The combined organic layers were washed with brine (2×10 mL), dried over anhydrous Na₂SO₄. After filtration, the filtrate was concentrated under reduced pressure. The residue was purified by flash chromatography on silica gel column using 0-15% of EtOAc in petroleum ether as eluent to provide 2-bromo-4-fluoro-5-methoxypyridine as a yellow solid (120.0 mg, 55.9%).

### Step 2: N-(5-((4-fluoro-5-methoxypyridin-2-yl)ethynyl)-8-(methylamino)-2,7-naphthyridin-3-yl)cyclopropanecarboxamide

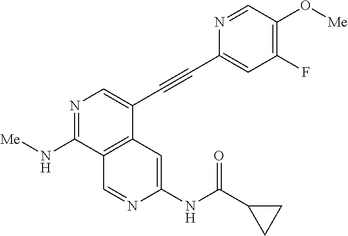 [SMILES: CNc1ncc(C#Cc2cc(F)c(OC)cn2)c2cc(NC(=O)C3CC3)ncc12]

N-(5-((4-fluoro-5-methoxypyridin-2-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 2-bromo-4-fluoro-5-methoxypyridine as the starting material. LCMS (ESI) m/z 392.1, [M+H]⁺. ¹H NMR (400 MHz, DMSO-d₆) δ 11.11 (s, 1H), 9.39 (s, 1H), 8.62 (s, 1H), 8.53 (d, J=10.8 Hz, 1H), 8.45-8.38 (m, 1H), 8.33 (s, 1H), 7.61 (d, J=11.2 Hz, 1H), 4.01 (s, 3H), 3.03 (d, J=4.4 Hz, 3H), 2.13-2.03 (m, 1H), 0.95-0.82 (m, 4H).

### Example 719: Synthesis of N-(5-((2-methoxy-1-methyl-1H-benzo[d]imidazol-6-yl)ethynyl)-8-(methylamino)-2,7-naphthyridin-3-yl)cyclopropanecarboxamide

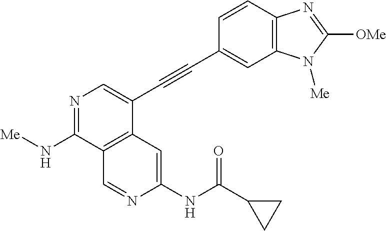 [SMILES: CNc1ncc(C#Cc2ccc3nc(OC)n(C)c3c2)c2cc(NC(=O)C3CC3)ncc12]

### Step 1: 6-bromo-2-methoxy-1-methyl-1H-benzo[d]imidazole

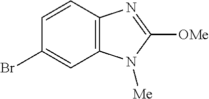 [SMILES: COc1nc2ccc(Br)cc2n1C]

A solution of 6-bromo-2-chloro-1-methyl-1H-benzo[d]imidazole (100.0 mg; 0.407 mmol; 1.00 eq.) and Cs₂CO₃ (265.1 mg; 0.815 mmol; 2.00 eq.) in methanol (1 mL) was stirred at 100° C. for hours under nitrogen atmosphere. The desired product was detected via LCMS. The resulting mixture was concentrated under reduced pressure. The residue was purified by flash chromatography on silica gel column using 10-40% of EtOAc in petroleum ether as eluent to provide 6-bromo-2-methoxy-1-methyl-1H-benzo[d]imidazole as a white solid (66.0 mg, 67%). LCMS (ESI) m/z 241.0, [M+H]⁺.

### Step 2: N-(5-((2-methoxy-1-methyl-1H-benzo[d]imidazol-6-yl)ethynyl)-8-(methylamino)-2,7-naphthyridin-3-yl)cyclopropanecarboxamide

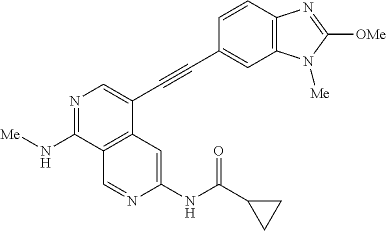 [SMILES: CNc1ncc(C#Cc2ccc3nc(OC)n(C)c3c2)c2cc(NC(=O)C3CC3)ncc12]

N-(5-((2-methoxy-1-methyl-1H-benzo[d]imidazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-2-methoxy-1-methyl-1H-benzo[d]imidazole as the starting material. LCMS (ESI) m/z 427.2, [M+H]⁺. ¹H NMR (400 MHz, DMSO-d₆) δ 11.10 (s, 1H), 9.38 (s, 1H), 8.77 (s, 1H), 8.31-8.25 (m, 2H), 7.69 (d, J=1.2 Hz, 1H), 7.44 (d, J=8.4 Hz, 1H), 7.32-7.27 (m, 1H), 4.1.4 (s, 3H), 3.59 (s, 3H), 3.03 (d, J=4.4 Hz, 3H), 2.15-2.04 (m, 1H), 0.96-0.81 (m, 4H).

### Example 720: Synthesis of N-(5-((3,4-dihydro-1H-benzo[4,5]imidazo[2,1-c][1,4]oxazin-7-yl)ethynyl)-8-(methylamino)-2,7-naphthyridin-3-yl)cyclopropanecarboxamide

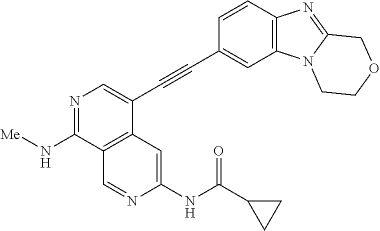 [SMILES: CNc1ncc(C#Cc2ccc3nc4n(c3c2)CCOC4)c2cc(NC(=O)C3CC3)ncc12]

### Step 1: 4-(5-bromo-2-nitrophenyl)morpholine

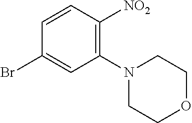 [SMILES: O=[N+]([O-])c1ccc(Br)cc1N1CCOCC1]

A solution of morpholine (0.528 g; 6.060 mmol; 1.00 eq.), 4-bromo-2-fluoro-1-nitrobenzene (2.00 g; 9.091 mmol; 1.50 eq.) and Cs₂CO₃ (3.94 g; 12.121 mmol; 2.00 eq.) in DMF (8 mL) was stirred at 90° C. for 2 hours under nitrogen atmosphere. The desired product was detected via LCMS. The resulting mixture was diluted with EtOAc (80 mL) and washed with brine (3×20 mL). The organic layer was dried over anhydrous Na₂SO₄. After filtration, the filtrate was concentrated under reduced pressure. The residue was purified by flash chromatography on silica gel column using 0-50% of EtOAc in petroleum ether as eluent to provide 4-(5-bromo-2-nitrophenyl)morpholine as a brown solid (1.70 g, 97.7%). LCMS (ESI) m/z 287.0, [M+H]⁺.

### Step 2: 4-bromo-2-morpholinoaniline

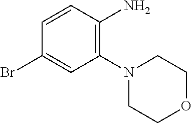 [SMILES: Nc1ccc(Br)cc1N1CCOCC1]

A solution of 4-(5-bromo-2-nitrophenyl)morpholine (2.00 g; 6.966 mmol; 1.00 eq.), Fe (1.94 g; 34.829 mmol; 5.00 eq.) and NH₄Cl (2.98 g; 55.726 mmol; 8.00 eq.) in a mixture solvent of EtOH/H₂O (5:1, 12 mL) was stirred at 80° C. for 2 hours under nitrogen atmosphere. The desired product was detected via LCMS. The resulting mixture was filtered, the filter cake was washed with EtOAc (3×20 mL) and water (3×10 mL). The filtrate was concentrated under reduced pressure and extracted with EtOAc (3×30 mL). The combined organic layers were dried over anhydrous Na₂SO₄. After filtration, the filtrate was concentrated under reduced pressure. The residue was purified by flash chromatography on silica gel column using 0-50% of EtOAc in petroleum ether as eluent to provide 4-bromo-2-morpholinoaniline as a brown solid (1.10 g, 55.8%). LCMS (ESI) m/z 257.0, [M+H]⁺.

### Step 3: 7-bromo-3,4-dihydro-1H-benzo[4,5]imidazo[2,1c][1,4]oxazine

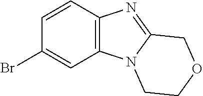 [SMILES: Brc1ccc2nc3n(c2c1)CCOC3]

An oven-dried, nitrogen-flushed 20 mL vessel was charged with a solution of 4-bromo-2-morpholinoaniline (51.4.7 mg; 2.00 mmol; 1.00 eq.) in CH₂Cl₂ (10 mL). To the above solution were added TFA (1.5 mL) and H₂O₂ (30%, 308 uL) dropwise. An exothermic reaction took place and the color darkened. After addition was complete, the solution was stirred under reflux for 3 hours, during which time the color gradually faded. The solution was cooled, the organic solvent was washed with aqueous sodium carbonate and water. The organic layer was dried over anhydrous Na₂SO₄ and evaporated under reduced pressure. The residue was purified by flash chromatography on silica gel column using 0-50% of EtOAc in petroleum ether as eluent to provide 7-bromo-3,4-dihydro-1H-benzo[4,5]imidazo[2,1-c][1,4]oxazine. LCMS (ESI) m/z 252.9, [M+H]⁺.

### Step 4: N-(5-((3,4-dihydro-1H-benzo[4,5]imidazo[2,1-c][1,4]oxazin-7-yl)ethynyl)-8-(methylamino)-2,7-naphthyridin-3-yl)cyclopropanecarboxamide

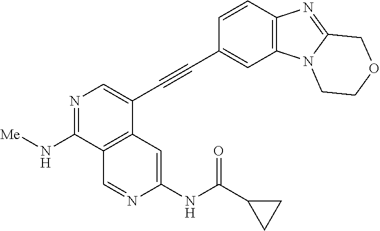 [SMILES: CNc1ncc(C#Cc2ccc3nc4n(c3c2)CCOC4)c2cc(NC(=O)C3CC3)ncc12]

N-(5-((3,4-dihydro-1H-benzo[4,5]imidazo[2,1-c][1,4]oxazin-7-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 7-bromo-3,4-dihydro-1H-benzo[4,5]imidazo[2,1-c][1,4]oxazine as the starting material. LCMS (ESI) m/z 439.2, [M+H]⁺. ¹H NMR (400 MHz, DMSO-d₆) δ 11.10 (s, 1H), 9.38 (s, 1H), 8.75 (s, 1H), 8.40-8.23 (m, 2H), 7.86 (d, J=1.2 Hz, 1H), 7.64 (d, J=8.4 Hz, 1H), 7.41 (dd, J=8.4, 1.2 Hz, 1H), 4.99 (s, 2H), 4.32-4.16 (m, 4H), 3.03 (d, J=4.4 Hz, 3H), 2.16-2.04 (m, 1H), 0.95-0.73 (m, 4H).

### Example 721: Synthesis of 1V-(5-((4-fluoropyrazolo[1,5-a]pyridin-2-yl)ethynyl)-8-(methylamino)-2,7-naphthyridin-3-yl)cyclopropanecarboxamide

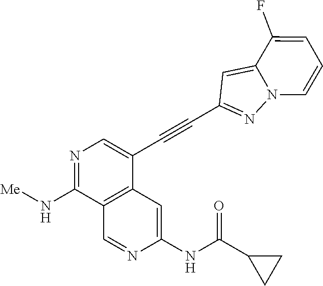 [SMILES: CNc1ncc(C#Cc2cc3c(F)cccn3n2)c2cc(NC(=O)C3CC3)ncc12]

### Step 1: 4-fluoropyrazolo[1,5-a]pyridin-2-ol

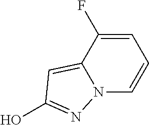 [SMILES: Oc1cc2c(F)cccn2n1]

A solution of methyl 2-(3-fluoropyridin-2-yl)acetate (300.1 mg; 1.772 mmol; 1.00 eq.) and 0-(mesitylsulfonyl)hydroxylamine (1.50 g; 6.962 mmol; 4.00 eq.) in CH₂Cl₂ (3 mL) was stirred at room temperature for 12 hours under nitrogen atmosphere. The desired product was detected via LCMS. The resulting mixture was concentrated under reduced pressure. The residue was purified by flash chromatography on silica gel column using 5-8% of MeOH in CH₂Cl₂ as eluent to provide 4-fluoropyrazolo[1,5-a]pyridin-2-ol as a yellow solid (150.3 mg, 55.6%). LCMS (ESI) m/z 153.0, [M+H]⁺.

### Step 2: 4-fluoropyrazolo[1,5-a]pyridin-2-yl trifluoromethanesulfonate

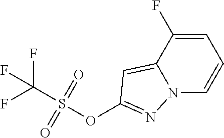 [SMILES: O=S(=O)(Oc1cc2c(F)cccn2n1)C(F)(F)F]

To a stirred solution of 4-fluoropyrazolo[1,5-a]pyridin-2-ol (50.3 mg; 0.312 mmol; 1.00 eq.) and DIEA. (127.3 mg; 0.981 mmol; 3.00 eq.) in CH₂Cl₂ (2 mL) was added dropwise Tf₂O (140.2 mg; 0.491 mmol; 1.50 eq.) at 0° C. under nitrogen atmosphere. The resulting mixture was stirred at room temperature for 2 hours. The desired product was detected via LCMS. The resulting mixture was diluted with CH₂Cl₂ (10 mL) and washed with water (2×10 mL). The organic layer was dried over anhydrous sodium sulfate. After filtration, the filtrate was concentrated under reduced pressure to afford 4-fluoropyrazolo[1,5-a]pyridin-2-yl trifluoromethanesulfonate as a white solid (90.3 mg, crude). LCMS (ESI) m/z 285.0, [M+H]⁺.

### Step 3: N-(5-((4-fluoropyrazolo[1,5-a]pyridin-2-yl)ethynyl)-8-(methylamino)-2,7-naphthyridin-3-yl)cyclopropanecarboxamide

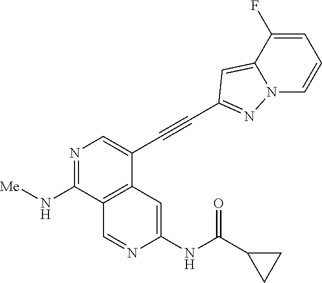 [SMILES: CNc1ncc(C#Cc2cc3c(F)cccn3n2)c2cc(NC(=O)C3CC3)ncc12]

N-(5-((4-fluoropyrazolo[1,5-a]pyridin-2-yl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 4-fluoropyrazolo[1,5-a]pyridin-2-yl trifluoromethanesulfonate as the starting material. LCMS (ESI) m/z 401.1, [M+H]⁺. ¹H NMR (400 MHz, DMSO-d₆) δ 11.10 (s, 1H), 9.40 (s, 1H), 8.66-8.59 (m, 2H), 8.49-8.40 (m, 1H), 8.36 (s, 1H), 7.25-7.21 (m, 1H), 7.06 (s, 1H), 7.04-6.96 (m, 1H), 3.04 (d, J=4.4 Hz, 3H), 2.14-2.05 (m, 1H), 0.92-0.80 (m, 4H).

### Example 722: Synthesis of N-(5-((5-(methoxy-d₃)pyridin-2-yl)ethynyl)-8-(methylamino)-2,7-naphthyridin-3-yl)cyclopropanecarboxamide

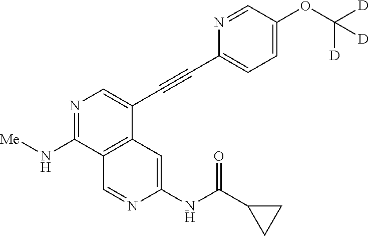 [SMILES: [2H]C([2H])([2H])Oc1ccc(C#Cc2cnc(NC)c3cnc(NC(=O)C4CC4)cc23)nc1]

### Step 1: 2-bromo-5-(methoxy-d₃)pyridine

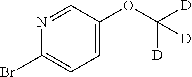 [SMILES: [2H]C([2H])([2H])Oc1ccc(Br)nc1]

To a stirred solution of 6-bromopyridin-3-ol (500.0 mg; 2.874 mmol; 1.00 eq.) in DMF (5 mL) were added Cs₂CO₃ (2.83 g; 8.677 mmol; 3.02 eq.) and CD₃I (410.0 mg; 2.828 mmol; 0.98 eq.) under nitrogen atmosphere. The resulting solution was stirred at room temperature for 12 hours. The resulting mixture was diluted with water (10 mL) and extracted with EtOAc (3×30 mL). The combined organic layers were washed with brine (3×10 mL) and dried over anhydrous Na₂SO₄. After filtration, the filtrate was concentrated under reduced pressure and purified by flash chromatography on silica gel column using 10-15% of EtOAc in petroleum ether as eluent to provide 2-bromo-5-(methoxy-d₃)pyridine as a yellow liquid (340.0 mg, 61.9%).

### Step 2: N-(5-((5-(methoxy-d₃)pyridin-2-yl)ethynyl)-8-(methylamino)-2,7-naphthyridin-3-yl)cyclopropanecarboxamide

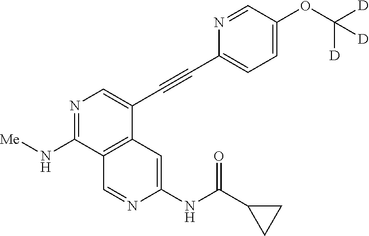 [SMILES: [2H]C([2H])([2H])Oc1ccc(C#Cc2cnc(NC)c3cnc(NC(=O)C4CC4)cc23)nc1]

N-(5-((5-(methoxy-d₃)pyridin-2-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 2-bromo-5-(methoxy-d₃)pyridine as the starting material. LCMS (ESI) m/z 377.2, [M+H]⁺. ¹H NMR (400 MHz, DMSO-d₆) δ 11.07 (s, 1H), 9.38 (s, 1H), 8.61 (s, 1H), 8.37-8.30 (m, 3H), 7.59 (d, J=8.8 Hz, 1H), 7.47 (dd, J=8.8, 3.2 Hz, 1H), 3.02 (d, J=4.4 Hz, 3H), 2.12-2.08 (m, 1H), 0.92-0.81 (m, 4H).

### Example 723: Synthesis of 1V-(5-((1-(difluoromethyl)-1H-indazol-5-yl)ethynyl)-8-(methylamino)-2,7-naphthyridin-3-yl)cyclopropanecarboxamide

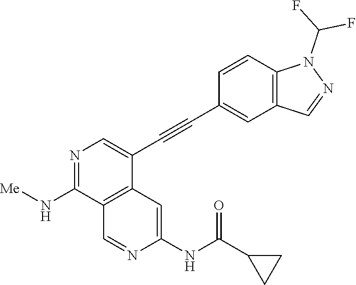 [SMILES: CNc1ncc(C#Cc2ccc3c(cnn3C(F)F)c2)c2cc(NC(=O)C3CC3)ncc12]

### Step 1: 5-bromo-1-(difluoromethyl)-1H-indazole

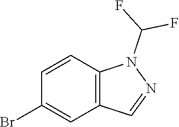 [SMILES: FC(F)n1ncc2cc(Br)ccc21]

To a stirred solution of diethyl (bromodifluoromethyl)phosphonate (339.0 mg; 1.270 mmol; 1.00 eq.) in MeCN (4 mL) were added 5-bromo-1H-indazole (500.3 mg, 2.539 mmol, 2.00 eq.) and KF (147.5 mg, 2.539 mmol, 2.00 eq.) under nitrogen atmosphere. The resulting solution was stirred at room temperature for 12 hours. The resulting mixture was concentrated under reduced pressure. The residue was purified by flash chromatography on silica gel column using 15-20% of EtOAc in petroleum ether as eluent to provide 5-bromo-1-(difluoromethyl)-1H-indazole as a yellow solid (300 mg, 95.6%). LCMS (ESI) m/z 247.0, [M+H]⁺.

### Step 2: N-(5-((1-(difluoromethyl)-1H-indazol-5-yl)ethynyl)-8-(methylamino)-2,7-naphthyridin-3-yl)cyclopropanecarboxamide

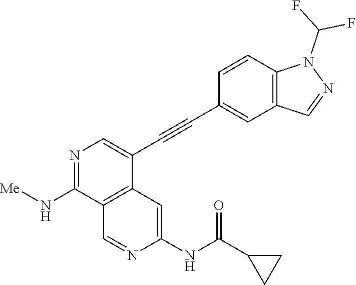 [SMILES: CNc1ncc(C#Cc2ccc3c(cnn3C(F)F)c2)c2cc(NC(=O)C3CC3)ncc12]

N-(5-((1-(difluoromethyl)-1H indazol-5-yl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 5-bromo-1-(difluoromethyl)-1H-indazole as the starting material. LCMS (ESI) m/z 433.2, [M+H]⁺. ¹H NMR (400 MHz, DMSO-d₆) δ 11.10 (s, 1H), 9.39 (s, 1H), 8.96 (s, 1H), 8.69 (s, 1H), 8.35-8.27 (m, 2H), 8.17 (t, J=58.8 Hz, 1H), 8.05 (s, 1H), 7.80 (d, J=8.8 Hz, 1H), 7.51 (d, J=8.8 Hz, 1H), 3.02 (d, J=4.4 Hz, 3H), 2.13-2.06 (m, 1H), 0.92-0.82 (m, 4H).

### Example 724: Synthesis of N-(5-((3-(2-hydroxyethyl)pyridin-4-yl)ethynyl)-8-(methylamino)-2,7-naphthyridin-3-yl)cyclopropanecarboxamide

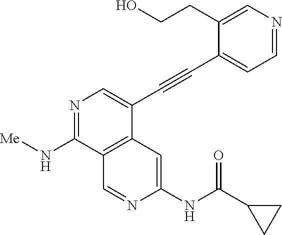 [SMILES: CNc1ncc(C#Cc2ccncc2CCO)c2cc(NC(=O)C3CC3)ncc12]

### Step 1: 4-chloro-3-vinylpyridine

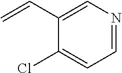

To a stirred solution of methyltriphenylphosphonium bromide (10.09 g; 28.246 mmol; 2.00 eq.) in ether (250 mL) was added n-BuLi (2.5 M in hexane, 11.3 mL) dropwise at −78° C. under nitrogen atmosphere. The resulting solution was stirred at −78° C. for 30 minutes. To the above solution was added a solution of 4-chloronicotinaldehyde (2.00 g; 14.153 mmol; 1.00 eq.) in ether (3 mL) dropwise at −78° C. The resulting solution was stirred at room temperature for 12 hours. The desired product was detected via LCMS. The reaction was quenched with saturated NH₄Cl solution (20 mL) and washed with brine (3×50 mL). The organic layer was dried over anhydrous Na₂SO₄. After filtration, the filtrate was concentrated under reduced pressure. The residue was purified by flash chromatography on silica gel column using 0-10% of EtOAc in petroleum ether as eluent to provide 4-chloro-3-vinylpyridine as a colorless oil (860.0 mg, 40.5%). LCMS (ESI) m/z 140.1, [M+H]⁺.

### Step 2: 2-(4-chloropyridin-3-yl)ethan-1-ol

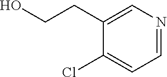

To a stirred solution of 4-chloro-3-vinylpyridine (400.0 mg; 2.866 mmol; 1.00 eq.) in THF (6 mL) was added BH₃THF (1 M in THE, 4 mL) dropwise at 0° C. under nitrogen atmosphere. The resulting solution was stirred at 45° C. for 2 hours. The mixture was allowed to cool down to 0′C. To the above solution were added NaOH (2 M in water, 4.3 mL) and H₂O₂ (30% in water, 0.8 mL) dropwise. The resulting mixture was stirred at room temperature for 2 hours under nitrogen atmosphere. The desired product was detected via LCMS. The reaction was quenched with sat. Na₂S₂O₃ solution and extracted with EtOAc (3×50 mL). The combined organic layers were washed with brine (3×50 mL), dried over anhydrous Na₂SO₄. After filtration, the filtrate was concentrated under reduced pressure. The residue was purified by flash chromatography on silica gel column using 30-70% of EtOAc in petroleum ether as eluent to provide 2-(4-chloropyridin-3-yl)ethan-1-ol as a colorless oil (30.0 mg, 6.3%). LCMS (ESI) m/z 158.0, [M+H]⁺.

### Step 3: N-(5-((3-(2-hydroxyethyl)pyridin-4-yl)ethynyl)-8-(methylamino)-2,7-naphthyridin-3-yl)cyclopropanecarboxamide

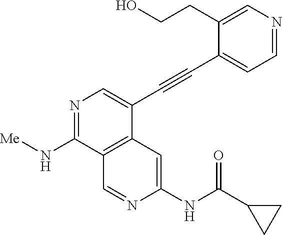 [SMILES: CNc1ncc(C#Cc2ccncc2CCO)c2cc(NC(=O)C3CC3)ncc12]

N-(5-((3-(2-hydroxyethyl)pyridin-4-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 2-(4-chloropyridin-3-yl)ethan-1-ol as the starting material. LCMS (ESI) m/z 388.1, [M+H]⁺. ¹H NMR (400 MHz, DMSO-d₆) δ 11.15 (s, 1H), 9.39 (s, 1H), 8.61 (s, 1H), 8.54 (s, 1H), 8.50-8.40 (m, 2H), 8.37 (s, 1H), 7.43 (d, J=5.2 Hz, 1H), 4.68 (d, J=5.6 Hz, 1H), 3.80-3.72 (m, 2H), 3.10-3.01 (m, 5H), 2.12-2.02 (m, 1H), 0.92-0.85 (m, 4H).

### Example 725: Synthesis of N-(5-((2-(methoxydimethyl)benzo[d]thiazol-6-yl)ethynyl)-8-(methylamino)-2,7-naphthyridin-3-yl)cyclopropanecarboxamide

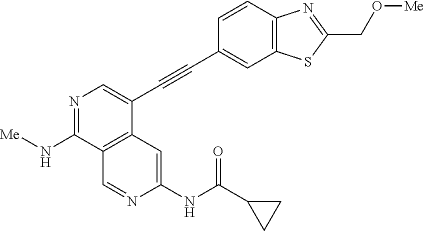 [SMILES: CNc1ncc(C#Cc2ccc3nc(COC)sc3c2)c2cc(NC(=O)C3CC3)ncc12]

### Step 1: (6-bromobenzo[d]thiazol-2-yl)methanol

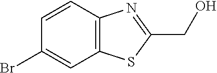 [SMILES: OCc1nc2ccc(Br)cc2s1]

To a solution of methyl 6-bromobenzo[d]thiazole-2-carboxylate (400 mg; 1.470 mmol; 1.00 eq.) in THF (5 mL) was added DIBAL-H (1 M in CH₂Cl₂, 3 mL) dropwise at 0′C. The resulting mixture was stirred at room temperature for 1 hour under nitrogen atmosphere. The desired product was detected via LCMS. The reaction was quenched by the addition of MeOH at 0° C. The resulting mixture was concentrated under reduced pressure. The residue was purified by flash chromatography on pre-packed C18 column using 10-50% of MeCN in water (1.0 mmol/L NH₄HCO₃) to provide (6-bromobenzo[d]thiazol-2-yl)methanol as a white solid (193 mg, 53.7%). LCMS (ESI) m/z 244.1, [M+H]⁺.

### Step 2: 6-bromo-2-(m ethoxymethyl)benzo[d]thiazole

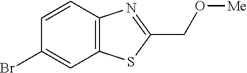 [SMILES: COCc1nc2ccc(Br)cc2s1]

A solution of (6-bromobenzo[d]thiazol-2-yl)methanol (140 mg; 0.435 mmol; 1.00 eq.), Cs₂CO₃ (376 mg; 1.154 mmol; 2.66 eq.) and MeI (82 mg; 0.578 mmol; 1.33 eq.) in DMF (7 mL) was stirred at room temperature for 14 hours under nitrogen atmosphere. The desired product was detected via LCMS. The resulting mixture was diluted with EtOAc (60 mL) and washed with brine (3×10 mL). The organic layer was dried over anhydrous Na₂SO₄. After filtration, the filtrate was concentrated under reduced pressure. The residue was purified by flash chromatography on silica gel column using 30-60% of EtOAc in petroleum ether as eluent to provide 6-bromo-2-(methoxymethyl)benzo[d]thiazole as a yellow solid (127 mg, 85.7%). LCMS (ESI) m/z 258.0, [M+H]⁺.

### Step 3: N-(5-((2-(methoxymethyl)benzo[d]thiazol-6-yl)ethynyl)-8-(methylamino)-2,7-naphthyridin-3-yl)cyclopropanecarboxamide

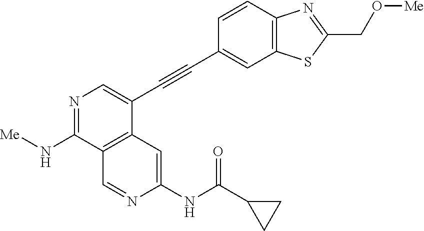 [SMILES: CNc1ncc(C#Cc2ccc3nc(COC)sc3c2)c2cc(NC(=O)C3CC3)ncc12]

N-(5-((2-(methoxymethyl)benzo[d]thiazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-2-(methoxymethyl)benzo[d]thiazole as the starting material. LCMS (ESI) m/z 444.1, [M+H]⁺. ¹H NMR (400 MHz, DMSO-d₆) δ 11.11 (s, 1H), 9.39 (s, 1H), 8.70 (s, 1H), 8.40-8.34 (m, 2H), 8.31 (s, 1H), 8.03 (d, J=8.4 Hz, 1H), 7.71-7.64 (m, 1H), 4.89 (s, 2H), 3.49 (s, 3H), 3.03 (d, J=4.4 Hz, 3H), 2.15-2.04 (m, 1H), 0.94-0.82 (m, 4H).

### Example 726: Synthesis of N-(5-((5-(azetidin-1-yl)pyridin-2-yl)ethynyl)-8-(methylamino)-2,7-naphthyridin-3-yl)cyclopropanecarboxamide

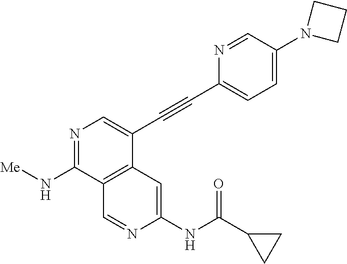 [SMILES: CNc1ncc(C#Cc2ccc(N3CCC3)cn2)c2cc(NC(=O)C3CC3)ncc12]

### Step 1: 5-(azetidin-1-yl)-2-chloropyridine

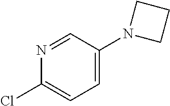 [SMILES: Clc1ccc(N2CCC2)cn1]

A solution of 2-chloro-5-iodopyridine (500.0 mg; 2.088 mmol; 1.00 eq.), azetidine (143.0 mg; 2.505 mmol; 1.20 eq.), Pd₂(dba)₃ (191.2 mg; 0.209 mmol; 0.10 eq.), XantPhos (241.7 mg; 0.418 mmol; 0.20 eq.) and Cs₂CO₃ (1360.8 mg; 4.177 mmol; 2.00 eq.) in 1,4-dioxane (8 mL) was stirred at 70° C. for 14 hours under nitrogen atmosphere. The desired product was detected via LCMS. The solvent was concentrated under vacuum. The residue was purified by flash chromatography on silica gel column using 10-30% of EtOAc in petroleum ether as eluent to provide 5-(azetidin-1-yl)-2-chloropyridine as a yellow solid (130.0 mg, 35.1%). LCMS (ESI) m/z 169.1, [M+H]⁺.

### Step 2: N-(5-((5-(azetidin-1-yl)pyridin-2-yl)ethynyl)-8-(methylamino)-2,7-naphthyridin-3-yl)cyclopropanecarboxamide

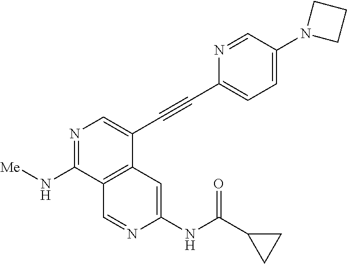 [SMILES: CNc1ncc(C#Cc2ccc(N3CCC3)cn2)c2cc(NC(=O)C3CC3)ncc12]

N-(5-((5-(azetidin-1-yl)pyridin-2-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 5-(azetidin-1-yl)-2-chloropyridine and 5-(azetidin-1-yl)-2-chloropyridine as the starting material. LCMS (ESI) m/z 399.2, [M+H]⁺. ¹H NMR (400 MHz, DMSO-d₆) δ 11.07 (s, 1H), 9.37 (s, 1H), 8.61 (s, 1H), 8.37-8.30 (m, 1H), 8.27 (s, 1H), 7.80 (d, =2.8 Hz, 1H), 7.41 (d, J=8.8 Hz, 1H), 6.95-6.74 (m, 1H), 4.08-3.88 (m, 4H), 3.02 (d, J=4.4 Hz, 3H), 2.42-2.33 (m, 2H), 2.11-2.02 (m, 1H), 0.91-0.80 (m, 4H).

### Example 727: Synthesis of N-(5-((5-(2-(methoxymethyl)azetidin-1-yl)pyridin-2-yl)ethynyl)-8-(methylamino)-2,7-naphthyridin-3-yl)cyclopropanecarboxamide

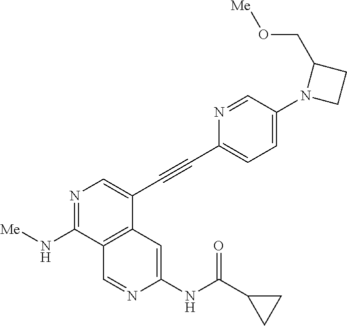 [SMILES: CNc1ncc(C#Cc2ccc(N3CCC3COC)cn2)c2cc(NC(=O)C3CC3)ncc12]

### Step 1: 2-bromo-5-(2-(methoxymethyl)azetidin-1-yl)pyridine

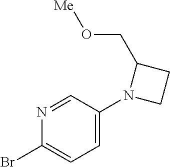 [SMILES: COCC1CCN1c1ccc(Br)nc1]

A mixture of 2-(methoxymethyl)azetidine (106.9 mg; 1.057 mmol; 1.00 eq.), 2-bromo-5-iodopyridine (300.0 mg; 1.057 mmol; 1.00 eq.), Pd₂(dba)₃ (96.8 mg; 0.106 mmol; 0.1.0 eq.), XantPhos (122.3 mg; 0.211 mmol; 0.20 eq.) and Cs₂CO₃ (688.6 mg; 2.113 mmol; 2.00 eq.) in 1,4-dioxane (5 mL) was stirred at 70° C. for 14 hours under nitrogen atmosphere. The desired product was detected via LCMS. The solvent was concentrated under vacuum. The residue was purified by flash chromatography on silica gel column using 10-30% of EtOAc in petroleum ether as eluent to provide 2-bromo-5-[2-(methoxymethyl)azetidin-1-yl]pyridine as a white solid (130.0 mg, 45.5%). LCMS (ESI) m/z 257.1, [M+H]⁺.

### Step 2: N-(5-((5-(2-(methoxymethyl)azetidin-1-yl)pyridin-2-yl)ethynyl)-8-(methylamino)-2,7-naphthyridin-3-yl)cyclopropanecarboxamide

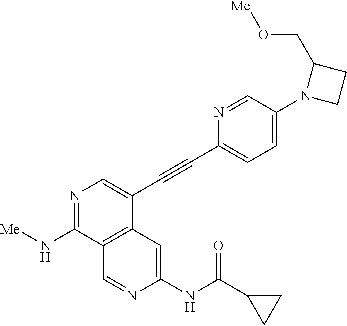 [SMILES: CNc1ncc(C#Cc2ccc(N3CCC3COC)cn2)c2cc(NC(=O)C3CC3)ncc12]

N-(5-((5-(2-(methoxymethyl)azetidin-1-yl)pyridin-2-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 2-bromo-5-(2-(methoxymethyl)azetidin-1-yl)pyridine as the starting material. LCMS (ESI) m/z 443.2, [M+H]⁺. ¹H NMR (400 MHz, DMSO-d₆) δ 11.07 (s, 1H), 9.37 (s, 1H), 8.60 (s, 1H), 8.38-8.30 (m, 1H), 8.27 (s, 1H), 7.96 (d, J=2.4 Hz, 1H), 7.40 (d, J=8.4 Hz, 1H), 7.01-6.98 (m, 1H), 4.35-4.24 (m, 1H), 4.02-3.92 (m, 1H), 3.74-3.66 (m, 1H), 3.65-3.56 (m, 2H), 3.35 (s, 3H), 3.02 (d, J=4.4 Hz, 3H), 2.40-2.35 (m, 1H), 2.22-2.15 (m, 1H), 2.12-2.02 (m, 1H), 0.92-0.80 (m, 4H).

### Example 728: Synthesis of N-(8-(methylamino)-5-((5-(morpholinomethyl)pyridin-2-yl)ethynyl)-2,7-naphthyridin-3-yl)cyclopropanecarboxamide

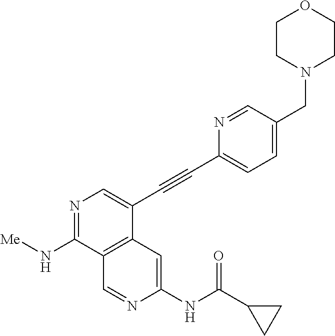 [SMILES: CNc1ncc(C#Cc2ccc(CN3CCOCC3)cn2)c2cc(NC(=O)C3CC3)ncc12]

### Step 1: 4-((6-bromopyridin-3-yl)methyl)morpholine

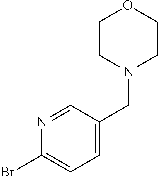 [SMILES: Brc1ccc(CN2CCOCC2)cn1]

To a stirred solution of 6-bromonicotinaldehyde (200.0 mg; 1.075 mmol; 1.00 eq.) and morpholine (141.4 mg; 1.612 mmol; 1.51 eq.) in CH₂Cl₂ (20 mL) were added NaBH(OAc)₃ (683.6 mg; 3.225 mmol; 3.00 eq.) and AcOH (65.0 mg; 1.075 mmol; 1.00 eq.) at 0° C. The resulting solution was stirred at room temperature for 5 hours. The desired product was detected via LCMS. The mixture was basified to pH=8 with NaOH (1 M in water) and extracted with EtOAc (3×50 mL). The combined organic layers were washed with brine (3×50 mL) and dried over anhydrous Na₂SO₄. After filtration, the filtrate was concentrated under reduced pressure. The residue was purified by flash chromatography on silica gel column using 50-80% of EtOAc in petroleum ether as eluent to provide 4-((6-bromopyridin-3-yl)methyl)morpholine as a white solid (200.0 mg, 68.7%). LCMS (ESI) m/z 257.1, [M+H]⁺.

### Step 2: N-(8-(methylamino)-5-((5-(morpholinomethyl)pyridin-2-yl)ethynyl)-2,7-naphthyridin-3-yl)cyclopropanecarboxamide

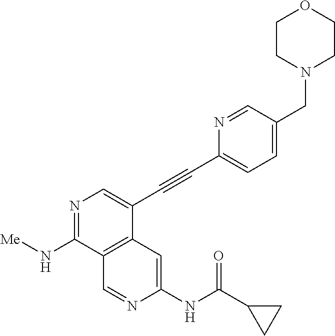 [SMILES: CNc1ncc(C#Cc2ccc(CN3CCOCC3)cn2)c2cc(NC(=O)C3CC3)ncc12]

N-(8-(methylamino)-5-((5-(morpholinomethyl)pyridin-2-yl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 4-((6-bromopyridin-3-yl)methyl)morpholine as the starting material. LCMS (ESI) m/z 443.2, [M+H]⁺. ¹H NMR (400 MHz, DMSO-d₆) δ 11.10 (s, 1H), 9.38 (s, 1H), 8.63 (s, 1H), 8.53 (d, J=1.6 Hz, 110, 8.44-8.39 (m, 1H), 8.35 (s, 1H), 7.82-7.77 (m, 1H), 7.60 (d, J=8.0 Hz, 1H), 3.61-3.57 (m, 4H), 3.54 (s, 2H), 3.03 (d, J=4.4 Hz, 3H), 2.42-2.36 (m, 4H), 2.15-2.06 (m, 1H), 0.92-0.82 (m, 4H).

### Example 729: Synthesis of N-(5-((5-(1,1-difluoroethyl)pyridin-2-yl)ethynyl)-8-(methylamino)-2,7-naphthyridin-3-yl)cyclopropanecarboxamide

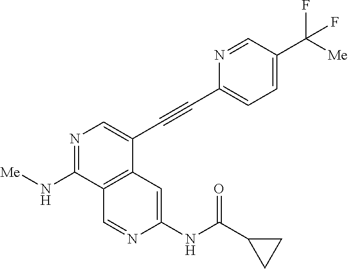 [SMILES: CNc1ncc(C#Cc2ccc(C(C)(F)F)cn2)c2cc(NC(=O)C3CC3)ncc12]

### Step 1: 2-bromo-5-(1,1-difluoroethyl)pyridine

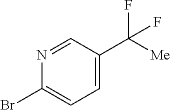 [SMILES: CC(F)(F)c1ccc(Br)nc1]

A solution of 1-(6-bromopyridin-3-yl)ethanone (500.0 mg; 2.500 mmol; 1.00 eq.) in DAST (5 mL) was stirred at room temperature for 14 hours. The desired product was detected via LCMS. The solution was added into a saturated sodium bicarbonate solution (100 mL), extracted with CH₂Cl₂ (3×50 mL). The combined organic layers were washed with brine (1×50 mL) and dried over anhydrous Na₂SO₄. After filtration, the filtrate was concentrated under reduced pressure. The residue was purified by flash chromatography on silica gel column using 5-11% of EtOAc in petroleum ether as eluent to provide 2-bromo-5-(1,1-difluoroethyl)pyridine as a yellow oil (399.4 mg, 71.9%). LCMS (ESI) m/z 222.0, [M+H]⁺.

### Step 2: N-(5-((5-(1,1-difluoroethyl)pyridin-2-yl)ethynyl)-8-(methylamino)-2,7-naphthyridin-3-yl)cyclopropanecarboxamide

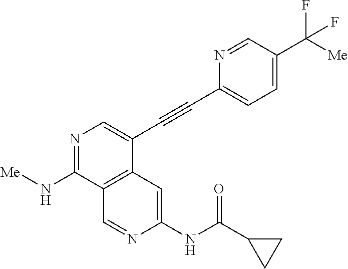 [SMILES: CNc1ncc(C#Cc2ccc(C(C)(F)F)cn2)c2cc(NC(=O)C3CC3)ncc12]

N-(5-((5-(1,1-difluoroethyl)pyridin-2-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-1.5 yl)cyclopropanecarboxamide and 2-bromo-5-(1,1-difluoroethyl)pyridine as the starting material. LCMS (ESI) m/z 408.2, [M+H]⁺. ¹H NMR (400 MHz, DMSO-d₆) δ 11.11 (s, 1H), 9.39 (s, 1H), 8.82 (d, J=2.0 Hz, 1H), 8.64 (s, 1H), 8.51-8.47 (m, 1H), 8.39 (s, 1H), 8.08 (dd, J=8.4, 2.0 Hz, 1H), 7.73 (d, J=8.4 Hz, 1H), 3.04 (d, J=4.4 Hz, 3H), 2.11-2.01 (m, 3H), 2.12-2.03 (m, 1H), 0.91-0.84 (m, 4H).

### Example 730: Synthesis of N-(5-((4-cyano-5-methoxypyridin-2-yl)ethynyl)-8-(methylamino)-2,7-naphthyridin-3-yl)cyclopropanecarboxamide

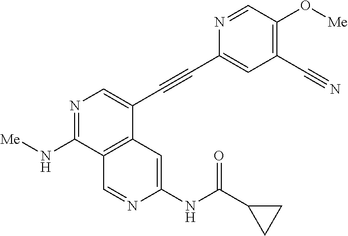 [SMILES: CNc1ncc(C#Cc2cc(C#N)c(OC)cn2)c2cc(NC(=O)C3CC3)ncc12]

### Step 1: 2-bromo-5-methoxyisonicotinonitrile

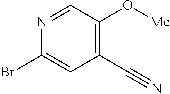 [SMILES: COc1cnc(Br)cc1C#N]

To a stirred solution of 2-bromo-5-hydroxyisonicotinonitrile (100.0 mg; 0.502 mmol; 1.00 eq.) and Cs₂CO₃ (491.0 mg; 1.506 mmol; 3.00 eq.) in DMF (2 mL) was added MeI (71.3 mg; 0.502 mmol; 1.00 eq.) dropwise at 0° C. under nitrogen atmosphere. The resulting mixture was stirred at room temperature for 30 minutes. The desired product was detected via LCMS. The mixture was diluted with EtOAc (30 mL) and washed with brine (2×1.0 mL). The organic layer was dried over anhydrous Na₂SO₄. After filtration, the filtrate was concentrated under reduced pressure. The residue was purified by flash chromatography on silica gel column using 10-40% of EtOAc in petroleum ether as eluent to provide 2-bromo-5-methoxyisonicotinonitrile as a white solid (70.1 mg, 65.2%). LCMS (ESI) m/z 213.0, [M+H]⁺.

### Step 2: 1-(5-((4-cyano-5-methoxypyridin-2-yl)ethynyl)-8-(methylamino)-2,7-naphthyridin-3-yl)cyclopropanecarboxamide

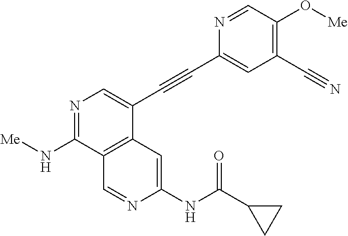 [SMILES: CNc1ncc(C#Cc2cc(C#N)c(OC)cn2)c2cc(NC(=O)C3CC3)ncc12]

N-(5-((4-cyano-5-methoxypyridin-2-yl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 2-bromo-5-methoxyisonicotinonitrile as the starting material. LCMS (ESI) m/z 399.1, [M+H]⁺.

### Example 731: Synthesis of N-(5-((4-(2-fluoro-2-methylpropoxy)phenyl)ethynyl)-8-(methylamino)-2,7-naphthyridin-3-yl)cyclopropanecarboxamide

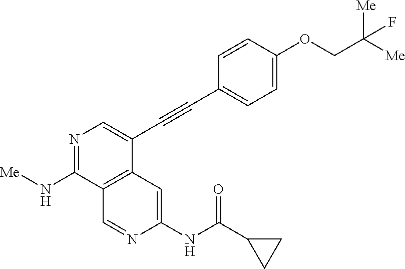 [SMILES: CNc1ncc(C#Cc2ccc(OCC(C)(C)F)cc2)c2cc(NC(=O)C3CC3)ncc12]

### Step 1: 1-(4-bromophenoxy)-2-methylpropan-2-ol

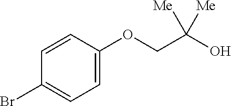 [SMILES: CC(C)(O)COc1ccc(Br)cc1]

A solution of 4-bromophenol (500.0 mg; 2.890 mmol; 1.00 eq.), 1-bromo-2-methylpropan-2-ol (486.2 mg; 3.179 mmol; 1.10 eq.) and Cs₂CO₃ (1.88 g; 5.780 mmol; 2.00 eq.) in DMF (20 mL) was stirred at 100° C. for 2 hours under nitrogen atmosphere. The resulting mixture was diluted with water (10 mL) and extracted with EtOAc (3×30 mL). The combined organic layers were washed with brine (3×15 mL), dried over anhydrous Na₂SO₄. After filtration, the filtrate was concentrated under reduced pressure. The residue was purified by flash chromatography on silica gel column using 0-15% of EtOAc in petroleum ether as eluent to provide 1-(4-bromophenoxy)-2-methylpropan-2-ol as a white oil (500.1 mg, 70.0%).

### Step 2: 1-bromo-4-(2-fluoro-2-methylpropoxy)benzene

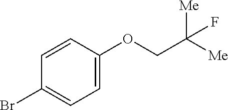 [SMILES: CC(C)(F)COc1ccc(Br)cc1]

To a stirred solution of 1-(4-bromophenoxy)-2-methylpropan-2-ol (50.0 mg; 0.204 mmol; 1.00 eq.) in CH₂Cl₂ (3 mL) was added DAST (65.7 mg; 0.408 mmol; 2.00 eq.) dropwise at 0° C. under nitrogen atmosphere. The resulting mixture was stirred at 0° C. for 1 hour. The reaction was quenched by the addition of saturated sodium bicarbonate solution at 0° C. The resulting mixture was diluted with CH₂Cl₂ (30 mL) and washed with brine (2×10 mL). The organic layer was dried over anhydrous Na₂SO₄. After filtration, the filtrate was concentrated under reduced pressure to afford 1-bromo-4-(2-fluoro-2-methylpropoxy)benzene as an off-white oil. (60.0 mg, crude).

### Step 3: N-(5-((4-(2-fluoro-2-methylpropoxy)phenyl)ethynyl)-8-(methylamino)-2,7-naphthyridin-3-yl)cyclopropanecarboxamide

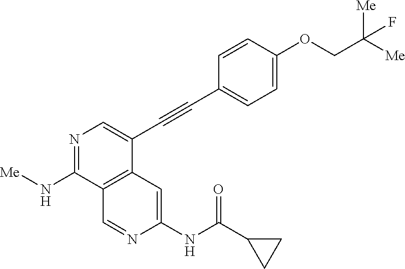 [SMILES: CNc1ncc(C#Cc2ccc(OCC(C)(C)F)cc2)c2cc(NC(=O)C3CC3)ncc12]

N-(5-((4-(2-fluoro-2-methylpropoxy)phenyl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-1.5 yl)cyclopropanecarboxamide and 1-bromo-4-(2-fluoro-2-methylpropoxy)benzene as the starting material. LCMS (ESI) m/z 433.2, [M+H]⁺. ¹H NMR (400 MHz, DMSO-d₆) δ 11.07 (s, 1H), 9.37 (s, 1H), 8.65 (s, 1H), 8.32-8.23 (m, 2H), 7.55-7.47 (m, 2H), 7.11-7.02 (m, 2H), 4.08 (d, J=20.0 Hz, 2H), 3.02 (d, J=4.4 Hz, 3H), 2.14-2.03 (m, 1H), 1.45 (d, J=20.0 Hz, 6H), 0.94-0.80 (m, 4H).

### Example 732: Synthesis of N-(5-((5-(fluoromethoxy)pyridin-2-yl)ethynyl)-8-(methylamino)-2,7-naphthyridin-3-yl)cyclopropanecarboxamide

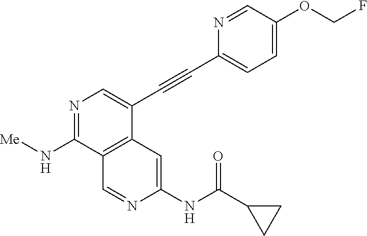 [SMILES: CNc1ncc(C#Cc2ccc(OCF)cn2)c2cc(NC(=O)C3CC3)ncc12]

### Step 1: ethyl 24(6-bromopyridin-3-yl)oxy)acetate

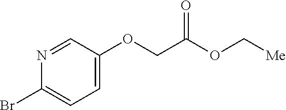 [SMILES: CCOC(=O)COc1ccc(Br)nc1]

To a solution of 6-bromopyridin-3-ol (2.29 g; 13.1.00 mmol; 1.10 eq.) and Cs₂CO₃ (11.7 g; 35.900 mmol; 3.00 eq.) in MeCN (25 mL) was added ethyl 2-bromoacetate (2.00 g; 11.900 mmol; 1.00 eq.) dropwise at 0° C. The resulting mixture was stirred at room temperature for 2 hours. The desired product was detected via LCMS. The resulting mixture was diluted with cold water (30 mL) and extracted with EtOAc (3×30 ml). The combined organic layers were washed with brine (2×1.0 ml) and dried over anhydrous Na₂SO₄. After filtration, the filtrate was concentrated under reduced pressure. The residue was purified by flash chromatography on silica gel column using 10-20% of EtOAc in petroleum ether as eluent to provide ethyl 2-((6-bromopyridin-3-yl)oxy)acetate as a white solid (2.96 g, 91.9%). LCMS (ESI) m/z 260.0, [M+H]⁺.

### Step 2: 2-((6-bromopyridin-3-yl)oxy)acetic acid

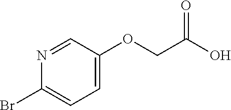 [SMILES: O=C(O)COc1ccc(Br)nc1]

To a stirred solution of ethyl 2-((6-bromopyridin-3-yl)oxy)acetate (2.00 g; 7.690 mmol; 1.00 eq.) in 1,4-dioxane (80 mL) was added a solution of NaOH (282 mg, 7.075 mmol; 0.90 eq.) in water (20 mL). The reaction was stirred at room temperature for 1 hour. The desired product was detected via LCMS. The mixture was acidified to pH=3 with HCl (2 M) and extracted with EtOAc (3×60 mL). The combined organic layers were washed with brine (3×15 mL), dried over anhydrous Na₂SO₄. After filtration, the filtrate was concentrated under reduced pressure to provide 2-((6-bromopyridin-3-yl)oxy)acetic acid as a white solid (1.68 g, 82.9%). LCMS (ESI) m/z 232.1, [M+H]⁺.

### Step 3: 2-bromo-5-(fluoromethoxy)pyridine

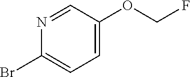

To a solution of 2-((6-bromopyridin-3-yl)oxy)acetic acid (400.0 mg; 1.720 mmol; 1.00 eq.), 1-(chloromethyl)-4-fluoro-1,4-diazabicyclo[2.2.2]octane-1,4-diium tetrafluoroborate (2.14 g; 6.034 mmol; 3.50 eq.) and Ru(bpy)₃Cl₂ (12.9 mg; 0.017 mmol; 0.01 eq.) in a mixture solvent of water/MeCN (1:1, 9.2 mL) was added NaOH (103.4 mg; 2.586 mmol; 1.50 eq.) under nitrogen atmosphere. The reaction mixture was irradiated under a 450 W visible light lamp for 12 hours. The solvent was concentrated under vacuum. The residue was purified by flash chromatography on pre-packed C18 column using 20-100% of MeCN in water (10 mmol/L NH₄HCO₃) as eluent to provide 2-bromo-5-(fluoromethoxy)pyridine as a yellow oil (34.0 mg, 9.6%). LCMS (ESI) m/z 206.0.

### Step 4: N-(5-((5-(fluoromethoxy)pyridin-2-yl)ethynyl)-8-(methylamino)-2,7-naphthyridin-3-yl)cyclopropanecarboxamide

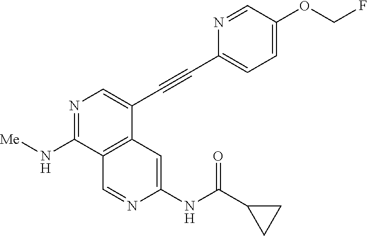 [SMILES: CNc1ncc(C#Cc2ccc(OCF)cn2)c2cc(NC(=O)C3CC3)ncc12]

N-(5-((5-(fluoromethoxy)pyridin-2-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 2-bromo-5-(fluoromethoxy)pyridine as the starting material. LCMS (ESI) m/z 392.1, [M+H]⁺. ¹H NMR (400 MHz, DMSO-d₆) δ 11.08 (s, 1H), 9.39 (s, 1H), 8.62 (s, 1H), 8.50-8.39 (m, 2H), 8.34 (s, 1H), 7.70-7.61 (m, 2H), 5.98 (d, J=53.2 Hz, 2H), 3.03 (d, J=4.4 Hz, 3H), 2.15-2.02 (m, 1H), 0.97-0.76 (m, 4H).

### Example 733: Synthesis of N-(5-((5-(7-methyl-9-oxa-3,7-diazabicyclo[3.3.1]nonan-3-yl)pyridin-2-yl)ethynyl)-8-(methylamino)-2,7-naphthyridin-3-yl)cyclopropanecarboxamide

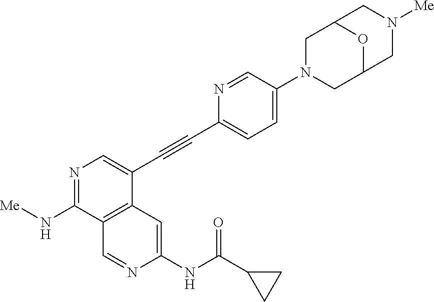 [SMILES: CNc1ncc(C#Cc2ccc(N3CC4CN(C)CC(C3)O4)cn2)c2cc(NC(=O)C3CC3)ncc12]

### Step 1: tert-butyl 7-(6-bromopyridin-3-yl)-9-oxa-3,7-diazabicyclo[3.3.1]nonane-3-carboxylate

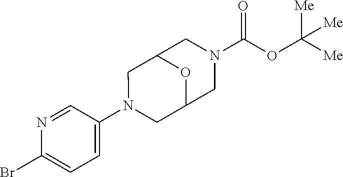 [SMILES: CC(C)(C)OC(=O)N1CC2CN(c3ccc(Br)nc3)CC(C1)O2]

A solution of 2-bromo-5-iodopyridine (200.0 mg; 0.704 mmol; 1.00 eq.), tert-butyl 9-oxa-3,7-diazabicyclo[3.3.1]nonane-3-carboxylate (192.9 mg; 0.845 mmol; 1.20 eq.), Pd₂(dba)₃ (64.5 mg; 0.070 mmol; 0.10 eq.), XantPhos (81.5 mg; 0.141 mmol; 0.20 eq.) and Cs₂CO₃ (459.1 mg; 1.409 mmol; 2.00 eq.) in 1,4-dioxane (10 mL) was stirred at 90° C. for 14 hours under nitrogen atmosphere. The desired product was detected via LCMS. The resulting mixture was concentrated under reduced pressure. The residue was purified by flash chromatography on silica gel column using 30-70% of EtOAc in petroleum ether as eluent to provide tert-butyl 7-(6-bromopyridin-3-yl)-9-oxa-3,7-diazabicyclo[3.3.1]nonane-3-carboxylate as a yellow solid (97.3 mg, 35.9%). LCMS (ESI) m/z 384.1, [M+H]⁺.

### Step 2: 3-(6-bromopyridin-3-yl)-9-oxa-3,7-diazabicyclo[3.3.1]nonane hydrochloride

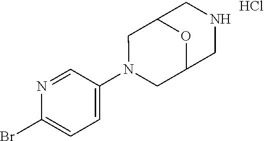 [SMILES: Brc1ccc(N2CC3CNCC(C2)O3)cn1 ; Cl]

To a solution of tert-butyl 7-(6-bromopyridin-3-yl)-9-oxa-3,7-diazabicyclo[3.3.1]nonane-3-carboxylate (130.0 mg; 0.338 mmol; 1.00 eq.) in MeOH (10 mL) was added HCl (gas) (4 M in 1,4-dioxane, 3 mL). The resulting solution was stirred at room temperature for 1.5 hours. The desired product was detected via LCMS. The resulting mixture was concentrated under reduced pressure to afford 3-(6-bromopyridin-3-yl)-9-oxa-3,7-diazabicyclo[3.3.1]nonane hydrochloride as a yellow solid (130 mg, crude). LCMS (ESI) m/z 284.0, [M+H]⁺.

### Step 3: 3-(6-bromopyridin-3-yl)-7-methyl-9-oxa-3,7-diazabicyclo[3.3.1]nonane

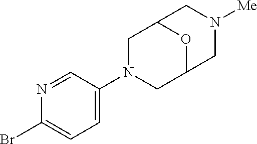 [SMILES: CN1CC2CN(c3ccc(Br)nc3)CC(C1)O2]

To a solution of 3-(6-bromopyridin-3-yl)-9-oxa-3,7-diazabicyclo[3.3.1]nonane hydrochloride (130.0 mg; 0.457 mmol; 1.00 eq.), HCHO (37%) (741 mg; 9.140 mmol; 20 eq.) and DIPEA (176.8 mg; 1.368 mmol; 2.99 eq.) in a mixture solvent of dichloroethane/MeOH (3:1, 16 mL) was added NaBH(OAc)₃ (290.1 mg; 1.369 mmol; 2.99 eq.). The resulting solution was stirred at room temperature for 1.5 hours under nitrogen atmosphere. The desired product was detected via LCMS. The resulting mixture was concentrated under reduced pressure. The residue was purified by flash chromatography on pre-packed C18 column using 25-35% of MeOH in water (10 mmol/L NH₄HCO₃) as eluent to provide 3-(6-bromopyridin-3-yl)-7-methyl-9-oxa-3,7-diazabicyclo[3.3.1]nonane as a white solid (80.1 mg, 58.7%). LCMS (ESI) m/z 298.0, [M+H]⁺.

### Step 4: N-(5-((5-(7-methyl-9-oxa-3,7-diazabicyclo[3.3.1]nonan-3-yl)pyridin-2-yl)ethynyl)-8-(methylamino)-2,7-naphthyridin-3-yl)cyclopropanecarboxamide

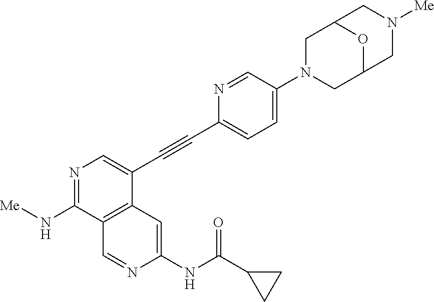 [SMILES: CNc1ncc(C#Cc2ccc(N3CC4CN(C)CC(C3)O4)cn2)c2cc(NC(=O)C3CC3)ncc12]

N-(5-((5-(7-methyl-9-oxa-3,7-diazabicyclo[3.3.1]nonan-3-yl)pyridin-2-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 3-(6-bromopyridin-3-yl)-7-methyl-9-oxa-3,7-diazabicyclo[3.3.1]nonane as the starting material. LCMS (ESI) m/z 484.2, [M+H]⁺. ^{a}H NMR (400 MHz, DMSO-d₆) δ 11.06 (s, 1H), 9.37 (s, 1H), 8.62 (s, 1H), 8.31-8.28 (m, 2H), 8.18 (d, J=2.8 Hz, 1H), 7.44 (d, J=8.4 Hz, 1H), 7.18 (dd, J=8.4, 2.8 Hz, 1H), 4.10-4.02 (m, 2H), 3.73 (d, J=° 12.0 Hz, 2H), 3.21-3.14 (m, 2H), 3.03 (d, J=4.4 Hz, 3H), 2.85 (d, J=12.0 Hz, 2H), 2.34-2.26 (m, 2H), 2.12-2.05 (m, 4H), 0.91-0.88 (m, 4H).

### Example 734: Synthesis of N-(5-((4-(7-methyl-9-oxa-3,7-diazabicyclo[3.3.1]nonan-3-yl)phenyl)ethynyl)-8-(methylamino)-2,7-naphthyridin-3-yl)cyclopropanecarboxamide

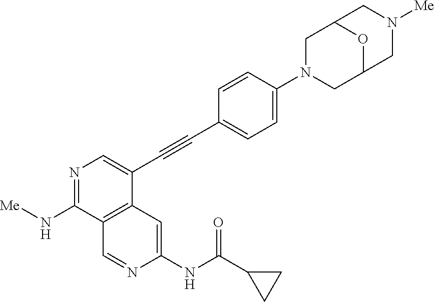 [SMILES: CNc1ncc(C#Cc2ccc(N3CC4CN(C)CC(C3)O4)cc2)c2cc(NC(=O)C3CC3)ncc12]

### Step 1: tert-butyl 7-(4-bromophenyl)-9-oxa-3,7-diazabicyclo[3.3.1]nonane-3-carboxylate

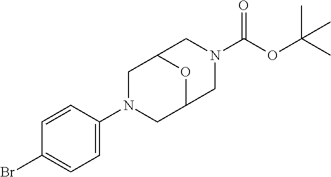 [SMILES: CC(C)(C)OC(=O)N1CC2CN(c3ccc(Br)cc3)CC(C1)O2]

To a stirred solution of 1,4-dibromobenzene (200.0 mg; 0.848 mmol; 1.00 eq.) and tert-butyl 9-oxa-3,7-diazabicyclo[3.3.1]nonane-3-carboxylate (200.0 mg; 0.876 mmol; 1.03 eq.) in toluene (2 mL) were added Pd₂(dba)₃ (80.0 mg; 0.087 mmol; 0.10 eq.), BINAP (110.0 mg; 0.177 mmol; 0.21 eq.) and sodium 2-methylpropan-2-olate (117.0 mg; 1.217 mmol; 1.44 eq.) under nitrogen atmosphere. The resulting mixture was stirred at 80° C. for 12 hours. The desired product was detected via LCMS. The mixture was allowed to cool down to room temperature. The resulting mixture was concentrated under reduced pressure. The residue was purified by flash chromatography on silica gel column using 10-30% of EtOAc in petroleum ether as eluent to provide tert-butyl 7-(4-bromophenyl)-9-oxa-3,7-diazabicyclo[3.3.1]nonane-3-carboxylate as a yellow solid (100.0 mg, 31.1%). LCMS (ESI) m/z 383.1, [M+H]⁺.

### Step 2: 3-(4-bromophenyl)-9-oxa-3,7-diazabicyclo[3.3.1]nonane

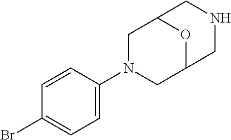 [SMILES: Brc1ccc(N2CC3CNCC(C2)O3)cc1]

To a stirred solution of tert-butyl 7-(4-bromophenyl)-9-oxa-3,7-diazabicyclo[3.3.1]nonane-3-carboxylate (120.0 mg; 0.313 mmol; 1.00 eq.) in MeOH (3 mL) was added HCl (gas) (4 M in 1,4-dioxane, 2 mL) at room temperature. The resulting solution was stirred at room temperature for 2 h. The desired product was detected via LCMS. The resulting mixture was concentrated under reduced pressure. The residue was purified by flash chromatography on pre-packed C18 column using 20-60% of MeCN in water (1.0 mmol/L NH HCO₃) to provide 3-(4-bromophenyl)-9-oxa-3,7-diazabicyclo[3.3.1]nonane as a white solid (50.0 mg, 56.0%). LCMS (ESI) m/z 283.0, [M+H]⁺.

### Step 3: 3-(4-bromophenyl)-7-methyl-9-oxa-3,7-diazabicyclo[3.3.1]nonane

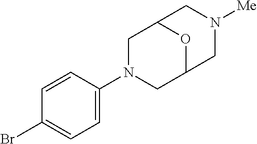 [SMILES: CN1CC2CN(c3ccc(Br)cc3)CC(C1)O2]

To a stirred solution of 3-(4-bromophenyl)-9-oxa-3,7-diazabicyclo[3.3.1]nonane (100.0 mg; 0.353 mmol; 1 eq.) in CH₂Cl₂ (3 mL) were added HCHO (35% in water, 1 mL) and DIEA (136.9 mg; 1.059 mmol; 3 eq.). The resulting mixture was stirred at room temperature for 10 minutes. To the above solution was added NaBH(OAc)₃ (224.5 mg; 1.059 mmol; 3.00 eq.). The resulting mixture was stirred at room temperature for 2 hours under nitrogen atmosphere. The desired product was detected via LCMS. The mixture was concentrated under reduced pressure. The residue was purified by flash chromatography on pre-packed C18 column using 20-60% of MeOH in water (10 mmol/L NH₄HCO₃) to provide 3-(4-bromophenyl)-7-methyl-9-oxa-3,7-diazabicyclo[3.3.1]nonane as a light yellow solid (80.0 mg, 76.1%). LCMS (ESI) m/z 297.1, [M+H]⁺.

### Step 4: N-(5-((4-(7-methyl-9-oxa-3,7-diazabicyclo[3.3.1]nonan-3-yl)phenyl)ethynyl)-8-(methylamino)-2,7-naphthyridin-3-yl)cyclopropanecarboxamide

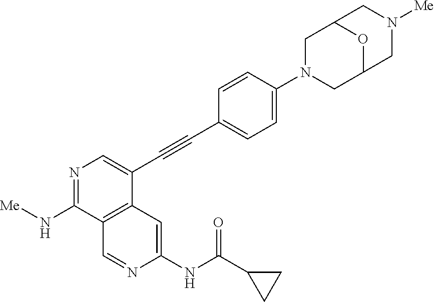 [SMILES: CNc1ncc(C#Cc2ccc(N3CC4CN(C)CC(C3)O4)cc2)c2cc(NC(=O)C3CC3)ncc12]

N-(5-04-(7-methyl-9-oxa-3,7-diazabicyclo[3.3.1]nonan-3-yl)phenyl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 3-(4-bromophenyl)-7-methyl-9-oxa-3,7-diazabicyclo[3.3.1]nonane as the starting material. LCMS (ESI) m/z 483.2, [M+H]⁺. ¹H NMR (400 MHz, DMSO-d₆) δ 11.05 (s, 1H), 9.36 (s, 1H), 8.65 (s, 1H), 8.24-8.18 (m, 2H), 7.42-7.38 (m, 2H), 6.85-6.81 (m, 2H), 4.11-4.05 (m, 2H), 3.72-3.65 (m, 2H), 3.12-3.08 (m, 2H), 3.01 (d, J=4.4 Hz, 3H), 2.90-2.84 (m, 2H), 2.34-2.26 (m, 2H), 2.12-2.07 (m, 1H), 2.02 (s, 3H), 0.94-0.80 (m, 4H).

### Example 735: Synthesis of N-(5-((5-((1-acetylazetidin-3-yl)oxy)pyridin-2-yl)ethynyl)-8-(methylamino)-2,7-naphthyridin-3-yl)cyclopropanecarboxamide

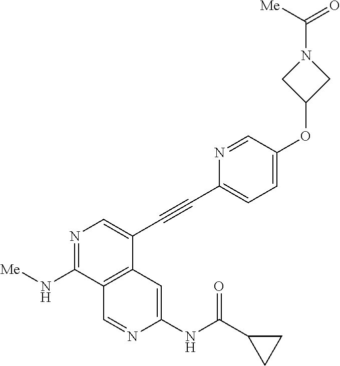 [SMILES: CNc1ncc(C#Cc2ccc(OC3CN(C(C)=O)C3)cn2)c2cc(NC(=O)C3CC3)ncc12]

### Step 1: tert-butyl 3-((6-bromopyridin-3-yl)oxy)azetidine-1-carboxylate

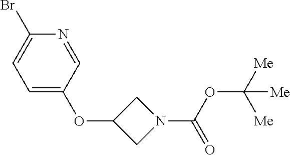 [SMILES: CC(C)(C)OC(=O)N1CC(Oc2ccc(Br)nc2)C1]

A solution of 6-bromopyridin-3-ol (1.00 g; 5.747 mmol; 1.00 eq.), tert-butyl 3-bromoazetidine-1-carboxylate (2.04 g; 8.620 mmol; 1.50 eq.) and Cs₂CO₃ (5.62 g; 17.241 mmol; 3.00 eq.) in DMF (20 mL) was stirred at 1.20° C. for 1 hour. The desired product was detected via LCMS. The resulting mixture was diluted with EtOAc (100 mL), washed with brine (3×10 mL) and dried over anhydrous Na₂SO₄. The organic layer was concentrated under vacuum. The residue was purified by flash chromatography on silica gel column using 0-30% of EtOAc in petroleum ether as eluent to provide tert-butyl 3-((6-bromopyridin-3-yl)oxy)azetidine-1-carboxylate as a white solid (1.89 g, 99.4%). LCMS (ESI) m/z 329.0, [M+H]⁺.

### Step 2: 5-(azetidin-3-yloxy)-2-bromopyridine hydrochloride

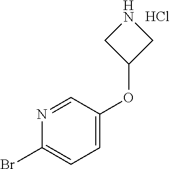 [SMILES: Cl ; Brc1ccc(OC2CNC2)cn1]

To a stirred solution of tert-butyl 3-((6-bromopyridin-3-yl)oxy)azetidine-1-carboxylate (1.00 g; 3.038 mmol; 1.00 eq.) in MeOH (10 mL) was added HCl (gas) (4 M in 1,4-dioxane; 5 mL). The resulting solution was stirred at room temperature for 2 hours. The resulting mixture was concentrated under reduced pressure to provide 5-(azetidin-3-yloxy)-2-bromopyridine hydrochloride as a white solid (866.5 mg, 74.6%). LCMS (ESI) m/z 229.0, [M+H]⁺.

### Step 3: 1-(3-((6-bromopyridin-3-yl)oxy)azetidin-1-yl)ethan-1-one

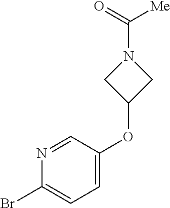 [SMILES: CC(=O)N1CC(Oc2ccc(Br)nc2)C1]

A solution of 5-(azetidin-3-yloxy)-2-bromopyridine hydrochloride (766.0 mg; 2.885 mmol; 1.00 eq.), acetic anhydride (588.9 mg; 5.770 mmol; 2.00 eq.) and DIEA (1.12 g; 8.655 mmol; 3.00 eq.) in CH₂Cl₂ (15 mL). The reaction mixture was stirred at room temperature for 1.5 hours. The desired product was detected via LCMS. The resulting mixture was diluted with EtOAc (50 mL), washed with brine (3×20 mL) and dried over anhydrous Na₂SO₄. The organic layer was concentrated under vacuum. The residue was purified by flash chromatography on silica gel column using 0-10% of MeOH in CH₂Cl₂ to provide 1-(3-((6-bromopyridin-3-yl)oxy)azetidin-1-yl)ethan-1-one as a yellow solid (633.9 mg, 80.3%). LCMS (ESI) m/z 271.0, [M+H]⁺.

### Step 4: N-(5-((5-((1-acetylazetidin-3-yl)oxy)pyridin-2-yl)ethynyl)-8-(methylamino)-2,7-naphthyridin-3-yl)cyclopropanecarboxamide

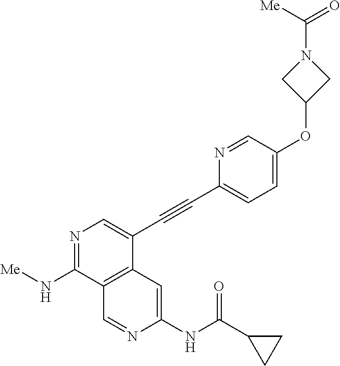 [SMILES: CNc1ncc(C#Cc2ccc(OC3CN(C(C)=O)C3)cn2)c2cc(NC(=O)C3CC3)ncc12]

N-(5-((5-((1-acetylazetidin-3-yl)oxy)pyridin-2-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and i-(3-((6-bromopyridin-3-yl)oxy)azetidin-1-yl)ethan-1-one as the starting material. LCMS (ESI) m/z 457.2, [M+H]⁺. ¹H NMR (400 MHz, DMSO-d₆) δ 11.07 (s, 1H), 9.38 (s, 1H), 8.61 (s, 1H), 8.39-8.35 (m, 1H), 8.31 (s, 1H), 8.27 (d, J=2.4 Hz, 1H), 7.59 (d, J=8.8 Hz, 1H), 7.39-7.36 (m, 1H), 5.19-5.14 (m, 1H), 4.61-4.55 (m, 1H), 4.39-4.28 (m, 1H), 4.18-4.11 (m, 1H), 3.86-3.80 (m, 1H), 3.03 (d, J=4.4 Hz, 3H), 2.11-2.04 (m, 1.1-1), 1.81 (s, 3H), 0.88-0.84 (m, 4H).

### Example 736: Synthesis of N-(5-((5-((1-acetylazetidin-3-yl)oxy)pyridin-2-yl)ethynyl)-8-(methylamino)-2,7-naphthyridin-3-yl)-1-fluorocyclopropane-1-carboxamide

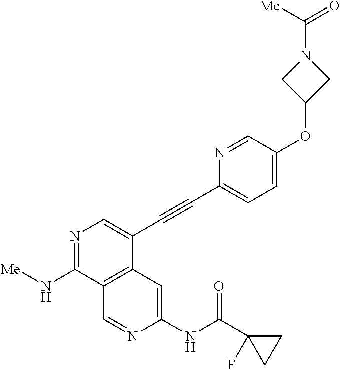 [SMILES: CNc1ncc(C#Cc2ccc(OC3CN(C(C)=O)C3)cn2)c2cc(NC(=O)C3(F)CC3)ncc12]

### Step 1: 4-((5-(azetidin-3-yloxy)pyridin-2-yl)ethynyl)-N¹-methyl-2,7-naphthyridine-1,6-diamine

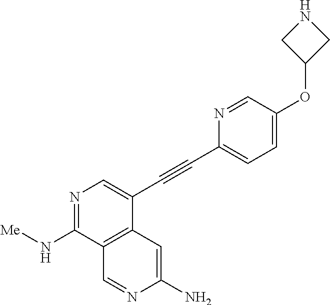 [SMILES: CNc1ncc(C#Cc2ccc(OC3CNC3)cn2)c2cc(N)ncc12]

To a solution of N-(5-((5-((1-acetylazetidin-3-yl)oxy)pyridin-2-yl)ethynyl)-8-(methylamino)-2,7-naphthyridin-3-yl)cyclopropanecarboxamide (Example 735) (180.0 mg; 0.395 mmol; 1.00 eq.) in a mixture solvent of DMSO/MeOH (1:5, 1.2 mL) was added a solution of NaOH (158.0 mg; 3.950 mmol; 10.00 eq.) in water (0.3 mL). The resulting solution was stirred at 60° C. for 14 hours. The desired product was detected via LCMS. The resulting mixture was concentrated under vacuum. The residue was purified by flash chromatography on pre-packed C18 column using 40-60% of MeOH in water (10 mmol/L NH₄HCO₃) as eluent to provide 4-((5-(azetidin-3-yloxy)pyridin-2-yl)ethynyl)-N¹-methyl-2,7-naphthyridine-1,6-diamine as a yellow solid (132.6 mg, 97.1%). LCMS (ESI) m/z 347.1, [M+H]⁺.

### Step 2: 1-(3-((6-((6-amino-1-(methylamino)-2,7-naphthyridin-4-yl)ethynyl)pyridin-3-yl)oxy)azetidin-1-yl)ethan-1-one

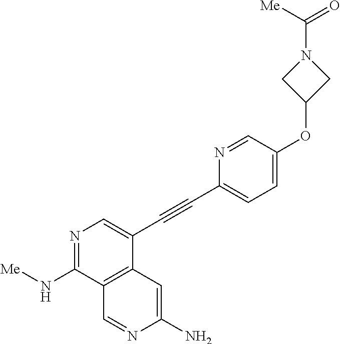 [SMILES: CNc1ncc(C#Cc2ccc(OC3CN(C(C)=O)C3)cn2)c2cc(N)ncc12]

A solution of 4-((5-(azetidin-3-yloxy)pyridin-2-yl)ethynyl)-N¹-methyl-2,7-naphthyridine-1,6-diamine (116.0 mg; 0.335 mmol; 1.00 eq.), acetic anhydride (34.1 mg; 0.335 mmol; 1.00 eq.) and DIEA (129.8 mg; 1.005 mmol; 3.00 eq.) in a mixture solvent of DMF/CH₂Cl₂ (1:7, 4 mL) was stirred at room temperature for 2 hours. The desired product was detected via LCMS. The resulting mixture was concentrated under vacuum. The residue was purified by flash chromatography on silica gel column using 0-15% of MeOH in CH₂Cl₂ as eluent to provide 1-(3-((6-((6-amino-1-(methylamino)-2,7-naphthyridin-4-yl)ethynyl)pyridin-3-yl)oxy)azetidin-1-yl)ethan-1-one as a yellow solid (98.1 mg, 74.0%). LCMS (ESI) m/z 389.2, [M+H]⁺.

### Step 3: N-(5-((5-((1-acetylazetidin-3-yl)oxy)Pyridin-2-yl)ethynyl)-8-(methylamino)-2,7-naphthyridin-3-yl)-1-fluorocyclopropane-1-carboxamide

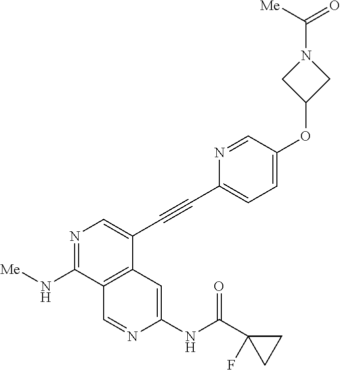 [SMILES: CNc1ncc(C#Cc2ccc(OC3CN(C(C)=O)C3)cn2)c2cc(NC(=O)C3(F)CC3)ncc12]

To a stirred solution of 1-(3-((6-((6-amino-1-(methylamino)-2,7-naphthyridin-4-yl)ethynyl)pyridin-3-yl)oxy)azetidin-1-yl)ethan-1-one (88.00 mg; 0.227 mmol; 1.00 eq.) and 1-fluorocyclopropane-1-carboxylic acid (23.58 mg; 0.227 mmol; 1.00 eq.) in pyridine (4 mL) was added POCl₃ (104.21 mg; 0.681 mmol; 3.00 eq.) dropwise at 0° C. The resulting solution was stirred at room temperature for 10 minutes under nitrogen atmosphere. The desired product was detected via LCMS. The resulting mixture was diluted with EtOAc (40 mL), washed with water (1×10 mL) and dried over anhydrous Na₂SO₄. The organic layer was concentrated under vacuum. The residue was purified by flash chromatography on silica gel column using 5-10% of MeOH in CH₂Cl₂ to provide the product. The crude product was repurified by flash chromatography on pre-packed C18 column using 50-70% of MeOH in water. (10 mmol/L NH₄HCO₃) as eluent to provide N-(5-((5-((1-acetylazetidin-3-yl)oxy)pyridin-2-yl)ethynyl)-8-(methylamino)-2,7-naphthyridin-3-yl)-1-fluorocyclopropane-1-carboxamide as a yellow solid (34.5 mg, 32.0%). LCMS (ESI) m/z 475.2, [M+H]⁺. ¹H NMR (400 MHz, DMSO-d₆) δ 10.49 (s, 1H), 9.43 (s, 1H), 8.60 (s, 1H), 8.49-8.46 (m, 1H), 8.36 (s, 1H), 8.28 (d, J=2.8 Hz, 1H), 7.60 (d, J=8.4 Hz, 1H), 7.39-7.36 (m, 1H), 5.18-5.15 (m, 1H), 4.61-4.57 (m, 1H), 4.34-4.30 (m, 1H), 4.17-4.13 (m, 1H), 3.84-3.80 (m, 1H), 3.05 (d, J=4.4 Hz, 3H), 1.81 (s, 3H), 1.81-1.38 (m, 4H).

### Example 737: Synthesis of N-(5-((4-(2-methoxyacetamido)phenyl)ethynyl)-8-(methylamino)-2,7-naphthyridin-3-yl)cyclopropanecarboxamide

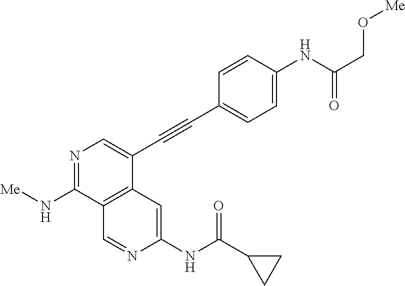 [SMILES: CNc1ncc(C#Cc2ccc(NC(=O)COC)cc2)c2cc(NC(=O)C3CC3)ncc12]

### Step 1: N-(4-bromophenyl)-2-methoxyacetamide

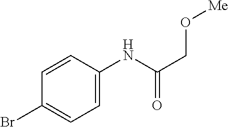 [SMILES: COCC(=O)Nc1ccc(Br)cc1]

To a stirred solution of 4-bromoaniline (200.3 mg; 1.163 mmol; 1.00 eq.) and pyridine (185.1 mg; 2.339 mmol; 2.00 eq.) in THF (2 mL) was added 2-methoxyacetyl chloride (139.3 mg; 1.281 mmol; 1.10 eq.) dropwise at 0° C. The resulting mixture was stirred at 0° C. for 1 hour. The desired product was detected via LCMS. The resulting mixture was diluted with water (10 mL) and extracted with EtOAc (3×10 mL). The combined organic layers were washed with brine (3×10 mL) and dried over anhydrous Na₂SO₄. After filtration, the filtrate was concentrated under reduced pressure to afford N-(4-bromophenyl)-2-methoxyacetamide as a yellow solid (215.2 mg, 75.7%). LCMS (ESI) m/z 244.0, [M+H]⁺.

### Step 2: N-(5-((4-(2-methoxyacetamido)phenyl)ethynyl)-8-(methylamino)-2,7-naphthyridin-3-yl)cyclopropanecarboxamide

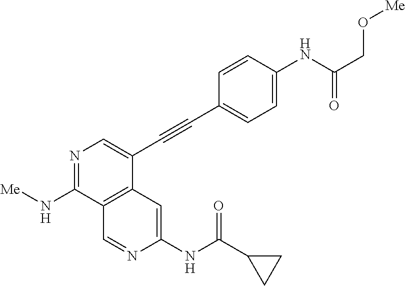 [SMILES: CNc1ncc(C#Cc2ccc(NC(=O)COC)cc2)c2cc(NC(=O)C3CC3)ncc12]

N-(5-((4-(2-methoxyacetamido)phenyl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and N-(4-bromophenyl)-2-methoxyacetamide as the starting material. LCMS (ESI) m/z 430.2, [M+H]⁺. ¹H NMR (400 MHz, DMSO-d₆) δ 11.07 (s, 1H), 9.95 (s, 1H), 9.37 (s, 1H), 8.65 (s, 1H), 8.35-8.29 (m, 1H), 8.26 (s, 1H), 7.81-7.75 (m, 2H), 7.58-7.50 (m, 2H), 4.04 (s, 2H), 3.40 (s, 3H), 3.02 (d, J=4.4 Hz, 3H), 2.13-2.04 (m, 1H), 0.94-0.81 (m, 4H).

### Example 738: Synthesis of N-(5-((5-(dimethylphosphoryl)pyridin-2-yl)ethynyl)-8-(methylamino)-2,7-naphthyridin-3-yl)cyclopropanecarboxamide

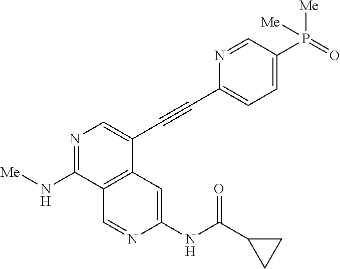 [SMILES: CNc1ncc(C#Cc2ccc(P(C)(C)=O)cn2)c2cc(NC(=O)C3CC3)ncc12]

Step 1: (6-chloropyridin-3-yl)dimethylphosphine oxide

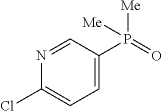 [SMILES: CP(C)(=O)c1ccc(Cl)nc1]

To a stirred solution of hydroxy dimethylphosphane (179.1 mg; 2.293 mmol; 1.10 eq.) and 2-chloro-5-iodopyridine (500.5 mg; 2.088 mmol; 1.00 eq.) in 1,4-dioxane (5 mL) were added Pd₂(dba)₃ (191.5 mg; 0.209 mmol; 0.10 eq.), XantPhos (242.5 mg; 0.418 mmol; 0.20 eq.) and K₃PO₄ (488.1 mg; 2.299 mmol; 1.10 eq.) under nitrogen atmosphere. The resulting mixture was stirred at 100° C. for 14 hours. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to afford (6-chloropyridin-3-yl)dimethylphosphine oxide as a brown solid (120.3 mg, 30.3%). LCMS (ESI) m/z 190.0, [M+H]⁺.

### Step 2: N-(5-((5-(dimethylphosphoryl)pyridin-2-yl)ethynyl)-8-(methylamino)-2,7-naphthyridin-3-yl)cyclopropanecarboxamide

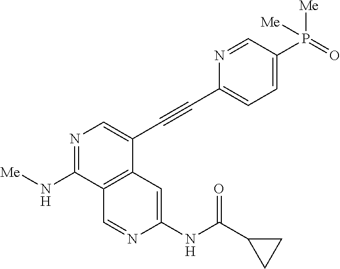 [SMILES: CNc1ncc(C#Cc2ccc(P(C)(C)=O)cn2)c2cc(NC(=O)C3CC3)ncc12]

N-(5-((5-(dimethylphosphoryl)pyridin-2-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and (6-chloropyridin-3-yl)dimethylphosphine oxide as the starting material. LCMS (ESI) m/z 420.2, [M+H]⁺. ¹H NMR (400 MHz, DMSO-d₆) δ 11.12 (s, 1H), 9.40 (s, 1H), 8.96-8.89 (m, 1H), 8.65 (s, 1H), 8.54-8.47 (m, 1H), 8.40 (s, 1H), 8.26-8.18 (m, 1H), 7.75 (d, J=7.6 Hz, 1H), 3.05 (d, J=4.4 Hz, 3H), 2.14-2.05 (m, 1H), 1.77 (s, 3H), 1.74 (s, 3H), 0.95-0.81 (m, 4H).

### Example 739: Synthesis of N-(5-((2-(difluoromethyl)-1-methyl-1H-benzo[d]imidazol-6-yl)ethynyl)-8-(methylamino)-2,7-naphthyridin-3-yl)cyclopropanecarboxamide

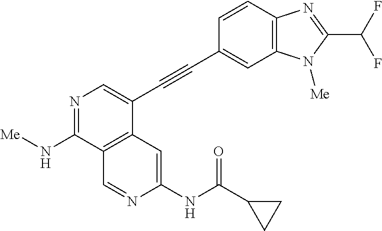 [SMILES: CNc1ncc(C#Cc2ccc3nc(C(F)F)n(C)c3c2)c2cc(NC(=O)C3CC3)ncc12]

### Step 1: 6-bromo-2-(difluoromethyl)-1-methyl-1H-benzo[d]imidazole

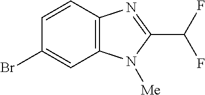 [SMILES: Cn1c(C(F)F)nc2ccc(Br)cc21]

A solution of 5-bromo-M-methylbenzene-1,2-diamine (300.0 mg; 1.492 mmol; 1.00 eq.) and 2,2-difluoroacetic acid (143.2 mg; 1.492 mmol; 1.00 eq.) in HCl (3 M in water, 4 mL) was stirred at 100° C. for 1.4 hours. The desired product was detected via LCMS. The resulting mixture was neutralized to pH=7 with NaOH solution (2 M in water). The resulting mixture was extracted with CH₂Cl₂ (3×15 mL). The combined organic layers were washed with brine (3×3 mL), dried over anhydrous Na₂SO₄, and concentrated under vacuum. The residue was purified by flash chromatography on silica gel column using 0-30% of EtOAc in petroleum ether as eluent to provide 6-bromo-2-(difluoromethyl)-1-methyl-1H-benzo[d]imidazole as a white solid (321.0 mg, 81.8%). LCMS (ESI) m/z 261.0, [M+H]⁺.

### Step 2: N-(5-((2-(difluoromethyl)-1-methyl-1H-benzo[d]imidazol-6-yl)ethynyl)-8-(methylamino)-2,7-naphthyridin-3-yl)cyclopropanecarboxamide

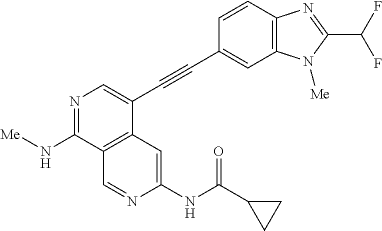 [SMILES: CNc1ncc(C#Cc2ccc3nc(C(F)F)n(C)c3c2)c2cc(NC(=O)C3CC3)ncc12]

N-(5-((2-(difluoromethyl)-1-methyl-1H-benzo[d]imidazol-6-yl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 6-bromo-2-(difluoromethyl)-1-methyl-1H-benzo[d]imidazole as the starting material. LCMS (ESI) m/z 447.2, [M+H]⁺. ¹H NMR (400 MHz, DMSO-d₆) δ 11.13 (s, 1H), 9.39 (s, 1H), 8.80 (s, 1H), 8.36-8.33 (m, 1H), 8.30 (s, 1H), 8.06 (s, 1H), 7.80 (d, J=8.4 Hz, 1H), 7.50-7.45 (m, 1H), 7.44 (t, J=52.2 Hz, 1H), 3.99 (s, 3H), 3.03 (d, J=4.4 Hz, 3H), 2.13-2.07 (m, 1H), 0.93-0.86 (m, 4H).

### Example 740: Synthesis of N-(5-((2-cyclopropyl-1-methyl-1H-benzo[d]imidazol-6-yl)ethynyl)-8-(methylamino)-2,7-naphthyridin-3-yl)cyclopropanecarboxamide

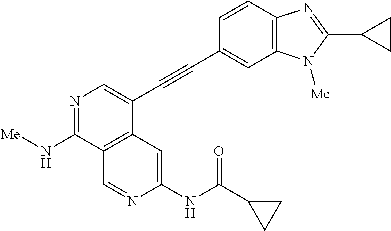 [SMILES: CNc1ncc(C#Cc2ccc3nc(C4CC4)n(C)c3c2)c2cc(NC(=O)C3CC3)ncc12]

### Step 1: 6-bromo-2-cyclopropyl-1-methyl-1H-benzo[d]imidazole

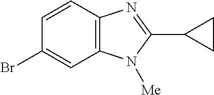 [SMILES: Cn1c(C2CC2)nc2ccc(Br)cc21]

A solution of 5-bromo-N¹-methylbenzene-1,2-diamine (200.0 mg; 0.995 mmol; 1.00 eq.) and cyclopropanecarboxylic acid (428.1 mg; 4.975 mmol; 5.00 eq.) in HCl (3 M in water, 4 mL) was stirred at 100° C. for 14 hours. The desired product was detected via LCMS. The resulting mixture was neutralized to pH=7 with NaOH solution (2 M in water). The resulting mixture was extracted with CH₂Cl₂ (3×15 mL). The combined organic layers were washed with brine (3×3 mL), dried over anhydrous Na₂SO₄, and concentrated under vacuum. The residue was purified by flash chromatography on silica gel column using 0-40% of EtOAc in petroleum ether as eluent to provide 6-bromo-2-cyclopropyl-1-methyl-1H-benzo[d]imidazole as a pink solid (82.5 mg, 32.5%). LCMS (ESI) m/z 251.0, [M+H]⁺.

### Step 2: N-(5-((2-cyclopropyl-1-methyl-1H-benzo[d]imidazol-6-yl)ethynyl)-8-(methylamino)-2,7-naphthyridin-3-yl)cyclopropanecarboxamide

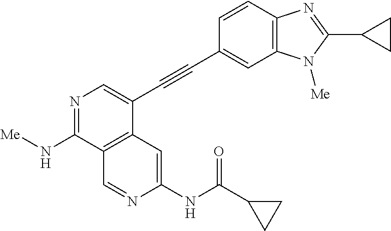 [SMILES: CNc1ncc(C#Cc2ccc3nc(C4CC4)n(C)c3c2)c2cc(NC(=O)C3CC3)ncc12]

N-(5-((2-cyclopropyl-1-methyl-1H-benzo[d]imidazol-6-yl)ethynyl)-8-(methylamino)-2,7-naphthyridin-3-yl)cyclopropanecarboxamide was synthesized using a similar procedure that was previously described in Example 595, by using NV (5-ethynyl-8-(methylamino)-2,7-naphthyridin-3-yl)cyclopropanecarboxamide and 6-bromo-2-cyclopropyl-1-methyl-1H-benzo[d]imidazole as the starting material. LCMS (ESI) m/z 437.2, [M+H]⁺. ¹H NMR (400 MHz, DMSO-d₆) δ 11.11 (s, 1H), 9.38 (s, 1H), 8.79 (s, 1H), 8.31-8.26 (m, 2H), 7.83 (s, 1H), 7.50 (d, J=8.4 Hz, 1H), 7.31 (dd, J=8.4, 1.6 Hz, 1H), 3.90 (s, 3H), 3.03 (d, J=4.4 Hz, 3H), 2.84-2.26 (m, 1H), 2.12-2.08 (m, 1H), 1.13-1.04 (m, 4H), 0.93-0.83 (m, 4H).

### Example 741: Synthesis of (R)—N-(5-((4-((1,4-dioxan-2-yl)methoxy)phenyl)ethynyl)-8-(methylamino)-2,7-naphthyridin-3-yl)cyclopropanecarboxamide

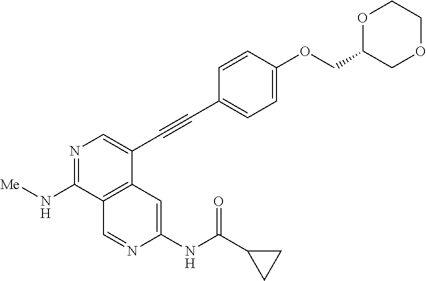 [SMILES: CNc1ncc(C#Cc2ccc(OC[C@H]3COCCO3)cc2)c2cc(NC(=O)C3CC3)ncc12]

### Step 1: (R)-2-((4-bromophenoxy)methyl)-1,4-dioxane

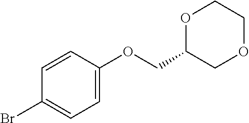 [SMILES: Brc1ccc(OC[C@H]2COCCO2)cc1]

To a stirred solution of 4-bromophenol (1.08 g; 5.780 mmol; 1.00 eq.), PPh₃ (3.01 g; 11.444 mmol; 1.98 eq.) and (S)-(1,4-dioxan-2-yl)methanol (1.36 g; 11.597 mmol; 2.01 eq.) in THF (15 mL) was added DIAD (2.34 g; 11.387 mmol; 1.97 eq.) at 0° C. under nitrogen atmosphere. The resulting solution was stirred at room temperature for 3 hours. The solvent was concentrated under vacuum. The residue was purified by flash chromatography on silica gel column using 25-35% of EtOAc in petroleum ether as eluent to provide (R)-2-((4-bromophenoxy)methyl)-1,4-dioxane as a colorless oil (1.21 g, 76.5%).

### Step 2: (R)—N-(5-((4-((1,4-dioxan-2-yl)methoxy)phenyl)ethynyl)-8-(methylamino)-2,7-naphthyridin-3-yl)cyclopropanecarboxamide

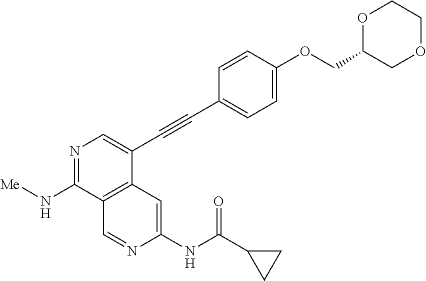 [SMILES: CNc1ncc(C#Cc2ccc(OC[C@H]3COCCO3)cc2)c2cc(NC(=O)C3CC3)ncc12]

(R)—N-(5-((4-((1,4-dioxan-2-yl)methoxy)phenyl)ethynyl)-8-(methylamino)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and (R)-2-((4-bromophenoxy)methyl)-1,4-dioxane as the starting material. LCMS (ESI) m/z 459.2, [M+H]⁺. ¹H NMR (400 MHz, DMSO-d₆) δ 11.07 (s, 1H), 9.37 (s, 1H), 8.64 (s, 1H), 8.31-8.28 (m, 1H), 8.25 (s, 1H), 7.49 (d, J=8.8 Hz, 2H), 7.02 (d, J=8.8 Hz, 2H), 4.01 (d, J=5.2 Hz, 2H), 3.92-3.74 (m, 3H), 3.72-3.60 (m, 2H), 3.57-3.48 (m, 1H), 3.47-3.38 (m, 1H), 3.01 (d, J=4.4 Hz, 3H), 2.13-2.06 (m, 1H), 0.94-0.81 (m, 4H).

### Example 742: Synthesis of (1S,2R)—N-(5-((5-methoxypyridin-2-yl)ethynyl)-8-(methylamino)-2,7-naphthyridin-3-yl)-2-methylcyclopropane-1-carboxamide

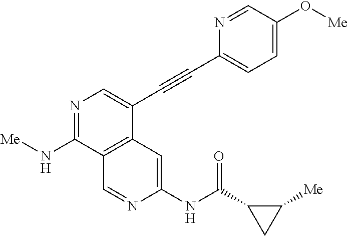 [SMILES: CNc1ncc(C#Cc2ccc(OC)cn2)c2cc(NC(=O)[C@H]3C[C@H]3C)ncc12]

### Step 1: 4-((5-methoxypyridin-2-yl)ethynyl)-N¹-methyl-2,7-naphthyridine-1,6-diamine

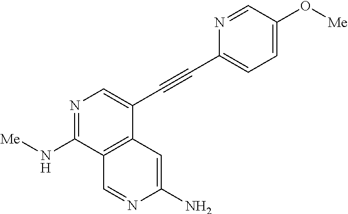 [SMILES: CNc1ncc(C#Cc2ccc(OC)cn2)c2cc(N)ncc12]

To a solution of N-(5-((5-methoxypyridin-2-yl)ethynyl)-8-(methylamino)-2,7-naphthyridin-3-yl)cyclopropanecarboxamide (2.70 g; 7.231 mmol; 1.00 eq.) in DMSO (10 mL) were added MeOH (15 mL) and a solution of NaOH (2.90 g; 72.310 mmol; 10.00 eq.) in H₂O (50 mL) dropwise at room temperature. The resulting mixture was stirred at 60° C. overnight. After MeOH: was removed under reduced pressure, the precipitated solid was collected by filtration. The solid was washed with water (3×1.00 mL) and dried under reduced pressure to afford 4-((5-methoxypyridin-2-yl)ethynyl)-N¹-methyl-2,7-naphthyridine-1,6-diamine (1.60 g, 70.3%) as a brown solid. LCMS (ESI) m/z 306.1, [M+H]⁺.

### Step 2: (1S,2R)—N-(5-((5-methoxypyridin-2-yl)ethynyl)-8-(methylamino)-2,7-naphthyridin-3-yl)-2-methylcyclopropane-1-carboxamide

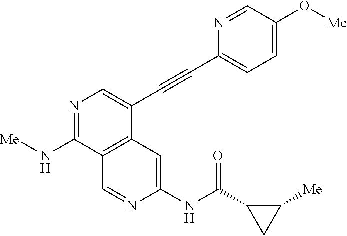 [SMILES: CNc1ncc(C#Cc2ccc(OC)cn2)c2cc(NC(=O)[C@H]3C[C@H]3C)ncc12]

To a solution of 4-((5-methoxypyridin-2-yl)ethynyl)-N¹-methyl-2,7-naphthyridine-1,6-diamine (400.0 mg; 1.310 mmol; 1.00 eq.) and (1S,2R)-2-methylcyclopropane-1-carboxylic acid (118.0 mg; 1.179 mmol; 0.90 eq.) in pyridine (8 mL) was added POCl₃ (602.5 mg; 3.930 mmol; 3.00 eq.) dropwise at 0° C. under nitrogen atmosphere. The mixture was stirred at room temperature for 0.5 hours. The reaction was quenched by the addition of water (2 mL) at 0° C. The solvent was concentrated under vacuum. The residue was purified by flash chromatography on silica gel column using 0-20% of MeOH in CH₂Cl₂ as eluent to provide the crude product. The crude product was purified by recrystallization with dichloromethane/methanol (1:1, 30 mL) to afford (1S,2R)—N-(5-((5-methoxypyridin-2-yl)ethynyl)-8-(methylamino)-2,7-naphthyridin-3-yl)-2-methylcyclopropane-1-carboxamide as a yellow solid (183.2 mg, 35%). LCMS (ESI) m/z 388.2, [M+H]⁺. ¹H NMR (400 MHz, DMSO-d₆) δ 10.97 (s, 1H), 9.37 (s, 1H), 8.63 (s, 1H), 8.40-8.29 (m, 3H), 7.62 (d, J=8.8 Hz, 1H), 7.47 (dd, J=8.8, 3.2 Hz, 1H), 3.89 (s, 3H), 3.03 (d, J=4.4 Hz, 3H), 2.15-2.04 (m, 1H), 1.39-1.25 (m, 1H), 1.15 (d, J=6.4 Hz, 3H), 1.04-0.96 (m, 1H), 0.91-0.82 (m, 1H).

### Example 743: Synthesis of 1-fluoro-N-(5-((5-methoxypyridin-2-yl)ethynyl)-8-(methylamino)-2,7-naphthyridin-3-yl)cyclopropane-1-carboxamide

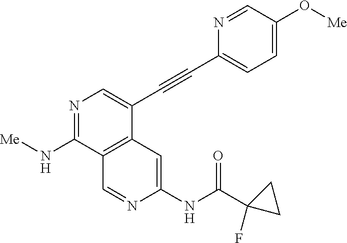 [SMILES: CNc1ncc(C#Cc2ccc(OC)cn2)c2cc(NC(=O)C3(F)CC3)ncc12]

1-Fluoro-N-(5-((5-methoxypyridin-2-yl)ethynyl)-8-(methylamino)-2,7-naphthyridin-3-yl)cyclopropane-1-carboxamide was synthesized using a similar procedure that was previously described in example 742 by using 4-((5-methoxypyridin-2-yl)ethynyl)-N¹-methyl-2,7-naphthyridine-1,6-diamine and 1-fluorocyclopropane-1-carboxylic acid as the starting material. LCMS (ESI) m/z 392.1, [M+H]⁺. ¹H NMR (400 MHz, DMSO-d₆) δ 10.49 (s, 1H), 9.43 (s, 1H), 8.60 (s, 1H), 8.50-8.44 (m, 1H), 8.39-8.32 (m, 2H), 7.60 (d, J=8.4 Hz, 1H), 7.50-7.45 (m, 1H), 3.88 (s, 3H), 3.04 (d, J=4.4 Hz, 3H), 1.55-1.40 (m, 4H).

### Examples 744 and 745: Synthesis of (1R,18)-2-(difluoromethyl)-N-(5-((5-methoxypyridin-2-yl)ethynyl)-8-(methylamino)-2,7-naphthyridin-3-yl)cyclopropane-1-carboxamide (Example 744) and (1S,2R)-2-(difluoromethyl)-N-(5-((5-methoxypyridin-2-yl)ethynyl)-8-(methylamino)-2,7-naphthyridin-3-yl)cyclopropane-1-carboxamide (Example 745)

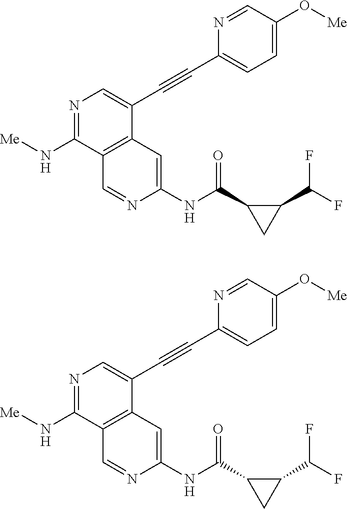 [SMILES: CNc1ncc(C#Cc2ccc(OC)cn2)c2cc(NC(=O)[C@@H]3C[C@@H]3C(F)F)ncc12 ; CNc1ncc(C#Cc2ccc(OC)cn2)c2cc(NC(=O)[C@H]3C[C@H]3C(F)F)ncc12]

To a solution of 4-((5-methoxypyridin-2-yl)ethynyl)-N¹-methyl-2,7-naphthyridine-1,6-diamine (1.60 g; 5.240 mmol; 1.00 eq.) and 2-(difluoromethyl)cyclopropane-1-carboxylic acid (cis racemate) (0.61 g; 4.716 mmol; 0.90 eq.) in pyridine (20 mL) was added POCl₃ (2.40 g; 15.720 mmol; 3.00 eq.) dropwise at 0° C. The resulting mixture was stirred at room temperature for 1 hour. The desired product was detected via LCMS. The solution was quenched with water (30 mL) and extracted with EtOAc (3×100 mL). The combined organic layers were washed with brine (1×50 mL) and dried over anhydrous Na₂SO₄. After filtration, the filtrate was concentrated under reduced pressure. The residue was purified by flash chromatography on silica gel column using 5-10% of MeOH in CH₂Cl₂ as eluent to afford the cis racemate. The cis racemate was separated by Chiral-HPLC (Column: CHIRALPAK IG, 2×25 cm, 5 μm; Mobile Phase A: Hex (0.5% 2 M NH₃-MeOH), Mobile Phase B: MeOH:DCM=1:1; Flow rate: 20 mL/min; Gradient: 50% B to 50% B in 15 min; Wave Length: 220/254 nm; R_{T1}(min): 7.67; R_{T2}(min): 12.63) to afford (1R,2S)-2-(difluoromethyl)-N-(54(5-methoxypyridin-2-yl)ethynyl)-8-(methylamino)-2,7-naphthyridin-3-yl)cyclopropane-1-carboxamide (Example 744, the faster peak) as a yellow solid (423.4 mg, 18.8%) and (1S,2R)-2-(difluoromethyl)-N-(5-(5-methoxypyridin-2-yl)ethynyl)-8-(methylamino)-2,7-naphthyridin-3-yl)cyclopropane-1-carboxamide (Example 745, the slower peak) as a yellow solid (418.2 mg, 18.7%). The two configurations are arbitrarily assigned. LCMS (ESI) m/z 424.2 [M+H]⁺.

HMNR for Example 744: ¹H NMR (400 MHz, DMSO-d₆) δ 11.32 (s, 1H), 9.40 (s, 1H), 8.59 (s, 1H), 8.48-8.38 (m, 1H), 8.33-8.32 (m, 2H), 7.62 (d, J=8.8 Hz, 1H), 7.58-7.38 (m, 1H), 6.40-5.75 (m, 1H), 3.89 (s, 3H), 3.03 (d, J=4.4 Hz, 3H), 2.48-2.38 (m, 1H), 2.03-1.82 (m, 1H), 1.40-1.28 (m, 2H).

HNMR for Example 745: ¹H NW, (400 MHz, DMSO-d₆) δ 11.32 (s, 1H), 9.40 (s, 1H), 8.59 (s, 1H), 8.48-8.38 (m, 1H), 8.33-8.32 (m, 2H), 7.62 (d, J=8.8 Hz, 1H), 7.58-7.38 (m, 1H), 6.40-5.75 (m, Ft), 3.89 (s, 3H), 3.03 (d, J=4.4 Hz, 3H), 2.48-2.38 (m, 1H), 2.03-1.82 (m, 1H), 1.40-1.28 (m, 2H).

### Examples 746 and 747: Synthesis of (1R,2S)—N-(5-((5-methoxypyridin-2-yl)ethynyl)-8-(methylamino)-2,7-naphthyridin-3-yl)-2-(trifluoromethyl)cyclopropane-1-carboxamide (Example 746) and (1S,2R)—N-(5-((5-methoxypyridin-2-yl)ethynyl)-8-(methylamino)-2,7-naphthyridin-3-yl)-2-(trifluoromethyl)cyclopropane-1-carboxamide (Example 747)

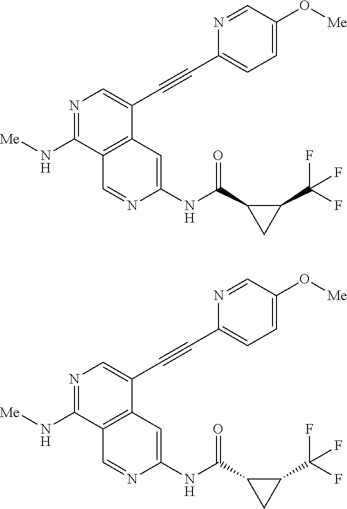 [SMILES: CNc1ncc(C#Cc2ccc(OC)cn2)c2cc(NC(=O)[C@@H]3C[C@@H]3C(F)(F)F)ncc12 ; CNc1ncc(C#Cc2ccc(OC)cn2)c2cc(NC(=O)[C@H]3C[C@H]3C(F)(F)F)ncc12]

To a solution of 4-((5-methoxypyridin-2-yl)ethynyl)-N¹-methyl-2,7-naphthyridine-1,6-diamine (150.0 mg; 0.491 mmol; 1.00 eq.) and 2-(trifluoromethyl)cyclopropane-1-carboxylic acid (cis racemate) (68.2 mg; 0.442 mmol; 0.90 eq.) in pyridine (6 mL) was added POCl₃ (225.9 mg; 1.473 mmol; 3.00 eq.) dropwise at 0° C. The resulting mixture was stirred at room temperature for 1 hour under nitrogen atmosphere. The desired product was detected via LCMS. The reaction was quenched by the addition of water. (10 mL) and extracted with EtOAc (3×50 mL). The combined organic layers were washed with brine (1×20 mL) and dried over anhydrous Na₂SO₄. After filtration, the filtrate was concentrated under reduced pressure. The residue was purified by flash chromatography on silica gel column using 2-10% of MeOH in CH₂Cl₂ as eluent to afford the cis racemate. The cis racemate was separated by Chiral-HPLC (Column: Chiral ART Cellulose-SA, 2×25 cm, 5 μm; Mobile Phase A: Hex (0.5% 2 M NT-13-MeOH), Mobile Phase B: EtOH:DCM=1:1; Flow rate: 20 mL/min; Gradient: 30% B to 30% B in 21 min; Wave Length: 220/254 nm; R_{T1}(min): 14.64; R_{T2}(min): 19.65) to afford (1R,2S)—N-(5-((5-methoxypyridin-2-yl)ethynyl)-8-(methylamino)-2,7-naphthyridin-3-yl)-2-(trifluoromethyl)cyclopropane-1-carboxamide (Example 746, the faster peak) as a yellow solid (35.9 mg, 25.7%) and (1S,2R)—N-(5-((5-methoxypyridin-2-yl)ethynyl)-8-(methylamino)-2,7-naphthyridin-3-yl)-2-(trifluoromethyl)cyclopropane-1-carboxamide (Example 747, the slower peak) as a yellow solid (30.9 mg, 22.4%). The two configurations are arbitrarily assigned. LCMS (ESI) m/z 442.1 [M+H]⁺.

HNMR for example 746: ¹H NMR (400 MHz, DMSO-d₆) δ 11.24 (s, 1H), 9.39 (s, 1H), 8.59 (s, 1H), 8.42-8.37 (m, 1H), 8.33-8.30 (m, 2H), 7.61 (d, J=8.8 Hz, 1H), 7.50-7.41 (m, 1H), 3.89 (s, 3H), 3.03 (d, J=4.4 Hz, 3H), 2.49-2.42 (m, 1H), 2.32-2.22 (m, 1H), 1.64-1.57 (m, 1H), 1.40-1.30 (m, 1H).

HNMR for example 747: ¹H NMR (400 MHz, DMSO-d₆) δ 11.24 (s, 1H), 9.39 (s, 1H), 8.59 (s, 1H), 8.42-8.37 (m, 1H), 8.33-8.30 (m, 2H), 7.61 (d, J=8.8 Hz, 1H), 7.50-7.41 (m, 1H), 3.89 (s, 3H), 3.03 (d, J=4.4 Hz, 3H), 2.49-2.42 (m, 1H), 2.32-2.22 (m, 1H), 1.64-1.57 (m, 1H), 1.40-1.30 (m, 1H).

### Examples 748 and 749: Synthesis of N-(8-(methylamino)-5-((3-methylbenzo[d]isoxazol-6-yl)ethynyl)-2,7-naphthyridin-3-yl)cyclopropanecarboxamide (Example 748) and N-(5-((4-acetyl-3-hydroxyphenyl)ethynyl)-8-(methylamino)-2,7-naphthyridin-3-yl)cyclopropanecarboxamide (Example 749)

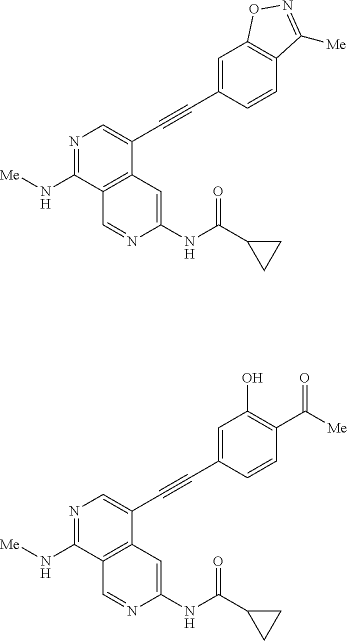 [SMILES: CNc1ncc(C#Cc2ccc3c(C)noc3c2)c2cc(NC(=O)C3CC3)ncc12 ; CNc1ncc(C#Cc2ccc(C(C)=O)c(O)c2)c2cc(NC(=O)C3CC3)ncc12]

To a solution of N-(5-ethynyl-8-(methylamino)-2,7-naphthyridin-3-yl)cyclopropanecarboxamide (70 mg, 0.263 mmol, 1.51 eq.) and 6-bromo-3-methylbenzo[d]isoxazole (37 mg, 0.1.74 mmol, 1 eq.) in DMF (2.0 mL) was added XPhos Pd G₃ (29.7 mg, 0.035 mmol, 0.20 eq.), XPhos (16.7 mg, 0.035 mmol, 0.20 eq.), CuI (6.7 mg, 0.035 mmol, 0.20 eq.) and Et₃N (53.1 mg, 0.525 mmol, 3.01 equiv) under nitrogen atmosphere. The resulting solution was stirred at 90° C. for 12 hours. The desired product was detected via LCMS. The resulting mixture was diluted with EtOAc (20 mL) and washed with brine (3×5 mL). The organic layer was dried over anhydrous Na₂SO₄. After filtration, the filtrate was concentrated under reduced pressure. The residue was purified by flash chromatography on silica gel column using 2-10% of MeOH in CH₂Cl₂ to afford the crude product. The crude product was repurified by reverse phase preparative HPLC (Column: XSelect CSH Prep C18 OBD Column, 19×250 mm, 511 m; gradient elution of 30-63% MeCN in water over a 16 min period, where water contains 0.1% formic acid, flow rate: 20 mL/min, detector UV wavelength: 254 nm) to afford N-(8-(methylamino)-5-((3-methylbenzo[d]isoxazol-6-yl)ethynyl)-2,7-naphthyridin-3-yl)cyclopropanecarboxamide as light yellow solid (5.1 mg, 7.3%) and N-(5-((4-acetyl-3-hydroxyphenyl)ethynyl)-8-(methylamino)-2,7-naphthyridin-3-yl)cyclopropanecarboxamide as light yellow solid (6.7 mg, 9.3%).

Data for Example 748: LCMS (ESI) m/z 398.2, [M+H]⁺. ¹H NMR (400 MHz, DMSO-d₆) δ 11.11 (s, 1H), 9.39 (s, 1H), 8.72 (s, 1H), 8.48-8.40 (m, 1H), 8.34 (s, 1H), 7.93 (d, J=8.0 Hz, 1H), 7.86 (s, 1H), 7.61-7.52 (m, 1H), 3.04 (d, J=4.4 Hz, 3H), 2.59 (s, 3H), 2.15-2.03 (m, 1H), 0.98-0.79 (m, 4H).

Data for Example 749: LCMS (ESI) m/z 401.2, [M+H]⁺. ¹H NMR (400 MHz, DMSO-d₆) δ 12.05 (s, 1H), 11.12 (s, 1H), 9.38 (s, 1H), 8.66 (s, 1H), 8.49-8.40 (m, 1H), 8.34 (s, 1H), 7.94 (d, J=8.0 Hz, 1H), 7.17-7.07 (m, 2H), 3.04 (d, J=4.4 Hz, 3H), 2.66 (5, 3H), 2.16-2.03 (m, 1H), 0.98-0.80 (m, 4H).

### Example 750: Synthesis of N-(5-((3,4-dihydro-1H-benzo[4,5]imidazo[2,1-c][1,4]oxazin-7-yl)ethynyl)-8-(ethylamino)-2,7-naphthyridin-3-yl)cyclopropanecarboxamide

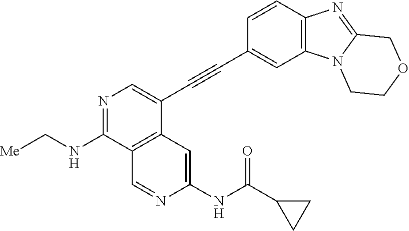 [SMILES: CCNc1ncc(C#Cc2ccc3nc4n(c3c2)CCOC4)c2cc(NC(=O)C3CC3)ncc12]

### Step 1: N-(8-(ethylamino)-5-((trimethylsilyl)ethynyl)-2,7-naphthyridin-3-yl)cyclopropanecarboxamide

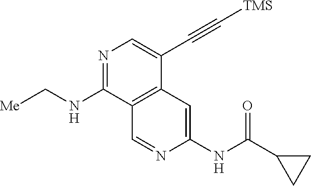 [SMILES: CCNc1ncc(C#C[Si](C)(C)C)c2cc(NC(=O)C3CC3)ncc12]

A solution of N-(5-bromo-8-(ethylamino)-2,7-naphthyridin-3-yl)cyclopropanecarboxamide (Example 450, step 3) (1.00 g; 2.984 mmol; 1.00 eq.), ethynyltrimethylsilane (585.0 mg; 5.966 mmol; 2.00 eq.), Pd(dppf)Cl₂·CH₂Cl₂ (1.21 g; 1.492 mmol; 0.50 eq.), DIEA. (2.31 g; 17.900 mmol; 6.00 eq.) and CuI (283.5 mg; 1.492 mmol; 0.50 eq.) in DMF (10 mL) was stirred at 50° C. for 16 hours under nitrogen atmosphere. The mixture was diluted with water (20 mL) and extracted with EtOAc (3×50 mL). The combined organic layers were washed with brine (2×10 mL), dried over Na₂SO₄. After filtration, the filtrate was concentrated under reduced pressure. The residue was purified by flash chromatography on silica gel column using 0-10% of MeOH in CH₂Cl₂ as eluent to provide N-(8-(ethylamino)-5-((trimethylsilyl)ethynyl)-2,7-naphthyridin-3-yl)cyclopropanecarboxamide as a brown solid (659.4 mg, 62.7%). LCMS (ESI) m/z 353.2, [M+H]⁺.

### Step 2: N-(8-(ethylamino)-5-ethynyl-2,7-naphthyridin-3-yl)cyclopropanecarboxamide

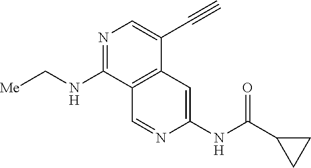 [SMILES: C#Cc1cnc(NCC)c2cnc(NC(=O)C3CC3)cc12]

To a stirred solution of N-(8-(ethylamino)-5-((trimethylsilyl)ethynyl)-2,7-naphthyridin-3-yl)cyclopropanecarboxamide (600.0 mg; 1.702 mmol; 1.00 eq.) in MeOH (20 mL) was added K₂CO₃ (625.1 mg; 4.544 mmol; 2.67 eq.) under nitrogen atmosphere. The mixture was stirred at 20° C. for 1 hour. The mixture was diluted with H₂O (20 mL) and extracted with EtOAc (3×30 mL). The combined organic layers were washed with brine (10 mL) and dried over Na₂SO₄. After filtration, the filtrate was concentrated under reduced pressure. The residue was purified by flash chromatography on silica gel column using 0-10% of MeOH in CH₂Cl₂ as eluent to provide N-(8-(ethylamino)-5-ethynyl-2,7-naphthyridin-3-yl)cyclopropanecarboxamide as a yellow solid (407.1 mg, 83.8%). LCMS (ESI) m/z 281.1, [M+H]⁺.

### Step 3: N-(5-((3,4-dihydro-1H-benzo[4,5]imidazo[2,1-c][1,4]oxazin-7-yl)ethynyl)-8-(ethylamino)-2,7-naphthyridin-3-yl)cyclopropanecarboxamide

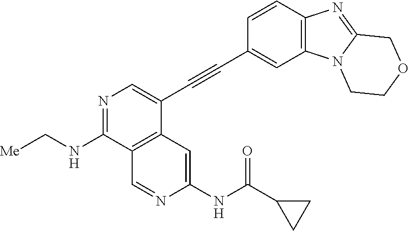 [SMILES: CCNc1ncc(C#Cc2ccc3nc4n(c3c2)CCOC4)c2cc(NC(=O)C3CC3)ncc12]

To a solution of N-(8-(ethylamino)-5-ethynyl-2,7-naphthyridin-3-yl)cyclopropanecarboxamide (149.3 mg; 0.533 mmol; 1.50 eq.) and 7-bromo-3,4-dihydro-1H-benzo[4,5]imidazo[2,1-c][1,4]oxazine (Example 720, Step 3) (90.0 mg; 0.356 mmol; 1.00 eq.) in DMF (4 mL) were added XphosPdG₃ (60.0 mg; 0.071 mmol; 0.20 eq.), Xphos (33.8 mg; 0.071 mmol; 0.20 eq.), Et₃N (107.7 mg; 1.066 mmol; 3.00 eq.) and CuI (13.5 mg; 0.071 mmol; 0.20 eq.) under nitrogen atmosphere. The resulting solution was stirred at 90° C. for 1 hour. The mixture was diluted with water (5 mL) and extracted with EtOAc (3×20 mL). The combined organic layers were washed with brine (2×5 mL), dried over Na₂SO₄. After filtration, the filtrate was concentrated under reduced pressure. The residue was purified by flash chromatography on silica gel column using 0-20% of MeOH in CH₂Cl₂ as eluent to provide N-(5-((3,4-dihydro-1H-benzo[4,5]imidazo[2,1-c][1,4]oxazin-7-yl)ethynyl)-8-(ethylamino)-2,7-naphthyridin-3-yl)cyclopropanecarboxamide as a yellow solid (37.1 mg, 22.5%). LCMS (ESI) m/z 453.2, [M+H]⁺. ¹H NMR (400 MHz, DMSO-d₆) S 1.1.10 (s, 1H), 9.43 (s, 1H), 8.75 (s, 1H), 8.28-8.19 (m, 2H), 7.85 (s, 1H), 7.63 (d, J=8.4 Hz, 1H), 7.41 (dd, J=8.4, 1.2 Hz, 1H), 4.99 (s, 2H), 4.31-4.14 (m, 4H), 3.63-3.53 (m, 2H), 2.15-2.05 (m, 1H), 1.26 (t, J=7.2 Hz, 3H), 0.92-0.80 (m, 4H).

### Example 751: Synthesis of M-methyl-4-((1-methyl-1H-benzo[d][1,2,3]triazol-6-yl)ethynyl)-N⁶-(pyridin-2-yl)-2,7-naphthyridine-1,6-diamine

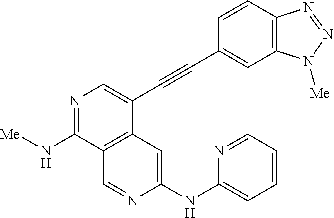 [SMILES: CNc1ncc(C#Cc2ccc3nnn(C)c3c2)c2cc(Nc3ccccn3)ncc12]

### Step 1: N¹-methyl-4-((1-methyl-1H-benzo[d][1,2,3]triazol-6-yl)ethynyl)-2,7-naphthyridine-1,6-diamine

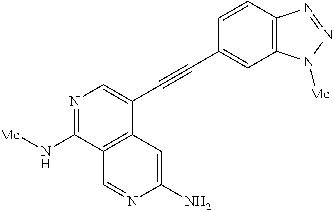 [SMILES: CNc1ncc(C#Cc2ccc3nnn(C)c3c2)c2cc(N)ncc12]

To a stirred solution of N-(5-((1-methyl-1H-benzo[d][1,2,3]triazol-6-yl)ethynyl)-8-(methylamino)-2,7-naphthyridin-3-yl)cyclopropanecarboxamide (Example 595) (180.0 mg; 0.453 mmol; 1.00 eq.) in a mixture solvent of MeOH/DMSO (5:1, 18 mL) was added a solution of NaOH (180.0 mg; 4.500 mmol; 9.94 eq.) in water (5 mL). The resulting solution was stirred at 60° C. for 12 hours. The desired product was detected via LCMS. The mixture was concentrated under vacuum. The residue was purified by flash chromatography on pre-packed C18 column using 40-70% of MeCN in water (10 mmol/L NH₄HCO₃) as eluent to provide N¹-methyl-44(1-methyl-1H-benzo[d][1,2,3]triazol-6-yl)ethynyl)-2,7-naphthyridine-1,6-diamine as a white solid (100 mg, 67.1%). LCMS (ESI) m/z 330.1, [M+H]⁺.

### Step 2: N¹-methyl-4-((1-methyl-1H-benzo[d][1,2,3]triazol-6-yl)ethynyl)-N⁶-(pyridin-2-yl)-2,7-naphthyridine-1,6-diamine

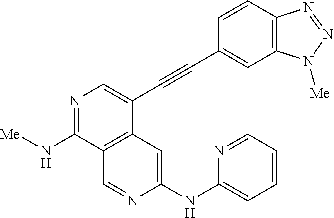 [SMILES: CNc1ncc(C#Cc2ccc3nnn(C)c3c2)c2cc(Nc3ccccn3)ncc12]

To a solution of 2-chloropyridine (35.0 mg; 0.308 mmol, 1.02 eq.) and N¹-methyl-4-((1-methyl-1H-benzo[d][1,2,3]triazol-6-yl)ethynyl)-2,7-naphthyridine-1,6-diamine (100.0 mg; 0.304 mmol; 1.00 eq.) in 1,4-dioxane (8 mL) were added XantPhos (45 mg; 0.078 mmol; 0.26 eq.), Pd₂(dba)₃ (60.0 mg; 0.066 mmol, 0.22 eq.) and Cs₂CO₃(200.0 mg; 0.614 mmol; 2.02 eq.) under nitrogen atmosphere. The resulting solution was stirred at 130° C. for 4 hours. The reaction mixture was concentrated under vacuum. The residue was purified by flash chromatography on silica gel column using 5-10% of MeOH in C1120.2 as eluent to provide N¹-methyl-44(1-methyl-1/1-benzo[d][1,2,3]triazol-6-yl)ethynyl)-N⁶-(pyridin-2-0)-2,7-naphthyridine-1,6-diamine as a yellow solid (8.2 mg, 6.6%). LCMS (ESI) m/z 407.2, [M+H]⁺. ¹H NMR (300 MHz, DMSO-d₆) δ 10.11 (s, 1H), 9.32 (s, 1H), 8.73 (s, 1H), 8.41-8.25 (m, 3H), 8.19-8.13 (m, 1H), 8.09 (s, 1H), 7.76-7.61 (m, 2H), 7.46 (d, J=8.1. Hz 1H), 7.02-6.96 (m, 1H), 4.36 (s, 3H), 3.03 (d, J=4.2 Hz, 3H).

### Example 752: Synthesis of (1S,2S)-2-fluoro-N-(5-((4-methoxyphenyl)ethynyl)-8-(methylamino)-2,7-naphthyridin-3-yl)cyclopropane-1-carboxamide

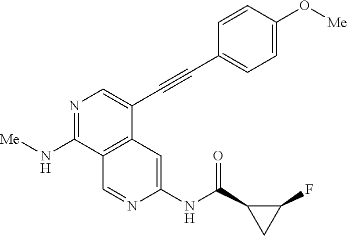 [SMILES: CNc1ncc(C#Cc2ccc(OC)cc2)c2cc(NC(=O)[C@@H]3C[C@@H]3F)ncc12]

### Step 1: 4-((4-methoxyphenyl)ethynyl)-N¹-methyl-2,7-naphthyridine-1,6-diamine

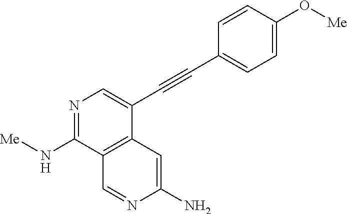 [SMILES: CNc1ncc(C#Cc2ccc(OC)cc2)c2cc(N)ncc12]

To a solution of N-(5-((4-methoxyphenyl)ethynyl)-8-(methylamino)-2,7-naphthyridin-3-yl)cyclopropanecarboxamide (Example 591) (272.0 mg; 0.730 mmol; 1.00 eq.) in a mixture solvent of MeOH/DMSO (5:1, 12 mL) was added a solution of NaOH (292.1 mg; 7.300 mmol; 10.00 eq.) in water (3 mL). The reaction was stirred at 60° C. for 16 hours. The desired product was detected via LCMS. The resulting mixture was concentrated under reduced pressure. The residue was purified by flash chromatography on pre-packed C18 column using 30-90% of MeOH in water (10 mmol/L NH₄HCO₃) as eluent to provide 4-((4-methoxyphenyl)ethynyl)-N¹-methyl-2,7-naphthyridine-1,6-diamine as a yellow solid (131.0 mg, 58.9%). LCMS (ESI) m/z 305.1, [M+H]⁺.

### Step 2: (1S,2S)-2-fluoro-N-(5-((4-methoxyphenyl)ethynyl)-8-(methylanino)-2,7-naphthyridin-3-yl)cyclopropane-1-carboxamide

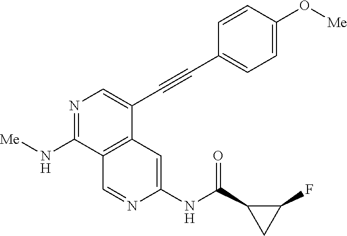 [SMILES: CNc1ncc(C#Cc2ccc(OC)cc2)c2cc(NC(=O)[C@@H]3C[C@@H]3F)ncc12]

To a solution of 4-((4-methoxyphenyl)ethynyl)-N¹-methyl-2,7-naphthyridine-1,6-diamine (80 mg; 0.263 mmol; 1.00 eq.) and (1S,2S)-2-fluorocyclopropane-1-carboxylic acid (24.1 mg; 0.231 mmol; 0.88 eq.) in pyridine (2 mL) was added POCl₃ (120.1 mg; 0.784 mmol; 2.98 eq.) at 0° C. under nitrogen atmosphere. The reaction was stirred at room temperature for 2 hours. The desired product was detected via LCMS. The resulting mixture was diluted with EtOAc (50 mL) and washed with brine (3×5 mL). The organic layer was dried over anhydrous Na₂SO₄. After filtration, the filtrate was concentrated under reduced pressure. The residue was purified by flash chromatography on silica gel column using 0-10% of MeOH in CH₂Cl₂ as eluent to provide (1,5,2)-2-fluoro-N-(5-((4-methoxyphenyl)ethynyl)-8-(methylamino)-2,7-naphthyridin-3-yl)cyclopropane-1-carboxamide as light yellow solid (39.5 mg, 38.4%). LCMS (ESI) m/z 391.0, [M+H]⁺. ¹H NMR (400 MHz, DMSO-d₆) δ 11.11 (s, 1H), 9.38 (s, 1H), 8.65 (s, 1H), 8.32-8.27 (m, 1H), 8.26 (s, 1H), 7.52 (d, J=8.8 Hz, 2H), 7.02 (d, J=8.8 Hz, 2H), 5.14-4.73 (m, 1H), 3.81. (s, 3H), 3.02 (d, J=4.4 Hz, 3H), 2.34-2.23 (m, 1H), 1.78-1.72 (m, 1H), 1.27-1.1.4 (m, 1H).

### Example 753: Synthesis of (1S,2R)—N-(5-((4-methoxyphenyl)ethynyl)-8-(methylamino)-2,7-naphthyridin-3-yl)-2-methylcyclopropane-1-carboxamide

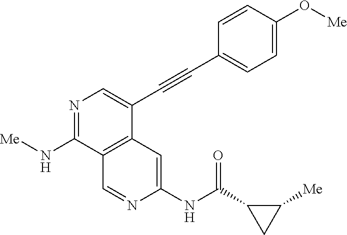 [SMILES: CNc1ncc(C#Cc2ccc(OC)cc2)c2cc(NC(=O)[C@H]3C[C@H]3C)ncc12]

To a solution of 4-((4-methoxyphenyl)ethynyl)-N¹-methyl-2,7-naphthyridine-1,6-diamine (Example 752, Step 1) (80.0 mg; 0.263 mmol; 1.00 eq.) and (1S,2R)-2-methylcyclopropane-1-carboxylic acid (24.0 mg; 0.231 mmol; 0.88 eq.) in pyridine (2 mL) was added POCl₃ (120.1 mg; 0.784 mmol; 2.98 eq.) dropwise at 0° C. The reaction was stirred at room temperature for 2 hours. The desired product was detected via LCMS. The resulting mixture was diluted with EtOAc (50 mL) and washed with brine (3×5 mL). The organic layer was dried over anhydrous Na₂SO₄. The solvent was concentrated under reduced pressure. The residue was purified by flash chromatography on silica gel column using 0-10% of MeOH in CH₂Cl₂ as eluent to provide (1S,2R)—N-(5-((4-methoxyphenyl)ethynyl)-8-(methylamino)-2,7-naphthyridin-3-yl)-2-1.0 methylcyclopropane-1-carboxamide as yellow solid (30.8 mg, 30.3%). LCMS (ESI) m/z 387.1, [M+H]⁺. ¹H NMR (400 MHz, DMSO-d₆) δ 10.97 (s, 1H), 9.36 (s, 1H), 8.67 (s, 1H), 8.32-8.27 (m, 1H), 8.24 (s, 1H), 7.52 (d, J=8.8 Hz, 2H), 7.01 (d, J=8.8 Hz, 2H), 3.81 (s, 3H), 3.02 (d, J=4.4 Hz, 3H), 2.15-2.05 (m, 1H), 1.38-1.24 (m, 1H), 1.16 (d, J=6.0 Hz, 3H), 1.05-0.96 (m, 1H), 0.91-0.83 (m, 1H).

### Examples 754 and 755: Synthesis of (1R,2S)-2-(difluoromethyl)-N-(8-(methylamino)-5-(pyrazolo[1,5-a]pyridin-2-ylethynyl)-2,7-naphthyridin-3-yl)cyclopropane-1-carboxamide (Example 754) and (1S,2R)-2-(difluoromethyl)-N-(8-(methylamino)-5-(pyrazolo[1,5-a]pyridin-2-ylethynyl)-2,7-naphthyridin-3-yl)cyclopropane-1-carboxamide (Example 755)

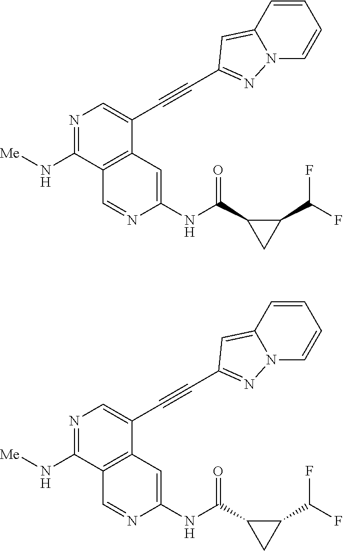 [SMILES: CNc1ncc(C#Cc2cc3ccccn3n2)c2cc(NC(=O)[C@@H]3C[C@@H]3C(F)F)ncc12 ; CNc1ncc(C#Cc2cc3ccccn3n2)c2cc(NC(=O)[C@H]3C[C@H]3C(F)F)ncc12]

### Step 1: N¹-methyl-4-(pyrazolo[1,5-a]pyridin-2-ylethynyl)-2,7-naphthyridine-1,6-diamine

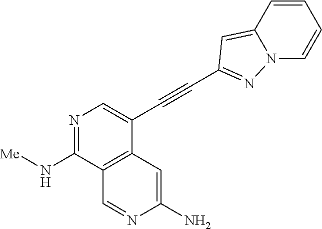 [SMILES: CNc1ncc(C#Cc2cc3ccccn3n2)c2cc(N)ncc12]

To a solution of N-(8-(methylamino)-5-(pyrazolo[1,5-a]pyridin-2-ylethynyl)-2,7-naphthyridin-3-yl)cyclopropanecarboxamide (272 mg; 0.711 mmol; 1.00 eq.) in a mixture solvent of MeOH/DMSO (4:1, 45 mL) was added a solution of NaOH (285 mg; 7.126 mmol; 10.02 eq.) in H₂O (12 mL) dropwise at 0° C. The resulting mixture was stirred at 60° C. for 16 hours under nitrogen atmosphere. The desired product was detected via LCMS. The resulting mixture was concentrated under reduced pressure. The residue was purified by flash chromatography on silica gel column using 1%-9% of MeOH in CH₂Cl₂ as eluent to provide N¹-methyl-4-(pyrazolo[1,5-a]pyridin-2-ylethynyl)-2,7-naphthyridine-1,6-diamine as a yellow crude solid (180 mg, 81.4%). LCMS (ESI) m/z 315.1, [M+H]⁺.

### Step 2: (1R,2S)-2-(difluoromethyl)-N-(8-(methylamino)-5-(pyrazolo[1,5-a]pyridin-2-ylethynyl)-2,7-naphthyridin-3-yl)cyclopropane-1-carboxamide and (1S,2R)-2-(difluoromethyl)-N-(8-(methylamino)-5-(pyrazolo[1,5-a]pyridin-2-ylethynyl)-2,7-naphthyridin-3-yl)cyclopropane-1-carboxamide

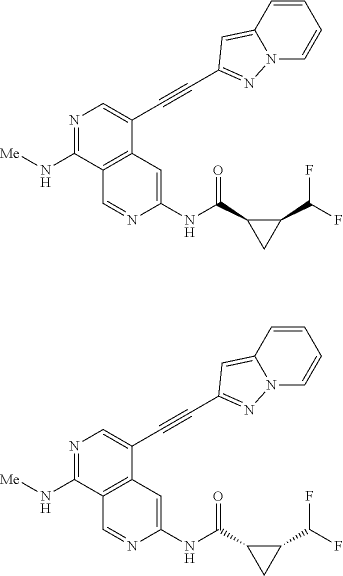 [SMILES: CNc1ncc(C#Cc2cc3ccccn3n2)c2cc(NC(=O)[C@@H]3C[C@@H]3C(F)F)ncc12 ; CNc1ncc(C#Cc2cc3ccccn3n2)c2cc(NC(=O)[C@H]3C[C@H]3C(F)F)ncc12]

To a stirred solution of N¹-methyl-4-(pyrazolo[1,5-a]pyridin-2-ylethynyl)-2,7-naphthyridine-1,6-diamine (140 mg; 0.445 mmol; 1.00 eq.) and 2-(difluoromethyl)cyclopropane-1-carboxylic acid (cis racemate) (55 mg; 0.404 mmol; 0.91 eq.) in pyridine (5 mL) was added POCl₃ (205 mg; 1.337 mmol; 3.00 eq.) dropwise at 0° C. under nitrogen atmosphere. The resulting mixture was stirred at room temperature for 1 hour. The desired product was detected via LCMS. The solvent was concentrated under vacuum. The residue was purified by flash chromatography on silica gel column using 1%-9% of MeOH in CH₂Cl₂ as eluent to provide the cis racemate. The cis racemate was separated by Chiral-HPLC (Column: CHIRALPAK IG, 2×25 cm, 5 μm; Mobile Phase A: Hex (0.5% 2 M NH₃-MeOH), Mobile Phase B: EtOH:DCM=1:1; Flow rate: 20 mL/min; Gradient: 60% B to 60% B in 1.3 min; Wave Length: 220/254 nm; R_{T1}(min): 8.1; R_{T2}(min): 11.49) to provide (1R,2S)-2-(difluoromethyl)-N-(8-(methylamino)-5-(pyrazolo[1,5-a]pyridin-2-ylethynyl)-2,7-naphthyridin-3-yl)cyclopropane-1-carboxamide (Example 754, the faster peak) as a yellow solid (31.8 mg, 16.5%) and (1S′,2R)-2-(difluoromethyl)-N-(8-(methylamino)-5-(pyrazolo[1,5-a]pyridin-2-ylethynyl)-2,7-naphthyridin-3-yl)cyclopropane-1-carboxamide (Example 755, the slower peak) as a yellow solid (30.4 mg, 15.7%). The two configurations are arbitrarily assigned. LCMS (ESI) m/z 433.2, [M+H]⁺.

HNMR for Example 754: ¹H NMR (400 MHz, DMSO-d₆) δ 11.34 (s, 1H), 9.41 (s, 1H), 8.71-8.66 (m, 1H), 8.57 (s, 1H), 8.50-8.41 (m, 1H), 8.37 (s, 1H), 7.75-7.70 (m, 1H), 7.34-7.25 (m, 1H), 7.03-6.95 (m, 1H), 6.89 (s, 1H), 6.21-5.89 (m, 1H), 3.04 (d, J=4.4 Hz, 3H), 2.48-2.38 (m, 1H), 1.98-1.86 (m, 1H), 1.39-1.27 (m, 2H).

HNMR for Example 755: ¹H NMR (400 MHz, DMSO-d₆) δ 11.34 (s, 1H), 9.41 (s, 1H), 8.71-8.66 (m, 1H), 8.57 (s, 1H), 8.50-8.41 (m, 1H), 8.37 (s, 1H), 7.75-7.70 (m, 1H), 7.34-7.25 (m, 1H), 7.03-6.95 (m, 1H), 6.89 (s, 1H), 6.21-5.89 (m, 1H), 3.04 (d, J=4.4 Hz, 3H), 2.48-2.38 (m, 1H), 1.98-1.86 (m, 1H), 1.39-1.25 (m, 2H).

### Examples 756 and 757: Synthesis of (1R,2S)-2-(difluoromethyl)-N-(5-((4-(2-hydroxy-2-methylpropoxy)phenyl)ethynyl)-8-(methylamino)-2,7-naphthyridin-3-yl)cyclopropane-1-carboxamide (Example 756) and (1S,2R)-2-(difluoromethyl)-N-(5-((4-(2-hydroxy-2-methylpropoxy)phenyl)ethynyl)-8-(methylamino)-2,7-naphthyridin-3-yl)cyclopropane-1-carboxamide (Example 757)

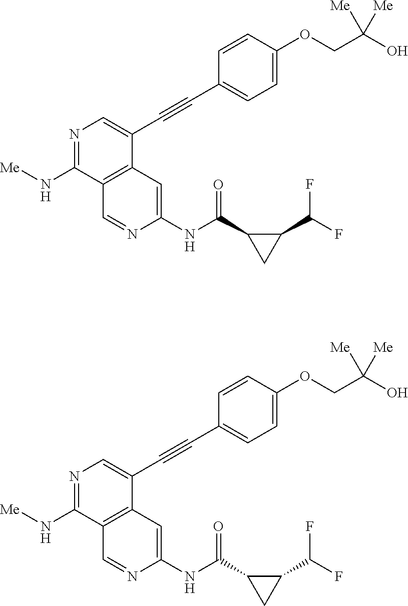 [SMILES: CNc1ncc(C#Cc2ccc(OCC(C)(C)O)cc2)c2cc(NC(=O)[C@@H]3C[C@@H]3C(F)F)ncc12 ; CNc1ncc(C#Cc2ccc(OCC(C)(C)O)cc2)c2cc(NC(=O)[C@H]3C[C@H]3C(F)F)ncc12]

### Step 1: 1-(4-((6-amino-1-(methylamino)-2,7-naphthyridin-4-yl)ethynyl)phenoxy)-2-methylpropan-2-ol

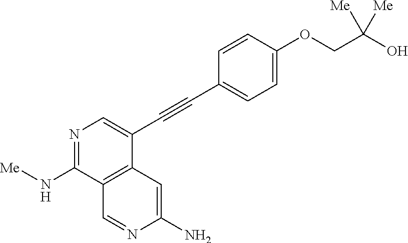 [SMILES: CNc1ncc(C#Cc2ccc(OCC(C)(C)O)cc2)c2cc(N)ncc12]

To a stirred solution of N-(5-((4-(2-hydroxy-2-methylpropoxy)phenyl)ethynyl)-8-(methylamino)-2,7-naphthyridin-3-yl)cyclopropanecarboxamide (Example 714) (250 mg; 0.581 mmol; 1.00 eq.) in a mixture solvent of MeOH/DMSO (5:1, 12 mL) was added a solution of NaOH (232 mg; 5.800 mmol; 9.99 eq.) in H₂O (3.3 mL) dropwise at 0° C. The resulting mixture was stirred at 60° C. for 16 hours under nitrogen atmosphere. The desired product was detected via LCMS. The resulting mixture was concentrated under reduced pressure. The residue was purified by flash chromatography on pre-packed C18 column using 30-70% of MeCN in water (10 mmol/L NH₄HCO₃) to provide 1-(4-((6-amino-1-(methylamino)-2,7-naphthyridin-4-yl)ethynyl)phenoxy)-2-methylpropan-2-ol as a yellow solid (210 mg, 99.7%). LCMS (ESI) m/z 363.2, [M+H]⁺.

### Step 2: (1R,2S)-2-(difluoromethyl)-N-(5-((4-(2-hydroxy-2-methylpropoxy)phenyl)ethynyl)-8-(methylamino)-2,7-naphthyridin-3-yl)cyclopropane-1-carboxamide and (1S,2R)-2-(difluoromethyl)-N-(5-((4-(2-hydroxy-2-methylpropoxy)phenyl)ethynyl)-8-(methylamino)-2,7-naphthyridin-3-yl)cyclopropane-1-carboxamide

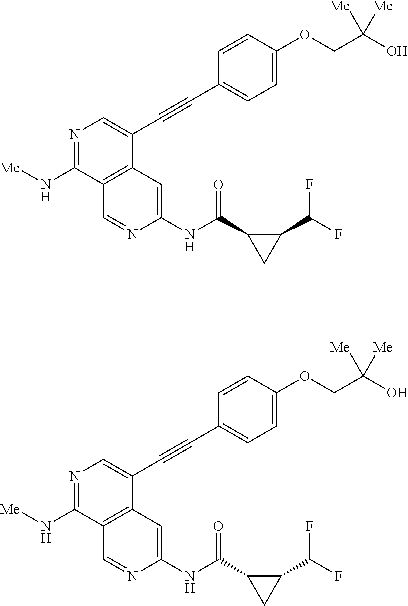 [SMILES: CNc1ncc(C#Cc2ccc(OCC(C)(C)O)cc2)c2cc(NC(=O)[C@@H]3C[C@@H]3C(F)F)ncc12 ; CNc1ncc(C#Cc2ccc(OCC(C)(C)O)cc2)c2cc(NC(=O)[C@H]3C[C@H]3C(F)F)ncc12]

To a stirred solution of 2-(difluoromethyl)cyclopropane-1-carboxylic acid (cis racemate) (34 mg; 0.250 mmol; 0.91 eq.) and 1-(4-((6-amino-1-(methylamino)-2,7-naphthyridin-4-yl)ethynyl)phenoxy)-2-methylpropan-2-ol (100 mg; 0.276 mmol; 1.00 eq.) in pyridine (5 mL) was added POCl₃ (1.27 mg; 0.828 mmol; 3.00 eq.) dropwise at 0° C. under nitrogen atmosphere. The resulting mixture was stirred at room temperature for 0.5 hour. The desired product was detected via LCMS. The resulting mixture was diluted with EtOAc (60 mL) and washed with brine (3×5 mL). The organic layer was dried over anhydrous Na₂SO₄. After filtration, the filtrate was concentrated under reduced pressure. The residue was purified by flash chromatography on silica gel column using 2-10% of MeOH in CH₂Cl₂ as eluent to afford the cis racemate. The cis racemate was separated by Chiral-HPLC (Column: Chiral ART Cellulose-SA, 2×25 cm, 5 μm; Mobile Phase A: Hex (0.5% 2 M NH₃-MeOH), Mobile Phase B: EPA:DCM=1:1; Flow rate: 20 mL/min; Gradient: 40% B to 40% B in 26 min; Wave Length: 220/254 nm; R_{T1}(min): 20.08; R_{T2}(min): 22.73) to provide (1R,2S)-2-(difluoromethyl)-N-(5-((4-(2-hydroxy-2-methylpropoxy)phenyl)ethynyl)-8-(methylamino)-2,7-naphthyridin-3-yl)cyclopropane-1-carboxamide (Example 756, the faster peak) as a yellow solid (21.7 mg, 16.4%) and (1S,2R)-2-(difluoromethyl)-N-(5-((4-(2-hydroxy-2-methylpropoxy)phenyl)ethynyl)-8-(methylamino)-2,7-naphthyridin-3-yl)cyclopropane-1-carboxamide (Example 757, the slower peak) as a yellow solid (22.3 mg, 1.6.8%). The two configurations are arbitrarily assigned. LCMS (ESI) m/z 481.2, [M+H]⁺.

HNMR for example 756: ¹HNMR (400 MHz, DMSO-d₆) δ 11.34 (s, 1H), 9.40 (s, 1H), 8.64 (s, 1H), 8.45-8.32 (m, 1H), 8.26 (s, 1H), 7.55-7.47 (m, 2H), 7.06-6.98 (m, 2H), 6.24-5.92 (m, 1H), 4.66 (s, 1H), 3.77 (s, 2H), 3.03 (d, J=4.4 Hz, 3H), 2.48-2.38 (m, 1H), 1.97-1.89 (m, 1H), 1.41-1.29 (m, 2H), 1.22 (s, 6H).

HNMR for example 757: ¹H NMR (400 MHz, DMSO-d₆) δ 11.34 (s, 1H), 9.40 (s, 1H), 8.64 (s, 1H), 8.45-8.32 (m, 1H), 8.26 (s, 1H), 7.55-7.47 (m, 2H), 7.06-6.98 (m, 2H), 6.24-5.92 (m, 1H), 4.66 (s, 1H), 3.77 (s, 2H), 3.03 (d, J=4.4 Hz, 3H), 2.48-2.38 (m, 1H), 1.97-1.89 (m, 1H), 1.41-1.29 (m, 2H), 1.22 (s, 6H).

### Example 758: Synthesis of N-(5-((3,4-dihydro-1H-benzo[4,5]imidazo[2,1-c][1,4]oxazin-7-yl)ethynyl)-8-(methylamino)-2,7-naphthyridin-3-yl)-1-fluorocyclopropane-1-carboxamide

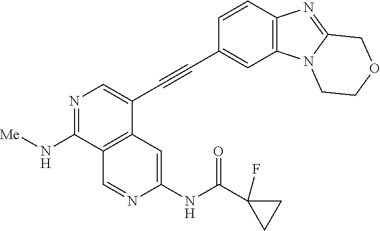 [SMILES: CNc1ncc(C#Cc2ccc3nc4n(c3c2)CCOC4)c2cc(NC(=O)C3(F)CC3)ncc12]

### Step 1: 4-((3,4-dihydro-1H-benzo[4,5]imidazo[2,1-c][1,4]oxazin-7-yl)ethynyl)-N¹-methyl-2,7-naphthyridine-1,6-diamine

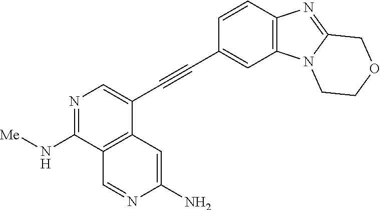 [SMILES: CNc1ncc(C#Cc2ccc3nc4n(c3c2)CCOC4)c2cc(N)ncc12]

To a solution of N-(5-((3,4-dihydro-1H-benzo[4,5]imidazo[2,1-c][1,4]oxazin-7-yl)ethynyl)-8-(methylamino)-2,7-naphthyridin-3-yl)cyclopropanecarboxamide (Example 720) (154.0 mg; 0.351 mmol; 1.00 eq.) in a mixture solvent of MeOH/DMSO (5:1, 6 mL) was added a solution of NaOH (140.4 mg; 3.510 mmol; 1.0.00 eq.) in water (1.5 mL). The resulting solution was stirred at 60° C. for 14 hours. The desired product was detected via LCMS. The solvent was concentrated under vacuum. The residue was purified by flash chromatography on pre-packed C18 column using 50-80% of MeOH in water (10 mmol/L NH₄HCO₃) as eluent to provide 4-((3,4-dihydro-1H-benzo[4,5]imidazo[2,1-c][1,4]oxazin-7-yl)ethynyl)-N¹-methyl-2,7-naphthyridine-1,6-diamine as a yellow solid (54.2 mg, 34%). LCMS (ESI) m/z 371.2, [M+H]⁺.

### Step 2: N-(5-((3,4-dihydro-1H-benzo[4,5]imidazo[2,1-c][1,4]oxazin-7-yl)ethynyl)-8-(methylamino)-2,7-naphthyridin-3-yl)-1-fluorocyclopropane-1-carboxamide

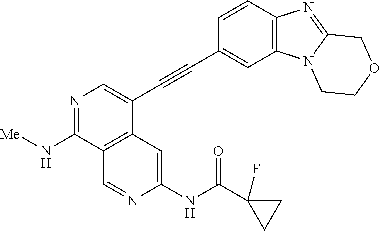 [SMILES: CNc1ncc(C#Cc2ccc3nc4n(c3c2)CCOC4)c2cc(NC(=O)C3(F)CC3)ncc12]

To a solution of 4-((3,4-dihydro-1H-benzo[4,5]imidazo[2,1-c][1,4]oxazin-7-yl)ethynyl)-N¹-methyl-2,7-naphthyridine-1,6-diamine (45.2 mg; 0.122 mmol; 1.00 eq.) and 1-fluorocyclopropane-1-carboxylic acid (11.4 mg; 0.110 mmol; 0.90 eq.) in pyridine (2 mL) was added POCl₃ (56.1 mg; 0.366 mmol; 3.00 eq.) dropwise at 0° C. Then the mixture was stirred at room temperature for 0.5 hour. The reaction was quenched by the addition of water (1 mL) at 0° C. The solvent was concentrated under vacuum. The residue was purified by flash chromatography on silica gel column using 0-20% of MeOH in CH₂Cl₂ as eluent to provide the crude product. The crude product was purified by flash chromatography on pre-packed C18 column using 20-60% of MeOH in water (10 mmol/L NH₄HCO₃) to provide N-(5-((3,4-dihydro-1H-benzo[4,5]imidazo[2,1-c][1,4]oxazin-7-yl)ethynyl)-8-(methylamino)-2,7-naphthyridin-3-yl)-1-fluorocyclopropane-1-carboxamide as a yellow solid (11.1 mg, 19.2%). LCMS (ESI) m/z 457.2, [M+H]⁺. ¹H NMR (400 MHz, DMSO-d₆) δ 10.50 (s, 1H), 9.42 (s, 1H), 8.73 (s, 1H), 8.41 (s, 1H), 8.32 (s, 1H), 7.84 (d, J=1.5 Hz, 1H), 7.64 (d, J=8.3 Hz, 1H), 7.42 (dd, J=8.3, 1.5 Hz, 1H), 4.98 (s, 2H), 4.28-4.15 (m, 4H), 3.04 (s, 3H), 1.52-1.41 (m, 4H).

### Example 759: Synthesis of 1-fluoro-N-(5-((1-methyl-1H-benzo[d][1,2,3]triazol-6-yl)ethynyl)-8-(methylamino)-2,7-naphthyridin-3-yl)cyclopropane-1-carboxamide

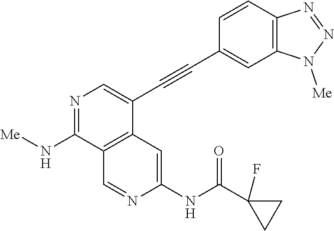 [SMILES: CNc1ncc(C#Cc2ccc3nnn(C)c3c2)c2cc(NC(=O)C3(F)CC3)ncc12]

### Step 1: N¹-methyl-4-((1-methyl-1H-benzo[d][1,2,3]triazol-6-yl)ethynyl)-2,7-naphthyridine-1,6-diamine

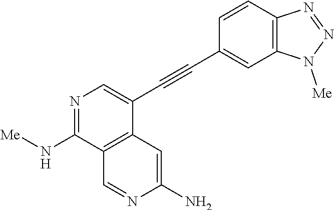 [SMILES: CNc1ncc(C#Cc2ccc3nnn(C)c3c2)c2cc(N)ncc12]

To a stirred solution of N-(5-((1-methyl-1H-benzo[d][1,2,3]triazol-6-yl)ethynyl)-8-(methylamino)-2,7-naphthyridin-3-yl)cyclopropanecarboxamide (Example 595) (300.0 mg; 0.755 mmol; 1.00 eq.) in a mixture solvent of MeOH/DMSO (4:1, 10 mL) was added a solution of NaOH (301.9 mg; 7.550 mmol; 10.00 eq.) in water (2 mL). The resulting mixture was stirred at 60° C. for 16 hours. The desired product was detected via LCMS. The mixture was allowed to cool down to room temperature. The resulting mixture was concentrated under reduced pressure. The residue was purified by flash chromatography on pre-packed C18 column using 20-60% of MeOH in water (10 mmol/L NH₄HCO₃) to provide N¹-methyl-4-((1-methyl-1H-benzo[d][1,2,3]triazol-6-yl)ethynyl)-2,7-naphthyridine-1,6-diamine (150.0 mg, 60.1%) as a light yellow solid. LCMS (ESI) m/z 330.1, [M+H]⁺.

### Step 2: 1-fluoro N-(5-((1-methyl-1H-benzo[d][1,2,3]triazol-6-yl)ethynyl)-8-(methylamino)-2,7-naphthyridin-3-yl)cyclopropane-1-carboxamide

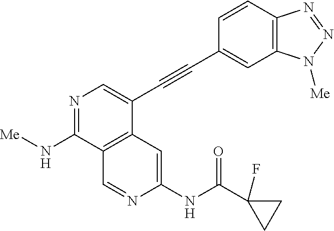 [SMILES: CNc1ncc(C#Cc2ccc3nnn(C)c3c2)c2cc(NC(=O)C3(F)CC3)ncc12]

To a stirred solution of N¹-methyl-4-((1-methyl-1H-benzo[d][1,2,3]triazol-6-yl)ethynyl)-2,7-naphthyridine-1,6-diamine (70.0 mg; 0.213 mmol; 1.00 eq.) and 1-fluorocyclopropane-1-carboxylic acid (1.9.9 mg; 0.192 mmol; 0.90 eq.) in pyridine (2 mL) was added POCl₃ (97.7 mg; 0.639 mmol; 3.00 eq.) dropwise at 0° C. The resulting solution was stirred at room temperature for 0.5 hour. The desired product was detected via LCMS. The resulting mixture was diluted with EtOAc (10 mL) and washed with water (3×10 mL). The organic layer was dried over anhydrous Na₂SO₄. After filtration, the filtrate was concentrated under reduced pressure. The residue was purified by flash chromatography on silica gel column using 5-8% of MeOH in CH₂Cl₂ as eluent to afford 1-fluoro-N-(5-((1-methyl-1H-benzo[d][1,2,3]triazol-6-yl)ethynyl)-8-(methylamino)-2,7-naphthyridin-3-yl)cyclopropane-1-carboxamide as a light yellow solid (26.5 mg, 30.2%). LCMS (ESI) m/z 416.2, [M+H]⁺. ¹H NMR (400 MHz, DMSO-d₆) δ 10.54 (s, 1H), 9.45 (s, 1H), 8.75 (s, 1H), 8.52-8.44 (m, 1H), 8.38 (s, 1H), 8.16 (s, 1H), 8.10 (d, J=8.8 Hz, 1H), 7.55 (dd, J=8.8, 1.2 Hz, 1H), 4.34 (s, 3H), 3.06 (d, J=4.4 Hz, 3H), 1.56-1.41 (m, 4H).

### Example 760: Synthesis of N-(5-((4-((1-acetylazetidin-3-yl)oxy)phenyl)ethynyl)-8-(methylamino)-2,7-naphthyridin-3-yl)-1-fluorocyclopropane-1-carboxamide

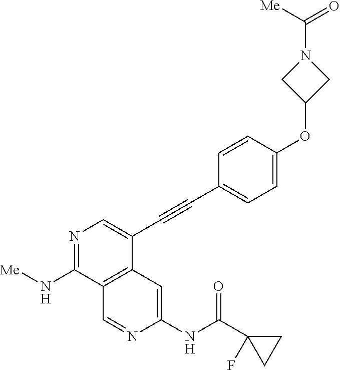 [SMILES: CNc1ncc(C#Cc2ccc(OC3CN(C(C)=O)C3)cc2)c2cc(NC(=O)C3(F)CC3)ncc12]

### Step 1: tert-butyl 3-(4-((6-(cyclopropanecarboxamido)-1-(methylamino)-2,7-naphthyridin-4-yl)ethynyl)phenoxy)azetidine-1-carboxylate

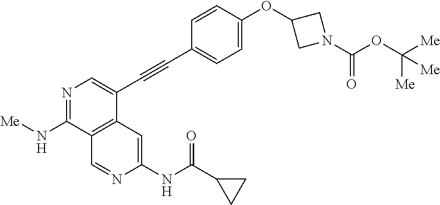 [SMILES: CNc1ncc(C#Cc2ccc(OC3CN(C(=O)OC(C)(C)C)C3)cc2)c2cc(NC(=O)C3CC3)ncc12]

To a solution of N-(5-ethynyl-8-(methylamino)-2,7-naphthyridin-3-yl)cyclopropanecarboxamide (400.0 mg; 1.502 mmol; 1.50 eq.) and tert-butyl 3-(4-bromophenoxy)azetidine-1-carboxylate (Example 715, step 1) (328.6 mg; 1.001 mmol; 1.00 eq.) in DIM (10 mL) were added XPhos (95.4 mg; 0.200 mmol; 0.20 eq.), XPhos Pd G₃ (169.5 mg; 0.200 mmol; 0.20 eq.), CuI (38.1 mg; 0.200 mmol; 0.20 eq.) and Et₃N (405.3 mg; 4.005 mmol; 4.00 eq.) under nitrogen atmosphere. The resulting solution was stirred at 90° C. for 1 hour. The desired product was detected via LCMS. The resulting mixture was diluted with EtOAc (200 mL) and washed with brine (3×20 mL). The organic layer was dried over anhydrous Na₂SO₄. After filtration, the filtrate was concentrated under reduced pressure. The residue was purified by flash chromatography on silica gel column using 2-10% of MeOH in CH₂Cl₂ as eluent to provide tert-butyl 3-(4-((6-(cyclopropanecarboxamido)-1-(methylamino)-2,7-naphthyridin-4-yl)ethynyl)phenoxy)azetidine-1-carboxylate (407.0 mg, 79.1%). LCMS (ESI) m/z 514.2, [M+H]⁺.

### Step 2: tert-butyl 3-(4-((6-amino-1-(methylamino)-2,7-naphthyridin-4-yl)ethynyl)phenoxy)azetidine-1-carboxylate

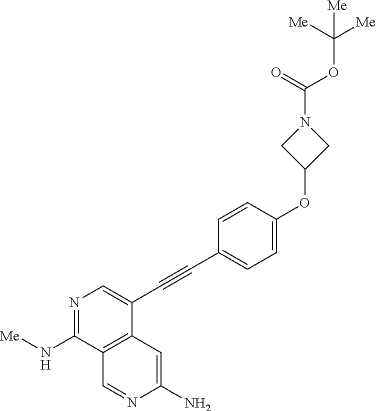 [SMILES: CNc1ncc(C#Cc2ccc(OC3CN(C(=O)OC(C)(C)C)C3)cc2)c2cc(N)ncc12]

To a solution of tert-butyl 3-(4-((6-(cyclopropanecarboxamido)-1-(methylamino)-2,7-naphthyridin-4-yl)ethynyl)phenoxy)azetidine-1-carboxylate (400.0 mg; 0.779 mmol; 1.00 eq.) in a mixture solvent of DMSO/MeOH/THF (1:5:5, 22 mL) was added a solution of NaOH (311.5 mg; 7.790 mmol; 10 eq.) in water. (3 mL). The resulting solution was stirred at 60° C. for 1.6 hours under nitrogen atmosphere. The desired product was detected via LCMS. The resulting mixture was concentrated under reduced pressure. The crude product was purified by flash chromatography on pre-packed C18 column using 50-1.00% of MeOH in water (10 mmol/L NH₄HCO₃) to provide tert-butyl 3-(4-((6-amino-1-(methylamino)-2,7-naphthyridin-4-yl) ethynyl) phenoxy) azetidine-1-carboxylate as a yellow solid (320 mg, 92.4%). LCMS (ESI) m/z 446.2, [M+H]⁺.

### Step 3: 4-((4-(azetidin-3-yloxy)phenyl)ethynyl)-M-methyl-2,7-naphthyridine-1,6-diamine

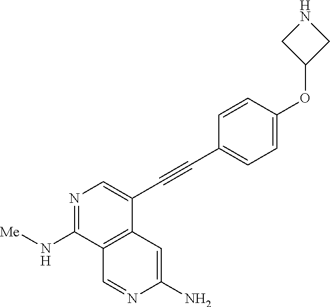 [SMILES: CNc1ncc(C#Cc2ccc(OC3CNC3)cc2)c2cc(N)ncc12]

A solution of tort-butyl 3-(4-((6-amino-1-(methylamino)-2,7-naphthyridin-4-yl)ethynyl)phenoxy)azetidine-1-carboxylate (390.0 mg; 0.904 mmol; 1.00 eq.) in a mixture solvent of trifluoroacetic acid/CH₂Cl₂ (1:5, 6 mL) was stirred at room temperature for 1 hour. The desired product was detected via LCMS. The resulting mixture was concentrated under reduced pressure to afford 4-((4-(azetidin-3-yloxy)phenyl)ethynyl)-N¹-methyl-2,7-naphthyridine-1,6-diamine as yellow solid (480.0 mg, crude). LCMS (ESI) m/z 346.2, [M+H]⁺.

### Step 4: 1-(3-(4-((6-amino-1-(methylamino)-2,7-naphthyridin-4-yl)ethynyl)phenoxy)azetidin-1-yl)ethan-1-one

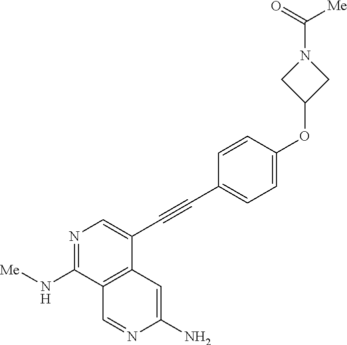 [SMILES: CNc1ncc(C#Cc2ccc(OC3CN(C(C)=O)C3)cc2)c2cc(N)ncc12]

A solution of 4-((4-(azetidin-3-yloxy)phenyl)ethynyl) N¹-methyl-2,7-naphthyridine-1,6-diamine (264.0 mg; 0.764 mmol; 1.00 eq.), DIEA (296.3 mg; 2.292 mmol; 3.00 eq.) and acetic anhydride (78.0 mg; 0.764 mmol; 1.00 eq.) in DMF (3 mL) was stirred at room temperature for 1 hour. The desired product was detected via LCMS. The resulting mixture was purified by flash chromatography on pre-packed C18 column using 30-100% of MeOH in water (10 mmol/L NH₄HCO₃) to provide 1-(3-(4-((6-amino-1-(methylamino)-2,7-naphthyridin-4-yl)ethynyl)phenoxy)azetidin-1-yl)ethan-1-one as yellow solid (147.0 mg, 49.6%). LCMS (ESI) m/z 388.2, [M+H]⁺.

### Step 5: N-(5-((4-((1-acetylazetidin-3-yl)oxy)phenyl)ethynyl)-8-(methylamino)-2,7-naphthyridin-3-yl)-1-fluorocyclopropane-1-carboxamide

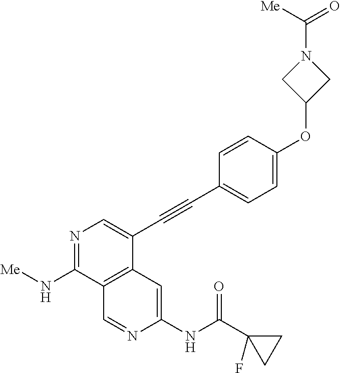 [SMILES: CNc1ncc(C#Cc2ccc(OC3CN(C(C)=O)C3)cc2)c2cc(NC(=O)C3(F)CC3)ncc12]

To a solution of 1-fluorocyclopropane-1-carboxylic acid (14.5 mg; 0.140 mmol; 0.90 eq.) and 1-(3-(4-((6-amino-1-(methylamino)-2,7-naphthyridin-4-yl)ethynyl)phenoxy)azetidin-1-yl)ethan-1-one (60.0 mg; 0.155 mmol; 1.00 eq.) in pyridine (2 mL) was added POCl₃ (71.2 mg; 0.465 mmol; 3.00 eq.) dropwise at 0 GC. The reaction was stirred at room temperature for 30 minutes. The solvent was concentrated under vacuum. The residue was purified by flash chromatography on silica gel column using 2-10% of MeOH in CH₂C1, as eluent to provide the crude product. The crude product was purified by flash chromatography on pre-packed C18 column using 30-100% of MeOH in water (10 mmol/L NH₄HCO₃) to provide N-(5-((4-((1-acetylazetidin-3-yl)oxy)phenyl)ethynyl)-8-(methylamino)-2,7-naphthyridin-3-yl)-1-fluorocyclopropane-1-carboxamide as a yellow solid (46.1 mg, 62.8%). LCMS (ESI) m/z 474.2, [M+H]⁺. ¹H NMR (400 MHz, DMSO-d₆) δ 10.44 (s, 1H), 9.42 (s, 1H), 8.63 (s, 1H), 8.41-8.33 (m, 1H), 8.30 (s, 1H), 7.56-7.48 (d, J=8.8 Hz, 2H), 6.97-6.89 (d, J=8.8 Hz, 2H), 5.13-5.03 (m, 1H), 4.62-4.53 (m, 1H), 4.35-4.26 (m, 1H), 4.15-4.07 (m, 1H), 3.83-3.75 (m, 1H), 3.04 (d, J=4.4 Hz, 3H), 1.81 (s, 3H), 1.53-1.38 (m, 4H).

### Example 761: Synthesis of N-(5-((1-ethyl-2-methyl-1H-benzo[d]imidazol-6-yl)ethynyl)-8-(methylamino)-2,7-naphthyridin-3-yl)cyclopropanecarboxamide

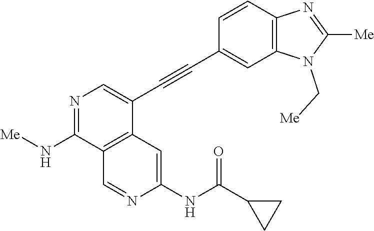 [SMILES: CCn1c(C)nc2ccc(C#Cc3cnc(NC)c4cnc(NC(=O)C5CC5)cc34)cc21]

### Step 1: 6-bronco-1-ethyl-2-methyl-1H-benzo[d]imidazole and 5-bromo-1-ethyl-2-methyl-1H-benzo[d]imidazole

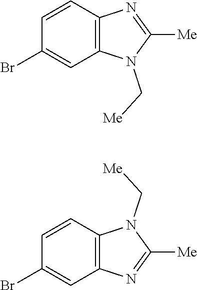 [SMILES: CCn1c(C)nc2ccc(Br)cc21 ; CCn1c(C)nc2cc(Br)ccc21]

To a solution of 5-bromo-2-methyl-3H-1,3-benzodiazole (1.00 g; 4.738 mmol; 1.00 eq.) and Cs₂CO₃ (4.63 g; 14.214 mmol; 3.00 eq) in DMF (20 mL) was added ethyl iodide (738.9 mg; 4.738 mmol; 1.00 eq.) at 0° C. The mixture was stirred at room temperature for 2 hours. The desired product was detected via LCMS. After filtration, the filter cake was washed with EtOAc (3×30 mL), the combined filtrate was washed with brine (4×20 mL). The organic layer was dried over anhydrous Na₂SO₄. After filtration, the filtrate was concentrated under reduced pressure. The residue was purified by Prep-Achiral-SFC (Column: DAICEL DCpak P4VP 3×25 cm, 5 μm; Mobile Phase A: CO₂, Mobile Phase B: MeOH (0.1% 2 M NH₃-MeOH); Flow rate: 60 mL/min; Gradient: isocratic 17% B; Column Temperature(° C.): 35; Back Pressure(bar): 100; Wave Length: 254 nm; R_{T1}(min): 3.28; R_{T2}(min): 3.81) to provide 6-bromo-1-ethyl-2-methyl-1H-benzo[d]imidazole (the faster peak) as a white solid (353.7 mg, 31.2%) and 5-bromo-1-ethyl-2-methyl-1H-benzo[d]imidazole (the slower peak) as a white solid (282.2 mg, 24.9%). LCMS (ESI) m/z 239.0, [M+H]⁺.

### Step 2: N-(5-((1-ethyl-2-methyl-1H-benzo[d]imidazol-6-yl)ethynyl)-8-(methylamino)-2,7-naphthyridin-3-yl)cyclopropanecarboxamide

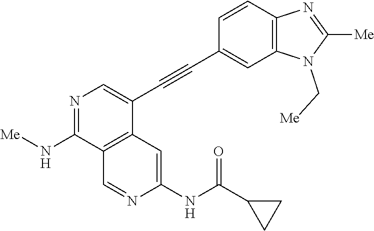 [SMILES: CCn1c(C)nc2ccc(C#Cc3cnc(NC)c4cnc(NC(=O)C5CC5)cc34)cc21]

A solution of N-(5-ethynyl-8-(methylamino)-2,7-naphthyridin-3-yl)cyclopropanecarboxamide (80.0 mg; 0.300 mmol; 1.00 eq.), 6-bromo-1-ethyl-2-methyl-1H-benzo[d]imidazole (71.8 mg; 0.300 mmol; 1.00 eq.), XPhos Pd G₃ (25.4 mg; 0.030 mmol; 0.10 eq.), XPhos (14.3 mg; 0.030 mmol; 0.10 eq.), Cut (5.7 mg; 0.030 mmol; 0.10 eq.) and triethylamine (121.6 mg; 1.202 mmol; 4.00 eq.) in DMF (4 mL) was stirred at 90° C. for 1 hour under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to afford N-(5-((1-ethyl-2-methyl-1H-benzo[d]imidazol-6-yl)ethynyl)-8-(methylamino)-2,7-naphthyridin-3-yl)cyclopropanecarboxamide as a yellow solid (51.1 mg, 40.0%). LCMS (ESI) m/z 425.2, [M+H]⁺. ¹H NMR (400 MHz, DMSO-d₆) δ 11.10 (s, 1H), 9.38 (s, 1H), 8.81 (s, 1H), 8.40-8.24 (m, 2H), 7.85 (d, J=1.2 Hz, 1H), 7.56 (d, J=8.0 Hz, 1H), 7.33 (dd, J=8.0, 1.2 Hz, 1H), 4.40-4.00 (m, 2H), 3.03 (d, J=4.4 Hz, 3H), 2.57 (s, 3H), 2.17-2.08 (m, 1H), 1.35 (t, J=7.2 Hz, 3H), 0.97-0.75 (m, 4H).

### Example 762: Synthesis of N-(5-((1-ethyl-2-methyl-1H-benzo[d]imidazol-5-yl)ethynyl)-8-(methylamino)-2,7-naphthyridin-3-yl)cyclopropanecarboxamide

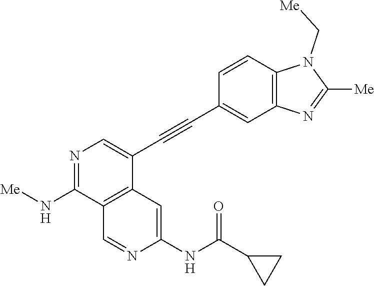 [SMILES: CCn1c(C)nc2cc(C#Cc3cnc(NC)c4cnc(NC(=O)C5CC5)cc34)ccc21]

A solution of N-(5-ethynyl-8-(methylamino)-2,7-naphthyridin-3-yl)cyclopropanecarboxamide (70.0 mg; 0.263 mmol; 1.00 eq.), 5-bromo-1-ethyl-2-methyl-1H-benzo[d]imidazole (Example 761, step 1) (62.9 mg; 0.263 mmol; 1.00 eq.), XPhos Pd G₃ (22.3 mg; 0.026 mmol; 0.10 eq.), XPhos (12.5 mg; 0.026 mmol; 0.10 eq.), CuI (5.0 mg; 0.026 mmol; 0.10 eq.) and triethylamine (106.4 mg; 1.052 mmol; 4.00 eq.) in DMF (4.0 mL) was stirred at 90° C. for 1 hour under nitrogen atmosphere. The desired product was detected via LCMS. The resulting mixture was concentrated under vacuum. The residue was purified by flash chromatography on silica gel column using 0-10% of MeOH in CH₂Cl₂ as eluent to provide N-(5-((1-ethyl-2-methyl-1H-benzo[d]imidazo-5-yl)ethynyl)-8-(methylamino)-2,7-naphthyridin-3-yl)cyclopropanecarboxamide as a yellow solid (49.8 mg, 44.4%). LCMS (ESI) m/z 425.2, [M+H]⁺. ¹H NMR (400 MHz, DMSO-d₆) δ 11.07 (s, 1H), 9.38 (s, 1H), 8.71 (s, 1H), 8.30 (s, 1H), 8.28-8.1.7 (m, 1H), 7.70 (d, J=1.6 Hz, 1H), 7.59 (d, J=8.0 Hz, 1H), 7.40 (dd, J=8.0, 1.6 Hz, 1H), 4.32-4.19 (m, 2H), 3.03 (d, J=4.4 Hz, 3H), 2.57 (s, 3H), 2.14-2.06 (m, 1H), 1.32 (t, J=7.2 Hz, 3H), 0.94-0.89 (m, 2H), 0.87-0.81 (m, 2H).

### Example 763: Synthesis of N-(5-((1-(difluoromethyl)-2-methyl-1H-benzo[d]imidazol-6-yl)ethynyl)-8-(methylamino)-2,7-naphthyridin-3-yl)cyclopropanecarboxamide

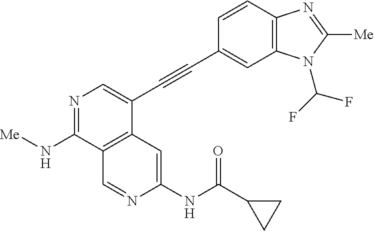 [SMILES: CNc1ncc(C#Cc2ccc3nc(C)n(C(F)F)c3c2)c2cc(NC(=O)C3CC3)ncc12]

### Step 1: 6-bromo-1-(difluoromethyl)-2-methyl-1H-benzo[d]imidazole and 5-bromo-1-(difluoromethyl)-2-methyl-1H-benzo[d]imidazole

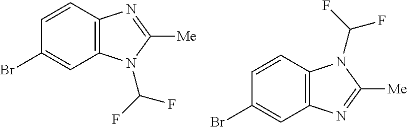 [SMILES: Cc1nc2ccc(Br)cc2n1C(F)F ; Cc1nc2cc(Br)ccc2n1C(F)F]

A solution of 6-bromo-2-methyl-1H-benzo[d]imidazole (500.0 mg; 2.369 mmol; 1.00 eq.), diethyl (bromodifluoromethyl)phosphonate (1.26 g; 4.738 mmol; 2.00 eq.) and KF (275.2 mg, 4.738 mmol; 2.00 eq.) in acetonitrile (5 mL) was stirred at 35° C. overnight under nitrogen atmosphere. The resulting mixture was concentrated under reduced pressure. The residue was purified by flash chromatography on silica gel column using 10-50% of EtOAc in petroleum ether as eluent to provide 6-bromo-1-(difluoromethyl)-2-methyl-1H-benzo[d]imidazole as an off-white solid (110.0 mg, 17.7%) and 5-bromo-1-(difluoromethyl)-2-methyl-1H benzo[d]imidazole as an off-white solid (200.0 mg, 32.3%). LCMS (ESI) m/z 261.0, [M+H]⁺.

### Step 2: N-(5-((1-(difluoromethyl)-2-methyl-1H-benzo[d]imidazol-6-yl)ethynyl)-8-(methylamino)-2,7-naphthyridin-3-yl)cyclopropanecarboxamide

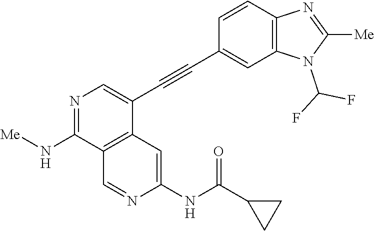 [SMILES: CNc1ncc(C#Cc2ccc3nc(C)n(C(F)F)c3c2)c2cc(NC(=O)C3CC3)ncc12]

A solution of 6-bromo-1-(difluoromethyl)-2-methyl-1H-benzo[d]imidazole (65.3 mg; 0.251 mmol; 1.00 eq.), XPhos Pd G₃ (42.4 mg; 0.050 mmol; 0.20 eq.), XPhos (23.9 mg; 0.050 mmol; 0.20 eq.), Et₃N (76.0 mg; 0.752 mmol; 3.00 eq.), CuI (9.5 mg; 0.050 mmol; 0.20 eq.) and N-(5-ethynyl-8-(methylamino)-2,7-naphthyridin-3-yl)cyclopropanecarboxamide (100.0 mg; 0.376 mmol; 1.50 eq.) in DMF (4 mL) was stirred at 90° C. for 1 hour under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide N-(5-((1-(difluoromethyl)-2-methyl-1H-benzo[d]imidazol-6-yl)ethynyl)-8-(methylamino)-2,7-naphthyridin-3-yl)cyclopropanecarboxamide as a yellow solid (45.7 mg, 40.8%). LCMS (ESI) m/z 447.2, [M+H]⁺. ¹H NMR (400 MHz, DMSO-d₆) δ 11.11 (s, 1H), 9.38 (s, 1H), 8.77 (s, 1H), 8.48-8.25 (m, 2H), 8.17-7.95 (m, 2H), 7.69 (d, J=8.4 Hz, 1H), 7.48 (d, J=8.4 Hz, 1H), 3.03 (d, J=4.4 Hz, 3H), 2.70 (s, 3H), 2.18-2.06 (m, 1H), 1.00-0.78 (m, 4H).

### Example 764: Synthesis of A-(5-((1-(difluoromethyl)-2-methyl-1H-benzo[d]imidazol-5-yl)ethynyl)-8-(methylamino)-2,7-naphthyridin-3-yl)cyclopropanecarboxamide

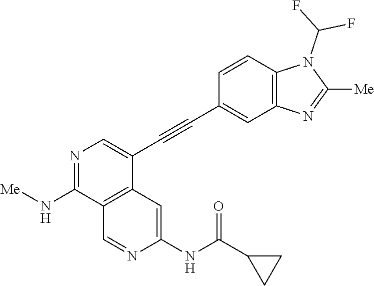 [SMILES: CNc1ncc(C#Cc2ccc3c(c2)nc(C)n3C(F)F)c2cc(NC(=O)C3CC3)ncc12]

A solution of 5-bromo-1-(difluoromethyl)-2-methyl-1H-benzo[d]imidazole (Example 763, step 1) (80.0 mg; 0.306 mmol; 1.00 eq.), XPhos (29.2 mg; 0.061 mmol; 0.20 eq.), XPhos Pd G₃ (51.9 mg; 0.061 mmol; 0.20 eq.), CuI (11.8 mg; 0.061 mmol; 0.20 eq.), triethylamine (93.0 mg; 0.918 mmol; 3.00 eq.) and N-(5-ethynyl-8-(methylamino)-2,7-naphthyridin-3-yl)cyclopropanecarboxamide (122.4 mg; 0.459 mmol; 1.50 eq.) in DMF (4 mL) was stirred at 90° C. for 1 hour under nitrogen atmosphere. The desired product was detected via LCMS. The resulting mixture was concentrated under reduced pressure. The residue was purified by flash chromatography on silica gel column using 0-1.0% of MeOH in CH₂Cl₂ as eluent to provide N-(5-((1-(difluoromethyl)-2-methyl-1H-benzo[d₁]imidazol-5-yl)ethynyl)-8-(methylamino)-2,7-naphthyridin-3-yl)cyclopropanecarboxamide as a yellow solid (71.3 mg, 51.5%). LCMS (ESI) m/z 447.2, [M+H]⁺. ¹H NMR (400 MHz, DMSO-d₆) δ 11.08 (s, 1H), 9.38 (s, 1H), 8.71 (s, 1H), 8.37-8.28 (m, 2H), 8.11 (t, J=57.6 Hz, 1H), 7.81 (d, J=1.6 Hz, 1H), 7.74 (d, J=8.0 Hz, 1H), 7.52 (dd, J=8.0, 1.6 Hz, 1H), 3.03 (d, J=4.4 Hz, 3H), 2.70 (s, 3H), 2.16-2.02 (m, 1H), 0.98-0.78 (m, 4H).

### Example 765: Synthesis of 1V-(5-(6-methoxypyrazolo[1,5-a]pyridin-2-yl)-8-(methylamino)-2,7-naphthyridin-3-yl)cyclopropanecarboxamide

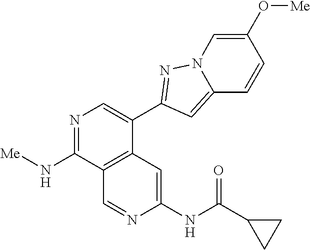 [SMILES: CNc1ncc(-c2cc3ccc(OC)cn3n2)c2cc(NC(=O)C3CC3)ncc12]

### Step 1: ethyl 2-(5-methoxypyridin-2-yl)acetate

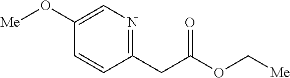 [SMILES: CCOC(=O)Cc1ccc(OC)cn1]

To a solution of 2-(5-methoxypyridin-2-yl)acetonitrile (500.3 mg; 3.345 mmol; 1.00 eq.) in EtOH (5 mL) was added HCl (gas) (4 M 1,4-dioxane, 5 mL). The resulting solution was stirred at 60° C. for 1 hour under nitrogen atmosphere. The desired product was detected via LCMS. The mixture was allowed to cool down to room temperature. The resulting mixture was concentrated under reduced pressure. The residue was purified by flash chromatography on pre-packed C18 column using 20-50% of MeOH in water (10 mmol/L NH₄HCO₃) to provide ethyl 2-(5-methoxypyridin-2-yl)acetate as a yellow oil (385.9 mg, 58.6%). LCMS (ESI) m/z 196.1, [M+H]⁺.

### Step 2: 6-methoxypyrazolo[1,5-a]pyridin-2-ol

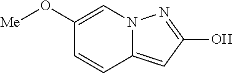 [SMILES: COc1ccc2cc(O)nn2c1]

A solution of ethyl 2-(5-methoxypyridin-2-yl)acetate (300.2 mg; 1.514 mmol; 1.00 eq.) and O-(mesitylsulfonyl)hydroxylamine (1.22 g; 5.584 mmol; 3.70 eq.) in CH₂Cl₂ (20 mL) was stirred at room temperature overnight under nitrogen atmosphere. The desired product was detected via LCMS. The resulting mixture was concentrated under reduced pressure. The residue was purified by flash chromatography on silica gel column using 5-8% of MeOH in CH₂Cl₂ as eluent to provide 6-methoxypyrazolo[1,5-a]pyridin-2-ol (180.1 mg, 59.5%) as a yellow solid. LCMS (ESI) m/z 165.1, [M+H]⁺.

### Step 3: 6-methoxypyrazolo[1,5-a]pyridin-2-yl trifluoromethanesulfonate

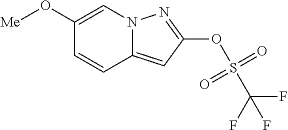 [SMILES: COc1ccc2cc(OS(=O)(=O)C(F)(F)F)nn2c1]

To a stirred solution of 6-methoxypyrazolo[1,5-a]pyridin-2-ol (180.1 mg; 1.084 mmol; 1.00 eq.) in a mixture solvent of THF/DMF (1:1, 2 mL) was added NaH (60%) (108.4 mg; 2.710 mmol; 2.50 eq.) at 0° C. under nitrogen atmosphere. After stirring for 10 minutes to the above solution was added 1,1,1-trifluoro-N-phenyl-N trifluoromethanesulfonylmethanesulfonamide (468.1 mg; 1.293 mmol; 1.20 eq.) at 0° C. The resulting mixture was stirred at room temperature for 1 hour. The desired product was detected via LCMS. The reaction was quenched with water (10 mL) and extracted with EtOAc (3×20 mL). The combined organic layers were washed with brine (3×20 mL) and dried over anhydrous Na₂SO₄. After filtration, the filtrate was concentrated under reduced pressure. The residue was purified by flash chromatography on silica gel column using 20-30% of EtOAc in petroleum ether as eluent to provide 6-methoxypyrazolo[1,5-a]pyridin-2-yl trifluoromethanesulfonate as a white solid (240.3 mg, 73.8%). LCMS (ESI) m/z 297.0, [M+H]⁺.

### Step 4: N-(5-(6-methoxypyrazolo[1,5-a]pyridin-2-yl)-8-(methylamino)-2,7-naphthyridin-3-yl)cyclopropanecarboxamide

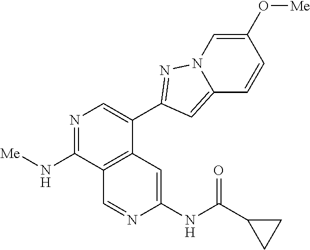 [SMILES: CNc1ncc(-c2cc3ccc(OC)cn3n2)c2cc(NC(=O)C3CC3)ncc12]

A solution of N-[8-(methylamino)-5-(4,4,5,5-tetramethyl-1,3,2-dioxaborolan-2-yl)-2,7-naphthyridin-3-yl]cyclopropanecarboxamide (80.2 mg; 0.211 mmol; 1.00 eq.) and 6-methoxypyrazolo[1,5-a]pyridin-2-yl trifluoromethanesulfonate (149.2 mg; 0.501 mmol; 2.50 eq.) in a mixture solvent of 1,4-dioxane/water. (5:1, 1 mL). To the above solution were added XPhos Pd G₃ (22.4 mg; 0.02 mmol; 0.01 eq.), XPhos (25.6 mg; 0.052 mmol; 0.02 eq.) and K₃PO₄ (172.2 mg; 0.813 mmol; 4.00 eq.) under nitrogen atmosphere. The resulting mixture was stirred at 90° C. for 1 hour. The desired product was detected via LCMS. The mixture was allowed to cool down to room temperature. Then was concentrated under reduced pressure. The residue was purified by flash chromatography on silica gel column using 5-8% of MeOH in CH₂Cl₂ as eluent to provide the crude product. The crude product was purified by flash chromatography on pre-packed C18 column using 50-70% of MeCN in water (10 mmol/L NH₄HCO₃) to provide N-(5-(6-methoxypyrazolo[1,5-a]pyridin-2-yl)-8-(methylamino)-2,7-naphthyridin-3-yl)cyclopropanecarboxamide as a yellow solid (18.5 mg, 21.9%). LCMS (ESI) m/z 389.2, [M+H]⁺. ¹H NMR (400 MHz, DMSO-d₆) δ 10.97 (s, 1H), 9.40 (s, 1H), 8.87 (s, 1H), 8.44 (d, 0.1=2.2 Hz, 1H), 8.27 (s, 1H), 8.21-8.02 (m, 1H), 7.66 (d, J=9.6 Hz, 1H), 7.10-7.04 (m, 1H), 6.75 (s, 1H), 3.85 (s, 3H), 3.04 (d, J=4.4 Hz, 3H), 2.07-2.02 (m, 1H), 0.95-0.81 (m, 4H).

### Example 766: Synthesis of N-(5-((6-methoxypyrazolo[1,5-a]pyridin-2-yl)ethynyl)-8-(methylamino)-2,7-naphthyridin-3-yl)cyclopropanecarboxamide

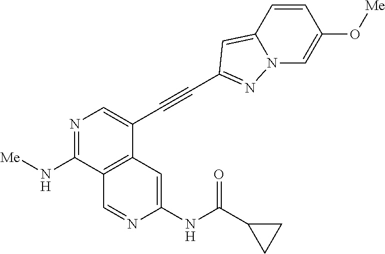 [SMILES: CNc1ncc(C#Cc2cc3ccc(OC)cn3n2)c2cc(NC(=O)C3CC3)ncc12]

### Step 1: 6-methoxypyrazolo[1,5-a]pyridin-2-yl trifluoromethanesulfonate

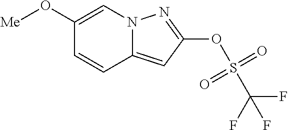 [SMILES: COc1ccc2cc(OS(=O)(=O)C(F)(F)F)nn2c1]

To a stirred solution of 6-methoxypyrazolo[1,5-a]pyridin-2-ol (Example 765, step 2) (180.1 mg; 1.084 mmol; 1.00 eq.) in a mixture solvent of THF/DMF (1:1, 2 mL) was added NaH (60% dispersion in mineral oil; 108.4 mg; 2.710 mmol; 2.50 eq.) and 1,1,1-trifluoro-N-phenyl-N-trifluoromethanesulfonylmethanesulfonamide (468.1 mg; 1.293 mmol; 1.20 eq.) at 0° C. The resulting mixture was stirred at room temperature for 1 hour under nitrogen atmosphere. The desired product was detected via LCMS. The reaction was quenched with water (20 mL) at 0° C. and extracted with EtOAc (3×20 mL). The combined organic layers were washed with brine (3×10 mL) and dried over anhydrous Na₂SO₄. After filtration, the filtrate was concentrated under reduced pressure. The residue was purified by flash chromatography on silica gel column using 20-30% of EtOAc in petroleum ether as eluent to provide 6-methoxypyrazolo[1,5-a]pyridin-2-yl trifluoromethanesulfonate as a white solid (240.3 mg, 73.8%). LCMS (ESI) m/z 297.0, [M+H]⁺.

### Step 2: N-(5-((6-methoxypyrazolo[1,5-a]pyridin-2-yl)ethynyl)-8-(methylamino)-2,7-naphthyridin-3-yl)cyclopropanecarboxamide

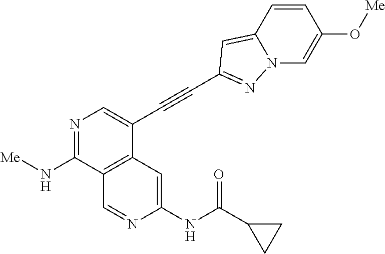 [SMILES: CNc1ncc(C#Cc2cc3ccc(OC)cn3n2)c2cc(NC(=O)C3CC3)ncc12]

To a stirred solution of 6-methoxypyrazolo[1,5-a]pyridin-2-yl trifluoromethanesulfonate (70.3 mg; 0.236 mmol; 1.00 eq.) and N-(5-ethynyl-8-(methylamino)-2,7-naphthyridin-3-yl)cyclopropanecarboxamide (62.9 mg; 0.236 mmol; 1.00 eq.) in DMF (1 mL) were added XPhos Pd G₃ (20.0 mg; 0.024 mmol; 0.10 eq.), XPhos (25.6 mg; 0.052 mmol; 0.20 eq.), Et₃N (74.2 mg; 0.733 mmol; 3.00 eq.) and CuI (4.5 mg; 0.024 mmol; 0.10 eq.). The resulting mixture was stirred at 110° C. for 1 hour under nitrogen atmosphere. The desired product was detected via LCMS. The mixture was allowed to cool down to room temperature and concentrated under reduced pressure. The residue was purified by flash chromatography on silica gel column using 5-8% of MeOH in CH₂Cl₂ as eluent to provide the crude product. Then the crude product was re crystallized with a mixture solvent of CH₂Cl₂/MeOH (1:1, 5 mL) to afford N-(5-((6-methoxypyrazolo[1, 5-a]pyridin-2-yl)ethynyl)-8-(methylamino)-2,7-naphthyridin-3-1.5 yl)cyclopropanecarboxamide as a yellow solid (54.1 mg, 55.5%). LCMS (ESI) m/z 413.2, [M+H]⁺. ¹H NMR (400 MHz, DMSO-d₆) δ 11.08 (s, 1H), 9.39 (s, 1H), 8.60 (s, 1H), 8.41 (d, J=2.4 Hz, 1H), 8.40-8.34 (m, 1H), 8.33 (s, 1H), 7.64 (d, J=9.6 Hz, 1H), 7.10 (dd, J=9.6, 2.4 Hz, 1H), 6.82 (s, 1H), 3.85 (s, 3H), 3.03 (d, J=4.4 Hz, 3H), 2.1.2-2.04 (m, 1H), 0.91-0.79 (m, 4H).

### Example 767: Synthesis of N-(5-((6-fluoropyrazolo[1,5-a]pyridin-2-yl)ethynyl)-8-(methylamino)-2,7-naphthyridin-3-yl)cyclopropanecarboxamide

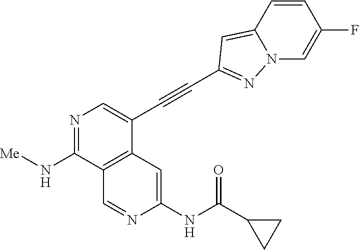 [SMILES: CNc1ncc(C#Cc2cc3ccc(F)cn3n2)c2cc(NC(=O)C3CC3)ncc12]

### Step 1: 6-fluoropyrazolo[1,5-a]pyridin-2-ol

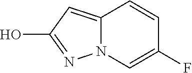 [SMILES: Oc1cc2ccc(F)cn2n1]

A solution of ethyl 2-(5-fluoropyridin-2-yl)acetate (200.3 mg; 1.092 mmol; 1.00 eq.) and O-(mesitylsulfonyl)hydroxylamine (1.00 g; 4.645 mmol; 3.90 eq.) in CH₂Cl₂ (5 mL) was stirred at room temperature overnight under nitrogen atmosphere. The desired product was detected via LCMS. The resulting mixture was concentrated under reduced pressure. The residue was purified by flash chromatography on silica gel column using 5-8% of MeOH in CH₂Cl₂ as eluent to provide 6-fluoropyrazolo[1,5-a]pyridin-2-ol as a red solid (70.2 mg, 42.1%). LCMS (ESI) m/z 153.0, [M+H]⁺.

### Step 2: 6-fluoropyrazolo[1,5-a]pyridin-2-yl trifluoromethanesulfonate

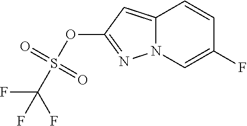 [SMILES: O=S(=O)(Oc1cc2ccc(F)cn2n1)C(F)(F)F]

To a stirred solution of 6-fluoropyrazolo[1,5-a]pyridin-2-ol (60.3 mg; 0.394 mmol; 1.00 eq.) and DIPEA (150.1 mg; 1.161 mmol; 3.00 eq.) in CH₂Cl₂ (2 mL) was added Tf₂O (166.8 mg; 0.591 mmol; 1.50 eq.) in portions at 0° C. The resulting mixture was stirred at room temperature for 2 hours. The desired product was detected via LCMS. The resulting mixture was diluted with CH₂Cl₂ (20 mL), washed with brine (2×5 mL). The organic layer was dried with Na₂SO₄. After filtration, the filtrate was concentrated under reduced pressure to afford 6-fluoropyrazolo[1,5-a]pyridin-2-yl trifluoromethanesulfonate as a colorless oil (100.2 mg, 89.2%). LCMS (ESI) m/z 285.0, [M+H]⁺.

### Step 3: N-(5-((6-fluoropyrazolo[1,5-a]pyridin-2-yl)ethynyl)-8-(methylamino)-2,7-naphthyridin-3-yl)cyclopropanecarboxamide

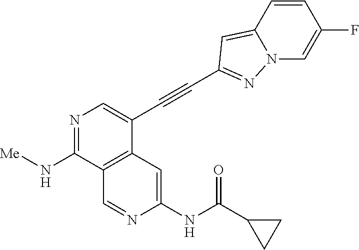 [SMILES: CNc1ncc(C#Cc2cc3ccc(F)cn3n2)c2cc(NC(=O)C3CC3)ncc12]

To a stirred solution of 6-fluoropyrazolo[1,5-a]pyridin-2-yl trifluoromethanesulfonate (70.2 mg; 0.246 mmol; 1.00 eq.) and N-(5-ethynyl-8-(methylamino)-2,7-naphthyridin-3-yl)cyclopropanecarboxamide (65.5 mg; 0.244 mmol; 1.00 eq.) in DMF (1 mL) were added XPhos Pd G₃ (20.3 mg; 0.024 mmol; 0.10 eq.), XPhos (23.7 mg; 0.048 mmol; 0.20 eq.), Et₃N (74.2 mg; 0.733 mmol; 3.00 eq.) and CuI (4.6 mg; 0.024 mmol; 0.1.0 eq.). The resulting mixture was stirred at 110° C. for 1 hour under nitrogen atmosphere. The desired product was detected via LCMS. The mixture was allowed to cool down to room temperature and concentrated under reduced pressure. The residue was purified by flash chromatography on silica gel column using 5-8% of MeOH in CH₂Cl₂ as eluent to provide the crude product. Then the crude product was re crystallized with a mixture solvent of CH₂Cl₂/MeOH (1:1, 5 mL) to afford N-(5-((6-fluoropyrazolo[1,5-a]pyridin-2-yl)ethynyl)-8-(methylamino)-2,7-naphthyridin-3-yl)cyclopropanecarboxamide as a yellow solid (65.2 mg, 66.1%). LCMS (ESI) m/z 401.1, [M+H]⁺. ¹H NMR (400 MHz, DMSO-d₆) δ 11.09 (s, 1H), 9.39 (s, 1H), 9.06-9.00 (m, 1H), 8.60 (s, 1H), 8.43-8.38 (m, 1H), 8.35 (s, 1H), 7.85-7.78 (m, 1H), 7.46-7.39 (m, 1H), 6.95 (s, 1H), 3.04 (d, J=4.4 Hz, 3H), 2.13-2.04 (m, 1H), 0.92-0.80 (m, 4H).

### Example 768: Synthesis of (S)—N-(5-((4-((1,4-dioxan-2-yl)methoxy)phenyl)ethynyl)-8-(methylamino)-2,7-naphthyridin-3-yl)cyclopropanecarbaxamide

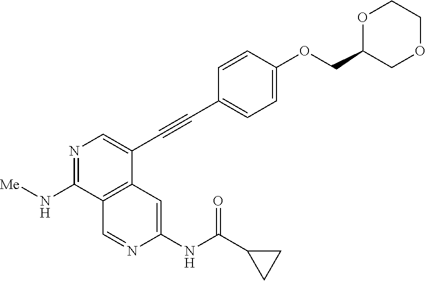 [SMILES: CNc1ncc(C#Cc2ccc(OC[C@@H]3COCCO3)cc2)c2cc(NC(=O)C3CC3)ncc12]

### Step 1: (S)-2-((4-bromophenoxy)methyl)-1,4-dioxane

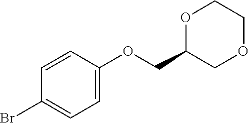 [SMILES: Brc1ccc(OC[C@@H]2COCCO2)cc1]

To a solution of 4-bromophenol (200.0 mg; 1.156 mmol; 1.00 eq.), (R)-(1,4-dioxan-2-yl)methanol (110.0 mg; 0.931 mmol; 0.81 eq.) and PPh₃ (335.1 mg; 1.278 mmol; 1.11 eq.) in THF (5 mL) was added DIAD (235.0 mg; 1.162 mmol; 1.01 eq.) at 0° C. The resulting solution was stirred at room temperature for 3 hours under nitrogen atmosphere. The desired product was detected via LCMS. The resulting mixture was concentrated under vacuum. The residue was purified by flash chromatography on silica gel column using 5-60% of EtOH in petroleum ether as eluent to provide (S)-2-((4-bromophenoxy)methyl)-1,4-dioxane as a white oil (135.0 mg, 42.7%). LCMS (ESI) m/z 273.0, [M+H]⁺.

### Step 2: (S)—N-(5-((4-((1,4-dioxan-2-yl)methoxy)phenyl)ethynyl)-8-(methylamino)-2,7-naphthyridin-3-yl)cyclopropanecarboxamide

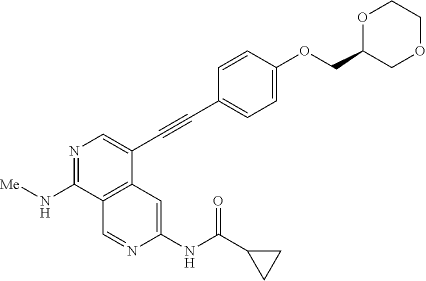 [SMILES: CNc1ncc(C#Cc2ccc(OC[C@@H]3COCCO3)cc2)c2cc(NC(=O)C3CC3)ncc12]

A solution of N-(5-ethynyl-8-(methylamino)-2,7-naphthyridin-3-yl)cyclopropanecarboxamide (150.0 mg; 0.563 mmol; 1.00 eq.), XPhos Pd G₃ (63.5 mg; 0.075 mmol; 0.13 eq.), XPhos (35.7 mg; 0.075 mmol; 0.13 eq.), Et₃N (151.8 mg; 1.500 mmol; 2.66 eq.), CuI (14.3 mg; 0.075 mmol; 0.13 eq.) and (5)-2-((4-bromophenoxy)methyl)-1,4-dioxane (102.0 mg; 0.373 mmol; 0.66 eq.) in DMF (4 mL) was stirred at 110° C. for 1 hour under nitrogen atmosphere. The desired product was detected via LCMS. The resulting mixture was diluted with EtOAc (50 mL) and washed with brine (3×5 mL). The organic layer was dried over anhydrous Na₂SO₄. After filtration, the filtrate was concentrated under reduced pressure. The residue was purified by flash chromatography on silica gel column using 0-10% of MeOH in CH₂Cl₂ as eluent to provide the crude product. The crude product was purified by flash chromatography on pre-packed C18 column using 20-100% of MeOH in water (10 mmol/L NH₄HCO₃) as eluent to provide (S)—N-(5-((4-((1,4-dioxan-2-yl)methoxy)phenyl)ethynyl)-8-(methylamino)-2,7-naphthyridin-3-yl)cyclopropanecarboxamide as a yellow solid (88.9 mg, 34.4%). LCMS (ESI) m/z 459.2, [M+H]⁺. ¹H NMR (400 MHz, DMSO-d₆) δ 11.07 (s, 1H), 9.37 (s, 1H), 8.64 (s, 1H), 8.32-8.22 (m, 2H), 7.49 (d, J=8.4 Hz, 2H), 7.02 (d, J=8.4 Hz, 2H), 4.02 (d, J=4.8 Hz, 2H), 3.93-3.74 (m, 3H), 3.74-3.59 (m, 2H), 3.59-3.44 (m, 1H), 3.44-3.37 (m, 1H), 3.01 (d, J=4.4 Hz, 3H), 2.18-1.98 (m, 1H), 0.94-0.75 (m, 4H).

### Example 769: Synthesis of N-(5-((4-(dimethylphosphoryl)phenyl)ethynyl)-8-(methylamino)-2,7-naphthyridin-3-yl)cyclopropanecarboxamide

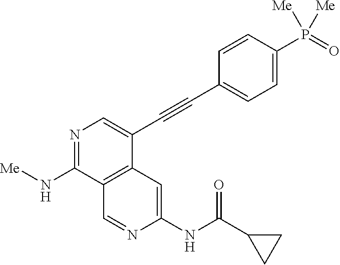 [SMILES: CNc1ncc(C#Cc2ccc(P(C)(C)=O)cc2)c2cc(NC(=O)C3CC3)ncc12]

### Step 1: (1-bromophenyl)dimethylphosphine oxide

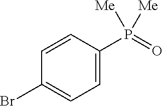 [SMILES: CP(C)(=O)c1ccc(Br)cc1]

To a stirred solution of 1-bromo-4-iodobenzene (1.01 g; 3.535 mmol; 1.00 eq.) and hydroxydimethylphosphane (278.2 mg; 3.562 mmol; 1.00 eq.) in 1,4-dioxane (10 mL) were added Pd₂(dba)₃ (324.0 mg; 0.354 mmol; 0.10 eq. XantPhos (409.5 mg; 0,707 mmol; 0.20 eq.) and Et₃N (419.3 mg; 4.141 mmol; 1.18 eq.). The mixture was stirred at 100° C. overnight under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide (4-bromophenyl)dimethylphosphine oxide as a white solid (600.1 mg, 72.8%). LCMS (ESI) m/z 233.0, [M+H]⁺.

### Step 2: N-(5-04-(dimethylphosphoryl)phenyl)ethynyl)-8-(methylamino)-2,7-naphthyridin-3-yl)cyclopropanecarboxamide

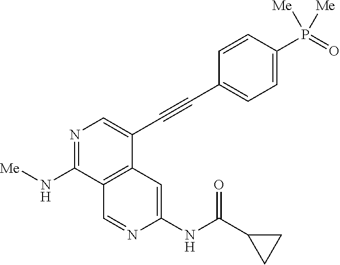 [SMILES: CNc1ncc(C#Cc2ccc(P(C)(C)=O)cc2)c2cc(NC(=O)C3CC3)ncc12]

To a stirred solution of (4-bromophenyl)dimethylphosphine oxide (100.3 mg; 0.429 mmol; 1.50 eq.) and N-(5-ethynyl-8-(methylamino)-2,7-naphthyridin-3-yl)cyclopropanecarboxamide (75.4 mg; 0.282 mmol; 1.00 eq.) in DMF (1.5 mL) were added XPhos Pd G₃ (23.8 mg; 0.028 mmol; 0.10 eq.), XPhos (26.8 mg; 0.056 mmol; 0.20 eq.), Et₃N (85.3 mg; 0.840 mmol; 3.00 eq.) and CuI (5.3 mg; 0.028 mmol; 0.10 eq.). The mixture was stirred at 90° C. for 1 hour under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide the crude product. Then the crude product was recrystallized with a mixture solvent of CH₂Cl₂/MeOH (1:1, 5 mL) to afford N-(5-04-(dimethylphosphoryl)phenylethynyl)-8-(methylamino)-2,7-naphthyridin-3-ypcyclopropanecarboxamide as a yellow solid (36.5 mg, 30.9%). LCMS (ESI) m/z 419.2, [ ]N,4:lit ¹H NMR, (400 MHz, DMSO-d₆) δ 11.11 (s, 1H), 9.39 (s, 1H), 8.69 (s, 1H), 8.42-8.37 (m, 1H), 8.32 (s, 1H), 7.89-7.79 (m, 2H), 7.72-7.65 (m, 2H), 3.04 (d, J=4.4 Hz, 31-),2.14-2.05 (m, 1H), 1.70 (s, 3H), 1.67 (s, 3H), 0.95-0.81 (m, 4H).

### Example 770: Synthesis of N-(8-(methylamino)-5-((4-((1-propionylazetidin-3-yl)oxy)phenyl)ethynyl)-2,7-naphthyridin-3-yl) cyclopropanecarboxamide

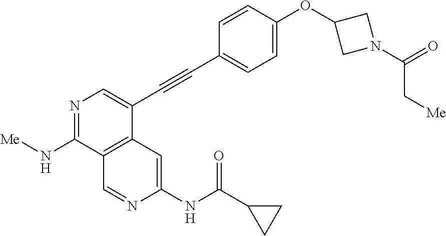 [SMILES: CCC(=O)N1CC(Oc2ccc(C#Cc3cnc(NC)c4cnc(NC(=O)C5CC5)cc34)cc2)C1]

### Step 1: tert-butyl 3-(4-bromophenoxy)azetidine-1-carboxylate

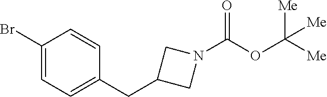 [SMILES: CC(C)(C)OC(=O)N1CC(Cc2ccc(Br)cc2)C1]

To a solution of 4-bromophenol (1.00 g; 5.880 mmol; 1.00 eq.) and Cs₂CO₃ (3.79 g; 11.760 mmol; 2.00 eq.) in DMF (24 mL) was added tert-butyl 3-bromoazetidine-1-carboxylate (2.07 g; 8.808 mmol; 1.50 eq.) under nitrogen atmosphere. The reaction was stirred at 120° C. for 1 hour. The desired product was detected via LCMS. The resulting mixture was diluted with EtOAc (200 mL) and washed with brine (3×20 mL) and dried over anhydrous Na₂SO₄. After filtration, the filtrate was concentrated under reduced pressure. The residue was purified by flash chromatography on silica gel column using 10-50% of EtOAc in petroleum ether as eluent to provide tert-butyl 3-(4-bromophenoxy)azetidine-1-carboxylate as white solid (2.07 g, 97.9%). LCMS (ESI) m/z 328.0, [M+H]⁺.

### Step 2: 3-(4-bromophenoxy)azetidine 2,2,2-trifluoroacetate

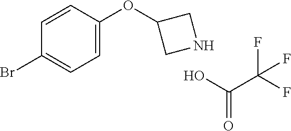

To a solution of tert-butyl 3-(4-bromophenoxy)azetidine-1-carboxylate (100.0 mg; 0.305 mmol; 1.00 eq.) in CH₂Cl₂ (3 mL) was added 2,2,2-trifluoroacetic acid (0.6 mL) under nitrogen atmosphere. The reaction was stirred at room temperature for 1 hour. The desired product was detected via LCMS. The solvent was concentrated under vacuum to afford 3-(4-bromophenoxy)azetidine 2,2,2-trifluoroacetate as a white solid (132.0 mg, crude). LCMS (ESI) m/z 228.0, [M+H]⁺.

### Step 3: 1-(3-(4-bromophenoxy)azetidin-1-yl)propan-1-one

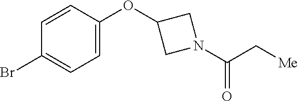 [SMILES: CCC(=O)N1CC(Oc2ccc(Br)cc2)C1]

To a solution of 3-(4-bromophenoxy)azetidine 2,2,2-trifluoroacetate (100.0 mg; 0.307 mmol; 1.00 eq.), DIEA (118.9 mg; 0.921 mmol; 3.00 eq.) in a mixture solvent of CH₂Cl₂/DMF (5:1, 3 mL) was added propionic anhydride (47.9 mg; 0.368 mmol; 1.20 eq.) under nitrogen atmosphere. The reaction was stirred at room temperature for 1 hour. The desired product was detected via LCMS. The solvent was concentrated under vacuum. The residue was purified by flash chromatography on pre-packed C18 column using 20-100% of MeOH in water (10 mmol/L, NH₄HCO₃) as eluent to provide 1-(3-(4-bromophenoxy)azetidin-1-yl)propan-1-one as a white solid (70.0 mg, 80.3%). LCMS (ESI) m/z 284.0, [M+H]⁺.

### Step 4: N-(8-(methylamino)-5-((4-((1-propionylazetidin-3-yl)oxy)phenyl)ethynyl)-2,7-naphthyridin-3-yl) cyclopropanecarboxamide

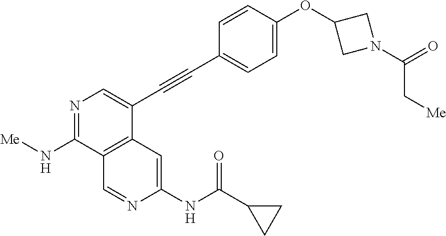 [SMILES: CCC(=O)N1CC(Oc2ccc(C#Cc3cnc(NC)c4cnc(NC(=O)C5CC5)cc34)cc2)C1]

A solution of N-(5-ethynyl-8-(methylamino)-2,7-naphthyridin-3-yl)cyclopropanecarboxamide (70.0 mg; 0.263 mmol; 1.00 eq.), 1-(3-(4-bromophenoxy)azetidin-1-yl)propan-1-one (49.0 mg; 0.172 mmol; 0.66 eq.), XPhos (24.0 mg; 0.050 mmol; 0.19 eq.), XPhos Pd G₃ (43.0 mg; 0.051 mmol; 0.19 eq.), CuI (10.0 mg; 0.053 mmol; 0.20 eq.) and Et₃N (70 mg; 0.692 mmol; 2.63 eq.) in DMF (4 mL) was stirred at 11.0° C. for 1 hour under nitrogen atmosphere. The desired product was detected via LCMS. The solvent was concentrated under vacuum. The residue was purified by flash chromatography on silica gel column using 2-10% of MeOH in CH₂Cl₂ as eluent to provide N-(8-(methylamino)-5-((4-((1-propionylazetidin-3-yl)oxy)phenyl)ethynyl)-2,7-naphthyridin-3-yl) cyclopropanecarboxamide as a yellow solid (39.5 mg, 32.0%). LCMS (ESI) m/z 470.2, [M+H]⁺. ¹H NMR (400 MHz, DMSO-d₆) δ 11.06 (s, 1H), 9.37 (s, 1H), 8.64 (s, 1H), 8.31-8.26 (m, 1H), 8.25 (s, 1H), 7.51 (d, J=8.8 Hz, 2H), 6.93 (d, J=8.8 Hz, 2H), 5.23-4.95 (m, 1H), 4.68-4.53 (m, 1H), 4.36-4.21 (m, 1H), 4.17-4.06 (m, 1H), 3.84-3.70 (m, 1H), 3.02 (d, J=4.4 Hz, 3H), 2.19-1.99 (m, 3H), 0.98 (t, J=7.2 Hz, 3H), 0.93-0.80 (m, 4H).

### Example 771: Synthesis of N-(5-((4-((1-(cyclopropanecarbonyl)azetidin-3-yl)oxy)phenyl)ethynyl)-8-(methylamino)-2,7-naphthyridin-3-yl)cyclopropanecarboxamide

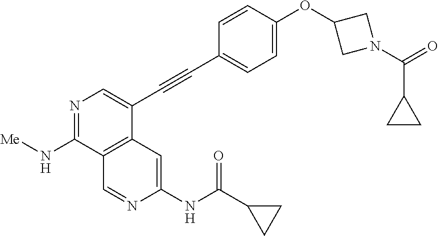 [SMILES: CNc1ncc(C#Cc2ccc(OC3CN(C(=O)C4CC4)C3)cc2)c2cc(NC(=O)C3CC3)ncc12]

### Step 1: 3-(4-bromophenoxy)azetidine hydrochloride

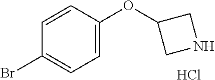 [SMILES: Brc1ccc(OC2CNC2)cc1 ; Cl]

To a solution of tert-butyl 3-(4-bromophenoxy)azetidine-1-carboxylate (Example 770, step 1) (300.0 mg; 0.914 mmol; 1.00 eq.) in MeOH (3 mL) was added HCl (gas) (4 M in 1,4-dioxane, 1.5 mL) at 0° C. The reaction mixture was stirred at room temperature for 2 hours. The desired product was detected via LCMS. The resulting mixture was concentrated under reduced pressure to afford 3-(4-bromophenoxy)azetidine hydrochloride as a white solid (244.0 mg, crude). LCMS (ESI) m/z 228.0, [M+H]⁺.

### Step 2: (3-(4-bromophenoxy)azetidin-1-yl)(cyclopropyl)methanone

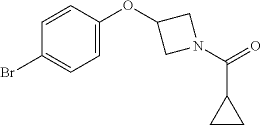 [SMILES: O=C(C1CC1)N1CC(Oc2ccc(Br)cc2)C1]

To a solution of 3-(4-bromophenoxy)azetidine hydrochloride (244.0 mg; 0.922 mmol; 1.00 eq.) and DIEA (357.6 mg; 2.766 mmol; 3.00 eq.) in CH₂Cl₂ (3.0 mL) was added cyclopropanecarbonyl chloride (192.8 mg; 1.844 mmol; 2.00 eq.). The mixture was stirred at room temperature for 1 hour. The desired product was detected via LCMS. The resulting mixture was diluted with CH₂Cl₂ (50 mL) and washed with brine (3×5 mL). The organic layer was concentrated under vacuum. The residue was purified by flash chromatography on pre-packed C18 column using 40-70% of MeOH in water (10 mmol/L NH₄HCO₃) as eluent to provide (3-(4-bromophenoxy)azetidin-1-yl)(cyclopropyl)methanone as a yellow solid (232.3 mg, 85.0%). LCMS (ESI) m/z 296.0, [M+H]⁺.

### Step 3: N-(5-((4-((1-(cyclopropanecarbonyl)azetidin-3-yl)oxy)phenyl)ethynyl)-8-(methylamino)-2,7-naphthyridin-3-yl)cyclopropanecarboxamide

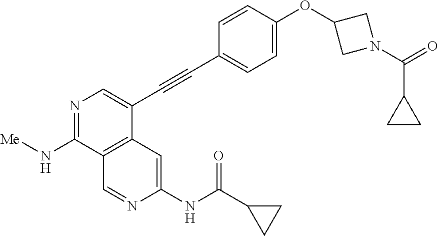 [SMILES: CNc1ncc(C#Cc2ccc(OC3CN(C(=O)C4CC4)C3)cc2)c2cc(NC(=O)C3CC3)ncc12]

A solution of N-(5-ethynyl-8-(methylamino)-2,7-naphthyridin-3-yl)cyclopropanecarboxamide (80.0 mg; 0.300 mmol; 1.00 eq.), (3-(4-bromophenoxy)azetidin-1-yl)(cyclopropyl)methanone (88.9 mg; 0.300 mmol; 1.00 eq.), XPhos Pd G₃ (25.4 mg; 0.030 mmol; 0.10 eq.), XPhos (14.3 mg; 0.030 mmol; 0.10 eq.), CuI (5.7 mg; 0.030 mmol; 0.1.0 eq.) and triethylamine (121.6 mg; 1.200 mmol; 4.00 eq.) in DMF (4.0 mL) was stirred at 90° C. for 1 hour under nitrogen atmosphere. The desired product was detected via LCMS. The resulting mixture was concentrated under vacuum. The residue was purified by flash chromatography on silica gel column using 0-6% of MeOH in CH₂Cl₂ as eluent to afford the crude product. The crude product was purified by flash chromatography on pre-packed C18 column using 30-55% of THE in water (10 mmol/L NH₄HCO₃) as eluent to provide N-(5-((4-((1-(cyclopropanecarbonyl)azetidin-3-yl)oxy)phenyl)ethynyl)-8-(methylamino)-2,7-naphthyridin-3-yl)cyclopropanecarboxamide as a yellow solid (72.4 mg, 49.8%). LCMS (ESI) m/z 482.1, [M+H]⁺. ¹H NMR (400 MHz, DMSO-d₆) δ 11.07 (s, 1H), 9.37 (s, 1H), 8.64 (s, 1H), 8.39-8.28 (m, 1H), 8.25 (s, 1H), 7.54-7.49 (m, 2H), 6.97-6.93 (m, 2H), 5.22-5.05 (m, 1H), 4.80-4.60 (m, 1H), 4.48-4.27 (m, 1H), 4.25-4.05 (m, 1H), 3.90-3.71 (m, 1H), 3.02 (d, J=4.4 Hz, 3H), 2.12-2.04 (m, 1H), 1.60-1.51 (m, 1H), 0.92-0.80 (m, 4H), 0.77-0.69 (m, 4H).

### Example 772: Synthesis of N-(5-((4-(2H-1,2,3-triazol-2-yl)phenyl)ethynyl)-8-(methylamino)-2,7-naphthyridin-3-yl)cyclopropanecarboxamide

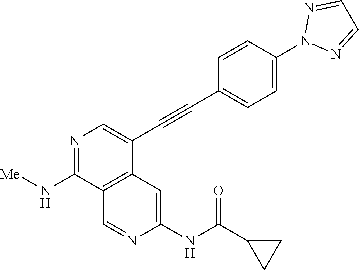 [SMILES: CNc1ncc(C#Cc2ccc(-n3nccn3)cc2)c2cc(NC(=O)C3CC3)ncc12]

A solution of N-(5-ethynyl-8-(methylamino)-2,7-naphthyridin-3-yl)cyclopropanecarboxamide (80.0 mg; 0.300 mmol; 1.00 eq.), 2-(4-bromophenyl)-2H-1,2,3-triazole (67.3 mg; 0,300 mmol; 1.00 eq.), XPhos Pd G₃ (25.4 mg; 0.030 mmol; 0.10 eq.), XPhos (14.3 mg; 0.030 mmol; 0.10 eq.), CuI (5.7 mg; 0.030 mmol; 0.10 eq.) and triethylamine (121.60 mg; 1.200 mmol, 4.00 eq.) in DMF (4.0 mL) was stirred at 90° C. for 1 hour under nitrogen atmosphere. The desired product was detected via LCMS. The resulting mixture was concentrated under vacuum. The residue was purified by flash chromatography on silica gel column using 0-5% of MeOH in CH₂Cl₂ as eluent to provide N-(5-((4-(2H-1,2,3-triazol-2-yl)phenyl)ethynyl)-8-(methylamino)-2,7-naphthyridin-3-yl)cyclopropanecarboxamide as a yellow solid (90.8 mg, 73.7%). LCMS (ESI) m/z 410.2, [M+H]⁺. ¹H NMR, (400 MHz, DMSO-d₆) δ 11.10 (s, 1H), 9.38 (s, 1H), 8.67 (s, 1H), 8.43-8.35 (s, 1H), 8.32 (s, 1H), 8.18 (d, J=6.4 Hz, 2H), 8.15-8.04 (m, 2H), 7.82-7.70 (m, 2H), 3.03 (d, J=4.4 Hz, 3H), 2.14-2.04 (m, 1H), 1.00-0.90 (m, 2H), 0.90-0.78 (m, 2H).

### Example 773: Synthesis of N-(5-((2-(1,1-difluoroethyl)-1-methyl-1H-benzo[d]imidazol-6-yl)ethynyl)-5(methylamino)-2,7-naphthyridin-3-yl)cyclopropanecarboxamide

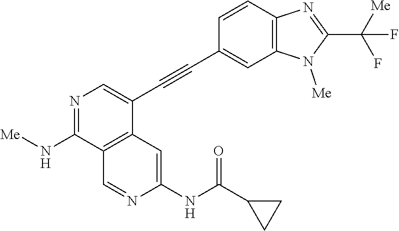 [SMILES: CNc1ncc(C#Cc2ccc3nc(C(C)(F)F)n(C)c3c2)c2cc(NC(=O)C3CC3)ncc12]

### Step 1: 6-bromo-2-(1,1-difluoroethyl)-1-methyl-1H-benzo[d]imidazole

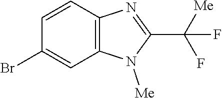 [SMILES: Cn1c(C(C)(F)F)nc2ccc(Br)cc21]

A solution of 5-bromo-N¹-methylbenzene-1,2-diamine (200.2 mg; 0.995 mmol; 1.00 eq.) and 2,2-difluoropropanoic acid (550.1 mg; 4.997 mmol; 5.00 eq.) in HCl (3 M in water, 3 mL) was stirred at 100° C. overnight under nitrogen atmosphere. The desired product was detected via LCMS. The resulting mixture was diluted with water (50 mL), neutralized to pH=7 with saturated Na₂CO₃ solution. The resulting mixture was extracted with EtOAc (3×20 mL). The combined organic layers were washed with brine, dried over anhydrous Na₂SO₄. After filtration, the filtrate was concentrated under reduced pressure. The residue was purified by flash chromatography on silica gel column using 5-10% of EtOAc in petroleum ether as eluent to provide 6-bromo-2-(1,1-difluoroethyl)-1-methyl-1H-benzo[d]imidazole as a yellow solid (200.0 mg, 73.0%). LCMS (ESI) m/z 275.0, [M+H]⁺.

### Step 2: N-(5-((2-(1,1-difluoroethyl)-1-methyl-1H-benzo[d]imidazol-6-yl)ethynyl)-5(methylamine)-2,7-naphthyridin-3-yl)cyclapropanecarboxamide

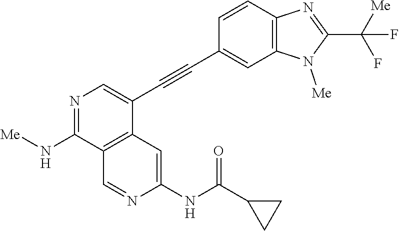 [SMILES: CNc1ncc(C#Cc2ccc3nc(C(C)(F)F)n(C)c3c2)c2cc(NC(=O)C3CC3)ncc12]

To a stirred solution of 6-bromo-2-(1,1-difluoroethyl)-1-methyl-1H benzo[d]imidazole (90.2 mg; 0.327 mmol; 1.50 eq.) and N-(5-ethynyl-8-(methylamino)-2,7-naphthyridin-3-yl)cyclopropanecarboxamide (60.1 mg; 0.225 mmol; 1.00 eq.) in DMF (1.5 mL) were added XPhos Pd G₃ (19.2 mg; 0.023 mmol; 0.10 eq.), XPhos (21.5 mg; 0.045 mmol; 0.20 eq.), Et₃N (70.1 mg; 0.693 mmol; 3.00 eq.) and CuI (4.2 mg; 0.022 mmol; 0.10 eq.). The resulting mixture was stirred at 90° C. for 1 hour under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide the crude product. Then the crude product was re-crystallized with a mixture solvent of CH₂Cl₂/MeOH (1:1, 5 mL) to afford N-(5-((2-(1,1-difluoroethyl)-1-methyl-1H-benzo[d]imidazol-6-yl)ethynyl)-8-(methylamino)-2,7-naphthyridin-3-yl)cyclopropanecarboxamide as a yellow solid (34.2 mg, 32.9%). LCMS (ESI) m/z 461.2, [M+H]⁺. ¹H NMR (400 MHz, DMSO-d₆) δ 11.13 (s, 1H), 9.39 (s, 1H), 8.80 (s, 1H), 8.38-8.31 (m, 1H), 8.30 (s, 1H), 8.05 (d, J=1.2 Hz, 1H), 7.80 (d, J=8.4 Hz, 1H), 7.48 (dd, J=8.4, 1.2 Hz, 1H), 4.02 (s, 3H), 3.03 (d, J=4.4 Hz, 3H), 2.25 (t, J=19.6 Hz, 3H), 2.14-2.06 (m, 1H), 0.92-0.83 (m, 4H).

### Example 774: Synthesis of N-(5-((1-methyl-2-(2,2,2-trifluoroethyl)-1H-benzo[d]imidazol-6-yl)ethynyl)-8-(methylamino)-2,7-naphthyridin-3-yl)cyclopropanecarboxamide

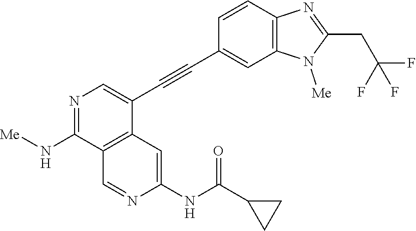 [SMILES: CNc1ncc(C#Cc2ccc3nc(CC(F)(F)F)n(C)c3c2)c2cc(NC(=O)C3CC3)ncc12]

### Step 1: 6-bromo-1-methyl-2-(2,2,2-trifluoroethyl)-.1H-benzo[d]imidazole

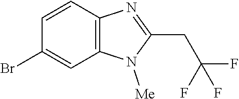 [SMILES: Cn1c(CC(F)(F)F)nc2ccc(Br)cc21]

To a solution of 5-bromo-N¹-methylbenzene-1,2-diamine (400.0 mg; 1.989 mmol; 1.00 eq.) in HCl (3 M in water, 6 mL) was added 3,3,3-trifluoropropanoic acid (1.27 g; 9.945 mmol; 5.00 eq.) at room temperature. The resulting solution was stirred at 100° C. overnight. The desired product was detected via LCMS. The mixture was allowed to cool down to room temperature. The resulting mixture was diluted with water (10 mL) and neutralized to pH=7 with saturated NaHCO₃ solution. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by flash chromatography on silica gel column using 5-8% of MeOH in CH₂Cl₂ as eluent to provide 6-bromo-1-methyl-2-(2,2,2-trifluoroethyl)-1H benzo[d]imidazole as a yellow solid (70.0 mg, 12.0%). LCMS (ESI) m/z 293.0, [M+H]⁺.

### Step 2: N-(5-((1-methyl-2-(2,2,2-trifluoroethyl)-1H-benzo[d]imidazol-6-yl)ethynyl)-8-(methylamino)-2,7-naphthyridin-3-yl)cyclopropanecarboxamide

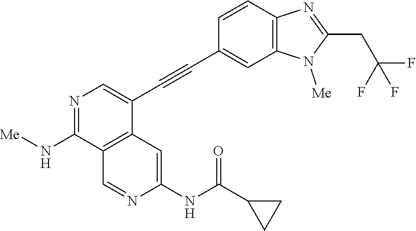 [SMILES: CNc1ncc(C#Cc2ccc3nc(CC(F)(F)F)n(C)c3c2)c2cc(NC(=O)C3CC3)ncc12]

To a stirred solution of 6-bromo-1-methyl-2-(2,2,2-trifluoroethyl)-1H-benzo[d]imidazole (70.0 mg; 0.238 mmol; 1.50 eq.) and N-(5-ethynyl-8-(methylamino)-2,7-naphthyridin-3-yl)cyclopropanecarboxamide (42.4 mg; 0.1.59 mmol; 1.00 eq.) in DMF (1 mL) were added XPhos Pd G₃ (13.5 mg; 0.016 mmol; 0.10 eq.), XPhos (15.2 mg; 0.032 mmol; 0.20 eq.), Et₃N (80.6 mg; 0.795 mmol; 5.00 eq.), CuI (3.0 mg; 0.016 mmol; 0.10 eq.). The resulting mixture was stirred at 110° C. overnight under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH: in CH₂Cl₂ as eluent to provide N-(5-((1-methyl-2-(2,2,2-trifluoroethyl)-1H-benzo[d]imidazol-6-yl)ethynyl)-8-(methylamino)-2,7-naphthyridin-3-yl)cyclopropanecarboxamide as a light yellow solid (28.2 mg, 37.0%). LCMS (ESI) m/z 479.2, [M+H]⁺. ¹H NMR (400 MHz, DMSO-d₆) δ 11.12 (s, 1H), 9.39 (s, 1H), 8.80 (s, 1H), 8.34-8.30 (m, 1H), 8.28 (s, 1H), 7.94 (d, J=1.2 Hz, 1H), 7.68 (d, J=8.4 Hz, 1H), 7.40 (dd, J=8.4, 1.2 Hz, 1H), 4.30-4.16 (m, 2H), 3.88 (s, 3H), 3.03 (d, J=4.4 Hz, 3H), 2.15-2.06 (m, 1H), 0.93-0.83 (m, 4H).

### Example 775: Synthesis of (1S,2R)—N-(5-((2-(difluoromethyl)-1-methyl-1H-benzo[d]imidazol-6-yl)ethynyl)-8-(methylamino)-2,7-naphthyridin-3-yl)-2-methylcyclopropane-1-carboxamide

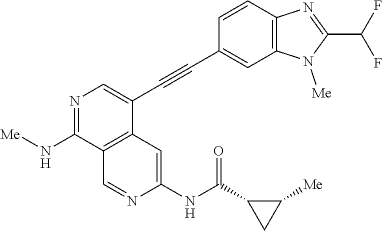 [SMILES: CNc1ncc(C#Cc2ccc3nc(C(F)F)n(C)c3c2)c2cc(NC(=O)[C@H]3C[C@H]3C)ncc12]

### Step 3: 4-((2-(difluoromethyl)-1-methyl-1H-benzo[d]imidazol-6-yl)ethynyl)-N¹-methyl-2,7-naphthyridine-1,6-diamine

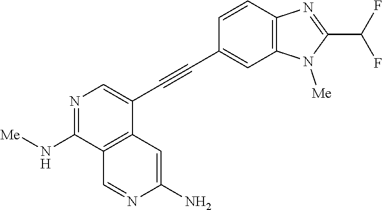 [SMILES: CNc1ncc(C#Cc2ccc3nc(C(F)F)n(C)c3c2)c2cc(N)ncc12]

To a solution of N-(5-((2-(difluoromethyl)-1-methyl-1H-benzo[d]imidazol-6-yl)ethynyl)-8-(methylamino)-2,7-naphthyridin-3-yl)cyclopropanecarboxamide (300.0 mg; 0.672 mmol; 1.00 eq.) Example 739, in a mixture solvent of DMSO/MeOH (1:5, 12 mL) was added a solution of NaOH (268.7 mg; 6.720 mmol; 10.00 eq.) in water (3 mL). The mixture was stirred at 70° C. overnight. The desired product was detected via LCMS. The mixture was allowed to cool to room temperature, the precipitated solids were collected by filtration and washed with water (3×5 mL). The solids were dried under vacuum to afford 4-((2-(difluoromethyl)-1-methyl-1H-benzo[d]imidazol-6-yl)ethynyl)-N¹-methyl-2,7-naphthyridine-1,6-diamine as a yellow solid (179.6 mg, 70.2%). LCMS (ESI) m/z 379.1, [M+H]⁺.

### Step 4: (1S,2R)—N-(5-((2-(difluoromethyl)-1-methyl-1H-benzo[d]imidazol-6-yl)ethynyl)-8-(methylamino)-2,7-naphthyridin-3-yl)-2-methylcyclopropane-1-carboxamide

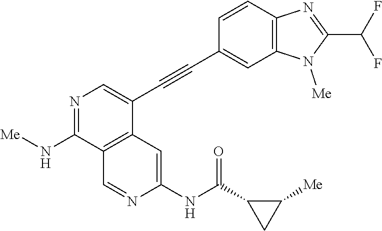 [SMILES: CNc1ncc(C#Cc2ccc3nc(C(F)F)n(C)c3c2)c2cc(NC(=O)[C@H]3C[C@H]3C)ncc12]

To a solution of 4-((2-(difluoromethyl)-1-methyl-1H-benzo[d]imidazol-6-yl)ethynyl)-N¹-methyl-2,7-naphthyridine-1,6-diamine (80.0 mg; 0.211 mmol; 1.00 eq.) and (1S,2R)-2-methylcyclopropane-1-carboxylic acid (19.0 mg; 0.190 mmol; 0.90 eq.) in pyridine (4 mL) was added POCl₃ (97.2 mg; 0.633 mmol; 3.00 eq.) at 0° C. under nitrogen atmosphere. The mixture was stirred at room temperature for 5 minutes. The desired product was detected via LCMS. The resulting mixture was diluted with EtOAc (25 mL), washed with water (2×5 mL) and dried over anhydrous Na₂SO₄. The organic layer was concentrated under vacuum. The residue was purified by flash chromatography on silica gel column using 0-4% MeOH in CH₂Cl₂ as eluent to provide (1S,2R)—N=(5-((2-(difluoromethyl)-1-methyl-1H-benzo[d]imidazol-6-yl)ethynyl)-8-(methylamino)-2,7-naphthyridin-3-yl)-2-methylcyclopropane-1-carboxamide as a yellow solid (26.1 mg, 25.6%). LCMS (ESI) m/z 461.2, [M+H]⁺. ¹H NMR (400 MHz, DMSO-d₆) δ 11.02 (s, 1H), 9.38 (s, 1H), 8.80 (s, 1H), 8.38-8.32 (m, 1H), 8.30 (s, 1H), 8.04 (s, 1H), 7.80 (d, J=8.4 Hz, 1H), 7.52 (d, J=8.4 Hz, 1H), 7.44 (t, J=52.0 Hz, 1H), 3.99 (s, 3H), 3.03 (d, J=4.4 Hz, 3H), 2.1.8-2.08 (m, 1H), 1.39-1.29 (m, 1H), 1.17 (d, J=6.0 Hz, 3H), 1.07-0.99 (m, 1H), 0.91-0.84 (m, 1H).

### Examples 776 and 777: Synthesis of (1S,2R)—N-(5-((4-((1-acetylazetidin-3-yl)oxy)phenyl)ethynyl)-8-(methylamino)-2,7-naphthyridin-3-yl)-2-(difluoromethyl)cyclopropane-1-carboxamide (Example 776) and (1R,2S)—N-(5-((4-((1-acetylazetidin-3-yl)oxy)phenyl)ethynyl)-8-(methylamino)-2,7-naphthyridin-3-yl)-2-(difluoromethyl)cyclopropane-1-carboxamide (Example 777)

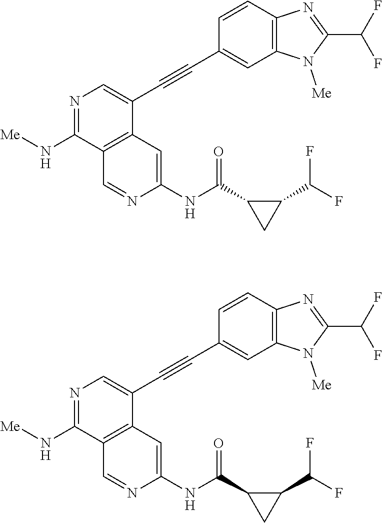 [SMILES: CNc1ncc(C#Cc2ccc3nc(C(F)F)n(C)c3c2)c2cc(NC(=O)[C@H]3C[C@H]3C(F)F)ncc12 ; CNc1ncc(C#Cc2ccc3nc(C(F)F)n(C)c3c2)c2cc(NC(=O)[C@@H]3C[C@@H]3C(F)F)ncc12]

To a stirred solution of 1-(3-(4-((6-amino-1-(methylamino)-2,7-naphthyridin-4-yl)ethynyl)phenoxy)azetidin-1-yl)ethan-1-one (Example 760, step 4) (130.0 mg; 0.336 mmol; 1.00 eq.) and 2-(difluoromethyl)cyclopropane-1-carboxylic acid (cis racemate) (41.1 mg; 0.302 mmol; 0.90 eq.) in pyridine (6 mL) was added POCl₃ (154.3 mg; 1.006 mmol; 3.00 eq.) dropwise at 0° C. The resulting mixture was stirred at room temperature for 0.5 hour. The desired product was detected via LCMS. The reaction was quenched by the addition of water (1.0 mL) and extracted with EtOAc (3×50 mL). The combined organic layers were washed with brine (3×10 mL) and dried over anhydrous Na₂SO₄. After filtration, the filtrate was concentrated under reduced pressure. The residue was purified by flash chromatography on silica gel column using 2-10% of MeOH in CH₂Cl₂ as eluent to afford the cis racemate. The cis racemate was separated by Prep-Chiral-HPLC (Column: CHIRALPAK IG, 2×25 cm, 5 μm; Mobile Phase A: Hex (0.5% 2 M NH₃-MeOH), Mobile Phase B: EtOH:DCM=1:1; Flow rate: 20 mL/min; Gradient: 40% B to 40% B in 22 min; Wave Length: 220/254 nm; R_{T1}(min): 15.78; R_{T2}(min): 20.8) to afford (1S,2R)—N-(5-((4-((1-acetylazetidin-3-yl)oxy)phenyl)ethynyl)-8-(methylamino)-2,7-naphthyridin-3-yl)-2-(difluoromethyl)cyclopropane-1-carboxamide (Example 776, the faster peak) as a yellow solid (23.5 mg, 13.7%) and (1R,2S)N-(5-((4-((1-acetylazetidin-3-yl)oxy)phenyl)ethynyl)-8-(methylamino)-2,7-naphthyridin-3-yl)-2-(difluoromethyl)cyclopropane-1-carboxamide (Example 777, the slower peak) as a yellow solid (23.3 mg, 13.5%). The two configurations are arbitrarily assigned. LCMS (ESI) m/z 506.2, [M+H]⁺.

HNMR for Example 776: ¹H NMR (400 MHz; DMSO-d₆) δ 11.32 (s, 1H), 9.39 (s, 1H), 8.62 (s, 1H), 8.40-8.30 (m, 1H), 8.30-8.27 (s, 1H), 7.53 (d, J=8.4 Hz, 2H), 6.93 (d, J=8.4 Hz, 2H), 6.30-5.90 (m, 1H), 5.16-5.01 (m, 1H), 4.67-4.49 (m, 1H), 4.39-4.20 (m, 1H), 4.20-4.02 (m, 1H), 3.88-3.70 (m, 1H), 3.02 (d, J=4.4 Hz, 3H), 2.47-2.39 (m, 1H), 2.00-1.88 (m, 1H), 1.81 (s, 3H), 1.42-1.29 (m, 2H).

HNMR for Example 777: ¹H NMR (400 MHz, DMSO-d₆) δ 11.32 (s, 1H), 9.39 (s, 1H), 8.62 (s, 1H), 8.42-8.30 (m, 1H), 8.30-8.13 (s, 1H), 7.53 (d, J=8.4 Hz, 2H), 6.93 (d, J=8.4 Hz, 2H), 6.28-5.90 (m, 1H), 5.14-5.00 (m, 1H); 4.70-4.53 (m, 1H), 4.40-4.27 (m, 1H), 4.18-4.04 (in, 1H), 3.83-3.62 (m, 1H), 3.02 (d, J=4.4 Hz, 3H), 2.48-2.39 (m, 1H), 2.02-1.85 (m, 1H), 1.81 (s, 3H), 1.42-1.30 (m, 2H).

### Example 778: Synthesis of N-(5-((5-methoxypyridin-2-yl)ethynyl)-8-((methyl-d₃-naphthyridin-3-yl)cyclopropanecarboxamide

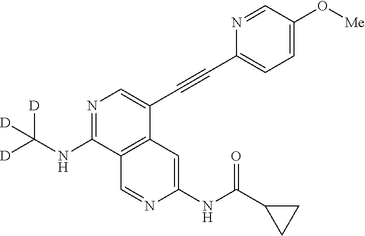 [SMILES: [2H]C([2H])([2H])Nc1ncc(C#Cc2ccc(OC)cn2)c2cc(NC(=O)C3CC3)ncc12]

To a stirred solution of 2-ethynyl-5-methoxypyridine (164.2 mg; 1.234 mmol; 2.00 eq.) and N-(5-bromo-8-((methyl-d₃)amino)-2,7-naphthyridin-3-yl)cyclopropanecarboxamide (200.0 mg; 0,617 mmol; 1.00 eq.) in DMF (2 mL) were added XPhos Pd G₃ (52.2 mg; 0.062 mmol; 0.10 eq.), XPhos (58.8 mg; 0.123 mmol; 0.20 eq.), Et₃N (312.1 mg; 3.085 mmol; 5.00 eq) and CuI (11.7 mg; 0.062 mmol; 0.10 eq.). The resulting mixture was stirred at 110° C. for 1 hour under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide N-(5-((5-methoxypyridin-2-yl)ethynyl)-8-((methyl-d₃)amino)-2,7-naphthyridin-3-yl)cyclopropanecarboxamide as a light brown solid (87.7 mg, 36.0%). LCMS (ESI) m/z 377.2, [M+H]⁺. ¹H NMR. (400 MHz, DMSO-d₆) δ 11.10 (s, 1H), 9.39 (s, 1H), 8.63 (s, 1H), 8.41 (s, 1H), 8.32 (d, J=3.2 Hz, 1H), 8.30 (s, 1H), 7.60 (d, J=8.8 Hz, 1H), 7.48 (dd, J=8.8, 3.2 Hz, 1H), 3.89 (s, 3H), 2.12-2.03 (m, 1H), 0.93-0.81 (m, 4H).

### Example 779: Synthesis of (1S,2R)—N-(5-((5-methoxypyridin-2-yl)ethynyl)-8-((methyl-d₃)amino)-2,7-naphthyridin-3-yl)-2-methylcyclopropane-1-carboxamide

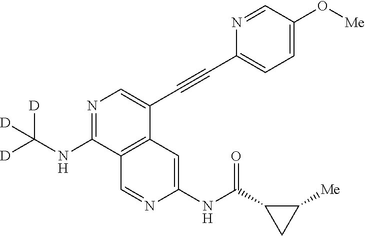 [SMILES: [2H]C([2H])([2H])Nc1ncc(C#Cc2ccc(OC)cn2)c2cc(NC(=O)[C@H]3C[C@H]3C)ncc12]

### Step 1: 4-((5-methoxypyridin-2-yl)ethynyl) N¹-(methyl-d₃)-2,7-naphthyridine-1,6-diamine

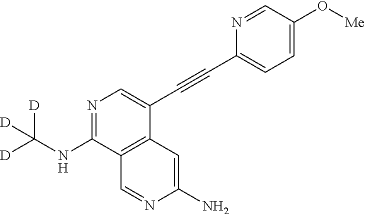 [SMILES: [2H]C([2H])([2H])Nc1ncc(C#Cc2ccc(OC)cn2)c2cc(N)ncc12]

To a solution of N-(5-((5-methoxypyridin-2-yl)ethynyl)-8-((methyl-d₃)amino)-2,7-naphthyridin-3-yl)cyclopropanecarboxamide (Example 778) (110.0 mg; 0.292 mmol; 1.00 eq.) in a mixture solvent of MeOH/DMSO (5:1, 2.4 mL) was added a solution of NaOH (116.8 mg; 2.920 mmol; 10.00 eq.) in water (0.4 mL) at 0° C. The mixture was stirred at 60° C. overnight. The desired product was detected via LCMS. The mixture was allowed to cool down to room temperature. The MeOH was removed under reduced pressure. The mixture was purified by flash chromatography on pre-packed C18 column using 20-60% of MeCN in water (10 mmol/L NH₄HCO₃) as eluent to provide 4-((5-methoxypyridin-2-yl)ethynyl)-N¹-(methyl-d₃)-2,7-naphthyridine-1,6-diamine as a yellow green solid (80.0 mg, 88.8%). LCMS (ESI) m/z 309.1, [M+H]⁺.

### Step 2: (1S,2R)—N-(5-((5-methoxypyridin-2-yl)ethynyl)-8-((methyl-d₃)amino)-2,7-naphthyridin-3-yl)-2-methylcyclopropane-1-carboxamide

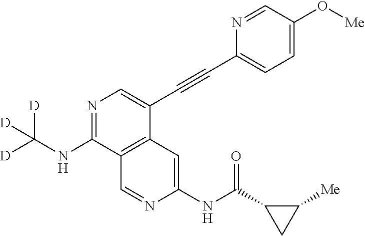 [SMILES: [2H]C([2H])([2H])Nc1ncc(C#Cc2ccc(OC)cn2)c2cc(NC(=O)[C@H]3C[C@H]3C)ncc12]

To a stirred solution of 4-((5-methoxypyridin-2-yl)ethynyl)-N¹-(methyl-d₃)-2,7-naphthyridine-1,6-diamine (80.0 mg; 0.259 mmol; 1.00 eq.) and (1S,2R)-2-methylcyclopropane-1-carboxylic acid (22.1 mg; 0.220 mmol; 0.85 eq.) in pyridine (2 mL) was added POCl₃ (119.3 mg; 0.777 mmol; 3.00 eq.) dropwise at 0° C. The resulting mixture was stirred at room temperature for 0.5 hour under nitrogen atmosphere. The desired product was detected via LCMS. The resulting mixture was diluted with water (5 mL), the mixture was neutralized to pH=7 with saturated NaHCO₃ solution. The resulting mixture was extracted with EtOAc (3×10 mL). The combined organic layers were washed with brine (3×10 mL), dried over anhydrous Na₂SO₄ and concentrated under reduced pressure. The residue was purified by flash chromatography on silica gel column using 5-8% of MeOH: in CH₂Cl₂ as eluent to provide (1S,2R)—N-(5-((5-methoxypyridin-2-yl)ethynyl)-8-((methyl-d₃)amino)-2,7-naphthyridin-3-yl)-2-1.5 methylcyclopropane-1-carboxamide as a light yellow solid (57.0 mg, 56.1%). LCMS (ESI) m/z 391.2, [M+H]⁺. ¹H NMR (400 MHz, DMSO-d₆) δ 10.97 (s, 1H), 9.37 (s, 1H), 8.62 (s, 1H), 8.34-8.30 (m, 3H), 7.62 (d, J=8.8 Hz, 1H), 7.47 (dd, J=8.8, 3.2 Hz, 1H), 3.88 (s, 3H), 2.12-2.05 (m, 1H), 1.35-1.26 (m, 1H), 1.15 (d, J=6.0 Hz, 3H), 1.03-0.96 (m, 1H), 0.89-0.82 (m, 1H).

### Example 780: Synthesis of N-(6-ethyl-5-((4-methoxyphenyl)ethynyl)-8-(methylamino)-2,7-naphthyridin-3-yl)cyclopropanecarboxamide

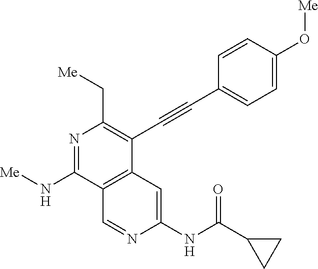 [SMILES: CCc1nc(NC)c2cnc(NC(=O)C3CC3)cc2c1C#Cc1ccc(OC)cc1]

### Step 1: 1-(tert-butyl) 3-methyl 2-(2-chloro-5-(methoxycarbonyl)pyridin-4-yl)malonate

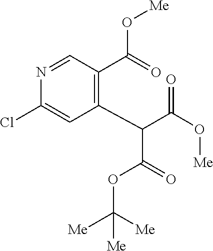 [SMILES: COC(=O)c1cnc(Cl)cc1C(C(=O)OC)C(=O)OC(C)(C)C]

To a stirred solution of methyl 4,6-dichloronicotinate (5 g; 24.26 mmol; 1.00 eq.) and tert-butyl methyl malonate (6.5 g; 37.314 mmol; 1.54 eq.) in DMF (50 mL) was added Cs₂CO₃ (16 g; 49.10 mmol; 2.02 eq.). The resulting solution was stirred at 40° C. for 3 hours. The desired product was detected via LCMS. The resulting mixture was diluted with water (250 mL), extracted with EtOAc (2×100 mL) and the organic layers were combined. The organic layer was dried over anhydrous sodium sulfate and concentrated under vacuum to provide 1-(tert-butyl) 3-methyl 2-(2-chloro-5-(methoxycarbonyl)pyridin-4-yl)malonate as a yellow oil (10.7 g, crude). LCMS (ESI) m/z 344.1, [M+H]⁺.

### Step 2: methyl 6-chloro-4-(2-methoxy-2-oxoethyl)nicotinate

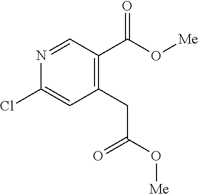 [SMILES: COC(=O)Cc1cc(Cl)ncc1C(=O)OC]

To a stirred solution of 1-(tert-butyl) 3-methyl 2-(2-chloro-5-(methoxycarbonyl)pyridin-4-yl)malonate (10 g; 30.32 mmol; 1.00 eq.) in CH₂Cl₂ (50 mL) was added TFA (20 mL). The resulting solution was stirred at room temperature for 12 hours. The reaction was concentrated under vacuum. The reaction mixture was diluted with water (300 mL). The mixture was neutralized to pH=7 with saturated NaHCO₃ solution and extracted with EtOAc (2×200 mL). The combined organic layer was dried over anhydrous sodium sulfate and concentrated under vacuum to provide methyl 6-chloro-4-(2-methoxy-2-oxoethyl)nicotinate as an off-white solid (5 g, crude). LCMS (ESI) m/z 244.0, [M+H]⁺.

### Step 3: 6-chloro-2,7-naphthyridine-1,3(2H,4H)-dione

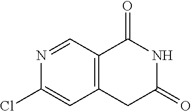 [SMILES: O=C1Cc2cc(Cl)ncc2C(=O)N1]

A solution of methyl 6-chloro-4-(2-methoxy-2-oxoethyl)nicotinate (5 g; 10.260 mmol; 1.00 eq.) in NH₃·H₂O (25 mL) was stirred at 120° C. for 12 hours in a sealed tube. After the reaction was completed, the mixture was filtered and the filter cake was washed with water to provide 6-chloro-2,7-naphthyridine-1,3(2H,4H)-dione as a brown solid (1.6 g, crude). LCMS (ESI) m/z 197.0, [M+H]⁺. ¹H NMR (400 MHz, DMSO-d₆) δ 9.65 (s, 1H), 8.27 (s, 1H), 6.56 (s, 1H), 4.73 (s, 2H).

### Step 4: N-(6,8-dioxo-5,6,7,8-tetrahydro-2,7-naphthyridin-3-yl)cyclopropanecarboxamide

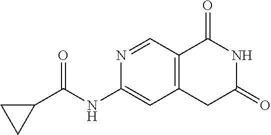 [SMILES: O=C1Cc2cc(NC(=O)C3CC3)ncc2C(=O)N1]

To a solution of Pd₂(dba)₃ (303 mg; 0.331 mmol; 0.10 eq.), XantPhos (383 mg; 0.662 mmol; 0.20 eq.) and Cs₂CO₃ (2.16 g; 6.63 mmol; 2.00 eq.) in 1,4-dioxane (30 mL) was added cyclopropanecarboxamide (1.12 g; 13.1 mmol; 4.00 eq.) and 6-chloro-2,7-naphthyridine-1,3(2H,4H)-dione (650 mg; 3.31 mmol; 1.00 eq.). The resulting solution was stirred at 130° C. for 48 hours under nitrogen atmosphere. The desired product was detected via LCMS. The reaction mixture was concentrated under vacuum and purified by flash chromatography on silica gel column using 10-20% of MeOH in CH₂Cl₂ as eluent to provide N-(6,8-dioxo-5,6,7,8-tetrahydro-2,7-naphthyridin-3-yl)cyclopropanecarboxamide as a brown solid (500 mg, 61%). LCMS (ESI) m/z 246.1, [M+H]⁺.

### Step 5: 6-(cyclopropanecarboxamido)-2,7-naphthyridine-1,3-diyl bis(trifluoromethanesulfonate)

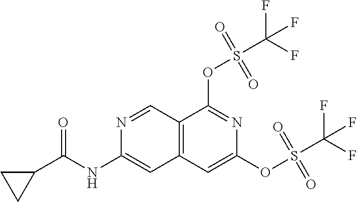 [SMILES: O=C(Nc1cc2cc(OS(=O)(=O)C(F)(F)F)nc(OS(=O)(=O)C(F)(F)F)c2cn1)C1CC1]

To a stirred solution of N-(6,8-dioxo-5,6,7,8-tetrahydro-2,7-naphthyridin-3-yl)cyclopropanecarboxamide (400 mg; 1.63 mmol; 1.00 eq.) and pyridine (400 mg; 5.057 mmol; 3.10 eq.) in DCE (40 mL) was added a solution of Tf₂O (1.4 g; 4.96 mmol; 3.00 eq.) in DCE (5 mL) dropwise at 0° C. under nitrogen atmosphere. The resulting solution was stirred at 0° C. for 1 hour. The desired product was detected via LCMS. The reaction was diluted with CH₂Cl₂ (100 mL) and washed with saturated NaCl solution (3×5 mL). The organic phase was dried with Na₂SO₄, filtered and the solvent was removed under reduced pressure. The residue was purified by flash chromatography on pre-packed C18 column using 50-80% MeCN in water (10 mmol/L NH₄HCO₃) as eluent to provide 6-(cyclopropanecarboxamido)-2,7-naphthyridine-1,3-diyl bis(trifluoromethanesulfonate) as a off-white solid (450 mg, 54.0%). LCMS (ESI) m/z 510.0, [M+H]⁺.

### Step 6: N-(6-methyl-8-(methylamino)-2,7-naphthyridin-3-yl)cyclopropanecarboxamide

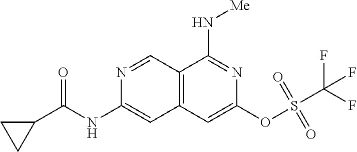 [SMILES: CNc1nc(OS(=O)(=O)C(F)(F)F)cc2cc(NC(=O)C3CC3)ncc12]

To a solution of 6-(cyclopropanecarboxamido)-2,7-naphthyridine-1,3-diyl bis(trifluoromethanesulfonate) (400 mg; 0.786 mmol; 1.00 eq.) and methanamine hydrochloride (60 mg; 0.888 mmol; 1.13 eq.) in NMP (10 mL) was added DIPEA (100 mg; 0.774 mmol; 1.97 eq.). The resulting solution was stirred at room temperature for 2 hours. The desired product was detected via LCMS. The reaction was diluted with EtOAc (80 mL), washed with saturated NaCl solution (3×5 mL). The organic phase was dried over Na₂SO₄, filtered and the solvent was removed under reduced pressure. The residue was purified by flash chromatography on silica gel column using 60-80% of EtOAc in petroleum ether as eluent to provide N-(6-methyl-8-(methylamino)-2,7-naphthyridin-3-yl)cyclopropanecarboxamide as a white solid (183 mg, 58%). LCMS (ESI) m/z 391.1 [M+H]⁺. ¹H NMR (400 MHz, DMSO-d₆) δ 11.09 (s, 1H), 9.37 (s, 1H), 8.64-8.58 (m, 1H), 8.29 (s, 1H), 6.83 (s, 1H), 2.94 (d, J=4.4 Hz, 3H), 2.10-2.01 (m, 1H), 0.88-0.81 (m, 4H).

### Step 7: N-(8-(methylamino)-6-vinyl-2,7-naphthyridin-3-yl)cyclopropanecarboxamide

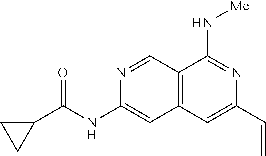 [SMILES: C=Cc1cc2cc(NC(=O)C3CC3)ncc2c(NC)n1]

To a solution of Pd(DtBPF)Cl₂ (16.8 mg; 0.026 mmol; 0.10 eq.) and K₃PO₄ (108 mg; 0.508 mmol; 1.99 eq.) in a mixture solvent of 1,4-dioxane/water (5:1, 4.8 mL) was added 4,4,5,5-tetramethyl-2-vinyl-1,3,2-dioxaborolane (48 mg; 0.312 mmol; 1.22 eq.) and 6-cyclopropaneamido-1-(methylamino)-2,7-naphthyridin-3-yltrifluoromethanesulfonate (200 mg; 0.256 mmol; 1.00 eq.). The resulting solution was stirred at 90° C. for 2 hours under nitrogen atmosphere. The desired product was detected via LCMS. The reaction was concentrated under vacuum. The residue was purified by flash chromatography on silica gel column using 60-80% of EtOAc in petroleum ether as eluent to afford N-(8-(methylamino)-6-vinyl-2,7-naphthyridin-3-yl)cyclopropanecarboxamide as a white solid (87 mg, 64.0%). LCMS (ESI) m/z 269.1, [M+H]⁺.

### Step 8: N-(6-ethyl-8-(methylamino)-2,7-naphthyridin-3-yl)cyclopropanecarboxamide

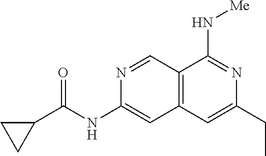 [SMILES: CCc1cc2cc(NC(=O)C3CC3)ncc2c(NC)n1]

To a solution of N-(8-(methylamino)-6-vinyl-2,7-naphthyridin-3-yl)cyclopropanecarboxamide (87 mg; 0.323 mmol; 1.00 eq.) in MeOH (5 mL) was added 10% Pd/C (87 mg; 100% w/w) under nitrogen atmosphere. The mixture was hydrogenated at 35° C. for 3 hours under hydrogen atmosphere (2 atm). The desired product was detected via LCMS. After filtration, the filtrate was concentrated under vacuum. The residue was purified by flash chromatography on pre-packed C18 column using 50-80% MeCN in water (10 mmol/L NH₄HCO₃) as eluent to provide N-(6-ethyl-8-(methylamino)-2,7-naphthyridin-3-yl)cyclopropanecarboxamide as a white solid (60.5 mg, 68.4%). LCMS (ESI) m/z 271.0, [M+H]⁺.

### Step 9: N-(6-ethyl-5-iodo-8-(methylamino)-2,7-naphthyridin-3yl)cyclopropanecarboxamide

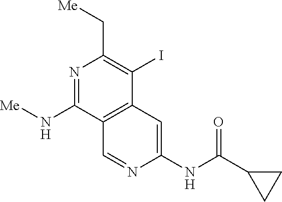 [SMILES: CCc1nc(NC)c2cnc(NC(=O)C3CC3)cc2c1I]

A solution of N-(6-ethyl-8-(methylamino)-2,7-naphthyridin-3-yl)cyclopropanecarboxamide (60.0 mg; 0.222 mmol; 1.00 eq.) in DMF (3 mL). To the above solution was added and MS (49.9 mg; 0.222 mmol; 1.00 eq.) at 0° C. under nitrogen atmosphere. The resulting solution was stirred at 0° C. for 1 hour. The desired product was detected via LCMS. The resulting mixture was diluted with water (15 mL) and extracted with EtOAc (3×10 mL). The combined organic layers were washed with brine (2×10 mL), dried over anhydrous Na₂SO₄. After filtration, the filtrate was concentrated under reduced pressure. The residue was purified by flash chromatography on silica gel column using 30-40% of EtOAc in petroleum ether as eluent to provide N-(6-ethyl-5-iodo-8-(methylamino)-2,7-naphthyridin-3yl)cyclopropanecarboxamide as a yellow solid (80 mg, 90.9%). LCMS (ESI) m/z 397.0, [M+H]⁺.

### Step 10: N-(6-ethyl-5-((4-methoxyphenyl)ethynyl)-5(methylamino)-2,7-naphthyridin-3-yl)cyclopropanecarboxamide

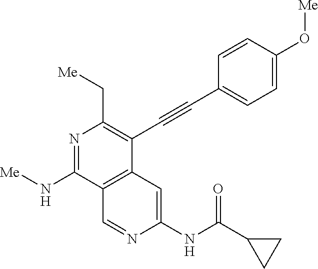 [SMILES: CCc1nc(NC)c2cnc(NC(=O)C3CC3)cc2c1C#Cc1ccc(OC)cc1]

To a solution of 1-ethynyl-4-methoxybenzene (100.1 mg; 0.757 mmol; 5.00 eq.) and N-(6-ethyl-5-iodo-8-(methylamino)-2,7-naphthyridin-3yl)cyclopropanecarboxamide (60.0 mg; 0.151 mmol; 1.00 eq.) in DMA (5 mL) were added Pd(PPh₃):1 (35.0 mg; 0.030 mmol; 0.20 eq.), CuI (5.7 mg; 0.030 mmol; 0.20 eq.) and Et₃N (107.3 mg; 1.060 mmol; 7.00 eq.) under nitrogen atmosphere. The resulting solution was stirred at 60° C. for 5 hours. The desired product was detected via LCMS. The mixture was allowed to cool down to room temperature. The resulting mixture was diluted with water (10 mL) and extracted with EtOAc (4×10 mL). The combined organic layers were washed with brine (3×10 mL) and dried over anhydrous Na₂SO₄. After filtration, the filtrate was concentrated under reduced pressure. The residue was purified by flash chromatography on silica gel column using 1-6% of MeOH in CH₂Cl₂ as eluent to provide the desired product. The product was further purified by Prep-Achiral-SFC (Column: GreenSep Naphthyl, 3×25 cm, 5 μm; Mobile Phase A: CO₂, Mobile Phase B: MeOH (0.1% 2 M NH₃-MeOH); Flow rate: 75 mL/min; Gradient: isocratic 31% B; Wave Length: 254 nm) to provide N-(6-ethyl-5-((4-methoxyphenyl)ethynyl)-8-(methylamino)-2,7-naphthyridin-3-yl)cyclopropanecarboxamide as a yellow solid (10.4 mg, 17.2%). LCMS (ESI) m/z 401.2, [M+H]⁺. ¹H NMR (400 MHz, DMSO-d₆) δ 10.98 (s, 1H), 9.30 (s, 1H), 8.65 (s, 1H), 8.22-8.12 (m, 1H), 7.55-7.43 (m, 2H), 7.07-6.96 (m, 2H), 3.81 (s, 3H), 3.04 (d, J=4.4 Hz, 3H), 2.97-2.92 (m, 2H), 2.11-2.04 (m, 1H), 1.30 (t, J=7.2 Hz, 3H), 0.91-0.81 (m, 4H).

### Example 781: Synthesis of N-(5-((4-methoxyphenyl)ethynyl)-6-methyl-8-(methylamino)-2,7-naphthyridin-3-yl)cyclopropanecarboxamide

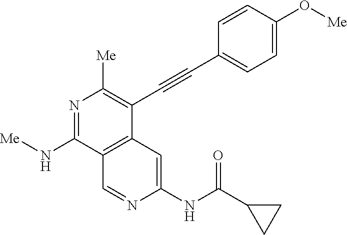 [SMILES: CNc1nc(C)c(C#Cc2ccc(OC)cc2)c2cc(NC(=O)C3CC3)ncc12]

### Step 1: N-(6-methyl-8-(methylamino)-2,7-naphthyridin-3-yl)cyclopropanecarboxamide

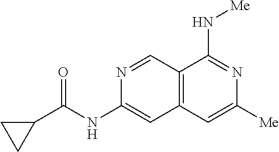 [SMILES: CNc1nc(C)cc2cc(NC(=O)C3CC3)ncc12]

A solution of 6-(cyclopropanecarboxamido)-1-(methylamino)-2,7-naphthyridin-3-yl trifluoromethanesulfonate (Example 780, step 6) (200.0 mg; 0.512 mmol; 1.00 eq.) and 2,4,6-trimethyl-1,3,5,2,4,6-trioxatriborinane (192.9 mg; 1.537 mmol; 3.00 eq.) in a mixture solvent of 1,4-dioxane/water (5:1, 1.2 mL). To the above solution were added Pd(DtBPF)Cl₂ (66.7 mg; 0.102 mmol; 0.20 eq.) and K₃PO₄ (217.5 mg; 1.025 mmol; 2.00 eq.) under nitrogen atmosphere. The resulting solution was stirred at 90° C. for 2 hours. The desired product was detected via LCMS. The resulting mixture was concentrated under reduced pressure and purified by flash chromatography on silica gel column using 40-60% of EtOAc in petroleum ether as eluent to provide N-(6-methyl-8-(methylamino)-2,7-naphthyridin-3-yl)cyclopropanecarboxamide as a off-white solid (110 mg, 83.7%). LCMS (ESI) m/z 257.1, [M+H]⁺.

### Step 2: N-(5-iodo-6-methyl-8-(methylamino)-2,7-naphthyridin-3-yl)cyclopropanecarboxamide

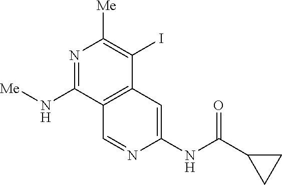 [SMILES: CNc1nc(C)c(I)c2cc(NC(=O)C3CC3)ncc12]

A solution of N-(6-methyl-8-(methylamino)-2,7-naphthyridin-3-yl)cyclopropanecarboxamide (160.0 mg; 0.624 mmol; 1.00 eq.) and NIS (140.4 mg; 0.624 mmol; 1.00 eq.) in DMF (6 mL) was stirred at 0° C. for 1 hour under nitrogen atmosphere. The desired product was detected via LCMS. The resulting mixture was diluted with water (10 mL) and extracted with EtOAc (3×10 mL). The combined organic layers were washed with brine (2×10 mL), dried over anhydrous Na₂SO₄. After filtration, the filtrate was concentrated under reduced pressure. The residue was purified by flash chromatography on silica gel column using 50-60% of EtOAc in petroleum ether as eluent to provide N-(5-iodo-6-methyl-8-(methylamino)-2,7-naphthyridin-3-yl)cyclopropanecarboxamide as a yellow solid (220 mg, 92%). LCMS (ESI) m/z 383.0, [M+H]⁺.

### Step 3: N-(5-((4-methoxyphenyl)ethynyl)-6-methyl-5(methylamino)-2,7-naphthyridin-3-yl)cyclopropanecarboxamide

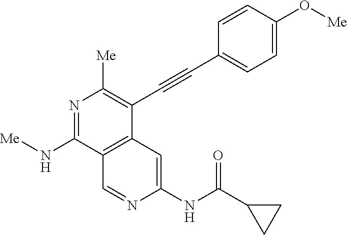 [SMILES: CNc1nc(C)c(C#Cc2ccc(OC)cc2)c2cc(NC(=O)C3CC3)ncc12]

To a solution of 1-ethynyl-4-methoxybenzene (172.8 mg; 1.307 mmol; 5.00 eq.) and N-(5-iodo-6-methyl-8-(methylamino)-2,7-naphthyridin-3-yl)cyclopropanecarboxamide (100.0 mg; 0.262 mmol; 1.00 eq.) in DMA (10 mL) were added Pd(PPh₃)₄ (60.4 mg; 0.052 mmol; 0.20 eq.), Et₃N (185.3 mg; 1.831 mmol; 7.00 eq.) and CuI (9.9 mg; 0.052 mmol; 0.20 eq.) under nitrogen atmosphere. The resulting solution was stirred at 60° C. for 4 hours. The desired product was detected via LCMS. The mixture was allowed to cool down to room temperature. The resulting mixture was diluted with water (10 mL) and extracted with EtOAc (4×10 mL). The combined organic layers were washed with brine (3×10 mL), dried over anhydrous Na₂SO₄. After filtration, the filtrate was concentrated under reduced pressure. The residue was purified by flash chromatography on silica gel column using 1-6% of MeOH in CH₂Cl₂ as eluent to provide the desired product. The product was further purified by Prep-Achiral-SFC (Column: DAICEL DCpak P4VP 3×25 cm, 5 μm; Mobile Phase A: CO₂, Mobile Phase B: MeCN:MeOH=4:1(0.1% 2 M NH₃-MeOH); Flow rate: 60 mL/min; Gradient: isocratic 50% B; Wave Length: 254 nm; Rr (min): 12.75) to provide N-(5-((4-methoxyphenyl)ethynyl)-6-methyl-8-(methylamino)-2,7-naphthyridin-3-yl)cyclopropanecarboxamide as a yellow solid (10.2 mg, 10.1%). LCMS (ESI) m/z 387.2, [M+H]⁺. ¹H NMR (400 MHz, DMSO-d₆) δ 10.99 (s, 1H), 9.30 (s, 1H), 8.63 (s, 1H), 8.21-8.14 (m, 1H), 7.50 (d, J=8.4 Hz, 2H), 7.01 (d, J=8.4 Hz, 2H), 3.81 (s, 3H), 3.01 (d, J=4.4 Hz, 3H), 2.60 (s, 3H), 2.12-2.03 (m, 1H), 0.93-0.81 (m, 4H).

### Example 782: Synthesis of (E)-N-(5-(4-(2-methoxyethoxy)styryl)-8-(methylamino)-2,7-naphthyridin-3-yl)cyclopropanecarboxamide

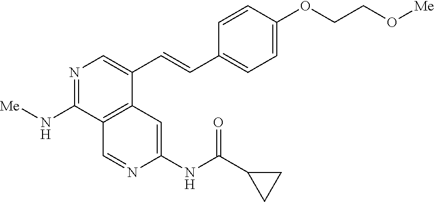 [SMILES: CNc1ncc(/C=C/c2ccc(OCCOC)cc2)c2cc(NC(=O)C3CC3)ncc12]

### Step 1: N-(8-(methylamino)-5-vinyl-2,7-naphthyridin-3-yl)cyclopropanecarboxamide

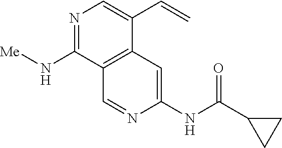 [SMILES: C=Cc1cnc(NC)c2cnc(NC(=O)C3CC3)cc12]

A solution of N-(5-bromo-8-(methylamino)-2,7-naphthyridin-3-yl)cyclopropanecarboxamide (2.00 g; 6.250 mmol; 1.00 eq.), 4,4,5,5-tetramethyl-2-vinyl-1,3,2-dioxaborolane (1.44 g; 9.375 mmol; 1.50 eq.), Pd(DtBPF)Cl₂ (405.6 mg; 0.625 mmol; 0.10 eq.) and K₃PO₄ (2.65 g; 12.50 mmol; 2.00 eq.) in a mixture solvent of 1,4-dioxane/water (5:1, 12 mL) was stirred at 90° C. for 2 hours under nitrogen atmosphere. The desired product was detected via LCMS. The solvent was concentrated under vacuum. The residue was purified by flash chromatography on silica gel column using 0-15% of MeOH in CH₂Cl₂ as eluent to provide the crude product. The crude product was purified by flash chromatography on pre-packed C18 column using 40-50% of MeCN in water (10 mmol/L NH₄HCO₃) as eluent to provide N-(8-(methylamino)-5-vinyl-2,7-naphthyridin-3-yl)cyclopropanecarboxamide as a yellow solid (1.35 g, 80.5%). LCMS (ESI) m/z 269.1, [M+H]⁺.

### Step 2: (E)-N-(5-(4-(2-methoxyethoxy)styryl)-8-(methylamino)-2,7-naphthyridin-3-yl)cyclopropanecarboxamide

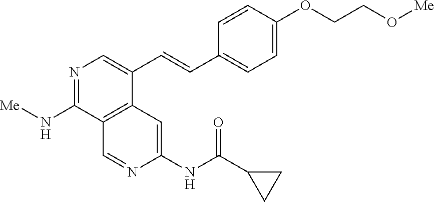 [SMILES: CNc1ncc(/C=C/c2ccc(OCCOC)cc2)c2cc(NC(=O)C3CC3)ncc12]

A solution of N-(8-(methylamino)-5-vinyl-2,7-naphthyridin-3-yl)cyclopropanecarboxamide (100.0 mg; 0.37 mmol; 1.00 eq.), 1-bromo-4-(2-methoxyethoxy)benzene (129.0 mg; 0.558 mmol; 1.50 eq.), Pd(PPh₃)₂Cl₂ (26.2 mg; 0.037 mmol; 0.10 eq.) and Na₂CO₃ (79.1 mg; 0.746 mmol; 2.00 eq.) in DMF (3 mL) was stirred at 100° C. for 14 hours under nitrogen atmosphere. The desired product was detected via LCMS. The mixture was purified by flash chromatography on pre-packed C18 column using 40-60% of MeCN in water (10 mmol/L NH₄HCO₃) as eluent to provide (E)₄¹-(5-(4-(2-triethoxyethoxy)styryl)-8-(methylamino)-2,7-naphthyridin-3-yl)cyclopropanecarboxamide as a yellow solid (45.0 mg, 28.8%). LCMS (ESI) m/z 419.2, [M+H]⁺. ¹H NMR (400 MHz, DMSO-d₆) δ 11.06 (s, 1H), 937 (s, 1H), 8.57 (s, 1H), 8.26 (s, 8.20-8.10 (m, 1H), 7.50 (d, J=8.4 Hz, 2H), 7.22 (d, J=16.0 Hz, 1H), 7.05 (d, J=16.0 Hz, 1H), 6.98 (d, J=8.4 Hz, 2H), 4.12 (t, J=4.4 Hz, 2H), 3.68 t, J=4.4 Hz, 2H), 3.33 (s, 3H), 3.02 (d, J=4.4 Hz, 3H), 2.14-2.02 (m, 1H), 0.92-0.80 (m, 4H).

### Example 783: Synthesis of (E)-N-(5-(4-(cyanomethyl)styryl)-8-(methylamino)-2,7-naphthyridin-3-yl)cyclopropanecarboxamide

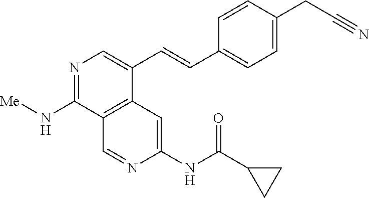 [SMILES: CNc1ncc(/C=C/c2ccc(CC#N)cc2)c2cc(NC(=O)C3CC3)ncc12]

A solution of N-(8-(methylamino)-5-vinyl-2,7-naphthyridin-3-yl)cyclopropanecarboxamide (100.0 mg; 0.373 mmol; 1.00 eq.), 2-(4-bromophenyl)acetonitrile (109.2 mg; 0.557 mmol; 1.49 eq.), Pd(PPh₃)₂Cl₂ (26.2 mg; 0.037 mmol; 0.10 eq.) and Na₂CO₃ (79.1 mg; 0.746 mmol; 2.00 eq.) in DMF (3 mL) was stirred at 100° C. for 14 hours under nitrogen atmosphere. The desired product was detected via LCMS. The resulting mixture was concentrated under reduced pressure. The residue was purified by flash chromatography on silica gel column using 5-15% of MeOH in CH₂Cl₂ as eluent to provide (E)-N-(5-(4-(cyanomethyl)styryl)-8-(methylamino)-2,7-naphthyridin-3-yl)cyclopropanecarboxamide as a yellow solid (13.9 mg, 9.0%). LCMS (ESI) m/z 384.2, [M+H]⁺. ¹H NMR (400 MHz, DMSO-d₆) δ 11.03 (s, 1H), 9.36 (s, 1H), 8.57 (s, 1H), 8.35 (s, 1H), 8.08-8.03 (m, 1H), 7.60 (d, J=8.4 Hz, 2H), 7.42 (d, J=16.4 Hz, 1H), 7.37 (d, J=8.4 Hz, 2H), 7.07 (d, J=16.4 Hz, 1H), 4.05 (s, 2H), 3.01 (d, J=4.4 Hz, 3H), 2.11-2.03 (m, 1H), 0.92-0.82 (m, 4H).

### Example 784 and 785: Synthesis of (E)-N-(5-(4-(2-hydroxy-2-methylpropoxy)styryl)-8-(methylamino)-2,7-naphthyridin-3-yl)cyclopropanecarboxamide (Example 784) and N-(5-(1-(4-(2-hydroxy-2-methyl propoxy)phenyl)vinyl)-8-(methylamino)-2,7-naphthyridin-3-yl)cyclopropanecarboxamide (Example 785)

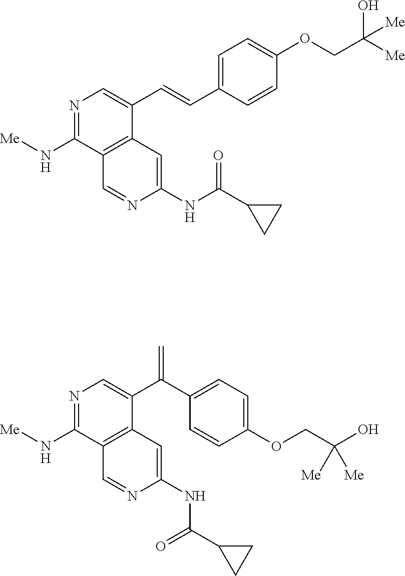 [SMILES: CNc1ncc(/C=C/c2ccc(OCC(C)(C)O)cc2)c2cc(NC(=O)C3CC3)ncc12 ; C=C(c1ccc(OCC(C)(C)O)cc1)c1cnc(NC)c2cnc(NC(=O)C3CC3)cc12]

A solution of N-(8-(methylamino)-5-vinyl-2,7-naphthyridin-3-yl)cyclopropanecarboxamide (92.0 mg; 0.343 mmol; 1.00 eq.), 1-(4-bromophenoxy)-2-methylpropan-2-ol (126.0 mg; 0.515 mmol; 1.50 eq.), Pd(PPh₃)₂Cl₂ (24.0 mg; 0.034 mmol; 0.10 eq.) and Na₂CO₃ (73.7 mg; 0.696 mmol; 2.03 eq.) in DMF (2.5 mL) was stirred at 100° C. for 14 hours under nitrogen atmosphere. The desired product was detected via LCMS. The resulting mixture was diluted with EtOAc (50 mL) and washed with brine (3×10 mL). The organic layer was dried over anhydrous Na₂SO₄. The resulting mixture was filtered and concentrated under reduced pressure. The residue was purified by flash chromatography on silica gel column using 0-10% of MeOH in CH₂Cl₂ as eluent to provide the crude product that was repurified by Prep-Achiral-SFC (Column: DAICEL DCpak P4VP 3×25 cm, 5 μm; Mobile Phase A: CO₂, Mobile Phase B: MeOH (0.1% 2 M NH₃-MeOH); Flow rate: 60 mL/min; Gradient: isocratic 47.0% B; Column Temperature (° C.): 35; Back Pressure (bar): 100; Wave Length: 220 nm) to provide (E)-N-(5-(4-(2-hydroxy-2-methylpropoxy)styryl)-8-(methylamino)-2,7-naphthyridin-3-yl)cyclopropanecarboxamide (Example 784) as a yellow solid (31.5 mg, 20.1%) and N-(5-(1-(4-(2-hydroxy-2-methyl propoxy)phenyl)vinyl)-8-(methylamino)-2,7-naphthyridin-3-yl)cyclopropanecarboxamide (Example 785) as a yellow solid (7.1 mg, 4.5%). LCMS (ESI) m/z 433.2, [M+H]⁺. HNMR for Example 784: ¹H NMR (400 MHz, DMSO-d₆) δ 11.01 (s, 1H), 9.35 (s, 1H), 8.55 (s, 1H), 8.30 (s, 1H), 8.01-7.94 (m, 1H), 7.49 (d, J=8.4 Hz, 2H), 7.22 (d, J=16.0 Hz, 1H), 7.00 (d, J=16.0 Hz, 1H), 6.97 (d, J=8.4 Hz, 2H), 4.63 (s, 1H), 3.75 (s, 2H), 3.00 (d, J=4.4 Hz, 3H), 2.14-2.00 (m, 1H), 1.22 (s, 6H), 0.95-0.81 (m, 4H).

### Example 786: Synthesis of N-(5-((E)-4-((2R,6S)-2,6-dimethylmorpholino)styryl)-8-(methylamino)-2,7-naphthyridin-3-yl)cyclopropanecarboxamide

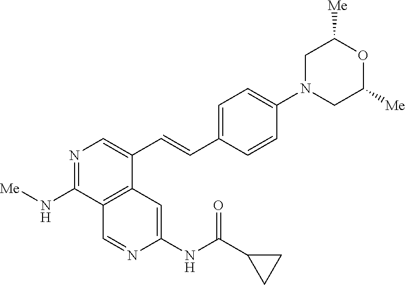 [SMILES: CNc1ncc(/C=C/c2ccc(N3C[C@@H](C)O[C@@H](C)C3)cc2)c2cc(NC(=O)C3CC3)ncc12]

### Step 1: (2R,6S)-4-(4-bromophenyl)-2,6-dimethylmorpholine

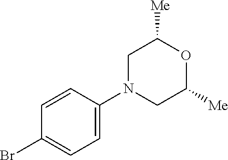 [SMILES: C[C@@H]1CN(c2ccc(Br)cc2)C[C@H](C)O1]

To a solution of (2R,6S)-2,6-dimethylmorpholine (500.0 mg; 4.341 mmol; 1.00 eq.) in toluene (8 mL) was added 1,4-dibromobenzene (2.03 g; 8.639 mmol; 1.99 eq.), Pd₂(dba)₃ (397.5 mg; 0.43 mmol; 0.10 eq.), BINAP (540.6 mg; 0.868 mmol; 0.20 eq.) and sodium 2-methylpropan-2-olate (584.1 mg; 6.07 mmol; 1.40 eq.). The mixture was stirred at 80° C. for 14 hours under nitrogen atmosphere. The desired product was detected via LCMS. The reaction was concentrated under pressure. The residue was purified by flash chromatography on silica gel column using 75-85% of EtOAc in petroleum ether as eluent to provide (2R,6S)-4-(4-bromophenyl)-2,6-dimethylmorpholine as a white solid (720.0 mg, 60.1%). LCMS (ESI) m/z 270.0, [M+H]⁺.

### Step 2: (2R,6S)-2,6-dimethyl-4-(4-vinylphenyl)morpholine

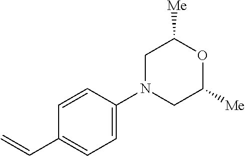 [SMILES: C=Cc1ccc(N2C[C@@H](C)O[C@@H](C)C2)cc1]

To a solution of (2R,6S)-4-(4-bromophenyl)-2,6-dimethylmorpholine (100.0 mg; 0.370 mmol; 1.00 eq.) in a mixture solvent of 1,4-dioxane/water (5:1, 3.6 mL) were added S-Phos (15.2 mg; 0.037 mmol; 0.10 eq.), K₃PO₄ (236.4 mg; 1.114 mmol; 3.01 eq.), Pd(OAc)₂ (4.2 mg; 0.019 mmol; 0.05 eq.) and 4,4,5,5-tetramethyl-2-vinyl-1,3,2-dioxaborolane (63.0 mg; 0.409 mmol; 1.11 eq.). The mixture was stirred at 80° C. for 14 hours under nitrogen atmosphere. The desired product was detected via LCMS. The reaction was concentrated under pressure and purified by flash chromatography on silica gel column using 80-90% of EtOAc in petroleum ether as eluent to provide (2R,6S)-2,6-dimethyl-4-(4-vinylphenyl)morpholine as a light yellow solid (70.0 mg, 80.2%). LCMS (ESI) m/z 218.1, [M+H]⁺.

### Step 3: N-(5-((E)-4-((2R,6S)-2,6-dimethylmorpholino)styryl)-8-(methylamino)-2,7-naphthyridin-3-yl)cyclopropanecarboxamide

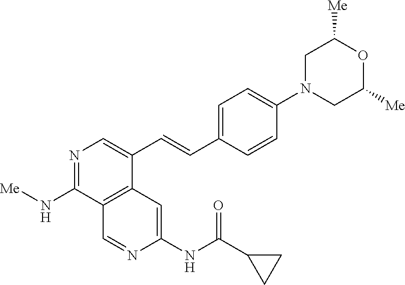 [SMILES: CNc1ncc(/C=C/c2ccc(N3C[C@@H](C)O[C@@H](C)C3)cc2)c2cc(NC(=O)C3CC3)ncc12]

To a solution of N-(5-bromo-8-(methylamino)-2,7-naphthyridin-3-yl)cyclopropanecarboxamide (86.0 mg; 0.268 mmol; 1.00 eq.) in DMF (2.5 mL) was added (2R,6S)-4-(4-ethenylphenyl)-2,6-dimethylmorpholine (70.0 mg; 0.322 mmol; 1.20 eq.), Pd(PPh₃)₂Cl₂ (18.8 mg; 0.027 mmol; 0.10 eq.) and Na₂CO₃ (57.0 mg; 0.538 mmol; 2.01 eq.). The mixture was stirred at 100° C. for 14 hours under nitrogen atmosphere. The desired product was detected via LCMS. The resulting mixture was diluted with EtOAc (50 mL) and washed with brine (3×10 mL). The organic layer was dried over anhydrous Na₂SO₄, filtered and concentrated under reduced pressure. The residue was purified by flash chromatography on silica gel column using 0-10% of MeOH in CH₂Cl₂ as eluent to provide 97.0 mg of the crude product that was repurified by Prep-Achiral-SFC (Column: DAICEL DCpak P4VP 3×25 cm, 5 μm; Mobile Phase A: CO₂, Mobile Phase B: MeOH (0.1% 2 M NH₃-MeOH); Flow rate: 60 mL/min; Gradient: isocratic 39.0% B; Column Temperature (° C.): 35; Back Pressure (bar): 100; Wave Length: 220 nm) to provide N-(5-((E)-4-((2R,6S)-2,6-dimethylmorpholino)styryl)-8-(methylamino)-2,7-naphthyridin-3-yl)cyclopropanecarboxamide as a yellow solid (38.9 mg, 31.1%). LCMS (ESI) m/z 458.2, [M+H]⁺. ¹H NMR (400 MHz, DMSO-d₆) δ 11.01 (s, 1H), 9.35 (s, 1H), 8.56 (s, 1H), 8.28 (s, 1H), 8.01-7.94 (m, 1H), 7.43 (d, J=8.4 Hz, 2H), 7.17 (d, J=16.0 Hz, 1H), 6.97 (d, J=8.4 Hz, 2H), 6.93 (d, J=16.0 Hz, 1H), 3.76-3.60 (m, 4H), 3.01 (d, J=4.4 Hz, 3H), 2.35-2.23 (m, 2H), 2.12-2.03 (m, 1H), 1.17 (d, J=6.2 Hz, 6H), 0.91-0.81 (m, 4H).

### Examples 787 and 788: Synthesis of (S,E)-N-(8-(methylamino)-5-(4-(2-methylmorpholino)styryl)-2,7-naphthyridin-3-yl)cyclopropanecarboxamide (Example 787) and (S)—N-(8-(methylamino)-5-(1-(4-(2-methylmorpholino)phenyl)vinyl)-2,7-naphthyridin-3-yl)cyclopropanecarboxamide (Example 788)

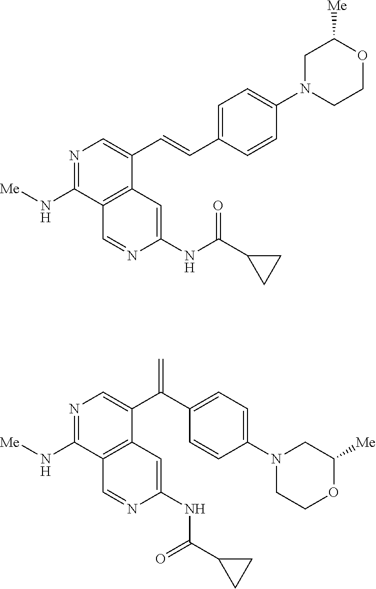 [SMILES: CNc1ncc(/C=C/c2ccc(N3CCO[C@@H](C)C3)cc2)c2cc(NC(=O)C3CC3)ncc12 ; C=C(c1ccc(N2CCO[C@@H](C)C2)cc1)c1cnc(NC)c2cnc(NC(=O)C3CC3)cc12]

### Step 1: (S)-4-(4-bromophenyl)-2-methylmorpholine

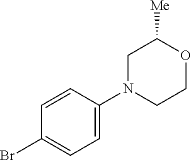 [SMILES: C[C@H]1CN(c2ccc(Br)cc2)CCO1]

To a solution of (S)-2-methylmorpholine (500.0 mg; 4.950 mmol; 1.00 eq.) in toluene (8 mL) were added 1,4-dibromobenzene (2.31 g; 9.871 mmol; 1.99 eq.), Pd₂(dba)₃ (512.3 mg; 0.495 mmol; 0.10 eq.), BINAP (615.7 mg; 0.990 mmol; 0.20 eq.) and sodium 2-methylpropan-2-olate (665.2 mg; 6.930 mmol; 1.40 eq.). The mixture was stirred at 80° C. for 14 hours under nitrogen atmosphere. The desired product was detected via LCMS. The reaction was concentrated under pressure and purified by flash chromatography on silica gel column using 75-85% of EtOAc in petroleum ether as eluent to provide (S)-4-(4-bromophenyl)-2-methylmorpholine as a white solid (757.4 mg, 60.0%). LCMS (ESI) m/z 256.0, [M+H]⁺.

### Step 2: (S)-2-methyl-4-(4-vinylphenyl)morpholine

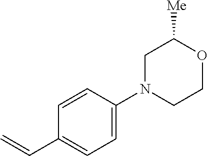

To a stirred solution of (S)-4-(4-bromophenyl)-2-methylmorpholine (100.0 mg; 0.390 mmol; 1.00 eq.) in a mixture solvent of 1,4-dioxane/water (5:1, 1.2 mL) were added 4,4,5,5-tetramethyl-2-vinyl-1,3,2-dioxaborolane (66.0 mg; 0.429 mmol; 1.10 eq.), S-Phos (16.0 mg; 0.039 mmol; 0.10 eq.), K₃PO₄ (248.4 mg; 1.170 mmol; 3.00 eq.) and Pd(OAc)₂ (4.4 mg; 0.020 mmol; 0.05 eq.). The mixture was stirred at 100° C. for 14 hours under nitrogen atmosphere. The desired product was detected via LCMS. The reaction was concentrated under pressure. The residue was purified by flash chromatography on silica gel column using 80-90% of EtOAc in petroleum ether as eluent to provide (S)-2-methyl-4-(4-vinylphenyl)morpholine as an off-white solid (55.0 mg, 60.1%). LCMS (ESI) m/z 204.1, [M+H]⁺.

### Step 3: (S,E)-N-(8-(methylamino)-5-(4-(2-methylmorpholino)styryl)-2,7-naphthyridin-3-yl) cyclopropanecarboxamide and (S)—N-(8-(methylamino)-5-(1-(4-(2-methylmorpholino)phenyl)vinyl)-2,7-naphthyridin-3-yl)cyclopropanecarboxamide

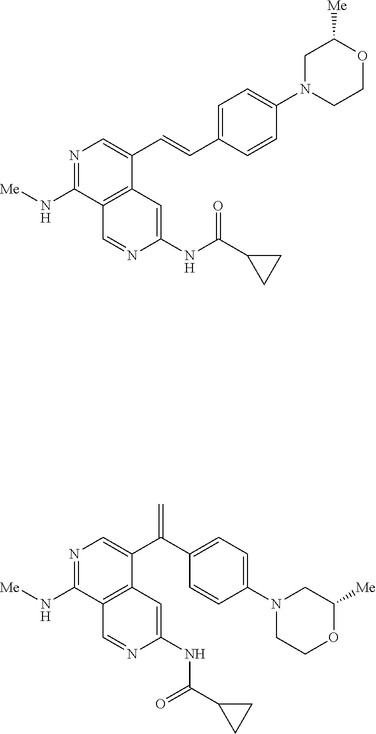 [SMILES: CNc1ncc(/C=C/c2ccc(N3CCO[C@@H](C)C3)cc2)c2cc(NC(=O)C3CC3)ncc12 ; C=C(c1ccc(N2CCO[C@@H](C)C2)cc1)c1cnc(NC)c2cnc(NC(=O)C3CC3)cc12]

To a solution of N-(5-bromo-8-(methylamino)-2,7-naphthyridin-3-yl)cyclopropanecarboxamide (73.0 mg; 0.227 mmol; 1.00 eq.) in DMF (2 mL) were added (S)-2-methyl-4-(4-vinylphenyl)morpholine (55.0 mg; 0.271 mmol; 1.19 eq.), Pd(PPh₃)₂Cl₂ (16.0 mg; 0.023 mmol; 0.10 eq.) and Na₂CO₃ (48.0 mg; 0.453 mmol; 1.99 eq.). The mixture was stirred at 100° C.; for 7 hours under nitrogen atmosphere. The desired product was detected via LCMS. The reaction was concentrated under pressure. The resulting mixture was diluted with EtOAc (50 mL) and washed with brine (3×10 mL). The organic layer was dried over anhydrous Na₂SO₄, filtered and concentrated under reduced pressure. The residue was purified by flash chromatography on silica gel column using 0-10% of MeOH in CH₂Cl₂ as eluent to provide the crude product that was repurified by Prep-Achiral-SFC (Column: DAICEL DCpak P4VP 3×25 cm, 5 urn; Mobile Phase A: CO₂, Mobile Phase B: MeOH (0.1% 2 M NH₃-MeOH); Flow rate: 60 mL/min, Gradient: isocratic 50.0% B; Column Temperature (° C.): 35; Back Pressure(bar): 100; Wave Length: 254 nm) to provide (S,E)-N-(8-(methylamino)-5-(4-(2-methylmorpholino)styryl)-2,7-naphthyridin-3-yl)cyclopropanecarboxamide as a yellow solid (24.6 mg, 24.3%) and (S)—N-(8-(methylamino)-5-(1-(4-(2-methylmorpholino)phenyl)vinyl)-2,7-naphthyridin-3-yl)cyclopropanecarboxamide as an off-white solid (5.3 mg, 5.1%). LCMS (ESI) m/z 444.2, [M+H]⁺.

HNMR for example 787: NMR (400 MHz, DMSO-d₆) δ 11.01 (s, 9.34 (s, 1H), 8.56 (s, 1H), 8.29 (s, 1H), 7.98-7.92 (m, 1H), 7.43 (d, J=8.4 Hz, 2H), 7.17 (d, J=16.0 Hz, 1H), 6.97 (d, J=8.4 Hz, 2H), 6.96 (d, J=16.0 Hz, 1H), 3.96-3.90 (m, la), 3.70-3.50 (m, 4H), 3.00 (d, =4.4 Hz, 31:1), 2.70-2.50 (m, 1H), 2.38-2.32 (m, 1H), 2.07-2.04 (m, 1H), 1.17 (d, J=6.0 Hz, 3H), 0.91-0.79 (m, 4H).

### Examples 789, 790, and 791: Synthesis of (E)-N-(5-(2-(2-(methoxymethyl)-2,3-dihydrobenzo[b][1,4]dioxin-6-yl)vinyl)-8-(methylamino)-2,7-naphthyridin-3-yl)cyclopropanecarboxamide (Example 789) and (S,E)-N-(5-(2-(2-(methoxymethyl)-2,3-dihydrobenzo[b][1,4]dioxin-6-yl)vinyl)-8-(methylamino)-2,7-naphthyridin-3-yl)cyclopropanecarboxamide (Example 790) and (R,E)-N-(5-(2-(2-(methoxymethyl)-2,3-dihydrobenzo[b][1,4]dioxin-6-yl)vinyl)-8-(methylamino)-2,7-naphthyridin-3-yl)cyclopropanecarboxamide (Example 791)

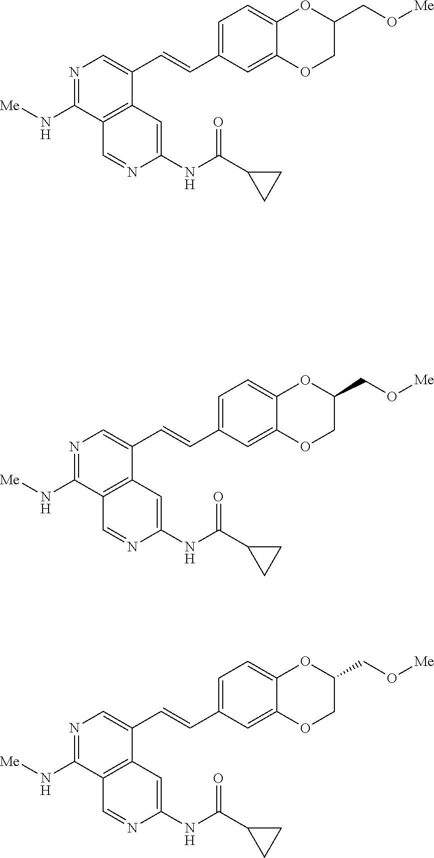 [SMILES: CNc1ncc(/C=C/c2ccc3c(c2)OCC(COC)O3)c2cc(NC(=O)C3CC3)ncc12 ; CNc1ncc(/C=C/c2ccc3c(c2)OC[C@H](COC)O3)c2cc(NC(=O)C3CC3)ncc12 ; CNc1ncc(/C=C/c2ccc3c(c2)OC[C@@H](COC)O3)c2cc(NC(=O)C3CC3)ncc12]

### Step 1: 2-(methoxymethyl)-6-vinyl-2,3-dihydrobenzo[b][1,4]dioxine

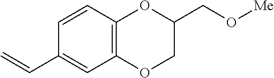

A solution of 6-bromo-2-(methoxymethyl)-2,3-dihydrobenzo[b][1,4]dioxine (Example 69, step 1) (275.0 mg; 1.061 mmol; 1.00 eq.), 4,4,5,5-tetramethyl-2-vinyl-1,3,2-dioxaborolane (179.8 mg; 1.167 mmol; 1.10 eq.), S-Phos (43.6 mg; 0.106 mmol; 0.10 eq.), K₃PO₄ (675.9 mg; 3.183 mmol; 3.00 eq.) and Pd(AcO)₂ (11.9 mg; 0.053 mmol; 0.05 eq.) in a mixture solvent of 1,4-dioxane/water (5:1, 6 mL) was stirred at 100° C. for 4 hours under nitrogen atmosphere. The desired product was detected via LCMS. The reaction was concentrated under pressure and purified by flash chromatography on silica gel column using 0-30% of EtOAc in petroleum ether as eluent to provide 2-(methoxymethyl)-6-vinyl-2,3-dihydrobenzo[b][1,4]dioxine as a light yellow oil (164.4 mg, 65.1%). LCMS (ESI) m/z 207.1, [M+H]⁺.

### Step 2: (E) N-(5-(2-(2-(methoxymethyl)-2,3-dihydrobenzo[b][1,4]dioxin-6-yl)vinyl)-8-(methylamino)-2,7-naphthyridin-3-yl)cyclopropanecarboxamide

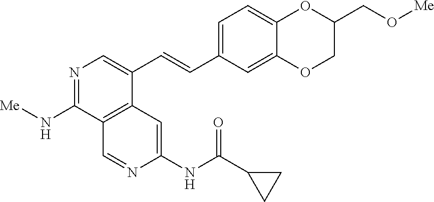 [SMILES: CNc1ncc(/C=C/c2ccc3c(c2)OCC(COC)O3)c2cc(NC(=O)C3CC3)ncc12]

A solution of 2-(methoxymethyl)-6-vinyl-2,3-dihydrobenzo[b][1,4]dioxine (96.3 mg; 0.467 mmol; 1.50 eq.), N-(5-bromo-8-(methylamino)-2,7-naphthyridin-3-yl)cyclopropanecarboxamide (100.0 mg; 0.311 mmol; 1.00 eq.), Pd(PPh₃)₂Cl₂ (21.8 mg; 0.031 mmol; 0.10 eq.) and Na₂CO₃ (66.0 mg; 0.622 mmol; 2.00 eq.) in DMF (4.0 mL) was stirred at 100° C. for 14 hours under nitrogen atmosphere. The desired product was detected via LCMS. The resulting mixture was diluted with EtOAc (50 mL) and washed with brine (3×5 mL). The organic layer was dried over anhydrous Na₂SO₄, filtered, concentrated under vacuum and purified by flash chromatography on silica gel column using 10-20% of MeOH in CH₂Cl₂ to provide the crude product that was repurified by Prep-Achiral-SFC (Column: DAICEL DCpak P4VP 3×25 cm, 5 μm; Mobile Phase A: CO₂, Mobile Phase B: MeOH (0.1% 2 M NH₃-MeOH); Flow rate: 60 mL/min; Gradient: isocratic 47% B; Column Temperature (° C.): 35; Back Pressure(bar): 100; Wave Length: 254 nm) to provide (E)-N-(5-(2-(2-(methoxymethyl)-2,3-dihydrobenzo[b][1,4]dioxin-6-yl)vinyl)-8-(methylamino)-2,7-naphthyridin-3-yl)cyclopropanecarboxamide as a yellow solid (50.0 mg, 31.2%). LCMS (ESI) m/z 447.2, [M+H]⁺. ¹H NMR (400 MHz, DMSO-d₆) δ 1.1.02 (s, 1H), 9.35 (s, 1H), 8.55 (s, 1H), 8.29 (s, 1H), 8.02-7.96 (m, 1H), 7.20 (d, J=16.4 Hz, 1H), 7.12-7.00 (m, 2H), 6.95 (d, J 16.4 Hz, 1H), 6.90 (d, J=8.4 Hz, 1H), 4.42-4.30 (m, 2H), 4.10-4.00 (m, 1H), 3.67-3.55 (m, 2H), 3.33 (s, 3H), 3.00 (d, J=4.4 Hz, 3H), 2.12-2.02 (m, 1H), 0.93-0.78 (m, 4H).

### Step 3: (S,E)-N-(5-(2-(2-(methoxymethyl)-2,3-dihydrobenzo[b][1,4]dioxin-6-yl)vinyl)-8-(methylamino)-2,7-naphthyridin-3-yl)cydopropanecarboxamide and (R,E)-N-(5-(2-(2-(methoxymethyl)-2,3-dihydrobenzo[b][1,4]dioxin-6-yl)vinyl)-8-(methylamino)-2,7-naphthyridin-3-yl) cyclopropanecarboxamide

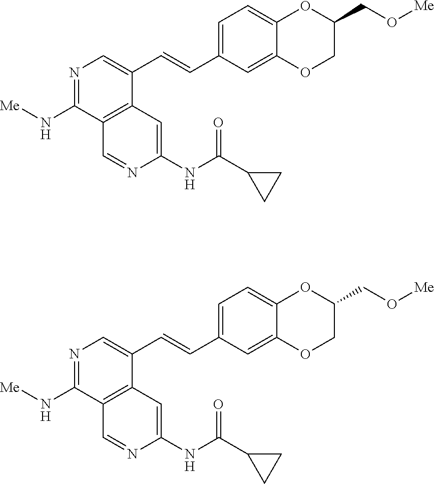 [SMILES: CNc1ncc(/C=C/c2ccc3c(c2)OC[C@H](COC)O3)c2cc(NC(=O)C3CC3)ncc12 ; CNc1ncc(/C=C/c2ccc3c(c2)OC[C@@H](COC)O3)c2cc(NC(=O)C3CC3)ncc12]

(E)-N-(5-(2-(2-(methoxymethyl)-2,3-di hydrobenzo[b][1,4]dioxin-6-yl)vinyl)-8-(methylamino)-2,7-naphthyridin-3-yl)cyclopropanecarboxamide was separated by Chiral-HPLC (Column: Lux 5 um Cellulose-4, 2.12×25 cm, 5 μm; Mobile Phase A: Hex (0.5% 2 M NH₃-MeOH), Mobile Phase B: MeOH:EtOH=1:1; Flow rate: 20 mL/min; Gradient: 25% B to 25% B in 15 min; Wave Length: 220/254 nm; R_{T1}(min): 12.17; R_{T2}(min): 14.51) to provide (S,E)-N-(5-(2-(2-(methoxymethyl)-2,3-dihydrobenzo[b][1,4]dioxin-6-yl)vinyl)-8-(methylamino)-2,7-naphthyridin-3-yl)cyclopropanecarboxamide (Example 790, the faster peak) as a yellow solid and (R,E)-N-(5-(2-(2-(methoxymethyl)-2,3-dihydrobenzo[b][1,4]dioxin-6-yl)vinyl)-8-(methylamino)-2,7-naphthyridin-3-yl)cyclopropanecarboxamide (Example 791, the slower peak) as a yellow solid. The two configurations are arbitrarily assigned. LCMS (ESI) m/z 447.2, [M+H]⁺. HNMR for Example 790: ¹H NMR (400 MHz, DMSO-d₆) δ 11.02 (s, 1H), 9.35 (s, 1H), 8.55 (s, 1H), 8.29 (s, 1H), 8.02-7.96 (m, 1H), 7.20 (d, J=16.4 Hz, 1H), 7.12-7.00 (m, 2H), 6.95 (d, J=16.4 Hz, 1H), 6.90 (d, J=8.4 Hz, 1H), 4.42-4.30 (m, 2H), 4.10-4.00 (m, 1H), 3.67-3.55 (m, 2H), 3.33 (s, 3H), 3.00 (d, J=4.4 Hz, 3H), 2.12-2.02 (m, 1H), 0.93-0.78 (m, 4H). HNMR for Example 791: ¹H NMR (400 MHz, DMSO-d₆) δ 11.02 (s, 1H), 9.35 (s, 1H), 8.55 (s, 1H), 8.29 (s, 1H), 8.02-7.96 (m, 1H), 7.20 (d, J=16.4 Hz, 1H), 7.12-7.00 (m, 2H), 6.95 (d, J=16.4 Hz, 1H), 6.90 (d, J=8.4 Hz, 1H), 4.42-4.30 (m, 2H), 4.1.0-4.00 (m, 1H), 3.67-3.55 (m, 2H), 3.33 (s, 3H), 3.00 (d, J=4.4 Hz, 3H), 2.12-2.02 (m, 1H), 0.93-0.78 (m, 4H).

### Examples 792 and 793: Synthesis of (E)-N-(8-(methylamino)-5-(4-morpholinostyryl)-2,7-naphthyridin-3-yl)cyclopropanecarboxamide (Example 792) and N-(8-(methylamino)-5-(1-(4-morpholinophenyl)vinyl)-2,7-naphthyridin-3-yl)cyclopropanecarboxamide (Example 793)

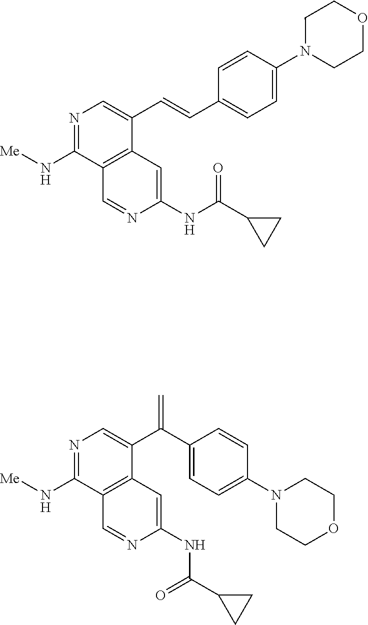 [SMILES: CNc1ncc(/C=C/c2ccc(N3CCOCC3)cc2)c2cc(NC(=O)C3CC3)ncc12 ; C=C(c1ccc(N2CCOCC2)cc1)c1cnc(NC)c2cnc(NC(=O)C3CC3)cc12]

### Step 1: 4-(4-vinylphenyl)morpholine

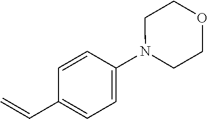

A solution of 4-(4-bromophenyl)morpholine (200.0 mg; 0.826 mmol; 1.00 eq.), 4,4,5,5-tetramethyl-2-vinyl-1,3,2-dioxaborolane (139.9 mg; 0.909 mmol; 1.10 eq.), S-Phos (33.9 mg; 0.083 mmol; 0.10 eq.), K₃PO₄ (526.0 mg; 2.478 mmol; 3.00 eq.), and Pd(AcO)₂ (9.2 mg; 0.041 mmo; 0.05 eq.) in a mixture solvent of 1,4-dioxane/water (5:1, 6 mL) was stirred at 100° C. for 1 hour under nitrogen atmosphere. The desired product was detected via LCMS. The solvent was concentrated under vacuum and purified by flash chromatography on silica gel column using 5-10% of EtOAc in petroleum ether as eluent to provide 4-(4-vinylphenyl)morpholine as a light yellow oil (124.0 mg, 78.1%). LCMS (ESI) m/z 190.1, [M+H]⁺.

### Step 2: (E)-N-(8-(methylamino)-5-(4-morpholinostyryl)-2,7-naphthyridin-3-yl)cyclopropanecarboxamide and N-(8-(methylamino)-5-(1-(4-morpholinophenyl)vinyl)-2,7-naphthyridin-3-yl)cyclopropanecarboxamide

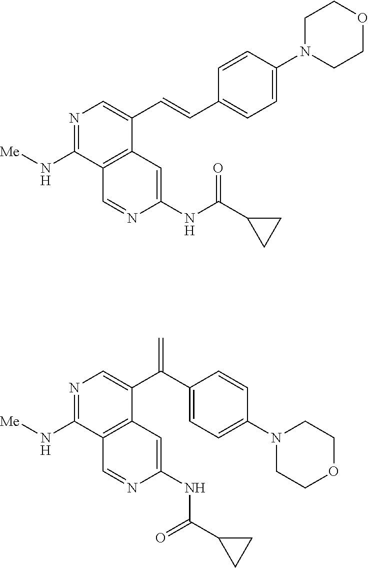 [SMILES: CNc1ncc(/C=C/c2ccc(N3CCOCC3)cc2)c2cc(NC(=O)C3CC3)ncc12 ; C=C(c1ccc(N2CCOCC2)cc1)c1cnc(NC)c2cnc(NC(=O)C3CC3)cc12]

A solution of 4-(4-vinylphenyl)morpholine (88.3 mg; 0.467 mmol; 1.50 eq.), N-(5-bromo-8-(methylamino)-2,7-naphthyridin-3-yl)cyclopropanecarboxamide (100.0 mg; 0.311 mmol; 1.00 eq.), Pd(PPh₃)₂Cl₂ (21.8 mg; 0.031 mmol; 0.1.0 eq.) and Na₂CO: (66.0 mg; 0.622 mmol; 2.00 eq.) in DMF (4 mL) was stirred at 100° C. for 14 hours under nitrogen atmosphere. The desired product was detected via LCMS. The resulting mixture was diluted with EtOAc (50 mL) and washed with brine (3×5 mL). The organic layer was dried over anhydrous Na₂SO₄. After filtration, the filtrate was concentrated under reduced pressure. The residue was purified by flash chromatography on silica gel column using 10-20% of MeOH in CH₂Cl₂ as eluent to provide the crude product. The crude product was purified by Prep-achiral-SFC (YMC-Actus Triart Diol-HILIC 3×25 cm, 5 μm; Mobile Phase A: CO₂, Mobile Phase B: MeOH (0.1% 2 M NH; MeOH); Flow rate: 75 mL/min; Gradient: isocratic 25.0% B; Column Temperature (° C.): 35; Back Pressure (bar): 100; Wave Length: 220 nm) to provide (E)-N-(8-(methylamino)-5-(4-morpholinostyryl)-2,7-naphthyridin-3-yl)cyclopropanecarboxamide (Example 792) as a yellow solid (52.5 mg, 39.4%) and N-(8-(methylamino)-5-(1-(4-morpholinophenyl)vinyl)-2,7-naphthyridin-3-yl)cyclopropanecarboxamide (Example 793) as an off-white solid (15.3 mg, 1.1.5%). LCMS (ESI) m/z 430.1, [M+H]⁺.

HNMR for example 792: ¹H NMR (400 MHz, DMSO-d₆) δ 11.02 (s, 1H), 9.34 (s, 1H), 8.56 (s, 1H), 8.29 (s, 1H), 7.99-7.95 (m, 1H), 7.44 (d, J=8.4 Hz, 2H), 7.18 (d, J=1.6.0 Hz, 1H), 7.00-6.95 (m, 3H), 3.80-3.70 (m, 4H), 3.18-3.10 (m, 4H), 3.00 (d, J=4.4 Hz, 3H), 2.13-2.01 (m, 1H), 0.92-0.80 (m, 4H).

### Example 794: Synthesis of (E) N-(5-(4-(2-cyanoethoxy)styryl)-8-(methylamino)-2,7-naphthyridin-3-yl)cyclopropanecarboxamide

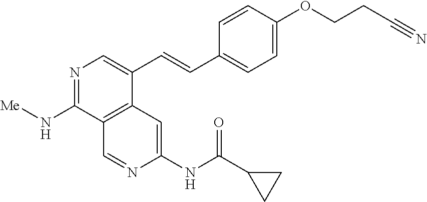 [SMILES: CNc1ncc(/C=C/c2ccc(OCCC#N)cc2)c2cc(NC(=O)C3CC3)ncc12]

### Step 1: 6-chloro-N-methyl-2,7-naphthyridin-1-amine

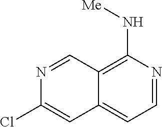 [SMILES: CNc1nccc2cc(Cl)ncc12]

To a solution of 6-chloro-2,7-naphthyridin-1(2H)-one (1.00 g; 5.530 mmol; 1.00 eq.) and PyBOP (5.70 g; 10.950 mmol; 2.00 eq.) in DMA (15 mL) were added methanamine hydrochloride (1.11 g; 16.440 mmol; 3.00 eq.) and DIPEA (3.60 g; 27.850 mmol; 5.00 eq.). The mixture was stirred at 80° C. for 16 hours under nitrogen atmosphere. The desired product was detected via LCMS. The resulting mixture was diluted with EtOAc (150 mL) and washed with brine (5×10 mL). The organic layer was dried over anhydrous Na₂SO₄, filtered, and concentrated under vacuum. The residue was purified by flash chromatography on silica gel column using 10-40% of EtOAc in CH₂Cl₂ as eluent to provide a crude product. The crude product was purified by flash chromatography on pre-packed C18 column using 20-50% of MeCN in water (10 mmol/L NH₄HCO₃) as eluent to provide 6-chloro-N-methyl-2,7-naphthyridin-1-amine as a off-white solid (676.0 mg, 63.7%). LCMS (ESI) m/z 194.0, [M+H]⁺.

### Step 2: 6-chloro-4-iodo-N-methyl-2,7-naphthyridin-1-amine

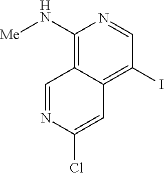 [SMILES: CNc1ncc(I)c2cc(Cl)ncc12]

To a solution of 6-chloro-N-methyl-2,7-naphthyridin-1-amine (612.0 mg; 3.160 mmol; 1.00 eq.) in DMF (12 mL) was added MS (1.07 g; 4.750 mmol; 1.50 eq.) in portions at 0° C. The mixture was stirred at room temperature for 1 hour. The desired product was detected via LCMS. The resulting mixture was diluted with EtOAc (150 mL) and washed with brine (5×10 mL). The organic layer was dried over anhydrous Na₂SO₄, filtered, and concentrated under vacuum. The residue was slurred with CH₂Cl₂ (15 mL) to precipitate solids. The precipitated solids were collected by filtration and washed with CH₂Cl₂ to afford 6-chloro-4-iodo-N-methyl-2,7-naphthyridin-1-amine as a yellow solid (983.0 mg, 97.2%). LCMS (ESI) m/z 319.9, [M+H]⁺.

### Step 3: (E)-N-(8-(methylamino)-5-(2-(tributylstannyl)vinyl)-2,7-naphthyridin-3-yl)cyclopropanecarboxamide

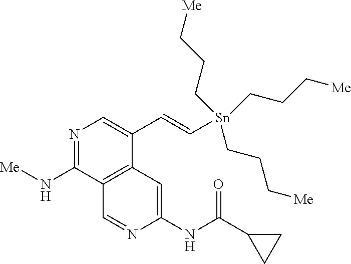 [SMILES: CCC[CH2][Sn](/[CH]=C/c1cnc(NC)c2cnc(NC(=O)C3CC3)cc12)([CH2]CCC)[CH2]CCC]

A solution of N-(5-bromo-8-(methylamino)-2,7-naphthyridin-3-yl)cyclopropanecarboxamide (600.0 mg; 1.869 mmol; 1.00 eq.), (E)-1,2-bis(tributylstannyl)ethene (1.20 g; 1.980 mmol; 1.06 eq.) and Pd(PPh₃)₄ (47.2 mg; 0.042 mmol; 0.02 eq.) in toluene (8 mL) was stirred at 110° C. for 2 hours under nitrogen atmosphere. The desired product was detected via LCMS. The reaction system was used directly for the next step without further purification. LCMS (ESI) m/z 559.2, [M+H]⁺.

### Step 4: (E)-N-(5-(4-(2-cyanoethoxy)styryl)-8-(methylamino)-2,7-naphthyridin-3-yl)cyclopropanecarboxamide

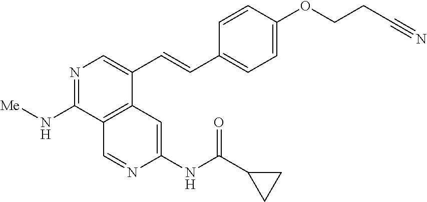 [SMILES: CNc1ncc(/C=C/c2ccc(OCCC#N)cc2)c2cc(NC(=O)C3CC3)ncc12]

To a solution of 3-(4-bromophenoxy)propanenitrile (462.6 mg; 2.046 mmol; 1.00 eq.) and Pd(PPh₃)₄ (47.2 mg; 0.042 mmol; 0.02 eq.) in toluene (8 mL) was added (E)-N-(8-(methyl amino)-5-(2-(tributyl stannyl)vinyl)-2,7-naphthyridin-3-yl)cyclopropanecarboxamide. The mixture was stirred at 110° C. for 3 hours under nitrogen atmosphere. The desired product was detected via LCMS. The resulting mixture was concentrated under reduced pressure. The residue was purified by flash chromatography on silica gel column using 80-90% of EtOAc in petroleum ether as eluent to provide the crude product. The product was re-crystallized with a mixture solvent of CH₂Cl₂/MeOH (10:1, 10 mL) to provide (E)-N-(5-(4-(2-cyanoethoxy)styryl)-8-(methylamino)-2,7-naphthyridin-3-yl)cyclopropanecarboxamide as a yellow solid (155.9 mg, 52.1%). LCMS (ESI) m/z 414.2, [M+H]⁺. 1H NMR (400 MHz, DMSO-d₆) δ 11.03 (s, 1H), 9.35 (s, 1H), 8.56 (s, 1H), 8.30 (s, 1H), 8.10-7.98 (m, 1H), 7.53 (d, J=8.4 Hz, 2H), 7.24 (d, J=16.4 Hz, 1H), 7.06 (d, J=16.4 Hz, 1H), 7.01 (d, J=8.4 Hz, 2H), 4.22 (t, J=5.6 Hz, 2H), 3.03 (t, J=5.6 Hz, 2H), 3.00 (d, J=4.4 Hz, 3H), 2.11-2.00 (m, 1H), 0.92-0.79 (m, 4H).

### Example 795: Synthesis of (E)-4-(2-(1 (methylamino)-6-(pyridin-2-ylamino)-2,7-naphthyridin-4-yl)vinyl)phenol

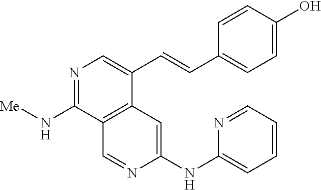 [SMILES: CNc1ncc(/C=C/c2ccc(O)cc2)c2cc(Nc3ccccn3)ncc12]

### Step 1: (E)-3-(4-(2-(tributylstannyl)vinyl)phenoxy)propanenitrile

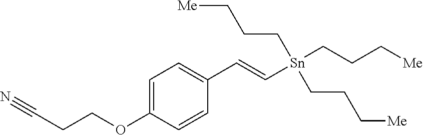 [SMILES: CCC[CH2][Sn](/[CH]=C/c1ccc(OCCC#N)cc1)([CH2]CCC)[CH2]CCC]

A solution of 3-(4-bromophenoxy)propanenitrile (98.7 mg; 0.437 mmol; 1.00 eq.), (E)-1,2-bis(tributylstannyl)ethene (277.8 mg; 0.458 mmol; 1.05 eq.) and Pd(PPh₃)₄ (10.1 mg; 0.009 mmol; 0.02 eq.) in toluene (2 mL) was stirred at 1.10° C. for 2 hours under nitrogen atmosphere. The reaction system was used directly for the next step without further purification.

### Step 2: (E)-3-(4-(2-(6-chloro-1-(methylamino)-2,7-naphthyridin-4-yl)vinyl)phenoxy)propanenitrile

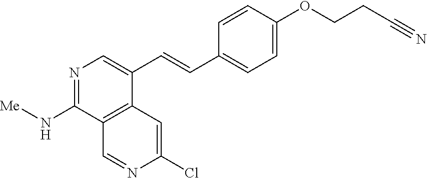 [SMILES: CNc1ncc(/C=C/c2ccc(OCCC#N)cc2)c2cc(Cl)ncc12]

To a solution of 6-chloro-4-iodo-N-methyl-2,7-naphthyridin-1-amine (141.1 mg; 0.442 mmol; 1.00 eq.) and Pd(PPh₃)₄ (10.2 mg; 0.009 mmol; 0.02 eq.) in toluene (2 mL ) was added (E)-3-(4-(2-(tributylstannyl)vinyl)phenoxy)propanenitrile. The mixture was stirred at 90° C. for 2 hours under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide 281.6 mg of the crude product as a yellow solid. The crude product was repurified by flash chromatography on pre-packed C18 column using 40-60% of MeCN in water (0.05% TFA) as eluent to provide (E)-3-(4-(2-(6-chloro-1-(methylamino)-2,7-naphthyridin-4-yl)vinyl)phenoxy)propanenitrile as a yellow solid (52.0 mg, 32.2%). LCMS (ESI) m/z 365.1, [M+H]⁺.

### Step 3: (E)-4-(2-(1-(methylamino)-6-(pyridin-2-ylamino)-2,7-naphthyridin-4-yl)vinyl)phenol

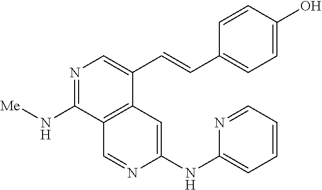 [SMILES: CNc1ncc(/C=C/c2ccc(O)cc2)c2cc(Nc3ccccn3)ncc12]

A solution of (E)-3-(4-(2-(6-chloro-1-(methylamino)-2,7-naphthyridin-4-yl)vinyl)phenoxy)propanenitrile (30.0 mg; 0.082 mmol; 1.00 eq.), pyridin-2-amine (23.2 mg; 0.247 mmol; 3.00 eq.), Pd₂(dba)₃(7.5 mg; 0.008 mmol; 0.10 eq.), XantPhos (9.5 mg; 0.016 mmol; 0.20 eq.) and Cs₂CO₃ (53.6 mg; 0.165 mmol; 2.00 eq.) in 1,4-dioxane (2 mL) was stirred at 130° C. for overnight under nitrogen atmosphere. The desired product was detected via LCMS. The resulting mixture was concentrated under reduced pressure. The residue was purified by flash chromatography on silica gel column using 0-10% of MeOH: in CH₂Cl₂ as eluent to provide the crude product. The crude product was purified by flash chromatography on pre-packed C18 column using 60-80% of MeCN in water (10 mmol/L NH₄HCO₃) as eluent to provide (E)-4-(2-(1-(methylamino)-6-(pyridin-2-ylamino)-2,7-naphthyridin-4-yl)vinyl)phenol as a yellow solid (4.8 mg, 15.2%). LCMS (ESI) m/z 370.2, [M+H]⁺.

### Example 796: Synthesis of N-(8-(methylamino)-5-(6-morpholinopyrazolo[1,5-a]pyridin-2-yl)-2,7-naphthyridin-3-yl)cyclopropanecarboxamide

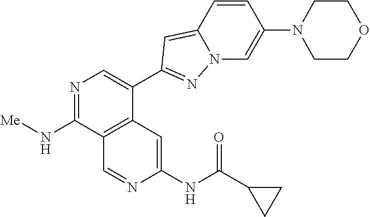 [SMILES: CNc1ncc(-c2cc3ccc(N4CCOCC4)cn3n2)c2cc(NC(=O)C3CC3)ncc12]

### Step 1: 6-bromopyrazolo[1,5-a]pyridin-2-ol

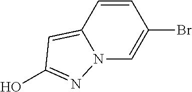 [SMILES: Oc1cc2ccc(Br)cn2n1]

A solution of methyl 2-(5-bromopyridin-2-yl)acetate (1.21 g; 4.342 mmol; 1.00 eq.) and 0-(mesitylsulfonyl)hydroxylamine (3.71 g; 17.182 mmol; 4.00 eq.) in CH₂Cl₂ (10 mL) was stirred at room temperature for 14 hours under nitrogen atmosphere. The desired product was detected via LCMS. The resulting mixture was concentrated under reduced pressure. The residue was purified by flash chromatography on silica gel column using 5-8% of MeOH in CH₂Cl₂ as eluent to provide 6-bromopyrazolo[1,5-a]pyridin-2-ol as a red solid (500.2 mg, 54.0%). LCMS (ESI) m/z 213.0, [M+H]⁺.

### Step 2: 6-bromo-2-(m ethoxymethoxy)pyrazolo[1,5-a]pyridine

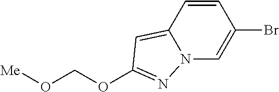 [SMILES: COCOc1cc2ccc(Br)cn2n1]

To a stirred solution of 6-bromopyrazolo[1,5-a]pyridin-2-ol (250.2 mg; 1.174 mmol; 1.00 eq.) and DIEA (380.5 mg; 2.941 mmol; 2.50 eq.) in CH₂Cl₂ (8 mL) was added bromo(methoxy)methane (292.3 mg; 2.334 mmol; 2.00 eq.) in portions at 0° C. under nitrogen atmospheres. The resulting solution was stirred at room temperature for 0.5 h. The desired product was detected via LCMS. The resulting mixture was concentrated under reduced pressure and purified by flash chromatography, on silica gel column, using 10-30% of EtOAc in petroleum ether as eluent to provide 6-bromo-2-(methoxymethoxy)pyrazolo[1,5-]pyridine as a colorless oil (200.2 mg, 66.2%). LCMS (ESI) m/z 257.0, [M+H]⁺.

### Step 3: 4-(2-(methoxymethoxy)pyrazolo[1,5-a]pyridin-6-yl)morpholine

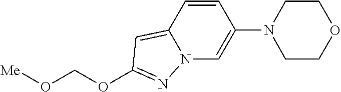 [SMILES: COCOc1cc2ccc(N3CCOCC3)cn2n1]

To a stirred solution of 6-bromo-2-(methoxymethoxy)pyrazolo[1,5-a]pyridine (200.2 mg; 0.771 mmol; 1.00 eq.) and morpholine (68.1 mg; 0.784 mmol; 1.00 eq.) in 1,4-dioxane (3 mL) were added Pd-PEPPSI-IHeptCl 3-chloropyridine (76.2 mg; 0.078 mmol; 0.10 eq.) and Cs₂CO₃ (509.2 mg; 1.562 mmol; 2.00 eq.) under nitrogen atmosphere. The resulting mixture was stirred at 100° C. for 3 hours. The desired product was detected via LCMS. The resulting mixture was concentrated under reduced pressure and purified by flash chromatography on silica gel column using 5-8% of MeOH in CH₂Cl₂ as eluent to provide 4-[2-(methoxymethoxy)pyrazolo[1,5-a]pyridin-6-yl]morpholine as a green oil (130.2 mg, 63.4%). LCMS (ESI) m/z 264.1, [M+H]⁺.

### Step 4: 6-morpholinopyrazolo[1,5-a]pyridin-2-ol

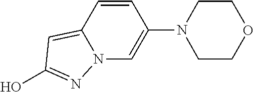 [SMILES: Oc1cc2ccc(N3CCOCC3)cn2n1]

To a stirred solution of 4-(2-(methoxymethoxy)pyrazolo[1,5-a]pyridin-6-yl)morpholine (90.1 mg; 0.341 mmol; 1.00 eq.) in methanol (1 mL) was added HCl (4M in 1,4-dioxane, 1 mL). The resulting solution was stirred at room temperature for 1 hour. The desired product was detected via LCMS. The resulting mixture was concentrated under reduced pressure. The residue was purified by flash chromatography on silica gel column using 5-8% of MeOH in CH₂Cl₂ as eluent to provide 6-(morpholin-4-yl)pyrazolo[1,5-a]pyridin-2-ol as a red solid (60.4 mg, 80.0%). LCMS (ESI) m/z 220.1, [M+H]⁺.

### Step 5: 6-morpholinopyrazolo[1,5-a]pyridin-2-yl trifluoromethanesulfonate

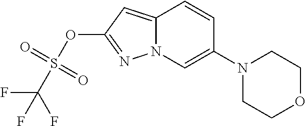 [SMILES: O=S(=O)(Oc1cc2ccc(N3CCOCC3)cn2n1)C(F)(F)F]

To a stirred solution of 6-(morpholin-4-yl)pyrazolo[1.54]pyridin-2-ol (60.4 mg; 0.272 mmol; 1.00 eq.) and DIEA (106.1 mg; 0.812 mmol; 2.50 eq.) in CH₂Cl₂ (2 mL) was added Tf₂O (115.8 mg; 0.411 mmol; 1.50 eq.) dropwise at 0° C. The resulting solution was stirred at room temperature for 1 hour under nitrogen atmosphere. The desired product was detected via LCMS. The mixture was diluted with CH₂Cl₂ (20 mL) and washed with water (2×10 mL). The organic layer was dried over anhydrous Na₇SO₄ and concentrated under reduced pressure to provide 6-(morpholin-4-yl)pyrazolo[1,5-c]pyridin-2-yl trifluoromethanesulfonate as a green oil (80.3 mg, crude). The crude product was used in the next step directly without further purification. LCMS (ESI) m/z 352.1, [M+H]⁺.

### Step 6: N-(8-(methylamino)-5-(6-morpholinopyrazolo[1,5-a]pyridin-2-yl)-2,7-naphthyridin-3-yl)cyclopropanecarboxamide

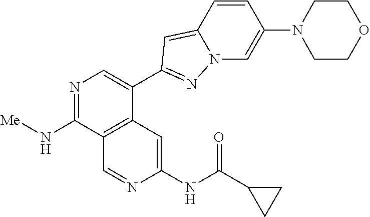 [SMILES: CNc1ncc(-c2cc3ccc(N4CCOCC4)cn3n2)c2cc(NC(=O)C3CC3)ncc12]

To a solution of 6-(morpholin-4-yl)pyrazolo[1,5-a]pyridin-2-yltrifluoromethanesulfonate (80.3 mg; 0.221 mmol, 1.00 eq.) and N-[8-(methylamino)-5-(4,4,5,5-tetramethyl-1,3,2-dioxaborolan-2-yl)-2,7-naphthyridin-3-yl]cyclopropanecarboxamide (92.2 mg; 0.253 mmol; 1.10 eq.) in a mixture solvent of 1,4-dioxane/water (5:1, 1 mL) were added XPhos Pd G₃ (19.2 mg; 0.023 mmol; 0.10 eq.), XPhos (21.7 mg; 0.042 mmol; 0.20 eq.) and K₃PO₄ (145.5 mg; 0.684 mmol; 3.00 eq.) under nitrogen atmosphere. The resulting mixture was stirred at 110° C. for 1 hour. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide the crude product. The crude product was purified by flash chromatography on pre-packed C18 column using 20-60% of MeCN in water (10 mmol/L NH₄HCO₃) to provide N-(8-(methylamino)-5-(6-morpholinopyrazolo[1,5-a]pyridin-2-yl)-2,7-naphthyridin-3-yl)cyclopropanecarboxamide as a yellow solid (17.4 mg, 17.2%). LCMS (ESI) m/z 444.2, [M+H]⁺. ¹H NMR (400 MHz, DMSO-d₆) δ 10.94 (s, 1H), 9.39 (s, 1H), 8.86 (s, 1H), 8.28 (s, 1H), 8.14-8.09 (m, 1H), 8.07-8.01 (m, 1H), 7.63 (d, J=9.6 Hz, 1H), 7.31-7.26 (m, 1H), 6.69 (s, 1H), 3.82-3.75 (m, 4H), 3.16-3.07 (m, 4H), 3.03 (d, J=4.4 Hz, 3H), 2.09-2.00 (m, 1H), 0.87-0.75 (m, 4H).

### Example 797: Synthesis of (S) N-(8-(methylamino)-5-(6-(2-methylmorpholino)pyrazolo[1,5-a]pyridin-2-yl)-2,7-naphthyridin-3-yl)cyclopropanecarboxamide

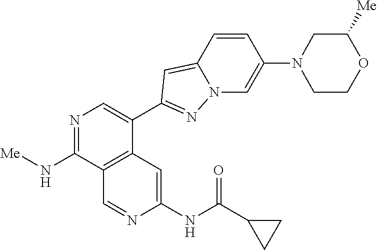 [SMILES: CNc1ncc(-c2cc3ccc(N4CCO[C@@H](C)C4)cn3n2)c2cc(NC(=O)C3CC3)ncc12]

### Step 1: (S)-4-(2-(methoxymethoxy)pyrazolo[1,5-a]pyridin-6-yl)-2-methylmorpholine

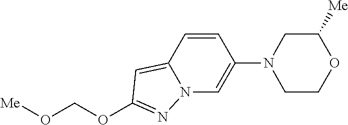 [SMILES: COCOc1cc2ccc(N3CCO[C@@H](C)C3)cn2n1]

To a solution of 6-bromo-2-(methoxymethoxy)pyrazolo[1,5-a]pyridine (Example 696, step 2) (300.1 mg; 1.167 mmol; 1.03 eq.) and (S)-2-methylmorpholine (118.0 mg; 1.167 mmol; 1.01 eq.) in 1,4-dioxane (3 mL) were added Pd-PEPPSI-IHeptCl 3-chloropyridine (113.6 mg; 0.117 mmol; 0.10 eq.) and Cs₂CO₃ (764.2 mg; 2.345 mmol; 2.01 eq.). The resulting mixture was stirred at 100° C. for 14 hours under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide (S)-4-(2-(methoxymethoxy)pyrazolo[1,5-a]pyridin-6-yl)-2-methylmorpholine as a black oil (270.1 mg, 83.4%). LCMS (ESI) m/z 278.1, [M+H]⁺.

### Step 2: (S)-6-(2-methylmorpholino)pyrazolo[1,5-a]pyridin-2-ol

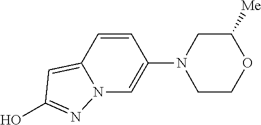 [SMILES: C[C@H]1CN(c2ccc3cc(O)nn3c2)CCO1]

To a solution of (S)-4-(2-(methoxymethoxy)pyrazolo[1,5-a]pyridin-6-yl)-2-methylmorpholine (270.1 mg; 0.974 mmol; 1.00 eq.) in MeOH (3 mL) was added HCl (gas) (4 M in 1,4-dioxane, 3 mL) at 0° C. The resulting mixture was stirred at room temperature for 1 hour. The desired product was detected via LCMS. The resulting mixture was concentrated under reduced pressure. The residue was purified by flash chromatography on silica gel column using 5-8% of MeOH in CH₂Cl₂ (0.05% triethylamine) as eluent to provide (5)-6-(2-methylmorpholino)pyrazolo[1,5-a]pyridin-2-ol as a brown oil (200.2 mg, 88.0%). LCMS (ESI) m/z 234.1, [M+H]⁺.

### Step 3: (S)-6-(2-methylmorpholino)pyrazolo[1,5-a]pyridin-2-yl trifluoromethanesulfonate

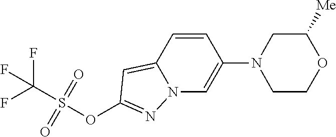 [SMILES: C[C@H]1CN(c2ccc3cc(OS(=O)(=O)C(F)(F)F)nn3c2)CCO1]

To a stirred solution of (S)-6-(2-methylmorpholino)pyrazolo[1,5-a]pyridin-2-ol (200.2 mg; 0.857 mmol; 1.00 eq.) and DIEA (330.5 mg; 2.557 mmol; 2.98 eq.) in CH₂Cl₂ (5 mL) was added Tf₂O (360.2 mg; 1.277 mmol; 1.49 eq.) dropwise at 0° C. The resulting mixture was stirred at room temperature for 1 hour. The resulting mixture was diluted with CH₂Cl₂ (20 mL) and washed with water (2×10 mL). The organic layer was concentrated under reduced pressure to afford (S)-6-(2-methylmorpholino)pyrazolo[1,5-a]pyridin-2-yl trifluoromethanesulfonate as a black oil (300.1 mg, crude). LCMS (ESI) m/z 366.1, [M+H]⁺.

### Step 4: (S)—N-(8-(methylamino)-5-(6-(2-methylmorpholino)pyrazolo[1,5-a]pyridin-2-yl)-2,7-naphthyridin-3-yl)cydopropanecarboxamide

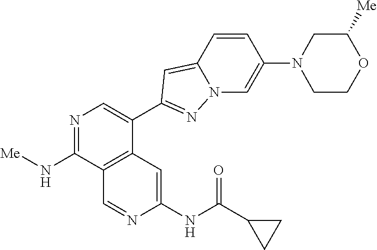 [SMILES: CNc1ncc(-c2cc3ccc(N4CCO[C@@H](C)C4)cn3n2)c2cc(NC(=O)C3CC3)ncc12]

A suspension of (S)-6-(2-methylmorpholino)pyrazolo[1,5-a]pyridin-2-yl trifluoromethanesulfonate (300.0 mg; 0.575 mmol; 1.00 eq.), N-(8-(methylamino)-5-(4,4,5,5-tetramethyl-1,3,2-dioxaborolan-2-yl)-2,7-naphthyridin-3-yl)cyclopropanecarboxamide (212.0 mg; 0.576 mmol; 1.00 eq.), XPhos Pd G₃ (48.6 mg; 0.057 mmol; 0.10 eq.), XPhos (55.1 mg; 0.116 mmol; 0.20 eq.) and K₃PO₄ (366.2 mg; 1.725 mmol; 3.00 eq.) in a mixture of 1,4-dioxane/water (5/1, 3.6 mL) was stirred at 90° C. for 1 hour. The desired product was detected via LCMS. The mixture was allowed to cool down to room temperature. The resulting mixture was concentrated under reduced pressure. The residue was purified by flash chromatography on silica gel column using 2-10% of MeOH in CH₂Cl₂ to afford (S)—N-(8-(methylamino)-5-(6-(2-methylmorpholino)pyrazolo[1,5-a]pyridin-2-yl)-2,7-naphthyridin-3-yl)cyclopropanecarboxamide (87.9 mg, 33.3%) as a yellow solid. LCMS (ESI) m/z 458.2, [M+H]⁺. ¹H NMR (300 MHz, DMSO-d₆) δ 10.92 (s, 1H), 9.39 (s, 1H), 8.86 (s, 1H), 8.28 (s, 1H), 8.21-7.92 (m, 2H), 7.63 (d, J=13.2 Hz, 1H), 7.35-7.19 (m, 1H), 6.69 (s, 1H), 4.01-3.89 (m, 1H), 3.79-3.63 (m, 2H), 3.60-3.49 (m, 1H), 3.49-3.38 (m, 1H), 3.03 (d, J=4.4 Hz, 3H), 2.76-2.61 (m, 1H), 2.42-2.31 (m, 1H), 2.12-1.97 (m, 1H), 1.18 (d, J=7.2 Hz, 3H), 0.89-0.72 (m, 4H).

### Example 798: Synthesis of (1S,2R)-2-methyl-N-(8-(methylamino)-5-(6-((S)-2-methylmorpholino)pyrazolo[1,5-a]pyridin-2-yl)-2,7-naphthyridin-3-yl)cyclopropane-1-carboxamide

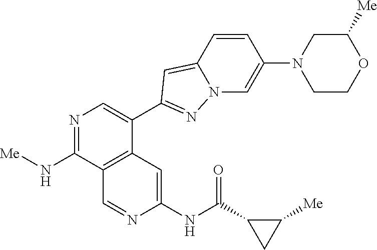 [SMILES: CNc1ncc(-c2cc3ccc(N4CCO[C@@H](C)C4)cn3n2)c2cc(NC(=O)[C@H]3C[C@H]3C)ncc12]

### Step 1: (S)—N¹-methyl-4-(6-(2-methylmorpholino)pyrazolo[1,5-a]pyridin-2-yl)-2,7-naphthyridine-1,6-diamine

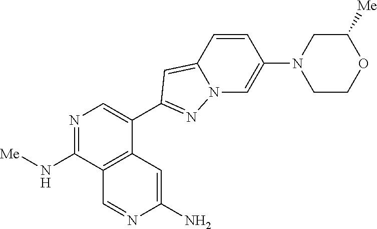 [SMILES: CNc1ncc(-c2cc3ccc(N4CCO[C@@H](C)C4)cn3n2)c2cc(N)ncc12]

To a stirred solution of (S)—N-(8-(methylamino)-5-(6-(2-methylmorpholino)pyrazolo[1,5-a]pyridin-2-yl)-2,7-naphthyridin-3-yl)cyclopropanecarboxamide (Example 797) (60.2 mg; 0.131 mmol; 1.00 eq.) in a mixture solvent of DMSO/MeOH (3/2, 5 mL) was added a solution of NaOH: (52.5 mg; 1.313 mmol; 10.10 eq.) in water (0.5 mL). The resulting mixture was stirred at 60° C. overnight. The desired product was detected via LCMS. The mixture was allowed to cool down to room temperature. The resulting mixture was concentrated under reduced pressure to remove MeOH. The resulting mixture was diluted with water (1 mL), whereupon the precipitated product was collected by filtration and washed with water (0.5 mL). The solids were dried under vacuum to afford (S)—N¹-methyl-4-(6-(2-methylmorpholino)pyrazolo[1,5-a]pyridin-2-yl)-2,7-naphthyridine-1,6-diamine as a yellow solid (37.0 mg, 72.4%). LCMS (ESI) m/z 390.2, [M+H]⁺.

### Step 2: (1S,2R)-2-methyl-N-(8-(methylamino)-5-(6-((S)-2-methylmorpholino)pyrazolo[1,5-a]pyridin-2-yl)-2,7-naphthyridin-3-yl)cyclopropane-1-carboxamide

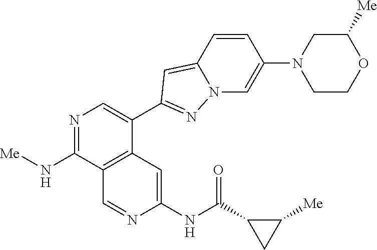 [SMILES: CNc1ncc(-c2cc3ccc(N4CCO[C@@H](C)C4)cn3n2)c2cc(NC(=O)[C@H]3C[C@H]3C)ncc12]

To a stirred solution of (S)—N¹-methyl-4-(6-(2-methylmorpholino)pyrazolo[1,5-a]pyridin-2-yl)-2,7-naphthyridine-1,6-diamine (30.0 mg; 0.077 mmol; 1.00 eq) and (1S,2R)-2-methylcyclopropane-1-carboxylic acid (6.6 mg; 0.066 mmol; 0.86 eq.) in pyridine (1 mL) was added POCl₃ (34.4 mg; 0.224 mmol; 2.91 eq.) dropwise at 0° C. under nitrogen atmosphere. The resulting mixture was stirred at room temperature for 0.5 hour. The desired product was detected via LCMS. The reaction was quenched with water (2 mL) at 0° C. The resulting mixture was concentrated under reduced pressure. The residue was purified by flash chromatography on silica gel column using 2-10% of MeOH in CH₂Cl₂ to afford the crude product. The crude product was purified by flash chromatography on pre-packed C18 column using 20-60% of MeCN in water (10 mmol/L NH₄HCO₃) to provide (1S,2R)-2-methyl-N-(8-(methylamino)-5-(6-((S)-2-methylmorpholino)pyrazolo[1,5-a]pyridin-2-yl)-2,7-naphthyridin-3-yl)cyclopropane-1-carboxamide as a yellow solid (13.0 mg, 35.8%). LCMS (ESI) m/z 472.2, [M+H]⁺. ¹H NMR (400 MHz, DMSO-d₆) δ 10.82 (s, 1H), 9.38 (s, 1H), 8.88 (s, 1H), 8.28 (s, 1H), 8.11 (s, 1H), 8.08-7.99 (m, 1H), 7.64 (d, J=9.6 Hz, 1H), 7.30-7.21 (m, 1H), 6.70 (s, J=0.8 Hz, 1H), 3.99-3.88 (m, 1H), 3.79-3.64 (m, 2H), 3.61-3.50 (m, 1H), 3.49-3.40 (m, 1H), 3.03 (d, J=4.4 Hz, 3H), 2.81-2.74 (m, 1H), 2.40-2.30 (m, 1H), 2.11-2.00 (m, 1H), 1.31-1.22 (m, 1H), 1.17 (d, J=6.4 Hz, 3H), 1.10 (d, J=6.4 Hz, 3H), 1.01-0.92 (m, 1H), 0.87-0.76 (m, 1H).

### Example 799: Synthesis of (1S,2R)—N-(5-((E)-4-(2-cyanoethoxy)styryl)-8-((methyl-d₃)amino)-2,7-naphthyridin-3-yl)-2-methylcyclopropane-1-carboxamide

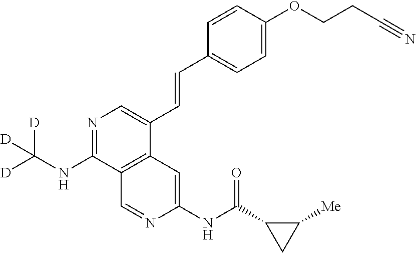 [SMILES: [2H]C([2H])([2H])Nc1ncc(/C=C/c2ccc(OCCC#N)cc2)c2cc(NC(=O)[C@H]3C[C@H]3C)ncc12]

A solution of (1S,2R)—N-(5-bromo-8-((methyl-d₃)amino)-2,7-naphthyridin-3-yl)-2-methylcyclopropane-1-carboxamide (Example 455, Step 1) (60.0 mg; 0.177 mmol; 1.00 eq.), (E)-1,2-bis(tributylstannyl)ethene (112.9 mg; 0.186 mmol; 1.05 eq.) and Pd(PPh₃)₄ (4.1 mg; 0.004 mmol; 0.02 eq.) in toluene (3 mL) was stirred at 11.0° C. for 2 hours under nitrogen atmosphere. The desired product was detected via LCMS. The mixture was allowed to cool down to room temperature. To the above solution was added 3-(4-bromophenoxy)propanenitrile (44.3 mg; 0.196 mmol; 1.10 eq.). The resulting mixture was stirred at 110° C. for another 2 hours under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to afford the crude product. The crude product was recrystallized by CH₂Cl₂/MeOH (5:1, 5 mL) to provide (1S,2R)—N-(5-((E)-4-2-cyanoethoxy)styryl)-8-((methyl-d₃)amino)-2,7-naphthyridin-3-yl)-2-methylcyclopropane-1-carboxamide as a yellow solid (26.0 mg, 33.9%). LCMS (ESI) m/z 431.2, [M+H]⁺. ¹H NMR (400 MHz, DMSO-d₆) δ 10.90 (s, 1H), 9.34 (s, 1H), 8.55 (s, 1H), 8.31 (s, 1H), 7.96 (s, 0.1H), 7.54 (d, 8.4 Hz, 2H), 7.25 (d, J=16.0 Hz, 1H), 7.10-6.82 (m, 3H), 4.23 (t, J=6.0 Hz, 2H), 3.03 (t, J=6.0 Hz, 2H), 2.12-2.03 (m, 0.14), 1.32-1.28 (m, 1H), 1.14 (d, J 6.0 Hz, 3H), 1.04-0.96 (m, 1H), 0.89-0.82 (m, 1H).

### Example 800: Synthesis of N-(6-(hydroxymethyl)-54(5-methoxypyridin-2-yl)ethynyl)-8-(methylamino)-2,7-naphthyridin-3-yl)cyclopropanecarboxamide

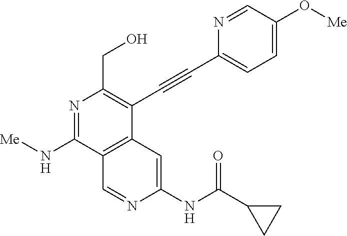 [SMILES: CNc1nc(CO)c(C#Cc2ccc(OC)cn2)c2cc(NC(=O)C3CC3)ncc12]

### Step 1: N-(8-(methylamino)-6-vinyl-2,7-naphthyridin-3-yl)cyclopropanecarboxamide

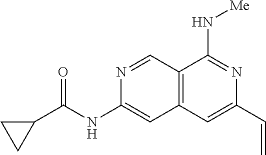 [SMILES: C=Cc1cc2cc(NC(=O)C3CC3)ncc2c(NC)n1]

A solution of 6-(cyclopropanecarboxamido)-1-(methylamino)-2,7-naphthyridin-3-yl trifluoromethanesulfonate(E(Example 780, step 6)

(100.0 mg; 0.256 mmol; 1.00 eq.) and 4,4,5,5-tetramethyl-2-vinyl-1,3,2-dioxaborolane (47.5 mg; 0.308 mmol; 1.20 eq.), Pd(DtBPF)Cl₂ (33.5 mg, 0.051 mmol; 0.20 eq.), K₃PO₄ (109.1 mg; 0.514 mmol; 2.01 eq.) in a mixture solvent of 1,4-dioxane/water (5:1, (5 mL) was stirred at 90° C. for 2 hours under nitrogen atmosphere. The desired product was detected via LCMS. The resulting mixture was concentrated under reduced pressure. The residue was purified by flash chromatography on silica gel column using 50-70% of EtOAc in petroleum ether as eluent to provide N-(8-(methylamino)-6-vinyl-2,7-naphthyridin-3-yl)cyclopropanecarboxamide as an off-white solid (65.0 mg, 94.5%). LCMS (ESI) m/z 269.1, [M+H]⁺.

### Step 2: N-(6-formyl-8-(methylamino)-2,7-naphthyridin-3-yl)cyclopropanecarboxamide

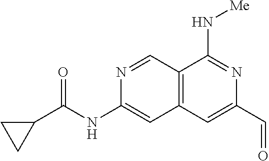 [SMILES: CNc1nc(C=O)cc2cc(NC(=O)C3CC3)ncc12]

A solution of N-(8-(methylamino)-6-vinyl-2,7-naphthyridin-3-yl)cyclopropanecarboxamide (65.0 mg; 0.242 mmol; 1.00 eq.), 2,6-dimethylpyridine (51.9 mg; 0.484 mmol; 2.00 eq.), K₂OsO₄·2H₂O (8.9 mg; 0.024 mmol; 0.10 eq.) and NaIO₄ (207.2 mg; 0.969 mmol; 4.00 eq.) in a mixture solvent of 1,4-dioxane/water (1:1, 6 mL) was stirred at 0° C. overnight under nitrogen atmosphere. The precipitated solids were collected by filtration and washed with water (3×10 mL). The residue was purified by flash chromatography on pre-packed C18 column using 40-60% of MeOH in water. (10 mmol/L NH₄HCO₃) as eluent to provide N-(6-formyl-8-(methylamino)-2,7-naphthyridin-3-yl)cyclopropanecarboxamide as a yellow solid (30.0 mg, 45.8%). LCMS (ESI) m/z 271.1, [M+H]⁺.

### Step 3: N-(6-(hydroxymethyl)-8-(methylamino)-2,7-naphthyridin-3-yl)cycloprapanecarboxamide

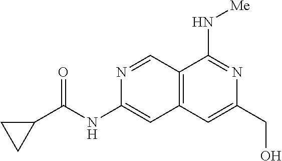 [SMILES: CNc1nc(CO)cc2cc(NC(=O)C3CC3)ncc12]

To a solution of N-(6-formyl-8-(methylamino)-2,7-naphthyridin-3-yl)cyclopropanecarboxamide (25.0 mg; 0.092 mmol; 1.00 eq.) in a mixture solvent of MeOH/THF (1:1, 3 mL) was added NaBH₄ (5.2 mg; 0.137 mmol; 1.49 eq.) at 0° C. under nitrogen atmosphere. The resulting mixture was stirred at room temperature for 30 minutes. The desired product was detected via LCMS.

The reaction was quenched by the addition of ice water (3 mL) at 0° C. The resulting mixture was concentrated under reduced pressure. The residue was purified by flash chromatography on pre-packed C18 column using 20-50% of MeOH in water (10 mmol/L NH₄HCO₃) as eluent to provide N-(6-(hydroxymethyl)-8-(methylamino)-2,7-naphthyridin-3-yl)cyclopropanecarboxamide as an off-white solid (21.0 mg, 83.3%). LCMS (ESI) m/z 273.1, [M+H]⁺.

### Step 4: N-(6-(hydroxymethyl)-5-iodo-8-(methylamino)-2,7-naphthyridin-3-yl)cyclopropane carboxamide

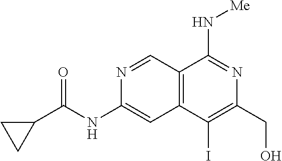 [SMILES: CNc1nc(CO)c(I)c2cc(NC(=O)C3CC3)ncc12]

To a stirred solution of N-(6-(hydroxymethyl)-8-(methylamino)-2,7-naphthyridin-3-yl)cyclopropanecarboxamide (21.0 mg; 0.077 mmol; 1.00 eq.) in DMF (2 mL) was added NIS (13.4 mg; 0.060 mmol; 0.77 eq.) at 0° C. under nitrogen atmosphere. The resulting mixture was stirred at 0° C. for 1 hour. The desired product was detected via LCMS. The mixture was purified by flash chromatography on pre-packed C18 column using 40-60% of MeOH in water (10 mmol/L NH₄HCO₃) as eluent to provide N-(6-(hydroxymethyl)-5-iodo-8-(methylamino)-2,7-naphthyridin-3-yl)cyclopropanecarboxamide as a yellow solid (29.0 mg, 94.4%). LCMS (ESI) m/z 399.0, [M+H]⁺.

### Step 5: N-(6-(hydroxymethyl)-5-((5-methoxypyridin-2-yl)ethynyl)-8-(methylamino)-2,7-naphthyridin-3-yl)cyclopropanecarboxamide

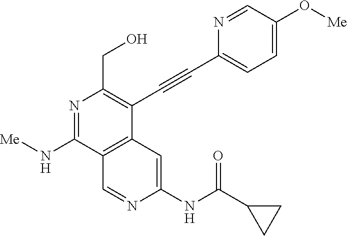 [SMILES: CNc1nc(CO)c(C#Cc2ccc(OC)cn2)c2cc(NC(=O)C3CC3)ncc12]

A solution of N-(6-(hydroxymethyl)-5-iodo-8-(methylamino)-2,7-naphthyridin-3-yl)cyclopropanecarboxamide (25.0 mg; 0.063 mmol; 1.00 eq.), 2-ethynyl-5-methoxypyridine (41.8 mg; 0.314 mmol; 5.00 eq.), Pd(PPh₃)₄ (14.5 mg; 0.013 mmol; 0.20 eq.), triethylamine (44.5 mg; 0.440 mmol; 7.00 eq.) and CuI (2.4 mg; 0.013 mmol; 0.20 eq.) in DMA (2 mL) was stirred at 80° C. overnight under nitrogen atmosphere. The desired product was detected via LCMS. The resulting mixture was diluted with water (10 mL) and extracted with EtOAc (3×10 mL). The combined organic layers were washed with brine (3×10 mL), dried over anhydrous Na₂SO₄. After filtration, the filtrate was concentrated under reduced pressure. The residue was purified by flash chromatography on silica gel column using 5-10% of MeOH in CH₂Cl₂ as eluent to provide the crude product. This crude product was purified by Prep-Achiral-SFC (Column: GreenSep Naphthyl, 3×25 cm, 5 μm; Mobile Phase A: CO₂, Mobile Phase B: MeOH (0.1% 2 M NH₃-MeOH); Flow rate: 75 mL/min; Gradient: isocratic 36% B; Column Temperature(° C.): 35; Back Pressure(bar): 100; Wave Length: 254 nm; RT(min): 5.67) to provide N-(6-(hydroxymethyl)-5-((5-methoxypyridin-2-yl)ethynyl)-8-(methylamino)-2,7-naphthyridin-3-yl)cyclopropanecarboxamide as a yellow solid (5.0 mg, 19.7%). LCMS (ESI) m/z 404.2, [M+H]⁺. ¹H NMR (400 MHz, DMSO-d₆) δ 11.05 (s, 1H), 9.36 (s, 1H), 8.65 (s, 1H), 8.49-8.36 (m, 1H), 8.33 (d, J=2.8 Hz, 1H), 7.62 (d, J=8.8 Hz, 1H), 7.48 (dd, J=8.4, 2.8 Hz, 1H), 4.92 (t, J=5.6 Hz, 1H), 4.71 (d, J=5.6 Hz, 2H), 3.89 (s, 3H), 3.09 (d, J=4.4 Hz, 3H), 2.15-2.02 (m, 1H), 0.89-0.78 (m, 4H).

### Examples 801-806

Each compound in Table 27 below was prepared using a similar experimental procedure to prepare Example 595, using N-(5-ethynyl-8-(methylamino)-2,7-naphthyridin-3-yl)cyclopropanecarboxamide as the common intermediate and appropriate aryl halide.

TABLE 27

| Example | Structure and Name | MS and ¹H NMR |
|---|---|---|
| Example 801 | 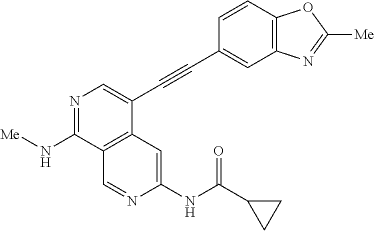 [SMILES: CNc1ncc(C#Cc2ccc3oc(C)nc3c2)c2cc(NC(=O)C3CC3)ncc12] N-(8-(methylamino)-5-((2- methylbenzo[d]oxazol-5-yl)ethynyl)- 2,7-naphthyridin-3- yl)cyclopropanecarboxamide | LCMS (ESI) m/z 398.1, [M + H]⁺. ¹H NMR (400 MHz, DMSO-d₆) δ 11.10 (s, 1H), 9.38 (s, 1H), 8.70 (s, 1H), 8.39-8.31 (m, 1H), 8.30 (s, 1H), 7.84 (d, J = 1.4 Hz, 1H), 7.75 (d, J = 8.4 Hz, 1H), 7.54 (dd, J = 8.4, 1.6 Hz, 1H), 3.03 (d, J = 4.4 Hz, 3H), 2.65 (s, 3H), 2.14-2.04 (m, 1H), 1.10-0.86 (m, 4H). |
| Example 802 | 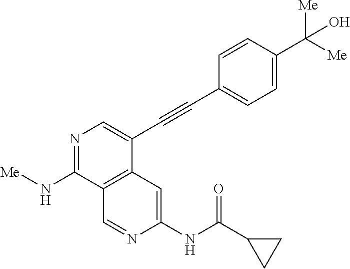 [SMILES: CNc1ncc(C#Cc2ccc(C(C)(C)O)cc2)c2cc(NC(=O)C3CC3)ncc12] N-(5-((4-(2-hydroxypropan-2- yl)phenyl)ethynyl)-8-(methylamino)- 2,7-naphthyridin-3- yl)cyclopropanecarboxamide | LCMS (ESI) m/z 401.2, [M + H]⁺. ¹H NMR (400 MHz, DMSO-d₆) δ 11.08 (s, 1H), 9.37 (s, 1H), 8.67 (s, 1H), 8.43-8.29 (m, 1H), 8.27 (s, 1H), 7.76-7.15 (m, 4H), 5.11 (s, 1H), 3.02 (d, J = 4.4 Hz, 3H), 2.15- 2.01 (m, 1H), 1.45 (s, 6H), 0.99- 0.75 (m, 4H). |
| Example 803 | 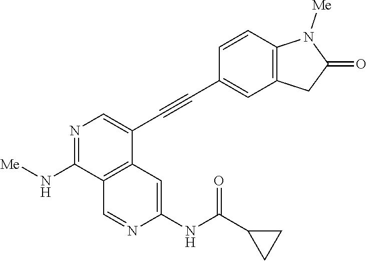 [SMILES: CNc1ncc(C#Cc2ccc3c(c2)CC(=O)N3C)c2cc(NC(=O)C3CC3)ncc12] N-(5-((1-methyl-2-oxoindolin-5- yl)ethynyl)-8-(methylamino)-2,7- naphthyridin-3- yl)cyclopropanecarboxamide | LCMS (ESI) m/z 412.1, [M + H]⁺. ¹H NMR (400 MHz, DMSO-d₆) δ 11.07 (s, 1H), 9.37 (s, 1H), 8.65 (s, 1H), 8.46-8.28 (m, 1H), 8.25 (s, 1H), 7.50 (d, J = 8.0 Hz, 1H), 7.45 (s, 1H), 7.06 (d, J = 8.0 Hz, 1H), 3.60 (s, 2H), 3.15 (s, 3H), 3.02 (d, J = 4.4 Hz, 3H), 2.14-1.98 (m, 1H), 0.97-0.71 (m, 4H). |
| Example 804 | 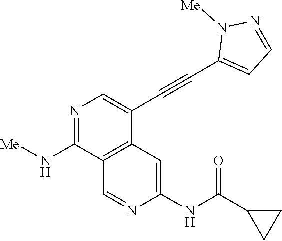 [SMILES: CNc1ncc(C#Cc2ccnn2C)c2cc(NC(=O)C3CC3)ncc12] N-(5-((1-methyl-1H-pyrazol-5- yl)ethynyl)-8-(methylamino)-2,7- naphthyridin-3- yl)cyclopropanecarboxamide | LCMS (ESI) m/z 347.2, [M + H]⁺. ¹H NMR (400 MHz, DMSO-d₆) δ 11.11 (s, 1H), 9.39 (s, 1H), 8.61 (s, 1H), 8.52-8.40 (m, 1H), 8.33 (s, 1H), 7.52 (d, J = 2.0 Hz, 1H), 6.59 (d, J = 2.0 Hz, 1H), 4.00 (s, 3H), 3.03 (d, J = 4.4 Hz, 3H), 2.12- 2.02 (m, 1H), 0.92-0.78 (m, 4H). |
| Example 805 | 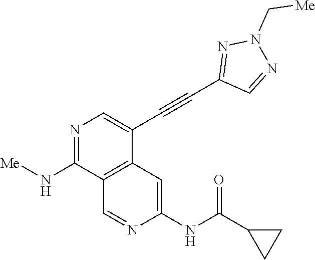 [SMILES: CCn1ncc(C#Cc2cnc(NC)c3cnc(NC(=O)C4CC4)cc23)n1] N-(5-((2-ethyl-2H-1,2,3-triazol-4- yl)ethynyl)-8-(methylamino)-2,7- naphthyridin-3- yl)cyclopropanecarboxamide | LCMS (ESI) m/z 362.2, [M + H]⁺. ¹H NMR (400 MHz, DMSO-d₆) δ 11.11 (s, 1H), 9.39 (s, 1H), 8.62- 8.39 (m, 2H), 8.31 (s, 1H), 8.05 (s, 1H), 4.57-4.39 (m, 2H), 3.04 (d, J = 4.4 Hz, 3H), 2.14-1.98 (m, 1H), 1.49 (t, J = 7.2 Hz, 3H), 0.91- 0.77 (m, 4H). |
| Example 806 | 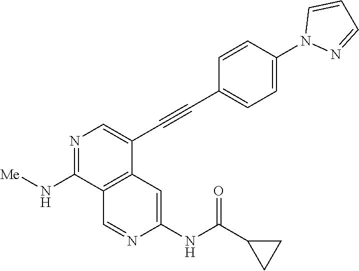 [SMILES: CNc1ncc(C#Cc2ccc(-n3cccn3)cc2)c2cc(NC(=O)C3CC3)ncc12] N-(5-((4-(1H-pyrazol-1- yl)phenyl)ethynyl)-8-(methylamino)- 2,7-naphthyridin-3- yl)cyclopropanecarboxamide | LCMS (ESI) m/z 409.2, [M + H]⁺. ¹H NMR (400 MHz, DMSO-d₆) δ 11.09 (s, 1H), 9.38 (s, 1H), 8.69 (s, 1H), 8.59 (d, J = 2.4 Hz, 1H), 8.48- 8.32 (m, 1H), 8.30 (s, 1H), 7.95 (d, J = 8.4 Hz, 2H), 7.80 (s, 1H), 7.67 (d, J = 8.4 Hz, 2H), 6.59 (s, 1H), 3.03 (d, J = 4.4 Hz, 3H), 2.22- 1.95 (m, 1H), 1.07-0.70 (m, 4H). |

### Example 807: Synthesis of N-(8-(methylamino)-5-((1,2,3,4-tetrahydrobenzo[4,5]imidazo[1,2-a]pyridin-8-yl)ethynyl)-2,7-naphthyridin-3-yl)cyclopropanecarboxamide

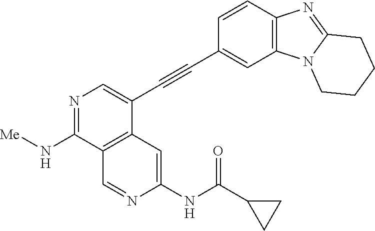 [SMILES: CNc1ncc(C#Cc2ccc3nc4n(c3c2)CCCC4)c2cc(NC(=O)C3CC3)ncc12]

### Step 1: 1-(5-bromo-2-nitrophenyl)piperidine

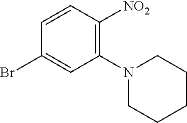 [SMILES: O=[N+]([O-])c1ccc(Br)cc1N1CCCCC1]

A solution of 4-bromo-2-fluoro-1-nitrobenzene (1.00 g; 4.546 mmol; 1.50 eq.), Cs₂CO₃ (1.97 g; 6.046 mmol; 1.33 eq.) and piperidine (0.26 g; 3.031 mmol; 1.00 eq.) in DMF (4 mL) was stirred at 90° C. for 2 hours under nitrogen atmosphere. The resulting mixture was diluted with EtOAc (30 mL) and washed with brine (2×10 mL). The organic layer was dried over anhydrous Na₂SO₄. After filtration, the filtrate was concentrated under reduced pressure. The residue was purified by flash chromatography on silica gel column using 0-35% of EtOAc in petroleum ether as eluent to provide 1-(5-bromo-2-nitrophenyl)piperidine as an off-white solid (900.0 mg, 69.5%). LCMS (ESI) m/z 285.1, [M+H]⁺.

### Step 2: 4-bromo-2-(piperidin-1-yl) aniline

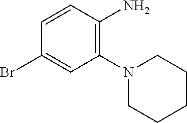 [SMILES: Nc1ccc(Br)cc1N1CCCCC1]

A solution of 1-(5-bromo-2-nitrophenyl)piperidine (700.0 mg; 2.455 mmol; 1.00 eq.), NH₄Cl (1.05 g; 19.640 mmol; 8.00 eq.) and Fe powder (195.8 mg; 3.505 mmol; 5.00 eq.) in a mixture solvent of EtOH/water (5:1, 6 mL) was stirred at 80° C. for 2 hours under nitrogen atmosphere. The insoluble solids were filtered out, the filtrate was concentrated under reduced pressure to afford 4-bromo-2-(piperidin-1-yl) aniline as a brown solid (683.0 mg, crude). LCMS (ESI) m/z 255.0, [M+H]⁺.

### Step 3: 8-bromo-1,2,3,4-tetrahydrobenzo[4,5]imidazo[1,2-a]pyridine

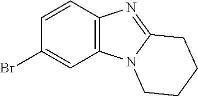 [SMILES: Brc1ccc2nc3n(c2c1)CCCC3]

To a stirred solution of 4-bromo-2-(piperidin-1-yl) aniline (350.0 mg; 1.372 mmol; 1.00 eq.) in CH₂Cl₂ (10 mL) were added TFA (1.2 mL) and H₂O₂(30%) (0.2 mL) dropwise at room temperature under nitrogen atmosphere. The resulting mixture was stirred at 40° C. for 30 minutes. The desired product was detected via LCMS. The solution was cooled to room temperature, quenched with saturated Na₂S₂O₃ solution. The resulting mixture was washed with saturated sodium carbonate solution (1×5 mL) and water (3×5 mL). The organic layer was dried over anhydrous Na₂SO₄. After filtration, the filtrate was concentrated under reduced pressure. The residue was purified by flash chromatography on silica gel column using 10-40% of EtOAc in petroleum ether as eluent to provide 8-bromo-1,2,3,4-tetrahydrobenzo[4,5]imidazo[1,2-a]pyridine as an off-white solid (250.0 mg, 72.5%). LCMS (ESI) m/z 251.1, [M+H]⁺.

### Step 4: N-(8-(methylamino)-5-((1,2,3,4-tetrahydrobenzo[4,5]imidazo[1,2-a]pyridin-8-yl)ethynyl)-2,7-naphthyridin-3-yl)cyclopropanecarboxamide

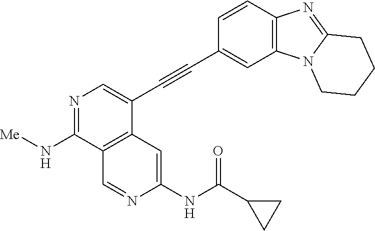 [SMILES: CNc1ncc(C#Cc2ccc3nc4n(c3c2)CCCC4)c2cc(NC(=O)C3CC3)ncc12]

N-(8-(methylamino)-5-((1,2,3,4-tetrahydrobenzo[4,5]imidazo[1,2-a]pyridin-8-yl)ethynyl)-2,7-naphthyridin-3-yl)cyclopropanecarboxamide was synthesized using a similar procedure that was previously described in Example 595, by using N-(5-ethynyl-8-(methylamino)-2,7-naphthyridin-3-yl)cyclopropanecarboxamide and 8-bromo-1,2,3,4-tetrahydrobenzo[4,5]imidazo[1,2-a]pyridine as the starting material. LCMS (ESI) m/z 437.2, [M+H]⁺. ¹H NMR (400 MHz, DMSO-d₆) δ 11.12 (s, 1H), 9.38 (s, 1H), 8.78 (s, 1H), 8.35-8.22 (m, 2H), 7.84 (s, 1H), 7.65-7.53 (m, 1H), 7.35 (d, J=° 6.0 Hz, 1H), 4.20-4.12 (m, 2H), 3.08-2.94 (m, 5H), 2.12-2.03 (m, 3H), 2.00-1.91 (m, 2H), 0.96-0.82 (m, 4H).

### Example 808: Synthesis of N-(5-((1,2-dihydrobenzo[4,5]imidazo[1,2-a]pyridin-8-yl)ethynyl)-8-(methylamino)-2,7-naphthyridin-3-yl)cyclopropanecarboxamide

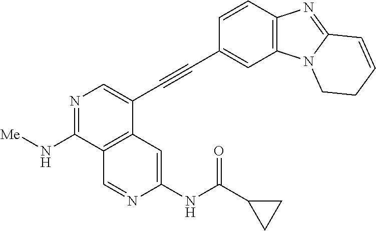 [SMILES: CNc1ncc(C#Cc2ccc3nc4n(c3c2)CCC=C4)c2cc(NC(=O)C3CC3)ncc12]

### Step 1: 4-(fluoro-1-(5-iodo-2-nitrophenyl)piperidine

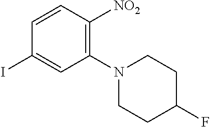 [SMILES: O=[N+]([O-])c1ccc(I)cc1N1CCC(F)CC1]

To a solution of 2-fluoro-4-iodo-1-nitrobenzene (500.0 mg; 1.873 mmol; 1.00 eq.) in DMF (4 mL) was added 4-fluoropiperidine hydrochloride (174.0 mg; 1.246 mmol; 0.67 eq.) and Cs₂CO₃ (816.2 mg; 2.505 mmol; 1.34 eq.) at room temperature. The reaction was stirred at 90° C. for 2 hours under nitrogen atmosphere. The desired product was detected via LCMS. The reaction was diluted with water. (20 mL) and extracted with EtOAc (2×50 mL). The combined organic layers were washed with brine (3×10 mL), dried over anhydrous Na₂SO₄. After filtration, the filtrate was concentrated under reduced pressure. The residue was purified by flash chromatography on silica gel column using 10-30% of CH₂Cl₂ in petroleum ether as eluent to provide 4-fluoro-1-(5-iodo-2-nitrophenyl)piperidine as yellow oil (465.0 mg, 70.9%). LCMS (ESI) m/z 351.0, [M+H]⁺.

### Step 2: 2-(4-fluoropiperidin-1-yl)-4-iodoaniline

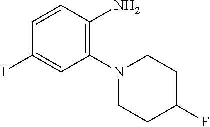 [SMILES: Nc1ccc(I)cc1N1CCC(F)CC1]

To a solution of 4-fluoro-1-(5-iodo-2-nitrophenyl)piperidine (465.0 mg; 1.328 mmol; 1.00 eq.) in a mixture solvent of EtOH/water (5:1, 7.2 mL) was added Fe powder (372.0 mg; 6.661 mmol; 5.02 eq.) and NH₄₀ (568.6 mg; 10.630 mmol; 8.00 eq.) at room temperature. The reaction was stirred at 80° C. for 2 hours under nitrogen atmosphere. The desired product was detected via LCMS. The solids were filtered out and washed with EtOAc. The filtrate was concentrated under vacuum. The residue was purified by flash chromatography on silica gel column using 10-30% of EtOAc in petroleum ether as eluent to provide 2-(4-fluoropiperidin-1-yl)-4-iodoaniline as black oil (336.0 mg, 79.0%). LCMS (ESI) m/z 321.0, [M+H]⁺.

### Step 3: 3-fluoro-8-iodo-1,2,3,4-tetrahydrobenzo[4,5]imidazo[1,2-a]pyridine

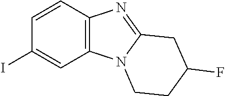 [SMILES: FC1CCn2c(nc3ccc(I)cc32)C1]

To a solution of 2-(4-fluoropiperidin-1-yl)-4-iodoaniline (336.0 mg; 1.050 mmol; 1.00 eq.) in CH₂Cl₂ (8.2 mL) was added TFA (1 mL) and H₂O₂ (30%, 0.2 mL) dropwise at room temperature. The reaction was stirred at 40° C. for 1 hour under nitrogen atmosphere. The desired product was detected via LCMS. The reaction was quenched with saturated Na₂S₂O₃ solution, neutralized to pH==7 with Na₂CO₃ aqueous solution. The resulting mixture was extracted with EtOAc (2×50 mL). The combined organic layers were washed with brine (3×10 mL), dried over anhydrous Na₂SO₄. After filtration, the filtrate was concentrated under reduced pressure. The residue was purified by flash chromatography on silica gel column using 10-30% of EtOAc in petroleum ether as eluent to provide 3-fluoro-8-iodo-1,2,3,4-tetrahydrobenzo[4,5]imidazo[1,2-a]pyridine as white solid (75.0 mg, 22.6%). LCMS (ESI) m/z 317.0, [M+H]⁺.

### Step 4: N-(5-((1,2-dihydrobenzo[4,5]imidazo[1,2-a]pyridin-8-yl)ethynyl)-8-(methylamino)-2,7-naphthyridin-3-yl)cyclopropanecarboxamide

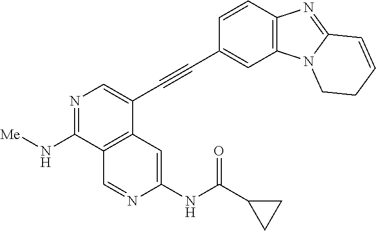 [SMILES: CNc1ncc(C#Cc2ccc3nc4n(c3c2)CCC=C4)c2cc(NC(=O)C3CC3)ncc12]

A solution of 3-fluoro-8-iodo-1,2,3,4-tetrahydrobenzo[4,5]imidazo[1,2-a]pyridine (65.0 mg; 0.206 mmol; 1.00 eq.), N-(5-ethynyl-8-(methylamino)-2,7-naphthyridin-3-yl)cyclopropanecarboxamide (82.0 mg; 0.308 mmol; 1.50 eq.), XPhos Pd G₃ (57.2 mg; 0.068 mmol; 0.33 eq.), XPhos (29.4 mg; 0.062 mmol; 0.30 eq.), Et₃N (83.1 mg; 0.821 mmol; 3.99 eq.) and CuI (11.7 mg; 0.061 mmol; 0.30 eq.) in DMF (3 mL) was stirred at 60° C. for 3 hours under nitrogen atmosphere. The desired product was detected via LCMS. The resulting mixture was concentrated under vacuum. The residue was purified by flash chromatography on silica gel column using 2-10% of MeOH in CH₂Cl₂ as eluent to provide a crude product. The crude product was purified by flash chromatography on pre-packed C18 column using 20-100% of MeOH in water (10 mmol/L NH₄HCO₃) to provide N-(5-((1,2-dihydrobenzo[4,5]imidazo[1,2-a]pyridin-8-yl)ethynyl)-8-(methylamino)-2,7-naphthyridin-3-yl)cyclopropanecarboxamide as a yellow solid (41.2 mg, 46.1%. LCMS (ESI) m/z 435.2, [M+H]⁺. ¹H NMR (400 MHz, DMSO-d₃) δ 11.11 (s, 1H), 9.38 (s, 1H), 8.77 (s, 1H), 8.41-8.11 (m, 2H), 7.86 (d, J=1.6 Hz, 1H), 7.61 (d, J=8.0 Hz, 1H), 7.48-7.18 (m, 1H), 6.66 (d, J=10.4 Hz, 1H), 6.63-6.52 (m, 1H), 4.31 (t, J=7.6 Hz, 2H), 3.03 (d, J=4.4 Hz, 3H), 2.81-2.69 (m, 2H), 2.17-2.01 (m, 1H) 0.98-0.81 (m, 4H).

### Example 809: Synthesis of N-(5-((3-fluoro-1,2-dihydrobenzo[4,5]imidazo[1,2-a]pyridin-8-yl)ethynyl)-8-(methylamino)-2,7-naphthyridin-3-yl)cyclopropanecarboxamide

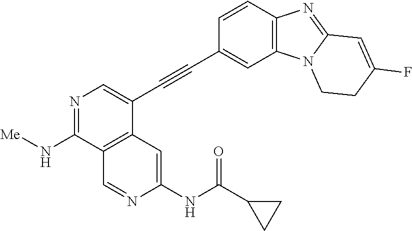 [SMILES: CNc1ncc(C#Cc2ccc3nc4n(c3c2)CCC(F)=C4)c2cc(NC(=O)C3CC3)ncc12]

### Step 1: 1-(5-bromo-2-nitrophenyl)-4,4-difluoropiperidine

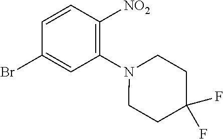 [SMILES: O=[N+]([O-])c1ccc(Br)cc1N1CCC(F)(F)CC1]

To a solution of 4-bromo-2-fluoro-1-nitrobenzene (1.00 g; 4.546 mmol; 1.00 eq.) in DMF (8 mL) was added Cs₂CO₃ (2.00 g; 6.138 mmol; 1.35 eq.) and 4,4-difluoropiperidine hydrochloride (478.0 mg; 3.033 mmol; 0.67 eq.) at room temperature. The reaction was stirred at 90° C. for 2 hours under nitrogen atmosphere. The desired product was detected via LCMS. The reaction was diluted with water (20 mL) and extracted with EtOAc (2×100 mL). The combined organic layers were washed with brine (3×10 mL) and dried over anhydrous Na₂SO₄. After filtration, the filtrate was concentrated under reduced pressure. The residue was purified by flash chromatography on silica gel column using 10-30% of CH₂Cl₂ in petroleum ether as eluent to provide 1-(5-bromo-2-nitrophenyl)-4,4-difluoropiperidine as yellow solid (900.0 mg, 61.6%). LCMS (ESI) m/z 321.0, [M+H]⁺.

### Step 2: 4-bromo-2-(4,4-difluoropiperidin-1-yl) aniline

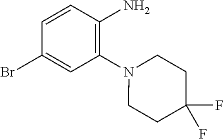 [SMILES: Nc1ccc(Br)cc1N1CCC(F)(F)CC1]

To a solution of 1-(5-bromo-2-nitrophenyl)-4,4-difluoropiperidine (690.0 mg; 2.149 mmol; 1.00 eq.) in a mixture solvent of EtOH/water (5:1, 7.2 mL) was added Fe powder (604.0 mg; 10.816 mmol; 5.03 eq.) and NH₄C1(923.0 mg; 17.256 mmol; 8.03 eq.) at room temperature. The reaction was stirred at 80° C. for 2 hours under nitrogen atmosphere. The desired product was detected via LCMS. The solids were filtered out and washed with. EtOAc. The filtrate was concentrated to afford 4-bromo-2-(4,4-difluoropiperidin-1-yl) aniline as a brown solid (850.0 mg crude). LCMS (ESI) m/z 291.0, [M+H]⁺.

### Step 3: 8-bromo-3,3-difluoro-1,2,3,4-tetrahydrobenzo[4,5]imidazo[1,2-a]pyridine

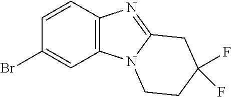 [SMILES: FC1(F)CCn2c(nc3ccc(Br)cc32)C1]

To a solution of 4-bromo-2-(4,4-difluoropiperidin-1-yl) aniline (400.0 mg; 1.374 mmol; 1.00 eq.) in CH₂Cl₂ (9.6 mL) was added TFA (1.2 mL) and H₂O₂ (30%, 0.24 mL) dropwise at room temperature. The reaction was stirred at 40° C. for 2 hours under nitrogen atmosphere. The desired product was detected via LCMS. The reaction was quenched with saturated Na₂S₂O₃ solution at 0° C., basified to pH=7 with Na₂CO₃ aqueous solution. The aqueous phase was extracted with EtOAc (2×50 mL). The combined organic layers were washed with brine (3×1.0 mL), dried over anhydrous Na₂SO₄. After filtration, the filtrate was concentrated under reduced pressure. The residue was purified by flash chromatography on silica gel column using 10-30% of EtOAc in petroleum ether as eluent to provide 8-bromo-3,3-difluoro-1,2,3,4-tetrahydrobenzo[4,5]imidazo[1,2-a]pyridine as a white solid (163.0 mg, 41.3%). LCMS (ESI) m/z 287.0, [M+H]⁺.

### Step 4: N-(5-((3-fluoro-1,2-dihydrobenzo[4,5]imidazo[1,2-a]pyridin-8-yl)ethynyl)-8-(methylamino)-2,7-naphthyridin-3-yl)cyclopropanecarboxamide

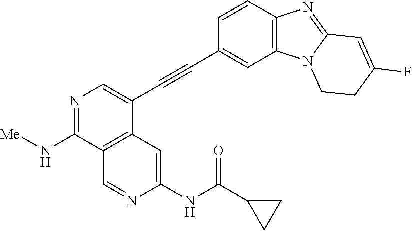 [SMILES: CNc1ncc(C#Cc2ccc3nc4n(c3c2)CCC(F)=C4)c2cc(NC(=O)C3CC3)ncc12]

A solution of 8-bromo-3,3-difluoro-1,2,3,4-tetrahydrobenzo[4,5]imidazo[1,2-a]pyridine (80.0 mg; 0.279 mmol; 1.00 eq.), N-(5-ethynyl-8-(methylamino)-2,7-naphthyridin-3-yl)cyclopropanecarboxamide (57.0 mg; 0.214 mmol; 0.77 eq.), XPhos Pd G₃ (50.6 mg. 0.060 mmol. 0.21 eq.), XPhos (28.5 mg; 0.060 mmol; 0.21 eq.), Et₃N (80.5 mg; 0.796 mmol; 2.85 eq.) and CuI (11.4 mg; 0.060 mmol; 0.21 eq.) in DMF (3 mL) was stirred at 110° C. for 2 hours under nitrogen atmosphere. The desired product was detected via LCMS. The resulting mixture was concentrated under vacuum. The residue was purified by flash chromatography on silica gel column using 2-10% of MeOH in CH₂Cl₂ as eluent to provide the crude product. The crude product was purified by Prep-Achiral-SFC (Column: Torus 2-PIC OBD, 3×15 cm, 5 μm; Mobile Phase A: CO₂, Mobile Phase B: MeOH (0.1% 2 M NH₃-MeOH); Flow rate: 60 mL/min; Gradient: isocratic 33% B; Column Temperature(° C.): 35; Back Pressure(bar): 1.00; Wave Length: 254 nm) to afford N-(5-((3-fluoro-1,2-dihydrobenzo[4,5]imidazo[1,2-a]pyridin-8-yl)ethynyl)-8-(methylamino)-2,7-naphthyridin-3-yl)cyclopropanecarboxamide as a yellow solid (27.9 mg, 22.1%). LCMS (ESI) m/z 453.2, [M+H]⁺. ¹H NMR (400 MHz, DMSO-d₆) δ 11.12 (s, 1H), 9.38 (s, 1H), 8.76 (s, 1H), 8.10-8.29 (m, 1H), 8.28 (s, 1H), 7.83 (d, J=1.6 Hz, 1H), 7.59 (d, J=8.4 Hz, 1H), 7.34 (dd, J=8.4, 1.6 Hz, 1H), 6.46 (d, J=12.0 Hz, 1H), 4.55-4.30 (m, 2H), 3.17-3.05 (m, 2H), 3.03 (d, J=4.4 Hz, 3H), 2.19-2.03 (m, 1H), 1.01-0.70 (m, 4H).

### Example 810: Synthesis of N-(5-((2-(fluoromethyl)-1-methyl-1H-benzo[d]imidazol-6-yl)ethynyl)-8-(methylamino)-2,7-naphthyridin-3-yl)cyclopropanecarboxamide

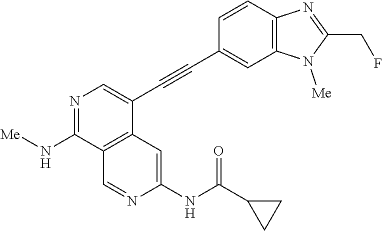 [SMILES: CNc1ncc(C#Cc2ccc3nc(CF)n(C)c3c2)c2cc(NC(=O)C3CC3)ncc12]

### Step 1: (6-bromo-1-methyl-1H-benzo[d]imidazol-2-yl)methanol

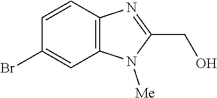 [SMILES: Cn1c(CO)nc2ccc(Br)cc21]

A solution of 5-bromo-N¹-methylbenzene-1,2-diamine (200.0 mg; 0.995 mmol; 1.00 eq.) and 2-hydroxyacetic acid (378.2 mg; 4.973 mmol; 5.00 eq.) in HCl (3 M in water, 30 mL) was stirred at 100° C. for 16 hours. The desired product was detected via LCMS. The mixture was neutralized to pH=7 with 4 M NaOH aqueous solution. The resulting mixture was extracted with EtOAc (3×15 mL). The combined organic layers were washed with brine (3×15 mL), dried over anhydrous Na₂SO₄. After filtration, the filtrate was concentrated under reduced pressure. The residue was purified by flash chromatography on silica gel column using 15-20% of MeOH in CH₂Cl₂ as eluent to provide (6-bromo-1-methyl-1H-benzo[d]imidazol-2-yl)methanol as a brown solid (210.0 mg, 87.5%). LCMS (ESI) m/z 241.1, [M+H]⁺.

### Step 2: 6-bromo-2-(fluoromethyl)-1-methyl-1H-benzo[d]imidazole

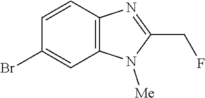 [SMILES: Cn1c(CF)nc2ccc(Br)cc21]

To a solution of (6-bromo-1-methyl-1H-benzo[d]imidazol-2-yl)methanol (200.0 mg; 0.830 mmol; 1.00 eq.) in CH₂Cl₂ (8 mL) was added DAST (267.4 mg; 1.659 mmol; 2.00 eq.) dropwise at 0° C. The resulting mixture was stirred at room temperature for 3 hours under nitrogen atmosphere. The desired product was detected via LCMS. The reaction was quenched with NaHCO₃ aqueous solution at 0° C. The resulting mixture was extracted with CH₂Cl₂ (3×10 mL). The combined organic layers were washed with water (3×10 mL), dried over anhydrous Na₂SO₄, filtered, and concentrated under reduced pressure. The residue was purified by flash chromatography on silica gel column using 15-25% of EtOAc in petroleum ether as eluent to provide 6-bromo-2-(fluoromethyl)-1-methyl-1H-benzo[d]imidazole as an off-white solid (128.0 mg, 63.4%). LCMS (ESI) m/z 243.0, [M+H]⁺.

### Step 3: N-(5-((2-(fluoromethyl)-1-methyl-1H-benzo[d]imidazol-6-yl)ethynyl)-8-(methylamino)-2,7-naphthyridin-3-yl)cyclopropanecarboxamide

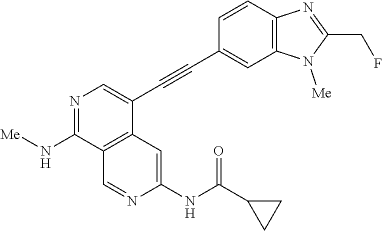 [SMILES: CNc1ncc(C#Cc2ccc3nc(CF)n(C)c3c2)c2cc(NC(=O)C3CC3)ncc12]

N-(5-((2-(fluoromethyl)-1-methyl-1H-benzo[d]imidazol-6-yl)ethynyl)-8-(methylamino)-2,7-naphthyridin-3-yl)cyclopropanecarboxamide was synthesized using a similar procedure that was previously described in example 595, by using NV (5-ethynyl-8-(methylamino)-2,7-naphthyridin-3-yl)cyclopropanecarboxamide and 6-bromo-2-(fluoromethyl)-1-methyl-1H-benzo[d]imidazole as the starting material. LCMS (ESI) m/z 429.2, [M+H]⁺. ¹H NMR (400 MHz, DMSO-d₆) 11.12 (s, 1H), 9.39 (s, 1H), 8.80 (s, 1H), 8.33-8.29 (m, 2H), 8.00 (d, J=1.6 Hz, 1H), 7.73 (d, J=8.0 Hz, 1H), 7.43 (dd, J=8.0, 1.6 Hz, 1H), 5.76 (d, J=47.2 Hz, 2H), 3.93 (s, 3H), 3.03 (d, J=4.4 Hz, 3H), 2.17-2.09 (m, 1H), 0.99-0.79 (m, 4H).

### Example 811: Synthesis of N-(5-((2-ethyl-1-methyl-1H-benzo[d]imidazol-6-yl)ethynyl)-8-(methylamino)-2,7-naphthyridin-3-yl)cyclopropanecarboxamide

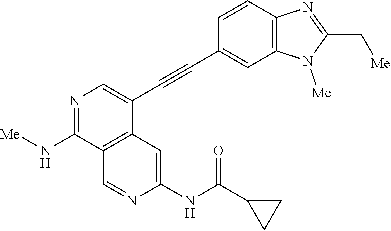 [SMILES: CCc1nc2ccc(C#Cc3cnc(NC)c4cnc(NC(=O)C5CC5)cc34)cc2n1C]

### Step 1: 6-bromo-2-ethyl-1-methyl-1H-benzo[d]imidazole

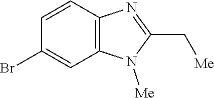 [SMILES: CCc1nc2ccc(Br)cc2n1C]

To a solution of 5-bromo-N¹-methylbenzene-1,2-diamine (500.0 mg; 2.487 mmol; 1.00 eq.) in HCl (3 M in water, 8 mL) was added propionic acid (921.0 mg; 1.2.435 mmol; 5.00 eq.). The resulting mixture was stirred at 100° C. overnight. The desired product was detected via LCMS. The mixture was neutralized to pH=7 with 4 M NaOH aqueous solution. The mixture was extracted with CH₂Cl₂ (3×20 mL). The combined organic layers were washed with brine (3×3 mL), dried over anhydrous Na₂SO₄, and concentrated under vacuum. The residue was purified by flash chromatography on silica gel column using 30-60% of EtOAc in petroleum ether as eluent to provide 6-bromo-2-ethyl-1-methyl-1H-benzo[d]imidazole as a pink solid (494.8 mg, 82.6%). LCMS (ESI) m/z 239.0, [M+H]⁺.

### Step 2: N-(5-((2-ethyl-1-methyl-1H-benzo[d]imidazol-6-yl)ethynyl)-8-(methylamino)-2,7-naphthyridin-3-yl)cyclopropanecarboxamide

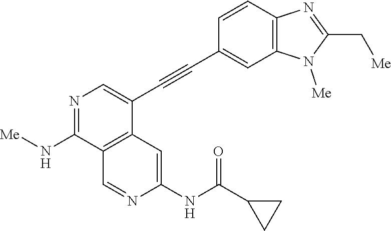 [SMILES: CCc1nc2ccc(C#Cc3cnc(NC)c4cnc(NC(=O)C5CC5)cc34)cc2n1C]

N-(5-((2-ethyl-1-methyl-1H-benzo[d]imidazol-6-yl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 6-bromo-2-ethyl-1-methyl-1H-benzo[d]imidazole as the starting material. LCMS (ESI) m/z 425.2, [M+H]⁺. ¹H NMR (400 MHz, DMSO-d₆) δ 11.12 (s, 1H), 9.38 (s, 1H), 8.79 (s, 1H), 8.32-8.28 (m, 1H), 8.26 (s, 1H), 7.85 (d, J=1.6 Hz, 1H), 7.58 (d, J=8.4 Hz, 1H), 7.33 (dd, J=8.4, 1.6 Hz, 1H), 3.79 (s, 3H), 3.03 (d, J=4.4 Hz, 3H), 2.97-2.82 (m, 2H), 2.12-2.02 (m, 1H), 1.35 (t, J=7.2 Hz, 3H), 0.99-0.73 (m, 4H).

### Example 812: Synthesis of N-(5-((benzo[d][1,3]dioxol-5-yl-2,2-d₂)ethynyl)-8-(methylamino)-2,7-naphthyridin-3-yl)cyclopropanecarboxamide

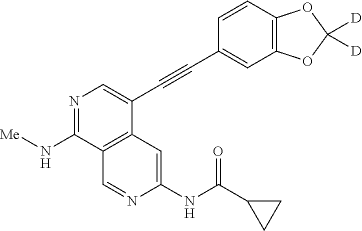 [SMILES: [2H]C1([2H])Oc2ccc(C#Cc3cnc(NC)c4cnc(NC(=O)C5CC5)cc34)cc2O1]

### Step 1: 5-bromobenzo[d][1,3]dioxole-2,2-d₂

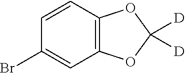 [SMILES: [2H]C1([2H])Oc2ccc(Br)cc2O1]

A solution of 4-bromobenzene-1,2-diol (200.0 mg; 1.058 mmol; 1.00 eq.), Cs₂CO₃ (517.1 mg; 1.587 mmol; 1.50 eq.) and dibromomethane-d₂ (279.1 mg; 1.587 mmol; 1.50 eq.) in DMF (4 mL) was stirred at 110° C. for 1 hour under nitrogen atmosphere. The resulting mixture was diluted with water. (10 mL) and extracted with EtOAc (3×10 mL). The combined organic layers were washed with brine (2×10 mL), dried over anhydrous Na₂SO₄. After filtration, the filtrate was concentrated under reduced pressure. The residue was purified by flash chromatography on silica gel column using 0-15% of EtOAc in petroleum ether as eluent to provide 5-bromobenzo[d][1,3]dioxole-2,2-d₂ as a yellow oil (120.0 mg, 55.8%). ¹H NMR (400 MHz, DMSO-d₆) δ 7.17 (d, J=2.0 Hz, 1H), 7.01 (dd, J=8.4, 2.0 Hz, 1H), 6.89 (d, J=8.4 Hz, 1H).

### Step 2: N-(5-((benzo[d][1,3]dioxol-5-yl-2,2-d₂)ethynyl)-8-(methylamino)-2,7-naphthyridin-3-yl)cyclopropanecarboxamide

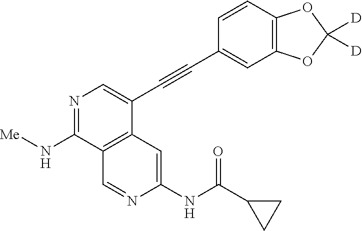 [SMILES: [2H]C1([2H])Oc2ccc(C#Cc3cnc(NC)c4cnc(NC(=O)C5CC5)cc34)cc2O1]

N-(5-((benzo[d][1,3]dioxol-5-yl-2,2-d₂)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 5-bromobenzo[d][1,3]dioxole-2,2-d₂ as the starting material. LCMS (ESI) m/z 389.2, [M+H]⁺. ¹H NMR (400 MHz, DMSO-d₆) δ 11.07 (s, 1H), 9.37 (s, 1H), 8.64 (s, 1H), 8.45-8.25 (m, 1H), 8.24 (s, 1H), 7.12-7.06 (m, 2H), 6.99 (d, J=8.4 Hz, 1H), 3.02 (d, J=4.4 Hz, 3H), 2.20-2.00 (m, 1H), 0.98-0.79 (m, 4H).

### Example 813: Synthesis of N-(8-(methylamino)-5-((2-methylbenzofuran-5-yl)ethynyl)-2,7-naphthyridin-3-yl)cyclopropanecarboxamide

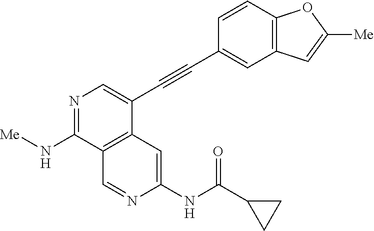 [SMILES: CNc1ncc(C#Cc2ccc3oc(C)cc3c2)c2cc(NC(=O)C3CC3)ncc12]

### Step 1: 2-(4-bromo-2-formylphenoxy)propanoic acid

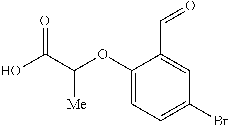 [SMILES: CC(Oc1ccc(Br)cc1C=O)C(=O)O]

A mixture of 5-bromo-2-hydroxybenzaldehyde (1.00 g; 4.975 mmol; 1.00 eq.), ethyl 2-bromopropanoate (2.70 g; 14.925 mmol; 3.00 eq.) and K₂CO₃ (2.06 g; 1.4.925 mmol; 3.00 eq.) in acetonitrile (30 mL) was stirred at room temperature for 12 hours. The reaction mixture was concentrated to dryness under vacuum. The crude material was dissolved in a mixture solvent of MeOH/water (3:1, 20 mL) and stirred at 50° C. for 2 hours. The reaction was cooled to room temperature and acidified to pH=3 with 1 M HCl aqueous solution. After MeOH was removed under reduced pressure, the aqueous suspension was diluted with water (20 mL) and extracted with dichloromethane (3×100 mL). The combined organic layers were washed with brine (3×10 mL), dried over anhydrous Na₂SO₄. After filtration, the filtrate was concentrated under reduced pressure to afford 2-(4-bromo-2-formylphenoxy)propanoic acid as a beige solid (1.2 g, 83.9%). LCMS (ESI) m/z 273.1, [M+H]⁺.

### Step 2: 5-bromo-2-methylbenzofuran

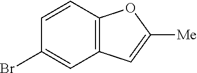 [SMILES: Cc1cc2cc(Br)ccc2o1]

A mixture of 2-(4-bromo-2-formyi.phenoxy)propanoic acid (1.39 g; 5.086 mmol; 1.00 eq.) and sodium acetate solution (1.25 g; 15.260 mmol; 3.00 eq.) in acetic anhydride (6 mL) was heated to reflux and stirred overnight. The reaction was allowed to cool down to room temperature and diluted with toluene (10 mL). The resulting mixture was treated with 1 M NaOH aqueous solution (10 mL) and stirred at room temperature for 20 minutes. The solution was further diluted with water (50 mL) and extracted with EtOAc (3×20 mL). The combined organic layers were washed with brine (3×10 mL), and dried over anhydrous Na₂SO₄. After filtration, the filtrate was concentrated under reduced pressure. The residue was purified by flash chromatography on silica gel column using 0-10% of EtOAc in petroleum ether to afford 5-bromo-2-methylbenzofuran as a colorless oil (700.0 mg, 62.0%). LCMS (ESI) m/z 211.1, [M+H]⁺.

### Step 3: N-(8-(methylamino)-5-((2-methylbenzofuran-5-yl)ethynyl)-2,7-naphthyridin-3-yl)cyclopropanecarboxamide

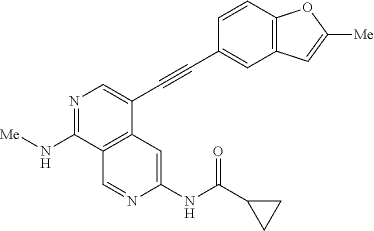 [SMILES: CNc1ncc(C#Cc2ccc3oc(C)cc3c2)c2cc(NC(=O)C3CC3)ncc12]

N-(8-(methylamino)-5-((2-methylbenzofuran-5-yl)ethynyl)-2,7-naphthyridin-3-yl)cyclopropanecarboxamide as synthesized using a similar procedure that was previously described in example 595, by using N-(5-ethynyl-8-(methylamino)-2,7-naphthyridin-3-yl)cyclopropanecarboxamide and 5-bromo-2-methylbenzofuran as the starting material. LCMS (ESI) m/z 397.2, [M+H]⁺. ¹H NMR (400 MHz, DMSO-d₆) δ 11.08 (s, 1H), 9.38 (s, 1H), 8.68 (s, 1H), 8.49-8.21 (m, 2H), 7.74 (d, J=1.6 Hz, 1H), 7.56 (d, J=8.4 Hz, 1H), 7.41 (dd, J=8.4, 1.6 Hz, 1H), 6.63 (s, 1H), 3.02 (d, J=4.4 Hz, 3H), 2.47 (s, 3H), 2.16-2.02 (m, 1H), 0.94-0.80 (m, 4H).

### Example 814: Synthesis of (S)—N-(8-(methylamino)-5-((4-((2-methylmorpholino)methyl)phenyl)ethynyl)-2,7-naphthyridin-3-yl)cyclopropanecarboxamide

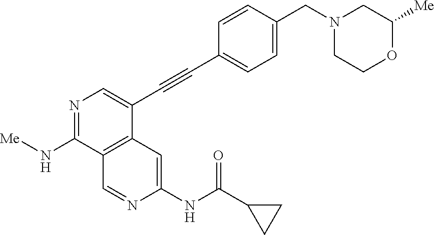 [SMILES: CNc1ncc(C#Cc2ccc(CN3CCO[C@@H](C)C3)cc2)c2cc(NC(=O)C3CC3)ncc12]

### Step 1: (S)-4-(4-bromobenzyl)-2-methylmorpholine

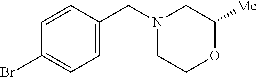 [SMILES: C[C@H]1CN(Cc2ccc(Br)cc2)CCO1]

A solution of 1-bromo-4-(bromomethyl)benzene (200.0 mg; 0.800 mmol; 1.00 eq.), (2S)-2-methylmorpholine (121.4 mg; 1.200 mmol; 1.50 eq.) and K₂CO₃ (331.7 mg; 2.400 mmol; 3.00 eq.) in acetonitrile (4 mL) was stirred at 60° C. for 4 hours. The desired product was detected via LCMS. The resulting mixture was concentrated under reduced pressure. The residue was purified by flash chromatography on silica gel column using 30-40% of EtOAc in petroleum ether as eluent to provide (S)-4-(4-bromobenzyl)-2-methylmorpholine as a colorless oil (212.8 mg, 98.4%). LCMS (ESI) m/z 270.0, [M+H]⁺.

### Step 2: (S)—N-(8-(methylamino)-5-((4-((2-methylmorpholino)methyl)phenyl)ethynyl)-2,7-naphthyridin-3-yl)cyclopropanecarboxamide

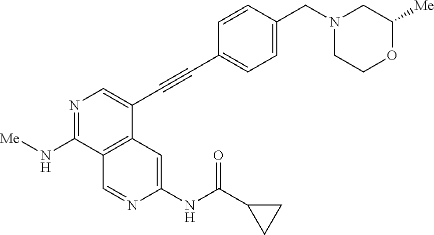 [SMILES: CNc1ncc(C#Cc2ccc(CN3CCO[C@@H](C)C3)cc2)c2cc(NC(=O)C3CC3)ncc12]

(S)—N-(8-(methylamino)-5-44-((2-methylmorpholino)methyl)phenyl)ethynyl)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and (S)-4-(4-bromobenzyl)-2-methylmorpholine as the starting material. LCMS (ESI) m/z 456.2, [M+H]⁺. ¹H NMR (400 MHz, DMSO-d₆) δ 11.07 (s, 1H), 9.37-0.14), 8.65 (s, 0.14), 8.40-8.30 (m, 1H), 8.27 (s, 1H), 7.52 (d, J=8.0 Hz, 2H), 7.37 (d, J=8.0 Hz, 2H), 3.80-3.73 (m, 1H), 3.55-3.48 (m, 4H), 3.02 (d, J=4.4 Hz, 3H), 2.71-2.66 (m, 1H), 2.66-2.59 (m, 1H), 2.16-2.01 (m, 2H), 1.83-1.72 (m, 1H), 1.04 (d, J=6.4 Hz, 3H), 0.91-0.80 (m, 4H).

### Example 815: Synthesis of N-(5-((4-(3-oxa-8-aza-bicyclo[3.2.1]octan-8-ylmethyl)phenyl)ethynyl)-8-(methylamino)-2,7-naphthyridin-3-yl)cyclopropanecarboxamide

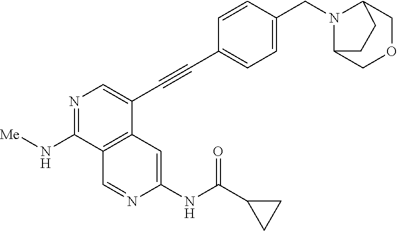 [SMILES: CNc1ncc(C#Cc2ccc(CN3C4CCC3COC4)cc2)c2cc(NC(=O)C3CC3)ncc12]

### Step 1: 8-(4-bromobenzyl)-3-oxa-8-azabicyclo[3.2.1]octane

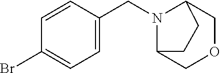 [SMILES: Brc1ccc(CN2C3CCC2COC3)cc1]

A solution of 3-oxa-8-azabicyclo[3.2.1]octane hydrochloride (119.7 mg; 0.800 mmol; 1.00 eq.), 1-bromo-4-(bromomethyl)benzene (200.0 mg; 0.800 mmol; 1.00 eq.) and K₂CO₃ (442.3 mg; 3.200 mmol; 4.00 eq.) in MeCN (10 mL) was stirred at 60° C. for 14 hours. The desired product was detected via LCMS. The resulting mixture was diluted with water (10 mL) and extracted with EtOAc (3×10 mL). The combined organic layers were washed with brine (3×10 mL), and dried over anhydrous Na₂SO₄. After filtration, the filtrate was concentrated under reduced pressure. The residue was purified by flash chromatography on silica gel column using 25-50% of EtOAc in petroleum ether as eluent to provide 8-(4-bromobenzyl)-3-oxa-8-azabicyclo[3.2.1]octane as a off-white oil (148.0 mg, 65.5%). LCMS (ESI) m/z 282.0, [M+H]⁺.

### Step 2: N-(5-((4-(3-oxa-8-aza-bicyclo[3.2.1]octan-8-ylmethyl)phenyl)ethynyl)-8-(methylamino)-2,7-naphthyridin-3-yl)cyclopropanecarboxamide

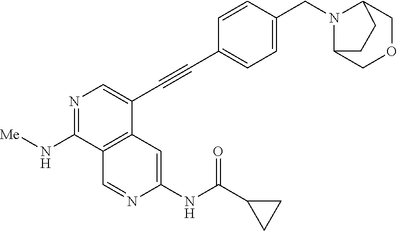 [SMILES: CNc1ncc(C#Cc2ccc(CN3C4CCC3COC4)cc2)c2cc(NC(=O)C3CC3)ncc12]

N-(5-((4-(3-oxa-8-aza-bicyclo[3.2.1]octan-8-ylmethyl)phenyl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 8-(4-bromobenzyl)-3-oxa-8-azabicyclo[3.2.1]octane as the starting material. LCMS (ESI) m/z 468.2, [M+H]⁺. ¹H NMR (400 MHz, DMSO-d₆) δ 11.08 (s, 1H), 9.38 (s, 1H), 8.66 (s, 1H), 8.32-8.30 (m, 1H), 8.27 (s, 1H), 7.52 (d, J=8.4 Hz, 2H), 7.45 (d, J=8.4 Hz, 2H), 3.57 (d, J=10.0 Hz, 2H), 3.52-3.40 (m, 4H), 3.10-2.93 (m, 5H), 2.14-2.05 (m, 1H), 2.02-1.90 (m, 2H), 1.84-1.72 (m, 2H), 0.98-0.76 (m, 4H).

### Example 816: Synthesis of N-(5-((4-((3-methoxyazetidin-1-yl)methyl)phenyl)ethynyl)-8-(methylamino)-2,7-naphthyridin-3-yl)cyclopropanecarboxamide

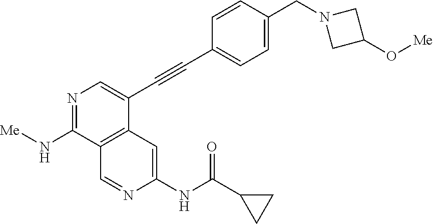 [SMILES: CNc1ncc(C#Cc2ccc(CN3CC(OC)C3)cc2)c2cc(NC(=O)C3CC3)ncc12]

### Step 1: 1-(4-bromobenzyl)-3-methoxyazetidine

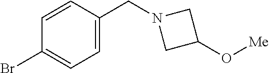 [SMILES: COC1CN(Cc2ccc(Br)cc2)C1]

A mixture of 1-bromo-4-(bromomethyl)benzene (200.0 mg; 0.800 mmol; 1.00 eq.), 3-methoxyazetidine hydrochloride (99.2 mg; 0.800 mmol; 1.00 eq.) and K₂CO₃ (331.8 mg; 2.401 mmol; 3.00 eq.) in acetonitrile (3 mL) was stirred at 60° C. for 14 hours under nitrogen atmosphere. The desired product was detected via LCMS. The mixture was purified by flash chromatography on pre-packed C18 column using 30-50% of MeCN in water (10 mmol/L NH₄HCO₃) as eluent to provide 1-(4-bromobenzyl)-3-methoxyazetidine as a colorless liquid (120.0 mg, 55.6%). LCMS (ESI) m/z 256.0, [M+H]⁺.

### Step 2: N-(5-((4-((3-methoxyazetidin-1-yl)methyl)phenyl)ethynyl)-8-(methylamino)-2,7-naphthyridin-3-yl)cyclopropanecarboxamide

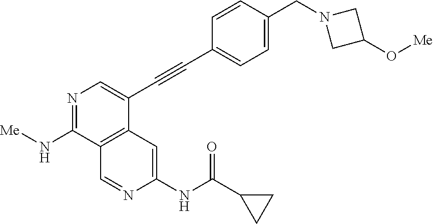 [SMILES: CNc1ncc(C#Cc2ccc(CN3CC(OC)C3)cc2)c2cc(NC(=O)C3CC3)ncc12]

N-(5-((4-((3-methoxyazetidin-1-yl)methyl)phenyl)ethynyl)-8-(methylamino)-2,7-naphthyridin-3-yl)cyclopropanecarboxamide was synthesized using a similar procedure that was previously described in example 595, by using N-(5-ethynyl-8-(methylamino)-2,7-naphthyridin-3-yl)cyclopropanecarboxamide and 1-(4-bromobenzyl)-3-methoxyazetidine as the starting material. LCMS (ESI) m/z 442.2, [M+H]⁺. ¹H NMR (400 MHz, DMSO-d₆) δ 11.08 (s, 1H), 9.37 (s, 1H), 8.65 (s, 1H), 8.41-8.30 (m, 1H), 8.27 (s, 1H), 7.50 (d, J=8.0 Hz, 2H), 7.33 (d, J=8.0 Hz, 2H), 4.06-3.85 (m, 1H), 3.61 (s, 2H), 3.53-3.40 (m, 2H), 3.16 (s, 3H), 3.02 (d, J=4.4 Hz, 3H), 2.92-2.73 (m, 2H), 2.18-2.00 (m, 1H), 0.94-0.80 (m, 4H).

### Example 817: Synthesis of N-(5-((1-(methyl-d₃)-1H-benzo[d][1,2,3]triazol-6-yl)ethynyl)-8-(methylamino)-2,7-naphthyridin-3-yl)cyclopropanecarboxamide

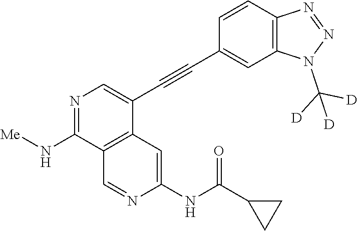 [SMILES: [2H]C([2H])([2H])n1nnc2ccc(C#Cc3cnc(NC)c4cnc(NC(=O)C5CC5)cc34)cc21]

### Step 1: 5-bromo-N-(methyl-d₃)-2-nitroaniline

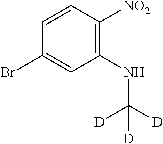 [SMILES: [2H]C([2H])([2H])Nc1cc(Br)ccc1[N+](=O)[O-]]

To a stirred solution of methan-d₃-amine hydrochloride (175.8 mg; 2.493 mmol; 1.10 eq.) and K₂CO₃ (945.6 mg; 6.842 mmol; 3.00 eq.) in DMF (5 mL) was added 4-bromo-2-fluoro-1-nitrobenzene (500.2 mg; 2.273 mmol; 1.00 eq.). The resulting mixture was stirred at room temperature for 14 hours. The desired product was detected via LCMS. The reaction was diluted with water (50 mL) to precipitate solids. The precipitated solids were collected by filtration and washed with water (2×5 mL). The solids were dried under vacuum to afford 5-bromo-N-(methyl-d₃)-2-nitroaniline as a white solid (520.0 mg, 97.7%). LCMS (ESI) m/z 234.0, [M+H]⁺.

### Step 2: 5-bromo N¹-(methyl-d₃)benzene-1,2-diamine

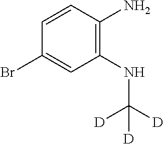 [SMILES: [2H]C([2H])([2H])Nc1cc(Br)ccc1N]

A solution of 5-bromo-N-(methyl-d₃)-2-nitroaniline (500.0 mg; 2.136 mmol; 1.00 eq.) and Fe powder (1.20 g; 21.488 mmol; 10.06 eq.) in AcOH (5 mL) was stirred at 70° C. for 3 hours under nitrogen atmosphere. The desired product was detected via LCMS. The resulting mixture was diluted with water (30 mL) and neutralized to pH=7 with Na₂CO₃ aqueous solution. The aqueous phase was extracted with EtOAc (3×50 mL). The combined organic layers were washed with brine (3×50 mL) and dried over anhydrous Na₂SO₄. After filtration, the filtrate was concentrated under reduced pressure. The residue was purified by flash chromatography on silica gel column using 10-30% of EtOAc in petroleum ether as eluent to provides-bromo-N¹-(methyl-d₃)benzene-1,2-diamine as a brown oil (330.2 mg, 75.7%). LCMS (ESI) m/z 204.0, [M+H]⁺.

### Step 3: 6-bromo-1-(methyl-d₃)-1H-benzo[d][1,2,3]triazole

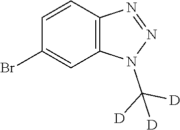 [SMILES: [2H]C([2H])([2H])n1nnc2ccc(Br)cc21]

To a solution of 5-bromo-N¹-(methyl-d₃)benzene-1,2-diamine (150.2 mg; 0.735 mmol; 1.00 eq.) in HCl (12 M in water, 2 mL) was added a solution of NaNO₂ (73.1 mg; 1.058 mmol; 1.48 eq.) in water (0.5 mL) at 0° C. The resulting mixture was stirred at room temperature for 1 hour under nitrogen atmosphere. The desired product was detected via LCMS. The resulting mixture was diluted with water (30 mL) and neutralized to pH=7 with 4 M of NaOH aqueous solution. The precipitated solids were collected by filtration and washed with water (2×5 mL). The solids were dried under vacuum to afford 6-bromo-1-(methyl-d₃)-1H-benzo[d][1,2,3]triazole as a white solid (140.2 mg, 88.5%). LCMS (ESI) m/z 215.0, [M+H]⁺.

### Step 4: N-(5-((1-(methyl-d₃)-1H-benzo[d][1,2,3]triazol-6-yl)ethynyl)-8-(methylamino)-2,7-naphthyridin-3-yl)cyclopropanecarboxamide

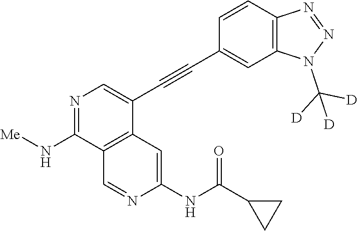 [SMILES: [2H]C([2H])([2H])n1nnc2ccc(C#Cc3cnc(NC)c4cnc(NC(=O)C5CC5)cc34)cc21]

N-(5-((1-(methyl-d₃)-1H-benzo[d][1,2,3]triazol-6-yl)ethynyl)-8-(methylamino)-2,7-naphthyridin-3-yl)cyclopropanecarboxamide was synthesized using a similar procedure that was previously described in example 595, by using NV (5-ethynyl-8-(methylamino)-2,7-naphthyridin-3-yl)cyclopropanecarboxamide and 6-bromo-1-(methyl-d₃)-1H-benzo[d][1,2,3]triazole as the starting material. LCMS (ESI) m/z 401.2, [M+H]⁺. ¹H NMR (300 MHz, DMSO-d₆) δ 11.12 (s, 1H), 9.40 (s, 1H), 8.78 (s, 1H), 8.42-8.35 (m, 1H), 8.33 (s, 1H), 8.18 (d, J=1.6 Hz, 1H), 8.10 (d, J=11.6 Hz, 1H), 7.60-7.42 (m, 1H), 3.04 (d, J=4.4 Hz, 3H), 2.18-2.04 (m, 1H), 0.97-0.77 (m, 4H).

### Example 818: Synthesis of N-(5-((2-(difluoromethyl)-1-methyl-1H-benzo[d]imidazol-6-yl)ethynyl)-8-((methyl-d₃)amino)-2,7-naphthyridin-3-yl)cyclopropanecarboxamide

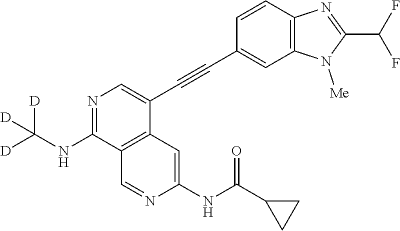 [SMILES: [2H]C([2H])([2H])Nc1ncc(C#Cc2ccc3nc(C(F)F)n(C)c3c2)c2cc(NC(=O)C3CC3)ncc12]

### Step 1: N-(8-((methyl-d₃)amino)-5-((trimethylsilyl)ethynyl)-2,7-naphthyridin-3-yl)cyclopropanecarboxamide

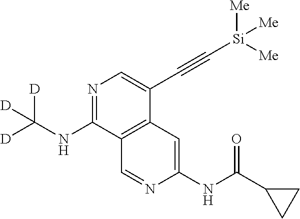 [SMILES: [2H]C([2H])([2H])Nc1ncc(C#C[Si](C)(C)C)c2cc(NC(=O)C3CC3)ncc12]

A solution of N-(5-bromo-8-((methyl-d₃)amino)-2,7-naphthyridin-3-yl)cyclopropanecarboxamide (1.00 g; 3.085 mmol; 1.00 eq.), ethynyltrimethylsilane (605.9 mg; 6.170 mmol; 2.00 eq.), Pd(dppf)Cl₂·CH₂Cl₂ (1.26 g; 1.542 mmol; 0.50 eq.), CuI (293.7 mg; 1.542 mmol; 0.50 eq.) and DIEA (2.39 g; 18.510 mmol; 6.00 eq.) in DMF (10 mL) was stirred at 50° C. for 2 hours under nitrogen atmosphere. The desired product was detected via LCMS. The mixture was diluted with water (50 mL) and extracted with EtOAc (3×50 mL). The combined organic layers were washed with brine (3×20 mL), dried over anhydrous Na₂SO₄. After filtration, the filtrate was concentrated under reduced pressure. The residue was purified by flash chromatography on silica gel column using 0-5% of MeOH in CH₂Cl₂ as eluent to provide N-(8-((methyl-d₃)amino)-5-((trimethyl silyl)ethynyl)-2,7-naphthyridin-3-yl)cyclopropanecarboxamide as a yellow solid (931 mg, 84.5%). LCMS (ESI) m/z 342.2, [M+H]⁺.

### Step 2: N-(5-ethynyl-8-((methyl-d₃)amino)-2,7-naphthyridin-3-yl)cyclopropanecarboxamide

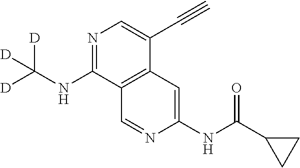 [SMILES: [2H]C([2H])([2H])Nc1ncc(C#C)c2cc(NC(=O)C3CC3)ncc12]

A solution of N-(8-((methyl-d₃)amino)-5-((trimethylsilyl)ethynyl)-2,7-naphthyridin-3-yl)cyclopropanecarboxamide (920.0 mg; 2.694 mmol; 1.00 eq.) and K₂CO₃ (994.0 mg; 7.1.93 mmol; 2.67 eq.) in MeOH (10 mL) was stirred at room temperature for 1 hour. The desired product was detected via LCMS. The resulting mixture was concentrated under vacuum. The residue was purified by flash chromatography on silica gel column using 0-4% of MeOH in CH₂Cl₂ as eluent to provide N-(5-ethynyl-8-((methyl-d₃)amino)-2,7-naphthyridin-3-yl)cyclopropanecarboxamide as a yellow solid (416.4 mg, 56.5%). LCMS (ESI) m/z 270.1, [M+H]⁺.

### Step 3: N-(5-((2-(difluoromethyl)-1-methyl-1H-benzo[d]imidazol-6-yl)ethynyl)-8-((methyl-d₃)amino)-2,7-naphthyridin-3-yl)cydopropanecarboxamide

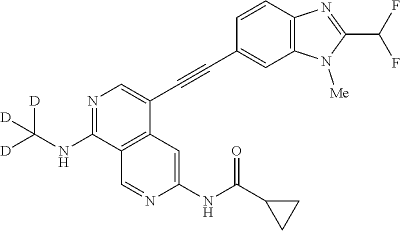 [SMILES: [2H]C([2H])([2H])Nc1ncc(C#Cc2ccc3nc(C(F)F)n(C)c3c2)c2cc(NC(=O)C3CC3)ncc12]

N-(5-((2-(difluoromethyl)-1-methyl-1H-benzo[d]imidazol-6-yl)ethynyl)-8-((methyl-d₃)amino)-2,7-naphthyridin-3-yl)cyclopropanecarboxamide was synthesized using a similar procedure that was previously described in Example 595, by using N-(5-ethynyl-8-((methyl-d₃)amino)-2,7-naphthyridin-3-yl)cyclopropanecarboxamide and 6-bromo-2-(difluoromethyl)-1-methyl-1H-benzo[d]imidazole (Example 739, step 1) as the starting material. LCMS (ESI) m/z 450.2, [M+H]⁺. ¹H NMR (400 MHz, DMSO-d₆) δ 11.13 (s, 1H), 9.39 (s, 1H), 8.79 (s, 1H), 8.37-8.28 (m, 2H), 8.06 (s, 1H), 7.81 (d, J=8.4 Hz, 1H), 7.58-7.29 (m, 2H), 3.99 (s, 3H), 2.17-2.07 (m, 1H), 0.92-0.83 (m, 4H).

### Example 819: Synthesis of (1S,2R)—N-(5-((2-(difluoromethyl)-1-methyl-1H-benzo[d]imidazol-6-yl)ethynyl)-8-((methyl-d₃)amino)-2,7-naphthyridin-3-yl)-2-methylcyclopropane-1-carboxamide

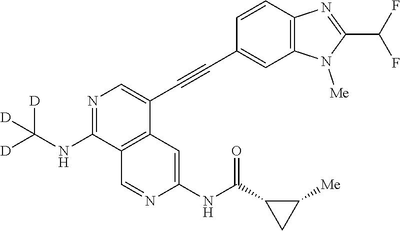 [SMILES: [2H]C([2H])([2H])Nc1ncc(C#Cc2ccc3nc(C(F)F)n(C)c3c2)c2cc(NC(=O)[C@H]3C[C@H]3C)ncc12]

### Step 1: 4-((2-(difluoromethyl)-1-methyl-1H-benzo[d]imidazol-6-yl)ethynyl)-N¹-(methyl-d₃)-2,7-naphthyridine-1,6-diamine

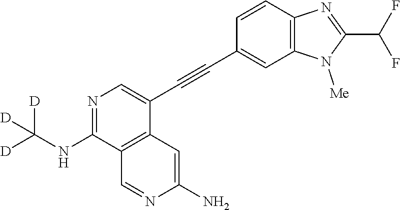 [SMILES: [2H]C([2H])([2H])Nc1ncc(C#Cc2ccc3nc(C(F)F)n(C)c3c2)c2cc(N)ncc12]

To a solution of N-(5-((2-(difluoromethyl)-1-methyl-1H-benzo[d]imidazol-6-yl)ethynyl)-8-((methyl-d₃)amino)-2,7-naphthyridin-3-yl)cyclopropanecarboxamide (Example 818) (180.0 mg; 0.400 mmol; 1.00 eq.) in a mixture solvent of DMSO/MeOH (1:5, 12 mL) was added a solution of NaOH (160.1 mg; 4.000 mmol; 10.00 eq.) in water (3 mL). The mixture was stirred at 70° C. overnight. The desired product was detected via LCMS. The reaction was allowed to cool down to room temperature. The precipitated solids were collected by filtration and washed with MeOH (2×2 mL) to provide 4-((2-(difluoromethyl)-1-methyl-1H-benzo[d]imidazol-6-yl)ethynyl)-N¹-(methyl-d₃)-2,7-naphthyridine-1,6-diamine as a yellow solid (143.6 mg, 93.3%). LCMS (ESI) m/z 382.2, [M+H]⁺.

### Step 2: (1S,2R)—N-(5-((2-(difluoromethyl)-1-methyl-1H-benzo[d]imidazol-6-yl)ethynyl)-8-((methyl-d₃)amino)-2,7-naphthyridin-3-yl)-2-methylcyclopropane-1-carboxamide

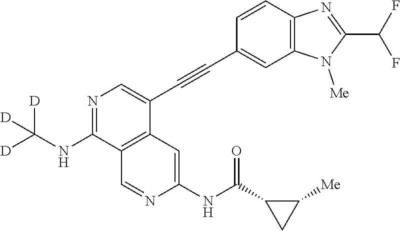 [SMILES: [2H]C([2H])([2H])Nc1ncc(C#Cc2ccc3nc(C(F)F)n(C)c3c2)c2cc(NC(=O)[C@H]3C[C@H]3C)ncc12]

To a solution of 4-((2-(difluoromethyl)-1-methyl-1H-benzo[d]imidazol-6-yl)ethynyl)-N¹-(methyl-d₃)-2,7-naphthyridine-1,6-diamine (80.0 mg; 0.211 mmol; 1.00 eq.) and (1S,2R)-2-methylcyclopropane-1-carboxylic acid (18.9 mg; 0.189 mmol; 0.9 eq.) in pyridine (4 mL) was added POCl₃ (96.4 mg; 0.630 mmol; 3.00 eq.) dropwise at 0° C. The resulting mixture was stirred at room temperature for 5 minutes. The desired product was detected via LCMS. The mixture was quenched with ice water (5 mL) and extracted with EtOAc (3×25 mL). The combined organic layers were concentrated under vacuum. The residue was purified by flash chromatography on silica gel column using 0-4% MeOH in CH₂Cl₂ as eluent to provide (1S,2R)—N-(5-((2-(difluoromethyl)-1-methyl-1H-benzo[d]imidazol-6-yl)ethynyl)-8-((methyl-d₃)amino)-2,7-naphthyridin-3-yl)-2-methylcyclopropane-1-carboxamide as a yellow solid (34.4 mg, 34.9%). LCMS (ESI) m/z 464.2, [M+H]⁺. ¹H NMR (400 MHz, DMSO-d₆) δ 11.03 (s, 1H), 9.38 (s, 1H), 8.80 (s, 1H), 8.33-8.29 (m, 2H), 8.04 (s, 1H), 7.80 (d, J=8.4 Hz, 1H), 7.60-7.28 (m, 2H), 3.98 (s, 3H), 2.17-2.09 (m, 1H), 1.38-1.29 (m, 1H), 1.18 (d, J=6.0 Hz, 3H), 1.07-0.99 (m, 1H), 0.91-0.82 (m, 1H).

### Example 820: Synthesis of N-(8-(methylamino)-5-((4-(morpholinomethyl)phenyl)ethynyl)-2,7-naphthyridin-3-yl)cyclopropanecarboxamide

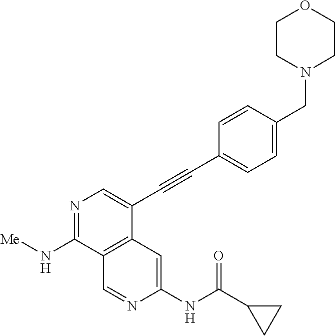 [SMILES: CNc1ncc(C#Cc2ccc(CN3CCOCC3)cc2)c2cc(NC(=O)C3CC3)ncc12]

The compound was synthesized using a similar procedure that was previously described in Example 595 by using N-(5-ethynyl-8-(methylamino)-2,7-naphthyridin-3-yl)cyclopropanecarboxamide and 4-(4-bromobenzyl)morpholine as the starting material. LCMS (ESI) m/z 442.0 [M+H]⁺; ¹H NMR (400 MHz, DMSO-d₆) δ ppm 11.08 (s, 1H), 9.37 (s, 1H), 8.66 (s, 1H), 8.32 (br d, J=4.9 Hz, 1H), 8.27 (s, 1H), 7.52 (d, J=8.0 Hz, 2H), 7.38 (d, J=8.1 Hz, 2H), 3.55-3.66 (m, 4H), 3.50 (s, 2H), 3.02 (d, J=4.3 Hz, 3H), 2.38 (br s, 4H), 2.03-2.13 (m, 1H), 0.81-0.95 (m, 4H).

### Example 821: Synthesis of (1S,2R)-2-methyl-N-(8-(methylamino)-5-((4-(morpholinomethyl)phenyl)ethynyl)-2,7-naphthyridin-3-yl)cyclopropane-1-carboxamide

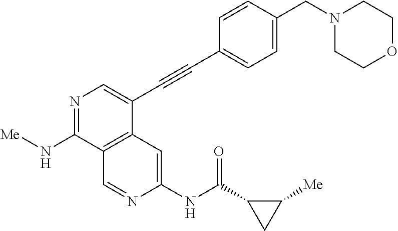 [SMILES: CNc1ncc(C#Cc2ccc(CN3CCOCC3)cc2)c2cc(NC(=O)[C@H]3C[C@H]3C)ncc12]

### Step 1: N-(8-(methylamino)-5-((4-(morpholinomethyl)phenyl)ethynyl)-2,7-naphthyridin-3-yl)cyclopropanecarboxamide (Example 820)

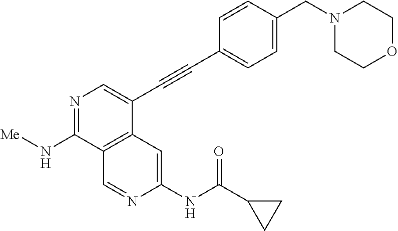 [SMILES: CNc1ncc(C#Cc2ccc(CN3CCOCC3)cc2)c2cc(NC(=O)C3CC3)ncc12]

A solution of 4-(4-bromobenzyl)morpholine (96.0 mg; 0.375 mmol; 1.00 eq.), N-(5-ethynyl-8-(methylamino)-2,7-naphthyridin-3-yl)cyclopropanecarboxamide (150.0 mg; 0.563 mmol; 1.50 eq.), XPhos Pd G₃ (95.5 mg; 0.113 mmol; 0.30 eq.), XPhos (53.7 mg; 0.113 mmol; 0.30 eq.), Et₃N (152.1 mg; 1.503 mmol; 4.01 eq.) and CuI (21.5 mg; 0.113 mmol; 0.30 eq.) in DMF (5 mL) was stirred at 110° C. for 1 hour under nitrogen atmosphere. The desired product was detected via LCMS. The resulting mixture was concentrated under vacuum. The residue was purified by flash chromatography on silica gel column using 5-20% of MeOH in CH₂Cl₂ as eluent to provide N-(8-(methylamino)-5-((4-(morpholinomethyl)phenyl)ethynyl)-2,7-naphthyridin-3-yl)cyclopropanecarboxamide as a yellow solid (158.0 mg, 95.4%). LCMS (ESI) m/z 442.0 [M+H]+; ¹H NMR (400 MHz, DMSO-d₆) δ ppm 11.08 (s, 1H), 9.37 (s, 1H), 8.66 (s, 1H), 8.32 (br d, J=4.9 Hz, 1H), 8.27 (s, 1H), 7.52 (d, J=8.0 Hz, 2H), 7.38 (d, J=8.1 Hz, 2H), 3.55-3.66 (m, 4H), 3.50 (s, 2H), 3.02 (d, J=4.3 Hz, 3H), 2.38 (br s, 4H), 2.03-2.13 (m, 1H), 0.81-0.95 (m, 4H).

### Step 2: N¹-methyl-4-((4-(morpholinomethyl)phenyl)ethynyl)-2,7-naphthyridine-1,6-diamine

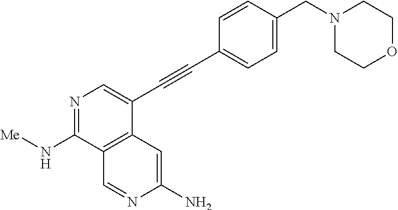 [SMILES: CNc1ncc(C#Cc2ccc(CN3CCOCC3)cc2)c2cc(N)ncc12]

To a solution of N-(8-(methylamino)-5-((4-(morpholinomethyl)phenyl)ethynyl)-2,7-naphthyridin-3-yl)cyclopropanecarboxamide (158.0 mg; 0.358 mmol; 1.00 eq.) in a mixture solvent of DMSO/MeOH (1/5, 12 mL) was added a solution of NaOH (143.3 mg; 3.583 mmol; 10.01 eq.) in water (3 mL) at room temperature. The resulting solution was stirred at 60° C. for 16 hours. The desired product was detected via LCMS. The MeOH was removed under vacuum to precipitate solids. The precipitated solids were collected by filtration and washed with water. The solids were dried under reduced pressure to afford N¹-methyl-4-((4-(morpholinomethyl)phenyl)ethynyl)-2,7-naphthyridine-1,6-diamine as a yellow solid (100.0 mg, 74.8%). LCMS (ESI) m/z 374.2, [M+H]⁺.

### Step 3: (1S,2R)-2-methyl-N-(8-(methylamino)-5-((4-(morpholinomethyl)phenyl)ethynyl)-2,7-naphthyridin-3-yl)cyclopropane-1-carboxamide

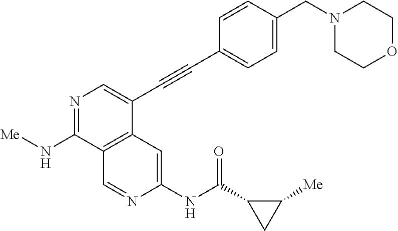 [SMILES: CNc1ncc(C#Cc2ccc(CN3CCOCC3)cc2)c2cc(NC(=O)[C@H]3C[C@H]3C)ncc12]

To a solution of N¹-methyl-4-((4-(morpholinomethyl)phenyl)ethynyl)-2,7-naphthyridine-1,6-1191 diamine (100.0 mg; 0.268 mmol; 1.00 eq.) and (1S,2R)-2-methylcyclopropane-1-carboxylic acid (24.0 mg; 0.240 mmol; 0.90 eq.) in pyridine (3 mL) was added POCl₃ (123.0 mg; 0.802 mmol; 3.00 eq.) at 0° C. The reaction was stirred at room temperature for 0.5 hour under nitrogen atmosphere. The desired product was detected via LCMS. The reaction was quenched with water (5 mL) and extracted with EtOAc (2×50 mL). The combined organic layers were washed with brine (3×10 mL), dried over anhydrous Na₂SO₄. After filtration, the filtrate was concentrated under reduced pressure. The residue was purified by flash chromatography on silica gel column using 2-10% of MeOH in CH₂Cl₂ as eluent to the crude product. The crude product was purified by flash chromatography on pre-packed C18 column using 20-80% of THF/MeCN=1:3 in water (10 mmol/L NH₄HCO₃) as eluent to provide (1S,2R)-2-methyl-N-(8-(methylamino)-5-((4-(morpholinomethyl)phenyl)ethynyl)-2,7-naphthyridin-3-yl)cyclopropane-1-carboxamide as a yellow solid (54.2 mg, 44.4%). LCMS (ESI) m/z 456.2, [M+H]⁺. ¹H NMR (400 MHz, DMSO-d₆) δ 10.98 (s, 1H), 9.37 (s, 1H), 8.68 (s, 1H), 8.42-8.29 (m, 1H), 8.27 (s, 1H), 7.60-7.52 (m, 2H), 7.42-7.35 (m, 2H), 3.67-3.54 (m, 4H), 3.50 (s, 2H), 3.02 (d, J=4.4 Hz, 3H), 2.46-2.29 (m, 4H), 2.16-2.04 (m, 1H), 1.42-1.25 (m, 1H), 1.16 (d, J=6.8 Hz, 3H), 1.07-0.97 (m, 1H), 0.91-0.80 (m, 1H).

### Examples 822 and 823: Synthesis of (1S,2R)-2-(fluoromethyl)-N-(5-((5-methoxypyridin-2-yl)ethynyl)-8-(methylamino)-2,7-naphthyridin-3-yl)cyclopropane-1-carboxamide (Example 822) and (1R,2S)-2-(fluoromethyl)-N-(5-((5-methoxypyridin-2-yl)ethynyl)-8-(methylamino)-2,7-naphthyridin-3-yl)cyclopropane-1-carboxamide (Example 823)

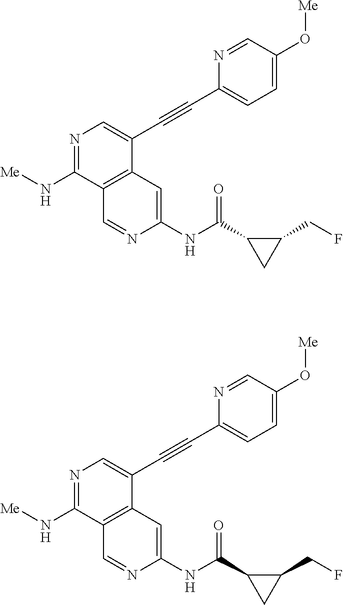 [SMILES: CNc1ncc(C#Cc2ccc(OC)cn2)c2cc(NC(=O)[C@H]3C[C@H]3CF)ncc12 ; CNc1ncc(C#Cc2ccc(OC)cn2)c2cc(NC(=O)[C@@H]3C[C@@H]3CF)ncc12]

To a solution of 4-((5-methoxypyridin-2-yl)ethynyl)-N¹-methyl-2,7-naphthyridine-1,6-diamine (Example 742, step 2) (120.0 mg; 0.393 mmol; 1.00 eq.) and 2-(fluoromethyl)cyclopropane-1-carboxylic acid (cis racemate) (41.7 mg; 0.353 mmol; 0.90 eq.) in pyridine (5 mL) was added POCl₃ (180.7 mg; 1.179 mmol; 3.00 eq.) dropwise at 0° C. The resulting mixture was stirred at room temperature for 1 hour. The desired product was detected via LCMS. The solution was quenched with water (2 mL) and extracted with EtOAc (3×20 mL). The combined organic layers were washed with brine (10 mL) just once and dried over anhydrous Na₂SO₄. After filtration, the filtrate was concentrated under reduced pressure. The residue was purified by flash chromatography on silica gel column using 5-10% of MeOH in CH₂Cl₂ as eluent to afford the cis racemate. The cis racemate was separated by Prep-Chiral-HPLC (Column: CHIRALPAK IG, 2×25 cm, 5 μm; Mobile Phase A: Hex (0.5% 2 M NH₃-MeOH), Mobile Phase B: EtOH:DCM=1:1; Flow rate: 20 mL/min; Gradient: 70% B to 70% B in 10 min; Wave Length: 220/254 nm; R_{T1}(min): 5.98; R_{T2}(min): 8.63) to afford (1S,2R)-2-(fluoromethyl)-N-(5-((5-methoxypyridin-2-yl)ethynyl)-8-(methylamino)-2,7-naphthyridin-3-yl)cyclopropane-1-carboxamide (Example 822, the faster peak) as a yellow solid (16.8 mg, 9.9%) and (1R,2S)-2-(fluoromethyl)-N-(5-((5-methoxypyridin-2-yl)ethynyl)-8-(methylamino)-2,7-naphthyridin-3-yl)cyclopropane-1-carboxamide (Example 823, the slower peak) as a yellow solid (20.5 mg, 12.7%). The two configurations are arbitrarily assigned. LCMS (ESI) m/z 406.2, [M+H]⁺.

HNMR for Example 822: ¹H NMR (400 MHz, DMSO-d₆) δ 11.16 (s, 1H), 9.39 (s, 1H), 8.61 (s, 1H), 8.47-8.25 (m, 3H), 7.62 (d, J=8.8 Hz, 1H), 7.48 (dd, J=8.8, 3.2 Hz, 1H), 4.90-4.65 (m, 1H), 4.65-4.40 (m, 1H), 3.89 (s, 3H), 3.03 (d, J=4.4 Hz, 3H) 2.38-2.28 (m, 1H), 1.83-1.71 (m, 1H), 1.27-1.16 (m, 1H), 1.16-1.03 (m, 1H).

HNMR for Example 823: ¹H NMR (400 MHz, DMSO-d₆) δ 11.16 (s, 1H), 9.39 (s, 1H), 8.61 (s, 1H), 8.47-8.25 (m, 3H), 7.62 (d, J=8.8 Hz, 1H), 7.48 (dd, J=8.8, 3.2 Hz, 1H), 4.90-4.65 (m, 1H), 4.65-4.40 (m, 1H), 3.89 (s, 3H), 3.03 (d, J=4.4 Hz, 3H), 2.38-2.28 (m, 1H), 1.83-1.71 (m, 1H), 1.27-1.16 (m, 1H), 1.16-1.03 (m, 1H)

### Example 824: Synthesis of 9-bromo-N-methyl-3H-pyrrolo[2,3-c][2,7]naphthyridin-6-amine

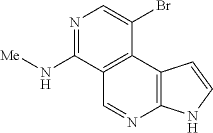 [SMILES: CNc1ncc(Br)c2c1cnc1[nH]ccc12]

### Step 1: 6-chloro-N-(4-methoxybenzyl)-N-methyl-2,7-naphthyridin-1-amine

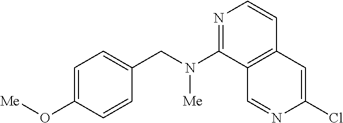 [SMILES: COc1ccc(CN(C)c2nccc3cc(Cl)ncc23)cc1]

To a solution of 6-chloro-2,7-naphthyridin-1(2H)-one (13.00 g; 71.98 mmol; 1.00 eq.) and PyBOP (75.00 g; 144.12 mmol; 2.00 eq.) in DMA (30 mL) were added 1-(4-methoxyphenyl) N methylmethanamine (25.00 g; 165.33 mmol; 2.03 eq.) and DIPEA (46.00 g; 0.356 mmol; 4.94 eq.) at room temperature and stirred at 80° C. for 16 hours under nitrogen atmosphere. The desired product was detected via LCMS. The mixture was allowed to cool down to room temperature. The resulting mixture was diluted with brine (200 mL) and extracted with EtOAc (2×100 mL). The organic layer was washed with brine (2×50 mL), dried over anhydrous Na₂SO₄, filtered, and concentrated under reduced pressure. The residue was purified by flash chromatography on silica gel column using 2-6% of methanol in dichloromethane as eluent to afford 6-chloro-N-(4-methoxybenzyl)-N-methyl-2,7-naphthyridin-1-amine as a brown solid (15.00 g, 66.9%). LCMS (ESI) m/z 314.1, [M+H]⁺.

### Step 2: 4-bromo-6-chloro-N-(4-methoxybenzyl)-N-methyl-2,7-naphthyridin-1-amine

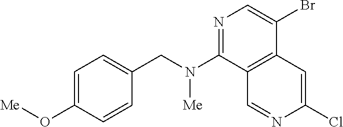 [SMILES: COc1ccc(CN(C)c2ncc(Br)c3cc(Cl)ncc23)cc1]

A solution of 6-chloro-N-(4-methoxybenzyl)-N-methyl-2,7-naphthyridin-1-amine (15.00 g; 47.770 mmol; 1.00 eq.) and NBS (8.80 g; 49.44 mmol; 1.01 eq.) in DMF (60 mL) was stirred at room temperature for 1 hour under nitrogen atmosphere. The desired product was detected via LCMS. The resulting mixture was diluted with brine (200 mL) and extracted with EtOAc (3×50 mL). The organic layers was washed with brine (2×20 mL) and dried over anhydrous Na₂SO₄. After filtration, the filtrate was concentrated under reduced pressure. The residue was purified by flash chromatography on silica gel column using 25-60% of EtOAc in petroleum ether as eluent to afford 4-bromo-6-chloro-N-(4-methoxybenzyl)-N-methyl-2,7-naphthyridin-1-amine as a brown solid (17.00 g, 90.9%). LCMS (ESI) m/z 392.0, [M+H]⁺.

### Step 3: 4-bromo-N¹,N⁶-bis(4-methoxybenzyl)-N¹-methyl-2,7-naphthyridine-1,6-diamine

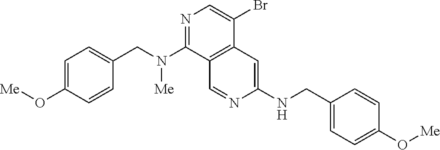 [SMILES: COc1ccc(CNc2cc3c(Br)cnc(N(C)Cc4ccc(OC)cc4)c3cn2)cc1]

To a solution of 4-bromo-6-chloro-N-(4-methoxybenzyl)-N-methyl-2,7-naphthyridin-1-amine (17.00 g; 43.29 mmol; 1.00 eq.) and (4-methoxyphenyl)methanamine (12.00 g; 87.47 mmol; 2.02 eq.) in NMP (200 mL) was added DIPEA (28.00 g; 216.63 mmol; 5.00 eq.) at room temperature. The resulting solution was stirred at 140° C. for 16 hours under nitrogen atmosphere. The desired product was detected via LCMS. The mixture was allowed to cool down to room temperature. The mixture was diluted with water (1000 mL) and extracted with CH₂Cl₂ (3×200 mL). The organic layer was washed with brine (3×100 mL) and dried over anhydrous Na₂SO₄. After filtration, the filtrate was concentrated under reduced pressure. The residue was purified by flash chromatography on silica gel column using 25-60% of EtOAc in petroleum ether as eluent to afford 4-bromo-N¹,N⁶-bis(4-methoxybenzyl)-.N¹-methyl-2,7-naphthyridine-1,6-diamine as a off-white solid (17.00 g, 79.5%). LCMS (ESI) m/z 493.1, [M+H]⁺.

### Step 4: 4-bromo-N¹-methyl-2,7-naphthyridine-1,6-diamine

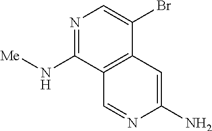 [SMILES: CNc1ncc(Br)c2cc(N)ncc12]

A solution of 4-bromo-N¹,N⁶-bis(4-methoxybenzyl)-N¹-methyl-2,7-naphthyridine-1,6-diamine (15.00 g; 30.40 mmol; 1.00 eq.) in TFA (70 mL) was stirred at 60° C. for 3 hours under nitrogen atmosphere. The desired product was detected via LCMS. The mixture was allowed to cool down to room temperature and concentrated under reduced pressure. The residue was diluted with a mixture solvent of EtOAc/water (1:1, 100 mL). The mixture was basified to pH=7 with saturated NaHCO₃ solution to precipitate solids. The precipitated solids were collected by filtration and washed with water (2×20 mL). The solids were dried under vacuum to afford 4-bromo N¹-methyl-2,7-naphthyridine-1,6-diamine as an off-white solid (6.00 g, 77.9%). LCMS (ESI) m/z 253.0, [M+H]⁺.

### Step 5: 4-bromo-5-iodo-N¹-methyl-2,7-naphthyridine-1,6-diamine

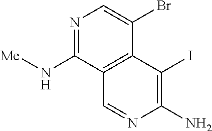 [SMILES: CNc1ncc(Br)c2c(I)c(N)ncc12]

A solution of 4-bromo-N¹-methyl-2,7-naphthyridine-1,6-diamine (2.00 g; 7.90 mmol; 1.00 eq.) and NIS (1.90 g; 8.44 mmol; 1.07 eq.) in AcOH (20 mL) was stirred at room temperature for 16 hours under nitrogen atmosphere. The desired product was detected via LCMS. The resulting mixture was concentrated under reduced pressure. The residue was dissolved in a mixture solvent of EtOAc/water (1:1, 100 mL). The mixture was basified to pH=8 with saturated NaHCO₃ solution, whereupon the precipitated product was isolated via filtration, washed with water (2×10 mL), and dried under vacuum to afford 4-bromo-5-iodo-N¹-methyl-2,7-naphthyridine-1,6-diamine as a brown solid (1.80 g, 60.1%). LCMS (ESI) m/z 378.9, [M+H]⁺.

### Step 6: 4-bromo-N¹-methyl-5-((trimethylsilyl)ethynyl)-2,7-naphthyridine-1,6-diamine

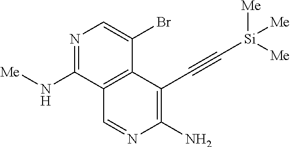 [SMILES: CNc1ncc(Br)c2c(C#C[Si](C)(C)C)c(N)ncc12]

To a solution of 4-bromo-5-iodo N¹-methyl-2,7-naphthyridine-1,6-diamine (380.0 mg; 1.00 mmol; 1.00 eq.) and ethynyltrimethylsilane (200.0 mg; 2.03 mmol; 2.03 eq.) in THF (10 mL) was added PdCl₂(PPh₃)₂ (40.0 mg; 0.05 mmol; 0.06 eq.), CuI (15.0 mg; 0.08 mmol; 0.08 eq.) and TEA (300.0 mg; 2.96 mmol; 2.96 eq.) at room temperature. The resulting mixture was stirred at room temperature for 16 hours under nitrogen atmosphere. The resulting mixture was concentrated under reduced pressure. The residue was purified by flash chromatography on silica gel column using 2-6% of methanol in dichloromethane as eluent to afford 4-bromo-N¹-methyl-5-((trimethylsilyl)ethynyl)-2,7-naphthyridine-1,6-diamine as a yellow solid (160.0 mg, 45.6%). LCMS (ESI) m/z 349.0, [M+H]⁺.

### Step 7: 4-bromo-5-ethynyl-N¹-methyl-2,7-naphthridine-1,6-diamine

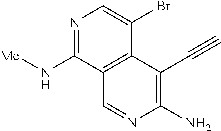 [SMILES: C#Cc1c(N)ncc2c(NC)ncc(Br)c12]

A solution of 4-bromo-N¹-methyl-5-((trimethylsilyl)ethynyl)-2,7-naphthyridine-1,6-diamine (150.0 mg; 0.42 mmol; 1.00 eq.) and K₂CO₃ (148.0 mg; 1.07 mmol; 2.49 eq.) in MeOH: (5 mL) was stirred at room temperature for 3 hours under nitrogen atmosphere. The desired product was detected via LCMS. The resulting mixture was diluted with brine (10 mL) and extracted with EtOAc (2×10 mL). The organic layers were dried over anhydrous Na₂SO₄. After filtration, the filtrate was concentrated under reduced pressure to afford 4-bromo-5-ethynyl-N¹-methyl-2,7-naphthyridine-1,6-diamine as a yellow solid (110.0 mg, 92.4%). LCMS (ESI) m/z 277.0, [M+H]⁺.

### Step 8: 9-bromo-N-methyl-3H-pyrrolo[2,3-c][2,7]naphthyridin-6-amine

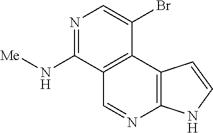 [SMILES: CNc1ncc(Br)c2c1cnc1[nH]ccc12]

A solution of 4-bromo-5-ethynyl-N¹-methyl-2,7-naphthyridine-1,6-diamine (60.0 mg; 0.21 mmol; 1.00 eq.) and t-BuOK (55.0 mg; 0.49 mmol; 2.26 eq.) in DMF (2 mL) was stirred at 80° C. for 6 hours under nitrogen atmosphere. The desired product was detected via LCMS. The 1.5 mixture was allowed to cool down to room temperature and diluted with brine (10 mL). The resulting mixture was extracted with EtOAc (2×10 mL). The organic layers were washed with brine (2×5 mL) and dried over anhydrous Na₂SO₄. After filtration, the filtrate was concentrated under reduced pressure. The residue was purified by flash chromatography on silica gel column using 2-6% of methanol in dichloromethane as eluent to afford 9-bromo-N-methyl-3H-pyrrolo[2,3-c][2,7]naphthyridin-6-amine as a brown solid (45.0 mg, 75.0%). LCMS (ESI) m/z 277.0, [M+H]⁺.

### Example 825: Synthesis of 9-(benzo[d]oxazol-2-yl)-N-methyl-3H-pyrrolo[2,3-c][2,7]naphthyridin-6-amine

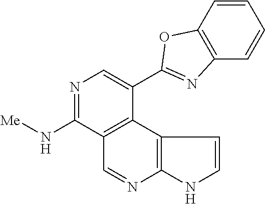 [SMILES: CNc1ncc(-c2nc3ccccc3o2)c2c1cnc1[nH]ccc12]

To a solution of 9-bromo-N-methyl-3H-pyrrolo[2,3-c][2,7]naphthyridin-6-amine (Example 824) (70.0 mg; 0.250 mmol; 1.00 eq.) and 2-(tributylstannyl)benzo[d]oxazole (620.0 mg; 1.510 mmol; 6.01 eq.) in 1,4-dioxane (5 mL) were added CuI (10.0 mg; 0.050 mmol; 0.21 eq.) and Pd(dppf)Cl₂·CH₂Cl₂ (35.0 mg; 0.040 mmol; 0.17 eq.) at room temperature. The resulting mixture was stirred at 110° C. for 7 hours under nitrogen atmosphere. The desired product was detected via LCMS. The resulting mixture was concentrated under reduced pressure. The residue was purified by flash chromatography on silica gel column using 2-6% of methanol in dichloromethane as eluent to afford 9-(benzo[d]oxazol-2-yl)-N-methyl-3H-pyrrolo[2,3-c][2,7]naphthyridin-6-amine as a light brown solid (22.8 mg, 28.6%). LCMS (ESI) m/z 316.1, [M+H]+¹11: NMR (400 MHz, DMSO-d₆) δ 12.17 (s, 1H), 9.27 (s, 1H), 8.58 (s, 1H), 8.44-8.37 (m, 1H), 7.91-7.76 (m, 2H), 7.51-7.37 (m, 3H), 6.28-6.26 (m, 1H), 3.10 (d, J=4.4 Hz, 3H).

### Example 826: Synthesis of N-(5-(3-methoxy-5-(1-methyl-1H-1,2,4-triazol-5-yl)phenethyl)-8-(methylamino)-2,7-naphthyridin-3-yl)cyclopropanecarboxamide

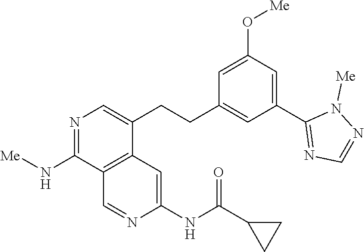 [SMILES: CNc1ncc(CCc2cc(OC)cc(-c3ncnn3C)c2)c2cc(NC(=O)C3CC3)ncc12]

### Step 1: 3-bronco-5-methoxybenzamide

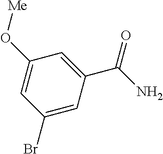 [SMILES: COc1cc(Br)cc(C(N)=O)c1]

To a solution of 3-bromo-5-methoxybenzoic acid (5.00 g; 21.641 mmol; 1.00 eq.) in CH₂Cl₂ (110 mL) was added HATU (9.90 g; 26.037 mmol; 1.20 eq.). After stirring at room temperature for 20 minutes, to the above solution were added DIPEA (8.40 g; 64.992 mmol; 3.00 eq.) and NH₄Cl (11.50 g; 214.993 mmol; 9.93 eq.). The reaction was stirred at room temperature for 1 hour. The desired product was detected via LCMS. The resulting mixture was concentrated under vacuum and purified by flash chromatography on silica gel column using 0-50% of EtOAc in CH₂Cl₂ as eluent to provide the crude product. The crude product was slurred with CH₂Cl₂ (30 mL) at room temperature for 3 hours. The precipitated solids were collected by filtration and washed with CH₂Cl₂ to afford 3-bromo-5-methoxybenzamide as a white solid (4.5 g, 91.8%). LCMS (ESI) m/z 230.0, [M+H]⁺.

### Step 2: 3-(3-bromo-5-methoxyphenyl)-1H-1,2,4-triazole

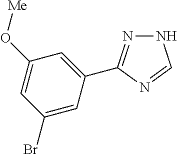 [SMILES: COc1cc(Br)cc(-c2nc[nH]n2)c1]

A solution of 3-bromo-5-methoxybenzamide (500.0 mg; 2.173 mmol; 1.00 eq.) and DMF-DMA (2.60 g; 21.833 mmol; 10.00 eq.) was stirred at 95° C. for 1 hour. The resulting mixture was concentrated under reduced pressure to get the intermediate. The intermediate was dissolved in EtOH (3 mL) and named solution A. Another solution of AcOH (4 mL) in EtOH (16 mL) was allowed to cool down to 0° C., to this solution were added hydrazine hydrate (80%) (873.4 mg; 13.957 mmol; 6.42 eq.) and solution A dropwise at −10° C. The reaction was stirred at room temperature for 4 hours. The desired product was detected via LCMS. The EtOH was removed under reduced pressure, the remaining mixture was added to water (50 mL) to precipitate solids. The precipitated solids were collected by filtration and washed with water (3×10 mL) and dried under reduced pressure to provide 3-(3-bromo-5-methoxyphenyl)-1H-1,2,4-triazole as a yellow solid (1.00 g, crude). LCMS (ESI) m/z 254.0, [M+H]⁺.

### Step 3: 3-(3-bromo-5-methoxyphenyl)-.1-methyl-1H-1,2,4-triazole and 5-(3-bromo-5-methoxyphenyl)-1-methyl-1H-1,2,4-triazole

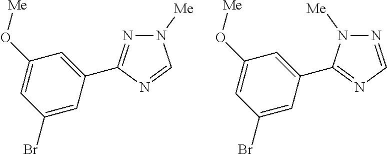 [SMILES: COc1cc(Br)cc(-c2ncn(C)n2)c1 ; COc1cc(Br)cc(-c2ncnn2C)c1]

To a solution of 3-(3-bromo-5-methoxyphenyl)-1H-1,2,4-triazole (1.00 g; 3.936 mmol; 1.00 eq.) in DMF (7 mL) was added K₂CO₃ (1.10 g; 7.959 mmol; 2.02 eq.) and MeI (841.3 mg; 5.927 mmol; 1.51 eq.). The reaction was stirred at room temperature overnight. The desired product was detected via LCMS. The resulting mixture was diluted with EtOAc (100 mL) and washed with brine (3×20 mL). The organic layer was dried over anhydrous Na₂SO₄, filtered, and concentrated under vacuum. The residue was purified by flash chromatography on silica gel column using 10-50% of EtOAc in petroleum ether as eluent to provide 3-(3-bromo-5-methoxyphenyl)-1-methyl-1H-1,2,4-triazole as a white solid (160 mg, 15%) and 5-(3-bromo-5-methoxyphenyl)-1-methyl-1H-1,2,4-triazole as a white solid (60.0 mg, 5.7%). LCMS (ESI) m/z 268.0, [M+H]⁺.

### Step 4: 5-(3-methoxy-5-vinylphenyl)-1-methyl-1H-1,2,4-triazole

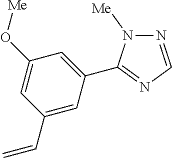 [SMILES: C=Cc1cc(OC)cc(-c2ncnn2C)c1]

A solution of S-Phos (39.4 mg; 0.096 mmol; 0.10 eq.), K₃PO₄ (595.5 mg; 2.805 mmol; 3.01 eq.), Pd(OAc)₂ (10.7 mg; 0.048 mmol; 0.05 eq.),5-(3-bromo-5-methoxyphenyl)-1-methyl-1H-1,2,4-triazole (250.0 mg; 0.932 mmol; 1.00 eq.) in a mixture solvent of 1,4-dioxane/water (5:1, 3 mL) was added 4,4,5,5-tetramethyl-2-vinyl-1,3,2-dioxaborolane (158.6 mg; 1.030 mmol; 1.10 eq.). The reaction was stirred at 80° C. overnight. The desired product was detected via LCMS. The mixture was concentrated under reduced pressure. The residue was purified by flash chromatography on silica gel column using 0-60% of EtOAc in petroleum ether as eluent to provide 5-(3-methoxy-5-vinylphenyl)-1-methyl-1H-1,2,4-triazole as a yellow oil (180.0 mg, 89.1%). LCMS (ESI) m/z 216.1, [M+H]⁺.

### Step 5: (E)-N-(5-(3-methoxy-5-(1-methyl-1H-1,2,4-triazol-5-yl)styryl)-8-(methylamino)-2,7-naphthyridin-3-yl)cyclopropanecarboxamide

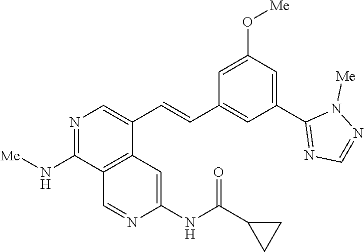 [SMILES: CNc1ncc(/C=C/c2cc(OC)cc(-c3ncnn3C)c2)c2cc(NC(=O)C3CC3)ncc12]

To a solution of Pd(PPh₃)₂Cl₂ (21.9 mg; 0.031 mmol; 0.10 eq.), Na₂CO₃ (66.3 mg; 0.626 mmol; 2.01 eq.), N-(5-bromo-8-(methylamino)-2,7-naphthyridin-3-yl)cyclopropanecarboxamide (100.0 mg; 0.311 mmol; 1.00 eq.) in DMF (3 mL) was added 5-(3-methoxy-5-vinylphenyl)-1-methyl-1H-1,2,4-triazole (100.8 mg; 0.468 mmol; 1.50 eq.). The reaction was stirred at 100° C. overnight under nitrogen atmosphere. The resulting mixture was diluted with EtOAc (80 mL), washed with brine (3×20 mL). The organic layer was dried over anhydrous Na₂SO₄, filtered and the filtrate was concentrated under reduced pressure. The residue was purified by flash chromatography on silica gel column using 2-10% of MeOH in CH₂Cl₂ as eluent to provide the crude product. The product was purified by flash chromatography on pre-packed C18 column using 0-100% MeCN in water (10 mmol/L NH₄HCO₃) as eluent to provide (E)-N-(5-(3-methoxy-5-(1-methyl-1H-1,2,4-triazol-5-yl)styryl)-8-(methylamino)-2,7-naphthyridin-3-yl)cyclopropanecarboxamide as a yellow solid (52 mg, 36%). LCMS (ESI) m/z 456.2, [M+H]⁺.

### Step 6: N-(5-(3-methoxy-5-(1-methyl-1H-1,2,4-triazol-5-yl)phenethyl)-8-(methylamino)-2,7-naphthyridin-3-yl)cydopropanecarboxamide

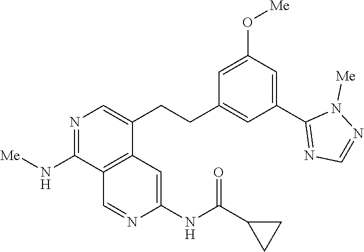 [SMILES: CNc1ncc(CCc2cc(OC)cc(-c3ncnn3C)c2)c2cc(NC(=O)C3CC3)ncc12]

To a solution of (E)-N-(5-(3-methoxy-5-(1-methyl-1H-1,2,4-triazol-5-yl)styryl)-8-(methylamino)-2,7-naphthyridin-3-yl)cyclopropanecarboxamide (52.0 mg; 0.114 mmol; 1.00 eq.) in MeOH (250 mL) was added 10% Pd/C (52.0 mg; 100% w/w) under nitrogen atmosphere. The mixture was hydrogenated at room temperature overnight under hydrogen atmosphere (2 atm). After filtration, the filtrate was concentrated under reduced pressure. The residue was purified by reverse phase preparative HPLC (Prep-C18, 5 μM XBridge OBD column, 30×150 mm, waters; gradient elution of 30-40% MeCN in water over a 10 min period, where both water and MeCN contain 10 mmol/L NH₄HCO₃, flow rate: 60 mL/min, detector UV wavelength: 254 nm) to afford the the crude product. The product was purified by Prep-Achiral-SFC (DAICEL DCpak P4VP, 3×25 cm, 5 μm; Mobile Phase A: CO₂, Mobile Phase B: MeOH (0.1% 2 M NH₃-MeOH); Flow rate: 60 mL/min; Gradient: isocratic 30% B; Wave Length: 220/254 nm) to afford N-(5-(3-methoxy-5-(1-methyl-1H-1,2,4-triazol-5-yl)phenethyl)-8-(methylamino)-2,7-naphthyridin-3-yl)cyclopropanecarboxamide as a white solid (7.4 mg, 14.0%). LCMS (ESI) m/z 485.2, [M+H]⁺.

### Examples 827 and 828: Synthesis of N-(5-(4-((1-methyl-1H-1,2,4-triazol-3-yl)methyl)oxazol-2-yl)-8-(methylamino)-2,7-naphthyridin-3-yl)cyclopropanecarboxamide (Example 827) and N-(5-(4-((1-methyl-1H-1,2,4-triazol-5-yl)methyl)oxazol-2-yl)-8-(methylamino)-2,7-naphthyridin-3-yl)cyclopropanecarboxamide (Example 828)

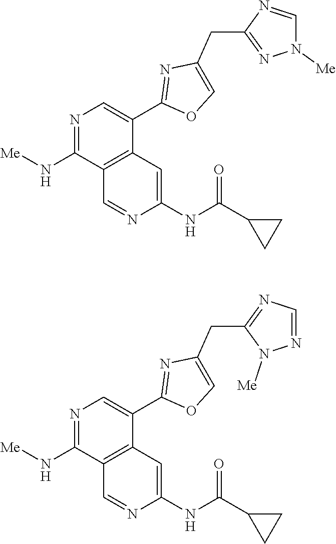 [SMILES: CNc1ncc(-c2nc(Cc3ncn(C)n3)co2)c2cc(NC(=O)C3CC3)ncc12 ; CNc1ncc(-c2nc(Cc3ncnn3C)co2)c2cc(NC(=O)C3CC3)ncc12]

### Step 1: (2-chlorooxazol-4-yl)methanol

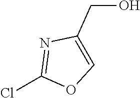

To a solution of ethyl 2-chlorooxazole-4-carboxylate (2.00 g; 11.390 mmol; 1.00 eq.) in CH₂Cl₂ (30 mL) was added diisobutylaluminum hydride (1.0 M in CH₂Cl₂, 17.1 mL) at −78° C. under nitrogen atmosphere. The resulting mixture was stirred at room temperature for 1.6 hours under nitrogen atmosphere. The reaction was quenched with ice water. The pH value of the mixture was adjusted to 2 with 1 M HCl aqueous solution. The resulting mixture was extracted with CH₂Cl₂ (3×100 mL). The combined organic layers were washed with brine (3×30 mL), dried over anhydrous sodium sulfate, and filtered. The filtrate was concentrated under reduced pressure and purified by flash chromatography on silica gel column using 10-40% of EtOAc in petroleum ether as eluent to provide (2-chlorooxazol-4-yl)methanol as a yellow oil (1.2 g, 79.0%). LCMS (ESI) m/z 134.0, [M+H]⁺.

### Step 2: (2-chlorooxazol-4-yl)methyl methanesulfonate

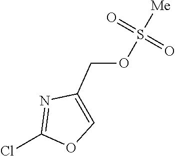 [SMILES: CS(=O)(=O)OCc1coc(Cl)n1]

To a solution of (2-chlorooxazol-4-yl)methanol (1.20 g; 8.990 mmol; 1.00 eq.) and triethylamine (1.81 g; 17.980 mmol; 2.00 eq.) in CH₂Cl₂ (20 mL) was added a solution of methanesulfonic anhydride (1.70 g; 9.890 mmol; 1.1.0 eq.) in CH₂Cl₂ (20 mL) at 0° C. The mixture was stirred at room temperature for 16 hours under nitrogen atmosphere. The reaction was quenched with water (50 mL). The resulting mixture was extracted with CH₂Cl₂ (3×30 mL). The combined organic layers were dried over anhydrous sodium sulfate and filtered. The filtrate was concentrated under reduced pressure to afford (2-chlorooxazol-4-yl)methyl methanesulfonate as a yellow oil (1.7 g, 89.0%). LCMS (ESI) m/z 212.0, [M+H]⁺.

### Step 3: 2-(2-chlorooxazol-4-yl)acetonitrile

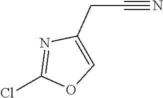

To a solution of (2-chlorooxazol-4-yl)methyl methanesulfonate (1.70 g; 8.030 mmol; 1.00 eq.) in acetonitrile (20 mL) was added tetrabutylammonium fluoride (1 M in THF, 16.1 mL) and trimethylsilanecarbonitrile (1.60 g; 16.070 mmol; 2.00 eq.) at room temperature. The resulting mixture was stirred at room temperature for 16 hours under nitrogen atmosphere. The reaction was quenched with water (20 mL). The resulting mixture was extracted with EtOAc (3×30 mL). The combined organic layers were dried over anhydrous sodium sulfate and filtered. The filtrate was concentrated under vacuum. The residue was purified by flash chromatography on silica gel column using 0-10% of MeOH in CH₂Cl₂ as eluent to provide 2-(2-chlorooxazol-4-yl)acetonitrile as a white solid (664.0 mg, 58.0%). LCMS (ESI) m/z 143.0, [M+H]⁺.

### Step 4: N-(5-(4-(cyanomethyl)oxazol-2-yl)-8-(methylamino)-2,7-naphthyridin-3-yl)cyclopropanecarboxamide

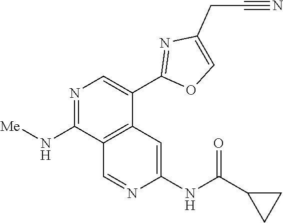 [SMILES: CNc1ncc(-c2nc(CC#N)co2)c2cc(NC(=O)C3CC3)ncc12]

A solution of 2-(2-chlorooxazol-4-yl)acetonitrile (50.0 mg; 0.350 mmol; 1.00 eq.), N-(8-(methylamino)-5-(4,4,5,5-tetramethyl-1,3,2-dioxaborolan-2-yl)-2,7-naphthyridin-3-yl)cyclopropanecarboxamide (129.2 mg; 0.35 mmol; 1.00 eq.), XPhos Pd G₃ (29.7 mg; 0.04 mmol; 0.10 eq.), XPhos (16.7 mg; 0.04 mmol; 0.10 eq.) and K₃PO₄. (223.4 mg; 1.05 mmol; 3.00 eq.) in a mixture solvent of 1,4-dioxane/water (5:1, 3 mL) was stirred at 90° C. for 2 hours under nitrogen atmosphere. The desired product was detected via LCMS. The mixture was concentrated under vacuum. The residue was purified by flash chromatography on silica gel column using 0-25% of MeOH in CH₂Cl₂ as eluent to provide N-(5-(4-(cyanomethyl)oxazol-2-yl)-8-(methylamino)-2,7-naphthyridin-3-yl)cyclopropanecarboxamide as a light yellow solid (100.0 mg, 81.9%). LCMS (ESI) in/z 349.1, [M+H]⁺.

### Step 5: N-(5-(4-((1H-1,2,4-triazol-3-yl)methyl)oxazol-2-yl)-8-(methylamino)-2,7-naphthyridin-3-yl)cyclopropanecarboxamide

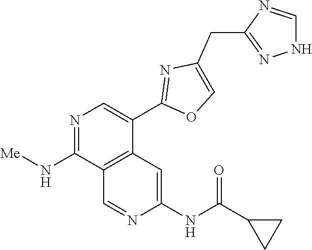 [SMILES: CNc1ncc(-c2nc(Cc3nc[nH]n3)co2)c2cc(NC(=O)C3CC3)ncc12]

To a solution of N-(5-(4-(cyanomethyl)oxazol-2-yl)-8-(methylamino)-2,7-naphthyridin-3-yl)cyclopropanecarboxamide (163.0 mg; 0.47 mmol; 1.00 eq.) in MeOH (10 mL) was added N-formylhydrazine (70.2 mg; 1.17 mmol; 2.50 eq.) and K₂CO₃ (84.1 mg; 0.61 mmol; 1.30 eq.). The resulting mixture was stirred at 60° C. for 16 hours. The desired product was detected via LCMS. After the reaction was completed, the mixture was cooled to room temperature and concentrated under vacuum. The residue was purified by flash chromatography on silica gel column using 0-25% of MeOH in CH₂Cl₂ as eluent to provide N-(5-(4-((1H-1,2,4-triazol-3-yl)methyl)oxazol-2-yl)-8-(methylamino)-2,7-naphthyridin-3-yl)cyclopropanecarboxamide as a yellow solid (106.0 mg, 58.1%). LCMS (ESI) m/z 391.2, [M+H]⁺.

### Step 6: N-(5-(4-((1-methyl-1H-1,2,4-triazol-3-yl)methyl)oxazol-2-yl)-8-(methylamino)-2,7-naphthyridin-3-yl)cyclopropanecarboxamide and N-(5-(4-((1-methyl-1H-1,2,4-triazol-5-yl)methyl)oxazol-2-yl)-8-(methylamino)-2,7-naphthyridin-3-yl)cyclopropanecarboxamide

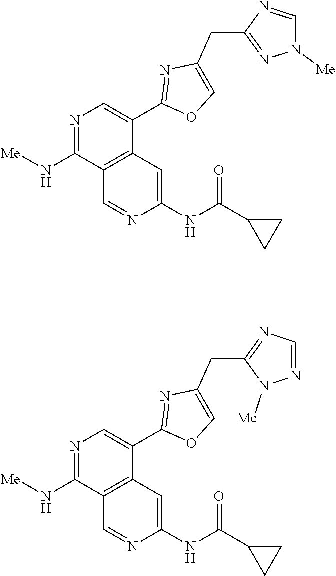 [SMILES: CNc1ncc(-c2nc(Cc3ncn(C)n3)co2)c2cc(NC(=O)C3CC3)ncc12 ; CNc1ncc(-c2nc(Cc3ncnn3C)co2)c2cc(NC(=O)C3CC3)ncc12]

To a solution of N-(5-(4-((1.π-1,2,4-triazol-3-yl)methyl)oxazol-2-yl)-8-(methylamino)-2,7-naphthyridin-3-yl)cyclopropanecarboxamide (96.0 mg; 0.250 mmol; 1.00 eq.) in DMF (2 mL) were added iodomethane (34.9 mg; 0.250 mmol; 1.00 eq.) and K₂CO₃ (51.0 mg; 0.370 mmol; 1.50 eq.). The resulting mixture was stirred at room temperature for 16 hours. The desired product was detected via LCMS. After the reaction was completed, the mixture was diluted with water (10 mL). The resulting mixture was extracted with EtOAc (3×30 mL). The combined organic layers were washed with brine (2×10 mL), dried over anhydrous sodium sulfate and filtered. The filtrate was concentrated under vacuum and purified by flash chromatography on silica gel column using 0-25% of MeOH in CH₂Cl₂ as eluent to provide a mixture of two products. The mixture product was repurified by reverse phase preparative HPLC (Prep-C18, 5 μM XBridge OBD column, 30×150 mm, waters; gradient elution of 20-30% MeCN in water over a 10 min period, where water contain 10 mmol/L NH₄HCO₃, flow rate: 60 mL/min, detector UV wavelength: 254 nm) to provide N-(5-(4-((1-methyl-1H-1,2,4-triazol-3-yl)methyl)oxazol-2-yl)-8-(methylamino)-2,7-naphthyridin-3-yl)cyclopropanecarboxamide (Example 827) as a light yellow solid (12.0 mg, 12.0%) and N-(5-(4-((1-methyl-1H-1,2,4-triazol-5-yl)methyl)oxazol-2-yl)-8-(methylamino)-2,7-naphthyridin-3-yl)cyclopropanecarboxamide (Example 828) as an off-white solid (9.6 mg, 10.0%). LCMS (ESI) m/z 405.2, [M+H]⁺.

### Example 829: Synthesis of N-(5-(4-(1H-1,2,4-triazol-3-yl)oxazol-2-yl)-8-(methylamino)-2,7-naphthyridin-3-yl)cyclopropanecarboxamide

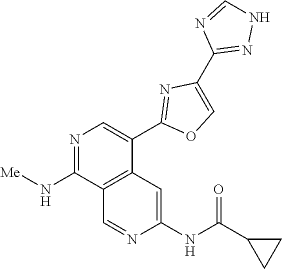 [SMILES: CNc1ncc(-c2nc(-c3nc[nH]n3)co2)c2cc(NC(=O)C3CC3)ncc12]

### Step 1: N-(5-(4-cyanooxazol-2-yl)-8-(methylamino)-2,7-naphthyridin-3-yl)cyclopropanecarboxamide

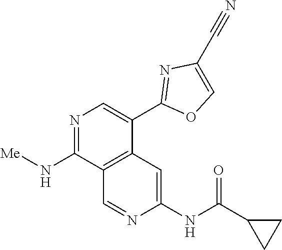 [SMILES: CNc1ncc(-c2nc(C#N)co2)c2cc(NC(=O)C3CC3)ncc12]

A solution of N-(8-(methylamino)-5-(4,4,5,5-tetramethyl-1,3,2-dioxaborolan-2-yl)-2,7-naphthyridin-3-yl)cyclopropanecarboxamide (150.0 mg; 0.407 mmol; 1.00 eq.), 2-chlorooxazole-4-carbonitrile (52.5 mg; 0.408 mmol; 1.00 eq.), XPhos Pd G₃ (34.5 mg; 0.041 mmol; 0.10 eq.), XPhos (19.4 mg; 0.041 mmol; 0.10 eq.) and K₃PO₄ (259.0 mg; 1.220 mmol; 3.00 eq.) in a mixture solvent of 1,4-dioxane/water (5:1, 3 mL) was stirred at 90° C. for 1 hour under nitrogen atmosphere. The resulting mixture was concentrated under reduced pressure. The residue was purified by flash chromatography on silica gel column using 0-25% of MeOH in CH₂Cl₂ as eluent to provide N-(5-(4-cyanooxazol-2-yl)-8-(methylamino)-2,7-naphthyridin-3-yl)cyclopropanecarboxamide as a light yellow solid (90.0 mg, 66.0%). LCMS (ESI) m/z 335.1, [M+H]⁺.

### Step 2: N-(5-(4-(1H-1,2,4-triazol-3-yl)oxazol-2-yl)-ethylamino)-2,7-naphthyridin-3-yl)cyclopropanecarboxamide

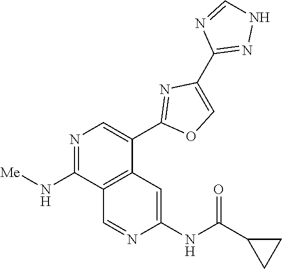 [SMILES: CNc1ncc(-c2nc(-c3nc[nH]n3)co2)c2cc(NC(=O)C3CC3)ncc12]

To a solution of N-(5-(4-cyanooxazol-2-yl)-8-(methylamino)-2,7-naphthyridin-3-yl)cyclopropanecarboxamide (90.0 mg; 0.269 mmol; 1.00 eq.) and K₂CO₃ (45.7 mg; 0.331 mmol; 1.23 eq.) in MeOH (6 mL) was added formohydrazide (48.4 mg; 0.807 mmol; 3.00 eq.). The reaction was stirred at 70° C. overnight under nitrogen atmosphere. The resulting mixture was concentrated under reduced pressure. The residue was purified by flash chromatography on silica gel column using 0-25% of MeOH in CH₂Cl₂ as eluent to provide 80 mg of the desired product. 10 mg of the product was purified by flash chromatography on pre-packed C18 column using 0-40% of MeCN in water (10 mmol/L NH₄HCO₃) as eluent to provide N-(5-(4-(1H-1,2,4-triazol-3-yl)oxazol-2-yl)-8-(methylamino)-2,7-naphthyridin-3-yl)cyclopropanecarboxamide as an off-white solid (4.8 mg, 3.88%). LCMS (ESI) m/z 377.1, [M+H]⁺. ¹H NMR (400 MHz, DMSO-d₆) δ 14.20 (s, 1H), 11.03 (s, 1H), 9.44 (s, 1H), 9.23 (s, 1H), 8.70 (s, 1H), 8.68 (s, 1H), 8.55-8.48 (m, 1H), 8.45 (s, 1H), 3.07 (d, J=4.4 Hz, 3H), 2.13-2.03 (m, 1H), 0.91-0.78 (m, 4H).

### Examples 830 and 831: Synthesis of N-(5-(4-(1-methyl-1H-1,2,4-triazol-3-yl)oxazol-2-yl)-8-(methylamino)-2,7-naphthyridin-3-yl)cyclopropanecarboxamide (Example 830) and N-(5-(4-(1-methyl-1H-1,2,4-triazol-5-yl)oxazol-2-yl)-8-(methylamino)-2,7-naphthyridin-3-yl)cyclopropanecarboxamide (Example 831)

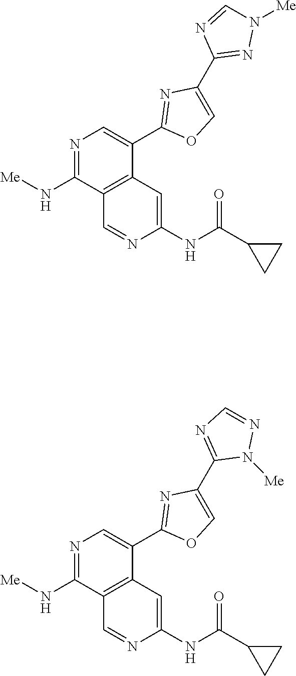 [SMILES: CNc1ncc(-c2nc(-c3ncn(C)n3)co2)c2cc(NC(=O)C3CC3)ncc12 ; CNc1ncc(-c2nc(-c3ncnn3C)co2)c2cc(NC(=O)C3CC3)ncc12]

To a solution of N-(5-(4-(1H-1,2,4-triazol-3-yl)oxazol-2-yl)-8-(methylamino)-2,7-naphthyridin-3-yl)cyclopropanecarboxamide (Example 829) (90.0 mg; 0.239 mmol; 1.00 eq.) and K₂CO₃ (49.5 mg; 0.358 mmol; 1.50 eq.) in DMF (3 mL) was added MeI (30.6 mg; 0.216 mmol; 0.90 eq.). The reaction was stirred at room temperature for 14 hours. The desired product was detected via LCMS. The resulting mixture was purified by flash chromatography on pre-packed C18 column using 20-80% of MeOH in water (10 mmol/L NH₄HCO₃) to provide the crude product. The crude product was purified by reverse phase preparative HPLC (Prep-C18, 5 μM Xselect CSH F-Phenyl OBD column, 19×250 mm, waters; gradient elution of 35-40% MeCN in water over a 10 min period, where water contain 0.1% formic acid, flow rate: 20 mL/min, detector UV wavelength: 254 nm) to afford N-(5-(4-(1-methyl-1H-1,2,4-triazol-3-yl)oxazol-2-yl)-8-(methylamino)-2,7-naphthyridin-3-yl)cyclopropanecarboxamide (Example 830) as a light yellow solid (5.1 mg, 5%) and N-(5-(4-(1-methyl-1H-1,2,4-triazol-5-yl)oxazol-2-yl)-8-(methylamino)-2,7-naphthyridin-3-yl)cyclopropanecarboxamide (Example 831) as a light yellow solid (9.7 mg, 10.4%). LCMS (ESI) m/z 391.2, [M+H]⁺.

HNMR for Example 830: ¹H NMR (400 MHz, DMSO-d₆) δ 11.00 (s, 1H), 9.42 (s, 1H), 9.19 (s, 1H), 8.69 (s, 1H), 8.61 (s, 1H), 8.55 (s, 1H), 8.49-8.48 (m, 1H), 3.94 (s, 3H), 3.06 (d, J=4.4 Hz, 3H), 2.1.1-2.03 (m, 1H), 0.91-0.79 (m, 4H).

HNMR for Example 831: LCMS (ESI) m/z 391.2, [M+H]⁺. ¹H NMR (400 MHz, DMSO-d₆) δ 11.07 (s, 1H), 9.68 (s, 1H), 9.43 (s, 1H), 8.79 (s, 1H), 8.78 (s, 1H), 8.59-8.58 (m, 1H), 8.02 (s, 1H), 4.34 (s, 3H), 3.07 (d, J=4.4 Hz, 3H), 2.11-2.05 (m, 1H), 0.90-0.84 (m, 4H).

### Example 832: Synthesis of N-(5-(5-(1H-1,2,4-triazol-3-yl)cyclohex-1-en-1-yl)-8-(methylamino)-2,7-naphthyridin-3-yl)cyclopropanecarboxamide

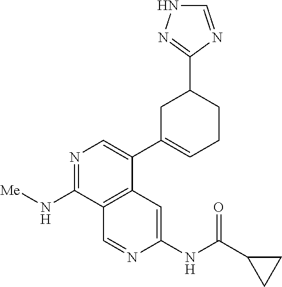 [SMILES: CNc1ncc(C2=CCCC(c3nc[nH]n3)C2)c2cc(NC(=O)C3CC3)ncc12]

### Step 1: ethyl 3-(((trifluoromethyl)sulfonyl)oxy)cyclohex-3-ene-1-carboxylate

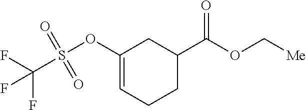 [SMILES: CCOC(=O)C1CCC=C(OS(=O)(=O)C(F)(F)F)C1]

To a solution of ethyl 3-oxocyclohexane-1-carboxylate (1.0 g; 5.875 mmol; 1.00 eq.) and 2,6-di-tert-butylpyridine (1.3 g; 6.795 mmol; 1.16 eq.) in dichloroethane (20 mL) was added trifluoromethanesulfonic anhydride (1.7 g; 6.026 mmol; 1.03 eq.) dropwise at 0° C. under nitrogen atmosphere. The resulting solution was stirred at 0° C. for 0.5 hour and then at room temperature for additional 12 hours. The reaction mixture was concentrated under vacuum. The residue was diluted with EtOAc (100 mL) and washed by brine (2×30 mL). The organic layer was dried over anhydrous sodium sulfate and concentrated under vacuum. The residue was purified by flash chromatography on silica gel column using 15-20% of EtOAc in petroleum ether as eluent to provide ethyl 3-(((trifluoromethyl)sulfonyl)oxy)cyclohex-3-ene-1-carboxylate as a colorless oil (1.40 g, 79.1%).

### Step 2: ethyl 3-(6-(cyclopropanecarboxamido)-1-(methylamino)-2,7-naphthyridin-4-yl)cyclohex-3-ene-1-carboxylate

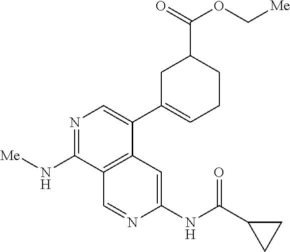 [SMILES: CCOC(=O)C1CCC=C(c2cnc(NC)c3cnc(NC(=O)C4CC4)cc23)C1]

To a solution of N-(8-(methylamino)-5-(4,4,5,5-tetramethyl-1,3,2-dioxaborolan-2-yl)-2,7-naphthyridin-3-yl)cyclopropanecarboxamide (500.0 mg; 0.679 mmol; 1.00 eq., 50%) and ethyl 3-(((trifluoromethyl)sulfonyl)oxy)cyclohex-3-ene-1-carboxylate (205.0 mg; 0.678 mmol; 1.00 eq.) in a mixture solvent of 1,4-dioxane/water (5:1, 12 mL) were added XPhos Pd CT3 (115.0 mg; 0.136 mmol; 0.20 eq.), XPhos (65.0 mg; 0.136 mmol; 0.20 eq.) and K₃PO₄ (288.0 mg; 1.357 mmol; 2.00 eq.) under nitrogen atmosphere. The reaction was stirred at 90° C. for 2 hours. The reaction was concentrated under vacuum. The residue was purified by flash chromatography on silica gel column using 10-20% of methanol in dichloromethane as eluent to provide ethyl 3-(6-(cyclopropanecarboxamido)-1-(methylamino)-2,7-naphthyridin-4-yl)cyclohex-3-ene-1-carboxylate as a off-white solid (150 mg, 56.1%). LCMS (ESI) m/z 395.2 [M+H]⁺.

### Step 3: 3-(6-(cyclopropanecarboxamido)-1-(methylamino)-2,7-naphthyridin-4-yl)cyclohex-3-ene-1-carboxylic acid

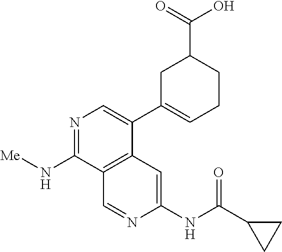 [SMILES: CNc1ncc(C2=CCCC(C(=O)O)C2)c2cc(NC(=O)C3CC3)ncc12]

To a solution of ethyl 3-(6-(cyclopropanecarboxamido)-1-(methylamino)-2,7-naphthyridin-4-yl)cyclohex-3-ene-1-carboxylate (150.0 mg; 0.380 mmol; 1 eq.) in THF (6 mL) was added a solution of LiOH·H₂O (90.0 mg; 2.145 mmol; 5.64 eq.) in water (2 mL). The resulting solution was stirred at room temperature for 12 hours. The mixture was adjusted to pH=3 with 2 M HCl and extracted with EtOAc (2×20 mL). The organic layers were combined and dried over anhydrous sodium sulfate. After filtration, the filtrate was concentrated under reduced pressure to provide 3-(6-(cyclopropanecarboxamido)-1-(methylamino)-2,7-naphthyridin-4-yl)cyclohex-3-ene-1-carboxylic acid as a white solid (100 mg, 71.7%). LCMS (ESI) m/z 367.2 [M+H]⁺.

### Step 4: 3-(6-(cyclopropanecarboxamido)-1-(methylamino)-2,7-naphthyridin-4-yl)cyclohex-3-ene-1-carboxamide

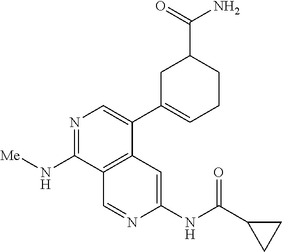 [SMILES: CNc1ncc(C2=CCCC(C(N)=O)C2)c2cc(NC(=O)C3CC3)ncc12]

To a solution of 3-(6-(cyclopropanecarboxamido)-1-(methylamino)-2,7-naphthyridin-4-yl)cyclohex-3-ene-1-carboxylic acid (110.0 mg; 0.300 mmol; 1.00 eq.) in DMF (5 mL) were added HATU (140.0 mg; 0.368 mmol; 1.23 eq.), NH₄Cl (165.0 mg; 3.085 mmol; 10.28 eq.) and DIEA (130.0 mg; 1.006 mmol; 3.35 eq.) under nitrogen atmosphere. The resulting solution was stirred at room temperature for 2 hours. The desired product was detected via LCMS. The reaction mixture was purified by flash chromatography on pre-packed C18 column using 20-70% of MeCN in water (10 mmol/L NH₄HCO₃) as eluent to provide 3-(6-(cyclopropanecarboxamido)-1-(methylamino)-2,7-naphthyridin-4-yl)cyclohex-3-ene-1-carboxamide as a white solid (75 mg, 68.3%). LCMS (ESI) m/z 366.2 [M+H]⁺.

### Step 5: N-(5-(5-(1H-1,2,4-triazol-3-yl)cyclohex-1-en-1-yl)-8-(methylamino)-2,7-naphthyridin-3-yl)cyclopropanecarboxamide

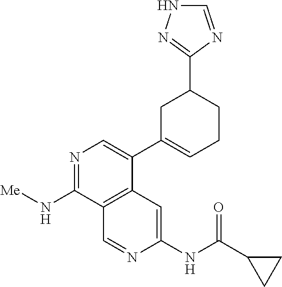 [SMILES: CNc1ncc(C2=CCCC(c3nc[nH]n3)C2)c2cc(NC(=O)C3CC3)ncc12]

A solution of 3-(6-(cyclopropanecarboxamido)-1-(methylamino)-2,7-naphthyridin-4-yl)cyclohex-3-ene-1-carboxamide (70.0 mg; 0.192 mmol; 1.00 eq.) in DMF-DMA (250 mg; 2.098 mmol; 10.95 eq.) was stirred at 95° C. for 0.5 hour under nitrogen atmosphere. The reaction was concentrated under vacuum to afford the intermediate. The intermediate was redissolved in EtOH (2 mL) and added dropwise to a solution of hydrazine hydrate (105.0 mg; 2.097 mmol; 10.95 eq.) in a mixture solvent of EtOH/AcOH (5:1, 18 mL) at −10° C. The resulting mixture was stirred at room temperature for 12 hours. The desired product was detected via LCMS. The solvent was concentrated under vacuum and purified by flash chromatography on pre-packed C18 column using 50-70% of MeCN in water (10 mmol/L NH₄HCO₃) as eluent to provide N-(5-(5-(1H-1,2,4-triazol-3-yl)cyclohex-1-en-1-yl)-8-(methylamino)-2,7-naphthyridin-3-yl)cyclopropanecarboxamide as a white solid (70 mg, 93.8%). LCMS (ESI) m/z 390.2 [M+H]⁺.

### Example 833: Synthesis of N-(5-(4-(1H-1,2,4-triazol-3-yl)cyclopent-1-en-1-yl)-8-(methylamino)-2,7-naphthyridin-3-yl)cyclopropanecarboxamide

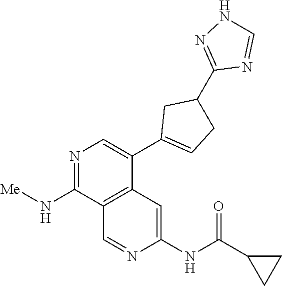 [SMILES: CNc1ncc(C2=CCC(c3nc[nH]n3)C2)c2cc(NC(=O)C3CC3)ncc12]

### Step 1: ethyl 3-(((trifluoromethyl)sulfonyl)oxy)cyclopent-3-ene-1-carboxylate

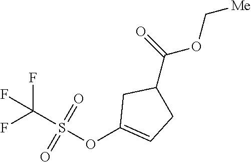 [SMILES: CCOC(=O)C1CC=C(OS(=O)(=O)C(F)(F)F)C1]

A solution of ethyl 3-oxocyclopentane-1-carboxylate (1.00 g; 6.400 mmol; 1.00 eq.) and 2,6-di-tert-butylpyridine (1.35 g; 7.040 mmol; 1.1.0 eq.) in dichloroethane (1.0 mL) was stirred at 0° C. for 30 minutes under nitrogen atmosphere. To the above solution was added triflic anhydride (1.99 g; 7.040 mmol; 1.10 eq.) dropwise at 0° C. The mixture was stirred at room temperature for 4 hours under nitrogen atmosphere. The desired product was detected via LCMS. The reaction was quenched by the addition of water (5 mL) at room temperature. The resulting mixture was diluted with CH₂Cl₂ (100 mL), washed with brine (2×10 mL), dried over anhydrous Na₂SO₄. After filtration, the filtrate was concentrated under reduced pressure. The residue was purified by flash chromatography on silica gel column using 7-20% of EtOAc in petroleum ether as eluent to provide ethyl 3-(((trifluoromethyl)sulfonyl)oxy)cyclopent-3-ene-1-carboxylate as a colorless solid (1.13 g, 61.2%). LCMS (ESI) m/z 289.0, [M+H]⁺.

### Step 2: ethyl 3-(6-(cyclopropanecarboxamido)-1-(methylamino)-2,7-naphthyridin-4-yl)cyclopent-3-ene-1-carboxylate

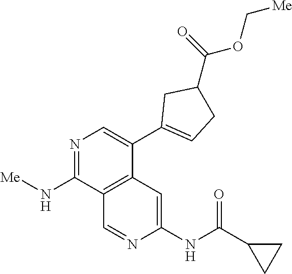 [SMILES: CCOC(=O)C1CC=C(c2cnc(NC)c3cnc(NC(=O)C4CC4)cc23)C1]

A solution of ethyl 3-(trifluoromethanesulfonyloxy)cyclopent-3-ene-1-carboxylate (300 mg; 1.04 mmol; 1.00 eq.), XPhos Pd G₃ (88.1 mg; 0.104 mmol; 0.10 eq.), XPhos (99.2 mg; 0.208 mmol; 0.20 eq.), K₃PO₄ (441 mg; 2.08 mmol; 2.00 eq.) and N-(8-(methylamino)-5-(4,4,5,5-tetramethyl-1,3,2-dioxaborolan-2-yl)-2,7-naphthyridin-3-yl)cyclopropanecarboxamide (383 mg; 1.041 mmol; 1.00 eq.) in a mixture solvent of 1,4-dioxane/water (5:1, 6 mL) was stirred at 90° C. for 1 hour under nitrogen atmosphere. The resulting mixture was concentrated under reduced pressure. The residue was purified by flash chromatography on silica gel column using 3-7% of MeOH in CH₂Cl₂ as eluent to provide ethyl 3-(6-(cyclopropanecarboxamido)-1-(methylamino)-2,7-naphthyridin-4-yl)cyclopent-3-ene-1-carboxylate as an off-white solid (270.0 mg, 70.8%). LCMS (ESI) m/z 381.0, [M+H]⁺.

### Step 3: 3-(6-(cyclopropanecarboxamido-1-(methylamino)-2,7-naphthyridin-4-yl)cyclopent-3-ene-1-carboxylic acid

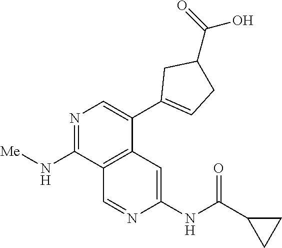 [SMILES: CNc1ncc(C2=CCC(C(=O)O)C2)c2cc(NC(=O)C3CC3)ncc12]

To a solution of 3-(6-(cyclopropanecarboxamido)-1-(methylamino)-2,7-naphthyridin-4-yl)cyclopent-3-ene-1-carboxylate (270.0 mg; 0.710 mmol; 1.00 eq.) in THF (3 mL) was added a solution of LiOH·H₂O (33.9 mg; 1.42 mmol; 2.00 eq.) in water. (1 mL). The mixture was stirred for 14 hours at room temperature. The THF was removed under reduced pressure. The resulting mixture was acidified to pH=5 with 2 M HCl solution. The mixture was purified by flash chromatography on pre-packed C18 column using 20-100% of MeCN in water (0.05% formic acid) as eluent to provide 3-(6-(cyclopropanecarboxamido)-1-(methylamino)-2,7-naphthyridin-4-yl)cyclopent-3-ene-1-carboxylic acid as an off-white solid (220.0 mg, 87.9%). LCMS (ESI) m/z 353.2, [M+H]⁺.

### Step 4: 3-(6-(cyclopropanecarboxamido)-1-(methylamino)-2,7-naphthyridin-4-yl)cyclopent-3-ene-1-carboxamide

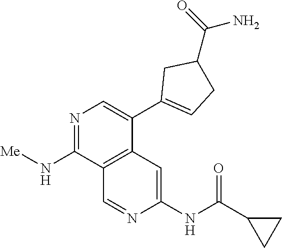 [SMILES: CNc1ncc(C2=CCC(C(N)=O)C2)c2cc(NC(=O)C3CC3)ncc12]

A solution of 3-(6-(cyclopropanecarboxamido)-1-(methylamino)-2,7-naphthyridin-4-yl)cyclopent-3-ene-1-carboxylic acid (220.0 mg; 0.624 mmol; 1.00 eq.), NH₄Cl (333.1 mg; 6.24 mmol; 10.00 eq.), DIPEA (242.3 mg; 1.872 mmol; 3.00 eq.) and HAM (356.1 mg; 0.936 mmol; 1.50 eq.) in DMF (2 mL) was stirred at room temperature for 1 hour under nitrogen atmosphere. The desired product was detected via LCMS. The resulting mixture was concentrated under reduced pressure and purified by flash chromatography on pre-packed C18 column using 20-100% of MeCN in water (10 mmol/L NH₄HCO₃) as eluent to provide the crude product. The product was purified by flash chromatography on silica gel column using 3-7% of MeOH in CH₂Cl₂ as eluent to provide 3-(6-(cyclopropanecarboxamido)-1-(methylamino)-2,7-naphthyridin-4-yl)cyclopent-3-ene-1-carboxamide as an off-white solid (75.0 mg, 34.2%). LCMS (ESI) m/z 352.2, [M+H]⁺.

### Step 5: N-(5-(4-(1H-1,2,4-triazol-3-yl)cyclopent-1-en-1-yl)-8-(methylamino)-2,7-naphthyridin-3-yl)cyclopropanecarboxamide

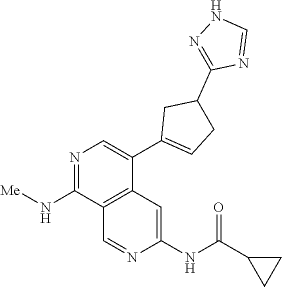 [SMILES: CNc1ncc(C2=CCC(c3nc[nH]n3)C2)c2cc(NC(=O)C3CC3)ncc12]

A solution of 3-(6-(cyclopropanecarboxamido)-1-(methylamino)-2,7-naphthyridin-4-yl)cyclopent-3-ene-1-carboxamide (75.0 mg; 0.213 mmol; 1.00 eq.) and DMF-DMA (254.1 mg; 2.130 mmol; 10.00 eq.) was stirred at 95° C. for 1 hour. The resulting mixture was concentrated under reduced pressure. The residue was redissolved in EtOH (1 mL) and named solution A. Another solution of AcOH (2 mL) in EtOH (8 mL) was allowed to cool down to 0° C., to this solution were added hydrazine hydrate (80%) (133.1 mg; 2.130 mmol; 10.00 eq.) and solution A dropwise at −10° C. The resulting mixture was stirred at room temperature overnight. The desired product was detected via LCMS. The EtOH was removed under reduced pressure. The remaining mixture was purified by flash chromatography on pre-packed C18 column using 20-100% of MeCN in water (10 mmol/L NH₄HCO₃) as eluent to provide N=(5-(4-(1H-1,2,4-triazol-3-yl)cyclopent-1-en-1-yl)-8-(methylamino)-2,7-naphthyridin-3-yl)cyclopropanecarboxamide as a yellow solid (7.6 mg, 9.3%). LCMS (ESI) m/z 376.2, [M+H]⁺.

### Example 834 and 835: Synthesis of 11′-(5-(4-(1-methyl-1H-1,2,4-triazol-3-yl)cyclopent-1-en-1-yl)-8-(methylamino)-2,7-naphthyridin-3-yl)cyclopropanecarboxamide (Example 834) and N-(5-(4-(1-methyl-1H-1,2,4-triazol-5-yl)cyclopent-1-en-1-yl)-8-(methylamino)-2,7-naphthyridin-3-yl)cyclopropanecarboxamide (Example 835)

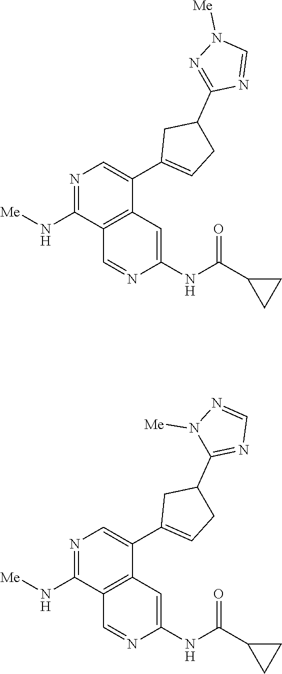 [SMILES: CNc1ncc(C2=CCC(c3ncn(C)n3)C2)c2cc(NC(=O)C3CC3)ncc12 ; CNc1ncc(C2=CCC(c3ncnn3C)C2)c2cc(NC(=O)C3CC3)ncc12]

To a stirred solution of N-(5-(4-(1H-1,2,4-triazol-3-yl)cyclopent-1-en-1-yl)-8-(methylamino)-2,7-naphthyridin-3-yl)cyclopropanecarboxamide (Example 833) (50.0 mg; 0.133 mmol; 1.00 eq.) and K₂CO₃ (27.6 mg; 0.200 mmol; 1.50 eq.) in DMF (1 mL) was added MeI (20.7 mg; 0.146 mmol; 1.10 eq.) at 0° C. The mixture was stirred at room temperature for 12 hours under nitrogen atmosphere. The desired product was detected via LCMS. The resulting mixture was diluted with EtOAc (50 mL) and washed with brine (3×5 mL). The organic layer was dried over anhydrous Na₂SO₄, concentrated under vacuum, and purified by flash chromatography on silica gel column using 0-10% of MeOH in CH₂Cl₂ as eluent to provide N-(5-(4-(1-methyl-1H-1,2,4-triazol-3-yl)cyclopent-1-en-1-yl)-8-(methylamino)-2,7-naphthyridin-3-yl)cyclopropanecarboxamide (Example 834) as a yellow solid (9.5 mg, 18.1%) and N-(5-(4-(1-methyl-1H-1,2,4-triazol-5-yl)cyclopent-1-en-1-yl)-8-(methylamino)-2,7-naphthyridin-3-yl)cyclopropanecarboxamide (Example 835) as a yellow solid (10.7 mg, 20.4%). LCMS (ESI) m/z 390.2, [M+H]⁺.

HNMR for Example 834: ¹H NMR (400 MHz, DMSO-d₆) δ 10.96 (s, 1H), 9.33 (s, 1H), 8.65 (s, 1H), 8.34 (s, 1H), 8.02-7.84 (m, 2H), 6.02-5.90 (m, 1H), 3.82 (s, 3H), 3.71-3.61 (m, 1H), 3.13-3.01 (m, 2H), 3.01-2.81 (m, 5H), 2.12-1.98 (m, 1H), 0.92-0.71 (m, 4H).

### Examples 835A and 836: Synthesis of N-(5-(1-(2-(1-methyl-1H-1,2,4-triazol-3-yl)ethyl)-1H-pyrazol-3-yl)-8-(methylamino)-2,7-naphthyridin-3-yl)cyclopropanecarboxamide (Example 835A) and N-(5-(1-(2-(1-methyl-1H-1,2,4-triazol-5-yl)ethyl)-1H-pyrazol-3-yl)-8-(methylamino)-2,7-naphthyridin-3-yl)cyclopropanecarboxamide (Example 836)

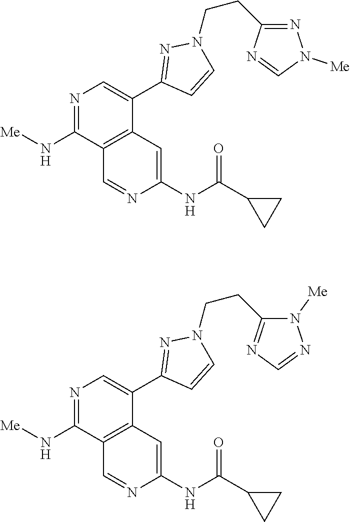 [SMILES: CNc1ncc(-c2ccn(CCc3ncn(C)n3)n2)c2cc(NC(=O)C3CC3)ncc12 ; CNc1ncc(-c2ccn(CCc3ncnn3C)n2)c2cc(NC(=O)C3CC3)ncc12]

### Step 1: 3-(3-bromo-1H-pyrazol-1-yl)propanenitrile

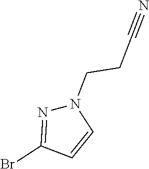

To a solution of 3-bromo-1H-pyrazole (2.00 g; 13.61 mmol; 1.00 eq.) in MeCN (30 mL) were added 3-bromopropanenitrile (2.70 g; 20.41 mmol; 1.50 eq.) and K₂CO₃ (3.80 g; 27.22 mmol; 2.00 eq.). The resulting mixture was stirred at 60° C. for 16 hours under nitrogen atmosphere. The desired product was detected via LCMS. After the reaction was completed, the mixture was cooled to room temperature and diluted with water (100 mL). The resulting mixture was extracted with ethyl acetate (3×30 mL). The combined organic layer was washed with brine, dried over anhydrous sodium sulfate and filtered. The filtrate was concentrated under vacuum to afford 3-(3-bromo-1H-pyrazol-1-yl)propanenitrile as a yellow oil (2.70 g, 99.0%). LCMS (ESI) m/z 200.0, [M+H]⁺.

### Step 2: 3-(2-(3-bromo-1H-pyrazol-1-yl)ethyl)-1H-1,2,4-triazole

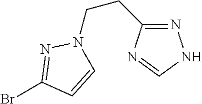 [SMILES: Brc1ccn(CCc2nc[nH]n2)n1]

To a solution of 3-(3-bromo-1H-pyrazol-1-yl)propanenitrile (2.70 g; 13.50 mmol; 1.00 eq.) in MeOH (40.0 mL) was added formohydrazide (2.00 g; 33.74 mmol; 2.50 eq.) and K₂CO₃ (2.40 g; 17.55 mmol; 1.30 eq.). The resulting mixture was stirred at 60° C. for 16 hours under nitrogen atmosphere. The desired product was detected via LCMS. After the reaction was completed, the mixture was cooled to room temperature and concentrated under vacuum. The residue was purified by flash chromatography on silica gel column using 1-9% of MeOH in CH₂Cl₂ as eluent to provide 3-(2-(3-bromo-1H-pyrazol-1-yl)ethyl)-1H-1,2,4-triazole as a light brown oil (594.0 mg, 18.5%). LCMS (ESI) m/z 242.0, [M+H]⁺.

### Step 3: 3-(2-(3-bromo-1H-pyrazol-1-yl)ethyl)-1-methyl-1H-1,2,4-triazole and 5-(2-(3-bromo-1H-pyrazol-1-yl)ethyl)-1-methyl-1H-1,2,4-triazole

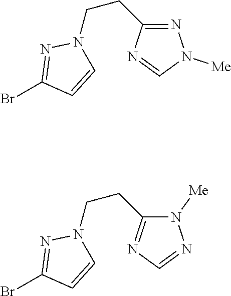 [SMILES: Cn1cnc(CCn2ccc(Br)n2)n1 ; Cn1ncnc1CCn1ccc(Br)n1]

To a solution of 3-(2-(3-bromo-1H-pyrazol-1-yl)ethyl)-1H-1,2,4-triazole (594.0 mg; 2.45 mmol; 1.00 eq.) in DMF (10 mL) was added iodomethane (348.3 mg; 2.45 mmol; 1.00 eq.) and K₂CO₃ (508.7 mg; 3.68 mmol; 1.50 eq.). The resulting mixture was stirred at room temperature for 4 hours. The desired product was detected via LCMS. After the reaction was completed, the mixture was diluted with water (80 mL). The resulting mixture was extracted with ethyl acetate (3×30 mL). The combined organic layer was washed with brine, dried over anhydrous sodium sulfate and filtered. The filtrate was concentrated under vacuum. The residue was purified by flash chromatography on silica gel column using 20-80% of EtOAc in petroleum ether as eluent to provide a mixture of 3-(2-(3-bromo-1H-pyrazol-1-yl)ethyl)-1-methyl-1H-1,2,4-triazole and 5-(2-(3-bromo-1H-pyrazol-1-yl)ethyl)-1-methyl-1H-1,2,4-triazole as a yellow oil (375.0 mg, 60.0%). LCMS (ESI) m/z 256.0, [M+H]⁺.

### Step 4: N-(5-(1-(2-(1-methyl-1H-1,2,4-triazol-3-yl)ethyl)-1H-pyrazol-3-yl)-8-(methylamino)-2,7-naphthyridin-3-yl)cyclopropanecarboxamide and N-(5-(1-(2-(1-methyl-1H-1,2,4-triazol-5-yl)ethyl)-1H-pyrazol-3-yl)-8-(methylamino)-2,7-naphthyridin-3-yl)cyclopropanecarboxamide

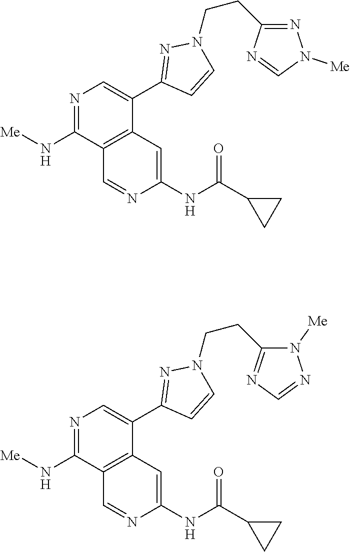 [SMILES: CNc1ncc(-c2ccn(CCc3ncn(C)n3)n2)c2cc(NC(=O)C3CC3)ncc12 ; CNc1ncc(-c2ccn(CCc3ncnn3C)n2)c2cc(NC(=O)C3CC3)ncc12]

To a solution of 3-(2-(3-bromo-1H-pyrazol-1-yl)ethyl)-1-methyl-1H-1,2,4-triazole and 5-(2-(3-bromo-1H-pyrazol-1-yl)ethyl)₁-methyl-1H-1,2,4-triazole (200.0 mg; 0.78 mmol; 1.00 eq.) in a mixture solvent of 1,4-dioxane/water (5:1, 4.8 mL) were added N-(8-(methylamino)-5-(4,4,5,5-tetramethyl-1,3,2-dioxaborolan-2-yl)-2,7-naphthyridin-3-yl)cyclopropanecarboxamide (287.6 mg; 0.78 mmol; 1.00 eq.), XPhos Pd G₃ (66.1 mg; 0.08 mmol; 0.10 eq.), XPhos (37.2 mg; 0.156 mmol; 0.10 eq.) and K₃PO₄ (497.3 mg; 2.34 mmol; 2.00 eq.). The resulting mixture was stirred at 60° C. for 1 hour under nitrogen atmosphere. The desired product was detected via LCMS. The mixture was concentrated under vacuum and purified by flash chromatography on silica gel column using 1-9% of MeOH in CH₂Cl₂ as eluent to provide the mixture of the two products. The mixture product was repurified by reverse phase preparative HPLC (Prep-C18, 5 μM XBridge Prep Phenyl OBD column, 19×250 mm, waters; gradient elution of 15-35% MeCN in water over a 10 min period, where both water and MeCN contain 10 mmol/L NH₄HCO₃, flow rate: 25 mL/min, detector UV wavelength: 254 nm) to afford N-(5-(1-(2-(1-methyl-1H-1,2,4-triazol-3-yl)ethyl)-1H-pyrazol-3-yl)-8-(methylamino)-2,7-naphthyridin-3-yl)cyclopropanecarboxamide (Example 835A) as an off-white solid (19.6 mg, 5.9%) and N-(5-(1-(2-(1-methyl-1H-1,2,4-triazol-5-yl)ethyl)-1H-pyrazol-3-yl)-8-(methylamino)-2,7-naphthyridin-3-yl)cyclopropanecarboxamide (Example 836) as an off-white solid (31.5 mg, 9.6%). LCMS (ESI) m/z 418.2, [M+H]⁺.

### Example 837: Synthesis of N-(5-(5-methyl-1,2,4-oxadiazol-3-yl)-8-(methylamino)-2,7-naphthyridin-3-yl)cyclopropanecarboxamide

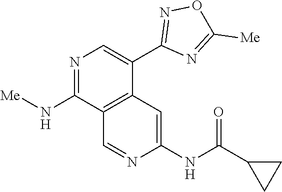 [SMILES: CNc1ncc(-c2noc(C)n2)c2cc(NC(=O)C3CC3)ncc12]

### Step 1: N-(5-cyano-8-(methylamino)-2,7-naphthyridin-3-yl)cyclopropanecarboxamide

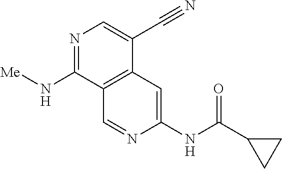 [SMILES: CNc1ncc(C#N)c2cc(NC(=O)C3CC3)ncc12]

A solution of N-(5-bromo-8-(methylamino)-2,7-naphthyridin-3-yl)cyclopropanecarboxamide (200.0 mg; 0.623 mmol; 1.00 eq.), Pd₂(dba)₃ (68.4 mg; 0.075 mmol; 0.12 eq.), S-Phos (61.6 mg; 0.150 mmol; 0.24 eq.) and Zn(CN)₂ (87.7 mg; 0.747 mmol; 1.20 eq.) in a mixture solvent of DMF/water (100:1, 4.04 mL) was stirred at 100° C. for 2.5 hours under nitrogen atmosphere. The desired product was detected via LCMS. The resulting mixture was diluted with EtOAc (20 mL). The resulting mixture was washed with water (3×4 mL). The organic layer was dried over anhydrous Na₂SO₄. After filtration, the filtrate was concentrated under reduced pressure. The residue was purified by flash chromatography on silica gel column using 2-10% of MeOH in CH₂Cl₂ to provide N-(5-cyano-8-(methylamino)-2,7-naphthyridin-3-yl)cyclopropanecarboxamide as a brown solid (100.0 mg, 59.1%). LCMS (ESI) m/z 268.1, [M+H]⁺.

### Step 2: N-(5-(N-hydroxycarbamimidoyl)-8-(methylamino)-2,7-naphthyridin-3-yl)cyclopropanecarboxamide

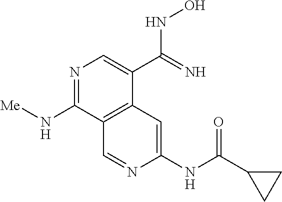 [SMILES: CNc1ncc(C(=N)NO)c2cc(NC(=O)C3CC3)ncc12]

To a solution of N-(5-cyano-8-(methylamino)-2,7-naphthyridin-3-yl)cyclopropanecarboxamide (100.0 mg; 0.374 mmol; 1.00 eq.) in a mixture solvent of DMSO/MeOH (1:10, 5.5 mL) was added hydroxylamine hydrochloride (38.7 mg; 0.557 mmol; 1.49 eq.) and NaHCO₃ (78.6 mg; 0.936 mmol; 2.50 eq.). The mixture was stirred at 60° C. for 16 hours. The desired product was detected via LCMS. The resulting mixture was concentrated under vacuum to afford N-(5-(N-hydroxycarbamimidoyl)-8-(methylamino)-2,7-naphthyridin-3-yl)cyclopropanecarboxamide as a yellow solid (180 mg, crude). LCMS (ESI) m/z 301.1, [M+H]⁺.

### Step 3: N-(5-(5-methyl-1,2,4-oxadiazol-3-yl)-8-(methylamino)-2,7-naphthyridin-3-yl)cyclopropanecarboxamide

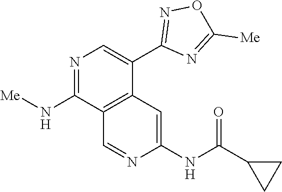 [SMILES: CNc1ncc(-c2noc(C)n2)c2cc(NC(=O)C3CC3)ncc12]

To a solution of N-(5-(N-hydroxycarbamimidoyl)-8-(methylamino)-2,7-naphthyridin-3-yl)cyclopropanecarboxamide (100.0 mg; 0.333 mmol; 1.00 eq.) in DMSO (5 mL) were added NaOH (20.0 mg; 0.500 mmol; 1.50 eq.) and methyl acetate (37.0 mg; 0.499 mmol; 1.50 eq.). The mixture was stirred at room temperature for 16 hours. The desired product was detected via LCMS. The mixture was purified by flash chromatography on pre-packed C18 column using 20-50% of MeCN in water (0.05% formic acid) as eluent to afford the crude product. The crude product was purified by reverse phase preparative HPLC (Prep-C18, 5 μM XSelect CSH OBD column, 19×250 mm, waters; gradient elution of 47-57% MeOH in water over a 10 min period, where both water and MeOH contain 10 mmol/L NH₄HCO₃, flow rate: 20 mL/min, detector UV wavelength: 254 nm) to provide N-(5-(5-methyl-1,2,4-oxadiazol-3-yl)-8-(methylamino)-2,7-naphthyridin-3-yl)cyclopropanecarboxamide as a white solid (4.1 mg, 3.8%). LCMS (ESI) m/z 325.1, [M+H]⁺.

### Example 838: Synthesis of N-(8-(methylamino)-5-(3-(methylsulfonyl)phenethyl)-2,7-naphthyridin-3-yl)cyclopropanecarboxamide

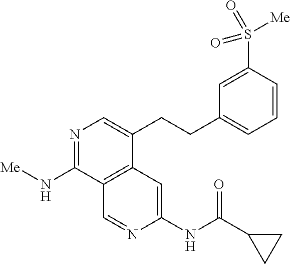 [SMILES: CNc1ncc(CCc2cccc(S(C)(=O)=O)c2)c2cc(NC(=O)C3CC3)ncc12]

### Step 1: 1-(methylsulfonyl)-3-vinylbenzene

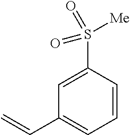

To a stirred solution of 1-bromo-3-methanesulfonylbenzene (500.0 mg; 2.127 mmol; 1.00 eq.) and 4,4,5,5-tetramethyl-2-vinyl-1,3,2-dioxaborolane (362.3 mg; 2.350 mmol; 1.10 eq.) in a mixture solvent of 1,4-dioxane/water (5:1, 6 mL) were added Pd(OAc)₂ (24.1 mg; 0.107 mmol; 0.05 eq.), S-Phos (87.2 mg, 0.212 mmol; 0.10 eq.) and K₃PO₄ (1.36 g, 6.402 mmol; 3.00 eq.). The resulting mixture was stirred at 80° C. for 14 hours under nitrogen atmosphere. The desired product was detected via LCMS. The mixture was allowed to cool down to room temperature, diluted with water and extracted with EtOAc (3×20 mL). The combined organic layers were washed with brine (3×20 mL) and dried over anhydrous Na₂SO₄. After filtration, the filtrate was concentrated under reduced pressure. The residue was purified by flash chromatography on silica gel column using 20-30% of EtOAc in petroleum ether as eluent to provide 1-(methylsulfonyl)-3-vinylbenzene as a yellow oil (293.0 mg, 75.6%). LCMS (ESI) m/z 183.0, [M+H]⁺.

### Step 2: (E)-N-(8-(methylamino)-5-(3-(methylsulfonyl)styryl)-2,7-naphthyridin-3-yl)cyclopropanecarboxamide

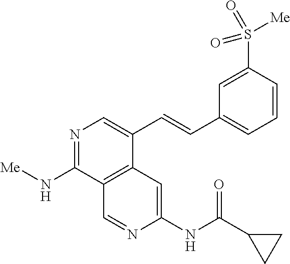 [SMILES: CNc1ncc(/C=C/c2cccc(S(C)(=O)=O)c2)c2cc(NC(=O)C3CC3)ncc12]

To a stirred solution of 1-(methylsulfonyl)-3-vinylbenzene (56.8 mg; 0.312 mmol; 1.00 eq.) and N-[5-bromo-8-(methyl amino)-2,7-naphthyridin-3-yl]cyclopropanecarboxamide (100.0 mg; 0.311 mmol; 1.00 eq.) in DMF (1 mL) were added Pd(PPh₃)₂Cl₂ (22.2 mg; 0.031 mmol; 0.10 eq.) and Na₂CO₃ (66.1 mg; 0.623 mmol; 2.00 eq.). The resulting mixture was stirred at 100° C. for 12 hours under nitrogen atmosphere. The desired product was detected via LCMS. The mixture was allowed to cool down to room temperature, diluted with water (10 mL) and extracted with EtOAc (3×20 mL). The combined organic layers were washed with brine (3×20 mL) and dried over anhydrous Na₂SO₄. After filtration, the filtrate was concentrated under reduced pressure. The residue was purified by flash chromatography on silica gel column using 5-8% of MeOH in CH₂Cl₂ as eluent to provide (E)-N-(8-(methylamino)-5-(3-(methylsulfonyl)styryl)-2,7-naphthyridin-3-yl)cyclopropanecarboxamide as a yellow solid (90.1 mg, 68.3%). LCMS (ESI) m/z 423.1, [M+H]⁺.

### Step 3: N-(8-(methylamino)-5-(3-(methylsulfonyl)phenethyl)-2,7-naphthyridin-3-yl)cyclopropanecarboxamide

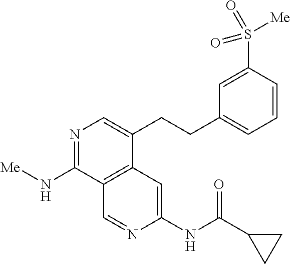 [SMILES: CNc1ncc(CCc2cccc(S(C)(=O)=O)c2)c2cc(NC(=O)C3CC3)ncc12]

To a solution of (L)-N-(8-(methylamino)-5-(3-(methylsulfonyl)styryl)-2,7-naphthyridin-3-yl)cyclopropanecarboxamide (80.2 mg; 0.1.82 mmol; 1.00 eq.) in MeOH (4 mL) was added 10% Pd/C (80.0 mg; 100% w/w) under nitrogen atmosphere. The mixture was hydrogenated at room temperature for 12 hours under hydrogen atmosphere (2 atm). The desired product was detected via LCMS. The mixture was filtered and concentrated under reduced pressure. The residue was purified by flash chromatography on silica gel column using 5-8% of MeOH in CH₂Cl₂ as eluent to provide the crude product. The crude product was purified by flash chromatography on pre-packed C18 column using 20-60% of MeCN in water (10 mmol/L NH₄HCO₃) to provide N-(8-(methylamino)-5-(3-(methylsulfonyl)phenethyl)-2,7-naphthyridin-3-yl)cyclopropanecarboxamide as a off-white solid (5.8 mg, 7.4%). LCMS (ESI) m/z 425.2, [M+H]⁺.

### Example 839: Synthesis of N-(8-(methylanilino)-5-(4-(methylsulfonyl)phenethyl)-2,7-naphthyridin-3-yl)cyclopropanecarboxamide

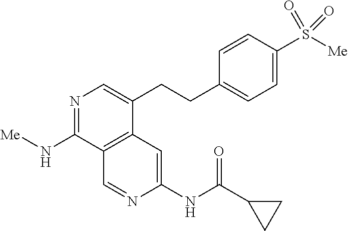 [SMILES: CNc1ncc(CCc2ccc(S(C)(=O)=O)cc2)c2cc(NC(=O)C3CC3)ncc12]

### Step 1: 1-(methylsulfonyl)-4-vinylbenzene

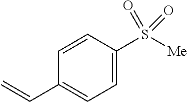

To a solution of 1-bromo-4-(methylsulfonyl)benzene (200.0 mg; 0.851 mmol; 1.00 eq.) in a mixture solvent of 1,4-dioxane/water (5:1, 6 mL) were added S-Phos (34.9 mg; 0.085 mmol; 0.10 eq.), K₃PO₄ (541.7 mg; 2.553 mmol; 3.00 eq.), Pd(OAc)₂ (9.5 mg; 0.043 mmol; 0.05 eq.) and 4,4,5,5-tetramethyl-2-vinyl-1,3,2-dioxaborolane (144.1 mg; 0.936 mmol; 1.10 eq.) at room temperature under nitrogen atmosphere. The resulting mixture was stirred at 90° C. for 2 hours. The desired product was detected via LCMS. The mixture was allowed to cool down to room temperature, concentrated under reduced pressure and purified by flash chromatography on silica gel column using 5-8% of MeOH in CH₂Cl₂ as eluent to provide 1-(methylsulfonyl)-4-vinylbenzene as a brown solid (110.0 mg, 70.9%). LCMS (ESI) m/z 183.0, [M+H]⁺.

### Step 2: (E)-N-(8-(methylamino)-5-(4-(methylsulfonyl)styryl)-2,7-naphthyridin-3-yl)cycloprapanecarboxamide

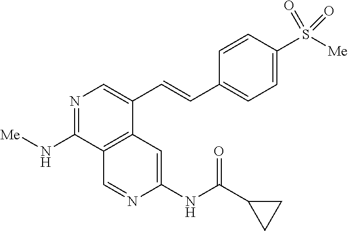 [SMILES: CNc1ncc(/C=C/c2ccc(S(C)(=O)=O)cc2)c2cc(NC(=O)C3CC3)ncc12]

To a solution of N-(5-bromo-8-(methylamino)-2,7-naphthyridin-3-yl)cyclopropanecarboxamide (120.0 mg; 0.373 mmol; 1.00 eq.) in DMF (1 mL) were added Pd(PPh₃)₂Cl₂ (27.5 mg; 0.037 mmol; 0.10 eq.), Na₂CO₃ (79.2 mg; 0.747 mmol; 2.00 eq.) and 1-(methylsulfonyl)-4-vinylbenzene (68.1 mg; 0.373 mmol; 1.00 eq.) at room temperature. The resulting mixture was stirred at 90° C. for 2 hours under nitrogen atmosphere. The desired product was detected via LCMS. The mixture was allowed to cool down to room temperature, concentrated under reduced pressure and purified by flash chromatography on silica gel column using 1-9% of MeOH in CH₂Cl₂ as eluent to provide (E)N-(8-(methylamino)-5-(4-(methylsulfonyl)styryl)-2,7-naphthyridin-3-yl)cyclopropanecarboxamide as a yellow solid (62.0 mg, 39.9%). LCMS (ESI) m/z 423.1, [M+H]⁺.

### Step 3: N-(8-(methylamino)-5-(4-(methylsulfonyl)phenethyl)-2,7-naphthyridin-3-yl)cyclopropanecarboxamide

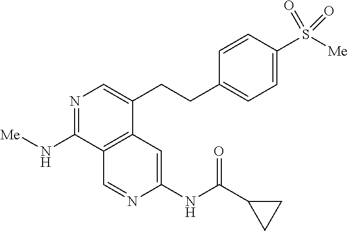 [SMILES: CNc1ncc(CCc2ccc(S(C)(=O)=O)cc2)c2cc(NC(=O)C3CC3)ncc12]

To a solution of (E)-N-(8-(methylamino)-5-(4-(methylsulfonyl)styryl)-2,7-naphthyridin-3-yl)cyclopropanecarboxamide (60.0 mg; 0.142 mmol; 1.00 eq.) in MeOH (3 mL) was added 10% Pd/C (30 mg; 1.00% w/w) under nitrogen atmosphere. The resulting mixture was hydrogenated at room temperature for 2 hours under hydrogen atmosphere (2 atm). The desired product was detected via LCMS. The mixture was filtered, concentrated under reduced pressure and purified by flash chromatography on silica gel column using 2-8% of MeOH in CH₂Cl₂ as eluent to provide the crude product. The crude product was repurified by flash chromatography on pre-packed C18 column using 20-60% of MeCN in water (10 mmol/L NH₄HCO₃) to afford N-(8-(methylamino)-5-(4-(methylsulfonyl)phenethyl)-2,7-naphthyridin-3-yl)cyclopropanecarboxamide as a white solid (12.6 mg, 20.7%). LCMS (ESI) m/z 425.2, [M+H]⁺. ¹H NMR (400 MHz, DMSO-d₆) δ 11.01 (s, 1H), 9.32 (s, 1H), 8.42 (s, 1H), 7.88-7.71 (m, 4H), 7.52 (d, J=7.9 Hz, 2H), 3.19 (s, 3H), 2.99 (t, J=8.2 Hz, 4H), 2.93 (d, J=4.4 Hz, 3H), 2.13-2.02 (m, 1H), 0.94-0.80 (m, 4H).

### Example 840: Synthesis of N-(8-(methylamino)-5-(pyrrolidine-1-carbonyl)-2,7-naphthyridin-3-yl)cyclopropanecarboxamide

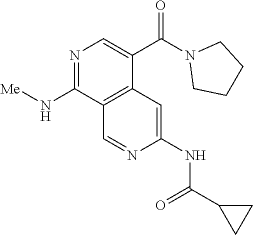 [SMILES: CNc1ncc(C(=O)N2CCCC2)c2cc(NC(=O)C3CC3)ncc12]

To a solution of 6-(cyclopropanecarboxamido)-1-(methylamino)-2,7-naphthyridine-4-carboxylic acid (100.0 mg; 0.341 mmol; 1.00 eq.) and ((3H-[1,2,3]triazolo[4,5-b]pyridin-3-yl)oxy)tri(pyrrolidin-1-yl)phosphonium hexafluorophosphate(V) (218.0 mg; 0.410 mmol; 1.20 eq.) in DMA (3 mL) were added DIEA (136.0 mg; 1.051 mmol; 3.01 eq.) and DMAP (9.0 mg; 0.071 mmol; 0.21 eq.) at room temperature under nitrogen atmosphere. The resulting mixture was stirred at 45° C. for 3 hours. The desired product was detected via LCMS. The resulting mixture concentrated under vacuum and purified by flash chromatography on pre-packed C18 column using 20-60% of MeCN in water (10 mmol/L NH₄HCO₃) to afford N-(8-(methylamino)-5-(pyrrolidine-1-carbonyl)-2,7-naphthyridin-3-yl)cyclopropanecarboxamide as a white solid (11.4 mg, 9.4%). LCMS (ESI) m/z 340.2, [M+H]⁺.

### Example 841: Synthesis of N-(5-(imidazo[1,2-a]pyrimidin-2-yl)-8-(methylamino)-2,7-naphthyridin-3-yl)cyclopropanecarboxamide

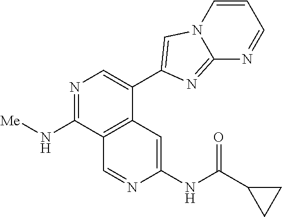 [SMILES: CNc1ncc(-c2cn3cccnc3n2)c2cc(NC(=O)C3CC3)ncc12]

### Step 1: N-(5-(1-ethoxyvinyl)-8-(methylamino)-2,7-naphthyridin-3-yl)cyclopropanecarboxamide

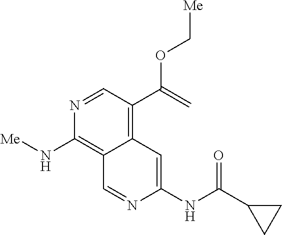 [SMILES: C=C(OCC)c1cnc(NC)c2cnc(NC(=O)C3CC3)cc12]

To a solution of N-(5-bromo-8-(methylamino)-2,7-naphthyridin-3-yl)cyclopropanecarboxamide (1.00 g; 3.114 mmol; 1.00 eq.) and tributyl(1-ethoxyvinyl)stannane (1.35 g; 3.737 mmol; 1.20 eq.) in DMF (18 mL) was added Pd(PPh₃)₂Cl₂ (0.109 g; 0.155 mmol; 0.05 eq.). The resulting solution was stirred at 80° C. for 14 hours under nitrogen atmosphere. The desired product was detected via LCMS. The mixture was diluted with water (15 mL) and extracted with EtOAc (3×10 mL). The combined organic layers were washed with brine (2×10 mL) and dried over anhydrous Na₂SO₄. After filtration, the filtrate was concentrated under reduced pressure to afford N-(5-(1-ethoxyvinyl)-8-(methylamino)-2,7-naphthyridin-3-yl)cyclopropanecarboxamide as a light brown oil (4.0 g, crude). LCMS (ESI) m/z 313.2, [M+H]⁺.

### Step 2: 1V (5-acetyl-8-(methylamino)-2,7-naphthyridin-3-yl)cyclopropanecarboxamide

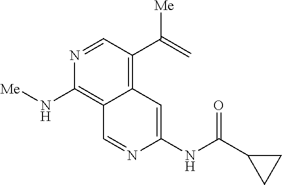 [SMILES: C=C(C)c1cnc(NC)c2cnc(NC(=O)C3CC3)cc12]

To a stirred solution of N-(5-(1-ethoxyvinyl)-8-(methylamino)-2,7-naphthyridin-3-yl)cyclopropanecarboxamide (1.40 g; 4.482 mmol; 1.00 eq.) in DMF (20 mL) was added HCl (3 M, 12.5 mL) dropwise at 0° C. The resulting mixture was stirred at room temperature overnight. The desired product was detected via LCMS. The resulting mixture was concentrated under reduced pressure and purified by flash chromatography on pre-packed C18 column using 30-50% of MeCN in water (10 mmol/L NH₄HCO₃) as eluent to provide N-(5-acetyl-8-(methylamino)-2,7-naphthyridin-3-yl)cyclopropanecarboxamide as an off-white solid (480.0 mg, 37.6%). LCMS (ESI) m/z 285.1, [M+H]⁺.

### Step 3: N-(5-(imidazo[1,2-a]pyrimidin-2-yl)-8-(methylamino)-2,7-naphthyridin-3-yl)cyclopropanecarboxamide

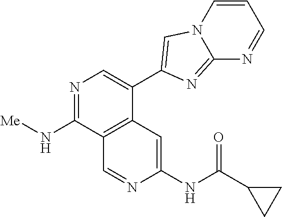 [SMILES: CNc1ncc(-c2cn3cccnc3n2)c2cc(NC(=O)C3CC3)ncc12]

Silica gel (5.00 g) was added to a mixture of CeCl₃·7H₂O (1.12 g; 3 mmol) and NaI (0.45 g; 3 mmol) in MeCN (100 mL). The mixture was stirred at room temperature overnight. The MeCN was removed by rotary evaporation to afford CeCl₃·7H₂O/NaI that was stored in a bottle at room temperature. To a mixture of N-(5-acetyl-8-(methylamino)-2,7-naphthyridin-3-yl)cyclopropanecarboxamide (160.0 mg; 0.563 mmol; 1.00 eq.) and pyrimidin-2-amine (53.5 mg; 0.563 mmol; 1.00 eq.) in EtOH (5 mL) was added CeCl₃·7H₂O/NaI (1.23 g; 0.563 mmol; 1.00 eq.). The resulting mixture was stirred at 135° C. for 2 hours under oxygen atmosphere under microwave radiation. The desired product was detected via LCMS. The mixture was allowed to cool down to room temperature. The solvent was concentrated under reduced pressure and purified by flash chromatography on silica gel column using 3-6% of MeOH in CH₂Cl₂ as eluent to provide the crude product. The crude product was repurified by Prep-Achiral-SFC (Column: XBridge Prep OBD C18 Column, 30×150 mm, 5 μm; Mobile Phase A: Water. (10 mmol/L NH₄HCO₃), Mobile Phase B: MeCN; Flow rate: 60 mL/min; Gradient: 13% B to 23% B in 10 min; Wave Length: 254 aura) to provide N=(5-(imidazo[1,2-a]pyrimidin-2-yl)-8-(methyl amino)-2,7-naphthyridin-3-yl)cyclopropanecarboxamide as a yellow solid (6.1 mg, 3.1%). LCMS (ESI) m/z 360.1, [M+H]⁺.

### Example 842: Synthesis of N-(8-(methylamino)-5-(5,6,7,8-tetrahydro-[1,2,4]triazolo[1,5-a]pyridin-2-yl)-2,7-naphthyridin-3-yl)cyclopropanecarboxamide

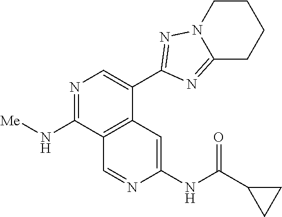 [SMILES: CNc1ncc(-c2nc3n(n2)CCCC3)c2cc(NC(=O)C3CC3)ncc12]

### Step 1: 5,6,7,8-tetrahydro-[1,2,4]triazolo[1,5-a]pyridin-2-amine

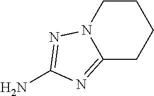

A solution of hydrazinecarboximidamide carbonate (5.00 g; 36.735 mmol; 1.00 eq.) and methyl 5-chloropentanoate (5.53 g; 36.735 mmol; 1.00 eq.) in ethane-1,2-diol (50 mL) was stirred at 110° C. for 16 hours under nitrogen atmosphere. The desired product was detected via LCMS. The mixture was allowed to cool down to room temperature and purified by flash chromatography on pre-packed C18 column using 20-60% of MeCN in water (10 mmol/L NH₄HCO₃) to afford 5,6,7,8-tetrahydro-[1,2,4]triazolo[1,5-a]pyridin-2-amine as an off-white solid (300 mg, 5.9%). LCMS (ESI) m/z 139.1, [M+H]⁺.

### Step 2: 2-chloro-5,6,7,8-tetrahydro-[1,2,4]triazolo[1,5-a]pyridine

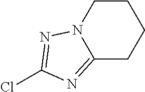

A solution of 5,6,7,8-tetrahydro-[1,2,4]triazolo[1,5-a]pyridin-2-amine (138.0 mg; 0.999 mmol; 1.00 eq.) and CuCl₂ (670.0 mg; 4.983 mmol; 4.99 eq.) in HCl (12 M, 20 mL) was stirred at 0° C. for 10 min under nitrogen atmosphere. To the above solution was added a solution of NaNO₂ (350.0 mg; 5.073 mmol; 5.08 eq.) in water (4 mL) dropwise at 0° C. The resulting mixture was stirred at room temperature for 0.5 hour. The desired product was detected via LCMS. The resulting mixture was concentrated under reduced pressure and purified by flash chromatography on pre-packed C18 column using 20-80% of MeCN in water (10 mmol/L NH₄HCO₃) to afford 2-chloro-5,6,7,8-tetrahydro-[1,2,4]triazolo[1,5-a]pyridine as a brown solid (20.0 mg, 1.2.7%). LCMS (ESI) m/z 158.0, [M+H]⁺.

### Step 3: N-(8-(methylamino)-5-(5,6,7,8-tetrahydra-[1,2,4]triazolo[1,5-a]pyridin-2-yl)-2,7-naphthyridin-3-yl)cyclopropanecarboxamide

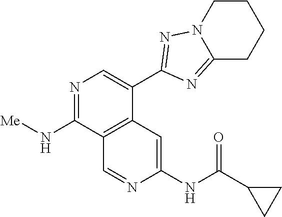 [SMILES: CNc1ncc(-c2nc3n(n2)CCCC3)c2cc(NC(=O)C3CC3)ncc12]

To a solution of 2-chloro-5,6,7,8-tetrahydro-[1,2,4]triazolo[1,5-c]pyridine (20 mg; 0.127 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (47.1 mg; 0.126 mmol; 1.00 eq.) in a mixture solvent of 1,4-dioxane/water (5:1, 1.8 mL) were added K₃PO₄ (55 mg; 0.259 mmol; 2.04 eq.), XPhos (12.5 mg; 0.026 mmol; 0.20 eq.) and XPhos Pd G₃ (11.0 mg; 0.013 mmol; 0.10 eq.) at room temperature under nitrogen atmosphere. The resulting mixture was stirred at 90° C. for 1 hour. The desired product was detected via LCMS. The mixture was allowed to cool down to room temperature, concentrated under reduced pressure and purified by flash chromatography on silica gel column using 5-10% of MeOH in CH₂Cl₂ as eluent to afford the crude product. The crude product was repurified by flash chromatography on pre-packed C18 column using 20-50% of MeCN in water (10 mmol/L NH₄HCO₃) to afford N-(8-(methylamino)-5-(5,6,7,8-tetrahydro-[1,2,4]triazolo[1,5-c]pyridin-2-yl)-2,7-naphthyridin-3-yl)cyclopropanecarboxamide as a white solid (5.8 mg, 12.3%). LCMS (ESI) m/z 364.2, [M+H]⁺. ¹H NMR (400 MHz, DMSO-d₆) δ 10.87 (s, 1H), 9.36 (s, 1H), 9.34 (s, 1H), 8.57 (s, 1H), 8.15-8.10 (m, 1H), 4.16 (t, J=6.0 Hz, 2H), 3.02 (d, J=4.4 Hz, 3H), 2.88 (t, J=6.0 Hz, 2H), 2.05-2.01 (m, 3H), 2.00-1.88 (m, 2H), 0.84-0.81 (m, 4H).

### Example 843: Synthesis of/V-(8-(methylamino)-5-(4,5,6,7-tetrahydrobenzo[d]oxazol-2-yl)-2,7-naphthyridin-3-yl)cyclopropanecarboxamide

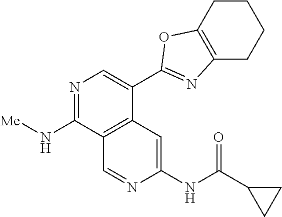 [SMILES: CNc1ncc(-c2nc3c(o2)CCCC3)c2cc(NC(=O)C3CC3)ncc12]

### Step 1: 6-(cyclopropanecarboxamido)-N-((1R,2R)-2-hydroxycyclohexyl)-1-(methylamino)-2,7-napthyridine-4-carboxamide

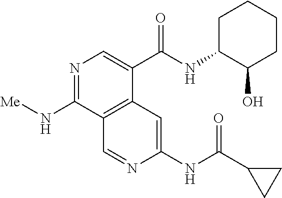 [SMILES: CNc1ncc(C(=O)N[C@@H]2CCCC[C@H]2O)c2cc(NC(=O)C3CC3)ncc12]

To a stirred solution of 6-cyclopropaneamido-1-(methylamino)-2,7-naphthyridine-4-carboxylic acid (200.0 mg; 0.699 mmol; 1.00 eq.), HOBt (141.0 mg; 1.048 mmol; 1.50 eq.) and EDCI.HCl (200.0 mg; 1.048 mmol; 1.50 eq.) in DMF (2 mL) was added (1R,2R)-2-aminocyclohexan-1-ol (160.0 mg; 1.398 mmol; 2.00 eq.) in portions at room temperature. The resulting mixture was stirred at room temperature for 6 hours under nitrogen atmosphere. The desired product was detected via LCMS. The mixture was concentrated under reduced pressure and purified by flash chromatography on pre-packed C18 column using 25-100% of MeCN in water (5 mmol/L NH₄HCO₃) as eluent to provide 6-(cyclopropanecarboxamido)-N-((1R,2R)-2-hydroxycyclohexyl)-1-(methylamino)-2,7-naphthyridine-4-carboxamide as an off-white solid (90.5 mg, 33.7%). LCMS (ESI) m/z 384.2, [M+H]⁺.

### Step 2: 6-(cyclopropanecarboxamido)-1-(methylamino)-N-(2-oxocyclohexyl)-2,7-naphthyridine-4-carboxamide

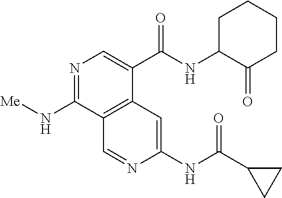 [SMILES: CNc1ncc(C(=O)NC2CCCCC2=O)c2cc(NC(=O)C3CC3)ncc12]

To a stirred solution of 6-(cyclopropanecarboxamido)-N-((1R,2R)-2-hydroxycyclohexyl)-1-(methylamino)-2,7-naphthyridine-4-carboxamide (108.0 mg; 0.282 mmol; 1.00 eq.) in a mixture solvent of DMF/CH₂Cl₂ (5:1, 18 mL) were added Dess-Martin (131 mg; 0.310 mmol; 1.10 eq.) and NaHCO₃(26.0 mg; 0.310 mmol; 1.10 eq.) in portions at 0° C. The mixture was stirred at room temperature for 5 hours under nitrogen atmosphere. The desired product was detected via LCMS. The reaction was quenched by the addition of saturated sodium bicarbonate solution (1 mL). The resulting mixture was diluted with CH₂Cl₂ (60 mL), washed with brine (2×5 mL), dried over anhydrous Na₂SO₄. After filtration, the filtrate was concentrated under reduced pressure. The residue was purified by flash chromatography on silica gel column using 3-1.0% of MeOH in CH₂Cl₂ as eluent to provide 6-(cyclopropanecarboxamido)-1-(methylamino)-N-(2-oxocyclohexyl)-2,7-naphthyridine-4-carboxamide as an off-white solid (35.0 mg, 32.5%). LCMS (ESI) m/z 382.2, [M+H]⁺.

### Step 3: N-(8-(methylamino)-5-(4,5,6,7-tetrahydrobenzo[d]oxazol-2-yl)-2,7-naphthyridin-3-yl)cyclopropanecarboxamide

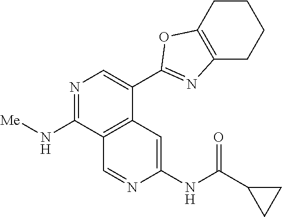 [SMILES: CNc1ncc(-c2nc3c(o2)CCCC3)c2cc(NC(=O)C3CC3)ncc12]

A solution of 6-(cyclopropanecarboxamido)-1-(methylamino)-N-(2-oxocyclohexyl)-2,7-naphthyridine-4-carboxamide (35.0 mg; 0.092 mmol; 1.00 eq.) and P₂O₅ (52.1 mg; 0.368 mmol; 4.00 eq.) in NMP (2 mL) was stirred at 1.40° C. for 6 ours under nitrogen atmosphere. The desired product was detected via LCMS. The mixture was purified by flash chromatography on pre-packed C18 column using 25-100% of MeCN in water (10 mmol/L NH₄HCO₃) as eluent to provide N-(8-(methylamino)-5-(4,5,6,7-tetrahydrobenzo[d]oxazol-2-yl)-2,7-naphthyridin-3-yl)cyclopropanecarboxamide as an off-white solid (12.6 mg, 37.5%). LCMS (ESI) m/z 364.2, [M+H]⁺. ¹H NMR (400 MHz, DMSO-d₆) δ 10.95 (s, 1H), 9.39 (s, 1H), 9.38 (s, 1H), 8.57 (s, 1H), 8.40-8.31 (m, 1H), 3.03 (d, J=4.4 Hz, 3H), 2.80-2.69 (m, 2H), 2.60-2.50 (m, 2H), 2.13-2.02 (m, 1H), 1.90-1.75 (m, 4H), 0.98-0.78 (m, 4H).

### Example 844: Synthesis of N-(5-(bicyclo[1.1.1]pentan-1-yl)-8-(methylamino)-2,7-naphthyridin-3-yl)cyclopropanecarboxamide

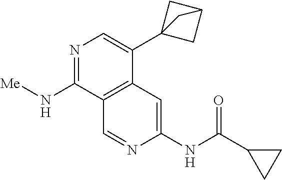 [SMILES: CNc1ncc(C23CC(C2)C3)c2cc(NC(=O)C3CC3)ncc12]

### Step 1: potassium bicyclo[1.1.1]pentan-1-yltrifluoroborate

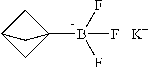

A solution of 2-(bicyclo[1.1.1]pentan-1-yl)-4,4,5,5-tetramethyl-1,3,2-dioxaborolane (70.0 g; 0.36 mmol; 1.00 eq.) and KHF₂ (141.0 mg; 1.80 mmol; 5.01 eq.) in a mixture solvent of MeOH/water (10:1, 3.3 mL) was stirred at room temperature for 3 hours under nitrogen atmosphere. The reaction was monitored by TLC. The suspension was filtered and washed with 2-methoxy-2-methylpropane (2×5 mL). The filtrate was collected and concentrated under vacuum, the residue was suspended in 2-methoxy-2-methylpropane (2 mL) and stirred at room temperature for 30 minutes. The precipitated solids were collected by filtration, which were added to acetone (5 mL) and stirred at room temperature for 30 minutes. The insoluble solid was filtered off, the filtrate was concentrated under vacuum to provide potassium bicyclo[1.1.1]pentan-1-yltrifluoroborate as a white solid (80 mg, crude).

### Step 2: N-(5-(bicyclo[1.1.1]pentan-1-yl)-8-(methylamino)-2,7-naphthyridin-3-yl)cyclopropanecarboxamide

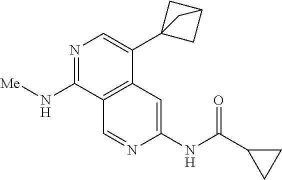 [SMILES: CNc1ncc(C23CC(C2)C3)c2cc(NC(=O)C3CC3)ncc12]

To a solution of N-(5-bromo-8-(methylamino)-2,7-naphthyridin-3-yl)cyclopropanecarboxamide (30.0 mg; 0.09 mmol; 1.00 eq.) and potassium bicyclo[1.1.1]pentan-1-yltrifluoroborate (21.0 mg; 0.18 mmol; 2.01 eq.) in DMF (0.9 mL) and nickel(2⁺) 2,2′-bipyridine dibromide (CAS: 46389-47-3) (5.0 mg; 0.01 mmol; 0.14 eq.) were added DBU (36.0 mg; 0.23 mmol; 2.53 eq.) and Ir[dF(CF₃)ppy]₂(bpy)PF₆ (CAS: 1092775-62-6) (7.0 mg; 0.01 mmol; 0.07 eq.). An inlet needle was inserted, and the atmosphere was exchanged for nitrogen. Then the reaction was stirred for 12 hours, irradiation was performed with a ring of blue LEDs (450 nm light). The desired product was detected via LCMS. The mixture was diluted with EtOAc (50 mL), washed with brine 3×5 mL), dried over anhydrous Na₂SO₄. The organic phase was concentrated in vacuo. The residue was purified by flash chromatography on silica gel column using 2-10% of MeOH in CH₂Cl₂ as eluent to provide the crude product. The product was re-purified by flash chromatography on pre-packed C18 column using 20-70% of MeCN in water (10 mmol/L NH₄HCO₃) to provide N-(5-(bicyclo[1.1.1]pentan-1-yl)-8-(methylamino)-2,7-naphthyridin-3-yl)cyclopropanecarboxamide as an off-white solid (2.4 mg, 4.3%). LCMS (ESI) m/z 309.2, [M+H]⁺.

### Example 845: Synthesis of N-(8-(methylamino)-5-(3-(pyridin-4-yl)bicyclo[1.1.1]pentan-1-yl)-2,7-naphthyridin-3-yl)cyclopropanecarboxamide

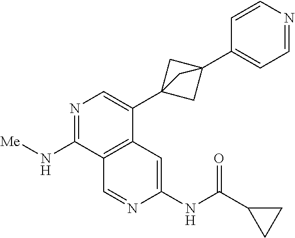 [SMILES: CNc1ncc(C23CC(c4ccncc4)(C2)C3)c2cc(NC(=O)C3CC3)ncc12]

### Step 1: 3-carboxy-2-(pyridin-1-ium-1-yl)propanoate

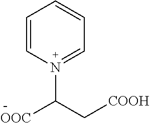

A solution of maleic acid (20.54 g; 176.987 mmol; 1.00 eq.) and pyridine (14.00 g; 176.987 mmol; 1.00 eq.) in water (250 mL) was stirred at 90° C. for 2 hours. After a crystalline solid appeared in the reaction mixture, acetic acid (12.75 g; 212.384 mmol; 1.20 eq.) was added. The suspension was kept under stirring at the same temperature for 24 hours. The reaction mixture was cooled down to room temperature, filtered and washed with MeOH and EtOAc. The resulting white solid was dried under reduced pressure by rotary evaporator for 4 hours. The 3-carboxy-2-(pyridin-1-ium-1-yl)propanoate was obtained as a white solid (18.01 g, crude). LCMS (ESI) m/z 196.1, [M+H]⁺.

### Step 2: 1-(1,4-diethoxy-1,4-dioxobutan-2-yl)pyridin-1-ium ethyl sulfate

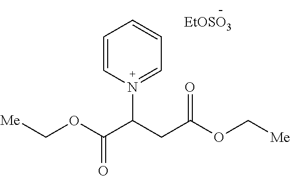

A solution of 3-carboxy-2-(pyridin-1-ium-1-yl)propanoate (9.70 g; 49.699 mmol; 1.00 eq.) in EtOH (250 mL) with concentrated sulfuric acid (5.25 mL) was stirred at 90° C. for 18 hours. The desired product was detected via LCMS. After the reaction was done, the solvent was evaporated by reduced pressure. The residue was diluted with dichloromethane/water (4:1, 250 mL) and extracted by dichloromethane. The combined organic phase was dried by Na₂SO₄, filtered and concentrated under reduced pressure. To the resulting colorless liquid was added anhydrous diethyl ether. (30 mL) and subsequently dried by reduced pressure. After the residual solvent was totally evaporated by reduced pressure, the 1-(1,4-diethoxy-1,4-dioxobutan-2-yl)pyridin-1-ium ethyl sulfate was obtained as a white solid (9.76 g, crude). The resulting product was used for next step without further purification. LCMS (ESI) m/z 252.1, [M]⁺.

### Step 3: 1-(1,4-diethoxy-1,4-dioxobutan-2-yl)-4-(3-(methoxycarbonyl)bicyclo[1.1.1]pentan-1-yl)pyridin-1-ium ethyl sulfate

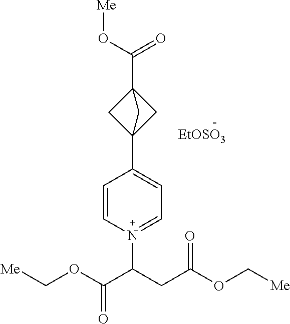

To a 250 mL round-bottom flask were added 1-(1,4-diethoxy-1,4-dioxobutan-2-yl)pyridin-1-ium ethyl sulfate (2.64 g; 7.000 mmol; 1.00 eq.), 3-(methoxycarbonyl)bicyclo[1.1.1]pentane-1-carboxylic acid (1.19 g; 7.000 mmol; 1.00 eq.), (NH₄)₂S₂O₈ (3.19 g; 14.000 mmol; 2.00 eq.) and AgNO₃ (236.6 mg; 1.400 mmol; 0.20 eq.), together with a mixture solvent of dichloroethane/water (1:1, 70 mL). The biphasic mixture was stirred at 50° C. for 2 hours under nitrogen atmosphere. The desired product was detected via LCMS. The reaction was diluted with dichloromethane (35 mL). The aqueous phase was extracted with dichloromethane (3×35 mL) and the combined organic layers were dried over Na₂SO₄, filtered and concentrated in vacuo to afford 1-(1,4-diethoxy-1,4-dioxobutan-2-yl)-4-(3-(methoxycarbonyl)bicyclo[1.1.1]pentan-1-yl)pyridin-1-ium ethyl sulfate (2.20 g, crude). The product was used for the next step without further purification. LCMS (ESI) m/z 376.2, [M]⁺.

### Step 4: methyl 3-(pyridin-4-yl)bicyclo[1.1.1]pentane-1-carboxylate

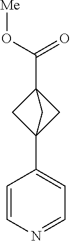 [SMILES: COC(=O)C12CC(c3ccncc3)(C1)C2]

To the solution of 1-(1,4-diethoxy-1,4-dioxobutan-2-yl)-4-(3-(methoxycarbonyl)bicyclo[1.1.1]pentan-1-yl)pyridin-1-ium ethyl sulfate (2.20 g, crude) in dichloromethane (70 mL) was added DBU (3.19 g; 21.000 mmol; 4.78 eq.) and the reaction mixture was stirred at room temperature for 0.5 hour under nitrogen atmosphere. The desired product was detected via LCMS. Upon the reaction completion, the reaction mixture was transferred to a separatory funnel containing 1 M NaOH for adjusting pH>10. The aqueous phase was extracted with dichloromethane (3×40 mL). The combined organic phase was washed with brine (20 mL) just once. The resulting organic layer was dried over Na₂SO₄, filtered, and concentrated in vacua. The residue was purified by flash chromatography on silica gel column using 0-40% of EtOAc in petroleum ether as eluent to provide methyl 3-(pyridin-4-yl)bicyclo[1.1.1]pentane-1-carboxylate as a light-yellow oil (300.8 mg, 33%). LCMS (ESI) m/z 204.1, [M+H]⁺.

### Step 5: 3-(pyridin-4-yl)bicyclo[1.1.1]pentane-1-carboxylic acid

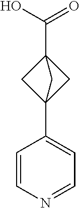 [SMILES: O=C(O)C12CC(c3ccncc3)(C1)C2]

To a stirred solution of methyl 3-(pyridin-4-yl)bicyclo[1.1.1]pentane-1-carboxylate (163.0 mg; 0.802 mmol; 1.00 eq.) in THF (6 mL) was added a solution of LiOH·H₂O (67.3 mg; 1.604 mmol; 2.00 eq) in water (2 mL) at room temperature. The resulting mixture was stirred at room temperature for 3 hours. The desired product was detected via LCMS. Then the mixture was acidified to pH=4-5 with 1 M HCl and concentrated under reduced pressure. The residue was purified by flash chromatography on pre-packed C18 column using 20-50% of MeCN in water (0.05% formic acid) as eluent to provide 3-(pyridin-4-yl)bicyclo[1.1.1]pentane-1-carboxylic acid as a white solid (104.6 mg, 68.9%). LCMS (ESI) m/z 188.1, [M−H]⁻.

### Step 6: 1,3-dioxoisoindolin-2-yl 3-(pyridin-4-yl)bicyclo[1.1.1]pentane-1-carboxylate

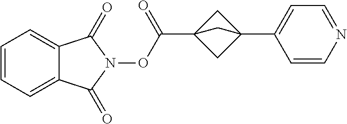 [SMILES: O=C1c2ccccc2C(=O)N1OC(=O)C12CC(c3ccncc3)(C1)C2]

To a stirred solution of 3-(pyridin-4-yl)bicyclo[1.1.1.]pentane-1-carboxylic acid (204.2 mg; 1.200 mmol; 1.00 eq.) in CH₂Cl₂ (6 mL) were added 2-hydroxyisoindoline-1,3-dione (215.3 mg; 1.320 mmol; 1.10 eq.), dicyclohexylmethanediimine (DCC) (272.3 mg; 1.320 mmol; 1.10 eq.) and DMAP (14.6 mg; 0.120 mmol; 1.00 eq.). The reaction was stirred at room temperature for 12 hours. The reaction was monitored by TLC (petroleum ether/EtOAc=2:1). Upon reaction completion, the mixture was filtered and concentrated in vacuo to yield 1,3-dioxoisoindolin-2-yl 3-(pyridin-4-yl)bicyclo[1.1.1]pentane-1-carboxylate as a white solid (297.3 mg, 74.1%).

### Step 7: N-(8-(methylamino)-5-(3-(pyridin-4-yl)bicyclo[1.1.1]pentan-1-yl)-2,7-naphthyridin-3-yl)cyclopropanecarboxamide

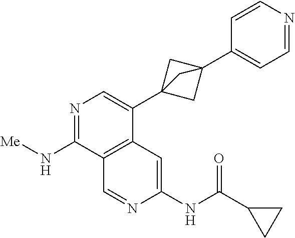 [SMILES: CNc1ncc(C23CC(c4ccncc4)(C2)C3)c2cc(NC(=O)C3CC3)ncc12]

To a 10 mL microwave tube equipped with a stir bar were added N-(5-bromo-8-((4-methoxybenzyl)(methyl)amino)-2,7-naphthyridin-3-yl)cyclopropanecarboxamide (24.3 mg; 0.055 mmol; 1.00 eq.), 1,3-dioxoisoindolin-2-yl 3-(pyridin-4-yl)bicyclo[1.1.1]pentane-1-carboxylate (36.8 mg; 0.110 mmol; 2.00 eq.), Ir[dF(CF₃)ppy]₂(bpy)PF₆ (CAS: 1092775-62-6) (5.5 mg; 0.006 mmol; 0.10 eq.), nickel(2⁺) 2,2′-bipyridine dibromide (CAS: 46389-47-3) (3.0 mg, 0.008 mmol; 0.1.5 eq.), Na₂CO₃ (11.6 mg; 0.110 mmol; 2.00 eq.) and DMA (0.6 mL). An inlet needle was inserted, and the atmosphere was exchanged for nitrogen. The reaction was stirred for 12 hours, irradiation was performed with a ring of blue LEDs (450 nm light). The desired product was detected via LCMS. Upon reaction completion. The mixture was purified by flash chromatography on pre-packed C₁₋₈ column using 0-100% of MeCN in water (10 mmol/L NH₄HCO₃) as eluent to provide N-(8-(methylamino)-5-(3-(pyridin-4-yl)bicyclo[1.1.1]pentan-1-yl)-2,7-naphthyridin-3-yl)cyclopropanecarboxamide as a white solid (1.6 mg, 7.5%). LCMS (ESI) m/z 386.2, [M+H]⁺.

### Example 846: Synthesis of N-(8-(ethylamino)-5-phenyl-2,7-naphthyridin-3-yl)cyclopropanecarboxamide

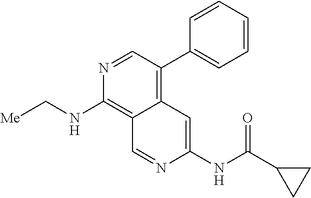 [SMILES: CCNc1ncc(-c2ccccc2)c2cc(NC(=O)C3CC3)ncc12]

### Step 1: 6-chloro-4-iodo-2,7-naphthyridin-1-ol

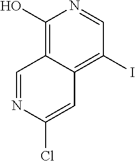 [SMILES: Oc1ncc(I)c2cc(Cl)ncc12]

A solution of 6-chloro-2,7-naphthyridin-1-ol (1 g; 5.53 mmol; 1.00 eq.) and 1-iodopyrrolidine-2,5-dione (1.87 g; 8.30 mmol; 1.50 eq.) in DMF (10 mL) was stirred at room temperature for 5 hours under nitrogen atmosphere. The desired product was detected via LCMS. The resulting mixture was added dropwise to water (50 mL) and stirred at room temperature for 30 minutes to precipitate solids. The precipitated solids were collected by filtration, the solids were washed with water and dried under reduced pressure to provide 6-chloro-4-iodo-2,7-naphthyridin-1-ol as a yellow solid (1.05 g, 61%). LCMS (ESI) m/z 306.9, [M+H]⁺.

### Step 2: 6-chloro-4-phenyl-2,7-naphthyridin-1(2H)-one

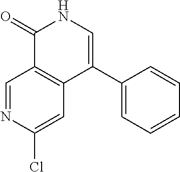 [SMILES: O=c1[nH]cc(-c2ccccc2)c2cc(Cl)ncc12]

A solution of 6-chloro-4-iodo-2,7-naphthyridin-1-ol (400 mg; 1.305 mmol; 1.00 eq.), phenylboronic acid (477 mg; 3.91 mmol; 3.00 eq.), Pd(PPh₃)₄ (151 mg; 0.131 mmol; 0.10 eq.) and Na₂CO₃ (748 mg; 7.06 mmol; 5.41 eq.) in a mixture solvent of DME/water (5:1, 12 mL) was stirred at 100° C. for 1 hour under nitrogen atmosphere. The desired product was detected via LCMS. The resulting mixture was concentrated under reduced pressure. The residue was purified flash chromatography on silica gel column using 2-10% of MeOH in CH₂Cl₂ as eluent to provide 6-chloro-4-phenyl-2,7-naphthyridin-1(2H)-one as a yellow solid (228 mg, 68%). LCMS (ESI) m/z 257.0, [M+H]⁺.

### Step 3: V-(8-hydroxy-5-phenyl-2,7-naphthyridin-3-yl)cyclopropanecarboxamide

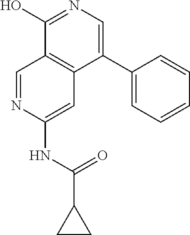 [SMILES: O=C(Nc1cc2c(-c3ccccc3)cnc(O)c2cn1)C1CC1]

A solution of 6-chloro-4-phenyl-2,7-naphthyridin-1(2H)-one (1.1 g; 4.29 mmol; 1.00 eq.), cyclopropanecarboxamide (1.46 g; 17.1 mmol; 4.00 eq.), Pd₂(dba)₃ (394.4 mg; 0.429 mmol; 0.1.0 eq.), XantPhos (495 mg; 0.859 mmol; 0.20 eq.) and Cs₂CO₃ (2.8 g; 8.58 mmol; 2.00 eq.) in dioxane (30 mL) was stirred at 130° C. overnight under nitrogen atmosphere. The desired product was detected via LCMS. The reaction was concentrated under reduced pressure and purified by flash chromatography on silica gel column using 3-15% of MeOH in CH₂Cl₂ to afford N-(8-hydroxy-5-phenyl-2,7-naphthyridin-3-yl)cyclopropanecarboxamide as a yellow solid (180 mg, 13%). LCMS (ESI) m/z 306.1, [M+H]⁺.

### Step 4: N-(8-chloro-5-phenyl-2,7-naphthyridin-3-yl)cyclopropanecarboxamide

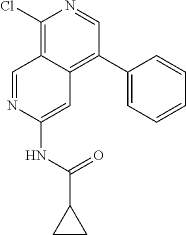 [SMILES: O=C(Nc1cc2c(-c3ccccc3)cnc(Cl)c2cn1)C1CC1]

A solution of N-(8-hydroxy-5-phenyl-2,7-naphthyridin-3-yl)cyclopropanecarboxamide (116 mg; 0.380 mmol; 1.00 eq.) in phosphorus oxychloride (3 mL) was stirred at 100° C. for 1 hour under nitrogen atmosphere. The desired product was detected via LCMS. The reaction was concentrated under reduced pressure. The resulting mixture was diluted with CH₂Cl₂ (50 mL) and washed with saturated NaHCO₃ solution. The organic layer was dried over Na₂SO₄, filtered and the filtrate was concentrated under reduced pressure. The residue was purified by flash chromatography on silica gel column using 20-50% of EtOAc in petroleum ether to afford N-(8-chloro-5-phenyl-2,7-naphthyridin-3-yl)cyclopropanecarboxamide as a white solid (50 mg, 40.7%). LCMS (ESI) m/z 324.1, [M+H]⁺.

### Step 5: N-(8-(ethylamino)-5-phenyl-2,7-naphthyridin-3-yl)cyclopropanecarboxamide

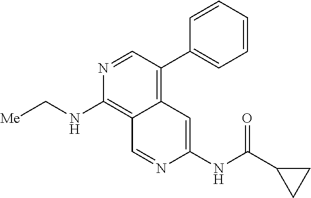 [SMILES: CCNc1ncc(-c2ccccc2)c2cc(NC(=O)C3CC3)ncc12]

To a solution of N-(8-chloro-5-phenyl-2,7-naphthyridin-3-yl)cyclopropanecarboxamide (50.0 mg; 0.1.54 mmol; 1.00 eq.), Pd-PEPPSI-IHeptCl 3-chloropyridine (15.0 mg; 0.015 mmol; 0.10 eq.) and Cs₂CO₃ (100.6 mg; 0.308 mmol; 2.00 eq.) in 1,4-dioxane (0.5 mL) was added ethylamine (2 M in THF, 0.23 mL) under nitrogen atmosphere. The reaction was stirred at 90° C. for 2 hours. The desired product was detected via LCMS. The reaction was concentrated under pressure and purified by flash chromatography on silica gel column using 1-9% of MeOH in CH₂Cl₂ as eluent to provide the crude product. The product was purified by flash chromatography on pre-packed C18 column using 10-70% of MeCN in water (10 mmol/L NH₄HCO₃) to provide N-(8-(ethylamino)-5-phenyl-2,7-naphthyridin-3-yl)cyclopropanecarboxamide as an off-white solid (29.1 mg, 56.3%). LCMS (ESI) m/z 333.2, [M+H]⁺.

### Example 847: Synthesis of N-(5-(4-(1,4-dimethyl-1H-pyrazol-5-yl)phenyl)-8-(methylamino)-2,7-naphthyridin-3-yl)cyclopropanecarboxamide

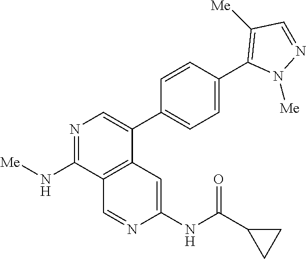 [SMILES: CNc1ncc(-c2ccc(-c3c(C)cnn3C)cc2)c2cc(NC(=O)C3CC3)ncc12]

### Step 1: 5-(4-bromophenyl)-1,4-dimethyl-1H-pyrazole

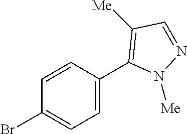 [SMILES: Cc1cnn(C)c1-c1ccc(Br)cc1]

To a solution of 1,4-dimethyl-5-(4,4,5,5-tetramethyl-1,3,2-dioxaborolan-2-yl)-1H-pyrazole (1.00 g; 4.502 mmol; 1.00 eq.) in a mixture solvent of DME/water (5:1, 12 mL) were added Pd(PPh₃)₄ (416.2 mg; 0.360 mmol; 0.10 eq.), Na₂CO₃ (749.2 mg; 7.068 mmol; 1.57 eq.) and 1-bromo-4-iodobenzene (789.7 mg; 2.791 mmol; 0.62 eq.). The reaction was stirred at 100° C. for 2 hours under nitrogen atmosphere. The desired product was detected via LCMS. The mixture was diluted with EtOAc (200 mL) and washed with brine (3×40 mL). The organic layer was dried over anhydrous sodium sulfate and concentrated under vacuum. The residue was purified by flash chromatography on silica gel column using 20-70% of EtOAc in petroleum ether as eluent to provide 5-(4-bromophenyl)-1,4-dimethyl-1H-pyrazole as white solid (320.0 mg, 17.7%). LCMS (ESI) m/z 251.0, [M+H]⁺.

### Step 2:1-(5-(4-(1,4-dimethyl-1H-pyrazol-5-yl)phenyl)-8-(methylamino)-2,7-naphthyridin-3-yl)cyclopropanecarbaxamide

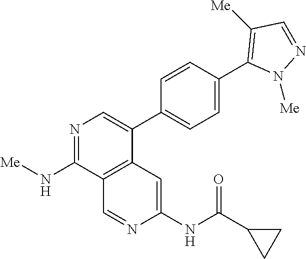 [SMILES: CNc1ncc(-c2ccc(-c3c(C)cnn3C)cc2)c2cc(NC(=O)C3CC3)ncc12]

To a solution of N-(8-(methylamino)-5-(4,4,5,5-tetramethyl-1,3,2-dioxaborolan-2-yl)-2,7-naphthyridin-3-yl)cyclopropanecarboxamide (150.0 mg; 0.407 mmol; 1.00 eq.) in a mixture solvent of 1,4-dioxane/water (5:1, 6 mL) was added XPhos Pd G₃ (34.4 mg; 0.041 mmol; 0.10 eq.), XPhos (19.4 mg; 0.041 mmol; 0.10 eq.), K₃PO₄ (259.0 mg; 1.220 mmol; 3.00 eq.) and 5-(4-bromophenyl)-1,4-dimethyl-1H-pyrazole (100.0 mg; 0.398 mmol; 0.98 eq.). The reaction was stirred at 90° C. for 1 hour under nitrogen atmosphere. The desired product was detected via LCMS. The resulting mixture was concentrated under reduced pressure and purified by flash chromatography on silica gel column using 2-10% of MeOH in CH₂Cl₂ as eluent to provide a crude product. The crude product was repurified by flash chromatography on pre-packed C18 column using 20-80% of MeCN in water (10 mmol/L NH₄HCO₃) as eluent to provide N-(5-(4-(1,4-dimethyl-1H-pyrazol-5-yl)phenyl)-8-(methyl amino)-2,7-naphthyridin-3-yl)cyclopropanecarboxamide as a yellow solid (32.6 mg, 19.4%). LCMS (ESI) m/z 413.2, [M+H]⁺. ¹H NMR (400 MHz, DMSO-d₆) δ 10.98 (s, 1H), 9.41 (s, 1H), 8.44 (s, 1H), 8.1.4-8.04 (m, 1H), 8.02 (s, 1H), 7.66-7.47 (m, 4H), 7.37 (s, 1H), 3.79 (s, 3H), 3.03 (d, J=4.4 Hz, 3H), 2.05 (s, 3H), 2.03-1.94 (m, 1H), 0.85-0.64 (m, 4H).

### Example 848: Synthesis of N-(8-(methylamino)-5-(4-(trifluoromethoxy)phenethyl)-2,7-naphthyridin-3-yl)cyclopropanecarboxamide

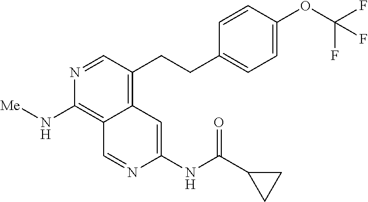 [SMILES: CNc1ncc(CCc2ccc(OC(F)(F)F)cc2)c2cc(NC(=O)C3CC3)ncc12]

### Step 1: (E)-N-(8-(methylamino)-5-(4-(trifluoromethoxy)styryl)-2,7-naphthyridin-3-yl)cyclopropanecarboxamide

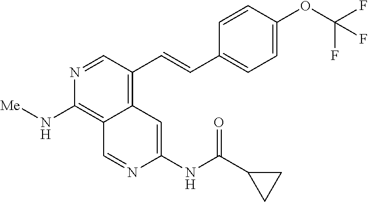 [SMILES: CNc1ncc(/C=C/c2ccc(OC(F)(F)F)cc2)c2cc(NC(=O)C3CC3)ncc12]

To a solution of N-(8-(methylamino)-5-vinyl-2,7-naphthyridin-3-yl)cyclopropanecarboxamide (100.0 mg; 0.373 mmol; 1.00 eq.) in DI (3 mL) was added Pd(PPh₃)₂Cl₂ (15.7 mg; 0.022 mmol; 0.06 eq.), Na₂CO₃ (50.1 mg; 0.474 mmol; 1.27 eq.) and 1-bromo-4-(trifluoromethoxy)benzene (84.4 mg; 0.351 mmol; 0.94 eq.). The reaction was stirred at 100° C. for 16 hours under nitrogen atmosphere. The desired product was detected via LCMS. The resulting mixture was diluted with EtOAc (50 mL) and washed with brine (3×6 mL). The organic layer was dried over anhydrous sodium sulfate and concentrated under vacuum. The residue was purified by flash chromatography on silica gel column using 2-10% of MeOH in CH₂Cl₂ as eluent to provide (E)-N-(8-(methylamino)-5-(4-(trifluoromethoxy)styryl)-2,7-naphthyridin-3-yl) cyclopropanecarboxamide as a white solid (20.0 mg, 12.5%). LCMS (ESI) m/z 429.1, [M+H]⁺.

### Step 2: N-(8-(methylamino)-5-(4-(trifluoromethoxy)phenethyl)-2,7-naphthyridin-3-yl)cyclopropanecarboxamide

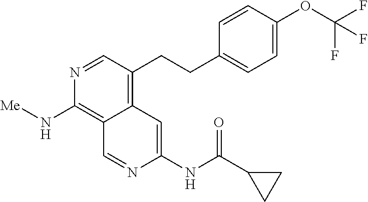 [SMILES: CNc1ncc(CCc2ccc(OC(F)(F)F)cc2)c2cc(NC(=O)C3CC3)ncc12]

A solution of (E)-N-(8-(methylamino)-5-(4-(trifluoromethoxy)styryl)-2,7-naphthyridin-3-yl) cyclopropanecarboxamide (20.0 mg; 0.047 mmol; 1.00 eq.) in :MeOH (15 mL) added 10% Pd/C (20 mg; 100% w/w) under nitrogen atmosphere. The mixture was hydrogenated at 40° C. for 3 hours under hydrogen atmosphere (2 atm). The desired product was detected via LCMS. After filtration, the filtrate was concentrated under reduced pressure. The crude product was purified by reverse phase preparative HPLC (Prep-C₁₋₈, 5 μM XBridge OBD column, 19×250 mm, waters; gradient elution of 47-57% MeCN in water over a 10 min period, where both water and MeCN contain 10 mmol/L NH₄HCO₃, flow rate: 60 mL/min, detector UV wavelength: 254 nm) to provide N-(8-(methylamino)-5-(4-(trifluoromethoxy)phenethyl)-2,7-naphthyridin-3-yl) cyclopropanecarboxamide as a white solid (3.5 mg, 17.4%). LCMS (ESI) m/z 431.2, [M+H]⁺.

### Example 849: Synthesis of N-(5-(4H-chromen-2-yl)-8-(methylamino)-2,7-naphthyridin-3-yl)cyclopropanecarboxamide

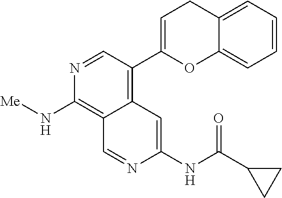 [SMILES: CNc1ncc(C2=CCc3ccccc3O2)c2cc(NC(=O)C3CC3)ncc12]

### Step 1: 4H-chromen-2-yl diphenyl phosphate

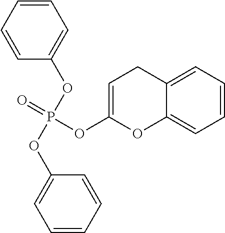 [SMILES: O=P(OC1=CCc2ccccc2O1)(Oc1ccccc1)Oc1ccccc1]

To a stirred solution of chroman-2-one (2.00 g; 13.49 mmol; 1.00 eq.) in THF (50.0 mL) was added LiHMDS (1 M in THF, 16.2 mL) dropwise at −78° C. under nitrogen atmosphere. The resulting solution was stirred at −78° C. for 15 minutes. To the above solution was diphenyl phosphorochloridate (4.35 g; 16.19 mmol; 1.20 eq.) at −78° C., the resulting mixture was allowed to warm to room temperature, and stirred for 30 minutes. The desired product was detected via LCMS. The reaction was quenched by adding saturated NaHCO₃ solution and extracted with EtOAc. The organic layer was washed with brine, dried over anhydrous Na₂SO₄, and concentrated under reduced pressure to afford 4H-chromen-2-yl diphenyl phosphate as a brown solid (1.5 g, crude). LCMS (ESI) m/z 381.1, [M+H]⁺.

### Step 2: N-(5-(4H-chromen-2-yl)-8-(methylamino)-2,7-naphthyridin-3-yl)cyclopropanecarboxamide

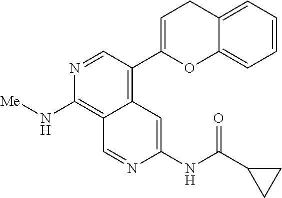 [SMILES: CNc1ncc(C2=CCc3ccccc3O2)c2cc(NC(=O)C3CC3)ncc12]

A solution of 4H-chromen-2-yl diphenyl phosphate (67.0 mg; 0.176 mmol; 1.00 eq.), Pd(t-Bu₃P)₂ (9.00 mg; 0.018 mmol; 0.01 eq.), K₃PO₄ (112.2 mg; 0.528 mmol; 3.00 eq.) and N-(8-(methylamino)-5-(4,4,5,5-tetramethyl-1,3,2-dioxaborolan-2-yl)-2,7-naphthyridin-3-yl)cyclopropanecarboxamide (36.0 mg; 0.098 mmol; 0.60 eq.) in a mixture solvent of MeCN/water (19:1, 1 mL) was stirred at 65° C. for 2 hours under nitrogen atmosphere. The desired product was detected via LCMS. The resulting mixture was concentrated under reduced pressure and purified by flash chromatography on silica gel column using 1-9% of MeOH in CH₂Cl₂ as eluent to provide a crude product. The product was purified by flash chromatography on pre-packed C18 column using 30-50% of MeCN in water (10 mmol/L NH₄HCO₃) to provide N-(5-(4H-chromen-2-yl)-8-(methylamino)-2,7-naphthyridin-3-yl)cyclopropanecarboxamide as an off-white solid (1.8 mg, 0.9%). LCMS (ESI) m/z 373.2, [M+H]⁺.

### Example 850: Synthesis of N-(5-(1H-isochromen-3-yl)-8-(methylamino)-2,7-naphthyridin-3-yl)cyclopropanecarboxamide

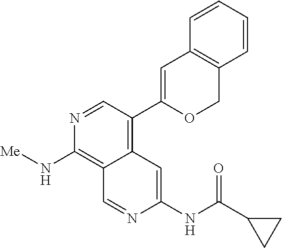 [SMILES: CNc1ncc(C2=Cc3ccccc3CO2)c2cc(NC(=O)C3CC3)ncc12]

### Step 1: N-(5-((2-formylphenyl)ethynyl)-8-(methylamino)-2,7-naphthyridin-3-yl)cyclopropanecarboxamide

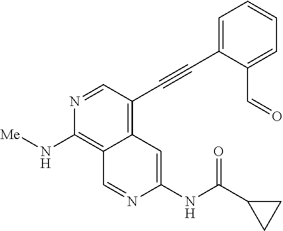 [SMILES: CNc1ncc(C#Cc2ccccc2C=O)c2cc(NC(=O)C3CC3)ncc12]

A solution of N-(5-bromo-8-(methylamino)-2,7-naphthyridin-3-yl)cyclopropanecarboxamide (140.0 mg; 0.436 mmol; 1.00 eq.), Pd(PPh₃)₂C1.2 (9.2 mg; 0.013 mmol; 0.03 eq.), CuI (1.7 mg; 0.009 mmol; 0.02 eq.) and diisopropylamine (132.3 mg; 1.308 mmol; 3.00 eq.) in a mixture solvent of toluene/DMSO (10:1, 4.4 mL) was stirred at 140° C. for 12 hours under nitrogen atmosphere. The desired product was detected via LCMS. The resulting mixture was concentrated under reduced pressure. The residue was purified by flash chromatography on silica gel column using 1-9% of MeOH in CH₂Cl₂ as eluent to provide N-(5-((2-formylphenyl)ethynyl)-8-(methylamino)-2,7-naphthyridin-3-yl)cyclopropanecarboxamide as a yellow solid (30.0 mg, 19.0%). LCMS (ESI) m/z 371.1, [M+H]⁺.

### Step 2: N-(5-(1H-isochromen-3-yl)-8-(methylamino)-2,7-naphthyridin-3-yl)cyclopropanecarboxamide

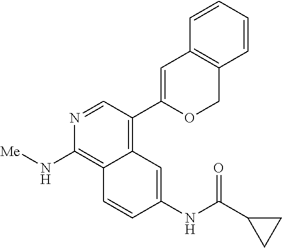 [SMILES: CNc1ncc(C2=Cc3ccccc3CO2)c2cc(NC(=O)C3CC3)ccc12]

A solution of N-(5-((2-formylphenyl)ethynyl)-8-(methylamino)-2,7-naphthyridin-3-yl)cyclopropanecarboxamide (25.0 mg; 0.067 mmol; 1.00 eq.), (pyridin-4-ylmethyl)gold(III) chloride (CAS: 88215-41-2) (1.2 mg; 0.003 mmol; 0.04 eq.) and diethyl 2,6-dimethyl-1,4-dihydropyridine-3,5-dicarboxylate (20.5 mg; 0.080 mmol; 1.20 eq.) in a mixture solvent of toluene/DMSO (1:5, 1.2 mL) was stirred at 80° C. for 12 hours under nitrogen atmosphere. The desired product was detected via LCMS. The resulting mixture was concentrated under reduced pressure and purified by flash chromatography on silica gel column using 1-9% of MeOH in CH₂Cl₂ as eluent to provide the crude product. The product was purified by flash chromatography on pre-packed C18 column using 20-50% of MeCN in water (10 mmol/L NH₄HCO₃) to provide N-(5-(1H-isochromen-3-yl)-8-(methylamino)-2,7-naphthyridin-3-yl)cyclopropanecarboxamide as a white solid (3.1 mg, 12.4%). LCMS (ESI) m/z 373.2, [M+H]⁺. ¹H NMR (400 MHz, DMSO-d₆) δ 11.00 (s, 1H), 9.35 (s, 1H), 8.69 (s, 1H), 8.22-8.16 (m, 1H), 8.15 (s, 1H), 7.32-7.08 (m, 4H), 6.23 (s, 1H), 5.24 (s, 2H), 3.01 (d, J=° 4.4 Hz, 3H), 2.13-2.02 (m, 1H), 0.91-0.78 (m, 4H).

### Example 851: Synthesis of N-(5-(4-(2-methyl-2/d-1,2,3-triazol-4-yl)phenyl)-8-(methylamino)-2,7-naphthyridin-3-yl)cyclopropanecarboxamide

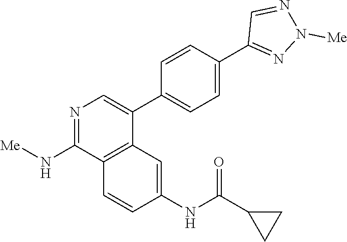 [SMILES: CNc1ncc(-c2ccc(-c3cnn(C)n3)cc2)c2cc(NC(=O)C3CC3)ccc12]

### Step 1: 4-(4-bromophenyl)-2-methyl-2H-1,2,3-triazole

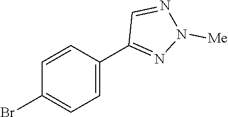 [SMILES: Cn1ncc(-c2ccc(Br)cc2)n1]

To a solution of 4-bromo-2-methyl-1,2,3-triazole (1.60 g; 9.877 mmol; 1.00 eq.) in a mixture solvent of DME/water (5:1, 12 mL) was added Pd(PPh₃)₄ (288.0 mg; 1.098 mmol; 0.11 eq.), Na₂CO₃ (530.0 mg; 5.001 mmol; 0.51 eq.) and (4-bromophenyl)boronic acid (500.0 mg; 2.490 mmol; 0.25 eq.). The reaction was stirred at 100° C. for 2 hours under nitrogen atmosphere. The desired product was detected via LCMS. The resulting mixture was diluted with EtOAc (20 mL) and washed by brine (3×20 mL). The organic layer was dried over anhydrous sodium sulfate and concentrated under vacuum. The residue was purified by flash chromatography on silica gel column using 20-70% of EtOAc in petroleum ether as eluent to provide 4-(4-bromophenyl)-2-methyl-21-1-1,2,3-triazole as a white oil (200.0 mg, 10.5%). LCMS (ESI) m/z 238.0, [M+H]⁺.

### Step 2: /V-(5-(4-(2-methyl-2H-1,2,3-triazol-4-yl)phenyl)-8-(methylamino)-2,7-naphthyridin-3-yl)cyclopropanecarboxamide

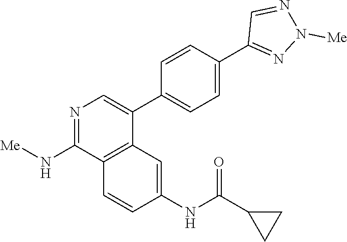 [SMILES: CNc1ncc(-c2ccc(-c3cnn(C)n3)cc2)c2cc(NC(=O)C3CC3)ccc12]

To a solution of 4-(4-bromophenyl)-2-methyl-2H-1,2,3-triazole (150.0 mg; 0.630 mmol; 1.00 eq.) in a mixture solvent of 1,4-dioxane/water (5:1, 6 mL) were added XPhos Pd G₃ (34.4 mg; 0.041 mmol; 0.10 eq.), XPhos (19.4 mg; 0.041 mmol; 0.10 eq.), K₃PO₄ (259.0 mg; 1.220 mmol; 3.00 eq.) and N-(8-(methylamino)-5-(4,4,5,5-tetramethyl-1,3,2-dioxaborolan-2-yl)-2,7-naphthyridin-3-yl) cyclopropanecarboxamide (232.0 mg; 0.630 mmol; 1.00 eq.). The reaction was stirred at 90° C. for 1 hour under nitrogen atmosphere. The desired product was detected via LCMS. The resulting mixture was concentrated under vacuum. The residue was purified by flash chromatography on silica gel column using 2-1.0% of MeOH in CH₂Cl₂ as eluent to provide 40 mg of the crude product. The product was re-purified by flash chromatography on pre-packed C18 column using 20-80% of MeCN in water (10 mmol/L NH₄HCO₃) as eluent to provide N-(5-(4-(2-methyl-2H-1,2,3-triazol-4-yl)phenyl)-8-(methylamino)-2,7-naphthyridin-3-yl)cyclopropanecarboxamide as a yellow solid (25.5 mg, 10.1%). LCMS (ESI) m/z 400.2, [M+H]⁺. ¹H NMR (400 MHz, DMSO-d₆) δ 10.96 (s, 1H), 9.40 (s, 1H), 8.41 (s, 1H), 8.30 (s, 1H), 8.06-8.02 (m, 1H), 7.99 (s, 1H), 7.94 (d, J=8.4 Hz, 2H), 7.51 (d, J=8.4 Hz, 2H), 4.23 (s, 3H), 3.03 (d, J=4.4 Hz, 3H), 2.07-1.97 (m, 1H), 0.81-0.75 (m, 4H).

### Example 852: Synthesis of N-(5-(1-(2-methoxyethyl)-1H-pyrazol-3-yl)-8-(methylamino)-2,7-naphthyridin-3-yl)cyclopropanecarboxamide

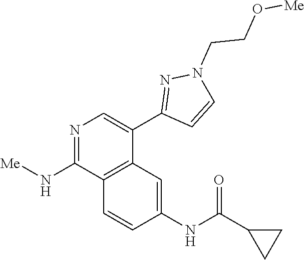 [SMILES: CNc1ncc(-c2ccn(CCOC)n2)c2cc(NC(=O)C3CC3)ccc12]

### Step 1: 3-bromo-1-(2-methoxyethyl)-1H-pyrazole

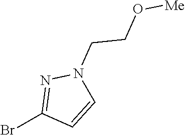

A solution of 3-bromo-1H-pyrazole (500.0 mg; 3.402 mmol; 1.00 eq.), 2-bromoethyl methyl ether (709.0 mg; 5.103 mmol; 1.50 eq.) and Cs₂CO₃ (2216.0 mg; 6.804 mmol; 2.00 eq.) in DMF (8 mL) was stirred at room temperature for 2 hours under nitrogen atmosphere. The desired product was detected via LCMS. The resulting mixture was diluted with water (20 mL) and extracted with EtOAc (2×30 mL). The combined organic layers were washed with brine (3×10 mL), dried over anhydrous Na₂SO₄. After filtration, the filtrate was concentrated under reduced pressure. The residue was purified by flash chromatography on silica gel column using 10-30% of EtOAc in petroleum ether as eluent to provide 3-bromo-1-(2-methoxyethyl)-1H-pyrazole as a yellow oil (140.0 mg, 21.0%). LCMS (ESI) m/z 205.0, [M+H]⁺.

### Step 2: V-(5-(1-(2-methoxyethyl)-.1H-pyrazol-3-yl)-8-(methylamino)-2,7-naphthyridin-3-yl)cyclopropanecarboxamide

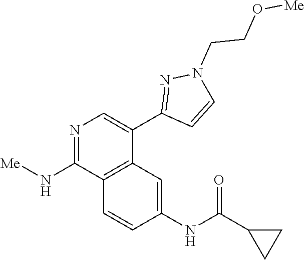 [SMILES: CNc1ncc(-c2ccn(CCOC)n2)c2cc(NC(=O)C3CC3)ccc12]

A solution of N-(8-(methylamino)-5-(4,4,5,5-tetramethyl-1,3,2-dioxaborolan-2-yl)-2,7-naphthyridin-3-yl)cyclopropanecarboxamide (140.0 mg; 0.380 mmol; 1.00 eq.), XPhos Pd G₃ (64.4 mg; 0.076 mmol; 0.02 eq.), XPhos (36.2 mg; 0.076 mmol; 0.02 eq.) and K₃PO:1 (242.1 mg; 1.140 mmol; 3.00 eq.) in a mixture solvent of 1,4-dioxane/water (10:1, 3.3 mL) was stirred at 90° C. for 1 hour under nitrogen atmosphere. The desired product was detected via LCMS. The resulting mixture was concentrated under reduced pressure and purified by flash chromatography on silica gel column using 1-9% of MeOH in CH₂Cl₂ as eluent to provide the crude product. The crude product was purified by flash chromatography on pre-packed C18 column using 10-70% of MeCN in water (10 mmol/L NH₄HCO₃) to provide N-(5-(1-(2-methoxyethyl)-1H pyrazol-3-yl)-8-(methylamino)-2,7-naphthyridin-3-yl)cyclopropanecarboxamide as a white solid (19.3 mg, 13.8%). LCMS (ESI) m/z 367.2, [M+H]⁺. ¹H NMR (400 MHz, DMSO-d₆) δ 10.89 (s, 1H), 9.35 (s, 1H), 9.08 (s, 1H), 8.21 (s, 1H), 8.00-7.92 (m, 1H), 7.79 (d, J=2.0 Hz, 1H), 6.48 (d, J=2.0 Hz, 1H), 4.30 (t, J=5.2 Hz, 2H), 3.82 (t, J=5.2 Hz, 2H), 3.27 (s, 3H), 3.01 (d, J=4.4 Hz, 3H), 2.11-2.00 (m, 1H), 0.87-0.76 (m, 4H).

### Example 853: Synthesis of N-(5-(6,7-dihydro-5H-pyrrolo[1,2-b][1,2,4]triazol-2-yl)-8-(methylamino)-2,7-naphthyridin-3-yl)cyclopropanecarboxamide formate

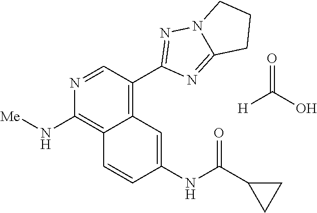

### Step 1: 6,7-dihydro-5H-pyrrolo[1,2-b][1,2,4]triazol-2-amine

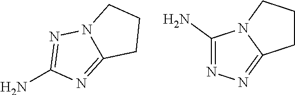

A solution of hydrazinecarboximidamide carbonate (4.00 g; 29.387 mmol; 4.01 eq.) and methyl 4-chlorobutanoate (1.00 g; 7.321 mmol; 1.00 eq.) in ethane-1,2-diol (20 mL) was stirred at 110° C. for 16 hours. The desired product was detected via LCMS. The resulting mixture was used in the next step directly without further purification. LCMS (ESI) m/z 125.1, [M+H]⁺.

### Step 2: 2-chloro-6,7-dihydro-5H-pyrrolo[1,2-b][1,2,4]triazole

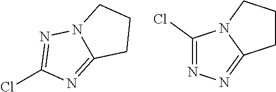

To a solution of 6,7-dihydro-5H-pyrrolo[1,2-b][1,2,4]triazol-2-amine (crude, estimated −20% content) in HCl (12 M, 60 mL) was added CuCl₂ (1.34 g; 9.967 mmol; 9.98 eq.) under nitrogen atmosphere. The mixture was allowed to cool down to 0° C. To the above solution was added dropwise a solution of NaNO₂ (690.0 mg; 10.001 mmol; 10.01 eq.) in water (20 mL). The resulting solution was stirred at room temperature for 1 hour. The desired product was detected via LCMS. The mixture was concentrated under reduced pressure and purified by flash chromatography on pre-packed C18 column using 20-80% of MeCN in water (10 mmol/L NH₄HCO₃) to afford 2-chloro-6,7-dihydro-5H-pyrrolo[1,2-b][1,2,4]triazole as a brown solid (80 mg, 8.8%) and 3-chloro-6,7-dihydro-5H-pyrrolo[2,1-c][1,2,4]triazole as a brown solid (71 mg, 8.0%). LCMS (ESI) m/z 144.0, [M+H]⁺.

### Step 3: N-(5-(6,7-dihydro-5H-pyrrolo[1,2-b][1,2,4]triazol-2-yl)-8-(methylamino)-2,7-naphthyridin-3-yl)cyclopropanecarboxamide formate

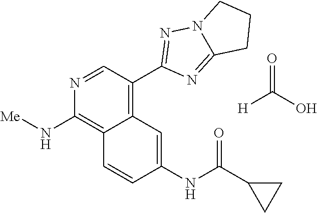

To a solution of 2-chloro-6,7-dihydro-5H-pyrrolo[1,2-b][1,2,4]triazole (71.0 mg; 0.495 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (182.0 mg; 0.494 mmol; 1.00 eq.) in a mixture solvent of 1,4-dioxane/water (5:1, 1.2 mL) were added XPhos Pd G₃ (40.0 mg; 0.047 mmol; 0.10 eq.), XPhos (46.0 mg; 0.096 mmol; 0.20 eq.) and K₃PO₄ (212.0 mg; 0.999 mmol; 2.02 eq.) under nitrogen atmosphere. The resulting mixture was stirred at 90° C. for 1 hour. The desired product was detected via LCMS. The resulting mixture was concentrated under reduced pressure and purified by flash chromatography on silica gel column using 5-10% of MeOH in CH₂Cl₂ as eluent to afford the crude product. The crude product was repurified by flash chromatography on pre-packed C₁₋₈ column using 40-80% of MeCN in water (10 mmol/L NH₄HCO₃) to afford N-(5-(6,7-dihydro-5H-pyrrolo[1,2-b][1,2,4]triazol-2-yl)-8-(methylamino)-2,7-naphthyridin-3-yl)cyclopropanecarboxamide formate as a off-white solid (6.3 mg, 3.2%). LCMS (ESI) m/z 350.2, [M+H]⁺.

### Example 854: Synthesis of N-(5-(6,7-dihydro-5H-pyrrolo[2,1-c][1,2,4]triazol-3-yl)-8-(methylamino)-2,7-naphthyridin-3-yl)cyclopropanecarboxamide

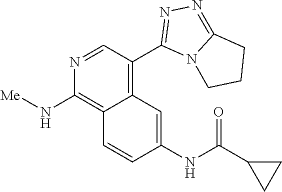 [SMILES: CNc1ncc(-c2nnc3n2CCC3)c2cc(NC(=O)C3CC3)ccc12]

To a solution of 3-chloro-6,7-dihydro-5H-pyrrolo[2,1-c][1,2,4]triazole (Example 853, step 2) (71.0 mg; 0.495 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (182.0 mg; 0.494 mmol; 1.00 eq.) in a mixture solvent of 1,4-dioxane/water (5:1, 1.2 mL) were added XPhos Pd G₃ (40.0 mg; 0.047 mmol; 0.10 eq.), XPhos (46.0 mg; 0.096 mmol; 0.20 eq.) and K₃PO₄ (212.0 mg; 0.999 mmol; 2.02 eq.) under nitrogen atmosphere. The resulting mixture was stirred at 90° C. for 1 hour. The desired product was detected via LCMS. The resulting mixture was concentrated under reduced pressure. The residue was purified by flash chromatography on silica gel column using 5-10% of MeOH in CH₂Cl₂ as eluent to afford the crude product. The crude product was purified by flash chromatography on pre-packed C18 column using 10-50% of MeCN in water (10 mmol/L NH₄HCO₃) to afford N-(5-(6,7-dihydro-5H-pyrrolo[2,1-c][1,2,4]triazol-3-yl)-8-(methylamino)-2,7-naphthyridin-3-yl)cyclopropanecarboxamide as an off-white solid (3.3 mg, 1.9%). LCMS (ESI) m/z 350.2, [M+H]⁺.

### Example 855: Synthesis of N-(8-(methylamino)-5-(5-(trifluoromethyl)-4,5,6,7-tetrahydrobenzo[d]oxazol-2-yl)-2,7-naphthyridin-3-yl)cyclopropanecarboxamide

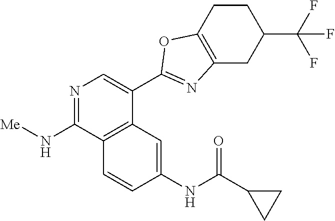 [SMILES: CNc1ncc(-c2nc3c(o2)CCC(C(F)(F)F)C3)c2cc(NC(=O)C3CC3)ccc12]

### Step 1: 2-bromo-4-(trifluoromethyl)cyclohexan-1-one

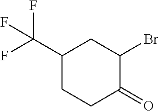 [SMILES: O=C1CCC(C(F)(F)F)CC1Br]

To a solution of 4-(trifluoromethyl)cyclohexan-1-one (2.00 g; 12.040 mmol; 1.00 eq.) in diethyl ether (40 mL) was added bromine (1.90 g; 12.040 mmol; 1.00 eq.) at 0° C. The resulting mixture was stirred at 0° C. for 1 h under nitrogen atmosphere. The desired product was detected via TLC. After the reaction was completed, the mixture was quenched with sodium thiosulfate solution and diluted with ice water. (50 mL). The resulting mixture was extracted with ethyl acetate (3×30 mL). The combined organic layer was washed with brine, dried over anhydrous sodium sulfate and filtered. The filtrate was concentrated under vacuum to afford 2-bromo-4-(trifluoromethyl)cyclohexan-1-one as a yellow oil (2.9 g, crude).

### Step 2: 5-(trifluoromethyl)-4,5,6,7-tetrahydrobenzo[d]oxazol-2-amine

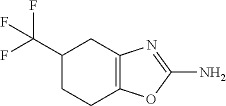 [SMILES: Nc1nc2c(o1)CCC(C(F)(F)F)C2]

To a solution of 2-bromo-4(trifluoromethyl)cyclohexan-1-one (1.00 g; 4.080 mmol; 1.00 eq.) in EtOH (20 mL) was added urea (735.3 mg; 1.2.240 mmol; 3.00 eq.). The resulting mixture was stirred at 80° C. for 16 hours under nitrogen atmosphere. The desired product was detected via LCMS. After the reaction was completed, the mixture was concentrated under vacuum and purified by flash chromatography on silica gel column using 1-9% of MeOH in CH₂Cl₂ as eluent to provide 5-(trifluoromethyl)-4,5,6,7-tetrahydrobenzo[d]oxazol-2-amine as a yellow solid (306.0 mg, 36.2%). LCMS (ESI) m/z 207.1, [M+H]⁺.

### Step 3: 2-iodo-5-(trifluoromethyl)-4,5,6,7-tetrahydrobenzo[d]oxazole

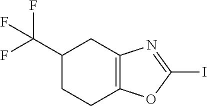 [SMILES: FC(F)(F)C1CCc2oc(I)nc2C1]

To a solution of 5-(trifluoromethyl)-4,5,6,7-tetrahydrobenzo[d]oxazol-2-amine (246.0 mg; 1.190 mmol; 1.00 eq.) in MeCN (10 mL) were added CuI (454.5 mg; 2.390 mmol; 2.00 eq.) and isoamyl nitrite (279.6 mg; 2.39 mmol; 2.00 eq.). The resulting mixture was stirred at room temperature for 5 hours. The desired product was detected via TLC. After the reaction was completed, the mixture was diluted with water (10 mL). To the mixture was added NH₃·H₂O until the mixture was clear. The resulting mixture was extracted with ethyl acetate (3×20 mL). The combined organic layer was washed with brine, dried over anhydrous sodium sulfate and filtered. The filtrate was concentrated under vacuum and purified by flash chromatography on silica gel column using 1.0-75% of ethyl acetate in petroleum ether as eluent to provide 2-iodo-5-(trifluoromethyl)-4,5,6,7-tetrahydrobenzo[d]oxazole as a yellow green solid (25.0 mg, 6.6%).

### Step 4: N-(8-(methylamino)-5-(5-(trifluoromethyl)-4,5,6,7-tetrahydrobenzo[d]oxazol-2-yl)-2,7-naphthyridin-3-yl)cyclopropanecarboxamide

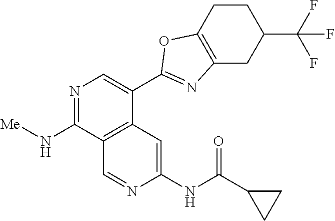 [SMILES: CNc1ncc(-c2nc3c(o2)CCC(C(F)(F)F)C3)c2cc(NC(=O)C3CC3)ncc12]

To a solution of N-(8-(methylamino)-5-(4,4,5,5-tetramethyl-1,3,2-dioxaborolan-2-yl)-2,7-naphthyridin-3-yl)cyclopropanecarboxamide (29.0 mg; 0.078 mmol; 1.00 eq.), 2-iodo-5-(trifluoromethyl)-4,5,6,7-tetrahydrobenzo[d]oxazole (25.0 mg; 0.080 mmol; 1.00 eq.) in a mixture solvent of 1,4-dioxane/water (5:1, 1.2 mL) were added XPhos Pd G₃ (6.7 mg; 0.010 mmol; 0.12 eq.), XPhos (3.8 mg; 0.01 mmol; 0.12 eq.) and K₃PO₄ (50.2 mg; 0.240 mmol; 3.00 eq.). The resulting mixture was stirred at 60° C. for 1 hour under nitrogen atmosphere. The desired product was detected via LCMS. After the reaction was completed, the mixture was concentrated under vacuum and purified by flash chromatography on silica gel column using 10-75% of ethyl acetate in petroleum ether as eluent to provide the crude product. The product was purified by reverse phase preparative HPLC (Prep-C18, 5 μM XBridge OBD column, 19×250 mm, waters; gradient elution of 43-53% MeCN in water over a 10 min period, where both water and MeCN contain 10 mmol/L NH₄HCO₃, flow rate: 60 mL/min, detector UV wavelength: 254 nm) to afford N-(8-(methylamino)-5-(5-(trifluoromethyl)-4,5,6,7-tetrahydrobenzo[d]oxazol-2-yl)-2,7-naphthyridin-3-yl)cyclopropanecarboxamide as a light yellow solid (5.3 mg, 15.6%). LCMS (ESI) m/z 432.2, [M+H]⁺. H NMR (400 MHz, DMSO-d₆) δ 10.95 (s, 1H), 9.40 (s, 1H), 9.39 (s, 1H), 8.61 (s, 1H), 8.44-8.35 (m, 1H), 3.04 (d, J=4.4 Hz, 3H), 3.01-2.98 (m, 2H), 2.85-2.74 (m, 1H), 2.72-2.61 (m, 2H), 2.20-2.02 (m, 2H), 1.85-1.72 (m, 1H), 0.90-0.81 (m, 41-1).

### Example 856: Synthesis of N-(5-(2-bromo-[1,2,4]triazolo[1,5-a]pyridin-8-yl)-8-(methylamino)-2,7-naphthyridin-3-yl)cyclopropanecarboxamide

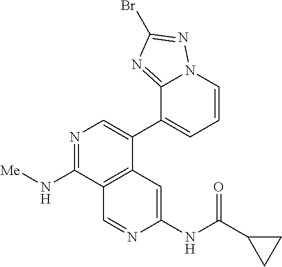 [SMILES: CNc1ncc(-c2cccn3nc(Br)nc23)c2cc(NC(=O)C3CC3)ncc12]

### Step 1: 2-bromo-8-chloro-[1,2,4]triazolo[1,5-a]pyridine

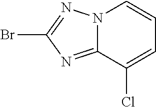 [SMILES: Clc1cccn2nc(Br)nc12]

A solution of 8-chloro-[1,2,4]triazolo[1,5-a]pyridin-2-amine (200.0 mg; 1.186 mmol; 1.00 eq.), tert-butyl nitrite (244.7 mg; 2.372 mmol; 2.00 eq.) and CuBr (340.4 mg; 2.372 mmol; 2.00 eq.) in MeCN (5 mL) was stirred at 60° C. for 1 hour under nitrogen atmosphere. The desired product was detected via LCMS. The residue was acidified to pH=2 with 1 M HCl. The resulting mixture was extracted with EtOAc (2×20 mL). The combined organic layers were washed with brine (3×4 mL), dried over anhydrous Na₂SO₄. After filtration, the filtrate was concentrated under reduced pressure to afford 2-bromo-8-chloro-[1,2,4]triazolo[1,5-a]pyridine as a yellow solid (178.0 mg, 65%). LCMS (ESI) m/z 231.9, [M+H]⁺.

### Step 2: N-(5-(2-bromo-[1,2,4]triazolo[1,5-a]pyridin-8-yl)-8-(methylamino)-2,7-naphthyridin-3-yl)cyclopropanecarboxamide

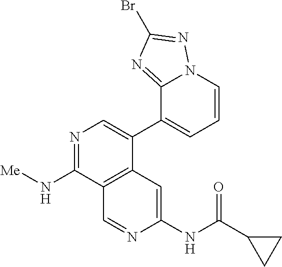 [SMILES: CNc1ncc(-c2cccn3nc(Br)nc23)c2cc(NC(=O)C3CC3)ncc12]

A solution of N-(8-(methylamino)-5-(4,4,5,5-tetramethyl-1,3,2-dioxaborolan-2-yl)-2,7-naphthyridin-3-yl)cyclopropanecarboxamide (50.0 mg; 0.136 mmol; 1.00 eq.), 2-bromo-8-chloro-[1,2,4]triazolo[1,5-a]pyridine (94.7 mg; 0.408 mmol; 3.00 eq.), XPhos Pd G₃ (23.0 mg; 0.027 mmol; 0.20 eq.), XPhos (12.9 mg; 0.027 mmol; 0.20 eq.) and K₃PO₄ (86.5 mg; 0.408 mmol; 3.00 eq.) in a mixture solvent of 1,4-dioxane/water (10:1, 1.1 mL) was stirred at 90° C. for 1 hour under nitrogen atmosphere. The desired product was detected via LCMS. The resulting mixture was concentrated under reduced pressure. The residue was purified by flash chromatography on silica gel column using 1-9% of MeOH in CH₂Cl₂ as eluent to provide the crude product that was repurified by flash chromatography on pre-packed C18 column using 10-70% of MeCN in water (10 mmo/L NH₄HCO₃) as eluent to provide N-(5-(2-bromo-[1,2,4]triazolo[1, 5-a]pyridin-8-yl)-8-(methylamino)-2,7-naphthyridin-3-yl)cyclopropanecarboxamide as a brown solid (3.7 mg, 6.2%). LCMS (ESI) m/z 438.1, [M+H]⁺.

### Example 857: Synthesis of N-(8-(methylamino)-5-(4-(thiazol-5-yloxy)phenyl)-2,7-naphthyridin-3-yl)cyclopropanecarboxamide

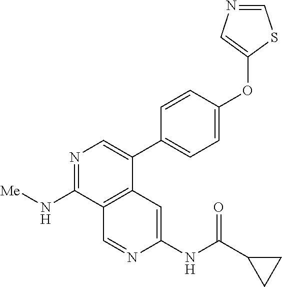 [SMILES: CNc1ncc(-c2ccc(Oc3cncs3)cc2)c2cc(NC(=O)C3CC3)ncc12]

### Step 1: 5-(4-bromophenoxy)thiazole

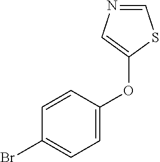 [SMILES: Brc1ccc(Oc2cncs2)cc1]

A solution of 4-bromophenol (1.32 g; 7.621 mmol; 1.25 eq.), Cs₂CO₃ (5.96 g; 18.292 mmol; 3.00 eq.), CuI (1.16 g; 6.091 mmol; 1.00 eq.) and 5-bromothiazole (1.00 g; 6.097 mmol; 1.00 eq.) in DMF (24 mL) was stirred at 110° C. for 5 hours under nitrogen atmosphere. The desired product was detected via LCMS. The resulting mixture was diluted with EtOAc (200 mL), washed with brine (3<20 mL), dried over anhydrous Na₂SO₄. After filtration, the filtrate was concentrated under reduced pressure. The residue was purified by flash chromatography on silica gel column using 10-20% of EtOAc in petroleum ether as eluent to provide 5-(4-bromophenoxy)thiazole as a brown solid (390 mg, 24.9%). LCMS (ESI) m/z 255.9, [M+H]⁺.

### Step 2: N-(8-(methylamino)-5-(4-(thiazol-5-yloxy)phenyl)-2,7-naphthyridin-3-yl)cyclopropanecarboxamide

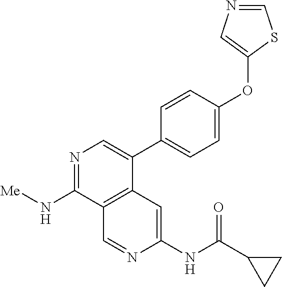 [SMILES: CNc1ncc(-c2ccc(Oc3cncs3)cc2)c2cc(NC(=O)C3CC3)ncc12]

A solution of 5-(4-bromophenoxy)thiazole (69.6 mg; 0.272 mmol; 1.00 eq.), K₃PO₄. (172.9 mg; 0.815 mmol; 3.00 eq.), XPhos (12.9 mg; 0.027 mmol; 0.10 eq.), XPhos Pd G₃ (23.0 mg; 0.027 mmol; 0.10 eq.) and N-(0,8-(methylamino)-5-(4,4,5,5-tetramethyl-1,3,2-dioxaborolan-2-yl)-2,7-naphthyridin-3-yl)cyclopropanecarboxamide (102 mg; 0.274 mmol; 1.00 eq.) in a mixture solvent of 1,4-dioxane/water (5:1, 1.2 mL) was stirred at 90° C. for 1 hour under nitrogen atmosphere. The desired product was detected via LCMS. The resulting mixture was concentrated under reduced pressure. The residue was purified by flash chromatography on silica gel column using 1-9% of MeOH in CH₂C17 as eluent to provide a crude product that was purified by flash chromatography on pre-packed C18 column using 10-50% of MeCN in water (10 mmol/L NH₄HCO₃) to provide N-(8-(methylamino)-5-(4-(thiazol-5-yloxy)phenyl)-2,7-naphthyridin-3-yl)cyclopropanecarboxamide as a white solid (17.4 mg, 15.3%). LCMS (ESI) m/z 418.1, [M+H]⁺. ¹H NMR (400 MHz, DMSO-d₆) δ 10.94 (s, 1H), 9.38 (s, 1H), 8.82 (s, 1H), 8.31 (s, 1H), 8.02-7.94 (m, 141, 7.92 (s, 14), 7.72 (s, 14), 7.44 (d, J=8.4 Hz 2H), 7.25 (d, J=8.4 Hz, 2H), 3.01 (d, J=4.4 Hz, 3H), 2.08-1.97 (m, 1H), 0.82-0.75 (m, 4H).

### Example 858: Synthesis of N-(8-(methylamino)-5-(4-(pyridin-4-yloxy)phenyl)-2,7-naphthyridin-3-yl)cyclopropanecarboxamide

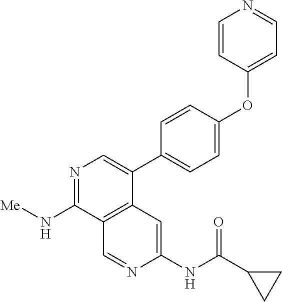 [SMILES: CNc1ncc(-c2ccc(Oc3ccncc3)cc2)c2cc(NC(=O)C3CC3)ncc12]

To a solution of N-(8-(methylamino)-5-(4,4,5,5-tetramethyl-1,3,2-dioxaborolan-2-yl)-2,7-naphthyridin-3-yl)cyclopropanecarboxamide (150.0 mg; 0.407 mmol; 1.00 eq.) and 4-(4-bromophenoxy)pyridine (101.8 mg; 0.407 mmol; 1.00 eq.) in a mixture solvent of 1,4-dioxane/water (10:1, 4.4 mL) were added XPhos Pd B₃ (34.4 mg; 0.041 mmol; 0.10 eq.), XPhos (19.4 mg; 0.041 mmol; 0.10 eq.) and K₃PO₄ (262.8 mg; 1.237 mmol; 3.04 eq.). The resulting solution was stirred at 90° C. for 1 hour under nitrogen atmosphere. The desired product was detected via LCMS. The mixture was allowed to cool down to room temperature. The resulting mixture was concentrated under reduced pressure. The residue was purified by flash chromatography on silica gel column using 10-20% of MeOH in CH₂Cl₂ as eluent to provide the crude product. The crude product was purified by flash chromatography on pre-packed C18 column using 50-90% of MeOH in water (10 mmol/L NH₄HCO₃) as eluent to provide N-(8-(methylamino)-5-(4-(pyridin-4-yloxy)phenyl)-2,7-naphthyridin-3-yl)cyclopropanecarboxamide as a white solid (48.2 mg, 28.7%). LCMS (ESI) m/z 412.2, [M+H]⁺. ¹H NMR (400 MHz, DMSO-d₆) δ 10.96 (s, 1H), 9.39 (s, 1H), 8.56-8.48 (m, 2H), 8.34 (s, 1H), 8.06-7.96 (m, 2H), 7.56-7.49 (m, 2H), 7.35-7.29 (m, 2H), 7.05-6.99 (m, 2H), 3.02 (d, J=4.4 Hz, 3H), 2.07-1.98 (m, 1H), 0.84-0.75 (m, 4H).

### Example 859: Synthesis of N-(5-(4-(2-methoxyethoxy)phenyl)-8-(methylamino)-2,7-naphthyridin-3-yl)cyclopropanecarboxamide

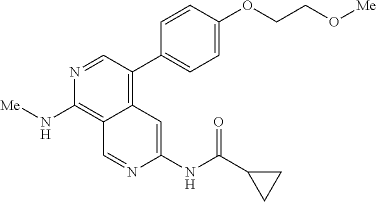 [SMILES: CNc1ncc(-c2ccc(OCCOC)cc2)c2cc(NC(=O)C3CC3)ncc12]

### Step 1: 1-bromo-4-(2-methoxyethoxy)benzene

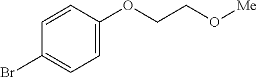 [SMILES: COCCOc1ccc(Br)cc1]

To a solution of 4-bromophenol (500.0 mg; 2.890 mmol; 1.00 eq.) and K₂CO₃ (1.1.9 g; 8.670 mmol; 3.00 eq.) in DMF (5 mL) was added 1-bromo-2-methoxyethane (803.4 mg; 5.780 mmol; 2.00 eq.). The mixture was stirred at 60° C. for 16 hours under nitrogen atmosphere. The resulting mixture was purified by flash chromatography on pre-packed C₁₋₈ column using 30-100% of MeCN in water (10 mmol/L NH₄HCO₃) as eluent to provide 1-bromo-4-(2-methoxyethoxy)benzene as a yellow solid (574.0 mg, 86.0%). ¹H: NMR (400 MHz, DMSO-d₆) δ 7.52-7.34 (m, 2H), 6.99-6.87 (m, 2H), 4.15-4.02 (m, 2H), 3.72-3.60 (m, 2H), 3.32 (s, 3H).

### Step 2: N-(5-(4-(2-methoxyethoxy)phenyl)-8-(methylamino)-2,7-naphthyridin-3-yl)cyclopropanecarboxamide

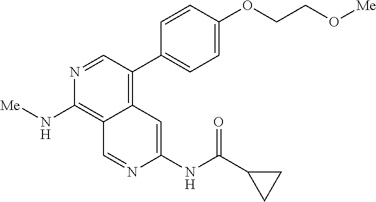 [SMILES: CNc1ncc(-c2ccc(OCCOC)cc2)c2cc(NC(=O)C3CC3)ncc12]

A solution of 1-bromo-4-(2-methoxyethoxy)benzene (31.4 mg; 0.136 mmol; 1.00 eq.), N-(8-(methylamino)-5-(4,4,5,5-tetramethyl-1,3,2-dioxaborolan-2-yl)-2,7-naphthyridin-3-yl)cyclopropanecarboxamide (50.0 mg; 0.1.36 mmol; 1.00 eq.), XPhos Pd G₃ (11.5 mg; 0.01.4 mmol; 0.10 eq.), XPhos (6.5 mg; 0.014 mmol; 0.10 eq.), K₃PO₄ (86.5 mg; 0.408 mmol; 3.00 eq.) in a mixture solvent of 1,4-dioxane/water. (5:1, 1.8 mL) was stirred at 90° C. for 1 hour under nitrogen atmosphere. The resulting mixture was concentrated under reduced pressure and purified by flash chromatography on silica gel column using 0-25% of MeOH in CH₂Cl₂ as eluent to provide the crude product. The crude product was purified by flash chromatography on pre-packed C18 column using 20-100% of MeCN in water (10 mmol/L NH₄HCO₃) as eluent to provide N-(5-(4-(2-methoxyethoxy)phenyl)-8-(methylamino)-2,7-naphthyridin-3-yl)cyclopropanecarboxamide as a yellow solid (1.2.4 mg, 23.3%). LCMS (ESI) m/z 393.2, [M+H]⁺. ¹H NMR (400 MHz, DMSO-d₆) δ 11.01 (s, 1H), 9.41 (s, 1H), 8.32 (s, 1H), 8.20-8.17 (m, 1H), 7.83 (s, 1H), 7.31 (d, J=8.4 Hz, 2H), 7.06 (d, J=8.4 Hz, 2H), 4.16 (t, 0.1=4.4 Hz, 2H), 3.70 (t, J=4.4 Hz, 2H), 3.34 (s, 3H), 3.03 (d, J=4.4 Hz, 3H), 2.09-1.99 (m, 1H), 0.84-0.75 (m, 4H).

### Example 860: Synthesis of N-(5-(4-(2-(cyanomethyl)azetidin-1-yl)phenyl)-8-(methylamino)-2,7-naphthyridin-3-yl)cyclopropanecarboxamide

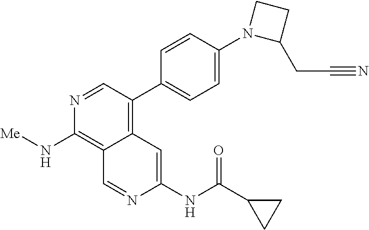 [SMILES: CNc1ncc(-c2ccc(N3CCC3CC#N)cc2)c2cc(NC(=O)C3CC3)ncc12]

### Step 1: azetidin-2-ylmethanol hydrochloride

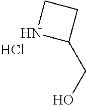

To a stirred solution of tert-butyl 2-(hydroxymethyl)azetidine-1-carboxylate (2.00 g; 10.6 mmol; 1.00 eq.) in MeOH (10 mL) was added TCl (4 M in 1,4-dioxane, 25 mL) dropwise at 0° C. The resulting mixture was stirred at room temperature for 1 hour under nitrogen atmosphere. The mixture was concentrated under vacuum to afford azetidin-2-ylmethanol hydrochloride. The 1.5 crude product was used for the next step directly without further purification.

### Step 2: (1-(4-bromophenyl)azetidin-2-yl)methanol

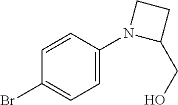 [SMILES: OCC1CCN1c1ccc(Br)cc1]

A solution of azetidin-2-ylmethanol hydrochloride (300.0 mg; 2.420 mmol; 1.00 eq.), CuI (1.1.1 mg; 0.058 mmol; 0.02 eq.), NaOH: (284.1 mg; 7.260 mmol; 3.00 eq.) and 1-bromo-4-iodobenzene (727.1 mg; 2.662 mmol; 1.10 eq.) in i-PrOH (3 mL) was stirred at 95° C. for 3 hours under nitrogen atmosphere. The desired product was detected via LCMS. The resulting mixture was diluted with EtOAc (80 mL), washed with brine (2×15 mL), dried over anhydrous Na₂SO₄. After filtration, the filtrate was concentrated under reduced pressure. The residue was purified by flash chromatography on silica gel column using 0-30% of EtOAc in petroleum ether as eluent to provide (1-(4-bromophenyl)azetidin-2-yl)methanol as a brown oil (341.1 mg, 58.0%). LCMS (ESI) m/z 242.0, [M+H]⁺.

### Step 3: (1-(4-bromophenyl)azetidin-2-yl)methyl methanesulfonate

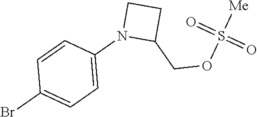 [SMILES: CS(=O)(=O)OCC1CCN1c1ccc(Br)cc1]

To a stirred solution of (1-(4-bromophenyl)azetidin-2-yl)methanol (441.1 mg; 1.820 mmol; 1.00 eq.) and DIPEA (368.0 mg; 3.64 mmol; 2.00 eq.) in CH₂Cl₂ (5 mL) was added methanesulfonic anhydride (475.1 mg; 2.730 mmol; 1.50 eq.) dropwise at 0° C. The resulting mixture was stirred for 1 h at room temperature under nitrogen atmosphere. The reaction was quenched by the 1.5 addition of water (1 mL) at room temperature. The resulting mixture was diluted with CH₂Cl₂ (80 mL), washed with brine (2×10 mL), dried over anhydrous Na₂SO₄. After filtration, the filtrate was concentrated under reduced pressure to afford (1-(4-bromophenyl)azetidin-2-yl)methyl methanesulfonate as a brown solid (300 mg, crude). LCMS (ESI) m/z 320.0, [M+H]⁺.

### Step 4: 2-(1-(4-bromophenyl)azetidin-2-yl)acetonitrile

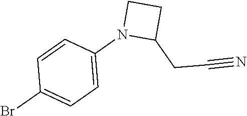 [SMILES: N#CCC1CCN1c1ccc(Br)cc1]

To a stirred solution of (1-(4-bromophenyl)azetidin-2-yl)methyl methanesulfonate (300.0 mg; 0.937 mmol; 1.00 eq.) in MeCN (3 mL) was added trimethylsilanecarbonitrile (185.0 mg; 1.874 mmol; 2.00 eq.) and TBAF (1.86 mL; 1.865 mmol; 1.99 eq.) dropwise at 0° C. The resulting mixture was stirred overnight at room temperature under nitrogen atmosphere. The reaction was quenched by the addition of water (3 mL) at room temperature. The resulting mixture was diluted with EtOAc (60 mL), washed with brine (2×10 mL), dried over anhydrous Na₂SO₄. After filtration, the filtrate was concentrated under reduced pressure. The residue was purified by flash chromatography on silica gel column using 0-50% of EtOAc in petroleum ether as eluent to provide 2-(1-(4-bromophenyl)azetidin-2-yl)acetonitrile as an off-white solid (81.0 mg, 34.4%). LCMS (ESI) m/z 251.0, [M+H]⁺.

### Step 5: N-(5-(4-(2-(cyanomethyl)azetidin-1-yl)phenyl)-8-(methylamino)-2,7-naphthyridin-3-yl)cyclopropanecarboxamide

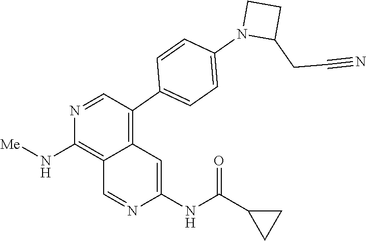 [SMILES: CNc1ncc(-c2ccc(N3CCC3CC#N)cc2)c2cc(NC(=O)C3CC3)ncc12]

A solution of N-(8-(methylamino)-5-(4,4,5,5-tetramethyl-1,3,2-dioxaborolan-2-yl)-2,7-naphthyridin-3-yl)cyclopropanecarboxamide (58.6 mg; 0.159 mmol; 1.00 eq.), 2-(1-(4-bromophenyl)azetidin-2-yl)acetonitrile (40 mg; 0.159 mmol; 1.00 eq.), XPhos Pd G₃ (13.5 mg; 0.016 mmol; 0.10 eq.), XPhos (1.5.2 mg; 0.032 mmol; 0.20 eq.) and K₃PO₄ (67.6 mg; 0.318 mmol; 2.00 eq.) in a mixture solvent of 1,4-dioxane/water (5:1, 1.2 mL) was stirred at 90° C. for 1 hour under nitrogen atmosphere. The desired product was detected via LCMS. The resulting mixture was concentrated under reduced pressure and purified by flash chromatography on silica gel column using 3-10% of MeOH in CH₂Cl₂ as eluent to provide the crude product. The product was purified by flash chromatography on pre-packed C18 column using 20-100% of MeCN in water (10 mmol/L NH₄HCO₃) as eluent to provide N-(5-(4-(2-(cyanomethyl)azetidin-1-yl)phenyl)-8-(methylamino)-2,7-naphthyridin-3-yl)cyclopropanecarboxamide as a yellow solid (22.7 mg, 34.5%). LCMS (ESI) m/z 413.2, [M+H]⁺. ¹H NMR (400 MHz, DMSO-d₆) δ 10.90 (s, 1H), 9.36 (s, 1H), 8.34 (s, 1H), 7.91-7.86 (m, 1H), 7.85 (s, 1H), 7.21 (d, J=° 8.4 Hz, 2H), 6.66 (d, J=8.4 Hz, 2H), 4.37-4.26 (m, 1H), 4.02-3.92 (m, 1H), 3.70-3.59 (m, 1H), 3.21-3.03 (m, 2H), 3.00 (d, J=4.4 Hz, 3H), 2.55-2.50 (m, 1H), 2.38-2.24 (m, 1H), 2.09-1.96 (m, 1H), 0.87-0.72 (m, 4H).

### Example 861: Synthesis of N-(5-(4-hydroxyphenyl)-8-(methylamino)-2,7-naphthyridin-3-yl)cyclopropanecarboxamide

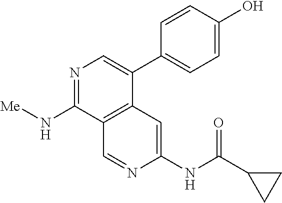 [SMILES: CNc1ncc(-c2ccc(O)cc2)c2cc(NC(=O)C3CC3)ncc12]

A solution of N-(8-(methylamino)-5-(4,4,5,5-tetramethyl-1,3,2-dioxaborolan-2-yl)-2,7-naphthyridin-3-yl)cyclopropanecarboxamide (200.0 mg; 0.543 mmol; 1.00 eq.), 4-bromophenol (94.0 mg; 0.370 mmol; 1.00 eq.), XPhos Pd C:3 (46.0 mg; 0.054 mmol; 0.10 eq.), XPhos (25.9 mg; 0.054 mmol; 0.10 eq.) and K₃PO₄ (345.6 mg; 1.628 mmol; 3.00 eq.) in a mixture solvent of 1,4-dioxane/water (5:1, 3 mL) was stirred at 90° C. for 1 hour under nitrogen atmosphere. The reaction was concentrated under pressure and purified by flash chromatography on silica gel column using 0-25% of MeOH in CH₂Cl₂ as eluent to provide the crude product. The crude product was repurified by reverse phase preparative HPLC (Prep-C18, 5 μM XBridge OBD column, 30×150 mm, water; gradient elution of 20-30% MeCN in water over a 12 min period, where water contains 10 mmol/L NH₄HCO₃, flow rate: 60 mL/min, detector UV wavelength: 254 nm) to give N-(5-(4-hydroxyphenyl)-8-(methylamino)-2,7-naphthyridin-3-yl)cyclopropanecarboxamide as a light yellow solid (16.2 mg, 8.9%). LCMS (ESI) m/z 334.1, [M+H]⁺. ¹H NMR (400 MHz, DMSO-d₆) δ 10.91 (s, 1H), 9.50 (s, 1H), 9.35 (s, 1H), 8.32 (s, 1H), 7.91-7.85 (m, 1H), 7.86 (s, 1H), 7.18 (d, J=8.4 Hz, 2H), 6.86 (d, J=8.4 Hz, 2H), 2.99 (d, J=4.4 Hz, 3H), 2.08-1.92 (m, 1H), 0.89-0.74 (m, 4H).

### Example 862: Synthesis of N-(8-(methylamino)-5-(4-((2-methylpyridin-4-yl)oxy)phenyl)-2,7-naphthyridin-3-yl)cyclopropanecarboxamide

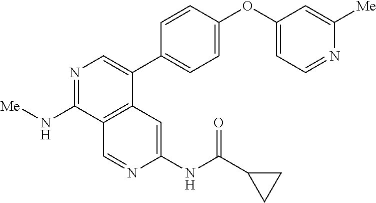 [SMILES: CNc1ncc(-c2ccc(Oc3ccnc(C)c3)cc2)c2cc(NC(=O)C3CC3)ncc12]

### Step 1: 4-(4-bromophenoxy)-2-methylpyridine

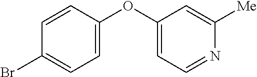 [SMILES: Cc1cc(Oc2ccc(Br)cc2)ccn1]

A solution of 4-iodo-2-methylpyridine (300.0 mg; 1.370 mmol; 1.00 eq.), 4-bromophenol (473.9 mg; 2.740 mmol; 2.00 eq.), (1R,2R)—N¹,N²-dimethylcyclohexane-1,2-diamine (77.9 mg; 0.548 mmol; 0.40 eq.), CuI (52.1 mg; 0.274 mmol; 0.20 eq.) and K₂CO₃ (378.6 mg; 2.740 mmol; 2.00 eq.) in DMSO (9.0 mL) was stirred at 120° C. for 8 hours under nitrogen atmosphere. The desired product was detected via LCMS. The resulting mixture was diluted with EtOAc (50 mL) and washed with brine (3×5 mL). The organic layer was dried over anhydrous Na₂SO₄, concentrated under vacuum. The residue was purified by flash chromatography on pre-packed C18 column using 30-40% of MeCN in water (10 mmol/L NH₄HCO₃) as eluent to provide 4-(4-bromophenoxy)-2-methylpyridine as a yellow solid (219.5 mg, 60.9%). LCMS (ESI) m/z 264.0, [M+H]⁺.

### Step 2: N-(8-(methylamino)-5-(4-((2-methylpyridin-4-yl)oxy)phenyl)-2,7-naphthyridin-3-yl)cyclopropanecarboxamide

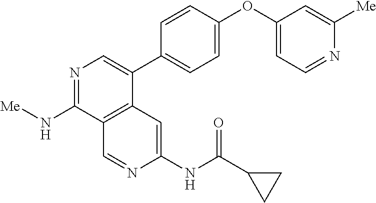 [SMILES: CNc1ncc(-c2ccc(Oc3ccnc(C)c3)cc2)c2cc(NC(=O)C3CC3)ncc12]

A solution of 4-(4-bromophenoxy)-2-methylpyridine (71.0 mg; 0.269 mmol; 1.00 eq.), N-(8-(methylamino)-5-(4,4,5,5-tetramethyl-1,3,2-dioxaborolan-2-yl)-2,7-naphthyridin-3-yl)cyclopropanecarboxamide (120.0 mg; 0.326 mmol; 1.21 eq.), XPhos Pd G₃ (23.0 mg; 0.027 mmol; 0.10 eq.), XPhos (13.0 mg; 0.027 mmol; 0.10 eq.) and K₃PO₄ (172.0 mg; 1.264 mmol; 4.70 eq.) in a mixture solvent of 1,4-dioxane/water (5:1, 6 mL) was stirred at 90° C. for 2 hours under nitrogen atmosphere. The desired product was detected via LCMS. The resulting mixture was concentrated under vacuum and purified by flash chromatography on pre-packed C18 column using 30-50% of MeCN in water (1.0 mmol/L NH₄HCO₃) as eluent to provide N-(8-(methyl amino)-5-(4-((2-methylpyridin-4-yl)oxy)phenyl)-2,7-naphthyridin-3-yl)cyclopropanecarboxamide as a white solid (33.4 mg, 28.0%). LCMS (ESI) m/z 426.2, [M+H]⁺. ¹H NMR (400 MHz, DMSO-d₆) δ 10.95 (s, 1H), 9.39 (s, 1H), 8.36 (d, J=5.6 Hz, 1H), 8.33 (s, 1H), 8.02-7.99 (m, 1H), 7.98 (s, 1H), 7.49 (d, J=8.4 Hz, 2H), 7.27 (d, J=8.4 Hz, 2H), 6.86 (d, J=2.0 Hz, 1H), 6.83 (dd, J=5.6, 2.0 Hz, 1H), 3.02 (d, J=4.4 Hz, 3H), 2.44 (s, 3H), 2.07-1.97 (m, 1H), 0.83-0.73 (m, 4H).

### Example 863: Synthesis of 14-(8-(methylamino)-5-(4-(pyridin-2-yloxy)phenyl)-2,7-naphthyridin-3-yl)cyclopropanecarboxamide

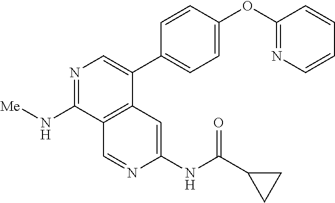 [SMILES: CNc1ncc(-c2ccc(Oc3ccccn3)cc2)c2cc(NC(=O)C3CC3)ncc12]

### Step 1: 2-(4-bromophenoxy)pyridine

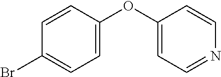 [SMILES: Brc1ccc(Oc2ccncc2)cc1]

A solution of 4-bromophenol (300.0 mg; 1.730 mmol; 1.00 eq.), 2-fluoropyridine (168.1 mg; 1.730 mmol; 1.00 eq.) and K₂CO₃ (718.0 mg; 5.200 mmol; 3.00 eq.) in DMF (3 mL) was stirred at 120° C. overnight under nitrogen atmosphere. The desired product was detected via LCMS. The mixture was diluted with EtOAc (100 mL). The resulting mixture was washed with brine (3×10 mL). The organic layer was dried over anhydrous Na₂SO₄. After filtration, the filtrate was concentrated under reduced pressure. The residue was purified by flash chromatography on silica gel column using 0-15% of EtOAc in petroleum ether as eluent to provide 2-(4-bromophenoxy pyridine) as an off-white solid (110.0 mg, 25.3%). LCMS (ESI) m/z 250.2, [M+H]⁺.

### Step 2: N-(8-(methylamino)-5-(4-(pyridin-2-yloxy)phenyl)-2,7-naphthyridin-3-yl)cyclopropanecarboxamide

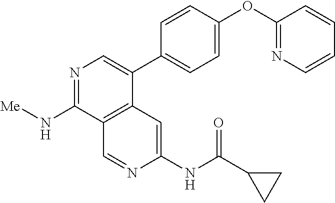 [SMILES: CNc1ncc(-c2ccc(Oc3ccccn3)cc2)c2cc(NC(=O)C3CC3)ncc12]

A solution of N-(8-(methylamino)-5-(4,4,5,5-tetramethyl-1,3,2-dioxaborolan-2-yl)-2,7-naphthyridin-3-yl)cyclopropanecarboxamide (73.6 mg; 0.200 mmol; 1.00 eq.), 2-(4-bromophenoxy)pyridine (50.0 mg; 0.200 mmol; 1.00 eq.), XPhos Pd G₃ (16.9 mg; 0.020 mmol; 0.10 eq.), XPhos (19.0 mg; 0.040 mmol; 0.20 eq.) and K₃PO₄ (84.8 mg; 0.400 mmol; 2.00 eq.) in a mixture solvent of 1,4-dioxane/water (5:1, 2.4 mL) was stirred at 90° C. for 1 hour under nitrogen atmosphere. The desired product was detected via LCMS. The resulting mixture was concentrated under reduced pressure. The residue was purified by flash chromatography on silica gel column using 3-7% of MeOH in CH₂Cl₂ as eluent to afford the crude product. The product was re-purified by flash chromatography on pre-packed CIS column using 20-100% of MeCN in water (10 mmol/L NH₄HCO₃ as eluent to provide N-(8-(methylamino)-5-(4-(pyridin-2-yloxy)phenyl)-2,7-naphthyridin-3-yl)cyclopropanecarboxamide as a white solid (37.0 mg, 44.8%). LCMS (ESI) m/z 412.2, [M+H]⁺. ¹H NMR (400 MHz, DMSO-d₆) δ 10.96 (s, 1H), 9.39 (s, 1H), 8.38 (s, 1H), 8.22 (d, J=1.6 Hz, 1H), 8.01-7.98 (m, 1H), 7.97 (s, 1H), 7.94-7.87 (1H), 7.44 (d, J=8.4 Hz, 2H), 7.23 (d, J=8.4 Hz, 2H), 7.19-7.15 (m, 1H), 7.12-7.06 (m, 1H), 3.02 (d, J=4.4 Hz, 3H), 2.09-1.98 (m, 1H), 0.85-0.75 (m, 4-H).

### Example 864: Synthesis of N-(8-(methylamino)-5-(4-(pyridin-3-yloxy)phenyl)-2,7-naphthyridin-3-yl)cyclopropanecarboxamide

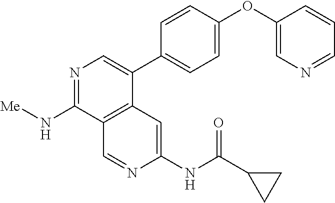 [SMILES: CNc1ncc(-c2ccc(Oc3cccnc3)cc2)c2cc(NC(=O)C3CC3)ncc12]

### Step 1: 3-(4-bromophenoxy)pyridine

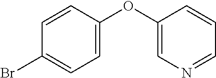 [SMILES: Brc1ccc(Oc2cccnc2)cc1]

To a solution of 4-bromophenol (500.0 mg; 2.890 mmol; 1.00 eq.), pyridin-3-ylboronic acid (1.07 g; 8.705 mmol; 3.01 eq.), Na₂CO₃ (680.0 mg; 6.416 mmol; 2.22 eq.), Cu(OAc)₂ (530.2 mg; 2.919 mmol; 1.01 eq.) in dichloroethane (8 mL) was added 2,2′-bipyridine (455.9 mg; 2.919 mmol; 1.01 eq.). The mixture was stirred at 70° C. for 3 hours under oxygen atmosphere. The reaction was concentrated under pressure. The residue was purified by flash chromatography on silica gel column using 0-25% of EtOAc in petroleum ether as eluent to provide 3-(4-bromophenoxy)pyridine as a light yellow oil (74.0 mg, 8.5%). LCMS (ESI) m/z 250.0, [M+H]⁺.

### Step 2: N-(8-(methylamino)-5-(4-(pyridin-3-yloxy)phenyl)-2,7-naphthyridin-3-yl)cyclopropanecarboxamide

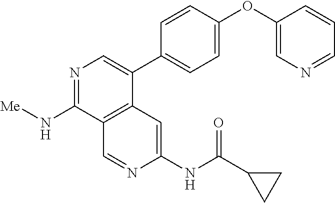 [SMILES: CNc1ncc(-c2ccc(Oc3cccnc3)cc2)c2cc(NC(=O)C3CC3)ncc12]

A solution of N-(8-(methyl amino)-5-(4,4,5,5-tetramethyl-1,3,2-dioxaborolan-2-yl)-2,7-naphthyridin-3-yl)cyclopropanecarboxamide (103.5 mg; 0.281 mmol; 1.00 eq.), 3-(4-bromophenoxy)pyridine (70.0 mg; 0.280 mmol; 1.00 eq.), XPhos Pd G₃ (23.8 mg; 0.028 mmol; 0.10 eq.), XPhos (13.4 mg; 0.028 mmol; 0.10 eq.) and K₃PO₄ (178.9 mg; 0.843 mmol; 3.00 eq.) in a mixture solvent of 1,4-dioxane/water. (5:1, 3 mL) was stirred at 90° C. for 1 hour under nitrogen atmosphere. The reaction was concentrated under pressure. The residue was purified by flash chromatography on silica gel column using 0-25% of MeOH in CH₂Cl₂ as eluent to provide a crude product. The crude product was purified by flash chromatography on pre-packed C18 column using 20-60% of MeCN in water (10 mmol/L NH₄HCO₃) to provide N-(8-(methylamino)-5-(4-(pyridin-3-yloxy)phenyl)-2,7-naphthyridin-3-yl)cyclopropanecarboxamide as an off-white solid (17.2 mg, 14.75%). LCMS (ESI) m/z 412.2, [M+H]⁺. ¹H NMR (400 MHz, DMSO-d₆) δ 10.95 (s, 1H), 9.38 (s, 1H), 8.47 (d, J=2.8 Hz, 1H), 8.40-8.44 (m, 1H), 8.34 (s, 1H), 8.02-7.95 (m, 1H), 7.94 (s, 1H), 7.59-7.52 (m, 1H), 7.52-7.47 (m, 1H), 7.44 (d, J=8.4 Hz, 2H), 7.16 (d, J=8.4 Hz, 2H), 3.01 (d, J=4.4 Hz, 3H), 2.11-1.92 (m, 1H), 0.89-0.71 (m, 4H).

### Example 865: Synthesis of N-(5-(4-((1-methyl-1H-1,2,4-triazol-3-yl)oxy)phenyl)-8-(methylamino)-2,7-naphthyridin-3-yl)cyclopropanecarboxamide

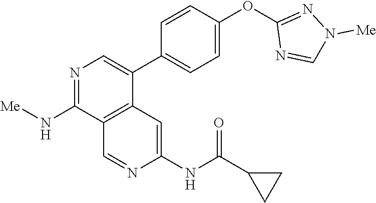 [SMILES: CNc1ncc(-c2ccc(Oc3ncn(C)n3)cc2)c2cc(NC(=O)C3CC3)ncc12]

### Step 1: 3-(4-bromophenoxy)-1-methyl-1H-1,2,4-triazole

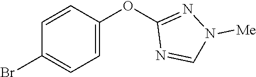 [SMILES: Cn1cnc(Oc2ccc(Br)cc2)n1]

In a microwave reaction vial (25 mL), a solution of 4-bromophenol (500.0 mg; 2.890 mmol; 1.00 eq.), 3-bromo-1-methyl-1H-1,2,4-triazole (936.4 mg; 5.781 mmol; 2.00 eq.) and i-BuOK (388.4 mg; 3.461 mmol; 1.20 eq.) in DMSO (7 mL) was stirred at 180° C. for 2 hours in a microwave reactor. The resulting mixture was diluted with EtOAc (100 mL) and washed with brine (3×20 mL). The organic layer was dried over anhydrous Na₂SO₄. The resulting mixture was filtered, the filtrate was concentrated under reduced pressure. The residue was purified by flash chromatography on pre-packed C18 column using 20-60% of MeCN in water (10 mmol/L NH₄HCO₃) as eluent to provide 3-(4-bromophenoxy)-1-methyl-1H-1,2,4-triazole as a brown yellow oil (24 mg, 3.1%). LCMS (ESI) m/z 254.0, [M+H]⁺.

### Step 2: N-(5-(4-((1-methyl-1H-1,2,4-triazol-3-yl)oxy)phenyl)-8-(methylamino)-2,7-naphthyridin-3-yl)cyclopropanecarboxamide

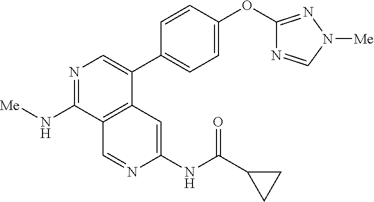 [SMILES: CNc1ncc(-c2ccc(Oc3ncn(C)n3)cc2)c2cc(NC(=O)C3CC3)ncc12]

A solution of N-(8-(methyl amino)-5-(4,4,5,5-tetramethyl-1,3,2-dioxaborolan-2-yl)-2,7-naphthyridin-3-yl)cyclopropanecarboxamide (34.9 mg; 0.095 mmol; 1.00 eq.), 3-(4-bromophenoxy)-1-methyl-1H-1,2,4-triazole (24.0 mg; 0.094 mmol; 1.00 eq.), XPhos Pd G₃ (8.0 mg; 0.009 mmol; 0.10 eq.), XPhos (4.5 mg; 0.009 mmol; 0.10 eq.) and K₃PO₄ (60.3 mg; 0.284 mmol; 3.00 eq.) in a mixture solvent of 1,4-dioxane/water (5:1, 1.2 mL) was stirred at 90° C. for 1 hour. The reaction was concentrated under pressure. The residue was purified by flash chromatography on silica gel column using 0-25% of MeOH in CH₂Cl₂ as eluent to provide the crude product. The crude product was purified by flash chromatography on pre-packed C18 column using 20-100% of MeCN in water. (10 mmol/L NH₄HCO₃) as eluent to provide N-(5-(4-((1-methyl-1H-1,2,4-triazol-3-yl)oxy)phenyl)-8-(methylamino)-2,7-naphthyridin-3-yl)cyclopropanecarboxamide as an off-white solid (8.0 mg, 20.2%). LCMS (ESI) m/z 416.2, [M+H]⁺. ¹H NMR (400 MHz, DMSO-d₆) δ 10.94 (s, 1H), 9.38 (s, 1H), 8.34 (s, 2H), 8.01-7.94 (m, 1H), 7.93 (s, 1H), 7.46-7.38 (m, 2H), 7.32-7.24 (m, 2H), 3.81 (s, 3H), 3.01 (d, J=4.4 Hz, 3H), 2.10-1.91 (m, 1H), 0.90-0.70 (m, 4H).

### Example 866: Synthesis of N-(5-(2-(1-methyl-1H-benzo[d][1,2,3]triazol-6-yl)ethyl)-8-(methylamino)-2,7-naphthyridin-3-yl)cyclopropanecarboxamide

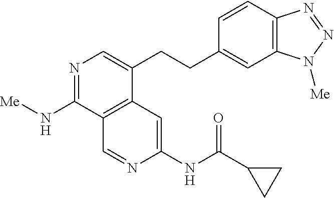 [SMILES: CNc1ncc(CCc2ccc3nnn(C)c3c2)c2cc(NC(=O)C3CC3)ncc12]

### Step 1: (E)-N-(5-(2-(1-methyl-1H benzo[d][1,2,3]triazol-6-yl)vinyl)-8-(methylamino)-2,7-naphthyridin-3-yl)cyclapropanecarboxamide

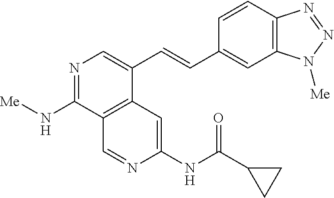 [SMILES: CNc1ncc(/C=C/c2ccc3nnn(C)c3c2)c2cc(NC(=O)C3CC3)ncc12]

To a solution of N-(8-(methylamino)-5-vinyl-2,7-naphthyridin-3-yl)cyclopropanecarboxamide (68.0 mg; 0.253 mmol; 1.00 eq.) in DMF (2 mL) was added 6-bromo-1-methyl-1H-benzo[d][1,2,3]triazole (160.0 mg; 0.755 mmol; 2.98 eq.), Pd(PPh₃)₂Cl₂(18.0 mg; 0.026 mmol; 0.10 eq.), Na₂CO₃ (54.0 mg; 0.509 mmol; 2.01 eq.) under nitrogen atmosphere. The resulting solution was stirred at 100° C. for 12 hours. The resulting mixture was diluted with water (10 mL) and extracted with EtOAc (3×20 mL). The combined organic layers were washed with brine (3×10 mL), dried over anhydrous Na₂SO₄. After filtration, the filtrate was concentrated under reduced pressure and purified by flash chromatography on silica gel column using 5-10% of MeOH in CH₂Cl₂ as eluent to provide (E)-N-(5-(2-(1-methyl-1H-benzo[d][1,2,3]triazol-6-yl)vinyl)-8-(methylamino)-2,7-naphthyridin-3-yl)cyclopropanecarboxamide as a yellow solid (90 mg, 88.9%). LCMS (ESI) m/z 400.2, [M+H]⁺.

### Step 2: N-(5-(2-(1-methyl-1H-benzo[d][1,2,3]triazol-6-yl)ethyl)-8-(methylamino)-2,7-naphthyridin-3-yl)cyclopropanecarboxamide

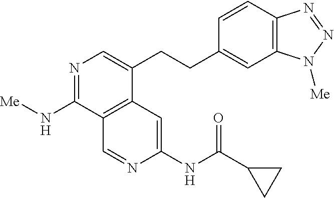 [SMILES: CNc1ncc(CCc2ccc3nnn(C)c3c2)c2cc(NC(=O)C3CC3)ncc12]

To a solution of (E)-N-(5-(2-(1-methyl-1H-benzo[d][1,2,3]triazol-6-yl)vinyl)-8-(methylamino)-2,7-naphthyridin-3-yl)cyclopropanecarboxamide (110.0 mg; 0.275 mmol, 1.00 eq.) in MeOH (10 mL) was added 10% Pd/C (110 mg; 10% w/w) under nitrogen atmosphere. The mixture was hydrogenated at room temperature for 12 hours under hydrogen atmosphere (2 atm). After filtration, the filtrate was concentrated under reduced pressure. The residue was purified by flash chromatography on silica gel column using 5-10% of MeOH in CH₂Cl₂ as eluent to provide N-(5-(2-(1-methyl-1H-benzo[d][1,2,3]triazol-6-yl)ethyl)-8-(methyl amino)-2,7-naphthyridin-3-yl)cyclopropanecarboxamide as a yellow solid (19.2 mg, 17.3%). LCMS (ESI) m/z 402.2, [M+H]⁺. ¹H NMR (400 MHz, DMSO-d₆) δ 11.07 (s, 1H), 9.35 (s, 1H), 8.50 (s, 1H), 7.93 (d, J=8.4 Hz, 1H), 7.85-7.78 (m, 2H), 7.30 (dd, J=8.8, 1.6 Hz, 1H), 4.29 (s, 3H), 3.13-2.97 (m, 4H), 2.94 (d, J=4.4 Hz, 3H), 2.15-2.04 (m, 1H), 0.92-0.82 (m, 4H).

### Example 867: Synthesis of N-(8-(methylamino)-5-(4-(pyridin-4-ylmethoxy)phenyl)-2,7-naphthyridin-3-yl)cyclopropanecarboxamide

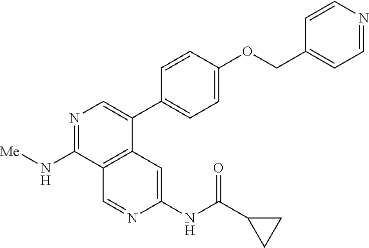 [SMILES: CNc1ncc(-c2ccc(OCc3ccncc3)cc2)c2cc(NC(=O)C3CC3)ncc12]

### Step 1: 4-((4-bromophenoxy)methyl)pyridine

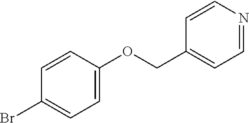 [SMILES: Brc1ccc(OCc2ccncc2)cc1]

To a solution of 4-bromophenol (500.0 mg; 2.890 mmol; 1.00 eq.) in DMF (10 mL) were added Cs₂CO₃ (2.8 g; 8.699 mmol; 3.01 eq.) and 4-(chloromethyl)pyridine (738.7 mg; 5.780 mmol; 2.00 eq.) at 0° C. The reaction was stirred at 50° C. for 2 hours under nitrogen atmosphere. The desired product was detected via LCMS. The resulting mixture was diluted with EtOAc (100 mL) and washed by brine (3×15 mL). The organic layer was dried over anhydrous sodium sulfate and concentrated under vacuum. The residue was purified by flash chromatography on silica gel column using 20-70% of EtOAc in petroleum ether as eluent to provide 4-((4-bromophenoxy)methyl)pyri dine as a white oil (550.0 mg, 72.0%). LCMS (ESI) m/z 264.0, [M+H]⁺.

### Step 2: N-(8-(methylamino)-5-(4-(pyridin-4-ylmethoxy)phenyl)-2,7-naphthyridin-3-yl)cyclopropanecarboxamide

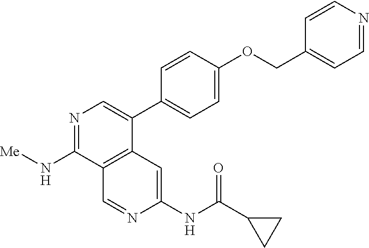 [SMILES: CNc1ncc(-c2ccc(OCc3ccncc3)cc2)c2cc(NC(=O)C3CC3)ncc12]

To a solution of 4-((4-bromophenoxy)methyl)pyridine (107.0 mg; 0.405 mmol; 1.00 eq.) in a mixture solvent of 1,4-dioxane/water (5:1, 6 mL) was added XPhos Pd G₃ (34.4 mg; 0.041 mmol; 0.10 eq.), XPhos (19.4 mg; 0.041 mmol; 0.10 eq.), K₃PO₄ (259.0 mg; 1.220 mmol; 3.00 eq.) and N-(8-(methylamino)-5-(4,4,5,5-tetramethyl-1,3,2-dioxaborolan-2-yl)-2,7-naphthyridin-3-yl) cyclopropanecarboxamide (150.0 mg; 0.405 mmol; 1.00 eq.). The reaction was stirred at 90° C. for 1 hour under nitrogen atmosphere. The desired product was detected via LCMS. The resulting mixture was concentrated under vacuum and purified by flash chromatography on silica gel column using 2-10% of MeOH in CH₂Cl₂ as eluent to provide the crude product. The product was re-purified by flash chromatography on pre-packed C18 column using 20-80% of MeCN in water (10 mmol/L NH₄HCO₃) as eluent to provide N-(8-(methylamino)-5-(4-(pyridin-4-ylmethoxy)phenyl)-2,7-naphthyridin-3-yl)cyclopropanecarboxamide as a white solid (10.2 mg, 5.9%). LCMS (ESI) m/z 426.2, [M+H]⁺. ¹H NMR (400 MHz, DMSO-d₆) δ 10.93 (s, 1H), 9.37 (s, 1H), 8.65-8.55 (m, 2H), 8.32 (s, 1H), 7.96-7.90 (m, 1H), 7.89 (s, 1H), 7.55-7.44 (m, 2H), 7.34 (d, J=8.4 Hz, 2H), 7.14 (d, J=8.4 Hz, 2H), 5.26 (s, 2H), 3.00 (d, J=4.4 Hz, 3H), 2.07-1.97 (m, 1H), 0.82-0.76 (m, 4H).

### Example 868: Synthesis of N-(8-(methylamino)-5-(4-((pyridin-4-yloxy)methyl)phenyl)-2,7-naphthyridin-3-yl)cyclapropanecarboxamide

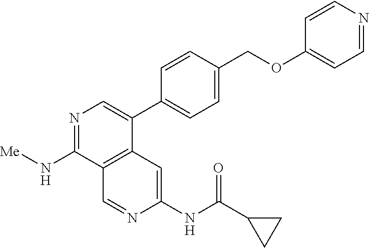 [SMILES: CNc1ncc(-c2ccc(COc3ccncc3)cc2)c2cc(NC(=O)C3CC3)ncc12]

### Step 1: 4-((4-bromobenzyl)oxy)pyridine

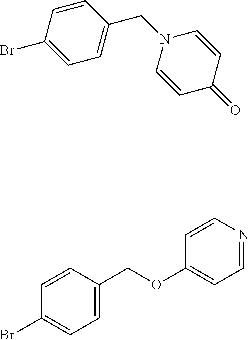 [SMILES: O=c1ccn(Cc2ccc(Br)cc2)cc1 ; Brc1ccc(COc2ccncc2)cc1]

To a stirred solution of 1-bromo-4-(bromomethyl)benzene (500.0 mg; 2.001 mmol; 1.00 eq.) and pyridin-4-ol (190.2 mg; 2.001 mmol; 1.00 eq.) in DMF (5 mL) was added Cs₂CO₃ (1303.6 mg; 4.002 mmol; 2.00 eq.) at room temperature. The resulting mixture was stirred at room temperature for 2 hours. The desired product was detected via LCMS. The mixture was diluted with EtOAc (30 mL) and washed with water (3×10 mL). The organic layer was dried over anhydrous Na₂SO₄. After filtration, the filtrate was concentrated under reduced pressure. The residue was purified by flash chromatography on pre-packed C18 column using 20-50% of MeCN in water (10 mmol/L NH₄HCO₃) to afford 1-(4-bromobenzyl)pyridin-4(1H)-one as a brown solid (260.0 mg, 49.2%) and 4-((4-bromobenzyl)oxy)pyridine as an orange solid (110.0 mg, 52.1%). LCMS (ESI) m/z 264.0, [M+H]⁺.

### Step 2: N-(8-(methylamino)-5-(4-((pyridin-4-yloxy)methyl)phenyl)-2,7-naphthyridin-3-yl)cyclopropanecarboxamide

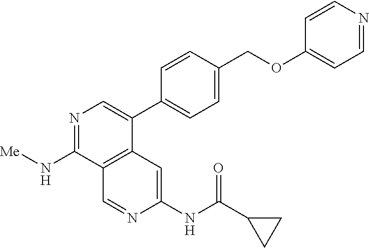 [SMILES: CNc1ncc(-c2ccc(COc3ccncc3)cc2)c2cc(NC(=O)C3CC3)ncc12]

To a solution of 4-((4-bromobenzyl)oxy)pyridine (71.7 mg; 0.272 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (100.0 mg; 0.272 mmol; 1.00 eq.) in a mixture solvent of 1,4-dioxane/water (5:1, 2.5 mL) were added XPhos Pd G₃ (23.0 mg; 0.027 mmol; 0.10 eq.), XPhos (25.9 mg; 0.054 mmol; 0.20 eq.) and K₃PO₄ (144.1 mg; 0.680 mmol; 2.50 eq.) under nitrogen atmosphere. The resulting mixture was stirred at 90° C. for 1 hour. The desired product was detected via LCMS. The solvent was concentrated under vacuum. The residue was purified by flash chromatography on silica gel column using 1-9% of MeOH in CH₂Cl₂ as eluent to provide the crude product. The crude product was purified by flash chromatography on pre-packed C18 column using 10-40% of MeCN in water (10 mmol/L NH₄HCO₃) to provide N-(8-(methylamino)-5-(4-((pyridin-4-yloxy)methyl)phenyl)-2,7-naphthyridin-3-yl)cyclopropanecarboxamide as a white solid (18.6 mg, 16.1%). LCMS (ESI) m/z 426.2, [M+H]⁺. ¹H NMR (400 MHz, DMSO-d₆) δ 10.97 (s, 1H), 9.39 (s, 1H), 8.46-8.40 (m, 2H), 8.38 (s, 1H), 8.04-7.97 (m, 1H), 7.95 (s, 1H), 7.61-7.55 (m, 2H), 7.49-7.43 (m, 2H), 7.14-7.08 (m, 2H), 5.27 (s, 2H), 3.01 (d, J=4.4 Hz, 3H), 2.07-1.97 (m, 1H), 0.81-0.76 (m, 4H).

### Example 869: Synthesis of N-(8-(methylamino)-5-(4-((4-oxopyridin-1(4H)-yl)methyl)phenyl)-2,7-naphthyridin-3-yl)cyclapropanecarboxamide

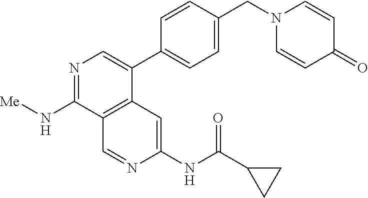 [SMILES: CNc1ncc(-c2ccc(Cn3ccc(=O)cc3)cc2)c2cc(NC(=O)C3CC3)ncc12]

To a solution of 1-(4-bromobenzyl)pyridin-4(1H)-one (Example 868, step 1) (71.7 mg; 0.272 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (100.0 mg; 0.272 mmol; 1.00 eq.) in a mixture solvent of 1,4-dioxane/water (5:1, 2.5 mL) were added XPhos Pd G₃ (23.0 mg; 0.027 mmol; 0.10 eq.), XPhos (25.9 mg; 0.054 mmol; 0.20 eq.) and K₃PO₄ (144.1 mg; 0.680 mmol; 2.50 eq.) at room temperature under nitrogen atmosphere. The resulting mixture was stirred at 90° C. for 1 hour. The desired product was detected via LCMS. The mixture was allowed to cool down to room temperature. The solvent was concentrated under reduced pressure and purified by flash chromatography on silica gel column using 2-8% of MeOH in CH₂Cl₂ as eluent to provide the crude product. The crude product was purified by flash chromatography on pre-packed C18 column using 20-60% of MeCN in water. (10 mmol/L NH₄HCO₃) to provide N-(8-(methylamino)-5-(4-((4-oxopyridin-1(4H)-yl)methyl)phenyl)-2,7-naphthyridin-3-yl)cyclopropanecarboxamide as a white solid (20.3 mg, 17.3%). LCMS (ESI) m/z 426.2, [M+H]⁺.

### Example 870: Synthesis of N-(5-(4-(1-methyl-1H-1,2,3-triazol-5-yl)phenyl)-8-(methylamino)-2,7-naphthyridin-3-yl)cyclopropanecarboxamide

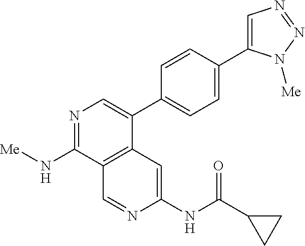 [SMILES: CNc1ncc(-c2ccc(-c3cnnn3C)cc2)c2cc(NC(=O)C3CC3)ncc12]

### Step 1: 5-1H-bromophenyl)-1H-1,2,3-triazole

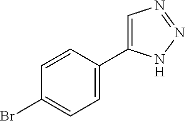 [SMILES: Brc1ccc(-c2cnn[nH]2)cc1]

To a solution of 1-bromo-4-ethynylbenzene (500.0 mg; 2.762 mmol; 1.00 eq.) in xylene (8 mL) was added azidotrimethylsilane (1.27 g; 11.1.03 mmol; 4.02 eq.) under nitrogen atmosphere. The reaction was stirred at 140° C. overnight. The desired product was detected via LCMS. The mixture was purified by flash chromatography on pre-packed C18 column using 20-40% of MeCN in water (10 mmol/L NH₄HCO₃) to provide 5-(4-bromophenyl)-1H-1,2,3-triazole as white solid (260 mg, 40.7%). LCMS (ESI) m/z 224.0, [M+H]⁺.

### Step 2: 5-(4-bromophenyl)-1-methyl-1H-1,2,3-triazole and 4-(4-bromophenyl)-1-methyl-1H-1,2,3-triazole

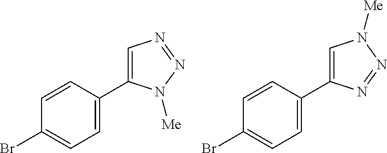 [SMILES: Cn1nncc1-c1ccc(Br)cc1 ; Cn1cc(-c2ccc(Br)cc2)nn1]

To a solution of 5-(4-bromophenyl)-1H-1,2,3-triazole (210.0 mg; 0.937 mmol; 1.00 eq.), Cu(OAc)₂ (171.9 mg; 0.946 mmol; 1.01 eq.) and Na₂CO₃ (219.5 mg; 2.071 mmol; 2.21 eq.) in dichloroethane (5 mL) were added 2,2′-bipyridine (146.4 mg; 0.937 mmol; 1.00 eq.) and 2,4,6-trimethyl-1,3,5,2,4,6-trioxatriborinane (1.41 g; 5.649 mmol; 6.03 eq.). The reaction was stirred at 70° C. for 3 hours. The desired product was detected via LCMS. The mixture was concentrated under reduced pressure and purified by flash chromatography on silica gel column using 0-25% of EtOAc in petroleum ether as eluent to provide 5-(4-bromophenyl)-1-methyl-1H-1,2,3-triazole as an off-white solid (35.0 mg, 11.5%) and 4-(4-bromophenyl)-1-methyl-1H-1,2,3-triazole as an off-white solid (42.0 mg, 18.8%). LCMS (ESI) m/z 238.0, [M+H]⁺.

### Step 3: N-(5-(4-(1-methyl-1H-1,2,3-triazol-5-yl)phenyl)-8-(methylamino)-2,7-naphthyridin-3-yl)cyclopropanecarboxamide

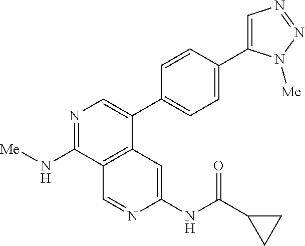 [SMILES: CNc1ncc(-c2ccc(-c3cnnn3C)cc2)c2cc(NC(=O)C3CC3)ncc12]

A solution of 5-(4-bromophenyl)-1-methyl-1/1-1,2,3-triazole (43.0 mg; 0.181 mmol, 1.00 eq.), N-(8-(methylamino)-5-(4,4,5,5-tetramethyl-1,3,2-dioxaborolan-2-yl)-2,7-naphthyridin-3-yl)cyclopropanecarboxamide (67.6 mg; 0.183 mmol; 1.01 eq.), XPhos Pd G₃ (15.3 mg; 0.018 mmol; 0.10 eq.), XPhos (8.6 mg; 0.018 mmol; 0.10 eq.) and K₃PO₄ (115.4 mg; 0.545 mmol; 3.01 eq.) in a mixture solvent of 1,4-dioxane/water (5:1, 3 mL) was stirred at 90° C. for 1 hour under nitrogen atmosphere. The reaction was concentrated under pressure and purified by flash chromatography on silica gel column using 0-25% of MeOH in CH₂Cl₂ as eluent to provide the crude product. The crude product was purified by Prep-.Achiral-SFC (Column: Torus 2-P1C OBD, 186008592; Mobile Phase A: CO₂, Mobile Phase B: MeOH (0.1% 2 M NH₃-MeOH); Flow rate: 60 mL/min Gradient: isocratic 33% B; Column Temperature (° C.): 35; Back Pressure (bar): 100; Wave Length: 254 nm) to afford N-(5-(4-(1-methyl-1H-1,2,3-triazol-5-yl)phenyl)-8-(methylamino)-2,7-naphthyridin-3-yl)cyclopropanecarboxamide as an off-white solid (14.6 mg, 20.1%). LCMS (ESI) m/z 400.2, [M+H]⁺. ¹H NMR (400 MHz, DMSO-d₆) 10.97 (s, 1H), 9.41 (s, 1H), 8.41 (s, 1H), 8.08-8.02 (m, 1H), 8.01 (s, 1H), 8.00 (s, 7.75 (d, J=8.4 Hz, 2H), 7.60 (d, J=8.4 Hz, 2H), 4.16 (s, 3H), 3.03 (d, J=4.4 Hz, 3H), 2.10-1.95 (m, 1H), 0.83-0.75 (m, 4H).

### Example 871: Synthesis of N-(5-(4-(1-methyl-1H-1,2,3-triazol-4-yl)phenyl)-8-(methylamino)-2,7-naphthyridin-3-yl)cyclopropanecarboxamide

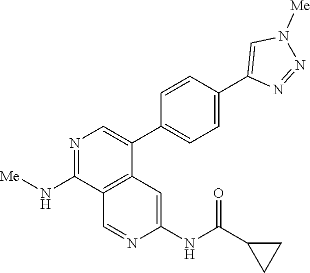 [SMILES: CNc1ncc(-c2ccc(-c3cn(C)nn3)cc2)c2cc(NC(=O)C3CC3)ncc12]

A solution of 4-(4-bromophenyl)-1-methyl-1H-1,2,3-triazole (Example 870, step 2) (73.0 mg; 0.307 mmol; 1.00 eq.), N-(8-(methylamino)-5-(4,4,5,5-tetramethyl-1,3,2-dioxaborolan-2-yl)-2,7-naphthyridin-3-yl)cyclopropanecarboxamide (112.3 mg; 0.304 mmol; 1.01 eq.), XPhos Pd (13 (25.6 mg; 0.031 mmol; 0.10 eq.), XPhos (14.6 mg; 0.031 mmol; 0.10 eq.) and K₃PO:1 (195.9 mg; 0.924 mmol; 3.01 eq.) in a mixture solvent of 1,4-dioxane/water (5:1, 3 mL) was stirred at 90° C. for 1 hour under nitrogen atmosphere. The desired product was detected via LCMS. The reaction was concentrated under reduced pressure and purified by flash chromatography on silica gel column using 0-25% of MeOH: in CH₂Cl₂ as eluent to provide the crude product. The crude product was purified by flash chromatography on pre-packed C18 column using 20-60% of MeCN in water (10 mmol/L NH₄HCO₃) to provide N-(5-(4-(1-methyl-1H-1,2,3-triazol-4-yl)phenyl)-8-(methylamino)-2,7-naphthyridin-3-yl)cyclopropanecarboxamide as an off-white solid (56.8 mg, 45.8%). LCMS (ESI) m/z 400.2, [M+H]⁺. ¹H NMR (400 MHz, DMSO-d₆) δ 10.96 (s, 1H), 9.40 (s, 1H), 8.41 (s, 1H), 8.30 (s, 1H), 8.04-8.00 (m, 1H), 7.99 (s, 1H), 7.94 (d, J=8.4 Hz, 2H), 7.50 (d, J=8.4 Hz, 2H), 4.23 (s, 3H), 3.02 (d, J=4.4 Hz, 3H), 2.08-1.95 (m, 1H), 0.91-0.73 (m, 4H).

### Example 872: Synthesis of N-(5-(imidazo[2,1-b][1,3,4]thiadiazol-2-yl)-8-(methylamino)-2,7-naphthyridin-3-yl)cyclopropanecarboxamide

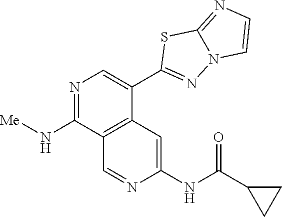 [SMILES: CNc1ncc(-c2nn3ccnc3s2)c2cc(NC(=O)C3CC3)ncc12]

To a solution of N-(8-(methylamino)-5-(4,4,5,5-tetramethyl-1,3,2-dioxaborolan-2-yl)-2,7-naphthyridin-3-yl)cyclopropanecarboxamide (200.0 mg; 0.543 mmol; 1.00 eq.) and 2-bromoimidazo[2,1-b][1,3,4]thiadiazole (110.0 mg; 0.539 mmol; 0.99 eq.) in a mixture solvent of dioxane/water (5:1, 4.8 mL) were added XPhos Pd (13 (92.0 mg; 0.109 mmol; 0.20 eq.), XPhos (52.0 mg; 0.109 mmol; 0.20 eq.) and K₃PO₄ (352.0 mg; 1.658 mmol; 3.05 eq.) under nitrogen atmosphere. The resulting solution was stirred at 90° C. for 1 hour. The resulting mixture was concentrated under reduced pressure. The residue was dissolved in EtOAc (50 mL) and washed with brine (3×10 mL). The organic layer was dried over anhydrous Na₂SO₄. After filtration, the filtrate was concentrated under reduced pressure. The residue was purified by flash chromatography on silica gel column using 5-10% of MeOH in CH₂Cl₂ as eluent to provide N-(5-(imidazo[2,1-b][1,3,4]thiadiazol-2-yl)-8-(methylamino)-2,7-naphthyridin-3-yl)cyclopropanecarboxamide as a yellow solid (13.5 mg, 6.8%). LCMS (ESI) m/z 366.1, ¹H NMR (400 MHz, DMSO-d₆) δ 11.12 (s, 1H), 9.45 (s, 1H), 9.14 (s, 1H), 8.69-8.63 (m, 1H), 8.48 (s, 1H), 8.19 (d, J=1.6 Hz, 1H), 7.35 (d, J=1.6 Hz, 1H), 3.07 (d, J=4.4 Hz, 3H), 2.11-2.04 (m, 1H), 0.90-0.80 (m, 4H).

### Example 873: Synthesis of N-(8-(methylamino)-5-(5-methylimidazo[5,1-b][1,3,4]oxadiazol-2-yl)-2,7-naphthyridin-3-yl)cyclopropanecarboxamide

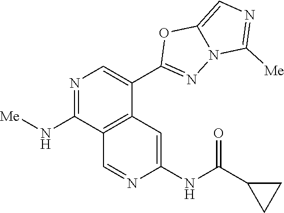 [SMILES: CNc1ncc(-c2nn3c(C)ncc3o2)c2cc(NC(=O)C3CC3)ncc12]

### Step 1: methyl 6-(cyclopropanecarboxamido)-1-(methylamino)-2,7-naphthyridine-4-carboxylate

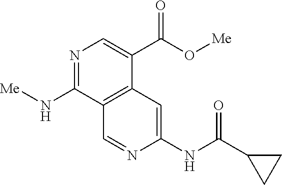 [SMILES: CNc1ncc(C(=O)OC)c2cc(NC(=O)C3CC3)ncc12]

To a solution of N-(5-bromo-8-(methylamino)-2,7-naphthyridin-3-yl)cyclopropanecarboxamide (3.00 g; 9.340 mmol; 1.00 eq.) in a mixture solvent of methanol/DMSO (1:2, 27 mL) was added Pd(dppf)Cl₂·CH₂Cl₂ (760.9 mg; 0.934 mmol; 0.1.0 eq.) and triethylamine (2.84 g; 28.060 mmol; 3.00 eq.) in a pressure tank. The mixture was purged with nitrogen for 1 min and pressurized to 20 atm with carbon monoxide at 130° C. for 1.4 hours. The reaction mixture was cooled to room temperature. The desired product was detected via LCMS. The reaction solution was diluted with EtOAc (300 mL), washed with brine (3×50 mL), dried over Na₂SO₄. After filtration, the filtrate was concentrated under reduced pressure. The residue was purified by flash chromatography on silica gel column using 60-90% of EtOAc in petroleum ether as eluent to provide methyl 6-(cyclopropanecarboxamido)-1-(methylamino)-2,7-naphthyridine-4-carboxylate as a yellow solid (2.80 g, 99%). LCMS (ESI) m/z 301.1, [M+H]⁺.

### Step 2: N-(5-(hydrazinecarbonyl)-8-(methylamino)-2,7-naphthyridin-3-yl)cyclopropanecarboxamide

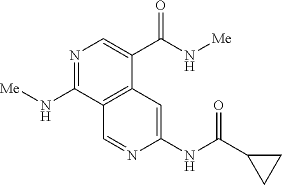 [SMILES: CNC(=O)c1cnc(NC)c2cnc(NC(=O)C3CC3)cc12]

To a stirred solution of methyl 6-(cyclopropanecarboxamido)-1-(methylamino)-2,7-naphthyridine-4-carboxylate (200.0 mg; 0.666 mmol; 1.00 eq.) in MeOH (20 mL) was added hydrazine hydrate (80%, 2.0 mL) dropwise at room temperature. The resulting mixture was stirred at 60° C. for 14 hours under nitrogen atmosphere. The precipitated solids were collected by filtration and washed with MeOH (20 mL) just once to afford N-(5-(hydrazinecarbonyl)-8-(methylamino)-2,7-naphthyridin-3-yl)cyclopropanecarboxamide (115.1 mg, 57.5%) as an off-white solid. LCMS (ESI) m/z 301.1, [M+H]⁺.

### Step 3: N-(5-(2-(acetylglycyl)hydrazine-1-carbonyl)-8-(methylamino)-2,7-naphthyridin-3-yl)cyclopropanecarboxamide

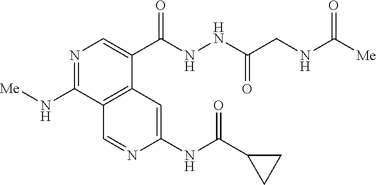 [SMILES: CNc1ncc(C(=O)NNC(=O)CNC(C)=O)c2cc(NC(=O)C3CC3)ncc12]

To a solution of acetylglycine (117.2 mg; 1.001 mmol; 2.00 eq.) in DMF (3 mL) was added CDI (178.5 mg; 1.101 mmol; 2.20 eq.). The resulting mixture was stirred at room temperature until no gas evolution was observed. To the mixtures was added N-(5-(hydrazinecarbonyl)-8-(methylamino)-2,7-naphthyridin-3-yl)cyclopropanecarboxamide (150.3 mg; 0.500 mmol; 1.00 eq.) and the reaction was stirred at room temperature overnight. The desired product was detected via LCMS. The mixture was purified by flash chromatography on pre-packed C18 column using 30-100% of MeCN in water (10 mmol/L NH₄HCO₃) as eluent to provide N-(5-(2-(acetylglycyl)hydrazine-1-carbonyl)-8-(methylamino)-2,7-naphthyridin-3-yl)cyclopropanecarboxamide as a yellow solid (151.8 mg, 75.9%). LCMS (ESI) m/z 400.2, [M+H]⁺.

### Step 4: N-(8-(methylamino)-5-(5-methylimidazo[5,1-b][1,3,4]oxadiazol-2-yl)-2,7-naphthyridin-3-yl)cyclopropanecarboxamide

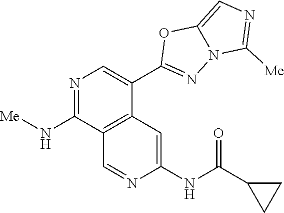 [SMILES: CNc1ncc(-c2nn3c(C)ncc3o2)c2cc(NC(=O)C3CC3)ncc12]

To a stirred solution of N-(5-(2-(acetylglycyl)hydrazine-1-carbonyl)-8-(methylamino)-2,7-naphthyridin-3-yl)cyclopropanecarboxamide (92.4 mg; 0.231 mmol; 1.00 eq.) in acetonitrile (8 mL) was added POCl₃ (35.4 mg; 0.231 mmol; 1.00 eq.) dropwise at 0° C. The resulting mixture was stirred at 90° C. for 3.5 hours under nitrogen atmosphere. The desired product was detected via LCMS. The reaction was quenched by the addition of water (0.2 mL) at room temperature. The mixture was purified by flash chromatography on pre-packed C18 column using 30-1.00% of MeCN in water (10 mmol/L NH₄HCO₃) as eluent to provide N. (8-(methylamino)-5-(5-methylimidazo[5,1-b][1,3,4]oxadiazol-2-yl)-2,7-naphthyridin-3-yl)cyclopropanecarboxamide as an off-white solid (16.8 mg, 19.9%). LCMS (ESI) m/z 364.1, [M+H]⁺. ¹H NMR (400 MHz, DMSO-d₆) δ 11.08 (s, 1H), 9.44 (s, 1H), 9.35 (s, 1H), 8.81-8.76 (m, 1H), 8.75 (s, 1H), 6.50 (s, 1H), 3.09 (d, J=4.4 Hz, 3H), 2.47 (s, 3H), 2.15-2.03 (m, 1H), 0.96-0.79 (m, 4H).

### Example 874: Synthesis of N-(5-(5-methoxy-4,5,6,7-tetrahydrobenzo[d]oxazol-2-yl)-8-(methylamino)-2,7-naphthyridin-3-yl)cyclopropanecarboxamide

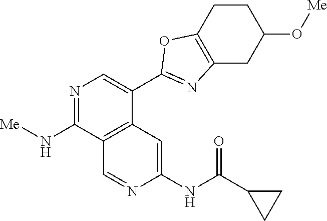 [SMILES: CNc1ncc(-c2nc3c(o2)CCC(OC)C3)c2cc(NC(=O)C3CC3)ncc12]

### Step 1: 4-methoxycyclohex-1-ene

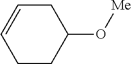

To a solution of 3-cyclohexen-1-ol (500.0 mg; 5.095 mmol; 1.00 eq.) in THF (10 mL) was added NaH (60%) (509.4 mg; 1.2.736 mmol; 2.50 eq.) at 0° C. The mixture was stirred 0° C. for 0.5 hour. To the above solution was added methyl iodide (1.45 g; 10.190 mmol; 2.00 eq.) dropwise. The reaction was stirred at room temperature for 2 hours. The reaction was monitored by TLC (EtOAc/petroleum ether=1:2) until the starting material was consumed completely. The reaction solution was diluted with CH₂Cl₂ (50 mL), washed with brine (2×20 mL). The organic phase was dried over Na₂SO₄. After filtration, the filtrate was concentrated under reduced pressure to provide 4-methoxycyclohex-1-ene as a yellow oil (570 mg, 99.7%).

### Step 2: 3-methoxy-7-oxabicyclo[4.1.0]heptane

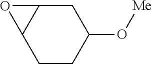

A solution of 4-methoxycyclohex-1-ene (571.4 mg; 5.094 mmol; 1.00 eq.) and m-CPBA (1.76 g; 10.188 mmol; 2.00 eq.) in CH₂Cl₂ (10 mL) was stirred overnight at room temperature. The reaction was monitored by TLC (EtOAc/petroleum ether=1:2) until the starting material was consumed completely. The reaction solution was diluted with CH₂Cl₂ (50 mL), washed with saturated Na₂S₂O₃·5H₂O (2×10 mL) and saturated NaHCO₃ solution (2×10 mL). The organic phase was dried over Na₂SO₄. After filtration, the filtrate was concentrated under reduced pressure to provide 3-methoxy-7-oxabicyclo[4.1.0]heptane as a yellow oil (794 mg, crude).

### Step 3: 2-(benzylamino)-4-methoxycyclohexan-1-ol

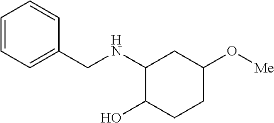 [SMILES: COC1CCC(O)C(NCc2ccccc2)C1]

A solution of 3-methoxy-7-oxabicyclo[4.1.0]heptane (794.0 mg; 6.195 mmol; 1.00 eq.) and benzylamine (1.33 g; 12.390 mmol; 2.00 eq.) in i-PrOH (10 mL) was stirred at 90° C. overnight under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide 2-(benzylamino)-4-1.5 methoxycyclohexan-1-ol as a yellow oil (1.56 g, 85.6%). LCMS (ESI) m/z 236.2, [M+H]⁺.

### Step 4: 2-amino-4-methoxycyclohexan-1-ol

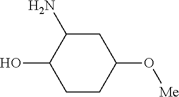

To a solution of 2-(benzylamino)-4-methoxycyclohexan-1-ol (1.20 g; 5.099 mmol; 1.00 eq.) in MeOH (35 mL) was added 10% Pd/C (600.0 mg; 50% w/w) under nitrogen atmosphere. The mixture was hydrogenated at room temperature overnight under hydrogen atmosphere. The desired product was detected via LCMS. The mixture was filtered through a celite pad, and the filtrate was concentrated under reduced pressure to provide 2-amino-4-methoxycyclohexan-1-ol as a colorless oil (394.6 mg, 53.2%). LCMS (ESI) m/z 146.1, [M+H]⁺.

### Step 5: 6-(cyclopropanecarboxamido)-IV-(2-hydroxy-5-methoxycyclohexyl)-1-(methylamino)-2,7-naphthyridine-4-carboxamide

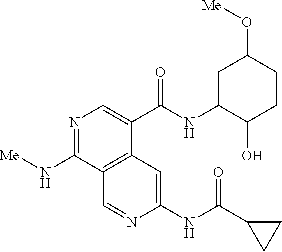 [SMILES: CNc1ncc(C(=O)NC2CC(OC)CCC2O)c2cc(NC(=O)C3CC3)ncc12]

A solution of 6-cyclopropaneamido-1-(methylamino)-2,7-naphthyridine-4-carboxylic acid (120.0 mg; 0.419 mmol; 1.00 eq.), 2-amino-4-methoxycyclohexan-1-ol (79.1 mg; 0.545 mmol; 1.30 eq.), EDCI.HCl (97.3 mg; 0.507 mmol; 1.21 eq.) and DMAP (76.8 mg; 0.629 mmol; 1.50 eq.) in DMA (5 mL) was stirred for 14 hours at room temperature under nitrogen atmosphere. The desired product was detected via LCMS. The resulting mixture was purified by flash chromatography on pre-packed C18 column using 20-50% of MeOH in water (10 mmol/L NH₄HCO₃) as eluent to provide 6-(cyclopropanecarboxamido)-N-(2-hydroxy-5-methoxycyclohexyl)-1-(methylamino)-2,7-naphthyridine-4-carboxamide as a yellow solid (146.0 mg, 84.2%). LCMS (ESI) m/z 414.2, [M+H]⁺.

### Step 6: 6-(cyclopropanecarboxamido)-N-(5-methoxy-2-oxocyclohexyl)-1-(methylamino)-2,7-naphthyridine-4-carboxamide

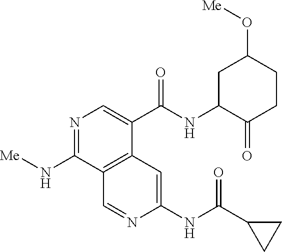 [SMILES: CNc1ncc(C(=O)NC2CC(OC)CCC2=O)c2cc(NC(=O)C3CC3)ncc12]

A solution of 6-cyclopropaneamido-N-(2-hydroxy-5-methoxycyclohexyl)-1-(methylamino)-2,7-naphthyridine-4-carboxamide (135.0 mg; 0.326 mmol; 1.00 eq.), NaHCO₃(54.8 mg; 0.652 mmol; 2.00 eq.) and Dess-Martin. (276.9 mg; 0.653 mmol; 2.00 eq.) in a mixture solvent of DMF/CH₂Cl₂ (1:7.5, 17 mL) was stirred overnight at room temperature under nitrogen atmosphere. The desired product was detected via LCMS. The resulting mixture was added to saturated NaHCO₃ solution (20 mL) and stirred at room temperature for 0.5 h. The mixture was filtered. The filtrate was diluted with CH₂Cl₂ (50 mL), washed with brine (2×10 mL), dried over Na₂SO₄. After filtration, the filtrate was concentrated under reduced pressure. The residue was purified by flash chromatography on silica gel column using 10-30% of EtOAc in petroleum ether as eluent to provide 6-(cyclopropanecarboxamido)-N-(5-methoxy-2-oxocyclohexyl)-1-(methylamino)-2,7-naphthyridine-4-carboxamide as a light-yellow solid (98.9 mg, 73.6%). LCMS (ESI) m/z 412.2, [M+H]⁺.

### Step 7: N-(5-(5-methoxy-4,5,6,7-tetrahydrobenzo[d]oxazol-2-yl)-8-(methylamino)-2,7-naphthyridin-3-yl)cyclopropanecarboxamide

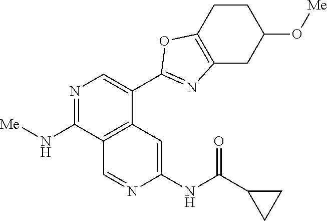 [SMILES: CNc1ncc(-c2nc3c(o2)CCC(OC)C3)c2cc(NC(=O)C3CC3)ncc12]

A solution of 6-(cyclopropanecarboxamido)-N-(5-methoxy-2-oxocyclohexyl)-1-(methylamino)-2,7-naphthyridine-4-carboxamide (40.0 mg; 0.097 mmol; 1.00 eq.) and P₂O₅ (42.0 mg; 0.296 mmol; 3.04 eq.) in NMP (4 ml) was stirred at 110° C. for 7 hours under nitrogen atmosphere. The desired product was detected via LCMS. The mixture was allowed to cool down to room temperature. The mixture was purified by flash chromatography on pre-packed C18 column using 50-70% of MeCN in water (10 mmol/L NH₄HCO₃) as eluent to afford the crude product. The product was purified by flash chromatography on silica gel column using 0-10% of MeOH in CH₂Cl₂ as eluent to provide N-(5-(5-methoxy-4,5,6,7-tetrahydrobenzo[d]oxazol-2-yl)-8-(methylamino)-2,7-naphthyridin-3-yl)cyclopropanecarboxamide as a light yellow solid (6.6 mg, 17.2%). LCMS (ESI) m/z 394.2, [M+H]⁺. ¹H NMR (400 MHz, DMSO-d₆) δ 1.0.96 (s, 1H), 9.39 (s, 1H), 9.38 (s, 1H), 8.57 (s, 1H), 8.40-8.35 (m, 1H), 3.80-3.73 (m, 1H), 3.33 (s, 3H), 3.04 (d, 0.1=4.4 Hz, 3H), 2.90-2.81 (m, 1H), 1), 2.75-2.66 (m, 2H), 2.60-2.53 (m, 1H), 2.11-1.95 (m, 3H), 0.92-0.78 (m, 4H).

### Example 875: Synthesis of N-(5-(5-methoxypyrazolo[1,5-a]pyridin-2-yl)-8-(methylamino)-2,7-naphthyridin-3-yl)cyclopropanecarboxamide

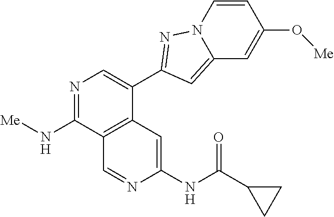 [SMILES: CNc1ncc(-c2cc3cc(OC)ccn3n2)c2cc(NC(=O)C3CC3)ncc12]

### Step 1: ethyl 2-(4-methoxypyridin-2-yl)acetate

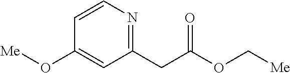 [SMILES: CCOC(=O)Cc1cc(OC)ccn1]

To a stirred solution of 4-methoxy-2-methylpyridine (5.11 g; 40.592 mmol; 1.00 eq.) and diethyl carbonate (24.21 g; 203.162 mmol; 5.00 eq.) in THF (50 mL) was added LDA (2 M in THF) (70 mL; 140.001 mmol; 3.50 eq.) dropwise at −78° C. under nitrogen atmosphere. The resulting solution was stirred at −78° C. for 2 hours. The desired product was detected via LCMS. The reaction was quenched with sat. NH₄Cl solution at 0° C. The resulting mixture was extracted with EtOAc (3×20 mL). The combined organic layers were washed with brine (3×20 mL) and dried over anhydrous Na₂SO₄. After filtration, the filtrate was concentrated under reduced pressure and purified by flash chromatography on silica gel column using 10-30% of EtOAc in petroleum ether as eluent to provide ethyl 2-(4-methoxypyridin-2-yl)acetate as a yellow oil (7.20 g, 90.8%). LCMS (ESI) m/z 196.1, [M+H]⁺.

### Step 2: 5-methoxypyrazolo[1,5-a]pyridin-2-ol

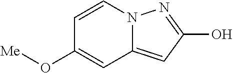 [SMILES: COc1ccn2nc(O)cc2c1]

To a stirred solution of ethyl 2-(4-methoxypyridin-2-yl)acetate (1.01 g; 5.122 mmol; 1.00 eq.) in CH₂Cl₂ (10 mL) was added O-(mesitylsulfonyl)hydroxylamine (4.40 g; 20.482 mmol; 4.00 eq.) dropwise at 0° C. The resulting mixture was stirred at room temperature overnight under nitrogen atmosphere. The desired product was detected via LCMS. The resulting mixture was concentrated under reduced pressure. The residue was purified by flash chromatography on silica gel column using 3-5% of MeOH in CH₂Cl₂ as eluent to provide 5-methoxypyrazolo[1,5-a]pyridin-2-ol as a white solid (500 mg, 59.4%). LCMS (ESI) m/z 165.1, [M+H]⁺.

### Step 3: 5-methoxypyrazolo[1,5-a]pyridin-2-yl trifluoromethanesulfonate

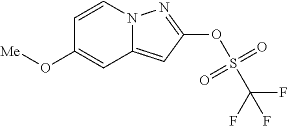 [SMILES: COc1ccn2nc(OS(=O)(=O)C(F)(F)F)cc2c1]

To a stirred solution of 5-methoxypyrazolo[1,5-a]pyridin-2-ol (200.1 mg; 1.212 mmol; 1.00 eq.) in a mixture solvent of THF/DMF (1:1, 2 mL) was added NaH (60% dispersion in mineral oil) (121.8 mg; 3.042 mmol; 2.40 eq.) at 0° C. under nitrogen atmosphere. After stirring for 10 minutes to the above solution was added 1,1,1-trifluoro-N-phenyl-N-trifluoromethanesulfonylmethanesulfonamide (522.2 mg; 1.462 mmol; 1.20 eq.) at 0° C. The resulting mixture was stirred at room temperature for 1 hour. The desired product was detected via LCMS. The reaction was quenched with sat. NH₄Cl solution (10 mL) and extracted with EtOAc (3×20 mL). The combined organic layers were washed with brine (3×10 mL) and dried over anhydrous Na₂SO₄. After filtration, the filtrate was concentrated under reduced pressure and purified by flash chromatography on silica gel column using 20-30% of EtOAc in petroleum ether as eluent to provide 5-methoxypyrazolo[1,5-a]pyridin-2-yl trifluoromethanesulfonate as a white solid (220.3 mg, 60.9%). LCMS (ER) m/z 297.0, [M+H]⁺.

### Step 4: N-(5-(5-methoxypyrazolo[1,5-a]pyridin-2-yl)-8-(methylamino)-2,7-naphthyridin-3-yl)cyclopropanecarboxamide

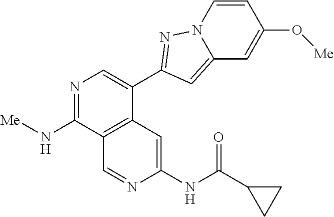 [SMILES: CNc1ncc(-c2cc3cc(OC)ccn3n2)c2cc(NC(=O)C3CC3)ncc12]

To a stirred solution of 5-methoxypyrazolo[1,5-a]pyridin-2-yl trifluoromethanesulfonate (100.2 mg; 0.332 mmol; 1.00 eq.) and N-[8-(methylamino)-5-(4,4,5,5-tetramethyl-1,3,2-dioxaborolan-2-yl)-2,7-naphthyridin-3-yl]cyclopropanecarboxamide (186.5 mg; 0.504 mmol; 1.70 eq.) in a mixture solvent of 1,4-dioxane/water (5:1, 1 mL) were added XPhos Pd G₃ (28.2 mg; 0.032 mmol; 0.01 eq.), XPhos (25.6 mg; 0.061 mmol; 0.02 eq.) and K₃PO₄ (21.5.5 mg; 1.014 mmol; 3.00 eq.) under nitrogen atmosphere. The resulting mixture was stirred at 90° C. for 1 hour. The desired product was detected via LCMS. The mixture was allowed to cool down to room temperature. The mixture was concentrated under reduced pressure and purified by flash chromatography on silica gel column using 5-8% of MeOH in CH₂Cl₂ as eluent to provide the crude product. The crude product was purified by flash chromatography on pre-packed C18 column using 30-70% of MeCN in water (10 mmol/L NH₄HCO₃) to provide N-(5-(5-methoxypyrazolo[1,5-α]pyridin-2-yl)-8-(methylamino)-2,7-naphthyridin-3-yl)cyclopropanecarboxamide as a off-white solid (28.4 mg, 21.6%). LCMS (ESI) m/z 389.2, [M+H]⁺. ¹H NMR. (400 MHz, DMSO-d₆) δ 10.94 (s, 1H), 9.39 (s, 1H), 9.00 (s, 1H), 8.54 (d, J=7.6 Hz, 1H), 8.32 (s, 1H), 8.14-8.04 (m, 1H), 7.06 (d, J=2.7 Hz, 1H), 6.62 (s, 1H), 6.59-6.52 (m, 1H), 3.86 (s, 3H), 3.03 (d, 4.4 Hz, 3H), 2.10-2.00 (m, 1H), 0.85-0.78 (m, 4H).

### Example 876: Synthesis of N-(8-(ethylamino)-5-(6-morpholinopyrazolo[1,5-a]pyridin-2-yl)-2,7-naphthyridin-3-yl)cyclopropanecarboxamide

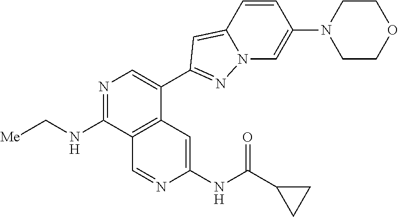 [SMILES: CCNc1ncc(-c2cc3ccc(N4CCOCC4)cn3n2)c2cc(NC(=O)C3CC3)ncc12]

### Step 1: 6-morpholinopyrazolo[1,5-a]pyridin-2-yl trifluoromethanesulfonate

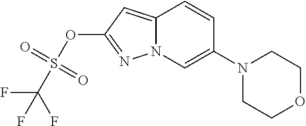 [SMILES: O=S(=O)(Oc1cc2ccc(N3CCOCC3)cn2n1)C(F)(F)F]

To a stirred solution of 6-(morpholin-4-yl)pyrazolo[1,5-c]pyridin-2-ol (Example 796, step 4) (60.4 mg; 0.272 mmol; 1.00 eq.) and DIPEA (106.1 mg; 0.812 mol; 2.50 eq.) in CH₂Cl₂ (2 nit) was added Tf₂O (115.8 mg; 0.411 mmol; 1.50 eq.) dropwise at 0° C. The resulting solution was stirred at room temperature for 1 hour under nitrogen atmosphere. The desired product was detected via LCMS. The mixture was diluted with CH₂Cl₂ (50 mL) and washed with saturated brine (3×5 nit), The organic layer was dried over Na₂SO₄, filtered and the solvent was removed under reduced pressure to afford 6-(morpholin-4-yl)pyrazolo[1,5-a]pyridin-2-yl trifluoromethanesulfonate as a green oil (80.3 mg, 83.2%). LCMS (ESI) m/z 352.1, [M+H]⁺.

### Step 2: N-(8-(ethylamino)-5-(6-morpholinopyrazolo[1,5-a]pyridin-2-yl)-2,7-naphthyridin-3-yl)cyclopropanecarboxamide

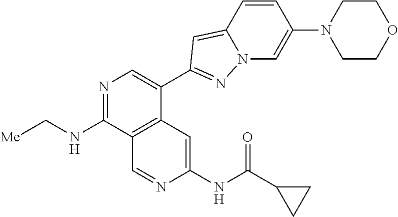 [SMILES: CCNc1ncc(-c2cc3ccc(N4CCOCC4)cn3n2)c2cc(NC(=O)C3CC3)ncc12]

To a stirred solution of 6-morpholin-4-yl)pyrazolo[1,5-a]pyridin-2-yl trifluoromethanesulfonate (80.3 mg; 0.221 mmol; 1.00 eq.) and N-(8-(ethylamino)-5-(4,4,5,5-tetramethyl-1,3,2-dioxaborolan-2-0)-2,7-naphthyridin-3-yl)cyclopropanecarboxamide (92.8 mg; 0.243 mmol; 1.10 eq.) in a mixture solvent of 1,4-dioxane/water (5:1, 1.8 mL) were added XPhos Pd C13 (19.2 mg; 0.023 mmol; 0.10 eq.), XPhos (21.7 mg; 0.042 mmol; 0.20 eq.) and K₃PO₄ (145.5 mg; 0.684 mmol, 3.00 eq.). The resulting mixture was stirred at 110° C. for 1 hour under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide the crude product. The crude product was purified by flash chromatography on pre-packed C18 column using 20-60% of MeCN in water (10 mmol/L NH₄HCO₃) as eluent to provide N-(8-(ethylamino)-5-(6-morpholinopyrazolo[1,5-a]pyridin-2-yl)-2,7-naphthyridin-3-yl)cyclopropanecarboxamide as a yellow solid (16.6 mg, 15.9%). LCMS (ESI) m/z 458.2, [M+H]⁺. ¹H NMR (400 MHz, DMSO-d₆) δ 10.95 (s, 1H), 9.43 (s, 1H), 8.86 (s, 1H), 8.26 (s, 1H), 8.14-8.09 (m, 1H), 8.00-7.95 (m, 1H), 7.62 (d, J=9.6 Hz, 1H), 7.28 (dd, 9.6, 1.6 Hz, 1H), 6.69 (s, 1H), 3.81-3.75 (m, 4H), 3.64-3.52 (m, 2H), 3.14-3.05 (m, 4H), 2.09-2.01 (m, 1H), 1.27 (t, J=7.2 Hz, 3H), 0.87-0.77 (m, 4H).

### Example 877: Synthesis of (1S,2R)—N-(5-(6-methoxypyrazolo[1,5-a]pyridin-2-yl)-8-(methylamino)-2,7-naphthyridin-3-yl)-2-methylcyclopropane-1-carboxamide

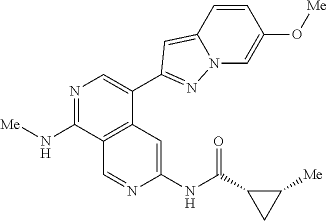 [SMILES: CNc1ncc(-c2cc3ccc(OC)cn3n2)c2cc(NC(=O)[C@H]3C[C@H]3C)ncc12]

### Step 1: 4-(6-methoxypyrazolo[1,5-a]pyridin-2-yl)-NE-methyl-2,7-naphthyridine-1,6-diamine

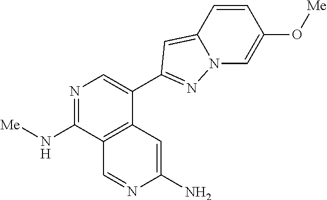 [SMILES: CNc1ncc(-c2cc3ccc(OC)cn3n2)c2cc(N)ncc12]

To a stirred solution of N-(5-(6-methoxypyrazolo[1,5-a]pyridin-2-yl)-8-(methylamino)-2,7-naphthyridin-3-yl)cyclopropanecarboxamide (Example 765) (110.2 mg; 0.284 mmol; 1.00 eq.) in a mixture solvent of DMSO/MeOH (1:1, 5 mL) was added a solution of NaOH (110.3 mg; 2.754 mmol; 1.0.00 eq.) in water. (2 mL). The resulting mixture was stirred at 80° C. for 14 hours. The desired product was detected via LCMS. The mixture was allowed to cool down to room temperature. The precipitated solids were collected by filtration and washed with MeOH (2×5 mL) to afford 4-(6-methoxypyrazolo[1,5-a]pyridin-2-yl)-N¹-methyl-2,7-naphthyridine-1,6-diamine as a yellow solid (70.2 mg, 77.1%). LCMS (ESI) m/z 321.1, [M+H]⁺.

### Step 2: (1S,2R)—N-(5-(6-methoxypyrazolo[1,5-a]pyridin-2-yl)-8-(methylamino)-2,7-naphthyridin-3-371)-2-methylcyclopropane-1-carboxamide

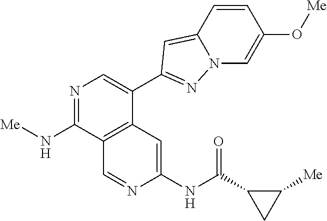 [SMILES: CNc1ncc(-c2cc3ccc(OC)cn3n2)c2cc(NC(=O)[C@H]3C[C@H]3C)ncc12]

To a stirred solution of 4-(6-methoxypyrazolo[1,5-a]pyridin-2-yl)-N¹-methyl-2,7-naphthyridine-1,6-diamine (65.2 mg; 0.204 mmol; 1.00 eq.) and (1S,2R)-2-methylcyclopropane-1-carboxylic acid (20.3 mg; 0.201 mmol; 1.00 eq.) in pyridine (2 mL) was added POCl₃ (92.6 mg; 0.602 mmol; 3.00 eq.) in portions at 0° C. under nitrogen atmosphere. The resulting solution was stirred at room temperature for 0.5 h. The desired product was detected via LCMS. The resulting mixture was diluted with EtOAc (5 mL) and quenched with water (5 mL). The resulting mixture was extracted with EtOAc (3×5 mL). The combined organic layers were washed with brine (3×20 mL) and dried over anhydrous Na₂SO₄. After filtration, the filtrate was concentrated under reduced pressure. The residue was purified by flash chromatography on silica gel column using 5-8% of MeOH: in CH₂Cl₂ as eluent to provide (1S,2R)—N-(5-(6-methoxypyrazolo[1,5-a]pyridin-2-yl)-8-(methylamino)-2,7-naphthyridin-3-yl)-2-methylcyclopropane-1-carboxamide as a yellow solid (28.3 mg, 34.6%). LCMS (ESI) m/z 403.2, [M+H]⁺. ¹H NMR (400 MHz, DMSO-d₆) δ 10.82 (s, 1H), 9.38 (s, 1H), 8.88 (s, 1H), 8.43 (d, J=2.1 Hz, 1H), 8.29 (s, 1H), 8.08-8.01 (m, 1H), 7.67 (d, J=9.6 Hz, 1H), 7.10-7.05 (m, 1H), 6.76 (s, 1H), 3.85 (s, 3H), 3.03 (d, J 4.4 Hz, 3H), 2.10-2.02 (m, 1H), 1.32-1.23 (m, 1H), 1.10 (d, J=6.1 Hz, 3H), 1.01-0.92 (m, 1H), 0.84-0.76 (m, 1H).

### Example 878: Synthesis of (1S,2R)—N-(5-(6-methoxyimidazo[1,2-a]pyridin-3-yl)-8-(methylamino)-2,7-naphthyridin-3-yl)-2-methylcyclopropane-1-carboxamide

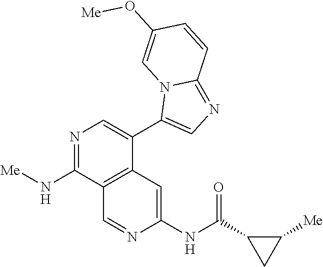 [SMILES: CNc1ncc(-c2cnc3ccc(OC)cn23)c2cc(NC(=O)[C@H]3C[C@H]3C)ncc12]

### Step 1: N-(5-(6-methoxyimidazo[1,2-a]pyridin-3-yl)-8-(methylamino)-2,7-naphthyridin-3-yl)cyclopropanecarboxamide

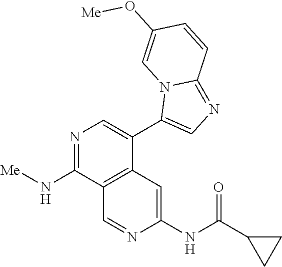 [SMILES: CNc1ncc(-c2cnc3ccc(OC)cn23)c2cc(NC(=O)C3CC3)ncc12]

To a solution of N-(8-(methylamino)-5-(4,4,5,5-tetramethyl-1,3,2-dioxaborolan-2-yl)-2,7-naphthyridin-3-yl)cyclopropanecarboxamide (200.0 mg; 0.543 mmol; 1.00 eq.) and 3-bromo-6-methoxyimidazo[1,2-a]pyridine (122.1 mg; 0.538 mmol; 0.99 eq.) in a mixture solvent of 1,4-dioxane/water (5:1, 12 mL) were added XPhos (77.6 mg; 0.163 mmol; 0.30 eq.), XPhos Pd G₃ (137.9 mg; 0.163 mmol; 0.30 eq.) and K₃PO₄ (345.8 mg; 1.629 mmol; 3.00 eq.) under nitrogen atmosphere. The reaction was stirred at 110° C. for 1 hour. The resulting mixture was concentrated under reduced pressure and purified by flash chromatography on silica gel column using 5-10% of MeOH in CH₂Cl₂ as eluent to provide N-(5-(6-methoxyimidazo[1,2-a]pyridin-3-yl)-8-(methylamino)-2,7-naphthyridin-3-yl)cyclopropanecarboxamide as a yellow solid (139 mg, 65.9%). LCMS (ER) m/z 389.2, [M+H]⁺.

### Step 2: 4-(6-methoxyimidazo[1,2-a]pyridin-3-yl)-N¹-methyl-2,7-naphthyridine-1,6-diamine

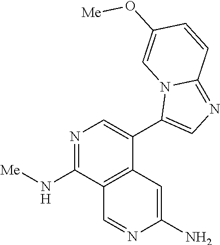 [SMILES: CNc1ncc(-c2cnc3ccc(OC)cn23)c2cc(N)ncc12]

To a solution of N-(5-(6-methoxyimidazo[1,2-a]pyridin-2-yl)-8-(methylamino)-2,7-naphthyridin-3-yl)cyclopropanecarboxamide (70.0 mg; 0.180 mmol; 1.00 eq.) in a mixture solvent of DMSO/MeOH (5:1, 6 mL) was added a solution of NaOH (70.0 mg; 1.750 mmol; 9.71 eq.) in water (2 mL). This reaction was stirred at 60° C. for 12 hours. The desired product was detected via LCMS. This mixture was concentrated under vacuum to remove MeOH. The remaining solution was purified by flash chromatography on pre-packed C18 column using 40-70% of MeCN in water (10 mmol/L NH₄HCO₃) as eluent to provide 4-(6-methoxyimidazo[1,2-a]pyridin-3-yl)-.N¹-methyl-2,7-naphthyridine-1,6-diamine as a yellow solid (50 mg, 86.6%). LCMS (ESI) m/z 321.1, [M+H]⁺.

### Step 3: (1S,2R)—N-(5-(6-methoxyimidazo[1,2-a]pyridin-3-yl)-8-(methylamino)-2,7-naphthyridin-3-yl)-2-methylcyclopropane-1-carboxamide

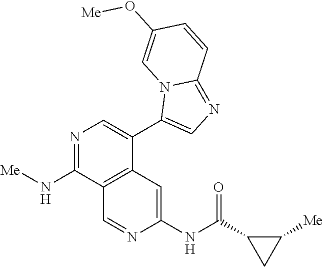 [SMILES: CNc1ncc(-c2cnc3ccc(OC)cn23)c2cc(NC(=O)[C@H]3C[C@H]3C)ncc12]

A solution of 4-(6-methoxyimidazo[1,2-a]pyridin-3-yl)-N¹-methyl-2,7-naphthyridine-1,6-diamine (50.0 mg; 0.156 mmol; 1.00 eq.) and (1S,2R)-2-methylcyclopropane-1-carboxylic acid (25.0 mg; 0.250 mmol; 1.60 eq.) in pyridine (3 mL). To the above solution was added POCl₃ (65.0 mg; 0.424 mmol; 2.72 eq.) at 0° C. under nitrogen atmosphere. The resulting solution was stirred at room temperature for 1 hour. The desired product was detected via LCMS. The reaction was diluted with EtOAc (30 mL) and washed by brine (2×5 mL). The organic layer was dried over anhydrous sodium sulfate and concentrated under vacuum. The residue was purified by flash chromatography on silica gel column using 5-10% of MeOH in CH₂Cl₂ as eluent to provide (1S,2R)—N-(5-(6-methoxyimidazo[1,2-a]pyridin-3-yl)-8-(methylamino)-2,7-naphthyridin-3-yl)-2-methylcyclopropane-1-carboxamide as a light yellow solid (26.4 mg, 42.1%). LCMS (ESI) m/z 403.2, [M+H]⁺.

### Example 879: Synthesis of N-(5-(6-methoxyimidazo[1,2-a]pyridin-2-yl)-8-(methylamino)-2,7-naphthyridin-3-yl)cyclopropanecarboxamide

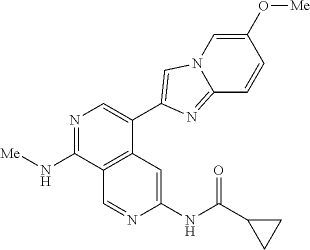 [SMILES: CNc1ncc(-c2cn3cc(OC)ccc3n2)c2cc(NC(=O)C3CC3)ncc12]

### Step 1: 6-methoxyimidazo[1,2-a]pyridin-2-ol

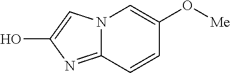 [SMILES: COc1ccc2nc(O)cn2c1]

A solution of 5-methoxypyridin-2-amine (500.0 mg; 4.028 mmol; 1.00 eq.) and ethyl 2-bromoacetate (2354.1 mg; 14.096 mmol; 3.50 eq.) in EtOH (15 mL) was stirred at 80° C. for 18 hours under nitrogen atmosphere. The desired product was detected via LCMS. The mixture was allowed to cool down to room temperature. The precipitated solids were collected by filtration, washed with Et₂O (3×5 mL) and dried under vacuum to afford 6-methoxyimidazo[1,2-a]pyridin-2-ol as an off-white solid (440.0 mg, 66.5%). LCMS (ESI) m/z 165.1, [M+H]⁺.

### Step 2: 6-methoxyimidazo[1,2-a]pyridin-2-yl trifluoromethanesulfonate

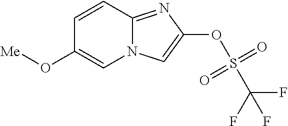 [SMILES: COc1ccc2nc(OS(=O)(=O)C(F)(F)F)cn2c1]

To a solution of 6-methoxyimidazo[1,2-a]pyridin-2-ol (420.0 mg; 2.558 mmol; 1.00 eq.) in a mixture solvent of DMF/THF (1:1, 8 mL) was added NaH (60% dispersion in mineral oil) (113.5 mg; 4.730 mmol; 1.85 eq.) at 0° C. under nitrogen atmosphere. After stirring for 10 minutes, to the above solution was added 1,1,1-trifluoro-N-phenyl-N-((trifluoromethyl)sulfonyl)methanesulfonamide (1096.7 mg; 3.070 mmol; 1.20 eq.) in portions at room temperature. The resulting mixture was stirred at room temperature for 2 hours. The desired product was detected via LCMS. The reaction was quenched by the addition of ice water (10 mL) at 0° C. The resulting mixture was extracted with EtOAc (3×10 mL). The combined organic layers were washed with brine (3×10 mL), dried over anhydrous Na₂SO₄. After filtration, the filtrate was concentrated under reduced pressure and purified by flash chromatography on silica gel column using 30-60% of EtOAc in petroleum ether as eluent to provide 6-methoxyimidazo[1,2-a]pyridin-2-yl trifluoromethanesulfonate (410.0 mg, 54.1%) as a yellow solid. LCMS (ESI) m/z 297.0, [M+H]⁺.

### Step 3: N-(5-(6-methoxyimidazo[1,2-a]pyridin-2-yl)-8-(methylamino)-2,7-naphthyridin-3-yl)cyclopropanecarboxamide

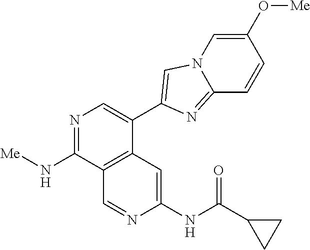 [SMILES: CNc1ncc(-c2cn3cc(OC)ccc3n2)c2cc(NC(=O)C3CC3)ncc12]

To a solution of 6-methoxyimidazo[1,2-a]pyridin-2-yl trifluoromethanesulfonate (1.00.0 mg; 0.338 mmol; 1.00 eq.) and N-(8-(methylamino)-5-(4,4,5,5-tetramethyl-1,3,2-dioxaborolan-2-yl)-2,7-naphthyridin-3-yl)cyclopropanecarboxamide (251.0 mg; 0.682 mmol; 2.02 eq.) in a mixture solvent of 1,4-dioxane/water (5:1, 6 mL) was added XPhos Pd G₃ (28.6 mg; 0.034 mmol; 0.10 eq.), XPhos (32.2 mg; 0.068 mmol; 0.20 eq.) and K₃PO₄ (143.3 mg; 0.675 mmol; 2.00 eq.) under nitrogen atmosphere. The resulting mixture was stirred at 110° C. for 2 hours. The desired product was detected via LCMS. The mixture was allowed to cool down to room temperature. The residue was purified by flash chromatography on silica gel column using 1-6% of MeOH in CH₂Cl₂ as eluent to provide the crude product. The crude product was repurified by flash chromatography on pre-packed C18 column using 50-90% of MeOH in water (10 mmol/L NH₄HCO₃) as eluent to provide N-(5-(6-methoxyimidazo[1,2-a]pyridin-2-yl)-8-(methylamino)-2,7-naphthyridin-3-yl)cyclopropanecarboxamide as a yellow solid (28.9 mg, 22.1%). LCMS (ESI) m/z 389.2, [M+H]⁺. ¹H NMR (400 MHz, DMSO-d₆) δ 10.92 (s, 1H), 9.38 (s, 1H), 8.92 (s, 1H), 8.31 (s, 1H), 8.30 (s, 1H), 8.05-8.00 (m, 2H), 7.52 (d, J=9.8 Hz, 1H), 7.05 (dd, J=9.8, 2.4 Hz, 1H), 3.83 (s, 3H), 3.03 (d, J=4.2 Hz, 3H), 2.11-2.01 (m, 1H), 0.88-0.77 (m, 4H).

### Example 880: Synthesis of N-(8-(methylamino)-5-(6-morpholinoimidazo[1,2-a]pyridin-2-yl)-2,7-naphthyridin-3-yl)cyclopropanecarboxamide

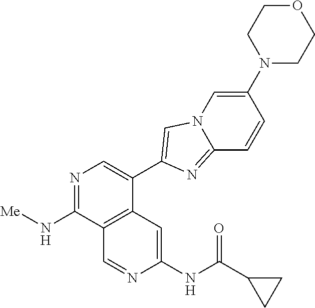 [SMILES: CNc1ncc(-c2cn3cc(N4CCOCC4)ccc3n2)c2cc(NC(=O)C3CC3)ncc12]

### Step 1: 4-(2-chloroimidazo[1,2-a]pyridin-6-yl)morpholine

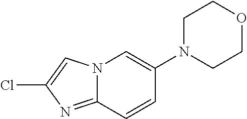 [SMILES: Clc1cn2cc(N3CCOCC3)ccc2n1]

A mixture of 6-bromo-2-chloroimidazo[1,2-a]pyridine (150.0 mg; 0.648 mmol; 1.00 eq.), morpholine (56.4 mg; 0.648 mmol; 1.00 eq.), Pd-PEPPSI-IHeptCl (63.1 mg; 0.065 mmol; 0.10 eq.) and Cs₂CO₃ (844.5 mg; 2.592 mmol; 4.00 eq.) in 1,4-dioxane (10 mL) was stirred at 100° C. overnight under nitrogen atmosphere. The desired product was detected via LCMS. The resulting mixture was concentrated under reduced pressure and purified by flash chromatography on silica gel column using 0-20% of EtOAc in petroleum ether as eluent to provide 4-(2-chloroimidazo[1,2-a]pyridin-6-yl)morpholine as an off-white solid (48.1 mg, 31.2%). LCMS (ESI) m/z 238.1, [M+H]⁺.

### Step 2: N-(8-(methylamino)-5-(6-morpholinoimidazo[1,2-a]pyridin-2-yl)-2,7-naphthyridin-3-yl)cyclopropanecarboxamide

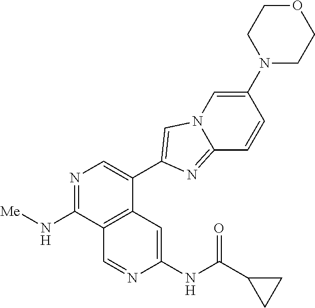 [SMILES: CNc1ncc(-c2cn3cc(N4CCOCC4)ccc3n2)c2cc(NC(=O)C3CC3)ncc12]

A mixture of N-(8-(methylamino)-5-(4,4,5,5-tetramethyl-1,3,2-dioxaborolan-2-yl)-2,7-naphthyridin-3-yl)cyclopropanecarboxamide (63.2 mg; 0.172 mmol; 1.00 eq.), XPhos Pd G₃ (43.5 mg; 0.051 mmol; 0.30 eq.), XPhos (24.5 mg; 0.052 mmol; 0.30 eq.) and K₃PO₄ (109.3 mg; 0.515 mmol; 3.00 eq.) in a mixture solvent of 1,4-dioxane/water (5:1, 2.5 mL) was stirred at 1.1.0° C. for 1 hour under nitrogen atmosphere. The desired product was detected via LCMS. The resulting mixture was concentrated under reduced pressure. The residue was purified by flash chromatography on silica gel column using 0-10% of MeOH in CH₂Cl₂ as eluent to provide N-(8-(methylamino)-5-(6-morpholinoimidazo[1,2-a]pyridin-2-yl)-2,7-naphthyridin-3-yl)cyclopropanecarboxamide as a off-white solid (15.5 mg, 20.3%). LCMS (ESI) m/z 444.2, [M+H]⁺. ¹H NMR (400 MHz, DMSO-d₆) δ 10.92 (s, 1H), 9.38 (s, 1H), 8.91 (s, 1H), 8.30 (s, 1H), 8.05 (d, J=2.0 Hz, 1H), 8.03-7.98 (m, 1H), 7.97 (s, 1H), 7.49 (d, J=9.6 Hz, 1H), 7.26 (dd, J=9.6, 2.0 Hz, 1H), 3.84-3.72 (m, 4H), 3.11-3.03 (m, 4H), 3.02 (d, J=4.4 Hz, 3H), 2.10-2.00 (m, 1H), 0.85-0.77 (m, 4H).

### Example 881: Synthesis of N-(8-(methylamino)-5-(5-phenyl-1H-pyrazol-3-yl)-2,7-naphthyridin-3-yl)cyclopropanecarboxamide

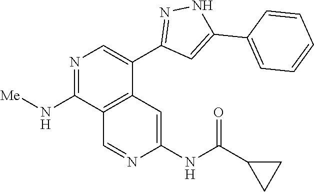 [SMILES: CNc1ncc(-c2cc(-c3ccccc3)[nH]n2)c2cc(NC(=O)C3CC3)ncc12]

### Step 1: 3-bromo-5-phenyl-1-(tetrahydro-2H-pyran-2-yl)-1H-pyrazole and 5-bromo-3-phenyl-1-(tetrahydro-2/1-pyran-2-yl)-1h-pyrazole

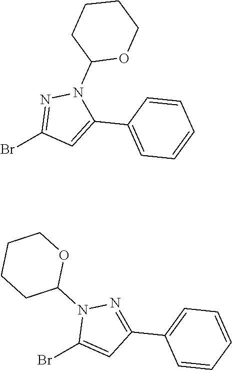 [SMILES: Brc1cc(-c2ccccc2)n(C2CCCCO2)n1 ; Brc1cc(-c2ccccc2)nn1C1CCCCO1]

To a solution of 3,5-dibromo-1-(tetrahydro-2H-pyran-2-yl)-1H pyrazole (1.00 g; 3.226 mmol; 1.00 eq.) in a mixture solvent of DME/water (5:1, 12 mL) were added Pd(PPh₃)₄ (372.4 mg; 0.322 mmol; 0.10 eq.), Na₂CO₃ (690.6 mg; 6.452 mmol; 2.00 eq.) and phenylboronic acid (398.0 mg; 3.226 mmol; 1.00 eq.). The reaction was stirred at 100° C. for 2 hours under nitrogen atmosphere. The desired product was detected via LCMS. The resulting mixture was concentrated under vacuum. The residue was purified by flash chromatography on silica gel column using 20-70% of EtOAc in petroleum ether as eluent to provide a mixture of 3-bromo-5-phenyl-1-(tetrahydro-2H-pyran-2-yl)-1H pyrazole and 5-bromo-3-phenyl-1-(tetrahydro-2H-pyran-2-yl)-1H-pyrazole as a white oil (680.0 mg, 68.5%). LCMS (ESI) m/z 307.0, [M+H]⁺.

### Step 2: N-(8-(methylamino)-5-(3-phenyl-1-(tetrahydro-2H-pyran-2-yl)-1H-pyrazol-5-yl)-2,7-naphthyridin-3-yl)cyclopropanecarboxamide and N-(8-(methylamino)-5-(5-phenyl-1-(tetrahydro-2H-pyran-2-yl)-1H-pyrazol-3-yl)-2,7-naphthyridin-3-yl) cyclopropanecarboxamide

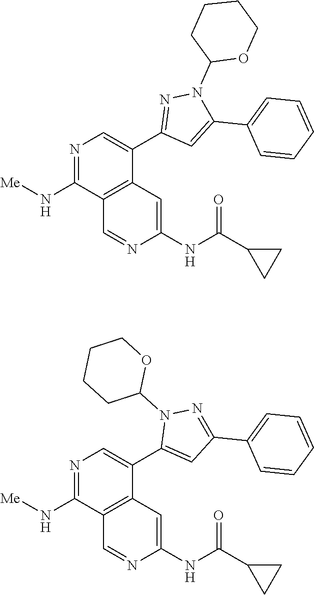 [SMILES: CNc1ncc(-c2cc(-c3ccccc3)n(C3CCCCO3)n2)c2cc(NC(=O)C3CC3)ncc12 ; CNc1ncc(-c2cc(-c3ccccc3)nn2C2CCCCO2)c2cc(NC(=O)C3CC3)ncc12]

To a mixture of 3-bromo-5-phenyl-1-(tetrahydro-2H-pyran-2-yl)-1H-pyrazole and 5-bromo-3-phenyl-1-(tetrahydro-2H-pyran-2-yl)-1H-pyrazole (250.0 mg; 0.811 mmol; 1.00 eq.) in a mixture solvent of 1,4-dioxane/water (5:1, 12 mL) was added XPhos Pd G₃ (55.0 mg; 0.065 mmol; 0.10 eq.), XPhos (30.9 mg; 0.065 mmol; 0.10 eq.), K₃PO₄ (275.6 mg; 1.593 mmol; 3.00 eq.) and N-(8-(methylamino)-5-(4,4,5,5-tetramethyl-1,3,2-dioxaborolan-2-yl)-2,7-naphthyridin-3-yl) cyclopropanecarboxamide (300.0 mg; 0.811 mmol; 1.00 eq.). The reaction was stirred at 90° C. for 1 hour under nitrogen atmosphere. The desired product was detected via LCMS. The resulting mixture was concentrated under vacuum and purified by flash chromatography on silica gel column using 2-10% of MeOH in CH₂Cl₂ as eluent to provide the crude product. The product was re-purified by flash chromatography on pre-packed C18 column using 20-80% of MeCN in water (10 mmol/L NH₄HCO₃) as eluent to provide a mixture of N-(8-(methylamino)-5-(3-phenyl-1-(tetrahydro-2H-pyran-2-yl)-1H-pyrazol-5-yl)-2,7-naphthyridin-3-yl)cyclopropanecarboxamide and N-(8-(methylamino)-5-(5-phenyl-1-(tetrahydro-2H-pyran-2-yl)-1H-pyrazol-3-yl)-2,7-naphthyridin-3-yl) cyclopropanecarboxamide as a white solid (60.0 mg, 15.7%). LCMS (ESI) m/z 469.2, [M+H]⁺.

### Step 3: N-(8-(methylamino)-5-(5-phenyl-1H-pyrazol-3-yl)-2,7-naphthyridin-3-yl) cyclopropanecarboxamide

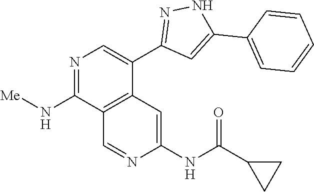 [SMILES: CNc1ncc(-c2cc(-c3ccccc3)[nH]n2)c2cc(NC(=O)C3CC3)ncc12]

To a solution of N-(8-(methylamino)-5-(3-phenyl-1-(tetrahydro-2H-pyran-2-yl)-1H-pyrazol-5-yl)-2,7-naphthyridin-3-yl)cyclopropanecarboxamide and N-(8-(methylamino)-5-(5-phenyl-1-(tetrahydro-2H-pyran-2-yl)-1H-pyrazol-3-yl)-2,7-naphthyridin-3-yl) cyclopropanecarboxamide (60.0 mg; 0.128 mmol; 1.00 eq.) in MeOH (1 mL) was added HCl (4 M in 1,4-dioxane, 5 mL). The reaction was stirred at room temperature for 3 hours under nitrogen atmosphere. The desired product was detected via LCMS. The resulting mixture was concentrated under vacuum and purified by flash chromatography on pre-packed C18 column using 20-80% of MeCN in water (10 mmol/L NH₄HCO₃) as eluent to provide N-(8-(methylamino)-5-(5-phenyl-1H-pyrazol-3-yl)-2,7-naphthyridin-3-yl) cyclopropanecarboxamide as a white solid (33.1 mg, 15.7%). LCMS (ESI) m/z 385.2, [M+H]⁺. ¹H NMR (400 MHz, DMSO-d₆) δ 13.61-12.93 (m, 1H), 11.09-10.90 (m, 1H), 9.44-9.34 (m, 1H), 9.20-8.52 (m, 1H), 8.36-7.94 (m, 2H), 7.93-7.71 (m, 2H), 7.53-7.30 (m, 3H), 7.01-6.79 (m, 1H), 3.03 (d, J=4.4 Hz, 3H), 2.10-1.99 (m, 1H), 0.95-0.64 (m, 4H).

### Example 882: Synthesis of N-(5-((1-methyl-1H-benzo[d][1,2,3]triazol-6-yl)ethynyl)-8-(methylamino)pyrido[3,4-c]pyridazin-3-yl)cyclopropanecarboxamide

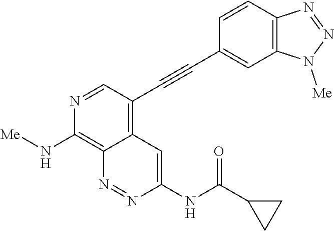 [SMILES: CNc1ncc(C#Cc2ccc3nnn(C)c3c2)c2cc(NC(=O)C3CC3)nnc12]

### Step 1: N-(8-(methylamino)pyrido[3,4-c]pyridazin-3-yl)cyclopropanecarboxamide

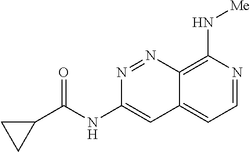 [SMILES: CNc1nccc2cc(NC(=O)C3CC3)nnc12]

To a stirred solution of N-(8-oxo-7,8-dihydropyrido[3,4-c]pyridazin-3-yl)cyclopropanecarboxamide (2.30 g; 9.990 mmol; 1 eq.) and methylamine (2 M THF, 25 mL; 5.00 eq.) in DMA (50 mL) were added DIEA (10.50 g; 81.240 mmol; 8.1.3 eq.) and PyBOP (15.70 g; 30.169 mmol; 3.02 eq.) at room temperature under nitrogen atmosphere. The resulting mixture was stirred at 100° C. for 16 hours. The desired product was detected via LCMS. The mixture was allowed to cool down to room temperature and concentrated under reduced pressure. The residue was purified by flash chromatography on pre-packed C18 column using 20-60% of MeCN in water (10 mmol/L NH₄HCO₃) to afford a crude product thatwas repurified by flash chromatography on silica gel column using 5-1.5% of MeOH in CH₂Cl₂ as eluent to provide N-(8-(methylamino)pyrido[3,4-c]pyridazin-3-yl)cyclopropanecarboxamide as a yellow solid (1.10 g, 45.2%). LCMS (ESI) m/z 244.1, [M+H]⁺.

### Step 2: N-(5-bromo-8-(methylamino)pyrido[3,4-c]pyridazin-3-yl)cyclopropanecarboxamide

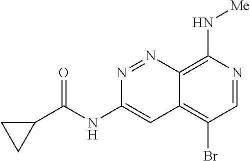 [SMILES: CNc1ncc(Br)c2cc(NC(=O)C3CC3)nnc12]

A solution of N-(8-(methylamino)pyrido[3,4-c]pyridazin-3-yl)cyclopropanecarboxamide (1.00 g; 4.111 mmol; 1 eq.) and NBS (730.0 mg; 4.1.01 mmol; 1.00 eq.) in DMF (6 mL) was stirred at room temperature for 1 hour under nitrogen atmosphere. The desired product was detected via LCMS. To the mixture was added water (30 mL) to precipitate solids. The precipitated solids were collected by filtration, washed with water (10 mL) and dried under reduced pressure to afford N-(5-bromo-8-(methylamino)pyrido[3,4-c]pyridazin-3-yl)cyclopropanecarboxamide as a yellow solid (1.00 g; 75.5%). LCMS (ESI) m/z 322.0, [M+H]⁺.

### Step 3: N-(5-((1-methyl-1H-benzo[d][1,2,3]triazol-6-yl)ethynyl)-8-(methylamino)pyrido[3,4-c]pyridazin-3-yl)cyclopropanecarboxamide

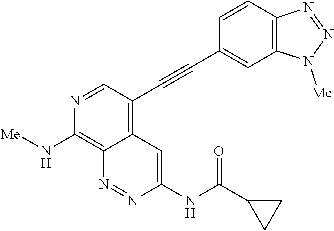 [SMILES: CNc1ncc(C#Cc2ccc3nnn(C)c3c2)c2cc(NC(=O)C3CC3)nnc12]

To a stirred solution of yl)cyclopropanecarboxamide (100.0 mg; 0.310 mmol; 1.00 eq.), XPhos Pd G₃ (26.3 mg; 0.031 mmo; 0.10 eq.) and XPhos (14.8 mg; 0.031 mmol, 0.10 eq.) in IMF (3 ml) were added Et₃N (125.6 mg; 1.240 mmol; 3.00 eq.), 6-ethynyl-1-1-methyl-1H-benzo[d][1,2,3]triazole (58.6 mg, 0.371 mmol, 1.20 eq.) and CuI (5.9 mg; 0.031 mmol; 0.10 eq.) under nitrogen atmosphere. The resulting solution was stirred at 110° C. for 6 hours. The desired product was detected via LCMS. The reaction was diluted with water (10 mL) and extracted with EtOAc (2×10 mL). The combined organic layers were washed with brine (2×10 mL), dried over anhydrous Na₂SO₁. After filtration, the filtrate was concentrated under reduced pressure. The residue was purified by flash chromatography on silica gel column using 5-10% of MeOH in CH₂Cl₂ as eluent to provide the crude product. The product was further purified by flash chromatography on pre-packed C18 column using 30-100% of MeCN in water (10 mmol/L NH₄HCO₃) to provide N-(54(1-methyl-1H-benzo[d][1,2,3]triazol-6-yl)ethynyl)-8-(methylamino)pyrido[3,4-c]pyridazin-3-yl)cyclopropanecarboxamide (26.1 mg, 21.1%) as a brown solid. LCMS:(ES, m/z): [M+H]⁺=399.2. ¹HNMR (400 MHz, DMSO-d₆) δ 11.86 (s, 1H), 9.14-9.09 (m, 1H), 8.87 (s, 1H), 8.37 (s, 1H), 8.18-8.14 (m, 2H), 7.53 (d, J=8.4 Hz, 1H), 4.35 (s, 3H), 3.10 (d, J=4.8 Hz, 3H), 2.24-2.18 (m, 1H), 0.99-0.91 (m, 4H).

### Example 883: Synthesis of (R)—N-(8-(methylamino)-5-((4-(2-methylmorpholino)phenyl)ethynyl)pyrido[3,4-c]pyridazin-3-yl)cyclopropanecarboxamide

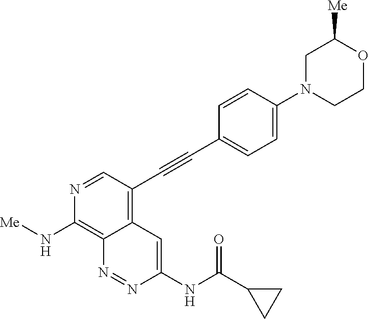 [SMILES: CNc1ncc(C#Cc2ccc(N3CCO[C@H](C)C3)cc2)c2cc(NC(=O)C3CC3)nnc12]

### Step 1: (R)-4-(4-bromophenyl)-2-methylmorpholine

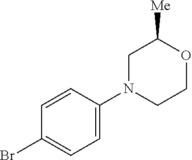 [SMILES: C[C@@H]1CN(c2ccc(Br)cc2)CCO1]

To a solution of 1,4-dibromobenzene (300.0 mg; 1.272 mmol; 1.00 eq.) in toluene (2 mL) were added (R)-2-methylmorpholine (64.0 mg, 0.633 mmol, 0.50 eq.), Pd₂(dba)₃ (58.0 mg; 0.1.01 mmol; 0.08 eq.), BINAP (79.0 mg; 0.127 mmol; 0.10 eq.), sodium t-butoxide (85.0 mg; 0.884 mmol; 0.70 eq.) under nitrogen atmosphere. The resulting mixture was stirred at 80° C. overnight. The solvent was concentrated under reduced pressure. The residue was purified by flash chromatography on silica gel column using 10-20% of EtOAc in petroleum ether as eluent to provide (R)-4-(4-bromophenyl)-2-methylmorpholine (180 mg, 55.3%) as a yellow solid. LCMS (ESI) m/z 256.0, [M+H]⁺.

### Step 2: (R)-2-methyl-4-(4-((trimethylsilyl)ethynyl)phenyl)morpholine

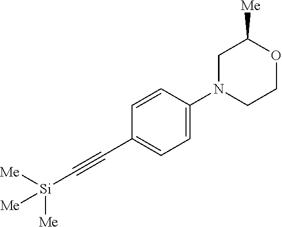 [SMILES: C[C@@H]1CN(c2ccc(C#C[Si](C)(C)C)cc2)CCO1]

To a solution of (R)-4-(4-bromophenyl)-2-methylmorpholine (160.0 mg; 0.625 mmol; 1 eq.) in DMF (2 mL) were added ethynyltrimethylsilane (203.0 mg; 2.067 mmol; 3.31 eq.), piperidine (106.7 mg; 1.253 mmol; 2.01 eq.), PPh₃ (32.9 mg; 0.125 mmol; 0.20 eq.), Pd(PPh₃)₂Cl₂ (88.1 mg; 0.126 mmol; 0.20 eq.) and CuI (11.9 mg; 0.062 mmol; 0.10 eq.). The resulting mixture was stirred at 90° C. overnight under nitrogen atmosphere. The mixture was diluted with water (10 mL) and extracted with EtOAc (3×10 mL). The combined organic layers were washed with brine (3×10 mL), dried over anhydrous Na₂SO₄. After filtration, the filtrate was concentrated under reduced pressure. The residue was purified by flash chromatography on silica gel column using 10-20% of EtOAc in petroleum ether as eluent to provide (R)-2-methyl-4-(4-((trimethylsilyl)ethynyl)phenyl)morpholine (130 mg, 76.1%) as a brown solid. LCMS (ESI) m/z 274.2, [M+H]⁺.

### Step 3: (R)-4-(4-ethynylphenyl)-2-methylmorpholine

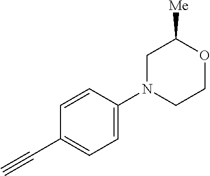

To a solution of (R)-2-methyl-4-(4-((trimethylsilyl)ethynyl)phenyl)morpholine (130.0 mg, 0.475 mmol, 1 eq.) in MeOH (3 mL) was added K₂CO₃ (197.0 mg, 1.425 mmol, 3.00 eq.). The resulting solution was stirred at room temperature for 2 hours. The resulting mixture was concentrated under reduced pressure. The residue was purified by flash chromatography on silica gel column using 10-20% of EtOAc in petroleum ether as eluent to provide (R)-4-(4-ethynylphenyl)-2-methylmorpholine (90 mg, 94.1%) as a yellow solid. LCMS (ESI) m/z 202.1, [M+H]⁺.

### Step 4: (R)—N-(8-(methylamino)-5-((4-(2-methylmorpholino)phenyl)ethynyl)pyrido[3,4-c]pyridazin-3-yl)cyclopropanecarboxamide

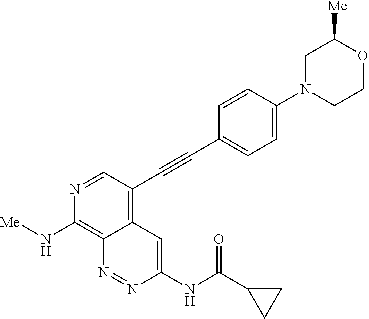 [SMILES: CNc1ncc(C#Cc2ccc(N3CCO[C@H](C)C3)cc2)c2cc(NC(=O)C3CC3)nnc12]

To a solution of (R)-4-(4-ethynylphenyl)-2-methylmorpholine (50.0 mg; 0.248 mmol; 1 eq.) and N-(5-bromo-8-(methylamino)pyrido[3,4-c]pyridazin-3-yl)cyclopropanecarboxamide (53.4 mg; 0.166 mmol; 0.67 eq.) in DMF (2 mL) were added Et₃N (67.0 mg; 0.662 mmol; 2.67 eq.), XPhos (15.8 mg; 0.033 mmol; 0.13 eq.), XPhos Pd (13 (28.1 mg; 0.033 mmol; 0.13 eq.) and CuI (3.2 mg; 0.017 mmol; 0.07 eq.) under nitrogen atmosphere. The resulting solution was stirred at 90° C. for 1 hour. The mixture was diluted with water. (10 mL) and extracted with EtOAc (3×10 mL). The combined organic layers were washed with brine (3×30 mL), dried over anhydrous Na₂SO₄. After filtration, the filtrate was concentrated under reduced pressure. The residue was purified by flash chromatography on silica gel column using 10-15% of MeOH in CH₂Cl₂ as eluent to provide (R)—N-(8-(methylamino)-5-((4-(2-methylmorpholino)phenyl)ethynyl)pyrido[3,4-c]pyridazin-3-yl)cyclopropanecarboxamide (24.1 mg, 21.9%) as a red solid. LCMS (ESI) m/z 443.2, [M+H]⁺. ¹H NMR (400 MHz, DMSO-d₆) δ 1.1.80 (s, 1H), 8.98-8.91 (m, 1H), 8.78 (s, 1H), 8.25 (s, 1H), 7.41 (d, J=8.8 Hz, 2H), 7.00 (d, =8.8 Hz, 2H), 3.97-3.87 (m, 1H), 3.75-3.69 (m, 1H), 3.67-3.58 (m, 3H), 3.08 (d, J=4.4 Hz, 3H), 2.78-2.65 (m, 1H), 2.43-2.37 (m, 1H), 2.23-2.18 m, 1H), 1.17 (d, J=6.0 Hz, 3H), 0.96-0.89 (m, 4H).

### Example 884: Synthesis of N-(5-((5-methoxypyrid in-2-yl)ethynyl)-8-(methylamino)pyrido[3,4-c]pyridazin-3-yl)cyclopropanecarboxamide

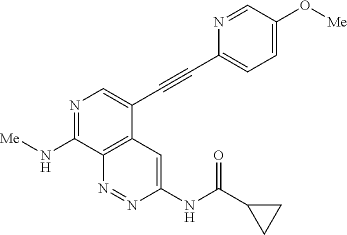 [SMILES: CNc1ncc(C#Cc2ccc(OC)cn2)c2cc(NC(=O)C3CC3)nnc12]

To a solution of N-(5-bromo-8-(methylamino)pyrido[3,4-c]pyridazin-3-yl)cyclopropanecarboxamide (60.0 mg; 0.18 mmol; 1.00 eq.) and 2-ethynyl-5-methoxypyridine (50.0 mg; 0.37 mmol; 2.02 eq.) in DMF (2 mL) were added Et₃N (33.0 mg; 0.32 mmol; 1.75 eq.), CuI (5.0 mg; 0.02 mmol; 0.14 eq.), XPhos Pd G₃ (15.0 mg; 0.01 mmol; 0.1.0 eq.) and XPhos (18.0 mg; 0.03 mmol; 0.20 eq.) at room temperature. The resulting mixture was stirred at 110° C. for 2 h under nitrogen atmosphere. The desired product was detected via LCMS. The mixture was allowed to cool down to room temperature. The resulting mixture was purified by flash chromatography on pre-packed C18 column using 20-60% of MeCN in water (10 mmol/L NH₁HCO₃) to afford N-(5-((5-methoxypyridin-2-yl)ethynyl)-8-(methylamino)pyrido[3,4-c]pyridazin: 3-yl)cyclopropanecarboxamide as a brown solid (32.6 mg, 44.5%). LCMS (ESI) m/z 375.1, [M+H]⁺. ¹H NMR (400 MHz, DMSO-d₆) δ 1.1.82 (s, 1H), 9.10-9.04 (m, 1H), 8.75 (s, 1H), 8.35 (s, 1H), 8.33 (d, J=3.2 Hz, 1H), 7.59 (d, J=8.8 Hz, 1H), 7.48 (dd, J=8.8, 3.2 Hz, 1H), 3.88 (s, 3H), 3.09 (d, J=4.4 Hz, 3H), 2.25-2.15 (m, 1H), 0.97-0.83 (m, 4H).

### Example 885: Synthesis of N-(8-(methylamino)-5-(6-morpholino-[1,2,4]triazolo[1,5-a]pyridin-2-yl)pyrido[3,4-c]pyridazin-3-yl)cyclopropanecarboxamide

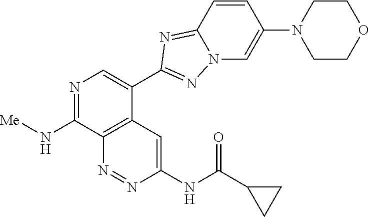 [SMILES: CNc1ncc(-c2nc3ccc(N4CCOCC4)cn3n2)c2cc(NC(=O)C3CC3)nnc12]

### Step 1: N-(8-(methylamino)-5-(4,4,5,5-tetramethyl-1,3,2-dioxaborolan-2-yl)pyrido[3,4-c]pyridazin-3-yl)cyclopropanecarboxamide

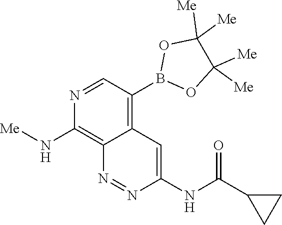 [SMILES: CNc1ncc(B2OC(C)(C)C(C)(C)O2)c2cc(NC(=O)C3CC3)nnc12]

To a stirred solution of N-(5-bromo-8-(methylamino)pyrido[3,4-c]pyridazin-3-yl)cyclopropanecarboxamide (300.0 mg; 0.931 mmol; 1.00 eq.) and 4,4,4′,4′,5,5,5′,5′-octamethyl-2,2′-bi(1,3,2-dioxaborolane) (2370.0 mg; 9.312 mmol; 10.00 eq.) in 1,4-dioxane (4 mL) were added Pd(dppf)Cl₂CH₂Cl₂ (75.8 mg; 0.093 mmol; 0.10 eq.) and KOAc (181.8 mg; 1.852 mmol; 1.99 eq.). The resulting mixture was stirred at 100° C. for 2 hours under nitrogen atmosphere. The desired product was detected via LCMS. The mixture was allowed to cool down to room temperature and concentrated under reduced pressure. The residue was dissolved with CH₂Cl₂ (25 mL), the insoluble solids were filtered out and washed with CH₂Cl₂ (3×10 mL). The filtrate was concentrated under reduced pressure. The residue was dissolved in CH₂Cl₂ (3 mL), this solution was added dropwise into n-hexane (30 mL). The precipitated solids were collected by filtration and washed with n-hexane (10 mL) to afford N-(8-(methylamino)-5-(4,4,5,5-tetramethyl-1,3,2-dioxaborolan-2-yl)pyrido[3,4-c]pyridazin-3-yl)cyclopropanecarboxamide as a yellow solid (330.0 mg, 96.0%). LCMS (ESI) m/z 370.2, [M+H]⁺.

### Step 2: N-(8-(methylamino)-5-(6-morpholino-[1,2,4]triazolo[1,5-a]pyridin-2-yl)pyrido[3,4-c]pyridazin-3-yl)cyclopropanecarboxamide

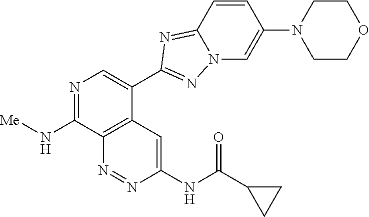 [SMILES: CNc1ncc(-c2nc3ccc(N4CCOCC4)cn3n2)c2cc(NC(=O)C3CC3)nnc12]

A suspension of N-(8-(methylamino)-5-(4,4,5,5-tetramethyl-1,3,2-dioxaborolan-2-yl)pyrido[3,4-c]pyridazin-3-yl)cyclopropanecarboxamide (240.0 mg; 0.650 mmol; 1.00 eq.), 4-(2-chloro-[1,2,4]triazolo[1,5-a]pyridin-6-yl)morpholine (69.3 mg; 0.290 mmol; 0.30 eq.), XPhos Pd G₃ (36.7 mg; 0.043 mmol; 0.07 eq.), XPhos (41.3 mg; 0.087 mmol; 0.13 eq.), K₃PO₄ (275.9 mg; 1.300 mmol; 2.00 eq.) in a mixture solvent of 1,4-dioxane/water (5:1, 2.4 mL) was stirred at 110° C. for 1 hour under nitrogen atmosphere. The desired product was detected via LCMS. The resulting mixture was concentrated under reduced pressure. The residue was purified by flash chromatography on silica gel column using 3-5% of MeOH in CH₂Cl₂ as eluent to provide N-(8-(methylamino)-5-(6-morpholino-[1,2,4]triazolo[1,5-a]pyridin-2-yl)pyrido[3,4-c]pyridazin-3-yl)cyclopropanecarboxamide as a yellow solid (89.1 mg, 44.1%). LCMS (ESI) m/z 446.2, [M+H]⁺. ¹H NMR (400 MHz, DMSO-d₆) δ 11.69 (s, 1H), 9.77 (s, 1H), 9.06-8.91 (m, 1H), 8.85 (s, 1H), 8.32 (d, J=1.2 Hz, 1H), 7.82-7.68 (m, 2H), 3.90-3.72 (m, 4H), 3.25-3.14 (m, 4H), 3.12 (d, J=4.8 Hz, 3H), 2.28-2.16 (m, 1H), 0.99-0.85 (m, 4H).

### Example 886: Synthesis of 1-fluoro-N-(8-(methylamino)-5-(6-morpholino-[1,2,4]triazolo[1,5-a]pyridin-2-yl)pyrido[3,4-c]pyridazin-3-yl)cyclopropane-1-carboxamide

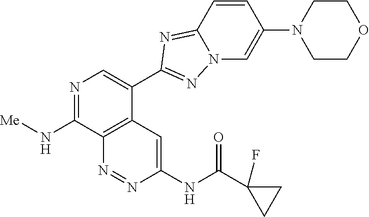 [SMILES: CNc1ncc(-c2nc3ccc(N4CCOCC4)cn3n2)c2cc(NC(=O)C3(F)CC3)nnc12]

### Step 1: N⁸-methyl-5-(6-morpholino-[1,2,4]triazolo[1,5-a]pyridin-2-yl)pyrido[3,4-c]pyridazine-3,8-diamine

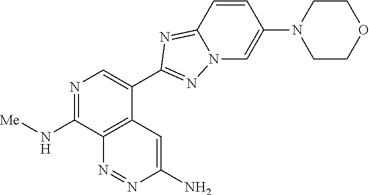 [SMILES: CNc1ncc(-c2nc3ccc(N4CCOCC4)cn3n2)c2cc(N)nnc12]

To a solution of N-(8-(methylamino)-5-(6-morpholino-[1,2,4]triazolo[1,5-a]pyridin-2-yl)pyrido[3,4-c]pyridazin-3-yl)cyclopropanecarboxamide, Example 885, (100.0 mg; 0.220 mmol; 1.00 eq.) in a mixture solvent of DMSO/MeOH (1:1, 5 mL) was added a solution of NaOH (90.0 mg; 2.250 mmol; 10.00 eq.) in water (2.5 mL) at room temperature. The resulting mixture was stirred at 60° C. for 16 hours. The desired product was detected via LCMS. The resulting mixture was concentrated under reduced pressure to remove MeOH. To the mixture was added water (10 mL) to precipitate solids. The precipitated solids were collected by filtration and washed with water (5 mL) just once and dried under reduced pressure to afford N⁸-methyl-5-(6-morpholino-[1,2,4]triazolo[1,5-a]pyridin-2-yl)pyrido[3,4-c]pyridazine-3,8-diamine as a brown solid (80.0 mg, 94.4%). LCMS (ESI) m/z 378.2, [M+H]⁺.

### Step 2: 1-fluoro-N-(8-(methylamino)-5-(6-morpholino-[1,2,4]triazolo[1,5-a]pyridin-2-yl)pyrido[3,4-c]pyridazin-3-yl)cyclopropane-1-carboxamide

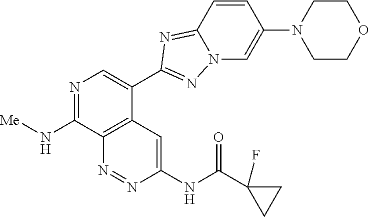 [SMILES: CNc1ncc(-c2nc3ccc(N4CCOCC4)cn3n2)c2cc(NC(=O)C3(F)CC3)nnc12]

To a stirred solution of N-(8-methyl-5-(6-morpholino-[1,2,4]triazolo[1,5-a]pyridin-2-yl)pyrido[3,4-c]pyridazine-3,8-diamine (80.0 mg; 0.210 mmol; 1.00 eq.) and 1-fluorocyclopropane-1-carboxylic acid (22.0 mg; 0.210 mmol; 1.00 eq.) in pyridine (5 mL) was added POCl₃ (100.0 mg; 0.650 mmol; 3.08 eq.) at 0° C. under nitrogen atmosphere. The resulting mixture was stirred at 0° C. for 0.5 hour. The desired product was detected via LCMS. The reaction was quenched by the addition of water (15 mL) at 0° C. The pyridine was removed under reduced pressure to precipitate solids. The precipitated solids were collected by filtration and washed with water (2 mL) just once to afford the crude product. The crude product was purified by flash chromatography on silica gel column using 2-6% of methanol in dichloromethane as eluent to afford 1-fluoro-N-(8-(methylamino)-5-(6-morpholino-[1,2,4]triazolo[1,5-a]pyridin-2-yl)pyrido[3,4-c]pyridazin-3-yl)cyclopropane-1-carboxamide (55.1 mg, 56.0%) as a yellow solid. LCMS (ESI) m/z 464.2, [M+H]⁺. ¹H NMR (400 MHz, DMSO-d₆) δ 11.21 (s, 1H), 9.75 (s, 1H), 9.10-9.00 (m, 1H), 8.91. (s, 1H), 8.35 (d, J=1.6 Hz, 1H), 7.80-7.66 (m, 2H), 3.85-3.74 (m, 4H), 3.22-3.15 (m, 4H), 3.14 (d, J=4.4 Hz, 3H), 1.60-1.40 (m, 4H).

### Example 887: Synthesis of N-(5-(8-fluoro-[1,2,4]triazolo[1,5-a]pyridin-2-yl)-8-(methylamino)pyrido[3,4-c]pyridazin-3-yl)cyclopropanecarboxamide

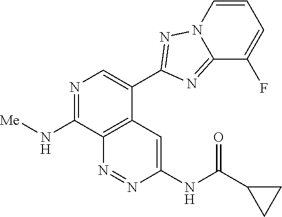 [SMILES: CNc1ncc(-c2nc3c(F)cccn3n2)c2cc(NC(=O)C3CC3)nnc12]

### Step 1: 2-chloro-8-fluoro-[1,2,4]triazolo[1,5-a]pyridine

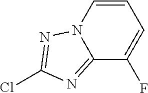 [SMILES: Fc1cccn2nc(Cl)nc12]

To a stirred solution of 8-fluoro-[1,2,4]triazolo[1,5-a]pyridin-2-amine (200.0 mg; 1.31 mmol; 1.00 eq.) and CuCl₂ (46.0 mg; 0.34 mmol; 0.26 eq.) in hydrochloric acid (12 M; 2.93 mL) was added a solution of NaNO₂ (109.0 mg; 1.58 mmol; 1.20 eq.) in water (0.6 mL) dropwise at 5° C. The mixture was stirred at 5° C. for 30 minutes and stirred at room temperature for 14 hours. The yellow mixture was diluted with water (80 mL). The resulting precipitate was filtered, rinsed with water and dried under reduced pressure to provide 2-chloro-8-fluoro-[1,2,4]triazolo[1,5-a]pyridine (121 mg, 53.6%) as a white solid. LCMS (ESI) m/z 171.0, [M+H]⁺.

### Step 2: N-(5-(8-fluoro-[1,2,4]triazolo[1,5-a]pyridin-2-yl)-8-(methylamino)pyrido[3,4-c]pyridazin-3-yl)cydopropanecarboxamide

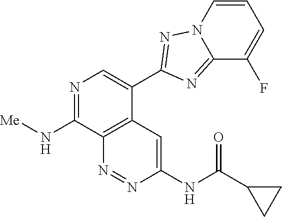 [SMILES: CNc1ncc(-c2nc3c(F)cccn3n2)c2cc(NC(=O)C3CC3)nnc12]

To a solution of N-(8-(methylamino)-5-(4,4,5,5-tetramethyl-1,3,2-dioxaborolan-2-yl)pyrido[3,4-c]pyridazin-3-yl)cyclopropanecarboxamide (Example 886, step 1) (60.0 mg; 0.16 mmol; 1.00 eq.) and 2-chloro-8-fluoro-[1,2,4]triazolo[1,5-a]pyridine (28.0 mg; 0.1.6 mmol; 1.00 eq.) in a mixture solvent of 1,4-dioxane/water (5:1, 6 mL) was added XPhos (15.0 mg; 0.03 mmol; 0.19 eq.), XPhos Pd C:3 (28.0 mg; 0.03 mmol; 0.20 eq.) and K₃PO₄ (103.1 mg; 0.48 mmol; 2.99 eq.) under nitrogen atmosphere. The resulting solution was stirred at 90° C. for 2 hours. The desired product was detected via LCMS. The resulting mixture was concentrated under reduced pressure and purified by flash chromatography on silica gel column using 5-1.0% of MeOH in CH₂Cl₂ as eluent to provide the desired product. The product was further purified by flash chromatography on pre-packed C18 column using 20-60% of MeCN in water (10 mmol/L NH₄HCO₃) to provide N-(5-(8-fluoro-[1,2,4]triazolo[1,5-a]pyridin-2-yl)-8-(methylamino)pyrido[3,4-c]pyridazin-3-yl)cyclopropanecarboxamide (2.4 mg, 3.9%) as a yellow solid. LCMS (ESI) m/z 379.1, [M+H]⁺. ¹H NMR (400 MHz, DMSO-d₆) δ 11.71 (s, 1H), 9.83 (s, 1H), 9.13-9.04 (m, 1H), 8.95 (s, 1H), 8.86 (d, J=6.4 Hz, 1H), 7.70-7.62 (m, 1H), 7.23-7.14 (m, 1H), 3.13 (d, J=4.4 Hz, 3H), 2.25-2.17 (m, 1H), 0.97-0.89 (m, 4H).

### Example 888: Synthesis of N-(5-(5-methoxybenzo[d]oxazol-2-yl)-8-(methylamino)pyrido[3,4-c]pyridazin-3-yl)cyclopropanecarboxamide

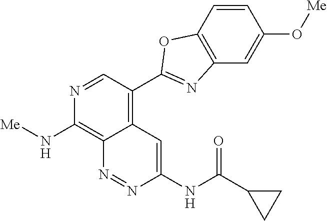 [SMILES: CNc1ncc(-c2nc3cc(OC)ccc3o2)c2cc(NC(=O)C3CC3)nnc12]

A solution of N-(8-(methylamino)-5-(4,4,5,5-tetramethyl-1,3,2-dioxaborolan-2-yl)pyrido[3,4-c]pyridazin-3-yl)cyclopropanecarboxamide (Example 886, step 1) (476.0 mg; 1.289 mmol; 1.00 eq.) and 2-chloro-5-methoxybenzo[d]oxazole (236.1 mg; 1.286 mmol; 1.00 eq.) in a mixture solvent of 1,4-dioxane/water (10:1, 8.8 mL). To the above solution was added XPhos Pd Cl₃ (109.1 mg; 0.129 mmol, 0.10 eq.), XPhos (61.4 mg; 0.129 mmol; 0.10 eq.) and K₃PO₄ (818.2 mg; 3.854 mmol; 2.99 eq.). The reaction was stirred at 90° C. for 1 hour under nitrogen atmosphere. The reaction was concentrated under reduced pressure and purified by flash chromatography on silica gel column using 5-10% of MeOH in CH₂Cl₂ as eluent to provide the crude product. The product was recrystallized twice with a mixture solvent of MeOH/CH₂Cl₂ (1:10, 11 mL) to provide N-(5-(5-methoxybenzo[d]oxazol-2-yl)-8-(methylamino)pyrido[3,4-c]pyridazin-3-yl)cyclopropanecarboxamide as a yellow solid (13.8 mg, 2.7%). LCMS (ESI) m/z 391.1, [M+H]⁺. ¹H NMR (400 MHz, DMSO-d₆) δ 11.76 (s, ¹H), 9.90 (s, ¹H), 9.40-9.30 (m, 1H), 8.92 (s, 1H), 7.63 (d, J=8.8 Hz, 1H), 7.28 (d, J=2.4 Hz, 1H), 6.97 (dd, J=8.8, 2.4 Hz, 1H), 3.86 (s, 3H), 3.16 (d, J=4.4 Hz, 3H), 2.27-2.20 (m, 1H), 1.00-0.90 (m, 4H).

### Example 889: Synthesis of N-(4-chloro-5-((5-methoxypyridin-2-yl)ethynyl)-8-(methylamino)-2,7-naphthyridin-3-yl)cyclopropanecarboxamide

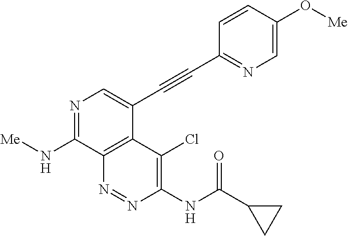 [SMILES: CNc1ncc(C#Cc2ccc(OC)cn2)c2c(Cl)c(NC(=O)C3CC3)nnc12]

### Step 1: 4-bromo-5-chloro-N¹-methyl-2,7-naphthyridine-1,6-diamine

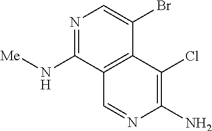 [SMILES: CNc1ncc(Br)c2c(Cl)c(N)ncc12]

A solution of 4-bromo-N¹-methyl-2,7-naphthyridine-1,6-diamine (Example 824, step 4) (200.0 mg; 0.78 mmol; 1.00 eq.) and NCS (106.0 mg; 0.78 mmol; 1.00 eq.) in DMF (1 mL) was stirred at 60° C. for 1 hour under nitrogen atmosphere. The desired product was detected via LCMS. The mixture was allowed to cool down to room temperature and diluted with brine (10 mL). The resulting mixture was extracted with EtOAc (2×10 mL). The organic layer was washed with brine (2×5 mL) and dried over anhydrous Na₂SO₄. After filtration, the filtrate was concentrated under reduced pressure. The residue was purified by flash chromatography on silica gel column using 2-6% of methanol in dichloromethane as eluent to afford 4-bromo-5-chloro-N¹-methyl-2,7-naphthyridine-1,6-diamine as a brown solid (155.0 mg, 70.4%). LCMS (ESI) m/z 287.0, [M+H]⁺.

### Step 2: N-(5-bromo-4-chloro-8-(methylamino)-2,7-naphthyridin-3-yl)cyclopropanecarboxamide

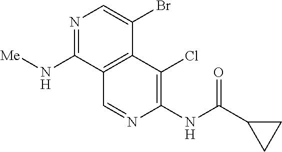 [SMILES: CNc1ncc(Br)c2c(Cl)c(NC(=O)C3CC3)ncc12]

To a solution of 4-bromo-5-chloro-N¹-methyl-2,7-naphthyridine-1,6-diamine (150.0 mg; 0.52 mmol; 1.00 eq.) and DIPEA (270.0 mg; 2.08 mmol; 4.00 eq.) in NMP (1 mL) was added cyclopropanecarbonyl chloride (110.0 mg; 1.05 mmol; 2.02 eq.) dropwise at 0° C. The mixture was stirred at room temperature for 2 hours under nitrogen atmosphere. The desired product was detected via LCMS. The resulting mixture was quenched with brine (10 mL) and extracted with EtOAc (3×10 mL). The organic layer was washed with brine (2×5 mL) and dried over anhydrous Na₂SO₄. After filtration, the filtrate was concentrated under reduced pressure. The residue was purified by flash chromatography on silica gel column using 2-6% of methanol in dichloromethane as eluent to afford N-(5-bromo-4-chloro-8-(methylamino)-2,7-naphthyridin-3-yl)cyclopropanecarboxamide as a light yellow solid (65.0 mg, 22.7%). LCMS (ESI) m/z 355.0, [M+H]⁺.

### Step 3: N-(4-chloro-5-((5-methoxypyridin-2-yl)ethynyl)-8-(methylamino)-2,7-naphthyridin-3-yl)cyclopropanecarboxamide

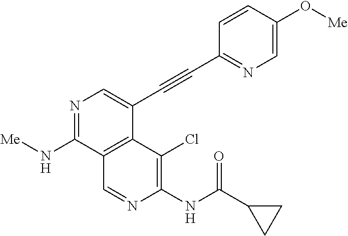 [SMILES: CNc1ncc(C#Cc2ccc(OC)cn2)c2c(Cl)c(NC(=O)C3CC3)ncc12]

To a solution of N-(5-bromo-4-chloro-8-(methylamino)-2,7-naphthyridin-3-yl)cyclopropanecarboxamide (55.3 mg; 0.20 mmol; 1.00 eq.) and 2-ethynyl-5-methoxypyridine (27.0 mg; 0.20 mmol; 1.01 eq.) in DMF (2 mL) were added Et₃N (60.0 mg; 0.59 mmol; 2.97 eq.), XPhos Pd G₃ (20.0 mg; 0.02 mmol; 0.12 eq.), XPhos (20.0 mg; 0.04 mmol; 0.21 eq.) and CuI (5.0 mg; 0.02 mmol; 0.13 eq.) at room temperature. The resulting mixture was stirred at 90° C. for 1 hour under nitrogen atmosphere. The desired product was detected via LCMS. The mixture was allowed to cool down to room temperature and concentrated under reduced pressure. The residue was purified by flash chromatography on silica gel column using 2-6% of methanol in dichloromethane as eluent to afford N-(4-chloro-5-((5-methoxypyridin-2-yl)ethynyl)-8-(methylamino)-2,7-naphthyridin-3-yl)cyclopropanecarboxamid as a yellow solid (4.4 mg, 5.4%). LCMS (ESI) m/z 408.1, [M+H]⁺. ¹H NMR (400 MHz, DMSO-d₆) a 10.59 (s, 1H), 9.36 (s, 1H), 8.65-8.55 (m, 1H), 8.44 (s, 1H), 8.30 (d, J=3.2 Hz, 1H), 7.53 (d, J=8.8 Hz, 1H), 7.43 (dd, J=8.8, 3.2 Hz, 1H), 3.87 (s, 3H), 3.05 (d, J=4.4 Hz, 3H), 1.95-1.91 (m, 1H), 0.89-0.82 (m, 4H).

### Example 890: Synthesis of (S)—N-(8-(methylamino)-5-(5-(2-methylmorpholino)benzo[d]oxazol-2-yl)pyrido[3,4-c]pyridazin-3-yl)cyclopropanecarboxamide

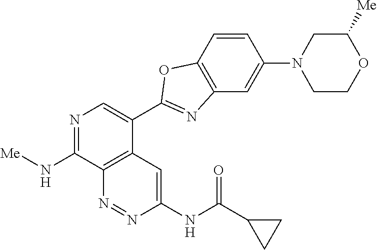 [SMILES: CNc1ncc(-c2nc3cc(N4CCO[C@@H](C)C4)ccc3o2)c2cc(NC(=O)C3CC3)nnc12]

(S)—N-(8-(methylamino)-5-(5-(2-methylmorpholino)benzo[d]oxazol-2-yl)pyrido[3,4-c]pyridazin-3-yl)cyclopropanecarboxamide was synthesized using a similar procedure that was previously described in Example 345, by using N-(5-bromo-8-(methylamino)pyrido[3,4-c]pyridazin-3-yl)cyclopropanecarboxamide and (S)-5-(2-methylmorpholino)benzo[d]oxazole as the starting material. LCMS (ESI) m/z 460.2, [M+H]⁺. ¹H NMR (400 MHz, DMSO-d₆) δ 11.78 (s, 1H), 9.91 (s, 1H), 9.40-9.33 (m, 1H), 8.92 (s, 1H), 7.59 (d, J=8.8 Hz, 1H), 7.22 (d, J=2.4 Hz, 1H), 7.07 (dd, J=8.8, 2.4 Hz, 1H), 3.98-3.90 (m, 1H), 3.76-3.59 (m, 3H), 3.57-3.50 (m, 1H), 3.15 (d, J=4.8 Hz, 3H), 2.75-2.66 (m, 1H), 2.43-2.35 (m, 1H), 2.28-2.17 (m, 1H), 1.19 (d, J=6.0 Hz, 3H), 1.02-0.88 (m, 4H).

### Example 891: Synthesis of (1S,2R)-2-methyl-N-(8-(methylamino)-5-(5-((S)-2-methylmorpholino)benzo[d]oxazol-2-yl)pyrido[3,4-c]pyridazin-3-yl)cyclopropane-1-carboxamide

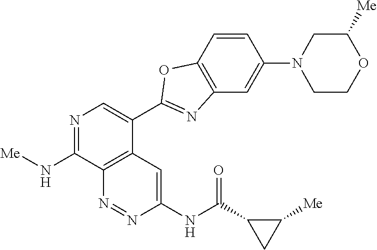 [SMILES: CNc1ncc(-c2nc3cc(N4CCO[C@@H](C)C4)ccc3o2)c2cc(NC(=O)[C@H]3C[C@H]3C)nnc12]

### Step 1: (S)—N⁸-methyl-5-(5-(2-methylmorpholino)benzo[d]oxazol-2-yl)pyrido[3,4-c]pyridazine-3,8-diamine

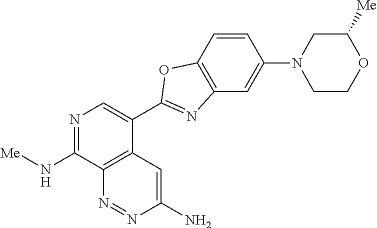 [SMILES: CNc1ncc(-c2nc3cc(N4CCO[C@@H](C)C4)ccc3o2)c2cc(N)nnc12]

To a stirred solution of (S)N-(8-(methylamino)-5-(5-(2-methylmorpholino)benzo[d]oxazol-2-yl)pyrido[3,4-c]pyridazin-3-yl)cyclopropanecarboxamide (Example 890) (100.2 mg; 0.218 mmol; 1.01 eq.) in a mixture solvent of DMSO/MeOH (3:2, 5 mL) was added a solution of NaOH: (86.5 mg; 2.163 mmol; 10.10 eq.) in water (0.5 mL). The resulting mixture was stirred at 60° C. for 2 hours under nitrogen atmosphere. The desired product was detected via LCMS. The mixture was allowed to cool down to room temperature. The MeOH was removed under reduced pressure. The resulting mixture was diluted with water (1 mL). The precipitated solids were collected by filtration and washed with water (0.5 mL) to afford (S)—N⁸-methyl-5-(5-(2-methylmorpholino)benzo[d]oxazol-2-yl)pyrido[3,4-c]pyridazine-3,8-diamine as a yellow solid (70.0 mg, 82.1%). LCMS (ESI) m/z 392.2, [M+H]⁺.

### Step 2: (1S,2R)-2-methyl-N-(8-(methylamino)-5-(5-((S)-2-methylmorpholino)benzo[d]oxazol-2-yl)pyrido[3,4-c]pyridazin-3-yl)cyclopropane-1-carboxamide

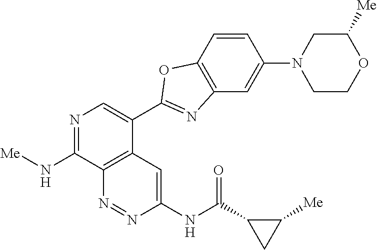 [SMILES: CNc1ncc(-c2nc3cc(N4CCO[C@@H](C)C4)ccc3o2)c2cc(NC(=O)[C@H]3C[C@H]3C)nnc12]

To a stirred solution of (S) N⁸-methyl-5-(5-(2-methylmorpholino)benzo[d]oxazol-2-yl)pyrido[3,4-c]pyridazine-3,8-diamine (70.1 mg; 0.179 mmol; 1.01 eq.) and (1S,2R)-2-methylcyclopropane-1-carboxylic acid (16.2 mg; 0.160 mmol; 0.98 eq.) in pyridine (3 mL) was added POCl₃ (81.2 mg; 0.528 mmol; 3.03 eq.) dropwise at 0° C. under nitrogen atmosphere. The resulting mixture was stirred at room temperature for 0.5 hour. The desired product was detected via LCMS. The reaction was quenched with saturated NaHCO₃ solution (1 mL) at 0° C. and concentrated under reduced pressure. The residue was purified by flash chromatography on silica gel column using 5-8% of MeOH in CH₂Cl₂ as eluent to provide a crude product. The crude product was re-crystallized with a mixture solvent of DCM/MeOH (1/1, 3 mL) to afford (1S,2R)-2-methyl N-(8-(methylamino)-5-(5-((S)-2-methylmorpholino)benzo[d]oxazol-2-yl)pyrido[3,4-c]pyridazin: 3-yl)cyclopropane-1-carboxamide as a yellow solid (37.4 mg, 44.1%). LCMS (ESI) m/z 474.2, [M+H]⁺. ¹H NMR (400 MHz, DMSO-d₆) δ 11.66 (s, 1H), 9.88 (s, 1H), 9.49-9.20 (m, 1H), 8.92 (s, 1H), 7.59 (d, J=8.8 Hz, 1H), 7.24 (d, J=2.4 Hz, 1H), 7.08 (dd, J=8.8, 2.4 Hz, 1H),3.98-3.89 (m, 1H), 3.72-3.61 (m, 3H), 3.58-3.51 (m, 1H), 3.15 (d, J=4.8 Hz, 3H), 2.77-2.60 (m, 1H), 2.43-2.36 (m, 1H), 2.29-2.18 (m, 1H), 1.44-1.38 (m, 1H), 1.21-1.17 (m, 6H), 1.12-1.04 (m, 1H), 1.00-0.90 (m, 1H).

### Example 892

### Synthesis of N⁸-methyl-5-(6-morpholino-[1,2,4]triazolo[1,5-a]pyridin-2-yl)-N³-(pyridin-2-yl)pyrido[3,4-c]pyridazine-3,8-diamine

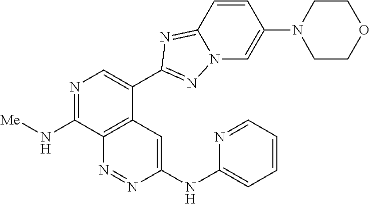 [SMILES: CNc1ncc(-c2nc3ccc(N4CCOCC4)cn3n2)c2cc(Nc3ccccn3)nnc12]

### Step 1: N⁸-methyl-5-(6-morpholino-[1,2,4]triazolo[1,5-a]pyridin-2-yl)pyrido[3,4-c]pyridazine-3,8-diamine

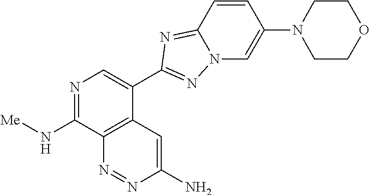 [SMILES: CNc1ncc(-c2nc3ccc(N4CCOCC4)cn3n2)c2cc(N)nnc12]

To a stirred solution of N-(8-(methylamino)-5-(6-morpholino-[1,2,4]triazolo[1,5-a]pyridin-2-yl)pyrido[3,4-c]pyridazin-3-yl)cyclopropanecarboxamide (Example 885) (64.0 mg; 0.144 mmol; 1.00 eq.) in a mixture solvent of DMSO/MeOH (2/1, 1.5 mL) was added a solution of NaOH: (58.0 mg; 1.440 mmol; 10.00 eq.) in water (0.2 mL). The resulting mixture was stirred at 60° C. overnight under nitrogen atmosphere. The desired product was detected via LCMS. The mixture was allowed to cool down to room temperature. The resulting mixture was concentrated under reduced pressure. The residue was purified by flash chromatography on pre-packed C18 column using 20-60% of MeOH in water (10 mmol/L NH₄HCO₃) to provide N⁸-methyl-5-(6-morpholino-[1,2,4]triazolo[1,5-a]pyridin-2-yl)pyrido[3,4-c]pyridazine-3,8-diamine as a yellow solid (40.0 mg, 73.8%). LCMS (ESI) m/z 378.2, [M+H]⁺.

### Step 2: N⁸-methyl-5-(6-morpholino-[1,2,4]triazolo[1,5-a]pyridin-2-yl)-N³-(pyridin-2-yl)pyrido[3,4-c]pyridazine-3,8-diamine

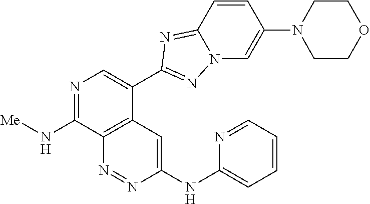 [SMILES: CNc1ncc(-c2nc3ccc(N4CCOCC4)cn3n2)c2cc(Nc3ccccn3)nnc12]

A mixture of N⁸-methyl-5-(6-morpholino-[1,2,4]triazolo[1, 5-a]pyridin-2-yl)pyrido[3,4-c]pyridazine-3,8-diamine (40.0 mg; 0.106 mmol; 1.00 eq.), 2-chloropyridine (12.0 mg; 0.106 mmol; 1.00 eq.), Pd₂(dba)₃ (11.6 mg; 0.013 mmol; 0.12 eq.), XantPhos (14.7 mg; 0.025 mmol; 0.24 eq.) in 1,4-dioxane (1 mL) was stirred at 130° C. for 2 hours under nitrogen atmosphere. The mixture was allowed to cool down to room temperature. The resulting mixture was concentrated under reduced pressure. The residue was purified by flash chromatography on silica gel column using 5-8% of MeOH in CH₂Cl₂ as eluent to provide the crude product. The crude product was re-crystallized with a mixture solvent of CH₂Cl₂/MeOH (3/1, 5 mL) to afford N⁸-methyl-5-(6-morpholino-[1,2,4]triazolo[1,5-a]pyridin-2-yl) N³-(pyridin-2-yl)pyrido[3,4-c]pyridazine-3,8-diamine as a yellow solid (13.1 mg, 27.2%). LCMS (ESI) m/z 455.2, 1H NMR (400 MHz, DMSO-d₆) δ 10.60 (s, 1H), 10.02 (s, 1H), 8.93-8.70 (m, 2H), 8.51-8.37 (m, 1H), 8.34 (d, J=2.0 Hz, 1H), 7.88-7.59 (m, 3H), 7.46 (d, J=8.4 Hz, 1H), 7.07-6.86 (m, 1H), 3.92-3.61 (m, 4H), 3.23-3.17 (m, 4H), 3.11 (d, J=4.8 Hz, 3H).

### Example 893: Synthesis of N-(5-((4-((1-acetylazetidin-3-yl)oxy)phenyl)ethynyl)-8-(methylamino)pyrido[3,4-c]pyridazin-3-yl)cyclopropanecarboxamide

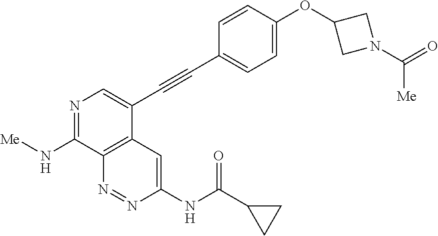 [SMILES: CNc1ncc(C#Cc2ccc(OC3CN(C(C)=O)C3)cc2)c2cc(NC(=O)C3CC3)nnc12]

### Step 1: 1-(3-(4-bromophenoxy)azetidin-1-yl)ethan-1-one

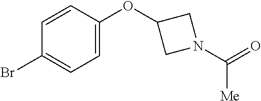 [SMILES: CC(=O)N1CC(Oc2ccc(Br)cc2)C1]

To a stirred solution of 3-(4-bromophenoxy)azetidine hydrochloride (Example 715, step 2) (1.05 g; 3.993 mmol; 1.00 eq.) and DIEA (1.54 g; 11.979 mmol; 3.00 eq.) in CH₂Cl₂ (10 mL) was added acetic anhydride (814.6 mg; 7.986 mmol; 2.00 eq.). The reaction mixture was stirred at room temperature for 2 hours. LC-MS indicated that the starting material was consumed. The resulting mixture was concentrated under vacuum. The residue was purified by flash chromatography on silica gel column using 30-50% of EtOAc in petroleum ether as eluent to provide 1-(3-(4-bromophenoxy)azetidin-1-yl)ethan-1-one as a white solid (1.02 g, 94.9%). LCMS (ESI) m/z 270.1, [M+H]⁺.

### Step 2: 1-(3-(4-((trimethylsilyl)ethynyl)phenoxy)azetidin-1-yl)ethan-1-one

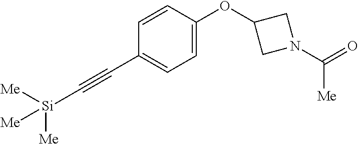 [SMILES: CC(=O)N1CC(Oc2ccc(C#C[Si](C)(C)C)cc2)C1]

A stirred solution of 1-(3-(4-bromophenoxy)azetidin-1-yl)ethan-1-one (650.0 mg; 2.406 mmol; 1.00 eq.), ethynyltrimethylsilane (472.7 mg; 4.812 mmol; 2.00 eq.), Pd(dppf)Cl₂·CH₂Cl₂ (980.1 mg; 1.203 mmol; 0.50 eq.), DIEA (933.0 mg; 7.218 mmol; 3.00 eq.) and CuI (229.1 mg; 1.203 mmol; 0.50 eq.) in DMF (7 mL) was stirred at 50° C. for 6 hours under nitrogen atmosphere. The desired product was detected via LCMS. The mixture was allowed to cool down to room temperature. The resulting mixture was concentrated under reduced pressure. The residue was purified by flash chromatography on silica gel column using 5-8% of MeOH in CH₂Cl₂ as eluent to provide the crude product. The crude product was purified by flash chromatography on pre-packed C18 column using 20-60% of MeOH in water (10 mmol/L NH₄HCO₃) to provide 1-(3-(4-((trimethylsilyl)ethynyl)phenoxy)azetidin-1-yl)ethan-1-one as an off-white solid (450.0 mg, 65.1.%).LCMS (ESI) m/z 288.1, [M+H]⁺.

### Step 3: 1-(3-(4-ethynylphenoxy)azetidin-1-yl)ethan-1-one

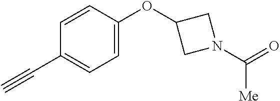 [SMILES: C#Cc1ccc(OC2CN(C(C)=O)C2)cc1]

To a solution of 1-(3-(4-((trimethylsilyl)ethynyl)phenoxy)azetidin-1-yl)ethan-1-one (180.0 mg; 0.626 mmol; 1.00 eq.) in MeOH (2 mL) was added potassium carbonate (261.5 mg; 1.878 mmol; 3.00 eq.) at room temperature. The resulting solution was stirred at room temperature for 1 hour. The desired product was detected via LCMS. The resulting mixture was diluted with water (2 mL) and extracted with EtOAc (3×10 mL). The combined organic layers were washed with water (3×10 mL), dried over anhydrous Na₂SO₄, filtered, and concentrated under reduced pressure. The residue was purified by flash chromatography, on silica gel column, using 3-5% of MeOH in CH₂Cl₂ as eluent to provide 1-(3-(4-ethynylphenoxy)azetidin-1-yl)ethan-1-one as an off-white solid (100.0 mg, 74.2%). LCMS (ESI) m/z 216.1, [M+H]⁺.

### Step 4: N-(5-((4-((1-acetylazetidin-3-yl)oxy)phenyl)ethynyl)-8-(methylamino)pyrido[3,4-c]pyridazin-3-yl)cyclopropanecarboxamide

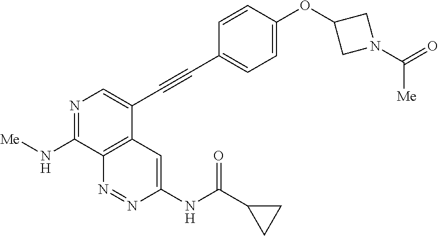 [SMILES: CNc1ncc(C#Cc2ccc(OC3CN(C(C)=O)C3)cc2)c2cc(NC(=O)C3CC3)nnc12]

A solution of N-(5-bromo-8-(methylamino)pyrido[3,4-c]pyridazin-3-yl)cyclopropanecarboxamide (70.0 mg; 0.217 mmol; 1.00 eq.), 1-(3-(4-ethynylphenoxy)azetidin-1-yl)ethan-1-one (70.2 mg; 0.326 mmol; 1.50 eq.), XPhos Pd G₃ (18.4 mg; 0.022 mmol; 0.10 eq.), XPhos (20.7 mg; 0.043 mmol; 0.20 eq.), Et₃N (109.9 mg; 1.085 mmol; 5.00 eq.) and CuI (8.2 mg; 0.043 mmol; 0.20 eq.) in DMF (1 mL) was stirred at 110° C. for 1 hour under nitrogen atmosphere. The resulting mixture was concentrated under reduced pressure. The residue was purified by flash chromatography on silica gel column using 2-10% of MeOH in CH₂Cl₂ to afford the crude product. The crude product was re-crystallized with a mixture solvent of CH₂Cl₂/MeOH (3/1, 5 mL) to afford N-(5-((4-((1-acetylazetidin-3-yl)oxy)phenyl)ethynyl)-8-(methylamino)pyrido[3,4-c]pyridazin-3-yl)cyclopropanecarboxamide as a yellow solid (27.7 mg, 27.9%). LCMS (ESI) m/z 457.2, [M+H]⁺. ¹H NMR (400 MHz, DMSO-d₆) δ 1.1.82 (s, 1H), 9.11-8.90 (m, 1H), 8.77 (s, 1H), 8.28 (s, 1H), 7.51 (d, J=8.4 Hz, 2H), 6.94 (d, J=8.4 Hz, 2H), 5.22-4.86 (m, 1H), 4.68-4.41 (m, 1H), 4.37-4.16 (m, 1H), 4.16-4.02 (m, 1H), 4.00-3.60 (m, 1H), 3.08 (d, J=4.8 Hz, 3H), 2.25-2.12 (m, 1H), 1.80 (s, 3H), 0.98-0.82 (m, 4H)

### Examples 894-912

Each compound in Table 28 below was prepared using a similar experimental procedure to prepare Example 595, using N-(5-ethynyl-8-(methylamino)-2,7-naphthyridin-3-yl)cyclopropanecarboxamide as the common intermediate and appropriate aryl halide.

| | | |
|---|---|---|
| Example 894 | 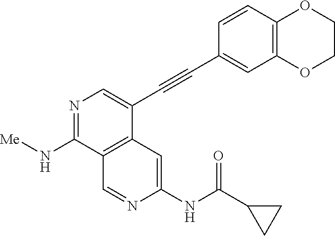 [SMILES: CNc1ncc(C#Cc2ccc3c(c2)OCCO3)c2cc(NC(=O)C3CC3)ncc12] | LCMS (ESI) m/z 401.2, [M + H]⁺. ¹H NMR (400 MHz, DMSO-d₆) δ 11.06 (s, 1H), 9.36 (s, 1H), 8.62 (s, 1H), 8.37-8.26 (m, 1H), 8.24 (s, 1H), 7.14-6.98 (m, 2H), 6.98-6.89 (m, 1H), 4.38-4.24 (m, 4H), 3.01 (d, J = 4.4 Hz, 3H), 2.15-2.03 (m, 1H), 0.96- 0.79 (m, 4H). |
| | N-(5-((2,3-dihydrobenzo[b][1,4]dioxin- | |
| | 6-yl)ethynyl)-8-(methylamino)-2,7- | |
| | naphthyridin-3- | |
| | yl)cyclopropanecarboxamide | |
| Example 895 | 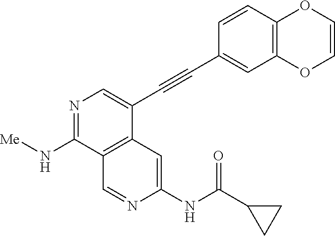 [SMILES: CNc1ncc(C#Cc2ccc3c(c2)OC=CO3)c2cc(NC(=O)C3CC3)ncc12] | LCMS (ESI) m/z 399.1, [M + H]⁺. ¹H NMR (400 MHz, DMSO-d₆) δ 11.07 (s, 1H), 9.36 (s, 1H), 8.60 (s, 1H), 8.37-8.28 (m, 1H), 8.22 (s, 1H), 7.07 (d, J = 8.4, 1.6 Hz, 1H), 6.87 (d, J = 1.6 Hz, 1H), 6.78 (d, J = 8.4 Hz, 1H), 6.25 (s, 2H), 3.01 (d, J = 4.4 Hz, 3H), 2.12-2.02 (m, 1H), 0.96- 0.80 (m, 4H). |
| | N-(5-(benzo[b][1,4]dioxin-6-ylethynyl)- | |
| | 8-(methylamino)-2,7-naphthyridin-3- | |
| | yl)cyclopropanecarboxamide | |
| Example 896 | 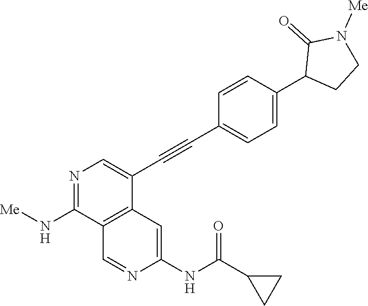 [SMILES: CNc1ncc(C#Cc2ccc(C3CCN(C)C3=O)cc2)c2cc(NC(=O)C3CC3)ncc12] | LCMS (ESI) m/z 440.3, [M + H]⁺. ¹H NMR (400 MHz, DMSO-d₆) δ 11.08 (s, 1H), 9.37 (s, 1H), 8.67 (s, 1H), 8.41-8.29 (m, 1H), 8.28 (s, 1H), 7.51 (d, J = 8.4 Hz, 2H), 7.30 (d, J = 8.4 Hz, 2H), 3.69 (t, J = 8.8 Hz, 1H), 3.53- 3.35 (m, 2H), 3.02 (d, J = 4.4 Hz, 3H), 2.82 (s, 3H), 2.49-2.40 (m, 1H), 2.18-1.97 (m, 2H), 0.95- 0.88 (m, 2H), 0.87-0.77 (m, 2H). |
| | N-(5-((4-(1-methyl-2-oxopyrrolidin-3- | |
| | yl)phenyl)ethynyl)-8-(methylamino)- | |
| | 2,7-naphthyridin-3- | |
| | yl)cyclopropanecarboxamide | |
| Example 897 | 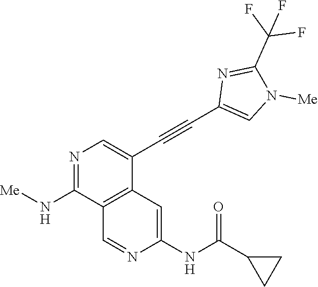 [SMILES: CNc1ncc(C#Cc2cn(C)c(C(F)(F)F)n2)c2cc(NC(=O)C3CC3)ncc12] | LCMS (ESI) m/z 415.1, [M + H]⁺. ¹H NMR (400 MHz, DMSO-d₆) δ 11.06 (s, 1H), 9.37 (s, 1H), 8.52 (s, 1H), 8.38-8.30 (m, 1H), 8.27 (s, 1H), 7.86 (s, 1H), 3.84 (s, 3H), 3.02 (d, J = 4.4 Hz, 3H), 2.13-1.80 (m, 1H), 0.91-0.79 (m, 4H). |
| | N-(5-((1-methyl-2-(trifluoromethyl)- | |
| | 1H-imidazol-4-yl)ethynyl)-8- | |
| | (methylamino)-2,7-naphthyridin-3- | |
| | yl)cyclopropanecarboxamide | |
| Example 898 | 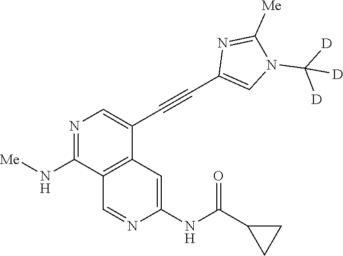 [SMILES: [2H]C([2H])([2H])n1cc(C#Cc2cnc(NC)c3cnc(NC(=O)C4CC4)cc23)nc1C] | LCMS (ESI) m/z 364.2, [M + H]⁺. ¹H NMR (400 MHz, DMSO-d₆) δ 11.04 (s, 1H), 9.35 (s, 1H), 8.52 (s, 1H), 8.28-8.22 (m, 1H), 8.20 (s, 1H), 7.39 (s, 1H), 3.01 (d, J = 4.4 Hz, 3H), 2.29 (s, 3H), 2.10-2.03 (m, 1H), 0.89-0.82 (m, 4H). |
| | N-(5-((2-methyl-1-(methyl-d₃)-1H- | |
| | imidazol-4-yl)ethynyl)-8- | |
| | (methylamino)-2,7-naphthyridin-3- | |
| | yl)cyclopropanecarboxamide | |
| Example 899 | 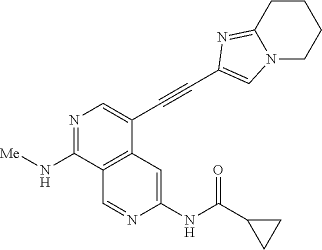 [SMILES: CNc1ncc(C#Cc2cn3c(n2)CCCC3)c2cc(NC(=O)C3CC3)ncc12] | LCMS (ESI) m/z 387.2, [M + H]⁺. ¹H NMR (400 MHz, DMSO-d₆) δ 11.04 (s, 1H), 9.36 (s, 1H), 8.53 (s, 1H), 8.28-8.23 (m, 1H), 8.20 (s, 1H), 7.36 (s, 1H), 3.96 (t, J = 6.0 Hz, 2H), 3.01 (d, J = 4.4 Hz, 3H), 2.73 (t, J = 6.0 Hz, 2H), 2.11-2.03 (m, 1H), 1.96-1.81 (m, 4H), 0.95-0.79 (m, 4H). |
| | N-(8-(methylamino)-5-((5,6,7,8- | |
| | tetrahydroimidazo[1,2-a]pyridin-2- | |
| | yl)ethynyl)-2,7-naphthyridin-3- | |
| | yl)cyclopropanecarboxamide | |
| Example 900 | 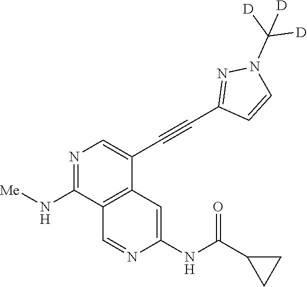 [SMILES: [2H]C([2H])([2H])n1ccc(C#Cc2cnc(NC)c3cnc(NC(=O)C4CC4)cc23)n1] | LCMS (ESI) m/z 350.2, [M + H]⁺. ¹H NMR (400 MHz, DMSO-d₆) δ 11.09 (s, 1H), 9.40 (s, 1H), 8.56 (s, 1H), 8.55-8.40 (m, 1H), 8.25 (s, 1H), 7.79 (d, J = 0.8 Hz, 1H), 6.51 (d, J = 0.8 Hz, 1H), 3.03 (d, J = 4.0 Hz, 3H), 2.16- 2.01 (m, 1H), 0.92-0.79 (m, 4H). |
| | N-(5-((1-(methyl-d₃)-1H-pyrazol-3- | |
| | yl)ethynyl)-8-(methylamino)-2,7- | |
| | naphthyridin-3- | |
| | yl)cyclopropanecarboxamide | |
| Example 901 | 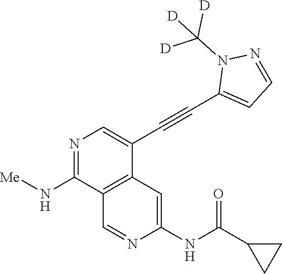 [SMILES: [2H]C([2H])([2H])n1nccc1C#Cc1cnc(NC)c2cnc(NC(=O)C3CC3)cc12] | LCMS (ESI) m/z 350.2, [M + H]⁺. ¹H NMR (400 MHz, DMSO-d₆) δ 11.11 (s, 1H), 9.38 (s, 1H), 8.61 (s, 1H), 8.52-8.38 (m, 1H), 8.33 (s, 1H), 7.52 (d, J = 2.0 Hz, 1H), 6.59 (d, J = 2.0 Hz, 1H), 3.03 (d, J = 4.4 Hz, 3H), 2.10- 2.04 (m, 1H), 0.90-0.82 (m, 4H). |
| | N-(5-((1-(methyl-d₃)-1H-pyrazol-5- | |
| | yl)ethynyl)-8-(methylamino)-2,7- | |
| | naphthyridin-3- | |
| | yl)cyclopropanecarboxamide | |
| Example 902 | 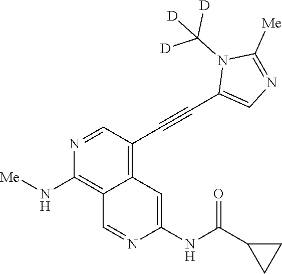 [SMILES: [2H]C([2H])([2H])n1c(C#Cc2cnc(NC)c3cnc(NC(=O)C4CC4)cc23)cnc1C] | LCMS (ESI) m/z 364.2, [M + H]⁺. ¹H NMR (400 MHz, DMSO-d₆) δ 11.07 (s, 1H), 9.37 (s, 1H), 8.58 (s, 1H), 8.37-8.30 (m, 1H), 8.27 (s, 1H), 7.15 (s, 1H), 3.02 (d, J = 4.4 Hz, 3H), 2.35 (s, 3H), 2.11-2.04 (m, 1H), 0.91-0.78 (m, 4H). |
| | N-(5-((2-methyl-1-(methyl-d₃)-1H- | |
| | imidazol-5-yl)ethynyl)-8- | |
| | (methylamino)-2,7-naphthyridin-3- | |
| | yl)cyclopropanecarboxamide | |
| Example 903 | 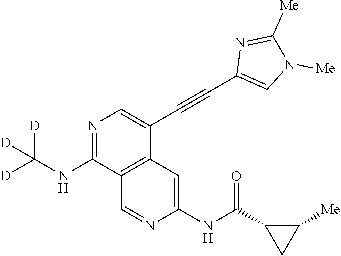 [SMILES: [2H]C([2H])([2H])Nc1ncc(C#Cc2cn(C)c(C)n2)c2cc(NC(=O)[C@H]3C[C@H]3C)ncc12] | LCMS (ESI) m/z 378.2, [M + H]⁺. ¹H NMR (400 MHz, DMSO-d₆) δ 10.92 (s, 1H), 9.34 (s, 1H), 8.51 (s, 1H), 8.21 (s, 1H), 8.19 (s, 1H), 7.40 (s, 1H), 3.58 (s, 3H), 2.29 (s, 3H), 2.12-2.02 (m, 1H), 1.38-1.26 (m, 1H), 1.13 (d, J = 6.0 Hz, 3H), 1.03-0.93 (m, 1H), 0.89-0.79 (m, 1H). |
| | (1S,2R)-N-(5-((1,2-dimethyl-1H- | |
| | imidazol-4-yl)ethynyl)-8-((methyl- | |
| | d₃)amino)-2,7-naphthyridin-3-yl)-2- | |
| | methylcyclopropane-1-carboxamide | |
| Example 904 | 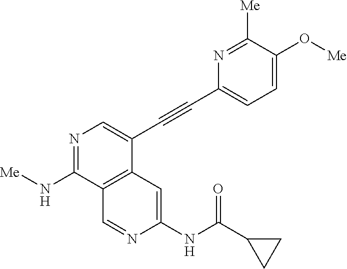 [SMILES: CNc1ncc(C#Cc2ccc(OC)c(C)n2)c2cc(NC(=O)C3CC3)ncc12] | LCMS (ESI) m/z 388.2, [M + H]⁺. ¹H NMR (400 MHz, DMSO-d₆) δ 11.07 (s, 1H), 9.37 (s, 1H), 8.62 (s, 1H), 8.37-8.32 (m, 1H), 8.30 (s, 1H), 7.48 (d, J = 8.4 Hz, 1H), 7.41 (d, J = 8.8 Hz, 1H), 3.87 (s, 3H), 3.02 (d, J = 4.4 Hz, 3H), 2.38 (s, 3H), 2.14-2.02 (m, 1H), 0.94-0.75 (m, 4H). |
| | N-(5-((5-methoxy-6-methylpyridin-2- | |
| | yl)ethynyl)-8-(methylamino)-2,7- | |
| | naphthyridin-3- | |
| | yl)cyclopropanecarboxamide | |
| Example 905 | 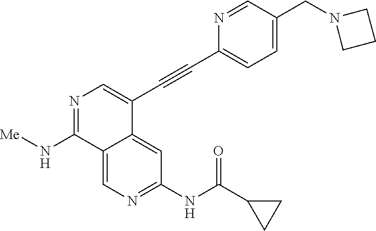 [SMILES: CNc1ncc(C#Cc2ccc(CN3CCC3)cn2)c2cc(NC(=O)C3CC3)ncc12] | LCMS (ESI) m/z 413.2, [M + H]⁺. ¹H NMR (400 MHz, DMSO-d₆) δ 11.08 (s, 1H), 9.39 (s, 1H), 8.62 (s, 1H), 8.50 (d, J = 2.0 Hz, 1H), 8.45-8.38 (m, 1H), 8.34 (s, 1H), 7.75 (dd, J = 8.0, 2.0 Hz, 1H), 7.58 (d, J = 8.0 Hz, 1H), 3.63 (s, 2H), 3.25-3.14 (m, 4H), 3.04 (d, J = 4.4 Hz, 3H), 2.14- 2.05 (m, 1H), 2.09-1.97 (m, 2H), 0.94-0.80 (m, 4H). |
| | N-(5-((5-(azetidin-1-ylmethyl)pyridin-2- | |
| | yl)ethynyl)-8-(methylamino)-2,7- | |
| | naphthyridin-3- | |
| | yl)cyclopropanecarboxamide | |
| Example 906 | 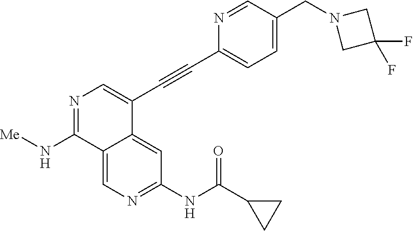 [SMILES: CNc1ncc(C#Cc2ccc(CN3CC(F)(F)C3)cn2)c2cc(NC(=O)C3CC3)ncc12] | LCMS (ESI) m/z 449.1, [M + H]⁺. ¹H NMR (400 MHz, DMSO-d₆) δ 11.10 (s, 1H), 9.39 (s, 1H), 8.62 (s, 1H), 8.54 (s, 1H), 8.45- 8.40 (m, 1H), 8.35 (s, 1H), 7.79 (d, J = 8.0 Hz, 1H), 7.60 (d, J = 8.0 Hz, 1H), 3.80 (s, 2H), 3.66 (t, J = 12.4 Hz, 4H), 3.04 (d, J = 4.4, 3H), 2.13-2.05 (m, 1H), 0.94-0.80 (m, 4H). |
| | N-(5-((5-((3,3-difluoroazetidin-1- | |
| | yl)methyl)pyridin-2-yl)ethynyl)-8- | |
| | (methylamino)-2,7-naphthyridin-3- | |
| | yl)cyclopropanecarboxamide | |
| Example 907 | 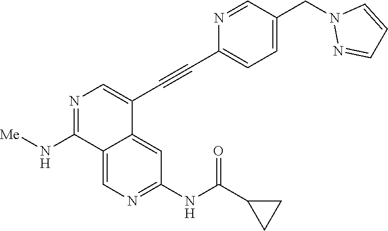 [SMILES: CNc1ncc(C#Cc2ccc(Cn3cccn3)cn2)c2cc(NC(=O)C3CC3)ncc12] | LCMS (ESI) m/z 424.2, [M + H]⁺. ¹H NMR (400 MHz, DMSO-d₆) δ 11.08 (s, 1H), 9.38 (s, 1H), 8.62 (s, 1H), 8.50 (d, J = 2.0 Hz, 1H), 8.46-8.39 (m, 1H), 8.34 (s, 1H), 7.90 (d, J = 2.0 Hz, 1H), 7.71-7.63 (m, 1H), 7.60 (d, J = 8.0 Hz, 1H), 7.51 (d, J = 2.0 Hz, 1H), 6.31 (t, J = 2.0 Hz, 1H), 5.44 (s, 2H), 3.03 (d, J = 4.4 Hz, 3H), 2.14-2.03 (m, 1H), 0.94- 0.80 (m, 4H). |
| | N-(5-((5-((1H-pyrazol-1- | |
| | yl)methyl)pyridin-2-yl)ethynyl)-8- | |
| | (methylamino)-2,7-naphthyridin-3- | |
| | yl)cyclopropanecarboxamide | |
| Example 908 | 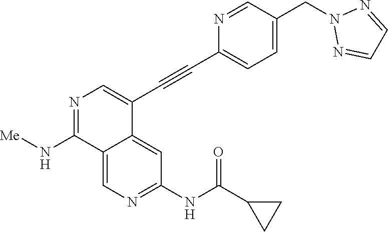 [SMILES: CNc1ncc(C#Cc2ccc(Cn3nccn3)cn2)c2cc(NC(=O)C3CC3)ncc12] | LCMS (ESI) m/z 425.1, [M + H]⁺. ¹H NMR (400 MHz, DMSO-d₆) δ 11.08 (s, 1H), 9.38 (s, 1H), 8.62 (s, 1H), 8.57 (s, 1H), 8.47- 8.39 (m, 1H), 8.35 (d, J = 2.0 Hz, 1H), 7.87 (s, 2H), 7.74 (dd, J = 8.0, 2.0 Hz, 1H), 7.62 (d, J = 8.0 Hz, 1H), 5.78 (s, 2H), 3.03 (d, J = 4.4 Hz, 3H), 2.14-2.03 (m, 1H), 0.94-0.80 (m, 4H). |
| | N-(5-((5-((2H-1,2,3-triazol-2- | |
| | yl)methyl)pyridin-2-yl)ethynyl)-8- | |
| | (methylamino)-2,7-naphthyridin-3- | |
| | yl)cyclopropanecarboxamide | |
| Example 909 | 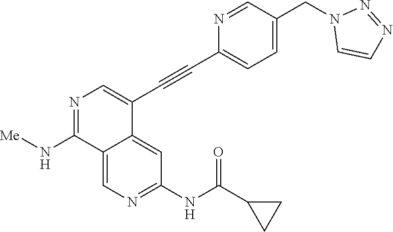 [SMILES: CNc1ncc(C#Cc2ccc(Cn3ccnn3)cn2)c2cc(NC(=O)C3CC3)ncc12] | LCMS (ESI) m/z 425.2, [M + H]⁺. ¹H NMR (400 MHz, DMSO-d₆) δ 11.09 (s, 1H), 9.38 (s, 1H), 8.62 (s, 2H), 8.48-8.40 (m, 1H), 8.35 (s, 1H), 8.27 (s, 1H), 7.82- 7.72 (m, 2H), 7.64 (d, J = 8.0 Hz, 1H), 5.74 (s, 2H), 3.04 (d, J = 4.4 Hz, 3H), 2.14-2.03 (m, 1H), 0.94-0.80 (m, 4H). |
| | N-(5-((5-((1H-1,2,3-triazol-1- | |
| | yl)methyl)pyridin-2-yl)ethynyl)-8- | |
| | (methylamino)-2,7-naphthyridin-3- | |
| | yl)cyclopropanecarboxamide | |
| Example 910 | 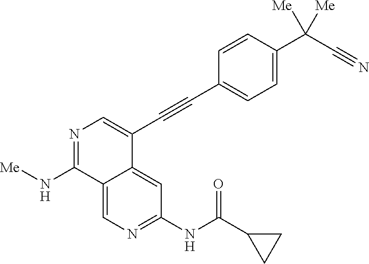 [SMILES: CNc1ncc(C#Cc2ccc(C(C)(C)C#N)cc2)c2cc(NC(=O)C3CC3)ncc12] | LCMS (ESI) m/z 410.2, [M + H]⁺. ¹H NMR (400 MHz, DMSO-d₆) δ 11.08 (s, 1H), 9.38 (s, 1H), 8.67 (s, 1H), 8.38-8.31 (m, 1H), 8.29 (s, 1H), 7.66-7.56 (m, 4H), 3.03 (d, J = 4.4 Hz, 3H), 2.15- 2.04 (m, 1H), 1.72 (s, 6H), 0.95- 0.80 (m, 4H). |
| | N-(5-((4-(2-cyanopropan-2- | |
| | yl)phenyl)ethynyl)-8-(methylamino)- | |
| | 2,7-naphthyridin-3- | |
| | yl)cyclopropanecarboxamide | |
| Example 911 | 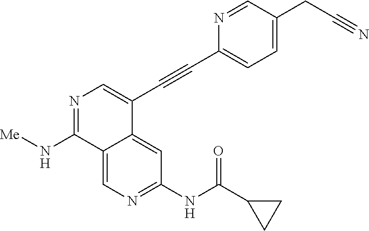 [SMILES: CNc1ncc(C#Cc2ccc(CC#N)cn2)c2cc(NC(=O)C3CC3)ncc12] | LCMS (ESI) m/z 383.2, [M + H]⁺. ¹H NMR (400 MHz, DMSO-d₆) δ 11.11 (s, 1H), 9.39 (s, 1H), 8.63 (s, 1H), 8.59 (d, J = 2.0 Hz, 1H), 8.48-8.42 (m, 1H), 8.37 (s, 1H), 7.87 (dd, J = 8.4, 2.0 Hz, 1H), 7.67 (d, J = 8.4 Hz, 1H), 4.16 (s, 2H), 3.04 (d, J = 4.4 Hz, 3H), 2.14-2.04 (m, 1H), 0.95- 0.80 (m, 4H). |
| | N-(5-((5-(cyanomethyl)pyridin-2- | |
| | yl)ethynyl)-8-(methylamino)-2,7- | |
| | naphthyridin-3- | |
| | yl)cyclopropanecarboxamide | |
| Example 912 | 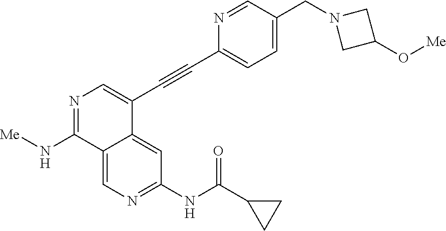 [SMILES: CNc1ncc(C#Cc2ccc(CN3CC(OC)C3)cn2)c2cc(NC(=O)C3CC3)ncc12] | LCMS (ESI) m/z 443.2, [M + H]⁺. ¹H NMR (400 MHz, DMSO-d₆) δ 11.09 (s, 1H), 9.38 (s, 1H), 8.62 (s, 1H), 8.49 (d, J = 2.0 Hz, 1H), 8.45-8.37 (m, 1H), 8.34 (s, 1H), 7.74 (dd, J = 8.0, 2.0 Hz, 1H), 7.57 (d, J = 8.0 Hz, 1H), 4.03-3.95 (m, 1H), 3.63 (s, 2H), 3.54-3.46 (m, 2H), 3.16 (s, 3H), 3.03 (d, J = 4.4 Hz, 3H), 2.93- 2.85 (m, 2H), 2.14-2.03 (m, 1H), 0.94-0.80 (m, 4H). |
| | N-(5-((5-((3-methoxyazetidin-1- | |
| | yl)methyl)pyridin-2-yl)ethynyl)-8- | |
| | (methylamino)-2,7-naphthyridin-3- | |
| | yl)cyclopropanecarboxamide | |

### Example 913 and 914

The compounds listed in Table 29 (below) were prepared using a similar experimental procedure to prepare in Example 890.

TABLE 29

| | | |
|---|---|---|
| Example 913 | 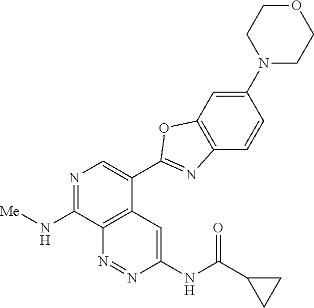 [SMILES: CNc1ncc(-c2nc3ccc(N4CCOCC4)cc3o2)c2cc(NC(=O)C3CC3)nnc12] | LCMS (ESI) m/z 446.2, [M + H]⁺. ¹H NMR (400 MHz, DMSO-d₆) δ 11.75 (s, 1H), 9.91 (s, 1H), 9.33- 9.24 (m, 1H), 8.86 (s, 1H), 7.59 (d, J = 8.8 Hz, 1H), 7.28 (d, J = 2.4 Hz, 1H) ,7.05 (dd, J = 8.8, 2.4 Hz, 1H), 3.81-3.72 (m, 4H), 3.23- 3.18 (m, 4H), 3.14 (d, J = 4.8 Hz, 3H), 2.26-2.15 (m, 1H), 1.01- 0.84 (m, 4H). |
| | N-(8-(methylamino)-5-(6- | |
| | morpholinobenzo[d]oxazol-2- | |
| | yl)pyrido[3,4-c]pyridazin-3- | |
| | yl)cyclopropanecarboxamide | |
| Example 914 | 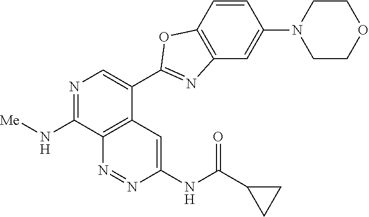 [SMILES: CNc1ncc(-c2nc3cc(N4CCOCC4)ccc3o2)c2cc(NC(=O)C3CC3)nnc12] | LCMS (ESI) m/z 446.2, [M + H]⁺. ¹H NMR (400 MHz, DMSO-d₆) δ 11.76 (s, 1H), 9.91 (s, 1H), 9.51- 9.20 (m, 1H), 8.91 (s, 1H), 7.59 (d, J = 8.8 Hz, 1H), 7.22 (d, J = 2.0 Hz, 1H), 7.07 (dd, J = 8.8, 2.0 Hz, 1H), 3.92-3.69 (m, 4H), 3.22- 3.01 (m, 7H), 2.29-2.18 (m, 1H), 1.01-0.89 (m, 4H). |
| | N-(8-(methylamino)-5-(5- | |
| | morpholinobenzo[d]oxazol-2- | |
| | yl)pyrido[3,4-c]pyridazin-3- | |
| | yl)cyclopropancarboxamide | |

### Example 915

Example 915 was prepared using a similar experimental procedure to prepare in Example 760 using Example 893 as the starting material.

| | | |
|---|---|---|
| Example 915 | 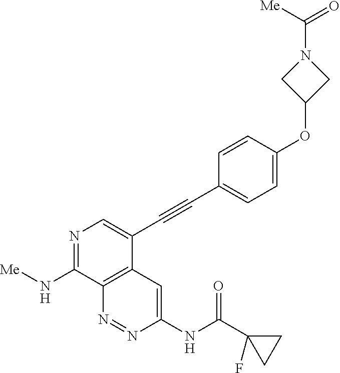 [SMILES: CNc1ncc(C#Cc2ccc(OC3CN(C(C)=O)C3)cc2)c2cc(NC(=O)C3(F)CC3)nnc12] | LCMS (ESI) m/z 475.2, [M + H]⁺. ¹H NMR (400 MHz, DMSO-d₆) δ 11.36 (s, 1H), 9.11-9.02 (m, 1H), 8.71 (s, 1H), 8.32 (s, 1H), 7.51 (d, J = 8.4 Hz, 2H), 6.93 (d, J = 8.4 Hz, 2H), 5.11-5.03 (m, 1H), 4.61- 4.53 (m, 1H), 4.36-4.27 (m, 1H), 4.15-4.08 (m, 1H), 3.84- 3.76 (m, 1H), 3.09 (d, J = 4.8 Hz, 3H), 1.80 (s, 3H), 1.59-1.40 (m, 4H). |
| | N-(5-((4-((1-acetylazetidin-3- | |
| | yl)oxy)phenyl)ethynyl)-8- | |
| | (methylamino)pyrido[3,4-c]pyridazin- | |
| | 3-yl)-1-fluorocyclopropane-1- | |
| | carboxamide | |

### Example 916: Synthesis of N-[5-[5-(4-fluoro-1-piperidyl)-1,3-benzoxazol-2-yl]-8-(methylamino)-2,7-naphthyridin-3-yl]cyclopropanecarboxamide

### Step 1. Synthesis to 5-(4-fluoro-1-piperidyl)-1,3-benzoxazole

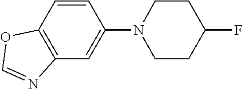 [SMILES: FC1CCN(c2ccc3ocnc3c2)CC1]

Step 1: To a solution of 5-bromo-2-methyl-1,3-benzoxazole (500 mg, 2.36 mmol), 4-fluoropiperidine (400 mg, 3.88 mmol), Brettphos (250 mg, 0.466 mmol), Cs₂CO₃ (2 g, 6.14 mmol) in dioxane (20 mL) was added BrettPhos-Pd-G₃ (200 mg, 0.221 mmol) and the reaction mixture was stirred at 100° C. for 12 hr under N₂ atmosphere. The reaction mixture was concentrated under reduced pressure. The residue was purified by flash silica gel chromatography (ISCO®; 20 g SepaFlash® Silica Flash Column, petroleum ether/EtOAc with Et(I)Ac from 0-40%, flow rate: 40 mL/min, 254 run), to provide 5-(4-Fluoro-1-piperidyl)-2-methyl-1,3-benzoxazole (400 mg, 72.4% yield) as yellow solid. LCMS (ESI) m/z 234.9 [M+H]⁺; NMR (400 MHz, DMSO-d₆) δ ppm 7.46 (d, J=8.8 Hz, 1H), 7.17 (d, J=2.3 Hz, 1H), 7.01 (dd, J=8.9, 2.4 Hz, 1H), 4.69-4.94 (m, 1H), 3.25-3.32 (m, 2H), 3.08 (ddd, J=12.0, 7.7, 3.8 Hz, 2H), 2.55 (s, 3H), 1.92-2.12 (m, 2H), 1.72-1.90 (m, 2H).

Step 2: To a solution of 5-(4-fluoro-1-piperidyl)-2-methyl-1,3-benzoxazole (400 mg, 1.71 mmol) in EtOH (10 mL) was added 4M HCl/H₂O (4.5 mL, 18.0 mmol) and the reaction mixture was stirred at 100° C. for 4 hr. The reaction was concentrated to provide 2-Amino-4-(4-fluoro-1-piperidyl)phenol (300 mg, crude) as brown oil, which was used next step directly.

Step 3: A mixture of 2-amino-4-(4-fluoro-1-piperidyl)phenol (300 mg, 1.43 mmol) in trimethoxymethane (9.68 g, 91.2 mmol) was stirred at 100° C. for 12 hrs. The reaction mixture was concentrated under reduced pressure. The residue was purified by flash silica gel chromatography (ISCO®; 20 g SepaFlash® Silica Flash Column, petroleum ether/EtOAc with EtOAc from 0-40%, flow rate: 40 mL/min, 254 nm), to provide 5-(4-Fluoro-1-piperidyl)-1,3-benzoxazole (300 mg, 95.4% yield) as brown solid. LCMS (ESI) m/z 221.1 [M+1-1]⁺; ¹H NMR (400 MHz, DMSO-d₆) δ ppm 8.60 (s, 1H), 7.58 (d, J=9.0 Hz, 1H), 7.29 (d, J=2.5 Hz, 1H), 7.14 (dd, J=8.9, 2.4 Hz, 1H), 4.74-4.94 (m, 1H), 3.28-3.32 (m, 2H), 3.06-3.17 (m, 2H), 1.91-2.10 (m, 2H), 1.74-1.89 (m, 2H).

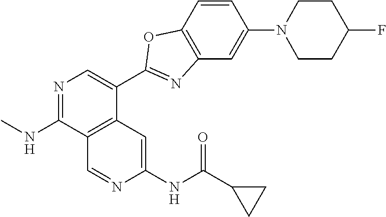 [SMILES: CNc1ncc(-c2nc3cc(N4CCC(F)CC4)ccc3o2)c2cc(NC(=O)C3CC3)ncc12]

Step 4. Synthesis of N-[5-[5-(4-fluoro-1-piperidyl)-1,3-benzoxazol-2-yl]-8-(methylamino)-2,7-naphthyridin-3-yl]cyclopropanecarboxamide To a solution of 5-(4-fluoro-1-piperidyl)-1,3-benzoxazole (100 mg, 0.454 mmol), N-[5-bromo-8-(methylamino)-2,7-naphthyridin-3-yl]cyclopropanecarboxamide (125 mg, 0.389 mmol), K₃PO₄ (300 mg, 1.41 mmol) in DMF (10 mL) was added Pd(PPh₃)₄ (100 mg, 0.087 mmol). The reaction mixture was stirred at 140° C. for 4 h under microwave conditions. The reaction mixture was concentrated under reduced pressure. The residue was purified by flash silica gel chromatography (ISCO®; 20 g SepaFlash® Silica Flash Column, petroleum ether/EtOAc with EtOAc from 01.00%, flow rate: 40 mL/min, 254 nm), to provide N-[5-[5-(4-fluoro-1-piperidyl)-1,3-benzoxazol-2-yl]-8-(methylamino)-2,7-naphthyridin-3-yl]cyclopropanecarboxamide (45 mg, 21.5% yield) as yellow solid. LCMS (ESI) m/z 461.0 [M+H]⁺; ¹H NMR (400 MHz, DMSO-d₆) δ ppm 11.03 (s, 1H), 9.63 (s, 1H), 9.42 (s, 1H), 8.86 (s, 1H), 8.67-8.58 (m, 1H), 7.55 (d, J=8.9 Hz, 1H), 7.24 (d, J=2.3 Hz, 1H), 7.07 (dd, J=9.0, 2.4 Hz, 1H), 4.75-4.95 (m, 1H), 3.52-3.41 (m, 2H), 3.14 (ddd, J=12.0, 7.7, 3.9 Hz, 2H), 3.07 (d, J=4.4 Hz, 3H), 2.13-1.96 (m, 3H), 1.93-1.79 (m, 2H), 0.82-0.95 (m, 4H).

### Examples 917-946

Each compound in Table 30 (below) was prepared using a similar experimental procedure to prepare Example 916, step 2, using N-(5-bromo-8-(methylamino)-2,7-naphthyridin-3-yl)cyclopropanecarboxamide as the common intermediate and appropriate benzoxazoles/benzothiazoles reagent.

TABLE 30

| Example | Structure and Name | MS and ¹H NMR |
|---|---|---|
| Example 917 | 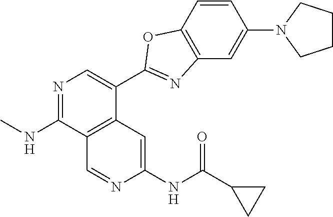 [SMILES: CNc1ncc(-c2nc3cc(N4CCCC4)ccc3o2)c2cc(NC(=O)C3CC3)ncc12] | LCMS (ESI) m/z 429.3 [M + H]⁺. ¹H NMR (400 MHz, DMSO-d₆) δ ppm 11.04 (s, 1H), 9.67 (s, 1H), 9.43 (s, 1H), 8.88 (s, 1H), 8.6 (br d, J = 4.5 Hz, 1H), 7.52 (d, J = 8.8 Hz, 1H), 6.77 (d, J = 2.3 Hz, 1H), 6.62 (dd, J = 8.9, 2.4 Hz, 1H), 3.30 (br t, J = 6.3 Hz, 4H), 3.08 (d, J = 4.3 Hz, 3H), 2.13-2.07 (m, 1H), 2.03-1.98 (m, 4H), 0.93- 0.84 (m, 4H). |
| | N-[8-(methylamino)-5-(5-pyrrolidin-1-yl- | |
| | 1,3-benzoxazol-2-yl)-2,7-naphthyridin-3- | |
| | yl]cyclopropanecarboxamide | |
| Example 918 | 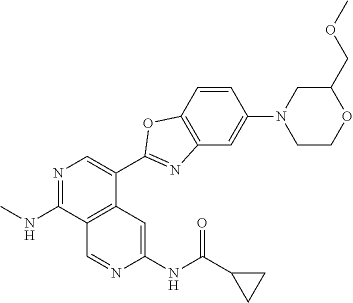 | LCMS (ESI) m/z 489.1 [M + H]⁺; ¹H NMR (400 MHz, DMSO-d₆) δ ppm 11.02 (s, 1H), 9.68 (s, 1H), 9.39 (s, 1H), 8.93 (s, 1H), 8.58 (s, 1H), 7.54-7.63 (m, 1H), 7.02 (s, 1H), 7.24 (m, 1H), 3.43-3.93 (m, 8H), 3.51 (s, 3H), 3.05 (s, 3H), 2.70-2.79 (m, 1H), 2.10 (s, 1H), 0.92 (br s, 4H). |
| | N-(5-(5-(2- | |
| | (methoxymethyl)morpholino)benzo[d]oxa- | |
| | zol-2-yl)-8-(methylamino)-2,7- | |
| | naphthyridin-3- | |
| | yl)cyclopropanecarboxamide | |
| Example 919 | 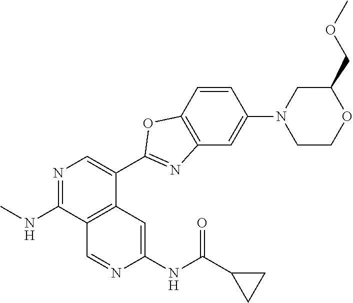 | LCMS (ESI) m/z 489.0 [M + H]⁺. ¹H NMR (400 MHz, DMSO-d₆) δ ppm 11.05 (s, 1H), 9.60 (s, 1H), 9.50 (s, 1H), 8.87 (s, 1H), 8.65 (s, 1H), 7.57 (s, 1H), 7.21 (s, 1H), 6.96 (m, 1H), 3.69-3.75 (br t, J = 10.89 Hz, 8H), 3.30 (s, 3H), 3.15 (s, 3H), 2.69-2.78 (m, 1H), 2.11 (br s, 1H), 0.82-0.94 (m, 4H). |
| | (S)-N-(5-(5-(2- | |
| | (methoxymethyl)morpholino)benzo[d]oxa- | |
| | zol-2-yl)-8-(methylamino)-2,7- | |
| | naphthyridin-3- | |
| | yl)cyclopropanecarboxamide | |
| | Isomer obtained by chiral SFC purification | |
| | of example 918; the configuration has been | |
| | arbitrarily assigned. | |
| Example 920 | 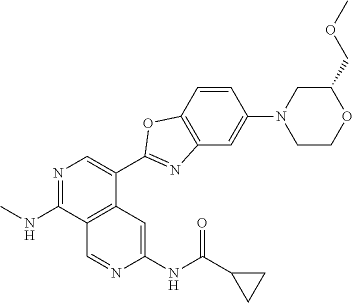 | LCMS (ESI) m/z 489.1 [M + H]⁺, ¹H NMR (400 MHz, DMSO-d₆) δ ppm 11.10 (s, 1H), 9.47 (s, 1H), 9.67 (s, 1H), 8.73 (s, 1H), 8.89 (s, 1H), 7.54-7.61 (m, 1H), 7.02 (m, 1H), 7.22 (s, 1H), 3.44 (br s, 8H), 3.28-3.32 (m, 3H), 3.07 (br s, 3H), 2.69- 2.77 (m, 1H), 2.08-2.15 (m, 1H), 0.83-0.93 (m, 4H) |
| | (R)-N-(5-(5-(2- | |
| | (methoxymethyl)morpholino)benzo[d]oxa- | |
| | zol-2-yl)-8-(methylamino)-2,7- | |
| | naphthyridin-3- | |
| | yl)cyclopropanecarboxamide | |
| | Isomer obtained by chiral SFC purification | |
| | of example 918; the configuration has been | |
| | arbitrarily assigned. | |
| Example 921 | 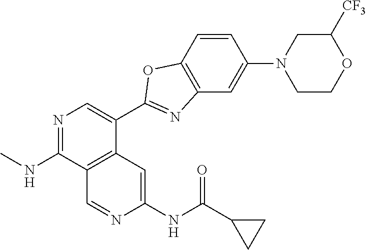 [SMILES: CNc1ncc(-c2nc3cc(N4CCOC(C(F)(F)F)C4)ccc3o2)c2cc(NC(=O)C3CC3)ncc12] | LCMS (ESI) m/z 513.2 [M + H]⁺; ¹H NMR (400 MHz, DMSO-d₆) δ ppm 11.33 (br s, 1H), 9.73 (s, 1H), 9.62-9.69 (m, 1H), 8.57-8.62 (m, 1H), 7.67 (s, 1 H), 7.37-7.41 (m, 1H), 7.20-7.25 (m, 1H), 4.40 (br d, J = 3.13 Hz, 1H), 4.11 (br d, J = 2.50 Hz, 1H), 3.77-3.85 (m, 2H), 3.61 (br d, J = 11.51 Hz, 1H), 3.17-3.22 (m, 3H), 2.78-2.92 (m, 2H), 2.13 (br s, 1H), 0.88-0.96 (m, 4H); ¹⁹F NMR (376 MHz, CD₃OD) δ ppm −78.88 (br s, 1F). |
| | N-(8-(methylamino)-5-(5-(2- | |
| | (trifluoromethyl)morpholino) | |
| | benzo[d]oxazol-2-yl)-2,7-naphthyridin-3- | |
| | yl)cyclopropanecarboxamide | |
| Example 922 | 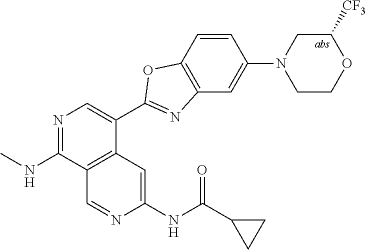 [SMILES: CNc1ncc(-c2nc3cc(N4CCO[C@@H](C(F)(F)F)C4)ccc3o2)c2cc(NC(=O)C3CC3)ncc12] | LCMS (ESI) m/z 513.2 [M + H]⁺, ¹H NMR (400 MHz, DMSO-d₆) δ ppm 11.06 (s, 1H), 9.65 (s, 1H), 9.44 (s, 1H), 8.88 (s, 1H), 8.66-8.72 (m, 1H), 7.62 (d, J = 8.80 Hz, 1H), 7.34 (d, J = 2.20 Hz, 1H), 7.14 (dd, J = 8.91, 2.31 Hz, 1H), 4.35- 4.44 (m, 1H), 4.07-4.15 (m, 1H), 3.73-3.89 (m, 2H), 3.59 (br d, J = 11.66 Hz, 1H), 3.09 (d, J = 4.40 Hz, 3H), 2.75-2.90 (m, 2H), 2.06-2.15 (m, 1H), 0.83-0.93 (m, 4 H); ¹⁹F NMR (376 MHz, DMSO-d₆) δ ppm −75.86 (br s, 1 F). |
| | (R)-N-(8-(methylamino)-5-(5-(2- | |
| | (trifluoromethyl)morpholino) | |
| | benzo[d]oxazol-2-yl)-2,7-naphthyridin-3- | |
| | yl)cyclopropanecarboxamide | |
| | Isomer obtained by chiral SFC purification | |
| | of example 921; the configuration has been | |
| | arbitrarily assigned. | |
| Example 923 | 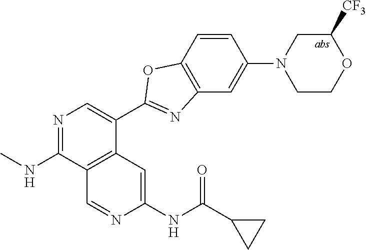 [SMILES: CNc1ncc(-c2nc3cc(N4CCO[C@H](C(F)(F)F)C4)ccc3o2)c2cc(NC(=O)C3CC3)ncc12] | LCMS (ESI) m/z 513.2 [M + H]⁺, ¹H NMR (400 MHz, DMSO-d₆) δ ppm 11.07 (s, 1H), 9.65 (s, 1H), 9.45 (s, 1H), 8.88 (s, 1H), 7.62 (d, J = 8.80 Hz, 1H), 7.35 (d, J = 2.42 Hz, 1H), 7.09-7.21 (m, 1H), 4.35-4.44 (m, 1H), 4.09-4.15 (m, 1H), 3.79-3.88 (m, 1H), 3.75 (br d, J = 10.78 Hz, 1H), 3.55-3.62 (m, 1H), 3.35 (br s, 1H), 3.07- 3.11 (m, 3H), 2.77-2.90 (m, 2H), 2.07-2.14 (m, 1H), 0.83- 0.93 (m, 4H); ¹⁹F NMR (376 MHz, DMSO-d6) δ ppm −75.86 (br s, 1 F). |
| | (S)-N-(8-(methylamino)-5-(5-(2- | |
| | (trifluoromethyl)morpholino) | |
| | benzo[d]oxazol-2-yl)-2,7-naphthyridin-3- | |
| | yl)cyclopropanecarboxamide | |
| | Isomer obtained by chiral SFC purification | |
| | of example 921; the configuration has been | |
| | arbitrarily assigned. | |
| Example 924 | 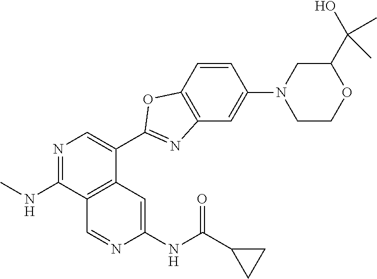 [SMILES: CNc1ncc(-c2nc3cc(N4CCOC(C(C)(C)O)C4)ccc3o2)c2cc(NC(=O)C3CC3)ncc12] | LCMS (ESI) m/z 503.4 [M + H]⁺; ¹H NMR (400 MHz, CD₃OD) δ ppm 9.75 (s, 1H), 9.25 (s, 1H), 8.87 (s, 1H), 7.55 (d, J = 8.93 Hz, 1H), 7.40 (d, J = 2.20 Hz, 1H), 7.11 (dd, J = 8.86, 2.38 Hz, 1H), 4.62 (br s, 1H), 4.11 (dd, J = 11.13, 2.32 Hz, 1H), 3.79-3.88 (m, 1H), 3.68 (br d, J = 11.37 Hz, 1H), 3.49-3.53 (m, 1H), 3.16 (s, 3H), 2.84 (br d, J = 3.42 Hz, 1H), 2.69 (t, J = 11.00 Hz, 1H), 2.00 (br t, J = 4.46 Hz, 1H), 1.30 (d, J = 9.78 Hz, 6H), 1.11 (quin, J = 3.76 Hz, 2H), 0.94- 1.00 (m, 2H). |
| | N-(5-(5-(2-(2-hydroxypropan-2- | |
| | yl)morpholino)benzo[d]oxazol-2-yl)-8- | |
| | (methylamino)-2,7-naphthyridin-3- | |
| | yl)cyclopropanecarboxamide | |
| Example 925 | 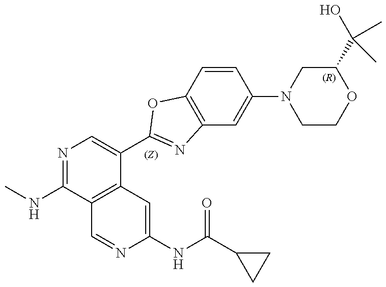 | LCMS (ESI) m/z 503.3 [M + H]⁺, ¹H NMR (400 MHz, CD₃OD) δ ppm 9.76 (s, 1H), 9.27 (s, 1H), 8.88 (s, 1H), 7.57 (d, J = 8.80 Hz, 1H), 7.41 (d, J = 2.32 Hz, 1H), 7.12 (dd, J = 8.86, 2.38 Hz, 1H), 4.11 (dd, J = 11.37, 2.20 Hz, 1H), 3.77- 3.90 (m, 1H), 3.68 (t, J = 5.69 Hz, 1H), 3.50 (dt, J = 10.70, 2.84 Hz, 2H), 3.17 (s, 3H), 2.79- 2.89 (m, 1H), 2.70 (t, J = 11.00 Hz, 1H), 1.96-2.05 (m, 1H), 1.30 (d, J = 9.78 Hz, 6H), 1.07-1.13 (m, 2H), 0.95-1.01 (m, 2H). |
| | (R)-N-(5-(5-(2-(2-hydroxypropan-2- | |
| | yl)morpholino)benzo[d]oxazol-2-yl)-8- | |
| | (methylamino)-2,7-naphthyridin-3- | |
| | yl)cyclopropanecarboxamide | |
| | Isomer obtained by chiral SFC purification | |
| | of example 924; the configuration has been | |
| | arbitrarily assigned. | |
| Example 926 | 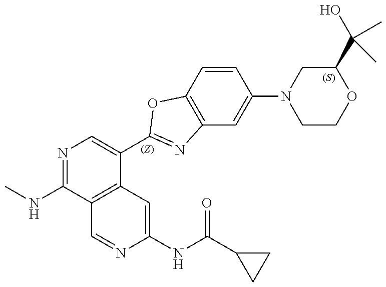 | LCMS (ESI) m/z 503.3 [M + H]⁺, ¹H NMR (400 MHz, CD₃OD) δ ppm 9.78 (s, 1H), 9.28 (s, 1H), 8.90 (s, 1H), 7.57 (d, J = 8.93 Hz, 1H), 7.41 (d, J = 2.20 Hz, 1H), 7.13 (dd, J = 8.86, 2.38 Hz, 1H), 4.11 (dd, J = 10.76, 2.20 Hz, 1H), 3.84 (td, J = 11.49, 2.20 Hz, 1H), 3.69 (br d, J = 11.49 Hz, 1H), 3.45- 3.54 (m, 2H), 3.18 (s, 3H), 2.85 (td, J = 11.89, 3.48 Hz, 1H), 2.70 (t, J = 10.94 Hz, 1H), 1.98- 2.05 (m, 1H), 1.30 (d, J = 9.78 Hz, 6H), 1.08-1.14 (m, 2H), 0.95-1.01 (m, 2H). |
| | (S)-N-(5-(5-(2-(2-hydroxypropan-2- | |
| | yl)morpholino)benzo[d]oxazol-2-yl)-8- | |
| | (methylamino)-2,7-naphthyridin-3- | |
| | yl)cyclopropanecarboxamide | |
| | Isomer obtained by chiral SFC purification | |
| | of example 924; the configuration has been | |
| | arbitrarily assigned. | |
| Example 927 | 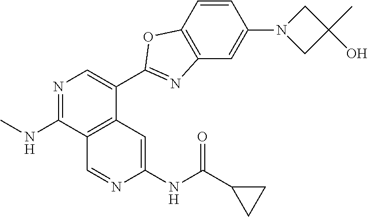 [SMILES: CNc1ncc(-c2nc3cc(N4CC(C)(O)C4)ccc3o2)c2cc(NC(=O)C3CC3)ncc12] | LCMS (ESI) m/z 445.4 [M + H]⁺; ¹H NMR (500 MHz, DMSO-d₆) δ ppm 11.15 (s, 1H), 9.68 (s, 1H), 9.47 (s, 1H), 9.08 (br s, 1H), 8.77 (s, 1H), 7.54 (d, J = 8.70 Hz, 1H), 6.74 (d, J = 1.98 Hz, 1H), 6.52 (dd, J = 8.70, 2.14 Hz, 1H), 3.83 (br d, J = 7.17 Hz, 4H), 3.11 (d, J = 4.27 Hz, 3H), 2.06-2.13 (m, 1H), 1.49 (s, 3H), 0.85-0.92 (m, 4H). |
| | N-(5-(5-(3-hydroxy-3-methylazetidin-1- | |
| | yl)benzo[d]oxazol-2-yl)-8- | |
| | (methylamino)-2,7-naphthyridin-3- | |
| | yl)cyclopropanecarboxamide | |
| Example 928 | 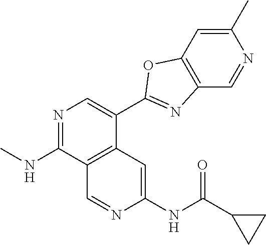 [SMILES: CNc1ncc(-c2nc3cnc(C)cc3o2)c2cc(NC(=O)C3CC3)ncc12] | LCMS (ESI) m/z 375.1 [M + H]⁺; ¹H NMR (400 MHz, DMSO-d₆) δ ppm 11.26 (br s, 1H), 10.16 (br s, 1H), 9.57- 9.83 (m, 2H), 9.43 (br s, 1H), 8.77 (br s, 1H), 8.31 (br s, 1H), 3.19 (br s, 3H), 2.85 (br s, 3H), 2.11 (br s, 1H), 0.91 (br s, 4H). |
| | N-(8-(methylamino)-5-(6- methyloxazolo[4,5-c]pyridin-2-yl)-2,7- | |
| | naphthyridin-3- | |
| | yl)cyclopropanecarboxamide | |
| Example 929 | 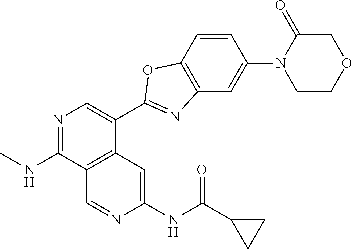 | LCMS (ESI) m/z 375.1 [M + H]⁺; ¹H NMR (400 MHz, DMSO-d₆) δ ppm 11.38 (s, 1H), 10.37 (br s, 1H), 9.60-9.92 (m, 2H), 8.59 (s, 1H), 7.75-7.96 (m, 2H), 7.47 (dd, J = 8.68, 2.08 Hz, 1H), 4.26 (s, 2H), 4.03- 4.09 (m, 2H), 3.85 (t, J = 4.95 Hz, 2H), 3.25 (s, 3H), 2.05- 2.20 (m, 1H), 0.87-1.03 (m, 4H). |
| | N-(8-(methylamino)-5-(5-(3- | |
| | oxomorpholino)benzo[d]oxazol-2-yl)-2,7- | |
| | naphthyridin-3- | |
| | yl)cyclopropanecarboxamide | |
| Example 930 | 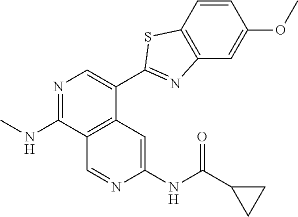 | LCMS (ESI) m/z 406.0 [M + H]⁺; ¹H NMR (400 MHz, CD₃OD) δ ppm 9.51 (s, 1H), 9.29 (s, 1H), 8.54 (s, 1H), 7.86 (d, J = 8.63 Hz, 1H), 7.67 (d, J = 1.88 Hz, 1H), 7.01-7.12 (m, 1H), 3.95 (s, 3H), 3.17 (s, 3H), 1.98 (br d, J = 4.00 Hz, 1H), 1.05 (br s, 2H), 0.92-0.98 (m, 2H). |
| | N-(5-(5-methoxybenzo[d]thiazol-2-yl)-8- | |
| | (methylamino)-2,7-naphthyridin-3- | |
| | yl)cyclopropanecarboxamide | |
| Example 931 | 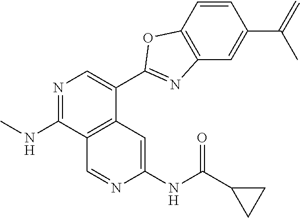 [SMILES: C=C(C)c1ccc2oc(-c3cnc(NC)c4cnc(NC(=O)C5CC5)cc34)nc2c1] | LCMS (ESI) m/z 400.2 [M + H]⁺; ¹H NMR (400 MHz, CD₃OD) δ ppm 10.05 (s, 1H), 9.49 (s, 1H), 8.49 (s, 1H), 7.98- 8.05 (m, 1H), 7.61-7.67 (m, 2H), 5.50 (s, 1H), 5.21 (s, 1H), 2.28 (s, 3H), 2.02-2.06 (m, 1H), 1.65 (s, 3H), 1.14-1.17 (m, 2H), 1.02-1.06 (m, 2H). |
| | N-(8-(methylamino)-5-(5-(prop-1-en-2- | |
| | yl)benzo[d]oxazol-2-yl)-2,7- | |
| | naphthyridin-3- | |
| | yl)cyclopropanecarboxamide | |
| Example 932 | 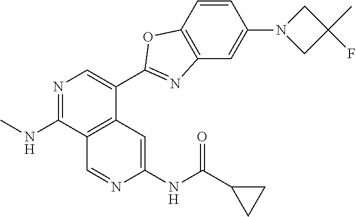 [SMILES: CNc1ncc(-c2nc3cc(N4CC(C)(F)C4)ccc3o2)c2cc(NC(=O)C3CC3)ncc12] | LCMS (ESI) m/z 447.3 [M + H]⁺; ¹H NMR (400 MHz, CH₃OD) δ ppm 9.73 (s, 1H), 9.25 (s, 1H), 8.87 (s, 1H), 7.52 (d, J = 8.6 Hz, 1H), 6.92 (d, J = 2.1 Hz, 1H), 6.59 (dd, J = 8.7, 2.3 Hz, 1H), 4.01 (s, 2H), 3.97 (s, 2H), 3.16 (s, 3H), 1.95-2.05 (m, 1H), 1.66-1.76 (m, 3H), 1.06-1.14 (m, 2H), 0.97 (dq, J = 7.5, 3.7 Hz, 2H). |
| | N-(5-(5-(3-fluoro-3-methylazetidin-1- | |
| | yl)benzo[d]oxazol-2-yl)-8- | |
| | (methylamino)-2,7-naphthyridin-3- | |
| | yl)cyclopropanecarboxamide: | |
| Example 933 | 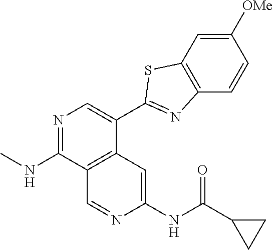 [SMILES: CNc1ncc(-c2nc3ccc(OC)cc3s2)c2cc(NC(=O)C3CC3)ncc12] | LCMS (ESI) m/z 406.1 [M + H]⁺; ¹H NMR (400 MHz, DMSO-d₆) δ ppm 11.04 (s, 1H), 9.53 (s, 1H), 9.44 (s, 1H), 8.51- 8.55 (m, 2 H), 7.88 (d, J = 8.88 Hz, 1H), 7.71 (d, J = 2.50 Hz, 1H), 7.14 (dd, J = 8.94, 2.56 Hz, 1H), 3.86 (s, 3H), 3.06 (d, J = 4.25 Hz, 3H), 2.06-2.10 (m, 1H), 0.82-0.87 (m, 5H). |
| | N-(5-(6-methoxybenzo[d]thiazol-2-yl)-8- | |
| | (methylamino)-2,7-naphthyridin-3- | |
| | yl)cyclopropanecarboxamide: | |
| Example 934 | 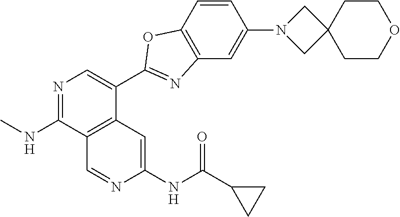 [SMILES: CNc1ncc(-c2nc3cc(N4CC5(CCOCC5)C4)ccc3o2)c2cc(NC(=O)C3CC3)ncc12] | LCMS (ESI) m/z 485.3 [M + H]+; ¹H NMR (400 MHz, CD₃OD) δ ppm 9.73 (s, 1H), 9.25 (s, 1H), 8.87 (s, 1H), 7.52 (d, J = 8.6 Hz, 1H), 6.92 (d, J = 2.1 Hz, 1H), 6.59 (dd, J = 8.7, 2.3 Hz, 1H), 3.68-3.79 (m, 8 H), 3.16 (s, 3H), 1.95-2.05 (m, 1H), 1.84-1.92 (m, 4H), 1.06- 1.14 (m, 2H), 0.97 (br dd, J = 7.6, 3.2 Hz, 2 H). |
| | N-(5-(5-(7-oxa-2-azaspiro[3.5]nonan-2- | |
| | yl)benzo[d]oxazol-2-yl)-8- | |
| | (methylamino)-2,7-napththyridin-3- | |
| | yl)cyclopropanecarboxamide | |
| Example 935 | 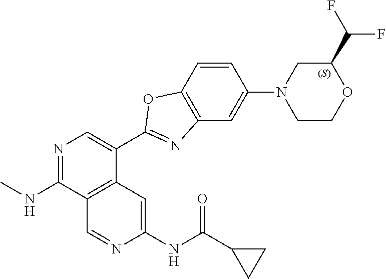 (S)-N-(5-(5-2- (difluoromethyl)morpholino)benzo[d]oxa- | LCMS (ESI) m/z 495.2 [M + H]⁺, ¹H NMR (400 MHz, DMSO-d₆) δ ppm 11.05 (s, 1 H), 9.66 (s, 1 H), 9.44 (s, 1 H), 8.89 (s, 1 H), 8.64 (br d, J = 4.62 Hz, 1H), 7.61 (d, J = 8.80 Hz, 1H), 7.28 (d, J = 2.20 Hz, 1H), 7.10 (dd, J = 8.91, 2.31 Hz, 1H), 5.95-6.35 (m, 1H), 4.06 (br d, J = 10.78 Hz, 1H), 3.96 (br d, J = 10.78 Hz, 1H), 3.77 (td, J = 11.28, 2.31 Hz, 1H), 3.64 (br d, J = 10.78 Hz, 1H), 3.55 (br d, J = 12.32 Hz, 1H), 3.09 (d, J = 3.96 Hz, 3H), 2.82 (td, J = 11.61, 3.19 Hz, 1H), 2.68-2.76 (m, 1H), 2.06- 2.14 (m, 1H), 0.84-0.95 (m, 4H). |
| | zol-2-yl)-8-(methylamino)-2,- | |
| | naphthyridin-3- | |
| | yl)cyclopropanecarboxamide | |
| | Isomer obtained by chiral SFC purification | |
| | of example 329; the configuration has been | |
| | arbitrarily assigned. | |
| Example 936 | 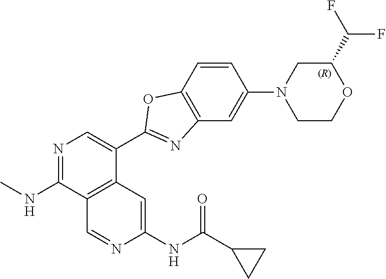 | LCMS (ESI) m/z 495.2 [M + H]⁺, ¹H NMR (400 MHz, DMSO-d₆) δ ppm 11.05 (s, 1H), 9.66 (s, 1H), 9.45 (s, 1H), 8.89 (s, 1H), 8.66 (br d, J = 4.62 Hz, 1H), 7.61 (d, J = 8.80 Hz, 1H), 7.28 (d, J = 2.20 Hz, 1H), 7.10 (dd, J = 8.91, 2.09 Hz, 1H), 5.98-6.34 (m, 1H), 4.03-4.10 (m, 1H), 3.91-4.01 (m, 1H), 3.72-3.82 (m, 1H), 3.64 (br d, J = 10.78 Hz, 1H), 3.56 (br d, J = 11.66 Hz, 1H), 3.09 (d, J = 3.96 Hz, 3H), 2.82 (td, J = 11.66, 2.86 Hz, 1H), 2.68-2.75 (m, 1 H), 2.04-2.19 (m, 1 H), 0.84-0.97 (m, 4 H). |
| | (R)-N-(5-(5-(2- | |
| | (difluoromethyl)morpholino)benzo[d]oxa- | |
| | zol-2-yl)-8-(methylamino)-2,7- | |
| | naphthyridin-3- | |
| | yl)cyclopropanecarboxamide | |
| | Isomer obtained by chiral SFC purification | |
| | of example 329; the configuration has been | |
| | arbitrarily assigned. | |
| Example 937 | 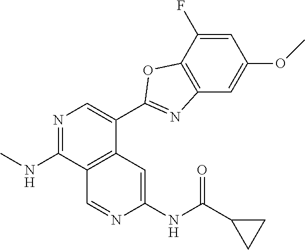 | LCMS (ESI) m/z 408.0 [M + H]⁺; ¹H NMR (400 MHz, CD₃OD) δ ppm 9.79 (s, 1H), 9.24 (s, 1H), 8.89 (s, 1H), 7.21 (d, J = 2.0 Hz, 1H), 6.77 (dd, J = 11.8, 2.0 Hz, 1H), 3.16 (s, 3H), 3.89 (s, 3H), 2.02-1.95 (m, 1H), 1.11-1.06 (m, 2H), 0.98-0.93 (m, 4H). |
| | N-(5-(7-fluoro-5- | |
| | methoxybenzo[d]oxazol-2-yl)-8- | |
| | (methylamino)-2,7-naphthyridin-3- | |
| | yl)cyclopropanecarboxamide | |
| Example 938 | 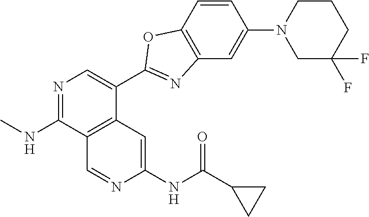 [SMILES: CNc1ncc(-c2nc3cc(N4CCCC(F)(F)C4)ccc3o2)c2cc(NC(=O)C3CC3)ncc12] | LCMS (ESI) m/z 479.1 [M + H]⁺; ¹H NMR (400 MHz, DMSO-d₆) δ ppm 11.05 (s, 1H), 9.63 (s, 1H), 9.43 (s, 1H), 8.88 (s, 1H), 8.64 (br d, J = 4.40 Hz, 1H), 7.57 (d, J = 8.80 Hz, 1H), 7.27 (d, J = 2.20 Hz, 1H), 7.08 (dd, J = 8.91, 2.53 Hz, 1H), 3.47 (br t, J = 11.77 Hz, 2H), 3.24 (br s, 2H), 3.08 (d, J = 4.40 Hz, 3H), 2.16-1.99 (m, 3H), 1.84 (br s, 2H), 0.94-0.82 (m, 4H). |
| | N-[5-[5-(3,3-difluoro-1-piperidyl)-1,3- | |
| | benzoxazol-2-yl]-8-(methylamino)-2,7- | |
| | naphthyridin-3- | |
| | yl]cyclopropanecarboxamide | |
| Example 939 | 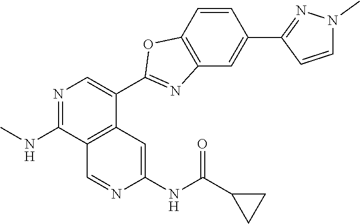 | LCMS (ESI) m/z 440.1 [M + H]⁺; ¹H NMR (400 MHz, DMSO-d₆) δ ppm 11.06 (s, 1H), 9.74 (s, 1H), 9.44 (s, 1H), 8.92 (s, 1H), 8.67 (br d, J = 4.3 Hz, 1H), 8.10 (s, 1H), 7.84 (dd, J = 8.5, 1.5 Hz, 1H), 7.76 (d, J = 2.3 Hz, 1H), 7.72 (d, J = 8.5 Hz, 1H), 6.82 (d, J = 2.0 Hz, 1H), 3.91 (s, 3H), 3.09 (d, J = 4.3 Hz, 3H), 2.10 (td, J = 8.0, 4.0 Hz, 1H), 0.93 (br d, J = 4.0 Hz, 2H), 0.83-0.89 (m, 2H). |
| | N-(5-(5-(1-methyl-1H-pyrazol-3- | |
| | yl)benzo[d]oxazol-2-yl)-8- | |
| | (methylamino)-2,7-naphthyridin-3- | |
| | yl)cyclopropanecarboxamide | |
| Example 940 | 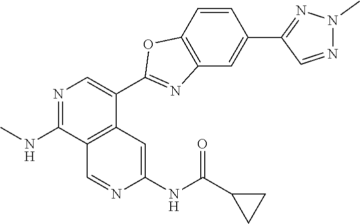 | LCMS (ESI) m/z 441.0 [M + H]⁺; ¹H NMR (400 MHz, DMSO-d₆) δ ppm 11.07 (s, 1H), 9.73 (s, 1H), 9.44 (s, 1H), 8.94 (s, 1H), 8.67-8.74 (m, 1H), 8.37 (s, 1H), 8.18 (d, J = 1.3 Hz, 1H) ,7.90-7.86 (m, 1H), 7.83-7.79 (m, 1H), 4.22 (s, 3H), 3.09 (d, J = 4.3 Hz, 3H), 2.14-2.07 (m, 1H), 0.96-0.90 (m, 2H), 0.87 (dt, J = 7.8, 2.9 Hz, 2H). |
| | N-(5-(5-(2-methyl-2H-1,2,3-triazol-4- | |
| | yl)benzo[d]oxazol-2-yl)-8- | |
| | (methylamino)-2,7-naphthyridin-3- | |
| | yl)cyclopropanecarboxamide | |
| Example 941 | 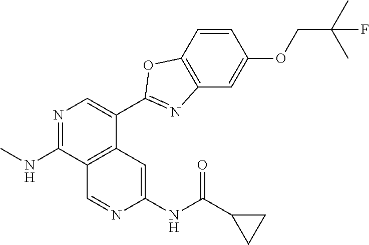 [SMILES: CNc1ncc(-c2nc3cc(OCC(C)(C)F)ccc3o2)c2cc(NC(=O)C3CC3)ncc12] | LCMS (ESI) m/z 450.1 [M + H]⁺; ¹H NMR (400 MHz, DMSO-d₆) δ ppm 11.04 (s, 1H), 9.66 (s, 1H), 9.43 (s, 1H), 8.89 (s, 1H), 8.66 (br d, J = 4.5 Hz, 1H), 7.62 (d, J = 8.9 Hz, 1H), 7.32 (d, J = 2.4 Hz, 1H), 7.00 (dd, J = 8.8, 2.4 Hz, 1H), 4.16- 4.08 (m, 2H), 3.08 (d, J = 4.3 Hz, 3H), 2.16-2.04 (m, 1H), 1.49 (s, 3H), 1.44 (s, 3H), 0.94- 0.83 (m, 4H). |
| | N-[5-[5-(2-fluoro-2-methyl-propoxy)-1,3- | |
| | benzoxazol-2-yl]-8-(methylamino)-2,7- | |
| | naphthyridin-3- | |
| | yl]cyclopropanecarboxamide | |
| Example 942 | 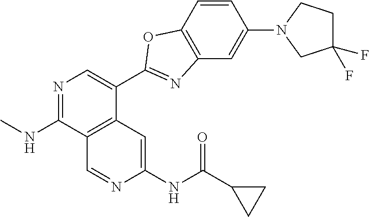 [SMILES: CNc1ncc(-c2nc3cc(N4CCC(F)(F)C4)ccc3o2)c2cc(NC(=O)C3CC3)ncc12] | LCMS (ESI) m/z 465.1 [M + H]⁺, ¹H NMR (400 MHz, DMSO-d₆) δ ppm 11.03 (s, 1H), 9.64 (s, 1H), 9.43 (s, 1H), 8.88 (s, 1H), 8.62 (br d, J = 4.27 Hz, 1H), 7.57 (d, J = 8.78 Hz, 1H), 6.91 (d, J = 2.26 Hz, 1H), 6.70 (dd, J = 8.78, 2.26 Hz, 1H), 3.77 (t, J = 13.30 Hz, 2H), 3.55 (t, J = 7.28 Hz, 2H), 3.08 (d, J = 4.02 Hz, 3H), 2.54-2.61 (m, 2H), 2.05-2.14 (m, 1H), 0.82- 0.92 (m, 4H). |
| | N-(5-(5-(3,3-difluoropyrrolidin-1- | |
| | yl)benzo[d]oxazol-2-yl)-8- | |
| | (methylamino)-2,7-naphthyridin-3- | |
| | yl)cyclopropanecarboxamide | |
| Example 943 | 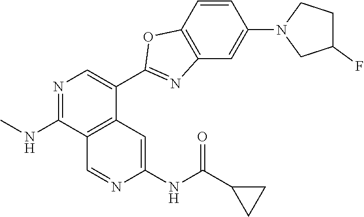 [SMILES: CNc1ncc(-c2nc3cc(N4CCC(F)C4)ccc3o2)c2cc(NC(=O)C3CC3)ncc12] N-(5-(5-(3-fluoropyrrolidin-1- | LCMS (ESI) m/z 446.9 [M + H]+; 1H NMR (400 MHz, DMSO-d6) δ ppm 11.03 (s, 1H), 9.66 (s, 1H), 9.42 (s, 1H), 8.88 (s, 1H), 8.60 (br d, J = 4.4 Hz, 1H), 7.54 (d, J = 8.8 Hz, 1H), 6.83 (d, J = 2.0 Hz, 1H), 6.64 (dd, J = 8.8, 2.4 Hz, 1H), 5.39-5.57 (m, 1H), 3.49-3.69 (m, 2H), 3.40-3.48 (m, 2H), 3.07 (d, J = 4.4 Hz, 3H), 2.28- 2.35 (m, 1H), 2.17-2.26 (m, 1H), 2.06-2.14 (m, 1H), 0.89- 0.96 (m, 2H), 0.80-0.88 (m, |
| | yl)benzo[d]oxazol-2-yl)-8-(methylamino)- | 2H). |
| | 2,7-naphthyridin-3- | |
| | yl)cyclopropanecarboxamide | |
| Example 944 | 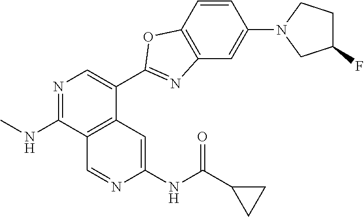 [SMILES: CNc1ncc(-c2nc3cc(N4CC[C@@H](F)C4)ccc3o2)c2cc(NC(=O)C3CC3)ncc12] (R)-N-(5-(5-(3-fluoropyrrolidin-1- yl)benzo[d]oxazol-2-yl)-8-(methylamino)- | LCMS (ESI) m/z 446.9 [M + H]+; Chiral purity: 97.8%; 1H NMR (400 MHz, DMSO- d6) δ ppm 11.03 (s, 1H), 9.66 (s, 1H), 9.42 (s, 1H), 8.88 (s, 1H), 8.60 (br d, J = 4.4 Hz, 1H), 7.54 (d, J = 8.8 Hz, 1H), 6.83 (d, J = 2.0 Hz, 1H), 6.64 (dd, J = 8.8, 2.4 Hz, 1H), 5.39-5.57 (m, 1H), 3.49-3.69 (m, 2H), 3.40-3.48 (m, 2H), 3.07 (d, J = 4.4 Hz, 3H), 2.28-2.35 (m, 1H), 2.17-2.26 (m, 1H), 2.06- 2.14 (m, 1H), 0.89-0.96 (m, 2H), 0.80-0.88 (m, 2H). |
| | 2,7-naphthyridin-3- | |
| | yl)cyclopropanecarboxamide | |
| | Isomer obtained by chiral SFC purification | |
| | of example 943; the configuration has been | |
| | arbitrarily assigned. | |
| Example 945 | 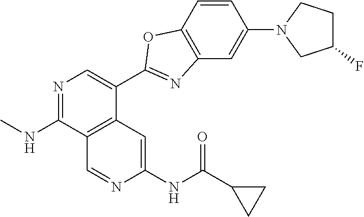 [SMILES: CNc1ncc(-c2nc3cc(N4CC[C@H](F)C4)ccc3o2)c2cc(NC(=O)C3CC3)ncc12] (S)-N-(5-(5-(3-fluoropyrrolidin-1- yl)benzo[d]oxazol-2-yl)-8-(methylamino)- | LCMS (ESI) m/z 446.9 [M + H]+; Chiral purity: 90.3%; 1H NMR (400 MHz, DMSO- d6) δ ppm 11.03 (s, 1H), 9.66 (s, 1H), 9.42 (s, 1H), 8.88 (s, 1H), 8.60 (br d, J = 4.4 Hz, 1H), 7.54 (d, J = 8.8 Hz, 1H), 6.83 (d, J = 2.0 Hz, 1H), 6.64 (dd, J = 8.8, 2.4 Hz, 1H), 5.39-5.57 (m, 1H), 3.49-3.69 (m, 2H), 3.40-3.48 (m, 2H), 3.07 (d, J = 4.4 Hz, 3H), 2.28-2.35 (m, 1H), 2.17-2.26 (m, 1H), 2.06- 2.14 (m, 1H), 0.89-0.96 (m, 2H), 0.80-0.88 (m, 2H). |
| | 2,7-naphthyridin-3- | |
| | yl)cyclopropanecarboxamide | |
| | Isomer obtained by chiral SFC purification | |
| | of example 943; the configuration has been | |
| | arbitrarily assigned. | |
| Example 946 | 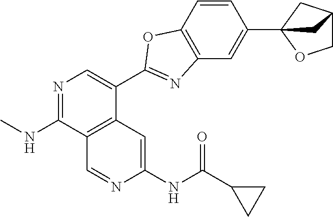 [SMILES: CNc1ncc(-c2nc3cc([C@]45C[C@H](CO4)C5)ccc3o2)c2cc(NC(=O)C3CC3)ncc12] | LCMS (ESI) m/z 442.0 [M + H]⁺; ¹H NMR (400 MHz, DMSO-d₆) δ ppm 11.05 (s, 1H), 9.72 (s, 1H), 9.44 (s, 1H), 8.91 (s, 1H), 8.68 (br d, J = 4.4 Hz, 1H), 7.68-7.76 (m, 2H), 7.44 (dd, J = 8.0, 1.6 Hz, 1H), 3.84 (s, 2 H), 3.08 (d, J = 4.0 Hz, 3H), 3.00 (t, J = 3.3 Hz, 1H), 2.20 (br s, 2H), 2.08-2.12 (m, 1H), 1.72 (dd, J = 4.3, 1.6 Hz, 2H), 0.92 (br d, J = 4.0 Hz, 2H), 0.84-0.88 (m, 2H). |
| | N-(5-(5-((1s,4s)-2-oxabicyclo[2.1.1]hexan- | |
| | 1-yl)benzo[d]oxazol-2-yl)-8- | |
| | (methylamino)-2,7-naphthyridin-3- | |
| | yl)cyclopropanecarboxamide | |

### Example 947: Synthesis of N-(8-(methylamino)-5-(5-(2-methyltetrahydrofuran-2-yl)benzo[d]oxazol-2-yl)-2,7-naphthyridin-3-yl)cyclopropanecarboxamide

### Step 1. Synthesis of 5-(2-methyltetrahydrofuran-2-yl)-1,3-benzoxazole

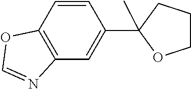

A mixture of 5-bromo-1,3-benzoxazole (100 mg, 0.51 mmol, 1 eq), 2-methyltetrahydrofuran-2-carboxylic acid (98.6 mg, 0.76 mmol, 1.5 eq), Ir[dF(CF₃)ppy]₂(dtbpy)(PF₆) (28.3 mg, 0.025 mmol, 0.05 eq), 4-methoxy-2-(4-methoxy-2-pyridyl)pyridine (10.9 mg, 0.05 mmol, 0.1 eq), NiCl₂.glyme (11.1 mg, 0.05 mmol, 0.1 eq) and phthalimide (74.3 mg, 0.51 mmol, 1 eq) and BTMG (260 mg, 1.52 mmol, 3 eq) in DMSO (2 mL) was degassed and purged with N₂ for 3 times, and then the mixture was stirred at 25° C. for 72 hr under N₂ atmosphere and irradiated with two 34W blue LEDs. The reaction mixture was diluted with H₂O (30 mL×2) and extracted with EtOAc (20 mL×2), dried over Na₂SO₄, concentrated under reduced pressure to give a residue. The residue was purified by column chromatography (ISCO®; 12 g SepaFlash® Silica Flash Column, petroleum ether/EtOAc with EtOAc from 0-10%, 30 mL/min, 254 nm) to afford 5-(2-methyltetrahydrofuran-2-yl)-1,3-benzoxazole (38 mg, 12.2% yields) as a colorless oil. LCMS (ESI) m/z 204.1 [M+H]⁺.

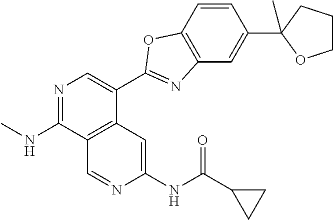 [SMILES: CNc1ncc(-c2nc3cc(C4(C)CCCO4)ccc3o2)c2cc(NC(=O)C3CC3)ncc12]

Step 2. Synthesis of N-(8-(methylamino)-5-(5-(2-methyltetrahydrofuran-2-yl)benzo[d]oxazol-2-yl)-2,7-naphthyridin-3-yl)cyclopropanecarboxamide was synthesized using a similar procedure that was previously described in example 1 by using N-(5-bromo-8-(methylamino)-2,7-naphthyridin-3-yl)cyclopropanecarboxamide and 5-(3,3-difluoropyrrolidin-1-yl)benzo[d]oxazole as the starting material. LCMS (ESI) m/z 444.3 [M+H]⁺, ¹H NMR (400 MHz, DMSO-d₆) δ ppm 11.06 (s, 1H), 9.70 (s, 1H), 9.43 (s, 1H), 8.90 (s, 1H), 8.65 (br d, J=4.8 Hz, 1H), 7.72 (s, 1H), 7.65 (br d, J=8.4 Hz, 1H), 7.41 (br d, J=8.0 Hz, 1H), 3.83-3.99 (m, 2H), 3.08 (br d, J=3.6 Hz, 3H), 2.1.5-2.24 (m, 1H), 2.04-2.13 (m, 2H), 1.92-2.02 (m, 1H), 1.67-1.82 (m, 1H), 1.51 (s, 3H), 0.81-0.95 (m, 4H).

### Example 948 and 949: Synthesis of (S)—N-(8-(methylamino)-5-(5-(2-methyltetrahydrofuran-2-yl)benzo[d]oxazol-2-yl)-2,7-naphthyridin-3-yl)cyclopropanecarboxamide (Example 948) and synthesis of (R)—N-(8-(methylamino)-5-(5-(2-methyltetrahydrofuran-2-yl)benzo[d]oxazol-2-yl)-2,7-naphthyridin-3-yl)cyclopropanecarboxamide (Example 949)

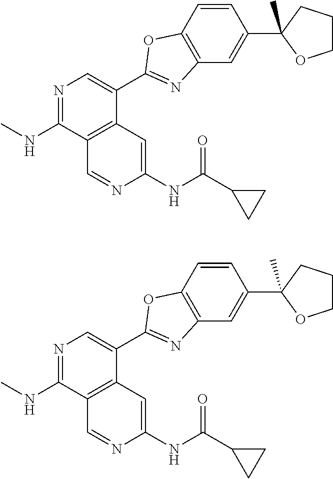 [SMILES: CNc1ncc(-c2nc3cc([C@]4(C)CCCO4)ccc3o2)c2cc(NC(=O)C3CC3)ncc12 ; CNc1ncc(-c2nc3cc([C@@]4(C)CCCO4)ccc3o2)c2cc(NC(=O)C3CC3)ncc12]

| Example | Isomers | Characterization data |
|---|---|---|
| Example 947 | The racemic mixture was | FR-a: LCMS (ESI) m/z 444.3 [M + H]⁺ , |
| Example 948 | separated by chiral SFC using | ¹H NMR (400 MHz, DMSO-d₆) δ ppm |
| Example 949 | Example 947 and the two | 11.05 (s, 1H), 9.70 (s, 1H), 9.43 (s, 1H), |
| | configurations are arbitrarily | 8.90 (s, 1H), 8.65 (br d, J = 4.4 Hz, 1H), |
| | assigned | 7.72 (d, J = 1.2 Hz, 1H), 7.65 (d, J = 8.4 |
| | | Hz, 1H), 7.41 (dd, J = 8.4, 1.6 Hz, 1H), |
| | | 3.96 (q, J = 7.2 Hz, 1H), 3.87 (td, J = |
| | | 8.0, 5.6 Hz, 1H), 3.08 (d, J = 4.4 Hz, |
| | | 3H), 2.15-2.24 (m, 1H), 2.04-2.14 (m, |
| | | 2H), 1.91-2.03 (m, 1H), 1.69-1.81 (m, |
| | | 1H), 1.51 (s, 3H), 0.89-0.96 (m, 2H), |
| | | 0.81-0.88 (m, 2H). |
| | | FR-b: LCMS (ESI) m/z 444.3 [M + H]⁺, |
| | | ¹H NMR (400 MHz, DMSO-d₆) δ ppm |
| | | 11.05 (s, 1H), 9.70 (s, 1H), 9.43 (s, 1H), |
| | | 8.90 (s, 1H), 8.65 (br d, J = 4.4 Hz, 1H), |
| | | 7.72 (d, J = 1.2 Hz, 1H), 7.65 (d, = 8.4 |
| | | Hz, 1H), 7.41 (dd, J = 8.4, 1.6 Hz, 1H), |
| | | 3.92-4.00 (m, 1H), 3.87 (td, J = 8.0, 6.0 |
| | | Hz, 1H), 3.08 (d, J = 4.4 Hz, 3H), 2.15- |
| | | 2.24 (m, 1H), 2.04-2.14 (m, 2H), 1.92- |
| | | 2.03 (m, 1H), 1.70-1.82 (m, 1H), 1.51 |
| | | (s, 3H), 0.89-0,95 (m, 2H), 0.82-0.89 |
| | | (m, 2H). |

### Example 950

### Synthesis of N-(8-(methylamino)-5-(pyrimidin-2-ylethynyl)-2,7-naphthyridin-3-yl)cyclopropanecarboxamide

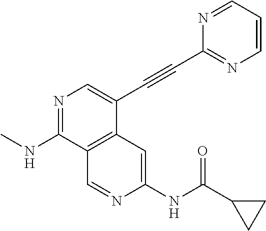 [SMILES: CNc1ncc(C#Cc2ncccn2)c2cc(NC(=O)C3CC3)ncc12]

A mixture of N-[5-ethynyl-8-(methylamino)-2,7-naphthyridin-3-yl]cyclopropanecarboxamide (100 mg, 375 μmol), 2-chloropyrimidine (43.0 mg, 375 μmol), Et₃N (114 mg, 1.1.3 mmol), XPhos (35.8 mg, 75.1 μmol), XPhos Pd G₃ (63.5 mg, 75.1 μmol) and CuI (14.3 mg, 75.1 μmol) in DMF (4 mL) was stirred at 90° C. for 1 hr under N₂ atmosphere. The solution was added H₂O (20 mL), diluted EtOAc (20 mL), filtered and extracted with EtOAc (20 mL×2) and concentrated to give a residue. The residue was purified by flash chromatography (ISCO®; 4 g SepaFlash® Silica Flash Column, petroleum ether/EtOAc with EtOAc from 0-100%, 12 mL/min, 254 mn), to provide N-[8-(methylamino)-5-(2-pyrimidin-2-ylethynyl)-2,7-naphthyridin-3-yl]cyclopropanecarboxamide (56.1 mg, 43.4% yield) as a yellow solid. LCMS (ESI) m/z 345.4 [M+H]+; 1H NMR (400 MHz, DMSO-d₆) δ ppm 11.13 (s, 1H), 9.41 (s, 1H), 8.85 (d, J=4.9 Hz, 2H), 8.58 (s, 2H), 8.41 (s, 1H), 7.48 (t, J=5.0 Hz, 1H), 3.04 (d, J=4.3 Hz, 3H),2.08 (brs, 1H), 0.81-0.90 (m, 4H).

### Examples 951-1021

Each compound in Table 31 (below) was prepared using a similar experimental procedure to prepare Example 950 using N-(5-ethynyl-8-(methylamino)-2,7-naphthyridin-3-yl)cyclopropanecarboxamide as the common intermediate and appropriate aryl halides.

TABLE 31

| Example | Structure and Name | MS and ¹H NMR |
|---|---|---|
| Example 951 | 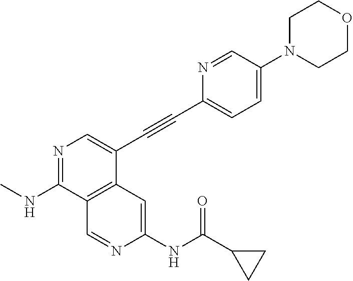 [SMILES: CNc1ncc(C#Cc2ccc(N3CCOCC3)cn2)c2cc(NC(=O)C3CC3)ncc12] | LCMS (ESI) m/z 429.1 [M + H]⁺; ¹H NMR (400 MHz, DMSO-d₆) δ ppm 11.10 (s, 1H), 9.38 (s, 1H), 8.68 (s, 1H), 8.04-8.45 (m, 3H), 7.44- 7.51 (m, 1H), 7.34-7.41 (m, 1H), 3.73-3.80 (m, 4H), 3.24- 3.29 (m, 4H), 2.98-3.05 (m, 3H), 2.01-2.13 (m, 1H), 0.82- 0.91 (m, 4H). |
| | N-(8-(methylamino)-5-((5- | |
| | morpholinopyridin-2-yl)ethynyl)-2,7- | |
| | naphthyridin-3- | |
| | yl)cyclopropanecarboxamide | |
| Example 952 | 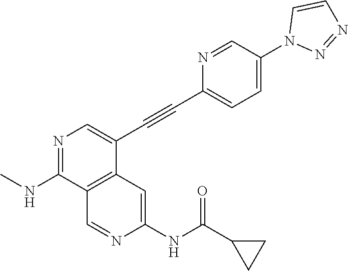 [SMILES: CNc1ncc(C#Cc2ccc(-n3ccnn3)cn2)c2cc(NC(=O)C3CC3)ncc12] | LCMS (ESI) m/z 411.3 [M + H]⁺; ¹H NMR (400 MHz, DMSO-d₆) δ ppm 10.89 (s, 1H), 9.35 (s, 1H), 9.14 (s, 1H), 8.92 (s, 1H), 8.6s (s, 1H), 8.51 (br d, J = 4.88 Hz, 1H), 8.42-8.47 (m, 1H), 8.30- 8.42 (m, 1H), 8.07 (s, 1H), 7.83-7.89 (m, 1H), 3.03 (s, 3H), 2.06-2.12 (m, 1H), 0.84- 0.91 (m, 4H). |
| | N-(5-((5-(1H-1,2,3-triazol-1-yl)pyridin-2- | |
| | yl)ethynyl)-8-(methylamino)-2,7- | |
| | naphthyridin-3- | |
| | yl)cyclopropanecarboxamide | |
| Example 953 | 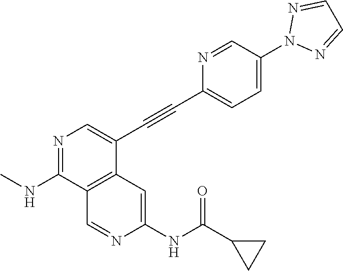 [SMILES: CNc1ncc(C#Cc2ccc(-n3nccn3)cn2)c2cc(NC(=O)C3CC3)ncc12] | LCMS (ESI) m/z 411.2 [M + H]⁺; ¹H NMR (400 MHz, DMSO-d₆) δ ppm 11.13 (s, 1H), 9.41 (s, 1H), 9.27 (s, 1H), 8.64 (s, 1H), 8.50-8.55 (m, 1H), 8.43-8.48 (m, 1H), 8.38-8.43 (m, 1H), 8.16- 8.38 (m, 2H), 7.71-7.87 (m, 1H), 3.04 (s, 3H), 2.07-2.14 (m, 1H), 0.89-0.93 (m, 2H), 0.84-0.87 (m, 2H). |
| | N-(5-((5-(2H-1,2,3-triazol-2-yl)pyridin-2- | |
| | yl)ethynyl)-8-(methylamino)-2,7- | |
| | naphthyridin-3- | |
| | yl)cyclopropanecarboxamide | |
| Example 954 | 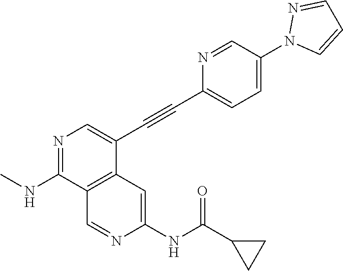 [SMILES: CNc1ncc(C#Cc2ccc(-n3cccn3)cn2)c2cc(NC(=O)C3CC3)ncc12] | LCMS (ESI) m/z 410.0 [M + H]⁺; ¹H NMR (400 MHz, DMSO-d₆) δ ppm 10.56 (s, 1H), 9.39 (s, 1H), 9.15 (s, 1H), 8.65-8.70 (m, 2H), 8.42- 8.50 (m, 1H), 8.36-8.40 (m, 1H), 8.31-8.35 (m, 1H), 7.85 (s, 1H), 7.69-7.82 (m, 1H), 6.64 (s, 1H), 3.03 (s, 3H), 2.06- 2.12 (m, 1H), 0.84-0.92 (m, 4H). |
| | N-(5-((5-(1H-pyrazol-1-yl)pyridin-2- | |
| | yl)ethynyl)-8-(methylamino)-2,7- | |
| | naphthyridin-3- | |
| | yl)cyclopropanecarboxamide | |
| Example 955 | 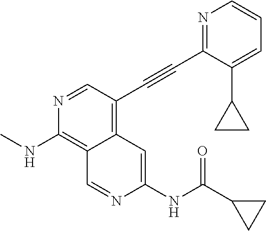 [SMILES: CNc1ncc(C#Cc2ncccc2C2CC2)c2cc(NC(=O)C3CC3)ncc12] | LCMS (ESI) m/z 384.2 [M + H]⁺; ¹H NMR (400 MHz, CD₃OD) δ ppm 9.18 (s, 1H), 8.66 (s, 1H), 8.33 (s, 1H), 8.26- 8.32 (m, 1H), 7.35-7.40 (m, 1H), 7.24-7.31 (m, 1H), 3.09 (m, 3 H), 2.69-2.76 (m, 1H), 1.88-1.95 (m, 1H), 1.26- 1.31 (m, 2H), 0.95-1.00 (m, 2H), 0.89-0.93 (m, 2H), 0.79 (m, 2H). |
| | N-(5-((3-cyclopropylpyridin-2-yl)ethynyl)-8- | |
| | (methylamino)-2,7-naphthyridin-3- | |
| | yl)cyclopropanecarboxamide | |
| Example 956 | 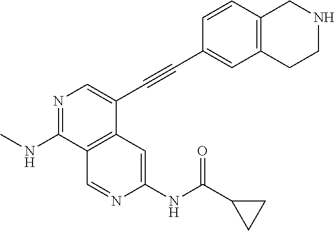 [SMILES: CNc1ncc(C#Cc2ccc3c(c2)CCNC3)c2cc(NC(=O)C3CC3)ncc12] | LCMS (ESI) m/z 398.4 [M + H]⁺; ¹H NMR (400 MHz, DMSO-d₆) δ ppm 11.27 (br s, 1H), 9.55 (br s, 3H), 8.74 (s, 1H), 8.19 (s, 1H), 7.52 (s, 1H), 7.46 (br d, J = 7.3 Hz, 1H), 7.32 (br d, J = 8.3 Hz, 1H), 4.30 (s, 2H), 3.28 (s, 3H), 3.07-3.09 (m, 4H), 2.11 (m, 1H), 0.85-0.96 (m, 4H). |
| | N-(8-(methylamino)-5-((1,2,3,4- | |
| | tetrahydroisoquinolin-6-yl)ethynyl)-2,7- | |
| | naphthyridin-3- | |
| | yl)cyclopropanecarboxamide | |
| Example 957 | 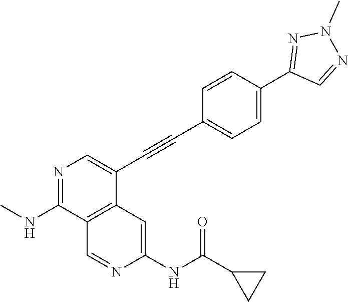 [SMILES: CNc1ncc(C#Cc2ccc(-c3cnn(C)n3)cc2)c2cc(NC(=O)C3CC3)ncc12] | LCMS (ESI) m/z 424.1 [M + H]⁺; ¹H NMR (400 MHz, DMSO-d₆) δ ppm 11.11 (s, 1H), 9.37 (s, 1H), 8.63 (s, 1H), 8.36-8.41 (m, 1H), 8.27- 8.34 (m, 2H), 7.89-7.95 (m, 2H), 7.62-7.66 (m, 2H), 4.19 (s, 3H), 3.05 (s, 3H), 2.04- 2.14 (m, 1H), 0.84-0.93 (m, 4 H). |
| | N-(5-((4-(2-methyl-2H-1,2,3-triazol-4- | |
| | yl)phenyl)ethynyl)-8-(methylamino)-2,7- | |
| | naphthyridin-3-yl)cyclopropanecarboxamide: | |
| Example 958 | 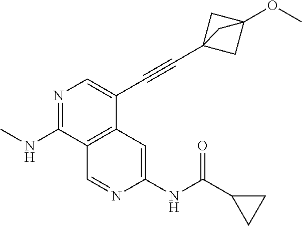 | LCMS (ESI) m/z 363.4 [M + H]⁺; ¹H NMR (400 MHz, CD₃OD) δ ppm 9.18 (s, 1H), 8.53 (s, 1H), 8.05 (s, 1H), 3.30 (s, 3H), 3.08 (s, 3H), 2.29 (s, 6H), 1.93-2.00 (m, 1H), 1.05- 1.09 (m, 2H), 0.95 (m, 2H). |
| | N-(5-((3-methoxybicyclo[1.1.1]pentan-1- | |
| | yl)ethynyl)-8-(methylamino)-2,7-naphthyridin-3- | |
| | yl)cyclopropanecarboxamide | |
| Example 959 | 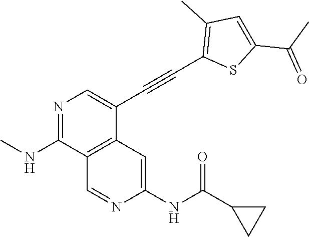 [SMILES: CNc1ncc(C#Cc2sc(C(C)=O)cc2C)c2cc(NC(=O)C3CC3)ncc12] | LCMS (ESI) m/z 405.1 [M + H]⁺; ¹H NMR (400 MHz, DMSO-d₆) δ ppm 11.11 (s, 1H), 9.40 (s, 1H), 8.59 (s, 1H), 8.50 (br s, 1H), 8.34 (s, 1H), 7.86 (s, 1H), 3.03 (d, J = 4.4 Hz, 3H), 2.54 (s, 3H), 2.42 (s, 3H), 2.03-2.12 (m, 1H), 2.03-2.12 (m, 1H), 0.81- 0.91 (m, 4H). |
| | N-(5-((5-acetyl-3-methylthiophen-2- | |
| | yl)ethynyl)-8-(methylamino)-2,7- | |
| | naphthyridin-3- | |
| | yl)cyclopropanecarboxamide. | |
| Example 960 | 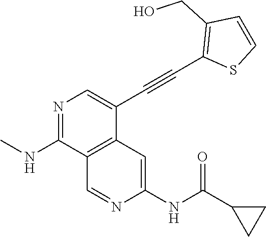 [SMILES: CNc1ncc(C#Cc2sccc2CO)c2cc(NC(=O)C3CC3)ncc12] | LCMS (ESI) m/z 379.2 [M + H]⁺; ¹H NMR (500 MHz, CD₃OD) δ ppm 9.18 (s, 1H), 8.63 (s, 1H), 8.14 (s, 1H), 7.39 (d, J = 5.19 Hz, 1H), 7.16 (d, J = 5.19 Hz, 1H), 4.81 (s, 2H), 3.09 (s, 3H), 1.90-1.99 (m, 1H), 1.00-1.07 (m, 2H), 0.88- 0.96 (m, 2H). |
| | N-(5-((3-(hydroxymethyl)thiophen-2- | |
| | yl)ethynyl)-8-(methylamino)-2,7- | |
| | naphthyridin-3- | |
| | yl)cyclopropanecarboxamide | |
| Example 961 | 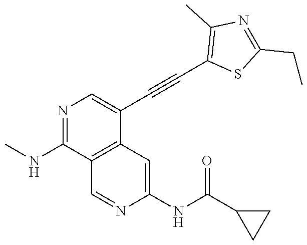 [SMILES: CCc1nc(C)c(C#Cc2cnc(NC)c3cnc(NC(=O)C4CC4)cc23)s1] | LCMS (ESI) m/z 392.2 [M + H]⁺; ¹H NMR (500 MHz, CD₃OD) δ ppm 9.20 (s, 1H), 8.63 (s, 1H), 8.17 (s, 1H), 3.10 (s, 3H), 3.00 (q, J = 7.63 Hz, 2H), 2.55 (s, 3H), 1.91-1.99 (m, 1H), 1.38 (t, J = 7.55 Hz, 3H), 0.98-1.08 (m, 2H), 0.92 (m, 2H). |
| | N-(5-((2-ethyl-4-methylthiazol-5- | |
| | yl)ethynyl)-8-(methylamino)-2,7- | |
| | naphthyridin-3- | |
| | yl)cyclopropanecarboxamide | |
| Example 962 | 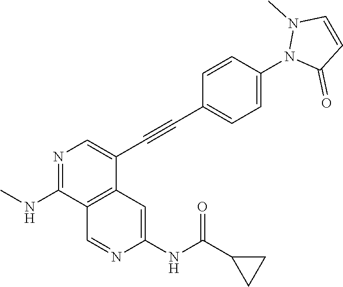 [SMILES: CNc1ncc(C#Cc2ccc(-n3c(=O)ccn3C)cc2)c2cc(NC(=O)C3CC3)ncc12] | LCMS (ESI) m/z 439.2 [M + H]⁺; ¹H NMR (400 MHz, DMSO-d₆) δ ppm 11.09 (s, 1H), 9.37 (s, 1H), 8.67 (s, 1H), 8.35 (br d, J = 4.4 Hz, 1H), 8.29 (s, 1H), 7.98 (d, J = 3.5 Hz, 1H), 7.66 (d, J = 8.5 Hz, 2H), 7.42 (d, J = 8.5 Hz, 2H), 5.49 (d, J = 3.5 Hz, 1H), 3.33 (s, 3H), 3.02 (d, J = 4.1 Hz, 3H), 2.08 (br d, J = 4.4 Hz, 1H), 0.90 (br d, J = 3.8 Hz, 2H), 0.87-0.80 (m, 2H). |
| | N-[8-(methylamino)-5-[2-[4-(2-methyl- | |
| | 5-oxo-pyrazol-1-yl)phenyl]ethynyl]-2,7- | |
| | naphthyridin-3- | |
| | yl]cyclopropanecarboxamide | |
| Example 963 | 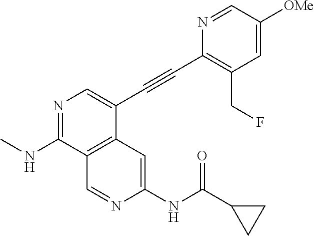 [SMILES: CNc1ncc(C#Cc2ncc(OC)cc2CF)c2cc(NC(=O)C3CC3)ncc12] | LCMS (ESI) m/z 406.1 [M + H]⁺; ¹H NMR (400 MHz, DMSO-d₆) δ ppm 11.11 (s, 1H), 9.38 (s, 1H), 8.59 (s, 1H), 8.42 (br d, J = 4.52 Hz, 1H), 8.35-8.30 (m, 2H), 7.53 (d, J = 2.76 Hz, 1H), 5.81 (s, 1H), 5.70 (s, 1H), 3.91 (s, 3H), 3.02 (d, J = 4.27 Hz, 3H), 2.16- 2.02 (m, 1H), 0.91-0.79 (m, 4H). |
| | N-[5-[2-[3-(fluoromethyl)-5-methoxy-2- | |
| | pyridyl]ethynyl]-8-(methylamino)-2,7- | |
| | naphthyridin-3- | |
| | yl]cyclopropanecarboxamide | |
| Example 964 | 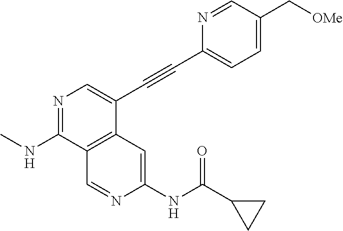 [SMILES: CNc1ncc(C#Cc2ccc(COC)cn2)c2cc(NC(=O)C3CC3)ncc12] | LCMS (ESI) m/z 388.1 [M + H]⁺; ¹H NMR (400 MHz, CD₃OD) δ ppm 9.21 (s, 1H), 8.71 (s, 1H), 8.50 (s, 1H), 8.30 (s, 1H), 7.90- 7.82 (m, 1H), 7.80-7.73 (m, 1H), 4.54 (s, 2H), 3.44 (s, 3H), 3.11 (s, 3H), 2.03-1.91 (m, 1H), 1.07-1.03 (m, 2H), 0.96-0.91 (m, 2H). |
| | N-[5-[2-[5-(methoxymethyl)-2- | |
| | pyridyl]ethynyl]-8-(methylamino)-2,7- | |
| | naphthyridin-3- | |
| | yl]cyclopropanecarboxamide | |
| Example 965 | 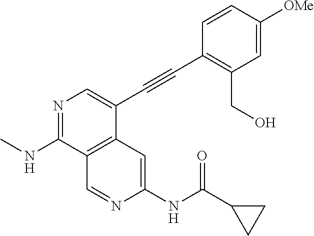 [SMILES: CNc1ncc(C#Cc2ccc(OC)cc2CO)c2cc(NC(=O)C3CC3)ncc12] | LCMS (ESI) m/z 403.1 [M + H ]⁺; ¹H NMR (400 MHz, DMSO-d₆) δ ppm 11.07 (s, 1H), 9.36 (s, 1H), 8.62 (s, 1H), 8.31-8.26 (m, 1H), 8.24 (s, 1H), 7.44 (d, J = 8.50 Hz, 1H), 7.13 (d, J = 2.50 Hz, 1H), 6.87 (dd, J = 8.38, 2.63 Hz, 1H), 5.30 (br t, J = 5.38 Hz, 1H), 4.75 (d, J = 4.50 Hz, 2H), 3.81 (s, 3H), 3.01 (d, J = 4.38 Hz, 3H), 2.12-2.01 (m, 1H), 0.90-0.80 (m, 4H). |
| | N-[5-[2-[2-(hydroxymethyl)-4-methoxy- | |
| | phenyl]ethynyl]-8-(methylamino)-2,7- | |
| | naphthyridin-3- | |
| | yl]cyclopropanecarboxamide | |
| Example 966 | 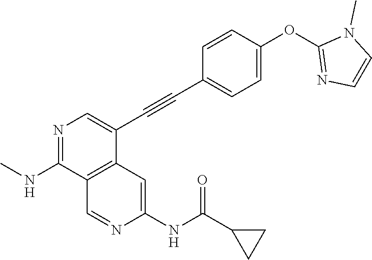 [SMILES: CNc1ncc(C#Cc2ccc(Oc3nccn3C)cc2)c2cc(NC(=O)C3CC3)ncc12] | LCMS (ESI) m/z 439.1 [M + H]⁺; ¹H NMR (400 MHz, DMSO-d₆) δ ppm 11.08 (s, 1H), 9.37 (s, 1H), 8.64 (s, 1H), 8.33 (br d, J = 4.8 Hz, 1H), 8.27 (s, 1H), 7.58 (br d, J = 8.8 Hz, 2H), 7.25 (br d, J = 8.5 Hz, 2H), 6.99 (s, 1H), 6.65 (s, 1H), 3.50 (s, 3H), 3.01 (d, J = 4.3 Hz, 3H), 2.09-2.04 (m, 1H), 0.88 (br s, 2H), 0.85-0.79 (m, 2H). |
| | N-(5-((4-((1-methyl-1H-imidazol-2- | |
| | yl)oxy)phenyl)ethynyl)-8- | |
| | (methylamino)-2,7-naphthyridin-3- | |
| | yl)cyclopropanecarboxamide | |
| Example 967 | 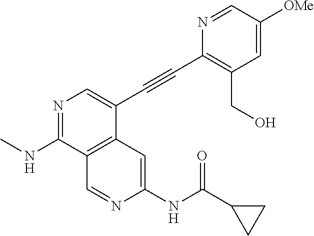 [SMILES: CNc1ncc(C#Cc2ncc(OC)cc2CO)c2cc(NC(=O)C3CC3)ncc12] | LCMS (ESI) m/z 404.1 [M + H]⁺; ¹H NMR (400 MHz, DMSO-d₆) δ ppm 11.09 (s, 1H), 9.38 (s, 1H), 8.59 (s, 1H), 8.38 (br d, J = 4.5 Hz, 1H), 8.31 (s, 1H), 8.19 (d, J = 2.8 Hz, 1H), 7.48 (d, J = 2.8 Hz, 1H), 5.41 (br s, 1H), 4.79 (s, 2H), 3.89 (s, 3H), 3.02 (d, J = 4.3 Hz, 3H), 2.02-2.12 (m, 1H), 0.91-0.79 (m, 4H). |
| | N-[5-[2-[3-(hydroxymethyl)-5-methoxy- | |
| | 2-pyridyl]ethynyl]-8-(methylamino)- | |
| | 2,7-naphthyridin-3- | |
| | yl]cyclopropanecarboxamide | |
| Example 968 | 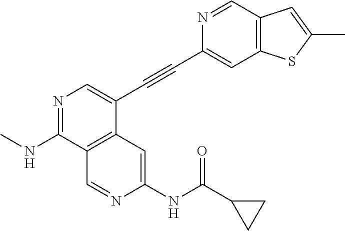 | LCMS (ESI) m/z 414.1 [M + H]⁺; ¹H NMR (400 MHz, DMSO-d₆) δ ppm 11.11 (s, 1H), 9.39 (s, 1H), 8.98 (s, 1H), 8.66 (s, 1H), 8.43- 8.38 (m, 1H), 8.34 (s, 1H), 8.25 (s, 1H), 7.33 (s, 1H), 3.03 (d, J = 4.38 Hz, 3H), 2.63 (d, J = 0.75 Hz, 3H), 2.12-2.05 (m, 1H), 0.89-0.83 (m, 4H). |
| | N-[8-(methylamino)-5-[2-(2- | |
| | methylthieno[3,2-c]pyridin-6- | |
| | yl)ethynyl]-2,7-naphthyridin-3- | |
| | yl]cyclopropanecarboxamide | |
| Example 969 | 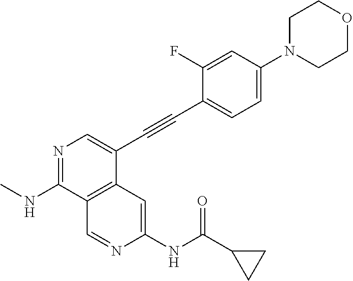 [SMILES: CNc1ncc(C#Cc2ccc(N3CCOCC3)cc2F)c2cc(NC(=O)C3CC3)ncc12] | LCMS (ESI) m/z 446.0 [M + H]⁺; ¹H NMR (400 MHz, DMSO-d₆) δ ppm 11.05 (s, 1H), 9.36 (s, 1H), 8.59 (s, 1H), 8.31 (br d, J = 4.0 Hz, 1H), 8.23 (s, 1H), 7.42 (t, J = 8.8 Hz, 1H), 6.93-6.78 (m, 2H), 3.78-3.67 (m, 4H), 3.27-3.20 (m, 4H), 3.04-2.98 (m, 3H), 2.06 (br d, J = 4.4 Hz, 1H), 0.89- 0.80 (m, 4H). |
| | N-[5-[2-(2-fluoro-4-morpholino- | |
| | phenyl)ethynyl]-8-(methylamino)-2,7- | |
| | naphthyridin-3- | |
| | yl]cyclopropanecarboxamide | |
| Example 970 | 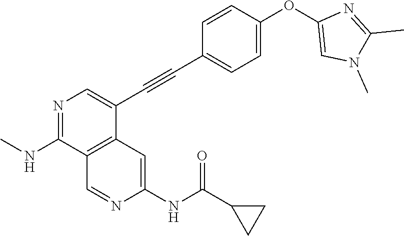 | LCMS (ESI) m/z 453.1 [M + H]⁺; ¹H NMR (400 MHz, DMSO-d₆) δ ppm 11.07 (s, 1H), 9.36 (s, 1H), 8.63 (s, 1H), 8.33-8.26 (m, 1H), 8.25 (s, 1H), 7.49 (d, J = 8.53 Hz, 2H), 7.02 (d, J = 8.78 Hz, 2H), 6.78 (s, 1H), 3.54 (s, 3H), 3.04- 2.99 (m, 3H), 2.25 (s, 3H), 2.13- 2.01 (m, 1H), 0.90-0.81 (m, 4H). |
| | N-(5-((4-((1,2-dimethyl-1H-imidazol-4- | |
| | yl)oxy)phenyl)ethynyl)-8- | |
| | (methylamino)-2,7-naphthyridin-3- | |
| | yl)cyclopropanecarboxamide | |
| Example 971 | 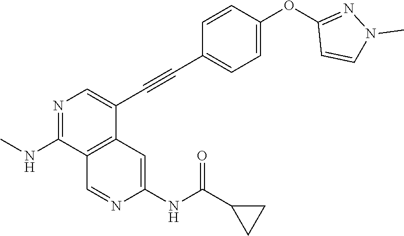 | LCMS (ESI) m/z 439.1 [M + H]⁺; ¹H NMR (400 MHz, DMSO-d₆) δ ppm 10.77 (br s, 1H), 9.34 (s, 1H), 8.62 (s, 1H), 8.25 (s, 1H), 8.10 (br d, J = 2.3 Hz, 1H), 7.62 (d, J = 2.3 Hz, 1H), 7.54 (d, J = 8.8 Hz, 2H), 7.12 (d, J = 8.8 Hz, 2H), 5.88 (d, J = 2.3 Hz, 1H), 3.77 (s, 3H), 3.04 (d, J = 4.5 Hz, 3H), 2.14-2.04 (m, 1H), 0.94- 0.89 (m, 2H), 0.84 (dt, J = 7.8, 3.0 Hz, 2H). |
| | N-[8-(methylamino)-5-[2-[4-(1- | |
| | methylpyrazol-3-yl)oxyphenyl]ethynyl]- | |
| | 2,7-naphthyridin-3- | |
| | yl]cyclopropanecarboxamide | |
| Example 972 | 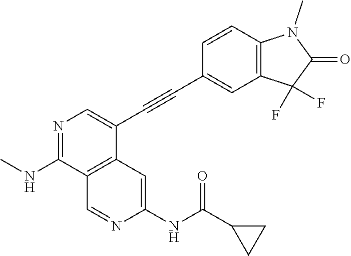 [SMILES: CNc1ncc(C#Cc2ccc3c(c2)C(F)(F)C(=O)N3C)c2cc(NC(=O)C3CC3)ncc12] | LCMS (ESI) m/z 448.0 [M + H]⁺; ¹H NMR (400 MHz, DMSO-d₆) δ ppm 11.13 (s, 1H), 9.37 (s, 1H), 8.70 (s, 1H), 8.40-8.34 (m, 1H), 8.27 (s, 1H), 7.95 (s, 1H), 7.79 (d, J = 8.23 Hz, 1H), 7.32 (d, J = 8.23 Hz, 1H), 3.21 (s, 3H), 3.01 (d, J = 4.29 Hz, 3H), 2.12-2.04 (m, 1H), 0.92-0.82 (m, 4H). |
| | N-[5-[2-(3,3-difluoro-1-methyl-2-oxo- | |
| | indolin-5-yl)ethynyl]-8-(methylamino)- | |
| | 2,7-naphthyridin-3- | |
| | yl]cyclopropanecarboxamide | |
| Example 973 | 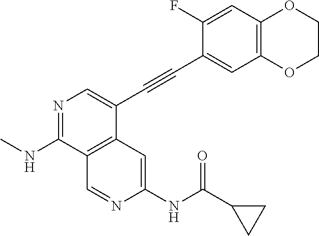 [SMILES: CNc1ncc(C#Cc2cc3c(cc2F)OCCO3)c2cc(NC(=O)C3CC3)ncc12] | LCMS (ESI) m/z 419.0 [M + H]⁺; ¹H NMR (400 MHz, DMSO-d₆) δ ppm 11.05 (s, 1H), 9.36 (s, 1H), 8.57 (s, 1H), 8.33 (br d, J = 4.5 Hz, 1H), 8.24 (s, 1H), 7.07 (d, J = 7.0 Hz, 1H), 6.93 (d, J = 10.0 Hz, 1H), 4.33-4.28 (m, 2H), 4.28-4.22 (m, 2H), 3.01 (d, J = 4.3 Hz, 3H), 2.10-2.03 (m, 1H), 0.89-0.80 (m, 4H). |
| | N-[5-[2-(6-fluoro-2,3-dihydro-1,4- | |
| | benzodioxin-7-yl)ethynyl]-8- | |
| | (methylamino)-2,7-naphthyridin-3- | |
| | yl]cyclopropanecarboxamide | |
| Example 974 | 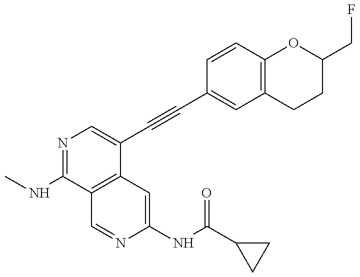 [SMILES: CNc1ncc(C#Cc2ccc3c(c2)CCC(CF)O3)c2cc(NC(=O)C3CC3)ncc12] | LCMS (ESI) m/z 431.1 [M + H]⁺; ¹H NMR (400 MHz, DMSO-d₆) δ ppm 11.07 (s, 1H), 9.36 (s, 1H), 8.64 (s, 1H), 8.27 (br d, J = 4.8 Hz, 1H), 8.22 (s, 1H), 7.35 (s, 1H), 7.26 (dd, J = 8.4, 1.9 Hz, 1H), 6.84 (d, J = 8.5 Hz, 1H), 4.77-4.50 (m, 2H), 4.42-4.28 (m, 1H), 3.05-2.95 (m, 3H), 2.93-2.73 (m, 2H), 2.15-1.97 (m, 2H), 1.85-1.67 (m, 1H), 0.92-0.81 (m, 4H). |
| | N-[5-[2-[2-(fluoromethyl)chroman-6- | |
| | yl]ethynyl]-8-(methylamino)-2,7- | |
| | naphthyridin-3- | |
| | yl]cyclopropanecarboxamide | |
| Example 975 | 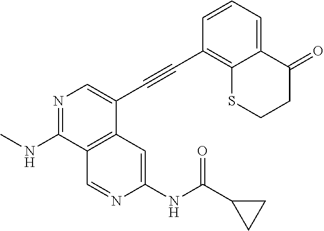 [SMILES: CNc1ncc(C#Cc2cccc3c2SCCC3=O)c2cc(NC(=O)C3CC3)ncc12] | LCMS (ESI) m/z 429.0 [M + H]⁺; ¹H NMR (400 MHz, DMSO-d₆) δ ppm 11.08 (s, 1H), 9.39 (s, 1H), 8.67 (s, 1H), 8.43 (br d, J = 4.8 Hz, 1H), 8.31 (s, 1H), 7.96 (d, J = 7.3 Hz, 1H), 7.70 (d, J = 7.5 Hz, 1H), 7.27 (t, J = 7.7 Hz, 1H), 3.36 (br s, 2H), 3.03 (d, J = 4.3 Hz, 3H), 2.97-2.91 (m, 2H), 2.07 (br t, J = 4.9 Hz, 1H), 0.81- 0.89 (m, 4H). |
| | N-[8-(methylamino)-5-[2-(4- | |
| | oxothiochroman-8-yl)ethynyl]-2,7- | |
| | naphthyridin-3- | |
| | yl]cyclopropanecarboxamide | |
| Example 976 | 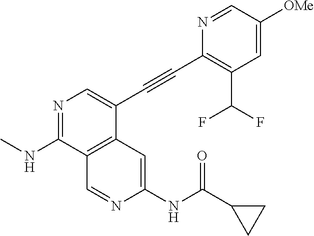 [SMILES: CNc1ncc(C#Cc2ncc(OC)cc2C(F)F)c2cc(NC(=O)C3CC3)ncc12] | LCMS (ESI) m/z 424.0 [M + H]⁺; ¹H NMR (400 MHz, DMSO-d₆) δ ppm 11.12 (s, 1H), 9.38 (s, 1H), 8.57 (s, 1H), 8.51-8.43 (m, 2H), 8.38 (s, 1H), 7.62 (d, J = 2.8 Hz, 1H), 7.56-7.28 (m, 1H), 3.94 (s, 3H), 3.03 (d, J = 4.4 Hz, 3H), 2.10-2.04 (m, 1H), 0.88-0.83 (m, 4H). |
| | N-(5-((3-(difluoromethyl)-5- | |
| | methoxypyridin-2-yl)ethynyl)-8- | |
| | (methylamino)-2,7-naphthyridin-3- | |
| | yl)cyclopropanecarboxamide | |
| Example 977 | 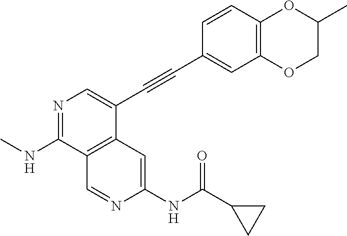 | LCMS (ESI) m/z 414.9 [M + H]⁺; ¹H NMR (400 MHz, DMSO-d₆) δ ppm 11.06 (s, 1 H), 9.35 (s, 1 H), 8.61 (s, 1 H), 8.28 (br d, J = 4.4 Hz, 1 H), 8.23 (s, 1H), 7.06- 7.01 (m, 2 H), 6.90 (d, J = 8.8 Hz, 1 H), 4.37-4.29 (m, 2 H), 3.90-3.82 (m, 1 H), 3.00 (d, J = 4.4 Hz, 3 H), 2.11-2.04 (m, 1 H), 1.30 (d, J = 6.4 Hz, 3H), 0.91-0.86 (m, 2 H), 0.85-0.80 (m, 2 H). |
| | N-[8-(methylamino)-5-[2-(2-methyl-2,3- | |
| | dihydro-1,4-benzodioxin-6-yl)ethynyl]- | |
| | 2,7-naphthyridin-3- | |
| | yl]cyclopropanecarboxamide | |
| Example 978 | 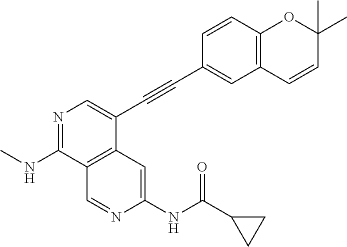 | LCMS (ESI) m/z 425.0 [M + H]⁺; ¹H NMR (400 MHz, DMSO-d₆) δ 11.07 (s, 1H), 9.35 (s, 1H), 8.63 (s, 1H), 8.28 (br d, J = 4.8 Hz, 1H), 8.22 (s, 1H), 7.32 (d, J = 2.0 Hz, 1H), 7.28 (dd, J = 2.0, 8.3 Hz, 1H), 6.79 (d, J = 8.4 Hz, 1H), 6.42 (d, J = 9.6 Hz, 1H), 5.83 (d, J = 9.6 Hz, 1H), 3.00 (d, J = 4.0 Hz, 3H), 2.07 (s, 1H), 1.40 (s, 6 H), 0.90-0.87 (m, 2H), 0.85- 0.82 (m, 2H). |
| | N-[5-[2-(2,2-dimethylchromen-6- | |
| | yl)ethynyl]-8-(methylamino)-2,7- | |
| | naphthyridin-3- | |
| | yl]cyclopropanecarboxamide | |
| Example 979 | 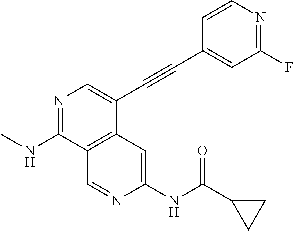 [SMILES: CNc1ncc(C#Cc2ccnc(F)c2)c2cc(NC(=O)C3CC3)ncc12] | LCMS (ESI) m/z 362.0 [M + H]⁺; ¹H NMR (400 MHz, DMSO-d₆) δ ppm 11.14 (s, 1H), 9.38 (s, 1H), 8.66 (s, 1H), 8.54 (br d, J = 4.5 Hz, 1H), 8.35 (s, 1H), 8.30 (d, J = 5.1 Hz, 1H), 7.44 (dt, J = 5.0, 1.6 Hz, 1H), 7.29 (s, 1H), 3.03 (d, J = 4.1 Hz, 3H), 2.13-2.05 (m, 1H), 0.92-0.84 (m, 4H). |
| | N-[5-[2-(2-fluoro-4-pyridyl)ethynyl]-8- | |
| | (methylamino)-2,7-naphthyridin-3- | |
| | yl]cyclopropanecarboxamide | |
| Example 980 | 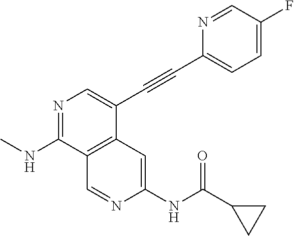 [SMILES: CNc1ncc(C#Cc2ccc(F)cn2)c2cc(NC(=O)C3CC3)ncc12] | LCMS (ESI) m/z 362.2 [M + H]⁺; ¹H NMR (400 MHz, DMSO-d₆) δ ppm 11.07 (s, 1H), 9.38 (s, 1H), 8.67-8.57 (m, 2H), 8.41 (br d, J = 4.3 Hz, 1H), 8.35 (s, 1H), 7.87- 7.79 (m, 1H), 7.70 (dd, J = 8.8, 4.5 Hz, 1H), 3.06-3.00 (m, 3H), 2.12-2.04 (m, 1H), 0.91-0.81 (m, 4H). |
| | N-[5-[2-(5-fluoro-2-pyridyl)ethynyl]-8- | |
| | (methylamino)-2,7-naphthyridin-3- | |
| | yl]cyclopropanecarboxamide | |
| Example 981 | 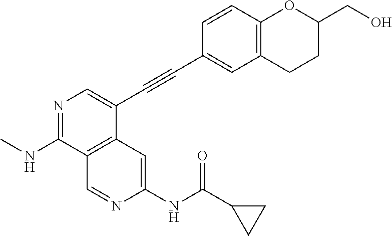 [SMILES: CNc1ncc(C#Cc2ccc3c(c2)CCC(CO)O3)c2cc(NC(=O)C3CC3)ncc12] | LCMS (ESI) m/z 429.3 [M + H]⁺; ¹H NMR (400 MHz, CD₃OD) δ ppm 9.18 (s, 1H), 8.71 (s, 1H), 8.13 (s, 1H), 7.35 (s, 1H), 7.31 (d, J = 8.4 Hz, 1H), 6.80 (d, J = 8.4 Hz, 1H), 4.05-4.13 (m, 1H), 3.75 (d, J = 5.2 Hz, 2H), 3.08 (s, 3H), 2.80-2.98 (m, 2H), 2.03- 2.10 (m, 1H), 1.93-2.00 (m, 1H), 1.73-1.88 (m, 1H), 1.03-1.10 (m, 2H), 0.92-0.96 (m, 2H). |
| | N-[5-[2-[2-(hydroxymethyl)chroman-6- | |
| | yl]ethynyl]-8-(methylamino)-2,7- | |
| | naphthyridin-3- | |
| | yl]cyclopropanecarboxamide | |
| Example 982 | 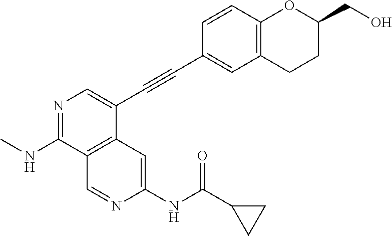 [SMILES: CNc1ncc(C#Cc2ccc3c(c2)CC[C@H](CO)O3)c2cc(NC(=O)C3CC3)ncc12] | LCMS (ESI) m/z 429.3 [M + H]⁺; ¹H NMR (400 MHz, CD₃OD) δ ppm, 9.18 (s, 1H), 8.70 (s, 1H), 8.13 (s, 1H), 7.27-7.39 (m, 2H), 6.80 (br d, J = 8.4 Hz, 1H), 4.10 (br s, 1H), 3.74 (br d, J = 4.78 Hz, 2H), 3.08 (s, 3H), 2.78-2.98 (m, 2H), 2.04 (br s, 1H), 1.96 (br s, 1H), 1.84 (br d, J = 10.4 Hz, 1H), 1.06 (br d, J = 2.8 Hz, 2H), 0.89-0.98 (m, 2H). |
| | (S)-N-(5-(6-(2- | |
| | (methoxymethyl)morpholino)- | |
| | [1,2,4]triazolo[1,5-a]pyridin-2-yl)-8- | |
| | (methylamino)-2,7-naphthyridin-3- | |
| | yl)cyclopropanecarboxamide | |
| | Isomer obtained by chiral SFC | |
| | purification of example 981; the | |
| | configuration has been arbitrarily | |
| | assigned. | |
| Example 983 | 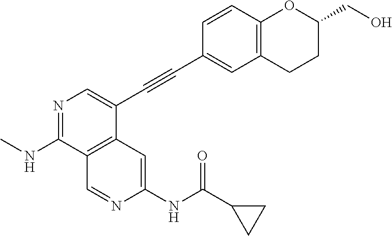 [SMILES: CNc1ncc(C#Cc2ccc3c(c2)CC[C@@H](CO)O3)c2cc(NC(=O)C3CC3)ncc12] | LCMS (ESI) m/z 429.3 [M + H]⁺; ¹H NMR (400 MHz, CD₃OD) δ ppm, 9.18 (s, 1H), 8.70 (s, 1H), 8.13 (s, 1H), 7.35 (s, 1H), 7.31 (br d, J = 8.4 Hz, 1H), 6.80 (d, J = 8.4 Hz, 1H), 4.02-4.18 (m, 1H), 3.75 (d, J = 5.2 Hz, 2H), 3.08 (s, 3H), 2.79-2.95 (m, 2H), 2.05 (br s, 1H), 1.92-1.99 (m, 1H), 1.75-1.87 (m, 1H), 1.03- 1.08 (m, 2H), 0.93 (d, J = 7.8, 2H). |
| | (R)-N-(5-(6-(2- | |
| | (methoxymethyl)morpholino)- | |
| | [1,2,4]triazolo[1,5-a]pyridin-2-yl)-8- | |
| | (methylamino)-2,7-naphthyridin-3- | |
| | yl)cyclopropanecarboxamide | |
| | Isomer obtained by chiral SFC | |
| | purification of example 981; the | |
| | configuration has been arbitrarily | |
| | assigned. | |
| Example 984 | 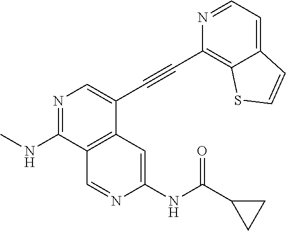 [SMILES: CNc1ncc(C#Cc2nccc3ccsc23)c2cc(NC(=O)C3CC3)ncc12] | LCMS (ESI) m/z 400.2 [M + H]⁺; ¹H NMR (400 MHz, DMSO-d₆) δ ppm 11.09 (s, 1H), 9.41 (s, 1H), 8.66 (s, 1H), 8.52 (br s, 2H), 8.44 (s, 1H), 8.19 (d, J = 5.2 Hz, 1H), 7.88 (d, J = 5.6 Hz, 1H), 7.63 (d, J = 5.2 Hz, 1H), 3.04-3.07 (m, 3H), 2.08 (br s, 1H), 0.82-0.88 (m, 4H). |
| | N-[8-(methylamino)-5-(2-thieno[2,3- | |
| | c]pyridin-7-ylethynyl)-2,7- | |
| | naphthyridin-3- | |
| | yl]cyclopropanecarboxamide | |
| Example 985 | 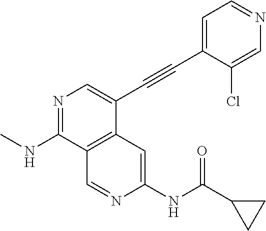 [SMILES: CNc1ncc(C#Cc2ccncc2Cl)c2cc(NC(=O)C3CC3)ncc12] | LCMS (ESI) m/z 378.0 [M + H]⁺; ¹H NMR (400 MHz, DMSO-d₆) δ ppm 11.09 (s, 1H), 9.39 (s, 1H), 8.79 (br s, 1H), 8.66 (s, 1H), 8.57 (br d, J = 4.8 Hz, 2H), 8.39 (s, 1H), 7.61 (br s, 1H), 3.04 (d, J = 4.4 Hz, 3H), 2.08 (s, 1H), 0.82- 0.89 (m, 4H). |
| | N-[5-[2-(3-chloro-4-pyridyl)ethynyl]-8- | |
| | (methylamino)-2,7-naphthyridin-3- | |
| | yl]cyclopropanecarboxamide | |
| Example 986 | 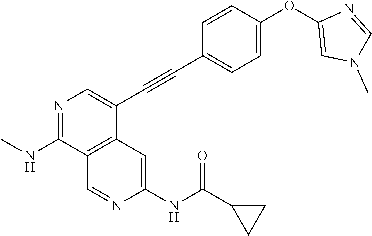 [SMILES: CNc1ncc(C#Cc2ccc(Oc3cn(C)cn3)cc2)c2cc(NC(=O)C3CC3)ncc12] | LCMS (ESI) m/z 439.1 [M + H]⁺; ¹H NMR (400 MHz, DMSO-d₆) δ ppm, 11.08 (s, 1H), 9.36 (s, 1H), 8.63 (s, 1H), 8.31 (br d, J = 4.0 Hz, 1H), 8.25 (s, 1H), 7.50 (d, J = 8.8 Hz, 2H), 7.45 (s, 1H), 7.03 (d, J = 8.8 Hz, 2H), 6.92 (d, J = 1.6 Hz, 1H), 3.65 (s, 3H), 3.01 (d, J = 4.0 Hz, 3H), 2.07 (br d, J = 3.6 Hz, 1H), 0.88 (br s, 2H), 0.82-0.86 (m, 2H). |
| | N-[8-(methylamino)-5-[2-[4-(1- | |
| | methylimidazol-4- | |
| | yl)oxyphenyl]ethynyl]-2,7- | |
| | naphthyridin-3- | |
| | yl]cyclopropanecarobxmaide | |
| Example 987 | 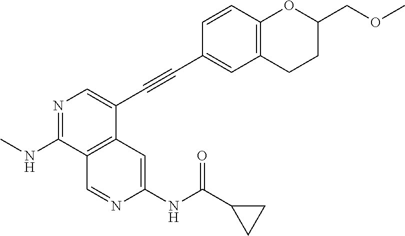 | LCMS (ESI) m/z 443.3 [M + H]⁺, ¹H NMR (400 MHz, CD₃OD) δ ppm 9.18 (s, 1H), 8.71 (s, 1H), 8.13 (s, 1H), 7.35 (s, 1H), 7.28- 7.33 (m, 1H), 6.78 (d, J = 8.4 Hz, 1H), 4.17-4.26 (m, 1H), 3.58- 3.67 (m, 2H), 3.44 (s, 3H), 3.08 (s, 3H), 2.86-2.95 (m, 1H), 2.76- 2.85 (m, 1H), 2.02-2.11 (m, 1H), 1.92-2.01 (m, 1H), 1.74-1.87 (m, 1H), 1.06 (quin, J = 3.6 Hz, 2H), 0.91-0.97 (m, 2H). |
| | N-[5-[2-[2-(methoxymethyl)chroman-6- | |
| | yl]ethynyl]-8-(methylamino)-2,7- | |
| | naphthyridin-3- | |
| | yl]cyclopropanecarobxamide | |
| Example 988 | 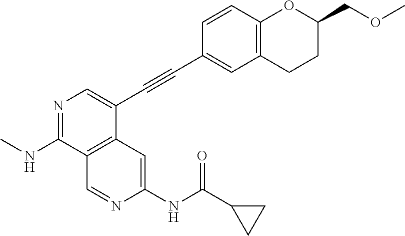 | LCMS (ESI) m/z 443.3 [M + H]⁺; ¹H NMR (400 MHz, CD₃OD) δ ppm 9.18 (s, 1H), 8.70 (s, 1H), 8.12 (s, 1H), 7.35 (s, 1H), 7.30 (br d, J = 8.8 Hz, 1H), 6.78 (d, J = 8.4 Hz, 1H), 4.13-4.28 (m, 1H), 3.55-3.70 (m, 2H), 3.44 (s, 3H), 3.08 (s, 3H), 2.77-2.97 (m, 2H), 2.00-2.11 (m, 1H), 1.91-1.99 (m, 1H), 1.73-1.88 (m, 1H), 1.01- 1.11 (m, 2H), 0.87-0.97 (m, 2H). |
| | N-[5-[2-[(2R)-2- | |
| | (methoxymethyl)chroman-6- | |
| | yl]ethynyl]-8-(methylamino)-2,7- | |
| | naphthyridin-3- | |
| | yl]cyclopropanecarboxamide | |
| | Isomer obtained by chiral SFC | |
| | purification of example 987, the | |
| | configuration has been arbitrarily | |
| | assigned. | |
| Example 989 | 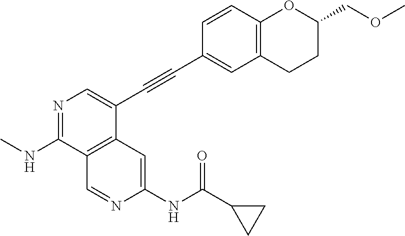 | LCMS (ESI) m/z 443.3 [M + H]⁺; ¹H NMR (400 MHz, CD₃OD) δ ppm 9.18 (s, 1H), 8.70 (s, 1H), 8.12 (s, 1H), 7.35 (s, 1H), 7.31 (br d, J = 8.4 Hz, 1H), 6.78 (d, J = 8.4 Hz, 1H), 4.13-4.27 (m, 1H), 3.55-3.69 (m, 2H), 3.44 (s, 3H), 3.08 (s, 3H), 2.75-2.97 (m, 2H), 2.02-2.12 (m, 1H), 1.90-2.01 (m, 1H), 1.72-1.87 (m, 1H), 1.01- 1.11 (m, 2H), 0.93 (m 2H). |
| | N-[5-[2-[(2S)-2- | |
| | (methoxymethyl)chroman-6- | |
| | yl]ethynyl]-8-(methylamino)-2,7- | |
| | naphthyridin-3- | |
| | yl]cyclopropanecarboxamide | |
| | Isomer obtained by chiral SFC | |
| | purification of example 987; the | |
| | configuration has been arbitrarily | |
| | assigned. | |
| Example 990 | 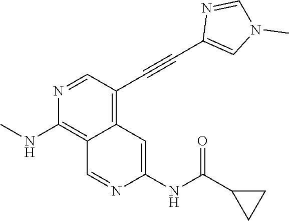 | LCMS (ESI) m/z 347.1 [M + H]⁺; ¹H NMR (400 MHz, DMSO-d₆) δ ppm 11.05 (s, 1H), 9.36 (s, 1H), 8.53 (s, 1H), 8.30 (br d, J = 4.0 Hz, 1H), 8.22 (s, 1H), 7.68 (br s, 1H), 7.51 (br s, 1H), 3.68 (s, 3H), 3.00 (br d, J = 3.6 Hz, 3H), 2.06 (br s, 1H), 0.78-0.90 (m, 4H). |
| | N-[8-(methylamino)-5-[2-(1- | |
| | methylimidazol-4-yl)ethynyl]-2,7- | |
| | naphthyridin-3- | |
| | yl]cyclopropanecarboxamide | |
| Example 991 | 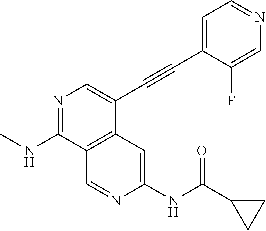 [SMILES: CNc1ncc(C#Cc2ccncc2F)c2cc(NC(=O)C3CC3)ncc12] | LCMS (ESI) m/z 362.2 [M + H]⁺; ¹H NMR (400 MHz, DMSO-d₆) δ ppm 11.11 (s, 1H), 9.39 (s, 1H), 8.70 (s, 1H), 8.61 (s, 1H), 8.54 (br d, J = 4.4 Hz, 1H), 8.49 (d, J = 4.8 Hz, 1H), 8.37 (s, 1H), 7.57- 7.64 (m, 1H), 3.04 (d, J = 4.2 Hz, 3H), 2.01-2.14 (m, 1H), 0.87- 0.92 (m, 2H), 0.81-0.87 (m, 2H). |
| | N-[5-[2-(3-fluoro-4-pyridyl)ethynyl]-8- | |
| | (methylamino)-2,7-naphthyridin-3- | |
| | yl]cyclopropanecarboxamide | |
| Example 992 | 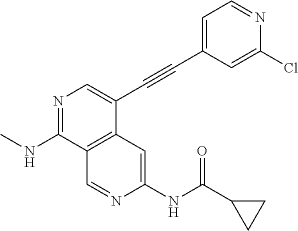 [SMILES: CNc1ncc(C#Cc2ccnc(Cl)c2)c2cc(NC(=O)C3CC3)ncc12] | LCMS (ESI) m/z 378.0 [M + H]⁺; ¹H NMR (400 MHz, DMSO-d₆) δ ppm 11.16 (s, 1H), 9.38 (s, 1H), 8.68 (s, 1H), 8.55 (br d, J = 4.4 Hz, 1H), 8.46 (d, J = 5.2 Hz, 1H), 8.36 (s, 1H), 7.66 (s, 1H), 7.49 (dd, J = 1.2, 5.2 Hz, 1H), 3.03 (d, J = 4.4 Hz, 3H), 2.02-2.14 (m, 1H), 0.83-0.94 (m, 4H). |
| | N-[5-[2-(2-chloro-4-pyridyl)ethynyl]-8- | |
| | (methylamino)-2,7-naphthyridin-3- | |
| | yl]cyclopropanecarboxamide | |
| Example 993 | 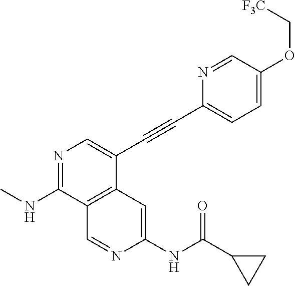 [SMILES: CNc1ncc(C#Cc2ccc(OCC(F)(F)F)cn2)c2cc(NC(=O)C3CC3)ncc12] | LCMS (ESI) m/z 442.0 [M + H]⁺; ¹H NMR (400 MHz, DMSO-d₆) δ ppm, 11.10 (s, 1H), 9.38 (s, 1H), 8.62 (s, 1H), 8.36-8.47 (m, 2H), 8.32 (s, 1H), 7.63 (s, 2H), 4.95 (q, J = 8.8 Hz, 2H), 3.02 (d, J = 4.4 Hz, 3H), 1.23 (s, 1H), 0.83-0.89 (m, 4H). |
| | N-[8-(methylamino)-5-[2-[5-(2,2,2- | |
| | trifluoroethoxy)-2-pyridyl]ethynyl]-2,7- | |
| | naphthyridin-3- | |
| | yl]cyclopropanecarboxamide | |
| Example 995 | 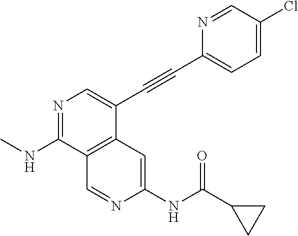 [SMILES: CNc1ncc(C#Cc2ccc(Cl)cn2)c2cc(NC(=O)C3CC3)ncc12] | LCMS (ESI) m/z 378.2 [M + H]⁺; ¹H NMR (400 MHz, DMSO-d₆) δ ppm 11.12 (s, 1H), 9.38 (s, 1H), 8.66 (d, J = 2.4 Hz, 1H), 8.61 (s, 1H), 8.48 (br d, J = 4.4 Hz, 1H), 8.36 (s, 1H), 8.02 (dd, J = 8.4, 2.4 Hz, 1H), 7.64 (d, J = 8.4 Hz, 1H), 3.03 (d, J = 4.0 Hz, 3H), 1.23 (s, 1H), 0.83-0.90 (m, 4H). |
| | N-(5-((5-chloropyridin-2-yl)ethynyl)-8- | |
| | (methylamino)-2,7-naphthyridin-3- | |
| | yl)cyclopropanecarboxamide | |
| Example 996 | 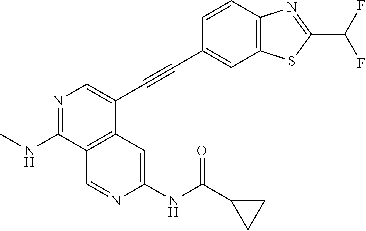 [SMILES: CNc1ncc(C#Cc2ccc3nc(C(F)F)sc3c2)c2cc(NC(=O)C3CC3)ncc12] | LCMS (ESI) m/z 449.9 [M + H]⁺; ¹H NMR (400 MHz, DMSO-d₆) δ ppm 10.89-11.29 (m, 1H), 9.32- 9.47 (m, 1H), 8.64-8.75 (m, 1H), 8.50 (s, 1H), 8.40 (d, J = 4.4 Hz, 1H), 8.29-8.36 (m, 1H), 8.22 (d, J = 8.4 Hz, 1H), 7.79 (d, J = 8.4 Hz, 1H), 7.33-7.70 (m, 1H), 3.03 (d, J = 4.4 Hz, 3H), 2.05- 2.12 (m, 1H), 0.83-0.92 (m, 4H). |
| | N-(5-((2- | |
| | (difluoromethyl)benzo[d]thiazol-6- | |
| | yl)ethynyl)-8-(methylamino)-2,7- | |
| | naphthyridin-3- | |
| | yl)cyclopropanecarboxamide | |
| Example 997 | 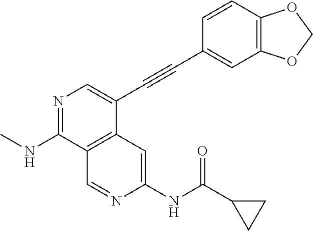 [SMILES: CNc1ncc(C#Cc2ccc3c(c2)OCO3)c2cc(NC(=O)C3CC3)ncc12] | LCMS (ESI) m/z 387.2 [M + H]⁺; ¹H NMR (400 MHz, DMSO-d₆) δ ppm 11.48 (s, 1H), 10.70 (br s, 1H), 9.81 (s, 1H), 8.78 (s, 1H), 8.00 (s, 1H), 7.15 (s, 1H), 7.14 (d, J = 5.6 Hz, 1H), 7.03 (d, J = 8.4 Hz, 1H), 6.11 (s, 2H), 3.19- 3.14 (m, 3H), 2.11 (br s, 1H), 0.95-0.87 (m, 4H). |
| | N-[5-[2-(1,3-benzodioxol-5-yl)ethynyl]- | |
| | 8-(methylamino)-2,7-naphthyridin-3- | |
| | yl]cyclopropanecarboxamide | |
| Example 998 | 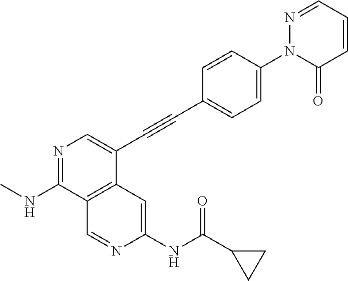 [SMILES: CNc1ncc(C#Cc2ccc(-n3ncccc3=O)cc2)c2cc(NC(=O)C3CC3)ncc12] | LCMS (ESI) m/z 437.1 [M + H]+; 1H NMR (400 MHz, DMSO-d6) δ ppm 11.50 (s, 1H), 10.69 (br d, J = 2.8 Hz, 1H), 9.80 (br s, 1H), 8.83 (s, 1H), 8.07-8.13 (m, 2H), 7.69-7.78 (m, 4H), 7.52 (m, 1H), 7.11 (m, 1H), 3.18 (br d, J = 3.3 Hz, 3H), 2.07-2.18 (m, 1H), 0.87-0.97 (m, 4H). |
| | N-(8-(methylamino)-5-((4-(6- | |
| | oxopyridazin-1(6H)-yl)phenyl)ethynyl)- | |
| | 2,7-naphthyridin-3- | |
| | yl)cyclopropanecarboxamide | |
| Example 999 | 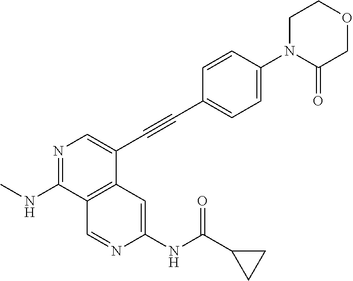 [SMILES: CNc1ncc(C#Cc2ccc(N3CCOCC3=O)cc2)c2cc(NC(=O)C3CC3)ncc12] | LCMS (ESI) m/z 442.4 [M + H]+; 1H NMR (400 MHz, DMSO-d6) δ ppm 11.07 (s, 1H), 9.37 (s, 1H), 8.67 (s, 1H), 8.33 (br d, J = 4.8 Hz, 1H), 8.28 (s, 1H), 7.54-7.62 (m, 2H), 7.47-7.53 (m, 2H), 4.23 (s, 2H), 3.99 (m, 2H), 3.72- 3.85 (m, 2H), 3.02 (d, J = 4.5 Hz, 3H), 2.01-2.16 (m, 1H), 0.76- 0.97 (m, 4H). |
| | N-(8-(methylamino)-5-((4-(3- | |
| | oxomorpholino)phenyl)ethynyl)-2,7- | |
| | naphthyridin-3- | |
| | yl)cyclopropanecarboxamide | |
| Example 1000 | 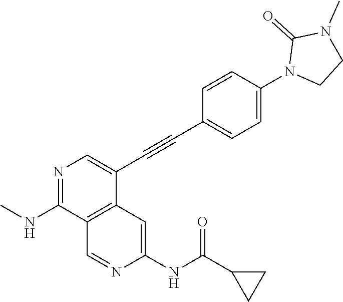 [SMILES: CNc1ncc(C#Cc2ccc(N3CCN(C)C3=O)cc2)c2cc(NC(=O)C3CC3)ncc12] | LCMS (ESI) m/z 441.1 [M + H]+; 1H NMR (400 MHz, DMSO-d6) δ ppm 11.06 (s, 1H), 9.36 (s, 1H), 8.65 (s, 1H), 8.27 (br d, J = 4.8 Hz, 1H), 8.24 (s, 1H), 7.64 (d, J = 8.8 Hz, 2H), 7.50 (d, J = 8.8 Hz, 2H), 3.78-3.86 (m, 2H), 3.44- 3.49 (m, 2H), 3.01 (d, J = 4.0 Hz, 3H), 2.78 (s, 3H), 2.11-2.04 (m, 1H), 0.87-0.93 (m, 2H), 0.81- 0.86 (m, 2H). |
| | N-(5-((4-(3-methyl-2-oxoimidazolidin-1- | |
| | yl)phenyl)ethynyl)-8-(methylamino)- | |
| | 2,7-naphthyridin-3- | |
| | yl)cyclopropanecarboxamide | |
| Example 1001 | 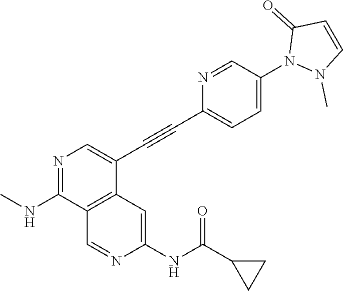 [SMILES: CNc1ncc(C#Cc2ccc(-n3c(=O)ccn3C)cn2)c2cc(NC(=O)C3CC3)ncc12] | LCMS (ESI) m/z 440.1 [M + H]+; 1H NMR (400 MHz, DMSO-d6) δ ppm 11.12 (s, 1H), 9.39 (s, 1H), 8.59-8.67 (m, 2H), 8.45 (br d, J = 4.5 Hz, 1H), 8.37 (s, 1H), 8.06 (d, J = 3.6 Hz, 1H), 7.86 (dd, J = 8.4, 2.5 Hz, 1H), 7.74 (d, J = 8.4 Hz, 1H), 5.54 (d, J = 3.5 Hz, 1H), 3.21 (s, 3H), 3.01-3.05 (m, 3H), 2.03-2.13 (m, 1H), 0.87-0.92 (m, 2H), 0.82-0.87 (m, 2H). |
| | N-(5-((5-(2-methyl-5-oxo-2,5-dihydro- | |
| | 1H-pyrazol-1-yl)pyridine-2-yl)ethynyl)- | |
| | 8-(methylamino)-2,7-nahphthyridin-3- | |
| | yl)cyclopropanecarboxamide | |
| Example 1002 | 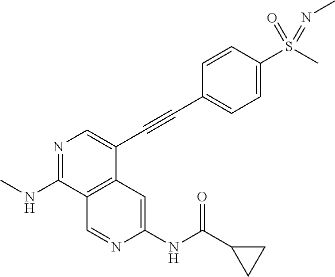 | LCMS (ESI) m/z 433.9 [M + H]+; 1H NMR (400 MHz, DMSO-d6) δ ppm 11.11 (s, 1H), 9.38 (s, 1H), 8.67 (s, 1H), 8.43 (br d, J = 4.8 Hz, 1H), 8.33 (s, 1H), 7.85-7.90 (m, 2H), 7.77 (d, J = 8.4 Hz, 2H), 3.16 (s, 3H), 3.03 (d, J = 4.4 Hz, 3H), 2.48 (s, 3H), 2.06-2.12 (m, 1H), 0.89-0.93 (m, 2H), 0.83- 0.88 (m, 2H). |
| | N-(5-((4-(N,S- | |
| | dimethylsulfonimidoyl)phenyl)ethynyl)- | |
| | 8-(methylamino)-2,7-naphthyridin-3- | |
| | yl)cyclopropanecarobxamide | |
| Example 1003 | 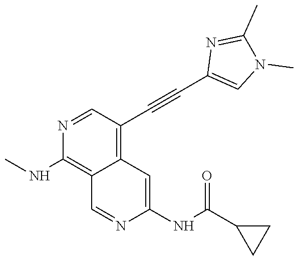 | LCMS (ESI) m/z 361.2 [M + H]+; 1H NMR (400 MHz, DMSO-d6) δ ppm 11.04 (s, 1H), 9.35 (s, 1H), 8.51 (s, 1H), 8.25 (br d, J = 4.77 Hz, 1H), 8.19 (s, 1H), 7.39 (s, 1H), 3.57 (s, 3H), 3.00 (d, J = 4.52 Hz, 3H), 2.28 (s, 3H), 2.00- 2.12 (m, 1H), 0.75-0.95 (m, 4H). |
| | N-(5-((1,2-dimethyl-1H-imidazol-4- | |
| | yl)ethynyl)-8-(methylamino)-2,7- | |
| | naphthyridin-3- | |
| | yl)cyclopropanecarboxamide | |
| Example 1004 | 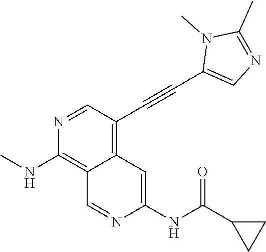 [SMILES: CNc1ncc(C#Cc2cnc(C)n2C)c2cc(NC(=O)C3CC3)ncc12] | LCMS (ESI) m/z 361.2 [M + H]+; 1H NMR (400 MHz, DMSO-d6) δ ppm 11.09 (s, 1H), 9.37 (s, 1H), 8.58 (s, 1H), 8.34 (br d, J = 4.52 Hz, 1H), 8.27 (s, 1H), 7.15 (s, 1H), 3.65 (s, 3H), 3.01 (d, J = 4.02 Hz, 3H), 2.35 (s, 3H), 2.03- 2.10 (m, 1H), 0.77-0.91 (m, 4H). |
| | N-(5-((1,2-dimethyl-1H-imidazol-5- | |
| | yl)ethynyl)-8-(methylamino)-2,7- | |
| | naphthyridin-3- | |
| | yl)cyclopropanecarboxamide | |
| Example 1005 | 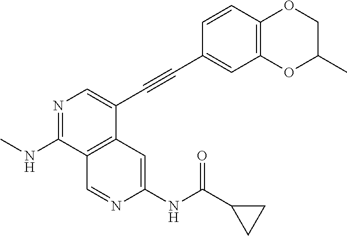 | LCMS (ESI) m/z 414.9 [M + H]+; 1H NMR (400 MHz, DMSO-d6) δ ppm 11.06 (s, 1H), 9.35 (s, 1H), 8.61 (s, 1H), 8.28 (br d, J = 4.4 Hz, 1H), 8.23 (s, 1H), 7.01-7.06 (m, 2H), 6.90 (d, J = 8.8 Hz, 1H), 4.29-4.37 (m, 2H), 3.82-3.90 (m, 1H), 3.00 (d, J = 4.4 Hz, 3H), 2.04-2.11 (m, 1H), 1.30 (d, J = 6.4 Hz, 3H), 0.86-0.91 (m, 2H), 0.80-0.85 (m, 2H). |
| | N-(5-((3-methyl-2,3- | |
| | dihydrobenzo[b][1,4]dioxin-6- | |
| | yl)ethynyl)-8-(methylamino)-2,7- | |
| | naphthyridin-3- | |
| | yl)cyclopropanecarboxamide | |
| Example 1006 | 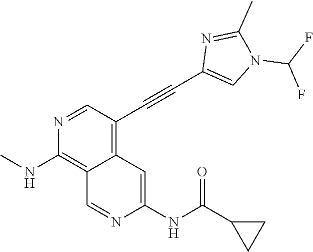 [SMILES: CNc1ncc(C#Cc2cn(C(F)F)c(C)n2)c2cc(NC(=O)C3CC3)ncc12] | LCMS (ESI) m/z 397.1 [M + H]+; 1H NMR (400 MHz, DMSO-d6) δ ppm 11.05 (s, 1H), 9.36 (s, 1H), 8.52 (s, 1H), 8.32 (br d, J = 4.4 Hz, 1H), 8.24 (s, 1H), 7.65-7.94 (m, 2H), 3.01 (d, J = 4.4 Hz, 3H), 2.46 (s, 3H), 2.04-2.09 (m, 1H), 0.81-0.87 (m, 4H). |
| | N-(5-((1-(difluoromethyl)-2-methyl-1H- | |
| | imidazol-4-yl)ethynyl)-8- | |
| | (methylamino)-2,7-naphthyridin-3- | |
| | yl)cyclopropanecarboxamide | |
| Example 1007 | 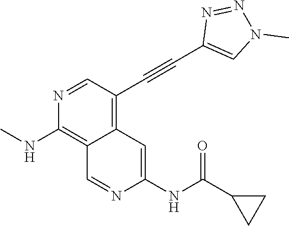 | LCMS (ESI) m/z 348.1 [M + H]+; 1H NMR (400 MHz, DMSO-d6) δ ppm 11.08 (s, 1 H), 9.37 (s, 1 H), 8.54 (s, 1 H), 8.42 (s, 1 H), 8.28-8.31 (m, 1 H), 8.30 (s, 1 H), 4.09 (s, 3 H), 3.02 (d, J = 4.27 Hz, 3 H), 2.06 (br s, 1 H), 0.74-0.96 (m, 4 H). |
| | N-[8-(methylamino)-5-[2-(1- | |
| | methyltriazol-4-yl)ethynyl]-2,7- | |
| | naphthyridin-3- | |
| | yl]cyclopropanecarboxamide | |
| Example 1008 | 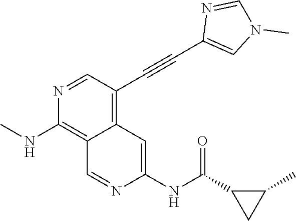 | LCMS (ESI) m/z 361.7 [M + H]+; 1H NMR (400 MHz, DMSO-d6) δ ppm 10.93, (s, 1H) 9.34 (s, 1H), 8.53 (s, 1H), 8.26 (br d, J = 3.6 Hz, 1H), 8.22 (s, 1H), 7.66 (s, 1H), 7.50 (s, 1H), 3.68 (s, 3H), 3.00 (d, J = 4.2 Hz, 3H), 1.27- 1.32 (m, 1H), 1.23 (br s, 1H), 1.12 (d, J = 6.4 Hz, 3H), 0.95- 1.02 (m, 1H), 0.84 (br d, J = 5.2 Hz, 1H). |
| | (1S,2R)-2-methyl-N-(5-((1-methyl-1H- | |
| | imidazol-4-yl)ethynyl)-8- | |
| | (methylamino)-2,7-naphthyridin-3- | |
| | yl)cyclopropane-1-carboxamide | |
| Example 1009 | 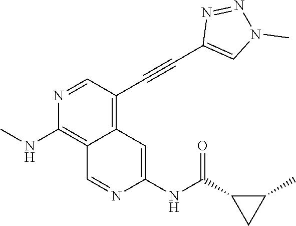 | LCMS (ESI) m/z 362.1 [M + H]+; 1H NMR (400 MHz, DMSO-d6) δ ppm 11.38 (br s, 1H), 10.72 (br s, 1H), 9.81 (s, 1H), 8.67 (s, 1H), 8.52 (s, 1H), 8.13 (s, 1H), 4.12 (s, 3H), 3.17 (br s, 3H), 2.12 (br d, J = 6.27 Hz, 1H), 1.34 (br d, J = 6.27 Hz, 1H), 1.11 (br d, J = 5.77 Hz, 3H), 1.04 (br s, 1H), 0.87 (br s, 1H) |
| | (1S,2R)-N-(5-(5-(2- | |
| | (difluoromethyl)morpholino) | |
| | benzo[d]oxazol-2-yl)-8-(methylamino)- | |
| | 2,7-naphthyridin-3-yl)-2-methyl- | |
| | cyclopropane-1-carboxamide | |
| Example 1010 | 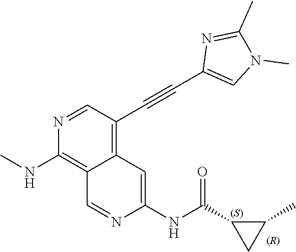 | LCMS (ESI) m/z 375.1 [M + H]+; Chiral purity: 98.3%; 1H NMR (400 MHz, DMSO-d6) δ ppm 10.91 (s, 1H), 9.34 (s, 1H), 8.51 (s, 1H), 8.23 (br d, J = 4.50 Hz, 1H), 8.20 (s, 1H), 7.40 (s, 1H), 3.57 (s, 3H), 3.00 (d, J = 4.25 Hz, 3H), 2.29 (s, 3H), 2.08 (td, J = 8.16, 5.44 Hz, 1H), 1.26-1.34 (m, 1H), 1.13 (d, J = 6.13 Hz, 3H), 0.98 (td, J = 7.97, 3.81 Hz, 1H), 0.80-0.89 (m, 1H). |
| | (1S,2R)-N-(5-((1,2-dimethyl-1H- | |
| | imidazol-4-y)ethynyl)-8- | |
| | (methylamino)-2,7-naphthyridin-3-yl)- | |
| | 2-methylcyclopropane-1-carboxamide | |
| Example 1011 | 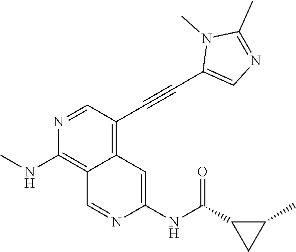 | LCMS (ESI) m/z 374.9 [M + H]+; Chiral purity: 100.0%; 1H NMR (400 MHz, DMSO-d6) δ ppm 10.98 (s, 1H), 9.36 (s, 1H), 8.61 (s, 1H), 8.32 (br d, J = 4.4 Hz, 1H), 8.26 (s, 1H), 7.15 (s, 1H), 3.68 (s, 3H), 3.01 (d, J = 4.3 Hz, 3H), 2.35 (s, 3 H), 2.03-2.14 (m, 1H), 1.24-1.37 (m, 1H), 1.13 (d, J = 6.1 Hz, 3H), 0.93-1.06 (m, 1H), 0.79-0.89 (m, 1H). |
| | 1S,2R)-N-(5-((1,2-dimethyl-1H- | |
| | imidazol-5-yl)ethynyl)-8- | |
| | (methylamino)-2,7-naphthyridin-3-yl)- | |
| | 2-methylcyclopropane-1-carboxamide | |
| Example 1012 | 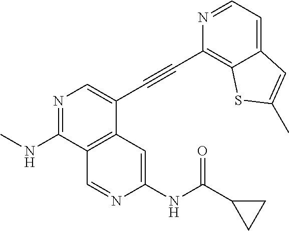 | LCMS (ESI) m/z 414.1 [M + H]+; 1H NMR (400 MHz, DMSO-d6) δ ppm 11.13 (s, 1H), 9.41 (s, 1H), 8.68 (s, 1H), 8.53 (br d, J = 5.3 Hz, 1H), 8.41-8.46 (m, 2H), 7.70 (d, J = 5.5 Hz, 1H), 7.31 (d, J = 1.0 Hz, 1H), 3.05 (d, J = 4.3 Hz, 3H), 2.67 (s, 3H), 2.08 (s, 1H), 0.84-0.90 (m, 4H). |
| | N-(8-(methylamino)-5-((2- | |
| | methylthieno[2,3-c]pyridin-7- | |
| | yl)ethynyl)-2,7-naphthyridin-3- | |
| | yl)cyclopropanecarboxamide | |
| Example 1013 | 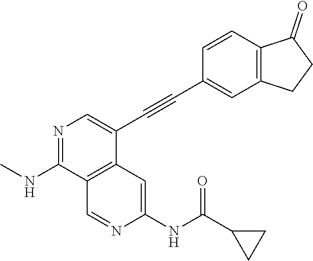 [SMILES: CNc1ncc(C#Cc2ccc3c(c2)CCC3=O)c2cc(NC(=O)C3CC3)ncc12] | LCMS (ESI) m/z 397.1 [M + H]+; 1H NMR (400 MHz, DMSO-d6) δ ppm 11.14 (s, 1H), 9.38 (s, 1H), 8.69 (s, 1H), 8.45 (br d, J = 4.8 Hz, 1H), 8.34 (s, 1H), 7.77 (s, 1H), 7.68 (d, J = 7.8 Hz, 1H), 7.55 (d, J = 8.3 Hz, 1H), 3.09- 3.19 (m, 2H), 3.00-3.07 (m, 3H), 2.64-2.70 (m, 2H), 2.08 (br d, J = 5.0 Hz, 1H), 0.82-0.94 (m, 4H). |
| | N-(8-(methylamino)-5-((1-oxo-2,3- | |
| | dihydro-1H-inden-5-yl)ethynyl)-2,7- | |
| | naphthyridin-3- | |
| | yl)cyclopropanecarboxamide | |
| Example 1014 | 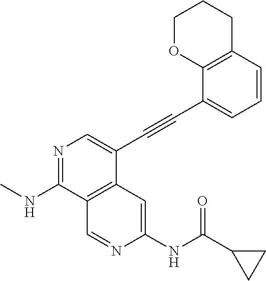 [SMILES: CNc1ncc(C#Cc2cccc3c2OCCC3)c2cc(NC(=O)C3CC3)ncc12] | LCMS (ESI) m/z 399.0 [M + H]+; 1H NMR (400 MHz, DMSO-d6) δ ppm 11.04 (s, 1H), 9.36 (br s, 1H), 8.65 (s, 1H), 8.28 (br d, J = 4.5 Hz, 1H), 8.22 (s, 1H), 7.28 (d, J = 7.5 Hz, 1H), 7.07 (d, J = 7.0 Hz, 1H), 6.83 (t, J = 7.7 Hz, 1H), 4.21-4.35 (m, 2H), 2.96- 3.03 (m, 3H), 2.77 (t, J = 6.4 Hz, 2H), 2.07 (br s, 1H), 1.90-2.01 (m, 2H), 0.80-0.90 (m, 4H). |
| | N-(5-(chroman-8-ylethynyl)-8- | |
| | (methylamino)-2,7-naphthyridin-3- | |
| | yl)cyclopropanecarboxamide | |
| Example 1015 | 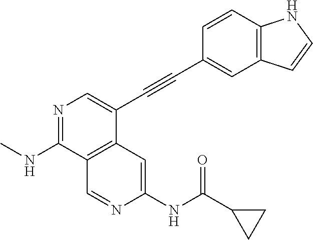 [SMILES: CNc1ncc(C#Cc2ccc3[nH]ccc3c2)c2cc(NC(=O)C3CC3)ncc12] | LCMS (ESI) m/z 382.1 [M + H]+; 1H NMR (400 MHz, DMSO-d6) δ ppm 11.30 (br s, 1H), 11.06 (s, 1H), 9.36 (s, 1H), 8.69 (s, 1H), 8.18-8.35 (m, 2H), 7.79 (s, 1H), 7.39-7.50 (m, 2H), 7.27 (m, 1H), 6.46 (br s, 1H), 3.01 (d, J = 4.3 Hz, 3H), 1.99-2.13 (m, 1H), 0.80-0.93 (m, 4H). |
| | N-(5-((1H-indol-5-yl)ethynyl)-8- | |
| | (methylamino)-2,7-naphthyridin-3- | |
| | yl)cyclopropanecarboxamide | |
| Example 1016 | 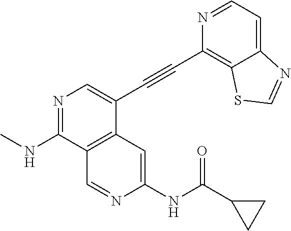 [SMILES: CNc1ncc(C#Cc2nccc3ncsc23)c2cc(NC(=O)C3CC3)ncc12] | LCMS (ESI) m/z 401.0 [M + H]+; 1H NMR (400 MHz, DMSO-d6) δ ppm 11.13 (s, 1H), 9.79 (s, 1H), 9.40 (s, 1H), 8.69 (d, J = 5.5 Hz, 1H), 8.64 (s, 1H), 8.56 (br d, J = 4.5 Hz, 1H), 8.44 (s, 1H), 8.10 (d, J = 5.5 Hz, 1H), 3.05 (d, J = 4.3 Hz, 3H), 2.08 (br s, 1H), 0.83- 0.90 (m, 4H). |
| | N-(8-(methylamino)-5-(thiazolo[5,4- | |
| | c]pyridin-4-ylethynyl)-2,7- | |
| | naphthyridin-3- | |
| | yl)cyclopropanecarboxamide | |
| Example 1017 | 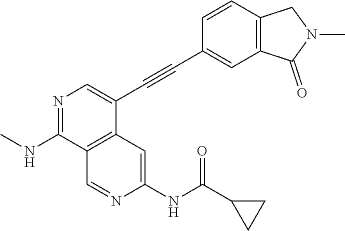 | LCMS (ESI) m/z 412.2 [M + H]+; 1H NMR (400 MHz, DMSO-d6) δ ppm 11.11 (s, 1H), 9.38 (s, 1H), 8.67 (s, 1H), 8.37 (br d, J = 4.3 Hz, 1H), 8.32 (s, 1H), 7.71-7.78 (m, 2H), 7.64-7.69 (m, 1H), 4.51 (s, 2H), 3.09 (s, 3H), 3.00- 3.04 (m, 3H), 2.02-2.14 (m, 1H), 0.88-0.93 (m, 2H), 0.82- 0.87 (m, 2H). |
| | N-(5-((2-methyl-3-oxoisoindolin-5- | |
| | yl)ethynyl)-8-(methylamino)-2,7- | |
| | naphthyridin-3- | |
| | yl)cyclopropanecarboxamide | |
| Example 1018 | 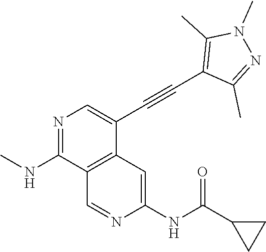 [SMILES: CNc1ncc(C#Cc2c(C)nn(C)c2C)c2cc(NC(=O)C3CC3)ncc12] | LCMS (ESI) m/z 375.1 [M + H]+; 1H NMR (400 MHz, DMSO-d6) δ ppm 11.48 (s, 1H), 10.62 (br s, 1H), 9.74-9.80 (m, 1H), 8.77 (s, 1H), 7.94 (s, 1H), 3.70 (s, 3H), 3.18 (br s, 3H), 2.38 (s, 3H), 2.25 (s, 3H), 2.10 (br t, J = 5.4 Hz, 1H), 0.86-0.90 (m, 4H). |
| | N-(8-(methylamino)-5-((1,3,5-trimethyl- | |
| | 1H-pyrazol-4-yl)ethynyl)-2,7- | |
| | naphthyridin-3- | |
| | yl)cyclopropanecarboxamide | |
| Example 1019 | 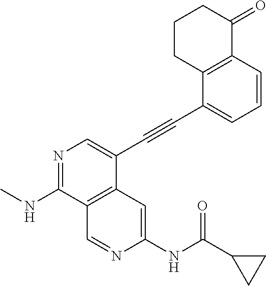 [SMILES: CNc1ncc(C#Cc2cccc3c2CCCC3=O)c2cc(NC(=O)C3CC3)ncc12] | LCMS (ESI) m/z 411.1 [M + H]+; 1H NMR (400 MHz, DMSO-d6) δ ppm 11.11 (s, 1H), 9.38 (s, 1H), 8.70 (s, 1H), 8.38 (br d, J = 4.6 Hz, 1H), 8.33 (s, 1H), 7.88 (m, 1H), 7.77 (m, 1H), 7.41 (t, J = 7.8 Hz, 1H), 3.26-3.29 (m, 2H), 3.01-3.04 (m, 3H), 2.63-2.67 (m, 2H), 2.11-2.17 (m, 2H), 2.06-2.10 (m, 1H), 0.84-0.89 (m, 4H). |
| | N-(8-(methylamino)-5-((5-oxo-5,6,7,8- | |
| | tetrahydronaphthalen-1-yl)ethynyl)- | |
| | 2,7-naphthyridin-3- | |
| | yl)cyclopropanecarboxamide | |
| Example 1020 | 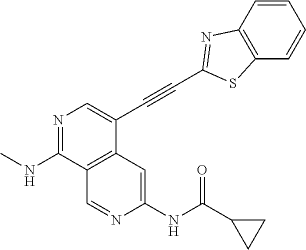 [SMILES: CNc1ncc(C#Cc2nc3ccccc3s2)c2cc(NC(=O)C3CC3)ncc12] | LCMS (ESI) m/z 400.0 [M + H]+; 1H NMR (400 MHz, DMSO-d6) δ ppm 11.16 (s, 1H), 9.41 (s, 1H), 8.64 (br d, J = 4.64 Hz, 1H), 8.59 (s, 1H), 8.48 (s, 1H), 8.16 (d, J = 8.64 Hz, 1H), 8.07 (d, J = 8.88 Hz, 1H), 7.51- 7.62 (m, 2H), 3.05 (d, J = 4.24 Hz, 3H), 2.07-2.11 (m, 1H), 0.83-0.91 (m, 4H). |
| | N-(5-(benzo[d]thiazol-2-ylethynyl)-8- | |
| | (methylamino)-2,7-naphthyridin-3- | |
| | yl)cyclopropanecarboxamide | |
| Example 1021 | 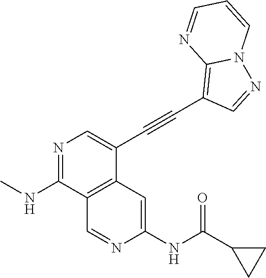 [SMILES: CNc1ncc(C#Cc2cnn3cccnc23)c2cc(NC(=O)C3CC3)ncc12] | LCMS (ESI) m/z: 384.1 [M + H]+; 1H NMR (400 MHz, DMSO-d6) δ ppm 11.07 (s, 1H), 9.37 (s, 1H), 9.22 (dd, J = 8.04, 1.51 Hz, 1H), 8.67 (dd, J = 4.12, 1.51 Hz, 1H), 8.61 (s, 1H), 8.50 (s, 1H), 8.34 (br s, 1H), 8.27 (s, 1H), 7.18 (dd, J = 6.80, 4.14 Hz, 1H), 3.02 (d, J = 4.24 Hz, 3H), 2.03-2.08 (m, 1H), 0.80-0.83 (m, 4H). |
| | N-(8-(methylamino)-5-(pyrazolo[1,5- | |
| | a]pyrimidin-3-ylethynyl)-2,7- | |
| | naphthyridin-3- | |
| | yl)cyclopropanecarboxamide | |

### Example 1022

### Step 1. N-(5-(6-(2-(methoxymethyl)morpholino)-[1,2,4]triazolo[1,5-a]pyridin-2-yl)-8-(methylamino)-2,7-naphthyridin-3-yl)cyclopropanecarboxamide

Synthesis to 4-(2-chloro-[1,2,4]triazolo[1,5-a]pyridin-6-yl)-2-(methoxymethyl)morpholine:

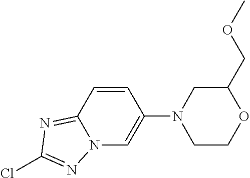 [SMILES: COCC1CN(c2ccc3nc(Cl)nn3c2)CCO1]

A mixture of 2-(methoxymethyl)morpholine (396 mg, 2.37 mmol, 1.1 eq, HCl), 6-bromo-2-chloro-[1,2,4]triazolo[1,5-a]pyridine (500 mg, 2.15 mmol, 1 eq), Pd₂(dba)₃ (393 mg, 0.43 mmol, 0.2 eq), t-BuONa (620 mg, 6.45 mmol, 3 eq) and SPhos (353 mg, 0.86 mmol, 0.4 eq) in dioxane (1 mL) was degassed and purged with N₂ for 3 times. The mixture was stirred at 100° C. for 2 hr under N₂ atmosphere. The residue was diluted with H₂O (30 mL) and extracted with EtOAc (30 mL×3). The combined organic layers were washed with brine (30 mL×2), dried over Na₂SO₄, filtered and concentrated under reduced pressure to give compound 4-(2-chloro-[1,2,4]triazolo[1,5-a]pyridin-6-yl)-2-(methoxymethyl)morpholine (200 mg, 27.96%) as a yellow solid. LCMS (ESI) m/z 283.1 [M+H]⁺.

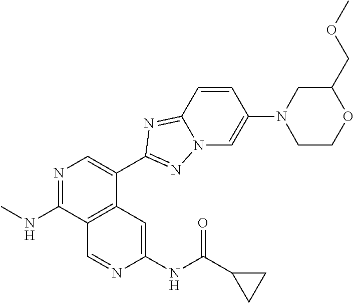

### Step 2. N-(5-(6-(2-(methoxymethyl)morpholino)-[1,2,4]triazolo[1,5-a]pyridin-2-yl)-8-(methylamino)-2,7-naphthyridin-3-yl)cyclopropanecarboxamide

A mixture of 4-(2-chloro-[1,2,4]triazolo[1,5-a]pyridin-6-yl)-2-(methoxymethyl)morpholine (140 mg, 0.49 mmol, 1 eq), N-[8-(methylamino)-5-(4,4,5,5-tetramethyl-1,3,2-dioxaborolan-2-yl)-2,7-naphthyridin-3-yl]cyclopropanecarboxamide (729 mg, 0.59 mmol, 1.2 eq), XPhos (70.82 mg, 0.14 mmol, 0.3 eq), Cs₂CO₃ (484 mg, 1.49 mmol, 3 eq) and Xantphos-Pd-G₄ (95.3 mg, 0.09 mmol, 0.2 eq) in dioxane (1 mL) and H₂O (0.1 mL) was degassed and purged with N₂ for 3 times. The mixture was stirred at 110° C. for 2 hr under N₂ atmosphere. The residue was diluted with NaHCO₃ solution (30 mL) and extracted with EtOAc (30 mL×3). The combined organic layers were washed with brine (30 mL×2), dried over Na₂SO₄, filtered and concentrated under reduced pressure to give a residue. The residue was purified by flash chromatography (ISCO®; 12 g SepaFlash® Silica Flash Column, petroleum ether/EtOAc with EtOAc from 0-400%, 35 mL/min, 254 nm) to give compound N-[5-[6-[2-(methoxymethyl)morpholin-4-yl]imidazo[1,2-a]pyridin-2-yl]-8-(methylamino)-2,7-naphthyridin-3-yl]cyclopropanecarboxamide (105 mg, 42.53%) as a brown solid. LCMS (ESI) m/z 489.3 [M+H]⁺. ¹H NMR (400 MHz, CD₃OD) (5 ppm, 9.58 (s, 1H), 9.23 (s, 1H), 8.76 (s, 1H), 8.44 (s, 1H), 7.63 (s, 2H), 4.02-4.09 (m, 1H), 3.77-3.89 (m, 2H), 3.50-3.58 (m, 3H), 3.47 (br d, J=12.05 Hz, 1H), 3.41 (s, 3H), 3.12 (s, 3H), 2.84 (td, J=11.54, 3.26 Hz, 1H), 2.63 (t, J=11.04 Hz, 1H), 1.93-2.02 (m, 1H), 1.02-1.09 (m, 2H), 0.92-0.98 (m, 2H).

### Examples 1023-1056 and 1114

Each compound in Table 32 was prepared using a similar experimental procedure to prepare Example 1022, step 2, using N-(8-(methylamino)-5-(4,4,5,5-tetramethyl-1,3,2-dioxaborolan-2-yl)-2,7-naphthyridin-3-yl)cyclopropanecarboxamide as the common intermediate and appropriate aryl halides.

TABLE 32

| Example | Structure and Name | MS and ¹H NMR |
|---|---|---|
| Example 1023 | 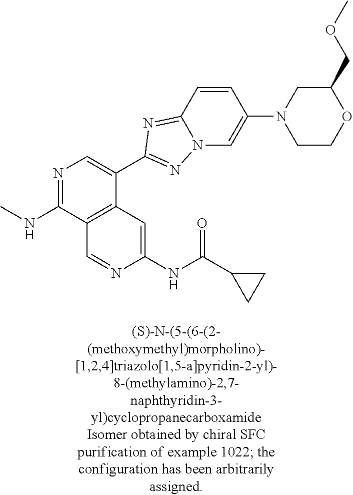 | LCMS (ESI) m/z 489.2 [M + H]⁺, LCMS purity: 100%, HPLC purity: 97.49%, CHIRAL HPLC purity: 99.46%, ¹H NMR (400 MHz, CD₃OD) δ ppm, 9.58 (s, 1H), 9.23 (s, 1H), 8.74 (s, 1H), 8.43 (s, 1H), 7.63 (s, 2H), 4.05 (br d, J = 10.04 Hz, 1H), 3.78-3.90 (m, 2H), 3.51-3.58 (m, 3H), 3.46 (br d, J = 12.05 Hz, 1H), 3.41 (s, 3H), 3.12 (s, 3H), 2.79-2.89 (m, 1H), 2.63 (t, J = 11.04 Hz, 1H), 1.92-2.02 (m, 1H), 1.02-1.09 (m, 2H), 0.93-0.99 (m, 2H). |
| Example 1024 | 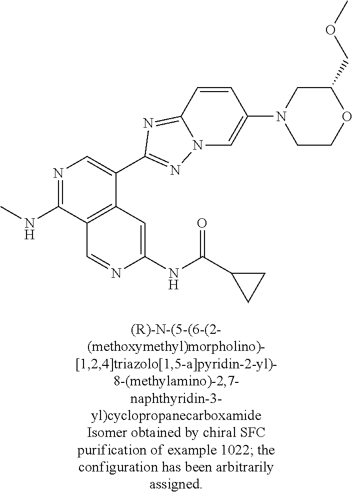 | LCMS (ESI) m/z 489.2 [M + H]⁺, LCMS purity: 100%, HPLC purity: 96.75%, CHIRAL HPLC purity: 100%, ¹H NMR (400 MHz, CD₃OD) δ ppm, 9.58 (s, 1H), 9.23 (s, 1H), 8.74 (s, 1H), 8.43 (s, 1H), 7.63 (s, 2H), 4.05 (br d, J = 10.04 Hz, 1H), 3.78-3.90 (m, 2H), 3.51-3.58 (m, 3H), 3.46 (br d, J = 12.05 Hz, 1H), 3.41 (s, 3H), 3.12 (s, 3H), 2.79-2.89 (m, 1H), 2.63 (t, J = 11.04 Hz, 1H), 1.92-2.02 (m, 1H), 1.02-1.09 (m, 2H), 0.93-0.99 (m, 2H) |
| Example 1025 | 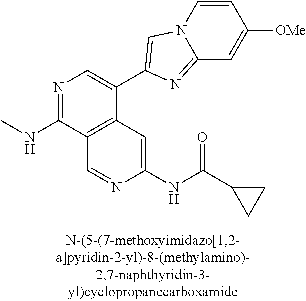 [SMILES: CNc1ncc(-c2cn3ccc(OC)cc3n2)c2cc(NC(=O)C3CC3)ncc12] | LCMS (ESI) m/z 389.1 [M + H]⁺; ¹H NMR (400 MHz, DMSO-d₆) δ ppm 11.49 (br s, 1H), 9.77 (br s, 1H), 8.82- 8.98 (m, 1H), 8.53 (s, 1H), 8.41 (s, 1H), 8.11 (s, 1H), 7.11-7.37 (m, 1H), 7.26 (br d, J = 2.4 Hz, 2H), 4.04 (s, 3H), 3.19 (br d, J = 4.0 Hz, 3H), 2.09 (br s, 1H), 0.77-0.91 (m, 4H). |
| Example 1026 | 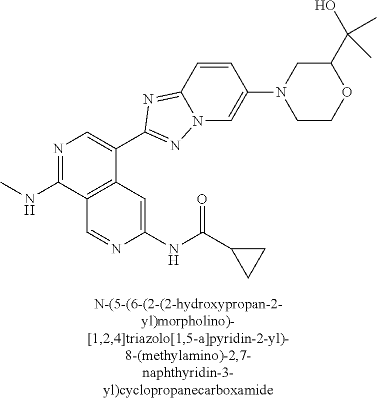 [SMILES: CNc1ncc(-c2nc3ccc(N4CCOC(C(C)(C)O)C4)cn3n2)c2cc(NC(=O)C3CC3)ncc12] | LCMS (ESI) m/z 503.3 [M + H]⁺; ¹H NMR (400 MHz, CD₃OD) δ ppm 9.58 (s, 1H), 9.25 (s, 1H), 8.76 (s, 1H), 8.47 (s, 1H), 7.67 (s, 2H), 4.06- 4.15 (m, 1H), 3.77-3.87 (m, 1H), 3.66 (br d, J = 12 Hz, 1H), 3.44- 3.51 (m, 2H), 3.13 (s, 3H), 2.79- 2.89 (m, 1H), 2.70 (t, J = 12 Hz, 1H), 1.94-2.02 (m, 1H), 1.29 (s, 3H), 1.27 (s, 3H), 1.02-1.07 (m, 2H), 0.92-0.97 (m, 2H). |
| Example 1027 | 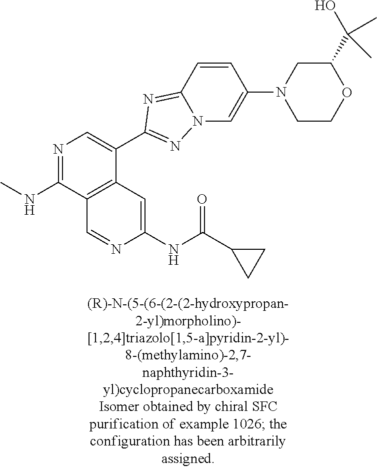 [SMILES: CNc1ncc(-c2nc3ccc(N4CCO[C@@H](C(C)(C)O)C4)cn3n2)c2cc(NC(=O)C3CC3)ncc12] | LCMS (ESI) m/z 503.3 [M + H]⁺, ¹H NMR (400 MHz, CD₃OD) δ ppm 9.56 (s, 1H), 9.23 (s, 1H), 8.75 (s, 1H), 8.45 (s, 1H), 7.65 (s, 2H), 4.10 (br d, J = 12.0 Hz, 1H), 3.76-3.85 (m, 1H), 3.65 (br d, J = 12.0 Hz, 1H), 3.43-3.49 (m, 2H), 3.12 (s, 3H), 2.78-2.88 (m, 1H), 2.68 (t, J = 12.0 Hz, 1H), 1.94-2.01 (m, 1H), 1.28 (s, 3H), 1.26 (s, 3H), 1.01-1.08 (m, 2H), 0.94 (br dd, J = 8.0, 3.26 Hz, 2H). |
| Example 1028 | 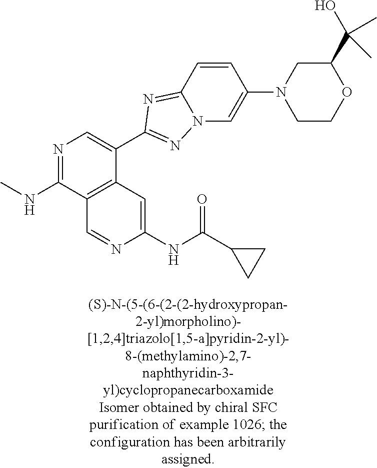 [SMILES: CNc1ncc(-c2nc3ccc(N4CCO[C@H](C(C)(C)O)C4)cn3n2)c2cc(NC(=O)C3CC3)ncc12] | LCMS (ESI) m/z 503.3 [M + H]⁺, ¹H NMR (400 MHz, CD₃OD) δ ppm 9.56 (s, 1H), 9.22 (s, 1H), 8.75 (s, 1H), 8.43 (s, 1H), 7.64 (s, 2H), 4.09 (br d, J = 8.8 Hz, 1H), 3.76-3.83 (m, 1H), 3.64 (br d, J = 12.0 Hz, 1H), 3.45 (br d, J = 10.8 Hz, 2H), 3.12 (s, 3H), 2.78-2.88 (m, 1H), 2.82 (td, J = 12.0, 3.26 Hz, 1H). 1.93-2.01 (m, 1H), 1.28 (s, 3H), 1.26 (s, 3H), 1.01- 1.08 (m, 2H), 0.91-0.97 (m, 2H). |
| Example 1029 | 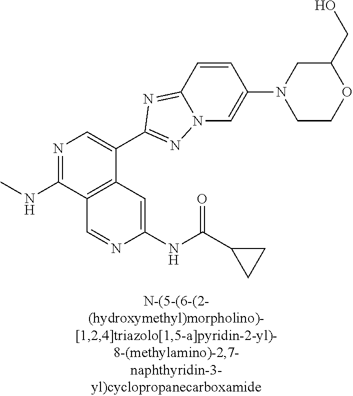 [SMILES: CNc1ncc(-c2nc3ccc(N4CCOC(CO)C4)cn3n2)c2cc(NC(=O)C3CC3)ncc12] | LCMS (ESI) m/z 475.1 [M + H]⁺; ¹H NMR (400 MHz, CD₃OD) δ ppm 9.58 (s, 1H), 9.25 (s, 1H), 8.77 (s, 1H), 8.46 (s, 1H), 7.62-7.71 (m, 2H), 4.08 (dd, J = 11.42, 1.88 Hz, 1H), 3.84 (td, J = 11.42, 2.51 Hz, 1H), 3.76 (ddd, J = 10.16, 5.14, 2.51 Hz, 1H), 3.64-3.70 (m, 2H), 3.59 (br d, J = 11.29 Hz, 1H), 3.49 (br d, J = 11.04 Hz, 1H), 3.13 (s, 3H), 2.87 (td, J = 11.67, 3.26 Hz, 1H), 2.64 (t, J = 10.92 Hz, IH), 1.95-2.01 (m, 1H), 1.01-1.08 (m, 2H), 0.95 (dq, J = 7.34, 3.74 Hz, 2H). |
| Example 1030 | 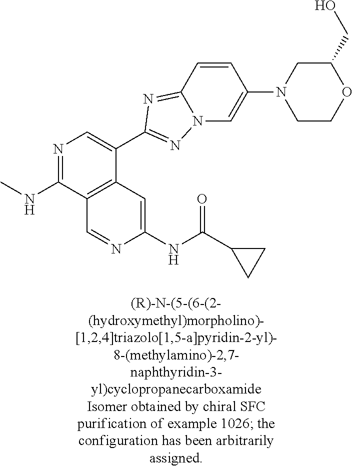 [SMILES: CNc1ncc(-c2nc3ccc(N4CCO[C@@H](CO)C4)cn3n2)c2cc(NC(=O)C3CC3)ncc12] | LCMS (ESI) m/z 475.1 [M + H]⁺, ¹H NMR (400 MHz, CD₃OD) δ ppm 9.57 (s, 1H), 9.22 (br s, 1H), 8.74 (s, 1H), 8.43 (s, 1H), 7.63 (s, 2H), 4.03- 4.13 (m, 1H), 3.83 (td, J = 11.44, 2.64 Hz, 1H), 3.75 (ddd, J = 7.65, 5.12, 2.42 Hz, 1H), 3.63-3.71 (m, 2H), 3.57 (br d, J = 11.22 Hz, 1H), 3.47 (br d, J = 11.66 Hz, 1H), 3.11 (s, 3H), 2.85 (td, J = 11.55, 3.08 Hz, 1H), 2.62 (t, J = 10.89 Hz, 1H), 1.98 (br d, J = 4.40 Hz, IH), 1.02-1.09 (m, 2H), 0.92-0.98 (m, 2H). |
| Example 1031 | 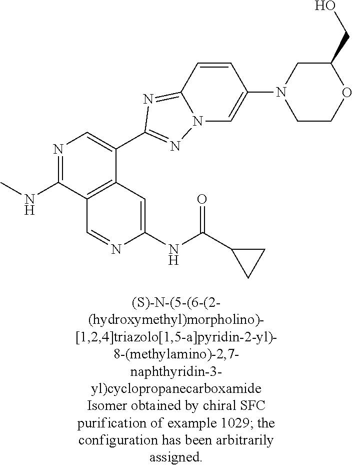 [SMILES: CNc1ncc(-c2nc3ccc(N4CCO[C@H](CO)C4)cn3n2)c2cc(NC(=O)C3CC3)ncc12] | LCMS (ESI) m/z 475.1 [M + H]⁺, ¹H NMR (400 MHz, CD₃OD) δ ppm 9.57 (s, 1H), 9.22 (br s, 1H), 8.74 (s, 1H), 8.43 (s, 1H), 7.63 (s, 2H), 4.03- 4.13 (m, 1H), 3.83 (td, J = 11.44, 2.64 Hz, 1H), 3.75 (ddd, J = 7.65, 5.12, 2.42 Hz, 1H), 3.63-3.71 (m, 2H), 3.57 (br d, J = 11.22 Hz, 1H), 3.47 (br d, J = 11.66 Hz, 1H), 3.11 (s, 3H), 2.85 (td, J = 11.55, 3.08 Hz, 1H), 2.62 (t, J = 10.89 Hz, 1H), 1.98 (br d, J = 4.40 Hz, IH), 1.02-1.09 (m, 2H), 0.92-0.98 (m, 2H). |
| Example 1114 | 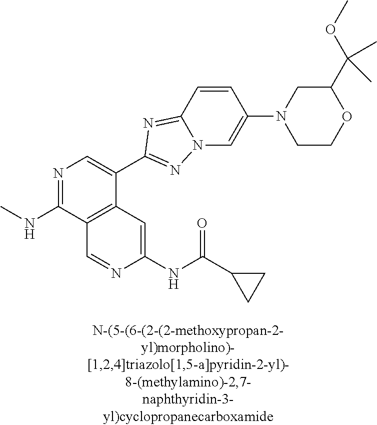 [SMILES: CNc1ncc(-c2nc3ccc(N4CCOC(C(C)(C)OC)C4)cn3n2)c2cc(NC(=O)C3CC3)ncc12] | LCMS (ESI) m/z 517.4 [M + H]⁺, ¹H NMR (400 MHz, CD₃OD) δ ppm 9.57 (s, 1H), 9.23 (s, 1H), 8.75 (s, 1H), 8.44 (s, 1H), 7.63 (d, J = 0.88 Hz, 2H), 4.04-4.11 (m, 1H), 3.78 (br d, J = 2.40 Hz, 1H), 3.55-3.65 (m, 2H), 3.44 (br d, J = 12.04 Hz, 1H), 3.29 (s, 3H), 3.12 (s, 3H), 2.81 (td, J = 12, 3.31 Hz, 1H), 2.67 (t, J = 12 Hz, 1H), 1.87-2.07 (m, 1H), 1.26 (d, J = 12.88 Hz, 6H), 1.01-1.09 (m, 2H), 0.94 (s, 2 H). |
| Example 1032 | 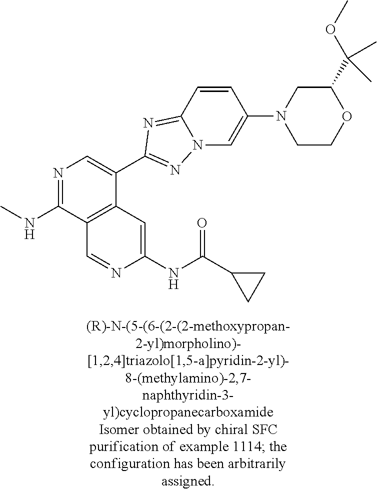 [SMILES: CNc1ncc(-c2nc3ccc(N4CCO[C@@H](C(C)(C)OC)C4)cn3n2)c2cc(NC(=O)C3CC3)ncc12] | LCMS (ESI) m/z 517.4 [M + H]⁺, ¹H NMR (400 MHz, CD₃OD) δ ppm 9.57 (s, 1H), 9.23 (s, 1H), 8.75 (s, 1H), 8.44 (s, 1H), 7.63 (d, J = 0.88 Hz, 2H), 4.04-4.11 (m, 1H), 3.78 (br d, J = 2.40 Hz, 1H), 3.55-3.65 (m, 2H), 3.44 (br d, J = 12.04 Hz, 1H), 3.29 (s, 3H), 3.12 (s, 3H), 2.81 (td, J = 12, 3.31 Hz, 1H), 2.67 (t, J = 12 Hz, 1H), 1.87-2.07 (m, 1H), 1.26 (d, J = 12.88 Hz, 6H), 1.01-1.09 (m, 2H), 0.94 (s, 2 H). |
| Example 1033 | 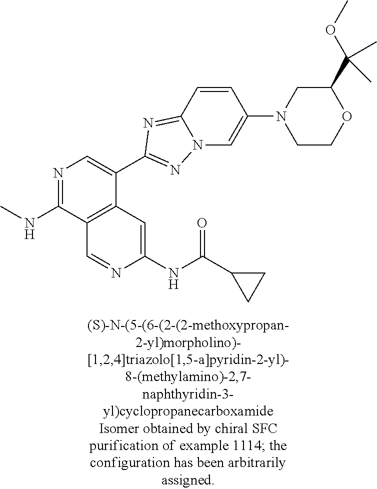 [SMILES: CNc1ncc(-c2nc3ccc(N4CCO[C@H](C(C)(C)OC)C4)cn3n2)c2cc(NC(=O)C3CC3)ncc12] | LCMS (ESI) m/z 517.4 [M + H]⁺, ¹H NMR (400 MHz, CD₃OD) δ ppm 9.58 (s, 1H), 9.24 (s, 1H), 8.76 (s, 1H), 8.45 (s, 1H), 7.64 (s, 2H), 4.08 (dd, J = 11.13, 50 Hz, 1H), 3.79 (br d, J = 2.13 Hz, 1H), 3.57-3.65 (m, 2H), 3.45 (br d, J = 11.76 Hz, 1H), 3.30 (s, 3H), 3.13 (s, 3H), 2.76-2.88 (m, 1H), 2.68 (t, J = 10.94 Hz, 1H), 1.94-2.04 (m, 1H), 1.27 (d, J = 14.01 Hz, 6H), 1.03-1.10 (m, 2H), 0.96 (br d, J = 7.75 Hz, 2H). |
| Example 1034 | 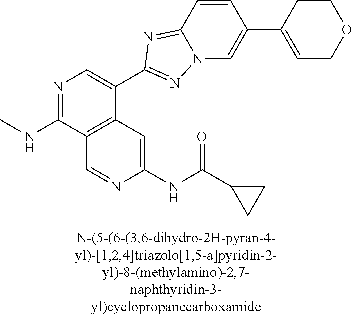 [SMILES: CNc1ncc(-c2nc3ccc(C4=CCOCC4)cn3n2)c2cc(NC(=O)C3CC3)ncc12] | LCMS (ESI) m/z 441.9 [M + H]⁺, ¹H NMR (400 MHz, DMSO-d₆) δ ppm 10.94 (s, 1H), 9.45 (s, 1H), 9.40 (s, 1H), 8.89 (s, 1H), 8.78 (s, 1H), 8.34- 8.26 (m, 1H), 7.92 (dd, J = 9.5, 1.8 Hz, 1H), 7.84-7.78 (m, 1H), 6.50 (br s, 1H), 4.27 (br d, J = 2.8 Hz, 2H), 3.86 (t, J = 5.4 Hz, 2H), 3.05 (d, J = 4.5 Hz, 3H), 2.55 (br s, 2H), 2.09- 2.02 (m, 1H), 0.86-0.80 (m, 4H). |
| Example 1035 | 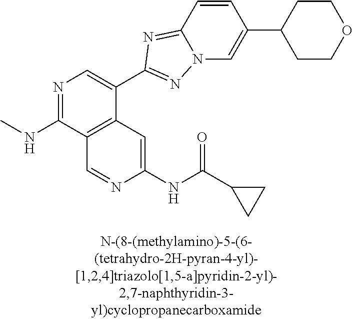 [SMILES: CNc1ncc(-c2nc3ccc(C4CCOCC4)cn3n2)c2cc(NC(=O)C3CC3)ncc12] | LCMS (ESI) m/z 444.2 [M + H]⁺; ¹H NMR (400 MHz, DMSO-d₆) δ ppm 10.95 (s, 1H), 9.43 (d, J = 15.3 Hz, 2H), 8.77 (d, J = 9.5 Hz, 2H), 8.36- 8.23 (m, 1H), 7.80 (d, J = 9.0 Hz, 1H), 7.69 (dd, ] = 9.3, 1.5 Hz, 1H), 4.01-3.96 (m, 2H), 3.51-3.43 (m, 2H), 3.05 (d, J = 4.3 Hz, 3H), 3.01- 2.94 (m, 1H), 2.10-2.04 (m, 1H), 1.82-1.75 (m, 4H), 0.88-0.81 (m, 4H). |
| Example 1036 | 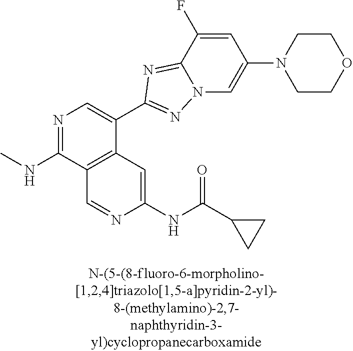 [SMILES: CNc1ncc(-c2nc3c(F)cc(N4CCOCC4)cn3n2)c2cc(NC(=O)C3CC3)ncc12] | LCMS (ESI) m/z 463.1 [M + H]⁺; ¹H NMR (400 MHz, DMSO-d₆) δ ppm 10.93 (s, 1H), 9.39 (d, J = 12.4 Hz, 2H), 8.74 (s, 1H), 8.29 (br d, J = 4.4 Hz, 1H), 8.26 (s, 1H), 7.76 (br d, J = 13.1 Hz, 1H), 3.83-3.73 (m, 4H), 3.21-3.14 (m, 4H), 3.05 (d, J = 4.1 Hz, 3H), 2.13-2.02 (m, 1H), 0.89- 0.80 (m, 4H). |
| Example 1037 | 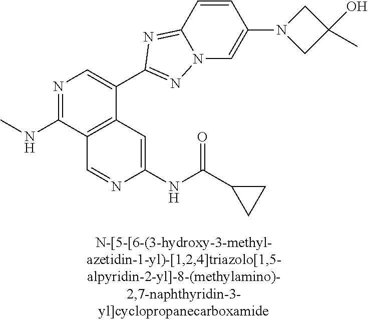 | LCMS (ESI) m/z 445.3 [M + H]⁺; ¹H NMR (400 MHz, DMSO-d₆) δ ppm 10.90 (s, 1H), 9.39 (d, J = 5.9 Hz, 2H), 8.70 (s, 1H), 8.21 (br d, J = 4.5 Hz, 1H), 8.05 (s, 1H), 7.69 (d, J = 9.5 Hz, 1H), 7.14 (dd, J = 9.4, 1.6 Hz, 1H), 5.83-5.34 (m, 1H), 3.84 (br d, J = 7.3 Hz, 2H), 3.68 (br d, J = 7.3 Hz, 2H), 3.04 (br d, J = 4.1 Hz, 3H), 2.10- 2.02 (m, 1H), 1.48 (s, 3H), 0.88- 0.79 (m, 4H). |
| Example 1038 | 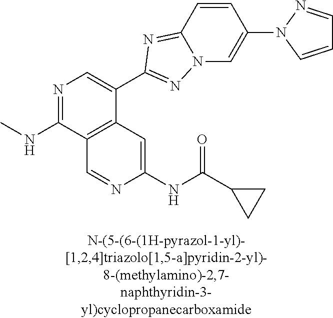 [SMILES: CNc1ncc(-c2nc3ccc(-n4cccn4)cn3n2)c2cc(NC(=O)C3CC3)ncc12] | LCMS (ESI) m/z 426.2 [M + H]⁺; ¹H NMR (400 MHz, CDCl₃) δ ppm 9.70 (s, 1H), 9.05 (s, 1H), 8.99 (s, 1H), 8.89 (s, 1H), 8.58 (br s, 1H), 7.87- 7.94 (m, 2H), 7.85-7.76 (m, 1H), 7.73 (d, J = 1.00 Hz, 1H), 6.49 (s, 1H), 5.91-5.75 (m, 1H), 3.19 (br s, 3H), 1.60-1.56 (m, 1H), 1.13-1.07 (m, 2H), 0.86 (br dd, J = 7.53, 3.01 Hz, 2H). |
| Example 1039 | 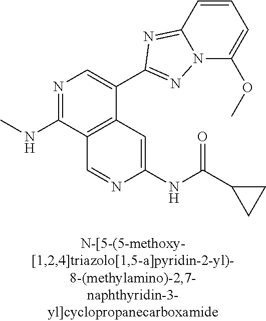 [SMILES: CNc1ncc(-c2nc3cccc(OC)n3n2)c2cc(NC(=O)C3CC3)ncc12] | LCMS (ESI) m/z 390.1 [M + H]⁺; ¹H NMR (400 MHz, CD₃OD) δ ppm 9.32 (s, 1H), 9.25 (s, 1H), 8.62 (s, 1H), 8.26-8.17 (m, 1H), 7.77-7.67 (m, 1H), 7.43 (d, J = 8.8 Hz, 1H), 6.67 (d, J = 7.8 Hz, 1H), 4.25 (s, 3H), 3.14 (s, 3H), 1.99-1.87 (m, 1H), 1.02-0.97 (m, 2H), 0.91 (dt, J = 7.8, 3.1 Hz, 2H). |
| Example 1040 | 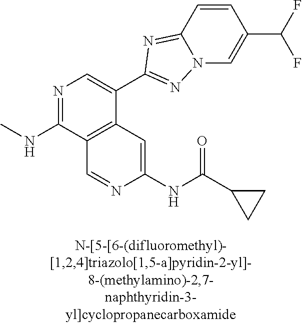 [SMILES: CNc1ncc(-c2nc3ccc(C(F)F)cn3n2)c2cc(NC(=O)C3CC3)ncc12] | LCMS (ESI) m/z 410.0 [M + H]⁺; ¹H NMR (400 MHz, DMSO-d₆) δ ppm 10.97 (s, 1H), 9.56 (s, 1H), 9.42 (s, 1H), 9.31 (s, 1H), 8.89 (s, 1H), 8.36 (br d, J = 4.5 Hz, 1H), 7.99 (d, J = 9.3 Hz, 1H), 7.85 (dd, J = 9.4, 1.1 Hz, 1H), 7.44-7.03 (m, 1H), 3.06 (d, J = 4.5 Hz, 3H), 2.13-2.05 (m, 1H), 0.90-0.81 (m, 4H). |
| Example 1041 | 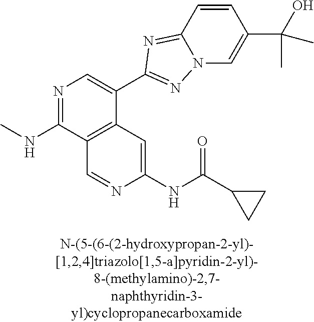 [SMILES: CNc1ncc(-c2nc3ccc(C(C)(C)O)cn3n2)c2cc(NC(=O)C3CC3)ncc12] | LCMS (ESI) m/z 418.2 [M + H]⁺, ¹H NMR (400 MHz, CD₃OD) δ ppm, 9.88 (br s, 1H), 9.50 (br s, 1H), 9.06 (br s, 1H), 8.44 (br d, J = 6.8 Hz, 1H), 7.94 (br s, 1H), 7.82 (br s, 1H), 2.02 (br s, 1H), 1.66 (br d, J = 5.6 Hz, 6H), 0.97-1.13 (m, 4H). |
| Example 1042 | 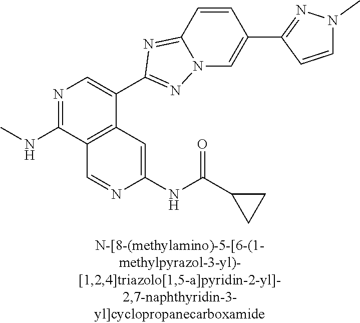 | LCMS (ESI) m/z 440.1 [M + H]⁺, ¹H NMR (400 MHz, DMSO-d₆) δ ppm 10.95 (s, 1H), 9.52 (s, 1H), 9.41 (s, 1H), 9.28 (s, 1H), 8.82 (s, 1H), 8.33 (b rs, 1H), 8.14 (br d, J = 9.2 Hz, 1H), 7.88 (br d, J = 9.2 Hz, IH), 7.82 (s, 1H), 6.96 (s, 1H), 3.93 (s, 3H), 3.06 (b rd, J = 4.0 Hz, 3H), 2.08 (br s, 1H), 0.82-0.90 (m, 4H). |
| Example 1043 | 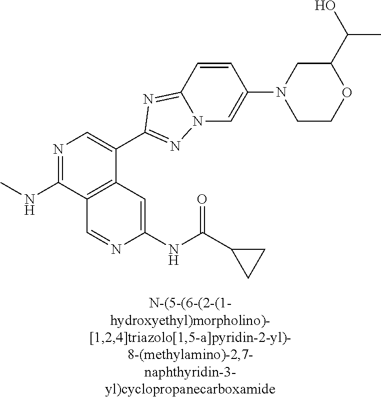 [SMILES: CNc1ncc(-c2nc3ccc(N4CCOC(C(C)O)C4)cn3n2)c2cc(NC(=O)C3CC3)ncc12] | LCMS (ESI) m/z 489.3 [M + H]⁺; ¹H NMR (400 MHz, CD₃OD) δ ppm 9.54-9.64 (m, 1H), 9.24 (s, 1 H), 8.75 (s, 1H), 8.46 (br s, 1H), 7.65 (d, J = 1.76 Hz, 2H), 4.63 (s, 1H), 4.02- 4.15 (m, 1H), 3.74-3.87 (m, 2H), 3.46-3.56 (m, 2H), 3.12 (s, 3H), 2.80-2.89 (m, 1H) 2.64-2.75 (m, 1H), 1.98 (br t, J = 4.52 Hz, 1H), 1.25-1.28 (m, 3H), 1.02-1.08 (m, 2H), 0.95 (td, J = 7.47, 4.14 Hz, 2H). |
| Example 1044 | 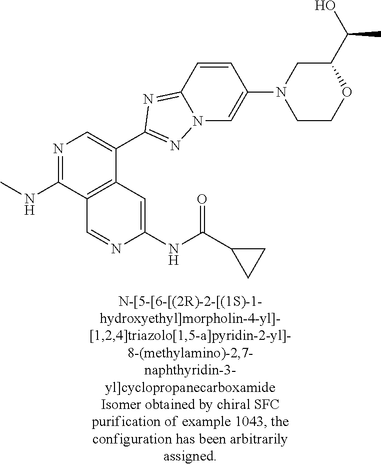 [SMILES: CNc1ncc(-c2nc3ccc(N4CCO[C@@H]([C@H](C)O)C4)cn3n2)c2cc(NC(=O)C3CC3)ncc12] | LCMS (ESI) m/z 489.3 [M + H]⁺, LCMS purity: 91.5%, HPLC purity: 92.0%, CHIRAL HPLC purity: 95.6%, ¹H NMR (400 MHz, CD₃OD) δ ppm 9.50-9.68 (m, 1H), 9.26 (br s, 1H), 8.76 (s, 1H), 8.47 (s, 1H), 7.67 (s, 2H), 4.08 (br dd, J = 11.2, 2.00 Hz, 1H), 3.72-3.84 (m, 3H), 3.45- 3.50 (m, 2H), 3.13 (s, 3H), 2.86 (br d, J = 3.2 Hz, 1H), 2.64 (br t, J = 10.8 Hz, 1H), 1.94 (s, 1H), 1.29 (s, 3H), 0.95 (br d, J = 4.0 Hz, 2H), 0.88 (br s, 2H). |
| Example 1045 | 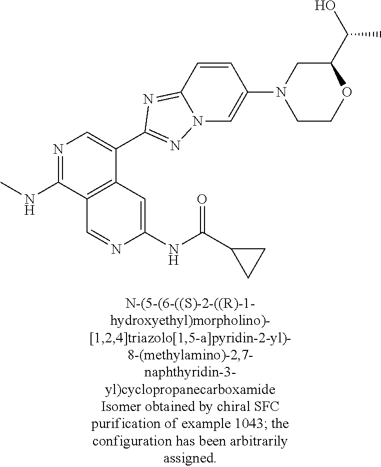 [SMILES: CNc1ncc(-c2nc3ccc(N4CCO[C@H]([C@@H](C)O)C4)cn3n2)c2cc(NC(=O)C3CC3)ncc12] | LCMS (ESI) m/2 489.3 [M + H]⁺, LCMS purity: 96.4%, HPLC purity: 95.3%, CHIRAL HPLC purity: 91.3%, ¹H NMR (400 MHz, CD₃OD) δ ppm 9.59 (s, 1H), 9.26 (s, 1H), 8.77 (s, 1H), 8.48 (s, 1H), 7.63-7.72 (m, 2H), 4.11 (dd, J = 11.6, 2.4 Hz, 1H), 3.76-3.88 (m, 2H), 3.45-3.56 (m, 3H), 3.13 (s, 3H), 2.81-2.93 (m, 1H), 2.72 (t, J = 11.6 Hz, 1H), 1.95- 2.01 (m, 1H), 1.27 (d, J = 6.4 Hz, 3H), 1.02-1.08 (m, 2H), 0.95 (br dd, J = 7.6, 3.81Hz, 2H). |
| Example 1046 | 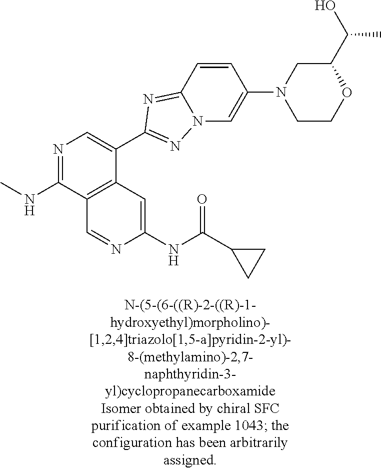 [SMILES: CNc1ncc(-c2nc3ccc(N4CCO[C@@H]([C@@H](C)O)C4)cn3n2)c2cc(NC(=O)C3CC3)ncc12] | LCMS (ESI) m/z 489.3 [M + H]⁺, LCMS purity: 97.7%, HPLC purity: 93.5%, CHIRAL HPLC purity: 100%, ¹H NMR (400 MHz, CD₃OD) δ ppm 9.59 (s, 1H), 9.26 (s, 1H), 8.76 (s, 1H), 8.47 (s, 1H), 7.68 (s, 2H), 4.08 (dd, J = 11.6, 2.0 Hz, 1H), 3.71- 3.87 (m, 3H), 3.48 (dt, J = 6.8, 3.2 Hz, 2H), 3.14 (s, 3H), 2.86 (td, J = 11.6, 3.2 Hz, 1H), 2.61-2.69 (m, 1H), 1.95- 2.02 (m, 1H), 1.29 (s, 3H), 1.03- 1.07 (m, 2H), 0.95 (dt, J = 7.6, 3.2 Hz, 2H). |
| Example 1047 | 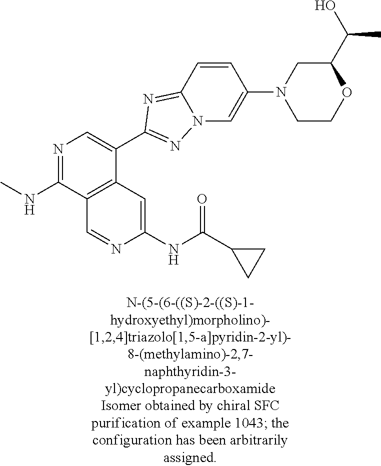 [SMILES: CNc1ncc(-c2nc3ccc(N4CCO[C@H]([C@H](C)O)C4)cn3n2)c2cc(NC(=O)C3CC3)ncc12] | LCMS (ESI) m/z 489.3 [M + H]⁺, LCMS purity: 96.6%, HPLC purity: 93.2%, CHIRAL HPLC purity: 87.2%, ¹H NMR (400 MHz, CD₃OD) δ ppm 9.59 (s, 1H), 9.26 (s, 1H), 8.75 (s, 1H), 8.47 (s, 1H), 7.62-7.72 (m, 2H), 4.11 (br dd, J = 11.2, 2.0 Hz, 1H), 3.77-3.87 (m, 2H), 3.44-3.58 (m, 3H), 3.13 (s, 3H), 2.86 (td, J = 11.6, 3.2 Hz, 1H), 2.72 (br t, J = 11.6 Hz, 1H), 1.95-2.02 (m, 1H), 1.27 (br d, J = 6.4 Hz, 3H), 1.02-1.09 (m, 2H), 0.95 (td, J = 7.2, 3.6 Hz, 2H). |
| Example 1048 | 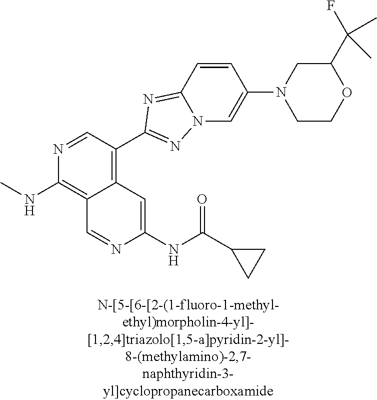 [SMILES: CNc1ncc(-c2nc3ccc(N4CCOC(C(C)(C)F)C4)cn3n2)c2cc(NC(=O)C3CC3)ncc12] | LCMS (ESI) m/z 505.3 [M + H]⁺, ¹H NMR (400 MHz, DMSO-d₆) δ ppm 10.94 (br s, 1H), 9.33-9.45 (m, 2H), 8.70 (br s, 1H), 8.48 (br s, 1H), 8.16- 8.39 (m, 1H), 7.74 (br s, 2H), 4.04 (br s, 1H), 3.69-3.78 (m, 1H), 3.51-3.64 (m, 3H), 3.04 (br s, 3H), 2.64-2.86 (m, 2H), 2.06 (br d, J = 3.6 Hz, 1H), 1.34-1.47 (m, 6H), 0.83 (br s, 4H). |
| Example 1049 | 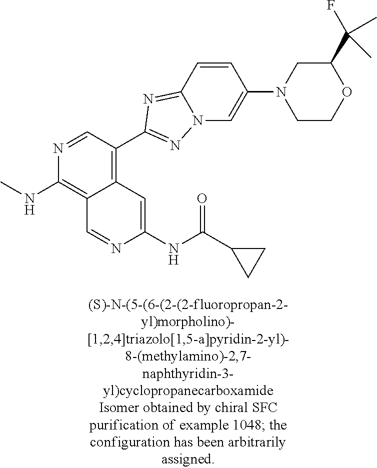 [SMILES: CNc1ncc(-c2nc3ccc(N4CCO[C@H](C(C)(C)F)C4)cn3n2)c2cc(NC(=O)C3CC3)ncc12] | LCMS (ESI) m/z 505.3 [M + H]⁺, LCMS purity: 95.9%, HPLC purity: 96.9%, CHIRAL HPLC purity: 100%, ¹H NMR (400 MHz, DMSO- d₆) δ ppm 10.94 (s, 1H), 9.39 (d, J = 6.8 Hz, 2H), 8.70 (s, 1H), 8.47 (s, 1H), 8.26 (br s, 1H), 7.74 (s, 2H), 4.05 (dd, J = 10.8, 2.0 Hz, 1H), 3.68- 3.78 (m, 1H), 3.54-3.64 (m, 3H), 3.01-3.06 (m, 3H), 2.60-2.68 (m, 2H), 1.99-2.13 (m, 1H), 1.36-1.43 (m, 6H), 0.81-0.86 (m, 4H). |
| Example 1050 | 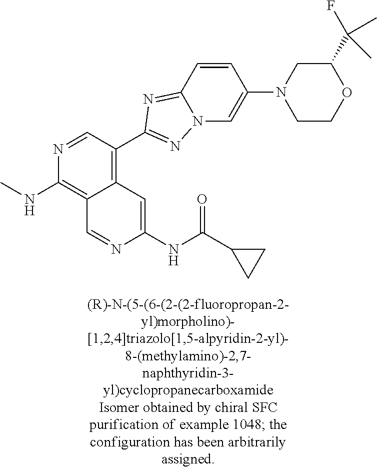 [SMILES: CNc1ncc(-c2nc3ccc(N4CCO[C@@H](C(C)(C)F)C4)cn3n2)c2cc(NC(=O)C3CC3)ncc12] | LCMS (ESI) m/z 505.3 [M + H]⁺, LCMS purity: 99.4%, HPLC purity: 98.6%, CHIRAL HPLC purity: 94.0%, ¹H NMR (400 MHz, DMSO- d₆) δ ppm 10.93 (s, 1H), 9.39 (d, J = 6.4 Hz, 2H), 8.70 (s, 1H), 8.47 (s, 1H), 8.25 (br d, J = 4.4 Hz, 1H), 7.74 (s, 2H), 4.05 (dd, J = 11.2, 2.4 Hz, 1H), 3.68-3.76 (m, 1H), 3.53-3.64 (m, 3H), 3.02-3.06 (m, 3H), 2.58- 2.71 (m, 2H), 2.02-2.10 (m, 1H), 1.36-1.43 (m, 6H), 0.80-0.85 (m, 4H). |
| Example 1051 | 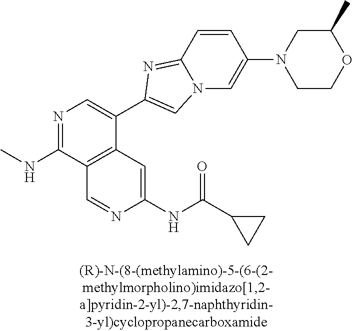 | LCMS (ESI) m/z 458.3 [M + H]⁺, ¹H NMR (400 MHz, DMSO-d₆) δ ppm 10.94 (s, 1H), 9.37 (s, 1H), 8.90 (s, 1H), 8.29 (s, 1H), 8.05 (s, 1H), 8.01 (b rd, J = 4.8 Hz, 1H), 7.97 (s, 1H), 7.49 (d, J = 9.6 Hz, 1H), 7.27 (dd, J = 9.6, 2.0 Hz, 1H), 3.93 (br d, J = 10.0 Hz, 1H), 3.64-3.75 (m, 2H), 3.47 (br d, J = 11.6 Hz, 1H), 3.40 (br d, J = 11.2 Hz, 1H), 3.01 (d, J = 4.4 Hz, 3H), 2.58- 2.68 (m, 1H), 2.32 (t, J = 10.8 Hz, 1H), 2.04 (quin, J = 6.0 Hz, 1H), 1.17 (d, J = 6.4 Hz, 3H), 0.77-0.84 (m, 4H). |
| Example 1052 | 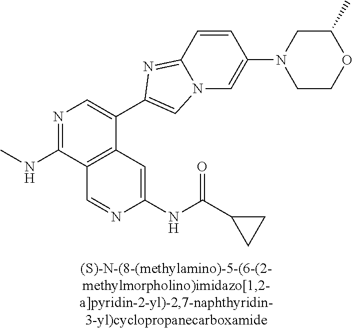 | LCMS (ESI) m/z 458.3 [M + H]⁺, ¹H NMR (400 MHz, DMSO-d₆) δ ppm, 10.95 (s, 1H), 9.37 (s, 1H), 8.90 (8, 1H), 8.29 (s, 1H), 8.05 (d, J = 2.0 Hz, 1H), 7.99-8.04 (m, 1H), 7.97 (s, 1H), 7.49 (d, J = 10.0 Hz, 1H), 7.27 (dd, J = 10.0, 2.0 Hz, 1H), 3.93 (br d, J = 11.2 Hz, 1H), 3.65-3.74 (m, 2H), 3.47 (br d, J = 11.2 Hz, 1H), 3.40 (br d, J = 11.6 Hz, 1H), 3.01 (d, J = 4.0 Hz, 3H), 2.58-2.70 (m, 1H), 2.32 (t, J = 10.8 Hz, 1H), 2.04 (quin, J = 6.0 Hz, 1H), 1.16 (d, J = 6.0 Hz, 3H), 0.79-0.85 (m, 4H). |
| Example 1053 | 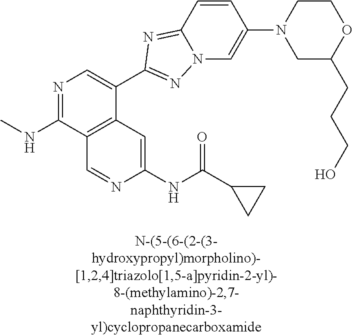 [SMILES: CNc1ncc(-c2nc3ccc(N4CCOC(CCCO)C4)cn3n2)c2cc(NC(=O)C3CC3)ncc12] | LCMS (ESI) m/z 503.3 [M + H]⁺, ¹H NMR (400 MHz, CD₃OD) δ ppm, 9.63 (s, 1H), 9.54 (s, 1H), 8.71 (s, 1H), 8.42 (s, 1H), 8.02 (br d, J = 9.6 Hz, 1H), 7.86 (d, J = 9.6 Hz, 1H), 4.09 (dd, J = 11.6, 2.4 Hz, 1H), 3.84 (td, J = 11.6, 2.4 Hz, 1H), 3.56-3.76 (m, 5H), 3.33 (s, 3H), 2.92-3.03 (m, 1H), 2.68 (t, J = 10.8 Hz, 1H), 1.97-2.10 (m, 1H), 1.75-1.84 (m, 1H), 1.62-1.73 (m, 3H), 0.98-1.14 (m, 4H). |
| Example 1054 | 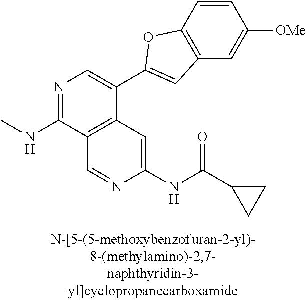 [SMILES: CNc1ncc(-c2cc3cc(OC)ccc3o2)c2cc(NC(=O)C3CC3)ncc12] | LCMS (ESI) m/z 389.2 [M + H]⁺, ¹H NMR (400 MHz, CD₃OD) δ ppm 9.24 (s, 1H), 8.91 (s, 1H), 8.41 (s, 1H), 7.43 (d, J = 8.8 Hz, 1H), 7.15 (d, J = 2.8 Hz, 1H), 7.01 (s, 1H), 6.88 (dd, J = 8.8, 2.8 Hz, 1H), 3.85 (s, 3H), 3.12 (s, 3H), 1.92-2.00 (m, 1H), 1.00-1.07 (m, 2H), 0.90-0.96 (m, 2H). |
| Example 1055 | 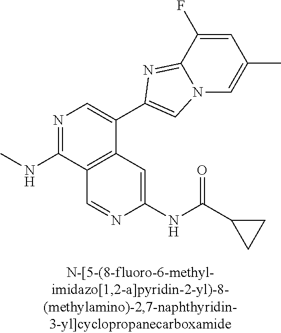 | LCMS (ESI) m/z 391.5 [M + H]⁺, ¹H NMR (400 MHz, DMSO-d₆) δ ppm, 10.96 (s, 1H), 9.3 (s, 1H), 8.88 (s, 1H), 8.40-8.22 (m, 2H), 8.20-7.96 (m, 2H), 7.08 (d, J = 12.0 Hz, 1H), 3.04 (d, J = 4.4 Hz, 3H), 2.32 (s, 3H), 2.12- 1.96 (m, 1H), 0.80 (br d, J = 6.0 Hz, 4H). |
| Example 1056 | 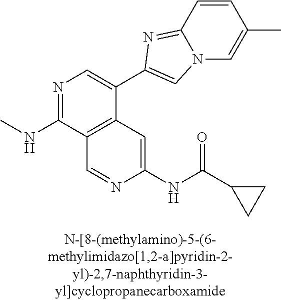 | LCMS (ESI) m/z 372.9 [M + H]⁺, ¹H NMR (400 MHz, DMSO-d₆) δ 10.92- 10.96 (m, 1H), 9.38 (s, 1H), 8.94 (s, 1H), 8.41 (s, IH), 8.34 (s, 1H), 8.02 (s, 2H), 7.51 (d, J = 9.2 Hz, 1H), 7.13 (d d, J = 9.2, 1.6 Hz, 1H), 3.01-3.04 (m, 3H), 2.31 (s, 3H), 2.01-2.09 (m, 1H), 0.78-0.85 (m, 4H). |

### Example 1057

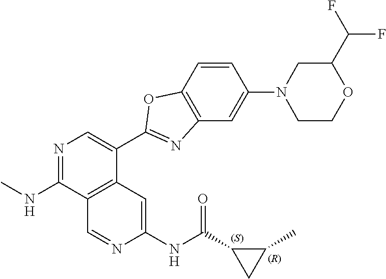

(1S,210-N-(5-(5-(2-(difluoromethyl)morpholino) benzo[d]oxazol-2-yl)-8-(methylamino)-2,7-naphthyridin-3-yl)-2-methylcyclopropane-1-carboxamide

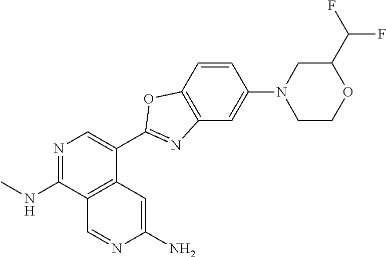 [SMILES: CNc1ncc(-c2nc3cc(N4CCOC(C(F)F)C4)ccc3o2)c2cc(N)ncc12]

Step 1. Synthesis to 4-(5-(2-(difluoromethyl)morpholino)benzo[d]oxazol-2-yl)-N₁-methyl-2,7-naphthyridine-1,6-diamine. A mixture of (Example 329)N-[5-[5-[2-(difluoromethyl)morpholin-4-yl]-1,3-benzoxazol-2-yl]-8-(methylamino)-2,7-naphthyridin-3-yl]cyclopropanecarboxamide (700 mg, 1.42 mmol), NaOH (566 mg, 14.2 mmol) in MeOH/H₂O (3 mL) was degassed and purged with N₂ for 3 times, and then the mixture was stirred at 65° C. for 24 hrs under N₂ atmosphere. The mixture was concentrated in vacuum. The crude product was triturated with water (10 ml) at 25° C. for 10 min. to afford 4-[5-[2-(difluoromethyl)morpholin-4-yl]-1,3-benzoxazol-2-yl]-N₁-methyl-2,7-naphthyridine-1,6-diamine (550 mg, 91.1% yield) as yellow solid; LCMS (ESI) m/z 427.2 [M+H]⁺.

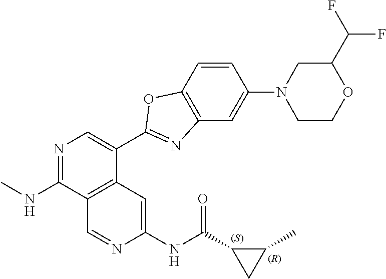

### Step 2. Synthesis of (1S,2R)—N-(5-(5-(2-(difluoromethyl)morpholino)benzo[d]oxazol-2-yl)-8-(methylamino)-2,7-naphthyridin-3-yl)-2-methylcyclopropane-1-carboxamide

To a solution of 4-[5-[2-(difluoromethyl)morpholin-4-yl]-1,3-benzoxazol-2-yl]-N₁-methyl-2,7-naphthyridine-1,6-diamine (212 mg, 499 μmol) and (1S,2R)-2-methylcyclopropanecarboxylic acid (50 mg, 499 μmol) in pyridine (3 mL) was added dropwise POCl₃ (1.50 mmol, 140 μL) at 0° C. under N₂. The mixture was stirred at 25° C. for 1 hr. The mixture was quenched with water (10 ml). The aqueous phase was extracted with ethyl acetate (1.0 mL×3). The combined organic phase was washed with brine (10 mL×1), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=0/1) to afford (1S,2R)—N-[5-[5-[2-(difluoromethyl)morpholin-4-yl]-1,3-benzoxazol-2-yl]-8-(methylamino)-2,7-naphthyridin-3-yl]-2-methyl-cyclopropanecarboxamide (55 mg, 21.7% yield) as yellow solid. LCMS (ESI) m/z 509.3 [M+H]⁺. ¹H NMR (400 MHz, DMSO-d₆) δ ppm 10.95 (s, 1H), 9.60 (s, (m, 3H), 0.87-0.92 (m, 1H), 1.02 (td, J=7.98, 4.07 Hz, 1H).

### Examples 1058-1059

| | | |
|---|---|---|
| Example 1058 | 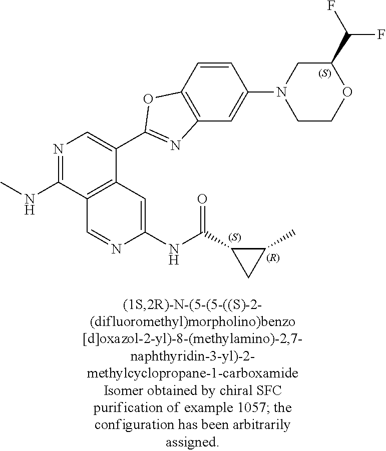 | LCMS (ESI) m/z 509.3 [M + H]⁺; ¹H NMR (400 MHz, DMSO-d₆) δ ppm 10.93 (s, 1H), 9.61 (s, 1H), 9.45 (s, 1H), 8.89 (s, 1H), 8.67 (br d, J = 4.84 Hz, 1H), 7.61 (d, J = 8.80 Hz, 1H), 7.29 (d, J = 2.20 Hz, 1H), 7.10 (s, 1H), 6.15 (d, J = 3.52 Hz, 1H), 4.02- 4.11 (m, 1H), 3.92-4.02 (m, 1H), 3.72-3.82 (m, 1H), 3.60-3.68 (m, 1H), 3.52-3.60 (m, 1H), 3.09 (d, J = 4.18 Hz, 3H), 2.79-2.88 (m, 1H), 2.69-2.76 (m, 1H), 2.05-2.17 (m, 1H), 1.27-1.39 (m, 1H), 1.16 (d, J = 6.16 Hz, 3H), 0.97-1.06 (m, 1H), 0.83-0.94 (m, 1H). |
| Example 1059 | 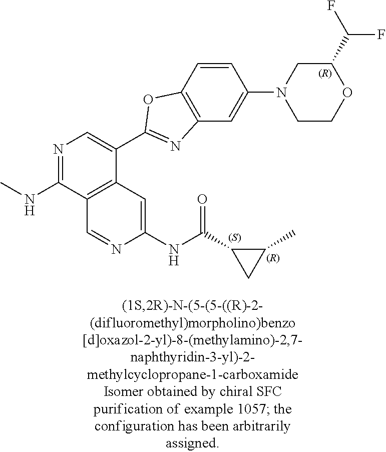 | LCMS (ESI) m/z 509.3 [M + H]⁺; ¹H NMR (400 MHz, DMSO-d₆) δ ppm 10.92 (s, 1H), 9.61 (s, 1H), 9.45 (s, 1H), 8.89 (s, 1H), 8.68 (br d, J = 4.40 Hz, 1H), 7.61 (d, J = 8.80 Hz, 1H), 7.29 (d, J = 2.20 Hz, 1H), 7.10 (dd, J = 8.91, 2.09 Hz, 1H), 5.97-6.33 (m, 1H), 4.06 (br d, J = 11.66 Hz, 1H), 3.91-4.00 (m, 1H), 3.73-3.82 (m, 1H), 3.64 (br d, J = 11.66 Hz, 1H), 3.55 (br d, J = 11.44 Hz, 1H), 3.08 (d, J = 4.18 Hz, 3H), 2.83 (td, J = 11.55, 3.08 Hz, 1H), 2.72 (t, J = 10.89 Hz, 1H), 2.07-2.15 (m, 1H), 1.28-1.37 (m, 1H), 1.16 (d, J = 6.16 Hz, 3H), 1.02 (td, J = 8.14, 4.18 Hz, 1H), 0.85- 0.93 (m, 1H). |

### Examples 994 and 1060-1085, 1114

Each compound in Table 33 was prepared using a similar experimental procedure to prepare Example 1057, step 2, using appropriate N¹-methyl-2,7-naphthyridine-1,6-diamine as the common intermediate and cyclopropanecarboxylic acid substrates.

TABLE 33

| Example | Structure and Name | MS and ¹H NMR |
|---|---|---|
| Example 1060 | 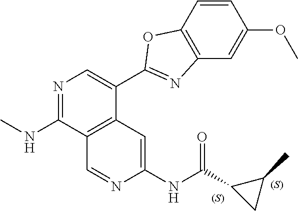 | LCMS (ESI) m/z 404.1 [M + H]⁺; ¹H NMR (400 MHz, DMSO-d₆) δ ppm 10.96 (s, 1H), 9.63 (s, 1H), 9.43 (s, 1H), 8.89 (s, 1H), 8.65 (br d, J = 4.4 Hz, 1H), 7.63 (d, J = 8.8 Hz, 1H), 7.28 (d, J = 2.6 Hz, 1H), 6.96 (dd, J = 8.8, 2.6 Hz, 1H), 3.86 (s, 3H), 3.09 (d, J = 4.5 Hz, 3H), 1.86 (dt, J = 8.1, 4.2 Hz, 1H), 1.29-1.40 (m, 1H), 1.06- 1.17 (m, 4H), 0.67-0.76 (m, 1H). |
| | (1S,2S)-N-(5-(5-methoxybenzo[d]oxazol- | |
| | 2-yl)-8-(methylamino)-2,7- | |
| | naphthyridin-3-yl)-2- | |
| | methylcyclopropane-1-carboxamide | |
| Example 1061 | 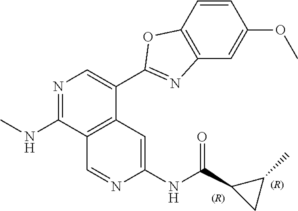 | LCMS (ESI) m/z 404.1 [M + H]⁺; ¹H NMR (400 MHz, DMSO-d₆) δ ppm 11.27 (s, 1H), 10.25 (br s, 1H), 9.58-9.81 (m, 2H), 8.56 (s, 1H), 7.70 (d, J = 8.9 Hz, 1H), 7.33 (d, J = 2.5 Hz, 1H), 7.03 (dd, J = 8.9, 2.5 Hz, 1H), 3.87 (s, 3H), 3.21 (br d, J = 4.1 Hz, 3H), 1.88 (dt, J = 7.9, 4.1 Hz, 1H), 1.26- 1.46 (m, 1H), 1.03-1.17 (m, 4H), 0.66-0.81 (m, 1H) |
| | (1R,2R)-N-(5-(5- | |
| | methoxybenzo[d]oxazol-2-yl)-8- | |
| | (methylamino)-2,7-naphthyridin-3-yl)- | |
| | 2-methylcyclopropane-1-carboxamide | |
| Example 1062 | 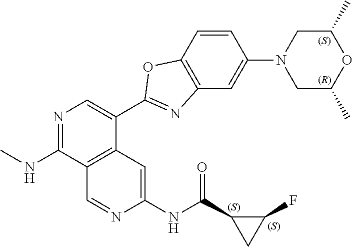 | LCMS (ESI) m/z 491.1 [M + H]⁺; Chiral purity: 100.0%; ¹H NMR (400 MHz, CDCl₃) δ ppm 9.93 (s, 1H), 9.03 (s, 1H), 8.97 (s, 1H), 8.34 (br s, 1H), 7.47 (d, J = 8.8 Hz, 1H), 7.36 (d, J = 2.0 Hz, 1H), 6.99 (dd, J = 8.9, 2.3 Hz, 1H), 6.14-5.92 (m, 1H), 5.01-4.75 (m, 1H), 3.93-3.85 (m, 2H), 3.47 (br d, J = 11.2 Hz, 2H), 3.27 (d, J = 4.8 Hz, 3H), 2.49 (t, J = 11.0 Hz, 2H), 2.07-1.93 (m, 2H), 1.36- 1.32 (m, 1H), 1.30 (d, J = 6.2 Hz, 6H). |
| | (1S,2S)-N-[5-[5-[(2R,6S)-2,6- | |
| | dimethylmorpholin-4-yl]-1,3- | |
| | benzoxazol-2-yl]-8-(methylamino)-2,7- | |
| | naphthyridin-3-yl]-2-fluoro- | |
| | cyclopropanecarboxamide | |
| Example 1063 | 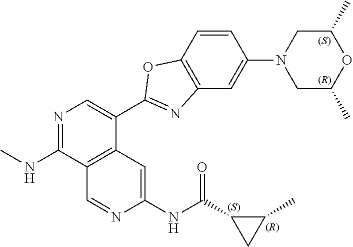 | LCMS (ESI) m/z 487.1 [M + H]⁺; ¹H NMR (400 MHz, DMSO-d₆) δ ppm 10.90 (s, 1H), 9.59 (s, 1H), 9.42 (s, 1H), 8.87 (s, 1H), 8.61 (br d, J = 4.5 Hz, 1H), 7.56 (d, J = 8.9 Hz, 1H), 7.23 (d, J = 2.3 Hz, 1H), 7.06 (dd, J = 9.0, 2.4 Hz, 1H), 3.75 (ddd, J = 10.1, 6.4, 2.2 Hz, 2H), 3.60 (br d, J = 11.8 Hz, 2H), 3.08 (d, J = 4.4 Hz, 3H), 2.31 (t, J = 11.0 Hz, 2H), 2.11 (br d, J = 5.9 Hz, 1H), 1.31 (br d, J = 6.6 Hz, 1H), 1.20-1.13 (m, 9H), 1.01 (td, J = 8.1, 3.9 Hz, 1H), 0.93-0.85 (m, 1H). |
| | (1S,2R)-N-[5-[5-[(2R,6S)-2,6- | |
| | dimethylmorpholin-4-yl]-1,3- | |
| | benzoxazol-2-yl]-8-(methylamino)-2,7- | |
| | naphthyridin-3-yl]-2-methyl- | |
| | cyclopropanecarboxamide | |
| Example 1064 | 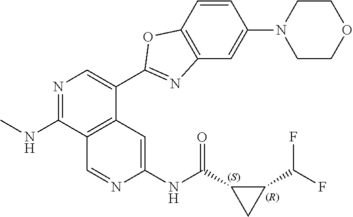 | LCMS (ESI) m/z 495.3 [M + H]⁺; Chiral purity: 99.8%; ¹H NMR (400 MHz, DMSO-d₆) δ ppm 11.26 (s, 1H), 9.62 (s, 1H), 9.45 (s, 1H), 8.89 (s, 1H), 8.74-8.55 (m, 1H), 7.59 (d, J = 9.03 Hz, 1H), 7.24 (d, J = 2.26 Hz, 1H), 7.06 (dd, J = 8.91, 2.38 Hz, 1H), 6.31-5.91 (m, 1H), 3.86-3.72 (m, 4H), 3.19-3.11 (m, 4H), 3.11- 3.05 (m, 3H), 2.46-2.39 (m, 1H), 2.00-1.86 (m, 1H), 1.45- 1.28 (m, 2H). |
| | (1S,2R)-2-(difluoromethyl)-N-(8- | |
| | (methylamino)-5-(5- | |
| | morpholinobenzo[d]oxazol-2-yl)-2,7- | |
| | naphthyridin-3-yl)cyclopropane-1- | |
| | carboxamide | |
| | The configuration has been arbitrarily | |
| | assigned. | |
| Example 1065 | 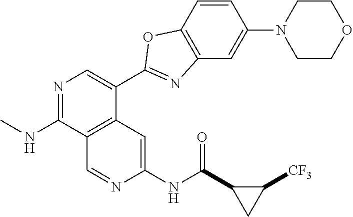 | LCMS (ESI) m/z 513.1 [M + H]⁺; ¹H NMR (400 MHz, DMSO-d₆) δ ppm 11.17 (s, 1H), 9.60 (s, 1H), 9.44 (s, 1H), 8.89 (s, 1H), 8.70- 8.59 (m, 1H), 7.58 (d, J = 8.78 Hz, 1H), 7.22 (d, J = 2.26 Hz, 1H), 7.07 (dd, J = 8.91, 2.38 Hz, 1H), 3.81-3.74 (m, 4H), 3.19- 3.13 (m, 4H), 3.10-3.06 (m, 3H), 2.31-2.23 (m, 1H), 1.65-1.57 (m, 1H), 1.40-1.26 (m, 2H). |
| | N-(8-(methylamino)-5-(5- | |
| | morpholinobenzo[d]oxazol-2-yl)-2,7- | |
| | naphthyridin-3-yl)-2- | |
| | (trifluoromethyl)cyclopropane-1- | |
| | carboxamide | |
| Example 1066 | 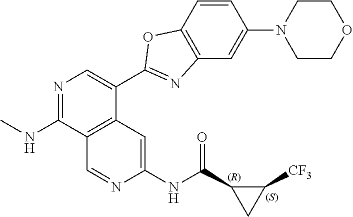 | LCMS (ESI) m/z 513.1 [M + H]⁺; chiral purity: 100.0%; ¹H NMR (400 MHz, DMSO-d₆) δ ppm 11.17 (s, 1H), 9.60 (s, 1H), 9.44 (s, 1H), 8.89 (s, 1H), 8.70-8.59 (m, 1H), 7.58 (d, J = 8.78 Hz, 1H), 7.22 (d, J = 2.26 Hz, 1H), 7.07 (dd, J = 8.91, 2.38 Hz, 1H), 3.81-3.74 (m, 4H), 3.19-3.13 (m, 4H), 3.10-3.06 (m, 3H), 2.31- 2.23 (m, 1H), 1.65-1.57 (m, 1H), 1.40-1.26 (m, 2H). |
| | (1R,2S)-N-(8-(methylamino)-5-(5- | |
| | morpholinobenzo[d]oxazol-2-yl)-2,7- | |
| | naphthyridin-3-yl)-2- | |
| | (trifluoromethyl)cyclopropane-1- | |
| | carboxamide | |
| | Isomer obtained by chiral SFC purification | |
| | of example 1065; the configuration has | |
| | been arbitrarily assigned. | |
| Example 1067 | 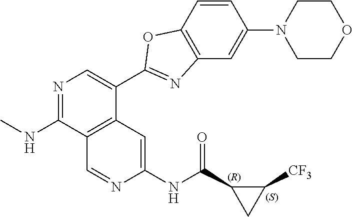 | LCMS (ESI) m/z 513.1 [M + H]⁺; chiral purity: 96.2%; ¹H NMR (400 MHz, DMSO-d₆) δ ppm 11.17 (s, 1H), 9.60 (s, 1H), 9.44 (s, 1H), 8.89 (s, 1H), 8.70-8.59 (m, 1H), 7.58 (d, J = 8.78 Hz, 1H), 7.22 (d, J = 2.26 Hz, 1H), 7.07 (dd, J = 8.91, 2.38 Hz, 1H), 3.81-3.74 (m, 4H), 3.19-3.13 (m, 4H), 3.10-3.06 (m, 3H), 2.31- 2.23 (m, 1H), 1.65-1.57 (m, 1H), 1.40-1.26 (m, 2H). |
| | (1S,2R)-N-(8-(methylamino)-5-(5- | |
| | morpholinobenzo[d]oxazol-2-yl)-2,7- | |
| | naphthyridin-3-yl)-2- | |
| | (trifluoromethyl)cyclopropane-1- | |
| | carboxamide | |
| | Isomer obtained by chiral SFC purification | |
| | of example 1065; the configuration has | |
| | been arbitrarily assigned. | |
| Example 1068 | 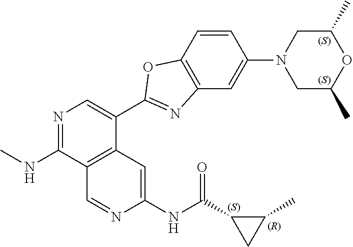 | LCMS (ESI) m/z 487.0 [M + H]⁺; chiral purity: 98.1%; ¹H NMR (400 MHz, DMSO-d₆) δ ppm 10.91 (s, 1H), 9.60 (s, 1H), 9.42 (s, 1H), 8.87 (s, 1H), 8.61 (br d, J = 4.5 Hz, 1H), 7.56 (d, J = 8.8 Hz, 1H), 7.20 (d, J = 2.3 Hz, 1H), 7.03 (dd, J = 9.0, 2.3 Hz, 1H), 4.13-4.05 (m, 2H), 3.20 (dd, J = 11.7, 2.9 Hz, 2H), 3.07 (d, J = 4.3 Hz, 3H), 2.87 (dd, J = 11.5, 6.0 Hz, 2H), 2.10 (td, J = 8.2, 5.5 Hz, 1H), 1.37-1.28 (m, 1H), 1.24 (d, J = 6.5 Hz, 6H), 1.15 (d, J = 6.0 Hz, 3H), 1.01 (td, J = 8.0, 4.0 Hz, 1H), 0.92-0.87 (m, 1H). |
| | (1S,2R)-N-[5-[5-[(2S,6S)-2,6- | |
| | dimethylmorpholin-4-yl]-1,3- | |
| | benzoxazol-2-yl]-8-(methylamino)-2,7- | |
| | naphthyridin-3-yl]-2-methyl- | |
| | cyclopropanecarboxamide | |
| Example 1069 | 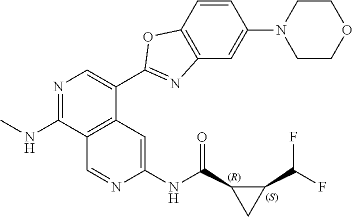 | LCMS (ESI) m/z 495.1 [M + H]⁺; chiral purity: 100.0%; ¹H NMR (400 MHz, DMSO-d₆) δ ppm 11.28 (s, 1H), 9.62 (s, 1H), 9.45 (s, 1H), 8.89 (s, 1H), 8.66 (br d, J = 4.52 Hz, 1H), 7.59 (d, J = 9.03 Hz, 1H), 7.24 (d, J = 2.26 Hz, 1H), 7.06 (dd, J = 9.03, 2.26 Hz, 1H), 6.26-5.90 (m, 1H), 3.83- 3.73 (m, 4H), 3.18-3.12 (m, 4H), 3.08 (d, J = 4.27 Hz, 3H), 2.47- 2.39 (m, 1H), 2.00-1.87 (m, 1H), 1.44-1.28 (m, 2H). |
| | (1R,2S)-2-(difluoromethyl)-N-(8- | |
| | (methylamino)-5-(5- | |
| | morpholinobenzo[d]oxazol-2-yl)-2,7- | |
| | naphthyridin-3-yl)cyclopropane-1- | |
| | carboxamide | |
| | The configuration has been arbitrarily | |
| | assigned. | |
| Example 1070 | 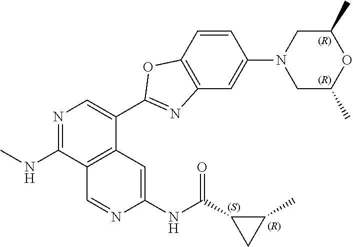 | LCMS (ESI) m/z 487.3 [M + H]⁺; Chiral purity: 100.0%; ¹H NMR (400 MHz, DMSO-d₆) δ ppm 10.64-10.58 (m, 1H), 9.53 (s, 1H), 9.41 (s, 1H), 8.88 (s, 1H), 8.43-8.38 (m, 1H), 7.54 (d, J = 8.8 Hz, 1H), 7.21 (d, J = 2.3 Hz, 1H), 7.02 (dd, J = 9.2, 2.4 Hz, 1H), 4.12 (td, J = 6.1, 3.0 Hz, 2H), 3.23 (br dd, J = 11.4, 2.6 Hz, 3H), 3.16 (s, 1H), 2.90 (dd, J = 11.7, 6.1 Hz, 2H), 2.16-2.08 (m, 1H), 1.35-1.28 (m, 2H), 1.27 (d, J = 6.3 Hz, 6H), 1.18 (d, J = 6.0 Hz, 3H), 1.05-0.98 (m, 1H), 0.94- |
| | (1S,2R)-N-(5-(5-((2R,6R)-2,6- | 0.89 (m, 1H). |
| | dimethylmorpholino)benzo[d]oxazol-2- | |
| | yl)-8-(methylamino)-2,7-naphthyridin- | |
| | 3-yl)-2-methylcyclopropane-1- | |
| | carboxamide | |
| Example 1071 | 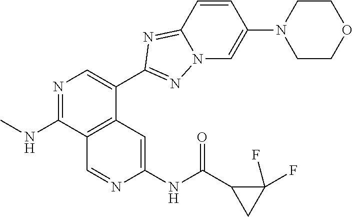 [SMILES: CNc1ncc(-c2nc3ccc(N4CCOCC4)cn3n2)c2cc(NC(=O)C3CC3(F)F)ncc12] | LCMS (ESI) m/z 481.0 [M + H]⁺; ¹H NMR (400 MHz, DMSO-d₆) δ ppm 11.15 (s, 1H), 9.42 (s, 2H), 8.75 (s, 1H), 8.34 (d, J = 1.8 Hz, 1H), 8.29 (br d, J = 4.5 Hz, 1H), 7.77-7.73 (m, 1H), 7.71-7.66 (m, 1H), 3.82-3.75 (m, 4H), 3.18- 3.13 (m, 4H), 3.05 (d, J = 4.0 Hz, 4H), 2.15-1.97 (m, 2H). |
| | 2,2-difluoro-N-(8-(methylamino)-5-(6- | |
| | morpholino-[1,2,4]triazolo[1,5- | |
| | a]pyridin-2-yl)-2,7-naphthyridin-3- | |
| | yl)cyclopropane-1-carboxamide | |
| Example 994 | 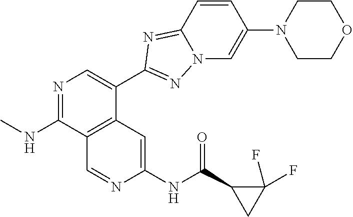 [SMILES: CNc1ncc(-c2nc3ccc(N4CCOCC4)cn3n2)c2cc(NC(=O)[C@@H]3CC3(F)F)ncc12] | LCMS (ESI) m/z 481.0 [M + H]⁺; ¹H NMR (400 MHz, DMSO-d₆) δ ppm 11.15 (s, 1H), 9.42 (s, 2H), 8.75 (s, 1H), 8.34 (d, J = 1.8 Hz, 1H), 8.29 (br d, J = 4.5 Hz, 1H), 7.77-7.73 (m, 1H), 7.71-7.66 (m, 1H), 3.82-3.75 (m, 4H), 3.18- 3.13 (m, 4H), 3.05 (d, J = 4.0 Hz, 4H), 2.15-1.97 (m, 2H). |
| | (1S)-2,2-difluoro-N-(8-(methylamino)-5- | |
| | (6-morpholino-[1,2,4]triazolo[1,5- | |
| | a]pyridin-2-yl)-2,7-naphthyridin-3- | |
| | yl)cyclopropane-1-carboxamide | |
| | Isomer obtained by chiral SFC purification | |
| | of example 1071; the configuration has | |
| | been arbitrarily assigned. | |
| Example 1141 | 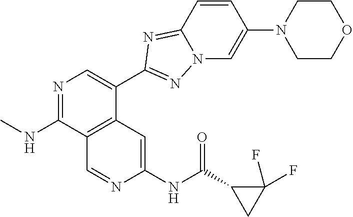 [SMILES: CNc1ncc(-c2nc3ccc(N4CCOCC4)cn3n2)c2cc(NC(=O)[C@H]3CC3(F)F)ncc12] | LCMS (ESI) m/z 481.0 [M + H]⁺; ¹H NMR (400 MHz, DMSO-d₆) δ ppm 11.15 (s, 1H), 9.42 (s, 2H), 8.75 (s, 1H), 8.34 (d, J = 1.8 Hz, 1H), 8.29 (br d, J = 4.5 Hz, 1H), 7.77-7.73 (m, 1H), 7.71-7.66 (m, 1H), 3.82-3.75 (m, 4H), 3.18- 3.13 (m, 4H), 3.05 (d, J = 4.0 Hz, 4H), 2.15-1.97 (m, 2H). |
| | (1R)-2,2-difluoro-N-(8-(methylamino)- | |
| | 5-(6-morpholino-[1,2,4]triazolo[1,5- | |
| | a]pyridin-2-yl)-2,7-naphthyridin-3- | |
| | yl)cyclopropane-1-carboxamide | |
| | Isomer obtained by chiral SFC purification | |
| | of example 1071; the configuration has | |
| | been arbitrarily assigned. | |
| Example 1072 | 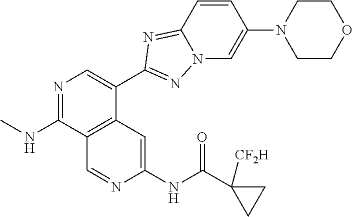 [SMILES: CNc1ncc(-c2nc3ccc(N4CCOCC4)cn3n2)c2cc(NC(=O)C3(C(F)F)CC3)ncc12] | LCMS (ESI) m/z 495.1 [M + H]⁺; ¹H NMR (400 MHz, DMSO-d₆) δ ppm 10.05 (s, 1H), 9.41 (d, J = 2.76 Hz, 2H), 8.76 (s, 1H), 8.35- 8.29 (m, 2H), 7.77-7.72 (m, 1H), 7.71-7.65 (m, 1H), 6.84-6.48 (m, 1H), 3.79-3.76 (m, 4H), 3.17- 3.13 (m, 4H), 3.04 (d, J = 4.52 Hz, 3H), 1.38 (br s, 2H), 1.16- 1.11 (m, 2H). |
| | 1-(difluoromethyl)-N-[8-(methylamino)- | |
| | 5-(6-morpholino-[1,2,4]triazolo[1,5- | |
| | a]pyridin-2-yl)-2,7-naphthyridin-3- | |
| | yl]cyclopropanecarboxamide | |
| Example 1073 | 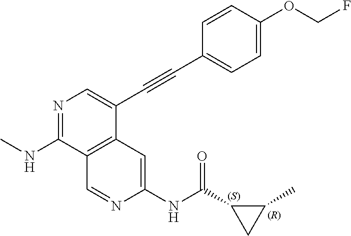 | LCMS (ESI) m/z 405.0 [M + H]⁺; chiral SFC purity: 100%; ¹H NMR (400 MHz, DMSO-d₆) δ ppm 10.98 (s, 1H), 9.36 (s, 1H), 8.67 (s, 1H), 8.31 (br d, J = 4.8 Hz, 1H), 8.26 (s, 1H), 7.63-7.53 (m, 2H), 7.18 (d, J = 8.5 Hz, 2H), 6.04-5.78 (m, 2H), 3.01 (d, J = 4.5 Hz, 3H), 2.09 (td, J = 8.2, 5.5 Hz, 1H), 1.35-1.25 (m, 1H), 1.15 (d, J = 6.0 Hz, 3H), 1.00 (td, J = 8.1, 3.9 Hz, 1H), 0.89-0.83 (m, 1H). |
| | (1S,2R)-N-[5-[2-[4- | |
| | (fluoromethoxy)phenyl]ethynyl]-8- | |
| | (methylamino)-2,7-naphthyridin-3-yl]- | |
| | 2-methyl-cyclopropanecarboxamide | |
| Example 1074 | 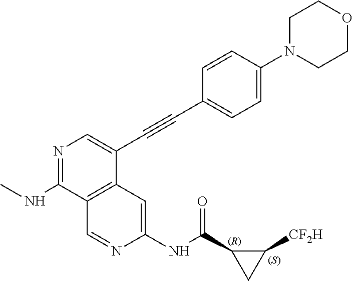 | LCMS (ESI) m/z 478.0 [M + H]⁺; chiral SFC purity: 100%; ¹H NMR (400 MHz, DMSO-d₆) δ ppm 11.30 (s, 1H), 9.37 (s, 1H), 8.62 (s, 1H), 8.32-8.17 (m, 2H), 7.43 (br d, J = 8.75 Hz, 2H), 6.98 (br d, J = 8.76 Hz, 2H), 6.28- 5.85 (m, 1H), 3.77-3.71 (m, 4H), 3.22-3.16 (m, 4H), 3.01 (d, J = 4.25 Hz, 3H), 2.45-2.39 (m, 1H), 2.00-1.84 (m, 1H), 1.42-1.28 (m, 2H). |
| | (1R,2S)-2-(difluoromethyl)-N-(8- | |
| | (methylamino)-5-((4- | |
| | morpholinophenyl)ethynyl)-2,7- | |
| | naphthyridin-3-yl)cyclopropane-1- | |
| | carboxamide | |
| | The configuration bas been arbitrarily | |
| | assigned. | |
| Example 1075 | 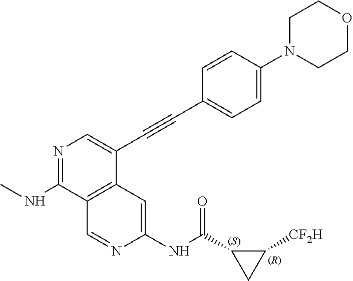 | LCMS (ESI) m/z 478.0 [M + H]⁺; chiral SFC purity: 100%; ¹H NMR (400 MHz, DMSO-d₆) δ ppm 11.30 (s, 1H), 9.37 (s, 1H), 8.62 (s, 1H), 8.32-8.17 (m, 2H), 7.43 (br d, J = 8.75 Hz, 2H), 6.98 (br d, J = 8.76 Hz, 2H), 6.28- 5.85 (m, 1H), 3.77-3.71 (m, 4H), 3.22-3.16 (m, 4H), 3.01 (d, J = 4.25 Hz, 3H), 2.45-2.39 (m, 1H), 2.00-1.84 (m, 1H), 1.42-1.28 (m, 2H). |
| | (1S,2R)-2-(difluoromethyl)-N-(8- | |
| | (methylamino)-5-((4- | |
| | morpholinophenyl)ethynyl)-2,7- | |
| | naphthyridin-3-yl)cyclopropane-1- | |
| | carboxamide | |
| | The configuration has been arbitrarily | |
| | assigned. | |
| Example 1076 | 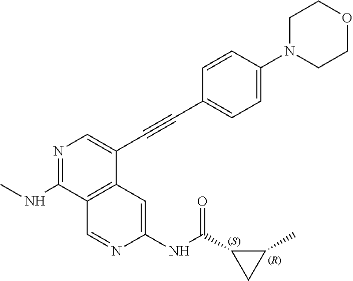 | LCMS (ESI) m/z 441.9 [M + H]⁺; chiral SFC purity: 93.8%; ¹H NMR (400 MHz, DMSO-d₆) δ ppm 10.97 (s, 1H), 9.35 (s, 1H), 8.66 (s, 1H), 8.29 (br d, J = 2.26 Hz, 1H), 8.21 (s, 1H), 7.43 (d, J = 8.78 Hz, 2H), 6.98 (d, J = 8.78 Hz, 2H), 3.80-3.68 (m, 4H), 3.24- 3.13 (m, 4H), 3.01 (d, J = 4.27 Hz, 3H), 2.15-2.05 (m, 1H), 1.35- 1.26 (m, 1H), 1.15 (d, J = 6.27 Hz, 3H), 1.00 (td, J = 8.09, 3.89 Hz, 1H), 0.82-0.91 (m, 1H). |
| | (1S,2R)-2-methyl-N-(8-(methylamino)- | |
| | 5-((4-morpholinophenyl)ethynyl)-2,7- | |
| | naphthyridin-3-yl)cyclopropane-1- | |
| | carboxamide | |
| Example 1077 | 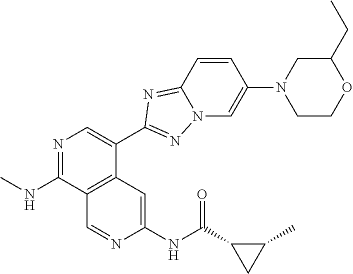 [SMILES: CCC1CN(c2ccc3nc(-c4cnc(NC)c5cnc(NC(=O)[C@H]6C[C@H]6C)cc45)nn3c2)CCO1] | LCMS (ESI) m/z 487.2 [M + H]⁺, ¹H NMR (400 MHz, DMSO-d₆) δ ppm, 10.80 (s, 1H), 9.45 (s, 1H), 9.36 (s, 1H), 8.68 (s, 1H), 8.36 (s, 2H), 7.80-7.64 (m, 2H), 3.96 (br dd, J = 11.6, 1.6 Hz, 1H), 3.72- 3.64 (m, 1H), 3.60 (br d, J = 10.4 Hz, 1H), 3.52-3.48 (m, 2H), 3.04 (d, J = 4.4 Hz, 3H), 2.72 (dt, J = 11.6, 2.8 Hz, 1H), 2.44-2.40 (m, 1H), 2.08 (dt, J = 8.0, 5.6 Hz, 1H), 1.56-1.44 (m, 2H), 1.32- 1.24 (m, 1H), 1.12 (d, J = 6.0 Hz, 3H), 1.00-0.96 (m, 1H), 0.98- 0.92 (m, 3H), 0.84-0.80 (m, 1H). |
| | (1S,2R)-N-[5-[6-(2-ethylmorpholin-4- | |
| | yl)-[1,2,4]triazolo[1,5-a]pyridin-2-yl]-8- | |
| | (methylamino)-2,7-naphthyridin-3-yl]- | |
| | 2-methyl-cyclopropanecarboxamide | |
| Example 1078 | 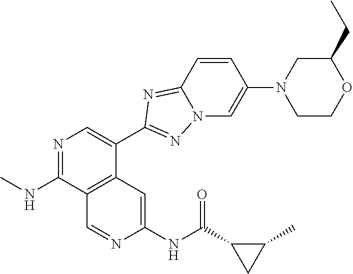 [SMILES: CC[C@@H]1CN(c2ccc3nc(-c4cnc(NC)c5cnc(NC(=O)[C@H]6C[C@H]6C)cc45)nn3c2)CCO1] | LCMS (ESI) m/z 487.4 [M + H]⁺, LCMS purity: 99.2%, HPLC purity: 100%, CHIRAL HPLC purity: 100%, ¹H NMR (400 MHz, DMSO-d₆) δ ppm 10.84 (s, 1H), 9.36 (d, J = 8.0 Hz, 2H), 8.68 (s, 1H), 8.36 (d, J = 1.3 Hz, 1H), 8.28 (br d, J = 4.5 Hz, 1H), 7.76-7.68 (m, 2H), 3.96 (br dd, J = 11.2, 1.6 Hz, 1H), 3.72-3.64 (m, 1H), 3.60 (br d, J = 11.6 Hz, 1H), 3.56-3.44 (m, 2H), 3.04 (d, J = 4.3 Hz, 3H), 2.72 (dt, J = 3.1, 11.6 Hz, 1H), 2.44-2.40 (m, 1H), 2.12-2.04 (m, 1H), 1.52 (quin, J = 7.2 Hz, 2H), 1.32-1.20 (m, 1H), 1.12 (d, J = 6.0 Hz, 3H), |
| | (1S,2R)-N-[5-[6-[(2R)-2- | 1.00-0.96 (m, 1H), 0.96-0.92 |
| | ethylmorpholin-4-yl]-[1,2,4]triazolo[1,5- | (m, 3H), 0.84-0.76 (m, 1H). |
| | a]pyridin-2-yl]-8-(methylamino)-2,7- | |
| | naphthyridin-3-yl]-2-methyl- | |
| | cyclopropanecarboxamide | |
| | Isomer obtained by chiral SFC purification | |
| | of example 1077; the configuration has | |
| | been arbitrarily assigned. | |
| Example 1079 | 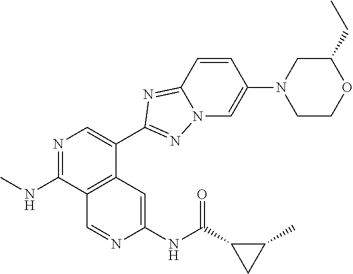 [SMILES: CC[C@H]1CN(c2ccc3nc(-c4cnc(NC)c5cnc(NC(=O)[C@H]6C[C@H]6C)cc45)nn3c2)CCO1] | LCMS (ESI) m/z 487.1 [M + H]⁺, LCMS purity: 100%, HPLC purity: 95.5%, CHIRAL HPLC purity: 98.7%, ¹H NMR (400 MHz, DMSO-d₆) δ ppm, 10.84 (s, 1H), 9.36 (d, J = 8.0 Hz, 2H), 8.68 (s, 1H), 8.36 (d, J = 1.3 Hz, 1H), 8.28 (br d, J = 4.5 Hz, 1H), 7.76-7.68 (m, 2H), 3.96 (br dd, J = 1.8, 11.3 Hz, 1H), 3.72-3.64 (m, 1H), 3.60 (br d, J = 11.6 Hz, 1H), 3.56-3.44 (m, 2H), 3.04 (d, J = 4.3 Hz, 3H), 2.72 (dt, J = 3.2, 11.6 Hz, 1H), 2.44-2.40 (m, 1H), 2.12-2.04 (m, 1H), 1.52 (quin, J = 7.2 Hz, 2H), 1.32-1.20 (m, 1H), 1.12 (d, J = 6.0 Hz, 3H), 1.00- |
| | (1S,2R)-N-[5-[6-[(2S)-2-ethylmorpholin- | 0.96 (m, 1H), 0.96-0.92 (m, 3H), |
| | 4-yl]-[1,2,4]triazolo[1,5-a]pyridin-2-yl]- | 0.84-0.76 (m, 1H). |
| | 8-(methylamino)-2,7-naphthyridin-3- | |
| | yl]-2-methyl-cyclopropanecarboxamide | |
| | Isomer obtained by chiral SFC purification | |
| | of example 1077; the configuration has | |
| | been arbitrarily assigned. | |
| Example 1080 | 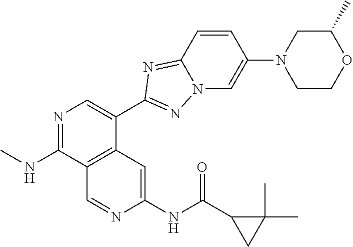 | LCMS (ESI) m/z 487.3 [M + H]⁺, ¹H NMR (400 MHz, DMSO-d₆) δ ppm, 10.76 (s, 1H), 9.38 (s, 1H), 9.35 (s, 1H), 8.69 (s, 1H), 8.34 (s, 1H), 8.24 (br s, 1H), 7.64-7.79 (m, 2H), 3.94 (br d, J = 9.6 Hz, 1H), 3.67-3.74 (m, 2H), 3.58- 3.67 (m, 2H), 3.04 (d, J = 4.4 Hz, 3H), 2.65-2.76 (m, 1H), 2.40 (t, J = 11.2 Hz, 1H), 1.93 (d, J = 5.6 Hz, 1H), 1.11-1.20 (m, 9H), 1.02 (t, J = 4.4 Hz, 1H), 0.82 (dd, J = 8.0, 4.0 Hz, 1H). |
| | 2,2-dimethyl-N-(8-(methylamino)-5-(6- | |
| | ((S)-2-methylmorpholino)- | |
| | [1,2,4]triazolo[1,5-a]pyridin-2-yl)-2,7- | |
| | naphthyridin-3-yl)cyclopropane-1- | |
| | carboxamide | |
| Example 1081 | 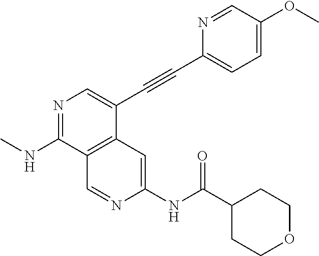 | LCMS (ESI) m/z 418.2 [M + H]⁺, ¹H NMR (400 MHz, DMSO-d₆) δ ppm, 10.92-11.09 (m, 1H), 9.58 (br s, 1H), 8.73 (br s, 1H), 8.36 (br s, 1H), 8.23 (br s, 1H), 7.69 (br d, J = 7.6 Hz, 1H), 7.52 (br d, J = 7.6 Hz, 1H), 3.90-3.95 (m, 2H), 3.89 (br s, 3H), 3.23-3.38 (m, 2H), 3.09 (br s, 3H), 2.84 (br s, 1H), 1.60-1.81 (m, 4H). |
| | N-[5-[2-(5-methoxy-2-pyridyl)ethynyl]- | |
| | 8-(methylamino)-2,7-naphthyridin-3- | |
| | yl]tetrahydropyran-4-carboxamide | |
| Example 1082 | 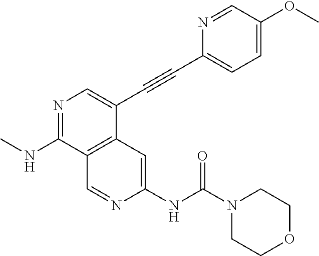 | LCMS (ESI) m/z 419.1 [M + H]⁺, ¹H NMR (400 MHz, DMSO-d₆) δ ppm 9.52 (s, 1H), 9.33 (s, 1H), 8.34 (s, 1H), 8.32 (d, J = 3.2 Hz, 1H), 8.27 (s, 1H), 7.61 (d, J = 8.8 Hz, 1H), 7.47 (dd, J = 8.8, 3.2 Hz, 1H), 3.88 (s, 3H), 3.57-3.64 (m, 4H), 3.47-3.54 (m, 4H), 3.01 (d, J = 4.0 Hz, 3H). |
| | N-[5-[2-(5-methoxy-2-pyridyl)ethynyl]- | |
| | 8-(methylamino)-2,7-naphthyridin-3- | |
| | yl]morpholine-4-carboxamide | |
| Example 1083 | 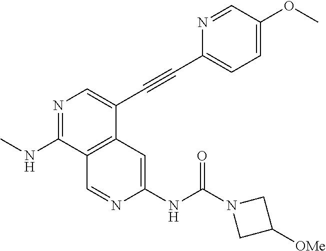 | LCMS (ESI) m/z 419.5 [M + H]⁺, ¹H NMR (400 MHz, CD₃OD) δ ppm, 9.42 (s, 1H), 8.68 (s, 1H), 8.54 (br s, 1H), 8.07-8.20 (m, 2H), 7.95-8.04 (m, 1H), 4.35 (br d, J = 8.4 Hz, 2H), 4.30 (br d, J = 5.6 Hz, 1H), 4.06 (s, 3H), 4.00 (br d, J = 6.4 Hz, 2H), 3.34 (s, 3H), 3.28 (s, 3H). |
| | 3-methoxy-N-[5-[2-(5-methoxy-2- | |
| | pyridyl)ethynyl]-8-(methylamino)-2,7- | |
| | naphthyridin-3-yl]azetidine-1- | |
| | carboxamide | |
| Example 1084 | 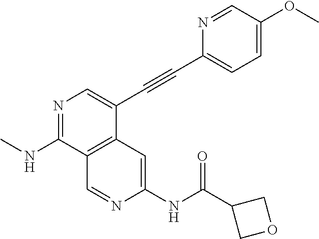 | LCMS (ESI) m/z 390.0 [M + H]⁺, ¹H NMR (400 MHz, DMSO-d₆) δ ppm 10.04 (br s, 1H), 9.31 (br s, 1H), 8.36 (s, 1H), 8.33 (d, J = 2.8 Hz, 1H), 7.69 (d, J = 8.4 Hz, 1H), 7.46 (dd, J = 8.4, 2.8 Hz, 1H), 7.34 (br s, 1H), 5.09 (br s, 1H), 4.70-4.81 (m, 1H), 4.53-4.68 (m, 1H), 3.89 (s, 3H), 3.81 (br d, J = 11.6 Hz, 1H), 3.64 (br d, J = 7.6 Hz, 1H), 3.04 (d, J = 3.6 Hz, 3H), 2.97 (br s, 1H). |
| | N-[5-[2-(5-methoxy-2-pyridyl)ethynyl]- | |
| | 8-(methylamino)-2,7-naphthyridin-3- | |
| | yl]oxetane-3-carboxamide | |
| Example 1085 | 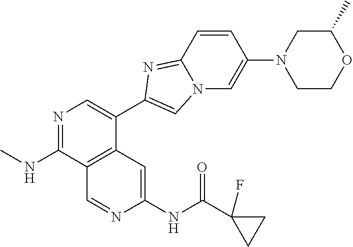 | LCMS (ESI) m/z 476.0 [M + H]⁺, ¹H NMR (400 MHz, DMSO-d₆) δ 10.30 (s, 1H), 9.44 (s, 1H), 8.94 (s, 1H), 8.33 (s, 1H), 8.15-8.07 (m, 1H), 8.04 (s, 1H), 8.00 (s, 1H), 7.49-7.47 (d, J = 10.0 Hz, 1H), 7.28-7.26 (d, J = 10.0 Hz, 1H), 3.96-3.88 (m, 1H), 3.75-3.65 (m, 2H), 3.47 (br d, J = 11.6 Hz, 1H), 3.39 (br d, J = 11.2 Hz, 1H), 3.04-3.01 (m, 3H), 2.62 (br d, J = 3.3 Hz, 1H), 2.44 (br s, 1H), 2.34- 2.27 (m, 2H), 1.49-1.41 (m, 2H), 1.16 (d, J = 6.4 Hz, 3H). |
| | 1-fluoro-N-[8-(methylamino)-5-[6-[(2S)- | |
| | 2-methylmorpholin-4-yl]imidazo[1,2- | |
| | a]pyridin-2-yl]-2,7-naphthyridin-3- | |
| | yl]cyclopropanecarboxamide | |

### Example 1086

### Synthesis of N-[2-[6-[(6-methoxy-2-pyridyl)amino]-1-(methylamino)-2,7-naphthyridin-4-yl]-1,3-benzoxazol-5-yl]-N-methyl-acetamide

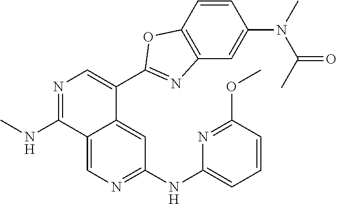 [SMILES: CNc1ncc(-c2nc3cc(N(C)C(C)=O)ccc3o2)c2cc(Nc3cccc(OC)n3)ncc12]

Synthesis of N-[2-[6-[(6-methoxy-2-pyridyl)amino]-1-(methylamino)-2,7-naphthyridin-4-yl]-1,3-benzoxazol-5-yl]-N-methyl-acetamide was synthesized using N₆-(6-methoxypyridin-2-yl)-N₁-methyl-4-(5-(methylamino)benzo[d]oxazol-2-yl)-2,7-naphthyridine-1,6-diamine and acetic acid as the starting material with TCFH, NMI condition. LCMS (ESI) m/z 470.1 [M+H]⁺. ¹H NMR (400 MHz, DMSO-d₆) δ ppm 10.03 (s, 1H) 9.38 (d, J=4.5 Hz, 2H), 8.86 (s, 1H), 8.59 (br d, J=4.5 Hz, 1H), 7.79 (br d, J=8.4 Hz, 1H), 7.70 (s, 1H), 7.62 (t, J=7.9 Hz, 1H), 7.35 (br d, J=8.6 Hz, 1H), 6.90 (d, J=7.9 Hz, 1H), 6.35 (d, J=7.9 Hz, 1H), 3.82 (s, 3H), 3.22 (s, 3H), 3.08 (d, J=4.5 Hz, 3H), 1.80 (s, 3H).

### Example 1087

### Synthesis of 3-[[5-[2-(5-methoxy-2-pyridyl)ethynyl]-8-(methylamino)-2,7-naphthyridin-3-yl]amino]-1-(2-pyridyl)pyridin-2-one

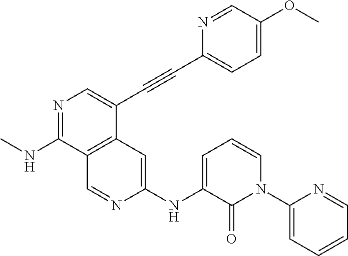 [SMILES: CNc1ncc(C#Cc2ccc(OC)cn2)c2cc(Nc3cccn(-c4ccccn4)c3=O)ncc12]

Step 1. Synthesis to 3-bromo-1-(2-pyridyl)pyridin-2-on: A mixture of 3-bromopyridin-2-ol (1 g, 5.8 mmol, 1 eq), 2-bromopyridine (1.82 g, 11.5 mmol, 2 eq), DMEDA (50.7 mg, 0.57 mmol, 0.1 eq), CuI (54.7 mg, 0.29 mmol, 0.05 eq) and K₃PO₄ (2.44 g, 11.5 mmol, 2 eq) in toluene (1.0 mL) was degassed and purged with N₂ for 3 times, and then the mixture was stirred at 120° C. for 16 hrs under N₂ atmosphere. The reaction mixture was filtered and concentrated under reduced pressure to give a residue. The residue was purified by column chromatography (ISCO®; 20 g SepaFlash® Silica Flash Column, petroleum ether/EtOAc with EtOAc from 0-60%, 40 mL/min, 254 nm) to afford 3-bromo-1-(2-pyridyl)pyridin-2-one (1 g, 69.3% yields) as a white solid. LCMS (ESI) m/z 252.7 [M+H]⁺.

### Step 2. Synthesis of 3-[[5-[2-(5-methoxy-2-pyridyl)ethynyl]-8-(methylamino)-2,7-naphthyridin-3-yl]amino]-1-(2-pyridyl)pyridin-2-one

A mixture of 4-[2-(5-methoxy-2-pyridyl)ethynyl]-N1-methyl-2,7-naphthyridine-1,6-diamine (50 mg, 0.16 mmol, 1 eq), 3-bromo-1-(2-pyridyl)pyridin-2-one (49.3 mg, 0.2 mmol, 1.2 eq), Cs₂CO₃ (160 mg, 0.49 mmol, 3 eq), Pd₂(dba)₃ (30 mg, 0.033 mmol, 0.2 eq) and DavePhos (25.8 mg, 0.066 mol, 0.4 eq) in dioxane (2 mL) was degassed and purged with N₂ for 3 times, and then the mixture was stirred at 120° C. for 16 hours under N₂ atmosphere. The reaction mixture was concentrated under reduced pressure to give a residue. The residue was purified by column chromatography (ISCO®; 4 g SepaFlash® Silica Flash Column, DCM/MeOH with MeOH from 0-2.9%, 30 mL/min, 254 nm) to afford 3-[[5-[2-(5-methoxy-2-pyridyl)ethynyl]-8-(methylamino)-2,7-naphthyridin-3-yl]amino]-1-(2-pyridyl)pyridin-2-one (43 mg, 51.9% yields) as a yellow solid. LCMS (ESI) m/z 476.2 [M+H]⁺, ¹H NMR (400 MHz, DMSO-d₆) δ ppm 9.32 (s, 1H), 9.12 (s, 1H), 8.64 (d, J=3.6 Hz, 1H), 8.48 (dd, J==7.6, 1.6 Hz, 1H), 8.31 (d, J=° 3.2 Hz, 2H), 8.20 (s, 1H), 8.03 (td, J=7.6, 1.6 Hz, 1H), 7.84 (d, J=8.0 Hz, 1H), 7.73 (d, J=8.4 Hz, 1H), 7.63 (s, 1H), 7.52-7.55 (m, 1H), 7.50 (dd, J==7.2, 1.6 Hz, 1H), 7.46 (dd, J=8.8, 2.8 Hz, 1H), 6.47 (t, J=7.2 Hz, 1H), 3.88 (s, 3H), 3.03 (d, J=4.4 Hz, 3H).

### Example 1088: Synthesis of 2-[2-[[8-(methylamino)-5-(6-morpholino-[1,2,4]triazolo[1,5-a]pyridin-2-yl)-2,7-naphthyridin-3-yl]amino]-4-pyridyl]propan-2-ol

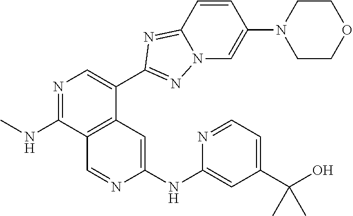 [SMILES: CNc1ncc(-c2nc3ccc(N4CCOCC4)cn3n2)c2cc(Nc3cc(C(C)(C)O)ccn3)ncc12]

Step 1. Synthesis of methyl 2-[[8-(methylamino)-5-(6-morpholino-[1,2,4]triazolo[1,5-a]1403yridine-2-yl)-2,7-naphthyridin-3-yl]amino]pyridine-4-carboxylate was synthesized using a similar procedure that was previously described in Example 1087, Step 2, by using N¹-methyl-4-(6-morpholino-[1,2,4]triazolo[1,5-a]pyridin-2-yl)-2,7-naphthyridine-1,6-diamine and methyl 2-bromoisonicotinate as the starting material. LCMS (ESI) m/z 512.2 [M+H]⁺.

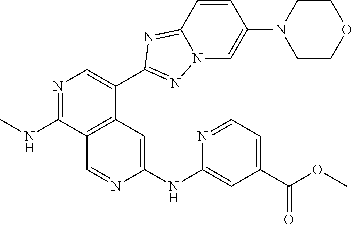 [SMILES: CNc1ncc(-c2nc3ccc(N4CCOCC4)cn3n2)c2cc(Nc3cc(C(=O)OC)ccn3)ncc12]

Step 2. Synthesis of 2-[2-[[8-(methylamino)-5-(6-morpholino-[1,2,4]triazolo[1,5-a]pyridin-2-yl)-2,7-naphthyridin-3-yl]amino]-4-pyridyl]propan-2-del: To a solution of methyl 2-[[8-(methylamino)-5-(6-morpholino-[1,2,4]triazolo[1, 5-a]pyridin-2-yl)-2,7-naphthyridin-3-yl]amino]pyridine-4-carboxylate (50 mg, 0.1 mmol, 1 eq) in THF (2 mL) was added McMgBr (3 M, 0.1 mL, 30 eq) at 0° C., then the mixture was stirred at 20° C. for 1 hr. The reaction mixture was diluted with NH₄Cl aqueous solution 5 mL and extracted with EtOAc (20 mL×3), dried over Na₇SO₄, concentrated under reduced pressure to give a residue. The residue was purified by prep-TLC (DCM:MeOH=5:1) to afford 2-[2-[[8-(methylamino)-5-(6-morpholino-[1,2,4]triazolo[1,5-a]pyridin-2-yl)-2,7-naphthyridin-3-yl]amino]-4-pyridyl]propan-2-ol (33 mg, 63.4% yields) as a yellow solid. LCMS (ESI) m/z 512.4 [M+H]⁺. ¹H NMR (400 MHz, DMSO-d₆) δ ppm 10.13 (s, 1H), 9.49 (br s, 1H), 9.39 (s, 1H), 8.65 (br s, 1H), 8.35 (s, 1H), 8.23 (d, J=5.6 Hz, 1H), 7.74-7.79 (m, 1H), 7.68-7.74 (m, 1H), 7.58 (s, 1H), 7.05 (br d, J=4.4 Hz, 1H), 5.25 (br s, 1H), 3.78-3.83 (m, 4H), 3.15-3.20 (m, 4H), 3.07 (d, J=4.4 Hz, 3H), 1.43 (s, 6H).

### Example 1089: Synthesis of 2-[3-fluoro-2-[[8-(methylamino)-5-(6-morpholino-[1,2,4]triazolo[1,5-a]pyridin-2-yl)-2,7-naphthyridin-3-yl]amino]-4-pyridyl]propan-2-ol

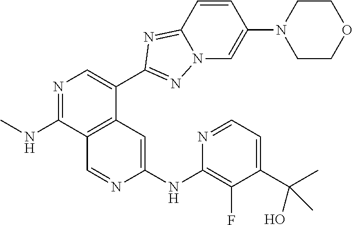 [SMILES: CNc1ncc(-c2nc3ccc(N4CCOCC4)cn3n2)c2cc(Nc3nccc(C(C)(C)O)c3F)ncc12]

Step 1. Methyl 3-fluoro-2-[[8-(methylamino)-5-(6-morpholino-[1,2,4]triazolo[1,5-a]pyridin-2-yl)-2,7-naphthyridin-3-yl]amino]pyridine-4-carboxylate was synthesized using a similar procedure that was previously described in Example 1089, Step 2 by using N¹-methyl-4-(6-morpholino-[1,2,4]triazolo[1,5-a]pyridin-2-yl)-2,7-naphthyridine-1,6-diamine and methyl 2-bromo-3-fluoroisonicotinate as the starting material. LCMS (ESI) m/z 530.3 [M+H]⁺.

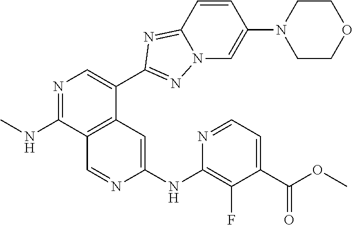 [SMILES: CNc1ncc(-c2nc3ccc(N4CCOCC4)cn3n2)c2cc(Nc3nccc(C(=O)OC)c3F)ncc12]

Step 2. Synthesis of 2-[3-fluoro-2-[[8-(methylamino)-5-(6-morpholino-[1,2,4]triazolo[1,5-a]pyridin-2-yl)-2,7-naphthyridin-3-yl]amino]-4-pyridyl]propan-2-ol: To a solution of methyl 3-fluoro-2-[[8-(methylamino)-5-(6-morpholino-[1,2,4]triazolo[1,5-a]pyridin-2-yl)-2,7-naphthyridin-3-yl]amino]pyridine-4-carboxylate (50 mg, 48.16 μmol, 1 eq) in THF (2 mL) was added MeMgBr (3 M, 321.04 μL, 20 eq) at 0° C. The mixture was stirred at 20° C. for 1 hr. The reaction mixture was diluted with NH₄Cl aqueous solution (5 mL) and extracted with EtOAc (20 mL×3), dried over Na₂SO₄, and concentrated under reduced pressure. The residue was purified by prep-TLC (SiO2, DCM:MeOH=10:1) to afford 2-[3-fluoro-2-[[8-(methylamino)-5-(6-morpholino-[1,2,4]triazolo[1,5-a]pyridin-2-yl)-2,7-naphthyridin-3-yl]amino]-4-pyridyl]propan-2-ol (11 mg, 42.9% yields) as a yellow solid. LCMS (ESI) m/z 530.3 [M+H]⁺, ¹H NMR (400 MHz, DMSO-d₆) δ ppm 9.51-9.55 (m, 1H), 9.32 (s, 1H), 9.16 (s, 1H), 8.76 (s, 1H), 8.32 (d, J=1.6 Hz, 1H), 8.1.7 (br d, J=5.2 Hz, 1H), 8.13 (d, J=5.2 Hz, 1H), 7.71-7.77 (m, 1H), 7.65-7.70 (m, 7.23 (t, J=5.2 Hz, 1H), 5.49 (s, 1H), 3.77-3.82 (m, 4H), 3.13-3.19 (m, 4H), 3.02-3.06 (m, 3H), 1.51. (s, 6H).

### Example 1.090: Synthesis of N₁-methyl-4-(6-morpholino-[1,2,4]triazolo[1,5-a]pyridin-2-yl)-N6-[5-(trifluoromethyl)-2-pyridyl]-2,7-naphthyridine-1,6-diamine

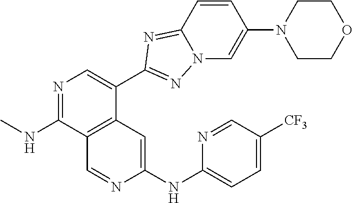 [SMILES: CNc1ncc(-c2nc3ccc(N4CCOCC4)cn3n2)c2cc(Nc3ccc(C(F)(F)F)cn3)ncc12]

Synthesis of Example 1090: N1-methyl-4-(6-morpholino-[1,2,4]triazolo[1,5-a]pyridin-2-yl)-N6-[5-(trifluoromethyl)-2-pyridyl]-2,7-naphthyridine-1,6-diamine was synthesized using a similar procedure that was previously described in Example 1087 by using N1-methyl-4-(6-morpholino-[1,2,4]triazolo[1,5-a]pyridin-2-yl)-2,7-naphthyridine-1,6-diamine and 2-bromo-5-(trifluoromethyl)pyridine as the starting material. LCMS (ESI) m/z 522.2 [M+H]⁺, ¹H NMR (400 MHz, DMSO-d₆) δ ppm 10.50 (s, 1H), 9.62 (s, 1H), 9.39 (s, 1H), 8.77 (s, 1H), 8.65 (s, 1H), 8.38 (s, 1H), 8.24 (br s, 1H), 8.03 (br d, J=9.2 Hz, 1H), 7.74-7.80 (m, 1H), 7.63-7.72 (m, 2H), 3.76-3.83 (m, 4H), 3.18 (br d, J=4.4 Hz, 4H), 3.05 (d, J=4.4 Hz, 3H).

### Example 1091: Synthesis of -(2-(6-(cyclopropanecarboxamido)-1-(methylamino)-2,7-naphthyridin-4-yl)benzo[d]oxazol-5-yl)cyclopropanecarboxamide

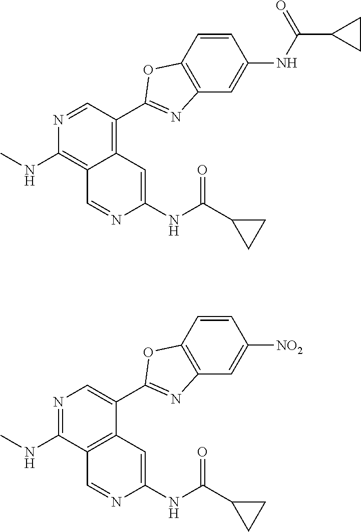 [SMILES: CNc1ncc(-c2nc3cc(NC(=O)C4CC4)ccc3o2)c2cc(NC(=O)C3CC3)ncc12 ; CNc1ncc(-c2nc3cc([N+](=O)[O-])ccc3o2)c2cc(NC(=O)C3CC3)ncc12]

Step 1: Synthesis of N-(8-(methylamino)-5-(5-nitrobenzo[d]oxazol-2-yl)-2,7-naphthyridin-3-yl)cyclopropanecarboxamide was synthesized using a similar procedure that was previously described in Example 916 by using N-(5-bromo-8-(methylamino)-2,7-naphthyridin-3-yl)cyclopropanecarboxamide and 5-nitrobenzo[d]oxazole as the starting material. LCMS (ESI) m/z 405.1 [M+H]⁺.

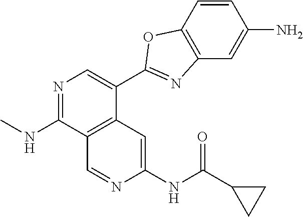 [SMILES: CNc1ncc(-c2nc3cc(N)ccc3o2)c2cc(NC(=O)C3CC3)ncc12]

### Step 2: N-(5-(5-aminobenzo[d]oxazol-2-yl)-8-(methylamino)-2,7-naphthyridin-3-yl)cyclopropanecarboxamide

To a solution of N-[8-(methylamino)-5-(5-nitro-1,3-benzoxazol-2-yl)-2,7-naphthyridin-3-yl]cyclopropanecarboxamide (130 mg, 0.32 mmol, 1 eq) in EtOH (4 mL) and H₂O (1 mL) were added Fe (89.8 mg, 1.61 mmol, 5 eq) and NH C1 (85.9 mg, 1.61 mmol, 5 eq). The mixture was stirred at 80° C. for 1 hr. Then the reaction mixture was filtered and concentrated under reduced pressure. The residue was purified by flash silica gel chromatography (ISCO®; 4 g SepaFlash® Silica Flash Column, Eluent of 0-10% Dichloromethane Methanol gradient @ 30 mL/min) to give N-(5-(5-aminobenzo[d]oxazol-2-yl)-8-(methylamino)-2,7-naphthyridin-3-yl)cyclopropanecarboxamide (60 mg, 43.4% yield) as a yellow solid. LCMS (ESI) m/z 375.1 [M+H]⁺.

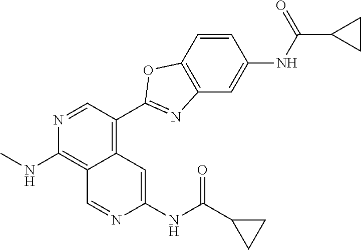 [SMILES: CNc1ncc(-c2nc3cc(NC(=O)C4CC4)ccc3o2)c2cc(NC(=O)C3CC3)ncc12]

### Step 3. Synthesis of N-(2-(6-(cyclopropanecarboxamido)-1-(methylamino)-2,7-naphthyridin-4-yl)-benzo[d]oxazol-5-yl)cyclopropanecarboxamide

To a solution of N-[₅-(5-amino-1,3-benzoxazol-2-yl)-8-(methylamino)-2,7-naphthyridin-3-yl]cyclopropanecarboxamide (50 mg, 0.13 mmol, 1 eq) and cyclopropanecarboxylic acid (17.3 mg, 0.2 mmol, 1.5 eq) in DCM (1 mL) was added pyridine (106 mg, 1.34 mmol, 10 eq) and POCl₃ (102 mg, 0.67 mmol, 5 eq) at 0° C. Then the mixture was stirred at 25° C. for 2 hrs. The residue was diluted with H₂O (2 mL) and concentrated under reduced pressure. The residue was purified by prep-HPLC (column: Phenomenex Synergi C18 100×30 mm×4 μm; mobile phase: [water (HCl)-ACN]; gradient:15%-45% B over 8 min) to give N-(2-(6-(cyclopropanecarboxamido)-1-(methylamino)-2,7-naphthyridin-4-yl)benzo[d]oxazol-5-yl)cyclopropanecarboxamide (24.63 mg, 41.2% yield) as a yellow solid. LCMS (ESI) m/z 443.1 [M+H]⁺, ¹H NMR (400 MHz, DMSO-d₆) δ ppm 11.28 (Iv s, 1H), 10.43 (s, 1H), 9.83 (s, 1H), 9.61 (s, 1H), 8.65 (s, 1H), 8.22 (s, 1H), 7.70 (d, J=8.8 Hz, 1H), 7.51 (dd, J=8.8, 1.6 Hz, 1H), 3.17 (br d, J=4.0 Hz, 3H), 2.09-2.14 (m, 1H), 1.78-1.84 (m, 1H), 0.88-0.97 (m, 4H), 0.81-0.86 (m, 4H).

### Example 1092: Synthesis of N-(8-(methylamino)-5-(5-propionamidobenzo[d]oxazol-2-yl)-2,7-naphthyridin-3-yl)cyclopropanecarboxamide

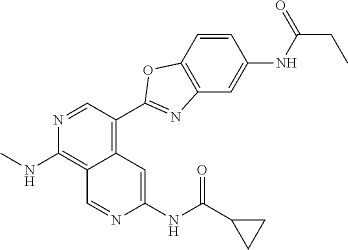 [SMILES: CCC(=O)Nc1ccc2oc(-c3cnc(NC)c4cnc(NC(=O)C5CC5)cc34)nc2c1]

Synthesis of N-(8-(methylamino)-5-(5-propionamidobenzo[d]oxazol-2-yl)-2,7-naphthyridin-3-yl)cyclopropanecarboxamide was synthesized using a similar procedure that was previously described in Example 1091 by using N-[5-(5-amino-1,3-benzoxazol-2-yl)-8-(methylamino)-2,7-naphthyridin-3-yl]cyclopropanecarboxamide and propionic acid as the starting material. LCMS (ESI) m/z 431.1 [M+H]⁺; ¹H NMR (400 MHz, DMSO-d₆) δ ppm 11.21 (br s, 1H), 10.05 (s, 1H), 9.81 (s, 1H), 9.54 (s, 1H), 8.72 (s, 1H), 8.21 (d, J=1.6 Hz, 1H), 7.68 (d, J=8.8 Hz, 1H), 7.49 (dd, J=8.8, 1.6 Hz, 1H), 3.15 (br d, J=4.0 Hz, 3H), 2.37 (d, J=7.6 Hz, 2H), 2.11 (dt, J=8.0, 3.6 Hz, 1H), 1.12 (t, J=8.0 Hz, 3H), 0.87-0.95 (m, 4H).

### Example 1093: Synthesis of N-(5-(5-(2-cyclopropylacetamido)benzo[d]oxazol-2-yl)-8-(methylamino)-2,7-naphthyridin-3-yl)cyclopropanecarboxamide

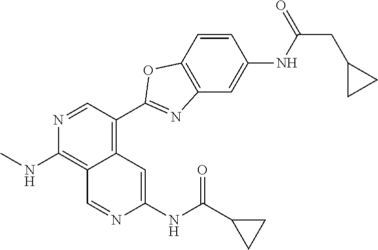 [SMILES: CNc1ncc(-c2nc3cc(NC(=O)CC4CC4)ccc3o2)c2cc(NC(=O)C3CC3)ncc12]

Synthesis of N-(5-(5-(2-cyclopropylacetamido)benzo[d]oxazol-2-yl)-8-(methylamino)-2,7-naphthyridin-3-yl)cyclopropanecarboxamide was synthesized using a similar procedure that was previously described in example 1091 by using N-[5-(5-amino-1,3-benzoxazol-2-yl)-8-(methylamino)-2,7-naphthyridin-3-yl]cyclopropanecarboxamide and 2-cyclopropylacetic acid as the starting material. LCMS (ESI) m/z 457.1, [M+H]⁺; NMR (400 MHz, DMSO-d₆) δ ppm 11.33 (s, 1H), 10.08 (s, 1H), 9.86 (s, 1H), 9.66 (s, 1H), 8.60 (s, 1H), 8.25 (d, J=1.6 Hz, 1H), 7.71 (d, J=8.0 Hz, 1H), 7.52 (dd, J=8.8, 1.6 Hz, 1H), 3.19 (br d, J=4.0 Hz, 3H), 2.26 (d, J=8.0 Hz, 2H), 2.11 (br d, J=4.8 Hz, 1H), 1.06-1.14 (m, 1H), 0.87-0.96 (m, 4H), 0.50 (dd, J=8.0, 1.6 Hz, 2H), 0.23 (d, J=4.8 Hz, 2H).

### Example 1094: Synthesis of N-(8-(methylamino)-5-(6-morpholinobenzo[d]thiazol-2-yl)-2,7-naphthyridin-3-yl)cyclopropanecarboxamide

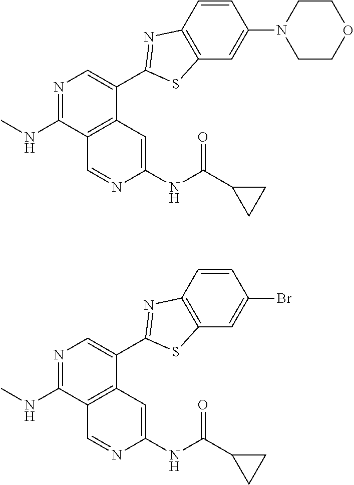 [SMILES: CNc1ncc(-c2nc3ccc(N4CCOCC4)cc3s2)c2cc(NC(=O)C3CC3)ncc12 ; CNc1ncc(-c2nc3ccc(Br)cc3s2)c2cc(NC(=O)C3CC3)ncc12]

Step 1. Synthesis of N-(5-(6-bromobenzo[d]thiazol-2-yl)-8-(methylamino)-2,7-naphthyridin-3-yl)cyclopropanecarboxamide. A mixture of 6-bromo-2-chloro-1,3-benzothiazole (300 mg, 1.21 mmol), N-[8-(methylamino)-5-(4,4,5,5-tetramethyl-1,3,2-dioxaborinan-2-yl)-2,7-naphthyridin-3-yl]cyclopropanecarboxamide (500 mg, 1.36 mmol), Pd(dppf)Cl₂ (90 mg, 0.123 mmol) and K₂CO₃ (420 mg, 3.04 mmol) were taken up into a microwave tube in dioxane (6 mL)/H₂O (0.6 mL) and the sealed tube was heated at 100° C. for 3 h under microwave reactor. The residue was diluted with H₂O (30 mL) and extracted with DCM (50 mL×3). The combined organic layers were washed with brine (30 mL), dried over anhydrous Na₂SO₄, filtered and concentrated under reduced pressure. The residue was purified by flash chromatography (ISCO®; 12 g SepaFlash® Silica Flash Column, Petroleum ether/EtOAc with EtOAc from 0-100%, flow rate=30 mL/min, 254 nm), to provide N-[5-(6-bromo-1,3-benzothiazol-2-yl)-8-(methylamino)-2,7-naphthyridin-3-yl]cyclopropanecarboxamide (350 mg, 63.8% yield) as a yellow solid. LCMS (ESI) m/z 455.8 [M+H]⁺.

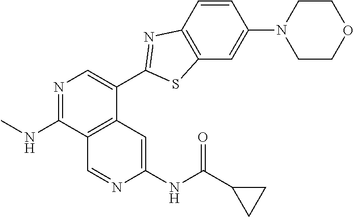 [SMILES: CNc1ncc(-c2nc3ccc(N4CCOCC4)cc3s2)c2cc(NC(=O)C3CC3)ncc12]

Step 2. To a solution of N-[5-(6-bromo-1,3-benzothiazol-2-yl)-8-(methylamino)-2,7-naphthyridin-3-yl]cyclopropanecarboxamide (330 mg, 0.726 mmol), morpholine (250 mg, 2.87 mmol), tBuONa (180 mg, 1.87 mmol) in dioxane (30 mL) was added RuPhos-Pd-G₃ (61 mg, 0.0729 mmol) and RuPhos (70 mg, 0.1.50 mmol). The mixture was stirred at 100° C. for 12 hrs. The reaction mixture was quenched by addition water (50 mL) and extracted with EtOAc (30 mL×3). The combined organic layers were washed with brine (60 mL×2), dried over anhydrous Na₂SO₄, filtered, and concentrated under reduced pressure. The residue was purified by flash silica gel chromatography (ISCO®; 20 g SepaFlash® Silica Flash Column, Petroleum ether/EtOAc from 0-80%, flow rate: 60 mL/min, 254 nm) to afford N-[8-(methylamino)-5-(6-morpholino-1,3-benzothiazol-2-yl)-2,7-naphthyridin-3-yl]cyclopropanecarboxamide (10 mg, 2.9% yield) as a yellow solid. LCMS (ESI) m/z 461.0 [M+H]⁺; ¹H NMR (400 MHz, DMSO-d₆) ppm 11.01 (s, 1H), 9.52 (s, 1H), 9.42 (s, 1H), 8.52 (s, 1H), 8.46 (br d, J=4.3 Hz, 1H), 7.82 (d, 0.1=9.0 Hz, 1H), 7.59 (d, J=2.3 Hz, 1H), 7.23 (m, 1H), 3.75-3.82 (m, 4H), 3.19-3.22 (m, 4H), 3.06 (d, J=4.3 Hz, 3H), 2.01-2.13 (m, 1H), 0.78-0.88 (m, 4H).

### Example 1095

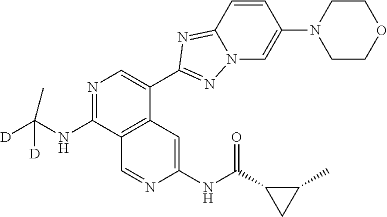 [SMILES: [2H]C([2H])(C)Nc1ncc(-c2nc3ccc(N4CCOCC4)cn3n2)c2cc(NC(=O)[C@H]3C[C@H]3C)ncc12]

### Step 1: Synthesis of 6-chloro-N-(ethyl-1,1-d₂)-2,7-naphthyridin-1-amine

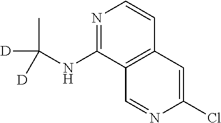 [SMILES: [2H]C([2H])(C)Nc1nccc2cc(Cl)ncc12]

To a solution of lithium tetradeuterioalumanuide (342.48 mg, 9.02 mmol, 2 eq) in THF (20 mL) at 0° C., N-(6-chloro-2,7-naphthyridin-1-yl)acetamide (1 g, 4.51 mmol, 1 eq) was slowly added. The reaction mixture was stirred at 0° C. for 1 hr. The reaction was quenched by H₂O (0.1.4 mL) at 0° C. Then 15% NaOH: aq. (0.14 mL) and H₂O (0.42 mL) was followed. Then the reaction mixture was filtered by celite, and the filtration was concentrated under reduced pressure. The residue was purified by flash silica gel chromatography (ISCO®; 12 g SepaFlash® Silica Flash Column, Eluent of 0-50% Ethyl acetate/Petroleum ether gradient @ 25 mL/min) to afford 6-chloro-N-(ethyl-1,1-d₂)-2,7-naphthyridin-1-amine (236 mg, 24.95% yield) as a white solid. LCMS (ESI) m/z 209.8 [M+H]⁺.

### Step 2: Synthesis of (1S,2R)—N-(8-((ethyl-1,1-d₂)amino)-2,7-naphthyridin-3-yl)-2-methylcyclopropane-1-carboxamide

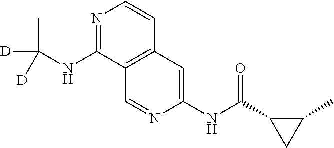 [SMILES: [2H]C([2H])(C)Nc1nccc2cc(NC(=O)[C@H]3C[C@H]3C)ncc12]

A mixture of 6-chloro-N-(ethyl-1,1-d₂)-2,7-napthyridine-1-amine (200 mg, 0.95 mmol, 1 eq), (1S,2R)-2-methylcyclopropane-1-carboxamide (94.56 mg, 0.95 mmol, 1 eq), BrettPhos (102.40 mg, 0.19 mmol, 0.2 eq), BrettPhos-Pd-G₃ (172.94 mg, 0.19 mmol, 0.2 eq) and Cs₂CO₃ (932.37 mg, 2.86 mmol, 3 eq) in dioxane (8 mL) was degassed and purged with N₂ for 3 times, and then the mixture was stirred at 100° C. for 16 hrs under N₂ atmosphere. The reaction mixture was filtered and the filtration was concentrated under reduced pressure. The residue was purified by flash silica gel chromatography (ISCO®; 12 g SepaFlash® Silica Flash Column, Eluent of 0-80% Ethyl acetate/Petroleum ether gradient @ 30 mL/min) to afford (1S,2R)—N-(8-((ethyl-1,1-d₂)amino)-2,7-naphthyridin-3-yl)-2-methyl cyclopropane-1-carboxamide (157 mg, 60.44% yield) as a pale yellow solid. LCMS (ESI) m/z 272.9 [M+H]⁺.

### Step 3: Synthesis of (1S,2R)—N-(5-bromo-8-((ethyl-1,1-d₂)amino)-2,7-naphthyridin-3-yl)-2-methylcyclopropane-1-carboxamide

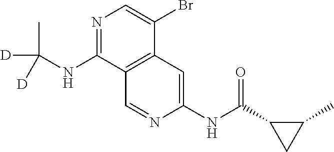 [SMILES: [2H]C([2H])(C)Nc1ncc(Br)c2cc(NC(=O)[C@H]3C[C@H]3C)ncc12]

To a solution of (1S,2R)—N-(8-((ethyl-1,1-d₂)amino)-2,7-naphthyridin-3-yl)-2-methylcyclopropane-1-carboxamide (157 mg, 0.57 mmol, 1 eq) in DMF (3 mL), NBS (112.87 mg, 0.63 mmol, 1.1 eq) was added. Then the reaction mixture was stirred at 25° C. for 1 hr. The reaction mixture was partitioned between EtOAc (5 mL) and H₂O (5 mL). Then the water phase was extracted by EtOAc (5 mL×3). The organic phases were combined and washed with H₂O (10 mL) and brine (10 mL). Then the residue was concentrated under reduced pressure. The residue was purified by flash silica gel chromatography (ISCO®; 12 g SepaFlash® Silica Flash Column, Eluent of 0-50% Ethyl acetate/Petroleum ether gradient @ 30 mL/min) to afford (1S,2R)—N-(5-bromo-8-((ethyl-1,1-d₂)amino)-2,7-naphthyridin-3-yl)-2-methylcyclopropane-1-carboxamide (123 mg, 60.7% yield) as a yellow solid. LCMS (ESI) m/z 352.9 [M+H]⁺.

### Step 4: Synthesis of (1S,2R)—N-(8-((ethyl-1,1-d₂)amino)-5-(4,4,5,5-tetramethyl-1,3,2-dioxaborolan-2-yl)-2,7-naphthyridin-3-yl)-2-methylcyclopropane-1-carboxamide

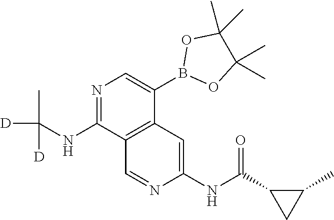 [SMILES: [2H]C([2H])(C)Nc1ncc(B2OC(C)(C)C(C)(C)O2)c2cc(NC(=O)[C@H]3C[C@H]3C)ncc12]

A mixture of (1,2R)—N-(5-bromo-8-((ethyl-1,1-d₂ amino)-2,7-naphthyridin-3-yl)-2-methyl cyclopropane-1-carboxamide (123 mg, 350 μmol), 4,4,5,5-tetramethyl-2-(4,4,5,5-tetramethyl-1,3,2-dioxaborolan-2-yl)-1,3,2-dioxaborolane (889 mg, 3.50 mmol), Pd(dppf)Cl₂ (102 mg, 0.14 mmol) and AcOK (171 mg, 1.75 mmol) in dioxane (5 mL) was degassed and purged with N₂ for 3 times, and then the mixture was stirred at 80° C. for 16 hrs under N₂ atmosphere. The reaction mixture was cooled down to 25° C. and then filtered. The filtration was concentrated under reduced pressure. The residue was purified by flash silica gel chromatography (ISCO®; 12 g SepaFlash® Silica Flash Column, Fluent of 0-80% Ethyl acetate/Petroleum ether gradient @ 30 mL/min) to afford (1S,2R)—N-(8-((ethyl-1,1-d₂)amino)-5-(4,4,5,5-tetramethyl-1,3,2-dioxaborolan-2-yl)-2,7-naphthyridin-3-yl)-2-methylcyclopropane-1-carboxamide (65 mg, 28.4% yield) as black oil. LCMS (ESI) m/z 399.1 [M+H]⁺.

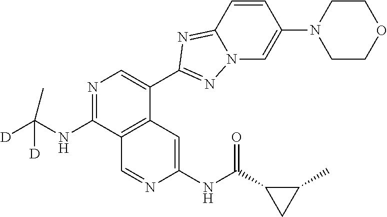 [SMILES: [2H]C([2H])(C)Nc1ncc(-c2nc3ccc(N4CCOCC4)cn3n2)c2cc(NC(=O)[C@H]3C[C@H]3C)ncc12]

Step 5: Synthesis of (1S,2R)—N-(8-((ethyl-1,1-d₂)amino)-5-(6-morpholino-[1,2,4]triazolo[1,5-a]pyridine-2-yl)-2,7-naphthyridin-3-yl)-2-methylcyclopropane-1-carboxamide was synthesized using a similar procedure that was previously described in Example 471 by using (1S,2R)—N-(8-((ethyl-1,1-d₂)amino)-5-(4,4,5,5-tetramethyl-1,3,2-dioxaborolan-2-yl)-2,7-naphthyridin-3-yl)-2-methylcyclopropane-1-carboxamide and 4-(2-chloro-[1,2,4]triazolo[1,5-a]pyridin-6-yl)morpholine as the starting material. LCMS (ESI) m/z 475.0 [M+H]⁺; ¹H NMR (400 MHz, DMSO-d₆) δ ppm 10.78 (s, 1H), 9.43 (s, 1H), 9.37 (s, 1H), 8.68 (s, 1H), 8.34 (d, J=2.0 Hz, 1H), 8.15 (s, 1H), 7.75 (br d, J=9.6 Hz, 1H), 7.67 (dd, J=10.0 Hz, J=2.0 Hz, 1H), 3.77-3.81 (m, 4H), 3.13-3.19 (m, 4H), 2.03-2.10 (m, 1H), 1.25 (s, 3H), 1.12 (d, J=6.0 Hz, 3H), 0.94-1.01 (m, 1H), 0.79-0.87 (m, 2H).

### Example 1096: Synthesis of N-(8-((ethyl-1,1-d₂)amino)-5-(6-morpholino-[1,2,4]triazolo[1,5-a]pyridin-2-yl)-2,7-naphthyridin-3-yl)cyclopropanecarboxamide

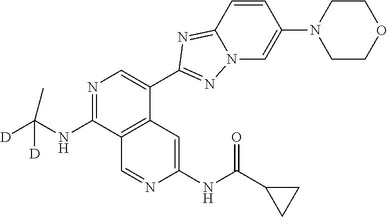 [SMILES: [2H]C([2H])(C)Nc1ncc(-c2nc3ccc(N4CCOCC4)cn3n2)c2cc(NC(=O)C3CC3)ncc12]

Example 1096 was synthesized using a similar procedure that was previously described in Example 1095 by using N-(8-((ethyl-1,1-d₂)amino)-5-(4,4,5,5-tetramethyl-1,3,2-dioxaborolan-2-yl)-2,7-naphthyridin-3-yl)cyclopropanecarboxamide and 4-(2-bromo-[1,2,4]triazolo[1,5-a]pyridin-6-yl)morpholine as the starting material. LCMS (ESI) m/z 461.0 [M+H]⁺; ¹H NMR (400 MHz, DMSO-d₆) δ ppm 10.94 (s, 1H), 9.44 (s, 1H), 9.39 (s, 1H), 8.70 (s, 1H), 8.34 (d, J=1.6 Hz, 1H), 8.17 (s, 1H), 7.76 (br d, J=1.0.0 Hz, 1H), 7.68 (dd, J=9.6 Hz, 0.12=2.0 Hz, 1H), 3.76-3.83 (m, 4H), 3.13-3.20 (m, 4H), 2.04-2.13 (m, 1H), 1.26 (s, 3H), 0.79-0.88 (m, 4H).

### Example 1097: Synthesis of 1-cyclopropyl-3-(5-(5-methoxybenzo[d]oxazol-2-yl)-8-(methylamino)-2,7-naphthyridin-3-yl)urea

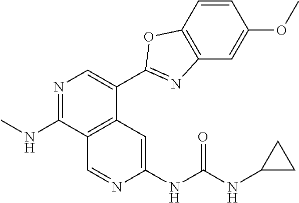

Synthesis of 1-cyclopropyl-3-(5-(5-methoxybenzo[d]oxazol-2-yl)-8-(methylamino)-2,7-naphthyridin-3-yl)urea was synthesized by using a similar procedure that was previously described in Example 1057 by using 4-(5-methoxy-1,3-benzoxazol-2-yl)-N₁-methyl-2,7-naphthyridine-1,6-diamine and isocyanatocyclopropane as the starting material. LCMS (ESI) m/z 404.9 [M+H]⁺. ; ¹H NMR (400 MHz, DMSO-d₆) δ ppm 10.65-10.84 (m, 1H), 9.72 (br s, 1H), 9.65 (s, 1H), 9.37 (s, 1H), 8.36 (s, 1H), 7.69 (d, J=8.78 Hz, 1H), 7.58 (br s, 1H), 7.33 (d, J=2.51 Hz, 1H), 7.04 (dd, J=8.78, 2.51 Hz, 1H), 3.86 (s, 3H), 3.23 (d, J=4.27 Hz, 3H), 2.65 (br dd, J=6.27, 3.51 Hz, 1H), 0.66-0.74 (m, 2H), 0.44-0.50 (m, 2H).

### Example 1098

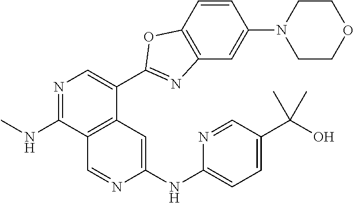 [SMILES: CNc1ncc(-c2nc3cc(N4CCOCC4)ccc3o2)c2cc(Nc3ccc(C(C)(C)O)cn3)ncc12]

Synthesis of 2-(6-((8-(methylamino)-5-(5-morpholinobenzo[d]oxazol-2-yl)-2,7-naphthyridin-3- was synthesized using a similar procedure that was previously described in Example 1086 by using N₁-methyl-4-(5-morpholino-1,3-benzoxazol-2-yl)-2,7-naphthyridine-1,6-diamine and 2-(6-bromo-3-pyridyl)propan-2-ol as the starting material. LCMS (ESI) m/z 512.0 [M+H]⁺; ¹H NMR (400 MHz, DMSO-d₆) δ ppm 9.99 (s, 1H), 9.68 (s, 1H), 9.34 (s, 1H), 8.82 (s, 1H), 8.47 (d, J=2.26 Hz, 2H), 7.79 (dd, J=8.66, 2.38 Hz, 1H), 7.60 (d, J=8.78 Hz, 1H), 7.41 (d, J=8.78 Hz, 1H), 7.25 (d, J=2.26 Hz, 1H), 7.08 (dd, J=8.91, 2.38 Hz, 1H), 5.10 (s, 1H), 3.74-3.83 (m, 3.13-3.19 (m, 4H), 3.07 (d, J=4.27 Hz, 3H), 1.49 (s, 6H).

### Example 1099

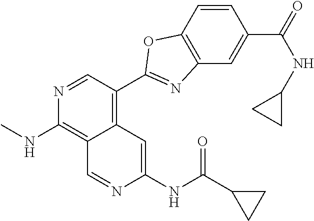 [SMILES: CNc1ncc(-c2nc3cc(C(=O)NC4CC4)ccc3o2)c2cc(NC(=O)C3CC3)ncc12]

### Synthesis of 2-(6-(cyclopropanecarboxamido)-1-(methylamino)-2,7-naphthyridin-4-yl)-N-cyclopropylbenzo[d]oxazole-5-carboxamide

To a solution of Example 214, 2-[6-(cyclopropanecarbonylamino)-1-(methylamino)-2, -naphthyridin-4-yl]-1,3-benzoxazole-5-carboxylic acid (40 mg, 0.0991 mmol), TCFH (42.0 mg, 0.149 mmol) and NMI (30.9 mg, 0.376 mmol) in DMF (3 mL) was added cyclopropanamine (16.4 mg, 0.288 mmol) and the mixture was stirred at 20° C. for 2 hr. The reaction mixture was filtered, and the filtrate was removed for reverse phase preparation and separation. The resulting residue was purified by prep-HPLC (column: 2_Phenomenex Gemini C18 75×40 mm×3 μm; mobile phase: [water (HCl)-ACN] to provide compound 2-[6-(cyclopropanecarbonylamino)-1-(methylamino)-2,7-naphthyridin-4-yl]-N-cyclopropyl-1,3-benzoxazole-5-carboxamide (8.4 mg, 18.9% yield) as a yellow solid. LCMS (ESI) m/z 443.0 [M+H]⁺; ¹H NMR (400 MHz, DMSO-d₆) δ ppm 11.13 (s, 1H), 9.77 (s, 1H), 9.48 (s, 8.90 (s, 1H), 8.63 (d, J=4.02 Hz, 1H), 8.26 (s, 1H), 7.88-7.96 (m, 1H), 7.80 (d, J=8.28 Hz, 1H), 3.12 (d, J=4.02 Hz, 3H), 2.89-2.94 (m, 1H), 2.07-2.18 (m, 1H), 0.92 (br t, J=4.89 Hz, 2H), 0.86-0.90 (m, 2H), 0.68-0.78 (m, 2H), 0.60-0.66 (m, 2H).

### Example 1100 and 1101

Table 35: the compounds were synthesized in a similar procedure as in Example 1099

| | | |
|---|---|---|
| Example 1100 | 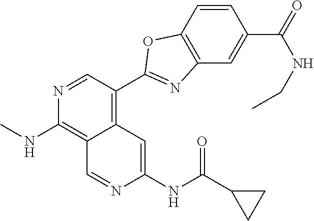 [SMILES: CCNC(=O)c1ccc2oc(-c3cnc(NC)c4cnc(NC(=O)C5CC5)cc34)nc2c1] | LCMS (ESI) m/z 431.1 [M + H]⁺; ¹H NMR (400 MHz, DMSO-d₆) δ ppm 10.85 (br s, 1H), 9.69 (s, 1H), 9.49 (s, 1H), 8.87 (s, 1H), 8.42 (br d, J = 3.76 Hz, 1H), 8.27 (d, J = 1.26 Hz, 1H), 7.94 (dd, J = 8.53, 1.51 Hz, 1H), 7.76 (d, J = 8.53 Hz, 1H), 3.35-3.38 (m, 2H), 3.16 (s, 3H), 2.07-2.19 (m, 1H), 1.19 (t, J = 7.15 Hz, 3H), 0.92-0.97 (m, 2H), 0.85-0.90 (m, 2H). |
| | 2-(6-(cyclopropanecarboxamido)-1- | |
| | (methylamino)-2,7-naphthyridin-4-yl)- | |
| | N-ethylbenzo[d]oxazole-5- | |
| | carboxamide | |
| Example 1101 | 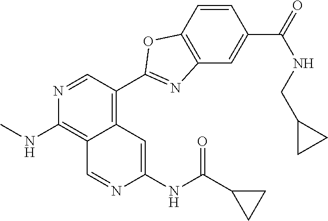 [SMILES: CNc1ncc(-c2nc3cc(C(=O)NCC4CC4)ccc3o2)c2cc(NC(=O)C3CC3)ncc12] | LCMS (ESI) m/z 457.2 [M + H]⁺; ¹H NMR (400 MHz, DMSO-d₆) δ ppm 11.12 (br d, J = 1.3 Hz, 1H), 9.79 (s, 1H), 9.50 (s, 1H), 8.87 (s, 1H), 8.78 (t, J = 5.5 Hz, 1H), 8.31 (d, J = 1.3 Hz, 1H), 7.96 (m, 1H), 7.81 (d, J = 8.3 Hz, 1H), 3.17-3.21 (m, 2H), 3.11-3.14 (m, 3H), 2.07- 2.16 (m, 1H), 1.02-1.13 (m, 1H), 0.90-0.94 (m, 2H), 0.86-0.90 (m, 2H), 0.44-0.50 (m, 2H), 0.26 (m, 2H). |
| | 2-(6-(cyclopropanecarboxamido)-1- | |
| | (methylamino)-2,7-naphthyridin-4-yl)- | |
| | N-(cyclopropylmethyl) | |
| | benzo[d]oxazole- | |
| | 5-carboxamide | |

### Example 1102

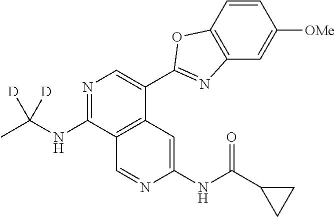 [SMILES: [2H]C([2H])(C)Nc1ncc(-c2nc3cc(OC)ccc3o2)c2cc(NC(=O)C3CC3)ncc12]

Synthesis of N-(8-((ethyl-1,l-rig)amino)-5-(5-methoxybenzo[d]oxazol-2-yl)-2,7-naphthyridin-3-yl)cyclopropanecarboxamide was synthesized using a similar procedure that was previously described in Example 916 by using N-(5-bromo-8-((ethyl-1,1-d₂)amino)-2,7-naphthyridin-3-yl)cyclopropanecarboxamide and 5-methoxy-1,3-benzoxazole as the starting material. LCMS (ESI) m/z 406.0 [M+H]⁺; ¹H NMR (400 MHz, DMSO-d₆) δ ppm, 11.05 (s, 1H), 9.64 (s, 1H), 9.46 (s, 1H), 8.86 (s, 1H), 8.57 (s, 1H), 7.61 (d, J=8.8 Hz, 1H), 7.23 (br s, 1H), 6.94 (d, J=8.8 Hz, 1H), 3.84 (s, 3H), 2.08-2.09 (m, 1H), 1.25 (s, 3H), 0.85-0.90 (m, 4H).

### Example 1103: Synthesis of 4-((6-((5-(furo[2,3-c]pyridin-2-yl)-8-(methylamino)-2,7-naphthyridin-3-yl)amino)pyridin-2-yl)oxy)butan-1-ol

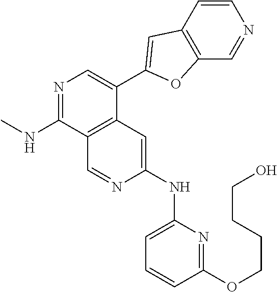 [SMILES: CNc1ncc(-c2cc3ccncc3o2)c2cc(Nc3cccc(OCCCCO)n3)ncc12]

### Step 1: Synthesis to 4-bromo-3-((4-methoxybenzyl)oxy)pyridine

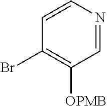

A mixture of 4-bromopyridin-3-ol (5.00 g; 28.7 mmol), 1-(chloromethyl)-4-methoxy-benzene (5.77 g, 36.8 mmol), K₂CO₃ (10.0 g, 72.4 mmol) in DMF (50 mL) was degassed and purged with N₂ for 3 times, and then the mixture was stirred at 20° C. for 12 hrs under N₂ atmosphere. The reaction mixture was quenched by addition H₂O (1.00 mL) at 25° C., extracted with EtOAc (100 mL×3). The combined organic layers were washed with brine (100 mL×3), dried over Na₂SO₄, filtered and concentrated under reduced pressure to give a residue. The residue was purified by flash chromatography (ISCO®; 40 g SepaFlash® Silica Flash Column, petroleum ether/EtOAc with EtOAc from 0-40%, flow rate: 65 mL/min, 254 nm), to provide 4-bromo-3-[(4-methoxyphenyl)methoxy]pyridine (2.70 g, 31.9% yield) as a brown solid. LCMS (ESI) m/z 293.7/295.7 [M+H]⁺.

### Step 2: Synthesis to N-(5-((3-((4-methoxybenzyl)oxy)pyridin-4-yl)ethynyl)-8-(methylamino)-2,7-naphthyridin-3-yl)cyclopropanecarboxamide

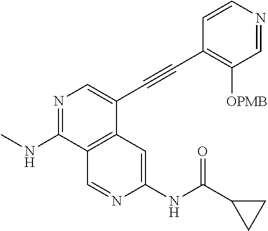

N-[5-ethynyl-8-(methylamino)-2,7-naphthyridin-3-yl]cyclopropanecarboxamide (150 mg, 0.563 mmol), 4-bromo-3-[(4-methoxyphenyl)methoxy]pyridine (270 mg, 0.918 mmol), CuI (30.0 mg, 0.158 mmol), TEA (180 mg, 1.78 mmol), XPhos Pd G₃ (105 mg, 0.124 mmol), XPhos (60.0 mg, 0.126 mmol) were taken up into a microwave tube in DMF (4 mL). The sealed tube was heated at 100° C. for 1 hr under microwave reactor. The reaction mixture was quenched by addition H₂O (100 mL) at 25° C., extracted with EtOAc (100 mL×3). The combined organic layers were washed with brine (100 mL×3), dried over anhydrous Na₂SO₄, filtered and concentrated under reduced pressure. The residue was purified by flash chromatography (ISCO®; 20 g SepaFlash® Silica Flash Column, DCM/MeOH with MeOH from 0-10%, flow rate: 40 mL/min, 254 nm), to provide N-[5-[3-[(4-methoxyphenyl)methoxy]-4-pyridyl]ethynyl]-8-(methylamino)-2,7-naphthyridin-3-yl]cyclopropanecarboxamide (200 mg, 74.0% yield) as a yellow solid. LCMS (ESI) m/z 480.0 [M+H]⁺.

### Step 3: Synthesis to 4((3-((4-methoxybenzyl)oxy)pyridin-4-yl)ethynyl)-N1-methyl-2,7-naphthyridine-1,6-diamine

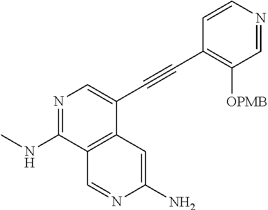

A mixture of N-[5-[2-[3-[(4-methoxyphenyl)methoxy]-4-pyridyl]ethynyl]-8-(methyl amino)-2,7-naphthyridin-3-yl]cyclopropanecarboxamide (500 mg, 1.04 mmol), 2M aq. NaOH (2.50 mL, 5.0 mmol) in MeOH (20 mL) was degassed and purged with N₂ for 3 times, and then the mixture was stirred at 80° C. for 2 hr under N₂ atmosphere. The reaction mixture was quenched by addition H₂O (50 mL) at 25° C., extracted with EtOAc (50 mL×3). The combined organic layers were washed with brine (50 mL×3), dried over Na₂SO₄, filtered and concentrated under reduced pressure to furnish 4-[2-[3-[(4-methoxyphenyl)methoxy]-4-pyridyl]ethynyl]-N1-methyl-2,7-naphthyridine-1,6-diamine (180 mg, crude) as a yellow solid. LCMS (ESI) m/z 412.0 [M+H]⁺.

### Step 4: Synthesis to 4-((6-((5-((3-((4-methoxybenzyl)oxy)pyridin-4-yl)ethynyl)-8-(methylamino)-2,7-naphthyridin-3-yl)amino)pyridin-2-yl)oxy)butan-1-ol

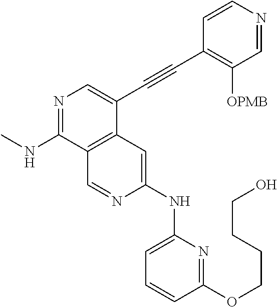

4-[2-[3-[(4-Methoxyphenyl)methoxy]-4-pyridyl]ethynyl]-N1-methyl-2,7-naphthyridine-1,6-diamine (150 mg, 0.365 mmol), 4-[(6-bromo-2-pyridyl)oxy]butan-1-ol (135 mg, 0.549 mmol), Pd₂(dba)₃ (38.0 mg, 41.5 mmol), DavePhos (30.0 mg, 76.2 mop, Cs₂CO₃ (300 mg, 0.921 mmol) were taken up into a microwave tube in DMF (5 mL). The sealed tube was heated at 120° C. for 2 hrs under microwave reactor. The reaction mixture was quenched by addition H₂O (100 mL) at 25° C., extracted with EtOAc (100 ml×3). The combined organic layers were washed with brine (1.00 mL×3), dried over Na₂SO₄, filtered and concentrated under reduced pressure to give a residue. The residue was purified by flash chromatography (ISCO®; 20 g SepaFlash® Silica Flash Column, DCM/MeOH with MeOH from 0-10%, flow rate: 65 mL/min, 254 nm), to provide 4-[[6-[[5-[2-[3-[(4-methoxyphenyl)methoxy]-4-pyridyl]ethynyl]-8-(methyl amino)-2,7-naphthyridin-3-yl]amino]-2-pyridyl]oxy]butan-1-ol (150 mg, 71.4% yield) as a brown solid. LCMS (ESI) m/z 599.1. [M°Na]⁺.

### Step 5: Synthesis of 4-((6-((5-(furo[2,3-c]pyridin-2-yl)-8-(methylamino)-2,7-naphthyridin-3-yl)amino)pyridin-2-yl)oxy)butan-1-ol

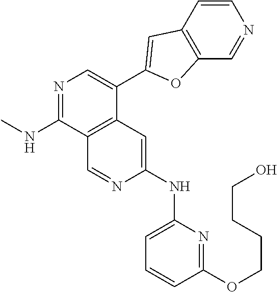 [SMILES: CNc1ncc(-c2cc3ccncc3o2)c2cc(Nc3cccc(OCCCCO)n3)ncc12]

A mixture of 4-[[6-[[5-[2-[3-[(4-methoxyphenyl)methoxy]-4-pyridyl]ethynyl]-8-(methylamino)-2,7-naphthyridin-3-yl]amino]-2-pyridyl]oxy]butan-1-ol (100 mg, 0.173 mmol), 48 wt % HBr/H₂O (3.92 g, 23.3 mmol) was degassed and purged with N₂ for 3 times, and then the mixture was stirred at 20° C. for 1 hr under N₂ atmosphere. The mixture was added water (50 mL) and sodium bicarbonate in aqueous solution until pH˜7, then extracted with EtOAc (1.00 mL×3). The combined organic layers were washed with brine (100 mL×3), dried over anhydrous Na₂SO₄, filtered and concentrated under reduced pressure. The crude product was purified by reversed-phase HPLC (Column: SepaFlash® Sphercial C18, 25 g, 40-60 μm, 120 Å; MeCN/water (0.5% NH₃—H₂O) with MeCN from 0-50%, 25 mL/min, 254 nm) to provide 4-((6-((5-(furo[2,3-c]pyridin-2-yl)-8-(methyl amino)-2,7-naphthyridin-3-yl)amino)pyridin-2-yl)oxy)butan-1-ol (3 mg, 3.8% yield) as a yellow solid. LCMS (ESI) m/z 456.9 [M+H]⁺; ¹H NMR (400 MHz, DMSO-d₆) δ ppm 9.98 (s, 1H), 9.36 (s, 1H), 8.91 (s, 1H), 8.54 (s, 1H), 8.37-8.44 (m, 2H), 8.32 (br d, J=5.0 Hz, 1H),7.70 (d, J=5.0 Hz, 1H), 7.58 (t, J=7.9 Hz, 1H), 7.24 (s, 1H), 6.97 (d, J=8.0 Hz, 1H), 6.26 (d, J=7.8 Hz, 1H), 4.30 (t, J=5.1 Hz, 1H), 3.72-3.77 (m, 2H), 3.18 (br d, J=5.3 Hz, 2H), 3.05 (d, J=4.0 Hz, 3H), 1.10-1.17 (m, 4H).

### Example 1104: N-(8-(methylamino)-5-(6-methyloxazolo[4,5-b]pyridin-2-yl)-2,7-naphthyridin-3-yl)cyclopropanecarboxamide

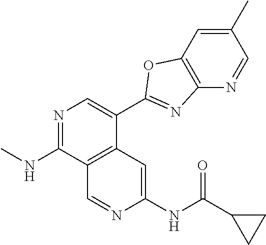 [SMILES: CNc1ncc(-c2nc3ncc(C)cc3o2)c2cc(NC(=O)C3CC3)ncc12]

Synthesis of N-(8-(methylamino)-5-(6-methyloxazolo[4,5-b]pyridin-2-yl)-2,7-naphthyridin-3-yl)cyclopropanecarboxamide was synthesized using a similar procedure that was previously described in Example 916 by using N-(5-bromo-8-(methylamino)-2,7-naphthyridin-3-yl)cyclopropanecarboxamide and 6-methyloxazolo[4,5-b]pyridine as the starting material. LCMS (ESI) m/z 397.0 [M+Na]; ¹H NMR (400 MHz, DMSO-d₆) δ ppm 11.03-11.10 (m, 1H), 9.72 (s, 1H), 9.44 (s, 1H), 8.95 (s, 1H), 8.76 (br d, J=4.4 Hz, 1H), 8.33 (s, 1H), 7.98 (d, J=0.8 Hz, 1H), 3.09 (d, J=4.4 Hz, 3H), 2.46 (s, 3H), 2.05-2.13 (m, 1H), 0.89-0.95 (m, 2H), 0.82-0.88 (m, 2H).

### Example 1105: N-(5-(5-(2-(methyl-d₃)morpholino)benzo[d]oxazol-2-yl)-8-(methylamino)-2,7-naphthyridin-3-yl)cyclopropanecarboxamide

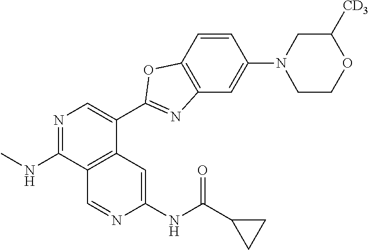 [SMILES: [2H]C([2H])([2H])C1CN(c2ccc3oc(-c4cnc(NC)c5cnc(NC(=O)C6CC6)cc45)nc3c2)CCO1]

Synthesis of N-(5-(5-(2-(methyl-d₃)morpholino)benzo[d]oxazol-2-yl)-8-(methylamino)-2,7-naphthyridin-3-yl)cyclopropanecarboxamide was synthesized using a similar procedure that was previously described in Example 916 by using N-(5-bromo-8-(methylamino)-2,7-naphthyridin-3-yl)cyclopropanecarboxamide and 5-(2-(methyl-d₃)morpholino)benzo[d]oxazole as the starting material. LCMS (ESI) m/z 462.1 [M+H]⁺; ¹H NMR (400 MHz, DMSO-d₆) δ ppm 11.03 (s, 1H), 9.64 (s, 1H), 9.43 (s, 1H), 8.88 (s, 1H), 8.62 (br d, J=4.4 Hz, 1H), 7.57 (d, J=9.2 Hz, 1H), 7.22 (d, J=2.4 Hz, 1H), 7.06 (dd, J=8.8, 2.4 Hz, 1H), 3.93 (br d, J=9.2 Hz, 1H), 3.66-3.71 (m, 2H), 3.58-3.63 (m, 2H), 3.08 (d, J=4.4 Hz, 3H), 2.67-2.74 (m, 1H), 2.35-2.42 (m, 1H), 2.06-2.14 (m, 1H), 0.83-0.92 (m, 4H).

### Example 1106: Synthesis of N-(5-(5-(6-oxa-3-azabicyclo[3.1.1]heptan-3-yl)benzo[d]oxazol-2-yl)-8-(methylamino)-2,7-naphthyridin-3-yl)cyclopropanecarboxamide

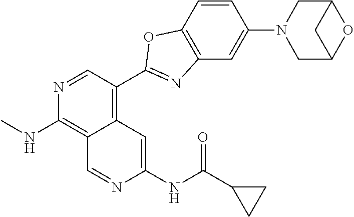 [SMILES: CNc1ncc(-c2nc3cc(N4CC5CC(C4)O5)ccc3o2)c2cc(NC(=O)C3CC3)ncc12]

A mixture of N-[5-(5-bromo-1,3-benzoxazol-2-yl)-8-(methylamino)-2,7-naphthyridin-3-yl]cyclopropanecarboxamide, Example 209, (250 mg, 0.57 mmol, 1 eq), 6-oxa-3-azabicyclo[3.1.1]heptane (309.37 mg, 2.28 mmol, 10 eq, HCl), Cs₂CO₃ (1.12 g, 3.42 mmol, 6 eq), RuPhos-Pd-G₃ (95.42 mg, 0.11 mmol, 0.2 eq) in dioxane (2 mL) was degassed and purged with N₂ for 3 times, and then the mixture was stirred at 110° C. for 16 hrs under N₂ atmosphere. The reaction mixture was concentrated under reduced pressure to remove solvent. The residue was purified by prep-HPLC (column: Welch Xtimate C18 150×25 mm×5 μm; mobile phase: [water (NH₃H₂O)-ACN]; gradient: 30%-60% B over 9.5 min); to provide N-(5-(5-(6-oxa-3-azabicyclo[3.1.1]heptan-3-yl)benzo[d]oxazol-2-yl)-8-(methylamino)-2,7-naphthyridin-3-yl)cyclopropanecarboxamide (3.85 mg, 1.42% yield, 96.30% purity) as yellow solid. LCMS (ESI) m/z 457.1 [M+H]⁺; ¹H NMR (400 MHz, DMSO-d₆) δ ppm 11.00-11.06 (m, 1H), 9.69 (s, 1H), 9.44 (s, 1H), 8.88-8.93 (m, 1H), 8.60-8.67 (m, 1H), 7.59 (d, J=8.8 Hz, 1H), 7.00 (d, J=2.4 Hz, 1H), 6.83 (dd, J=9.2, 2.4 Hz, 1H), 4.75 (br d, J=6.41 Hz, 2H), 3.65 (d, J=11.2 Hz, 2H), 3.46 (br d, J=10.8 Hz, 2H), 3.11-3.18 (m, 1H), 3.10 (br s, 1H), 3.09 (s, 2H), 2.07-2.16 (m, 1H), 1.92-2.03 (m, 1H), 0.90-0.93 (m, 2H), 0.86 (hr dd, J=7.6, 3.14 Hz, 2H).

### Examples 1107-1137

Each compound in Tables 35A and 36 were prepared using a similar experimental procedure to prepare Example 950, using N-(5-ethynyl-8-(methylamino)-2,7-naphthyridin-3-yl)cyclopropanecarboxamide as the common intermediate and appropriate aryl halides.

TABLE 35A

| Example | Structure and Name | MS and ¹H NMR |
|---|---|---|
| Example 1107 | 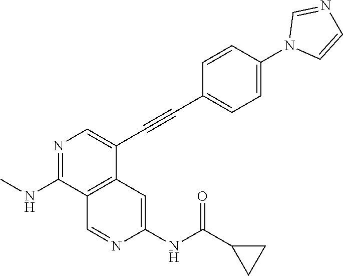 [SMILES: CNc1ncc(C#Cc2ccc(-n3ccnc3)cc2)c2cc(NC(=O)C3CC3)ncc12] | LCMS (ESI) m/z 409.1 [M + H]⁺; ¹H NMR (400 MHz, DMSO-d₆) δ ppm 11.12 (s, 1H), 9.39 (s, 1H), 8.69 (s, 1H), 8.36-8.47 (m, 2H), 8.30 (s, 1H), 7.87 (s, 1H), 7.78 (d, J = 8.52 Hz, 2H), 7.65-7.72 (m, 2H), 7.17 (br s, 1H), 3.00 (d, J = 4.4 Hz, 3H), 2.06-2.10 (m, 1H), 0.88-0.92 (m, 2H), 0.83-0.87 (m, 2H). |
| | N-(5-((4-(1H-imidazol-1- | |
| | yl)phenyl)ethynyl)-8- | |
| | (methylamino)-2,7-naphthyridin- | |
| | 3-yl)cyclopropanecarboxamide | |
| Example 1108 | 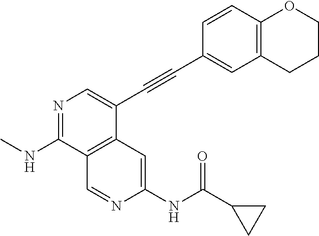 [SMILES: CNc1ncc(C#Cc2ccc3c(c2)CCCO3)c2cc(NC(=O)C3CC3)ncc12] | LCMS (ESI) m/z: 399.1 [M + H]⁺; ¹H NMR (400 MHz, DMSO-d₆) δ ppm 11.08 (s, 1H), 9.35 (s, 1H), 8.64 (s, 1H), 8.27 (br d, J = 4.4 Hz, 1H), 8.21 (s, 1H), 7.32 (d, J = 4.0 Hz, 1H), 7.24 (dd, J = 8.4, 1.88 Hz, 1H), 6.78 (d, J = 8.8 Hz, 1H), 4.13-4.22 (m, 2H), 3.00 (d, J = 4.4 Hz, 3H), 2.76 (t, J = 6.4 Hz, 2H), 2.03-2.11 (m, 1H), 1.90-1.97 (m, 2H), 0.80-0.91 (m, 4H). |
| | N-(5-(chroman-6-ylethynyl)-8- | |
| | (methylamino)-2,7-naphthyridin- | |
| | 3-yl)cyclopropanecarboxamide | |
| Example 1109 | 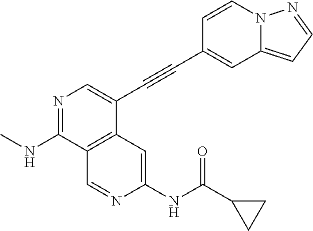 [SMILES: CNc1ncc(C#Cc2ccn3nccc3c2)c2cc(NC(=O)C3CC3)ncc12] | LCMS (ESI) m/z 383.1 [M + H]⁺; ¹H NMR (400 MHz, DMSO-d₆) δ ppm 11.13 (s, 1H), 9.38 (s, 1H), 8.74 (d, J = 7.2 Hz, 1H), 8.67 (s, 1H), 8.41 (br d, J = 4.8 Hz, 1H), 8.32 (s, 1H), 8.06 (d, J = 2.4 Hz, 1H), 7.91 (s, 1H), 6.94 (dd, J = 7.2, 1.6 Hz, 1H), 6.69 (d, J = 1.6 Hz, 1H), 3.03 (d, J = 4.4 Hz, 3H), 2.05- 2.12 (m, 1H), 0.91 (dd, J = 4.4, 2.4 Hz, 2H), 0.86 (dt, J = 8.0, 2.8 Hz, 2H). |
| | N-(8-(methylamino)-5- | |
| | (pyrazolo[1,5-a]pyridin-5- | |
| | ylethynyl)-2,7-naphthyridin-3- | |
| | yl)cyclopropanecarboxamide | |
| Example 1110 | 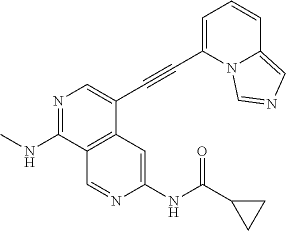 [SMILES: CNc1ncc(C#Cc2cccc3cncn23)c2cc(NC(=O)C3CC3)ncc12] | LCMS (ESI) m/z: 383.1 [M + H]⁺; ¹H NMR (400 MHz, DMSO-d₆) δ ppm 11.18 (s, 1H), 9.41 (s, 1H), 8.62-8.73 (m, 2H), 8.53-8.57 (m, 1H), 8.51 (s, 1H), 7.69 (d, J = 8 Hz, 1H), 7.57 (s, 1H), 7.03 (d, J = 6.4 Hz, 1H), 6.90 (br d, J = 8 Hz, 1H), 3.05 (d, J = 4.4 Hz, 3H), 2.03-2.13 (m, 1H), 0.82-0.92 (m, 4H). |
| | N-(5-(imidazo[1,5-a]pyridin-5- | |
| | ylethynyl)-8-(methylamino)-2,7- | |
| | naphthyridin-3- | |
| | yl)cyclopropanecarboxamide | |
| Example 1111 | 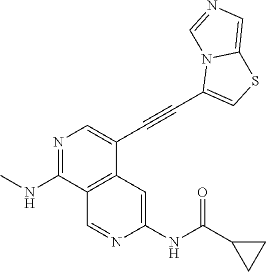 [SMILES: CNc1ncc(C#Cc2csc3cncn23)c2cc(NC(=O)C3CC3)ncc12] | LCMS (ESI) m/z: 389.0 [M + H]⁺; ¹H NMR (400 MHz, DMSO-d₆) δ ppm 11.19 (s, 1H), 9.40 (s, 1H), 8.62 (s, 1H), 8.53 (br d, J = 4.4 Hz, 1H), 8.42 (s, 2H), 7.64 (s, 1H), 7.17 (br s, 1H), 3.04 (d, J = 4.4 Hz, 3H), 2.07 (m, 1H), 0.83-0.91 (m, 4H). |
| | N-(5-(imidazo[5,1-b]thiazol-3- | |
| | ylethynyl)-8-(methylamino)-2,7- | |
| | naphthyridin-3- | |
| | yl)cyclopropanecarboxamide | |
| Example 1112 | 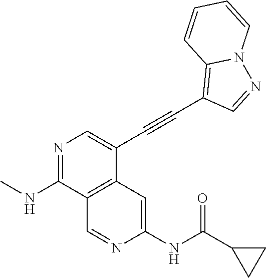 [SMILES: CNc1ncc(C#Cc2cnn3ccccc23)c2cc(NC(=O)C3CC3)ncc12] | LCMS (ESI) m/z: 383.1 [M + H]⁺; ¹H NMR (400 MHz, DMSO-d₆) δ ppm 11.13 (s, 1H), 9.38 (s, 1H), 8.79 (d, J = 8.0 Hz, 1H), 8.74 (s, 1H), 8.28 (d, J = 4.0 Hz, 3H), 8.09 (d, J = 8.8 Hz, 1H), 7.39 (dd, J = 8.8, 1.2 Hz, 1H), 7.05 (td, J = 6.8, 1.2 Hz, 1H), 3.01 (d, J = 4.0 Hz, 3H), 2.06-2.12 (m, 1H), 0.85- 0.91 (m, 4H). |
| | N-(8-(methylamino)-5- | |
| | (pyrazolo[1,5-a]pyridin-3- | |
| | ylethynyl)-2,7-naphthyridin-3- | |
| | yl)cyclopropanecarboxamide | |
| Example 1113 | 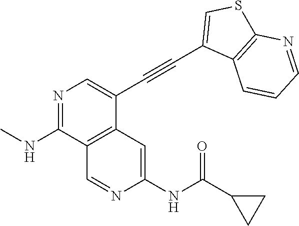 [SMILES: CNc1ncc(C#Cc2csc3ncccc23)c2cc(NC(=O)C3CC3)ncc12] | LCMS (ESI) m/z 399.9 [M + H]⁺; ¹H NMR (400 MHz, DMSO-d₆) δ ppm 11.20 (s, 1H), 9.41 (s, 1H), 8.79 (s, 1H), 8.64-8.74 (m, 2H), 8.34-8.49 (m, 2H), 8.21 (s, 1H), 7.56 (dd, J = 8.8, 4.4 Hz, 1H), 3.04 (d, J = 4.4 Hz, 3H), 2.12 (m, 1H), 0.94 (br s, 2H), 0.89 (br d, J = 8.0 Hz, 2H). |
| | N-(8-(methylamino)-5- | |
| | (thieno[2,3-b]pyridin-3- | |
| | ylethynyl)-2,7-naphthyridin-3- | |
| | yl)cyclopropanecarboxamide | |
| Example 1115 | 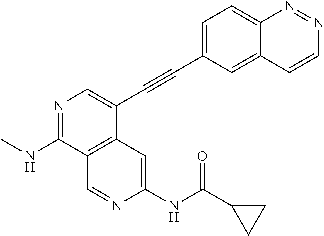 [SMILES: CNc1ncc(C#Cc2ccc3nnccc3c2)c2cc(NC(=O)C3CC3)ncc12] | LCMS (ESI) m/z 395.0 [M + H]⁺; ¹H NMR (400 MHz, DMSO-d₆) δ ppm 11.15 (s, 1H), 9.31-9.47 (m, 2H), 8.73 (s, 1H), 8.43-8.54 (m, 2H), 8.39 (s, 1H), 8.27 (s, 1H), 8.19 (d, J = 5.6 Hz, 1H), 8.02 (dd, J = 8.8, 1.6 Hz, 1H), 3.04 (d, J = 4.4 Hz, 3H), 2.03-2.15 (m, 1H), 0.93 (br d, J = 4.4 Hz, 2H), 0.72- 0.89 (m, 2H). |
| | N-(5-(cinnolin-6-ylethynyl)-8- | |
| | (methylamino)-2,7-naphthyridin- | |
| | 3-yl)cyclopropanecarboxamide | |
| Example 1116 | 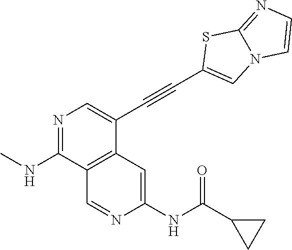 [SMILES: CNc1ncc(C#Cc2cn3ccnc3s2)c2cc(NC(=O)C3CC3)ncc12] | LCMS (ESI) m/z 389.0 [M + H]⁺; ¹H NMR (400 MHz, DMSO-d₆) δ ppm 11.11 (s, 1H), 9.33-9.41 (m, 1H), 8.52 (s, 1H), 8.47 (br d, J = 4.4 Hz, 1H), 8.33 (s, 1H), 8.32 (s, 1H), 7.82 (br s, 1H), 7.35 (br s, 1H), 3.02 (d, J = 4.4 Hz, 3H), 2.03-2.12 (m, 1H), 0.81- 0.91 (m, 4H). |
| | N-(5-(imidazo[2,1-b]thiazol-2- | |
| | ylethynyl)-8-(methylamino)-2,7- | |
| | naphthyridin-3- | |
| | yl)cyclopropanecarboxamide | |

TABLE 36

| | | |
|---|---|---|
| Example 1117 | 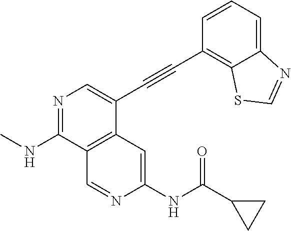 [SMILES: CNc1ncc(C#Cc2cccc3ncsc23)c2cc(NC(=O)C3CC3)ncc12] | LCMS (ESI) m/z 400.0 [M + H]⁺; ¹H NMR (400 MHz, DMSO-d₆) δ ppm 11.10 (s, 1H), 9.51 (s, 1H), 9.39 (s, 1H), 8.66 (s, 1H), 8.42-8.47 (m, 1H), 8.36 (s, 1H), 8.13 (m, 1H), 7.68-7.72 (m, 1H), 7.60-7.65 (m, 1H), 3.03 (d, J = 4.5 Hz, 3H), 2.05-2.10 (m, 1H), 0.82-0.88 (m, 4H). |
| | N-(5-(benzo[d]thiazol-7- | |
| | ylethynyl)-8-(methylamino)-2,7- | |
| | naphthyridin-3- | |
| | yl)cyclopropanecarboxamide | |
| Example 1118 | 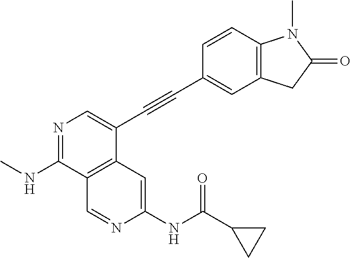 [SMILES: CNc1ncc(C#Cc2ccc3c(c2)CC(=O)N3C)c2cc(NC(=O)C3CC3)ncc12] | LCMS (ESI) m/z 412.1 [M + H]⁺; ¹H NMR (400 MHz, DMSO-d₆) δ ppm 11.14 (s, 1H), 9.34-9.42 (m, 1H), 8.72- 8.84 (m, 1H), 8.36 (br d, J = 4.5 Hz, 1H), 8.20-8.32 (m, 1H), 7.32 (d, J = 7.5 Hz, 1H), 7.28 (s, 1H), 7.20 (m, 1H), 3.61 (s, 2H), 3.18 (s, 3H), 2.99-3.05 (m, 3H), 2.09 (m, 1H), 0.82-0.91 (m, 4H). |
| | N-(5-((1-methyl-2-oxoindolin-5- | |
| | yl)ethynyl)-8-(methylamino)-2,7- | |
| | naphthyridin-3- | |
| | yl)cyclopropanecarboxamide | |
| Example 1119 | 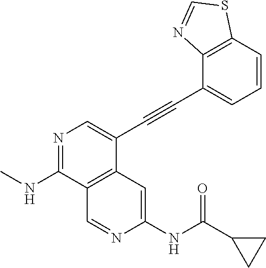 [SMILES: CNc1ncc(C#Cc2cccc3scnc23)c2cc(NC(=O)C3CC3)ncc12] | LCMS (ESI) m/z 400.1 [M + H]⁺; ¹H NMR (400 MHz, DMSO-d₆) δ ppm 11.51 (s, 1H), 10.78-10.89 (m, 1H), 9.85 (s, 1H), 9.51 (s, 1H), 8.86 (s, 1H), 8.30 (d, J = 8.0 Hz, 1H), 8.09 (s, 1H), 7.83-7.86 (m, 1H), 7.59 (t, J = 7.8 Hz, 1H), 3.20 (d, J = 4.3 Hz, 3H), 2.11 (m, 1H), 0.87-0.91 (m, 4H). |
| | N-(5-(benzo[d]thiazol-4- | |
| | ylethynyl)-8-(methylamino)-2,7- | |
| | naphthyridin-3- | |
| | yl)cyclopropanecarboxamide | |
| Example 1120 | 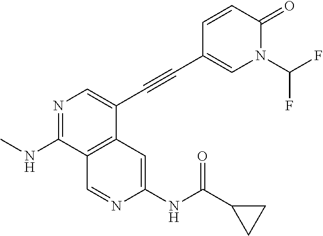 [SMILES: CNc1ncc(C#Cc2ccc(=O)n(C(F)F)c2)c2cc(NC(=O)C3CC3)ncc12] | LCMS (ESI) m/z 410.1 [M + H]⁺; ¹H NMR (400 MHz, DMSO-d₆) δ ppm 11.52 (s, 1H), 10.50 (br s, 1H), 9.72 (s, 1H), 8.80 (s, 1H), 8.25 (d, J = 2.1 Hz, 1H), 8.05 (s, 1H), 7.73-8.03 (m, 1H), 7.65 (m, 1H), 6.66 (d, J = 9.6 Hz, 1H), 3.16 (br d, J = 3.8 Hz, 3H), 2.07-2.14 (m, 1H), 0.89-0.93 (m, 4H). |
| | N-(5-((1-(difluoromethyl)-6-oxo- | |
| | 1,6-dihydropyridin-3-yl)ethynyl)- | |
| | 8-(methylamino)-2,7- | |
| | naphthyridin-3- | |
| | yl)cyclopropanecarboxamide | |
| Example 1121 | 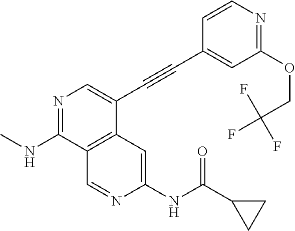 [SMILES: CNc1ncc(C#Cc2ccnc(OCC(F)(F)F)c2)c2cc(NC(=O)C3CC3)ncc12] | LCMS (ESI) m/z 442.1 [M + H]⁺; ¹H NMR (400 MHz, DMSO-d₆) δ ppm 11.16 (s, 1H), 9.40 (s, 1H), 8.68 (s, 1H), 8.32 (s, 1H), 8.26 (d, J = 5.3 Hz, 1H), 7.21 (m, 1H), 7.12 (s, 1H), 5.03 (m, 2H), 3.04 (d, J = 4.5 Hz, 3H), 2.05- 2.11 (m, 1H), 0.89-0.92 (m, 2H), 0.86 (m, 2H). |
| | N-(8-(methylamino)-5-((2-(2,2,2- | |
| | trifluoroethoxy)pyridin-4- | |
| | yl)ethynyl)-2,7-naphthyridin-3- | |
| | yl)cyclopropanecarboxamide | |
| Example 1122 | 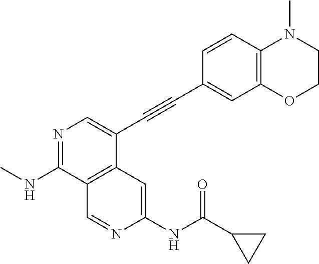 [SMILES: CNc1ncc(C#Cc2ccc3c(c2)OCCN3C)c2cc(NC(=O)C3CC3)ncc12] | LCMS (ESI) m/z 414.2 [M + H]⁺; ¹H NMR (400 MHz, CDCl₃) δ ppm 8.89 (br s, 1H), 8.79 (s, 1H), 8.32 (br s, 1H), 8.23 (br s, 1H), 7.16 (br d, J = 7.5 Hz, 1H), 7.03 (s, 1H), 6.62 (br d, J = 7.5 Hz, 1H), 5.69 (br s, 1H), 4.20-4.38 (m, 2H), 3.32 (br s, 2H), 3.20 (br s, 3H), 2.93 (br s, 3H), 2.26 (m, 1H), 1.13- 1.23 (m, 2H), 0.87-1.00 (m, 2H). |
| | N-(5-((4-methyl-3,4-dihydro-2H- | |
| | benzo[b][1,4]oxazin-7- | |
| | yl)ethynyl)-8-(methylamino)-2,7- | |
| | naphthyridin-3- | |
| | yl)cyclopropanecarboxamide | |
| Example 1123 | 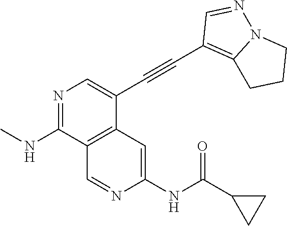 [SMILES: CNc1ncc(C#Cc2cnn3c2CCC3)c2cc(NC(=O)C3CC3)ncc12] | LCMS (ESI) m/z 373.1 [M + H]⁺; ¹H NMR (400 MHz, DMSO-d₆) δ ppm 11.49 (s, 1H), 10.44 (br d, J = 1.5 Hz, 1H), 9.69 (s, 1H), 8.76 (s, 1H), 7.97 (s, 1H), 7.72 (s, 1H), 4.14 (t, J = 7.4 Hz, 2H), 3.15 (br d, J = 4.0 Hz, 3H), 3.01 (t, J = 7.4 Hz, 2H), 2.61 (t, J = 7.4 Hz, 2H), 2.06-2.14 (m, 1H), 0.89 (d, J = 5.8 Hz, 4H). |
| | N-(5-((5,6-dihydro-4H- | |
| | pyrrolo[1,2-b]pyrazol-3- | |
| | yl)ethynyl)-8-(methylamino)-2,7- | |
| | naphthyridin-3- | |
| | yl)cyclopropanecarboxamide | |
| Example 1124 | 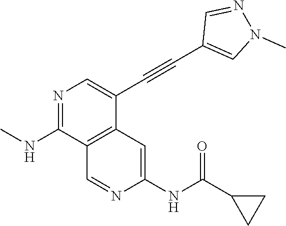 | LCMS (ESI) m/z 346.9 [M + H]⁺; ¹H NMR (400 MHz, DMSO-d₆) δ ppm 11.48 (s, 1H), 10.75 (br s, 1H), 9.81 (s, 1H), 8.71 (s, 1H), 8.13 (s, 1H), 7.96 (s, 1H), 7.75 (s, 1H), 3.89 (s, 3H), 3.16 (br d, J = 3.8 Hz, 3H), 2.06-2.17 (m, 1H), 0.90 (br s, 4H). |
| | N-(5-((1-methyl-1H-pyrazol-4- | |
| | yl)ethynyl)-8-(methylamino)-2,7- | |
| | naphthyridin-3- | |
| | yl)cyclopropanecarboxamide | |
| Example 1125 | 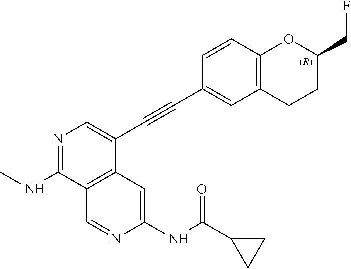 | LCMS (ESI) m/z 431.0 [M + H]⁺; ¹H NMR (400 MHz, DMSO-d₆) δ ppm 11.08 (s, 1H), 9.36 (s, 1H), 8.64 (s, 1H), 8.28 (br d, J = 4.5 Hz, 1H), 8.22 (s, 1H), 7.35 (s, 1H), 7.26 (m, 1H), 6.84 (d, J = 8.5 Hz, 0.5H), 4.70-4.78 (m, 1H), 4.60-4.68 (m, 0.5H), 4.49-4.56 (m, 1H), 4.27-4.41 (m, 1H), 3.00 (d, J = 4.3 Hz, 3H), 2.83-2.93 (m, 1H), 2.73-2.81 (m, 1H), 1.98-2.12 (m, 2H), 1.69-1.81 (m, 1H), 0.81-0.92 (m, 4H). |
| | (R)-N-(5-((2- | |
| | (fluoromethyl)chroman-6- | |
| | yl)ethynyl)-8-(methylamino)-2,7- | |
| | naphthyridin-3- | |
| | yl)cyclopropanecarboxamide | |
| Example 1126 | 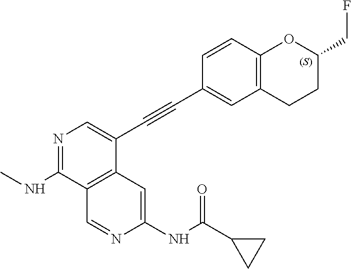 | LCMS (ESI) m/z 431.0 [M + H]⁺; ¹H NMR (400 MHz, DMSO-d₆) δ ppm 11.08 (s, 1H), 9.36 (s, 1H), 8.64 (s, 1H), 8.28 (br d, J = 4.5 Hz, 1H), 8.22 (s, 1H), 7.35 (s, 1H), 7.26 (m, 1H), 6.84 (d, J = 8.5 Hz, 0.5H), 4.70-4.78 (m, 1H), 4.60-4.68 (m, 0.5H), 4.49-4.56 (m, 1H), 4.27-4.41 (m, 1H), 3.00 (d, J = 4.3 Hz, 3H), 2.83-2.93 (m, 1H), 2.73-2.81 (m, 1H), 1.98-2.12 (m, 2H), 1.69-1.81 (m, 1H), 0.81-0.92 (m, 4H). |
| | (S)-N-(5-((2- | |
| | (fluoromethyl)chroman-6- | |
| | yl)ethynyl)-8-(methylamino)-2,7- | |
| | naphthyridin-3- | |
| | yl)cyclopropanecarboxamide | |
| Example 1127 | 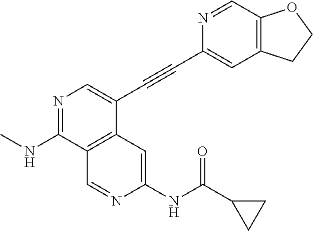 [SMILES: CNc1ncc(C#Cc2cc3c(cn2)OCC3)c2cc(NC(=O)C3CC3)ncc12] | LCMS (ESI) m/z 408.0 [M + Na]⁺; ¹H NMR (400 MHz, DMSO-d₆) δ ppm 11.09 (s, 1H), 9.37 (s, 1H), 8.60 (s, 1H), 8.33-8.39 (m, 1H), 8.29 (s, 1H), 8.13 (s, 1H), 7.58 (s, 1H), 4.64 (t, J = 8.8 Hz, 2H), 3.26 (br s, 2H), 3.02 (d, J = 4.5 Hz, 3H), 2.03-2.10 (m, 1H), 0.81-0.88 (m, 4H). |
| | N-(5-((2,3-dihydrofuro[2,3- | |
| | c]pyridin-5-yl)ethynyl)-8- | |
| | (methylamino)-2,7-naphthyridin- | |
| | 3-yl)cyclopropanecarboxamide | |
| Example 1128 | 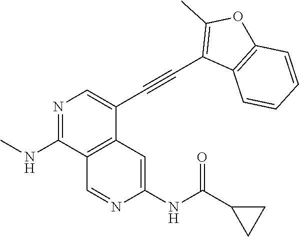 [SMILES: CNc1ncc(C#Cc2c(C)oc3ccccc23)c2cc(NC(=O)C3CC3)ncc12] | LCMS (ESI) m/z 397.1 [M + H]⁺; ¹H NMR (400 MHz, DMSO-d₆) δ ppm 11.13 (s, 1H), 9.39 (s, 1H), 8.75 (s, 1H), 8.30-8.37 (m, 2H), 7.86 (m, 1H), 7.59 (d, J = 7.5 Hz, 1H), 7.29-7.38 (m, 2H), 3.01-3.06 (m, 3H), 2.67 (s, 3H), 2.07-2.12 (m, 1H), 0.84-0.91 (m, 4H). |
| | N-(8-(methylamino)-5-((2- | |
| | methylbenzofuran-3-yl)ethynyl)- | |
| | 2,7-naphthyridin-3- | |
| | yl)cyclopropanecarboxamide | |
| Example 1129 | 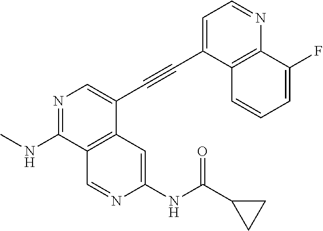 [SMILES: CNc1ncc(C#Cc2ccnc3c(F)cccc23)c2cc(NC(=O)C3CC3)ncc12] | LCMS (ESI) m/z 411.9 [M + H]⁺; ¹H NMR (400 MHz, DMSO-d₆) δ ppm 11.19 (s, 1H), 9.41 (br s, 1H), 8.95 (br s, 1H), 8.82 (s, 1H), 8.56 (br d, J = 4.3 Hz, 1H), 8.42-8.50 (m, 2H), 7.64- 7.77 (m, 3H), 3.05 (d, J = 4.0 Hz, 3H), 2.13 (m, 1H), 0.97 (m, 2H), 0.86-0.93 (m, 2H). |
| | N-(8-(methylamino)-5-((2- | |
| | methylbenzofuran-3-yl)ethynyl)- | |
| | 2,7-naphthyridin-3- | |
| | yl)cyclopropanecarboxamide | |
| Example 1130 | 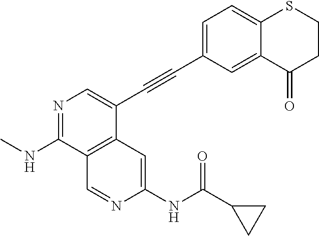 [SMILES: CNc1ncc(C#Cc2ccc3c(c2)C(=O)CCS3)c2cc(NC(=O)C3CC3)ncc12] | LCMS (ESI) m/z 429.0 [M + H]⁺; ¹H NMR (400 MHz, DMSO-d₆) δ ppm 11.09 (s, 1H), 9.37 (s, 1H), 8.64 (s, 1H), 8.36 (br d, J = 4.5 Hz, 1H), 8.31 (s, 1H), 8.07 (d, J = 2.0 Hz, 1H), 7.60 (m, 1H), 7.46 (d, J = 8.3 Hz, 1H), 3.37- 3.41 (m, 2H), 3.02 (d, J = 4.3 Hz, 3H), 2.92-2.97 (m, 2H), 2.05-2.12 (m, 1H), 0.83-0.93 (m, 4H). |
| | N-(8-(methylamino)-5-((4- | |
| | oxothiochroman-6-yl)ethynyl)- | |
| | 2,7-naphthyridin-3- | |
| | yl)cyclopropanecarboxamide | |
| Example 1131 | 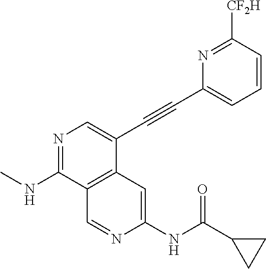 [SMILES: CNc1ncc(C#Cc2cccc(C(F)F)n2)c2cc(NC(=O)C3CC3)ncc12] | LCMS (ESI) m/z 394.0 [M + H]⁺; ¹H NMR (400 MHz, DMSO-d₆) δ ppm 11.46 (s, 1H), 10.31 (br s, 1H), 9.68 (s, 1H), 8.74 (s, 1H), 8.26 (s, 1H), 8.10- 8.19 (m, 1H), 7.87 (d, J = 7.8 Hz, 1H), 7.76 (d, J = 7.8 Hz, 1H), 6.83-7.17 (m, 1H), 3.15 (br d, J = 3.3 Hz, 3H), 2.05- 2.15 (m, 1H), 0.87-0.93 (m, 4H). |
| | N-(5-((6- | |
| | (difluoromethyl)pyridin-2- | |
| | yl)ethynyl)-8-(methylamino)-2,7- | |
| | naphthyridin-3- | |
| | yl)cyclopropanecarboxamide | |
| Example 1132 | 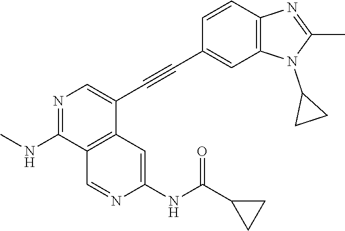 | LCMS (ESI) m/z 437.0 [M + H]⁺; ¹H NMR (400 MHz, DMSO-d₆) δ ppm 11.10 (s, 1H), 9.37 (s, 1H), 8.76 (s, 1H), 8.27-8.33 (m, 2H), 7.76 (s, 1H), 7.55 (d, J = 8.3 Hz, 1H), 7.34 (m, 1H), 3.02 (d, J = 4.3 Hz, 3H), 2.61 (s, 3H), 2.06-2.12 (m, 1H), 1.23 (br d, J = 4.5 Hz, 3H), 1.06-1.10 (m, 2H), 0.88- 0.92 (m, 2H), 0.85 (m, 2H). |
| | N-(5-((1-cyclopropyl-2-methyl- | |
| | 1H-benzo[d]imidazol-6- | |
| | yl)ethynyl)-8-(methylamino)-2,7- | |
| | naphthyridin-3- | |
| | yl)cyclopropanecarboxamide | |
| Example 1133 | 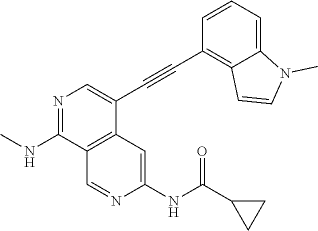 | LCMS (ESI) m/z 396.1 [M + H]⁺; ¹H NMR (400 MHz, DMSO-d₆) δ ppm 11.08-11.11 (m, 1H), 9.38 (s, 1H), 8.75 (s, 1H), 8.29-8.35 (m, 2H), 7.49 (d, J = 8.0 Hz, 1H), 7.43 (d, J = 3.0 Hz, 1H), 7.24-7.27 (m, 1H), 7.17-7.21 (m, 1H), 6.85 (d, J = 3.0 Hz, 1H), 3.84 (s, 3H), 3.03 (d, J = 4.3 Hz, 3H), 2.06- 2.11 (m, 1H), 0.90 (br d, J = 2.3 Hz, 2H), 0.84-0.88 (m, 2H). |
| | N-(5-((1-methyl-1H-indol-4- | |
| | yl)ethynyl)-8-(methylamino)-2,7- | |
| | naphthyridin-3- | |
| | yl)cyclopropanecarboxamide | |
| Example 1134 | 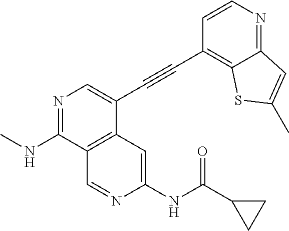 | LCMS (ESI) m/z 414.1 [M + H]⁺; ¹H NMR (400 MHz, DMSO-d₆) δ ppm 11.37 (s, 1H), 9.59 (s, 1H), 8.76 (s, 1H), 8.72-8.75 (m, 1H), 8.37 (s, 1H), 7.56 (d, J = 5.3 Hz, 1H), 7.45 (d, J = 1.0 Hz, 1H), 3.14 (br d, J = 4.0 Hz, 3H), 2.72 (d, J = 0.8 Hz, 3H), 2.12 (m, 1H), 0.88-0.93 (m, 4H). |
| | N-(8-(methylamino)-5-((2- | |
| | methylthieno[3,2-b]pyridin-7- | |
| | yl)ethynyl)-2,7-naphthyridin-3- | |
| | yl)cyclopropanecarboxamide | |
| Example 1135 | 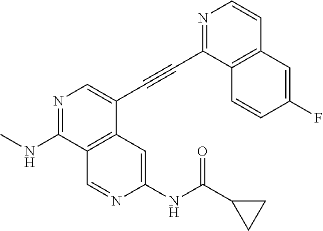 [SMILES: CNc1ncc(C#Cc2nccc3cc(F)ccc23)c2cc(NC(=O)C3CC3)ncc12] | LCMS (ESI) m/z 412.1 [M + H]⁺; ¹H NMR (400 MHz, DMSO-d₆) δ ppm 11.21 (s, 1H), 9.42 (s, 1H), 8.79-8.86 (m, 2H), 8.52-8.57 (m, 2H), 8.18 (br s, 1H), 7.81-7.88 (m, 2H), 7.62 (m, 1H), 3.06 (d, J = 4.4 Hz, 3H), 2.09- 2.18 (m, 1H), 0.86-1.00 (m, 4H). |
| | N-(5-((6-fluoroisoquinolin-1- | |
| | yl)ethynyl)-8-(methylamino)-2,7- | |
| | naphthyridin-3- | |
| | yl)cyclopropanecarboxamide | |
| Example 1136 | 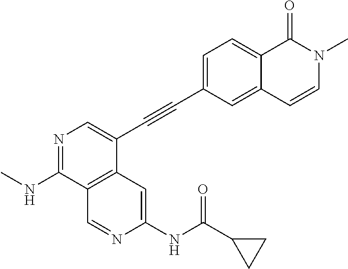 | LCMS (ESI) m/z 424.1 [M + H]⁺; ¹H NMR (400 MHz, DMSO-d₆) δ ppm 11.39 (br s, 1H), 9.58 (br s, 1H), 8.80 (s, 1H), 8.16-8.29 (m, 2H), 7.93 (s, 1H), 7.64 (br d, J = 7.0 Hz, 1H), 7.55 (d, J = 7.4 Hz, 1H), 6.61 (d, J = 7.5 Hz, 1H), 3.52 (s, 3H), 3.12 (br s, 3H), 2.08- 2.16 (m, 1H), 0.86-0.96 (m, 4H). |
| | N-(5-((2-methyl-1-oxo-1,2- | |
| | dihydroisoquinolin-6-yl)ethynyl)- | |
| | 8-(methylamino)-2,7- | |
| | naphthyridin-3- | |
| | yl)cyclopropanecarboxamide | |
| Example 1137 | 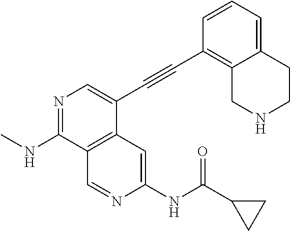 [SMILES: CNc1ncc(C#Cc2cccc3c2CNCC3)c2cc(NC(=O)C3CC3)ncc12] | LCMS (ESI) m/z 398.1 [M + H]⁺; ¹H NMR (400 MHz, DMSO-d₆) δ ppm 11.53 (s, 1H), 10.80 (br s, 1H), 9.85 (s, 1H), 9.61 (s, 1H), 8.75 (s, 1H), 8.12 (s, 1H), 7.41-7.52 (m, 1H), 7.34-7.39 (m, 3H), 4.50 (s, 2H), 3.55 (br t, J = 5.6 Hz, 2H), 3.24 (s, 3H), 3.14-3.21 (m, 2H), 1.96-2.04 (m, 1H), 1.15 (br d, J = 3.0 Hz, 2H), 0.94-1.03 (m, 2H). |
| | N-(8-(methylamino)-5-((1,2,3,4- | |
| | tetrahydroisoquinolin-8- | |
| | yl)ethynyl)-2,7-naphthyridin-3- | |
| | yl)cyclopropanecarboxamide | |

### Example 1138: Synthesis of 1-fluoro-N-(8-(methylamino)-5-((4-(2-oxopyridin-1(2H)-yl)Phenyl)ethynyl)-2,7-naphthyridin-3-yl)cyclopropanecarboxamide

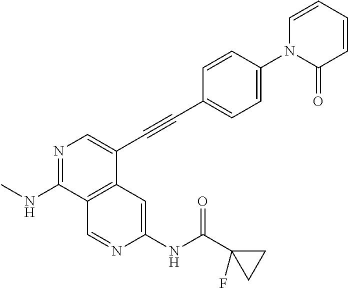 [SMILES: CNc1ncc(C#Cc2ccc(-n3ccccc3=O)cc2)c2cc(NC(=O)C3(F)CC3)ncc12]

Synthesis of: 1-fluoro-N-(8-(methylamino)-5-((4-(2-oxopyridin-1(2H)-yl)phenyl)ethynyl)-2,7-naphthyridin-3-yl)cyclopropanecarboxamide was synthesized using a similar procedure that was previously described in example XXX by using 1-(4-((6-amino-1-(methylamino)-2,7-naphthyridin-4-yl)ethynyl)phenyl)pyridin-2(1H)-one and 1-fluorocyclopropanecarboxylic acid as the starting material. LCMS (ESI) m/z 454.1 [M+H]⁺; ¹H NMR (400 MHz, DMSO-d₆) δ ppm 10.91 (br s, 1H), 9.70 (br s, 1H), 8.79 (s, 1H), 8.24 (s, 1H), 7.75 (s, 1H), 7.73 (s, 2H), 7.55-7.56 (m, 1H), 7.53 (s, 2H), 6.51 (d, J=8.00 Hz, 1H), 6.35 (br d, J=2.00 Hz, 1H), 3.14 (br d, J=4.24 Hz, 3H), 1.50-1.55 (m, 1H), 1.43-1.50 (m, 3H).

### Example 1139

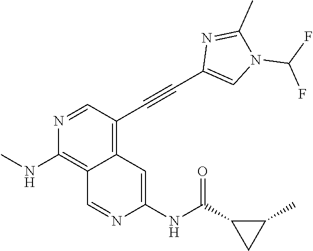 [SMILES: CNc1ncc(C#Cc2cn(C(F)F)c(C)n2)c2cc(NC(=O)[C@H]3C[C@H]3C)ncc12]

(1S,2R)—N-(5-((1-(difluoromethyl)-2-methyl-1H-imidazol-4-yl)ethynyl)-8-(methylamino)-2,7-naphthyridin-3-yl)-2-methylcyclopropanecarboxamide was synthesized using a similar procedure that was previously described in example 1010 by using 4-((1-(difluoromethyl)-2-methyl-1H-imidazol-4-yl)ethynyl)-N₁-methyl-2,7-naphthyridine-1,6-diamine and (1S,2R)-2-methylcyclopropanecarboxylic acid as the starting material. LCMS (ESI) m/z 41.1.2 [M+H]⁺; ¹H NMR (400 MHz, DMSO-d₆) δ ppm 10.97 (s, 1H), 9.35 (s, 1H), 8.52 (s, 1H), 8.33 (br d, J=4.4 Hz, 1H), 8.24 (s, 1H), 7.83 (s, 1H), 7.65-7.95 (m, 1H), 3.01 (d, J=4.4 Hz, 3H), 2.46 (s, 3H), 2.04-2.10 (m, 1H), 1.26-1.32 (m, 1H), 1.12 (d, J=6.4 Hz, 3H), 0.99 (td, J=8.0, 3.89 Hz, 1H), 0.81-0.85 (m, 1H).

### Example 1140: Synthesis of (1S,2R)—N-(5-((1-(difluoromethyl)-2-methyl-1H-imidazol-5-yl)ethynyl)-8-(methylamino)-2,7-naphthyridin-3-yl)-2-methylcyclopropanecarboxamide

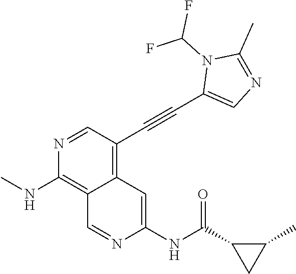

Synthesis of: (1S,2R)—N-(5-((1-(difluoromethyl)-2-methyl-1H-imidazol-5-yl)ethynyl)-8-(methylamino)-2,7-naphthyridin-3-yl)-2-methylcyclopropanecarboxamide was synthesized using a similar procedure that was previously described in example 1010 by using 4-((1-(difluoromethyl)-2-methyl-1H-imidazol-5-yl)ethynyl)-N1-methyl-2,7-naphthyridine-1,6-diamine and (1S,2R)-2-methylcyclopropanecarboxylic acid as the starting material. LCMS (ESI) m/z 411.2 [M+H]⁺. ¹H NMR (400 MHz, DMSO-d₆) δ ppm 11.02 (s, 1H), 9.36 (s, 1H), 8.53 (s, 1H), 8.42 (br d, J=4.4 Hz, 1H), 8.29 (s, 1H), 7.67-7.98 (m, 1H), 7.32 (s, 1H), 3.02 (d, J=4.4 Hz, 3H), 2.52 (s, 3H), 2.05-2.11 (m, 1H), 1.27-1.35 (m, 1H), 1.14 (d, J=6.0 Hz, 3H), 1.00 (td, J=8.0, 3.76 Hz, 1H), 0.80-0.85 (m, 1H).

### Example 1.142: Synthesis of: (1S,2R)-2-methyl-N-[8-(methylamino)-5-[2-[4-(2-methyl-5-oxo-pyrazol-1-yl)phenyl]ethynyl]-2,7-naphthyridin-3-yl]cyclopropanecarboxamide

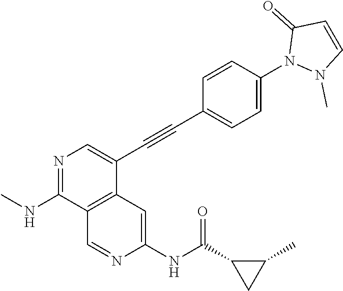 [SMILES: CNc1ncc(C#Cc2ccc(-n3c(=O)ccn3C)cc2)c2cc(NC(=O)[C@H]3C[C@H]3C)ncc12]

Synthesis of: (1S,2R)-2-methyl-N-[8-(methylamino)-5-[2-[4-(2-methyl-5-oxo-pyrazol-1-yl)phenyl]ethynyl]-2,7-naphthyridin-3-yl]cyclapropanecarboxamide was synthesized using a similar procedure that was previously described in Example 1010 by using 2-(4-((6-amino-1-(methylamino)-2,7-naphthyridin-4-yl)ethynyl)phenyl)-1-methyl-1H-pyrazol-3(2H)-one and (1S,2R)-2-methylcyclopropanecarboxylic acid as the starting material. LCMS (ESI) m/z 453.1 [M+H]⁺, ¹H NMR (400 MHz, DMSO-d₆) δ ppm 11.42 (s, 1H), 10.63 (br s, 1H), 9.78 (s, 1H), 8.85 (s, 1H), 8.11 (s, 1H), 8.05 (d, J=3.6 Hz, 1H), 7.77 (d, J=8.4 Hz, 2H), 7.49 (d, J=8.4 Hz, 2H), 5.55 (d, J=3.2 Hz, 1H), 3.21 (s, 3H), 3.18 (br. d, J=4.0 Hz, 3H), 2.10-2.18 (m, 1H), 1.31-1.43 (m, 1H), 1.16 (d, J=6.4 Hz, 3H), 1.06 (td, J=8.0, 4.0 Hz, 1H), 0.88-0.95 (m, 1H).

### Assay A: TYK2 JH2 Binding Assay

Competitive binding studies using TKY2 JH2-domain fragments were conducted by DiscoverX using the KINOMEscan (KdELECT) platform. Kinase-tagged T7 phage strains were prepared in an E. coli host derived from the BL21 strain. E. coli were grown to log-phase and infected with 17 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR. Using this data, binding constants (Kds) for test compounds were calculated in Dotmatics using the four-parameter logistic curve regression model with bottom fixed to 0 and Hill Slope fixed to −1.

Assay B: JAK1/TYK2 pSTAT5 (Tyr694/699) inhibition IC50 Cellular Assays

To evaluate the inhibition effect of compounds disclosed herein against TYK2, an IFNa-stimulated JAE1/TYK2 pSTAT5 (Tyr694/699) quantification assay was conducted in the THP-1 cell line using an AlphaLISA detection method (PerkinElmer ALSU-PST5). Compounds in DMSO were serially diluted in a 96-well plate (10 mM starting concentration, 6-fold dilutions, 8 concentrations total) prior to dispensing of 8 nL into a 384-well plate using an I.DOT non-contact liquid handler (Dispendix). Cells were collected via centrifugation at 300 ref for 5 min and resuspended in Hanks' Balanced Salt Solution (Gibco 14175-079), and 6 μl containing 2e4 cells was dispensed into each well of the 384-well plate containing test compounds or DMSO alone (final concentrations: 10000, 1666.67, 277.78, 46.30, 7.72, 1.29, 0.21 and 0.04 nM). Cells were then incubated for 1 h at 37° C. with 5% CO₂ prior to the addition of 2 μL IFNa (100 ng/mL; PBL Assay Science 11175-1). Following an additional 30 min incubation at 37° C. with 5% CO₂, 2 μL of 5× lysis buffer was added to each well using the I.DOT (name/product ID needed). Plates were sealed and incubated at RT for 10 min on a plate shaker set to 400 RPM. Cell lysates were then frozen at −80° C. before subsequent detection of pSTAT5 by AlphaLISA. At the time of the detection assay, cell lysates were thawed for 5 min at room temperature and then on a plate shaker set to 400 RPM for an additional 10 min. Following the manufacturer recommended protocol (Perkin Elmer), 5 μL of acceptor bead master mix was added to each well; plates were then resealed and incubated for 2 min at RT on a plate shaker (400 rpm) prior to an additional 2-h incubation without agitation (protected from light). Donor bead master mix, 5 uL per well, was then added to each well before 2 min of shaking (RT, 400 rpm) and 2-h incubation (RT, no shaking, protected from light). Levels of pSTAT5 protein were then measured as fluorescent signal at 615 nm with an EnVision multimode plate reader (Perkin Elmer). IC50 values were calculated in Dotmatics using the four-parameter logistic curve regression model with bottom fixed to 0 and Hill Slope fixed to −1.

Compounds with TYK2 JH2 Kd (as measured in Assay A) equal to or less than 100 nM are designated as A, between 100 nM and 1,000 nM as B, between 1,000 nM and 10,000 as C, larger than 10,000 nM: as D. Compounds with IFNa IC50 in IFNa, cellular assay (as measured in Assay B) equal to or less than 500 nM are designated as A, larger than 500 nM as B.

| | | Cellular |
|---|---|---|
| | Biochemical | THP1_IFNa- |
| Example | TYK2_JH2_Kd | pSTATS_IC50 |
| 1 | A | B |
| 2 | B | B |
| 3 | A | B |
| 4 | A | B |
| 5 | B | B |
| 6 | A | B |
| 7 | B | B |
| 8 | A | B |
| 9 | A | B |
| 10 | A | B |
| 11 | B | B |
| 12 | A | B |
| 13 | A | B |
| 14 | A | B |
| 15 | A | B |
| 16 | A | B |
| 17 | A | B |
| 18 | A | B |
| 19 | A | B |
| 20 | A | B |
| 21 | A | B |
| 22 | A | B |
| 23 | A | B |
| 24 | A | B |
| 25 | A | B |
| 26 | A | B |
| 27 | A | B |
| 28 | A | B |
| 29 | A | B |
| 30 | A | B |
| 31 | A | B |
| 32 | A | A |
| 33 | A | A |
| 34 | B | B |
| 35 | A | B |
| 36 | A | B |
| 37 | A | B |
| 38 | A | B |
| 39 | C | B |
| 40 | A | B |
| 41 | B | B |
| 42 | B | B |
| 43 | A | B |
| 44 | A | B |
| 45 | A | B |
| 46 | A | B |
| 47 | B | B |
| 48 | C | B |
| 49 | A | B |
| 50 | A | B |
| 51 | C | B |
| 52 | A | B |
| 53 | A | B |
| 54 | A | B |
| 55 | A | B |
| 56 | A | B |
| 57 | A | B |
| 58 | B | nd |
| 59 | C | nd |
| 60 | B | nd |
| 61 | C | nd |
| 62 | A | nd |
| 63 | A | B |
| 64 | A | A |
| 65 | A | B |
| 66 | A | B |
| 67 | A | B |
| 68 | A | B |
| 69 | A | B |
| 70 | A | B |
| 71 | A | B |
| 72 | A | B |
| 73 | A | B |
| 74 | A | B |
| 75 | A | B |
| 76 | A | B |
| 77 | A | B |
| 78 | A | B |
| 79 | A | B |
| 80 | A | B |
| 81 | A | B |
| 82 | A | B |
| 83 | A | B |
| 84 | A | B |
| 85 | B | B |
| 86 | A | B |
| 87 | A | B |
| 88 | A | B |
| 89 | B | B |
| 90 | A | B |
| 91 | B | B |
| 92 | A | B |
| 93 | C | B |
| 94 | B | B |
| 95 | B | B |
| 96 | A | B |
| 97 | B | nd |
| 98 | C | B |
| 99 | C | nd |
| 100 | C | nd |
| 101 | A | B |
| 102 | B | nd |
| 103 | A | B |
| 104 | A | B |
| 105 | A | B |
| 106 | A | B |
| 107 | A | nd |
| 108 | A | B |
| 109 | A | B |
| 110 | A | B |
| 111 | A | B |
| 112 | A | B |
| 113 | A | B |
| 114 | A | A |
| 115 | A | B |
| 116 | A | B |
| 117 | A | B |
| 118 | A | A |
| 119 | A | B |
| 120 | B | B |
| 121 | C | nd |
| 122 | B | nd |
| 123 | A | nd |
| 124 | A | B |
| 125 | A | B |
| 126 | A | B |
| 127 | A | B |
| 128 | A | B |
| 129 | A | B |
| 130 | A | B |
| 131 | A | B |
| 132 | A | B |
| 133 | A | B |
| 134 | B | B |
| 135 | A | A |
| 136 | B | B |
| 137 | A | A |
| 138 | A | B |
| 139 | A | B |
| 140 | A | B |
| 141 | A | B |
| 142 | A | A |
| 143 | A | A |
| 144 | A | B |
| 145 | A | A |
| 146 | A | nd |
| 147 | A | A |
| 148 | A | A |
| 149 | A | B |
| 150 | A | B |
| 151 | A | B |
| 152 | A | B |
| 153A | A | B |
| 153B | A | B |
| 154 | A | B |
| 155 | A | A |
| 156 | A | B |
| 157 | A | A |
| 159 | B | nd |
| 160 | A | B |
| 161 | A | B |
| 162 | B | B |
| 163 | B | B |
| 164 | B | B |
| 165 | A | B |
| 166 | A | B |
| 167 | A | A |
| 168 | A | B |
| 169 | A | B |
| 170 | A | A |
| 171 | A | A |
| 172 | A | B |
| 173 | A | A |
| 174 | nd | A |
| 175 | B | B |
| 176 | B | B |
| 177 | A | B |
| 178 | A | B |
| 179 | A | A |
| 180 | A | A |
| 181 | A | A |
| 182 | A | Nd |
| 183 | Nd | Nd |
| 184 | A | A |
| 185 | A | Nd |
| 186 | Nd | Nd |
| 187 | Nd | Nd |
| 188 | A | A |
| 189 | A | Nd |
| 190 | A | Nd |
| 191 | A | Nd |
| 192 | A | Nd |
| 193 | A | Nd |
| 194 | A | Nd |

Nd: not determined

| | Biochemical TYK2_Dis- | Cellular_THP1_IFNa- |
|---|---|---|
| Example | coverX_JH2_BND_Kd | pSTATS_TYK2 |
| 195 | A | A |
| 196 | A | A |
| 197 | A | A |
| 198 | A | A |
| 199 | A | A |
| 200 | A | A |
| 201 | A | A |
| 202 | A | A |
| 203 | A | A |
| 204 | A | A |
| 205 | A | A |
| 206 | A | A |
| 207 | A | A |
| 208 | A | A |
| 209 | A | A |
| 210 | A | A |
| 211 | A | A |
| 212 | A | A |
| 213 | A | A |
| 214 | A | B |
| 215 | A | A |
| 216 | A | A |
| 217 | A | A |
| 218 | A | A |
| 219 | A | A |
| 220 | A | B |
| 221 | A | A |
| 222 | A | A |
| 223 | A | A |
| 224 | A | A |
| 225 | A | A |
| 226 | A | A |
| 227 | A | A |
| 228 | A | A |
| 229 | A | A |
| 230 | A | A |
| 231 | A | A |
| 232 | A | A |
| 233 | A | A |
| 234 | A | A |
| 235 | A | A |
| 236 | A | A |
| 237 | A | A |
| 238 | A | A |
| 239 | A | A |
| 240 | A | A |
| 241 | A | A |
| 242 | A | A |
| 243 | A | A |
| 244 | A | A |
| 245 | A | A |
| 246 | A | A |
| 247 | A | A |
| 248 | A | A |
| 249 | A | A |
| 250 | A | A |
| 251 | A | A |
| 252 | A | A |
| 253 | A | A |
| 254 | A | A |
| 255 | A | A |
| 256 | A | A |
| 257 | A | A |
| 258 | A | A |
| 259 | A | A |
| 260 | A | A |
| 261 | A | A |
| 262 | A | A |
| 263 | A | B |
| 264 | A | A |
| 265 | A | A |
| 266 | A | A |
| 267 | A | A |
| 268 | A | A |
| 269 | A | A |
| 270 | A | A |
| 271 | A | A |
| 272 | A | A |
| 273 | A | A |
| 274 | A | A |
| 275 | A | A |
| 276 | A | A |
| 277 | A | A |
| 278 | A | A |
| 279 | A | A |
| 280 | A | A |
| 281 | A | A |
| 282 | A | A |
| 283 | A | A |
| 284 | A | A |
| 285 | A | A |
| 286 | A | A |
| 287 | A | A |
| 288 | A | A |
| 289 | A | A |
| 290 | A | A |
| 291 | A | A |
| 292 | A | A |
| 293 | A | A |
| 294 | A | A |
| 295 | A | A |
| 296 | A | A |
| 297 | A | A |
| 298 | A | A |
| 299 | A | A |
| 300 | A | A |
| 301 | A | A |
| 302 | A | A |
| 303 | A | A |
| 304 | A | A |
| 305 | A | A |
| 306 | A | A |
| 307 | A | A |
| 308 | A | A |
| 309 | A | A |
| 310 | A | A |
| 311 | A | A |
| 312 | A | A |
| 313 | A | A |
| 314 | A | A |
| 315 | A | A |
| 316 | A | A |
| 317 | A | A |
| 318 | A | A |
| 319 | A | A |
| 320 | A | A |
| 321 | A | A |
| 322 | A | A |
| 323 | A | A |
| 324 | A | A |
| 325 | A | A |
| 326 | A | A |
| 327 | A | A |
| 328 | A | A |
| 329 | A | A |
| 330 | A | A |
| 331 | A | A |
| 332 | A | A |
| 333 | A | A |
| 334 | A | A |
| 335 | A | A |
| 336 | A | A |
| 337 | A | B |
| 338 | A | A |
| 339 | A | A |
| 340 | A | A |
| 341 | A | A |
| 342 | A | A |
| 343 | A | A |
| 344 | A | A |
| 345 | A | A |
| 346 | A | A |
| 347 | A | A |
| 348 | A | A |
| 349 | A | A |
| 350 | A | A |
| 351 | A | A |
| 352 | A | A |
| 353 | A | A |
| 354 | A | A |
| 355 | A | A |
| 356 | A | A |
| 357 | A | B |
| 358 | A | A |
| 359 | A | A |
| 360 | A | A |
| 361 | A | A |
| 362 | A | A |
| 363 | A | A |
| 364 | A | A |
| 365 | A | A |
| 366 | A | A |
| 367 | A | A |
| 368 | A | A |
| 369 | A | A |
| 370 | A | A |
| 371 | A | B |
| 372 | A | A |
| 372 | A | A |
| 374 | A | B |
| 375 | A | A |
| 376 | A | A |
| 377 | A | A |
| 378 | A | A |
| 379 | A | A |
| 380 | A | A |
| 381 | A | B |
| 382 | A | A |
| 383 | A | A |
| 384 | A | A |
| 385 | A | A |
| 386 | A | A |
| 387 | A | A |
| 388 | A | A |
| 389 | A | A |
| 390 | A | A |
| 391 | A | A |
| 392 | A | ND |
| 393 | A | A |
| 394 | A | A |
| 399 | A | A |
| 396 | A | A |
| 397 | A | A |
| 398 | A | B |
| 399 | A | A |
| 400 | A | A |
| 401 | A | A |
| 402 | A | B |
| 403 | A | B |
| 404 | A | A |
| 405 | A | A |
| 406 | A | A |
| 407 | A | A |
| 408 | A | A |
| 409 | A | A |
| 410 | A | A |
| 411 | A | A |
| 412 | A | A |
| 413 | A | A |
| 414 | A | A |
| 415 | A | A |
| 416 | A | A |
| 417 | A | A |
| 418 | A | A |
| 419 | A | A |
| 420 | A | A |
| 421 | A | A |
| 422 | A | A |
| 423 | A | A |
| 424 | A | A |
| 425 | A | A |
| 426 | A | A |
| 427 | A | A |
| 428 | A | A |
| 429 | A | B |
| 430 | B | ND |
| 431 | A | A |
| 432 | A | A |
| 433 | A | A |
| 434 | A | A |
| 435 | A | A |
| 436 | A | A |
| 437 | A | A |
| 438 | A | A |
| 439 | A | A |
| 440 | A | A |
| 441 | A | A |
| 442 | A | A |
| 443 | A | A |
| 444 | A | A |
| 445 | A | A |
| 446 | A | B |
| 447 | A | A |
| 448 | A | A |
| 449 | A | A |
| 450 | A | A |
| 451 | A | A |
| 452 | A | A |
| 453 | A | A |
| 454 | A | A |
| 455 | A | A |
| 456 | A | A |
| 457 | A | A |
| 458 | A | A |
| 459 | A | A |
| 460 | A | A |
| 461 | ND | A |
| 462 | A | A |
| 463 | A | A |
| 464 | A | A |
| 465 | ND | A |
| 466 | ND | A |
| 467 | A | A |
| 468 | A | A |
| 469 | A | A |
| 470 | A | A |
| 471 | A | A |
| 472 | A | A |
| 473 | A | A |
| 474 | A | A |
| 475 | A | A |
| 476 | A | A |
| 477 | A | A |
| 478 | A | A |
| 479 | A | A |
| 480 | A | A |
| 481 | A | A |
| 482 | A | A |
| 483 | A | A |
| 484 | A | A |
| 485 | A | A |
| 486 | A | A |
| 487 | A | A |
| 488 | A | A |
| 489 | A | A |
| 490 | A | A |
| 491 | A | A |
| 492 | A | A |
| 493 | A | A |
| 493A | A | A |
| 494 | A | A |
| 495 | A | A |
| 496 | A | A |
| 497 | A | A |
| 498 | A | A |
| 499 | A | A |
| 500 | A | A |
| 501 | A | A |
| 502 | A | A |
| 503 | A | A |
| 504 | A | A |
| 505 | A | A |
| 506 | A | A |
| 507 | A | A |
| 508 | A | A |
| 509 | A | A |
| 510 | A | A |
| 511 | A | A |
| 512 | A | A |
| 513 | A | A |
| 514 | A | A |
| 515 | A | A |
| 516 | A | A |
| 517 | A | A |
| 518 | A | A |
| 519 | A | B |
| 520 | A | A |
| 521 | A | A |
| 522 | A | A |
| 523 | A | A |
| 524 | A | A |
| 525 | A | A |
| 526 | A | A |
| 527 | A | A |
| 528 | A | A |
| 529 | A | A |
| 530 | A | A |
| 531 | A | A |
| 532 | A | A |
| 533 | A | B |
| 534 | A | A |
| 535 | A | A |
| 536 | A | A |
| 537 | A | A |
| 538 | A | A |
| 539 | A | A |
| 540 | A | A |
| 541 | A | A |
| 542 | A | A |
| 543 | A | A |
| 544 | A | A |
| 545 | A | A |
| 546 | A | A |
| 547 | A | A |
| 548 | A | A |
| 549 | A | A |
| 550 | A | A |
| 551 | A | A |
| 552 | A | A |
| 553 | A | B |
| 554 | A | A |
| 555 | A | A |
| 556 | A | A |
| 557 | A | A |
| 558 | A | A |
| 559 | A | A |
| 560 | A | A |
| 561 | A | A |
| 562 | A | A |
| 563 | A | A |
| 564 | A | A |
| 565 | A | A |
| 566 | A | A |
| 567 | A | A |
| 568 | A | A |
| 569 | A | A |
| 570 | A | A |
| 571 | A | A |
| 572 | A | A |
| 573 | A | A |
| 574 | A | A |
| 576 | A | A |
| 577 | A | B |
| 578 | A | A |
| 579 | A | A |
| 580 | A | A |
| 581 | A | A |
| 582 | A | A |
| 583 | A | A |
| 584 | A | A |
| 585 | A | A |
| 586 | A | A |
| 587 | A | A |
| 588 | A | A |
| 589 | A | A |
| 590 | A | A |
| 591 | A | A |
| 592 | A | B |
| 593 | A | A |
| 594 | A | A |
| 595 | A | A |
| 596 | A | A |
| 597 | A | A |
| 598 | A | A |
| 599 | A | A |
| 600 | A | A |
| 601 | A | A |
| 602 | A | A |
| 607 | A | A |
| 608 | A | A |
| 609 | A | A |
| 610 | A | A |
| 611 | A | A |
| 612 | A | A |
| 613 | A | A |
| 614 | A | A |
| 615 | A | A |
| 616 | A | A |
| 617 | A | A |
| 618 | A | A |
| 619 | A | A |
| 620 | A | A |
| 621 | A | A |
| 622 | A | A |
| 623 | A | A |
| 624 | A | A |
| 625 | A | A |
| 626 | A | A |
| 627 | A | A |
| 628 | A | A |
| 629 | A | A |
| 630 | A | A |
| 631 | A | A |
| 632 | A | A |
| 633 | A | A |
| 634 | A | A |
| 635 | A | A |
| 636 | A | B |
| 637 | A | A |
| 638 | A | A |
| 639 | A | A |
| 640 | A | A |
| 641 | A | A |
| 642 | A | A |
| 643 | A | A |
| 644 | A | A |
| 645 | A | A |
| 646 | A | A |
| 647 | A | A |
| 648 | A | A |
| 649 | A | A |
| 650 | A | A |
| 651 | A | A |
| 652 | A | A |
| 653 | A | A |
| 654 | A | A |
| 655 | A | A |
| 656 | A | A |
| 657 | A | A |
| 658 | A | A |
| 659 | A | A |
| 660 | A | A |
| 661 | A | A |
| 662 | A | A |
| 663 | A | A |
| 664 | A | A |
| 665 | A | A |
| 666 | A | A |
| 667 | A | A |
| 668 | A | A |
| 669 | A | A |
| 670 | A | A |
| 671 | A | A |
| 672 | A | A |
| 673 | A | A |
| 674 | A | A |
| 675 | A | A |
| 676 | A | A |
| 677 | A | A |
| 678 | A | A |
| 679 | A | A |
| 680 | A | A |
| 681 | A | A |
| 682 | A | A |
| 683 | A | A |
| 684 | A | A |
| 685 | A | A |
| 686 | A | A |
| 687 | A | A |
| 688 | A | A |
| 689 | A | A |
| 690 | A | A |
| 691 | A | A |
| 692 | A | A |
| 693 | A | A |
| 694 | A | A |
| 695 | A | A |
| 696 | A | A |
| 697 | A | A |
| 698 | A | A |
| 699 | A | A |
| 700 | A | A |
| 701 | A | A |
| 702 | A | A |
| 703 | A | A |
| 704 | A | A |
| 705 | A | A |
| 706 | A | A |
| 707 | A | A |
| 708 | A | A |
| 709 | A | A |
| 710 | A | A |
| 711 | A | A |
| 712 | A | A |
| 713 | A | A |
| 714 | A | A |
| 715 | A | A |
| 716 | A | A |
| 717 | A | A |
| 718 | A | A |
| 719 | A | A |
| 720 | A | A |
| 721 | A | A |
| 722 | A | A |
| 723 | A | A |
| 724 | A | A |
| 725 | A | A |
| 726 | A | A |
| 727 | A | A |
| 728 | A | A |
| 729 | A | A |
| 730 | A | B |
| 731 | A | B |
| 732 | A | A |
| 733 | A | A |
| 734 | A | A |
| 735 | A | A |
| 736 | A | A |
| 737 | A | A |
| 738 | A | A |
| 739 | A | A |
| 740 | A | A |
| 741 | A | A |
| 742 | A | A |
| 743 | A | A |
| 744 | A | A |
| 745 | A | A |
| 746 | A | A |
| 747 | A | A |
| 748 | A | A |
| 749 | A | A |
| 750 | A | A |
| 751 | A | A |
| 752 | A | A |
| 753 | A | A |
| 754 | A | A |
| 755 | A | A |
| 756 | A | A |
| 757 | A | A |
| 758 | A | A |
| 759 | A | B |
| 760 | A | A |
| 761 | A | A |
| 762 | A | A |
| 763 | A | A |
| 764 | A | A |
| 765 | A | A |
| 766 | A | A |
| 767 | A | A |
| 768 | A | A |
| 769 | A | A |
| 770 | A | A |
| 771 | A | A |
| 772 | A | A |
| 773 | A | A |
| 774 | A | A |
| 775 | A | A |
| 776 | A | A |
| 777 | A | A |
| 778 | A | A |
| 779 | A | A |
| 780 | A | B |
| 781 | A | B |
| 782 | A | A |
| 783 | A | A |
| 784 | A | A |
| 785 | A | B |
| 786 | A | A |
| 787 | A | A |
| 788 | B | B |
| 789 | A | A |
| 790 | A | A |
| 791 | A | A |
| 792 | A | A |
| 793 | A | B |
| 794 | A | A |
| 795 | A | A |
| 796 | A | A |
| 797 | ND | A |
| 798 | A | A |
| 799 | A | A |
| 800 | A | A |
| 801 | A | A |
| 802 | ND | A |
| 803 | ND | A |
| 804 | A | A |
| 805 | A | A |
| 806 | A | A |
| 807 | A | A |
| 808 | A | A |
| 809 | A | A |
| 810 | ND | A |
| 811 | A | A |
| 812 | A | A |
| 813 | A | A |
| 814 | ND | A |
| 815 | A | A |
| 816 | A | A |
| 817 | A | A |
| 818 | A | A |
| 819 | A | A |
| 820 | A | A |
| 821 | A | A |
| 822 | A | A |
| 823 | A | A |
| 824 | B | B |
| 825 | A | B |
| 826 | A | B |
| 827 | A | B |
| 828 | A | B |
| 829 | A | A |
| 830 | A | A |
| 831 | A | B |
| 832 | A | B |
| 833 | A | B |
| 834 | A | B |
| 835 | A | B |
| 835A | A | B |
| 836 | A | B |
| 837 | A | ND |
| 838 | A | ND |
| 839 | A | A |
| 840 | B | ND |
| 841 | A | B |
| 842 | A | B |
| 843 | A | A |
| 844 | A | B |
| 845 | A | B |
| 846 | A | B |
| 847 | A | A |
| 848 | A | B |
| 849 | A | B |
| 850 | A | A |
| 851 | A | A |
| 852 | A | B |
| 853 | A | B |
| 854 | B | ND |
| 855 | A | B |
| 856 | B | ND |
| 857 | A | A |
| 858 | A | A |
| 859 | A | A |
| 860 | A | B |
| 861 | A | A |
| 862 | A | A |
| 863 | A | A |
| 864 | A | A |
| 865 | A | A |
| 866 | A | A |
| 867 | A | A |
| 868 | A | A |
| 869 | A | B |
| 870 | A | A |
| 871 | A | A |
| 872 | A | A |
| 873 | A | A |
| 874 | A | A |
| 875 | A | A |
| 876 | A | A |
| 877 | A | A |
| 878 | A | B |
| 879 | A | A |
| 880 | A | A |
| 881 | A | B |
| 882 | A | A |
| 883 | A | A |
| 884 | A | A |
| 885 | A | A |
| 886 | A | A |
| 887 | A | A |
| 888 | A | A |
| 889 | A | B |
| 890 | A | A |
| 891 | ND | A |
| 892 | ND | A |
| 893 | A | A |
| 894 | A | A |
| 895 | A | A |
| 896 | A | A |
| 897 | A | A |
| 898 | A | A |
| 899 | A | A |
| 900 | A | A |
| 901 | A | A |
| 902 | A | A |
| 903 | A | A |
| 904 | A | A |
| 905 | ND | A |
| 906 | ND | A |
| 907 | ND | A |
| 908 | ND | A |
| 909 | ND | A |
| 910 | ND | A |
| 911 | ND | A |
| 912 | ND | A |
| 913 | A | A |
| 914 | A | A |
| 915 | A | B |
| 916 | A | A |
| 917 | A | A |
| 918 | A | A |
| 919 | A | A |
| 920 | A | A |
| 921 | A | A |
| 922 | A | A |
| 923 | A | A |
| 924 | A | A |
| 925 | A | A |
| 926 | A | A |
| 927 | A | A |
| 928 | A | A |
| 929 | A | A |
| 930 | A | A |
| 931 | A | A |
| 932 | A | A |
| 933 | A | A |
| 934 | A | A |
| 935 | A | A |
| 936 | A | A |
| 937 | A | A |
| 938 | A | A |
| 939 | A | A |
| 940 | A | A |
| 941 | A | A |
| 942 | A | A |
| 943 | A | A |
| 944 | A | A |
| 945 | A | A |
| 946 | A | A |
| 947 | A | A |
| 948 | A | A |
| 949 | A | A |
| 950 | A | A |
| 951 | A | A |
| 952 | A | A |
| 953 | A | A |
| 954 | A | A |
| 955 | A | A |
| 956 | A | A |
| 957 | A | A |
| 958 | A | A |
| 959 | A | A |
| 960 | A | A |
| 961 | A | A |
| 962 | A | A |
| 963 | A | A |
| 964 | A | A |
| 965 | A | A |
| 966 | A | A |
| 967 | A | A |
| 968 | A | A |
| 969 | A | A |
| 970 | A | A |
| 971 | A | A |
| 972 | A | A |
| 973 | A | A |
| 974 | A | A |
| 975 | A | A |
| 976 | A | A |
| 977 | A | A |
| 978 | A | A |
| 979 | A | A |
| 980 | A | A |
| 981 | A | A |
| 982 | A | A |
| 983 | A | A |
| 984 | A | A |
| 985 | A | A |
| 986 | A | A |
| 987 | A | A |
| 988 | A | A |
| 989 | A | A |
| 990 | A | A |
| 991 | A | A |
| 992 | A | A |
| 993 | A | A |
| 994 | A | A |
| 995 | A | A |
| 996 | A | A |
| 997 | A | A |
| 998 | A | A |
| 999 | A | A |
| 1000 | A | A |
| 1001 | A | A |
| 1002 | A | A |
| 1003 | A | A |
| 1004 | A | A |
| 1005 | A | A |
| 1006 | A | A |
| 1007 | A | A |
| 1008 | A | A |
| 1009 | A | A |
| 1010 | A | A |
| 1011 | A | A |
| 1012 | A | A |
| 1013 | A | A |
| 1014 | A | A |
| 1015 | A | A |
| 1016 | A | A |
| 1017 | A | A |
| 1018 | A | A |
| 1019 | A | A |
| 1020 | A | A |
| 1021 | A | A |
| 1022 | A | A |
| 1023 | A | A |
| 1024 | A | A |
| 1025 | A | B |
| 1026 | A | A |
| 1027 | A | A |
| 1028 | A | A |
| 1029 | A | A |
| 1030 | A | A |
| 1031 | A | A |
| 1032 | A | A |
| 1033 | A | A |
| 1034 | A | A |
| 1035 | A | A |
| 1036 | A | A |
| 1037 | A | A |
| 1038 | A | A |
| 1039 | A | A |
| 1040 | A | A |
| 1041 | A | A |
| 1042 | A | A |
| 1043 | A | A |
| 1044 | A | A |
| 1045 | A | A |
| 1046 | A | A |
| 1047 | A | A |
| 1048 | A | A |
| 1049 | A | A |
| 1050 | A | A |
| 1051 | A | A |
| 1052 | A | A |
| 1053 | A | A |
| 1054 | A | A |
| 1055 | A | A |
| 1056 | A | A |
| 1057 | A | A |
| 1058 | A | A |
| 1059 | A | A |
| 1060 | A | A |
| 1061 | A | A |
| 1062 | A | A |
| 1063 | A | A |
| 1064 | A | A |
| 1065 | A | A |
| 1066 | A | A |
| 1067 | A | A |
| 1068 | A | A |
| 1069 | A | A |
| 1070 | A | A |
| 1071 | A | A |
| 1072 | A | A |
| 1073 | A | A |
| 1074 | A | A |
| 1075 | A | A |
| 1076 | A | A |
| 1077 | A | A |
| 1078 | A | A |
| 1079 | A | A |
| 1080 | A | A |
| 1081 | A | A |
| 1082 | A | A |
| 1083 | A | B |
| 1084 | A | B |
| 1085 | A | A |
| 1086 | A | A |
| 1087 | A | B |
| 1088 | A | A |
| 1089 | A | A |
| 1090 | A | B |
| 1091 | A | A |
| 1092 | A | A |
| 1093 | A | A |
| 1094 | A | A |
| 1095 | A | A |
| 1096 | A | A |
| 1097 | A | A |
| 1098 | A | A |
| 1099 | A | A |
| 1100 | A | A |
| 1101 | A | A |
| 1102 | A | A |
| 1103 | A | A |
| 1104 | ND | A |
| 1135 | ND | A |
| 1136 | ND | A |
| 1137 | ND | B |
| 1138 | A | A |
| 1139 | ND | A |
| 1140 | ND | A |
| 1141 | A | A |
| 1142 | A | A |

### INCORPORATION BY REFERENCE

Reference and citations to other documents, such as patents, patent applications, patent publications, journals, books, papers, web contents, have been made through out this disclosure. All such documents are hereby incorporated by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A compound of formula (I): 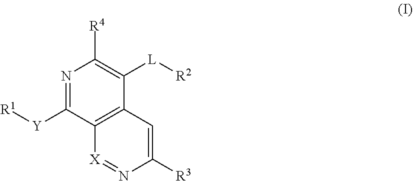
and pharmaceutically acceptable salts thereof,
wherein:
X is CH or N;
Y is CH₂, S, or NH;
L is a single bond, double bond, triple bond substituted or unsubstituted alkyl, heteroalkyl, alkoxy, heteroalkoxy, cycloalkyl, heterocycloalkyl, haloalkyl, halocycloalkyl, aryl, heteroaryl, —C(O)NH—, —NHC(O)—, O, NH, or S;
R¹ is alkyl, cycloalkyl, haloalkyl, halocycloalkyl, aryl, heteroaryl, methyl, CD₃, or H;
R² is H, halo, alkyl, branched alkyl, alkenyl, alkynyl, haloalkyl, alkoxy, cycloalkoxy, haloalkoxy, nitro, cyano, or a 5 or 6 membered substituted or unsubstituted aryl, or monocyclic or bicyclic heteroaryl ring optionally containing one or more heteroatoms independently selected from O, S, and N,
wherein the substitutions on the said 5 or 6 membered aryl or heteroaryl rings are: H, halo, alkyl, branched alkyl, alkenyl, alkynyl, haloalkyl, alkoxy, cycloalkoxy, haloalkoxy, thioalkyl, nitro, cyano, —CH₂-cycloalkyl, —CF₂-cycloalky, —CH(CH₃)-cycloalkyl, —CH₂-aryl, —CF₃, —CF₂-aryl, —CH(CH₃)-aryl, C(═O)-alkyl, —C(═O)cycloalkyl, —C(═O)—NH-alkyl, —C(═O)NH₂, hydroxy, —COOH (and ester thereof), sulfonyl, alkylsulfonyl, arylsulfonyl, sulfonamide, amino, 3-6 membered cycloalkyl or heterocycloalkyl, 3-6 membered aryl or heteroaryl, any of which may have one or more substituents;
R³ is H, halo, alkyl, branched alkyl, alkenyl, alkynyl, cycloalkyl, haloalkyl, alkoxy, cycloalkoxy, haloalkoxy, nitro, cyano, aminoalkyl, aminocycloalkyl, aminoheterocycloalkyl, —NH-aryl, —NH-heteroaryl, —NH-phenyl, —NH₂, —NH—CH—CF₃, substituted or unsubstituted C(═O)cycloalkyl, substituted or unsubstituted —NH—C(═O)cycloalkyl, —NH—C(═O)alkyl, substituted or unsubstituted —NH—C(═O)cycloalkyl, substituted or unsubstituted aminoalkylaryl;
R⁴ is selected from a group consisting of: H, halo, alkyl, branched alkyl, alkenyl, alkynyl, cycloalkyl, haloalkyl alkoxy, cycloalkoxy, haloalkoxy, nitro, cyano, and alkylhydroxyl.

2. The compound of claim 1, wherein Y is NH.

3. The compound of claim 1, wherein R₁ is methyl or ethyl.

4. The compound of claim 1, wherein L is a single bond.

5. The compound of claim 1, wherein X is CH.

6. The compound of claim 1, wherein X is N.

7. The compound of claim 1, wherein R³ is: 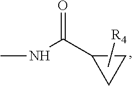
wherein R₄ is halo, alkyl, branched alkyl, alkenyl, alkynyl, cycloalkyl, spirocycloalkyl, haloalkyl, alkoxy, cycloalkoxy, haloalkoxy, nitro, or cyano.

8. The compound of claim 1, wherein R³ is:

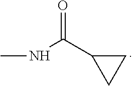

9. The compound of claim 1, wherein R² is: 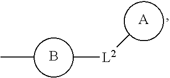
wherein L² is substituted or unsubstituted alkyl, heteroalkyl, alkoxy, heteroalkoxy, cycloalkyl, heterocycloalkyl, haloalkyl, halocycloalkyl, aryl, heteroaryl, or a bond linking the groups;
A or B are independently 5 or 6 membered substituted or unsubstituted aryl or heteroaryl ring optionally containing one or more heteroatoms independently selected from O, S, and N, wherein the substitutions on the said 5 or 6 membered aryl or heteroaryl ring are: H, halo, alkyl, branched alkyl, alkenyl, alkynyl, haloalkyl, alkoxy, cycloalkoxy, haloalkoxy, nitro, cyano, —CH₂-cycloalkyl, —CF₂-cycloalky, —CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —C(═O)NH₂, hydroxy, —COOH (and ester thereof), alkylsulfonyl, aryl sulfonyl, sulfonamide, amino, 3-6 membered cycloalkyl or heterocycloalkyl, 3-6 membered aryl or heteroaryl, any of which may have one or more substituents.

10. The compound of claim 1, wherein R₂ is phenyl.

11. The compound of claim 1, wherein R₂ is: 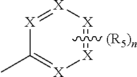
wherein each X is independently N or CH;
R⁵ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR₆, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or un substituted spirobicycloheteroalkyl, substituted or unsubstituted aryl, and substituted or unsubstituted heteroaryl;
and n is 0, 1, 2, or 3.

12. The compound of claim 1, wherein R² is: 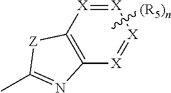
wherein Z is O or S, each X is independently N or CH;
R₅ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, substituted or unsubstituted heteroaryl;
and n is 0,1, 2 or 3.

13. The compound of claim 1, R² is: 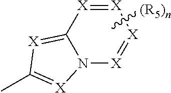
wherein each X is independently N or CH; R is selected from a group consisting of halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, substituted or unsubstituted heteroaryl;
and n is 0,1, 2 or 3.

14. The compound of claim 1, wherein L-R² is: 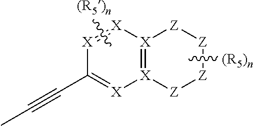
wherein each X is independently N or CH;
Z is independently O or NR⁶;
R⁵ and R^{5′} is independently halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂), S(O)R⁶, —(CH₂)ₙOR⁶, —P(O) R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, or substituted or unsubstituted heteroaryl;
and n is 0, 1, 2 or 3.

15. The compound of claim 1, wherein L-le is: 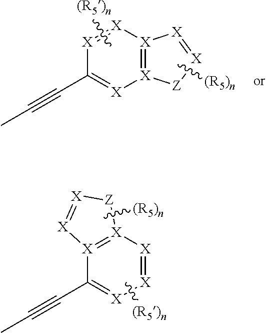
wherein Z is —N or O,
each X is independently N or CH;
R₅ and R₅′ is independently halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, (CH₂)ₙOR⁶, —P(O) R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, or substituted or unsubstituted heteroaryl; and
n is 0, 1, 2, or 3.

16. The compound of claim 1, wherein L-R² is: 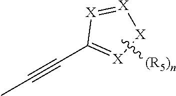
wherein R₅ is selected from a group consisting of: H, halogen, hydroxyl, —CN, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O) R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, and substituted or unsubstituted heteroaryl; and
n is 0, 1, 2, or 3.

17. The compound of claim 1; wherein the compound is selected from the group consisting of:

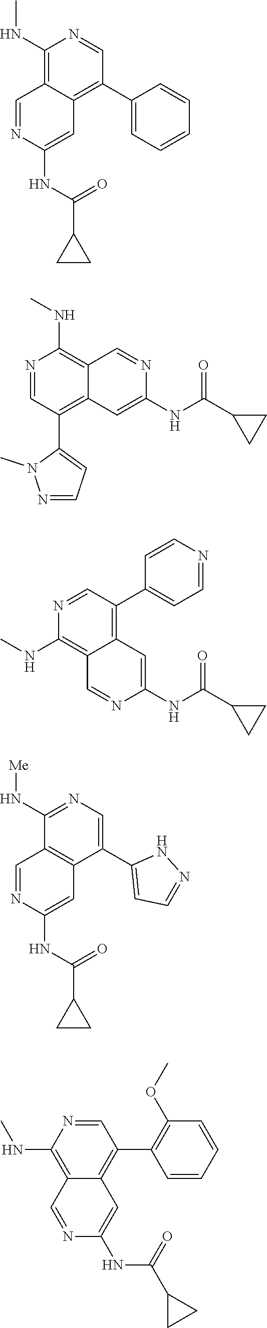

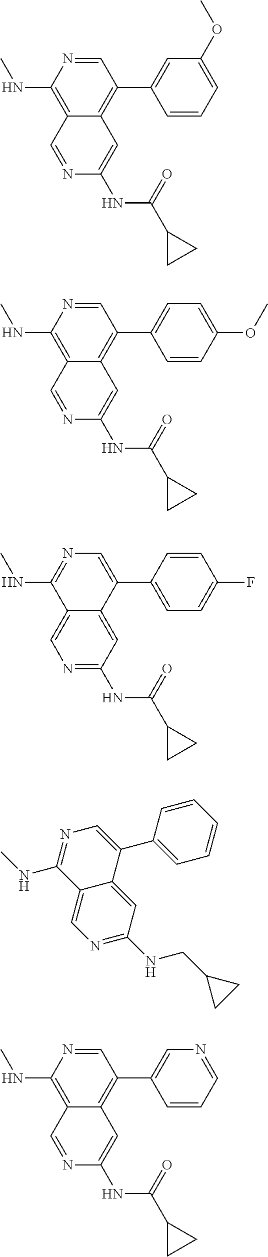

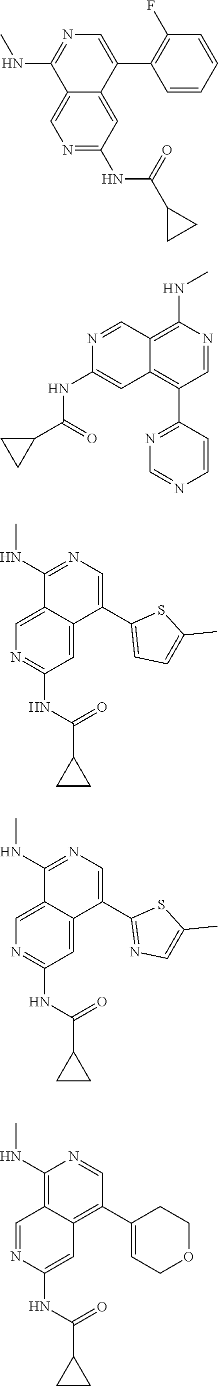 [SMILES: CNc1ncc(-c2ccccc2F)c2cc(NC(=O)C3CC3)ncc12 ; CNc1ncc(-c2ccncn2)c2cc(NC(=O)C3CC3)ncc12 ; CNc1ncc(-c2ccc(C)s2)c2cc(NC(=O)C3CC3)ncc12 ; CNc1ncc(-c2ncc(C)s2)c2cc(NC(=O)C3CC3)ncc12 ; CNc1ncc(C2=CCOCC2)c2cc(NC(=O)C3CC3)ncc12]

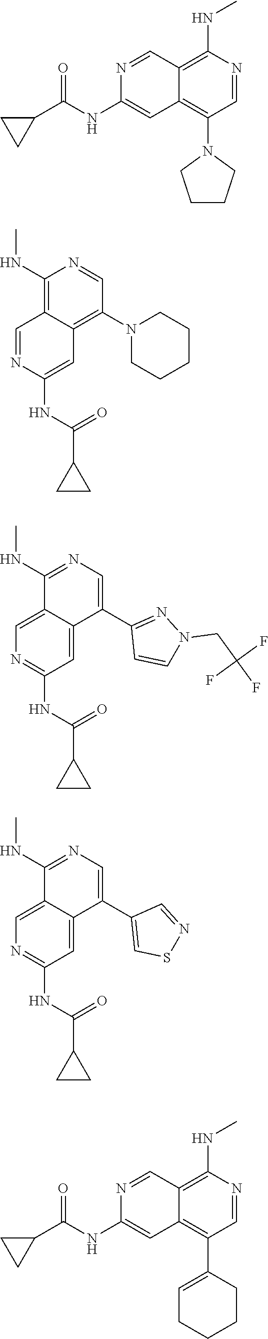 [SMILES: CNc1ncc(N2CCCC2)c2cc(NC(=O)C3CC3)ncc12 ; CNc1ncc(N2CCCCC2)c2cc(NC(=O)C3CC3)ncc12 ; CNc1ncc(-c2ccn(CC(F)(F)F)n2)c2cc(NC(=O)C3CC3)ncc12 ; CNc1ncc(-c2cnsc2)c2cc(NC(=O)C3CC3)ncc12 ; CNc1ncc(C2=CCCCC2)c2cc(NC(=O)C3CC3)ncc12]

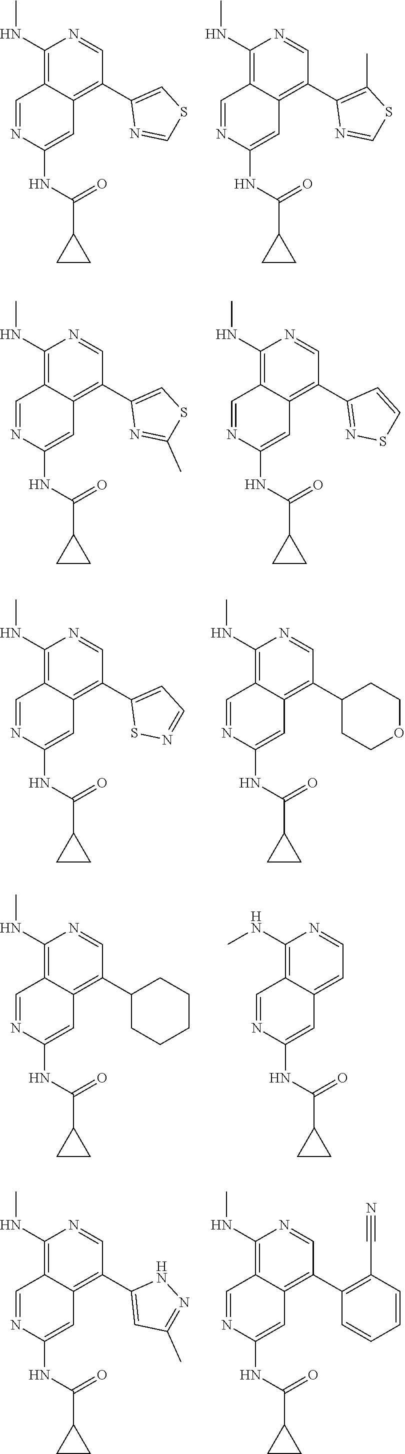

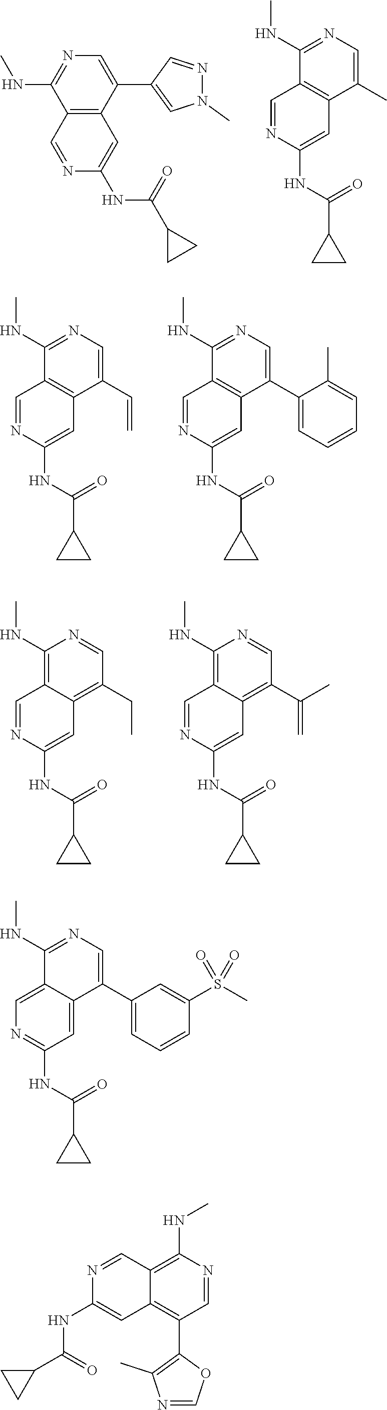

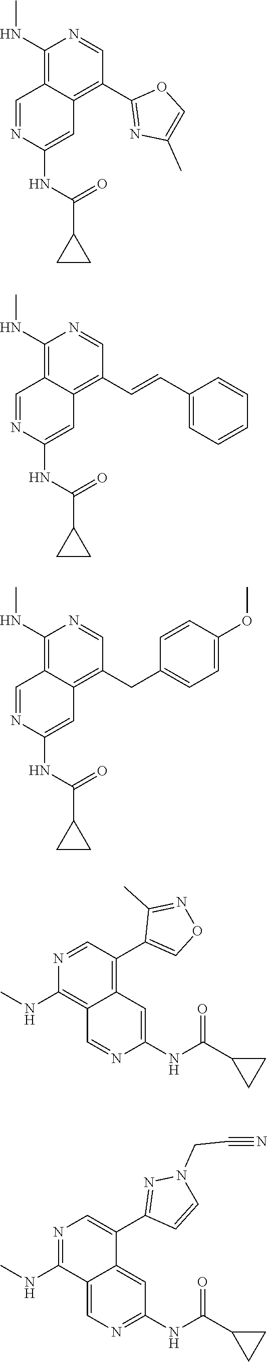

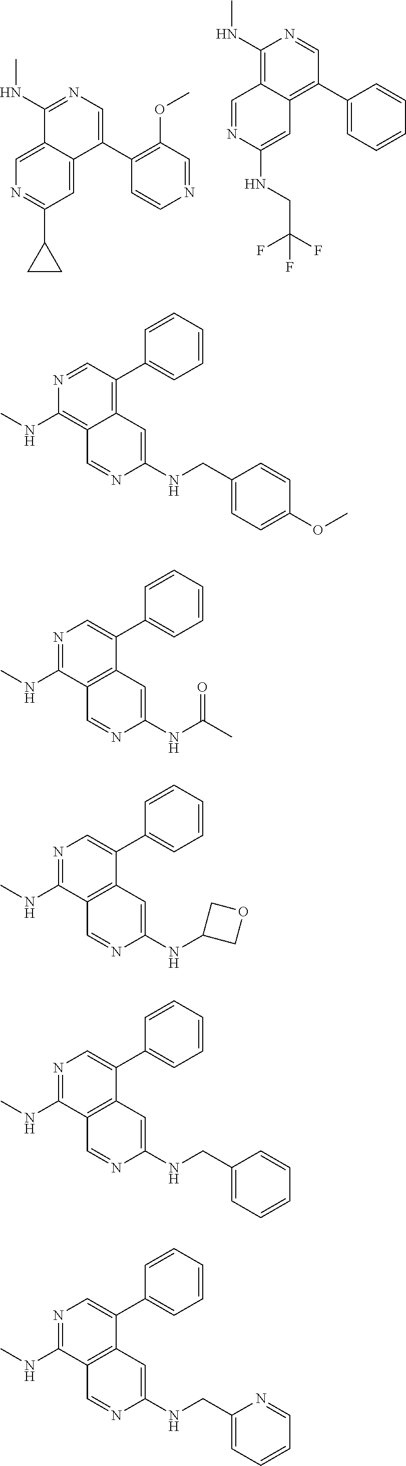

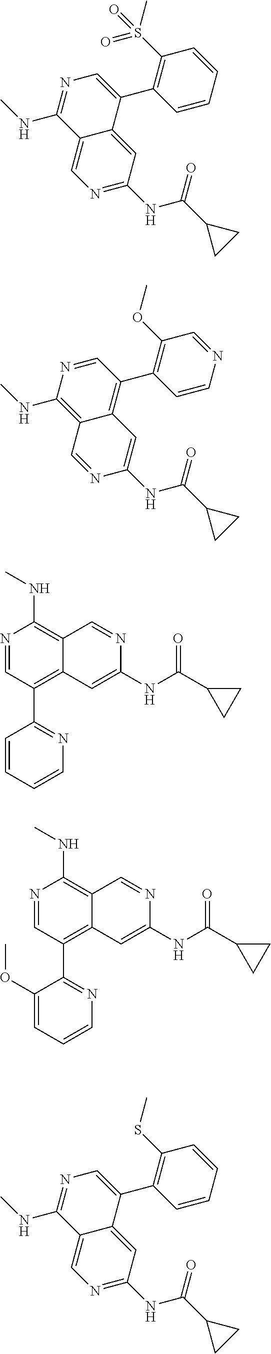

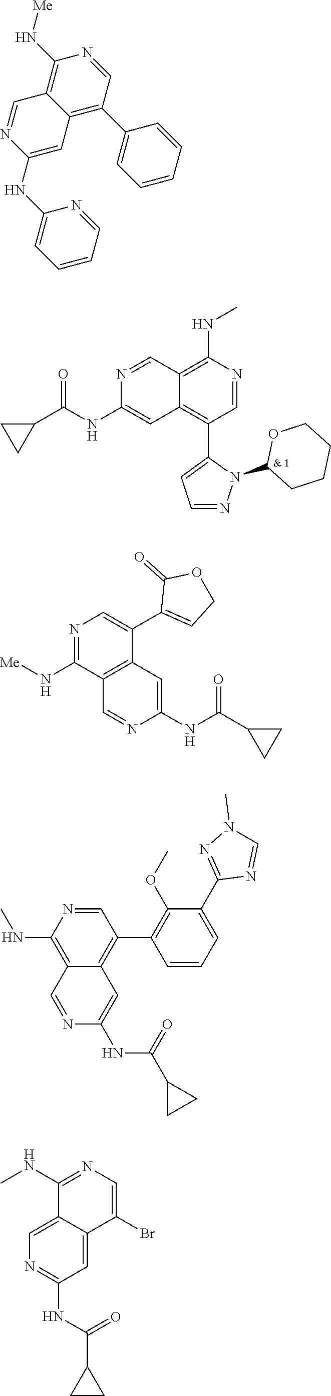

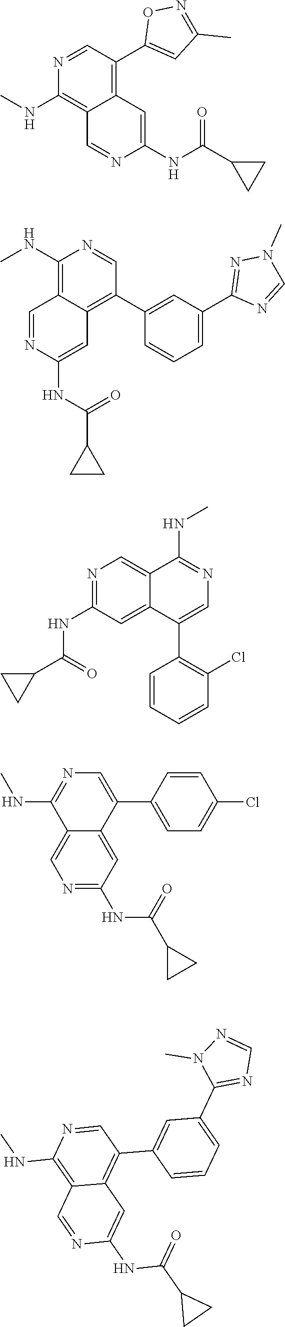 [SMILES: CNc1ncc(-c2cc(C)no2)c2cc(NC(=O)C3CC3)ncc12 ; CNc1ncc(-c2cccc(-c3ncn(C)n3)c2)c2cc(NC(=O)C3CC3)ncc12 ; CNc1ncc(-c2ccccc2Cl)c2cc(NC(=O)C3CC3)ncc12 ; CNc1ncc(-c2ccc(Cl)cc2)c2cc(NC(=O)C3CC3)ncc12 ; CNc1ncc(-c2cccc(-c3ncnn3C)c2)c2cc(NC(=O)C3CC3)ncc12]

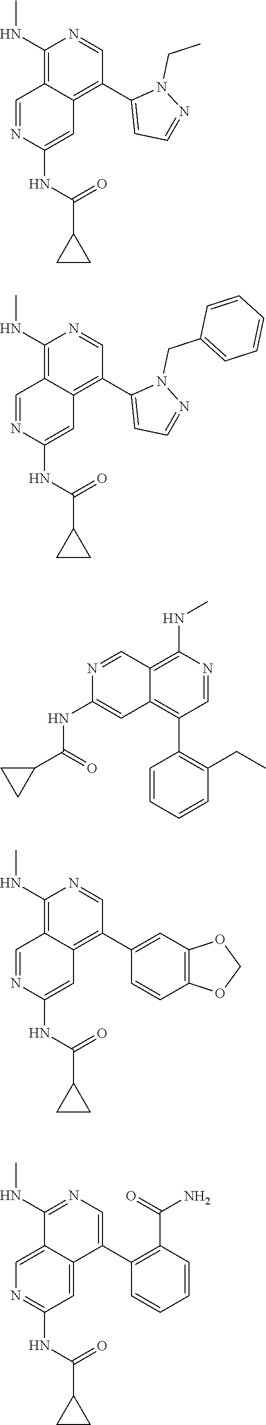 [SMILES: CCn1nccc1-c1cnc(NC)c2cnc(NC(=O)C3CC3)cc12 ; CNc1ncc(-c2ccnn2Cc2ccccc2)c2cc(NC(=O)C3CC3)ncc12 ; CCc1ccccc1-c1cnc(NC)c2cnc(NC(=O)C3CC3)cc12 ; CNc1ncc(-c2ccc3c(c2)OCO3)c2cc(NC(=O)C3CC3)ncc12 ; CNc1ncc(-c2ccccc2C(N)=O)c2cc(NC(=O)C3CC3)ncc12]

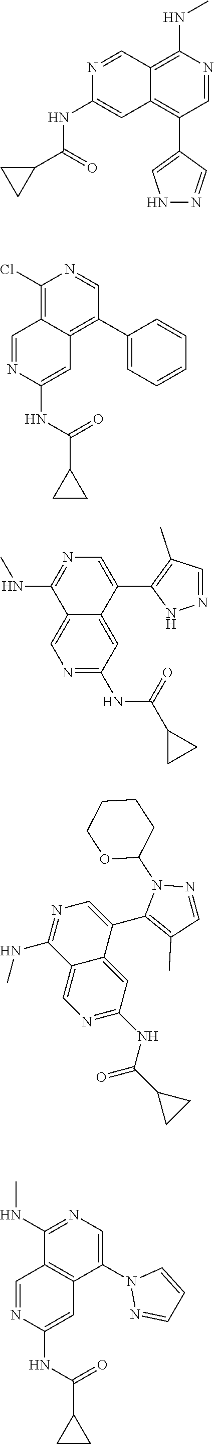 [SMILES: CNc1ncc(-c2cn[nH]c2)c2cc(NC(=O)C3CC3)ncc12 ; O=C(Nc1cc2c(-c3ccccc3)cnc(Cl)c2cn1)C1CC1 ; CNc1ncc(-c2[nH]ncc2C)c2cc(NC(=O)C3CC3)ncc12 ; CNc1ncc(-c2c(C)cnn2C2CCCCO2)c2cc(NC(=O)C3CC3)ncc12 ; CNc1ncc(-n2cccn2)c2cc(NC(=O)C3CC3)ncc12]

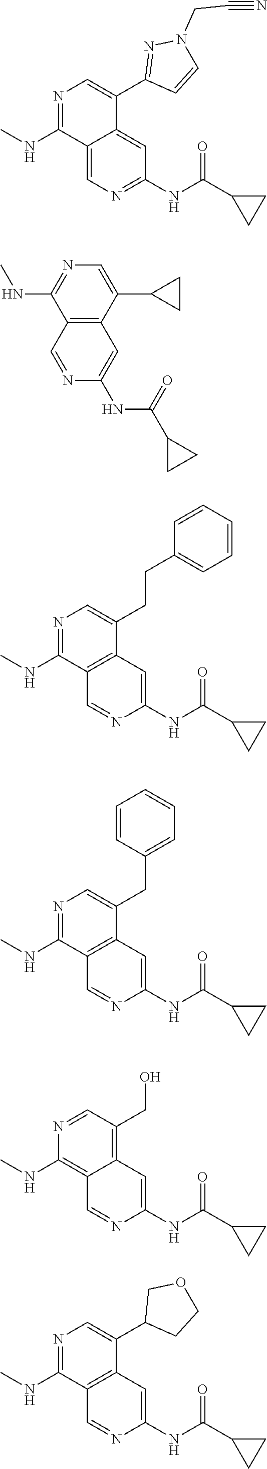 [SMILES: CNc1ncc(-c2ccn(CC#N)n2)c2cc(NC(=O)C3CC3)ncc12 ; CNc1ncc(C2CC2)c2cc(NC(=O)C3CC3)ncc12 ; CNc1ncc(CCc2ccccc2)c2cc(NC(=O)C3CC3)ncc12 ; CNc1ncc(Cc2ccccc2)c2cc(NC(=O)C3CC3)ncc12 ; CNc1ncc(CO)c2cc(NC(=O)C3CC3)ncc12 ; CNc1ncc(C2CCOC2)c2cc(NC(=O)C3CC3)ncc12]

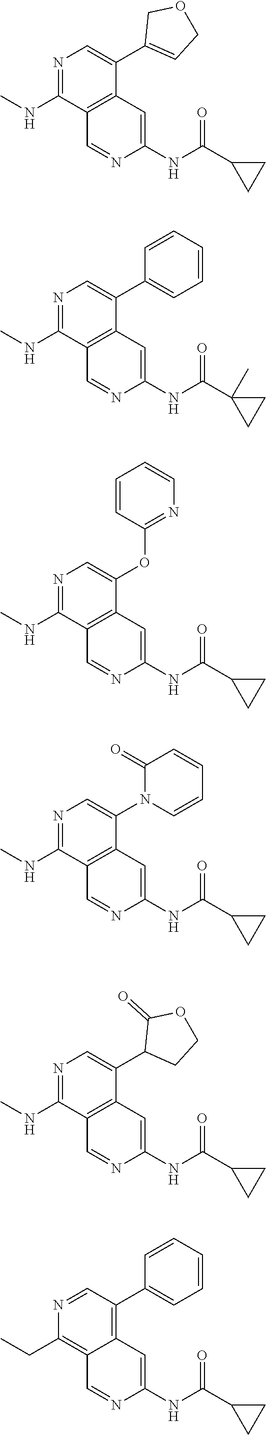 [SMILES: CNc1ncc(C2=CCOC2)c2cc(NC(=O)C3CC3)ncc12 ; CNc1ncc(-c2ccccc2)c2cc(NC(=O)C3(C)CC3)ncc12 ; CNc1ncc(Oc2ccccn2)c2cc(NC(=O)C3CC3)ncc12 ; CNc1ncc(-n2ccccc2=O)c2cc(NC(=O)C3CC3)ncc12 ; CNc1ncc(C2CCOC2=O)c2cc(NC(=O)C3CC3)ncc12 ; CCc1ncc(-c2ccccc2)c2cc(NC(=O)C3CC3)ncc12]

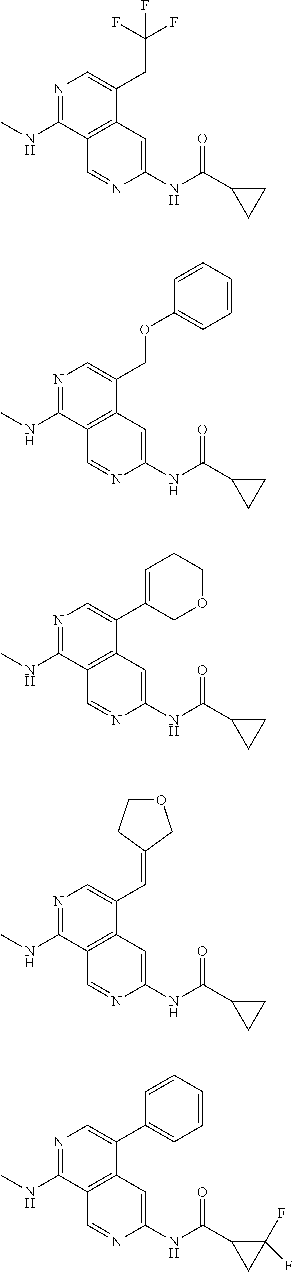 [SMILES: CNc1ncc(CC(F)(F)F)c2cc(NC(=O)C3CC3)ncc12 ; CNc1ncc(COc2ccccc2)c2cc(NC(=O)C3CC3)ncc12 ; CNc1ncc(C2=CCCOC2)c2cc(NC(=O)C3CC3)ncc12 ; CNc1ncc(/C=C2\CCOC2)c2cc(NC(=O)C3CC3)ncc12 ; CNc1ncc(-c2ccccc2)c2cc(NC(=O)C3CC3(F)F)ncc12]

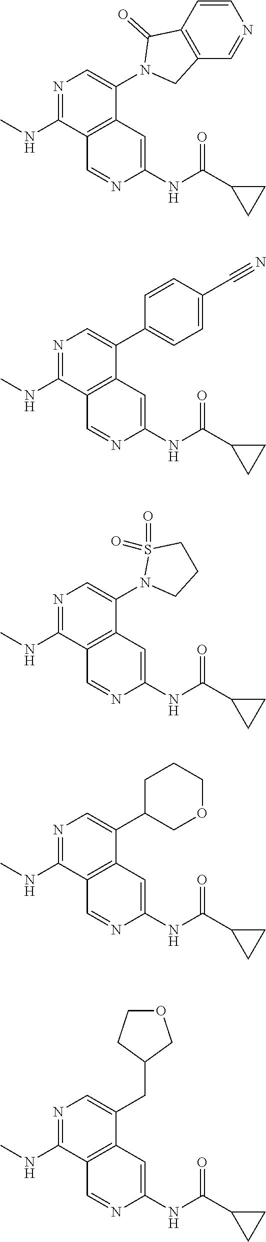 [SMILES: CNc1ncc(N2Cc3cnccc3C2=O)c2cc(NC(=O)C3CC3)ncc12 ; CNc1ncc(-c2ccc(C#N)cc2)c2cc(NC(=O)C3CC3)ncc12 ; CNc1ncc(N2CCCS2(=O)=O)c2cc(NC(=O)C3CC3)ncc12 ; CNc1ncc(C2CCCOC2)c2cc(NC(=O)C3CC3)ncc12 ; CNc1ncc(CC2CCOC2)c2cc(NC(=O)C3CC3)ncc12]

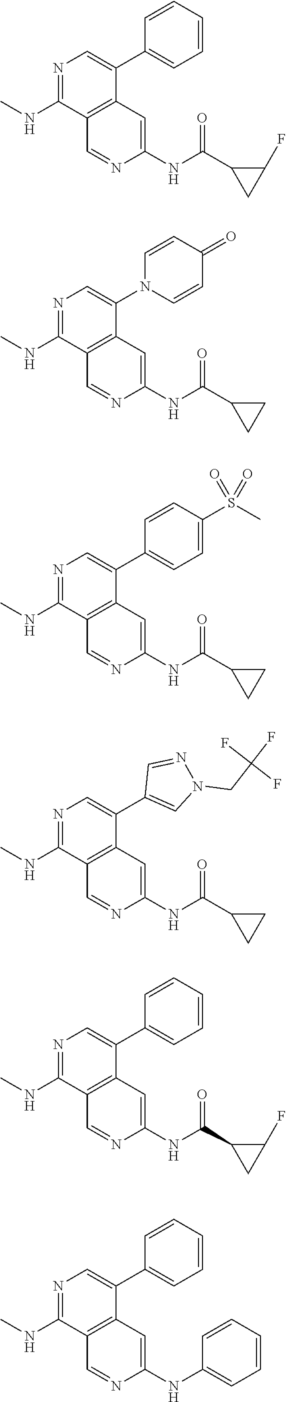

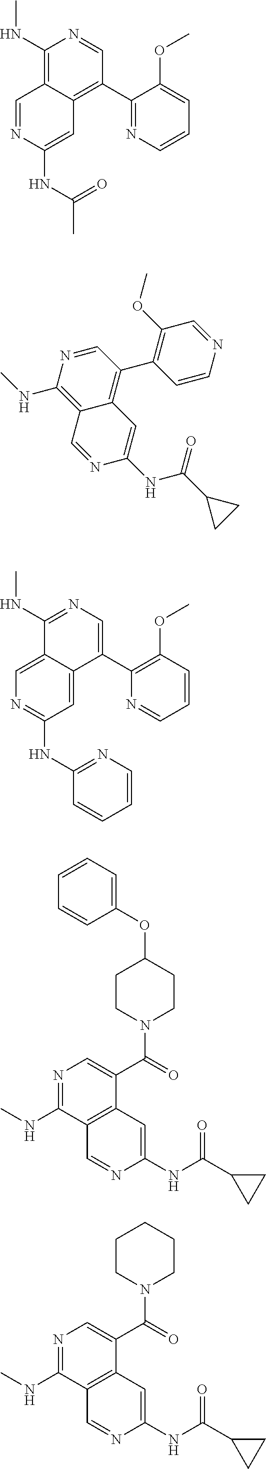

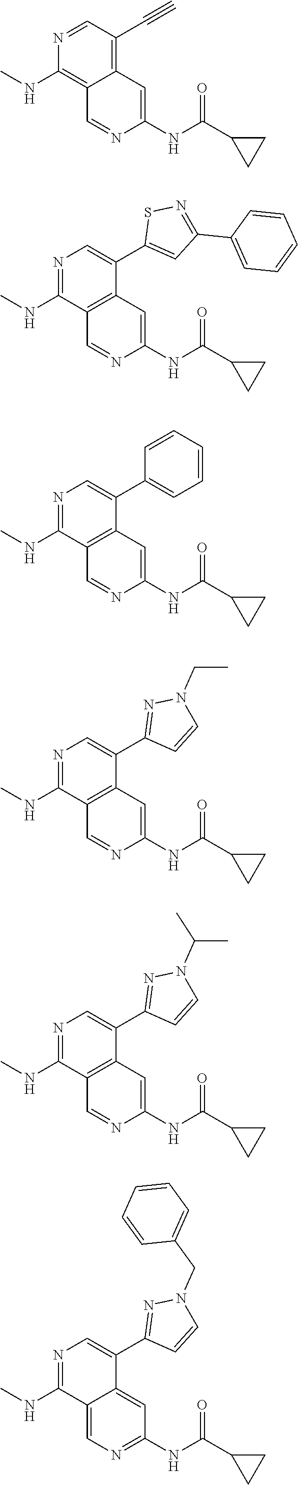

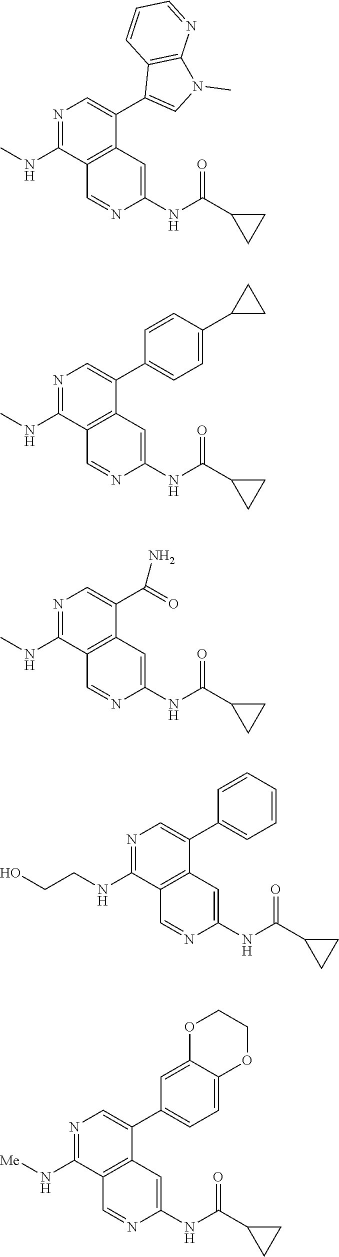

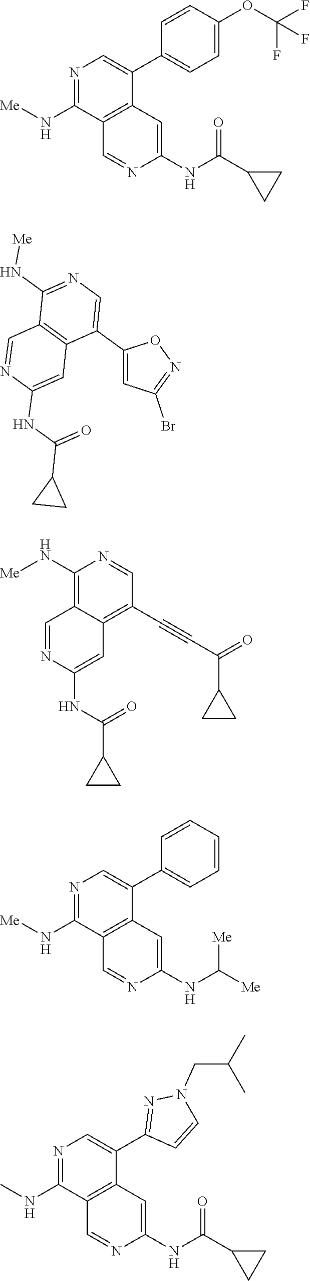 [SMILES: CNc1ncc(-c2ccc(OC(F)(F)F)cc2)c2cc(NC(=O)C3CC3)ncc12 ; CNc1ncc(-c2cc(Br)no2)c2cc(NC(=O)C3CC3)ncc12 ; CNc1ncc(C#CC(=O)C2CC2)c2cc(NC(=O)C3CC3)ncc12 ; CNc1ncc(-c2ccccc2)c2cc(NC(C)C)ncc12 ; CNc1ncc(-c2ccn(CC(C)C)n2)c2cc(NC(=O)C3CC3)ncc12]

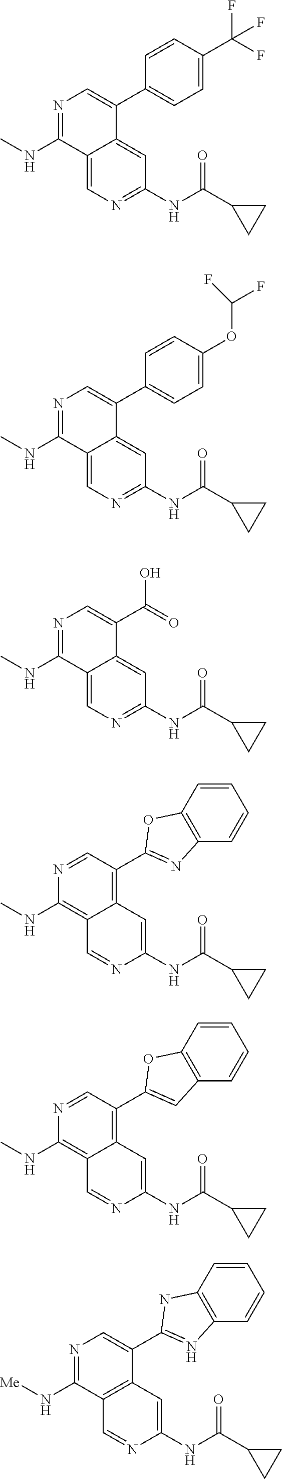

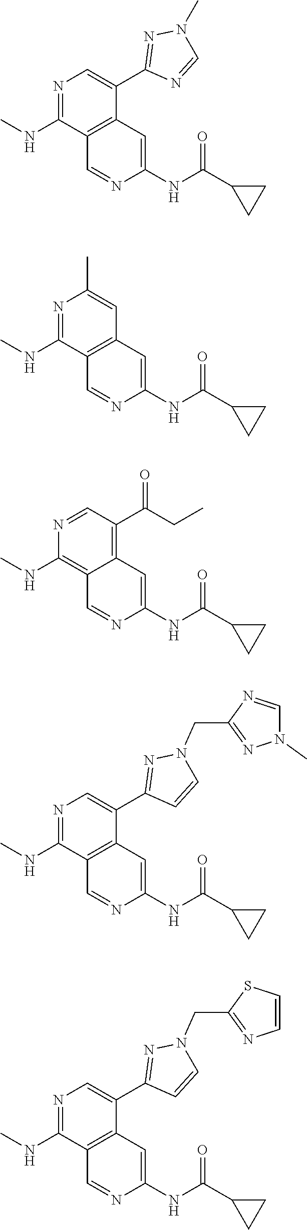

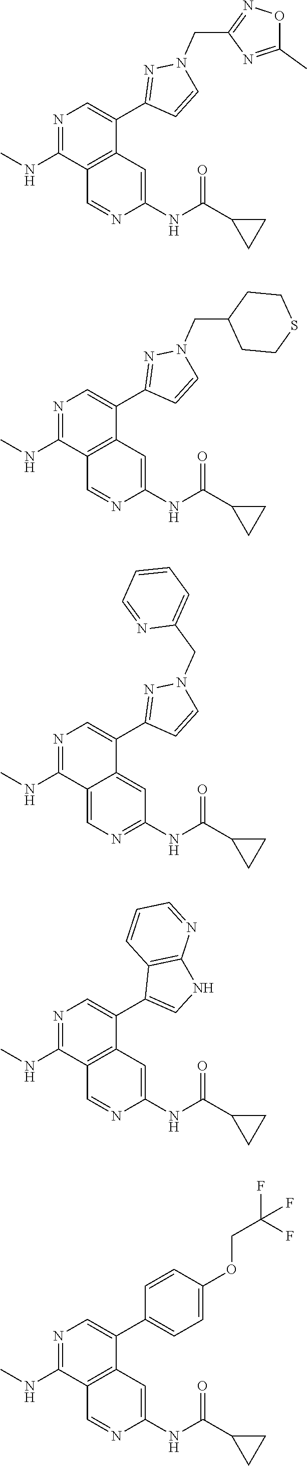

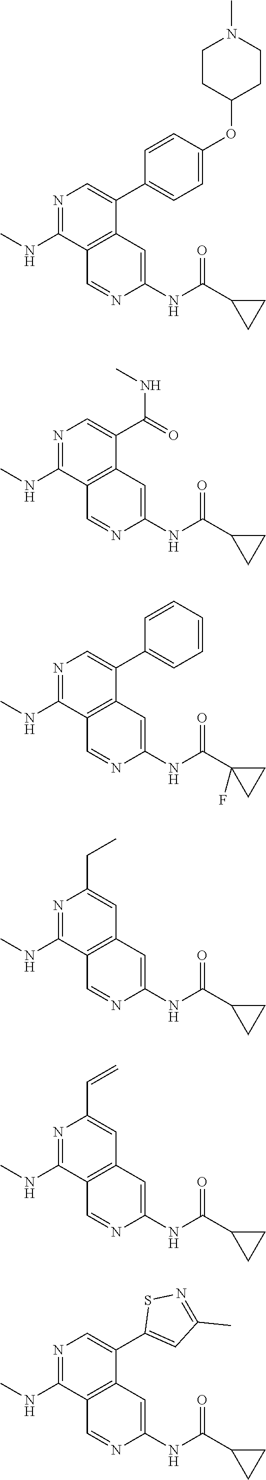 [SMILES: CNc1ncc(-c2ccc(OC3CCN(C)CC3)cc2)c2cc(NC(=O)C3CC3)ncc12 ; CNC(=O)c1cnc(NC)c2cnc(NC(=O)C3CC3)cc12 ; CNc1ncc(-c2ccccc2)c2cc(NC(=O)C3(F)CC3)ncc12 ; CCc1cc2cc(NC(=O)C3CC3)ncc2c(NC)n1 ; C=Cc1cc2cc(NC(=O)C3CC3)ncc2c(NC)n1 ; CNc1ncc(-c2cc(C)ns2)c2cc(NC(=O)C3CC3)ncc12]

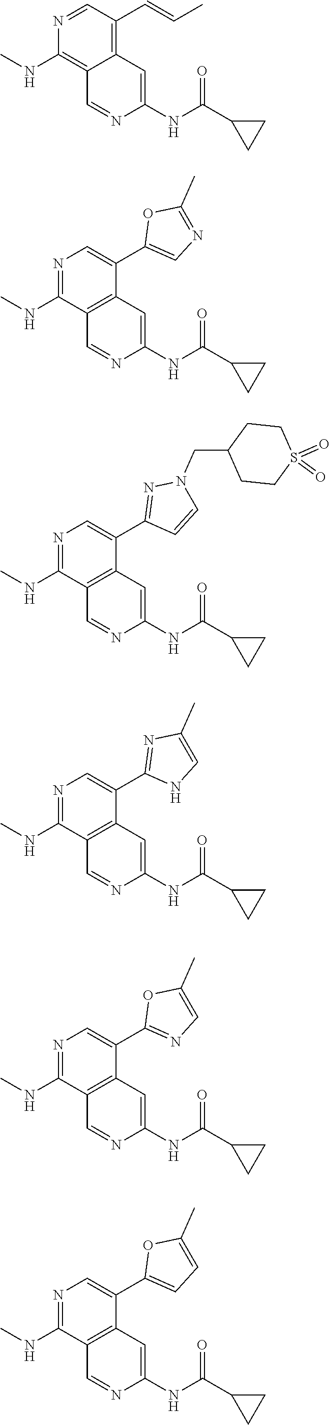

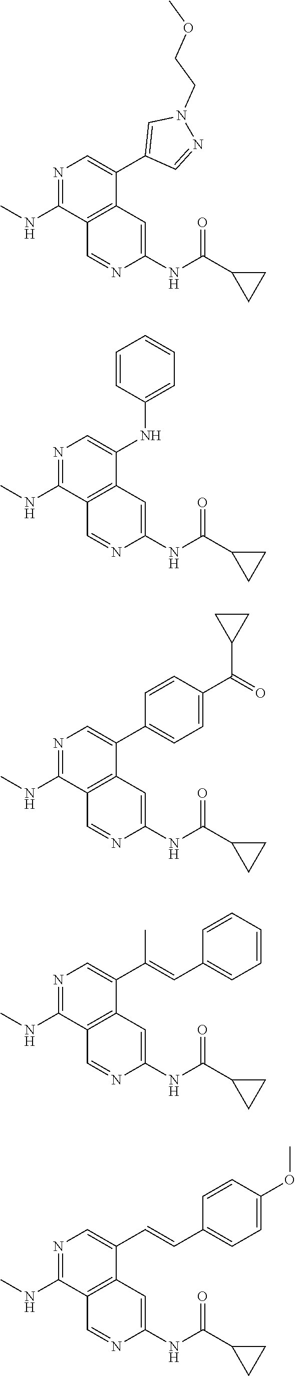

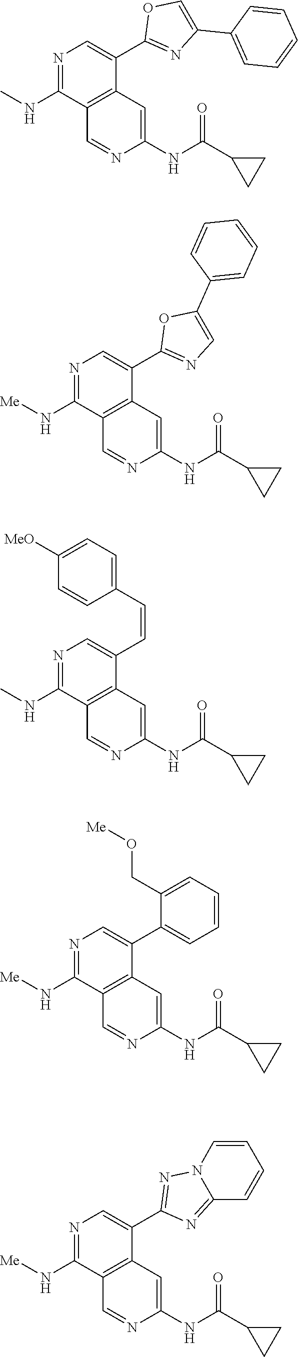

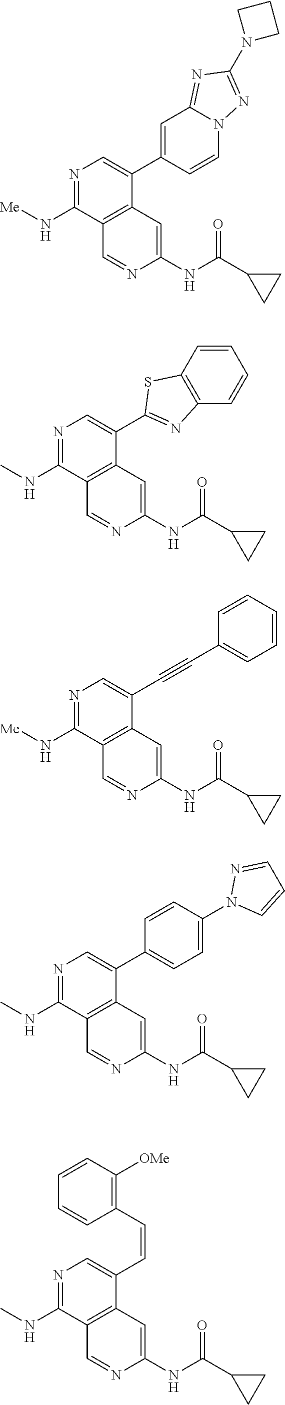

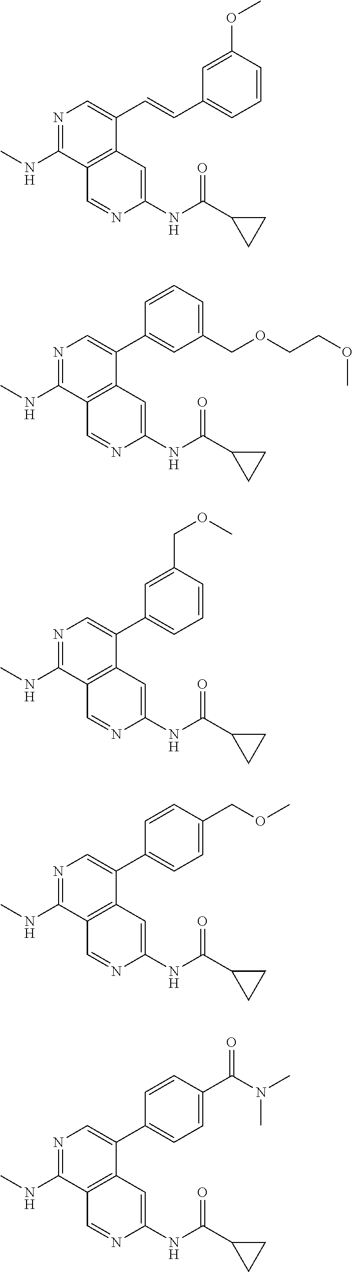

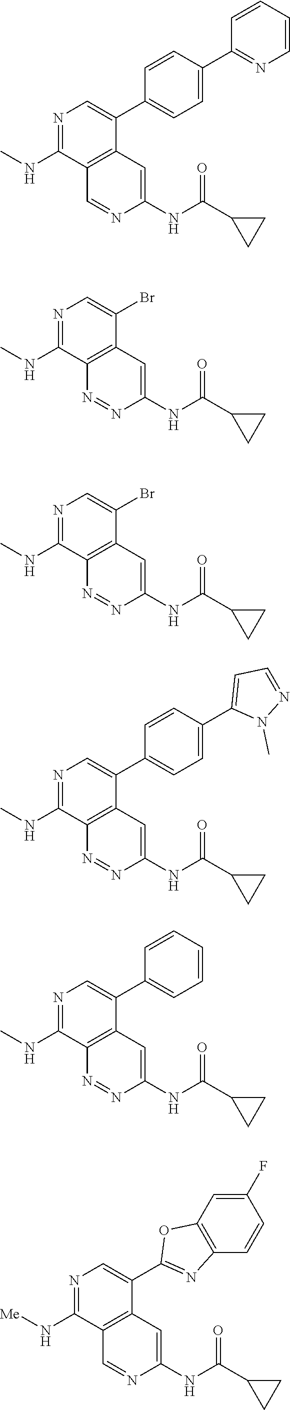

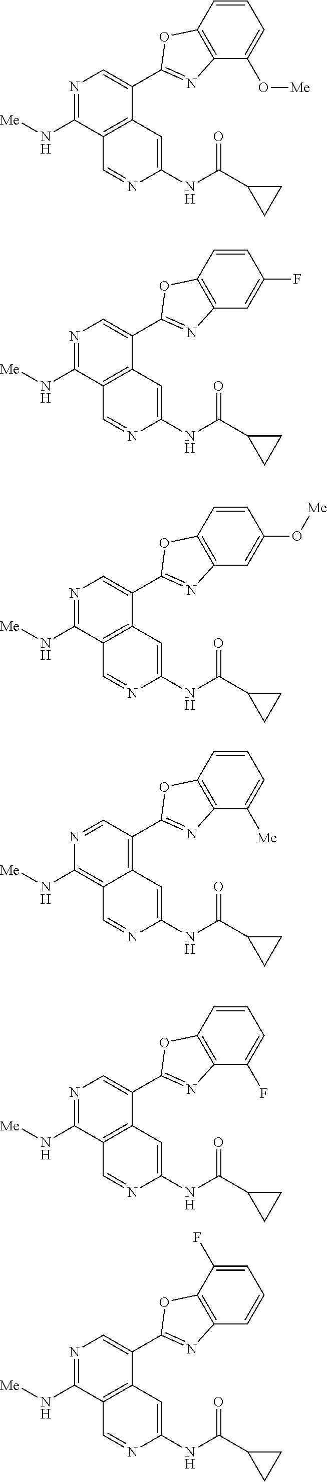

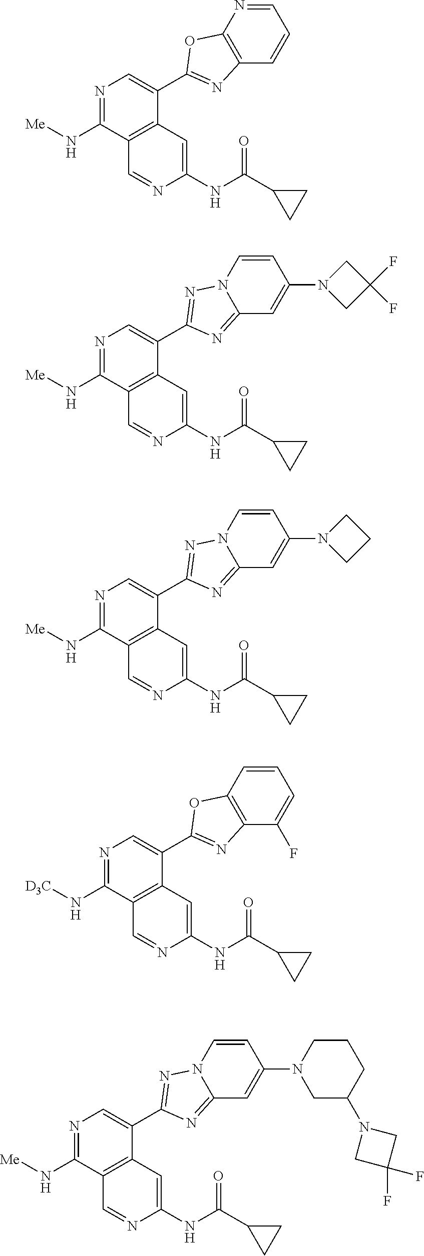

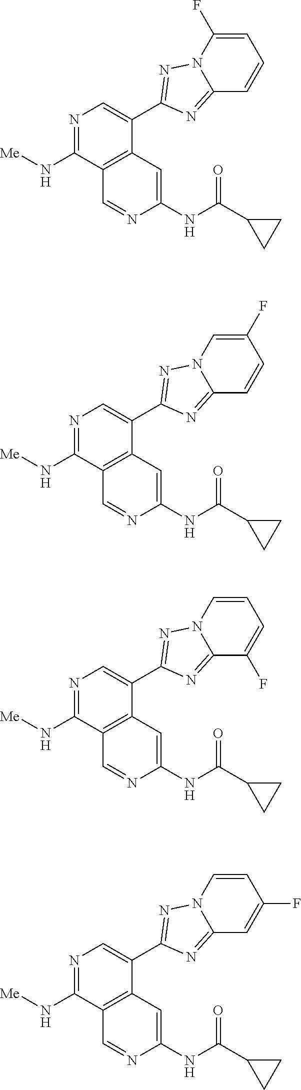 [SMILES: CNc1ncc(-c2nc3cccc(F)n3n2)c2cc(NC(=O)C3CC3)ncc12 ; CNc1ncc(-c2nc3ccc(F)cn3n2)c2cc(NC(=O)C3CC3)ncc12 ; CNc1ncc(-c2nc3c(F)cccn3n2)c2cc(NC(=O)C3CC3)ncc12 ; CNc1ncc(-c2nc3cc(F)ccn3n2)c2cc(NC(=O)C3CC3)ncc12]

18. The compound of claim 1, wherein the compound is selected from the group consisting of:

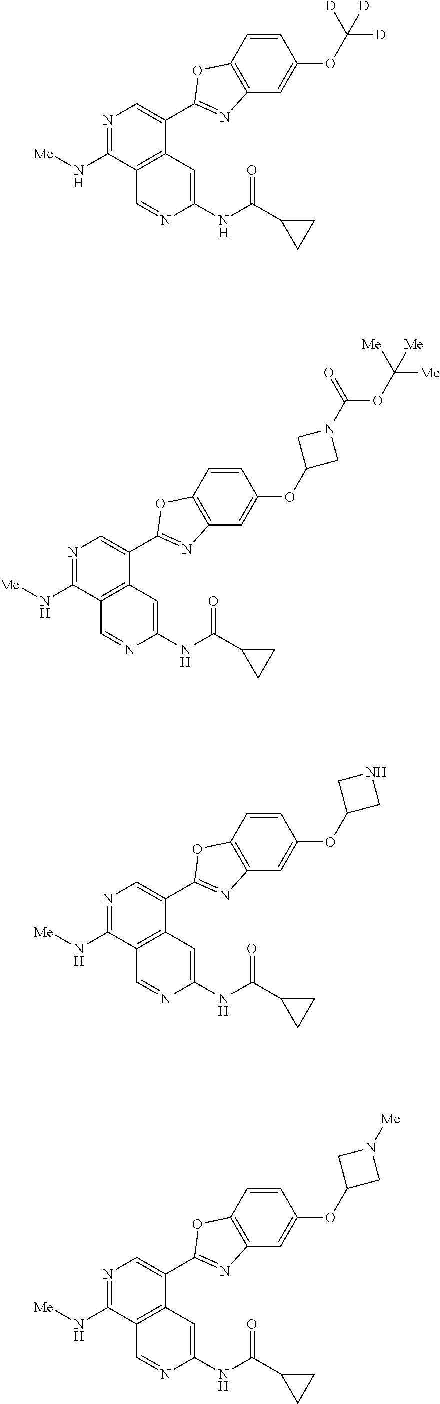 [SMILES: [2H]C([2H])([2H])Oc1ccc2oc(-c3cnc(NC)c4cnc(NC(=O)C5CC5)cc34)nc2c1 ; CNc1ncc(-c2nc3cc(OC4CN(C(=O)OC(C)(C)C)C4)ccc3o2)c2cc(NC(=O)C3CC3)ncc12 ; CNc1ncc(-c2nc3cc(OC4CNC4)ccc3o2)c2cc(NC(=O)C3CC3)ncc12 ; CNc1ncc(-c2nc3cc(OC4CN(C)C4)ccc3o2)c2cc(NC(=O)C3CC3)ncc12]

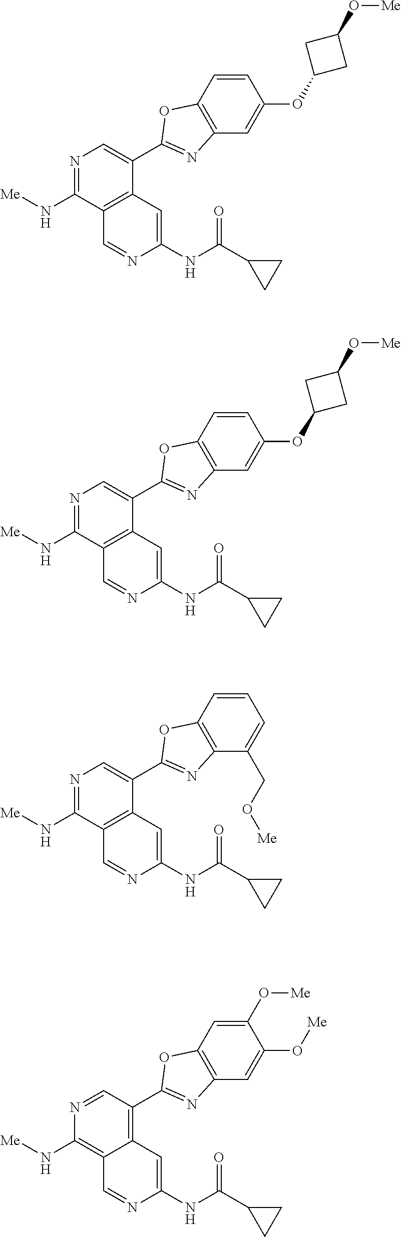 [SMILES: CNc1ncc(-c2nc3cc(O[C@H]4C[C@H](OC)C4)ccc3o2)c2cc(NC(=O)C3CC3)ncc12 ; CNc1ncc(-c2nc3cc(O[C@H]4C[C@@H](OC)C4)ccc3o2)c2cc(NC(=O)C3CC3)ncc12 ; CNc1ncc(-c2nc3c(COC)cccc3o2)c2cc(NC(=O)C3CC3)ncc12 ; CNc1ncc(-c2nc3cc(OC)c(OC)cc3o2)c2cc(NC(=O)C3CC3)ncc12]

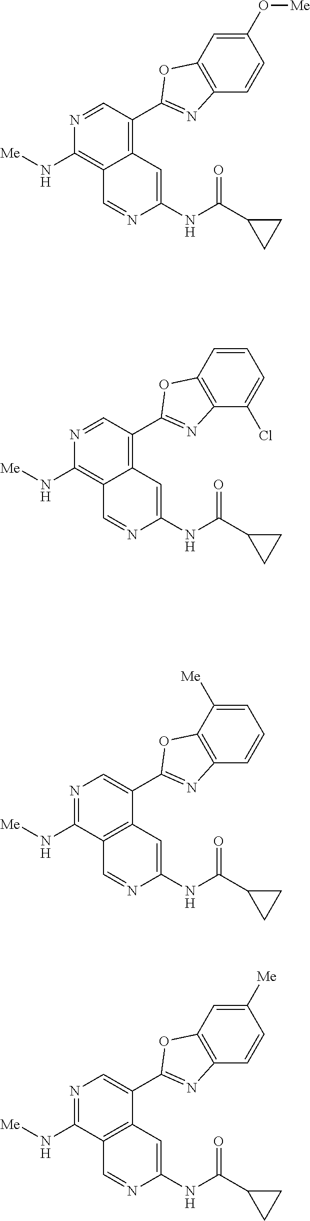 [SMILES: CNc1ncc(-c2nc3ccc(OC)cc3o2)c2cc(NC(=O)C3CC3)ncc12 ; CNc1ncc(-c2nc3c(Cl)cccc3o2)c2cc(NC(=O)C3CC3)ncc12 ; CNc1ncc(-c2nc3cccc(C)c3o2)c2cc(NC(=O)C3CC3)ncc12 ; CNc1ncc(-c2nc3ccc(C)cc3o2)c2cc(NC(=O)C3CC3)ncc12]

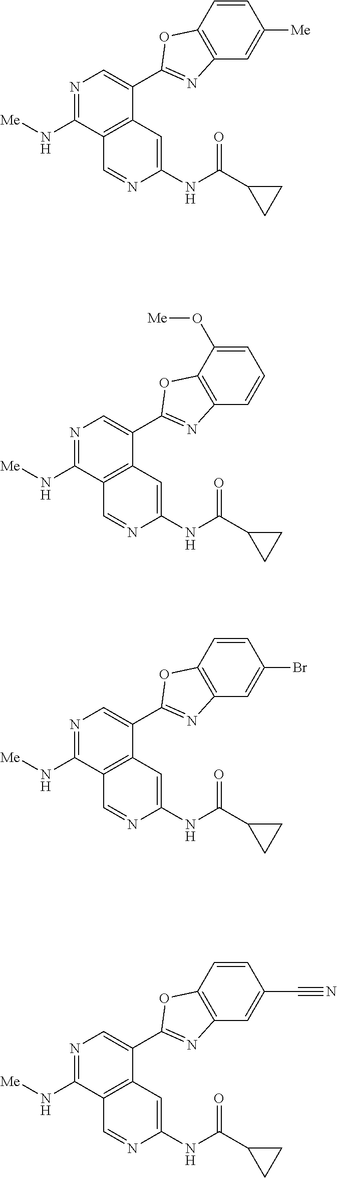 [SMILES: CNc1ncc(-c2nc3cc(C)ccc3o2)c2cc(NC(=O)C3CC3)ncc12 ; CNc1ncc(-c2nc3cccc(OC)c3o2)c2cc(NC(=O)C3CC3)ncc12 ; CNc1ncc(-c2nc3cc(Br)ccc3o2)c2cc(NC(=O)C3CC3)ncc12 ; CNc1ncc(-c2nc3cc(C#N)ccc3o2)c2cc(NC(=O)C3CC3)ncc12]

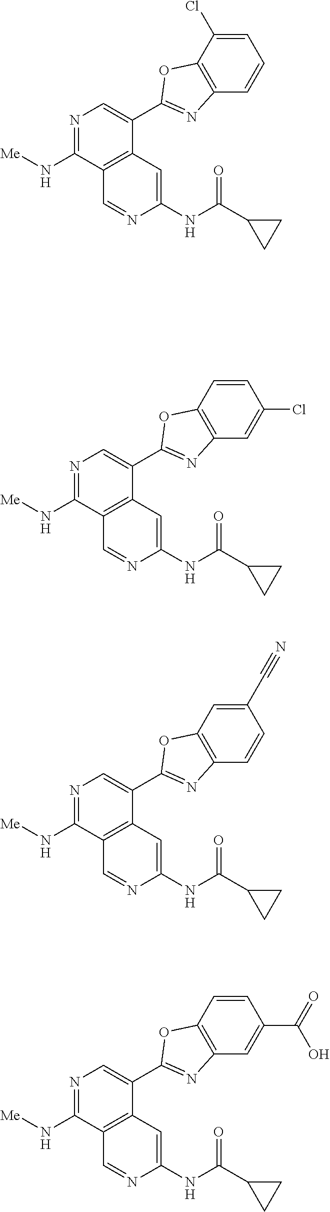 [SMILES: CNc1ncc(-c2nc3cccc(Cl)c3o2)c2cc(NC(=O)C3CC3)ncc12 ; CNc1ncc(-c2nc3cc(Cl)ccc3o2)c2cc(NC(=O)C3CC3)ncc12 ; CNc1ncc(-c2nc3ccc(C#N)cc3o2)c2cc(NC(=O)C3CC3)ncc12 ; CNc1ncc(-c2nc3cc(C(=O)O)ccc3o2)c2cc(NC(=O)C3CC3)ncc12]

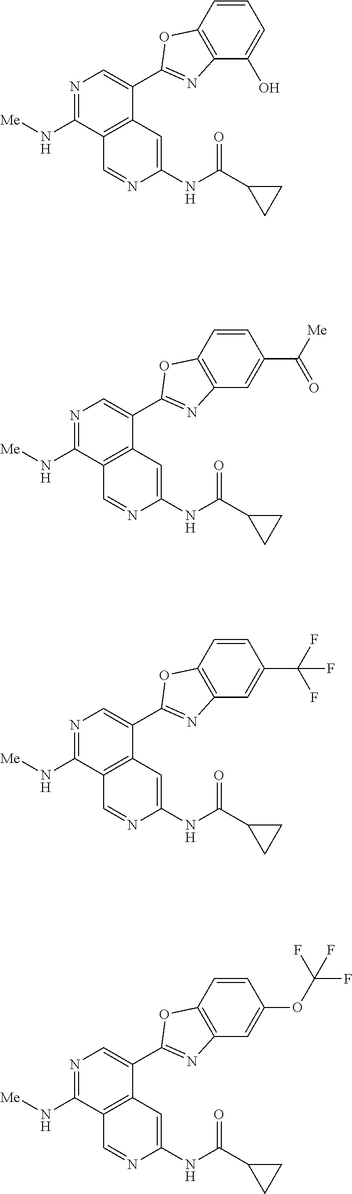 [SMILES: CNc1ncc(-c2nc3c(O)cccc3o2)c2cc(NC(=O)C3CC3)ncc12 ; CNc1ncc(-c2nc3cc(C(C)=O)ccc3o2)c2cc(NC(=O)C3CC3)ncc12 ; CNc1ncc(-c2nc3cc(C(F)(F)F)ccc3o2)c2cc(NC(=O)C3CC3)ncc12 ; CNc1ncc(-c2nc3cc(OC(F)(F)F)ccc3o2)c2cc(NC(=O)C3CC3)ncc12]

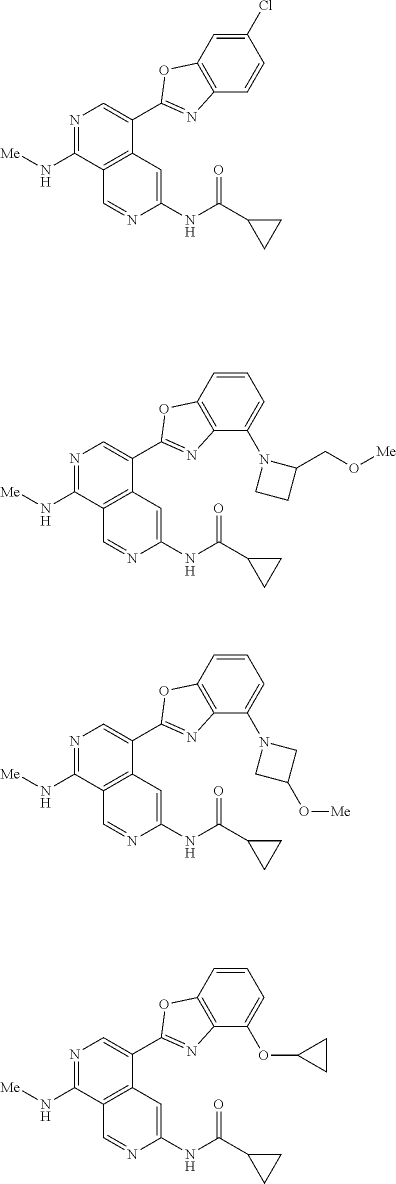 [SMILES: CNc1ncc(-c2nc3ccc(Cl)cc3o2)c2cc(NC(=O)C3CC3)ncc12 ; CNc1ncc(-c2nc3c(N4CCC4COC)cccc3o2)c2cc(NC(=O)C3CC3)ncc12 ; CNc1ncc(-c2nc3c(N4CC(OC)C4)cccc3o2)c2cc(NC(=O)C3CC3)ncc12 ; CNc1ncc(-c2nc3c(OC4CC4)cccc3o2)c2cc(NC(=O)C3CC3)ncc12]

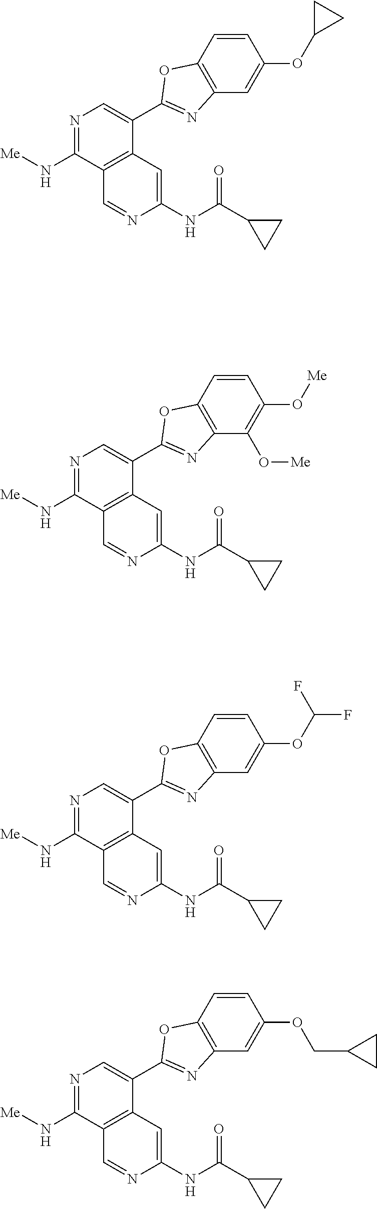 [SMILES: CNc1ncc(-c2nc3cc(OC4CC4)ccc3o2)c2cc(NC(=O)C3CC3)ncc12 ; CNc1ncc(-c2nc3c(OC)c(OC)ccc3o2)c2cc(NC(=O)C3CC3)ncc12 ; CNc1ncc(-c2nc3cc(OC(F)F)ccc3o2)c2cc(NC(=O)C3CC3)ncc12 ; CNc1ncc(-c2nc3cc(OCC4CC4)ccc3o2)c2cc(NC(=O)C3CC3)ncc12]

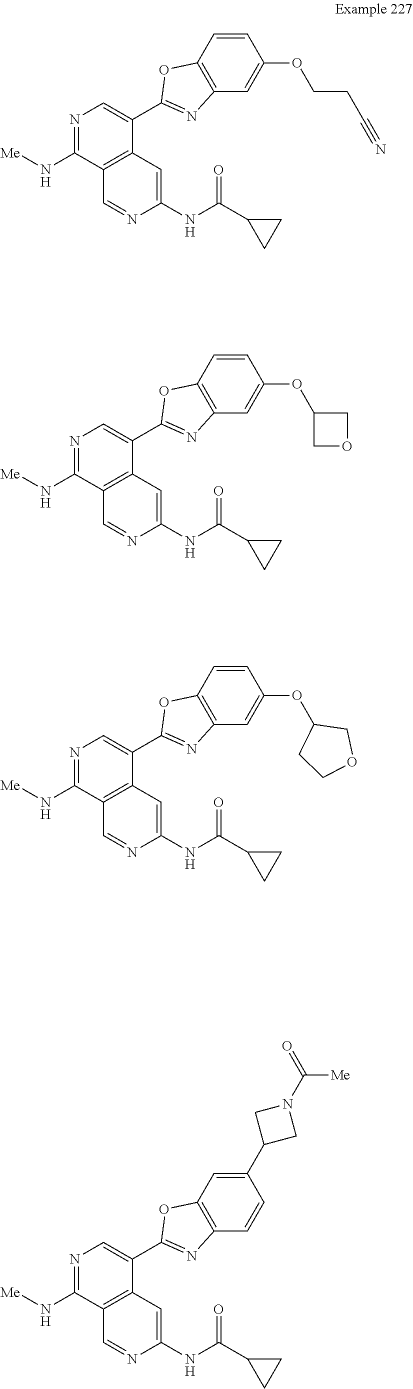 [SMILES: CNc1ncc(-c2nc3cc(OCCC#N)ccc3o2)c2cc(NC(=O)C3CC3)ncc12 ; CNc1ncc(-c2nc3cc(OC4COC4)ccc3o2)c2cc(NC(=O)C3CC3)ncc12 ; CNc1ncc(-c2nc3cc(OC4CCOC4)ccc3o2)c2cc(NC(=O)C3CC3)ncc12 ; CNc1ncc(-c2nc3ccc(C4CN(C(C)=O)C4)cc3o2)c2cc(NC(=O)C3CC3)ncc12]

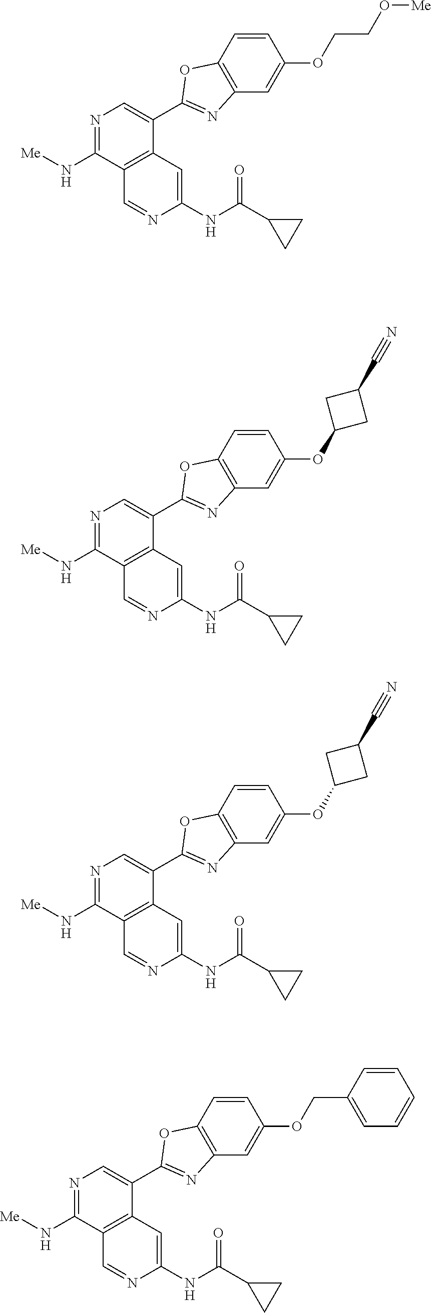 [SMILES: CNc1ncc(-c2nc3cc(OCCOC)ccc3o2)c2cc(NC(=O)C3CC3)ncc12 ; CNc1ncc(-c2nc3cc(O[C@H]4C[C@@H](C#N)C4)ccc3o2)c2cc(NC(=O)C3CC3)ncc12 ; CNc1ncc(-c2nc3cc(O[C@H]4C[C@H](C#N)C4)ccc3o2)c2cc(NC(=O)C3CC3)ncc12 ; CNc1ncc(-c2nc3cc(OCc4ccccc4)ccc3o2)c2cc(NC(=O)C3CC3)ncc12]

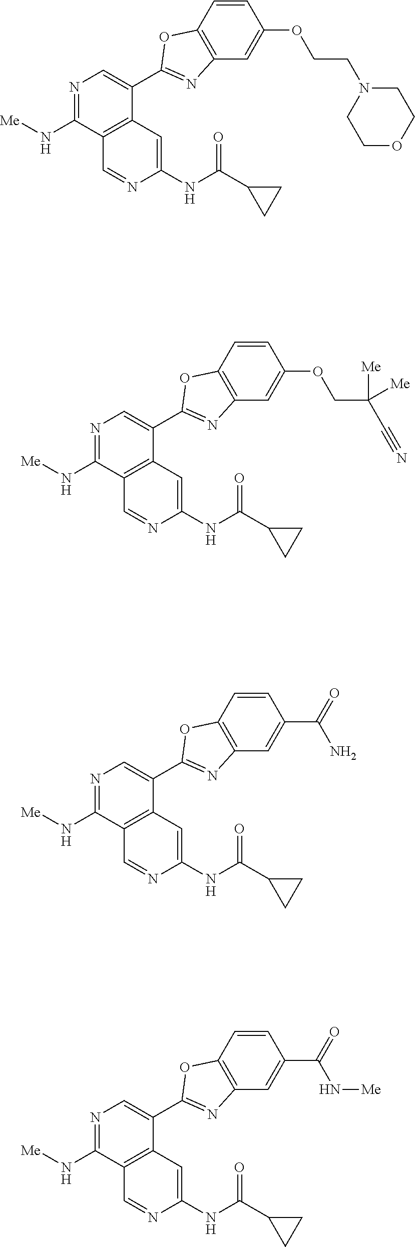 [SMILES: CNc1ncc(-c2nc3cc(OCCN4CCOCC4)ccc3o2)c2cc(NC(=O)C3CC3)ncc12 ; CNc1ncc(-c2nc3cc(OCC(C)(C)C#N)ccc3o2)c2cc(NC(=O)C3CC3)ncc12 ; CNc1ncc(-c2nc3cc(C(N)=O)ccc3o2)c2cc(NC(=O)C3CC3)ncc12 ; CNC(=O)c1ccc2oc(-c3cnc(NC)c4cnc(NC(=O)C5CC5)cc34)nc2c1]

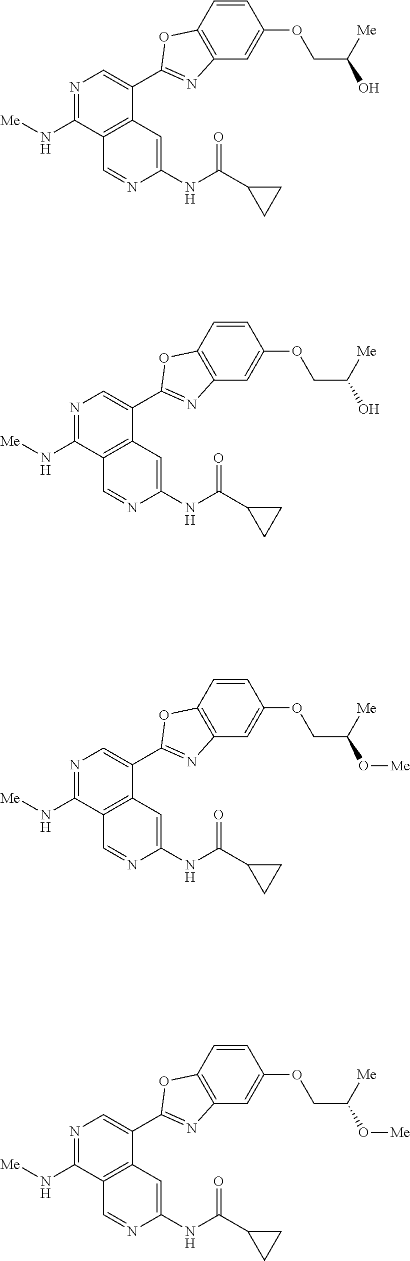 [SMILES: CNc1ncc(-c2nc3cc(OC[C@@H](C)O)ccc3o2)c2cc(NC(=O)C3CC3)ncc12 ; CNc1ncc(-c2nc3cc(OC[C@H](C)O)ccc3o2)c2cc(NC(=O)C3CC3)ncc12 ; CNc1ncc(-c2nc3cc(OC[C@@H](C)OC)ccc3o2)c2cc(NC(=O)C3CC3)ncc12 ; CNc1ncc(-c2nc3cc(OC[C@H](C)OC)ccc3o2)c2cc(NC(=O)C3CC3)ncc12]

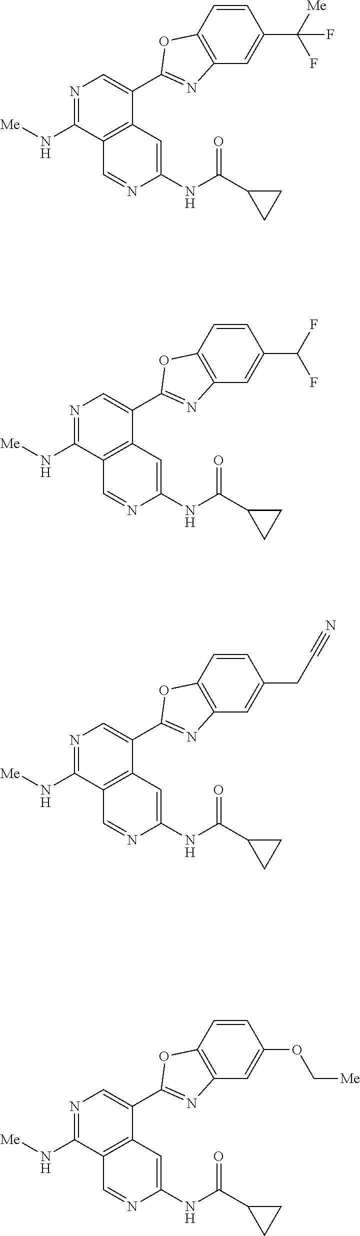 [SMILES: CNc1ncc(-c2nc3cc(C(C)(F)F)ccc3o2)c2cc(NC(=O)C3CC3)ncc12 ; CNc1ncc(-c2nc3cc(C(F)F)ccc3o2)c2cc(NC(=O)C3CC3)ncc12 ; CNc1ncc(-c2nc3cc(CC#N)ccc3o2)c2cc(NC(=O)C3CC3)ncc12 ; CCOc1ccc2oc(-c3cnc(NC)c4cnc(NC(=O)C5CC5)cc34)nc2c1]

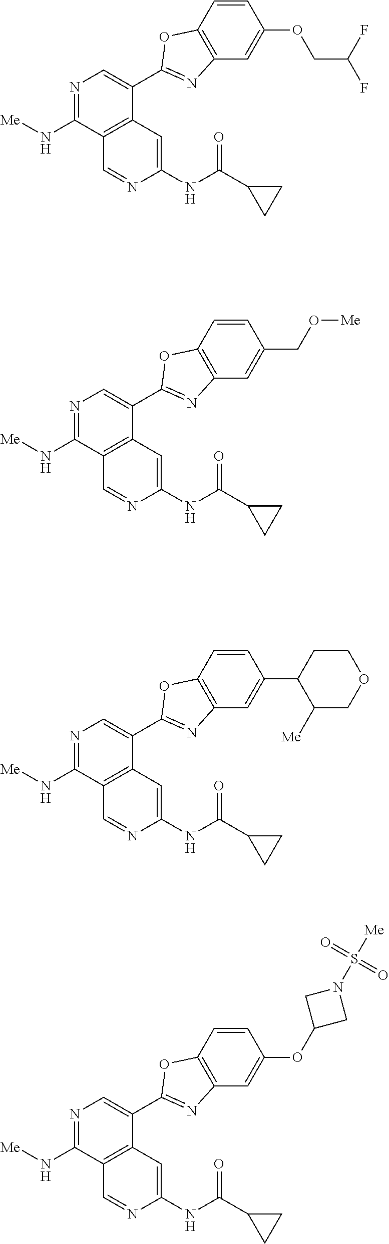 [SMILES: CNc1ncc(-c2nc3cc(OCC(F)F)ccc3o2)c2cc(NC(=O)C3CC3)ncc12 ; CNc1ncc(-c2nc3cc(COC)ccc3o2)c2cc(NC(=O)C3CC3)ncc12 ; CNc1ncc(-c2nc3cc(C4CCOCC4C)ccc3o2)c2cc(NC(=O)C3CC3)ncc12 ; CNc1ncc(-c2nc3cc(OC4CN(S(C)(=O)=O)C4)ccc3o2)c2cc(NC(=O)C3CC3)ncc12]

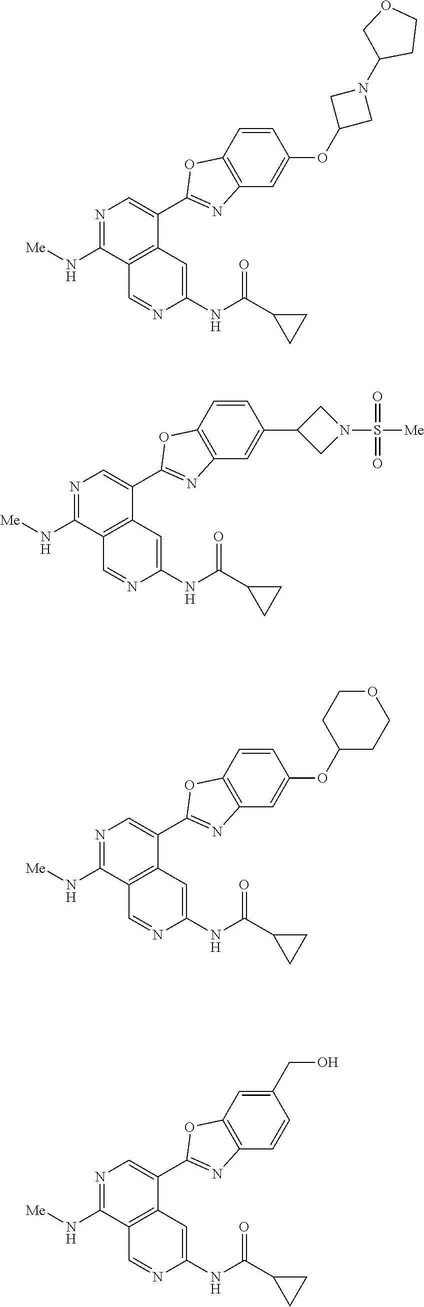 [SMILES: CNc1ncc(-c2nc3cc(OC4CN(C5CCOC5)C4)ccc3o2)c2cc(NC(=O)C3CC3)ncc12 ; CNc1ncc(-c2nc3cc(C4CN(S(C)(=O)=O)C4)ccc3o2)c2cc(NC(=O)C3CC3)ncc12 ; CNc1ncc(-c2nc3cc(OC4CCOCC4)ccc3o2)c2cc(NC(=O)C3CC3)ncc12 ; CNc1ncc(-c2nc3ccc(CO)cc3o2)c2cc(NC(=O)C3CC3)ncc12]

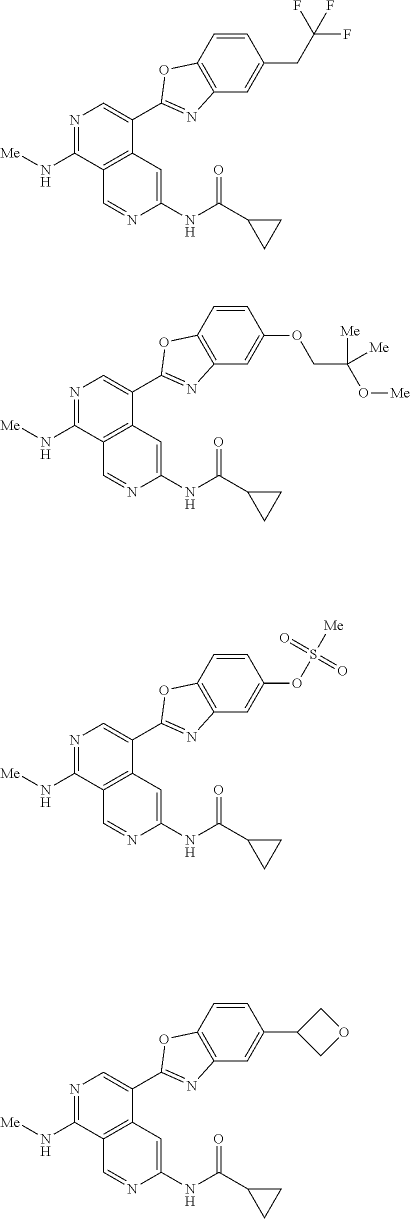 [SMILES: CNc1ncc(-c2nc3cc(CC(F)(F)F)ccc3o2)c2cc(NC(=O)C3CC3)ncc12 ; CNc1ncc(-c2nc3cc(OCC(C)(C)OC)ccc3o2)c2cc(NC(=O)C3CC3)ncc12 ; CNc1ncc(-c2nc3cc(OS(C)(=O)=O)ccc3o2)c2cc(NC(=O)C3CC3)ncc12 ; CNc1ncc(-c2nc3cc(C4COC4)ccc3o2)c2cc(NC(=O)C3CC3)ncc12]

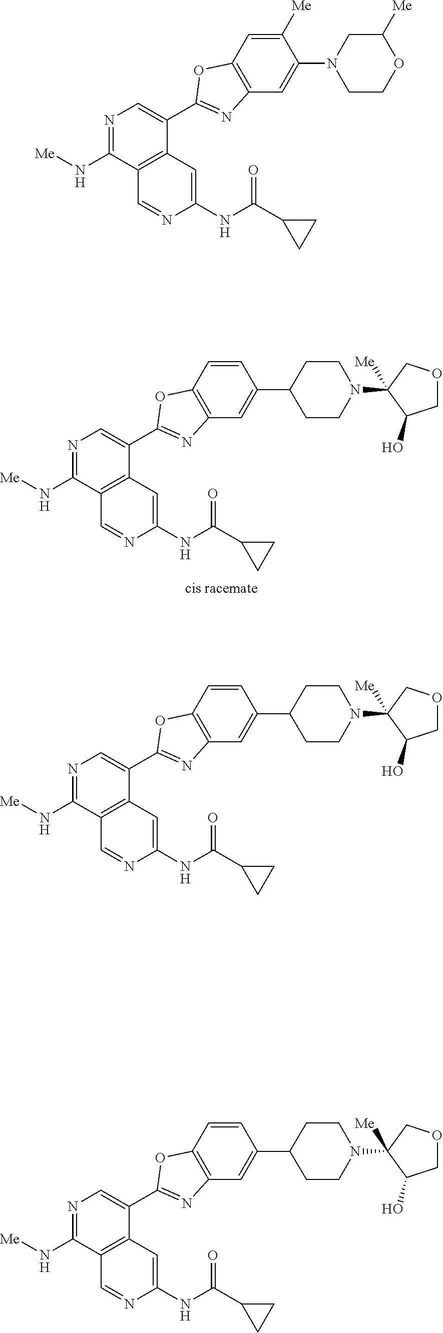 [SMILES: CNc1ncc(-c2nc3cc(N4CCOC(C)C4)c(C)cc3o2)c2cc(NC(=O)C3CC3)ncc12 ; CNc1ncc(-c2nc3cc(C4CCN([C@@]5(C)COC[C@H]5O)CC4)ccc3o2)c2cc(NC(=O)C3CC3)ncc12 ; CNc1ncc(-c2nc3cc(C4CCN([C@@]5(C)COC[C@H]5O)CC4)ccc3o2)c2cc(NC(=O)C3CC3)ncc12 ; CNc1ncc(-c2nc3cc(C4CCN([C@]5(C)COC[C@@H]5O)CC4)ccc3o2)c2cc(NC(=O)C3CC3)ncc12]

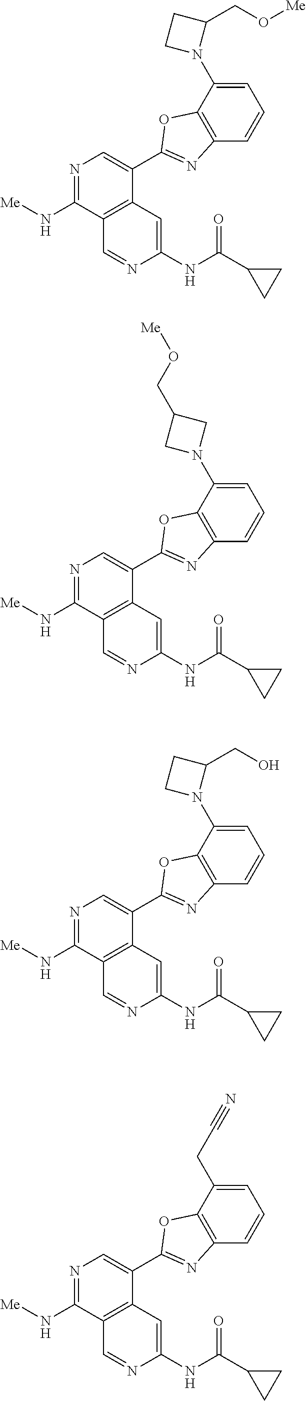 [SMILES: CNc1ncc(-c2nc3cccc(N4CCC4COC)c3o2)c2cc(NC(=O)C3CC3)ncc12 ; CNc1ncc(-c2nc3cccc(N4CC(COC)C4)c3o2)c2cc(NC(=O)C3CC3)ncc12 ; CNc1ncc(-c2nc3cccc(N4CCC4CO)c3o2)c2cc(NC(=O)C3CC3)ncc12 ; CNc1ncc(-c2nc3cccc(CC#N)c3o2)c2cc(NC(=O)C3CC3)ncc12]

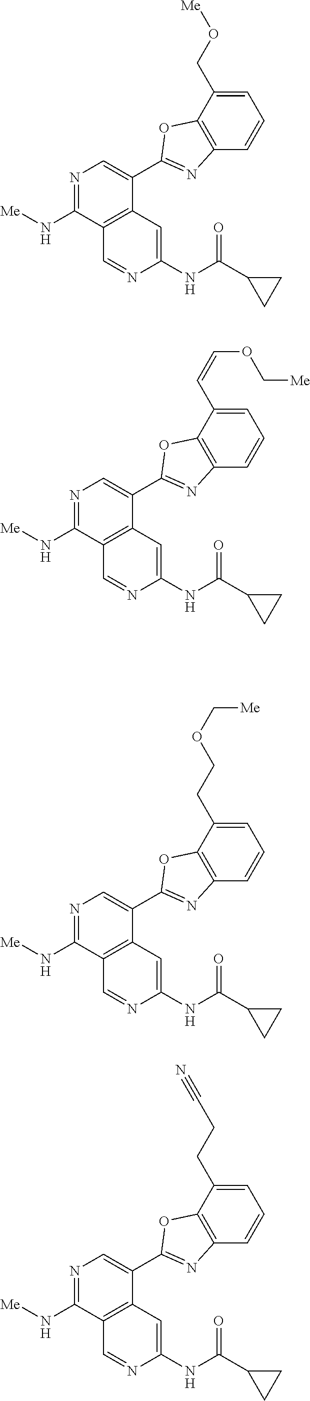 [SMILES: CNc1ncc(-c2nc3cccc(COC)c3o2)c2cc(NC(=O)C3CC3)ncc12 ; CCO/C=C\c1cccc2nc(-c3cnc(NC)c4cnc(NC(=O)C5CC5)cc34)oc12 ; CCOCCc1cccc2nc(-c3cnc(NC)c4cnc(NC(=O)C5CC5)cc34)oc12 ; CNc1ncc(-c2nc3cccc(CCC#N)c3o2)c2cc(NC(=O)C3CC3)ncc12]

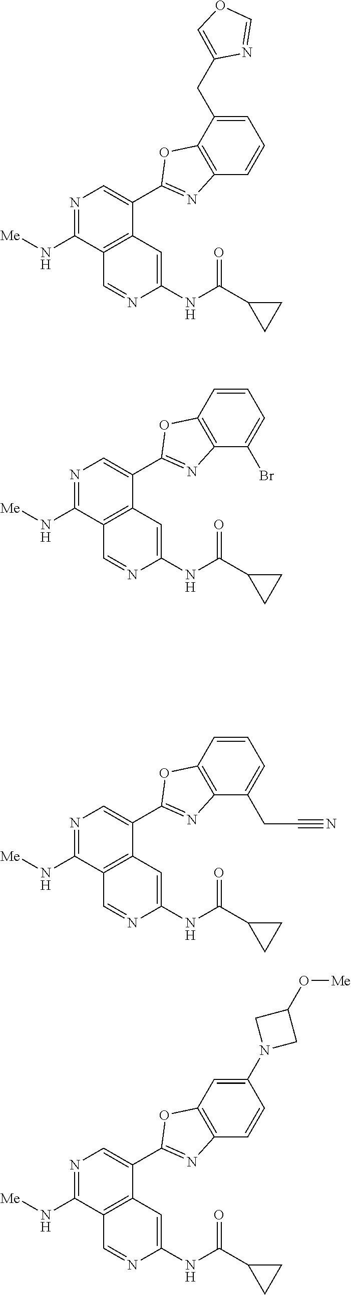 [SMILES: CNc1ncc(-c2nc3cccc(Cc4cocn4)c3o2)c2cc(NC(=O)C3CC3)ncc12 ; CNc1ncc(-c2nc3c(Br)cccc3o2)c2cc(NC(=O)C3CC3)ncc12 ; CNc1ncc(-c2nc3c(CC#N)cccc3o2)c2cc(NC(=O)C3CC3)ncc12 ; CNc1ncc(-c2nc3ccc(N4CC(OC)C4)cc3o2)c2cc(NC(=O)C3CC3)ncc12]

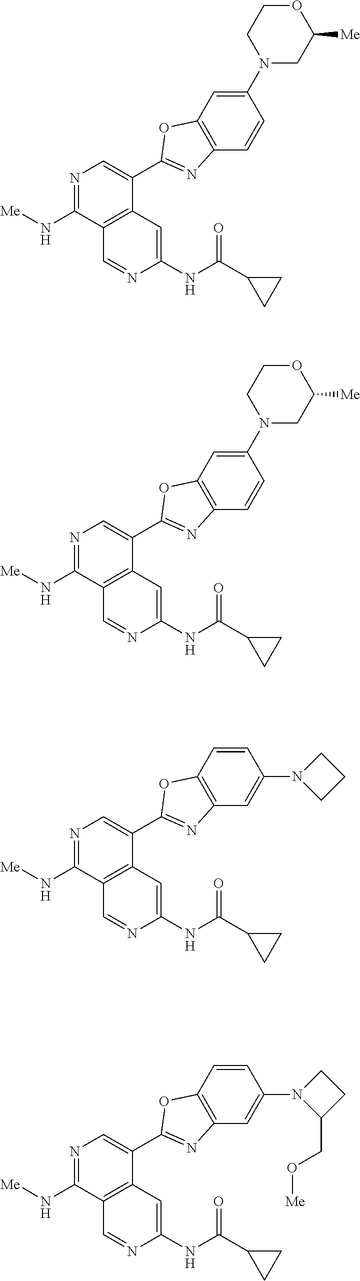 [SMILES: CNc1ncc(-c2nc3ccc(N4CCO[C@@H](C)C4)cc3o2)c2cc(NC(=O)C3CC3)ncc12 ; CNc1ncc(-c2nc3ccc(N4CCO[C@H](C)C4)cc3o2)c2cc(NC(=O)C3CC3)ncc12 ; CNc1ncc(-c2nc3cc(N4CCC4)ccc3o2)c2cc(NC(=O)C3CC3)ncc12 ; CNc1ncc(-c2nc3cc(N4CCC4COC)ccc3o2)c2cc(NC(=O)C3CC3)ncc12]

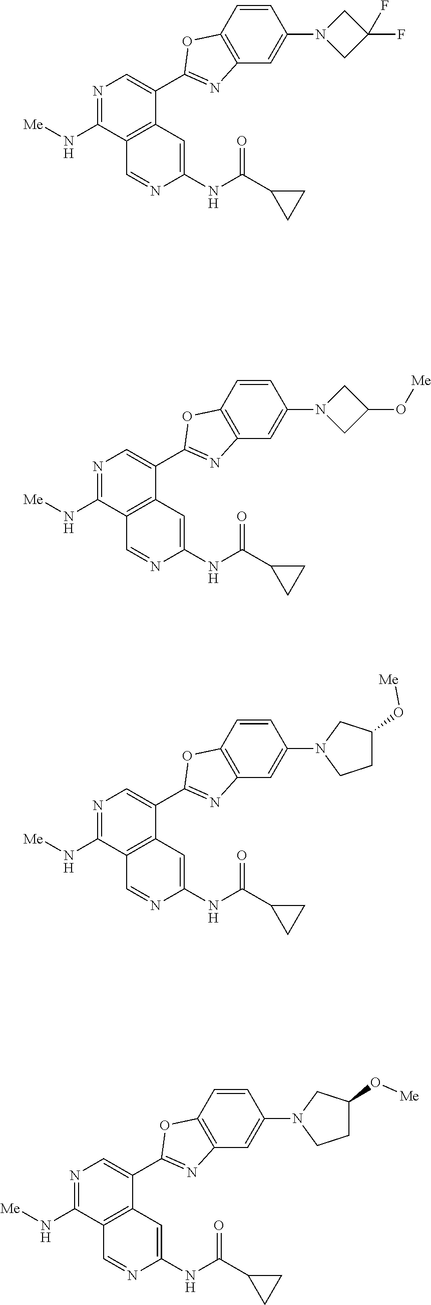 [SMILES: CNc1ncc(-c2nc3cc(N4CC(F)(F)C4)ccc3o2)c2cc(NC(=O)C3CC3)ncc12 ; CNc1ncc(-c2nc3cc(N4CC(OC)C4)ccc3o2)c2cc(NC(=O)C3CC3)ncc12 ; CNc1ncc(-c2nc3cc(N4CC[C@@H](OC)C4)ccc3o2)c2cc(NC(=O)C3CC3)ncc12 ; CNc1ncc(-c2nc3cc(N4CC[C@H](OC)C4)ccc3o2)c2cc(NC(=O)C3CC3)ncc12]

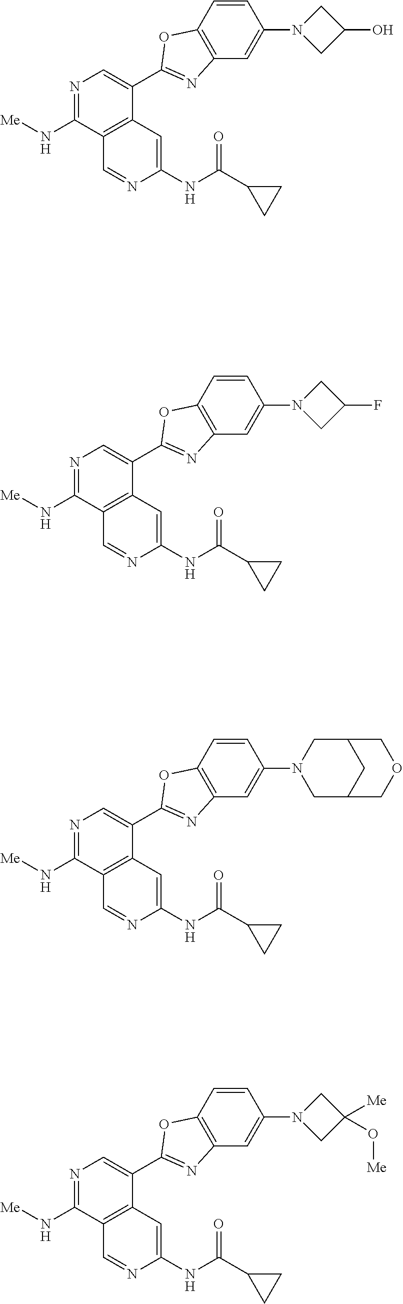 [SMILES: CNc1ncc(-c2nc3cc(N4CC(O)C4)ccc3o2)c2cc(NC(=O)C3CC3)ncc12 ; CNc1ncc(-c2nc3cc(N4CC(F)C4)ccc3o2)c2cc(NC(=O)C3CC3)ncc12 ; CNc1ncc(-c2nc3cc(N4CC5COCC(C5)C4)ccc3o2)c2cc(NC(=O)C3CC3)ncc12 ; CNc1ncc(-c2nc3cc(N4CC(C)(OC)C4)ccc3o2)c2cc(NC(=O)C3CC3)ncc12]

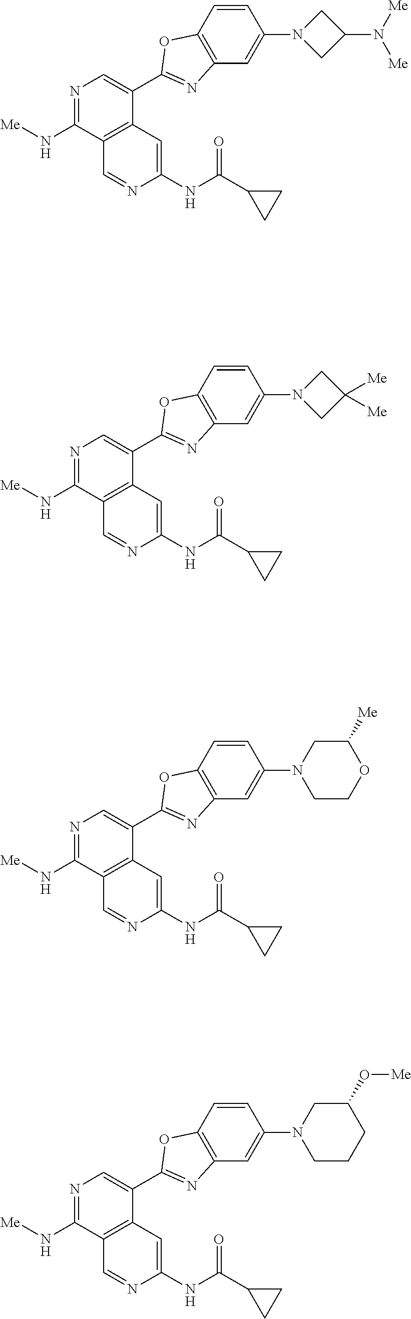 [SMILES: CNc1ncc(-c2nc3cc(N4CC(N(C)C)C4)ccc3o2)c2cc(NC(=O)C3CC3)ncc12 ; CNc1ncc(-c2nc3cc(N4CC(C)(C)C4)ccc3o2)c2cc(NC(=O)C3CC3)ncc12 ; CNc1ncc(-c2nc3cc(N4CCO[C@@H](C)C4)ccc3o2)c2cc(NC(=O)C3CC3)ncc12 ; CNc1ncc(-c2nc3cc(N4CCC[C@@H](OC)C4)ccc3o2)c2cc(NC(=O)C3CC3)ncc12]

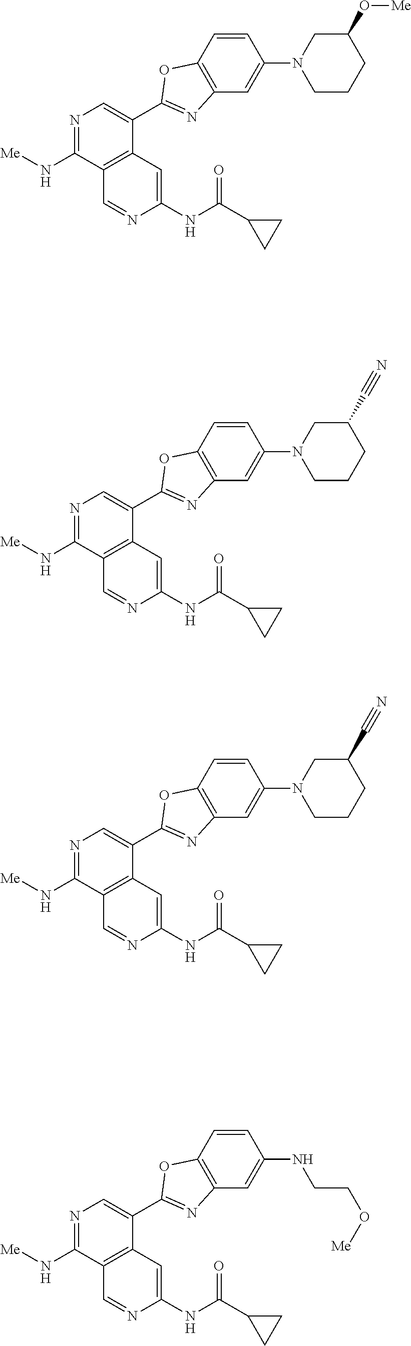 [SMILES: CNc1ncc(-c2nc3cc(N4CCC[C@H](OC)C4)ccc3o2)c2cc(NC(=O)C3CC3)ncc12 ; CNc1ncc(-c2nc3cc(N4CCC[C@@H](C#N)C4)ccc3o2)c2cc(NC(=O)C3CC3)ncc12 ; CNc1ncc(-c2nc3cc(N4CCC[C@H](C#N)C4)ccc3o2)c2cc(NC(=O)C3CC3)ncc12 ; CNc1ncc(-c2nc3cc(NCCOC)ccc3o2)c2cc(NC(=O)C3CC3)ncc12]

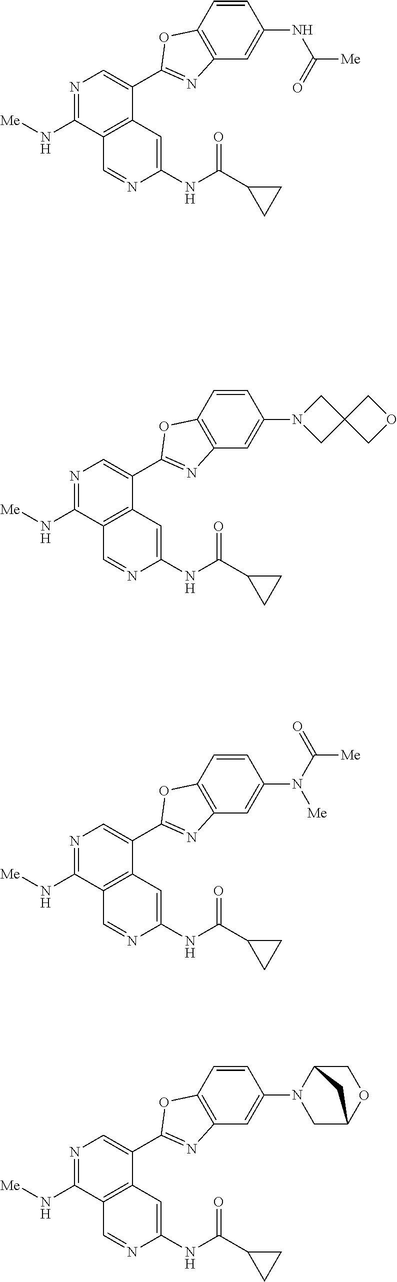 [SMILES: CNc1ncc(-c2nc3cc(NC(C)=O)ccc3o2)c2cc(NC(=O)C3CC3)ncc12 ; CNc1ncc(-c2nc3cc(N4CC5(COC5)C4)ccc3o2)c2cc(NC(=O)C3CC3)ncc12 ; CNc1ncc(-c2nc3cc(N(C)C(C)=O)ccc3o2)c2cc(NC(=O)C3CC3)ncc12 ; CNc1ncc(-c2nc3cc(N4C[C@@H]5C[C@H]4CO5)ccc3o2)c2cc(NC(=O)C3CC3)ncc12]

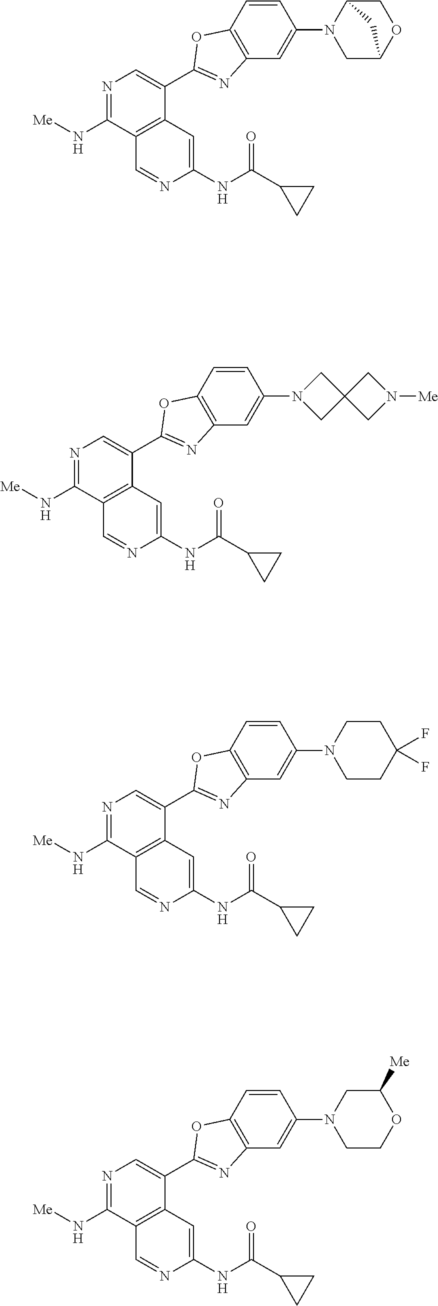 [SMILES: CNc1ncc(-c2nc3cc(N4C[C@H]5C[C@@H]4CO5)ccc3o2)c2cc(NC(=O)C3CC3)ncc12 ; CNc1ncc(-c2nc3cc(N4CC5(CN(C)C5)C4)ccc3o2)c2cc(NC(=O)C3CC3)ncc12 ; CNc1ncc(-c2nc3cc(N4CCC(F)(F)CC4)ccc3o2)c2cc(NC(=O)C3CC3)ncc12 ; CNc1ncc(-c2nc3cc(N4CCO[C@H](C)C4)ccc3o2)c2cc(NC(=O)C3CC3)ncc12]

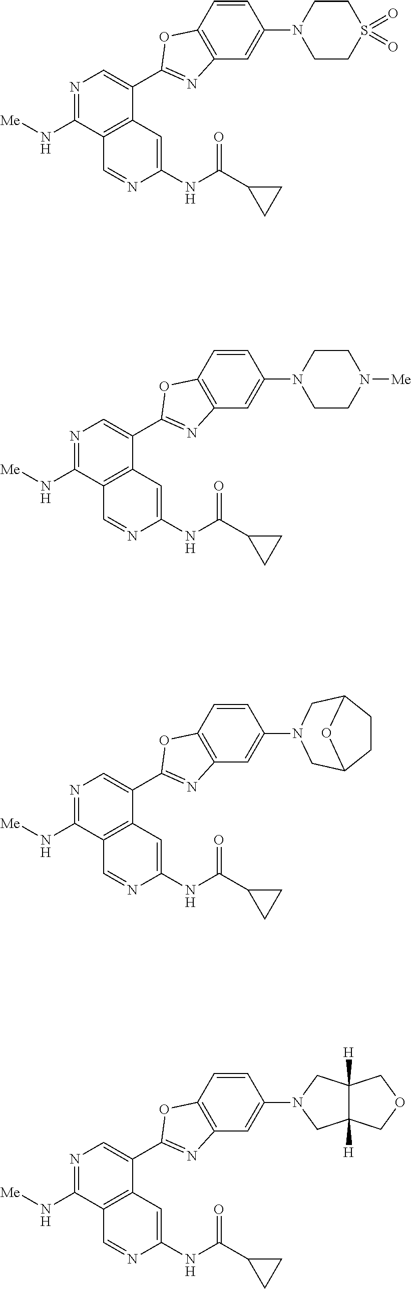 [SMILES: CNc1ncc(-c2nc3cc(N4CCS(=O)(=O)CC4)ccc3o2)c2cc(NC(=O)C3CC3)ncc12 ; CNc1ncc(-c2nc3cc(N4CCN(C)CC4)ccc3o2)c2cc(NC(=O)C3CC3)ncc12 ; CNc1ncc(-c2nc3cc(N4CC5CCC(C4)O5)ccc3o2)c2cc(NC(=O)C3CC3)ncc12 ; CNc1ncc(-c2nc3cc(N4C[C@H]5COC[C@H]5C4)ccc3o2)c2cc(NC(=O)C3CC3)ncc12]

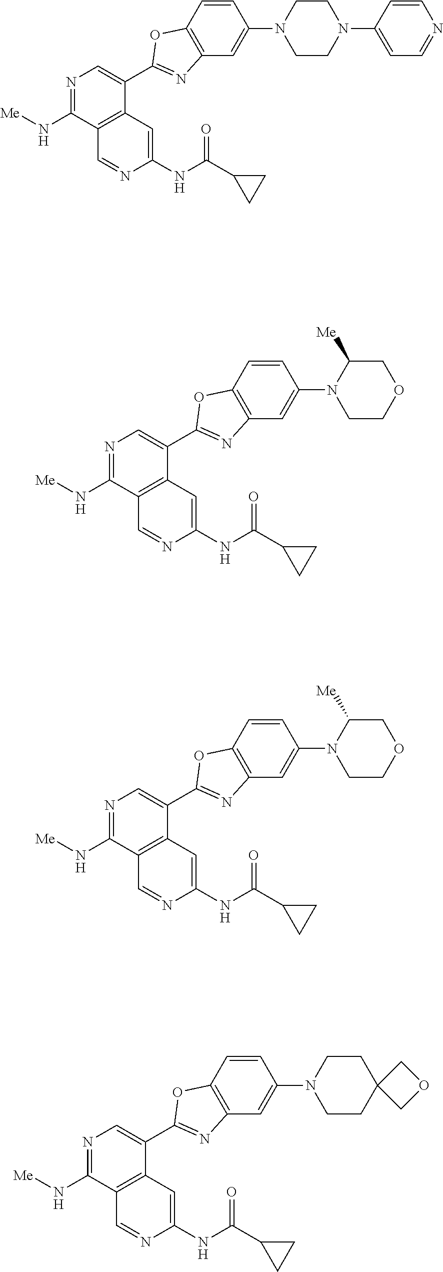 [SMILES: CNc1ncc(-c2nc3cc(N4CCN(c5ccncc5)CC4)ccc3o2)c2cc(NC(=O)C3CC3)ncc12 ; CNc1ncc(-c2nc3cc(N4CCOC[C@@H]4C)ccc3o2)c2cc(NC(=O)C3CC3)ncc12 ; CNc1ncc(-c2nc3cc(N4CCOC[C@H]4C)ccc3o2)c2cc(NC(=O)C3CC3)ncc12 ; CNc1ncc(-c2nc3cc(N4CCC5(CC4)COC5)ccc3o2)c2cc(NC(=O)C3CC3)ncc12]

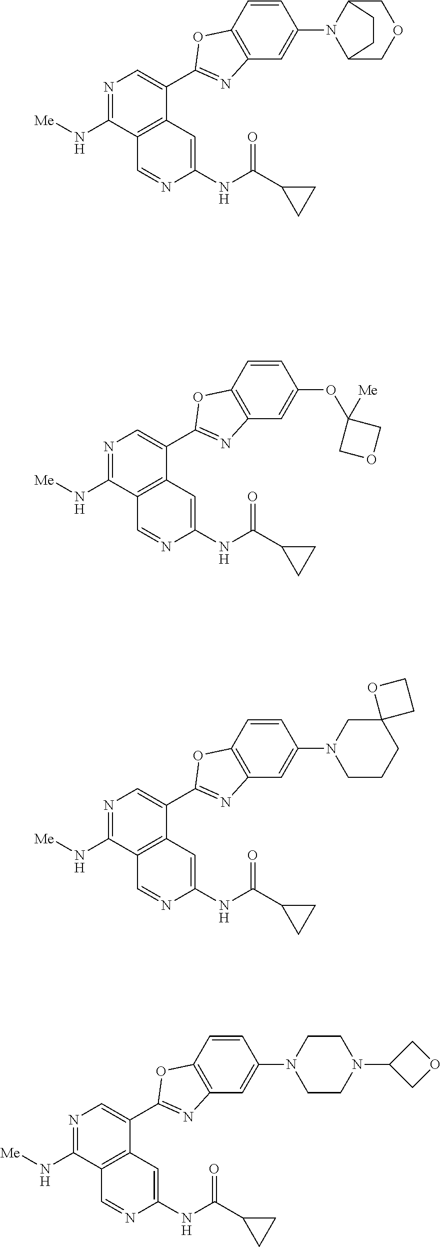 [SMILES: CNc1ncc(-c2nc3cc(N4C5CCC4COC5)ccc3o2)c2cc(NC(=O)C3CC3)ncc12 ; CNc1ncc(-c2nc3cc(OC4(C)COC4)ccc3o2)c2cc(NC(=O)C3CC3)ncc12 ; CNc1ncc(-c2nc3cc(N4CCCC5(CCO5)C4)ccc3o2)c2cc(NC(=O)C3CC3)ncc12 ; CNc1ncc(-c2nc3cc(N4CCN(C5COC5)CC4)ccc3o2)c2cc(NC(=O)C3CC3)ncc12]

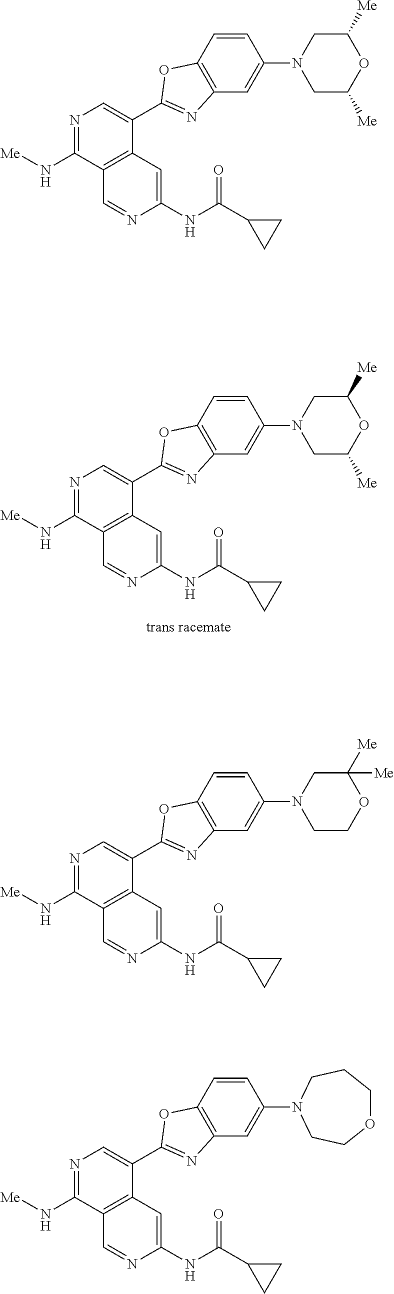 [SMILES: CNc1ncc(-c2nc3cc(N4C[C@@H](C)O[C@@H](C)C4)ccc3o2)c2cc(NC(=O)C3CC3)ncc12 ; CNc1ncc(-c2nc3cc(N4C[C@@H](C)O[C@H](C)C4)ccc3o2)c2cc(NC(=O)C3CC3)ncc12 ; CNc1ncc(-c2nc3cc(N4CCOC(C)(C)C4)ccc3o2)c2cc(NC(=O)C3CC3)ncc12 ; CNc1ncc(-c2nc3cc(N4CCCOCC4)ccc3o2)c2cc(NC(=O)C3CC3)ncc12]

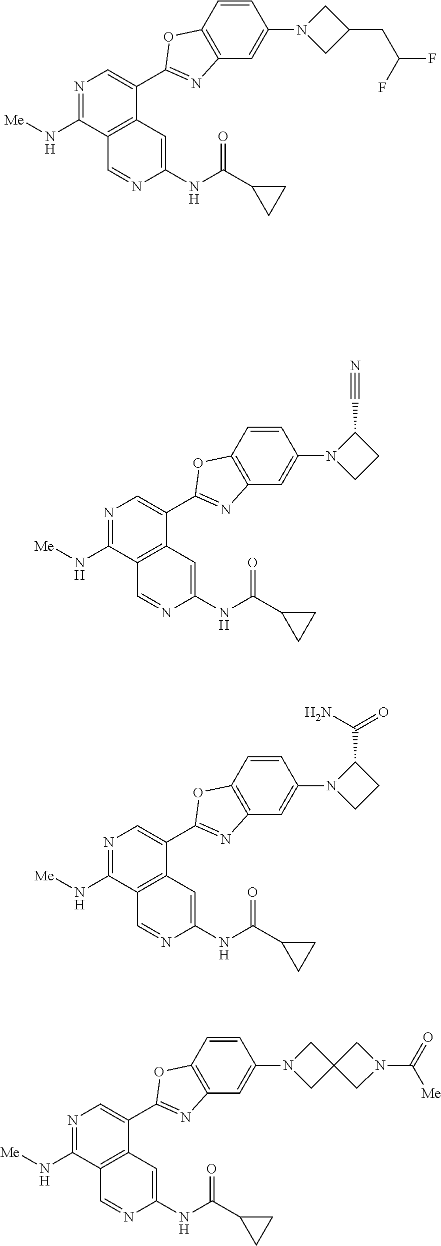 [SMILES: CNc1ncc(-c2nc3cc(N4CC(CC(F)F)C4)ccc3o2)c2cc(NC(=O)C3CC3)ncc12 ; CNc1ncc(-c2nc3cc(N4CC[C@H]4C#N)ccc3o2)c2cc(NC(=O)C3CC3)ncc12 ; CNc1ncc(-c2nc3cc(N4CC[C@H]4C(N)=O)ccc3o2)c2cc(NC(=O)C3CC3)ncc12 ; CNc1ncc(-c2nc3cc(N4CC5(CN(C(C)=O)C5)C4)ccc3o2)c2cc(NC(=O)C3CC3)ncc12]

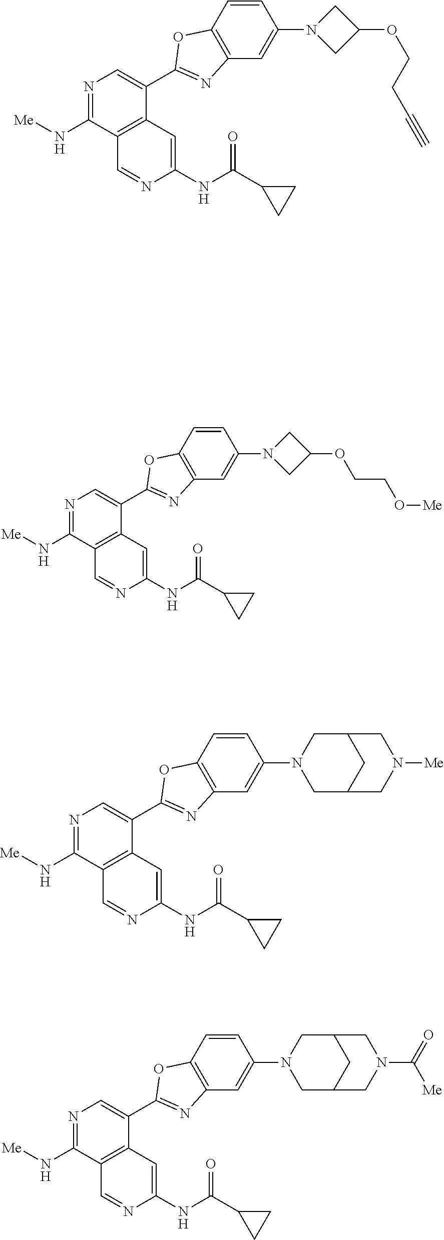 [SMILES: C#CCCOC1CN(c2ccc3oc(-c4cnc(NC)c5cnc(NC(=O)C6CC6)cc45)nc3c2)C1 ; CNc1ncc(-c2nc3cc(N4CC(OCCOC)C4)ccc3o2)c2cc(NC(=O)C3CC3)ncc12 ; CNc1ncc(-c2nc3cc(N4CC5CC(CN(C)C5)C4)ccc3o2)c2cc(NC(=O)C3CC3)ncc12 ; CNc1ncc(-c2nc3cc(N4CC5CC(CN(C(C)=O)C5)C4)ccc3o2)c2cc(NC(=O)C3CC3)ncc12]

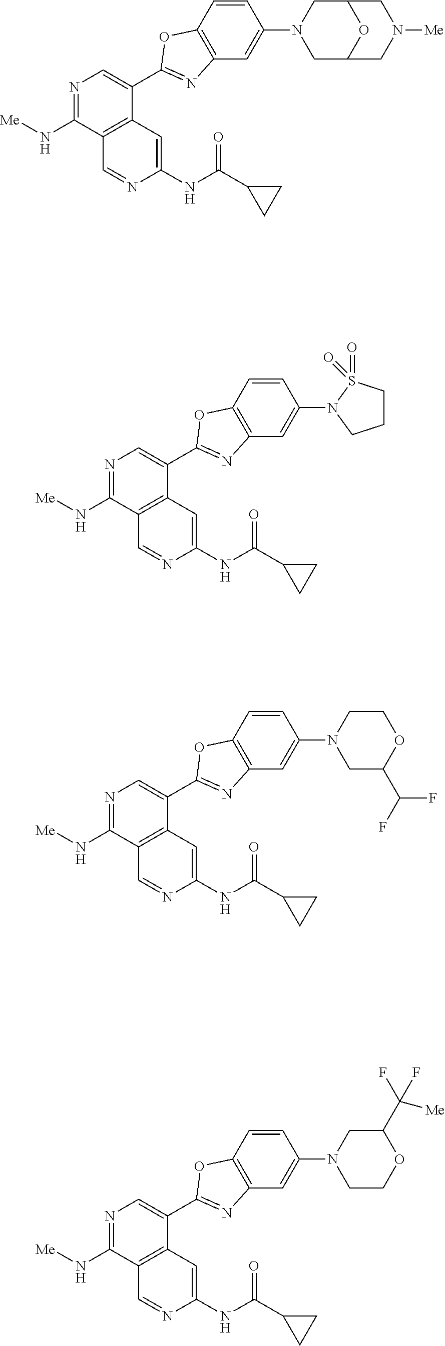 [SMILES: CNc1ncc(-c2nc3cc(N4CC5CN(C)CC(C4)O5)ccc3o2)c2cc(NC(=O)C3CC3)ncc12 ; CNc1ncc(-c2nc3cc(N4CCCS4(=O)=O)ccc3o2)c2cc(NC(=O)C3CC3)ncc12 ; CNc1ncc(-c2nc3cc(N4CCOC(C(F)F)C4)ccc3o2)c2cc(NC(=O)C3CC3)ncc12 ; CNc1ncc(-c2nc3cc(N4CCOC(C(C)(F)F)C4)ccc3o2)c2cc(NC(=O)C3CC3)ncc12]

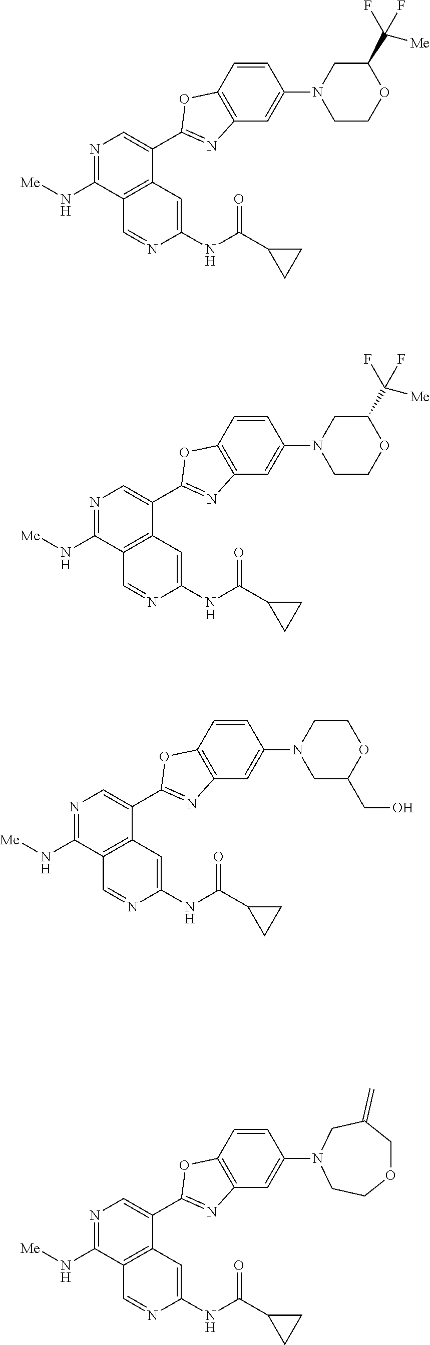 [SMILES: CNc1ncc(-c2nc3cc(N4CCO[C@H](C(C)(F)F)C4)ccc3o2)c2cc(NC(=O)C3CC3)ncc12 ; CNc1ncc(-c2nc3cc(N4CCO[C@@H](C(C)(F)F)C4)ccc3o2)c2cc(NC(=O)C3CC3)ncc12 ; CNc1ncc(-c2nc3cc(N4CCOC(CO)C4)ccc3o2)c2cc(NC(=O)C3CC3)ncc12 ; C=C1COCCN(c2ccc3oc(-c4cnc(NC)c5cnc(NC(=O)C6CC6)cc45)nc3c2)C1]

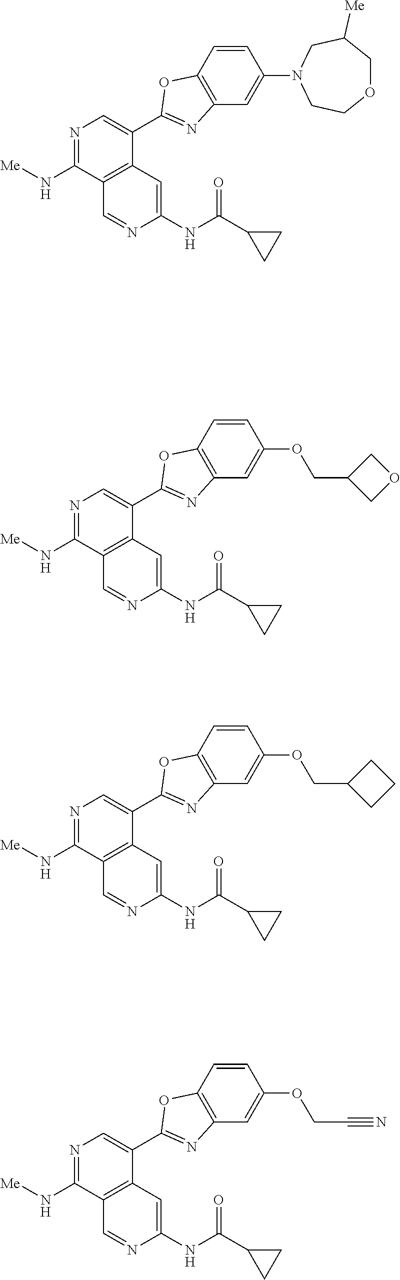 [SMILES: CNc1ncc(-c2nc3cc(N4CCOCC(C)C4)ccc3o2)c2cc(NC(=O)C3CC3)ncc12 ; CNc1ncc(-c2nc3cc(OCC4COC4)ccc3o2)c2cc(NC(=O)C3CC3)ncc12 ; CNc1ncc(-c2nc3cc(OCC4CCC4)ccc3o2)c2cc(NC(=O)C3CC3)ncc12 ; CNc1ncc(-c2nc3cc(OCC#N)ccc3o2)c2cc(NC(=O)C3CC3)ncc12]

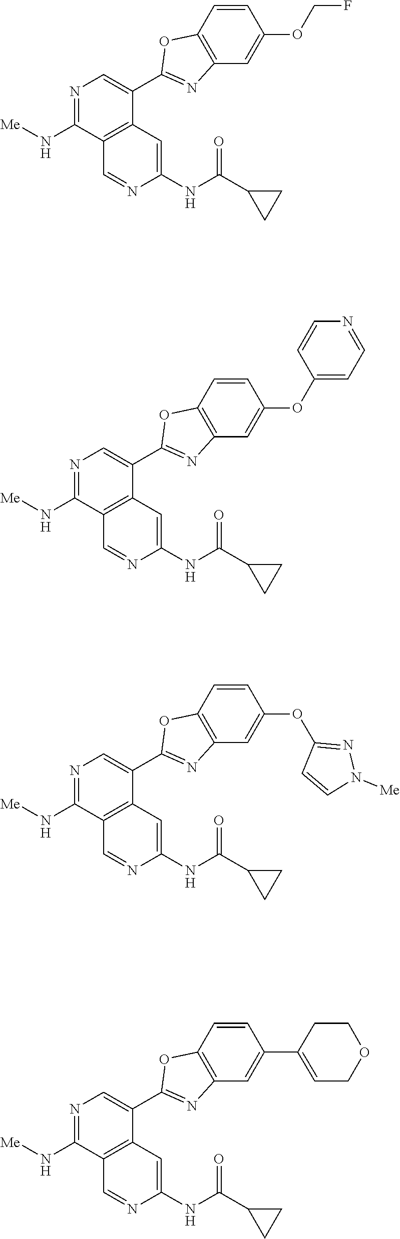 [SMILES: CNc1ncc(-c2nc3cc(OCF)ccc3o2)c2cc(NC(=O)C3CC3)ncc12 ; CNc1ncc(-c2nc3cc(Oc4ccncc4)ccc3o2)c2cc(NC(=O)C3CC3)ncc12 ; CNc1ncc(-c2nc3cc(Oc4ccn(C)n4)ccc3o2)c2cc(NC(=O)C3CC3)ncc12 ; CNc1ncc(-c2nc3cc(C4=CCOCC4)ccc3o2)c2cc(NC(=O)C3CC3)ncc12]

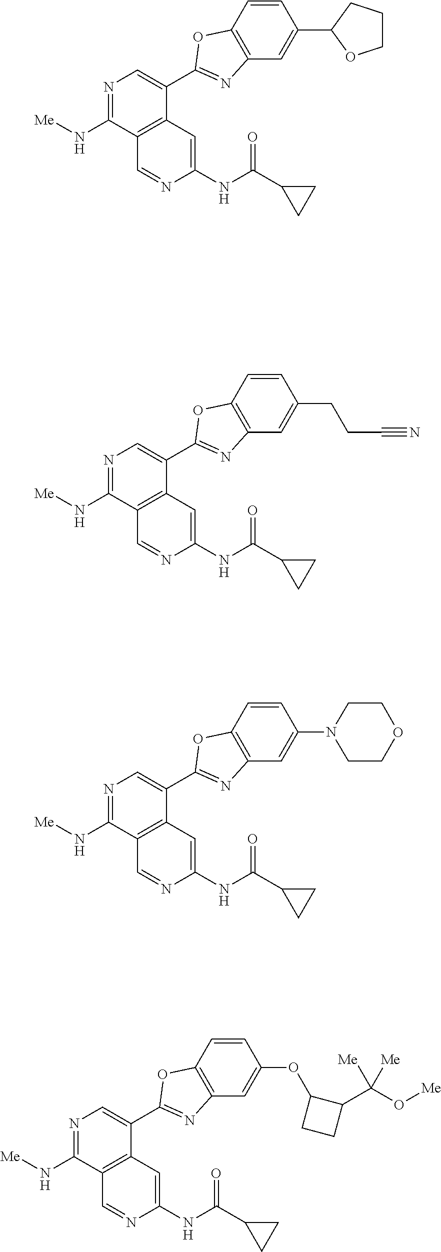 [SMILES: CNc1ncc(-c2nc3cc(C4CCCO4)ccc3o2)c2cc(NC(=O)C3CC3)ncc12 ; CNc1ncc(-c2nc3cc(CCC#N)ccc3o2)c2cc(NC(=O)C3CC3)ncc12 ; CNc1ncc(-c2nc3cc(N4CCOCC4)ccc3o2)c2cc(NC(=O)C3CC3)ncc12 ; CNc1ncc(-c2nc3cc(OC4CCC4C(C)(C)OC)ccc3o2)c2cc(NC(=O)C3CC3)ncc12]

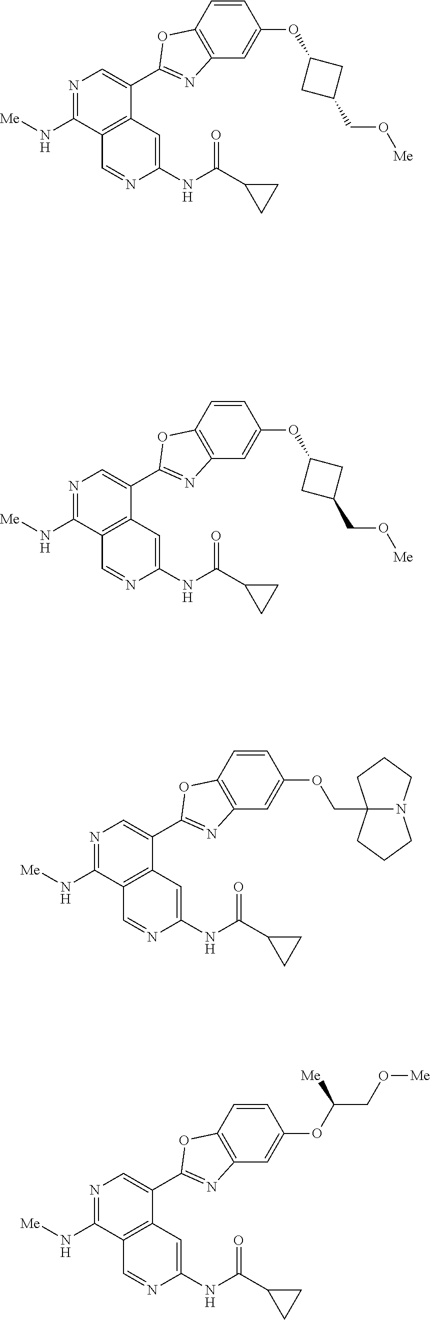 [SMILES: CNc1ncc(-c2nc3cc(O[C@H]4C[C@@H](COC)C4)ccc3o2)c2cc(NC(=O)C3CC3)ncc12 ; CNc1ncc(-c2nc3cc(O[C@H]4C[C@H](COC)C4)ccc3o2)c2cc(NC(=O)C3CC3)ncc12 ; CNc1ncc(-c2nc3cc(OCC45CCCN4CCC5)ccc3o2)c2cc(NC(=O)C3CC3)ncc12 ; CNc1ncc(-c2nc3cc(O[C@@H](C)COC)ccc3o2)c2cc(NC(=O)C3CC3)ncc12]

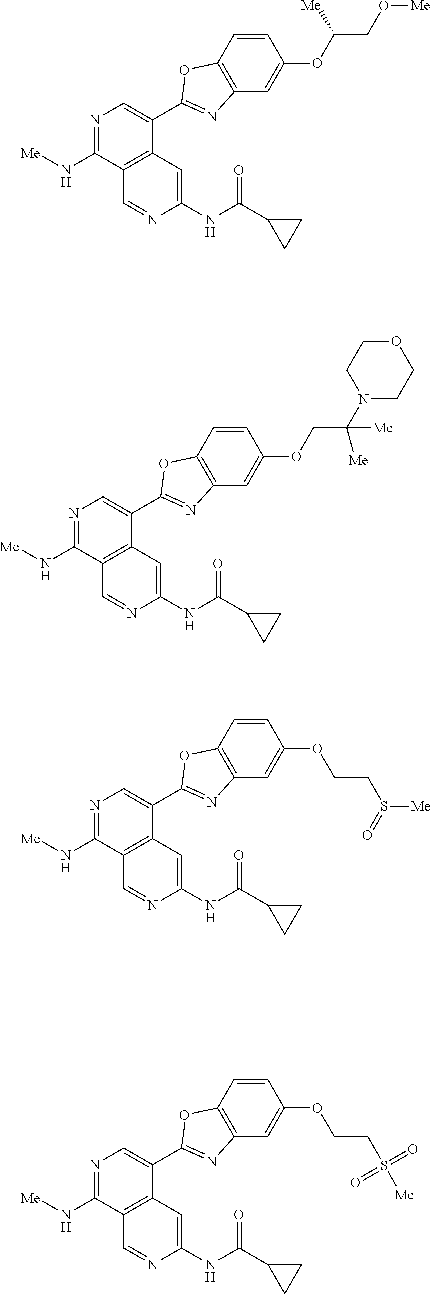 [SMILES: CNc1ncc(-c2nc3cc(O[C@H](C)COC)ccc3o2)c2cc(NC(=O)C3CC3)ncc12 ; CNc1ncc(-c2nc3cc(OCC(C)(C)N4CCOCC4)ccc3o2)c2cc(NC(=O)C3CC3)ncc12 ; CNc1ncc(-c2nc3cc(OCCS(C)=O)ccc3o2)c2cc(NC(=O)C3CC3)ncc12 ; CNc1ncc(-c2nc3cc(OCCS(C)(=O)=O)ccc3o2)c2cc(NC(=O)C3CC3)ncc12]

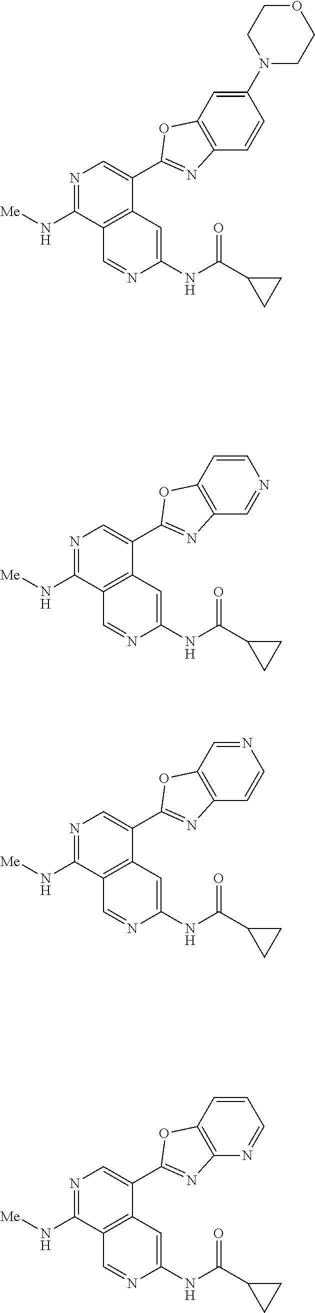 [SMILES: CNc1ncc(-c2nc3ccc(N4CCOCC4)cc3o2)c2cc(NC(=O)C3CC3)ncc12 ; CNc1ncc(-c2nc3cnccc3o2)c2cc(NC(=O)C3CC3)ncc12 ; CNc1ncc(-c2nc3ccncc3o2)c2cc(NC(=O)C3CC3)ncc12 ; CNc1ncc(-c2nc3ncccc3o2)c2cc(NC(=O)C3CC3)ncc12]

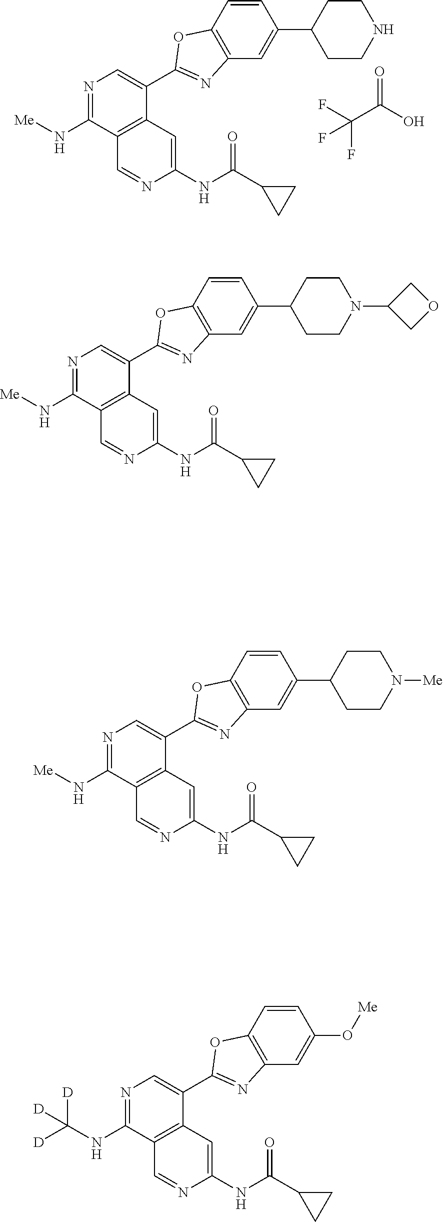

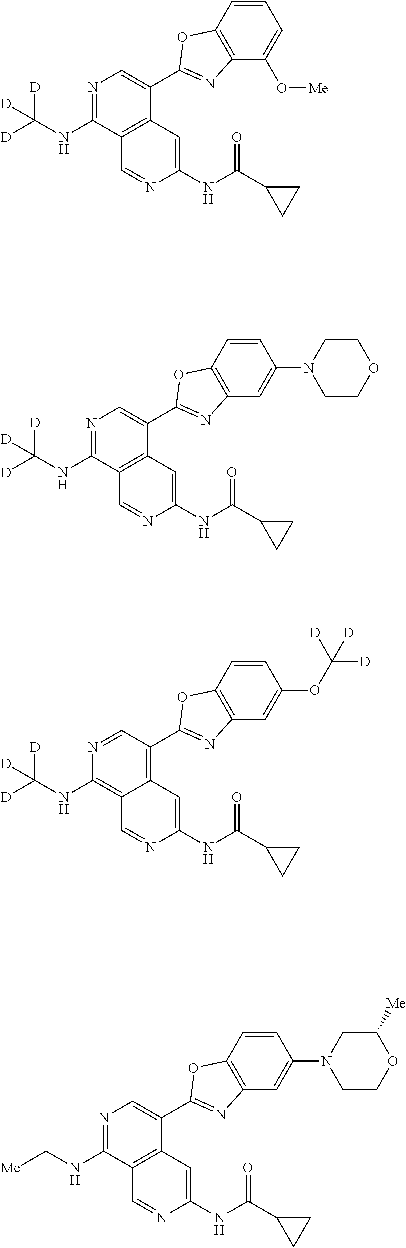 [SMILES: [2H]C([2H])([2H])Nc1ncc(-c2nc3c(OC)cccc3o2)c2cc(NC(=O)C3CC3)ncc12 ; [2H]C([2H])([2H])Nc1ncc(-c2nc3cc(N4CCOCC4)ccc3o2)c2cc(NC(=O)C3CC3)ncc12 ; [2H]C([2H])([2H])Nc1ncc(-c2nc3cc(OC([2H])([2H])[2H])ccc3o2)c2cc(NC(=O)C3CC3)ncc12 ; CCNc1ncc(-c2nc3cc(N4CCO[C@@H](C)C4)ccc3o2)c2cc(NC(=O)C3CC3)ncc12]

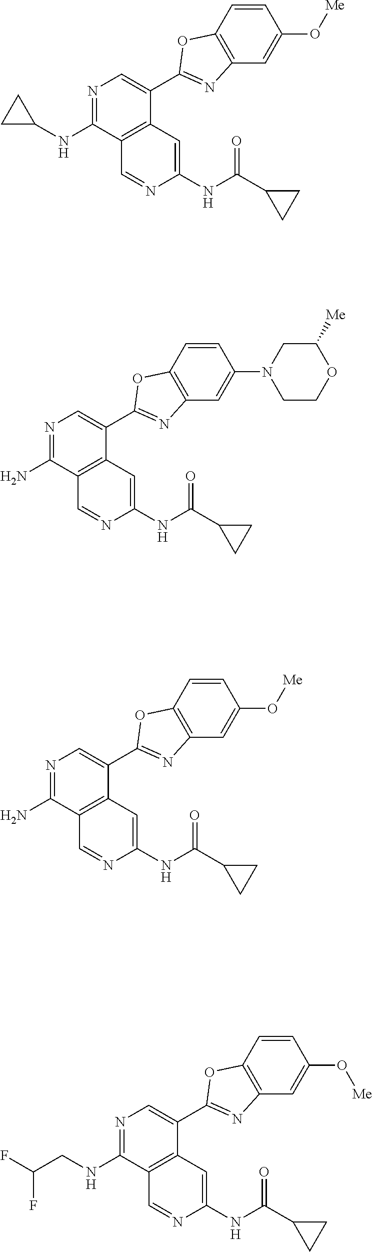 [SMILES: COc1ccc2oc(-c3cnc(NC4CC4)c4cnc(NC(=O)C5CC5)cc34)nc2c1 ; C[C@H]1CN(c2ccc3oc(-c4cnc(N)c5cnc(NC(=O)C6CC6)cc45)nc3c2)CCO1 ; COc1ccc2oc(-c3cnc(N)c4cnc(NC(=O)C5CC5)cc34)nc2c1 ; COc1ccc2oc(-c3cnc(NCC(F)F)c4cnc(NC(=O)C5CC5)cc34)nc2c1]

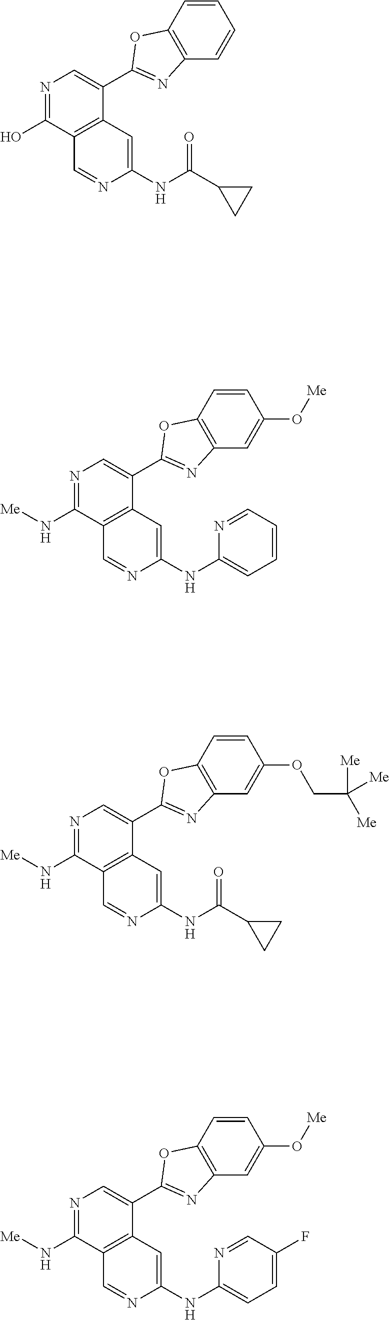 [SMILES: O=C(Nc1cc2c(-c3nc4ccccc4o3)cnc(O)c2cn1)C1CC1 ; CNc1ncc(-c2nc3cc(OC)ccc3o2)c2cc(Nc3ccccn3)ncc12 ; CNc1ncc(-c2nc3cc(OCC(C)(C)C)ccc3o2)c2cc(NC(=O)C3CC3)ncc12 ; CNc1ncc(-c2nc3cc(OC)ccc3o2)c2cc(Nc3ccc(F)cn3)ncc12]

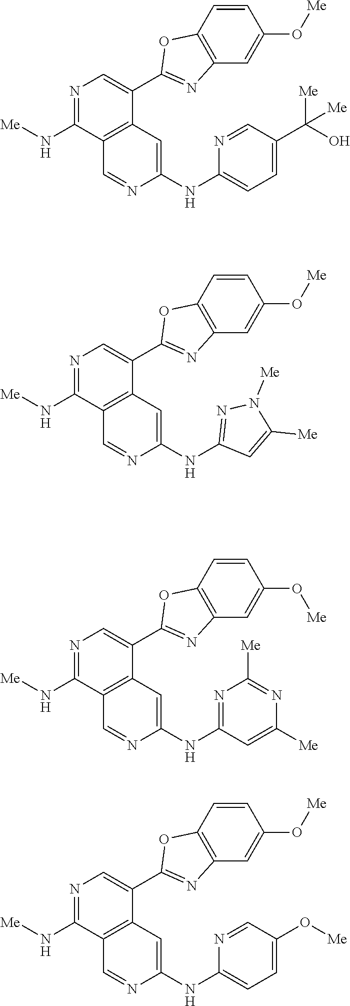 [SMILES: CNc1ncc(-c2nc3cc(OC)ccc3o2)c2cc(Nc3ccc(C(C)(C)O)cn3)ncc12 ; CNc1ncc(-c2nc3cc(OC)ccc3o2)c2cc(Nc3cc(C)n(C)n3)ncc12 ; CNc1ncc(-c2nc3cc(OC)ccc3o2)c2cc(Nc3cc(C)nc(C)n3)ncc12 ; CNc1ncc(-c2nc3cc(OC)ccc3o2)c2cc(Nc3ccc(OC)cn3)ncc12]

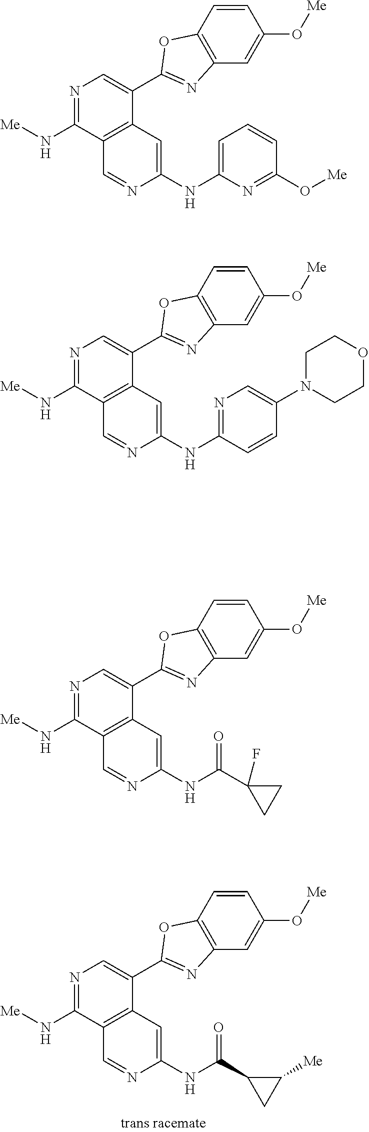 [SMILES: CNc1ncc(-c2nc3cc(OC)ccc3o2)c2cc(Nc3cccc(OC)n3)ncc12 ; CNc1ncc(-c2nc3cc(OC)ccc3o2)c2cc(Nc3ccc(N4CCOCC4)cn3)ncc12 ; CNc1ncc(-c2nc3cc(OC)ccc3o2)c2cc(NC(=O)C3(F)CC3)ncc12 ; CNc1ncc(-c2nc3cc(OC)ccc3o2)c2cc(NC(=O)[C@@H]3C[C@H]3C)ncc12]

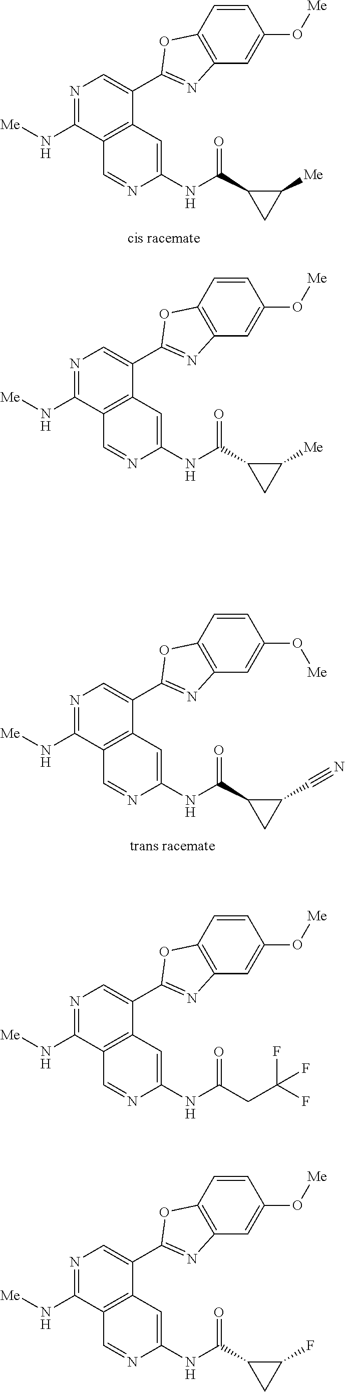 [SMILES: CNc1ncc(-c2nc3cc(OC)ccc3o2)c2cc(NC(=O)[C@@H]3C[C@@H]3C)ncc12 ; CNc1ncc(-c2nc3cc(OC)ccc3o2)c2cc(NC(=O)[C@H]3C[C@H]3C)ncc12 ; CNc1ncc(-c2nc3cc(OC)ccc3o2)c2cc(NC(=O)[C@@H]3C[C@H]3C#N)ncc12 ; CNc1ncc(-c2nc3cc(OC)ccc3o2)c2cc(NC(=O)CC(F)(F)F)ncc12 ; CNc1ncc(-c2nc3cc(OC)ccc3o2)c2cc(NC(=O)[C@H]3C[C@H]3F)ncc12]

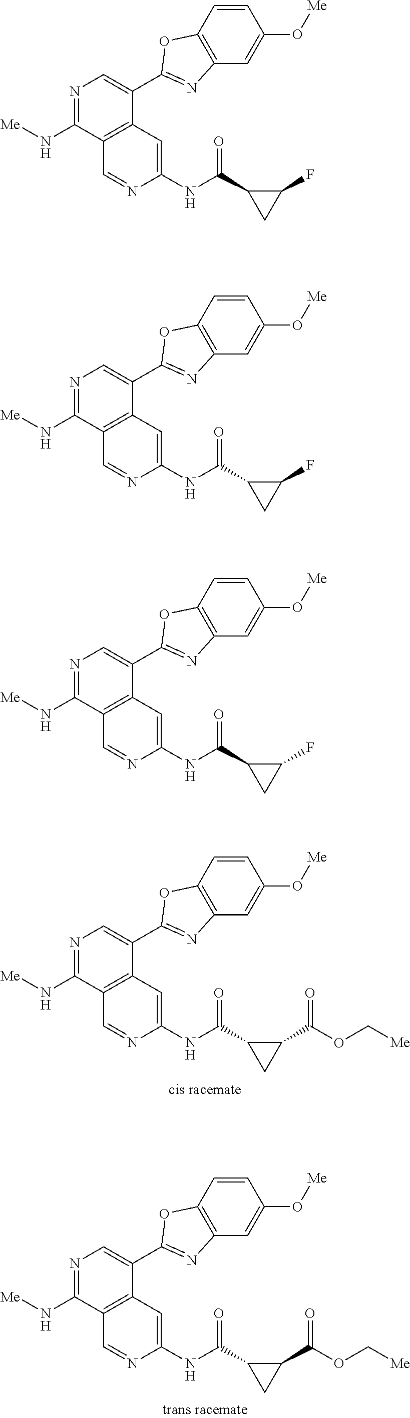 [SMILES: CNc1ncc(-c2nc3cc(OC)ccc3o2)c2cc(NC(=O)[C@@H]3C[C@@H]3F)ncc12 ; CNc1ncc(-c2nc3cc(OC)ccc3o2)c2cc(NC(=O)[C@H]3C[C@@H]3F)ncc12 ; CNc1ncc(-c2nc3cc(OC)ccc3o2)c2cc(NC(=O)[C@@H]3C[C@H]3F)ncc12 ; CCOC(=O)[C@@H]1C[C@@H]1C(=O)Nc1cc2c(-c3nc4cc(OC)ccc4o3)cnc(NC)c2cn1 ; CCOC(=O)[C@H]1C[C@@H]1C(=O)Nc1cc2c(-c3nc4cc(OC)ccc4o3)cnc(NC)c2cn1]

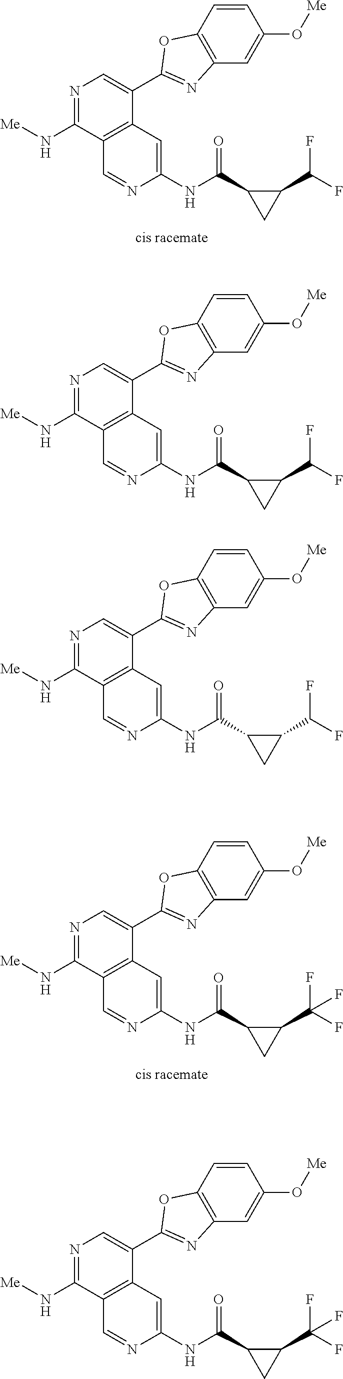

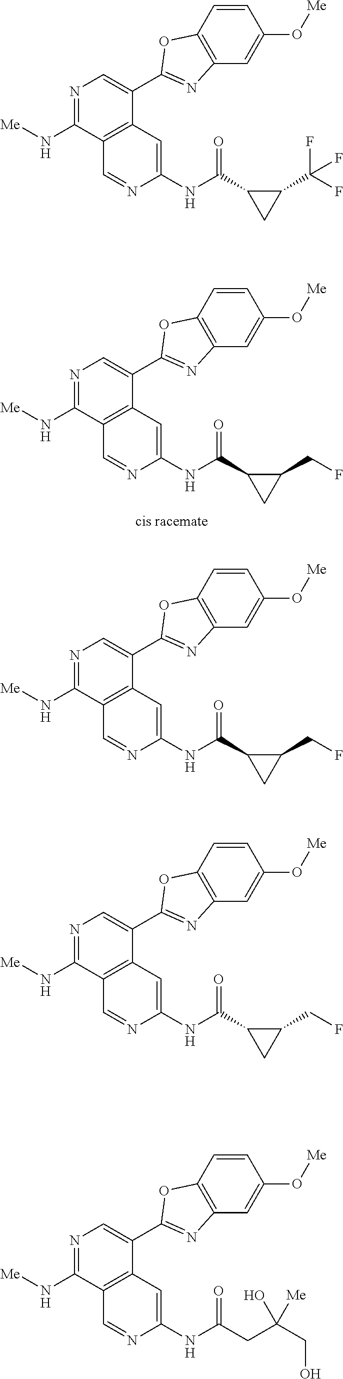 [SMILES: CNc1ncc(-c2nc3cc(OC)ccc3o2)c2cc(NC(=O)[C@H]3C[C@H]3C(F)(F)F)ncc12 ; CNc1ncc(-c2nc3cc(OC)ccc3o2)c2cc(NC(=O)[C@@H]3C[C@@H]3CF)ncc12 ; CNc1ncc(-c2nc3cc(OC)ccc3o2)c2cc(NC(=O)[C@@H]3C[C@@H]3CF)ncc12 ; CNc1ncc(-c2nc3cc(OC)ccc3o2)c2cc(NC(=O)[C@H]3C[C@H]3CF)ncc12 ; CNc1ncc(-c2nc3cc(OC)ccc3o2)c2cc(NC(=O)CC(C)(O)CO)ncc12]

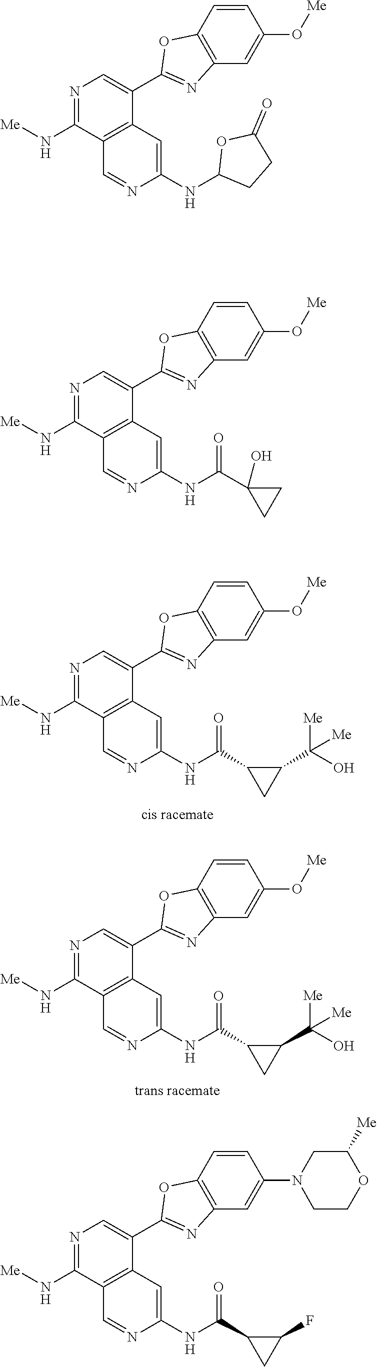 [SMILES: CNc1ncc(-c2nc3cc(OC)ccc3o2)c2cc(NC3CCC(=O)O3)ncc12 ; CNc1ncc(-c2nc3cc(OC)ccc3o2)c2cc(NC(=O)C3(O)CC3)ncc12 ; CNc1ncc(-c2nc3cc(OC)ccc3o2)c2cc(NC(=O)[C@H]3C[C@H]3C(C)(C)O)ncc12 ; CNc1ncc(-c2nc3cc(OC)ccc3o2)c2cc(NC(=O)[C@H]3C[C@@H]3C(C)(C)O)ncc12 ; CNc1ncc(-c2nc3cc(N4CCO[C@@H](C)C4)ccc3o2)c2cc(NC(=O)[C@@H]3C[C@@H]3F)ncc12]

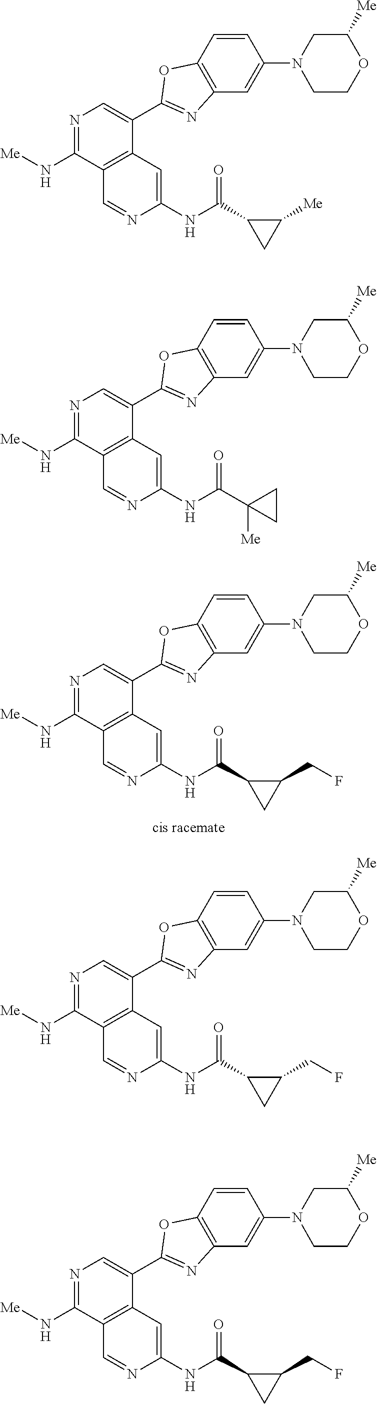 [SMILES: CNc1ncc(-c2nc3cc(N4CCO[C@@H](C)C4)ccc3o2)c2cc(NC(=O)[C@H]3C[C@H]3C)ncc12 ; CNc1ncc(-c2nc3cc(N4CCO[C@@H](C)C4)ccc3o2)c2cc(NC(=O)C3(C)CC3)ncc12 ; CNc1ncc(-c2nc3cc(N4CCO[C@@H](C)C4)ccc3o2)c2cc(NC(=O)[C@@H]3C[C@@H]3CF)ncc12 ; CNc1ncc(-c2nc3cc(N4CCO[C@@H](C)C4)ccc3o2)c2cc(NC(=O)[C@H]3C[C@H]3CF)ncc12 ; CNc1ncc(-c2nc3cc(N4CCO[C@@H](C)C4)ccc3o2)c2cc(NC(=O)[C@@H]3C[C@@H]3CF)ncc12]

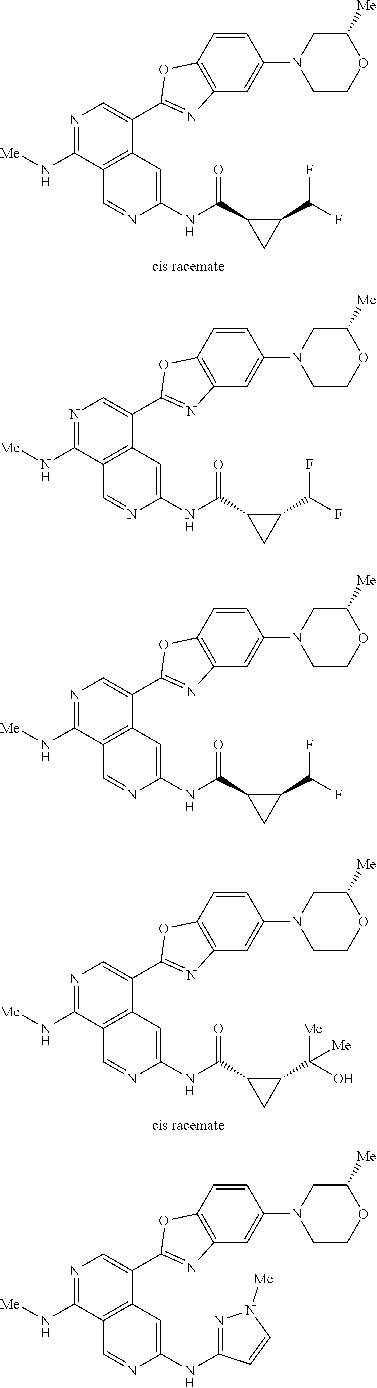 [SMILES: CNc1ncc(-c2nc3cc(N4CCO[C@@H](C)C4)ccc3o2)c2cc(NC(=O)[C@@H]3C[C@@H]3C(F)F)ncc12 ; CNc1ncc(-c2nc3cc(N4CCO[C@@H](C)C4)ccc3o2)c2cc(NC(=O)[C@H]3C[C@H]3C(F)F)ncc12 ; CNc1ncc(-c2nc3cc(N4CCO[C@@H](C)C4)ccc3o2)c2cc(NC(=O)[C@@H]3C[C@@H]3C(F)F)ncc12 ; CNc1ncc(-c2nc3cc(N4CCO[C@@H](C)C4)ccc3o2)c2cc(NC(=O)[C@H]3C[C@H]3C(C)(C)O)ncc12 ; CNc1ncc(-c2nc3cc(N4CCO[C@@H](C)C4)ccc3o2)c2cc(Nc3ccn(C)n3)ncc12]

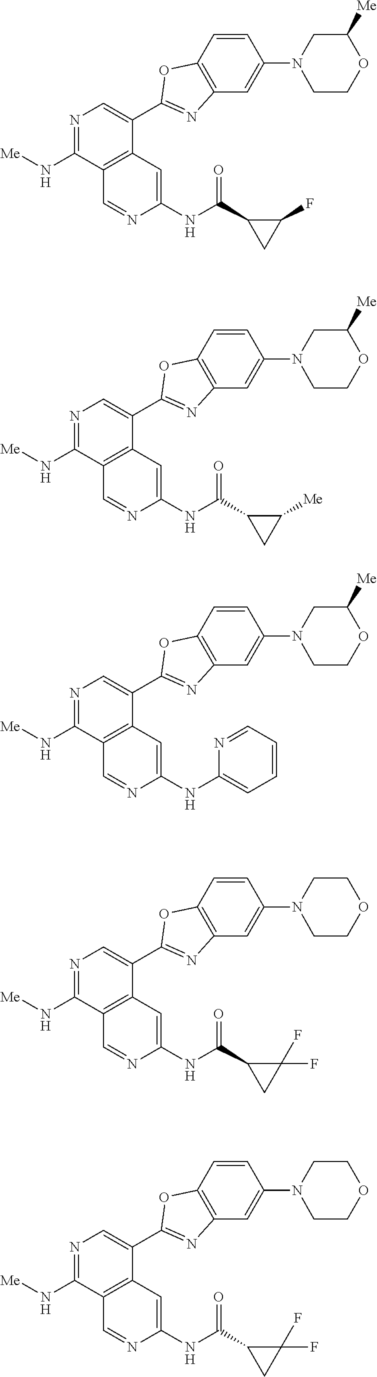 [SMILES: CNc1ncc(-c2nc3cc(N4CCO[C@H](C)C4)ccc3o2)c2cc(NC(=O)[C@@H]3C[C@@H]3F)ncc12 ; CNc1ncc(-c2nc3cc(N4CCO[C@H](C)C4)ccc3o2)c2cc(NC(=O)[C@H]3C[C@H]3C)ncc12 ; CNc1ncc(-c2nc3cc(N4CCO[C@H](C)C4)ccc3o2)c2cc(Nc3ccccn3)ncc12 ; CNc1ncc(-c2nc3cc(N4CCOCC4)ccc3o2)c2cc(NC(=O)[C@@H]3CC3(F)F)ncc12 ; CNc1ncc(-c2nc3cc(N4CCOCC4)ccc3o2)c2cc(NC(=O)[C@H]3CC3(F)F)ncc12]

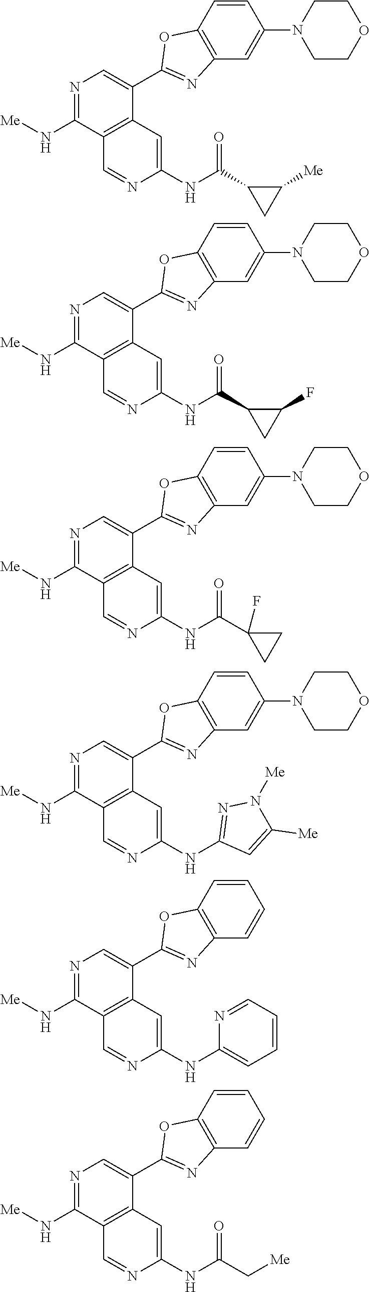

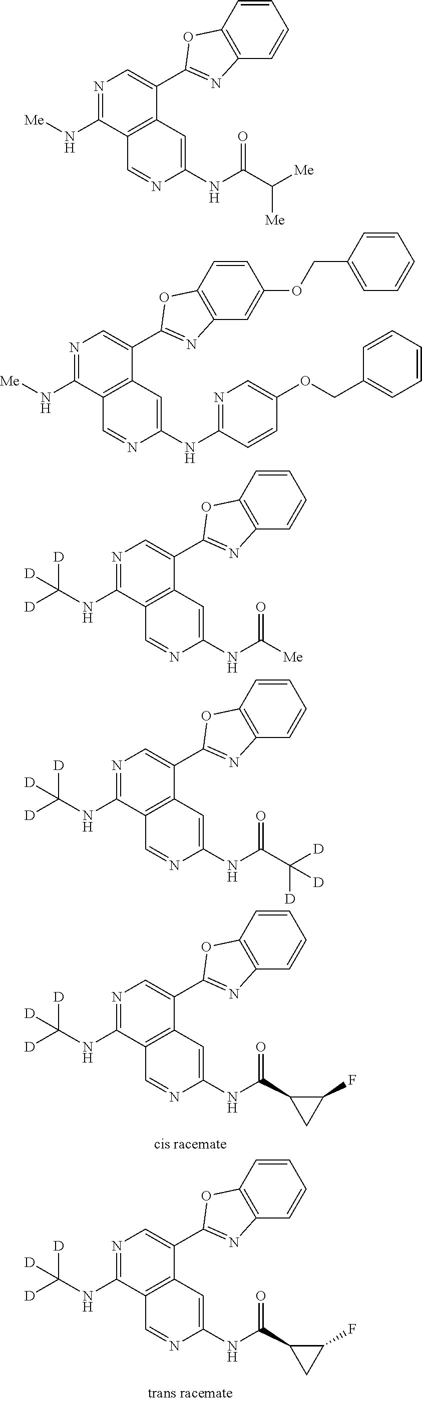

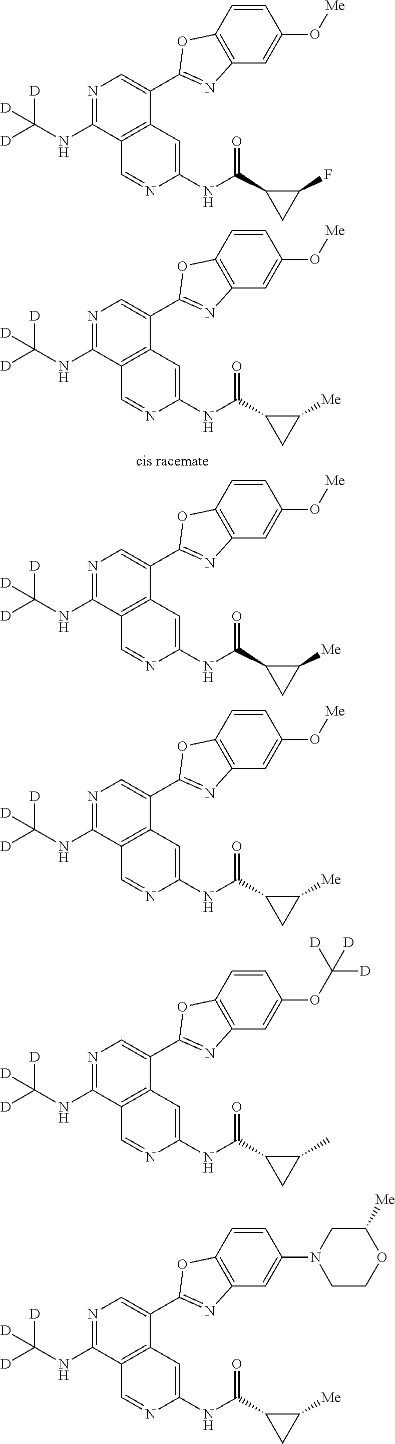

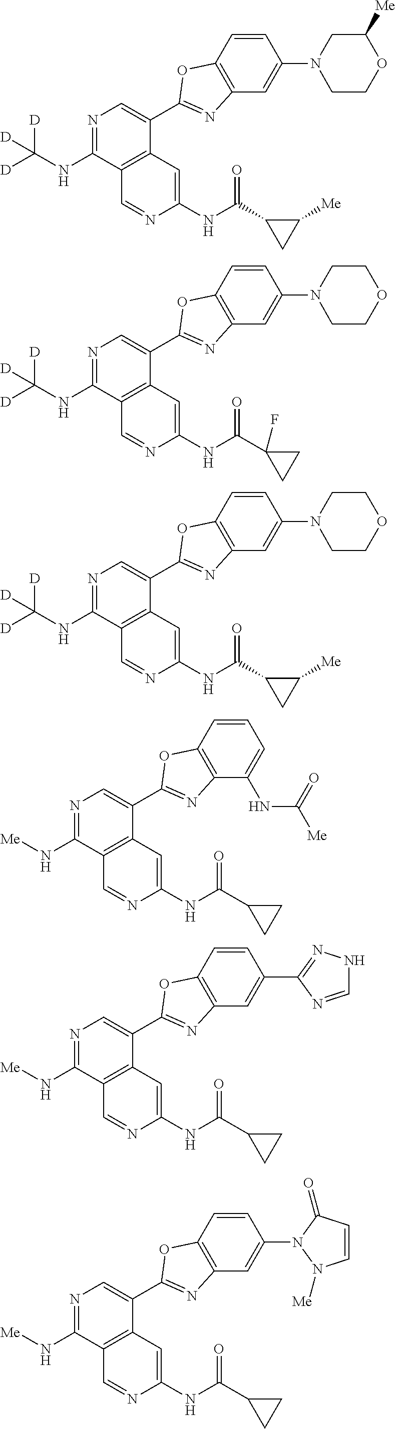

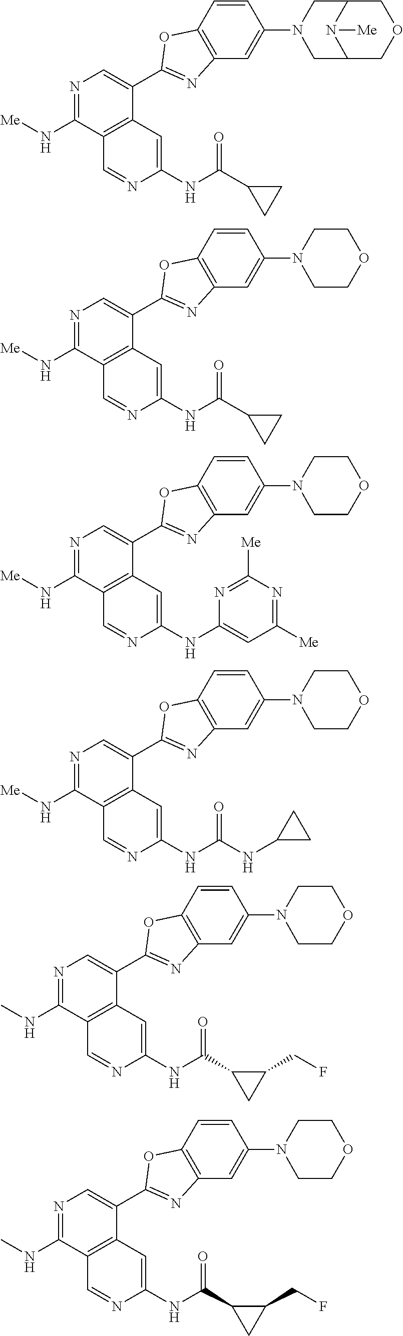

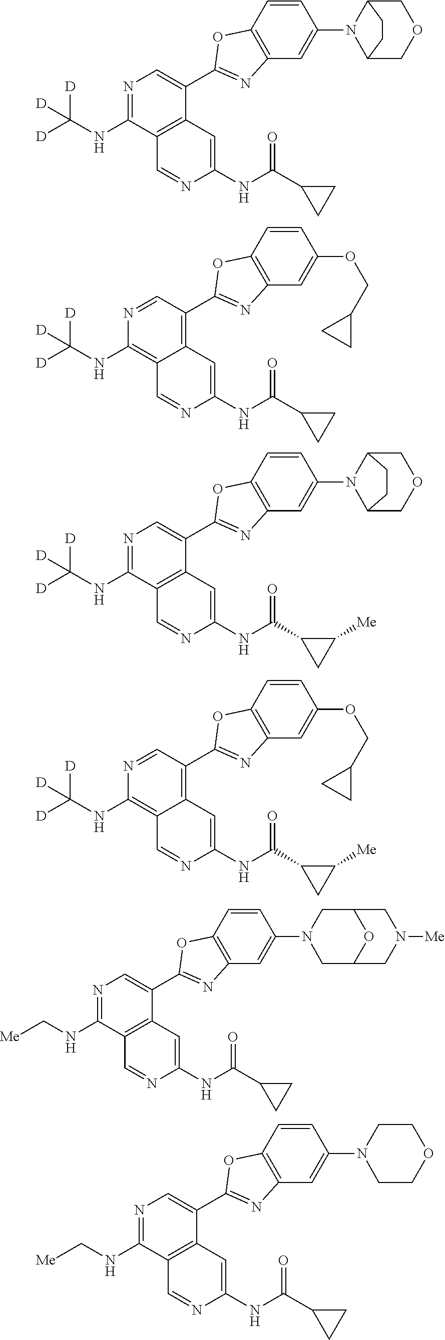

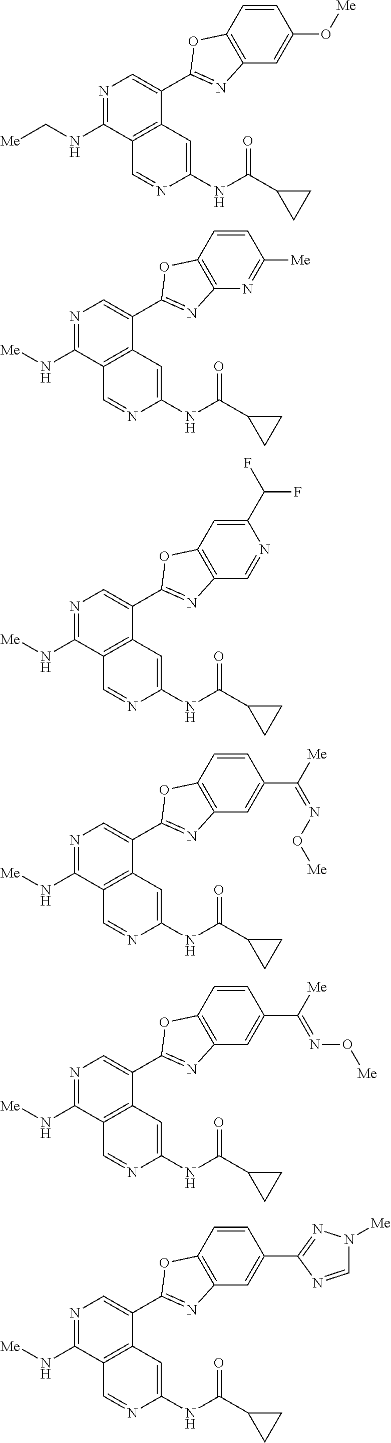

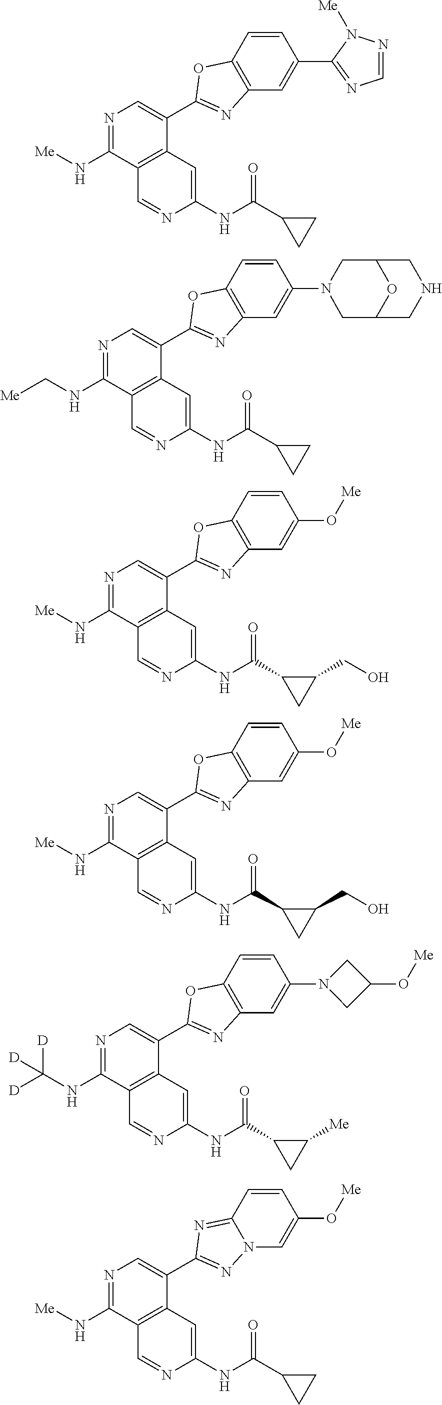

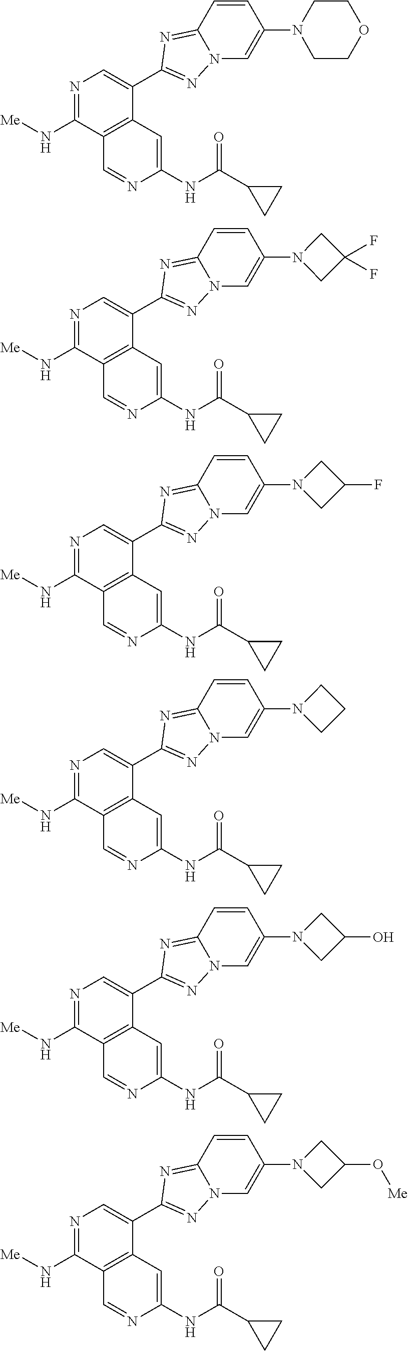

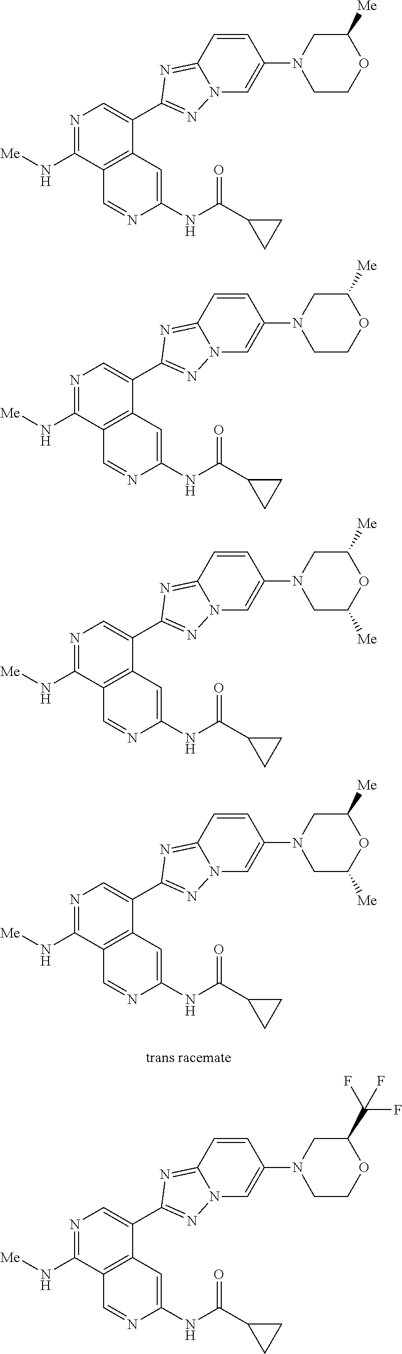 [SMILES: CNc1ncc(-c2nc3ccc(N4CCO[C@H](C)C4)cn3n2)c2cc(NC(=O)C3CC3)ncc12 ; CNc1ncc(-c2nc3ccc(N4CCO[C@@H](C)C4)cn3n2)c2cc(NC(=O)C3CC3)ncc12 ; CNc1ncc(-c2nc3ccc(N4C[C@@H](C)O[C@@H](C)C4)cn3n2)c2cc(NC(=O)C3CC3)ncc12 ; CNc1ncc(-c2nc3ccc(N4C[C@@H](C)O[C@H](C)C4)cn3n2)c2cc(NC(=O)C3CC3)ncc12 ; CNc1ncc(-c2nc3ccc(N4CCO[C@H](C(F)(F)F)C4)cn3n2)c2cc(NC(=O)C3CC3)ncc12]

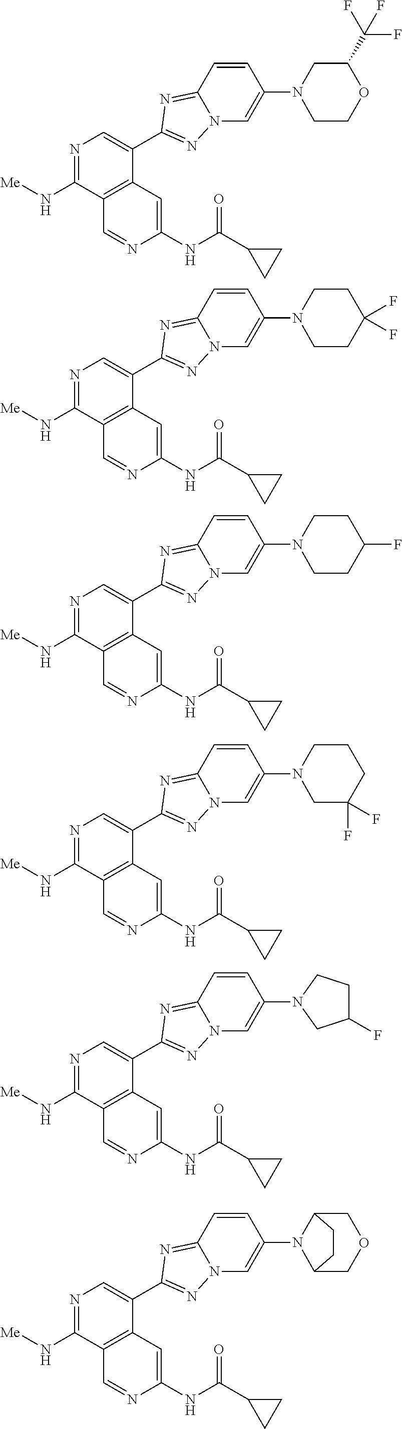

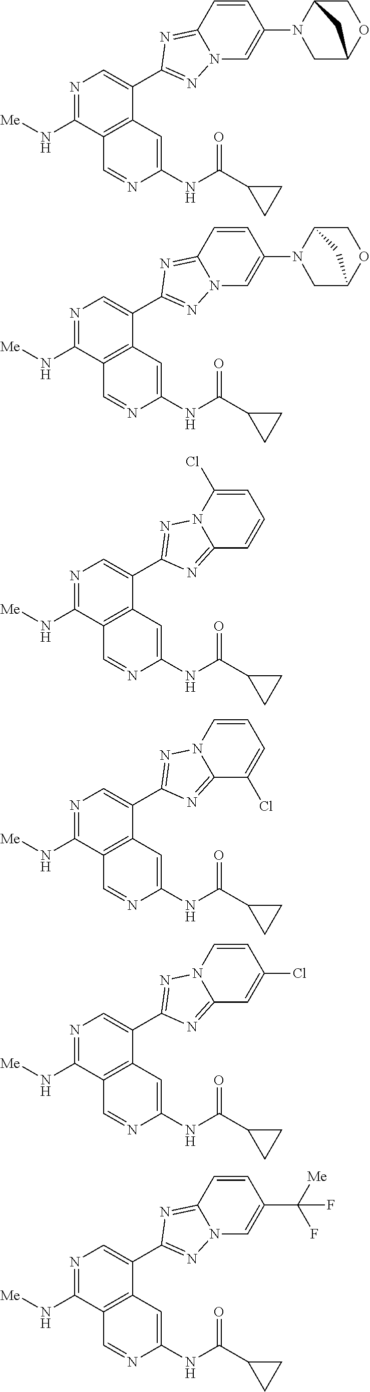

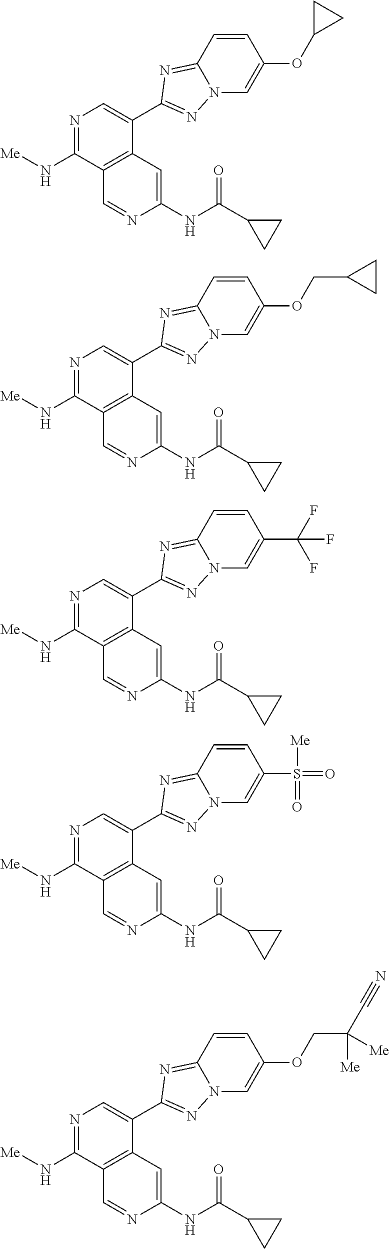

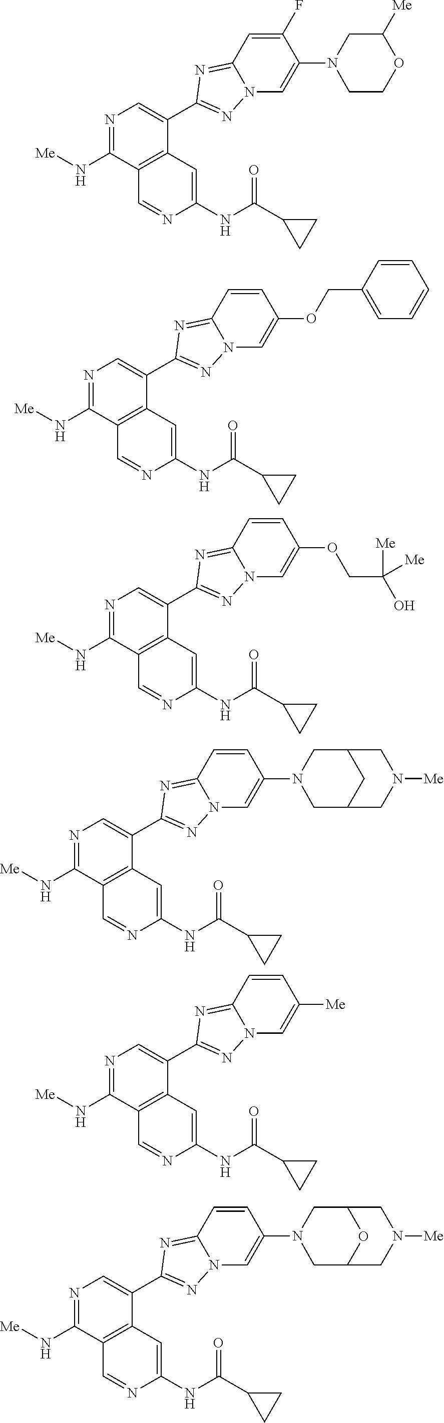

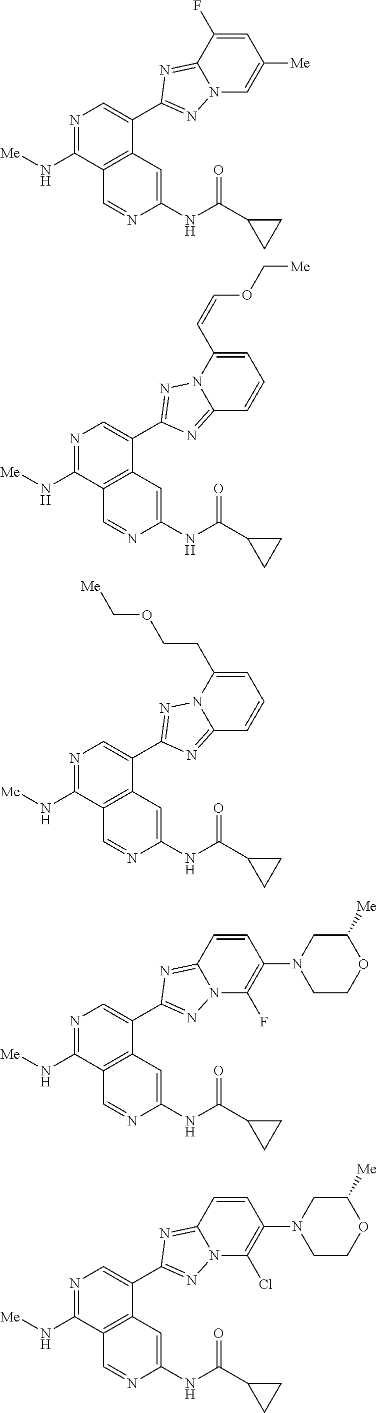

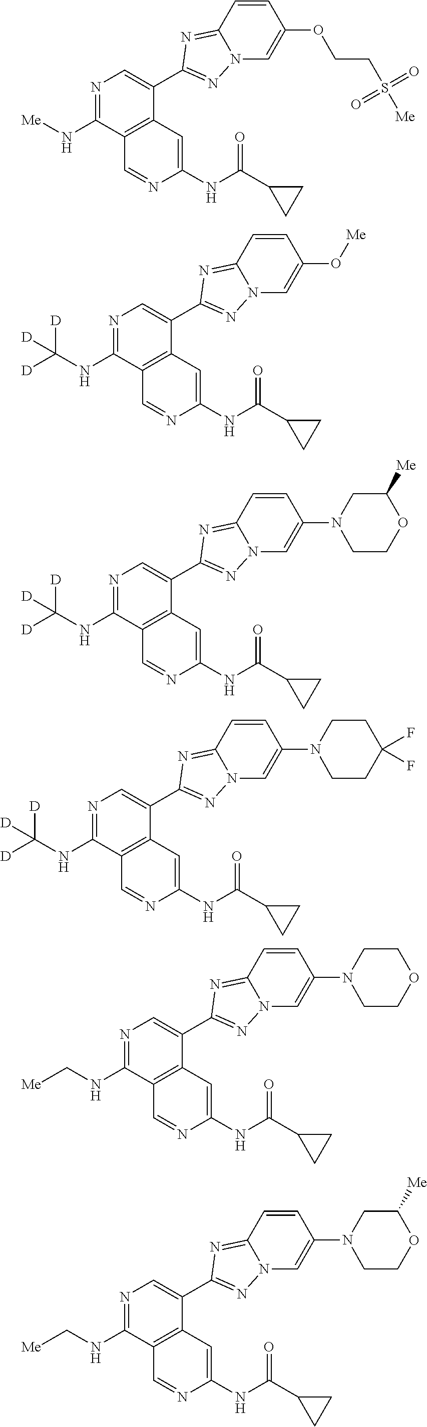

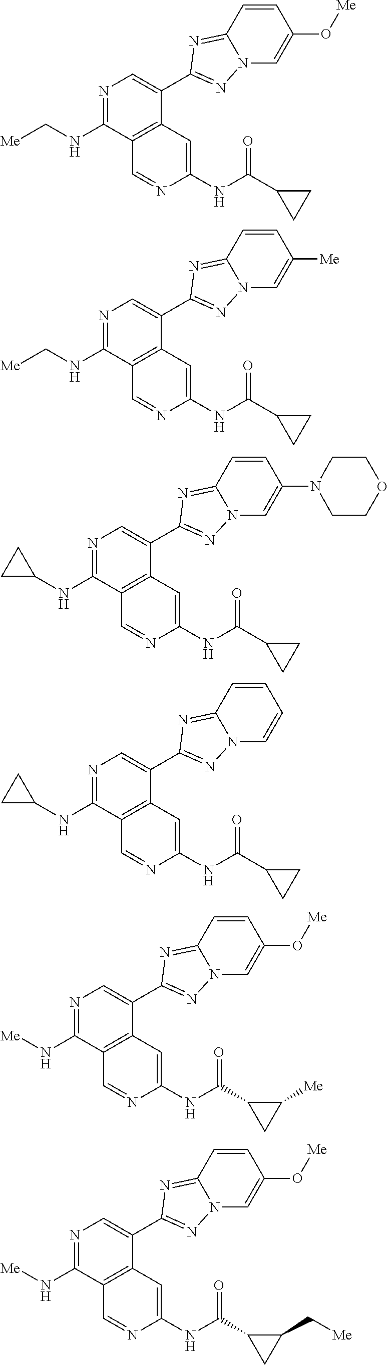

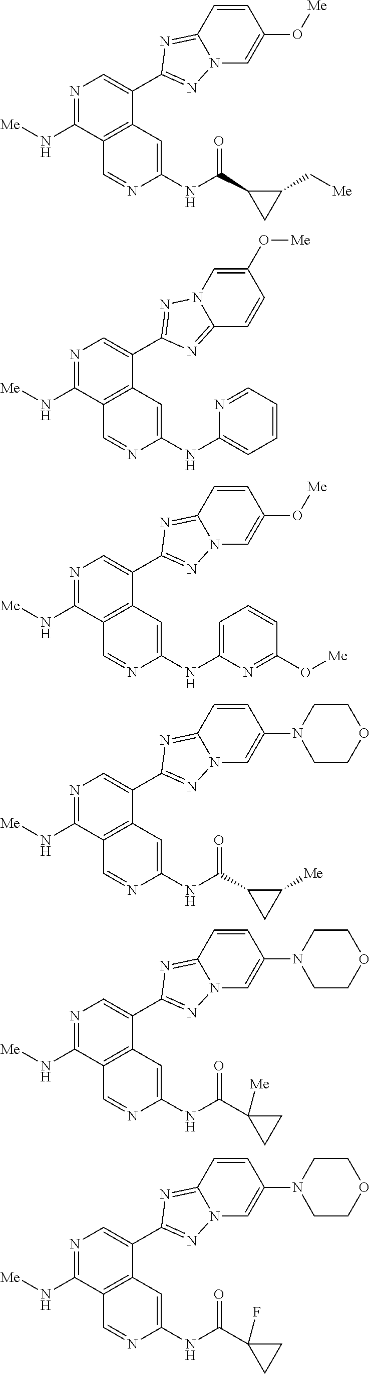

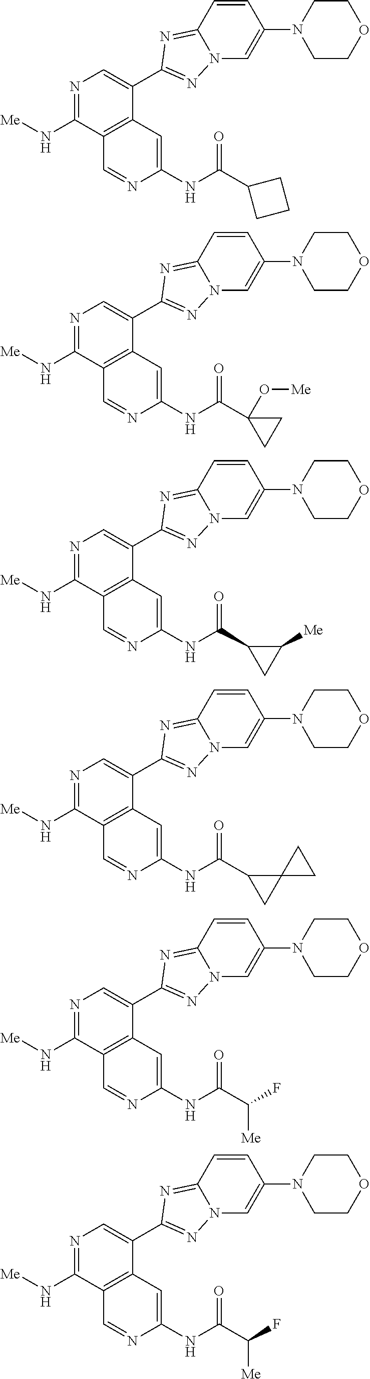

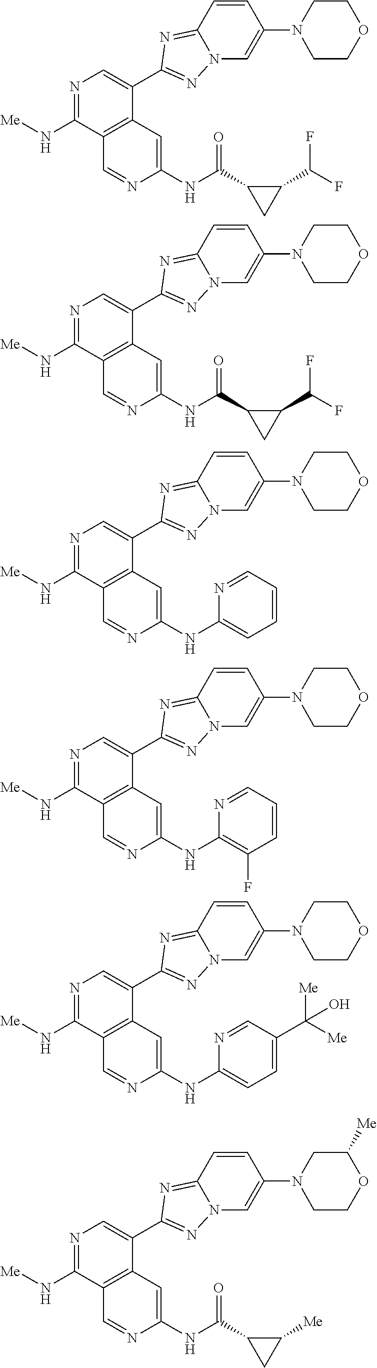

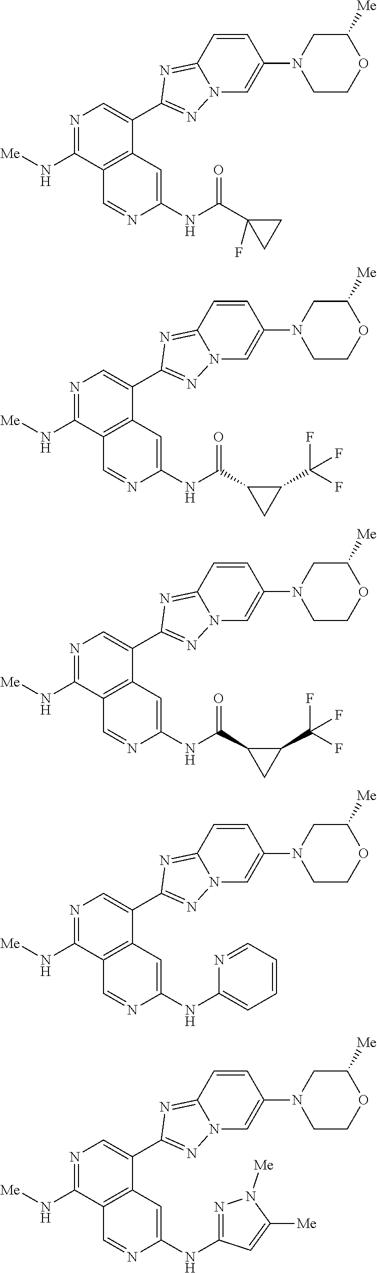 [SMILES: CNc1ncc(-c2nc3ccc(N4CCO[C@@H](C)C4)cn3n2)c2cc(NC(=O)C3(F)CC3)ncc12 ; CNc1ncc(-c2nc3ccc(N4CCO[C@@H](C)C4)cn3n2)c2cc(NC(=O)[C@H]3C[C@H]3C(F)(F)F)ncc12 ; CNc1ncc(-c2nc3ccc(N4CCO[C@@H](C)C4)cn3n2)c2cc(NC(=O)[C@@H]3C[C@@H]3C(F)(F)F)ncc12 ; CNc1ncc(-c2nc3ccc(N4CCO[C@@H](C)C4)cn3n2)c2cc(Nc3ccccn3)ncc12 ; CNc1ncc(-c2nc3ccc(N4CCO[C@@H](C)C4)cn3n2)c2cc(Nc3cc(C)n(C)n3)ncc12]

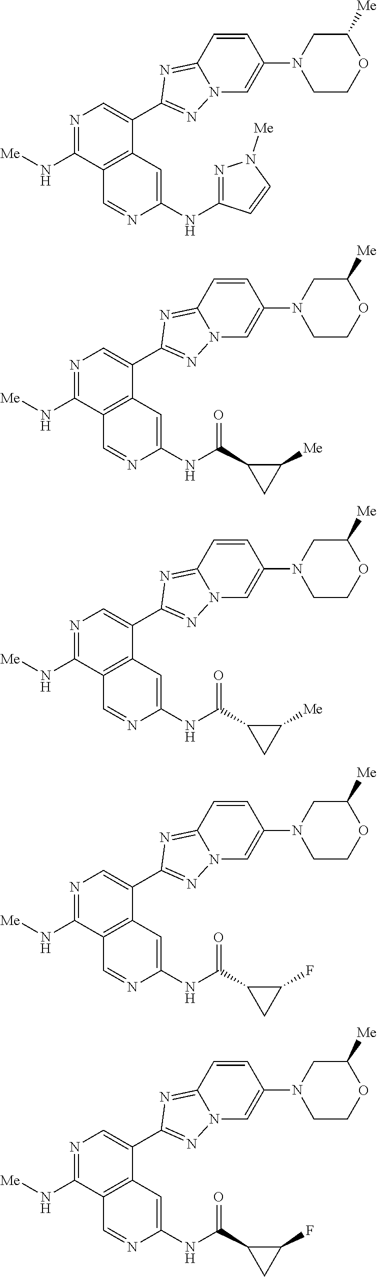 [SMILES: CNc1ncc(-c2nc3ccc(N4CCO[C@@H](C)C4)cn3n2)c2cc(Nc3ccn(C)n3)ncc12 ; CNc1ncc(-c2nc3ccc(N4CCO[C@H](C)C4)cn3n2)c2cc(NC(=O)[C@@H]3C[C@@H]3C)ncc12 ; CNc1ncc(-c2nc3ccc(N4CCO[C@H](C)C4)cn3n2)c2cc(NC(=O)[C@H]3C[C@H]3C)ncc12 ; CNc1ncc(-c2nc3ccc(N4CCO[C@H](C)C4)cn3n2)c2cc(NC(=O)[C@H]3C[C@H]3F)ncc12 ; CNc1ncc(-c2nc3ccc(N4CCO[C@H](C)C4)cn3n2)c2cc(NC(=O)[C@@H]3C[C@@H]3F)ncc12]

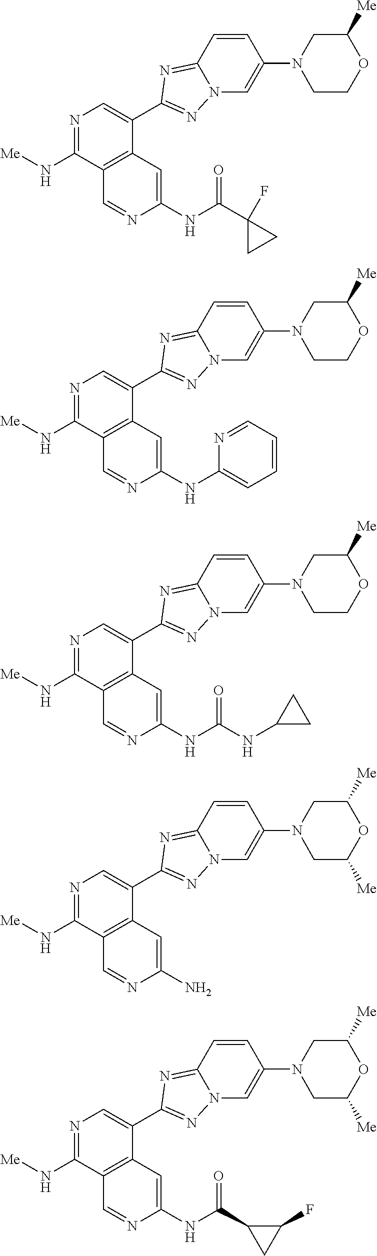 [SMILES: CNc1ncc(-c2nc3ccc(N4CCO[C@H](C)C4)cn3n2)c2cc(NC(=O)C3(F)CC3)ncc12 ; CNc1ncc(-c2nc3ccc(N4CCO[C@H](C)C4)cn3n2)c2cc(Nc3ccccn3)ncc12 ; CNc1ncc(-c2nc3ccc(N4CCO[C@H](C)C4)cn3n2)c2cc(NC(=O)NC3CC3)ncc12 ; CNc1ncc(-c2nc3ccc(N4C[C@@H](C)O[C@@H](C)C4)cn3n2)c2cc(N)ncc12 ; CNc1ncc(-c2nc3ccc(N4C[C@@H](C)O[C@@H](C)C4)cn3n2)c2cc(NC(=O)[C@@H]3C[C@@H]3F)ncc12]

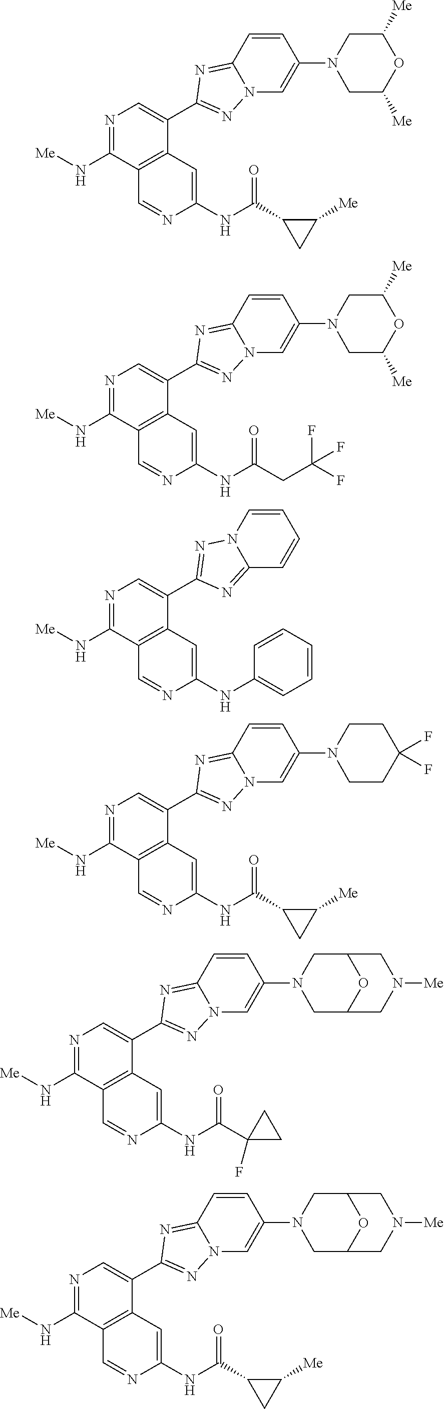

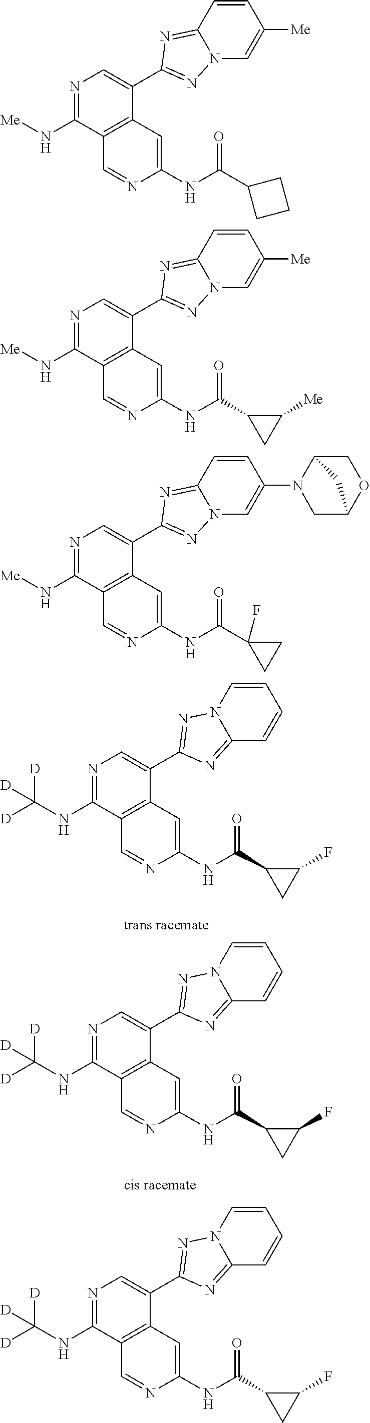

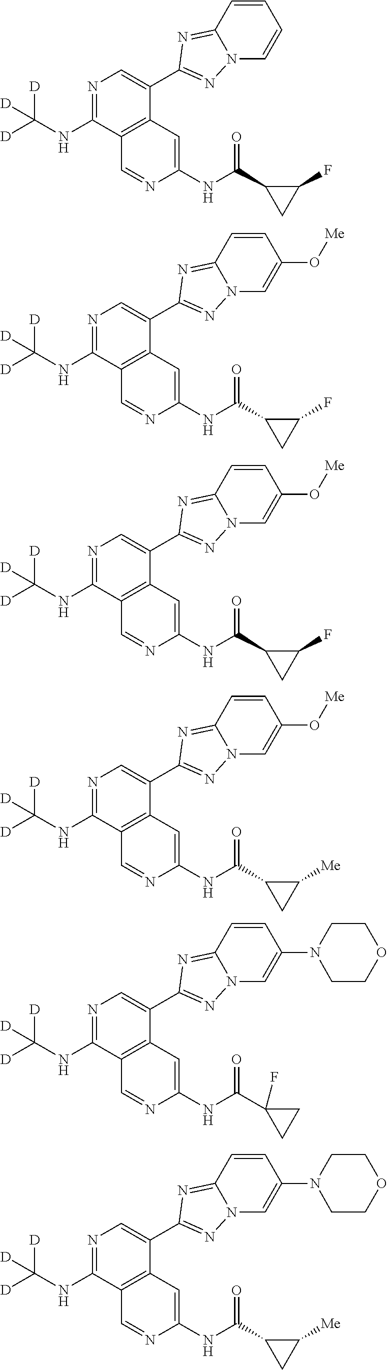

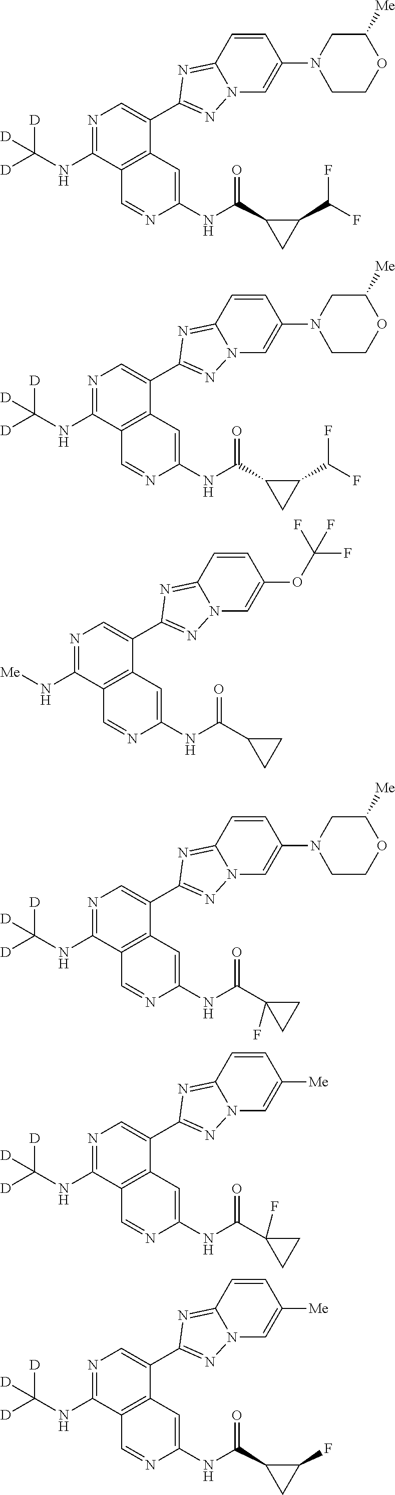

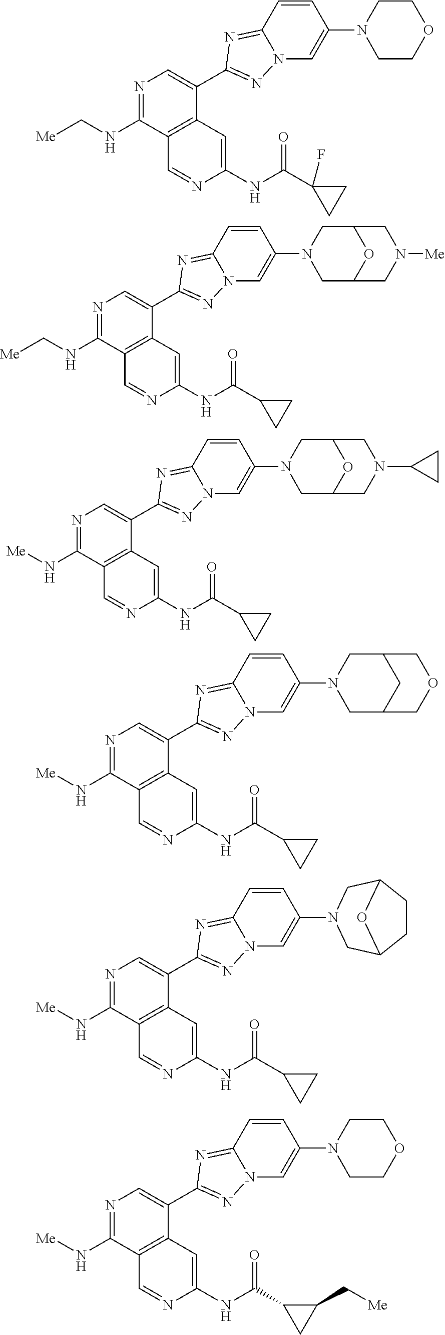

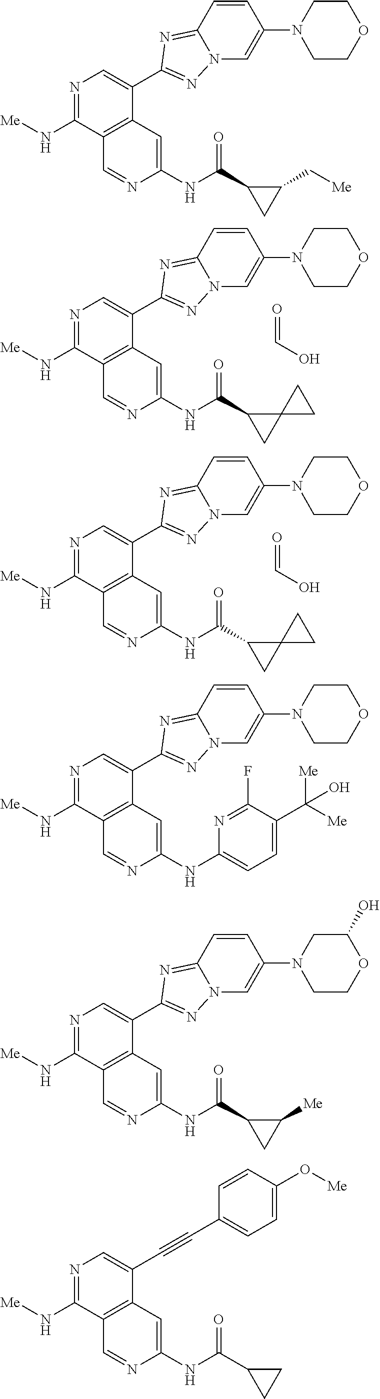

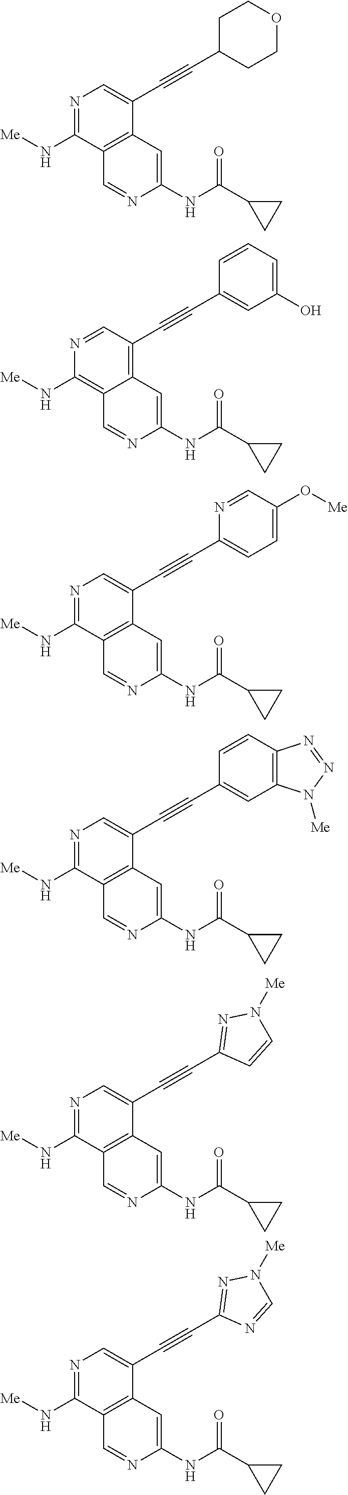

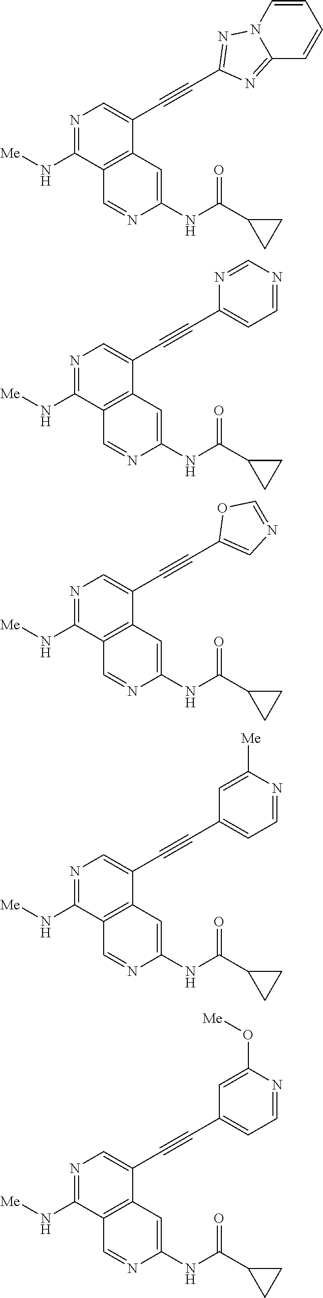

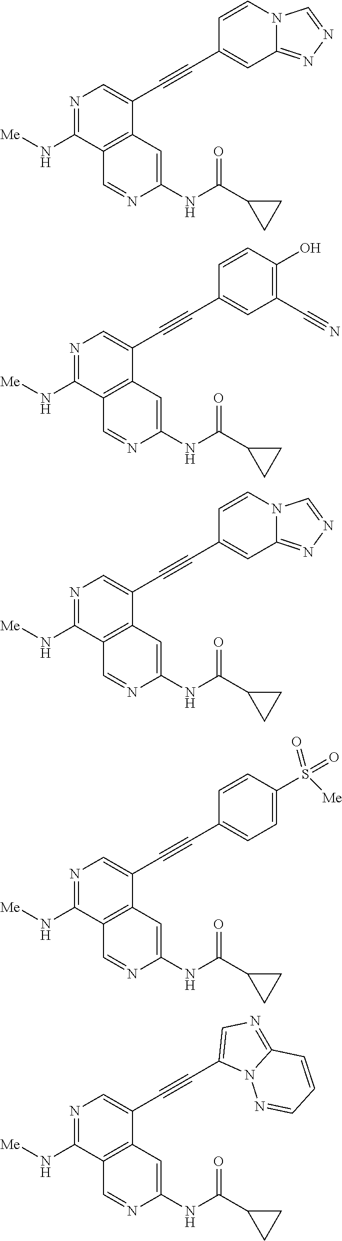

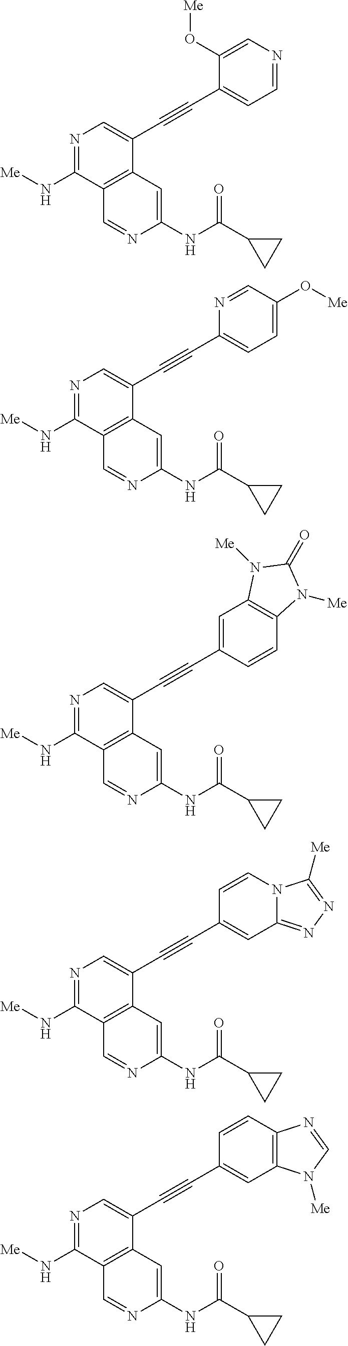

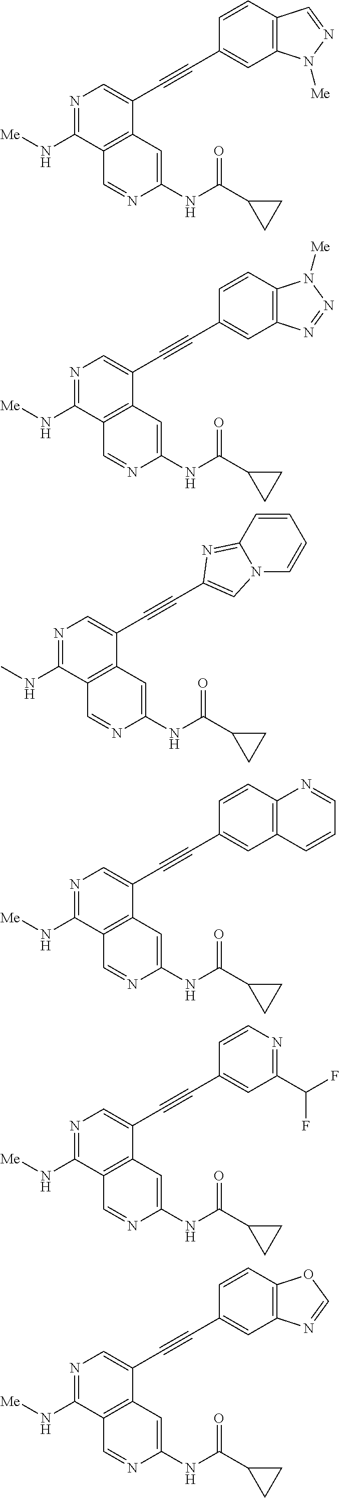

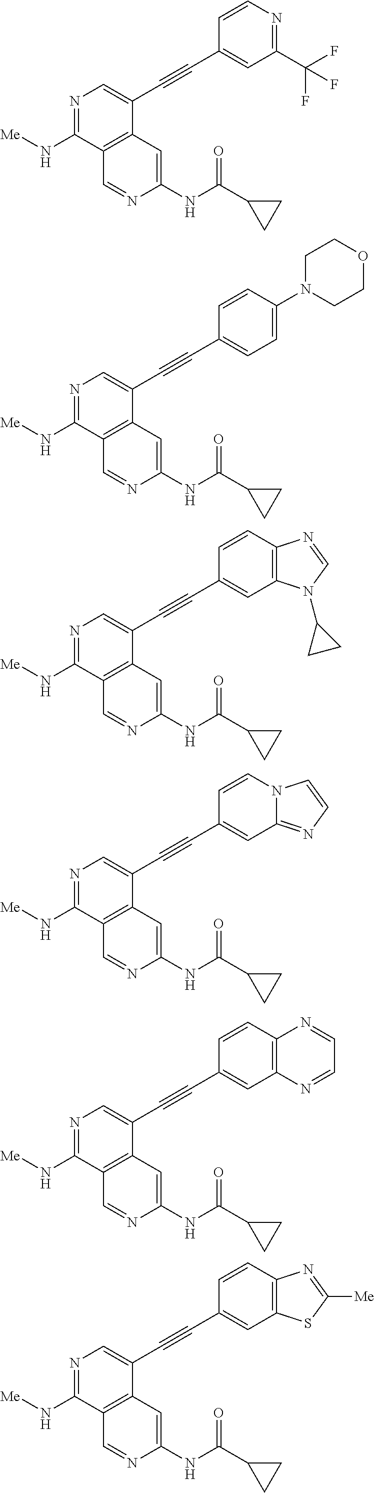

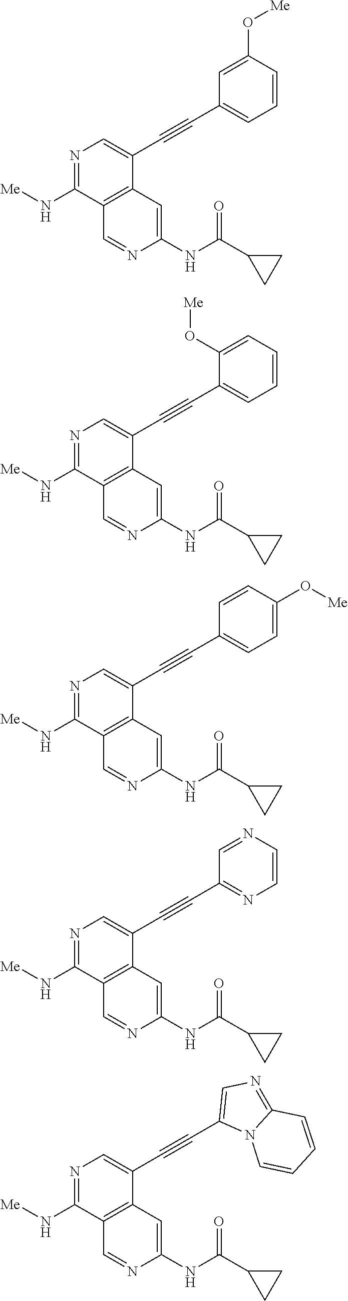

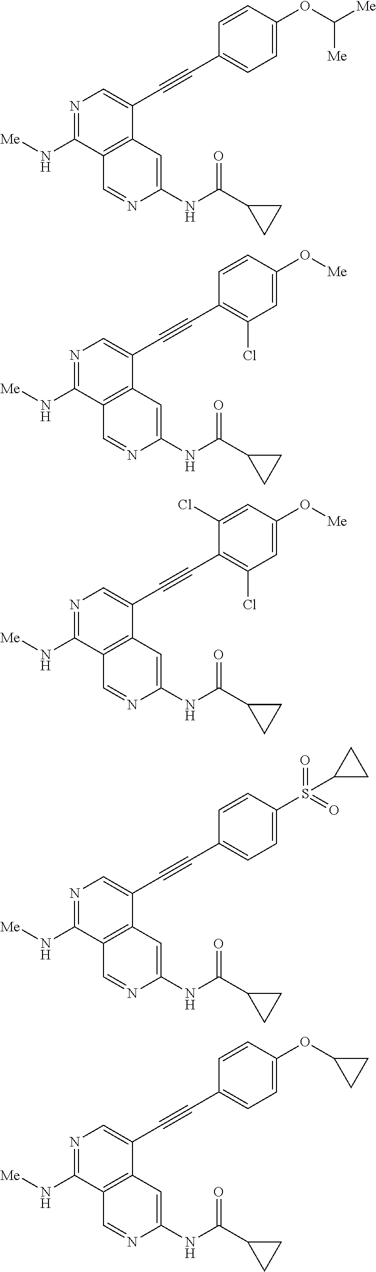

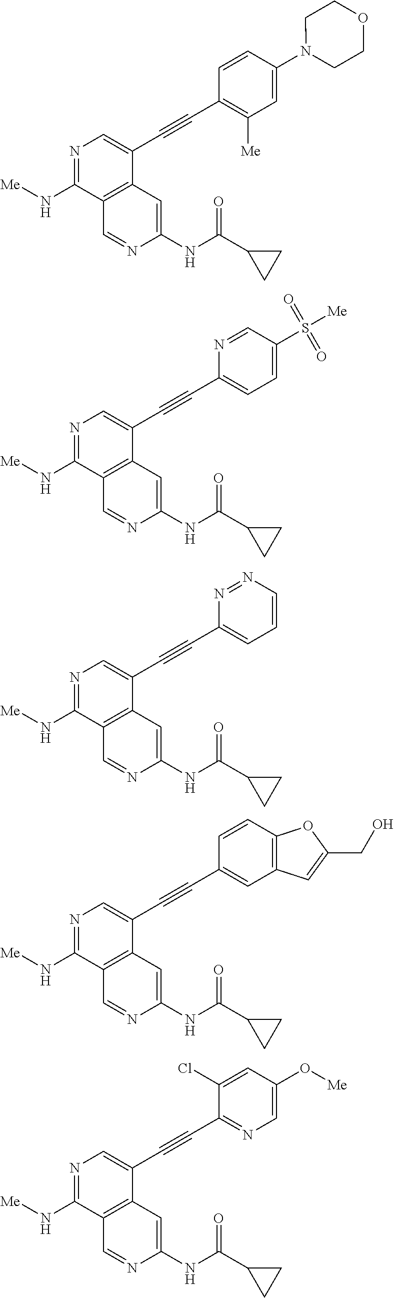

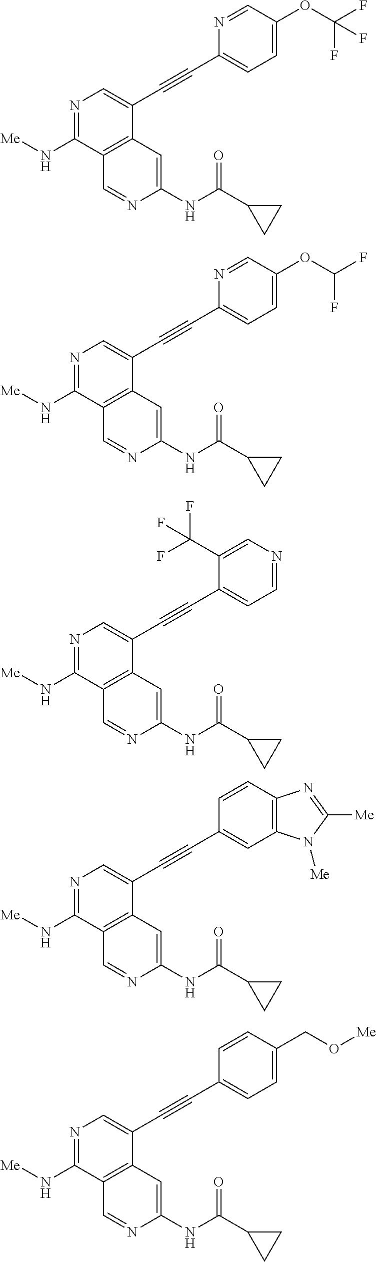

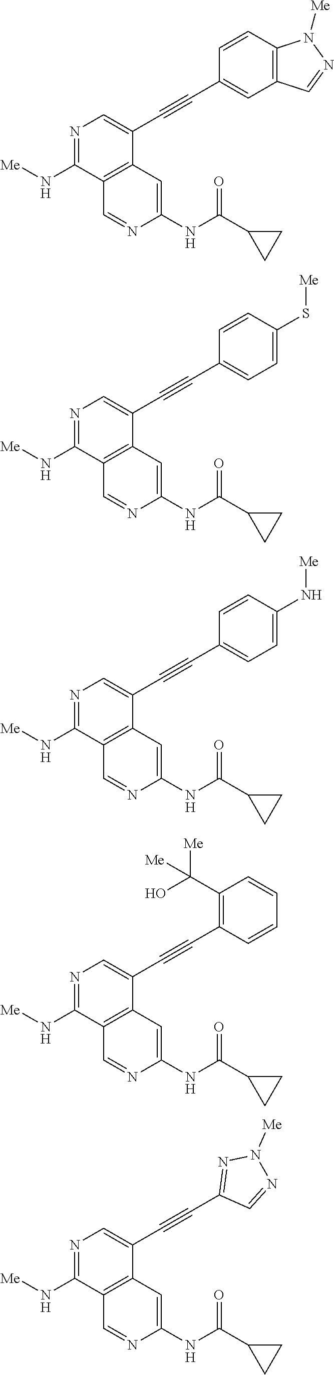

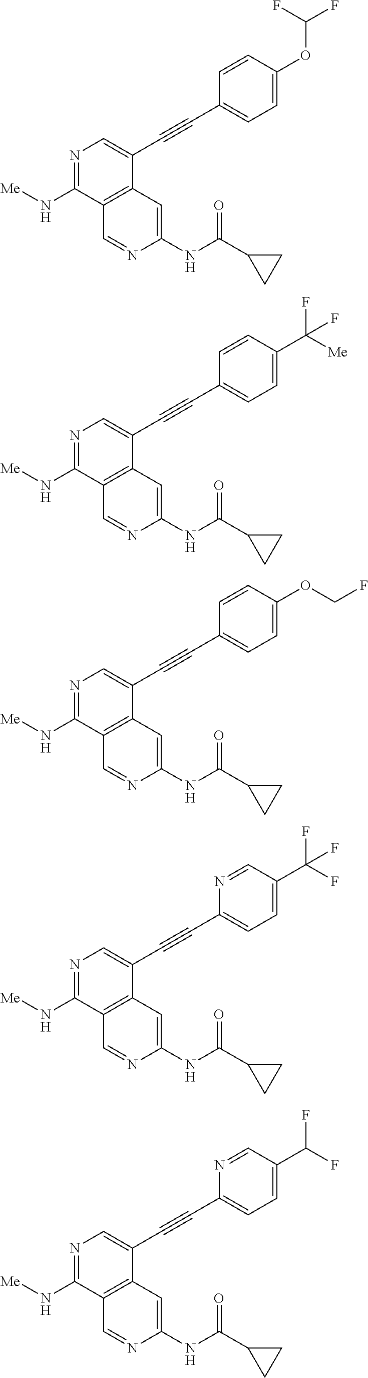

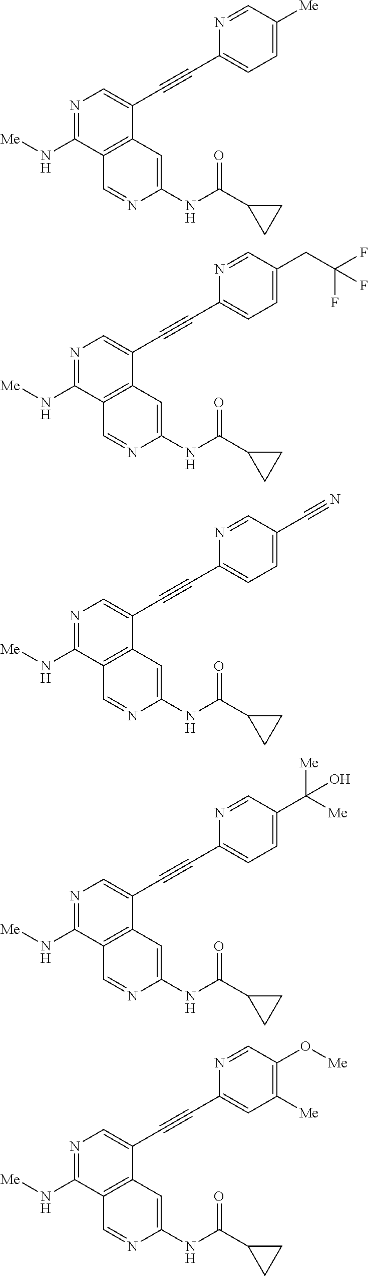

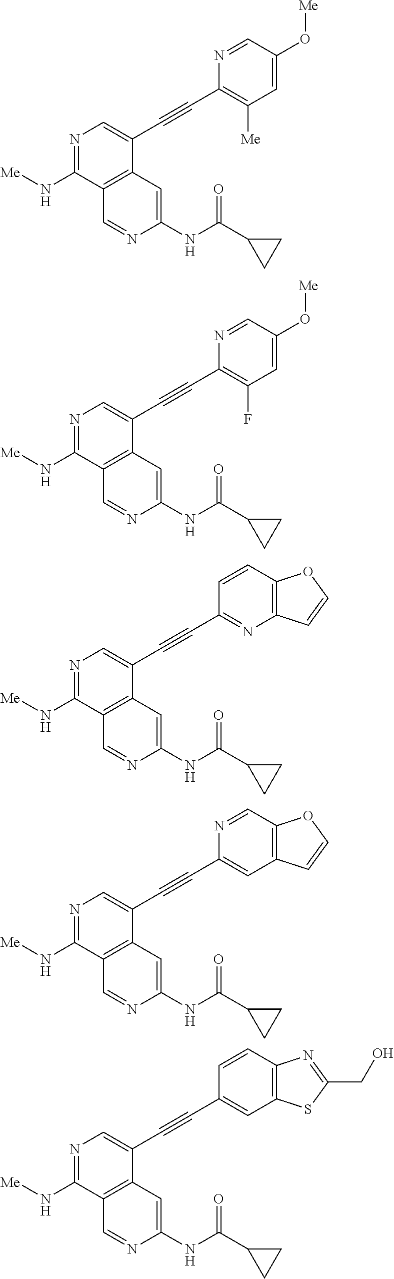

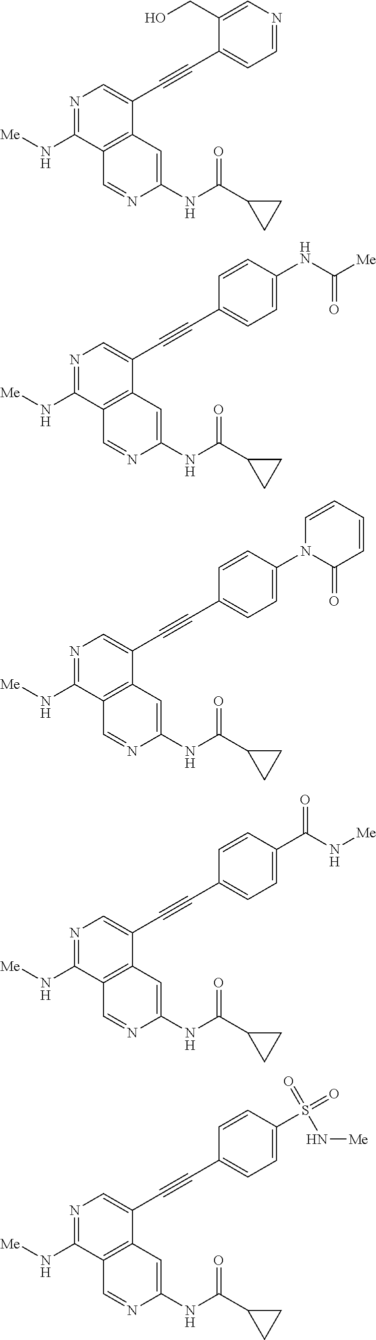

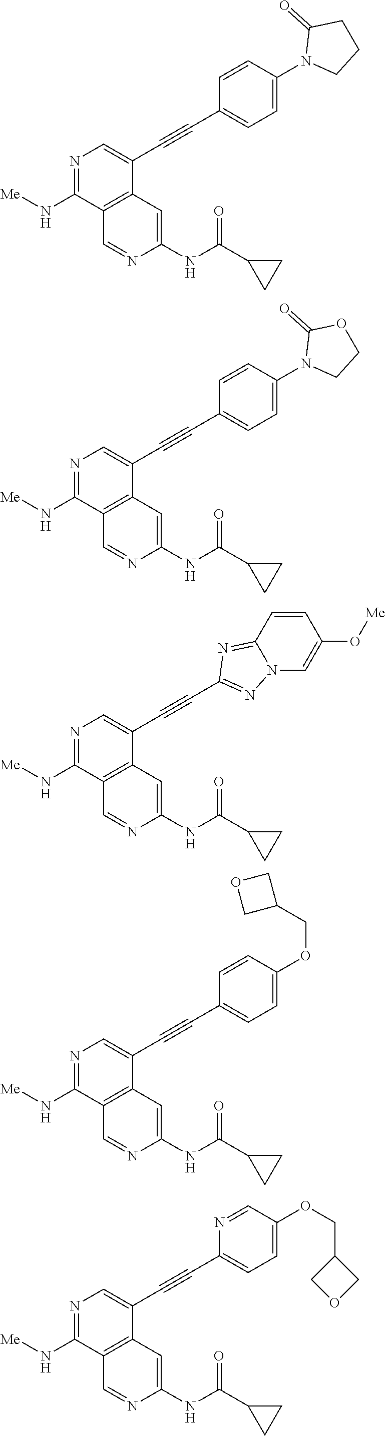

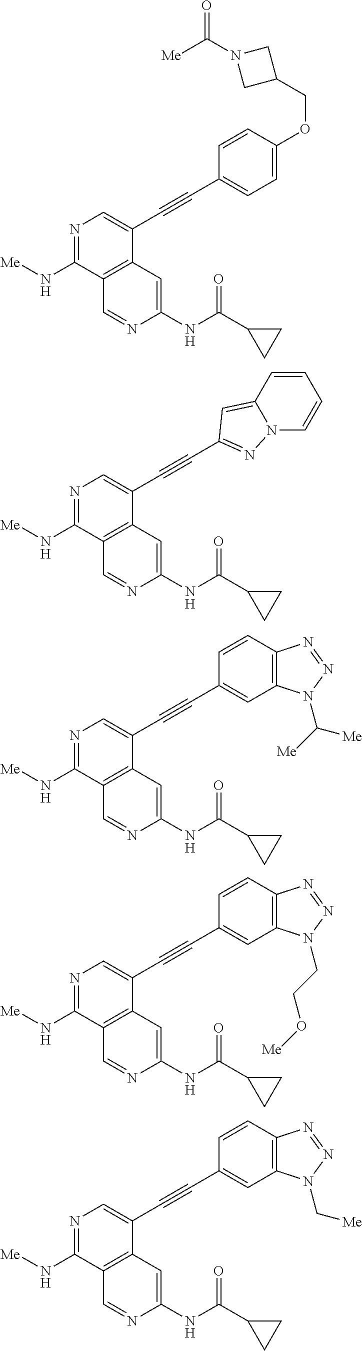

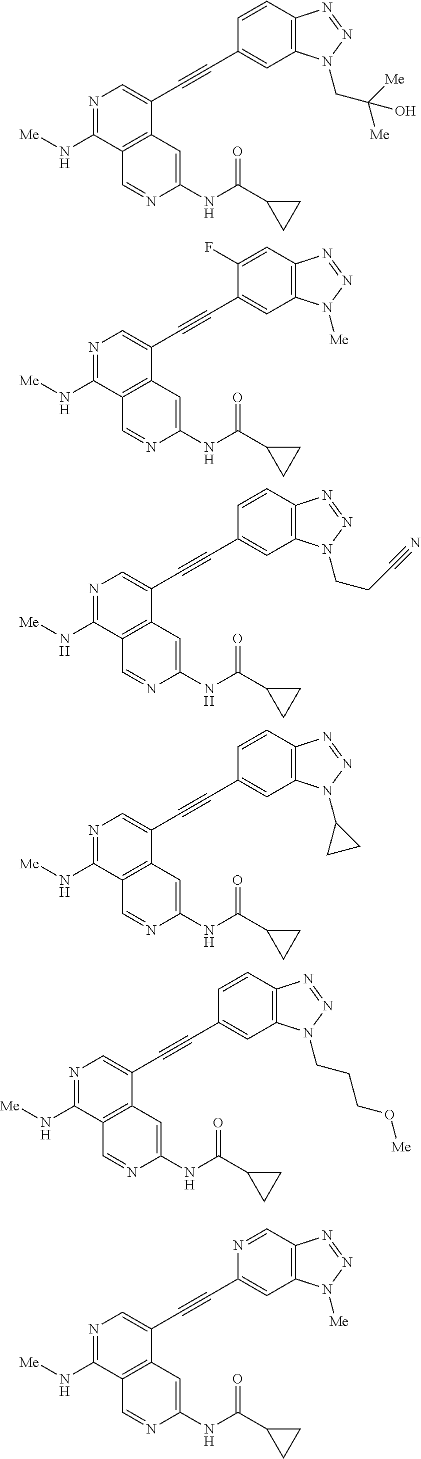

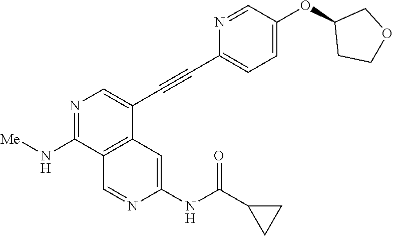 [SMILES: CNc1ncc(C#Cc2ccc(O[C@@H]3CCOC3)cn2)c2cc(NC(=O)C3CC3)ncc12]

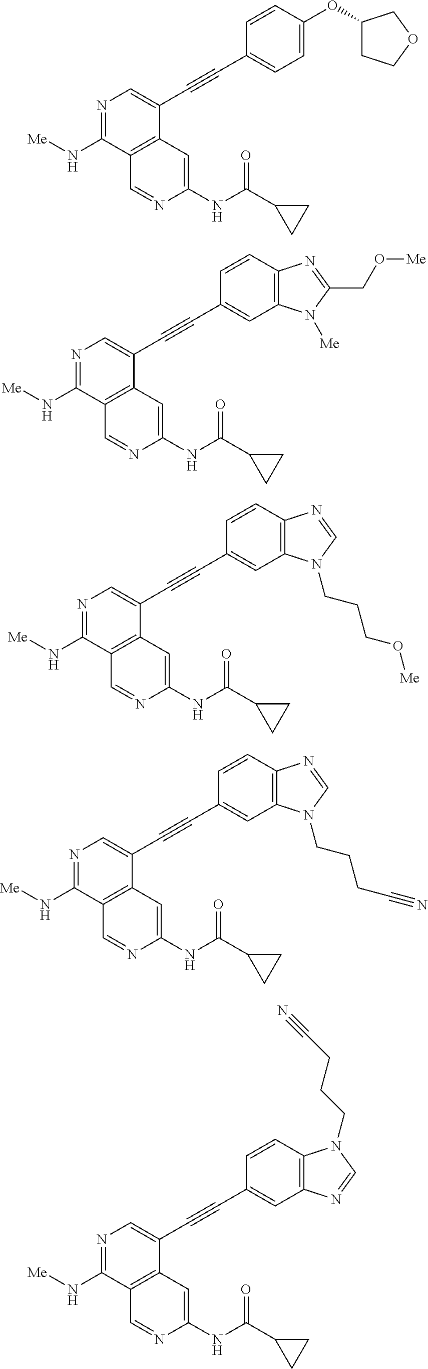

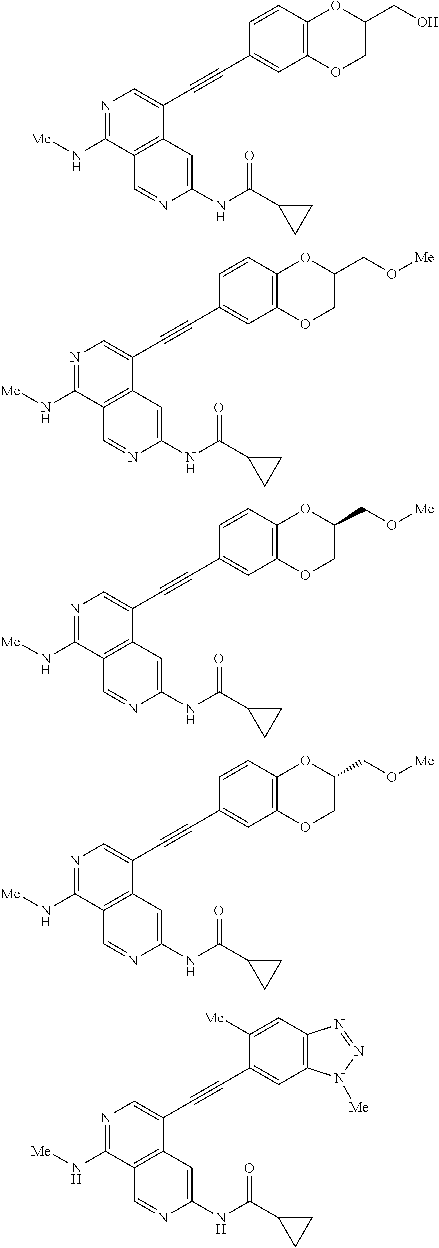

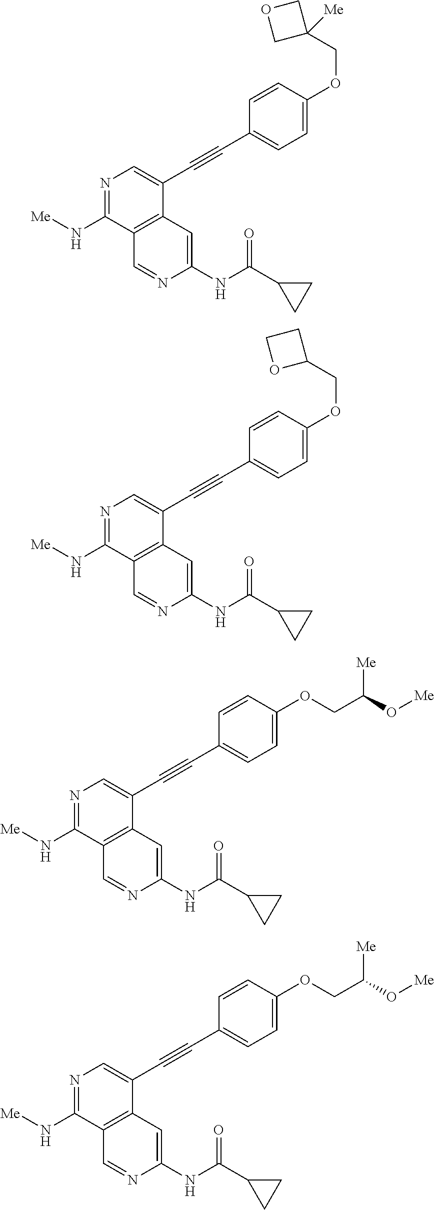

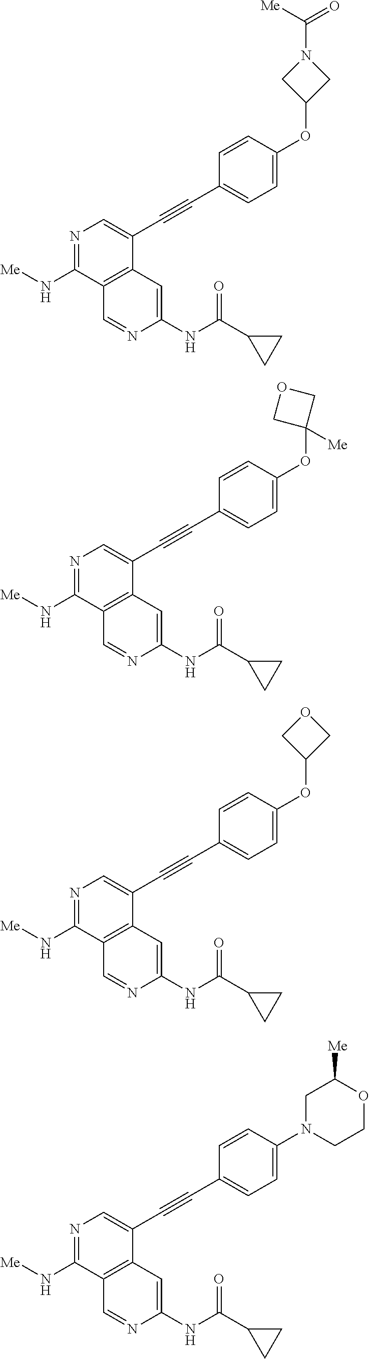

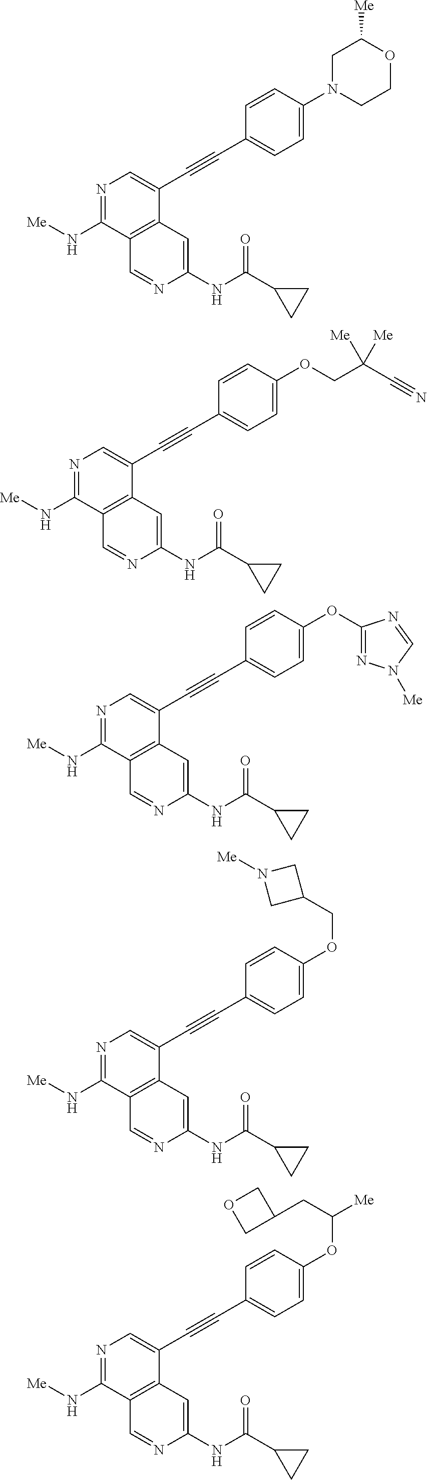

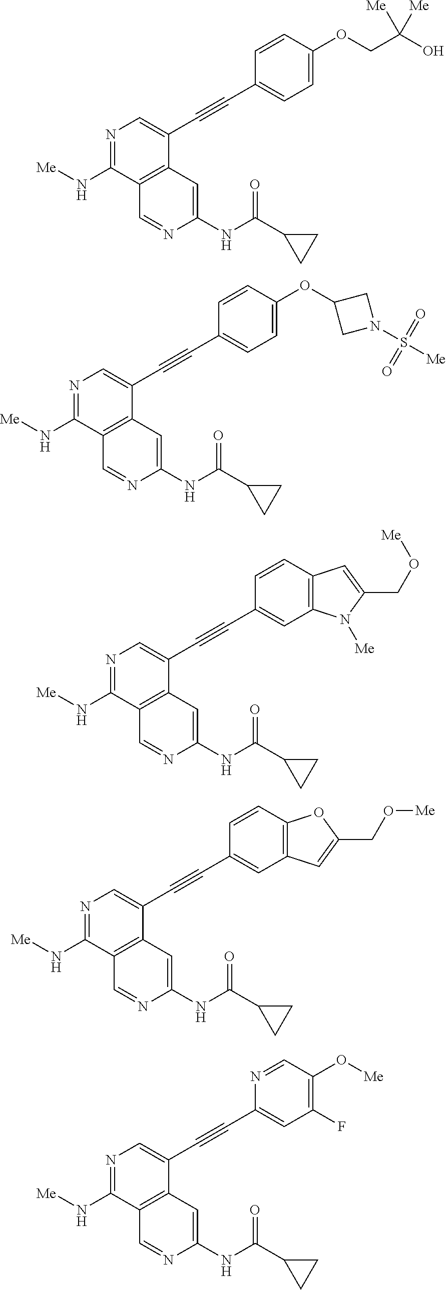

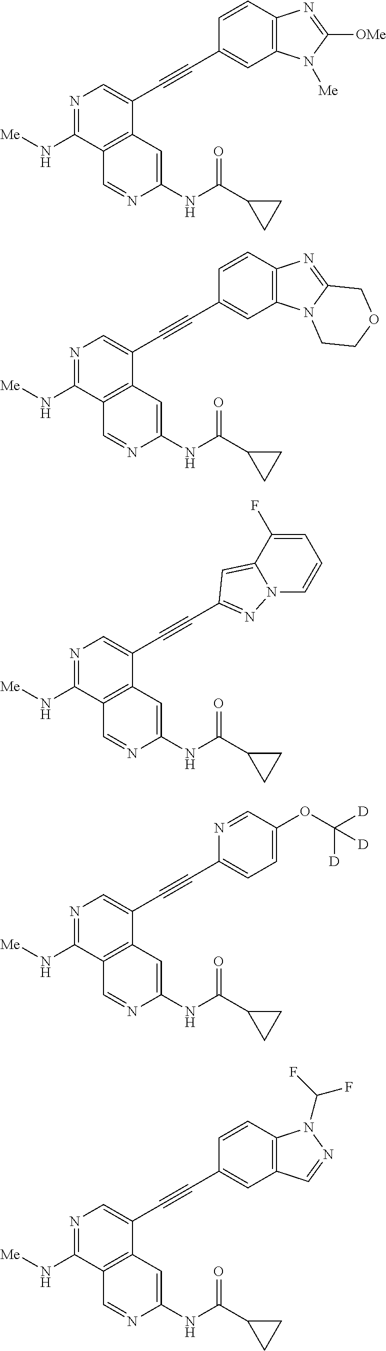

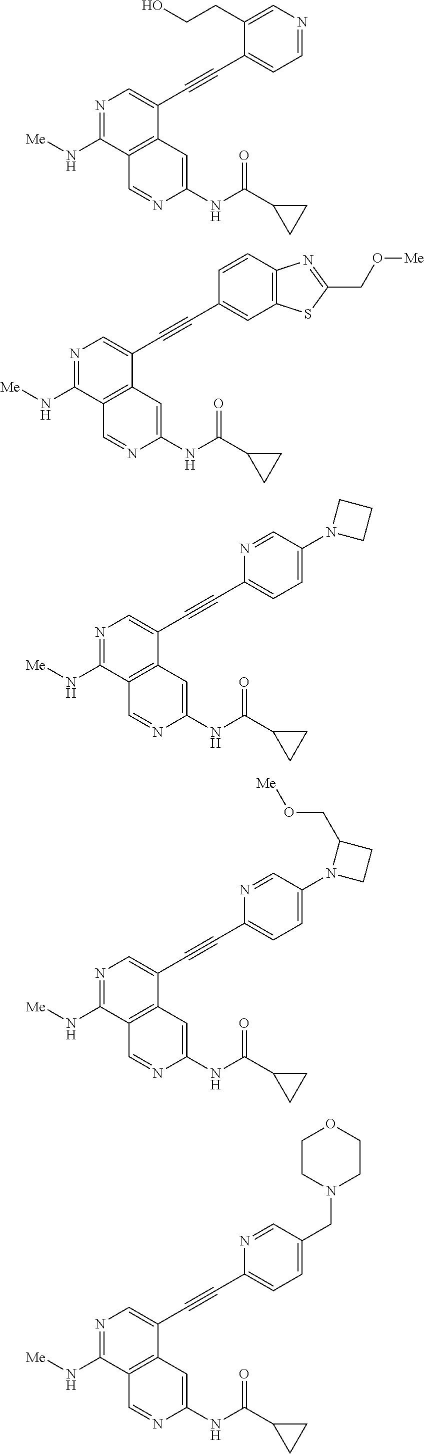

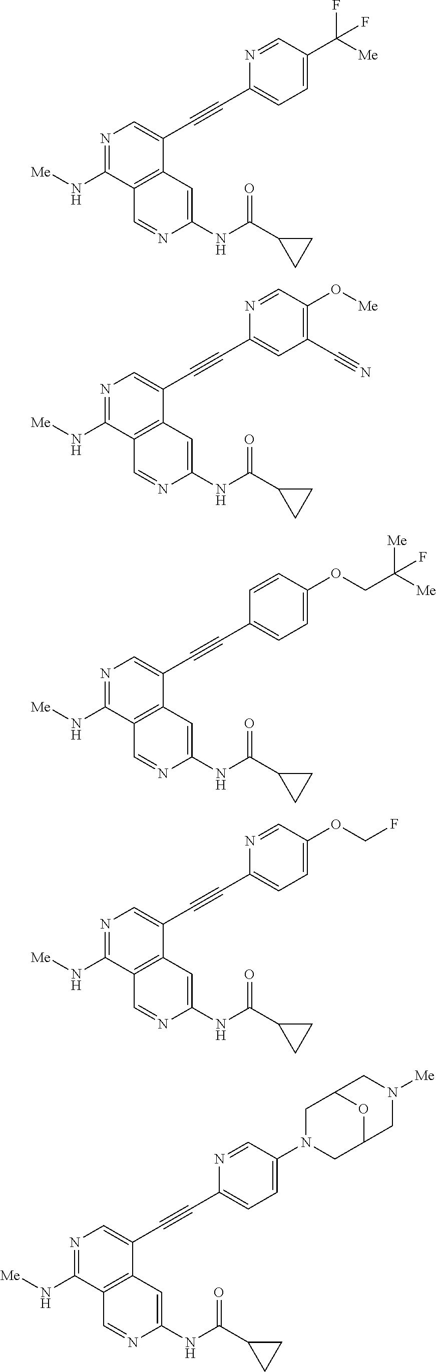

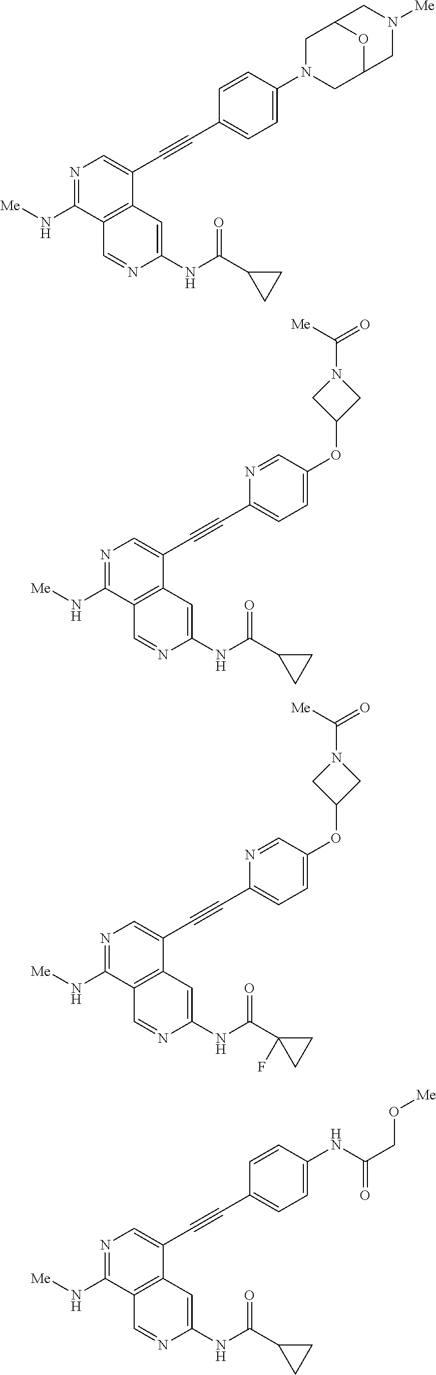

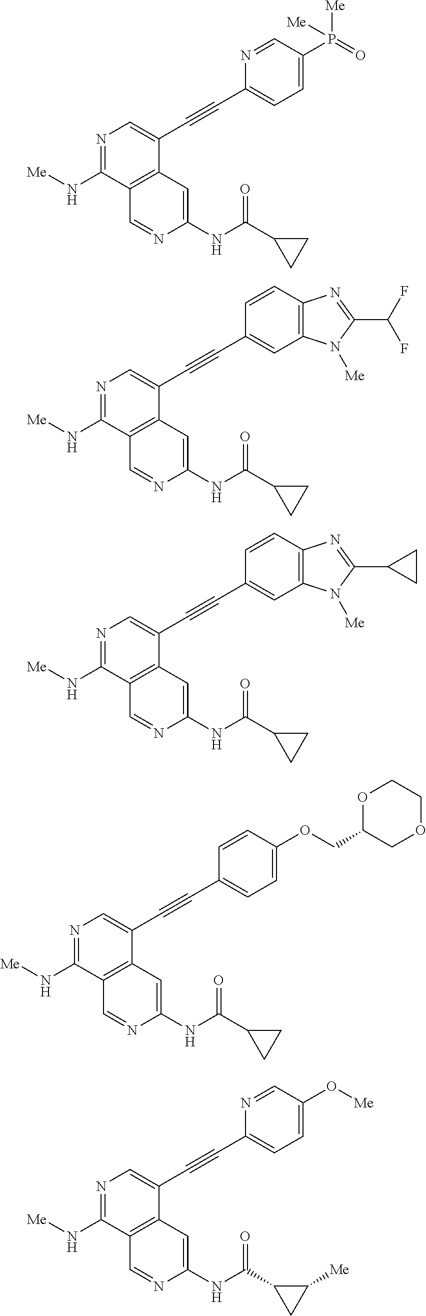

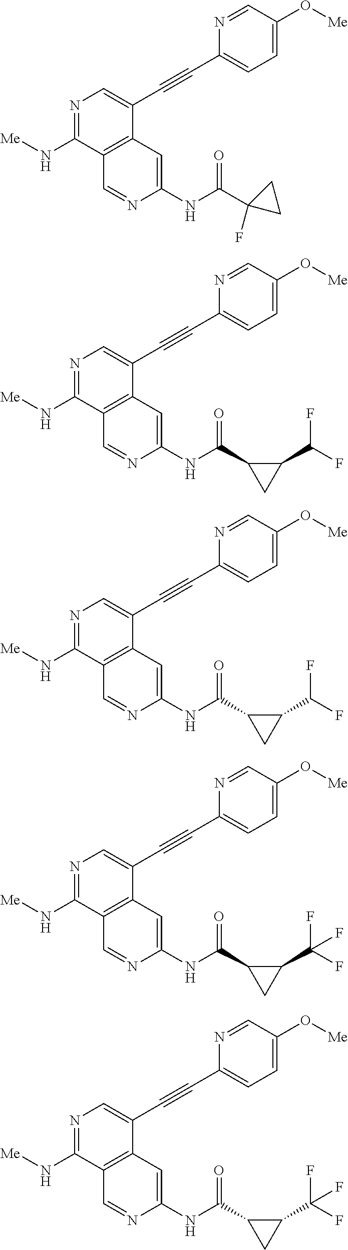

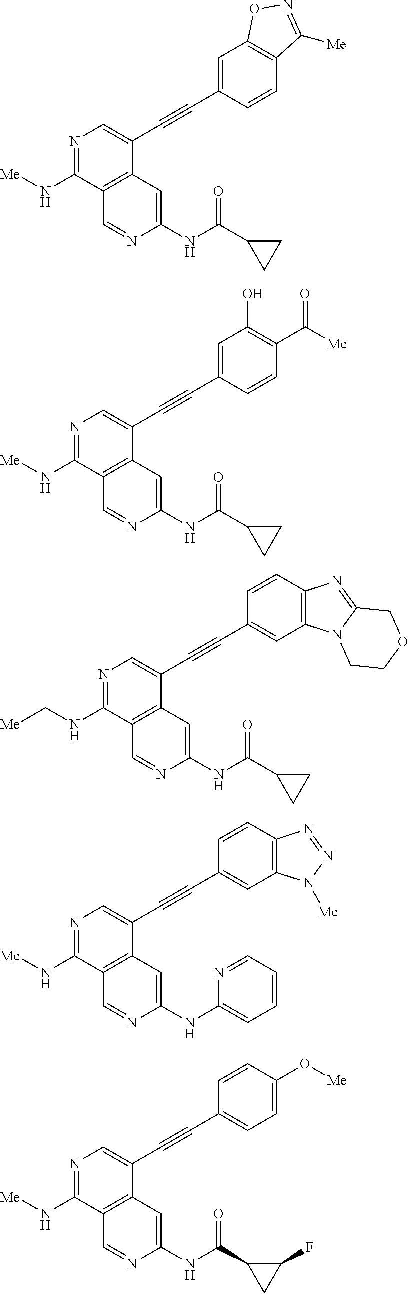

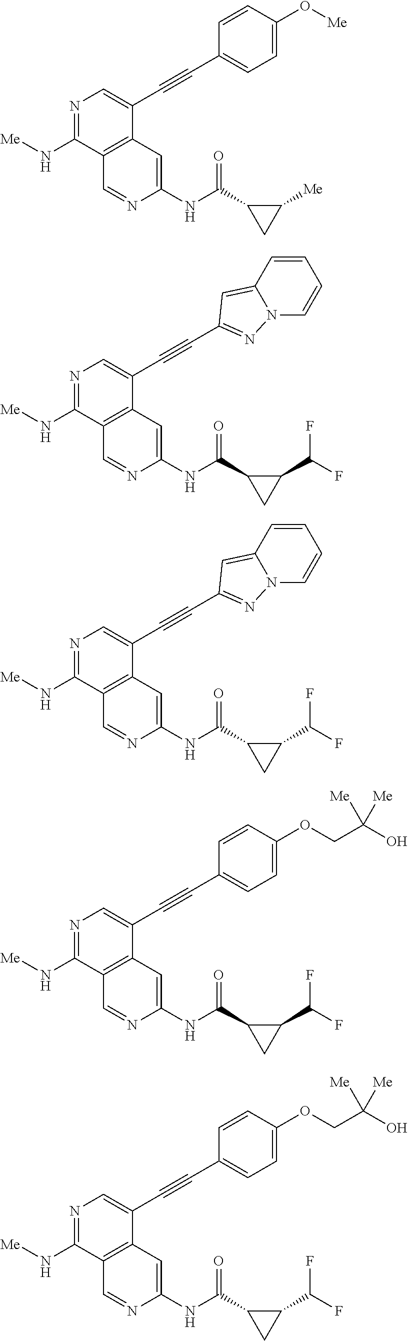

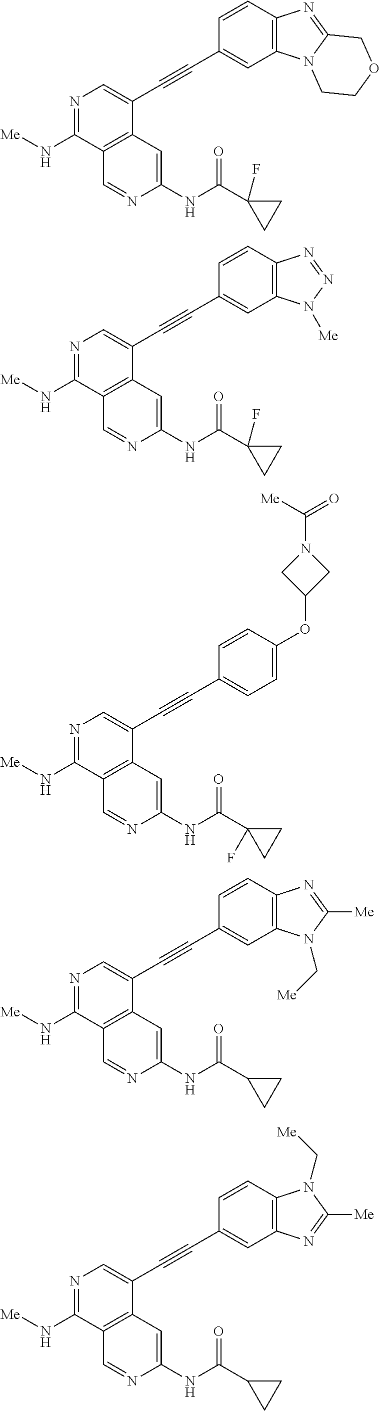

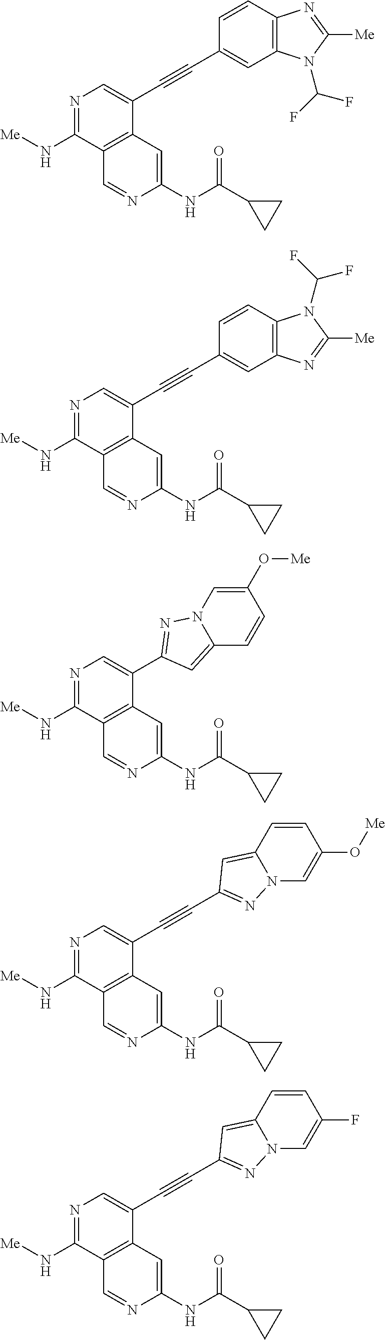

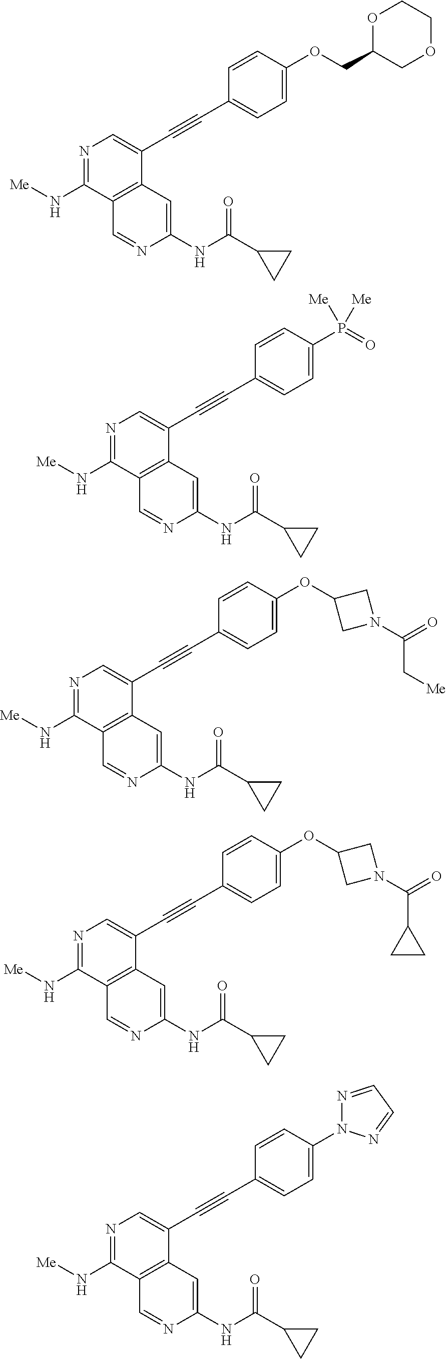

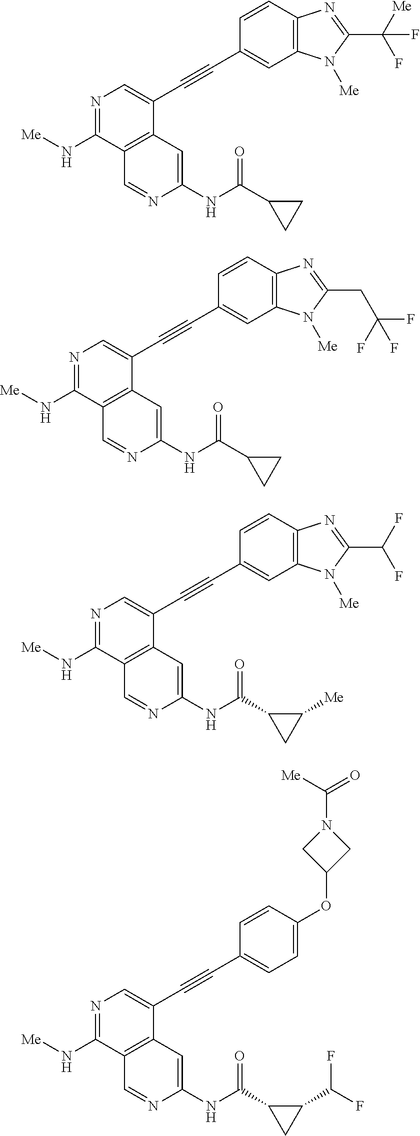

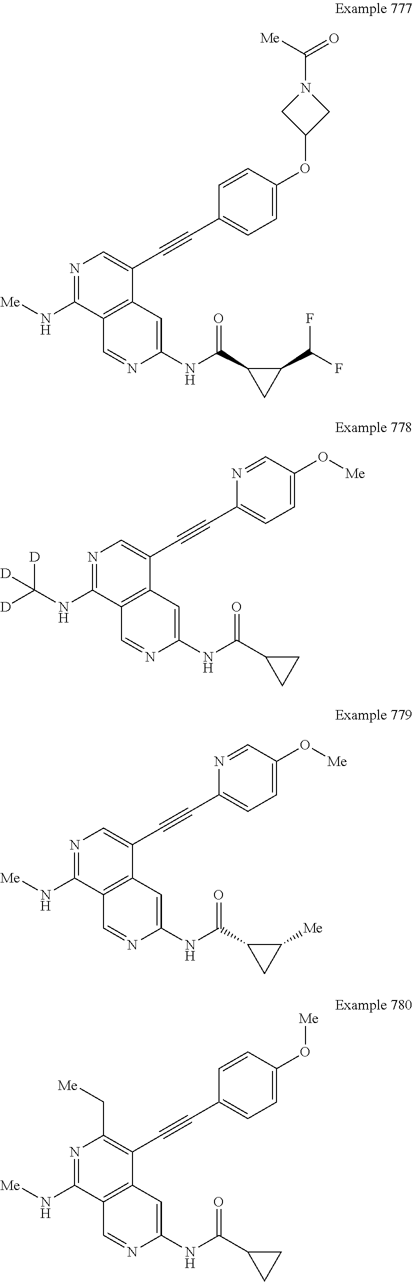 [SMILES: CNc1ncc(C#Cc2ccc(OC3CN(C(C)=O)C3)cc2)c2cc(NC(=O)[C@@H]3C[C@@H]3C(F)F)ncc12 ; [2H]C([2H])([2H])Nc1ncc(C#Cc2ccc(OC)cn2)c2cc(NC(=O)C3CC3)ncc12 ; CNc1ncc(C#Cc2ccc(OC)cn2)c2cc(NC(=O)[C@H]3C[C@H]3C)ncc12 ; CCc1nc(NC)c2cnc(NC(=O)C3CC3)cc2c1C#Cc1ccc(OC)cc1]

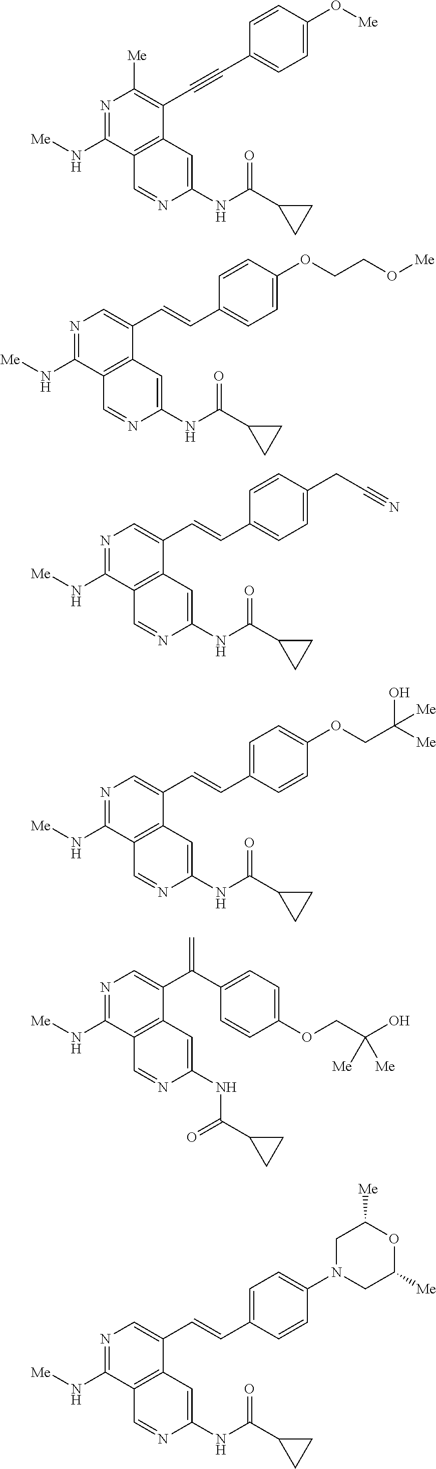

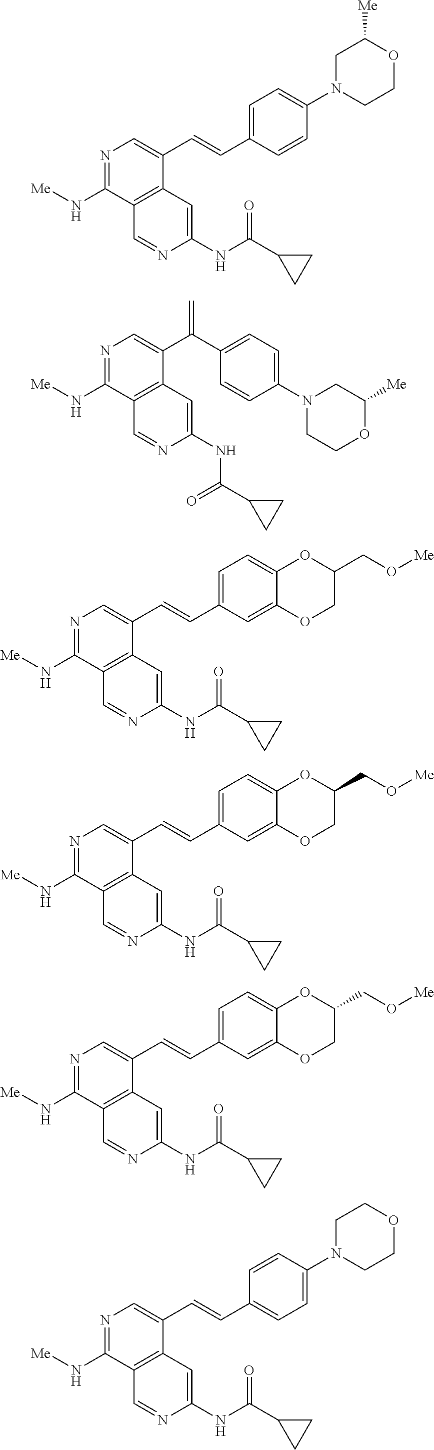

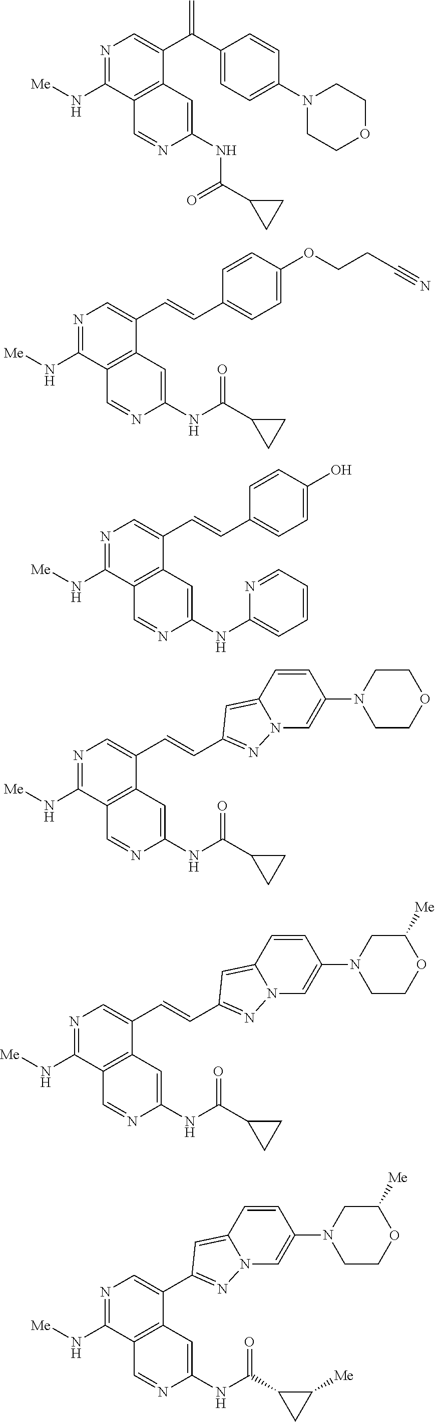

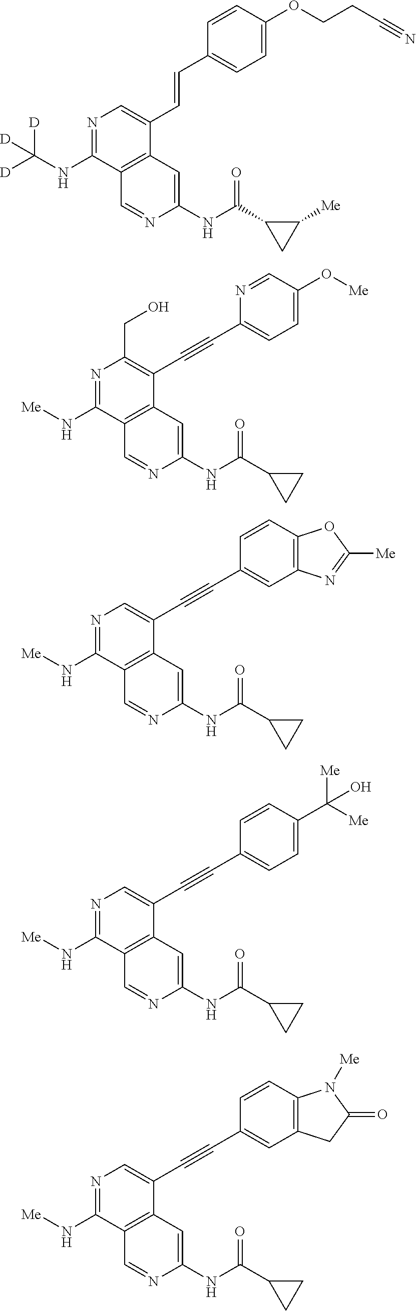

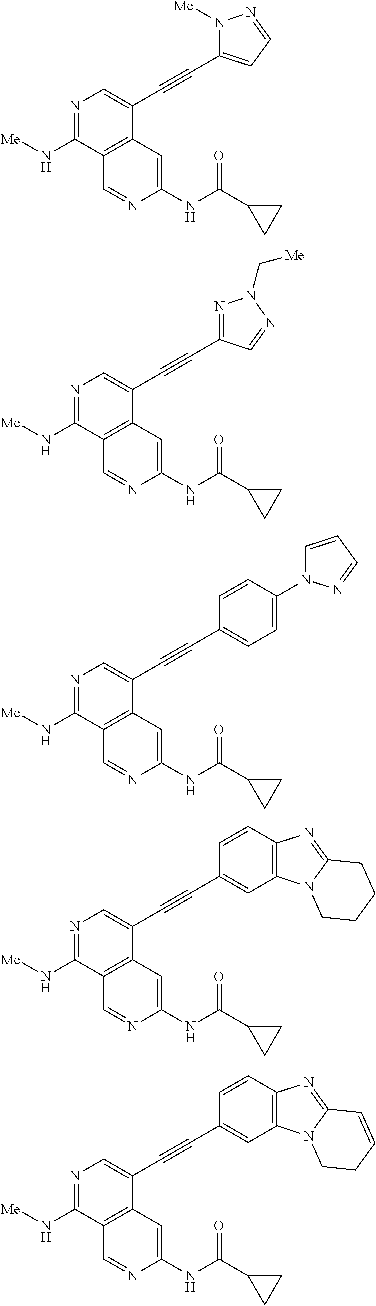

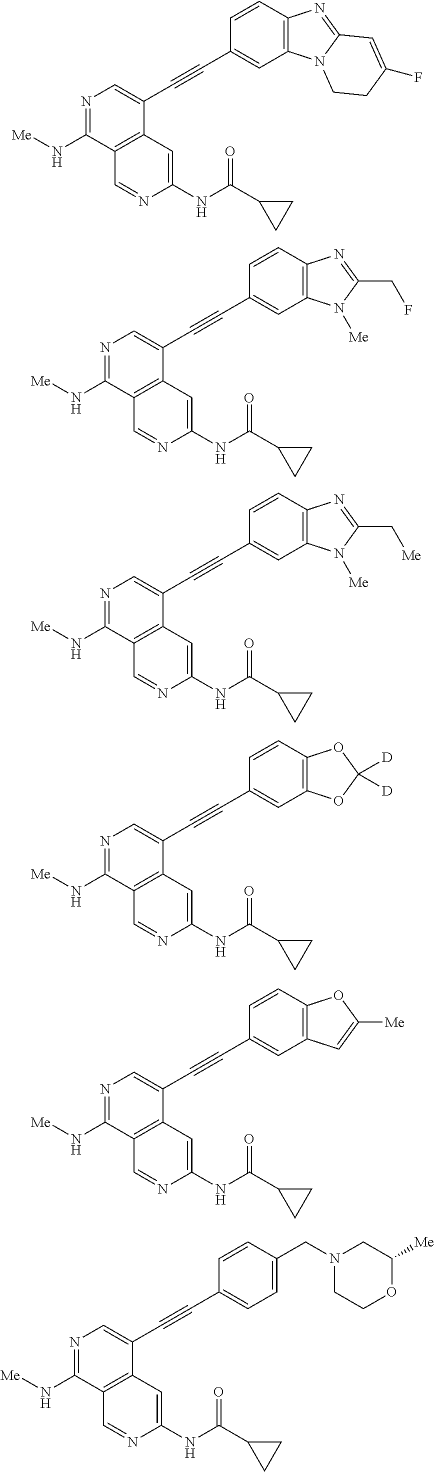

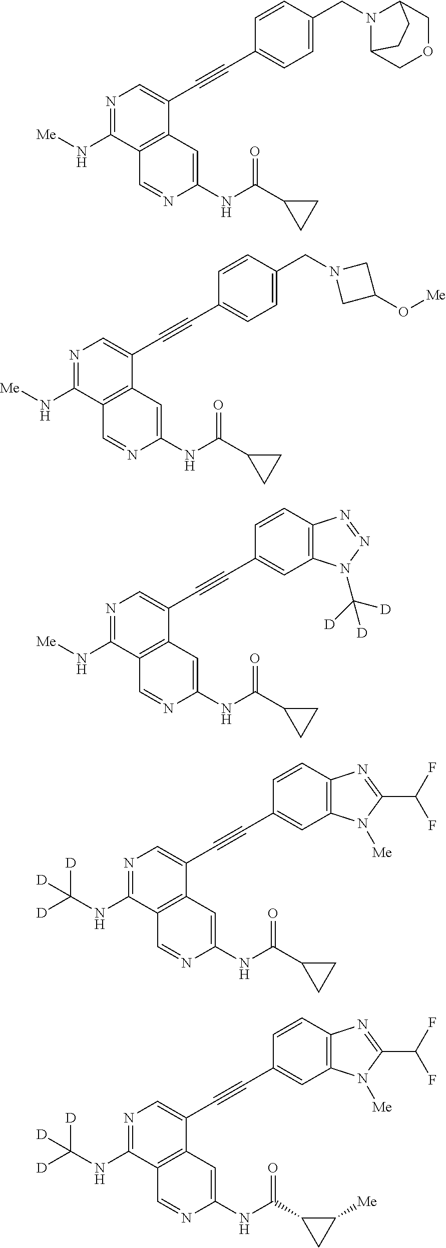

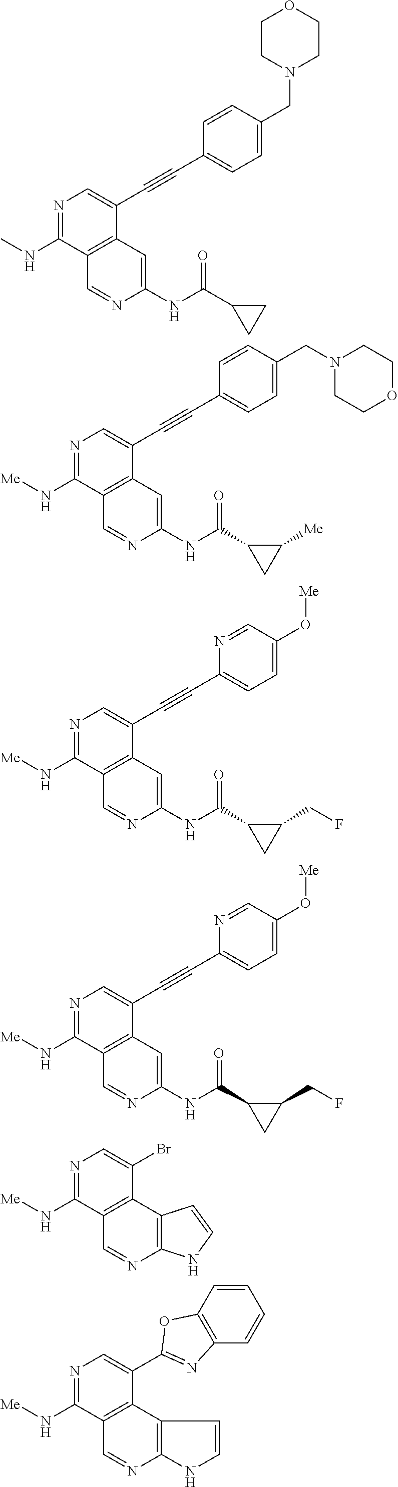

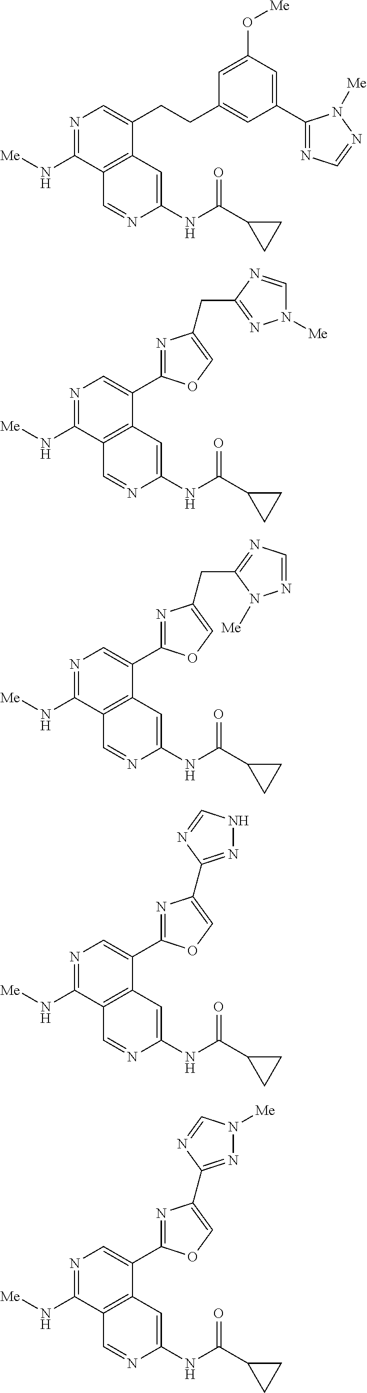

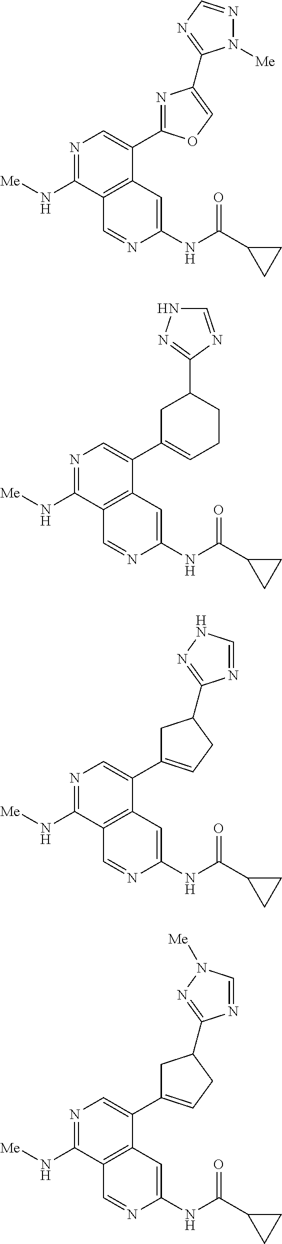 [SMILES: CNc1ncc(-c2nc(-c3ncnn3C)co2)c2cc(NC(=O)C3CC3)ncc12 ; CNc1ncc(C2=CCCC(c3nc[nH]n3)C2)c2cc(NC(=O)C3CC3)ncc12 ; CNc1ncc(C2=CCC(c3nc[nH]n3)C2)c2cc(NC(=O)C3CC3)ncc12 ; CNc1ncc(C2=CCC(c3ncn(C)n3)C2)c2cc(NC(=O)C3CC3)ncc12]

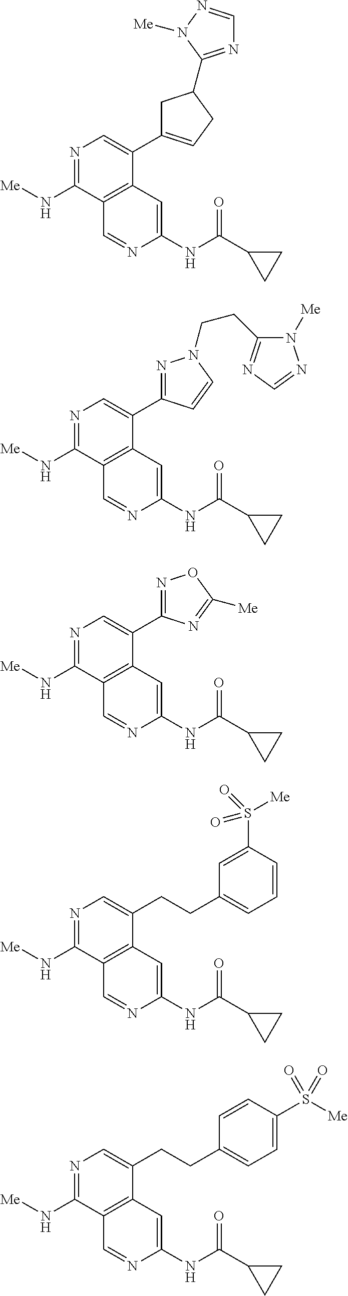

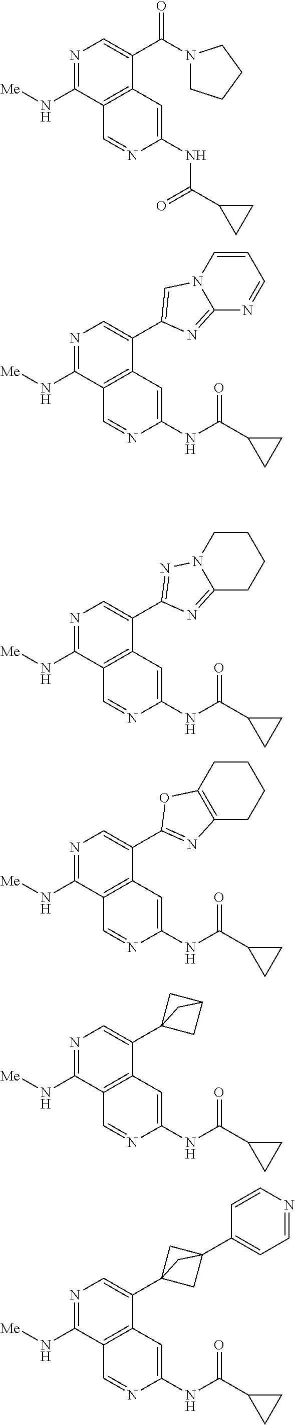

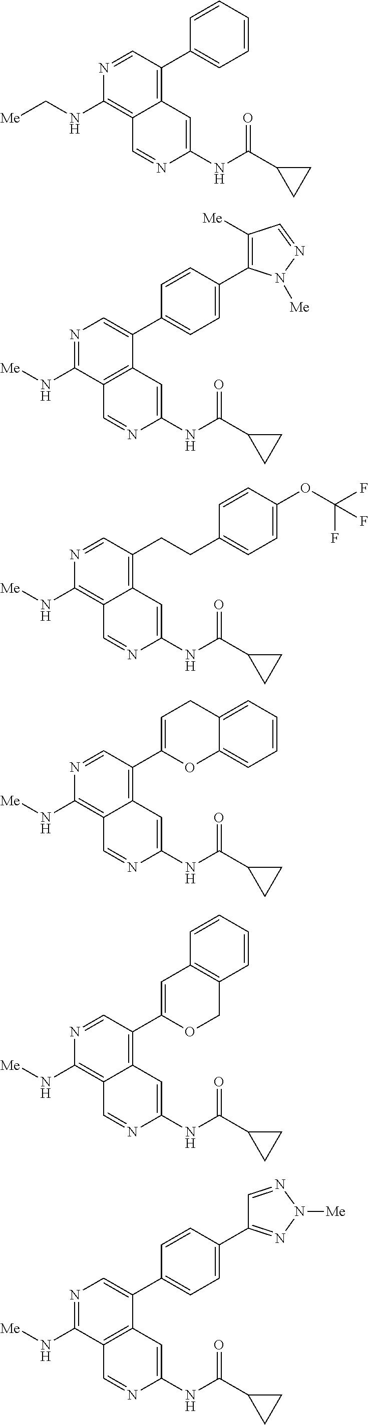

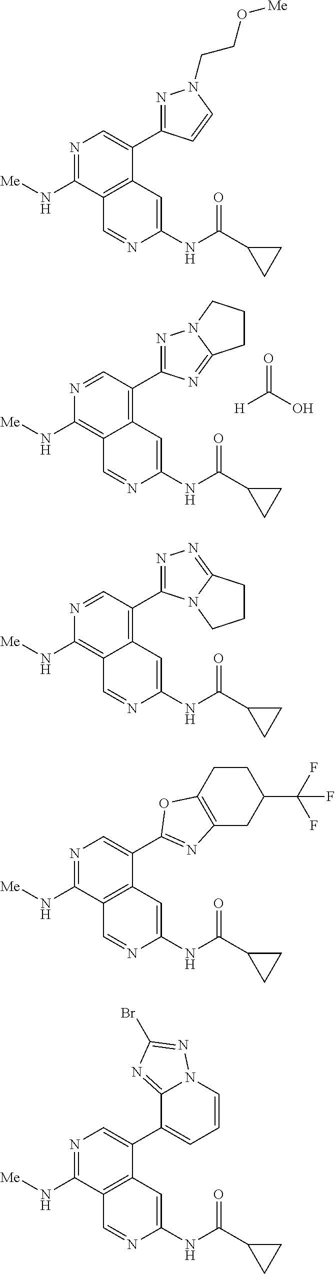

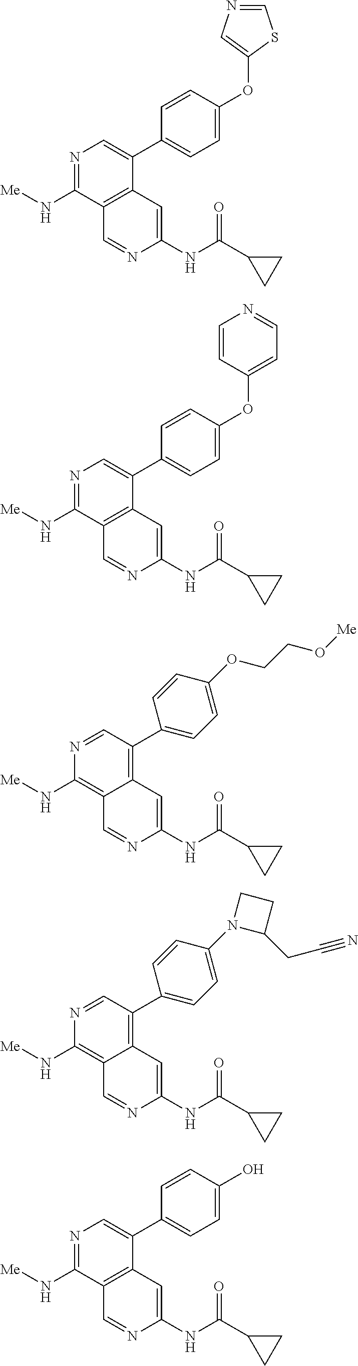

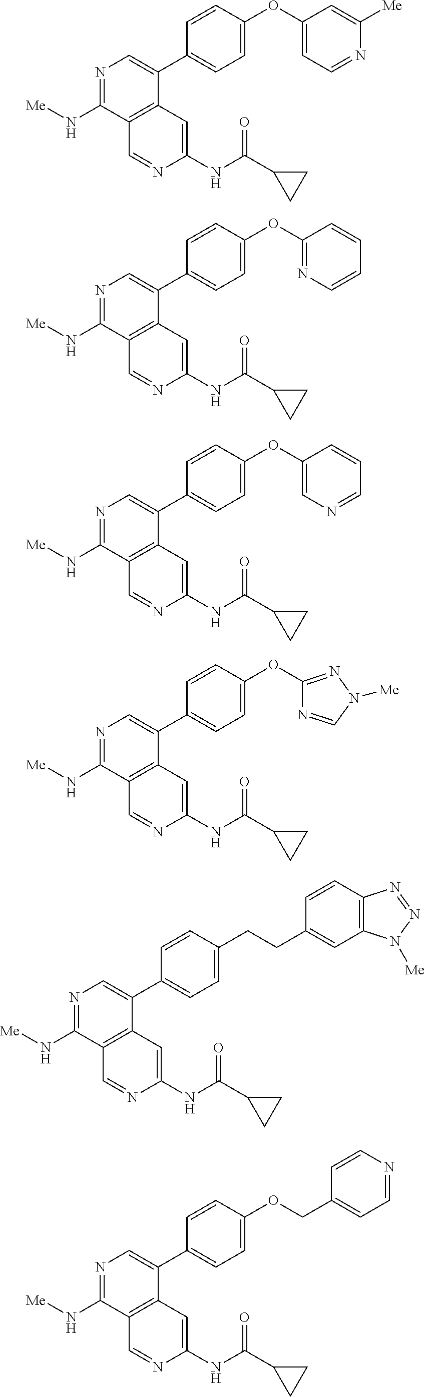

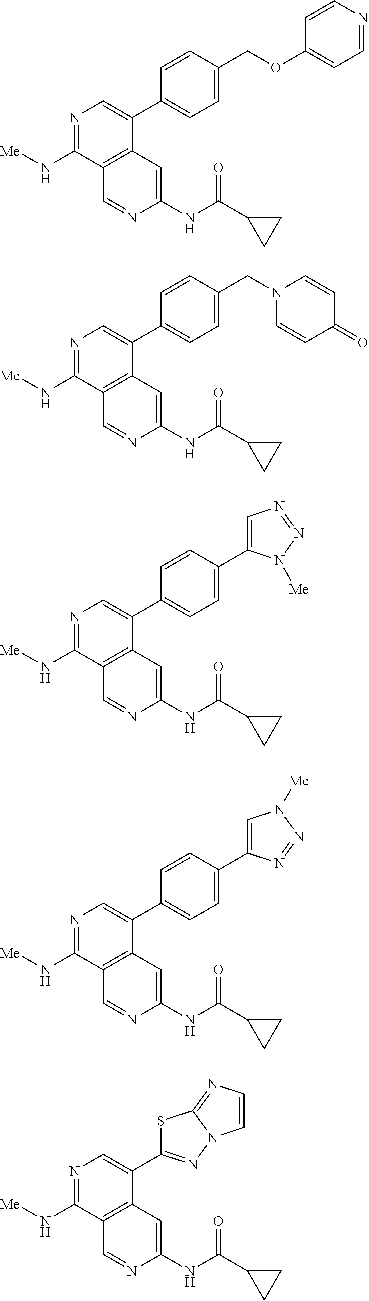

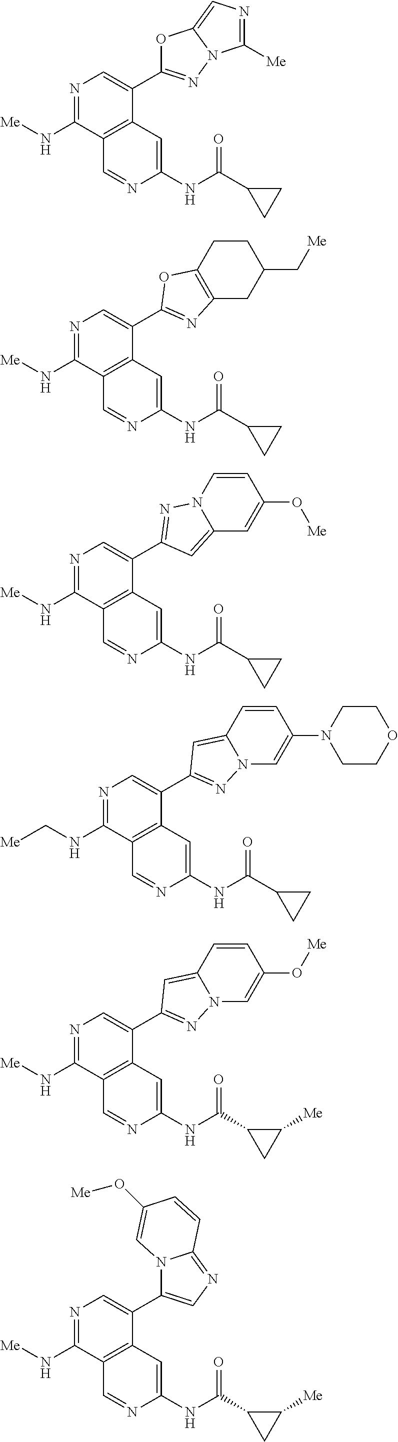

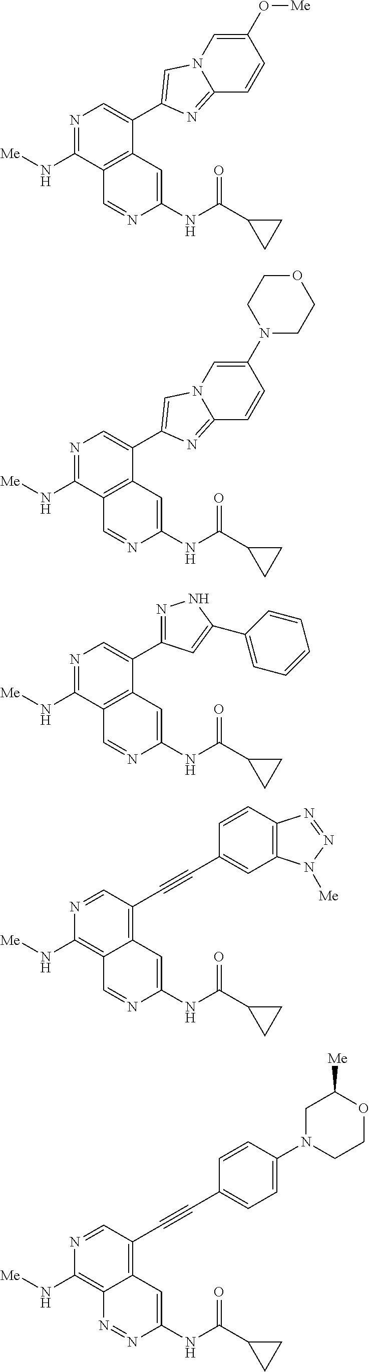

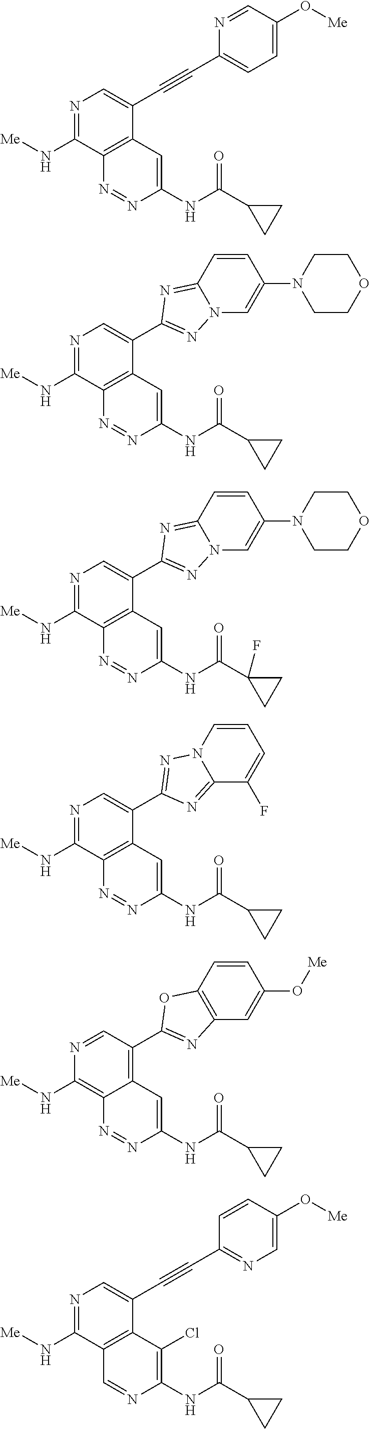

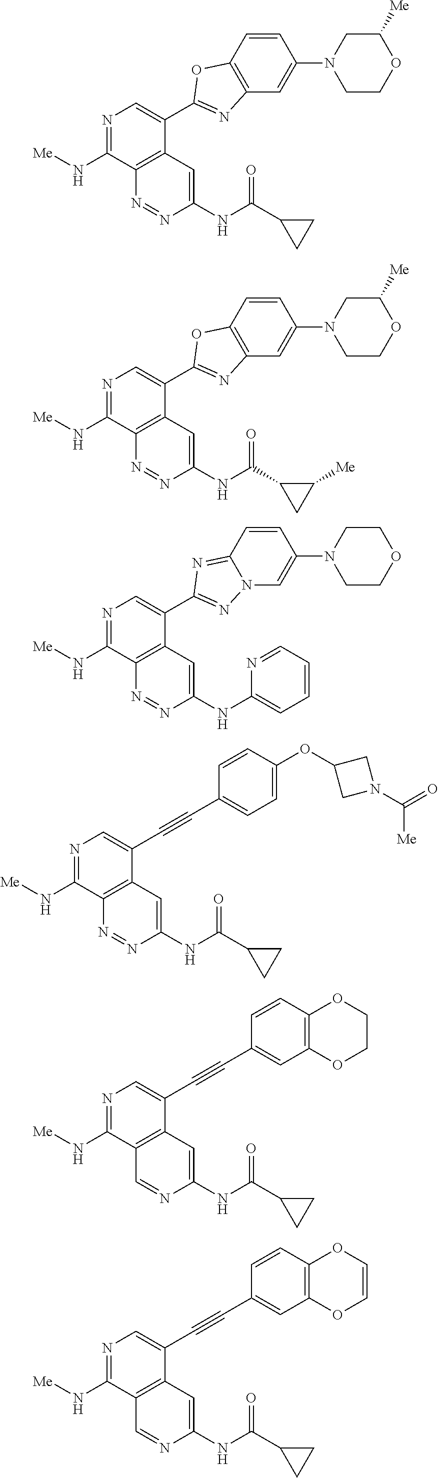

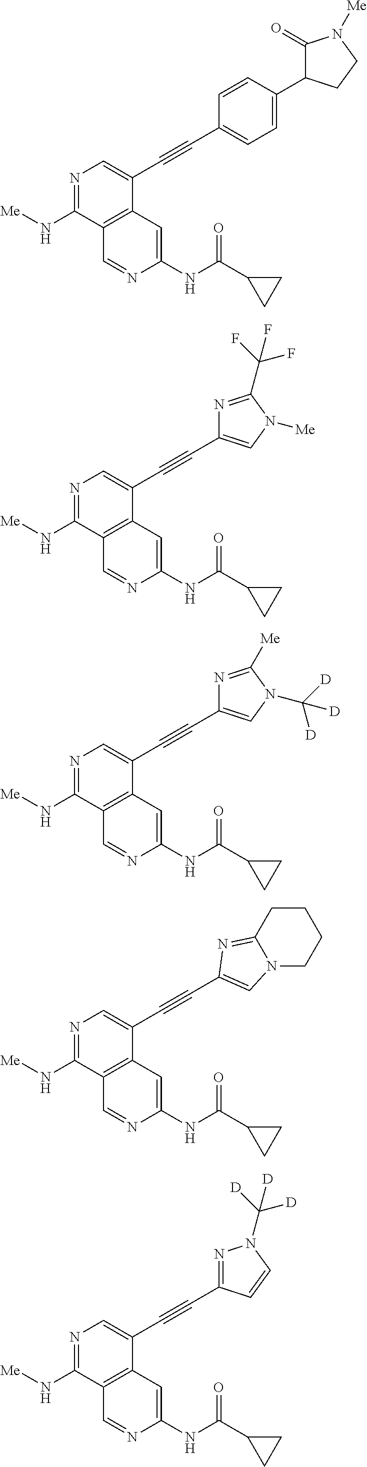

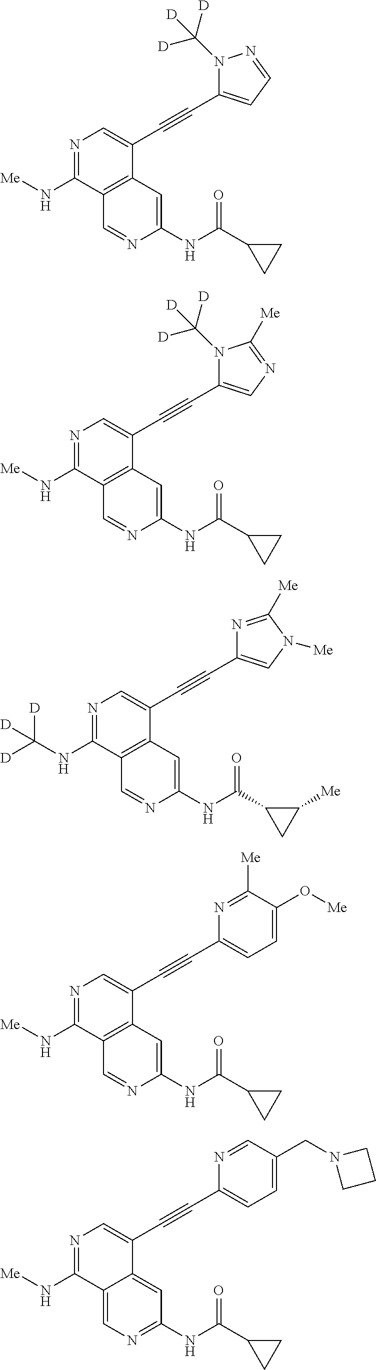

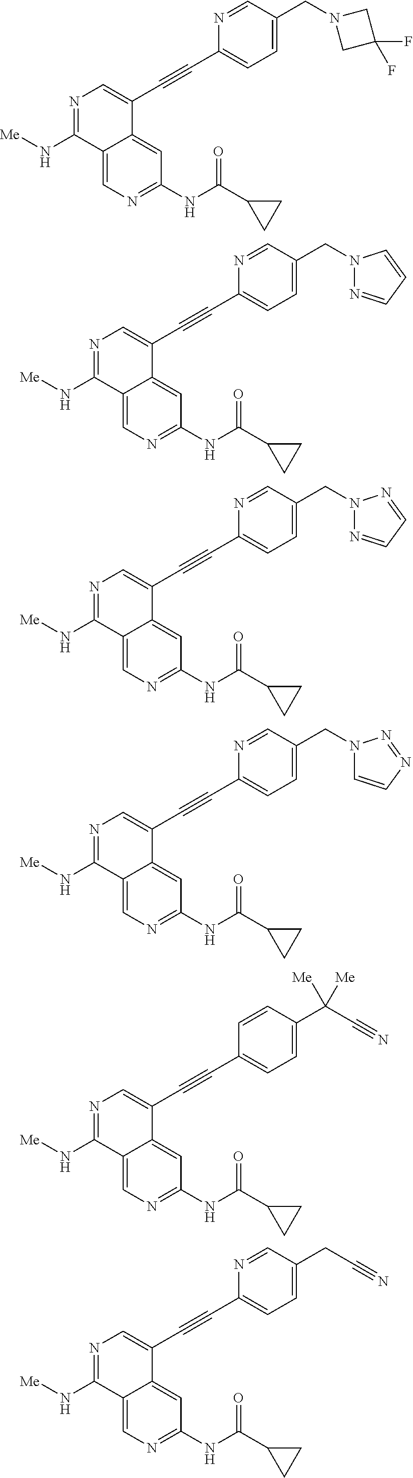

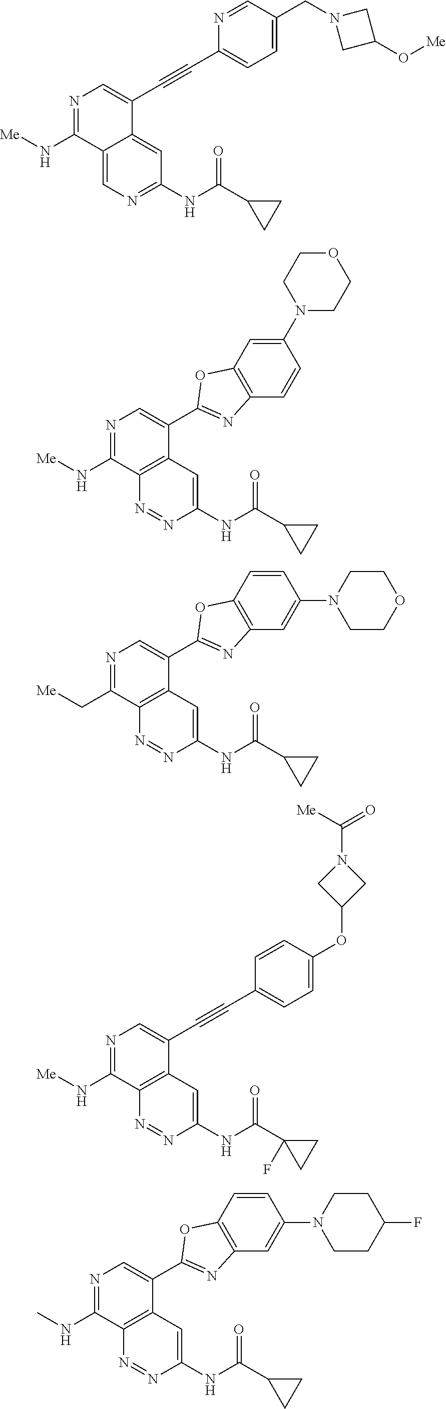

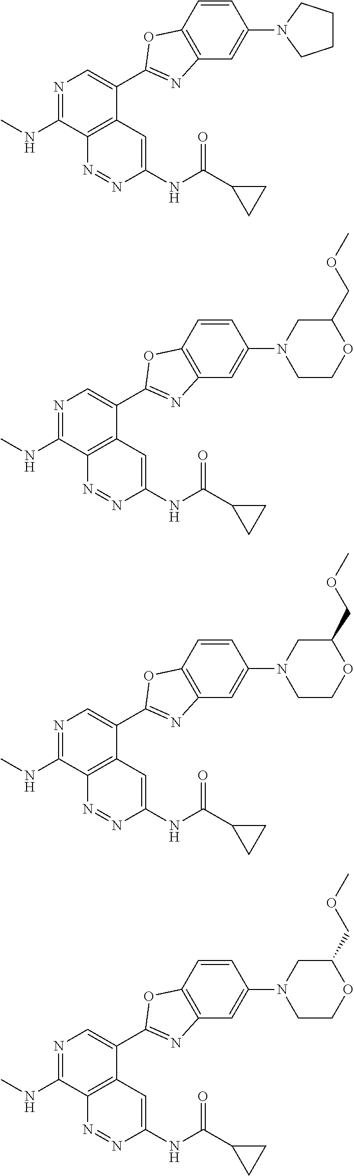

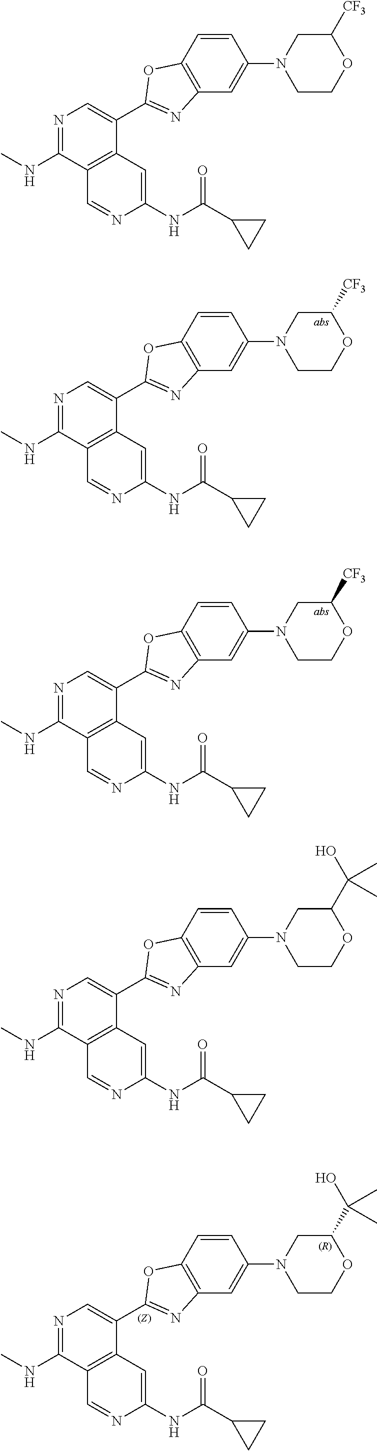

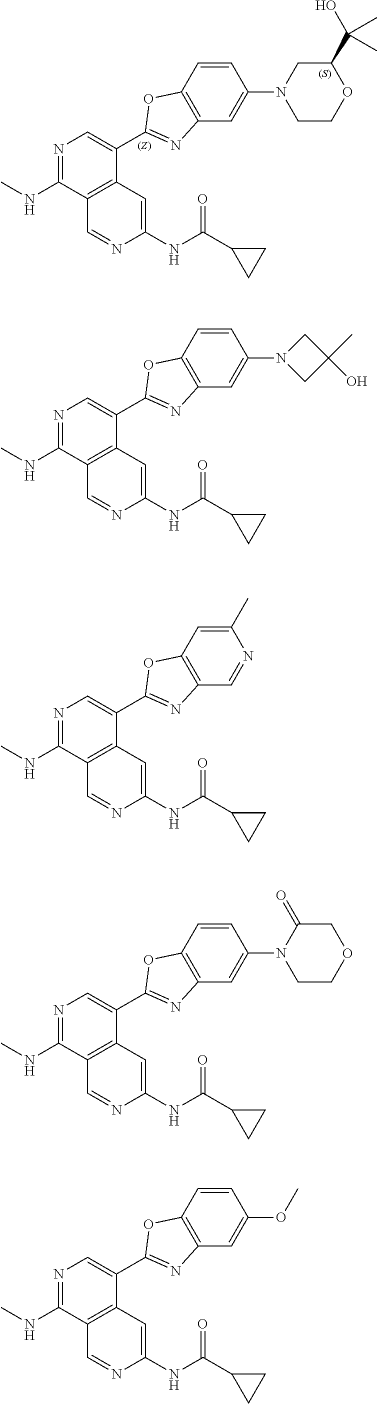

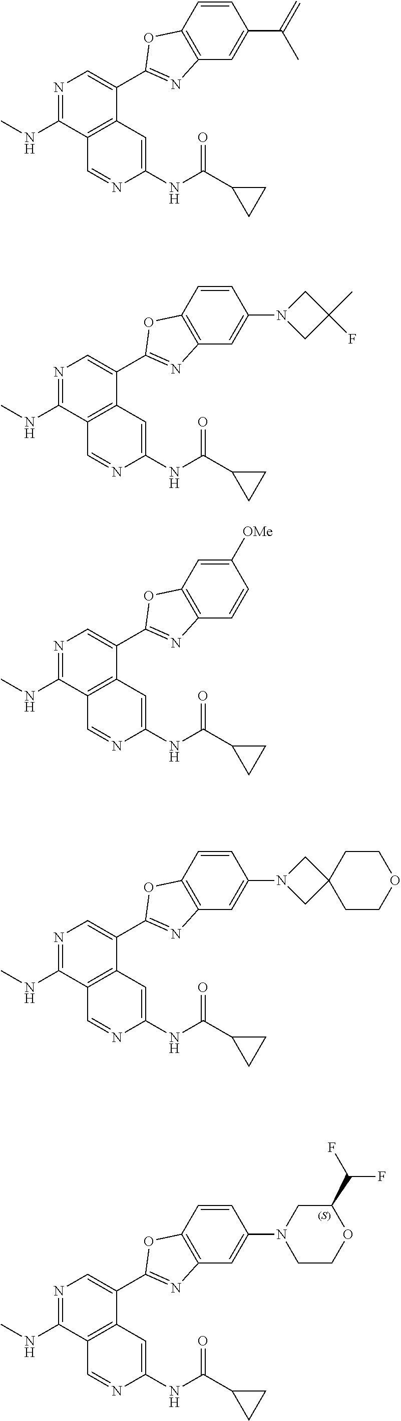

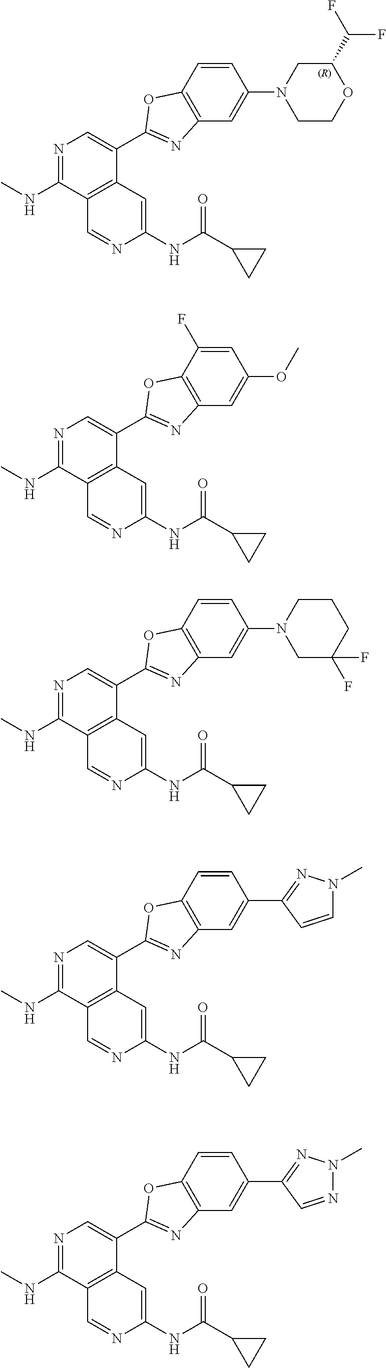

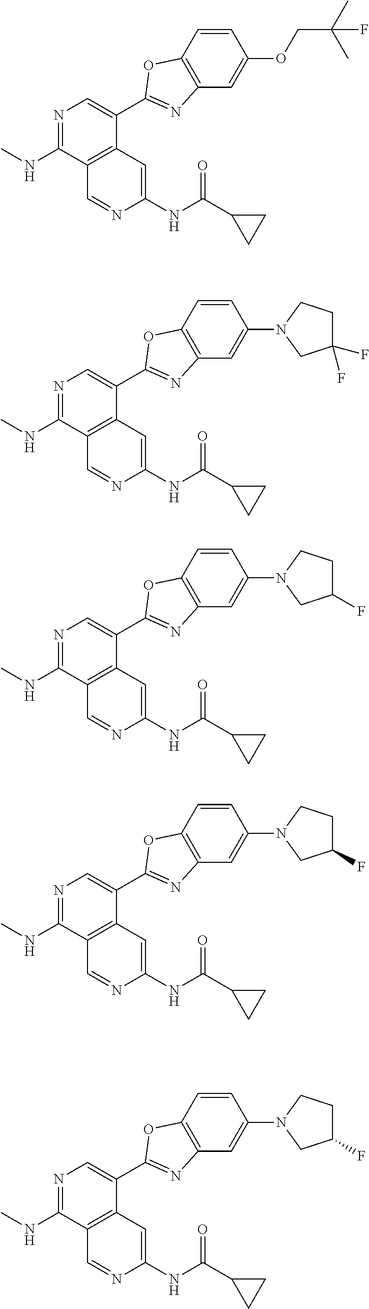

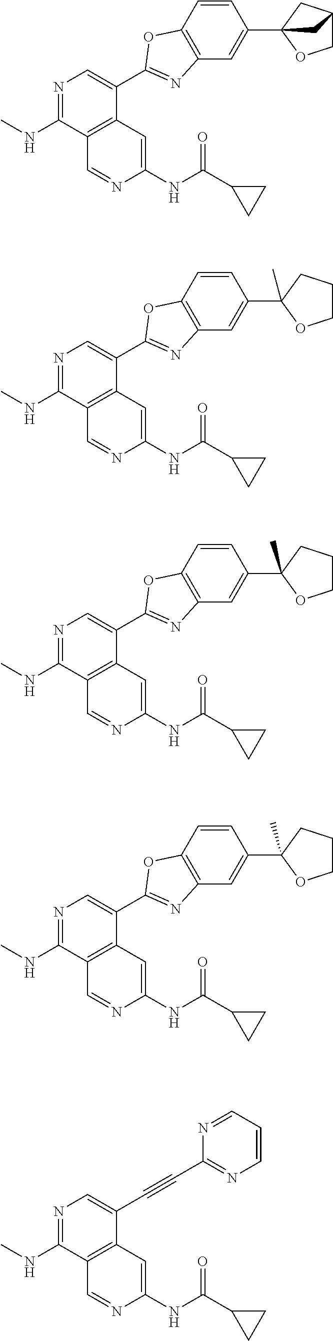 [SMILES: CNc1ncc(-c2nc3cc([C@]45C[C@H](CO4)C5)ccc3o2)c2cc(NC(=O)C3CC3)ncc12 ; CNc1ncc(-c2nc3cc(C4(C)CCCO4)ccc3o2)c2cc(NC(=O)C3CC3)ncc12 ; CNc1ncc(-c2nc3cc([C@]4(C)CCCO4)ccc3o2)c2cc(NC(=O)C3CC3)ncc12 ; CNc1ncc(-c2nc3cc([C@@]4(C)CCCO4)ccc3o2)c2cc(NC(=O)C3CC3)ncc12 ; CNc1ncc(C#Cc2ncccn2)c2cc(NC(=O)C3CC3)ncc12]

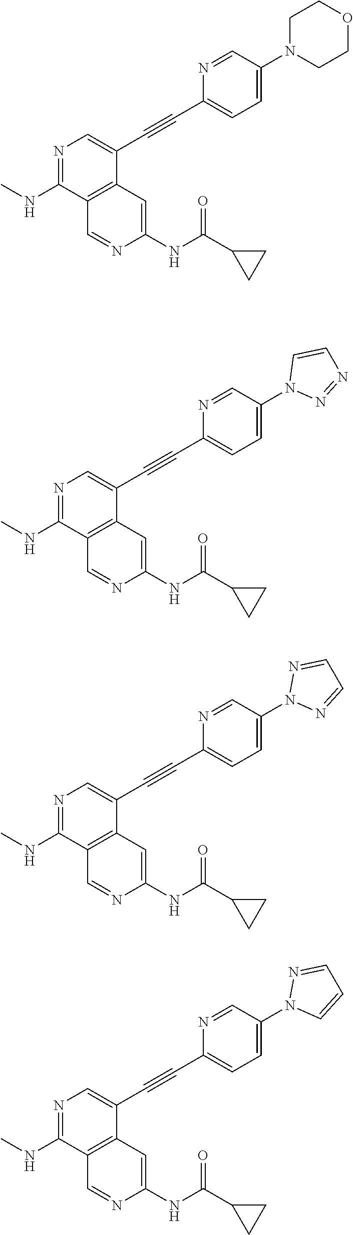 [SMILES: CNc1ncc(C#Cc2ccc(N3CCOCC3)cn2)c2cc(NC(=O)C3CC3)ncc12 ; CNc1ncc(C#Cc2ccc(-n3ccnn3)cn2)c2cc(NC(=O)C3CC3)ncc12 ; CNc1ncc(C#Cc2ccc(-n3nccn3)cn2)c2cc(NC(=O)C3CC3)ncc12 ; CNc1ncc(C#Cc2ccc(-n3cccn3)cn2)c2cc(NC(=O)C3CC3)ncc12]

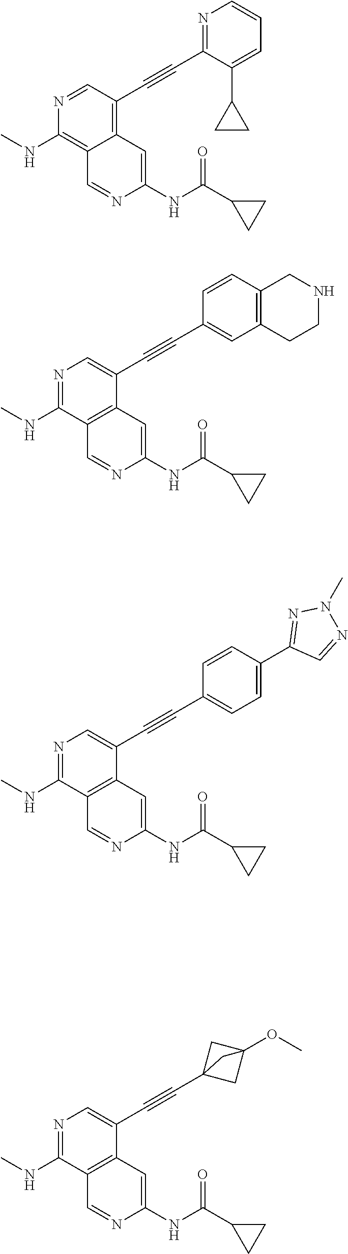

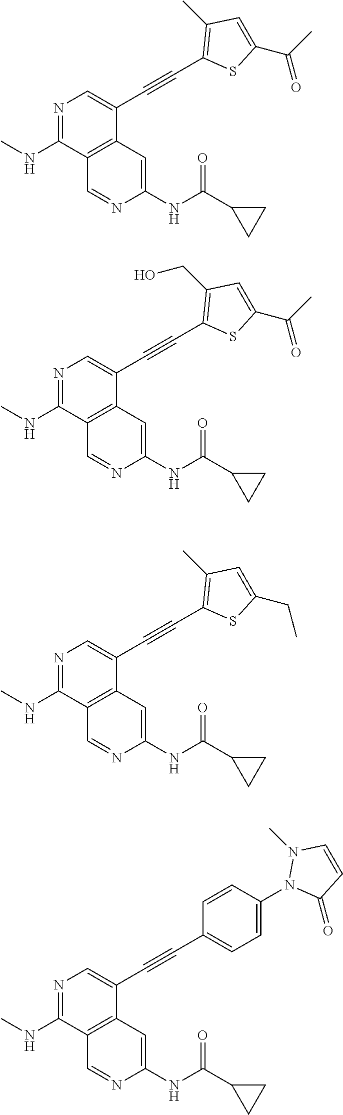

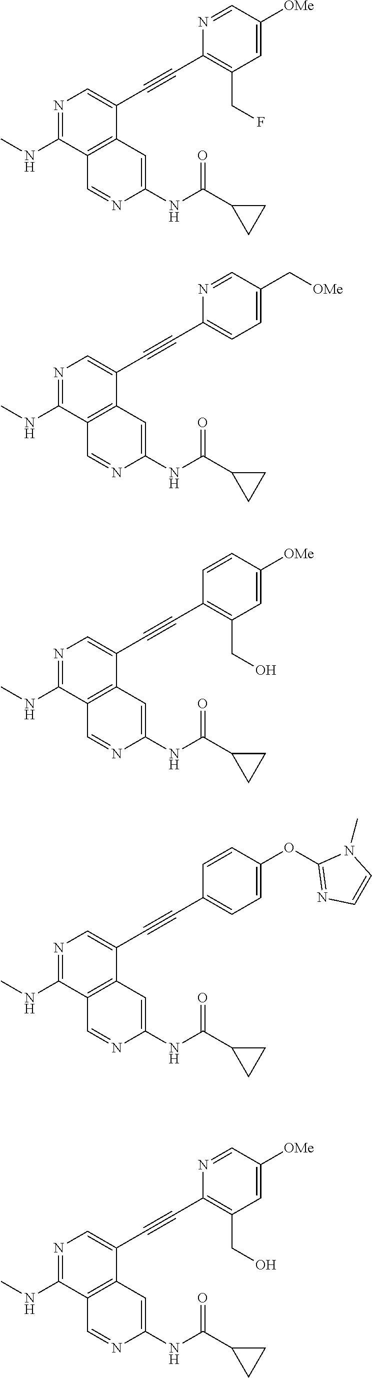

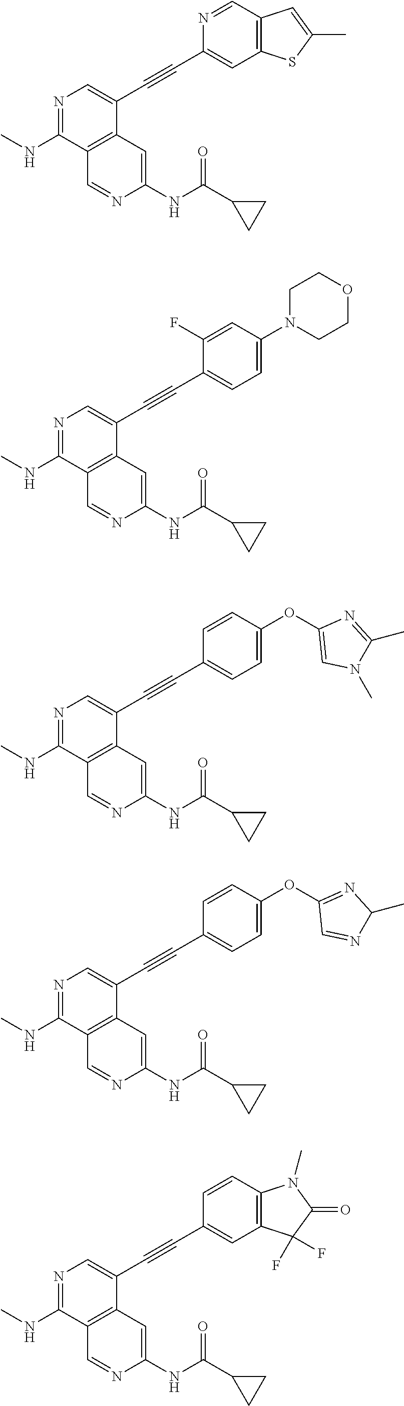

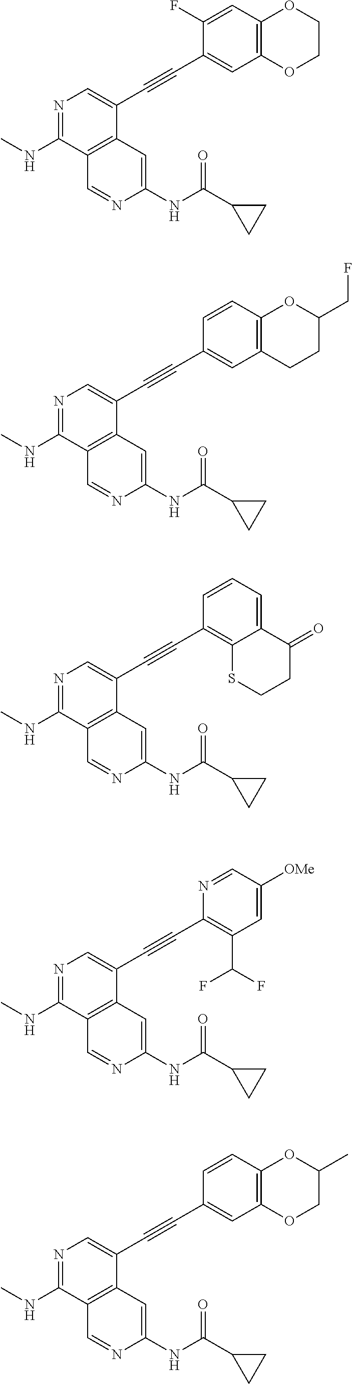

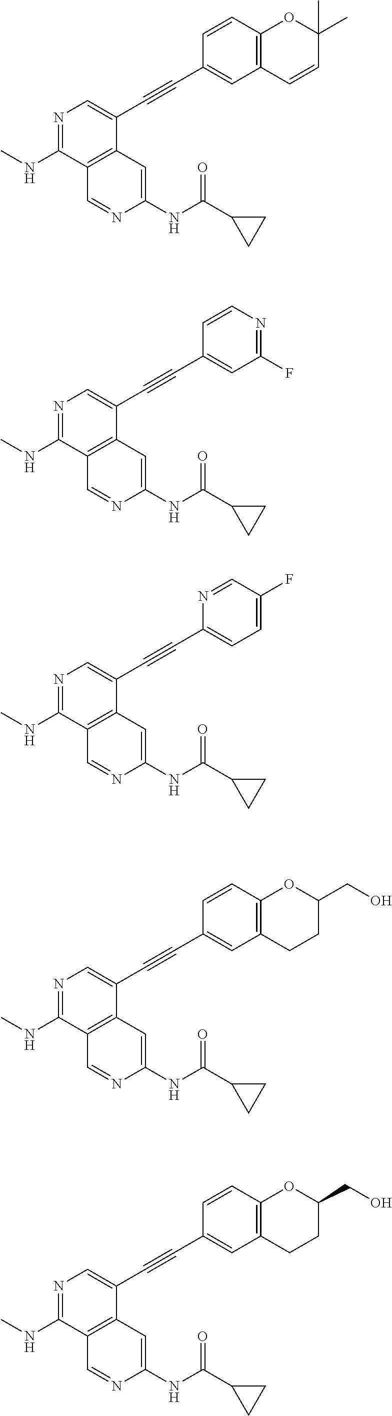

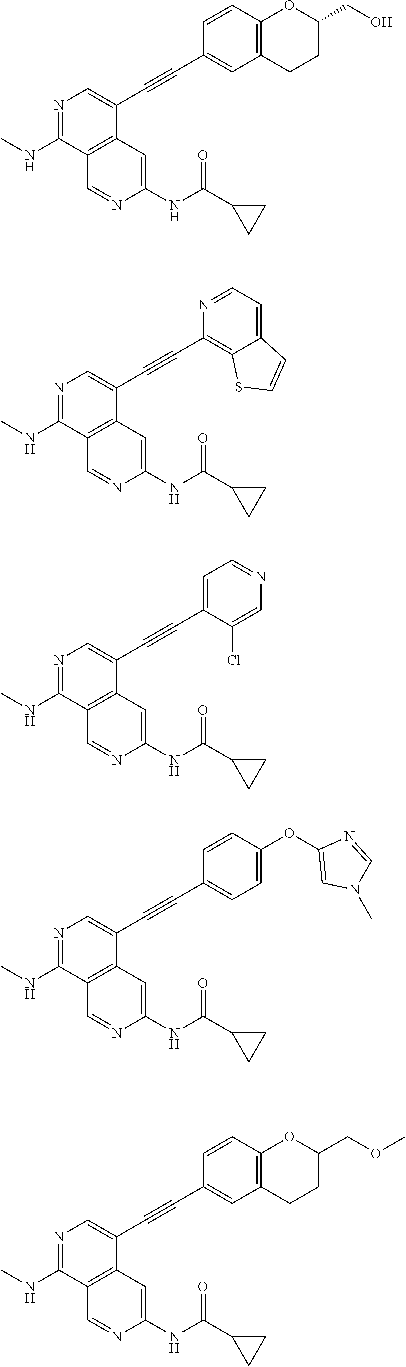

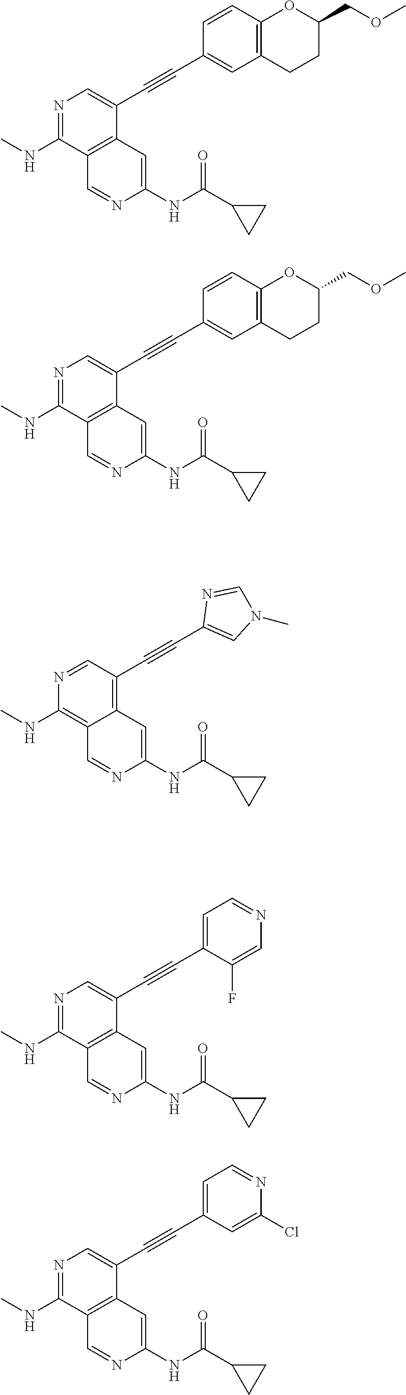

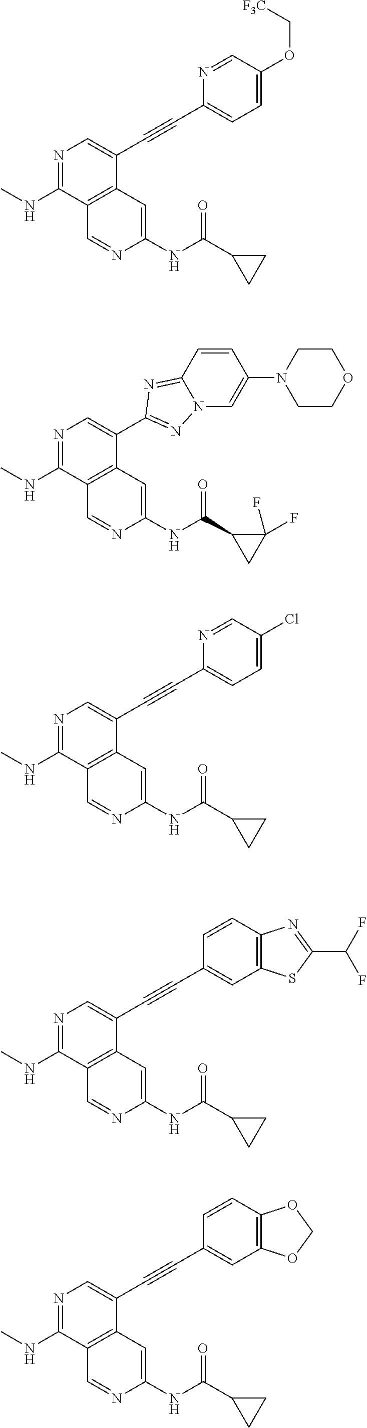 [SMILES: CNc1ncc(C#Cc2ccc(OCC(F)(F)F)cn2)c2cc(NC(=O)C3CC3)ncc12 ; CNc1ncc(-c2nc3ccc(N4CCOCC4)cn3n2)c2cc(NC(=O)[C@@H]3CC3(F)F)ncc12 ; CNc1ncc(C#Cc2ccc(Cl)cn2)c2cc(NC(=O)C3CC3)ncc12 ; CNc1ncc(C#Cc2ccc3nc(C(F)F)sc3c2)c2cc(NC(=O)C3CC3)ncc12 ; CNc1ncc(C#Cc2ccc3c(c2)OCO3)c2cc(NC(=O)C3CC3)ncc12]

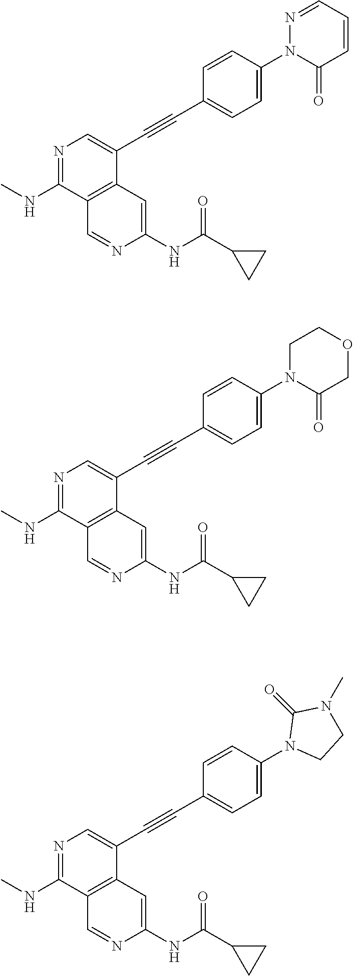

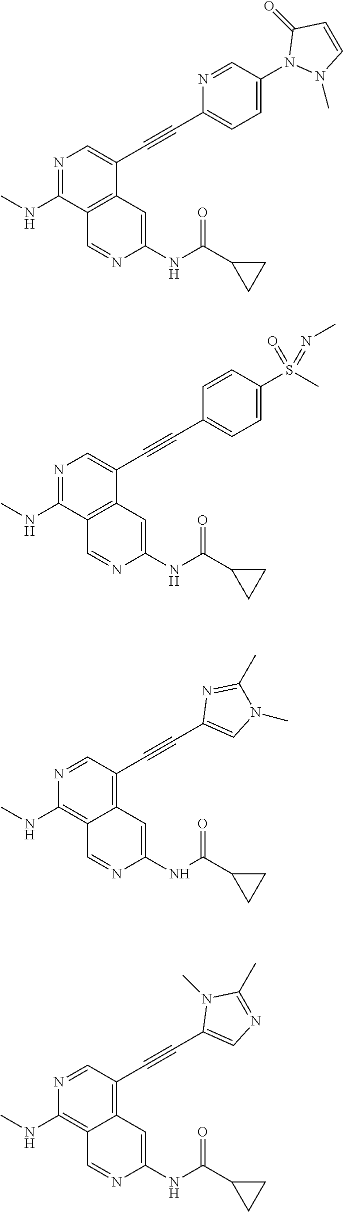

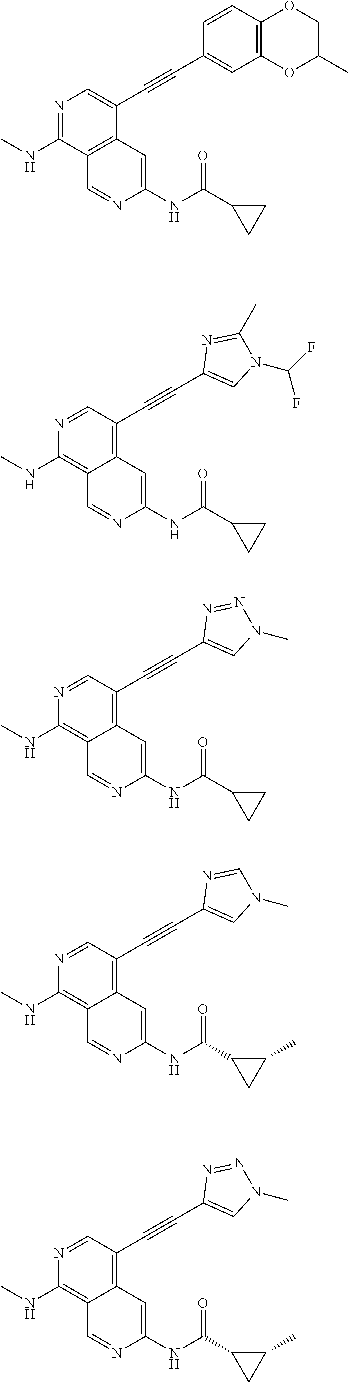

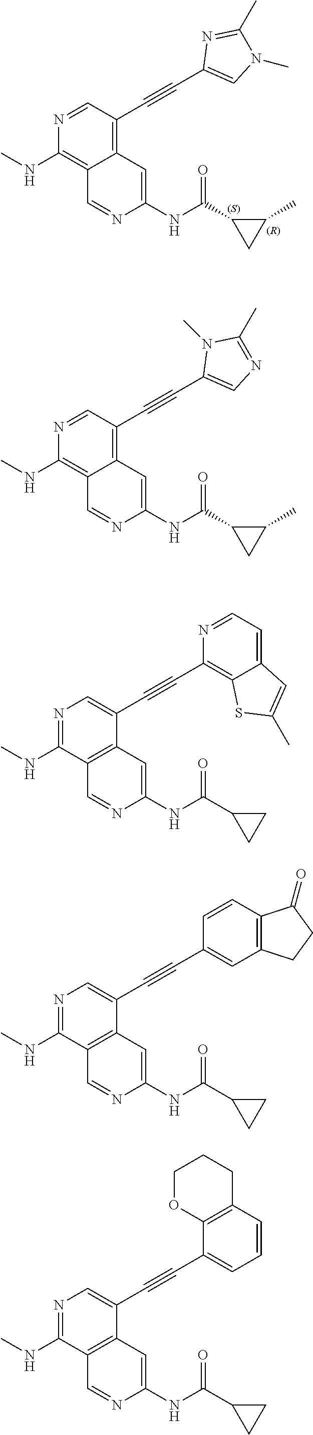

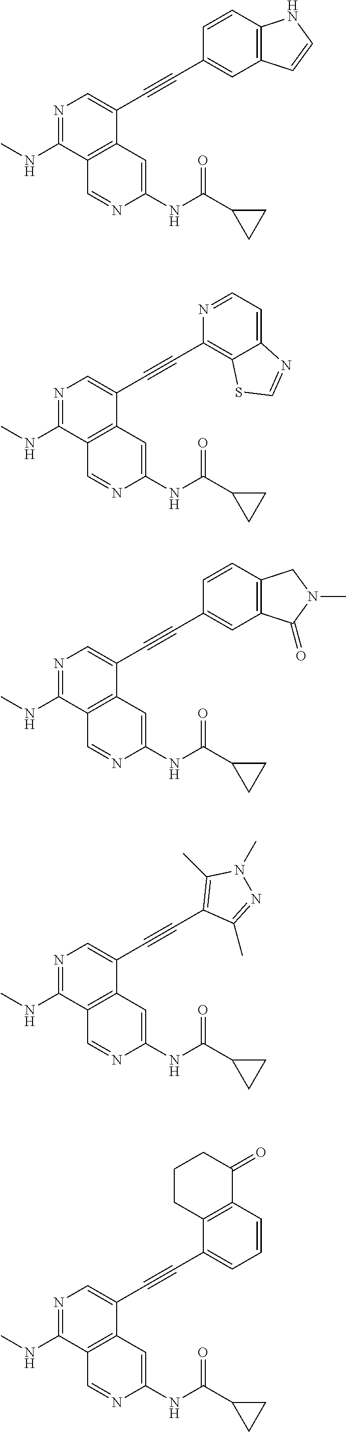

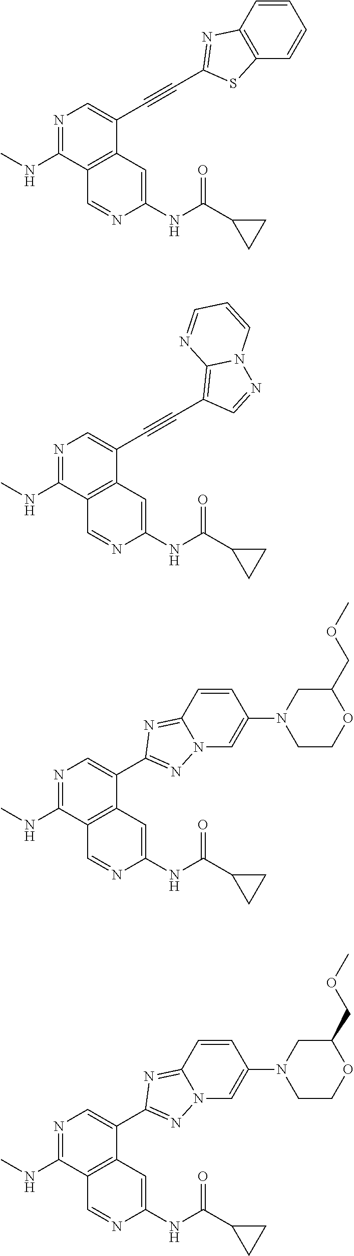

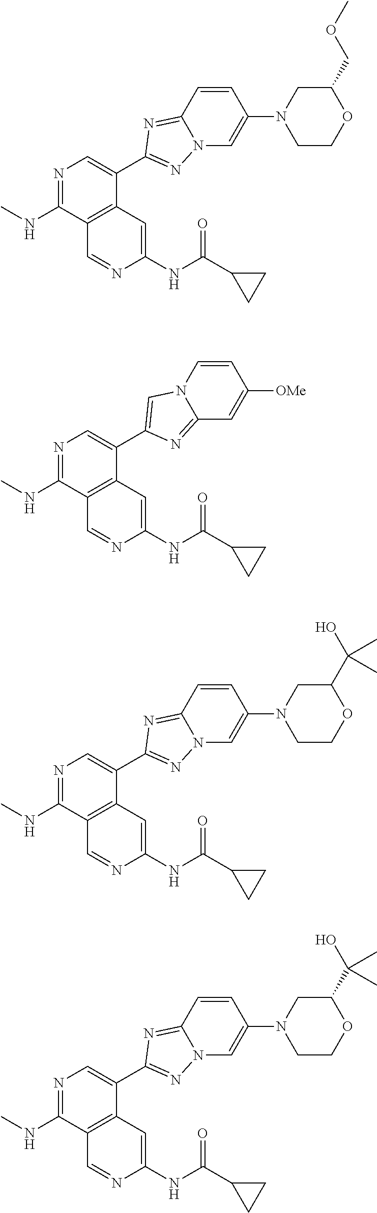

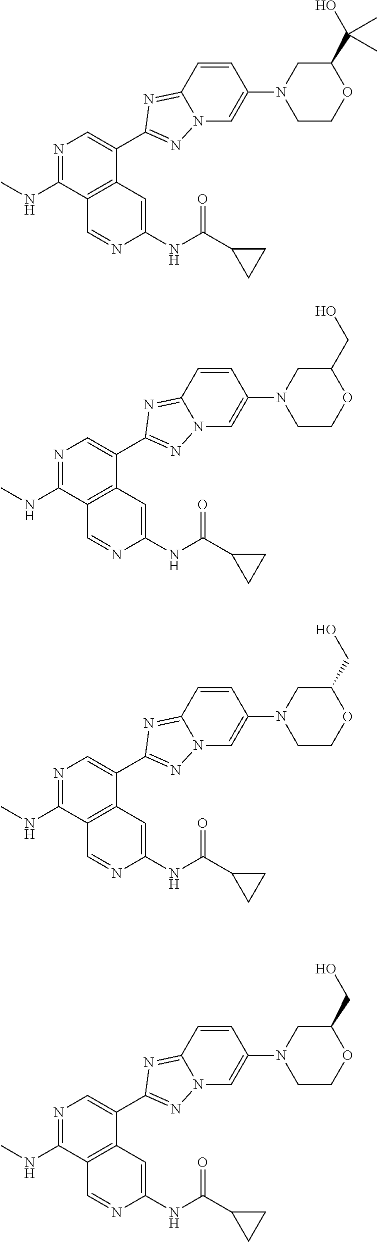

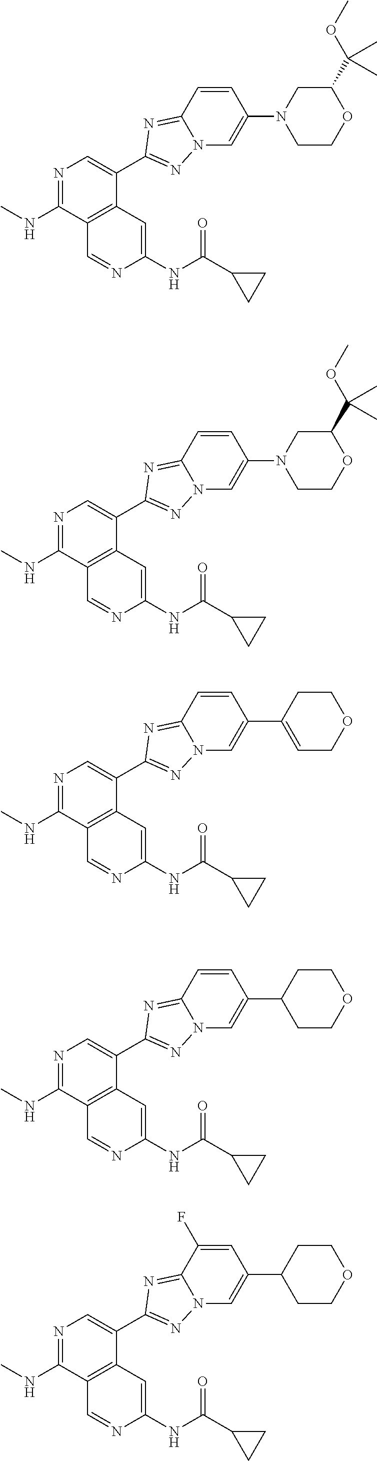

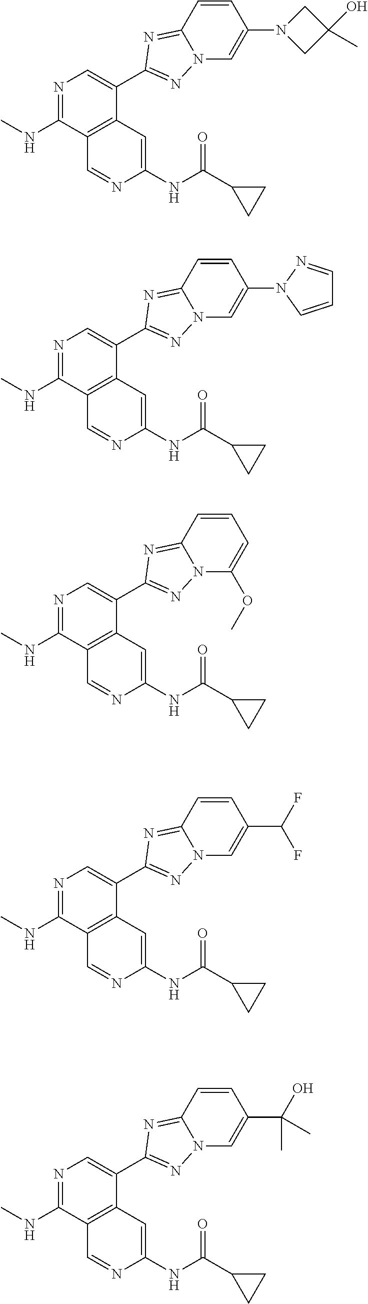

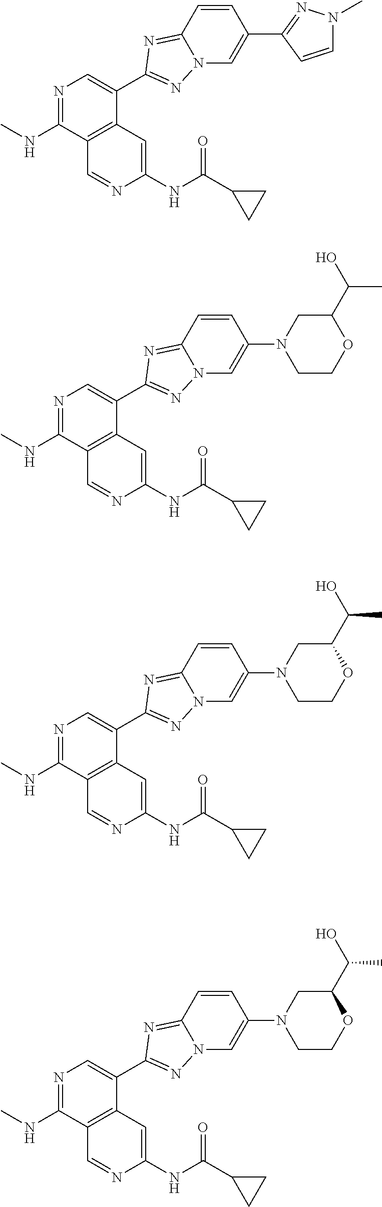

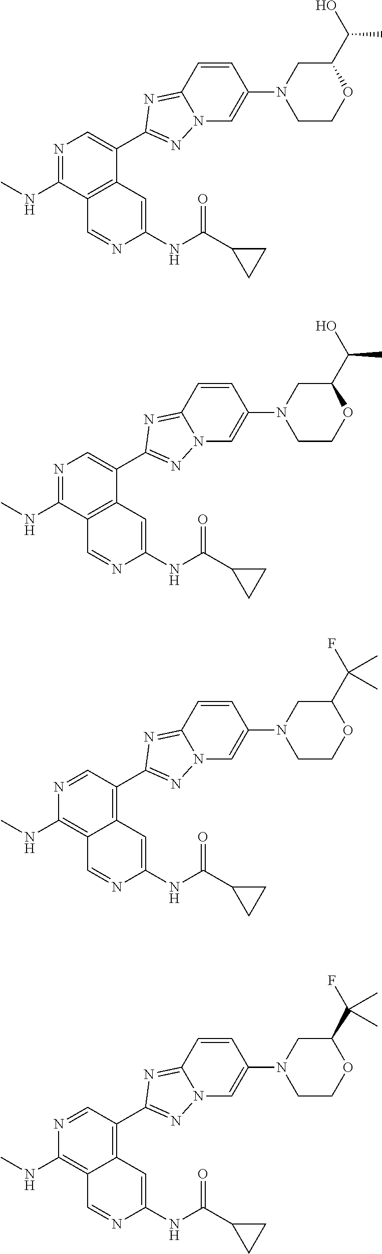

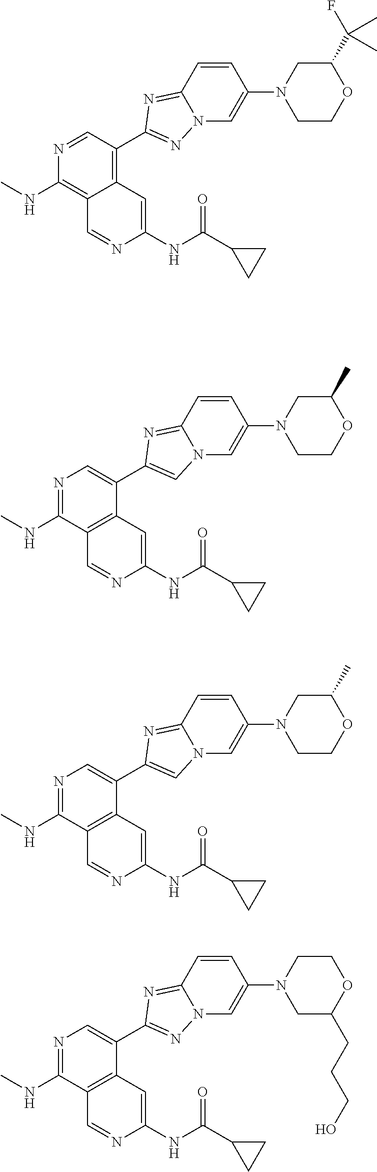

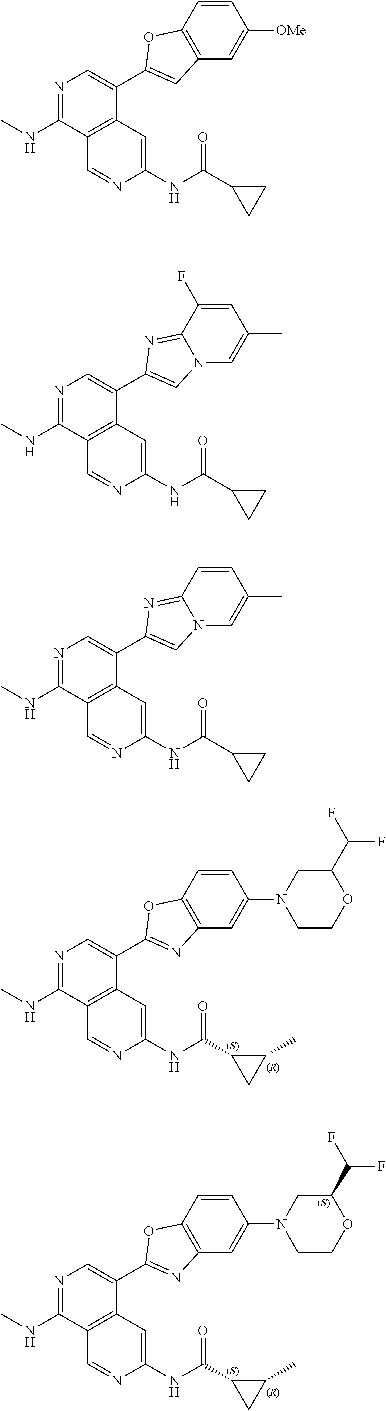

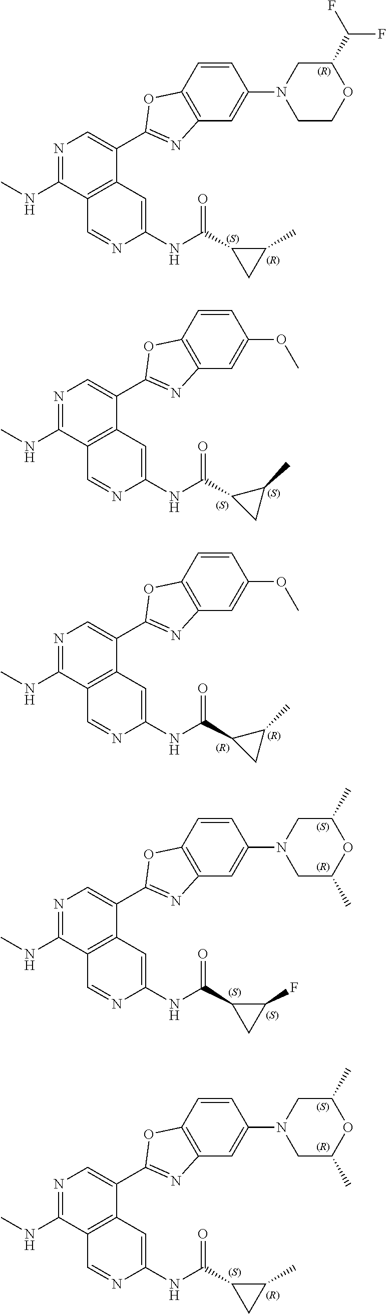

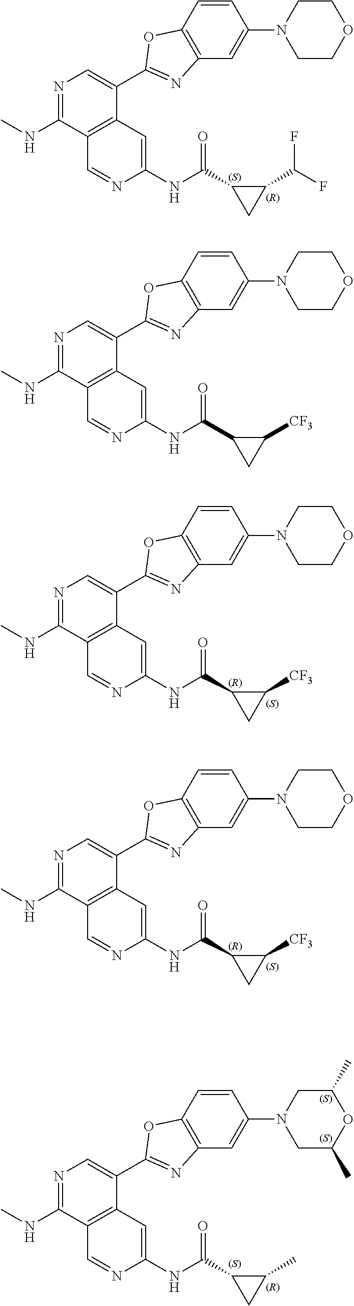

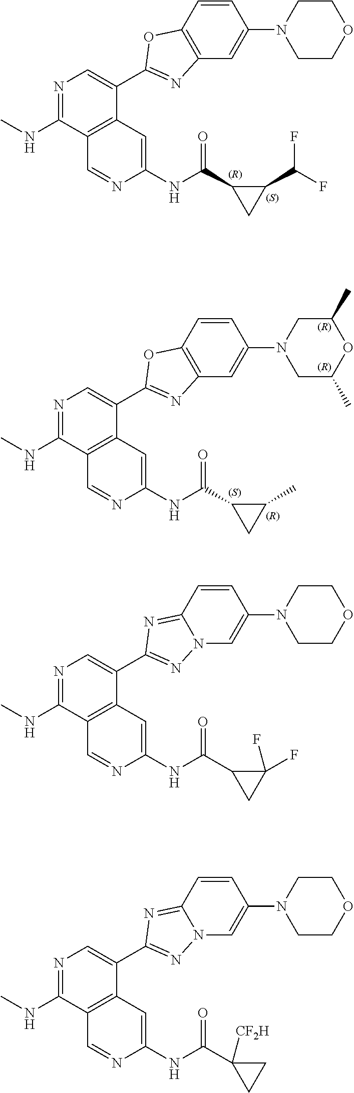

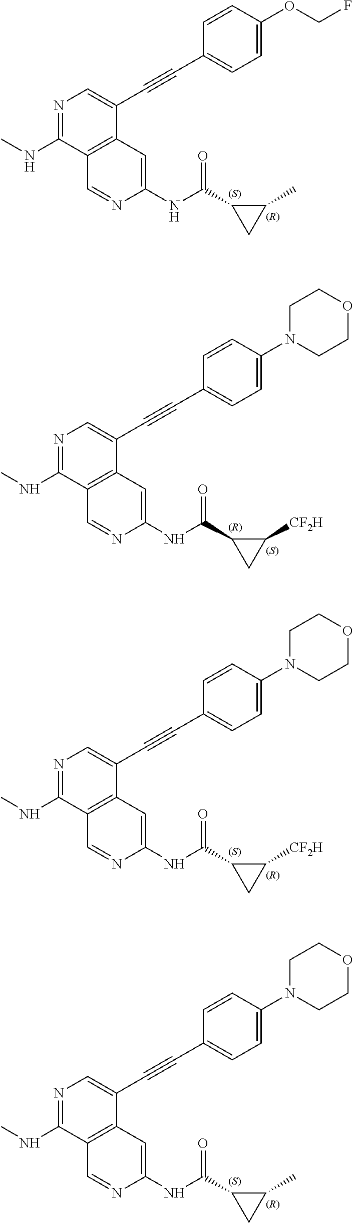

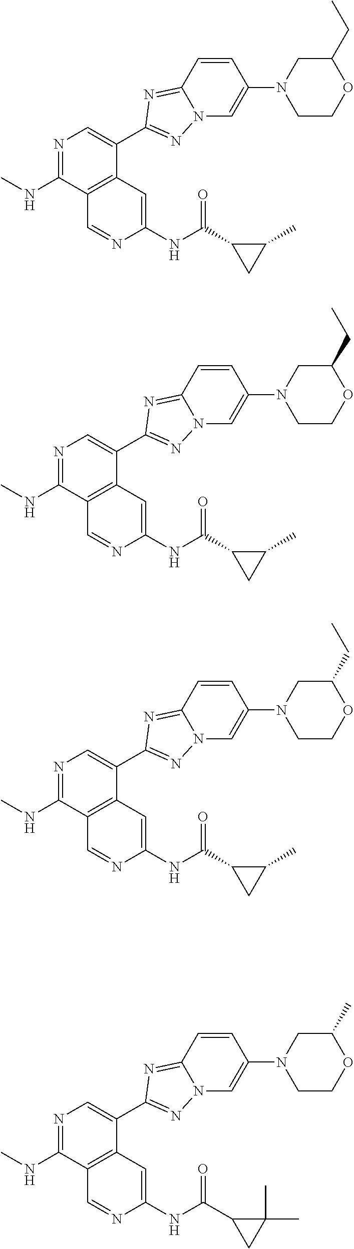

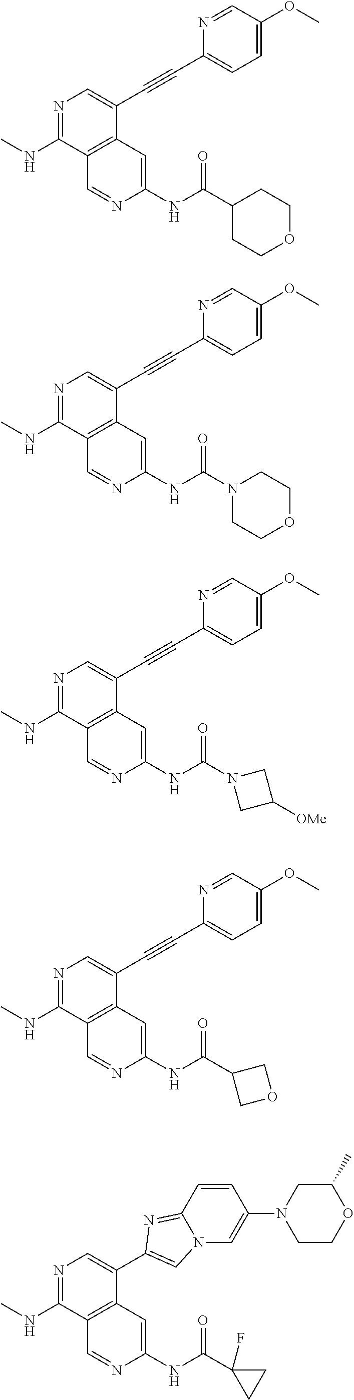

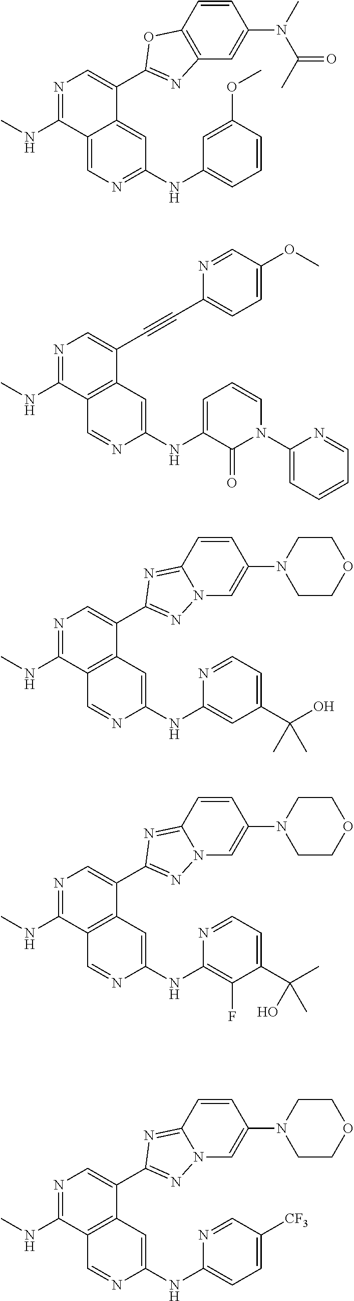

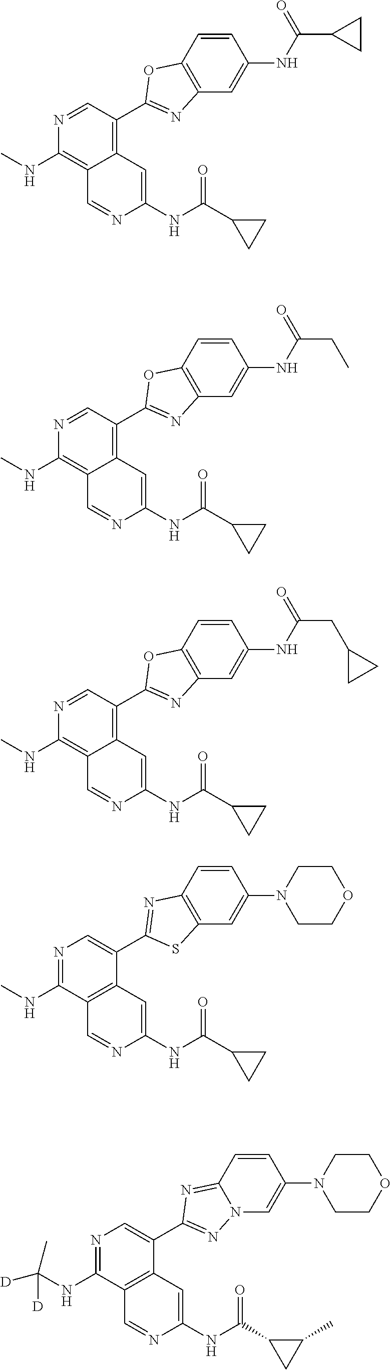

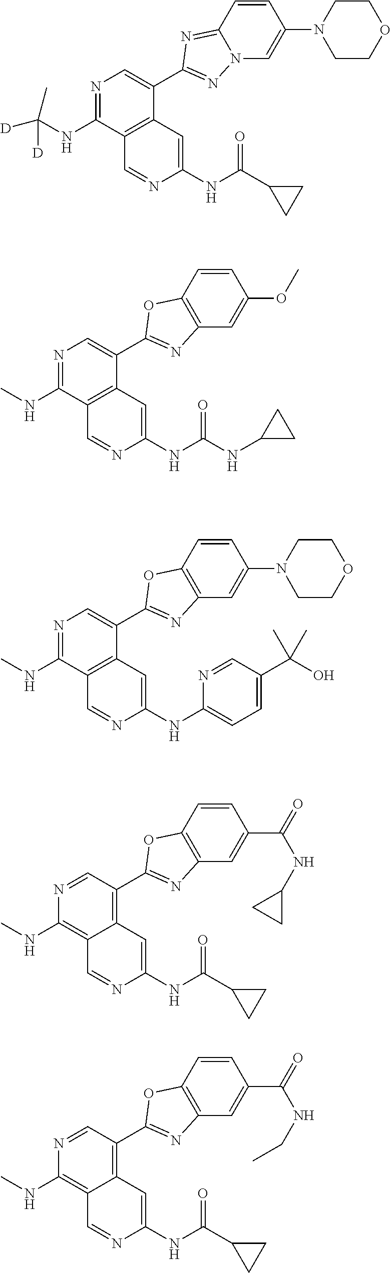

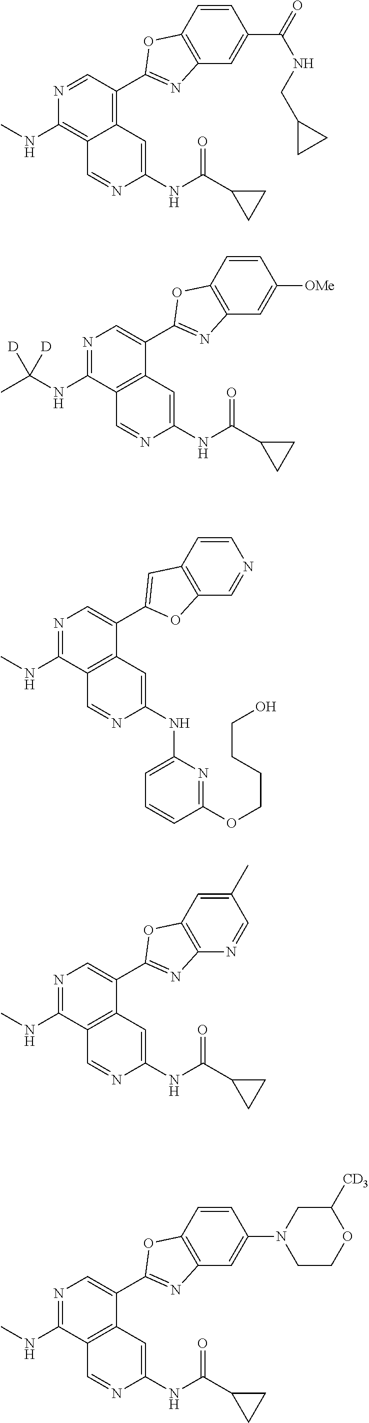

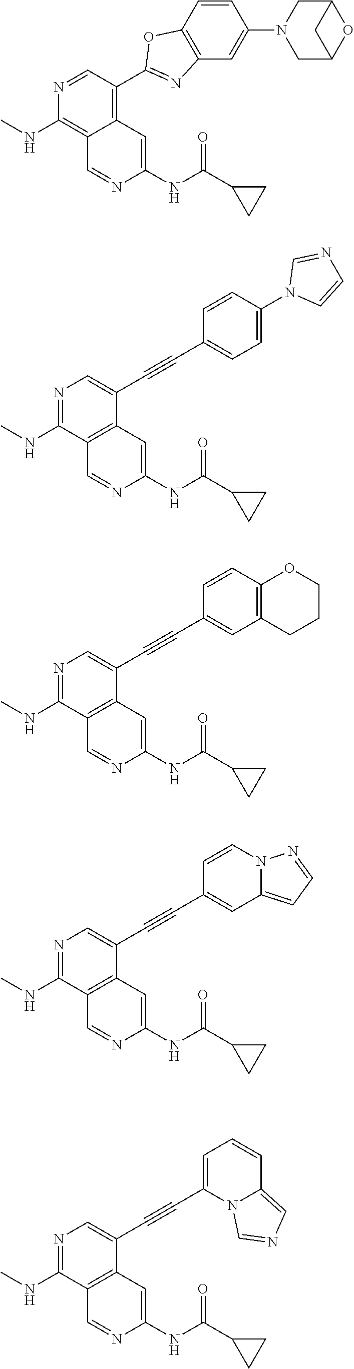 [SMILES: CNc1ncc(-c2nc3cc(N4CC5CC(C4)O5)ccc3o2)c2cc(NC(=O)C3CC3)ncc12 ; CNc1ncc(C#Cc2ccc(-n3ccnc3)cc2)c2cc(NC(=O)C3CC3)ncc12 ; CNc1ncc(C#Cc2ccc3c(c2)CCCO3)c2cc(NC(=O)C3CC3)ncc12 ; CNc1ncc(C#Cc2ccn3nccc3c2)c2cc(NC(=O)C3CC3)ncc12 ; CNc1ncc(C#Cc2cccc3cncn23)c2cc(NC(=O)C3CC3)ncc12]

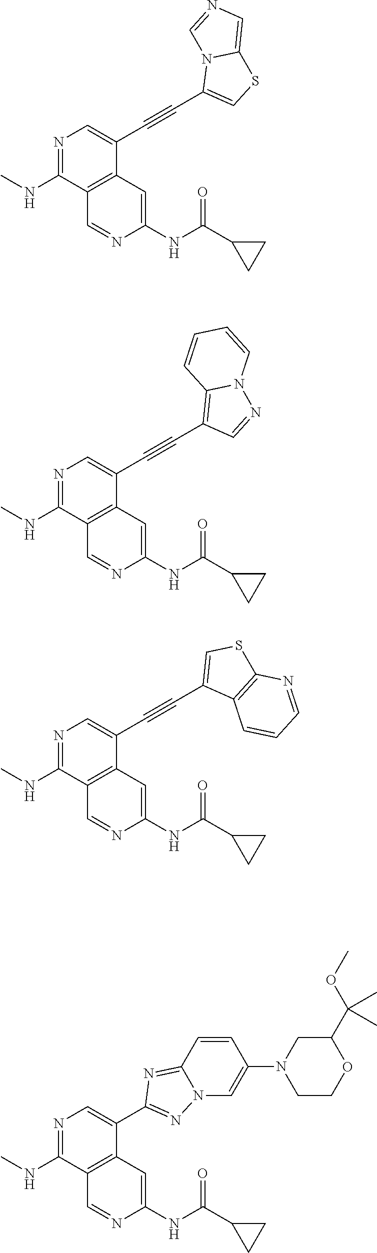

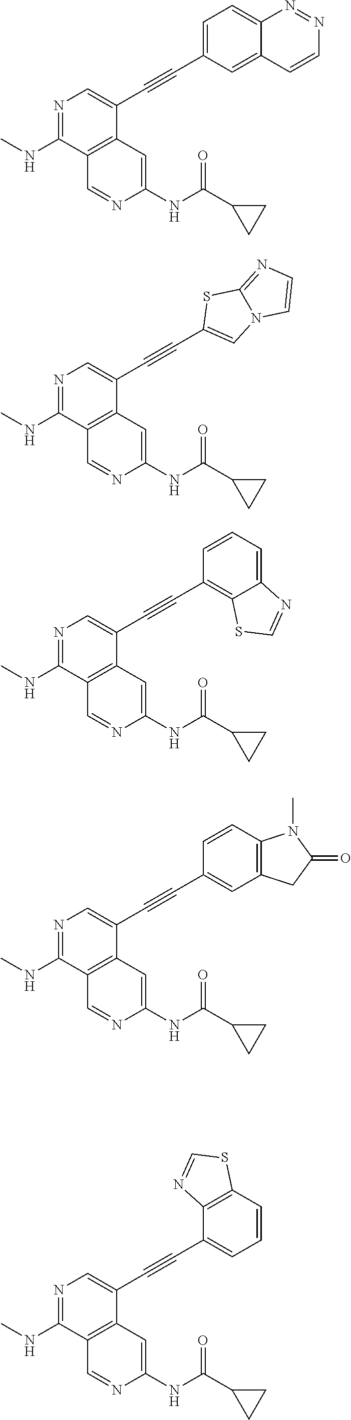

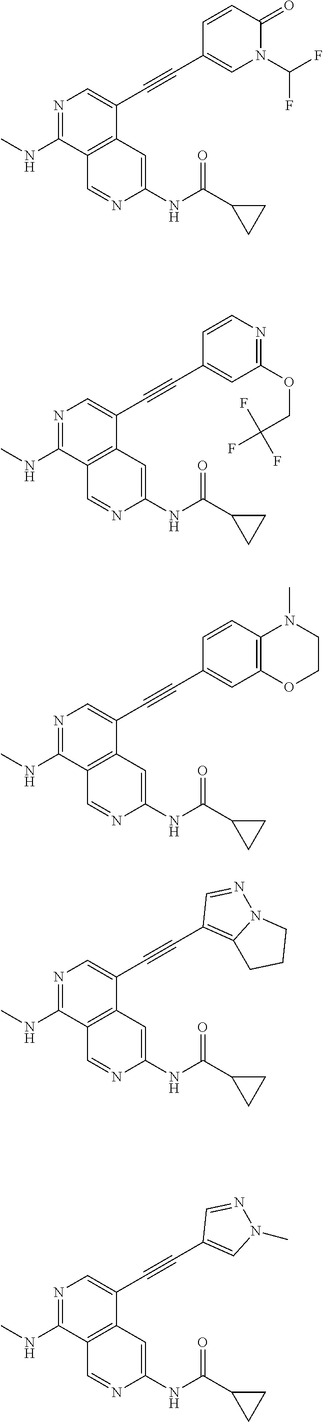

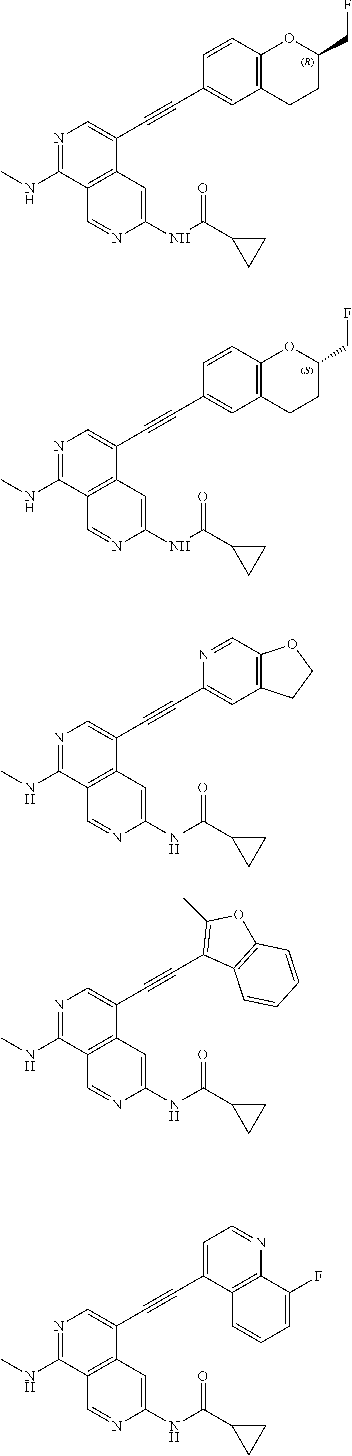

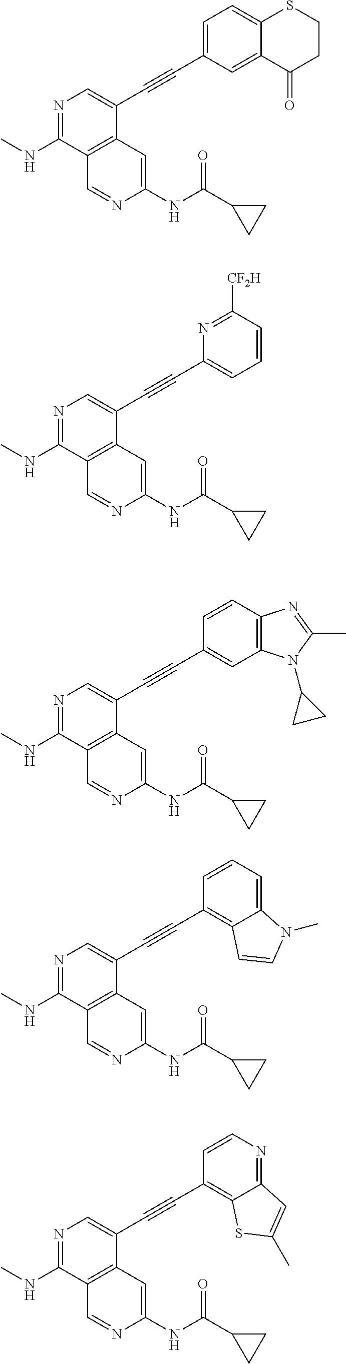

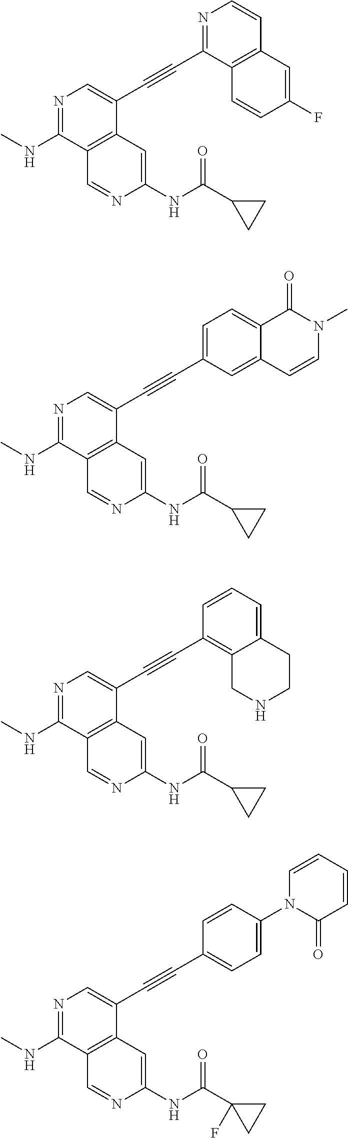

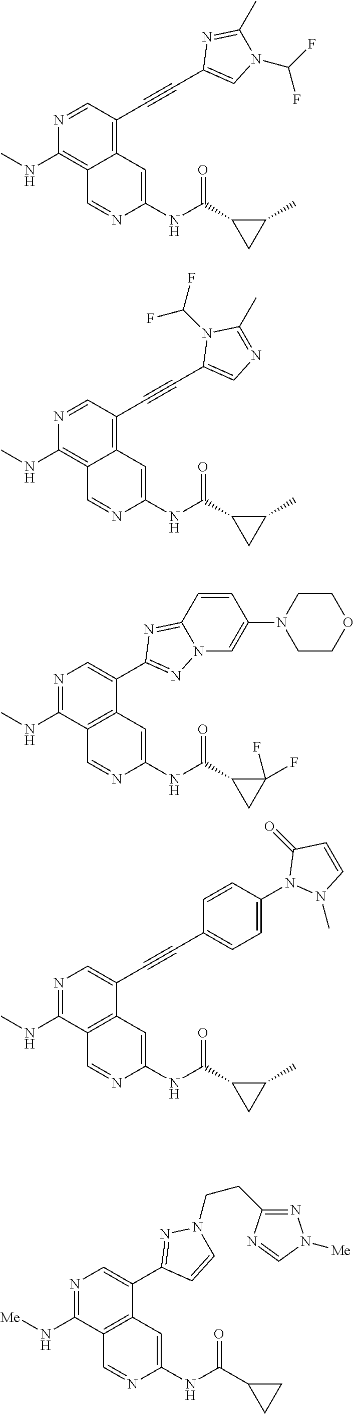

19. A method of treatment of a patient suffering from a neurological condition, wherein the method comprises administering to a patient a therapeutically effective amount of compound of formula (I): 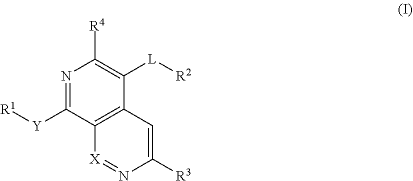
and pharmaceutically acceptable salts thereof,
wherein:
X is CH or N;
Y is CH₂, S, or NH;
L is a single bond, double bond, triple bond substituted or unsubstituted alkyl, heteroalkyl, alkoxy, heteroalkoxy, cycloalkyl, heterocycloalkyl, haloalkyl, halocycloalkyl, aryl, heteroaryl, —C(O)NH—, —NHC(O)—, O, NH, or S;
R¹ is alkyl, cycloalkyl, haloalkyl, halocycloalkyl, aryl, heteroaryl, methyl, CD₃, or H;
R² is H, halo, alkyl, branched alkyl, alkenyl, alkynyl, haloalkyl, alkoxy, cycloalkoxy, haloalkoxy, nitro, cyano, or a 5 or 6 membered substituted or unsubstituted aryl, or monocyclic or bicyclic heteroaryl ring optionally containing one or more heteroatoms independently selected from O, S, and N,
wherein the substitutions on the said 5 or 6 membered aryl or heteroaryl rings are: H, halo, alkyl, branched alkyl, alkenyl, alkynyl, haloalkyl, alkoxy, cycloalkoxy, haloalkoxy, thioalkyl, nitro, cyano, —CH₂-cycloalkyl, —CF₂-cycloalky, —CH(CH₃)-cycloalkyl, —CH₂-aryl, —CF₃, —CF₂-aryl, —CH(—CH₃)-aryl, C(═O)-alkyl, —C(═O)cycloalkyl, —C(═O)—NH-alkyl, C(═O)NH₂, hydroxy, —COOH (and ester thereof), sulfonyl, alkylsulfonyl, aryl sulfonyl, sulfonamide, amino, 3-6 membered cycloalkyl or heterocycloalkyl, 3-6 membered aryl or heteroaryl, any of which may have one or more substituents;
R³ is H, halo, alkyl, branched alkyl, alkenyl, alkynyl, cycloalkyl, haloalkyl, alkoxy, cycloalkoxy, haloalkoxy, nitro, cyano, aminoalkyl, aminocycloalkyl, aminoheterocycloalkyl, —NH-heteroaryl, —NH-phenyl, —NH₂, —NH—CH—CF₃, substituted or unsubstituted C(═O)cycloalkyl, substituted or unsubstituted —NH—C(═O)cycloalkyl, —NH—C(═O)alkyl, substituted or unsubstituted —NH—C(═O)cycloalkyl, substituted or unsubstituted aminoalkylaryl;
R⁴ is selected from a group consisting of: H, halo, alkyl, branched alkyl, alkenyl, alkynyl, cycloalkyl, haloalkyl, alkoxy, cycloalkoxy, haloalkoxy, nitro, cyano, and alkylhydroxyl.

20. The compound of claim 1, wherein Y is NH.

21. The method of claim 19, wherein Y is NH.

22. The method of claim 19, wherein R₁ is methyl.

23. The method of claim 19, wherein L is a single bond.

24. The method of claim 19, wherein X is CH.

25. The method of claim 19, wherein X is N.

26. The method of claim 19, wherein R³ is: 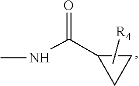
wherein R₄ is H, halo, alkyl, branched alkyl, alkenyl, alkynyl, cycloalkyl, spirocycloalkyl, haloalkyl, alkoxy, cycloalkoxy, haloalkoxy, nitro, or cyano.

27. The method of claim 19, wherein R³ is:

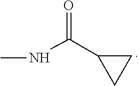

28. The method of claim 19, wherein R² is: 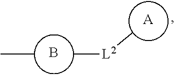
wherein L² is substituted or unsubstituted alkyl, heteroalkyl, alkoxy, heteroalkoxy, cycloalkyl, heterocycloalkyl, haloalkyl, halocycloalkyl, aryl, heteroaryl, or a bond linking the groups;
A or B are independently 5 or 6 membered substituted or unsubstituted aryl or heteroaryl ring optionally containing one or more heteroatoms independently selected from O, S, and N, wherein the substitutions on the said 5 or 6 membered aryl or heteroaryl ring are: H, halo, alkyl, branched alkyl, alkenyl, alkynyl, haloalkyl, alkoxy, cycloalkoxy, haloalkoxy, nitro, cyano, —CH₂-cycloalkyl, —CF₂-cycloalky, —CH(CH₃)-cycloalkyl, —CH₂-aryl, —CF₇-aryl, —CH(—CH₃)-aryl, C(═O)-alkyl, —C(═O)cycloalkyl, —C(═O)—NH-alkyl, —C(═O)NH₂, hydroxy, —COOH (and ester thereof), alkylsulfonyl, arylsulfonyl, sulfonamide, amino, 3-6 membered cycloalkyl or heterocycloalkyl, 3-6 membered aryl or heteroaryl, any of which may have one or more substituents.

29. The method of claim 19, wherein R² is phenyl.

30. The method of claim 19, wherein R² is: 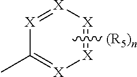
wherein each X is independently N or CH;
R⁵ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O) R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, and substituted or unsubstituted heteroaryl;
and n is 0, 1, 2, or 3.

31. The method of claim 19, wherein R² is: 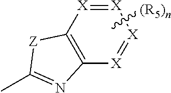
wherein Z is O or S, each X is independently N or CH;
R₅ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, substituted or unsubstituted heteroaryl;
and n is 0,1, 2 or 3.

32. The method of claim 19, wherein R² is: 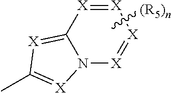
wherein each X is independently N or CH; R₅ is selected from a group consisting of: H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, substituted or unsubstituted heteroaryl;
and n is 0, 1, 2 or 3.

33. The method of claim 19, wherein L-R² is: 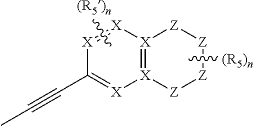
wherein each X is independently N or CH;
Z is independently 0 or NR⁶;
R⁵ and R^{5′} is independently H, halogen, hydroxyl, —CN, alkyl, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, or substituted or unsubstituted heteroaryl;
and n is 0,1, 2 or 3.

34. The method of claim 19, wherein L-R² is: 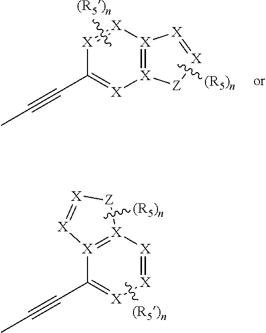
wherein Z is —N or O,
each X is independently N or CH;
R₅ and R₅′ is independently halogen, hydroxyl, —CN, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, or substituted or unsubstituted heteroaryl; and
n is 0, 1, 2, or 3.

35. The method of claim 19, wherein L-R² is: 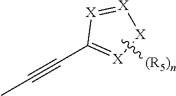
wherein R₅ is selected from a group consisting of: H, halogen, hydroxyl, —CN, haloalkyl, cycloalkyl, cycloalkenyl, substituted or unsubstituted cycloheteroalkyl, substituted or unsubstituted bridgedbicycloheteroalkyl, substituted or unsubstituted fusedbicycloheteroalkyl, substituted or unsubstituted spirobicycloheteroalkyl, substituted or unsubstituted aryl, substituted or unsubstituted heteroaryl, —OR⁶, —SR⁶, —NHR⁶, —NH(CO)R⁶, —C(O)R⁶, —C(O)NH R⁶, —S(O)R⁶, —S(O)NHR⁶, —S(O)(NH)R⁶, —S(O)(NMe)R⁶, —(CH₂)ₙS(O)R⁶, —(CH₂)ₙOR⁶, —P(O)R⁶R^{6′} where R⁶ and R^{6′} is independently alkyl, branched alkyl, haloalkyl, substituted or unsubstituted, cycloalkyl, substituted or unsubstituted cycloheteroalkyl, substituted or unsubstituted fusedbicycloheteroalkyl, substituted or unsubstituted bridgedbicycloheteroalkyl, substituted or unsubstituted spirobicycloheteroalkyl, substituted or unsubstituted aryl, and substituted or unsubstituted heteroaryl; and
n is 0, 1, 2, or 3.

36. The method of claim 19, wherein the compound is any one of the compounds recited in any of claim 17 or 18.

37. A pharmaceutical composition comprising a therapeutically effective of compound of claim 1.

38. The pharmaceutical composition of claim 37, wherein the composition further comprises at least one additional excipient.
